SEQUENCE LISTING (39 sequences)

SEQ ID NO: 1 - DNA, 234 bp, Mus musculus
tttttttttttttttttgaagttagaaaagtcttttaagtttttaaattaaaattgcaccctggtgcgatgactctaatttgggaactcaatccaaaggcagactctcctgagtgctggtcctctggcctggacacacagagactgggagcctgtcggacagttagaacagggaaggggcagaagcacccacatcagagggcagccaagtcttgacatctacctggtgacaagg

SEQ ID NO: 2 - DNA, 356 bp, Mus musculus
  misc_feature (1)..(356): At position 354 ′n′ equals c, t, g or a
ttgaaccgcgggcggcctgggctccttctgccatggcccccgcgctcaggtccttgctgtcgccacggactttactgctgctgctgctgagcctggcgctgctgggcgccgccgagcccgccaccgggagcgctgtccccgctcagagccgcccgtgcgtggattgccacgagtttgaattcatgcagcgcgcccttcaggacctacggaaaaccgcctacagcctggacgcacggacggagaccctcctgctgcaggctgagcgccgggctctgtgtgcctgctggccagctggacgctgaagatctttggccagtgatattccttgtcaataaatgcctgctagcatgccgnga

SEQ ID NO: 3 - DNA, 296 bp, Mus musculus
tttttttttttttttggttttagcagtatgtttttatttagggaacacagtatttgcaatagttctgacagccgtaaggccctccattttactagctgtttgctttgcaataaaatagccagaaattctgatatcaacattaataggatatttcttcatgcaggttgtaggagtatatacattgtataacactgaagaaattaatacatatgtacattatttacatagtaactatttattgtaaatagaaatgttttctaagataaaaagaaatcacaacatggtttctggtctgt

SEQ ID NO: 4 - DNA, 434 bp, Mus musculus
gtctgtaaaaatctgtttaataaatatacatcttagaagtaccaaaataattaccaacaaaatacaacatatacaacatttacaagaaggcgacacagaccttagttgggggcgacttttaagcacatgccactgaacacctggctcttacatgggaggacacactgggctcacttactaggtctatggtggttcaatcaaaagcacaataaataaaacgtggtcctttcattaggttctggaaaatcacctcccccccccccaaaaaaaatcccacaaacatgaaccttaagagacattttctttgaatttcagtgatctgtttccccggatttcacaaagacaacagccgaatcaccccagtaaaatgcctgggtctaggcgctgtgtggtgtggtgctaagtataccctttctcattttttttctttttct

SEQ ID NO: 5 - DNA, 417 bp, Mus musculus
tttttttttttttttttcaggtctcattttcgtttatttgaaattcggtgctcgtgtaagttttttctcttcccctcaaattttatttcagtaaaaggagacttgggcgaggtggataccccacagccggattcttccccccctgccccccagggtggctaatgctatctggggaagtcgtcatagggaagagaactatgggtgggctcctgcctgaggcctccaatctcagcccagtggacatatcacaggcagcttaaaaaaaaaaccctaaaaaaaacaccccaaaacacacatttaaataggtattcaagacagctttaaaaaatgcacccactcacacccccctcccttttctttttggaaaaaaaaataggaaaaaaaaaaaaaccaaaccgaattctcgcttggcctcta

SEQ ID NO: 6 - DNA, 375 bp, Mus musculus
tttttttttttttttttaaattcaagtgcctccatcttgtttacctatcactttaaaaagtactttaagtataagcttcaagagttttgagttctagagttttgcctggctgttgagatttggtgcaaattctcatgtgtgtttatataggaaggtgagctgaccatccactgcagaggatggaggtaattaacatgatctgtgctctttacaaaagagaatccagtgacctggcctgtgtgtgtgtgtgcgcgcgcgtgcacacacgctcgctagcgctcacattatgcatgaaaacttgattttcctgaagcatccatttccaaattggatagggatgaaaaagtaaaatttccattggattcaattagaagc

SEQ ID NO: 7 - DNA, 320 bp, Mus musculus
tttttttaagacaaaaggtttttattcaaaaaatgccaagtataaaaaaaaaaaataagatctcttttattcccctgtacagtatactttactcagataaaaccagcaggctgcacgctgctcaaagcacagccccagagaggatccggaagcacaccacaccggaactgtcttatgtacagtcagcagaccctagacccactccagcaagcagccaagggggcccctggagaagggggctacagcagctgtcccaccggagccccaacaccccagcaagcgtcacagccctcccctaaaaggtccccaagtcaggagga

SEQ ID NO: 8 - DNA, 255 bp, Mus musculus
tttttttttttttttctgacacagtagtaatccttccagactccttacacaatattacaactcccccagtacataaatgttcctataccatgcacacagcaacatggggtccactgatgtcgcaggcgacttttctaatggtggaacatagcacctcaagttctgccatctacacagtgaagggacgtgatggtcggggctccagagtgacagcaaactgcctcttgggtgacacgcttgtggcgaagtgcctcc

SEQ ID NO: 9 - DNA, 254 bp, Mus musculus
tttttttttttttttttcatccacaaaaaaaaaccaattttattaatccaagaaatgaaaaccatgtccctatggcttctagccccctcaaaaggatgaggctgggttcatgaacctggggtagaatgtcccctatccctcaccctcagcttattagactggaaaaagagtttgtgcaaataggacggtcaccagaggtaccaagtatgggggtgtcaggccagggcagcagatgaaggaggtgagggatctgt

SEQ ID NO: 10 - DNA, 728 bp, Mus musculus
ctgacaaggagatctgtggagtttttctccaagtgaaccaatcccctgtgtcctggctcacactgtggttagggtgggcacatccactctgccatctttaacacacagaaaccaaacatcagtgctccagtagcttactaatggaaagaggttggggtgagactcacccacagatgagccactctgctaggagccagctgtccctgtgctccttggtggggtctaggaggagtcaccctgttactagaattaccagcttgattcggcttcctctctgcatttccctggttctgccccacctgcgaggtccagagccatctcattcagggttcgccgcttcacagtcatccgaagggaggaggggatggccgccacccattcctttgtattattgcaactggtgctataaacaccaagtgctattgctcacagagacccatgaaggtaggtggaggagagacaaaaaacctagctggaactgaaagcagacatggagaccaggacccaggagaagagtcagtcgtgtatttaagagttcactttgtgaagcagtagctttcagtgcggtgttaaattataacgaaggcatggagtaccagggtgctttatatttagattatattttaagtcatggtgcatacttgagggggtttattaaaaatgaaagtactaacttaatgtctactaagtttaataaacaaagaatttggaagtaaaaaaaaaaagct

SEQ ID NO: 11 - DNA, 468 bp, Mus musculus
tttttttttttttttatgaagaagtattttaatagctgaaactcaaagtcactgtactccgggttccaggattcctaagaggcatttttggccatctcagaagccagggtcacccacctgtggtctcggggcatcaaattcctctgagtgccgactcggaggaaaagtgtaaacacgcccaagaccatggctgcgaggaccttcctcttgcctacgcatccgtcaactgcattagtcagacaacttaggctaagggaagggacaaatgttgactgtgtaagcaggaacagcccaagcttgtaaggaaaagcacgctgcatacatagcatacagaaggcaggacttctgcctgtgcacctgcaacattacatttgttttgcccgcaaactatcaagaaggattctgtcgggaggggtgttccctgaacaagcaaaatgttggacaaaggtcccgggtgtcctgcgtcac

SEQ ID NO: 12 - DNA, 473 bp, Mus musculus
tttttttttttttttttgctgataagaattctttttatgttattcgaataaaaaatacattcatacagaaatataacaatctcgcaaaaaacaatttcaaataaaatcttgtaaaacaaaattttacaaaaatcttacaaagattctttagataacagggtgcttcaaaaaaaaagaaataaagaaatttcactaatagaaatttttttttttaatttcaagcaaaagttcctgcttgattgaggctcagttgtcacctgaccagaatggactgcttagtattaaagttacagcatcgacacggacggcacccagccccagccagtccagcaacgtcgctgtgtttcataagtgagacgcgccagcacaagtttcctctctcttctgtttaccttcttacttaatggaattgctatggataagcacacagcagggccaaaaaaggagttttccaaaatccagcaaatcaagtg

SEQ ID NO: 13 - DNA, 316 bp, Mus musculus
cataaaacagctgaatacatacaaaactgtggttattgatgagctgttgcttttgttgcctttcttgctgtttattaactgacaaagaccatgctaacaatcatctttatgaatgggtgctagctgatgggcagatcatttgtatgggctagataaaaatgttaataatgaagccatgtgctcaggtttgaattgaaaacaattaaaaaaaatcacttgctattttgttttcctttgaagaatataaagttatttcagttagatcatcagtaaagttttatctgggctaagaaaatttcccttgagttcttgtgct

SEQ ID NO: 14 - DNA, 598 bp, Mus musculus
ttttttttttttttttttttttttttttttttttttttttttttaagggcaggggtaatctttttttgacccggaagaggaaaaaattcatgaaaaattgccgtgggtttttcaaaaaaaaatttaaagaactctttgtttcttgatgaggtatcctaggtaccatggggatagactggaattggccttctcagggagacaggatggaatgcaaaccccaaaatttaaactggggatgctgtttggataggaagggatgatgggggacccctggggagaagcctccactcaacactggaggctcaggccattcttcattctcggccctggaggcaacgatgggcattggataggccaatgatattttctttaaggattccatgctcctcacatagttgggcaaacctttccttgatgtctgacctcaaatctggttctcggccatagagccccatcagcttgaaggttcccccatcctttccgtaatgagatgagccataagaaagttatcatagtctgcctaagtataggaaatggattgaatcatcatcccgcacagatattcaccagcttttttgtttgcagcaccctagataat

SEQ ID NO: 15 - DNA, 580 bp, Mus musculus
  misc_feature (1)..(580): At positions 138, 151, 156, 173, 356, 358 and 361 ′n′ equals c, t, g or a
tttttgtctttcccatgcttttattggttgatagagaacaaaagaatatcaaacaaatgtgtttacatatcacaggggacttgatctgtgcataaaaacaaaataacaacaaaagcccttacatatagaacatacatncaaaaaagatttnttttngaaccaacagcatgaanacttcttgagattcattgcatgccactagtttctatgcactagtgaataaaactgatatgataaaagacccacatcatagcagagattctgaatacaaagactgctaatggttaactgttgctgttaattgcagggggcaagaaaatgtccctttgctccactacaatggcctgaaacattgntnggntttctttctttctttctttttttattacgtattttcttcaattacatttccaatgcgtatcccccaaagcccatacctccccccccactctcctacccacccattcccattttttggcccctgcgttcccctgactggggcatataaaagttgcctgaccaatggccttctcttccagtgatggccgactaggtcatcttttgatacatatgccgctag

SEQ ID NO: 16 - DNA, 542 bp, Mus musculus
ggtaactacagcttagaaacccaatatcagttgtacttaacatctttaagagcatgatcataaaggctattttgacagttcattttaacatttagagttgataagggtttatatctcatctgtccatattgtttgcaaaggaacaaggttttatgtaggtgagagaaagcatttgtacgaagttagatttgaacatagataagataggttattccagttgagaatattgttcgaagaatgtctgtgaatgcagacatgggtcctgcttagtgccatgggtagactgagtcttctgtaagtcatcataacatagtaattttggtcctgaattttacattaaattatttgaatatatacagacctaaattttaaaatctgtacatatataattttgatgtattaagatggataatattgctgatttaaatttaattatgcacatacttaaaaggaccgaaatgtctggtaaagtaaattgtaaataaggatatgtagcctttaaccttttaaataattagattttttaatgtgggtctcctcag

SEQ ID NO: 17 - DNA, 476 bp, Mus musculus
gcttaacgtgtttgatacctaacttcatgatcacgatgacctgtccttaccaagagcaaagcaagacatagctgtggctttgcttctgtaaccagctcatggggtgtgaatgtcatgaagttgtctcgctgatatatgtttaatgtcatgttatatgagatgatcatgtgatgtgtacagactatgttgagaagtgccagtggtagtaactgtaaagtctgactcacaccattaatccctctaaagtacacagccttctgcctctgtgtggagttgtcagtacagctcgtcaaagaaaactgcctaatataaaaatcatatatatggtgataattttccctcttttgtagtctgcacaagatccataaaagattgtatttttattactatttaacgagtgattaaatgtagtctgcacagtgagcaagagttcacaattctttttatactgctggattttgttgtgcatcatttaa

SEQ ID NO: 18 - DNA, 448 bp, Mus musculus
ggaacttcaatagaatatcgtcccttgtctgaggaaagaccacctcaaccacatgcctatctagagattgaataaaactgtgaggagacactgtgtggatgccttaagatggctggcgctggctgcagggaacaatctatttcagctcatcttcataaagcccaagatgctcccgtgcataaccttgttgaagaaatgagaaatgcggtgctggctactttgatgtgtctggggaggctggcgtcattggttaccatcattaagtcactgtccagtgcccaagccttccttcactaccactcatatgtctacatccattcattcatctatgcaagcttgtgtttttgtggtttgtattttacacctggacatcaggtttaacattggtgttttaaataatttttaaaccaaagtattttatgagtaagtctatgtgctctgtttttgt

SEQ ID NO: 19 - DNA, 385 bp, Mus musculus
  misc_feature (1)..(385): At position 21 ′n′ equals c, t, g or a
cttcggcacaggcgcacggtnaggagtttgcctctgcctcccgagtgctgggatgaaaggtgtgcaccaccacgcccgactccaatttttttctttattattttttttgtgggtgtgaattgaaagtctaatacgtgtttctctgtttatggacccggatcccacgccagcgtccactggtcactgccgctgcccacagcgacgtcgcccacgagagcacacacgtagacccagacgccgtggtcaagaatctctgccatccccacgggacggacggacggacggactccacaaggtgcgcgtgtcaccgaggccgccagaatggagccactctcacgattctcaacagggcctgaccgcgtacagaaatgtccccctcaataaa

SEQ ID NO: 20 - DNA, 392 bp, Mus musculus
tttttttttttttttttcacattctacttctgctttattgcacaatatctataagaaaatattaattttaaattagaaaacataaaaatgctttcaatatagaagagaccatctgattttaaagacaatactctttaaggatgtctacccaggaaataacaaactttcacaggataccaaactggacagacgcttcagcgaaatcggtacaaagctgttttaaaaccagtctgagacacttaatgcagcctctattatcactcttcattaatagtctagataccactcaaagtgttccttacatttcccaatgttgaggcatcttcagtgtgatcttcagtgaacactatgaacatacagcatgccttccatgtcagaagcagcactcggga

SEQ ID NO: 21 - DNA, 426 bp, Mus musculus
tttttttttttttttttcaacacaaagctttcggcatattttattgtgaaatattggcttagaaaaaagtacaaaatgccaacagtcagatgtatacaggtacagtattttgttaatctatacatgtgaccagcttaacaggctcatcggaaaatgcatatttagaccaatatactctaaaaatgactgtaatattcacaggctattaatacatgataaactggagtctatatgaccctcactgggatatgctaactgcagcaaacaggctaaaagcacaatacagcacaccatagtacctctggacaggtccacgtttaaagagtatcaattcattagatcaatacactacagatctgggactgagactcaatcactaatttatacaattgcaaaaaaaatttaggttataaaataaagttatgg

SEQ ID NO: 22 - DNA, 339 bp, Mus musculus
ggcatcgttggtttcttggctttgtgcatgttaaacctggtatttctaatgatacagtttttcatatgtacataaatttttgtatgtaggtgtgtcggggagggcagaacaacttgcaagtttgatctcaatgtgggggtcttaaactacctggtcttaaagctttctcttcttcctagggtgacacgagtgaaccctaccctaaaatggtttaaagataacatgaagggtccttaaaataaattgaagaaatcctcaaatcttctggtgacctggatggacagcaaaggaatcaaatccctcaaataatttatttgacttaaattaaaaataaataaa

SEQ ID NO: 23 - DNA, 385 bp, Mus musculus
tttttttttttttttttcacttaatcagttttattgaaattcatcagatgaaaagaatcagtgttccatagaaaaggattaatgttgtactttgtaattttaaacaccaacaactgcttaacaaaaattcatgtgagttttacattttgttgttctttttacttaaggagtgaagtctgatttcaccaaaactccctgtactcagtctttcccagtaaaagtaatctttgtttcaaaattccctattcacatatacttagggtcccaatgaagtgtccacatttagcagtacaattaacaatttacagtgtttttgaaagtggaactctttctaaagaacaaaatttctaaagggcaaatcagctcttaaatgtgaaatttacaa

SEQ ID NO: 24 - DNA, 217 bp, Mus musculus
tttttttttttttttttgacttttaaaggatttttatactatattaaaaaaaacacaaaataaaaaagggatccatcaacatatattttagaagtccatccaagagtttcagtgtccaccagccatggacacaacagccatgcctgtgtcattgttcagtctgtagctcatgtaaggatcaaggaggtgactttaagttacaatcaaacttgctctg

SEQ ID NO: 25 - DNA, 370 bp, Mus musculus
tttttttttttttttgtgataatacaattttctttagtaaaaataatttacagcatcaataacatatacacaatttcataactgaactttacctccaatatatttctatacaatacttaacattattgaacttaaaactgttatgctattttgttggctttaaataacaatgatttgtagttacttaagagatatataaggttttacagtcacatattatgcataaaattgataaaacaaacaaatcagcacagtcaccttgaaagggaaaataacccactggaaaaaagccaaagttcatttccttccccgttttttactccccaaatcaaatacagtatcattgtccagtaccttagcatcttctctc

SEQ ID NO: 26 - DNA, 496 bp, Mus musculus
  misc_feature (1)..(496): At position 10, 63, 371, 372, 373, 374, 492 ′n′ equals c, t, g or a
gggaggaaanggtttatttggcttacactttcatactgcttactgttcatcaccaaggaagtngggactggaactcaagcaggtcaagaagcagaactgatgcagaggccacagaaggatgtttctttattagcttgcttcacctggcttgctcagactgctgtcttagagaacccaagactaccagtgcagagagatggtcccacccacaaggggcctttccccttgatcactaattgagaaaatgtcttacatctggatctcatggaggcatttcccaactgaagctcctttctctgtgataacttcagcttgtgtcaagttgacacaaaattagccagtataattgacaccttgtcaatttgaggcgnnnncacatcactagtaatgctcaactcttagtttcttattcatgcccaagatatacacaactttgaaagccccacagactttacatattaaaagttcaatgcttttaaaatatccaatatncgtg

SEQ ID NO: 27 - DNA, 443 bp, Mus musculus
ttcacatagcatcaagtgactacagatacaacactagaattttgatgtgtattatttgaacacgtttatatatgagaaaaatttgaaatttaagttacaaagtgagtattttttagcacatttagtagtgactatttatataaactatcattttgtttgggtgaccaaatgaaatttgaacataattaaagaacatataactttcaaacaacattaatacagttacagacctgttacactgagaattatgtacacgtattaaagaggactgaggtttcttaaacccattcttttgaaaggtttttaatccttcttaatgtaattcagtgtaaaacttcaaaaagtctactcggtgtcacagcaggatgaggtggaataatccctgctgatcactgagcaaactgtgtttgtcatctgattctcctgacaaccactatacttga

SEQ ID NO: 28 - DNA, 398 bp, Mus musculus
gttcatctcaaagccctttacactttggagccaggcctgttcaattttacaaataacacgttttgcttgtagtcagggtgctgtgacaacatgaagagttcgagctacctcccaggatgagtgcaggttttcctggtgacaaatctcaggcattcaaaggttctgttagcagtgccatacttgtgggtttacagagatggtgtgtctggctgagaacaaaggagccgtaccagggaagttgaagcctatcttcattttcaagtgcaggctaaaataaggtctgaggaaaagaatgagcagcgtgtgaaggataggaacagaaatcatggaagatctgtttgtccaggccagtgtttcaccataagagatcactttaaaataaatgagatgaaataatt

SEQ ID NO: 29 - DNA, 430 bp, Mus musculus
tttttttttttttttttcactttgcaaactgttgtttttatttggattaaaaagctttgatgaacaaacatatgatacatacatggtatagaaccagttaccacttaaaacctcagatgatatagtacagaaattagaataaaccaaattttaatcaggtctggaaacaaacgttcaagctgaaagctgtaacatccatttggagattttacacacctgtcccttacccactcagttcttccttagaaggggaaggcagacttagtaaaaccaatgttttttaagtcagctgactgacattctattttaaacctataggtccccaacccaatataaacaaaattcttcacaggaacttacattttgaaaacaattaggccattatatttcctaagccacattactacagttcttaaaagtcatgagaaag

SEQ ID NO: 30 - DNA, 520 bp, Mus musculus
tttttttttttttttttattcatgcttgcctagggatggggaatagatcattcaataaaaacatacagtaaaaacagggggtggggagggggaaggcttatcatgtacaatgtttaaactacaatagtgatgtaccttaattacttccatgcacacaagtctaacattacaagtttttaaaaaataaacaccattaagacttctaggagcattttataataaattcctaattttttctttgtagatagatcaagcacctccaaaatacagattcctatacacagtgagcactttacttaacgtacatggacagcctcaggacgagctgacgtctcggatcagctcggcaggcaacaaaccatagtgccaaatggaaagaagggcagttgcaaataaacttaaaactaagttaacttttataattaaatacagaaaatatactgatttgctaaaaataaataagatgtgatgtatttaacacttcactataaagaatgaacaccatgacatcctcgtgccg

SEQ ID NO: 31 - DNA, 390 bp, Mus musculus
ttcgtcgacatgcaatagcagggggaaaggggacaggtaacaggagtacctttgttttaacagacaaaagtaatctccaggccttagggactcataaagatatgagtacattgcttattggaaacttgtaggttcacatattattttgactaaggtatatggatttccccccacttacataagtaaacccaacataaggaggaccattccttttctttcattcagatgacctttttctgaatctgggcactgaagggatgcataaaataatgttaatgttttcagtaatgtcttcaagtagaagactttctgttgaactacttataagaaagtaaaataaatattgtctttttgtatgtcaaaaaaaaaaaaaaaaaaaaaaaaaaaagg

SEQ ID NO: 32 - DNA, 238 bp, Mus musculus
aattcgtcgacatggcacagcttcctccctaggcgtgagactccggctccttcactatgagacttctagccctttccggtctgctctgcatgctgctcctctgtttctgcattttctcctcagaagggagaagacatcctgccaagtccttgaaactcaggcgctgctgtcacctatctcctagatccaagctgacaacctggaaaggaaaccacacaaggccctgcagactctgcag

SEQ ID NO: 33 - DNA, 383 bp, Mus musculus
tttttttttttttttttgattaatcatgtatttattttagttcagttaaaacaaacatacattgtttcattgaaaccggtgtagcatttactctctgccaacaggccacagaggcatgttggcctctaacagtacagctgggacatttacactatatacatacaaggagtcctctttcattgagatcacacttgacatgttaccaaatagccatgtggtagctactgcattttgaaaatttagacctcatttaaagctttagattatctgaacttaagttgatgtccaaccagtttattgcaaatacaatttaaacaagttattttcaattttagtattatacacaatgtcaatttcttaagattgaattctctgtacaagta

SEQ ID NO: 34 - DNA, 249 bp, Mus musculus
tttttttttttttttttgaccacacagacatggagttaccaattgtagaaatcgaaaaaattggcatcaactctcgaagtcagagtgaactcttgcctgcgggttggcttgactacgcccaggcactgagctgcctcaaccagctagggagctatgatgatgctgagtcctgttttcatgatgtcaccagatgtatgtagtaggtaatttgtctcacaaaggcctttgtaccaatggctgtgaagcttg

SEQ ID NO: 35 - DNA, 469 bp, Mus musculus
tttttttttttttttttaattacgcaaaaatttatttgaccaaaatgtagaaaaagtgatactattacgtatgatacaatagcaagaacataaagaacagagtagattttatcaattgcagtttgctcaaagttcatctccctggtagtgtgatgcttaataaataggagtgaggggcagggttcagataagctagaagcagggtgactttcagtcaggactgtaaacttgagttggccccgcactgctggggaatgtggaatgtttcagtgtgagatgttaactgagaaaggaaaactacaaagccaaaaaatgtgcagcctacagagcactccacatccagttcaatcaggaggtcttggtcttttattagtgtcattcccgaaggaattcaagtcttggcaaagtcaagcttccatttgttgttccctgaacaatggagatgaagttaagccaatttctttttcta

SEQ ID NO: 36 - DNA, 355 bp, Mus musculus
tttttttttcctactagactaattaaaaaaaaaagtcaacattgatatcgattacttgcatgaagacacaggaaaacaggcgacatacactccttaaagacacacaagataagaagatgaaacttcaggtacataagaagtctgtacaaaaagctagatttgatactctcaaaaagtgaagggtcctacaacagcatagagaaataatttaatgccttccagaacagaactccagctctgtggaggttttcctattctatgggggcagatctcatgccaacccacagagcaggcgcttccacctcctatccgtttatgcggtagttttcatggatttccggccggcctcgtgccg

SEQ ID NO: 37 - DNA, 217 bp, Mus musculus
atgttgaaaaatcttatttattttcttaattaaaaagaagtatatttgtgttaaatttagaatatttagctttccattgatcttcccaatacttgtcagatagatactaatagtgctcagaactgtgaaagttaatatccaataccatattttctaagaagaccctcaaggcattggggattttaatttaaaaataaaagaattttaattagcattg

SEQ ID NO: 38 - DNA, 362 bp, Mus musculus
tttttttttttttttttgaaaaagaaattttttgcctttattagaatggcattaggccttaaatatgccaattttggtaatcacattattgttttaataagaaacgactctacagaattgcaatactggtccaacagtcttgtccttcttttaaagcaagaaacagaatgtaagtaaccagaaagcagggcaggcatcggctaacccaggagactagcttcttagatccaagcatttgcagagagaaccgttgggctggggaggggtggagcagctcgagataactggaacccagagtgtgcgccaagtcccatgaggctgcttgttgaactcatcttttccttggtcacattgtttccctc

SEQ ID NO: 39 - DNA, 280 bp, Mus musculus
tttttttttttttttttcaagagaaaataggatttatttatcacatttgagatgaacacacaggactgtcccagaaatcttttacatatatattattgatagattggatttattcagcatacagaaaaagcaaagcctgccataagacacttccctcttgagaacagcaagatgtcaggcagtgtctgtaagttttcaccacgggaattcacattgagcaaaaagaccgggctcacaggtccgttcccaggtcctgctgcttctatttcaaaggctttca

(19) United States
(12) Patent Application Publication
(10) Pub. No.: US 2004/0038292 A1
(43) Pub. Date: Feb. 26, 2004

(54) Wound healing biomarkers

(76) Inventors: Martyn Frank Burslem, Sandwich (GB); Claire Michelle Johnson, Sandwich (GB); Lisa Cooper, London (GB); Paul Martin, London (GB)

Correspondence Address: PFIZER INC., PATENT DEPARTMENT, MS8260-1611, EASTERN POINT ROAD, GROTON, CT, 06340, US

(21) Appl. No.: 10/175,184

(22) Filed: Jun. 18, 2002

Related U.S. Application Data
(60) Provisional application No. 60/305,346, filed on Jul. 13, 2001.

(30) Foreign Application Priority Data
Jun. 18, 2001 (GB) 0114869.1

(51) Int. Cl. G01N 33/53; C07H 21/04; C12P 21/02; C12N 5/06; C12N 9/64

(52) U.S. Cl. 435/7.1; 435/69.1; 435/226; 435/320.1; 435/325; 536/23.2

(57) ABSTRACT

This invention provides biomarkers such as genes and the corresponding mRNA transcripts or protein products that are identified as being involved in wound healing processes. Also provided are methods for identification of compounds useful for the treatment of wounds, wound healing disorders or inflammation and compounds identified by such methods. Methods are provided for monitoring the progress of wound healing and for identification of individuals with wound healing disorders.

### CROSS REFERENCE TO RELATED APPLICATION

This application claims the benefit of U.S. Provisional Application No. 60/305,346 filed Jul. 13, 2001.

This application claims the benefit of U.K. Application No. 0114869.1 filed Jun. 18, 2001.

This invention relates to the fields of molecular biology and wound healing. More particularly this invention relates to methods and probes for investigating and evaluating the presence of RNA species that are differentially expressed in wound and normal tissue. The invention also relates to use of specific genes and their translation products to monitor wound healing and/or to detect disorders or diseases characterised by impaired or excessive wound healing. The invention also relates to methods for the identification of compounds useful for the treatment of wounds, inflammation and wound healing disorders, compounds identified by such screening methods, the use of such compounds in the manufacture of medicaments or in methods of medical treatment.

In recent years much has been learned about the cell biology of wound repair, but the current understanding of the regulation and coordination of the various processes that underlie wound repair is still poor. Effective and wide-ranging therapeutic intervention will largely depend on the identification of the full repertoire of signals controlling each step in the tissue repair process and a detailed understanding of the mechanisms involved in effective tissue repair.

Most skin lesions heal rapidly and efficiently within a week or two. However, the result of this process is not perfect; often there remains a connective tissue scar where the collagen matrix has been poorly reconstituted, epidermal appendages that have been lost at the site of damage do not regenerate. In some conditions, wounds become chronic, i.e. do not heal at all.

Most wounds will cause leakage of blood from damaged blood vessels, the formation of a clot serves as a temporary shield and provides a provisional matrix over and through which cells can migrate during the repair process. The clot also serves as a reservoir of cytokines and growth factors, released as the activated platelets degranulate, which provide chemotactic signals for circulating inflammatory cells to migrate to the wound site, initiate the tissue movements of reepithelialisation and connective tissue contraction and stimulate the angiogenic response.

The inflammatory component of the wound healing response was long considered necessary, because it would help eliminate the damaged tissue and any contaminants that may have entered the wound environment and would allow new skin to form.

A genetically modified mouse model is available that lacks macrophages and B-cells (McKercher, S. R. et al (1996) EMBO J. 15, 5647-5658). The mouse model was generated by a knockout mutation in the gene for transcription factor PU.1, a member of the Ets family of transcription factors, the expression of which appears to be strictly limited to cells of the haematopoietic lineage (Lloberas, J. et al (1999) Immunol. Today 20, 184-189).

When the present inventors investigated the wound healing ability of the PU.1 knockout (KO) mouse, it was found that PU.1 mice healed wounds perfectly, with little or no scarring. This provided strong evidence that, contrary to established belief, the inflammatory response is not required for wound healing and indeed may be a factor in scarring and/or fibrosis.

The PU.1 knockout mouse was used to investigate genes involved in wound healing, and, when results from the knockout mouse are compared with the wildtype mouse, to dissect out the inflammatory components in wound healing. Analysis was carried out to identify genes that are upregulated or downregulated during a wound healing response, occurring with or without the inflammatory response usually occurring in healing wounds. One method for this type of analysis is microarray technology. With DNA microarray technology, it becomes possible to monitor large-scale gene expression over time. Prefabricated arrays of large numbers of especially designed oligonucleotide probes, e.g. as manufactured by Affymetrix (CA), enable simultaneous hybridisation-based analysis of the expression of thousands of genes. Using this method, four stages during repair of a neonatal skin wound in wild type and PU1 knockout mice were investigated: 30 minutes, 3 hours, 12 hours and 24 hours. This allowed the identification of the immediate early wound response genes, the early and late effector genes and genes required to complete reepithelialisation. It also allowed identification of genes involved in the inflammatory response.

The present invention is based on the analysis of genes, the expression of which is upregulated or downregulated during a wound healing response, occurring with or without the inflammatory response usually occurring in healing wounds.

The present invention provides novel targets (genes, or the corresponding RNA or protein products), for therapeutic intervention in diseases or disorders involving impaired wound healing, as well as such targets for therapeutic intervention in diseases or disorders characterised by an excessive wound healing response. The invention also provides the use of antagonists or inhibitors of these novel targets for the treatment of wounds or of disorders characterised by excessive wound healing; the invention also provides the use of agonists or activators of these targets for the treatment of wounds or disorders characterised by impaired wound healing. It will be appreciated that such antagonists or inhibitors can be compounds that reduce the activity of the target, antibodies specific for the target, as well as antisense oligonucleotides or ribozymes or transcription inhibitors, that have the effect of reducing the amount of the target produced. It will also be appreciated that agonists or activators of these targets can comprise compounds that increase the activity of the target itself, as well as compounds that increase the expression of the target. Increased activity of the target could also be achieved by delivering the target itself, either as protein or its coding region in an appropriate vector as gene therapy.

The present invention also provides markers which are useful in monitoring, for example, the state of healing of a wound; these markers can be used more generally for monitoring diseases or disorders characterised by impaired or by excessive wound healing. In addition, the invention provides markers for wound inflammation. These markers are useful in the clinical assessment of progress of healing, and can be used to aid in the selection of the appropriate therapeutic intervention.

Definitions

Coding sequence or coding region refers to a nucleic acid molecule having sequence information necessary to produce a gene product when the sequence is expressed.

Antisense oligonucleotide refers to a small nucleic acid molecule, typically between about 10 to 50 nucleotides long, which may be unmodified RNA or DNA or modified RNA or DNA; it is designed to hybridise to the respective transcript, and thereby reduce its translation into protein, e.g. by blocking translation or by facilitating rapid degradation of the respective transcript.

Ribozymes are RNA molecules capable of catalysing RNA cleavage in a sequence specific manner.

Antibody as used herein includes polyclonal and monoclonal antibodies, single chain, chimeric and humanised antibodies, as well as antibody fragments, whether produced by recombinant or proteolytic means. The term is also meant to include the products of any antibody-derived expression libraries, e.g. single-chain Fv or Fab fragment expression libraries.

Marker/biomarker refers to any molecule derived from the gene, e.g., a transcript of the gene, a sense (coding) or antisense (non-coding) probe sequence derived from the gene, or a full length or partial length translation product of the gene or an antibody thereto, which can be used to monitor a condition, disorder, disease, or the status in the progression of a process, e.g. a healing process or the progression in a disease. Biomarkers may be labelled to assist detection, the choice of label will be directed by the nature of the biomarker, suitable labels include radionuclides, enzymes, fluorescent, chemiluminescent, or chromogenic agents as well as substrates, cofactors, inhibitors, magnetic particles.

The gene sequences identified by accession numbers in Table 1 and provided in the sequence listing are mouse (Mus muscularis) sequences. Mammalian homologues of these mouse genes, particularly human or rat homologues are included in the scope of this invention and, given the mouse sequence information, the corresponding gene in human, rat or other mammals can be readily identified and isolated using techniques known in the art.

### EXAMPLES

The invention will now be illustrated by a number of examples. While they are typical of the methods that might be used, other procedures known to those skilled in the art may also be used. All Tables referred to are found at the end of this section. References are made to the following figures:

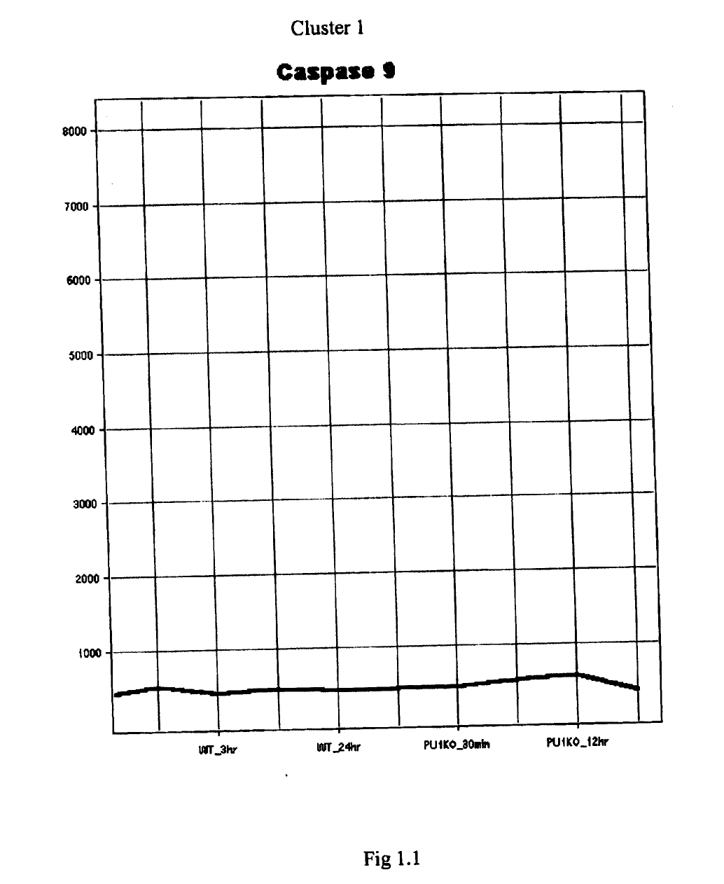

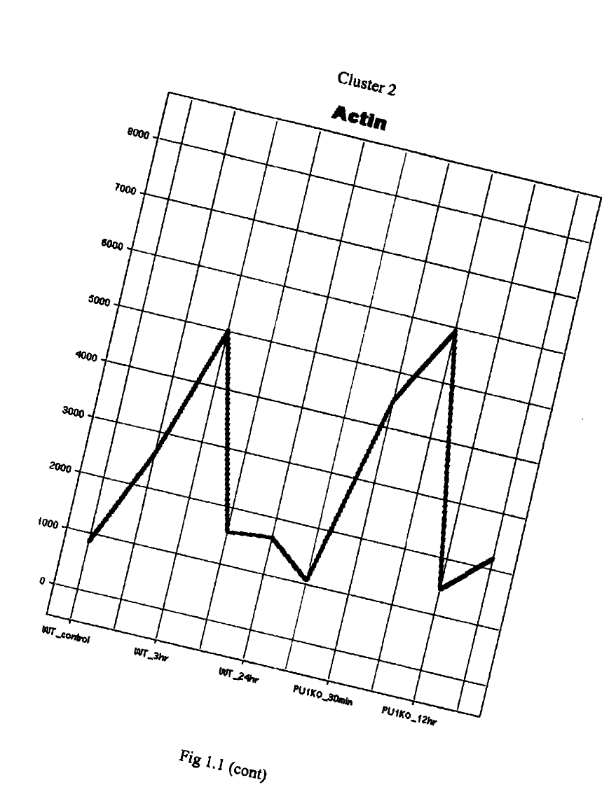

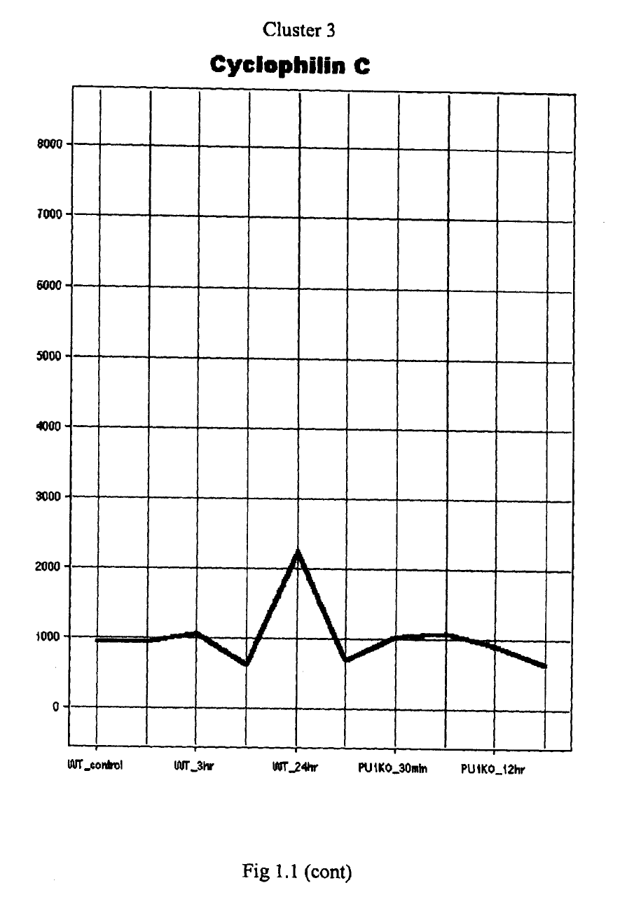

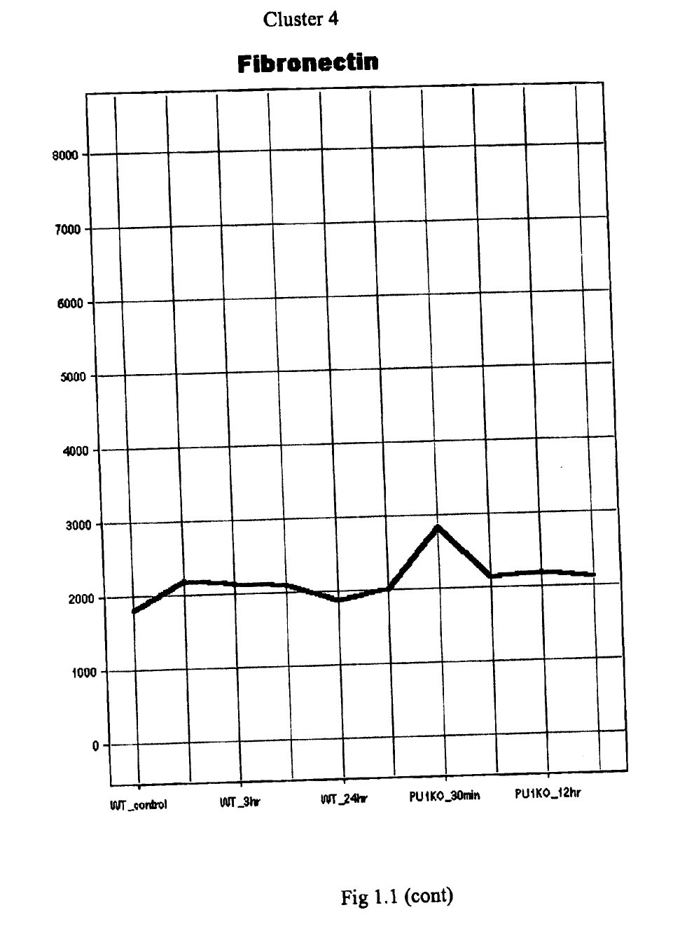

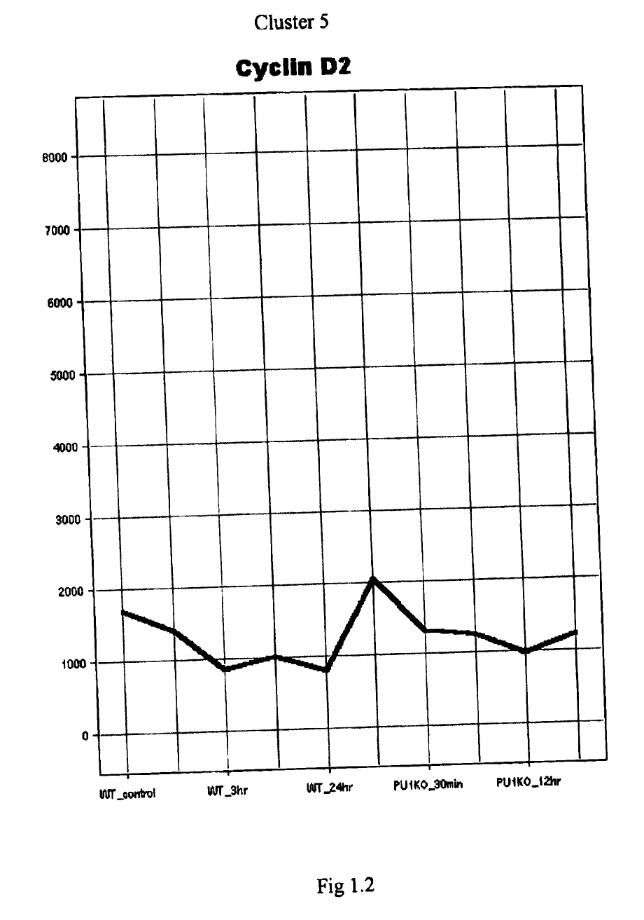

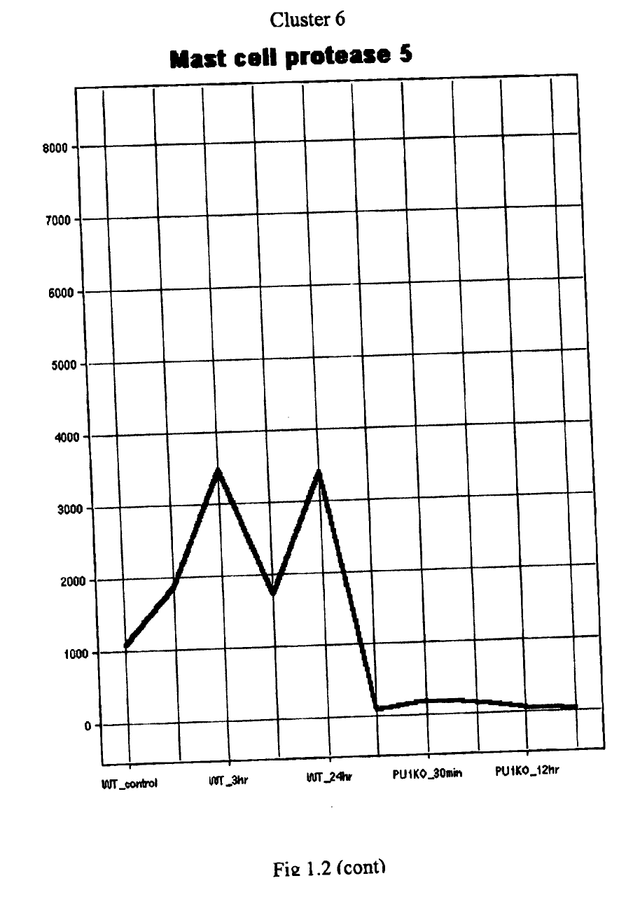

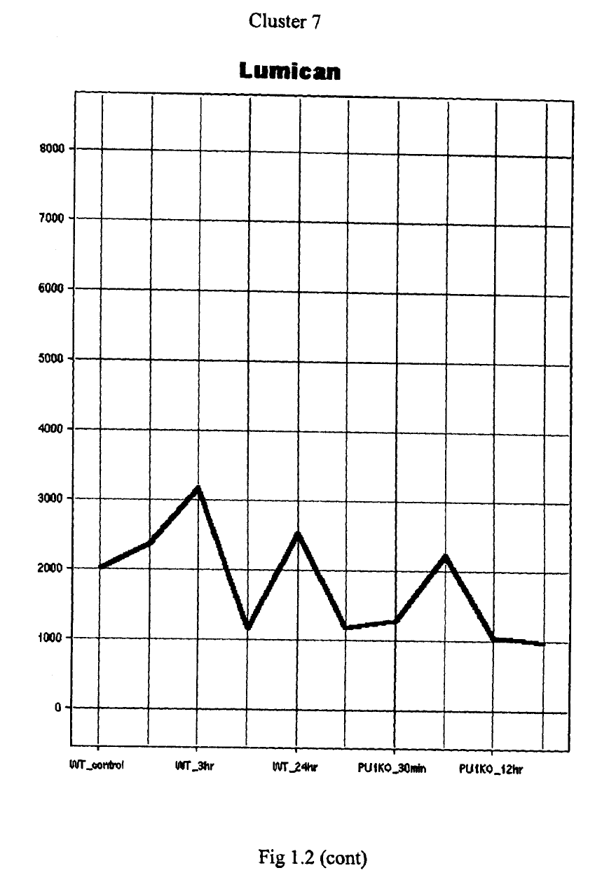

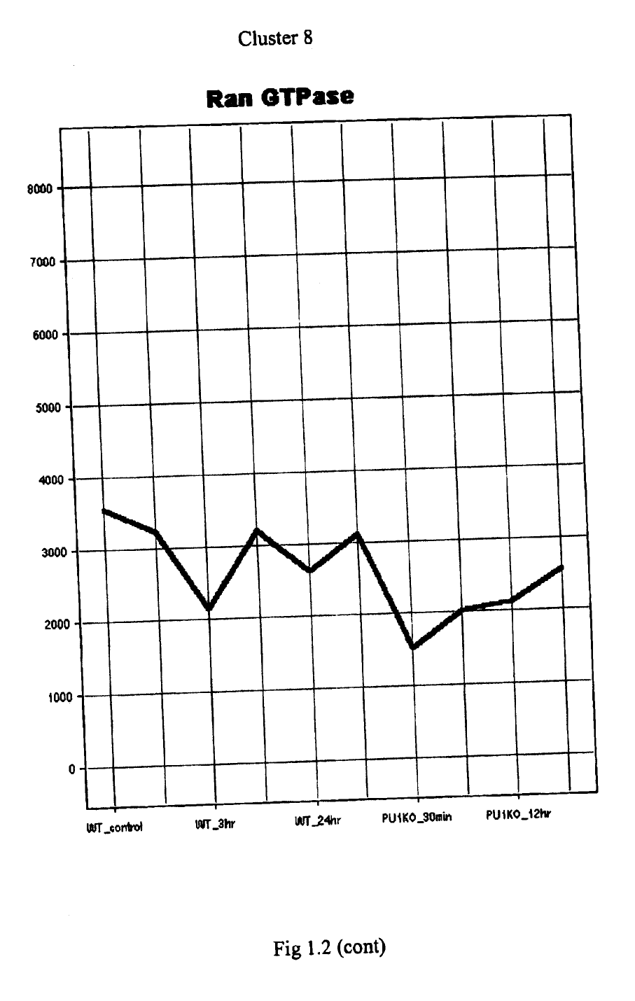

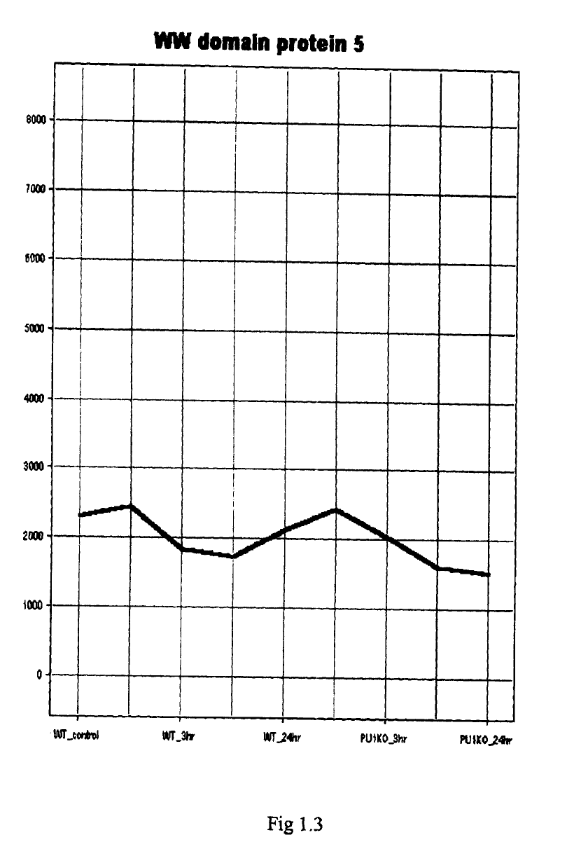

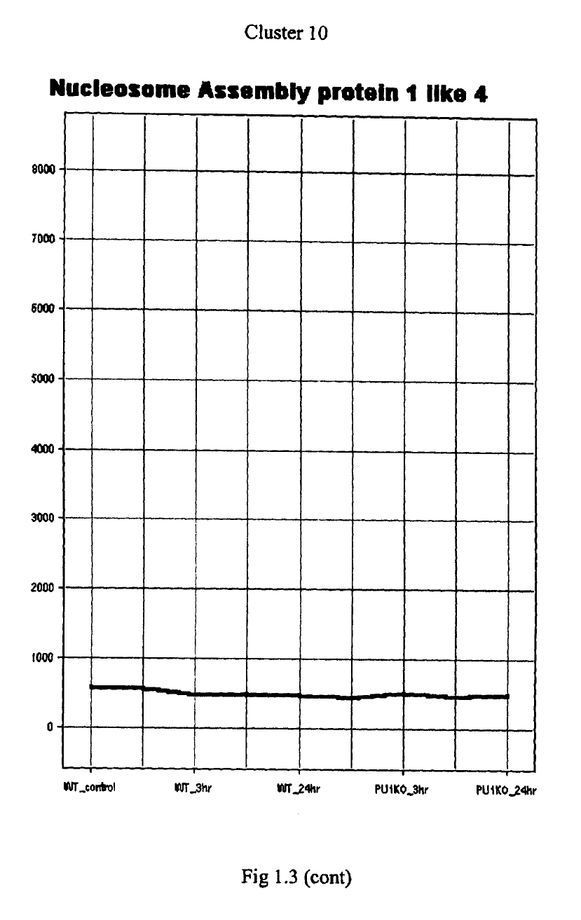

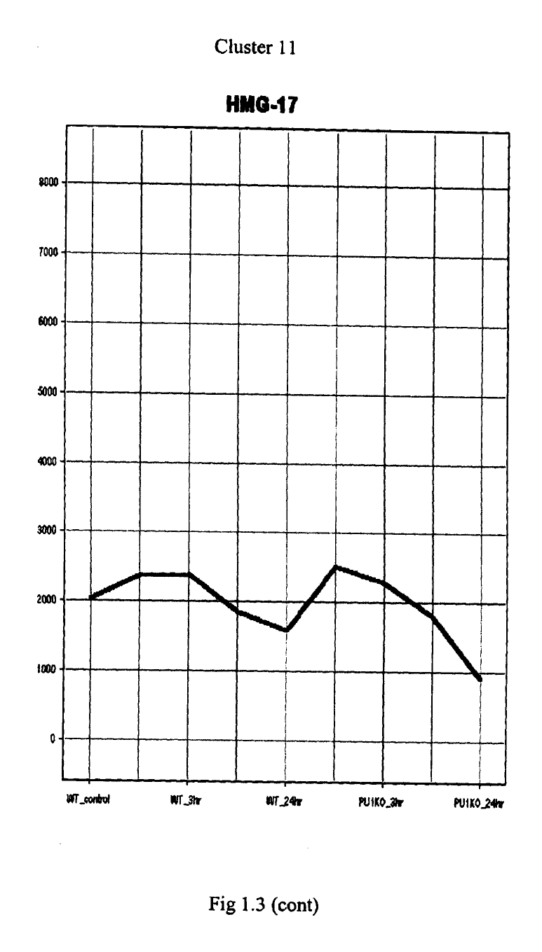

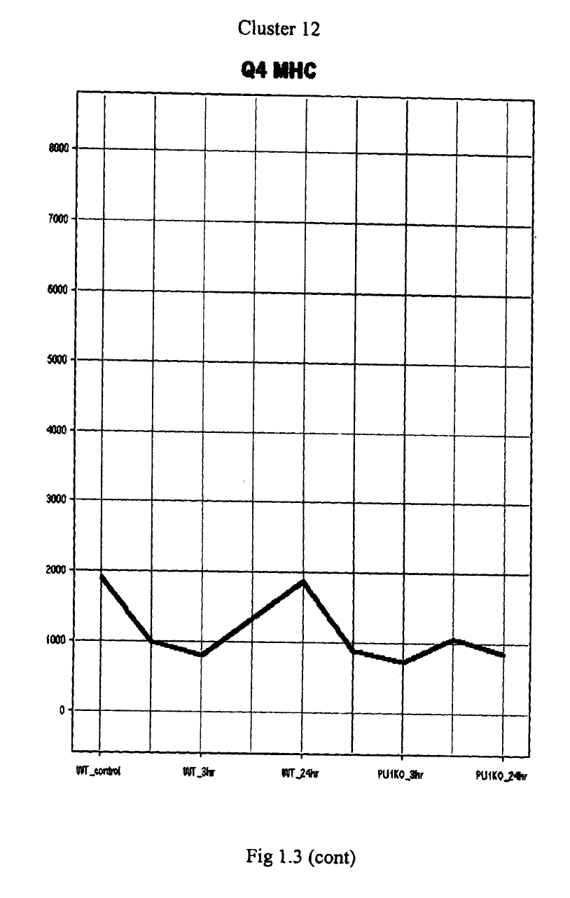

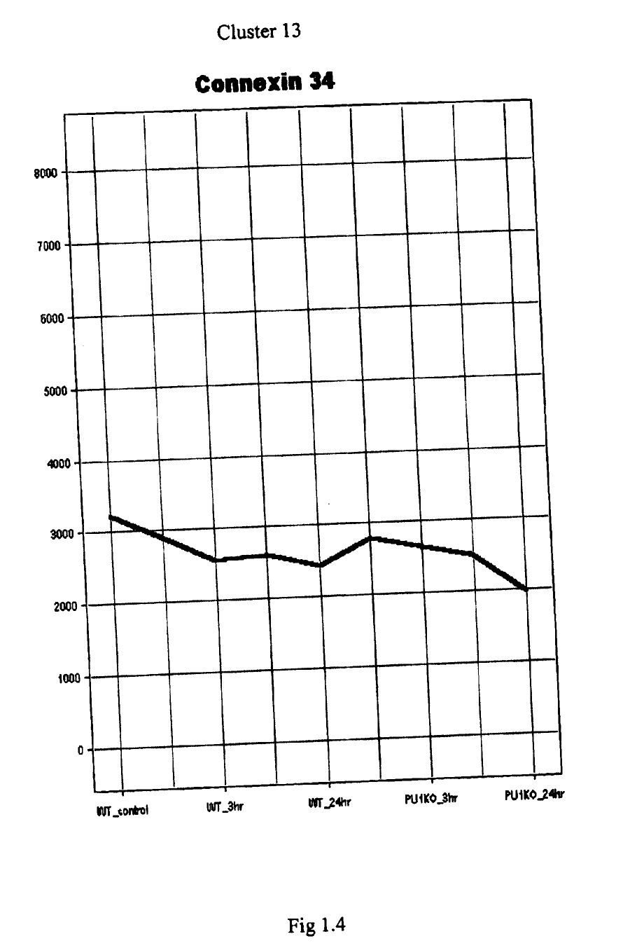

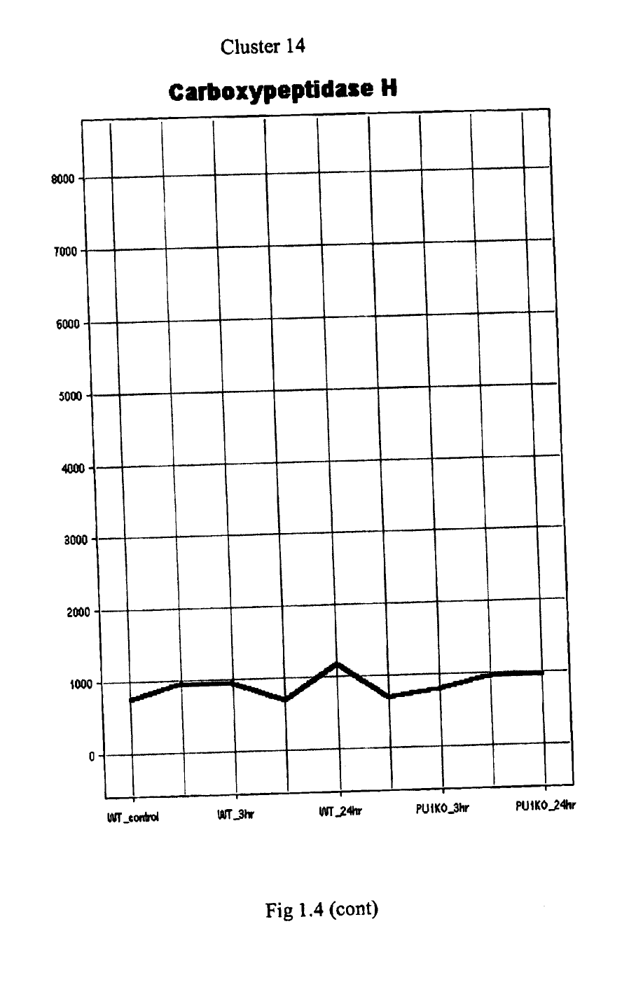

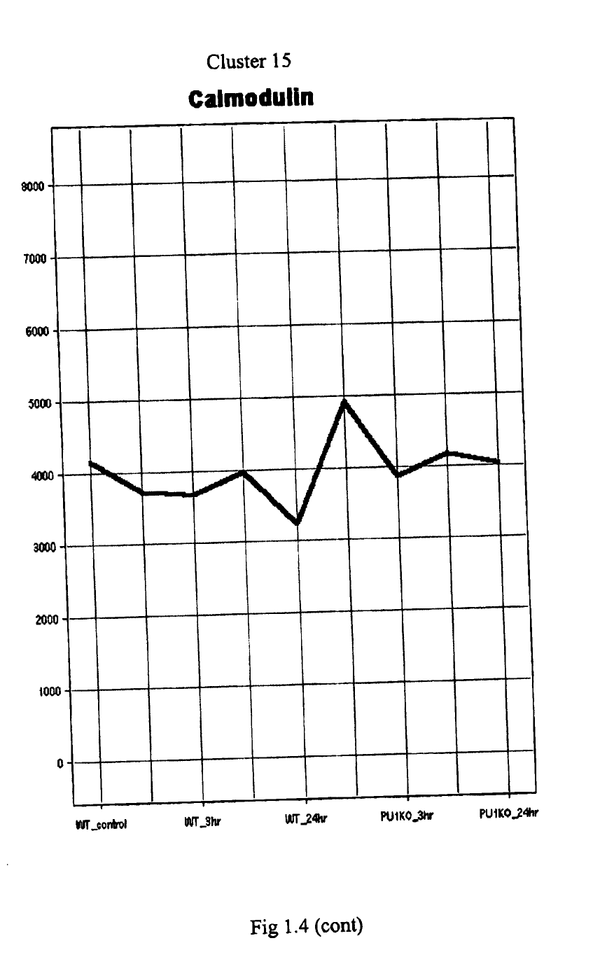

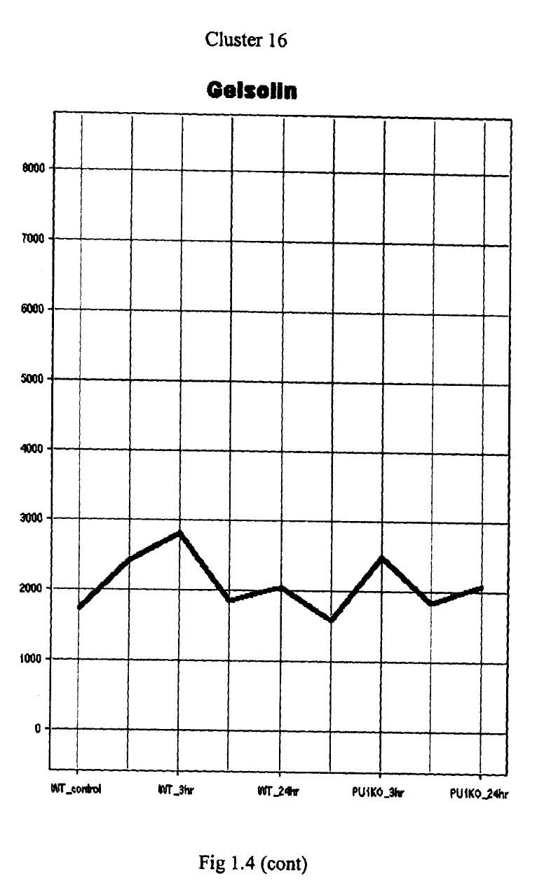

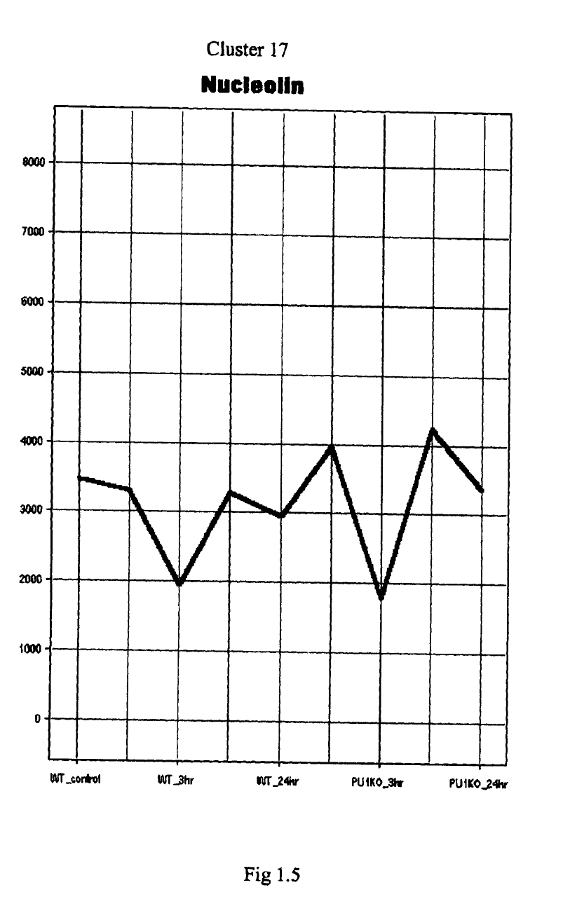

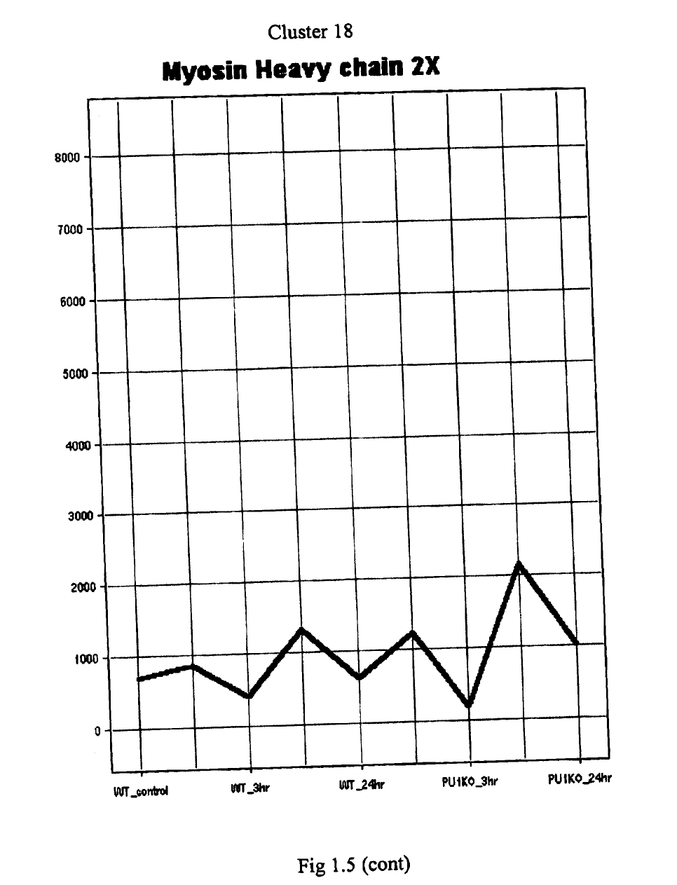

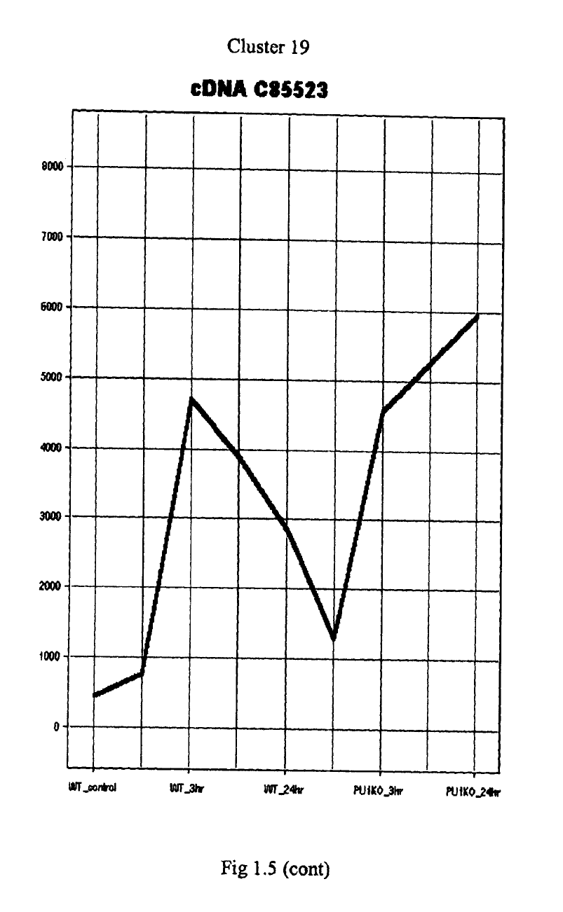

FIG. 1: Profiles of clusters 1-20

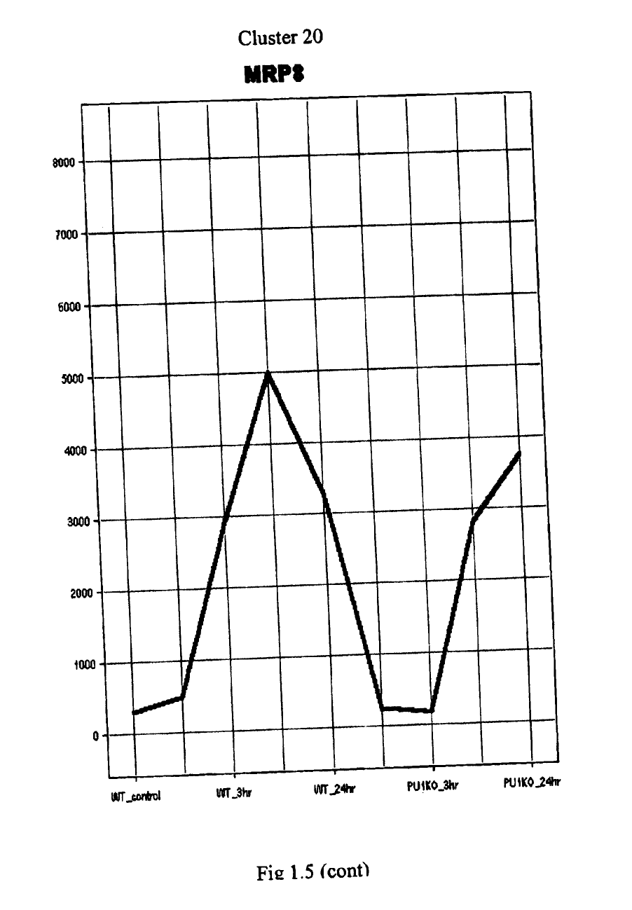

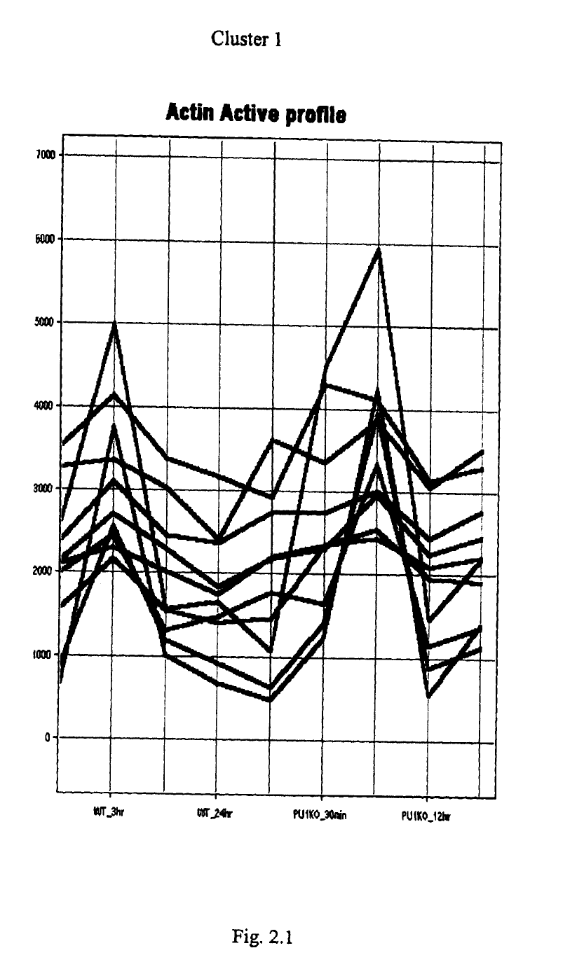

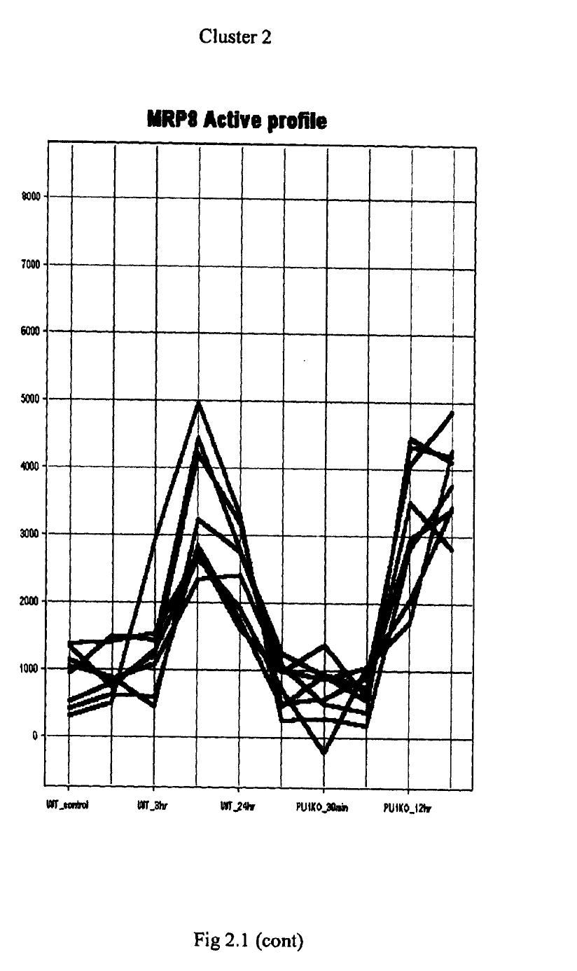

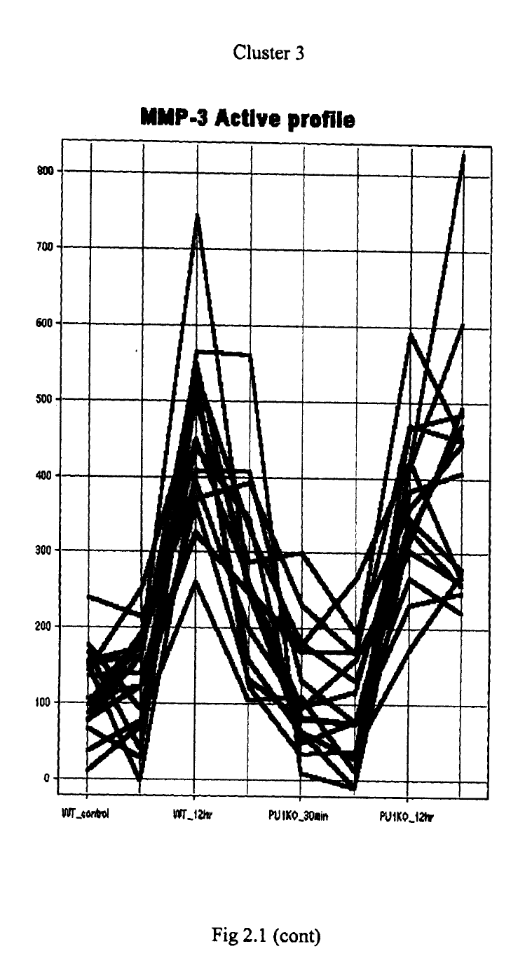

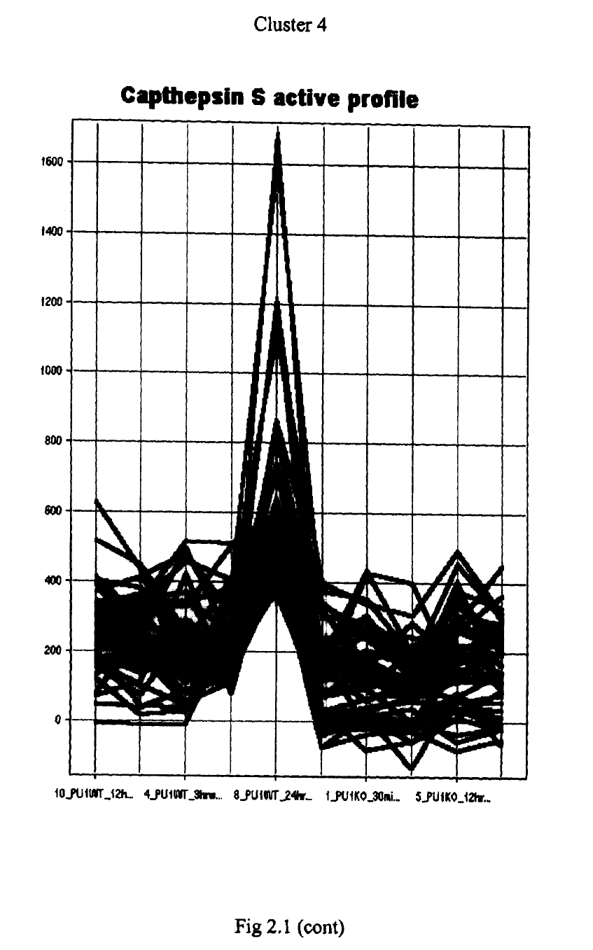

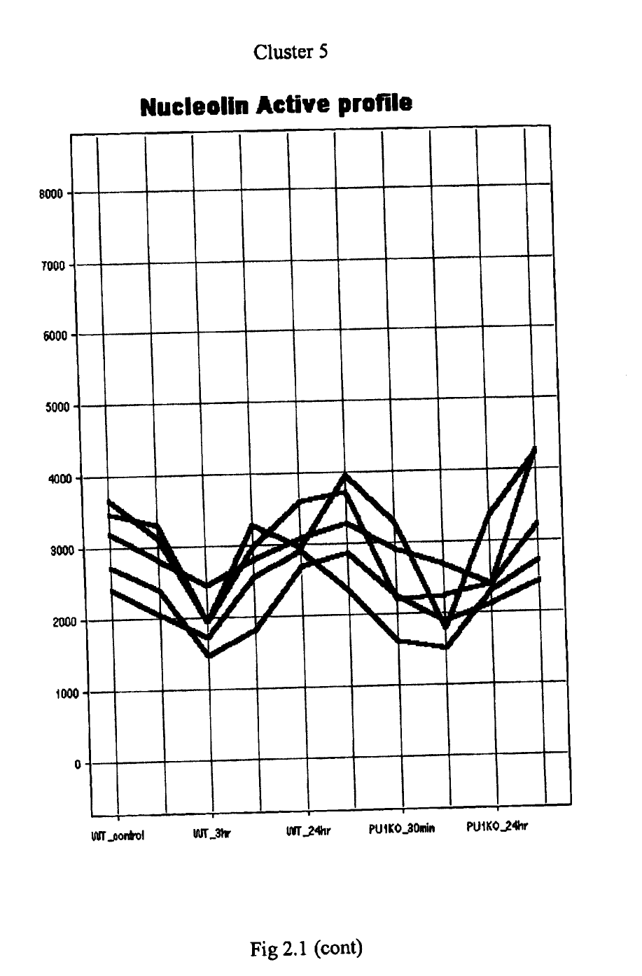

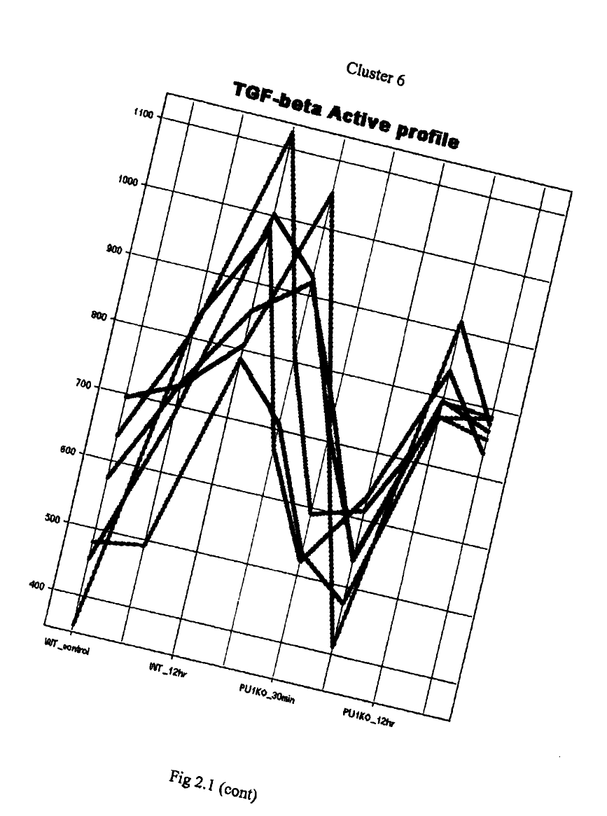

FIG. 2: Profiles of clusters using specific interesting genes as active profiles

### Example 1

Wounding

Ten horizontal and eight vertical incisional wounds were made to a marked area of 0.5 cm×1 cm on a day 2-neonatal mouse back skin. The pups were left with their mothers for the wounds to heal. After 30 minutes, 3 hours, 12 hours and 24 hours, the skin was removed from the back and immediately immersed in liquid nitrogen. A control of uninjured skin was taken at 12 hours.

The mice used for this experiment were either wildtype or PU.1 knockout (KO) mice, the latter are unable to produce inflammatory cells and therefore cannot mount an inflammatory response.

### Example 2

RNA Extraction

The tissue was homogenised using a glass teflon homogeniser. The RNA was extracted using RNAzol (Biogenesis), following the manufacturer's protocol. The RNA was cleaned up using RNeasy minicolumns from Qiagen, following the manufacturer's protocol.

### Example 3

Affymetrix Gene Expression Analysis

Double stranded cDNA was generated from 10 μg total RNA (produced according to example 2) using Superscript Choice kit (Life Technologies) with a T7-polyT primer. Approximately 1 μg cDNA was used to generate biotinylated cRNA by in vitro transcription using Bioarray High Yield RNA Transcript Labeling Kit (Enzo). 10 μg fragmented cRNA was hybridised in 100 mM MES, 1M Na⁺, 20 mM EDTA, 0.01% Tween 20, 0.1 mg/ml herring sperm DNA, 0.5 mg/ml acetylated BSA plus 50 pM control oligonucleotide and eukaryotic hybridisation controls to MGU74A arrays (Affymetrix) at 45° C. for 16 hours. Arrays were washed using Affymetrix protocols in non-stringent buffer (6×SSPE, 0.01% Tween 20, 0.005% antifoam) at 25° C. and stringent wash buffer (100 mM MES, 0.1 M Na⁺, 0.01% Tween) at 50° C. and stained with streptavidin phycoerythrin (10 μg/ml) including an antibody amplification step. Arrays were scanned using a laser confocal scanner to generate fluorescence intensities. The data were analysed using Microarray Analysis Suite version 4.0 (Affymetrix). The data were scaled to a target intensity of 300.

### Example 4

Data Analysis

The results were firstly analysed to remove bad probe sets and to remove those sequences that remained absent during the course of the experiment. This reduced the set to just under 5000 genes. The data for this set were then analysed using Spotfire Array Explorer 3.0 software (see e.g. U.S. Pat. No. 6,014,661).

A non-hierarchical clustering method called K-means clustering was used for the analysis. This is an iterative process with each profile assigned to the closest centroid (centroids are the centre point of the profile cluster). New centroids are calculated for the resulting cluster and the profiles are reassigned to the closest centroid. These steps are repeated until a steady state has been reached. In this instance, the method used to generate the centroid was evenly spaced profiles, i.e. profiles in this case are evenly distributed between the maximum and minimum value for each variable in the profile set. The number of clusters was set to 20, assigning the number of clusters expected should be based on an assessment of the distribution of the data. Similarity to the active profile was calculated using Euclidean distances. It is important to ensure that the cluster number chosen does not produce empty clusters or clusters which contain only one profile. The clusters produced by this method resulted in 20 clusters, with a reasonable number of profiles in each (lowest number of profiles: cluster 10 with 141 profiles, highest number: cluster 3 with 337 profiles). The whole set, ordered by cluster, is shown in Table 1 (found at the end of the example section), the summary profile for each cluster is shown in FIG. 1. The clusters are presented as the time course of expression for the respective group of genes in the order wild type control (taken at 12 hours), wild type 30 minutes after wounding, wild type 3 hours after wounding, wild type 12 hours after wounding, wild type 24 hours after wounding, followed by the same time points for the PU.1 KO mice along the X-axis; the Y-axis indicates level of expression (as measured by fluorescence intensity).

These twenty clusters can be further classified into five types:

1. Essential wound healing genes show increased expression during wound healing in both wild type and PU.1 KO mouse wounds. These include the genes found in Cluster 2, Cluster 7, Cluster 11, Cluster 16 and Cluster 20.

2. Genes associated with the inflammatory response, these are found to be upregulated in the wild type mouse, but not in the PU.1 knock out mouse and therefore are not required for wound healing, the relevant genes are found in clusters 5 and 6.

3. Obligatory down-regulated genes. Genes for which expression is downregulated during wound healing in both wild type and PU.1 KO mouse wounds. These include the genes found in Clusters 4, Cluster 13 and Cluster 17.

4. Wound healing genes that are suppressed by inflammation. Genes in this group show increased expression in both genotypes but the level of gene expression is reduced in wildtype in comparison with the PU.1 KO mice. These genes are found in and Cluster 19 and Cluster 3.

5. Genes that are co-expressed with TGF-β and are therefore implicated in scarring and fibrosis.

1. Essential Wound Healing Genes

These are found in Cluster 2, Cluster 7, Cluster 11, Cluster 16 and Cluster 20.

Cluster 2: 310 Profiles

The profile of this cluster identifies genes whose expression peaks 3 hrs after wounding in both the wild type and the PU.1 KO mice. Examples of prototypical wound healing genes in cluster 2 are transcription factor JunB (immediate early gene, involved in transcriptional regulation) and actin which is required for cell migration. All genes in this cluster are likely to be essential wound healing genes, and/or marker genes indicative of wound repair. Specifically, the nine genes that show a profile most similar to actin, identified using actin as the active profile, are likely to be important (FIG. 2a, Table 2). One or more of the proteins encoded by nucleotide sequences comprising the sequences of the accession numbers grouped in clusters 2, 7, 11, 16, and 20 as indicated in Table 1 can be used as a marker, or markers indicative of wound repair. An inhibitor or antagonist of a protein encoded by a gene comprising a sequence of one of the accession numbers grouped in clusters 2, 7, 11, 16, and 20 can be used for the treatment of a disease or disorder characterised by an excessive healing response, such as, but not limited to, scarring, fibrosis, restenosis post angioplasty, psoriasis, post-traumatic/surgical adhesions of the peritoneal cavity, joints and ligaments, benign prostatic hyperplasia, glaucoma, and peripheral nerve injury. An antisense oligonucleotide, or ribozyme against, or compounds that downregulate transcription of, any of the genes comprising the sequences of the accession numbers grouped in clusters 2, 7, 11, 16, and 20 can be used for the treatment of diseases and disorders characterised by an excessive healing response, such as, but not limited to, scarring, fibrosis, restenosis post angioplasty, psoriasis, post-traumatic/surgical adhesions of the peritoneal cavity, joints and ligaments, benign prostatic hyperplasia, glaucoma, and peripheral nerve injury.

An increase in activity of a protein encoded by a gene comprising a sequence of one of the accession numbers grouped in clusters 2, 7, 11, 16, and 20 can be achieved by, for example, agonists or stimulators of the protein, gene therapy, application of the protein itself, or substances that upregulate the transcription and/or translation of these genes, means that provide an increase in activity of the protein have application in the treatment of diseases and disorders characterised by impaired healing response such as chronic dermal ulcers, oral mucocystis, emphysema, ulcerative diseases of the gastrointestinal (GI) tract, cystitis.

Cluster 7: 218 Profiles

The profile of this cluster identifies genes whose expression peaks early after wounding in both the WT and KO mice.

Importantly, the Connective Tissue Growth Factor Gene Family member WISP-1 is in this cluster and therefore identified as an early regulator of wound repair. WISP-1, or a viral or plasmid construct comprising the coding sequence of WISP-1 under control of a promoter active in mammalian cells is useful for the treatment of conditions or disorders involving impaired wound repair.

Cluster 11: 266 Profiles

The profile of this cluster identifies genes whose expression increases during wound repair in both the wildtype and the PU.1 KO mice. PAF acetyl hydrolase is present in this cluster and therefore identified as a marker of wound repair. As this enzyme is required to deactivate the lipid mediator PAF during inflammation, inhibitors of PAF find use as wound healing agents. PAF antagonists are therefore useful as agents for the treatment of diseases and disorders characterised by impaired healing response. Suitable PAF antagonists are disclosed in European Patent EP 0310386, especially modipafant, which is (+)-4-(2-chlorophenyl)-1,4-dihydro-3-ethoxycarbonyl-6-methyl-2-[4-(2-methylimidazo[4,5-c]pyrid-1-yl)phenyl]-5-(N-[2-pyridyl]carbamoyl)pyridine, and is disclosed in European Patent 0310386 [see Example 34(d)].

Cluster 16: 213 Profiles

The profile of this cluster also identifies genes whose expression peaks early after wounding in both the wildtype and the PU.1 KO mice. Interestingly, cyclooxygenase I is present in this cluster and is a marker of wound repair, particularly early wound repair processes. This enzyme is required to produce bioactive eicosanoids, therefore cyclooxygenase I inhibitors are useful for the treatment of diseases and disorders characterised by excessive healing response.

Cluster 20: 265 Profiles

Genes in this cluster are essential wound healing genes, expressed toward the end of the healing process. These genes are involved in cessation of the healing response, and maturation and remodelling of the repaired tissue, transcription and translation products of these genes are markers for advanced wound repair. MRP-8, a cytokine from the S100 family, previously identified as being strongly induced in normal wounds, is a member of this cluster, and can therefore serve as a marker of advanced wound repair. Eight genes were identified using MRP-8 as the active profile (see FIG. 2b, Table 3). These eight genes and their transcription or translation products are useful as markers of advanced wound repair. EST AA689670 was identified as a keratinocyte marker of advanced wound repair. The presence of MMP3 in this cluster suggests that inhibitors of MMP-3 will be useful to inhibit wound remodelling. Inhibitors of the sixteen gene products that are identified using MMP3 as the active profile will also find use for inhibition of wound remodelling (FIG. 2c, Table 4).

2. Inflammatory Genes not Required for Wound Healing: These are Genes that are Found Upregulated in the Wildtype but not in the PU.1 KO Mice. They are Found in Clusters 5 and 6:

Cluster 5: 321 Profiles, and Cluster 6: 213 Profiles

Genes in these clusters that are not expressed in the PU.1 KO mice represent inflammatory genes not required for wound healing. As excessive inflammation is associated with impaired healing and excessive scarring, these non-essential genes represent targets for the treatment of all types of impaired healing. Antagonists or inhibitors for the products of any of the genes of these clusters are likely to be useful for improving wound healing. Also, genes in these clusters and their transcription and translation products are useful as markers for wound inflammation. Using cathepsin-S as the active profile, 47 genes were found to cluster with cathepsin-S (FIG. 2d, Table 5); these are especially useful as markers for wound inflammation, as well as for screening for inhibitor or antagonist compounds useful for the treatment of impaired healing and excessive scarring. The IMAGE clone number 1429340 is also an example of such an inflammatory marker or potential target gene. Especially inhibitors of ERK2 map kinase will be useful for the treatment of diseases and disorders characterised by impaired healing response.

3. Obligatory Down-regulated Genes.

These include the genes found in Clusters 4 (247 profiles), Cluster 13 (234 profiles) and Cluster 17 (238 profiles).

These clusters represent genes that are down regulated during healing, in both wildtype and PU.1 KO mice. This down regulation may be required for repair to occur. Therefore inhibitors of these genes will be useful to promote wound healing and activators of these genes to prevent overhealing. Apolipoprotein E is found within this cluster and thus compounds that modulate the Apolipoprotein E expression or activity will be useful as agents for the control of wound repair.

3. Genes Required for Wound Healing Which are Suppressed by Inflammation:

Cluster 19: 187 Profiles

This cluster represents essential wound healing genes that show decreased expression during the late macrophage dependent inflammatory response. As excessive inflammation is associated with impaired healing and excessive scarring, these genes, their transcription and translation products are targets for the treatment of impaired healing and excessive scarring. Focal adhesion kinase is found in this cluster and thus inhibitors of focal adhesion kinase can be used to treat treat diseases and disorders of over-repair. Also, the endogenous plasminogen activator inhibitor, PAI-1 is found to be down-regulated by inflammation, therefore inhibitors of Plasminogen activators (especially Urokinase-type plasminogen activator) can be used for the treatment of inflammation-impaired wound healing.

Cluster 3: 337 Profiles

This cluster also contains early wound healing genes that are suppressed by resident inflammatory cells such as mast cells and dendritic cells. Genes in this cluster include fibronectin, which is known to be the earliest scaffold for wound repair. Agents which overcome the resident inflammatory cell suppression of fibronectin synthesis will be useful for the treatment of impaired wound healing. More specifically, agonists of the fibroblast growth factor (FGF) receptor and agonists of the ROR alpha 1 nuclear hormone receptor can be used for the treatment of inflammation impaired wound healing.

5. Genes Involved in Scarring.

TGF-β is known to be a major factor in governing scarring and fibrosis, using TGF-β as the active profile, a cluster of six genes was identified (FIG. 2e, Table 6) each of which thus is implicated in scarring and fibrosis.

Various means to decrease the activity and/or amount of the expression products of these genes can be used for the treatment of diseases and disorders characterised by excessive healing response such as scarring, fibrosis, restenosis post angioplasty, psoriasis, post-traumatic/surgical adhesions of the peritoneal cavity, joints and ligaments, benign prostatic hyperplasia, glaucoma, and peripheral nerve injury. Such means include an inhibitor or antagonist of, or antibody to, any one of the proteins encoded by a gene comprising the sequence of one of the accession numbers clustered with TGF-β; or an antisense oligonucleotide, or ribozyme against, or a compound that downregulates transcription and or translation of, any of the genes comprising the sequence of one of the accession numbers clustered with TGF-β.

Further non-limiting examples are found in attachment 1.

### Example 5

Delivery, Wounding and Culture of E11.5 Mouse Embryos

Embryonic day 11.5 (E11.5) embryos were obtained by means of timed matings of CD1 mice, with embryos considered to be E0.5 at noon on the day of plug checking. E11.5 embryos were dissected from the uterus in Tyrode's saline and prepared for culture as described by P Martin & D Cockcroft, (1999) Culture of post-implantation mouse embryos, in Molecular embryology methods and protocols (editors Sharpe, P and Mason, I). New York; Humana Press p7-22. Standard wounding was performed by amputating the left hindlimb at its base using iridectomy scissors as described by McCluskey and Martin (1995) Developmental Biology, 17: 102-114. Using an electrolytically sharpened tungsten needle, incisional wounds of approximately 500 μm in length were also generated on the left forelimb, as well as superficial puncture wounds on the flank. Wounded embryos were transferred to 50 ml Falcon tubes containing 4 ml of culture medium (3 ml of Tyrode's saline plus 1 ml of normal rat serum) and roller-cultured for up to 24 hours in an atmosphere of 95% oxygen/5% carbon dioxide at 30 rpm and 37° C. (Martin and Cockcroft (1999), as above).

### Example 6

Frozen Section in Situ Hybridisation of Wound Tissue

Sections were hybridised (as described by DG Wilkinson & MA Nieto (1993) Methods Enzymol 225: 361-373) at 55° C. overnight with digoxigenin labelled antisense radioprobes specific for the gene of interest. Expression was visualised by BCIP/NBT precipitation (Boehringer Mannheim) and sections were viewed on a Zeiss Axiophot microscope, after mounting in Citifluor (UKC). The labelled antisense radioprobe biomarkers derived from the gene of interest demonstrate expression of the gene of interest.

EST 1 of attachment 1 is identified by accession number AA689670, this sequence corresponds to SEQ ID No. 32; the gene comprising this sequence is found in cluster AA689670 (SEQ ID No. 32) is a keratinocyte marker of advanced wound repair. Biomarkers derived from the gene comprising SEQ ID NO. 32 are useful as markers of advanced wound repair. In situ hybridisation on 24 hour frozen wound sections using an antisense radioprobe biomarker derived from the gene shows that this gene is expressed in the basal layers of the epidermis and expression extends back 10 to 12 cell diameters from the wound site.

EST 3 of attachment 1 is identified by accession number A1153421, this sequence corresponds to SEQ ID No. 19; the gene comprising this sequence is found in cluster 5. Al153421 (SEQ ID No. 19) is a biomarker for wound inflammation. Biomarkers derived from the gene comprising SEQ ID No. 19 are useful as markers of wound inflammation and/or impaired healing. In situ hybridisation on 24 hour frozen wound tissue sections using an antisense radioprobe biomarker derived from the gene shows that this gene is expressed in all tissue layers by cells as part of the inflammatory response downstream of macrophage infiltration.

| | Affymetrix | | | | | | | | |
|---|---|---|---|---|---|---|---|---|---|
| | accession | | WT | | WT 30 | | WT 3 | | WT 12 |
| Cluster | number | Description | control | Call | min | Call | hours | Call | hours |
| 1 | 92769_at | Cluster Incl M25149: Tissue specific transplantation antigen P91A/cds = (0,1589)/gb = M25149/gi = 191974/ug = Mm 12194/ | 869.2 | P | 996.9 | P | 1016.8 | P | 844.2 |
| | | len = 1590/STRA = for | | | | | | | |
| 1 | 93565_at | Cluster Incl AF055665: Kinesin light chain 1/cds = (94,1719)/gb = AF055665/gi = 3347845/ug = Mm 20354/len = 2289/ | 217.1 | P | 287 | P | 203 | P | 215.3 |
| | | STRA = for | | | | | | | |
| 1 | 93589_at | Cluster Incl AI851172: UI-M-BH0-akg-g-03-0-UI s1 Mus musculus cDNA, 3 end/clone = UI-M-BH0-akg-g-03-0-UI/ | 842.8 | P | 862.4 | P | 896.6 | P | 820.5 |
| | | clone_end = 3/gb = AIB51172/gi = 5495043/ug = Mm 20806/len = 363/STRA = for | | | | | | | |
| 1 | 93749_at | Cluster Incl AI848045: UI-M-AH1-agp-g-05-0-UI s1 Mus musculus cDNA, 3 end/clone = UI-M-AH1-agp-g-05-0-UI/ | 168.5 | P | 176 | P | 167.9 | P | 217.7 |
| | | clone_end = 3/gb = AI848045/gi = 5491951/ug = Mm 21108/len = 224/STRA = for | | | | | | | |
| 1 | 93757_at | Cluster Incl AI843574 UI-M-AO1-aen-d-12-0-UI s1 Mus musculus cDNA, 3 end/clone = UI-M-AO1-aen-d-12-0-UI/ | 1399 | P | 1542.8 | P | 1353.2 | P | 1475.7 |
| | | clone_end = 3/gb = AI843574/gi = 5477827/ug = Mm 21132/len = 352/STRA = for | | | | | | | |
| 1 | 94024_at | Cluster Incl AA671429 vn81g12 r1 Mus musculus cDNA, 5 end/clone = IMAGE-1038406/clone_end = 5/gb = AA671429/ | 935.2 | P | 868.8 | P | 880.6 | P | 834.4 |
| | | gi = 2643508/ug = Mm 21873/len = 464/STRA = for | | | | | | | |
| 1 | 94254_at | Cluster Incl AI845237 UI-M-BG0-aht-g-09-0-UI s1 Mus musculus cDNA, 3 end/clone = UI-M-BG0-aht-g-09-0-UI/ | 1201.3 | P | 986.3 | P | 1318.3 | P | 1370.4 |
| | | clone_end = 3/gb = AI845237/gi = 5489143/ug = Mm 22409/len = 255/STRA = for | | | | | | | |
| 1 | 94898_at | Cluster Incl AI848555 UI-M-AP1-agf-h-08-0-UI.s2 Mus musculus cDNA, 3 end/clone = UI-M-AP1-agf-h-08-0-UI/ | 289 | P | 257.5 | P | 300.4 | P | 261.7 |
| | | clone_end = 3/gb = AI848555/gi = 5492461/ug = Mm 24015/len = 426/STRA = for | | | | | | | |
| 1 | 96001_at | Cluster Incl AW122293 UI-M-BH2 2-aos-c-01-0-UI s1 Mus musculus cDNA, 3 end/clone = UI-M-BH2 2-aos-c-01-0-UI/ | 1078.7 | P | 1061.8 | P | 1158.5 | P | 981.7 |
| | | clone_end = 3/gb = AW122293/gi = 6097778/ug = Mm 25321/len = 363/STRA = for | | | | | | | |
| 1 | 96045_at | Cluster Incl AW050142 UI-M-BH1-ano-e-11-0-UI.s1 Mus musculus cDNA, 3 end/clone = UI-M-BH1-ano-e-11-0-UI/ | 275.4 | P | 194.2 | P | 256.9 | P | 202.8 |
| | | clone_end = 3/gb = AW050142/gi = 5910671/ug = Mm 25994/len = 458/STRA = for | | | | | | | |
| 1 | 96672_at | Cluster Incl AW123564 UI-M-BH2.1-aqb-h-01-0-UI.s1 Mus musculus cDNA, 3 end/clone = UI-M-BH2.1-aqb-h-01-0-UI/ | 2099.7 | P | 1733.1 | P | 2023.3 | P | 2475.9 |
| | | clone_end = 3/gb = AW123564/gi = 6099094/ug = Mm 27401/len = 365/STRA = for | | | | | | | |
| 1 | 96716_at | Cluster Incl AW121102: UI-M-BH2 3-aoh-b-01-0-UI s1 Mus musculus cDNA, 3 end/clone = UI-M-BH2 3-aoh-b-01-0-UI/ | 686.7 | P | 586.9 | P | 637.2 | P | 505.9 |
| | | clone_end = 3/gb = AW121102/gi = 6096435/ug = Mm 27647/len = 353/STRA = rev | | | | | | | |
| 1 | 97226_at | Cluster Incl AW125649: UI-M-BH2.2-aqi-b-03-0-UI s1 Mus musculus cDNA, 3 end/clone = UI-M-BH2 2-aqi-b-03-0-UI/ | 382.7 | P | 415.8 | P | 394.7 | P | 372.8 |
| | | clone_end = 3/gb = AW125649/gi = 6101179/ug = Mm 27773/len = 435/STRA = for | | | | | | | |
| 1 | 97263_s_at | Clusler Incl AI846289 UI-M-AK1-aez-f-02-0-UI s1 Mus musculus cDNA, 3 end/clone = UI-M-AK1-aez-f-02-0-UI/ | 1047.8 | P | 1023.6 | P | 1373.6 | P | 1214.5 |
| | | clone_end = 3/gb = AI846289/gi = 5490195/ug = Mm 27899/len = 375/STRA = for | | | | | | | |
| 1 | 97284_at | Clusler Incl AI853789 UI-M-BH0-aig-f-10-0-UI s1 Mus musculus cDNA, 3 end/clone = UI-M-BH0-aig-f-10-0-UI/ | 694.2 | P | 690.6 | P | 671.2 | P | 649.6 |
| | | clone_end = 3/gb = AI853789/gi = 5497695/ug = Mm 27990/len = 458/STRA = for | | | | | | | |
| 1 | 97817_at | Cluster Incl AW121136 UI-M-BH2 3-aoh-e-05-0-UI s1 Mus musculus cDNA, 3 end/clone = UI-M-BH2 3-aoh-e-05-0-UI/ | 776.6 | P | 904.7 | P | 833.2 | P | 858 |
| | | clone_end = 3/gb = AW121136/gi = 6096469/ug = Mm 28189/len = 391/STRA = for | | | | | | | |
| 1 | 97823_g_at | Cluster Incl AW122689 UI-M-BH2 2-aot-a-10-0-UI s1 Mus musculus cDNA, 3 end/clone = UI-M-BH2 2-aot-a-10-0-UI/ | 757.8 | P | 685.2 | P | 787 | P | 754.5 |
| | | clone_end = 3/gb = AW122689/gi = 6098219/ug = Mm 28202/len = 467/STRA = rev | | | | | | | |
| 1 | 97871_at | Cluster Incl AW120614 UI-M-BH2 3-anx-g-02-0-UI s1 Mus musculus cDNA, 3 end/clone = UI-M-BH2 3-anx-g-02-0-UI/ | 458.8 | P | 332.5 | P | 340.9 | P | 368.8 |
| | | clone_end = 3/gb = AW120614/gi = 6095947/ug = Mm.28339/len = 294/STRA = rev | | | | | | | |
| 1 | 98882_s_at | Cluster Incl AW047320 UI-M-BH1-amh-g-06-0-UI s1 Mus musculus cDNA, 3 end/clone = UI-M-BH1-amh-g-06-0-UI/ | 1600 | P | 1322.8 | P | 1410.1 | P | 1498.2 |
| | | clone_end = 3/gb = AW047320/gi = 5907849/ug = Mm.28527/len = 616/STRA = for | | | | | | | |
| 1 | 94451_at | Cluster Incl AI787627 UI20f06 y1 Mus musculus cDNA, 5 end/clone = IMAGE-2088131/clone_end = 5/gb = AI787627/ | 704.2 | P | 604.5 | P | 650.8 | P | 608.2 |
| | | gi = 5335343/ug = Mm 28786/len = 450/STRA = for | | | | | | | |
| 1 | 95666_at | Cluster Incl AW123404 UI-M-BH2 1-apj-a-09-0-UI s1 Mus musculus cDNA, 3 end/clone = UI-M-BH2 1-apj-a-09-0-UI/ | 413.3 | P | 540.7 | P | 397.7 | P | 330.7 |
| | | clone_end = 3/gb = AW123404/gi = 6098934/ug = Mm 29572/len = 354/STRA = for | | | | | | | |
| 1 | 95756_at | Clusler Incl AI839681: UI-M-AN0-acp-d-02-0-UI s1 Mus musculus cDNA, 3 end/clone = UI-M-AN0-acp-d-02-0-UI/ | 511.3 | P | 447.2 | P | 455.9 | P | 444.1 |
| | | clone_end = 3/gb = AI839681/gi = 5473894/ug = Mm 29795/len = 348/STRA = for | | | | | | | |
| 1 | 96301_at | Clusler Incl AI851912 UI-M-BH0-aix-e-03-0-UI s1 Mus musculus cDNA, 3 end/clone = UI-M-BH0-aix-e-03-0-UI/ | 942.5 | P | 841.7 | P | 963.7 | P | 651.4 |
| | | clone_end = 3/gb = AI851912/gi = 5495818/ug = Mm 29911/len = 392/STRA = for | | | | | | | |
| 1 | 96357_at | Cluster Incl AW212775 uo66e09.x1 Mus musculus cDNA, 3 end/clone = IMAGE-2647528/clone_end = 3/gb = AW212775/ | 479.8 | P | 715.6 | P | 675.2 | P | 801.3 |
| | | gi = 6518896/ug = Mm 30010/len = 165 STRA = for | | | | | | | |
| 1 | 97550_at | Cluster Incl AW047228 UI-M-BH1-amb-f-04-0-UI.s1 Mus musculus cDNA, 3 end/clone = UI-M-BH1-amb-f-04-0-UI/ | 624.9 | P | 703 | P | 708.7 | P | 737.8 |
| | | clone_end = 3/gb = AW047228/gi = 5907757/ug = Mm.33797/len = 314/STRA = for | | | | | | | |
| 1 | 99559_at | Cluster Incl U14390 Aldehyde dehydrogenase 4, liver microsomal (class 3)/cds = (113,1587)/gb = U14390/gi = 687253/ | 1108.1 | P | 726.1 | P | 1006.7 | P | 989.4 |
| | | ug = Mm 4210/len = 2997/STRA = for | | | | | | | |
| 1 | 100566_at | Cluster Incl L12447 Insulin-like growth factor binding protein 5/cds = (387,1202)/gb = L12447/gi = 425405/ug = Mm.578/ | 380.8 | P | 614 | P | 372.4 | P | 491.5 |
| | | len = 5561/STRA = for | | | | | | | |
| 1 | 101490_at | Cluster Incl AW122692 UI-M-BH2 2-aot-b-01-0-UI s1 Mus musculus cDNA, 3 end/clone = UI-M-BH2 2-aol-b-01-0-UI/ | 450.7 | P | 433.3 | P | 591.3 | P | 464.7 |
| | | clone_end = 3/gb = AW122692/gi = 6098222/ug = Mm 7884/len = 288/STRA = for | | | | | | | |
| 1 | 101520_at | Cluster Incl AF032967 Mus musculus strain C3H/HeN mRNA preferentially expressed In LPS-normoresponsive | 305 | P | 303.6 | P | 345.9 | P | 270.7 |
| | | macrophages/cds = UNKNOWN/gb = AF032967/gi = 4103990/ug = Mm.8540/len = 1766/STRA = for | | | | | | | |
| 1 | 101579_at | Clusler Incl X78304: M. musculus (SRP9) signal recognition particle subunit mRNA, 689bp/cds = (93,353)/gb = X78304/ | 495.9 | P | 406.1 | P | 491.7 | P | 483.7 |
| | | gi = 872130/ug = Mm 89927/len = 689/STRA = for | | | | | | | |
| 1 | 102324_at | Cluster Incl AF036875 Mus musculus multiple endocrine neoplasia type 1 candidate prolein number 18 mRNA, complete | 288.5 | P | 199.3 | P | 184.1 | P | 141.4 |
| | | cds/cds = (19,753)/gb = AF036875/gi = 3136115/ug = Mm 10721/len = 841/STRA = for | | | | | | | |
| 1 | 102768_l_at | Cluster Incl AB016248 Mus musculus mRNA for sterol-C5-desaturase, complete cds/cds = (170,1069)/gb = AB016248/ | 123.3 | P | 109.2 | P | 72.7 | P | 123.6 |
| | | gi = 3721883/ug = Mm.13081/len = 1721/STRA = for | | | | | | | |
| 1 | 102993_at | Cluster Incl M85153: Glycoprotein galactosyltransferase alpha 1, 3/cds = (444,1559)/gb = M85153/gi = 193563/ug = Mm.1548/ | 655.5 | P | 723.7 | P | 628.9 | P | 603.8 |
| | | len = 3450/STRA = for | | | | | | | |
| 1 | 103005_s_at | Cluster Incl X66084 CD44 antigen/cds = (9,1976)/gb = X66084/gi = 53679/ug = Mm 15643/len = 1997/STRA = for | 320.1 | P | 206.5 | P | 234.7 | P | 483.4 |
| 1 | 103052_r_at | Cluster Incl X76653 Apolipoprotein regulatory protein 1/cds = (35,1279)/gb = X76653/gi = 482927/ug = Mm.16519/len = 2495/ | 121.2 | P | 124.3 | P | 124.6 | P | 123.4 |
| | | STRA = for | | | | | | | |
| 1 | 103345_at | Cluster Incl AW046708: UI-M-BH1-alt-d-05-0-Ul.s1 Mus musculus cDNA, 3 end/clone = UI-M-BH1-alt-d-05-0-UI/ | 585 | P | 818.6 | P | 672.6 | P | 691.3 |
| | | clone_end = 3/gb = AW046708/gi = 5907277/ug = Mm. 18249/len = 412/STRA = for | | | | | | | |
| 1 | 103669_at | Cluster Incl AA793857: vr40b03.s1 Mus musculus cDNA, 5 end/clone = IMAGE-1123085/clone_end = 5/gb = AA793857/ | 334.5 | P | 401.6 | P | 308.9 | P | 482.7 |
| | | gi = 2856812/ug = Mm.2077/len = 261/STRA = for | | | | | | | |
| 1 | 103814_at | Cluster Incl AF093871.Mus musculus peroxisomal biogenesis factor (Pex11b) mRNA, complete cds/cds = (3,782)/ | 502.3 | P | 486.4 | P | 488 | P | 542.5 |
| | | gb = AF093671/gi = 3907595/ug = Mm 20901/len = 1109/STRA = for | | | | | | | |
| 1 | 103832_at | Cluster Incl AF097181 Mus musculus tuftelin-interacting protein 10 mRNA, complete cds/cds = (96,1676)/gb = AF097181/ | 407.4 | P | 405 | P | 329.5 | P | 318.4 |
| | | gi = 3851163/ug = Mm.20933/len = 2424/STRA = for | | | | | | | |
| 1 | 104069_at | Cluster Incl AI852737 UI-M-BH0-aji-h-05-0-UI.s1 Mus musculus cDNA, 3 end/clone = UI-M-BH0-aji-h-05-0-UI/clone_end = 3/ | 1083.1 | P | 898.4 | P | 927.6 | P | 1019.6 |
| | | gb = AI852737/gi = 5496643/ug = Mm 21619/len = 503/STRA = rev | | | | | | | |
| 1 | 104091_at | Cluster Incl AW122563 UI-M-BH2 2-aox-e-09-0-UI s1 Mus musculus cDNA, 3 end/clone = UI-M-BH2 2-aox-e-09-0-UI/ | 556.2 | P | 580.6 | P | 586.7 | P | 535.5 |
| | | clone_end = 3/gb = AW122563/gi = 6098087/ug = Mm 21814/len = 274/STRA = for | | | | | | | |
| 1 | 104128_at | Cluster Incl AW048075 UI-M-BH1-alq-a-05-0-UI s1 Mus musculus cDNA, 3 end/clone = UI-M-BH1-alq-a-05-0-UI/ | 1078.4 | P | 1030.9 | P | 918.7 | P | 1053.6 |
| | | clone_end = 3/gb = AW048075/gi = 5908604/ug = Mm 22040/len = 340/STRA = for | | | | | | | |
| 1 | 104574_at | Cluster Incl AW210607.um62e04.x1 Mus musculus cDNA, 3 end/clone = IMAGE-2285982/clone_end = 3/gb = AW210607/ | 526.4 | P | 466.8 | P | 540.3 | P | 552 |
| | | gi = 6516547/ug = Mm 23927/len = 418/STRA = rev | | | | | | | |
| 1 | 94973_at | Cluster Incl AA840126 ud01d09 r1 Mus musculus cDNA, 5 end/clone = lMAGE-1433873/clone_end = 5/gb = AA840126/ | 190.3 | P | 222 | P | 183.2 | P | 198.9 |
| | | gi = 2915835/ug = Mm 27232/len = 471/STRA = for | | | | | | | |
| 1 | 95010_at | Cluster Incl U21050 Tnf receptor-associated factor 3/cds = (180,1883)/gb = U21050/gi = 719292/ug = Mm.27431/len = 2359/ | 249.6 | P | 305.9 | P | 239.4 | P | 236.5 |
| | | STRA = for | | | | | | | |
| 1 | 95563_at | Cluster Incl AJ130977 Mus musculus mRNA for Ariadne protein, partial/cds = (0,1394)/gb = AJ130977/gi = 3925718/ | 175.9 | P | 64.8 | P | −38.7 | P | 64.3 |
| | | ug = Mm 27783/len = 1629/STRA = for | | | | | | | |
| 1 | 96183_at | Cluster Incl AW122985: UI-M-BH2 1-apb-b-02-0-UI s1 Mus musculus cDNA, 3 end/clone = UI-M-BH2 1-apb-b-02-0-UI/ | 251.1 | P | 167.4 | P | 250.9 | P | 165.6 |
| | | clone_end = 3/gb = AW122985/gi = 6098515/ug = Mm 28459/len = 427/STRA = for | | | | | | | |
| 1 | 96219_at | Cluster Incl AI850357: UI-M-BG1-aig-b-01-0-UI s1 Mus musculus cDNA. 3 end/clone = UI-M-BG1-aig-b-01-0-UI/ | 967.5 | P | 663.5 | P | 797.8 | P | 868 |
| | | clone_end = 3/gb = AIB50357/gi = 5494263/ug = Mm 28702/len = 456/STRA = for | | | | | | | |
| 1 | 96725_at | Cluster Incl AI838398. UI-M-AJ0-aav-h-02-0-UI s2 Mus musculus cDNA, 3 end/clone = UI-M-AJ0-aav-h-02-0-UI/ | 469.6 | P | 470 | P | 533.9 | P | 551.8 |
| | | clone_end = 3/gb = AI838398/gi = 5472611/ug = Mm 28833/len = 260/STRA = for | | | | | | | |
| 1 | 96749_f_at | Cluster Incl AA619554 vo88e08 r1 Mus musculus cDNA, 5 end/clone = IMAGE-1066214/clone_end = 5/gb = AA619554/ | 41.7 | P | 31.2 | P | 39 | P | 34.6 |
| | | gi = 2523430/ug = Mm 28958/len = 468/STRA = for | | | | | | | |
| 1 | 96832_at | Cluster Incl AA606878 vm87a04 r1 Mus musculus cDNA, 5 end/clone = IMAGE-1005198/clone_end = 5/gb = AA606878/ | 748.2 | P | 967.2 | P | 846 | P | 983.7 |
| | | gi = 2455771/ug = Mm 29419/len = 223/STRA = for | | | | | | | |
| 1 | 97392_at | Cluster Incl AW050086 UI-M-BH1-amm-g-09-0-UI s1 Mus musculus cDNA, 3 end/clone = UI-M-BH1-amm-g-09-0-UI/ | 535.3 | P | 470.2 | P | 461.5 | P | 405.1 |
| | | clone_end = 3/gb = AW050086/gi = 5910615/ug = Mm 29809/len = 442/STRA = for | | | | | | | |
| 1 | 97395_at | Cluster Incl AW122465 UI-M-BH2 2-aou-c-10-0-UI s1 Mus musculus cDNA, 3 end/clone = UI-M-BH2 2-aou-c-10-0-UI/ | 977.7 | P | 978 | P | 1056.2 | P | 1109.2 |
| | | clone_end = 3/gb = AW122465/gi = 6097985/ug = Mm 29835/len = 221/STRA = for | | | | | | | |
| 1 | 97917_at | Cluster Incl Y13778 Mus musculus GCN5L1 gene, exon 1 and joined CDS/cds = (70,447)/gb = Y13778/gi = 2769584/ | 1261.7 | P | 1306.9 | P | 1366 | P | 1244 |
| | | ug = Mm 30118/len = 448/STRA = for | | | | | | | |
| 1 | 97957_at | Cluster Incl AF072759 Mus musculus fatty acid transport protein 4 mRNA, partial cds/cds = (0,1520)/gb = AF072769/ | 442.4 | P | 310.7 | P | 524.4 | P | 511.2 |
| | | gi = 3335568/ug = Mm.30487/len = 1521/STRA = for | | | | | | | |
| 1 | 98439_at | Cluster Incl AW049358 UI-M-BH1-ane-a-03-0-UI s1 Mus musculus cDNA, 3 end/clone = UI-M-BH1-ane-a-03-0-UI/ | 429.2 | P | 393.6 | P | 355.5 | P | 422.2 |
| | | clone_end = 3/gb = AW049356/gi = 5909885/ug = Mm 34490/len = 437/STRA = for | | | | | | | |
| 1 | 99033_at | Cluster Incl AI837130 UI-M-AK0-adc-g-01-0-UI.s1 Mus musculus cDNA, 3 end/clone = UI-M-AK0-adc-g-01-0-UI/ | 1032.7 | P | 1110.5 | P | 1018.4 | P | 1246.1 |
| | | clone_end = 3/gb = AI837130/gi = 5471343/ug = Mm.39038/len = 406/STRA = for | | | | | | | |
| 1 | 99068_at | Cluster Incl X80169 M musculus mRNA for 200 kD protein/cds = (252,6086)/gb = X80169/gi = 562764/ug = Mm 3989/ | 456.7 | P | 404.6 | P | 372.6 | P | 438.8 |
| | | len = 6331/STRA = for | | | | | | | |
| 1 | 99445_at | Cluster Incl AW047012 UI-M-BH1-alp-g-12-0-UI s1 Mus musculus cDNA, 3 end/clone = UI-M-BH1-alp-g-12-0-UI/ | 268.6 | P | 337.7 | P | 420.2 | P | 328.7 |
| | | clone_end = 3/gb = AW047012/gi = 5907541/ug = Mm 4042/len = 373/STRA = for | | | | | | | |
| 1 | 99492_at | Cluster Incl AI851990. UI-M-BH0-aiq-e-01-0-UI.s1 Mus musculus cDNA, 3 end/clone = UI-M-BH0-aiq-e-01-0-UI/ | 492.1 | P | 439.2 | P | 414.7 | P | 435.5 |
| | | clone_end = 3/gb = AI851990/gi = 5495896/ug = Mm.41550/len = 404/STRA = for | | | | | | | |
| 1 | 100010_at | Cluster Incl U36340 Mus musculus CACCC-box binding protein BKLF mRNA, complete cds/cds = (44,1078)/gb = U36340/ | 447.3 | P | 213.5 | P | 349.1 | P | 358 |
| | | gi = 1244514/ug = Mm 4549/len = 1200/STRA = for | | | | | | | |
| 1 | 101837_g_at | Cluster Incl D45859 Protein phosphatase 1B, magnesium dependent, beta isoform/cds = (0,461)/gb = D45859/gi = 961469/ | 334.2 | P | 215.4 | P | 310.8 | P | 291.9 |
| | | ug = Mm 849/len = 546/STRA = for | | | | | | | |
| 1 | 101858_at | Cluster Incl AF015770 Mus musculus radical Innge (radical-fringe) mRNA, complete cds/cds = (0,998)/gb = AF015770/ | 635.3 | P | 614.5 | P | 584.8 | P | 598 |
| | | gi = 2454573/ug = Mm 871/len = 999/STRA = for | | | | | | | |
| 1 | 101933_at | Cluster Incl AF035646 Mus musculus small GTPase Rab10 (Rab10) mRNA, complete cds/cds = (241,843)/gb = AF035646/ | 670.2 | P | 562.8 | P | 517.6 | P | 582.3 |
| | | gi = 3406427/ug = Mm 9455/len = 897/STRA = for | | | | | | | |
| 1 | 101938_at | Cluster Incl X75959 M. musculus mRNA for polyA binding protein (testis-enriched isoform)/cds = (163,2049)/gb = X75959/ | 322.1 | P | 349.5 | P | 334.7 | P | 343.2 |
| | | gi = 473091/ug = Mm 9493/len = 2135/STRA = for | | | | | | | |
| 1 | 102306_at | Cluster Incl AF060178 Mus musculus heparan sulfate 2-sulfotransferase (Hs2st) mRNA, complete cds/cds = (37,1107)/ | 254.8 | P | 229.6 | P | 233.2 | P | 246 |
| | | gb = AF060178/gi = 3228535/ug = Mm 12863/len = 2057/STRA = for | | | | | | | |
| 1 | 102960_at | Cluster Incl X96618 M. musculus mRNA for novel stromal cell protein/cds = (96,761)/gb = X96618/gi = 1370221/ | 344.1 | P | 350.5 | P | 207.5 | P | 288.9 |
| | | ug = Mm.17958/len = 1031/STRA = for | | | | | | | |
| 1 | 103219_at | Cluster Incl AI327518: mb43a09 y1 Mus musculus cDNA, 5 end/clone = IMAGE-332152/clone_end = 5/gb = AI327518/ | 451.7 | P | 708.7 | P | 634.9 | P | 631.3 |
| | | gi = 4061947/ug = Mm 19941/len = 423/STRA = for | | | | | | | |
| 1 | 103439_at | Cluster Incl AW045417: UI-M-BH1-akr-a-01-0-UI.s1 Mus musculus cDNA, 3 end/clone = UI-M-BH1-akr-a-01-0-UI/ | 990.4 | P | 581.5 | P | 828.4 | P | 1078.1 |
| | | clone_end = 3/gb = AW045417/gi = 5905946/ug = Mm 214/len = 336/STRA = rev | | | | | | | |
| 1 | 103451_at | Cluster Incl AI835159: UI-M-AQ0-aaf-e-10-0-UI S1 Mus musculus cDNA, 3 end/clone = UI-M-AQ0-aaf-e-10-0-UI/ | 268.9 | P | 231.9 | P | 284.7 | P | 287.6 |
| | | clone_end = 3/gb = AI835159/gi = 5469372/ug = Mm 21613/len = 139/STRA = for | | | | | | | |
| 1 | 103751_at | Cluster Incl AA833096 uc95g05.r1 Mus musculus cDNA, 5 end/clone = IMAGE-1433432/clone_end = 5/gb = AA833096/ | 1263.7 | P | 1267.9 | P | 1402.3 | P | 1350.4 |
| | | gi = 2906824/ug = Mm 24508/len = 289/STRA = for | | | | | | | |
| 1 | 103790_at | Cluster Incl AI846632: UI-M-AQ1-aef-h-03-0-UI.s1 Mus musculus cDNA, 3 end/clone = UI-M-AQ1-aef-h-03-0-UI/ | 1046.1 | P | 930.6 | P | 995.3 | P | 942.4 |
| | | clone_end = 3/gb = AI846632/gi = 5490538/ug = Mm.24950/len = 322/STRA = for | | | | | | | |
| 1 | 103971_at | Cluster Incl X84896: M. musculus mRNA for ATP receptor/cds = (164,1363)/gb = X84896/gi = 1166454/ug = Mm.25722/ | 451. 3 | P | 492.9 | P | 387.3 | P | 443.6 |
| | | len = 1760/STRA = for | | | | | | | |
| 1 | 104467_at | Cluster Incl AA763004: vw60b02 r1 Mus musculus cDNA, 5 end/clone = IMAGE-1248171/clone_end = 5/gb = AA763004/ | 689.3 | P | 573.4 | P | 702.1 | P | 726.3 |
| | | gl = 2812751/ug = Mm 2975/len = 545/STRA = for | | | | | | | |
| 1 | 104492_at | Cluster Incl U92702 Early B-cell factor 3/cds = (73,1863)/gb = U92702/gi = 2114427/ug = Mm 30282/len = 3015/STRA = rev | 320.2 | P | 335.9 | P | 288.5 | P | 269.1 |
| 1 | 92193_r_at | Cluster Incl D12619 Proprotein convertase subtilisin/kexin type 5/cds = (69,2816)/gb = D12619/gi = 220565/ug = Mm.3401/ | 346.4 | P | 278.6 | P | 271 | P | 279.1 |
| | | len = 2994/STRA = for | | | | | | | |
| 1 | 92211_at | Cluster Incl AF013969 Mus musculus antigen containing epitope to monoclonal antibody MMS-85/12 mRNA, partial cds/ | 584.7 | P | 539.8 | P | 767.7 | P | 397.1 |
| | | cds = (0,4704)/gb = AF013969/gi = 2384710/ug = Mm.34320/len = 4957/STRA = for | | | | | | | |
| 1 | 92225_f_at | Cluster Incl U58280: Mus musculus second largest subunit of RNA polymerase I (RPA2) mRNA, complete cds/ | 536.9 | P | 556.9 | P | 550.6 | P | 496.8 |
| | | cds = (55,3462)/gb = U58280/gi = 1621593/ug = Mm 34589/len = 3978/STRA = for | | | | | | | |
| 1 | 92294_at | Cluster Incl AW060793 UI-M-BH1-anu-g-02-0-UI s1 Mus musculus cDNA, 3 end/clone = UI-M-BH1-anu-g-02-0-UI/ | 372 | P | 283 | P | 319.6 | P | 299 |
| | | clone_end = 3/gb = AW060793/gi = 6008544/ug = Mm 3727/len = 453/STRA = rev | | | | | | | |
| 1 | 92908_at | Cluster Incl L36829 Mus musculus alphaA-crystallin-binding protein 1 (alphaA-CRYBP1) gene/cds = (103,8169)/gb = L36829/ | 102.9 | P | 103.4 | P | 154 | P | 67.2 |
| | | gi = 818007/ug = Mm 4808/len = 8601/STRA = rev | | | | | | | |
| 1 | 93144_at | Cluster Incl AI854602: UI-M-BH0-ake-c-05-0-UI s1 Mus musculus cDNA, 3 end/clone = UI-M-BH0-ake-c-05-0-UI/ | 813.5 | P | 609.3 | P | 626.7 | P | 639.2 |
| | | clone_end = 3/gb = AI854602/gi = 5498508/ug = Mm 5197/len = 481/STRA = for | | | | | | | |
| 1 | 93693_at | Cluster Incl AI835373: UI-M-AQ0-aah-h-10-0-UI s1 Mus musculus cDNA, 3 end/clone = UI-M-AQ0-aah-h-10-0-UI/ | 289.3 | P | 483.8 | P | 440.5 | P | 417.3 |
| | | clone_end = 3/gb = AI835373/gi = 5469586/ug = Mm 844/len = 285/STRA = for | | | | | | | |
| 1 | 94099_at | Cluster Incl AF049124 Mus musculus neuronal pentraxin 2 (Nptx2) mRNA, complete cds/cds = (67,1356)/gb = AF049124/ | 247.8 | P | 132.4 | P | 131.9 | P | 181.9 |
| | | gi = 2935463/ug = Mm 10099/len = 1608/STRA = for | | | | | | | |
| 1 | 94125_at | Cluster Incl AF030454 Epithelial V-like antigen/cds = (174,821)/gb = AF030454/gi = 3169827/ug = Mm 10715/len = 1279/ | 310.5 | P | 300.1 | P | 293.7 | P | 311.3 |
| | | STRA = for | | | | | | | |
| 1 | 94132_at | Cluster Incl X03920 M musculus GSHPx gene/cds = (37,642)/gb = X03920/gi = 51123/ug = Mm 1090/len = 923/STRA = for | 1257.2 | P | 1309.4 | P | 1240.3 | P | 1437.2 |
| 1 | 94706_s_at | Cluster Incl AI327488 mb01a01 yt Mus musculus cDNA, 5 end/clone = IMAGE-318888/clone_end = 5/gb = AI327488/ | 692.7 | P | 575.4 | P | 632.6 | P | 661.9 |
| | | gi = 4061917/ug = Mm 13854/len = 420/STRA = for | | | | | | | |
| 1 | 95326_at | Cluster Incl AF042180.Testis-specific protein, Y-encoded-like/cds = (55,1194)/gb = AF042180/gi = 3688779/ug = Mm 20445/ | 487.8 | P | 473.2 | P | 453.8 | P | 393.8 |
| | | len = 1455/STRA = for | | | | | | | |
| 1 | 95351_at | Clusler Incl AA414339 vc66b04.s1 Mus musculus cDNA, 5 end/clone = IMAGE-779503/clone_end = 5/gb = AA414339/ | 94.3 | P | 97.3 | P | 56.4 | P | 72 |
| | | gi = 2074486/ug = Mm.21121/len = 411/STRA = for | | | | | | | |
| 1 | 95400_l_at | Cluster Incl AI447783 mq84c02x1 Mus musculus cDNA, 3 end/clone = IMAGE-585410/clone_end = 3/gb = AI447783/ | 442.8 | P | 414.7 | P | 428.9 | P | 496 |
| | | gi = 4306405/ug = Mm 23936/len = 347/3TRA = rev | | | | | | | |
| 1 | 95992_at | Cluster Incl C79950 C79950 Mus musculus cDNA, 3 end/clone = J0074C04/clone_end = 3/gb = C79950/gi = 2520280/ | 897.7 | P | 1134.4 | P | 1010.6 | P | 940.1 |
| | | ug = Mm 25056/len = 530/STRA = rev | | | | | | | |
| 1 | 96546_r_at | Cluster Incl AI447510 mp69a04x1 Mus musculus cDNA, 3 end/clone = IMAGE-574446/clone_end = 3/gb = AI447510/ | 1796.3 | P | 2042.8 | P | 1995.3 | P | 1909.3 |
| | | gi = 4304492/ug = Mm 28/len = 291/STRA = for | | | | | | | |
| 1 | 96569_at | Cluster Incl AA268298 mz63a12 r1 Mus musculus cDNA, 5 end/clone = IMAGE-718078/clone_end = 5/gb = AA266298/ | 903.7 | P | 747.5 | P | 725.6 | P | 673 |
| | | gi = 1902420/ug = Mm 2972/len = 172/STRA = for | | | | | | | |
| 1 | 97083_at | Cluster Incl AA600468.vm78h03 r1 Mus musculus cDNA, 5 end/clone = IMAGE-1004405/clone_end = 5/gb = AA600468/ | 395.2 | P | 450.5 | P | 328.3 | P | 422.9 |
| | | gi = 2434093/ug = Mm 31138/len = 283/STRA = for | | | | | | | |
| 1 | 97163_g_at | Cluster Incl AI563699 vj83d06 y1 Mus musculus cDNA, 5 end/clone = lMAGE-935627/clone_end = 5/gb = AI563699/ | 742.1 | P | 652.3 | P | 603.8 | P | 524.1 |
| | | gi = 4522156/ug = Mm 35500/len = 392/STRA = rev | | | | | | | |
| 1 | 97176_at | Cluster Incl AI645547: vt97b07.x1 Mus musculus cDNA, 3 end/clone = IMAGE-1179061/clone_end = 3/gb = AI645547/ | 961.9 | P | 939.4 | P | 727 | P | 717.8 |
| | | gi = 4724022/ug = Mm 35777/len = 493/STRA = rev | | | | | | | |
| 1 | 97179_at | Clusler Incl C76543 C76543 Mus musculus cDNA, 3 end/clone = J0013B08/clone_end = 3/gb = C76543/gi = 2516873/ | 348.1 | P | 300 | P | 282 | P | 312.4 |
| | | ug = Mm 35801/len = 448/STRA = rev | | | | | | | |
| 1 | 97723_at | Cluster Incl X62537 Chromobox homolog 2 (Drosophila Pc class)/cds = (147,1706)/gb = X62537/gi = 52976/ug = Mm 3908/ | 429.8 | P | 398.4 | P | 391.7 | P | 345 |
| | | len = 1872/STRA = for | | | | | | | |
| 1 | 98344_f_at | Cluster Incl AI850438 UI-M-BG1-aih-a-04-0-UI s1 Mus musculus cDNA, 3 end/clone = UI-M-BG1-aih-a-04-0-UI/ | 1548.6 | P | 1394.6 | P | 1343.6 | P | 1343.4 |
| | | clone_end = 3/gb = AI850438/gi = 5494344/ug = Mm 43257/len = 304/STRA = rev | | | | | | | |
| 1 | 98778_at | Cluster Incl AI837543.UI-M-AL0-abs-d-04-0-UI s1 Mus musculus cDNA, 3 end/clone = UI-M-AL0-abs-d-04-0-UI/ | 260.6 | P | 217 | P | 255 | P | 233.8 |
| | | clone_end = 3/gb = AI837543/gi = 5471756/ug = Mm 4681/len = 246/STRA = for | | | | | | | |
| 1 | 99389_at | Cluster Incl M21673: Mouse productively rearranged T-cell receptor-beta (LH8-1) gene (V5 2-D2.1-J2 5 region), complete | 249.4 | P | 271.9 | P | 182.5 | P | 226.5 |
| | | cds/cds = (0,347)/gb = M21673/gi = 201830/ug = Mm.56958/len = 348/STRA = for | | | | | | | |
| 1 | 99847_at | Cluster Incl X73523 M musculus mRNA for Gal beta1, 3GalNAc alpha2,3-sialyltransferase/cds = (6,1019)/gb = X73523/ | 295.8 | P | 343.9 | P | 344.5 | P | 297.8 |
| | | gi = 402214/ug = Mm.6284/len = 1405/STRA = for | | | | | | | |
| 1 | 100344_at | Cluster Incl AI464141 vc34b10 x1 Mus musculus cDNA, 3 end/clone = IMAGE-776443/clone_end = 3/gb = AI464141/ | 1193.4 | P | 1118.2 | P | 1024.1 | P | 683.6 |
| | | gi = 4318171/ug = Mm 88145/len = 475/STRA = rev | | | | | | | |
| 1 | 100387_g_at | Cluster Incl AB019600 Mus musculus mRNA for caspase9, complete cds/cds = (0,1364)/gb = AB019600/gi = 6440941/ | 399.7 | P | 515.1 | P | 410.4 | P | 462.4 |
| | | ug = Mm 88829/len = 1365/STRA = for | | | | | | | |
| 1 | 101344_at | Cluster Incl AF019371 Cholecystokinin B receptor/cds = (0,1361)/gb = AF019371/gi = 2460176/ug = Mm.57011/len = 1362/ | 843.2 | P | 837.2 | P | 780.9 | P | 801.4 |
| | | STRA = for | | | | | | | |
| 1 | 96464_at | Cluster Incl N28179 MDB1515 Mus musculus cDNA, 3 end/clone_end = 3/gb = N28179/gi = 1146035/ug = Mm.556/len = 284/ | 1134.2 | P | 857 | P | 842.8 | P | 794.6 |
| | | STRA = rev | | | | | | | |
| 1 | 97004_at | Cluster Incl AJ133429.Mus musculus or17 gene/cds = (0,938)/gb = AJ133429/gi = 5869924/ug = Mm 88846/len = 939/ | 1589 | P | 1206.9 | P | 1207.5 | P | 1279.1 |
| | | STRA = for | | | | | | | |
| 1 | 97655_at | Cluster Incl Y11169 Desmocollin 3/cds = UNKNOWN/gb = Y11169/gi = 2765094/ug = Mm 89935/len = 2903/STRA = for | 267.3 | P | 96.9 | P | 171.5 | P | 191 |
| 1 | 97666_r_at | Cluster Incl C76213: C76213 Mus musculus cDNA, 3 end/clone = J0006A04/clone_end = 3/gb = C76213/gi = 2516543/ | 135 | P | 152.2 | P | 200.4 | P | 197.1 |
| | | ug = Mm.89955/len = 605/STRA = rev | | | | | | | |
| 1 | 100717_at | Cluster Incl U90926: Mus musculus putative TNF-resistance related protein mRNA, complete cds/cds = (136,492)/ | 526.9 | P | 402.5 | P | 486.1 | P | 633.2 |
| | | gb = U90926/gi = 1906634/ug = Mm 2548/len = 540/STRA = for | | | | | | | |
| 1 | 101226_at | Cluster Incl C77776 C77776 Mus musculus cDNA, 3 end/clone = J0037D08/clone_end = 3/gb = C77776/gi = 2518106/ | 864.6 | P | 713.8 | P | 677.8 | P | 821.8 |
| | | ug = Mm 5650/len = 622/STRA = rev | | | | | | | |
| 1 | 102478_f_at | Cluster Incl AW122990 UI-M-BH2 1-apb-b-09-0-UI.s1 Mus musculus cDNA, 3 end/clone = UI-M-BH2 1-apb-b-09-0-UI/ | 381.7 | P | 371.2 | P | 393.1 | P | 350.4 |
| | | clone_end = 3/gb = AW122990/gi = 6098520/ug = Mm 48699/len = 387/STRA = rev | | | | | | | |
| 1 | 94279_at | Cluster Incl AW125279 UI-M-BH2 1-apy-a-05-0-UI s1 Mus musculus cDNA, 3 end/clone = UI-M-BH2 1-apy-a-05-0-UI/ | 167.2 | P | 225.5 | P | 138.7 | A | 132.4 |
| | | clone_end = 3/gb = AW125279/gi = 6100809/ug = Mm 22514/len = 303/STRA = for | | | | | | | |
| 1 | 94886_at | Cluster Incl L18888 Calnexin/cds = (0,1775)/gb = L18888/gi = 309131/ug = Mm 23929/len = 1776/STRA = rev | 507.8 | P | 630 | P | 431.3 | P | 569.7 |
| 1 | 95486_at | Cluster Incl AA794134.vr42h04 s1 Mus musculus cDNA, 5 end/clone = IMAGE-1123351/clone_end = 5/gb = AA794134/ | 502.1 | P | 438 | P | 438.8 | P | 416.1 |
| | | gi = 2857089/ug = Mm 24997/len = 155/STRA = for | | | | | | | |
| 1 | 96023_at | Cluster Incl AI852472 UI-M-BHO-aip-f-10-0-UI s1 Mus musculus cDNA, 3 end/clone = UI-M-BH0-aip-l-10-0-UI/ | 166 | P | 155.5 | P | 111.9 | P | 173.4 |
| | | clone_end = 3/gb = AI852472/gi = 5496376/ug = Mm 25727/len = 402/STRA = rev | | | | | | | |
| 1 | 96032_at | Cluster Incl L19737.ATP synthase, H + transporting, mitochondrial F0 complex, subunit c (subunit 9), isoform 1/ | 130.5 | P | 129.9 | P | 126.9 | P | 119.4 |
| | | cds = (3,413)/gb = L19737/gi = 309287/ug = Mm 258/len = 499/STRA = for | | | | | | | |
| 1 | 96719_l_at | Cluster Incl X59382 Parvalbumin/cds = (56,388)/gb = X59382/gi = 509138/ug = Mm 2766/len = 615/STRA = for | 133.0 | P | 1320.6 | P | 1285.7 | P | 1202.9 |
| 1 | 97234_at | Cluster Incl AA792453: vs69d09 r1 Mus musculus cDNA, 5 end/clone = IMAGE-1151537/clone_end = 5/gb = AA792453/ | 226.4 | P | 217.3 | P | 189 | P | 198.5 |
| | | gi = 2855408/ug = Mm 27822/len = 351/STRA = for | | | | | | | |
| 1 | 97508_at | Cluster Incl M29462.Malate dehydrogenase, soluble/cds = (81,1085)/gb = M29462/gi = 199094/ug = Mm 3156/len = 1288/ | 493.8 | P | 570.4 | P | 555.7 | P | 401.7 |
| | | STRA = for | | | | | | | |
| 1 | 101981_at | Cluster Incl U09419 Mus musculus retinoid X receptor interacting protein (RIP15) mRNA, complete cds/cds = (130,1470)/ | 274.2 | P | 232.7 | P | 227.6 | P | 230.4 |
| | | gb = U09419/gi = 691713/ug = Mm. 968/len = 1841/STRA = for | | | | | | | |
| 1 | 102804_at | Cluster Incl X67279 Biliary glycoprotein 1/cds = UNKNOWN/gb = X67279/gi = 50170/ug = Mm 14114/len = 2922/STRA = for | 836.9 | P | 762.8 | P | 796.3 | P | 665.3 |
| 1 | 103013_at | Cluster Incl X77602 Upstream transcription factor 2/cds = (0,1028)/gb = X77602/gi = 609171/ug = Mm 15781/len = 1029/ | 553.2 | P | 606.5 | P | 367.9 | P | 552.4 |
| | | STRA = rev | | | | | | | |
| 1 | 103843_at | Cluster Incl M36777 Guanine nucleotide binding protein, alpha o/cds = (21,1085)/gb = M36777/gi = 193359/ug = Mm.20964/ | 316.1 | P | 290.8 | P | 280.2 | P | 267.9 |
| | | len = 1910/STRA = for | | | | | | | |
| 1 | 104629_at | Cluster Incl AI876546 uj60a09 x1 Mus musculus cDNA, 3 end/clone = IMAGE-1924312/clone_end = 3/gb = AI876546/ | 445.3 | P | 431.3 | P | 386.7 | P | 446.9 |
| | | gi = 5550595/ug = Mm 24331/len = 338/STRA = for | | | | | | | |
| 1 | 95575_r_at | Cluster Incl AI641995 ub74e05x1 Mus musculus cDNA, 3 end/clone = IMAGE-1383468/clone_end = 3/gb = AI641995/ | 251.4 | P | 264.6 | P | 250.2 | P | 232.6 |
| | | gi = 4720470/ug = Mm 27821/len = 385/STRA = for | | | | | | | |
| 1 | 95593_at | Cluster Incl AW125446 UI-M-BH2.3-aqh-h-05-0-UI s1 Mus musculus cDNA, 3 end/done = UI-M-BH2 3-aqh-h-05-0-UI/ | 796.6 | P | 1065.7 | P | 808.7 | P | 822.4 |
| | | clone_end = 3/gb = AW125446/gi = 6100976/ug = Mm 27902/len = 540/STRA = for | | | | | | | |
| 1 | 95610_at | Cluster Incl AA636547 vq76h11 s1 Mus musculus cDNA, 5 end/clone = IMAGE-1108293/clone_end = 5/gb = AA636547/ | 227 | P | 131.4 | P | 119.3 | P | 138.4 |
| | | gi = 2560326/ug = Mm 27989/len = 460/STRA = for | | | | | | | |
| 1 | 96126_at | Cluster Incl AF036894 Sphingosine phosphate lyase 1/cds = (60,1766)/gb = AF036894/gi = 2906010/ug = Mm.28119/ | 443.8 | P | 421 | P | 485.1 | P | 406.9 |
| | | len = 1911/STRA = for | | | | | | | |
| 1 | 96790_f_at | Cluster Incl AW125222: UI-M-BH2.1-aps-c-12-0-UI s1 Mus musculus cDNA, 3 end/clone = UI-M-BH2.1-aps-c-12-0-UI/ | 398.1 | P | 390.1 | P | 329.1 | P | 400.4 |
| | | clone_end = 3/gb = AW125222/gi = 6100752/ug = Mm 29098/len = 396/STRA = for | | | | | | | |
| 1 | 97437_f_at | Cluster Incl AW122483: UI-M-BH2.2-aou-f-01-0-UI.s1 Mus musculus cDNA, 3 end/clone = UI-M-BH2.2-aou-f-01-0-UI/ | 82.7 | P | 103.9 | P | 73.9 | P | 65.3 |
| | | clone_end = 3/gb = AW122483/gi = 6098005/ug = Mm30115/len = 277/STRA = for | | | | | | | |
| 1 | 99528_at | Cluster Incl AW122015.UI-M-BH2 3-aoj-h-12-0-UI s1 Mus musculus cDNA, 3 end/clone = UI-M-8H2 3-aoj-h-12-0-UI/ | 133.3 | A | 169.8 | P | 149.9 | P | 154.1 |
| | | clone_end = 3/gb = AW122015/gi = 6097421/ug = Mm 42151/len = 405/STRA = for | | | | | | | |
| 1 | 100941_at | Cluster Incl AF039567 Mus musculus Msx-interacting-zinc finger protein 1 (Miz1) mRNA, complete cds/cds = (26,1498)/ | 115.7 | P | 208.4 | P | 151.6 | P | 249.8 |
| | | gb = AF039567/gi = 2773147/ug = Mm 6370/len = 1847/STRA = for | | | | | | | |
| 1 | 102251_at | Cluster Incl AF047714 Mus musculus melastatin mRNA, complete cds/cds = (307,1935)/gb = AF047714/gi = 3047241/ | 682.1 | P | 633.2 | P | 479.9 | P | 472.5 |
| | | ug = Mm.10752/len = 2722/STRA = for | | | | | | | |
| 1 | 102681_at | Cluster Incl M24377 Early growth response 2/cds = (0,1262)/gb = M24377/gi = 198599/ug = Mm 1353/len = 1263/STRA = for | 812.1 | P | 778.2 | P | 538 | P | 721.7 |
| 1 | 103706_at | Cluster Incl AI036406 ub65e10 r1 Mus musculus cDNA, 5 end/clone = IMAGE-1382634/clone_end = 5/gb = AI036406/ | 260.6 | P | 259.1 | P | 303.1 | P | 261.7 |
| | | gi = 3260097/ug = Mm 24078/len = 380/STRA = for | | | | | | | |
| 1 | 104245_at | Cluster Incl AW124639 UI-M-BH2 1-apw-c-08-0-UI s1 Mus musculus cDNA, 3 end/clone = UI-M-BH2.1-apwc-08-0-UI/ | 1058.8 | P | 906.8 | P | 998.2 | P | 1071.9 |
| | | clone_end = 3/gb = AW124839/gi = 6100169/ug = Mm28288/len = 413/STRA = rev | | | | | | | |
| 1 | 104463_at | Cluster Incl AI157789 ue58d04 r1 Mus musculus cDNA, 5 end/clone = IMAGE-1495303/clone_end = 5/gb = AI157789/ | 356.8 | P | 203.2 | P | 299.8 | P | 372.3 |
| | | gi = 3686258/ug = Mm 29631/len = 443/STRA = for | | | | | | | |
| 1 | 104503_at | Cluster Incl AI563623 vx92d09.x1 Mus musculus cDNA, 3 end/clone = IMAGE-1282673/clone_end = 3/gb = AI563623/ | 201.3 | P | 128.7 | P | 106.1 | P | 123.9 |
| | | gi = 4522080/ug = Mm 30648/len = 482/STRA = for | | | | | | | |
| 1 | 92461_at | Cluster IncL AB021224-Mus musculus mRNA for membrane-type-4 matrix metalloproteinase, complete cds/cds = (85,1848)/ | 591.1 | P | 590.4 | P | 499.5 | P | 492.4 |
| | | gb = AB021224/gi = 5688930/ug = Mm 42047/len = 3490/STRA = for | | | | | | | |
| 1 | 93872_at | Cluster Incl AF014117 Mus musculus GDNF receptor alpha (GDNFR-alpha) mRNA, complete cds/cds = (913,2319)/ | 66.6 | P | 70.4 | P | 33.7 | P | 41.3 |
| | | gb = AF014117/gi = 2624960/ug = Mm.88367/len = 2549/STRA = for | | | | | | | |
| 1 | 94095_at | Cluster lncl AI552528 vx23a11.x1 Mus musculus cDNA, 3 end/clone = IMAGE-1265276/clone_end = 3/gb = AI552528/ | 243.6 | P | 262.5 | P | 248.9 | P | 243.1 |
| | | gi = 4484891/ug = Mm 100313/len = 496/STRA = rev | | | | | | | |
| 1 | 95891_at | Cluster lncl AI591977.mt35c08 y1 Mus musculus cDNA, 5 end/clone = IMAGE-623054/clone_end = 5/gb = AI591977/ | 368.5 | P | 353.4 | P | 304.8 | A | 309.3 |
| | | gi = 4601025/ug = Mm 24058/len = 354/STRA = rev | | | | | | | |
| 1 | 97726_at | Cluster Incl L35029 Glutamate receptor, ionotropic, NMDA2C (epsilon 3)/cds = (112,3723)/gb = L35029/gi = 602752/ | 742.2 | P | 639.1 | P | 748 | P | 748.3 |
| | | ug = Mm 39090/len = 3999/STRA = for | | | | | | | |
| 1 | 98771_at | Cluster Incl L25890 Eph receptor B2/cds = (92,2983)/gb = L25890/gi = 414593/ug = Mm.4652/len = 3104/STRA = for | 343.6 | P | 285.4 | P | 305 | P | 270.9 |
| 1 | 98853_at | Cluster Incl D30779: Mouse mRNA for phospholipase A2 receptor, complete cds/cds = (131,4594)/gb = D30779/gi = 1375042/ | 179.4 | P | 113.8 | P | 192.1 | P | 117.4 |
| | | ug = Mm.5092/len = 5569/STRA = for | | | | | | | |
| 1 | 99415_at | Cluster Incl U43321: Frizzled homolog 8, (Drosophila)/cds = (0,2057)/gb = U43321/gi = 1151259/ug = Mm.57055/len = 2058/ | 140.5 | P | 246.3 | P | 140.7 | P | 142.8 |
| | | STRA = for | | | | | | | |
| 1 | 100295_at | Cluster Incl M34597 Mouse lg germline lambda-chain gene Vx-J2-C2-region, complete cds, clone 30X2/cds = (0,368)/ | 699 | P | 636.5 | A | 486.9 | P | 543.3 |
| | | gb = M34597/gi = 197929/ug = Mm.86392/len = 369/STRA = for | | | | | | | |
| 1 | 94623_at | Cluster Incl U82698: Mus musculus enameiln mRNA, complete cds/cds = (300,4124)/gb = U82698/gi = 2737960/ | 256 | P | 235.4 | P | 233 | P | 222.2 |
| | | ug = Mm 8014/len = 4170/STRA = for | | | | | | | |
| 1 | 95848_at | Cluster Incl C76063: C76063 Mus musculus cDNA, 3 end/clone = J0003A08/clone_end = 3/gb = C76063/gi = 2516393/ | 348.2 | P | 280.2 | P | 299.4 | P | 296.1 |
| | | ug = Mm 24888/len = 626/STRA = rev | | | | | | | |
| 1 | 96996_at | Cluster Incl AJ245706 Mus musculus mRNA for nicotinic acetylcholine receptor subunit alpha6 (NICA6 gene)/ | 266 | P | 226.3 | A | 226.7 | P | 204.9 |
| | | cds = (80,1564)/gb = AJ245706/gi = 5777608/ug = Mm 88637/len = 1595/STRA = for | | | | | | | |
| 1 | 101595_at | Cluster Incl C78787 C78787 Mus musculus cDNA, 3 end/clone = J0055A07/clone_end = 3/gb = C78787/gi = 2979447/ | 167.2 | P | 149.5 | P | 211.2 | P | 238 |
| | | ug = Mm 5751/len = 387/STRA = rev | | | | | | | |
| 1 | 96310_at | Cluster Incl L07508 Mouse Golli-mpb mRNA, complete cds (alternate transcript from clone BG21)/cds = (274,861)/ | 164.3 | P | 229.5 | P | 194.5 | P | 177.9 |
| | | gb = L07508/gi = 193586/ug = Mm 2992/len = 1407/STRA = for | | | | | | | |
| 1 | 103062_at | Cluster Incl AB004664.Mus musculus DNA for Rab33B/cds = (316,1005)/gb = AB001664/gi = 2516240/ug = Mm 1664/ | 231.1 | A | 228.3 | P | 240.7 | P | 229.4 |
| | | len = 3421/STRA = for | | | | | | | |
| 1 | 99497_at | Cluster Incl AI852332 UI-M-BHO-ajo-a-06-O-UI a1 Mus musculus cDNA, 3 end/clone = UI-M-BHO-ajo-a-06-0-UI/ | 674.9 | P | 593.5 | A | 576 | P | 610.8 |
| | | clone_end = 3/gb = AI852332/gi = 5496238/ug = Mm 41637/len = 501/STRA = for | | | | | | | |
| 1 | 99954_at | Cluster Incl AW046474 UI-M-BH1-akz-a-08-0-UI s1 Mus musculus cDNA, 3 end/clone = UI-M-BH1-akz-a-08-0-UI/ | 736.5 | A | 700.6 | P | 592.6 | A | 657.6 |
| | | clone_end = 3/gb = AW046474/gi = 5907003/ug = Mm 439/len = 499/STRA = for | | | | | | | |
| 1 | 100405_at | Cluster Incl X56683 Mouse mRNA coding for modifier 2 protein/cds = (0,521)/gb = X56683/gi = 53164/ug = Mm 46536/ | 228.5 | P | 237.3 | P | 146.2 | A | 174.6 |
| | | len = 522/STRA = rev | | | | | | | |
| 1 | 102231_at | Cluster Incl AB017614 Mus musculus mRNA for OASIS protein, complete cds/cds = (27,1550)/gb = AB017614/gi = 4519620/ | 1235.4 | P | 1089 | P | 1130.5 | P | 1131.9 |
| | | ug = Mm.10353/len = 1908/STRA = rev | | | | | | | |
| 1 | 102711_at | Cluster Incl U85055: Mus musculus rap1/rap2 interacting protein mRNA, complete cds/cds = (0,1643)/gb = U85055/ | 365.7 | P | 340.3 | M | 386.4 | P | 563.3 |
| | | gi = 1814395/ug = Mm 1426/len = 2208/STRA = rev | | | | | | | |
| 1 | 103275_at | Cluster Incl U13836 Mus musculus vacuolar adenosine triphosphatase subunit Ac116 mRNA, complete cds/cds = (18,2537)/ | 186.7 | P | 174.2 | P | 236.6 | P | 225.5 |
| | | gb = U13836/gi = 3955095/ug = Mm.20869/len = 2557/STRA for | | | | | | | |
| 1 | 103766_at | Cluster Incl X97817 Semaphorin F/cds = (27,3260)/gb = X97817/gi = 1419018/ug = Mm 24733/len = 3949/STRA = for | 21.3 | A | 31 | P | 25.3 | Y | 17.6 |
| 1 | 93206_g_at | Cluster Incl M14167 Mouse ADA gene encoding adenosine deaminase, promoter region/cds = (142,144)/gb = M14167/ | 211.5 | P | 242.9 | P | 216.4 | P | 240.5 |
| | | gi = 191688/ug = Mm.56978/len = 145/STRA = for | | | | | | | |
| 1 | 93211_at | Cluster Incl AF032115 Mus musculus cysteine string protein mRNA complete cds/cds = (0,596)/gb = AF032115/ | 505.1 | P | 413.9 | A | 504.5 | P | 392.9 |
| | | gi = 2642628/ug = Mm 57022/len = 597/STRA = for | | | | | | | |
| 1 | 95295_s_at | Cluster Incl X59398.FMS-like tyrosine kinase 3/cds = (81,3083)/gb = X59398/gi = 50978/ug = Mm 194/len = 3521/STRA = for | 442.1 | P | 471.7 | P | 294 | M | 325.5 |
| 1 | 96557_at | Cluster Incl AB010833 Patched homolog 2/cds = (0,3548)/gb = AB010833/gi = 2842557/ug = Mm 28529/len = 3549/STRA = for | 370.5 | P | 433.9 | P | 424.5 | A | 340.2 |
| 1 | 97097_at | Cluster Incl AW125669: UI-M-BH2 2-aqi-c-12-0-UI S1 Mus musculus cDNA, 3 end/clone = UI-M-BH2 2-aqi-c-12-0-UI/ | 236.9 | A | 282.5 | P | 231.8 | P | 217 |
| | | clone_end = 3/gb = AW125669/gi = 6101199/ug = Mm 3183/len = 507/STRA = for | | | | | | | |
| 1 | 97136_at | Cluster IncI AA571388 vl77a12 s1 Mus musculus cDNA, 3 end/clone = IMAGE-978238/clone_end = 3/gb = AA571388/ | 2.1 | P | 54.6 | P | −61.2 | A | 83.3 |
| | | gi = 2346317/ug = Mm 34930/len = 440/STRA = for | | | | | | | |
| 1 | 98349_at | Cluster Incl X62646 M musculus mRNA for IL-6 signal transducer (gp130)/cds = (88,2841)/gb = X62646/gi = 840816/ | 24.7 | P | 127.5 | P | 73.5 | P | 53.8 |
| | | ug = Mm 4364/len = 2995/STRA = for | | | | | | | |
| 1 | 100750_at | Cluster IncI AA414903 vc50b09.r1 Mus musculus cDNA, 3 end/clone = IMAGE-777977/clone_end = 3/gb = AA414903/ | 244 | P | 174.5 | P | 198.9 | P | 187.2 |
| | | gi = 2075133/ug = Mm 3808/len = 151/STRA = rev | | | | | | | |
| 1 | 101661_r_at | Cluster Incl AA420337 vc51dO3 r1 Mus musculus cDNA, 3 end/clone = IMAGE-778085/clone_end = 3/gb = AA420337/ | 43 | P | 65.5 | P | 26 | A | 49.6 |
| | | gi = 2080894/ug = Mm.6761/len = 281/STRA = rev | | | | | | | |
| 1 | 94072_g_at | Cluster Incl AI847904 UI-M-AI1-afl-a-09-0-UI.s1 Mus musculus cDNA, 3 end/clone = UI-M-AI1-afl-o-09-0-UI/clone_end = 3/ | 729 | P | 596.5 | P | 659 | P | 554.8 |
| | | gb = AI847904/gi = 5491810/ug = Mm 22114/len = 350/STRA = for | | | | | | | |
| 1 | 97222_at | Cluster Incl AI845921 UI-M-AK1-aex-d-11-0-UI s1 Mus musculus cDNA, 3 end/clone = UI-M-AK1-aea-d-11-0-UI/ | 621.8 | P | 617.6 | A | 540.6 | P | 522.2 |
| | | clone_end = 3/gb = AI845921/gi = 5489827/ug = Mm.27756/len = 298/STRA = for | | | | | | | |
| 1 | 97821_at | Cluster Incl AI646056 vw54c11.x1 Mus musculus cDNA, 3 end/clone = IMAGE-1247636/clone_end = 3/gb = AI646056/ | 569.8 | P | 498.2 | P | 500.4 | P | 537.8 |
| | | gi = 4724531/ug = Mm 28202/len = 561/STRA = rev | | | | | | | |
| 1 | 100619_r_at | Cluster Incl AA062013 mj83h01 r1 Mus musculus cDNA, 5 end/clone = IMAGE-482737/clone_end = 5/gb = AA062013/ | 442.1 | P | 344.6 | P | 317.4 | M | 335.5 |
| | | gi = 1555829/ug = Mm 658/len = 529/STRA = for | | | | | | | |
| 1 | 95545_at | Cluster Incl X04480.lnsulin-like growth factor 1/cds = (152,535)/gb = X04480/gi = 51801/ug = Mm 2770/len = 696/STRA = for | 594.1 | P | 562.1 | A | 547.5 | A | 515 |
| 1 | 98443_at | Cluster Incl AI882416 uc01c09 r1 Mus musculus cDNA, 5 end/clone = IMAGE-1396720/clone_end = 5/gb = AI882416/ | 568 | P | 664.6 | P | 506.6 | A | 596.1 |
| | | gi = 5567505/ug = Mm 34519/len = 338/STRA = for | | | | | | | |
| 1 | 99463_at | Cluster Incl X63023 Cytochrome P450, steroid Inducible 3a13/cds = (83,1594)/gb = X63023/gi = 50634/ug = Mm 4094/ | 105.5 | P | 96 | A | 135.2 | P | 104.2 |
| | | len = 1881/STRA = for | | | | | | | |
| 1 | 100020_at | Cluster Incl J04036 Solute carrier family 4 (anlon exchanger), member 2/cds = (182,3895)/gb = J04036/gi = 192132/ | 374.3 | P | 485.5 | P | 378.4 | A | 368.2 |
| | | ug = Mm 4580/len = 4088/STRA = for | | | | | | | |
| 1 | 101417_at | Cluster Incl AF020191: Mus musculus proline-rich protein 13 mRNA, partial cds/cds = (0,511)/gb = AF020191/gi = 2501952/ | 247.3 | M | 296.4 | P | 255.4 | P | 239.3 |
| | | ug = Mm.7412/len = 1370/STRA = for | | | | | | | |
| 1 | 101840_at | Cluster Incl L06864 Mus musculus epidermal growth factor receptor (EGFR) mRNA, partial cds/cds = (0,363)/gb = L06864/ | 171.3 | P | 137 | A | 128.2 | P | 185.5 |
| | | gi = 193001/ug = Mm 8534/len = 364/STRA = for | | | | | | | |
| 1 | 102242_at | Cluster Incl AF050182 Mus musculus PERIOD 3 (Per3) mRNA, complete cds/cds = (357,3698)/gb = AF050182/gi = 3136149/ | 648.7 | P | 387.1 | A | 396.8 | P | 559.3 |
| | | ug = Mm 10723/len = 4863/STRA = for | | | | | | | |
| 1 | 104238_at | Cluster Incl U60881: ADP-ribosyltransferase 5/cds = (382,1311)/gb = U60881/gi = 1522688/ug = Mm.2816/len = 1547/ | 291.1 | P | 274.3 | A | 345.8 | P | 331.8 |
| | | STRA = for | | | | | | | |
| 1 | 92745_at | Cluster Incl AB005458 Homeo box A9/cds = (446,763)/gb = AB005458/gi = 3080549/ug = Mm 4694/len = 764/STRA = for | 87.1 | P | 88.2 | P | 69.8 | P | 71.3 |
| 1 | 93689_at | Cluster Incl AW122071 UI-M-BH2 2-aoo-e-12-0-UI s1 Mus musculus cDNA, 3 end/clone = UI-M-BH2 2-aoo-e-12-0-UI/ | 661.2 | P | 603 | P | 567.5 | P | 571.4 |
| | | clone_end = 3/gb = AW122071/gi = 6097478/ug = Mm 840/len = 413/STRA = for | | | | | | | |
| 1 | 94135_at | Cluster Incl M76987 Transcription factor CP2/cds = UNKNOWN/gb = M76987/gi = 191802/ug = Mm 1139/len = 4023/ | 516.2 | P | 334.5 | P | 352.4 | P | 387 |
| | | STRA = for | | | | | | | |
| 1 | 94171_at | Cluster Incl AF051367 Mus musculus pactolus mRNA, complete cds/cds = (63,2279)/gb = AF051367/gi = 3287490/ | 249.8 | P | 294.5 | P | 263.9 | P | 249.9 |
| | | ug = Mm 12872/len = 2628/STRA = for | | | | | | | |
| 1 | 97096_at | Cluster Incl J02935 Mouse cAMP-dependent protein kinase type II regulatory subunit mRNA, 3 end/cds = (0,1205)/ | 538.3 | P | 509.9 | A | 390 | P | 485.2 |
| | | gb = J02935/gi = 200356/ug = Mm 3176/len = 1533/STRA = for | | | | | | | |
| 1 | 98867_at | Cluster Incl AI848793 UI-M-AJ1-ahg-d-12-0-UI s1 Mus musculus cDNA, 3 end/clone = UI-M-AJ1-ahg-d-12-0-UI/ | 167.5 | P | 185.8 | P | 115.8 | M | 140.2 |
| | | clone_end = 3/gb = AI848793/gi = 5492699/ug = Mm 5152/len = 388/STRA = rev | | | | | | | |
| 1 | 99810_at | Cluster Incl X91864 Glutathione peroxidase 2, pseudogene 1/cds = (0,158)/gb = X91864/gi = 1359980/ug = Mm 57225/ | 247.2 | P | 332.3 | A | 184 | P | 210.9 |
| | | len = 159/STRA = for | | | | | | | |
| 1 | 102557_at | Cluster Incl U62673 Mus musculus histone H2a(A)-613, histone H2a(B)-613, and histone H2b-613 (H2b) genes, complete | 110.5 | P | 91.2 | M | 102.5 | P | 89.5 |
| | | cds/cds = (0,392)/gb = U62673/gi = 1458138/ug = Mm 57213/len = 393/STRA = for | | | | | | | |
| 1 | 94551_at | Cluster Incl AI853061 UI-M-BH0-ajn-g-12-0-UI s1 Mus musculus cDNA, 3 end/clone = UI-M-BH0-ajn-g-12-0-UI/ | 268.9 | P | 237.3 | P | 239.7 | A | 268.5 |
| | | clone_end = 3/gb = AI853061/gi = 5496967/ug = Mm 29087/len = 363/STRA = for | | | | | | | |
| 1 | 94556_at | Cluster Incl AI746846 uI08b08.y1 Mus musculus cDNA, 5 end/clone = IMAGE-2076183/clone_end = 5/gb = AI746846/ | 95.3 | P | 122.4 | P | 115.3 | P | 122.8 |
| | | gi = 5125110/ug = Mm 29101/len = 596/STRA = for | | | | | | | |
| 1 | 102372_at | Cluster Incl M90766 Mouse Ig active joining chain (j chain) mRNA of the b allele, complete cds/cds = (21,500)/gb = M90766/ | 165 | P | 242.5 | P | 172.6 | P | 199.3 |
| | | gi = 194473/ug = Mm.1192/len = 501/STRA = for | | | | | | | |
| 1 | 103614_at | Cluster Incl AW047899 UI-M-BH1-ain-a-06-0-UI s1 Mus musculus cDNA, 3 end/clone = UI-M-BH1-aln-a-06-0-UI/ | 167.4 | A | 87.7 | A | 181.5 | P | 130.3 |
| | | clone_end = 3/gb = AW047B99/gi = 5908428/ug = Mm 20225/len = 403/STRA = rev | | | | | | | |
| 1 | 103840_at | Cluster Incl AJ01,1923 Mus musculus mRNA for cell cycle checkpoint protein/cds = (75,2138)/gb = AJ011923/gi = 3758877/ | 203.9 | P | 202.7 | P | 47 | A | 59.2 |
| | | ug = Mm 20947/len = 2640/STRA = for | | | | | | | |
| 1 | 104036_at | Cluster Incl AI845447 UI-M-A01-aej-f-01-0-UI s1 Mus musculus cDNA, 3 end/clone = UI-M-AO1-aej-f-01-0-UI/ | 211.6 | P | 108.9 | A | 214.6 | P | 113.3 |
| | | clone_end = 3/gb = AI845447/gi = 5489353/ug = Mm 21440/len = 343/STRA = for | | | | | | | |
| 1 | 96806_at | Cluster Incl AI843802 UI-M-AN1-afc-e-12-0-UI.s1 Mus musculus cDNA, 3 end/clone = UI-M-AN1-afc-e-12-0-UI/ | 385.2 | P | 397.5 | P | 356 | P | 327.7 |
| | | clone_end = 3/gb = AI843802/gi = 5478015/ug = Mm 29209/len = 537/STRA = for | | | | | | | |
| 1 | 98457_at | Cluster Incl AF020195 Mus musculus pancreas sodium bicarbonate cotransporter mRNA, complete cds/cds = (29,3268)/ | 149.4 | A | 81.9 | A | 93.3 | P | 104.4 |
| | | gb = AF020195/gi = 3298571/ug = Mm.34747/len = 3337/STRA = for | | | | | | | |
| 1 | 102265_at | Cluster Incl X59060 Myogenic factor 6/cds = (25,753)/gb = X59060/gi = 53297/ug = Mm 11/len = 1258/STRA = for | 275.6 | P | 305.7 | A | 182.4 | A | 270.2 |
| 1 | 102891_at | Cluster Incl D86527: Werner syndrome homolog (human)/cds = (183,4388)/gb = D86527/gi = 2130974/ug = Mm 15446/ | 264.2 | P | 225 | P | 226.7 | A | 273 |
| | | len = 4955/STRA = rev | | | | | | | |
| 1 | 104273_at | Cluster Incl U95215 Bile acid CoA-amino acid n-acyltransferase/cds = (189,1451)/gb = U95215/gi = 2130557/ug = Mm.2859/ | 296.5 | P | 200.8 | P | 179 | P | 199.1 |
| | | len = 1870/STRA = rev | | | | | | | |
| 1 | 92895_at | Cluster Incl AF038857 untitled/cds = (0,2297)/gb = AF038857/gi = 2952265/ug = Mm.4774/len = 2298/STRA = for | 175.6 | A | 115.2 | P | 165.1 | P | 155.4 |
| 1 | 93185_at | Cluster Incl AW045808 UI-M-BH1-akt-h-04-0-UI.s1 Mus musculus cDNA, 3 end/clone = UI-M-BH1-akt-h-04-0-UI/ | 187.1 | P | 252.6 | P | 220.3 | P | 124.4 |
| | | clone_end = 3/gb = AW0458O8/gi = 5906337/ug = Mm 5473/len = 367/STRA = rev | | | | | | | |
| 1 | 94762_at | Cluster Incl U92972 Mus musculus protease-activated receptor 3 (PAR3) mRNA, complete cds/cds = (152,1261)/ | 362.6 | P | 331.3 | P | 304.1 | P | 370.4 |
| | | gb = U92972/gi = 1938376/ug = Mm 1612/len = 2409/STRA = rev | | | | | | | |
| 1 | 102580_r_at | Cluster Incl M11988 Homeo box A6/cds = (0,293)/gb = M11988/gi = Mm.57244/len = 294/STRA = for | 301 | P | 271.8 | A | 311.6 | P | 275.2 |
| 1 | 101233_at | Cluster Incl C78616.C78616 Mus musculus cDNA, 3 end/clone = J0052A12/clone_end = 3/gb = C78616/gi = 2518946/ | 72 | P | 75.8 | P | 63.9 | P | 43 |
| | | ug = Mm 5725/len = 541/STRA = rev | | | | | | | |
| 1 | 101687_r_at | Cluster Incl C79518.C79518 Mus musculus cDNA,3 end/clone = J0067E04/clone_end = 3/gb = C79518/gi = 2519848/ | 91.4 | P | 43.3 | A | 68.8 | M | 58.4 |
| | | ug = Mm 36431/len = 252/STRA = rev | | | | | | | |
| 1 | AFFX-BioDn- | E. Coli BioD 3 prime | 116.7 | A | 189.7 | P | 138.4 | P | 138.5 |
| | 3_at | | | | | | | | |
| 1 | 93788_at | Cluster Incl M37831.Cytochrome C oxidase, subunit IV/cds = (1,510)/gb = M37831/gi = 459880/ug = Mm 2136/len = 610/ | 166.1 | P | 173.8 | A | 167.2 | P | 153.7 |
| | | STRA = for | | | | | | | |
| 1 | 94235_at | Cluster Incl AW050034 UI-M-BH1-amm-b-10-0-UI s1 Mus musculus cDNA, 3 end/clone = UI-M-BH1-amm-b-10-0-UI/ | 61.4 | A | 44.2 | P | 53.7 | A | 27.4 |
| | | clone_end = 3/gb = AW050034/gi = 5910563/ug = Mm.22327/len = 531/STRA = for | | | | | | | |
| 1 | 96044_at | Cluster Incl AF067660: Bcl2-like 10/cds = (142,717)/gb = AF067660/gi = 3955265/ug = Mm 25988/len = 1225/STRA = for | 326.5 | M | 362.6 | P | 338.1 | P | 203.8 |
| 1 | 98526_r_at | Cluster Incl AI854606 UI-M-BH0-ake-c-09-0-UI s1 Mus musculus cDNA, 3 end/clone = UI-M-BH0-ake-c-09-0-UI/ | 342 | P | 184 | M | 150.4 | A | 303.1 |
| | | clone_end = 3/gb = AI854606/gi = 5498512/ug = Mm 35829/len = 472/STRA = rev | | | | | | | |
| 1 | 102765_at | Cluster Incl AF071317-Mus musculus COP9 complex subunit 7b (COPS7b) mRNA, complete cds/cds = (130,924)/ | 471.9 | A | 191.6 | P | 359.3 | A | 457.8 |
| | | gb = AF071317/gi = 3309175/ug = Mm 12973/len = 1990/STRA = for | | | | | | | |
| 1 | 102771_at | Cluster Incl AF091628 Mus musculus ERG-associated protein ESET mRNA, complete cds/cds = (101,4024)/gb = AF091628/ | 294.4 | P | 403.4 | A | 258.6 | P | 370.3 |
| | | gi = 3644041/ug = Mm 13162/len = 4602/STRA = rev | | | | | | | |
| 1 | 102861_at | Cluster Incl AF028739 Imprinted polyspecific membrane transporter-like gene 1/cds = (179,1399)/gb = AF02B739/ | 259 | A | 188.5 | M | 255.8 | P | 236.1 |
| | | gi-2583224/ug = Mm 15093/len = 1466/STRA = for | | | | | | | |
| 1 | 96184_at | Cluster Incl AIB35242.UI-M-AQ0-aaa-h-10-0-UI s1 Mus musculus cDNA, 3 end/clone = UI-M-AQ0-aaa-h-10-0-UI/ | 658.5 | P | 769.5 | P | 802.1 | P | 782 |
| | | clone_end = 3/gb = AI835242/gi = 5489455/ug = Mm 28462/len = 294/STRA = for | | | | | | | |
| 1 | 98970_at | Cluster Incl AB016044 Mus musculus mRNA for GGPP synthase, complete cds/cds = (107,1009)/gb = AB016044/ | 154.6 | P | 127.9 | P | 100.5 | A | 182 |
| | | gi = 4586887/ug = Mm 36520/len = 1025/STRA = for | | | | | | | |
| 1 | 100961_at | Cluster Incl AF012871.Long(electocardiographic) QT syndrome 2/cds = (321,3809)/gb = AF012871/gi = 2582014/ | 276 | M | 429.7 | P | 353.8 | A | 383.7 |
| | | ug = Mm 6539/len = 4221/STRA = for | | | | | | | |
| 1 | 101399_at | Cluster Incl M77174 Perlecan (heparan sulfate proteoglycan 2)/cds = (578,11701)/gb = M77174/gi = 200295/ug = Mm 7257/ | 546.7 | M | 585 | M | 369.2 | A | 471.6 |
| | | len = 12667/STRA = for | | | | | | | |
| 1 | 101942_at | Cluster Incl AA839050: vw47a01.r1 Mus musculus cDNA, 5 end/clone = IMAGE-1246920/clone_end = 5/gb = AA839050/ | 313.9 | P | 270.2 | M | 472.1 | P | 519.8 |
| | | gi = 2915145/ug = Mm.854/len = 511/STRA = for | | | | | | | |
| 1 | 104239_at | Cluster Incl AW208513: uo60102.x1 Mus musculus cDNA, 3 end/clone = IMAGE-2646939/clone_end = 3/gb = AW208513/ | 105.6 | P | 60.1 | P | 81.6 | P | 54.4 |
| | | gi = 6514453/ug = Mm 28172/len = 461/STRA = for | | | | | | | |
| 1 | 104500_at | Cluster Incl L34111: Alpha-L-iduronidase/cds = (26,1930)/gb = L34111/gi = 623169/ug = Mm 3054/len = 3109/STRA = for | 164.1 | A | 141.1 | P | 122.9 | P | 146 |
| 1 | 92271_at | Cluster Incl X63963 Paired box gene 6/cds = (162,1472)/gb = X63963/gi = 460050/ug = Mm 3608/len = 2481/STRA = for | 547.5 | M | 540 | A | 521.7 | A | 475.2 |
| 1 | 92458_at | Cluster Incl AJ003133 Mus musculus mRNA for ORC1-related protein, complete cds/cds = (123,2645)/gb = AJ003133/ | 237.3 | P | 195.5 | P | 184 | A | 284.1 |
| | | gi = 4034784/ug = Mm 42024/len = 3017/STRA = for | | | | | | | |
| 1 | 94177_at | Cluster Incl Y15733 Mus musculus mRNA for 17-beta-hydroxysteroid dehydrogenase type 7/cds = (03,1067)/gb = Y15733 | 67.8 | A | 84.8 | P | 55.3 | P | 34.5 |
| | | gi = 3319970/ug = Mm 12882/len = 1756/STRA = for | | | | | | | |
| 1 | 96593_at | Cluster Incl X87257: ELK1, member of ETS oncogene family/cds = (473,1762)/gb = X87257/gi = 836634/ug = Mm 3064/ | 103.9 | P | 87.6 | M | 130.8 | P | 109.4 |
| | | len = 2286/STRA = for | | | | | | | |
| 1 | 102078_at | Cluster Incl AF066075 untitled/cds = (127,474)/gb = AF066075/gi = 4092929/ug = Mm 42082/len = 714/STRA = for | 41.4 | P | 43.1 | A | 30 | A | 42.7 |
| 1 | 94541_at | Cluster Incl AI839549 UI-M-AN0-acn-h-08-0-UI.s1 Mus musculus cDNA, 3 end/clone = UI-M-AN0-acn-h-08-0-UI/ | 149.7 | A | 205 | P | 184.1 | A | 207.9 |
| | | clone_end = 3/gb = AI839549/gi = 5473762/ug = Mm 29065/len = 162/STRA = for | | | | | | | |
| 1 | 101513_at | Cluster Incl AI840454 UI-M-AN0-aci-c-01-o-UI s1 Mus musculus cDNA, 3 end/clone = UI-M-AN0-aci-c-01-o-UI/ | 162.1 | M | 139.6 | A | 160.7 | M | 133 .4 |
| | | clone_end = 3/gb = AI840454/gi = 5474667/ug = Mm.836/len = 446/STRA = for | | | | | | | |
| 1 | 102995_s_at | Cluster Incl M13226 Granzyme A/cds = (198,971)/gb = M13226/gi = 201048/ug = Mm.15510/len = 1039/STRA = for | 266 | M | 269.7 | P | 251.1 | A | 215.3 |
| 1 | 103361_at | Cluster Incl AI853856 UI-M-BH0-ajr-e-10-0-UI.s1 Mus musculus cDNA, 3 end/clone = UI-M-BH0-ajr-e-10-0-UI/ | 51.9 | A | 33.6 | A | 47. 8 | P | 19.9 |
| | | clone_end = 3/gb = AI853856/gi = 5497762/ug = Mm 18506/len = 374/STRA = rev | | | | | | | |
| 1 | 104581_at | Cluster Incl AI845438 UI-M-AO1-aej-d-10-0-UI s1 Mus musculus cDNA, 3 end/clone = UI-M-AO1-aoj-d-10-0-UI/ | 32.5 | M | 69.7 | P | 45.6 | M | 48 |
| | | clone_end = 3/gb = AI845438/gi = 5489339/ug = Mm 23975/len = 369/STRA = for | | | | | | | |
| 1 | 104708_at | Cluster Incl AI463490 vw62d08.x1 Mus musculus cDNA, 3 end/clone = IMAGE-1248399/clone_end = 3/gb = AI463490/ | 240.7 | P | 376.2 | P | 213 | A | 277 |
| | | gi = 4317520/ug = Mm 24408/len = 180/STRA = for | | | | | | | |
| 1 | 102649_s_at | Cluster Incl D64162 Mouse clone Rae-123 mRNA for cell surface protein, complete cds/cds = (92,853)/gb = D84162/ | 180.9 | A | 151.3 | A | 173.9 | P | 164.7 |
| | | gi = 1311547/ug = Mm 1326/len = 1242/STRA = for | | | | | | | |
| 1 | 104449_at | Cluster Incl X81202: Glycine receptor, beta subunit/cds = (0,1455)/gb = X81202/gi = 557654/ug = Mm 29422/len = 1465/ | 147.6 | A | 148.1 | A | 137.7 | A | 129.5 |
| | | STRA = for | | | | | | | |
| 1 | 104509_at | Cluster Incl AF059213 Mus musculus cholesterol 25-hydroxylase mRNA, complete cds/cds = (9,905)/gb = AF059213/ | 199 | P | 161.5 | A | 145.1 | P | 166.2 |
| | | gi = 4038305/ug = Mm 30824/len = 1365/STRA = rev | | | | | | | |
| 1 | 92312_at | Cluster Incl U55772 Mus musculus p170 phosphatidylinositol 3-kinase mRNA, complete cds/cds = (57,4586)/gb = U55772/ | 177.4 | P | 131.8 | A | 181.5 | P | 150.4 |
| | | gi = 1305537/ug = Mm 3810/len = 5067/STRA = for | | | | | | | |
| 1 | 92493_at | Cluster Incl Y10926 M musculus mRNA for transcription factor RBP-L/cds = (103,1650)/gb = Y10926/gi = 2052118/ | 208 | P | 200.7 | P | 190.7 | A | 150.5 |
| | | ug = Mm 42177/len = 2456/STRA = for | | | | | | | |
| 1 | 93452_at | Cluster Incl AF031896 Cerberus 1/cds = (82,900)/gb = AF031896/gi = 2654026/ug = Mm 6780/len = 1752/STRA = for | 186 | A | 118.3 | P | 173.5 | A | 180 |
| 1 | 97143_at | Cluster Incl C79450 C79450 Mus musculus cDNA, 3 end/clone = J0066E08/clone_end = 3/gb = C79450/gi = 2519780/ | 91.7 | P | 193.3 | P | 232.8 | P | 161.5 |
| | | ug = Mm 34943/len = 579/STRA = rev | | | | | | | |
| 1 | 97156_at | Cluster Incl D32200 Mouse gene for Hes-3/cds = (0,527)/gb = D32200/gi = 699611/ug = Mm 3516/len = 528/STRA = for | 293.1 | P | 257.9 | A | 108 | A | 288.2 |
| 1 | 97701_at | Cluster Incl AI181906 ud73e11 x1 Mus musculus cDNA, 3 end/clone = IMAGE-1451564/clone_end = 3/gb = AI181906/ | 159.1 | P | 146.1 | A | 154.1 | A | 158.2 |
| | | gi = 3732544/ug = Mm 38004/len = 387/STRA = rev | | | | | | | |
| 1 | 99893_at | Cluster Incl AF020737: Fibroblast growth factor 13/cds = (9,743)/gb = AF020737/gi = 2444476/ug = Mm 7995/len = 817/ | 87.8 | P | 68.3 | A | 68.1 | P | 85.5 |
| | | STRA = for | | | | | | | |
| 1 | 102621_at | Cluster Incl AF090866 Mus musculus CDO (Cdo) mRNA, complete cds/cds = (29,3781)/gb = AF090866/gi = 3644025/ | 296.2 | A | 285.4 | P | 323.2 | P | 265.3 |
| | | ug = Mm 20439/len = 3914/STRA = for | | | | | | | |
| 1 | 97665_1_at | Cluster Incl C76213 C76213 Mus musculus cDNA, 3 end/clone = J0006A04/clone_end = 3/gb = C76213/gi = 2516543/ | 126.7 | A | 103.3 | A | −50.7 | P | 152.4 |
| | | ug = Mm 89955/len = 605/STRA = rev | | | | | | | |
| 1 | 101614_at | Cluster Incl AA672848 vp57b02 r1 Mus musculus cDNA, 3 end/clone = IMAGE-1080747/clone_end = 3/gb = AA672648/ | 169.6 | A | 102.3 | A | 131.4 | A | 161.5 |
| | | gi = 2644865/ug = Mm 6126/len = 404/STRA = rev | | | | | | | |
| 1 | 102143_at | Cluster Incl AW047018: UI-M-BH1-alp-h-06-0-UI s1 Mus musculus cDNA, 3 end/clone = UI-M-BH1-alp-h-06-0-UI/ | 399.4 | A | 238.8 | P | 242.3 | A | 368.6 |
| | | clone_end = 3/gb = AW047018/gi = 5907547/ug = Mm 44276/len = 396/STRA = rev | | | | | | | |
| 2 | 92573_at | Cluster Incl AB021743 Mus musculus mRNA for PR65 (type 2A serine/threonine protein phosphatase A subunit), complete | 1832.6 | P | 2116.1 | P | 2307.6 | P | 2012.9 |
| | | cds/cds = (0,1769)/gb = AB021743/gi = 4514638/ug = Mm. 1034/len = 1770/STRA = for | | | | | | | |
| 2 | 92599_at | Cluster Incl AF029843 Mus musculus phosphoglycerate mutase muscle-specific subunit mRNA, complete cds/ | 551.3 | P | 846.6 | P | 1527.2 | P | 861 |
| | | cds = (45,806)/gb = AF029843/gi = 3043914/ug = Mm 10738/len = 836/STRA = for | | | | | | | |
| 2 | 92621_at | Cluster Incl X97982 M musculus mRNA for poly(C)-binding protein, splice variant E/cds = (221,1270)/gb = X97982/ | 2330.5 | P | 2402.2 | P | 3110.6 | P | 2454.4 |
| | | gi = 1360002/ug = Mm.111/len = 1655/STRA = rev | | | | | | | |
| 2 | 92637_at | Cluster Incl J03928 Phosphofructokinase, liver, B-type/cds = (42,2384)/gb = J03928/gi = 200297/ug = Mm 1166/len = 2688/ | 575.8 | P | 868.9 | P | 725.8 | P | 799.3 |
| | | STRA = for | | | | | | | |
| 2 | 92775_at | Cluster Incl AA656757: vr50c03.s1 Mus musculus cDNA, 5 end/clone = IMAGE-1124068/clone_end = 5/gb = AA656757/ | 514.9 | P | 583.4 | P | 740 | P | 634.3 |
| | | gi = 2592911/ug = Mm 12267/len = 407/STRA = for | | | | | | | |
| 2 | 92777_at | Cluster Incl M32490 Insulin-like growth factor binding protein 10/cds = (189,1328)/gb = M32490/gi = 192909/ug = Mm 1231/ | 112.1 | P | 136 | P | 417.6 | P | 195.3 |
| | | len = 2018/STRA = for | | | | | | | |
| 2 | 92787_at | Cluster Incl AI845902: UI-M-AK1-aex-b-07-0-UI s1 Mus musculus cDNA, 3 end/clone = UI-M-AK1-aex-b-07-0-UI/ | 291.4 | P | 725.3 | P | 746.2 | P | 387 |
| | | clone_end = 3/gb = AI845902/gi = 5489808/ug = Mm.12459/len = 358/STRA = for | | | | | | | |
| 2 | 93104_at | Cluster Incl Z16410 M musculus btg1 mRNA/cds = (0,515)/gb = Z16410/gi = 50187/ug = Mm 16596/len = 516/STRA = for | 1471 | P | 1358.7 | P | 2184. 6 | P | 1670.1 |
| 2 | 93285_at | Cluster Incl AI845584 UI-M-AQ1-adx-c-06-0-UI s1 Mus musculus cDNA, 3 end/clone = UI-M-AQ1-adx-c-06-0-UI/ | 595.4 | P | 493.1 | P | 940.3 | P | 755.7 |
| | | clone_end = 3/gb = AI845584/gi = 5489490/ug = Mm 1791/len = 254/STRA = for | | | | | | | |
| 2 | 93346_at | Cluster Incl M15668.Mus musculus X chromosome-linked phosphoglycerate kinase (pgk-1) mRNA, complete cds/ | 1890.3 | P | 2156.4 | P | 2712.2 | P | 2292.7 |
| | | cds = (22,1275)/gb = M15668/gi = 202422/ug = Mm 188/len = 1653/STRA = for | | | | | | | |
| 2 | 93528_s_at | Cluster Incl AI848050 UI-M-AH1-agp-g-10-0-UI.s1 Mus musculus cDNA, 3 end/clone = UI-M-AH1-agp-g-10-0-UI/ | 327.5 | P | 426.9 | P | 798.5 | P | 626.8 |
| | | clone_end = 3/gb = AI848050/gi = 5491956/ug = Mm 19788/len = 377/STRA = for | | | | | | | |
| 2 | 93728_at | Cluster Incl X62940: Transforming growth factor beta 1 induced transcript 4/cds = (186,617)/gb = X62040/gi = 54928/ | 876.2 | P | 1031.9 | P | 1521.1 | P | 1208.3 |
| | | ug = Mm.20927/len = 1706/STRA = for | | | | | | | |
| 2 | 94048_at | Cluster Incl AW120683: UI-M-BH2.3-anz-c-06-0-UI.s1 Mus musculus cDNA, 3 end/clone = UI-M-BH2.3-anz-c-06-0-UI/ | 399.2 | P | 571.3 | P | 559.8 | P | 516.2 |
| | | clone_end = 3/gb = AW120683/gi = 6095981/ug = Mm 21981/len = 420/STRA = for | | | | | | | |
| 2 | 94052_at | Cluster Incl AB013360 Mus musculus mRNA for DPM2, complete cds/cds = (24,278)/gb = AB013360/gi = 3790360/ | 851.2 | P | 858.3 | P | 964.9 | P | 886 |
| | | ug = Mm 22001/len = 279/STRA = for | | | | | | | |
| 2 | 94236_at | Cluster Incl AI838152 UI-M-AL0-abw-e-06-0-UI a1 Mus musculus cDNA, 3 end/clone = UI-M-AL0-abw-e-06-0-UI/ | 1522.3 | P | 1593.2 | P | 2020.6 | P | 1427.7 |
| | | clone_end = 3/gb = AI838152/gi = 5472365/ug = Mm 22330/len = 451/STRA = for | | | | | | | |
| 2 | 94255_g_at | Cluster Incl AI845237 UI-M-BG0-aht-g-09-0-UI s1 Mus musculus cDNA, 3 end/clone = UI-M-BG0-aht-g-09-0-UI/ | 1055.5 | P | 1080.3 | P | 1497.2 | P | 1433.8 |
| | | clone_end = 3/gb = AI845237/gi = 5489143/ug = Mm 22409/len = 255/STRA = for | | | | | | | |
| 2 | 94261_at | Cluster Incl AI836625. UI-M-AP0-abh-a-12-0-UI.s1 Mus musculus cDNA, 3 end/clone = UI-M-AP0-abh-a-12-0-UI/ | 1531.9 | P | 1390.6 | P | 1914.6 | P | 1524.1 |
| | | clone_end = 3/gb = AI836625/gi = 5470838/ug = Mm 22440/len = 218/STRA = for | | | | | | | |
| 2 | 94300_f_at | Cluster Incl AI839497: UI-M-AN0-acn-b-04-0-UI s1 Mus musculus cDNA, 3 end/clone = UI-M-AN0-acn-b-04-0-UI/ | 788.2 | P | 896.5 | P | 907.5 | P | 875.7 |
| | | clone_end = 3/gb = AI839497/gi = 5473710/ug = Mm 22597/len = 385/STRA = for | | | | | | | |
| 2 | 94309_g_at | Cluster Incl X70853: BM-90/fibulin extracellular matrix glyroprotein/cds = (0,353)/gb = X70853/gi = 396816/ug = Mm.22626/ | 1220.3 | P | 1590.8 | P | 1903 | P | 1144.3 |
| | | len = 448/STRA = for | | | | | | | |
| 2 | 95468_at | Cluster Incl AI850202 UI-M-BG1-aie-b-01-0-UI s1 Mus musculus cDNA, 3 end/clone = UI-M-BG1-aie-b-01-0-UI/ | 271.4 | P | 302.6 | P | 418.1 | P | 214.5 |
| | | clone_end = 3/gb = AI850202/gi = 5494108/ug = Mm 24786/len = 401/STRA = for | | | | | | | |
| 2 | 95471_at | Cluster Incl U22399 Cyclin-dependent kinase Inhibitor 1C (P57)/cds = (137,1144)/gb = U22399/gi = 790249/ug = Mm.2480/ | 3334.3 | P | 4337 | P | 4660.3 | P | 3753.8 |
| | | len = 1462/STRA = for | | | | | | | |
| 2 | 95474_at | Cluster Incl AW123850 UI-M-BH2 1-app-b-03-0-UI s1 Mus musculus cDNA, 3 end/clone = UI-M-BH2 1-app-b-03-0-UI/ | 371.2 | P | 608.7 | P | 639.2 | P | 329.4 |
| | | clone_end = 3/gb = AW123850/gi = 6099380/ug = Mm 24816/len = 347/STRA = for | | | | | | | |
| 2 | 95482_at | Cluster Incl AW124247: UI-M-BH2 1-aph-f-08-0-UI s1 Mus musculus cDNA, 3 end/clone = UI-M-BH2 1-aph-f08-0-UI | 682.2 | P | 746.2 | P | 882.8 | P | 714.8 |
| | | clone_end = 3/gb = AW124247/gi = 6099777/ug = Mm 24870/len = 398/STRA = for | | | | | | | |
| 2 | 96073_at | Cluster Incl U10435 Requiem/cds = (46,1161)/gb = U10435/gi = 606660/ug = Mm.2651/len = 2319/STRA = for | 424.4 | P | 391.2 | P | 429.5 | P | 363.9 |
| 2 | 96076_at | Cluster Incl AW121716: UI-M-BH2 3-aoc-h-05-0-UI s1 Mus musculus cDNA, 3 end/clone = UI-M-BH2.3-aoc-h-05-0-UI/ | 449.9 | P | 549.8 | P | 614.8 | P | 461.7 |
| | | clone_end = 3/gb = AW12176/gi = 6097049/ug = Mm 26558/len = 214/STRA = for | | | | | | | |
| 2 | 96636_at | Cluster Incl AI852649.UI-M-BHO-aiu-h-10-0-UI s1 Mus musculus cDNA, 3 end/clone = UI-M-BH0-aiu-h-10-0-UI/ | 423.1 | P | 369.5 | P | 447.8 | P | 521.8 |
| | | clone_end = 3/gb = AI852649/gi = 5496555/ug = Mm 27270/len = 388/STRA = for | | | | | | | |
| 2 | 97308_at | Cluster Incl AI835409 UI-M-AQ0-aaj-d-08-0-UI s1 Mus musculus cDNA, 3 end/clone = UI-M-AQ0-aaj-d-08-0-UI/ | 236.5 | P | 293.8 | P | 312.9 | P | 246.7 |
| | | clone_end = 3/gb = AI835409/gi = 5469622/ug = Mm 28063/len = 314/STRA = for | | | | | | | |
| 2 | 97813_at | Cluster Incl M61909 Avian reticuloendotheliosis viral (v-rel) oncogene homolog A/cds = (60,1709)/gb = M61909/gi = 200025/ | 1164.9 | P | 1431.3 | P | 1811.5 | P | 1379 |
| | | ug = Mm 28170/len = 2424/STRA = for | | | | | | | |
| 2 | 97859_at | Cluster Incl AA762325 vw59d08 r1 M Mus musculus cDNA, 5 end/clone = IMAGE-1248111/clone_end = 5/gb = AA762325/ | 323 | P | 427 | P | 522.8 | P | 358.3 |
| | | gi = 2812072/ug = Mm 28308/len = 261/STRA = for | | | | | | | |
| 2 | 98926_at | Cluster Incl AI838601 UI-M-AO0-abz-e-11-0-UI s1 Mus musculus cDNA, 3 end/clone = UI-M-AO0-abz-e-11-0-UI/ | 178.4 | P | 228.1 | P | 349.3 | P | 196 |
| | | clone_end = 3/gb = AI838601/gi = 5472814/ug = Mm 28643/len = 322/STRA = for | | | | | | | |
| 2 | 98933_at | Cluster Incl AI849218 UI-M-AJ1-agz-b-02-0-UI s1 Mus musculus cDNA, 3 end/clone = UI-M-AJ1-agz-b-02-0-UI/ | 612.2 | P | 566.1 | P | 574.8 | P | 589.2 |
| | | clone_end = 3/gb = AI849218/gi = 5493124/ug = Mm 28678/len = 450/STRA = for | | | | | | | |
| 2 | 94457_at | Cluster Incl AW049735 UI-M-BH1-ani-g-08-0-UI s1 Mus musculus cDNA, 3 end/clone = UI-M-BH1-ani-g-08-0-UI/ | 603.6 | P | 888.2 | P | 760.7 | P | 667.4 |
| | | clone_end = 3/gb = AW049735/gi = 5910264/ug = Mm 28806/len = 510/STRA = for | | | | | | | |
| 2 | 94482_at | Cluster Incl AF012251.Casein kinase II, alpha 2, polypeptide/cds = (98,1150)/gb = AF012251/gi = 2738574/ug = Mm 28881/ | 1364.9 | P | 1578.2 | P | 1709.6 | P | 1012.2 |
| | | len = 1630/STRA = for | | | | | | | |
| 2 | 94483_at | Cluster Incl AW060442 UI-M-BH1-anj-e-09-0-UI s1 Mus musculus cDNA, 3 end/clone = UI-M-BH1-anj-e-09-0-UI/ | 607.1 | P | 548.8 | P | 538.3 | P | 521.4 |
| | | clone_end = 3/gb = AW060442/gi = 6008193/ug = Mm 28881/len = 471/STRA = for | | | | | | | |
| 2 | 94555_at | Cluster Incl AI845579.UI-M-AQ1-adx-b-12-0-UI s1 Mus musculus cDNA, 3 end/clone = UI-M-AQ1-adx-b-12-0-UI/ | 947.1 | P | 678.9 | P | 1353 | P | 878 |
| | | clone_end = 3/gb = AI845579/gi = 5489485/ug = Mm 29092/len = 254/STRA = for | | | | | | | |
| 2 | 95044_at | Cluster Incl AI844549: UI-M-AJ1-ahf-h-10-0-UI s2 Mus musculus cDNA, 3 end/clone = UI-M-AJ1-ahf-h-10-0-UI/ | 477.6 | P | 497.9 | P | 699.1 | P | 580.3 |
| | | clone_end = 3/gb = AI844549/gi = 5488455/ug = Mm 29120/len = 430/STRA = for | | | | | | | |
| 2 | 95057_at | Cluster Incl AI846938 UI-M-AK1-aey-e-10-0-UI s1 Mus musculus cDNA, 3 end/clone = UI-M-AK1-aey-e-10-0-UI/ | 535 | P | 525.6 | P | 550.5 | P | 454.2 |
| | | clone_end = 3/gb = AI846938/gi = 5490844/ug = Mm 29151/len = 342/STRA = for | | | | | | | |
| 2 | 95081_at | Cluster Incl AI840176 UI-M-AO0-acc-f-04-0-UI s1 Mus musculus cDNA, 3 end/clone = UI-M-AO0-acc-f-04-0-UI/ | 586 | P | 1029.1 | P | 1576.4 | P | 608.9 |
| | | clone_end = 3/gb = AI840176/gi = 5474389/ug = Mm 29253/len = 407/STRA = for | | | | | | | |
| 2 | 95128_at | Cluster Incl AI849611.UI-M-AH1-agr-g-04-0-UI s1 Mus musculus cDNA, 3 end/clone = UI-M-AH1-agr-g-04-0-UI | 470.6 | P | 572.8 | P | 613.1 | P | 485 |
| | | clone_end = 3/gb = AI849611/gi = 5493517/ug = Mm 29412/len = 309/STRA = for | | | | | | | |
| 2 | 95144_at | Cluster Incl AB024984.Mus musculus Sld329 mRNA, complete cds/cds = (134,1246)/gb = AB024984/gi = 5931548/ | 603.5 | P | 718.4 | P | 721 | P | 662.4 |
| | | ug = Mm 29453/len = 1595/STRA = for | | | | | | | |
| 2 | 95161_at | Cluster Incl AW120628 UI-M-BH2 3-anz-a-04-0-UI.s1 Mus musculus cDNA, 3 end/clone = UI-M-BH2 3-anz-a-04-0-UI/ | 728.7 | P | 1011.8 | P | 1241 | P | 738.7 |
| | | clone_end = 3/gb = AW120628/gi = 6095961/ug = Mm.29490/len = 381/STRA = for | | | | | | | |
| 2 | 95653_at | Cluster Incl AI841323 UI-M-AM0-adv-b-03-0-UI s1 Mus musculus cDNA, 3 end/clone = UI-M-AM0-adv-b-03-0-UI/ | 724 | P | 703.6 | P | 682.9 | P | 704.9 |
| | | clone_end = 3/gb = AI841323/gi = 5475538/ug = Mm.29517/len = 449/STRA = for | | | | | | | |
| 2 | 95682_at | Cluster Incl AB026432 Mus musculus mRNA for XPE UV-damaged DNA binding factor, complete cds/cds = (81,3503)/ | 2641.8 | P | 3266.4 | P | 3358 | P | 3036.8 |
| | | gb = AB026432/gi = 5931595/ug = Mm 29623/len = 4171/STRA = for | | | | | | | |
| 2 | 95692_at | Cluster Incl AI852514 UI-M-BH0-aiw-c-06-0-UI s1 Mus musculus cDNA, 3 end/clone = UI-M-BH0-aiw-c-06-0-UI/ | 865.5 | P | 916.3 | P | 966.4 | P | 814.6 |
| | | clone_end = 3/gb = AI852514/gi = 5496420/ug = Mm.29655/len = 298/STRA = for | | | | | | | |
| 2 | 95731_at | Cluster Incl AI843106 UI-M-AK1-aes-e-01-0-UI s1 Mus musculus cDNA, 3 end/clone = .UI-M-AK1-aes-e-01-0-UI/ | 295.2 | P | 341.3 | P | 358.6 | P | 228.4 |
| | | clone_end = 3/gb = AI843106/gi = 5477319/ug = Mm.29742/len = 302/STRA = for | | | | | | | |
| 2 | 96852_at | Cluster Incl AW122197.UI-M-BH2 2-aov-b-080-UI s1 Mus musculus cDNA, 3 end/clone = UI-M-BH2 2-aov-b-08-0-UI/ | 1728.9 | P | 1995.4 | P | 2014.2 | P | 1747.9 |
| | | clone_end = 3/gb = AW122197/gi = 6097654/ug = Mm 30039/len = 436/STRA = for | | | | | | | |
| 2 | 96876_at | Cluster Incl U34259 Mus musculus Golgi 4-transmembrane spanning transporter MTP mRNA, complete cds/ | 3695.5 | P | 3520 | P | 4151.3 | P | 337.4 |
| | | cds = (744,1445)/gb = U34259/gi = 1463119/ug = Mm 30071/len = 1984/STRA = for | | | | | | | |
| 2 | 96926_at | Cluster Incl AA980164: ua31a05.r1 Mus musculus cDNA, 5 end/clone = IMAGE-1348304/clone_end = 5/gb = AA980164/ | 356.1 | P | 449 | P | 542.3 | P | 426.1 |
| | | gi = 3158700/ug = Mm.30162/len = 470/STRA = for | | | | | | | |
| 2 | 97517_at | Cluster Incl AW061222: UI-M-BH1-amj-a-02-0-UI.s2 Mus musculus cDNA, 3 end/clone = UI-M-BH1-amj-a-02-0-UI | 1150.8 | P | 1145.2 | P | 1243.8 | P | 1110.1 |
| | | clone_end = 3/gb = AW061222/gi = 6008973/ug = Mm 3199/len = 143/STRA = for | | | | | | | |
| 2 | 98060_at | Cluster Incl D49733 Lamin A/cds = (0,1997)/gb = D49733/gi = 1838920/ug = Mm.3438 len = 2916/STRA = for | 1433.1 | P | 1728.3 | P | 1639.5 | P | 1526 |
| 2 | 98064_at | Cluster Incl AW125378 UI-M-BH2 3-aqh-b-04-0-UI s1 Mus musculus cDNA, 3 end/clone = UI-M-BH2 3-aqh-b-04-0-UI/ | 1014.4 | P | 799.4 | P | 794 | P | 715.1 |
| | | clone_end = 3/gb = AW125378/gi = 6100908/ug = Mm 34411/len = 447/STRA = for | | | | | | | |
| 2 | 98067_at | Cluster Incl U09507: Cyclin-dependent kinase inhibitor 1A (P21)/cds = (200,679)/gb = U09507/gi = 595302/ug = Mm.34446/ | 1472.4 | P | 1082 | P | 1695.9 | P | 1563 |
| | | len = 855/STRA = for | | | | | | | |
| 2 | 98083_at | Cluster Incl AW049031 UI-M-BH1-amp-g-08-0-UI s1 Mus musculus cDNA, 3 end/clone = UI-M-BH1-amp-g-08-0-UI/ | 488.1 | P | 533.3 | P | 1262.9 | P | 672.2 |
| | | clone_end = 3/gb = AW049031/gi = 5909560/ug = Mm 34576/len = 473/STRA = for | | | | | | | |
| 2 | 98140_at | Cluster Incl X60961 Cadherin 1/cds = (127,2781)/gb = X60961/gi = 55133/ug = Mm 35605/len = 4413/STRA = for | 846 | P | 1042.3 | P | 1110.4 | P | 867.1 |
| 2 | 98579_at | Cluster Incl M28845 Early growth response 1/cds = (489,2090)/gb = M28845/gi = 1325981/ug = Mm 3765/len = 3476/ | 894 | P | 747.4 | P | 2210.3 | P | 1097.5 |
| | | STRA = rev | | | | | | | |
| 2 | 93605_at | Cluster Incl AI851163 UI-M-BH0-akg-f-05-0-UI.s1 Mus musculus cDNA, 3 end/clone = UI-M-BH0-akg-f-05-0-UI/ | 678.6 | P | 665.8 | P | 650.8 | P | 657.9 |
| | | clone_end = 3/gb = AI851163/gi = 5495034/ug = Mm.38433/len = 399/STRA = for | | | | | | | |
| 2 | 99086_g_at | Cluster Incl AI021421 ub 12a08n r1 Mus musculus cDNA, 5 end/clone = IMAGE-1366742/clone_end = 5/gb = AI021421/ | 281.6 | P | 142.4 | P | 208.7 | P | 112.9 |
| | | gi = 3235757/ug = Mm 38976/len = 470/STRA = for | | | | | | | |
| 2 | 99109_at | Cluster Incl M59821 Mouse growth factor-inducible protein (pip92) mRNA, complete cds/cds = (103,768)/gb = M59821/ | 828.1 | P | 918 | P | 2090.3 | P | 1106.5 |
| | | gi = 200354/ug = Mm.399/len = 1515/STRA = for | | | | | | | |
| 2 | 99183_at | Cluster Incl AW123013 UI-M-BH2 1-apb-e-03-0-UI s1 Mus musculus cDNA, 3 end/clone = UI-M-BH2 1-apb-e-03-0-UI/ | 446.9 | P | 609.2 | P | 514.1 | P | 473.8 |
| | | clone_end = 3/gb = AW123013/gi = 6098543/ug = Mm.41840/len = 553/STRA = for | | | | | | | |
| 2 | 99566_at | Cluster Incl L31777 Triosephosphate isomerase/cds = (0,634)/gb = L31777/gi = 953213/ug = Mm 4222/len = 635/STRA = for | 3918 | P | 4928.5 | P | 4853.7 | P | 4822.5 |
| 2 | 99574_at | Cluster Incl AI850887.UI-M-BH0-ajt-g-01-0-UI s1 Mus musculus cDNA, 3 end/clone = UI-M-BH0-ajt-g-01-0-UI/clone_end = 3/ | 463.7 | P | 432.3 | P | 532.6 | P | 421.2 |
| | | gb = AI850887/gi = 5494793/ug = Mm 423/len = 394/STRA = for | | | | | | | |
| 2 | 99624_at | Cluster Incl AW125517.UI-M-BH2 2-aqm-f-07-0-UI s1 Mus musculus cDNA, 3 end/clone = UI-M-BH2 2-aqm-f-07-0-UI | 736.2 | P | 806.3 | P | 890.4 | P | 706.8 |
| | | clone_end = 3/gb = AW125517/gi = 6101047/ug = Mm 43309/len = 464/STRA = for | | | | | | | |
| 2 | 99652_at | Cluster Incl AF109905 Mus musculus major histocompatibility locus class III regions Hsc701 gene, partial cds, smRNP, | 1127.1 | P | 1060.6 | P | 1165.5 | P | 1205.2 |
| | | G7A, NG23, MulS homolog, CLCP, NG24, NG25, and NG26 genes, complete cds, and unknown genes/cds = (0,1676)/ | | | | | | | |
| | | gb = AF109905/gi = 3986751/ug = Mm 4/STRA = for | | | | | | | |
| 2 | 99668_at | Cluster Incl U86405 Mus musculus brain amphiphysin 2 mRNA, complete cds/cds = (249,2015)/gb = U86405/gi = 1914840/ | 1156.1 | P | 1584.4 | P | 2172.9 | P | 1555.2 |
| | | ug = Mm 4383/len = 2506/STRA = for | | | | | | | |
| 2 | 100041_at | Cluster Incl AW124133: UI-M-BH2 1-apr-d-12-0-UI s1 Mus musculus cDNA, 3 end/clone = UI-M-BH2 1-apr-d-12-0-UI/ | 989.9 | P | 1142 | P | 1049.6 | P | 969.2 |
| | | clone_end = 3/gb = AW124133/gi = 6099663/ug = Mm 44226/len = 133/STRA = for | | | | | | | |
| 2 | 100056_at | Cluster Incl X54352: M domesticus MD6 mRNA/cds = (123,1391)/gb = X54352/gi = 49679/ug = Mm 4405/len = 1825/ | 287.3 | P | 325.7 | P | 312.2 | P | 324.7 |
| | | STRA = for | | | | | | | |
| 2 | 100074_at | Cluster Incl AW046723: UI-M-BH1-alt-f-03-0-UI s1 Mus musculus cDNA, 3 end/clone = UI-M-BH1-alf-f-03-0-UI/ | 730.3 | P | 649.5 | P | 823.2 | P | 778.2 |
| | | clone_end = 3/gb = AW046723/gi = 5907292/ug = Mm 45233/len = 220/STRA = for | | | | | | | |
| 2 | 100099_at | Cluster Incl Z14132 Sphingomyelin phosphodiesterase 1, acid lysosomal/cds = (0,1883)/gb = Z14132/gi = 475954/ | 1506.5 | P | 1583.2 | P | 1715.1 | P | 1518.4 |
| | | ug = Mm 4628/len = 1884/STRA = for | | | | | | | |
| 2 | 100133_at | Cluster Incl M27266 Fyn proto-oncogene/cds = (225,1829)/gb = M27266/gi = 193357/ug = Mm.4848/len = 2299/STRA = for | 301.5 | P | 238.2 | P | 415.5 | P | 197.4 |
| 2 | 100155_at | Cluster Incl L57509 Cell adhesion kinase/cds = (106,2841)/gb = L57509/gi = 1161062/ug = Mm.5021/len = 3674/STRA = for | 445.6 | P | 400.1 | P | 652.8 | P | 491.7 |
| 2 | 100561_at | Cluster Incl AW209098: un99g09 x1 Mus musculus cDNA, 3 end/clone = IMAGE-2582080/clone_end = 3/gb = AW209098/ | 611.7 | P | 897.1 | P | 638.9 | P | 838.7 |
| | | gi = 6515038/ug = Mm 56685/len = 486/STRA = for | | | | | | | |
| 2 | 100605_at | Cluster Incl M81086 Mouse skeletal muscle beta tropomyosin mRNA, complete cds/cds = (34,888)/gb = M81086/gi202162/ | 703.9 | P | 854.7 | P | 969.9 | P | 714.4 |
| | | ug = Mm 646/len = 2097/STRA = for | | | | | | | |
| 2 | 100635_at | Cluster Incl L20294 Mus musculus GTP-binding protein (mSara) homologue mRNA, complete cds/cds = (84,680)/ | 1609.8 | P | 1954.3 | P | 2086 | P | 1822.4 |
| | | gb = L20294/gi = 436563/ug = Mm 6698/len = 696/STRA = for | | | | | | | |
| 2 | 101028_l_at | Cluster Incl M15501 Actin, alpha, cardiac/cds = (0,1127)/gb = M15501/gi = 191653/ug = Mm 686/len = 1226/STRA = for | 860.2 | P | 2627 | P | 5006.6 | P | 1566.2 |
| 2 | 101030_at | Cluster Incl X99963: M. musculus rhoB gene/cds = (0,590)/gb = X99963/gi = 1491740/ug = Mm 687/len = 591/STRA = for | 1576.3 | P | 2001.5 | P | 2459.2 | P | 1311.7 |
| 2 | 101069_g_at | Cluster Incl AA656621: vr49a10 s1 Mus musculus cDNA, 5 end/clone = IMAGE-1123962/clone_end = 5/gb = AA656621/ | 339.5 | P | 661.7 | P | 877.7 | P | 372 |
| | | gi = 2592775/ug = Mm 7198/len = 335/STRA = for | | | | | | | |
| 2 | 101070_at | Cluster Incl AW125438 UI-M-BH2 3-aqh-g-08-0-UI s1 Mus musculus cDNA, 3 end/clone = UI-M-BH2 3-aqh-g-08-0-UI/ | 300.5 | P | 296.6 | P | 338.7 | P | 286.3 |
| | | clone_end = 3/gb = AW125438/gi = 6100968/ug = Mm 7198/len = 442/STRA = for | | | | | | | |
| 2 | 101502_at | Cluster Incl X89749.M. musculus mRNA for mTGIF protein/cds = (279,1097)/gb = X89749/gi = 1495713/ug = Mm.8155 | 328.5 | P | 306 | P | 475.3 | P | 399.9 |
| | | len = 1186/STRA = for | | | | | | | |
| 2 | 101554_at | Cluster Incl U57524 Mus musculus I kappa B alpha gene, exons 2-6, partial cds/cds = (0,717)/gb = U57524/gi = 4097334/ | 502.3 | P | 670.6 | P | 3771.8 | P | 1017.9 |
| | | ug = Mm 8884/len = 815/STRA = for | | | | | | | |
| 2 | 101977_at | Cluster Incl AW121873 UI-M-BH2 3-aoi-d-02-0-UI.s1 Mus musculus cDNA, 3 end/clone = UI-M-BH2 3-aoi-d-02-0-UI/ | 990.8 | P | 1067.1 | P | 1100.1 | P | 958.6 |
| | | clone_end = 3/gb = AW121873/gi = 6097247/ug = Mm 95709/len = 491/STRA = for | | | | | | | |
| 2 | 102363_r_at | Cluster Incl U20735 Mus musculus transcription factor junB (junB) gene, 5 region and complete cds/cds = (294,1328)/ | 441 | P | 316.2 | P | 1073.6 | P | 731.6 |
| | | gb = U20735/gi = 965003/ug = Mm.1167/len = 1786/STRA = for | | | | | | | |
| 2 | 102751_at | Cluster Incl M32745. Transforming growth factor, beta 3/cds = (610,1842)/gb = M32745/gi = 201949/ug = Mm 1291/len = 2879/ | 432.2 | P | 449.1 | P | 481.5 | P | 368.4 |
| | | STRA = for | | | | | | | |
| 2 | 102792_at | Cluster Incl U55040 Mus musculus uracil-DNA glycosylase (ung) gene, exon 1, partial cds/cds = (27,311)/gb = U55040/ | 886.7 | P | 851.4 | P | 914.3 | P | 826.2 |
| | | gi-1762315/ug = Mm 1393/len = 312/STRA = for | | | | | | | |
| 2 | 102991_s_at | Cluster Incl AF100956 Mus musculus major histocompatibility locus class II region; Fas-binding protein Daxx (DAXX) gene, | 194.6 | P | 171.4 | P | 209.1 | P | 166.8 |
| | | partial cds; Bing1 (BING1), tapasin (tapasin), RalGDS-like factor (RLF), KE2 (KE2), BING4 (BING4), beta1,3-galactosyl | | | | | | | |
| | | transferase (be/STRA = for | | | | | | | |
| 2 | 102996_at | Cluster Incl U80227: Mus musculus ELL homolog mRNA, complete cds/cds = (5,1813)/gb = U80227/gi = 1947072/ | 308 | P | 322.1 | P | 548.6 | P | 361 |
| | | ug = Mm. 1552/len = 3023/STRA = for | | | | | | | |
| 2 | 103031_g_at | Cluster Incl L31397: Dynamin/cds = (73,2658)/gb = L31397/gi = 487858/ug = Mm.16080/len = 3924/STRA = rev | 562.8 | P | 856 | P | 734.7 | P | 355 |
| 2 | 103069_at | Cluster Incl AI853682 UI-M-BH0-ajq-h-03-0-UI s1 Mus musculus cDNA, 3 end/clone = UI-M-BH0-ajq-h-03-0-UI/ | 215.4 | P | 354.2 | P | 367.9 | P | 205.6 |
| | | clone_end = 3/gb = AI853682/gi = 5497628/ug = Mm 16813/len = 425/STRA = for | | | | | | | |
| 2 | 103098_at | Cluster Incl AW045765 UI-M-BH1-akt-c-06-0-UI s1 Mus musculus cDNA, 3 end/clone = UI-M-BH1-akt-c-06-0-UI/ | 590.8 | P | 874.3 | P | 975.5 | P | 808.9 |
| | | clone_end = 3/gb = AW045765/gi = 5906294/ug = Mm. 17533/len = 338/STRA = for | | | | | | | |
| 2 | 103343_at | Cluster Incl AI845815 UI-M-AO1-aeg-b-02-0-UI S1 Mus musculus cDNA, 3 end/clone = UI-M-AO1-aeg-b-02-0-UI/ | 232.1 | P | 290.7 | P | 437.7 | P | 255.5 |
| | | clone_end = 3/gb = AI845815/gi = 5489721/ug = Mm 18228/len = 367/STRA = for | | | | | | | |
| 2 | 103636_at | Cluster Incl AI840971 UI-M-AK0-adk-e-02-0-UI s1 Mus musculus cDNA, 3 end/clone = UI-M-AK0-adk-e-02-o-UI/ | 516.6 | P | 580.8 | P | 443.5 | P | 374.5 |
| | | clone_end = 3/gb = AI840971/gi = 5475184/ug = Mm 20324/len = 389/STRA = for | | | | | | | |
| 2 | 104094_at | Cluster Incl Y08361 M musculus mRNA for RIL protein/cds = (36,1028)/gb = Y08361/gi = 1585268/ug = Mm.21830/len = 1135/ | 89.4 | P | 175.8 | P | 233 | P | 151.5 |
| | | STRA = for | | | | | | | |
| 2 | 104116_at | Cluster Incl AW124049 UI-M-BH2 1-apn-e-03-0-UI s1 Mus musculus cDNA, 3 end/clone = UI-M-BH2 1-apn-e-03-0-UI/ | 260.5 | P | 352.6 | P | 512.3 | P | 200.1 |
| | | clone_end = 3/gb = AW124049/gi = 6099579/ug = Mm 21965/len = 382/STRA = for | | | | | | | |
| 2 | 104149_at | Cluster Incl AI642048 ub75b05.x1 Mus musculus cDNA, 3 end/clone = IMAGE-1383537/clone_end = 3/gb = AI642048/ | 384.2 | P | 413.6 | P | 2097.2 | P | 661.7 |
| | | gi = 4720523/ug = Mm 22188/len = 434/STRA = for | | | | | | | |
| 2 | 104286_at | Cluster Incl AA795541.vq96e09 r1 Mus musculus cDNA, 5 end/clone = IMAGE-1110184/clone_end = 5/gb = AA795541/ | 631 | P | 478.2 | P | 861. 1 | P | 428.6 |
| | | gi = 2858496/ug = Mm 22260/len = 393/STRA = for | | | | | | | |
| 2 | 104302_f_at | Cluster Incl AI851085 UI-M-BH0-akg-b-04-0-UI.s1 Mus musculus cDNA,3 end/clone = UI-M-BH0-akg-b-04-0-UI/ | 463.9 | P | 489.5 | P | 615.6 | P | 600.3 |
| | | clone_end = 3/gb = AI851085/gi = 5494991/ug = Mm 22353/len = 486/STRA = for | | | | | | | |
| 2 | 104352_at | Cluster Incl AI838366 UI-M-AI0-aak-f-10-0-UI.s2 Mus musculus cDNA, 3 end/clone = UI-M-AI0-aak-f-10-0-UI/clone_end = 3/ | 956.8 | P | 1048.6 | P | 1094.4 | P | 975.5 |
| | | gb = AI838366/gi = 5472579/ug = Mm 22572/len = 319/STRA = for | | | | | | | |
| 2 | 104533_at | Cluster Incl AA764261 W49f08.r1 Mus musculus cDNA, 5 end/clone = IMAGE-1225767/clone_end = 5/gb = AA764261/ | 1110.5 | P | 1029.1 | P | 1437.2 | P | 1254 |
| | | gi = 2811783/ug = Mm 2322/len = 320/STRA = for | | | | | | | |
| 2 | 104545_at | Cluster Incl U51866 Mus musculus casein kinase II alpha subunit mRNA, complete cds/cds = (203,1378)/gb = U51866/ | 190.5 | P | 431 | P | 531.1 | P | 245.3 |
| | | gi = 1262299/ug = Mm 23692/len = 1552/STRA = for | | | | | | | |
| 2 | 104598_at | Cluster Incl X61940: Mouse mRNA for a growth factor-inducible immediate early gene (3CH134)/cds = (139,1242)/ | 727.8 | P | 895.1 | P | 255.7 | P | 1194.5 |
| | | gb = X61940/gi = 49735/ug = Mm 2404/len = 1933/STRA = for | | | | | | | |
| 2 | 104711_at | Cluster Incl AW122109 UI-M-BH2 2-aor-a-06-0-UI s1 Mus musculus cDNA, 3 end/clone = UI-M-BH2 2-aor-a-06-0-UI/ | 536.7 | P | 568.5 | P | 544.9 | P | 525 |
| | | clone_end = 3/gb = AW122109/gi = 6097524/ug = Mm 24437/len = 352/STRA = for | | | | | | | |
| 2 | 94378_at | Cluster Incl U94828 Mus musculus retinally abundant regulator of G-protein signaling mRGS-r (RGS-r) mRNA, complete | 423.1 | P | 434.5 | P | 563.2 | P | 358.7 |
| | | cds/cds = (109,714)/gb = U94828/gi = 2605641/ug = Mm 2555/len = 2250/STRA = for | | | | | | | |
| 2 | 94957_at | Cluster Incl AW121154: UI-M-BH2 3-aoh-f-12-0-UI s1 Mus musculus cDNA, 3 end/clone = UI-M-BH2 3-aoh-f-12-0-UI/ | 121.4 | P | 190.2 | P | 198.1 | P | 97.4 |
| | | clone_end = 3/gb = AW121154/gi = 6096487/ug = Mm 27167/len = 421/STRA = for | | | | | | | |
| 2 | 96128_at | Cluster Incl X69026: PCTAIRE-motif protein kinase 3/cds = (318,1673)/gb = X69026/gi = 53612/ug = Mm 28130/len = 2997/ | 357.6 | P | 394.1 | P | 400.6 | P | 216.9 |
| | | STRA = for | | | | | | | |
| 2 | 96132_at | Cluster Incl AB023957 Mus musculus EIG 180 mRNA for ethanol induced gene product, complete cds/cds = (140,481)/ | 1227.6 | P | 1338.7 | P | 1374.8 | P | 845.9 |
| | | gb = AB023957/gi = 6468203/ug = Mm.28143/len = 728/STRA = for | | | | | | | |
| 2 | 96147_at | Cluster Incl AW125070 UI-M-BH2 1-apx-d-08-0-UI s1 Mus musculus cDNA, 3 end/clone = UI-M-BH2 1-apx-d-08-0-UI/ | 203.2 | P | 191.1 | P | 305 | P | 315.2 |
| | | clone_end = 3/gb = AW125070/gi = 6100600/ug = Mm 28230/len = 438/STRA = for | | | | | | | |
| 2 | 96723_f _at | Cluster Incl AW122911 UI-M-BH2-1-apa-f-09-0-UI s1 Mus musculus cDNA, 3 end/clone = UI-M-BH2 1-apa-f-09-0-UI/ | 617 | P | 550.9 | P | 635.6 | P | 469.4 |
| | | clone_end = 3/gb = AW122911/gi = 6098441/ug = Mm 28829/len = 366/STRA = for | | | | | | | |
| 2 | 96742_at | Cluster Incl AA717826 vt98g09 r1 Mus musculus cDNA, 5 end/clone = IMAGE-1179232/clone_end = 5/gb = AA717826/ | 492.3 | P | 855.6 | P | 991.2 | P | 652.8 |
| | | gi = 2730100/ug = Mm 28935/len = 255/STRA = for | | | | | | | |
| 2 | 96797_s_at | Cluster Incl AW122681 UI-M-BH2 2-aot-a-02-0-UI s1 Mus musculus cDNA, 3 end/clone = UI-M-BH2 2-aot-a-02-0-UI/ | 797.6 | P | 870.6 | P | 951 | P | 778.9 |
| | | clone_end = 3/gb = AW122681/gi = 6098211/ug = Mm 29170/len = 349/STRA = for | | | | | | | |
| 2 | 96813_f_at | Cluster Incl AI852973 UI-M-BH0-aiz-g-09-0-UI s1 Mus musculus cDNA, 3 end/clone = UI-M-BH0-aiz-g-09-0-UI/ | 778.8 | P | 713 | P | 827.2 | P | 764.7 |
| | | clone_end = 3/gb = AI852973/gi = 5496879/ug = Mm 29297/len = 375/STRA = for | | | | | | | |
| 2 | 97347_at | Cluster Incl AA838868 vw78c04.r1 Mus musculus cDNA, 5 end/clone = IMAGE-1261062/clone_end = 5/gb = AA838868/ | 514 | P | 509.6 | P | 676.1 | P | 368.6 |
| | | gi = 2914963/ug = Mm 29618/len = 493/STRA = rev | | | | | | | |
| 2 | 97349_at | Cluster Incl AA727410 vu99g08 r1 Mus museulus cDNA, 5 end/clone = IMAGE-1210334/clone_end = 5/gb = AA727410/ | 605.6 | P | 771.5 | P | 941.4 | P | 768.7 |
| | | gi = 2745117/ug = Mm.2962/len = 506/STRA = for | | | | | | | |
| 2 | 97366_at | Cluster Incl AI851024 UI-M-BH0-ajv-d-07-o-UI s1 Mus musculus cDNA, 3 end/clone = UI-M-BH0-ajv-d-07-0-UI/ | 359.9 | P | 319.1 | P | 654.5 | P | 393.7 |
| | | clone_end = 3/gb = AI851024/gi = 5494930/ug = Mm.29689/len = 132/STRA = for | | | | | | | |
| 2 | 97370_at | Cluster Incl AI552922 vg73I08 y1 Mus musculus cDNA, 5 end/clone = IMAGE-871623/clone_end = 5/gb = AI552922/ | 327.2 | P | 332 | P | 313.4 | P | 351.1 |
| | | gi = 4485285/ug = Mm 29694/len = 222/STRA = fof | | | | | | | |
| 2 | 97405_at | Cluster Incl M28489: Mus musculus (dona Mu6a) ribosomal pratotein S6 kinase (rsk) mRNA, complete cds/cds = (151,2325)/ | 644.6 | P | 597.8 | P | 660.4 | P | 509.9 |
| | | qb = M28489/gi = 556321/ug = Mm 29910/len = 3081/STRA = for | | | | | | | |
| 2 | 97423_at | Cluster Incl AI839819 UI-M-AN0-acq-c-03-0-UI s1 Mus musculus cDNA, 3 end/clone = UI-M-AN0-acq-c-03-0-UI/ | 342 | P | 490.4 | P | 607.8 | P | 328.7 |
| | | clone_end = 3/gb = AI839819/gi = 5473997/ug = Mm.30006/len = 305/STRA = rev | | | | | | | |
| 2 | 97429_at | Cluster Incl AW048113 UI-M-BH1-alq-d-12-0-UI s1 Mus musculus cDNA, 3 end/clone = UI-M-BH1-alq-d-12-0-UI/ | 271.3 | P | 369.9 | P | 559.7 | P | 325.5 |
| | | clone_end = 3/gb = W048113/gi = 5908642/ug = Mm.30079/len = 395/STRA = for | | | | | | | |
| 2 | 97921_at | Cluster Incl AI846279 UI-M-AK1-aez-e-04-0-UI s1 Mus musculus cDNA, 3 end/clone = UI-M-AK1-aez-e-04-0-UI/ | 761.8 | P | 698.4 | P | 978.2 | P | 656 |
| | | clone_end = 3/gb = AI846279/gi = 5490185/ug = Mm 30145/len = 409/STRA = for | | | | | | | |
| 2 | 97943_at | Cluster Incl AI747133 ui12a02.x1 Mus musculus cDNA, 3 end/clone = IMAGE-2076554/clone_end = 3/gb = AI747133/ | 612.9 | P | 830 | P | 1090.7 | P | 805.7 |
| | | gi = 5125397/ug = Mm 30290/len = 560/STRA = for | | | | | | | |
| 2 | 97982_at | Cluster Incl AA260139 va37a08 r1 Mus musculus cDNA, 5 end/clone = IMAGE-733526/clone_end = 5/gb = AA260139/ | 267.1 | P | 471.6 | P | 421.8 | P | 251.9 |
| | | gi = 1896624/ug = Mm 3127/len = 303/STRA = for | | | | | | | |
| 2 | 98999_at | Cluster Incl AA606587.vm86b01 r1 Mus musculus cDNA, 5 end/clone = IMAGE-1005097/clone_end = 5/gb = AA606587/ | 239.1 | P | 317.7 | P | 265.6 | P | 259.6 |
| | | gi = 245521/ug = Mm 38151/len = 559/STRA = for | | | | | | | |
| 2 | 99042_s_at | Cluster Incl U66918: Short stature homeobox 2/cds = (215,1210)/gb = U66918/gi = 1616757/ug = Mm.39093/len = 1603/ | 324.9 | P | 360.6 | P | 318.3 | P | 249.7 |
| | | STRA = for | | | | | | | |
| 2 | 99052_at | Cluster Incl D76432: Transcriptrion factor 8/cds = (19,3372)/gb = D76432/gi = 1027499/ug = Mm 3929/len = 5214/STRA = rev | 238.1 | P | 351.9 | P | 353.1 | P | 283.4 |
| 2 | 99475_at | Cluster Incl UB8327: Mus musculus suppressor of cytokine signalling-2 (SOCS-2) mRNA, complete cds/cds = (222,818)/ | 287.1 | P | 369.9 | P | 500.8 | P | 308.5 |
| | | gb = U88327/gi = 2245385/ug = Mm.4132/len = 1121/STRA = for | | | | | | | |
| 2 | 99978_s_at | Cluster Incl D83073 Mouse mRNA for p38b, complete cds/cds = (229,1311)/gb = D83073/gi = 1181606/ug = Mm 4437/ | 865.8 | P | 1200.6 | P | 1215.9 | P | 968.6 |
| | | len = 3352/STRA = for | | | | | | | |
| 2 | 100005_at | Cluster Incl X92346 Tnf receptor associated factor 4/cds = (130,1542)/gb = X92346/gi = 1041445/ug = Mm 4521/len:2043/ | 614.6 | P | 639 | P | 708.9 | P | 669.4 |
| | | STRA = for | | | | | | | |
| 2 | 100017_at | Cluster Incl U68267: Mus musculus myosin binding protein H (MyBP-H) mRNA, complete cds/cds = (50,1501)/gb = U68267/ | 333.8 | P | 439.2 | P | 447.1 | P | 382.1 |
| | | gi = 1546812/ug = Mm 4562/len = 1850/STRA = for | | | | | | | |
| 2 | 100022_at | Cluster Incl D89613: Cytokine inducible SH2-containing protein/cds = UNKNOWN/gb = D89613/gi = 2554633/ug = Mm 4592/ | 982 | P | 960.5 | P | 941.9 | P | 596.3 |
| | | len = 1010/STRA = for | | | | | | | |
| 2 | 100024_at | Cluster Incl AI641895vq44e08 y1 Mus musculus cDNA, 5 end/clone = IMAGE-1097126/clone_end = 5/gb = AI641895/ | 504.2 | P | 435.6 | P | 535.1 | P | 424.4 |
| | | gi = 4720370/ug = Mm 46014/len = 413/STRA = for | | | | | | | |
| 2 | 100407_at | Cluster Incl L38580 Galanin/cds = (0,374)/gb = L38580/gi = 927115/ug = Mm.4655/len = 553/STRA = for | 468.9 | P | 454.4 | P | 497.6 | P | 430.8 |
| 2 | 100441_s_at | Cluster Incl X69064: Ankyrin 1, erythroid/cds = (0,731)/gb = X69064/gi = 311818/ug = Mm 4789/len = 1704/STRA = for | 53.1 | P | 97.2 | P | 159.6 | P | 65.4 |
| 2 | 100488_at | Cluster Incl M24119: Acid beta glucosidase/cds = (86,1633)/gb = M24119/gi = 193449/ug = Mm.5031/len = 1684/STRA = for | 688.4 | P | 670.5 | P | 855.9 | P | 606.4 |
| 2 | 100877_at | Cluster Incl AI194274 ul14c07 r1 Mus musculus cDNA, 5 end/clone = IMAGE-1851276/clone_end = 5/gb = AI194274/ | 585.8 | P | 939.9 | P | 973.5 | P | 504.9 |
| | | gi = 3745481/ug = Mm 53/len = 480/STRA = for | | | | | | | |
| 2 | 100899_s_at | Cluster Incl AI265210 ul97a10.x1 Mus musculus cDNA, 3 end/clone = IMAGE-1890330/clone_end = 3/gb = AI265210/ | 640.8 | P | 603.9 | P | 747.1 | P | 495.6 |
| | | gi = 3883368/ug = Mm 57157/len = 485/STRA = for | | | | | | | |
| 2 | 100928_at | Cluster Incl X75285 Fibulin 2/cds = (50,3715)/gb = X75285/gi = 437046/ug = Mm 6120/len = 4377/STRA = for | 818 | P | 856.7 | P | 985.1 | P | 609.1 |
| 2 | 100960_g_at | Cluster Incl X99921 M musculus mRNA for S100 calcium-binding protein A13/cds = (60,356)/gb = X99921/gi = 1694840/ | 724.4 | P | 763.6 | P | 792.8 | P | 821.5 |
| | | ug = Mm 6523/len = 440/STRA = for | | | | | | | |
| 2 | 101357_at | Cluster Incl X14971 Mouse mRNA for alpha-adaptin (A)/cds = (205,3138)/gb = X14971/gi = 49877/ug = Mm.6877/len = 3496/ | 542.7 | P | 227.4 | P | 686.5 | P | 444 |
| | | STRA = for | | | | | | | |
| 2 | 102209_at | Cluster Incl AF087434.Mus musculus transcription factor NF-ATc isoform a (NF-ATca) mRNA, complete cds/cds = (0,2153/) | 123.4 | P | 87.5 | P | 207 | P | 56.1 |
| | | gb = AF087434/gi = 3643194/ug = Mm 10085/len = 3469/STRA = for | | | | | | | |
| 2 | 102213_at | Cluster Incl AF051945 Mus musculus Xin mRNA, complete cds/cds = (140,5173)/gb = AF051945/gi = 2970645/ | 206.7 | P | 211.8 | P | 514.8 | P | 302.3 |
| | | ug = Mm 10117/len = 5800/STRA = for | | | | | | | |
| 2 | 102216_at | Cluster Incl Y14334 Arachidonale 12-lipoxygenase, pseudogene 2/cds = (175,2280)/gb = Y14334/gi = 3043452/ | 278.5 | P | 339 | P | 689.1 | P | 367.2 |
| | | ug = Mm 10143/len = 2347/STRA = for | | | | | | | |
| 2 | 102283_at | Clustar Incl U05245 T-cell lymphoma invasion and metastasis 1/cds = (506,5281)/gb = U05245/gi = 497638/ug = Mm.1211/ | 558.8 | P | 508.4 | P | 723.8 | P | 527.5 |
| | | 1en = 7292/STRA = for | | | | | | | |
| 2 | 102683_at | Cluster Incl U76009 Zinc transporter 3/cds = (0,1025)/gb = U76009/gi = 1763379/ug = Mm.1396/len = 1026/STRA = for | 1508.4 | P | 1481.1 | P | 1538 | P | 1484 |
| 2 | 102737_at | Cluster Incl U35233 Endothelin 1/cds = (30,638)/gb = U35233/gi = 1244501/ug = Mm 14543/len = 1770/STRA = for | 238.3 | P | 347.7 | P | 284.6 | P | 174.3 |
| 2 | 102955_at | Cluster Incl U83148 Mus musculus NFIL3/E4BP4 transaction factor mRNA, complete cds/cds = (0,1388)/gb = U83148/ | 171.5 | P | 147.5 | P | 253.5 | P | 222.7 |
| | | gi = 2076877/ug = Mm 1755/len = 1389/STRA = for | | | | | | | |
| 2 | 103215_g_at | Cluster Incl AW048883 UI-M-BH1-amf-f-01-0-UI.s1 Mus musculus cDNA, 3 end/clone = UI-M-BH1-amf-l-01-0-UI/ | 628.6 | P | 731.9 | P | 876.4 | P | 657.3 |
| | | clone_end = 3/gb = AW048883/gi = 5909412/ug = Mm 1980/len = 489/STRA = for | | | | | | | |
| 2 | 103504_at | Cluster Incl AI837107.UI-M-AK0-adc-d-11-0-UI s1 Mus muaculus cDNA, 3 end/clone = UI-M-AK0-adc-d-11-0-UI/ | 307.9 | P | 299.5 | P | 410.5 | P | 315.3 |
| | | clone_end = 3/gb = AI637107/gi = 5471320/ug = Mm 22438/len = 460/STRA = for | | | | | | | |
| 2 | 103736_at | Cluster Incl AI837786: UI-M-AK0-adl-e-02-0-UI s1 Mus musculus cDNA, 3 end/clone = UI-M-AK0-adl-e-02-0-UI/ | 284.1 | P | 369.4 | P | 366.1 | P | 262.9 |
| | | clone_end = 3/gb = AI837786/gi = 5471999/ug = Mm24385/len = 412/STRA = for | | | | | | | |
| 2 | 104000_at | Cluster lncl AI181346.ub83d07.r1 Mus musculus cDNA, 5 end/clone = IMAGE-1395085/clone_end-5/gb = AM81346/ | 984.7 | P | 1220.4 | P | 1149.2 | P | 1013.7 |
| | | gi = 3731984/ug = Mm 26580/len = 474/STRA = for | | | | | | | |
| 2 | 104155_f_at | Cluster Incl U19118 Mus musculus transcription factor LRG-21 mRNA, complete cds/cds = (167,712)/gb = U19118/ | 274.9 | P | 248.3 | P | 1541.4 | P | 258.6 |
| | | gi = 633751/ug = .Mm2706/len = 1897/STRA = for | | | | | | | |
| 2 | 104435_at | Cluster Incl AI844072: vu70c06 x1 Mus musculua cDNA, 3 end/clone = IMAGE-1196746/clone_end = 3/gb = AI644072/ | 838 | P | 1006.8 | P | 1212.8 | P | 961.3 |
| | | gi = 4722547/ug = Mm 29257/len = 460/STRA = for | | | | | | | |
| 2 | 104477_at | Cluster Incl AW047643: UI-M-BH1-alo-a-02-0-Ul.s1 Mus musculus cDNA. 3 end/clone = UI-M-BH1-alo-a-02-0-UI/ | 133.9 | P | 475.2 | P | 474 | P | 126.4 |
| | | clone_end = 3/gb = AW047643/gl = 5908172/ug = Mm 29940/len = 375/STRA = for | | | | | | | |
| 2 | 92185_at | Cluster Incl AI846023.UI-M-AP1-agl-g-02-0-Ul s1 Mus musculus cDNA, 3 end/clone = UI-M-AP1-agl-g-02-0-UI/ | 483.3 | P | 403.4 | P | 486.8 | P | 470.7 |
| | | clone_end = 3/gb = AI846023/gi = 5489929/ug = Mm 3388/len = 482/STRA = for | | | | | | | |
| 2 | 92216_at | Cluster Incl AF015260 Mus musculus Smad7 mRNA, complele cds/cds = (0,1280)/gb = AF015260/gi = 2460039/ | 199.4 | P | 98.5 | P | 213.4 | P | 155 |
| | | ug = Mm 34407/len = 1281/STRA = for | | | | | | | |
| 2 | 92220_s_at | Cluster Incl U60884 Mus musculus SH3P9 mRNA, complele cds/cds = (178,1482)/gb = U60884/gi = 1438562/ug = Mm.34445/ | 337.6 | P | 458.3 | P | 760.7 | P | 619.5 |
| | | len = 1531/STRA = for | | | | | | | |
| 2 | 92325_at | Cluster Incl AI326889 mj61doe.x1 Mus musculus cDNA, 3 end/clone = IMAGE-480587/clone_end = 3/gb = AI326889/ | 294.7 | P | 241.4 | P | 356.9 | P | 199.8 |
| | | gi = 4061318/ug = Mm 38494/len = 494/STRA = for | | | | | | | |
| 2 | 92352_at | Cluster Incl AF108021.Mus musculus lysophospholipid receptor B3 (Ipb3) gene, complete cds/cds = (0,1136)/gb = AF108021/ | 540.5 | P | 453.9 | P | 535.9 | P | 473.9 |
| | | gi = 4324652/ug = Mm 39366/len.1137/STRA = for | | | | | | | |
| 2 | 92376_at | Cluster Incl U71205 Mus musculus ril mRNA, complete cds/cds = (80,739)/gb = U71205/gi = 1656004/ug = Mm.4009/ | 797.2 | P | 875.2 | P | 822.5 | P | 636 |
| | | len = 1079/STRA = for | | | | | | | |
| 2 | 92484_at | Cluster Incl Y15907 Mus musculus mRNA for myc-intron-binding proteln-1/cds = (84,7376)/gb = Y15907/gi = 2706548/ | 151.9 | P | 137.3 | P | 146.6 | P | 103.5 |
| | | ug = Mm 42157/len = 8095/STRA = rev | | | | | | | |
| 2 | 92730_at | Cluster Incl L07284 Heparin binding epidermal growth factor-like growth factor/cds = (225,851)/gb = L07264/gl = 192999/ | 166.8 | P | 150.3 | P | 475.8 | P | 526.2 |
| | | ug = Mm 4661/len1000/STR = for | | | | | | | |
| 2 | 92968_at | Cluster Incl D87902: Mouse mRNA for ARF5, complete cds/cds = (49,591)/gb = D87902/gi = 1565214/ug = Mm.4996/len = 933/ | 986.8 | P | 1194.6 | P | 1397.1 | P | 1153.9 |
| | | STRA = for | | | | | | | |
| 2 | 93187_at | Cluster Incl AW048347: UI-M-BH1-akk-e-03-0-Ul.s1 Mus musculus cDNA, 3 end/clone = Ul-M-BH1-akk-e-03-0-Ul/ | 510.9 | P | 618.4 | P | 818.7 | P | 509.7 |
| | | clone end = 3/gb = AW048347/gl = 5908876/ug = Mm 5510/len = 458/STRA = for | | | | | | | |
| 2 | 93197_at | Cluster Incl AF109905 Mus musculus major histocompatibility locus class III regions Hsc70t gene, partial cds; smRNP, | 1564.2 | P | 1311.9 | P | 2229.9 | P | 1088.5 |
| | | G7A, NG23, MutS homolog, CLCP, NG24, NG25, and NG26 genes, complete cds, and unknown genes/cds = (0,380)/ | | | | | | | |
| | | gb = AF 109905/gi = 3986751/ug = Mm 56/STRA = for | | | | | | | |
| 2 | 93434_at | Cluster Incl AF026074 Mus musculus sulfotransferase-related protein (SULT-X1) mRNA, complete cds/cds = (88,963)/ | 160.1 | P | 159.2 | P | 163.3 | P | 121.1 |
| | | gb = AF026074/gi = 2570901/ug = Mm 6562/len = 1173/STRA = for | | | | | | | |
| 2 | 93852_at | Cluster Incl AW045443: UI-M-BH1-akr-f-12-0-UI s1 Mus musculus cDNA, 3 end/clone = UI-M-BM1-akr-f-12-0-UI/ | 429.9 | P | 400.3 | P | 471.3 | P | 441.8 |
| | | clone_end = 3/gb = AW045443/gi = 5906012/ug = Mm 87279/len = 292/STRA = for | | | | | | | |
| 2 | 93922_g_at | Cluster Incl AI573601-ud46a12.y1 Mus musculus cDNA, 5 end/clone = IMAGE-1448926/clone_end = 5/gb = AI573601/ | 570.2 | P | 587 | P | 715.3 | P | 595.7 |
| | | gi = 4536975/ug = Mm 89855/len = 535/STRA = for | | | | | | | |
| 2 | 93965_r_at | Cluster Incl AF038995 Mus musculus putative RNA helicase RCK mRNA, partial cds/cds = (0,1359)/gb = AF038995/ | 371.4 | P | 178.6 | P | 394.1 | P | 587 |
| | | gi = 2745893/ug = Mm 97095/len = 1360/STRA = for | | | | | | | |
| 2 | 95286_at | Cluster Incl D14077 Clusterin/cds = (91,1437)/gb = D14077/gi = 220601/ug = Mm 18700/len = 1663/STRA = for | 645.7 | P | 687.5 | P | 982.3 | P | 743.3 |
| 2 | 95345_at | Cluster Incl AJ012160 Mus musculus 5T4 oncofetal trophoblast glycoprotein gene/cds = (328,1608)/gb = AJ012160/ | 197.2 | P | 219 | P | 304.8 | P | 289.6 |
| | | gi = 3805948/ug = Mm.20864/len = 2329/STRA = for | | | | | | | |
| 2 | 95383_at | Cluster Incl AI415418 mc4Be08 x1 Mus musculus cDNA, 3 end/clone = IMAGE-351782/clone_end = 3/gb = AI415418/ | 830.3 | P | 884.4 | P | 883.8 | P | 804.4 |
| | | gi = 4258922/ug = Mm 23082/len = 455/STRA = rev | | | | | | | |
| 2 | 95989_at | Cluster Incl AI448172 mo64107 x1 Mus musculus cDNA, 3 end/clone = IMAGE-558373/clone_end = 3/gb = AI448172/ | 896.1 | P | 541.9 | P | 804.9 | P | 635.5 |
| | | gi = 4309126/ug = Mm 25049/len = 373/STRA = for | | | | | | | |
| 2 | 97114_at | Cluster Incl U57999 Prosaposin/cds = (25,1698)/gb = U57999/gi = 1381581/ug = Mm 3363/len = 1961/STRA = for | 1742.1 | P | 1790.2 | P | 2212.6 | P | 1655.1 |
| 2 | 97132_at | Cluster Incl AA189890 mu55b11 r1 Mus musculus cDNA,5 end/clone = IMAGE-643293/clone_end = 5/gb = AA189890/ | 662 | P | 868.8 | P | 923.1 | P | 894.8 |
| | | gi = 1776532/ug = Mm 34876/len = 344/STRA = rev | | | | | | | |
| 2 | 97165_r_at | Cluster Incl C77278 C77278 Mus musculus cDNA, 3 end/clone = J0028D08/clone_end = 3/gb = C77278/gi = 2517608/ | 158 | P | 197.7 | P | 250.1 | P | 202.6 |
| | | ug = Mm 35523/len = 591/STRA = rev | | | | | | | |
| 2 | 97193_at | Cluster Incl AA575098 vh82h08 r1 Mus musculus cDNA, 3 end/clone = IMAGE-893535/clone_end = 3/gb = AA575098/ | 131.5 | P | 81.8 | P | 236.8 | P | 122.1 |
| | | gi = 2349724/ug = Mm 36566/len = 336/STRA = rev | | | | | | | |
| 2 | 99367_at | Cluster Incl AA434661 ve16g03 r1 Mus musculus cDNA, 5 end/clone = IMAGE-818356/clone_end = 5/gb = AA434661/ | 64.9 | P | 26.5 | P | 106.3 | P | 49.9 |
| | | gi = 2139575/ug = Mm 56829/len = 320/STRA = for | | | | | | | |
| 2 | 99399_at | Cluster Incl U89486 Mus musculus agouti-related protein (Agrp) gene, complete cds/cds = (0,395)/gb = U89486/gi = 2351716/ | 316.4 | P | 423 | P | 525.8 | P | 384.6 |
| | | ug = Mm 56995/len = 396/STRA = for | | | | | | | |
| 2 | 99851_at | Cluster Incl AA172988 ms58e02 r1 Mus musculus cDNA, 5 end/clone = IMAGE-615770/clone_end = 5/gb = AA172988/ | 735.2 | P | 839.4 | P | 821.6 | P | 669.6 |
| | | gi = 1752041/ug = Mm 6436/len = 344/STRA = for | | | | | | | |
| 2 | 95215_f_at | Cluster Incl D50527.Mouse mRNA for TI-225, complete cds/cds = (37,417)/gb = D50527/gi = 1167509/ug = Mm 331/len = 498/ | 3154.8 | P | 3117.6 | P | 3379.2 | P | 3192.2 |
| | | STRA = for | | | | | | | |
| 2 | 92830_s_at | Cluster Incl X14678 Zinc finger protein 36/cds = (756,1307)/gb = X14678/gi = 54803/ug = Mm.1298/len = 2282/STRA = for | 86.3 | P | 478.6 | P | 1250.3 | P | 227.8 |
| 2 | 92838_at | Cluster Incl L33726 Fascin 1/cds = (78,1559)/gb = L33726/gi = 497774/ug = Mm.13194/len = 2634/STRA = for | 1279.8 | P | 1289.4 | P | 1506.1 | P | 1318.6 |
| 2 | 92874_f_at | Cluster Incl AF071316 Mus musculus COP9 complex subunit 7a (COPS7a) mRNA, complete cds/cds = (158,985)/ | 339.5 | P | 332.3 | P | 348.2 | P | 384.4 |
| | | gb = AF071316/gi = 3309173/ug = Mm.1444/len = 1779/STRA = for | | | | | | | |
| 2 | 94063_at | Cluster Incl X85991 M musculus mRNA for semaphorin B/cds = (65,2347)/gb = X85991/gi = 854325/ug = Mm.22061/ | 310.3 | P | 360.5 | P | 439.9 | P | 300.9 |
| | | len = 3046/STRA = for | | | | | | | |
| 2 | 98910_at | Cluster Incl AI846118 UI-M-AK1-aew-e-07-0-UI.s1 Mus musculus cDNA, 3 end/clone = UI-M-AK1-aew-e-07-0-UI/ | 968.2 | P | 878.4 | A | 1600.5 | P | 1093.1 |
| | | clone_end = 3/gb = AI846118/gi = 5489989/ug = Mm.28594/len = 234/STRA = for | | | | | | | |
| 2 | 98916_at | Cluster Incl AI854289 UI-M-BH0-ajs-e-06-0-UI s1 Mus musculus cDNA, 3 end/clone = UI-M-BH0-ajs-e-06-0-UI/ | 205.4 | P | 203.6 | P | 227.2 | P | 184.9 |
| | | clone_end = 3/gb-AI854289/gi = 5498195/ug = Mm 28618/len = 490/STRA = for | | | | | | | |
| 2 | 96848_at | Cluster Incl AW120756 UI-M-BH2 3-any-d-12-0-UI s1 Mus musculus cDNA, 3 end/clone = UI-M-BH2 3-any-d-12-0-UI/ | 147.5 | P | 106.1 | P | 123.7 | P | 108.9 |
| | | clone_end = 3/gb = AW120756/gi = 6096089/ug = Mm 30030/len = 407/STRA = for | | | | | | | |
| 2 | 97489_at | Cluster Incl AI846739 UI-M-AO1-ael-b-05-0-UI s1 Mus musculus cDNA, 3 end/clone = UI-M-AO1-ael-b-05-0-UI/ | 434.4 | P | 438.5 | P | 533.6 | P | 340.8 |
| | | clone_end = 3/gb = AI846739/gi = 5490645/ug = Mm 30969/len = 279/STRA = for | | | | | | | |
| 2 | 98561_at | Cluster Incl AJ242874 Mus musculus mRNA for slow skeletal muscle troponin 1 (Tnnl1 gene)/cds = (143,706)/gb = AJ242674/ | 302.4 | P | 456.1 | P | 812.3 | P | 345.2 |
| | | gi = 5457404/ug = Mm 36900/len = 1054/STRA = for | | | | | | | |
| 2 | 100086_at | Cluster Incl D00622 Low density lipoprotein receptor related protein, associated protein 1/cds = (0,1081)/gb = D00622/ | 88.6 | P | 174.2 | P | 254.4 | P | 159.3 |
| | | gi = 220433/ug = Mm 4560/len = 2902/STRA = for | | | | | | | |
| 2 | 101583_at | Cluster Incl M64292B-cell translocation gene 2, anti-proliferative/cds = (86,542)/gb = M64292/gi = 202068/ug = Mm 903/ | 770.5 | P | 1054.7 | A | 1741.9 | P | 1170.1 |
| | | len = 2583/STRA = for | | | | | | | |
| 2 | 103423_at | Cluster Incl AI846517 UI-M-AN1-aff-e-02-0-UI s1 Mus musculus cDNA, 3 end/clone = UI-M-AN1-aff-e-02-0-UI/ | 327.9 | P | 438.6 | P | 395.9 | P | 244.8 |
| | | clone_end = 3/gb = AI846517/gi-5490423/ug = Mm 18942/len = 349/STRA = for | | | | | | | |
| 2 | 103622_at | Clusler Incl AW050255.UI-M-BH1-ans-b-11-0-UI s1 Mus musculus cDNA, 3 end/clone = UI-M-BH1-ans-b-11-0-UI/ | 319.7 | P | 382.5 | P | 335.9 | P | 352.6 |
| | | clone_end = 3/gb = AW050255/gi = 5910784/ug = Mm 20260/len = 397/STRA = for | | | | | | | |
| 2 | 103906_f_at | Cluster Incl AI840868 UI-M-AM0-adm-a-01-0-UI s1 Mus musculus cDNA. 3 end/clone = UI-M-AM0-adm-a-01-0-UI/ | 152.6 | P | 170.9 | P | 190.9 | P | 184.9 |
| | | clone_end = 3/gb = AI840868/gi = 5475081/ug = Mm 21401/len = 346/STRA = for | | | | | | | |
| 2 | 104701_at | Cluster Incl Y07836 M musculus mRNA for basic-helix-loop-helix protein/cds = (0,1214)/gb = Y07836/gi = 1707584/ | 198.3 | P | 187.8 | M | 609.1 | P | 427.4 |
| | | ug = Mm 2436/len = 1508/STRA = for | | | | | | | |
| 2 | 94978_at | Cluster Incl AIS89775 UI-M-AN0-acq-f-06-0-UI s1 Mus musculus cDNA, 3 end/clone = UI-M-AN0-acq-f-06-0-UI/ | 750.8 | A | 502.8 | P | 842.7 | P | 832.5 |
| | | clone_end = 3/gb = AI839775/gi = 5474028/ug = Mm 27269/len = 273/STRA = for | | | | | | | |
| 2 | 96227_at | Cluster Incl X70533 Corticosteroid binding globulin/cds = (54,1247)/gb = X70533/gi = 298114/ug = Mm 2874/len = 1462/ | 454 | P | 429.5 | P | 483.7 | P | 417.9 |
| | | STRA = for | | | | | | | |
| 2 | 96801_at | Cluster Incl AJ010108.Mus musculus mRNA for cytosolic adenylate Kinase/cds = (16,600)/gb = AJ010108/gi = 5725309/ | 88.2 | A | 289.4 | P | 261.8 | P | 140.3 |
| | | ug = Mm 29189/len = 1958/STRA = for | | | | | | | |
| 2 | 97960_at | Cluster Incl AW125800: UI-M-BH2.2-aql-h-05-0-UI.s1 Mus musculus cDNA, 3 end/clone = UI-M-BH2.2-aql-h-05-0-UI/ | 184.4 | P | 158.5 | P | 338.3 | P | 143.7 |
| | | clone_end = 3/gb = AW125800/gi = 6101330/ug = Mm.30602/len = 326/STRA = for | | | | | | | |
| 2 | 100952_at | Cluster Incl U47323: Stromal interaction molecule 1/cds = (193,2250)/gb = U47323/gi = 1493832/ug = Mm.645/len = 3192/ | 335.5 | P | 392 | P | 486.8 | P | 248 |
| | | STRA = for | | | | | | | |
| 2 | 101408_at | Cluster Incl AF010499: Guanldinoacetate methyltransferase/cds = (110,820)/gb = AF010499/gi = 2555082/ug = Mm 7329/ | 225 | P | 210.4 | P | 373.4 | P | 173.6 |
| | | len = 939/STRA = for | | | | | | | |
| 2 | 103731_at | Cluster Incl AI837319 UI-M-AK0-adf-c-02-0-UI.s1 Mus musculus cDNA, 3 end/clone = UI-M-AK0-adf-c-02-0-UI/ | 239.5 | P | 250.2 | P | 256.8 | P | 164.3 |
| | | clone end = 3/gb = AI837319/gi = 5471532/ug = Mm.24309/len = 418/STRA = rev | | | | | | | |
| 2 | 104156_r_at | Cluster Incl U19118 Mus musculus transcription factor LRG-21 mRNA, complete cds/cds = (167,712)/gb = U19118/ | 241.7 | P | 299.1 | P | 484.3 | P | 140.4 |
| | | gi = 633751/ug = Mm 2706/len = 1897/STRA = for | | | | | | | |
| 2 | 104262_at | Cluster Incl M60057: Parathyroid hormone-like peptide/cds = (25,552)/gb = M60057/gi = 200565/ug = Mm 28440/len = 1022/ | 500.4 | P | 382 | P | 526.4 | P | 370.8 |
| | | STRA = rev | | | | | | | |
| 2 | 92202_g_at | Cluster Incl AI553024: vf37g06 y1 Mus musculus cDNA, 5 end/clone = IMAGE-846010/clone_end = 5/gb = AI553024/ | 12.4 | A | 121.7 | P | 431.9 | P | 154.7 |
| | | gi = 4485387/ug = Mm 34106/len = 367/STRA = for | | | | | | | |
| 2 | 92235_g_at | Cluster Incl X66223: M musculus mRNA for retinoid X receptor-alpha (mRXR-alpha)/cds = (177,1580)/gb = X66223/ | 498.3 | P | 417 | A | 498.1 | P | 379.4 |
| | | gi = 54021/ug = Mm.3470/len = 2088/STRA = for | | | | | | | |
| 2 | 92382_at | Cluster Incl U49739 Myosin VI/cds = (159,3956)/gb = U49739/gi = 1322279/ug = Mm 4040/len = 4602/STRA = for | 9.2 | P | −100.4 | P | 4.2 | P | −66.8 |
| 2 | 92956_at | Cluster Incl X74760 Notch gene homolog 3, (Drosophila)/cds = (59,7015)/gb = X74760/gi = 483580/ug = Mm 4945/len = 7943/ | 450 | P | 453.6 | P | 550.4 | P | 2278.1 |
| | | STRA = for | | | | | | | |
| 2 | 93921_at | Cluster Incl AI573601 ud46a12 y1 Mus musculus cDNA, 5 end/clone = IMAGE-1448926/clone_end = 5/gb = AI573601/ | 383.8 | P | 366.1 | P | 247.4 | P | 498.4 |
| | | gi = 4536975/ug = Mm.89855/len = 535/STRA = for | | | | | | | |
| 2 | 93958_at | Cluster Incl AA086863 mm86g02 r1 Mus musculus cDNA, 5 end/clone = IMAGE-535346/clone_end = 5/gb = AA086863/ | 255.4 | P | 234.1 | P | 250.3 | P | 206.7 |
| | | gi = 1630072/ug = Mm.95627/len = 494/STRA = for | | | | | | | |
| 2 | 94097_at | Cluster Incl AB025922 Mus musculus Gli1 mRNA, complete cds/cds = (171,3506)/gb = AB025922/gi = 6009643/ | 318 | P | 336.2 | P | 371.4 | P | 267.2 |
| | | ug = Mm 10081/len = 3686/STRA = for | | | | | | | |
| 2 | 95889_at | Cluster Incl AI854770 UI-M-BH0-aka-c-09-0-UI s1 Mus musculus cDNA, 3 end/clone = UI-M-BH0-aka-c-09-0-UI/ | 279.3 | P | 367.4 | P | 451.7 | P | 306.7 |
| | | clone_end = 3/gb = AI854770/gi = 5498676/ug = Mm 24046/len = 417/STRA = for | | | | | | | |
| 2 | 97762_f_at | Cluster Incl U12890 Killer cell lectin-like receptor, subfamily A, member 7/cds = (21,824)/gb = U12890/gi = 602409/ | 201.2 | P | 179 | P | 206 | P | 152.3 |
| | | ug = Mm 4131/len = 921/STRA = for | | | | | | | |
| 2 | 98277_at | Cluster Incl AB017615: Mus musculus mRNA for Eos protein, complete cds/cds = (752,2353)/gb = AB017615/gi = 4062982/ | 472.8 | P | 473.9 | P | 583.2 | P | 365.9 |
| | | ug = Mm 42034/len = 2688/STRA = for | | | | | | | |
| 2 | 100289_at | Cluster Incl U92885: Mus musculus LERK-3 (Epl3) gene, partial cds/cds = (0,564)/gb = U92885/gi = 2843098/ug = Mm 8547/ | 914.5 | P | 712.9 | P | 1145.2 | P | 823.3 |
| | | len = 859/STRA = for | | | | | | | |
| 2 | 97652_at | Cluster Incl L07379 Growth hormone releasing hormone receptor/cds = UNKNOWN/gb = L07379/gi = 193510/ug = Mm 89928/ | 537.6 | P | 332.1 | P | 628.4 | P | 598.2 |
| | | len = 1320/STRA = for | | | | | | | |
| 2 | 101712_at | Cluster Incl AJ009823: Mus musculus mRNA for P2X7 receptor subunit/cds = (11,1798)/gb = AJ009323/gi = 4007637/ | 384.9 | P | 515.4 | A | 462.8 | P | 456.3 |
| | | ug = Mm.42026/len = 1810/STRA = for | | | | | | | |
| 2 | 102091_f_at | Cluster Incl AA407599.EST00874 Mus musculus cDNA, 3 end/clone = C0003E09/clone_end = 3/gb = AA407599/ | 92.2 | P | 88 | A | 105.6 | P | 93.6 |
| | | gi = 2065789/ug = Mm 42282/len = 285/STRA = rev | | | | | | | |
| 2 | 93799_at | Cluster Incl AI845933 UI-M-AK1-aex-f-02-0-UI.s1 Mus musculus cDNA, 3 end/clone = UI-M-AK1-aex-f-02-0-UI/ | 758 | P | 786.8 | P | 844.1 | A | 841 |
| | | clone_end = 3/gb = AI845933/gi = 5489839/ug = Mm 21407/len = 325/STRA = for | | | | | | | |
| 2 | 94208_at | Cluster Incl AW045202 UI-M-BH1-alh-c-08-0-UI.s1 Mus musculus cDNA, 3 end/clone = UI-M-BH1-alh-c-08-0-UI/ | 296.5 | P | 138.7 | A | 136.6 | P | 172.6 |
| | | clone_end = 3/gb = AW045202/gi = 5905731/ug = Mm.2220/len = 430/STRA = for | | | | | | | |
| 2 | 94274_at | Cluster Incl AI837415 UI-M-AP0-abm-f-06-0-UI s1 Mus musculus cDNA, 3 end/clone = UI-M-AP0-abm-f-06-0-UI/ | 504.4 | P | 232.2 | A | 737.6 | P | 274.7 |
| | | clone_end = 3/gb = AI837415/gi = 5471628/ug = Mm 22491/len = 360/STRA = for | | | | | | | |
| 2 | 102362_l_at | Cluster Incl U20735: Mus musculus transcription factor junB (junB) gene, 5 region and complete cds/cds = (294,1328)/ | 448.9 | P | 318.4 | A | 1257 | P | 826.9 |
| | | gb = U20735/gi = 965003/ug = Mm 1167/len = 1786/STRA = for | | | | | | | |
| 2 | 102371_at | Cluster Incl X16995 Hormone receptor/cds = (2,1807)/gb = X16995/gi = 53312/ug = Mm.119/len = 2351/STRA = for | 47.4 | P | −3 | A | 1542.7 | P | 130.3 |
| 2 | 104755_at | Cluster Incl AJ242778: Mus musculus mRNA for ABINI, (A20-binding inhibitor of NF-kappa B activation (large)/ | 326.2 | A | 533 | P | 659.5 | P | 292.3 |
| | | cds = (116,2059)/gb = AJ242778/gi = 4995752/ug = Mm 24782/len = 2812/STRA = rev | | | | | | | |
| 2 | 94336_at | Cluster Incl AI838318 UI-M-AO0-aby-f-01-0-UI s1 Mus musculus cDNA, 3 end/clone = UI-M-AO0-aby-f-01-0-UI/ | 392.2 | P | 534.7 | P | 420 | P | 375.6 |
| | | clone_end = 3/gb = AI838318/gi = 5472531/ug = Mm 2498/len = 186/STRA = rev | | | | | | | |
| 2 | 99991_at | Cluster Incl U31993.lnterleukin 17 receptor/cds = (120,2714)/gb = U31993/gi = 1161342/ug = Mm 4481/len = 3288/STRA = for | 133.3 | P | 119.9 | A | 185.9 | P | 127.4 |
| 2 | 103204_r_at | Cluster Incl AW259499 up28d02 x1 Mus musculus cDNA, 3 end/clone = IMAGE-2655651/clone_end = 3/gb = AW259499/ | 268 | P | 159.6 | P | 181.3 | P | 135.1 |
| | | gi = 6632417/ug = Mm.1911/len = 454/STRA = for | | | | | | | |
| 2 | 103551_at | Cluster Incl AW124208 UI-M-BH2 1-aph-c-03-0-UI.s1 Mus musculus cDNA, 3 end/clone = UI-M-BH2.1-aph-c-03-0-UI/ | 84.4 | A | 26.2 | A | 135.1 | P | 87.4 |
| | | clone_end = 3/gb = AW124208/gi = 6099738/ug = Mm 23813/len = 384/STRA = for | | | | | | | |
| 2 | 92500_at | Cluster Incl AB025412 Mus musculus mRNA for Ten-m3, complete cds/cds = (49,8196)/gb = AB025412/gi = 4760779/ | 22.1 | P | 25.6 | P | 1.6 | P | 4.2 |
| | | ug = Mm 42191/len = 8964/STRA = for | | | | | | | |
| 2 | 92923_g_at | Cluster Incl U17291: Mus musculus transcription factor AP-2 (AP-2) gene, alternative/cds = (36,602)/gb = U17291/ | 200.2 | P | 134.7 | P | 316.6 | P | 231.7 |
| | | gi = 755199/ug = Mm 4845/len = 603/STRA = for | | | | | | | |
| 2 | 93718_at | Cluster Incl Z22821 Mus musculus mRNA for Rab23 protein/cds = (20,733)/gb = Z22821/gi = 438161/ug = Mm 86744/ | 85.8 | P | 86.2 | P | 59 | P | 57.3 |
| | | len = 740/STRA = for | | | | | | | |
| 2 | 93724_at | Cluster Incl AI596034 uk22a01.x1 Mus musculus cDNA, 3 end/clone = IMAGE-1969704/clone_end = 3/gb = AI596034/ | 169.4 | P | 156.6 | P | 195.5 | P | 157.8 |
| | | gi = 4605082/ug = Mm.86922/len = 516/STRA = rev | | | | | | | |
| 2 | 94133_r _at | Cluster Incl U94423: Mus musculus MEF2A mRNA, partial 3 UTR sequence/cds = UNKNOWN/gb = U94423/gi = 2088559/ | 49.6 | P | 27.2 | A | 45.8 | P | 39.4 |
| | | ug = Mm 11087/len = 1016/STRA = rev | | | | | | | |
| 2 | 95360_at | Cluster Incl AA259683: va36d09 r1 Mus musculus cDNA, 5 end/clone = IMAGE-733457/clone_end = 5/gb = AA259683/ | 39.6 | A | 10.4 | A | 6.2 | P | 19 |
| | | gi = 1896186/ug = Mm.21902/len = 349/STRA = for | | | | | | | |
| 2 | 97171_f_at | Cluster Incl AI060627 Ub42b11.r1 Mus musculus cDNA, 5 end/clone = IMAGE-1380381/clone_end = 5/gb = AI060627/ | 159.3 | P | 248.9 | P | 338.3 | P | 334.2 |
| | | gi = 3336050/ug = Mm 35621/len = 335/STRA = for | | | | | | | |
| 2 | 98291_at | Cluster Incl AF092050: Mus musculus beta-1,3-N-acetylglucosaminyltransferase mRNA, complete cds/cds = (4,981)/ | 362.6 | P | 230.2 | P | 320.2 | P | 205.2 |
| | | gb = AF092050/gi = 4191391/ug = Mm.42092/len = 1536/STRA = for | | | | | | | |
| 2 | 101769_at | Cluster Incl AB008178: Mus musculus CNR3 mRNA for Cadherin-related neural recepter 3, partial cds/cds = (0,2730)/ | 1036.2 | P | 860.1 | P | 1215.4 | P | 1030.4 |
| | | gb = AB008178/gi = 3253078/ug = Mm.57101/len = 2731/STRA = for | | | | | | | |
| 2 | 101603_g_at | Cluster Incl C79108: C79108 Mus musculus cDNA, 3 end/clone = J0060E04/clone_end = 3/gb = C79108/gi = 2519438/ | 238 8 | P | 226.4 | P | 122.8 | A | 157.6 |
| | | ug = Mm 5769/len = 579/STRA = rev | | | | | | | |
| 2 | 92768_s_at | Cluster Incl M15268.Aminolevulinic acid synthase 2, erythroid/cds = (0,1760)/gb = M15268/gi = 191857/ug = Mm 1217/ | 103.1 | A | 660.8 | P | 699.3 | P | 109.8 |
| | | len = 1896/STRA = for | | | | | | | |
| 2 | 93073_at | Cluster Incl U36576 Nuclear factor of activated T-cells, cytoplasmic 2/cds = (70,2853)/gb = U36576/gi = 1353400 | 1218.7 | P | 851.7 | A | 1180.7 | P | 819.9 |
| | | ug = Mm 1574/len = 3518/STRA = rev | | | | | | | |
| 2 | 93583_s_at | Cluster Incl V00817: Mouse germ line gene fragment for mu-immunoglobulin C-terminus (secreted form)/cds = (0,83)/ | 391.9 | P | 496.8 | P | 330 | P | 321 |
| | | gb = V00817/gi = 52345/ug = Mm 2066/len = 210/STRA = for | | | | | | | |
| 2 | 94046_at | Cluster Incl U05247 C-src tyrosine kinase/cds = (0,1352)/gb = U05247/gi = 452471/ug = Mm.21974/len = 1779/STRA = for | 571 | P | 582 | M | 566.1 | M | 472.4 |
| 2 | 94856_r_at | Cluster Incl AI842362 UI-M-AM1-agc-a-03-0-UI s1 Mus musculus cDNA, 3 end/clone = UI-M-AM1-agc-a-03-0-UI/ | 90.4 | P | 100.5 | P | 92.1 | P | 74.1 |
| | | clone_end = 3/gb = AI842362/gi = 5476575/ug = Mm 23566/len = 298/STRA = for | | | | | | | |
| 2 | 96026_at | Cluster Incl AA987153: uc80h10.x1 Mus musculus cDNA, 3 end/clone = IMAGE-1432003/clone_end = 3/gb = AA987153/ | 114.5 | P | 108.8 | P | 105.9 | A | 126.4 |
| | | gi = 3167916/ug = Mm.2573/len = 824/STRA = for | | | | | | | |
| 2 | 97906_at | Cluster Incl Z19581.Seven in absentia 2/cds = (289,1266)/gb = Z19581/gi = 297036/ug = Mm 2847/len = 2198/STRA = for | 555.2 | P | 376.8 | A | 530.3 | P | 522.2 |
| 2 | 99186_at | Cluster Incl X75483 Cyclin A2/cds = (121,1389)/gb = X75483/gi = 414061/ug = Mm.4189/len = 1662/STRA = for | 218.6 | P | 405.6 | P | 313 | P | −78.1 |
| 2 | 101063_at | Cluster Incl M29793: M musculus slow/cardiac troponin C (cTnC) mRNA, complete cds/cds = (43,528)/gb = M29793/ | 7.8 | A | 396.6 | P | 310.4 | P | 232.7 |
| | | gi = 192839/ug = Mm 712/len = 703/STRA = for | | | | | | | |
| 2 | 101505_at | Cluster Incl AW121234 UI-M-BH2 3-aok-f-04-0-UI s1 Mus musculus cDNA, 3 end/clone = UI-M-BH2 3-aok-f-04-0-UI/ | 142.4 | P | 142.4 | P | 133.3 | P | 154.7 |
| | | clone_end = 3/gb = AW121234/gi = 6096567/ug = Mm 8195/len = 426/STRA = for | | | | | | | |
| 2 | 94947_g_at | Cluster Incl U43187: Mus musculus MEK kinase 3, mRNA, partial cds/cds = (331,2211)/gb = U43187/gi = 1223901/ | 70.8 | A | 79.8 | A | 101.8 | P | 96.2 |
| | | ug = Mm 27041/len = 3332/STRA = for | | | | | | | |
| 2 | 97358_at | Cluster Incl AI851356 UI-M-BH0-akh-d-11-0-UI.s1 Mus musculus cDNA, 3 end/clone = UI-M-BH0-akh-d-11-0-UI/ | 194 | M | 546.2 | A | 363.7 | P | 274.8 |
| | | clone_end = 3/gb = AI851356/gi = 5495262/ug = Mm.29638/len = 401/STRA = for | | | | | | | |
| 2 | 97942_g_at | Cluster Incl Y12582 M musculus mRNA for calpain-like protease/cds = (129,2054)/gb = Y12582/gi = 2239139/ug = .Mm 30290/ | 122.4 | A | 256.4 | P | 489.7 | P | 153.6 |
| | | len = 2069/STRA = for | | | | | | | |
| 2 | 100432_f_at | Cluster Incl U57820 MyoD family inhibitor/cds = (30,770)/gb = U57820/gi = 1545983/ug = Mm 4763/len = 1360/STRA = for | 1138.6 | P | 928.5 | P | 1363.8 | P | 1185.2 |
| 2 | 93669_f_at | Cluster lncl AW107922 um24d07 x1 Mus musculus cDNA, 3 end/clone = IMAGE-2225485/clone_end = 3/gb = AW107922/ | 16 | P | 38.1 | P | 10.3 | A | 26.5 |
| | | gi = 6078722/ug = Mm 80595/len = 576/STRA = rev | | | | | | | |
| 2 | 94770_at | Cluster Incl AI553596 vw47f05.x1 Mus musculus cDNA, 3 end/clone = IMAGE-1246977/clone_end = 3/gb = AI553596/ | 233.9 | A | 244.2 | P | 307 | P | 279.7 |
| | | gi = 4485959/ug = Mm 1648/len = 341/STRA = for | | | | | | | |
| 2 | 95340_at | Cluster Incl M93310 Mouse metailothionein-III gene, complete cds/cds = (56,262)/gb = M93310/gi = 199133/ug = Mm 2064/ | 962 | M | 1169 | M | 1075.3 | P | 1015.6 |
| | | len = 401/STRA = for | | | | | | | |
| 2 | 97769_at | Cluster Incl D17433 Prostaglandin F receptor/cds = (223,1323)/gb = D17433/gi = 457656/ug = Mm.4170/len = 2170/ | 160 | P | 180.8 | P | 155.9 | A | 183 |
| | | STRA = rev | | | | | | | |
| 2 | 100736_at | Cluster Incl L77900 Mus musculus Barx2 mRNA, complete cds/cds = (67,753)/gb = L77900/gi = 2055363/ug = Mm 3508/ | 95 | P | 85.2 | P | 69.1 | P | 102.3 |
| | | len = 1274/STRA = for | | | | | | | |
| 2 | 92619_at | Cluster Incl U40825 Mus musculus WW-domain binding protein 1 mRNA, complete cds/cds = (152,1069)/gb = U40825/ | 1106.3 | P | 1188.4 | P | 1313 | P | 1081.2 |
| | | gi = 1777576/ug = Mm 1109/len = 1292/STRA = for | | | | | | | |
| 2 | 92820_at | Cluster Incl AI846522 UI-M-AN1-aff-e-09-0-UI.s1 Mus musculus cDNA, 3 end/clone = UI-M-AN1-aff-e-09-0-UI/ | 259.1 | A | 237.3 | A | 579.6 | P | 381.6 |
| | | clone_end = 3/gb = AI846522/gi = 5490428/ug = Mm 12914/len = 370/STRA = rev | | | | | | | |
| 2 | 93507_at | Cluster Incl X62622 Tissue inhibitor of metalloproteinase 2/cds = (226,888)/gb = X62622/gi = 54801/ug = Mm 19191/ | 292.6 | P | 268.2 | P | 276.1 | P | 193.2 |
| | | len = 1714/STRA = for | | | | | | | |
| 2 | 94815_at | Cluster Incl X13586 Murine mRNA for 2,3-bisphosphoglycerate mutase (BPGM; EC 5.4,2.4)/cds = (20,799)/gb = X13586/ | 187.1 | A | 298.9 | P | 263 | P | 106 |
| | | gi = 50183/ug = Mm 22706/len = 825/STRA = for | | | | | | | |
| 2 | 94906_at | Cluster Incl M22679 Mouse alcohol dehydrogenase class I gene (ADH-A-2)/cds = (0,1127)/gb = M22679/gi = 191715/ | 230.8 | A | 412 | P | 1166.3 | P | 73.8 |
| | | ug = Mm.2409/len = 1128/STRA = for | | | | | | | |
| 2 | 101046_at | Cluster Incl X56397: Mouse mRNA for vimentin/cds = (84,1484)/gb = X56397/gi = 55407/ug = Mm 7/len = 1748/STRA = for | 300.4 | P | 300.5 | P | 290.2 | P | 260.7 |
| 2 | 101531_at | Cluster Incl AI527354.uj51b11.x1 Mus musculus cDNA, 3 end/clone = IMAGE-1923453/clone_end = 3/gb = AI527354/ | 237.4 | A | 766.2 | P | 923.8 | P | 288.2 |
| | | gi = 4441489/ug = Mm 87581/len = 534/STRA = rev | | | | | | | |
| 2 | 102429_at | Cluster Incl AB005451: Mus musculus mRNA for RST, complete cds/cds = (99,1760)/gb = AB005451/gi = 2696708/ | 497 | P | 665.5 | P | 505.2 | A | 400.8 |
| | | ug = Mm.12846/len = 1779/STRA = for | | | | | | | |
| 2 | 104047_at | Cluster Incl AW122136: UI-M-BH2.2-aor-d-08-0-UI s1 Mus musculus cDNA, 3 end/clone = UI-M-BH2 2-aor-d-08-0-UI/ | 147.8 | P | 151.5 | P | 350.8 | A | 240.7 |
| | | clone end = 3/gb = AW122136/gi = 6097563/ug = Mm 21495/len = 276/STRA = for | | | | | | | |
| 2 | 99021_at | Cluster Incl U03873 Paired masoderm homeobox 1/cds = (1021,1674)/gb = U03873/gi = 460124/ug = Mm 3869/len = 3359/ | 83.4 | A | 64.9 | P | 69.1 | A | 71.4 |
| | | STRA = for | | | | | | | |
| 2 | 99055_at | Cluster Incl X94000: M. musculus Gal beta-1,3-GaINAc-specific GaINAc alpha-2,6-aiatyltransferase gene (1804 bp)/ | 769.6 | P | 755.8 | P | 735.6 | P | 674 |
| | | cds = (67,153)/gb-X94000/gi = 1526610/ug = Mm.3947/len = 154/STRA = for | | | | | | | |
| 2 | 100449_g_at | Cluster Incl Z31664 M musculus (NIH Swiss) mRNA for ALK-1/cds = (240,1748)/gb = Z31684/gi = 840814/ug = Mm 4839/ | 362.9 | A | 413 | M | 240.7 | P | 189.9 |
| | | len = 1909/STRA = for | | | | | | | |
| 2 | 103211_at | Cluster Incl AI852574: UI-M-BH0-aiu-a-02-0-UI s1 Mus musculus cDNA, 3 end/clone = UI-M-BH0-alu-a-02-0-UI/ | 850.7 | A | 980.4 | A | 912.5 | P | 848.6 |
| | | clone_end = 3/gb = AI852574/gi = 5496480/ug = Mm 1960/len = 235/STRA = rev | | | | | | | |
| 2 | 103789_at | Cluster Incl AI854614: UI-M-BH0-ake-d-06-0-UI s1 Mus musculus cDNA, 3 end/clone = UI-M-BH0-ake-d-06-0-UI/ | −4.8 | P | 10.9 | A | 80.8 | P | 57.6 |
| | | clone_end = 3/gb = AI854614/gi = 5498520/ug = Mm 24948/len = 446/STRA = for | | | | | | | |
| 2 | 92532_at | Cluster Incl D49730: Mouse mRNA for V1a agrinine vasopression receptor, complete cds/cds = (312,1583)/gb = D49730/ | 228.8 | A | 252.5 | M | 322.1 | P | 292.3 |
| | | gi = 1722700/ug = Mm.4351/len = 2684/STRA = rev | | | | | | | |
| 2 | 93870_at | Cluster Incl M64429 Braf transforming gene/cds = (37,2016)/gb = M64429/gi = 553877/ug = Mm 8807/len = 2050/STRA = for | 116.3 | P | 70.2 | A | 83 | A | 51.9 |
| 2 | 94142_at | Cluster Incl M13926 Colony stimulating factor, granulocyte/cds = (87,693)/gb = M13926/gi = 193451/ug = Mm 1238/len = 1363/ | 128.4 | A | 430.9 | A | 970.6 | P | 196.6 |
| | | STRA = for | | | | | | | |
| 2 | 94784_at | Cluster Incl AI593484 vu42d01 x1 Mus musculus cDNA, 3 end/clone = IMAGE-1194049/clone_end = 3/gb = AI593484/ | 232.6 | P | 154.3 | P | 315.4 | P | 107.1 |
| | | gi = 4602532/ug = Mm.1689/len = 477/STRA = for | | | | | | | |
| 2 | 95881_f_at | Cluster Incl AI447783 mq84c02.x1 Mus musculus cDNA, 3 end/clone = IMAGE-585410/clone_end = 3/gb = AI447783/ | 315.5 | P | 322.7 | P | 317.3 | M | 342 |
| | | gi = 4306405/ug = Mm.23936/len = 347/STRA = rev | | | | | | | |
| 2 | 96574_at | Cluster Incl M30136 Interleukin 9/cds = (0,434)/gb = M30136/gi = 200190/ug = Mm 3006/len = 435/STRA = for | 301.6 | P | 212.3 | A | 327.2 | P | 236.5 |
| 2 | 97147_at | Cluster Incl C80892: C80892 Mus musculus cDNA, 3 end/clone = J0090C03/clone_end = 3/gb = C80892/gi = 2521222/ | 35.1 | P | 34.7 | P | 68.8 | P | 15 |
| | | ug = Mm 34947/len = 585/STRA = rev | | | | | | | |
| 2 | 100291_at | Cluster Incl X57111.Casitas B-lineage lymphoma/cds = (96,2786)/gb = X57111/gi = 50314/ug = Mm.8585/len = 2808/ | 34.1 | M | 55.6 | A | 77 | A | 35.7 |
| | | STRA = for | | | | | | | |
| 2 | 94270_at | Cluster Incl M22832 Mus musculus cytokeratin (endoB) gene, complete cds/cds = (58,1329)/gb = M22832/gi = 340757/ | 137.3 | A | 150.8 | A | 185.7 | P | 132.7 |
| | | ug = Mm 22479/len = 1373/STRA = for | | | | | | | |
| 2 | 94308_at | Cluster Incl X70853.BM-90/fibulin extracellular matrix glyroprotein/cds = (0,353)/gb = X70853/gi = 396816/ug = Mm 22626/ | 392.2 | M | 360.3 | M | 494.4 | P | 360.6 |
| | | len = 448/STRA = for | | | | | | | |
| 2 | 100130_at | Cluster Incl X12761: Jun oncogene/cds = (354,1358)/gb = X12761/gi = 52758/ug = Mm 482/len = 2565/STRA = for | 208 | A | 391.6 | A | 208 | P | 147.8 |
| 2 | 102071_at | Cluster Incl AIB51565: UI-M-BHO-aki-f-01-0-UI s1 Mus musculus cDNA, 3 end/clone = UI-M-BH0-aki-f-01-0-UI/clone_end = 3/ | 487.2 | A | 500.5 | M | 480.5 | P | 532.4 |
| | | gb = AI851565/gi = 5495471/ug = Mm 10641/len = 341/STRA = for | | | | | | | |
| 2 | 103088_at | Cluster Incl X94310: M musculus mRNA for L1-like protein/cds = (295,3924)/gb = X94310/gi = 1532034/ug = Mm 1736/ | 101.2 | A | 90.4 | P | 95.5 | M | 68.6 |
| | | len = 4437/STRA = for | | | | | | | |
| 2 | 103420_at | Cluster Incl U79753 Emerin/cds = (106,885)/gb = U79753/gi = 1916802/ug = Mm 18892/len = 1270/STRA = for | 667.5 | A | 846.7 | P | 914.7 | A | 809.3 |
| 2 | 104393_at | Cluster Incl AI843571 UI-M-AO1-aen-d-09-0-UI.s1 Mus musculus cDNA, 3 end/clone = UI-M-AO1-aen-d-09-0-UI/ | −82.1 | A | 0.2 | P | 23.3 | P | −22.6 |
| | | clone_end = 3/gb = AI843571/gl = 5477824/ug = Mm 2275/len = 290/STRA = for | | | | | | | |
| 2 | 95566_at | Cluster Incl AF050666: Glycosylphosphatidytinositol specific phospholipase D1/cds = (270,2783)/gb = AF050666/ | 347.7 | P | 384.6 | A | 426 | P | 235.1 |
| | | gi = 2984690/ug = Mm.2779/len = 3436/STHA = for | | | | | | | |
| 2 | 100015_at | Cluster Incl X67677 Yamaguchi sarcoma viral (v-yes) oncogene homolog/cds = (602,2227)/gb = X67677/gi = 50623/ | 487.4 | P | 278.6 | P | 219.3 | A | 302.1 |
| | | ug = Mm 4558/len = 4550/STRA = for | | | | | | | |
| 2 | 102202_s_at | Cluster Incl M36411 Mpv17 transgene, kidney disease mutant/cds = (48,578)/gb = M36411/gi = 199789/ug = Mm 999/ | 157 | A | 147.5 | A | 186.4 | A | 168.4 |
| | | len = 1411/STRA = rev | | | | | | | |
| 2 | 103214_at | Cluster Incl AW048883 UI-M-BH1-amf-f-01-0-UI s1 Mus musculus cDNA, 3 end/clone = UI-M-BH1-amf-f-01-0-UI/ | 43.1 | A | 40.2 | A | 120.1 | P | 100.6 |
| | | clone_end = 3/gb = AW048883/gi = 5909412/ug = Mm 1980/len = 489/STRA = for | | | | | | | |
| 2 | 104455_at | Cluster Incl J03057 Mouse brain Ca++/calmodulin-dependant protein kinase type IV (CAM kinase IV) mRNA, 3 end/ | 64.1 | P | 70 | M | 88.8 | A | 110.9 |
| | | cds = (0,667)/gb = J03057/gi = 192366/ug = Mm 2951/len = 773/STRA = for | | | | | | | |
| 2 | 93619_at | Cluster Incl AF022992 Period homolog (Drosophila)/cds = (225,4100)/gb = AF022992/gi = 2435508/ug = Mm 7373/len = 4701/ | 22.8 | A | 149.1 | A | 522.3 | P | 116.2 |
| | | STRA = for | | | | | | | |
| 2 | 94748_g_at | Cluster Incl M34397.Interieukin 3 receptor, beta chain 1/cds = (287,2977)/gb = M34397/gi = 191821/ug = Mm 14595/ | 157 | M | 208.3 | A | 208.7 | P | 276.8 |
| | | len = 3113/STRA = for | | | | | | | |
| 2 | 96592_at | Cluster Incl U50413 Phosphatidylinositol 3-kinase, regulatory subunit, polypeptide 1 (p85 alpha)/cds = (574,2748)/ | 50.2 | P | 45.8 | A | 88.6 | A | 78.2 |
| | | gb = U50413/gi = 1621039/ug = Mm.3058/len = 3454/STRA = rev | | | | | | | |
| 2 | 97169_f_at | Cluster Incl AF067058 Mus musculus clone L10 variable group of 2-cell-stage gene family mRNA, complete cds/ | 124.5 | A | 33.6 | P | 109.2 | P | 86 |
| | | cds = (166,675)/gb = AF067058/gi = 3171133/ug = Mm 35599/len = 874/STRA = for | | | | | | | |
| 2 | 98840_at | Cluster Incl AF012104 Mus musculus cytoplasmic tyrosine kinase (bmx) mRNA, complete cds/cds = (8,1963)/ | 18.9 | P | 38.7 | A | 96.8 | M | 44.3 |
| | | gb = AF012104/gi = 2293557/ug = Mm 504/len = 2369/STRA = for | | | | | | | |
| 2 | 100280_at | Cluster Incl X72805 M musculus gene for testicular hislone H1/cds = (0,626)/gb = X72805/gi = 1934959/ug = Mm 8048/ | 231.7 | A | 272.9 | P | 244.2 | P | 217.2 |
| | | len = 627/STRA = for | | | | | | | |
| 2 | 100742_at | Cluster Incl AA409749 EST01534 Mus musculus cDNA, 3 end/clone = C0011A07/clone_end = 3/gb = AA409749/ | 76.6 | A | 185.5 | A | 203.6 | P | 175.1 |
| | | gi = 2066449/ug = Mm.3628/len = 337/STRA = rev | | | | | | | |
| 2 | 101168_at | Cluster Incl Z31360.M. musculus (Balb/C) P/L01 mRNA/cds = (0,139)/gb = Z31360/gi = 467579/ug = Mm 5029/len = 421/ | 90.2 | M | 30.4 | A | 64.1 | P | 52.4 |
| | | STRA = for | | | | | | | |
| 2 | 101185_at | Cluster Incl X92498 Forkhead homolog 6, (Drosophila)/cds = (277,1290)/gb = X92498/gi = 1199829/ug = Mm.5111/len = 1761/ | 397.2 | A | 478.7 | P | 473.3 | P | 255.7 |
| | | STRA = for | | | | | | | |
| 2 | 99585_at | Cluster Incl AI894122: md85f04 x1 Mus musculus cDNA, 3 end/clone = IMAGE-375199/clone_end = 3/gb = AI894122/ | 503.7 | P | 453.8 | M | 633.2 | P | 400.2 |
| | | gi = 5600024/ug = Mm 4261/len = 425/STRA = for | | | | | | | |
| 2 | 102401 _at | Cluster Incl M21085 Interferon regulatory factor 1/cds = (205,1194)/gb = M21065/gi = 198458/ug = Mm.1246/len = 2072/ | 646.4 | A | 335.9 | A | 1017.1 | P | 712.3 |
| | | STRA = for | | | | | | | |
| 2 | 103084_at | Cluster Incl D88791 Mouse mRNA for muscle LIM protein, complete cds/cds = (0,584)/gb = D88791/gi = 1695699/ | 25.9 | A | 57.7 | A | 370.7 | A | 108.1 |
| | | ug = Mm 17235/len = 585/STRA = for | | | | | | | |
| 2 | 103573_at | Cluster Incl D86176.Mouse mRNA for phosphatidylinositol 4-phosphate 5-kinase type I-alpha, complete cds/cds = (84,1703)/ | 62.4 | A | 87.1 | P | 101.5 | P | 79.3 |
| | | gb = D86176/gi = 1622707/ug = Mm 1945/len = 2378/STRA = for | | | | | | | |
| 2 | 103854_at | Cluster Incl AW049387 UI-M-BH1-ane-d-08-0-UI s1 Mus musculus cDNA, 3 end/clone = UI-M-BH1-ane-08-0-UI/ | 98.7 | M | 44.5 | P | 88.4 | M | 76.9 |
| | | clone_end = 3/gb = AW049387/gi = 5909916/ug = Mm.21071/len = 335/STRA = for | | | | | | | |
| 2 | 104342_i_at | Cluster Incl AI845798.UI-M-AQ1-aeb-h-07-0-UI s1 Mus musculus cDNA, 3 end/clone = UI-M-AQ1-aab-h-07-0-UI/ | −62.8 | A | −97.3 | P | −17.3 | A | −6.8 |
| | | clone_end = 3/gb = AI845798/gi = 5489704/ug = Mm.22526/len = 184/STRA = for | | | | | | | |
| 2 | 96160_at | Cluster Incl X67056: Glycine transporter 1/cds = (240,2141)/gb = X67058/gi = 51092/ug = Mm 2834/len = 3227/STRA = for | 562.4 | P | 427.3 | M | 408.3 | A | 411.6 |
| 2 | 96702_at | Cluster Incl AI787317: uj66c05 x1 Mus musculus cDNA, 3 end/clone = IMAGE-1924804/clone_end = 3/gb = AI787317/ | 94.2 | P | 87.8 | P | 84.4 | A | 62.5 |
| | | gi = 5885993/ug = Mm 20123/len = 470/STRA = for | | | | | | | |
| 2 | 92205_at | Cluster Incl AF090738: Mus musculus insulin receptor substrate-2 (Irs2) gene, partial cds/cds = (0,2206)/gb = AF090738/ | 178.6 | A | 127.4 | A | 367.5 | P | 251.9 |
| | | gi = 3661524/ug = Mm.34143/len = 2207/STRA = for | | | | | | | |
| 2 | 92254_at | Cluster Incl M55253: Myosin Vb/cds = (46.2217)/gb = M55253/gi = 193414/ug = Mm 3536/len = 2632/STRA = for | −200.2 | A | −102.1 | A | −7.6 | M | −37.2 |
| 2 | 98290_at | Cluster Incl AF027185 Mus musculus ELP mRNA, complete cds/cds = (35,430)/gb = AF027185/gi = 4103746/ug = Mm 42090/ | 362.1 | M | 400.9 | M | 415.3 | A | 447.1 |
| | | len = 1081/STRA = for | | | | | | | |
| 2 | 98770_at | Cluster Incl AF012708: Mus musculus centromere protein C (Cenp-c) gene, promoter region and partial cds/cds = (142,159)/ | 71.6 | P | 53.8 | A | 33.3 | A | 83.7 |
| | | gb = AF012708/gi = 2465200/ug = Mm 4649/len = 160/STRA = for | | | | | | | |
| 2 | 101225_at | Cluster Incl C77257 C77257: Mus musculus cDNA, 3 end/clone = J0028804/clone_end = 3/gb = C77257/gi = 2517587/ | 222.9 | M | 320.2 | M | 358.4 | M | 324.2 |
| | | ug = Mm 5593/len = 501/STRA = rev | | | | | | | |
| 3 | 92575_at | Cluster Incl U85489: Mus musculus Ah receptor-interacting protein (AIP) mRNA, complete cds/cds = (209,1201)/gb = U85489/ | 580.2 | P | 496.5 | P | 631.9 | P | 501 |
| | | gi = 2177173/ug = Mm 10433/len = 1367/STRA = for | | | | | | | |
| 3 | 92635_at | Cluster Incl M13444 Mouse alpha-tublin isotype M-alpha-4 mRNA, complete cds/cds = (29,1375)/gb = M13444/gi = 202214/ | 586 | P | 712.3 | P | 593.7 | P | 544.3 |
| | | ug = Mm 1155/len = 1442/STRA = for | | | | | | | |
| 3 | 92642_at | Cluster Incl M25944 Mouse carbonic anhydrase II (CAII) mRNA, 3 end/cds = (0,62)/gb = M25944/gi = 199078/ug = Mm 1186/ | 165.6 | P | 145.9 | P | 237.9 | P | 101.9 |
| | | len = 742/STRA = for | | | | | | | |
| 3 | 92778_i_at | Cluster Incl Z22552: M musculus membrane glycoprotein gene/cds = (0,223)/gb = Z22552/gi = 296166/ug = Mm. 1232 | 801.6 | P | 942.9 | P | 934.1 | P | 828.1 |
| | | len = 224/STRA = for | | | | | | | |
| 3 | 92821_at | Cluster Incl AF079565: Mus musculus ubiquitin-specific protease UBP41 (Uop41) mRNA, complete cds/cds = (0,1061)/ | 204.4 | P | 83.1 | P | 399.9 | P | 245.5 |
| | | gb = AF079565/gi = 3386551/ug = Mm 12914/len = 1115/STRA = for | | | | | | | |
| 3 | 92836_at | Cluster Incl AA919594 vz22b07 r1 Mus musculus cDNA, 5 end/clone = IMAGE-1316437/clone_end = 5/gb = AA919594/ | 2567.7 | P | 3135 | P | 3242.2 | P | 2991 |
| | | gi = 3066373/ug = Mm. 13097/len = 222/STRA = for | | | | | | | |
| 3 | 92852_at | Cluster Incl M18194 Mouse fibronectin (FN) mRNA/cds = (0,311)/gb = M18194/gi = 193329/ug = Mm 13846/len = 905/ | 1798.9 | P | 2199 | P | 2138.7 | P | 2108.4 |
| | | STRA = for | | | | | | | |
| 3 | 92863_at | Cluster Incl U40826 Mus musculus WW-domain binding protein 2 mRNA, complete cds/cds = (39,824)/gb = U40826/ | 206.5 | P | 422.2 | P | 404.9 | P | 560.1 |
| | | gi = 1777578/ug = Mm 1405/len = 1802/STRA = for | | | | | | | |
| 3 | 92885_at | Cluster Incl L48989: Troponin T3, skeletal, fast/cds = (0,719)/gb = L48989/gi = 2340049/ug = Mm 14546/len = 720/STRA = for | 2596.9 | P | 4031.6 | P | 3387.9 | P | 3586.8 |
| 3 | 93011_at | Cluster Incl AW123904 UI-M-BH2.1-app-g-10-0-UI s1 Mus musculus cDNA, 3 end/clone = UI-M-BH2.1-app-g-10-0-UI/ | 584.3 | P | 693.7 | P | 758.8 | P | 657.2 |
| | | clone_end = 3/gb = AW123904/gi = 6099434/ug = Mm.14638/len = 433/STRA = for | | | | | | | |
| 3 | 93048_at | Cluster Incl AJ005253 Mus musculus mRNA for CipP protease/cds = (32,850)/gb = AJ005253/gi = 3559934/ug = Mm.15243/ | 296.2 | P | 492.5 | P | 306.6 | P | 382.9 |
| | | len = 990/STRA = for | | | | | | | |
| 3 | 93053_at | Cluster Incl AF068244 Mus musculus cardiac calsequesirin mRNA, complete cds/cds = (129,1376)/gb = AF068244/ | 305.9 | P | 561.3 | P | 463 | P | 459.8 |
| | | gi = 3219362/ug = Mm.15343/len = 2275/STRA = for | | | | | | | |
| 3 | 93063_at | Cluster Incl U82624 Mus musculus amyloid precursor protein (APP) gene, partial cds, exons 16 and 17/cds = (0,247)/ | 2816 | P | 2859.5 | P | 2877 | P | 2665.5 |
| | | gb = U82624/gi = 1778801/ug = Mm 15571/len = 248/STRA = for | | | | | | | |
| 3 | 93084_at | Cluster Incl U27315 Adenine nucleotide translocator 1, skeletal muscle/cds = (86,982)/gb = U27315/gi = 902007/ | 2262.3 | P | 2968.6 | P | 2196.1 | P | 2446.9 |
| | | ug = Mm.16228/len = 1273/STRA = for | | | | | | | |
| 3 | 93086_at | Cluster Incl M18237 Mouse lg kappa chain V-region mRNA, partial cds, clone 3H9/cds = (0,321)/gb = M18237/gi = 197007/ | 192 | P | 294.8 | P | 286.8 | P | 200.3 |
| | | ug = Mm 16253/len = 322/STRA = for | | | | | | | |
| 3 | 93284_at | Cluster Incl D78135 Mus musculus mRNA for CIRP, complete cds/cds = (82,600)/gb = D78135/gi = 1060918/ug = Mm 17898/ | 637.2 | P | 1150.7 | P | 1272.7 | P | 849 |
| | | len = 1256/STRA = for | | | | | | | |
| 3 | 93330_at | Cluster Incl L02914 Aquaporin 1/cds = (59,868)/gb = L02914/gi = 193642/ug = Mm 18625/len = 2627/STRA = for | 233.7 | P | 373.4 | P | 468.3 | P | 391.1 |
| 3 | 93490_at | Cluster Incl AI841771 UI-M-AN0-acl-c-12-0-UI s1 Mus musculus cDNA, 3 end/clone = UI-M-AN0-acl-c-12-0-UI/ | 588 | P | 536 | P | 751.6 | P | 575.8 |
| | | clone_end = 3/gb = AI841771/gi = 5475984/ug = Mm 19055/len = 479/STRA = for | | | | | | | |
| 3 | 93500_at | Cluster Incl M63245 Mus musculus amino levulinate synthase (ALAS-H) mRNA, 3 end/cds = (0,1369)/gb = M63245/ | 568.8 | P | 726.4 | P | 788.3 | P | 590.6 |
| | | gi = 1220401/ug = Mm 19143/len = 1448/STRA = for | | | | | | | |
| 3 | 93511_at | Cluster Incl L38971 Integral membrane protein 2/cds = (147,938)/gb = L38971/gi = 624777/ug = Mm 193/len = 1635/ | 2760.5 | P | 3294.6 | P | 3016.2 | P | 3080.1 |
| | | STRA = for | | | | | | | |
| 3 | 93540_at | Cluster Incl L13290 Mus musculus ADP-ribosylarginine hydrolase mRNA, complete cds/cds = (0,1088)/gb = L13290/ | 477.3 | P | 658 | P | 658.1 | P | 629.6 |
| | | gi = 402489/ug = Mm 20047/len = 1089/STRA = for | | | | | | | |
| 3 | 93732_f_at | Cluster Incl AF104358.Mus musculus synectin mRNA, complete cds/cds = (72,1073)/gb = AF104358/gi = 4219017/ | 866.7 | P | 903.8 | P | 737.4 | P | 809.9 |
| | | ug = Mm 20945/len = 1600/STRA = for | | | | | | | |
| 3 | 93736_at | Cluster Incl AF090686: Mus musculus transcobalmin II (Tcn2) mRNA, complete cds/cds = (127,1419)/gb = AF090686/ | 277.5 | P | 372.5 | P | 568.5 | P | 349.7 |
| | | gi = 3659876/ug = Mm.20948/len = 1878/STRA = for | | | | | | | |
| 3 | 93767_i_at | Cluster Incl AW124052 UI-M-BH2.1-apn-e-06-0-UI s1 Mus musculus cDNA, 3 end/clone = UI-M-BH2.1-apn-e-06-0-UI/ | 1977.4 | P | 2124.9 | P | 2386.8 | P | 2279.7 |
| | | clone_end = 3/gb = AW124052/gi = 6099582/ug = Mm 21183/len = 246/STRA = for | | | | | | | |
| 3 | 93780_at | Cluster Incl AW060827 UI-M-BH1-amx-b-12-0-UI s1 Mus musculus cDNA, 3 end/clone = UI-M-BH1-amx-b-12-0-UI/ | 487 | P | 490 | P | 476.1 | P | 336.8 |
| | | clone_end = 3/gb = AW060827/gi = 6008578/ug = Mm 2125/len = 567/STRA = for | | | | | | | |
| 3 | 93966_at | Cluster Incl AI849706 UI-M-AL1-ahl-h-11-0-UI s1 Mus musculus cDNA, 3 end/clone = UI-M-AL1-ahl-h-11-0-UI/ | 559.1 | P | 749.5 | P | 877.5 | P | 694.1 |
| | | clone_end = 3/gb = AI849706/gi = 5493612/ug = Mm 21634/len = 217/STRA = for | | | | | | | |
| 3 | 94022_at | Cluster Incl AI844754 UI-M-AL1-ahq-e-04-0-UI s1 Mus musculus cDNA, 3 end/clone = UI-M-AL1-ahq-e-04-0-UI/ | 1045.7 | P | 1255 | P | 1175 | P | 1128.5 |
| | | clone_end = 3/gb = AI844754/gi = 5488660/ug = Mm 21859/len = 290/STRA = for | | | | | | | |
| 3 | 94244_at | Cluster Incl AW123807: UI-M-BH2.1-apm-f-02-0-UI s1 Mus musculus cDNA, 3 end/clone = UI-M-BH2.1-apm-f-02-0-UI/ | 591.1 | P | 618 | P | 846.8 | P | 689.1 |
| | | clone_end = 3/gb = AW123807/gi = 6099337/ug = Mm 22360/len = 226/STRA = for | | | | | | | |
| 3 | 94248_at | Cluster Incl M62419 Mouse clathrin-associated protein (AP47) mRNA, complete cds/cds = (72,1343)/gb = M62419/ | 454.7 | P | 853.5 | P | 833.9 | P | 741.1 |
| | | gi = 191985/ug = Mm 2237/len = 1966/STRA = for | | | | | | | |
| 3 | 94290_at | Cluster Incl AW124346: UI-M-BH2.1-apq-g-05-0-UI s1 Mus musculus cDNA, 3 end/clone = UI-M-BH2.1-apq-g-05-0-UI/ | 435.1 | P | 372.4 | P | 515.4 | P | 385.3 |
| | | clone_end = 3/gb = AW124346/gi = 6099876/ug = Mm 22577/len = 235/STRA = for | | | | | | | |
| 3 | 94833_at | Cluster Incl M91380: Follistatin-like/cds = (79,999)/gb = M91380/gi = 349005/ug = Mm 22763/len = 2823/STRA = for | 2013 | P | 2257.3 | P | 2195.5 | P | 2045.2 |
| 3 | 94842_at | Cluster Incl AI853630 UI-M-BH0-ajp-h-03-0-UI s1 Mus musculus cDNA, 3 end/clone = UI-M-BH0-ajp-h-03-0-UI/ | 1990.3 | P | 2175.1 | P | 2079.7 | P | 1853.1 |
| | | clone_end = 3/gb = AI853630/gi = 5497536/ug = Mm 22876/len = 547/STRA = for | | | | | | | |
| 3 | 94920_at | Cluster Incl AI854028: UI-M-BH0-ajm-o-01-UI.s1 Mus musculus cDNA, 3 end/clone = UI-M-BH0-ajm-c-01-0-UI/ | 325 | P | 395.1 | P | 392.5 | P | 368.7 |
| | | clone_end = 3/gb = AI854028 = 5497934/ug = Mm.24187/len = 439/STRA = for | | | | | | | |
| 3 | 95449_at | Cluster Incl AW049793: UI-M-BH1-anm-f-04-0-UI.s1 Mus musculus cDNA, 3 end/clone = UI-M-BH1-anm-f-04-0-UI/ | 411.3 | P | 464.2 | P | 338.1 | P | 419 |
| | | clone_end = 3/gb = AW049793/gi = 5910322/ug = Mm.24635/len = 569/STRA = for | | | | | | | |
| 3 | 95518_at | Cluster Incl AW122893: UI-M-BH2-apa-d-07-0-UI s1 Mus musculus cDNA, 3 end/clone = UI-M-BH2.1-apa-d-07-0-UI/ | 1028.7 | P | 1317 | P | 1508.3 | P | 792.1 |
| | | clone_end = 3/gb = AW122893/gi = 6098423/ug = Mm 25311/len = 217/STRA = for | | | | | | | |
| 3 | 96037_at | Cluster Incl X61454 M. musculus mRNA for I3 clone/cds = (0,224)/gb = X61454/gi = 51477/ug = Mm 2585/len = 449/STRA = for | 996.2 | P | 817.6 | P | 959.5 | P | 685.3 |
| 3 | 96082_at | Cluster Incl AI850644 UI-M-BG1-aik-f-01-0-UI s1 Mus musculus cDNA, 3 end/clone = UI-M-BG1-aik-f-01-0-UI/ | 874.8 | P | 966.6 | P | 889.5 | P | 949.2 |
| | | clone_end = 3/gb = AI850644/gi = 5494550/ug = Mm 26614/len = 374/STRA = for | | | | | | | |
| 3 | 96614_at | Cluster Incl AA783368 vw52e04 r1 Mus musculus cDNA, 5 end/clone = IMAGE-1247550/clone_end = 5/gb = AA763388/ | 157 | P | 171.3 | P | 195.7 | P | 220.9 |
| | | gi = 2813115/ug = Mm 27112/len = 450/STRA = for | | | | | | | |
| 3 | 96615_at | Cluster Incl AI840137 UI-M-AO0-acc-b-01-UI s1 Mus musculus cDNA, 3 end/clone = UI-M-AO0-acc-b-07-0-UI/ | 804.6 | P | 903.9 | P | 908.6 | P | 907.6 |
| | | clone_end = 3/gb = AI840137/gi = 5474350/ug = Mm 27114/len = 390/STRA = for | | | | | | | |
| 3 | 96662_at | Cluster Incl AI847054 UI-M-AP1-agn-a-07-0-UI s1 Mus musculus cDNA, 3 end/clone = UI-M-AP1-agn-a-07-0-UI/ | 273.5 | P | 317.8 | P | 305.9 | P | 287.8 |
| | | clone_end = 3/gb = AI847054/gi = 5490960/ug = Mm 27363/len = 389/STRA = for | | | | | | | |
| 3 | 97235_f_at | Cluster Incl AW124988 UI-M-BH2 1-apv-d-12-0-UI s1 Mus musculus cDNA, 3 end/clone = UI-M-BH2.1-apv-d-12-0-UI/ | 171.6 | P | 388.6 | P | 425.9 | P | 398 |
| | | clone_end = 3/gb = AW124988/gi = 6100518/ug = Mm 27822/len = 353/STRA = for | | | | | | | |
| 3 | 97800_at | Cluster Incl AI837308 UI-M-AK0-adf-a-09-0-UI s1 Mus musculus cDNA, 3 end/clone = UI-M-AK0-adf-a-09-0-UI/ | 1021.9 | P | 1417.7 | P | 1417.7 | P | 1330.1 |
| | | clone_end = 3/gb = AI837308/gi = 5471521/ug = Mm.28123/len = 287/STRA = for | | | | | | | |
| 3 | 98908_at | Cluster Incl AW125510 UI-M-BH2-aqm-e-12-0-UI s1 Mus musculus cDNA, 3 end/clone = UI-M-BH2-aqm-e-12-0-UI/ | 1165.6 | P | 1246.5 | P | 1525.7 | P | 1192.9 |
| | | clone_end = 3/gb = AW125510/gi = 6101040/ug = Mm 28586/len = 389/STRA = for | | | | | | | |
| 3 | 94501_at | Cluster Incl AI835784 UI-M-AI0-aaq-b-10-0-UI s1 Mus musculus cDNA, 3 end/clone = UI-M-AI0-aaq-b-10-0-UI/ | 1395.4 | P | 1518.8 | P | 1909.9 | P | 1252 |
| | | clone_end = 3/gb = AI835784/gi = 5469997/ug = Mm 28948/len = 395/STRA = for | | | | | | | |
| 3 | 95084_f_at | Cluster Incl AI844995 UI-M-BG0-ahu-e-03-0-UI s1 Mus musculus cDNA, 3 end/clone = UI-M-BG0-ahu-e-03-0-UI/ | 360.9 | P | 526.4 | P | 475.2 | P | 375.3 |
| | | clone_end = 3/gb = AI844995/gi = 5488901/ug = Mm 29256/len = 218/STRA = for | | | | | | | |
| 3 | 95117_at | Cluster Incl U04710 Insulin-like growth factor 2 receptor/cds = (132,7580)/gb = U04710/gi = 451552/ug = Mm 2938/len = 8877/ | 182.8 | P | 150.7 | P | 269.4 | P | 198.9 |
| | | STRA = for | | | | | | | |
| 3 | 95139_at | Cluster Incl AW122075 UI-M-BH2-aoo-f-04-0-UI s1 Mus musculus cDNA, 3 end/clone = UI-M-BH2-aoo-f-04-0-UI/ | 374.1 | P | 386.5 | P | 341.6 | P | 399.3 |
| | | clone_end = 3/gb = AW122075/gi = 6097482/ug = Mm 29432/len = 463/STRA = for | | | | | | | |
| 3 | 95146_at | Cluster Incl AI839280: UI-M-AO0-acb-b-06-UI s1 Mus musculus cDNA, 3 end/clone = UI-M-AO0-acb-b-06-0-UI/ | 541.4 | P | 421.7 | P | 656.5 | P | 544.7 |
| | | clone_end = 3/gb = AI839280/gi = 5473493/ug = Mm 29455/len = 265/STRA = for | | | | | | | |
| 3 | 95683_g_at | Cluster Incl AB026432 Mus musculus mRNA for XPE UV-damaged DNA binding factor, complete cds/cds = (81,3503)/ | 1450.2 | P | 1890.9 | P | 1736.4 | P | 1679.9 |
| | | gb = AB026432/gi = 5931595/ug = Mm.29623/len = 4171/STRA = for | | | | | | | |
| 3 | 96255_at | Cluster Incl AF067395 Mus musculus NIX (Nix) mRNA, nuclear gene encoding mitochondrial protein, complete cds/ | 803.8 | P | 1017.6 | P | 794.4 | P | 852.5 |
| | | cds = (99,755)/gb = AF067395/gi = 138823/ug = Mm 29820/len = 1285/STRA = for | | | | | | | |
| 3 | 96267_at | Cluster Incl AI846127 UI-M-AN1-afj-b-05-0-UI s2 Mus musculus cDNA, 3 end/clone = UI-M-AN1-afj-b-05-0-UI/ | 3260.5 | P | 3720.5 | P | 3227.5 | P | 3536.9 |
| | | clone_end = 3/gb = AI846127/gi = 5490033/ug = Mm 29842/len = 417/STRA = for | | | | | | | |
| 3 | 96344_at | Cluster Incl X61600 M musculus gene for beta-enolase/cds = (66,1370)/gb = X61600/gi = 50848/ug = Mm 29994/len = 1450/ | 2695.5 | P | 3866.6 | P | 3710.8 | P | 3607.2 |
| | | STRA = for | | | | | | | |
| 3 | 96351_at | Cluster Incl AI846097 UI-M-AK1-aew-c-06-0-UI s1 Mus musculus cDNA, 3 end/clone = UI-M-AK1-aew-c-06-0-UI/ | 293.9 | P | 285.3 | P | 264.6 | P | 259.4 |
| | | clone_end = 3/gb = AI846097/gi = 5489968/ug = Mm 30001/len = 183/STRA = for | | | | | | | |
| 3 | 96854_at | Cluster Incl AJ010391 Mus musculus copa gene, partial/cds = (0,94)/gb = AJ010391/gi = 5763780/ug = Mm 30041/len = 95/ | 867.5 | P | 956.8 | P | 1182.7 | P | 876.3 |
| | | STRA = for | | | | | | | |
| 3 | 96916_at | Cluster Incl AW046793 UI-M-BH1-alz-b-05-0-UI.s1 Mus musculus cDNA, 3 end/clone = UI-M-BH1-alz-b-05-0-UI/ | 706.7 | P | 739.4 | P | 680.7 | P | 628.2 |
| | | clone_end = 3/gb = AW046793/gi = 5907322/ug = Mm 30152/len = 237/STRA = for | | | | | | | |
| 3 | 96954_at | Cluster Incl AW122567 UI-M-BH2-2-aox-f-01-0-UI s1 Mus musculus cDNA, 3 end/clone = UI-M-BH2 2-aox-f-01-0-UI/ | 563.5 | P | 542.4 | P | 601.5 | P | 507.6 |
| | | clone_end = 3/gb = AW122567/gi = 6098092/ug = Mm 30212/len = 391/STRA = for | | | | | | | |
| 3 | 96956_at | Cluster Incl AW125526 UI-M-BH2.2-aqm-g-04-0-UI.s1 Mus musculus cDNA, 3 end/clone = UI-M-BH2.2-aqm-g-04-0-UI/ | 240.8 | P | 2380.1 | P | 2578 | P | 2339.6 |
| | | clone_end = 3/gb = AW125526/gi = 6101058/ug = Mm 30215/len = 443/STRA = for | | | | | | | |
| 3 | 97447_at | Cluster Incl AI836718 UI-M-AJ0-aax-d-11-0-UI.s1 Mus musculus cDNA, 3 end/clone = UI-M-AJ0-aax-d-11-0-UI/ | 1040.3 | P | 1041.6 | P | 1076.2 | P | 1031.1 |
| | | clone_end = 3/gb = AI836718/gi = 5470931/ug = Mm 30244/len = 276/STRA = for | | | | | | | |
| 3 | 97509_f_at | Cluster Incl U22324 Mus musculus fibroblast growth factor receptor-1 mRNA, long isoform precursor, complete cds/ | 361.3 | P | 508.1 | P | 583.2 | P | 443.1 |
| | | cds = (57,2525)/gb = U22324/gi = 722340/ug = Mm 3157/len = 2526/STRA = for | | | | | | | |
| 3 | 97526_at | Cluster Incl AW123294: UI-M-BH2 1-apg-h-02-0-UI s1 Mus musculus cDNA, 3 end/clone = UI-M-BH2 1-apg-h-02-0-UI/ | 279.3 | P | 253.5 | P | 398.2 | P | 219.6 |
| | | clone_end = 3/gb = AW123294/gi = 6098824/ug = Mm 3256/len = 380/STRA = for | | | | | | | |
| 3 | 98042_at | Cluster Incl AW049787 UI-M-BH1-anm-e-03-0-UI s1 Mus musculus cDNA, 3 end/clone = UI-M-BH1-anm-e-03-0-UI/ | 552.4 | P | 590.9 | P | 434.7 | P | 389.5 |
| | | clone_end = 3/gb = AW049787/gi = 5910316/ug = Mm 34126/len = 434/STRA = for | | | | | | | |
| 3 | 98097_r_at | Cluster Incl AI840219: UI-M-AI0-aal-g-03-0-UI s2 Mus musculus cDNA, 3 end/clone = UI-M-Al0-aal-g-03-0-UI/ | 125.1 | P | 132.8 | P | 124.6 | P | 144.4 |
| | | gb = AI840219/gi = 5474432/ug = Mm.34708/len = 371/STRA = for | | | | | | | |
| 3 | 98126_s_at | Cluster Incl X67140: Atpase, Ca++ transporting, cardiac muscle, fast twitch 1/cds = (0,548)/gb = X67140/gi = 509146/ | 1584.8 | P | 2866.5 | P | 2785.2 | P | 2923.7 |
| | | ug = Mm 35134/len = 855/STRA = for | | | | | | | |
| 3 | 98518_f_at | Cluster Incl AI604345 vv70f10.x1 Mus musculus cDNA, 3 end/clone = IMAGE-1227787/clone_end = 3/gb = AI604345/ | 4929.2 | P | 5380 | P | 5229.6 | P | 4782.1 |
| | | gi = 4613512/ug = Mm.35806/len = 397/STRA = for | | | | | | | |
| 3 | 98519_r_at | Cluster Incl AI604345 vv70f10 x1 Mus musculus cDNA, 3 end/clone = IMAGE-1227787/clone_end = 3/gb = AI604345/ | 782.9 | P | 1171.6 | P | 1096 | P | 736.5 |
| | | gi = 4613512/ug = Mm.35806/len = 397/STRA = for | | | | | | | |
| 3 | 98624_at | Cluster Incl X75316 M musculus seb4 mRNA/cds = (235,948)/gb = X75316/gi = 407467/ug = Mm.3865/len = 1908/STRA = for | 150.6 | P | 259.6 | P | 316 | P | 338.7 |
| 3 | 98630_at | Cluster Incl AW106745 um33b04 y1 Mus musculus cDNA, 5 end/clone = IMAGE-2236303/clone_end = 5/gb = AW106745/ | 409.7 | P | 320.7 | P | 337.9 | P | 353.7 |
| | | gi = 6077545/ug = Mm 38792/len = 600/STRA = for | | | | | | | |
| 3 | 99103_at | Cluster Incl AF036341 Mus musculus interferon regulatory factor-3 (IRF3) gene, partial cds/cds = (0,247)/gb = AF036341/ | 222 | P | 261.7 | P | 262.1 | P | 276.4 |
| | | gi = 2674204/ug = Mm 3960/len = 248/STRA = for | | | | | | | |
| 3 | 99142_at | Cluster Incl AI834895: UI-M-ANI-afe-d-08-0-UI.s1 Mus musculus cDNA, 3 end/clone = UI-M-AN1-afe-d-08-0-UI/ | 600.4 | P | 798 | P | 754.7 | P | 617.2 |
| | | clone_end = 3/gb = AI834895/gi = 5469108/ug = Mm 41405/len = 436/STRA = for | | | | | | | |
| 3 | 99153_at | Cluster Incl AI854607: UI-M-BH0-ake-c-10-0-UI.s1 Mus musculus cDNA, 3 end/clone = UI-M-BH0-ake-c-10-0-UI/ | 188.8 | P | 275.3 | P | 240.1 | P | 291 |
| | | clone_end = 3/gb = AI854607/gi = 5498513/ug = Mm 41574/len = 489/STRA = for | | | | | | | |
| 3 | 99161_at | Cluster Incl AI843359: UI-M-AQ1-adz-d-05-0-UI s1 Mus musculus cDNA, 3 end/clone = UI-M-AQ1-adz-d-05-0-UI/ | 1265.3 | P | 1371.8 | P | 1684.4 | P | 1616.2 |
| | | clone_end = 3/gb = AI843359/gi = 5477572/ug = Mm 41665/len = 425/STRA = for | | | | | | | |
| 3 | 99188_at | Cluster Incl AA615161 vn96a04 r1 Mus musculus cDNA, 5 end/clone = IMAGE-1039758/clone_end = 5/gb = AA615161/ | 552.9 | P | 880.4 | P | 806.9 | P | 606.9 |
| | | gi = 2502389/ug = Mm 41890/len = 495/STRA = for | | | | | | | |
| 3 | 99667_at | Cluster Incl U08439 Mus musculus Balb/c cytochrome c oxidase subunit VIaH mRNA, complete cds/cds = (57,350)/ | 561.6 | P | 876.8 | P | 879.3 | P | 914.1 |
| | | gb = U08439/gi = 475704/ug = Mm.43824/len = 466/STRA = for | | | | | | | |
| 3 | 99675_at | Cluster Incl AI839766 UI-M-AN0-acq-e-09-0-UI.s1 Mus musculus cDNA, 3 end/clone = UI-M-AN0-acq-e-09-0-UI/ | 437.2 | P | 416.1 | P | 360.8 | P | 525.9 |
| | | clone_end = 3/gb = AI839766/gi = 5474019/ug = Mm 44202/len = 366/STRA = for | | | | | | | |
| 3 | 100057_at | Cluster Incl AI838320 UI-M-AO0-aby-f-05-0-UI s1 Mus musculus cDNA, 3 end/clone = UI-M-AO0-aby-f-05-0-UI/ | 272.3 | P | 461.2 | P | 268.9 | P | 304.9 |
| | | clone_end = 3/gb = AI838320/gi = 5472533/ug = Mm 4467/len = 529/STRA = for | | | | | | | |
| 3 | 100084_at | Cluster Incl X60671 M musculus mRNA for ezrin/cds = (95,1855)/gb = X60671/gi = 50880/ug = Mm 4451/len = 2701/ | 730.2 | P | 859.4 | P | 1171 | P | 985.4 |
| | | STRA = for | | | | | | | |
| 3 | 100571_at | Cluster Incl AW123934 UI-M-BH2.3-aqc-b-11-0-UI s1 Mus musculus cDNA, 3 end/clone = UI-M-BH2 3-aqc-b-11-0-UI/ | 700.4 | P | 848.3 | P | 873.4 | P | 677.9 |
| | | clone_end = 3/gb = AW123934/gi = 6099464/ug = Mm 5856/len = 237/STRA = for | | | | | | | |
| 3 | 100574_f_at | Cluster Incl L09104 Glucose phosphate isomerase 1 complex/cds = (2066,2938)/gb = L09104/gi = 309267/ug = Mm 589/ | 3192.9 | P | 4066.8 | P | 4192.1 | P | 4128.1 |
| | | len = 3194/STRA = for | | | | | | | |
| 3 | 100600_at | Cluster Incl M58661 CD24a antigen/cds = (72,302)/gb = M58661/gi = 198985/ug = Mm 6417/len = 1800/STRA = for | 3639.6 | P | 4078.1 | P | 4078.7 | P | 3613.8 |
| 3 | 100630_f_at | Cluster Incl AA241764.mw26g09 r1 Mus musculus cDNA, 5 end/clone = IMAGE-671872/clone_end = 5/gb = AA241764/ | 1525.1 | P | 1622.6 | P | 1980.4 | P | 1505.9 |
| | | gi = 1872796/ug = Mm 667/len = 240/STRA = for | | | | | | | |
| 3 | 101029_f_at | Cluster Incl M15501 Actin, alpha, cardiac/cds = (0,1127)/gb = M15501/gi = 191653/ug = Mm 686/len = 1226/STRA = for | 3303.3 | P | 3866.6 | P | 4977.5 | P | 3540.6 |
| 3 | 101047_at | Cluster Incl AW123697 UI-M-BH2 1-apl-c-11-0-UI s1 Mus musculus cDNA, 3 end/clone = UI-M-BH2 1-apl-c-11-0-UI/ | 1624 | P | 2039.8 | P | 1645.1 | P | 1730 |
| | | clone_end = 3/gb = AW123697/gi = 6099227/ug = Mm.7/len = 413/STRA = for | | | | | | | |
| 3 | 101055_at | Cluster Incl J05261 Protective protein for beta-galactosidase/cds = (213,1637)/gb = J05261/gi = 200492/ug = Mm 7046/ | 842.4 | P | 1040.1 | P | 1273.5 | P | 964 |
| | | len = 1987/STRA = for | | | | | | | |
| 3 | 101102_at | Cluster Incl AJ223156 Mus musculus alpha4 gene, exon2 and join CDS/cds = (0,831)/gb = AJ223156/gi = 3127824/ | 433.8 | P | 457.6 | P | 494.2 | P | 420.2 |
| | | ug = Mm 7454/len = 1132/STRA = for | | | | | | | |
| 3 | 101109_at | Cluster Incl U43512 Dystroglycan 1/cds = (0,825)/gb = U43512/gi = 1155350/ug = Mm 7524/len = 3170/STRA = for | 693.6 | P | 1273.7 | P | 1081.3 | P | 646.8 |
| 3 | 101517_at | Cluster Incl X81058: M musculus tex261 mRNA/cds = (35,625)/gb = X81058/gi = 531527/ug = Mm 848/len = 1398/STRA = for | 608.2 | P | 779.5 | P | 721.7 | P | 790.9 |
| 3 | 101973_at | Cluster Incl Y15163 Mus musculus mRNA for mrg1 protein/cds = (219,1028)/gb = Y15163/gi = 3059126/ug = Mm.9524/ | 512.4 | P | 417.5 | P | 857.3 | P | 482.1 |
| | | len = 1943/STRA = for | | | | | | | |
| 3 | 101995_at | Cluster Incl U40930 Mus musculus oxidative stress-induced protein mRNA, complete cds/cds = (33,1361)/gb = U40930/ | 663.6 | P | 927.4 | P | 878.7 | P | 861.4 |
| | | gi = 1280523/ug = Mm 9846/len = 2000/STRA = for | | | | | | | |
| 3 | 102024_at | Cluster Incl AF000581 Mus musculus p300/CBP/Co-Integrator protein mRNA, complete cds/cds = (109,4305)/ | 76.6 | P | 91.1 | P | 65.6 | P | 98.8 |
| | | gb = AF000581/gi = 2934905/ug = Mm 1011/len = 4609/STRA = for | | | | | | | |
| 3 | 102043_at | Cluster Incl AF047704 Mus musculus tuftelin mRNA, complete cds/cds = (22,1194)/gb = AF047704/gi = 2920655/ | 289.5 | P | 541 | P | 507.4 | P | 475.3 |
| | | ug = Mm 10214/len = 2580/STRA = for | | | | | | | |
| 3 | 102063_at | Cluster Incl AF079535: Mus musculus 3-phosphoinositide dependent protein kinase-1 mRNA, complete cds/cds = (91,1770)/ | 231.9 | P | 187.1 | P | 212.7 | P | 213.2 |
| | | gb = AF079535/gi = 4028126/ug = Mm 10504/len = 1923/STRA = for | | | | | | | |
| 3 | 102336_at | Cluster Incl AF060565: Mus musculus RW1 protein mRNA, complete cds/cds = (28,5517)/gb = AF060565/gi = 3091277/ | 520 | P | 611.6 | P | 626.8 | P | 485.8 |
| | | ug = Mm 10808/len = 6271/STRA = for | | | | | | | |
| 3 | 102382_at | Cluster Incl AB014494: Aryl hydrocarbon receptor nuclear translocator-like/cds = (406,2286)/gb = AB014494/gi = 3402482/ | 113.1 | P | 92.7 | P | 92.2 | P | 103.8 |
| | | ug = Mm.12177/len = 2792/STRA = for | | | | | | | |
| 3 | 102402_at | Cluster Incl AJ001261 Mus musculus mRNA for NIPSNAP2 protein/cds = (48,893)/gb = AJ001261/gi = 2769257/ | 834.6 | P | 1126.9 | P | 1099.9 | P | 968.9 |
| | | ug = Mm.12468/len = 1604/STRA = for | | | | | | | |
| 3 | 102413_at | Cluster Incl AW124311 UI-M-BH2.1-apq-d-04-0-UI.s1 Mus musculus cDNA, 3 end/clone = UI-M-BH2.1-apq-d-04-0-UI/ | 737.2 | P | 960.9 | P | 970.3 | P | 746.6 |
| | | clone_end = 3/gb = AW124311/gi = 6099841/ug = Mm 12607/len = 222/STRA = for | | | | | | | |
| 3 | 102426_at | Cluster Incl U93291 Mus musculus skeletal muscle calsequestrin mRNA, complete cds/cds = (179,1378)/gb = U93291/ | 1286.7 | P | 1492.5 | P | 1713.1 | P | 1347.9 |
| | | gi = 1931626/ug = Mm 12829/len = 1395/STRA = for | | | | | | | |
| 3 | 102797_at | Cluster Incl X95281: Mus musculus mRNA for retinal short-chain dehydrogenase/reductase/cds = (416,1324)/gb = X95281/ | 291.3 | P | 310 | P | 318.4 | P | 252.2 |
| | | gi = 3776074/ug = Mm 14063/len = 1754/STRA = for | | | | | | | |
| 3 | 103094_at | Cluster Incl AI036894 uh20g09.r1 Mus musculus cDNA, 5 end/clone = IMAGE-1746016/clone_end = 5/gb = AI036894/ | 382 | P | 432.4 | P | 442.4 | P | 363.6 |
| | | gi = 3260585/ug = Mm.1749/len = 163/STRA = for | | | | | | | |
| 3 | 103322_at | Cluster Incl L35034 Ribose 5-phosphate isomerase A/cds = (207,917)/gb = L35034/gi = 836673/ug = Mm 17905/len = 1763/ | 633 | P | 599.8 | P | 860.4 | P | 595.7 |
| | | STRA = for | | | | | | | |
| 3 | 103412_at | Cluster Incl AI847230.UI-M-AI1-afq-0-12-0-UI s1 Mus musculus CDNA, 3 end/clone = UI-M-AI1-afq-c-12-0-UI/clone_end = 3/ | 171.2 | P | 230.5 | P | 145.8 | P | 133.6 |
| | | gb = AI847230/gi = 5491136/ug = Mm 18830/len = 459/STRA = for | | | | | | | |
| 3 | 103581_at | Cluster Incl Y14004.Mus musculus mRNA for acyl-CoA thioesterase/cds = (8,1267)/gb = Y14004/gi = 2765361/ug = Mm.1978/ | 595.4 | P | 416.6 | P | 606.2 | P | 519.6 |
| | | len = 1574/STRA = for | | | | | | | |
| 3 | 103623_at | Cluster Incl L39790 Mus musculus fibrillin 2 (fbn2) gene, complete cds/cds = (0,8723)/gb = L39790/gi = 762830/ | 381.8 | P | 494 | P | 455.3 | P | 443 |
| | | ug = Mm 20271/len = 8724/STRA = for | | | | | | | |
| 3 | 103644_at | Cluster Incl D13139.Dipeptidase 1 (renal)/cds = (257,1489)/gb = D13139/gi = 220397/ug = Mm 20388/len = 1655/STRA = for | 146.1 | P | 337.1 | P | 372.1 | P | 212.3 |
| 3 | 103684_f_at | Cluster Incl AA959648 vw57102 s1 Mus musculus cDNA, 3 end/clone = IMAGE-1247931/clone_end = 3/gb = AA959648/ | 247.4 | P | 224.4 | P | 247.1 | P | 217.4 |
| | | gi = 3125548/ug = Mm.20693/len = 426/STRA = for | | | | | | | |
| 3 | 103675_at | Cluster Incl Y17793 Mus musculus mRNA for Dutt1 protein/cds = (253,5091)/gb = Y17793/gi = 3688435/ug = Mm.20832/ | 69.2 | P | 61.5 | P | 76.8 | P | 25.1 |
| | | len = 6922/STRA = for | | | | | | | |
| 3 | 103807_at | Cluster Incl AB012265: Mus musculus mRNA for wizl, complete cds/cds = (772,5457)/gb = AB012265/gi = 3551181/ | 276 | P | 323 | P | 373.1 | P | 230.9 |
| | | ug = Mm.20878/len = 6425/STRA = rev | | | | | | | |
| 3 | 103809_r_at | Cluster Incl AF063229: Dynein, cyctoplasmic, intermediate chain 1/cds = (141,2027)/gb = AF063229/gi = 3493586/ | 770.6 | P | 785.3 | P | 747.3 | P | 813.7 |
| | | ug = Mm 20893/len = 2586/STRA = for | | | | | | | |
| 3 | 103826_at | Cluster Incl AF086830 Mus musculus leukemia/lymphoma related factor LRF (Lrf) mRNA, complete cds/cds = (58,1755)/ | 634.8 | P | 588.1 | P | 660.1 | P | 676.9 |
| | | gb = AF086830/gi = 3599510/ug = Mm 20920/len = 1793/STRA = for | | | | | | | |
| 3 | 103904_at | Cluster Incl X81584 M musculus mRNA for Insulin-like growth factor binding protein-6/cds = (192,908)/gb = X81584/ | 504 | P | 709 | P | 531.7 | P | 644.1 |
| | | gi = 550386/ug = Mm.21373/len = 1138/STRA = for | | | | | | | |
| 3 | 104044_at | Cluster Incl AI853663 UI-M-BH0-ajq-f-08-0-UI s1 Mus musculus cDNA, 3 end/clone = UI-M-BH0-ajq-f-08-0-UI/ | 691.1 | P | 626.4 | P | 634.8 | P | 588.8 |
| | | clone_end = 3/gb = AI853663/gi = 5497609/ug = Mm.21480/len = 432/STRA = for | | | | | | | |
| 3 | 104059_at | Cluster Incl AI851751 UI-M-BH0-aim-c-03-0-UI.s1 Mus musculus cDNA, 3 end/clone = UI-M-BH0-aim-c-03-0-UI/ | 371.4 | P | 339.2 | P | 371.3 | P | 318.9 |
| | | clone_end = 3/gb = AI851751/gi = 5495657/ug = Mm.21553/len = 377/STRA = for | | | | | | | |
| 3 | 104082_at | Cluster Incl AI848325 UI-M-AH1-ago-b-05-0-UI s1 Mus musculus cDNA, 3 end/clone = UI-M-AH1-ago-b-05-0-UI | 556.4 | P | 623 | P | 648.1 | P | 508.6 |
| | | clone_end = 3/gb = AI848325/gi = 5492231/ug = Mm 21759/len = 559/STRA = for | | | | | | | |
| 3 | 104140_s_at | Cluster Incl AI846989 UI-M-AP1-agi-b-11-0-UI s1 Mus musculus cDNA, 3 end/clone = UI-M-AP1-agi-b-11-0-UI/ | 1104.2 | P | 922.9 | P | 1095.5 | P | 1052.8 |
| | | clone_end = 3/gb = AI846989/gi = 5490895/ug = Mm.22121/len = 137/STRA = for | | | | | | | |
| 3 | 104141_at | Cluster Incl AI850401 UI-M-BG1-aig-f-01-0-UI s1 Mus musculus cDNA, 3 end/clone = UI-M-BG1-aig-f-01-0-UI/ | 653.1 | P | 789.8 | P | 793.6 | P | 641.3 |
| | | clone_end = 3/gb = AI850401/gi = 5494307/ug = Mm.22123/len = 511/STRA = for | | | | | | | |
| 3 | 104148_at | Cluster Incl AA939571: vz51d08 r1 Mus musculus cDNA, 5 end/clone = IMAGE.1329999/clone_end = 5/gb = AA939571/ | 575.9 | P | 791.8 | P | 743.7 | P | 714.5 |
| | | gi = 3100348/ug = Mm 22183/len = 420/STRA = for | | | | | | | |
| 3 | 104314_at | Cluster Incl AI851206 UI-M-BH0-aix-f-05-0-UI s1 Mus musculus cDNA, 3 end/clone = UI-M-BH0-ajx-f-05-0-UI/clone_end = 3/ | 620.1 | P | 689.9 | P | 707.4 | P | 619.9 |
| | | gb = AI851206/gi = 5495112/ug = Mm.22405/len = 405/STRA = for | | | | | | | |
| 3 | 104331_at | Cluster Incl AA832935: uc93c01 r1 Mus musculus cDNA, 5 end/clone = IMAGE-1433184/clone_end = 5/gb = AA832935/ | 982.1 | P | 993.6 | P | 1168.2 | P | 903.6 |
| | | gi = 2906863/ug = Mm.22478/len = 391/STRA = for | | | | | | | |
| 3 | 104360_at | Cluster Incl AI841844 UI-M-AO0-acb-g-01-0-UI s2 Mus musculus cDNA, 3 end/clone = UI-M-AO0-acb-g-01-0-UI/ | 1372.5 | P | 1201.5 | P | 1376 | P | 974.2 |
| | | clone_end = 3/gb = AI841844/gi = 5476057/ug = Mm.22588/len = 507/STRA = for | | | | | | | |
| 3 | 104363_at | Cluster Incl AA763213: vv88f01.r1 Mus musculus cDNA, 5 end/clone = IMAGE-1229497/clone_end = 5/gb = AA763213/ | 2947.2 | P | 3211 | P | 3513.8 | P | 3454.6 |
| | | gi = 2812960/ug = Mm.22608/len = 309/STRA = rev | | | | | | | |
| 3 | 104366_at | Cluster Incl AW047831 UI-M-BH1-ais-c-04-0-UI s1 Mus musculus cDNA, 3 end/clone = UI-M-BH1-als-c-04-0-UI/ | 328.9 | P | 474.1 | P | 627 | P | 318.5 |
| | | clone_end = 3/gb = AW047831/gi = 5908360/ug = Mm.22616/len = 200/STRA = for | | | | | | | |
| 3 | 104555_at | Cluster Incl AA733946: vv21f05 r1 Mus musculus cDNA, 5 end/clone = IMAGE-1223073/clone_end = 5/gb = AA733946/ | 1029 | P | 1428.5 | P | 1513.8 | P | 1322.4 |
| | | gi = 2755613/ug = Mm.23786/len = 313/STRA = for | | | | | | | |
| 3 | 104607_at | Cluster Incl AF093624 Mus musculus Nap-like 1 protein (Nspl 1) gene, complete cds, tRNA-Sec gene, complete sequence, | 263.9 | P | 479.7 | P | 446.1 | P | 423.9 |
| | | and FosB protein (Fosb) gene, complete cds/cds = (164,1579)/gb = AF093624/gi = 4235385/ug = Mm.24142/len = 1938/ | | | | | | | |
| | | STRA = for | | | | | | | |
| 3 | 104614_at | Cluster Incl AI852765 UI-M-BH0-aly-c-05-0-UI s1 Mus musculus cDNA, 3 end/clone = UI-M-BH0-aly-c-05-0-UI/ | 1251.9 | P | 1478.1 | P | 1574.7 | P | 1271.2 |
| | | clone_end = 3/gb = AI852765/gi = 5496671/ug = Mm.24193/len = 401/STRA = for | | | | | | | |
| 3 | 104709_at | Cluster Incl AI843665 UI-M-AK1-aer-a-11-0-UI s1 Mus musculus cDNA, 3 end/clone = UI-M-AK1-aer-a-11-0-UI/ | 481.7 | P | 600.8 | P | 554.3 | P | 536.8 |
| | | clone_end = 3/gb = AI843665/gi = 5477878/ug = Mm.24426/len = 314/STRA = for | | | | | | | |
| 3 | 104760_at | Cluster Incl AI838527 UI-M-AL0-abp-a-12-0-UI s1 Mus musculus cDNA, 3 end/clone = UI-M-AL0-abp-a-12-0-UI/ | 629 | P | 699.3 | P | 878.8 | P | 743.3 |
| | | clone_end = 3/gb = AI838527/gi = 5472705/ug = Mm.24831/len = 305/STRA = for | | | | | | | |
| 3 | 94438_at | Cluster Incl AI852672 UI-M-BH0-aji-b-09-0-UI s1 Mus musculus cDNA, 3 end/clone = UI-M-BH0-aji-b-09-0-UI/clone_end = 3/ | 664.8 | P | 726.7 | P | 952.6 | P | 757.8 |
| | | gb = AI852672/gi = 5496578/ug = Mm 26550/len = 381/STRA = for | | | | | | | |
| 3 | 94952_at | Cluster Incl AI152780 ud91a10 rt Mus musculus cDNA, 5 end/clone = IMAGE-1478202/clone_end = 5/gb = AI152780/ | 845.3 | P | 884.9 | P | 969.4 | P | 1028.8 |
| | | gi = 3681249/ug = Mm.27118/len = 250/STRA = for | | | | | | | |
| 3 | 94968_at | Cluster Incl AW048793: UI-M-BH1-amg-c-06-0-UI s1 Mus musculus cDNA, 3 end/clone = UI-M-BH1-amg-c-06-0-UI/ | 1011.4 | P | 822.2 | P | 1126.9 | P | 953.4 |
| | | clone_end = 3/gb = AW048793/gi = 5909322/ug = Mm 27220/len = 335/STRA = for | | | | | | | |
| 3 | 95531_at | Cluster Incl AI854771 UI-M-BH0-aka-c-10-0-UI s1 Mus musculus cDNA, 3 end/clone = UI-M-BH0-aka-c-10-0-UI/ | 230.4 | P | 313.6 | P | 290.6 | P | 216.6 |
| | | clone_end = 3/gb = AI854771/gi = 5498677/ug = Mm.27633/len = 519/STRA = for | | | | | | | |
| 3 | 95547_at | Cluster Incl D89821 Mus musculus mRNA for RhoM, complete cds/cds = (84,716)/gb = D89821/gi = 1752824/ug = Mm 27701/ | 303.1 | P | 321.6 | P | 320.8 | P | 341.8 |
| | | len = 1166/STRA = for | | | | | | | |
| 3 | 95587_at | Cluster Incl AI837204 UI-M-AK0-add-f-07-0-UI.s1 Mus musculus cDNA, 3 end/clone = UI-M-AK0-add-f-07-0-UI/ | 364.9 | P | 383.2 | P | 384.1 | P | 376.2 |
| | | clone_end = 3/gb = AI837204/gi = 5471417/ug = Mm.27862/len = 287/STRA = for | | | | | | | |
| 3 | 95607_at | Cluster Incl X82457 M. musculus es64 mRNA/cds = (176,1516)/gb = X82457/gi = 951299/ug = Mm.27983/len = 2054/ | 459.9 | P | 524.5 | P | 523.1 | P | 493 |
| | | STRA = for | | | | | | | |
| 3 | 95625_at | Cluster Incl AI840149 UI-M-AO0-acc-c-09-0-UI.s1 Mus musculus cDNA, 3 end/clone = UI-M-AO0-acc-c-09-UI/ | 348 | P | 494.9 | P | 461.7 | P | 424.1 |
| | | clone_end = 3/gb = AI840149/gi = 5474362/ug = Mm 28056/len = 339/STRA = for | | | | | | | |
| 3 | 96140_at | Cluster Incl AF039021: Mus musculus pencentriolar material 1 (Pcm1) gene, partial cds/cds = (0,196)/gb = AF039021/ | 185 | P | 186.4 | P | 157.5 | P | 151.4 |
| | | gi = 3170134/ug = Mm.28192/len = 197/STRA = for | | | | | | | |
| 3 | 96154_at | Cluster Incl AA600645: vm77b04 r1 Mus musculus cDNA, 5 end/clone = IMAGE-1004239/clone_end = 5/gb = AA600645/ | 511.9 | P | 632.9 | P | 316 | P | 307 |
| | | gi = 2434270/ug = Mm.28280/len = 378/STRA = for | | | | | | | |
| 3 | 96156_at | Cluster Incl AA874329 vx79h02.r1 Mus musculus cDNA, 5 end/clone = IMAGE-1281459/clone_end = 5/gb = AA874329/ | 226.8 | P | 225.4 | P | 258.1 | P | 254.1 |
| | | gi = 2979018/ug = Mm.28311/len = 206/STRA = for | | | | | | | |
| 3 | 96186_at | Cluster Incl AI839288 UI-M-AO0-acb-c-08-0-UI s1 Mus musculus cDNA, 3 end/clone = UI-M-AO0-acb-c-08-0-UI/ | 535.3 | P | 594.9 | P | 751.3 | P | 847.2 |
| | | clone_end = 3/gb = AI839286/gi = 5473499/ug = Mm.28465/len = 258/STRA = for | | | | | | | |
| 3 | 96200_at | Cluster Incl AI838344 UI-M-AO0-aby-h-11-0-UI s1 Mus musculus cDNA, 3 end/clone = UI-M-AO0-aby-h-11-0-UI/ | 462.9 | P | 512.4 | P | 537.2 | P | 490.3 |
| | | clone_end = 3/gb = AI838344/gi = 5472557/ug = Mm.28595/len = 393/STRA = for | | | | | | | |
| 3 | 96731_at | Cluster Incl AA615100: vo34f04 r1 Mus musculus cDNA, 5 end/clone = IMAGE-1051807/clone_end = 5/gb = AA615100/ | 807.5 | P | 753.2 | P | 834.5 | P | 640.9 |
| | | gi = 2502328/ug = Mm 28869/len = 145/STRA = for | | | | | | | |
| 3 | 96747_at | Cluster Incl AW121294 UI-M-BH2 3-aog-d-09-0-UI s1 Mus musculus cDNA, 3 end/clone = UI-M-BH2 3-aog-d-09-0-UI/ | 426.9 | P | 457.6 | P | 658.6 | P | 430.7 |
| | | clone_end = 3/gb = AW121294/gi = 6096627/ug = Mm.28954/len = 235/STRA = for | | | | | | | |
| 3 | 96761_at | Cluster Incl AF109906: Mus musculus MHC class III region RD gene, partial cds; Bf, C2, G9A, NG22, G9, HSP70, HSP70, | 635.4 | P | 609.4 | P | 657.5 | P | 655.3 |
| | | HSC70t, and smRNP genes, complete cds; G7A gene, partial cds; and unknown genes/cds = (0,2123)/gb = AF 109906/ | | | | | | | |
| | | gi = 3986763/ug = Mm.29004/len = 212/STRA = for | | | | | | | |
| 3 | 96771_at | Cluster Incl AI1006228 ua88d09 r1 Mus musculus cDNA, 5 end/clone = IMAGE-1364561/clone_end = 5/gb = AI006228/ | 848.4 | P | 937.9 | P | 1016.3 | P | 932.7 |
| | | gi = 3215837/ug = Mm 29023/len = 382/STRA = rev | | | | | | | |
| 3 | 96837_at | Cluster Incl AI837492 UI-M-ALO-abr-f-11-0-UI s1 Mus musculus cDNA, 3 end/clone = UI-M-AL0-abr-f-11-0-UI/clone_end = 3/ | 1149.7 | P | 1150.5 | P | 1306.5 | P | 1073.8 |
| | | gb = AI837492/gi = 5471705/ug = Mm.29442/len = 181/STRA = for | | | | | | | |
| 3 | 97357_at | Cluster Incl AI426400 mm50h04 x1 Mus musculus cDNA, 3 end/clone = IMAGE-524983/clone_end = 3/gb = AI426400/ | 128.7 | P | 169.8 | P | 167.1 | P | 116.9 |
| | | gi = 4272326/ug = Mm 29635/len = 157/STRA = for | | | | | | | |
| 3 | 97929_r_at | Cluster Incl AW124782: UI-M-BH2.1-aqa-h-02-0-UI s1 Mus musculus cDNA, 3 end/clone = UI-M-BH2 1-aqa-h-02-0-UI/ | 759.8 | P | 884.1 | P | 786.9 | P | 799.7 |
| | | clone_end = 3/gb = AW124782/gi = 6100312/ug = Mm 30237/len = 484/STRA = for | | | | | | | |
| 3 | 97935_at | Cluster Incl AI842970 UI-M-AO1-aem-g-06-0-UI s1 Mus musculus cDNA, 3 end/clone = UI-M-AO1-aem-g-06-0-UI/ | 554.4 | P | 525.7 | P | 558.8 | P | 522.2 |
| | | clone_end = 3/gb = AI842970/gi = 5477183/ug = Mm 30252/len = 374/STRA = for | | | | | | | |
| 3 | 97994_at | Cluster Incl AI019193 ub20e12 r1 Mus musculus cDNA, 5 end/clone = IMAGE-1378318/clone_end = 5/gb = AI019193/ | 330.2 | P | 468 | P | 506.3 | P | 300.1 |
| | | gi = 3233529/ug = Mm 31630/len = 476/STRA = for | | | | | | | |
| 3 | 98027_at | Cluster Incl Z22923 Procollagen, type IX, alpha 2/cds = (383,2449)/gb = Z22923/gi = 311949/ug = Mm 3277/len = 2956/ | 367.7 | P | 360.5 | P | 496.1 | P | 365.1 |
| | | STRA = for | | | | | | | |
| 3 | 98426_at | Cluster Incl AF030001 Mus musculus major histocompatibility locus class III region-butyrophilin-like protein gene, partial | 189.4 | P | 102.3 | P | 178.9 | P | 136.7 |
| | | cds, Notch4, PBX2, RAGE, lysophatidic acid acyl transferase-alpha, palmitoyl-protein thioesterase 2 (PPT2), CREB-RP, | | | | | | | |
| | | and tenascin X/STRA = for | | | | | | | |
| 3 | 98435_at | Cluster Incl M74495 Mouse adenylosuccinate synthetase mRNA, complete cds/cds = (30,1403)/gb = M74495/gi = 404750/ | 222.1 | P | 227.4 | P | 263.3 | P | 242.1 |
| | | ug = Mm 3440/len = 1736/STRA = for | | | | | | | |
| 3 | 98454_at | Cluster Incl Y14771 Mus musculus mRNA for paralernmin/cds = (127,1278)/gb = Y14771/gi = 4456676/ug = Mm 34650/ | 544 | P | 761.4 | P | 521.7 | P | 459.8 |
| | | len = 1936/STRA = for | | | | | | | |
| 3 | 98460_at | Cluster Incl AJ237917 Mus musculus mRNA for fatso protein/cds = (37,1545)/gb = AJ237917/gi = 6102999/ug = Mm.34778/ | 553.1 | P | 664.6 | P | 652.7 | P | 585.3 |
| | | len = 3554/STRA = for | | | | | | | |
| 3 | 98476_at | Cluster Incl L40631 Ankyrin 3, epithelial/cds = (647,3931)/gb = L40631/gi = 710545/ug = Mm 3526/len = 5074/STRA = for | 202.8 | P | 228.4 | P | 288.8 | P | 177.8 |
| 3 | 98989_at | Cluster Incl AF057368 7-dehydrocholesterol reductase/cds = (39,1454)/gb = AF057368/gi = 3283173/ug = Mm 3747/ | 236.6 | P | 272.8 | P | 218.8 | P | 244.1 |
| | | len = 2377/STRA = rev | | | | | | | |
| 3 | 99009_at | Cluster Incl Z49204 Nicotinamide nucleotide transhydrogenase/cds = (68,3328)/gb = Z49204/gi = 840318/ug = Mm 3842/ | 268.8 | P | 430.3 | P | 457.5 | P | 271.3 |
| | | len = 3844/STRA = for | | | | | | | |
| 3 | 99024_at | Cluster Incl U32395 Mus musculus Max-interacting transcriptional repressor (Mad4) mRNA, complete cds/cds = (0,629)/ | 808 | P | 1052.8 | P | 1061 | P | 811.5 |
| | | gb = U32395/gi = 1184158/ug = Mm.38795/len = 630/STRA = for | | | | | | | |
| 3 | 99073_at | Cluster Incl Z47766 Cyclin F/cds = (39,2369)/gb = Z47766/gi = 833246/ug = Mm 4008/len = 2815/STRA = for | 362.1 | P | 284.2 | P | 289.7 | P | 306.8 |
| 3 | 99448_at | Cluster Incl X56123 Mouse mRNA for talin/cds = UNKNOWN/gb = X56123/gi = 54257/ug = Mm 4053/len = 8233/STRA = for | 379.4 | P | 257.4 | P | 485 | P | 442.9 |
| 3 | 99471_at | Cluster Incl AF158183 AF158183 Mus musculus cDNA/gb = AF158183/gi = 5499038/ug = Mm 41271/len = 1034/STRA = for | 707.1 | P | 738.4 | P | 861.1 | P | 781.9 |
| 3 | 100003_at | Cluster Incl D38216 Mouse RyR1 mRNA for skeletal muscle ryanodine receptor, partial cds/cds = (0,371)/gb = D38216/ | 219.9 | P | 252.9 | P | 238.5 | P | 245 |
| | | gi = 1030707/ug = Mm 4519/len = 486/STRA = for | | | | | | | |
| 3 | 100436_at | Cluster Incl M27008 Orosomucoid 1/cds = (32,655)/gb = M27008/gi = 531206/ug = Mm 4777/len = 768/STRA = for | 273.5 | P | 383.9 | P | 413.9 | P | 467.4 |
| 3 | 100440_f_at | Cluster Incl U76758 Ankyrin 1, erythroid/cds = (0,164)/gb = U76758/gi = 1698953/ug = Mm 4789/len = 191/STRA = for | 622.8 | P | 808.6 | P | 725.7 | P | 638.6 |
| 3 | 100457_at | Cluster Incl X84037: M musculus mRNA for E-selectin ligand-1/cds = (6,3533)/gb = X84037/gi = 673435/ug = Mm 488/ | 806 | P | 1000.6 | P | 861.4 | P | 964.5 |
| | | len = 3887/STRA = for | | | | | | | |
| 3 | 100486_at | Cluster Incl U60453 Mus musculus enhancer of zeste homolog 1 (Ezh1) mRNA, complete cds/cds = (108,2351)/ | 419 | P | 387.3 | P | 334.7 | P | 370 |
| | | gb = U60453/gi = 1575340/ug = Mm.5027/len = 3970/STRA = for | | | | | | | |
| 3 | 100509_at | Cluster Incl AW121012 UI-M-BH2.3-aod-h-06-0-UI.s1 Mus musculus cDNA, 3 end/clone = UI-M-BH2.3-aod-h-06-0-UI/ | 325.5 | P | 269.6 | P | 334.5 | P | 265.6 |
| | | clone_end = 3/gb = AW121012/gi = 6096345/ug = Mm 5181/len = 204/STRA = for | | | | | | | |
| 3 | 100893_at | Cluster Incl U27455 Serine palmitoyltransferase, long chain base subunit 2$$/cds = (27,1709)/gb = U27455/gi = 2267216/ | 521.1 | P | 408.5 | P | 499.5 | P | 591.4 |
| | | ug = Mm 565/len = 1893/STRA = rev | | | | | | | |
| 3 | 100896_at | Cluster Incl AJ006306 Mus musculus mRNA for skeletal muscle calcium channel, gamma-subunit/cds = (131,802) | 271.8 | P | 340.4 | P | 355.2 | P | 327.9 |
| | | gb = AJ006306/gi = 3157401/ug = Mm 57093/len = 803/STRA = for | | | | | | | |
| 3 | 101383_at | Cluster Incl AJ131711.Mus musculus mRNA for troponin T/cds = (1,789)/gb = AJ131711/gi = 4468629/ug = Mm 711/len = 907/ | 243.5 | P | 481.1 | P | 488.5 | P | 379 |
| | | STRA = for | | | | | | | |
| 3 | 101394_at | Cluster Incl AB024922: Mus musculus mRNA for gamma-sarcogiycan, complete cds/cds = (256,1131)/gb = AB024922/ | 191.3 | P | 228.7 | P | 230.7 | P | 211.6 |
| | | gi = 5771417/ug = Mm 72173/len = 1289/STRA = for | | | | | | | |
| 3 | 101451_at | Cluster Incl AF038939 Mus musculus zinc finger protein (Peg3) mRNA, complete cds/cds = (13,4726)/gb = AF038939/ | 1299.9 | P | 1048.4 | P | 692.9 | P | 1188.6 |
| | | gi = 2791677/ug = Mm 7952/len = 5560/STRA = for | | | | | | | |
| 3 | 101458_at | Cluster Incl D30743.Mouse mRNA for wee 1 kinase/cds = (292,2232)/gb = D30743/gi = 765088/ug = Mm.8108/len = 2270/ | 16.2 | P | −17.3 | P | 155.6 | P | −45.9 |
| | | STRA = for | | | | | | | |
| 3 | 101889_s_at | Cluster Incl U53228 RAR-related orphan receptor alpha/cds = (5,1576)/gb = U53228/gi = 1289325/ug = Mm 8858/len = 1657/ | 739.2 | P | 669.6 | P | 854.7 | P | 513.3 |
| | | STRA = for | | | | | | | |
| 3 | 101892_f_at | Cluster Incl Z67963: Fused toes/cds = (213,1091)/gb = Z67963/gi = 1067144/ug = Mm 88777/len = 2173/STRA = for | 289.3 | P | 266 | P | 273.2 | P | 279.2 |
| 3 | 101897_g_at | Cluster Incl M63697 CD1d2 antigen/cds = (293,1303)/gb = M63697/gi = 192473/ug = Mm 88779/len = 1728/STRA = for | 313.9 | P | 435.3 | P | 401.8 | P | 356.5 |
| 3 | 101943_at | Cluster Incl AA960259 ub61a12 s1 Mus musculus cDNA, 3 end/clone = IMAGE-1382206/clone_end = 3/gb = AA960259/ | 452 | P | 482.6 | P | 492.4 | P | 492.8 |
| | | gi = 3126159/ug = Mm 95636/len = 398/STRA = for | | | | | | | |
| 3 | 102223_at | Cluster Incl AF013715 Periplakin/cds = (0,331)/gb = AF013715/gi = 2992536/ug = Mm 10225/len = 776/STRA = for | 1951.8 | P | 1936.2 | P | 2046.5 | P | 1553.6 |
| 3 | 102224_at | Cluster Incl AF056187 Mus musculus insulin-like growth factor I receptor mRNA, complete cds/cds = (0,4121)/ | 656.8 | P | 578.3 | P | 1001.4 | P | 941.5 |
| | | gb = AF056187/gi = 3025893/ug = Mm 10226/len = 4500/STRA = rev | | | | | | | |
| 3 | 102304_f_at | Cluster Incl L28167 Zinc finger protein 61/cds = (927,2594)/gb = L28167/gi = 758660/ug = Mm,12832/len = 2606/STRA = for | 202.8 | P | 187.8 | P | 196.6 | P | 139.3 |
| 3 | 102866_at | Cluster Incl X94441: TEA domain family member 4/cds = (0,1283)/gb = X9441/gi = 1403383/ug = Mm.14744/len = 1284/ | 975.4 | P | 866 | P | 1200.5 | P | 963 |
| | | STRA = for | | | | | | | |
| 3 | 102867_at | Cluster Incl U51743: TEA domain family member 4/cds = (0,1138)/gb = U51743/gi = 1381555/ug = Mm.14774/len = 1339/ | 181.8 | P | 194.3 | P | 212.9 | P | 160.9 |
| | | STRA = for | | | | | | | |
| 3 | 102890_at | Cluster Incl U00677: Syntrophin, acidic 1/cds = (110,1621)/gb = U00677/gi = 404930/ug = Mm.1541/len = 2109/STRA = for | 819.8 | P | 844 | P | 950.9 | P | 874.8 |
| 3 | 102915_at | Cluster Incl Z48043 Coagulation factor II (thrombin) receptor-like 1/cds = (72,1271)/gb = Z48043/gi = 663020/ug = Mm.1614/ | 296.1 | P | 334.5 | P | 304.8 | P | 284.6 |
| | | len = 2713/STRA = for | | | | | | | |
| 3 | 103218_at | Cluster Incl J04761 Mouse housekeeping DXS253E (P3) and DXS254E (GdX) genes, 3 end, and complete cds/ | 419.9 | P | 376.1 | P | 320.8 | P | 457.5 |
| | | cds = (0,549)/gb = J04761/gi = 202256/ug = Mm.19931/len = 550/STRA = for | | | | | | | |
| 3 | 103466_at | Cluster Incl AA790056 vt78h04 r1 Mus musculus cDNA, 5 end/clone = IMAGE-1177303/clone_end = 5/gb = AA790058/ | 267.1 | P | 126.3 | P | 227.4 | P | 180 |
| | | gi = 2850176/ug = Mm.21783/len = 490/STRA = for | | | | | | | |
| 3 | 103534_at | Cluster Incl V00722 Mouse gene for beta-1-giobin/cds = (53,496)/gb = V00722/gi = 50155/ug = Mm.2329/len = 626/STRA = for | 3653.3 | P | 4909.1 | P | 4992.2 | P | 3808.4 |
| 3 | 103720_at | Cluster Incl AI449034 ms60f04.x1 Mus musculus cDNA, 3 end/clone = IMAGE-615967/clone_end = 3/gb = AI449034/ | 413.9 | P | 503.8 | P | 430.9 | P | 397 |
| | | gi = 4287173/ug = Mm.24194/len = 563/STRA = rev | | | | | | | |
| 3 | 103721_at | Cluster Incl AA592182: vo15b01 r1 Mus musculus cDNA, 5 end/clone = IMAGE-1049929/clone_end = 5/gb = AA592182/ | 158.1 | P | 271.4 | P | 301.5 | P | 99.6 |
| | | gi = 2405845/ug = Mm.24198/len = 494/STRA = for | | | | | | | |
| 3 | 103770_at | Cluster Incl AA794189 vu67d02 r1 Mus musculus cDNA, 5 end/clone = IMAGE-1196451/clone_end = 5/gb = AA794189/ | 263.3 | P | 301.6 | P | 392.4 | P | 442.7 |
| | | gi = 2857144/ug = Mm.24776/len = 500/STRA = for | | | | | | | |
| 3 | 103958_g_at | Cluster Incl X57349 M. musculus mRNA for transferrin receptor/cds = (0,2291)/gb = X57349/gi = 54914/ug = Mm 25560/ | 1929.2 | P | 2176.8 | P | 2197.3 | P | 2014.3 |
| | | len = 2292/STRA = for | | | | | | | |
| 3 | 103964_at | Cluster Incl U85259 Estrogen receptor related 1/cds = (166,1428)/gb = U85259/gi = 1916860/ug = Mm 2563/len = 2203/ | 521 | P | 620.2 | P | 574 | P | 611.3 |
| | | STRA = for | | | | | | | |
| 3 | 104188_at | Cluster Incl AI853703 UI-M-BH0-ajq-c-08-0-UI s1 Mus musculus cDNA, 3 end/clone = UI-M-BH0-ajq-c-08-0-UI/ | 287.3 | P | 298.8 | P | 346.6 | P | 300 |
| | | clone_end = 3/gb = AI853703/gi = 5497574/ug = Mm 27449/len = 485/STRA = for | | | | | | | |
| 3 | 104196_at | Cluster Incl AA711908 vu59e03 r1 Mus musculus cDNA, 5 end/clone = IMAGE-1195708/clone_end = 5/gb = AA711908/ | 211.4 | P | 230 | P | 192.9 | P | 249.3 |
| | | gi = 2721826/ug = Mm 27512/len = 474/STRA = for | | | | | | | |
| 3 | 104199_at | Cluster Incl AI646237 vv88b02.x1 Mus musculus cDNA, 3 end/clone = IMAGE-1229451/clone_end = 3/gb = AI646237/ | 476.6 | P | 678.4 | P | 569.6 | P | 473.8 |
| | | gi = 4724712/ug = Mm.27521/len = 347/STRA = for | | | | | | | |
| 3 | 104218_s_at | Cluster Incl AI507524 vl53f03 x1 Mus musculus cDNA, 3 end/clone = IMAGE-975965/clone_end = 3/gb = AI507524/ | 208.2 | P | 252.2 | P | 223.3 | P | 86.3 |
| | | gi = 4405375/ug = Mm.27913/len = 433/STRA = for | | | | | | | |
| 3 | 104250_at | Cluster Incl AW121353: UI-M-BH2 2-aom-c-12-0-UI s1 Mus musculus cDNA, 3 end/clone = UI-M-BH2.2-aom-c-12-0-UI/ | 416.2 | P | 432.3 | P | 402.8 | P | 456.8 |
| | | clone_end = 3/gb = AW121353/gi = 6096686/ug = Mm.28355/len = 562/STRA = for | | | | | | | |
| 3 | 104264_at | Cluster Incl AW122230.UI-M-BH2 2-aov-e-05-0-UI.s1 Mus musculus cDNA, 3 end/clone = UI-M-BH2.2aov-e-05-0-UI/ | 442 | P | 361 | P | 373.4 | P | 370.9 |
| | | clone_end = 3/gb = AW122230/gi = 6097693/ug = Mm.28458/len = 266/STRA = for | | | | | | | |
| 3 | 104436_at | Cluster Incl AJ250429 Mus musculus mRNA for IL-1L1 protein/cds = (112,579)/gb = AJ250429/gi = 6165412/ug = Mm.29261/ | 789.4 | P | 1026.6 | P | 875.8 | P | 828.7 |
| | | len = 1284/STRA = rev | | | | | | | |
| 3 | 104453_at | Cluster Incl AW046336.UI-M-BH1-ale-f-04-0-UI s1 Mus musculus cDNA, 3 end/clone = UI-M-BH1-ale-f-04-0-UI/ | 558.3 | P | 637.7 | P | 661.1 | P | 537.9 |
| | | clone_end = 3/gb = AW046336/gi = 5906865/ug = Mm.2947/len = 298/STRA = rev | | | | | | | |
| 3 | 104471_at | Cluster Incl AF006603 Mus musculus histone deacetylase mHDA2 mRNA, complete cds/cds = (0,3287)/gb = AF006603/ | 477.7 | P | 446.9 | P | 464.2 | P | 468.3 |
| | | gi = 4165860/ug = Mm.29854/len = 3651/STRA = for | | | | | | | |
| 3 | 104502_f_at | Cluster Incl AI414025 ma02g05.x1 Mus musculus cDNA, 3 end/clone = IMAGE-303416/clone_end = 3/gb = AI414025/ | 155.2 | P | 142.8 | P | 205.5 | P | 156.8 |
| | | gi = 4257529/ug = Mm.30608/len = 346/STRA = for | | | | | | | |
| 3 | 104638_at | Cluster Incl U31510 ADP-ribosyltransferase 1/cds = (279,1256)/gb = U31510/gi = 1209033/ug = Mm.31099/len = 1640/ | 116.3 | P | 247.7 | P | 113.9 | P | 293.8 |
| | | STRA = for | | | | | | | |
| 3 | 104673_at | Cluster Incl X65138 Eph receptor A4/cds = (57,3017)/gb = X65138/gi = 54083/ug = Mm.3249/len = 4242/STRA = for | 289.1 | P | 225.1 | P | 242 | P | 182.5 |
| 3 | 104697_at | Cluster Incl AW121127.UI-M-BH2.3-aoh-d-04-0-UI s1 Mus musculus cDNA, 3 end/clone = UI-M-BH2.3-aoh-d-04-0-UI/ | 132.5 | P | 157.3 | P | 289.7 | P | 257.9 |
| | | clone_end = 3/gb = AW121127/gi = 6096460/ug = Mm.33722/len = 402 = STRA = for | | | | | | | |
| 3 | 92259_at | Cluster Incl AI467526: ve34d12.x1 Mus musculus cDNA, 3 end/clone = IMAGE-820055/clone_end = 3/gb = AI467526/ | 1034.9 | P | 1022.6 | P | 963.1 | P | 1029.9 |
| | | gi = 4320863/ug = Mm.35645/len = 483/STRA = rev | | | | | | | |
| 3 | 92265_f_at | Cluster Incl AF042139 Mus musculus 60 kDa ribonucleoprotein Ro gene, partial cds/cds = (0,1375)/gb = AF042139/ | 746.3 | P | 789.9 | P | 536.3 | P | 731.9 |
| | | gi = 3098483/ug = Mm 35793/len = 1376/STRA = for | | | | | | | |
| 3 | 92371_at | Cluster Incl AI505355: vq02d11.x1 Mus musculus cDNA, 3 end/clone = IMAGE-1093077/clone_end = 3/gb = AI505355/ | 417.6 | P | 481.4 | P | 379.8 | P | 381.6 |
| | | gi = 4403206/ug = Mm.39968/len = 421/STRA = for | | | | | | | |
| 3 | 92407_at | Cluster Incl AJ012072.Mus musculus mRNA for myomesin/skelemin/cds = (158,5158)/gb = AJ012072/gi = 5419921/ | 119.3 | P | 310 | P | 314.3 | P | 352.7 |
| | | ug = Mm.4103/len = 5645/STRA = for | | | | | | | |
| 3 | 92466_at | Cluster Incl U85714 Mus musculus phospholipase C-beta-1b mRNA, complete cds/cds = (3,3524)/gb = U85714/ | 1228.6 | P | 911.8 | P | 988.3 | P | 984.3 |
| | | gi = 4099296/ug = Mm.42083/len = 3853/STRA = for | | | | | | | |
| 3 | 92933_at | Cluster Incl Z18537 POU domain, class 2, transcription factor 3/cds = (75,1286)/gb = Z18537/gi = 53514/ug = Mm.4904/ | 735.3 | P | 804.4 | P | 803.5 | P | 729.2 |
| | | len = 1267/STRA = for | | | | | | | |
| 3 | 92958_at | Cluster Incl AI849135: UI-M-AJ1-agy-b-09-0-UI.s1 Mus musculus cDNA, 3 end/clone = UI-M-AJ1-agy-b-09-0-UI/ | 561.9 | P | 575.5 | P | 830.6 | P | 371.6 |
| | | clone_end = 3/gb = AI849135/gi = 5493041/ug = Mm.4948/len = 384 = STRA = for | | | | | | | |
| 3 | 93193_at | Cluster Incl X15643.Adrenergic receptor, beta 2/cds = (0,1256)/gb = X15643/gi = 50100/ug = Mm.5598/len = 1257/STRA = for | 308.5 | P | 316.5 | P | 352.1 | P | 239 |
| 3 | 93235_at | Cluster Incl AI020029 ub24c02 r1 Mus musculus cDNA, 5 end/clone = IMAGE-1378658/clone_end = 5/gb = AI020029/ | 248.8 | P | 417 | P | 513 | P | 348.4 |
| | | gi = 3234365/ug = Mm.5833/len = 468/STRA = for | | | | | | | |
| 3 | 93383_at | Cluster Incl U32107 Procollagen, type VII, alpha 1/cds = (0,8834)/gb = U32107/gi = 2326167/ug = Mm.6200/len = 9235/ | 709.5 | P | 902.9 | P | 1167.1 | P | 769.4 |
| | | STRA = for | | | | | | | |
| 3 | 93451_at | Cluster Incl AW047919 UI-M-BH1-aln-c-05-0-UI.s1 Mus musculus cDNA, 3 end/clone = UI = M-BH1-aln-c-05-0-UI/ | 1225 | P | 1117 | P | 1031.4 | P | 741.3 |
| | | clone_end = 3/gb = AW047919/gi = 5908448/ug = Mm.6772/len = 413/STRA = for | | | | | | | |
| 3 | 93632_g_at | Cluster Incl X95761: M. musculus mRNA for new-Rhobin/cds = UNKNOWN/gb = X95761/gi = 1771307/ug = Mm.7634/ | 251.7 | P | 315 | P | 305.7 | P | 324.7 |
| | | len = 2031/STRA = rev | | | | | | | |
| 3 | 93681_at | Cluster Incl AW123618: UI-M-BH2.1-api-d-09-0-UI.s1 Mus musculus cDNA, 3 end/clone = UI-M-BH2.1-api-d-09-0-UI/ | 569.4 | P | 566.5 | P | 510.2 | P | 548.5 |
| | | clone_end = 3/gb = AW123618/gi = 6099148/ug = Mm 82649/len = 416/STRA = for | | | | | | | |
| 3 | 93903_at | Cluster Incl M84120 Mouse activin receptor (ActR IIB) mRNA, complete cds/cds = (43,1653)/gb = M84120/gi = 191668/ | 306.4 | P | 351.2 | P | 542.3 | P | 379 |
| | | ug = Mm.8940/len = 1708/STRA = for | | | | | | | |
| 3 | 93907_f_at | Cluster Incl M17551 Mus musculus clone MIA14 full-length intracisternal A-particle gag protein gene, complete cds, and pol | 3798.1 | P | 3845.9 | P | 4222.6 | P | 3845.3 |
| | | pseudogene, complete sequence/cds = (0,2483)/gb = M17551/gi = 193325/ug = Mm 89480/len = 2484/STRA = for | | | | | | | |
| 3 | 93908_f_at | Cluster Incl X16670 Mouse RNA for type IIB intracisternal A-particle (IAP) element encoding integrase, clone 106/ | 820.5 | P | 599.5 | P | 1090.1 | P | 747.5 |
| | | cds = (498,1883)/gb = X16670/gi = 51488/ug = Mm 89480/len = 1931/STRA = for | | | | | | | |
| 3 | 93909_f_at | Cluster Incl X04120 Intracisternal A particles,Thbd linked/cds = (0,2444)/gb = X04120/gi = 51502/ug = Mm 89480/len = 2445/ | 1370.3 | P | 1019.1 | P | 2022.3 | P | 1229.7 |
| | | STRA = for | | | | | | | |
| 3 | 94106_at | Cluster Incl AI853048 UI-M-BH0-ajn-f-11-0-UI s1 Mus musculus cDNA, 3 end/clone = UI-M-BH0-ajn-f-11-0-UI/ | 434.5 | P | 614.9 | P | 563 | P | 503.5 |
| | | clone_end = 3/gb = AI853048/gi = 5496954/ug = Mm 1024/len = 348/STRA = for | | | | | | | |
| 3 | 94802_at | Cluster Incl AI846678 UI-M-AN1-afi-d-11-0-UI.s1 Mus musculus cDNA, 3 end/clone = UI-M-AN1-afi-d-11-0-UI/ | 223.3 | P | 231.3 | P | 275.1 | P | 171.3 |
| | | clone_end = 3/gb = AI846678/gi = 5490584/ug = Mm 1835/len = 142/STRA = for | | | | | | | |
| 3 | 95322_g_at | Cluster Incl Y09588 Mus musculus mRNA for serotonin 4 receptor (m5-HT4(e)), splice vanant/cds = (2,1117)/gb = Y09588/ | 430.6 | P | 836.7 | P | 872.6 | P | 725.3 |
| | | gi = 3647302/ug = Mm 20440/len = 1266/STRA = for | | | | | | | |
| 3 | 95366_at | Cluster Incl L35599 Mus musculus Y-box binding protein mRNA, 3 end/cds = (0,825)/gb = L35599/gi = 532212/ | 220 | P | 257.4 | P | 200.8 | P | 209.5 |
| | | ug = Mm.22482/len = 2437/STRA = for | | | | | | | |
| 3 | 96578_r_at | Cluster Incl AI853019 UI-M-BHo-ajn-d-04-0-UI s1 Mus musculus cDNA, 3 end/clone = UI-M-BH0-ajn-d-04-0-UI/ | 358.1 | P | 477.6 | P | 443.7 | P | 345.3 |
| | | clone_end = 3/gb = AI853019/gi = 5496925/ug = Mm 3021/len = 250/STRA for | | | | | | | |
| 3 | 97116_at | Cluster Incl AW123210 UI-M-BH2.1-apg-d-05-0-UI s1 Mus musculus cDNA, 3 end/clone = UI-M-BH2 1-apg-d-05-0-UI/ | 257.7 | P | 319.1 | P | 254.5 | P | 189.2 |
| | | clone_end = 3/gb = AW123210/gi = 6098780/ug = Mm 3369/len = 468/STRA for | | | | | | | |
| 3 | 97138_at | Cluster Incl C77335 C773355 Mus musculus cDNA, 3 end/clone = J0029D01/clone_end = 3/gb = C77335/gi = 2517665/ | 173.5 | P | 153.6 | P | 110.8 | P | 127.8 |
| | | ug = Mm 34933/len = 582/STRA = rev | | | | | | | |
| 3 | 97160_at | Cluster Incl X04017: Mouse mRNA for cysteine-rich glycoprotein SPARC/cds = (89,997)/gb = X04017/gi = 54168/ | 2971.9 | P | 4106.1 | P | 3884 | P | 3557 |
| | | ug = Mm 35439/len = 2079/STRA = for | | | | | | | |
| 3 | 97181_f_at | Cluster Incl M10062 Mouse IgE-binding factor mRNA, complete cds/cds = UNKNOWN/gb = M10062/gi = 192166/ | 3783.1 | P | 3702.5 | P | 3858.6 | P | 3548.5 |
| | | ug = Mm 35835/len = 3305/STRA = for | | | | | | | |
| 3 | 97759_at | Cluster Incl U09383 Mus musculus calcium-activated potassium channel mRNA, complete cds/cds = (820,4374)/ | 126.4 | P | 130.8 | P | 137 | P | 137.3 |
| | | gb = U09383/gi = 487796/ug = Mm 4123/len = 5001/STRA = for | | | | | | | |
| 3 | 98278_at | Cluster Incl AB019280 Mus musculus mRNA for sprouty-4, complete cds/cds = (215,1117)/gb = AB019280/gi = 4850325/ | 669.5 | P | 388.2 | P | 728.6 | P | 642.9 |
| | | ug = Mm 42038/len = 1131/STRA = for | | | | | | | |
| 3 | 99405_at | Cluster Incl U30241 Mus musculus anti-DNA antibody Ig kappa chain mRNA, v-j region, hybndoma 84.15, partial cds/ | 210.7 | P | 217.4 | P | 233.4 | P | 201.6 |
| | | cds = (0,298)/gb = U30241/gi = 896180/ug = Mm 57033/len = 299/STRA for | | | | | | | |
| 3 | 99823_r_at | Cluster Incl C79676 C79676 Mus musculus cDNA, 3 end/clone = J0070C12/clone_end = 3/gb = C79676/gi = 2520006/ | 299.8 | P | 313.2 | P | 332.6 | P | 387.9 |
| | | ug = Mm 5866/len = 532/STRA = rev | | | | | | | |
| 3 | 99875_at | Cluster Incl Z32675 Hairless/cds = (377,3925)/gb = Z32675/gi 531706/ug = Mm 7598/len = 4017/STRA = for | 405.1 | P | 487.8 | P | 475.5 | P | 505.2 |
| 3 | 99896_at | Cluster Incl U08354 Melanocortin 5 receptor/cds = (28,1146)/gb = U08354/gi = 522165/ug = Mm 8003/len 1180/STRA = for | 281.8 | P | 482.3 | P | 350.1 | P | 268.1 |
| 3 | 100293_at | Cluster Incl AA268823 vb01d09 r1 Mus musculus cDNA, 5 end/clone = IMAGE-747665/clone_end = 5/gb = AA268823/ | 244.4 | P | 255 | P | 304.8 | P | 175.5 |
| | | gi = 1905739/ug = Mm 86378/len = 435/STRA = for | | | | | | | |
| 3 | 96963_s_at | Cluster Incl L14553 Mus musculus Ig light chain V-region CC49 rearranged gene, exons 1 and 2/cds = (0,399)/gb = L14553/ | 160.9 | P | 168.2 | P | 212.8 | P | 152.8 |
| | | gi = 293639/ug = Mm 88797/len = 400/STRA = for | | | | | | | |
| 3 | 97058_f_at | Cluster Incl AW208630: uo62a10.x1 Mus musculus cDNA, 3 end/clone = IMAGE-2647098/clone_end = 3/gb = AW208630/ | 582.9 | P | 620.7 | P | 425.7 | P | 564.8 |
| | | gi = 6514570/ug = Mm 89273/len = 197/STRA = rev | | | | | | | |
| 3 | 97659_f_at | Cluster Incl X04725.Insulin I/cds = (0,326)/gb = X04725/gi = 52712/ug = Mm 89940/len = 327/STRA = for | 1040.3 | P | 967.3 | P | 995.9 | P | 974.6 |
| 3 | 98245_at | Cluster Incl C77789 C77789 Mus musculus cDNA, 3 end/clone = J0037F03/clone_end = 3/gb = C77789/gi = 2518119/ | 277.9 | P | 286.3 | P | 646.2 | P | 407.1 |
| | | ug = Mm 91361/len = 623/STRA = rev | | | | | | | |
| 3 | 100225_f_at | Cluster Incl AA09481 EST01210 Mus musculus cDNA, 3 end/clone = C0007B09/clone_end = 3/gb = AA409481/ | 229.3 | P | 282.6 | P | 385.6 | P | 339.3 |
| | | gi = 2066125/ug = Mm.98618/len = 254/STRA = rev | | | | | | | |
| 3 | 101152_at | Cluster Incl Z18278 5-hydroxytryptamine (serotonin) receptor 5A/cds = (502,1575)/gb = Z18278/gi = 49758/ug = Mm 4835/ | 382.5 | P | 383.7 | P | 403.6 | P | 391.3 |
| | | len = 1680/STRA = for | | | | | | | |
| 3 | 101186_at | Cluster Incl X65588 M musculus mp41 mRNA/cds = (15,416)/gb = X65588/gi = 1524150/ug = Mm 5118/len = 2857/STRA = for | 241.9 | P | 335.7 | P | 261.5 | P | 244.6 |
| 3 | 101217_at | cluster Incl D18885 MUSGS01047 Mus musculus cDNA, 3 end/clone = mc0128/clone_end = 3/gb = D18865/gi = 1089498/ | 213.9 | P | 187.6 | P | 206 | P | 249.9 |
| | | ug = Mm.5344/len = 314/STRA = rev | | | | | | | |
| 3 | 92572_at | Cluster Incl AI509617 vx14h07.y1 Mus musculus cDNA, 5 end/clone = IMAGE-1264477/clone_end = 5/gb = AI509617/ | 97.3 | P | 148.8 | A | 182.5 | P | 125.5 |
| | | gi = 4408522/ug = Mm 10331/len = 448/STRA = rev | | | | | | | |
| 3 | 92887_at | Cluster Incl AF004106 Mus musculus unknown mRNA, complete cds/cds = (234,968)/gb = AF004106/gi = 2183320/ | 1533.6 | P | 1550.3 | P | 1601 | P | 1400.9 |
| | | ug = Mm 1457/len = 1270/STRA = for | | | | | | | |
| 3 | 93020_at | Cluster Incl AF051347 Mus musculus REX-3 mRNA, complete cds/cds = (228,578)/gb = AF051347/gi = 3510642/ | 30.3 | A | 84.9 | P | 117.7 | P | 82.9 |
| | | ug = Mm.14768/len = 838/STRA = for | | | | | | | |
| 3 | 93821_at | Cluster Incl AW046101.UI-M-BH1-alf-b-06-0-UI s1 Mus musculus cDNA, 3 end/clone = UI-M-BH1-alf-b-06-0-UI/ | 214 | P | 161.9 | P | 211.7 | P | 214.1 |
| | | clone_end = 3/gb = AW046101/gi = 5906630/ug = Mm 21514/len = 261/STRA = rev | | | | | | | |
| 3 | 94877_at | Cluster Incl AI848446 UI-M-AH1-ags-f-03-0-UI s1 Mus musculus cDNA, 3 end/clone = UI-M-AH1-ags-f-03-0-UI/ | 106.4 | P | 99.8 | P | 101.8 | P | 51.2 |
| | | clone_end = 3/gb = AI848446/gi = 5492352/ug = Mm.23839/len = 514/STRA = for | | | | | | | |
| 3 | 94472_at | Cluster Incl AW123216 UI-M-BH2.1-apg-d-12-0-UI s1 Mus musculus cDNA, 3 end/clone = UI-M-BH2 1-apg-d-12-0-UI/ | 254.2 | P | 255.7 | P | 216.5 | P | 263.3 |
| | | clone_end = 3/gb = AW123216/gi = 6098786/ug = Mm 28852/len = 287/STRA = for | | | | | | | |
| 3 | 97482_at | Cluster Incl AA832565: vw43b11.r1 Mus musculus cDNA, 5 end/clone = IMAGE-1246557/clone_end = 5/gb = AA832565/ | 451.1 | P | 387.8 | P | 531.2 | P | 546.6 |
| | | gi = 2906293/ug = Mm.30762/len = 478/STRA = for | | | | | | | |
| 3 | 97536_at | Cluster Incl AW046650: UI-M-BH1-ald-e-10-0-UI s1 Mus musculus cDNA, 3 end/clone = UI-M-BH1-aid-e-10-0-UI/ | 393.6 | P | 421.6 | P | 481.3 | P | 320.8 |
| | | clone_end = 3/gb = AW046650/gi = 5907179/ug = Mm3308/len = 336/STRA = for | | | | | | | |
| 3 | 100997_at | Cluster Incl L22550: Desmin/cds = UNKNOWN/gb = gb 32 L22550/gi = 348971/ug = Mm 6712/len = 1497/STRA = for | 508.7 | A | 498.4 | P | 502.9 | P | 548.1 |
| 3 | 103616_at | Cluster Incl AI181132.ub95c0.r1 Mus musculus cDNA, 5 end/clone = IMAGE-1396224/clone_end = 5/gb = AI181132/ | 197.1 | A | 304 | P | 316.9 | P | 459.6 |
| | | gi = 3731770/ug = Mm20240/len = 499/STRA = for | | | | | | | |
| 3 | 104515_at | Cluster Incl AI286818 uc55b02 r1 Mus musculas cDNA, 5 end/clone = IMAGE-1429515/clone_= 5/gb = AI286818/ | 147.5 | P | 129.5 | P | 196.2 | P | 108.9 |
| | | gi = 3926571/ug = Mm.22765/len = 417/STRA = for | | | | | | | |
| 3 | 104529_at | Cluster Incl U21960: Calcium modulating ligand/cds = (34,918)/gb = U21960/gi = 726183/ug = Mm 2313/len = 1331/STRA = for | 105.2 | P | 186.1 | P | 93.6 | P | 124.4 |
| 3 | 98461_at | Cluster Incl AI987835 um07f07.x1 Mus musculus cDNA, 3 end/clone = IMAGE-2182405/clone _end = 3/gb = AI987935/ | 269.1 | P | 293.7 | P | 235.5 | A | 245 |
| | | gi = 5819629/ug = Mm 34789/len = 623/STRA = for | | | | | | | |
| 3 | 100429_at | Cluster Incl U89155 untitled/cds = (0,149)/gb = U89155/gi = 2707592/ug = Mm 4730/len = 150/STRA = for | 108.6 | P | 165.8 | P | 197.8 | P | 83.3 |
| 3 | 100935_at | Cluster Incl U43548 Mus musculus trasncription factor like protein 4 TCFL4 mRNA, partial cds/cds(0,892)/gb = U43548/ | 384.9 | P | 647 | P | 516 | P | 580.1 |
| | | gi = 1927189/ug = Mm.628/len = 1963/STRA = for | | | | | | | |
| 3 | 100962_at | Cluster Incl U47543 Ngfi-A binding protein 2/cds = (220,1797)/gb = U47543/gi = 197730/ug = Mm 6549/len = 2539 | 90.8 | P | 147.6 | P | 186.7 | P | 64.4 |
| | | STRA = for | | | | | | | |
| 3 | 101359_at | Cluster Incl U43541 Lamin,beta 2/cds = (0,5399)/gb = U43541/gi = 1244719/ug = Mm 68867/len = 5400/STRA = for | 107.4 | P | 169.3 | P | 239.8 | P | 145.3 |
| 3 | 101861_at | Cluster lncl AF031919 Sarcoglycan, epsilon/cds = (44,1285)/gb = AF031919/gi = 3057139/ug = Mm.8739/len = 1548 | 297.9 | P | 402.7 | P | 435.7 | P | 366.2 |
| 3 | 101913_at | Cluster Incl AW214298 uo39g06.x1 Mus musculus cDNA, 3 end/clone = IMAGE-2644954/clone_end = 3/gb = AW214298/ | 207.8 | M | 269.6 | P | 219.7 | P | 218.4 |
| | | gi = 6520787/ug = Mm.90654/len = 172/STRA = for | | | | | | | |
| 3 | 102878_at | Cluster Incl U12135 RAD52 homolog, (S cerevisiae/cds = (114,1373)/gb = U12135/gi = 602158/ug = Mm 149/len = 1664/ | 388.6 | A | 296.3 | P | 406.3 | P | 283.8 |
| | | STRA = for | | | | | | | |
| 3 | 103547_at | Cluster Incl AI837116 UI-M-AK0-adc-e-09-0-UI a1 Mus musculus cDNA, 3 end/clone = UI-M-AK0-adc-e-09-0-UI/ | 351.9 | P | 281.4 | P | 361.9 | P | 266.9 |
| | | clone_end = 3/gb = AI837116/gi = 5471329/ug = Mm 23723/len = 323/STRA = rev | | | | | | | |
| 3 | 103927_at | Cluster Incl AW212672 uo10c08 x1 Mus musculus cDNA, 3 end/clone = IMAGE-2599118/clone_end = 3/gb = AW212672/ | 52.1 | A | 162.3 | P | 224.1 | P | 130.7 |
| | | gi = 6518785/ug = Mm 25086/len = 154/STRA = for | | | | | | | |
| 3 | 104164_at | Cluster Incl AI181257 ub94b03 r1 Mus musculus cDNA, 5 end/clone = IMAGE-1396109/clone_end = 5/gb = AI181257/ | 40.5 | P | 173.7 | P | 258.2 | P | 211.4 |
| | | gi = 3731895/ug = Mm.27139/len = 510/STRA = for | | | | | | | |
| 3 | 104252_at | Cluster Incl AW123823 UI-M-BH2 1-apm-g-07-0-UI s1 Mus musculus cDNA, 3 end/clone = UI-M-BH2 1-apm-g-07-0-UI/ | 222.2 | P | 176.3 | P | 189.9 | P | 183.8 |
| | | clone_end = 3/gb = AW123823/gi = 6099353/ug = Mm 28362/len = 386/STRA = for | | | | | | | |
| 3 | 104265_at | Cluster Incl X70842 Kinase insert domain protein receptor/cds = (285,4388)/gb = X70842/gi = 57923/ug = Mm.285/len = 5470/ | 340.7 | A | 408.4 | P | 474.8 | P | 453.6 |
| | | STRA = for | | | | | | | |
| 3 | 92269_r_at | Cluster Incl AI836610 UI-M-AP0-abf-g-08-0-UI.s1 Mus musculus cDNA, 3 end/clone = UI-M-AP0-abf-g-08-0-UI/ | 88.9 | P | 82.2 | P | 95.2 | P | 66 |
| | | clone_end = 3/gb = AI836610/gi = 5470823/ug = Mm.3590/len = 130/STRA = for | | | | | | | |
| 3 | 92518_at | Cluster Incl Y09535 M musculus mRNA for neogenin protein/cds = (180,4661)/gb = Y09535/gi = 1881476/ug = Mm 42249/ | 483.7 | P | 532.8 | P | 488.3 | P | 436.1 |
| | | len = 5414/STRA = for | | | | | | | |
| 3 | 92691_at | Cluster Incl AW048155 UI-M-BH1-alq-h-08-0-UI s1 Mus musculus cDNA, 3 end/clone = UI-M-BH1-alq-h-08-0-UI/ | 599.4 | P | 539.2 | P | 703 | P | 586.2 |
| | | clone_end = 3/gb = AW048155/gi = 5908684/ug = Mm 45608/len = 425/STRA = for | | | | | | | |
| 3 | 92942_at | Cluster Incl AA138192: mr05c12 r1 Mus musculus cDNA, 5 end/clone = IMAGE-596566/clone_end = 5/gb = AA138192/ | 521 | P | 504.9 | M | 455.2 | P | 486.4 |
| | | gi = 1700519/ug = Mm 492/len = 468/STRA = rev | | | | | | | |
| 3 | 93191_at | Cluster Incl AW061328 UI-M-BH1-anw-f-01-0-UI s1 Mus musculus cDNA, 3 end/clone = UI-M-BH1-anw-f-01-0-UI/ | 163.8 | A | 186.7 | P | 233.6 | P | 148.7 |
| | | clone_end = 3/gb = AW061328/gi = 6009079/ug = Mm 5572/len = 461/STRA = rev | | | | | | | |
| 3 | 93392_at | Cluster Incl AB010742: Uncoupling protein 3, mitochondrial/cds = (197,1123)/gb = AB010742/gi = 3062840/ug = Mm.6254/ | 237.4 | M | 275.4 | P | 276.6 | P | 217.1 |
| | | len = 2460/STRA = rev | | | | | | | |
| 3 | 93690_at | Cluster Incl AF022072 Growth factor receptor bound protein 10/cds = (308,2098)/gb = AF022072/gi = 2465448/ug = Mm 8432/ | 409.5 | P | 326.9 | M | 495.2 | P | 411.3 |
| | | len = 2176/STRA = for | | | | | | | |
| 3 | 94174_at | Cluster Incl AF006071 Mus musculus alpha-catenin related protein (ACRP) mRNA, complete cds/cds = (84,2279)/ | 94.3 | P | 71.6 | P | 13.6 | P | 60.2 |
| | | gb = AF006071/gi = 3309106/ug = Mm.12878/len = 2292/STRA = for | | | | | | | |
| 3 | 94790_at | Cluster Incl AA681807: vu65f09 r1 Mus musculus cDNA, 5 end/clone = IMAGE-1196297/clone_end = 5/gb = AA681807/ | 132.1 | A | 187 | P | 184.5 | P | 77.8 |
| | | gi = 2663947/ug = Mm 17221/len = 318/STRA = rev | | | | | | | |
| 3 | 96541_at | Cluster Incl AI838636 UI-M-AO0-aca-a-10-0-UI.s1 Mus musculus cDNA, 3 end/clone = UI-M-AO0-aca-a-10-0-UI/ | 197 | P | 183.6 | P | 146.4 | P | 147.3 |
| | | clone_end = 3/gb = AI838636/gi = 5472849/ug = Mm 27795/len = 369/STRA = rev | | | | | | | |
| 3 | 98869_g_at | Cluster Incl L31532 B-cell leukemia/lymphoma 2/cds = (1197,1796)/gb = L31532/gi = 468337/ug = Mm.5155/len = 1897/ | 275.4 | P | 281.8 | P | 305.1 | P | 285.2 |
| | | STRA = for | | | | | | | |
| 3 | 99813_g_at | Cluster Incl X92523 Calpain 3/cds = (0,2465)/gb = X92523/gi = 1246778/ug = Mm 57235/len = 2466/STRA = for | 39.5 | P | 63.7 | P | 77.5 | P | 33.5 |
| 3 | 99825_at | Cluster Incl AA881307: vx11d09.r1 Mus musculus cDNA, 5 end/clone = IMAGE-1264145/clone_end = 5/gb = AA881307/ | 295.1 | P | 252.5 | A | 303.6 | P | 252.8 |
| | | gi = 2990617/ug = Mm 5980/len = 511/STRA = rev | | | | | | | |
| 3 | 99842_at | Cluster Incl AB000636: Procollagen, type XIX, alpha 1/cds = (98,3508)/gb = AB000636/gi = 2627208/ug = Mm 6252/len = 4205/ | 134.6 | P | 114.9 | P | 128.9 | P | 100.7 |
| | | STRA = for | | | | | | | |
| 3 | 100366_at | Cluster Incl AB019600: Mus musculus mRNA for caspase9, complete cds/cds = (0,1364)/gb = AB019600/gi = 6440941/ | 328.8 | P | 322.1 | P | 376.2 | P | 349.3 |
| | | ug = Mm.88829/len = 1365/STRA = for | | | | | | | |
| 3 | 101716_at | Cluster Incl AF017280 Mus musculus ribonuclease 5 precursor (mR-5) gene, complete cds/cds = (0,467)/gb = AF017260/ | 84.6 | P | 78.7 | P | 71.9 | P | 63.2 |
| | | gi = 2407241/ug = Mm 57025/len = 468/STRA = for | | | | | | | |
| 3 | 101830_at | Cluster Incl X83934: Ryanodine receptor 3/ods = (0,866)/gb = X83934/gi = 639816/ug = Mm 57201/len = 867/STRA = for | 189.4 | P | 201.4 | P | 229.9 | P | 175.5 |
| 3 | 101633_at | Cluster Incl X16678: Immunoglobulin kappa chain variable 20 (V20 family)/cds = (0,383)/gb = X16678/gi = 55388/ | 449.9 | P | 613.3 | P | 795.8 | P | 712.7 |
| | | ug = Mm 6294/len = 384/STRA = for | | | | | | | |
| 3 | 99187_f_at | Cluster Incl AI835662: UI-M-AJ0-aau-e-03-0-UI s1 Mus musculus cDNA, 3 end/clone = UI-M-AJ0-aau-e-03-0-UI/ | 437 | P | 482 | P | 436.5 | P | 344.5 |
| | | clone_end = 3/gb = AI835662/gi = 5469875/ug = Mm 41890/len = 268/STRA = for | | | | | | | |
| 3 | 103578 _at | Cluster Incl AJ006972: Mus musculus mRNA for TOM1 protein/cds = (67,1545)/gb = AJ006972/gi = 3319987/ug = Mm.1967/ | 247.3 | P | 263.9 | P | 292 | P | 346.9 |
| | | len = 2253/STRA = for | | | | | | | |
| 3 | 104591_g_at | Cluster Incl 13171: Myocyte enhancer factor 2C/cds = UNKNOWN/gb = L13171/gi = 293728/ug = Mm.24001/len = 1656/ | 61.8 | P | 127.3 | P | 61.5 | A | 72.2 |
| | | STRA = for | | | | | | | |
| 3 | 97944_f_at | Cluster Incl AF099808: Mus musculus T cell receptor alpha chain variable region (Tcra-V11 1d) gene, allele S1, partial cds/ | 367.8 | M | 378.7 | M | 414.4 | P | 349.3 |
| | | cds = (0,233)/gb = AF099808/gi = 4323544/ug = Mm 30295/len = 234/STRA = for | | | | | | | |
| 3 | 101893_r_at | Cluster Incl z67963 Fussed toes/cds = (213,1091)/gb = Z67963/gi = 1067144/ug = Mm 88777/len = 2173/STRA = for | 32.1 | P | 34.4 | P | 30.1 | P | 28.3 |
| 3 | 103302_r_at | Cluster Incl AA866668 ud07a10 r1 Mus musculus cDNA, 5 end/clone = IMAGE-1424426/clone_end = 5/gb = AA866668/ | 274.5 | P | 259 | A | 293.5 | A | 296.5 |
| | | gi = 2962113/ug = Mm 21097/len = 537/STRA = for | | | | | | | |
| 3 | 92401_at | Cluster Incl U27195 Leukotnene C4 synthase/cds = (36,488)/gb = U27195/gi = 973221/ug = Mm.4088/len = 588/STRA = for | 202.7 | P | 196.6 | P | 167 | P | 137.2 |
| 3 | 92519_at | Cluster Incl X74616 Phosphorylase kinase alpha 1/cds = (218,3943)/gb = X74616/gi= 436123/ug = Mm 42254/len = 4130/ | 258.3 | P | 226.1 | P | 328.7 | A | 232.1 |
| | | STRA = for | | | | | | | |
| 3 | 94715_at | Cluster Incl K02588 Cytochrome P450, 1a1, aromatic compound inducible/cds = (109,1674)/gb = K02588/gi = 192885/ | 113.4 | P | 198.2 | P | 198.1 | M | 227.8 |
| | | ug = Mm 14089/len = 2619/STRA = for | | | | | | | |
| 3 | 97743_at | Cluster Incl AW047461 UI-M-BH1-all-e-12-0-UI s1 Mus musculus cDNA, 3 end/clone = UI-M-BH1-all-e-12-0-UI/ | 1470.4 | P | 1600.9 | A | 1601.5 | P | 1484.5 |
| | | clone_end = 3/gb = AW047461/gi = 5907990/ug = Mm 4016/len = 254/STR = rev | | | | | | | |
| 3 | 98847₁₃ at | Cluster Incl U52826 Mus musculus syndecan-3 mRNA, complete cds/cds = (34,1362)/gb = U52826/gi = 1399582/ | 349.5 | P | 354.9 | P | 337 | P | 317.2 |
| | | ug = Mm.5059/len = 1495/STRA = for | | | | | | | |
| 3 | 97002_f_at | Cluster Incl AJ133427 Mus musculus or37d pseudogene/cds = (0,962)/gb = AJ133427/gi = 5869921/ug = Mm 88844/len = 963/ | 375.5 | P | 324.4 | P | 278.9 | P | 366.7 |
| | | STRA = for | | | | | | | |
| 3 | 102113_f_at | Cluster Incl AI324061 mt75g06 x1 Mus musculus cDNA, 3 end/clone = IMAGE-635770/clone_end = 3/gb = AI324061/ | 105.7 | A | 323.5 | P | 307.1 | P | 252 |
| | | gi = 4058490/ug = Mm 42606/len = 258/STRa = rev | | | | | | | |
| 3 | 92786_at | Cluster Incl AI626942.vs77b11.x1 Mus musculus cDNA, 3 end/clone = IMAGE-1152285/clone_end= 3/gb= AI626942/ | 152.5 | P | 174.8 | P | 144.5 | A | 109.5 |
| | | gi = 4663742/ug = Mm 12454/len = 306/STRA = for | | | | | | | |
| 3 | 93359_at | Cluster Incl AI839743 UI-M-AP0-abe-e-06-0-UI s2 Mus musculus cDNA, 3 end/clone = UI-m-APO-abe-e-06-0-UI/ | 131.1 | M | 122.3 | P | 125.4 | P | 99.2 |
| | | clone_end = 3/gb = AI839743/gi = 5473956/ug = Mm 1893/len = 456/STRA = for | | | | | | | |
| 3 | 93592_at | Cluster Incl X82648 M musculus mRNA for apolipoprotein D/cds = (89,658)/gb = X82648/gi = 575656/ug = Mm 2082/len = 903/ | 143.1 | A | 136.8 | P | 141 | P | 163.7 |
| | | STRA = for | | | | | | | |
| 3 | 94857_at | Cluster Incl U10420 N-methylpurine-DNA glycosylase/cds = (29,1030)/gb = U10420/gi = 500754/ug = Mm 2358/len = 1061/ | 87 | P | 103.8 | A | 88.3 | P | 118.6 |
| | | STRA = for | | | | | | | |
| 3 | 102070_at | Cluster Incl AW212495 uo89g01 x1 Mus musculus cDNA, 3 end/clone = IMAGE-2649744/clone_end = 3/gb = AW212495/ | 685.4 | A | 737.2 | A | 706.6 | P | 462.9 |
| | | gi = 6518582/ug = Mm.106/len = 429/STRA = for | | | | | | | |
| 3 | 102312_r_at | Cluster Incl AW046728 UI-M-BH1-alt-g-05-0-UI s1 Mus musculus cDNA, 3 end/clone = UI-M-BH1-alt-g-05-0-UI/ | 688.6 | P | 794.9 | P | 646 | P | 608 |
| | | clone_end = 3/gb = AW046728/gi = 5907297/ug = Mm.10641/len = 302/STRA = for | | | | | | | |
| 3 | 102314_at | Cluster Incl M23383 Solute carrier family 2 (facilitated glucose transporter), member 4/cds = (153,1685)/gb = M23383/ | 124.4 | M | 217.4 | P | 182.2 | M | 108 |
| | | gi = 193706/ug = Mm 10661/len = 2521/STRA = for | | | | | | | |
| 3 | 103369_at | Cluster Incl AW049142 UI-M-BH1-amq-b-11-0-UI a1 Mus musculus cDNA, 3 end/clone = UI-M-BH1-amq-b-11-0-UI/ | 367.6 | A | 414.5 | A | 309.7 | A | 408.3 |
| | | clone_end = 3/gb = AW049142/gi = 5909671/ug = Mm.18538/len = 446/STRA = rev | | | | | | | |
| 3 | 103377_at | Cluster Incl AW259788 um81b12 y1 Mus musculus cDNA, 5 end/clone = IMAGE-2317727/clone_end = 5/gb = AW259788/ | 122.2 | P | 139.7 | P | 134.1 | A | 131.1 |
| | | gi = 6632769/ug = Mm.18600/len = 507/STRA = for | | | | | | | |
| 3 | 103838_at | Cluster Incl AB010144 Mus musculus gene for mitsugumin29/cds = (183,977)/gb = AB010144/gi = 3761106/ug = Mm 20942/ | 339.1 | A | 383.3 | A | 398.7 | P | 398.3 |
| | | len = 3248/STRA = for | | | | | | | |
| 3 | 94977_at | Cluster Incl X15373 Inositol 1,4,5-triphosphate receptor 1/cds = (328,8577)/gb = X15373/gi = 53568/ug = Mm 2726/len = 9841/ | −11.2 | A | 38.2 | P | 41.2 | A | 52.3 |
| | | STRA = for | | | | | | | |
| 3 | 97403_at | Cluster Incl AW045743 UI-M-BH1-akq-h-05-0-UI s1 Mus musculus cDNA, 3 end/clone = UI-M-BH1-akq-h-05-0-UI/ | 334.2 | P | 319.9 | P | 227 | A | 232.9 |
| | | clone_end = 3/gb = AW045743/gi = 5906272/ug = Mm 29900/len = 257/STRA = for | | | | | | | |
| 3 | 98513_at | Cluster Incl AI851821: UI-M-BH0-ais-d-03-0-UI s1 Mus musculus cDNA, 3 end/clone = UI-M-BH0-ais-d-03-0-UI/ | 411.6 | M | 366.8 | M | 457.4 | P | 398.5 |
| | | clone_end = 3/gb = AI851821/gi = 5495727/ug = Mm 3600/len = 420/STRA = for | | | | | | | |
| 3 | 99050_at | Cluster Incl U36579 Caveolin 3/cds = (121,576)/gb = U36579/gi = 1155228/ug = Mm 3924/len = 1000/STRA = for | 195.1 | A | 431.4 | P | 456.4 | P | 392.3 |
| 3 | 102639_at | Cluster Incl AB011451 Mus musculus mRNA for N-acetylgucosamine-6-O-sulfotransferase, complete cds/cds = (469,1920)/ | 101 | P | 104.3 | A | 111.8 | P | 64.3 |
| | | gb = AB011451/gi = 3402294/ug = Mm.12968/len = 2150/STRA = for | | | | | | | |
| 3 | 103212_at | Cluster Incl AI314784 uj35c08 x1 Mus musculus cDNA, 3 end/clone = IMAGE-1921934/clone_end = 3/gb = AI314784/ | 227.7 | A | 184.2 | P | 208.1 | P | 143.8 |
| | | gi = 4030051/ug = Mm 19726/len = 494/STRA = for | | | | | | | |
| 3 | 104678_at | Cluster Incl U19860 Growth arrest specific 7/cds = (130,1395)/gb = U19860/gi = 1154621/ug = Mm.3257/len = 2888/ | 116.9 | P | 119.9 | M | 169.6 | P | 139.5 |
| | | STRA = for | | | | | | | |
| 3 | 92323_at | Cluster Incl Y13439 Mus musculus mRNA for stress activated protein kinase-3/cds = (167,1270)/gb = Y13439/gi = 2181949/ | 175.2 | A | 195.5 | P | 225 | A | 120 |
| | | ug = Mm 38343/len = 1553/STRA = for | | | | | | | |
| 3 | 92387_at | Cluster Incl L18868 Thromboxane A synthase 1, platelet/cds = (38,1839)/gb = L18868/gi = 1763715/ug = Mm.4054/len = 1847/ | 598.7 | P | 556.3 | P | 706.8 | P | 563.3 |
| | | STRA = rev | | | | | | | |
| 3 | 92535_at | Cluster Incl L12147 Earty B-cell factor/cds = (45,1820)/gb = L12147/gi = 309208/ug = Mm 4366/len = 2248/STRA = for | 145.5 | P | 121.5 | P | 169.4 | P | 173.4 |
| 3 | 93893_f_at | Cluster Incl U56404: Mus musculus strain C57BL/8 natural killer cell receptor Ly-49C mRNA, complete cds/cds = (49,849)/ | 42.5 | A | 88.6 | P | 64.1 | M | 68.8 |
| | | gb = U56404/gi = 1336030/ug = Mm 89187/len = 907/STRA = for | | | | | | | |
| 3 | 94757_at | Cluster Incl M60493 Cystic fibrosis transmembrane conductance regulator homolog/cds = (138,4568)/gb = M60493/ | 103.9 | P | 98.7 | P | 80.6 | P | 74.7 |
| | | gi = 192831/ug = Mm.15621/len = 6304/STRA = for | | | | | | | |
| 3 | 95358_at | Cluster Incl AI843864 UI-M-AK1-aeu-c-11-0-UI s1 Mus musculus cDNA, 3 end/clone = UI-M-AK1-aeu-c-11-0-UI/ | 303.3 | P | 342.8 | P | 434.9 | P | 253.7 |
| | | clone_end = 3/gb = AI843864/gi = 5478077/ug = Mm 21518/len = 341/STRA = for | | | | | | | |
| 3 | 97158_at | Cluster Incl AI840785 UI-M-AM0-adn-h-05-0-UI.s1 Mus musculus cDNA, 3 end/clone = UI-M-AM0-adn-h-05-0-UI/ | 218.7 | A | 314.3 | P | 265.9 | P | 274.5 |
| | | clone_end = 3/gb = AI840785/gi = 5474998/ug = Mm.35235/len = 324/STRA = rev | | | | | | | |
| 3 | 97761_f_at | Cluster Incl U10094 Killer cell lectin-like receptor, subfamily A, member 7/cds = (70,873)/gb = U10094/gi = 533495/ | 263.8 | P | 256.5 | P | 309.7 | P | 189.6 |
| | | ug = Mm 4131/len = 1075/STRA = for | | | | | | | |
| 3 | 98782_at | Cluster Incl D38613: Mouse 921-L mRNA for presynaptic protein, complete cds/cds = (84,488)/gb = D38613/gi = 1041029/ | 396.9 | A | 411 | P | 385.5 | P | 415.5 |
| | | ug = Mm.4705/len = 1080/STRA = for | | | | | | | |
| 3 | 99435_at | Cluster Incl Z22703 Fibroblast growth factor 7/cds = (0,584)/gb = Z22703/gi = 297755/ug = Mm 57177/len = 585/STRA = for | 154.1 | M | 214.8 | A | 230.3 | P | 154.3 |
| 3 | 101684_r_at | Cluster Incl X67863 M musculus T2 mRNA/cds = (174,731)/gb = X67863/gi = 296381/ug = Mm 35868/len = 1263/STRA = for | 619.3 | A | 656.2 | A | 589.3 | P | 635.1 |
| 3 | 93294_at | Cluster Incl M70642: Fibroblast Inducible secreted protein/cds = (137,1183)/gb = M70642/gi = 193315/ug = Mm.1810/ | 141.4 | A | 80 | A | 161.6 | P | 86.3 |
| | | len = 2267/STRA = for | | | | | | | |
| 3 | 93532_at | Cluster Incl J04992 Troponin I, skeletal, fast 2/cds = (23,571)/gb = J04992/gi = 202164/ug = Mm 1984/len = 667/STRA = for | 371.4 | A | 694.5 | P | 520.7 | A | 645.6 |
| 3 | 95401_at | Cluster Incl AW050156: UI-M-BH1-ano-g-04-0-UI s1 Mus musculus cDNA, 3 end/clone = UI-M-BH1-ano-g-04-0-UI/ | 765.4 | A | 726.8 | P | 744 | P | 1038.4 |
| | | clone_end = 3/gb = AW050156/gi = 5910685/ug = Mm.24195/len = 443/STRA = for | | | | | | | |
| 3 | 98106_at | Cluster Incl U69898 Mus musculus translocase of inner mitochondrial membrane (Tim44) mRNA, nuclear gene encoding | 636.9 | P | 594.4 | P | 601.1 | A | 527.1 |
| | | mitochondrial protein, complete cds/cds = (8,1366)/gb = U69898/gi = 2351409/ug = Mm.34791/len = 1764/STRA = for | | | | | | | |
| 3 | 103815_at | Cluster Incl AI852311 UI-M-BH0-aje-g-04-0-UI s1 Mus musculus cDNA, 3 end/clone = UI-M-BH0-aje-g-04-0-UI/ | 146.7 | A | 233.5 | P | 189.3 | A | 207.9 |
| | | clone_end = 3/gb = AI852311/gi = 5496217/ug = Mm.20901/len = 471/STRA = for | | | | | | | |
| 3 | 94442_s_at | Cluster Incl AF030001: Mus musculus major histocompatibility locus class III region-butyrophilin-like protein gene, partial | 316.7 | P | 238.8 | A | 339.2 | P | 287.8 |
| | | cds, Notch4, PBX2, RAGE, lysophatidic acid acyl transferase-alpha, palmitoyl-protein thioesterase 2 (PPT2), CREB-RP, | | | | | | | |
| | | and tenascin X/STRA = for | | | | | | | |
| 3 | 97336_at | Cluster Incl AJ131851: Mus musculus mRNA for cathepsin F/cds = (16,1404)/gb = AJ131851/gi = 4826564/ug = Mm.29561 | 180.5 | A | 186.2 | P | 203.1 | A | 221.7 |
| | | len = 1784/STRA = for | | | | | | | |
| 3 | 97379_at | Cluster Incl D42083: Mouse mRNA for fructose 1,6-bisphosphatase, complete cds/cds = (19,1083)/gb = D42083/gi = 575935/ | 111.4 | P | 155.7 | P | 77.2 | A | 102.1 |
| | | ug = Mm 2974/len = 1242/STRA = for | | | | | | | |
| 3 | 98498_at | Cluster Incl D86353: Mouse mRNA for Mch3, complete cds/cds = (52,963)/gb = D86353/gi = 1945545/ug = Mm 35687/ | 63.7 | A | 102.1 | P | 82.4 | P | 118.7 |
| | | len = 964/STRA = for | | | | | | | |
| 3 | 98983_at | Cluster Incl U16163 Mus musculus prolyl 4-hydroxylase alpha(II)-subunit mRNA, complete cds/cds = (150,1783)/ | 120.1 | P | 117.1 | P | 198.6 | P | 48.5 |
| | | gb = U16163/gi = 836899/ug = Mm 3705/len = 2153/STRA = for | | | | | | | |
| 3 | 99044_at | Cluster Incl AI847547 UI-M-AM1-afv-f-02-0-UI.s1 Mus musculus cDNA, 3 end/clone = UI-M-AM1-afv-f-02-0-UI/ | 276 | P | 328.7 | P | 382 | P | 271.7 |
| | | clone_end = 3/gb = AI847547/gi = 5491453/ug = Mm 39099/len = 385/STRA = for | | | | | | | |
| 3 | 99993_at | Cluster Incl U77083 Leucine arylaminopeptidase 1, intestinal/cds = (33,2933)/gb = U77083/gi = 1674500/ug = Mm.4487/ | 241.3 | P | 226.4 | P | 218.4 | P | 174.1 |
| | | len = 3367/STRA = for | | | | | | | |
| 3 | 103959_at | Cluster Incl AI605405 vn78f02.x1 Mus musculus cDNA, 3 end/clone = IMAGE-1038075/clone_end = 3/gb = AI605405/ | 171.9 | P | 192.5 | A | 283.3 | P | 216.6 |
| | | gi = 4614572/ug = Mm 25582/len = 331/STRA = rev | | | | | | | |
| 3 | 93423_at | Cluster Incl AI844772 UI-M-AL1-ahq-f-11-0-UI s1 Mus musculus cDNA, 3 end/clone = UI-M-AL1-ahq-f-11-0-UI/ | 265 | P | 270.8 | P | 191.6 | P | 209.8 |
| | | clone_end = 3/gb = AI844772/gi = 5488678/ug = Mm.6471/len = 297/STRA = rev | | | | | | | |
| 3 | 93618_at | Cluster Incl AF026489: Beta-spectrin 3/cds = (0,47)/gb = AF026489/gi = 2511783/ug = Mm 7363/len = 696/STRA = for | 314 | A | 344.9 | A | 436.6 | P | 361.4 |
| 3 | 93700_at | Cluster Incl AI838022 UI-M-AP0-abi-h-01-0-UI s1 Mus musculus cDNA, 3 end/clone = UI-M-AP0-abl-h-01-0-UI/ | 14.3 | P | −245.4 | A | 23.1 | P | −63.5 |
| | | clone_end = 3/gb = AI838022/gl = 5472235/ug = Mm.8612/len = 226/STRA = for | | | | | | | |
| 3 | 97776_at | Cluster Incl X55674 M musculus mRNA for D2A dopamine receptor/cds = (129,1463)/gb = X55674/gi = 50648/ | 197.3 | P | 126 | A | 182 | M | 136.8 |
| | | ug = Mm 41970/len = 2545/STRA = rev | | | | | | | |
| 3 | 98328_at | Cluster Incl U02602: Thyroid stimulating hormone, receptor/cds = (74,2368)/gb = U02602/gi = 575923/ug = Mm 4254/ | 45.9 | P | 115.5 | P | 79.3 | A | 57.9 |
| | | len = 2512/STRA = for | | | | | | | |
| 3 | 93606_s_at | Cluster Incl AB021966: Mus musculus mRNA for adhesion protein RA175C, complete cds/cds = (0,920)/gb = AB021966 | 105.8 | A | 169.2 | P | 137.2 | M | 220.2 |
| | | gi = 6525254/ug = Mm 20921/len = 921/STRA = for | | | | | | | |
| 3 | 96099_at | Cluster Incl X56502 Casein kinase II, beta subunit/cds = (121,788)/gb = X56502/gi = 50420/ug = Mm 26789/len = 944/ | 766.5 | A | 833.6 | A | 778.3 | P | 873.8 |
| | | STRA = for | | | | | | | |
| 3 | 97242_at | Cluster Incl AI849011 UI-M-AJ1-ahd-g-01-0-UI s1 Mus musculus cDNA, 3 end/clone = UI-M-AJ1-ahd-g-01-0-UI/ | 517.8 | A | 432.2 | A | 768.6 | P | 577.1 |
| | | clone_end = 3/gb = AI849011/gi = 5492917/ug = Mm.2781/len= 467/STRA = for | | | | | | | |
| 3 | 95068_at | Cluster Incl AA763945: vv44b 12.r1 Mus musculus cDNA, 5 end/clone = IMAGE-1225247/clone_end = 5/gb = AA763945/ | 144.7 | P | 182.2 | P | 169.7 | A | 141.9 |
| | | gi = 2811467/ug = Mm.29186/len = 437/STRA = for | | | | | | | |
| 3 | 98452_at | Cluster Incl D88689: Mus musculus mRNA for fit-1, complete cds/cds = (251,4252)/gb = D88689/gi = 2809068/ug = Mm.3464/ | 117.5 | A | 189.2 | A | 241.2 | A | 130.3 |
| | | len = 6275/STRA = for | | | | | | | |
| 3 | 98482_at | Cluster Incl X78936 Parathyroid hormone receptor/cds = (56,1831)/gb = X78936/gi = 474828/ug = Mm 3542/len = 1969/ | 92.4 | A | 119.7 | P | 144 | P | 62.9 |
| | | STRA = for | | | | | | | |
| 3 | 99077_at | Cluster Incl X07750.Thyroid hormone receptor alpha/cds = (0,1232)/gb = X07750/gi = 50387/ug = Mm 4015/len = 1233/ | 240.7 | P | 242.4 | A | 275 | P | 185.9 |
| | | STRA = for | | | | | | | |
| 3 | 103935_at | Cluster Incl AI504474 vo44e10 Mus musculus cDNA, 3 end/clone = IMAGE-1052778/clone_end = 3/gb = AI504474/ | 343 | P | 403.9 | P | 359.1 | P | 335.5 |
| | | gi = 4402325/ug = Mm.25209/len = 265/STRA = rev | | | | | | | |
| 3 | 93919_at | Cluster Incl AB020741: Mus musculus NRK mRNA for NIK-related kinase, complete cds/cds = (247,4614)/gb = AB020741/ | 43.6 | M | 13.3 | A | 38.2 | P | 30 |
| | | gi = 6009518/ug = Mm 89850/len = 5481/STRA = for | | | | | | | |
| 3 | 95988_at | Cluster Incl C79529.C79529 Mus musculus cDNA, 3 end/clone = J0067F08/clone_end = 3/gb = C79529/gi = 2519859/ | 465.6 | A | 473.8 | P | 531.5 | P | 404.5 |
| | | ug = Mm.25039/len = 802/STRA = rev | | | | | | | |
| 3 | 98841_at | Cluster Incl M65287 Activin receptor IIA/cds = (70,1611)/gb = M65287/gi = 191663/ug = Mm 5041/len = 2563/STRA = for | 294.3 | P | 8.8 | A | 267 | A | 243.9 |
| 3 | 99815_at | Cluster Incl D50096 Histamine receptor H 2/cds = (0,1076)/gb = D50096/gi = 1731686/ug = Mm.57243/len = 1077/STRA = for | 243.7 | M | 213.6 | A | 237.4 | P | 236.7 |
| 3 | 101343_at | Cluster Incl AF020263: Mus musculus EWS/FLI1 activated transcript 2 (EAT-2) mRNA, complete cds/cds = (0,398)/ | 106.2 | P | 85.1 | P | 240 | A | 169.4 |
| | | gb = AF020263/gi = 2429353/ug = Mm.57009/len = 399/STRA = for | | | | | | | |
| 3 | 100697_at | Cluster Incl X59358 Paired box gene 3/cds = (297,1736)/gb = X59358/gi = 53591/ug = Mm 1371/len = 2347/STRA = for | 120.3 | P | 115.2 | A | 87.7 | A | 177.4 |
| 3 | AFFX-DapX- | B Subtillis Dap 5 prime | 65 | A | 81.1 | M | 73.2 | P | 57.9 |
| | 5_at | | | | | | | | |
| 3 | 93012-at | Cluster Incl X81464: Translin/cds = (66,752)/gb = X81464/gi = 971555/ug = Mm 14644/len = 991/STRA = for | 112.3 | M | 119.7 | A | 110.8 | P | 112.8 |
| 3 | 96685_at | Cluster Incl AW121774: UI-M-BH2.3-aoe-f-12-0-UI.s1 Mus musculus cDNA, 3 end/clone = UI-M-BH2.3-aoe-f-12-0-UI/ | 405.8 | A | 516.3 | A | 637.4 | A | 399 |
| | | clone_end = 3/gb = AW121774/gi = 6097122/ug = Mm. 27472/len = 196/STRA = for | | | | | | | |
| 3 | 95113_at | Cluster Incl AW124257: UI-M-BH2.1-aph-g-07-0-UI.s1 Mus musculus cDNA, 3 end/clone = UI-M-BH2, 1-aph-g-07-0-UI/ | 31.8 | A | 87.5 | P | 40.2 | M | 39.4 |
| | | clone_end = 3/gb = AW124257/gi = 6099787/ug = Mm 29372/len = 377/STRA = for | | | | | | | |
| 3 | 101034_at | Cluster Incl U07617 Growth factor receptor bound protein 2/cds = (80,733)/gb = U07617/gi = 464004/ug = Mm 6900/ | 251.3 | A | 273.2 | P | 367.8 | P | 271 |
| | | len = 2362/STRA = for | | | | | | | |
| 3 | 99511_at | Cluster Incl X53532 Protein kinase C, beta/cds = (48,2069)/gb = X53532/gi = 53692/ug = Mm 4182/len = 2974/STRA = for | 9.6 | P | 150.5 | A | 62.5 | A | 114.8 |
| 3 | 102958_at | Cluster Incl U82441 Early B-cell factor 2/cds = (23,1750)/gb = U82441/gi = 3757777/ug = Mm 179/len = 1844/STRA = for | 302.8 | M | 293.4 | A | 457.5 | P | 306.8 |
| 3 | 103240_f_at | Cluster Incl AF017258 Mus musculus ribonuclease 3 precursor (mR-3) gene, complete cds/cds = (0,470)/gb = AF017258/ | 180.9 | P | 148.4 | A | 150.8 | A | 159.5 |
| | | gi = 2407237/ug = Mm 20370/len = 471/STRA = for | | | | | | | |
| 3 | 103498_at | Cluster Incl L38613 Mus musculus glucagon receptor mRNA, complete cds/cds = (94,1551)/gb = L38613/gi = 603463/ | 433.5 | A | 364.9 | A | 454.9 | P | 474.2 |
| | | ug = Mm 22329/len = 1944/STRA = for | | | | | | | |
| 3 | 103700_r_at | Cluster Incl AI646838 mt86b06.y1 Mus musculus cDNA, 5 end/clone = IMAGE-636755/clone_end = 5/gb = AI646638/ | 360.2 | A | 299.1 | P | 335.1 | P | 345.4 |
| | | gi = 4725113/ug = Mm 24029/len = 294/STRA = rev | | | | | | | |
| 3 | 104421_at | Cluster Incl U87147 Mus musculus flavin-containing monooxygenase 3 mRNA, complete cds/cds = (37,1641)/gb = U87147/ | 107.2 | A | 164.2 | P | 95.8 | A | 160.3 |
| | | gi = 1841861/ug = Mm 2900/len = 2020/STRA = for | | | | | | | |
| 3 | 92977_s_at | Cluster Incl X75330 H6 homeo box 3/cds = (307,1683)/gb = X75330/gi = 406586/ug = Mm 5016/len = 1972/STRA = for | 126 | P | 186.4 | P | 161.4 | A | 126.1 |
| 3 | 93710_at | Cluster Incl M37759: Mouse serine 1 ultra high sulfur protein gene, complete cds/cds = (0,692)/gb = M37759/gi = 200961/ | 500.9 | P | 499.1 | A | 518.7 | M | 704.2 |
| | | ug = Mm 86481/len = 693/STRA = for | | | | | | | |
| 3 | 96533_at | Cluster Incl AI508931 vc48e10 y1 Mus musculus cDNA, 5 end/clone = IMAGE-777834/clone_end = 5/gb = AI508931/ | 16.7 | A | 60.2 | A | 70.2 | A | 85.9 |
| | | gi = 4407836/ug = Mm 27464/len = 318/STRA = for | | | | | | | |
| 3 | 96583_at | Cluster Incl AI414051 ma05b11.x1 Mus musculus cDNA, 3 end/clone = IMAGE-303645/clone_end = 3/gb = AI414051/ | −2.6 | M | −21.9 | A | 82.8 | A | 32.2 |
| | | gi = 4257555/ug = Mm 29393/len = 473/STRA = rev | | | | | | | |
| 3 | 101333_at | Cluster Incl U73819 Mus musculus polypeptide GalNAc transferase-T4 (ppGaNTase-T4) mRNA, complete cds/ | −22.2 | A | −33.9 | A | 7.5 | A | −4.2 |
| | | cds = (0,1736)/gb = U73819/gi = 2121219/ug = Mm 56991/len = 1737/STRA = for | | | | | | | |
| 3 | 96968_at | Cluster Incl AB024521 Mus musculus mRNA for limitin, complete cds/cds = (160,708)/gb = AB024521/gi = 5821277/ | 369.1 | P | 406 | M | 433.6 | A | 345 |
| | | ug = Mm 88804/len = 997/STRA = for | | | | | | | |
| 4 | AFFX-BioC- | E Coli BioC 3 prime | 1257.3 | P | 1379.5 | P | 1133.7 | P | 1143.6 |
| | 3_at | | | | | | | | |
| 4 | 92547_at | Cluster Incl AB011081 Mus musculus mRNA for huntingtin interacting protein-2, complete cds/cds = (161,763)/ | 654.8 | P | 529.5 | P | 501.2 | P | 516.1 |
| | | gb = AB011081/gi = 2897817/ug = Mm.10082/len = 955/STRA = for | | | | | | | |
| 4 | 92622_at | Cluster Incl AI851064 UI-M-BH0-ajv-h-03-0-UI a1 Mus musculus cDNA, 3 end/clone = UI-M-BH0-ajv-h-03-0-UI/ | 497.4 | P | 369.6 | P | 405 | P | 475 |
| | | clone_end = 3/gb = AI851064/gi = 5494970/ug = Mm 11112/len = 316/STRA = for | | | | | | | |
| 4 | 92782_at | Cluster Incl U39074 Thymopoietin/cds = (237,1595)/gb = U39074/gi = 1335840/ug = Mm.124/len = 2170/STRA = for | 266 | P | 294 | P | 161.4 | P | 275.3 |
| 4 | 92808_f_at | Cluster Incl X17069 Mouse mRNA for protein with homology to transition protein 2 (TP2)/cds = (123,1358)/gb = X17069/ | 1332.1 | P | 1135.9 | P | 809.9 | P | 1003.3 |
| | | gi = 54875/ug = Mm 12758/len = 2026/STRA = for | | | | | | | |
| 4 | 92850_at | Cluster Incl AI836446 UI-M-AI0-aak-d-12-0-UI s1 Mus musculus cDNA, 3 end/clone = UI-M-AI0-aak-d-12-0-UI/ | 826.2 | P | 1147.6 | P | 887.1 | P | 922 |
| | | clone_end = 3/gb = AI836446/gi = 5470659/ug = Mm 13705/len = 451/STRA = for | | | | | | | |
| 4 | 93014_at | Cluster Incl Y17223 Mus musculus mRNA for F1F0-ATP synthase, g subunit/cds = (11,322)/gb = Y17223/gi = 3123645/ | 3881.8 | P | 4051.3 | P | 3228.2 | P | 3922.9 |
| | | ug = Mm 14663/len = 420/STRA = for | | | | | | | |
| 4 | 93028_at | Cluster Incl X58196 H19 fetal liver mRNA/cds = UNKNOWN/gb = X58196/gi = 51131/ug = Mm 14802/len = 1899/STRA = for | 4292.2 | P | 2538.8 | P | 2470.8 | P | 3217.5 |
| 4 | 93064_at | Cluster Incl AF035207 Mus musculus Nip21 mRNA, complete cds/cds = (133,1113)/gb = AF035207/gi = 2911347/ | 225.2 | P | 130.7 | P | 21 | P | 160.8 |
| | | ug = Mm.1561/len = 2213/STRA = for | | | | | | | |
| 4 | 93293_at | Cluster Incl M27844 Calmodulin/cds = (0,449)/gb = M27844/gi = 192364/ug = Mm 18041/len = 450/STRA = for | 4793.6 | P | 4969.5 | P | 4386.3 | P | 4590.5 |
| 4 | 93306_at | Cluster Incl U51196: Mus musculus APC-binding protein EB1 homolog mRNA, complete cds/cds = (66,872)/gb = U51196/ | 1247.4 | P | 1201.9 | P | 1190.3 | P | 1228.9 |
| | | gi = 1256433/ug = Mm.18418/len = 1203/STRA = for | | | | | | | |
| 4 | 93354_at | Cluster Incl Z22661 Apolipoprotein Cl/cds = (49,315)/gb = Z22661/gi = 410495/ug = Mm 189/len = 408/STRA = for | 1391.2 | P | 1108.4 | P | 1209.9 | P | 946.3 |
| 4 | 93365_s_at | Cluster Incl AI838149 UI-M-AL.0-abw-e-03-0-UI s1 Mus musculus cDNA, 3 end/clone = UI-M-AL0-abw-e-03-0-UI/ | 684.1 | P | 520.3 | P | 496.2 | P | 513.5 |
| | | clone_end = 3/gb = AI838149/gi = 5472362/ug = Mm 18972/len = 402/STRA = for | | | | | | | |
| 4 | 93366_r_at | Cluster Incl AI838149 UI-M-AL.0-abw-e-03-0-UI s1 Mus musculus cDNA, 3 end/clone = UI-M-AL0-abw-e-03-0-UI/ | 794.7 | P | 628.1 | P | 616.3 | P | 703 |
| | | clone_end = 3/gb = AI838149/gi = 5472362/ug = Mm 18972/len = 402/STRA = for | | | | | | | |
| 4 | 93579_at | Cluster Incl AW121613 UI-M-BH2 2-aoq-d-12-0-UI s1 Mus musculus cDNA, 3 end/clone = UI-M-BH2 2-aoq-d-12-0-UI/ | 693.7 | P | 682.9 | P | 544.5 | P | 655.8 |
| | | clone_end = 3/gb = AW121613/gi = 6096946/ug = Mm 2053/len = 135/STRA = for | | | | | | | |
| 4 | 93800_f_at | Cluster Incl AB013607 Mus musculus mRNA for c29, complete cds/cds = (21,1367)/gb = AB013607/gi = 3868801/ | 4552.6 | P | 3543.7 | P | 2209 | P | 3412.5 |
| | | ug = Mm 21408/len = 1539/STRA = for | | | | | | | |
| 4 | 93810_at | Cluster Incl X68378 Cathepsin D/cds = (0,1232)/gb = X68378/gi = 50302/ug = Mm 2147/len = 1233/STRA = for | 2220.4 | P | 2057.6 | P | 2064.2 | P | 2223.9 |
| 4 | 93822_at | Cluster Incl X73331 M musculus mRNA for ribosomal protein L37a/cds = (18,296)/gb = X73331/gi = 312413/ug = Mm 21529/ | 2128.5 | P | 1154 | P | 1148.5 | P | 1358.4 |
| | | len = 349/STRA = for | | | | | | | |
| 4 | 93844_at | Cluster Incl AW061302 UI-M-BH1-anw-c-09-0-UI s1 Mus musculus cDNA, 3 end/clone = UI-M-BH1-anw-c-09-0-UI/ | 3345.2 | P | 3961.5 | P | 3150.2 | P | 3983.2 |
| | | clone_end = 3/gb = AW061302/gi = 6009053/ug = Mm 21625/len = 321/STRA = for | | | | | | | |
| 4 | 93980_at | Cluster Incl AW124520 UI-M-BH2, 1-apo-f-11-0-UI.s1 Mus musculus cDNA, 3 end/clone = UI-M-BH2 1-apo-f-11-0-UI/ | 165.5 | P | 159.1 | P | 110.5 | P | 154.4 |
| | | clone_end = 3/gb = AW124520/gi = 6100050/ug = Mm 21693/len = 511/STRA = for | | | | | | | |
| 4 | 93987_f_at | Cluster Incl AW121568: UI-M-BH2 2-aop-g-06-0-UI s1 Mus musculus cDNA, 3 end/clone = UI-M-BH2 2-aop-g-06-0-UI/ | 5002.8 | P | 4605.9 | P | 4560.7 | P | 4654.9 |
| | | clone_end = 3/gb = AW121568/gi = 6096901/ug = Mm 21724/len = 329/STRA = for | | | | | | | |
| 4 | 94034_at | Cluster Incl AI839882: UI-M-AH0-acw-b-11-0-UI s1 Mus musculus cDNA, 3 end/clone = UI-M-AH0-acw-b-11-0-UI/ | 504.7 | P | 369.6 | P | 282 | P | 467.2 |
| | | clone_end = 3/gb = AI839882/gi = 5474095/ug = Mm 21911/len = 353/STRA = for | | | | | | | |
| 4 | 94042_f_at | Cluster Incl AI843937: UI-M-AP1-agh-b-10-0-UI a1 Mus musculus cDNA, 3 end/clone = UI-M-AP1-agh-b-10-0-UI/ | 4580.2 | P | 4484.7 | P | 4409.6 | P | 4274.6 |
| | | clone_end = 3/gb = AI843937/gi = 5478150/ug = Mm 21955/len = 356/STRA = for | | | | | | | |
| 4 | 94056_at | Cluster Incl M21285 Stearoyl-coenzyme A desaturase 1/cds = (0,1067)/gb = M21285/gi = 200949/ug = Mm 22026/len = 1068/ | 3235.1 | P | 3395.8 | P | 4014.2 | P | 3236 |
| | | STRA = for | | | | | | | |
| 4 | 94057_g_at | Cluster Incl M21285: Stearoyl-coenzyme A desaturase 1/cds = (0,1067)/gb = M21285/gi = 200949/ug = Mm.22026/len = 1068/ | 2000.3 | P | 2079.8 | P | 2980.8 | P | 1800.1 |
| | | STRA = for | | | | | | | |
| 4 | 94218_at | Cluster Incl M20130: Mouse t complex polypeptide 1 (Tcp-1-a) mRNA, 3 end/cds = (0,714)/gb = M20130/gi = 201722/ | 357 | P | 302.6 | P | 289.9 | P | 362.3 |
| | | ug = Mm 2223/len = 863/STRA = for | | | | | | | |
| 4 | 94276_at | Cluster Incl AF064635: Mus musculus putative ateroid dehydrogenase (KIK-I) mRNA, complete cds/cds = (19,957)/ | 966 | P | 903.9 | P | 713.7 | P | 943.7 |
| | | gb = AF064635/gi = 3142701/ug = Mm.22505/len = 978/STRA = for | | | | | | | |
| 4 | 94312_at | Cluster Incl AI180784: ub76c07.r1 Mus musculus cDNA, 5 and/clone = IMAGE-1383660/clone_end = 5/gb = AI180784/ | 685.7 | P | 738.8 | P | 558.9 | P | 542.3 |
| | | gi = 3731422/ug = Mm 22635/len = 360/STRA = rev | | | | | | | |
| 4 | 94819_f_at | Cluster Incl AF005886: Mus musculus cyclin I mRNA, complete cds/cds = (286,1419)/gb = AF005888/gi = 4101686/ | 1773.5 | P | 1762.5 | P | 1776.3 | P | 1550.3 |
| | | ug = Mm 22711/len = 1548/STRA = for | | | | | | | |
| 4 | 95437_at | Cluster Incl AI836139 UI-M-AQ0-aai-b-06-0-UI.a2 Mus musculus cDNA, 3 end/clone = UI-M-AQ0-aai-b-06-0-UI/ | 573 | P | 497.3 | P | 463.8 | P | 496.5 |
| | | clone_end = 3/gb = AI836139/gi = 5470352/ug = Mm 24538/len = 395/STRA = for | | | | | | | |
| 4 | 95442_at | Cluster Incl AI835706: UI-M-AIO-aan-b-08-0-UI s1 Mus musculus cDNA, 3 end/clone = UI-M-AI0-aan-b-08-0-UI/ | 589.6 | P | 484.6 | P | 309.2 | P | 432.3 |
| | | clone_end = 3/gb = AI835706/gi = 5469919/ug = Mm 24571/len = 329/STRA = for | | | | | | | |
| 4 | 95457_at | Cluster Incl AI854214 UI-M-BH0-ajz-e-09-0-UI s1 Mus musculus cDNA, 3 end/clone = UI-M-BH0-ajz-e-09-0-UI/ | 676.3 | P | 713.8 | P | 583 | P | 671.9 |
| | | clone_end = 3/gb = AI854214/gi = 5498120/ug = Mm 24703/len = 647/STRA = for | | | | | | | |
| 4 | 95470_at | Cluster Incl AI846025: UI-M-AP1-agi-g-04-0-UI s1 Mus musculus cDNA, 3 end/clone = UI-M-AP1-agi-g-04-0-UI/ | 477.4 | P | 293.4 | P | 281.1 | P | 383.5 |
| | | clone_end = 3/gb = AI846025/gi = 5489931/ug = Mm 24789/len = 451/STRA = for | | | | | | | |
| 4 | 96027_at | Cluster Incl AW120546: UI-M-BH1-ana-e-02-0-UI.s2 Mus musculus cDNA, 3 end/clone = UI-M-BH1-ana-e-02-0-UI/ | 246.5 | P | 254 | P | 209.3 | P | 226.9 |
| | | clone_end = 3/gb = AW120546/gi = 6095879/ug = Mm 25732/len = 473/STRA = for | | | | | | | |
| 4 | 96050_at | Cluster Incl AJ011740 Mus musculus mRNA for integrase interacter 1a protein (INI1A)/cds = (193,1350)/gb = AJ011740/ | 597.6 | P | 562.6 | P | 576.4 | P | 522 |
| | | gi = 4138222/ug = Mm 26116/len = 1622/STRA = for | | | | | | | |
| 4 | 96068_at | Cluster Incl AI848821: UI-M-AJ1-ahb-a-11-0-UI s1 Mus musculus cDNA, 3 end/clone = UI-M-AJ1-ahb-a-11-0-UI/ | 311 | P | 226.2 | P | 232.7 | P | 259.9 |
| | | clone_end = 3/gb = AI848821/gi = 5492727/ug = Mm 26418/len = 401/STRA = for | | | | | | | |
| 4 | 96653_at | Cluster Incl AI851762: UI-M-BH0-aim-d-03-0-UI.s1 Mus musculus cDNA, 3 end/clone = UI-M-BH0-aim-d-03-0-UI/ | 3893 | P | 2639 | P | 1920.1 | P | 1941.4 |
| | | clone_end = 3/gb = AI851762/gi = 5495668/ug = Mm 27336/len = 419/STRA = for | | | | | | | |
| 4 | 96674_at | Cluster Incl AW123553: UI-M-BH2 1-aqb-f-12-0-UI s1 Mus musculus cDNA, 3 end/clone = UI-M-BH2 1-aqb-f-12-0-UI/ | 921.8 | P | 897.1 | P | 659 | P | 899.9 |
| | | clone_end = 3/gb = AW123553/gi = 6099083/ug = Mm 27404/len = 400/STRA = for | | | | | | | |
| 4 | 96677_at | Cluster Incl AI849566: UI-M-AH1-agr-c-04-0-UI s1 Mus musculus cDNA, 3 end/clone = UI-M-AH1-agr-c-04-0-UI/ | 807.9 | P | 629.3 | P | 547.7 | P | 714.5 |
| | | clone_end = 3/gb = AI849566/gi = 5493472/ug = Mm 27424/len = 351/STRA = for | | | | | | | |
| 4 | 96684_at | Cluster Incl AI846123: UI-M-AN1-afj-a-11-0-UI s2 Mus musculus cDNA, 3 end/clone = UI-M-AN1-afj-a-11-0-UI/ | 838.4 | P | 231.9 | P | 349.9 | P | 711 |
| | | clone_end = 3/gb = AI846123/gi = 5490029/ug = Mm 27453/len = 341/STRA = for | | | | | | | |
| 4 | 97206_at | Cluster Incl AW230369: uo62g07 y1 Mus musculus cDNA, 5 end/clone = IMAGE-2647164/clone_end = 5/gb = AW230369/ | 711.4 | P | 470.2 | P | 471.7 | P | 544.8 |
| | | gi = 6559685/ug = Mm 27700/len = 692/STRA = for | | | | | | | |
| 4 | 98894_at | Cluster Incl AA867655 vx12e03 r1 Mus musculus cDNA, 5 end/clone = IMAGE-1264252/clone_end = 5/gb = AA867655/ | 782.5 | P | 615.8 | P | 504.1 | P | 611.3 |
| | | gi = 2963100/ug = Mm 28550/len = 428/STRA = for | | | | | | | |
| 4 | 98901_at | Cluster Incl AW047870: UI-M-BH1-afs-f-10-0-UI s1 Mus musculus cDNA, 3 end/clone = UI-M-BH1-afs-f-10-0-UI/ | 556.9 | P | 556.3 | P | 562.3 | P | 592.3 |
| | | clone_end = 3/gb = AW047870/gi = 5908399/ug = Mm 28578/len = 353/STRA = for | | | | | | | |
| 4 | 94456_at | Cluster Incl AI841497: UI-M-AH0-acy-d-12-0-UI s1 Mus musculus cDNA, 3 end/clone = UI-M-AH0-acy-d-12-0-UI/ | 1760.2 | P | 1792.6 | P | 1284.8 | P | 1667.6 |
| | | clone_end = 3/gb = AI841497/gi = 5475710/ug = Mm 28805/len = 411/STRA = for | | | | | | | |
| 4 | 94466_f_at | Cluster Incl U19891 CCAAT/enhancer binding protein alpha (C/EBP), related sequence 1/cds = (11,3004)/gb = U19891/ | 470.1 | P | 407.5 | P | 294.2 | P | 362.9 |
| | | gi = 790574/ug = Mm 28845/len = 3302/STRA = for | | | | | | | |
| 4 | 94528_at | Cluster Incl AI846206 UI-M-AM1-agb-e-11-0-UI s1 Mus musculus cDNA, 3 end/clone = UI-M-AM1-agb-e-11-0-UI/ | 336.7 | P | 319.4 | P | 236.9 | P | 294.8 |
| | | clone_end = 3/gb = AI846206/gi = 5490112/ug = Mm 29037/len = 141/STRA = for | | | | | | | |
| 4 | 94548_at | Cluster Incl AI790537 ul02d10 x1 Mus musculus cDNA, 3 end/clone = IMAGE-2064883/clone_end = 3/gb = AI790537/ | 813 | P | 459.5 | P | 744.6 | P | 453.7 |
| | | gi = 5338253/ug = Mm.29078/len = 440/STRA = for | | | | | | | |
| 4 | 95058_f_at | Cluster Incl AW121984 UI-M-BH2 3-aoj-b-10-0-UI s1 Mus musculus cDNA, 3 end/clone = UI-M-BH2 3-aoj-b-10-0-UI/ | 1908.8 | P | 2078 | P | 1684.8 | P | 1919.6 |
| | | clone_end = 3/gb = AW121984/gi = 6097345/ug = Mm.29153/len = 390/STRA = for | | | | | | | |
| 4 | 95118_r_at | Cluster Incl AI131895 ue97e08 x1 Mus musculus cDNA, 3 end/clone = IMAGE-1499078/clone_end = 3/gb = AI131895/ | 768 | P | 854.2 | P | 597.6 | P | 761.2 |
| | | gi = 3601911/ug = Mm.29381/len = 397/STRA = for | | | | | | | |
| 4 | 95686_at | Cluster Incl AW047017 UI-M-BH1-alp-h-05-0-UI s1 Mus musculus cDNA, 3 end/clone = UI-M-BH1-alp-h-05-0-UI/ | 237.2 | P | 206.5 | P | 202.1 | P | 242.3 |
| | | clone_end = 3/gb = AW047017/gi = 5907546/ug = Mm.29641/len = 353/STRA = for | | | | | | | |
| 4 | 95702_at | Cluster Incl AI461803 uc43c04.x1 Mus musculus cDNA, 3 end/clone = IMAGE-1400742/clone_end = 3/gb = AI461803/ | 1003.2 | P | 904.2 | P | 721.7 | P | 832.7 |
| | | gi = 4315833/ug = Mm.29697/len = 481/STRA = for | | | | | | | |
| 4 | 95744_at | Cluster Incl U13837: Mus musculus vacuolar adenosine triphosphatase subunit A gene, complete cds/cds = (0,1853)/ | 580.5 | P | 356.8 | P | 691.8 | P | 607.6 |
| | | gb = U13837/gi = 1184658/ug = Mm.29771/len = 1854/STRA = for | | | | | | | |
| 4 | 95745_g_at | Cluster Incl U13837 Mus musculus vacuolar adenosine triphosphatase subunit A gene, complete cds/cds = (0,1853)/ | 738.4 | P | 668.7 | P | 472.8 | P | 749.2 |
| | | gb = U13837/gi = 1184658/ug = Mm.29771/len = 1854/STRA = for | | | | | | | |
| 4 | 95749_at | Cluster Incl AW122364 UI-M-BH2.2-aow-a-11-0-UI.s1 Mus musculus CDNA, 3 end/clone = UI-M-BH2.2-aow-a-11-0-UI/ | 417 | P | 287.9 | P | 445.7 | P | 395.4 |
| | | clone_end = 3/gb = AW122364/gi = 6097859/ug = Mm 29778/len = 488/STRA = for | | | | | | | |
| 4 | 95758_at | Cluster Incl M26270 Stearoyl-coenzyme A desaturase 2/cds = (300,1376)/gb = M26270/gi = 200933/ug = Mm.298/len = 5032/ | 2940.7 | P | 3044.3 | P | 2038.9 | P | 1898.5 |
| | | STRA = for | | | | | | | |
| 4 | 96270_at | Cluster Incl AI847092: UI-M-AP1-agn-e-07-0-U1 s1 Mus musculus CDNA, 3 end/clone = UI-M-AP1-agn-e-07-0-UI/ | 1564.8 | P | 1566 | P | 1679.4 | P | 1565.6 |
| | | clone_end = 3/gb = AI847092/gi = 5490998/ug = Mm 29852/len = 261/STRA = for | | | | | | | |
| 4 | 96337_at | Cluster Incl AF033350: Mus musculus CDCREL-1 homolog (Cdcrel-1) MRNA, partial cds/cds = (0,1044)/gb = AF033350/ | 420.4 | P | 372.7 | P | 374.3 | P | 370 |
| | | gi = 3986408/ug = Mm 29976/len = 1973/STRA = for | | | | | | | |
| 4 | 96343_at | Cluster Incl AI836968 UI-M-AJ0-aaz-c-09-0-UI s1 Mus musculus CDNA, 3 end/clone = UI-M-AJ0-aaz-c-09-0-UI/ | 1859.1 | P | 1810 | P | 1898 | P | 1782.4 |
| | | clone_end = 3/gb = AI836968/gi = 5471181/ug = Mm.29991/len = 445/STRA = for | | | | | | | |
| 4 | 96849_at | Cluster Incl AA655369: vs24h12 r1 Mus musculus CDNA, 5 end/clone = IMAGE-1139207/clone_end = 5/gb = AA655369/ | 371.6 | P | 313.4 | P | 240.7 | P | 346.4 |
| | | gi = 2591523/ug = Mm 30034/len = 545/STRA = for | | | | | | | |
| 4 | 96943_at | Cluster Incl AW125234 UI-M-BH2 1-apa-e-0300-UI s1 Mus musculus cDNA, 3 end/clone = UI-M-BH2 1-apa-e-03-0-UI/ | 1027.9 | P | 1008.9 | P | 689.6 | P | 977.8 |
| | | clone_end = 3/gb = AW125234/gi = 6100764/ug = Mm 30195/len = 310/STRA = for | | | | | | | |
| 4 | 97456_at | Cluster Incl AI838021: UI-M-AP0-abl-g-11-0-UI.s1 Mus musculus cDNA, 3 end/clone = UI-M-AP0-abl-g-11-0-UI/ | 816.3 | P | 615.8 | P | 589.5 | P | 588 |
| | | clone_end = 3/gb = AI838021/gi = 5472234/ug = Mm.30263/len = 392/STRA = for | | | | | | | |
| 4 | 97459_at | Cluster Incl AI851441: UI-M-BH0-akj-e-05-0-UI.s1 Mus musculus cDNA, 3 end/clone = UI-M-BH0-akj-e-05-0-UI/ | 1591.7 | P | 1255.2 | P | 1116.1 | P | 1446.3 |
| | | clone_end = 3/gb = AI851441/gi = 5495347/ug = Mm.30270/len = 338/STRA = for | | | | | | | |
| 4 | 97497_at | Cluster Incl Z11886 Notch gene homolog 1, (Drosphila)/cds = (78,7673)/gb = Z11886/gi = 288502/ug = Mm.31255/len = 8064/ | 812 | P | 682.6 | P | 506 | P | 770.3 |
| | | STRA = for | | | | | | | |
| 4 | 97504_at | Cluster Incl M83749 Cyclin D2/cds = (270,1139)/gb = M83749/gi = 192938/ug = Mm 3141/len = 1255/STRA = for | 1703.2 | P | 1415.1 | P | 855.3 | P | 1026.4 |
| 4 | 97512_at | Cluster Incl AW226650 um58g10.y1 Mus musculus cDNA, 5 end/clone = IMAGE-2270274/clone_end = 5/gb = AW226650/ | 2504.9 | P | 2252.5 | P | 2073.1 | P | 2306.7 |
| | | gi = 6555946/ug = Mm 318/len = 390/STRA = for | | | | | | | |
| 4 | 98053_at | Cluster Incl AF058797 Mus musculus 14-3-3 protein beta mRNA, complete cds/cds = (171,911)/gb = AF058797/gi = 3065924/ | 1589.4 | P | 1678.4 | P | 1456.2 | P | 1576.6 |
| | | ug = Mm 34319/len = 1022/STRA = for | | | | | | | |
| 4 | 98056_at | Cluster Incl AI846214 UI-M-AM1-agb-f-10-0-UI s1 Mus musculus cDNA, 3 end/clone = UI-M-AM1-agb-f-10-0-UI/ | 982 | P | 1080.2 | P | 728.2 | P | 1043 |
| | | clone _end = 3/gb = AI846214/gi = 5490120/ug = Mm 34346/len = 343/STRA = for | | | | | | | |
| 4 | 98077_at | Cluster Incl AA796831: vp15g02 r1 Mus musculus cDNA, 5 end/clone = IMAGE-1068722/clone_end = 5/gb = AA796831/ | 378.9 | P | 161 | P | 110.2 | P | 240.6 |
| | | gi = 2859786/ug = Mm 34549/len = 424/STRA = for | | | | | | | |
| 4 | 98111_at | Cluster Incl L40406 Heat shock protein, 105 kDa/cds = (139,2718)/gb = L40406/gi = 840651/ug = Mm 34828/len = 3387/ | 772.9 | P | 378.4 | P | 242.6 | P | 342.4 |
| | | STRA = for | | | | | | | |
| 4 | 98112_r_at | Cluster Incl AI839225 UI-M-AK0-adh-d-01-0-UI s1 Mus musculus cDNA, 3 end/clone = UI-M-AK0-adh-d-01-0-UI/ | 482.9 | P | 381.5 | P | 283.2 | P | 352.4 |
| | | clone_end = 3/gb = AI839225/gi = 5473438/ug = Mm 34832/len = 429/STRA = for | | | | | | | |
| 4 | 98113_at | Cluster Incl U60824: Mus musculus proteasome beta-subunit C5 (Pamb1) mRNA, allele B, complete cds/cds = (29,751)/ | 2596.2 | P | 2543 | P | 2180.4 | P | 2199.9 |
| | | gb = U60824/gi = 1698583/ug = Mm 34837/len = 812/STRA = for | | | | | | | |
| 4 | 98122_at | Cluster Incl AF074600 Mus musculus LIM domain transcription factor LMO4 mRNA, complete cds/cds = (0,497)/ | 1961.1 | P | 1868.4 | P | 1885.8 | P | 1847.6 |
| | | gb = AF074600/gi = 3702853/ug = Mm.35004/len = 498/STRA = for | | | | | | | |
| 4 | 98532_at | Cluster Incl AF011644 Mus musculus oral tumuor suppressor homolog (Doc-1) mRNA, partial cds/cds = (0,330)/ | 1621.9 | P | 1464.3 | P | 1357.9 | P | 1553.9 |
| | | gb = AF011644/gi = 2305225/ug = Mm 35847/len = 981/STRA = for | | | | | | | |
| 4 | 98555_at | Cluster Incl AB008516: Tetratricopeptide repeat domain/cds = (11,5950)/gb = AB008516/gi = 3308963/ug = Mm 3679/ | 603.2 | P | 358 | P | 369.6 | P | 322.8 |
| | | len = 7172/STRA = for | | | | | | | |
| 4 | 98602_at | Cluster Incl U08110 RAN GTPase activating protein 1/cds = (274,2043)/gb = U08110/gi = 472851/ug = Mm 3833/len = 2981/ | 604 | P | 407.5 | P | 373.3 | P | 430.7 |
| | | STRA = for | | | | | | | |
| 4 | 99098_at | Cluster Incl AW045533 UI-M-BH1-aks-c-08-0-UI s1 Mus musculus cDNA, 3 end/clone = UI-M-BH1-aks-c-08-0-UI/ | 1387.2 | P | 1562.6 | P | 614.7 | P | 1017.1 |
| | | clone_end = 3/gb = AW045533/gi = 5906062/ug = Mm 39472/len = 426/STRA = for | | | | | | | |
| 4 | 99128_at | Cluster Incl AI849767 UI-M-AL1-ahn-f-09-0-UI s1 Mus musculus cDNA, 3 end/clone = UI-M-AL1-ahn-f-09-0-UI/ | 2818.3 | P | 2815.2 | P | 2455.3 | P | 2566 |
| | | clone_end = 3/gb = AI849767/gi = 5493673/ug = Mm 41/len = 362/STRA = for | | | | | | | |
| 4 | 99147_at | Cluster Incl AI843686 UI-M-AP1-agh-05-0-UI s1 Mus musculus cDNA, 3 end/clone = UI-M-AP1-agh-g-05-0-UI/ | 1413.7 | P | 1136.7 | P | 1058.4 | P | 1171.4 |
| | | clone_end = 3/gb = AI843986/gi = 5478199/ug = Mm 41486/len = 246/STRA = for | | | | | | | |
| 4 | 99569_at | Cluster Incl AF021836 Mus muscules hair keratin basic 5 (Hb5) mRNA, complete cds/cds = (61,1584)/gb = AF021836/ | 2674.5 | P | 1423.8 | P | 1008.4 | P | 1766.7 |
| | | gi = 4103155/ug = Mm 42251/len = 2344/STRA = for | | | | | | | |
| 4 | 99583_at | Cluster Incl X53451 Glutathione S-transferase, pi 2/cds = (8,640)/gb = X53451/gi = 51124/ug = Mm 426/len = 703/STRA = for | 1567.2 | P | 1532.6 | P | 1345.9 | P | 1281.2 |
| 4 | 99607_at | Cluster Incl Z47088: Transcription elongation factor B (SIII), polypeptide 1 (15 KDa),-like/cds = UNKNOWN/gb = Z47088/ | 1621.2 | P | 1349.6 | P | 1069.7 | P | 1276.3 |
| | | gi = 861057/ug = Mm.42944/len = 1219/STRA = for | | | | | | | |
| 4 | 99631_f_at | Cluster Incl U08440 Mus musculus Balb/c cytochrome c oxidase subunit VIaL mRNA, complete cds/cds = (72,332)/ | 3996.2 | P | 3974.8 | P | 3676.9 | P | 3506.8 |
| | | gb = U08440/gi = 475706/ug = Mm.43415/len = 521/STRA = for | | | | | | | |
| 4 | 99672_at | Cluster Incl Z49916: M.muscules mRNA for CLCN4/cds = (377,2620)/gb = Z49916/gi = 929679/ug = Mm 4408/len = 2739/ | 303 | P | 286.1 | P | 216.7 | P | 303.1 |
| | | STRA = for | | | | | | | |
| 4 | 99674_at | Cluster Incl AW060297 UI-M-BH1-amv-c-04-0-UI s1 Mus musculus cDNA, 3 end/clone = UI-M-BH1-amv-c-04-0-UI/ | 694.2 | P | 460.2 | P | 674.3 | P | 663.8 |
| | | clone_end = 3/gb = AW060297/gi = 6008013/ug = Mm 44174/len = 415/STRA = for | | | | | | | |
| 4 | 100066_at | Cluster Incl U20892 Phoshonbosylglycinamide formyltransferase/cds = (115,3147)/gb = U20892/gi = 2182233/ug = Mm 4505/ | 556.8 | P | 607.5 | P | 397.4 | P | 562 |
| | | len = 3296/STRA = for | | | | | | | |
| 4 | 100091_at | Cluster Incl D87990 House mouse, Musculus domesticus mRNA for UDP-galactose transporter related isozyme 1, | 1350.6 | P | 1246 | P | 829.3 | P | 1363 |
| | | complete cds/cds = (88,1056)/gb = D87990)/gi = 1694638/ug = Mm.4593/len = 1169/STRA = for | | | | | | | |
| 4 | 100550_f_at | Cluster Incl AW060422: UI-M-BH1-anj-c-07-0-UI s1 Mus musculus cDNA, 3 end/clone = UI-M-BH1-anj-c-07-0-UI/ | 4110.6 | P | 4032.2 | P | 3877.9 | P | 3634.3 |
| | | clone_end = 3/gb = AW060422/gi = 6008173/ug = Mm 548/len = 373/STRA = for | | | | | | | |
| 4 | 100567_at | Cluster Incl M20497 Mouse adipose fatty acid binding protein (422) gene, 5 end/cds = (65,97)/gb = M20497/gi = 191743/ | 2400.5 | P | 2219.7 | P | 2069.4 | P | 2285.5 |
| | | ug = Mm 582/len = 98/STRA = for | | | | | | | |
| 4 | 101044_at | Cluster Incl X13752 Delta-aminolevulinate dehydratase/cds = (6,998)/gb = X13752/gi = 52951/ug = Mm 6988/len = 1080/ | 749.9 | P | 605.1 | P | 720.7 | P | 448.3 |
| | | STRA = for | | | | | | | |
| 4 | 101051_at | Cluster Incl AF004941 Mus musculus S100A3 calcuim binding protein mRNA, complete cds/cds = (59,364)/gb = AF004941/ | 4124.5 | P | 2320.9 | P | 1214.2 | P | 2472.5 |
| | | gi = 3916049/ug = Mm 703/len = 712/STRA = for | | | | | | | |
| 4 | 101052_g_at | Cluster Incl AF004941 Mus musculus S100A3 calcuim binding protein mRNA, complete ccds/cds = (59,364)/gb = AF004941/ | 3096.1 | P | 1105.9 | P | 332.7 | P | 1275.1 |
| | | gi = 3916049/ug = Mm 703/len = 712/STRA = for | | | | | | | |
| 4 | 101530_at | Cluster Incl U97079 Mus musculus US-116 KD mRNA, complete cds/cds = (27,2942)/gb = U97079/gi = 2276461/ug = Mm 873/ | 458.1 | P | 535.4 | P | 495 | P | 670.8 |
| | | len = 3271/STRA = for | | | | | | | |
| 4 | 101561_at | Cluster Incl K02236 Metallothionein 2/cds = (0,185)/gb = K02236/gi = 199131/ug = Mm.89170/len = 186/STRA = for | 4972 | P | 3765.8 | P | 4080.3 | P | 3711.9 |
| 4 | 101569_at | Cluster Incl AB033744 Mus musculus mRNA for type II cytokeratin, complete cds/cds = (51,1625)/gb = AB0033744/ | 4668.3 | P | 3796.1 | P | 1711.3 | P | 3670 |
| | | gi = 6092074/ug = Mm 89769/len = 2193/STRA = for | | | | | | | |
| 4 | 101581_at | Cluster Incl U82122.Ubiqutin conjugating enzyme E3A/cds = (0,2549)/gb = U82122/gi = 1843534/ug = Mm 9002/len = 2550/ | 320.2 | P | 366.8 | P | 205.3 | P | 275.3 |
| | | STRA = for | | | | | | | |
| 4 | 101984_at | Cluster Incl AF004591 Mus musculus copper transport protein Atox1 (ATOX1) mRNA, complete cds/cds = (38,244)/ | 3043.9 | P | 3149.6 | P | 2704.4 | P | 2819.5 |
| | | gb = AF004591/gi = 2209342/ug = Mm.9703/len = 432/STRA = for | | | | | | | |
| 4 | 102009_at | Cluster Incl AI835274 UI-M-AQ0-aab-c-12-0-UI s1 Mus musculus cDNA, 3 end/clone = UI-M-AQ0-aab-c-12-0-UI/ | 282.8 | P | 264.8 | P | 254.7 | P | 185.7 |
| | | clone_end = 3/gb = AI835274/gi = 5469487/ug = Mm.100115/len = 460/STRA = rev | | | | | | | |
| 4 | 102056_f_at | Cluster Incl AA839379: vw48g12.r1 Mus musculus cDNA, 5 end/clone = IMAGE-1247110/clone_end = 5/gb = AA839379/ | 298.4 | P | 273.5 | P | 193.5 | P | 201.6 |
| | | gi = 2915474/ug = Mm.10374/len = 500/STRA = for | | | | | | | |
| 4 | 102062_at | Cluster Incl U85614: Mus musculus SRG3 mRNA, complete cds/cds = (60,3362)/gb = U85614/gi = 1816634/ug = Mm 1050/ | 314.3 | P | 237.6 | P | 114.7 | P | 337.3 |
| | | len = 4765/STRA = for | | | | | | | |
| 4 | 102069_at | Cluster Incl S78454 Mus musculus metal response element DNA-binding protein M96 mRNA, complete cds/cds = (0,1121)/ | 420.9 | P | 269.6 | P | 214.5 | P | 401.6 |
| | | gb = S78454/gi = 998845/ug = Mm.10563/len = 1517/STRA = for | | | | | | | |
| 4 | 102754_at | Cluster Incl Y17267: Mus musculus mRNA for ubiquitin conjugating enzyme/cds = (258,14795)/gb = Y17267/gi = 3319989/ | 260 | P | 187.6 | P | 184 | P | 186.8 |
| | | ug = Mm 12921/len = 15475/STRA = for | | | | | | | |
| 4 | 102793_at | Cluster Incl D86423 Mus musculus mRNA for HGT keratin, partial cds/cds = (0,163)/gb = D86423/gi = 1944398/ | 2031.6 | P | 398.7 | P | 111.5 | P | 598 |
| | | ug = Mm.13979/len = 508/STRA = for | | | | | | | |
| 4 | 103067_at | Cluster Incl Z48587: M musculus ral-A mRNA/cds = (0,620)/gb = Z48587/gi = 1107741/ug = Mm 16779/len = 621/STRA = for | 379.4 | P | 411.9 | P | 316.6 | P | 398.5 |
| 4 | 103341_at | Cluster Incl U49350.Mus musculus CTP synthetase mRNA, complete cds/cds = (111,1886)/gb = U49350/gi = 1515356/ | 1363.2 | P | 1018.6 | P | 685.5 | P | 884.7 |
| | | ug = Mm 1815/len = 2681/STRA = for | | | | | | | |
| 4 | 103344_at | Cluster Incl L16953: Mouse tumor cell dna-j-like protein 1 mRNA, complete cds/cds = (7,1665)/gb = L16953/gi = 473846/ | 358 | P | 279 | P | 296.7 | P | 278 |
| | | ug = Mm 18237/len = 1792/STRA = for | | | | | | | |
| 4 | 103413_at | Cluster Incl AI021091 ub01b04 r1 Mus musculus cDNA, 5 end/clone = IMAGE-1365679/clone_end = 5/gb = AI021091/ | 237.3 | P | 145.2 | P | 157.4 | P | 277.8 |
| | | gi = 3235427/ug = Mm 18834/len = 426/STRA = for | | | | | | | |
| 4 | 103415_at | Cluster Incl AI840925: UI-M-AM0-adm-g-07-0-UI.s1 Mus musculus cDNA, 3 end/clone = UI-M-AM0-adm-g-07-0-UI/ | 216.5 | P | 127.3 | P | 152.8 | P | 179.1 |
| | | clone_end = 3/gb = AI840925/gi = 5475138/ug = Mm 18847/len = 279/STRA = for | | | | | | | |
| 4 | 103619_at | Cluster Incl AI850017: UI-M-BG0-aib-g-03-0-UI.s1 Mus musculus cDNA, 3 end/clone = UI-M-BG0-aib-g-03-0-UI/ | 480.9 | P | 466.6 | P | 345.4 | P | 530.1 |
| | | clone_end = 3/gb = AI850017/gi = 5493923/ug = Mm 20242/len = 428/STRA = rev | | | | | | | |
| 4 | 104317_at | Cluster Incl AW123099: UI-M-BH2 1-apc-e-04-0-UI s1 Mus musculus cDNA, 3 end/clone = UI-M-BH2 1-apc-e-04-0-UI/ | 310.5 | P | 307 | P | 225.5 | P | 304.2 |
| | | clone_end = 3/gb = AW123099/gi = 6098629/ug = Mm 22417/len = 610/STRA = for | | | | | | | |
| 4 | 94418_at | Cluster Incl AI839004: UI-M-AN0-acm-c-04-0-UI.s1 Mus musculus cDNA, 3 end/clone = UI-M-AN0-acm-c-04-0-UI/ | 614.9 | P | 370.1 | P | 169.2 | P | 312.6 |
| | | clone_end = 3/gb = AI839004/gi = 5473217/ug = Mm 26171/len = 428/STRA = for | | | | | | | |
| 4 | 94970_at | Cluster Incl AI852314 UI-M-BH0-aje-g-07-0-UI.s1 Mus musculus cDNA, 3 end/clone = UI-M-BH0-aje-g-07-0-UI/ | 171.6 | P | 149.5 | P | 97.5 | P | 102.9 |
| | | clone_end = 3/gb = AI852314/gi = 5496220/ug = Mm 27223/len = 401/STRA = for | | | | | | | |
| 4 | 95002_at | Cluster Incl AI838731 UI-M-AO0-acf-c-07-0-UI.s1 Mus musculus cDNA, 3 end/clone = UI-M-AO0-acf-c-07-0-UI/ | 644.5 | P | 611.4 | P | 611 | P | 678.1 |
| | | clone_end = 3/gb = AI838731/gi = 5472944/ug = Mm 27362/len = 328/STRA = for | | | | | | | |
| 4 | 95526_at | Cluster Incl AI606243 vo02e01 x1 Mus musculus cDNA, 3 end/clone = IMAGE-1040280/clone_end = 3/gb = AI606243/ | 396.2 | P | 348.1 | P | 393.7 | P | 307.8 |
| | | gi = 4615410/ug = Mm 27607/len = 356/STRA = for | | | | | | | |
| 4 | 95619_at | Cluster Incl AI851723 UI-M-BH0-aio-f-07-0-UI s1 Mus musculus cDNA, 3 end/clone = UI-M-BH0-aio-f-07-0-UI/ | 175 | P | 193 | P | 109.5 | P | 127.9 |
| | | clone_end = 3/gb = AI851723/gi = 5495624/ug = Mm 28037/len = 391/STRA = for | | | | | | | |
| 4 | 96756_at | Cluster Incl AA693236: vr58d08 r1 Mus musculus cDNA, 5 end/clone = IMAGE-1124847/clone_end = 5/gb = AA693236/ | 200.3 | P | 136.4 | P | 119.7 | P | 166.2 |
| | | gi = 2694174/ug = Mm 28983/len = 409/STRA = for | | | | | | | |
| 4 | 97918_at | Cluster Incl AA623587 vn73d05 r1 Mus musculus cDNA, 5 end/clone = IMAGE-1037577/clone_end = 5/gb = AA623587/ | 574.4 | P | 502.3 | P | 504.7 | P | 445.6 |
| | | gi = 2527483/ug = Mm.30122/len = 163/STRA = for | | | | | | | |
| 4 | 97954_at | Cluster Incl X59513 Mouse 5 end of TRP1 gene for tyrosinase-related protein-1/cds = (181,183)/gb = X59513/gi = 54913/ | 332.9 | P | 94.2 | P | 35 | P | 148.2 |
| | | ug = Mm 30438/len = 272/STRA = for | | | | | | | |
| 4 | 98024_at | Cluster Incl X55316 Nuclear transcription factor-Y beta/cds = (209,832)/gb = X55316/gl = 53362/ug = Mm 3259/len = 1234/ | 480.5 | P | 245.5 | P | 206.1 | P | 253.8 |
| | | STRA = for | | | | | | | |
| 4 | 98404_at | Cluster Incl X64587 M. musculus mRNA for splicing factor U2AF (65 kD)/cds = UNKNOWN/gb = X64587/gi = 55100/ | 2111.8 | P | 1898.7 | P | 1936.4 | P | 1970.3 |
| | | ug = Mm 3358/len = 2119/STRA = for | | | | | | | |
| 4 | 98415_at | Cluster Incl AF046060 Mus musculus cytokine receptor related protein 4 (Cytor4) mRNA, complete cds/cds = (54,1382)/ | 360.1 | P | 487.2 | P | 441.1 | P | 410.4 |
| | | gb = AF046060/gi = 4105473/ug = Mm.33962/len = 2365/STRA = for | | | | | | | |
| 4 | 98997_f _at | Cluster Incl AF020790 Mus musculus hair keratin acidic 5 (Ha5) mRNA, complete cds/cds = (129,1406)/gb = AF020790/ | 2036.1 | P | 1114.4 | P | 850.1 | P | 1291.7 |
| | | gi = 4103157/ug = Mm.37952/len = 1737/STRA = rev | | | | | | | |
| 4 | 99452_at | Cluster Incl U49507: Mus musculus B6CBA Lisch7 mRNA, partial cds/cds = (0,1971)/gb = U49507/gi = 1236082/ | 559 | P | 293 | P | 378.3 | P | 426.8 |
| | | ug = Mm 4067/len = 2059/STRA = for | | | | | | | |
| 4 | 100886_f_at | Cluster Incl AI841415 UI-M-AH0-acx-c-02-0-UI s1 Mus musculus cDNA, 3 end/clone = UI-M-AH0-acx-c-02-0-UI/ | 276.2 | P | 261.9 | P | 275.2 | P | 330 |
| | | clone_end = 3/gb = AI841415/gi = 5475628/ug = Mm.5381/len = 303/STRA = for | | | | | | | |
| 4 | 100924_at | Cluster Incl X55123 GATA-binding protein 3/cds = (207,1538)/gb = X55123/gi = 51052/ug = Mm 606/len = 2056/STRA = for | 2072.2 | P | 1658.1 | P | 1025 | P | 1557.3 |
| 4 | 101469_at | Cluster Incl AF009368 Mus musculus neural precursor cell expressed developmentally downregulated Nedd9 (Nedd9) | 199.1 | P | 190.3 | P | 179.8 | P | 158.4 |
| | | mRNA, complete cds/cds = (292,2793)/gb = AF009366/gi = 2454522/ug = Mm 8315/len = 2889/STRA = for | | | | | | | |
| 4 | 101929_at | Cluster Incl AI836322 UI-N-AQ0-aag-a-02-0-UI s2 Mus musculus cDNA, 3 end/clone = UI-M-AQ0-aag-a-02-0-UI/ | 471.2 | P | 257.7 | P | 264.8 | P | 304.6 |
| | | clone_end = 3/gb = AI836322/gi = 5470530/ug = Mm.939/len = 211/STRA = for | | | | | | | |
| 4 | 102280_at | Cluster Incl AB006758: Mus musculus Pcdh7 mRNA for BH-protocadherin-a, complete cds/cds = (1004,4213)/ | 326.1 | P | 226.7 | P | 255.7 | P | 293 |
| | | gb = AB006758/gi = 3513311/ug = Mm 11964/len = 5520/STRA = for | | | | | | | |
| 4 | 102863_at | Cluster Incl AI550530: vx21g12.x1 Mus musculus cDNA, 3 end/clone = IMAGE-1265158/clone_end = 3/gb = AI550530/ | 246.4 | P | 196.9 | P | 226.1 | P | 185.4 |
| | | gi = 4482893/ug = Mm 1468/len = 234/STRA = for | | | | | | | |
| 4 | 103243_at | Cluster Incl AF083876 Mus musculus XMP (Xmp) mRNA, complete cds/cds = (109,627)/gb = AF083876/gi = 3462841/ | 1428.9 | P | 1011.6 | P | 852.7 | P | 1140.8 |
| | | ug = Mm 20423/len = 747/STRA = for | | | | | | | |
| 4 | 103501_at | Cluster Incl U02098 Mus musculus Pur-alpha mRNA, complete cds/cds = (518,1483)/gb = U02098/gi = 404649/ug = Mm 2236/ | 563.5 | P | 456.4 | P | 211.4 | P | 479.7 |
| | | len = 1609/STRA = for | | | | | | | |
| 4 | 103762_at | Cluster Incl AI853340 UI-M-BH0-air-b-06-0-UI s1 Mus musculus cDNA, 3 end/clone = UI-M-BH0-air-b-06-0-UI/ | 453.3 | P | 463.8 | P | 393.4 | P | 457 |
| | | clone_end = 3/gb = AI853340/gi = 5497246/ug = Mm.24632/len = 461/STRA = for | | | | | | | |
| 4 | 104183_at | Cluster Incl AI529005.ud44a03 y1 Mus musculus cDNA, 5 end/clone = IMAGE-1448716/clone_end = 5/gb-AI529005/ | 187.3 | P | 216.3 | P | 152.2 | P | 212.2 |
| | | gi = 4443140/ug = Mm 27380/len = 460/STRA = for | | | | | | | |
| 4 | 104220_at | Cluster Incl AF010133 Mus musculus Smad6 mRNA, complete cds/cds = (0,1487)/gb = AF010133/gi = 2507639/ | 395.3 | P | 351.4 | P | 301.1 | P | 327 |
| | | ug = Mm.27935/len = 1488/STRA = for | | | | | | | |
| 4 | 92409_at | Cluster Incl D45210: Mouse mRNA for zinc finger protein, complete cds/cds = (590,1813)/gb = D45210/gi = 633989/ | 138.3 | P | 128.7 | P | 25.7 | P | 121.4 |
| | | ug = Mm.4106/len = 2849/STRA = for | | | | | | | |
| 4 | 92460_at | Cluster Incl AB022316: Mus musculus gene for semaphorin w/cds = (78,2411)/gb = AB022316/gi = 4589501/ug = Mm.42035/ | 1387.4 | P | 1182.7 | P | 1282.9 | P | 1324.6 |
| | | len = 4024/STRA = for | | | | | | | |
| 4 | 92888_s_at | Cluster Incl U34973: Mus musculus protein tyrosine phosphatase-like mRNA, unspliced c-terminal product and spliced c- | 375 | P | 338.3 | P | 235.5 | P | 330.9 |
| | | terminal and STYX, complete cds/cds = (255,872)/gb = U34973/gi = 1063624/ug = Mm 4755/len = 1254/STRA = for | | | | | | | |
| 4 | 92893_at | Cluster Incl D90173 Mouse mRNA for NFI-B protein, clone NFI-B2/cds = (111,1709)/gb = D90173/gi = 220518/ug = Mm 4771/ | 187.2 | P | 198.5 | P | 172.8 | P | 131.9 |
| | | len = 3296/STRA = for | | | | | | | |
| 4 | 93246_at | Cluster Incl AW260482: um80e10.x1 Mus musculus cDNA, 3 end/clone = IMAGE-2317674/clone_end = 3/gb = AW260482/ | 454.3 | P | 225.9 | P | 163.6 | P | 421.3 |
| | | gi = 6633463/ug = Mm 6043/len = 635/STRA = rev | | | | | | | |
| 4 | 93439_f_at | Cluster Incl AA260005 va87f08.r1 Mus musculus cDNA, 5 end/clone = IMAGE-746435/clone_end = 5/gb = AA260005/ | 311 | P | 315.4 | P | 317.8 | P | 273.9 |
| | | gi = 1896491/ug = Mm.6617/len = 271/STRA = for | | | | | | | |
| 4 | 93667_at | Cluster Incl AW120511 UI-M-BH1-ana-a-04-0-UI s2 Mus musculus cDNA, 3 end/clone = UI-M-BH1-ana-a-04-0-UI/ | 165 | P | 136.9 | P | 136.3 | P | 169.4 |
| | | clone_end = 3/gb = AW120511/gi = 6095844/ug = Mm.80527/len = 454/STRA = for | | | | | | | |
| 4 | 97767_at | Cluster Incl X75649 M musculus mRNA for mouse hair acidic 2/cds = (28,1251)/gb = X75649/gi = 498977/ug = Mm 4161/ | 1707.6 | P | 888.4 | P | 954.5 | P | 1253.2 |
| | | len = 1591/STRA = for | | | | | | | |
| 4 | 99328_at | Cluster Incl U79738 Mus musculus homeodomain protein Dix-3 mRNA, parital cds/cds = (0,695)/gb = U79738/gi = 1718494/ | 1043.8 | P | 493.9 | P | 612.8 | P | 561.8 |
| | | ug = Mm.5194/len = 2221/STRA = for | | | | | | | |
| 4 | 100323_at | Cluster Incl Z23077 S-adenosylmethionine decarboxylase 3/cds = (0,1004)/gb = Z23077/gi = 396140/ug = Mm 87749/ | 604.2 | P | 351.7 | P | 238.1 | P | 554.8 |
| | | len = 1005/STRA = for | | | | | | | |
| 4 | 100324_g_at | Cluster Incl Z23077 S-adenosylmethionine decarboxylase 3/cds = (0,1004)/gb = Z23077/gi = 396140/ug = Mm 87749/ | 185 | P | 155.6 | P | 94.3 | P | 164.8 |
| | | len = 1005/STRA = for | | | | | | | |
| 4 | 101254_at | Cluster Incl L32751 Lipopolysacchande response/cds = (25,675)/gb = L32751/gi = 727168/ug = Mm 7521/len = 994/STRA = for | 5852.7 | P | 5580.7 | P | 4675.3 | P | 5727.3 |
| 4 | 102099_f_at | Cluster Incl AI843637.UI-M-AO1-aen-g-04-0-UI s1 Mus musculus cDNA, 3 end/clone = UI-M-AO1-aen-g-04-0-UI/ | 586.3 | P | 618.5 | P | 490.2 | P | 567.7 |
| | | clone_end = 3/gb = AI843637/gi = 5477850/ug = Mm 42435/len = 238/STRA = rev | | | | | | | |
| 4 | 102103_f_at | Cluster Incl AI482454 vg40d04 x1 Mus musculus cDNA, 3 end/clone = IMAGE-863815/clone_end = 3/gb = AI482454/ | 2028.4 | P | 1986.3 | P | 1582.9 | P | 1727.4 |
| | | gi = 4375680/ug = Mm 42484/len = 454/STRA = rev | | | | | | | |
| 4 | 92614_at | Cluster Incl M60523.Inhibitor of DNA binding 3/cds = (57,416)/gb = M60523/gi = 193872/ug = Mm 110/len = 969/STRA = for | 482.1 | A | 323.5 | P | 252.1 | P | 331.4 |
| 4 | 93348_at | Cluster Incl AA760359 vv76a05 r1 Mus musculus cDNA, 5 end/clone = IMAGE-1228304/clone_end = 5/gb = AA760359/ | 206.4 | P | 157.3 | P | 152.6 | P | 151.4 |
| | | gi = 2808153/ug = Mm 18803/len = 373/STRA = for | | | | | | | |
| 4 | 97814_at | Cluster Incl x75650 M musculus mRNA for mouse hair acidic 3/cds = (0,971)/gb = X75650/gi = 498975/ug = Mm.28178/ | 1380.8 | P | 528.2 | P | 281 | A | 533.9 |
| | | len = 1304/STRA = for | | | | | | | |
| 4 | 95649_at | Cluster Incl AI844440 UI-M-AJ1-ahe-a-02-0-UI s1 Mus musculus cDNA, 3 end/clone = UI-M-AJ1-ahe-a-02-0-UI/ | 477.4 | P | 486.6 | P | 357 | P | 477.5 |
| | | clone_end = 3/gb = AI844440/gi = 5488346/ug = Mm 29503/len = 301/STRA = for | | | | | | | |
| 4 | 95677_at | Cluster Incl AA881621 vx20h04 r1 Mus musculus cDNA, 5 end/clone = IMAGE-1265047/clone_end = 5/gb = AA881621/ | 428.1 | P | 254.8 | P | 210.3 | P | 253.9 |
| | | gi = 2990931/ug = Mm 29606/len = 507/STRA = for | | | | | | | |
| 4 | 99644_at | Cluster Incl AI845855-UI-M-AO1-aeg-e-11-0-UI s1 Mus musculus cDNA, 3 end/clone = UI-M-AO1-aeg-e-11-0-UI/ | 624.6 | P | 691.8 | P | 482.9 | P | 551.3 |
| | | clone_end = 3/gb = AI845855/gi = 5489761/ug = Mm 43636/len = 407/STRA = for | | | | | | | |
| 4 | 103558_at | Cluster Incl M27734 Mouse acedic keratin type I mRNA, complete cds/cds = (69,1319)/gb = M27734/gi = 198585/ | 1517 | P | 436 | P | 290.1 | P | 514.7 |
| | | ug = Mm.19109/len = 1585/STRA = for | | | | | | | |
| 4 | 103803_at | Cluster Incl AB013849 Mus musculus mRNA for peptidylarginine deiminase type III, complete cds/cds = (42,2038)/ | 274.7 | P | 256.4 | P | 185.3 | P | 224.2 |
| | | gb = AB013849/gi = 3645569/ug = Mm.20851/len = 3068/STRA = for | | | | | | | |
| 4 | 104124_at | Cluster Incl AW123135 UI-M-BH2.1-apd-a-07-0-UI s1 Mus musculus cDNA, 3 end/clone = UI-M-BH2-apd-a-07-0-UI/ | 161.1 | P | 104.1 | P | 35.8 | P | 114.6 |
| | | clone_end = 3/gb = AW123135/gi = 6098665/ug = Mm 22028/len = 503/STRA = for | | | | | | | |
| 4 | 96138_at | Cluster Incl AA682129: vu13b07.r1 Mus musculus cDNA, 5 end/clone = IMAGE-1180501/clone_end = 5/gb = AA682129/ | 315.1 | A | 326.5 | P | 268.8 | P | 395.6 |
| | | gi = 2663161/ug = Mm 28173/len = 611/STRA = for | | | | | | | |
| 4 | 96822_at | Cluster Incl AA734426 vr90e10 r1 Mus musculus cDNA, 5 end/clone = IMAGE-1136010/clone_end = 5/gb = AA734428/ | 502.9 | P | 554.6 | P | 360.2 | P | 544.9 |
| | | gi = 2756095/ug = Mm 29383/len = 409/STRA = rev | | | | | | | |
| 4 | 97983_s_at | Cluster Incl D45903. Mus musculus mRNA for unc-18 homologue, complete cds/cds = (35,1819)/gb = D45903/gi = 1944321/ | 134.7 | P | 140.6 | P | 175.6 | P | 211 |
| | | ug = Mm.3129/len = 3412/STRA = for | | | | | | | |
| 4 | 99502_at | Cluster Incl U80078 Mus musculus transcription factor BFCOL1 mRNA, complete cds/cds = (86,2395)/gb = U80078/ | 776.6 | P | 433.6 | P | 360 | P | 547.4 |
| | | gi = 1724123/ug = Mm 417/len = 2426/STRA = for | | | | | | | |
| 4 | 100895_at | Cluster Incl AW049497: UI-M-BH1-anf-a-01-0-UI s1 Mus musculus cDNA, 3 end/clone = UI-M-BH1-anf-a-01-0-UI/ | 163 | P | 122.1 | P | −13 | M | 27.4 |
| | | clone_end = 3/gb = AW049497/gi = 5910026/ug = Mm 567/len = 291/STRA = for | | | | | | | |
| 4 | 101376_at | Cluster Incl D86419 Mus musculus mRNA for glycine tyrosine-rich hair keratin protein, complete cds/cds = (2,235)/ | 1592.2 | P | 439.5 | P | 287.8 | P | 573 |
| | | gb = D86419/gi = 1944392/ug = Mm 6973/len = 520/STRA = for | | | | | | | |
| 4 | 103791_at | Cluster Incl AW048763 UI-M-BH1-amy-h-02-0-UI s1 Mus musculus cDNA, 3 end/clone = UI-M-BH1-amy-h-02-0-UI/ | 218.2 | P | 59.5 | P | 0.4 | A | 80.9 |
| | | clone_end = 3/gb = AW048763/gi = 5909292/ug = Mm 24958/len = 589/STRA = for | | | | | | | |
| 4 | 103998_at | Cluster Incl AW122699 UI-M-BH2 2-aot-b-09-0-UI s1 Mus musculus cDNA, 3 end/clone = UI-M-BH2 2-aot-b-09-0-UI/ | 81.9 | P | 46.1 | P | 74.8 | P | 77.2 |
| | | clone_end = 3/gb = AW122699/gi = 6098229/ug = Mm 26532/len = 435/STRA = for | | | | | | | |
| 4 | 104242_f_at | Cluster Incl AI835622 UI-M-AI0-aap-f-12-0-UI s1 Mus musculus cDNA, 3 end/clone = UI-M-AI0-aap-f-12-0-UI/clone_end = 3/ | 1362.1 | P | 1215.2 | A | 1062 | P | 1211.7 |
| | | gb = AI835622/gi = 5469835/ug = Mm.2821/len = 344/STRA = for | | | | | | | |
| 4 | 104643_at | Cluster Incl AI850848 UI-M-BH0-ajt-b-11-0-UI s1 Mus musculus cDNA, 3 end/clone = UI-M-BH0-ajt-b-11-0-UI/clone_end = 3/ | 388.5 | P | 257.7 | P | 241 | P | 288.5 |
| | | gb = AI850846/gi = 5494752/ug = Mm 31267/len = 341/STRA = for | | | | | | | |
| 4 | 104688_at | Cluster Incl Y09010 M musculus mRNA for serine/threonine kinase/cds = (138,2621)/gb = Y09010/gi = 1654011/ | 217 | P | 194.3 | P | 10.5 | P | 160.5 |
| | | ug = Mm 3313/len = 2732/STRA = for | | | | | | | |
| 4 | 104696_at | Cluster Incl AJ009840 Mus musculus cathepsin E gene, exon 1, partial/cds = (0,1193)/gb = AJ009840/gi = 3392946/ | 196.6 | P | 103.5 | P | 35.9 | P | 127.2 |
| | | ug = Mm 33671/len = 1194/STRA = for | | | | | | | |
| 4 | 93720_at | Cluster Incl AB005623: Mus musculus mRNA for 1-acyl-sn-glycerol-3-phosphate acyltransferase, complete cds/ | 253.1 | P | −18.7 | P | 97 | P | 130 |
| | | cds = (318,1175)/gb = AB005623/gi = 2457309/ug = Mm.8684/len = 1256/STRA = for | | | | | | | |
| 4 | 93935_at | Cluster Incl AF036332: Mus musculus nemo-like kinase (NIk) mRNA, complete cds/cds = (322,1869)/gb = AF036332/ | 91 | A | 74.3 | P | 47 | P | 51.9 |
| | | gi = 2828007/ug = Mm.9001/len = 2603/STRA = for | | | | | | | |
| 4 | 98340_at | Cluster Incl AA407332: EST00652 Mus musculus cDNA, 5 end/clone = C0001A09/clone_end = 5/gb = AA407332/ | 77.7 | P | 72.7 | P | 57.6 | P | 56.7 |
| | | gi = 2065567/ug = Mm 43174/len = 365/STRA = rev | | | | | | | |
| 4 | 100381_at | Cluster Incl M12347: Actin, alpha 1, skeletal muscle/cds = (70,1203)/gb = M1234/gi = 191572/ug = Mm.89137/len = 1446/ | 4408 | P | 2516.2 | P | 2955.1 | P | 3253.4 |
| | | STRA = for | | | | | | | |
| 4 | AFFX-BioB- | E Coli BioB 3 prime | 346.3 | A | 457.8 | P | 313.1 | P | 330.7 |
| | 3_at | | | | | | | | |
| 4 | 93046_at | Cluster Incl AW045233 UI-M-BH1-alh-f-10-0-UI s1 Mus musculus cDNA, 3 end/clone = UI-M-BH1-alh-f-10-0-UI/ | 179 | P | 147.4 | P | 107.4 | P | 144.8 |
| | | clone_end = 3/gb = AW045233/gi = 5905762/ug = Mm 1521/len = 412/STRA = for | | | | | | | |
| 4 | 93971_f_at | Cluster Incl AI838669 UI-M-AO0-aca-e-04-0-UI s1 Mus musculus cDNA, 3 end/clone = UI-M-AO0-aca-e-04-0-UI/ | 364.3 | P | 404.1 | P | 358.2 | P | 371.2 |
| | | clone_end = 3/gb = AI838669/gi = 5472882/ug = Mm 21667/len = 452/STRA = for | | | | | | | |
| 4 | 94064_at | Cluster Incl U05343: Mus musculus Balb/c zinc finger protein PZF (Pzf) mRNA, complete cds/cds = (137,1504)/gb = U05343/ | 297.5 | A | 230.7 | P | 256.8 | P | 222.2 |
| | | gi = 453375/ug = Mm 22066/len = 1514/STRA = for | | | | | | | |
| 4 | 94804_at | Cluster Incl L27453: Pre B-cell leukemia transcription factor 1/cds = (44,1087)/gb = L27453/gi = 456108/ug = Mm 22675/ | 172.1 | P | 86.5 | A | 91.2 | P | 164.6 |
| | | len = 1634/STRA = for | | | | | | | |
| 4 | 94493_at | Cluster Incl AF095905: Mus musculus CPETR2 (Cpetr2) mRNA, complete cds/cds = (55,714)/gb = AF095905/gi = 4262394/ | 201.5 | P | 139.5 | P | 124.9 | A | 161.6 |
| | | ug = Mm 28921/len = 1090/STRA = for | | | | | | | |
| 4 | 95658_at | Cluster Incl AW061085 UI-M-BH1-anp-c-04-0-UI s1 Mus musculus cDNA, 3 end/clone = UI-M-BH1-anp-c-04-0-UI/ | 145.9 | P | 85.6 | P | 86.5 | A | 62.5 |
| | | clone_end = 3/gb = AW061085/gi = 6008836/ug = Mm 29542/len = 464/STRA = for | | | | | | | |
| 4 | 96918_at | Cluster Incl AI790931 uk29g y1 Mus musculus cDNA, 5 end/clone = IMAGE-1970450/clone_end = 5/gb = AI790931/ | 1112.6 | P | 381.3 | P | 183 | P | 428.5 |
| | | gi = 5338647/ug = Mm 30154/len = 660/STRA = for | | | | | | | |
| 4 | 102415_r_at | Cluster Incl U28423 Mus musculus protein kinease inhibitor p58 mRNA, complete cds/cds = (100,1614)/gb = U28423/ | 195.9 | P | 126.8 | P | 104.3 | P | 182.1 |
| | | gi = 1353271/ug = Mm 12616/len = 1615/STRA = for | | | | | | | |
| 4 | 104096_at | Cluster Incl Y16386: Mus musculus mRNA for origin recognition complex subunit 4/cds = (0,1301)/gb = Y16386/gi = 3288543/ | 134.1 | P | 93.8 | P | 31.7 | A | 94.9 |
| | | ug = Mm 21834/len = 1302/STRA = for | | | | | | | |
| 4 | 104626_at | Cluster Incl AI787137 ui85c11.y1 Mus musculus cDNA, 5 end/clone = IMAGE-1889204/clone_end = 5/gb = AI787137/ | 167.3 | P | 62.6 | P | 160.8 | P | 89.4 |
| | | gi = 5334853/ug = Mm 24277/len = 297/STRA = for | | | | | | | |
| 4 | 96177_at | Cluster Incl AI854590 UI-M-BH0-ake-b-04-0-UI s1 Mus musculus cDNA, 3 end/clone = UI-M-BH0-ake-b-04-0-UI/ | 213.2 | P | 13.1 | A | −48.6 | P | 173.1 |
| | | clone_end = 3/gb = AI854590/gi = 549896/ug = Mm.28412/len = 435/STRA = for | | | | | | | |
| 4 | 98407_at | Cluster Incl U07602 Ephnn B1/cds = (1643,2680)/gb = U07602/gi = 2182217/ug = Mm 3374/len = 2692/STRA = rev | 311.1 | P | 180.2 | P | 122.1 | M | 316.3 |
| 4 | 102308_at | Cluster Incl AF045582: Mus musculus tubby protein 3 (Tulp3) mRNA, complete cds/cds = (26,1408)/gb = AF045582/ | 504.9 | P | 502.3 | P | 442.3 | P | 436.4 |
| | | gi = 3372490/ug = Mm 12887/len = 1508/STRA = for | | | | | | | |
| 4 | 103552_at | Cluster Incl AW046470 UI-M-BH1-akp-h-12-0-UI s1 Mus musculus cDNA, 3 end/clone = UI-M-BH1-akp-h-12-0-UI/ | 148 | P | 117.3 | P | 148.4 | P | 89.5 |
| | | clone_end = 3/gb = AW046470/gi = 5906999/ug = Mm 23822/len = 449/STRA = rev | | | | | | | |
| 4 | 95801_s_at | Cluster Incl L36316: Zinc finger protein 63/cds = (0,556)/gb = L36316/gi = 538414/ug = Mm 87838/len = 858/STRA = for | 199.7 | P | 141.4 | P | 69.5 | P | 122.8 |
| 4 | 93981_at | Cluster Incl J03520 Plasminogen acitvator, tissue/cds = (93,1772)/gb = J03520/gi = 202109/ug = Mm.21702/len = 2519/ | 561.9 | P | 270.7 | P | 163.9 | P | 379.2 |
| | | STRA = for | | | | | | | |
| 4 | 94222_at | Cluster Incl AI838737 UI-M-AO0-acf-d-01-0-UI.s1 Mus musculus cDNA, 3 end/clone = UI-M-AO0-acf-d-01-0-UI/ | 646.1 | P | 615.5 | P | 553 | P | 581.7 |
| | | clone_end = 3/gb = AI838737/gi = 5472950/ug = Mm 22248/len = 399/STRA = for | | | | | | | |
| 4 | 94916_at | Cluster Incl AW122260 UI-M-BH2 2-aov-g-11-0-UI s1 Mus musculus cDNA, 3 end/clone = UI-M-BH2 2-aov-g-11-0-UI/ | 459.8 | P | 474 | P | 210.7 | P | 368.6 |
| | | clone_end = 3/gb = AW122260/gi = 6097729/ug = Mm 24155/len = 161/STRA = for | | | | | | | |
| 4 | 96294_s_at | Cluster Incl AI854235 UI-M-BH0-ajz-g-07-0-UI s1 Mus musculus cDNA, 3 end/clone = UI-M-BH0-ajz-g-07-0-UI/ | 252 | P | 145.7 | P | 95.1 | P | 148.9 |
| | | clone_end = 3/gb = AI854235/gi = 5498141/ug = Mm 29898/len = 519/STRA = for | | | | | | | |
| 4 | 100607_at | Cluster Incl AF026124: Mus musculus schwannoma-associated protein (SAM9) mRNA, complete cds/cds = (417,1883)/ | 495.7 | A | 166 | P | 284.9 | P | 508.1 |
| | | gb = AF026124/gi = 2565395/ug = Mm 6483/len = 2248/STRA = for | | | | | | | |
| 4 | 102826_at | Cluster Incl J05663: Androgen regulated vas deferens protein/cds = (20,970)/gb = J05663/gi = 199897/ug = Mm.14460/ | 143.8 | P | 107 | P | 109.2 | P | 107.8 |
| | | len = 1225/STRA = for | | | | | | | |
| 4 | 103006_at | Cluster Incl AB012276 Mus musculus MRNA for ATFx, partial cds/cds = (0,254)/gb = AB012276/gi = 2988372/ug = Mm 1566/ | 382.1 | P | 362.3 | P | 275.8 | P | 413 |
| | | len = 850/STRA = for | | | | | | | |
| 4 | 103565_at | Cluster Incl AI843358 UI-M-AQ1-adz-d-04-0-UI s1 Mus musculus cDNA, 3 end/clone = UI-M-AQ1-adz-d-04-0-UI/ | 556.2 | P | 443.7 | P | 354.5 | M | 435.7 |
| | | clone_end = 3/gb = AI84358/gi = 5477571/ug = Mm.1915/len = 444/STRA = for | | | | | | | |
| 4 | 104346_at | Cluster Incl AI841914 UI-M-AO0-acd-f-08-0-UI.s1 Mus musculus cDNA, 3 end/clone = UI-M-AO0-acd-f-08-0-UI/ | 433.6 | P | 312.4 | P | 257.1 | A | 207 |
| | | clone_end = 3/gb = AI841914/gi = 5476127/ug = Mm.22537/len = 421/STRA = for | | | | | | | |
| 4 | 95596_at | Cluster Incl AW122957 UI-M-BH2 1-aoz-e-12-0-UI.s2 Mus musculus cDNA, 3 end/clone = UI-M-BH2 1-aoz-e-12-0-UI/ | 147.9 | P | 169.1 | P | 59.7 | A | 161.3 |
| | | clone_end = 3/gb = AW122957/gi = 6098487/ug = Mm.27918/len = 443/STRA = for | | | | | | | |
| 4 | 96735_at | Cluster Incl AW049732 UI-M-BH1-ani-g-05-0-UI.s1 Mus musculus cDNA, 3 end/clone = UI-M-BH1-ani-g-05-0-UI/ | 364.2 | P | 381.1 | P | 139.9 | M | 305.3 |
| | | clone_end = 3/gb = AW049732/gi = 5910261/ug = Mm.28896/len = 372/STRA = for | | | | | | | |
| 4 | 98998_r_at | Cluster Incl AF020790 Mus musculus hair keratin acidic 5 (Ha5) mRNA, complete cds/cds = (129,1406)/gb = AF020790/ | 847 | P | 335.1 | P | 110.8 | P | 342.9 |
| | | gi = 4103157/ug = Mm.37952/len = 1737/STRA = rev | | | | | | | |
| 4 | 102642_at | Cluster Incl L33415 Natural resistance-associated macrophage protein 2/cds = (60,1766)/gb = L33415/gi = 755040/ | 1321.9 | P | 1162.9 | P | 773.4 | M | 1280.7 |
| | | ug = Mm.1304/len = 2972/STRA = for | | | | | | | |
| 4 | 103257_at | Cluster Incl AA690483: vu52f06.r1 Mus musculus cDNA, 5 end/clone = IMAGE-1195043/clone_end = 5/gb = AA690483/ | 155.1 | P | 99.3 | P | 31.8 | P | 57.9 |
| | | gl = 2691419/ug = Mm.2073/len = 302/STRA = for | | | | | | | |
| 4 | 103965_at | Cluster Incl AI837032 UI-M-AP0-abj-d-02-0-UI s2 Mus musculus cDNA, 3 end/clone = UI-M-AP0-abj-d-02-0-UI/ | 327.5 | P | 111.4 | P | 45.7 | A | 282.8 |
| | | clone_end = 3/gb = AI837032/gi = 5471245/ug = Mm.25640/len = 289/STRA = for | | | | | | | |
| 4 | 98314_g_at | Cluster Incl AB025413.Mus musculus mRNA for Ten-m4, complete cds/cds = (187,8502)/gb = AB025413/gi = 4760781/ | 234 | P | 178.4 | P | 199 | P | 213.6 |
| | | ug = Mm.42204/len = 8585/STRA = for | | | | | | | |
| 4 | 100673_f_at | Cluster Incl U10091 Killer cell lectin-like receptor, subfamily A, member 5/cds = (108,908)/gb = U10091/gi = 533489/ | 34 | P | −15.3 | P | 45.3 | P | 46 |
| | | ug = Mm.14111/len = 1157/STRA = for | | | | | | | |
| 4 | 98881_at | Cluster Incl AI853675: UI-M-BH0-ajq-g-08-0-UI.s1 Mus musculus cDNA, 3 end/clone = UI-M-BH0-ajq-g-08-0-UI/ | 129.3 | P | 129 | A | 117.2 | P | 108 |
| | | clone_end = 3/gb = AI853675/gi = 5497621/ug = Mm.28525/len = 528/STRA = for | | | | | | | |
| 4 | 101006_at | Cluster Incl M35797: T-complex protein 1, related sequence 1/cds = (0,852)/gb = M35797/gi = 975268/ug = Mm.6797/ | 717.8 | P | 613.4 | P | 364.5 | P | 608.6 |
| | | len = 1692/STRA = for | | | | | | | |
| 4 | 102989_at | Cluster Incl U07808: Metallothionein 4/cds = (21,209)/gb = U07808/gi = 466268/ug = Mm 1536/len = 372/STRA = for | 417.2 | P | 200.2 | P | 27.6 | A | 186 |
| 4 | 103805_at | Cluster Incl AF092840: Mus musculus nibnn mRNA, complete cds/cds = (90,2345)/gb = AF092840/gi = 3676834/ | 192.5 | P | 184.4 | P | 120.7 | P | 102.2 |
| | | ug = Mm 20866/len = 2473/STRA = for | | | | | | | |
| 4 | 104364_at | Cluster Incl AF039840 Mus musculus mitogen-activated protein kinase-activated protein kinase MRNA, complete cds/ | 16.9 | P | 57.8 | A | 57.8 | P | 84.9 |
| | | cds = (706,2127)/gb = AF039840/gi = 2911812/ug = Mm 22612/len = 2243/STRA = for | | | | | | | |
| 4 | 95556_at | Cluster Incl AI841364: UI-M-AM0-adv-f-01-0-UI s1 Mus musculus cDNA, 3 end/clone = UI-M-AM0-adv-f-01-0-UI/ | 94.4 | M | −13.6 | M | 16.8 | P | 11.1 |
| | | clone_end = 3/gb = AI841364/gi = 5475577/ug = Mm 27754/len = 389/STRA = for | | | | | | | |
| 4 | 97343_at | Cluster Incl AI852735 UI-M-BH0-aji-h-03-0-UI s1 Mus musculus cDNA, 3 end/clone = UI-M-BH0-aji-h-03-0-UI/clone_end = 3/ | 509.7 | P | 434.7 | P | 313.8 | A | 708.8 |
| | | gb = AI852735/gi = 5496641/ug = Mm 29604/len = 292/STRA = rev | | | | | | | |
| 4 | 98429_at | Cluster Incl AB009653: Mus musculus mRNA for lysophospholipase II, complete cds/cds = (105,800)/gb = AB009653/ | 723.5 | P | 443.3 | M | 407 | A | 566.3 |
| | | gi = 4589452/ug = Mm 34302/len = 1593/STRA = for | | | | | | | |
| 4 | 98488_at | Cluster Incl AJ223361: Mus musculus mRNA for myosin heavy chain 28, partial/cds = (0,93)/gb = AJ223361/gi = 4582681/ | 97.6 | P | 55.4 | A | 49.9 | A | 78.2 |
| | | ug = Mm 35531/len = 199/STRA = for | | | | | | | |
| 4 | 101869_s_at | Cluster Incl J00413 Hemoglobin, beta adult major chain/cds = (0,443)/gb = J00413/gi = 193793/ug = Mm 87778/len = 444/ | 4270.7 | P | 1565.6 | A | 1362.6 | A | 3800.5 |
| | | STRA = for | | | | | | | |
| 4 | 103449_at | Cluster Incl AA795923 vu05110 r1 Mus musculus cDNA, 5 end/clone = IMAGE-1179787/clone_end = 5/gb = AA795923/ | 338.1 | P | 355 | P | 348.2 | A | 338.7 |
| | | gi = 2858878/ug = Mm 21577/len = 558/STRA = for | | | | | | | |
| 4 | 104157_at | Cluster Incl AW125643: UI-M-BH2.2.aqi-a-09-0-UI s1 Mus musculus cDNA, 3 end/clone = UI-M-BH2 2-aqi-a-09-0-UI/ | 113.5 | P | 55.9 | P | 8.9 | P | 73.7 |
| | | clone_end = 3/gb = AW125643/gi = 6101173/ug = Mm 27090/len = 462/STRA = rev | | | | | | | |
| 4 | 93901_at | Cluster Incl U78315 Pale ear/cds = (139,2250)/gb = U78315/gi = 2190591/ug = Mm 89325/len = 2687/STRA = for | 288.5 | P | 299.9 | P | 189.1 | P | 258.8 |
| 4 | 94781_at | Cluster Incl V00714: Hemoglobin alpha, adult chain 1/cds = (33,461)/gb = V00714/gi = 49899/ug = Mm 16820/len = 564/ | 3581.5 | P | 1760.1 | A | 1589.9 | A | 3413.5 |
| | | STRA = for | | | | | | | |
| 4 | 98330_at | Cluster Incl D70849: Zinc finger protein of the crebellum 3/cds = (538,1938)/gb = D70849/gi = 1345414/ug = Mm 4265/ | 214.4 | P | 145.6 | P | 144.2 | A | 156.8 |
| | | len = 3509/STRA = for | | | | | | | |
| 4 | 95858_at | Cluster Incl C76988 C76988 Mus musculus cDNA, 3 end/clone = J0023H02/clone_end = 3/gb = C76988/gi = 2517318/ | 154.2 | P | 109.1 | P | 60.8 | A | 140 |
| | | ug = Mm 24929/len = 594/STRA = rev | | | | | | | |
| 4 | 93556_at | Cluster Incl X99143: M musculus mRNA for hair keratin, mHb6/cds = (0,1370)/gb = X99143/gi = 1903237/ug = Mm 20285/ | 759.6 | P | 388.3 | P | 315.1 | A | 571.3 |
| | | len = 1808/STRA = for | | | | | | | |
| 4 | 94828_at | Cluster Incl AF004927 Opioid receptor, sigma 1/cds = (61,732)/gb = AF004927/gi = 3493173/ug = Mm 22745/len = 1618/ | 524.8 | P | 334.2 | P | 157.9 | A | 522.7 |
| | | STRA = for | | | | | | | |
| 4 | 103833_at | Cluster Incl AF077659 Mus musculus homeodomain-interacting protein kinase 2 mRNA, complete cds/cds = (157,3726)/ | 247.6 | P | 161.9 | A | 206.6 | P | 159.9 |
| | | gb = AF077659/gi = 3702957/ug = Mm 20934/len = 3964/STRA = for | | | | | | | |
| 4 | 98430_at | Cluster Incl X85169 Surfeit gene 5/cds = (188,610)/gb = X85169/gi = 1150511/ug = Mm 3434/len = 3424/STRA = for | 335.4 | P | 42.6 | P | −24.6 | M | 56.3 |
| 4 | 99521_at | Cluster Incl AB020239 Mus musculus mRNA for adenytate kinase 3, complete cds/cds = (82,753)/gb = AB020239/ | 82.5 | P | 96.4 | P | 59.1 | A | 13 |
| | | gi = 4760605/ug = Mm 42040/len = 916/STRA = for | | | | | | | |
| 4 | 101863_at | Cluster Incl C78246 C78246 Mus musculus cDNA, 3 end/clone = J0044H11/clone_end = 3/gb = C78246/gi = 2518576/ | 86.8 | P | 42.6 | A | 53.2 | A | 52.7 |
| | | ug = Mm 87617/len = 546/STRA = rev | | | | | | | |
| 4 | 102732_at | Cluster Incl AI849746: UI-M-AL1-ahn-d-09-0-UI s1 Mus musculus cDNA, 3 end/clone = UI-M-AL1-ahn-d-09-0-UI/ | 125.5 | A | 148.3 | P | 101.5 | A | 119.8 |
| | | clone_end = 3/gb = AI849746/gi = 5493652/ug = Mm 14458/len = 319/STRA = for | | | | | | | |
| 4 | 103447_at | Cluster Incl AI835989.UI-M-AJ0-abb-a-04-0-UI s1 Mus musculus cDNA, 3 end/clone = UI-M-AJ0-abb-a-04-0-UI/ | 142.5 | P | 125.7 | A | 48.5 | A | 127.3 |
| | | clone_end = 3/gb = AI835989/gi = 5470242/ug = Mm 21557/len = 316/STRA = for | | | | | | | |
| 4 | 93953_at | Cluster Incl D89871 Mus musculus mRNA for serine protease, complete cds/cds = (244,2529)/gb = D89871/gi = 2696702/ | 203.8 | A | 178.9 | A | 51.3 | A | 264.2 |
| | | ug = Mm 9431/len = 2614/STRA = for | | | | | | | |
| 4 | 94712_at | Cluster Incl U73620 Vascular endothelial growth factor C/cds = (153,1400)/gb = U73620/gi = 1778356/ug = Mm 1402/ | 20.1 | P | 3.8 | A | 0.1 | A | 16.1 |
| | | len = 1818/STRA = for | | | | | | | |
| 4 | 99681_at | Cluster Incl T25554: EST00587 Mus musculus cDNA, 3 end/clone = B0001C09/clone_end = 3/gb = T25554/gi = 562875/ | 153.2 | P | 135.4 | P | 137.2 | P | 117.9 |
| | | ug = Mm 23757/len = 275/STRA = rev | | | | | | | |
| 4 | 93022_i_at | Cluster Incl M32459: Mouse histone H3 (H3 2-221) gene, complete cds/cds = (21,431)/gb = M32459/gi = 193855/ | 47 | A | 45.6 | A | 35.2 | A | 55.6 |
| | | ug = Mm 14775/len = 595/STRA = for | | | | | | | |
| 4 | 93594_r_at | Cluster Incl U87948 Epithelial membrane protein 3/cds = (201,692)/gb = U87948/gi = 2358253/ug = Mm 20829/len = 695/ | 592.8 | M | 357.8 | P | 398 | A | 396.2 |
| | | STRA = for | | | | | | | |
| 4 | 99080_at | Cluster Incl AI853930 UI-M-BH0-aju-g-05-0-UI s1 Mus musculus cDNA, 3 end/clone = UI-M-BH0-aju-g-05-0-UI/ | 201.6 | P | 168.9 | A | 231.5 | A | 191 |
| | | clone_end = 3/gb = AI853930/gi = 5497836/ug = Mm 3886/len = 442/STRA = for | | | | | | | |
| 4 | 103595_at | Cluster Incl AW012359 uo03h08 y1 Mus musculus cDNA, 5 end/clone = IMAGE-2582367/clone_end = 5/gb = AW012359/ | 286.6 | P | 139 | A | 198.8 | A | 201 |
| | | gi = 5861137/ug = Mm 19974/len = 537/STRA = for | | | | | | | |
| 4 | 104767_f_at | Cluster Incl AW060921.UI-M-BH1-anv-c-12-0-UI s1 Mus musculus cDNA, 3 end/clone = UI-M-BH1-anv-c-12-0-UI/ | 341.7 | M | 339.6 | P | 320.6 | P | 382.9 |
| | | clone_end = 3/gb = AW060921/gi = 6008672/ug = Mm 24875/len = 438/STRA = for | | | | | | | |
| 4 | 96775_at | Cluster Incl X56690: Chromobox homolog (Drosophila HP1 beta)/cds = (0,557)/gb = X56690/gi = 53162/ug = Mm.29055/ | 240 | A | 199.9 | A | 136.6 | A | 188 |
| | | len = 558/STRA = for | | | | | | | |
| 4 | 97997_at | Cluster Inci U88566 Mus musculus secreted frizzled related protein sFRP-1 (Sfrp1) mRNA, complete cds/cds = (256,1200)/ | 110.6 | A | 71.5 | M | 85.5 | A | 178.6 |
| | | gb = U88566/gi = 1946340/ug = Mm 3171/len = 2659/STRA = for | | | | | | | |
| 4 | 102192_r_at | Cluster Incl AF068246.Mus musculus SA protein mRNA, complete cds/cds = (274,2010)/gb = AF068246/gi = 3928675/ | 70.8 | P | 23.3 | A | 19.5 | A | 34.1 |
| | | ug = Mm 9821/len = 2544/STRA = rev | | | | | | | |
| 4 | 102652_at | Cluster Incl X56959 POU domain, class 3, transcription factor 1/cds = (35,1384)/gb = X56959/gi = 49681/ug = Mm 1330/ | 271.2 | M | 275.7 | A | 179.2 | A | 337.6 |
| | | len = 1516/STRA = for | | | | | | | |
| 4 | 103225_at | Cluster Incl AW018420: ul95h03 x1 Mus musculus cDNA, 3 end/clone = IMAGE-21598614/clone_end = 3/gb = AW018420/ | 210.6 | P | 165.9 | P | 175.3 | P | 120.4 |
| | | gi = 5871950/ug = Mm 20169/len/STRA = for | | | | | | | |
| 4 | 92913_at | Cluster Incl Z48670: M. musculus mRNA for ALDR/cds = (203,2428)/gb = Z48670/gi = 1107731/ug = Mm.4817/len = 2531/ | 235.4 | P | 125.7 | A | −27.8 | A | 91.4 |
| | | STRA = for | | | | | | | |
| 4 | 93634_at | Cluster Incl AW125157: UI-M-BH2-1-apz-d-12-0-UI s1 Mus musculus cDNA, 3 end/clone = UI-M-BH2-1-apz-12-0-UI/ | 584.3 | P | 483.4 | A | 397.5 | P | 504.4 |
| | | clone_end = 3/gb = AW125157/gi = 6100687/ug = Mm 76794/len = 484/STRA = for | | | | | | | |
| 4 | 93889_f_at | Cluster Incl X90778: M. musculus H2B gene/cds = (0,383)/gb = X90778/gi = 1518337/ug = Mm 89080/len = 384/STRA = rev | 39.4 | A | 50.6 | A | 54.7 | A | 61.5 |
| 4 | 95904_at | Cluster Incl AI835760 UI-M-Al0-aan-h-04-0-UI.s1 Mus musculus cDNA, 3 end/clone = UI-M-Al0-aan-h-04-0-UI/ | 404.9 | M | 304.6 | P | 372.7 | P | 376.2 |
| | | clone_end = 3/gb = AI835760/gi = 5469973/ug = Mm 24347/len = 490/STRA = for | | | | | | | |
| 4 | 97703_at | Cluster Incl AI427631 mj85e04 x1 Mus musculus cDNA, 3 end/clone = IMAGE-482910/clone_end = 3/gb = AI427631/ | −6.8 | A | −6.8 | P | −5.6 | A | −18 |
| | | gi = 4273557/ug = Mm 38034/len = 290/STRA = rev | | | | | | | |
| 5 | 92568_at | Cluster Incl M74555 House-keeping protein 1/cds = (87,1277)/gb = M74555/gi = 193870/ug = Mm 103/len = 2415/STRA = for | 157.5 | P | 227.6 | P | 212.3 | P | 121.2 |
| 5 | 92581_at | Cluster Incl U07159 Acetyl coenzyme A dehydrogenase, medium chain/cds = (7,1272)/gb = U07159/gi = 463908/ | 854.6 | P | 618.7 | P | 494.1 | P | 384.6 |
| | | ug = Mm 10530/len = 1846/STRA = for | | | | | | | |
| 5 | 92640_at | Cluster Incl U47024: Materrial embryonic message 3/cds = (137,2401)/gb = U47024/gi = 1354049/ug = Mm 1178/len = 3128/ | 965.1 | P | 1029.6 | P | 929.1 | P | 857.8 |
| | | STRA = for | | | | | | | |
| 5 | 92773_at | Cluster Incl AF079528 Mus musculus IER5 (ler5) gene, complete cds/cds = (0,926)/gb = AF079528/gi = 3386543/ug = | 197.4 | P | 230.7 | P | 155.1 | P | 252.1 |
| | | Mm 12246/len = 927/STRA = for | | | | | | | |
| 5 | 92789_f_at | Cluster Incl Y12474 M.musculous mRNA for centrin gene/cds = (0,503)/gb = Y12474/gi = 2246423/ug = Mm.12481/len = 504 | 394.5 | P | 310.4 | P | 262.6 | P | 277.9 |
| | | STRA = for | | | | | | | |
| 5 | 92818_at | Cluster Incl AF077330 Mus musculus NEDD8-conjugating enzyme (Uba3) mRNA, complete cds/cds = (39,1364)/ | 334.9 | P | 275.1 | P | 170 | P | 203.2 |
| | | gb = AF077330/gi = 3335647/ug = Mm 12892/len = 2117/STRA = for | | | | | | | |
| 5 | 93025_at | Cluster Incl AI835257 UI-M-AQ0-aab-b-03-0-UI s1 Mus musculus cDNA, 3 end/clone = UI-M-AQ0-aab-b-03-0-UI/ | 1020.1 | P | 1091.5 | P | 1059.1 | P | 906.3 |
| | | clone_end = 3/gb = AI835257/gi = 5469470/ug = Mm 1479/len = 342/STRA = for | | | | | | | |
| 5 | 93081_at | Cluster Incl U35142: Mus mussculus retinoblastoma-binding protein (mRbAp46) mRNA, complete cds/cds = (259,1536)/ | 2298.7 | P | 2112.7 | P | 1968.4 | P | 1960.5 |
| | | gb = U35142/gi = 106276/ug = Mm.1603/len = 1748/STRA = for | | | | | | | |
| 5 | 93082_at | Cluster Incl AW124081 UI-M-BH2 1-apn-g-11-0-UI.s1 Mus musculus cDNA, 3 end/clone = UI-M-BH2 1-apn-g-11-0-UI/ | 1886.1 | P | 1867.1 | P | 2126.8 | P | 1353.2 |
| | | clone_end = 3/gb = AW124081/gi = 6099611/ug = Mm.1608/len = 417/STRA = for | | | | | | | |
| 5 | 93088_at | Cluster Incl X01838 Beta-2 microglobulin/cds = (52,411)/gb = X01838/gi = 50104/ug = Mm 163/len = 647/STRA = for | 1814.5 | P | 1037 | P | 662.7 | P | 1219.9 |
| 5 | 93089_at | Cluster Incl X12507 Eukaryotic translation initiation facto 4A2/cds = (36,1259)/gb = X12507/gi = 50822/ug = Mm 16323/ | 844.1 | P | 524.7 | P | 575.6 | P | 579 |
| | | len = 1894/STRA = for | | | | | | | |
| 5 | 93118_at | Cluster Incl AI844131 UI-M-AL1-ahj-d-09-0-UI s1 Mus musculus cDNA, 3 end/clone = UI = M-AL1-ahj-d-09-0-UI/ | 219.6 | P | 235.3 | P | 126.8 | P | 202.4 |
| | | clone_end = 3/gb = AI844131/gi = 5488037/ug Mm 16767/len = 425/STRA = for | | | | | | | |
| 5 | 93267_at | Cluster Incl AA688834: vs04a06 r1 Mus musculus cDNA, 5 end/clone = IMAGE-1137202/clone_end = 5/gb = AA688834/ | 686.6 | P | 598.3 | P | 386.9 | P | 478.9 |
| | | gi = 2678263/ug = Mm 17307/len = 514/STRA = for | | | | | | | |
| 5 | 93274_at | Cluster Incl M38381 CDC-like kinase/cds = (85,1536)/gb = M38381/gi = 201070/ug = Mm 1761/len = 1740/STRA = for | 385.8 | P | 139.5 | P | 186.3 | P | 132.7 |
| 5 | 93278_at | Cluster Incl M91458.Sterol carrier protein 2, liver/cds = (15,1658)/gb = M91458/gi = 293793/ug-Mm 1779/len = 2152/ | 728.9 | P | 727.9 | P | 502.9 | P | 479.7 |
| | | STRA = for | | | | | | | |
| 5 | 93337_at | Cluster Incl U10119: Mus musculus SKD1 protein mRNA, complete cds/cds = (229,1563)/gb = U10119/gi = 557875/ | 820.4 | P | 764.6 | P | 440.4 | P | 533.9 |
| | | ug = Mm 18705/len = 2844/STRA = for | | | | | | | |
| 5 | 93488_at | Cluster Incl AI840579 UI-M-AM0-adp-h-12-0-UI.s1 Mus musculus cDNA, 3 end/clone = UI-M-AM0-adp-h-12-0-UI/ | 225.7 | P | 226.8 | P | 177.1 | P | 196.5 |
| | | clone_end = 3/gb = AI840579/gi = 5474792/ug = Mm.19015/len = 272/STRA = for | | | | | | | |
| 5 | 93491_f_at | Cluster Incl AI836623 UI-M-AP0-abh-a-10-0-UI s1 Mus musculus cDNA, 3 end/clone = UI-M-AP0-abh-a-10-0-UI/ | 557.9 | P | 497.7 | P | 556.2 | P | 459 |
| | | clone_end = 3/gb = AI836623/gi = 5470836/ug = Mm 19060/len = 523/STRA = for | | | | | | | |
| 5 | 93493_at | Cluster Incl X65627 D-E-A-D (aspartate-glutamate-alanine-aspartate) box polypeptide 5/cds = (160,2004)/gb = X65627/ | 864.1 | P | 864.4 | P | 767.8 | P | 748.4 |
| | | gi = 51262/ug = Mm.19101/len = 2318/STRA = for | | | | | | | |
| 5 | 93501_f_at | Cluster Incl AF058955 Mus musculus ATP-specific succinyl-CoA synthetase beta subunit (Sca) mRNA, partial cds/ | 604.5 | P | 580 | P | 464 | P | 583.7 |
| | | cds = (0,1280)/gb = AF058955/gi = 3766200/ug = Mm.19154/len = 1991/STRA = for | | | | | | | |
| 5 | 93503_at | Cluster Incl U88567: Mus musculus secreted frizzled related protein saFRP-2 (Sfrp2) mRNA, complete cds/cds = (235,1122)/ | 438.7 | P | 625.3 | P | 521.6 | P | 322.7 |
| | | gb = U88567/gi = 1946342/ug = Mm 19155/len = 2028 STRA = for | | | | | | | |
| 5 | 93546_s_at | Cluster Incl D14572 Mouse mRNA for PEBP2b1 protein, complete cds/cds = (232,795)/gb = D14572/gi = 303693/ | 1319.4 | P | 1209.8 | P | 1143.2 | P | 954.2 |
| | | ug = Mm 2018/len = 2805/STRA = for | | | | | | | |
| 5 | 93572_at | Cluster Incl AI835051 UI-M-AI1-afn-e-02-0-UI s1 Mus musculus cDNA, 3 end/clone-UI-M-AI1-afn-o-02-0-UI/clone_end = 3/ | 678 | P | 631.1 | P | 562.7 | P | 614.4 |
| | | gb = AI835051/gi = 5469264/ug = Mm.2041/len = 180/STRA = for | | | | | | | |
| 5 | 93773_f_at | Cluster Incl AI835041 UI-M-AI1-afn-d-02-0-UI s1 Mus musculus cDNA, 3 end/clone = UI-M-AI1-afn-d-02-0-UI/clone_end = 3/ | 667.3 | P | 493.1 | P | 285.5 | P | 320.7 |
| | | gb = AF835041/gi = 5469254/ug = Mm 21203/len = 409/STRA = for | | | | | | | |
| 5 | 93784_at | Cluster Incl AB010828 Mus musculus mRNA for BCNT, complete cds/cds = (103,990)/gb = AB010828/gi = 3327263/ | 361.7 | P | 385.4 | P | 292.9 | P | 370.8 |
| | | ug = Mm.21310/len = 1215/STRA = for | | | | | | | |
| 5 | 93794_at | Cluster Incl AI846393 UI-M-AN1-afa-h-03-0-UI s1 Mus musculus cDNA, 3 end/clone = UI-M-AN1-afa-h-03-0-UI/ | 544.5 | P | 480.5 | P | 426.9 | P | 439.6 |
| | | clone_end = 3/gb = AI846393/gi = 5490299/ug = Mm 21396/len = 193/STRA = for | | | | | | | |
| 5 | 93806_at | Cluster Incl AI848671 UI-M-AH1-agv-e-02-0-UI s1 Mus musculus cDNA, 5 end/clone = UI-M-AH1-agv-e-20-0-UI/ | 516.9 | P | 449.2 | P | 254.6 | P | 165.8 |
| | | clone_end = 3/gb = AI848671/gi = 5492577/ug = Mm.21446/len = 385/STRA = for | | | | | | | |
| 5 | 93986_at | Cluster Incl AA939576: vz51e01 r1 Mus musculus cDNA, 5 end/clone = IMAGE-1330008/clone_end = 5/gb = AA939576/ | 532.5 | P | 518.4 | P | 514.2 | P | 466.6 |
| | | gi = 3100353/ug = Mm 21714/len = 400/STRA = for | | | | | | | |
| 5 | 93990_at | Cluster Incl Y14196 Mus musculus mRNA for heterogeneous nuclear ribonucleoprotein H/cds = (82,1431)/gb = Y14196/ | 1215.2 | P | 798.8 | P | 486.6 | P | 694.5 |
| | | gb = 2253040/ug = Mm.21740/len = 2181/STRA = for | | | | | | | |
| 5 | 94081_at | Cluster Incl AW124390 UI-M-BH2 1-ape-c-03-0-UI s1 Mus musculus cDNA, 3 end/clone = UI-M-BH2 1-ape-c-03-0-UI/ | 254.6 | P | 265.7 | P | 269.7 | P | 207.9 |
| | | clone_end = 3/gb = AW124390/gi = 6099920/ug = Mm 22162/len = 217/STRA = for | | | | | | | |
| 5 | 94250_at | Cluster Incl U14172 Mus musculus p162 protein mRNA, complete cds/cds = (126,4160)/gb = U14172/gi = 1205975/ | 589.2 | P | 440.1 | P | 203.8 | P | 269 |
| | | ug = Mm.2238/len = 5119/STRA = for | | | | | | | |
| 5 | 94319_at | Cluster Incl L04966 Mus musculus ras-related protein (rab18) mRNA, complete cds/cds = (4,624)/gb = L04966/gi = 388324/ | 1174.9 | P | 1110.7 | P | 906.4 | P | 964.9 |
| | | ug = Mm 22660/len = 692/STRA = for | | | | | | | |
| 5 | 94810_at | Cluster Incl X79233: Ewing sarcoma homolog/cds = (56,2023)/gb = X79233/gi = 488512/ug = Mm.22691/len = 2188/STRA = for | 857.3 | P | 876.3 | P | 1012.1 | P | 617.7 |
| 5 | 94830_at | Cluster Incl AI854300 UI-M-BH0-ajs-f-06-0-UI.s1 Mus musculus cDNA, 3 end/clone = UI-M-BHO-ajs-f-06-0-UI/clone_end = 3/ | 593.9 | P | 464.7 | P | 520.5 | P | 520.2 |
| | | gb = AI854300/gi = 5498206/ug = Mm.22749/len = 554/STRA = for | | | | | | | |
| 5 | 95418_at | Cluster Incl AI848851 UI-M-AJ1-ahb-d-09-0 UI s1 Mus musculus cDNA, 3 end/clone = UI-M-AJ1-ahb-d-09-0-UI/ | 166.8 | P | 311.5 | P | 188.6 | P | 145.3 |
| | | clone_end = 3/gb = AI848851/gi = 5492757/ug = Mm 24348/len = 377/STRA = for | | | | | | | |
| 5 | 95432_f_at | Cluster Incl AI844034 UI-M-AL1-ahi-d-02-0-UI s1 Mus musculus cDNA, 3 end/clone = UI-M-AL1-ahi-d-02-0-UI/ | 567.2 | P | 524 | P | 393.5 | P | 419.8 |
| | | clone_end = 3/gb = AI844034/gi = 5487940/ug = Mm 24463/len = 404/STRA = for | | | | | | | |
| 5 | 95456_r_at | Cluster Incl U41626 Split hand/foot deleted gene 1/cds = (46,258)/gb = U41626/gi = 1209725/ug = Mm 2469/len = 432/ | 1646.8 | P | 1769.3 | P | 1530.7 | P | 1442.2 |
| | | STRA = for | | | | | | | |
| 5 | 95472_f_at | Cluster Incl AI526902 uj47g07.x1 Mus musculus cDNA, 3 end/clone = IMAGE-1923132/clone_end = 3/gb = AI526902/ | 1173.3 | P | 1307.8 | P | 1173.9 | P | 1055.9 |
| | | gi = 4441037/ug = Mm 24805/len = 484/STRA = for | | | | | | | |
| 5 | 95485_at | Cluster Incl D29639 Mouse embryonal carcinoma cell mRNA for 3-hydroxyacyl CoA dehydrogenase, complete cds/ | 878.4 | P | 1481.7 | P | 1063.5 | P | 864.9 |
| | | cds = (53,964)/gb = D29639/gi = 1125025/ug = Mm 2491/len = 1679/STRA = for | | | | | | | |
| 5 | 95488_at | Cluster Incl AW045756 UI-M-BH1-akt-b-04-0-UI s1 Mus musculus cDNA, 3 end/clone = UI-M-BH1-akt-b-04-0-UI/ | 744.6 | P | 797.8 | P | 739.6 | P | 784.1 |
| | | clone_end = 3/gb = AW045756/gi = 5906285/ug = Mm.25018/len = 357/STRA = for | | | | | | | |
| 5 | 95516_at | Cluster Incl AB027290 Mus musculus SID 99 mRNA for small GTP binding protein, complete cds/cds = (215,820)/ | 953.7 | P | 784.6 | P | 618.1 | P | 620.8 |
| | | gb = AB027290/gi = 5931815/ug = Mm 25306/len = 1276/STRA = for | | | | | | | |
| 5 | 96029_at | Cluster Incl AI020542 ub24g02.r1 Mus musculus cDNA, 5 end/clone = IMAGE-1378706/clone_end = 5/gb = AI020542/ | 347.2 | P | 468.1 | P | 347.4 | P | 382.2 |
| | | gi = 3234878/ug = Mm 25779/len = 374/STRA = for | | | | | | | |
| 5 | 96096_f_at | Cluster Incl AI648018 uk39c03.x1 Mus musculus cDNA, 3 end/clone = IMAGE-1971364/clone_end = 3/gb = AI648018/ | 261.4 | P | 267.1 | P | 220.3 | P | 210.3 |
| | | gi = 4726696/ug = Mm 26772/len = 543/STRA = for | | | | | | | |
| 5 | 96112_at | Cluster Incl AI651178 UI-M-BH0-akg-g-09-UI s1 Mus musculus cDNA, 3 end/clone = UI-M-BH0-akg-g-09-0-UI/ | 1050.7 | P | 1044.3 | P | 920.1 | P | 775.5 |
| | | clone_end = 3/gb = AI851178/gi = 5495049/ug = Mm.26949/len = 515/STRA = for | | | | | | | |
| 5 | 96809_at | Cluster Incl AW122195 UI-M-BH2.2-aov-b-06-0-UI s1 Mus musculus cDNA, 3 end/clone = UI-M-BH2 2-aov-b-06-0-UI/ | 476.4 | P | 517.3 | P | 297 | P | 379.7 |
| | | clone_end = 3/gb = AW122195/gi = 6097650/ug = Mm 27074/len = 589/STRA = for | | | | | | | |
| 5 | 96651_at | Cluster Incl AF035263 Mus musculus BAF57 (Baf57) gene, complete cds/cds = (0,1235)/gb = AF035283/gi = 2614754/ | 227.5 | P | 245.6 | P | 158.3 | P | 199.4 |
| | | ug = Mm 27330/len = 1236/STRA = for | | | | | | | |
| 5 | 96665_at | Cluster Incl AW211618 uo84d01 y1 Mus musculus cDNA, 5 end/clone = IMAGE-2649217/clone_end = 5/gb = AW211618/ | 459.2 | P | 396.6 | P | 397.6 | P | 425.4 |
| | | gi = 6517659/ug = Mm 27373/len = 442/STRA = for | | | | | | | |
| 5 | 96688_at | Cluster Incl AI845463 UI-M-AO1-aej-g-06-0-UI.s1 Mus musculus cDNA, 3 end/clone = UI-M-AO1-aej-g-06-0-UI/ | 142.3 | P | 109.3 | P | 114.3 | P | 57.4 |
| | | clone_end = 3/gb = AI845463/gi = 5489369/ug = Mm 27487/len = 423/STRA = for | | | | | | | |
| 5 | 97255_at | Cluster Incl AW061318 UI-M-BH1-anw-e-02-0-UI s1 Mus musculus cDNA, 3 end/clone = UI-M-BH1-anw-e-02-0-UI/ | 210.3 | P | 378.3 | P | 264.8 | P | 179.7 |
| | | clone_end = 3/gb = AW061318/gi = 6009069/ug = Mm 27860/len = 402/STRA = for | | | | | | | |
| 5 | 97279_at | Cluster Incl AI837615 UI-M-AK0-adg-d-01-0-UI s1 Mus musculus cDNA, 3 end/clone = UI-M-AK0-adg-d-01-0-UI/ | 322.8 | P | 389.2 | P | 413.6 | P | 295.7 |
| | | clone_end = 3/gb = AI837615/gi = 5471828/ug = Mm 27959/len = 467/STRA = for | | | | | | | |
| 5 | 97302_at | Cluster Incl AI854285.UI-M-BH0-ajs-e-02-0-UI s1 Mus musculus cDNA, 3 end/clone = UI-M-BH0-ajs-e-02-0-UI/ | 462 | P | 382.2 | P | 358 | P | 263.4 |
| | | clone_end = 3/gb = AI854285/gi = 5498191/ug = Mm 28049/len = 427/STRA = for | | | | | | | |
| 5 | 97304_at | Cluster Incl AI836100: UI-M-AJ0-abb-h-02-0-UI s1 Mus musculus cDNA, 3 end/clone = UI-M-AJ0-abb-h-02-0-UI/ | 458.8 | P | 417.7 | P | 327.1 | P | 277.3 |
| | | clone_end = 3/gb = AI836100/gi = 5470313/ug = Mm 28052/len = 358/STRA = for | | | | | | | |
| 5 | 97812_at | Cluster Incl AF006465 Mus musculus B cell antigen receptor Ig beta associated protein 1 (IBAP-1) mRNA, complete cds/ | 194.4 | P | 159.4 | P | 257.5 | P | 115.5 |
| | | cds = (45,2006)/gb = AF006465/gi = 4101717/ug = Mm 28165/len = 2190/STRA = for | | | | | | | |
| 5 | 97838_at | Cluster Incl AA684508 vr02b07.r1 Mus musculus cDNA, 5 end/clone = IMAGE-1110613/clone_end = 5/gb = AA684508/ | 536.8 | P | 386.7 | P | 522.9 | P | 479.4 |
| | | gi = 2670225/ug = Mm 28239/len = 398/STRA = for | | | | | | | |
| 5 | 97839_at | Cluster Incl AI841960.UI-M-AH0-acu-c-10-0-UI.s2 Mus musculus cDNA, 3 end/clone = UI-M-AH0-acu-c-10-0-UI/ | 1640.7 | P | 1522 | P | 1453.4 | P | 1397.9 |
| | | clone_end = 3/gb = AI841960/gi = 5476173/ug = Mm 28240/len = 335/STRA = for | | | | | | | |
| 5 | 97880_at | Cluster Incl AW061024: UI-M-BH1-amn-g-03-0-UI s1 Mus musculus cDNA, 3 end/clone = UI-M-BH1-amn-g-03-0-UI/ | 504.8 | P | 610.8 | P | 605.2 | P | 608.3 |
| | | clone_end = 3/gb = AW061024/gi = 6008775/ug = Mm 28365/len = 457/STRA = for | | | | | | | |
| 5 | 97896_i_at | Cluster Incl AW125218: UI-M-BH2.1-aps-c-06-0-UI s1 Mus musculus cDNA, 3 end/clone = UI-M-BH2 1-aps-c-06-0-UI/ | 251 | P | 189.2 | P | 180.3 | P | 185.3 |
| | | clone_end = 3/gb = AW125218/gi = 6100748/ug = Mm 28421/len = 319/STRA = for | | | | | | | |
| 5 | 97897_at | Cluster Incl AI848435 UI-M-AH1-ags-e-03-0-UI s1 Mus musculus cDNA, 3 end/clone = UI-M-AH1-ags-e-03-0-UI/ | 432.1 | P | 442.9 | P | 468 | P | 393.6 |
| | | clone_end = 3/gb = AI848435/gi = 5492341/ug = Mm 28422/len = 500/STRA = for | | | | | | | |
| 5 | 98905_at | Cluster Incl AJ223782 Mus musculus CDC10 gene, promoter, exon 1 and joined CDS/cds = (68,1378)/gb = AJ223782/ | 1060.8 | P | 872 | P | 512.4 | P | 604.2 |
| | | gi = 2827748/ug = Mm 2858/len = 2392/STRA = for | | | | | | | |
| 5 | 98909_at | Cluster Incl AW060843: UI-M-BH1-amx-d-06-0-UI s1 Mus musculus cDNA, 3 end/clone = UI-M-BH1-amx-d-06-0-UI/ | 356.8 | P | 423.4 | P | 379.5 | P | 343.7 |
| | | clone_end = 3/gb = AW060843/gi = 6008594/ug = Mm 28591/len = 419/STRA = for | | | | | | | |
| 5 | 98946_at | Cluster Incl AF033166 Mus musculus WSB-1 mRNA, complete cds/cds = (0,1265)/gb = AF033186/gi = 2766488/ | 156.4 | P | 174.5 | P | 294.2 | P | 173.4 |
| | | ug = Mm 28719/len = 1266/STRA = for | | | | | | | |
| 5 | 94460_at | Cluster Incl AA691445 vs11h05.r1 Mus musculus cDNA, 5 end/clone = IMAGE-1137945/clone_end = 5/gb = AA691445/ | 100.7 | P | 221.6 | P | 99.4 | P | 89.6 |
| | | gi = 2692383/ug = Mm 28821/len = 500/STRA = for | | | | | | | |
| 5 | 94505_at | Cluster Incl AW124934 UI-M-BH2 1-apu-g-11-0-UI.s1 Mus musculus cDNA, 3 end/clone = UI-M-BH2.1-apu-g-11-0-UI/ | 490 | P | 530.5 | P | 376 | P | 389.9 |
| | | clone_end = 3/gb = AW124934/gi = 6100464/ug = Mm 28957/len = 425/STRA = for | | | | | | | |
| 5 | 94507_at | Cluster Incl U15977 Mus musculus long chain fatty acyl CoA synthetase mRNA, complete cds/cds = (0,2099)/gb = U15977/ | 1072.4 | P | 1346.2 | P | 1032.5 | P | 802.5 |
| | | gi = 563830/ug = Mm 28962/len = 2100/STRA = for | | | | | | | |
| 5 | 95077_at | Cluster Incl U12922 RAB geramylgeranyl transferase, b subunit/cds = (0,1019)/gb = U12922/gi = 1345081/ug = Mm 29211/ | 494.5 | P | 403.3 | P | 360.9 | P | 428 |
| | | len = 1174/STRA = for | | | | | | | |
| 5 | 95096_at | Cluster Incl AI846695 UI-M-AN1-afi-f-06-0-UI s1 Mus musculus cDNA, 3 end/clone = UI-M-AN1-afi-f-06-0-UI/clone_end = 3/ | 1106.4 | P | 1181.3 | P | 933.2 | P | 915.1 |
| | | gb = AI846695/gi = 5490601/ug = Mm.29313/len = 374/STRA = for | | | | | | | |
| 5 | 95101_at | Cluster Incl AI834772: UI-M-AM0-ads-b-10-0-UI s1 Mus musculus cDNA, 3 end/clone = UI-M-AM0-ads-b-10-0-UI/ | 413.9 | P | 466 | P | 390.3 | P | 323.8 |
| | | clone_end = 3/gb = AI834772/gi = 5469005/ug = Mm 29344/len = 401/STRA = for | | | | | | | |
| 5 | 95647_f_at | Cluster Incl AI485345: vw50c09 y1 Mus musculus cDNA, 5 end/clone = IMAGE-1247248/clone_end = 5/gb = AI465845 | 857 | P | 920.8 | P | 585.7 | P | 662.6 |
| | | gi = 4319875/ug = Mm 29500/len = 327/STRA = for | | | | | | | |
| 5 | 95648_at | Cluster Incl AA655507: vs25d01.r1 Mus musculus cDNA, 5 end/clone = IMAGE-1139233/clone_end = 5/gb = AA655507/ | 761.8 | P | 774.3 | P | 558.9 | P | 640.6 |
| | | gi = 2591661/ug = Mm.29500/len = 522/STRA = for | | | | | | | |
| 5 | 95675_at | Cluster Incl AW121182 UI-M-BH2 3-aok-a-10-0-UI s1 Mus musculus cDNA, 3 end/cone = UI-M-BH2 3-aok-a-10-0-UI/ | 246.5 | P | 257.7 | P | 240.9 | P | 226.2 |
| | | clone_end = 3/gb = AW121182/gi = 6096515/ug = Mm 29588/len = 516/STRA = for | | | | | | | |
| 5 | 96256_at | Cluster Incl M28723 Anti-oxidant protein 1/cds = (47,820)/gb = M28723/gi = 309435/ug = Mm 29821/len = 1382/STRA = for | 489.8 | P | 335.6 | P | 412.1 | P | 324.2 |
| 5 | 96268_at | Cluster Incl AI840979 UI-M-AK0-adk-e-11-0-UI s1 Mus musculus cDNA,3 end/clone = UI-M-AK0-adk-e-11-0-UI/ | 792.6 | P | 1140 | P | 935.8 | P | 1022.8 |
| | | clone_end = 3/gb = AI840979/gi = 5475192/ug = Mm.29845/len = 405/STRA = for | | | | | | | |
| 5 | 96862_at | Cluster Incl AI848584 UI-M-AM1-aga-d-04-0-UI s2 Mus musculus cDNA, 3 end/clone = UI-M-AM1-aga-d-04-0-UI/ | 1594.9 | P | 1558.5 | P | 1127.4 | P | 1274.5 |
| | | clone_end = 3/gb = AI848584/gi = 5492490/ug = Mm 30053/len = 305/STRA = for | | | | | | | |
| 5 | 96871_at | Cluster Incl AI844355 UI-M-AL1-ahp-a-08-0-UI s1 Mus musculus cDNA, 3 end/clone = UI-M-AL1-ahp-a-08-0-UI/ | 585 | P | 391.9 | P | 462 | P | 426.1 |
| | | clone_end = 3/gb = AI844355/gi = 5488261/ug = Mm 30065/len = 330/STRA = for | | | | | | | |
| 5 | 96875_r_at | Cluster Incl AI841410: UI-M-AH0-acx-b-06-0-UI s1 Mus musculus cDNA, 3 end/clone = UI-M-AH0-acx-b-06-0-UI/ | 146 | P | 116.1 | P | 103.8 | P | 109.2 |
| | | clone_end = 3/gb = AI841410/gi = 5475623/ug = Mm 30069/len = 286/STRA = for | | | | | | | |
| 5 | 96881_at | Cluster Incl AW049394 UI-M-BH1-ane-e-07-0-UI s1 Mus musculus cDNA, 3 end/clone = UI-M-BH1-ane-e-07-0-UI/ | 470.4 | P | 448.2 | P | 389.1 | P | 412.7 |
| | | clone_end = 3/gb = AW049394/gi = 5909923/ug = Mm 30077/len = 543/STRA = for | | | | | | | |
| 5 | 96886_at | Cluster Incl AW060556 UI-M-BH1-ann-h-02-0-UI s1 Mus musculus cDNA, 3 end/clone = UI-M-BH1-ann-h-02-0-UI/ | 152.7 | P | 256.7 | P | 248.6 | P | 150.9 |
| | | clone_end = 3/gb = AW060556/gi = 6008307/ug = Mm.30083/len = 319/STRA = rev | | | | | | | |
| 5 | 96909_at | Cluster Incl AI849803 UI-M-AL1-aho-b-03-0-UI s1 Mus musculus cDNA, 3 end/clone = UI-M-AL1-aho-b-03-0-UI/ | 982 | P | 967.8 | P | 903.2 | P | 824.7 |
| | | clone_end = 3/gb = AI849803/gi = 5493709/ug = Mm.3014/len = 501/STRA = for | | | | | | | |
| 5 | 97448_at | Cluster Incl AI845165 UI-M-BG0-ahs-g-10-0-UI s1 Mus musculus cDNA, 3 end/clone = UI-M-BG0-ahs-g-10-0-UI/ | 663.2 | P | 501.7 | P | 417.4 | P | 563.6 |
| | | clone_end = 3/gb = AI845165/gi = 5489071/ug = Mm.30245/len = 214/STRA = for | | | | | | | |
| 5 | 97491_at | Cluster Incl AW123032 UI-M-BH2 1-apb-g-02-0-UI s1 Mus musculus cDNA, 3 end/clone = UI-M-BH2 1-apb-g-02-0-UI/ | 308.3 | P | 225.4 | P | 205.8 | P | 252.2 |
| | | clone_end = 3/gb = AW123032/gi = 6098562/ug = Mm 31051/len = 361/STRA = for | | | | | | | |
| 5 | 97502_at | Cluster Incl U73445: Dihydrolipoamide dehydrogenase/cds = (109,1638)/gb = U73445/gi = 2078521/ug = Mm 3131/len = 2163/ | 1585.5 | P | 1671.9 | P | 1092.4 | P | 1536.3 |
| | | STRA = for | | | | | | | |
| 5 | 98065_at | Cluster Incl AI837679 UI-M-AK0-adj-b-10-0-UI.s1 Mus musculus cDNA, 3 end/clone = UI-M-AK0-adj-b-10-0-UI/ | 831 | P | 865.8 | P | 767.7 | P | 889.6 |
| | | clone_end = 3/gb = AI837679/gi = 5471892/ug = Mm 34416/len = 159/STRA = for | | | | | | | |
| 5 | 98087_at | Cluster Incl AW048562 UI-M-BH1-alx-e-05-0-UI s1 Mus musculus cDNA, 3 end/clone = UI-M-BH1-alx-e-05-0-UI/ | 237.2 | P | 231.5 | P | 163.6 | P | 264.7 |
| | | clone_end = 3/gb = AW048562/gi = 5909091/ug = Mm 34580/len = 474/STRA = for | | | | | | | |
| 5 | 98121_at | Cluster Incl D49744 Farnesyltransferase, CAAX box, alpha/cds = (15,1148)/gb = D49744/gi = 1136744/ug = Mm.3496/ | 571.1 | P | 673.1 | P | 611.5 | P | 521.9 |
| | | len = 1629/STRA = for | | | | | | | |
| 5 | 98127_at | Cluster Incl U16741 Capping protein alpha 2/cds = (105,965)/gb = U16741/gi = 595918/ug = Mm.3529/len = 1690/STRA = for | 481.7 | P | 445.6 | P | 437.2 | P | 464.4 |
| 5 | 98524_f_at | Cluster Incl AI848479: UI-M-AP1-agf-a-10-0-UI.s2 Mus musculus cDNA, 3 end/clone = UI-M-AP1-agf-a-10-0-UI/ | 537.9 | P | 247.1 | P | 270 | P | 354.6 |
| | | clone_end = 3/gb = AI848479/gi = 5492385/ug = Mm 35823/len = 420/STRA = for | | | | | | | |
| 5 | 98531_g_at | Cluster Incl AI849615: UI-M-AH1-agr-g-09-0-UI s1 Mus musculus cDNA, 3 end/clone = UI-M-AH1-agr-g-09-0-UI/ | 1008.8 | P | 886.1 | P | 889.1 | P | 814.3 |
| | | clone_end = 3/gb = AI849615/gi = 5493521/ug = Mm 35844/len = 337/STRA = for | | | | | | | |
| 5 | 98540_g_at | Cluster Incl AF071312 Mus musculus COP9 complex subunit 2 (COPS2) mRNA, complete cds/cds = (12,1334)/ | 225.3 | P | 166.9 | P | 119.9 | P | 120.4 |
| | | gb = AF071312/gi = 3309165/ug = Mm.3596/len = 1929/STRA = for | | | | | | | |
| 5 | 98553_at | Cluster Incl AW124175: UI-M-BH2 1-apr-h-11-0-UI s1 Mus musculus cDNA, 3 end/clone = UI-M-BH2 1-apr-h-11-0-UI/ | 279.8 | P | 230.4 | P | 184.4 | P | 228.3 |
| | | clone_end = 3/gb = AW124175/gi = 6099705/ug = Mm 36769/len = 208/STRA = for | | | | | | | |
| 5 | 98587_at | Cluster Incl X61449: M. musculus mRNA for clone DN38/cds = (0,1034)/gb = X61449/gi = 50721/ug = Mm 3797/len = 1322/ | 996.5 | P | 623.4 | P | 620 | P | 545 |
| | | STRA = for | | | | | | | |
| 5 | 98595_at | Cluster Incl U56773 Interleukin 1 receptor-associated kinase/cds = (15,2048)/gb = U56773/gi = 1480173/ug = Mm.38241/ | 375.4 | P | 373.1 | P | 328.6 | P | 283.4 |
| | | len = 2703/STRA = for | | | | | | | |
| 5 | 99095_at | Cluster Incl M63903: Max protein/cds = (160,642)/gb = M63903/gi = 199986/ug = Mm 3931/len = 1941/STRA = for | 244.7 | P | 247.4 | P | 223.8 | P | 255.4 |
| 5 | 99577_at | Cluster Incl M57647: Mouse mast cell growth factor (MGF) mRNA, complete cds/cds = (149,970)/gb = M57647/gi = 19951/ | 630.4 | P | 436.5 | P | 124.9 | P | 103.1 |
| | | ug = Mm 4235/len = 2062/STRA = for | | | | | | | |
| 5 | 99604_at | Cluster Incl AI848713: UI-M-AJ1-ahf-h-10-0-UI.s1 Mus musculus cDNA, 3 end/clone = UI-M-AJ1-ahf-a-10-0-UI/ | 695.4 | P | 823.2 | P | 730 | P | 807.7 |
| | | clone_end = 3/gb = AI848713/gi = 5492619/ug = Mm 42925/len = 437/STRA = for | | | | | | | |
| 5 | 99621_s_at | Cluster Incl AA690583: vu53c05.r1 Mus musculus cDNA, 5 end/clone = IMAGE-1195112/clone_end = 5/gb = AA690583/ | 291.8 | P | 241.2 | P | 228.3 | P | 194.3 |
| | | gi = 2691519/ug = Mm 43213/len = 424/STRA = for | | | | | | | |
| 5 | 99628_at | Cluster Incl AF020308 Mus musculus HRS gene, complete cds/cds = (0,812)/gb = AF020308/gi = 2435502/ug = Mm 43331/ | 403.5 | P | 287.6 | P | 683.6 | P | 361.1 |
| | | len = 813/STRA = for | | | | | | | |
| 5 | 99649_at | Cluster Incl U85414 Glutamate cysteine ligase (gamma-glutamylcysteine synthetase), catalytic/cds = (0,1913)/gb = U85414/ | 201.2 | P | 140 | P | 107.2 | P | 99.1 |
| | | gi = 1815761/ug = Mm 4368/len = 1914/STRA = for | | | | | | | |
| 5 | 100089_at | Cluster Incl M74227 Mouse cyclophilin C (cyp C) mRNA, complete cds/cds = (51,689)/gb = M74227/gi = 192898/ | 948.6 | P | 957.1 | P | 1070.1 | P | 630.2 |
| | | ug = Mm 4587/len = 1287/STRA = for | | | | | | | |
| 5 | 100534_at | Cluster Incl AI183109: ub91h12 r1 Mus musculus cDNA, 5 end/clone = IMAGE-1395911/clone_end = 5/gb = AI183109/ | 628.8 | P | 650.1 | P | 623.2 | P | 549.5 |
| | | gi = 3733747/ug = Mm 5248/len = 358/STRA = for | | | | | | | |
| 5 | 100543_s_at | Cluster Incl AA620184: vo63e07.r1 Mus musculus cDNA, 5 end/clone = IMAGE-1054596/clone_end = 5/gb = AA620184/ | 271.7 | P | 260.8 | P | 279.7 | P | 279.6 |
| | | gi = 2524060/ug = Mm 5400/len = 507/STRA = for | | | | | | | |
| 5 | 100584_at | Cluster Incl U72941 Mus musculus annexin IV mRNA, complete cds/cds = (90,1049)/gb = U72941/gi = 1778312/ug = Mm 619/ | 427.1 | P | 504.7 | P | 437.9 | P | 423.6 |
| | | len = 1961/STRA = for | | | | | | | |
| 5 | 100608_at | Cluster Incl X95641: M. musculus mRNA for serine palmitoyltransferase subunit A/cds = (11,1432)/gb = X95641/gi = 2564281/ | 1115.1 | P | 1183.6 | P | 1288.7 | P | 1086.5 |
| | | ug = Mm 6505/len = 2576/STRA = for | | | | | | | |
| 5 | 101484_at | Cluster Incl U73039 Next to the Brca1/cds = (290,3256)/gb = U73039/gi = 1815651/ug = Mm.764/len = 4444/STRA = for | 310.7 | P | 279.3 | P | 372.1 | P | 316.3 |
| 5 | 101584_at | Cluster Incl X636039 M musculus RSP-1 mRNA for p33 protein/cds = (34,867)/gb = X63039/gi = 54014/ug = Mm.905/ | 468.8 | P | 378.3 | P | 472.7 | P | 469.3 |
| | | len = 1327/STRA = for | | | | | | | |
| 5 | 101590_at | Cluster Incl AI747194 ul13a12 x1 Mus musculus cDNA, 3 end/clone = IMAGE-2076670/clone_end = 3/gb = AI747194/ | 1049.3 | P | 1027.3 | P | 906.2 | P | 1161.3 |
| | | gi = 5125458/ug = Mm 91146/len = 585/STRA = for | | | | | | | |
| 5 | 101971_at | Cluster Incl AW061255: UI-M-BH1-amj-e-08-0-UI.s2 Mus musculus cDNA, 3 end/clone = UI-M-BH1-amj-e-08-0-UI/ | 355.5 | P | 307.5 | P | 269.5 | P | 170 |
| | | clone_end = 3/gb = AW061255/gi = 6009006/ug = Mm.9495/len = 495/STRA = for | | | | | | | |
| 5 | 102381_at | Cluster Incl AA619207: vo73e09.r1 Mus musculus cDNA, 5 end/clone = IMAGE-1064776/clone_end = 5/gb = AA619207/ | 209.9 | P | 174.3 | P | 116.9 | P | 137.9 |
| | | gi = 2523083/ug = Mm 12166/len = 367/STRA = for | | | | | | | |
| 5 | 102427_at | Cluster Incl AI849991 UI-M-BG0-aib-d-09-0-UI s1 Mus musculus cDNA, 3 end/clone = UI-M-BG0-aib-d-09-0-UI/ | 707.2 | P | 588.7 | P | 519.1 | P | 531.6 |
| | | clone_end = 3/gb = AI849991/gi = 5493897/ug = Mm.12831/len = 446/STRA = for | | | | | | | |
| 5 | 102781_at | Cluster Incl U37351 Mus musculus Paneth cell enhanced expression PCEE mRNA, complete cds/cds = (110,670)/ | 413.1 | P | 293.7 | P | 344.6 | P | 383.5 |
| | | gb = U37351/gi = 1276644/ug = Mm 13725/len = 1220/STRA = for | | | | | | | |
| 5 | 102850_at | Cluster Incl AF037260 Mus musculus non-receptor protein tyrosine kinase Ack mRNA, complete cds/cds = (267,3434)/ | 306.7 | P | 526.6 | P | 590.4 | P | 302.8 |
| | | gb = AF037260 gi = 2921446/ug = Mm 1483/len = 4214/STRA = for | | | | | | | |
| 5 | 102990_at | Cluster Incl AA655199: vv13b10.r1 Mus musculus cDNA, 5 end/clone = IMAGE-1211515/clone_end = 5/gb = AA655199/ | 1270.3 | P | 2061.1 | P | 1649.6 | P | 1083.5 |
| | | gi = 2591353/ug = Mm.1543/len = 367/STRA = for | | | | | | | |
| 5 | 103027_at | Cluster Incl AA739413 vv53b11 r1 Mus musculus cDNA, 5 end/clone = IMAGE-1226109/clone_end = 5/gb = AA739413/ | 254.6 | P | 269.3 | P | 257.3 | P | 225.8 |
| | | gi = 2775599/ug = Mm.15974/len = 388/STRA = for | | | | | | | |
| 5 | 103097_at | Cluster Incl AI844487: UI-M-AJ1-ahe-e-12-0-UI.s1 Mus musculus cDNA, 3 end/clone = UI-M-AJ1-ahe-e-12-0-UI/ | 184.3 | P | 182.1 | P | 195.5 | P | 192.6 |
| | | clone_end = 3/gb = AI844487/gi = 5488393/ug = Mm 17523/len = 363/STRA = for | | | | | | | |
| 5 | 103404_at | Cluster Incl AW122831: UI-M-BH2 1-aoz-d-08-0-UI s1 Mus musculus cDNA, 3 end/clone = UI-M-BH2 1-aoz-d-08-0-UI/ | 22.7 | P | 352.1 | P | 374.2 | P | 96.1 |
| | | clone_end = 3/gb = AW122831/gi = 6098361/ug = Mm 1878/len = 376/STRA = for | | | | | | | |
| 5 | 103574_at | Cluster Incl AI841606: UI-M-AP0-abn-h-07-0-UI s1 Mus musculus cDNA, 3 end/clone = UI-M-AP0-abn-h-07-0-UI/ | 601.9 | P | 522.3 | P | 641 | P | 450.2 |
| | | clone_end = 3/gb = AI841606/gi = 5475819/ug = Mm 1947/len = 378/STRA = for | | | | | | | |
| 5 | 103667_at | Cluster Incl AA866655 ud06h05 r1 Mus musculus cDNA, 5 end/clone = IMAGE-1434393/clone_end = 5/gb = AA866655/ | 299.7 | P | 252.3 | P | 165 | P | 195.3 |
| | | gi = 2962100/ug = Mm 20722/len = 542/STRA = for | | | | | | | |
| 5 | 103875_at | Cluster Incl AA967717: uh03f10.r1 Mus musculus cDNA, 5 end/clone = IMAGE-1616875/clone_end = 5/gb = AA967717/ | 165.8 | P | 174.2 | P | 109.5 | P | 93.8 |
| | | gi = 3141610/ug = Mm 21175/len = 433/STRA = for | | | | | | | |
| 5 | 103891_i_at | Cluster Incl AI197161: ue51h10.r1 Mus musculus cDNA, 5 end/clone = IMAGE-1494691/clone_end = 5/gb = AI197161/ | 966.5 | P | 940.1 | P | 737.3 | P | 1011.6 |
| | | gi = 3749767/ug = Mm 21288/len = 475/STRA = for | | | | | | | |
| 5 | 104056_at | Cluster Incl AI573367: ud42d02.y1 Mus musculus cDNA, 5 end/clone = IMAGE-1448547/clone_end = 5/gb = AI573367/ | 403.5 | P | 381.1 | P | 210.3 | P | 218.2 |
| | | gi = 4536741/ug = Mm 21532/len = 448/STRA = for | | | | | | | |
| 5 | 104063_at | Cluster Incl AI840130 UI-M-AO0-acc-a-09-0-UI s1 Mus musculus cDNA, 3 end/clone = UI-M-AO0-acc-a-09-0-UI/ | 212.2 | P | 181.6 | P | 173.2 | P | 152.4 |
| | | clone_end = 3/gb = AI840130/gi = 5474343/ug = Mm 21586/len = 421/STRA = for | | | | | | | |
| 5 | 104071_at | Cluster Incl AI852433 UI-M-BH0-ajo-g-04-0-UI.s1 Mus musculus cDNA, 3 end/clone = UI-M-BH0-ajo-g-04-0-UI/ | 727.1 | P | 825.7 | P | 766.5 | P | 802 |
| | | clone_end = 3/gb = AI852433/gi = 5496304/ug = Mm 21639/len = 544/STRA = for | | | | | | | |
| 5 | 104114_at | Cluster Incl AW122450 UI-M-BH2 2-aou-a-06-0-UI.s1 Mus musculus cDNA, 3 end/clone = UI-M-BH2.2-aou-a-06-0-UI/ | 273.7 | P | 259.6 | P | 201.1 | P | 209.8 |
| | | clone_end = 3/gb = AW122450/gi = 6097969/ug = Mm 21954/len = 288/STRA = for | | | | | | | |
| 5 | 104327_at | Cluster Incl AA755234 vr92d02 r1 Mus musculus cDNA, 5 end/clone = IMAGE-136163/clone_end = 5/gb = AA755234/ | 325 | P | 294.7 | P | 149 | P | 195.6 |
| | | gi = 2802432/ug = Mm 22459/len = 176/STRA = for | | | | | | | |
| 5 | 104332_at | Cluster Incl AI842242: UI-M-AI1-afp-d-07-0-UI s1 Mus musculus cDNA, 3 end/clone = UI-M-AI1-afp-d-07-0-UI/clone_end = 3/ | 527.5 | P | 473.4 | P | 368.5 | P | 456.2 |
| | | gb = AI842242/gi = 5476455/ug = Mm 22484/len = 349/STRA = for | | | | | | | |
| 5 | 104367_at | Cluster Incl AW124983 UI-M-BH2 1-apv-d-05-0-UI.s1 Mus musculus cDNA, 3 end/clone = UI-M-BH2 1-apv-d-05-0-UI/ | 478.9 | P | 371.7 | P | 285.3 | P | 275.5 |
| | | clone_end = 3/gb = AW124983/gi = 6100513/ug = Mm 22627/len = 398/STRA = for | | | | | | | |
| 5 | 104602_at | Cluster Incl AW121972 UI-M-BH2 3-aoj-a-09-0-UI s1 Mus musculus cDNA, 3 end/clone = UI-M-BH2 3-aoj-a-09-0-UI/ | 96.5 | P | 83 | P | 98.6 | P | 99.6 |
| | | clone_end = 3/gb = A121972/gi = 6097329/ug = Mm.24110/len = 474/STRA = for | | | | | | | |
| 5 | 104609_at | Cluster Incl AA644817: vs82f12 r1 Mus musculus cDNA, 5 end/clone = IMAGE-1152815/clone_end = 5/gb = AA644817/ | 443.9 | P | 452.2 | P | 440.8 | P | 361.1 |
| | | gi = 2571246/ug = Mm 24148/len = 382/STRA = for | | | | | | | |
| 5 | 104618_at | Cluster Incl AI845819 UI-M-AO1-aeg-b-07-0-UI.s1 Mus musculus cDNA, 3 end/clone = UI-M-AO1-aeg-b-07-0-UI/ | 188.2 | P | 168.4 | P | 167.3 | P | 173.9 |
| | | clone_end = 3/gb = AI845819/gi-5489725/ug = Mm 24216/len = 353/STRA = for | | | | | | | |
| 5 | 104621_at | Cluster Incl AW046060 UI-M-BH1-akv-d-12-0-UI s1 Mus musculus cDNA, 3 end/clone = UI-M-BH1-akv-d-12-0-UI/ | 899.5 | P | 872.8 | P | 641.2 | P | 649.7 |
| | | clone_end = 3/gb = AW046060/gi = 5906589/ug = Mm 24236/len = 322/STRA = for | | | | | | | |
| 5 | 104717_at | Cluster Incl AI848330 UI-M-AH1-ago-b-10-0-UI s1 Mus musculus cDNA, 3 end/clone = UI-M-AH1-ago-b-10-0-UI/ | 355.8 | P | 354.1 | P | 154.3 | P | 230.2 |
| | | clone_end = 3/gb = AI848330/gi = 5492236/ug = Mm 24505/len = 456/STRA = for | | | | | | | |
| 5 | 94399_at | Cluster Incl AI843172 UI-M-AK1-aet-b-11-0-UI s1 Mus musculus cDNA, 3 end/clone = UI-M-AK1-aet-b-11-0-UI/ | 175.5 | P | 313.3 | P | 160.4 | P | 169 |
| | | clone_end = 3/gb = AI843172/gi = 5477385/ug = Mm 25777/len = 383/STRA = for | | | | | | | |
| 5 | 94412_at | Cluster Incl AJ223733 Mus musculus CDK2 gene intron V containing the alternatively spliced exon Ve/cds = (0,123)/ | 572.6 | P | 583.3 | P | 455.1 | P | 293.9 |
| | | gb = AJ223733/gi = 2842420/ug = Mm 260/len = 124/STRA = for | | | | | | | |
| 5 | 94413_at | Cluster Incl AW21487: UI-M-BH2 2-aon-h-09-0-UI.s1 Mus musculus cDNA, 3 end/clone = UI-M-BH2.2-aon-h-09-0-UI/ | 251.4 | P | 197.1 | P | 176.5 | P | 222.4 |
| | | clone_end = 3/gb = AW121487/gi = 6096820/ug = Mm 26089/len = 168/STRA = rev | | | | | | | |
| 5 | 94435_at | Cluster Incl AI839117: UI-M-AO0-ach-b-04-0-UI.s1 Mus musculus cDNA, 3 end/clone = UI-M-AO0-ach-b-04-0-UI/ | 201 | P | 164.2 | P | 68.5 | P | 131.6 |
| | | clone_end = 3/gb = AI839117/gi = 5473330/ug = Mm 26537/len = 441/STRA = for | | | | | | | |
| 5 | 94439_at | Cluster Incl AW124363 UI-M-BH2 1-apq-h-10-0-UI s1 Mus musculus cDNA, 3 end/clone = UI-M-BH2 1-apq-h-10-0-UI/ | 275.2 | P | 362.4 | P | 313.5 | P | 282.3 |
| | | clone_end = 3/gb = AW124363/gi = 6099893/ug = Mm 26564/len = 253/STRA = for | | | | | | | |
| 5 | 94975_at | Cluster Incl AI845607: UI-M-AQ1-adx-e-10-0-UI s1 Mus musculus cDNA, 3 end/clone = UI-M-AQ1-adx-e-10-0-UI/ | 221.1 | P | 200.6 | P | 81.5 | P | 145.4 |
| | | clone_end = 3/gb = AI845607/gi = 5489513/ug = Mm 27243/len = 370/STRA = for | | | | | | | |
| 5 | 95021_at | Cluster Incl AW124028: UI-M-BH2.1-apn-c-06-0-UI s1 Mus musculus cDNA, 3 end/clone = UI-M-BH2 1-apn-c-06-0-UI/ | 535 | P | 449.6 | P | 405.5 | P | 469.6 |
| | | clone_end = 3/gb = AW124028/gi = 6099558/ug = Mm 27476/len = 440/STRA = for | | | | | | | |
| 5 | 96130_at | Cluster Incl AI822531: vw13g10 r1 Mus musculus cDNA, 5 end/clone = IMAGE-1243746/clone_end = 5/gb = AA822531/ | 494.4 | P | 695.9 | P | 517.4 | P | 531.5 |
| | | gi = 2892399/ug = Mm 28133/len = 426/STRA = for | | | | | | | |
| 5 | 96191_at | Cluster Incl AI852124: UI-M-BH0-ajb-c-05-0-UI s1 Mus musculus cDNA, 3 end/clone = UI-M-BH0-ajb-c-05-0-UI/ | 547.7 | P | 502.7 | P | 382 | P | 445.9 |
| | | clone_end = 3/gb = AI852124/gi = 546030/ug = Mm 28512/len = 275/STRA = for | | | | | | | |
| 5 | 96192_at | Cluster Incl AF062567: Mus musculus transcription factor Sp3 mRNA, partial cds/cds = (0,2177)/gb = AF062567/gi = 3132609/ | 608.5 | P | 536.2 | P | 291.2 | P | 435.6 |
| | | ug = Mm 28513/len = 3616/STRA = for | | | | | | | |
| 5 | 96199_at | Cluster Incl AF073996 Mus musculus myotubularin (Mtm1) mRNA, complete cds/cds = (75,1886)/gb = AF073996/ | 130.8 | P | 100.9 | P | 78.8 | P | 98.6 |
| | | gi = 3882999/ug = Mm.28580/len = 3339/STRA = for | | | | | | | |
| 5 | 96238_at | Cluster Incl AI853996: UI-M-BH0-aiv-g-11-0-UI.s1 Mus musculus cDNA, 3 end/clone = UI-M-BH0-aiv-g-11-0-UI/ | 151.1 | P | 118.6 | P | 194.7 | P | 155.3 |
| | | clone_end = 3/gb = AI853996/gi = 5497902/ug = Mm.28807/len = 255/STRA = for | | | | | | | |
| 5 | 96726_at | Cluster Incl AW049619: UI-M-BH1-and-d-10-0-UI.s1 Mus musculus cDNA, 3 end/clone = UI-M-BH1-and-d-10-0-UI/ | 314.4 | P | 299.7 | P | 250.7 | P | 264.8 |
| | | clone_end = 3/gb = AW049619/gi = 5910148/ug = Mm.28837/len = 423/STRA = for | | | | | | | |
| 5 | 96751_at | Cluster Incl AI842674 UI-M-AO1-aeo-a-12-0-UI.s1 Mus musculus cDNA, 3 end/clone = UI-M-AO1-aeo-a-12-0-UI/ | 1255.9 | P | 1305 | P | 1039.4 | P | 1002.5 |
| | | clone_end = 3/gb = AI842674/gi = 5476887/ug = Mm.28972/len = 341/STRA = for | | | | | | | |
| 5 | 96762_at | Cluster Incl AW046160 UI-M-BH1-akw-b-02-0-UI s1 Mus musculus cDNA, 3 end/clone = UI-M-BH1-akw-b-02-0-UI/ | 787.7 | P | 723 | P | 614.5 | P | 654.1 |
| | | clone_end = 3/gb = AW046160/gi = 5906689/ug = Mm 29005/len = 532/STRA = rev | | | | | | | |
| 5 | 96777_at | Cluster Incl AW049372: UI-M-BH1-ane-b-10-0-UI.s1 Mus musculus cDNA, 3 end/clone = UI-M-BH1-ane-b-10-0-UI/ | 300 | P | 333.9 | P | 267.8 | P | 298.2 |
| | | clone_end = 3/gb = AW049372/gi = 5909901/ug = Mm 29056/len = 322/STRA = for | | | | | | | |
| 5 | 97948_at | Cluster Incl M26391 Retinoblastoma 1/cds = (98,2863)/gb = M26391/gi = 200452/ug = Mm.304/len = 4591/STRA = for | 127.1 | P | 155.1 | P | 108.6 | P | 130.5 |
| 5 | 98455_at | Cluster Incl U46026 Activating transcription factor 2/cds = (0,731)/gb = U46026/gi = 1655802/ug = Mm 3466/len = 969/ | 214.2 | P | 111.6 | P | 84.1 | P | 78.6 |
| | | STRA = for | | | | | | | |
| 5 | 98503_at | Cluster Incl U53586 Ecotropic viral integration site 5/cds = (68,2497)/gb = U53586/gi = 1777918/ug = Mm 35796/len = 5870/ | 242 | P | 294.9 | P | 173.3 | P | 224 |
| | | STRA = for | | | | | | | |
| 5 | 98981_s_at | Cluster Incl X64840: Transcription factor 12/cds = (66,2186)/gb = X64840/gi = 49924/ug = Mm 36894/len = 3007/STRA = for | 671.8 | P | 706.5 | P | 576.9 | P | 584.5 |
| 5 | 99013_f_at | Cluster Incl AI846797: UI-M-AO1-ael-h-03-0-UI.s1 Mus musculus cDNA, 3 end/clone = UI-M-AO1-ael-h-03-0-UI/ | 2279.1 | P | 2420.7 | P | 2050.5 | P | 2106.8 |
| | | clone_end = 3/gb = AI846797/gi = 5490703/ug = Mm 38445/len = 383/STRA = for | | | | | | | |
| 5 | 99070_at | Cluster Incl U12473 Conserved helix-loop-helix ubiquitous kinase/cds = (53,2290)/gb = U12473/gi = 1079492/ug = Mm 3996/ | 183.7 | P | 131.3 | P | 157.3 | P | 217.3 |
| | | len = 3466/STRA = for | | | | | | | |
| 5 | 99491_at | Cluster Incl U53696 interleukin 10 receptor, beta/cds = (2,1051)/gb = U53696/gi = 1305488/ug = Mm.4154/len = 1670/ | 491 | P | 567.8 | P | 582.3 | P | 656.9 |
| | | STRA = for | | | | | | | |
| 5 | 99493_at | Cluster Incl AI842066 UI-M-AN1-afg-a-11-0-UI s1 Mus musculus cDNA, 3 end/clone = UI-M-AN1-afg-a-11-0-UI/ | 116.1 | P | 210.4 | P | 120.8 | P | 98.1 |
| | | clone_end = 3/gb = AI842066/gi = 5476279/ug = Mm 41555/len = 302/STRA = for | | | | | | | |
| 5 | 99513_at | Cluster Incl M72394 Mouse calcium-dependent phospholipid binding protein (PLA2) mRNA, complete cds/cds = (108,2354)/ | 196.6 | P | 167.5 | P | 114 | P | 121.1 |
| | | gb = M72394/gi = 200390/ug = Mm.4186/len = 2787/STRA = for | | | | | | | |
| 5 | 99552_at | Cluster Incl U79550.Slug, chicken homolog/cds = (43,852)/gb = U79550/gi = 1711202/ug = Mm 4272/len = 1945/STRA = for | 920.1 | P | 888.3 | P | 452.5 | P | 510.1 |
| 5 | 99955_at | Cluster Incl U70674: Mus musculus m-Numb (m-nb) mRNA, complete cds/cds = (166,1947)/gb = U70674/gi = 1575755/ | 208.3 | P | 189.9 | P | 157.3 | P | 148.6 |
| | | ug = Mm 4390/len = 3331/STRA = for | | | | | | | |
| 5 | 99964_at | Cluster Incl AW061016 UI-M-BH1-amn-f-06-0-UI s1 Mus musculus cDNA, 3 end/clone = UI-M-BH1-amn-f-06-0-UI/ | 138.7 | P | 99.3 | P | 80.7 | P | 63.2 |
| | | clone_end = 3/gb = AW061016/gi = 6008767/ug = Mm 44170/len = 435/STRA = for | | | | | | | |
| 5 | 99994_at | Cluster Incl AF041376: Mus musculus cell death activator CIDE-A (Cide-a) mRNA, complete cds/cds = (213,868)/ | 451.1 | P | 238.9 | P | 226.5 | P | 424.6 |
| | | gb = AF041376/gi = 3114591/ug = Mm 449/len = 1114/STRA = for | | | | | | | |
| 5 | 100019_at | Cluster Incl D45889 Chondroitin sulfate proteoglycan 2/cds = (0,124)/gb = D45889/gi = 994777/ug = Mm.4575/len = 2201/ | 509.4 | P | 619.3 | P | 290.8 | P | 332.6 |
| | | STRA = for | | | | | | | |
| 5 | 100878_at | Cluster Incl AW124985: UI-M-BH2.1-apv-d-07-0-UI.s1 Mus musculus cDNA, 3 end/clone = UI-M-BH2-1-apv-d-07-0-UI/ | 369.1 | P | 364.4 | P | 220.1 | P | 275.4 |
| | | clone_end = 3/gb = AW124985/gi = 6100515/ug = Mm 531/len = 339/STRA = for | | | | | | | |
| 5 | 100905_at | Cluster Incl AI849361 UI-M-AJ1-aha-g-09-0-UI s1 Mus musculus cDNA, 3 end/clone = UI-M-AJ1-aha-g-09-0-UI/ | 172.5 | P | 210.8 | P | 199.9 | P | 161.8 |
| | | clone_end = 3/gb = AI849361/gi = 5493267/ug = Mm 5772/len = 132/STRA = for | | | | | | | |
| 5 | 100951_at | Cluster Incl AF014010.Polycystic kidney disease 2/cds = (83,2983)/gb = AF014010/gi = 2558834/ug = Mm 6442/len = 5134/ | 145.1 | P | 180.8 | P | 138.6 | P | 120.7 |
| | | STRA = rev | | | | | | | |
| 5 | 100957_at | Cluster Incl AA881160 vz07b02 r1 Mus musculus cDNA, 5 end/clone = IMAGE-1314987/clone_end = 5/gb = AA881160/ | 297 | P | 248.5 | P | 213 | P | 208.8 |
| | | gi = 2990470/ug = Mm 6509/len = 575/STRA = for | | | | | | | |
| 5 | 100984_at | Cluster Incl M63725 Mouse binding protein for T-cell receptor (TCR-ATF1) mRNA, complete cds/cds = (20,829)/ | 305.2 | P | 365.2 | P | 274.3 | P | 327.9 |
| | | gb = M63725/gi = 201821/ug = Mm.676/len = 2122/STRA = for | | | | | | | |
| 5 | 101906_at | Cluster Incl AA032310: MTA.G11.085 A Mus musculus cDNA, 5 end/clone = MTA.G11.085/clone_end = 5/gb = AA032310/ | 734 | P | 567.9 | P | 247.5 | P | 264.6 |
| | | gi = 1504156/ug = Mm.89817/len = 380/STRA = for | | | | | | | |
| 5 | 101914_at | Cluster Incl AI846484 UI-M-AN1-aff-a-11-0-UI.s1 Mus musculus cDNA, 3 end/clone = UI-M-AN1-aff-a-11-0-UI/ | 231.7 | P | 239 | P | 178.6 | P | 193.2 |
| | | clone_end = 3/gb = AI846484/gi = 5490390/ug = Mm.90698/len = 393/STRA = for | | | | | | | |
| 5 | 102302_at | Cluster Incl L16992 Mus musculus branched-chain alpha-ketoacid dehydrogenase E1 beta-subunit mRNA sequence/ | 331.5 | P | 363.7 | P | 243.4 | P | 272.5 |
| | | cds = UNKNOWN/gb = L16992/gi = 293303/ug = Mm 12819/len = 1390/STRA = for | | | | | | | |
| 5 | 102309_at | Cluster Incl AB012725.Mus musculus ZAN75 mRNA for zinc finger protein, complete cds/cds = (81,1823)/gb = AB012725/ | 116.6 | P | 113.9 | P | 83.4 | P | 72.4 |
| | | gi = 3298471/ug = Mm 12891/len = 2627/STRA = for | | | | | | | |
| 5 | 102644_at | Cluster Incl U13371-Mus musculus (C57BL/10 X C3H)F2 clone 1 5 novel mRNA from renin-expressing kidney tumor cell | 269.8 | P | 270.5 | P | 271.1 | P | 206.9 |
| | | line, partial sequence/cds = UNKNOWN/gb = U13371/gi = 987223/ug = Mm 1314/len = 1217/STRA = for | | | | | | | |
| 5 | 102710_at | Cluster Incl AF020313: Mus musculus proline-rich protein 48 mRNA, partial cds/cds = (0,895)/gb = AF020313/gi = 2624971/ | 162.1 | P | 122.3 | P | 93.3 | P | 138.2 |
| | | ug = Mm 14255/len = 2223/STRA = for | | | | | | | |
| 5 | 103201_at | Cluster Incl M86377: Mouse esk kinase mRNA, complete cds/cds = (37,2607)/gb = M86377/gi = 193109/ug = Mm.1904/ | 164.8 | P | 125.6 | P | 68.7 | P | 101.4 |
| | | len = 2924/STRA = for | | | | | | | |
| 5 | 103312_f_at | Cluster Incl AW047929 UI-M-BH1-aln-d-03-0-UI.s1 Mus musculus cDNA, 3 end/clone = UI-M-BH1-aln-d-03-0-UI/ | 367 | P | 390.9 | P | 326.4 | P | 333.2 |
| | | clone_end = 3/gb = AW047929/gi = 5908458/ug = Mm.21228/len = 461/STRA = rev | | | | | | | |
| 5 | 103440_at | Cluster Incl AW226540: up08h09.x1 Mus musculus cDNA, 3 end/clone = IMAGE-2651489/clone_end = 3/gb = AW226540/ | 303.6 | P | 244.4 | P | 180.9 | P | 210.2 |
| | | gi = 6555836/ug = Mm 21424/len = 462/STRA = for | | | | | | | |
| 5 | 103695_f_at | Cluster Incl AW047329 UI-M-BH1-amh-h-04-0-UI s1 Mus musculus cDNA, 3 end/clone = UI-M-BH1-amh-h-04-0-UI/ | 585.3 | P | 553.4 | P | 438 | P | 403.4 |
| | | clone_end = 3/gb = AW047329/gi = 5907858/ug = Mm 23994/len = 438/STRA = for | | | | | | | |
| 5 | 103704_at | Cluster Incl AW047625: UI-M-BH1-akm-f-09-0-UI s1 Mus musculus cDNA, 3 end/clone = UI-M-BH1-akm-f-09-0-UI/ | 216.6 | P | 228.2 | P | 231.1 | P | 229.3 |
| | | clone_end = 3/gb = AW047625/gi = 5908154/ug = Mm.24075/len = 326/STRA = for | | | | | | | |
| 5 | 103739_at | Cluster Incl AW230977 uo70h08 y1 Mus musculus cDNA, 5 end/clone = IMAGE-2647935/clone_end = 5/gb = AW230977/ | 352.7 | P | 360.8 | P | 283.4 | P | 286.8 |
| | | gi = 6560343/ug = Mm.24411/len = 424/STRA = for | | | | | | | |
| 5 | 103753_at | Cluster Incl AI159572: vz77c07.r1 Mus musculus cDNA, 5 end/clone = IMAGE-1332492/clone_end = 5/gb = AI159572/ | 270.5 | P | 224.9 | P | 178.5 | P | 211.9 |
| | | gi = 3692754/ug = Mm.24527/len = 476/STRA = for | | | | | | | |
| 5 | 103932_at | Cluster Incl AA655311: vq85f02.s1 Mus musculus cDNA, 5 end/clone = IMAGE-1109115/clone_end = 5/gb = AA655311/ | 383.1 | P | 602.4 | P | 371.1 | P | 337 |
| | | gi = 2591465/ug = Mm 25148/len = 416/STRA = for | | | | | | | |
| 5 | 103947_at | Cluster Incl AW048038 UI-M-BH1-afv-c-02-0-UI s1 Mus musculus cDNA, 3 end/clone = UI-M-BH1-alv-c-02-0-UI/ | 239.7 | P | 160.3 | P | 21.4 | P | 200.2 |
| | | clone_end = 3/gb = AW048038/gi = 5908532/ug = Mm 25345/len = 269/STRA = for | | | | | | | |
| 5 | 103969_at | Cluster Incl AA981154 vx57h02 r1 Mus musculus cDNA, 5 end/clone = IMAGE-1279347/clone_end = 5/gb = AA981154/ | 398 | P | 337.8 | P | 206.5 | P | 234.9 |
| | | gi = 3159690/ug = Mm 25715/len = 218/STRA = for | | | | | | | |
| 5 | 104004_at | Cluster Incl AI931876: uI67b12.x1 Mus musculus cDNA, 3 end/clone = IMAGE-2135615/clone_end = 3/gb = AI931876/ | 389.5 | P | 329.3 | P | 163.5 | P | 243.9 |
| | | gi = 5668361/ug = Mm 26610/len = 529/STRA = for | | | | | | | |
| 5 | 104015_at | Cluster Incl AW047992: UI-M-BH1-alv-e-08-0-UI s1 Mus musculus cDNA, 3 end/clone = UI-M-BHI-alv-e-08-0-UI/ | 391.5 | P | 414.9 | P | 388.4 | P | 352.6 |
| | | clone_end = 3/gb = AW047992/gi = 5908561/ug = Mm 26833/len = 478/STRA = for | | | | | | | |
| 5 | 104412_at | Cluster Incl AI153412.uc53a01 r1 Mus musculus cDNA, 5 end/clone = IMAGE-1429320/clone_end = 5/gb = AI153412/ | 321 | P | 295.7 | P | 365.7 | P | 237.5 |
| | | gi = 3681881/ug = Mm 28859/len = 507/STRA = for | | | | | | | |
| 5 | 104640_f_at | Cluster Incl AI464596: mz66c12 y1 Mus musculus cDNA, 5 end/clone = IMAGE-718390/clone_end = 5/gb = AI464598/ | 321.9 | P | 226.1 | P | 231.2 | P | 211.9 |
| | | gi = 4318626/ug = Mm.31114/len = 339/STRA = for | | | | | | | |
| 5 | 92302_at | Cluster Incl Z11664 Son of sevenless homolog 2, (Drosophila)/cds = (0,3893)/gb = Z11664/gi = 54136/ug = Mm 3770/ | 112.3 | P | 112.9 | P | 151.1 | P | 124.6 |
| | | len = 5253/STRA = for | | | | | | | |
| 5 | 92502_at | Cluster Incl X95504 M musculus mRNA for zinc finger protein/cds = UNKNOWN/gb = X95504/gi = 2828233/ug = Mm 42200/ | 254 | P | 322.3 | P | 280.8 | P | 106 |
| | | len = 3732/STRA = for | | | | | | | |
| 5 | 92513_at | Cluster Incl AJ002636 Mus musculus mRNA for nuclear protein SA2/cds = (279,3767)/gb = AJ002638/gi = 2644956/ | 392.7 | P | 312.3 | P | 179.7 | P | 244.3 |
| | | ug = Mm 42238/len = 3871/STRA = for | | | | | | | |
| 5 | 92660_f_at | Cluster Incl X92665 M. musculus mRNA for ubiquitin-conjugating enzyme UbcM3/cds = (153,734)/gb = X92665/gi = 107238/ | 555.3 | P | 540.2 | P | 459.4 | P | 436.3 |
| | | ug = Mm.4429/len = 1328/STRA = for | | | | | | | |
| 5 | 93190_at | Cluster Incl AW209179 uo02h05.x1 Mus musculus cDNA, 3 end/clone = IMAGE-2582265/clone_end = 3/gb = AW209179/ | 253.3 | P | 318.5 | P | 247 | P | 244.4 |
| | | gi = 6515119/ug = Mm 5547/len = 428/STRA = for | | | | | | | |
| 5 | 93877_at | Cluster Incl AI834777 UI-M-AM0-ads-c-04-0-UI s1 Mus musculus cDNA, 3 end/clone = UI-M-AM0-ads-c-04-0-UI/ | 327.3 | P | 388.4 | P | 217.4 | P | 252.2 |
| | | clone_end = 3/gb = AI834777/gi = 5469010/ug = Mm 886/len = 279/STRA = for | | | | | | | |
| 5 | 94732_at | Cluster Incl X99251: Repetin/cds = (65,3457)/gb = X99251/gi = 1806131/ug = Mm 1417/len = 4178/STRA = for | 121.4 | P | 139.3 | P | 96.7 | P | 396.6 |
| 5 | 94768_at | Cluster Incl D37790 Mouse mRNA for beta-1,4-galactosyltransferase/cds = UNKNOWN/gb = D37790/gi = 517126/ | 1426.1 | P | 1509.2 | P | 1410.2 | P | 1154.4 |
| | | ug = Mm 164/len = 3298/STRA = rev | | | | | | | |
| 5 | 95395_at | Cluster Incl AI225869 uI22e07 y1 Mus musculus cDNA, 5 end/clone = IMAGE-1852068/clone_end = 5/gb = AI225869/ | 248.8 | P | 208.8 | P | 117.2 | P | 131.5 |
| | | gi = 3808922/ug = Mm 23834/len = 388/STRA = for | | | | | | | |
| 5 | 97689_at | Cluster Incl M26071: Coagulation factor III/cds = UNKNOWN/gb = M26071/gi = 201924/ug = Mm 3742/len = 1821/STRA = for | 398.7 | P | 402.6 | P | 437 | P | 298.9 |
| 5 | 95787_s_at | Cluster Incl X87685 M musculus sterol camer protein 2-pseudogene/cds = (0,478)/gb = X87685/gi = 854333/ug = Mm 87814/ | 508.2 | P | 493.9 | P | 396.3 | P | 364.9 |
| | | len = 479/STRA = for | | | | | | | |
| 5 | 92542_at | Cluster Incl AI849035 UI-M-AH1-agw-a-06-0-UI s1 Mus musculus cDNA, 3 end/clone = UI-M-AH1-agw-a-06-0-UI/ | 391.7 | P | 238.4 | P | 111.5 | A | 382.8 |
| | | clone_end = 3/gb = AI849035/gi = 5492941/ug = Mm.10009/len = 377/STRA = for | | | | | | | |
| 5 | 92579_at | Cluster Incl L00993 Sjogren syndrome antigen B/cds = (140,1387)/gb = L00993/gi = 198712/ug = Mm.10508/len = 1961/ | 119.6 | P | 51.7 | P | 59.1 | P | 47.7 |
| | | STRA = for | | | | | | | |
| 5 | 92648_at | Cluster Incl U19521: Mus musculus vesicle transport protein (munc.18c) mRNA, complete cds/cds = (159,1937)/gb = U19521/ | 228.5 | P | 166.2 | P | 124.3 | P | 137.5 |
| | | gi = 642027/ug = Mm 12155/len = 2037/STRA = for | | | | | | | |
| 5 | 93253_at | Cluster Incl D87271: Mus musculus DNA for ERK2/cds = (223,1098)/gb = D87271/gi = 2506074/ug = Mm 1700/len = 1182/ | 106.5 | P | 133.6 | P | 151.3 | P | 147.9 |
| | | STRA = for | | | | | | | |
| 5 | 95438_at | Cluster Incl AI846783 UI-M-AO1-ael-f-12-0-UI s1 Mus musculus cDNA, 3 end/clone = UI-M-AO1-ael-f-12-0-UI/ | 161.6 | P | 196.4 | P | 153.1 | P | 146.2 |
| | | clone_end = 3/gb = AI846783/gi = 5490689/ug = Mm 24541/len = 269/STRA = for | | | | | | | |
| 5 | 96616_at | Cluster Incl AI891629 ul57e11.x1 Mus musculus cDNA, 3 end/clone = IMAGE-2123180/clone_end = 3/gb = AI891629/ | 294.2 | P | 216.6 | P | 170.6 | P | 211.8 |
| | | gi = 5597531/ug = Mm 27123/len = 443/STRA = for | | | | | | | |
| 5 | 96266_at | Cluster Incl AI841982 UI-M-AHO-acu-h-04-0-UI s2 Mus musculus cDNA, 3 end/clone = UI-M-AH0-acu-h-04-0-UI/ | 831.5 | P | 581.4 | P | 486.3 | P | 420.6 |
| | | clone_end = 3/gb = AI841982/gi = 5476195/ug = Mm 29841/len = 334/STRA = for | | | | | | | |
| 5 | 99194_at | Cluster Incl AW124271 UI-M-BH2 1-aph-h-11-0-UI s1 Mus musculus cDNA, 3 end/clone = UI-M-BH2.1-aph-h-11-0-UI/ | 101.9 | P | 93.5 | P | 71.2 | P | 104.6 |
| | | clone_end = 3/gb = AW124271/gi = 6099801/ug = Mm 41935/len = 363/STRA = for | | | | | | | |
| 5 | 101475_at | Cluster Incl M64068 Mouse zinc finger protein (bmi-1) gene, complete cds/cds = (0,749)/gb = M64068/gi = 192204/ | 422.6 | P | 259.3 | P | 136.5 | P | 142.9 |
| | | ug = Mm.7719/len = 750/STRA = for | | | | | | | |
| 5 | 101965_at | Cluster Incl AF037205 Mus musculus RING zinc finger protein (Rzf) mRNA, complete cds/cds = (88,1233)/gb = AF037205/ | 309.3 | P | 279.1 | P | 252.8 | P | 225.6 |
| | | gi = 2746334/ug = Mm.9326/len = 2240/STRA = for | | | | | | | |
| 5 | 103083_at | Cluster Incl U69543 Lipase, hormone sensitive/cds = (592,2871)/gb = U69543/gi = 1899053/ug = Mm 1721/len = 3175/ | 470.4 | P | 499.3 | P | 391.8 | P | 410.4 |
| | | STRA = for | | | | | | | |
| 5 | 103328_at | Cluster Incl U59864 Mus musculus TRAF-interacting protein I-TRAF mRNA, complete cds/cds = (68,1414)/gb = U59864/ | 168.5 | P | 92.1 | P | 85.1 | P | 133 |
| | | gi = 1518019/ug = Mm 1803/len = 1971/STRA = for | | | | | | | |
| 5 | 103596_at | Cluster Incl AF085219: Mus musculus alpha diacylglycerol kinase mRNA, complete cds/cds = (19,2211)/gb = AF085219/ | 125 | P | 97 | P | 121.8 | P | 105.2 |
| | | gi = 3493665/ug = Mm 18981/len = 2239/STRA = for | | | | | | | |
| 5 | 103638_at | Cluster Incl AW046460 UI-M-BH1-akp-g-06-0-UI s1 Mus musculus cDNA, 3 end/clone = UI-M-BH1-akp-g-06-0-UI/ | 72.5 | P | 72.1 | P | 75.4 | P | 60 |
| | | clone_end = 3/gb = AW046460/gi = 5906989/ug = Mm.2035/len = 278/STRA = for | | | | | | | |
| 5 | 104060_at | Cluster Incl AI845977 UI-M-AP1-agi-b-12-0-UI s1 Mus musculus cDNA, 3 end/clone = UI-M-AP1-agl-b-12-0-UI/ | 217.1 | P | 219.8 | P | 207.1 | P | 176.7 |
| | | clone_end = 3/gb = AI845977/gi = 5489883/ug = Mm 21568/len = 351/STRA = for | | | | | | | |
| 5 | 104070_at | Cluster Incl AW047728 UI-M-BH1-alk-a-01-0-UI s1 Mus musculus cDNA, 3 end/clone = UI-M-BH1-alk-a-01-0-UI/ | 220.8 | P | 184.2 | P | 125.1 | P | 166.8 |
| | | clone_end = 3/gb = AW047728/gi = 5908257/ug = Mm 21637/len = 392/STRA = for | | | | | | | |
| 5 | 104290_at | Cluster Incl AI847837 UI-M-AM1-afu-b-08-o-UI.s1 Mus musculus cDNA, 3 end/clone = UI-M-AM1-afu-b-08-0-UI/ | 186.8 | P | 135.5 | P | 60.3 | P | 126.3 |
| | | clone_end = 3/gb = AI847837/gi = 5491743/ug = Mm 22279/len = 387/STRA = rev | | | | | | | |
| 5 | 104558_at | Cluster Incl AA867123: vx23f05.r1 Mus musculus cDNA, 5 end/clone = IMAGE-1265313/clone_end = 5/gb = AA867123/ | 372.2 | P | 261.5 | P | 225 | P | 204 |
| | | gi = 2962568/ug = Mm.23808/len = 552/STRA = for | | | | | | | |
| 5 | 104706_at | Cluster Incl U69171: Mus musculus peroxisomal PTS2 receptor mRNA, complete cds/cds = (92,1048)/gb = U69171/ | 148.1 | P | 144.9 | P | 128.2 | P | 107.8 |
| | | gi = 1890656/ug = Mm 2440/len = 1468/STRA = for | | | | | | | |
| 5 | 94923_f_at | Cluster Incl AA543502 vk25c02 r1 Mus musculus cDNA, 5 end/clone = IMAGE-947618/clone_end = 5/gb = AA543502/ | 549.1 | P | 621.7 | P | 268.1 | P | 435 |
| | | gi = 2291979/ug = Mm 26609/len = 598/STRA = rev | | | | | | | |
| 5 | 94992_at | Cluster Incl AI840667 UI-M-AM0-adr-b-05-0-UI s1 Mus musculus cDNA, 3 end/clone = UI-M-AM0-adr-b-05-0-UI/ | 346.9 | P | 354.6 | P | 257.2 | P | 327.4 |
| | | clone_end = 3/gb = AI840667/gi = 5474875/ug = Mm 27327/len = 304/STRA = for | | | | | | | |
| 5 | 95040_at | Cluster Incl AI840810 UI-M-AH0-adb-b-09-0-UI s1 Mus musculus cDNA, 3 end/clone = UI-M-AH0-adb-b-09-0-UI/ | 136.9 | P | 74 | P | 148.4 | P | 98.8 |
| | | clone_end = 3/gb = AI840810/gi = 5475023/ug = Mm 27569/len = 347/STRA = for | | | | | | | |
| 5 | 96215_f_at | Cluster Incl AI153421 uc53a11.r1 Mus musculus cDNA, 5 end/clone = IMAGE-1429340/clone_end = 5/gb = AI153421/ | 379.5 | P | 371.3 | A | 371.5 | P | 647.2 |
| | | gi = 3681890/ug = Mm 28674/len = 385/STRA = for | | | | | | | |
| 5 | 97422_at | Cluster Incl AW048882 UI-M-BH1-amf-e-12-0-UI s1 Mus musculus cDNA, 3 end/clone = UI-M-BH1-amf-e-12-0-UI/ | 156.6 | P | 161.5 | P | 98.2 | P | 175.2 |
| | | clone_end = 3/gb = AW048882/gi = 5909411/ug = Mm 29999/len = 497/STRA = for | | | | | | | |
| 5 | 97976_at | Cluster Incl L43326.Kinectin 1/cds = (19,4002)/gb = L43326/gi = 887379/ug = Mm 3110/len = 4466/STRA = rev | 124.7 | P | 94.2 | P | 49.8 | P | 93.8 |
| 5 | 103727_at | Cluster Incl AI850972 UI-M-BH0-akd-g-07-0-UI s1 Mus musculus cDNA, 3 end/clone = UI-M-BH0-akd-g-07-0-UI/ | 116.3 | P | 109.6 | P | 118.4 | P | 50.8 |
| | | clone_end = 3/gb = AI850972/gi = 5494878/ug = Mm 24291/len = 370/STRA = for | | | | | | | |
| 5 | 104193_at | Cluster Incl AW047583 UI-M-BH1-akm-b-04-0-UI a1Mus musculus cDNA, 3 end/clone = UI-M-BH1-akm-b-04-0-UI/ | 216.4 | P | 182.6 | P | 116.6 | P | 156.2 |
| | | clone_end = 3/gb = AW047583/gi = 5908112/ug = Mm 27493/len = 426/STRA = for | | | | | | | |
| 5 | 104683_at | Cluster Incl AW120783 UI-M-BH2.3-any-g-06-0-UI.s1 Mus musculus cDNA, 3 end/clone = UI-M-BH2.3-any-g-06-0-UI/ | 176.9 | P | 175.1 | P | 153.9 | P | 136.9 |
| | | clone_end = 3/gb = AW120783/gi = 6096116/ug = Mm.3289/len = 422/STRA = for | | | | | | | |
| 5 | 93203_f_at | Cluster Incl AW107463 um17a12x1 Mus musculus cDNA, 3 end/clone = IMAGE-2192542/clone_end = 3/gb = AW107463/ | 89.2 | P | 104.8 | P | 91.8 | P | 78.8 |
| | | gi = 6078263/ug = Mm.5697/len = 484/STRA = rev | | | | | | | |
| 5 | 102130_f_at | Cluster Incl C88243 C88243 Mus musculus cDNA, 3 end/clone = J0259A06/clone_end = 3/gb = C88243/gi = 2920200/ | 188 | P | 205.8 | P | 94.9 | P | 205.6 |
| | | ug = Mm 43522/len = 610/STRA = rev | | | | | | | |
| 5 | 96608_at | Cluster Incl AF023463 Mus musculus peroxisomal phytanoyl-CoA alpha-hydroxylase (PAHX) mRNA, complete cds/ | 135.6 | P | 178.1 | P | 82 | P | 139.8 |
| | | cds = (39,1055)/gb = AF023463/gi = 2564672/ug = Mm 27066/len = 1419/STRA = for | | | | | | | |
| 5 | 97853_at | Cluster Incl AW047331.UI-M-BH1-amh-h-07-0-UI s1 Mus musculus cDNA, 3 end/clone = UI-M-BH1-amh-h-07-0-UI/ | 152.6 | P | 95.7 | P | 35.8 | A | 71.9 |
| | | clone_end = 3/gb = AW047331/gi = 5907860/ug = Mm 28287/len = 333/STRA = for | | | | | | | |
| 5 | 97869_at | Cluster Incl AI844043 UI-M-AL1-ahi-d-11-0-UI s1 Mus musculus cDNA, 3 end/clone = UI-M-AL1-ahi-d-11-0-UI/ | 136.5 | A | 182.4 | P | 180.9 | P | 182.4 |
| | | clone_end = 3/gb = AI844043/gi = 5487949/ug = Mm.28336/len = 378/STRA = for | | | | | | | |
| 5 | 97892_at | Cluster Incl AI844732 UI-M-AL1-ahq-c-04-0-UI s1 Mus musculus cDNA, 3 end/clone = UI-M-AL1-ahq-c-04-0-UI/ | 61.8 | P | 56.1 | P | 19.8 | M | 52.4 |
| | | clone_end = 3/gb = AI844732/gi = 5488638/ug = Mm 28414/len = 475/STRA = for | | | | | | | |
| 5 | 96850_at | Cluster Incl AI853842 UI-M-BH0-ajq-a-04-0-UI.s1 Mus musculus cDNA, 3 end/clone = UI-M-BH0-ajq-a-04-0-UI/ | 94.8 | P | 110.5 | P | 47.5 | A | 113.2 |
| | | clone_end = 3/gb = AI853642/gi = 5497546/ug = Mm 30036/len = 511/STRA = for | | | | | | | |
| 5 | 98071_f_at | Cluster Incl X77731 M. musculus mRNA for Deoxycytidine kinase/cds = (12,794)/gb = X7731/gi = 456676/ug = Mm 3446/ | 432.8 | P | 387 | P | 162.8 | P | 349.3 |
| | | len = 2812/STRA = for | | | | | | | |
| 5 | 101498_at | Cluster Incl AF042730 Mus musculus lithium-sensitive myo-inositol monophosphatase A1 (IMPA1) mRNA, complete cds/ | 82.7 | P | 125.4 | P | 89 | A | 99.7 |
| | | cds = (66,899)/gb = AF042730/gi = 2801800/ug = Mm 8089/len = 2167/STRA = for | | | | | | | |
| 5 | 101996_at | Cluster Incl M80739 1570 bp/cds = UNKNOWN/gb = M80739/gi = 485375/ug = Mm 985/len = 1570/STRA = for | 139.6 | P | 155.8 | P | 102.2 | P | 130 |
| 5 | 102318_at | Cluster Incl X86000 Mus musculus mRNA for N-glycan alpha 2,8-sialyltransferase/cds = (333,1412)/gb = X86000/ | 74.1 | P | 98.8 | P | 42 | P | 82.7 |
| | | gi = 2685331/ug = Mm 10701/len = 6786/STRA = for | | | | | | | |
| 5 | 102321_at | Cluster Incl M93422 Adenylate cyclase 6/cds = (95,3592)/gb = M93422/gi = M93422/gi = 191690/ug = Mm 10706/len = 5841/STRA = for | 232.7 | P | 70.4 | A | 243.3 | P | 86.4 |
| 5 | 104316_at | Cluster Incl AI646098: vw54h09 x1 Mus musculus cDNA, 3 end/clone = IMAGE-1247681/clone_end = 3/gb = AI646098/ | 128.5 | P | 185.8 | P | 152.6 | P | 160.6 |
| | | gi = 4724573/ug = Mm 22414/len = 501/STRA = rev | | | | | | | |
| 5 | 94397_at | Cluster Incl AA690091: vr79a09 s1 Mus musculus cDNA, 5 end/clone = IMAGE-1134904/clone_end = 5/gb = AA690091/ | 180.1 | P | 155.4 | A | 168 | P | 127.4 |
| | | gi = 2691027/ug = Mm 25776/len = 347/STRA = for | | | | | | | |
| 5 | 94979_at | Cluster Incl AW121624: UI-M-BH2 2-acq-g-11-0-UI.s1 Mus musculus cDNA, 3 end/clone = UI-M-BH2.2-aoq-g-11-0-UI/ | 80.2 | P | 54.6 | P | 16.6 | P | 15.9 |
| | | clone_end = 3/gb = AW121624/gi = 6096957/ug = Mm 27273/len = 334/STRA = for | | | | | | | |
| 5 | 95001_at | Cluster Incl AB028920 Mus musculus AKAP95 mRNA for A kinase anchor protein 95, complete cds/cds = (38,2101)/ | 140.2 | P | 126.5 | P | 144 | P | 136.4 |
| | | gb = AB028920/gi = 5931617/ug = Mm 27351/len = 2704/STRA = for | | | | | | | |
| 5 | 95020_at | Cluster Incl AI848868 UI-M-AJ1-ahb-f-03-0-UI s1 Mus musculus cDNA, 3 end/clone = UI-M-AJ1-ahb-f-03-0-UI/ | 197.3 | P | 200.1 | P | 227.8 | P | 161.5 |
| | | clone_end = 3/gb = AI848868/gi = 5492774/ug = Mm 27466/len = 496/STRA = for | | | | | | | |
| 5 | 95028_r_at | Cluster Incl C77009 C77009 Mus musculus cDNA, 3 end/clone = J0024D07/clone_end = 3/gb = C77009/gi = 2517339/ | 397.8 | P | 238.8 | P | 154.3 | A | 209 |
| | | ug = Mm 27511/len = 496/STRA = rev | | | | | | | |
| 5 | 97919_at | Cluster Incl AI837467 UI-M-AL0-abr-d-06-0-UI s1 Mus musculus cDNA,3 end/clone = UI-M-AL0-abr-d-06-0-UI/ | 167.9 | P | 112.1 | P | 51.8 | P | 71.7 |
| | | clone_end = 3/gb = AI837467/gi = 5471680/ug = Mm 30132/len = 228/STRA = for | | | | | | | |
| 5 | 98504_at | Cluster Incl U58513 Mus musculus Rho-associated, coiled-coil forming protein kinase p160 ROCK-2 mRNA, complete cds/ | 151.8 | P | 48.5 | P | 51.7 | P | 61.9 |
| | | cds = (0,4166)/gb = U58513/gi = 1514697/ug = Mm 35815/len = 4167/STRA = for | | | | | | | |
| 5 | 100894_at | Cluster Incl AJ007360 Mus musculus mRNA for ORC5-related protein/cds = (65,1372)/gb = AJ007360/gi = 4938333/ | 73.3 | P | 51.7 | P | 58.1 | P | 91.7 |
| | | ug = Mm 566/len = 2079/STRA = for | | | | | | | |
| 5 | 103202_at | Cluster Incl AW047476.UI-M-BH1-all-g-04-0-UI s1 Mus musculus cDNA, 3 end/clone = UI-M-BH1-all-g-04-0-UI/ | 177.8 | P | 199.4 | P | 163.9 | P | 336.7 |
| | | clone_end = 3/gb = AW047476/gi = 5908005/ug = Mm 1909/len = 248/STRA = for | | | | | | | |
| 5 | 103207_at | Cluster Incl D13543 DNA polymerase alpha 1, 180 kDa/cds = (21,4418)/gb = D13543/gi = 475158/ug = Mm.1923/len = 5323/ | 182.1 | P | 133.5 | P | 73.5 | P | 90.6 |
| | | STRA = for | | | | | | | |
| 5 | 92226_at | Cluster Incl AA866971 vx91g10.r1 Mus musculus cDNA, 5 end/clone = IMAGE-1282626/clone_end = 5/gb = AA866971/ | 255.3 | P | 294.1 | P | 204 | P | 116.6 |
| | | gi = 2962416/ug = Mm.346/len = 370/STRA = for | | | | | | | |
| 5 | 96526_at | Cluster Incl AW228840 up22f10.x1 Mus musculus cDNA, 3 end/clone = IMAGE-2655115/clone_end = 3/gb = AW228840/ | 138.4 | P | 143.9 | P | 112.4 | A | 156.9 |
| | | gi = 6558136/ug = Mm.2670/len = 133/STRA = for | | | | | | | |
| 5 | 98893_at | Cluster Incl AW048644 UI-M-BH1-amd-d-08-0-UI s1 Mus musculus cDNA, 3 end/clone = UI-M-BH1-amd-d-08-0-UI/ | 197.4 | A | 250.9 | P | 193.5 | P | 172.7 |
| | | clone_end = 3/gb = AW048644/gi = 5909173/ug = Mm 28550/len = 430/STRA = for | | | | | | | |
| 5 | 103010_at | Cluster Incl AW048174: UI-M-BH1-amc-b-12-0-UI.s1 Mus musculus cDNA, 3 end/clone = UI-M-BH1-amc-b-12-0-UI/ | 77 | A | 114.1 | A | 146.9 | P | 153.1 |
| | | clone _end = 3/gb = AW048174/gi = 5908703/ug = Mm.1571/len = 398/STRA = for | | | | | | | |
| 5 | 103076_at | Cluster Incl AW046093 UI-M-BH1-alf-a-06-0-UI s1 Mus musculus cDNA, 3 end/clone = UI-M-BH1-alf-a-06-0-UI/ | 455.2 | P | 398.9 | P | 328.5 | P | 352.7 |
| | | clone _end = 3/gb = AW046093/gi = 5906622/ug = Mm 17087/len = 258/STRA = for | | | | | | | |
| 5 | 103422_at | Cluster Incl M63695 CD1d1 antigen/cds = (91,1101)/gb = M63695/gi = 192471/ug = Mm 1894/len = 1838/STRA = for | 111.5 | P | 115 | P | 37.6 | P | 100.8 |
| 5 | 103895_at | Cluster Incl AW121745: UI-M-BH2 3-aoe-c-07-0-UI s1 Mus musculus cDNA, 3 end/clone = UI-M-BH2 3-aoe-c-07-0-UI/ | 202.9 | P | 131.2 | P | 26.5 | P | −1.5 |
| | | clone _end = 3/gb = AW121745/gi = 6097080/ug = Mm 21299/len = 345/STRA = for | | | | | | | |
| 5 | 104049_at | Cluster Incl AW047134: UI-M-BH1-alu-c-03-0-UI s1 Mus musculus cDNA, 3 end/clone = UI-M-BH1-alu-c-03-0-UI/ | 89.8 | P | 65.6 | P | 30.5 | A | 31.9 |
| | | clone _end = 3/gb = AW047134/gi = 5907663/ug = Mm 21510/len = 332/STRA = for | | | | | | | |
| 5 | 94934_at | Cluster Incl U04828: Angiotensin II receptor, type 2/cds = (131,1222)/gb = U04828/gi = 439862/ug = Mm.2679/len = 2827/ | 341.9 | P | 347.1 | P | 372.4 | P | 137.9 |
| | | STRA = for | | | | | | | |
| 5 | 96741_at | Cluster Incl AW124129: UI-M-BH2 1-apr-d-08-0-UI s1 Mus musculus cDNA, 3 end/clone = UI-M-BH2 1-apr-d-08-0-UI/ | 35 | P | 24.1 | A | −14.9 | A | 13.8 |
| | | clone _end = 3/gb = AW124129/gi = 6099659/ug = Mm 28932/len = 192/STRA = rev | | | | | | | |
| 5 | 99999_at | Cluster Incl D83033 Mouse mRNA for nuclear protein, NP220, complete cds/cds = (325,6105)/gb = D83033/gi = 1469808/ | 221 | P | 114.7 | P | 62.2 | A | 93 |
| | | ug = Mm 4503/len = 6345/STRA = for | | | | | | | |
| 5 | 100903_at | Cluster Incl AJ007780 Mus musculus mRNA for poly(ADP-ribose) polymerase-2/cds = (15,1694)/gb = AJ007780/ | 162.8 | P | 99.7 | P | 92.7 | P | 60.1 |
| | | gi = 4138232/ug = Mm 5728/len = 1707/STRA = for | | | | | | | |
| 5 | 104267_at | Cluster Incl AI844736: UI-M-AL1-ahq-c-10-0-UI s1 Mus musculus cDNA, 3 end/clone = UI-M-AL1-ahq-c-10-0-UI/ | 305.7 | P | 264.6 | A | 250.2 | M | 149.3 |
| | | clone _end = 3/gb = AI844736/gi = 5488642/ug = Mm 28535/len = 406/STRA = rev | | | | | | | |
| 5 | 93887_at | Cluster Incl AI854351 UI-M-BG1-aic-d-05-0-UI s1 Mus musculus cDNA, 3 end/clone = UI-M-BG1-aic-d-05-0-UI/ | 104.6 | P | 128.7 | P | 133.8 | P | 99.2 |
| | | clone _end = 3/gb = AI854351/gi = 5498257/ug = Mm 88860/len = 369/STRA = for | | | | | | | |
| 5 | 94282_at | Cluster Incl AW124297 UI-M-BH2 1-apq-c-02-0-UI s1 Mus musculus cDNA, 3 end/clone = UI-M-BH2 1-apq-c-02-0-UI/ | 219.5 | P | 179.6 | A | 255.3 | P | 200.2 |
| | | clone _end = 3/gb = AW124297/gi = 6099827/ug = Mm 22547/len = 237/STRA = for | | | | | | | |
| 5 | 96945_at | Cluster Incl U73143 Mus musculus syndet mRNA, complete cds/cds = (0,632)/gb = U73143/gi = 2065524/ug = Mm.30197/ | 75 | P | 70 | P | 85.1 | M | 78.6 |
| | | len = 633/STRA = for | | | | | | | |
| 5 | 102388_at | Cluster Incl AW121186: UI-M-BH2 3-aok-b-02-0-UI s1 Mus musculus cDNA, 3 end/clone = UI-M-BH2 3-aok-b-02-0-UI/ | 168 | P | 167.3 | A | 61.2 | M | 111.2 |
| | | clone _end = 3/gb = AW121186/gi = 6096519/ug = Mm 1182/len = 501/STRA = for | | | | | | | |
| 5 | 103813_at | Cluster Incl D85926: Mus musculus mRNA for Sh3yl1, complete cds/cds = (41,1063)/gb = D85926/gi = 1944388/ | 59.6 | P | 61.1 | A | 56 | P | 34.6 |
| | | ug = Mm 2090/len = 1226/STRA = for | | | | | | | |
| 5 | 104119_at | Cluster Incl AI845028: UI-M-BGO-ahy-a-01-0-UI s1 Mus musculus cDNA, 3 end/clone = UI-M-BGO-ahy-a-01-0-UI/ | 179.7 | P | 112.7 | A | 82.9 | P | 54.6 |
| | | clone_end = 3/gb = AI845028/gi = 5488934/ug = Mm.21977/len = 510/STRA = for | | | | | | | |
| 5 | 104380_at | Cluster Incl Z71268: M musculus mRNA for CMP-stalic acid transporter/cds = (0,1010)/gb = Z71268/gi = 1486407/ | 78.6 | P | 123.1 | M | 108.8 | P | 91 |
| | | ug = Mm 22684/len = 1185/STRA = for | | | | | | | |
| 5 | 104759_at | Cluster Incl AI851421: UI-M-BH0-akj-c-04-0-UI s1 Mus musculus cDNA, 3 end/clone = UI-M-BH0-akj-c-04-0-UI/ | 248.7 | A | 115.4 | P | 135.2 | P | 205.9 |
| | | clone_end = 3/gb = AI851421/gi = 5495327/ug = Mm 24797/len = 337/STRA = for | | | | | | | |
| 5 | 94386_at | Cluster Incl AI850140 UI-M-BG1-aid-c-11-0-UI s1 Mus musculus cDNA, 3 end/clone = UI-M-BG1-aid-c-11-0-UI/ | 237.6 | P | 248.1 | P | 291.8 | A | 230 |
| | | clone_end = 3/gb = AI850140/gi = 5494046/ug = Mm 25622/len = 305/STRA = for | | | | | | | |
| 5 | 95549_at | Cluster Incl D13545 DNA primase, p58 subunit/cds = (144,1661)/gb = D13545/gi = 303662/ug = Mm 27705/len = 1937/ | 115.9 | P | 89.1 | P | 67.5 | P | 47.2 |
| | | STRA = for | | | | | | | |
| 5 | 96746_at | Cluster Incl AW124813: UI-M-BH2.1-apk-c-01-0-UI.s1 Mus musculus cDNA, 3 end/clone = UI-M-BH2.1-apk-c-01-0-UI/ | 88.3 | P | 136.8 | A | 94 | A | 131 |
| | | clone_end = 3/gb = AW124813/gi = 6100343/ug = Mm 28953/len = 435/STRA = for | | | | | | | |
| 5 | 96785_at | Cluster Incl AF110520 Mus musculus major histocompatibility complex region NG27, NG28, RPS28, NADH | 129.4 | A | 157.1 | P | 146.8 | P | 97.8 |
| | | oxidoreductase NG29, KIFC1, Fas-binding protein, BING1, tapasin, RaIGDS-like, KE2, BING4, beta 1,3-galactosyl | | | | | | | |
| | | transferase, and RPS18 genes, complete cds, S/STRA = for | | | | | | | |
| 5 | 99441_at | Cluster Incl AW123852 UI-M-BH2 1-app-b-06-0-UI.s1 Mus musculus cDNA, 3 end/clone = UI-M-BH2.1-app-b-06-0-UI/ | 62.2 | P | 62.9 | P | 59.2 | P | 36.3 |
| | | clone_end = 3/gb = AW123852/gi = 6099382/ug = Mm 40282/len = 451/STRA = for | | | | | | | |
| 5 | 99963_at | Cluster Incl U07861: Zinc finger protein 101/cds = (107,1927)/gb = U07861/gi = 466390/ug = Mm 4417/len = 2177/STRA = rev | −7.3 | P | −25.9 | A | −32.6 | A | 14 |
| 5 | 100880_at | Cluster Incl AA816121 vp44a01.r1 Mus musculus cDNA, 5 end/clone = IMAGE-1079496/clone_end = 5/gb = AA816121/ | 168.2 | P | 105.1 | P | 46.2 | P | 193.3 |
| | | gi = 2885717/ug = Mm.532/len = 316/STRA = rev | | | | | | | |
| 5 | 102957_at | Cluster Incl U20159: Mus musculus 78 kDa tyrosine phosphoprotein SLP-76 mRNA, complete cds/cds = (192,1793)/ | 36.7 | P | 83.6 | P | 53.5 | M | 49.9 |
| | | gb = U20159/gi = 806767/ug = Mm 1781/len = 2247/STRA = for | | | | | | | |
| 5 | 103537_at | Cluster Incl AF076681: Mus musculus type-I transmembrane ER-resident serine/threonine kinase PERK mRNA, complete | 31.4 | P | 64.5 | P | 7.6 | A | 30.2 |
| | | cds/cds = (191,3535)/gb = AF076681/gi = 4107512/ug = Mm 23375/len = 4510/STRA = for | | | | | | | |
| 5 | 104670_at | Cluster Incl AI315698 uj34a01 x1 Mus musculus cDNA, 3 end/clone = IMAGE-1921800/clone_end = 3/gb = AI315698/ | 165.6 | P | 131.6 | P | 57.5 | A | 74 |
| | | gi = 4030965/ug = Mm 32357/len = 215/STRA = for | | | | | | | |
| 5 | 93241_r_at | Cluster Incl AI838094 UI-M-AL0-abv-g-07-0-UI s1 Mus musculus cDNA, 3 end/clone = UI-M-AL0-abv-g-07-0-UI/ | 61.1 | P | 45.7 | A | 38.3 | P | 54.6 |
| | | clone _end = 3/gb = AI838094/gi = 5472307/ug = Mm 5935/len = 344/STRA = rev | | | | | | | |
| 5 | 94088_at | Cluster Incl AW228429 up22e01 y1 Mus musculus cDNA, 5 end/clone = IMAGE-2655096/clone _end = 5/gb = AW228429/ | 105.7 | M | 85.9 | P | 101.5 | P | 59.1 |
| | | gi = 6557725/ug = Mm 99371/len = 342/STRA = for | | | | | | | |
| 5 | 95288_i_at | Cluster Incl AA189811 mt97g08.r1 Mus musculus cDNA, 5 end/clone = IMAGE-637886/clone_end = 5/gb = AA189811/ | 65.6 | P | 57.4 | P | 19.1 | A | 27.8 |
| | | gi = 1776416/ug = Mm 1882/len = 365/STRA = for | | | | | | | |
| 5 | 95939_i_at | Cluster Incl AW047237: UI-M-BH1-amb-g-05-0-UI.s1 Mus musculus cDNA, 3 end/clone = UI-M-BH1-amb-g-05-0-UI/ | 48.6 | P | 92 | P | 106.4 | A | 86.1 |
| | | clone_end = 3/gb = AW047237/gi = 5907766/ug = Mm 24851/len = 420/STRA = for | | | | | | | |
| 5 | 93538_at | Cluster Incl AW228036: Up12e07.x1 Mus musculus cDNA, 3 end/clone = IMAGE-2654148/clone_end = 3/gb = AW228036/ | 79.4 | P | 41.8 | P | 5.5 | A | 43.1 |
| | | gi = 6557332/ug = Mm 19992/len = 516/STRA = for | | | | | | | |
| 5 | 94812_at | Cluster Incl AA684175 vm68b10. s1 Mus musculus cDNA, 5 end/clone = IMAGE-1003387/clone_end = 5/gb = AA684175/ | 110.4 | P | 175.1 | A | 56.6 | A | 22.1 |
| | | gi = 2670761/ug = Mm 22700/len = 513/STRA = for | | | | | | | |
| 5 | 94818_at | Cluster Incl AW047223: UI-M-BH1-amb-e-09-0-UI.s1 Mus musculus cDNA, 3 end/clone = UI-M-BH1-amb-c-09-0-UI/ | 299.6 | P | 189.6 | P | 53.8 | A | 113.1 |
| | | clone_end = 3/gb = AW047223/gi = 5907752/ug = Mm 22710/len = 420/STRA = for | | | | | | | |
| 5 | 98048_at | Cluster Incl AF080490 Mus musculus TLS-associated protein TASR-2 mRNA, complete cds/cds = (91,879)/gb = AF060490/ | 206.7 | P | 175.2 | P | 22.7 | A | 89.2 |
| | | gi = 3327956/ug = Mm 34279/len = 2693/STRA = for | | | | | | | |
| 5 | 98543_at | Cluster Incl AJ223208: Mus musculus mRNA for cathepsin S, partial/cds = (0,139)/gb = AJ223208/gi = 4138249/ | 271.9 | P | 151 | P | 172.3 | P | 228.7 |
| | | ug = Mm.3619/len = 366/STRA = for | | | | | | | |
| 5 | 99178_at | Cluster Incl AI845652: UI-M-AQ1-ady-b-04-0-UI.a1 Mus musculus cDNA, 3 end/clone = UI-M-AQ1-ady-b-04-0-UI/ | 28.7 | P | 43.5 | P | 28.8 | A | 33.2 |
| | | clone_end = /gb = AI845652/gi = 5489558/ug = Mm.41791/len = 357/STRA = for | | | | | | | |
| 5 | 101082_at | Cluster Incl J02652: Malic enzyme, supernatant/cds = (65,1783)/gb = J02652/gi = 199762/ug = Mm 730/len = 3129/STRA = for | 310.1 | P | 403.8 | A | 336 | P | 205.9 |
| 5 | 94395_at | Cluster Incl AI194254 ul14a10.r1 Mus musculus cDNA, 5 end/clone = IMAGE-1851258/clone_end = 5/gb = AI194254/ | 95 | P | 33.1 | P | −8.8 | A | 16.8 |
| | | gi = 3745461/ug = Mm 25703/len = 475/STRA = for | | | | | | | |
| 5 | 96231_at | Cluster Incl AW123780 UI-M-BH2 1-apm-c-10-0-UI s1 Mus musculus cDNA, 3 end/clone = UI-M-BH2.1-apm-c-10-0-UI/ | 167.7 | A | 184.3 | P | 176.7 | P | −58.1 |
| | | clone_end = 3/gb = AW123780/gi = 6099310/ug = Mm 28775/len = 400/STRA = for | | | | | | | |
| 5 | 96778_at | Cluster Incl AA656775 vr50e03 s1 Mus musculus cDNA, 5 end/clone = IMAGE-1124092/clone_end = 5/gb = AA656775/ | 255.5 | P | 269.9 | P | 255.6 | A | 135 |
| | | gi = 2592929/ug = Mm 29061/len = 417/STRA = for | | | | | | | |
| 5 | 98026_g_at | Cluster Incl M34896: Ecotropic viral integration site 2/cds = (191,862)/gb = M34896/gi = 193202/ug = Mm.3266/len = 1217/ | 297.4 | P | 207.1 | A | 256.8 | P | 201.3 |
| | | STRA = for | | | | | | | |
| 5 | 98972_at | Cluster Incl AI574262 uj19e05 x1 Mus musculus cDNA, 3 end/clone = IMAGE-1920416/clone_end = 3/gb = AI574262/ | 0.2 | A | −132.7 | A | −82.4 | A | −98.8 |
| | | gi = 4537636/ug = Mm 36569/len = 474/STRA = for | | | | | | | |
| 5 | 104263_at | Cluster Incl AA914734 vy91g08.r1 Mus musculus cDNA, 5 end/clone = IMAGE-1313630/clone_end = 5/gb = AA914734/ | 66 | P | 38.5 | A | 39 | P | 19.4 |
| | | gi = 3054126/ug = Mm 28450/len = 470/STRA = rev | | | | | | | |
| 5 | 104274_at | Cluster Incl AW124843 UI-M-BH2.1.apk-f-10-0-UI s1 Mus musculus cDNA, 3 end/clone = UI-M-BH2 1-apk-f-10-0-UI/ | 275.9 | P | 413.9 | A | 228.5 | A | 416.2 |
| | | clone_end = 3/gb = AW124843/gi = 6100373/ug = Mm.28596/len = 509/STRA = for | | | | | | | |
| 5 | 104639_i_at | Cluster Incl AI464596 mz66c12.y1 Mus musculus cDNA, 5 end/clone = IMAGE-718390/clone_end = 5/gb = AI464596/ | 93.4 | A | 115.3 | P | 129.9 | A | 54.2 |
| | | gi = 4318626/ug = Mm 31114/len = 339/STRA = for | | | | | | | |
| 5 | 92724_at | Cluster Incl AI183202 uc50h11.r1 Mus musculus cDNA, 5 end/clone = IMAGE-1429125/clone_end = 5/gb = AI183202/ | 38.8 | A | 78.8 | A | 106.6 | A | 108.8 |
| | | gi = 3733840/ug = Mm.46512/len = 314/STRA = for | | | | | | | |
| 5 | 93858_at | Cluster Incl M33266 Murine macrophage interferon inducible protein 10 (IP-10) mRNA, complete cds/cds = (40,336)/ | 103.3 | P | 16.5 | A | 55.3 | P | 126 |
| | | gb = M33266/gi = 198444/ug = Mm.877/len = 1063/STRA = rev | | | | | | | |
| 5 | 96090_g_at | Cluster Incl AI255972 ui76a02 x1 Mus musculus cDNA, 3 end/clone = IMAGE-1888298/clone_end = 3/gb = AI255972/ | 10.2 | A | 46.4 | P | 52.7 | A | 45.4 |
| | | gi = 3863497/ug = Mm.26726/len = 511/STRA = for | | | | | | | |
| 5 | 101998_at | Cluster Incl AW125086 UI-M-BH2 1-apx-f-03-0-UI s1 Mus musculus cDNA, 3 end/clone = UI-M-BH2 1-apx-f-03-0-UI/ | 95.6 | P | 93.4 | A | 55.3 | A | 53.8 |
| | | clone_end = 3/gb = AW125086/gi = 6100616/ug = Mm 9860/len = 461/STRA = for | | | | | | | |
| 5 | 102030_at | Cluster Incl AF026032 X-linked nuclear protein/cds = (220,7650)/gb = AF026032/gi = 3002557/ug = Mm 10141/len = 7673/ | 63 | P | 33.6 | A | 35.1 | A | 20.3 |
| | | STRA = for | | | | | | | |
| 5 | 102761_at | Cluster Incl AF041060 Mus musculus co-chaperone mt-GrpE#2 mRNA, nuclear gene encoding mitochondrial protein, | 204.1 | A | 101.4 | A | 118.6 | A | 179.6 |
| | | partial cds/cds = (0,671)/gb = AF041060/gi = 3411071/ug = Mm 12959/len = 1445/STRA = for | | | | | | | |
| 5 | 103797_at | Cluster Incl AB019388: Cell divison cycle 7-like 1/cds-(86,1780)/gb-AB019388/gi = 3869101/ug = Mm 20842/len = 2708/ | 61.6 | A | 53.1 | A | 46.7 | P | 44.3 |
| | | STRA = rev | | | | | | | |
| 5 | 98514_at | Cluster Incl AF004833 Mus musculus tissue factor pathway inhibitor (TFPI) mRNA, complete cds/cds = (104,1024)/ | 84 | A | 107.1 | P | 82.6 | A | 77.6 |
| | | gb = AF004833/gi = 2897076/ug = Mm 3601/len = 1288/STRA = for | | | | | | | |
| 5 | 99465_at | Cluster Incl AJ132922: Mus musculus mRNA for protein encoded by methyl-CpG-binding protein 2/cds = (35,1489)/ | 154.8 | A | 164.1 | P | 136.4 | A | 93.2 |
| | | gb = AJ132922/gi = 5419961/ug = Mm.40984/len = 10087/STRA = for | | | | | | | |
| 5 | 99507_at | Cluster Incl M21247 Uncoupling protein, mitochondrial/cds = (0,923)/gb = M21247/gi = 202268/ug = Mm 4177/len = 924/ | 49.3 | A | 39.8 | A | 88.2 | A | 126.8 |
| | | STRA = for | | | | | | | |
| 5 | 100431_at | Cluster Incl U42467.Leptin receptor/cds = (60,2744)/gb = U42467/gi = 1139592/ug = Mm 4756/len = 4560/STRA = for | 19.9 | A | 21.4 | A | 54.6 | P | −1.8 |
| 5 | 100876_at | Cluster Incl AI841076 UI-M-AH0-acz-a-07-0-UI s1 Mus musculus cDNA, 3 end/clone = UI-M-AH0-acz-a-07-0-UI/ | 49.3 | P | −93.1 | A | 19.5 | P | −13.4 |
| | | clone_end = 3/gb = AI841076/gi = 5475289/ug = Mm 5264/len = 424/STRA = for | | | | | | | |
| 5 | 103444_at | Cluster Incl AI272489.uk06e09 y1 Mus musculus cDNA, 5 end/clone = IMAGE-1958632/clone_end = 5/gb = AI272489/ | 102 | P | 54.6 | A | 80.9 | A | 73.2 |
| | | gi = 3894757/ug = Mm 21492/len = 459/STRA = for | | | | | | | |
| 5 | 103763_at | Cluster Incl AW121855 UI-M-BH2 3-aoi-b-08-0-UI s1 Mus musculus cDNA, 3 end/clone = UI-M-BH2.3-aoi-b-08-0-UI/ | 126 | M | 189.1 | P | 48.2 | A | 92.5 |
| | | clone_end = 3/gb = AW121855/gi = 6097229/ug = Mm.42637/len = 462/STRA = for | | | | | | | |
| 5 | 104478_at | Cluster Incl U27177 Retinoblastoma-like 1 (p107)/cds = (63,3254)/gb = U27177/gi = 1871224/ug = Mm 2994/len = 4863/ | 153 | P | 121.9 | P | 0.1 | A | 79 |
| | | STRA = for | | | | | | | |
| 6 | 92585_at | Cluster Incl L02526 Protein kinase, mitogen activated, kinase 1, p45/cds = (40,1221)/gb = L02526/gi = 199123/ug = Mm.1059/ | 130.1 | P | 165 | P | 154.8 | P | 338.6 |
| | | len = 2119/STRA = for | | | | | | | |
| 6 | 92586_at | Cluster Incl X57024: Glutamate dehydrogenase/cds = (76,1752)/gb = X57024/gi = 51081/ug = Mm 10600/len = 2942/ | 497.2 | P | 558.5 | P | 741.3 | P | 535.1 |
| | | STRA = for | | | | | | | |
| 6 | 92597_s_at | Cluster Incl U13838 Mus musculus vacuolar adenosine triphosphatase subunit B gene, complete cds/cds = (0,1535)/ | 417.8 | P | 363.1 | P | 380.6 | P | 418.1 |
| | | gb = U13838/gi = 1184660/ug = Mm.10727/len = 1536/STRA = for | | | | | | | |
| 6 | 92828_at | Cluster Incl AB004789 Mus musculus mRNA for dolichol-phosphate-mannose synthase, complete cds/cds = (0,782)/ | 451.4 | P | 467.3 | P | 525 | P | 482.8 |
| | | gb = AB004789/gi = 3073772/ug = Mm.12969/len = 789/STRA = for | | | | | | | |
| 6 | 92847_s_at | Cluster Incl X56831: Mannose-6-phosphate receptor, cation dependent/cds = (98,934)/gb = X56831/gi = 53205/ug = Mm 1358/ | 505.4 | P | 511.7 | P | 607.5 | P | 698.8 |
| | | len = 1395/STRA = for | | | | | | | |
| 6 | 92854_at | Cluster Incl D50500 Mouse mRNA for Rab 11, partial sequence/cds = (0,291)/gb = D50500/gi = 1742912/ug = Mm.1387/ | 1964.2 | P | 1943.4 | P | 1857 | P | 2534.6 |
| | | len = 464/STRA = for | | | | | | | |
| 6 | 93037_i_at | Cluster Incl M69260 Lipocortin 1/cds = (14,1054)/gb = M69260/gi = 198844/ug = Mm 14860/len = 1340/STRA = for | 1209.3 | P | 1224.3 | P | 1201.4 | P | 1477.1 |
| 6 | 93043_at | Cluster Incl D50463 Mouse SDR1 mRNA, complete cds/cds = (27,872)/gb = D50463/gi = 1747303/ug = Mm 15125/len = 1900/ | 694.8 | P | 722.4 | P | 585.6 | P | 732.9 |
| | | STRA = for | | | | | | | |
| 6 | 93066_at | Cluster Incl D16195 Granulin/cds = (0,1769)/gb = D16195/gi = 286067/ug = = Mm 1568/len = 1770/STRA = for | 390.6 | P | 432.2 | P | 551.7 | P | 399.7 |
| 6 | 93261_at | Cluster Incl AJ000990 Mus musculus mRNA for legumain/cds = (94,1401)/gb = AJ000990/gi = 3676226/ug = Mm 17185/ | 313.7 | P | 543.7 | P | 432.7 | P | 514.8 |
| | | len = 1889/STRA = for | | | | | | | |
| 6 | 93290_at | Cluster Incl U35374 Mus musculus purine nucleoside phosphorylase (Np-b) mRNA, complete cds/cds = (0,869)/gb = U35374/ | 570.4 | P | 533.2 | P | 575.2 | P | 948.1 |
| | | gi = 114566/ug = Mm 17932/len = 870/STRA = for | | | | | | | |
| 6 | 93309_at | Cluster Incl U42386: Fibroblast growth factor Inducible 14/cds = (880,1065)/gb = U42386/gi = 1353710/ug = Mm.18459/ | 440.9 | P | 483.3 | P | 534.9 | P | 497.5 |
| | | len = 2610/STRA = for | | | | | | | |
| 6 | 93320_at | Cluster Incl AF017175: Camitine palmitoyltransferase 1, liver/cds = (0,2296)/gb = AF017175/gi = 2388721/ug = Mm.18522/ | 471.2 | P | 431.2 | P | 902.7 | P | 654.7 |
| | | len = 4414/STRA = for | | | | | | | |
| 6 | 93347_at | Cluster Incl Z22819: RAB24, member RAS oncogene family/cds = (0,611)/gb = Z22819/gi = 438163/ug = Mm 18800/len = 660/ | 342.5 | P | 336.3 | P | 398.4 | P | 472.7 |
| | | STRA = for | | | | | | | |
| 6 | 93364_at | Cluster Incl X59990: Calenin alpha 1/cds = (72,2792)/gb = X59990/gi = 49934/ug = Mm 18962/len = 3713/STRA = for | 672.9 | P | 780.6 | P | 908.5 | P | 867.7 |
| 6 | 93497_at | Cluster Incl K02782: Mouse complement component C3 mRNA, alpha and beta subunits, complete cds/cds = (56,5047)/ | 443.8 | P | 748.9 | P | 715.1 | P | 869.7 |
| | | gb = K02782/gi = 192278/ug = Mm 19131/len = 5067/STRA = for | | | | | | | |
| 6 | 93498_s_at | Cluster Incl M97216: Mouse DNA-binding protein mRNA, complete cds/cds = (0,1535)/gb = M97216/gi = 515628/ | 1670.4 | P | 1393.8 | P | 1579.9 | P | 1773.5 |
| | | ug = Mm 19133/len = 1536/STRA = for | | | | | | | |
| 6 | 93499_at | Cluster Incl U16740: Mus musculus capping protein alpha 1 subunit mRNA, partial cds/cds = (0,856)/gb = U16740/ | 1318.7 | P | 1147.6 | P | 1061.5 | P | 1317.4 |
| | | gi = 595916/ug = Mm 19142/len = 2893/STRA = for | | | | | | | |
| 6 | 93818_g_at | Cluster Incl AB025218: Mus musculus mRNA for Sid394p, complete cds/cds = (33,638)/gb = AB025218/gi = 5931556/ | 1835 | P | 1524 | P | 1930.7 | P | 1947.3 |
| | | ug = Mm 21507/len = 677/STRA = for | | | | | | | |
| 6 | 93819_at | Cluster Incl AI847269: UI-M-AI1-afq-h-06-0-UI.s1 Mus musculus cDNA, 3 end/clone = UI-M-AI1-afq-h-06-0-UI/clone_end = 3/ | 1998.2 | P | 2787.9 | P | 2652.7 | P | 2392.9 |
| | | gb = AI847269/gi = 5491175/ug = Mm.21507/len = 287/STRA = for | | | | | | | |
| 6 | 93994_at | Cluster Incl AW212131: uo82g04.x1 Mus musculus cDNA, 3 end/clone = IMAGE-2649078/clone_end = 3/gb = AW212131/ | 339.2 | P | 470.8 | P | 354.8 | P | 377.3 |
| | | gi = 6518137/ug = Mm 21754/len = 566/STRA = for | | | | | | | |
| 6 | 94002_at | Cluster Incl AI849838: UI-M-AL1-aho-d-08-0-UI.s1 Mus musculus cDNA, 3 end/clone = UI-M-AL1-aho-d-08-0-UI/ | 538.4 | P | 482.4 | P | 485 | P | 472.8 |
| | | clone_end = 3/gb = AI849838/gi = 5493737/ug = Mm 21771/len = 417/STRA = for | | | | | | | |
| 6 | 94003_at | Cluster Incl AI848510: UI-M-AP1-agf-d-06-0-UI.s2 Mus musculus cDNA, 3 end/clone = UI-M-AP1-agf-d-06-0-UI/ | 731.4 | P | 808 | P | 858.1 | P | 857.4 |
| | | clone_end = 3/gb = AI848510/gi = 5492416/ug = Mm 21773/len = 420/STRA = for | | | | | | | |
| 6 | 94041_at | Cluster Incl AI840643: UI-M-AM0-adq-g-08-0-UI.s1 Mus musculus cDNA, 3 end/clone = UI-M-AM0-adq-g-08-0-UI/ | 795.3 | P | 822.3 | P | 842.4 | P | 817.2 |
| | | clone_end = 3/gb = AI840643/gi = 5474851/ug = Mm.21953/len = 517/STRA = for | | | | | | | |
| 6 | 95412_at | Cluster Incl U49112: Mus musculus calcium-binding protein ALG-2 (ALG-257) mRNA, complete cds/cds = (56,631)/ | 525 | P | 613.2 | P | 669.7 | P | 792.5 |
| | | gb = U49112/gi = 1213519/ug = Mm 24254/len = 1060/STRA = for | | | | | | | |
| 6 | 95428_at | Cluster Incl AA688761: vr07b11.r1 Mus musculus cDNA, 5 end/clone = IMAGE-1111101/clone_end = 5/gb = AA883761/ | 367.9 | P | 317 | P | 319.5 | P | 358.2 |
| | | gi = 2678190/ug = Mm 24456/len = 322/STRA = for | | | | | | | |
| 6 | 95434_at | Cluster Incl AI851740: UI-M-BH0-aim-a-03-0-UI.s1 Mus musculus cDNA, 3 end/clone = UI-M-BH0-aim-a-03-0-UI/ | 1338.6 | P | 1322.8 | P | 1412.4 | P | 1675.1 |
| | | clone_end = 3/gb = AI851740/gi = 5495641/ug = Mm 24498/len = 373/STRA = for | | | | | | | |
| 6 | 95444_at | Cluster Incl AW122274: UI-M-BH2.2-aos-a-04-0-UI.s1 Mus musculus cDNA, 3 end/clone = UI-M-BH2.2-aos-a-04-0-UI/ | 945.6 | P | 901.5 | P | 895.1 | P | 1120.5 |
| | | clone_end = 3/gb = AW122274/gi = 6097751/ug = Mm 24584/len = 261/STRA = for | | | | | | | |
| 6 | 95489_at | Cluster Incl AW047953: UI-M-BH1-aln-f-07-0-UI.s1 Mus musculus cDNA, 3 end/clone = UI-M-BH1-aln-f-07-0-UI/ | 249.4 | P | 277.3 | P | 222.2 | P | 277.7 |
| | | clone_end = 3/gb = AW047953/gi = 5908482/ug = Mm 25044/len = 331/STRA = for | | | | | | | |
| 6 | 95507_at | Cluster Incl AB025048: Mus musculus mRNA for Sid6061p, complete cds/cds = (69,1025)/gb = AB025048/gi = 5931550/ | 292.9 | P | 210.4 | P | 229 | P | 302.3 |
| | | ug = Mm 25198/len = 1912/STRA = for | | | | | | | |
| 6 | 96003_at | Cluster Incl AW048332: UI-M-BH1-akk-c-05-0-UI.s1 Mus musculus cDNA, 3 end/clone = UI-M-BH1-akk-c-05-0-UI/ | 974.4 | P | 756.1 | P | 1100.2 | P | 968.8 |
| | | clone_end = 3/gb = AW048332/gi = 5908861/ug = Mm 25339/len = 333/STRA = for | | | | | | | |
| 6 | 96007_at | Cluster Incl AI835359: UI-M-AQ0-aah-g-05-0-UI s1 Mus musculus cDNA, 3 end/clone = UI-M-AQ0-aah-g-05-0-UI/ | 1650.9 | P | 1696.6 | P | 1557.5 | P | 1660.3 |
| | | clone_end = 3/gb = AI835359/gi = 5469572/ug = Mm 25393/len = 305/STRA = for | | | | | | | |
| 6 | 96120 at | Cluster Incl AW124750: UI-M-BH2.1-aqa-e-04-0-UI s1 Mus musculus cDNA, 3 end/clone = UI-M-BH2.1-aqa-e-04-0-UI/ | 613.7 | P | 656.9 | P | 788 | P | 605.4 |
| | | clone_end = 3/gb = AW124750/gi = 6100280/ug = Mm 2701/len = 184/STRA = for | | | | | | | |
| 6 | 96607_at | Cluster Incl AW124902: UI-M-BH2 1-apu-d-12-0-UI s1 Mus musculus cDNA, 3 end/clone = UI-M-BH2.1-apu-d-12-0-UI/ | 724.1 | P | 749.9 | P | 706.1 | P | 784.8 |
| | | clone_end = 3/gb = AW124902/gi = 6100432/ug = Mm 27065/len = 281/STRA = for | | | | | | | |
| 6 | 96613_at | Cluster Incl AI852095: UI-M-BH0-aja-h-02-0-UI.s1 Mus musculus cDNA, 3 end/clone = UI-M-BH0-aja-h-02-0-UI/ | 490.2 | P | 550 | P | 536.6 | P | 496.4 |
| | | clone_end = 3/gb = AI852095/gi = 5496001/ug = Mm 27107/len = 381/STRA = for | | | | | | | |
| 6 | 96675_at | Cluster Incl AW124245: UI-M-BH2.1-aph-f-06-0-UI.s1 Mus musculus cDNA, 3 end/clone = UI-M-BH2.1-aph-f-06-0-UI/ | 565.5 | P | 735.8 | P | 676.8 | P | 709.7 |
| | | clone_end = 3/gb = AW124245/gi = 6099775/ug = Mm 27404/len = 375/STRA = for | | | | | | | |
| 6 | 96701_at | Cluster Incl U35833: Mus musculus anthracycline-associated resistance (Arx) mRNA, complete cds/cds = (20,1936)/ | 1121.7 | P | 1180.1 | P | 1039.1 | P | 1121.7 |
| | | gb = U35833/gi = 4096673/ug = Mm.27560/len = 2525/STRA = for | | | | | | | |
| 6 | 97262_at | Cluster Incl AW050305: UI-M-BH1-ans-g-11-0-UI.s1 Mus musculus cDNA, 3 end/clone = UI-M-BH1-ans-g-11-0-UI/ | 673.9 | P | 819.5 | P | 815.8 | P | 772.8 |
| | | clone_end = 3/gb = AW050305/gi = 5910834/ug = Mm 27899/len = 342/STRA = for | | | | | | | |
| 6 | 97829_at | Cluster Incl AI838053: UI-M-AL0-abv-c-03-0-UI s1 Mus musculus cDNA, 3 end/clone = UI-M-AL0-abv-c-03-0-UI/ | 228.7 | P | 288.4 | P | 358.7 | P | 485.2 |
| | | clone_end = 3/gb = AI836053/gi = 5472266/ug = Mm 28219/len = 367/STRA = for | | | | | | | |
| 6 | 97844_at | Cluster Incl U67187: Mus musculus G protein signaling regulator RGS2 (rgs2) mRNA, complete cds/cds = (20,655)/ | 271.2 | P | 219.2 | P | 249.4 | P | 250.4 |
| | | gb = U67187/gi = 1911236/ug = Mm 28262/len = 1240/STRA = for | | | | | | | |
| 6 | 97904_at | Cluster Incl AW 123953: UI-M-BH2.3-aqc-d-06-0-UI.s1 Mus musculus cDNA, 3 end/clone = UI-M-BH2.3-aqc-d-06-0-UI/ | 2097.7 | P | 2182.6 | P | 2149.2 | P | 2258.6 |
| | | clone_end = 3/gb = AW123953/gi = 6099483/ug = Mm.28464/len = 382/STRA = for | | | | | | | |
| 6 | 94452_g_at | Cluster Incl AI787627: ul20f06.y1 Mus musculus cDNA, 5 end/clone = IMAGE-2088131/clone_end = 5/gb = AI787627/ | 792.9 | P | 775.3 | P | 766.6 | P | 806.1 |
| | | gi = 5335343/ug = Mm 28786/len = 450/STRA = for | | | | | | | |
| 6 | 94461_at | Cluster Incl AI852144: UI-M-BH0-ajb-e-09-0-UI.s1 Mus musculus cDNA, 3 end/clone = UI-M-BH0-ajb-e-09-0-UI/ | 168.1 | P | 132.5 | P | 161.7 | P | 179.1 |
| | | clone_end = 3/gb = AI652144/gi = 5496055/ug = Mm 28830/len = 425/STRA = for | | | | | | | |
| 6 | 94502_at | Cluster Incl AW125724: UI-M-BH2 2-aql-d-12-0-UI.s1 Mus musculus cDNA, 3 end/clone = UI-M-BH2.2-aql-d-12-0-UI/ | 157.6 | P | 199.4 | P | 177.1 | P | 182.7 |
| | | clone_end = 3/gb = AW125724/gi = 8101294/ug = Mm 2895/len = 460/STRA = for | | | | | | | |
| 6 | 95070_at | Cluster Incl AW125874: UI-M-BH2 2-aqk-f-09-0-UI s1 Mus musculus cDNA, 3 end/clone = UI-M-BH2.2-aqk-f-09-0-UI/ | 289.1 | P | 243.4 | P | 352.2 | P | 392.3 |
| | | clone_end = 3/gb = AW125874/gi = 6101404/ug = Mm 29192/len = 266/STRA = for | | | | | | | |
| 6 | 95086_at | Cluster Incl AI835392: UI-M-AQ0-aaj-b-10-0-UI.s1 Mus musculus cDNA, 3 end/clone = UI-M-AQ0-aaj-b-10-0-UI/ | 945 | P | 1047.9 | P | 981.9 | P | 1085.4 |
| | | clone_end = 3/gb = AI835392/gi = 5469605/ug = Mm 29259/len = 221/STRA = for | | | | | | | |
| 6 | 95140_at | Cluster Incl AI842068: UI-M-AN1-afg-b-01-0-UI.s1 Mus musculus cDNA, 3 end/clone = UI-M-AN1-afg-b-01-0-UI/ | 608 | P | 475.6 | P | 838.9 | P | 548.3 |
| | | clone_end = 3/gb = AI842068/gi = 5476281/ug = Mm 29436/len = 397/STRA = for | | | | | | | |
| 6 | 95148_at | Cluster Incl AB020202: Mus musculus mRNA for adenylate kinase isozyme 2, complete cds/cds = (42,740)/gb = AB020202/ | 343.1 | P | 515.6 | P | 389 | P | 478.8 |
| | | gi = 4760597/ug = Mm.29460/len = 983/STRA = for | | | | | | | |
| 6 | 95654_at | Cluster Incl AF109905: Mus musculus major histocompatibility locus class III regions Hsc70t gene, partial cds; smRNP, | 1230.6 | P | 1554 | P | 1427.9 | P | 1819.2 |
| | | G7A, NG23, MutS homolog, CLCP, NG24, NG25, and NG26 genes, complete cds, and unknown genes/cds = (0,725)/ | | | | | | | |
| | | gb = AF109905/gi = 3986751/ug = Mm 29/STRA = for | | | | | | | |
| 6 | 95708_at | Cluster Incl AI843466 UI-M-AQ1-aec-g-01-0-UI s1 Mus musculus cDNA, 3 end/clone = UI-M-AQ1-aoc-g-01-0-UI/ | 1303.9 | P | 1271.9 | P | 1234.3 | P | 1520.9 |
| | | clone_end = 3/gb = AI843466/gi = 5477684/ug = Mm 29702/len = 325/STRA = for | | | | | | | |
| 6 | 96281_at | Cluster Incl AW121246 UI-M-BH2 3-aok-g-05-0-UI s1 Mus musculus cDNA, 3 end/clone = UI-M-BH2 3-aok-g-05-0-UI/ | 2004.4 | P | 1881.5 | P | 2259.5 | P | 2278.6 |
| | | clone_end = 3/gb = AW121246/gi = 6096579/ug = Mm 29868/len = 355/STRA = for | | | | | | | |
| 6 | 96333_g_at | Cluster Incl AW259199.um91a03 y1 Mus musculus cDNA, 5 end/clone = lMAGE-2332108/clone_end = 5/gb = AW259199/ | 580.7 | P | 434.3 | P | 373.6 | P | 491.7 |
| | | gi = 6632180/ug = Mm 29971/len = 411/STRA = for | | | | | | | |
| 6 | 96348_at | Cluster Incl AW121217.UI-M-BH2 3-aok-d-10-0-UI s1 Mus musculus cDNA, 3 end/clone = UI-M-BH2 3-aok-d-10-0-UI/ | 936.1 | P | 1359 | P | 1683.9 | P | 1192.3 |
| | | clone_end = 3/gb = AW121217/gi = 6096550/ug = Mm 29998/len = 454/STRA = for | | | | | | | |
| 6 | 96353_at | Cluster Incl AW125346 UI-M-BH2 1-apy-h-02-0-UI s1 Mus musculus cDNA, 3 end/clone = UI-M-BH2 1-apy-h-02-0-UI/ | 461.6 | P | 579.3 | P | 426 | P | 568 |
| | | clone_end = 3/gb = AW125346/gi = 6100876/ug = Mm 30005/len = 382/STRA = for | | | | | | | |
| 6 | 96913_at | Cluster Incl AW122615 UI-M-BH2.2 aol-b-06-0-UI s1 Mus musculus cDNA, 3 end/clone = UI-M-BH2 2-aol-b-06-0-UI/ | 969.5 | P | 1032.3 | P | 1327.1 | P | 1035.9 |
| | | clone_end = 3/gb = AW1226l5/gi = 6098142/ug = Mm.30147/len = 244/STRA = for | | | | | | | |
| 6 | 96936_at | Cluster Incl AI020792 ub04g10 r1 Mus musculus cDNA, 5 end/clone = IMAGE-1366050/clone_end = 5/gb = AI020792/ | 380.2 | P | 429.1 | P | 374.4 | P | 433.5 |
| | | gi = 3235128/ug = Mm 30183/len = 421/STRA = for | | | | | | | |
| 6 | 97486_at | Cluster Incl AA693246 vr58e09.s1 Mus musculus cDNA, 5 end/clone = lMAGE-1124872/clone_end = 5/gb = AA693246/ | 835.7 | P | 868.8 | P | 902.5 | P | 834.7 |
| | | gi = 2694184/ug = Mm 30926/len = 397/STRA = for | | | | | | | |
| 6 | 98101_at | Cluster Incl AI851798 UI-M-BH0-ais-a-06-0-UI.s1 Mus musculus cDNA, 3 end/clone = UI-M-BH0-als-a-06-0-UI/ | 351.2 | P | 359 | P | 316.3 | P | 339.5 |
| | | clone_end = 3/gb = AI851798/gi = 5495704/ug = Mm.34772/len = 300/STRA = for | | | | | | | |
| 6 | 98102_at | Cluster Incl M76727: Pyruvate dehydrogenase E1 alpha subunit/cds = (111,1283)/gb = M76727/gi = 2002768/ug = Mm 34775/ | 907.4 | P | 1177.8 | P | 1356 | P | 1186.2 |
| | | len = 1446/STRA = for | | | | | | | |
| 6 | 98521_at | Cluster Incl AI847972 UI-M-AI1-all-h-04-0-UI s1 Mus musculus cDNA, 3 end/clone = UI-M-AI1-all-h-04-0-UI/clone_end = 3/ | 584.7 | P | 683.4 | P | 609.4 | P | 639.5 |
| | | gb = AI847972/gi = 5491878/ug = Mm.35808/len = 172/STRA = for | | | | | | | |
| 6 | 98525_f_at | Cluster Incl AJ007909.Mus musculus mRNA for erythroid differentiation regulator, partial/cds = (154,687)/gb = AJ007909/ | 1409.2 | P | 884.9 | P | 1231.1 | P | 1408.3 |
| | | gi = 3336885/ug = Mm.35829/len = 715/STRA = for | | | | | | | |
| 6 | 99158_at | Cluster Incl U58888 Mus musculus SH3-containing protein SH3P2 mRNA, partial cds/cds = (0,755)/gb = U58888/ | 557.4 | P | 656.3 | P | 630.9 | P | 617.5 |
| | | gi = 1407662/ug = Mm 4165/len = 1041/STRA = for | | | | | | | |
| 6 | 99169_at | Cluster Incl AW122165 UI-M-BH2.2-aor-g-07-0-UI s1 Mus musculus cDNA, 3 end/clone = UI-M-BH2.2-aor-g-07-0-UI/ | 522.8 | P | 568.5 | P | 753.9 | P | 579.8 |
| | | clone_end = 3/gb = AW122165/gi = 6097606/ug = Mm.41705/len = 204/STRA = for | | | | | | | |
| 6 | 99587_at | Cluster Incl X89650 RAB7, member RAS oncogene family/cds = (156,779)/gb = X89650/gi = 1050550/ug = Mm.4268/ | 665 | P | 1237.4 | P | 1268.6 | P | 1251.4 |
| | | len = 2089/STRA = for | | | | | | | |
| 6 | 99610_at | Cluster Incl X93357 Synovial sarcoma, translocated to X Chromosome/cds = (179,1435)/gb = X93357/gi = 1072383/ | 466.8 | P | 674.5 | P | 578.4 | P | 539.9 |
| | | ug = Mm 4296/len = 3107/STRA = for | | | | | | | |
| 6 | 100572_at | Cluster Incl AI851289 UI-M-BH0-akf-f-05-0-UI s1 Mus musculus cDNA, 3 end/clone = UI-M-BH0-akf-f-05-0-UI/ | 530.9 | P | 542 | P | 532.3 | P | 506.7 |
| | | clone_end = 3/gb = AI851289/gi = 5495195/ug = Mm.5885/len = 449/STRA = for | | | | | | | |
| 6 | 100595_at | Cluster Incl AF035644: Mus musculus potentially prenylated protein tyrosine phosphatase mPRL-2 (Prl2) mRNA, complete | 1384.8 | P | 1724.8 | P | 1524.8 | P | 1634 |
| | | cds/cds = (179,682)/gb = AF035644/gi = 2992629/ug = Mm 6355/len = 723/STRA = for | | | | | | | |
| 6 | 100603_at | Cluster Incl AF063231: Dynein, cytoplasmic, intermediate chain 2/cds = (77,1915)/gb = AF063231/gi = 3493590/ | 213.6 | P | 157.8 | P | 181.4 | P | 213.2 |
| | | ug = Mm 6441/len = 2458/STRA = for | | | | | | | |
| 6 | 101040_at | Cluster Incl D38117: Mus musculus mRNA for m-calpain large subunit, complete cds/cds = (170,2272)/gb = D38117/ | 575.4 | P | 709.4 | P | 734 | P | 859.2 |
| | | gi = 2570157/ug = Mm 6958/len = 3225/STRA = for | | | | | | | |
| 6 | 101982_at | Cluster Incl X98475 M musculus VASP gene/cds = (0,1130)/gb = X98475/gi = 1617401/ug = Mm.9684/len = 1131/STRA = for | 638.1 | P | 587.1 | P | 811.2 | P | 1124.1 |
| 6 | 102407_at | Cluster Incl M68898: Mast cell protease 5/cds = (5,748)/gb = M68898/gi = 199028/ug = Mm 1252/len = 960/STRA = for | 1091.5 | P | 1881.5 | P | 3483.1 | P | 1732.6 |
| 6 | 102790_at | Cluster Incl AB016490 Mus musculus mJTB mRNA, complete cds/cds = (306,746)/gb = AB016490/gi = 3721891/ | 1278.4 | P | 1233.7 | P | 1154.2 | P | 1252.8 |
| | | ug = Mm 13912/len = 1273/STRA = for | | | | | | | |
| 6 | 103018_at | Cluster Incl AF096868 Mus musculus transcytosis associated protein p115 mRNA, partial cds/cds = (0,2827)/gb = AF096868/ | 202.9 | P | 240.3 | P | 157.2 | P | 252.8 |
| | | gi = 3860050/ug = Mm 15868/len = 3690/STRA = for | | | | | | | |
| 6 | 103899_at | Cluster Incl AA690863 vt32b05 r1 Mus musculus cDNA, 5 end/clone = IMAGE-1164753/clone_end = 5/gb = AA690863/ | 262 | P | 251.3 | P | 249.9 | P | 351.2 |
| | | gi = 2691799/ug = Mm 21313/len = 289/STRA = for | | | | | | | |
| 6 | 104315_at | Cluster Incl AI846773 UI-M-AO1-ael-f-02-0-UI s1 Mus musculus cDNA, 3 end/clone = UI-M-AO1-ael-f-02-0-UI/ | 320.9 | P | 259 | P | 341.2 | P | 354.1 |
| | | clone_end = 3/gb = AI846773/gi = 5490679/ug = Mm.22413/len = 322/STRA = for | | | | | | | |
| 6 | 104350_at | Cluster Incl AI050321 ub29c11 r1 Mus musculus cDNA, 5 end/clone = IMAGE-1379158/clone_end = 5/gb = AI050321/ | 298.5 | P | 388.4 | P | 370.4 | P | 403 |
| | | gi = 3299438/ug = Mm.22550/len = 504/STRA = for | | | | | | | |
| 6 | 104612_g_at | Cluster Incl AI854008 UI-M-BH0-aiv-h-12-0-UI s1 Mus musculus cDNA, 3 end/clone = UI-M-BH0-aiv-h-12-0-UI/ | 282.2 | P | 219.4 | P | 277.2 | P | 251.3 |
| | | clone_end = 3/gb = AI854008/gi = 5497914/ug = Mm.24158/len = 494/STRA = for | | | | | | | |
| 6 | 94369_at | Cluster Incl AW123026 UI-M-BH2 1-apb-f-07-0-UI s1 Mus musculus cDNA, 3 end/clone = UI-M-BH2 1-apb-f-07-0-UI/ | 149.5 | P | 134 | P | 114 | P | 219.7 |
| | | clone_end = 3/gb = AW123026/gi = 6098556/ug = Mm.25336/len = 442/STRA = for | | | | | | | |
| 6 | 94408_at | Cluster Incl U47008: Ngfi-A binding protein 1/cds = (136,1596)/gb = U47008/gi = 1197670/ug = Mm 25903/len = 1796/ | 1062.1 | P | 1082.1 | P | 1239.1 | P | 1250.5 |
| | | STRA = for | | | | | | | |
| 6 | 94427_at | Cluster Incl AI841737 UI-M-AL0-abo-g-05-0-UI.s1 Mus musculus cDNA, 3 end/clone = UI-M-AL0-abo-g-05-0-UI/ | 205.3 | P | 271.3 | P | 240.5 | P | 261.3 |
| | | clone_end = 3/gb = AI841737/gi = 5475950/ug = Mm.26422/len = 372/STRA = for | | | | | | | |
| 6 | 94963_at | Cluster Incl AI462105 ub70b01 x1 Mus musculus cDNA, 3 end/clone = IMAGE-1383049/clone_end = 3/gb = AI462105/ | 1152.5 | P | 1183.5 | P | 1082.4 | P | 1247.2 |
| | | gi = 4316135/ug = Mm.27206/len = 422/STRA = for | | | | | | | |
| 6 | 95608_at | Cluster Incl AI851255 UI-M-BH0-akf-c-03-0-UI.s1 Mus musculus cDNA, 3 end/clone = UI-M-BH0-akf-c-03-0-UI/ | 120.6 | P | 183.2 | P | 159.9 | P | 253.8 |
| | | clone_end = 3/gb = AI851255/gi = 5495161/ug = Mm.27986/len = 427/STRA = rev | | | | | | | |
| 6 | 96239_at | Cluster Incl AA815503 vp20d05.r1 Mus musculus cDNA, 5 end/clone = IMAGE-1069161/clone_end = 5/gb = AA815503/ | 162.6 | P | 154.5 | P | 103.3 | P | 232.4 |
| | | gi = 2885099/ug = Mm.28816/len = 477/STRA = for | | | | | | | |
| 6 | 97939_at | Cluster Incl AW125079: UI-M-BH2.1-apx-e-08-0-UI.s1 Mus musculus cDNA, 3 end/clone = UI-M-BH2.1-apx-e-08-0-UI/ | 783.7 | P | 752.5 | P | 645.2 | P | 839.7 |
| | | clone_end = 3/gb = AW125079/gi = 6100609/ug = Mm.30265/len = 316/STRA = for | | | | | | | |
| 6 | 97949_at | Cluster Incl M16238: Fibrinogen-like protein 2/cds = (60,1358)/gb = M16238/gi = 193305/ug = Mm 3040/len = 3772/STRA = for | 334.5 | P | 261 | P | 194.8 | P | 461.1 |
| 6 | 98466_r_at | Cluster Incl M31419 Interferon activated gene 204/cds = (127,2049)/gb = M31419/gi = 198399/ug = Mm 34817/len = 2353/ | 190.2 | P | 187.1 | P | 190.6 | P | 453.9 |
| | | STRA = for | | | | | | | |
| 6 | 99051_at | Cluster Incl M36579 S100 calcium-binding protein A4/cds = (0,164)/gb = M36579/gi = 199816/ug = Mm 3925/len = 302/ | 431.3 | P | 733.7 | P | 318.5 | P | 605.1 |
| | | STRA = for | | | | | | | |
| 6 | 99503_at | Cluster Incl AW045204 UI-M-BH1-alh-c-10-0-UI s1 Mus musculus cDNA, 3 end/clone = UI-M-BH1-alh-c-10-0-UI/ | 161.4 | P | 158 | P | 222.2 | P | 419.8 |
| | | clone_end = 3/gb = AW045204/gi = 5905733/ug = Mm.41714/len = 405/STRA = for | | | | | | | |
| 6 | 99996_at | Cluster Incl Y07941: M musculus mRNA for plakophilin 1/cds = (12,2198)/gb = Y07941/gi = 1707593/ug = Mm 4494/len = 4504/ | 1173.1 | P | 1410.7 | P | 1737 | P | 1391.3 |
| | | STRA = rev | | | | | | | |
| 6 | 101867_at | Cluster Incl U11680 Mus musculus glycerol-3-phosphate acyltransferase gene, nuclear gene encoding mitochondrial/ | 509.8 | P | 666.3 | P | 643.1 | P | 673.7 |
| | | protein, 5 region and partial cds/cds = (411,425)/gb = U11680/gi = 786006/ug = Mm 87773/len = 426/STRA = rev | | | | | | | |
| 6 | 101886_f_at | Cluster Incl X52490 Synthetic D(b) mRNA for D(b) glycoprotein (alpha chain) and beta(2) microglobulin/cds = (9,1097)/ | 2303.5 | P | 1283.7 | P | 1144.4 | P | 2150 |
| | | gb = X52490/gi = 50671/ug = Mm.88493/len = 1102/STRA = rev | | | | | | | |
| 6 | 101948_at | Cluster Incl X05212 Mouse mRNA for laminin B1 C-terminus/cds = (0,1489)/gb = X05212/gi = 52861/ug = Mm.963/len = 1640/ | 389.6 | P | 544.2 | P | 555.9 | P | 488.7 |
| | | STRA = for | | | | | | | |
| 6 | 102658_at | Cluster Incl X59769 Interleukin 1 receptor, type II/cds = (92,1324)/gb = X59769/gi = 52670/ug = Mm 1349/len = 1341/ | 129 | P | 196.1 | P | 390.4 | P | 555 |
| | | STRA = for | | | | | | | |
| 6 | 103471_at | Cluster Incl AI194333 ui15d12 r1 Mus musculus cDNA, 5 end/clone = IMAGE-1851383/clone_end = 5/gb = AI194333/ | 292.4 | P | 204.3 | P | 217.1 | P | 224.9 |
| | | gi = 3745540/ug = Mm 21821/len = 459/STRA = for | | | | | | | |
| 6 | 103744_at | Cluster Incl AI852760 UI-M-BH0-ary-b-12-0-UI s1 Mus musculus cDNA,3 end/clone = UI-M-BH0-ary-b-12-0-UI/ | 51.4 | P | 44.5 | P | 60.9 | P | 64.1 |
| | | clone_end = 3/gb = AI852760/gi = 5496666/ug = Mm 24472/len = 417/STRA = for | | | | | | | |
| 6 | 104249_g_at | Cluster Incl AW227650 up 12b09 y1 Mus musculus cDNA, 5 end/clone = IMAGE-2654105/clone_end = 5/gb = AW227650/ | 604.5 | P | 366.5 | P | 580 | P | 561 |
| | | gi = 6556946/ug = Mm.28345/len = 440/STRA = for | | | | | | | |
| 6 | 93721_at | Cluster Incl L12367 Mus musculus adenylyl cyclase-associated protein (CAP) mRNA, complete cds/cds = (64,1488)/ | 392.2 | P | 287.1 | P | 329.3 | P | 407.8 |
| | | gb = L12367/gi = 293316/ug = Mm 8687/len = 1563/STRA = for | | | | | | | |
| 6 | 93871_at | Cluster Incl L32838 Mouse germline interleukin 1 receptor antagonist (IL-1m) gene,complete cds/cds = (0,536)/gb = L32838/ | 244 | P | 176.3 | P | 188.9 | P | 263 |
| | | gi = 487864/ug = Mm.882/len = 537/STRA = for | | | | | | | |
| 6 | 99325_at | Cluster Incl M32502. Wingless-related MMTV integration site 3/cds = (45,1112)/gb = M32502/gi = 198428/ug= Mm 5188/ | 318.9 | P | 137.8 | P | 235.7 | P | 220.9 |
| | | len = 3000/STRA = for | | | | | | | |
| 6 | 101741_at | Cluster Incl AB003305: Proteasome beta type subunit 5, pseudogene/cds = (0,797)/gb = AB003305/gi = 2897807/ | 1225.7 | P | 667.7 | P | 1183 | P | 1316.7 |
| | | ug = Mm 57067/len = 798/STRA = for | | | | | | | |
| 6 | 97593_f_at | Cluster Incl C79694 C79694 Mus musculus cDNA, 3 end/clone = J0070F03/clone_end = 3/gb = C79694/gi = 2520024/ | 367.1 | P | 441.7 | P | 449 | P | 583.6 |
| | | ug = Mm 89577/len = 439/STRA = rev | | | | | | | |
| 6 | 101697_f_at | Cluster Incl AI844607 UI-M-AJ1-ahh-d-12-0-UI s2 Mus musculus cDNA, 3 end/clone = UI-M-AJ1-ahh-d-12-0-UI/ | 190 | P | 269 | P | 268.1 | P | 287.5 |
| | | clone_end = 3/gb = AI844607/gi = 5488513/ug = Mm.39586/len = 121/STRA = rev | | | | | | | |
| 6 | 94084_at | Cluster Incl AI842888 UI-M-AN1-afb-f-12-0-UI s1 Mus musculus cDNA, 3 end/clone = UI-M-AN1-afb-f-12-0-UI/ | 249.2 | P | 146.6 | P | 194.7 | P | 213.3 |
| | | clone_end = 3/gb = AI842888/gi = 5477101/ug = Mm 22192/len = 331/STRA = for | | | | | | | |
| 6 | 96103_f_at | Cluster Incl X92411: RAD23b homolog (S cerevisiae)/cds = (0,1250)/gb = X92411/gi = 1044898/ug = Mm 26879/len = 1251/ | 95.9 | P | 50.1 | P | 170.7 | P | 221.7 |
| | | STRA = for | | | | | | | |
| 6 | 97480_f_at | Cluster Incl U95607 Mus musculus testis specific DNA-homolog mRNA, complete cds/cds = (72,800)/gb = U95607/ | 53.5 | P | 35.4 | P | 63 | P | 59 |
| | | gi = 2286122/ug = Mm 3075/len = 1030/STRA = for | | | | | | | |
| 6 | 98099_at | Cluster Incl AW045760 UI-M-BH1-akt-b-12-0-UI.s1 Mus musculus cDNA, 3 end/clone = UI-M-BH1-akt-b-12-0-UI/ | 143.7 | P | 142.3 | P | 161.8 | P | 159.9 |
| | | clone_end = 3/gb = AW045760/gi = 5906289/ug = Mm. 34750/len = 413/STRA = for | | | | | | | |
| 6 | 100059_at | Cluster Incl M31775 Mouse cytochrome beta-558 mRNA, 3 end/cds = (0,574)/gb = M31775/gi = 192913/ug = Mm 448/ | 657.5 | P | 868.8 | P | 894.8 | P | 903.1 |
| | | len = 677/STRA = for | | | | | | | |
| 6 | 101106_at | Cluster Incl AI853331.UI-M-BH0-air-a-07-0-UI s1 Mus musculus cDNA, 3 end/clone = UI-M-BH0-air-a-07-0-UI/ | 251.8 | P | 284.1 | P | 209.9 | P | 242.6 |
| | | clone_end = 3/gb = AI853331/gi = 5497237/ug = Mm.7514/len = 347/STRA = for | | | | | | | |
| 6 | 102012_at | Cluster Incl AB014485 Mus musculus mRNA for RA70, complete cds/cds = (65,1141)/gb = AB014485/gi = 4730810/ | 314.1 | P | 192.5 | P | 178.1 | P | 551.4 |
| | | ug = Mm 10019/len = 1554/STRA = for | | | | | | | |
| 6 | 102813_f_at | Cluster Incl Aw210346 ul53g12 y1 Mus musculus cDNA, 5 end/clone = IMAGE-2102086/clone_end = 5/gb = AW210346/ | 227.2 | P | 182.9 | P | 173.1 | P | 235 |
| | | gi = 6516286/ug = Mm 14253/len = 431/STRA = for | | | | | | | |
| 6 | 103347_at | Cluster Incl AI852548 UI-M-BH0-arw-f-09-0-UI s1 Mus musculus cDNA, 3 end/clone = UI-M-BH0-alw-f-09-0-UI/ | 118.4 | P | 32.7 | P | 98.9 | P | 271.7 |
| | | clone_end = 3/gb = AI852548/gi = 5496454/ug = Mm.1828/len = 350/STRA = for | | | | | | | |
| 6 | 103905_at | Cluster Incl AI314958 uj34107.x1 Mus musculus cDNA, 3 end/clone = IMAGE-1921861/clone_end = 3/gb = AI314958/ | 35.8 | P | 196.3 | P | 200.9 | P | 153.5 |
| | | gi = 4030225/ug = Mm.21397/len = 487/STRA = rev | | | | | | | |
| 6 | 104154_at | Cluster Incl AB021961 Mus musculus mutant p53 mRNA, complete cds/cds = (100,1272)/gb = AB021961/gi = 5421849/ | 317.7 | P | 404.3 | P | 371.5 | P | 400.6 |
| | | ug = Mm 222/len = 1429/STRA = for | | | | | | | |
| 6 | 104761_at | Cluster Incl AA612450: vo70h07 r1 Mus musculus cDNA, 5 end/clone = IMAGE-1064509/clone_end = 5/gb = AA612450/ | 194.9 | P | 217.6 | P | 256.3 | P | 259.4 |
| | | gi = 2461128/ug = Mm.24842/len = 294/STRA = for | | | | | | | |
| 6 | 94390_at | Cluster Incl AI646981 ub68c08 x1 Mus musculus cDNA, 3 end/clone = IMAGE-1382894/clone_end = 3/gb = AI646981/ | 186.4 | P | 177.7 | P | 193.9 | P | 183.1 |
| | | gi = 4725456/ug = Mm.25645/len = 540/STRA = for | | | | | | | |
| 6 | 94931_at | Cluster Incl AI851104 UI-M-BH0-akg-c-11-0-UI s1 Mus musculus cDNA, 3 end/clone = UI-M-BH0-akg-c-11-0-UI/ | 327.1 | P | 296.1 | P | 243.3 | P | 319.8 |
| | | clone_end = 3/gb = AI851104/gi = 5495010/ug = Mm 26697/len = 378/STRA = for | | | | | | | |
| 6 | 95592_at | Cluster Incl AW046785 UI-M-BH1-alz-a-05-0-UI s1 Mus musculus cDNA, 3 end/clone = UI-M-BH1-alz-a-05-0-UI/ | 319.1 | P | 92.6 | P | 306.1 | P | 296.6 |
| | | clone_end = 3/gb = AW046785/gi = 5907314/ug = Mm 279/len = 454/STRA = for | | | | | | | |
| 6 | 96812_at | Cluster Incl AF089721: Smoothened homolog (Drosophila)/cds = UNKNOWN/gb = AF089721/gi = 3608491/ug = Mm 29279/ | 116.1 | P | 119.9 | P | 129.2 | P | 189 |
| | | len = 1073/STRA = for | | | | | | | |
| 6 | 99019_at | Cluster Incl D17571. P450 (cytochrome) oxidoreductase/cds = (52,2088)/gb = D17571/gi = 407342/ug = Mm 3863/len = 2457/ | 372.1 | P | 355.5 | P | 445.4 | P | 456.8 |
| | | STRA = rev | | | | | | | |
| 6 | 103441 _at | Cluster Incl AI194248: ui14a03.r1 Mus musculus cDNA, 5 end/clone = IMAGE-1851244/clone_end = 5/gb = AI194248/ | 138 | P | 116.1 | P | 173.1 | P | 222.6 |
| | | gi = 3745455/ug = Mm.21431/len = 438/STRA = for | | | | | | | |
| 6 | 92481_at | Cluster Incl AF086905: Mus musculus protein kinase Chk2 (Chk2) mRNA, complete cds/cds = (0,1640)/gb = AF086905/ | 139.6 | P | 181.4 | P | 107.6 | P | 146.9 |
| | | gi = 3982841/ug = Mm 42149/len = 1821/STRA = for | | | | | | | |
| 6 | 97197_r_at | Cluster Incl C78850 C78850 Mus musculus cDNA, 3 end/clone = J0056C12/clone_end = 3/gb = C78850/gi = 2519180/ | 252.9 | P | 464.1 | P | 479.6 | P | 370.8 |
| | | ug = Mm 36896/len = 219/STRA = rev | | | | | | | |
| 6 | 99378_f_at | Cluster Incl M18837: Mouse MHC class I Q4 beta-2-microglobulin (Qb-1) gene, complete cds/cds = (0,980)/gb = M18837/ | 598.4 | P | 336.9 | P | 391 | P | 535.5 |
| | | gi = 199323/ug = Mm 56926/len = 981/STRA = for | | | | | | | |
| 6 | 101816_at | Cluster Incl AF063937 Squamous cell carcinoma antigen 2/cds = (72,1238)/gb = AF063937/gi = 3885487/ug = Mm 57172/ | 272.9 | P | 300.7 | P | 327.4 | P | 534.5 |
| | | len = 1239/STRA = for | | | | | | | |
| 6 | 102161_f_at | Cluster Incl X58609 Mouse MHC (Qa) Q2-k gene for class I antigen, exons 1-3/cds = (0,1088)/gb = X58609/gi = 53854/ | 725.8 | P | 373.8 | P | 371.5 | P | 665.8 |
| | | ug = Mm 46812/len = 1089/STRA = for | | | | | | | |
| 6 | 93017_at | Cluster Incl AF077527 Mus musculus syntenin mRNA, complete cds/cds = (54,950)/gb = AF077527/gi = 3342559/ | 68.5 | P | 71.6 | P | 86 | P | 86.4 |
| | | ug = Mm 14744/len = 2043/STRA = for | | | | | | | |
| 6 | 93590_at | Cluster Incl AI844370 UI-M-AL1-ahp-b-12-0-UI s1 Mus musculus cDNA, 3 end/clone = UI-M-AL1-ahp-b-12-0-UI/ | 130.8 | P | 160.8 | A | 181.8 | P | 176.6 |
| | | clone_end = 3/gb = AI844370/gi = 5488276/ug = Mm 20818/len = 430/STRA = for | | | | | | | |
| 6 | 93775_at | Cluster Incl AI841894 UI-M-AO0-acd-d-09-0-UI s1 Mus musculus cDNA, 3 end/clone = UI-M-AO0-acd-d-09-0-UI/ | 307.3 | P | 345.2 | P | 343.1 | P | 261.6 |
| | | clone_end = 3/gb = AI841894/gi = 5476107/ug = Mm.2121/len = 435/STRA = for | | | | | | | |
| 6 | 94298_at | Cluster Incl X53619 M musculus P35B gene, exon 1/cds = (0,815)/gb = X53619/gi = 53556/ug = Mm 22596/len = 816/ | 165.8 | A | 233 | P | 325.3 | P | 334.4 |
| | | STRA = for | | | | | | | |
| 6 | 97876_at | Cluster Incl AI842008 UI-M-AH0-ada-c-09-0-UI.s1 Mus musculus cDNA, 3 end/clone = UI-M-AH0-ada-c-09-0-UI/ | 550.8 | P | 489.6 | P | 402.1 | P | 560.9 |
| | | clone_end = 3/gb = AI842008/gi = 5476221/ug = Mm.28356/len = 431/STRA = for | | | | | | | |
| 6 | 97515_at | Cluster Incl X89998 M musculus mRNA for 17 beta-hydroxysteroid dehydrogenase type IV/cds = (12,2219)/gb = X89998/ | 167.6 | P | 235.9 | P | 342.5 | P | 283.8 |
| | | gi = 1213007/ug = Mm 3195/len = 2468/STRA = for | | | | | | | |
| 6 | 97541_f_at | Cluster Incl X00246 Mouse mRNA with a Set 1 repetitive element for a class I major histocompatibility complex(MHC) | 199.6 | P | 45.4 | P | 36.5 | P | 239.3 |
| | | antigen/cds = (0,857)/gb = X00246/gi = 53085/ug = Mm.33263/len = 1484/STRA = rev | | | | | | | |
| 6 | 98088_at | Cluster Incl X13333 CD14 antigen/cds = (32,1132)/gb = X13333/gi = 50338/ug = Mm.3460/len = 1366/STRA = for | 663 | P | 691.8 | P | 1477.4 | P | 2424.1 |
| 6 | 98092_at | Cluster Incl AA790307 vw17a10 r1 Mus musculus cDNA, 5 end/clone = IMAGE-1244058/clone_end = 5/gb = AA790307/ | 410.5 | P | 301.1 | P | 62.8 | P | 894.2 |
| | | gi = 2850427/ug = Mm.34609/len = 581/STRA = for | | | | | | | |
| 6 | 98589_at | Cluster Incl M93275 Adipose differentiation related protein/cds = (78,1335)/gb = M93275/gi = 191692/ug = Mm 381/len = 1680/ | 1344.4 | P | 1052.1 | A | 1297.6 | P | 1745.2 |
| | | STRA = for | | | | | | | |
| 6 | 101540_at | Cluster Incl AF069519: Mus musculus T-G mismatch-specific thymine-DNA glycosylase TDGb isoform mRNA, complete | 495.8 | P | 345.4 | P | 536.3 | P | 563.7 |
| | | cds/cds = (108,1301)/gb = AF069519/gi = 3420292/ug = Mm 88099/len = 2859/STRA = for | | | | | | | |
| 6 | 101566_f_at | Cluster Incl M17818 Mouse major urinary prolein group 1 gene, 5 end, clone BL6-3/cds = (66,95)/gb = M17818/gi = 202305/ | 529.2 | P | 687.7 | P | 2074.5 | P | 447.6 |
| | | ug = Mm 89481/len = 96/STRA = for | | | | | | | |
| 6 | 103808_at | Cluster Incl AB016784 Mus musculus ref-11 mRNA, complete cds/cds = (261,2450)/gb = AB016784/gi = 3551508/ | 276 | P | 247.9 | P | 394.2 | P | 244.1 |
| | | ug = Mm 20885/len = 2829/STRA = for | | | | | | | |
| 6 | 98410_at | Cluster Incl AJ007972 Mus musculus mRNA for GTPI protein/cds = (16,1203)/gb = AJ1007972)/gi = 4158175/ug = Mm 33902/ | 312 | P | 198.9 | P | 162.4 | P | 289.8 |
| | | len = 3338/STRA = for | | | | | | | |
| 6 | 101465_at | Cluster Incl U06924 Signal transducer and activator of transcription 1/cds = (4,2253)/gb = U06924/gi = 458709/ug = Mm 8249/ | 91.8 | P | 35.2 | P | 37.9 | P | 137.9 |
| | | len = 2277/STRA = for | | | | | | | |
| 6 | 101923_at | Cluster Incl U34277 Mus musculus PAF acetylhydrolase mRNA, complete cds/cds = (116,1438)/gb = U34277/gi = 1041235/ | 248.9 | P | 212.5 | A | 229.3 | P | 410.6 |
| | | ug = Mm 9277/len = 1507/STRA = for | | | | | | | |
| 6 | 102641_at | Cluster Incl L03215: SFFV proviral integration 1/cds = UNKNOWN/gb = L03215/gi = 201035/ug = Mm.1302/len = 1232/ | 239.9 | A | 273.7 | P | 291 | P | 353 |
| | | STRA = for | | | | | | | |
| 6 | 102706_l_at | Cluster Incl X61597 M musculus gene for kallikrein-binding protein/cds = (111,1364)/gb = X61597/gi = 52808/ug = Mm 14191/ | 36.5 | P | 83.4 | P | 52.8 | P | 119.8 |
| | | len = 1690/STRA = for | | | | | | | |
| 6 | 102707_f_at | Cluster Incl X61597 M musculus gene for kallikrein-binding protein/cds = (111,1364)/gb = X61597/gi = 52808/ug = Mm 14191/ | 68.5 | M | 178.1 | P | 242.5 | P | 475.2 |
| | | len = 1690/STRA = for | | | | | | | |
| 6 | 104202_at | Cluster Incl AI850624 UI-M-BG1-alk-d-02-0-UI s1 Mus musculus cDNA, 3 end/clone = UI-M-BG1-alk-d-02-0-UI/ | 190.1 | P | 259.7 | P | 361.2 | P | 340.6 |
| | | clone_end = 3/gb = AI850624/gi = 5494530/ug = Mm 27539/len = 416/STRA = rev | | | | | | | |
| 6 | 104456_at | Cluster Incl AA863742 vx08b05 r1 Mus musculus cDNA, 5 end/clone = IMAGE-1263825/clone_end = 5/gb = AA863742/ | 108.6 | P | 65.7 | P | 85.2 | P | 118.6 |
| | | gi = 2956221/ug = Mm 29514/len = 402/STRA = for | | | | | | | |
| 6 | 104677_at | Cluster Incl AI852008: UI-M-BH0-aiq-f-08-0-UI.s1 Mus musculus cDNA, 3 end/clone = UI-M-BH0-aiq-f-08-0-UI/ | 108.8 | P | 138.9 | P | 157.3 | P | 149.4 |
| | | clone_end = 3/gb = AI852008/gi = 5495914/ug = Mm 32542/len = 410/STRA = for | | | | | | | |
| 6 | 92251_f_at | Cluster Incl AA960657 ub60c10 s1 Mus musculus cDNA, 3 end/clone = IMAGE-1382130/clone_end = 3/gb = AA960657/ | 167.3 | P | 124.3 | P | 170.1 | P | 304.5 |
| | | gi = 3126557/ug = Mm 35110/len = 467/STRA = rev | | | | | | | |
| 6 | 93339_at | Cluster Incl AI846243 UI-M-AK1-aez-a-10-0-UI s1 Mus musculus cDNA, 3 end/clone = UI-M-AK1-aez-a-10-0-UI/ | 395.2 | P | 380.6 | P | 305.3 | P | 455.9 |
| | | clone_end = 3/gb = AI846243/gi = 5490149/ug = Mm 18706/len = 334/STRA = for | | | | | | | |
| 6 | 99663_g_at | Cluster Incl AI194767: ul56d10 x1 Mus musculus cDNA, 3 end/clone = IMAGE-1886419/clone_end = 3/gb = AI194767/ | 81.5 | P | 286.5 | P | 262.7 | P | 337.4 |
| | | gi = 3747373/ug = Mm 4379/len = 525/STRA = for | | | | | | | |
| 6 | 101588_at | Cluster Incl AF058055: Mus musculus monocarboxylate transpoter 1 (mMCT1) mRNA, complete cds/cds = (189,1670)/ | 4 | A | 63 | P | 28.5 | P | 94.5 |
| | | gb = AF058055/gi = 3047399/ug = Mm 9086/len = 3184/STRA = for | | | | | | | |
| 6 | 102027_s_at | Cluster Incl AA204010 mu59g02 r1 Mus musculus cDNA, 5 end/clone = IMAGE-643730/clone_end = 5/gb = AA204010/ | 103.7 | P | 112.6 | P | 145.4 | A | 142.3 |
| | | gi = 1800701/ug = Mm 10124/len = 304/STRA = for | | | | | | | |
| 6 | 103016_s_at | Cluster Incl X68273: CD68 antigen/cds = (91,1071)/gb = X68273/gi = 52988/ug = Mm 15819/len = 1234/STRA = for | 223.1 | P | 232.8 | P | 227.9 | P | 250.4 |
| 6 | 103397_at | Cluster Incl AA795486 vq95e08 r1 Mus musculus cDNA, 5 end/clone = IMAGE-1110086/clone_end = 5/gb = AA795486/ | 113.4 | P | 72.2 | A | 104.7 | P | 82.7 |
| | | gi = 2858441/ug = Mm 18713/len = 273/STRA = for | | | | | | | |
| 6 | 95543_at | Cluster Incl AI843046 UI-M-AK1-aeq-g-04-0-UI s1 Mus musculus cDNA, 3 end/clone = UI-M-AK1-aeq-g-04-0-UI/ | 439 | P | 266.9 | P | 351.6 | P | 501.5 |
| | | clone_end = 3/gb = AI843046/gi = 5477259/ug = Mm 27685/len = 432/STRA = rev | | | | | | | |
| 6 | 97346_at | Cluster Incl AW124115: UI-M-BH2.1-apr-b-11-0-UI s1 Mus musculus cDNA, 3 end/clone = UI-M-BH2.1-apr-b-11-0-UI/ | 167 | P | 121.4 | P | 257.1 | P | 205.9 |
| | | clone_end = 3/gb = AW124115/gi = $$/ug = Mm $$/len = $$/STRA = for | | | | | | | |
| 6 | 98428_at | Cluster Incl AJ246002: Mus musculus mRNA for spastin protein orthologue (Spast gene)/cds = (0,1516)/gb = AJ246002/ | −5.3 | P | 45.8 | P | 26.5 | P | 49.9 |
| | | gi = 6273571/ug = Mm 34234/len = 1689/STRA = for | | | | | | | |
| 6 | 101160_at | Cluster Incl X53798: Macrophage inflammatory protein 2/cds = (46,348)/gb = X53798/gi = 53128/ug = Mm 4979/len = 1083/ | 45.6 | A | 75.9 | P | 537.4 | P | 699.8 |
| | | STRA = for | | | | | | | |
| 6 | 96031_r_at | Cluster Incl AW259411. up29c03.x1 Mus musculus cDNA, 3 end/clone = IMAGE-2655748/clone_end = 3/gb = AW259411/ | 106.9 | P | 84.6 | P | 50.2 | P | 85.5 |
| | | gi = 6632465/ug = Mm 25794/len = 465/STRA = for | | | | | | | |
| 6 | 95752_at | Cluster Incl AI837369.UI-M-AP0-abm-a-02-0-UI s1 Mus musculus cDNA, 3 end/clone = UI-M-AP0-abm-a-02-0-UI/ | 310.3 | P | 144.4 | M | 353.8 | P | 384.5 |
| | | clone_end = 3/gb = AI837369/gi = 5471582/ug = Mm 29784/len = 371/STRA = for | | | | | | | |
| 6 | 97507_at | Cluster Incl X67809 Peptidylprotyl isomerase C-associated protein/cds = (168,1901)/gb = X67809/gi = 297032/ug = Mm 3152/ | 109.1 | P | 28.4 | A | 30.7 | P | 131.6 |
| | | len = 2168/STRA = for | | | | | | | |
| 6 | 102313_at | Cluster Incl L09737 GTP cyclohydrolase 1/cds = (122,847)/gb = L09737/gi = 293356/ug = Mm. 10651/len = 994/STRA = for | 102.7 | P | 83.2 | A | 102.2 | P | 166.5 |
| 6 | 103805_g_at | Cluster Incl AW231026: uo70a01.y1 Mus musculus cDNA, 5 end/clone = IMAGE-2647848/clone_end = 5/gb = AW231026/ | 146.8 | P | 162.5 | P | 131.6 | A | 219.4 |
| | | gi = 6580258/ug = Mm 20156/len = 482/STRA = for | | | | | | | |
| 6 | 103887_at | Cluster Incl M83219 Mus musculus intracellular calcium-binding protein (MRP14) mRNA, complete cds/cds = (33,374)/ | −2.3 | A | 14.4 | P | 1008.5 | P | 3557 |
| | | gb = M83219/gi = 199807/ug = Mm 2128/len = 488/STRA = for | | | | | | | |
| 6 | 104528_at | Cluster Incl AI181549: uc59h04 r1 Mus musculus cDNA, 5 end/clone = IMAGE-1429975/clone_end = 5/gb = AI181549/ | 106.4 | P | 20.2 | P | 120.8 | M | 96.1 |
| | | gi = 3732187/ug = Mm 2310/len = 435/STRA = for | | | | | | | |
| 6 | 104549_at | Cluster Incl AI844761: UI-M-AL1-ahq-e-12-0-UI s1 Mus musculus cDNA, 3 end/clone = UI-M-AL1-ahq-e-12-0-UI/ | 208.4 | P | 155 | A | 293.2 | P | 259.2 |
| | | clone_end = 3/gb = AI844761/gi = 5488667/ug = Mm 23702/len = 384/STRA = for | | | | | | | |
| 6 | 104606_at | Cluster Incl M55561 CD80 antigen/cds = (78,302)/gb = M55561/gi = 200225/ug = Mm 24130/len = 491/STRA = for | 527.9 | P | 359.8 | P | 496.5 | P | 669 |
| 6 | 96188_at | Cluster Incl AF052506 Mus musculus double-stranded RNA-specific adenosine deaminase mRNA, complete cds/ | 131.7 | P | 130.7 | A | 111 | P | 189 |
| | | cds = (6,3484)/gb = AF052506/gi = 2981096/ug = Mm 28502/len = 3466/STRA = for | | | | | | | |
| 6 | 97409_at | Cluster Incl U19119 Mus musculus G-protein-like LRG-47 mRNA, complete cds/cds = (445,1674)/gb = U19119/gi = 633753/ | 121.1 | P | 34.7 | P | 21 | A | 122.2 |
| | | ug = Mm 29938/len = 2227/STRA = for | | | | | | | |
| 6 | 98500_at | Cluster Incl D13695 Lymphocyte antigen 84/cds = (42,1745)/gb = D13695/gi = 286100/ug = Mm 35692/len = 4989/STRA = for | 73.5 | P | 70.3 | P | 123 | P | 141.1 |
| 6 | 103295_at | Cluster Incl AI854482 UI-M-BH0-ait-h-07-0-UI s1 Mus musculus cDNA, 3 end/clone = UI-M-BH0-ait-h-07-0-UI/clone_end = 3/ | 269.8 | A | 244 | P | 341.8 | A | 247 |
| | | gb = AI854482/gi = 5498388/ug = Mm 20971/len = 389/STRA = for | | | | | | | |
| 6 | 101681_f_at | Cluster Incl U21906 Histocompatibility 2, blastocyst/cds = (236,1285)/gb = U21906/gi = 2326165/ug = Mm 34289/len = 1515/ | 569.8 | A | 375.4 | A | 572 | P | 801.9 |
| | | STRA = for | | | | | | | |
| 6 | 92811_at | Cluster Incl X74154 Retinol binding protein 2, cellular/cds = (57,461)/gb = X74154/gi = 397351/ug = Mm 12825/len = 479/ | 46.9 | A | 88.8 | A | 96.8 | P | 101 |
| | | STRA = for | | | | | | | |
| 6 | 95505_at | Cluster Incl AW060509 UI-M-BH1-ann-c-12-0-UI.s1 Mus musculus cDNA, 3 end/clone = UI-M-BH1-ann-c-12-0-UI/ | 17.5 | P | 4.2 | M | 18.5 | M | 90.4 |
| | | clone_end = 3/gb = AW060509/gi = 6008260/ug = Mm.25187/len = 308/STRA = for | | | | | | | |
| 6 | 98045_s_at | Cluster Incl U18869: Mus musculus mitogen-responsive 96 kDa phosphoprotein p96 mRNA, alternatively spliced p67 | 252.7 | P | 328.7 | P | 328.2 | P | 508.2 |
| | | mRNA, and alternatively spliced p93 mRNA, complete cds/cds = (216,2516)/gb = U18869/gi = 1176369/ug = Mm 34248/ | | | | | | | |
| | | len = 3347/STRA = for | | | | | | | |
| 6 | 98562_at | Cluster Incl X58861: Complement component 1, q subcomponent, alpha polypeptide/cds = (29,766)/gb = X58861/gi = 50226/ | 371.8 | P | 415.3 | P | 483.8 | P | 309.4 |
| | | ug = Mm.370/len = 1033/STRA = for | | | | | | | |
| 6 | 100611_at | Cluster Incl M21050 Mouse lysozyme M gene/cds = (30,476)/gb = M21050/gi = 198950/ug = Mm 654/len = 1264/STRA = for | 156.7 | P | 75.4 | P | 263.4 | P | 192.7 |
| 6 | 102351_at | Cluster Incl J05118: Carboxypeptidase A3, mast cell/cds = (6,1259)/gb = J05118/gi = 192372/ug = Mm 1135/len = 1442/ | 264.4 | P | 342.7 | P | 1108.2 | P | 537.1 |
| | | STRA = rev | | | | | | | |
| 6 | 102791_at | Cluster Incl U22033 Large multifunctional protease 7/cds = (0,830)/gb = U22033/gi = 972985/ug = Mm 13913/len = 831/ | 243 | P | 219.4 | A | 206.4 | A | 313.5 |
| | | STRA = for | | | | | | | |
| 6 | 104728_at | Cluster Incl L27439 Mus musculus protein S mRNA, partial cds/cds = (0,1933)/gb = L27439/gi = 487866/ug = Mm 24611/ | −24.4 | A | 43.7 | A | 96.1 | P | 139.9 |
| | | len = 3079/STRA = for | | | | | | | |
| 6 | 98465_f_at | Cluster Incl M31419 Interferon activated gene 204/cds = (127,2049)/gb = M31419/gi = 198399/ug = Mm 34817/len = 2353/ | 120.6 | P | 17.5 | A | 28.9 | A | 413.4 |
| | | STRA = for | | | | | | | |
| 6 | 100397_at | Cluster Incl AF024637 TYRO protein tyrosine kinase binding protein/cds = (0,344)/gb = AF024637/gi = 2906001/ | 274.2 | P | 338.2 | P | 518.5 | P | 510 |
| | | ug = Mm 46301/len = 345/STRA = for | | | | | | | |
| 6 | 102264_at | Cluster Incl AF099972 Mus musculus schlafen1 (Slfn1) mRNA, complete cds/cds = (257,1270)/gb = AF099972/gi = 3983147/ | 243.4 | P | 92.6 | A | 213.7 | P | 271.4 |
| | | ug = Mm 10948/len = 1881/STRA = for | | | | | | | |
| 6 | 103210_at | Cluster Incl M29855 Interleukin 3 receptor, beta chain 2/cds = (175,2811)/gb = M29855/gi = 198342/ug = Mm 1940/len = 4613/ | 115.2 | P | 34.2 | M | 128 | A | 160 |
| | | STRA = for | | | | | | | |
| 6 | 103226_at | Cluster Incl Z11974 Mannose receptor, C type 1/cds = (99,4469)/gb = Z11974/gi = 52997/ug = Mm 2019/len = 5085/ | 132.7 | P | 241.5 | P | 235.6 | P | 155.1 |
| | | STRA = for | | | | | | | |
| 6 | 103707_at | Cluster Incl U77461 Complement component 3a receptor 1/cds = (0,1433)/gb = U77461/gi = 2130534/ug = Mm 2408/ | 174.7 | P | 166.1 | P | 210.3 | P | 283.1 |
| | | len = 1434/STRA = for | | | | | | | |
| 6 | 92223_at | Cluster Incl X66295: Complement component 1, q subcomponent, c polypeptide/cds = (38,778)/gb = X66295/gi = 50228/ | 266.7 | P | 648.1 | P | 753.5 | P | 462.5 |
| | | ug = Mm.3453/len = 1019/STRA = for | | | | | | | |
| 6 | 93622_at | Cluster Incl M57626 Mast cell protease 6/cds = (33,863)/gb = M57626/gi = 200508/ug = Mm.7409/len = 1108/STRA = for | 1190.4 | P | 1721.6 | P | 2169.7 | P | 1676.5 |
| 6 | 99413_at | Cluster Incl U29678 Chemokine (C-C) receptor 1/cds = (0,1067)/gb = U29678/gi = 1109785/ug = Mm 57051/len = 1068/ | 24.7 | P | 54.9 | A | 74 | P | 236.1 |
| | | STRA = for | | | | | | | |
| 6 | 100328_s_at | Cluster Incl U96684 Paired-lg-like receptor A3/cds = (10,2001)/gb = U96684/gi = 2138356/ug = Mm 87801/len = 2101/ | 224.7 | P | 129 | P | 185.2 | P | 316 |
| | | STRA = for | | | | | | | |
| 6 | 95806_f_at | Cluster Incl M68899 Mouse mast cell chymase 2 mRNA, complete cds/cds = (120,650)/gb = M68899/gi = 199030/ | 215.5 | P | 338.6 | P | 460.6 | P | 408.5 |
| | | ug = Mm 87865/len = 901/STRA = for | | | | | | | |
| 6 | 102104_f_at | Cluster Incl AI504305 vl04g11 x1 Mus musculus cDNA, 3 end/clone = IMAGE-963236/clone_end = 3/gb = AI504305/ | 187.2 | P | 151.9 | P | 171.7 | P | 283.3 |
| | | gi = 4402156/ug = Mm.42485/len = 580/STRA = rev | | | | | | | |
| 6 | 95159_at | Cluster Incl AI846849: UI-M-AI1-afk-e-04-0-UI.s1 Mus musculus cDNA, 3 end/clone = UI-M-AI1-afk-o-04-0-UI/clone_end = 3/ | 123.8 | P | 157.5 | A | 73.7 | A | 166.4 |
| | | gb = AI846849/gi = 5490755/ug = Mm.29484/len = 332/STRA = for | | | | | | | |
| 6 | 96297_at | Cluster Incl AI845934: UI-M-AK1-aex-f-03-0-UI.s1 Mus musculus cDNA, 3 end/clone = UI-M-AK1-aex-f-03-0-UI/ | 97.4 | P | 61.9 | P | 12.5 | A | 145.1 |
| | | clone_end = 3/gb = AI845934/gi = 5489840/ug = Mm.29906/len = 280/STRA = for | | | | | | | |
| 6 | 97530_at | Cluster Incl U82627: Mus musculus ubiquitin-conjugating enzyme mE2 mRNA, complete cds/cds = (105,581)/gb = U82627/ | 137.9 | P | 87.2 | A | 113 | A | 120.7 |
| | | gi = 1857450/ug = Mm 3268/len = 769/STRA = for | | | | | | | |
| 6 | 100100_at | Cluster Incl AA762212-vv47h06 r1 Mus musculus cDNA, 5 end/clone = IMAGE-1225595/clone_end = 5/gb = AA762212/ | 38.5 | A | 46.6 | P | 76 | P | 59.6 |
| | | gi = 2811959/ug = Mm 46316/len = 509/STRA = for | | | | | | | |
| 6 | 102021_at | Cluster Incl M27960 Mouse interleukin-4 receptor (secreted form) mRNA, complete cds/cds = (236,928)/gb = M27960/ | 11.5 | A | −22.2 | A | 238.5 | P | 458 |
| | | gi = 198365/ug = Mm 1009/len = 3697/STRA = for | | | | | | | |
| 6 | 102353_at | Cluster Incl M31039 Mus musculus complement receptor 3 beta subunit MAC-1/mRNA, complete cds/cds = (0,2315)/ | 29.1 | P | −14 | A | 23.5 | P | 98.8 |
| | | gb = M31039/gi = 198434/ug = Mm 1137/len = 2459/STRA = for | | | | | | | |
| 6 | 103394_at | Cluster Incl U72680 Mus musculus ion channel homolog RIC mRNA, complete cds/cds = (48,584)/gb = U72680/gi = 1872490/ | 108.4 | A | 153.9 | P | 186.4 | P | 426.6 |
| | | ug = Mm 1870/len = 797/STRA = for | | | | | | | |
| 6 | 96772_at | Cluster Incl J04620 Mouse primase p49 subunit (prlA) mRNA, complete cds/cds = (73,1326)/gb = J04620/gi = 200196/ | 199.8 | P | 211.9 | P | 201.3 | P | 188 |
| | | ug = Mm 2903/len = 1507/STRA = for | | | | | | | |
| 6 | 99979_at | Cluster Incl X78445 M musculus Cyp1-b-1 mRNA for cytochrome P450/cds = (179,1810)/gb = X78445/gi = 506463/ | −68.3 | A | −72.6 | A | 20.2 | A | 85.4 |
| | | ug = Mm 4443/len = 2006/STRA = for | | | | | | | |
| 6 | 101392_at | Cluster Incl AW122839 UI-M-BH2 1-aoz-e-09-0-UI.s1 Mus musculus cDNA, 3 end/clone = UI-M-BH2.1-aoz-e-09-0-UI/ | 77.6 | A | 138.1 | A | 109.7 | P | 157.2 |
| | | clone_end = 3/gb = AW122839/gi = 6098369/ug = Mm 7196/len = 382/STRA = for | | | | | | | |
| 6 | 102292_at | Cluster Incl U00937: Mus musculus GADD45 protein (gadd45) gene, complete cds/cds = (0,497)/gb = U00937/gi = 392933/ | 74.3 | P | −168.7 | A | 84.1 | P | 165.4 |
| | | ug = Mm 1236/len = 498/STRA = for | | | | | | | |
| 6 | 104006_at | Cluster Incl L21768 Mus musculus eps15 mRNA, complete cds/cds = (110,2803)/gb = L21768/gi = 404756/ug = Mm 267/ | 103.7 | A | 123.5 | A | 99.8 | P | 172.7 |
| | | len = 3033/STRA = for | | | | | | | |
| 6 | 104657_at | Cluster Incl D26177.Leukemia Inhibitory factor receptor/cds = (71,3349)/gb = D26177/gi = 473718/ug = Mm.3174/len = 3399/ | 18.4 | A | −11 | A | 29.7 | P | 66.2 |
| | | STRA = for | | | | | | | |
| 6 | 92443_I_at | Cluster Incl X16493 Zinc finger protein 1/cds = (320,1594)/gb = X16493/gi = 55482/ug = Mm 4184/len = 1939/STRA = rev | 27 | P | −0.9 | A | 48.7 | A | 90.1 |
| 6 | 92472_f_at | Cluster Incl AF099973 Mus musculus schlafen2 (Slfn2) mRNA, complete cds/cds = (182,1318)/gb = AF099973/gi = 3983149/ | 76.7 | M | 78.8 | P | 113.4 | P | 227 |
| | | ug = Mm 42124/len = 1684/STRA = for | | | | | | | |
| 6 | 92718_at | Cluster Incl AI158810 ud38d09 r1 Mus musculus cDNA, 5 end/clone = IMAGE-1448177/clone_end = 5/gb = AI158810/ | 348.2 | P | 227.9 | P | 130.1 | A | 298.1 |
| | | gi = 3691992/ug = Mm 48382/len = 251/STRA = for | | | | | | | |
| 6 | 94774_at | Cluster Incl M31416 Interferon activated gene 202/cds = (293,1830)/gb = M31418/gi = 198396/ug = Mm.16744/len = 1857/ | −4.9 | A | −10.8 | A | −10.9 | A | 277.6 |
| | | STRA = for | | | | | | | |
| 6 | 98822_at | Cluster Incl X56602 Mus musculus mRNA Interferon-induced 15-KDa protein/cds = (111,593)/gb = X58602/gi = 52737/ | 88 | A | −27.6 | A | −52.5 | A | 849.9 |
| | | ug = Mm 4950/len = 728/STRA = for | | | | | | | |
| 6 | 97013_f_at | Cluster Incl AW046124 UI-M-BH1-alf-e-03-0-UI s1 Mus musculus cDNA, 3 end/clone = UI-M-BH1-alf-e-03-0-Ul/ | 190.3 | A | 307.7 | P | 341.8 | P | 387.8 |
| | | clone_end = 3/gb = AW046124/gi = 5906653/ug = Mm.88950/len = 249/STRA = rev | | | | | | | |
| 6 | 101635_f_at | Cluster Incl M16360 Mouse major unnary protein V (MUP V) mRNA, complete cds/cds = (58,600)/gb = M16360/gi = 199884/ | 82.3 | A | 248.1 | P | 498.8 | P | 226 |
| | | ug = Mm 30274/len = 872/STRA = for | | | | | | | |
| 6 | 101682_f_at | Cluster Incl M16358 Major unnary protein 4/cds = (66,602)/gb = M16358/gi = 199882/ug = Mm 34335/len = 875/STRA = for | 71.2 | P | 113.9 | P | 426.7 | P | 77.2 |
| 7 | 92539_at | Cluster Incl M16465 Calpactin I light chain/cds = (68,361)/gb = M16465/gi = 192360/ug = Mm.1/len = 600/STRA = for | 3206.3 | P | 4060.2 | P | 4087.8 | P | 4408.3 |
| 7 | 92603_at | Cluster Incl U13840: Mus musculus vacuolar adenosine triphosphatase subunit D mRNA, complete cds/cds = (17,1072)/ | 1015.9 | P | 931.2 | P | 1233.5 | P | 888 |
| | | gb = U13840/gi = 3955099/ug = Mm 1081/len = 1509/STRA = for | | | | | | | |
| 7 | 92607_at | Cluster Incl AF017994.Mus musculus Peg1/MEST protein (Peg1/MEST) gene, partial cds/cds = (247,427)/gb = AF017994/ | 924.7 | P | 1601.6 | P | 1941.2 | P | 1286.2 |
| | | gi = 2570405/ug = Mm.1089/len = 428/STRA = for | | | | | | | |
| 7 | 92638_at | Cluster Incl Z67745 M. musculus mRNA for phosphatase 2A catalytic subunit, Isotype alpha/cds = (0,929)/gb = Z67745/ | 317.5 | P | 525.5 | P | 851.1 | P | 531.2 |
| | | gi = 1246794/ug = Mm.11711/len = 930/STRA = for | | | | | | | |
| 7 | 92855_at | Cluster Incl Z50159 M musculus mRNA for Sui1/cds = (0,230)/gb = Z50159/gi = 927434/ug = Mm 13886/len = 355/STRA = for | 1263.6 | P | 1344.6 | P | 2280.4 | P | 1444.6 |
| 7 | 93093_at | Cluster Incl U35623.Myeloid cell leukemia sequence 1/cds = (48,1043)/gb = U35623/gi = 2522268/ug = Mm 1639/len = 3396/ | 971.2 | P | 1070.3 | P | 1208.7 | P | 1108.4 |
| | | STRA = for | | | | | | | |
| 7 | 93100_at | Cluster Incl X13297 Mouse mRNA for vascular smooth muscle alpha-actin/cds = (49,1182)/gb = X13297/gi = 49861/ | 1028.9 | P | 1265.3 | P | 1227.1 | P | 899.8 |
| | | ug = Mm 16537/len = 1340/STRA = for | | | | | | | |
| 7 | 93327_at | Cluster Incl AI842665 UI-M-AO1-aeo-a-02-0-UI.s1 Mus musculus cDNA, 3 end/clone = UI-M-AO1-aeo-a-02-0-UI/ | 225.5 | P | 262.7 | P | 281.9 | P | 168.1 |
| | | clone_end = 3/gb = AI842685/gi = 5476878/ug = Mm 186/len = 288/STRA = for | | | | | | | |
| 7 | 93353_at | Cluster Incl AF013262: Lumican/cds = (109,1125)/gb = AF013262/gi = 2654773/ug = Mm 18888/len = 1782/STRA = for | 2016 | P | 2370 | P | 3180.7 | P | 1167.3 |
| 7 | 93517_at | Cluster Incl Z18272 Procollagen, type VI, alpha 2/cds = (0,1115)/gb = Z18272/gi = 57957/ug = Mm.1949/len = 1562/ | 1611.3 | P | 2974.8 | P | 3190.7 | P | 1828.3 |
| | | STRA = for | | | | | | | |
| 7 | 93743_at | Cluster Incl AI838388: UI-M-AJO-aav-f-02-0-UI.s2 Mus musculus cDNA, 3 end/clone = UI-M-AJ0-aav-f-02-0-UI/ | 172.5 | P | 219 | P | 196.4 | P | 177.4 |
| | | clone_end = 3/gb = AI838388/gi = 5472601/ug = Mm.21069/len = 414/STRA = for | | | | | | | |
| 7 | 93793_at | Cluster Incl AW122780 UI-M-BH2.1-aoy-c-12-0-UI s1 Mus musculus cDNA, 3 end/clone = UI-M-BH2 1-aoy-c-12-0-UI/ | 951.6 | P | 1096.8 | P | 1441.8 | P | 996 |
| | | clone_end = 3/gb = AW122780/gi = 6098310/ug = Mm.21387/len = 148/STRA = for | | | | | | | |
| 7 | 93975_at | Cluster Incl AI853531 UI-M-BH0-ajd-f-01-0-UI s1 Mus musculus cDNA, 3 end/clone = UI-M-BH0-ajd-f-01-0-UI/ | 227.9 | P | 244.6 | P | 1146.5 | P | 496.5 |
| | | clone_end = 3/gb = AI853531/gi = 5497437/ug = Mm 21679/len = 425/STRA = for | | | | | | | |
| 7 | 94060_at | Cluster Incl AI852623 UI-M-BH0-aiu-e-12-0-UI.s1 Mus musculus cDNA, 3 end/clone = UI-M-BH0-alu-e-12-0-UI/ | 378.1 | P | 408.7 | P | 644.7 | P | 531 |
| | | clone_end = 3/gb = AI852623/gi = 5496529/ug = Mm.22044/len = 416/STRA = for | | | | | | | |
| 7 | 94218_at | Cluster Incl AW121091.UI-M-BH2 3-aof-h-07-0-UI s1 Mus musculus cDNA, 3 end/clone = UI-M-BH2 3-aof-h-07-0-UI/ | 1509.5 | P | 1861.7 | P | 1710.5 | P | 1325.8 |
| | | clone_end = 3/gb = AW121091/gi = 6096424/ug = Mm.22227/len = 214/STRA = for | | | | | | | |
| 7 | 94238_at | Cluster Incl AW228316 up21f09 y1 Mus musculus cDNA, 5 end/clone = IMAGE-2655017/clone_end = 5/gb = AW228316/ | 158.7 | P | 186.3 | P | 309 | P | 131.7 |
| | | gi = 6557612/ug = Mm 22345/len = 193/STRA = for | | | | | | | |
| 7 | 94246_at | Cluster Incl J04103: E26 avian leukemia oncogene 2,3 domain/cds = (137,1543)/gb = J04103/gi = 193193/ug = Mm.22365/ | 648.4 | P | 645.4 | P | 1460.5 | P | 770.6 |
| | | len = 1552/STRA = for | | | | | | | |
| 7 | 94256_at | Cluster Incl AI849533 UI-M-AH1-agt-h-01-0-UI.s1 Mus musculus cDNA, 3 end/clone = UI-M-AH1-agt-h-01-0-UI/ | 1142.5 | P | 1200.3 | P | 1534.8 | P | 1428.2 |
| | | clone_end = 3/gb = AI849533/gi = 5493439/ug = Mm 22409/len = 360/STRA = for | | | | | | | |
| 7 | 94258_at | Cluster Incl L07918 Mus musculus GDP-dissociation inhibitor mRNA, preferentially expressed in hematopoietic cells, | 1146 | P | 1309.2 | P | 1286.3 | P | 1467.1 |
| | | complete cds/cds = (32,634)/gb = L07918/gi = 193461/ug = Mm 2241/len = 1099/STRA = rev | | | | | | | |
| 7 | 94301_at | Cluster Incl AI843269.UI-M-AO1-aei-c-08-0-UI s1 Mus musculus cDNA, 3 end/clone = UI-M-AO1-aei-c-08-0-UI/ | 740.4 | P | 857.1 | P | 898.3 | P | 643.8 |
| | | clone_end = 3/gb = AI843269/gi = 5477482/ug = Mm 22602/len = 310/STRA = for | | | | | | | |
| 7 | 94303_at | Cluster Incl U11274 Mus musculus clone pmuAUF1-3 RNA-binding protein AUF1 mRNA, complete cds/cds = (142,951)/ | 963.3 | P | 923.6 | P | 1204.5 | P | 817.3 |
| | | gb = U11274/gi = 508269/ug = Mm 22617/len = 1322/STRA = for | | | | | | | |
| 7 | 95405_at | Cluster Incl AW045534: UI-M-BH1-aks-c-09-0-UI s1 Mus musculus cDNA, 3 end/clone = UI-M-BH1-aks-c-09-0-UI/ | 328.7 | P | 385.2 | P | 323.7 | P | 327.5 |
| | | clone_end = 3/gb = AW045534/gi = 5906063/ug = Mm 24209/len = 435/STRA = for | | | | | | | |
| 7 | 95413_at | Cluster Incl AI851332 UI-M-BH0-akh-b-07-0-UI s1 Mus musculus cDNA, 3 end/clone = UI-M-BH0-akh-b-07-0-UI/ | 862.1 | P | 958.3 | P | 1163.6 | P | 1137.6 |
| | | clone_end = 3/gb = AI851332/gi = 5495238/ug = Mm 24272/len = 387/STRA = for | | | | | | | |
| 7 | 95477_at | Cluster Incl AW125185 UI-M-BH2 1-apz-g-10-0-UI s1 Mus musculus cDNA, 3 end/clone = UI-M-BH2.1-apz-g-10-0-UI/ | 1209.4 | P | 1703.4 | P | 1858.2 | P | 1317.3 |
| | | clone_end = 3/gb = AW125185/gi = 6100715/ug = Mm.24847/len = 215/STRA = for | | | | | | | |
| 7 | 96047_at | Cluster Incl U63146 Mus musculus retinol binding protein (RBP) mRNA, complete cds/cds = (0,605)/gb = U63146/ | 194.6 | P | 179.3 | P | 618.6 | P | 123.5 |
| | | gi = 1515449/ug = Mm 2605/len = 606/STRA = for | | | | | | | |
| 7 | 96083_s_at | Cluster Incl AI226368 uf07b12.y1 Mus musculus cDNA, 5 end/clone = IMAGE-1510655/clone_end = 5/gb = AI226368/ | 724.3 | P | 803.4 | P | 1241.3 | P | 738 |
| | | gi = 3809421/ug = Mm 26617/len = 340/STRA = for | | | | | | | |
| 7 | 96084_at | Cluster Incl AB017020 Mus musculus mRNA for JKTBP, complete cds/cds = (123,1028)/gb = AB017020/gi = 4514630/ | 557.5 | P | 267.8 | P | 500.9 | P | 135.5 |
| | | ug = Mm 26617/len = 1307/STRA = for | | | | | | | |
| 7 | 96633_s_at | Cluster Incl AA529583 vi41d07 r1 Mus musculus cDNA, 5 end/clone = IMAGE-906349/clone_end = 5/gb = AA529583/ | 1388.5 | P | 1427.6 | P | 1716 | P | 1500 |
| | | gi = 2272289/ug = Mm 27218/len = 546/STRA = for | | | | | | | |
| 7 | 96664_at | Cluster Incl AB025966 Mus musculus mRNA for KIAA312p, partial cds/cds = (0,1123)/gb = AB025966/gi = 5931572/ | 1088.6 | P | 1043 | P | 1113.6 | P | 806.1 |
| | | ug = Mm 27372/len = 1638/STRA = for | | | | | | | |
| 7 | 97211_at | Cluster Incl AI747444: ul15f07.x1 Mus musculus cDNA, 3 end/clone = IMAGE-2076901/clone_end = 3/gb = AI747444/ | 357.5 | P | 514.2 | P | 641.3 | P | 264.5 |
| | | gi = 5125708/ug = Mm 27714/len = 657/STRA = for | | | | | | | |
| 7 | 97240_g_at | Cluster Incl AI845106.UI-M-BG0-ahs-a-04-0-UI s1 Mus musculus cDNA, 3 end/clone = UI-M-BG0-ahs-a-04-0-UI/ | 1025.1 | P | 1200.7 | P | 1253 | P | 1129.2 |
| | | clone_end = 3/gb = AI845106/gi = 5489012/ug = Mm 27839/len = 141/STRA = for | | | | | | | |
| 7 | 97819_at | Cluster Incl AI843119 UI-M-AK1-aes-f-05-0-UI s1 Mus musculus cDNA, 3 end/clone = UI-M-AK1-aes-f-05-0-UI/ | 345.5 | P | 655.5 | P | 1038.3 | P | 494.9 |
| | | clone_end = 3/gb = AI843119/gi = 5477332/ug = Mm 282/len = 199/STRA = for | | | | | | | |
| 7 | 97864_at | Cluster Incl AW258842 um74d01 y1 Mus musculus cDNA, 5 end/clone = IMAGE-2300929/clone_end = 5/gb = AW258842/ | 195.6 | P | 265.5 | P | 304.4 | P | 254.4 |
| | | gi = 6631823/ug = Mm 28327/len = 321/STRA = for | | | | | | | |
| 7 | 97865_g_at | Cluster Incl AW258842 um74d01 y1 Mus musculus cDNA, 5 end/clone = IMAGE-2300929/clone_end = 5/gb = AW258842/ | 695 | P | 573.6 | P | 818.6 | P | 710.3 |
| | | gi = 6631823/ug = Mm 28327/len = 321/STRA = for | | | | | | | |
| 7 | 98937_at | Cluster Incl AW049795 UI-M-BH1-anm-1-07-0-UI s1 Mus musculus cDNA, 3 end/clone = UI-M-BH1-anm-f-07-0-UI/ | 865.8 | P | 1110.4 | P | 1128.9 | P | 1085.2 |
| | | clone_end = 3/gb = AW049795/gi = 5910324/ug = Mm 28689/len = 562/STRA = for | | | | | | | |
| 7 | 94448_at | Cluster Incl AJ006289: Mus musculus mRNA for bcl-10 protein/cds = (78,779)/gb = AJ006289/gi = 4049471/ug = Mm.28782/ | 484.8 | P | 457.2 | P | 995.7 | P | 576 |
| | | len = 1756/STRA = for | | | | | | | |
| 7 | 94516_f_at | Cluster Incl M55181: Preproenkephalin 2/cds = UNKNOWN/gb = M55181/gi = 201032/ug = Mm.2899/len = 1408/STRA = for | 1491.6 | P | 2222.6 | P | 2997.5 | P | 1773.9 |
| 7 | 95709_at | Cluster Incl AW012491 uo05f02 y1 Mus musculus cDNA, 5 end/clone = IMAGE-2582523/clone_end = 5/gb = AW012491/ | 1124 | P | 1318.8 | P | 1714.7 | P | 865 |
| | | gi = 5861269/ug = Mm 29703/len = 528/STRA = for | | | | | | | |
| 7 | 96254_at | Cluster Incl AB028272 Mus musculus hsp40 mRNA for heat shock protein 40, complete cds/cds = (139,1161)/ | 377.8 | P | 364.1 | P | 538.1 | P | 289.1 |
| | | gb = AB028272/gi = 6531981/ug = Mm 2982/len = 2279/STRA = for | | | | | | | |
| 7 | 96271_at | Cluster Incl AA914105 vy92c01.r1 Mus musculus cDNA, 5 end/clone = IMAGE-1313664/clone_end = 5/gb = AA914105/ | 1481.2 | P | 1554.6 | P | 1584.5 | P | 1332.2 |
| | | gi = 3053497/ug = Mm 29853/len = 415/STRA = for | | | | | | | |
| 7 | 96288_at | Cluster Incl AI648831 uk29f08.x1 Mus musculus cDNA, 3 end/clone = IMAGE-1970439/clone_end = 3/gb = AI648831/ | 393.3 | P | 416.9 | P | 472.5 | P | 438.6 |
| | | gi = 4729665/ug = Mm 29889/len = 526/STRA = for | | | | | | | |
| 7 | 96325_at | Cluster Incl AW124874 UI-M-BH2.1-apu-b-08-0-UI s1 Mus musculus cDNA, 3 end/clone = UI-M-BH2 1-apu-b-08-0-UI/ | 331.7 | P | 282.9 | P | 373.3 | P | 266.6 |
| | | clone_end = 3/gb = AW124874/gi = 6100404/ug = Mm 29949/len = 429/STRA = for | | | | | | | |
| 7 | 96336_at | Cluster Incl AI844626 UI-M-AL1-ahr-a-04-0-UI.s1 Mus musculus cDNA, 3 end/clone = UI-M-AL1-ahr-a-04-0-UI/ | 347.7 | P | 445.4 | P | 874.3 | P | 352.8 |
| | | clone_end = 3/gb = AI844626/gi = 5488532/ug = Mm.29975/len = 568/STRA = for | | | | | | | |
| 7 | 96841_at | Cluster Incl AW046627 UI-M-BH1-ald-c-09-0-UI s1 Mus musculus cDNA, 3 end/clone = UI-M-BH1-ald-c-09-0-UI/ | 325.2 | P | 399.4 | P | 691.9 | P | 434 |
| | | clone_end = 3/gb = AW046627/gi = 5907158/ug = Mm 30018/len = 268/STRA = for | | | | | | | |
| 7 | 96920_at | Cluster Incl AW125478: UI-M-BH2.2-aqm-c-03-0-UI s1 Mus musculus cDNA, 3 end/clone = UI-M-BH2 2-aqm-c-03-0-UI/ | 1057.6 | P | 1246 | P | 1669.5 | P | 1092.1 |
| | | clone_end = 3/gb = AW125478/gi = 6101008/ug = Mm 30156/len = 468/STRA = for | | | | | | | |
| 7 | 97450_s_at | Cluster Incl AA986258 uc74b03.y1 Mus musculus cDNA, 5 end/clone = IMAGE-1431341/clone_end = 5/gb = AA986258/ | 367.4 | P | 274.8 | P | 388 | P | 308.4 |
| | | gi = 3167582/ug = Mm 30250/len = 552/STRA = for | | | | | | | |
| 7 | 97462_at | Cluster Incl AI847584 UI-M-AP1-agg-a-06-0-UI s1 Mus musculus cDNA, 3 end/clone = UI-M-AP1-agg-a-06-0-UI/ | 732 | P | 677.8 | P | 762.6 | P | 583.6 |
| | | clone_end = 3/gb = AI847584/gi = 5491490/ug = Mm 3035/len = 340/STRA = for | | | | | | | |
| 7 | 97492_at | Cluster Incl AW121746: UI-M-BH2.3-aoe-c-09-0-UI s1 Mus musculus cDNA, 3 end/clone = UI-M-BH2 3-aoe-c-09-0-UI/ | 261.9 | P | 352.4 | P | 378.4 | P | 259.7 |
| | | clone_end = 3/gb = AW121746/gi = 6097081/ug = Mm 31076/len = 293/STRA = for | | | | | | | |
| 7 | 97496_f_at | Cluster Incl AW048944.UI-M-BH1-amo-e-07-0-UI.s1 Mus musculus cDNA, 3 end/clone = UI-M-BHI-amo-e-07-0-UI/ | 887.4 | P | 1350.9 | P | 1514.3 | P | 1298.5 |
| | | clone_end = 3/gb = AW048944/gi = 5909473/ug = Mm 3124/len = 296/STRA = for | | | | | | | |
| 7 | 97538_at | Cluster Incl M19279 Beta-glucuronidase structural/cds = (12,1958)/gb = M19279/gi = 193524/ug = Mm 3317/len = 2454/ | 163.6 | P | 173.5 | P | 248.9 | P | 160.3 |
| | | STRA = for | | | | | | | |
| 7 | 98084_at | Cluster Incl AI849834: UI-M-AL1-aho-h-09-0-UI s1 Mus musculus cDNA, 3 end/clone = UI-M-AL1-aho-h-09-0-UI/ | 1366.2 | P | 1315.7 | P | 1604.8 | P | 1230.8 |
| | | clone_end = 3/gb = AI849834/gi = 5493780/ug = Mm 34577/len = 464/STRA = for | | | | | | | |
| 7 | 98110_at | Cluster Incl AI853375: UI-M-BH0-air-e-09-0-UI s1 Mus musculus cDNA, 3 end/clone = UI-M-BH0-air-e-09-0-UI/ | 315.9 | P | 302.8 | P | 479.4 | P | 299.4 |
| | | clone_end = 3/gb = AI853375/gi = 5497281/ug = Mm 34810/len = 388/STRA = for | | | | | | | |
| 7 | 98593_at | Cluster Incl AJ006215: Mus musculus mRNA for CMP-N-acetylneuraminic acid synthetase/cds = (0,1298)/gb = AJ006215/ | 271.8 | P | 278.9 | P | 365.2 | P | 265.7 |
| | | gi = 3413319/ug = Mm.3820/len = 1687/STRA = for | | | | | | | |
| 7 | 99082_at | Cluster Incl L07063 Mus musculus FKBP65 binding protein mRNA, complete cds/cds = (93,1838)/gb = L07063/gi = 894161/ | 395.8 | P | 487.8 | P | 552.6 | P | 383.8 |
| | | ug = Mm 3894/len = 2597/STRA = for | | | | | | | |
| 7 | 99127_at | Cluster Incl X61506 Mouse E46 mRNA for E46 protein/cds = (198,1478)/gb = X61506/gi = 50794/ug = Mm 4098/len = 1668/ | 1199.6 | P | 1375.3 | P | 1759.8 | P | 1057.8 |
| | | STRA = for | | | | | | | |
| 7 | 99133_at | Cluster Incl X14309 Antigen identified by monoclonal antibodies 4F2/cds = (92,1672)/gb = X14309/gi = 49749/ug = Mm 4114/ | 1188.4 | P | 1125.9 | P | 1250 8 | P | 1178.6 |
| | | len = 1828/STRA = for | | | | | | | |
| 7 | 99563_at | Cluster Incl AW124681 UI-M-BH2.1-apw-g-06-0-UI s1 Mus musculus cDNA, 3 end/clone = UI-M-BH2 1-apw-g-06-0-UI/ | 483.9 | P | 537.9 | P | 569.9 | P | 552.5 |
| | | clone_end = 3/gb = AW124681/gi = 6100211/ug = Mm 42193/len = 421/STRA = for | | | | | | | |
| 7 | 99602_at | Cluster Incl AF064088 Mus musculus transcription factor GIF mRNA, complete cds/cds = (109,1548)/gb = AF064088/ | 486.8 | P | 424.6 | P | 884.8 | P | 570.9 |
| | | gi = 3139151/ug = Mm 4292/len = 3036/STRA = for | | | | | | | |
| 7 | 100094_at | Cluster Incl U88539 Mus musculus chromatin structural protein homolog Supt5hp (Supt5h) mRNA, complete cds/ | 548.7 | P | 582.4 | P | 641.8 | P | 542.2 |
| | | cds = (61,3309)/gb = U88539/gi = 2754751/ug = Mm 460/len = 3538/STRA = for | | | | | | | |
| 7 | 100115_at | Cluster Incl AW124128 UI-M-BH2.1-apr-d-07-0-UI s1 Mus musculus cDNA, 3 end/clone = UI-M-BH2 1-apr-d-07-0-UI/ | 267 | P | 364.8 | P | 459.3 | P | 377.5 |
| | | clone_end = 3/gb = AW124128/gi = 6099658/ug = Mm 46624/len = 357/STRA = for | | | | | | | |
| 7 | 100126_at | Cluster Incl AA967263 vz70e04 r1 Mus musculus cDNA, 5 end/clone = IMAGE-1331838/clone_end = 5/gb = AA967263/ | 582.3 | P | 480.7 | P | 702.2 | P | 528.1 |
| | | gi = 3141156/ug = Mm 4748/len = 432/STRA = for | | | | | | | |
| 7 | 100554_at | Cluster Incl AF053367 Mus musculus carboxyl terminal LIM domain protein (Ldb3) mRNA, complete cds/cds = (60,1040)/ | 366.8 | P | 714.4 | P | 803.7 | P | 497.6 |
| | | gb = AF053367/gi = 2996195/ug = Mm 5567/len = 1256/STRA = for | | | | | | | |
| 7 | 100555_at | Cluster Incl AI846152.UI-M-AN1-afj-h-05-0-UI.s2 Mus musculus cDNA, 3 end/clone = UI-M-AN1-afj-h-05-0-UI/ | 755.4 | P | 785.1 | P | 1237.8 | P | 843.2 |
| | | clone_end = 3/gb = AI846152/gi = 5490058/ug = Mm 56/len = 408/STRA = for | | | | | | | |
| 7 | 100557_g_at | Cluster Incl AW121930-UI-M-BH2.3-aoj-d-12-0-UI s1 Mus musculus cDNA, 3 end/clone = UI-M-BH2 3-aoj-d-12-0-UI/ | 1442.5 | P | 1105 | P | 1806.3 | P | 1366.5 |
| | | clone_end = 3/gb = AW121930/gi = 6097374/ug = Mm 5612/len = 467/STRA = for | | | | | | | |
| 7 | 100579_s_at | Cluster Incl U91848 Mus musculus clathrin light chain 2 mRNA, complete cds/cds = (108,815)/gb = U91848/gi = 1916987/ | 996.5 | P | 1093.2 | P | 1165.1 | P | 968.3 |
| | | ug = Mm 6072/len = 1089/STRA = for | | | | | | | |
| 7 | 100613_at | Cluster Incl AF030635: Mus musculus FK-506 binding protein homolog (SAM11) mRNA, complete cds/cds = (28,1095)/ | 855.8 | P | 1084.2 | P | 1180.3 | P | 919 |
| | | gb = AF030635/gi = 2623223/ug = Mm 6565/len = 1514/STRA = for | | | | | | | |
| 7 | 100622_at | Cluster Incl AF093857: untitled/cds = (49,723)/gb = AF093857/gi = 4139185/ug = Mm 6587/len = 1436/STRA = for | 627.7 | P | 769.6 | P | 1322.3 | P | 944.6 |
| 7 | 101078_at | Cluster Incl Y16258 Basigin/cds = (0,773)/gb = Y18258/gi = 2808471/ug = Mm 726/len = 1244/STRA = for | 2062.5 | P | 2567.6 | P | 2765.1 | | 2216.7 |
| 7 | 101086_f_at | Cluster Incl X63866 Mus musculus mRNA for cellular nucleic acid binding protein/cds = (104,616)/gb = X63866/gi = 50470/ | 1995.1 | P | 2051.6 | P | 2027.5 | P | 1675.2 |
| | | ug = Mm 7335/len = 1492/STRA = for | | | | | | | |
| 7 | 101088_f_at | Cluster Incl U20326 Mus musculus cellular nucleic acid binding protein mRNA, complete cds/cds = (119,655)/gb = U20326/ | 1838.7 | P | 1945.5 | P | 2071.9 | P | 1724 |
| | | gi = 854674/ug = Mm 7335/len = 1519/STRA = for | | | | | | | |
| 7 | 101095_at | Cluster Inc L23769 Microfibril-associated glycoprotein/cds = (80,631)/gb = L23769/gi = 440831/ug = Mm 7386/len = 1020/ | 874.9 | P | 1735.6 | P | 1993 .5 | P | 714.2 |
| | | STRA = for | | | | | | | |
| 7 | 101107_at | Cluster Incl U81829 Calumenin/cds = (35,982)/gb = U81829/gi = 2323276/ug = Mm 7515/len = 3147/STRA = for | 1330.2 | P | 1548.1 | P | 1714.9 | P | 1269.4 |
| 7 | 101110_at | Cluster Incl AF064749 Mus musculus type VI collagen alpha 3 subunit mRNA, complele cds/cds = (61,8034)/gb = AF064749/ | 2696 | P | 4155.8 | P | 4749.8 | P | 2944.3 |
| | | gi = 3236369/ug = Mm 7562/len = 8775/STRA = for | | | | | | | |
| 7 | 101499_at | Cluster Incl U94479 Mus musculus Integrin binding protein kinase mRNA, complete cds/cds = (113,1471)/gb = U94479/ | 918.5 | P | 1071.7 | P | 1211.2 | P | 921.3 |
| | | gi = 2739449/ug = Mm 8131/len = 1750/STRA = for | | | | | | | |
| 7 | 101514_at | Cluster Incl AF041433 Mus musculus bet3 (Bet3) mRNA, complete cds/cds = (82,624)/gb = AF041433/gi = 2791805/ | 347.1 | P | 602.4 | P | 536.8 | P | 541 |
| | | ug = Mm 8392/len = 753/STRA = for | | | | | | | |
| 7 | 101991_at | Cluster Incl D16215 Mouse mRNA for flavin-containing monooxygenase, complete cds/cds = (206,1804)/gb = D16215/ | 196.1 | P | 201.7 | P | 312.6 | P | 229.6 |
| | | gi = 286077/ug = Mm 976/len = 2126/STRA = rev | | | | | | | |
| 7 | 102044_at | Cluster Incl AF100777 Mus musculus connective tissue growth factor related protein WISP-1 (Wisp1) mRNA, complete cds/ | 202.7 | P | 190 | P | 260.7 | P | 201.6 |
| | | cds = (78,1181)/gb = AF100777/gi = 4028576/ug = Mm 10222/len = 1766/STRA = for | | | | | | | |
| 7 | 102322_at | Cluster Incl AF061017 UDP-glucose dehydrogenase/cds = (110,1591)/gb = AF061017/gi = 3127128/ug = Mm 10709/ | 1193.5 | P | 1338.3 | P | 2158.8 | P | 1879.6 |
| | | len = 2328/STRA = for | | | | | | | |
| 7 | 102395_at | Cluster Incl Z38110 M musculus PMP22 mRNA for peripheral myelin protein/cds = (0,482)/gb = Z38110/gi = 558353/ | 1022.7 | P | 1194.8 | P | 1492 | P | 830.6 |
| | | ug = Mm 1237/len = 483/STRA = for | | | | | | | |
| 7 | 102752_at | Cluster Incl AF072697 Mus musculus SHYC (Shyc) mRNA, complete cds/cds = (33,3794)/gb = AF072697/gi = 3293550/ | 917.9 | P | 1079.3 | P | 1141.5 | P | 1033.5 |
| | | ug = Mm.12912/len = 4112/STRA = for | | | | | | | |
| 7 | 102772_at | Cluster Incl J02995 Abelson murine leukemia oncogene/cds = (3565,4056)/gb = J02995/gi = 191566/ug = Mm 1318/ | 195 | P | 200.8 | P | 282.1 | P | 187.1 |
| | | len = 5058/STRA = for | | | | | | | |
| 7 | 102811_at | Cluster Incl X96639 Exostoses (multiple) 1/cds = (594,2831)/gb = X96639/gi = 1235558/ug = Mm 1424/len = 2965/STRA = for | 622.9 | P | 608.1 | P | 724.1 | P | 651.3 |
| 7 | 102983_at | Cluster Incl U58992.MAD homolog 1 (Drosophila)/cds = (70,1467)/gb = U58992/gi = 1518644/ug = Mm 15185/len = 1639/ | 378.5 | P | 401 | P | 470.7 | P | 373.4 |
| | | STRA = for | | | | | | | |
| 7 | 102984_g_at | Cluster Incl U58992: MAD homolog 1 (Drosophila)/cds = (70,1467)/gb = U58992/gi = 1518644/ug = Mm 15185/len = 1639/ | 889.5 | P | 930.5 | P | 1017.4 | P | 833 |
| | | STRA = for | | | | | | | |
| 7 | 103054_at | Cluster Incl U37500: Mus musculus RNA polymerase II largest subunit (RPB1) mRNA, complete cds/cds = (69,5969)/ | 489.6 | P | 568.7 | P | 674.2 | P | 527.3 |
| | | gb = U37500/gi = 2145090/ug = Mm.16533/len = 8270/STRA = rev | | | | | | | |
| 7 | 103100_at | Cluster Incl AI788543 uk46c09 x1 Mus musculus cDNA, 3 end/clone = IMAGE-1972048/clone_end = 3/gb = AI788543/ | 465.6 | P | 576.3 | P | 699.3 | P | 449.3 |
| | | gi-5336259/ug = Mm 17569/len = 656/STRA = for | | | | | | | |
| 7 | 103353_f_at | Cluster Incl D50834 Cytochrome P450. subfamily IV B, polypeptide 1/cds = (18,1553)/gb = D50834/gi = 854665/ | 377.9 | P | 670.1 | P | 558.9 | P | 527.2 |
| | | ug = Mm 1840/len = 1871/STRA = for | | | | | | | |
| 7 | 103379_at | Cluster Incl AW046579 UI-M-BH1-alb-e-03-0-UI s1 Mus musculus cDNA, 3 end/clone = UI-M-BH1-alb-e-03-0-UI/ | 124.8 | P | 101.8 | P | 150.3 | P | 125.2 |
| | | clone_end = 3/gb = AW046579/gi = 5907108/ug = Mm 18616/len = 301/STRA = rev | | | | | | | |
| 7 | 103608_at | Cluster Incl AI842638.UI-M-AQ1-aed-f-04-0-UI s1 Mus musculus cDNA, 3 end/clone = UI-M-AQ1-aed-f-04-0-UI/ | 290.6 | P | 342.1 | P | 370.9 | P | 302.5 |
| | | clone_end = 3/gb = AI842636/gi = 5476849/ug = Mm 20175/len = 311/STRA = for | | | | | | | |
| 7 | 104100_at | Cluster Incl AI845915: UI-M-AK1-aex-d-02-0-UI.s1 Mus musculus cDNA, 3 end/clone = UI-M-AK1-aex-d-02-0-UI/ | 1529.9 | P | 3175.4 | P | 3217.3 | P | 2255.1 |
| | | clone_end = 3/gb = AI845915/gi = 5489821/ug = Mm.21864/len = 208/STRA = for | | | | | | | |
| 7 | 104343_f_at | Cluster Incl AI845798-UI-M-AQ1-aeb-h-07-0-UI s1 Mus musculus cDNA, 3 end/clone = UI-M-AQ1-aeb-h-07-0-UI/ | 276.9 | P | 414.8 | P | 339.1 | P | 396.1 |
| | | clone_end = 3/gb = AI845798/gi = 5489704/ug = Mm 22526/len = 184/STRA = for | | | | | | | |
| 7 | 104587_at | Cluster Incl U69176: Laminin, alpha 4/cds = (386,5833)/gb = U69176/gi = 1568624/ug = Mm 2399/len = 5993/STRA = for | 393 | P | 506 | P | 637.3 | P | 410.1 |
| 7 | 104712_at | Cluster Incl L00039 Myelocytomalosis oncogene/cds = (15,1334)/gb = L00039/gi = 192621/ug = Mm 2444/len = 1707/ | 101.3 | P | 77.7 | P | 316. 5 | P | 103 |
| | | STRA = for | | | | | | | |
| 7 | 104756_at | Cluster Incl AA739415: vv53c01 r1 Mus musculus cDNA, 5 end/clone = IMAGE-1226112/clone_end = 5/gb = AA739415/ | 410.2 | P | 440.9 | P | 579.6 | P | 427.3 |
| | | gi = 2775601/ug = Mm.24788/len = 451/STRA = for | | | | | | | |
| 7 | 94394_at | Cluster Incl M21019: Harvey rat sarcoma oncogene, subgroup R/cds = (11,667)/gb = M21019/gi = 200676/ug = Mm 257/ | 57.7 | P | 267.6 | P | 361.1 | P | 193.3 |
| | | len = 949/STRA = rev | | | | | | | |
| 7 | 94415_at | Cluster Incl AA710439 vt42e01 r1 Mus musculus cDNA, 5 end/clone = IMAGE-1165752/clone_end = 5/gb = AA710439/ | 213.1 | P | 180.8 | P | 614.7 | P | 283.9 |
| | | gi = 2720357/ug = Mm 26147/len = 510/STRA = for | | | | | | | |
| 7 | 94942_at | Cluster Incl AI854108: UI-M-BH0-ajw-c-12-0-UI s1 Mus musculus cDNA, 3 end/clone = UI-M-BH0-ajw-c-12-0-UI/ | 184.5 | P | 404.6 | P | 313.9 | P | 363.9 |
| | | clone_end = 3/gb = AI854108/gi = 5498014/ug = Mm 26944/len = 581/STRA = rev | | | | | | | |
| 7 | 95609_at | Cluster Incl AA869927: vq19d11.r1 Mus musculus cDNA, 5 end/clone = IMAGE-1094709/clone_end = 5/gb = AA869927/ | 416.7 | P | 490.1 | P | 452.8 | P | 392.8 |
| | | gi = 2965372/ug = Mm 27987/len = 497/STRA = for | | | | | | | |
| 7 | 96178_at | Cluster Incl AI850636 UI-M-BG1-aik-e-04-0-UI.s1 Mus musculus cDNA, 3 end/clone = UI-M-BG1-aik-e-04-0-UI/ | 376.9 | P | 354.3 | P | 464.8 | P | 518.1 |
| | | clone_end = 3/gb = AI850636/gi = 5494542/ug = Mm 28426/len = 391/STRA = for | | | | | | | |
| 7 | 96733_at | Cluster Incl AW124597 UI-M-BH2 1-apt-h-01-0-UI s1 Mus musculus cDNA, 3 end/clone = UI-M-BH2 1-apt-h-01-0-UI/ | 534.1 | P | 406.2 | P | 744.4 | P | 585.2 |
| | | clone_end = 3/gb = AW124597/gi = 6100132/ug = Mm 28893/len = 499/STRA = for | | | | | | | |
| 7 | 96767_at | Cluster Incl AF098633 Mus musculus GLUT4 vesicle protein mRNA, partial cds/cds = (0,791)/gb = AF098633/gi = 4200441/ | 206.7 | P | 341.6 | P | 306 | P | 288.4 |
| | | ug = Mm 29010/len = 1192/STRA = rev | | | | | | | |
| 7 | 97359_at | Cluster Incl AI851160 UI-M-BH0-akg-f-01-0-UI.s1 Mus musculus cDNA, 3 end/clone = UI-M-BH0-akg-f-01-0-UI/ | 208.7 | P | 443 | P | 455.3 | P | 377.4 |
| | | clone_end = 3/gb = AI851160/gi = 5495031/ug = Mm 29649/len = 447/STRA = for | | | | | | | |
| 7 | 98474_r_at | Cluster Incl U83903: Tumor necrosis factor induced protein 6/cds = (63,890)/gb = U83903/gi = 2062474/ug = Mm 3509/ | 100.6 | P | 111 | P | 758.8 | P | 246.6 |
| | | len = 1605/STRA = for | | | | | | | |
| 7 | 98475_at | Cluster Incl U69262: Mus musculus matrilin-2 precursor mRNA, complete cds/cds = (250,3120)/gb = U69262/gi = 2072791/ | 182.1 | P | 244.9 | P | 288.2 | P | 256 |
| | | ug = Mm 3511/len = 3571/STRA = for | | | | | | | |
| 7 | 98508_s_at | Cluster Incl D84376: Mus musculus mRNA for Phosphatidic acid phosphatase, complete cds/cds = (286,1137)/gb = D84376/ | 253.3 | P | 357.3 | P | 572.8 | P | 353.3 |
| | | gi = 1487872/ug = Mm 35836/len = 1212/STRA = for | | | | | | | |
| 7 | 99031_at | Cluster Incl AI854005 UI-M-BH0-arv-h-09-0-UI.s1 Mus musculus cDNA, 3 end/clone = UI-M-BH0-arv-h-09-0-UI/ | 272.3 | P | 385.3 | P | 371.8 | P | 304.5 |
| | | clone_end = 3/gb = AI854005/gi = 5497911/ug = Mm.38930/len = 538/STRA = for | | | | | | | |
| 7 | 99038_at | Cluster Incl L24554 Mus musculus adenylosuccinate synthetase mRNA, complete cds/cds = (82,1452)/gb = L24554/ | 388.5 | P | 429.2 | P | 496.4 | P | 420 |
| | | gi = 404058/ug = Mm 39061/len = 1559/STRA = for | | | | | | | |
| 7 | 99039_g_at | Cluster Incl L24554 Mus musculus adenylosuccinate synthetase mRNA, complete cds/cds = (82,1452)/gb = L24554/ | 405.9 | P | 407.9 | P | 533.9 | P | 369.3 |
| | | gi = 404056/ug = Mm 39061/len = 1559/STRA = for | | | | | | | |
| 7 | 99535_at | Cluster Incl AW047630 UI-M-BH1-akm-g-06-0-UI s1 Mus musculus cDNA, 3 end/clone = UI-M-BH1-akm-g-06-0-UI/ | 472.6 | P | 334.9 | P | 1079.6 | P | 499.6 |
| | | clone_end = 3/gb = AW047630/gi = 5908159/ug = Mm 42187/len = 385/STRA = for | | | | | | | |
| 7 | 100442_at | Cluster Incl AA656541 vs22a03 r1 Mus musculus cDNA, 5 end/clone = IMAGE-1138924/clone_end = 5/gb = AA656541/ | 239.2 | P | 254.3 | P | 243.9 | P | 285.8 |
| | | gi = 2592695/ug = Mm 4810/len = 305/STRA = for | | | | | | | |
| 7 | 100466_f_at | Cluster Incl AA655823: vs48b07 r1 Mus musculus cDNA, 5 end/clone = IMAGE-1149493/clone_end = 5/gb = AA655823/ | 1161.3 | P | 1672.5 | P | 1741.2 | P | 1084.2 |
| | | gi = 2591977/ug = Mm.4923/len = 356/STRA = for | | | | | | | |
| 7 | 100974_at | Cluster Incl AI844250 UI-M-AL1-ahk-g-10-0-UI s1 Mus musculus cDNA, 3 end/clone = UI-M-AL1-ahk-g-10-0-UI/ | 444.4 | P | 330 | P | 465.7 | P | 464 |
| | | clone_end = 3/gb = AI844250/gi = 5488156/ug = Mm.6667/len = 237/STRA = rev | | | | | | | |
| 7 | 101921_at | Cluster Incl D86563: RAB4A, member RAS oncogene family/cds = (129,770)/gb = D86583/gi = 2706877/ug = Mm 9221/ | 180.8 | P | 215. 3 | P | 276.9 | P | 157.4 |
| | | len = 1385/STRA = for | | | | | | | |
| 7 | 103310_at | Cluster Incl AA220427 mv70b05 r1 Mus musculus cDNA, 5 end/clone = IMAGE-660369/clone_end = 5/gb = AA220427/ | 130.6 | P | 171 | P | 202. 9 | P | 120.6 |
| | | gi = 1838880/ug = Mm 21184/len = 261/STRA = for | | | | | | | |
| 7 | 103549_at | Cluster Incl AW061260: UI-M-BH1-amj-f-05-0-UI s2 Mus musculus cDNA, 3 end/clone = UI-M-BH1-amj-f-05-0-UI/ | 459.2 | P | 726.1 | P | 722.7 | P | 535.8 |
| | | clone_end = 3/gb = AW061260/gi = 6009011/ug = Mm 23742/len = 185/STRA = for | | | | | | | |
| 7 | 103555_at | Cluster Incl AI843520 UI-M-AO1-aeh-c-10-0-UI s1 Mus musculus cDNA, 3 end/clone = UI-M-AO1-aeh-c-10-0-UI/ | 2476.4 | P | 2579.3 | P | 2974.8 | P | 2687.7 |
| | | clone_end = 3/gb = AI843520/gi = 5477733/ug = Mm 23842/len = 387/STRA = for | | | | | | | |
| 7 | 104235_at | Cluster Incl AW122882 UI-M-BH2.1-apa-c-06-0-UI s1 Mus musculus cDNA, 3 end/clone = UI-M-BH2 1-apa-c-06-0-UI/ | 1265.6 | P | 995.6 | P | 1643.2 | P | 1233.4 |
| | | clone_end = 3/gb = AW122882/gi = 6098412/ug = Mm 28117/len = 316/STRA = for | | | | | | | |
| 7 | 104410_at | Cluster Incl AW124785: UI-M-BH2.1-aqa-h-06-0-UI s1 Mus musculus cDNA, 3 end/clone = UI-M-BH2 1-aqa-h-06-0-UI/ | 752.4 | P | 1112.5 | P | 1586.8 | P | 877.4 |
| | | clone_end = 3/gb = AW124785/gi = 6100315/ug = Mm 2883/len = 417/STRA = for | | | | | | | |
| 7 | 104464_a_at | Cluster Incl AI642389: vs90e02 x1 Mus musculus cDNA, 3 end/clone = IMAGE-1153562/clone_end = 3/gb = AI642389/ | 435.5 | P | 653.6 | P | 607.6 | P | 385.4 |
| | | gi = 4720864/ug = Mm 29644/len = 309/STRA = for | | | | | | | |
| 7 | 104494_at | Cluster Incl AI642098: ub75g10.x1 Mus musculus cDNA, 3 end/clone = IMAGE-1383618/clone_end = 3/gb = AI642098/ | 518.9 | P | 484.6 | P | 660.5 | P | 428.9 |
| | | gi = 4720573/ug = Mm 3038/len = 448/STRA = for | | | | | | | |
| 7 | 92925_at | Cluster Incl M61007 CCAAT/enhancer binding protein (C/EBP), beta/cds = (16,906)/gb = M61007/gi = 191775/ug = Mm 4883/ | 1682.8 | P | 1576 | P | 2715.2 | P | 2137.2 |
| | | len = 1416/STRA = rev | | | | | | | |
| 7 | 93440_at | Cluster Incl AA692530 vt59h11.r1 Mus musculus cDNA, 5 end/clone = IMAGE-1167429/clone_end = 5/gb = AA692530/ | 276.4 | P | 306.6 | P | 332.8 | P | 300.5 |
| | | gi = 2693468/ug = Mm 6636/len = 414/STRA = for | | | | | | | |
| 7 | 93482_at | Cluster Incl AI117835 uc41b01 r1 Mus musculus cDNA, 5 end/clone = IMAGE-1400521/clone_end = 5/gb = AI117835/ | 575.8 | P | 640.4 | P | 792.4 | P | 636.2 |
| | | gi = 3518159/ug = Mm 7128/len = 397/STRA = rev | | | | | | | |
| 7 | 93924_f_at | Cluster Incl M13443 Mouse alpha-tubulin isotype M-alpha-7 mRNA, complete cds/cds = (39,1391)/gb = M13443/gi = 202218/ | 945.8 | P | 1397.8 | P | 1297.8 | P | 1248 |
| | | ug = Mm 89866/len = 1424/STRA = for | | | | | | | |
| 7 | 97740_at | Cluster Incl AI642662 vw01d07.x1 Mus musculus cDNA, 3 end/clone = IMAGE-1230637/clone_end = 3/gb = AI642662/ | 201.2 | P | 147.1 | P | 317 | P | 143.5 |
| | | gi = 4721137/ug = Mm 3994/len = 307/STRA = for | | | | | | | |
| 7 | 100708_at | Cluster Incl X13605: Murine mRNA for replacement variant histone H3.3/cds = (73,483)/gb = X13605/gi = 51197/ | 3948.7 | P | 4894.9 | P | 5016.9 | P | 4269.1 |
| | | ug = Mm.18516/len = 1619/STRA = for | | | | | | | |
| 7 | 93842_at | Cluster Incl AI196645: ui53d07 y1 Mus musculus cDNA, 5 end/clone = IMAGE-1886125/clone_end = 5/gb = AI196645/ | 145.5 | P | 121.4 | P | 539.8 | P | 224.5 |
| | | gi = 3749251/ug = Mm 21622/len = 1009/STRA = for | | | | | | | |
| 7 | 93974_at | Cluster Incl AW212475 uo89c05.x1 Mus musculus cDNA, 3 end/clone = IMAGE-2649704/clone_end = 3/gb = AW212475/ | 248.4 | P | 212.1 | P | 974.3 | P | 480.1 |
| | | gi = 6518562/ug = Mm 21679/len = 230/STRA = for | | | | | | | |
| 7 | 93996_at | Cluster Incl X01026: Mouse mRNA fragment for repetitive element B2/cds = (0,119)/gb = X01026/gi = 53957/ug = Mm 21758/ | 198.4 | P | 249.3 | P | 493.7 | P | 285.5 |
| | | len = 354/STRA = for | | | | | | | |
| 7 | 95469_at | Cluster Incl AA734444 vr90g04.r1 Mus musculus cDNA, 5 end/clone = IMAGE-1136022/clone_end = 5/gb = AA734444/ | 141.6 | P | 203.5 | P | 231.1 | P | 209.6 |
| | | gi = 2756111/ug = Mm 24786/len = 252/STRA = for | | | | | | | |
| 7 | 96637_at | Cluster Incl U33005 Mus musculus tbc1 mRNA, complete cds/cds = (402,3827)/gb = U33005/gi = 988220/ug = Mm 27271/ | 220.9 | P | 373.4 | P | 404.5 | P | 298 |
| | | len = 4039/STRA = for | | | | | | | |
| 7 | 96703_at | Cluster Incl AB029448: Mus musculus mRNA for Dixin-1, complete cds/cds = (212,2539)/gb = AB029448/gi = 6520236/ | 635.4 | P | 984 | P | 998.1 | P | 628 |
| | | ug = Mm.27578/len = 2764/STRA = for | | | | | | | |
| 7 | 96713_at | Cluster Incl AF052453 3-phosphoadenosine 5-phosphosulfate synthase 2/cds = (130,1995)/gb = AF052453/gi = 3342265/ | 163 | P | 124.8 | P | 225.2 | P | 164.8 |
| | | ug = Mm 27646/len = 1996/STRA = for | | | | | | | |
| 7 | 98942_r_at | Cluster Incl AW125284: UI-M-BH2 1-apy-a-11-0-UI s1 Mus musculus cDNA, 3 end/clone = UI-M-BH2 1-apy-a-11-0-UI/ | 119.2 | P | 163.2 | P | 225.8 | P | 109.2 |
| | | clone_end = 3/gb = AW125284/gi = 6100814/ug = Mm.28697/len = 336/STRA = for | | | | | | | |
| 7 | 96283_at | Cluster Incl AI849180 UI-M-AJ1-agy-f-10-0-UI s1 Mus musculus cDNA, 3 end/clone = UI-M-AJ1-agy-f-10-0-UI/ | 567.8 | P | 762.7 | P | 904.3 | P | 661.1 |
| | | clone_end = 3/gb = AI849180/gi = 5493086/ug = Mm.29870/len = 397/STRA = for | | | | | | | |
| 7 | 96930_at | Cluster Incl AI844128 UI-M-AL1-ahj-d-06-0-UI.s1 Mus musculus cDNA, 3 end/clone = UI-M-AL1-ahj-d-06-0-UI/ | 238.8 | P | 247.2 | P | 340.3 | P | 241.5 |
| | | clone_end = 3/gb = AI844128/gi = 5488034/ug = Mm.30169/len = 328/STRA = for | | | | | | | |
| 7 | 97444_at | Cluster Incl AI844520 UI-M-AJ1-ahf-a-08-0-UI s2 Mus musculus cDNA, 3 end/clone = UI-M-AJ1-ahf-a-08-0-UI/ | 296.5 | P | 246.4 | P | 423.9 | P | 173.4 |
| | | clone_end = 3/gb = AI844520/gi = 5488426/ug = Mm.30241/len = 491/STRA = for | | | | | | | |
| 7 | 98626_at | Cluster Incl AW047503 UI-M-BH1-ama-b-05-0-UI s1 Mus musculus cDNA, 3 end/clone = UI-M-BH1-ama-b-05-0-UI/ | 318.3 | P | 379.5 | P | 552 | P | 325.1 |
| | | clone_end = 3/gb = AW047503/gi = 5908032/ug = Mm.38746/len = 264/STRA = for | | | | | | | |
| 7 | 100592_at | Cluster Incl AI929971 uI60a06 y1 Mus musculus cDNA, 5 end/clone = IMAGE-2123410/clone_end = 5/gb = AI929971/ | 720.1 | P | 956.9 | P | 1097.3 | P | 1006.9 |
| | | gi = 5666012/ug = Mm 63/len = 510/STRA = rev | | | | | | | |
| 7 | 102378_at | Cluster Incl U02487: Kras oncogene-associated gene/cds = (27,677)/gb = U02487/gi = 437199/ug = Mm.1209/len = 4420/ | 86.8 | P | 98.7 | P | 297.9 | P | 79.6 |
| | | STRA = for | | | | | | | |
| 7 | 102387_at | Cluster Incl AA799042 vn40d08 r1 Mus musculus cDNA, 5 end/clone = IMAGE-1023663/clone_end = 5/gb = AA799042/ | 624.3 | P | 592.8 | P | 805.6 | P | 583.9 |
| | | gi = 2861997/ug = Mm.12183/len = 350/STRA = for | | | | | | | |
| 7 | 103032_at | Cluster Incl AF038008: Mus musculus tyrosylprotein sulfotransferase-1 mRNA, complete cds/cds = (210,1322)/ | 171.9 | P | 261.6 | P | 280.4 | P | 169.5 |
| | | gb = AF038008/gi = 3046915/ug = Mm.16084/len = 1867/STRA = for | | | | | | | |
| 7 | 104389_at | Cluster Incl AW049360 UI-M-BH1-ane-a-10-0-UI s1 Mus musculus cDNA, 3 end/clone = UI-M-BH1-ane-a-10-0-UI/ | 214.3 | P | 277.8 | P | 393.5 | P | 278.8 |
| | | clone_end = 3/gb = AW049360/gi = 5909889/ug = Mm.22712/len = 358/STRA = for | | | | | | | |
| 7 | 94384_at | Cluster Incl X67644 M musculus gly96 mRNA/cds = UNKNOWN/gb = X67644/gi = 287803/ug = Mm 25613/len = 1938/ | 538.5 | P | 99.3 | A | 1274.2 | P | 935.5 |
| | | STRA = for | | | | | | | |
| 7 | 94948_at | Cluster Incl AF097511: Mus musculus zyxin related protein-1 (Zrp1) mRNA, complete cds/cds = (66,1508)/gb = AF097511/ | 249.2 | M | 349.9 | P | 425.7 | P | 415.4 |
| | | gi = 3851623/ug = Mm.27063/len = 1664/STRA = for | | | | | | | |
| 7 | 95037_at | Cluster Incl AW046639 UI-M-BH1-ald-d-10-0-UI s1 Mus musculus cDNA, 3 end/clone = UI-M-BH1-ald-d-10-0-UI/ | 197 | P | 260.8 | P | 444.5 | P | 285.5 |
| | | clone_end = 3/gb = AW046639/gi = 5907168/ug = Mm.27557/len = 338/STRA = for | | | | | | | |
| 7 | 96752_at | Cluster Incl M90551 Intercellular adhesion molecule/cds = (75,1688)/gb = M90551/gi = 459870/ug = Mm 28973/len = 2540/ | 106.9 | P | 119.5 | P | 539.4 | P | 169.3 |
| | | STRA = for | | | | | | | |
| 7 | 97404_at | Cluster Incl AW048244 UI-M-BH1-aly-b-04-0-UI.s1 Mus musculus cDNA, 3 end/clone = UI-M-BH1-aly-b-04-0-UI/ | 239.2 | A | 227.7 | P | 251.4 | P | 229.6 |
| | | clone_end = 3/gb = AW048244/gi = 5908773/ug = Mm 29901/len = 328/STRA = for | | | | | | | |
| 7 | 98988_at | Cluster Incl AA614971 Vo32e09 r1 Mus musculus cDNA, 5 end/clone = IMAGE-1051624/clone_end = 5/gb = AA614971/ | 138.5 | P | 146.8 | P | 1197.7 | P | 156.6 |
| | | gi = 2502199/ug = Mm 3732/len = 137/STRA = for | | | | | | | |
| 7 | 100977_at | Cluster Incl AA691492 vu15h09 r1 Mus musculus cDNA, 5 end/clone = IMAGE-1180769/clone_end = 5/gb = AA691492/ | 184.7 | M | 268.3 | P | 293 | P | 211.2 |
| | | gi = 2692430/ug = Mm 6691/len = 449/STRA = for | | | | | | | |
| 7 | 101834_at | Cluster Incl Z14249 Protein kinase, mitogen activated kinase 3/cds = UNKNOWN/gb = Z14249/gi = 50858/ug = Mm 8385/ | 591.9 | P | 613.3 | P | 623.6 | P | 443.7 |
| | | len = 1718/STRA = for | | | | | | | |
| 7 | 103311_at | Cluster Incl AW121299 UI-M-BH2.3-aog-f-02-0-UI.s1 Mus musculus cDNA, 3 end/clone = UI-M-BH2.3-aog-f-02-0-UI/ | 524.6 | P | 292 | P | 623 | P | 457.4 |
| | | clone_end = 3/gb = AW121299/gi = 6096632/ug = Mm 21199/len = 236/STRA = for | | | | | | | |
| 7 | 104019_at | Cluster Incl AI851068 UI-M-BH0-ajv-h-07-UI s1 Mus musculus cDNA, 3 end/clone = UI-M-BH0-ajv-h-07-0-UI/ | 83.9 | P | 74.4 | P | 162.4 | P | 78.4 |
| | | clone end = 3/gb = AI851068/gi = 5494974/ug = Mm 26936/len = 441/STRA = for | | | | | | | |
| 7 | 100277_at | Cluster Incl X69619 lnhibin beta-A/cds = (172,1446)/gb = X69619/gi = 50145/ug = Mm 8042/len = 1529/STRA = for | 277.6 | P | 244.3 | P | 533.9 | P | 303.1 |
| 7 | 93062_at | Cluster Incl AW045881 UI-M-BH1-aky-h-01-0-UI.s1 Mus musculus cDNA, 3 end/clone = UI-M-BH1-aky-h-01-0-UI/ | 442.4 | P | 669 | P | 570.5 | P | 401.3 |
| | | clone end = 3/gb = AW045881/gi = 5906410/ug = Mm.1555/len = 561/STRA = for | | | | | | | |
| 7 | 93550_at | Cluster Incl D88792: Mouse mRNA for double LIM protein-1, complete cds/cds = (6,587)/gb = D88792/gi = 1695701/ | 363.8 | P | 429.5 | P | 670.9 | P | 209.5 |
| | | ug = Mm 2020/len = 822/STRA = for | | | | | | | |
| 7 | 96035_at | Cluster Incl L47335 Mus musculus branched chain alpha ketoacid decarboxylase E1a subunit mRNA, complete cds/ | 108.4 | M | 204.2 | P | 286.2 | P | 219.7 |
| | | cds = (17,1345)/gb = L47335/gi = 992564/ug = Mm 25848/len = 1681/STRA = for | | | | | | | |
| 7 | 95669_g_at | Cluster Incl AI840972 UI-M-AK0-adk-e-04-0-UI.s1 Mus musculus cDNA, 3 end/clone = UI-M-AK0-adk-e-04-0-UI/ | 326.6 | P | 415.2 | P | 454.3 | P | 332.2 |
| | | clone_end = 3/gb = AI840972/gi = 5475185/ug = Mm.29580/len = 456/STRA = for | | | | | | | |
| 7 | 96885_at | Cluster Incl AW122271.UI-M-BH2 2-aov-h-12-0-UI s1 Mus musculus cDNA, 3 end/clone = UI-M-BH2 2-aov-h-12-0-UI/ | 467.1 | P | 590.2 | P | 745.9 | P | 272.1 |
| | | clone_end = 3/gb = AW122271/gi = 6097746/ug = Mm 30083/len = 240/STRA = for | | | | | | | |
| 7 | 99167_at | Cluster Incl AW122550 UI-M-BH2.2-aox-d-08-0-UI.s1 Mus musculus cDNA, 3 end/clone = UI-M-BH2 2-aox-d-08-0-UI/ | 224.3 | P | 246.1 | P | 271.7 | P | 187.6 |
| | | clone_end = 3/gb = AW122550/gi_6098074/ug = Mm 41687/len = 462/STRA = for | | | | | | | |
| 7 | 103037_at | Cluster Incl U8366 Mus musculus cardiotrophin-1 mRNA complete cds/cds = (19,630)/gb = 18366)/gi = 710331 | 79.1 | P | 56.5 | M | 396.2 | P | 82.5 |
| | | ug = Mm 1616/len = 1345/stra = STRA = for | | | | | | | |
| 7 | 103044_g_at | Cluster Incl Z35294: M. musculus MTCP-1 gene/cds = (540,746)/gb = Z35294/gi = 603859/ug = Mm.16366/len = 954/ | 371 | P | 512.1 | P | 569.9 | P | 541.1 |
| | | STRA = for | | | | | | | |
| 7 | 103579_at | Cluster Incl X53247 M musculus EN-7 mRNA/cds = (36,614)/gb = X53247/gi = 50836/ug = Mm 1972/len = 1255/STRA = for | 875.1 | P | 780.4 | P | 903.6 | P | 917.5 |
| 7 | 104306_at | Cluster Incl AI847886 UI-M-AM1-afu-g-07-0-UI.s1 Mus musculus cDNA, 3 end/clone = UI-M-AM1-afu-g-07-0-UI/ | 284.8 | P | 339.6 | P | 351.6 | P | 294.6 |
| | | clone_end = 3/gb = AI847886/gi = 5491792/ug = Mm 22369/len = 392/STRA = for | | | | | | | |
| 7 | 94332_at | Cluster Incl AI882555 ub97f03 r1 Mus musculus cDNA, 5 end/clone = IMAGE-1396445/clone_end = 5/gb = AI882555/ | 73.1 | A | 102 | P | 121.7 | P | 109.1 |
| | | gi = 5567644/ug = Mm 2490/len = 329/STRA = for | | | | | | | |
| 7 | 94377_at | Cluster Incl AI854793: UI-M-BH0-aka-f-02-0-UI s1 Mus musculus cDNA, 3 end/clone = UI-M-BH0-aka-f-02-0-UI/ | 39.1 | P | 72.2 | P | 189.7 | P | 41.7 |
| | | clone_end = 3/gb = AI854793/gi = 5498699/ug = Mm 25547/len = 359/STRA = for | | | | | | | |
| 7 | 97353_at | Cluster Incl AI837497: UI-M-AL0-abr-g-08-0-UI s1 Mus musculus cDNA, 3 end/clone = UI-M-AL0-abr-g-08-0-UI/ | 54.6 | A | 131 | P | 202.4 | P | 97 |
| | | clone_end = 3/gb = AI837497/gi = 5471710/ug = Mm 29629/len = 224/STRA = for | | | | | | | |
| 7 | 98492_at | Cluster Incl AA920419 vz26a09 r1 Mus musculus cDNA, 5 end/clone = IMAGE-1327576/clone_end = 5/gb = AA920419/ | 712.3 | P | 824.9 | P | 992.7 | P | 808.2 |
| | | gi = 3067198/ug = Mm 35600/len = 371/STRA = for | | | | | | | |
| 7 | 99015_at | Cluster Incl U33626 Promyelocytic leukemia/cds = (86,2512)/gb = U33626/gi = 995828/ug = Mm 38497/len = 3760/STRA = for | 313.2 | P | 349.2 | A | 449.2 | P | 198.4 |
| 7 | 99444_at | Cluster Incl AJ250490.Mus musculus mRNA for receptor activity modifying protein 2 (Ramp2 gene)/cds = (30,599)/ | 143.7 | P | 250.2 | P | 320.6 | P | 239.8 |
| | | gb = AJ250490/gi = 6119626/ug = Mm 40358/len = 808/STRA = for | | | | | | | |
| 7 | 102736_at | Cluster Incl M19681 Small inducible cytokine A2/cds = (82,528)/gb = M19681/gi = 193486/ug = Mm.145/len = 798/STRA = for | 56 | P | −0.9 | P | 1319.2 | P | 426.3 |
| 7 | 103690_at | Cluster Incl AW125574 UI-M-BH2.2-aqj-c-05-0-UI.s1 Mus musculus cDNA, 3 end/clone = UI-M-BH2.2-aqj-c-05-0-UI/ | 140.7 | P | 116.7 | P | 253.1 | P | 142.4 |
| | | clone_end = 3/gb = AW125574/gi = 6101104/ug = Mm 23955/len = 566/STRA = rev | | | | | | | |
| 7 | 93376_at | Cluster Incl AA673486 vp49f04 r1 Mus musculus cDNA, 3 end/clone = IMAGE-1080031/clone_end = 3/gb = AA673486/ | 106.5 | P | 139.9 | P | 178 | P | 160.2 |
| | | gi = 2646768/ug = Mm 6156/len = 477/STRA = rev | | | | | | | |
| 7 | 102114_f_at | Cluster Incl AI326963 mj76a06.x1 Mus musculus cDNA, 3 end/clone = IMAGE-482002/clone_end = 3/gb = AI326963/ | 331.4 | P | 260.1 | P | 598 | P | 321.4 |
| | | gi = 4061392/ug = Mm 42609/len = 479/STRA = rev | | | | | | | |
| 7 | 93301_at | Cluster Incl AW124624 UI-M-BH2 1-apw-b-05-0-UI s1 Mus musculus cDNA, 3 end/clone = UI-M-BH2 1-apw-b-05-0-UI/ | 127 | P | 87 | A | 200.6 | P | 153.4 |
| | | clone_end = 3/gb = AW124624/gi = 6100154/ug = Mm 18230/len = 416/STRA = for | | | | | | | |
| 7 | 93563_s_at | Cluster Incl AB017202: Mus musculus mRNA for entactin-2, complete cds/cds = (43,4254)/gb = AB017202/gi = 3513367/ | 114.3 | P | 107.5 | P | 195.1 | P | 84.1 |
| | | ug = Mm 20348/len = 5094/STRA = for | | | | | | | |
| 7 | 96618_at | Cluster Incl AW045637.UI-M-BH1-alg-f-02-0-UI s1 Mus musculus cDNA, 3 end/clone = UI-M-BH1-alg-f-02-0-UI/ | 102 | A | 145.6 | P | 156.1 | P | 114.4 |
| | | clone_end = 3/gb = AW045637/gi = 5906166/ug = Mm 27131/len = 371/STRA = for | | | | | | | |
| 7 | 96640_at | Cluster Incl AI644158 vw55c12 x1 Mus musculus cDNA, 3 end/clone = IMAGE-1247734/clone_end = 3/gb = AI644158/ | 243.6 | A | 254.9 | A | 478.6 | P | 322.3 |
| | | gi = 4722633/ug = Mm 27274/len = 458/STRA = for | | | | | | | |
| 7 | 96843_at | Cluster Incl AF068116 Mus musculus eIF4E-like protein 4E-LP mRNA, complete cds/cds = (0,737)/gb = AF068116/ | 288.5 | P | 267.1 | P | 422 | P | 340.6 |
| | | gi = 3201957/ug = Mm 3002/len = 804/STRA = for | | | | | | | |
| 7 | 99603_g_at | Cluster Incl AF064088 Mus musculus transcription factor GIF mRNA, complete cds/cds = (109,1548)/gb = AF064088/ | 194.1 | M | 205.9 | P | 539.9 | P | 273.8 |
| | | gi = 3139151/ug = Mm 4292/len = 3036/STRA = for | | | | | | | |
| 7 | 97360_at | Cluster Incl AI841689 UI-M-AO0-acg-h-11-0-UI s1 Mus musculus cDNA, 3 end/clone = UI-M-AO0-acg-h-11-0-UI/ | 95.8 | P | 71.3 | P | 141.2 | P | 103 |
| | | clone_end = 3/gb = AI841689/gi = 5475902/ug = Mm 29658/len = 337/STRA = for | | | | | | | |
| 7 | 99930_s_at | Cluster Incl U57325 Mus musculus PS-2short mRNA, complete cds/cds = (124,579)/gb = U57325/gi = 2315275/ | 41.4 | A | 65 | P | 129.6 | P | 90.9 |
| | | ug = Mm 4338/len = 1146/STRA = for | | | | | | | |
| 7 | 103308_at | Cluster Incl AI450597: mq87e05 x1 Mus musculus cDNA, 3 end/clone = IMAGE-585728/clone_end = 3/gb = AI450597/ | 328.9 | P | 1450.8 | P | 1411 | A | 614 |
| | | gi = 4298115/ug = Mm 21144/len = 350/STRA = for | | | | | | | |
| 7 | 93068_r_at | Cluster Incl U62674 Mus musculus histone H2a 2-615 (H2a-615), and histone H3.2-615 (H3-615) genes, complete cds/ | 282.5 | P | 281.5 | P | 709 | P | 458.8 |
| | | cds = (43,435)/gb = U62674/gi = 1458142/ug = Mm 15686/len = 515/STRA = for | | | | | | | |
| 7 | 93328_at | Cluster Incl X57437: Histidine decarboxylase cluster/cds = (74,2062)/gb = X57437/gi = 52903/ug = Mm 18603/len = 2371/ | −22 | A | 146.1 | M | 1852 | P | 535.4 |
| | | STRA = for | | | | | | | |
| 7 | 94840_at | Cluster Incl U05837 Hexosaminidase A/cds = (150,1736)/gb = U05837/gi = 2182207/ug = Mm 2284/len = 1960/STRA = for | 179.1 | P | 192.2 | M | 380.3 | P | 125.6 |
| 7 | 103433_at | Cluster Incl AI846077 UI-M-AN1-afj-a-02-0-UI s2 Mus musculus cDNA, 3 end/clone = UI-M-AN1-afj-a-02-0-UI/ | 33 | A | 127.4 | P | 352.4 | P | 141.8 |
| | | clone_end = 3/gb = AI846077/gi = 5490023/ug = Mm 19065/len = 296/STRA = for | | | | | | | |
| 7 | 103563_at | Cluster Incl AW125713 UI-M-BH2 2-aql-c-12-0-UI s1 Mus musculus cDNA, 3 end/clone = UI-M-BH2 2-aql-c-12-0-UI/ | 172.2 | P | 34.2 | P | 178.3 | P | 14.9 |
| | | clone_end = 3/gb = AW125713/gi = 6101283/ug = Mm 19126/len = 445/STRA = for | | | | | | | |
| 7 | 104516_at | Cluster Incl U82758 Mus musculus lung-specific membrane protein mRNA, complete cds/cds = (21,677)/gb = U82758/ | 1.8 | A | 144.3 | P | 434.5 | P | 142.4 |
| | | gi = 3342229/ug = Mm 22768/len = 1319/STRA = for | | | | | | | |
| 7 | 102926_at | Cluster Incl AB008833 Mus musculus mRNA for GFRalpha-3, complete cds/cds = (45,1238)/gb = AB008833/gi = 2627159/ | 328 | P | 440.5 | P | 382 | M | 286.7 |
| | | ug = Mm.16520/len = 1867/STRA = for | | | | | | | |
| 7 | 92731_at | Cluster Incl X83601: Pentaxin related gene/cds = (99,1244)/gb = X83601/gi = 1181243/ug = Mm 4663/len = 1841/STRA = for | 22.5 | A | 11 | P | 439.2 | P | 172.1 |
| 7 | 94761_at | Cluster Incl X70058: Small Inducible cytokine A7/cds = (70,342)/gb = X70058/gi = 437872/ug = Mm 16091/len = 774/ | 101.5 | P | 96.8 | M | 632.9 | P | 283.1 |
| | | STRA = for | | | | | | | |
| 7 | 102100_f_at | Cluster Incl AI845835: UI-M-AO1-aeg-d-01-0-UI s1 Mus musculus cDNA, 3 end/clone = UI-M-AO1-aeg-d-01-0-UI/ | 23.1 | P | 87.4 | P | 67.4 | P | 28.5 |
| | | clone_end = 3/gb = AI845835/gi = 5489741/ug = Mm 42439/len = 269/STRA = rev | | | | | | | |
| 7 | 100521_at | Cluster Incl D32137 Mouse mRNA for oviducl-specific glycoprotein (MOGP), complete cds/cds = (14,2179)/gb = D32137/ | 142.5 | M | 212.7 | P | 180 | A | 42.3 |
| | | gi = 806523/ug = Mm 5110/len = 2525/STRA = rev | | | | | | | |
| 7 | 96131_at | Cluster Incl AI840116: UI-M-AL0-abt-h-05-0-UI s1 Mus musculus cDNA, 3 end/clone = UI-M-AL0-abt-h-05-0-UI/ | 269.9 | A | 270.5 | P | 260 | P | 319.8 |
| | | clone_end = 3/gb = AI840116/gi = 5474329/ug = Mm.28140/len = 348/STRA = for | | | | | | | |
| 7 | 104508_at | Cluster Incl AA756506 vv53h12 r1 Mus musculus cDNA, 5 end/clone = IMAGE-1226183/clone_end = 5/gb = AA756506/ | 284.5 | P | 284 | A | 309.3 | P | 297.6 |
| | | gi = 2803704/ug = Mm 30805/len = 473/STRA = for | | | | | | | |
| 7 | 104647_at | Cluster Incl M88242: Mouse glucocortoid-regulated inflammatory prostaglandin G/H synthase (griPGHS) mRNA, complete | −12 | A | 12.8 | A | 166.6 | P | 76 |
| | | cds/cds = (100,1914)/gb = M88242/gi = 193637/ug = Mm 3137/len = 4152/STRA = for | | | | | | | |
| 7 | 93153_at | Cluster Incl AW124433: UI-M-BH2.1-apo-b-10-0-UI.s1 Mus musculus cDNA, 3 end/clone = UI-M-BH2.1-apo-b-10-0-UI/ | 148.9 | P | 195.7 | A | 361.5 | P | 161.2 |
| | | clone_end = 3/gb = AW124433/gi = 6100003/ug = Mm.5258/len = 526/STRA = rev | | | | | | | |
| 7 | 95956_at | Cluster Incl C76923: C76923 Mus musculus cDNA, 3 end/clone = J0023A02/clone_end = 3/gb = C76923/gi = 2979405/ | 100.6 | A | 82.1 | A | 153.5 | P | 87.4 |
| | | ug = Mm 24933/len = 429/STRA = rev | | | | | | | |
| 7 | 97103_at | Cluster Incl AF031380 Mus musculus KOI-4 gene, partial cds/cds = (0,102)/gb = AF031380/gi = 2623675/ug = Mm 32715/ | −74.4 | P | −76.5 | A | 38.7 | P | −41.7 |
| | | len = 103/STRA = rev | | | | | | | |
| 7 | 92549_at | Cluster Incl U97170 Mus musculus protein kinase inhibitor gamma (PKIg) mRNA, complete cds/cds = (217,447)/ | 308.5 | A | 326.5 | A | 609.6 | P | 349 |
| | | gb = U97170/gi = 3004635/ug = Mm 10091/len = 1064/STRA = for | | | | | | | |
| 7 | 93985_at | Cluster Incl AW120868: UI-M-BH2.3-aoa-g-07-0-UI s1 Mus musculus cDNA, 3 end/clone = UI-M-BH2.3-aoa-g-07-0-UI/ | −89.1 | A | −53.2 | A | 4.2 | P | −49.4 |
| | | clone_end = 3/gb = AW120868/gi = 6096201/ug = Mm 21712/len = 284/STRA = for | | | | | | | |
| 7 | 96605_at | Cluster Incl AI787183: u86a11 y1 Mus musculus cDNA, 5 end/clone = IMAGE-1889276/clone_end = 5/gb = AI787183/ | −9.7 | M | 33.4 | A | 168.8 | P | 112.9 |
| | | gi = 5334899/ug = Mm 27061/len = 245/STRA = for | | | | | | | |
| 7 | 97811_at | Cluster Incl AI844507: UI-M-AJ1-ahe-g-11-0-UI s1 Mus musculus cDNA, 3 end/clone = UI-M-AJ1-ahe-g-11-0-UI/ | 6 | P | 147.6 | A | 123.7 | P | 62.3 |
| | | clone_end = 3/gb = AI844507/gi = 5488413/ug = Mm 28163/len = 299/STRA = for | | | | | | | |
| 7 | 94517_r_at | Cluster Incl M55181 Preproenkephalin 2/cds = UNKNOWN/gb = M55181/gi = 201032/ug = Mm 2899/len = 1408/STRA = for | 195.9 | A | 440.7 | A | 552.2 | P | 396.9 |
| 7 | 102326_at | Cluster Incl AB002664 Mus musculus mRNA for p67phox, complete cds/cds = (96,1673)/gb = AB002664/gi = 3061283/ | 93.8 | M | 90.9 | P | 102.3 | P | 78.2 |
| | | ug = Mm.10729/len = 1813/STRA = for | | | | | | | |
| 7 | 96203_at | Cluster Incl AI847646.UI-M-AP1-agg-g-07-0-UI s1 Mus musculus cDNA, 3 end/clone = UI-M-AP1-agg-g-07-0-UI/ | 20.5 | P | 46.1 | P | 87.6 | P | 19.6 |
| | | clone_end = 3/gb = AI847646/gi = 5491552/ug = Mm 28623/len = 226/STRA = for | | | | | | | |
| 7 | 99486_at | Cluster Incl X55038 Centromere autoantigen B/cds = (0,1799)/gb = X55038/gi = 50381/ug = Mm 41454/len = 1800/STRA = for | 112.8 | A | 269.2 | P | 350.6 | P | 297.6 |
| 7 | 92534_at | Cluster Incl U10551 GTP binding protein (gene overexpressed in skeletal muscle)/cds = (108,995)/gb = U10551/gi = 511464/ | 58.3 | A | 114.6 | P | 188.3 | P | 51.2 |
| | | ug = Mm 4362/len = 1981/STRA = for | | | | | | | |
| 7 | 92688_at | Cluster Incl X57199 Acid phosphatase 2, lysosomal/cds = (0,1265)/gb = X57199/gi = 52870/ug = Mm 45570/len = 1266/ | 201.8 | P | 223.9 | P | 247.1 | P | 175.8 |
| | | STRA = for | | | | | | | |
| 7 | 93656_g_at | Cluster Incl X95316 M musculus USF1 (exons 2 to 10)/cds = (214,1146)/gb = X95316/gi = 1765921/ug = Mm 8/len = 1902/STRA = | 290.6 | A | 262.2 | P | 557.4 | P | 252 |
| | | for | | | | | | | |
| 7 | 94778_at | Cluster Incl U96401: Mus musculus aldehyde dehydrogenase Ahd-2-like mRNA, complete cds/cds = (44,1549)/gb = U96401/ | 155.9 | P | 200.9 | A | 333.4 | P | 161.8 |
| | | gi = 2289239/ug = Mm.16773/len = 2060/STRA = for | | | | | | | |
| 7 | 95380_at | Cluster Incl L19057 Non MHC restricted killing associated/cds = (126,1322)/gb = L19057/gi = 309076/ug = Mm 2299/ | 63.3 | P | 59.7 | P | 70.9 | P | 55.9 |
| | | len = 1930/STRA = for | | | | | | | |
| 7 | 99835_at | Cluster Incl AF017128 Fos-like antigen 1/cds = (253,1074)/gb = AF017128/gi = 2407190/ug = Mm 6215/len = 1705/STRA = for | −31.4 | A | 50.8 | A | 661.2 | P | 101.8 |
| 8 | 92574_at | Cluster Incl AI851046 UI-M-BH0-ajv-f-05-0-UI s1 Mus musculus cDNA, 3 end/clone = UI-M-BH0-ajv-f-05-0-UI/ | 1103.1 | P | 1322.3 | P | 1089.6 | P | 1132.2 |
| | | clone_end = 3/gb = AI851046/gi = 5494952/ug = Mm 10406/len = 475/STRA = for | | | | | | | |
| 8 | 92615_at | Cluster Incl AI853615 UI-M-BH0-ajp-f-09-0-UI s1 Mus musculus cDNA, 3 end/clone = UI-M-BH0-ajp-f-09-0-UI/ | 1106.2 | P | 1433.3 | P | 1059.2 | P | 1081.4 |
| | | clone_end = 3/gb = AI853615/gi = 5497521/ug = Mm 1103/len = 542/STRA = for | | | | | | | |
| 8 | 92625_at | Cluster Incl X68193: M. musculus mRNA for nucleoside diphoshate kinase B/cds = (19,477)/gb = X68193/gi = 53353/ | 3712.2 | P | 4154 | P | 3157.7 | P | 3876.2 |
| | | ug = Mm.1125/len = 612/STRA = for | | | | | | | |
| 8 | 93026_at | Cluster Incl AW124337.UI-M-BH2 1-apq-f-08-0-UI.s1 Mus musculus cDNA, 3 end/clone = UI-M-BH2.1-apq-f-08-0-UI/ | 1575.3 | P | 1321.2 | P | 1187.8 | P | 1628.5 |
| | | clone_end = 3/gb = AW124337/gi = 6099867/ug = Mm 14796/len = 243/STRA = rev | | | | | | | |
| 8 | 93071_at | Cluster Incl X99644: M musculus mRNA for TIF1 beta protein/cds = (168,2672)/gb = X99644/gi = 1480113/ug = Mm 15701/ | 1997 | P | 2404.6 | P | 1896.2 | P | 1820.3 |
| | | len = 2858/STRA = for | | | | | | | |
| 8 | 93270_at | Cluster Incl AI839918 UI-M-AH0-acw-f-01-0-UI s1 Mus musculus cDNA, 3 end/clone = UI-M-AH0-acw-f-01-0-UI/ | 918.7 | P | 1075.8 | P | 977.4 | P | 974.9 |
| | | clone_end = 3/gb = AI839918/gi = 5474131/ug = Mm 17403/len = 429/STRA = for | | | | | | | |
| 8 | 93295_at | Cluster Incl Z31555 M musculus (129/Sv) Ccte mRNA for CCT (chaperonin containing TCP-1) epsilon subunit/ | 2474.1 | P | 2076.5 | P | 2146.7 | P | 2131.8 |
| | | cds = (99,1724)/gb = Z31555/gi = 468549/ug = Mm 1813/len = 1769/STRA = for | | | | | | | |
| 8 | 93305_f_at | Cluster Incl AF053724: Mus musculus endobrevin mRNA, complete cds/cds = (154,459)/gb = AF053724/gi = 2982367/ | 979.7 | P | 960 | P | 695.2 | P | 698.8 |
| | | ug = Mm 1838/len = 597/STRA = for | | | | | | | |
| 8 | 93340_f_at | Cluster Incl AF043120: Mus musculus beta prime coatomer protein mRNA, partial cds/cds = (0,1352)/gb = AF043120/ | 323.6 | P | 400.7 | P | 356.7 | P | 379.1 |
| | | gi = 2809536/ug = Mm.18729/len = 1615/STRA = for | | | | | | | |
| 8 | 93358_at | Cluster Incl AI836451 UI-M-AI0-aak-e-05-0-UI.s1 Mus musculus cDNA, 3 end/clone = UI-M-AI0-aak-e-05-0-UI/ | 1124.4 | P | 1187.4 | P | 933.8 | P | 1068.7 |
| | | clone_end = 3/gb = AI836451/gi = 5470664/ug = Mm.18924/len = 368/STRA = for | | | | | | | |
| 8 | 93533_at | Cluster Incl AI853651 UI-M-BH0-ajq-b-03-0-UI s1 Mus musculus cDNA, 3 end/clone = UI-M-BH0-ajq-b-03-0-UI/ | 644.6 | P | 619.2 | P | 567.9 | P | 562.3 |
| | | clone_end = 3/gb = AI853651/gi = 5497557/ug = Mm 1985/len = 445/STRA = for | | | | | | | |
| 8 | 93557_at | Cluster Incl U43285 Selenophosphate synthetase 2/cds = (137,1495)/gb = U43285/gi = 1815623/ug = Mm.20294/len = 2202/ | 215.1 | P | 236.9 | P | 251.6 | P | 253.9 |
| | | STRA = for | | | | | | | |
| 8 | 93581_at | Cluster Incl AI845121 UI-M-BG0-ahs-b-12-0-UI.s1 Mus musculus cDNA, 3 end/clone = UI-M-BG0-ahs-b-12-0-UI/ | 2147 | P | 2502.6 | P | 1941.5 | P | 2054.7 |
| | | clone_end = 3/gb = AI845121/gi = 5489027/ug = Mm 2060/len = 361/STRA = for | | | | | | | |
| 8 | 93593_f_at | Cluster Incl U87948 Epithelial membrane protein 3/cds = (201,692)/gb = U87948/gi = 2358253/ug = Mm 20829/len = 695/ | 1407.1 | P | 1869 | P | 1535.2 | P | 1555.1 |
| | | STRA = for | | | | | | | |
| 8 | 93596_i_at | Cluster Incl AA624586 vn82e11.r1 Mus musculus cDNA, 5 end/clone = IMAGE-1038476/clone_end = 5/gb = AA624588/ | 1490.2 | P | 1736.8 | P | 1298.6 | P | 1417.8 |
| | | gi = 2528462/ug = Mm 20841/len = 178/STRA = for | | | | | | | |
| 8 | 93734_i_at | Cluster Incl D49686: Mus musculus mRNA for Tat binding protein-1, complete cds/cds = (176,1504)/gb = D49686/ | 795.2 | P | 699.3 | P | 627.1 | P | 677.1 |
| | | gi = 3510276/ug = Mm.20946/len = 1569/STRA = for | | | | | | | |
| 8 | 93735_f_at | Cluster Incl D49686: Mus musculus mRNA for Tat binding protein-1, complete cds/cds = (176,1504)/gb = D49686/ | 1645.6 | P | 1634.4 | P | 1043.4 | P | 1510.8 |
| | | gi = 3510276/ug = Mm.20946/len = 1569/STRA = for | | | | | | | |
| 8 | 93760_at | Cluster Incl AI838759 UI-M-AO0-acf-f-02-0-UI s1 Mus musculus cDNA, 3 end/clone = UI-M-AO0-acf-f-02-0-UI/ | 1041.5 | P | 997 | P | 976 | P | 984 |
| | | clone_end = 3/gb = AI838759/gi = 5472972/ug = Mm.21149/len = 460/STRA = for | | | | | | | |
| 8 | 93774_at | Cluster Incl AW049040 UI-M-BH1-amp-h-08-0-UI s1 Mus musculus cDNA, 3 end/clone = UI-M-BH1-amp-h-08-0-UI/ | 704.6 | P | 828.7 | P | 803.3 | P | 723.7 |
| | | clone_end = 3/gb = AW049040/gi = 5909569/ug = Mm.21207/len = 260/STRA = for | | | | | | | |
| 8 | 93789_a_at | Cluster Incl AF038848: Mus musculus transcriptional repressor SIN3B (Sin3B) mRNA, short form, complete cds/ | 880.2 | P | 1410.5 | P | 1048.5 | P | 1247 |
| | | cds = (20,901)/gb = AF038848/gi = 2921546/ug = Mm.2137/len = 1056/STRA = for | | | | | | | |
| 8 | 93823_at | Cluster Incl AI844864: UI-M-AH1-agx-d-05-0-UI.s1 Mus musculus cDNA, 3 end/clone = UI-M-AH1-agx-d-05-0-UI/ | 231.7 | P | 221.6 | P | 175.1 | P | 183.8 |
| | | clone_end = 3/gb = AI844864/gi = 5488735/ug = Mm 21534/len = 527/STRA = for | | | | | | | |
| 8 | 93968_at | Cluster Incl AA870917: vq28a06.r1 Mus musculus cDNA, 5 end/clone = IMAGE-1095538/clone_end = 5/gb = AA870917/ | 983.7 | P | 926.2 | P | 688.3 | P | 935.3 |
| | | gi = 2968382/ug = Mm 21650/len = 454/STRA = for | | | | | | | |
| 8 | 93972_at | Cluster Incl AI8374493 UI-M-AL0-abr-f-12-0-UI.s1 Mus musculus cDNA, 3 end/clone = UI-M-AL0-abr-f-12-0-UI/clone_end = 3 | 1352.5 | P | 1656.1 | P | 1631.8 | P | 1612.9 |
| | | gb = AI83749/gi = 5471706/ug = Mm.21669/len = 341/STRA = for | | | | | | | |
| 8 | 93973_at | Cluster Incl AI854624 UI-M-BH0-ake-e-06-0-UI.a1 Mus musculus cDNA, 3 end/clone = UI-M-BH0-ake-e-06-0-UI/ | 1185.6 | P | 1251.3 | P | 990.2 | P | 1164 |
| | | clone_end = 3/gb = AI854624/gi = 5498530/ug = Mm 21671/len = 474/STRA = for | | | | | | | |
| 8 | 93988_at | Cluster Incl AI836676 UI-M-AP0-abh-g-06-0-UI a1 Mus musculus cDNA, 3 end/clone = UI-M-AP0-abh-g-06-0-UI/ | 1452.8 | P | 1345.5 | P | 1017.7 | P | 1616.8 |
| | | clone_end = 3/gb = AI836676/gi = 5470889/ug = Mm 21728/len = 471/STRA = for | | | | | | | |
| 8 | 94001_at | Cluster Incl U65735 Mus musculus eiav G homolog (MelG) mRNA, complete cds/cds = (0,980)/gb = U65735/gi = 1655900/ | 1307.4 | P | 1116.3 | P | 682.9 | P | 1120.8 |
| | | ug = Mm 21766/len = 981/STRA = for | | | | | | | |
| 8 | 94004_at | Cluster Incl Z19543: M musculus h2-calponin cDNA/cds = (102,1019)/gb = Z51143/ug = Mm 21776/len = 1821/ | 534.7 | P | 626.7 | P | 440.7 | P | 644.3 |
| | | STRA = for | | | | | | | |
| 8 | 94025_at | Cluster Incl AW045339: UI-M-BH1-ako-a-09-0-UI s1 Mus musculus cDNA, 3 end/clone = UI-M-BH1-ako-a-09-0-UI/ | 1294.3 | P | 1541.7 | P | 855.6 | P | 1449.6 |
| | | clone_end = 3/gb = AW045339/gi = 5905868/ug = Mm 21874/len = 430/STRA = for | | | | | | | |
| 8 | 94061_at | Cluster Incl M13018 Cysteine rich Intestinal protein/cds = (61,294)/gb = M13018/gi = 192725/ug = Mm 22049/len = 404/ | 1014.4 | P | 1868 | P | 1249.7 | P | 723.1 |
| | | STRA = for | | | | | | | |
| 8 | 94062_at | Cluster Incl AI847609: UI-M-AP1-agg-c-11-0-UI s1 Mus musculus cDNA, 3 end/clone = UI-M-AP1-agg-c-11-0-UI/ | 2660.6 | P | 2807.2 | P | 2427.6 | P | 2131 |
| | | clone_end = 3/gb = AI847609/gi = 5491515/ug = Mm 2206/len = 356/STRA = for | | | | | | | |
| 8 | 94078_at | Cluster Incl AW046239: UI-M-BH1-ala-b-12-0-UI s1 Mus musculus cDNA, 3 end/clone = UI-M-BH1-ala-b-12-0-UI/ | 2066.4 | P | 2492.8 | P | 1852.7 | P | 2239.8 |
| | | clone_end = 3/gb = AW046239/gi = 5906768/ug = Mm 22138/len = 363/STRA = for | | | | | | | |
| 8 | 94219_at | Cluster Incl AI853269: UI-M-BH0-ajl-c-11-0-UI s1 Mus musculus cDNA, 3 end/clone = UI-M-BH0-ajl-c-11-0-UI/clone_end = 3/ | 1390.3 | P | 1476 | P | 1348.1 | P | 1569 |
| | | gb = AI853269/gi = 5479175/ug = Mm 22233/len = 375/STRA = for | | | | | | | |
| 8 | 94263_f_at | Cluster Incl D85570 Proteasome (prosome, macropain) subunit, beta type 7/cds = (20,853)/gb = D85570/gi = 2547069/ | 1913.9 | P | 2273.5 | P | 1907.1 | P | 2176.1 |
| | | ug = Mm 2246/len = 991/STRA = for | | | | | | | |
| 8 | 94821_at | Cluster Incl AW123880 UI-M-BH2.1-app-e-07-0-UI.s1 Mus musculus cDNA, 3 end/clone = UI-M-BH2.1-app-e-07-0-UI/ | 1324.2 | P | 1268.5 | P | 1292.8 | P | 1598.9 |
| | | clone_end = 3/gb = W123880/gi = 6099410/ug = Mm 22718/len = 448/STRA = for | | | | | | | |
| 8 | 94841_at | Cluster Incl AW148997 UI-M-BH1-amp-c-10-0-UI s1 Mus musculus cDNA, 3 end/clone = UI-M-BH1-amp-c-10-0-UI/ | 1185.9 | P | 1134.1 | P | 946.3 | P | 1208.5 |
| | | clone_end = 3/gb = AW048997/gi = 5909526/ug = Mm 2287/len = 486/STRA = for | | | | | | | |
| 8 | 94875_at | Cluster Incl AI838915 UI-M-AO0-ace-a-11-0-UI s1 Mus musculus cDNA, 3 end/clone = UI-M-AO0-ace-a-11-0-UI/ | 560.5 | P | 586 | P | 390.5 | P | 631.4 |
| | | clone_end = 3/gb = AI838915/gi = 5473128/ug = Mm 23825/len = 398/STRA = for | | | | | | | |
| 8 | 94876_f_at | Cluster Incl AI849207: UI-M-AJ1-agz-a-02-0-UI s1 Mus musculus cDNA, 3 end/clone = UI-M-AJ1-agz-a-02-0-UI/ | 803.4 | P | 828 | P | 772.5 | P | 893.5 |
| | | clone_end = 3/gb = AI849207/gi = 5493113/ug = Mm 23836/len = 449/STRA = for | | | | | | | |
| 8 | 94897_at | Cluster Incl D87896 Glutathione peroxidase 4/cds = (144,737)/gb = D87896/gi = 2522258/ug = Mm 2400/len = 958/STRA = for | 687.6 | P | 646.7 | P | 605.2 | P | 695 |
| 8 | 94907_f_at | Cluster Incl AW045632: UI-M-BH1-alg-e-06-0-UI s1 Mus musculus cDNA, 3 end/clone = UI-M-BH1-alg-e-06-0-UI/ | 1015.8 | P | 1429.8 | P | 1024.8 | P | 1240.3 |
| | | clone _end = 3/gb = AW045632/gi = 5906161/ug = Mm 24091/len = 248/STRA = for | | | | | | | |
| 8 | 94909_at | Cluster Incl AW046919: UI-M-BH1-akl-g-03-0-UI s1 Mus musculus cDNA, 3 end/clone = UI-M-BH1-akl-g-03-0-UI/ | 454.7 | P | 506.6 | P | 397.8 | P | 429 |
| | | clone _end = 3/gb = AW046919/gi = 5907448/ug = Mm 24104/len = 440/STRA = for | | | | | | | |
| 8 | 94912_at | Cluster Incl AI835592: UI-M-AI0-app-a-05-0-UI.s1 Mus musculus cDNA, 3 end/clone = UI-M-AI0-aap-a-05-0-UI/ | 750.9 | P | 781 | P | 748.7 | P | 694.7 |
| | | clone_end = 3/gb = AI835592/gi = 5469805/ug = Mm 24108/len = 229/STRA = for | | | | | | | |
| 8 | 94913_at | Cluster Incl U65313 Mus musculus ras-GTPase-activating protein SH3-domain binding protein (G3BP) mRNA, complete | 516.6 | P | 550.2 | P | 466.5 | P | 542.9 |
| | | cds/cds = (164,1612)/gb = U65313/gi = 2961438/ug = Mm.2411/len = 1800/STRA = for | | | | | | | |
| 8 | 95409_at | Cluster Incl AW047671: UI-M-BH1-alo-c-06-0-UI s1 Mus musculus cDNA, 3 end/clone = UI-M-BH1-alo-c-06-0-UI/ | 664.2 | P | 849 | P | 460.2 | P | 745.9 |
| | | clone_end = 3/gb = AW047671/gi = 5908200/ug = Mm 24228/len = 339/STRA = for | | | | | | | |
| 8 | 95424_at | Cluster Incl AW120986: UI-M-BH2 3-aod-f-01-0-UI s1 Mus musculus cDNA, 3 end/clone = UI-M-BH2.3-aod-f-01-0-UI/ | 3138.8 | P | 3944 | P | 3337.6 | P | 3375.6 |
| | | clone_end = 3/gb = AW120986/gi = 609631/ug = Mm 24433/len = 498/STRA = for | | | | | | | |
| 8 | 95441_at | Cluster Incl AB201122: Mus musculus Tim23 mRNA for translocase of inner mitochondrial membrane, complete cds/ | 2577.5 | P | 2677.3 | P | 2402.1 | P | 2576.8 |
| | | cds = (123,752)/gb = AB021122/gi = 4996327/ug = Mm 24565/len = 1142/STRA = for | | | | | | | |
| 8 | 95462_at | Cluster Incl AW060951 UI-M-BH1-anv-f-08-0-UI s1 Mus musculus cDNA, 3 end/clone = UI-M-BH1-anv-f-08-0-UI/ | 1265.3 | P | 1304.1 | P | 997.4 | P | 1178.5 |
| | | clone_end = 3/gb = AW060951/gi = 6008702/ug = Mm 24745/len = 228/STRA = for | | | | | | | |
| 8 | 95517_i_at | Cluster Incl AI846338: UI-M-AN1-afa-c-01-0-UI.s1 Mus musculus cDNA, 3 end/clone = UI-M-AN1-afa-c-10-0-UI/ | 685.6 | P | 983.4 | P | 843.2 | P | 770.5 |
| | | clone_end = 3/gb = AI846338/gi = 5490244/ug = Mm 25308/len = 377/STRA = for | | | | | | | |
| 8 | 96008_at | Cluster Incl U81052: Mus musculus Defender against Apoptotic Death (Dad1) gene/cds = (25,366)/gb = U81052/gi = 1905978/ | 1491.2 | P | 1645.5 | P | 1283.7 | P | 1608.5 |
| | | ug = Mm 2547/len = 406/STRA = for | | | | | | | |
| 8 | 96611_at | Cluster Incl AI843074 UI-M-AK1-aes-s-11-0-UI s1 Mus musculus cDNA, 3 end/clone = UI-M-AK1-aes-a-11-0-UI/ | 1076.9 | P | 1370.3 | P | 835 | P | 925 |
| | | clone_end = 3/gb = AI843074/gi = 5477287/ug = Mm 27087/len = 253/STRA = for | | | | | | | |
| 8 | 96666_at | Cluster Incl U57692 N-terminal Asn amidase/cds = (33,965)/gb = U57692/gi = 1373366/ug = Mm 27378/len = 1101/STRA = for | 33.3 | P | 205.8 | P | 58.5 | P | 87.9 |
| 8 | 96686_i_at | Cluster Incl AI853864: UI-M-BH0-ajr-f-08-0-UI s1 Mus musculus cDNA, 3 end/clone = UI-M-BH0-ajr-f-08-0-UI/clone_end = 3/ | 2080.1 | P | 2299.8 | P | 1688.2 | P | 1781.5 |
| | | gb = AI853864/gi = 5497770 ug = Mm.27477/len = 461/STRA = for | | | | | | | |
| 8 | 96687_f_at | Cluster Incl AI853864 UI-M-BH0-ajr-f-08-0-UI s1 Mus musculus cDNA, 3 end/clone = UI-M-BH0-ajr-f-08-0-UI/clone_end = 3/ | 939.2 | P | 1056.2 | P | 849.9 | P | 904.9 |
| | | gb = AI853864/gi = 5497770 ug = Mm 27477/len = 461/STRA = for | | | | | | | |
| 8 | 97203_at | Cluster Incl X61399: Mouse F52 mRNA for a novel protein/cds = UNKNOWN/gb = X61399/gi = 50943/ug = Mm 2769/ | 1741.4 | P | 1381.5 | P | 1497.9 | P | 1590.5 |
| | | len = 1584/STRA = for | | | | | | | |
| 8 | 97204_s_at | Cluster Incl AI850983: UI-M-BH0-akd-h-08-0-UI s1 Mus musculus cDNA, 3 end/clone = UI-M-BH0-akd-h-08-0-UI/ | 343.5 | P | 324.1 | P | 247.6 | P | 294.4 |
| | | clone_end = 3/gb = AI850983/gi = 5494889/ug = Mm 27692/len = 265/STRA = for | | | | | | | |
| 8 | 97207_f_at | Cluster Incl AI875934: uj64h06 x1 Mus musculus cDNA, 3 end/clone = IMAGE-1924763/clone_end = 3/gb = AI675934/ | 2471.9 | P | 2768.3 | P | 2176.7 | P | 2171.9 |
| | | gi = 5549983/ug = Mm 27700/len = 330/STRA = for | | | | | | | |
| 8 | 97248_at | Cluster Incl X61431: M. musculus mRNA for diazepam-binding inhibitor/cds = (62,325)/gb = X61431/gi = 50712/ug = Mm.2785/ | 2217.5 | P | 2657.5 | P | 2527.8 | P | 2316.9 |
| | | len = 390/STRA = for | | | | | | | |
| 8 | 97911_at | Cluster Incl AI835950-UI-M-AI0-aao-f-11-0-UI.s1 Mus musculus cDNA. 3 end/clone = UI-M-AI0-aao-f-11-0-UI/clone_end = 3/ | 644.2 | P | 743.2 | P | 609.6 | P | 552.3 |
| | | gb = AI835950/gi = 5470163/ug = Mm 28482/len = 380/STRA = for | | | | | | | |
| 8 | 98875_at | Cluster Incl AW121160: UI-M-BH2 3-aoh-g-08-0-UI s1 Mus musculus cDNA, 3 end/clone = UI-M-BH2 3-aoh-g-08-0-UI/ | 155.5 | P | 189.2 | P | 72.8 | P | 157.3 |
| | | clone_end = 3/gb = AW121160/gi = 6096493/ug = Mm 28492/len = 139/STRA = for | | | | | | | |
| 8 | 94494 _at | Cluster Incl AI840856 UI-M-AH0-adb-g-11-0-UI s1 Mus musculus cDNA, 3 end/clone = UI-M-AH0-adb-g-11-0-UI/ | 367.9 | P | 441.2 | P | 296.3 | P | 469.4 |
| | | clone_end = 3/gb = AI840856/gi = 5475069/ug = Mm 28922/len = 274/STRA = for | | | | | | | |
| 8 | 94506 _at | Cluster Incl AI853113 UI-M-BH0-ajf-d-10-0-UI s1 Mus musculus cDNA, 3 end/clone = UI-M-BH0-ajf-d-10-0-UI/ | 554.7 | P | 453.9 | P | 384.5 | P | 511.2 |
| | | clone_end = 3/gb = AI853113/gi = 5497019/ug = Mm.28961/len = 205/STRA = for | | | | | | | |
| 8 | 94522 _at | Cluster Incl AF098508 Mus musculus dynaclin light chain (P24) mRNA, complete cds/cds = (12,572)/gb = AF098508/ | 726.9 | P | 779.2 | P | 653.2 | P | 674.7 |
| | | gi = 4323147/ug = Mm.29025/len = 819/STRA = for | | | | | | | |
| 8 | 94550 _at | Cluster Incl AW121324 UI-M-BH2.2-aom-a-06-0-UI s1 Mus musculus cDNA, 3 end/clone = UI-M-BH2.2-aom-a-06-0-UI/ | 484.8 | P | 544 | P | 588.4 | P | 516.8 |
| | | clone_end = 3/gb = AW121324/gi = 6096657/ug = Mm 29079/len = 541/STRA = for | | | | | | | |
| 8 | 95042 _at | Cluster Incl AI839770 UI-M-AN0-acq-f-01-0-UI s1 Mus musculus cDNA, 3 end/clone = UI-M-AN0-acq-f-01-0-UI/ | 632.6 | P | 622.5 | P | 545.5 | P | 614.1 |
| | | clone_end = 3/gb = AI839770/gi = 5474023/ug = Mm 29118/len = 148/STRA = for | | | | | | | |
| 8 | 95066 _at | Cluster Incl U67611 Mouse transaidolase gene mRNA, complete cds/cds = (5,1018)/gb = U67611/gi = 2589165/ | 451.4 | P | 553.4 | P | 408.8 | P | 445.4 |
| | | ug = Mm 29132/len = 1155/STRA = for | | | | | | | |
| 8 | 95091_at | Cluster Incl AI839895.UI-M-AH0-acw-d-02-0-UI s1 Mus musculus cDNA, 3 end/clone = UI-M-AH0-acw-d-02-0-UI/ | 842.4 | P | 1030.3 | P | 605.5 | P | 909.6 |
| | | clone_end = 3/gb = AI839895/gi = 5474108/ug = Mm 29296/len = 446/STRA = for | | | | | | | |
| 8 | 95097 _at | Cluster Incl AI851227: UI-M-BH0-ajx-h-07-0-UI s1 Mus musculus cDNA, 3 end/clone = UI-M-BH0-ajx-h-07-0-UI/ | 686.3 | P | 734.6 | P | 579.6 | P | 706.4 |
| | | clone_end = 3/gb = AI851227/gi = 5495133/ug = Mm 29317/len = 354/STRA = for | | | | | | | |
| 8 | 95131_f_at | Cluster Incl AI852592: UI-M-BH0-aiu-b-11-0-UI s1 Mus musculus cDNA, 3 end/clone = UI-M-BH0-aiu-b-11-0-UI/ | 2279.4 | P | 2637.4 | P | 2147.8 | P | 2202.5 |
| | | clone_end = 3/gb = AI852592/gi = 5496498/ug = Mm 29415/len = 392/STRA = rev | | | | | | | |
| 8 | 95137 _at | Cluster Incl AI852985: UI-M-BH0-ajn-a-04-0-UI s1 Mus musculus cDNA, 3 end/clone = UI-M-BH0-ajn-a-04-0-UI/ | 755.7 | P | 890.1 | P | 588.5 | P | 798.6 |
| | | clone_end = 3/gb = AI852985/gi = 5496891/ug = Mm 29431/len = 428/STRA = for | | | | | | | |
| 8 | 95147 _at | Clusler Incl AI843795: UI-M-AN1-afc-e-05-0-UI.s1 Mus musculus cDNA, 3 end/clone = UI-M-AN1-afc-e-05-0-UI/ | 1925.6 | P | 2195.1 | P | 1874 | P | 1996.6 |
| | | clone_end = 3/gb = AI843795/gi = 5478008/ug = Mm 29456/len = 462/STRA = for | | | | | | | |
| 8 | 95149_at | Cluster Incl AW121088: UI-M-BH2 3-aof-h-04-0-UI s1 Mus musculus cDNA, 3 end/clone = UI-M-BH2.3-aof-h-04-0-UI/ | 798.7 | P | 979.9 | P | 826.2 | P | 722.7 |
| | | clone_end = 3/gb = AW121088/gi = 6096421/ug = Mm 29473/len = 505/STRA = for | | | | | | | |
| 8 | 95659_at | Cluster Incl AF041476 Mus musculus BAF53a (Baf53a) mRNA, complete cds/cds = (130,1419)/gb = AF041476/gi = 4001804/ | 528.5 | P | 549.5 | P | 424.6 | P | 437.2 |
| | | ug = Mm 29546/len = 1770/STRA = for | | | | | | | |
| 8 | 95690_at | Cluster Incl AW047363 UI-M-BH1-ame-c-09-0-UI s1 Mus musculus cDNA, 3 end/clone = UI-M-BH1-ame-c-09-0-UI/ | 442.4 | P | 683.3 | P | 405.2 | P | 562.3 |
| | | clone_end = 3/gb = AW047363/gi = 5907892/ug = Mm 29651/len = 398/STRA = for | | | | | | | |
| 8 | 95696_at | Cluster Incl AI840882: UI-M-AM0-adm-b-04-0-UI.s1 Mus musculus cDNA, 3 end/clone = UI-M-AM0-adm-b-04-0-UI/ | 2122.8 | P | 1961 | P | 1660.3 | P | 1766.6 |
| | | clone_end = 3/gb = AI840882/gi = 5475095/ug = Mm 29675/len = 400/STRA = for | | | | | | | |
| 8 | 95700_f_at | Cluster Incl AI848094 UI-M-AP1-agk-d-05-0-UI s1 Mus musculus cDNA, 3 end/clone = UI-M-AP1-agk-d-05-0-UI/ | 443.7 | P | 343.4 | P | 347.1 | P | 383.2 |
| | | clone_end = 3/gb = AI848094/gi = 5492000/ug = Mm 29685/len = 399/STRA = for | | | | | | | |
| 8 | 95703_at | Cluster Incl AB024303 Mus musculus mRNA similar to human Sua1, complete cds/cds = (34,1086)/gb = AB024303/ | 907.4 | P | 744.5 | P | 685.6 | P | 730 |
| | | gi = 5689241/ug = Mm 29698/len = 1870/STRA = for | | | | | | | |
| 8 | 95707_at | Cluster Incl AA615853 Vo94104.r1 Mus musculus cDNA, 5 end/clone = IMAGE-1066783/clone_end = 5/gb = AA615853/ | 867.4 | P | 950.8 | P | 558.9 | P | 666 |
| | | gi = 2503081/ug = Mm 29700/len = 534/STRA = for | | | | | | | |
| 8 | 95714_at | Cluster Incl AI226264: uf06a12y1 Mus musculus cDNA, 5 end/clone = IMAGE-1510558/clone_end = 5/gb = AI226264/ | 720.9 | P | 897 | P | 699.3 | P | 764.3 |
| | | gi = 3809317/ug = Mm 29712/len = 465/STRA = for | | | | | | | |
| 8 | 95716_at | Cluster Incl AW125041: UI-M-BH2 1-apx-b-01-0-UI s1 Mus musculus cDNA, 3 end/clone = UI-M-BH2 1-apx-b-01-0-UI/ | 2782.7 | P | 2934.5 | P | 2752.9 | P | 2827.2 |
| | | clone_end = 3/gb = AW125041/gi = 6100571/ug = Mm 29717/len = 227/STRA = for | | | | | | | |
| 8 | 95718_f_at | Cluster Incl AW060158 UI-M-BH1-amu-b-10-0-UI s1 Mus musculus cDNA, 3 end/clone = UI-M-BH1-amu-b-10-0-UI/ | 3094.4 | P | 3112.3 | P | 2746.2 | P | 3113.2 |
| | | clone_end = 3/gb = AW060158/gi = 6007924/ug = Mm 29722/len = 360/STRA = for | | | | | | | |
| 8 | 95736_at | Cluster Incl AI847546: UI-M-AM1-afv-f-01-0-UI s1 Mus musculus cDNA, 3 end/clone = UI-M-AM1-afv-f-01-0-UI/ | 756.6 | P | 799.3 | P | 777.7 | P | 777.6 |
| | | clone_end = 3/gb = AI847546/gi = 5491452/ug = Mm 29749/len = 452/STRA = for | | | | | | | |
| 8 | 95742_at | Cluster Incl AW045451: UI-M-BH1-akr-g-10-0-UI s1 Mus musculus cDNA, 3 end/clone = UI-M-BH1-akr-g-10-0-UI/ | 635.7 | P | 647.7 | P | 451.1 | P | 614.3 |
| | | clone_end = 3/gb = AW045451/gi = 5906020/ug = Mm 29760/len = 340/STRA = for | | | | | | | |
| 8 | 95760_at | Cluster Incl AI848730: UI-M-AJ1-ahf-d-04-0-UI.s1 Mus musculus cDNA, 3 end/clone = UI-M-AJ1-ahf-d-04-0-UI/ | 376.7 | P | 508.2 | P | 375.5 | P | 346.6 |
| | | clone_end = 3/gb = AI848730/gi = 5492636/ug = Mm 29801/len = 477/STRA = for | | | | | | | |
| 8 | 96249_at | Cluster Incl AW122105: UI-M-BH2 2-aoo-h-12-0-UI.s1 Mus musculus cDNA, 3 end/clone = UI-M-BH2.2-aoo-h-12-0-UI/ | 972.4 | P | 1022 | P | 783.7 | P | 849.8 |
| | | clone_end = 3/gb = AW122105/gi = 6097518/ug = Mm.29812/len = 357/STRA = rev | | | | | | | |
| 8 | 96258_at | Cluster Incl AI843448. UI-M-AQ1-aec-e-01-0-UI s1 Mus musculus cDNA, 3 end/clone = UI-M-AQ1-aec-e-01-0-UI/ | 2323.3 | P | 2031.3 | P | 1449.3 | P | 1773.4 |
| | | clone_end = 3/gb = AI843448/gi = 5477661/ug = Mm 29823/len = 382/STRA = for | | | | | | | |
| 8 | 96276_r_at | Cluster Incl AI843327: UI-M-AQ1-adz-a-05-0-UI s1 Mus musculus cDNA, 3 end/clone = UI-M-AQ1-adz-a-05-0-UI/ | 333.1 | P | 367.7 | P | 285.9 | P | 364.1 |
| | | clone_end = 3/gb = AI843327/gi = 5477540/ug = Mm 2986/len = 339/STRA = for | | | | | | | |
| 8 | 96291_f_at | Cluster Incl AI835847 UI-M-AI0-aat-b-07-0-UI s1 Mus musculus cDNA, 3 end/clone = UI-M-AI0-aat-b-07-0-UI/clone_end = 3/ | 1368.3 | P | 1464.1 | P | 1278.4 | P | 1221.3 |
| | | gb = AI835847/gi = 5470060/ug = Mm 29897/len = 333/STRA = for | | | | | | | |
| 8 | 96292_r_at | Cluster Incl AI835847 UI-M-AI0-aat-b-07-0-UI s1 Mus musculus cDNA, 3 end/clone = UI-M-AI0-aat-b-07-0-UI/clone_end = 3/ | 1839.8 | P | 2255.6 | P | 1946.5 | P | 1866.1 |
| | | gb = AI835847/gi = 5470060/ug = Mm 29897/len = 333/STRA = for | | | | | | | |
| 8 | 96305_at | Cluster Incl AI845854.UI-M-AO1-aeg-e-10-0-UI s1 Mus musculus cDNA, 3 end/clone = UI-M-AO1-aeg-e-10-0-UI/ | 536.3 | P | 510 | P | 439.7 | P | 487.4 |
| | | clone_end = 3/gb = AI845854/gi = 5489760/ug = Mm 29916/len = 441/STRA = for | | | | | | | |
| 8 | 96321_at | Cluster Incl AI846396: UI-M-AN1-afa-h-08-0-UI s1 Mus musculus cDNA, 3 end/clone = UI-M-AN1-afa-h-08-0-UI/ | 1743.4 | P | 1713.8 | P | 1574.5 | P | 1543.7 |
| | | clone_end = 3/gb = AI846396/gi = 5490302/ug = Mm 29939/len = 380/STRA = for | | | | | | | |
| 8 | 96329_at | Cluster Incl AI837793.UI-M-AK0-adl-e-09-0-UI s1 Mus musculus cDNA, 3 end/clone = UI-M-AK0-adl-e-09-0-UI/ | 527.8 | P | 512.3 | P | 450.8 | P | 498.6 |
| | | clone_end = 3/gb = AI837793/gi = 5472006/ug = Mm 29965/len = 480/STRA = for | | | | | | | |
| 8 | 96341_at | Cluster Incl AI849280 UI-M-AJ1-agz-h-05-0-UI.s1 Mus musculus cDNA, 3 end/clone = UI-M-AJ1-agz-h-05-0-UI/ | 371 | P | 358.9 | P | 267.5 | P | 378.1 |
| | | clone_end = 3/gb = AI849280/gi = 5493186/ug = Mm 29989/len = 520/STRA = for | | | | | | | |
| 8 | 96342_at | Cluster Incl AI846320: UI-M-AN1 -afa-a-04-0-U1.s1 Mus musculus cDNA, 3 end/clone = UI-M-AN1-afa-a-04-0-UI/ | 454 | P | 393.1 | P | 354.3 | P | 458.9 |
| | | clone_end = 3/gb = AI846320/gi = 5490226/ug = Mm.29990/len = 281/STRA = for | | | | | | | |
| 8 | 96360_at | Cluster Incl AW125498: UI-M-BH2.2-aqm-d-11-0-UI.s1 Mus musculus cDNA, 3 end/clone = UI-M-BH2.2-aqm-d-11-0-UI/ | 1863 | P | 2320.5 | P | 2316.7 | P | 2375.8 |
| | | clone_end = 3/gb = AW125498/gi = 6101028/ug = Mm 30016/len = 389/STRA = for | | | | | | | |
| 8 | 96855_at | Cluster Incl AI839946: UI-M-AHO-acs-a-04-0-UI.s1 Mus musculus cDNA, 3 end/clone = UI-M-AH0-acs-a-04-0-UI/ | 883.7 | P | 876.4 | P | 859.3 | P | 892.8 |
| | | clone_end = 3/gb = AI839946/gi = 5474159/ug = Mm 30043/len = 255/STRA = for | | | | | | | |
| 8 | 96883_at | Cluster Incl U70733 Mus musculus translation initiation factor eIF3-p44 mRNA, complete cds/cds = (17,979)/gb = U70733/ | 1244.6 | P | 1413.3 | P | 1248.2 | P | 1295.9 |
| | | gi = 4097872/ug = Mm 30080/len = 1025/STRA = for | | | | | | | |
| 8 | 96952_at | Cluster Incl AW121552 UI-M-BH2 2-aop-f-02-0-UI s1 Mus musculus cDNA, 3 end/clone = UI-M-BH2 2-aop-f-02-0-UI/ | 2102.8 | P | 2480.1 | P | 2097 | P | 2413 |
| | | clone_end = 3/gb = AW121552/gi = 6096885/ug = Mm 30210/len = 521/STRA = for | | | | | | | |
| 8 | 97443_at | Cluster Incl AI850850 UI-M-BH0-ajt-c-05-0-UI s1 Mus musculus cDNA, 3 end/clone = UI-M-BH0-ajt-c-05-0-UI/clone_end = 3/ | 1404.6 | P | 1711.9 | P | 1361 | P | 1827.6 |
| | | gb = AI850850/gi = 5494756/ug = Mm 30240/len = 321/STRA = for | | | | | | | |
| 8 | 97528_at | Cluster Incl AF022811 Mus musculus cornichon mRNA, complete cds/cds = (85,519)/gb = AF022811/gi = 3114215/ | 779.5 | P | 825.1 | P | 813.1 | P | 755.1 |
| | | ug = Mm 3261/len = 1388/STRA = for | | | | | | | |
| 8 | 98104_at | Cluster Incl AI842889 UI-M-AN1-afb-g-01-0-UI s1 Mus musculus cDNA, 3 end/clone = UI-M-AN1-alb-g-01-0-UI/ | 960 | P | 842.8 | P | 873.4 | P | 973.2 |
| | | clone_end = 3/gb = AI842889/gi = 5477102/ug = Mm 3479/len = 304/STRA = for | | | | | | | |
| 8 | 98545_at | Cluster Incl X78683 B-cell receptor-associated protein 37/cds = (107,1003)/gb = X78683/gi = 541733/ug = Mm 36241/ | 1613.9 | P | 1786.3 | P | 1848.5 | P | 1943.7 |
| | | len = 1287/STRA = for | | | | | | | |
| 8 | 98575_at | Cluster Incl X13135 Fatty acid synthase/cds = (0,2516)/gb = X13135/gi = 50947/ug = Mm 3760/len = 3295/STRA = for | 1003.4 | P | 1944.6 | P | 651.4 | P | 1034 |
| 8 | 98618_at | Cluster Incl AA624336 vn01g09.r1 Mus musculus cDNA, 5 end/clone = IMAGE-1006528/clone_end = 5/gb = AA624336/ | 626.5 | P | 583.9 | P | 325.3 | P | 528 |
| | | gi = 2528212/ug = Mm 3856/len = 474/STRA = for | | | | | | | |
| 8 | 98631_g_at | Cluster Incl AW106745 um33b04 y1 Mus musculus cDNA, 5 end/clone = IMAGE-2236303/clone_end = 5/gb = AW106745/ | 348.4 | P | 340.7 | P | 307 | P | 308.3 |
| | | gi = 6077545/ug = Mm 38792/len = 600/STRA = for | | | | | | | |
| 8 | 99104_at | Cluster Incl U49915.Mus musculus adipocyte specific protein adipoQ (adipoQ) mRNA, complete cds/cds = (16,759)/ | 3857.7 | P | 4540.9 | P | 3299.3 | P | 3607.9 |
| | | gb = U49915/gi = 1399497/ug = Mm 3969/len = 1152/STRA = for | | | | | | | |
| 8 | 99108_s_at | Cluster Incl U15012 Growth hormone receptor/cds = (0,430)/gb = U15012/gi = 666863/ug = Mm 3986/len = 513/STRA = for | 342.4 | P | 389.3 | P | 242.2 | P | 271.1 |
| 8 | 99140_at | Cluster Incl AW 124920 UI-M-BH2 1-apu-f-09-0-UI s1 Mus musculus cDNA, 3 end/clone = UI-M-BH2 1-apu-f-09-0-UI/ | 359.7 | P | 382.8 | P | 357.7 | P | 357.5 |
| | | clone_end = 3/gb = AW124920/gi = 6100450/ug = Mm 41390/len = 426/STRA = for | | | | | | | |
| 8 | 99150_at | Cluster Incl AI844357.UI-M-AL1-ahp-a-10-0-UI.s1 Mus musculus cDNA, 3 end/clone = UI-M-AL1-ahp-a-10-0-UI/ | 525.7 | P | 627.2 | P | 467.5 | P | 496.9 |
| | | clone_end = 3/gb = AI844357/gi = 5488263/ug = Mm 41541/len = 413/STRA = for | | | | | | | |
| 8 | 99564_at | Cluster Incl D87908 Mus musculus mRNA for nuclear protein np95, complete cds/cds = (148,2496)/gb = D87908/ | 464.9 | P | 617.2 | P | 352.9 | P | 419.2 |
| | | gi = 4220589/ug = Mm 42196/len = 3488/STRA = for | | | | | | | |
| 8 | 99581_at | Cluster Incl U60001 Mus musculus protein kinase C inhibitor (mPKCI) mRNA, complete cds/cds = (22,402)/gb = U60001/ | 3311.9 | P | 3890.5 | P | 3300.7 | P | 3451.4 |
| | | gi = 1519045/ug = Mm 425/len = 544/STRA = for | | | | | | | |
| 8 | 99597_at | Cluster Incl AI841629 UI-M-AO0-acg-c-01-0-UI s1 Mus musculus cDNA, 3 end/clone = UI-M-AO0-acg-c-01-0-UI/ | 529.4 | P | 626.9 | P | 700.8 | P | 712.9 |
| | | clone_end = 3/gb = AI841629/gi = 5475842/ug = Mm 4284/len = 436/STRA = for | | | | | | | |
| 8 | 100079_at | Cluster Incl AI845556 UI-M-AO1-aek-h-05-0-UI.s1 Mus musculus cDNA, 3 end/clone = UI-M-AO1-aek-h-05-0-UI/ | 1415.4 | P | 1837 | P | 1402.1 | P | 1776.9 |
| | | clone_end = 3/gb = AI845556/gi = 5489462/ug = Mm 4537/len = 428/STRA = for | | | | | | | |
| 8 | 100081_at | Cluster Incl U27830 Mus musculus mSTI1 mRNA, complete cds/cds = (55,1686)/gb = U27830/gi = 881484/ug = Mm 4540/ | 768.5 | P | 556.4 | P | 434.9 | P | 612.7 |
| | | len = 2080/STRA = for | | | | | | | |
| 8 | 100101_at | Cluster Incl L15447.Mouse small nuclear RNA (Rnu1a-1) mRNA, complete cds/cds = (99,962)/gb = L15447/gi = 349003/ | 692.6 | P | 591.4 | P | 645.4 | P | 653.1 |
| | | ug = Mm 4633/len = 1176/STRA = for | | | | | | | |
| 8 | 100527_at | Cluster Incl AW124744 UI-M-BH2.1-aqa-d-10-0-UI s1 Mus musculus cDNA, 3 end/clone = UI-M-BH2 1-aqa-d-10-0-UI/ | 546.4 | P | 428 | P | 419.4 | P | 490.6 |
| | | clone_end = 3/gb = AW124744/gi = 6100274/ug = Mm.52/len = 485/STRA = for | | | | | | | |
| 8 | 100575_at | Cluster Incl AI841645 UI-M-AO0-acg-d-06-0-UI s1 Mus musculus cDNA, 3 end/clone = UI-M-AO0-acg-d-06-0-UI/ | 422.9 | P | 379.5 | P | 285.5 | P | 321.5 |
| | | clone_end = 3/gb = AI841645/gi = 5475858/ug = Mm 5934/len = 256/STRA = for | | | | | | | |
| 8 | 101016_at | Cluster Incl D87898 Mus musculus mRNA for ARF1, complete cds/cds = (78,623)/gb = D87898/gi = 1565206/ug = Mm.6836/ | 4718.2 | P | 4772.6 | P | 4521.1 | P | 4948.6 |
| | | len = 1032/STRA = for | | | | | | | |
| 8 | 101045_at | Cluster Incl U96116: Mus musculus amyloid beta-peptide binding protein (ERAB) mRNA, complete cds/cds = (104,898)/ | 814.5 | P | 813.8 | P | 633.9 | P | 789 |
| | | gb = U96116/gi = 2078283/ug = Mm.6994/len = 1029/STRA = for | | | | | | | |
| 8 | 101085_at | Cluster Incl AI854257 UI-M-BH0-ajs-b-02-0-UI s1 Mus musculus cDNA, 3 end/clone = UI-M-BH0-ajs-b-02-0-UI/ | 900.7 | P | 784.6 | P | 698.2 | P | 745.8 |
| | | clone_end = 3/gb = AI854257/gi = 5498163/ug = Mm.7328/len = 511/STRA = for | | | | | | | |
| 8 | 101112_g_at | Cluster Incl AF014371: Mus musculus Rho family GTPase (ArhA) mRNA, complete cds/cds = (134,715)/gb = AF014371/ | 1120.4 | P | 1044.4 | P | 1168.5 | P | 1408.2 |
| | | gi = 3237319/ug = Mm.757/len = 815/STRA = for | | | | | | | |
| 8 | 101510_at | Cluster Incl AB007136 Protease (prosome, macropain) 28 subunit, alpha/cds = (81,830)/gb = AB007136/gi = 3219281/ | 583.3 | P | 700.5 | P | 499.7 | P | 701.8 |
| | | ug = Mm 830/len = 977/STRA = for | | | | | | | |
| 8 | 101525_at | Cluster Incl AI848871 UI-M-AJ1-ahb-f-06-0-UI.s1 Mus musculus cDNA 3 end/clone = UI-M-AJ1-ahb-f-06-0-UI/ | 1099.9 | P | 1200 | P | 900.2 | P | 989 |
| | | clone_end = 3/gb = AI848871/gi = 5492777/ug = Mm.8688/len = 353/STRA = for | | | | | | | |
| 8 | 101558_s_at | Cluster Incl AB003304 Proteasome beta type subunit 5/cds = (7,801)/gb = AB003304/gi = 2897805/ug = Mm 8911/len = 874/ | 1463.2 | P | 1449.8 | P | 1161.8 | P | 1619.4 |
| | | STRA = for | | | | | | | |
| 8 | 101954_at | Cluster Incl U70494 Mus musculus histone H2A Z (H2A Z) mRNA, complete cds/cds = (95,481)/gb = U70494/gi = 1575712/ | 4060.6 | P | 3826.8 | P | 2973.2 | P | 3245.2 |
| | | ug = Mm 916/len = 989/STRA = for | | | | | | | |
| 8 | 101961_at | Cluster Incl U67327.Mus musculus WD40-repeat type I transmembrane protein A72.5 mRNA, complete cds/ | 1567.6 | P | 1551.6 | P | 1182.7 | P | 1505.8 |
| | | cds = (291,1031)/gb = U67327/gi = 1762983/ug = Mm 927/len = 1336/STRA = for | | | | | | | |
| 8 | 101992_at | Cluster Incl U13393: Mus musculus delta proteasome subunit mRNA, complete cds/cds = (69,677)/gb = U13393/gi = 984937/ | 1147.1 | P | 1336.8 | P | 841.8 | P | 1162.6 |
| | | ug = Mm 98/len = 721/STRA = for | | | | | | | |
| 8 | 102047_at | Cluster Incl AF043326 Mus musculus N-myristoyltransferase 1 mRNA complete cds/cds = (22,1512)/gb = AF043326/ | 658.2 | P | 605.7 | P | 621.5 | P | 876.5 |
| | | gi = 3005066/ug = Mm 10265/len = 1548/STRA = for | | | | | | | |
| 8 | 102821_s_at | Cluster Incl L32752 Mouse (clone M2) GTPase (Ran) mRNA, complete cds/cds = (37,687)/gb = L32752/gi = 727168/ | 3551.9 | P | 3225.4 | P | 2141.4 | P | 3206.9 |
| | | ug = Mm 14426/len = 970/STRA = for | | | | | | | |
| 8 | 103350_at | Cluster Incl M64641: Moloney leukemia virus 34/cds = (78,1043)/gb = M64641/gi = 199770/ug = Mm.18347/len = 1547/ | 1044.3 | P | 994.8 | P | 900.7 | P | 1009 |
| | | STRA = for | | | | | | | |
| 8 | 103371_at | Cluster Incl AF100956: Mus musculus major histocompatibility locus class II region; Fas-binding protein Daxx (DAXX) gene, | 652.3 | P | 838.1 | P | 766.9 | P | 851.7 |
| | | partial cds; Bing1 (BING1), tapasin (tapasin), RalGDS-like factor (RLF), KE2 (KE2), BING4 (BING4), beta1, 3-galactosyl | | | | | | | |
| | | transferase (be/STRA = rev | | | | | | | |
| 8 | 103630_at | Cluster Incl AI844089 UI-M-AL1-ahi-h-12-0-UI s1 Mus musculus cDNA, 3 end/clone = UI-M-AL1-ahi-h-12-0-UI/ | 352.5 | P | 485.7 | P | 317.4 | P | 450.6 |
| | | clone_end = 3/gb = AI844089/gi = 5487995/ug = Mm.20290/len = 241/STRA = for | | | | | | | |
| 8 | 103821_at | Cluster Incl AJ223087: Mus musculus mRNA for Cdc6-related protein/cds = (103,1872)/gb = AJ223087/gi = 4007644/ | 232.2 | P | 224.8 | P | 160.4 | P | 201.2 |
| | | ug = Mm 20912/len = 2589/STRA = for | | | | | | | |
| 8 | 103910_at | Cluster Incl AJ249987: Mus musculus TAFII30 gene for mTAFII30 protein, exons 1-5/cds = (25,681)/gb = AJ249987/ | 848.7 | P | 1183.5 | P | 593.8 | P | 742.7 |
| | | gi = 6119622/ug = Mm.21419/len = 759/STRA = for | | | | | | | |
| 8 | 104057_at | Cluster Incl AW012807 uo03a03 y1 Mus musculus cDNA, 5 end/clone = IMAGE-2582284/clone_end = 5/gb = AW012807/ | 326.2 | P | 316.6 | P | 261.9 | P | 305.8 |
| | | gi = 5861585/ug = Mm 21535/len = 544/STRA = for | | | | | | | |
| 8 | 104279_at | Cluster Incl AW125116 UI-M-BH2.1-apx-h-12-0-UI s1 Mus musculus cDNA, 3 end/clone = UI-M-BH2.1-apx-h-12-0-UI/ | 1461.5 | P | 1478.6 | P | 1369.6 | P | 1621.6 |
| | | clone_end = 3/gb = AW125116/gi = 6100646/ug = Mm 22230/len = 442/STRA = for | | | | | | | |
| 8 | 104371_at | Cluster Incl AF078752 Mus musculus diacylglycerol acyltransferase (Dgat) mRNA, complete cds/cds = (95,1591)/ | 368.7 | P | 530.7 | P | 369.4 | P | 488 |
| | | gb = AF078752/gi = 3859933/ug = Mm 22633/len = 1650/STRA = for | | | | | | | |
| 8 | 104560_at | Cluster Incl AW125634: UI-M-BH2.2-aqj-h-11-0-UI s1 Mus musculus cDNA, 3 end/clone = UI-M-BH2.2-aqj-h-11-0-UI/ | 304.1 | P | 247.8 | P | 257.4 | P | 365.1 |
| | | clone_end = 3/gb = AW125634/gi = 6101164/ug = Mm 23827/len = 447/STRA = for | | | | | | | |
| 8 | 104573_at | Cluster Incl AA921069 vy83h09.r1 Mus musculus cDNA, 5 end/clone = IMAGE-1312865/clone_end = 5/gb = AA921069/ | 482.8 | P | 527.2 | P | 395.5 | P | 598.4 |
| | | gi = 3067848/ug = Mm 23925/len = 263/STRA = for | | | | | | | |
| 8 | 104633_at | Cluster Incl AW123921. UI-M-BH2.3-aqc-a-07-0-UI s1 Mus musculus cDNA, 3 end/clone = UI-M-BH2 3-aqc-a-07-0-UI/ | 520.3 | P | 630.6 | P | 454.9 | P | 502.6 |
| | | clone_end = 3/gb = AW123921/gi = 6099451/ug = Mm 24342/len = 236/STRA = for | | | | | | | |
| 8 | 94327_at | Cluster Incl AW230209 up28c10 y1 Mus musculus cDNA, 5 end/clone = IMAGE-2655666/clone_end = 5/gb = AW230209/ | 594.2 | P | 557.1 | P | 548.3 | P | 524.1 |
| | | gi = 6559505/ug = Mm 24875/len = 259/STRA = for | | | | | | | |
| 8 | 94340_at | Cluster Incl AW124224 UI-M-BH2.1-aph-d-08-0-UI s1 Mus musculus cDNA, 3 end/clone = UI-M-BH2.1-aph-d-08-0-UI/ | 363.4 | P | 377.4 | P | 338.3 | P | 275.3 |
| | | clone_end = 3/gb = AW124224/gi = 6099754/ug = Mm 25042/len = 383/STRA = for | | | | | | | |
| 8 | 95631_at | Cluster Incl AF088911: Mus musculus protein phosphatase X (Ppx) mRNA, complete cds/cds = (139,1062)/gb = AF088911/ | 1574.7 | P | 1792.5 | P | 1114.9 | P | 1871.8 |
| | | gi = 4028534/ug = Mm 28082/len = 1276/STRA = rev | | | | | | | |
| 8 | 96122_at | Cluster Incl AW049373 UI-M-BH1-ane-b-11-0-UI s1 Mus musculus cDNA, 3 end/clone = UI-M-BH1-ane-b-11-0-UI/ | 393.5 | P | 365.7 | P | 460.8 | P | 386 |
| | | clone_end = 3/gb = AW049373/gi = 5909902/ug = Mm.28108/len = 580/STRA = for | | | | | | | |
| 8 | 96237_at | Cluster Incl AI118905 uc15f04 r1 Mus musculus cDNA, 5 end/clone = IMAGE-1398079/clone_end = 5/gb = AI118905/ | 561.8 | P | 526.7 | P | 212.2 | P | 665.8 |
| | | gi = 3519229/ug = Mm 28800/len = 311/STRA = for | | | | | | | |
| 8 | 96834_at | Cluster Incl AI843586 UI-M-AO1-aeh-f-08-0-UI s1 Mus musculus cDNA, 3 end/clone = UI-M-AO1-aeh-f-08-0-UI/ | 1374.1 | P | 1308.4 | P | 1137.2 | P | 1116.5 |
| | | clone_end = 3/gb = AI843586/gi = 5477764/ug = Mm 29426/len = 398/STRA = for | | | | | | | |
| 8 | 97342_at | Cluster Incl AW060867 UI-M-BH1-amx-f-08-0-UI s1 Mus musculus cDNA, 3 end/clone = UI-M-BH1-amx-f-08-0-UI/ | 1384.1 | P | 1399.2 | P | 1221 | P | 1300.8 |
| | | clone_end = 3/gb = AW060867/gi = 6008618/ug = Mm 29599/len = 418/STRA = for | | | | | | | |
| 8 | 98446_s_at | Cluster Incl U06834 Mus musculus Balb/c eph-related receptor protein tyrosine kinase mRNA, complete cds/ | 698.8 | P | 646.6 | P | 597 | P | 833.5 |
| | | cds = (102,3065)/gb = U06834/gi = 459172/ug = Mm 34533/len = 3733/STRA = rev | | | | | | | |
| 8 | 98495_at | Cluster Incl AI846906.UI-M-AK1-aey-b-11-0-UI.s1 Mus musculus cDNA, 3 end/clone = UI-M-AK1-aey-b-11-0-UI/ | 454.5 | P | 517.5 | P | 531.9 | P | 422.7 |
| | | clone_end = 3/gb = AI846908/gi = 5490812/ug = Mm 3561/len = 336/STRA = for | | | | | | | |
| 8 | 100007_at | Cluster Incl AI837573 UI-M-AL0-abs-g-06-0-UI.s1 Mus musculus cDNA, 3 end/clone = UI-M-AL0-abs-g-06-0-UI/ | 1113.9 | P | 1020.1 | P | 940.4 | P | 1020.8 |
| | | clone_end = 3/gb-AI837573/gi = 5471786/ug = Mm 4532/len = 293/STRA = for | | | | | | | |
| 8 | 100959_at | Cluster Incl X99921.M musculus mRNA for S100 calcium-binding protein A13/cds = (60,356)/gb = X99921/gi = 1694840/ | 1438.5 | P | 1738.1 | P | 1358.7 | P | 1631.4 |
| | | ug = Mm.6523/len = 440/STRA = for | | | | | | | |
| 8 | 100964_at | Cluster Incl AF035208 Mus musculus putative v-SNARE Vti1b mRNA, complete cds/cds = (11,709)/gb = AF035208/ | 870.9 | P | 862.5 | P | 747.7 | P | 796 |
| | | gi = 3213228/ug = Mm 6577/len = 909/STRA = for | | | | | | | |
| 8 | 100970_at | Cluster Incl X65687: Thymoma viral proto-oncogene/cds = (283,1725)/gb = X65687/gi = 287806/ug = Mm 6845/len = 2626/ | 844.8 | P | 918.6 | P | 974.8 | P | 1061.1 |
| | | STRA = for | | | | | | | |
| 8 | 100990_g_at | Cluster Incl AJ001373 Mus musculus bodenin gene/cds = (173,775)/gb = AJ001373/gi = 2647955/ug = Mm 6818/len = 954/ | 451.8 | P | 484.9 | P | 414 | P | 485.1 |
| | | STRA = for | | | | | | | |
| 8 | 101449_at | Cluster Incl AW120841 UI-M-BH2.3-aoa-e-02-0-UI s1 Mus musculus cDNA, 3 end/clone = UI-M-BH2.3-aoa-e-02-0-UI/ | 135.1 | P | 172.6 | P | 163.5 | P | 191 |
| | | clone_end = 3/gb = AW120841/gi = 6096174/ug = Mm 794/len = 283/STRA = for | | | | | | | |
| 8 | 101461_f_at | Cluster Incl U06944 Praja1/cds = (328,1602)/gb = U06944/gi = 2828789/ug = Mm 8211/len = 1978/STRA = for | 1720 | P | 1881.1 | P | 1520.7 | P | 1445.5 |
| 8 | 101905_at | Cluster Incl AF037454: lichy/cds = (149,2713)/gb = AF037454/gi = 2827197/ug = Mm 8975/len = 5122/STRA = for | 1557.2 | P | 1453.6 | P | 1417 | P | 1547.8 |
| 8 | 101931_at | Cluster Incl AF042731: Mus musculus phosphatidylserine synthase-1 mRNA, complete cds/cds = (93,1514)/gb = AF042731/ | 856.4 | P | 847.9 | P | 983.1 | P | 1038.3 |
| | | gi = 2811227/ug = Mm 9440/len = 2455/STRA = for | | | | | | | |
| 8 | 102235_at | Cluster Incl X13945 Lung carcinoma myc related oncogene 1/cds = (0,1106)/gb = X13945/gi = 53287/ug = Mm.1055/ | 382.3 | P | 425.7 | P | 430.5 | P | 352.1 |
| | | len = 3076/STRA = for | | | | | | | |
| 8 | 94105_at | Cluster Incl L78075: Cell division cycle 42/cds = (0,575)/gb = L78075/gi = 1321598/ug = Mm 1022/len = 576/STRA = for | 1494 | P | 1707 | P | 1490.2 | P | 1517.8 |
| 8 | 100345_f_at | Cluster Incl W65964: me10a08 r1 Mus musculus cDNA, 5 and/clone = IMAGE-387062/clone_end = 5/gb = W65964/ | 1905.5 | P | 2030.2 | P | 1492 | P | 1554.3 |
| | | gi = 1373784/ug = Mm.88247/len = 404/STRA = rev | | | | | | | |
| 8 | 97060_at | Cluster Incl AW215489 up04f11 x1 Mus musculus cDNA, 3 end/clone = IMAGE-2651085/clone_end = 3/gb = AW215489/ | 396.7 | P | 447.7 | P | 347 | P | 375.2 |
| | | gi = 6526165/ug = Mm.89290/len = 522/STRA = rev | | | | | | | |
| 8 | 100733_at | Cluster Incl X70303 Proteasome (prosome, macropain) subunit, alpha type 2/cds = (77,781)/gb = X70303/gi = 897783/ | 2276.3 | P | 2129.1 | P | 1648.8 | P | 2136.9 |
| | | ug = Mm 3428/len = 904/STRA = for | | | | | | | |
| 8 | 93252_at | Cluster Incl X81816 M musculus BAP31 mRNA/cds = (2,739)/gb = X81816/gi = 1487985/ug = Mm 17/len = 1132/STRA = for | 741.7 | P | 819 | P | 853.5 | A | 1004.9 |
| 8 | 93741_at | Cluster Incl AA693066: vr61b07.s1 Mus musculus cDNA, 5 end/clone = IMAGE-1125109/clone_end = 5/gb = AA693066/ | 247 | P | 276.5 | P | 229.6 | P | 235 |
| | | gi = 2694004/ug = Mm.21062/len = 379/STRA = for | | | | | | | |
| 8 | 94237_at | Cluster Incl AI846708.UI-M-AN1-afi-g-08-0-UI s1 Mus musculus cDNA, 3 end/clone = UI-M-AN1-afi-g-08-o-UI | 554.7 | P | 564.5 | P | 348.7 | P | 478.8 |
| | | clone_end = 3/gb = AI846708/gi = 5490614/ug = Mm 22333/len = 342/STRA = for | | | | | | | |
| 8 | 98921_at | Cluster Incl AW125612: UI-M-BH2.2-aqj-f-12-0-UI s1 Mus musculus cDNA, 3 end/clone = UI-M-BH2.2-aqj-f-12-0-UI/ | 425.8 | P | 433.6 | P | 473 | P | 511.6 |
| | | clone_end = 3/gb = AW125812/gi = 6101142/ug = Mm 28626/len = 449/STRA = for | | | | | | | |
| 8 | 98936_at | Cluster Incl AI837395: UI-M-AP0-abm-d-04-0-UI s1 Mus musculus cDNA, 3 end/clone = UI-M-AP0-abm-d-04-0-UI/ | 320.3 | P | 257.5 | P | 441.7 | P | 347 |
| | | clone_end = 3/gb = AI837395/gi = 5471608/ug = Mm 28688/len = 328/STRA = for | | | | | | | |
| 8 | 95052_at | Cluster Incl AI839150 UI-M-AO0-ach-e-07-0-UI s1 Mus musculus cDNA, 3 end/clone = UI-M-AO0-ach-e-07-0-UI/ | 347 | P | 402.9 | P | 270.4 | P | 386.6 |
| | | clone_end = 3/gb = AI839150/gi = 5473363/ug = Mm 29140/len = 462/STRA = for | | | | | | | |
| 8 | 99671_at | Cluster Incl X04673 Adipsin/cds = (19,798)/gb = X04673/gi = 49883/ug = Mm4407/len = 867/STRA = for | 3457.6 | P | 2627.2 | P | 1691.7 | P | 3483.3 |
| 8 | 102016_at | Cluster Incl M61737: M. musculus adipocyte-specific mRNA, partial cds/cds = UNKNOWN/gb = M61737/gi = 201107/ | 269.5 | P | 384.2 | P | 228.2 | P | 260.9 |
| | | ug = Mm 10026/len = 1725/STRA = for | | | | | | | |
| 8 | 102400_at | Cluster Incl AW125118 UI-M-BH2 1-apz-a-03-0-UI s1 Mus musculus cDNA, 3 end/clone = UI-M-BH2.1-apz-a-03-0-UI/ | 235.3 | P | 287.1 | P | 179.6 | P | 286.6 |
| | | clone_end = 3/gb = AW125118/gi = 6100648/ug = Mm 12441/len = 270/STRA = for | | | | | | | |
| 8 | 103398_at | Cluster Incl AW123232: UI-M-BH2 1-apd-g-08-0-UI s1 Mus musculus cDNA, 3 end/clone = UI-M-BH2 1-apd-g-08-0-UI/ | 204.4 | P | 183.3 | P | 232.2 | P | 279.5 |
| | | clone_end = 3/gb = AW123232/gi = 6098727/ug = Mm 18714/len = 469/STRA = rev | | | | | | | |
| 8 | 103646_at | Cluster Incl X85983 Carnitine acetyltransferase/cds = (125,2008)/gb = X85983/gi = 758631/ug = Mm 20396/len = 2596/ | 269.8 | P | 407.9 | P | 224.6 | A | 322.3 |
| | | STRA = for | | | | | | | |
| 8 | 104381_at | Cluster Incl AF085745 Mus musculus nuclear orphan receptor LXR-alpha mRNA, complete cds/cds = (84,1421)/ | 66.2 | P | 106.6 | P | 88.3 | P | 101.2 |
| | | gb = AF085745/gi = 4322470/ug = Mm 22690/len = 1665/STRA = rev | | | | | | | |
| 8 | 100900_at | Cluster Incl AW209053: un99c08 x1 Mus musculus cDNA, 3 end/clone = IMAGE-2582030/clone_end = 3/gb = AW209053/ | 504.3 | P | 414.6 | P | 469.9 | P | 413.6 |
| | | gi = 6514993/ug = Mm 57203/len = 446/STRA = for | | | | | | | |
| 8 | 101393_at | Cluster Incl AJ001633: Annexin III/cds = (200,1171)/gb = AJ001633/gi = 2437839/ug = Mm 7214/len = 1602/STRA = for | 108.1 | P | 104 | P | 101 | P | 186.2 |
| 8 | 104444_at | Cluster Incl AA689927 v163c12 r1 Mus musculus cDNA, 5 end/clone = IMAGE-1167766/clone_end = 5/gb = AA689927/ | 237.8 | P | 277.1 | P | 237.4 | P | 274.7 |
| | | gi = 2690863/ug = Mm 29321/len = 163/STRA = for | | | | | | | |
| 8 | 96598_at | Cluster Incl AA122714 mr02b11 r1 Mus musculus cDNA, 5 end/clone = IMAGE-5296253/clone_end = 5/gb = AA122714/ | 38.9 | P | 88 | P | 90 | P | 53.7 |
| | | gi = 1681720/ug = Mm 309/len = 506/STRA = for | | | | | | | |
| 8 | 93080_at | Cluster Incl AF069954 G protein gamma 3 linked gene/cds = (305,1537)/gb = AF069954/gi = 3885499/ug = Mm 15985/ | 624.5 | P | 821 | P | 1052.1 | P | 744.5 |
| | | len = 1654/STRA = for | | | | | | | |
| 8 | 93342_at | Cluster Incl AI852665: UI-M-BH0-aji-b-02-0-UI s1 Mus musculus cDNA, 3 end/clone = UI-M-BH0-aji-b-02-0-UI/clone_end = 3/ | 206.3 | P | 202.5 | P | 270.1 | P | 175.5 |
| | | gb = AI852665/gi = 5496571/ug = Mm 18739/len = 503/STRA = for | | | | | | | |
| 8 | 94080_at | Cluster Incl AI835718: UI-M-AI0-aan-c-10-0-UI s1 Mus musculus cDNA, 3 end/clone = UI-M-AI0-aan-c-10-0-UI/ | 177.8 | P | 199.6 | P | 119.8 | P | 185.5 |
| | | clone_end = 3/gb = AI835718/gi = 5469931/ug = Mm 22149/len = 348/STRA = for | | | | | | | |
| 8 | 94313_at | Cluster Incl X96767: M musculus mRNA for U1 anRNP-specific protein C/cds = (31,510)/gb = X96767/gi = 1360016/ | 478.1 | P | 495.7 | P | 398.6 | P | 485.9 |
| | | ug = Mm 2265/len = 738/STRA = for | | | | | | | |
| 8 | 95427_at | Cluster Incl AI843650: UI-M-AO1-aen-h-06-0-UI s1 Mus musculus cDNA, 3 end/clone = UI-M-AO1-aen-h-06-0-UI/ | 552.9 | P | 760.4 | P | 442.5 | P | 392.9 |
| | | clone_end = 3/gb = AI843650/gi = 5477863/ug = Mm 24455/len = 449/STRA = for | | | | | | | |
| 8 | 98538_at | Cluster Incl X81632 M musculus mRNA for e1 protein/cds = UNKNOWN/gb = X81632/gi = 563507/ug = Mm 35854/len = 3769/ | 324.1 | P | 484.5 | P | 263 | P | 513.8 |
| | | STRA = for | | | | | | | |
| 8 | 101526_at | Cluster Incl X14759 Homeo box, msh-like 1/cds = (141,1112)/gb = X14759/gi = 51431/ug = Mm 870/len = 1810/STRA = for | 801.4 | P | 699.4 | P | 389 | P | 711.9 |
| 8 | 103853_at | Cluster Incl AI845664 UI-M-AQ1-ady-c-07-0-UI s1 Mus musculus cDNA, 3 end/clone = UI-M-AQ1-ady-c-07-0-UI/ | 114 | P | 115.8 | P | 51.1 | M | 124.7 |
| | | clone_end = 3/gb = AI845664/gi = 5489570/ug = Mm 21070/len = 184/STRA = for | | | | | | | |
| 8 | 104120_at | Cluster Incl AW049205 UI-M-BH1-anb-a-02-0-UI s1 Mus musculus cDNA, 3 end/clone = UI-M-BH1-anb-a-02-0-UI/ | 860.2 | P | 897.1 | P | 910 | P | 879.5 |
| | | clone_end = 3/gb = AW049205/gi = 5909734/ug = Mm 21978/len = 412/STRA = for | | | | | | | |
| 8 | 104287_at | Cluster Incl AI853807 UI-M-BH0-ajg-h-06-0-UI s1 Mus musculus cDNA, 3 end/clone = UI-M-BH0-ajg-h-06-0-UI/ | 357.6 | P | 460.6 | P | 568.5 | P | 487 |
| | | clone_end = 3/gb = AI853807/gi = 5497713/ug = Mm.22271/len = 445/STRA = for | | | | | | | |
| 8 | 97934_at | Cluster Incl U73820 Mus musculus polypeptide GalNac transferase-T1 (ppGaNTase-T1) mRNA, complete cds/ | 193.9 | A | 173.3 | P | 162 | P | 188.9 |
| | | cds = (0,1679)/gb = U73820/gi = 2149048/ug = Mm.30249/len = 1680/STRA = for | | | | | | | |
| 8 | 103506_f_at | Cluster Incl AW228162 up14b05 x1 Mus musculus cDNA, 3 end/clone = IMAGE-2654289/clone_end = 3/gb = AW228162/ | 52.3 | P | 141.9 | P | 91.1 | P | 134 |
| | | gi = 6557458/ug = Mm 2247/len = 414/STRA = for | | | | | | | |
| 8 | 92805_s_at | Cluster Incl Y12577 ADP-ribosylation-like 4/cds = (243,845)/gb = Y12577/gi = 3288541/ug = Mm 12723/len = 1168/STRA = for | 161.4 | P | 272 | P | 242 | P | −54.3 |
| 8 | 95453_f_at | Cluster Incl AF087687 Mus musculus calcium-binding protein S100A1 mRNA, complete cds/cds = (110,394)/gb = AF087687/ | 294.5 | P | 253.6 | P | 183.5 | P | 279.6 |
| | | gi = 3746891/ug = Mm.24662/len = 506/STRA = for | | | | | | | |
| 8 | 95480_at | Cluster Incl AI847163 UI-M-AO1-aep-d-12-0-UI s1 Mus musculus cDNA, 3 end/clone = UI-M-AO1-aep-d-12-0-UI/ | 31.1 | P | 173.3 | A | 96.5 | P | −5.3 |
| | | clone_end = 3/gb = AI847163/gi = 5491069/ug = Mm 24855/len = 148/STRA = for | | | | | | | |
| 8 | 96110_at | Cluster Incl U31966: Carbonyl reductase/cds = (128,961)/gb = U31966/gi = 1049107/ug = Mm.26940/len = 1053/STRA = for | 362.6 | P | 375.4 | P | 367.1 | P | 306.8 |
| 8 | 96604_at | Cluster Incl AW125827 UI-M-BH2 2-aqk-b-10-0-UI s1 Mus musculus cDNA, 3 end/clone = UI-M-BH2 2-aqk-b-10-0-UI/ | 165.9 | P | 204.7 | P | 189.9 | P | 206.1 |
| | | clone_end = 3/gb = AW125827/gi = 6101357/ug = Mm 27039/len = 461/STRA = for | | | | | | | |
| 8 | 98920_g_at | Cluster Incl AA867773: vx16d06.r1 Mus musculus cDNA, 5 end/clone = IMAGE-1284619/clone_end = 5/gb = AA867773/ | 232 | P | 208.7 | P | 260.7 | P | 292.8 |
| | | gi = 2963218/ug = Mm.28626/len = 385/STRA = for | | | | | | | |
| 8 | 98588_at | Cluster Incl Z11774 Fumarylacetoacetate hydrolase/cds = (31,1290)/gb = Z11774/gi = 50972/ug = Mm 3798/len = 1398/ | 256.9 | P | 298.9 | P | 374.6 | P | 360.6 |
| | | STRA = rev | | | | | | | |
| 8 | 102232_at | Cluster Incl AI850740 UI-M-BG1-ail-g-10-0-UI s1 Mus musculus cDNA, 3 end/clone = UI-M-BG1-ail-g-10-0-UI/ | 153.6 | P | 177.6 | P | 147.2 | A | 181.3 |
| | | clone_end = 3/gb = AI850740/gi = 5494646/ug = Mm 1040/len = 404/STRA = for | | | | | | | |
| 8 | 103735_at | Cluster Incl M89800 Wingless-related MMTV integration site 6/cds = (0,1094)/gb = M89800/gi = 202407/ug = Mm 2438/ | 54.1 | P | 138.9 | A | 44.6 | P | 111.6 |
| | | len = 1435/STRA = for | | | | | | | |
| 8 | 104695_at | Cluster Incl AI639864 vx72d06 x1 Mus musculus cDNA, 3 end/clone = IMAGE-1280747/clone_end = 3/gb = AI639864/ | 244.4 | P | 355.3 | P | 230.5 | P | 301.1 |
| | | gi = 4702973/ug = Mm.33622/len = 347/STRA = for | | | | | | | |
| 8 | 94514_s_at | Cluster Incl AI853439 UI-M-BH0-ajy-d-04-0-UI s1 Mus musculus cDNA, 3 end/clone = UI-M-BH0-ajy-d-04-0-UI/ | 338.8 | P | 357.5 | P | 46.1 | A | 124.8 |
| | | clone_end = 3/gb = AI853439/gi = 5497345/ug = Mm 28981/len = 465/STRA = for | | | | | | | |
| 8 | 99146_at | Cluster Incl AW124355 UI-M-BH2 1-apq-h-02-0-UI s1 Mus musculus cDNA, 3 end/clone = UI-M-BH2 1-apq-h-02-0-UI/ | 185.3 | P | 94 | P | 130.7 | P | 195.7 |
| | | clone_end = 3/gb = AW124355/gi = 6099885/ug = Mm 41462/len = 237/STRA = for | | | | | | | |
| 8 | 99600_at | Cluster Incl AI838337: UI-M-AO0-aby-h-03-0-UI.s1 Mus musculus cDNA, 3 end/clone = UI-M-AO0-aby-h-03-0-UI/ | 239 | A | 316.7 | P | 357.5 | P | 232.2 |
| | | clone_end = 3/gb = AI838337/gi = 5472550/ug = Mm.42855/len = 414/STRA = for | | | | | | | |
| 8 | 104132_at | Cluster Incl AW047276: UI-M-BH1-amh-c-02-0-UI.s1 Mus musculus cDNA, 3 end/clone = UI-M-BH1-amh-c-02-0-UI/ | 174.7 | P | 135.8 | A | 91.8 | P | 182.2 |
| | | clone_end = 3/gb = AW047276/gi = 5907805/ug = Mm 22072/len = 602/STRA = for | | | | | | | |
| 8 | 104318_at | Cluster Incl Z97018: Mus musculus mRNA for PEX5 protein/cds = (0,1919)/gb = Z97018/gi = 2222731/ug = Mm.22418/ | 82.9 | A | 118.9 | P | 33.9 | M | 108.2 |
| | | len = 1986/STRA = for | | | | | | | |
| 8 | 94956_at | Cluster Incl AF021031 DiGeorge syndrome chromosome region 6/cds = (0,584)/gb = AF021031/gi = 2582382/ug = Mm.27155/ | 150 | P | 72 | P | 56.9 | P | 29.4 |
| | | len = 996/STRA = for | | | | | | | |
| 8 | 99988_at | Cluster Incl AW122115 UI-M-BH2 2-aor-b-02-0-UI s1 Mus musculus cDNA, 3 end/clone = UI-M-BH2 2-aor-b-02-0-UI/ | 110.1 | P | 174.8 | P | 27.9 | P | 47.9 |
| | | clone_end = 3/gb = AW122115/gi = 6097532/ug = Mm.4471/len = 403/STRA = for | | | | | | | |
| 8 | 101728_at | Cluster Incl S46665 C5a anaphylatoxin receptor = peptidergic G-protein-coupled receptor [mice, BALB/C, Genomic, 1114 nt | 687.6 | A | 983.4 | P | 742.2 | P | 1004.8 |
| | | 2 segments]/cds = (0,1055)/gb = S46665/gi = 1680365/ug = Mm.57044/len = 1056/STRA = for | | | | | | | |
| 8 | 94020_at | Cluster Incl Y17808 Mus musculus mRNA for A6 related protein/cds = (17,1066)/gb = Y17808/gi = 4468631/ug = Mm 21852/ | 150.1 | A | 207.6 | P | 157.1 | A | 179.7 |
| | | len = 1435/STRA = for | | | | | | | |
| 8 | 96020_at | Cluster Incl M22531: Mouse complement C1q B chain mRNA, complete cds/cds = (156,917)/gb = M22531/gi = 192266/ | 218 | P | 324.1 | P | 305.5 | P | 266.6 |
| | | ug = Mm.2570/len = 1059/STRA = for | | | | | | | |
| 8 | 96071_at | Cluster Incl AI835709 UI-M-AI0-aan-b-12-0-UI.s1 Mus musculus cDNA, 3 end/clone = UI-M-AI0-aan-b-12-0-UI/ | 1204.8 | A | 1721 | A | 1497 | A | 1517 |
| | | clone_end = 3/gb = AI835709/gi = 5469922/ug = Mm 265/len = 395/STRA = for | | | | | | | |
| 8 | 97252_at | Cluster Incl AW120926 UI-M-BH2.3-aob-f-08-0-UI s1 Mus musculus cDNA, 3 end/clone = UI-M-BH2 3-aob-f-08-0-UI/ | 143.4 | P | 154.8 | P | −20.6 | P | 141.4 |
| | | clone_end = 3/gb = AW120926/gi = 6096259/ug = Mm 27871/len = 171/STRA = for | | | | | | | |
| 8 | 95738_at | Cluster Incl AW124889 UI-M-BH2.1-apu-c-11-0-UI s1 Mus musculus cDNA, 3 end/clone = UI-M-BH2 1-apu-c-11-0-UI/ | 136 | P | 116.1 | M | 99.2 | P | 126.3 |
| | | clone_end = 3/gb = AW124889/gi = 6100419/ug = Mm 29751/len = 386/STRA = for | | | | | | | |
| 8 | 98606_s_at | Cluster Incl X69656 M musculus (clone S5) WRS mRNA for tryptophan-tRNA ligase/cds = (0,1427)/gb = X69656/gi = 55435/ | 210.1 | A | 192.8 | P | 191.2 | P | 229.4 |
| | | ug = Mm 38433/len = 1428/STRA = for | | | | | | | |
| 8 | 101027_s_at | Cluster Incl AF069051 Mus musculus pituitary tumor transfroming gene protein (PTTG) mRNA, complete cds/ | 409.7 | P | 544.4 | P | 503.5 | P | 345.9 |
| | | cds = (303,893)/gb = AF069051/gi = 3702843/ug = Mm 6856/len = 945/STRA = for | | | | | | | |
| 8 | 103643_at | Cluster Incl AI844784 UI-M-AL1-ahq-g-12-0-UI s1 Mus musculus cDNA, 3 end/clone = UI-M-AL1-ahq-g-12-0-UI/ | 219.9 | P | 263.9 | A | 153.8 | P | 212.9 |
| | | clone_end = 3/gb = AI844784/gi = 5488690/ug = Mm 20387/len = 264/STRA = rev | | | | | | | |
| 8 | 104354_at | Cluster Incl X06368 Colony stimulating factor 1 receptor/cds = (75,3005)/gb = X06368/gi = 50980/ug = Mm.22574/len = 3665/ | 400.4 | P | 493.2 | P | 460.1 | P | 298.9 |
| | | STRA = for | | | | | | | |
| 8 | 104719_at | Cluster Incl AI182203 uc68a04 r1 Mus musculus cDNA, 5 end/clone = IMAGE-1430766/clone_end = 5/gb = AI182203/ | 1.5 | A | 167.8 | P | 34.5 | P | 179 |
| | | gi = 3732841/ug = Mm 24510/len = 517/STRA = for | | | | | | | |
| 8 | 92182_at | Cluster Incl AI836877 UI-M-AP0-abg-c-08-0-UI s2 Mus musculus cDNA, 3 end/clone = UI-M-AP0-abg-c-08-0-UI/ | 174 | P | 98.4 | M | 83.1 | M | 166.8 |
| | | clone_end = 3/gb = AI836877/gi = 5471090/ug = Mm 33851/len = 395/STRA = for | | | | | | | |
| 8 | 93856_at | Cluster Incl M55512 Wilms tumor homolog/cds = UNKNOWN/gb = M55512/gi = 202413/ug = Mm 87561/len = 3089/ | 211.5 | P | 184.4 | P | 188.6 | P | 120.9 |
| | | STRA = for | | | | | | | |
| 8 | 94512_f_at | Cluster Incl AI843210 UI-M-AK1-aet-f-05-0-UI s1 Mus musculus cDNA, 3 end/clone = UI-M-AK1-aet-f-05-0-UI/ | 228.1 | P | 202.9 | A | 102.7 | A | 157.2 |
| | | clone_end = 3/gb = AI843210/gi = 5477423/ug = Mm 28981/len = 418/STRA = for | | | | | | | |
| 8 | 94515_at | Cluster Incl AW208628 uo62a08 x1 Mus musculus cDNA, 3 end/clone = IMAGE-2647094/clone_end = 3/gb = AW208628/ | 50 | P | 74.9 | P | 60 | A | 98.9 |
| | | gi = 6514568/ug = Mm.28986/len = 601/STRA = for | | | | | | | |
| 8 | 94561_at | Clustar Incl AI836140 UI-M-AQ0-aai-b-09-0-UI s2 Mus musculus cDNA, 3 end/clone = UI-M-AQ0-aai-b-09-0-UI/ | 538.6 | A | 600 | P | 416.4 | A | 442 |
| | | clone_end = 3/gb = AI836140/gi = 5470353/ug = Mm 29107/len = 498/STRA = for | | | | | | | |
| 8 | 97531_at | Cluster Incl X95280 Mus musculus mRNA for GOS2-like protein/cds = (132,443)/gb = X95280/gi = 1213012/ug = Mm 3283/ | 261.5 | P | 303.5 | P | 279 | A | 304.1 |
| | | len = 752/STRA = for | | | | | | | |
| 8 | 101062_at | Cluster Incl AF054670: Heme oxygenase (decycling) 2/cds = (98,1045)/gb = AF054670/gi = 2984775/ug = Mm 710/len = 1192/ | 57.4 | A | 56.3 | P | 3.5 | A | 100.3 |
| | | STRA = for | | | | | | | |
| 8 | 102366_at | Cluster Incl AA718169 yu54h05 r1 Mus musculus cDNA, 5 end/clone = IMAGE-1195257/clone_end = 5/gb = AA718169/ | 782.5 | P | 820.6 | P | 407.1 | P | 821.3 |
| | | gi = 2730443/ug = Mm 1181/len = 290/STRA = for | | | | | | | |
| 8 | 101842_g_at | Cluster Incl AW049716 UI-M-BH1-ani-e-11-0-UI s1 Mus musculus cDNA, 3 end/clone = UI-M-BH1-ani-e-11-0-UI/ | 238.9 | P | 230.1 | P | 83.1 | M | 241.5 |
| | | clone_end = 3/gb = AW049716/gi = 5910245/ug = Mm 8534/len = 524/STRA = for | | | | | | | |
| 8 | 93613_at | Cluster Incl AB008192 Mus musculus mRNA for mSmad3, complete cds/cds = (26,1303)/gb = AB008192/gi = 4630836/ | 62.2 | P | 29.6 | A | 45.7 | A | 45.1 |
| | | ug = Mm 7320/len = 1428/STRA = for | | | | | | | |
| 9 | 92611_at | Cluster Incl U18773 Mus musculus GPI-anchored protein p137 mRNA, partial cds/cds = (0,550)/gb = U18773/gi = 862634/ | 1436.1 | P | 1542.3 | P | 917.5 | P | 1077.3 |
| | | ug = Mm.1098/len = 2707/STRA = for | | | | | | | |
| 9 | 92623_at | Cluster Incl AI847171: UI-M-AO1-aep-e-08-0-UI s1 Mus musculus cDNA, 3 end/clone = UI-M-AO1-aep-e-08-0-UI/ | 1909.5 | P | 1638.2 | P | 1645.1 | P | 1463 |
| | | clone_end = 3/gb = AI847171/gi = 5491077/ug = Mm 11204/len = 208/STRA = for | | | | | | | |
| 9 | 92639_at | Cluster Incl U80932 Senne/threonine kinase 6/cds = (48,1235)/gb = U80932/gi = 1763646/ug = Mm.11738/len = 1643/ | 152.7 | P | 220.2 | P | 119 | P | 157.2 |
| | | STRA = for | | | | | | | |
| 9 | 92644_s_at | Cluster Incl M12848 Mouse myb proto-oncogene mRNA encoding 71 kd myb protein, complete cds/cds = (161,2071)/ | 139 | P | 156 | P | 135 | P | 115 |
| | | gb = M12848/gi = 199934/ug = Mm 1202/len = 3310/STRA = for | | | | | | | |
| 9 | 92647_at | Cluster Incl U35141: Mus musculus retinoblastoma-binding protein (mRbAp48) mRNA, complete cds/cds = (107,1492)/ | 923.7 | P | 937.1 | P | 785.9 | P | 712.2 |
| | | gb = U35141/gi = 1016274/ug = Mm.12145/len = 1961/STRA = for | | | | | | | |
| 9 | 92790_at | Cluster Incl D55720 Karyopherin (importin) alpha 2/cds = (177,1766)/gb = D55720/gi = 893392/ug = Mm 12508/len = 2048/ | 726.1 | P | 618.5 | P | 412.7 | P | 432.6 |
| | | STRA = for | | | | | | | |
| 9 | 92809_r_at | Cluster Incl X17069 Mouse mRNA for protein with homology to transition protein 2 (TP2)/cds = (123,1358)/gb = X17069/ | 1831.5 | P | 1405.3 | P | 1122.1 | P | 1241 |
| | | gi = 54875/ug = Mm 12758/len = 2026/STRA = for | | | | | | | |
| 9 | 92826_at | Cluster Incl Y17852 Mus musculus mRNA for ganglioside-induced differentiation associated protein 3/cds = (0,385)/ | 405.4 | P | 296.2 | P | 310.7 | P | 286.2 |
| | | gb = Y17852/gi = 3378457/ug = Mm 12962/len = 651/STRA = for | | | | | | | |
| 9 | 92829_at | Cluster Incl U09659 Mus musculus chaperonin 10 mRNA, complete cds/cds = (78,386)/gb = U09659/gi = 495205/ | 1806.8 | P | 1436.9 | P | 1184.3 | P | 1545.3 |
| | | ug = Mm.12970/len = 462/STRA = for | | | | | | | |
| 9 | 93013_at | Cluster Incl AF077861 Inhibitor of DNA binding 2/cds = (0,404)/gb = AF077861/gi = 3420740/ug = Mm.1466/len = 405/ | 781.8 | P | 482.8 | P | 546.6 | P | 614 |
| | | STRA = for | | | | | | | |
| 9 | 93041_at | Cluster Incl D26089: Mini chromosome maintenance deficient 4 homolog (S. cerevisiae)/cds = (126,2714)/gb = D26089/ | 486.7 | P | 445.5 | P | 188.6 | P | 292.2 |
| | | gi = 940405/ug = Mm.1500/len = 3221/STRA = for | | | | | | | |
| 9 | 93051_at | Cluster Incl Z37107: M. musculus mRNA for epoxide hydrolase/cds = (17,1681)/gb = Z37107/gi = 563509/ug = Mm.15295/ | 151.5 | P | 201.4 | P | 176.7 | P | 132 |
| | | len = 1990/STRA = for | | | | | | | |
| 9 | 93069_at | Cluster Incl U62483 Mus musculus ubiquitin conjugating enzyme (ubc4) mRNA, complete cds/cds = (386,829)/gb = U62483/ | 217.7 | P | 204.8 | P | 230.7 | P | 104.6 |
| | | gi = 1480741/ug = Mm 1570/len = 2410/STRA = for | | | | | | | |
| 9 | 93095_at | Cluster Incl U00431: Mus musculus HMG-1 mRNA, complete cds/cds = (145,792)/gb = U00431/gi = 437101/ug = Mm 16421/ | 668.4 | P | 495.3 | P | 452 | P | 249.6 |
| | | len = 1308/STRA = for | | | | | | | |
| 9 | 93117_at | Cluster Incl AF073993: Mus musculus heterogenous nuclear ribonucleoprotein A2/B1 (hnRNP A2/B1) mRNA, complete cds/ | 3304.3 | P | 2330.4 | P | 1529.5 | P | 1698.3 |
| | | cds = (96,1121)/gb = AF073993/gi = 3329497/ug = Mm 16767/len = 1677/STRA = for | | | | | | | |
| 9 | 93250_r_at | Cluster Incl X67668 High mobility group protein 2/cds = (135,752)/gb = X67668/gi = 51338/ug = Mm 1693/len = 847/ | 1190.5 | P | 1283.1 | P | 760.7 | P | 767.5 |
| | | STRA = for | | | | | | | |
| 9 | 93512_f_at | Cluster Incl AW121801 UI-M-BH2 3-anx-b-02-0-UI s2 Mus musculus cDNA, 3 end/clone = UI-M-BH2 3-anx-b-02-0-UI/ | 568.4 | P | 579.5 | P | 508.8 | P | 455.8 |
| | | clone_end = 3/gb = AW121801/gi = 6097161/ug = Mm 19352/len = 406/STRA = for | | | | | | | |
| 9 | 93519_s_at | Cluster Incl AI847056 UI-M-AP1-agn-a-10-0-UI s1 Mus musculus cDNA, 3 end/clone = UI-M-AP1-agn-a-10-0-UI/ | 1308.6 | P | 1358.2 | P | 1112.8 | P | 1145.1 |
| | | clone_end = 3/gb = AI847056/gi = 5490962/ug = Mm 196/len = 185/STRA = for | | | | | | | |
| 9 | 93541_at | Cluster Incl Z68618 Transgelin/cds = (12,617)/gb = Z68618/gi = 1160197/ug = Mm 2006/len = 1016/STRA = for | 841.7 | P | 741.8 | P | 515 | P | 778.8 |
| 9 | 93562_at | Cluster Incl AI854366 UI-M-BG1-aic-e-10-0-UI s1 Mus musculus cDNA, 3 end/clone = UI-M-BG1-aic-e-10-0-UI/ | 1348.8 | P | 1380.6 | P | 1273.7 | P | 918.4 |
| | | clone_end = 3/gb = AI854366/gi = 5498272/ug = Mm 2033/len = 410/STRA = for | | | | | | | |
| 9 | 93772_i_at | Cluster Incl AI835041 UI-M-AI1-afn-d-02-0-UI s1 Mus musculus cDNA, 3 end/clone = UI-M-AI1-afn-d-02-0-UI/clone_end = 3/ | 1044.1 | P | 736.5 | P | 480.4 | P | 516.3 |
| | | gb = AI835041/gi = 5469254/ug = Mm 21203/len = 409/STRA = for | | | | | | | |
| 9 | 93786_i_at | Cluster Incl AI837302 UI-M-AK0-ade-h-11-0-UI s1 Mus musculus cDNA, 3 end/clone = UI-M-AK0-ade-h-11-0-UI | 677.8 | P | 689.1 | P | 510 | P | 594 |
| | | clone_end = 3/gb = AI837302/gi = 5471515/ug = Mm 21356/len = 392/STRA = for | | | | | | | |
| 9 | 93787_f_at | Cluster Incl AI837302: UI-M-AK0-ade-h-11-0-UI s1 Mus musculus cDNA, 3 end/clone = UI-M-AK0-ade-h-11-0-UI/ | 1930.9 | P | 1840.5 | P | 1391 | P | 1391.8 |
| | | clone_end = 3/gb = AI837302/gi = 5471515/ug-Mm 21356/len = 392/STRA = for | | | | | | | |
| 9 | 93805_at | Cluster Incl AW121164 UI-M-BH2.3-aoh-g-12-0-UI s1 Mus musculus cDNA, 3 end/clone = UI-M-BH2 3-aoh-g-12-0-UI/ | 694.4 | P | 741.6 | P | 471.5 | P | 542 |
| | | clone_end = 3/gb = AW121164/gi = 6096497/ug = Mm 21444/len = 423/STRA = for | | | | | | | |
| 9 | 93820_at | Cluster Incl AF037371 Mus musculus cytochrome c oxidase subunit VIIa-L precursor (Cox7al) mRNA, nuclear gene | 1937.9 | P | 2078.4 | P | 1812.6 | P | 1727.5 |
| | | encoding mitochondrial protein, complete cds/cds = (70,321)/gb = AF037371/gi = 2708817/ug = Mm.2151/len = 461/ | | | | | | | |
| | | STRA = for | | | | | | | |
| 9 | 93997_at | Cluster Incl AI853475 UI-M-BH0-ajy-h-08-0-UI s1 Mus musculus cDNA, 3 end/clone = UI-M-BH0-ajy-h-08-0-UI/ | 434.9 | P | 418.7 | P | 371.9 | P | 398 |
| | | clone_end = 3/gb = AI853475/gi = 5497381/ug-Mm 21761/len = 472/STRA = for | | | | | | | |
| 9 | 93999_at | Cluster Incl AW060597 UI-M-BH1-anq-d-09-0-UI s1 Mus musculus cDNA, 3 end/clone = UI-M-BH1-anq-d-09-0-UI/ | 1659.8 | P | 1569.9 | P | 1241.7 | P | 1357.8 |
| | | clone_end = 3/gb = AW060597/gi = 6008348/ug = Mm 21764/len = 224/STRA = for | | | | | | | |
| 9 | 94030_at | Cluster Incl AI853431: UI-M-BH0-ajy-c-06-0-UI s1 Mus musculus cDNA, 3 end/clone = UI-M-BH0-ajy-c-06-0-UI/ | 233.2 | P | 262.3 | P | 190.5 | P | 224.2 |
| | | clone_end = 3/gb = AI853431/gi = 5497337/ug = Mm 21901/len = 250/STRA = for | | | | | | | |
| 9 | 94073_at | Cluster Incl AI836182 UI-M-AQ0-aaj-h-11-0-UI s2 Mus musculus cDNA, 3 end/clone = UI-M-AQ0-aaj-h-11-0-UI/ | 769.5 | P | 699 | P | 547.4 | P | 604.6 |
| | | clone_end = 3/gb = AI836182/gi = 5470395/ug = Mm.22117/len = 542/STRA = for | | | | | | | |
| 9 | 94228_at | Cluster Incl AW123788 UI-M-BH2.1-apm-d-07-0-UI s1 Mus musculus cDNA, 3 end/clone = UI-M-BH2 1-apm-d-07-0-UI/ | 519.6 | P | 457.8 | P | 162.8 | P | 265.5 |
| | | clone_end = 3/gb = AW123788/gi = 6099318/ug-Mm 22269/len = 174/STRA = rev | | | | | | | |
| 9 | 94267_i_at | Cluster Incl AW124386 UI-M-BH2 1-ape-b-11-0-UI s1 Mus musculus cDNA, 3 end/clone = UI-M-BH2 1-ape-d-11-0-UI/ | 1681.6 | P | 1154.1 | P | 1333.9 | P | 1003.8 |
| | | clone_end = 3/gb = AW124386/gi = 6099916/ug = Mm.22470/len = 121/STRA = for | | | | | | | |
| 9 | 94268_f_at | Cluster Incl AW124386 UI-M-BH2 1-ape-b-11-0-UI s1 Mus musculus cDNA, 3 end/clone = UI-M-BH2 1-ape-b-11-0-UI/ | 1968.1 | P | 2027.3 | P | 2071.3 | P | 1704.5 |
| | | clone_end = 3/gb = AW124386/gi = 6099916/ug = Mm 22470/len = 121/STRA = for | | | | | | | |
| 9 | 94294_at | Cluster Incl X66032 M musculus mRNA for cyclin B2/cds = (101,1297)/gb = X66032/gi = 50612/ug = Mm 22592/len = 1528/ | 1256.1 | P | 1162.6 | P | 1067.2 | P | 823.9 |
| | | STRA = for | | | | | | | |
| 9 | 94806_at | Cluster Incl AW125336 UI-M-BH2 1-apy-g-01-0-UI s1 Mus musculus cDNA, 3 end/clone = UI-M-BH2.1-apy-g-01-0-UI/ | 527.9 | P | 916.5 | P | 808.5 | P | 691.5 |
| | | clone_end = 3/gb = AW125336/gi = 6100866/ug = Mm 22676/len = 253/STRA = for | | | | | | | |
| 9 | 94885_at | Cluster Incl AI840062 UI-M-AL0-abt-b-05-0-UI s1 Mus musculus cDNA, 3 end/clone = UI-M-AL0-abt-b-05-0-UI/ | 240.5 | P | 94.9 | P | 156.7 | P | 216.1 |
| | | clone_end = 3/gb = AI840062/gi = 5474275/ug = Mm 23889/len = 499/STRA = for | | | | | | | |
| 9 | 95423_at | Cluster Incl Y00884 Mouse mRNA (3 4J) from concanavalin A-stimulated LB3 T-cells/cds = (22,1770)/gb = Y00884/ | 525.5 | P | 485.9 | P | 420.1 | P | 498.6 |
| | | gi = 49727/ug = Mm 2442/len = 1957/STRA = for | | | | | | | |
| 9 | 95451_at | Cluster Incl AA855382: vw72a04.r1 Mus musculus cDNA, 5 end/clone = IMAGE-1260462/clone_end = 5/gb = AA855382/ | 498.1 | P | 448.1 | P | 302.6 | P | 316.3 |
| | | gi = 2942920/ug = Mm 24641/len = 314/STRA = for | | | | | | | |
| 9 | 95479_at | Cluster Incl X95591 M. musculus mRNA for C1D protein/cds = (77,502)/gb = X95591/gi = 1185124/ug = Mm 2485/len = 1050/ | 267 | P | 224.8 | P | 184.2 | P | 196.7 |
| | | STRA = for | | | | | | | |
| 9 | 96054_f_at | Cluster Incl Y17345 Mus musculus mRNA for protein-tyrosine-phosphatase IF1/cds = (0,476)/gb = Y17345/gi = 3294228/ | 564.1 | P | 442.6 | P | 294.4 | P | 384.3 |
| | | ug = Mm 26166/len = 477/STRA = for | | | | | | | |
| 9 | 96658_at | Cluster Incl AI841906: UI-M-AO0-acd-e-10-0-UI s1 Mus musculus cDNA, 3 end/clone = UI-M-AO0-acd-e-10-0-UI/ | 693.4 | P | 589.5 | P | 540.8 | P | 432.3 |
| | | clone_end = 3/gb = AI841906/gi = 5476119/ug = Mm 27344/len = 417/STRA = for | | | | | | | |
| 9 | 96676_at | Cluster Incl AW121875 UI-M-BH2.3-aoi-d-05-0-UI s1 Mus musculus cDNA, 3 end/clone = UI-M-BH2 3-aoi-d-05-0-UI/ | 608.6 | P | 482.1 | P | 354.3 | P | 524.6 |
| | | clone_end = 3/gb = AW121875/gi = 6097249/ug = Mm.27406/len = 438/STRA = for | | | | | | | |
| 9 | 96693_at | Cluster Incl AI849453 UI-M-AJ1-ahc-h-03-0-UI s1 Mus musculus cDNA, 3 end/clone = UI-M-AJ1-ahc-h-03-0-UI/ | 793.4 | P | 664.1 | P | 697.4 | P | 580.3 |
| | | clone_end = 3/gb = AI849453/gi = 5493359/ug = Mm.27526/len = 291/STRA = for | | | | | | | |
| 9 | 96707_at | Cluster Incl D10630: Zinc finger protein 38/cds = (105,1772)/gb = D10630/ug = Mm 2760/len = 2103/STRA = for | 299.3 | P | 343.5 | P | 232.9 | P | 244.7 |
| 9 | 96711_at | Cluster Incl AI849191 UI-M-AJ1-agy-g-09-0-UI s1 Mus musculus cDNA, 3 end/clone = UI-M-AJ1-agy-g-09-0-UI/ | 339.1 | P | 369.9 | P | 332.8 | P | 299.4 |
| | | clone_end = 3/gb = AI849191/gi = 5493097/ug = Mm.27635/len = 469/STRA = for | | | | | | | |
| 9 | 97200_f_at | Cluster Incl X65704 M musculus mRNA for small nuclear ribonucleoprotein E/cds = (0,245)/gb = X65704/gi = 312004/ | 3067.9 | P | 3291.2 | P | 2444.1 | P | 2374.3 |
| | | ug = Mm.27669/len = 387/STRA = for | | | | | | | |
| 9 | 97201_s_at | Cluster Incl AA823381: vp39a01.r1 Mus musculus cDNA, 5 end/clone = IMAGE-1079016/clone_end = 5/gb = AA823381/ | 1162.5 | P | 1319.2 | P | 766.3 | P | 926.3 |
| | | gi = 2893249/ug = Mm 27677/len = 339/STRA = for | | | | | | | |
| 9 | 97254_at | Cluster Incl AA690061: vr7Bd03.s1 Mus musculus cDNA, 5 end/clone = IMAGE-1134821/clone_end = 5/gb = AA690061/ | 514 | P | 514.9 | P | 428.7 | P | 441.2 |
| | | gi = 2690997/ug = Mm.27875/len = 331/STRA = for | | | | | | | |
| 9 | 97296_at | Cluster Incl AW124918: UI-M-BH2.1-apu-f-07-0-UI.s1 Mus musculus cDNA, 3 end/clone = UI-M-BH2.1-apu-f-07-0-UI/ | 219.8 | P | 215.6 | P | 137.4 | P | 177.1 |
| | | clone _end = 3/gb = AW124918/gi = 6100448/ug = Mm.28039/len = 187/STRA = for | | | | | | | |
| 9 | 97301_at | Cluster Incl AW124187: UI-M-BH2.1-aph-a-06-0-UI.s1 Mus musculus cDNA, 3 end/clone = UI-M-BH2.1-aph-a-06-0-UI/ | 1383.5 | P | 1430.7 | P | 1179 | P | 1164.4 |
| | | clone_end = 3/gb = AW124187/gb = 6099717/ug = Mm.28045/len = 382/STRA = for | | | | | | | |
| 9 | 97307_f_at | Cluster Incl AI842808: UI-M-AP1-agi-f-09-0-UI.s1 Mus musculus cDNA, 3 end/clone = UI-M-AP1-agi-f-09-0-UI/clone_end = 3/ | 2971.3 | P | 3424.9 | P | 2949.5 | P | 2631.7 |
| | | gb = AI842808/gi = 5477021/ug = Mm.28058/len = 337/STRA = for | | | | | | | |
| 9 | 97315_at | Cluster Incl AW121730: UI-M-BH2.3-aoe-b-02-0-UI.s1 Mus musculus cDNA, 3 end/clone = UI-M-BH2.3-aoe-b-02-0-UI/ | 490.7 | P | 490.3 | P | 420.8 | P | 400.7 |
| | | clone _end = 3/gb = AW121730/gi = 6097063/ug = Mm.28097/len = 372/STRA = for | | | | | | | |
| 9 | 97317_at | Cluster Incl AW122933: UI-M-BH2.1-apa-h-09-0-UI.s1 Mus musculus cDNA, 3 end/clone = UI-M-BH2.1-apa-h-09-0-UI/ | 563.6 | P | 578.4 | P | 531.5 | P | 438.3 |
| | | clone_end = 3/gb = AW122933/gi = 6098463/ug = Mm.28107/len = 399/STRA = for | | | | | | | |
| 9 | 97846_at | Cluster Incl AI848968: UI-M-AJ1-ahd-c-01-0-UI.s1 Mus musculus cDNA, 3 end/clone = UI-M-AJ1-ahd-c-01-0-UI/ | 649.6 | P | 628.8 | P | 398.7 | P | 335.9 |
| | | clone_end = 3/gb = AI848968/gb = 5492874/ug = Mm.28270/len = 391/STRA = for | | | | | | | |
| 9 | 97900_at | Cluster Incl AI845714: UI-M-AQ1-ady-h-04-0-UI.s1 Mus musculus cDNA, 3 end/clone = UI-M-AQ1-ady-h-04-0-UI/ | 583.2 | P | 443.4 | P | 422.2 | P | 428 |
| | | clone_end = 3/gb = AI845714/gi = 5489620/ug = Mm.28438/len = 384/STRA = for | | | | | | | |
| 9 | 98914_at | Cluster Incl AI853634: UI-M-BH0-ajp-h-08-0-UI.s1 Mus musculus cDNA, 3 end/clone = UI-M-BH0-ajp-h-08-0-UI/ | 622.3 | P | 628.7 | P | 575.6 | P | 467.5 |
| | | clone_end = 3/gb = AI853634/gi = 5497540/ug = Mm.28606/len = 374/STRA = for | | | | | | | |
| 9 | 98915_at | Cluster Incl AI849082: UI-M-AH1-agw-e-11-0-UI.s1 Mus musculus cDNA, 3 end/clone = UI-M-AH1-agw-e-11-0-UI/ | 607.8 | P | 537.4 | P | 560.1 | P | 595.2 |
| | | clone_end = 3/gb = AI849082/gi = 5492988/ug = Mm.28614/len = 494/STRA = for | | | | | | | |
| 9 | 94489_at | Cluster Incl U84411: Mus musculus protein tyrosine phosphatase (PRL-1) mRNA, complete cds/cds = (713,1234)/ | 2306.8 | P | 1939.7 | P | 2378.6 | P | 1890.2 |
| | | gb = U84411/gi = 1814023/ug = Mm.28909/len = 2849/STRA = for | | | | | | | |
| 9 | 94508_at | Cluster Incl AI853772: UI-M-BH0-ajg-e-02-0-UI.s1 Mus musculus cDNA, 3 end/clone = UI-M-BH0-ajg-e-02-0-UI/ | 241.4 | P | 166.8 | P | 167.4 | P | 187.8 |
| | | clone_end = 3/gb = AI853772/gi = 5497678/ug = Mm.28963/len = 394/STRA = for | | | | | | | |
| 9 | 95045_at | Cluster Incl AI844469: UI-M-AJ1-ahe-d-03-0-UI.s1 Mus musculus cDNA, 3 end/clone = UI-M-AJ1-ahe-d-03-0-UI/ | 583.2 | P | 454.1 | P | 339.3 | P | 343 |
| | | clone_end = 3/gb = AI844489/gi = 5488375/ug = Mm.29122/len = 406/STRA = for | | | | | | | |
| 9 | 95063_at | Cluster Incl AI606257: vo03b06 x1 Mus musculus cDNA, 3 end/clone = IMAGE-1040339/clone_end = 3/gb = AI606257/ | 349.4 | P | 312 | P | 151.7 | P | 254.3 |
| | | gi = 4615424/ug = Mm.29164/len = 366/STRA = for | | | | | | | |
| 9 | 95069_at | Cluster Incl AA940430: vz48h05 r1 Mus musculus cDNA, 5 end/clone = IMAGE-1329753/clone_end = 5/gb = AA940430/ | 621.3 | P | 856.3 | P | 583.8 | P | 490 |
| | | gi = 3099974/ug = Mm.29187/len = 468/STRA = for | | | | | | | |
| 9 | 95105_at | Cluster Incl AI847697: UI-M-AP1-agm-d-08-0-UI.s1 Mus musculus cDNA, 3 end/clone = UI-M-AP1-agm-d-08-0-UI/ | 1055.8 | P | 1329.6 | P | 1193.1 | P | 1059.9 |
| | | clone_end = 3/gb = AI847697/gi = 5491603/ug = Mm.29353/len = 441/STRA = for | | | | | | | |
| 9 | 95109_at | Cluster Incl AW121447: UI-M-BH2.2-aon-d-07-0-UI.s1 Mus musculus cDNA, 3 end/clone = UI-M-BH2.2-aon-d-07-0-UI/ | 490.4 | P | 465.4 | P | 237.1 | P | 397 |
| | | clone_end = 3/gb = AW121447/gi = 6096780/ug = Mm.29363/len = 278/STRA = for | | | | | | | |
| 9 | 95119_at | Cluster Incl AA866888: vx90e09 r1 Mus musculus cDNA, 5 end/clone = IMAGE-1282504/clone_end = 5/gb = AA866888/ | 283.1 | P | 233.8 | P | 165.6 | P | 121.6 |
| | | gi = 2962333/ug = Mm 29382/len = 420/STRA = for | | | | | | | |
| 9 | 95673_s_at | Cluster Incl AW124113: UI-M-BH2.1-apr-b-09-0-UI.s1 Mus musculus cDNA, 3 end/clone = UI-M-BH2.1-apr-b-09-0-UI/ | 2431.6 | P | 2353.7 | P | 2263.5 | P | 2101.6 |
| | | clone_end = 3/gb = AW124113/gi = 6099643/ug = Mm.29588/len = 265/STRA = for | | | | | | | |
| 9 | 95699_f_at | Cluster Incl AI848094: UI-M-AP1-agk-d-05-0-UI.s1 Mus musculus cDNA, 3 end/clone = UI-M-AP1-agk-d-05-0-UI/ | 1241.9 | P | 1162.8 | P | 1136.7 | P | 975.4 |
| | | clone_end = 3/gb = AI848094/gi = 5492000/ug = Mm.29685/len = 399/STRA = for | | | | | | | |
| 9 | 96865_at | Cluster Incl M60474: Mynstoylated alanine rich protein kinase C substrate/cds = (380,1309)/gb = M60474/gi = 199026/ | 1404.6 | P | 1591.3 | P | 1031 | P | 812.2 |
| | | ug = Mm.30059/len = 2597/STRA = for | | | | | | | |
| 9 | 96891_at | Cluster Incl AI842771: UI-M-AP1-agi-c-05-0-UI.s1 Mus musculus cDNA, 3 end/clone = UI-M-AP1-agi-c-05-0-UI/ | 2606.4 | P | 2341.9 | P | 2130.4 | P | 1849.1 |
| | | clone_end = 3/gb = AI842771/gi = 5476984/ug = Mm.30093/len = 354/STRA = for | | | | | | | |
| 9 | 96899_at | Cluster Incl AW123802: UI-M-BH2.1-apm-e-09-0-UI.s1 Mus musculus cDNA, 3 end/clone = UI-M-BH2.1-apm-e-09-0-UI/ | 1191.1 | P | 1419 | P | 1173.8 | P | 1163.5 |
| | | clone_end = 3/gb = AW123802/gi = 6099332/ug = Mm.30113/len = 371/STRA = for | | | | | | | |
| 9 | 96947_at | Cluster Incl AW046273: UI-M-BH1-ala-f-05-0-UI.s1 Mus musculus cDNA, 3 end/clone = UI-M-BH1-ala-f-05-0-UI/ | 1252.3 | P | 1527.7 | P | 1052.3 | P | 1206.4 |
| | | clone_end = 3/gb = AW046273/gi = 5906802/ug = Mm.30200/len = 374/STRA = for | | | | | | | |
| 9 | 96951_at | Cluster Incl AI839795: UI-M-AP0-abf-a-02-0-UI.s2 Mus musculus cDNA, 3 end/clone = UI-M-AP0-abf-a-02-0-UI/ | 832.9 | P | 739.5 | P | 669.3 | P | 699.1 |
| | | clone_end = 3/gb = AI839795/gi = 5474048/ug = Mm.30206/len = 475/STRA = for | | | | | | | |
| 9 | 97445_at | Cluster Incl AW122022: UI-M-BH2.2-aoo-a-09-0-UI.s1 Mus musculus cDNA, 3 end/clone = UI-M-BH2.2-aoo-a-09-0-UI/ | 386.1 | P | 343.1 | P | 293.7 | P | 264.2 |
| | | clone_end = 3/gb = AW122022/gi = 6097428/ug = Mm.30242/len = 507/STRA = for | | | | | | | |
| 9 | 97468_at | Cluster Incl AB025409: Mus musculus mRNA for aid1334p, complete cds/cds = (78,317)/gb = AB025409/gi = 5931566/ | 681.3 | P | 671.6 | P | 507.5 | P | 554.7 |
| | | ug = Mm.3049/len = 757/STRA = for | | | | | | | |
| 9 | 97474_f_at | Cluster Incl D90225: Pleiotrophin/cds = (214,720)/gb = D90225/gi = 220553/ug = Mm.3063/len = 1514/STRA = for | 193 | P | 173.3 | P | 152.4 | P | 72.3 |
| 9 | 97478_at | Cluster Incl AW046965: UI-M-BH1-alp-c-09-0-UI.s1 Mus musculus cDNA, 3 end/clone = UI-M-BH1-alp-c-09-0-UI/ | 311.4 | P | 385.7 | P | 305.8 | P | 279.1 |
| | | clone_end = 3/gb = Aw046965/gi = 5907494/ug = Mm.30718/len = 368/STRA = for | | | | | | | |
| 9 | 97527_at | Cluster Incl AA681998: vr45a06 s1 Mus musculus cDNA, 5 end/clone = IMAGE-1123570/clone_end = 5/gb = AA681998/ | 685.8 | P | 531.3 | P | 416 | P | 422.7 |
| | | gi = 2664138/ug = Mm.326/len = 463/STRA = for | | | | | | | |
| 9 | 97543_at | Cluster Incl D49382: Mus musculus Nedd5 mRNA for septin, complete cds/cds = (53,1138)/gb = D49382/gi = 4519167/ | 1171.4 | P | 1037.5 | P | 747.2 | P | 689.7 |
| | | ug = Mm.336/len = 3170/STRA = for | | | | | | | |
| 9 | 97556_at | Cluster Incl AI843178: UI-M-AK1-aet-c-07-0-UI.s1 Mus musculus cDNA, 3 end/clone = UI-M-AK1-aet-c-07-0-UI/ | 671 | P | 690.6 | P | 514.7 | P | 408.2 |
| | | clone_end = 3/gb = AI843178/gi = 5477391/ug = Mm.33875/len = 452/STRA = for | | | | | | | |
| 9 | 98038_at | Cluster Incl AF022465: Mus musculus high mobility group protein homolog HMG4 (Hmg4) mRNA, complete cds/ | 1283.4 | P | 1346.8 | P | 1109.6 | P | 887 |
| | | cds = (27,829)/gb = AF022465/gi = 2738988/ug = Mm.340/len = 1502/STRA = for | | | | | | | |
| 9 | 98072_f_at | Cluster Incl X77731: M. musculus mRNA for Deoxycytidine kinase/cds = (12,794)/gb = X77731/gi = 456676/ug = Mm.3446 | 95.4 | P | 82.2 | P | 41.7 | P | 74.4 |
| | | len = 2812/STRA = for | | | | | | | |
| 9 | 98075_at | Cluster Incl AW121683: UI-M-BH2.3-aoc-e-07-0-UI.s1 Mus musculus cDNA, 3 end/clone = UI-M-BH2.3-aoc-e-07-0-UI/ | 391.1 | P | 352.6 | P | 217 | P | 232.6 |
| | | clone_end = 3/gb = AW121683/gi = 6097916/ug = Mm.34526/len = 543/STRA = for | | | | | | | |
| 9 | 98128_at | Cluster Incl U77128: Mus musculus mitochondrial ATP synthase coupling factor 6 mRNA, nuclear gene encoding | 1206.7 | P | 1224.1 | P | 898.6 | P | 973.2 |
| | | mitochondrial protein, complete cds/cds = (7,333)/gb = U77128/gi = 1679742/ug = Mm.353/len = 425/STRA = for | | | | | | | |
| 9 | 98550_at | Cluster Incl AI854006: UI-M-BH0-alv-h-10-0-UI.s1 Mus musculus cDNA, 3 end/clone = UI-M-BH0-alv-h-10-0-UI/ | 1528 | P | 2059.4 | P | 995.6 | P | 1508 |
| | | clone_end = 3/gb = AI854006/gi = 5497912/ug = Mm.36677/len = 407/STRA = for | | | | | | | |
| 9 | 98573_r_at | Cluster Incl X58045: RAN binding protein 1/cds = (137,748)/gb = X56045/gi = 443997/ug = Mm 3752/len = 1044/STRA = for | 1052.9 | P | 1051.4 | P | 774.1 | P | 824.5 |
| 9 | 98610_at | Cluster Incl AW047926 UI-M- BH1-aln-c-12-0-UI s1 Mus musculus cDNA, 3 end/clone = UI-M-BH1-aln-c-12-0-UI/ | 203.8 | P | 208.1 | P | 187.5 | P | 202.4 |
| | | clone_end = 3/gb = AW047926/gi = 5908455/ug = Mm 38460/len = 359/STRA = for | | | | | | | |
| 9 | 98613_at | Cluster Incl AI849876 UI-M-AL1-aho-h-03-0-UI.s1 Mus musculus cDNA, 3 end/clone = UI-M-AL1-aho-h-03-0-UI/ | 284.9 | P | 516.9 | P | 151.9 | P | 142.7 |
| | | clone_end = 3/gb = AI849876/gi = 5493775/ug = Mm 38521/len = 416/STRA = for | | | | | | | |
| 9 | 98615_at | Cluster Incl AW049570 UI-M-BH1-anf-h-02-0-UI s1 Mus musculus cDNA, 3 end/clone = UI-M-BH1-anf-h-02-0-UI/ | 807.7 | P | 706.5 | P | 552.8 | P | 694.2 |
| | | clone_end = 3/gb = AW049570/gi = 5910099/ug = Mm 38524/len = 232/STRA = for | | | | | | | |
| 9 | 99594_at | Cluster Incl AI844598 UI-M-AJ1-ahh-c-08-0-UI s2 Mus musculus cDNA, 3 end/clone = UI-M-AJ1-ahh-c-08-0-UI/ | 1169.4 | P | 1296.2 | P | 1054.8 | P | 1139 |
| | | clone_end = 3/gb = AI844598/gi = 5488509/ug = Mm 42826/len = 329/STRA = for | | | | | | | |
| 9 | 99632_at | Cluster Incl U83902 Mus musculus mitotic checkpoint component Mad2 mRNA, complete cds/cds = (48,665)/gb = U83902/ | 412.4 | P | 287 | P | 219.5 | P | 218.1 |
| | | gi = 4099130/ug = Mm 43444/len = 707/STRA = for | | | | | | | |
| 9 | 99645_at | Cluster Incl AW048484 UI-M-BH1-alj-d-10-0-UI.s1 Mus musculus cDNA, 3 end/clone = UI-M-BH1-adj-d-10-o-UI/ | 645.6 | P | 698.7 | P | 517.6 | P | 533.1 |
| | | clone_end = 3/gb = AW048484/gi = 5909018/ug = Mm 43640/len = 458/STRA = for | | | | | | | |
| 9 | 100113_s_at | Cluster Incl D50367: Mus musculus mRNA for KAP3B, complete cds/cds = (193,2511)/gb = D50367/gi = 1526420/ | 268 | P | 235.7 | P | 212 | P | 186 |
| | | ug = Mm 4651/len = 3908/STRA = for | | | | | | | |
| 9 | 100116_at | Cluster Incl AI22538 uc62107 r1 Mus musculus cDNA, 5 end/clone = IMAGE-1430245/clone_end = 5/gb = AI122538/ | 1016.6 | P | 935.8 | P | 735.5 | P | 537.4 |
| | | gi = 3522662/ug = Mm.46673/len = 505/STRA = for | | | | | | | |
| 9 | 100522_s_at | Cluster Incl U92454 Mus musculus WW domain binding protein 5 mRNA, partial cds/cds = (0,185)/gb = U92454/ | 2299.3 | P | 2447.7 | P | 1841.4 | P | 1732 |
| | | gi = 1914850/ug = Mm 512/len = 647/STRA = for | | | | | | | |
| 9 | 100523_r_at | Cluster Incl U92454 Mus musculus WW domain binding protein 5 mRNA, partial cds/cds = (0,185)/gb = U92454/ | 508.6 | P | 511.3 | P | 400.9 | P | 319.8 |
| | | gi = 1914850/ug = Mm.512/len = 647/STRA = for | | | | | | | |
| 9 | 100588_at | Cluster Incl U60329: Protease (prosome, macropain) 28 subunit, beta/cds = (1,720)/gb = U60329/gi = 1698571/ug = Mm 626/ | 1150.2 | P | 1003.3 | P | 665.2 | P | 948.3 |
| | | len = 723/STRA = for | | | | | | | |
| 9 | 101001_at | Cluster Incl AI647612: uk41h01 x1 Mus musculus cDNA, 3 end/clone = IMAGE-1971601/clone_end = 3/gb = AI647612/ | 1231.8 | P | 1411.3 | P | 1062.5 | P | 827.9 |
| | | gi = 4726290/ug = Mm.6766/len = 524/STRA = for | | | | | | | |
| 9 | 101002_at | Cluster Incl AF032128 Mus musculus antizyms inhibitor mRNA, complete cds/cds = (16,1362)/gb = AF032128/gi = 2645700/ | 599.8 | P | 476 | P | 347.6 | P | 367.9 |
| | | ug = Mm.6775/len = 1599/STRA = for | | | | | | | |
| 9 | 101004_f_at | Cluster Incl X91658: Splicing factor, arginine/serine-rich 3 (SRp20)/cds = (116,490)/gb = X91658/gi = 2125862/ug = Mm 6787/ | 1147.2 | P | 1198.2 | P | 843 | P | 1080.6 |
| | | len = 1895/STRA = for | | | | | | | |
| 9 | 101056_at | Cluster Incl X60672 M musculus mRNA for radixin/cds = (165,19166)/gb = X60672/gi = 1033049/ug = Mm.7051/len = 4238/ | 1158.9 | P | 1121.2 | P | 925.9 | P | 779.5 |
| | | STRA = for | | | | | | | |
| 9 | 101060_at | Cluster Incl M73329: Mouse phospholipase C-alpha (PCL-alpha) mRNA, complete cds/cds = (79,1593)/gb = M73329/ | 1444.5 | P | 1272.1 | P | 1115.8 | P | 1065.4 |
| | | gi = 200396/ug = Mm.709/len = 1900/STRA = for | | | | | | | |
| 9 | 101064_at | Cluster Incl AF044334: Mus musculus plelotropic regulator 1 (PLRG1) mRNA, complete cds/cds = (0,1541)/gb = AF044334/ | 730.7 | P | 664.6 | P | 525 | P | 577.4 |
| | | gi = 2832297/ug = Mm.7138/len = 1542/STRA = for | | | | | | | |
| 9 | 101065_at | Cluster Incl X57800: Proliferating cell nuclear antigen/cds = (144,929)/gb = X57800/gi = 53601/ug = Mm.7141/len = 1261/ | 1880.7 | P | 1621.5 | P | 831.6 | P | 1210.6 |
| | | STRA = for | | | | | | | |
| 9 | 101067_at | Cluster Incl AW124711: UI-M-BH2 1-aqa-a-12-0-UI.s1 Mus musculus cDNA, 3 end/clone = UI-M-BH2.1-aqa-a-12-0-UI/ | 915.7 | P | 851 | P | 831.9 | P | 516.7 |
| | | clone_end = 3/gb = AW124711/gi = 6100241/ug = Mm 719/len = 467/STRA = for | | | | | | | |
| 9 | 101091_at | Cluster Incl AF033353 Ubiquitin-like 1/cds = (118,423)/gb = AF033353/gi = 2645736/ug = Mm.7353/len = 1187/STRA = for | 1204.8 | P | 1087.9 | P | 945.8 | P | 948.3 |
| 9 | 101506_at | Cluster Incl AW227345 up 11a09 y1 Mus musculus cDNA, 5 end/clone = IMAGE-2651704/clone_end = 5/gb = AW227345/ | 404.8 | P | 460 | P | 203.1 | P | 400 |
| | | gi = 6556710/ug = Mm 821/len = 447/STRA = for | | | | | | | |
| 9 | 101509_at | Cluster Incl U96760 Mus musculus von Hippel-Lindau binding protein homolog (vbp-1) mRNA, complete cds/ | 839.6 | P | 685 | P | 479.2 | P | 486.3 |
| | | cds = (101,583)/gb = U96760/gi = 2738245/ug = Mm 8294/len = 1531/STRA = for | | | | | | | |
| 9 | 101580_at | Cluster Incl AI851220 UI-M-BH0-ejx-g-09-0-UI s1 Mus musculus cDNA, 3 end/clone = UI-M-BH0-ajx-g-09-0-UI/ | 1869.8 | P | 1839.6 | P | 1050.6 | P | 1663.6 |
| | | clone_end = 3/gb = AI851220/gi = 5495126/ug = Mm.90007/len = 344/STRA = for | | | | | | | |
| 9 | 101958_f_at | Cluster Incl X72310: Transcription factor Dp 1/cds = (54,1286)/gb = X72310/gi = 395280/ug = Mm 925/len = 1700/STRA = for | 655.3 | P | 672.3 | P | 390.7 | P | 555.6 |
| 9 | 101959_r_at | Cluster Incl X72310 Transcription factor Dp 1/cds = (54,1286)/gb = X72310/gi = 395280/ug = Mm 925/len = 1700/STRA = for | 942 | P | 986.3 | P | 546.1 | P | 782.9 |
| 9 | 102001_at | Cluster Incl M14223 Mouse ribonucleotide reductase M2 subunit mRNA, complete cds/cds = (62,1234)/gb = M14223/ | 468.3 | P | 483.8 | P | 382.1 | P | 374.2 |
| 9 | 102409_at | Cluster Incl AW046963 UI-M-BH1-alp-c-c7-0-UI s1 Mus musculus cDNA, 3 end/clone = UI-M-BH1-alp-c-07-0-UI/ | 414.3 | P | 526.9 | P | 412.2 | P | 414.7 |
| | | clone_end = 3/gb = AW046963/gi = 5907492/ug = Mm 12539/len = 307/STRA = for | | | | | | | |
| 9 | 102776_at | Cluster Incl AI55095 ud59b08 r1 Mus musculus cDNA, 5 end/clone = IMAGE-1450163/clone_end = 5/gb = AI55095/ | 218.5 | P | 211.9 | P | 56.9 | P | 179.3 |
| | | gi = 3683564/ug = Mm 13437/len = 120/STRA = for | | | | | | | |
| 9 | 103036_at | Cluster incl M38700 Thyroid autoantigen 70 kDa/cds = (0,1778)/gb = M38700/gi = 198636/ug = Mm.16147/len = 1944/ | 108.8 | P | 123.8 | P | 95.3 | P | 161.9 |
| | | STRA = for | | | | | | | |
| 9 | 103342_at | Cluster Incl U78103: Embryonic ectoderm development/cds = (453,1778)/gb = U76103/gi = 1710987/ug = Mm 18203/ | 341.1 | P | 286 | P | 196.3 | P | 253.9 |
| | | len = 2040/STRA = for | | | | | | | |
| 9 | 103402_at | Cluster Incl AI848522: UI-M-AP1-agf-e-06-0-UI s2 Mus musculus cDNA, 3 end/clone = UI-M-AP1-agf-e-06-0-UI/ | 283.1 | P | 306.6 | P | 218.9 | P | 202 |
| | | clone_end = 3/gb = AI848522/gi = 5492428/ug = Mm.18761/len = 425/STRA = for | | | | | | | |
| 9 | 103406_at | Cluster Incl AI853967 UI-M-BH0-aiv-e-02-0-UI s1 Mus musculus cDNA, 3 end/clone = UI-M-BH0-aiv-e-02-0-UI/ | 341.8 | P | 376.8 | P | 137.5 | P | 221.2 |
| | | clone_end = 3/gb = AI853967/gi = 5497873/ug = Mm 18798/len = 434/STRA = for | | | | | | | |
| 9 | 103418_at | Cluster Incl AW122092: UI-M-BH2.2-aoo-g-10-0-UI s1 Mus musculus cDNA, 3 end/clone = UI-M-BH2 2-aoo-g-10-0-UI/ | 422.2 | P | 348.2 | P | 235.7 | P | 238.6 |
| | | clone_end = 3/gb = AW122092/gi = 6097499/ug = Mm.18876/len = 451/STRA = for | | | | | | | |
| 9 | 103852_at | Cluster Incl AI848207 UI-M-AH1-agq-g-08-0-UI s1 Mus musculus cDNA, 3 end/clone = UI-M-AH1-agq-g-08-0-UI/ | 201.7 | P | 201.2 | P | 119.9 | P | 159.8 |
| | | clone_end = 3/gb = AI848207/gi = 5492118/ug = Mm 21064/len = 455/STRA = for | | | | | | | |
| 9 | 103873_i_at | Cluster Incl AA684456: vm64c06.s1 Mus musculus cDNA, 5 end/clone = IMAGE-1003018/clone_end = 5/gb = AA684456/ | 221.8 | P | 232.6 | P | 155.6 | P | 129.6 |
| | | gi = 2671042/ug = Mm.21173/len = 217/STRA = for | | | | | | | |
| 9 | 103876_at | Cluster Incl AA760339: vv75c06.r1 Mus musculus cDNA, 5 end/clone = IMAGE-1228234/clone_end = 5/gb = AA760339/ | 945.6 | P | 557.5 | P | 517.2 | P | 466.9 |
| | | gi = 2808133/ug = Mm.21181/len = 552/STRA = for | | | | | | | |
| 9 | 103881_at | Cluster Incl AA691185: vt34b11.r1 Mus musculus cDNA, 5 end/clone = IMAGE-1184957/clone_end = 5/gb = AA691185/ | 303.1 | P | 362.4 | P | 271.4 | P | 241.2 |
| | | gi = 2692121/ug = Mm.212/len = 184/STRA = for | | | | | | | |
| 9 | 103886_at | Cluster Incl AI850356: UI-M-BG1-aig-a-12-0-UI s1 Mus musculus cDNA, 3 end/clone = UI-M-BG1-aig-a-12-0-UI/ | 552.7 | P | 519.3 | P | 412.2 | P | 341.1 |
| | | clone_end = 3/gb = AI850356/gi = 5494262/ug = Mm.21251/len = 443/STRA = for | | | | | | | |
| 9 | 104038_at | Cluster Incl AI843511 UI-M-AO1-aeh-b-11-0-UI a1 Mus musculus cDNA, 3 end/clone = UI-M-AO1-aeh-b-11-0-UI/ | 500.9 | P | 494.9 | P | 247.3 | P | 257.1 |
| | | clone_end = 3/gb = AI843511/gi = 5477724/ug = Mm.21451/len = 459/STRA = for | | | | | | | |
| 9 | 104052_at | Cluster Incl AI840921: UI-M-AMO-adm-f-12-0-UI s1 Mus musculus cDNA, 3 end/clone = UI-M-AM0-adm-f-12-0-UI/ | 275.4 | P | 252.6 | P | 261.1 | P | 178.7 |
| | | clone_end = 3/gb = AI840921/gi = 5475134/ug = Mm.21520/len = 204/STRA = for | | | | | | | |
| 9 | 104077_at | Cluster Incl AW121992: UI-M-BH2.3-aoj-f-11-0-UI s1 Mus musculus cDNA, 3 end/clone = UI-M-BH2.3-aoj-f-11-0-UI/ | 299.5 | P | 422.9 | P | 305.6 | P | 240 |
| | | clone_end = 3/gb = AW121992/gi = 6097398/ug = Mm 21705/len = 477/STRA = for | | | | | | | |
| 9 | 104090_at | Cluster Incl AA657164: vr26f01 r1 Mus musculus cDNA, 5 end/clone = IMAGE-1121785/clone_end = 5/gb = AA657164/ | 243.1 | P | 269.8 | P | 186.5 | P | 204.9 |
| | | gi = 2593318/ug = Mm.21803/len = 376/STRA = for | | | | | | | |
| 9 | 104126_at | Cluster Incl AI854864: UI-M-BHO-akb-e-01-0-UI a1 Mus musculus cDNA, 3 end/clone = UI-M-BHO-akb-e-01-0-UI/ | 253.6 | P | 295.9 | P | 230.9 | P | 248.2 |
| | | clone_end = 3/gb = AI854864/gi = 5498770/ug = Mm.22031/len = 197/STRA = for | | | | | | | |
| 9 | 104134_at | Cluster Incl Y17851: Mus musculus mRNA for ganglioside-induced differentiation associated protein 2/cds = (0,223)/ | 190.2 | P | 184.8 | P | 216.4 | P | 157.3 |
| | | gb = Y17851/gi = 3378455/ug = Mm.22080/len = 197/STRA = for | | | | | | | |
| 9 | 104305_at | Cluster Incl AW120679: UI-M-BH2.3-anz-c-02-0-UI s1 Mus musculus cDNA, 3 end/clone = UI-M-BH2.3-anz-c-02-0-UI/ | 498.7 | P | 385.2 | P | 268.5 | P | 260.9 |
| | | clone_end = 3/gb = AW120679/gi = 6095977/ug = Mm.22363/len = 454/STRA = for | | | | | | | |
| 9 | 104378_at | Cluster Incl L4851: Paraoxonase 2/cds = (24,1088)/gb = L48514/gi = 1333637/ug = Mm.22581/len = 1179/STRA = for | 186.8 | P | 204.8 | P | 171.4 | P | 158.2 |
| 9 | 104527_at | Cluster Incl D13803: Rad51 homolog (S cerevisiae)/cds = (188,1207)/gb = D13803/gi = 303702/ug = Mm.231/len = 1408/ | 251.3 | P | 167.9 | P | 206.1 | P | 233.5 |
| | | STRA = for | | | | | | | |
| 9 | 104544_at | Cluster Incl AA675604: vr73b04.s1 Mus musculus cDNA, 5 end/clone = IMAGE-1134319/clone_end = 5/gb = AA675604/ | 209.3 | P | 276.8 | P | 284.2 | P | 254.8 |
| | | gi = 2652841/ug = Mm.23690/len = 469/STRA = for | | | | | | | |
| 9 | 104565_at | Cluster Incl AI847561: UI-M-AM1-afv-g-05-0-UI s1 Mus musculus cDNA, 3 end/clone = UI-M-AM1-afv-g-05-0-UI/ | 449.1 | P | 359.8 | P | 317.1 | P | 271.1 |
| | | clone_end = 3/gb = AI847561/gi = 5491467/ug = Mm.23861/len = 370/STRA = for | | | | | | | |
| 9 | 104586_at | Cluster Incl AI842878: UI-M-AN1-afb-e-0-UI.s1 Mus musculus cDNA, 3 end/clone = UI-M-AN1-afb-e-12-0-UI/ | 303.4 | P | 261.2 | P | 159.4 | P | 180.8 |
| | | clone_end = 3/gb = AI842878/gi = 5477091/ug = Mm.23985/len = 162/STRA = for | | | | | | | |
| 9 | 104634_at | Cluster Incl AA874589: vx78a06 r1 Mus musculus cDNA, 5 end/clone = IMAGE-1281298/clone_end = 5/gb = AA874589/ | 734.5 | P | 723.5 | P | 435.1 | P | 560.9 |
| | | gi = 2979278/ug = Mm.24345/len = 559/STRA = for | | | | | | | |
| 9 | 104735_at | Cluster Incl AI842065: UI-M-AN1-afg-a-10-0-UI.s1 Mus musculus cDNA, 3 end/clone = UI-M-AN1-afg-a-10-0-UI/ | 393 | P | 399.9 | P | 278.2 | P | 187.7 |
| | | clone_end = 3/gb = AI842065/gi = 5476278/ug = Mm.24647/len = 444/STRA = for | | | | | | | |
| 9 | 104740_at | Cluster Incl AW125318: UI-M-BH2.1-apy-a-04-0-UI.s1 Mus musculus cDNA, 3 end/clone = UI-M-BH2.1-apy-a-04-0-UI/ | 244.1 | P | 173.9 | P | 100.5 | P | 144.6 |
| | | clone_end = 3/gb = AW125318/gi = 6100848/ug = Mm.24671/len = 405/STRA = for | | | | | | | |
| 9 | 104766_at | Cluster Incl AI851198: UI-M-BH0-ajx-e-09-0-UI.s1 Mus musculus cDNA, 3 end/clone = UI-M-BH0-ajx-e-09-0-UI/ | 706.8 | P | 900 | P | 668.6 | P | 614.1 |
| | | clone_end = 3/gb = AI851198/gi = 5495104/ug = Mm.24873/len = 193/STRA = rev | | | | | | | |
| 9 | 94346_at | Cluster Incl AI853721: UI-M-BH0-ajq-e-02-0-UI.s1 Mus musculus cDNA, 3 end/clone = UI-M-BH0-ajq-e-02-0-UI/ | 192.2 | P | 148.8 | P | 162.4 | P | 140 |
| | | clone_end = 3/gb = AI853721/gi = 5497592/ug = Mm.25154/len = 203/STRA = for | | | | | | | |
| 9 | 94966_at | Cluster Incl Z11911: Glucose-6-phosphate dehydrogenase X-linked/cds = (52,1599)/gb = Z11911/gi = 51113/ug = Mm.27210/ | 893.3 | P | 984.4 | P | 570.9 | P | 668.7 |
| | | len = 2322/STRA = for | | | | | | | |
| 9 | 94967_at | Cluster Incl AI851365: UI-M-BH0-akh-e-08-0-UI.s1 Mus musculus cDNA, 3 end/clone = UI-M-BH0-akh-e-08-0-UI/ | 443.1 | P | 400.1 | P | 376.7 | P | 307.3 |
| | | clone_end = 3/gb = AI851365/gi = 5495271/ug = Mm.27216/len = 481/STRA = for | | | | | | | |
| 9 | 94982_f_at | Cluster Incl AI852470: UI-M-BH0-alp-f-08-0-UI.s1 Mus musculus cDNA, 3 end/clone = UI-M-BH0-alp-f-08-0-UI/ | 338 | P | 335.9 | P | 244.1 | P | 215.4 |
| | | clone_end = 3/gb = AI852470/gi = 5496376/ug = Mm.27278/len = 422/STRA = for | | | | | | | |
| 9 | 94989_at | Cluster Incl AI848984: UI-M-AJ1-ahd-d-06-0UI.s1 Mus musculus cDNA, 3 end/clone = UI-M-AJ1-ahd-d-06-0-UI/ | 1071.6 | P | 1114.9 | P | 788.8 | P | 921 |
| | | clone_end = 3/gb = AI848984/gi = 5492890/ug = Mm.27311/len = 476/STRA = for | | | | | | | |
| 9 | 94998_at | Cluster Incl AI838452: UI-M-AJ0-aba-e-08-0-UI.s1 Mus musculus cDNA, 3 end/clone = UI-M-AJ0-aba-e-08-0-UI/ | 186.4 | P | 192 | P | 138.4 | P | 118.7 |
| | | clone_end = 3/gb = AI838452/gi = 5472665/ug = MM.27348/len = 466/STRA = for | | | | | | | |
| 9 | 95003_at | Cluster Incl AA80275: vv99ho2.r1 Mus musculus cDNA, 5 end/clone = IMAGE-1230579/clone_end = 5/gb = AA880275/ | 488.3 | P | 439 | P | 282.5 | P | 365.1 |
| | | gi = 2989258/ug = Mm.27375/len = 382/STRA = for | | | | | | | |
| 9 | 95018_r_at | Cluster Incl AA607053: vm95e01.r1 Mus musculus cDNA, 5 end/clone = IMAGE-1006008/clone_end = 5/gb = AA607053/ | 504.4 | P | 484.4 | P | 225.7 | P | 317.6 |
| | | gi = 2455946/ug = Mm.27454/len = 178/STRA = for | | | | | | | |
| 9 | 95614_at | Cluster Incl AI852086: UI-M-BH0-aja-g-03-0-UI.s1 Mus musculus cDNA, 3 end/clone = UI-M-BH0-aja-g-03-0-UI/ | 492.1 | P | 497.9 | P | 343.7 | P | 391.4 |
| | | clone_end = 3/gb = AI852086/gi = 5495992/ug = Mm.28003/len = 439/STRA = for | | | | | | | |
| 9 | 96158_at | Cluster Incl AW123477: UI-M-BH2.1-apj-g-12-0-UI.s1 Mus musculus cDNA, 3 end/clone = UI-M-BH2.1-apj-g-12-0-UI/ | 450.2 | P | 492.3 | P | 317.3 | P | 296.9 |
| | | clone_end = 3/gb = AW123477/gi = 6099007/ug = Mm.28332/len = 423/STRA = for | | | | | | | |
| 9 | 96168_at | Cluster Incl AI591702: vq88c03.y1 Mus musculus cDNA, 5 end/clone = IMAGE-1109380/clone_end = 5/gb = AI591702/ | 354.5 | P | 412.5 | P | 333.7 | P | 157.2 |
| | | gi = 4800750/ug = Mm.28386/len = 266/STRA = for | | | | | | | |
| 9 | 96743_at | Cluster Incl AA958560: ua14109.r1 Mus musculus cDNA, 5 end/clone = IMAGE-1346729/clone_end = 5/gb = AA958560/ | 789 | P | 678.6 | P | 452.4 | P | 497.2 |
| | | gi = 3124790/ug = Mm.28938/len = 292/STRA = for | | | | | | | |
| 9 | 97327_at | Cluster Incl L26320: Flap structure specific endonuclease 1/cds = (103,1239)/gb = L26320/gi = 499648/ug = Mm.2952/ | 444.2 | P | 437.6 | P | 217.4 | P | 346.9 |
| | | len = 2033/STRA = for | | | | | | | |
| 9 | 97329_at | Cluster Incl AW124061: UI-M-BH2.1-apn-f-03-0-UI.s1 Mus musculus cDNA, 3 end/clone = UI-M-BH2.1-apn-f-03-0-UI/ | 235.3 | P | 262.3 | P | 216.5 | P | 193 |
| | | clone_end = 3/gb = AW124061/gi = 6099591/ug = Mm.29529/len = 437/STRA = for | | | | | | | |
| 9 | 97411_at | Cluster Incl L11316: Ect2 oncogene/cds = (530,2746)/gb = L11316/gi = 293331/ug = Mm 2995/len = 3920/STRA = for | 369 | P | 341.2 | P | 159.3 | P | 210.9 |
| 9 | 97412_at | Cluster Incl AI843157: UI-M-AK1-aet-a-08-0-UI.s1 Mus musculus cDNA, 3 end/clone = UI-M-AK1-aet-a-08-0-UI/ | 623.7 | P | 673.9 | P | 387.1 | P | 387.2 |
| | | clone_end = 3/gb = AI843157/gi = 5477370/ug = Mm 29952/len = 443/STRA = for | | | | | | | |
| 9 | 97425_at | Cluster Incl AI840191: UI-M-AO0-acc-g-09-0-UI.s1 Mus musculus cDNA, 3 end/clone = UI-M-AO0-acc-g-09-0-UI/ | 597.6 | P | 409.1 | P | 380.5 | P | 330.6 |
| | | clone_end = 3/gb = AI840191/gi = 5474404/ug = Mm.30015/len = 423/STRA = for | | | | | | | |
| 9 | 97916_at | Cluster Incl AI116222: uc18c11.r1 Mus musculus cDNA, 5 end/clone = IMAGE-1398356/clone_end = 5/gb = AI116222/ | 403.8 | P | 404.6 | P | 443.4 | P | 414 |
| | | gi = 3516546/ug = Mm30116/len = 321/STRA = rev | | | | | | | |
| 9 | 97922_at | Cluster Incl AW045597: UI-M-BH1-alg-a-01-0-UIs1 Mus musculus cDNA, 3 end/clone = UI-M-BH1-alg-a-01-0-UI/ | 841.9 | P | 783 | P | 731.5 | P | 734.2 |
| | | clone_end = 3/gb = AW045597/gi = 5906121/ug = Mm30166/len = 306/STRA = for | | | | | | | |
| 9 | 98451_at | Cluster Incl AI843164: UI-M-AK1-aet-b-03-0-UIs1 Mus musculus cDNA, 3 end/clone = UI-M-AK1-aet-b-03-0-UI/ | 229.8 | P | 188.8 | P | 140.2 | P | 134.6 |
| | | clone_end = 3/gb = AI843164/gi = 5477377/ug = Mm34635/len = 375/STRA = rev | | | | | | | |
| 9 | 98463_at | Cluster Incl AW124186: UI-M-BH2.1-aph-a-05-0-UIs1 Mus musculus cDNA, 3 end/clone = UI-M-BH2.1-aph-a-05-0-UI/ | 612.9 | P | 640.6 | P | 704.8 | P | 603.2 |
| | | clone_end = 3/gb = AW124186/gi = 6099716/ug = Mm34803/len = 237/STRA = for | | | | | | | |
| 9 | 98996_at | Cluster Incl L29480 Mus musculus serine/threonine kinase (sak-b) mRNA, complete cds/cds = (205,1599)/gb = L29480/ | 415.5 | P | 349.9 | P | 186.1 | P | 197.5 |
| | | gi = 487871/ug = Mm3794/len = 1600/STRA = for | | | | | | | |
| 9 | 99440_at | Cluster Incl Y07686 M musculus mRNA for NfiB3-protein, splice vanant (exon 9, 10 and 11 missing)/cds = (531,1793)/ | 1294.3 | P | 1827.6 | P | 972.7 | P | 907.3 |
| | | gb = Y07686/gi = 1524158/ug = Mm4025/len = 2514/STRA = rev | | | | | | | |
| 9 | 99457_at | Cluster Incl X82786 Antigen identified by monoclonal antibody Ki 67/cds = (345,9161)/gb = X82786/gi = 1177527/ | 1053.9 | P | 846.7 | P | 490.4 | P | 444.4 |
| | | ug = Mm4078/len = 9471/STRA = for | | | | | | | |
| 9 | 99962_at | Cluster Incl D12644: Kinesin heavy chain member 2/cds = (504,2654)/gb = D12644/gi = 220467/ug = Mm4415/len = 3532/ | 112.2 | P | 135.9 | P | 63.7 | P | 41.5 |
| | | STRA = for | | | | | | | |
| 9 | 100033_at | Cluster Incl X81143 MutS homolog 2 (E. coli)/cds = (39,2846)/gb = X81143/gi = 534944/ug = Mm4619/len = 3079/STRA = for | 505.7 | P | 484.7 | P | 426.9 | P | 416.5 |
| 9 | 100460_at | Cluster Incl L49502: TPR-containing, SH2-binding phosphoprotein/cds = (134,3655)/gb = L49502/gi = 1236238/ug = Mm489/ | 248.9 | P | 256.8 | P | 154.7 | P | 175.2 |
| | | len = 4201/STRA = rev | | | | | | | |
| 9 | 100892_at | Cluster Incl AI850186 UI-M-BG1-aid-h-04-0-UIs1 Mus musculus cDNA, 3 end/clone = UI-M-BG1-aid-h-04-0-UI/ | 200.7 | P | 186.1 | P | 151.5 | P | 134 |
| | | clone_end = 3/gb = AI850186/gi = 5494092/ug = Mm5545/len = 271/STRA = for | | | | | | | |
| 9 | 100979_at | Cluster Incl AW209763 ul46g10 y1 Mus musculus cDNA, 5 end/clone = IMAGE-2101410/clone_end = 5/gb = AW209763/ | 263.4 | P | 190.7 | P | 130.6 | P | 142.9 |
| | | gi = 6515703/ug = Mm6703/len = 440/STRA = for | | | | | | | |
| 9 | 101367_at | Cluster Incl U60312: Dynactin 1/cds = (19,3864)/gb = U60312/gi = 2104494/ug = Mm6919/len = 4024/STRA = for | 513.3 | P | 476.5 | P | 493.3 | P | 310.2 |
| 9 | 101439_at | Cluster Incl AW122716 UI-M-BH22-aot-d-03-0-UIs1 Mus musculus cDNA, 3 end/clone = UI-M-BH22-aot-d-03-0-UI/ | 337.9 | P | 300.8 | P | 250 | P | 234.5 |
| | | clone_end = 3/gb = AW122716/gi = 6098246/ug = Mm7760/len = 409/STRA = for | | | | | | | |
| 9 | 102883_at | Cluster Incl AW107702 um33b01 x1 Mus musculus cDNA, 3 end/clone = IMAGE-2236297/clone_end = 3/gb = AW107702/ | 161.6 | P | 179.9 | P | 78.2 | P | 67.3 |
| | | gi = 6078502/ug = Mm1506/len = 548/STRA = for | | | | | | | |
| 9 | 104002_at | Cluster Incl AI153693 vz91d04 r1 Mus musculus cDNA, 5 end/clone = IMAGE-1344583/clone_end = 5/gb = AI153693/ | 86.6 | P | 81.5 | P | 106.3 | P | 29.6 |
| | | gi = 3682162/ug = Mm26595/len = 400/STRA = for | | | | | | | |
| 9 | 104030_at | Cluster Incl AI848841 UI-M-AJ1-ahb-c-08-0-UIs1 Mus musculus cDNA, 3 end/clone = UI-M-AJ1-ahb-c-08-0-UI/ | 944.1 | P | 1157.6 | P | 903.3 | P | 712.2 |
| | | clone_end = 3/gb = AI848841/gi = 5492747/ug = Mm27042/len = 249/STRA = for | | | | | | | |
| 9 | 104644_at | Cluster Incl D12646 Kinesin heavy chain member 4/cds = (731,4426)/gb = D12646/gi = 563772/ug = Mm3130/len = 4706/ | 229.1 | P | 191.6 | P | 110 | P | 137.8 |
| | | STRA = for | | | | | | | |
| 9 | 92526_f_at | Cluster Incl AW122114 UI-M-BH22-aor-a-11-0-UIs1 Mus musculus cDNA, 3 end/clone = UI-M-BH22-aor-a-11-0-UI/ | 74.2 | P | 130.4 | P | 65.8 | P | 81.8 |
| | | clone_end = 3/gb = AW122114/gi = 6097531/ug = Mm4285/len = 271/STRA = for | | | | | | | |
| 9 | 93236_s_at | Cluster Incl M13352 Thymidylate synthase/cds = (0,923)/gb = M13352/gi = 202049/ug = Mm5879/len = 924/STRA = for | 429.9 | P | 363 | P | 238.6 | P | 268.7 |
| 9 | 93859_at | Cluster Incl AI875598: uk50c11 x1 Mus musculus cDNA, 3 end/clone = IMAGE-1972436/clone_end = 3/gb = AI875598/ | 270.5 | P | 166.3 | P | 165.9 | P | 192.4 |
| | | gi = 5549647/ug = Mm87729/len = 513/STRA = rev | | | | | | | |
| 9 | 95914_at | Cluster Incl AA177382: mt29b12.r1 Mus musculus cDNA, 5 end/clone = IMAGE-622463/clone_end = 5/gb = AA177382/ | 144.1 | P | 175.2 | P | 112.1 | P | 121.7 |
| | | gi = 1758617/ug = Mm24464/len = 542/STRA = for | | | | | | | |
| 9 | 96594_at | Cluster Incl AA615831 vo90h05 r1 Mus musculus cDNA, 5 end/clone = IMAGE-1066425/clone_end = 5/gb = AA615831/ | 774.8 | P | 552.9 | P | 508.3 | P | 450.5 |
| | | gi = 2503059/ug = Mm30652/len = 257/STRA = for | | | | | | | |
| 9 | 97164_at | Cluster Incl C77566 C77566 Mus musculus cDNA, 3 end/clone = J0033G09/clone_end = 3/gb = C77566/gi = 2517896/ | 461.4 | P | 454.5 | P | 344.4 | P | 367 |
| | | ug = Mm35516/len = 616/STRA = rev | | | | | | | |
| 9 | 99366_at | Cluster Incl AI553536: vw39e06.x1 Mus musculus cDNA, 3 end/clone = IMAGE-1246210/clone_end = 3/gb = AI553536/ | 291.5 | P | 392.9 | P | 193.4 | P | 226.7 |
| | | gi = 4485899/ug = Mm5675/len = 408/STRA = rev | | | | | | | |
| 9 | 102144_f_at | Cluster Incl AW048189 UI-M-BH1-amc-d-12-0-UIs1 Mus musculus cDNA, 3 end/clone = UI-M-BH1-amc-d-12-0-UI/ | 248.3 | P | 249.7 | P | 204.7 | P | 199.1 |
| | | clone_end = 3/gb = AW048189/gi = 5908718/ug = Mm44453/len = 141/STRA = rev | | | | | | | |
| 9 | 93992_at | Cluster Incl AW123342: UI-M-BH2.1-apf-d-04-0-UIs1 Mus musculus cDNA, 3 end/clone = UI-M-BH2.1-apf-d-04-0-UI/ | 282 | P | 264.3 | P | 173.2 | P | 55.6 |
| | | clone_end = 3/gb = AW123342/gi = 6098872/ug = Mm21749/len = 405/STRA = for | | | | | | | |
| 9 | 95447_at | Cluster Incl AA683761 vr04e08 r1 Mus musculus cDNA, 5 end/clone = IMAGE-1110854/clone_end = 5/gb = AA683761/ | 377.1 | P | 378.8 | P | 278.2 | P | 290.4 |
| | | gi = 2670347/ug = Mm24601/len = 335/STRA = for | | | | | | | |
| 9 | 96002_at | Cluster Incl AW123936: UI-M-BH2.3-aqc-c-01-0-UIs1 Mus musculus cDNA, 3 end/clone = UI-M-BH2.3-aqc-c-01-0-UI/ | 267.9 | P | 247.5 | P | 212.2 | P | 288 |
| | | clone_end = 3/gb = AW123936/gi = 6099466/ug = Mm25331/len = 368/STRA = for | | | | | | | |
| 9 | 96654_at | Cluster Incl AI852916: UI-M-BH0-aiz-b-03-0-UIs1 Mus musculus cDNA, 3 end/clone = UI-M-BH0-aiz-b-03-0-UI/ | 67.1 | P | 61.1 | P | 26 | P | 48 |
| | | clone_end = 3/gb = AI852916/gi = 5496822/ug = Mm27339/len = 392/STRA = for | | | | | | | |
| 9 | 97261_at | Cluster Incl AF055664: Heat shock protein, DNAJ-like 2/cds = (91,1284)/gb = AF055664/gi = 3025863/ug = Mm27897/ | 668.9 | P | 549.9 | P | 491.3 | P | 274.3 |
| | | len = 2242/STRA = for | | | | | | | |
| 9 | 94447_at | Cluster Incl AA726837 vu94h01 r1 Mus musculus cDNA, 5 end/clone = IMAGE-1209841/clone_end = 5/gb = AA726837/ | 473.2 | P | 200.6 | P | 100.4 | P | 74.2 |
| | | gi = 2744544/ug = Mm26780/len = 654/STRA = for | | | | | | | |
| 9 | 95075_at | Cluster Incl AW229141 up29a12 y1 Mus musculus cDNA, 5 end/clone = IMAGE-2655742/clone_end = 5/gb = AW229141/ | 294.8 | P | 273.8 | P | 185.5 | P | 203.3 |
| | | gi = 6558373/ug = Mm29200/len = 287/STRA = for | | | | | | | |
| 9 | 95712_at | Cluster Incl AW045261 UI-M-BH1-akn-a-07-0-UI s1 Mus musculus cDNA, 3 end/clone = UI-M-BH1-akn-a-07-0-UI/ | 231.2 | P | 212.3 | P | 154.9 | P | 133.1 |
| | | clone_end = 3/gb = AW045261/gi = 5905790/ug = Mm29709/len = 301/STRA = for | | | | | | | |
| 9 | 99149_at | Cluster Incl AI851230 UI-M-BH0-ajx-h-10-0-UIs1 Mus musculus cDNA, 3 end/clone = UI-M-BH0-ajx-h-10-0-UI/ | 369.3 | P | 304.6 | P | 97.5 | P | 160.1 |
| | | clone_end = 3/gb = AI851230/gi = 5495136/ug = Mm41535/len = 416/STRA = for | | | | | | | |
| 9 | 99616_s_at | Cluster Incl AI842156 UI-M-AN1-afj-e-11-0-UIs1 Mus musculus cDNA, 3 end/clone = UI-M-AN1-afj-e-11-0-UI/ | 333.3 | P | 347 | P | 203.3 | P | 299.4 |
| | | clone_end = 3/gb = AI842156/gi = 5476369/ug = Mm43017/len = 316/STRA = for | | | | | | | |
| 9 | 100093_at | Cluster Incl X69025 PCTAIRE-motif protein kinase 1/cds = UNKNOWN/gb = X69025/gi = 53610/ug = Mm4597/len = 3078/ | 1005.8 | P | 811.1 | P | 811.4 | P | 766.4 |
| | | STRA = for | | | | | | | |
| 9 | 100128_at | Cluster Incl M38724: Cell division cycle control protein 2a/cds = (24,917)/gb = M38724/gi = 192533/ug = Mm.4761/len = 963/ | 509.9 | P | 445.4 | P | 243.3 | P | 121.5 |
| | | STRA = for | | | | | | | |
| 9 | 101481_at | Cluster Incl AI852555: UI-M-BH0-aiw-g-04-0-UI.s1 Mus musculus cDNA, 3 end/clone = UI-M-BH0-aiw-g-04-0-UI/ | 133.8 | P | 179 | P | 61.4 | A | 89.7 |
| | | clone_end = 3/gb = AI852555/gi = 5496461/ug = Mm.7780/len = 351/STRA = for | | | | | | | |
| 9 | 103582_r_at | Cluster Incl AI845633 UI-M-AQ1-adx-h-07-0-UI s1 Mus musculus cDNA, 3 end/clone = UI-M-AQ1-adx-h-07-0-UI/ | 194.7 | P | 192.8 | P | 131.9 | P | 153 |
| | | clone_end = 3/gb = AI845633/gi = 5489539/ug = Mm.19799/len = 430/STRA = for | | | | | | | |
| 9 | 104373_at | Cluster Incl AW046876 UI-M-BH1-aid-c-02-0-UI s1 Mus musculus cDNA, 3 end/clone = UI-M-BH1-aki-c-02-0-UI/ | 88.9 | P | 74 | P | 55.3 | P | 57.8 |
| | | clone_end = 3/gb = AW046876/gi = 5907405/ug = Mm 22645/len = 525/STRA = for | | | | | | | |
| 9 | 104547_at | Cluster Incl J00388 mouse dihydrofolate reductase gene/cds = (0,563)/gb = J00388/gi = 193340/ug = Mm 23695/len = 564/ | 105.6 | P | 110.6 | P | 55.8 | P | 54.1 |
| | | STRA = for | | | | | | | |
| 9 | 94396_at | Cluster Incl AA647507: vq78d10 s1 Mus musculus cDNA, 5 end/clone = IMAGE-1108435/clone_end = 5/gb = AA647507/ | 499.7 | P | 428.4 | P | 357.2 | P | 412.3 |
| | | gi = 2573936/ug = Mm25709/len = 345/STRA = for | | | | | | | |
| 9 | 95523_at | Cluster Incl AI839718 UI-M-AN0-acp-h-02-0-UI s1 Mus musculus cDNA, 3 end/clone = UI-M-AN0-acp-h-02-0-UI/ | 455.2 | P | 558.1 | P | 263.2 | P | 304.1 |
| | | clone_end = 3/gb = AI839718/gi = 5473931/ug = Mm.27579/len = 244/STRA = for | | | | | | | |
| 9 | 97926_s_at | Cluster Incl U10374 Peroxisome proliferator activator receptor gamma/cds = (0,1427)/gb = U10374/gi = 507776/ | 218.7 | P | 226.3 | P | 128.2 | P | 175.6 |
| | | ug = Mm 3020/len = 1428/STRA = for | | | | | | | |
| 9 | 99917_at | Cluster Incl U52951 Mus musculus putative transcriptional regulator mEnx-1 mRNA, complete cds/cds = (134,2374)/ | 354.3 | P | 373.7 | P | 211 | P | 220.6 |
| | | gb = U52951/gi = 1279908/ug = Mm4303/len = 2653/STRA = for | | | | | | | |
| 9 | 102907_at | Cluster Incl AW125043: UI-M-BH2 1-apx-b-03-0-UI s1 Mus musculus cDNA, 3 end/clone = UI-M-BH2 1-apx-b-03-0-UI/ | 275.4 | P | 217.1 | P | 140.2 | P | 59.1 |
| | | clone_end = 3/gb = AW125043/gi = 6100573/ug = Mm158/len = 465/STRA = for | | | | | | | |
| 9 | 102934_s_at | Cluster Incl L16926 Cell division cycle control protein 25C/cds = (481,1878)/gb = L16926/gi = 451133/ug = Mm16857/ | 208.1 | P | 195.8 | P | 168.9 | P | 145 |
| | | len = 2124/STRA = for | | | | | | | |
| 9 | 103696_r_at | Cluster Incl AW047329 UI-M-BH1-amh-h-04-0-UI s1 Mus musculus cDNA, 3 end/clone = UI-M-BH1-amh-h-04-0-UI/ | 28.8 | P | 59.1 | P | 52.9 | P | 39.8 |
| | | clone_end = 3/gb = AW047329/gi = 5907858/ug = Mm.23994/len = 438/STRA = for | | | | | | | |
| 9 | 103726_at | Cluster Incl AI842264 UI-M-AI1-afp-g-06-0-UI s1 Mus musculus cDNA, 3 end/clone = UI-M-AI1-afp-g-06-0-UI/clone_end = 3/ | 445.9 | P | 352 | P | 237.8 | P | 178.1 |
| | | gb = AI842264/gi = 5476477/ug = Mm24286/len = 384/STRA = for | | | | | | | |
| 9 | 104194_at | Cluster Incl AF082567 Mus musculus hephaestin (heph) mRNA, complete cds/cds = (619,4092)/gb = AF082567/gi = 4322373/ | 107.2 | P | 84.1 | P | 108 | P | 39.2 |
| | | ug = Mm27494/len = 4804/STRA = for | | | | | | | |
| 9 | 92191_at | Cluster Incl AI255450 ui85f10 x1 Mus musculus cDNA, 3 end/clone = IMAGE-1889227/clone_end = 3/gb = AI255450/ | 118.5 | P | 96.2 | P | 99.6 | P | 80.6 |
| | | gi = 3862975/ug = Mm 34009/len = 509/STRA = for | | | | | | | |
| 9 | 95379_at | Cluster Incl AA245183 mw28g07 r1 Mus musculus cDNA, 5 end/clone = IMAGE-672060/clone_end = 5/gb = AA245183/ | 151.5 | P | 143.6 | M | 81.5 | P | 66.2 |
| | | gi = 1875900/ug = Mm 22988/len = 290/STRA = rev | | | | | | | |
| 9 | 99365_at | Cluster Incl AA734487 vt94c10 r1 Mus musculus cDNA, 5 end/clone = IMAGE-1178802/clone_end = 5/gb = AA734487/ | 387.1 | P | 367.5 | P | 310.6 | P | 325.6 |
| | | gi = 2756154/ug = Mm 5662/len = 513/STRA = rev | | | | | | | |
| 9 | 95498_at | Cluster Incl AI836029 UI-M-AQ0-aad-e-12-0-UI.s2 Mus musculus cDNA, 3 end/clone = UI-M-AQ0-aad-e-12-0-UI/ | 170.8 | P | 112.9 | P | 100.4 | P | 115 |
| | | clone_end = 3/gb = AI836029/gi = 5470207/ug = Mm 25143/len = 513/STRA = for | | | | | | | |
| 9 | 96670_at | Cluster Incl AI841295 UI-M-AM0-adu-g-05-0-UI s1 Mus musculus cDNA, 3 end/clone = UI-M-AM0-adu-g-05-0-UI/ | 281 | P | 81.6 | M | 195.8 | P | 156.2 |
| | | clone_end = 3/gb = AI841295/gi = 5475508/ug = Mm 27395/len = 441/STRA = for | | | | | | | |
| 9 | 99620_at | Cluster Incl AW060546 UI-M-BH1-ann-g-04-0-UI s1 Mus musculus cDNA, 3 end/clone = UI-M-BH1-ann-g-04-0-UI/ | 145.4 | P | 103.9 | P | 90.2 | P | 80.5 |
| | | clone_end = 3/gb = AW060546/gi = 6008297/ug = Mm 43213/len = 353/STRA = for | | | | | | | |
| 9 | 100612_at | Cluster Incl K02927 Ribonucleotide reductase M1/cds = (81,2459)/gb = K02927/gi = 200764/ug = Mm 656/len = 2902/ | 690.5 | P | 634.9 | P | 474 | P | 328.5 |
| | | STRA = for | | | | | | | |
| 9 | 103385_at | Cluster Incl U64033 Mus musculus Tera (Tera) mRNA, complete cds/cds = (81,914)/gb = U64033/gi = 1575504/ | 359.9 | P | 160.1 | P | 118.6 | P | 134.3 |
| | | ug = Mm.18637/len = 2720/STRA = for | | | | | | | |
| 9 | 95032_at | Cluster Incl AA856349: vw99g06 r1 Mus musculus cDNA, 5 end/clone = IMAGE-1263130/clone_end = 5/gb = AA856349/ | 376.6 | P | 331.9 | P | 211.5 | P | 151.9 |
| | | gi = 2944651/ug = Mm 27529/len = 291/STRA = for | | | | | | | |
| 9 | 95620_at | Cluster Incl AW120882 UI-M-BH2 3-aoa-h-10-0-UI.s1 Mus musculus cDNA, 3 end/clone = UI-M-BH2 3-aoa-h-10-0-UI/ | 124.3 | P | 107.2 | P | 61.8 | P | −7 |
| | | clone_end = 3/gb = AW120882/gi = 6096215/ug = Mm 2804/len = 327/STRA = for | | | | | | | |
| 9 | 97345_at | Cluster Incl AI849333 UI-M-AJ1-aha-e-03-0-UI.s1 Mus musculus cDNA, 3 end/clone = UI-M-AJ1-ahn-e-03-0-UI/ | 310.7 | P | 325.2 | P | 221.8 | P | 240.2 |
| | | clone_end = 3/gb = AI849333/gi = 5493239/ug = Mm.29613/len = 548/STRA = for | | | | | | | |
| 9 | 97421_at | Cluster Incl U42385: Fibroblast growth factor Inducible 16/cds = (45,1187)/gb = U42385/gi = 1353708/ug = Mm 2999/ | 346.8 | P | 273.5 | P | 130.9 | P | 132.8 |
| | | len = 2808/STRA = rev | | | | | | | |
| 9 | 100413_at | Cluster Incl AB033168 Mus musculus mRNA for nuclear protein ZAP, complete cds/cds = (101,4261)/gb = AB033168/ | 286.7 | P | 210.5 | P | 208.4 | P | 177.6 |
| | | gi = 6016841/ug = Mm 46674/len = 4698/STRA = for | | | | | | | |
| 9 | 101440_at | Cluster Incl AW047074 UI-M-BH1-alr-e-08-0-UI.s1 Mus musculus cDNA, 3 end/clone = UI-M-BH1-alr-e-08-0-UI/ | 224.4 | P | 262.5 | P | 191.9 | P | 211 |
| | | clone_end = 3/gb = AW047074/gi = 5907603/ug = Mm 778/len = 330/STRA = for | | | | | | | |
| 9 | 101835_at | Cluster Incl U10484 Mus musculus lymphoid-restricted membrane protein(Jaw1) mRNA, complete cds/cds = (151,1770)/ | 11.6 | P | 13.3 | P | −137.4 | P | −20.6 |
| | | gb = U10484/gi = 505683/ug = Mm 843/len = 2023/STRA = for | | | | | | | |
| 9 | 102646_at | Cluster Incl AW122284: UI-M-BH2 2-aos-b-02-0-UI s1 Mus musculus cDNA, 3 end/clone = UI-M-BH2-2-aos-b-02-0-UI/ | 104.3 | P | 86.3 | A | 78 | P | 84 |
| | | clone_end = 3/gb = AW122284/gi = 6097765/ug = Mm 13148/len = 336/STRA = for | | | | | | | |
| 9 | 103939_at | Cluster Incl AW121399 UI-M-BH2 2-aom-h-02-0-UI s1 Mus musculus cDNA, 3 end/clone = UI-M-BH2.2-aom-h-02-0-UI/ | 161.8 | P | 154.6 | P | 139.3 | P | 83.6 |
| | | clone_end = 3/gb = AW121399/gi = 6096732/ug = Mm 25250/len = 550/STRA = for | | | | | | | |
| 9 | 104247_at | Cluster Incl AI846291: UI-M-AK1-aez-f-04-0-UI.s1 Mus musculus cDNA, 3 end/clone = UI-M-AK1-aez-f-04-0-UI/ | 207.2 | P | 196.2 | P | 176.7 | M | 182.1 |
| | | clone_end = 3/gb = AI846291/gi = 5490197/ug = Mm.28335/len = 287/STRA = for | | | | | | | |
| 9 | 104431_at | Cluster Incl D11091 Protein Kinase C, theta/cds = (88,2211)/gb = D11091/gi = 220573/ug = Mm.2921/len = 3313/STRA = for | 199.6 | P | 139.8 | P | 167.1 | P | 122.8 |
| 9 | 92468_at | Cluster Incl AF028722 Mus musculus fetal globin inducing factor mRNA, complete cds/cds = (135,851)/gb = AF028722/ | 152.9 | P | 187.3 | P | 23.6 | A | 130.2 |
| | | gi = 4103856/ug = Mm.42088/len = 1801/STRA = for | | | | | | | |
| 9 | 98335_at | Cluster Incl X72711 Replication factor C, 140 KDa/cds = (79,3474)/gb = X72711/gi = 457741/ug = Mm 4280/len = 4679/ | 285.6 | P | 317.6 | P | 185.9 | P | 221.3 |
| | | STRA = for | | | | | | | |
| 9 | 92788_f_at | Cluster Incl Y12474 M musculus mRNA for centrin gene/cds = (0,503)/gb = Y12474/gi = 2246423/ug = Mm.12481/len = 504/ | 901.8 | P | 732.4 | P | 479 | A | 463.7 |
| | | STRA = for | | | | | | | |
| 9 | 98146_at | Cluster Incl AW120775: UI-M-BH2.3-any-f-10-0-UI.s1 Mus musculus cDNA, 3 end/clone = UI-M-BH2.3-any-f-10-0-UI/ | 302.8 | P | 267.1 | P | 220.4 | P | 222.5 |
| | | clone_end = 3/gb = AW120775/gi = 6096108/ug = Mm.35698/len = 463/STRA = for | | | | | | | |
| 9 | 103354_at | Cluster Incl Z46966: M. musculus mRNA for imogen 44/cds = (45,1199)/gb = Z46966/gi = 1272668/ug = Mm.1841/len = 1267/ | 283.1 | P | 261.6 | P | 194.5 | P | 264.7 |
| | | STRA = for | | | | | | | |
| 9 | 95586_at | Cluster Incl AF089751 Mus musculus ATP receptor P2X4 subunit mRNA, complete cds/cds = (31,1197)/gb = AF089751/ | 141.2 | P | 171.6 | A | 79 | M | 110.1 |
| | | gi = 4019303/ug = Mm 27861/len = 1944/STRA = for | | | | | | | |
| 9 | 98409_at | Cluster Incl L00923 Mouse myosin I mRNA, complete cds/cds = (196,3435)/gb = L00923/gi = 200008/ug = Mm 3390/ | 189.5 | P | 276.5 | P | 217.7 | P | 115.5 |
| | | len = 4194/STRA = for | | | | | | | |
| 9 | 98995_at | Cluster Incl X86925 M musculus mRNA for E2F-5 protein/cds = (15,1022)/gb = X86925/gi = 806571/ug = Mm.379/len = 1340/ | 165.6 | P | 95.8 | P | 42.5 | A | 60.9 |
| | | STRA = for | | | | | | | |
| 9 | 99937_at | Cluster Incl Z16406 Mesenchyme homeobox 2/cds = (163,1074)/gb = Z16406/gi = 57949/ug = Mm 4346/len = 1440/ | 103.2 | P | 119.5 | P | 5.9 | A | 30.9 |
| | | STRA = for | | | | | | | |
| 9 | 100014_at | Cluster Incl AI845038 UI-M-BG0-ahy-b-01-0-UI.s1 Mus musculus cDNA, 3 end/clone = UI-M-BG0-ahy-b-01-0-UI/ | 59.2 | P | 58.6 | P | 44.5 | A | 28.6 |
| | | clone_end = 3/gb = AI845038/gi = 5488944/ug = Mm 4557/len = 380/STRA = for | | | | | | | |
| 9 | 101444_at | Cluster Incl U83176 Gene trap ROSA 26 antisense/cds = (90,1607)/gb = U83176/gi = 1778860/ug = Mm 781/len = 1974/ | 328.7 | P | 251.3 | P | 149.7 | A | 190 |
| | | STRA = for | | | | | | | |
| 9 | 93436_i_at | Cluster Incl AI850509 UI-M-BG1-aih-h-10-0-UI s1 Mus musculus cDNA, 3 end/clone = UI-M-BG1-aih-h-10-0-UI/ | 64.3 | P | 47.4 | P | 32.3 | A | 35.1 |
| | | clone_end = 3/gb = AI850509/gi = 5494415/ug = Mm 65832/len = 453/STRA = for | | | | | | | |
| 9 | 93470_at | Cluster Incl AF012129 Mus musculus putative DNA methyltransferase (Dnmt2) mRNA, complete cds/cds = (19,1266)/ | 82.5 | P | 79.8 | P | 43.1 | A | 37 |
| | | gb = AF012129/gi = 2636723/ug = Mm 6979/len = 1691/STRA = for | | | | | | | |
| 9 | 100138_f_at | Cluster Incl X52102 M musculus p16K gene for 16 kDa protein/cds = (77,1798)/gb = X52102/gi = 53546/ug = Mm 4877/ | 244.5 | P | 210.7 | P | 124.7 | M | 196 |
| | | len = 2751/STRA = for | | | | | | | |
| 9 | 100148_at | Cluster Incl U51037 Mus musculus 11-zinc-finger transcription factor (CTCF) mRNA, complete cds/cds = (306,2516)/ | 270.8 | P | 239.1 | P | 117.2 | A | 107.7 |
| | | gb = U51037/gi = 1256409/ug = Mm 493/len = 3840/STRA = for | | | | | | | |
| 9 | 103081_at | Cluster Incl AA863928 vx15h09 r1 Mus musculus cDNA, 5 end/clone = IMAGE-1264577/clone_end = 5/gb = AA863928/ | 510.7 | P | 531.1 | P | 269.6 | M | 280.3 |
| | | gi = 2956407/ug = Mm 1717/len = 273/STRA = for | | | | | | | |
| 9 | 95561_at | Cluster Incl AW120867 UI-M-BH2 3-aoa-g-06-0-UI s1 Mus musculus cDNA, 3 end/clone = UI-M-BH2 3-aoa-g-06-0-UI/ | 118.2 | A | 123.8 | P | 42.2 | A | 95.7 |
| | | clone_end = 3/gb = AW120867/gi = 6096200/ug = Mm 27777/len = 312/STRA = for | | | | | | | |
| 9 | 98515_at | Cluster Incl AI844392 UI-M-AL1-ahp-d-12-0-UI s1 Mus musculus cDNA, 3 end/clone = UI-M-AL1-ahp-d-12-0-UI/ | 111.6 | P | 80.6 | P | 148.3 | A | 119.4 |
| | | clone_end = 3/gb = AI844392/gi = 5488298/ug = Mm 36088/len = 445/STRA = for | | | | | | | |
| 9 | 98979_at | Cluster Incl U35249 Mus musculus CDK-activating kinase assembly factor p36/MAT1, complete cds/cds = (152,1081)/ | 111.7 | P | 82.9 | A | 91.5 | P | 50.5 |
| | | gb = U35249/gi = 1015998/ug = Mm 3688/len = 1409/STRA = for | | | | | | | |
| 9 | 100955_at | Cluster Incl AA989957: ua55a07 r1 Mus musculus cDNA, 5 end/clone = IMAGE-1361364/clone_end = 5/gb = AA989957/ | 69.4 | P | 103.4 | P | 39.9 | A | 20.7 |
| | | gi = 3175321/ug = Mm 6476/len = 456/STRA = for | | | | | | | |
| 9 | 102194_at | Cluster Incl AW122332 UI-M-BH2 2-aoa-f-10-0-UI s1 Mus musculus cDNA, 3 end/clone = UI-M-BH2 2-aoa-f-10-0-UI/ | 212.6 | P | 324.3 | P | 93.1 | A | 151.5 |
| | | clone_end = 3/gb = AW122332/gi = 6097817/ug = Mm 98631/len = 303/STRA = rev | | | | | | | |
| 9 | 102293_at | Cluster Incl L03547 Early lymphoid specific transcription factor/cds = (38,1333)/gb = L03547/gi = 198286/ug = Mm 12396/ | 73.5 | P | 99.4 | A | 58.4 | P | 56.8 |
| | | len = 1550/STRA = rev | | | | | | | |
| 9 | 104451_at | Cluster Incl AI852578 UI-M-BH0-aiu-a-08-0-UI s1 Mus musculus cDNA, 3 end/clone = UI-M-BH0-aiu-a-08-0-UI/ | 248.4 | P | 421.7 | P | 215.1 | M | 231.9 |
| | | clone_end = 3/gb = AI852578/gi = 5496484/ug = Mm.29433/len = 417/STRA = for | | | | | | | |
| 9 | 93217_at | Cluster Incl AW049642 UI-M-BH1-and-g-02-0-UI s1 Mus musculus cDNA, 3 end/clone = UI-M-BH1-and-g-02-0-UI/ | 294.3 | P | 211.1 | P | 251.1 | P | 215.7 |
| | | clone_end = 3/gb = AW049642/gi = 5910171/ug = Mm 5710/len = 444/STRA = for | | | | | | | |
| 9 | 93560_at | Cluster Incl AI845882 UI-M-AO1-aeg-h-05-0-UI s1 Mus musculus cDNA, 3 end/clone = UI-M-AO1-aeg-h-05-0-UI/ | 317.6 | P | 256 | P | 245.9 | A | 187.6 |
| | | clone_end = 3/gb = AI845882/gi = 5489788/ug = Mm.20309/len = 423/STRA = for | | | | | | | |
| 9 | 94000_at | Cluster Incl X07698: Mouse mRNA for CD8 molecule Ly-3 chain/cds = (49,690)/gb = X07698/gi = 52954/ug = Mm 21764/ | 312.5 | A | 423.5 | P | 308.5 | A | 213.3 |
| | | len = 1111/STRA = for | | | | | | | |
| 9 | 96656_at | Cluster Incl AI644179: vw55g04.x1 Mus musculus cDNA, 3 end/clone = IMAGE-1247766/clone_end = 3/gb = AI644179/ | 137.7 | P | 108 | P | 18.6 | P | 18 |
| | | gi = 4722654/ug = Mm 27339/len = 269/STRA = for | | | | | | | |
| 9 | 99129_at | Cluster Incl AW124529 UI-M-BH2.1-apo-g-08-0-UI s1 Mus musculus cDNA, 3 end/clone = UI-M-BH2 1-apo-g-08-0-UI/ | 153 | P | 113.7 | P | 108.2 | P | 93.2 |
| | | clone_end = 3/gb = AW124529/gi = 6100059/ug = Mm 41043/len = 503/STRA = for | | | | | | | |
| 9 | 101108_at | Cluster Incl AF034610 Mus musculus nuclear autoantigenic sperm protein mRNA, complete cds/cds = (91,2412)/ | 338.7 | P | 89.9 | P | 10.2 | A | 85.7 |
| | | gb = AF034610/gi = 2645972/ug = Mm 7516/len = 2543/STRA = for | | | | | | | |
| 9 | 104296_at | Cluster Incl AI847802: UI-M-AK1-aev-g-02-0-UI s1 Mus musculus cDNA, 3 end/clone = UI-M-AK1-aev-g-02-0-UI/ | 137.8 | P | 140.1 | P | 90.5 | M | −7.6 |
| | | clone_end = 3/gb = AI847802/gi = 5491708/ug = Mm 22334/len = 520/STRA = for | | | | | | | |
| 9 | 104535_at | Cluster Incl AF090430 Mus musculus ATP-dependent metalloprotease FlsH1 mRNA, complete cds/cds = (172,2319)/ | 108.8 | P | 65.1 | P | 24.5 | A | −0.2 |
| | | gb = AF090430/gi = 3600099/ug = Mm 23335/len = 2654/STRA = for | | | | | | | |
| 9 | 96216_at | Cluster Incl AI848826 UI-M-AJ1-ahb-b-04-0-UI s1 Mus musculus cDNA, 3 end/clone = UI-M-AJ1-ahb-b-04-0-UI/ | 208.2 | P | 148.9 | A | 99.5 | A | 124.8 |
| | | clone_end = 3/gb = AI848826/gi = 5492732/ug = Mm 28677/len = 493/STRA = for | | | | | | | |
| 9 | 102625_f_at | Cluster Incl AF080070: Mus musculus zinc finger protein 54 (Zfp54) mRNA, complete cds/cds = (121,2115)/gb = AF080070/ | 75 | P | 50.8 | P | 17.9 | A | 34.8 |
| | | gi = 3406675/ug = Mm 12916/len = 2267/STRA = rev | | | | | | | |
| 9 | 93529_at | Cluster Incl AW125219 UI-M-BH2 1-aps-c-07-0-UI.s1 Mus musculus cDNA, 3 end/clone = UI-M-BH2 1-aps-c-07-0-UI/ | 163.2 | A | 177.8 | A | 163.3 | P | 123 |
| | | clone_end = 3/gb = AW125219/gi = 6100749/ug = Mm 19803/len = 345/STRA = for | | | | | | | |
| 9 | 93758_at | Cluster Incl AA823653: vr69c07.s1 Mus musculus cDNA, 5 end/clone = IMAGE-1125900/clone_end = 5/gb = AA823653/ | 205.8 | P | 222.1 | M | 91.4 | A | 146.4 |
| | | gi = 2893521/ug = Mm.21140/len = 554/STRA = for | | | | | | | |
| 9 | 94509_at | Cluster Incl AI851207 UI-M-BH0-ajx-f-06-0-UI s1 Mus musculus cDNA, 3 end/clone = UI-M-BH0-ajx-f-06-0-UI/clone_end = 3/ | 233.2 | P | 136.2 | P | 121.9 | A | 127.8 |
| | | gb = AI851207/gi = 5495113/ug = Mm 28968/len = 332/STRA = for | | | | | | | |
| 9 | 96902_at | Cluster Incl AW121847 UI-M-BH2 3-aoi-a-12-0-UI.s1 Mus musculus cDNA, 3 end/clone = UI-M-BH2 3-aoi-a-12-0-UI/ | 224.9 | P | 214.2 | P | 207 | A | 197.8 |
| | | clone_end = 3/gb = AW121847/gi = 6097221/ug = Mm 3012/len = 241/STRA = for | | | | | | | |
| 9 | 96924_at | Cluster Incl AI849719 UI-M-AL1-ahn-b-05-0-UI.s1 Mus musculus cDNA, 3 end/clone = UI-M-AL1-ahn-b-05-0-UI/ | 445.6 | A | 346.2 | P | 329.1 | A | 267.8 |
| | | clone_end = 3/gb = AI849719/gi = 5493625/ug = Mm 30160/len = 413/STRA = for | | | | | | | |
| 9 | 99185_at | Cluster Incl AW047026 UI-M-BH1-alr-a-02-0-UI s1 Mus musculus cDNA, 3 end/clone = UI-M-BH1-alr-a-02-0-UI/ | 204.2 | P | 168.3 | M | 118.5 | A | 189.5 |
| | | clone_end = 3/gb = AW047026/gi = 5907555/ug = Mm 41874/len = 257/STRA = for | | | | | | | |
| 9 | 100577_at | Cluster Incl M58558: Small nuclear ribonucleoprotein D1/cds = (46,405)/gb = M58558/gi = 200993/ug = Mm.603/len = 506/ | 807 | P | 775.1 | A | 549.6 | A | 643.6 |
| | | STRA = for | | | | | | | |
| 9 | 104097_at | Cluster Incl AF002823 Budding inhibited by benzimidazoles 1 (S. cerevislae) homolog/cds = (60,3236)/gb = AF002823/ | 89.9 | P | 74.5 | P | 3.2 | A | 13.9 |
| | | gi = 2150135/ug = Mm 2185/len = 3370/STRA = for | | | | | | | |
| 9 | 95544_at | Clustor Incl AI843150 UI-M-AK1-aes-h-12-0-UI s1 Mus musculus cDNA, 3 end/clone = UI-M-AK1-aes-h-12-0-UI/ | 143.6 | A | 177.9 | A | −7.6 | A | 51 |
| | | clone_end = 3/gb = AI843150/gi = 5477363/ug = Mm 27687/len = 229/STRA = for | | | | | | | |
| 9 | 99541_at | Cluster Incl AJ223293 Mus musculus mRNA for kinesin-related mitotic motor protein/cds = (0,3044)/gb = AJ223293/ | 124.7 | A | 139.1 | P | 39.1 | A | 58.8 |
| | | gi = 4160555/ug = Mm.42203/len = 4412/STRA = for | | | | | | | |
| 9 | 102303_f_at | Cluster Incl L28167: Zinc finger protein 61/cds = (927,2594)/gb = L28167/gi = 758660/ug = Mm.12832/len = 2606/STRA = for | 253.3 | P | 287.1 | P | 148.9 | A | 235.9 |
| 9 | 103685_at | Cluster Incl AI839232 UI-M-AKO-adh-d-11-0-UI.s1 Mus musculus cDNA, 3 end/clone = UI-M-AKO-adh-d-11-0-UI/ | 109.4 | P | 84.5 | P | 43.4 | A | 66.3 |
| | | clone_end = 3/gb = AI839232/gi = 5473445/ug = Mm 23897/len = 407/STRA = for | | | | | | | |
| 9 | 93228_at | Cluster Incl U25691: Helicase, lymphoid specific/cds = (39,1850)/gb = U25691/gi = 805295/ug = Mm.57223/len = 1851/STRA = for | 91.7 | P | 76.9 | A | 10.2 | A | 39.8 |
| 9 | 100306_at | Cluster Incl AI510297 vx94c04 y1 Mus musculus cDNA, 5 end/clone = IMAGE-1293606/clone_end = 5/gb = AI510297/ | 159.7 | P | 98.5 | A | 109.9 | A | 66.1 |
| | | gi = 4409202/ug = Mm 86721/len = 434/STRA = for | | | | | | | |
| 10 | 93055_at | Cluster Incl AW049376 UI-M-BH1-ane-c-05-0-UI s1 Mus musculus cDNA, 3 end/clone = UI-M-BH1-ane-c-05-0-UI/ | 194.9 | P | 185.3 | P | 108.9 | P | 149.2 |
| | | clone_end = 3/gb = AW049376/gi = 5909905/ug = Mm 15351/len = 492/STRA = for | | | | | | | |
| 10 | 93258_at | Cluster Incl M28666 Hydroxymethylbilane synthase/cds = (0,1202)/gb = M28666/gi = 200231/ug = Mm 1710/len = 1203/ | 221.9 | P | 270.4 | P | 233.2 | P | 172.7 |
| | | STRA = for | | | | | | | |
| 10 | 93496_at | Cluster Incl AI852098 UI-M-BH0-aja-h-11-0-UI s1 Mus musculus cDNA, 3 end/clone = UI-M-BH0-aja-h-11-0-UI/ | 1107.8 | P | 830.7 | P | 1161.8 | P | 683.3 |
| | | clone_end = 3/gb = AI852098/gi = 5496004/ug= Mm 19130/len= 504/STRA = for | | | | | | | |
| 10 | 93521_at | Cluster Incl AW049194 UI-M-BH1-amq-g-12-0-UI.s1 Mus musculus cDNA, 3 end/clone = UI-M-BH1-amq-g-12-0-UI/ | 847.4 | P | 803 | P | 900.6 | P | 670.9 |
| | | clone_end = 3/gb = AW049194/gi = 5909723/ug = Mm 1963/len = 272/STRA = for | | | | | | | |
| 10 | 93782_at | Cluster Incl AI844517: UI-M-AJ1-ahf-a-02-0-UI s2 Mus musculus cDNA, 3 end/clone = UI-M-AJ1-ahf-a-02-0-UI/ | 1274.5 | P | 1122.1 | P | 1244 | P | 1135.6 |
| | | clone_end = 3/gb = AI844517/gi = 5488423/ug = Mm 21281/len = 504/STRA = for | | | | | | | |
| 10 | 93831_at | Cluster Incl AI316087: uk62h02 y1 Mus musculus cDNA, 5 end/clone = IMAGE-1973619/clone_end = 5/gb = AI316087/ | 336.6 | P | 298.6 | P | 327.5 | P | 243.7 |
| | | gi = 4031354/ug = Mm 21559/len = 512/STRA = for | | | | | | | |
| 10 | 94007_at | Cluster Incl AW122469 UI-M-BH2 2-aou-d-04-0-UI s1 Mus musculus cDNA, 3 end/clone = UI-M-BH2 2-aou-d-04-0-UI/ | 3143.2 | P | 3532.2 | P | 3026.8 | P | 2950 |
| | | clone_end = 3/gb = AW122469/gi = 6097989/ug = Mm 21802/len = 224/STRA = for | | | | | | | |
| 10 | 94225_at | Cluster Incl AI844679 UI-M-AL1-ahr-f-03-0-UI s1 Mus musculus cDNA, 3 end/clone = UI-M-AL1-ahr-f-03-0-UI/clone_end = 3/ | 487.9 | P | 454.1 | P | 489 | P | 422.3 |
| | | gb = AI844679/gi = 5488585/ug = Mm.22264/len = 475/STRA = for | | | | | | | |
| 10 | 94257_at | Cluster Incl AI836117 UI-M-AQ0-aae-c-02-0-UI s2 Mus musculus cDNA, 3 end/clone = UI-M-AQ0-aae-c-02-0-UI/ | 319.2 | P | 244.5 | P | 274.5 | P | 300.8 |
| | | clone_end = 3/gb = AI836117/gi = 5470325/ug = Mm 2241/len = 289/STRA = for | | | | | | | |
| 10 | 94286_at | Cluster Incl AW050340: UI-M-BH1-ang-c-08-0-UI s1 Mus musculus cDNA, 3 end/clone = UI-M-BH1-ang-c-08-0-UI/ | 419.7 | P | 458.3 | P | 456.3 | P | 425.4 |
| | | clone_end = 3/gb = AW050340/gi = 5910869/ug = Mm 22565/len = 492/STRA = for | | | | | | | |
| 10 | 94853_at | Cluster Incl U29055 Guanine nuleotide binding protein, beta 1/cds = (39,1061)/gb = 32 U29055/gi = 868166/ug = Mm 2344/ | 594.3 | P | 764.9 | P | 690.3 | P | 642 |
| | | len = 1410/STRA = for | | | | | | | |
| 10 | 94854_g_at | Cluster Incl U29055 Guanine nucleotide binding protein, beta 1/cds = (39,1061)/gb = U29055/gi = 868166/ug = Mm 2344/ | 884.4 | P | 722.8 | P | 734.2 | P | 680.4 |
| | | len = 1410/STRA = for | | | | | | | |
| 10 | 94860_at | Cluster Incl AW121821 UI-M-BH2 3-anx-d-12-0-UI s2 Mus musculus cDNA, 3 end/clone = UI-M-BH2 3-anx-d-12-0-UI/ | 875.3 | P | 890.9 | P | 737.4 | P | 666.1 |
| | | clone_end = 3/gb = AW121821/gi = 6097189/ug = Mm 2368/len = 370/STRA = for | | | | | | | |
| 10 | 94871_r_at | Cluster Incl AW124226: UI-M-BH2 1-aph-d-10-0-UI.s1 Mus musculus cDNA, 3 end/clone = UI-M-BH2 1-aph-d-10-0-UI/ | 670 | P | 707.7 | P | 634 | P | 584.6 |
| | | clone_end = 3/gb = AW124226/gi = 6099756/ug = Mm 23755/len = 382/STRA = for | | | | | | | |
| 10 | 94895_at | Cluster Incl AW122948: UI-M-BH2. 1-aoz-c-05-0-UI s2 Mus musculus cDNA, 3 end/clone = UI-M-BH2.1-aoz-c-05-0-UI/ | 607.6 | P | 512.4 | P | 546.4 | P | 385.7 |
| | | clone_end = 3/gb = AW122948/gi = 6098478/ug = Mm 23998/len = 451/STRA = for | | | | | | | |
| 10 | 94896_at | Cluster Incl D90151 Mouse mRNA for CArG-binding factor-A, complete cds/cds = (193,1050)/gb = D90151/gi = 220362/ | 1252.7 | P | 867.9 | P | 854.4 | P | 826.5 |
| | | ug = Mm.24/len = 1595/STRA = for | | | | | | | |
| 10 | 94910_at | Cluster Incl AW120739: UI-M-BH2.3-any-c-04-0-UI.s1 Mus musculus cDNA, 3 end/clone = UI-M-BH2.3-any-c-04-0-UI/ | 230.6 | P | 233.8 | P | 189.8 | P | 163.2 |
| | | clone_end = 3/gb = AW120739/gi = 6096072/ug = Mm 24105/len = 445/STRA = for | | | | | | | |
| 10 | 95419_at | Cluster Incl M29260 Mouse histone 1-0 gene, 5 end promoter region/cds = (266,268)/gp = M29260/gi = 193849/ | 2260.5 | P | 2612.9 | P | 2323.2 | P | 2013.7 |
| | | ug = Mm 24350/len = 269/STRA = for | | | | | | | |
| 10 | 95460_at | Clustor Incl U70736 Mus musculus 38 kDa Mov34 homolog mRNA, complete cds/cds = (123,1127)/gb = U70736/ | 701.1 | P | 646.9 | P | 516.4 | P | 562.7 |
| | | gi = 2360946/ug = Mm 2472/len = 1190/STRA = for | | | | | | | |
| 10 | 96065_at | Cluster Incl D88769 Mus musculus mRNA fo latexin, complete cds/cds = (99,767)/gp = U88769/gi = 1669620/ug = Mm 2632 | 286.4 | P | 360.3 | P | 315.1 | P | 202.9 |
| | | len = 996/STRA = for | | | | | | | |
| 10 | 96606_at | Cluster Incl AB025217: Mus musculus mRNA for Sid470p, complete cds/cds = (55, 642)/gb = AB025217/gi = 5931554/ | 394.1 | P | 320.3 | P | 323.1 | P | 262.2 |
| | | ug = Mm 27065/len = 782/STRA = for | | | | | | | |
| 10 | 96626_at | Cluster Incl AI843401 UI-M-AQ1-adz-h-01-0-UI.s1 Mus musculus cDNA, 3 end/clone = UI-M-AQ1-adz-h-01-0-UI/ | 605.1 | P | 717.2 | P | 654.1 | P | 621.9 |
| | | clone_end = 3/gb = AI843401/gi = 5477614/ug = Mm 27182/len = 336/STRA = for | | | | | | | |
| 10 | 96700_r_at | Cluster Incl AW060594 UI-M-BH1-anq-d-05-0-UI.s1 Mus musculus cDNA, 3 end/clone = UI-M-BH1-anq-d-05-0-UI/ | 108.3 | P | 121.5 | P | 74.5 | P | 80.6 |
| | | clone_end = 3/gb = AW060594/gi = 6008345/ug = Mm 27560/len = 245/STRA = for | | | | | | | |
| 10 | 97272_at | Cluster Incl M99167 Mouse rearranged RNA binding protein (FLI-2) mRNA, complete cds/cds = (59,1021)/gb = M99167/ | 690.1 | P | 851.6 | P | 710.2 | P | 508.1 |
| | | gi = 193323/ug = Mm 27927/len = 1443/STRA = for | | | | | | | |
| 10 | 97312_at | Cluster Incl AB014464 Mus musculus mRNA for MGC-24v, complete cds/cds = (0,593)/gb = AB014464/gi = 3892655/ | 2001.2 | P | 1751.6 | P | 1744.5 | P | 1874.3 |
| | | ug = Mm 28083/len = 594/STRA = for | | | | | | | |
| 10 | 97818_at | Cluster Incl AI838691 UI-M-AO0-aca-g-07-0-UI.s1 Mus musculus cDNA, 3 end/clone = UI-M-AO0-aca-g-07-0-UI/ | 712.6 | P | 763.8 | P | 721.9 | P | 693.9 |
| | | clone_end = 3/gb = AI838691/gi = 5472904/ug = Mm 28196/len = 387/STRA = for | | | | | | | |
| 10 | 97843_at | Cluster Incl AI834866: UI-M-AN1-afe-a-11-0-UI s1 Mus musculus cDNA, 3 end/clone = UI-M-AN1-afe-a-11-0-UI/ | 769.4 | P | 805.1 | P | 773.1 | P | 586.8 |
| | | clone_end = 3/gb = AI834866/gi = 5469079/ug = Mm 28261/len = 488/STRA = for | | | | | | | |
| 10 | 98917_at | Clustor Incl AA823202: vw41h10r1 Mus musculus cDNA, 5 end/clone = IMAGE-1246435/clone_end = 5/gb = AA823202/ | 632.9 | P | 610.9 | P | 628.5 | P | 391.9 |
| | | gi = 2893070/ug = Mm 2862/len = 363/STRA = for | | | | | | | |
| 10 | 98929_at | Cluster Incl AI846452: UI-M-AQ1-aee-f-09-0-UI.s1 Mus musculus cDNA, 3 end/clone = UI-M-AQ1-aee-f-09-0-UI/ | 473.8 | P | 383.9 | P | 387.6 | P | 387 |
| | | clone_end = 3/gb = AI846452/gi = 5490358/ug = Mm.28664/len = 283/STRA = for | | | | | | | |
| 10 | 94562_at | Cluster Incl AI843968: UI-M-AP1-agh-e-10-0-UI s1 Mus musculus cDNA, 3 end/clone = UI-M-AP1-agh-e-10-0-UI/ | 602.8 | P | 670.7 | P | 565.5 | P | 429.1 |
| | | clone_end = 3/gb = AI843968/gi = 5478181/ug = Mm 29114/len = 334/STRA = for | | | | | | | |
| 10 | 95153_at | Cluster Incl AI845988: UI-M-AP1-agl-d-01-0-UI s1 Mus musculus cDNA, 3 end/clone = UI-M-AP1-agl-d-01-0-UI/ | 265.8 | P | 431.6 | P | 317.8 | P | 324.1 |
| | | clone_end = 3/gb = AI845988/gi = 5489894/ug = Mm 29476/len = 435/STRA = for | | | | | | | |
| 10 | 95650_at | Cluster Incl U95114 Mus musculus fertilization antigen-1 mRNA, complete cds/cds = (82,576)/gb = U95114/gi = 2098784/ | 1250.7 | P | 1018.9 | P | 1076.1 | P | 975.9 |
| | | ug = Mm.29504/len = 642/STRA = for | | | | | | | |
| 10 | 95665_at | Cluster Incl AI852087 UI-M-BH0-aja-g-04-0-UI s1 Mus musculus cDNA, 3 end/clone = UI-M-BH0-aja-g-04-0-UI/ | 396.2 | P | 350.9 | P | 312 | P | 253.1 |
| | | clone_end = 3/gb = AI852087/gi = 5495993/ug = Mm 29571/len = 260/STRA = for | | | | | | | |
| 10 | 95754_at | Cluster Incl AI838216 UI-M-AL0-abx-c-12-0-UI s1 Mus musculus cDNA, 3 end/clone = UI-M-AL0-abx-c-12-0-UI/ | 642.9 | P | 569.2 | P | 724.2 | P | 601.1 |
| | | clone_end = 3/gb = AI838216/gi = 5472429/ug = Mm 29791/len = 474/STRA = for | | | | | | | |
| 10 | 96243_f_at | Cluster Incl AW120804 UI-M-BH2 3-aoa-a-08-0-UI s1 Mus musculus cDNA, 3 end/clone = UI-M-BH2 3-aoa-a-08-0-UI/ | 335.9 | P | 348.9 | P | 341.8 | P | 217.3 |
| | | clone_end = 3/gb = AW120804/gi = 6096137/ug = Mm 29803/len = 289/STRA = for | | | | | | | |
| 10 | 96257_at | Cluster Incl AW124376 UI-M-BH2 1-ape-a-11-0-UI s1 Mus musculus cDNA, 3 end/clone = UI-M-BH2 1-ape-a-11-0-UI/ | 641.9 | P | 673 | P | 720.6 | P | 685 |
| | | clone_end = 3/gb = AW124376/gi = 6099906/ug = Mm 29822/len = 213/STRA = for | | | | | | | |
| 10 | 97488_at | Cluster Incl AI595893: uk19g08 x1 Mus musculus cDNA, 3 end/clone = IMAGE-1969502/clone_end = 3/gb = AI595893/ | 697.7 | P | 633.2 | P | 585 | P | 609.6 |
| | | gi = 4604941/ug = Mm 3094/len = 448/STRA = for | | | | | | | |
| 10 | 98586_at | Cluster Incl AW107230: um12f03 x1 Mus musculus cDNA, 3 end/clone = IMAGE-2182877/clone_end = 3/gb = AW107280/ | 376 | P | 328.6 | P | 288.9 | P | 283.2 |
| | | gi = 6078030/ug = Mm.3797/len = 574/STRA = for | | | | | | | |
| 10 | 99162_at | Cluster Incl AW122398: UI-M-BH2 2-aow-d-11-0-UI s1 Mus musculus cDNA, 3 end/clone = UI-M-BH2 2-aow-d-11-0-UI/ | 230.2 | P | 283.5 | P | 254.3 | P | 177.6 |
| | | clone_end = 3/gb = AW122398/gi = 6097899/ug = Mm 41670/len = 506/STRA = for | | | | | | | |
| 10 | 99575_at | Cluster Incl AA517835 vh82b01 r1 Mus musculus cDNA, 3 end/clone = IMAGE-893449/clone_end = 3/gb = AA517835/ | 1810.6 | P | 1520.4 | P | 1575.1 | P | 1205 |
| | | gi = 2257359/ug = Mm 4230/len = 440/STRA = for | | | | | | | |
| 10 | 100073_at | Cluster Incl AW045965 UI-M-BH1-aku-h-09-0-UI s1 Mus musculus cDNA, 3 end/clone = UI-M-BH1-aku-h-09-0-UI/ | 274.1 | P | 253.6 | P | 219.3 | P | 177.7 |
| | | clone_end = 3/gb = AW045965/gi = 5906494/ug = Mm 45225/len = 554/STRA = for | | | | | | | |
| 10 | 101096_s_at | Cluster Incl AF023482 Mus musculus HS1-associating protein (mHAX-1s) mRNA, complete cds/cds = (108,950)/ | 614.2 | P | 418.8 | P | 325.6 | P | 398.1 |
| | | gb = AF023482/gi = 2522493/ug = Mm 7401/len = 1121/STRA = for | | | | | | | |
| 10 | 101101_at | Cluster Incl Z67746 M musculus mRNA for phosphatase 2A catalytic subnit, isotype beta/cds = (0,929)/gb = Z67746/ | 690.1 | P | 572.6 | P | 628.7 | P | 606.4 |
| | | gi = 1246796/ug = Mm 7418/len = 930/STRA = for | | | | | | | |
| 10 | 101960_at | Cluster Incl AI842208 UI-M-AI1-afm-f-10-0-UI s1 Mus musculus cDNA, 3 end/clone = UI-M-AI1-afm-f-10-0-UI/clone_end = 3/ | 573.4 | P | 501.9 | P | 516.7 | P | 388.4 |
| | | gb = AI842208/gi = 5476421/ug = Mm 9257/len = 382/STRA = for | | | | | | | |
| 10 | 102019_at | Cluster Incl AA666635 vm50b08 r1 Mus musculus cDNA, 5 end/clone = IMAGE-1001655/clone_end = 5/gb = AA666835/ | 697.7 | P | 654.3 | P | 535.5 | P | 509 |
| | | gi = 2625336/ug = Mm 10076/len = 203/STRA = for | | | | | | | |
| 10 | 102039_at | Cluster Incl AI850881 UI-M-BH0-ajt-f-06-UI s1 Mus musculus cDNA, 3 end/clone = UI-M-BH0-ajt-1-06-0-UI/clone_end = 3/ | 299 | P | 317.9 | P | 310.5 | P | 253.2 |
| | | gb = AI850881/gi = 5494787/ug = Mm 10182/len = 471/STRA = for | | | | | | | |
| 10 | 103047_at | Cluster Incl AF031128 Mus musculus peroxisome membrane protein PEX2 mRNA, complete cds/cds = (501,1418)/ | 399.9 | P | 379 | P | 290.1 | P | 393.9 |
| | | gb = AF031128/gi = 2623263/ug = Mm 16453/len = 1749/STRA = for | | | | | | | |
| 10 | 103428_at | Cluster Incl AW049684 UI-M-BH1-ani-b-12-0-UI s1 Mus musculus cDNA, 3 end/clone = UI-M-BH1-ani-b-12-0-UI/ | 418.7 | P | 303.1 | P | 267.8 | P | 311.4 |
| | | clone_end = 3/gb = AW049684/gi = 5910213/ug = Mm.19000/len = 346/STRA = for | | | | | | | |
| 10 | 103848_at | Cluster Incl AW121188 UI-M-BH2.3-aok-b-04-0-UI.s1 Mus musculus cDNA, 3 end/clone = UI-M-BH2.3-aok-b-04-0-UI/ | 458.4 | P | 457.6 | P | 349.9 | P | 361 |
| | | clone_end = 3/gb = AW121188/gi = 6096521/ug = Mm.21048/len = 477/STRA = for | | | | | | | |
| 10 | 103861_s_at | Cluster Incl AA388099: vc88g07.r1 Mus musculus cDNA, 5 end/clone = IMAGE-790140/clone_end = 5/gb = AA388099/ | 384.6 | P | 291.7 | P | 239.5 | P | 266.9 |
| | | gi = 2041044/ug = Mm.21103/len = 564/STRA = for | | | | | | | |
| 10 | 104297_at | Cluster Incl AW124742 UI-M-BH2 1-aqa-d-07-0-UI s1 Mus musculus cDNA, 3 end/clone = UI-M-BH2 1-aqa-d-07-0-UI/ | 360.9 | P | 355 | P | 318.6 | P | 324 |
| | | clone_end = 3/gb = AW124742/gi = 6100272/ug = Mm.22336/len = 466/STRA = for | | | | | | | |
| 10 | 94360_at | Cluster Incl AA672650 vp57b04 r1 Mus musculus cDNA, 3 end/clone = IMAGE-1080751/clone_end = 3/gb = AA672650/ | 323.4 | P | 280.1 | P | 271.8 | P | 175.9 |
| | | gi = 2644867/ug = Mm.25263/len = 408/STRA = for | | | | | | | |
| 10 | 94362_at | Cluster Incl AI843682 UI-M-AK1-aer-c-04-0-UI.s1 Mus musculus cDNA, 3 end/clone = UI-M-AK1-aer-c-04-0-UI/ | 1353.9 | P | 1386 | P | 1126.9 | P | 1260.7 |
| | | clone_end = 3/gb = AI843682/gi = 5477895/ug = Mm 25265/len = 160/STRA = for | | | | | | | |
| 10 | 94925_at | Cluster Incl AA754865: vu57f07 r1 Mus musculus cDNA, 5 end/clone = IMAGE-1194949/clone_end = 5/gb = AA754865/ | 265.5 | P | 315 | P | 287.6 | P | 193.6 |
| | | gi = 2802063/ug = Mm.26644/len = 324/STRA = for | | | | | | | |
| 10 | 95525_at | Cluster Incl AI850793 UI-M-BG1-all-e-06-0-UI s1 Mus musculus cDNA, 3 end/clone = UI-M-BG1-all-e-06-0-UI/ | 389.6 | P | 319.8 | P | 221.1 | P | 303.8 |
| | | clone_end = 3/gb = AI850793/gi = 5494699/ug = Mm.27592/len = 517/STRA = for | | | | | | | |
| 10 | 95539_at | Cluster Incl AA798971 vv95e03.r1 Mus musculus cDNA, 5 end/clone = IMAGE-1230172/clone_end = 5/gb = AA798971/ | 230.3 | P | 183.3 | P | 214.1 | P | 187.2 |
| | | gi = 2861926/ug = Mm.27668/len = 449/STRA = for | | | | | | | |
| 10 | 96146_at | Cluster Incl D83745: Mus musculus mRNA for ANA, complete cds/cds = (173,931)/gb = D83745/gi = 3256228/ug = Mm.2823/ | 707.3 | P | 541.3 | P | 595.2 | P | 492.4 |
| | | len = 1380/STRA = for | | | | | | | |
| 10 | 96744_at | Cluster Incl AW061051 UI-M-BH1-akk-f-05-0-UI s2 Mus musculus cDNA, 3 end/clone = UI-M-BH1-akk-f-05-0-UI/ | 552 | P | 557.7 | P | 433.6 | P | 312 |
| | | clone_end = 3/gb = AW061051/gi = 6008802/ug = Mm 28950/len = 517/STRA = for | | | | | | | |
| 10 | 96754_s_at | Cluster Incl AI844751 UI-M-AL1-ahq-e-01-0-UI.s1 Mus musculus cDNA, 3 end/clone = UI-M-AL1-ahq-e-01-0-UI/ | 1226.2 | P | 1241.4 | P | 1096 | P | 954.3 |
| | | clone_end = 3/gb = AI844751/gi = 5488657/ug = Mm 28982/len = 437/STRA = for | | | | | | | |
| 10 | 96804_at | Cluster Incl AI854811 UI-M-BH0-aka-g-11-0-UI s1 Mus musculus cDNA, 3 end/clone = UI-M-BH0-aka-g-11-0-UI/ | 367.7 | P | 441.1 | P | 176 | P | 258 |
| | | clone_end = 3/gb = AI854811/gi = 5498717/ug = Mm 29203/len = 366/STRA = rev | | | | | | | |
| 10 | 97380_at | Cluster Incl AI197431 uf13e07.r1 Mus musculus cDNA, 3 end/clone = IMAGE-1851204/clone_end = 5/gb = AI197431/ | 431.8 | P | 393.5 | P | 425.5 | P | 333.4 |
| | | gi = 3750037/ug = Mm 29748/len = 331/STRA = for | | | | | | | |
| 10 | 97390_at | Cluster Incl AW125294 UI-M-BH2.1-apy-b-10-0-UI s1 Mus musculus cDNA, 3 end/clone = UI-M-BH2.1-apy-b-10-0-UI/ | 363.8 | P | 400.5 | P | 305.1 | P | 254.4 |
| | | clone_end = 3/gb = AW125294/gi = 6100824/ug = Mm 29800/len = 345/STRA = for | | | | | | | |
| 10 | 97979_at | Cluster Incl AI849126: UI-M-AJ1-agy-a-10-0-UI s1 Mus musculus cDNA, 3 end/clone = UI-M-AJ1-agy-a-10-0-UI/ | 282.4 | P | 272.1 | P | 290.4 | P | 224.3 |
| | | clone_end = 3/gb = AI849126/gi = 5493032/ug = Mm 3121/len = 489/STRA = for | | | | | | | |
| 10 | 99499_at | Cluster Incl AB023619 Mus musculus mRNA, for methyltransferase related protein, complete cds/cds = (47,853)/ | 379.6 | P | 293 | P | 305.7 | P | 301.5 |
| | | gb = AB023619/gi = 4512324/ug = Mm 41667/len = 887/STRA = for | | | | | | | |
| 10 | 99529_f_at | Cluster Incl AB025011: Mus musculus mRNA for Trif-d, complete cds/cds = (303,590)/gb = AB025011/gi = 4586406/ | 160.6 | P | 139.6 | P | 121.4 | P | 127.5 |
| | | ug = Mm.42154/len = 1078/STRA = for | | | | | | | |
| 10 | 100425_at | Cluster Incl U25685: Mus musculus protein tyrosine kinase (syk) mRNA complete cds/cds = (0,1889)/gb = U25685/ | 577.5 | P | 537.5 | P | 566.4 | P | 566.2 |
| | | gi = 818860/ug = Mm 4708/len = 1890/STRA = for | | | | | | | |
| 10 | 101944_at | Cluster Incl U89352: Mus musculus lysophospholipase I mRNA, complete cds/cds = (0,692)/gb = U89352/gi = 1864158/ | 627.3 | P | 987.1 | P | 641.7 | P | 652 |
| | | ug = Mm 95696/len = 693/STRA = for | | | | | | | |
| 10 | 101946_at | Cluster Incl AA840463 vx93d09.r1 Mus musculus cDNA, 5 end/clone = IMAGE-1282769/clone_end = 5/gb = AA840463/ | 366.8 | P | 300.7 | P | 269.3 | P | 368.4 |
| | | gi = 2916122/ug = Mm 95696/len = 430/STRA = for | | | | | | | |
| 10 | 102271_at | Cluster Incl AW125462 UI-M-BH2 2-aqm-a-11-0-UI s1 Mus musculus cDNA, 3 end/close = UI-M-BH2 2-aqm-a-11-0-UI/ | 574.8 | P | 599.7 | P | 540.1 | P | 583.5 |
| | | clone_end = 3/gb = AW125462/gi = 6100992/ug = Mm.1144/len = 439/STRA = for | | | | | | | |
| 10 | 103242_at | Cluster Incl AW123834 UI-M-BH2.1-apm-h-06-0-UI s1 Mus musculus cDNA, 3 end/clone = UI-M-BH2 1-apm-h-06-0-UI/ | 381.9 | P | 439.2 | P | 67.4 | P | 106.1 |
| | | clone_end = 3/gb = AW123834/gi = 6099364/ug = Mm.20417/len = 403/STRA = for | | | | | | | |
| 10 | 103754_at | Cluster Incl AI847826 UI-M-AM1-afu-a-09-0-UI s1 Mus musculus cDNA, 3 end/clone = UI-M-AM1-afu-a-09-0-UI/ | 635.7 | P | 388.8 | P | 483.5 | P | 321.7 |
| | | clone_end = 3/gb = AI847826/gi = 5491732/ug = Mm 24529/len = 301/STRA = for | | | | | | | |
| 10 | 104447_r_at | Cluster Incl AI595812: uj69f12.x1 Mus musculus cDNA, 3 end/clone = IMAGE-1925231/clone_end = 3/gb = AI595812/ | 69.8 | P | 52.4 | P | 58.8 | P | 30.4 |
| | | gi = 4604860/ug = Mm 29409/len = 596/STRA = for | | | | | | | |
| 10 | 92389_at | Cluster Incl AB025411: Mus musculus mRNA for Ten-m2, complete cds/cds = (112,8406)/gb = AB025411/gi = 4760777/ | 788.6 | P | 668.9 | P | 738.4 | P | 518 |
| | | ug = Mm 40599/len = 8797/STRA = rev | | | | | | | |
| 10 | 93215_at | Cluster Incl AF061346 Tumor necrosis factor induced protein 1/cds = (0,950)/gb = AF061346/gi = 3114712/ug = Mm.57088/ | 367.4 | P | 241.7 | P | 292.9 | P | 325.2 |
| | | len = 951/STRA = for | | | | | | | |
| 10 | 93430_at | Cluster Incl AF000236 Chemokine orphan receptor 1/cds = (88,1176)/gb = AF000236/gi = 2439995/ug = Mm 6522/len = 1861/ | 122.6 | P | 142.9 | P | 142.1 | P | 124.4 |
| | | STRA = for | | | | | | | |
| 10 | 93918_at | Cluster Incl AA673500 vp49g08 r1 Mus musculus cDNA, 3 end/clone = IMAGE-1080062/clone_end = 3/gb = AA673500/ | 390.5 | P | 296 | P | 355.5 | P | 273.6 |
| | | gi = 2646782/ug = Mm 89836/len = 357/STRA = for | | | | | | | |
| 10 | 94189_at | Cluster Incl AB011665 Mus musculus mRNA for BAZF, complete cds/cds = (24,1448)/gb = AB01165/gi = 3287500/ | 785.6 | P | 781.6 | P | 797.8 | P | 687.8 |
| | | ug = Mm 12930/len = 3345/STRA = for | | | | | | | |
| 10 | 95885_at | Cluster Incl AA177621 mt32e09 r1 Mus musculus cDNA, 5 end/clone = IMAGE-622792/clone_end = 5/gb = AA177621/ | 201.7 | P | 158.3 | P | 96.7 | P | 132.4 |
| | | gi = 1758855/ug = Mm 23986/len = 517/STRA = for | | | | | | | |
| 10 | 97104_g_at | Cluster Incl AF031380 Mus musculus KOI-4 gene, partial cds/cds = (0,102)/gb = AF031380/gi = 2623675/ug = Mm 32715/ | 448.2 | P | 292.5 | P | 318.8 | P | 383.4 |
| | | len = 103/STRA = rev | | | | | | | |
| 10 | 97770_s_at | Cluster Incl AA733372 vt74c06 r1 Mus musculus cDNA, 5 end/clone = IMAGE-1176874/clone_end = 5/gb = AA733372/ | 164.8 | P | 209.1 | P | 124.8 | P | 149.4 |
| | | gi = 2755039/ug = Mm 4176/len = 316/STRA = for | | | | | | | |
| 10 | 98356_at | Cluster Incl AW125421 UI-M-BH2 3-aqh-f-02-0-UI s1 Mus musculus cDNA, 3 end/clone = UI-M-BH2 3-aqh-f-02-0-UI/ | 201.9 | P | 227.5 | P | 167.2 | P | 126.9 |
| | | clone_end = 3/gb = AW125421/gi = 6100951/ug = Mm.43948/len = 570/STRA = rev | | | | | | | |
| 10 | 93567_at | Cluster Incl AW122536 UI-M-BH2 2-aox-c-06-0-UI s1 Mus musculus cDNA, 3 end/clone = UI-M-BH2 2-aox-c-06-0-UI/ | 281.7 | P | 238.5 | P | 265 | P | 167.9 |
| | | clone_end = 3/gb = AW122536/gi = 6098060/ug = Mm.20399/len = 523/STRA = for | | | | | | | |
| 10 | 97847_at | Cluster Incl AJ237847: Mus musculus mRNA for heterogeneous nuclear ribonucleoprotein G, splice variant 1/ | 354.7 | P | 329.1 | P | 327 | P | 309.2 |
| | | cds = (153,1328)/gb = AJ237847/gi = 5579010/ug = Mm 28275/len = 2074/STRA = for | | | | | | | |
| 10 | 102319_at | Cluster Incl AF062484 Mus musculus SDP8 mRNA, complete cds/cds = (64,561)/gb = AF062484/gi = 3126980/ | 282.1 | P | 19.6 | P | 152.5 | P | 33.8 |
| | | ug = Mm.10704/len = 617/STRA = for | | | | | | | |
| 10 | 95573_at | Cluster Incl AW122821: UI-M-BH2 1-aoz-c-02-0-UI s1 Mus musculus cDNA, 3 end/clone = UI-M-BH2 1-aoz-c-02-0-UI/ | 349.6 | P | 448.4 | P | 360.4 | P | 354.9 |
| | | clone_end = 3/gb = AW122821/gi = 6098351/ug = Mm 27809/len = 329/STRA = rev | | | | | | | |
| 10 | 98006_at | Cluster Incl D13546 Mus musculus mRNA for DNA polymerase alpha associated subunit, complete cds/cds = (235,2037)/ | 226.5 | P | 368.8 | P | 318.3 | P | 158.6 |
| | | gb = D13546/gi = 303658/ug = Mm 320/len = 2352/STRA = for | | | | | | | |
| 10 | 99032_at | Cluster Incl AF009246 Mus musculus ras-related protein (DEXRAS1) mRNA, complete cds/cds = (141,983)/gb = AF009246/ | 244.2 | P | 248.1 | P | 146.8 | P | 170.5 |
| | | gi = 2253712/ug = Mm 3903/len = 1623/STRA = for | | | | | | | |
| 10 | 99078_at | Cluster Incl AI839522: UI-M-ANO-acn-d-09-UI s1 Mus musculus cDNA, 3 end/clone = UI-M-ANO-acn-d-09-0-UI/ | 618.7 | P | 486.5 | P | 570.4 | P | 412.2 |
| | | clone_end = 3/gb = AI839522/gi = 5473735/ug = Mm 4022/len = 481/STRA = for | | | | | | | |
| 10 | 103681_at | Cluster Incl AW049274: UI-M-BH1-anb-d-07-0-UI s1 Mus musculus cDNA, 3 end/clone = UI-M-BH1-anb-d-07-0-UI/ | 550.1 | P | 351.8 | P | 441.8 | P | 333.1 |
| | | clone_end = 3/gb = AW049274/gi = 5909768/ug = Mm 23885/len = 471/STRA = for | | | | | | | |
| 10 | 104396_at | Cluster Incl AI019327 ub 19g08 r1 Mus musculus cDNA, 5 end/clone = IMAGE-1378238/clone_end = 5/gb = AI019327/ | 303 | P | 165.3 | P | 190 | P | 162 |
| | | gi = 3233683/ug = Mm 28617/len = 265/STRA = for | | | | | | | |
| 10 | 93680_at | Cluster Incl D89728 Mus musculus mRNA for LOK, complete cds/cds = (99,2999)/gb = D89728/gi = 2723474/ug = Mm 8235/ | 285.5 | P | 316.1 | P | 156.3 | P | 112.7 |
| | | len = 5002/STRA = for | | | | | | | |
| 10 | 94709_at | Cluster Incl D88611: Glial cells missing homolog (Drosophila), related sequence 2/cds = (241,1755)/gb = D88611/ | 235.2 | P | 292.7 | A | 217.8 | P | 179 |
| | | gi = 1769815/ug = Mm 1399/len = 3118/STRA = for | | | | | | | |
| 10 | 99338_at | Cluster Incl AA674798: vm74h03 s1 Mus musculus cDNA, 5 end/clone = IMAGE-1004021/clone_end = 5/gb = AA674798/ | 297.8 | P | 291.6 | P | 256 | P | 240.9 |
| | | gi = 2652035/ug = Mm.5300/len = 466/STRA = rev | | | | | | | |
| 10 | 93248_at | Cluster Incl AI050598 ub33h10 r1 Mus musculus cDNA, 5 end/clone = IMAGE-1379587/clone_end = 5/gb = AI050598/ | 191.5 | P | 233.2 | P | 172.9 | P | 173.6 |
| | | gi = 3299715/ug = Mm.16925/len = 487/STRA = for | | | | | | | |
| 10 | 96678_at | Cluster Incl AW260761 um87b01 y1 Mus musculus cDNA, 5 end/clone = IMAGE-2331721/clone_end = 5/gb = AW260761/ | 96 | P | 186.5 | P | 97 | P | 126.8 |
| | | gi = 6633742/ug = Mm.27427/len = 336/STRA = for | | | | | | | |
| 10 | 96148_at | Cluster Incl AA028728 mi16d09.r1 Mus musculus cDNA, 5 end/clone = IMAGE-463697/clone_end = 5/gb = AA028728/ | 181 | P | 138.3 | P | 163.2 | P | 183.4 |
| | | gi = 1494856/ug = Mm.3572/len = 1061/STRA = for | | | | | | | |
| 10 | 100570_at | Cluster Incl U27462 Mus musculus BS4 peptide mRNA, complete cds/cds = (247,2280)/gb = U27462/gi = 863013/ | 383.4 | P | 356.2 | A | 288 | P | 338.7 |
| | | ug = Mm 5856/len = 3312/STRA = for | | | | | | | |
| 10 | 103863_at | Cluster Incl AW049769 UI-M-BH1-anm-c-05-0-UI.s1 Mus musculus cDNA, 3 end/clone = UI-M-BH1-anm-c-05-0-UI/ | 671.3 | P | 852.5 | P | 778.4 | P | 619.4 |
| | | clone_end = 3/gb = AW049769/gi = 5910298/ug = Mm 21104/len = 543/STRA = rev | | | | | | | |
| 10 | 98992_at | Cluster Incl AB030483: Mus musculus mRNA for B9, complete cds/cds = (37,651)/gb = AB030483/gi = 5668700/ | 242.1 | P | 217.8 | P | 155.5 | P | 79.3 |
| | | ug = Mm 37756/len = 817/STRA = for | | | | | | | |
| 10 | 99028_at | Cluster Incl AI788534 uk46b08.x1 Mus musculus cDNA, 3 end/clone = IMAGE-1972023/clone_end = 3/gb = AI788534/ | 177 | P | 126.9 | P | 119.2 | P | 133.2 |
| | | gi = 5336250/ug = Mm.38831/len = 682/STRA = for | | | | | | | |
| 10 | 99041_at | Cluster Incl Y09973: M. musculus mRNA for TAF168/cds = (0,1760)/gb = Y09973/gi = 1842205/ug = Mm.39082/len = 1761/ | 350.3 | P | 354.8 | P | 242.2 | P | 288.7 |
| | | STRA = for | | | | | | | |
| 10 | 100929_at | Cluster Incl AW046351: UI-M-BH1-ali-a-02-0-UI.s1 Mus musculus cDNA, 3 end/clone = UI-M-BH1-ali-a-02-0-UI/ | 136.8 | P | 83.7 | P | 107.8 | P | 79 |
| | | clone_end = 3/gb AW046351/gi = 5906880/ug = Mm 6128/len = 503/STRA = for | | | | | | | |
| 10 | 102724_at | Cluster Incl AI608324: vf77b06 y1 Mus musculus cDNA, 5 end/clone = IMAGE-849779/clone_end = 5/gb = AI608324/ | 189.3 | P | 118.3 | P | 138.4 | P | 107.8 |
| | | gi = 4617491/ug = Mm 1436/len = 474/STRA = for | | | | | | | |
| 10 | 103476_at | Cluster Incl AA674794: vm74g09.s1 Mus musculus cDNA, 5 end/clone = IMAGE-1004032/clone_end = 5/gb = AA674794/ | 309 | P | 326.2 | P | 253.6 | P | 212 |
| | | gi = 2652031/ug = Mm 21926/len = 175/STRA = for | | | | | | | |
| 10 | 98306_g_at | Cluster Incl Y11245 M musculus mRNA for winged-helix transcription factor/cds = (79,2352)/gb = Y11245/gi = 2065248/ | 523.7 | P | 436.2 | P | 352.9 | P | 314 |
| | | ug = Mm 42148/len = 2673/STRA = for | | | | | | | |
| 10 | 100395_at | Cluster Incl X99104 M musculus mRNA for gli2 gene/cds = UNKNOWN/gb = X99104/gi = 1707589/ug = Mm.89929/len = 5849/ | 311.1 | P | 286.7 | P | 178.3 | P | 142.7 |
| | | STRA = for | | | | | | | |
| 10 | 92827_at | Cluster Incl AF064553 Mus musculus NSD1 protein mRNA, complete cds/cds = (1099,8865)/gb = AF064553/gi = 3329464/ | 125.2 | P | 73.3 | P | 74.6 | P | 62 |
| | | ug = Mm 12964/len = 10019/STRA = for | | | | | | | |
| 10 | 97539_at | Cluster Incl AI854416 UI-M-BH0-alt-b-02-0-UI s1 Mus musculus cDNA, 3 end/clone = UI-M-BH0-alt-b-02-0-UI/clone_end = 3/ | 72.4 | P | 81.3 | P | 64 | P | 14.9 |
| | | gb = AI854416/gi = 5498322/ug = Mm 33202/len = 504/STRA = for | | | | | | | |
| 10 | 100122_at | Cluster Incl L34290 Mus musculus guanine nucleotide regulatory protein mRNA, complete cds/cds = (30,1091)/gb = L34290/ | 207.4 | P | 244.1 | A | 203.8 | P | 210.1 |
| | | gi = 1237255/ug = Mm 4702/len = 1803/STRA = for | | | | | | | |
| 10 | 101003_at | Cluster Incl X53824 Splicing factor, arginine/serine-rich 3 (SRp20)/cds = (62,556)/gb = X53824/gi = 55439/ug = Mm 6787/ | 156.5 | P | 190.6 | P | 115.3 | P | 109.6 |
| | | len = 1239/STRA = for | | | | | | | |
| 10 | 102809_s_at | Cluster Incl M12056 Mouse rearranged Ick gene encoding lymphocte-specific protein tyrosine kinase/cds = (163,1692)/ | 174.7 | P | 97.9 | P | 114.8 | P | 149.5 |
| | | gb = M12056/gi = 198763/ug = Mm 142/len = 2100/STRA = for | | | | | | | |
| 10 | 103872_r_at | Cluster Incl AA667323: vv29b11.r1 Mus musculus cDNA, 5 end/clone = IMAGE-1223805/clone_end = 5/gb = AA667323/ | 152.9 | P | 34.1 | P | 61.1 | P | 33.1 |
| | | gi = 2626024/ug = Mm 21167/len = 210/STRA = for | | | | | | | |
| 10 | 104153_at | Cluster Incl AW047743 UI-M-BH1-alk-b-07-0-UI s1 Mus musculus cDNA, 3 end/clone = UI-M-BH1-alk-b-07-0-UI/ | 271 | P | 350.2 | P | 291.7 | P | 210.4 |
| | | clone_end = 3/gb = AW047743/gi = 5908272/ug = Mm 22199/len = 339/STRA = for | | | | | | | |
| 10 | 103516_at | Cluster Incl AF031572 Mus musculus seven-pass transmembrane receptor precursor (Celsr1) mRNA, complete cds/ | 305.1 | P | 313.6 | P | 347.2 | P | 219.3 |
| | | cds = (7,9111)/gb = AF031572/gi = 3800735/ug = Mm 22680/len = 10919/STRA = for | | | | | | | |
| 10 | 104176_at | Cluster Incl AI850941 UI-M-BH0-akd-d-10-0-UI s1 Mus musculus cDNA, 3 end/clone = UI-M-BH0-akd-d10-0-UI/ | 209.6 | P | 206.2 | P | 161.2 | P | 149.6 |
| | | clone_end = 3/gb = AI850941/gi = 5494847/ug = Mm 27261/len = 377/STRA = for | | | | | | | |
| 10 | 92914_at | Cluster Incl M18400: Mouse homeo box (Hox2 3) region/cds = (0,253)/gb = M18400/gi = 193939/ug = Mm 4822/len = 254/ | 370.2 | P | 320.3 | P | 344.4 | P | 358.8 |
| | | STRA = for | | | | | | | |
| 10 | 93212_at | Cluster Incl Z97207 Mus musculus partial mRNA, for B-IND1 protein (B-ind1 gene)/cds = (0,570)/gb = Z97207/gi = 5777593/ | 128.9 | P | 107.3 | P | 92.4 | A | 90.5 |
| | | ug = Mm.57023/len = 571/STRA = for | | | | | | | |
| 10 | 93481_at | Cluster Incl AI846720 UI-M-AN1-afi-h-09-0-UI s1 Mus musculus cDNA, 3 end/clone = UI-M-AN1-afi-b-09-0-UI/ | 582.7 | P | 386.8 | P | 606.7 | P | 323 |
| | | clone_end = 3/gb = AI846720/gi = 5490626/ug Mm 7124/len = 161/STRA = for | | | | | | | |
| 10 | 98818_at | Cluster Incl X04435: Glucocorticold receptor 1/cds = (18,2369)/gb = X04435/gi = 51057/ug = Mm 4918/len = 2575/STRA = for | 112.9 | P | 80.1 | A | 77.9 | P | 60.2 |
| 10 | 93056_g_at | Cluster Incl AW049376 UI-M-BH1-ane-c-05-0-UI s1 Mus musculus cDNA, 3 end/clone = UI-M-BH1-ane-c-05-0-UI/ | 60.3 | P | 115.7 | P | 117.4 | P | 41.3 |
| | | clone_end = 3/gb = AW049376/gi = 5909905/ug = Mm 15351/len = 492/STRA = for | | | | | | | |
| 10 | 97822_at | Cluster Incl AW122689 UI-M-BH2 2-aot-a-10-0-UI s1 Mus musculus cDNA, 3 end/clone = UI-M-BH2 2-aot-a-10-0-UI/ | 622.8 | P | 523.6 | P | 607.9 | A | 413.8 |
| | | clone_end = 3/gb = AW122689/gi = 6098319/ug = Mm 28202/len = 467/STRA = rev | | | | | | | |
| 10 | 98886_at | Cluster Incl AI835412 UI-M-AQ0-aaj-d-12-0-UI s1 Mus musculus cDNA, 3 end/clone = UI-M-AQ0-aaj-d-12-0-UI/ | 530.4 | P | 456.6 | A | 216.5 | A | 384.6 |
| | | clone_end = 3/gb = AI835412/gi = 5469625/ug = Mm 28536/len = 355/STRA = for | | | | | | | |
| 10 | 95122_g_at | Cluster Incl AW060791 UI-M-BH1-anu-f-12-0-UI s1 Mus musculus cDNA, 3 end/clone = UI-M-BH1-anu-f-12-0-UI/ | 162 | P | 113.4 | P | 93 | P | 79.6 |
| | | clone_end = 3/gb = AW060791/gi = 6008542/ug = Mm 29390/len = 519/STRA = for | | | | | | | |
| 10 | 99163_at | Cluster Incl AI844232 UI-M-AL1-ahk-f-04-0-UI.s1 Mus musculus cDNA, 3 end/clone = UI-M-AL1-ahk-f-04-0-UI/ | 199.6 | A | 188.7 | P | 277.4 | P | 329.1 |
| | | clone_end = 3/gb = AI844232/gi = 5488138/ug = Mm 41671/len = 242/STRA = for | | | | | | | |
| 10 | 94401_s_at | Cluster Incl AA986548 ue14g03 x1 Mus musculus cDNA, 3 end/clone = IMAGE-1480372/clone_end = 3/gb AA986548/ | 41.8 | A | 42.1 | P | 29.5 | P | 42.2 |
| | | gi = 3168202/ug = Mm 25793/len = 543/STRA = for | | | | | | | |
| 10 | 98003_at | Cluster Incl AF038149 Mus musculus paired immonoglobutin-like receptor B (Pirb) gene, complete cds/cds = (0,2525)/ | 352.1 | P | 322.8 | P | 269.7 | P | 423.1 |
| | | gb = AF038149/gi = 3135465/ug = Mm.3189/len = 2526/STRA = for | | | | | | | |
| 10 | 101945_g_at | Cluster Incl U89352: Mus musculus lysophospholipase I mRNA, complete cds/cds = (0,692)/gb = U89352/gi = 1864158/ | 165.7 | P | 58.9 | P | 109.4 | P | 87.9 |
| | | ug = Mm.95696/len = 693/STRA = for | | | | | | | |
| 10 | 102277_at | Cluster Incl M36514.Mouse zinc finger protein (mkr3) mRNA, end/cds = (0,1286)/gb = M36514/gi = 191507/ug = Mm 1176/ | 125.8 | P | 96.1 | P | 79.2 | P | 63.8 |
| | | len = 2470/STRA = for | | | | | | | |
| 10 | 103272_at | Cluster Incl AF091998: Mus musculus calpain Lp82 mRNA, complete cds/cds = (37,2166)/gb = AF091998/gi = 3661584/ | 448.3 | P | 515.2 | P | 548.2 | P | 374 |
| | | ug = Mm 20863/len = 2334/STRA = for | | | | | | | |
| 10 | 93410_at | Cluster Incl AW120840 UI-M-BH2 3-aoa-e-01-0-UI s1 Mus musculus cDNA, 3 end/clone = UI-M-BH2 3-aoa-e-01-0-UI/ | 245.1 | M | 348.6 | P | 216.9 | P | 234.3 |
| | | clone_end = 3/gb = AW120840/gi = 6096173/ug = Mm.6378/len = 371/STRA = for | | | | | | | |
| 10 | 100371_at | Cluster Incl U65316: Mus musculus hnRNP A1 (Hnrpa1) gene, alternative splicing region, exon 6 to partial exon 8, and | 192 | P | 149.2 | A | 134.3 | P | 154.2 |
| | | partial cds/cds = (0,381)/gb = U65316/gi = 1507693/ug Mm.88834/len = 382/STRA = for | | | | | | | |
| 10 | 92559_at | Cluster Incl U12884: Mus musculus C57BL/6 vascuar cell adhesion molecule-1 truncated form T-VCAM-1 (VCAM-1) | 28.9 | P | 45.7 | P | 24 | P | 25.1 |
| | | mRNA, complete cds/cds = (43,1080)/gb = U12884/gi = 531884/ug Mm.1021/len = 1332/STRA = for | | | | | | | |
| 10 | 92857_at | Cluster Incl AI853960 UI-M-BH0-aiv-d-02-0-UI s1 Mus musculus cDNA, 3 end/clone = UI-M-BH0-aiv-d-02-0-UI/ | 240.4 | P | 110.4 | A | 266.3 | P | 43.1 |
| | | clone_end = 3/gb = AI853960/gi = 5497866/ug = Mm.13917/len = 388/STRA = for | | | | | | | |
| 10 | 93091_s_at | Cluster Incl M63503: Mouse keratinocyte growth factor receptor mRNA, complete cds/cds = (675,2798)/gb = M63503/ | 173.8 | A | 48.4 | A | 39.6 | A | 1.6 |
| | | gi = 198593/ug Mm 16340/len 3037/STRA = for | | | | | | | |
| 10 | 97860_at | Cluster Incl AJ002198.Nucleosome assembly protein 1 like 4/cds = (66,1193)/gb = AJ002198/gi = 3319976/ug Mm 2831/ | 591.1 | P | 564.4 | P | 489.3 | P | 501.3 |
| | | len = 2301/STRA = for | | | | | | | |
| 10 | 98016_at | Cluster Incl AA981266 vx80f03 r1 Mus musculus cDNA, 5 end/clone = IMAGE-1279613/clone_end = 5/gb = AA981268/ | 210.6 | P | 174.6 | P | 144.2 | P | 90.3 |
| | | gi = 3159804/ug = Mm 3235/len = 424/STRA = for | | | | | | | |
| 10 | 102696_s_at | Cluster Incl AI747899: ul03g02.y1 Mus musculus cDNA, 5 end/clone = IMAGE-2065010/clone_end = 5/gb = AI747899/ | 204.9 | P | 65.8 | A | 161.7 | P | 124.7 |
| | | gi = 5126163/ug = Mm.14147/len = 564/STRA = rev | | | | | | | |
| 10 | 102699_at | Cluster Incl J03368: Myxovirus (influenza virus) resistance 2/cds = UNKNOWN/gb = J03368/gi = 199921/ug = Mm.14157/ | 261.3 | P | 315 | P | 217.5 | A | 243 |
| | | len = 2426/STRA = for | | | | | | | |
| 10 | 103928_at | Cluster Incl AI788018 uf16c04.y1 Mus musculus cDNA, 5 end/clone = IMAGE-2087718/clone_end = 5/gb = AI788018/ | 206.6 | P | 127.2 | P | 150.4 | A | 113.6 |
| | | gi = 5335734/ug = Mm 25096/len = 488/STRA = for | | | | | | | |
| 10 | 104170_at | Cluster Incl AF003115 Mus musculus JIP-1 (JIP-1) mRNA, complete cds/cds = (137,2119)/gb = AF03115/gi = 2316073/ | 845.8 | P | 788.4 | A | 839.8 | P | 594.9 |
| | | ug = Mm.2720/len = 2790/STRA = for | | | | | | | |
| 10 | 93400_at | Cluster Incl U88990 Apoptosis Inhibitor 3/cds = (671,2161)/gb = U88990/gi = 2138318/ug = Mm 6299/len = 2691/STRA = for | 139.6 | P | 135.6 | P | 85.6 | P | 95.7 |
| 10 | 93911_at | Cluster Incl AI315090: uj23g10 x1 Mus musculus cDNA, 3 end/clone = IMAGE-1920834/clone_end = 3/gb = AI315090/ | 258.8 | P | 84.6 | P | 133.4 | A | 155.6 |
| | | gi = 4030357/ug = Mm.89567/len = 467/STRA = for | | | | | | | |
| 10 | 101231_at | Cluster Incl C78334 C78334 Mus musculus cDNA, 3 end/clone = J0047A07/clone_end = 3/gb = C78334/gi = 2518664/ | 182.7 | P | 142.9 | A | 139.7 | P | 81.7 |
| | | ug = Mm.5703/len = 571/STRA = rev | | | | | | | |
| 10 | 102093_f_at | Cluster Incl AI461979: ub69a03 x1 Mus musculus cDNA, 3 end/clone = IMAGE-1382965/clone_end = 3/gb = AI461979/ | 78.3 | P | 93.7 | A | 65.5 | P | 68.4 |
| | | gi = 4316009/ug = Mm 42326/len = 272/STRA = rev | | | | | | | |
| 10 | 102163_at | Cluster Incl X60388 M musculus SmN pseudogene/cds = (0,725)/gb = X60388/gi = 54118/ug = Mm 46814/len = 726/ | 479.5 | P | 470.6 | P | 372 | P | 429.3 |
| | | STRA = for | | | | | | | |
| 10 | 92841_l_at | Cluster Incl X51429 Chromogranin B/cds = (52,2085)/gb = X51429/gi = 50408/ug = Mm 1339/len = 2340/STRA = for | 129.8 | P | 133.1 | P | 71.8 | P | 67 |
| 10 | 96046_at | Cluster Incl X96207 M musculus mRNA for histone deacetylase/cds = (17,1465) gb = X98207/gi = 1771285/ug = Mm 2602/ | 489.7 | P | 283 | P | 241 | P | 234.9 |
| | | len = 1977/STRA = for | | | | | | | |
| 10 | 102758_at | Cluster Incl AJ006486 Mus musculus mRNA for RNA binding protein/cds = (117,3470)/gb = AJ006486/gi = 4883436/ | 107.6 | P | 57.2 | A | 61.5 | A | 74.3 |
| | | ug = Mm 12931/len = 3593/STRA = for | | | | | | | |
| 10 | 103393_at | Cluster Incl AA738776 W67d07 r1 Mus musculus cDNA, 5 end/clone = IMAGE-1227469/clone_end = 5/gb = AA738776/ | 105.9 | P | 37.7 | A | 98.4 | M | 63.2 |
| | | gi = 2776028/ug = Mm 18659/len = 385/STRA = for | | | | | | | |
| 10 | 102631_at | Cluster Incl AB008674 Mus musculus mRNA for mBlm, complete cds/cds = (342,4592)/gb = AB008674/gi = 3370995/ | 205 | P | 48.8 | P | 137.2 | M | 121 |
| | | ug = Mm 12932/len = 4763/STRA = for | | | | | | | |
| 10 | 103200_at | Cluster Incl AA711773 vu58g05 r1 Mus musculus cDNA, 5 end/clone = IMAGE-1195640/clone_end = 5/gb = AA711773/ | 78.6 | P | 97.8 | P | 84.4 | A | 89.9 |
| | | gi = 2721691/ug = Mm 1902/len = 473/STRA = for | | | | | | | |
| 10 | 95244_at | Cluster Incl D10632 Zinc finger protein 40/cds = (222,2408)/gb = D10632/gi = 286104/ug = Mm 21025/len = 2754/STRA = for | 58.6 | P | 44.7 | A | 43.2 | P | 6.8 |
| 10 | 101677_at | Cluster Incl AI450336 mu27g11 x1 Mus musculus cDNA, 3 end/clone = IMAGE-640676/clone_end = 3/gb = AI450336/ | 251 | P | 116.2 | A | 113 | A | 136.2 |
| | | gi = 4296290/ug = Mm 32307/len = 451/STRA = rev | | | | | | | |
| 11 | 92553_at | Cluster Incl AB025408 Mus musculus mRNA for sid478p, complete cds/cds = (74,922)/gb = AB025408/gi = 5931564/ | 1128.9 | P | 1065 | P | 1007 | P | 861.8 |
| | | ug = Mm.1017/len = 1056/STRA = for | | | | | | | |
| 11 | 92589_at | Cluster Incl AI846545 UI-M-AN1-aff-g-12-0-UI s1 Mus musculus cDNA, 3 end/clone = UI-M-AN1-aff-g-12-0-UI/ | 1033 | P | 1003 | P | 856.1 | P | 785.2 |
| | | clone_end = 3/gb = AI846545/gi = 5490451/ug = Mm 10623/len = 354/STRA = for | | | | | | | |
| 11 | 92595_r_at | Cluster Incl M61215 Mus musculus ferrochelatase mRNA, complete cds/cds = (92,1354)/gb = M61215/gi = 19362/ | 136.7 | P | 174.2 | P | 125.4 | P | 84.4 |
| | | ug = Mm.1070/len = 2897/STRA = for | | | | | | | |
| 11 | 92608_at | Cluster Incl D88793 Cysteine rich protein/cds = (130,711)/gb = D88793/gi = 1695703/ug = Mm 10919/len = 938/STRA = for | 1151.1 | P | 1113.5 | P | 1289.9 | P | 1045.2 |
| 11 | 92632_at | Cluster Incl AI842328: UI-M-AM1-afz-b-11-0-UI s1 Mus musculus cDNA, 3 end/clone = UI-M-AM1-afz-b-11-0-UI/ | 1002.9 | P | 1141 | P | 1135.5 | P | 904.2 |
| | | clone_end = 3/gb = AI842328/gi = 5476506/ug = Mm 1147/len = 389/STRA = for | | | | | | | |
| 11 | 92636_f_at | Cluster Incl U11027 Mus musculus C57BL/6J sec61 protein complex gamma subunit mRNA, complete cds/cds = (3,209)/ | 2098.9 | P | 2628 | P | 1941.7 | P | 2009.9 |
| | | gb = U11027/gi = 506791/ug = Mm.1164/len = 367/STRA = for | | | | | | | |
| 11 | 92646_at | Cluster Incl U84903 Ribosomal protein L23/cds = (61,501)/gb = U84903/gi = 2565133/ug = Mm 12144/len = 582/STRA = for | 996.4 | P | 1328.8 | P | 1174.8 | P | 857.3 |
| 11 | 92798_at | Cluster Incl AA870675 vq24f11.r1 Mus musculus cDNA, 5 end/clone = IMAGE-1095213/clone_end = 5/gb = AA870675/ | 3036.7 | P | 3137.6 | P | 2778.5 | P | 2655.1 |
| | | gi = 2966120/ug = Mm.12677/len = 405/STRA = for | | | | | | | |
| 11 | 92799_g_at | Cluster Incl AA870675 vq24f11.r1 Mus musculus cDNA, 5 end/clone = IMAGE-1095213/clone_end = 5/gb = AA870675/ | 2666.5 | P | 2973 | P | 2887.4 | P | 2732.8 |
| | | gi = 2966120/ug = Mm 12677/len = 405/STRA = for | | | | | | | |
| 11 | 92810_at | Cluster Incl AI842259 UI-M-AI1-afp-f-09-0-UI s1 Mus musculus cDNA, 3 end/clone = UI-M-AI1 -afp-f-09-0-UI/clone_end = 3/ | 501.8 | P | 482.8 | P | 570.4 | P | 353.1 |
| | | gb = AI842259/gi = 5476472/ug = Mm.12775/len = 389/STRA = for | | | | | | | |
| 11 | 92845_at | Cluster Incl AI843232 UI-M-AK1-aet-h-03-0-UI s1 Mus musculus cDNA, 3 end/clone = UI-M-AK1-aet-h-03-0-UI/ | 600.2 | P | 571 | P | 633.2 | P | 240 |
| | | clone_end = 3/gb = AI843232/gi 5477445/ug = Mm 13445/len = 396/STRA = for | | | | | | | |
| 11 | 93057_at | Cluster Incl AW122989 UI-M-BH2.1-apb-b-08-0-UI s1 Mus musculus cDNA, 3 end/clone = UI-M-BH2 1-apb-b-08-0-UI/ | 3053.4 | P | 3421.1 | P | 3326.3 | P | 3323 |
| | | clone_end = 3/gb = AW122989/gi = 6098519/ug = Mm 1538/len = 479/STRA = for | | | | | | | |
| 11 | 93094_at | Cluster Incl U88588 Cerebellar degeneration-related 2/cds = (153,1520)/gb = U88588/gi = 1857920/ug = Mm 1640/len = 2432/ | 258.1 | P | 272.2 | P | 253.6 | P | 146.7 |
| | | STRA = for | | | | | | | |
| 11 | 93102_f_at | Cluster Incl U20365: Actin, gamma 2, smooth muscle, enteric/cds = (36,1217)/gb = U20365/gi = 950001/ug = Mm 16562/ | 1294.9 | P | 1798.6 | P | 1780.1 | P | 764.7 |
| | | len = 1232/STRA = for | | | | | | | |
| 11 | 93119_at | Cluster Incl X53157 Mouse mRNA for mitochondrial cytochrome c oxidase subunit Vb/cds = (12,398)/gb = X53157/gi = 50530/ | 2660.2 | P | 3316.9 | P | 2731.9 | P | 2886.8 |
| | | ug = Mm 16769/len = 475/STRA = for | | | | | | | |
| 11 | 93126_at | Cluster Incl X04591 Mouse mRNA 3 region for brain creatine kinase B/cds = (0,86) gb = X04591/gi = 50413/ug = Mm 16831/ | 986 | P | 1116.1 | P | 1197.7 | P | 537.7 |
| | | len = 285/STRA = for | | | | | | | |
| 11 | 93276_at | Cluster Incl U90123.Hematological and neurological and neurological expressed sequence 1/cds = (95,559) gb = U90123/gi = | 1304.8 | P | 1436.4 | P | 1465.8 | P | 1085 |
| | | 1864164/ug = Mm 1775/len = 1384/STRA = for | | | | | | | |
| 11 | 93356_at | Cluster Incl D26091 Mus musculus mRNA for mCDC47, complete cds/cds = (106,2265)/gb = D26091/gi = 1136746/ | 518 | P | 585.7 | P | 508.9 | P | 341.3 |
| | | ug = Mm.18923/len = 2396/STRA = for | | | | | | | |
| 11 | 93495_at | Cluster Incl U96746 Mus musculus antioxidant enzyme AOE372 mRNA, complete cds/cds = (90,914) gb = U96746/ | 635.8 | P | 897.8 | P | 1016.5 | P | 525.9 |
| | | gi = 2104954/ug = Mm 19127/len = 977/STRA = for | | | | | | | |
| 11 | 93539_at | Cluster Incl AI118854: uc14105.r1 Mus musculus cDNA, 5 end/clone = IMAGE-1397985/clone_end = 5/gb = AI118854/ | 556 | P | 700.5 | P | 662.2 | P | 387.7 |
| | | gi = 3519178/ug = Mm.2000/len = 281/STRA = for | | | | | | | |
| 11 | 93575_at | Cluster Incl AF051102: Mus musculus gamma glutamyl hydrolase precursor, mRNA, complete cds/cds = (90,1043)/ | 622.3 | P | 553.2 | P | 816.4 | P | 289.8 |
| | | gb = AF051102/gi = 3831561/ug = Mm 20461/len = 1295/STRA = for | | | | | | | |
| 11 | 93812_at | Cluster Incl U53455 Mus musculus chloride ion current inducer protein (GLCI) mRNA, partial cds/cds = (0,710)/gb = U53455/ | 829.3 | P | 829.4 | P | 805.6 | P | 421 |
| | | gi = 1399647/ug = Mm.21482/len = 711/STRA = for | | | | | | | |
| 11 | 93830_at | Cluster Incl AI851199 UI-M-BH0-ajx-e-10-0-UI s1 Mus musculus cDNA, 3 end/clone = UI-M-BH0-ajx-e-10-0-UI/ | 2989.9 | P | 2820.6 | P | 2976.6 | P | 2449.3 |
| | | clone_end = 3/gb = AI851199/gi = 5495105/ug = Mm.21559/len = 301/STRA = for | | | | | | | |
| 11 | 93839_at | Cluster Incl AI854888 UI-M-BH0-akb-g-05-0-UI.s1 Mus musculus cDNA, 3 end/clone = UI-M-BH0-akb-g-05-0-UI/ | 1060.1 | P | 1014.2 | P | 1041.2 | P | 750.4 |
| | | clone_end = 3/gb = AI854888/gi = 5498794/ug = Mm 21606/len = 468/STRA = for | | | | | | | |
| 11 | 93984_at | Cluster Incl AF002718 Mus musculus ATPase inhibitor (IF1) mRNA, complete cds/cds = (30,350)/gb = AF002718/ | 2698.2 | P | 2698.1 | P | 2601.2 | P | 2345.1 |
| | | gi = 2367440/ug = Mm 2171/len = 509/STRA = for | | | | | | | |
| 11 | 94032_at | Cluster Incl AI845103 UI-M-BG0-ahs-a-01-0-UI s1 Mus musculus cDNA, 3 end/clone = UI-M-BG0-ahs-a-01-0-UI/ | 1110 | P | 1091.2 | P | 1256.6 | P | 811.2 |
| | | clone_end = 3/gb = AI845103/gi = 5489009/ug = Mm 21905/len = 309/STRA = for | | | | | | | |
| 11 | 94038_at | Cluster Incl AA675468: vr72c04 s1 Mus musculus cDNA, 5 end/clone = IMAGE-1134246/clone_end = 5/gb = AA675468/ | 1279.9 | P | 1518.5 | P | 1400.5 | P | 1204.3 |
| | | gi = 2652705/ug = Mm.21932/len = 540/STRA = for | | | | | | | |
| 11 | 94047_at | Cluster Incl AW122935 UI-M-BH2.1-apa-h-12-0-UI s1 Mus musculus cDNA, 3 end/clone = UI-M-BH2.1-apa-h-12-0-UI/ | 617.8 | P | 658.9 | P | 815 | P | 637.1 |
| | | clone_end = 3/gb = AW122935/gi = 6098465/ug = Mm 21976/len = 365/STRA = for | | | | | | | |
| 11 | 94206_at | Cluster Incl AC002397 Mouse chromosome 6 Bac-284H12 (Research Genetics mouse BAC library) complete sequence/ | 1035.7 | P | 1082.5 | P | 1198.5 | P | 932 |
| | | cds = (108,488)/gb = AC002397/gi = 3287367/ug = Mm 22195/len = 568/STRA = for | | | | | | | |
| 11 | 94210_at | Cluster Incl AW121333 UI-M-BH2.2-aom-b-04-0-UI.s1 Mus musculus cDNA, 3 end/clone = UI-M-BH2.2-aom-b-04-0-UI/ | 1093.4 | P | 1214 | P | 1248.5 | P | 1159.3 |
| | | clone_end = 3/gb = AW121333/gi = 6096666/ug = Mm 22201/len = 494/STRA = for | | | | | | | |
| 11 | 94229_at | Cluster Incl AW124489: UI-M-BH2.1-apo-a-01-0-UI s1 Mus musculus cDNA, 3 end/clone = UI-M-BH2.1-apo-a-01-0-UI/ | 566.7 | P | 580.9 | P | 705.5 | P | 491.9 |
| | | clone_end = 3/gb = AW124489/gi = 6099984/ug = Mm 22281/len = 513/STRA = for | | | | | | | |
| 11 | 94252_at | Cluster Incl AJ006587 Mus musculus mRNA for translation initiation factor eIF2 gamma X/cds = (8,1426)/gb = AJ006587/ | 3027.6 | P | 3000.5 | P | 2970.9 | P | 2068 |
| | | gi = 3790183/ug = Mm 22387/len = 1787/STRA = for | | | | | | | |
| 11 | 94266_at | Cluster Incl AW121737: UI-M-BH2-3-aoe-b-10-0-UI s1 Mus musculus cDNA, 3 end/clone = UI-M-BH2-3-aoe-b-10-0-UI/ | 1181 | P | 1659.7 | P | 1948.2 | P | 1016.2 |
| | | clone_end = 3/gb = AW121737/gi = 6097070/ug = Mm 22469/len = 365/STRA = for | | | | | | | |
| 11 | 94811_s_at | Cluster Incl AJ002366 Mus musculus mRNA for transcription factor TFIIH, 62 kD subunit/cds = (44,1687)/gb = AJ002266/ | 213.5 | P | 356.4 | P | 437.4 | P | 144.7 |
| | | gi = 2582796/ug = Mm 22700/len = 2471/STRA = for | | | | | | | |
| 11 | 94817_at | Cluster Incl X60676 M musculus HSP47 mRNA/cds = (256,1509)/gb = X60676/gi = 51449/ug = Mm 22708/len = 2273/ | 3203.9 | P | 3521.1 | P | 4107.3 | P | 2566.5 |
| | | STRA = for | | | | | | | |
| 11 | 94820_r_at | Cluster Incl AF0058886 Mus musculus cyclin I mRNA, complete cds/cds = (286,1419)/gb = AF005886/gi = 4101686/ | 837.4 | P | 885 | P | 1334.7 | P | 993.9 |
| | | ug = Mm 22711/len = 1548/STRA = for | | | | | | | |
| 11 | 94915_at | Cluster Incl X58990 Mouse mCyp-S1 mRNA for cyclophilin CyP-S1/cds = (166,792)/gb = X58990/gi = 53034/ug = Mm 2412/ | 2001.9 | P | 2396.1 | P | 2283.6 | P | 1911.2 |
| | | len = 998/STRA = for | | | | | | | |
| 11 | 95430_f_at | Cluster Incl AI854154 UI-M-BH0-ajw-h-03-0-UI.s1 Mus musculus cDNA, 3 end/clone = UI-M-BH0-ajw-h-03-0-UI/ | 546.5 | P | 899.7 | P | 1020.5 | P | 836.3 |
| | | clone_end = 3/gb = AI854154/gi = 5498060/ug = Mm 24460/len = 561/STRA = for | | | | | | | |
| 11 | 95493_at | Cluster Incl X66405 Procollagen, type VI, alpha 1/cds = (131,3208)/gb = X66405/gi = 50478/ug = Mm 2509/len = 3991/ | 2809 | P | 3585.9 | P | 3648.6 | P | 2981.2 |
| | | STRA = for | | | | | | | |
| 11 | 96057_at | Cluster Incl AI647493 uk42h02.x1 Mus musculus cDNA, 3 end/clone = IMAGE-1971699/clone_end = 3/gb = AI647493/ | 617.8 | P | 670.2 | P | 714.5 | P | 552.5 |
| | | gi = 4726171/ug = Mm 2621/len = 761/STRA = for | | | | | | | |
| 11 | 96058_s_at | Cluster Incl U07235 Mus musculus aldehyde dehydrogenase (ALDH2) mRNA, nuclear gene encoding mitochondrial | 432 | P | 465.1 | P | 629.1 | P | 334.3 |
| | | protein, complete cds/cds = (105,1664)/gb = U07235/gi = 466253/ug = Mm 2621/len = 1954/STRA = for | | | | | | | |
| 11 | 96059_at | Cluster Incl AA823336 vp35g10 r1 Mus musculus cDNA, 5 end/clone = IMAGE-1078722/clone_end = 5/gb = AA823336/ | 482.1 | P | 486.9 | P | 450.3 | P | 369.4 |
| | | gi = 2893204/ug = Mm 26217/len = 366/STRA = for | | | | | | | |
| 11 | 96086_at | Cluster Incl AW122530 UI-M-BH2.2-aox-b-12-0-UI s1 Mus musculus cDNA, 3 end/clone = UI-M-BH2.2-aox-b-12-0-UI/ | 459.7 | P | 397.3 | P | 488.2 | P | 372.2 |
| | | clone_end = 3/gb = AW122530/gi = 6098054/ug = Mm 26680/len = 206/STRA = for | | | | | | | |
| 11 | 96115_at | Cluster Incl U28168: Adenomatosis polyposis coli/cds = (35,592)/gb = U28168/gi = 1142715/ug = Mm 2698/len = 719/ | 2463.4 | P | 2820.9 | P | 2497.9 | P | 2368.6 |
| | | STRA = for | | | | | | | |
| 11 | 96617_at | Cluster Incl AI844737 UI-M-AL1-ahq-c-11-0-UI s1 Mus musculus cDNA, 3 end/clone = UI-M-AL1-ahq-c-11-0-UI/ | 654.5 | P | 675.9 | P | 564.9 | P | 523.9 |
| | | clone_end = 3/gb = AI844737/gi = 5488643/ug = Mm 27128/len = 178/STRA = for | | | | | | | |
| 11 | 96652_at | Cluster Incl AI849911: UI-M-BG0-ahz-d-03-0-UI s1 Mus musculus cDNA, 3 end/clone = UI-M-BG0-ahz-d-03-0-UI/ | 572.5 | P | 628.1 | P | 574.2 | P | 409.7 |
| | | clone_end = 3/gb = AI849911/gi = 5493817/ug = Mm 27332/len = 385/STRA = for | | | | | | | |
| 11 | 96661_at | Cluster Incl AA734806 vp38b03 r1 Mus musculus cDNA, 5 end/clone = IMAGE-1078925/clone_end = 5/gb = AA734806/ | 1089.3 | P | 1288.1 | P | 1281.5 | P | 752 |
| | | gi = 2756473/ug = Mm.27357/len = 446/STRA = for | | | | | | | |
| 11 | 96667_at | Cluster Incl AI851542 UI-M-BH0-aki-d-01-0-UI.s1 Mus musculus cDNA, 3 end/clone = UI-M-BH0-aki-d-01-0-UI/ | 233.6 | P | 269.1 | P | 466.1 | P | 263.1 |
| | | clone_end = 3/gb = AI851542/gi = 5495448/ug = Mm.27389/len = 477/STRA = for | | | | | | | |
| 11 | 96668_at | Cluster Incl AI845867 UI-M-AO1-aeg-g-01-0-UI s1 Mus musculus cDNA, 3 end/clone = UI-M-AO1-aeg-g-01-0-UI/ | 1014.3 | P | 973.1 | P | 1029.3 | P | 890.5 |
| | | clone_end = 3/gb = AI845867/gi = 5489773/ug = Mm 27393/len = 345/STRA = for | | | | | | | |
| 11 | 98710_at | Cluster Incl AI854262 UI-M-BH0-aja-b-11-0-UI s1 Mus musculus cDNA, 3 end/clone = UI-M-BH0-aja-b-11-0-UI/ | 1373.7 | P | 1689.7 | P | 1944.6 | P | 974.1 |
| | | clone_end = 3/gb = AI854262/gi = 5498168/ug = Mm 27624/len = 547/STRA = for | | | | | | | |
| 11 | 97268_i_at | Cluster Incl AI848699 UI-M-AH1-agv-g-05-0-UI.s1 Mus musculus cDNA, 3 end/clone = UI-M-AH1-agv-g-05-0-UI/ | 1093.8 | P | 1026.8 | P | 1165.6 | P | 980.1 |
| | | clone_end = 3/gb = AI848699/gi = 5492600/ug = Mm 27904/len = 531/STRA = for | | | | | | | |
| 11 | 97269_f_at | Cluster Incl AI848699.UI-M-AH1-agv-g-05-0-UI.s1 Mus musculus cDNA, 3 end/clone = UI-M-AH1-agv-g-05-0-UI/ | 1891.1 | P | 1363 | P | 2776.3 | P | 1608.8 |
| | | clone_end = 3/gb = AI848699/gi = 5492600/ug = Mm.27904/len = 531/STRA = for | | | | | | | |
| 11 | 97797_at | Cluster Incl AW123142.UI-M-BH2.1-apd-b-05-0-UI s1 Mus musculus cDNA, 3 end/clone = UI-M-BH2.1-apd-b-05-0-UI/ | 841.7 | P | 753.7 | P | 856.5 | P | 704.5 |
| | | clone_end = 3/gb = AW123142/gi = 6098672/ug = Mm 28111/len = 572/STRA = for | | | | | | | |
| 11 | 97820_at | Cluster Incl AB027012 Mus musculus galK mRNA for galactokinase, complete cds/cds = (0,1175)/gb = AB027012/ | 305.5 | P | 452.5 | P | 326.2 | P | 190.5 |
| | | gi = 5931607/ug = Mm.2820/len = 1293/STRA = for | | | | | | | |
| 11 | 97828_at | Cluster Incl AF033115 Mus musculus proapoptotic protein (Siva) gene, complete cds/cds = (0,527)/gb = AF0033115/ | 687.1 | P | 676.6 | P | 550.1 | P | 343 |
| | | gi = 2921301/ug = Mm 28216/len = 528/STRA = for | | | | | | | |
| 11 | 97863_at | Cluster Incl AW125274: UI-M-BH2.1-aps-h-08-0-UI.s1 Mus musculus cDNA, 3 end/clone = UI-M-BH2.1-aps-h-08-0-UI/ | 381.6 | P | 482.8 | P | 527.4 | P | 395.3 |
| | | clone_end = 3/gb = AW125274/gi = 6100804/ug = Mm.28326/len = 148/STRA = for | | | | | | | |
| 11 | 97866_at | Cluster Incl AI842858: UI-M-AN1-1-afb-c-09-0-UI.s1 Mus musculus cDNA, 3 end/clone = UI-M-AN1-afb-c-09-0-UI/ | 467.8 | P | 398.3 | P | 475.9 | P | 346.3 |
| | | clone_end = 3/gb = AI842858/gi = 5477071/ug = Mm 28327/len = 254/STRA = for | | | | | | | |
| 11 | 97909_at | Cluster Incl AI838080 UI-M-AL0-abv-e-12-0-UI.s1 Mus musculus cDNA, 3 end/clone = UI-M-AL0-abv-e-12-0-UI/ | 3263 | P | 3621.5 | P | 2968.5 | P | 1317 |
| | | clone_end = 3/gb = AI838080/gi = 5472293/ug = Mm.28479/len = 226/STRA = for | | | | | | | |
| 11 | 98953_at | Cluster Incl AW048032 UI-M-BH1-alv-b-08-0-UI s1 Mus musculus cDNA, 3 end/clone = UI-M-BH1-alv-b-08-0-UI/ | 601.9 | P | 597 | P | 559.7 | P | 513.1 |
| | | clone_end = 3/gb = AW048032/gi = 5908526/ug = Mm.28753/len = 587/STRA = for | | | | | | | |
| 11 | 94499_at | Cluster Incl AI835427 UI-M-AQ0-aaj-f-10-0-UI s1 Mus musculus cDNA, 3 end/clone = UI-M-AQ0-aaj-f-10-0-UI/ | 435.8 | P | 392.7 | P | 684.1 | P | 291.3 |
| | | clone_end = 3/gb = AI835427/gi = 5469640/ug = Mm 28938/len = 236/STRA = for | | | | | | | |
| 11 | 94552_at | Cluster Incl AI839477 UI-M-AL0-abu-e-11-0-UI s2 Mus musculus cDNA, 3 end/clone = UI-M-AL0-abu-e-11-0-UI/ | 3228.8 | P | 3224.4 | P | 3558 | P | 3246.2 |
| | | clone_end = 3/gb = AI839477/gi = 54773690/ug = Mm 29089/len = 474/STRA = for | | | | | | | |
| 11 | 94558_g_at | Cluster Incl AI850893 UI-M-BH0-ajt-g-07-0-UI s1 Mus musculus cDNA, 3 end/clone = UI-M-BH0-ajt-g-07-0-UI/clone_end = 3/ | 519.4 | P | 626.3 | P | 593.8 | P | 414.8 |
| | | gb = AI850893/gi = 5494799/ug = Mm.29105/len = 375/STRA = for | | | | | | | |
| 11 | 95053_s_at | Cluster Incl AA674669 vq02d04.r1 Mus musculus cDNA, 5 end/clone = IMAGE-1093063/clone_end = 5/gb = AA674669/ | 1947.7 | P | 2170.2 | P | 1795.8 | P | 1626.4 |
| | | gi = 2651906/ug = Mm 29141/len = 414/STRA = for | | | | | | | |
| 11 | 95076_at | Cluster Incl AI848476 UI-M-AP1-agf-a-07-0-UI s2 Mus musculus cDNA, 3 end/clone = UI-M-AP1-agf-a-07-0-UI/ | 1706.9 | P | 1579.8 | P | 1819.4 | P | 1410.6 |
| | | clone_end = 3/gb = AI848476/gi = 5492382/ug = Mm.29202/len = 304/STRA = for | | | | | | | |
| 11 | 95090_at | Cluster Incl AA688600 vu18f09 r1 Mus musculus cDNA, 5 end/clone = IMAGE-1181033/clone_end = 5/gb = AA688600/ | 1268.8 | P | 1307.9 | P | 1213.6 | P | 1026.4 |
| | | gi = 2678029/ug = Mm 29269/len = 362/STRA = rev | | | | | | | |
| 11 | 95100_at | Cluster Incl AI847314 UI-M-AN1-afh-e-04-0-UI s1 Mus musculus cDNA, 3 end/clone = UI-M-AN1-afh-e-04-0-UI/ | 2600.8 | P | 2838.4 | P | 2469.6 | P | 2371.3 |
| | | clone_end = 3/gb = AI847314/gi = 5491220/ug = Mm 29341/len = 379/STRA = for | | | | | | | |
| 11 | 95120_at | Cluster Incl AI837621. UI-M-AK0-adg-d-09-0-UI.s1 Mus musculus cDNA, 3 end/clone = UI-M-AK0-adg-d-09-0-UI/ | 193 | P | 211.5 | P | 211 | P | 160.7 |
| | | clone_end = 3/gb = AI837621/gi = 5471834/ug = Mm 29386/len = 473/STRA = for | | | | | | | |
| 11 | 95652_at | Cluster Incl AF110520 Mus musculus major histocompatibility complex region NG27, NG28, RPS28, NADH | 2509.8 | P | 2834.4 | P | 3061.6 | P | 2535.1 |
| | | oxidoreductase, NG29, KIFC1, Fas-binding protein, BING1, tapasin, RalGDS-like, KE2, BING4, beta 1,3-galactosyl | | | | | | | |
| | | transferase, and RPS18 genes, complete cds, S/STRA = for | | | | | | | |
| 11 | 95662_at | Cluster Incl AW211667 uo83c04 y1 Mus musculus cDNA, 5 end/clone = IMAGE-2649126/clone_end = 5/gb = AW211667/ | 240.8 | P | 268.7 | P | 249.9 | P | 189.6 |
| | | gi = 6517563/ug = Mm 29562/len = 454/STRA = for | | | | | | | |
| 11 | 95674_r_at | Cluster Incl AI851985 UI-M-BH0-aiq-d-08-0-UI s1 Mus musculus cDNA, 3 end/clone = UI-M-BH0-aiq-d-08-0-UI/ | 1657.6 | P | 1830 | P | 2077.9 | P | 1553.7 |
| | | clone_end = 3/gb = AI851985/gi = 5495891/ug = Mm 29586/len = 404/STRA = for | | | | | | | |
| 11 | 95689_at | Cluster Incl AI840995 UI-M-AK0-adk-g-06-0-UI s1 Mus musculus cDNA, 3 end/clone = UI-M-AK0-adk-g-06-0-UI/ | 799 | P | 1149 | P | 1224.4 | P | 745.3 |
| | | clone_end = 3/gb = AI840995/gi = 5475208/ug = Mm 29650/len = 438/STRA = for | | | | | | | |
| 11 | 95715_at | Cluster Incl AW125739 UI-M-BH2 2-aqi-g-05-0-UI s1 Mus musculus cDNA, 3 end/clone = UI-M-BH2-agi-g-05-0-UI/ | 2318.6 | P | 2268.9 | P | 3230.1 | P | 2297.9 |
| | | clone_end = 3/gb = AW125739/gi = 6101234/ug = Mm 29714/len = 321/STRA = for | | | | | | | |
| 11 | 96272_at | Cluster Incl AI848471 UI-M-AH1-ags-h-12-0-UI s1 Mus musculus cDNA, 3 end/clone = UI-M-AH1-ags-h-12-0-UI/ | 756.7 | P | 739.3 | P | 932.5 | P | 665.4 |
| | | clone_end = 3/gb = AI848471/gi = 5492377/ug = Mm 29855/len = 427/STRA = for | | | | | | | |
| 11 | 96284_at | Cluster Incl AW121446 UI-M-BH2.2-aon-d-06-0-UI s1 Mus musculus cDNA, 3 end/clone = UI-M-BH2 2-aon-d-06-0-UI/ | 850.6 | P | 945.2 | P | 1015.7 | P | 805.8 |
| | | clone_end = 3/gb = AW121446/gi = 6096779/ug = Mm 29873/len = 220/STRA = for | | | | | | | |
| 11 | 96285_at | Cluster Incl AJ001616 Mus musculus mRNA for myeloid associated differentiation protein/cds = (187,1077)/gb = AJ001616/ | 1631.7 | P | 2000.6 | P | 2339.7 | P | 1485.5 |
| | | gi = 2463264/ug = Mm 29874/len = 2822/STRA = for | | | | | | | |
| 11 | 96320_at | Cluster Incl AW121862 UI-M-BH2.3-aoi-c-03-0-UI.s1 Mus musculus cDNA, 3 end/clone = UI-M-BH2-aoi-c-03-0-UI/ | 606.3 | P | 649.8 | P | 711.5 | P | 585 |
| | | clone_end = 3/gb = AW121862/gi = 6097236/ug = Mm 29937/len = 495/STRA = for | | | | | | | |
| 11 | 96345_at | Cluster Incl AI837012 UI-M-AJ0-aaz-h-08-0-UI s1 Mus musculus cDNA, 3 end/clone = UI-M-AJ0-aaz-h-08-0-UI/ | 599.8 | P | 692.9 | P | 652.4 | P | 494.2 |
| | | clone_end = 3/gb = AI837012/gi = 5471225/ug = Mm 29995/len = 460/STRA = for | | | | | | | |
| 11 | 96346_at | Cluster Incl AI854020 UI-M-BH0-ajm-b-01-0-UI s1 Mus musculus cDNA, 3 end/clone = UI-M-BH0-ajm-b-01-0-UI/ | 1049.8 | P | 1619.1 | P | 1836.1 | P | 828.5 |
| | | clone_end = 3/gb = AI854020/gi = 5497926/ug = Mm 29996/len = 262/STRA = for | | | | | | | |
| 11 | 96869_at | Cluster Incl AW124839 UI-M-BH2.1-apk-f-02-0-UI s1 Mus musculus cDNA, 3 end/clone = UI-M-BH2 1-apk-f-02-0-UI/ | 2761 | P | 3113.4 | P | 3441.6 | P | 2856.4 |
| | | clone_end = 3/gb = AW124839/gi = 6100369/ug = Mm 30064/len = 465/STRA = for | | | | | | | |
| 11 | 96887_at | Cluster Incl Y08702 M musculus mRNA for neuronal protein 15.6/cds = (51,452)/gb = Y08702/gi = 1771305/Mm.30084/ | 1634.2 | P | 2086.7 | P | 2023.6 | P | 1782 |
| | | len = 554/STRA = for | | | | | | | |
| 11 | 96888_at | Cluster Incl AI839814 UI-M-AN0-acq-b-07-0-UI s1 Mus musculus cDNA, 3 end/clone = UI-M-AN0-acq-b-07-0-UI/ | 3163.8 | P | 3795.6 | P | 3572.7 | P | 3286.9 |
| | | clone_end = 3/gb = AI839814/gi = 5473992/ug = Mm 30085/len = 342/STRA = for | | | | | | | |
| 11 | 96890_at | Cluster Incl AW060971 UI-M-BH1-anv-h-09-0-UI s1 Mus musculus cDNA, 3 end/clone = UI-M-BH1-anv-h-09-0-UI/ | 368.5 | P | 358.9 | P | 395.5 | P | 222.4 |
| | | clone_end = 3/gb = AW060971/gi = 6008722/ug = Mm 30092/len = 539/STRA = for | | | | | | | |
| 11 | 96911_at | Cluster Incl U34960 Mouse G protein beta 2 subunit mRNA, complete cds/cds = (0,1022)/gb = U34960/gi = 984550/ | 2094.9 | P | 2310.2 | P | 2175.7 | P | 1834.3 |
| | | ug = Mm 30141/len = 1023/STRA = for | | | | | | | |
| 11 | 96915_f_at | Cluster Incl AA879764 vv98d04 r1 Mus musculus cDNA, 5 end/clone = IMAGE-1230439/clone_end = 5/gb = AA879764/ | 1480.6 | P | 1840.1 | P | 2178 | P | 1648.6 |
| | | gi = 2988747/ug = Mm 30150/len = 357/STRA = for | | | | | | | |
| 11 | 96948_at | Cluster Incl AI845337 UI-M-BG0-ahx-b-12-0-UI.s1 Mus musculus cDNA, 3 end/clone = UI-M-BG0-ahx-b-12-0-UI/ | 711.4 | P | 852.2 | P | 824.4 | P | 640.3 |
| | | clone_end = 3/gb = AI845337/gi = 5489243/ug = Mm 30204/len = 210/STRA = for | | | | | | | |
| 11 | 97446_at | Cluster Incl AW125010 UI-M-BH2.1-apv-g-03-0-UI s1 Mus musculus cDNA, 3 end/clone = UI-M-BH2 1-apv-g-03-0-UI/ | 272.6 | P | 288.7 | P | 346.2 | P | 266.1 |
| | | clone_end = 3/gb = AW125010/gi = 6100540/ug = Mm 30243/len = 428/STRA = for | | | | | | | |
| 11 | 97452_at | Cluster Incl AI853078 UI-M-BH0-ajf-a-06-0-UI.s1 Mus musculus cDNA, 3 end/clone = UI-M-BH0-ajf-a-06-0-UI/ | 706.3 | P | 801.5 | P | 918.8 | P | 792.3 |
| | | clone_end = 3/gb = AI853078/gi = 5496984/ug = Mm 30255/len = 429/STRA = rev | | | | | | | |
| 11 | 97464_at | Cluster Incl AI844416 UI-M-AL1-ahp-g-01-0-UI s1 Mus musculus cDNA, 3 end/clone = UI-M-AL1-ahp-g-01-0-UI/ | 1131.9 | P | 1167.6 | P | 1350.8 | P | 851 |
| | | clone_end = 3/gb = AI844416/gi = 5488322/ug = Mm 3035/len = 432/STRA = for | | | | | | | |
| 11 | 98120_at | Cluster Incl AI844807 UI-M-AH1-agx-b-03-0-UI s1 Mus musculus cDNA, 3 end/clone = UI-M-AH1-agx-b-03-0-UI/ | 674 | P | 537.6 | P | 577.3 | P | 459.3 |
| | | clone_end = 3/gb = AI844807/gi = 5488713/ug = Mm 34951/len = 517/STRA = for | | | | | | | |
| 11 | 98147_at | Cluster Incl AC002397: Mouse chromosome 6 BAC-284H12 (Research Genetics mouse BAC library) complete sequence/ | 888.7 | P | 898.4 | P | 782.6 | P | 832.7 |
| | | cds = (52,2628)/gb = AC002397/gi = 3287367/ug = Mm 3571/len = 3176/STRA = rev | | | | | | | |
| 11 | 98149_s_at | Cluster Incl AW046496: UI-M-BH1-akz-d-01-0-UI.s1 Mus musculus cDNA, 3 end/clone = UI-M-BH1-akz-d-01-0-UI/ | 381 | P | 445.7 | P | 490.5 | P | 390.8 |
| | | clone _end = 3/gb = AW046496/gi = 5907025/ug = Mm.3572/len = 413/STRA = for | | | | | | | |
| 11 | 98516_at | Cluster Incl AI853136: UI-M-BH0-ajf-f-11-0-UI s1 Mus musculus cDNA, 3 end/clone = UI-M-BH0-ajf-f-11-0-UI/clone_end = 3/ | 959.4 | P | 903.8 | P | 980.5 | P | 884 |
| | | gb = AI853136/gi = 5497042/ug = Mm.35790/len = 291/STRA = for | | | | | | | |
| 11 | 98535_at | Cluster Incl AF076156 Calechol-O-methyltransferase/cds = (163,960)/gb = AF076156/gi = 3493252/ug = Mm 35853/ | 648 | P | 836.5 | P | 1170.7 | P | 787.4 |
| | | len = 1255/STRA = for | | | | | | | |
| 11 | 98544_at | Cluster Incl U53514 Mus musculus guanylate kinase (gmk) mRNA, complete cds/cds = (24,620)/gb = U53514/gi = 1408589/ | 629.7 | P | 638.1 | P | 503.5 | P | 437.4 |
| | | ug = Mm 3624/len = 663/STRA = for | | | | | | | |
| 11 | 98627_at | Cluster Incl X81580: Insulin-like growth factor binding protein 2/cds = (71,988)/gb = X81580 gi = 550378/ug = Mm 38748/ | 524.8 | P | 588.4 | P | 507.4 | P | 357.4 |
| | | len = 1310/STRA = for | | | | | | | |
| 11 | 99115_at | Cluster Incl AW046350: UI-M-BH1-ali-a-01-0-UI s1 Mus musculus cDNA, 3 end/clone = UI-M-BH1-ali-a-01-0-UI/ | 2517 | P | 2457.7 | P | 2521.8 | P | 2606.1 |
| | | clone_end = 3/gb = AW046350/gi = 5906879/ug = Mm 400/len = 253/STRA = for | | | | | | | |
| 11 | 99164_at | Cluster Incl AI844560 UI-M-AJ1-ahg-c-05-0-UI s2 Mus musculus cDNA, 3 end/clone = UI-M-AJ1-ahg-c-05-0-UI/ | 409.4 | P | 424.9 | P | 462.5 | P | 343.7 |
| | | clone_end = 3/gb = AI844560/gi = 5488466/ug = Mm 41680/len = 357/STRA = for | | | | | | | |
| 11 | 99175_at | Cluster Incl AI843396 UI-M-AQ1-adz-g-07-0-UI s1 Mus musculus cDNA, 3 end/clone = UI -M-AQ1-adz-g-07-0-UI/ | 647.5 | P | 777.7 | P | 782.1 | P | 586.5 |
| | | clone_end = 3/gb = AI843396/gi = 5477609/ug = Mm 41780/len = 289/STRA = for | | | | | | | |
| 11 | 99584-at | Cluster Incl D14883 CD82 anligen/cds = (102,902)/gb = D14883/gi = 984036/ug = Mm 4261/len = 1657/STRA = for | 625.7 | P | 555.9 | P | 620.9 | P | 462.9 |
| 11 | 99599_s_at | Cluster Incl AW210320: ul53e09 y1 Mus musculus cDNA, 5 end/clone = IMAGE-2102056/clone_end = 5/gb = AW210320/ | 1575 | P | 2107.1 | P | 2191.7 | P | 1230 |
| | | gi = 6516260/ug = Mm 42855/len = 477/STRA = for | | | | | | | |
| 11 | 99629_at | Cluster Incl AW060710: UI-M-BH1-anl-g-04-0-UI s1 Mus musculus cDNA, 3 end/clone = UI -M-BH1-anl-g-04-0-UI/ | 976.9 | P | 1119.9 | P | 1387 | P | 955.2 |
| | | clone_end = 3/gb = AW060710/gi = 6008461/ug = Mm 4337/len = 535/STRA = for | | | | | | | |
| 11 | 99651_at | Cluster Incl AI849549 UI-M-AH1-agr-a-10-0-UI s1 Mus musculus cDNA, 3 end/clone = UI-M-AH1-agr-a-10-0-UI/ | 555.8 | P | 409.2 | P | 550.2 | P | 439.2 |
| | | clone end = 3/gb = AI849549/gi = 5493455/ug = Mm 43737/len = 279/STRA = for | | | | | | | |
| 11 | 99665_at | Cluster Incl U05252 Mus musculus nuclear matrix attachment DNA-binding protein SATB1 mRNA, complete cds/ | 469.7 | P | 688.3 | P | 596.2 | P | 393.3 |
| | | cds = (198,2492)/gb = U05252/gi = 453370/ug = Mm 4381/len = 2922/STRA = for | | | | | | | |
| 11 | 100039_at | Cluster Incl AW125880 UI-M-BH2 2-aqk-g-03-0-UI s1 Mus musculus cDNA, 3 end/clone = UI-M-BH2 2-aqk-g-03-0-UI/ | 431.8 | P | 607.7 | P | 595.6 | P | 286.2 |
| | | clone_end = 3/gb = AW125880/gi = 6101410/ug = Mm 44220/len = 490/STRA = for | | | | | | | |
| 11 | 100153_at | Cluster Incl X15052 Neural cell adhesion molecule/cds = (0,1246)/gb = X15052/gi = 53349/ug = Mm 4974/len = 4552/ | 561.4 | P | 601.8 | P | 718.1 | P | 418.9 |
| | | STRA = for | | | | | | | |
| 11 | 100538_at | Cluster Incl M35725 Mouse Cu-Zn superoxide dismutase mRNA, complete cds/cds(4,468)/gb = M35725/gi = 192929/ | 1610.4 | P | 1873.2 | P | 1754.7 | P | 1471.5 |
| | | ug = Mm 5274/len = 469/STRA = for | | | | | | | |
| 11 | 100540_at | Cluster Incl M63848: Mouse leukotnene A-4 hydrolase mRNA, complete cds/cds = (80,1915)/gb = M63848/gi = 198883/ | 927.2 | P | 894.9 | P | 1044.4 | P | 756.5 |
| | | ug-Mm 533/len = 2039/STRA = for | | | | | | | |
| 11 | 100576_at | Cluster Incl U57746: Mus musculus platelet-activating, factor acetylhydrolase isoform lb gamma subunit (PAF-AH gamma) | 473 | P | 556.1 | P | 604.5 | P | 424.9 |
| | | mRNA, complete cds/cds = (129,627)/gb = U57746/gi = 1373360/ug = Mm 597/len = 872/STRA = for | | | | | | | |
| 11 | 100629_at | Cluster Incl U24428 Mus musculus mu-class glutathione s-transferase (mGSTM5) mRNA, complete cds/cds = (96,770)/ | 488.2 | P | 578.5 | P | 667.3 | P | 438.9 |
| | | gb = U24428/gi = 1002385/ug = Mm.667/len = 907/STRA = for | | | | | | | |
| 11 | 101000_at | Cluster Incl D78643 Mouse seizure-related mRNA, partial cds/cds = (0,422)/gb = D78643/gi = 1304167/ug = Mm 675/ | 567.7 | P | 604.8 | P | 575 | P | 488 |
| | | len = 1492/STRA = for | | | | | | | |
| 11 | 101007_at | Cluster Incl AI845732 UI-M-AQ1-aeb-b-04-0-UI s1 Mus musculus cDNA, 3 end/clone = UI-M-AQ1-aeb-b-04-0-UI/ | 869 | P | 1033.3 | P | 1544.5 | P | 835.6 |
| | | clone_end = 3/gb = AI845732/gi = 5489638/ug = Mm 6797/len = 184/STRA = for | | | | | | | |
| 11 | 101013_at | Cluster Incl U52623 Mus musculus omithine decarboxylase anlizyme gene, complete cds/cds = (0,683)/gb = U52823/ | 3829.9 | P | 3866.7 | P | 3930.2 | P | 3649.4 |
| | | gi = 1924975/ug = Mm 683/len = 684/STRA = for | | | | | | | |
| 11 | 101042_f_at | Cluster Incl U51014 Peptidase 4/cds = (54,1535)/gb = U51014/gi = 1236705/ug = Mm 69751/len = 1876/STRA = for | 317.9 | P | 526.5 | P | 521.3 | P | 339.6 |
| 11 | 101053_at | Cluster Incl AW046847: UI-M-BH1-alz-h-03-0-UI s1 Mus musculus cDNA, 3 end/clone = UI-M-BH1-alz-h-03-0-UI/ | 638.2 | P | 666.9 | P | 771.3 | P | 595.9 |
| | | clone_end = 3/gb = AW046847/gi = 5907376/ug = Mm 7039/len = 242/STRA = for | | | | | | | |
| 11 | 101059_at | Cluster Incl D76440 Necdin/cds = (86,1063)/gb = D76440/gi = 1040690/ug = Mm 7089/len = 1608/STRA = for | 506.8 | P | 746.3 | P | 759.6 | P | 397.4 |
| 11 | 101072_at | Cluster Incl AW124250 UI-M-BH2.1-aph-f-11-0-UI s1 Mus musculus cDNA, 3 end/clone = UI-M-BH2.1-aph-f-11-0-UI/ | 1980.9 | P | 2100.9 | P | 2532.5 | P | 1899.4 |
| | | clone_end = 3/gb = AW124250/gi = 6099780/ug = Mm 7215/len = 267/STRA = for | | | | | | | |
| 11 | 101080_at | Cluster Incl AB009993 Mus musculus mRNA for collagen a1(V), complete cds/cds = (131,5647)/gb = AB009993/ | 135.1 | P | 389.6 | P | 692.4 | P | 167.9 |
| | | gi = 3219171/ug = Mm 7281/len = 6156/STRA = for | | | | | | | |
| 11 | 101495_at | Cluster Incl X59047 M musculus MD3 mRNA/cds = UNKNOWN/gb = X59047/gi = 53037/ug = Mm 806/len = 1234/STRA = for | 1022.5 | P | 1293.2 | P | 1216.1 | P | 1031.2 |
| 11 | 101589_at | Cluster Incl X12944 Mouse mRNA for HMG-17 chromosomal protein/cds = (39,311)/gb = X12944/gi = 51336/ug = Mm 911/ | 2039.2 | P | 2388.5 | P | 2402.4 | P | 1864.4 |
| | | len = 1113/STRA = for | | | | | | | |
| 11 | 101976_at | Cluster Incl AF071314: Mus musculus COP9 complex subunit 4 (COPS4) mRNA, complete cds/cds = (37,1257)/ | 527.7 | P | 445.8 | P | 471.3 | P | 446.7 |
| | | gb = AF071314/gi = 3309169/ug = Mm 957/len = 1620/STRA = for | | | | | | | |
| 11 | 102003_at | Cluster Incl AI842159 UI-M-AN1-afj-f-10-0-UI.s1 Mus musculus cDNA, 3 end/clone = UI-M-AN1-afj-f-10-0-UI/clone_end = 3/ | 409 | P | 324.2 | P | 388.4 | P | 268.8 |
| | | gb = AI842159/gi = 5476372/ug = Mm 99132/len = 277/STRA = for | | | | | | | |
| 11 | 103332_at | Cluster Incl AI197507 ud56b04 r1 Mus musculus cDNA, 5 end/clone = IMAGE-1449871/clone_end = 5/gb = AI197507/ | 123.5 | P | 96.5 | P | 142 | P | 124.3 |
| | | gi = 3750113/ug = Mm 18062/len = 436/STRA = for | | | | | | | |
| 11 | 103591_at | Cluster Incl D49439 Mouse mRNA for TFIID subunit p80, complete cds/cds = (10,2046)/gb = D49439/gi = 1372970/ | 223.5 | P | 224.4 | P | 244.2 | P | 224.8 |
| | | ug = Mm 1994/len = 2192/STRA = for | | | | | | | |
| 11 | 103642_at | Cluster Incl AB001927: Mus musculus mRNA for ras-GTPase-activating protein SH3-domain binding protein, complete cds/ | 1714.8 | P | 1822.4 | P | 1816.2 | P | 1395.8 |
| | | cds = (68,1465)/gb = AB001927/gi = 1902906/Ug = Mm.2038/len = 2686/STRA = for | | | | | | | |
| 11 | 103656_at | Cluster Incl Y16518 Mus musculus mRNA for G protein-coupled receptor, P40GPRT/cds = (257,1456)/gb = Y16518/ | 619.8 | P | 612.1 | P | 732.6 | P | 507.5 |
| | | gi = 3492792/ug = Mm 20522/len = 1581/STRA = for | | | | | | | |
| 11 | 103817_at | Cluster Incl AJ006469 Mus musculus mRNA for cartilage-associated protein (CASP)/cds = (71,1273)/gb = AJ006469/ | 1216.4 | P | 1622.3 | P | 1540.9 | P | 1058.7 |
| | | gi = 3687319/us = Mm.20904/len = 1685/STRA = for | | | | | | | |
| 11 | 103837_at | Cluster Incl AF092997: Mus musculus caspase-14 mRNA, complete cds/cds = (60,833)/gb = AF092997/gi = 3719426/ | 1855.2 | P | 1994.5 | P | 2831 | P | 984.1 |
| | | ug = Mm 20940/len = 2175/STRA = for | | | | | | | |
| 11 | 103858_at | Cluster Incl AW124023: UI-M-BH2.1-apn-b-12-0-UI.s1 Mus musculus cDNA, 3 end/clone = UI-M-BH2.1-apn-b-12-0-UI/ | 209.5 | P | 88.5 | P | 240.9 | P | 178.5 |
| | | clone_end = 3/gb = AW124023/gi = 6099553/ug = Mm.21096/len = 436/STRA = for | | | | | | | |
| 11 | 104118_at | Cluster Incl AW123781 UI-M-BH2 1-apm-c-11-0-UI s1 Mus musculus cDNA, 3 end/clone = UI-M-BH2.1-apm-c-11-0-UI/ | 410.2 | P | 480.5 | P | 474.2 | P | 363.4 |
| | | clone_end = 3/gb = AW123781/gi = 6099311/ug = Mm.21972/len = 396/STRA = for | | | | | | | |
| 11 | 104143_at | Cluster Incl AI843212 UI-M-AK1-aet-f-07-0-UI s1 Mus musculus cDNA, 3 end/clone = UI-M-AK1-aet-f-07-0-UI/ | 593.5 | P | 648.1 | P | 727.7 | P | 508.1 |
| | | clone_end = 3/gb = AI843212/gi = 5477425/ug = Mm 22144/len = 368/STRA = for | | | | | | | |
| 11 | 104150_at | Cluster Incl AW121957 UI-M-BH2 3-aoi-h-06-0-UI s1 Mus musculus cDNA, 3 end/clone = UI-M-BH2 3-aoi-h-06-0-UI/ | 436.5 | P | 532.1 | P | 403.8 | P | 350 |
| | | clone_end = 3/gb = AW121957/gi = 6097308/ug = Mm 22189/len = 274/STRA = for | | | | | | | |
| 11 | 104376_at | Cluster Incl AF006602 Mus musculus histone deacetylase mHDA1 mRNA, complete cds/cds = (0,2975)/gb = AF006602/ | 773.6 | P | 924.3 | P | 887.6 | P | 624.4 |
| | | gi = 4165859/ug = Mm 22665/len = 3081/STRA = for | | | | | | | |
| 11 | 104390_at | Cluster Incl AI836916: UI-M-AJ0-aav-c-12-0-UI.s1 Mus musculus cDNA, 3 end/clone = UI-M-AJ0-aav-c-12-0-UI/ | 637.3 | P | 582.5 | P | 509 | P | 462.6 |
| | | clone/end = 3/gb = AI836916/gi = 5471129/ug = Mm 22717/len = 489/STRA = for | | | | | | | |
| 11 | 104623_at | Cluster Incl X73360 M. musculus mESG mRNA/cds = (258,2573)/gb = X73360/gi = 313235/ug = Mm 24255/len = 2840/ | 517.8 | P | 740.9 | P | 657.4 | P | 537.8 |
| | | STRA = for | | | | | | | |
| 11 | 104624_at | Cluster Incl AI851539 UI-M-BH0-aki-c-09-0-UI.s1 Mus musculus cDNA, 3 end/clone = UI-M-BH0-aki-c-09-0-UI/ | 838.6 | P | 889 | P | 1128.1 | P | 801.8 |
| | | clone_end = 3/gb = AI851539/gi = 5495445/ug = Mm.24260/len = 530/STRA = for | | | | | | | |
| 11 | 104715_at | Cluster Incl AI152779 ud91a09 r1 Mus musculus cDNA, 5 end/clone = IMAGE-1478200/clone_end = 5/gb = AI152779/ | 1221.8 | P | 1167.3 | P | 1107.4 | P | 913.7 |
| | | gi = 3681248/ug = Mm.24496/len = 504/STRA = for | | | | | | | |
| 11 | 94330_at | Cluster Incl AA710564 vI43b11 r1 Mus musculus cDNA, 5 end/clone = IMAGE-1165821/clone_end = 5/gb = AA710564/ | 562.1 | P | 1082.7 | P | 934.6 | P | 428.2 |
| | | gi = 2720482/ug = Mm.24887/len = 519/STRA = for | | | | | | | |
| 11 | 94384_at | Cluster Incl AI847926 UI-M-AI1-afl-c-08-0-UI.s1 Mus musculus cDNA, 3 end/clone = UI-M-AI1-afl-c-08-0-UI/clone_end = 3/ | 1309.3 | P | 1327.5 | P | 1225.1 | P | 950.5 |
| | | gb = AI847926/gi = 5491827/ug = Mm 25279/len = 446/STRA = for | | | | | | | |
| 11 | 94410_f_at | Cluster Incl AA611413: vo51e11 r1 Mus musculus cDNA, 5 end/clone = IMAGE-1053452/clone_end = 5/gb = AA611413/ | 890.3 | P | 957.4 | P | 913.3 | P | 822.1 |
| | | gi = 2461492/ug = Mm 25941/len = 402/STRA = rev | | | | | | | |
| 11 | 94932_at | Cluster Incl M29464 Platelet derrved growth factor, alpha/cds = (62,652)/gb = M29464/gi = 200272/ug = Mm 2675/len = 906/ | 1366.5 | P | 2056.3 | P | 1987.9 | P | 1363.9 |
| | | STRA = for | | | | | | | |
| 11 | 95629_at | Cluster Incl AI843679: UI-M-AK1-aer-c-01-0-UI s1 Mus musculus cDNA, 3 end/clone = UI-M-AK1-aer-c-01-0-UI/ | 575.7 | P | 678.7 | P | 690.2 | P | 379 |
| | | clone_end = 3/gb = AI843679/gi = 5477892/ug = Mm 28073/len = 184/STRA = for | | | | | | | |
| 11 | 95636_at | Cluster Incl AW123628 UI-M-BH2 1-api-e-09-0-UI s1 Mus musculus cDNA, 3 end/clone = UI-M-BH2 1-api-e-09-0-UI/ | 1383.4 | P | 1623.8 | P | 1637.5 | P | 885.4 |
| | | clone_end = 3/gb = AW123628/gi = 6099158/ug = Mm 28094/len = 416/STRA = for | | | | | | | |
| 11 | 96195_at | Cluster Incl U57686 Mus musculus p130Cas-related protein Sin mRNA, complete cds/cds = (57,1739)/gb = U57686/ | 720.8 | P | 564.3 | P | 625.3 | P | 394.1 |
| | | gi = 1373343/ug = Mm 28543/len = 2211/STRA = for | | | | | | | |
| 11 | 96236_at | Cluster Incl AW122965 UI-M-BH2 1-aoz-g-11-0-UI s2 Mus musculus cDNA, 3 end/clone = UI-M-BH2 1-aoz-g-11-0-UI/ | 389.3 | P | 471.7 | P | 691.2 | P | 350.1 |
| | | clone_end = 3/gb = AW122965/gi = 6098495/ug = Mm 28798/len = 620/STRA = for | | | | | | | |
| 11 | 96791 _at | Cluster Incl AW047875 UI-M-BH1-als-g-03-0-UI s1 Mus musculus cDNA, 3 end/clone = UI-M-BH1-als-g-03-0-UI/ | 919.3 | P | 937.2 | P | 788 | P | 939.7 |
| | | clone end = 3/gb = AW047875/gi = 5908404/ug = Mm 29116/len = 324/STRA = for | | | | | | | |
| 11 | 97330_at | Cluster Incl AI840499 UI-M-AN0-aci-g-08-0-UI.s1 Mus musculus cDNA, 3 end/clone = UI-M-AN0-aci-g-08-0-UI/ | 1627.4 | P | 1917.4 | P | 1955.6 | P | 1545.9 |
| | | clone_end = 3/gb = AI840499/gi = 5474712/ug = Mm 29543/len = 426/STRA = for | | | | | | | |
| 11 | 97386_at | Cluster Incl AI853294 UI-M-BH0-aji-f-03-0-UI s1 Mus musculus cDNA, 3 end/clone = UI-M-BH0-aji-f-03-0-UI/clone_end = 3/ | 802.6 | P | 820.8 | P | 1036.6 | P | 618.2 |
| | | gb = AI853294/gi = 5497200/ug = Mm 29789/len = 413/STRA = for | | | | | | | |
| 11 | 97407_at | Cluster Incl AI848025 UI-M-AH1-agp-e-07-0-UI s1 Mus musculus cDNA, 3 end/clone = UI-M-AH1-agp-e-07-0-UI/ | 183.6 | P | 339.7 | P | 322.1 | P | 191.5 |
| | | clone_end = 3/gb = AI848025/gi = 5491931/ug = Mm 29926/len = 435/STRA = for | | | | | | | |
| 11 | 97415_at | Cluster Incl M89777 Mus musculus GTP-binding protein (Rab3D) mRNA, complete cds/cds = (43,702)/gb = M89777/ | 646 | P | 709.9 | P | 821 | P | 464.6 |
| | | gi = 200631/ug = Mm.29968/len = 703/STRA = for | | | | | | | |
| 11 | 97925_at | Cluster Incl AB026241.Mus musculus mRNA for casein kinase, complete cds/cds = (88,1338)/gb = AB028241/gi = 6526519/ | 333.7 | P | 391.9 | P | 343.1 | P | 241.2 |
| | | ug = Mm 30199/len = 1392/STRA = for | | | | | | | |
| 11 | 97953 g_at | Cluster Incl U39818 Tuberous sclerosis 2/cds = (90,5534)/gb = U39818/gi = 1236401/ug = Mm 30435/1en = 5637/STRA = for | 91.1 | P | 159.7 | P | 173 | P | 60.8 |
| 11 | 98440_at | Cluster Incl AA596710 vm60d08 r1 Mus musculus cDNA, 5 end/clone = IMAGE-1002639/clone_end = 5/gb = AA596710/ | 754.8 | P | 837.7 | P | 1157.8 | P | 387 |
| | | gi = 2412145/ug = Mm 34497/len = 396/STRA = for | | | | | | | |
| 11 | 98447_at | Clusler Incl M62362.CCAAT/enhancer binding protein (C/EBP), alpha/cds = (0,1187)/gb = M62362/gi = 192381/ | 3335.2 | P | 3682 | P | 3866.2 | P | 3122 |
| | | ug = Mm 34537/len = 1188/STRA = rev | | | | | | | |
| 11 | 98473_at | Cluster Incl AF032466: Mus musculus arginase II mRNA, complete cds/cds = (34,1098)/gb = AF032466/gi = 2642321/ | 303.9 | P | 278.3 | P | 315.7 | P | 233.3 |
| | | ug = Mm 3506/len = 1363/STRA = for | | | | | | | |
| 11 | 99926_at | Cluster Incl AB001489: Phosphatidylinositol glycan, class R/cds = (0,2315)/gb = AB001489/gi = 2804245/ug = Mm 4317/ | 694.7 | P | 474.3 | P | 698.5 | P | 446 |
| | | len = 3733/STRA = for | | | | | | | |
| 11 | 100410_at | Cluster Incl AW122781 UI-M-BH2 1-aoy-d-01-0-UI.s1 Mus musculus cDNA, 3 end/clone = UI-M-BH2 1-aoy-d-01-0-UI/ | 290.2 | P | 379.8 | P | 336.2 | P | 224.9 |
| | | clone_end = 3/gb = AW122781/gi = 6098311/ug = Mm 46641/len = 162/STRA = for | | | | | | | |
| 11 | 100890_at | Cluster Incl AI173038 uh16b06.r1 Mus musculus cDNA, 5 end/clone = IMAGE-1745555/clone_end = 5/gb = AI173038/ | 630 | P | 688.9 | P | 582.9 | P | 522.6 |
| | | gi = 3720618/ug = Mm 5458/len = 431/STRA = for | | | | | | | |
| 11 | 100927_at | Cluster Incl U28960 Phospholipid transfer protein/cds = (0,1475)/gb = U28960/gi = 902887/ug = Mm.6105/len = 1590/ | 319.5 | P | 399.6 | P | 404.8 | P | 160 |
| | | STRA = for | | | | | | | |
| 11 | 100948_at | Cluster Incl AW049351: UI-M-BH1-amr-h-10-0-UI s1 Mus musculus cDNA, 3 end/clone = UI-M-BH1-amr-h-10-0-UI/ | 801.7 | P | 917.5 | P | 1163.5 | P | 626 |
| | | clone end = 3/gb = AW049351/gi = 5909880/ug = Mm 6403/len = 318/STRA = for | | | | | | | |
| 11 | 101430_at | Cluster Incl AW124153 UI-M-BH2.1-apr-10-0-UI s1 Mus musculus cDNA, 3 end/clone = UI-M-BH2 1-apr-f-10-0-UI/ | 766.5 | P | 867.4 | P | 855.4 | P | 588.8 |
| | | clone_end = 3/gb = AW124153/gi = 6099683/ug = Mm 7556/len = 219/STRA = for | | | | | | | |
| 11 | 101441_i_at | Cluster Incl AF031127 Mus musculus inositol trisphosphate receptor type 2 (Itpr2) gene, partial cds/cds = (0,110)/ | 519.2 | P | 431.2 | P | 897.2 | P | 444.2 |
| | | gb = AF031127/gi = 2911341/ug = Mm 7800/len = 111/STRA = rev | | | | | | | |
| 11 | 101462_r_at | Cluster Incl U06944 Praja1/cds = (328,1602)/gb = U06944/gi = 2828789/ug = Mm 8211/len = 1978/STRA = for | 220.9 | P | 279.5 | P | 213.4 | P | 193.6 |
| 11 | 101930_at | Cluster Incl Y07688: M musculus mRNA for NfiX1-protein/cds = (316,1641)/gb = Y07688/gi = 1524166/ug = Mm 9394/ | 1595.3 | P | 2167.3 | P | 2151.3 | P | 1662.1 |
| | | len = 1729/STRA = for | | | | | | | |
| 11 | 103485_at | Cluster Incl AA795415: vv06f02.r1 Mus musculus cDNA, 5 end/clone = IMAGE-1210875/clone_end = 5/gb = AA795415/ | 43.3 | P | 107.5 | P | 177.3 | P | 120.7 |
| | | gi = 2858370/ug = Mm.22139/len = 298/STRA = for | | | | | | | |
| 11 | 103921_i_at | Cluster Incl AI839690 UI-M-AN0-acp-e-04-0-UI.s1 Mus musculus cDNA, 3 end/clone = UI-M-AN0-acp-e-04-0-UI/ | 470.7 | P | 520.1 | P | 418 | P | 337.9 |
| | | clone_end = 3/gb = AI839690/gi = 5473903/ug = Mm 25034/len = 250/STRA = rev | | | | | | | |
| 11 | 103924_at | Cluster Incl AW048884 UI-M-BH1-amf-f-03-0-UI.s1 Mus musculus cDNA, 3 end/clone = UI-M-BH1-amf-f-03-0-UI/ | 755.3 | P | 709.4 | P | 806.1 | P | 356.2 |
| | | clone_end = 3/gb = AW048884/gi = 5909413/ug = Mm.25078/len = 251/STRA = rev | | | | | | | |
| 11 | 104016_at | Cluster Incl M91236: Gap junction membrane channel protein beta 5/cds = (120,935)/gb = M91236/gi = 192642/ | 581.2 | P | 617.4 | P | 690.3 | P | 303.8 |
| | | ug = Mm 26859/len = 1272/STRA = for | | | | | | | |
| 11 | 104025_at | Cluster Incl AW047185 UI-M-BH1-amb-a-12-0-UI s1 Mus musculus cDNA, 3 end/clone = UI-M-BH1-amb-a-12-0-UI/ | 565.9 | P | 675 | P | 687.3 | P | 381.4 |
| | | clone_end = 3/gb = AW047185/gi = 5907714/ug = Mm.26995/len = 416/STRA = for | | | | | | | |
| 11 | 104207_at | Cluster Incl AI430272 mf46f12 y1 Mus musculus cDNA, 5 end/clone = IMAGE-408143/clone_end = 5/gb = AI430272/ | 235.5 | P | 159.7 | P | 284 | P | 208.2 |
| | | gi = 4276108/ug = Mm 27654/len = 426/STRA = for | | | | | | | |
| 11 | 104232_at | Cluster Incl X63099 Gap junction membrane channel protein beta 3/cds = (119,931)/gb = X63099/gi = 50598/ug = Mm 28078/ | 1050.4 | P | 1244.3 | P | 1190.9 | P | 831 |
| | | len = 1636/STRA = for | | | | | | | |
| 11 | 104255_at | Cluster Incl AA795285 vv03h09 r1 Mus musculus cDNA, 5 end/clone = IMAGE-1210625/clone_end = 5/gb = AA795285/ | 300 | P | 255.6 | P | 262 | P | 197.8 |
| | | gi = 2858240/ug = Mm 28387/len = 297/STRA = rev | | | | | | | |
| 11 | 104400_at | Cluster Incl AF076956 Mus musculus prenylaied SNARE protein Ykt6 (Ykt6) mRNA, complete cds/cds = (15,611)/ | 193.9 | P | 137.7 | P | 196.1 | P | 176.5 |
| | | gb = AF076956/gi = 3328384/ug = Mm 28680/len = 643/STRA = for | | | | | | | |
| 11 | 104434_at | Cluster Incl AW048552 UI-M-BH1-alx-d-07-0-UI s1 Mus musculus cDNA, 3 end/clone = UI-M-BH1-alx-d-07-0-UI/ | 866.6 | P | 613.6 | P | 717.2 | P | 663.2 |
| | | clone_end = 3/gb = AW048552/gi = 5909081/ug = Mm 29241/len = 473/STRA = for | | | | | | | |
| 11 | 92310_at | Cluster Incl M96163 Mus musculus (clone 2) serum inducible kinase (SNK) mRNA, mRNA sequence/cds = UNKNOWN/ | 1126 | P | 1026.5 | P | 1069.4 | P | 875 |
| | | gb = M96163/gi = 200999/ug = Mm 380/len = 2772/STRA = for | | | | | | | |
| 11 | 92517_at | Cluster Incl AB026583 Mus musculus mRNA for Scr3, complete cds/cds = (34,1104)/gb = AB026583/gi = 4730922/ | 101 | P | 105.3 | P | 175.5 | P | 89.1 |
| | | ug = Mm 42247/len = 1354/STRA = for | | | | | | | |
| 11 | 92726_at | Cluster Incl AJ010605 SRY-box containing gene 6/cds = (0,931)/gb = AJ010605/gi = 3702611/ug = Mm 4656/len = 3371/ | −25.3 | P | 44 | P | 85.2 | P | 28.8 |
| | | STRA = for | | | | | | | |
| 11 | 93437_f _at | Cluster Incl AI850509 UI-M-BG1-aih-h-10-0-UI s1 Mus musculus cDNA, 3 end/clone = UI-M-BG1-aih-h-10-0-UI/ | 108.3 | P | 130.2 | P | 125.2 | P | 38.4 |
| | | clone_end = 3/gb = AI850509/gi = 5494415/ug = Mm 65832/len = 453/STRA = for | | | | | | | |
| 11 | 93467_at | Cluster Incl AI852734. UI-M-BH0-aji-h-02-0-UI s1 Mus musculus cDNA, 3 end/clone = UI-M-BH0-aji-h-02-0-UI/clone_end = 3/ | 351.6 | P | 381.6 | P | 256.5 | P | 230.1 |
| | | gb = AI852734/gi = 5496640/ug = Mm 6926/len = 348/STRA = for | | | | | | | |
| 11 | 93697_at | Cluster Incl U63387 Chromobox homolog 4 (Drosophila Pc class)/cds = (132,1787)/gb = U63387/gi = 2781391/ug = Mm 8556/ | 382.6 | P | 333.8 | P | 436.6 | P | 289.2 |
| | | len = 2542/STRA = for | | | | | | | |
| 11 | 94789_r_at | Cluster Incl X04663: Mouse mRNA for beta-tubulin (isotype Mbeta 5)/cds = (78,1412)/gb = X04663/gi = 55047/ug = Mm. 1703/ | 2627.7 | P | 2559.8 | P | 2762.5 | P | 2388.7 |
| | | len = 1591/STRA = for | | | | | | | |
| 11 | 95952_at | Cluster Incl AW124781: UI-M-BH2.1-aqa-h-01-0-UI s1 Mus musculus cDNA, 3 end/clone = UI-M-BH2 1-aqa-h-01-0-UI/ | 401.5 | P | 480 | P | 430.5 | P | 259.1 |
| | | clone_end = 3/gb = AW124781/gi = 6100311/ug = Mm 24921/len = 422/STRA = rev | | | | | | | |
| 11 | 99872_s_at | Cluster Incl L39879: Ferritin light chain 1/cds = (205,756)/gb = L39879/gi = 666913/ug = Mm 7500/len = 920/STRA = for | 3676.8 | P | 4096.8 | P | 4252.1 | P | 3860.8 |
| 11 | 100331_g_at | Cluster Incl AF032714 Mus musculus type II peroxiredoxin protein 2 (PrxII-2) pseudogene, complete sequence/ | 3889.8 | P | 4064.5 | P | 4081.8 | P | 3063.5 |
| | | cds = (0,596)/gb = AF032714/gi = 3603231/ug = Mm 87831/len = 597/STRA = for | | | | | | | |
| 11 | 100342_l_at | Cluster Incl M28729: Tubulin alpha 1/cds = (0,596)/gb = M28729/gi = 202222/ug = Mm 88110/len = 786/STRA = for | 5045.1 | P | 5495.7 | P | 5142.2 | P | 4406 |
| 11 | 95232_at | Cluster Incl AB009392 Mus musculus mRNA for protein L, partial cds/cds = (0,600)/gb = AB009392/gi = 2723394/ | 1539.8 | P | 1731.1 | P | 1829.2 | P | 1487.5 |
| | | ug = Mm 9043/len = 958/STRA = for | | | | | | | |
| 11 | 95795_at | Cluster Incl U96810 Mus musculus Supt4h2 gene, complete cds/cds = (0,353)/gb = U96810/gi = 3779195/ug = Mm 87825/ | 585.7 | P | 617.5 | P | 756.7 | P | 632.7 |
| | | len = 354/STRA = for | | | | | | | |
| 11 | 96426_at | Cluster Incl U38967 Prothymosin beta 4/cds = (0,134)/gb = U38967/gi = 1072099/ug = Mm 22566/len = 542/STRA = for | 2993.2 | P | 3016.2 | P | 3123.6 | P | 2868.4 |
| 11 | 101123_at | Cluster Incl U76253: Integral membrane protein 2 B/cds = (145,945)/gb = U76253/gi = 3746126/ug = Mm 4266/len = 1790/ | 1939.8 | P | 2488.8 | P | 3241.2 | P | 1164.3 |
| | | STRA = for | | | | | | | |
| 11 | 101607_at | Cluster Incl C80068 C80068 Mus musculus cDNA, 3 end/clone = J0076B01/clone_end = 3/gb = C80068/gi = 2520398/ | 250.8 | P | 301.5 | P | 257.4 | P | 188.9 |
| | | ug = Mm 5905/len = 287/STRA = rev | | | | | | | |
| 11 | 102134_f_at | Cluster Incl AI461702 vw57d12.x1 Mus musculus cDNA, 3 end/clone = Image-1247927/clone_end = 3/gb = AI461702/ | 736.3 | P | 828.9 | P | 816.4 | P | 581.4 |
| | | gi = 4315732/ug = Mm.43556/len = 470/STRA = rev | | | | | | | |
| 11 | 92610_at | Cluster Incl M21332 Mouse MHC class III RD gene (H2-d and H2-Sk haplotypes), complete cds/cds = (0,1127)/gb = M21332/ | 429.5 | P | 402 | P | 410.4 | P | 305.6 |
| | | gi = 199607/ug = Mm.10964/len = 1128/STRA = for | | | | | | | |
| 11 | 93300_at | Cluster Incl X57413 Transforming growth factor, beta 2/cds = (1217,2461)/gb = X57413/gi = 54772/ug = Mm 18213/len = 4267/ | 86.4 | P | 82.1 | P | 82.1 | P | −8 |
| | | STRA = rev | | | | | | | |
| 11 | 93598_at | Cluster Incl AI840437 UI-M-AN0-aci-a-04-0-UI s1 Mus musculus cDNA, 3 end/clone = UI-M-AN0-aci-04-0-UI/ | 133.2 | P | 92.2 | P | 108.6 | P | 81.2 |
| | | clone_end = 3/gb = AI840437/gi = 5474650/ug = Mm.20847/len = 287/STRA = for | | | | | | | |
| 11 | 93967_at | Cluster Incl AI850979 UI-M-BH0-akd-h-04-0-UI.s1 Mus musculus cDNA, 3 end/clone = UI-M-BH0-akd-h-04-0-UI/ | 754.2 | P | 730.6 | P | 612.8 | P | 560 |
| | | clone_end = 3/gb = AI850979/gi = 5494885/ug = Mm.21645/len = 216/STRA = for | | | | | | | |
| 11 | 94277_at | Cluster Incl U66257. Mus musculus thrombospodin 3 and metaxin genes, exon 1, partial cds, and intergenic region/ | 307.3 | P | 266.6 | P | 212.7 | P | 196.1 |
| | | cds = (0,81)/gb = U66257/gi = 1575552/ug = Mm 22508/len = 82/STRA = for | | | | | | | |
| 11 | 94807_at | Cluster Incl AI848354 UI-M-AH1-ago-h-07-0-UI s1 Mus musculus cDNA, 3 end/clone = UI-M-AH1-ago-h-07-0-UI/ | 793.4 | P | 939.8 | P | 997.8 | P | 742.5 |
| | | clone_end = 3/gb AI848354/gi = 5492300/ug = Mm 22679/len = 221/STRA = for | | | | | | | |
| 11 | 95512_at | Cluster Incl M60320 Protein-L-isoaspartate (D-aspartate) O-Methyltransferase 1/cds = (119,802)/gb = M60320/gi = 200254/ | 127.8 | P | 151.8 | P | 94.7 | P | 123.5 |
| | | ug = Mm 25293/len = 1580/STRA = for | | | | | | | |
| 11 | 97807_at | Cluster Incl AI838630 UI-M-AO0-aca-a-04-0-UI s1 Mus musculus cDNA, 3 end/clone = UI-M-AO0-aca-a-04-0-UI/ | 184.9 | P | 245.6 | P | 266.9 | P | 59.7 |
| | | clone_end = 3/gb = AI838630/gi = 5472843/ug = Mm.2815/len = 430/STRA = for | | | | | | | |
| 11 | 98938_at | Cluster Incl AI852184 UI-M-BH0-ajc-a-05-0-UI s1 Mus musculus cDNA, 3 end/clone = UI-M-BHo-ajc-a-05-0-UI/ | 338.8 | P | 346.4 | P | 294.1 | P | 301.5 |
| | | clone_end = 3/gb = AI852184/gi = 5496090/ug = Mm 28690/len = 258/STRA = for | | | | | | | |
| 11 | 96873_at | Cluster Incl AI843287: UI-M-AO1-aei-e-06-0-UI s1 Mus musculus cDNA, 3 end/clone = UI-M-AO1-aei-e-06-0-UI/ | 575.1 | P | 479.3 | P | 499.9 | P | 426.2 |
| | | clone_end = 3/gb = AI843287/gi = 5477500/ug = Mm 30069/len = 425/STRA = for | | | | | | | |
| 11 | 96939_at | Cluster Incl AI842649: UI-M-AQ1-aed-g-07-0-UI.s1 Mus musculus cDNA, 3 end/clone = UI-M-AQ1-aed-g-07-0-UI/ | 454.4 | P | 367.5 | P | 466 | P | 222.4 |
| | | clone_end = 3/gb = AI842649/gi = 5476862/ug = Mm.30188/len = 312/STRA = for | | | | | | | |
| 11 | 102327_at | Cluster Incl AF078705: Amine oxidase, copper containing 3/cds = (0,2297)/gb = AF078705/gi = 3603354/ug = Mm 10734/ | 356.3 | P | 376.5 | P | 360.9 | P | 219.1 |
| | | len = 2298/STRA = for | | | | | | | |
| 11 | 102783_at | Cluster Incl AI131744 ue24h11.x1 Mus musculus cDNA, 3 end/clone = IMAGE-1481349/clone_end = 3/gb-AI131744/ | 712.5 | P | 886.2 | P | 733.2 | P | 435.7 |
| | | gi = 3601760/ug = Mm 13773/len = 472/STRA = for | | | | | | | |
| 11 | 103001_at | Cluster Incl U43836 Mus musculus VEGF-related factor mvrf186 precursor mRNA, complete cds/cds = (192,815)/ | 270.6 | P | 325.6 | P | 384.6 | P | 233.7 |
| | | gb = U43836/gi = 1703480/ug = Mm 15607/len = 1236/STRA = for | | | | | | | |
| 11 | 104742_at | Cluster Incl AA919832: vy18b09 r1 Mus musculus cDNA, 5 end/clone = IMAGE-1295801/clone_end = 5/gb = AA919832/ | 339.8 | P | 341.2 | P | 416.3 | P | 196.5 |
| | | gi = 3066611/ug = Mm 24679/len = 474/STRA = for | | | | | | | |
| 11 | 97928_at | Cluster Incl AW045278 UI-M-BH1-akn-c-07-0-UI s1 Mus musculus cDNA, 3 end/clone = UI-M-BH1-akn-c-07-0-UI/ | 171.5 | A | 300.7 | P | 354.7 | P | 117.2 |
| | | clone_end = 3/gb = AW045278/gi = 5905807/ug = Mm 30232/len = 250/STRA = for | | | | | | | |
| 11 | 98004_at | Cluster Incl M63554: Mus musculus inhibitor protein of cAMP-dependent protein kinase, complete cds/cds = (29,259)/ | 78.4 | P | 78.7 | P | 84.2 | P | 50.1 |
| | | gb = M63554/gi = 200384/ug = Mm.3193/len = 2278/STRA = for | | | | | | | |
| 11 | 103755_at | Cluster Incl D89677 Mus musculus mRNA for Kryn, complete cds/cds = (1123,3087)/gb = D89677/gi = 1944454/ | 191.5 | P | 96.9 | P | 365 | P | 78.8 |
| | | ug = Mm 2454/len = 4936/STRA = for | | | | | | | |
| 11 | 104169_at | Cluster Incl D32167 Zinc finger protein of the cerebellum 1/cds = (530,1873)/gb = D32167/gi = 565654/ug = Mm 2719/ | 200.5 | P | 159.4 | P | 140.9 | P | 91.9 |
| | | len = 2947/STRA = for | | | | | | | |
| 11 | 92180_at | Cluster Incl AI851599 UI-M-BH0-ain-a-02-0-UI s1 Mus musculus cDNA, 3 end/clone = UI-M-BH0-ain-a-02-0-UI/ | 275.7 | P | 263.8 | P | 293.2 | P | 179.9 |
| | | clone_end = 3/gb = AI851599/gi = 5495505/ug = Mm 33796/len = 352/STRA = for | | | | | | | |
| 11 | 92322_at | Cluster Incl X94353 Cathelin-like protein/cds = (14,535)/gb = X94353/gi = 1177533/ug = Mm 3834/len = 593/STRA = for | 620.5 | A | 984.1 | P | 888.6 | P | 932.6 |
| 11 | 94801_at | Cluster Incl AI607332 ub76a01 x1 Mus musculus cDNA, 3 end/clone = IMAGE-1383624/clone_end = 3/gb = AI607332/ | 96.9 | P | 113.4 | P | 108.1 | P | 54.8 |
| | | gi = 4616499/ug = Mm 1823/len = 431/STRA = for | | | | | | | |
| 11 | AFFX- | X57349 M musculus mRNA for transferrin receptor (_5, _M, _3 represent transcript regions 5 | 241.3 | P | 329.1 | M | 325.8 | P | 79.8 |
| | TransRecMur/ | prime, Middle, and 3 prime respectively) | | | | | | | |
| | X57349_3_at | | | | | | | | |
| 11 | 93009_at | Cluster Incl J04696 Mouse glutathione S-transferase class mu (GST5-5) mRNA, complete cds/cds = (4,660)/gb = J04696/ | 380.3 | P | 438.8 | P | 620.2 | P | 287.4 |
| | | gi = 193549/ug = Mm 14601/len = 1014/STRA = for | | | | | | | |
| 11 | 94217_f_at | Cluster Incl AC002397 Mouse chromosome 6 BAC-284H12 (Research Genetics mouse BAC library) complete sequence/ | 323 | P | 285.7 | P | 339.8 | P | 242.5 |
| | | cds = (198,998)/gb = AC002397/gi = 3287367/ug = Mm 22228/len = 1155/STRA = for | | | | | | | |
| 11 | 95501_at | Cluster Incl AW122970 UI-M-BH2 1-aoz-h-06-0-UI s2 Mus musculus cDNA, 3 end/clone = UI-M-BH2.1-aoz-h-06-0-UI/ | 215.9 | P | 266.2 | P | 282 | P | 201.9 |
| | | clone_end = 3/gb = AW122970/gi = 6098500/ug = Mm 25157/len = 435/STRA = for | | | | | | | |
| 11 | 99085_at | Cluster Incl AI021421: ub12a08 r1 Mus musculus cDNA, 5 end/clone = IMAGE-1366742/clone_end = 5/gb = AI021421/ | 264 | P | 191.8 | A | 277 | P | 183.3 |
| | | gi = 3235757/ug = Mm 38976/len = 470/STRA = for | | | | | | | |
| 11 | 99111_at | Cluster Incl U09874 Mus musculus SKD3 mRNA, complete cds/cds = (45,2078)/gb = U09874/gi = 563128/ug = Mm 3990/ | 231.8 | P | 256.6 | P | 235.8 | P | 200.6 |
| | | len = 2138/STRA = rev | | | | | | | |
| 11 | 100109_s_at | Cluster Incl X03492 Keratin complex 1, acidic, gene 13/cds = (0,938)/gb = X03492/gi = 52782/ug = Mm 4646/len = 1204/ | 181.9 | P | 231.7 | P | 209.6 | P | 104.3 |
| | | STRA = for | | | | | | | |
| 11 | 102022_at | Cluster Incl AW124555 UI-M-BH2 1-apt-b-05-0-UI s1 Mus musculus cDNA, 3 end/clone = UI-M-BH2 1-apt-b-05-0-UI/ | 218.1 | P | 173.8 | P | 184.1 | P | 139.3 |
| | | clone_end = 3/gb = AW124555/gi = 6100085/ug = Mm.10093/len = 547/STRA = for | | | | | | | |
| 11 | 104135_at | Cluster Incl AW045474 UI-M-BH1-akr-c-02-0-UI s1 Mus musculus cDNA, 3 end/clone = UI-M-BH1-akr-c-02-0-UI/ | 21.4 | P | 94.5 | P | 101 | P | −15.4 |
| | | clone_end = 3/gb = AW045474/gi = 5905968/ug = Mm 22085/len = 318/STRA = for | | | | | | | |
| 11 | 94983_at | Cluster Incl AI851614 UI-M-BH0-ain-b-07-0-UI s1 Mus musculus cDNA, 3 end/clone = UI-M-BH0-ain-b-07-0-UI/ | 209.5 | P | 239.8 | P | 246.9 | P | 222.4 |
| | | clone_end = 3/gb = AI851614/gi = 5495520/ug = Mm 27282/len = 425/STRA = for | | | | | | | |
| 11 | 97995_at | Cluster Incl X61385 Transcription factor 7, T-cell specific/cds = (189,1100)/gb = X61385/gi = 54760/ug = Mm.31630/len = 1790/ | 119.5 | P | 148 | P | 173.2 | P | 123 |
| | | STRA = for | | | | | | | |
| 11 | 102952_g_at | Cluster Incl AJ224738 Mus musculus mRNA for death adaptor molecule (Raidd/Cradd), clone 6-1/cds = (118,717)/ | 127.4 | P | 174.1 | P | 140.9 | P | 119.4 |
| | | gb = AJ224738/gi = 3929835/ug = Mm 17493/len = 1649/STRA = for | | | | | | | |
| 11 | 93431_at | Cluster Incl Z38015 Dystrophia myotonica kinase, B15/cds = (143,2038)/gb = Z38015/gi = 563525/ug = Mm 6529/len = 2755/ | 415 | P | 274 | P | 451.8 | P | 280.7 |
| | | STRA = for | | | | | | | |
| 11 | 93587_at | Cluster Incl L13129 Annexin VII/cds = (42,1433)/gb = L13129/gi = 293293/ug = Mm.20794/len = 2636/STRA = for | 67.9 | P | 180.3 | P | 212.7 | P | 93.2 |
| 11 | 95502_at | Cluster Incl AI840267: UI-M-AH0-act-e-08-0-UI s1 Mus musculus cDNA, 3 end/clone = UI-M-AH0-act-e-08-0-UI/ | 283.9 | P | 189.3 | M | 305.2 | P | 185.6 |
| | | clone_end = 3/gb = AI840267/gi = 5474480/ug = Mm.25161/len = 358/STRA = for | | | | | | | |
| 11 | 97886_at | Cluster Incl AI530375 ui92c09 y1 Mus musculus cDNA, 5 end/clone = IMAGE-1889872/clone_end = 5/gb = AI530375/ | 352 | P | 226.7 | P | 238.8 | P | 235.6 |
| | | gi = 4444510/ug = Mm 28393/len = 474/STRA = for | | | | | | | |
| 11 | 102848_f_at | Cluster Incl AI840577 UI-M-AM0-adp-h-10-0-U1.s1 Mus musculus cDNA, 3 end/clone = UI-M-AM0-adp-h-10-0-UI/ | 239.1 | P | 143.8 | P | 227 | P | 101.2 |
| | | clone_end = 3/gb = AI840577/gi = 5474790/ug = Mm 14808/len = 432/STRA = for | | | | | | | |
| 11 | 94403_at | Cluster Incl AW121330 UI-M-BH2 2-aom-b-01-0-UI s1 Mus musculus cDNA, 3 end/clone = UI-M-BH2 2-aom-b-01-0-UI/ | 209.6 | P | 229.7 | P | 200.6 | A | 280.7 |
| | | clone_end = 3/gb = AW121330/gi = 6096663/ug = Mm 25799/len = 555/STRA = for | | | | | | | |
| 11 | 96240_at | Cluster Incl AI851309 UI-M-BH0-akf-h-03-0-UI s1 Mus musculus cDNA, 3 end/clone = UI-M-BH0-akf-h-03-0-UI/ | 76 | P | 99.1 | P | 155.9 | P | 29.5 |
| | | clone_end = 3/gb = AI851309/gi = 5495215/ug = Mm 28819/len = 207/STRA = for | | | | | | | |
| 11 | 99490_at | Cluster Incl AW050348 UI-M-BH1-ang-d-06-0-UI s1 Mus musculus cDNA, 3 end/clone = UI-M-BH1-ang-d-06-0-UI/ | 127.3 | P | 151.9 | P | 213.9 | P | 165.9 |
| | | clone_end = 3/gb = AW050348/gi = 5910877/ug = Mm 41531/len = 493/STRA = for | | | | | | | |
| 11 | 102824_at | Cluster Incl AF031035: Mus musculus stanniocalcin (Stc2) mRNA, complete cds/cds = (199,1089)/gb = AF031035/ | 161.8 | P | 98.4 | P | 115.6 | P | 164.6 |
| | | gi = 4104012/ug = Mm 12904/len = 1793/STRA = for | | | | | | | |
| 11 | 101350_g_at | Cluster Incl U73170: Polo-like kinase, pseudogene 1/cds = (0,1788)/gb = U73170/gi = 2580507/ug = Mm 57017/len = 1789/ | 450.3 | P | 363.2 | A | 448.8 | A | 294.6 |
| | | STRA = for | | | | | | | |
| 11 | 101182_at | Cluster Incl X95255 GLI-Kruppel family member GLI3/cds = (171,4981)/gb = X95255/gi = 1419013/ug = Mm 5098/len = 5113/ | 59.6 | P | 53.7 | P | 74.9 | P | 43.7 |
| | | STRA = for | | | | | | | |
| 11 | 94518_at | Cluster Incl AW121749 UI-M-BH2 3-aoe-c-12-0-UI s1 Mus musculus cDNA, 3 end/clone = UI-M-BH2 3-aoe-c-12-0-UI/ | 448.1 | P | 506.2 | P | 528 | P | 364.5 |
| | | clone_end = 3/gb = AW121749/gi = 6097084/ug = Mm.28999/len = 313/STRA = for | | | | | | | |
| 11 | 98590_at | Cluster Incl D89571 Mus musculus mRNA for ryudocan core protein, complete cds/cds = (24,620)/gb = D89571/gi = 2373476/ | 67.3 | P | 292.3 | P | 105.6 | A | 62.2 |
| | | ug = Mm 3815/len = 2456/STRA = for | | | | | | | |
| 11 | 100539_at | Cluster Incl AI841279: UI-M-AM0-adu-e-08-0-UI.s1 Mus musculus cDNA, 3 end/clone = UI-M-AM0-adu-e-08-0-UI/ | 322.3 | P | 241.3 | P | 265.6 | P | 177.5 |
| | | clone_end = 3/gb = AI841279/gi = 5475492/ug = Mm.5293/len = 439/STRA = for | | | | | | | |
| 11 | 103671_at | Cluster Incl AF061972 Mus musculus Tat-Interacting protein TIP30 mRNA, complete cds/cds = (191,919)/gb = AF061972/ | 64.6 | A | 67.1 | A | 83.7 | P | 38.9 |
| | | gi = 3820524/ug = Mm 20801/len = 1178/STRA = for | | | | | | | |
| 11 | 95536_at | Cluster Incl AB025015: Mus musculus mRNA for elongin A, complete cds/cds = (8,2329)/gb = AB025015/gi = 6009623/ | 341.1 | P | 299.7 | A | 329.4 | A | 343.4 |
| | | ug = Mm 27663/len = 2537/STRA = for | | | | | | | |
| 11 | 99933_at | Cluster Incl D26107: Mouse mRNA for poliovirus receptor homolog protein soluble form, complete cds/cds = (0,1592)/ | 210.5 | P | 198.4 | P | 147.8 | A | 7.2 |
| | | gb = D26107/gi = 475017/ug = Mm.4341/len = 2443/STRA = for | | | | | | | |
| 11 | 102268_at | Cluster Incl AW123015 UI-M-BH2 1-apb-e-05-0-UI s1 Mus musculus cDNA, 3 end/clone = UI-M-BH2.1-apb-e-05-0-UI/ | 409.4 | A | 427.4 | M | 423.7 | P | 466.5 |
| | | clone_end = 3/gb = AW123015/gi = 6098545/ug = Mm 1116/len = 449/STRA = for | | | | | | | |
| 11 | 102643_at | Cluster Incl M93148 Homeo box A2/cds = (308,1426)/gb = M93148/gi = 193910/ug = Mm 131/len = 1460/STRA = for | 168.8 | P | 186.8 | P | 256.1 | P | 167.8 |
| 11 | 102943_at | Cluster Incl AI099344 ue37d12 y1 Mus musculus cDNA, 5 end/clone = IMAGE-1482551/clone_end = 5/gb = AI099344/ | 84.6 | A | 76.2 | P | 174.3 | P | 98.9 |
| | | gi = 3448869/ug = Mm 17231/len = 364/STRA = for | | | | | | | |
| 11 | 103933_at | Cluster Incl U61183: Yolk sac gene 2/cds = (12,1637)/gb = U61183/gi = 1628564/ug = Mm 2517/len = 2106/STRA = for | −22.4 | P | −32.1 | P | 16 | M | −26 |
| 11 | 102137_f_at | Cluster Incl AI845856 UI-M-AO1-aeg-e-12-0-UI s1 Mus musculus cDNA, 3 end/clone = UI-M-AO1-aeg-e-12-0-UI/ | 878.3 | P | 868.3 | P | 900.4 | P | 712.6 |
| | | clone_end = 3/gb = AI845856/gi = 5489762/ug = Mm 43942/len = 309/STRA = rev | | | | | | | |
| 11 | 95061_at | Cluster Incl AI838996 UI-M-AN0-acm-b-03-0-UI s1 Mus musculus cDNA, 3 end/clone = UI-M-AN0-acm-b-03-0-UI/ | 42.1 | P | 93.9 | P | 86.5 | A | 30.6 |
| | | clone_end = 3/gb = AI638996/gi = 5473209/ug = Mm.29162/len = 417/STRA = for | | | | | | | |
| 11 | 94372_at | Cluster Incl D49956 Mouse mRNA lor 8-oxo-dGTPase, complete cds/cds = (98,568)/gb = D49956/gi = 1065597/ | 359.3 | P | 304.3 | A | 245.8 | A | 151.6 |
| | | ug = Mm.2539/len = 939/STRA = for | | | | | | | |
| 11 | 96833_at | Cluster Incl AW048468 UI-M-BH1-alj-b-03-0-UI s1 Mus musculus cDNA, 3 end/clone = UI-M-BH1-alj-b-03-0-UI/ | 332.9 | P | 266.6 | P | 298.3 | P | 185.2 |
| | | clone_end = 3/gb = AW048468/gi = 5908997/ug = Mm.29420/len = 333/STRA = for | | | | | | | |
| 11 | 101382_at | Cluster Incl AF020198 Mus musculus transcription factor PBX2 (PBX2) mRNA, complete cds/cds = (85,1377)/ | 259.3 | P | 293.1 | P | 350.9 | P | 218.6 |
| | | gb = AF020198/gi = 2432012/ug = Mm 7103/len = 2805/STRA = for | | | | | | | |
| 11 | 101891_at | Cluster Incl Y09517. M musculus mRNA for 17-beta-hydroxysteroid dehydrogenase type II/cds = (94,1239)/gb = Y09517/ | 186.7 | P | 202.6 | A | 177.7 | A | 118.2 |
| | | gi = 2198500/ug = Mm 8877/len = 1342/STRA = for | | | | | | | |
| 11 | 92441_at | Cluster Incl Y10007 Fibroblast activation protein/cds = (170,2455)/gb = Y10007/gi = 1743329/ug = Mm 41816/len = 2715/ | 100.9 | P | 119.4 | P | 110.9 | P | 26.5 |
| | | STRA = for | | | | | | | |
| 11 | 93952_r_at | Cluster Incl AA409629 EST01370 Mus musculus cDNA, 3 end/clone = c0009A09/clone_end = 3/gb = AA409629/ | 9.1 | A | 90.9 | P | 32.2 | P | 4.5 |
| | | gi = 2066285/ug = Mm.94218/len = 305/STRA = rev | | | | | | | |
| 11 | 102094_f_at | Cluster Incl AI841270 UI-M-AM0-adu-d-10-0-UI s1 Mus musculus cDNA, 3 end/clone = UI-M-AM0-adu-d-10-0-UI/ | 578.3 | P | 569.7 | A | 557.9 | A | 383.6 |
| | | clone_end = 3/gb = AI841270/gi = 5475483/ug = Mm 42353/len = 210/STRA = rev | | | | | | | |
| 11 | 101980_at | Cluster Incl J03750 Mouse single stranded DNA binding protein p9 mRNA, complete cds/cds = UNKNOWN/gb = J03750/ | 162.9 | P | 181.8 | P | 166.2 | P | 45.4 |
| | | gi = 192235/ug = Mm.966/len = 2777/STRA = for | | | | | | | |
| 11 | 103607_at | Cluster Incl AI882309 ub98g02 r1 Mus musculus cDNA, 5 end/clone = IMAGE-1396562/clone_end = 5/gb = AI882309/ | 116.4 | A | 170.3 | A | 254.6 | P | 113.1 |
| | | gi = 5567398/ug = Mm.20164/len = 487/STRA = for | | | | | | | |
| 11 | 94406_at | Cluster Incl AJ242864: Mus musculus mRNA 2 for phtf protein/cds = (561,2846)/gb = AJ242864/gi = 6523390/ug = Mm 25877/ | 230.6 | P | 227.3 | A | 86.6 | P | 90.2 |
| | | len = 3330/STRA = for | | | | | | | |
| 11 | 96206_at | Cluster Incl AI854506 UI-M-BH0-ajj-b-09-0-UI s1 Mus musculus cDNA, 3 end/clone = UI-M-BH0-ajj-b-09-0-UI/clone_end = 3/ | 438.4 | A | 538.2 | P | 430.1 | P | 366.9 |
| | | gb = AI854506/gi = 5498412/ug = Mm 28639/len = 413/STRA = for | | | | | | | |
| 11 | 97990_at | Cluster Incl D85923 Myosin heavy chain 11, smooth muscle/cds = (104,6022)/gb = D85923/gi = 1945077/ug = Mm 3153/ | 150.6 | A | 190.8 | M | 266.8 | P | 111.7 |
| | | len = 6175/STRA = rev | | | | | | | |
| 11 | 103708_at | Cluster Incl AI132207: ue28g02.x1 Mus musculus cDNA, 3 end/clone = IMAGE-1481714/clone_end = 3/gb = AI132207/ | 171.4 | P | 53.9 | P | 174.6 | P | 10.7 |
| | | gi = 3602223/ug = Mm 24090/len = 450/STRA = for | | | | | | | |
| 11 | 104395_at | Cluster Incl AW121826 UI-M-BH2.3-anx-e-08-0-UI s2 Mus musculus cDNA, 3 end/clone = UI-M-BH2 3-anx-e-08-0-UI/ | 506.1 | P | 658.9 | A | 586.7 | P | 373.4 |
| | | clone_end = 3/gb = AW121826/gi = 6097196/ug = Mm 28604/len = 413/STRA = for | | | | | | | |
| 11 | 104681_at | Cluster Incl U96963 Diaphanous homolog 1 (Drosophila)/cds = (93,3880)/gb = U96963/gi = 2114472/ug = Mm.3282/len = 4378/ | 143.2 | A | 457.3 | P | 381.6 | A | 231.8 |
| | | STRA = for | | | | | | | |
| 11 | 94140_at | Cluster Incl M59446 Scavenger receptor/cds = (0,20)/gb = M59446/gi = 192739/ug = Mm 1227/len = 413/STRA = for | 141.4 | A | 159 | P | 281.8 | A | 216.2 |
| 11 | 97184_at | Cluster Incl AI528219 uI94h04.y1 Mus musculus cDNA, 5 end/clone = IMAGE-1890103/clone_end = 5/gb = AI528219/ | 181.3 | A | 153.8 | P | 224.8 | P | 148.7 |
| | | gi = 4442354/ug = Mm 36077/len = 505/STRA = for | | | | | | | |
| 11 | 101196_at | Cluster Incl D50060. Proprotein convertase subtilisin/kexin type 6/cds = (0,2798)/gb = D50060/gi = 769700/ug = Mm 5182/ | 62.7 | A | 155.1 | P | 248.4 | P | 61.6 |
| | | len = 3063/STRA = for | | | | | | | |
| 11 | 102120_f_at | Cluster Incl C80148: C80148 Mus musculus cDNA, 3 end/clone = J0077C02/clone_end = 3/gb = C80148/gi = 2520478/ | 969.3 | P | 903.7 | P | 987.1 | M | 667.8 |
| | | ug = Mm 42674/len = 583/STRA = rev | | | | | | | |
| 12 | 92544_f_at | Cluster Incl AF055983. Mus musculus proteasome alpha7/C8 subunit mRNA, complete cds/cds = (42,809)/gb = AF055983/ | 2482.4 | P | 2199.9 | P | 1598.4 | P | 2324.6 |
| | | gi = 3037090/ug = Mm 1007/len = 938/STRA = for | | | | | | | |
| 12 | 92562_at | Cluster Incl U70475: Nuclear, factor, erythroid derived 2, like 2/cds = (139,1932)/gb = U70475/gi = 1778293/ug = Mm 1025/ | 394 | P | 400.7 | P | 174.1 | P | 404.3 |
| | | len = 2380/STRA = for | | | | | | | |
| 12 | 92564_at | Cluster Incl AI891475 uI59a06 x1 Mus musculus cDNA, 3 end/clone = IMAGE-2123314/clone_end = 3/gb = AI891475/ | 199.7 | P | 141.3 | P | 142.6 | P | 199.4 |
| | | gi = 5597377/ug = Mm 10272/len = 493/STRA = for | | | | | | | |
| 12 | 92565_at | Cluster Incl AI847109 UI-M-AP1-agn-g-04-0-UI s1 Mus musculus cDNA, 3 end/clone = UI-M-AP1-agn-g-04-0-UI/ | 624.4 | P | 439.7 | P | 296.1 | P | 427.1 |
| | | clone_end = 3/gb = AI847109/gi = 5491015/ug = Mm 10278/len = 324/STRA = for | | | | | | | |
| 12 | 92569_f_at | Cluster Incl AF053232 Mus musculus SIK similar protein mRNA, complete cds/cds = (270,1691)/gb = AF053232/ | 431.9 | P | 393.1 | P | 218.9 | P | 295.1 |
| | | gi = 2996193/ug = Mm 10303/len = 1879/STRA = for | | | | | | | |
| 12 | 92596_at | Cluster Incl U97327. Mus musculus calcyclin binding protein (CACYBP) mRNA, partial cds/cds = (0,744)/gb = U97327/ | 465.6 | P | 308.6 | P | 157.8 | P | 2551 |
| | | gi = 3138917/ug = Mm. 10702/len = 1404/STRA = for | | | | | | | |
| 12 | 92797_at | Cluster Incl AI840051: UI-M-AL0-abl-a-01-0-UI s1 Mus musculus cDNA, 3 end/clone = UI-M-AL0-abI-a-01-0-UI/ | 623.3 | P | 550.1 | P | 407.5 | P | 518.8 |
| | | clone_end = 3/gb = AI840051/gi = 5474264/ug = Mm 12665/len = 450/STRA = for | | | | | | | |
| 12 | 92831_at | Cluster Incl AI846308: UI-M-AK1-aez-g-10-0-UI s1 Mus musculus cDNA, 3 end/clone = UI-M-AK1-aez-g-10-0-UI/ | 511.8 | P | 608.9 | P | 409.3 | P | 622.1 |
| | | clone_end = 3/gb = AI846308/gi = 5490214/ug = Mm 12983/len = 263/STRA = for | | | | | | | |
| 12 | 93045_at | Cluster Incl L28836: Peroxisomal membrane protein, 70 kDa/cds = (0,1979)/gb = L28836/gi = 454834/ug = Mm.1519/ | 563.2 | P | 543.2 | P | 460.4 | P | 426.6 |
| | | len = 1980/STRA = for | | | | | | | |
| 12 | 93058_at | Cluster Incl AF026481 Mus musculus elF-1A (elF-1A) mRNA, complete cds/cds = (1172,1606)/gb = AF026481/gi = 3746339/ | 496.9 | P | 418.7 | P | 306.9 | P | 431.5 |
| | | ug = Mm 1540/len = 2663/STRA = for | | | | | | | |
| 12 | 93111_at | Cluster Incl D45836 Mouse mRNA for nuclear pore-targeting complex component of 97 kDa, complete cds/cds = (198,2828)/ | 828.6 | P | 741.9 | P | 532 | P | 691.3 |
| | | gb = D45836/gi = 871889/ug = Mm 16710/len = 3897/STRA = for | | | | | | | |
| 12 | 93120_f_at | Cluster Incl V00746 Mus musculus H-2K gene for MHC class I antigen H-2K (allele b), exons 1 to 3 and joined CDS/ | 2809.6 | P | 1919.5 | P | 1473.1 | P | 2679.8 |
| | | cds = (0,1109)/gb = V00746/gi = 51154/ug = Mm 16771/len = 1110/STRA = rev | | | | | | | |
| 12 | 93277_at | Cluster Incl X53584 Mouse mRNA for HSP60 protein (clones 3T3-7, −9, and −M1)/cds = (0,1667)/gb = X53584/gi = 51451/ | 2671.4 | P | 2209.4 | P | 1552.9 | P | 2136.8 |
| | | ug = Mm 1777/len = 2119/STRA = for | | | | | | | |
| 12 | 93310_at | Cluster Incl AI836423 UI-M-AI0-aak-b-03-0-UI s1 Mus musculus cDNA, 3 end/clone = UI-M-AI0-aak-b-03-0-UI/ | 2079.8 | P | 1740 | P | 1153.9 | P | 1507.6 |
| | | clone_end = 3/gb = AI836423/gi = 5470636/ug = Mm 18472/len = 565/STRA = for | | | | | | | |
| 12 | 93547_at | Cluster Incl L03279 Core binding factor beta/cds = UNKNOWN/gb = L03279/gi = 200177/ug = Mm 2018/len = 2685/STRA = for | 2030.9 | P | 1725.1 | P | 1589.4 | P | 1810.6 |
| 12 | 93600_at | Cluster Incl AJ011565 Mus musculus mRNA for leptin receptor gene-related protein/cds = (0,395)/gb = AJ011565 | 729.4 | P | 713.6 | P | 603.7 | P | 670.1 |
| | | gi = 3688429/ug = Mm 20879/len = 635/STRA = for | | | | | | | |
| 12 | 93727_at | Cluster Incl AB003502 Mus musculus mRNA for Guanine Nucleotide Regulatory Protein, partial cds/cds = (0,1652)/ | 611.3 | P | 507.7 | P | 369.9 | P | 589.4 |
| | | gb = AB003502/gi = 3461879/ug = Mm 20925/len = 2079/STRA = for | | | | | | | |
| 12 | 93729_at | Cluster Incl Y15197: Mus musculus mRNA for E-MAP-115 protein/cds = (0,2192)/gb = Y15197/gi = 3417385/ug = Mm 20928/ | 165.6 | P | 112.2 | P | 123.7 | P | 113.2 |
| | | len = 2893/STRA = for | | | | | | | |
| 12 | 93747_at | Cluster Incl AW122599 UI-M-BH2.2-aox-h-11-0-UI s1 Mus musculus cDNA, 3 end/clone = UI-M-BH2 2-aox-h-11-0-UI/ | 1167 | P | 972.7 | P | 784.6 | P | 880.3 |
| | | clone_end = 3/gb = AW122599/gi = 6098125/ug = Mm 21092/len = 456/STRA = for | | | | | | | |
| 12 | 93826_at | Cluster Incl AI956230: ul73a07 y1 Mus musculus cDNA, 5 end/clone = IMAGE-2136180/clone_end = 5/gb = AI956230/ | 967.2 | P | 861.2 | P | 723.4 | P | 982.1 |
| | | gi = 5748967/ug = Mm 21544/len = 513/STRA = for | | | | | | | |
| 12 | 93829_at | Cluster Incl AW107884: um18c08 x 1 Mus musculus cDNA, 3 end/clone = IMAGE-2192654/clone_end = 3/gb = AW107884/ | 1727.3 | P | 1392.5 | P | 1065.2 | P | 1854.4 |
| | | gi = 6078684/ug = Mm 21556/len = 579/STRA = for | | | | | | | |
| 12 | 93832_at | Cluster Incl AI854541: UI-M-BH0-ajj-e-12-0-UI s1 Mus musculus cDNA, 3 end/clone = UI-M-BH0-ajj-o-12-0-UI/clone_end = 3/ | 828.1 | P | 580.5 | P | 245.6 | P | 564.3 |
| | | gb = AI854541/gi = 5498447/ug = Mm 21569/len = 395/STRA = for | | | | | | | |
| 12 | 93970_at | Cluster Incl AI843085: UI-M-AK1-aes-b-10-0-UI s1 Mus musculus cDNA, 3 end/clone = UI-M-AK1-aes-b-10-0-UI/ | 439.4 | P | 398.3 | P | 257.4 | P | 338.8 |
| | | clone_end = 3/gb = AI843085/gi = 5477298/ug = Mm 21659/len = 279/STRA = for | | | | | | | |
| 12 | 93976_at | Cluster Incl AI836686: UI-M-AP0-abh-h-07-0-UI s1 Mus musculus cDNA, 3 end/clone = UI-M-AP0-abh-h-07-0-UI/ | 617.8 | P | 634.8 | P | 561.9 | P | 642 |
| | | clone_end = 3/gb = AI836686/gi = 5470899/ug = Mm 21684/len = 371/STRA = for | | | | | | | |
| 12 | 94008_at | Cluster Incl AW045358: UI-M-BH1-ako-c-05-0-UI s1 Mus musculus cDNA, 3 end/clone = UI-M-BH1-oko-c-05-0-UI/ | 367.3 | P | 251 | P | 214.2 | P | 233.5 |
| | | clone_end = 3/gb = AW045358/gi = 5905887/ug = Mm 21811/len = 296/STRA = for | | | | | | | |
| 12 | 94014_at | Cluster Incl AA760073: vv69b08 r1 Mus musculus cDNA, 5 end/clone = IMAGE-1227639/clone_end = 5/gb = AA760073/ | 828.1 | P | 789.2 | P | 624.2 | P | 827.5 |
| | | gi = 2807867/ug = Mm 2184/len = 404/STRA = for | | | | | | | |
| 12 | 94019_at | Cluster Incl AI852534 UI-M-BH0-alw-e-03-0-UI s1 Mus musculus cDNA, 3 end/clone = UI-M-BH0-aiw-e-03-0-UI/ | 704.4 | P | 708.4 | P | 602 | P | 646.1 |
| | | clone_end = 3/gb = AI852534/gi = 5496440/ug = Mm 21848/len = 265/STRA = for | | | | | | | |
| 12 | 94242_at | Cluster Incl AA881309: vx11d11.r1 Mus musculus cDNA, 5 end/clone = IMAGE-1264149/clone_end = 5/gb = AA881309/ | 1195.7 | P | 1142.8 | P | 978.8 | P | 1248.1 |
| | | gi = 2990619/ug = Mm 22354/len = 461/STRA = for | | | | | | | |
| 12 | 94253_at | Cluster Incl AW061243 UI-M-BH1-amj-c-11-0-UI s2 Mus musculus cDNA, 3 end/clone = UI-M-BH1-amj-c-11-0-UI/ | 834.6 | P | 566.1 | P | 482.6 | P | 663.6 |
| | | clone_end = 3/gb = AW061243/gi = 6008994/ug = Mm 22399/len = 430/STRA = for | | | | | | | |
| 12 | 94281_at | Cluster Incl AW125138 UI-M-BH2 1-apz-c-01-0-UI s1 Mus musculus cDNA, 3 end/clone = UI-M-BH2 1-apz-c-01-0-UI/ | 449.9 | P | 450.5 | P | 352.7 | P | 404.1 |
| | | clone_end = 3/gb = AW125138/gi = 6100668/ug = Mm 22533/len = 352/STRA = for | | | | | | | |
| 12 | 94322_at | Cluster Incl D42048 Mouse mRNA for squalene epoxidase, complete cds/cds = (619,2337)/gb = D42048/gi = 1217592/ | 971.1 | P | 1061.6 | P | 645.7 | P | 1037.5 |
| | | ug = Mm.22663/len = 2611/STRA = for | | | | | | | |
| 12 | 94325_at | Cluster Incl AW124932 UI-M-BH2 1-apu-g-09-0-UI s1 Mus musculus cDNA, 3 end/clone = UI-M-BH2.1-apu-g-09-0-UI/ | 1619.6 | P | 1656.2 | P | 768.9 | P | 1183.9 |
| | | clone_end = 3/gb = AW124932/gi = 6100462/ug = Mm 22675/len = 143/STRA = for | | | | | | | |
| 12 | 94814_at | Cluster Incl AI957190 ul76g02.x1 Mus musculus cDNA, 3 end/clone = IMAGE-2136530/clone_end = 3/gb = AI957190/ | 1564.6 | P | 1334.3 | P | 1108.3 | P | 1290.7 |
| | | gi = 5749899/ug = Mm 22704/len = 499/STRA = for | | | | | | | |
| 12 | 94832_at | Cluster Incl U58105 Mus musculus Btk locus, alpha-D-galactosidase A (Ags), ribosomal protein (L44L), and Bruton a | 280.7 | P | 248.9 | P | 189 | P | 250.9 |
| | | tyrosine kinase (Btk) genes, complete cds/cds = (3,1352)/gb = U58105/gi = 1666699/ug = Mm 22757/len = 2150/STRA = for | | | | | | | |
| 12 | 94865_at | Cluster Incl AW050059 UI-M-BH1-amm-e-04-0-UI s1 Mus musculus cDNA, 3 end/clone = UI-M-BH1-amm-e-04-0-UI/ | 271.8 | P | 198 | P | 198.8 | P | 213.7 |
| | | clone_end = 3/gb = AW050059/gi = 5910588/ug = Mm 23744/len = 335/STRA = for | | | | | | | |
| 12 | 94903_at | Cluster Incl AI850801 UI-M-BG1-aii-f-04-0-UI s1 Mus musculus cDNA, 3 end/clone = UI-M-BG1-aii-f-04-0-UI/clone_end = 3/ | 1241.1 | P | 1190.5 | P | 936.4 | P | 1335.3 |
| | | gb = AI850801/gi = 5494707/ug = Mm 24086/len = 547/STRA = for | | | | | | | |
| 12 | 95408_at | Cluster Incl AW124487: UI-M-BH2 1-ape-h-10-0-UI.s1 Mus musculus cDNA, 3 end/clone = UI-M-BH2.1-ape-h-10-0-UI/ | 1029.3 | P | 985.7 | P | 694.5 | P | 826.1 |
| | | clone_end = 3/gb = AW124487/gi = 6099982/ug = Mm 24220/len = 214/STRA = for | | | | | | | |
| 12 | 95431_at | Cluster Incl AA623426: vn33a01.r1 Mus musculus cDNA, 5 end/clone = IMAGE-1022952/clone_end = 5/gb = AA623426/ | 109.2 | P | 88.9 | P | 84.2 | P | 95.5 |
| | | gi = 2527302/ug = Mm 24463/len = 303/STRA = for | | | | | | | |
| 12 | 95448_at | Cluster Incl AI839371 UI-M-AN0-acj-g-11-0-UI s1 Mus musculus cDNA, 3 end/clone = UI-M-AN0-acj-g-11-0-UI/ | 861 | P | 904.8 | P | 628.5 | P | 923.3 |
| | | clone_end = 3/gb = AI839371/gi = 5473584/ug = Mm 2462/len = 393/STRA = for | | | | | | | |
| 12 | 95467_at | Cluster Incl AW120746 UI-M-BH2.3-any-c-11-0-UI.s1 Mus musculus cDNA, 3 end/clone = UI-M-BH2 3-any-c-11-0-UI/ | 295.3 | P | 272.2 | P | 151.8 | P | 166.7 |
| | | clone_end = 3/gb = AW120746/gi = 6096079/ug = Mm 24763/len = 416/STRA = for | | | | | | | |
| 12 | 95508_at | Cluster Incl X61453: M musculus mRNA for H19 clone/cds = (0,1946)/gb = X61453/gi = 51135/ug = Mm 25203/len = 2092/ | 1662.7 | P | 1300 | P | 1167.6 | P | 1201.5 |
| | | STRA = for | | | | | | | |
| 12 | 96010_at | Cluster Incl AW047023: UI-M-BH1-alp-h-11-0-UI s1 Mus musculus cDNA, 3 end/clone = UI-M-BH1-alp-h-11-0-UI/ | 853.6 | P | 833 | P | 513.5 | P | 550.5 |
| | | clone_end = 3/gb = AW047023/gi = 5907552/ug = Mm 25548/len = 399/STRA = for | | | | | | | |
| 12 | 96011_at | Cluster Incl AB009275 Mus musculus gene for matrin3, complete cds/cds = (0,920)/gb = AB009275/gi = 6497040/ | 310.2 | P | 211.3 | P | 138.7 | P | 159.2 |
| | | ug = Mm.25575/len = 921/STRA = for | | | | | | | |
| 12 | 96012_f_at | Cluster Incl AI835367: UI-M-AQ0-aah-h-02-0-UI.s1 Mus musculus cDNA, 3 end/clone = UI-M-AQ0-aah-h-02-0-UI/ | 2719.6 | P | 2392.9 | P | 1463.8 | P | 1812.9 |
| | | clone_end = 3/gb = AI835367/gi = 5469580/ug = Mm.25575/len = 407/STRA = for | | | | | | | |
| 12 | 96013_l_at | Cluster Incl AI835367: UI-M-AQ0-aah-h-02-0-UI s1 Mus musculus cDNA, 3 end/clone = UI-M-AQ0-aah-h-02-0-UI/ | 363.7 | P | 286.7 | P | 183.1 | P | 229.5 |
| | | clone_end = 3/gb = AI835367/gi = 5469580/ug = Mm.25575/len = 407/STRA = for | | | | | | | |
| 12 | 96019_at | Cluster Incl AI843476: UI-M-AQ1-aec-g-07-0-UI s1 Mus musculus cDNA,3 end/clone = UI-M-AQ1-aoc-g-07-0-UI/ | 1755.3 | P | 1331.6 | P | 1297 | P | 1378.2 |
| | | clone_end = 3/gb = AI843476/gi = 5477889/ug = Mm.256999/len = 339/STRA = for | | | | | | | |
| 12 | 96061_at | Cluster Incl AW060186: UI-M-BH1-amu-e-06-0-UI s1 Mus musculus cDNA, 3 end/clone = UI-M-BH1-amu-e-06-0-UI/ | 372.8 | P | 353.9 | P | 235 | P | 285.1 |
| | | clone_end = 3/gb = AW060186/gi = 6007952/ug = Mm 25699/len = 339/STRA = for | | | | | | | |
| 12 | 96098_at | Cluster Incl AI848416: UI-M-AH1-ags-b-11-0-UI s1 Mus musculus cDNA,3 end/clone = UI-M-AH1-ags-b-11-0-UI/ | 326.5 | P | 311.2 | P | 248.9 | P | 284.2 |
| | | clone_end = 3/gb = AI848416/gi = 5492322/ug = Mm 26775/len = 499/STRA = for | | | | | | | |
| 12 | 96628_at | Cluster Incl X54327: M musculus mRNA for glutamyl-tRNA synthetase/cds = UNKNOWN/gb = X54327/gi = 5804944/ | 414.5 | P | 369.9 | P | 305.1 | P | 348.4 |
| | | ug = Mm 27190/len = 3528/STRA = for | | | | | | | |
| 12 | 96647_at | Cluster Incl AA645440: vq66d12.s1 Mus musculus cDNA, 5 end/clone = IMAGE-1107287/clone_end = 5/gb = AA645440/ | 540.8 | P | 465.4 | P | 446.2 | P | 520.8 |
| | | gi = 2571869/ug = Mm 27293/len = 456/STRA = for | | | | | | | |
| 12 | 96695_at | Cluster Incl AF089812 Mus musculus ubiquitin-conjugating enzyme HR6A mRNA, complete cds/cds = (128,586)/ | 235.2 | P | 182 | P | 151.1 | P | 149.2 |
| | | gb = AF089812/gi = 3769548/ug = Mm 27541/len = 1647/STRA = for | | | | | | | |
| 12 | 97205_at | Cluster Incl AI848056 UI-M-AH1-agp-h-08-0-UI.s1 Mus musculus cDNA, 3 end/clone = UI-M-AH1-agp-h-08-0-UI/ | 928.7 | P | 884.8 | P | 634.5 | P | 775.1 |
| | | clone_end = 3/gb = AI848056/gi = 5491962/ug = Mm 27695/len = 369/STRA = for | | | | | | | |
| 12 | 97237_at | Cluster Incl AI837771 UI-M-AK0-adl-d-01-0-UI s1 Mus musculus cDNA, 3 end/clone = UI-M-AK0-adl-d-01-0-UI/ | 508.8 | P | 428.4 | P | 339.3 | P | 471.3 |
| | | clone_end = 3/gb = AI837771/gi = 5471989/ug = Mm 27831/len = 480/STRA = for | | | | | | | |
| 12 | 97274_at | Cluster Incl Y13071 Mus musculus mRNA for 26S proteasome non-ATPase subunit/cds = (236,1165)/gb = Y13071/ | 1452.1 | P | 1043.4 | P | 905.9 | P | 1247.5 |
| | | gi = 2505939/ug = Mm 27933/len = 1479/STRA = for | | | | | | | |
| 12 | 97292_at | Cluster Incl AI845225 UI-M-BG0-aht-f-07-0-UI.s1 Mus musculus cDNA, 3 end/clone = UI-M-BG0-aht-f-07-0-UI/ | 984.4 | P | 742.5 | P | 486.5 | P | 613.5 |
| | | clone_end = 3/gb = AI845225/gi = 5489131/ug = Mm 28026/len = 258/STRA = for | | | | | | | |
| 12 | 97305_at | Cluster Incl AW123267 UI-M-BH2 1-apg-e-11-0-UI s1 Mus musculus cDNA, 3 end/clone = UI-M-BH2.1-apg-e-11-0-UI/ | 491.1 | P | 402 | P | 365.1 | P | 441.4 |
| | | clone_end = 3/gb = AW123267/gi = 6098797/ug = Mm 28053/len = 147/STRA = for | | | | | | | |
| 12 | 98912_at | Cluster Incl AW047811 UI-M-BH1-als-a-04-0-UI s1 Mus musculus cDNA, 3 end/clone = UI-M-BH1-als-a-04-0-UI/ | 290.1 | P | 311.1 | P | 166.8 | P | 261.8 |
| | | clone_end = 3/gb = AW047811/gi = 5908340/ug = Mm 28600/len = 426/STRA = for | | | | | | | |
| 12 | 98948_at | Cluster Incl AI785289 uj39f05 x1 Mus musculus cDNA, 3 end/clone = IMAGE-1922337/clone_end = 3/gb = AI785289/ | 158 | P | 208.9 | P | 98 | P | 145.2 |
| | | gi = 5333005/ug = Mm 28726/len = 523/STRA = for | | | | | | | |
| 12 | 98956_at | Cluster/Incl AI844979 UI-M-BG0-ahu-c-07-0-UI s1 Mus musculus cDNA, 3 end/clone = UI-M-BG0-ahu-c-07-0-UI/ | 1534.3 | P | 1367.5 | P | 1293.9 | P | 1457.2 |
| | | clone_end = 3/gb = AI844979/gi = 5488885/ug = Mm 28765/len = 245/STRA = for | | | | | | | |
| 12 | 94462_at | Cluster Incl AW120719 UI-M-BH2 3-any-a-06-0-UI s1 Mus musculus cDNA, 3 end/clone = UI-M-BH2 3-any-a-06-0-UI/ | 464.4 | P | 349.1 | P | 319.1 | P | 379.3 |
| | | clone_end = 3/gb = AW120719/gi = 6096052/ug = Mm 28839/len = 482/STRA = for | | | | | | | |
| 12 | 94471 _r_at | Cluster Incl AW045974 UI-M-BH1-alc-a-08-0-UI s1 Mus musculus cDNA, 3 end/clone = UI-M-BH1-alc-a-08-0-UI/ | 122.4 | P | 109.2 | P | 44.6 | P | 91.5 |
| | | clone_end = 3/gb = AW045974/gi = 5906503/ug = Mm 28851/len = 338/STRA = for | | | | | | | |
| 12 | 94488_at | Cluster Incl AI847041 UI-M-AP1-agi-h-03-0-UI s1 Mus musculus cDNA, 3 end/clone = UI-M-AP1-agj-h-03-0-UI/ | 308.5 | P | 267.3 | P | 246.2 | P | 243.9 |
| | | clone_end = 3/gb = AI847041/gi = 5490947/ug = Mm 28899/len = 318/STRA = for | | | | | | | |
| 12 | 95050_at | Cluster Incl AW124547 UI-M-BH2 1-apt-a-04-0-UI.s1 Mus musculus cDNA, 3 end/clone = UI-M-BH2 1-apl-a-04-0-UI/ | 551.5 | P | 420.2 | P | 308.1 | P | 361.1 |
| | | clone_end = 3/gb = AW124547/gi = 6100077/ug = Mm 29138/len = 468/STRA = rev | | | | | | | |
| 12 | 95059_{— at} | Cluster Incl AW047399 UI-M-BH1-ame-g-05-0-UI s1 Mus musculus cDNA, 3 end/clone = UI-M-BH1-ame-g-05-0-UI/ | 1165.8 | P | 864.4 | P | 590.5 | P | 986.6 |
| | | clone_end = 3/gb = AW047399/gi = 5907928/ug = Mm 29159/len = 417/STRA = for | | | | | | | |
| 12 | 95062_at | Cluster Incl AB026997 Mus musculus CAST mRNA for calpastatin, complete cds/cds = (249,2615)/gb = AB026997/ | 499.5 | P | 448.7 | P | 344.8 | P | 448.2 |
| | | gi = 6006274/ug = Mm.29163/len = 2622/STRA = for | | | | | | | |
| 12 | 95158_at | Cluster IncI AF100956 Mus musculus major histocompatibility locus class II region, Fas-binding protein Daxx (DAXX) gene, | 657.1 | P | 694.3 | P | 409.1 | P | 688 |
| | | partial cds: Bing1 (BING1), lapasin (tapasin), RalGDS-like factor (RLF), KE2 (KE2), BING4 (BING4), beta1, 3-galactosyl | | | | | | | |
| | | transferase (be/STRA = for | | | | | | | |
| 12 | 95694_at | Cluster Incl X70956: M musculus T0P gene for topoisomerase I, exons 19-21/cds = (0,347)/gb = X70956/gi = 57932/ | 995.3 | P | 752.5 | P | 454.9 | P | 607.1 |
| | | ug = Mm 29663/len = 348/STRA = for | | | | | | | |
| 12 | 95746_at | Cluster Incl AW123765 UI-M-BH2 1-apm-b-07-0-UI.s1 Mus musculus cDNA, 3 end/clone = UI-M-BH2.1-apm-b-07-0-UI/ | 1392.9 | P | 1247.9 | P | 997.3 | P | 1147.3 |
| | | clone_end = 3/gb = AW123765/gi = 6099295/ug = Mm 29771/len = 402/STRA = for | | | | | | | |
| 12 | 96269_at | Cluster Incl AA716963 vu69f10.r1 Mus musculus cDNA, 5 end/clone = IMAGE-1196883/clone_end = 5/gb = AA716963/ | 638.9 | P | 532.2 | P | 184 | P | 283.8 |
| | | gi = 2729237/ug = Mm 29847/len = 273/STRA = for | | | | | | | |
| 12 | 96277_at | Cluster Incl AI850118 UI-M-BG1-aid-a-10-0-UI.s1 Mus musculus cDNA, 3 end/clone = UI-M-BG1-aid-a-10-0-UI/ | 409.1 | P | 456.9 | P | 290.8 | P | 454.5 |
| | | clone_end = 3/gb = AI850118/gi = 5494024/ug = Mm 29864/len = 311/STRA = for | | | | | | | |
| 12 | 96289_at | Cluster Incl AW061287: UI-M-BH1-anw-b-03-0-UI s1 Mus musculus cDNA, 3 end/clone = UI-M-BH1-anw-b-03-0-UI/ | 596.6 | P | 571.1 | P | 568.2 | P | 636.6 |
| | | clone_end = 3/gb = AW061287/gi = 6009038/ug = Mm 29895/len = 336/STRA = for | | | | | | | |
| 12 | 96331_at | Cluster Incl AI842754: UI-M-AP1-agi-a-10-0-UI.s1 Mus musculus cDNA, 3 end/clone = UI-M-AP1-agi-a-10-0-UI/ | 431.4 | P | 416.4 | P | 276.8 | P | 379.5 |
| | | clone_end = 3/gb = AI842754/gi = 5476967/ug = Mm 29971/len = 325/STRA = for | | | | | | | |
| 12 | 96354_at | Cluster Incl AI854802: UI-M-BH0-aka-f-12-0-UI s1 Mus musculus cDNA, 3 end/clone = UI-M-BH0-aka-f-12-0-UI/ | 1031.4 | P | 734.2 | P | 459.6 | P | 659.1 |
| | | clone_end = 3/gb = AI854802/gi = 5498708/ug = Mm 30007/len = 496/STRA = for | | | | | | | |
| 12 | 96858_at | Cluster Incl AF100927 Mus musculus apoptosis-inducing factor AIF (Aif) mRNA, nuclear gene encoding mitochondrial | 494.1 | P | 407.6 | P | 311 | P | 368.4 |
| | | protein, complele cds/cds = (51,1889)/gb = AF100927/gi = 4323584/ug = Mm 30050/len = 1932/STRA = for | | | | | | | |
| 12 | 96874_g_at | Cluster Incl AI843287: UI-M-AO1-aei-e-06-0-UI.s1 Mus musculus cDNA, 3 end/clone = UI-M-AO1-ael-e-06-0-UI/ | 1614.8 | P | 1464.4 | P | 1224.6 | P | 1370.4 |
| | | clone_end = 3/gb = AI843287/gi = 5477500/ug = Mm 30069/len = 425/STRA = for | | | | | | | |
| 12 | 96878_at | Cluster Incl AW048566: UI-M-BH1-alx-e-09-0-UI s1 Mus musculus cDNA, 3 end/clone = UI-M-BH1-aix-e-09-0-UI/ | 155.1 | P | 128.9 | P | 84.5 | P | 100.3 |
| | | clone_end = 3/gb = AW048566/gi = 5909095/ug = Mm 30073/len = 430/STRA = for | | | | | | | |
| 12 | 96882_at | Cluster Incl AI842936: UI-M-AO1-aem-d-02-0-UI.s1 Mus musculus cDNA, 3 end/clone = UI-M-AO1-aem-d-02-0-UI/ | 2042.6 | P | 1920.8 | P | 1361.6 | P | 1346.3 |
| | | clone_end = 3/gb = AI842936/gi = 5477149/ug = Mm.30078/len = 238/STRA = for | | | | | | | |
| 12 | 96892_at | Cluster Incl AI836804 UI-M-AJ0-abc-h-09-0-UI s1 Mus musculus cDNA, 3 end/clone = UI-M-AJ0-abc-h-09-0-UI | 685.8 | P | 461.3 | P | 365.7 | P | 523.2 |
| | | clone_end = 3/gb = AI836804/gi = 5471017/ug = Mm 30097/len = 512/STRA = for | | | | | | | |
| 12 | 96896_at | Cluster Incl AW214159: uo40f07.x1 Mus musculus cDNA, 3 end/clone = IMAGE-2645029/clone_end = 3/gb = AW214159/ | 1756.6 | P | 1539.9 | P | 1183.5 | P | 1786.5 |
| | | gi = 6520601/ug = Mm.30108/len = 217/STRA = for | | | | | | | |
| 12 | 97471_at | Cluster Incl AI848442 UI-M-AH1-ags-e-10-0-UI s1 Mus musculus cDNA, 3 end/clone = UI-M-AH1-ags-e-10-0-UI/ | 412.2 | P | 405.9 | P | 274.6 | P | 354.3 |
| | | clone_end = 3/gb = AI848442/gi = 5492348/ug = Mm 30547/len = 212/STRA = for | | | | | | | |
| 12 | 97505_at | Cluster Incl AI845999 UI-M-AP1-agi-e-02-0-UI s1 Mus musculus cDNA, 3 end/clone = UI-M-AP1-agi-e-02-0-UI/ | 1081.3 | P | 780.8 | P | 665.5 | P | 813 |
| | | clone_end = 3/gb = AI845999/gi = 5489905/ug = Mm.31435/len = 386/STRA = for | | | | | | | |
| 12 | 97511_at | Cluster Incl AI846600 UI-M-AQ1-aef-e-03-0-UI s1 Mus musculus cDNA, 3 end/clone = UI-M-AQ1-aef-e-03-0-UI/ | 624.5 | P | 758.2 | P | 514.7 | P | 607.4 |
| | | clone_end = 3/gb = AI846600/gi = 5490506/ug = Mm 31616/len = 436/STRA = for | | | | | | | |
| 12 | 97514_at | Cluster Incl AW046438: UI-M-BH1-akp-d-11-0-UI s1 Mus musculus cDNA, 3 end/clone = UI-M-BH1-akp-d-11-0-UI/ | 234 | P | 255.8 | P | 159.5 | P | 208 |
| | | clone_end = 3/gb = AW046438/gi = 5906967/ug = Mm 31946/len = 438/STRA = rev | | | | | | | |
| 12 | 97540_f_at | Cluster Incl M69069 Histocompatibility 2, D region locus 1/cds = (8,1042)/gb = M69069/gi = 199795/ug = Mm.33263/ | 1909.1 | P | 1064.5 | P | 803.7 | P | 1782.9 |
| | | len = 1379/STRA = rev | | | | | | | |
| 12 | 98132_at | Cluster Incl X01756 Cytochrome c, somatic/cds = (0,317)/gb = X01756/gi = 50618/ug = Mm 35389/len = 318/STRA = for | 817.7 | P | 549.6 | P | 397.7 | P | 799.4 |
| 12 | 98136_at | Cluster Incl AF031486 Mus musculus apermidine aminopropyltransferase (Mapmsy) mRNA, complete cds/cds = (76,1176)/ | 502.8 | P | 318.8 | P | 205.6 | P | 498.7 |
| | | gb = AF031486/gi = 2625113/ug = Mm 3553/len = 1663/STRA = for | | | | | | | |
| 12 | 98142_at | Cluster Incl AA930290 vs58d02 r1 Mus musculus cDNA, 5 end/clone = IMAGE-1150467/clone_end = 5/gb = AA930290/ | 287.2 | P | 214.9 | P | 161.3 | P | 218.5 |
| | | gi = 3079883/ug = Mm.35618/len = 285/STRA = for | | | | | | | |
| 12 | 98572_at | Cluster Incl AW122551 UI-M-BH2.2-aox-d-09-0-UI s1 Mus musculus cDNA, 3 end/clone = UI-M-BH2 2-aox-d-09-0-UI/ | 271.7 | P | 198.5 | P | 192.7 | P | 219.3 |
| | | clone_end = 3/gb = AW122551/gi = 6098075/ug = Mm 37516/len = 480/STRA = for | | | | | | | |
| 12 | 98594_at | Cluster Incl AW125453: UI-M-BH2.2-aqm-a-02-0-UI s1 Mus musculus cDNA, 3 end/clone = UI-M-BH2 2-aqm-a-02-0-UI/ | 286.7 | P | 274 | P | 154.3 | P | 181.3 |
| | | clone_end = 3/gb = AW125453/gi = 6100983/ug = Mm.38211/len = 290/STRA = for | | | | | | | |
| 12 | 99107_at | Cluster Incl M31680: Growth hormone receptor/cds = (98,1072)/gb = M31680/gi = 193506/ug = Mm 3986/len = 1150/ | 256.1 | P | 150.3 | P | 180.1 | P | 167 |
| | | STRA = for | | | | | | | |
| 12 | 99138_at | Cluster Incl AA756292 vv40e08 r1 Mus musculus cDNA, 5 end/clone = IMAGE-1224902/clone_end = 5/gb = AA756292/ | 329.9 | P | 285.8 | P | 239.8 | P | 302.9 |
| | | gi = 2803490/ug = Mm.41374/len = 369/STRA = rev | | | | | | | |
| 12 | 99143_at | Cluster Incl AA614914: vo31go1 r1 Mus musculus cDNA, 5 end/clone = IMAGE-1051536/clone_end = 5/gb = AA614914/ | 356 | P | 318.2 | P | 304.6 | P | 346.9 |
| | | gi = 2502142/ug = Mm.4142/len = 375/STRA = for | | | | | | | |
| 12 | 99180_at | Cluster Incl AI851237 UI-M-BH0-akf-a-05-0-UI s1 Mus musculus cDNA, 3 end/clone = UI-M-BH0-akf-a-05-0-UI/ | 854.6 | P | 753.9 | P | 612 | P | 704.2 |
| | | clone_end = 3/gb = AI851237/gi = 5495143/ug = Mm.41800/len = 421/STRA = for | | | | | | | |
| 12 | 99196_at | Cluster Incl AI848532 UI-M-AP1-agf-f-07-0-UI a2 Mus musculus cDNA, 3 end/clone = UI-M-AP1-agf-07-0-UI/ | 1456.4 | P | 872.6 | P | 690.6 | P | 1064.8 |
| | | clone_end = 3/gb = AI848532/gi = 5492438/ug = Mm 41939/len = 378/STRA = for | | | | | | | |
| 12 | 99579_at | Cluster Incl U59761: ATPase, Na+/K+beta 3 polypeptide/cds = (148,984)/gb = U59761/gi = 1762431/ug = Mm 424/len = 1842/ | 2417 | P | 2046 | P | 1720.8 | P | 2562.5 |
| | | STRA = for | | | | | | | |
| 12 | 100088_at | Cluster Incl M27073 Protein phosphatase 1, catalytic sutunit, beta isoform/cds = (95,1078)/gb = M27073/gi = 471977/ | 1415.3 | P | 1408.2 | P | 966.6 | P | 1121.3 |
| | | ug = Mm 4572/len = 2243/STRA = for | | | | | | | |
| 12 | 100144_at | Cluster Incl X07699 Nucleolin/cds = (0,2123)/gb = X07699/gi = 53453/ug = Mm 4907/len = 2124/STRA = for | 3477.8 | P | 3315.1 | P | 1950.4 | P | 3296.2 |
| 12 | 100560_at | Cluster Incl U95116 Platelet-activating factor acetylhydrolase, isoform 1b, beta 1 subunit/cds = (859,2091)/gb = U95116/ | 422.5 | P | 314.6 | P | 268.8 | P | 267 |
| | | gi = 2072302/ug = Mm.56337/len = 5832/STRA = for | | | | | | | |
| 12 | 100568_at | Cluster Incl U90446: Mus musculus RNAse L Inhibitor (Mu-RLI) mRNA, complete cds/cds = (114,1913)/gb = U90446/ | 949.5 | P | 743.8 | P | 502.2 | P | 566.1 |
| | | gi = 3273416/ug = Mm.5831/len.= 3509/STRA = for | | | | | | | |
| 12 | 101019_at | Cluster Incl U74683 Cathepsin C/cds = (67,1455)/gb = U74683/gi = 2145015/ug = Mm 684/len = 1767/STRA = for | 1304.4 | P | 1121.6 | P | 1047.9 | P | 1298.5 |
| 12 | 101023_f_at | Cluster Incl AI843605 UI-M-AO1-aeh-h-06-0-UI s1 Mus musculus cDNA, 3 end/clone = UI-M-AO1-aeh-h-06-0-UI/ | 951.5 | P | 1277.1 | P | 766.6 | P | 1093.3 |
| | | clone_end = 3/gb = AI843605/gi = 5477783/ug = Mm.6844/len = 379/STRA = for | | | | | | | |
| 12 | 101035_at | Cluster Incl U35846 Mus musculus mRNA, complete cds/cds = (85,1599)/gb = U35846/gi = 2623754/ug = Mm 692/len = 3715/ | 474.9 | P | 468.1 | P | 321.4 | P | 330.8 |
| | | STRA = for | | | | | | | |
| 12 | 101057_at | Cluster Incl AW121180 UI-M-BH2 3-aok-a-08-0-UI s1 Mus musculus cDNA, 3 end/clone = UI-M-BH2 3-aok-a-08-0-UI/ | 438.9 | P | 396.4 | P | 272.7 | P | 424.9 |
| | | clone_end = 3/gb = AW121180/gi = 6096513/ug = Mm 7066/len = 437/STRA = for | | | | | | | |
| 12 | 101094_at | Cluster Incl AI836820 UI-M-AJ0-abd-c-01-0-UI s1 Mus musculus cDNA, 3 end/clone = UI-M-AJ0-abd-c-01-0-UI/ | 497.1 | P | 549.6 | P | 430.1 | P | 541 |
| | | clone_end = 3/gb = AI836820/gi = 5471033/ug = Mm.7381/1en = 357/STRA = for | | | | | | | |
| 12 | 101097_at | Cluster Incl AW121176 UI-M-BH2 3-aok-a-03-0-UI.s1 Mus musculus cDNA, 3 end/clone = UI-M-BH2 3-aok-a-03-0-UI/ | 379.7 | P | 267.7 | P | 207.3 | P | 281.8 |
| | | clone_end = 3/gb = AW121176/gi = 6096509/ug = Mm.74084/len = 435/STRA = for | | | | | | | |
| 12 | 101476_at | Cluster Incl U93050 Mus musculus poly(A) binding protein II (mPABII) gene, complete cds/cds = (0,908)/gb = U93050/ | 1016.8 | P | 1081.9 | P | 636.6 | P | 1058.6 |
| | | gi = 2351845/ug = Mm 7723/len = 909/STRA = for | | | | | | | |
| 12 | 101489_at | Cluster Incl D12780 Mouse mRNA for S-adenosylmethionine decarboxylase, complete cds/cds = (326,1330)/gb = D12780/ | 613.6 | P | 721.2 | P | 323.4 | P | 627.1 |
| | | gi = 220330/ug = Mm 7880/len = 1497/STRA = for | | | | | | | |
| 12 | 101518_at | Cluster Incl U38981 Mus musculus uterine mRNA, complete cds/cds = (205,603)/gb = U38981/gi = 1066284/ug = Mm 8486/ | 293.9 | P | 251.7 | P | 193 | P | 225.5 |
| | | len = 651/STRA = for | | | | | | | |
| 12 | 101523_at | Cluster Incl AW046194: UI-M-BH1-akw-f-03-0-UI.s1 Mus musculus cDNA, 3 end/clone-UI-M-BH1-akw-f-03-0-UI/ | 3682.1 | P | 3128.6 | P | 1945.3 | P | 2982.5 |
| | | clone_end = 3/gb = AW046194/gi = 5906723/ug = Mm 86187/len = 380/STRA = for | | | | | | | |
| 12 | 101527_at | Cluster Incl M18209 Mouse transcription factor S-II, clone PSII-2/cds = (121,921)/gb = M18209/gi = 201936/ug = Mm 87235/ | 880.7 | P | 828.1 | P | 870.3 | P | 876.7 |
| | | len = 2466/STRA = for | | | | | | | |
| 12 | 101528_at | Cluster Incl D00925 Mus musculus mRNA for transcription factor S-II-related proteins, complete cds/cds = UNKNOWN/ | 459.3 | P | 457.7 | P | 389.8 | P | 405.9 |
| | | gb = D00925/gi = 220590/ug = Mm.87235/len = 2257/STRA = for | | | | | | | |
| 12 | 101529_g_at | Cluster Incl D00925 Mus musculus mRNA for transcription factor S-II-related proteins, complete cds/cds = UNKNOWN/ | 465.3 | P | 279.7 | P | 292.2 | P | 354 |
| | | gb = D00925/gi = 220590/ug = Mm.87235/len = 2257/STRA = for | | | | | | | |
| 12 | 101548_at | Cluster Incl AI846345: UI-M-AN1-afa-c-08-0-UI s1 Mus musculus cDNA, 3 end/clone = UI-M-AN1-afa-c-08-0-UI/ | 183.6 | P | 163.7 | P | 105.4 | P | 152.7 |
| | | clone_end = 3/gb = AI846345/gi = 5490251/ug = Mm 88572/len = 394/STRA = rev | | | | | | | |
| 12 | 101955_at | Cluster Incl AJ002387 Mus musculus mRNA for BiP/cds = (11,1978)/gb = AJ002387/gi = 2598561/ug = Mm 918/len = 2282/ | 1926.3 | P | 1399.5 | P | 750.6 | P | 1789 |
| | | STRA = for | | | | | | | |
| 12 | 102317_at | Cluster Incl AF061516 Mus musculus vesicle associated membrane protein 4 (Vamp4) mRNA, complete cds/cds = (77,502)/ | 348 | P | 340 | P | 342.4 | P | 373.7 |
| | | gb = AF061516/gi = 3108178/ug = Mm 10699/len = 819/STRA = for | | | | | | | |
| 12 | 102786_at | Cluster Incl AI849432: UI-M-AJ1-ahc-f-04-0-UI.s1 Mus musculus cDNA, 3 end/clone = UI-M-AJ1-ahc-f-04-0-UI/ | 314.2 | P | 294.6 | P | 179.8 | P | 249.6 |
| | | clone_end = 3/gb = AI849432/gi = 5493338/ug = Mm.13796/len = 291/STRA = for | | | | | | | |
| 12 | 103021_r_at | Cluster Incl AI317205: ui35g09 y1 Mus musculus cDNA 5 end/clone = IMAGE-1853344/clone_end = 5/gb = AI317205/ | 412.6 | P | 328.2 | P | 185.4 | P | 337.7 |
| | | gi = 4032472/ug = Mm 15918/len = 235/STRA = for | | | | | | | |
| 12 | 103056_at | Cluster Incl AI849721 UI-M-AL1-ahn-b-07-0-UI s1 Mus musculus cDNA, 3 end/clone = UI-M-AL1-ahn-b-07-0-UI/ | 305.9 | P | 343.8 | P | 137.7 | P | 341 |
| | | clone_end = 3/gb = AI849721/gi = 5493627/ug = Mm 1654/len = 352/STRA = for | | | | | | | |
| 12 | 103064_at | Cluster Incl AF016583: Mus musculus checkpoint kinase Chk1 (Chk1) mRNA, complete cds/cds = (22,1452)/gb = AF016583/ | 286 | P | 205.7 | P | 163.8 | P | 153.3 |
| | | gi = 2367670/ug = Mm 16753/len = 1962/STRA = for | | | | | | | |
| 12 | 103074_f_at | Cluster Incl AI842969 UI-M-AO1-aem-g-05-0-UI s1 Mus musculus cDNA, 3 end/clone = UI-M-AO1-aem-g-05-0-UI/ | 482.1 | P | 491.2 | P | 453.7 | P | 440.2 |
| | | clone_end = 3/gb = AI842969/gi = 5477182/ug = Mm 16976/len = 340/STRA = for | | | | | | | |
| 12 | 103086_at | Cluster Incl Y00208: Homeo box A5/cds = (74,886)/gb = Y00208/gi = 51371/ug = Mm 173/len = 1678/STRA = for | 249.3 | P | 154.6 | P | 134.4 | P | 183.4 |
| 12 | 103370_at | Cluster Incl AI848387 UI-M-AH1-ago-e-07-0-UI.s1 Mus musculus cDNA, 3 end/clone = UI-M-AH1-ago-e-07-0-UI/ | 1283.8 | P | 959.8 | P | 721.2 | P | 736.9 |
| | | clone_end = 3/gb = AI848367/gi = 5492268/ug = Mm 18555/len = 533/STRA = for | | | | | | | |
| 12 | 103400_at | Cluster Incl D50523 Mouse mRNA for TI-227/cds = UNKNOWN/gb = D50523/gi = 1166394/ug = Mm 18728/len = 3577/ | 598.5 | P | 525 | P | 406.6 | P | 456.3 |
| | | STRA = for | | | | | | | |
| 12 | 103654_at | Cluster Incl AB018374 Mus musculus GARP45 mRNA, complete cds/cds = (69,1289)/gb = AB018374/gi = 5263197/ | 522.9 | P | 269 | P | 260.4 | P | 214.2 |
| | | ug = Mm 20450/len = 1865/STRA = for | | | | | | | |
| 12 | 103657_i_at | Cluster Incl AI836552 UI-M-AP0-abe-e-08-0-UI.s1 Mus musculus cDNA, 3 end/clone = UI-M-AP0-abe-e-08-0-UI/ | 305.6 | P | 266.6 | P | 103.9 | P | 179.3 |
| | | clone_end = 3/gb = AI836552/gi = 5470765/ug = Mm.20559/len = 417/STRA = for | | | | | | | |
| 12 | 103874_r_at | Cluster Incl AA684456 vm64c06 s1 Mus musculus cDNA, 5 end/clone = IMAGE-1003018/clone_end = 5/gb = AA684456/ | 103.3 | P | 72.8 | P | 57.4 | P | 84.2 |
| | | gi = 2671042/ug = Mm.21173/len = 217/STRA = for | | | | | | | |
| 12 | 103892_r_at | Cluster Incl AI197161 ue51h10 r1 Mus musculus cDNA, 5 end/clone = IMAGE-1494691/clone_end = 5/gb = AI197161/ | 304.1 | P | 245.3 | P | 203.2 | P | 255.2 |
| | | gi = 3749767/ug = Mm 21288/len = 475/STRA = for | | | | | | | |
| 12 | 104046_at | Cluster Incl AI854141 UI-M-BH0-ajw-f-12-0-UI s1 Mus musculus cDNA, 3 end/clone = UI-M-BH0-ajw-f-12-0-UI/ | 753.9 | P | 597.1 | P | 613.4 | P | 565.4 |
| | | clone_end = 3/gb = AI854141/gi = 5498047/ug = Mm.21489/len = 577/STRA = for | | | | | | | |
| 12 | 104080_at | Cluster Incl AW212479 uo89c10 x1 Mus musculus cDNA, 3 end/clone = IMAGE-2649714/clone_end = 3/gb = AW212479/ | 831.8 | P | 762 | P | 734.9 | P | 868.9 |
| | | gi = 6518566/ug = Mm 21753/len = 163/STRA = for | | | | | | | |
| 12 | 104115_at | Cluster Incl AA867340: vx97a11 r1 Mus musculus cDNA, 5 end/clone = IMAGE-1293884/clone_end = 5/gb = AA867340/ | 518.1 | P | 525.2 | P | 312.6 | P | 466.3 |
| | | gi = 2962785/ug = Mm.21963/len = 308/STRA = for | | | | | | | |
| 12 | 104303_i_at | Cluster Incl AW124151 UI-M-BH2 1-apr-f-08-0-UI s1 Mus musculus cDNA, 3 end/clone = UI-M-BH2.1-apr-f-08-0-UI/ | 194.6 | P | 150 | P | 74.6 | P | 157.4 |
| | | clone_end = 3/gb = AW124151/gi = 6099681/ug = Mm.22358/len = 298/STRA = for | | | | | | | |
| 12 | 104304_r_at | Cluster Incl AW124151 UI-M-BH2 1-apr-f-08-0-UI s1 Mus musculus cDNA, 3 end/clone = UI-M-BH2.1-apr-f-08-0-UI/ | 235.1 | P | 237.8 | P | 63.1 | P | 174.9 |
| | | clone_end = 3/gb = AW124151/gi = 6099681/ug = Mm.22358/len = 298/STRA = for | | | | | | | |
| 12 | 104531_at | Cluster Incl X60304 Protein kinase C, delta/cds = (13,2037)/gb = X60304/gi = 53436/ug = Mm 2314/len = 2538/STRA = rev | 1308.5 | P | 816 | P | 885.5 | P | 929.6 |
| 12 | 104567_at | Cluster Incl AI848526 UI-M-AP1-agf-e-11-0-UI s2 Mus musculus cDNA, 3 end/clone = UI-M-AP1-agf-e-11-0-UI/ | 168 | P | 120.2 | P | 93.8 | P | 107.9 |
| | | clone_end = 3/gb = AI848526/gi = 5492432/ug = Mm 23880/len = 423/STRA = for | | | | | | | |
| 12 | 104578_f_at | Cluster Incl AI195392 ue72102 r1 Mus musculus cDNA, 5 end/clone = IMAGE-1496667/clone_end = 5/gb = AI195392/ | 1059.3 | P | 1095.7 | P | 727.4 | P | 1290.6 |
| | | gi = 3747998/ug = Mm 23961/len = 262/STRA = for | | | | | | | |
| 12 | 104595_at | Cluster Incl AI846890 UI-M-AK1-aey-a-05-0-UI s1 Mus musculus cDNA, 3 end/clone = UI-M-AK1-aey-a-05-0-UI/ | 423.5 | P | 317.8 | P | 161.6 | P | 195.9 |
| | | clone_end = 3/gb = AI846890/gi = 5490796/ug = Mm.24025/len = 346/STRA = rev | | | | | | | |
| 12 | 104738_at | Cluster Incl D63784 Mouse mRNA for MIDA1, complete cds/cds = (112,1977)/gb = D63784/gi = 1060924/ug = Mm 2467/ | 345.6 | P | 429.5 | P | 290.6 | P | 438.7 |
| | | len = 2037/STRA = for | | | | | | | |
| 12 | 104745_at | Cluster Incl AA763874 vv48a12.r1 Mus musculus cDNA, 5 end/clone = IMAGE-1225630/clone_end = 5/gb = AA763874/ | 381.3 | P | 318.4 | P | 135.8 | P | 170.3 |
| | | gi = 2811396/ug = Mm.24719/len = 443/STRA = for | | | | | | | |
| 12 | 104749_at | Cluster Incl AW123623 UI-M-BH2.1-api-e-02-0-UI s1 Mus musculus cDNA, 3 end/clone = UI-M-BH2.1-api-e-02-0-UI/ | 212.5 | P | 178.9 | P | 137.9 | P | 184.2 |
| | | clone_end = 3/gb = AW123623/gi = 6099153/ug = Mm 24765/len = 224/STRA = for | | | | | | | |
| 12 | 94343_at | Cluster Incl AI604013 vr15c03 y1 Mus musculus cDNA, 5 end/clone = IMAGE-1120708/clone_end = 5/gb = AI604013/ | 245.6 | P | 219.4 | P | 183 | P | 237.9 |
| | | gi = 4613175/ug = Mm.25122/len = 164/STRA = for | | | | | | | |
| 12 | 94361_at | Cluster Incl AI836083 UI-M-AJ0-abb-f-06-0-UI s1 Mus musculus cDNA, 3 end/clone = UI-M-AJ0-abb-f-06-0-UI/ | 575.3 | P | 742.2 | P | 469 | P | 587 |
| | | clone_end = 3/gb = AI836083/gi = 5470296/ug = Mm.25264/len = 529/STRA = for | | | | | | | |
| 12 | 94985_at | Cluster Incl AI846392 UI-M-AN1-afa-h-02-0-UI s1 Mus musculus cDNA, 3 end/clone = UI-M-AN1-afa-h-02-0-UI/ | 389.1 | P | 514 | P | 254.7 | P | 371 |
| | | clone_end = 3/gb = AI846392/gi = 5490298/ug = Mm.27303/len = 399/STRA = for | | | | | | | |
| 12 | 95522_i_at | Cluster Incl AB024005.Mus musculus mRNA for KRAB-containing zinc-finger protein KRAZ2, complete cds/ | 378.6 | P | 325.3 | P | 192 | P | 295.6 |
| | | cds = (307,2127)/gb = AB024005/gi = 4514560/ug = Mm.27575/len = 2247/STRA = for | | | | | | | |
| 12 | 95530_at | Cluster Incl AW060250 UI-M-BH1-amv-e-11-0-UI s1 Mus musculus cDNA, 3 end/clone = UI-M-BH1-amv-e-11-0-UI/ | 276.6 | P | 265 | P | 177.5 | P | 225.3 |
| | | clone_end = 3/gb = AW060250/gi = 6008041/ug = Mm.27629/len = 581/STRA = for | | | | | | | |
| 12 | 95594_at | Cluster Incl AI847486 UI-M-AI1-afs-g-07-0-UI.s1 Mus musculus cDNA, 3 end/clone = UI-M-AI1-afs-g-07-0-UI/clone_end = 3/ | 413 | P | 407.9 | P | 387.8 | P | 365.7 |
| | | gb = AI847486/gi = 5491392/ug = Mm 27914/len = 349/STRA = for | | | | | | | |
| 12 | 95611_at | Cluster Incl AA726364: vu90e02 r1 Mus musculus cDNA, 5 end/clone = IMAGE-1209434/clone_end = 5/gb = AA726364/ | 1517.6 | P | 1849.4 | P | 1158.3 | P | 1559 |
| | | gi = 2744071/ug = Mm.27994/len = 374/STRA = for | | | | | | | |
| 12 | 95613_at | Cluster Incl AW122573 UI-M-BH2 2-aox-f-07-0-UI s1 Mus musculus cDNA, 3 end/clone = UI-M-BH2 2-aox-f-07-0-UI/ | 452.8 | P | 352.2 | P | 280.5 | P | 383.9 |
| | | clone_end = 3/gb = AW122573/gi = 6098098/ug = Mm.28001/len = 448/STRA = rev | | | | | | | |
| 12 | 96135_at | Cluster Incl AA833425: ub58d03 r1 Mus musculus cDNA, 5 end/clone = IMAGE-1381925/clone_end = 5/gb = AA833425/ | 1192.4 | P | 1251.8 | P | 932.5 | P | 1034 |
| | | gi = 2907153/ug = Mm.28149/len = 490/STRA = for | | | | | | | |
| 12 | 96152_at | Cluster Incl AI645561: vt98h08 x1 Mus musculus cDNA, 3 end/clone = IMAGE-1179231/clone_end = 3/gb = AI645561/ | 620.6 | P | 392.9 | P | 207.2 | P | 414.8 |
| | | gi = 4724036/ug = Mm 28256/len = 332/STRA = for | | | | | | | |
| 12 | 96755_at | Cluster Incl AA638002: vr30d10 r1 Mus musculus cDNA, 5 end/clone = IMAGE-1122163/clone_end = 5/gb = AA638002/ | 839.4 | P | 856 | P | 783.1 | P | 792.1 |
| | | gi = 2561590/ug = Mm 28982/len = 136/STRA = for | | | | | | | |
| 12 | 96773_at | Cluster Incl AW125408: UI-M-BH2 3-aqh-d-12-0-UI s1 Mus musculus cDNA, 3 end/clone = UI-M-BH2 3-aqh-d-12-0-UI/ | 337.4 | P | 260.9 | P | 267.5 | P | 331 |
| | | clone_end = 3/gb = AW125408/gi = 6100938/ug = Mm 29036/len = 480/STRA = for | | | | | | | |
| 12 | 96836_r_at | Cluster Incl AI447619: mr29c12.x1 Mus musculus cDNA, 3 end/clone = IMAGE-598870/clone_end = 3/gb = AI447619/ | 52.2 | P | 46 | P | 35.2 | P | 46.7 |
| | | gi = 4305257/ug = Mm.29434/len = 217/STRA = for | | | | | | | |
| 12 | 97394_at | Cluster Incl AA794509: vr46h05 s1 Mus musculus cDNA, 5 end/clone = IMAGE-1123737/clone_end = 5/gb = AA794509/ | 353.8 | P | 292.4 | P | 169.8 | P | 207.3 |
| | | gi = 2857464/ug= Mm 29825/len = 571/STRA = for | | | | | | | |
| 12 | 97947_at | Cluster Incl AI836959: UI-M-AJ0-aaz-b-08-0-UI s1 Mus musculus cDNA, 3 end/clone = UI-M-AJ0-aaz-b-08-0-UI/ | 474.2 | P | 458.4 | P | 393.9 | P | 490.2 |
| | | clone_end = 3/gb = AI836959/gi = 5471172/ug = Mm 3037/len = 449/STRA = for | | | | | | | |
| 12 | 97991_at | Cluster Incl X02452: Mouse Ki-ras cellular oncogene exon 1 from Y1 adrenal tumor cells (and joined CDS)/cds = (11,577)/ | 328.2 | P | 266.2 | P | 226.3 | P | 239.4 |
| | | gb = X02452/gi = 52798/ug= Mm 31530/len = 1722/STRA = for | | | | | | | |
| 12 | 98013_at | Cluster Incl AA666464: vm48a05.r1 Mus musculus cDNA, 5 end/clone = IMAGE-1001456/clone_end = 5/gb = AA666464/ | 278.7 | P | 220.7 | P | 231.6 | P | 229.3 |
| | | gi = 2625165/ug = Mm 3224/len = 164/STRA = for | | | | | | | |
| 12 | 98438_f_at | Cluster Incl X16202 Mouse Q4 class 1 MHC gene (exon 5)/cds = (0,91)/gb = X16202/gi = 53862/ug = Mm 34421/len = 116/ | 1906.5 | P | 1008.5 | P | 806.4 | P | 1335.7 |
| | | STRA = for | | | | | | | |
| 12 | 98441_at | Cluster Incl L23971 Fragile X mental retardation syndrome 1 homolog/cds = (123,1967)/gb = L23971/gi = 398044/ | 402.4 | P | 338.6 | P | 158.5 | P | 251.2 |
| | | ug = Mm.3451/len = 4240/STRA = rev | | | | | | | |
| 12 | 98491_at | Cluster Incl AI286904: ui78d09 y1 Mus musculus cDNA, 5 end/clone = IMAGE-1888529/clone_end = 5/gb = AI286904/ | 303.4 | P | 238.1 | P | 281.3 | P | 279.6 |
| | | gi = 3926657/ug_Mm 35596/len = 352/STRA = for | | | | | | | |
| 12 | 98509_at | Cluster Incl AI834979 UI-M-AM1-afw-e-03-0-UI s1 Mus musculus cDNA, 3 end/clone = UI-M-AM1-afw-e-03-0-UI/ | 404.7 | P | 345.7 | P | 286.8 | P | 387.5 |
| | | clone_end = 3/gb = AI834979/gi = 5469192/ug = Mm.3584/len = 451/STRA = for | | | | | | | |
| 12 | 99462_at | Cluster Incl D38046 Mouse mRNA for typeII DNA topoisomerase beta isoform, complete cds/cds = (46,4884)/gb = D38046/ | 465.6 | P | 377.4 | P | 187.3 | P | 248.6 |
| | | gi = 533330/ug = Mm.4093/len = 5207/STRA = for | | | | | | | |
| 12 | 99467_at | Cluster Incl AI846922.UI-M-AK1-aey-d-05-0-UI s1 Mus musculus cDNA, 3 end/clone = UI-M-AK1-aey-d-05-0-UI/ | 461.4 | P | 383.8 | P | 175 | P | 269.2 |
| | | clone_end = 3/gb = AI846922/gi = 5490828/ug = Mm 41224/len = 381/STRA = for | | | | | | | |
| 12 | 99935_at | Cluster Incl D14340 Tight junction protein 1/cds = 403,5640)/gb = D14340/gi = 303709/ug = Mm 4342/len = 7046/STRA = for | 516.3 | P | 355.9 | P | 315.5 | P | 288.7 |
| 12 | 99949_at | Cluster Incl AW060324 UI-M-BH1-anh-a-06-0-UI s1 Mus musculus cDNA, 3 end/clone = UI-M-BH1-anh-a-06-0-UI/ | 158.7 | P | 173.1 | P | 63.5 | P | 179.2 |
| | | clone_end = 3/gb = AW060324/gi = 6008075/ug = Mm 43681/len = 475/STRA = for | | | | | | | |
| 12 | 99959_at | Cluster Incl AW061337 UI-M-BH1-anw-f-11-0-UI.s1 Mus musculus cDNA, 3 end/clone = UI-M-BH1-anw-f-11-0-UI/ | 124 | P | 117.7 | P | 74.9 | P | 125.5 |
| | | clone_end = 3/gb = AW061337/gi = 6009088/ug = Mm 4410/len = 475/STRA = for | | | | | | | |
| 12 | 99985_at | Cluster Incl AB027565 Mus musculus TXNRD1 mRNA for thioredoxin reductase 1, complete cds/cds = (158,1657)/ | 699.2 | P | 519.6 | P | 467.9 | P | 615.4 |
| | | gb = AB027565/gi = 6467192/ug= Mm 44552/len = 3328/STRA = for | | | | | | | |
| 12 | 99990_at | Cluster Incl U28789 Mus musculus p53-associated cellular protein PACT mRNA, partial cds/cds = (0,4764)/gb = U28789/ | 625 | P | 500.1 | P | 229.4 | P | 363.5 |
| | | gi = 1546778/ug = Mm 4480/len = 5191/STRA = for | | | | | | | |
| 12 | 100400_at | Cluster Incl AW124329.UI-M-BH2 1-apq-e-12-0-UI s1 Mus musculus cDNA, 3 end/clone = UI-M-BH2 1-apq-e-12-0-UI/ | 140.9 | P | 124.4 | P | 83.5 | P | 118.9 |
| | | clone_end = 3/gb = AW124329/gi = 6099859/ug = Mm 464/len = 236/STRA = for | | | | | | | |
| 12 | 100464_at | Cluster Incl AI840585: UI-M-AM0-adq-a-06-0-UI s1 Mus musculus cDNA, 3 end/clone = UI-M-AM0-adq-a-06-0-UI/ | 466.2 | P | 282.1 | P | 226.6 | P | 376.5 |
| | | clone_end = 3/gb = AI840585/gi = 5474798/ug = Mm 491/len = 447/STRA = for | | | | | | | |
| 12 | 100469_at | Cluster Incl D78642 Nuclear transcription factor-Y alpha/cds = UNKNOWN/gb = D78642/gi = 1304151/ug = Mm 4929/ | 224.7 | P | 179.8 | P | 167.6 | P | 183.4 |
| | | len = 2121/STRA = for | | | | | | | |
| 12 | 100954_at | Cluster Incl AF057287 Mus musculus RAB/Rip protein mRNA, partial cds/cds = (0,983)/gb = AF057287/gi = 3063650/ | 253.5 | P | 181.8 | P | 212.8 | P | 242.6 |
| | | ug = Mm 6461/len = 1598/STRA = for | | | | | | | |
| 12 | 100980_at | Cluster Incl U58512 Mus musculus Rho-associated, coiled-coil forming protein kinase p160 ROCK-1 mRNA, complete cds/ | 271.9 | P | 270.9 | P | 178.5 | P | 188.6 |
| | | cds = (0,4064)/gb = U58512/gi = 1514695/ug = Mm 6710/len = 4065/STRA = for | | | | | | | |
| 12 | 101459_at | Cluster Incl L10410 Chromodomain helicase DNA binding protein 1/cds = (170,5305)/gb = L10410/gi = 455014/ug = Mm 8137/ | 315.1 | P | 278.2 | P | 222.6 | P | 298.5 |
| | | len = 5349/STRA = for | | | | | | | |
| 12 | 101890_f_at | Cluster Incl U53208 Zuotin related factor 1/cds = (68,1612)/gb = U53208/gi = 1256831/ug = Mm 88678/len = 1995/STRA = for | 381.4 | P | 385.8 | P | 167.8 | P | 331.8 |
| 12 | 101894_s_at | Cluster Incl X71978 Fused toes/cds = (213,1091)/gb = X71978/gi = 311631/ug = Mm.88777/len = 1793/STRA = for | 283.8 | P | 275.3 | P | 208.4 | P | 180.7 |
| 12 | 102645_at | Cluster Incl AI155285.ud84b11 r1 Mus musculus cDNA, 5 end/clone = IMAGE-1477533/clone_end = 5/gb = AI155285/ | 169.3 | P | 145.4 | P | 65 | P | 70.1 |
| | | gi = 3683754/ug = Mm 13146/len = 325/STRA = for | | | | | | | |
| 12 | 102647 _g_at | Cluster Incl AW122284 UI-M-BH2 2-aos-b-02-0-UI s1 Mus musculus cDNA, 3 end/clone = UI-M-BH2 2-aos-b-02-0-UI/ | 730.1 | P | 631.6 | P | 401.9 | P | 507.5 |
| | | clone_end = 3/gb = AW122284/gi = 8097765/ug = Mm 13148/len = 336/STRA = for | | | | | | | |
| 12 | 103300_at | Cluster Incl AW124239 UI-M-BH2 1-aph-e-12-0-UI.s1 Mus musculus cDNA, 3 end/clone = UI-M-BH2.1-aph-e-12-0-UI/ | 571.8 | P | 272.6 | P | 315.1 | P | 458.5 |
| | | clone_end = 3/gb = AW124239/gi = 6099769/ug = Mm 21081/len = 279/STRA = for | | | | | | | |
| 12 | 103309_at | Cluster Incl AI853977 UI-M-BH0-aiv-f-01-0-UI s1 Mus musculus cDNA, 3 end/clone = UI-M-BH0-aiv-f-01-0-UI/ | 322.6 | P | 358.2 | P | 220.5 | P | 277.1 |
| | | clone_end = 3/gb = AI853977/gi = 5497883/ug = Mm 21158/len = 265/STRA = for | | | | | | | |
| 12 | 103459_at | Cluster Incl AW124544 UI-M-BH2 1-apo-h-11-0-UI s1 Mus musculus cDNA, 3 end/clone = UI-M-BH2.1-apo-h-11-0-UI/ | 657.3 | P | 544.3 | P | 387 | P | 628.5 |
| | | clone end = 3/gb = AW124544/gi = 6100074/ug = Mm.21688/len = 256/STRA = for | | | | | | | |
| 12 | 103500_at | Cluster Incl AI553463: vw56g01 x1 Mus musculus cDNA, 3 end/clone = IMAGE-1247856/clone_end = 3/gb = AI553463/ | 334.7 | P | 273.5 | P | 127.3 | P | 194.7 |
| | | gi = 4485826/ug = Mm 22344/len = 457/STRA = rev | | | | | | | |
| 12 | 103525_at | Cluster Incl AA981725: ua28h11 r1 Mus musculus cDNA, 5 end/clone = IMAGE-1348101/clone_end = 5/gb = AA981725/ | 336.9 | P | 288.7 | P | 262.5 | P | 340.3 |
| | | gi = 3160494/ug = Mm 2290/len = 433/STRA = for | | | | | | | |
| 12 | 104163_at | Cluster Incl AA711002: vt52g08.r1 Mus musculus cDNA, 5 end/clone = IMAGE-1166750/clone_end = 5/gb = AA711002/ | 476 | P | 483.5 | P | 346.8 | P | 358.7 |
| | | gi = 2720920/ug = Mm 27130/len = 377/STRA = for | | | | | | | |
| 12 | 104179_at | Cluster Incl AI788669 uk47h11 x1 Mus musculus cDNA, 3 end/clone = IMAGE-1972197/clone_end = 3/gb = AI788669/ | 444.2 | P | 322.9 | P | 160.9 | P | 204.6 |
| | | gi = 5336385/ug = Mm 27308/len = 503/STRA = for | | | | | | | |
| 12 | 104212_at | Cluster Incl AA607767 vo08g02 r1 Mus musculus cDNA, 5 end/clone = IMAGE-1040882/clone_end = 5/gb = AA607767/ | 318.2 | P | 320.3 | P | 217.1 | P | 282.3 |
| | | gi = 2455202/ug = Mm 27763/len = 509/STRA = for | | | | | | | |
| 12 | 104399_at | Cluster Incl AI850965 UI-M-BH0-akd-f-12-0-UI s1 Mus musculus cDNA, 3 end/clone = UI-M-BH0-akd-f-12-0-UI/ | 330.7 | P | 296.1 | P | 215.7 | P | 293.9 |
| | | clone_end = 3/gb = AI850965/gi = 5494871/ug = Mm 2866/len = 323/STRA = for | | | | | | | |
| 12 | 104404_at | Cluster Incl AA693221 vr58c04 s1 Mus musculus cDNA, 5 end/clone = IMAGE-1124838/clone_end = 5/gb = AA693221/ | 354 | P | 256.6 | P | 149 | P | 193.2 |
| | | gi = 2694159/ug = Mm 28701/len = 453/STRA = for | | | | | | | |
| 12 | 92243_at | Cluster Incl AA117417: mp90d10.r1 Mus musculus cDNA, 5 end/clone = IMAGE-576499/clone_end = 5/gb = AA117417/ | 547 | P | 481 | P | 315 | P | 392.1 |
| | | gi = 1672496/ug = Mm.34910/len = 494/STRA = for | | | | | | | |
| 12 | 92282_at | Cluster lncl AI646301: vs77f11.x1 Mus musculus cDNA, 3 end/clone = IMAGE-1152333/clone_end = 3/gb = AI646301/ | 235.5 | P | 299.2 | P | 174.1 | P | 192.1 |
| | | gi = 4724776/ug = Mm 3702/len = 305/STRA = for | | | | | | | |
| 12 | 92360_at | Cluster Incl U43900.Signal transducing adaptor molecule (SH3 domain and ITAM motif) 1/cds = (42,1688)/gb = U43900/ | 508.1 | P | 463.3 | P | 341.8 | P | 469.8 |
| | | gi = 1556460/ug = Mm 3965/len = 2747/STRA = for | | | | | | | |
| 12 | 92480_f_at | Cluster Incl AB024004.Mus musculus mRNA for KRAB-containing zinc-finger protein KRAZ1, complete cds/ | 213.3 | P | 142.1 | P | 61.7 | P | 140.6 |
| | | cds = (116,2074)/gb = AB024004/gi = 4514558/ug = Mm.42140/len = 2191/STRA = for | | | | | | | |
| 12 | 92487_at | Cluster Incl AB023419 Mus musculus mRNA for mSox7, complete cds/cds = (50,1192)/gb = AB023419/gi = 5103026/ | 698.1 | P | 548.8 | P | 530.2 | P | 603.9 |
| | | ug = Mm 42162/len = 3266/STRA = for | | | | | | | |
| 12 | 93426_at | Cluster Incl AA199023 mU13a03 r1 Mus musculus cDNA, 5 end/clone = IMAGE-639244/clone_end = 5/gb = AA199023/ | 611.9 | P | 440.2 | P | 193.4 | P | 298.5 |
| | | gi = 1794518/ug = Mm 6488/len = 203/STRA = for | | | | | | | |
| 12 | 93455_s_at | Cluster Incl X56848.M musculus mRNA for bone morphogenetic protein 4 (BMP-4)/cds = (356,1582)/gb = X56848/gi = 50180/ | 578.9 | P | 582 | P | 348.7 | P | 470.5 |
| | | ug = Mm.6813/len = 1785/STRA = for | | | | | | | |
| 12 | 93471_at | Cluster Incl AI594427-vo35a08 y1 Mus musculus cDNA, 5 end/clone = IMAGE-1051882/clone_end = 5/gb = AI594427/ | 307.6 | P | 264.3 | P | 173 | P | 245.8 |
| | | gI = 4603475/ug = Mm 7033/len = 447/STRA = for | | | | | | | |
| 12 | 93701_at | Cluster Incl AA982124.vx56f01.r1 Mus musculus cDNA, 5 end/clone = IMAGE-1279225/clone_end = 5/gb = AA982124/ | 320 | P | 193.3 | P | 204.5 | P | 196.6 |
| | | gi = 3160243/ug = Mm.86261/len = 474/STRA = for | | | | | | | |
| 12 | 93868_at | Cluster Incl AL021127 Mus musculus cosmid CMX137 containing part of Nsdhl/cds = (0,578)/gb = AL021127/gi = 2769543/ | 167.3 | P | 178.7 | P | 163.6 | P | 203.9 |
| | | ug = Mm 87851/len = 579/STRA = for | | | | | | | |
| 12 | 94110_f_at | Cluster Incl D10627.Mus musculus mRNA, for zinc finger protein, partial cds/cds = (0,1040)/gb = D10627/gi = 220636/ | 514.6 | P | 260.4 | P | 127.5 | P | 215.3 |
| | | ug = Mm 10509/len = 1041/STRA = for | | | | | | | |
| 12 | 95282_at | Cluster Incl J04633 Mouse heat shock protein 86 mRNA, complete cds, and 28S ribosomal RNA, partial sequence/ | 2270.9 | P | 1808.6 | P | 990.1 | P | 1478.5 |
| | | cds = (260,2461)/gb = J04633/gi = 194030/ug = Mm 1843/len = 2975/STRA = for | | | | | | | |
| 12 | 100760_at | Cluster lncl X97986 M musculus mRNA for desmocollin type 1/cds = (151,2649)/gb = X97986/gi = 1707586/ug = Mm 909/ | 728.1 | P | 537.3 | P | 457.6 | P | 618.7 |
| | | len = 2987/STRA = for | | | | | | | |
| 12 | 101634_at | Cluster Incl M33212 Mouse nucleolar protein N038 mRNA, complete cds/cds = (78,956)/gb = M33212/gi = 200010/ | 3209.7 | P | 2823.1 | P | 2447.2 | P | 2803.4 |
| | | ug = Mm 6343/len = 1260/STRA = for | | | | | | | |
| 12 | 92592_at | Cluster Incl M25558 Glycerolphosphate dehydrogenase 1, cytoplasmic adult/cds = (0,1049)/gb = M25558/gi = 193601/ | 840.4 | P | 981.4 | P | 530.3 | P | 702.1 |
| | | ug = Mm 10669/len = 1050/STRA = for | | | | | | | |
| 12 | 92871_at | Cluster Incl AW121840: UI-M-BH2 3-aoi-a-03-0-UI s1 Mus musculus cDNA, 3 end/clone = UI-M-BH2 3-aoi-a-03-0-UI/ | 275.8 | P | 226.9 | P | 239.2 | P | 237.1 |
| | | clone_end = 3/gb = AW121840/gi = 6097214/ug = Mm 14352/len = 466/STRA = for | | | | | | | |
| 12 | 95416_at | Cluster Incl AI642184.uc46h07.x1 Mus musculus cDNA, 3 end/clone = IMAGE-1401085/clone_end = 3/gb = AI642184/ | 240.9 | P | 206.2 | P | 179 | P | 258.5 |
| | | gi = 4720659/ug = Mm.24280/len = 642/STRA = for | | | | | | | |
| 12 | 97273_at | Cluster Incl AI845953: UI-M-AK1-aex-h-04-0-UI s1 Mus musculus cDNA, 3 end/clone = UI-M-AK1-aex-h-04-0-UI/ | 560 | P | 343.4 | P | 330.8 | P | 513.4 |
| | | clone_end = 3/gb = AI845953/gi = 5489859/ug = Mm 27932/len = 357/STRA = for | | | | | | | |
| 12 | 96861_at | Cluster Incl AI852376.UI-M-BH0-ajo-e-04-0-UI.s1 Mus musculus cDNA, 3 end/clone = UI-M-BH0-ajo-e-04-0-UI/ | 204.3 | P | 148.9 | P | 153.1 | A | 276.9 |
| | | clone_end = 3/gb = AI852376/gi = 5496282/ug = Mm 30052/len = 348/STRA = for | | | | | | | |
| 12 | 97506_at | Cluster Incl AI043016 uc87a04.x1 Mus musculus cDNA, 3 end/clone = IMAGE-1432590/clone_end = 3/gb = AI043016/ | 288.4 | P | 205.1 | P | 136.1 | P | 160.2 |
| | | gi = 3286276/ug = Mm 31512/len = 532/STRA = for | | | | | | | |
| 12 | 98135_r_at | Cluster Incl AI851008.UI-M-BH0-ajv-c-01-0-UI.s1 Mus musculus cDNA, 3 end/clone = UI-M-BH0-ajv-c-01-0-UI/ | 59.9 | P | 48.8 | P | 38.6 | P | 45.6 |
| | | clone_end = 3/gb = AI851008/gi = 5494914/ug = Mm.35492/len = 438/STRA = for | | | | | | | |
| 12 | 99662_at | Cluster Incl AI194767 ui56d10.x1 Mus musculus cDNA, 3 end/clone = IMAGE-1886419/clone_end = 3/gb = AI194767/ | 200.8 | P | 170.7 | P | 90.9 | P | 158.7 |
| | | gi = 3747373/ug = Mm 4379/len = 525/STRA = for | | | | | | | |
| 12 | 101020_at | Cluster Incl AI842667 UI-M-AO1-aeo-a-04-0-UI.s1 Mus musculus cDNA, 3 end/clone = UI-M-AO1-aeo-a-04-0-UI/ | 234.6 | P | 101.6 | P | 141 | P | 158 |
| | | clone_end = 3/gb = AI842667/gi = 5476880/ug = Mm.684/len = 284/STRA = for | | | | | | | |
| 12 | 101079_at | Cluster Incl AF093140 Mus musculus tip associating protein (Tap) mRNA, complete cds/cds = (105,1961)/gb = AF093140/ | 169.3 | P | 132.1 | P | 149.1 | P | 163.9 |
| | | gi = 3719434/ug = Mm 7271/len = 2034/STRA = for | | | | | | | |
| 12 | 103063_at | Cluster Incl Z67747 M. musculus mRNA for ZT3 zinc finger factor/cds = (0,1570)/gb = Z67747/gi = 1061193/ug = Mm 16650/ | 156 | P | 97.7 | P | 16.7 | A | 83.8 |
| | | len = 2547/STRA = for | | | | | | | |
| 12 | 103598_at | Cluster Incl U91922: D-E-A-D (aspartate-glutamate-alanine-aspartate) box polypeptide 9/cds = (107,4249)/gb = U91922/ | 278.1 | P | 225.8 | P | 140.7 | P | 237.2 |
| | | gi = 2961455/ug = Mm 20000/len = 4498/STRA = for | | | | | | | |
| 12 | 103665_at | Cluster Incl AW122523 UI-M-BH2.2-aox-b-05-0-UI s1 Mus musculus cDNA, 3 end/clone = UI-M-BH2 2-aox-b-05-0-UI/ | 588.8 | P | 336 | P | 211.2 | P | 264.1 |
| | | clone_end = 3/gb = AW122523/gi = 6098047/ug = Mm 20697/len = 423/STRA = for | | | | | | | |
| 12 | 104298_at | Cluster Incl AI842544 UI-M-AQ1-aea-e-05-0-UI.s1 Mus musculus cDNA, 3 end/clone = UI-M-AQ1-aea-e-05-0-UI/ | 168.3 | P | 141.3 | P | 58.6 | A | 129.3 |
| | | clone end = 3/gb = AI842544/gi = 5476757/ug = Mm 22337/len = 457/STRA = for | | | | | | | |
| 12 | 104536_at | Cluster Incl U60530 MAD homolog 2, (Drosophila)/cds = (59,1462)/gb = U60530/gi = 1407781/ug = Mm 2334/len = 1639/ | 414.7 | P | 357.2 | P | 283 | P | 255.5 |
| | | STRA = for | | | | | | | |
| 12 | 95521_s_at | Cluster Incl AB024005 Mus musculus mRNA for KRAB-containing zinc-finger protein KRAZ2, complete cds/ | 162.4 | P | 137.9 | P | 93.7 | A | 138.2 |
| | | cds = (307,2127)/gb = AB024005/gi = 4514560/ug = Mm 27575/len = 2247/STRA = for | | | | | | | |
| 12 | 100459_at | Cluster Incl U66887: RAD50 homolog (S cerevisiae)/cds = (242,4180)/gb = U66887/gi = 1575574/ug = Mm 4888/len = 5088/ | 228.3 | P | 196.6 | P | 82.7 | A | 132.5 |
| | | STRA = for | | | | | | | |
| 12 | 102872_f_at | Cluster Incl X74855 Zinc finger protein 51/cds = (350,2482)/gb = X74855/gi = 488832/ug = Mm 14812/len = 2640/STRA = for | 166.1 | P | 94.9 | P | 44 | P | 53.5 |
| 12 | 102887_at | Cluster Incl U94331 Mus musculus osteoprotegerin (OPG) mRNA, complete cds/cds = (90,1295)/gb = U94331/gi = 2072182/ | 403.6 | P | 184.3 | P | 347.8 | P | 198.3 |
| | | ug = Mm 15383/len = 1325/STRA = for | | | | | | | |
| 12 | 103745_at | Cluster Incl AW227027.um65d05 y1 Mus musculus cDNA, 5 end/clone = IMAGE-2286249/clone_end = 5/gb = AW227027/ | 170.1 | P | 136.2 | P | 87.4 | P | 119.7 |
| | | gi = 6556323/ug = Mm.24475/len = 457/STRA = for | | | | | | | |
| 12 | 104419_at | Cluster Incl AI132380 ue24a03.x1 Mus musculus cDNA, 3 end/clone = IMAGE-1481260/clone_end = 3/gb = AI132380/ | 159.2 | P | 100.7 | P | 18.5 | P | 116.1 |
| | | gi = 3602396/ug = Mm.28928/len = 441/STRA = for | | | | | | | |
| 12 | 104501_at | Cluster Incl AI849048 UI-M-AH1-agw-b-07-0-UI.s1 Mus musculus cDNA, 3 end/clone = UI-M-AH1-agw-b-07-0-UI/ | 110.3 | P | 85.4 | P | 84.8 | P | 104.8 |
| | | clone_end = 3/gb = AI849048/gi = 5492954/ug = Mm 3055/len = 421/STRA = rev | | | | | | | |
| 12 | 92408_at | Cluster Incl AI891514: ul59e08.x1 Mus musculus cDNA, 3 end/clone = IMAGE-2123366/clone_end = 3/gb = AI891514/ | 205.3 | P | 111.7 | P | 57.5 | P | 138.2 |
| | | gi = 5597416/ug = Mm.41046/len = 479/STRA = for | | | | | | | |
| 12 | 92649_at | Cluster Incl Y18505: Mus musculus mRNA for XAP-5 protein/cds = (147,1166)/gb = Y18505/gi = 5327064/ug = Mm.4370/ | 220.6 | P | 263.5 | P | 168.6 | P | 210.2 |
| | | len = 1348/STRA = for | | | | | | | |
| 12 | 95289_f_at | Cluster Incl AA189811: mt97g08 r1 Mus musculus cDNA, 5 end/clone = IMAGE-637886/clone_end = 5/gb = AA189811/ | 63.6 | P | 49 | P | 26 | P | 37.6 |
| | | gi = 1776416/ug = Mm.1882/len = 365/STRA = for | | | | | | | |
| 12 | 98761_i_at | Cluster Incl L20450 Mus musculus DNA-binding protein mRNA, complete cds/cds = (93,1733)/gb = L20450/gi = 437443/ | 350.9 | P | 104.5 | P | 19.3 | P | 109.5 |
| | | ug = Mm 4596/len = 2768/STRA = for | | | | | | | |
| 12 | 101294_g_at | Cluster Incl Z84471 M musculus G6pd-2 gene/cds = (0,1541)/gb = Z84471/gi = 1806125/ug = Mm 56918/len = 1542/ | 398 | P | 265.5 | P | 216.3 | A | 308.2 |
| | | STRA = for | | | | | | | |
| 12 | 93568_i_at | Cluster Incl AI853444 UI-M-BH0-ajy-d-09-0-UI s1 Mus musculus cDNA, 3 end/clone = UI-M-BH0-ajy-d-09-0-UI/ | 147.4 | P | 67 | P | 8.7 | A | 65.2 |
| | | clone_end = 3/gb = AI853444/gi = 5497350/ug = Mm 204/len = 363/STRA = for | | | | | | | |
| 12 | 94009_at | Cluster Incl AI848888 UI-M-AJ1-ahb-g-12-0-UI s1 Mus musculus cDNA, 3 end/clone = UI-M-AJ1-ahb-g-12-0-UI/ | 119.4 | P | −8.8 | P | −12.2 | P | 7.4 |
| | | clone_end = 3/gb = AI848888/gi = 5492794/ug = Mm.21811/len = 365/STRA = for | | | | | | | |
| 12 | 94917_at | Cluster Incl AI844932 UI-M-BG0-ahv-f-12-0-UI s1 Mus musculus cDNA, 3 end/clone = UI-M-BG0-ahv-f-12-0-UI/ | 79.8 | P | 106.7 | P | 87.3 | P | 99.6 |
| | | clone_end = 3/gb = AI844932/gi = 5488838/ug = Mm 24156/len = 407/STRA = for | | | | | | | |
| 12 | 95483_at | Cluster Incl AW123318 UI-M-BH2.1-apf-b-03-0-UI.s1 Mus musculus cDNA, 3 end/clone = UI-M-BH2 1-apf-b-03-0-UI/ | 291.2 | P | 477.8 | P | 300.1 | P | 347 |
| | | clone_end = 3/gb = AW123318/gi = 6098848/ug = Mm 24874/len = 371/STRA = for | | | | | | | |
| 12 | 96052_at | Cluster Incl AI852457 UI-M-BH0-aip-e-04-0-UI s1 Mus musculus cDNA, 3 end/clone = UI-M-BH0-aip-e-04-0-UI/ | 371.1 | P | 274.3 | P | 168.1 | P | 269.4 |
| | | clone_end = 3/gb = AI852457/gi = 5496363/ug = Mm 26166/len = 426/STRA = for | | | | | | | |
| 12 | 97243_at | Cluster Incl U74079 Mus musculus protein co-factor mRNA, complete cds/cds = (137,1204)/gb = U74079/gi = 1644403/ | 968.7 | P | 1055.4 | P | 874.3 | P | 963.6 |
| | | ug = Mm 27842/len = 1901/STRA = for | | | | | | | |
| 12 | 98959_at | Cluster Incl AW120623 UI-M-BH2.3-anx-h-09-0-UI s1 Mus musculus cDNA, 3 end/clone = UI-M-BH2 3-anx-h-09-0-UI/ | 328.5 | P | 313.6 | A | 218.9 | P | 275.9 |
| | | clone_end = 3/gb = AW120623/gi = 6095956/ug = Mm 28773/len = 308/STRA = for | | | | | | | |
| 12 | 94526_at | Cluster Incl AI848453 UI-M-AH1-ags-f-11-0-UI s1 Mus musculus cDNA, 3 end/clone = UI-M-AH1-ags-f-11-0-UI/ | 423 | P | 288.5 | P | 211.9 | P | 255.3 |
| | | clone_end = 3/gb = AI848453/gi = 5492359/ug = Mm.29032/len = 495/STRA = for | | | | | | | |
| 12 | 98539_at | Cluster Incl AF071312 Mus musculus COP9 complex subunit 2 (COPS2) mRNA, complete cds/cds = (12,1334)/ | 101.3 | P | 96.3 | P | 40.5 | P | 56.4 |
| | | gb = AF071312/gi = 3309165/ug = Mm 3596/len = 1929/STRA = for | | | | | | | |
| 12 | 103321_at | Cluster Incl AI536457 vu57h06 x1 Mus musculus cDNA, 3 end/clone = IMAGE-1195547/clone_end = 3/gb = AI536457/ | 128.6 | P | 136.6 | P | 50.3 | P | 129.6 |
| | | gi = 4450592/ug = Mm 17873/len = 435/STRA = for | | | | | | | |
| 12 | 104285_at | Cluster Incl M62766 3-hydroxy-3-methylglutaryl-coenzyme A reductase/cds = (0,674)/gb = M62766/gi = 193881/ | 227.3 | P | 237.5 | P | 84.2 | P | 259.1 |
| | | ug = Mm 2226/len = 1045/STRA = for | | | | | | | |
| 12 | 98966_at | Cluster Incl L42996 Mus musculus nuclear-encoded mitochondrial acyltransferase mRNA, complete cds/cds = (15,1463)/ | 136.3 | P | 49.9 | P | 5.8 | P | 84.3 |
| | | gb = L42996/gi = 1100214/ug = Mm 3636/len = 2664/STRA = for | | | | | | | |
| 12 | 100512_at | Cluster Incl AI838853: UI-M-AN0-ack-b-11-0-UI s1 Mus musculus cDNA, 3 end/clone = UI-M-AN0-ack-b-11-0-UI/ | 294.4 | P | 274.9 | P | 109.8 | P | 262.9 |
| | | clone_end = 3/gb = AI838853/gi = 5473066/ug = Mm 52067/len = 397/STRA = for | | | | | | | |
| 12 | 102197_at | Cluster Incl AJ222586 Mus musculus mRNA for NEFA protein, complete cds/cds = (287,1549)/gb = AJ222586/gi = 2612796/ | 95 | P | 60.6 | P | 66.2 | P | 74 |
| | | ug = Mm.9901/len = 1655/STRA = for | | | | | | | |
| 12 | 103260_at | Cluster Incl AI838553: UI-M-AL0-abp-d-10-0-UI s1 Mus musculus cDNA, 3 end/clone = UI-M-AL0-abp-d-10-0-UI/ | 104.9 | P | 107.8 | P | 78.1 | P | 83.7 |
| | | clone_end = 3/gb = AI838553/gi = 5472731/ug = Mm 208/len = 491/STRA = rev | | | | | | | |
| 12 | 95318_at | Cluster Incl AF045565 Zinc finger protein 105/cds = (231,1805)/gb = AF045565/gi = 3483132/ug = Mm 20430/len = 2002/ | 281 | P | 208.1 | P | 160.5 | P | 165.9 |
| | | STRA = for | | | | | | | |
| 12 | 96560_at | Cluster Incl AA648027 vn68h05 r1 Mus musculus cDNA, 5 end/clone = IMAGE-1026393/clone_end = 5/gb = AA648027/ | 696.4 | P | 463.9 | P | 421.8 | P | 424.9 |
| | | gi = 2574456/ug = Mm 28931/len = 419/STRA = for | | | | | | | |
| 12 | 99828_at | Cluster Incl AA673252 vp49C07 r1 Mus musculus cDNA, 3 end/clone = IMAGE-1080012/clone_end = 3/gb = AA673252/ | 125.7 | M | 190.2 | P | 70.2 | A | 152.6 |
| | | gi = 2646534/ug = Mm 6148/len = 370/STRA = rev | | | | | | | |
| 12 | 93502_r_at | Cluster Incl AF058955 Mus musculus ATP-specific succinyl-CoA synthetase beta subunit (Scs) mRNA, partial cds/ | 211.7 | P | 131.5 | M | 112.4 | P | 157.2 |
| | | cds = (0,1280)/gb = AF058955/gi = 3766200/ug = Mm 19154/len = 1991/STRA = for | | | | | | | |
| 12 | 95660_at | Cluster Incl AI851815: UI-M-BH0-ais-c-07-0-UI s1 Mus musculus cDNA, 3 end/clone = UI-M-BH0-ais-c-07-0-UI/ | 195.2 | P | 125.9 | P | 93 | P | 267 |
| | | clone_end = 3/gb = AI851815/gi = 5495721/ug = Mm 29553/len = 341/STRA = for | | | | | | | |
| 12 | 98073_at | Cluster Incl U66249 Mus musculus Cut alternatively spliced product (CASP) mRNA, partial cds/cds = (0,957)/gb = U66249/ | 447.6 | P | 278.9 | P | 314.1 | P | 215.4 |
| | | gi = 1518859/ug = Mm.34465/len = 1757/STRA = for | | | | | | | |
| 12 | 101524_at | Cluster Incl AI851595 UI-M-BH0-aki-h-08-0-UI.s1 Mus musculus cDNA, 3 end/clone = UI-M-BH0-aki-h-08-0-UI/ | 81.8 | P | 89.8 | M | 51.3 | P | 62.7 |
| | | clone_end = 3/gb = AI851595/gi = 5495501/ug = Mm 86187/len = 521/STRA = for | | | | | | | |
| 12 | 102982_at | Cluster Incl AI854581 UI-M-BH0-ake-a-06-0-UI.s1 Mus musculus cDNA, 3 end/clone = UI-M-BH0-ake-a-06-0-UI/ | 254.4 | P | 206.7 | P | 49.3 | A | 172.1 |
| | | clone_end = 3/gb = AI854581/gi = 5498487/ug = Mm 1511/len = 562/STRA = for | | | | | | | |
| 12 | 96148_at | Cluster Incl AI844238 UI-M-AL1-ahk-f-10-0-UI s1 Mus musculus cDNA, 3 end/clone = UI-M-AL1-ahk-f-10-0-UI/ | 146.6 | P | 101.7 | P | 34.8 | A | 127.5 |
| | | clone_end = 3/gb = AI844238/gi = 5488144/ug = Mm 28232/len = 395/STRA = rev | | | | | | | |
| 12 | 96165_at | Cluster Incl AW125109 UI-M-BH2.1-apx-h-05-0-UI s1 Mus musculus cDNA, 3 end/clone = UI-M-BH2 1-apx-h-05-0-UI/ | 337.2 | P | 299.1 | P | 181.3 | A | 207.6 |
| | | clone_end = 3/gb = AW125109/gi = 6100639/ug = Mm.28370/len = 376/STRA = rev | | | | | | | |
| 12 | 99923_at | Cluster Incl AA615429: vo78b10.r1 Mus musculus cDNA, 5 end/clone = IMAGE-1065211/clone_end = 5/gb = AA615429/ | 69.3 | P | 57.1 | A | 30.6 | A | 35.3 |
| | | gi = 2502657/ug = Mm.43157/len = 375/STRA = for | | | | | | | |
| 12 | 104418_at | Cluster Incl AA796868: vp19h01.r1 Mus musculus cDNA, 5 end/clone = IMAGE-1069105/clone_end = 5/gb = AA796868/ | 105.9 | P | 77.7 | A | 29.4 | M | 46.1 |
| | | gi = 2859823/ug = Mm 28920/len = 343/STRA = for | | | | | | | |
| 12 | 93175_at | Cluster Incl AI049206 ub39g05 r1 Mus musculus cDNA, 5 end/clone = IMAGE-1380152/clone_end = 5/gb = AI049206/ | 262.1 | P | 184.4 | A | 209.8 | P | 266.9 |
| | | gi = 3297493/ug = Mm 5357/len = 501/STRA = for | | | | | | | |
| 12 | 92806_at | Cluster Incl AI843868 UI-M-AK1-aeu-d-04-0-UI.s1 Mus musculus cDNA, 3 end/clone = UI-M-AK1-aeu-d-04-0-UI/ | 465.5 | P | 434.6 | P | 232.5 | A | 381.1 |
| | | clone_end = 3/gb = AI843868/gi = 5478081/ug = Mm 12746/len = 408/STRA = for | | | | | | | |
| 12 | 93569_f_at | Cluster Incl AI853444 UI-M-BH0-ajy-d-09-0-UI s1 Mus musculus cDNA, 3 end/clone = UI-M-BH0-ajy-d-09-0-UI/ | 62.3 | P | 43.3 | P | 2.8 | A | 37 |
| | | clone_end = 3/gb = AI853444/gi = 5497350/ug = Mm.204/len = 363/STRA = for | | | | | | | |
| 12 | 94239_at | Cluster Incl Y08701 M. musculus mRNA for Pinin protein/cds = (18,2195)/gb = Y08701/gi = 2570048/ug = Mm 22347/ | 192.3 | P | 94.1 | P | 52.6 | P | 44.8 |
| | | len = 2813/STRA = for | | | | | | | |
| 12 | 98944_at | Cluster Incl AI848343: UI-M-AH1-ago-c-12-0-U1.s1 Mus musculus cDNA, 3 end/clone = UI-M-AH1-ago-c-12-0-UI/ | 322.6 | P | 198 | P | 119.4 | A | 384.6 |
| | | clone_end = 3/gb = AI848343/gi = 5492249/ug = Mm.28704/len = 491/STRA = for | | | | | | | |
| 12 | 103346_at | Cluster Incl AF033564: Mus musculus cdc2/CDC28-like kinase 2 (Clk2) mRNA, complete cds/cds = (24,1523)/ | 350.9 | P | 301 | P | 163.7 | M | 232.8 |
| | | gb = AF033564/gi = 2645851/ug = Mm 18268/len = 1538/STRA = rev | | | | | | | |
| 12 | 104088_at | Cluster Incl AW060311 UI-M-BH1-amv-d-09-0-UI s1 Mus musculus cDNA, 3 end/clone = UI-M-BH1-amv-d-09-0-UI/ | 259.5 | P | 255.3 | P | 271.5 | P | 230.7 |
| | | clone_end = 3/gb = AW060311/gi = 6008027/ug = Mm.21795/len = 443/STRA = for | | | | | | | |
| 12 | 104112_at | Cluster Incl AI854033 UI-M-BH0-ajm-c-10-0-UI.s1 Mus musculus cDNA, 3 end/clone = UI-M-BH0-ajm-c-10-0-UI/ | 215.7 | P | 107.7 | A | 95.4 | P | 206.7 |
| | | clone_end = 3/gb = AI854033/gi = 5497939/ug = Mm.21936/len = 468/STRA = for | | | | | | | |
| 12 | 100988_at | Cluster Incl AA796690 vp15a12 rt Mus musculus cDNA, 5 end/clone = IMAGE-1068670/clone_end = 5/gb = AA796690/ | 183.4 | P | 129.8 | M | 72.4 | A | 173.2 |
| | | gi = 2859645/ug = Mm 6807/len = 403/STRA = for | | | | | | | |
| 12 | 102678_at | Cluster Incl L27990 Mus musculus Ro protein mRNA, complete cds/cds = (140,1552)/gb = L27990/gi = 825145 ug = Mm 1384/ | 248.7 | P | 296.5 | A | 210.2 | P | 228.3 |
| | | len = 1840/STRA = for | | | | | | | |
| 12 | 104216_at | Cluster Incl AF068921 Mus musculus Ras-binding protein SUR-8 mRNA, complete cds/cds = (0,1748)/gb = AF068921/ | 179.1 | A | 128 | P | 73.5 | A | 118.2 |
| | | gi = 3252980/ug = Mm 27860/len = 1749/STRA = for | | | | | | | |
| 12 | 93112_at | Cluster Incl D86725 Mouse mRNA for mMCM2, complete cds/cds = (50,2764)/gb = D86725/gi = 2381484/ug = Mm 16711/ | 294.6 | P | 224.9 | P | 142.9 | A | 236.9 |
| | | len = 3290/STRA = for | | | | | | | |
| 12 | 94829_at | Cluster Incl AW121838 UI-M-BH2 3-anx-h-10-0-UI s2 Mus musculus cDNA, 3 end/clone = UI-M-BH2 3-anx-h-10-0-UI/ | 290.2 | P | 265.7 | P | 249.7 | A | 256.7 |
| | | clone_end = 3/gb = AW121838/gi = 6097212/ug = Mm 22746/len = 319/STRA = for | | | | | | | |
| 12 | 97470_at | Cluster Incl D86424 Mus musculus mRNA for high-sulfur keratin protein, partial cds/cds = (0,291)/gb = D86424/gi = 1944400/ | 783.4 | P | 103.1 | A | −135.6 | A | 856.6 |
| | | ug = Mm 30539/len = 409/STRA = for | | | | | | | |
| 12 | 98973_at | Cluster Incl AA982595: uh10e10 r1 Mus musculus cDNA, 5 end/clone = IMAGE-1617546/clone_end = 5/gb = AA982595/ | 262.4 | P | 179 | A | 128.7 | A | 178.2 |
| | | gi = 3161254/ug = Mm 36583/len = 407/STRA = for | | | | | | | |
| 12 | 100947_at | Cluster Incl AI847906: UI-M-AI1-afl-a-11-0-UI s1 Mus musculus cDNA, 3 end/clone = UI-M-AI1-afl-a-11-0-UI/clone_end = 3/ | 170.9 | P | 65 | A | 48.3 | A | 146.9 |
| | | gb = AI847906/gi = 5491812/ug = Mm 640/len = 510/STRA = for | | | | | | | |
| 12 | 103957_at | Cluster Incl X57349 M musculus mRNA for transfemn receptor/cds = (0,2291)/gb = X57349/gi = 54914/ug = Mm 25560/ | 75.9 | A | 90 | P | 34.4 | A | 60.3 |
| | | len = 2292/STRA = for | | | | | | | |
| 12 | 104427_at | Cluster Incl AA930526: vo45f08 r1 Mus musculus cDNA, 5 end/clone = IMAGE-1052871/clone_end = 5/gb = AA930526/ | 341 | P | 346.7 | A | 119.2 | P | 320.7 |
| | | gi = 3080016/ug = Mm 29172/len = 410/STRA = for | | | | | | | |
| 12 | 104468_at | Cluster lncl AW045530: UI-M-BH1-aks-c-05-0-UI s1 Mus musculus cDNA, 3 end/clone = UI-M-BH1-aks-c-05-0-UI/ | 150.6 | P | 89.2 | P | 54 | M | 126.2 |
| | | clone_end = 3/gb = AW045530/gi = 5906059/ug = Mm 29755/len = 391/STRA = for | | | | | | | |
| 12 | 104694_at | Cluster Incl AI152353: ud79d01.r1 Mus musculus cDNA, 5 end/clone = IMAGE-1477057/clone_end = 5/gb = AI152353/ | 234.7 | P | 172.3 | A | 94.2 | A | 137.6 |
| | | gi = 3680822/ug = Mm 3347/len = 386/STRA = for | | | | | | | |
| 12 | 102102 _at | Cluster Incl C80410 C80410 Mus musculus cDNA, 3 end/clone = J0081D08/clone_end = 3/gb = C80410/gi = 2520740/ | 109.5 | P | 112.8 | P | 70.9 | P | 73.3 |
| | | ug = Mm 42447/len = 560/STRA = rev | | | | | | | |
| 12 | 95655_at | Cluster Incl AA717740 vt98b12 r1 Mus musculus cDNA, 5 end/clone = IMAGE-1179187/clone_end = 5/gb = AA717740/ | 60.8 | P | 39.2 | A | 7.9 | A | 58.7 |
| | | gi = 2730014/ug = Mm 29540/len = 283/STRA = for | | | | | | | |
| 12 | 99570_s_at | Cluster Incl AF029982 Mus musculus sarco(endo)plasmic reticulum calcium ATPase (SERCA2) gene, promoter region, | 4.5 | A | −98.4 | P | −39.6 | P | −51 |
| | | exons 1-3, and partial cds/cds = (523,741)/gb = AF029982/gi = 4103931/ug = Mm 42255/len = 742/STRA = for | | | | | | | |
| 12 | 100565_at | Cluster Incl AW123396 UI-M-BH2.1-apf-h-12-0-UI s1 Mus musculus cDNA, 3 end/clone = UI-M-BH2.1-apf-h-12-0-UI/ | 162.3 | P | 94.6 | P | 52.9 | A | 124.2 |
| | | clone_end = 3/gb = AW123396/gi = 6098926/ug = Mm 5736/len = 420/STRA = for | | | | | | | |
| 12 | 101515_at | Cluster Incl AF006688: Mus musculus peroxisomal acyl-CoA oxidase (muspaox) mRNA, complete cds/cds = (80,2065)/ | 163.2 | M | 154.2 | P | 76.6 | A | 121.6 |
| | | gb = AF006688/gi = 2253379/ug = Mm 8403/len = 3778/STRA = for | | | | | | | |
| 12 | 102004_at | Cluster Incl AI530403 ui92f02.y1 Mus musculus cDNA, 5 end/clone = IMAGE-1889883/clone_end = 5/gb = AI530403/ | 149.4 | P | −81.2 | A | −196.6 | A | −122.2 |
| | | gi = 4444538/ug = Mm 99132/len = 906/STRA = for | | | | | | | |
| 12 | 103033_at | Cluster Incl X06454 Mouse mRNA for sex-limited protein Slp(w7) alpha-gamma chain/cds = (0,1784)/gb = X06454/gi = 54105/ | 309.8 | P | 164.5 | P | 92.4 | A | 201.4 |
| | | ug = Mm 16106/len = 1893/STRA = for | | | | | | | |
| 12 | 103427_at | Cluster Incl AW123223 UI-M-BH2.1-apg-e-07-0-UI s1 Mus musculus cDNA, 3 end/clone = UI-M-BH2 1-apg-e-07-0-UI/ | 238.7 | P | 182.9 | A | 82.3 | A | 162 |
| | | clone_end = 3/gb = AW123223/gi = 6098793/ug = Mm.18968/len = 410/STRA = for | | | | | | | |
| 12 | 103658_r_at | Cluster Incl AI836552 UI-M-AP0-abe-e-08-0-UI.s1 Mus musculus cDNA, 3 end/clone = UI-M-AP0-abe-e-08-0-UI/ | 111.3 | P | 88.4 | P | 37.2 | A | 67.2 |
| | | clone_end = 3/gb = AI836552/gi = 5470765/ug = Mm.20559/len = 417/STRA = for | | | | | | | |
| 12 | 100925_at | Cluster Incl AI131982 uc36a02 r1 Mus musculus cDNA, 5 end/clone = IMAGE-1400042/clone_end = 5/gb = AI131982/ | 68.6 | P | 50.5 | A | 48 | A | 34.9 |
| | | gi = 3601998/ug = Mm.608/len = 398/STRA = for | | | | | | | |
| 12 | 103963_f_at | Cluster Incl AA914345: vy94a08 r1 Mus musculus cDNA, 5 end/clone = IMAGE-1313846/clone_end = 5/gb = AA914345/ | 211.9 | P | 79.6 | A | −112.3 | A | 127.7 |
| | | gi = 3053737/ug = Mm.25624/len = 452/STRA = for | | | | | | | |
| 12 | 92338_f_at | Cluster Incl AI957030 ul74a01 x1 Mus musculus cDNA, 3 end/clone = IMAGE-2136264/clone_end = 3/gb = AI957030/ | 76.1 | P | 30.5 | P | 4.5 | A | −1.2 |
| | | gi = 5749739/ug = Mm 39024/len = 550/STRA = for | | | | | | | |
| 12 | 100389_at | Cluster Incl AF021231 Mus musculus acetylcholinesterase-associated collagen (COIQ) gene/cds = (0,710)/gb = AF021231/ | 488.5 | P | 514 | M | 347.7 | M | 452.1 |
| | | gi = 2460054/ug = Mm 89475/len = 711/STRA = for | | | | | | | |
| 13 | 92578_at | Cluster Incl U10118 Mus musculus endothelial-monocyte activating polypeptide II mRNA, complete cds/cds = (63,995)/ | 1017.8 | P | 924.5 | P | 1058.7 | P | 991.2 |
| | | gb = U10118/gi = 498911/ug = Mm.10507/len = 1068/STRA = for | | | | | | | |
| 13 | 92633_at | Cluster Incl AJ242663 Mus musculus mRNA for cathepsin Z precursor (ctsZ gene)/cds = (104,1024)/gb = AJ242663/ | 236.9 | P | 380.4 | P | 395.3 | P | 347.9 |
| | | gi = 5019547/ug = Mm 115/len = 1291/STRA = for | | | | | | | |
| 13 | 92794_f_at | Cluster Incl M35970 Mouse tumor metastatic process-associated protein (NM23) gene mRNA, 3 end/cds = (0,500)/ | 228.2 | P | 116 | P | 176.3 | P | 177.3 |
| | | gb = M35970/gi = 200066/ug = Mm 1260/len = 664/STRA = for | | | | | | | |
| 13 | 92800_i_at | Cluster Incl AI836694: UI-M-AJ0-aax-a-08-0-UI s1 Mus musculus cDNA, 3 end/clone = UI-M-AJ0-aax-a-08-0-UI/ | 3545.4 | P | 3305.4 | P | 3141.5 | P | 3275.8 |
| | | clone_end = 3/gb = AI836694/gi = 5470907/ug = Mm 12677/len = 344/STRA = for | | | | | | | |
| 13 | 92804_at | Cluster Incl AW122864 UI-M-BH2 1-apa-a-02-0-UI s1 Mus musculus cDNA, 3 end/clone = UI-M-BH2 1-apa-a-02-0-UI/ | 418.5 | P | 417.2 | P | 394.1 | P | 469.2 |
| | | clone_end = 3/gb = AW122864/gi = 6098394/ug = Mm 12706/len = 346/STRA = for | | | | | | | |
| 13 | 92807_at | Cluster Incl X77585: Thioredoxin/cds = (74,391)/gb = X77585/gi = 453971/ug = Mm 1275/len = 538/STRA = for | 3344.9 | P | 3186.4 | P | 3237.4 | P | 3280 |
| 13 | 92834_at | Cluster Incl X51528: Tissue specific transplantation antigen P198-7/cds = (18,629)/gb = X51528/gi = 55049/ug = Mm.13020/ | 1075.5 | P | 883 | P | 989.7 | P | 976.4 |
| | | len = 654/STRA = for | | | | | | | |
| 13 | 92848_at | Cluster Incl X64837: M. musculus Oat mRNA for omithine aminotransferase/cds = (114,1433)/gb = X64837/gi = 53458/ | 931.8 | P | 841.6 | P | 941.3 | P | 832.2 |
| | | ug = Mm.13694/len = 2125/STRA = for | | | | | | | |
| 13 | 92884_at | Cluster Incl AW045468: UI-M-BH1-akr-b-07-0-UI s1 Mus musculus cDNA, 3 end/clone = UI-M-BH1-akr-b-07-0-UI/ | 2634.8 | P | 2653.2 | P | 2828.8 | P | 2526.5 |
| | | clone_end = 3/gb = AW045468/gi = 5905962/ug = Mm.14534/len = 318/STRA = for | | | | | | | |
| 13 | 93008_at | Cluster Incl AW120557 UI-M-BH1-ana-f-03-0-UI s2 Mus musculus cDNA, 3 end/clone = UI-M-BH1-ana-f-03-0-UI/ | 808.4 | P | 782.8 | P | 670.2 | P | 668.3 |
| | | clone_end = 3/gb = AW120557/gi = 6095890/ug = Mm 14585/len = 530/STRA = for | | | | | | | |
| 13 | 93019_at | Cluster Incl Z35401 M musculus C3H gene for histone H2A.X/cds = (0,431)/gb = Z35401/gi = 515878/ug = Mm 14767/ | 1812.3 | P | 1901.4 | P | 1741.1 | P | 1305.3 |
| | | len = 432/STRA = for | | | | | | | |
| 13 | 93254_at | Cluster Incl AI839142 UI-M-AO0-ach-d-11-0-UI s1 Mus musculus cDNA, 3 end/clone = UI-M-AO0-ach-d-11-0-UI/ | 1572.3 | P | 1524.8 | P | 1470.6 | P | 1318.1 |
| | | clone_end = 3/gb = AI839142/gi = 5473355/ug = Mm 1700/len = 523/STRA = for | | | | | | | |
| 13 | 93257_at | Cluster Incl AW048287 UI-M-BH1-aly-f-10-0-UI s1 Mus musculus cDNA, 3 end/clone = UI-M-BH1-aly-f-10-0-UI/ | 762.7 | P | 723.5 | P | 657.5 | P | 619.7 |
| | | clone_end = 3/gb = AW048287/gi = 5908816/ug = Mm 1706/len = 402/STRA = for | | | | | | | |
| 13 | 93281_at | Cluster Incl AF049125: Mus musculus taipoxin-associated calcium binding protein 49 mRNA, complete cds/cds = (193,1158)/ | 399.9 | P | 383.2 | P | 335 | P | 303.4 |
| | | gb = AF049125/gi = 2935465/ug = Mm 1782/len = 1790/STRA = for | | | | | | | |
| 13 | 93298_at | Cluster Incl U34883 3-phosphoadenosine 5-phosphosulfate synthase 1/cds = (33,1907)/gb = U34883/gi = 1109675/ | 695.8 | P | 542.9 | P | 498.4 | P | 507.4 |
| | | ug = Mm 18161/len = 2479/STRA = for | | | | | | | |
| 13 | 93326_at | Cluster Incl D26483: Mouse mRNA for PE31/TALLA, complete cds/cds = (5,739)/gb = D26483/gi = 685220/ug = Mm 18590/ | 468.3 | P | 463.3 | P | 523.8 | P | 458.3 |
| | | len = 1720/STRA = for | | | | | | | |
| 13 | 93531_at | Cluster Incl AI853855: UI-M-BH0-ajr-e-09-0-UI s1 Mus musculus cDNA, 3 end/clone = UI-M-BH0-ajr-e-09-0-UI/ | 604.8 | P | 891.6 | P | 769.9 | P | 734.5 |
| | | clone_end = 3/gb = AI853855/gi = 5497761/ug = Mm 19834/len = 540/STRA = for | | | | | | | |
| 13 | 93536_at | Cluster Incl L22472: Bcl2-associated X protein/cds = (0,578)/gb = L22472/gi = 388191/ug = Mm 19904/len = 579/STRA = for | 331.2 | P | 347 | P | 328.2 | P | 317 |
| 13 | 93548_at | Cluster Incl AW122942: UI-M-BH2.1-aoz-a-11-0-UI s2 Mus musculus cDNA, 3 end/clone = UI-M-BH2 1-aoz-a-11-0-UI/ | 1224.1 | P | 1020.8 | P | 815.7 | P | 905.4 |
| | | clone_end = 3/gb = AW122942/gi = 6098472/ug = Mm 20188/len = 540/STRA = for | | | | | | | |
| 13 | 93551_at | Cluster Incl AW121424: UI-M-BH2.2-aon-b-07-0-UI s1 Mus musculus cDNA, 3 end/clone = UI-M-BH2 2-aon-b-07-0-UI/ | 1748.1 | P | 1709.6 | P | 1489.8 | P | 1681.8 |
| | | clone_end = 3/gb = AW121424/gi = 6096757/ug = Mm 20207/len = 418/STRA = for | | | | | | | |
| 13 | 93559_at | Cluster Incl D90374: Mouse mRNA for APEX nuclease, complete cds/cds = (66,1019)/gb = D90374/gi = 220336/ug = Mm 203/ | 776.7 | P | 811.8 | P | 794.2 | P | 799.3 |
| | | len = 1185/STRA = for | | | | | | | |
| 13 | 93564_at | Cluster Incl AW122542: UI-M-BH2 2-aox-c-12-0-UI s1 Mus musculus cDNA, 3 end/clone = UI-M-BH2 2-aox-c-12-0-UI/ | 583 | P | 762.3 | P | 640.1 | P | 728.5 |
| | | clone_end = 3/gb = AW122542/gi = 6098066/ug = Mm 20353/len = 471/STRA = for | | | | | | | |
| 13 | 93595_at | Cluster Incl AF111172: Mus musculus lysosomal pepstatin insensitive protease precursor (Cln2) mRNA, complete cds/ | 591 | P | 787.4 | P | 785.7 | P | 514.4 |
| | | cds = (28,1716)/gb = AF111172/gi = 4106912/ug = Mm 20837/len = 2270/STRA = for | | | | | | | |
| 13 | 93599_at | Cluster Incl AI841260: UI-M-AM0-adu-d-05-0-UI s1 Mus musculus cDNA, 3 end/clone = UI-M-AM0-adu-d-05-0-UI/ | 894.6 | P | 980.7 | P | 1250.8 | P | 1067.3 |
| | | clone_end = 3/gb = AI841260/gi = 5475478/ug = Mm 20877/len = 510/STRA = for | | | | | | | |
| 13 | 93815_at | Cluster Incl AI839425: UI-M-AN0-aco-e-06-0-UI s1 Mus musculus cDNA, 3 end/clone = UI-M-AN0-aco-e-06-0-UI/ | 1470.8 | P | 1302.2 | P | 1240.9 | P | 1386.1 |
| | | clone_end = 3/gb = AI839425/gi = 5473638/uq = Mm 21501/len = 402/STRA = for | | | | | | | |
| 13 | 93833_s_at | Cluster Incl X05862: Mouse H2B and H2A histone genes (291A)/cds = (0,380)/gb = X05862/gi = 51302/ug = Mm 21579/ | 528.7 | P | 364.6 | P | 410.2 | P | 390.4 |
| | | len = 381/STRA = for | | | | | | | |
| 13 | 93982_at | Cluster Incl AW122476: UI-M-BH2.2-aou-e-01-0-UI s1 Mus musculus cDNA, 3 end/clone = UI-M-BH2 2-aou-e-01-0-UI/ | 732.9 | P | 689.2 | P | 617.3 | P | 621.9 |
| | | clone_end = 3/gb = AW122476/gi = 6097997/ug = Mm 21707/len = 182/STRA = for | | | | | | | |
| 13 | 94005_at | Cluster Incl AI157548: ue55h05 r1 Mus musculus cDNA, 5 end/clone = IMAGE-1495065/clone_end = 5/gb = AI157548/ | 290.1 | P | 274.7 | P | 354.8 | P | 274.2 |
| | | gi = 3686017/ug = Mm 21780/len = 625/STRA = for | | | | | | | |
| 13 | 94017_s_at | Cluster Incl X98511 M musculus PR264 gene/cds = (0,365)/gb = X98511/gi = 1405746/ug = Mm 21841/len = 366/STRA = for | 4280.6 | P | 4070.7 | P | 4314.6 | P | 3888 |
| 13 | 94040_at | Cluster Incl D73368: House mouse; musculus domesticus adult thymus mRNA for Mer protein, complete cds/cds = (41,355)/ | 3107.3 | P | 2801 | P | 2261.9 | P | 2866.4 |
| | | gb = D73368/gi = 1620873/ug = Mm 21952/len = 754/STRA = for | | | | | | | |
| 13 | 94076_i_at | Cluster Incl D31717 Mouse MARib mRNA for ribophorin, complete cds/cds = (41,1864)/gb = D31717/gi = 1468960/ | 761.2 | P | 945.5 | P | 913.2 | P | 822.7 |
| | | ug = Mm 22130/len = 2192/STRA = for | | | | | | | |
| 13 | 94077_f_at | Cluster Incl D31717 Mouse MARib mRNA for ribophorin, complete cds/cds = (41,1864)/gb = D31717/gi = 1468960/ | 1406.8 | P | 1595.8 | P | 1460.8 | P | 1286.3 |
| | | ug = Mm 22130/len = 2192/STRA = for | | | | | | | |
| 13 | 94209_g_at | Cluster Incl AW045202 UI-M-BH1-alh-08-0-UI.s1 Mus musculus cDNA, 3 end/clone = UI-M-BH1-alh-c-08-0-UI/ | 570.8 | P | 450.7 | P | 485.1 | P | 469.6 |
| | | clone_end = 3/gb = AW045202/gi = 5905731/ug = Mm 2220/len = 430/STRA = for | | | | | | | |
| 13 | 94259_at | Cluster Incl AB024935 Mus musculus Sid3177 mRNA, complete cds/cds = (111,593)/gb = AB024935/gi = 5931546/ | 1111.4 | P | 1026.7 | P | 892.7 | P | 988 |
| | | ug = Mm 22421/len = 1510/STRA = for | | | | | | | |
| 13 | 94273_at | Cluster Incl AI849067: UI-M-AH1-agw-d-04-0-UI s1 Mus musculus cDNA, 3 end/clone = UI-M-AH1-agw-d-04-0-UI/ | 752.5 | P | 801.2 | P | 737.1 | P | 730.2 |
| | | clone_end = 3/gb = AI849067/gi = 5492973/ug = Mm 22485/len = 507/STRA = for | | | | | | | |
| 13 | 94292_at | Cluster Incl AF096285 Mus musculus serine-threonine kinase receptor-associated protein mRNA, complete cds/ | 633.7 | P | 648 | P | 680 | P | 617.2 |
| | | cds = (85,1140)/gb = AF096285/gi = 4063382/ug = Mm.22584/len = 1630/STRA = for | | | | | | | |
| 13 | 94323_at | Cluster Incl AW048132 UI-M-BH1-alq-f-07-0-UI s1 Mus musculus cDNA, 3 end/clone = UI-M-BH1-alq-f-07-0-UI/ | 1744.3 | P | 1763.4 | P | 1435.9 | P | 1572.2 |
| | | clone_end = 3/gb = AW048132/gi = 5908661/ug = Mm 22667/len = 271/STRA = for | | | | | | | |
| 13 | 95404_at | Cluster Incl AW123453 UI-M-BH2 1-apj-e-11-0-UI.s1 Mus musculus cDNA, 3 end/clone = UI-M-BH2 1-apj-e-11-0-UI/ | 505.3 | P | 465.3 | P | 412.5 | P | 391 |
| | | clone_end = 3/gb = AW123453/gi = 6098983/ug = Mm 24199/len = 274/STRA = for | | | | | | | |
| 13 | 95445_at | Cluster Incl AW124194 UI-M-BH2 1-aph-b-0I-0-UI s1 Mus musculus cDNA, 3 end/clone = UI-M-BH2.1-aph-b-01-0-UI/ | 508.2 | P | 483.5 | P | 556.1 | P | 379.7 |
| | | clone_end = 3/gb = AW124194/gi = 6099724/ug = Mm 24593/len = 341/STRA = for | | | | | | | |
| 13 | 95473_s_at | Cluster Incl AW123338 UI-M-BH2 1-apf-c-12-0-UI s1 Mus musculus cDNA, 3 end/clone = UI-M-BH2 1-apl-c-12-0-UI/ | 877 | P | 1044.7 | P | 894.2 | P | 1041.1 |
| | | clone_end = 3/gb = AW123338/gi = 6098868/ug = Mm.24808/len = 425/STRA = for | | | | | | | |
| 13 | 95491_at | Cluster Incl AB015652 Mus musculus gene for DJ-1, complete cds/cds = (0,521)/gb = AB015652/gi = 3256342/ug = Mm 2507/ | 975.9 | P | 1252.1 | P | 1152.8 | P | 969 |
| | | len = 522/STRA = for | | | | | | | |
| 13 | 96016_at | Cluster Incl AW045665 UI-M-BH1-alg-h-11-0-UI.s1 Mus musculus cDNA, 3 end/clone = UI-M-BH1-alg-h-11-0-UI/ | 686.1 | P | 705.7 | P | 681.8 | P | 503.2 |
| | | clone_end = 3/gb = AW045665/gi = 5906194/ug = Mm.25590/len = 395/STRA = for | | | | | | | |
| 13 | 96024_at | Cluster Incl L32838: Mus musculus (clone C7/B9) S-adenosyl homocysteine hydrolase (ahcy) mRNA, complete cds/ | 934.5 | P | 815.6 | P | 678 | P | 813.1 |
| | | cds = (5,1303)/gb = L32836/gi = 904131/ug = Mm.2573/len = 2057/STRA = for | | | | | | | |
| 13 | 96025_g_at | Cluster Incl L32836: Mus musculus (clone C7/B9) S-adenosyl homocysteine hydrolase (ahcy) mRNA, complete cds/ | 927.5 | P | 1124.8 | P | 865.5 | P | 1054.8 |
| | | cds = (5,1303)/gb = L32836/gi = 904131/ug = Mm.2573/len = 2057/STRA = for | | | | | | | |
| 13 | 96632_at | Cluster Incl AB025049: Mus musculus mRNA for Sid393p, complete cds/cds = (96,962)/gb = AB025049/gi = 5931552/ | 2525.5 | P | 2583.9 | P | 2611.9 | P | 2366.4 |
| | | ug = Mm.27218/len = 1621/STRA = for | | | | | | | |
| 13 | 96635_at | Cluster Incl AW125392 UI-M-BH2 3-aqh-c-08-0-UI s1 Mus musculus cDNA, 3 end/clone = UI-M-BH2 3-aqh-c-08-0-UI/ | 205.3 | P | 240.5 | P | 166.3 | P | 231.2 |
| | | clone_end = 3/gb = AW125392/gi = 6100922/ug = Mm 27235/len = 463/STRA = for | | | | | | | |
| 13 | 96696_at | Cluster Incl AI837110 UI-M-AK0-adc-e-02-0-UI s1 Mus musculus cDNA, 3 end/clone = UI-M-AK0-adc-e-02-0-UI/ | 1582.1 | P | 1801.5 | P | 1391 | P | 1684 |
| | | clone_end = 3/gb = AI837110/gi = 5471323/ug = Mm.27545/len = 124/STRA = for | | | | | | | |
| 13 | 97247_at | Cluster Incl AW124778 UI-M-BH2.1-aqa-g-10-0-UI s1 Mus musculus cDNA, 3 end/clone = UI-M-BH2 1-aqa-g-10-0-UI/ | 810.5 | P | 618.2 | P | 776.6 | P | 616.8 |
| | | clone_end = 3/gb = AW124778/gi = 6100308/ug = Mm 27847/len = 462/STRA = for | | | | | | | |
| 13 | 97250_at | Cluster Incl AW049326: UI-M-BH1-amr-f-01-0-UI s1 Mus musculus cDNA, 3 end/clone = UI-M-BH1-amr-f-01-0-UI/ | 1478.9 | P | 1320.8 | P | 1485.1 | P | 1252.5 |
| | | clone_end = 3/gb = AW049326/gi = 5909855/ug = Mm 27858/len = 234/STRA = for | | | | | | | |
| 13 | 97309_at | Cluster Incl AW214164 uo41a02.x1 Mus musculus cDNA, 3 end/clone = IMAGE-2645066/clone_end = 3/gb = AW214164/ | 554.6 | P | 407.4 | P | 544.5 | P | 404.2 |
| | | gi = 6520606/ug = Mm 28066/len = 253/STRA = for | | | | | | | |
| 13 | 97311_at | Cluster Incl AIB36509: UI-M-AI0-aal-f-06-0-UI.s1 Mus musculus cDNA, 3 end/clone = UI-M-AI0-aal-f-06-0-UI/clone_end = 3/ | 1560 | P | 1541.5 | P | 1819 | P | 1589.9 |
| | | gb = AI836509/gi = 5470722/ug = Mm 28077/len = 320/STRA = for | | | | | | | |
| 13 | 97809_at | Cluster Incl AF109906 Mus musculus MHC class III region RD gene, partial cds, Bf, C2, G9A, NG22, G9, HSP70, HSP70, | 980.5 | P | 1005.5 | P | 1030.5 | P | 890.4 |
| | | HSC70t, and smRNP genes, complete cds, G7A gene, partial cds, and unknown genes/cds = (0,3002)/gb = AF109906/ | | | | | | | |
| | | gi = 3986763/ug = Mm 28155/len = 300/STRA = for | | | | | | | |
| 13 | 97824_at | Cluster Incl AW121031 UI-M-BH2 3-aof-b-04-0-UI s1 Mus musculus cDNA, 3 end/clone = UI-M-BH2 3-aof-b-04-0-UI/ | 1806.5 | P | 1763 | P | 1593 | P | 1970.9 |
| | | clone_end = 3/gb = AW121031/gi = 6096364/ug = Mm 28203/len = 396/STRA = for | | | | | | | |
| 13 | 97836_at | Cluster Incl AI843444 UI-M-AQ1-aec-d-07-0-UI.s1 Mus musculus cDNA, 3 end/clone = UI-M-AQ1-aec-d-07-0-UI/ | 812.5 | P | 843.4 | P | 775.4 | P | 770.8 |
| | | clone_end = 3/gb = AI843444/gi = 5477657/ug = Mm 28235/len = 315/STRA = for | | | | | | | |
| 13 | 97907_at | Cluster Incl AW049564 UI-M-BH1-anf-g-07-0-UI s1 Mus musculus cDNA, 3 end/clone = UI-M-BH1-anf-g-07-0-UI/ | 585.8 | P | 645.1 | P | 539.5 | P | 649.1 |
| | | clone_end = 3/gb = AW049564/gi = 5910093/ug = Mm 28470/len = 426/STRA = for | | | | | | | |
| 13 | 98904_at | Cluster Incl AW061339 UI-M-BH1-anw-g-01-0-UI s1 Mus musculus cDNA, 3 end/clone = UI-M-BH1-anw-g-01-0-UI/ | 439.5 | P | 543.3 | P | 452.4 | P | 417.9 |
| | | clone_end = 3/gb = AW061339/gi = 6009090/ug = Mm 28579/len = 376/STRA = for | | | | | | | |
| 13 | 98928_at | Cluster Incl AW122820 UI-M-BH2 1-aoz-b-12-0-UI s1 Mus musculus cDNA, 3 end/clone = UI-M-BH2 1-aoz-b-12-0-UI/ | 545.2 | P | 583.7 | P | 559.8 | P | 412 |
| | | clone_end = 3/gb = AW122820/gi = 6098350/ug = Mm 28652/len = 302/STRA = for | | | | | | | |
| 13 | 98930_at | Cluster Incl AI844701: UI-M-AL1-ahr-h-03-0-UI s1 Mus musculus cDNA, 3 end/clone = UI-M-AL1-ahr-h-03-0-UI/ | 745.5 | P | 681.1 | P | 684.4 | P | 467.3 |
| | | clone_end = 3/gb = AI844701/gi = 5488607/ug = Mm 28668/len = 487/STRA = for | | | | | | | |
| 13 | 94455_at | Cluster Incl AA795062 vs09d03 r1 Mus musculus cDNA, 5 end/clone = IMAGE-1137701/clone_end = 5/gb = AA795062/ | 910.1 | P | 1007.4 | P | 848.1 | P | 876.7 |
| | | gi = 2858017/ug = Mm 28803/len = 536/STRA = for | | | | | | | |
| 13 | 94503_at | Cluster Incl AI842492 UI-M-AM1-afy-h-05-0-UI s1 Mus musculus cDNA, 3 end/clone = UI-M-AM1-afy-h-05-0-UI/ | 578 | P | 628.5 | P | 691.8 | P | 667.3 |
| | | clone_end = 3/gb = AI842492/gi = 5476705/ug = Mm 28952/len = 259/STRA = for | | | | | | | |
| 13 | 94510_at | Cluster Incl AI853476 UI-M-BH0-ajy-h-09-0-UI s1 Mus musculus cDNA, 3 end/clone = UI-M-BH0-ajy-h-09-0-UI/ | 538.6 | P | 481.8 | P | 456.7 | P | 522.5 |
| | | clone_end = 3/gb = AI853476/gi = 5497382/ug = Mm 28975/len = 537/STRA = for | | | | | | | |
| 13 | 94524_at | Cluster Incl AW123469 UI-M-BH2 1-apj-g-04-0-UI.s1 Mus musculus cDNA, 3 end/clone = UI-M-BH2.1-apj-g-04-0-UI/ | 367.1 | P | 352.5 | P | 327.4 | P | 347.1 |
| | | clone_end = 3/gb = AW123469/gi = 6098999/ug = Mm 29028/len = 452/STRA = for | | | | | | | |
| 13 | 94532_at | Cluster Incl U13841.ATPase, H+ transporting lysosomal (vacuolar proton pump) 32 kDa/cds = (0,686)/gb = U13841/ | 887 | P | 944 | P | 883.8 | P | 885.9 |
| | | gi = 1184662/ug = Mm.29045/len = 687/STRA = for | | | | | | | |
| 13 | 95049_at | Cluster Incl AI837853 UI-M-AP0-abk-d-04-0-UI s1 Mus musculus cDNA, 3 end/clone = UI-M-AP0-abk-d-04-0-UI/ | 3209.7 | P | 3270.2 | P | 2586.4 | P | 3123.9 |
| | | clone_end = 3/gb = AI837853/gi = 5472066/ug = Mm 29135/len = 429/STRA = for | | | | | | | |
| 13 | 95067_at | Cluster Incl AI837821 UI-M-AK0-adl-h-06-0-UI s1 Mus musculus cDNA, 3 and/clone = UI-M-AK0-adl-h-06-0-UI/ | 308.3 | P | 366.9 | P | 335.5 | P | 278.2 |
| | | clone_end = 3/gb = AI837821/gi = 5472034/ug = Mm 29183/len = 468/STRA = for | | | | | | | |
| 13 | 95734_at | Cluster Incl AI852230 UI-M-BH0-ajc-f-07-0-UI s1 Mus musculus cDNA, 3 end/clone = UI-M-BH0-ajc-f-07-0-UI/clone_end = 3/ | 672.4 | P | 620.2 | P | 575.8 | P | 660.6 |
| | | gb = AI852230/gi = 5496136/ug = Mm 29746/len = 392/STRA = for | | | | | | | |
| 13 | 95743_at | Cluster Incl AI846078 UI-M-AN1-afj-a-03-0-UI s2 Mus musculus cDNA, 3 end/clone = UI-M-AN1-afj-a-03-0-UI/ | 1606.9 | P | 1115.8 | P | 1152.8 | P | 1119.3 |
| | | clone_end = 3/gb = AI846078/gi = 5490024/ug = Mm.29769/len = 335/STRA = for | | | | | | | |
| 13 | 96242_at | Cluster Incl AW125751 UI-M-BH2 2-aqi-h-05-0-UI s1 Mus musculus cDNA, 3 end/clone = UI-M-BH2.2-aqi-h-05-0-UI/ | 331.3 | P | 359.4 | P | 288.7 | P | 313.5 |
| | | clone_end = 3/gb = AW125751/gi = 6101246/ug = Mm 29802/len = 240/STRA = for | | | | | | | |
| 13 | 96261 _at | Cluster Incl AW122694 UI-M-BH2.2-aot-b-04-0-UI s1 Mus musculus cDNA, 3 end/clone = UI-M-BH2 2-aot-b-04-0-UI/ | 1463.4 | P | 1370.6 | P | 1111.3 | P | 1099.7 |
| | | clone_end = 3/gb = AW122694/gi = 6098224/ug = Mm 29828/len = 456/STRA = for | | | | | | | |
| 13 | 96280_at | Cluster Incl AI851603 UI-M-BH0-aln-a-07-0-UI.s1 Mus musculus cDNA, 3 end/clone = UI-M-BH0-aln-a-07-0-UI/ | 923.6 | P | 1357 | P | 1141.4 | P | 1181.2 |
| | | clone_end = 3/gb = AI851603/gi = 5495509/ug = Mm.29867/len = 415/STRA = for | | | | | | | |
| 13 | 96295_at | Cluster Incl AW122030 UI-M-BH2 2-aoo-b-05-0-UI.s1 Mus musculus cDNA, 3 end/clone = UI-M-BH2.2-aoo-b-05-0-UI/ | 768.6 | P | 1917.3 | P | 1413 | P | 1983 |
| | | clone_end = 3/gb = AW122030/gi = 6097436/ug = Mm 29902/len = 358/STRA = for | | | | | | | |
| 13 | 96318_at | Cluster Incl AW045739-UI-M-BH1-akq-g-08-0-UI s1 Mus musculus cDNA, 3 end/clone = UI-M-BH1-akq-g-08-0-UI/ | 528 | P | 486.1 | P | 465.4 | P | 563.2 |
| | | clone_end = 3/gb = AW045739/gi = 5906268/ug = Mm 29925/len = 263/STRA = for | | | | | | | |
| 13 | 96352_at | Cluster Incl AI848845 UI-M-AJ1-ahb-d-02-0-UI.s1 Mus musculus cDNA, 3 end/clone = UI-M-AJ1-ahb-d-02-0-UI/ | 871.8 | P | 997.7 | P | 680 | P | 909.7 |
| | | clone_end = 3/gb = AI848845/gi = 5492751/ug = Mm 30003/len = 402/STRA = for | | | | | | | |
| 13 | 96847_at | Cluster Incl AI845393 UI-M-AO1-aej-a-03-0-UI s1 Mus musculus cDNA, 3 end/clone = UI-M-AO1-aoj-a-03-0-UI/ | 807.5 | P | 1076.7 | P | 738.5 | P | 668.8 |
| | | clone_end = 3/gb = AI845393/gi = 5489299/ug = Mm 30028/len = 278/STRA = for | | | | | | | |
| 13 | 96864_at | Cluster Incl AI848770: UI-M-AJ1-ahg-a-08-0-UI s1 Mus musculus cDNA, 3 end/clone = UI-M-AJ1-ahg-a-08-0-UI/ | 358.3 | P | 399.5 | P | 338.8 | P | 335.9 |
| | | clone_end = 3/gb = AI848770/gi = 5492676/ug = Mm 30057/len = 383/STRA = for | | | | | | | |
| 13 | 96959_at | Cluster Incl AW210080: ul50f12.y1 Mus musculus cDNA, 5 end/clone = IMAGE-2101775/clone_end = 5/gb = AW210080/ | 1591.7 | P | 1719.5 | P | 1571 | P | 1716.5 |
| | | gi = 6516020/ug = Mm 30233/len = 505/STRA = for | | | | | | | |
| 13 | 97458_at | Cluster Incl AI845935: UI-M-AK1-aex-f-04-0-UI s1 Mus musculus cDNA, 3 end/clone = UI-M-AK1-aex-f-04-0-UI/ | 1162.7 | P | 1338.9 | P | 1083.2 | P | 1082.8 |
| | | clone_end = 3/gb = AI845935/gi = 5489841/ug = Mm 30267/len = 380/STRA = for | | | | | | | |
| 13 | 97472_at | Cluster Incl AJ006341 Mus musculus mRNA for peroxisomal integral membrane protein PMP34/cds = (47,970)/ | 668.9 | P | 725.8 | P | 856.7 | P | 868.6 |
| | | gb = AJ006341/gi = 3183980/ug = Mm 306/1en = 1700/STRA = for | | | | | | | |
| 13 | 97477_at | Cluster Incl AW124594: UI-M-BH2.1-apt-g-02-0-UI.s1 Mus musculus cDNA, 3 end/clone = UI-M-BH2.1-apt-g-02-0-UI/ | 624.6 | P | 506.9 | P | 866.2 | P | 615.4 |
| | | clone_end = 3/gb = AW124594/gi = 6100124/ug = Mm.30718/len = 407/STRA = for | | | | | | | |
| 13 | 97479_at | Cluster Incl AJ130961: Mus musculus mRNA for ubiquitin-conjugating enzyme/cds = (20,484)/gb = AJ130961/gi = 3928492/ | 996 | P | 1289.8 | P | 1001.1 | P | 955.7 |
| | | ug = Mm 3074/len = 2597/STRA = for | | | | | | | |
| 13 | 97535_at | Cluster Incl D87661: Mus musculus mRNA for 14-3-3 eta, complete cds/cds = (184,924)/gb = D87661/gi = 1526540/ | 876.7 | P | 899.4 | P | 1064.5 | P | 879.3 |
| | | ug = Mm 3308/len = 1098/STRA = for | | | | | | | |
| 13 | 98037_at | Cluster Incl AW122782 UI-M-BH2 1-aoy-d-05-0-UI s1 Mus musculus cDNA, 3 end/clone = UI-M-BH2 1-aoy-d-05-0-UI/ | 590.6 | P | 562.3 | P | 597.8 | P | 618.5 |
| | | clone_end = 3/gb = AW122782/gi = 6098312/ug = Mm 33907/len = 153/STRA = for | | | | | | | |
| 13 | 98107_at | Cluster Incl AW123801 UI-M-BH2 1-apm-e-08-0-UI s1 Mus musculus cDNA, 3 end/clone = UI-M-BH2 1-apm-e-08-0-UI/ | 1103 | P | 1053.8 | P | 1056.3 | P | 1346.8 |
| | | clone_end = 3/gb = AW123801/gi = 6099331/ug = Mm.34796/len = 367/STRA = for | | | | | | | |
| 13 | 98129_at | Cluster Incl AI852553 UI-M-BH0-aiw-g-02-0-UI s1 Mus musculus cDNA, 3 end/clone = UI-M-BH0-aiw-g-02-0-UI/ | 3465.3 | P | 3893 | P | 3576.9 | P | 3606.1 |
| | | clone_end = 3/gb = AI852553/gi = 5496459/ug = Mm 3532/len = 276/STRA = for | | | | | | | |
| 13 | 98533_at | Cluster Incl AI854779 UI-M-BH0-aka-d-08-0-UI s1 Mus musculus cDNA, 3 end/clone = UI-M-BH0-aka-d-08-0-UI/ | 1291.7 | P | 1401.9 | P | 1470 | P | 1049.6 |
| | | clone_end = 3/gb = AI854779/gi = 5498685/ug = Mm 35849/len = 428/STRA = for | | | | | | | |
| 13 | 98574_at | Cluster Incl AI835685 UI-M-AJ0-aau-g-09-0-UI s1 Mus musculus cDNA, 3 end/clone = UI-M-AJ0-aau-g-09-0-UI/ | 665 | P | 704.3 | P | 679.6 | P | 631.5 |
| | | clone_end = 3/gb = AI835685/gi = 5469898/ug = Mm 3757/len = 227/STRA = for | | | | | | | |
| 13 | 98609_at | Cluster Incl AJ250723 Mus musculus mRNA for septin-like protein Sint1 (Sint1 gene)/cds = (177,1181)/gb = AJ250723/ | 1436 | P | 1597.1 | P | 1564.7 | P | 1469.6 |
| | | gi = 6165418/ug = Mm 38450/len = 2942/STRA = for | | | | | | | |
| 13 | 99106_at | Cluster Incl AF071315 Mus musculus COP9 complex subunit 6 (COPS6) mRNA, complete cds/cds = (48,1022)/ | 1341.3 | P | 1721 | P | 1430.6 | P | 1427.8 |
| | | gb = AF071315/gi = 3309171/ug = Mm 3981/len = 1160/STRA = for | | | | | | | |
| 13 | 99113_at | Cluster Incl AF071313 Mus musculus COP9 complex subunit 3 (COPS3) mRNA, complete cds/cds = (43,1314)/ | 547.3 | P | 625.2 | P | 508 | P | 480.6 |
| | | gb = AF071313/gi = 3309167/ug = Mm 40/len = 1583/STRA = for | | | | | | | |
| 13 | 99593_at | Cluster Incl AI852355: UI-M-BH0-ajo-c-05-0-UI s1 Mus musculus cDNA, 3 end/clone = UI-M-BH0-ajo-c-05-0-UI/ | 1574.5 | P | 1690.4 | P | 1422.5 | P | 1223.7 |
| | | clone_end = 3/gb = AI852355/gi = 5496261/ug = Mm 42805/len = 413/STRA = for | | | | | | | |
| 13 | 99596_f_at | Cluster Incl M13963 Guanine nucleotide binding protein, alpha inhibiting 2/cds = (97,1164)/gb = M13963/gi = 193513/ | 5190.8 | P | 5647.4 | P | 5744.1 | P | 5589.7 |
| | | ug = Mm 4284/len = 1316/STRA = for | | | | | | | |
| 13 | 99598_g_at | Cluster Incl AI841629 UI-M-AO0-acg-c-01-0-UI s1 Mus musculus cDNA, 3 end/clone = UI-M-AO0-acg-c-01-0-UI/ | 2042.7 | P | 2375.4 | P | 2606 | P | 2385.3 |
| | | clone_end = 3/gb = AI841629/gi = 5475842/ug = Mm 4284/len = 436/STRA = for | | | | | | | |
| 13 | 99618_at | Cluster Incl AI853523: UI-M-BH0-ajd-e-03-0-UI s1 Mus musculus cDNA, 3 end/clone = UI-M-BH0-ajd-e-03-0-UI/ | 1378.9 | P | 1545 | P | 998.7 | P | 1290.4 |
| | | clone_end = 3/gb = AI853523/gi = 5497429/ug = Mm 43162/len = 425/STRA = for | | | | | | | |
| 13 | 99619_at | Cluster Incl AIB47483 UI-M-AI1-afs-g-03-0-UI s1 Mus musculus cDNA, 3 end/clone = UI-M-AI1-afs-g-03-0-UI/clone_end = 3/ | 378.7 | P | −40 | P | 220.4 | P | 288 |
| | | gb = AI847483/gi = 5491389/ug = Mm 43212/len = 471/STRA = for | | | | | | | |
| 13 | 99637_at | Cluster Incl AF011450 Procollagen, type XV/cds = (158,4259)/gb = AF011450/gi = 2558824/ug = Mm 4352/len = 5176/ | 1011.2 | P | 783.2 | P | 1191.8 | P | 849.9 |
| | | STRA = for | | | | | | | |
| 13 | 99655_at | Cluster Incl AB025405 Mus musculus mRNA for sid2895p, complete cds/cds = (98,637)/gb = AB025405/gi = 5931558/ | 937.4 | P | 998.8 | P | 951.1 | P | 974.4 |
| | | ug = Mm 43760/len = 1113/STRA = for | | | | | | | |
| 13 | 100040_at | Cluster Incl AI843081: UI-M-AK1-aes-b-06-0-UI s1 Mus musculus cDNA, 3 end/clone = UI-M-AK1-aes-b-06-0-UI/ | 179.4 | P | 182 | P | 249.5 | P | 284.1 |
| | | clone_end = 3/gb = AI843081/gi = 5477294/ug = Mm 44225/len = 232/STRA = for | | | | | | | |
| 13 | 100050_at | Cluster Incl M31885 Inhibitor of DNA binding 1/cds = (0,532)/gb = M31885/gi = 194090/ug = Mm 444/len = 927/STRA = for | 1305.6 | P | 927.7 | P | 954 | P | 1363.3 |
| 13 | 100062_at | Cluster Incl X62154.Mini chromosome maintenance deficient (S cerevisiae)/cds = (0,2387)/gb = X62154/gi = 53550/ | 380.8 | P | 494.7 | P | 295.9 | P | 338.1 |
| | | ug = Mm 4502/len = 2813/STRA = rev | | | | | | | |
| 13 | 100065_r_at | Cluster Incl M63801 Mus musculus connexin 43 (alpha-1 gap junction) mRNA, complete cds/cds = (220,1368)/gb = M63801/ | 3224.6 | P | 2907.1 | P | 2554.2 | P | 2606.7 |
| | | gi = 191773/ug = Mm 4504/len = 1408/STRA = for | | | | | | | |
| 13 | 100578_at | Cluster Incl M33934 Mouse IMP dehydrogenase mRNA, complete cds/cds = (66,1610)/gb = M33934/gi = 198393/ | 1320.2 | P | 1350.8 | P | 1168.9 | P | 1515.5 |
| | | ug = Mm 6065/len = 1707/STRA = for | | | | | | | |
| 13 | 100628_at | Cluster Incl AI840263 UI-M-AH0-act-e-04-0-UI.s1 Mus musculus cDNA, 3 end/clone = UI-M-AH0-act-e-04-0-UI/ | 1004.6 | P | 1120.9 | P | 918.5 | P | 856.1 |
| | | clone_end = 3/gb = AI840263/gi = 5474476/ug = Mm 666/len = 248/STRA = for | | | | | | | |
| 13 | 101017_at | Cluster Incl AA791962: vs65h09 r1 Mus musculus cDNA, 5 end/clone = IMAGE-1151201/clone_end = 5/gb = AA791962/ | 680.7 | P | 558.1 | P | 548.7 | P | 579.4 |
| | | gi = 2854917/ug = Mm 6839/len = 336/STRA = rev | | | | | | | |
| 13 | 101061_at | Cluster Incl AI845293 UI-M-BG0-ahw-f-04-0-UI.s1 Mus musculus cDNA, 3 end/clone = UI-M-BG0-ahw-f-04-0-UI/ | 1247.8 | P | 1467 | P | 1430.9 | P | 1477.6 |
| | | clone_end = 3/gb = AI845293/gi = 5489199/ug = Mm 7091/len = 254/STRA = for | | | | | | | |
| 13 | 101081_at | Cluster Incl AJ010483 Mus musculus mRNA for CtBP1 protein/cds = (99,1421)/gb = AJ010483/gi = 3452506/ug = Mm 7286/ | 1252.9 | P | 1406.2 | P | 1375.3 | P | 1301.7 |
| | | len = 2240/STRA = for | | | | | | | |
| 13 | 101104_at | Cluster Incl AB001990: Mus musculus mRNA for Dcra, complete cds/cds = (50,943)/gb = AB001990/gi = 2588992/ | 531.7 | P | 604 | P | 558.6 | P | 587.1 |
| | | ug = Mm 7472/len = 2157/STRA = for | | | | | | | |
| 13 | 101105_at | Cluster Incl AB025349: Mus musculus L2BP1/BAF mRNA for LAP2 binding protein 1, complete cds/cds = (57,326)/ | 1606.2 | P | 1523.6 | P | 1517.2 | P | 1546 |
| | | gb = AB025349/gi = 5708070/ug = Mm 7508/len = 603/STRA = for | | | | | | | |
| 13 | 101519_at | Cluster Incl M29264 Mouse signal recognition particle subunit (SRP14) mRNA, complete cds/cds = (7,339)/gb = M29264/ | 1261.3 | P | 1179.7 | P | 1152.7 | P | 1133.9 |
| | | gi = 201062/ug = Mm.850/len = 706/STRA = for | | | | | | | |
| 13 | 101521_at | Cluster Incl AB013819 Mus musculus mRNA for TIAP, complete cds/cds = (108,530)/gb = AB013819/gi = 3135206/ | 544.9 | P | 595.3 | P | 530 | P | 411.8 |
| | | ug = Mm.8552/len = 955/STRA = for | | | | | | | |
| 13 | 101964_at | Cluster Incl U05809: Transketolase/cds = (91,1962)/gb = U05809/gi = 452485/ug = Mm.9307/len = 2062/STRA = for | 996.1 | P | 1368.5 | P | 1135.9 | P | 1236.9 |
| 13 | 103364_f_at | Cluster Incl AW045256 UI-M-BH1-akn-a-02-0-UI.s1 Mus musculus cDNA, 3 end/clone = UI-M-BH1-akn-a-02-0-UI/ | 442.1 | P | 410 | P | 400.2 | P | 296.6 |
| | | clone_end = 3/gb = AW045256/gi = 5905785/ug = Mm.18520/len = 324/STRA = for | | | | | | | |
| 13 | 103381_at | Cluster Incl AI838735 UI-M-AO0-acf-c-11-0-UI s1 Mus musculus cDNA, 3 end/clone = UI-M-AO0-acf-c-11-0-UI/ | 753.1 | P | 766.1 | P | 885.6 | P | 747.9 |
| | | clone_end = 3/gb = AI838735/gi = 5472948/ug = Mm.18634/len = 413/STRA = for | | | | | | | |
| 13 | 103606_r_at | Cluster Incl AW121438 UI-M-BH2 2-aon-c-10-0-UI.s1 Mus musculus cDNA, 3 end/clone = UI-M-BH2 2-aon-c-10-0-UI/ | 390.6 | P | 429.8 | P | 392.7 | P | 416.7 |
| | | clone_end = 3/gb = AW121438/gi = 6096771/ug = Mm.20156/len = 269/STRA = for | | | | | | | |
| 13 | 103620_a_at | Cluster Incl U77714: Survival motor neuron/cds = (0,866)/gb = U77714/gi = 1946332/ug = Mm 2025/len = 1250/STRA = for | 377.3 | P | 447.4 | P | 459.7 | P | 416.8 |
| 13 | 103835_f_at | Cluster Incl AF085192: Mus musculus neural visinin-like protein 3 (NVP-3) mRNA, complete cds/cds = (260,841)/ | 925.2 | P | 719.1 | P | 808 | P | 829.3 |
| | | gb = AF085192/gi = 3600082/ug = Mm.20937/len = 1565/STRA = for | | | | | | | |
| 13 | 104108_at | Cluster Incl AJ245569: Mus musculus partial mRNA for hypothetical protein (ORF37 DNA)/cds = (218,4009)/gb = AJ245569/ | 301.2 | P | 287.5 | P | 306.2 | P | 227 |
| | | gi = 5869933/ug = Mm 21904/len = 4907/STRA = for | | | | | | | |
| 13 | 104277_at | Cluster Incl AI834815: UI-M-AM0-ads-a-05-0-UI.s1 Mus musculus cDNA, 3 end/clone = UI-M-AM0-ads-a-05-0-UI/ | 298.4 | P | 322.7 | P | 261.8 | P | 147.6 |
| | | clone_end = 3/gb = AI834815/gi = 5468993/ug = Mm 22218/len = 168/STRA = for | | | | | | | |
| 13 | 104333_at | Cluster Incl U69488 Mus musculus viral envelope like protein (G7e) gene, complete cds/cds = (154,1944)/gb = U69488/ | 580.4 | P | 602.2 | P | 532.4 | P | 534 |
| | | gi = 2988386/ug = Mm 22506/len = 2910/STRA = for | | | | | | | |
| 13 | 96232_at | Cluster Incl AI854281: UI-M-BH0-ajs-d-09-0-UI s1 Mus musculus cDNA, 3 end/clone = UI-M-BH0-ajs-d-09-0-UI/ | 416.2 | P | 348.5 | P | 348 | P | 395.9 |
| | | clone_end = 3/gb = AI854281/gi = 5498187/ug = Mm.28776/len = 440/STRA = for | | | | | | | |
| 13 | 97374_at | Cluster Incl AI840458 UI-M-AN0-aci-c-06-0-UI s1 Mus musculus cDNA, 3 end/clone = UI-M-AN0-aci-c-06-0-UI/ | 441 | P | 476.1 | P | 480.5 | P | 376 .6 |
| | | clone_end = 3/gb = AI840458/gi = 5474671/ug = Mm 29721/len = 430/STRA = for | | | | | | | |
| 13 | 97401_at | Cluster Incl AW124244 UI-M-BH2 1-aph-f-05-0-UI s1 Mus musculus cDNA, 3 end/clone = UI-M-BH2 1-aph-f-05-0-UI/ | 540.5 | P | 450.1 | P | 548.9 | P | 506.9 |
| | | clone_end = 3/gb = AW124244/gi = 6099774/ug = Mm.29878/len = 381/STRA = for | | | | | | | |
| 13 | 97436_at | Cluster Incl AA869653: vq20g06 r1 Mus musculus cDNA, 5 end/clone = IMAGE-1094842/clone_end = 5/gb = AA869653/ | 264.9 | P | 257.9 | P | 127 .7 | P | 120.9 |
| | | gi = 2965098/ug = Mm 30110/len = 240/STRA = for | | | | | | | |
| 13 | 98459_at | Cluster Incl AA913994: vy91d09.r1 Mus musculus cDNA, 5 end/clone = IMAGE-1313585/clone_end = 5/gb = AA913994/ | 306.3 | P | 294.7 | P | 335.9 | P | 349.9 |
| | | gi = 3053386/ug = Mm 34759/len = 511/STRA = for | | | | | | | |
| 13 | 98993_at | Cluster Incl U59418 Mus musculus protein phosphatase 2A B alpha3 regulatory subunit mRNA, partial cds/cds = (406,1671)/ | 834.2 | P | 1061.6 | P | 930.7 | P | 896.7 |
| | | gb = U59418/gi = 2429084/ug = Mm 3785/len = 1796/STRA = for | | | | | | | |
| 13 | 99546_at | Cluster Incl M77831: Mouse binding protein 13 (Fkbp13) gene exons 1-5, complete cds/cds = (155,577)/gb = M77831/ | 887.9 | P | 859.9 | P | 658.9 | P | 738.1 |
| | | gi = 433782/ug = Mm.4234/len = 740/STRA = for | | | | | | | |
| 13 | 100885_at | Cluster Incl AF013166: Mus musculus serine/threonine kinase (nek2) mRNA, complete cds/cds = (57,1388)/gb = AF013166/ | 525.3 | P | 631 | P | 464.6 | P | 526.8 |
| | | gi = 2331225/ug = Mm 5379/len = 2869/STRA = for | | | | | | | |
| 13 | 101385_at | Cluster Incl AW047312 UI-M-BH1-amh-f-09-0-UI.s1 Mus musculus cDNA, 3 end/clone = UI-M-BH1-amh-f-09-0-UI/ | 1281 | P | 1215.8 | P | 946.3 | P | 885.8 |
| | | clone_end = 3/gb = AW047312/gi = 5907841/ug = Mm.7142/len = 493/STRA = for | | | | | | | |
| 13 | 102879_s_at | Cluster Incl M31314 Fc receptor, IgG, high affinity I/cds = (1,1215)/gb = M31314/gi = 200752/ug = Mm.150/len = 1279/ | 741 | P | 671.1 | P | 685.3 | P | 864.2 |
| | | STRA = for | | | | | | | |
| 13 | 103203_f_at | Cluster Incl AW259499 up28d02.x1 Mus musculus cDNA, 3 end/clone = IMAGE-2655651/clone_end = 3/gb = AW259499/ | 239.2 | P | 194.7 | P | 163.4 | P | 249.7 |
| | | gi = 6632417/ug = Mm.1911/len = 454/STRA = for | | | | | | | |
| 13 | 103442_at | Cluster Incl AI843399 UI-M-AQ1-adz-g-11-0-UI s1 Mus musculus cDNA, 3 end/clone = UI-M-AQ1-adz-g-11-0-UI/ | 336.9 | P | 327.8 | P | 368.7 | P | 346.4 |
| | | clone_end = 3/gb = AI843399/gi = 5477612/ug = Mm 21475/len = 471/STRA = for | | | | | | | |
| 13 | 104491_at | Cluster Incl AI509330 mt94e09.y1 Mus musculus cDNA, 5 end/clone = IMAGE-637576/clone_end = 5/gb = AI509330/ | 686.8 | P | 656.6 | P | 592.4 | P | 676.6 |
| | | gi = 4408235/ug = Mm.3028/len = 360/STRA = for | | | | | | | |
| 13 | 92304_at | Cluster Incl D31863 Phosphatidylinositol glycan, class A/cds = (62,1519)/gb = D31863/gi = 1402591/ug = Mm 3781/len = 3534/ | 94.5 | P | 67 | P | 136.5 | P | 129.4 |
| | | STRA = for | | | | | | | |
| 13 | 92428_at | Cluster Incl AF039405: Mus musculus arsenite-translocating ATPase (ArsA) mRNA, complete cds/cds = (0,1052)/ | 631 | P | 517.3 | P | 536.5 | P | 608.5 |
| | | gb = AF039405/gi = 2745699/ug = Mm 41475/len = 1053/STRA = for | | | | | | | |
| 13 | 93670_at | Cluster Incl AW048233 UI-M-BH1-amc-h-10-0-UI s1 Mus musculus cDNA, 3 end/clone = UI-M-BH1-amc-h-10-0-UI/ | 563.9 | P | 567.8 | P | 563.1 | P | 553.8 |
| | | clone_end = 3/gb = AW048233/gi = 5908762/ug = Mm 8068/len = 429/STRA = for | | | | | | | |
| 13 | 94788_f_at | Cluster Incl X04663 Mouse mRNA for beta-tubulin (lsotype Mbeta 5)/cds = (78,1412)/gb = X04663/gi = 55047/ug = Mm 1703/ | 4540.4 | P | 5361.9 | P | 5060.7 | P | 4663.5 |
| | | len = 1591/STRA = for | | | | | | | |
| 13 | 97682_r_at | Cluster Incl J03953 Mouse, glutathione transferase GT9 3 mRNA, 3 end/cds = (0,630)/gb = J03953/gi = 193689/ | 1464.6 | P | 1281.8 | P | 1338.7 | P | 1323.9 |
| | | ug = Mm 37199/len = 1090/STRA = for | | | | | | | |
| 13 | 97758_at | Cluster Incl AB023564 Mus musculus gene for type I peroxiredoxin/cds = (11,610)/gb = AB023564/gi = 6467220/ug = Mm 412/ | 3214.6 | P | 3531.8 | P | 3266.5 | P | 3518.9 |
| | | len = 864/STRA = for | | | | | | | |
| 13 | 95791_s_at | Cluster Incl U14648 Splicing factor, arginine/serine-rich 10/cds = (0,853)/gb = U14648/gi = 555923/ug = Mm 87815/len = 1772/ | 1133.8 | P | 1054.3 | P | 1320 | P | 1032.9 |
| | | STRA = for | | | | | | | |
| 13 | 97055_s_at | Cluster Incl AI118194: ue34e03 x1 Mus musculus cDNA, 3 end/clone = IMAGE-1482268/clone_end = 3/gb = AI118194/ | 4206.3 | P | 4112.9 | P | 4012.7 | P | 4265.7 |
| | | gi = 3518518/ug = Mm 89213/len = 567/STRA = rev | | | | | | | |
| 13 | 97061_g_at | Cluster Incl AW215489: up04f11.x1 Mus musculus cDNA, 3 end/clone = IMAGE-2651085/clone_end = 3/gb = AW215489/ | 4276.1 | P | 4331.3 | P | 3824.8 | P | 4058.2 |
| | | gi = 6526165/ug = Mm 89290/len = 522/STRA = rev | | | | | | | |
| 13 | 99678_f_at | Cluster Incl AU018994: AU018994 Mus musculus cDNA, 3 end/clone = J0511B11/clone_end = 3/gb = AU018994/ | 753.1 | P | 851 | P | 702.4 | P | 754 |
| | | gi = 3374497/ug = Mm 95645/len = 265/STRA = rev | | | | | | | |
| 13 | 102476_f_at | Cluster Incl AW121303: UI-M-BH2 3-aog-g-01-0-UI s1 Mus musculus cDNA, 3 end/clone = UI-M-BH2.3-aog-g-01-0-UI/ | 717.9 | P | 748.6 | P | 727 | P | 612.3 |
| | | clone_end = 3/gb = AW121303/gi = 6096636/ug = Mm48675/len = 217/STRA = rev | | | | | | | |
| 13 | 93016_at | Cluster Incl U56243 Mus musculus testis 14-3-3 protein theta-subtype mRNA, complete cds/cds = (20,757)/gb = U56243/ | 472 | P | 453 | P | 409.7 | A | 472.8 |
| | | gi = 1777958/ug = Mm 14722/len = 761/STRA = for | | | | | | | |
| 13 | 93993_at | Cluster Incl AI851062: UI-M-BH0-ajv-h-01-0-UI s1 Mus musculus cDNA, 3 end/clone = UI-M-BH0-ajv-h-01-0-UI/ | 471.8 | P | 426.9 | P | 380.1 | P | 466.9 |
| | | clone_end = 3/gb = AI851062/gi = 5494968/ug = Mm 21752/len = 400/STRA = for | | | | | | | |
| 13 | 94071_at | Cluster Incl AI847904: UI-M-AI1-afl-a-09-0-UI s1 Mus musculus cDNA, 3 end/clone = UI-M-AI1-afl-a-09-0-UI/clone_end = 3/ | 264.1 | P | 188.1 | P | 190.3 | P | 192.1 |
| | | gb = AI847904/gi = 5491810/ug = Mm.22114/len = 350/STRA = for | | | | | | | |
| 13 | 94882_i_at | Cluster Incl AI850000: UI-M-BG0-aib-e-07-0-UI s1 Mus musculus cDNA, 3 end/clone = UI-M-BG0-aib-e-07-0-UI/ | 857.7 | P | 937.8 | P | 864.9 | P | 879.1 |
| | | clone_end = 3/gb = AI850000/gi = 5493906/ug = Mm 23693/len = 409/STRA = for | | | | | | | |
| 13 | 96081_at | Cluster Incl X60980.Mus musculus TK gene encoding thymidine kinase, exon 1 and 2, partial/cds = (0,97)/gb = X60980/ | 616.4 | P | 513.8 | P | 52.5 | P | 369.3 |
| | | gi = 54809/ug = Mm.2661/len = 98/STRA = for | | | | | | | |
| 13 | 96698_at | Cluster Incl AI835520: UI-M-AI0-aam-d-11-0-UI s1 Mus musculus cDNA, 3 end/clone = UI-M-AI0-aam-d-11-0-UI/ | 662.2 | P | 472.5 | A | 370.8 | P | 486.5 |
| | | clone_end = 3/gb = AI835520/gi = 5469733/ug = Mm.27558/len = 421/STRA = for | | | | | | | |
| 13 | 97295_at | Cluster Incl AW122331: UI-M-BH2 2-aos-f-09-0-UI.s1 Mus musculus cDNA, 3 end/clone = UI-M-BH2.2-aos-f-09-0-UI/ | 514.7 | P | 564.3 | P | 466.8 | P | 345.2 |
| | | clone_end = 3/gb = AW122331/gi = 6097816/ug = Mm 28038/len = 409/STRA = for | | | | | | | |
| 13 | 97884_at | Cluster Incl AI844175 UI-M-AL1-ahj-h-09-0-UI s1 Mus musculus cDNA, 3 end/clone = UI-M-AL1-ahj-h-09-0-UI/ | 216.1 | P | 236.7 | P | 308.2 | P | 355.2 |
| | | clone_end = 3/gb = AI844175/gi = 5488081/ug = Mm 28378/len = 319/STRA = for | | | | | | | |
| 13 | 98887_at | Cluster Incl AW125607: UI-M-BH2.2-aqj-f-06-0-UI.s1 Mus musculus cDNA, 3 end/clone = UI-M-BH2.2-aqj-f-06-0-UI/ | 395.4 | P | 533.6 | P | 493.4 | P | 539.4 |
| | | clone_end = 3/gb = AW125607/gi = 6101137/ug = Mm.28538/len = 461/STRA = for | | | | | | | |
| 13 | 94449_at | Cluster Incl AI854522: UI-M-BH0-ajj-d-05-0-UI.s1 Mus musculus cDNA, 3 end/clone = UI-M-BH0-ajj-d-05-0-UI/clone_end = 3/ | 316.4 | P | 588.1 | P | 589.3 | P | 443.4 |
| | | gb = AI854522/gi = 5498428/ug = Mm 28785/len = 358/STRA = for | | | | | | | |
| 13 | 94453_at | Cluster Incl AI852571 UI-M-BH0-aiw-h-10-0-UI s1 Mus musculus cDNA, 3 end/clone = UI-M-BH0-aiw-h-10-0-UI/ | 233.3 | P | 308.4 | P | 313 | P | 218.2 |
| | | clone_end = 3/gb = AI852571/gi = 5496477/ug = Mm.28794/len = 293/STRA = for | | | | | | | |
| 13 | 96262_at | Cluster Incl AI836812 UI-M-AJ0-abd-b-04-0-UI.s1 Mus musculus cDNA, 3 end/clone = UI-M-AJ0-abd-b-04-0-UI/ | 572.1 | P | 736.9 | P | 667.8 | P | 775.8 |
| | | clone_end = 3/gb = AI836812/gi = 5471025/ug = Mm 29829/len = 289/STRA = for | | | | | | | |
| 13 | 96319_at | Cluster Incl AW061324 UI-M-BH1-anw-e-09-0-UI s1 Mus musculus cDNA, 3 end/clone = UI-M-BH1-anw-e-09-0-UI/ | 851.8 | P | 984.7 | P | 946.8 | P | 865.4 |
| | | clone_end = 3/gb = AW061324/gi = 6009075/ug = Mm 29931/len = 401/STRA = for | | | | | | | |
| 13 | 97516_at | Cluster Incl U92793 Mus musculus alpha glucosidase II alpha subunit mRNA, complete cds/cds = (11,2911)/gb = U92793/ | 294.1 | P | 237.7 | P | 400.5 | P | 305 |
| | | gi = 2104688/ug = Mm 3196/len = 3859/STRA = for | | | | | | | |
| 13 | 98076_at | Cluster Incl AI835644 UI-M-AJ0-aau-c-02-0-UI s1 Mus musculus cDNA, 3 end/clone = UI-M-AJ0-aau-c-02-0-UI/ | 723.2 | P | 853.1 | P | 562.4 | P | 543.2 |
| | | clone_end = 3/gb = AI835644/gi = 5469857/ug = Mm 34536/len = 372/STRA = for | | | | | | | |
| 13 | 98617_at | Cluster Incl AF071186 Mus musculus WW domain binding protein 11 mRNA, complete cds/cds = (7,1176)/gb = AF071186/ | 616.6 | P | 659.1 | P | 637.3 | P | 568 |
| | | gi = 3550081/ug = Mm 3855/len = 1864/STRA = for | | | | | | | |
| 13 | 99123_s_at | Cluster Incl AW061280 UI-M-BH1-anw-a-07-0-UI s1 Mus musculus cDNA, 3 end/clone = UI-M-BH1-anw-a-07-0-UI/ | 125.9 | P | 156.5 | P | 66.9 | P | 187.7 |
| | | clone_end = 3/gb = AW061280/gi = 6009031/ug = Mm 4050/len = 155/STRA = for | | | | | | | |
| 13 | 100156_at | Cluster Incl D26090 Mouse mRNA for mCDC46 protein, complete cds/cds = (84,2285)/gb = D26090/gi = 940403/ | 860.9 | P | 774.3 | P | 454 | P | 655.9 |
| | | ug = Mm 5048/len = 2649/STRA = for | | | | | | | |
| 13 | 100559_at | Cluster Incl AI853344.UI-M-BH0-air-b-10-0-UI.s1 Mus musculus cDNA, 3 end/clone = UI-M-BH0-air-b-10-0-UI/ | 280 | P | 259.4 | P | 269.1 | P | 262.7 |
| | | clone_end = 3/gb = AI853344/gi = 5497250/ug = Mm 5624/len = 336/STRA = for | | | | | | | |
| 13 | 100564_at | Cluster Incl AF068199 D-dopachrome tautomerase/cds = (0,356)/gb = AF068199/gi = 3452318/ug = Mm 5731/len = 357/ | 135.1 | P | 160.6 | P | 191 | P | 206.9 |
| | | STRA = for | | | | | | | |
| 13 | 102354_at | Cluster Incl AI049398 ub33c08 r1 Mus musculus cDNA, 5 end/clone = IMAGE-1379534/clone_end = 5/gb = AI049398/ | 408.1 | P | 361.2 | P | 231.1 | P | 204.2 |
| | | gi = 3297685/ug = Mm 11434/len = 420/STRA = for | | | | | | | |
| 13 | 102383_at | Cluster Incl AW048977.UI-M-BH1-amo-h-10-0-UI s1 Mus musculus cDNA, 3 end/clone = UI-M-BH1-amo-h-10-0-UI/ | 188.6 | P | 258.4 | P | 217.6 | P | 122.8 |
| | | clone_end = 3/gb = AW048977/gi = 5909506/ug = Mm 12180/len = 470/STRA = for | | | | | | | |
| 13 | 103799_at | Cluster Incl AF073881 Mus musculus myotubulann homologous protein 3 mRNA, partial cds/cds = (0,365)/gb = AF073881/ | 79.8 | P | 93.8 | P | 89 | P | 72.6 |
| | | gi = 3916225/ug = Mm 20844/len = 940/STRA = for | | | | | | | |
| 13 | 103889_at | Cluster Incl AI834833.UI-M-AM0-ads-g-05-0-UI s1 Mus musculus cDNA, 3 end/clone = UI-M-AM0-ads-g-05-0-UI/ | 408.7 | P | 368.2 | P | 285.7 | P | 274.4 |
| | | clone_end = 3/gb = AI834833/gi = 5469046/ug = Mm 21286/len = 407/STRA = for | | | | | | | |
| 13 | 95527_at | Cluster Incl AJ132771 Mus musculus mRNA for chromatin assembly factor-1 p150 subunit/cds = (0,2735)/gb = AJ132771/ | 44.9 | A | 157.1 | P | 146.6 | P | 91.5 |
| | | gi = 5852157/ug = Mm 27609/len = 2802/STRA = for | | | | | | | |
| 13 | 95541_at | Cluster Incl AW125506: UI-M-BH2.2-aqm-e-08-0-UI.s1 Mus musculus cDNA, 3 end/clone = UI-M-BH2.2-aqm-e-08-0-UI/ | 72.9 | P | 87.1 | P | 89.8 | P | 75 |
| | | clone_end = 3/gb = AW125506/gi = 6101036/ug = Mm 27673/len = 434/STRA = rev | | | | | | | |
| 13 | 95634_at | Cluster Incl AI848107 UI-M-AP1-agk-e-09-0-UI s1 Mus musculus cDNA, 3 end/clone = UI-M-AP1-agk-e-09-0-UI/ | 178 | P | 189.6 | P | 143.7 | P | 134.6 |
| | | clone_end = 3/gb = AI848107/gi = 5492013/ug = Mm 28094/len = 475/STRA = for | | | | | | | |
| 13 | 96753_at | Cluster Incl Y11905 M. musculus BCL7C mRNA/cds = (57,710)/gb = Y11905/gi = 1946223/ug = Mm 2898/len = 781/STRA = for | 238.9 | P | 188.5 | P | 321 | P | 265.8 |
| 13 | 96763_at | Cluster Incl AI839995: UI-M-AH0-acs-f-11-0-UI s1 Mus musculus cDNA, 3 end/clone = UI-M-AH0-acs-f-11-0-UI/ | 150.1 | P | 135.8 | P | 115.8 | P | 125.3 |
| | | clone_end = 3/gb = AI839995/gi = 5474208/ug = Mm 29007/len = 357/STRA = for | | | | | | | |
| 13 | 98437_at | Cluster Incl U63720 Mus musculus CPP32 apoptotic protease mRNA, partial cds/cds = (0,662)/gb = U63720/gi = 4097524/ | 771.4 | P | 896.5 | P | 712.4 | P | 680.4 |
| | | ug = Mm 34405/len = 1609/STRA = for | | | | | | | |
| 13 | 100513_at | Cluster Incl AF075461 Mus musculus ADP-nbosylation factor-directed GTPase activating protein isoform a (Shag1) mRNA, | −34.9 | P | 11.2 | P | −200.7 | P | 117.1 |
| | | complete cds/cds = (49,3492)/gb = AF075461/gi = 4063613/ug = Mm 5237/len = 4370/STRA = rev | | | | | | | |
| 13 | 100989_at | Cluster Incl AJ001373 Mus musculus bodenin gene/cds = (173,775)/gb = AJ001373/gi = 2647955/ug = Mm 6818/len = 954/ | 152.9 | P | 178.6 | P | 162.4 | P | 189.1 |
| | | STRA = for | | | | | | | |
| 13 | 101854_r_at | Cluster Incl AI844178 UI-M-AL1-ahk-a-02-0-UI s1 Mus musculus cDNA, 3 end/clone = UI-M-AL1-ahl-a-02-0-UI/ | 1024.8 | P | 1050.5 | P | 1061.2 | P | 850.7 |
| | | clone_end = 3/gb = AI844178/gi = 5488084/ug = Mm 866/len = 298/STRA = for | | | | | | | |
| 13 | 103711_at | Cluster Incl AI115399: uh85b06.r1 Mus musculus cDNA, 5 end/clone = IMAGE-1764467/clone_end = 5/gb = AI115399/ | 156.5 | P | 151.9 | P | 123.1 | P | 78.3 |
| | | gi = 3515723/ug = Mm 24100/len = 450/STRA = for | | | | | | | |
| 13 | 92758_at | Cluster Incl U09268 Mus musculus domesticus 129 tyrosine-threonine dual specificity phosphatase PAC-1 gene, complete | 724.8 | P | 559.1 | P | 734.8 | P | 664.1 |
| | | cds/cds = (0,539)/gb = U09268/gi = 505624/ug = Mm 4729/len = 540/STRA = for | | | | | | | |
| 13 | 93698_at | Cluster Incl AB006329 Mus musculus mSox13 mRNA, complete cds/cds = (48,1835)/gb = AB006329/gi = 3077735/ | 1303.7 | P | 952.4 | P | 1219.4 | P | 1216.2 |
| | | ug = Mm 8575/len = 3183/STRA = rev | | | | | | | |
| 13 | 95374_f_at | Cluster Incl AI413993 ma76g11.x1 Mus musculus cDNA, 3 end/clone = IMAGE-316676/clone_end = 3/gb = AI413993/ | 624.8 | P | 690.8 | P | 872.3 | P | 754.4 |
| | | gi = 4257497/ug = Mm 22881/len = 308/STRA = for | | | | | | | |
| 13 | AFFX- | L09192 Mus musculus pyruvate carboxylase mRNA, complete cds (_5, _MA, MB, _3 | 465.7 | P | 522.8 | P | 562.1 | P | 402.4 |
| | PyruCarbMur/ | represent transcript regions 5 prime, | | | | | | | |
| | L09192_3_at | | | | | | | | |
| | | MiddleA, MiddleB, and 3 prime respectively) | | | | | | | |
| 13 | 92879_at | Cluster Incl U42383 Fibroblast growth factor inducible 13/cds = (9,1637)/gb = U42383/gi = 3318654/ug = Mm 14501/ | 574.7 | P | 468.6 | P | 320.7 | P | 501 |
| | | len = 2069/STRA = for | | | | | | | |
| 13 | 93803_at | Cluster Incl AB007139 Mus musculus Psme3 gene for PA28 gamma subunit, complete cds/cds = (119,883)/gb = AB007139/ | 314.4 | P | 286.5 | P | 301.9 | P | 272 |
| | | gi = 3219287/ug = Mm 2141/len = 2555/STRA = for | | | | | | | |
| 13 | 94015_at | Cluster Incl AW060447 UI-M-BH1-anj-f-02-0-UI s1 Mus musculus cDNA, 3 end/clone = UI-M-BH1-anj-f-02-0-UI/ | 295.2 | P | 239.1 | P | 243.5 | P | 345.1 |
| | | clone_end = 3/gb = AW060447/gi = 6006198/ug = Mm 2184/len = 460/STRA = for | | | | | | | |
| 13 | 97271_at | Cluster Incl AI845593 UI-M-AQ1-adx-d-04-0-UI.s1 Mus musculus cDNA, 3 end/clone = UI-M-AQ1-adx-d-04-0-UI/ | 373.3 | P | 359 | P | 241.1 | P | 266.7 |
| | | clone_end = 3/gb = AI845593/gi = 5489499/ug = Mm.27922/len = 390/STRA = for | | | | | | | |
| 13 | 98069_s_at | Cluster Incl AI854467 UI-M-BH0-ait-g-02-0-UI s1 Mus musculus cDNA, 3 end/clone = UI-M-BH0-ait-g-02-0-UI/clone_end = 3/ | 238.1 | P | 211.8 | P | 287 | P | 223.3 |
| | | gb = AI854467/gi = 5498373/ug = Mm 34456/len = 390/STRA = for | | | | | | | |
| 13 | 101485_at | Cluster Incl AW124859 UI-M-BH2.1-apu-a-02-0-UI.s1 Mus musculus cDNA, 3 end/clone = UI-M-BH2.1-apu-a-02-0-UI/ | 393.1 | P | 377.9 | P | 365.9 | P | 339.6 |
| | | clone_end = 3/gb = AW124859/gi = 6100389/ug = Mm 7861/len = 372/STRA = for | | | | | | | |
| 13 | 101967_at | Cluster Incl D50646: Mouse mRNA for SDF2, complete cds/cds = (28,663)/gb = D50646/gi = 1741869/ug = Mm.935/len = 1012/ | 541.6 | P | 576.5 | P | 475.1 | P | 468.5 |
| | | STRA = for | | | | | | | |
| 13 | 103884_at | Cluster Incl AI850335: UI-M-BG1-alf-g-11-0-UI.s1 Mus musculus cDNA, 3 end/clone = UI-M-BG1-aif-g-11-0-UI/ | −125.3 | P | −221.9 | P | −131.4 | P | −244.5 |
| | | clone_end = 3/gb = AI850335/gi = 5494241/ug = Mm 21241/len = 475/STRA = for | | | | | | | |
| 13 | 104583_at | Cluster Incl AI227492 uf10c07 y1 Mus musculus cDNA, 5 end/clone = IMAGE-1510956/clone_end = 5/gb = AI227492/ | 130.7 | P | 241 | P | 187.8 | P | 204.8 |
| | | gi = 3810545/ug = Mm 23975/len = 655/STRA = for | | | | | | | |
| 13 | 95612_at | Cluster Incl AA667128 vq89a03.r1 Mus musculus cDNA, 5 end/clone = IMAGE-1109452/clone_end = 5/gb = AA667128/ | 407.4 | P | 626.8 | P | 322.4 | P | 331.7 |
| | | gi = 2625829/ug = Mm.27997/len = 108/STRA = for | | | | | | | |
| 13 | 96125_at | Cluster Incl AF110520: Mus musculus major histocompatibllity complex region NG27, NG28, RPS28, NADH | 515.2 | P | 530.1 | P | 484.9 | P | 480.3 |
| | | oxidoreductase, NG29, KIFC1, Fas-binding protein, BING1, tapasin, RalGDS-like, KE2, BING4, beta 1,3-galactosyl | | | | | | | |
| | | transferase, and RPS18 genes, complete cds, S/STRA = for | | | | | | | |
| 13 | 96234_at | Clusler Incl AI849311, UI-M-AJ1-aha-c-04-0-UI.s1 Mus musculus cDNA, 3 end/clone = UI-M-AJ1-aha-c-04-0-UI/ | 224.7 | P | 195.2 | P | 152.4 | P | 201.1 |
| | | clone_end = 3/gb = AI849311/gi = 5493217/ug = Mm 28790/len = 471/STRA = for | | | | | | | |
| 13 | 100004_at | Cluster Incl AW120890: UI-M-BH2.3-aob-a-12-0-UI s1 Mus musculus cDNA, 3 end/clone = UI-M-BH2 3-aob-a-12-0-UI/ | 398.4 | P | 402.7 | P | 319.4 | P | 436.1 |
| | | clone_end = 3/gb = AW120890/gi = 6096223/ug = Mm 45194/len = 364/STRA = for | | | | | | | |
| 13 | 104259_at | Cluster Incl X99641 M musculus mRNA for HP1 alpha protein/cds = (63,638)/gb = X99641/gi = 1480107/ug = Mm 28408/ | 366.2 | P | 243.6 | P | 319.8 | P | 283.7 |
| | | len = 865/STRA = for | | | | | | | |
| 13 | 92332_at | Cluster Incl M80540 Distal-less homeobox 2/cds = (300,1298)/gb = M80540/gi = 201920/ug = Mm 3896/len = 2408/STRA = for | 113.2 | P | 113.4 | P | 28.4 | P | 34.3 |
| 13 | 98355_at | Cluster Incl AA544993-vk42e09 r1 Mus musculus cDNA, 5 end/clone = IMAGE-949288/clone_end = 5/gb = AA544993/ | 402 | P | 263.1 | P | 379.3 | P | 408.1 |
| | | gi = 2306067/ug = Mm 4393/len = 297/STRA = for | | | | | | | |
| 13 | 99807_r_at | Cluster Incl X97720 Friend virus susceptibility 1/cds = (0,1322)/gb = X97720/gi = 1515301/ug = Mm 57220/len = 1323 | 246.2 | P | 119.5 | A | 320.9 | P | 242.6 |
| | | STRA = for | | | | | | | |
| 13 | 100684_at | Cluster Incl U92794-Proteln kinase C substrate 80K-H/cds = (114,1679)/gb = U92794/gi = 2104690/ug = Mm 14244/len = 2030/ | 392.9 | P | 524.5 | P | 471.8 | P | 445.9 |
| | | STRA = for | | | | | | | |
| 13 | 101508_at | Cluster Incl U17961: Mus musculus p62 mRNA, complete cds/cds = (111,1442)/gb = U17961/gi = 602250/ug = Mm 8256/ | 238 | P | 242.9 | P | 255.2 | P | 164 |
| | | len = 1765/STRA = for | | | | | | | |
| 13 | 103625_at | Cluster Incl AA797556 vw25a08 r1 Mus musculus cDNA, 5 end/clone = IMAGE-1244822/clone_end = 5/gb = AA797556/ | 360.3 | P | 348.4 | P | 281.9 | P | 301.9 |
| | | gi = 2860511/ug = Mm 20279/len = 427/STRA = for | | | | | | | |
| 13 | 104301_at | Cluster Incl AI852414 UI-M-BH0-aip-e-01-0-UI s1 Mus musculus cDNA, 3 end/clone = UI-M-BH0-aip-e-01-0-UI/ | 327.7 | P | 310.7 | P | 232.8 | P | 325.8 |
| | | clone_end = 3/gb = AI852414/gi = 5496360/ug = Mm.22351/len = 425/STRA = for | | | | | | | |
| 13 | 94356_at | Cluster Incl AI593074 vv10f08.x1 Mus musculus cDNA, 3 end/clone = IMAGE-1211267/clone_end = 3/gb = AI593074/ | 93.5 | P | 99.3 | P | 50.7 | P | 66.5 |
| | | gi = 4602122/ug = Mm 25231/len = 604/STRA = rev | | | | | | | |
| 13 | 96189_at | Cluster Incl AW046184 UI-M-BH1-akw-d-10-0-UI s1 Mus musculus cDNA, 3 end/clone = UI-M-BH1-akw-d-10-0-UI/ | 44.1 | P | 49.3 | P | 20.5 | P | 28.1 |
| | | clone_end = 3/gb = AW046184/gi = 5906713/ug = Mm 28502/len = 416/STRA = for | | | | | | | |
| 13 | 99023_at | Cluster Incl U57747 Mus musculus platelet-activating factor acetylhydrolase isoform Ib beta subunit mRNA, complete cds/ | 446.5 | A | 313.5 | P | 389.2 | P | 347.8 |
| | | cds = (106,795)/gb = U57747/gi = 1373362/ug = Mm 3877/len = 944/STRA = for | | | | | | | |
| 13 | 100461_at | Cluster Incl D85818 Mouse mRNA for RNA polymerase II subuunit RPB14, complete cds/cds = (16,369)/gb = D85818/ | 507.2 | P | 558.9 | P | 433.4 | A | 573.7 |
| | | gi = 2081615/ug = Mm 4896/len = 531/STRA = for | | | | | | | |
| 13 | 100908_at | Cluster Incl M36033 Protein tyrosine phosphatase, receptor type, A/cds = (25,2514)/gb = M36033/gi = 198876/ug = Mm 587/ | 177.8 | P | 132.2 | P | 94.1 | P | 105.8 |
| | | len = 3070/STRA = for | | | | | | | |
| 13 | 101856_at | Cluster Incl AI836771.UI-M-AJ0-abc-d-02-0-UI.s1 Mus musculus cDNA, 3 end/clone = UI-M-AJ0-abc-d-02-0-UI/ | 218.2 | P | 287.2 | P | 247.1 | P | 185.1 |
| | | clone_end = 3/gb = AI836771/gi = 5470964/ug = Mm 86933/len = 200/STRA = for | | | | | | | |
| 13 | 95765_at | Cluster Incl U48971 Mus musculus class III alcohol dehydrogenase pseudogene/cds = (0,1124)/gb = U48971/gi = 1399469/ | 462.5 | P | 522.1 | P | 447.6 | P | 347 |
| | | ug = Mm 87751/len = 1125/STRA = for | | | | | | | |
| 13 | 97249_at | Cluster Incl AW124204 UI-M-BH2.1-aph-b-11-0-UI s1 Mus musculus cDNA, 3 end/clone = UI-M-BH2 1-aph-b-11-0-UI/ | 335.5 | P | 343.6 | A | 340.1 | P | 283.1 |
| | | clone_end = 3/gb = AW124204/gi = 6099734/ug = Mm 27853/len = 382/STRA = for | | | | | | | |
| 13 | 95759_at | Cluster Incl AW123746: UI-M-BH2 1-apl-h-09-0-UI s1 Mus musculus cDNA, 3 end/clone = UI-M-BH2 1-apl-h-09-0-UI/ | 98.3 | P | 83.9 | P | 11.4 | A | 81.9 |
| | | clone_end = 3/gb = AW123746/gi = 6099276/ug = Mm 298/len = 462/STRA-for | | | | | | | |
| 13 | 99139_at | Cluster Incl AW123783 UI-M-BH2 1-apm-d-01-0-UI s1 Mus musculus cDNA, 3 end/clone = UI-M-BH2.1-apm-d-01-0-UI/ | 411.3 | P | 433.3 | P | 443.5 | P | 367.5 |
| | | clone_end = 3/gb = AW123783/gi = 6099313/ug = Mm 41374/len = 404/STRA = for | | | | | | | |
| 13 | 101054_at | Cluster Incl X00496 Mouse la-associated invariant chain (II) mRNA fragment/cds = (0,509)/gb = X00496/gi = 53102/ | 462.3 | P | 257 | P | 395.2 | P | 220.9 |
| | | ug = Mm.7043/len = 994/STRA = for | | | | | | | |
| 13 | 101074_at | Cluster Incl D89063: Mus musculus mRNA for oligosaccharyltransferase, complete cds/cds = (143,1468)/gb = D89063/ | 172.6 | P | 224.8 | P | 225.4 | P | 164.4 |
| | | gi = 2687875/ug = Mm 7236/len = 1750/STRA = for | | | | | | | |
| 13 | 103871_at | Cluster Incl AW123729 UI-M-BH2 1-apl-f-11-0-UI s1 Mus musculus cDNA, 3 end/clone = UI-M-BH2 1-apl-f-11-0-UI/ | 95.3 | P | 74.4 | A | 35.2 | P | 159.7 |
| | | clone_end = 3/gb = AW123729/gi = 6099259/ug = Mm 21163/len = 462/STRA = for | | | | | | | |
| 13 | 94407_at | Cluster Incl AI835481 UI-M-AQ0-aag-e-02-0-UI s1 Mus muaculus cDNA, 3 end/clone = UI-M-AQ0-aag-e-02-0-UI/ | 251.8 | P | 269.3 | P | 290.9 | P | 228.4 |
| | | clone_end = 3/gb = AI835481/gi = 5469694/ug = Mm 259/len = 220/STRA = for | | | | | | | |
| 13 | 96121_at | Cluster Incl AW047866 UI-M-BH1-als-f-05-0-UI s1 Mus musculus cDNA, 3 end/clone = UI-M-BH1-als-f-05-0-UI/ | 288.2 | P | 309 | P | 252.5 | P | 279.6 |
| | | clone_end = 3/gb = AW047866/gi = 5908395/ug = Mm 28103/len = 411/STRA = rev | | | | | | | |
| 13 | 94644_at | Cluster Incl M99492 Mouse butyrylcholinesterase mRNA, complete cds/cds = (17,1828)/gb = M99492/gi = 191579/ | 111.7 | A | 59.2 | P | 36.3 | P | −11.9 |
| | | ug = Mm.8073/len = 1891/STRA = for | | | | | | | |
| 13 | 93303_at | Cluster Incl U64445 Ubiquitin fusion degradation 1 —like/cds = (106,1029)/gb = u64445/gi = 1654347/ug = Mm.1830/ | 122.8 | P | 164.4 | A | 139.7 | P | 117.6 |
| | | len = 1479/STRA = for | | | | | | | |
| 13 | 97238_at | Cluster Incl AW209238: uo07g04.x1 Mus musculus cDNA, 3 end/clone = IMAGE-2598870/clone_end = 3/gb = AW209238/ | 64.4 | P | 66.4 | P | 52.2 | P | 40.2 |
| | | gi = 6515178/ug = Mm 27836/len = 568/STRA = for | | | | | | | |
| 13 | 97251_at | Cluster Incl AI842124: UI-M-AN1-afg-h-03-0-UI s1 Mus musculus cDNA, 3 end/clone = UI-M-AN1-afg-h-03-0-UI/ | 148.2 | P | 154.9 | P | 150.8 | P | 119.1 |
| | | clone_end = 3/gb = AI842124/gi = 5476337/ug = Mm 27863/len = 301/STRA = for | | | | | | | |
| 13 | 100526_f_at | Cluster Incl X84227 Cyritestin 1/cds = (72,2540)/gb = X64227/gi = 1666660/ug = Mm 5168/len = 2722/STRA = for | 144.7 | A | 153.2 | A | 60.5 | P | 134.9 |
| 13 | 94382_at | Cluster Incl AI851901.UI-M-BH0-aix-d-03-0-UI s1 Mus musculus cDNA, 3 end/clone = UI-M-BH0-aix-d-03-0-UI/ | 119.6 | P | 87.2 | P | 109.3 | A | 105.8 |
| | | clone_end = 3/gb = AI851901/gi = 5495807/ug = Mm 25608/len = 428/STRA = for | | | | | | | |
| 13 | 99459_f_at | Cluster Incl X70764: ELKL motif kinase/cds = (93,2417)/gb = X70764/gi = 57919/ug = Mm.4082/len = 2763/STRA = for | 70.3 | M | 92.9 | P | 107.7 | P | 101.6 |
| 13 | 100026_at | Cluster Incl U42443: Mus musculus MECA39 mRNA, complete cds/cds = (0,1160)/gb = U42443/gi = 1480454/ug = Mm.4606/ | 168.2 | P | 174.6 | A | 231.3 | P | 228.3 |
| | | len = 1161/STRA = for | | | | | | | |
| 13 | 93612_at | Cluster Incl D86332 Matrix metalloproteinase 15/cds = (28,2001)/gb = D86332/gi = 2687872/ug = Mm 7283/len = 3339/ | 31.7 | P | 102.6 | A | 157.5 | P | 99.9 |
| | | STRA = for | | | | | | | |
| 13 | 94713_at | Cluster Incl U81453 Myosin VIIa/cds = (10,6657)/gb = U81453/gi = 1778381/ug = Mm 1403/len = 7135/STRA = rev | 294.7 | P | 326.3 | P | 302.5 | A | 308.3 |
| 13 | 95514_at | Cluster Incl AI846302 UI-M-AK1-aez-g-04-0-UI s1 Mus musculus cDNA, 3 end/clone = UI-M-AK1-aez-g-04-0-UI/ | 172.8 | P | 92.7 | M | 240.5 | A | 148.7 |
| | | clone_end = 3/gb = AI846302/gi = 5490208/ug = Mm 253/len = 324/STRA = rev | | | | | | | |
| 13 | 104078_g_at | Cluster Incl AW121992 UI-M-BH2 3-aoj-f-11-0-UI.s1 Mus musculus cDNA, 3 end/clone = UI-M-BH2.3-aoj-f-11-0-UI/ | 118.2 | A | 49.1 | P | 52.5 | M | 71.2 |
| | | clone_end = 3/gb = AW121992/gi = 6097398/ug = Mm 21705/len = 477/STRA = for | | | | | | | |
| 13 | 94933_at | Cluster Incl AI853739 UI-M-BH0-ajg-a-12-0-UI.s1 Mus musculus cDNA, 3 end/clone = UI-M-BH0-ajg-a-12-0-UI/ | 339.7 | A | 351.1 | A | 332.2 | A | 345 |
| | | clone_end = 3/gb = AI853739/gi = 5497645/ug = Mm.26783/len = 346/STRA = for | | | | | | | |
| 13 | 98493_at | Cluster Incl AA867026 vx22e02 r1 Mus musculus cDNA, 5 end/clone = IMAGE-1265210/clone_end = 5/gb = AA867026/ | 61.1 | P | 9.5 | A | −4.1 | M | 87.5 |
| | | gi = 2962471/ug = Mm 35606/len = 269/STRA = for | | | | | | | |
| 13 | 98994_at | Cluster Incl AF081499 Mus musculus type IIb Na/phosphate-cotransporter (Npt2b) mRNA, complete cds/cds = (44,2137)/ | 95.6 | M | 101.6 | P | 117.6 | P | 26.9 |
| | | gb = AF081499/gi = 3929576/ug = Mm 3786/len = 4039/STRA = for | | | | | | | |
| 13 | 99548_at | Cluster Incl AF033034 Mus musculus aldehyde dehydrogenase 3 (aldh3) gene, partial cds/cds = (45,155)/gb = AF033034/ | −469 | P | −219.9 | P | −242.8 | P | −246.5 |
| | | gi = 2645710/ug = Mm 4257/len = 156/STRA = for | | | | | | | |
| 13 | 99961_s_at | Cluster Incl L37092 Mus musculus cyclin-dependent kinase homologue (p130PITSL) mRNA, complete cds/cds = (661,3012)/ | 17.5 | P | 14 | P | 54.1 | A | 19.7 |
| | | gb = L37092/gi = 561745/ug = Mm 4414/len = 3161/STRA = for | | | | | | | |
| 13 | 96976_f_at | Cluster Incl X16217 Mouse T11 class I MHC gene (exon 5)/cds = (0,88)/gb = X16217/gi = 54221/ug = Mm 88813/len = 113/ | 308.4 | P | 183 | M | 167 | M | 258.9 |
| | | STRA = for | | | | | | | |
| 14 | 92571_at | Cluster Incl D85904 Mouse mRNA for apg-2, complete cds/cds = (15,2540)/gb = D85904/gi = 1389636/ug = Mm 1032/ | 327.9 | P | 457.1 | P | 335.6 | P | 355 |
| | | len = 2582/STRA = for | | | | | | | |
| 14 | 92580_at | Cluster Incl U39473 Mus musculus histidyl-tRNA synthetase mRNA, complete cds/cds = (85,1614)/gb = U39473/ | 228.5 | P | 330 | P | 297.8 | P | 348.8 |
| | | gi = 1209682/ug = Mm 10528/len = 1629/STRA = for | | | | | | | |
| 14 | 92598_at | Cluster Incl AI843029.UI-M-AK1-aeq-e-08-0-UI s1 Mus musculus cDNA, 3 end/clone = UI-M-AK1-aeq-e-08-0-UI/ | 992.5 | P | 1019.9 | P | 885.9 | P | 1059.5 |
| | | clone_end = 3/gb = AI843029/gi = 5477242/ug = Mm 10727/len = 298/STRA = for | | | | | | | |
| 14 | 93059_at | Cluster Incl AI854841 UI-M-BH0-akb-b-10-0-UI s1 Mus musculus cDNA, 3 end/clone = UI-M-BH0-akd-b-10-0-UI/ | 320.5 | P | 412.1 | P | 302.9 | P | 303.2 |
| | | clone_end = 3/gb = AI854841/gi = 5498747/ug = Mm 155/len = 396/STRA = for | | | | | | | |
| 14 | 93256_at | Cluster Incl AI853728 UI-M-BH0-ajq-e-09-0-UI s1 Mus musculus cDNA, 3 end/clone = UI-M.BH0-ajq-e-09-0-UI/ | 933.5 | P | 937.9 | P | 856.5 | P | 955.5 |
| | | clone_end = 3/gb = AI853728/gi = 5497599/ug = Mm 17035/len = 482/STRA = for | | | | | | | |
| 14 | 93312_at | Cluster Incl AW124623 UI-M-BH2 1-apw-b-04-0-UI s1 Mus musculus cDNA, 3 end/clone = UI-M-BH2.1-apw-b-4-0-UI/ | 617.1 | P | 703.3 | P | 632.5 | P | 669.1 |
| | | clone_end = 3/gb = AW124623/gi = 6100153/ug = Mm 18485/len = 422/STRA = for | | | | | | | |
| 14 | 93319_at | Cluster Incl U20238 Mus musculus GTPase-activating protein GAPIII mRNA, complete cds/cds = (125,2629)/gb-U20238/ | 435.1 | P | 489.5 | P | 401.6 | P | 356 |
| | | gi = 972943/ug = Mm.18517/len = 4107/STRA = for | | | | | | | |
| 14 | 93580_at | Cluster Incl AI837010 UI-M-AJ0-aaz-h-06-0-UI s1 Mus musculus cDNA, 3 end/clone = UI-M-AJ0-aaz-h-06-0-UI/ | 940.1 | P | 884.9 | P | 706.3 | P | 805.8 |
| | | clone_end = 3/gb = AI837010/gi = 5471223/ug = Mm 20543/len = 472/STRA = for | | | | | | | |
| 14 | 94018_at | Cluster Incl AW120725 UI-M-BH2 3-any-a-12-0-UI s1 Mus musculus cDNA, 3 end/clone = UI-M-BH2 3-any-a-12-0-UI/ | 1306.7 | P | 1228.3 | P | 1554.1 | P | 1530 |
| | | clone_end = 3/gb = AW120725/gi = 6096058/ug = Mm 21846/len = 227/STRA = for | | | | | | | |
| 14 | 94245_at | Cluster Incl AW125865 UI-M-BH2 2-aqk-e-12-0-UI s1 Mus musculus cDNA, 3 end/clone = UI-M-BH2 2-aqk-e-12-0-UI/ | 196.5 | P | 235.3 | P | 123.4 | P | 213.5 |
| | | clone_end = 3/gb = AW125865/gi = 6101395/ug = Mm 22362/len = 178/STRA = for | | | | | | | |
| 14 | 94834_at | Cluster Incl U06119 Cathepsin H/cds = (47,1048)/gb = U06119/gi = 454100/ug = Mm 2277/len = 1381/STRA = for | 533 | P | 549.2 | P | 690.7 | P | 610.7 |
| 14 | 94893_at | Cluster Incl AI840411 UI-M-AH0-acv-h-03-0-UI.s1 Mus musculus cDNA, 3 end/clone = UI-M-AH0-acv-h-03-0-UI/ | 131.4 | P | 165.5 | P | 100 | P | 59.4 |
| | | clone_end = 3/gb = AI840411/gi = 5474624/ug = Mm.23962/len = 435/STRA = for | | | | | | | |
| 14 | 95425_at | Cluster Incl U21489 Acetyl coenzyme A dehydrogenase, long-chain/cds = (0,1292)/gb = U21489/gi = 726094/ug = Mm.2445/ | 307.4 | P | 343.3 | P | 301.7 | P | 428.3 |
| | | len = 1434/STRA = for | | | | | | | |
| 14 | 96038_at | Cluster Incl AI840339 UI-M-AJ0-abd-g-01-0-UI S2 Mus musculus cDNA, 3 end/clone = UI-M-AJ0-abd-g-01-0-UI/ | 512.4 | P | 613.5 | P | 715.4 | P | 550.2 |
| | | clone_end = 3/gb = AI840339/gi = 5474552/ug = Mm 2587/len = 497/STRA = for | | | | | | | |
| 14 | 96079_at | Cluster Incl AI853881 UI-M-BH0-aju-a-01-0-UI s1 Mus musculus cDNA, 3 end/clone = UI-M-BH0-aju-a-01-0-UI/ | 339.6 | P | 356.2 | P | 289.2 | P | 316.1 |
| | | clone_end = 3/gb = AI853881/gi = 5497787/ug = Mm 26598/len = 313/STRA = for | | | | | | | |
| 14 | 96088_at | Cluster Incl AB033921 Mus musculus mRNA for Ndr1 related protein Ndr2, complete cds/cds = (35,1150)/gb = AB033921/ | 407.4 | P | 348 | P | 436.4 | P | 300.5 |
| | | gi = 6141565/ug = Mm 26722/len = 2010/STRA = for | | | | | | | |
| 14 | 96680_at | Cluster Incl AI835630 UI-M-AI0-aap-h-02-0-UI a1 Mus musculus cDNA, 3 end/clone = UI-M-AI0-aap-h-02-0-UI/ | 186.4 | P | 168 | P | 165.3 | P | 215.3 |
| | | clone_end = 3/gb = AI835630/gi = 5469843/ug = Mm 27432/len = 327/STRA = for | | | | | | | |
| 14 | 97217_at | Cluster Incl AI842095 UI-M-AN1-afg-e-02-0-UI.s1 Mus musculus cDNA, 3 end/clone = UI-M-AN1-afg-e-02-0-UI/ | 1315.8 | P | 1420 | P | 1385.4 | P | 1332.6 |
| | | clone_end = 3/gb = AI842095/gi = 5476308/ug = Mm 27733/len = 418/STRA = for | | | | | | | |
| 14 | 97241_at | Cluster Incl AI787713 ul21fo4.y1 Mus musculus cDNA, 5 end/clone = IMAGE-2088223/clone_end = 5/gb = AI787713/ | 391 | P | 496.2 | P | 432.6 | P | 417.4 |
| | | gi = 5335420/ug = Mm 27839/len = 528/STRA = for | | | | | | | |
| 14 | 97802_at | Cluster Incl AI850195: UI-M-BG1-aie-a-03-0-UI.s1 Mus musculus cDNA, 3 end/clone = UI-M-BG1-aie-a-03-0-UI/ | 295.2 | P | 221.4 | P | 225.4 | P | 238.6 |
| | | clone_end = 3/gb = AI850195/gi = 5494101/ug = Mm28128/len = 531/STRA = for | | | | | | | |
| 14 | 97868_at | Cluster Incl AW060270.UI-M-BH1-amv-g-11-0-UI s1 Mus musculus cDNA, 3 end/clone = UI-M-BH1-amv-g-11-0-UI/ | 439.8 | P | 724.2 | P | 546.6 | P | 711.8 |
| | | clone_end = 3/gb = AW060270/gi = 6008061/ug = Mm 28330/len = 515/STRA = for | | | | | | | |
| 14 | 98880_at | Cluster Incl AW124955-UI-M-BH2 1-apv-a-10-0-UI s1 Mus musculus cDNA, 3 end/clone = UI-M-BH2 1-apv-a-10-0-UI/ | 522.4 | P | 499.5 | P | 619.6 | P | 535 |
| | | clone_end = 3/gb = AW124955/gi = 6100485/ug = Mm28524/len = 316/STRA = for | | | | | | | |
| 14 | 98931_at | Cluster Incl AW123589: UI-M-BH2 1-api-b-04-0-UI s1 Mus musculus cDNA, 3 end/clone = UI-M-BH2 1-api-b-04-0-UI/ | 276.1 | P | 268.6 | P | 377.2 | P | 275.6 |
| | | clone_end = 3/gb = AW123589/gi = 6099119/ug = Mm 26670/len = 421/STRA = for | | | | | | | |
| 14 | 98945_at | Cluster Incl AW125190.UI-M-BH2 1-apz-h-07-0-UI s1 Mus musculus cDNA, 3 end/clone = UI-M-BH2 1-apz-h-07-0-UI/ | 293.2 | P | 349.4 | P | 375.6 | P | 421.4 |
| | | clone_end = 3/gb = AW125190/gi = 6100720/ug = Mm 28714/len = 396/STRA = for | | | | | | | |
| 14 | 98950_at | Cluster Incl AB017616 Mus musculus YGR163W mRNA homologue, complete cds/cds = (24,1220)/gb = AB017616/ | 844.1 | P | 810.6 | P | 837 | P | 681.5 |
| | | gi = 4519622/ug = Mm 28746/len = 2507/STRA = for | | | | | | | |
| 14 | 94469_at | Cluster Incl AW120950: UI-M-BH2.3-aod-b-02-0-UI.s1 Mus musculus cDNA, 3 end/clone = UI-M-BH2.3-aod-b-02-0-UI/ | 646.9 | P | 706.2 | P | 726.8 | P | 659.4 |
| | | clone_end = 3/gb = AW120950/gi = 6096283/ug = Mm.28848/len = 336/STRA = for | | | | | | | |
| 14 | 94470_i_at | Cluster Incl AW045974: UI-M-BH1-alc-a-08-0-UI s1 Mus musculus cDNA, 3 end/clone = UI-M-BH1-alc-a-08-0-UI/ | 266.9 | P | 225.3 | P | 188.8 | P | 263.7 |
| | | clone_end = 3/gb = AW045974/gi = 5906503/ug = Mm 28851/len = 338/STRA = for | | | | | | | |
| 14 | 94478_at | Cluster Incl AI841377 UI-M-AM0-adv-g-04-0-UI s1 Mus musculus cDNA, 3 end/clone = UI-M-AM0-adv-g-04-0-UI/ | 651 | P | 719 | P | 741.8 | P | 754.6 |
| | | clone_end = 3/gb = AI841377/gi = 5475590/ug = Mm.28872/len = 527/STRA = for | | | | | | | |
| 14 | 95103_at | Cluster Incl AI839824 UI-M-AN0-acq-c-09-0-UI s1 Mus musculus cDNA, 3 end/clone = UI-M-AN0-acq-c-09-0-UI/ | 597.4 | P | 608.1 | P | 477. 7 | P | 535.3 |
| | | clone_end = 3/gb = AI839824/gi = 5474002/ug = Mm 29348/len = 481/STRA = for | | | | | | | |
| 14 | 95695_at | Cluster Incl AB017112 Mus musculus mCAC gene, complete cds/cds = (0,905)/gb = AB017112/gi = 4239973/ug = Mm.29666/ | 414.5 | P | 421.8 | P | 425. 2 | P | 455.4 |
| | | len = 906/STRA = for | | | | | | | |
| 14 | 95722_at | Cluster Incl AB013137 Mus musculus mRNA for glutaredoxin, complete cds/cds = (49,372)/gb = AB013137/gi = 6451922/ | 1222.2 | P | 1476 | P | 1439 | P | 1841.6 |
| | | ug = Mm 29728/len = 1287/STRA = for | | | | | | | |
| 14 | 96840_at | Cluster Incl AI851243 UI-M-BH0-akf-b-01-0-UI s1 Mus musculus cDNA, 3 end/clone = UI-M-BH0-akf-b-01-0-UI/ | 847.8 | P | 871.1 | P | 751 | P | 827 |
| | | clone_end = 3/gb = AI851243/gi = 5495149/ug = Mm.30017/len = 430/STRA = for | | | | | | | |
| 14 | 96870_at | Cluster Incl AI836740 UI-M-AJ0-aax-h-06-0-UI s1 Mus musculus cDNA, 3 end/clone = UI-M-AJ0-aax-h-06-0-UI/ | 2110.7 | P | 2902.5 | P | 2508.4 | P | 2945.6 |
| | | clone_end = 3/gb = AI836740/gi = 5470958/ug = Mm.30065/len = 420/STRA = for | | | | | | | |
| 14 | 96900_at | Cluster Incl AW125480 UI-M-BH2 2-aqm-c-05-0-UI s1 Mus musculus cDNA, 3 end/clone = UI-M-BH2 2-aqm-c-05-0-UI/ | 271.8 | P | 793.1 | P | 618.8 | P | 777.4 |
| | | clone_end = 3/gb = AW125480/gi = 6101010/ug = Mm 30114/len = 476/STRA = for | | | | | | | |
| 14 | 97485_at | Cluster Incl AI850953 UI-M-BH0-akd-e-11-0-UI s1 Mus musculus cDNA, 3 end/clone = UI-M-BH0-akd-e-11-0-UI/ | 773.1 | P | 896.9 | P | 1009 | P | 872.3 |
| | | clone_end = 3/gb = AI850953/gi = 5494859/ug = Mm 30849/len = 328/STRA = for | | | | | | | |
| 14 | 97549_at | Cluster Incl L29468 Mouse cofilin Isoform mRNA, complete cds/cds = (131,631)/gb = L29468/gi = 498016/ug = Mm 3373/ | 361.1 | P | 308 | P | 308.3 | P | 318.2 |
| | | len = 2974/STRA = for | | | | | | | |
| 14 | 99141_at | Cluster Incl U09816 Mus musculus GM2 activator protein (Gm2a) mRNA, complete cds/cds = (56,637)/gb = U09616/ | 355.8 | P | 746.2 | P | 253.7 | P | 736.7 |
| | | gi = 540017/ug = Mm 4140/len = 2003/STRA = for | | | | | | | |
| 14 | 99160_s_at | Cluster Incl AW227647: up12b06.y1 Mus musculus cDNA, 5 end/clone = IMAGE-2654099/clone_end = 5/gb = AW227647/ | 635.5 | P | 400.7 | P | 719.9 | P | 866.1 |
| | | gi = 6556943/ug = Mm 41665/len = 410/STRA = for | | | | | | | |
| 14 | 99642_i_at | Cluster Incl X61232: Mouse mRNA for carboxypeptidase H/cds = (39,1472)/gb = X61232/gi = 50312/ug = Mm 43603/ | 744.4 | P | 964.4 | P | 939.3 | P | 701.5 |
| | | len = 1992/STRA = for | | | | | | | |
| 14 | 99643_f_at | Cluster Incl X61232: Mouse mRNA for carboxypeptidase H/cds = (39,1472)/gb = X61232/gi = 50312/ug = Mm 43603/ | 719.8 | P | 1067.9 | P | 933.4 | P | 849.5 |
| | | len = 1992/STRA = for | | | | | | | |
| 14 | 100042_at | Cluster Incl AI837921 UI-M-AL0-abq-d-08-0-UI s1 Mus musculus cDNA, 3 end/clone = UI-M-AL0-abq-d-08-0-UI/ | 246.8 | P | 273.2 | P | 226.9 | P | 281 |
| | | clone_end = 3/gb = AI837921/gi = 5472134/ug = Mm 44240/len = 244/STRA = for | | | | | | | |
| 14 | 100120_at | Cluster Incl L17324 Nidogen/cds = (0,751)/gb = L17324/gi = 439608/ug = Mm 4691/len = 752/STRA = for | 336.8 | P | 549.6 | P | 438.3 | P | 373.9 |
| 14 | 100136_at | Cluster Incl M32017.Lysosomal membrane glycoprotein 2/cds = (0,692)/gb = M32017/gi = 198799/ug = Mm 486/len = 1067/ | 540.9 | P | 442.3 | P | 403.9 | P | 685.8 |
| | | STRA = for | | | | | | | |
| 14 | 100151_at | Cluster Incl L29441: Mus musculus mRNA, complete cds/cds = (69,1250)/gb = L29441/gi = 459889/ug = Mm 4962/len = 2029/ | 1020.8 | P | 1060.2 | P | 1156.9 | P | 1053.2 |
| | | STRA = for | | | | | | | |
| 14 | 100528_at | Cluster Incl AI852278 UI-M-BH0-aje-d-03-0-UI s1 Mus musculus cDNA, 3 end/clone = UI-M-BH0-aje-d-03-0-UI/ | 205.1 | P | 520.8 | P | 193.6 | P | 431 |
| | | clone_end = 3/gb = AI852278/gi = 5496184/ug = Mm 5203/len = 185/STRA = for | | | | | | | |
| 14 | 101008_at | Cluster Incl AB023485 Mus musculus mRNA for transcription factor CA150b, complete cds/cds = (4,3108)/gb = AB023485/ | 119.3 | P | 106.5 | P | 120.3 | P | 124.6 |
| | | gi = 6329165/ug = Mm 67998/len = 3526/STRA = for | | | | | | | |
| 14 | 101039_at | Cluster Incl X04647.Procollagen, type IV, alpha 2/cds = (0,2216)/gb = X04647/gi = 1022322/ug = Mm 6954/len = 3189/ | 1003.7 | P | 1254 | P | 1479.4 | P | 1430.7 |
| | | STRA = rev | | | | | | | |
| 14 | 102049_at | Cluster Incl AJ001418 Mus musculus mRNA for pyruvate dehydrogenase kinase-like protein/cds = (116,1354)/ | 246.3 | P | 149.3 | P | 247.7 | P | 197 |
| | | gb = AJ001418/gi = 3005989/ug = Mm.10283/len = 3449/STRA = for | | | | | | | |
| 14 | 102340_at | Cluster Incl AA833077 uc94g09.r1 Mus musculus cDNA, 5 end/clone = IMAGE-1433344/clone_end = 5/gb = AA833077/ | 109.9 | P | 108.7 | P | 78.3 | P | 109.3 |
| | | gi = 2906805/ug = Mm.11116/len = 379/STRA = for | | | | | | | |
| 14 | 102346_at | Cluster Incl AI747755 ul21h12.x1 Mus musculus cDNA, 3 end/clone = IMAGE-2088263/clone_end = 3/gb = AI747755/ | 122.9 | P | 62.4 | P | 128.7 | P | 106.4 |
| | | gi = 5126019/ug = Mm 11218/len = 431/STRA = for | | | | | | | |
| 14 | 102370_at | Cluster Incl AA822174: vp36a09.r1 Mus musculus cDNA, 5 end/clone = IMAGE-1078744/clone_end = 5/gb = AA822174/ | 93.3 | P | 192.5 | P | 242.7 | P | 191.8 |
| | | gi = 2892042/ug = Mm.1187/len = 329/STRA = for | | | | | | | |
| 14 | 102827_at | Cluster Incl AW124633 UI-M-BH2 1-apw-c-02-0-UI s1 Mus musculus cDNA, 3 end/clone = UI-M-BH2.1-apw-c-02-0-UI/ | 440.8 | P | 399.6 | P | 350.1 | P | 418.5 |
| | | clone_end = 3/gb = AW124633/gi = 6100163/ug = Mm.14478/len = 421/STRA = for | | | | | | | |
| 14 | 103015_at | Cluster Incl U41465: B-cell leukemia/lymphoma 6/cds = (100,2223)/gb = U41465/gi = 1209719/ug = Mm 15811/len = 2373/ | 252.6 | P | 355.6 | P | 433.1 | P | 316.3 |
| | | STRA = for | | | | | | | |
| 14 | 103338_at | Cluster Incl AW120504 UI-M-BH1-ami-f-11-0-UI s2 Mus musculus cDNA, 3 end/clone = UI-M-BH1-ami-f-11-0-UI/ | 119.4 | P | 93.6 | P | 97.8 | P | 84.2 |
| | | clone_end = 3/gb = AW120504/gi = 6095837/ug = Mm.18113/len = 336/STRA = for | | | | | | | |
| 14 | 103359_at | Cluster Incl AI854144 UI-M-BH0-ajw-g-04-0-UI s1 Mus musculus cDNA, 3 end/clone = UI-M-BH0-ajw-g-04-0-UI/ | 389.2 | P | 287.2 | P | 292.3 | P | 288.3 |
| | | clone_end = 3/gb = AI854144/gi = 5498050/ug = Mm 18460/len = 515/STRA = for | | | | | | | |
| 14 | 103414_at | Cluster Incl AF049850 Mus musculus major histocompatibility locus class III region- complemenl C4 (C4) and cytochrome | 344.4 | P | 408.5 | P | 552.7 | P | 302.3 |
| | | P450 hydroxylase A (CYP21OH-A) genes, complete cds, sip pseudogene, complete sequence, NG6, SKI, and complement | | | | | | | |
| | | factor B (Bf) genes, comp/STRA = for | | | | | | | |
| 14 | 103796_at | Cluster Incl AF064071: Mus musculus apoptotic protease activating factor 1 mRNA, complete cds/cds = (585,4301)/ | 166.1 | P | 261.5 | P | 149.3 | P | 161 |
| | | gb = AF064071/gi = 3694812/ug = Mm.20836/len = 5152/STRA = for | | | | | | | |
| 14 | 103893_at | Cluster Incl AW048781 UI-M-BH1-amg-b-03-0-UI s1 Mus musculus cDNA, 3 end/clone = UI-M-BH1-amg-b-03-0-UI/ | 463.4 | P | 349.5 | P | 319.5 | P | 474.9 |
| | | clone_end = 3/gb = AW048781/gi = 5909310/ug = Mm.21295/len = 404/STRA = for | | | | | | | |
| 14 | 103901_at | Cluster Incl AW213777 uo42e09 x1 Mus musculus cDNA, 3 end/clone = IMAGE-2645224/clone_end = 3/gb = AW213777/ | 140.6 | P | 181.5 | P | 139.5 | P | 138.8 |
| | | gi = 6520028/ug = Mm.21331/len = 313/STRA = for | | | | | | | |
| 14 | 104288_at | Cluster Incl AI853265 UI-M-BH0-aji-c-07-0-UI s1 Mus musculus cDNA, 3 end/clone = UI-M-BH0-aji-c-07-0-UI/clone_end = 3/ | 386.1 | P | 391.6 | P | 468.3 | P | 369.9 |
| | | gb = AI853265/gi = 5497171/ug = Mm 22276/len = 433/STRA = for | | | | | | | |
| 14 | 104744_at | Cluster Incl AW046775 UI-M-BH1-alt-h-01-0-UI s1 Mus musculus cDNA, 3 end/clone = UI-M-BH1-alt-h-01-0-UI/ | 264.1 | P | 240 | P | 216.6 | P | 246 |
| | | clone_end = 3/gb = AW046775/gi = 5907304/ug = Mm 24707/len = 174/STRA = for | | | | | | | |
| 14 | 104758_at | Cluster Incl AA895984: vx52h06.r1 Mus musculus cDNA, 5 end/clone = IMAGE-1278875/clone_end = 5/gb = AA895984/ | 178.4 | P | 192.5 | P | 239.2 | P | 145.7 |
| | | gi = 3032377/ug = Mm.24794/len = 339/STRA = for | | | | | | | |
| 14 | 94354_at | Cluster Incl AI845514: UI-M-AO1-aek-d-04-0-UI.s1 Mus musculus cDNA, 3 end/clone = UI-M-AO1-aek-d-04-0-UI/ | 374.1 | P | 410.4 | P | 286.4 | P | 429.6 |
| | | clone_end = 3/gb = AI845514/gi = 5489420/ug = Mm 25221/len = 409/STRA = for | | | | | | | |
| 14 | 94359_at | Cluster Incl AI849556 UI-M-AH1-agr-b-05-0-UI s1 Mus musculus cDNA, 3 end/clone = UI-M-AH1-agr-b-05-0-UI/ | 899 | P | 932.4 | P | 911.1 | P | 939.1 |
| | | clone_end = 3/gb = AI849556/gi = 5493462/ug = Mm 25248/len = 396/STRA = rev | | | | | | | |
| 14 | 94374_at | Cluster Incl AI850378 UI-M-BG1-aig-d-01-0-UI s1 Mus musculus cDNA, 3 end/clone = UI-M-BG1-aig-d-01-0-UI/ | 303.9 | P | 295.4 | P | 270.9 | P | 264.5 |
| | | clone_end = 3/gb = AI850378/gi = 5494284/ug = Mm.25445/len = 449/STRA = for | | | | | | | |
| 14 | 94420_f_at | Cluster Incl AB000777: Cryptochrome 1 (photolyase-like)/cds = (19,1839)/gb = AB000777/gi = 1816438/ug = Mm 26237/ | 190 | P | 259.4 | P | 250 | P | 278.3 |
| | | len = 2450/STRA = for | | | | | | | |
| 14 | 95033_at | Cluster Incl AW049513 UI-M-BH1-anf-b-11-0-UI s1 Mus musculus cDNA, 3 end/clone = UI-M-BH1-anf-b-11-0-UI/ | 311.4 | P | 354.4 | P | 257.5 | P | 326.8 |
| | | clone_end = 3/gb = AW049513/gi = 5910042/ug = Mm 27535/len = 508/STRA = for | | | | | | | |
| 14 | 95564_at | Cluster Incl AI117859 uc41d02 r1 Mus musculus cDNA, 5 end/clone = IMAGE-1400547/clone_end = 5/gb = AI117859/ | 122.7 | P | 119.3 | P | 103.9 | P | 148.7 |
| | | gi = 3518183/ug = Mm.27785/len = 328/STRA = for | | | | | | | |
| 14 | 95601_at | Cluster Incl AW125420 UI-M-BH2 3-aqh-f-01-0-UI s1 Mus musculus cDNA, 3 end/clone = UI-M-BH2 3-aqh-f-01-0-UI/ | 670.1 | P | 632.4 | P | 642.1 | P | 657.4 |
| | | clone_end = 3/gb = AW125420/gi = 6100950/ug = Mm 27937/len = 524/STRA = for | | | | | | | |
| 14 | 96221_at | Cluster Incl AI606300 vm55g05 y1 Mus musculus cDNA, 5 end/clone = IMAGE-1002200/clone_end = 5/gb = AI606300/ | 144.8 | P | 146.1 | P | 130.1 | P | 123.4 |
| | | gi = 4615467/ug = Mm 28710/len = 406/STRA = for | | | | | | | |
| 14 | 96774_at | Cluster Incl AW047139 UI-M-BH1-alu-c-08-0-UI s1 Mus musculus cDNA, 3 end/clone = UI-M-BH1-alu-c-08-0-UI/ | 703.9 | P | 576.5 | P | 569 | P | 561.1 |
| | | clone_end = 3/gb = AW047139/gi = 5907668/ug = Mm 29048/len = 363/STRA = for | | | | | | | |
| 14 | 97406_at | Cluster Incl AW122258 UI-M-BH2 2-aov-g-09-0-UI.s1 Mus musculus cDNA, 3 end/clone = UI-M-BH2.2-aov-g-09-0-UI/ | 297.5 | P | 325.4 | P | 339.5 | P | 288.3 |
| | | clone_end = 3/gb = AW122258/gi = 6097727/ug = Mm 29914/len = 285/STRA = rev | | | | | | | |
| 14 | 97985_f_at | Cluster Incl AA709672 vt35h09 r1 Mus musculus cDNA, 5 end/clone = IMAGE-1165121/clone_end = 5/gb = AA709672/ | 870.2 | P | 695.7 | P | 600.8 | P | 647.3 |
| | | gi = 2719590/ug = Mm.31297/len = 364/STRA = for | | | | | | | |
| 14 | 98478_at | Cluster Incl U95826 Mus musculus cyclin G2 mRNA, complete cds/cds = (123,1157)/gb = U95826/gi = 2149913/ | 286.7 | P | 334.1 | P | 291.9 | P | 303.1 |
| | | ug = Mm 3527/len = 1280/STRA = for | | | | | | | |
| 14 | 98505_i_at | Cluster Incl D16333 Mouse mRNA for coproporphyrinogen oxidase, complete cds/cds = (401,1465)/gb = D16333/gi = 436576/ | 365.1 | P | 382.4 | P | 311.9 | P | 379.4 |
| | | ug = Mm 35820/len = 3071/STRA = for | | | | | | | |
| 14 | 98506_r_at | Cluster Incl D16333 Mouse mRNA for coproporphyrinogen oxidase, complete cds/cds = (401,1465)/gb = D16333/gi = 436576/ | 189.9 | P | 263.4 | P | 179.8 | P | 258.5 |
| | | ug = Mm 35820/len = 3071/STRA = for | | | | | | | |
| 14 | 99064_at | Cluster Incl AF026469: Ubiquitous nuclear protein/cds = (50,2938)/gb = AF026469/gi = 2655203/ug = Mm 3974/len = 3329/ | 700 | P | 855.5 | P | 753.7 | P | 781.1 |
| | | STRA = for | | | | | | | |
| 14 | 99500_at | Cluster Incl U13174: Solute carrier family 12, member 2/cds = (129,3746)/gb = U13174/gi = 3885309/ug = Mm 4168/len = 6504/ | 400.6 | P | 474.6 | P | 409.8 | P | 477.5 |
| | | STRA = rev | | | | | | | |
| 14 | 99532_at | Cluster Incl D78382: Mouse DNA for tob family, complete cds/cds = (0,1091)/gb = D78382/gi = 1469158/ug = Mm 4218/ | 893.3 | P | 1102.1 | P | 1301.1 | P | 817.8 |
| | | len = 1092/STRA = for | | | | | | | |
| 14 | 99960_at | Cluster Incl U18310 SAPK/Erk/kinase 1/cds = (28,1221)/gb = U18310/gi = 1754530/ug = Mm 4412/len = 2292/STRA = for | 246.6 | P | 218.6 | P | 340.3 | P | 302.7 |
| 14 | 100034_at | Cluster Incl U54705: Mus musculus tumor supressor maspin mRNA, complete cds/cds = (189,1316)/gb = U54705/ | 1118.3 | P | 1154.3 | P | 954.6 | P | 1064.8 |
| | | gi = 1490512/ug = Mm 4622/len = 1432/STRA = for | | | | | | | |
| 14 | 100475_at | Cluster Incl D63902: Zinc finger protein 147/cds = (9,1913)/gb = D63902/gi = 1088466/ug = Mm.4973/len = 5475/STRA = rev | 103.6 | P | 185.3 | P | 260.4 | P | 262.6 |
| 14 | 100482_at | Cluster Incl AI836408 UI-M-APO-abi-g-08-0-UI s1 Mus musculus cDNA, 3 end/clone = UI-M-AP0-abi-g-08-0-UI/ | 782.4 | P | 908 | P | 799.7 | P | 732 |
| | | clone_end = 3/gb = AI836408/gi = 5470621/ug = Mm 5018/len = 277/STRA = rev | | | | | | | |
| 14 | 100978_at | Cluster Incl U62105 FSHD region gene 1/cds = (116,892)/gb = U62105/gi = 1762524/ug = Mm 67/len = 1022/STRA = for | 247.1 | P | 269.8 | P | 267.9 | P | 210.9 |
| 14 | 102870_at | Cluster Incl AW125272: UI-M-BH2 1-aps-h-06-0-UI s1 Mus musculus cDNA, 3 end/clone = UI-M-BH2 1-aps-h-06-0-UI/ | 290.5 | P | 234.6 | P | 333.5 | P | 322.4 |
| | | clone_end = 3/gb = AW125272/gi = 6100802/ug = Mm 14787/len = 349/STRA = for | | | | | | | |
| 14 | 102912_at | Cluster Incl AI265115 uk03d02 y1 Mus musculus cDNA, 5 end/clone = IMAGE-1958307/clone_end = 5/gb = AI265115/ | 348 | P | 497.7 | P | 481 | P | 371.7 |
| | | gi = 3883273/ug = Mm 1610/len = 473/STRA = for | | | | | | | |
| 14 | 103263_at | Cluster Incl AF079755 Mus musculus enhancer of polycomb (Epc1) mRNA, complete cds/cds = (220,2511)/gb = AF079765/ | 470 | P | 416 | P | 361.2 | P | 379.5 |
| | | gi = 3757891/ug = Mm 20828/len = 3895/STRA = for | | | | | | | |
| 14 | 103456_at | Cluster Incl AI841800 UI-M-AN0-acl-g-06-0-UI s1 Mus musculus cDNA, 3 end/clone = UI-M-AN0-acl-g-06-0-UI/ | 536.8 | P | 592.1 | P | 644.8 | P | 523.4 |
| | | clone_end = 3/gb = AI841800/gi = 5476013/ug = Mm 21657/len = 371/STRA = for | | | | | | | |
| 14 | 103457_at | Cluster Incl AF083464 Mus musculus DNA polymerase zeta catalytic subunit mRNA, complete cds/cds = (48,9416)/ | 237.7 | P | 219.3 | P | 167.2 | P | 151.5 |
| | | gb = AF083464/gi = 4079830/ug = Mm.2167/len = 10404/STRA = for | | | | | | | |
| 14 | 103697_at | Cluster Incl AW061234 UI-M-BH1-amj-b-11-0-UI s2 Mus musculus cDNA, 3 end/clone = UI-M-BH1-emj-b-11-0-UI/ | 281.5 | P | 343.3 | P | 403.6 | P | 314.9 |
| | | clone_end = 3/gb = AW061234/gi = 6008985/ug = Mm 24003/len = 462/STRA = for | | | | | | | |
| 14 | 103773_at | Cluster Incl AW047874 UI-M-BH1-ala-g-02-0-UI s1 Mus musculus cDNA, 3 end/clone = UI-M-BH1-als-g-02-0-UI/ | 223.8 | P | 269.3 | P | 266.8 | P | 176 |
| | | clone_end = 3/gb = AW047874/gi = 5908403/ug = Mm.24787/len = 433/STRA = for | | | | | | | |
| 14 | 104033_at | Cluster Incl AI841996 UI-M-AH0-ada-b-03-0-UI s1 Mus musculus cDNA, 3 end/clone = UI-M-AH0-ada-b-03-0-UI/ | 270.5 | P | 269.1 | P | 235.3 | P | 295.7 |
| | | clone_end = 3/gb = AI841996/gi = 5476209/ug = Mm 27051/len = 401/STRA = for | | | | | | | |
| 14 | 104189_at | Cluster Incl AI122079 uc46e01.r1 Mus musculus cDNA, 5 end/clone = IMAGE-1401048/clone_end = 5/gb = AI122079/ | 193.6 | P | 211.7 | P | 245 | P | 232.2 |
| | | gi = 3522403/ug = Mm 27463/len = 362/STRA = for | | | | | | | |
| 14 | 104403_at | Cluster Incl AI854118 UI-M-BH0-ajw-d-12-0-UI s1 Mus musculus cDNA, 3 end/clone = UI-M-BH0-ajw-d-12-0-UI/ | 302.2 | P | 398 | P | 328.2 | P | 321.5 |
| | | clone_end = 3/gb = AI854118/gi = 5498024/ug = Mm 28691/len = 504/STRA = rev | | | | | | | |
| 14 | 104513_at | Cluster Incl AA688938: vs04f09.r1 Mus musculus cDNA, 5 end/clone = IMAGE-1137257/clone_end = 5/gb = AA688938/ | 192.8 | P | 168.4 | P | 248.2 | P | 252.9 |
| | | gi = 2678367/ug = Mm 3090/len = 197/STRA = for | | | | | | | |
| 14 | 92217_s_at | Cluster Incl U05265 Glycoprotein 49 B/cds = (43,1050)/gb = U05265/gi = 475446/ug = Mm 34408/len = 1420/STRA = for | 308.8 | P | 359.3 | P | 316.7 | P | 467 |
| 14 | 92384_at | Cluster Incl X74351: Xeroderma pigmentosum, complementation group A/cds = (91,909)/gb = X74351/gi = 440584/ | 117.5 | P | 142.4 | P | 153 | P | 136.1 |
| | | ug = Mm.4049/len = 938/STRA = for | | | | | | | |
| 14 | 92791_at | Cluster Incl AA518588 vh94g10 r1 Mus musculus cDNA, 5 end/clone = IMAGE-901986/clone_end = 5/gb = AA518586/ | 393.8 | P | 332.4 | P | 378.6 | P | 311.5 |
| | | gi = 2258612/ug = Mm. 45985/len = 255/STRA = rev | | | | | | | |
| 14 | 93626_at | Cluster Incl AF103875: Mus musculus placenta-specific ATP-binding cassette transporter (Abcp) mRNA, partial cds/ | 177.5 | P | 229.5 | P | 200.5 | P | 179.6 |
| | | cds = (0,443)/gb = AF103875/gi = 4185797/ug = Mm.7458/len = 502/STRA = for | | | | | | | |
| 14 | 95387_f_at | Cluster Incl AA266467: mz67g09 r1 Mus musculus cDNA, 5 end/clone = IMAGE-718528/clone_end = 5/gb = AA266467/ | 318.4 | P | 328.7 | P | 297.5 | P | 280.1 |
| | | gi = 1902637/ug = Mm 23470/len = 372/STRA = rev | | | | | | | |
| 14 | 95897_at | Cluster Incl AW061228 UI-M-BH1-amj-a-12-0-UI s2 Mus musculus cDNA, 3 end/clone = UI-M-BH1-amj-a-12-0-UI/ | 260.3 | P | 201.6 | P | 196.7 | P | 211.4 |
| | | clone_end = 3/gb = AW061228/gi = 6008979/ug = Mm.24179/len = 475/STRA = rev | | | | | | | |
| 14 | 98855_r_at | Cluster Incl AA014745 mh18b05 r1 Mus musculus cDNA, 5 end/clone = IMAGE-442833/clone_end = 5/gb = AA014745/ | 129.1 | P | 182.2 | P | 209.3 | P | 100 |
| | | gi = 1475626/ug = Mm 5096/len = 189/STRA = for | | | | | | | |
| 14 | 95785_s_at | Cluster Incl Y13361 RAB7, member RAS oncogene family, pseudogene 1/cds = (0,623)/gb = Y13361/gi = 2168154/ | 366.1 | P | 297.5 | P | 413.3 | P | 477.6 |
| | | ug = Mm 87807/len = 624/STRA = for | | | | | | | |
| 14 | 101221_at | Cluster Incl C76746 C76746 Mus musculus cDNA, 3 end/clone = J0018D06/clone_end = 3/gb = C76746/gi = 2517076/ | 145.6 | P | 207.9 | P | 190.3 | P | 210.7 |
| | | ug = Mm.5529/len = 574/STRA = rev | | | | | | | |
| 14 | 93836_at | Cluster Incl AF041054 Mus musculus E1B 19K/Bcl-2-binding protein homolog (Nip3) mRNA, nuclear gene encoding | 195.3 | P | 224.7 | P | 223.2 | P | 251.3 |
| | | mitochondrial protein, complete cds/cds = (83,646)/gb = AF041054/gi = 2773344/ug = Mm 2159/len = 1311/STRA = for | | | | | | | |
| 14 | 96106_at | Cluster Incl AW049122 UI-M-BH1-amt-h-11-0-UI s1 Mus musculus cDNA, 3 end/clone = UI-M-BH1-amt-h-11-0-UI/ | 114.2 | P | 173.6 | P | 145.6 | P | 169.4 |
| | | clone_end = 3/gb = AW049122/gi = 5909651/ug = Mm 26903/len = 513/STRA = for | | | | | | | |
| 14 | 96679_at | Cluster Incl AW120711: UI-M-BH2 3-anz-e-10-0-UI.s1 Mus musculus cDNA, 3 end/clone = UI-M-BH2.3-anz-e-10-0-UI/ | 44.6 | P | 32.7 | A | 71.1 | P | 23 |
| | | clone_end = 3/gb = AW120711/gi = 6096009/ug = Mm.27432/len = 497/STRA = for | | | | | | | |
| 14 | 96884_at | Cluster Incl AI847631 UI-M-AP1-agg-e-12-0-UI s1 Mus musculus cDNA, 3 end/clone = UI-M-AP1-agg-e-12-0-UI/ | 382 | A | 437.2 | P | 422.5 | P | 472 |
| | | clone_end = 3/gb = AI847631/gi = 5491537/ug = Mm.30081/len = 334/STRA = for | | | | | | | |
| 14 | 97475_at | Cluster Incl D38517.Mouse mRNA for Dhm1 protein, complete cds/cds = (0,2843)/gb = D38517/gi = 1060920/ug = Mm 3065/ | 57 | A | 83.4 | P | 54 | P | 94.3 |
| | | len = 2844/STRA = for | | | | | | | |
| 14 | 103673_at | Cluster Incl M57891: Complement component 2 (wthin H-2S)/cds = (52,2334)/gb = M57891/gi = 192436/ug = Mm 2081/ | 196.9 | P | 198.4 | P | 166.7 | P | 162.6 |
| | | len = 2587/STRA = for | | | | | | | |
| 14 | 104569_at | Cluster Incl AW122024 UI-M-BH2.2-aoo-a-11-0-UI s1 Mus musculus cDNA, 3 end/clone = UI-M-BH2 2-aoo-a-11-0-UI/ | 174.4 | P | 154.4 | P | 145 | P | 137.5 |
| | | clone_end = 3/gb = AW122024/gi = 6097430/ug = Mm 2389/len = 498/STRA = for | | | | | | | |
| 14 | 104600_at | Cluster Incl AI841692 UI-M-AL0-abo-a-02-0-UI s1 Mus musculus cDNA, 3 end/clone = UI-M-AL0-abo-a-02-0-UI/ | 71.1 | P | 66.1 | P | 56.8 | M | 72.8 |
| | | clone_end = 3/gb = AI841692/gi = 5475905/ug = Mm 24084/len = 415/STRA = for | | | | | | | |
| 14 | 95031_at | Cluster Incl AW060212: UI-M-BH1-amu-f-06-0-UI.s1 Mus musculus cDNA, 3 end/clone = UI-M-BH1-amu-f-06-0-UI/ | 31.6 | P | 75.3 | P | 46.4 | P | 53.4 |
| | | clone_end = 3/gb = AW060212/gi = 6007963/ug = Mm 27520/len = 378/STRA = for | | | | | | | |
| 14 | 97972_at | Cluster Incl AB012161 Mus musculus gene for KF-1, complete cds/cds = (0,2051)/gb = AB012161/gi = 4521316/ug = Mm 310/ | 126.1 | P | 142.2 | P | 163.7 | P | 111.1 |
| | | len = 2052/STRA = for | | | | | | | |
| 14 | 99018_at | Cluster Incl AA874446 vx09g08 r1 Mus musculus cDNA, 5 end/clone = IMAGE-1263998/clone_end = 5/gb = AA874446/ | 64.8 | P | 74.3 | P | 43.6 | A | 71.8 |
| | | gi = 2979135/ug = Mm 385/len = 511/STRA = for | | | | | | | |
| 14 | 99512_at | Cluster Incl AI852647: UI-M-BH0-aiu-h-07-0-UI s1 Mus musculus cDNA, 3 end/clone = UI-M-BH0-aiu-h-07-0-UI/ | 550.3 | P | 621.9 | A | 517.2 | P | 522.7 |
| | | clone_end = 3/gb = AI852647/gi = 5496553/ug = Mm 41858/len = 379/STRA = for | | | | | | | |
| 14 | 103678_at | Cluster Incl AI841139 UI-M-AH0-acz-g-08-0-UI s1 Mus musculus cDNA, 3 end/clone = UI-M-AH0-acz-g-08-0-UI/ | 233.6 | P | 225.5 | P | 206.6 | P | 252.2 |
| | | clone_end = 3/gb = AI841139/gi = 5475352/ug = Mm 23879/len = 450/STRA = for | | | | | | | |
| 14 | 104671_at | Cluster Incl D88994 AMP deaminase 3/cds = (5,2083)/gb = D88994/gi = 3493348/ug = Mm 3238/len = 2127/STRA = for | 59.1 | P | 82.2 | P | 164.9 | P | 133 |
| 14 | 93148_at | Cluster Incl AI851013 UI-M-BH0-ajv-c-07-0-UI s1 Mus musculus cDNA, 3 end/clone = UI-M-BH0-ajv-c-07-0-UI/ | 183.3 | P | 159.7 | P | 156.8 | P | 166.1 |
| | | clone_end = 3/gb = AI851013/gi = 5494919/ug = Mm 5231/len = 485/STRA = for | | | | | | | |
| 14 | 98802_at | Cluster Incl D30782 Mouse mRNA for epiregulin, complete cds/cds = (138,626)/gb = D30782/gi = 1255631/ug = Mm 4791/ | 27.7 | A | 59.6 | P | 91.9 | P | 72.1 |
| | | len = 4141/STRA = for | | | | | | | |
| 14 | 94066_at | Cluster Incl AW123517 UI-M-BH2 1-aqb-c-09-0-UI s1 Mus musculus cDNA, 3 end/clone = UI-M-BH2 1-aqb-c-09-0-UI/ | 278.7 | P | 298.1 | P | 345.3 | P | 311.2 |
| | | clone_end = 3/gb = AW123517/gi = 6099047/ug = Mm 22086/len = 244/STRA = for | | | | | | | |
| 14 | 95093_at | Cluster Incl AI035334 ue18f10x1 Mus musculus cDNA, 3 end/clone = IMAGE-1480747/clone_end = 3/gb = AI035334/ | 175.2 | P | 179.1 | P | 181.2 | P | 212.8 |
| | | gi = 3259044/ug = Mm.29303/len = 182/STRA = for | | | | | | | |
| 14 | 103011_at | Cluster Incl L36831: Transcriptional regulator, SIN3 yeast homolog A/cds = (78,3926)/gb = L36831/gi = 556218/ | 345.2 | P | 382.4 | A | 291.9 | P | 362.4 |
| | | ug = Mm 15755/len = 4721/STRA = for | | | | | | | |
| 14 | 101370_at | Cluster Incl U20619.Mus musculus serine-rich RNA polymerase suppressor protein 1 (SRP1) mRNA, complete sequence/ | 279.4 | P | 274.8 | P | 214.7 | P | 222.1 |
| | | cds = UNKNOWN/gb = U20619/gi = 3046878/ug = Mm 6952/len = 3882/STRA = for | | | | | | | |
| 14 | 102916_s_at | Cluster Incl AB010266 Mus musculus Creb-rp, Tnx and Cyp21 genes for cAMP response element binding protein-related | 116.3 | A | 874 | P | 172.2 | P | 418 |
| | | protein, lenascin-X and steroid 21-hydroxylase, partial cds and complete sequences/cds = (0,12344)/gb = AB010266/ | | | | | | | |
| | | gi = 2804287/ug = Mm.16163/len = /STRA = for | | | | | | | |
| 14 | 102922_at | Cluster Incl AI851387.UI-M-BH0-akh-g-12-0-UI s1 Mus musculus cDNA, 3 end/clone = UI-M-BH0-akh-g-12-0-UI/ | 69.9 | P | 36.5 | P | 37.5 | M | 97.3 |
| | | clone_end = 3/gb = AI851387/gi = 5495293/ug = Mm 1630/len = 436/STRA = rev | | | | | | | |
| 14 | 102966_at | Cluster Incl AA934166 vt48h11.r1 Mus musculus cDNA, 5 end/clone = IMAGE-1166373/clone_end = 5/gb = AA934166/ | 234.6 | P | 189.9 | P | 164.4 | M | 146.9 |
| | | gi = 3091323/ug = Mm 1819/len = 321/STRA = for | | | | | | | |
| 14 | 103254_at | Cluster Incl AW049897: UI-M-BH1-anr-h-08-0-UI s1 Mus musculus cDNA, 3 end/clone = UI-M-BH1-anr-h-08-0-UI/ | 183.4 | M | 118.9 | P | 168.5 | P | 225.8 |
| | | clone_end = 3/gb = AW049897/gi = 5910426/ug = Mm 20488/len = 275/STRA = for | | | | | | | |
| 14 | 104661_at | Cluster Incl AF031164: AF031164 Mus musculus cDNA/gb = AF031164/gi = 4887815/ug = Mm.31787/len = 800/STRA = for | 118.7 | P | 103.7 | P | 83.7 | A | 86.4 |
| 14 | 93846_at | Cluster Incl U82324 Mus musculus A6 protein tyrosine kinase homolog mRNA, complete cds/cds = (56,1108)/gb = U82324/ | 92.4 | P | 98.3 | P | 116.7 | P | 91.6 |
| | | gi = 1769576/ug = Mm 7958/len = 1200/STRA = for | | | | | | | |
| 14 | 97172_s_at | Cluster Incl D86037: Sulfonylurea receptor 2/cds = (161,4801)/gb = D86037/gi = 4996851/ug = Mm 35070/len = 6210/ | 34.8 | P | 32.5 | A | 62.2 | P | 43.3 |
| | | STRA = for | | | | | | | |
| 14 | 96692_at | Cluster Incl AA759408 vw64c05.r1 Mus musculus cDNA, 5 end/clone = IMAGE-1248584/clone_end = 5/gb = AA759408/ | 264.4 | P | 303.7 | P | 153.1 | A | 152.5 |
| | | gi = 2807202/ug = Mm.27523/len = 424/STRA = for | | | | | | | |
| 14 | 101538_i_at | Cluster Incl AW226939 um64a07.y1 Mus musculus cDNA, 5 end/clone = IMAGE-2286132/clone_end = 5/gb = AW226939/ | 249.1 | P | 164.3 | A | 198.4 | A | 211.3 |
| | | gi = 6556235/ug = Mm 88078/len = 486/STRA = rev | | | | | | | |
| 14 | 102836_at | Cluster Incl U96724: Putative phosphatase/cds = (111,1517)/gb = U96724/gi = 2121245/ug = Mm 1458/len = 2684/STRA = for | 155.6 | A | 195.6 | P | 281.7 | P | 248.3 |
| 14 | 94980_at | Cluster Incl AI006319: ua70b05.r1 Mus musculus cDNA, 5 end/clone = IMAGE-1362801/clone_end = 5/gb = AI006319/ | 18 | P | 133.4 | P | 159.5 | P | 148.9 |
| | | gi = 3215928/ug = Mm.27275/len = 381/STRA = for | | | | | | | |
| 14 | 103716_at | Cluster Incl AI851218: UI-M-BH0-ajx-g-06-0-UI s1 Mus musculus cDNA, 3 end/clone = UI-M-BH0-ajx-g-06-0-UI/ | 171.7 | P | 132 | P | 45.3 | A | 115.2 |
| | | clone_end = 3/gb = AI851218/gi = 5495124/ug = Mm.24160/len = 306/STRA = rev | | | | | | | |
| 14 | 103967_at | Cluster Incl AI551105 vx37f03 x1 Mus musculus cDNA, 3 end/clone = IMAGE-1277405/clone_end = 3/gb = AI551105/ | 66 | P | 61.4 | P | 42.9 | A | 53.4 |
| | | gi = 4483468/ug = Mm.25678/len = 390/STRA = for | | | | | | | |
| 14 | 95292_at | Cluster Incl AA189389 mt85f02 r1 Mus musculus cDNA, 5 end/clone = IMAGE-636699/clone_end = 5/gb = AA189389/ | 86.8 | P | 68.5 | P | 58.8 | P | 57.5 |
| | | gi = 1776990/ug = Mm 1919/len = 451/STRA = for | | | | | | | |
| 14 | 97253_at | Cluster Incl AI854343 UI-M-BG1-aic-c-07-0-UI s1 Mus musculus cDNA, 3 end/clone = UI-M-BG1-aic-c-07-0-UI/ | 124.7 | M | 125.7 | A | 146 | P | 168.5 |
| | | clone_end = 3/gb = AI854343/gi = 5498249/ug = Mm 27872/len = 305/STRA = for | | | | | | | |
| 14 | 102032_at | Cluster Incl AW060819: UI-M-BH1-amx-b-03-0-UI.s1 Mus musculus cDNA, 3 end/clone = UI-M-BH1-amx-b-03-0-UI/ | 212 | P | 258.7 | A | 313.7 | P | 232.4 |
| | | clone_end = 3/gb = AW060819/gi = 6008570/ug = Mm 10153/len = 507/STRA = for | | | | | | | |
| 14 | 102780_at | Cluster Incl Z31362: M musculus (Balb/C) Tx01 mRNA/cds = (0,159)/gb = Z31362/gi = 467582/ug = Mm 1364/len = 228/ | 181.9 | A | 194.2 | A | 219.5 | P | 204 |
| | | STRA = for | | | | | | | |
| 14 | 102852_at | Cluster Incl M31131: Cadhenn 2/cds = (332,3052)/gb = M31131/gi = 192327/ug = Mm 14897/len = 4321/STRA = for | 41.2 | A | 36.4 | P | 48.2 | A | 43.3 |
| 14 | 104065_at | Cluster Incl AW212878 uo68g01 x1 Mus musculus cDNA, 3 end/clone = IMAGE-2647728/clone_end = 3/gb = AW212878/ | 51.3 | P | 26.2 | A | 49.7 | P | 65.6 |
| | | gi = 6519026/ug = Mm 21596/len = 280/STRA = for | | | | | | | |
| 14 | 104067_at | Cluster Incl AI648999 uk33h10.x1 Mus musculus cDNA, 3 end/clone = IMAGE-1970851/clone_end = 3/gb = AI648999/ | 79.9 | P | 83.9 | A | 78.8 | P | 115.5 |
| | | gi = 4729833/ug = Mm.21608/len = 579/STRA = for | | | | | | | |
| 14 | 104144_at | Cluster Incl AW124369 UI-M-BH2 1-ape-a-04-0-UI.s1 Mus musculus cDNA, 3 end/clone = UI-M-BH2 1-ape-a-04-0-UI/ | 146.7 | P | 166 | P | 294.8 | P | 116.5 |
| | | clone_end = 3/gb = AW124369/gi = 6099899/ug = Mm 22147/len = 214/STRA = for | | | | | | | |
| 14 | 97984_i_at | Cluster Incl AA709672: vt35h09 r1 Mus musculus cDNA, 5 end/clone = IMAGE-1165121/clone_end = 5/gb = AA709672/ | 105.4 | P | 35.2 | A | 29.6 | M | 197.9 |
| | | gi = 2719590/ug = Mm.31297/len = 364/STRA = for | | | | | | | |
| 14 | 104215_at | Cluster Incl AA592069.vo14b08.r1 Mus musculus cDNA, 5 end/clone = IMAGE-1049847/clone_end = 5/gb = AA592069/ | 35.3 | M | 30.9 | A | 74.3 | P | 38.7 |
| | | gi = 2405732/ug = Mm.27843/len = 329/STRA = for | | | | | | | |
| 14 | 98297_at | Cluster Incl AF110818 Mus musculus orphan G protein-coupled receptor FEX mRNA, complete cds/cds = (0,2723)/ | 168.1 | P | 115.3 | A | 2.3 | A | 120.5 |
| | | gb = AF110818/gi = 4262545/ug = Mm.42103/len = 3115/STRA = for | | | | | | | |
| 14 | 93054_at | Cluster Incl AI846368 UI-M-AN1-afa-e-09-0-UI s1 Mus musculus cDNA, 3 end/clone = UI-M-AN1-afa-e-09-0-UI/ | 109.9 | P | 197.1 | P | 135.6 | P | 129.4 |
| | | clone_end = 3/gb = AI846368/gi = 5490274/ug = Mm 15351/len = 385/STRA = for | | | | | | | |
| 14 | 94324_f_at | Cluster Incl U49878.3-hydroxy-3-methylglutaryl-coenzyme A lyase/cds = (34,1011)/gb = U49878/gi = 1292951/ | 522.8 | P | 447 | A | 455.1 | A | 441.7 |
| | | ug = Mm 22668/len = 1440/STRA = for | | | | | | | |
| 14 | 96912_s_at | Cluster Incl X15591 Cytotoxic T lymphocyte-associated protein 2 alpha/cds = (6,416)/gb = X15591/gi = 50588/ug = Mm.30144/ | 208.7 | M | 216.8 | A | 333.4 | P | 446.9 |
| | | len = 664/STRA = for | | | | | | | |
| 14 | 99166_at | Cluster Incl AI845963 UI-M-AP1-agl-a-09-0-UI s1 Mus musculus cDNA, 3 end/clone = UI-M-AP1-agl-a-09-0-UI/ | 247.8 | P | 341.2 | P | 322.6 | P | 327.6 |
| | | clone_end = 3/gb = AI845963/gi = 5489869/ug = Mm 41683/len = 223/STRA = for | | | | | | | |
| 14 | 96811_at | Cluster Incl AW049806 UI-M-BH1-anm-g-08-0-UI s1 Mus musculus cDNA, 3 end/clone = UI-M-BH1-anm-g-08-0-UI/ | 177.8 | P | 121.8 | P | 98.7 | A | 145.3 |
| | | clone_end = 3/gb = AW04986/gi = 5910335/ug = Mm 29274/len = 457/STRA = for | | | | | | | |
| 14 | 99510_at | Cluster Incl X59274 M musculus mRNA brain protein kinase C beta-1/cds = UNKNOWN/gb = X59274/gi = 50185/ | 155.5 | P | 169.3 | P | 184.8 | P | 173.1 |
| | | ug = Mm 4182/len = 2493/STRA = for | | | | | | | |
| 14 | 101848_g_at | Cluster Incl AF040242 untitled/cds = (0,240)/gb = AF040242/gi = 3309551/ug = Mm 8551/len = 241/STRA = for | 186.1 | P | 120 | P | 113.5 | A | 124.1 |
| 14 | 100321_f_at | Cluster Incl AA407120 EST02104 Mus musculus cDNA, 5 end/clone = C0017F03/clone_end = 5/gb = AA407120/ | 19.6 | A | 40.3 | P | 35.5 | P | 8.5 |
| | | gi = 2067019/ug = Mm 87656/len = 359/STRA = for | | | | | | | |
| 14 | 101804_at | Cluster Incl AF032715 Mus musculus type II peroxiredoxin protein 3 (PrxII-3) pseudogene, complete sequence/ | 182.1 | P | 244.3 | A | 273.1 | P | 137.8 |
| | | cds = (0,452)/gb = AF032715/gi = 3603232/ug = Mm.57146/len = 453/STRA = for | | | | | | | |
| 14 | 94491_at | Cluster Incl AW123194: UI-M-BH2 1-apg-c-01-0-UI s1 Mus musculus cDNA, 3 end/clone = UI-M-BH2.1-apg-c-01-0-UI/ | 97.7 | A | 108.4 | A | 138.9 | A | 79.3 |
| | | clone_end = 3/gb = AW123194/gi = 6098764/ug = Mm 28912/len = 509/STRA = for | | | | | | | |
| 14 | 95094_g_at | Cluster Incl AI035334 ue18f10 x1 Mus musculus cDNA, 3 end/clone = IMAGE-1480747/clone_end = 3/gb = AI035334/ | 61.8 | M | 64.4 | A | 54.8 | P | 71.3 |
| | | gi = 3259044/ug = Mm.29303/len = 182/STRA = for | | | | | | | |
| 14 | 104368_at | Cluster Incl U51204: Mus musculus APC-binding protein EB2 mRNA, partial cds/cds = (0,677)/gb = U51204/gi = 1256435/ | 98.7 | M | 156.8 | P | 72.7 | A | 137.2 |
| | | ug = Mm 22628/len = 1540/STRA = for | | | | | | | |
| 14 | 96770_at | Cluster Incl AA607761 vo08f08 r1 Mus musculus cDNA, 5 end/clone = IMAGE-1040871/clone_end = 5/gb = AA607761/ | 8.7 | A | 62.4 | A | −151 | A | 136.7 |
| | | gi = 2455196/ug = Mm 29017/len = 491/STRA = for | | | | | | | |
| 14 | 100507_at | Cluster Incl Y09257 M musculus mRNA for NOV protein/cds = (32,1096)/gb = Y09257/gi = 1666472/ug = Mm 5167/len = 2364/ | 179.7 | P | 318.8 | P | 138.9 | A | 38.9 |
| | | STRA = for | | | | | | | |
| 14 | 102965_at | Cluster Incl AW121646 UI-M-BH2 3-aoc-b-03-0-UI s1 Mus musculus cDNA, 3 end/clone = UI-M-BH2 3-aoc-b-03-0-UI/ | 368.5 | M | 330.3 | P | 281.9 | A | 322 |
| | | clone_end = 3/gb = AW121646/gi = 6096979/ug = Mm.1806/len = 512/STRA = for | | | | | | | |
| 14 | 93609_at | Cluster Incl AA797709 vw31c03 r1 Mus musculus cDNA, 5 end/clone = IMAGE-1245412/clone_end = 5/gb = AA797709/ | 6.6 | A | 33.8 | A | 51.2 | P | 68.6 |
| | | gi = 2860664/ug = Mm 7265/len = 464/STRA = for | | | | | | | |
| 14 | 96528_at | Cluster Incl D18912 MUSGS01097 Mus musculus cDNA, 3 end/clone = mc0837/clone_end = 3/gb = D18912/gi = 1089548/ | 40.4 | P | 46.3 | A | 32.3 | M | 23.9 |
| | | ug = Mm 27078/len = 254/STRA = rev | | | | | | | |
| 14 | 97710_f_at | Cluster Incl AI425990: mg38e07 x1 Mus musculus cDNA, 3 end/clone = IMAGE-426084/clone_end = 3/gb = AI425990/ | 38.9 | A | 39.6 | P | 26.9 | P | −2.6 |
| | | gi = 4271921/ug = Mm 38264/len = 407/STRA = rev | | | | | | | |
| 15 | AFFX- | E Coli BioD 3 prime | 3823.4 | P | 3647.4 | P | 3538.3 | P | 3664.8 |
| | BioDn-3_at | | | | | | | | |
| 15 | AFFX-CreX- | Cre 5 prime | 5652.2 | P | 4849.7 | P | 5253.2 | P | 5281 |
| | 5_at | | | | | | | | |
| 15 | AFFX-CreX- | Cre 3 prime | 3655.4 | P | 3172.1 | P | 3425.1 | P | 3572.6 |
| | 3_at | | | | | | | | |
| 15 | AFFX-b- | M12481 Mouse cytoplasmic beta-actin mRNA (_5, _M, _3 represent transcript regions 5 prime, | 3966.2 | P | 2984.1 | P | 3332.6 | P | 3283.3 |
| | ActinMur/ | Middle, and 3 prime respectively) | | | | | | | |
| | M12481_5_at | | | | | | | | |
| 15 | AFFX-b- | M12481 Mouse cytoplasmic beta-actin mRNA (_5, _M, _3 represent transcript regions 5 prime, | 4665.2 | P | 4136.1 | P | 4477.5 | P | 4109.7 |
| | ActinMur/ | Middle, and 3 prime respectively) | | | | | | | |
| | M12481_{—} | | | | | | | | |
| | M_at | | | | | | | | |
| 15 | AFFX-b- | M12481 Mouse cytoplasmic beta-actin mRNA (_5, _M, _3 represent transcript regions 5 prime, | 3567.4 | P | 3594.7 | P | 3603.4 | P | 2794.9 |
| | ActinMur/ | Middle, and 3 prime respectively) | | | | | | | |
| | M12481_3_at | | | | | | | | |
| 15 | AFFX- | M32599 Mouse glyceraldehyde-3-phosphate dehydrogenase mRNA, complete cds (_5, _M, _3 represent | 5267.5 | P | 4395.7 | P | 4380.5 | P | 4606.7 |
| | GapdhMur/ | transcript regions 5 prime, Middle, and 3 prime respectively) | | | | | | | |
| | M32599_5_at | | | | | | | | |
| 15 | AFFX- | M32599 Mouse glyceraldehyde-3-phosphate dehydrogenase mRNA, complete cds (_5, _M, _3 represent | 4869.6 | P | 4367.5 | P | 4392.8 | P | 4421.4 |
| | GapdhMur/ | transcript regions 5 prime, Middle, and 3 prime respectively) | | | | | | | |
| | M32599_{—} | | | | | | | | |
| | M_at | | | | | | | | |
| 15 | AFFX- | M32599 Mouse glyceraldehyde-3-phosphate dehydrogenase mRNA, complete cds (_5, _M, _3 represent | 4317 | P | 3011.1 | P | 3064.9 | P | 3348.2 |
| | GapdhMur/ | transcript regions 5 prime, Middle, and 3 prime respectively) | | | | | | | |
| | M32599_3_at | | | | | | | | |
| 15 | AFFX-MUR_{—} | X63136 M musculus DNA for intragenic sequence Including B2 element | 2290.5 | P | 2296.1 | P | 2241.8 | P | 2251 |
| | b2_at | | | | | | | | |
| 15 | AFFX- | U01310 Mus musculus C57/Black6 BC1 scRNA | 4059.3 | P | 3571.3 | P | 3617.1 | P | 4202.9 |
| | MURINE_{—} | | | | | | | | |
| | b1_at | | | | | | | | |
| 15 | AFFX- | K00131 mouse b2 repeat sequence from clone mm61 | 2982.2 | P | 2633.7 | P | 2843.3 | P | 2720.3 |
| | MURINE_{—} | | | | | | | | |
| | B2_at | | | | | | | | |
| 15 | 92541_at | Cluster Incl X12973 Murine MLC1F/MLC3F gene for myosin alkali light chain (exon 1) (last skeletal muscle isoform)/ | 4135.8 | P | 3872.6 | P | 3679 | P | 3906.3 |
| | | cds = (125,238)/gb = X12973/gi = 53140/ug = Mm.1000/len = 250/STRA = for | | | | | | | |
| 15 | 92543_at | Cluster Incl AW123542 UI-M-BH2.1-aqb-f-01-0-UI.s1 Mus musculus cDNA, 3 end/clone = UI-M-BH2.1-aqb-f-01-0-UI/ | 222.2 | P | 188.5 | P | 245.5 | P | 239.5 |
| | | clone_end = 3/gb = AW123542/gi = 6099072/ug = Mm 100400/len = 373/STRA = for | | | | | | | |
| 15 | 92577_f_at | Cluster Incl c UI-M-BH1-alu-a-05-0-UI s1 Mus musculus cDNA, 3 end/clone = UI-M-BH1-alu-a-05-0-UI/clone_end = 3/ | 5562.9 | P | 4568.5 | P | 4983.4 | P | 5156.2 |
| | | gb = AW047116/gi = 5907645/ug = Mm 10474/len = 309/STRA = for | | | | | | | |
| 15 | 92624_r_at | Cluster Incl U09189: Mus musculus Ioricrin gene, complete cds/cds = (23,1468)/gb = U09189/gi = 520479/ug = Mm 1121/ | 3209.9 | P | 3106.2 | P | 3292.2 | P | 3417.1 |
| | | len = 1704/STRA = for | | | | | | | |
| 15 | 92628_at | Cluster Incl X75895 M musculus mRNA for ribosomal protein L36/cds = (20,337)/gb = X75895/gi = 440801/ug = Mm 11376/ | 5632.3 | P | 5610.1 | P | 5522 | P | 5614.4 |
| | | len = 387/STRA = for | | | | | | | |
| 15 | 92629_f_at | Cluster Incl D63707 Mouse mRNA for hepatoma derived growth factor (HDGF), complete cds/cds = (61,774)/gb = D63707/ | 2965.4 | P | 3507.6 | P | 3406.3 | P | 3470.2 |
| | | gi = 945418/ug = Mm 1141/len = 1563/STRA = for | | | | | | | |
| 15 | 92631_f_at | Cluster Incl M19380 Calmodulin 3/cds = (109,558)/gb = M19380/gi = 469419/ug = Mm 1147/len = 722/STRA = for | 739.5 | P | 926.7 | P | 696.2 | P | 573.2 |
| 15 | 92783_at | Cluster Incl X02891 Growth hormone/cds = (61,711)/gb = X02891/gi = 51067/ug = Mm.1240/len = 777/STRA = for | 597 | P | 1142.5 | P | 916.9 | P | 971.5 |
| 15 | 92880_at | Cluster Incl M38337 Milk fat globule-EGF factor 8 protein/cds = (45,1436)/gb = M38337/gi = 199142/ug = Mm 1451/len = 2077/ | 1854 | P | 1610.8 | P | 1521.6 | P | 1497.6 |
| | | STRA = for | | | | | | | |
| 15 | 92881_at | Cluster Incl U77943 Mus musculus myosin light chain 2 mRNA, complete cds/cds = (0,509)/gb = U77943/gi = 1675395/ | 1837 | P | 1907.5 | P | 2101.5 | P | 2213.4 |
| | | ug = Mm 14526/len = 580/STRA = for | | | | | | | |
| 15 | 93030_at | Cluster Incl AI844457: UI-M-AJ1-ahe-c-02-0-UI s1 Mus musculus cDNA, 3 end/clone = UI-M-AJ1-ahe-c-02-0-UI/ | 3885.7 | P | 3875.8 | P | 3735.7 | P | 3713.6 |
| | | clone_end = 3/gb = AI844457/gi = 5488363/ug = Mm 14833/len = 347/STRA = for | | | | | | | |
| 15 | 93038_f_at | Cluster Incl M69260: Lipocortin 1/cds = (14,1054)/gb = M69260/gi = 198844/ug = Mm.14860/len = 1340/STRA = for | 3858.8 | P | 3760.1 | P | 3831.3 | P | 4015.5 |
| 15 | 93076_at | Cluster Incl AW124171: UI-M-BH2 1-apr-h-07-0-UI s1 Mus musculus cDNA, 3 end/clone = UI-M-BH2 1-apr-h-07-0-UI/ | 2294.8 | P | 2252.2 | P | 2432.1 | P | 2495.9 |
| | | clone_end = 3/gb = AW124171/gi = 6099701/ug = Mm 1581/len = 173/STRA = for | | | | | | | |
| 15 | 93077_s_at | Cluster Incl D86232 Lymphocyte antigen 6 complex, locus C/cds = (144,452)/gb = D86232/gi = 1695689/ug = Mm 1583/ | 2174 | P | 874.9 | P | 1922.4 | P | 2429.7 |
| | | len = 2683/STRA = for | | | | | | | |
| 15 | 93101_s_at | Cluster Incl U96635 Neural precursor cell expressed, developmentally down-regulated gene 4/cds = (611,2944)/gb = U96635/ | 1530.1 | P | 1933.3 | P | 1994.9 | P | 1673.6 |
| | | gi = 2088622/ug = Mm 16553/len = 5581/STRA = for | | | | | | | |
| 15 | 93116_at | Cluster Incl J02626 Protein kinase, cAMP dependent, catalytic, beta/cds = (192,1247)/gb = J02826/gi = 200386/ | 702.1 | P | 894.7 | P | 467.7 | P | 1103.3 |
| | | ug = Mm 16766/len = 1272/STRA = for | | | | | | | |
| 15 | 93271_s_at | Cluster Incl AJ245401: Mus musculus Nesp gene for neuroendocrine secretory peptide 55/cds = (0,761)/gb = AJ245401/ | 4262.5 | P | 3546.6 | P | 3296.4 | P | 3950.4 |
| | | gi = 6010255/ug = Mm 17446/len = 762/STRA = for | | | | | | | |
| 15 | 93324_at | Cluster Incl M58566 Mouse TIS11 primary response gene, complete cds/cds = (67,1083)/gb = M58566/gi = 202064/ | 2378.7 | P | 1989.4 | P | 2507.1 | P | 2108.1 |
| | | ug = Mm 18571/len = 2659/STRA = for | | | | | | | |
| 15 | 93534_at | Cluster Incl X53929 Decorin/cds = (231,1295)/gb = X53929/gi = 53668/ug = Mm 1987/len = 1372/STRA = for | 4460 | P | 4213.5 | P | 4653.5 | P | 4421.3 |
| 15 | 93535_at | Cluster Incl AF041472: Mus musculus ataxin-2 (SCA2) mRNA, complete cds/cds = (26,3883)/gb = AF041472/gi = 3005019/ | 465.4 | P | 477 | P | 453.6 | P | 412.8 |
| | | ug = Mm 1989/len = 4225/STRA = for | | | | | | | |
| 15 | 93543_f_at | Cluster Incl J03952 Mouse, glutathione transferase GT8 7 mRNA, complete cds/cds = (12,668)/gb = J03952/gi = 193687/ | 1378.3 | P | 1110.1 | P | 1336.8 | P | 907.6 |
| | | ug = Mm 2011/len = 1018/STRA = for | | | | | | | |
| 15 | 93604_f_at | Cluster Incl AF061260 Mus musculus immunosuperfamily protein BI2 mRNA, complete cds/cds = (308,1195)/ | 1169 | P | 1428.1 | P | 1370.6 | P | 1481 |
| | | gb = AF061260/gi = 3779241/ug = Mm 20921/len = 1861/STRA = for | | | | | | | |
| 15 | 93730_at | Cluster Incl AF085809 Mus musculus synapsin lb (Synl) mRNA, complete cds/cds = (135,2147)/gb = AF085809/gi = 3599472/ | 3155.7 | P | 2162.4 | P | 2814.4 | P | 2958.5 |
| | | ug = Mm 20936/len = 3694/STRA = for | | | | | | | |
| 15 | 93740_at | Cluster Incl U33196 Mus musculus Y-box binding protein mYB-1a mRNA, complete cds/cds = (115,1083)/gb = U33196/ | 3325 | P | 3504.8 | P | 3201.7 | P | 3160 |
| | | gi = 988280/ug = Mm 21054/len = 1194/STRA = for | | | | | | | |
| 15 | 93744_at | Cluster Incl AI119347 ue98e05.y1 Mus musculus cDNA, 5 end/clone = IMAGE-1499168/clone_end = 5/gb = AI119347/ | 3112.2 | P | 2167.1 | P | 2548.8 | P | 2390.9 |
| | | gi = 3519671/ug = Mm 21075/len = 476/STRA = for | | | | | | | |
| 15 | 93748_at | Cluster Incl AW123901.UI-M-BH2 1-app-g-06-0-UI.s1 Mus musculus cDNA, 3 end/clone = UI-M-BH2 1-app-g-06-0-UI/ | 1531.4 | P | 1297.3 | P | 1488.2 | P | 1240.3 |
| | | clone_end = 3/gb = AW123901/gi = 6099431/ug = Mm 21094/len = 226/STRA = for | | | | | | | |
| 15 | 93762_at | Cluster Incl AI841493 UI-M-AH0-acy-d-06-0-UI s1 Mus musculus cDNA, 3 end/clone = UI-M-AH0-acy-d-06-0-UI/ | 1353.3 | P | 1408.2 | P | 1316.7 | P | 1400.3 |
| | | clone_end = 3/gb = AI841493/gi = 5475706/ug = Mm 21158/len = 367/STRA = for | | | | | | | |
| 15 | 93991_at | Cluster Incl X07295 Malate dehydrogenase, mitochondrial/cds = (0,1016)/gb = X07295/gi = 50679/ug = Mm.21743/len = 1017/ | 3814.3 | P | 3970.6 | P | 3518.5 | P | 3957.3 |
| | | STRA = for | | | | | | | |
| 15 | 94068_at | Cluster Incl AW048899 UI-M-BH1-amf-h-03-0-UI s1 Mus musculus cDNA, 3 end/clone = UI-M-BH1-amf-h-03-0-UI/ | 3080.4 | P | 2804.8 | P | 2841.8 | P | 2832.4 |
| | | clone_end = 3/gb = AW048899/gi = 5909428/ug = Mm 22103/len = 481/STRA = for | | | | | | | |
| 15 | 94247_at | Cluster Incl AA600542: vm75a03 r1 Mus musculus cDNA, 5 end/clone = IMAGE-1004044/clone_end = 5/gb = AA600542/ | 4401.7 | P | 4191.2 | P | 4163.7 | P | 4412.1 |
| | | gi = 2434167/ug = Mm 22365/len = 140/STRA = for | | | | | | | |
| 15 | 94305_at | Cluster Incl U03419: Mus musculus alpha-1 type I procollagen mRNA, partial cds/cds = (0,977)/gb = U03419/gi = 424103/ | 4348.8 | P | 3177.8 | P | 3092.4 | P | 3765.3 |
| | | ug = Mm.22621/len = 1205/STRA = for | | | | | | | |
| 15 | 94321_at | Cluster Incl V00830 Keratin complex 1, acidic, gene 10/cds = (27,1739)/gb = V00830/gi = 52786/ug = Mm.22662/len = 1944/ | 3481.2 | P | 3378.8 | P | 3279.8 | P | 3616.5 |
| | | STRA = for | | | | | | | |
| 15 | 94823_at | Cluster Incl AI835315 UI-M-AQ0-aah-a-06-0-UI s1 Mus musculus cDNA, 3 end/clone = UI-M-AQ0-aah-a-06-0-UI/ | 4983.7 | P | 4429.1 | P | 4618.9 | P | 4858.1 |
| | | clone_end = 3/gb = AI835315/gi = 5469528/ug = Mm 22723/len = 275/STRA = for | | | | | | | |
| 15 | 94839_at | Cluster Incl M96823: Mouse nucleobindin mRNA, complete cds/cds = (18,1385)/gb = M96823/gi = 200113/ug = Mm.2283/ | 656.7 | P | 821.8 | P | 686.2 | P | 483.2 |
| | | len = 2116/STRA = for | | | | | | | |
| 15 | 94845_at | Cluster Incl AW125736: UI-M-BH2.2-aqi-g-02-0-U1.s1 Mus musculus cDNA, 3 end/clone = UI-M-BH2.2-aqi-g-02-0-Ul/ | 951.9 | P | 808.1 | P | 756.4 | P | 1148.2 |
| | | clone_end = 3/gb = AW125736/gi = 6101231/ug = Mm.23014/len = 426/STRA = for | | | | | | | |
| 15 | 95435_at | Cluster Incl AI844009 UI-M-AL1-ahi-a-05-0-UI s1 Mus musculus cDNA, 3 end/clone = UI-M-AL1-ahi-a-05-0-UI/ | 263.2 | P | 261.2 | P | 271.3 | P | 322.7 |
| | | clone_end = 3/gb = AI844009/gi = 5487910/ug = Mm 24499/len = 408/STRA = for | | | | | | | |
| 15 | 95440_at | Cluster Incl AI846093 UI-M-AK1-aew-c-02-0-UI.s1 Mus musculus cDNA, 3 end/clone = UI-M-AK1-aew-c-02-0-UI/ | 184 | P | 215.5 | P | 202.1 | P | 162.1 |
| | | clone_end = 3/gb = AI846093/gi = 5489964/ug = Mm 24564/len = 284/STRA = for | | | | | | | |
| 15 | 95458_s_at | Cluster Incl AW121960 UI-M-BH2 3-aoi-h-09-0-UI s1 Mus musculus cDNA, 3 end/clone = UI-M-BH2 3-aoi-h-09-0-UI/ | 1728.7 | P | 1253.6 | P | 1107.6 | P | 1450.6 |
| | | clone_end = 3/gb = AW121960/gi = 6097311/ug = Mm 24703/len = 370/STRA = for | | | | | | | |
| 15 | 95490_at | Cluster Incl AW120891: UI-M-BH2 3-aob-b-02-0-UI s1 Mus musculus cDNA, 3 end/clone = UI-M-BH2.3-aob-b-02-0-UI/ | 1393.4 | P | 1686.9 | P | 1631.9 | P | 1435.8 |
| | | clone_end = 3/gb = AW120891/gi = 6096224/ug = Mm 25045/len = 178/STRA = for | | | | | | | |
| 15 | 96018_r_at | Cluster Incl AI853561 UI-M-BH0-ajd-h-09-0-UI s1 Mus musculus cDNA, 3 end/clone = UI-M-BH0-ajd-h-09-0-UI/ | 357.2 | P | 306.1 | P | 347.6 | P | 393.1 |
| | | clone_end = 3/gb = AI853561/gi = 5497467/ug = Mm 25634/len = 480/STRA = for | | | | | | | |
| 15 | 96066_s_at | Cluster Incl X97047: Pyruvate kinase 3/cds = (6,1601)/gb = X97047/gi = 1405932/ug = Mm.2635/len = 2134/STRA = for | 2752 | P | 3222.9 | P | 3309.2 | P | 3510.8 |
| 15 | 96072_at | Cluster Incl M17516: Mouse lactate dehydrogenase A-4 (LDH-A) mRNA, complete cds/cds = (0,395)/gb = M17516/ | 4057.9 | P | 3888.6 | P | 3672.6 | P | 4065.4 |
| | | gi = 198776/ug = Mm.26504/len = 881/STRA = for | | | | | | | |
| 15 | 96699_at | Cluster Incl X53476 High mobility group protein 14/cds = (33,323)/gb = X53476/gi = 51334/ug = Mm.2756/len = 1070/ | 5251 | P | 5170.4 | P | 5240.8 | P | 5230.3 |
| | | STRA = for | | | | | | | |
| 15 | 97229_at | Cluster Incl AW061042: UI-M-BH1-akk-b-09-0-Ul s2 Mus musculus cDNA, 3 end/clone = UI-M-BH1-akk-b-09-0-UI/ | 1941 | P | 1592.5 | P | 1714.5 | P | 1690.7 |
| | | clone_end = 3/gb = AW061042/gi = 6008793/ug = Mm 27796/len = 502/STRA = for | | | | | | | |
| 15 | 97808_at | Cluster Incl AI844532 UI-M-AJ1-ahf-d-05-0-UI.s2 Mus musculus cDNA, 3 end/clone = UI-M-AJ1-ahf-d-05-0-UI/ | 1213.8 | P | 800.8 | P | 837.8 | P | 1313.2 |
| | | clone_end = 3/gb = AI844532/gi = 5488438/ug = Mm 28150/len = 521/STRA = for | | | | | | | |
| 15 | 97825_at | Cluster Incl AI854029 UI-M-BH0-ajm-c-04-0-UI s1 Mus musculus cDNA, 3 end/clone = UI-M-BH0-ajm-c-04-0-UI/ | 5072.4 | P | 4725.5 | P | 4940.6 | P | 4962.8 |
| | | clone_end = 3/gb = AI854029/gi = 5497935/ug = Mm 28209/len = 528/STRA = for | | | | | | | |
| 15 | 98922_at | Cluster Incl L34260 Integral membrane protein 1/cds = (111,2228)/gb = L34260/gi = 508542/ug = Mm 2863/len = 3094/ | 165.2 | P | 128.6 | P | 122.7 | P | 121.8 |
| | | STRA = for | | | | | | | |
| 15 | 98927_at | Cluster Incl AI851048: UI-M-BH0-ajv-f-08-0-UI s1 Mus musculus cDNA, 3 end/clone = UI-M-BH0-ajv-f-08-0-UI/ | 1320.5 | P | 1260.9 | P | 1353.5 | P | 1476.7 |
| | | clone_end = 3/gb = AI851048/gi = 5494954/ug = Mm 28650/len = 129/STRA = for | | | | | | | |
| 15 | 94464_at | Cluster Incl AF029347 Mus musculus chloride channel protein 3 (CLCN3) mRNA, complete cds/cds = (0,2456)/ | 242 | P | 222.9 | P | 189.7 | P | 199.9 |
| | | gb = AF029347/gi = 2599549/ug = Mm.28842/len = 2457/STRA = for | | | | | | | |
| 15 | 94481_at | Cluster Incl AI851321 UI-M-BH0-akh-a-06-0-UI s1 Mus musculus cDNA, 3 end/clone = UI-M-BH0-akh-a-06-0-UI/ | 414.2 | P | 446.7 | P | 415.1 | P | 531.2 |
| | | clone_end = 3/gb = AI851321/gi = 5495227/ug = Mm 28877/len = 420/STRA = for | | | | | | | |
| 15 | 94536_s_at | Cluster Incl AI843417: UI-M-AQ1-aec-a-10-0-UI s1 Mus musculus cDNA, 3 end/clone = UI-M-AQ1-aec-a-10-0-UI/ | 3227 | P | 3146.7 | P | 3112.7 | P | 3211.9 |
| | | clone_end = 3/gb = AI843417/gi = 5477630/ug = Mm 29057/len = 345/STRA = for | | | | | | | |
| 15 | 95046_s_at | Cluster Incl M98036 Mus musculus guanine nucleotide exchange factor delta subunit (JGR1A) mRNA, complete cds/ | 298.9 | P | 559 | P | 464.4 | P | 538.6 |
| | | cds = (69,1643)/gb = M98036/gi = 529429/ug = Mm 29128/len = 1677/STRA = for | | | | | | | |
| 15 | 95064_at | Cluster Incl AI849271 UI-M-AJ1-agz-g-06-0-UI s1 Mus musculus cDNA, 3 end/clone = UI-M-AJ1-agz-g-06-0-UI/ | 4019.9 | P | 3984.5 | P | 3909 | P | 3977.1 |
| | | clone_end = 3/gb = AI849271/gi = 5493177/ug = Mm 29165/len = 405/STRA = rev | | | | | | | |
| 15 | 95072_at | Cluster Incl AW121892 UI-M-BH2.3-aoi-e-12-0-UI s1 Mus musculus cDNA, 3 end/clone = UI-M-BH2 3-aoi-e-12-0-UI/ | 2973.9 | P | 3273.9 | P | 2852.3 | P | 3209.3 |
| | | clone_end = 3/gb = AW121892/gi = 6097266/ug = Mm 29196/len = 438/STRA = for | | | | | | | |
| 15 | 95124_i_at | Cluster Incl AW122337: UI-M-BH2.2-aos-g-04-0-UI s1 Mus musculus cDNA, 3 end/clone = UI-M-BH2 2-aos-g-04-0-UI/ | 654.9 | P | 702.1 | P | 725.8 | P | 1129.7 |
| | | clone_end = 3/gb = AW122337/gi = 6097822/ug = Mm 29405/len = 403/STRA = for | | | | | | | |
| 15 | 95125_f_at | Cluster Incl AW122337: UI-M-BH2 2-aos-g-04-0-UI s1 Mus musculus cDNA, 3 end/clone = UI-M-BH2 2-aos-g-04-0-UI/ | 1880.6 | P | 1829.2 | P | 1663 | P | 1850.9 |
| | | clone_end = 3/gb = AW122337/gi = 6097822/ug = Mm 29405/len = 403/STRA = for | | | | | | | |
| 15 | 95132_r_at | Cluster Incl AI852592 UI-M-BH0-aiu-b-11-0-UI s1 Mus musculus cDNA, 3 end/clone = UI-M-BH0-aiu-b-11-0-UI/ | 1549.9 | P | 1544.5 | P | 1588.5 | P | 1671.5 |
| | | clone_end = 3/gb = AI852592/gi = 5496498/ug = Mm 29415/len = 392/STRA = rev | | | | | | | |
| 15 | 95661_at | Cluster Incl L08115: CD9 antigen/cds = (54,734)/gb = L08115/gi = 388911/ug = Mm 2956/len = 1130/STRA = for | 3117.9 | P | 3363.1 | P | 2951.5 | P | 3295.5 |
| 15 | 95698_at | Cluster Incl AI851810: UI-M-BH0-ais-c-02-0-UI s1 Mus musculus cDNA, 3 end/clone = UI-M-BH0-ais-c-02-0-UI/ | 1386.2 | P | 2275.4 | P | 1963.5 | P | 1872.6 |
| | | clone_end = 3/gb = AI851810/gi = 5495716/ug = Mm 29683/len = 276/STRA = for | | | | | | | |
| 15 | 95705_s_at | Cluster Incl J04181: Mouse A-X actin mRNA complete cds/cds = (62,1189)/gb = J04181/gi = 191660/ug = Mm 297/len = 1857/ | 3594.3 | P | 4528.2 | P | 3725.9 | P | 3576.6 |
| | | STRA = for | | | | | | | |
| 15 | 95706_at | Cluster Incl X16834 Mouse mRNA for Mac-2 antigen/cds = (43,837)/gb = X16834/gi = 52986/ug = Mm 2970/len = 955/ | 2500.4 | P | 2540.2 | P | 2590.3 | P | 2609.3 |
| | | STRA = for | | | | | | | |
| 15 | 95713_at | Cluster Incl D67067 Mouse mRNA for thymic epithelial cell surface antigen, complete cds/cds = (12,1559)/gb = D67067/ | 1771.9 | P | 1752.2 | P | 1661.2 | P | 1737.2 |
| | | gi = 2059325/ug = Mm 29710/len = 2446/STRA = for | | | | | | | |
| 15 | 95723_r_at | Cluster Incl AI841464: UI-M-AH0-acy-a-04-0-UI.s1 Mus musculus cDNA, 3 end/clone = UI-M-AH0-acy-a-04-0-UI/ | 2813 | P | 2748 | P | 2587.2 | P | 2627.8 |
| | | clone_end = 3/gb = AI841464/gi = 5475677/ug = Mm.29731/len = 413/STRA = for | | | | | | | |
| 15 | 95740_at | Cluster Incl AA727291: vu95a10 r1 Mus musculus cDNA, 5 end/clone = IMAGE-1209882/clone_end = 5/gb = AA727291/ | 4096.2 | P | 3873.5 | P | 3732.5 | P | 3974.6 |
| | | gi = 2744998/ug = Mm.29753/len = 279/STRA = for | | | | | | | |
| 15 | 95755_at | Cluster Incl D14485: Mouse mRNA for dbpA murine homologue, complete cds/cds = (183,1109)/gb = D14485/gi = 1160330/ | 2773.3 | P | 2631.6 | P | 2845.9 | P | 2863.5 |
| | | ug = Mm 29794/len = 1647/STRA = for | | | | | | | |
| 15 | 96290_f_at | Cluster Incl U93863: Mus musculus ribosomal protein L21 mRNA, complete cds/cds = (36,518)/gb = U93863/gi = 1938403/ | 5671.4 | P | 5383.6 | P | 5504.8 | P | 5523.4 |
| | | ug = Mm 29896/len = 612/STRA = for | | | | | | | |
| 15 | 96298_f_at | Cluster Incl AF020185: Mus musculus protein inhibitor of nitric oxide synthase (PIN) mRNA, complete cds/cds = (99,388)/ | 5436.1 | P | 4901.1 | P | 5348.9 | P | 5470.5 |
| | | gb = AF020185/gi = 4103058/ug = Mm.29908/len = 531/STRA = for | | | | | | | |
| 15 | 96300_f_at | Cluster Incl AI8542318 UI-M-BH0-ajz-g-10-0-UI s1 Mus musculus cDNA, 3 end/clone = UI-M-BH0-ajz-g-10-0-UI/ | 3876.9 | P | 3159.1 | P | 3495.2 | P | 3409.6 |
| | | clone_end = 3/gb = AI854238/gi = 5498144/ug = Mm.29911/len = 349/STRA = for | | | | | | | |
| 15 | 96307_s_at | Cluster Incl AW121947 UI-M-BH2 3-aoj-f-05-0-UI s1 Mus musculus cDNA, 3 end/clone = UI-M-BH2 3-aoj-f-05-0-UI/ | 3169.5 | P | 2365.2 | P | 2597.6 | P | 2770.8 |
| | | clone_end = 3/gb = AW121947/gi = 6097393/ug = Mm.29919/len = 347/STRA = for | | | | | | | |
| 15 | 96327_at | Cluster Incl AI852535: UI-M-BH0-aiw-e-05-0-UI.s1 Mus musculus cDNA, 3 end/clone = UI-M-BH0-aiw-e-05-0-UI/ | 658.3 | P | 574 | P | 351.7 | P | 531.5 |
| | | clone_end = 3/gb = AI852535/gi = 5496441/ug = Mm 29962/len = 357/STRA = for | | | | | | | |
| 15 | 96358_at | Cluster Incl AI837403: UI-M-AP0-abm-e-02-0-UI.s1 Mus musculus cDNA, 3 end/clone = UI-M-AP0-abm-e-02-0-UI/ | 2850 | P | 1491.9 | P | 2572.4 | P | 2381 |
| | | clone_end = 3/gb = AI837403/gi = 5471616/ug = Mm.30011/len = 404/STRA = for | | | | | | | |
| 15 | 96859_at | Cluster Incl AB026621: Mus musculus mRNA for RIE2. complete cds/cds = (481,2895)/gb = AB026621/gi = 5931597/ | 2065.3 | P | 1758.1 | P | 2004.7 | P | 1810.2 |
| | | ug = Mm 30051/len = 3110/STRA = for | | | | | | | |
| 15 | 96872_at | Cluster Incl AI835846: UI-M-AI0-aat-b-06-0-UI s1 Mus musculus cDNA, 3 end/clone = UI-M-AI0-aat-b-06-0-UI/clone_end = 3/ | 1344.6 | P | 1305 | P | 1261.1 | P | 1406 |
| | | gb = AI835846/gi = 5470059/ug = Mm.30068/len = 182/STRA = for | | | | | | | |
| 15 | 96942_at | Cluster Incl AI844011: UI-M-AL1-ahi-b-01-0-UI s1 Mus musculus cDNA, 3 end/clone = UI-M-AL1-ahi-b-01-0-UI/ | 2797.1 | P | 2863.2 | P | 2944.5 | P | 2927.9 |
| | | clone_end = 3/gb = AI844011/gi = 5487917/ug = Mm 30194/len = 338/STRA = for | | | | | | | |
| 15 | 96949_at | Cluster Incl AI853542 UI-M-BH0-ajd-g-01-0-UI s1 Mus musculus cDNA, 3 end/clone = UI-M-BH0-ajd-g-01-0-UI/ | 795 | P | 736.4 | P | 740.1 | P | 789.9 |
| | | clone_end = 3/gb = AI853542/gi = 5497448/ug = Mm 30205/len = 110/STRA = for | | | | | | | |
| 15 | 97460_at | Cluster Incl AW122823: UI-M-BH2 1-aoz-c-09-0-UI.s1 Mus musculus cDNA, 3 end/clone = UI-M-BH2 1-aoz-c-09-0-UI/ | 1516.8 | P | 1689 | P | 1758.9 | P | 1642.6 |
| | | clone_end = 3/gb = AW122823/gi = 6098353/ug = Mm 30272/len = 219/STRA = for | | | | | | | |
| 15 | 97484_at | Cluster Incl AI852704: UI-M-BH0-aji-e-07-0-UI s1 Mus musculus cDNA, 3 end/clone = UI-M-BH0-aji-e-07-0-UI/clone_end = 3/ | 827.8 | P | 864.5 | P | 636.2 | P | 709.8 |
| | | gb = AI852704/gi = 5496610/ug = Mm.30826/len = 427/STRA = for | | | | | | | |
| 15 | 97544_at | Cluster Incl D83037 Mouse mRNA for 14-3-3 zeta, complete cds/cds = (87,824)/gb = D83037/gi = 1181605/ug = Mm 3360/ | 2250.5 | P | 2068.7 | P | 2183.1 | P | 2147.4 |
| | | len = 1935/STRA = for | | | | | | | |
| 15 | 97553_at | Cluster Incl AI841884 UI-M-AO0-acd-c-10-0-UI.s1 Mus musculus cDNA, 3 end/clone = UI-M-AO0-acd-c-10-0-UI/ | 394.6 | P | 413.8 | P | 476.9 | P | 546.4 |
| | | clone_end = 3/gb = AI841884/gi = 5476097/ug = Mm 33808/len = 370/STRA = for | | | | | | | |
| 15 | 98059_s_at | Cluster Incl D49733: Lamin A/cds = (0,1388)/gb = D49733/gi = 1838920/ug = Mm.3438/len = 1480/STRA = for | 4620 | P | 4755 | P | 4703.6 | P | 4769.9 |
| 15 | 98078_at | Cluster Incl AI157911 ue57h08 r1 Mus musculus cDNA, 5 end/clone = IMAGE-1495263/clone_end = 5/gb = AI157911/ | 251.2 | P | 264.9 | P | 278.3 | P | 341.9 |
| | | gi = 3686380/ug = Mm 34556/len = 439/STRA = for | | | | | | | |
| 15 | 98094_f_at | Cluster Incl AI843627 UI-M-AO1-aen-f-03-0-UI.s1 Mus musculus cDNA, 3 end/clone = UI-M-AO1-aen-f-03-0-UI/ | 594.6 | P | 589.3 | P | 571.8 | P | 589.1 |
| | | clone_end = 3/gb = AI843627/gi = 5477840/ug = Mm.34641/len = 425/STRA = for | | | | | | | |
| 15 | 98119_at | Cluster Incl K02928 Ribosomal protein L30/cds = (70,417)/gb = K02928/gi = 435126/ug = Mm 3487/len = 468/STRA = for | 5110 | P | 4614.9 | P | 4395.9 | P | 4761.6 |
| 15 | 98152_at | Cluster Incl AW122407 UI-M-BH2 2-aow-e-09-0-UI.s1 Mus musculus cDNA, 3 end/clone = UI-M-BH2.2-aow-e-09-0-UI/ | 1483.5 | P | 1277.7 | P | 1316.3 | P | 1353.4 |
| | | clone_end = 3/gb = AW122407/gi = 6097909/ug = Mm 35738/len = 471/STRA = for | | | | | | | |
| 15 | 98154_at | Cluster Incl AW050133 UI-M-BH1-ano-d-11-0-UI s1 Mus musculus cDNA, 3 end/clone = UI-M-BH1-ano-d-11-0-UI/ | 296.2 | P | 302.7 | P | 268.1 | P | 369.7 |
| | | clone_end = 3/gb = AW050133/gi = 5910662/ug = Mm 35779/len = 521/STRA = for | | | | | | | |
| 15 | 98557_f_at | Cluster Incl U65636 Mus musculus beta proteasome subunit (Lmp3) mRNA, complete cds/cds = (5,799)/gb = U65636/ | 2382.2 | P | 2623.6 | P | 2138.9 | P | 2468.6 |
| | | gi = 1762778/ug = Mm 368/len = 892/STRA = for | | | | | | | |
| 15 | 98564_f_at | Cluster Incl U67770 Mus musculus ribosomal protein S26 (RPS26) mRNA, complete cds/cds(5,352)/gb = U67770/ | 3730 | P | 2812.4 | P | 2823.1 | P | 3061.5 |
| | | gi = 1527175/ug = Mm 372/len = 426/STRA = for | | | | | | | |
| 15 | 98571_s_at | Cluster Incl U48363 Nascent polypeptide-associated complex alpha polypeptide/cds = (51,698)/gb = U48363/gi = 1686688/ | 4154.9 | P | 4153.3 | P | 4103.6 | P | 4114.5 |
| | | ug = Mm 3746/len = 829/STRA = for | | | | | | | |
| 15 | 99093_at | Cluster Incl AI852864: UI-M-BH0-ajk-e-04-0-UI s1 Mus musculus cDNA, 3 end/clone = UI-M-BH0-ajk-e-04-0-UI/ | 5263.7 | P | 4683.7 | P | 4939.9 | P | 5077.7 |
| | | clone_end = 3/gb = AI852864/gi = 5496770/ug = Mm 39130/len = 308/STRA = for | | | | | | | |
| 15 | 99096_at | Cluster Incl U46690 Mus musculus ATP-dependent RNA helicase mRNA, partial cds/cds = (0,1441)/gb = U46690/ | 260.6 | P | 273.5 | P | 280.7 | P | 239.1 |
| | | gi = 1262844/ug = Mm 3935/len = 1674/STRA = for | | | | | | | |
| 15 | 99120_f_at | Cluster Incl R75450 MDB0625 Mus musculus cDNA, 3 end/clone_end = 3/gb = R75450/gi = 849694/ug = Mm.4024/ | 2002.4 | P | 2304.4 | P | 2008.9 | P | 2117.7 |
| | | len = 329/STRA = for | | | | | | | |
| 15 | 99126_at | Cluster Incl L04961 Inactive X specific transcripts/cds = UNKNOWN/gb = L04961/gi = 202420/ug = Mm 4095/len = 14739/ | 2979.1 | P | 2259.7 | P | 2223.9 | P | 3613.8 |
| | | STRA = for | | | | | | | |
| 15 | 99135_at | Cluster Incl U43076 Mus musculus cdc37 homolog mRNA, complele cds/cds = (87,1226)/gb = U43078/gi = 1375488/ | 952.7 | P | 1015.3 | P | 1040.8 | P | 913.8 |
| | | ug = Mm 4117/len = 1607/STRA = for | | | | | | | |
| 15 | 99586_at | Cluster Incl U10098 Cystalin 3/cds = (0,422)/gb = U10098/gi = 497414/ug = Mm 4263/len = 423/STRA = for | 4194.9 | P | 3853.4 | P | 4316.7 | P | 3955 |
| 15 | 99590_at | Cluster Incl D25213 Mouse rpS17 mRNA for ribosomal protein S17, complete cds/cds = (11,418)/gb = D25213/gi = 893394/ | 3630.3 | P | 3281.5 | P | 3431.8 | P | 3540.6 |
| | | ug = Mm 42767/len = 453/STRA = for | | | | | | | |
| 15 | 99592_f_at | Cluster Incl AB030505 Mus musculus mRNA for UBE-1c1, UBE-1c2, UBE-1c3, complete cds/cds = UNKNOWN/ | 1095.4 | P | 1110.8 | P | 787 | P | 935.6 |
| | | gb = AB030505/gi = 5668736/ug = Mm.42795/len = 4190/STRA = for | | | | | | | |
| 15 | 99640_at | Cluster Incl AW045481: UI-M-BH1-ahr-c-09-0-UI s1 Mus musculus cDNA, 3 end/clone = UI-M-BH1-ai.r-c-09-0-UI/ | 802.1 | P | 696 | P | 705.7 | P | 643.5 |
| | | clone_end = 3/gb = AW045481/gi = 5905975/ug = Mm 43580/len = 398/STRA = for | | | | | | | |
| 15 | 99660_f_at | Cluster Incl X52940: Cytochrome C oxidase subunit VIIc/cds = (86,277)/gb = X52940/gi = 50524/ug = Mm 43786/len = 403/ | 4163.6 | P | 3563.8 | P | 4013 | P | 3925 |
| | | STRA = for | | | | | | | |
| 15 | 99661_r_at | Cluster Incl X52940: Cytochrome C oxidase subunit VIIc/cds = (86,277)/gb = X52940/gi = 50524/ug = Mm.43786/len = 403/ | 2133.8 | P | 1767.8 | P | 1890.4 | P | 2051.9 |
| | | STRA = for | | | | | | | |
| 15 | 99669_at | Cluster Incl X15986: Mouse 3 mRNA for beta-galactoside specific lectin (14 kDa)/cds = (43,450)/gb = X15986/gi = 50162/ | 5173.1 | P | 4991.9 | P | 4863.2 | P | 4997.1 |
| | | ug = Mm 43831/len = 495/STRA = for | | | | | | | |
| 15 | 100043_f_at | Cluster Incl AI173973: ui43c05 x1 Mus musculus cDNA, 3 end/clone = IMAGE-1885160/clone_end = 3/gb = AI173973/ | 3274.6 | P | 3255.7 | P | 3060.3 | P | 2883.1 |
| | | gi = 3720115/ug = Mm 44242/len = 459/STRA = for | | | | | | | |
| 15 | 100064_f_at | Cluster Incl M63801 Mus musculus connexin 43 (alpha-1 gap junction) mRNA, complete cds/cds = (220,1388)/gb = M63801/ | 4672.4 | P | 4384.4 | P | 4435.2 | P | 4372.8 |
| | | gi = 191773/ug = Mm 4504/len = 1408/STRA = for | | | | | | | |
| 15 | 100569_at | Cluster Incl M14044 Mouse calpactin I heavy chain (p36) mRNA, complete cds/cds = (7,1026)/gb = M14044/gi = 192353/ | 4049.6 | P | 3557.8 | P | 3569.1 | P | 3666.7 |
| | | ug = Mm 584/len = 1307/STRA = for | | | | | | | |
| 15 | 100618_f_at | Cluster Incl AA062013 mj83h01 r1 Mus musculus cDNA, 5 end/clone = IMAGE-482737/clone_end = 5/gb = AA062013/ | 1204.1 | P | 1230.8 | P | 1165.9 | P | 1573.8 |
| | | gi = 1555829/ug = Mm.658/len = 529/STRA = for | | | | | | | |
| 15 | 101093_at | Cluster Incl M15832: Procollagen, type IV, alpha 1/cds = (0,689)/gb = M15832/gi = 192282/ug = Mm 738/len = 690/STRA = for | 3112 | P | 3855.3 | P | 3411.7 | P | 3681.8 |
| 15 | 101113_at | Cluster Incl AI846668 UI-M-AN1-afi-c-10-0-UI s1 Mus musculus cDNA, 3 end/clone = UI-M-AN1-afi-c-10-0-UI/clone_end = 3/ | 1568.3 | P | 1469.2 | P | 1588.4 | P | 1668.7 |
| | | gb = AI846668/gi = 5490574/ug = Mm.757/len = 262/STRA = for | | | | | | | |
| 15 | 101542_f_at | Cluster Incl L25126 D-E-A-D (aspartate-glutamahe-alanine-aspartate) box polypeptide 3/cds = (23,2011)/gb = L25126/ | 2094.7 | P | 2202.4 | P | 2545.5 | P | 2987.9 |
| | | gi = 407995/ug = Mm 88188/len = 2187/STRA = for | | | | | | | |
| 15 | 101543_f_at | Cluster Incl M13441: Mouse alpha-tubulin isotype M-alpha-6 mRNA, complete cds/cds = (43,1392)/gb = M13441/gi = 202216/ | 2841.8 | P | 2187.6 | P | 2600.6 | P | 2540.9 |
| | | ug = Mm.88212/len = 1495/STRA = for | | | | | | | |
| 15 | 101573_f_at | Cluster Incl AB020237: Mus musculus gene for ribosomal protein L27A, complete cds/cds = (0,446)/gb = AB020237/ | 4415.7 | P | 4346.3 | P | 4385.7 | P | 4413.8 |
| | | gi = 4760603/ug = Mm 89853/len = 447/STRA = for | | | | | | | |
| 15 | 101577_at | Cluster Incl Z54209 M. musculus rpS6 gene/cds = (41,790)/gb = Z54209/gi = 1177548/ug = Mm.89902/len = 831/STRA = for | 4202.9 | P | 3513.2 | P | 3829.4 | P | 3987.4 |
| 15 | 101578_f_at | Cluster Incl M12481 Actin, beta, cytoplasmic/cds = (0,1049)/gb = M12481/gi = 191581/ug = Mm.89919/len = 1109/STRA = for | 3643.7 | P | 4714.2 | P | 3843.1 | P | 4185.3 |
| 15 | 102000_f_at | Cluster Incl AI842835 UI-M-AN1-afb-a-07-0-UI s1 Mus musculus cDNA, 3 end/clone = UI-M-AN1-afb-a-07-0-UI/ | 1028.3 | P | 999.3 | P | 865.3 | P | 1032.8 |
| | | clone_end = 3/gb = AI842835/gi = 5477048/ug = Mm 988/len = 287/STRA = for | | | | | | | |
| 15 | 102028_at | Cluster Incl AF053959 Mus musculus putative ras effector Nore1 mRNA, complete cds/cds = (30,1271)/gb = AF053959/ | 492.2 | P | 474 | P | 583 | P | 532.6 |
| | | gi = 2997697/ug = Mm.10133/len = 3018/STRA = for | | | | | | | |
| 15 | 102364_at | Cluster Incl J04509 Jun proto-oncogene related gene d1/cds = (121,1146)/gb = J04509/gi = 198486/ug = Mm.1175/len = 1147/ | 3490 | P | 2989 | P | 2497.9 | P | 3253.9 |
| | | STRA = for | | | | | | | |
| 15 | 102414_i_at | Cluster Incl U28423 Mus musculus protein kinase inhibitor p58 mRNA, complete cds/cds = (100,1614)/gb = U28423/ | 342.8 | P | 279.3 | P | 203.7 | P | 373.6 |
| | | gi = 1353271/ug = Mm.12616/len = 1615/STRA = for | | | | | | | |
| 15 | 102856_at | Cluster Incl AF047389 SRY-box containing gene 10/cds = (53,931)/gb = AF047389/gi = 3264587/ug = Mm 1493/len = 1463/ | 481.8 | P | 438.3 | P | 453 | P | 564.1 |
| | | STRA = for | | | | | | | |
| 15 | 102986_at | Cluster Incl M18779 Myogenic differentiation 1/cds = (159,1115)/gb = M18779/gi = 199994/ug = Mm 1526/len = 1785/ | 299.2 | P | 255.4 | P | 256.6 | P | 325.6 |
| | | STRA = for | | | | | | | |
| 15 | 103079_at | Cluster Incl AA982208: vw61e08 s1 Mus musculus cDNA, 3 end/clone = IMAGE-1248326/clone_end = 3/gb = AA982208/ | 903.9 | P | 951.4 | P | 893.6 | P | 752.1 |
| | | gi = 3160867/ug = Mm 17166/len = 415/STRA = for | | | | | | | |
| 15 | 103082_at | Cluster Incl AI847507: UI-M-AM1-afv-a-08-0-UI s1 Mus musculus cDNA, 3 end/clone = UI-M-AM1-afv-a-08-0-UI/ | 671.9 | P | 597.1 | P | 467.6 | P | 527.1 |
| | | clone_end = 3/gb = AI847507/gi = 5491413/ug = Mm 17173/len = 313/STRA = for | | | | | | | |
| 15 | 103388_at | Cluster Incl AW047050: UI-M-BH1-alr-c-07-0-UI.s1 Mus musculus cDNA, 3 end/clone = UI-M-BH1-alr-c-07-0-UI/ | 1522.9 | P | 1181.6 | P | 1238.5 | P | 1409.5 |
| | | clone_end = 3/gb = AW047050/gi = 5907579/ug = Mm 1865/len = 318/STRA = rev | | | | | | | |
| 15 | 103611_at | Cluster Incl AB012693 Mus musculus mRNA for CD47, complete cds/cds = (72,1046)/gb = AB012693/gi = 3036964/ | 931.6 | P | 1117.8 | P | 1226.1 | P | 1072.2 |
| | | ug = Mm 20183/len = 1250/STRA = rev | | | | | | | |
| 15 | 103846_at | Cluster Incl AF038582 Mus musculus galectin-7 mRNA, complete cds/cds = (20,430)/gb = AF038562/gi = 2708653/ | 3310.4 | P | 2841.4 | P | 3192 | P | 3137.9 |
| | | ug = Mm 20973/len = 588/STRA = for | | | | | | | |
| 15 | 103862_r_at | Cluster Incl AA388099: vc88g07 r1 Mus musculus cDNA, 5 end/clone = IMAGE-790140/clone_end = 5/gb = AA388099/ | 1462.5 | P | 1431.2 | P | 1225 | P | 1414 |
| | | gi = 2041044/ug = Mm 21103/len = 564/STRA = for | | | | | | | |
| 15 | 103878_at | Cluster Incl AF103809 Mus musculus Ap-3 complex beta3A subunit (Ap3b1) mRNA, complete cds/cds = (63,3380)/ | 771.7 | P | 707.7 | P | 790.8 | P | 695.4 |
| | | gb = AF103809/gi = 3885987/ug = Mm 21185/len = 3938/STRA = for | | | | | | | |
| 15 | 103894_at | Cluster Incl AW060871: UI-M-BH1-amx-f-12-0-UI s1 Mus musculus cDNA, 3 end/clone = UI-M-BH1-amx-f-12-0-UI/ | 2394.1 | P | 2711.3 | P | 2531.8 | P | 2436.1 |
| | | clone_end = 3/gb = AW060871/gi = 6008622/ug = Mm 21297/len = 463/STRA = for | | | | | | | |
| 15 | 104106_at | Cluster Incl AI837830: UI-M-AP0-abk-a-07-0-UI s1 Mus musculus cDNA, 3 end/clone = UI-M-AP0-abk-a-07-0-UI/ | 249.9 | P | 172.6 | P | 201.1 | P | 321.8 |
| | | clone_end = 3/gb = AI837830/gi = 5472043/ug = Mm 21897/len = 259/STRA = for | | | | | | | |
| 15 | 104311_at | Cluster Incl AW125241: UI-M-BH2 1-aps-e-11-0-UI s1 Mus musculus cDNA, 3 end/clone = UI-M-BH2 1-aps-e-11-0-UI/ | 157.3 | P | 141.9 | P | −48.7 | P | 358.2 |
| | | clone_end = 3/gb = AW125241/gi = 6100771/ug = Mm 22397/len = 320/STRA = for | | | | | | | |
| 15 | 104313_at | Cluster Incl AI842432 UI-M-AM1-afy-b-10-0-UI s1 Mus musculus cDNA, 3 end/clone = UI-M-AM1-afy-b-10-0-UI/ | 1003.5 | P | 1308 | P | 1426 | P | 1109.6 |
| | | clone_end = 3/gb = AI842432/gi = 5476645/ug = Mm 22403/len = 330/STRA = for | | | | | | | |
| 15 | 104341_at | Cluster Incl AW121406 UI-M-BH2 2-aom-h-09-0-UI s1 Mus musculus cDNA, 3 end/clone = UI-M-BH2 2-aom-h-09-0-UI/ | 1109.9 | P | 1059.7 | P | 1356.6 | P | 1176.7 |
| | | clone_end = 3/gb = AW121406/gi = 6096739/ug = Mm 22525/len = 498/STRA = for | | | | | | | |
| 15 | 104362_at | Cluster Incl AI853532 UI-M-BHO-ajd-f-02-0-UI s1 Mus musculus cDNA, 3 end/clone = UI-M-BH0-ajd-f-02-0-UI/ | 339.8 | P | 447.5 | P | 403.5 | P | 389.3 |
| | | clone_end = 3/gb = AI853532/gi = 5497438/ug = Mm.22599/len = 353/STRA = for | | | | | | | |
| 15 | 104391_s_at | Cluster Incl AI850563 UI-M-BG1-aij-e-12-0-UI s1 Mus musculus cDNA, 3 end/clone = UI-M-BG1-aij-e-12-0-UI/ | 474.6 | P | 410.7 | P | 444.5 | P | 443.2 |
| | | clone_end = 3/gb = AI850563/gi = 5494469/ug = Mm.22727/len = 271/STRA = for | | | | | | | |
| 15 | 104524_at | Cluster Incl AI842825: UI-M-AP1-agi-h-07-0-UI s1 Mus musculus cDNA, 3 end/clone = UI-M-AP1-agi-h-07-0-UI/ | 3890.3 | P | 3448 | P | 3565.8 | P | 3769.7 |
| | | clone_end = 3/gb = AI842825/gi = 5477038/ug = Mm 22986/len = 341/STRA = for | | | | | | | |
| 15 | 104559_at | Cluster Incl AI848162 UI-M-AH1-agq-c-02-0-UI s1 Mus musculus cDNA, 3 end/clone = UI-M-AH1-asg-c-02-0-UI/ | 1179.8 | P | 1225.7 | P | 1422.4 | P | 1293.1 |
| | | clone_end = 3/gb = AI848162/gi = 5492068/ug = Mm 23826/len = 492/STRA = for | | | | | | | |
| 15 | 104576_at | Cluster Incl AW212397: uo86e12.x1 Mus musculus cDNA, 3 end/clone = IMAGE-2649454/clone_end = 3/gb = AW212397/ | 1273.9 | P | 1281.8 | P | 1249 | P | 1065.6 |
| | | gi = 6518452/ug = Mm 23941/len = 165/STRA = for | | | | | | | |
| 15 | 104710_at | Cluster Incl Y13231: Bcl2 homologous antagonist/killer/cds = (254,880)/gb = Y13231/gi = 2113878/ug = Mm 2443/len = 2005/ | 726.8 | P | 565.6 | P | 632.9 | P | 755 |
| | | STRA = for | | | | | | | |
| 15 | 94341_at | Cluster Incl D31967 Jumonji/cds = (210,3914)/gb = D31967/gi = 780143/ug = Mm.25059/len = 4071/STRA = for | 1384.9 | P | 1306.5 | P | 1355.7 | P | 1478.2 |
| 15 | 94929_at | Cluster Incl M97590: Mouse tyrosine phosphatase (PTP-1), (EC2 7 1.112) mRNA/cds = UNKNOWN/gb = M97590/ | 959.7 | P | 977.3 | P | 1108.5 | P | 1020.3 |
| | | gi = 200581/ug = Mm 2668/len = 1664/STRA = for | | | | | | | |
| 15 | 94972_at | Cluster Incl AB026569 Mus musculus mRNA for MSSP, complete cds/cds = (261,1373)/gb = AB026569/gi = 4730905/ | 604.9 | P | 403.5 | P | 441.1 | P | 631.8 |
| | | ug = Mm 27230/len = 1650/STRA = for | | | | | | | |
| 15 | 95627_at | Cluster Incl AW046038 UI-M-BH1-akv-b-03-0-U1.s1 Mus musculus cDNA, 3 end/clone = UI-M-BH1-akv-b-03-0-UI/ | 561.8 | P | 367 | P | 416.9 | P | 611 |
| | | clone_end = 3/gb = AW046038/gi = 5906567/ug = Mm 28060/len = 433/STRA = for | | | | | | | |
| 15 | 96197_f_at | Cluster Incl AI851708 UI-M-BHO-aio-d-08-0-UI s1 Mus musculus cDNA, 3 end/clone = UI-M-BH0-aio-d-08-0-UI/ | 395.8 | P | 380.4 | P | 385.6 | P | 385.4 |
| | | clone_end = 3/gb = AI851708/gi = 5495609/ug = Mm.28553/len = 337/STRA = for | | | | | | | |
| 15 | 96220_at | Cluster Incl AW123157 UI-M-BH2 1-apd-c-10-0-UI.s1 Mus musculus cDNA, 3 end/clone = UI-M-BH2 1-apd-c-10-0-UI/ | 284.2 | P | 272 | P | 213.6 | P | 344.5 |
| | | clone_end = 3/gb = AW123157/gi = 6098687/ug = Mm 28709/len = 542/STRA = for | | | | | | | |
| 15 | 97375_at | Cluster Incl U70209 Mus musculus polycystic kidney disease 1 protein (Pkd1) mRNA, complete cds/cds = (315,13196)/ | 167.3 | P | 143.3 | P | 159.9 | P | 82.2 |
| | | gb = U70209/gi = 2138182/ug = Mm 2973/len = 14141/STRA = for | | | | | | | |
| 15 | 97426_at | Cluster Incl X98471: Epithelial membrane protein 1/cds = (0,482)/gb = X98471/gi = 1580765/ug = Mm.30024/len = 483/ | 3390.4 | P | 2664.6 | P | 3309.1 | P | 3412.5 |
| | | STRA = for | | | | | | | |
| 15 | 97937_at | Cluster Incl AA611766: vo85108.r1 Mus musculus cDNA, 5 end/clone = IMAGE-1065927/clone_end = 5/gb = AA611766/ | 1267.8 | P | 911 | P | 1232.5 | P | 1112.8 |
| | | gi = 2461801/ug = Mm.30262/len = 161/STRA = rev | | | | | | | |
| 15 | 98432_at | Cluster Incl X99384: M musculus mRNA for paladin gene/cds = (46,2625)/gb = X99384/gi = 1545958/ug = Mm.3437/len = 4055/ | 180.1 | P | 127.3 | P | 137.2 | P | 118.3 |
| | | STRA = for | | | | | | | |
| 15 | 98436_s_at | Cluster Incl U54803 Caspase 3, apoptosis related cysteine protease/cds = (15,848)/gb = U54803/gi = 1518967/ | 228.6 | P | 171.6 | P | 203.1 | P | 245.9 |
| | | ug = Mm 34405/len = 1418/STRA = rev | | | | | | | |
| 15 | 98477_s_at | Cluster Incl L40632 Ankyrin 3, epithelial/cds = (56,5941)/gb = L40632/gi = 710548/ug = Mm 3526/len = 7084/STRA = for | 41.6 | P | 83.9 | P | 87.8 | P | 105.9 |
| 15 | 98984_I _at | Cluster Incl D50430 Mouse mRNA for glycerol-3-phosphate dehydrogenase, complete cds/cds = (96,2279)/gb = D50430/ | 287.1 | P | 341.8 | P | 281.5 | P | 357 |
| | | gi = 1339937/ug = Mm 3711/len = 2410/STRA = for | | | | | | | |
| 15 | 99054_at | Cluster Incl D16141: Lethal giant larvae homolog/cds = (21,3125)/gb = D16141/gi = 414350/ug = Mm 3940/len = 4279/ | 1116 | P | 933.2 | P | 1033.6 | P | 995.2 |
| | | STRA = rev | | | | | | | |
| 15 | 99458_I_at | Cluster Incl X70764 ELKL motif kinase/cds = (93,2417)/gb = X70764/gi = 57919/ug = Mm 4082/len = 2763/STRA = for | 2124 | P | 2508.2 | P | 2729.9 | H | 2369.5 |
| 15 | 99934_at | Cluster Incl M80206 Poliovirus sensitivity/cds = (0,1403)/gb = M80206/gi = 199785/ug = Mm 4341/len = 1404/STRA = for | 592.3 | P | 512.4 | P | 499.3 | P | 579.5 |
| 15 | 100011_at | Cluster Incl AI851658: UI-M-BH0-ain-f-03-0-UI s1 Mus musculus cDNA, 3 end/clone = UI-M-BH0-ain-f-03-0-UI/ | 1639 | P | 1484 | P | 1155.4 | P | 1635 |
| | | clone_end = 3/gb = AI851658/gi = 5495564/ug = Mm 4549/len = 448/STRA = for | | | | | | | |
| 15 | 100027_s_at | Cluster Incl AI881987 uI56h05.x1 Mus musculus cDNA, 3 end/clone = IMAGE-2123097/clone_end = 3/gb = AI881987/ | 650.8 | P | 616.3 | P | 738.2 | P | 625.9 |
| | | gi = 5567139/ug = Mm 4609/len = 512/STRA = for | | | | | | | |
| 15 | 100029_at | Cluster Incl AW208667: uo62e02 x1 Mus musculus cDNA, 3 end/clone = IMAGE-2647130/clone_end = 3/gb = AW208667/ | 1497.3 | P | 1116.5 | P | 1961.5 | P | 1911.1 |
| | | gi = 6514607/ug = Mm 4609/len = 474/STRA = for | | | | | | | |
| 15 | 100032_at | Cluster Incl X60136 Trans-acting transcription factor 1/cds = (0,307)/gb = X60136/gi = 54158/ug = Mm 4618/len = 454/ | 749.6 | P | 413.8 | P | 341.1 | P | 754.9 |
| | | STRA = for | | | | | | | |
| 15 | 100401_at | Cluster Incl AA673981: vo87c03 r1 Mus musculus cDNA, 5 end/clone = IMAGE-1066084/clone_end = 5/gb = AA673981/ | 501.7 | P | 660.5 | P | 665.2 | P | 623.8 |
| | | gi = 2647263/ug = Mm 46401/len = 358/STRA = for | | | | | | | |
| 15 | 100420_at | Cluster Incl J03458 Filaggnn/cds = (0,1010)/gb = J03458/gi = 193307/ug = Mm 4685/len = 1488/STRA = for | 4339.5 | P | 4311.9 | P | 4183.4 | P | 3691.8 |
| 15 | 100514_at | Cluster Incl M63660 Mouse G-alpha-13 protein mRNA, complete cds/cds = (128,1261)/gb = M63660/gi = 193378/ | 737.1 | P | 679 | P | 799.1 | P | 826 |
| | | ug = Mm 5238/len = 2422/STRA = rev | | | | | | | |
| 15 | 101406_at | Cluster Incl AF026072 Mus musculus hydroxysteroid sulfotransferase (SULT2B) mRNA, complete cds/cds = (69,1085)/ | 722.4 | P | 701.2 | P | 719.2 | P | 704.4 |
| | | gb = AF026072/gi = 2570897/ug = Mm 7316/len = 1127/STRA = for | | | | | | | |
| 15 | 101448_at | Cluster Incl D50050: HGF-regulated tyrosine kinase substrate/cds = (42,2369)/gb = D50050/gi = 1089780/ug = Mm.7919/ | 1926.7 | P | 1800.3 | P | 1938.3 | P | 1864.2 |
| | | len = 2458/STRA = for | | | | | | | |
| 15 | 101922_at | Cluster Incl AW123408 UI-M-BH2 1-apj-b-01-0-UI.s1 Mus musculus cDNA, 3 end/clone = UI-M-BH2.1-apj-b-01-0-UI/ | 752.5 | P | 718.5 | P | 710.4 | P | 601.2 |
| | | clone_end = 3/gb = AW123408/gi = 6098938/ug = Mm 92288/len = 410/STRA = for | | | | | | | |
| 15 | 102222_at | Cluster Incl AJ002730 Ubiquitously transcribed tetratricopeptide repeat gene, X Chromosome/cds = (0,4003)/gb = AJ002730/ | 559.4 | P | 334.1 | P | 322.6 | P | 384.2 |
| | | gi = 3021456/ug = Mm 10218/len = 4215/STRA = rev | | | | | | | |
| 15 | 102273_at | Cluster Incl M31226 Mouse immune suppressor factor TJ6 mRNA, complete cds/cds = (18,2585)/gb = M31226/gi = 198460/ | 325.5 | P | 302.3 | P | 336.6 | P | 259.6 |
| | | ug = Mm 1158/len = 2936/STRA = for | | | | | | | |
| 15 | 102691_at | Cluster Incl AI848518 UI-M-AP1-agf-e-02-0-UI s2 Mus musculus cDNA, 3 end/clone = UI-M-AP1-agf-e-02-0-UI/ | 639.2 | P | 782.7 | P | 891.7 | P | 763.1 |
| | | clone _end _3/gb = AI848518/gi = 5492424/ug = Mm.14099/len = 361/STRA = for | | | | | | | |
| 15 | 102727_at | Cluster Incl X55573 Brain derived neurotrophic factor/cds = (77,826)/gb = X55573/gi = 287898/ug = Mm 1442/len = 927/ | 228 | P | 172.1 | P | 113.2 | P | 111.3 |
| | | STRA = for | | | | | | | |
| 15 | 103269_f_at | Cluster Incl AJ005350 Mus musculus of ZT2 gene encoding zinc finger protein 125/cds = (0,541)/gb = AJ005350/ | 290.1 | P | 117.4 | P | 81.3 | P | 231.9 |
| | | gi = 3646358/ug = Mm.20857/len = 2440/STRA = for | | | | | | | |
| 15 | 103288_at | Cluster Incl AF053062 Mus musculus receptor interacting protein 140 mRNA, complete cds/cds = (371,3856)/ | 433.8 | P | 243.8 | P | 296.7 | P | 363.7 |
| | | gb = AF053062/gi = 3820493/ug = Mm 20895/len = 4307/STRA = for | | | | | | | |
| 15 | 103484_at | Cluster Incl AA619442 vo84c10.r1 Mus musculus cDNA, 5 end/clone = IMAGE-1065810/clone_end = 5/gb = AA619442/ | 7 | P | 54.2 | P | 31.5 | P | 37.4 |
| | | gi = 2523318/ug = Mm.22133/len = 464/STRA = for | | | | | | | |
| 15 | 103709_at | Cluster Incl AA763466 vw54f05 r1 Mus musculus cDNA, 5 end/clone = IMAGE-1247649/clone_end = 5/gb = AA763466/ | 2497.5 | P | 3420 | P | 3151.7 | P | 2829.7 |
| | | gi = 2813213/ug = Mm 24093/len = 379/STRA = for | | | | | | | |
| 15 | 103771_at | Cluster Incl AI837993 UI-M-AP0-abl-d-01-0-UI s1 Mus musculus cDNA, 3 end/clone = UI-M-AP0-abl-d-01-0-UI/ | 1195 | P | 1017.8 | P | 1312.8 | P | 1389.6 |
| | | clone_end = 3/gb = AI837993/gi = 54722067/ug = Mm 24777/len = 336/STRA = rev | | | | | | | |
| 15 | 103774_at | Cluster Incl AW108350: um29f06 x1 Mus musculus cDNA, 3 end/clone = IMAGE-2235971/clone_end = 3/gb = AW108350/ | 422.9 | P | 267.8 | P | 300.5 | P | 387.4 |
| | | gi = 6079150/ug = Mm 24790/len = 579/STRA = for | | | | | | | |
| 15 | 103978_at | Cluster Incl AA666669: vm50f06.r1 Mus musculus cDNA, 5 end/clone = IMAGE-1001699/clone_end = 5/gb = AA666669/ | 543.2 | P | 482.8 | P | 462.5 | P | 606.6 |
| | | gi = 2625370/ug = Mm 25787/len = 412/STRA = for | | | | | | | |
| 15 | 103994_at | Cluster Incl AI152867: ud93h11.r1 Mus musculus cDNA, 5 end/clone = IMAGE-1478469/clone_end = 5/gb = AI152867/ | 875.8 | P | 723.8 | P | 746.1 | P | 891 |
| | | gi = 3681336/ug = Mm 26266/len = 471/STRA = for | | | | | | | |
| 15 | 104158_at | Cluster Incl AW046671: UI-M-BH1-alm-a-09-0-UI s1 Mus musculus cDNA, 3 end/clone = UI-M-BH1-alm-a-09-0-UI/ | 205 | P | 114.5 | P | 80.3 | P | 209 |
| | | clone_end = 3/gb = AW046671/gi = 5907200/ug = Mm.27094/len = 240/STRA = for | | | | | | | |
| 15 | 104225_at | Cluster Incl AI645050 vs46d05 y1 Mus musculus cDNA, 5 end/clone = IMAGE-1149321/clone_end = 5/gb = AI645050/ | 532.2 | P | 337.8 | P | 669.4 | P | 533.3 |
| | | gi = 4723525/ug = Mm 27988/len = 373/STRA = rev | | | | | | | |
| 15 | 104407_at | Cluster Incl L25274 Mus musculus transmembrane glycoprotein (DM-GRASP) mRNA, 3 end/cds = (0,1075)/gb = L25274/ | 398.6 | P | 240.8 | P | 192.5 | P | 333.8 |
| | | gi = 452102/ug = Mm 2877/len = 1288/STRA = for | | | | | | | |
| 15 | 104433_at | Cluster Incl AI849682 UI-M-AL1-ahl-f-01-0-UI s1 Mus musculus cDNA, 3 end/clone = UI-M-AL1-ahl-f-01-0-UI/clone_end = 3/ | 489.1 | P | 322.9 | P | 374.7 | P | 437.2 |
| | | gb = AI849682/gi = 5493588/ug = Mm 29228/len = 245/STRA = rev | | | | | | | |
| 15 | 104461_at | Cluster Incl AW121773 UI-M-BH2 3-aoe-f-11-0-UI s1 Mus musculus cDNA, 3 end/clone = UI-M-BH2.3-aoe-f-11-0-UI/ | 165.9 | P | 232.3 | P | 223.4 | P | 218.7 |
| | | clone_end = 3/gb = AW121773/gi = 6097121/ug = Mm 29589/len = 166/STRA = for | | | | | | | |
| 15 | 104663_at | Cluster Incl D86177 Mouse mRNA for phosphatidylinositol 4-phosphate 5-kinase type l-beta, complete cds/cds = (269,1909)/ | 350.4 | P | 253.1 | P | 391 | P | 489.6 |
| | | gb = D86177/gi = 1622709/ug = Mm 3191/len = 2403/STRA = for | | | | | | | |
| 15 | 104682_at | Cluster Incl AI848983: UI-M-AJ1-ahd-d-05-0-UI s1 Mus musculus cDNA, 3 end/clone = UI-M-AJ1-ahd-d-05-0-UI/ | 937.1 | P | 727.4 | P | 712.1 | P | 911.7 |
| | | clone_end = 3/gb = AI848983/gi = 5492889/ug = Mm.32884/len = 499/STRA = for | | | | | | | |
| 15 | 92195_at | Cluster Incl AB012273 Mus musculus mRNA for GPE1-BP (C/EBP gamma), complete cds/cds = (224,676)/gb = AB012273/ | 372.9 | P | 263 | P | 222.3 | P | 276.7 |
| | | gi = 2988367/ug = Mm 3402/len = 1185/STRA = for | | | | | | | |
| 15 | 92444_f_at | Cluster Incl X16493: Zinc finger protein 1/cds = (320,1594)/gb = X16493/gi = 55482/ug = Mm.4184/len = 1939/STRA = rev | 262.3 | P | 218.3 | P | 181.4 | P | 220.5 |
| 15 | 92452_at | Cluster Incl U03279: Phosphatidylinositol 3-kinase, catalytic, alpha polypeptide/cds = (0,3206)/gb = U03279/gi = 414994/ | 185.8 | P | 163.3 | P | 143.8 | P | 161.8 |
| | | ug = Mm 41943/len = 3207/STRA = rev | | | | | | | |
| 15 | 92703_at | Cluster Incl AI325791 mn19e09.y1 Mus musculus cDNA, 5 end/clone = IMAGE-538408/clone_end = 5/gb = AI325791/ | 337.2 | P | 142.5 | P | 175.4 | P | 251.8 |
| | | gi = 4060220/ug = Mm 45992/len = 434/STRA = for | | | | | | | |
| 15 | 92891_f_at | Cluster Incl X55318 Homeo box C9/cds = (348,1130)/gb = X55318/gi = 57878/ug = Mm 4765/len = 1434/STRA = for | 63.6 | P | 148.6 | P | 70.9 | P | 104.9 |
| 15 | 92903_at | Cluster Incl X78197 Transcription factor AP-2 beta/cds = (213,1559)/gb = X78197/gi = 496638/ug = Mm 4795/len = 1731/ | 291.5 | P | 454 | P | 318.8 | P | 345.2 |
| | | STRA = for | | | | | | | |
| 15 | 92918_at | Cluster Incl U66079 Mus musculus coagulation factor VII gene, complete cds/cds = (10,1350)/gb = U66079/gi = 1513221/ | 167.7 | P | 188.5 | P | 164.1 | P | 153.1 |
| | | ug = Mm.4827/len = 1843/STRA = for | | | | | | | |
| 15 | 93378_at | Cluster Incl X07439 Mouse mRNA for Hox-3 1 homeo gene/cds = (241,969)/gb = X07439/gi = 51406/ug = Mm 6167/ | 664.9 | P | 808 | P | 527.9 | P | 666.6 |
| | | len = 1470/STRA = for | | | | | | | |
| 15 | 93478_at | Cluster Incl AA612483: vo72g09.r1 Mus musculus cDNA, 5 end/clone = IMAGE-1064704/clone_end = 5/gb = AA612483/ | 265.2 | P | 187 | P | 185.4 | P | 191 |
| | | gi = 2461161/ug = Mm.7109/len = 384/STRA = rev | | | | | | | |
| 15 | 93938_at | Cluster Incl U17132: Zinc transporter 1/cds = (147,1658)/gb = U17132/gi = 577840/ug = Mm.9024/len = 1720/STRA = for | 232.9 | P | 354.9 | P | 409.7 | P | 361.4 |
| 15 | 94127_at | Cluster Incl Y15910 Mus musculus mRNA for dia protein/cds = (0,1049)/gb = Y15910/gi = 3171941/ug = Mm.10763/len = 1330/ | 128.6 | P | 91.2 | P | 89.7 | P | 177 |
| | | STRA = for | | | | | | | |
| 15 | 94689_at | Cluster Incl C79248 C79248 Mus musculus cDNA, 3 end/clone = J0063C04/clone_end = 3/gb = C79248/gi = 2519578/ | 660.2 | P | 690 | P | 661 | P | 578.2 |
| | | ug = Mm 13291/len = 476/STRA = rev | | | | | | | |
| 15 | 94767_at | Cluster Incl AB028894 Mus musculus Rps11, U35 genes for ribosomal protein S11 and U35 snoRNA, complete cds and | 4279.2 | P | 3315.5 | P | 3848.5 | P | 3937 |
| | | sequence/cds = (22,498)/gb = AB028894/gi = 6552367/ug = Mm 164/len = 551/STRA = for | | | | | | | |
| 15 | 95305_at | Cluster Incl AA756275 vv40d02 r1 Mus musculus cDNA, 5 end/clone = IMAGE-1224867/clone_end = 5/gb = AA756275/ | 269.3 | P | 323.1 | P | 245.1 | P | 282 |
| | | gi = 2803473/ug = Mm 20012/len = 526/STRA = for | | | | | | | |
| 15 | 95359_at | Cluster Incl M18186 Mouse 84 kD heat shock protein mRNA, complete cds/cds = (94,2268)/gb = M18186/gi = 194024/ | 2332.9 | P | 2220.7 | P | 2400.9 | P | 2603.9 |
| | | ug = Mm 2180/len = 2509/STRA = for | | | | | | | |
| 15 | 95385_at | Cluster Incl AI643119 mm55a03 y1 Mus musculus cDNA, 5 end/clone = IMAGE-532300/clone_end = 5/gb = AI643119/ | 454.7 | P | 480.7 | P | 478.2 | P | 439.5 |
| | | gi = 4721594/ug = Mm 23210/len = 285/STRA = rev | | | | | | | |
| 15 | 95393_at | Cluster Incl AI503362: vn86g08.x1 Mus musculus cDNA, 3 end/clone = IMAGE-1038878/clone_end = 3/gb = AI503362/ | 293.2 | P | 204.9 | P | 199.6 | P | 329.6 |
| | | gi = 4401213/ug = Mm 23817/len = 425/STRA = for | | | | | | | |
| 15 | 95944_at | Cluster Incl AV299153 AV299153 Mus musculus cDNA, 3 end/clone = 5730466C10/clone_end = 3/gb = AV299153/ | 431.8 | P | 279.4 | P | 180.2 | P | 455.5 |
| | | gi = 6331610/ug = Mm 24891/len = 273/STRA = rev | | | | | | | |
| 15 | 96522_at | Cluster Incl M19381 Calmodulin/cds = (186,635)/gb = M19381/gi = 469421/ug = Mm.2648/len = 798/STRA = for | 4158.7 | P | 3721.7 | P | 3665.8 | P | 3968.2 |
| 15 | 96532_at | Cluster Incl AA212897: mw62a09 r1 Mus musculus cDNA, 5 end/clone = lMAGE-677176/clone_end = 5/gb = AA212897/ | 146 | P | 113.9 | P | 96.3 | P | 160.9 |
| | | gi = 1811645/ug = Mm 27297/len = 429/STRA = for | | | | | | | |
| 15 | 96542_at | Cluster Incl M62606 Surfeit gene 4/cds = (105,914)/gb = M62606/gi = 201091/ug = Mm 2795/len = 2781/STRA = for | 1608.5 | P | 2065.7 | P | 1961.5 | P | 1755.8 |
| 15 | 96573_at | Cluster Incl M21495 Mouse cytoskeletal gamma-actin mRNA, complete cds/cds = (39,1186)/gb = M21495/gi = 191656/ | 6421.1 | P | 5698.2 | P | 5923.9 | P | 5797.6 |
| | | ug = Mm 29913/1en = 1868/STRA = for | | | | | | | |
| 15 | 96575_at | Cluster Incl U67771: Mus musculus ribosomal protein L8 (RPL8) mRNA, complete cds/cds = (21,794)/gb = U67771/ | 3704.3 | P | 2577.4 | P | 3200.7 | P | 3325.4 |
| | | gi = 1527177/ug = Mm 30066/len = 842/STRA = for | | | | | | | |
| 15 | 97695_s_at | Cluster Incl M29015.Mouse ribosomal protein L7 (rpL7) gene, complete cds/cds = (0,812)/gb = M29015/gi = 200784/ | 6376.2 | P | 5494 | P | 5793.7 | P | 5954.6 |
| | | ug = Mm 37835/len = 813/STRA = for | | | | | | | |
| 15 | 97696_r_at | Cluster Incl M29015 Mouse ribosomal protein L7 (rpL7) gene, complete cds/cds = (0,812)/gb = M29015/gi = 200784/ | 2951.1 | P | 2779.1 | P | 2927.8 | P | 3086.9 |
| | | ug = Mm 37835/len = 813/STRA = for | | | | | | | |
| 15 | 97750_at | Cluster Incl X06406 Mouse mRNA for tranalational controlled 40 kDa polyyeptide p40/cds = (77,964)/gb = X06406/gi = 52852/ | 2188.3 | P | 2128.6 | P | 2079 | P | 2178 |
| | | ug = Mm 4071/len = 1031/STRA = for | | | | | | | |
| 15 | 97751_f_at | Cluster Incl AI835771 UI-M-AI0-aaq-a-05-0-UI s1 Mus musculus cDNA, 3 end/clone = UI-M-AI0-aaq-a-05-0-UI/ | 1852.9 | P | 1901.7 | P | 1845.2 | P | 1971.4 |
| | | clone_end = 3/gb = AI835771/gi = 5469984/ug = Mm 40749/len = 324/STRA = rev | | | | | | | |
| 15 | 98313_at | Cluster InCL AB025413 Mus musculus mRNA for Ten-m4, complete cds/cds = (187,8502)/gb = AB025413/gi = 4760781/ | 160.5 | P | 232.5 | P | 145.9 | P | 157.9 |
| | | ug = Mm 42204/len = 8585/STRA = for | | | | | | | |
| 15 | 98319_at | Cluster Incl AJ000328 Mus musculus mRNA tor type 2 desmoglein/cds = (0,245)/gb = AJ000328/gi = 2764500/ | 214.7 | P | 167.4 | P | 149.8 | P | 202.4 |
| | | ug = Mm 42229/len = 745/STRA = for | | | | | | | |
| 15 | 98331_at | Cluster Incl X52046: Procollagen, type III, alpha 1/cds = (117,4511)/gb = X52046/gi = 575321/ug = Mm 4271/len = 4766/ | 4833.8 | P | 4935.3 | P | 4627.6 | P | 5023.4 |
| | | STRA = for | | | | | | | |
| 15 | 98333_at | Cluster Incl AF100956 Mus musculus major histocompatibility locus class II region, Fas-binding protein Daxx (DAXX) gene, | 3857.5 | P | 2340.2 | P | 2539.5 | P | 3194.2 |
| | | partial cds, Bing1 (BING1), tapasin (tapasin), RalGDS-like factor (RLF), KE2 (KE2), BING4 (BING4), beta1, 3-galactosyl | | | | | | | |
| | | transferase (be/STRA = for | | | | | | | |
| 15 | 98342_at | Cluster Incl M93980 Mouse 24.6 kda protein mRNA, complete cds/cds = (41,685)/gb = M93980/gi = 410741/ug = Mm 43239/ | 4531.6 | P | 3701.2 | P | 3975.1 | P | 4287.9 |
| | | len = 757/STRA = for | | | | | | | |
| 15 | 98346_at | Cluster Incl AI593759 v171b05 x1 Mus musculus cDNA, 3 end/clone = IMAGE-1176561/clone_end = 3/gb = AI593759/ | 176.6 | P | 177.1 | P | 253.2 | P | 159.4 |
| | | gi = 4602807/ug = Mm 43514/len = 296/STRA = for | | | | | | | |
| 15 | 98369_f_at | Cluster Incl AW048053: UI-M-BH1-alv-g-06-0-U1.s1 Mus musculus cDNA, 3 end/clone = UI-M-BH1-alv-g-06-0-UI/ | 3237.8 | P | 3150 | P | 3223.3 | P | 3024.9 |
| | | clone_end = 3/gb = AW048053/gi = 5908582/ug = Mm 44493/len = 532/STRA = rev | | | | | | | |
| 15 | 98759_f_at | Cluster Incl M28727 Tubulin alpha 2/cds = (0,1058)/gb = M28727/gi = 202224/ug = Mm 4591/len = 1198/STRA = for | 2940.4 | P | 2511.4 | P | 2813.7 | P | 2997 |
| 15 | 98767_at | Cluster Incl AI842603: UI-M-AQ1-aed-b-11-0-UI s1 Mus musculus cDNA, 3 end/clone = UI-M-AQ1-aed-b-11-0-UI/ | 275.6 | P | 274.5 | P | 227 | P | 223.3 |
| | | clone_end = 3/gb = AI842603/gi = 5476818/ug = Mm 463/len = 349/STRA = rev | | | | | | | |
| 15 | 98817_at | Cluster Incl Z29532 M musculus mRNA for follistatin/cds = (0,1031)/gb = Z29532/gi = 488368/ug = Mm 4913/len = 1123/ | 422.2 | P | 416.1 | P | 600 | P | 489.9 |
| | | STRA = for | | | | | | | |
| 15 | 98862_at | Cluster Incl U61969 Mus musculus Wn110a mRNA, complete cds/cds = (662,1915)/gb = U61969/gi = 1546012/ug = Mm 5130/ | 297.2 | P | 245.1 | P | 324.2 | P | 267.5 |
| | | len = 2487/STRA = rev | | | | | | | |
| 15 | 99327_at | Cluster Incl D30785 Mouse mRNA for neuropsin, complete cds/cds = (486,1268)/gb = D30785/gi = 1648847/ug = Mm 5193/ | 730.2 | P | 785.4 | P | 862.8 | P | 859.4 |
| | | len = 1322/STRA = for | | | | | | | |
| 15 | 99335_at | Cluster Incl J05277: Hexokinase 1/cds = (4,2760)/gb = J05277/gi = 193841/ug = Mm 5290/len = 4198/STRA = for | 3673.5 | P | 3385 | P | 3484.4 | P | 3480.2 |
| 15 | 99336_at | Cluster Incl U78085: Mus musculus ribosomal protein S5 mRNA, complete cds/cds = (65,679)/gb = U78085/gi = 1685070/ | 4939 | P | 4485.1 | P | 4577.7 | P | 4549.4 |
| | | ug = Mm.5291/len = 763/STRA = for | | | | | | | |
| 15 | 99340_at | Cluster Incl D29802 Guanine nucleotide binding protein, beta-2, related sequence 1/cds = (98,1051)/gb = D29802/ | 4964.1 | P | 3970.3 | P | 4149.6 | P | 4593.4 |
| | | gi = 475011/ug = Mm 5305/len = 1092/STRA = for | | | | | | | |
| 15 | 100297_at | Cluster Incl AA693125 vr57c08 s1 Mus musculus cDNA, 5 end/clone = IMAGE-1124750/clone_end = 5/gb = AA693125/ | 643.9 | P | 636.4 | P | 894.9 | P | 1010.4 |
| | | gi = 2694063/ug = Mm 86411/len = 265/STRA = for | | | | | | | |
| 15 | 100307_at | Cluster Incl AA002843 mg38c08 r1 Mus musculus cDNA, 5 end/cone = IMAGE-426062/clone_end = 5/gb = AA002343/ | 188.9 | P | 153.5 | P | 157.5 | P | 136.3 |
| | | gi = 1446281/ug = Mm 86787/len = 235/STRA = for | | | | | | | |
| 15 | 100343_f_at | Cluster Incl M28729 Tubulin alpha1/cds = (0,596)/gb = M28729/gi = 202222/ug = Mm 88110/len = 786/STRA = for | 3577.3 | P | 3421.7 | P | 3412.2 | P | 3479.7 |
| 15 | 100379_f_at | Cluster Incl AI837905 UI-M-AL0-abq-c-01-0-UI s1 Mus musculus cDNA, 3 end/clone = UI-M-AL0-abq-c-01-0-UI/ | 4250.2 | P | 3786.7 | P | 3732.2 | P | 3812.7 |
| | | clone_end = 3/gb = AI837905/gi = 5472118/ug = Mm.89082/len = 223/STRA = rev | | | | | | | |
| 15 | 100380_at | Cluster Incl X91866 M musculus mRNA for H3 3A variant histone/cds = UNKNOWN/gb = X91866/gi = 1022324/ | 4088.3 | P | 3922.7 | P | 3687.2 | P | 3266 |
| | | ug = Mm 89136/len = 1304/STRA = for | | | | | | | |
| 15 | 100758_at | Cluster Incl AF110520 Mus musculus major histocompatibility complex region NG27, NG28, RPS28, NADH | 3912.2 | P | 3332.8 | P | 3593.5 | P | 3685 |
| | | oxidoreductase, NG29, KIFC1, Fas-binding protein, BING1, tapasin, RalGDS-like, KE2, BING4, beta 1,3-galactosyl | | | | | | | |
| | | transferase, and RPS18 genes, complete cds, S/STRA = for | | | | | | | |
| 15 | 100780_at | Cluster Incl M73436 Ribosomal protein S4, X-linked/cds = (6,797)/gb = M73436/gi = 200863/ug = Mm.66/len = 885/STRA = for | 5420.8 | P | 4600.1 | P | 4973.7 | P | 5161.6 |
| 15 | 101255_at | Cluster Incl X51703 Mouse mRNA for ubiquitin/cds = (104,1021)/gb = X51703/gi = 55117/ug = Mm.235/len = 1172/STRA = for | 3867 | P | 3088.7 | P | 2691 | P | 3423.7 |
| 15 | 101732_at | Cluster Incl M12039 Mouse DNA homologous to the Drosophila per locus/cds = (0,2018)/gb = M12039/gi = 200288/ | 479.5 | P | 488.8 | P | 469 | P | 446.3 |
| | | ug = Mm 57054/len = 2019/STRA = for | | | | | | | |
| 15 | 101781_f_at | Cluster Incl V00754 Mouse gene coding for embryonic H3 histone/cds = (0,407)/gb = V00754/gi = 51297/ug = Mm 57118/ | 1414.5 | P | 1234.9 | P | 1301.6 | P | 1178.8 |
| | | len = 408/STRA = for | | | | | | | |
| 15 | 101787_f_at | Cluster Incl X16672: Mouse DNA for type IIB intracistemal A-particle element encoding integrase and gag/cds = (0,323)/ | 3383.9 | P | 3341.6 | P | 3218.6 | P | 3304.9 |
| | | gb = X16672/gi = 51499/ug = Mm 57126/len = 324/STRA = for | | | | | | | |
| 15 | 102599_at | Cluster Incl X06407 Mouse mRNA for 21 kd polypeptide under translational control/cds = (100,618)/gb = X06407/gi = 52848/ | 3441.1 | P | 2405.8 | P | 2928.2 | P | 3046.4 |
| | | ug = Mm 254/len = 816/STRA = for | | | | | | | |
| 15 | 94662_at | Cluster Incl AA409766 EST01475 Mus musculus cDNA, 3 end/clone = C0010D03/clone_end = 3/gb = AA409766/ | 436.3 | P | 434.1 | P | 392.3 | P | 312.9 |
| | | gi = 2066390/ug = Mm 24471/len = 316/STRA = rev | | | | | | | |
| 15 | 95184_f_at | Cluster Incl AA419684 vc51g02 r1 Mus musculus cDNA, 3 end/clone = IMAGE-778130/clone_end = 3/gb = AA419684/ | 416.3 | P | 376.3 | P | 312.1 | P | 409.3 |
| | | gi = 2080962/ug = Mm 24548/len = 243/STRA = rev | | | | | | | |
| 15 | 96962_at | Cluster Incl X81987 M musculus mRNA for TAX responsive element binding protein 107/cds = (27,890)/gb = X81987/ | 4141 | P | 3464 | P | 3637.9 | P | 3810.9 |
| | | gi = 695637/ug = Mm 588/len = 950/STRA = for | | | | | | | |
| 15 | 97017_f_at | Cluster Incl AW214439 uo48c01 X1 Mus musculus cDNA, 3 end/clone = IMAGE-2645760/clone_end = 3/gb = AW214439/ | 1915.8 | P | 1737.3 | P | 1720.7 | P | 1717.4 |
| | | gi = 6520962/ug = Mm 89021/len = 650/STRA = rev | | | | | | | |
| 15 | 97077_f_at | Cluster Incl AW213991 uo46d02 x1 Mus musculus cDNA, 3 end/clone = IMAGE-2645571/clone_end = 3/gb = AW213991/ | 1635.1 | P | 968.6 | P | 1601.5 | P | 1459.3 |
| | | gi = 6520344/ug = Mm B9381/len = 468/STRA = rev | | | | | | | |
| 15 | 97559_at | Cluster Inc M76131 Mouse elongation factor2 (ef-2) mRNA, 3 end/cds = (0,806)/gb = M76131/gi = 192988/ug = Mm 70787/ | 3847.7 | P | 3580.6 | P | 3635.6 | P | 3759.5 |
| | | len = 1179/STRA = for | | | | | | | |
| 15 | 97594_r_at | Cluster Incl C79694.C79694 Mus musculus cDNA, 3 end/clone = J0070F03/clone_end = 3/gb = C79694/gi = 2520024/ | 1074.1 | P | 952.5 | P | 1032.4 | P | 1384.6 |
| | | ug = Mm 89577/len = 439/STRA = rev | | | | | | | |
| 15 | 97647_at | Cluster Incl M11408 Ribosomal protein S16/cds = (52,489)/gb = M11408/gi = 435544/ug = Mm 702/len = 547/STRA = for | 4586.5 | P | 3650.7 | P | 4135.1 | P | 4108.1 |
| 15 | 98168_at | Cluster Incl M14689 Mouse surfeit locus surfeit 3 protein gene/cds = (0,800)/gb = M14689/gi = 201081/ug = Mm.725/len = 801/ | 3106.2 | P | 3216.1 | P | 3339.5 | P | 3450.4 |
| | | STRA = for | | | | | | | |
| 15 | 98169_s_at | Cluster Incl AU020229 AU020229 Mus musculus cDNA, 3 end/clone = J0529B09/clone_end = 3/gb = AU020229/ | 288.9 | P | 344.2 | P | 266.2 | P | 254.7 |
| | | gi = 3375813/ug = Mm 26206/len = 442/STRA = rev | | | | | | | |
| 15 | 98254_f_at | Cluster Incl AW209004 un98106.x1 Mus musculus cDNA, 3 end/clone = IMAGE-2581955/clone_end = 3/gb = AW209004/ | 4089.3 | P | 3927.6 | P | 3545.1 | P | 3760 |
| | | gi = 6514944/ug = Mm 91451/len = 693/STRA = rev | | | | | | | |
| 15 | 100685_at | Cluster Incl M10937: Keratin complex 2, basic, gene 1/cds = (67,1953)/gb = M10937/gi = 4159805/ug = Mm.14245/len = 2341/ | 2160.1 | P | 1455.1 | P | 1490.5 | P | 2154.7 |
| | | STRA = for | | | | | | | |
| 15 | 100694_at | Cluster Incl U29402 Mus musculus acidic ribosomal phosphoprotein P1 mRNA, complete cds/cds = (110,454)/gb = U29402/ | 2806.5 | P | 2241.3 | P | 2526.3 | P | 2718.7 |
| | | gi = 902557/ug = Mm 14843/len = 495/STRA = for | | | | | | | |
| 15 | 100710_at | Cluster Incl Z14044 Valosin containing protein/cds = (161,2581)/gb = Z14044/gi = 55216/ug = Mm 18921/len = 3143/ | 2658.6 | P | 2838.3 | P | 2687 | P | 2932.7 |
| | | STRA = for | | | | | | | |
| 15 | 100711_at | Cluster Incl U12403 Mus musculus Csa-19 mRNA, complete cds/cds = (15,668)/gb = U12403/gi = 531168/ug = Mm 2424/ | 4252.9 | P | 3932.2 | P | 3991.6 | P | 4237.9 |
| | | len = 700/STRA = for | | | | | | | |
| 15 | 100718_at | Cluster Incl X56135: Mouse mRNA for prothymosin alpha/cds = (170,505)/gb = X56135/gi = 54796/ug = Mm.19187/len = 1191/ | 6391.9 | P | 6375.1 | P | 6793 | P | 6577.6 |
| | | STRA = for | | | | | | | |
| 15 | 100720_at | Cluster Incl X65553 M. musculus mRNA for poly(A) binding protein/cds = (253,2163)/gb = X65553/gi = 53753/ug = Mm 2642/ | 4131.1 | P | 3580.4 | P | 4020.8 | P | 4137 |
| | | len = 2244/STRA = for | | | | | | | |
| 15 | 100727_at | Cluster Incl X74856 M. musculus L28 mRNA for ribosomal protein L28/cds = (70,483)/gb = X74856/gl = 488834/ug = Mm 3111/ | 3962.7 | P | 3342.9 | P | 3396.9 | P | 3693.5 |
| | | len = 555/STRA = for | | | | | | | |
| 15 | 100729_at | Cluster Incl X80699: M. musculus L26 mRNA/cds = (47,484)/gb = X80699/gi = 565623/ug = Mm 3229/len = 545/STRA = for | 3954.1 | P | 3136.5 | P | 3424.8 | P | 3703.8 |
| 15 | 100732_at | Cluster Incl X73829.M. musculus mRNA for ribosomal protein S8/cds = (6,632)/gb = X73829/gi = 313297/ug = Mm 3381/ | 5399.8 | P | 5175.1 | P | 5262.3 | P | 5201.5 |
| | | len = 671/STRA = for | | | | | | | |
| 15 | 100734_at | Cluster Incl Y00225 Murine mRNA for J1 protein, yeast ribosomal protein L3 homologue/cds = (9,1220)/gb = Y00225/ | 5010.3 | P | 4638.8 | P | 4756.7 | P | 4934.9 |
| | | gi = 52740/ug = Mm.3486/len = 1276/STRA = for | | | | | | | |
| 15 | 100753_at | Cluster Incl L01062 Mouse ATP synthase alpha subunit, complete cds/cds = (38,1699)/gb = L01062/gi = 192089/ | 4067.9 | P | 3822 | P | 3831 | P | 3853.4 |
| | | ug = Mm 4069/len = 2182/STRA = for | | | | | | | |
| 15 | 101128_at | Cluster Incl L06234: Mus musculus dihydropyridine sensitive skeletal muscle calcium channel mRNA, complete cds/ | 169.4 | P | 261.8 | P | 344.7 | P | 200.9 |
| | | cds = (116,4171)/gb = L06234/gi = 192323/ug = Mm.4418/len = 6010/STRA = for | | | | | | | |
| 15 | 101129_at | Cluster Incl X83590: M. musculus mRNA for ribosomal protein L5, 3 end/cds = (0,278)/gb = X83590/gi = 619503/ug = Mm.4419/ | 2700.9 | P | 2765.1 | P | 2599.3 | P | 2601.2 |
| | | len = 349/STRA = for | | | | | | | |
| 15 | 101130_at | Cluster Incl X58251 Procollagen, type I, alpha 2/cds = (79,4200)/gb = X58251/gi = 50488/ug = Mm 4482/len = 4270/STRA = for | 3856.7 | P | 4082.4 | P | 4122.5 | P | 4107.4 |
| 15 | 101137_at | Cluster Incl X76772: M musculus mRNA for ribosomal protein S3/cds = (5,736)/gb = X76772/gi = 439521/ug = Mm 4696/ | 3117.8 | P | 2863.5 | P | 3138.2 | P | 3157.1 |
| | | len = 818/STRA = for | | | | | | | |
| 15 | 101165_at | Cluster Incl W91762 MTA D08 085 A Mus musculus cDNA, 3 end/clone = MTA D08 085/clone_end = 3/gb = W91762/ | 1297.8 | P | 1180 | P | 1185.4 | P | 1225.7 |
| | | gi = 1407931/ug = Mm 5000/len = 231/STRA = rev | | | | | | | |
| 15 | 101179_at | Cluster Incl D50494.D-E-A-D (aspartate-glutamate-alanine-aspartate) box polypeptide 6/cds = (258,1709)/gb = D50494/ | 2120.9 | P | 2199.6 | P | 2112.3 | P | 2142.2 |
| | | gi = 940407/ug = Mm 5082/len = 2710/STRA = for | | | | | | | |
| 15 | 101207_at | Cluster Incl X52803 Mouse mRNA for cyclophilin (EC 5.2.1.8)/cds = (41,535)/gb = X52803/gi = 50620/ug = Mm 5246/len = 736/ | 5386.1 | P | 4717.8 | P | 5740.4 | P | 5556.7 |
| | | STRA = for | | | | | | | |
| 15 | 101212_at | Cluster Incl AF043285 Mus musculus ribosomal protein S7 (rpS7) gene, complete cds/cds = (79,663)/gb = AF043285/ | 4030.6 | P | 3216.3 | P | 3423 | P | 3587.1 |
| | | gi = 2811283/ug = Mm 5281/len = 682/STRA = for | | | | | | | |
| 15 | 101214_f_at | Cluster Incl M32599 Glyceraldehyde-3-phosphate dehydrogenase/cds = (46,1047)/gb = M32599/gi = 193423/ug = Mm 5289/ | 3066.4 | P | 3742.3 | P | 3621.6 | P | 3531.1 |
| | | len = 1228/STRA = for | | | | | | | |
| 15 | 101664_at | Cluster Incl Z83368 M musculus RPS3a gene/cds = (25,819)/gb = Z83368/gi = 1841931/ug = Mm 6957/len = 864/STRA = for | 5387.9 | P | 4573.9 | P | 5327.2 | P | 5247.5 |
| 15 | 101680_at | Cluster Incl X05021 Munne mRNA with homology to yeast L29 ribosomal protein gene/cds = (22,468)/gb = X05021/ | 4583.9 | P | 5773.2 | P | 4614 | P | 4757.2 |
| | | gi = 50320/ug = Mm 7277/len = 507/STRA = for | | | | | | | |
| 15 | 102098_at | Cluster Incl AI415065 mb90h11.x1 Mus musculus cDNA, 3 end/clone = IMAGE-336741/clone_end = 3/gb = AI415065/ | 4058.8 | P | 3607.2 | P | 3665.1 | P | 3865.2 |
| | | gi = 4258569/ug = Mm 42402/len = 278/STRA = rev | | | | | | | |
| 15 | 102109_at | Cluster Incl U28917 Mus musculus 60S ribosomal protein (A52) mRNA, complete cds/cds = (20,661)/gb = U28917/ | 3646.6 | P | 2866.2 | P | 3236.6 | P | 3519.2 |
| | | gi = 899444/ug = Mm 42578/len = 704/STRA = for | | | | | | | |
| 15 | 102126_at | Cluster Incl X15962 Mouse mRNA for ribosomal protein S12/cds = (56,454)/gb = X15962/gi = 54005/ug = Mm 43452/len = 470/ | 3584.7 | P | 3468.5 | P | 3337.2 | P | 3239.9 |
| | | STRA = for | | | | | | | |
| 15 | 92545_f_at | Cluster Incl AB006361.Mus musculus mRNA for prostaglandin D synthetase, complete cds/cds = (0,629)/gb = AB006361/ | 326 | P | 287 | P | 158.6 | P | 246.8 |
| | | gi = 2317285/ug = Mm.1008/len = 789/STRA = for | | | | | | | |
| 15 | 94241_at | Cluster Incl AI837229 UI-M-AK0-ade-e-06-0-UI.s1 Mus musculus cDNA, 3 end/clone = UI-M-AK0-ade-e-06-0-UI/ | 133.6 | P | 156.3 | P | 169.6 | P | 158.1 |
| | | clone_end = 3/gb = AI837229/gi = 5471482/ug = Mm.2235/len = 492/STRA = for | | | | | | | |
| 15 | 97318_at | Cluster Incl AI846197 UI-M-AM1-agb-e-02-0-UI s1 Mus musculus cDNA, 3 end/clone = UI-M-AM1-agb-e-02-0-UI/ | 120.1 | P | 116.4 | P | 62.9 | A | 95.8 |
| | | clone_end = 3/gb = AI846197/gi = 5490103/ug = Mm 28109/len = 310/STRA = for | | | | | | | |
| 15 | 96286_at | Cluster Incl AW060996 UI-M-BH1-amn-d-01-0-UI.s1 Mus musculus cDNA, 3 end/clone = UI-M-BH1-amn-d-01-0-UI/ | 629.9 | P | 485 | P | 476.6 | P | 592.1 |
| | | clone_end = 3/gb = AW060996/gi = 6008747/ug = Mm 29877/len = 542/STRA = for | | | | | | | |
| 15 | 97483_at | Cluster Incl M62952: Mus musculus ribosomal protein L19, complete cds/cds = (0,590)/gb = M62952/gi = 198642/ | 3119 | P | 2311.2 | A | 2667.6 | P | 2750.4 |
| | | ug = Mm 30806/len = 673/STRA = for | | | | | | | |
| 15 | 99654_s_at | Cluster Incl AA726579 vu93a07 r1 Mus musculus cDNA, 5 end/clone = IMAGE-1209684/clone_end = 5/gb = AA726579/ | 3474.8 | P | 2891.7 | P | 3091.4 | P | 3284.8 |
| | | gi = 2744286/ug = Mm 43751/len = 427/STRA = for | | | | | | | |
| 15 | 102425_at | Cluster Incl U61362 Mus musculus groucho-related gene 1 protein (Grg1) mRNA, complete cds/cds = (54,2366)/ | 57.6 | P | 103.5 | P | 89.1 | A | 114.9 |
| | | gb = U61362/gi = 1420928/ug = Mm 12823/len = 2381/STRA = for | | | | | | | |
| 15 | 94429_at | Cluster Incl L26479 Eukaryotic translation elongation factor 1 alpha 2/cds = (133,1524)/gb = L26479/gi = 1220409/ | 1091.7 | P | 824.6 | P | 974.3 | P | 1009.8 |
| | | ug = Mm 2645/len = 1782/STRA = for | | | | | | | |
| 15 | 98028_at | Cluster Incl M63849 Twist gene homolog, (Drosophila)/cds = (166,786)/gb = M63649/gi = 202243/ug = Mm 3280/len = 1485/ | 134.3 | P | 122.7 | P | 81.4 | P | 60.2 |
| | | STRA = for | | | | | | | |
| 15 | 101363_at | Cluster Incl AI644801 vb18e06 y1 Mus musculus cDNA, 5 end/clone = IMAGE-749314/clone_end = 5/gb = AI644801/ | 809.8 | P | 746.6 | P | 763.2 | P | 730.6 |
| | | gi = 4723276/ug = Mm 68974/len = 281/STRA = for | | | | | | | |
| 15 | 103680_at | Cluster Incl AA683850: vr05102 r1 Mus musculus cDNA, 5 end/clone = IMAGE-1110939/clone_end = 5/gb = AA683850/ | 210.2 | P | 46.4 | P | 74.4 | A | 262.5 |
| | | gi = 2670436/ug = Mm 23884/len = 535/STRA = for | | | | | | | |
| 15 | 103926_at | Cluster Incl AV380793: AV380793 Mus musculus cDNA, 3 end/clone = 9230102C24/clone_end = 3/gb = AV380793/ | 267.1 | P | 96.5 | A | 130.5 | P | 227.9 |
| | | gi = 6429870/ug = Mm.25082/len = 255/STRA = rev | | | | | | | |
| 15 | 92388_at | Cluster Incl AW045528 UI-M-BH1-aks-c-03-0-UI s1 Mus musculus cDNA, 3 end/clone = UI-M-BH1-aks-c-03-0-UI/ | 574.9 | P | 685.8 | P | 613.7 | P | 670.7 |
| | | clone_end = 3/gb = AW045528/gi = 5906057/ug = Mm 4055/len = 389/STRA = for | | | | | | | |
| 15 | 92527_at | Cluster Incl Z50190 Adenylate cyclase 9/cds = (42,4103)/gb = Z50190/gi = 1729772/ug = Mm 4294/len = 4457/STRA = for | 251.8 | P | 240.2 | P | 204.5 | P | 235.9 |
| 15 | 92927_at | Cluster Incl L10426 Mus musculus ets-related protein 81 (ER81) mRNA, complete cds/cds = (200,1633)/gb = L10426/ | 65.7 | P | 67 | P | 57.3 | A | 137.2 |
| | | gi = 193195/ug = Mm 4866/len = 2499/STRA = rev | | | | | | | |
| 15 | 93459_s_at | Cluster Incl AW122897: UI-M-BH2.1-apa-d-11-0-UI s1 Mus musculus cDNA, 3 end/clone = UI-M-BH2 1-apa-d-11-0-UI/ | 610.7 | P | 430.5 | P | 622.6 | A | 670.3 |
| | | clone_end = 3/gb = AW122897/gi = 6098427/ug = Mm 68712/len = 409/STRA = for | | | | | | | |
| 15 | 93964_s_at | Cluster Incl AF038995 Mus musculus putative RNA helicase RCK mRNA, partial cds/cds = (0,1359)/gb = AF038995/ | 325.7 | P | 218.2 | A | 419 | P | 407.1 |
| | | gi = 2745893/ug = Mm 97095/len = 1360/STRA = for | | | | | | | |
| 15 | 94766_at | Cluster Incl M17878 Mouse eEF-Tu gene encoding elongation factor Tu, 5 end/cds = (80,404)/gb = M17878/gi = 192991/ | 3192.9 | P | 2480.3 | P | 2669.1 | P | 2858.9 |
| | | ug = Mm.16317/len = 405/STRA = for | | | | | | | |
| 15 | 95883_at | Cluster Incl AW048355: UI-M-BH1-akk-f-02-0-UI s1 Mus musculus cDNA, 3 end/clone = UI-M-BH1-akk-f-02-0-UI/ | 174.5 | P | 174 | P | 174.7 | A | 190 |
| | | clone_end = 3/gb = AW048355/gi = 5908884/ug = Mm 23966/len = 483/STRA = rev | | | | | | | |
| 15 | 95964_at | Cluster Incl C77404 C77404 Mus musculus cDNA, 3 end/clone = J0030E06/clone_end = 3/gb = C77404/gi = 2517734/ | 135.6 | P | 130.7 | P | 122.1 | P | 191.5 |
| | | ug = Mm 24954/len = 508/STRA = rev | | | | | | | |
| 15 | 96493_at | Cluster Incl AA682091 vu13g02 s1 Mus musculus cDNA, 3 end/clone = IMAGE-1180562/clone_end = 3/gb = AA682091/ | 703.3 | P | 703.1 | P | 587.8 | P | 687.1 |
| | | gi = 2663123/ug = Mm 25192/len = 423/STRA = rev | | | | | | | |
| 15 | 99888_at | Cluster Incl U83190 Zonadhesin/cds = (0,1541)/gb = U83190/gi = 1932795/ug = Mm 7984/len = 1619/STRA = for | 448.5 | P | 425.3 | P | 393.8 | A | 430.7 |
| 15 | 100775_at | Cluster Incl C81070: C81070 Mus musculus cDNA, 3 end/clone = J0093A10/clone_end = 3/gb = C81070/gi = 2521400/ | 399.6 | P | 502.3 | P | 400.6 | P | 381.9 |
| | | ug = Mm.98626/len = 579/STRA = rev | | | | | | | |
| 15 | 99778_at | Cluster Incl AU020239: AU020239 Mus musculus cDNA, 3 end/clone = J0529F06/clone_end = 3/gb = AU020239/ | 2355.4 | P | 1938.8 | P | 2254 | P | 1383.3 |
| | | gi = 3375823/ug = Mm 97090/len = 434/STRA = rev | | | | | | | |
| 15 | 101213_at | Cluster Incl X15267 Mouse mRNA for acidic ribosomal phosophoprotein PO/cds = (73,1026)/gb = X15267/gi = 50025/ | 3025.3 | P | 1743.2 | A | 2200.8 | P | 2608.6 |
| | | ug = Mm 5286/len = 1094/STRA = for | | | | | | | |
| 15 | AFFX- | X00686 Mouse gene for 18S rRNA (_5, _M, _3 represent transcript regions 5 prime, Middle, | 104.7 | P | 53.2 | A | 50.3 | A | 190 |
| | 18SRNAMur/ | and 3 prime respectively) | | | | | | | |
| | X00686_5_at | | | | | | | | |
| 15 | 93520_at | Cluster Incl AF062655 Mus musculus plenty-of-prolines-101 mRNA, complete cds/cds = (23,2716)/gb = AF062655/ | 296.4 | P | 247.2 | A | 285.9 | P | 236.2 |
| | | gi = 3153820/ug = Mm 1963/len = 2957/STRA = for | | | | | | | |
| 15 | 97895_f_at | Cluster Incl AW125218: UI-M-BH2 1-aps-c-06-0-UI.s1 Mus musculus cDNA, 3 end/clone = UI-M-BH2.1-aps-c-06-0-UI/ | 1222.7 | P | 1045.4 | P | 1045.1 | P | 1173.9 |
| | | clone_end = 3/gb = AW125218/gi = 6100748/ug = Mm 28421/len = 319/STRA = for | | | | | | | |
| 15 | 94557_at | Cluster Incl AI850893 UI-M-BH0-ajt-g-07-0-UI.s1 Mus musculus cDNA, 3 end/clone = UI-M-BH0-ajt-g-07-0-UI/clone_end = 3/ | 381.4 | P | 382.1 | P | 284.9 | P | 200.7 |
| | | gb = AI850893/gi = 5494799/ug = Mm 29105/len = 375/STRA = for | | | | | | | |
| 15 | 96311_at | Cluster Incl M11533 Mouse myelin basic protein gene encoding 14 and 18.5 kd proteins/cds = (0,386)/gb = M11533/ | 153.5 | P | −39.8 | A | 100.4 | P | 161.1 |
| | | gi = 199045/ug = Mm 2992/len = 387/STRA = for | | | | | | | |
| 15 | 98623_g_at | Cluster Incl X71922 Insulin-like growth factor 2/cds = (0,236)/gb = X71922/gi = 393424/ug = Mm 3862/len = 3301/STRA = for | 2397.8 | P | 2130.5 | P | 2181.3 | P | 2126 |
| 15 | 99648_at | Cluster Incl X62772 Apolipoprotein A-II/cds = (47,355)/gb = X62772/gi = 50016/ug = Mm.43677/len = 473/STRA = for | 87.9 | P | 0.6 | M | 177.4 | P | 31.7 |
| 15 | 102854_s_at | Cluster Incl U03434: AT Pase, Cu++ transporting, alpha polypeptide/cds = (82,4557)/gb = U03434/gi = 451486/ug = Mm 14926/ | 123.5 | P | 37.1 | A | 66.1 | A | 113.1 |
| | | len = 4889/STRA = for | | | | | | | |
| 15 | 103318_at | Cluster Incl M74516 GA repeat binding protein, beta 1/cds = (132,1280)/gb = M74516/gi = 193384/ug = Mm 1764/len = 2598/ | 216.2 | P | 341.7 | P | 273.3 | P | 244 |
| | | STRA = for | | | | | | | |
| 15 | 103391_at | Cluster Incl AI988033: um10h12 x1 Mus musculus cDNA, 3 end/clone = IMAGE-2182727/clone_end = 3/gb = AI988033/ | 86.8 | P | 88.4 | P | 45.5 | P | 115.9 |
| | | gi = 5819827/ug = Mm 18654/len = 224/STRA = for | | | | | | | |
| 15 | 94326_r_at | Cluster Incl AW060921 UI-M-BH1-anv-c-12-0-UI s1 Mus musculus cDNA, 3 end/clone = UI-M-BH1-anv-c-12-0-UI/ | 552.3 | P | 552.1 | P | 434.2 | M | 504.2 |
| | | clone_end = 3/gb = AW060921/gi = 6008672/ug = Mm 24875/len = 438/STRA = for | | | | | | | |
| 15 | 97996_at | Cluster Incl U58134 Mus musculus poly(A) polymerase VI mRNA, complete cds/cds = (149,1030)/gb = U58134/gi = 1377869/ | 37.4 | P | 54 | P | 34.8 | P | 41.8 |
| | | ug = Mm 3170/len = 1046/STRA = for | | | | | | | |
| 15 | 100409_at | Cluster Incl X06340.Cadherin 3/cds = (45,2513)/gb = X06340/gi = 50267/ug = Mm 4658/len = 3187/STRA = for | 554.5 | P | 335.8 | P | 731.6 | P | 459 |
| 15 | 100879_at | Cluster Incl AF093775 Mus musculus alpha-actinin 3 (Actn3) mRNA, complete cds/cds = (30,2732)/gb = AF093775/ | 509.6 | A | 667.7 | P | 760.7 | P | 605.7 |
| | | gi = 3694989/ug = Mm 5316/len = 2855/STRA = for | | | | | | | |
| 15 | 101410_at | Cluster Incl AB000713 Mus musculus mCPE-R mRNA for CPE-receptor, complete cds/cds = (170,802)/gb = AB000713/ | 360.6 | P | 283.3 | P | 282.2 | P | 372.2 |
| | | gi = 2570126/ug = Mm 7339/len = 1783/STRA = for | | | | | | | |
| 15 | 92272_at | Cluster Incl AI060709: ub41h12 r1 Mus musculus cDNA, 5 end/clone = IMAGE-1380359/clone_end = 5/gb = AI060709/ | 93 | P | 102.8 | P | 97.2 | P | 83.6 |
| | | gi = 3336132/ug = Mm 3609/len = 421/STRA = for | | | | | | | |
| 15 | 92653_at | Cluster Incl AI482432.vg38e09 x1 Mus musculus cDNA, 3 end/clone = IMAGE-863656/clone_end = 3/gb = AI482432/ | 64.4 | P | 67.9 | A | 90.7 | P | 111.2 |
| | | gi = 4375658/ug = Mm 44063/len = 336/STRA = for | | | | | | | |
| 15 | 92756_r_at | Cluster Incl X73580 M musculus mRNA for secretin/cds = (8,409)/gb = X73580/gi = 313710/ug = Mm.4723/len = 490/ | 1597.3 | P | 1460.3 | P | 1455.2 | P | 1405.3 |
| | | STRA = for | | | | | | | |
| 15 | 93413_at | Cluster Incl AF003000 Telomeric repeat binding factor 2/cds = (65,1552)/gb = AF003000/gi = 2529441/ug = Mm 6402/ | 84.7 | A | 115 | P | 121.1 | P | 93.1 |
| | | len = 2119/STRA = for | | | | | | | |
| 15 | 93912_at | Cluster Incl AW047616 UI-M-BH1-akm-e-07-0-UI s1 Mus musculus cDNA, 3 end/clone = UI-M-BH1-akm-e-07-0-UI/ | 203.1 | P | 156 | A | 174.6 | P | 179.7 |
| | | clone_end = 3/gb = AW047616/gi = 5908145/ug = Mm.89571/len = 422/STRA = for | | | | | | | |
| 15 | 94780_at | Cluster Incl AI987985 um10a01 x1 Mus musculus cDNA, 3 end/clone = IMAGE-2182632/clone_end = 3/gb = AI987985/ | 148.4 | P | 117.1 | P | 56.9 | A | 125.6 |
| | | gi = 5819779/ug = Mm 16802/len = 492/STRA = rev | | | | | | | |
| 15 | 99866_at | Cluster Incl AI642417 vt02g03 x1 Mus musculus cDNA, 3 end/clone = IMAGE-1161940/clone_end = 3/gb = AI642417/ | 121.2 | M | 130 | P | 62.1 | A | 146.7 |
| | | gi = 4720892/ug = Mm.7202/len = 320/STRA = for | | | | | | | |
| 15 | 101755_f_at | Cluster Incl AJ005568: Small proline-rich protein 2J/cds = (0,329)/gb = AJ005568/gi = 3093374/ug = Mm.57084/len = 330/ | 1341.9 | P | 1291.1 | A | 1317.7 | P | 1598.1 |
| | | STRA = for | | | | | | | |
| 15 | 101763_at | Cluster Incl AF065145 Mus musculus melatonin-related receptor (Gpr50) mRNA, complete cds/cds = (25,1800)/ | 931.1 | P | 842 | M | 1108.7 | P | 1101 |
| | | gb = AF065145/gi = 3211764/ug = Mm 57095/len = 1801/STRA = for | | | | | | | |
| 15 | 96416_f_at | Cluster Incl Z30939 Histone gene complex 1/cds = (0,176)/gb = Z30939/gi = 496636/ug = Mm 88398/len = 287/STRA = for | 1449.4 | P | 944 | P | 955.9 | A | 997.8 |
| 15 | 101631_at | Cluster Incl AF009414: Mus musculus SOX11 (Sox11) mRNA, complete cds/cds = (288,1475)/gb = AF009414/gi = 2581939/ | 158.2 | P | 180.5 | A | 121.6 | P | 175.6 |
| | | ug = Mm 6238/len = 2896/STRA = for | | | | | | | |
| 15 | 95555_at | Cluster Incl AF030169: Mus musculus UNC-119 (Unc-119) mRNA, complete cds/cds = (69,791)/gb = AF030169/gi = 4103939/ | 125.9 | P | 211.3 | P | 65.4 | M | 120.8 |
| | | ug = Mm 27744/len = 1196/STRA = for | | | | | | | |
| 15 | 96779_f_at | Cluster Incl AI846315 UI-M-AK1-aez-h-08-0-UI.s1 Mus musculus cDNA, 3 end/clone = UI-M-AK1-aez-h-08-0-UI/ | 250.8 | P | 200 | A | 199 | M | 251.1 |
| | | clone_end = 3/gb = AI846315/gi = 5490221/ug = Mm 29063/len = 345/STRA = for | | | | | | | |
| 15 | 97924_at | Cluster Incl AJ132236 Mus musculus mRNA for UDP-N-acetylglucosamine 2-epimerase/N-acytmannosamine kinase/ | 331.2 | P | 297.7 | A | 396 | P | 295.2 |
| | | cds = (0,2168)/gb = AJ132236/gi = 4456672/ug = Mm 30193/len = 2169/STRA = for | | | | | | | |
| 15 | 100404_at | Cluster Incl L31959: Transgene insert site 737, Insertional mutation, polycystic kidney disease/cds = (71,2545)/gb = L31959/ | 533.3 | A | 720.6 | P | 524 | P | 505 |
| | | gi = 499646/ug = Mm.4653/len = 3024/STRA = for | | | | | | | |
| 15 | 100923_at | Cluster Incl AJ249706 Mus musculus partial mRNA for myosin X (myo 10 gene)/cds = (0,6092)/gb = AJ249706/gi = 5921504/ | 348.4 | P | 277.9 | A | 314.8 | P | 346.5 |
| | | ug = Mm 60590/len = 6093/STRA = for | | | | | | | |
| 15 | 101855_at | Cluster Incl Y14754 Microtubule-associated protein 6/cds = (0,2720)/gb = Y14754/gi = 2769586/ug = Mm 86654/len = 2721/ | 35.8 | P | 35.1 | A | 34.3 | P | 60.4 |
| | | STRA = rev | | | | | | | |
| 15 | 103701_at | Cluster Incl AA420331.vc51c06 r1 Mus musculus cDNA, 3 end/clone = IMAGE-778090/clone_end = 3/gb = AA420331/ | 185.1 | P | 72 | P | 138.6 | P | 167.4 |
| | | gi = 2080888/ug = Mm.24035/len 332/STRA = for | | | | | | | |
| 15 | 103779_at | Cluster Incl AI852829: UI-M-BH0-ajk-b-01-0-UI s1 Mus musculus cDNA, 3 end/clone = UI-M-BH0-ajk-b-01-0-UI/ | 498.9 | P | 443.7 | P | 409.2 | P | 316.1 |
| | | clone_end = 3/gb = AI852829/gi = 5496735/ug = Mm 24862/len = 366/STRA = for | | | | | | | |
| 15 | 104439_at | Cluster Incl AI840198: UI-M-AO0-acc-h-05-o-Ul.s1 Mus musculus cDNA, 3 end/clone = UI-M-AO0-acc-h-05-0-UI/ | 121.7 | P | 119.6 | P | 103 | M | 67.1 |
| | | clone_end = 3/gb = AI840198/gi = 5474411/ug = Mm.29271/len = 283/STRA = rev | | | | | | | |
| 15 | 104442_at | Cluster Incl AI605538: vo36b03.x1 Mus musculus cDNA 3 end/clone = IMAGE-1051949/clone_end = 3/gb = AI605538/ | 246.4 | P | 96.6 | A | 89.6 | A | 221.4 |
| | | gi = 4614705/ug = Mm 29302/len = 286/STRA = rev | | | | | | | |
| 15 | 92399_at | Cluster Incl D26532 Core binding factor alpha 2/cds = (1573,2736)/gb = D26532/gi = 493683/ug = Mm 4081/len = 4295/ | 84.1 | P | 52.4 | A | 40 | A | 100.9 |
| | | STRA = for | | | | | | | |
| 15 | 92475_g_at | Cluster Incl AF083497 untitled/cds = (31,3255)/gb = AF083497/gi = 3986469/ug = Mm 42128/len = 3924/STRA = for | 56.7 | P | 102.6 | P | 21.9 | P | 23.8 |
| 15 | 93464_at | Cluster Incl AI581567 vw82e12.x1 Mus musculus cDNA, 3 end/clone = lMAGE-1261486/clone_end = 3/gb = AI561567/ | 151.5 | P | 55.6 | A | 10.1 | A | 82.4 |
| | | gi = 4512912/ug = Mm 6910/len = 256/STRA = for | | | | | | | |
| 15 | 93466_at | Cluster Incl AF022962 Secretory protein SEC8 (S cerevisiae)/cds = (29,2944)/gb = AF022962/gi = 2547135/ug = Mm 6925/ | 143.7 | P | 53.9 | M | 91.5 | P | 127.2 |
| | | len = 3155/STRA = for | | | | | | | |
| 15 | 95938_at | Cluster Incl AA612212 vo04a01.r1 Mus musculus cDNA, 5 end/clone = IMAGE-1040424/clone_end = 5/gb = AA612212/ | 69.5 | P | 61.2 | P | 85.6 | A | 63.9 |
| | | gi = 2460890/ug = Mm.24840/len = 357/STRA = for | | | | | | | |
| 15 | 96513_at | Cluster Incl AA794350 vr46a11 s1 Mus musculus cDNA, 5 end/clone = IMAGE-1123676/clone_end = 5/gb = AA794350/ | 129 | P | 113.8 | M | 135.1 | A | 200.6 |
| | | gi = 2857305/ug = Mm.25583/len = 109/STRA = rev | | | | | | | |
| 15 | 97744_at | Cluster Incl AI847619 UI-M-AP1-agg-d-12-0-UI s1 Mus musculus cDNA, 3 end/clone = UI-M-AP1-agg-d-12-0-UI/ | 442.1 | A | 396.3 | A | 412.6 | P | 384.3 |
| | | clone_end = 3/gb = AI847619/gi = 5491525/ug = Mm 40174/len = 364/STRA = rev | | | | | | | |
| 15 | 102152_f_at | Cluster Incl M16724 Mouse Ig germline H-chain gene V-region (subfamily T15), exons 1 and 2/cds = (0,187)/gb = M16724/ | 499.1 | P | 307.1 | P | 378.3 | A | 426.4 |
| | | gi = 196262/ug = Mm 46800/len = 188/STRA = for | | | | | | | |
| 15 | AFFX- | X00686 Mouse gene for 18S rRNA (_5, _M, _3 represent transcript regions 5 prime, Middle, | 137 | M | 431.3 | P | 219.3 | M | 342.4 |
| | 18SRNAMur/ | and 3 prime respectively) | | | | | | | |
| | X00686_3_at | | | | | | | | |
| 15 | 93087_r_at | Cluster lncl AA267185 mz92g02 r1 Mus musculus cDNA, 5 end/clone = IMAGE-720914/clone_end = 5/gb = AA267185/ | 377 | P | 241 | A | 275.5 | M | 329.6 |
| | | gi = 1903945/ug = Mm 16253/len = 229/STRA = for | | | | | | | |
| 15 | 95138_at | Cluster Incl AI836568 UI-M-AP0-abf-a-06-0-UI s1 Mus musculus cDNA, 3 end/clone = UI-M-AP0-abf-a-06-0-UI/ | 123.1 | A | 101.1 | A | 171.9 | P | 157.4 |
| | | clone_end = 3/gb = AI836568/gi = 5470781/ug = Mm 29432/len = 285/STRA = for | | | | | | | |
| 15 | 104109_at | Cluster Incl AI853773 UI-M-BH0-ajg-e-03-0-UI s1 Mus musculus cDNA, 3 end/clone = UI-M-BH0-ajg-e-03-0-UI/ | 152.7 | P | 90.5 | P | 131.7 | P | 124.4 |
| | | clone_end = 3/gb = AI853773/gi = 5497679/ug = Mm 21912/len = 453/STRA = for | | | | | | | |
| 15 | 104590_at | Cluster Incl L13171 Myocyte enhancer factor 2C/cds = UNKNOWN/gb = L13171/gi = 293728/ug = Mm 24001/len = 1656/ | 230.1 | P | 335.9 | A | 183.8 | A | 269.2 |
| | | STRA = for | | | | | | | |
| 15 | 97355_at | Cluster Incl AW050020 UI-M-BH1-amm-a-08-0-UI s1 Mus musculus cDNA, 3 end/clone = UI-M-BH1-amm-a-08-0-UI/ | 60.9 | P | 48.1 | M | 76.2 | P | 90 |
| | | clone_end = 3/gb = AW050020/gi = 5910549/ug = Mm 29632/len = 458/STRA = for | | | | | | | |
| 15 | 97975_at | Cluster Incl D85612 Nuclear factor of activated T-cells, cytoplasmic 3/cds = (7,3234)/gb = D85612/gi = 1906311/ | 332.7 | P | 167.9 | A | 161.1 | A | 287.8 |
| | | ug = Mm 3107/len = 3638/STRA = for | | | | | | | |
| 15 | 103460_at | Cluster Incl AI849939: UI-M-BG0-ahz-g-03-0-UI s1 Mus musculus cDNA, 3 end/clone = UI-M-BG0-ahz-g-03-0-UI/ | 198 | A | 176.5 | A | 312.8 | P | 302.3 |
| | | clone_end = 3/gb = AI849939/gi = 5493845/ug = Mm 21697/len = 440/STRA = for | | | | | | | |
| 15 | 103925_at | Cluster Incl AW120605 UI-M-BH2.3-anx-f-01-0-UI.s1 Mus musculus cDNA, 3 end/clone = UI-M-BH2 3-anx-f-01-0-UI/ | 232.8 | P | 121.3 | A | 165.2 | A | 277.7 |
| | | clone_end = 3/gb = AW120605/gi = 6095938/ug = Mm 25080/len = 151/STRA = for | | | | | | | |
| 15 | 94104_at | Cluster Incl AF051911 Telomerase reverse transcriptase/cds = (29,3397)/gb = AF051911/gi = 3005591/ug= Mm 10109/ | 225.4 | P | 173.8 | A | 230.5 | P | 239.5 |
| | | len = 3426/STRA = for | | | | | | | |
| 15 | 98303_at | Cluster Incl X79131: M. musculus mRNA for IB3/5-polypeptide/cds = (833,2869)/gb = X79131/gi = 485857/ug = Mm 42143/ | 387.2 | P | 339.2 | M | 281.6 | M | 227.6 |
| | | len = 3008/STRA = for | | | | | | | |
| 15 | 99439_at | Cluster Incl X67735.MAS1 oncogene/cds = (0,974)/gb = X67735/gi = 53011/ug = Mm 57182/len = 975/STRA = for | 257 | M | 296.3 | M | 263.3 | P | 359.1 |
| 15 | 95869_at | Cluster Incl C78041 C78041 Mus musculus cDNA, 3 end/clone = J0041H05/clone_end = 3/gb = C78041/gi = 2518371/ | 25.5 | A | 29.5 | A | 10.1 | A | 48.3 |
| | | ug = Mm.24980/len = 538/STRA = rev | | | | | | | |
| 15 | 95879_at | Cluster Incl C79052 C79052 Mus musculus cDNA, 3 end/clone = J0059F07/clone_end = 3/gb = C79052/gi = 2519382/ | 28.8 | P | −4.3 | A | −10 | A | 25.5 |
| | | ug = Mm 25008/len = 608/STRA = rev | | | | | | | |
| 15 | 100686_at | Cluster Incl M20632 Mouse LLRep3 protein mRNA from a repetitive element, complete cds/cds = (1022,1687)/gb = M20632/ | 2619.8 | P | 1731 | A | 2064.3 | A | 2178.7 |
| | | gi = 200811/ug = Mm 1129/len = 1728/STRA = for | | | | | | | |
| 15 | AFFX- | X00686 Mouse gene for 18S rRNA (_5, _M, _3 represent transcript regions 5 prime, Middle, | 106.8 | A | −43.2 | A | −11.5 | A | 45.7 |
| | 18SRNAMur/ | and 3 prime respectively) | | | | | | | |
| | X00686_M_at | | | | | | | | |
| 15 | 99980_at | Cluster Incl M35986 Mouse homeobox-6 1 protein (Hox-6.1) mRNA, 3 end/cds = (0,317)/gb = M35986/gi = 193981/ | 114.1 | A | 103.9 | A | 107.7 | M | 141.8 |
| | | ug = Mm 4444/len = 318/STRA = for | | | | | | | |
| 15 | 102695_at | Cluster Incl AF021335 Mus musculus T cell receptor gamma locus, TCR gamma 2 and gamma 4 gene clusters/ | 254.3 | P | 254.5 | P | 241.5 | A | 286.3 |
| | | cds = (0,571)/gb = AF021335/gi = 2444469/ug = Mm 14145/len = 572/STRA = for | | | | | | | |
| 15 | 102865_at | Cluster Incl U58993 MAD homolog 5 (Drosophila)/cds = (266,1663)/gb = U58993/gi = 1518646/ug = Mm.1470/len = 2185/ | 121.4 | A | 73.1 | A | 90.8 | A | 101 |
| | | STRA= for | | | | | | | |
| 15 | 102871_at | Cluster Incl L77867 Mus musculus MEP mRNA, complete cds/cds = (202,3246)/gb = L77867/gi = 1911182/ug = Mm.1480/ | −35.7 | A | 50.8 | P | 154.7 | P | 254.4 |
| | | len = 3464/STRA = for | | | | | | | |
| 15 | 103229_at | Cluster Incl U64828 Mus musculus steroid receptor coactivator-1 (mSRC-1) mRNA, complete cds/cds = (625,4842)/ | 148.9 | A | 126.6 | A | 158.1 | P | 139.1 |
| | | gb = U64828/gi = 1490875/ug = Mm 2028/1en = 5169/STRA = for | | | | | | | |
| 15 | 104010_at | Cluster Incl U62906: Mus musculus zinc finger protein 94 (Zfp94) mRNA, complete cds/cds = (180,1742)/gb = U62906/ | 154.4 | A | 105.5 | A | 88.4 | A | 136.6 |
| | | gi = 4097496/ug = Mm.26782/len = 1997/STRA = for | | | | | | | |
| 15 | 92364_at | Cluster Incl AB028499 Mus musculus mRNA for Flamingo 1, complete cds/cds = (0,8762)/gb = AB028499/gi = 5832710/ | 510 | A | 738 | A | 717.8 | A | 589.5 |
| | | ug = Mm 39728/len = 8950/STRA = for | | | | | | | |
| 15 | 93900_at | Cluster Incl AW050268 UI-M-BH1-ans-d-03-0-UI.s1 Mus musculus cDNA, 3 end/clone = UI-M-BH1-ans-d-03-0-UI/ | 768.8 | P | 646.6 | A | 422.7 | A | 532.1 |
| | | clone_end = 3/gb = AW050268/gi = 5910797/ug = Mm 89316/len = 221/STRA = for | | | | | | | |
| 15 | 95284_at | Cluster Incl AI662509 mt33c08 x1 Mus musculus cDNA, 3 end/clone = IMAGE-822862/clone_end = 3/gb = AI662509/ | 56.3 | A | 105 | P | 79.3 | P | 65 |
| | | gi = 4766092/ug = Mm.1850/len = 231/STRA = for | | | | | | | |
| 15 | 95995_at | Cluster Incl C80108 C80108 Mus musculus cDNA, 3 end/clone = J0076F01/clone_end = 3/gb = C80108/gi = 2520438/ | 118.6 | P | 91.5 | A | 71.3 | A | 102.9 |
| | | ug = Mm 25061/1en = 349/STRA = rev | | | | | | | |
| 15 | 100282_at | Cluster Incl Z23107 5-hydroxytryptamine (serotonin) receptor 7/cds = (108,1454)/gb = Z23107/gi = 396586/ug = Mm 8053/ | 196.4 | M | 397.1 | A | 317 | P | 275.2 |
| | | len = 1540/STRA = for | | | | | | | |
| 15 | 95872_at | Cluster Incl C78535: C78535 Mus musculus cDNA, 3 end/clone = J0050H08/clone_end = 3/gb = C78535/gi = 2518865/ | 53.3 | M | 102.3 | P | 113.3 | P | 85.8 |
| | | ug= Mm.24993/len = 576/STRA = rev | | | | | | | |
| 15 | 101159_at | Cluster Incl Z23066: Microphthalmia-associated transcription factor/cds = (129,1388)/gb = Z23066/gi = 396739/ug = Mm 4978/ | 128.4 | M | 23.8 | A | 25.6 | A | 75.5 |
| | | len = 1922/STRA = for | | | | | | | |
| 15 | 101618_r_at | Cluster Incl U59758 Mus musculus p53-variant (p53) mRNA, partial cds/cds = (0,161)/gb= U59758/gi = 1399879/ | 443 | A | 362.1 | P | 392 | A | 385.7 |
| | | ug = Mm.6186/len = 846/STRA = for | | | | | | | |
| 15 | 103348_at | Cluster Incl AI837688 UI-M-AK0-adj-c-07-0-UI.s1 Mus musculus cDNA, 3 end/clone = UI-M-AK0-adj-c-07-0-UI/ | 367.5 | P | 171 | A | 84.9 | M | 380.5 |
| | | clone_end = 3/gb = AI837688/gi = 5471901/ug = Mm 18311/len = 392/STRA = for | | | | | | | |
| 15 | 98507_at | Cluster Incl AI834950 UI-M-AM1-afw-b-05-0-UI.s1 Mus musculus cDNA, 3 end/clone = UI-M-AM1-afw-b-05-0-UI/ | 205.1 | P | 166.3 | A | 162.9 | A | 176.1 |
| | | clone_end = 3/gb = AI834950/gi = 5469163/ug = Mm 35822/len = 315/STRA = rev | | | | | | | |
| 15 | 100021_at | Cluster Incl M17640 Mus musculus acetylcholine receptor alpha-subunit mRNA, complete cds/cds = (104,1477)/ | 194.8 | A | −39 | P | 43.2 | P | 67.2 |
| | | gb = M17640/gi = 2073542/ug = Mm 4583/len = 1860/STRA = for | | | | | | | |
| 15 | 101381_at | Cluster Incl D86066 Mus musculus mRNA for mouse rabaptin-5, complete cds/cds = (50,2638)/gb = D86066/gi = 2329848/ | 57.3 | P | 62.2 | P | 45 | A | 35.2 |
| | | ug = Mm 7087/len = 3095/STRA = for | | | | | | | |
| 15 | 104195_at | Cluster Incl AA939440: vz49d01.r1 Mus musculus cDNA, 5 end/clone = IMAGE-1329793/clone_end = 5/gb = AA939440/ | 236.1 | A | 207.9 | M | 147 | A | 188.6 |
| | | gi = 3100217/ug = Mm 27503/len = 379/STRA = for | | | | | | | |
| 15 | 104415_at | Cluster Incl AA833293 ud05d05 r1 Mus musculus cDNA, 5 end/clone = IMAGE-1434249/clone_end = 5/gb= AA833293/ | 80.9 | A | 52.2 | A | 90.8 | A | 75 |
| | | gi = 2907021/ug = Mm 28907/len = 548/STRA = for | | | | | | | |
| 15 | 93155_at | Cluster Incl AI847069 UI-M-AP1-agn-c-04-0-UI s1 Mus musculus cDNA, 3 end/clone = UI-M-AP1-agn-c-04-0-UI/ | 28.6 | A | 43 | P | 34.2 | A | 42.7 |
| | | clone_end = 3/gb = AI847069/gi = 5490975/ug = Mm.5263/len = 263/STRA = for | | | | | | | |
| 15 | 97711_at | Cluster Incl AI606967 vw37a01 x1 Mus musculus cDNA, 3 end/clone = IMAGE-1245960/clone_end = 3/gb = AI606967/ | 69.9 | A | 85.2 | A | 60 | A | 52 |
| | | gi = 4616134/ug = Mm 38364/len = 387/STRA = rev | | | | | | | |
| 15 | 99908_at | Cluster Incl AB010557 Mus musculus mRNA for Pax4, complete cds/cds = (206,1255)/gb = AB010557/gi = 2809092/ | 443.3 | M | 350.3 | A | 428.3 | M | 468.5 |
| | | ug = Mm 8026/len = 1295/STRA = for | | | | | | | |
| 15 | 100749_at | Cluster Incl AA414976: vc49e02 r1 Mus musculus cDNA, 3 end/clone = IMAGE-777914/clone_end = 3/gb = AA414976/ | 125 | A | 153.8 | A | 208.1 | P | 162.4 |
| | | gi = 2075103/ug = Mm 3802/len = 332/STRA = rev | | | | | | | |
| 16 | 92567_at | Cluster Incl L02918 Mouse procollagen type V alpha 2 (Col5a-2) mRNA, complete cds/cds = (0,4493)/gb = L02918/ | 2065.5 | P | 2956.5 | P | 3397.9 | P | 1825.1 |
| | | gi = 309180/ug = Mm 10299/len = 4494/STRA = for | | | | | | | |
| 16 | 92587_at | Cluster Incl L29123 Mus musculus adrenodoxin mRNA, complete cds/cds = (88,654)/gb = L29123/gi = 457297/ug = Mm 1061/ | 623.2 | P | 607.2 | P | 670.1 | P | 554.3 |
| | | len = 883/STRA = for | | | | | | | |
| 16 | 92795_at | Cluster Incl M72414 Microtubule-associated protein 4/cds = (490,3867)/gb = M72414/gi = 456554/ug = Mm 12625/len = 6082/ | 367.7 | P | 532.6 | P | 466.6 | P | 347 |
| | | STRA = for | | | | | | | |
| 16 | 92816_r_at | Cluster Incl X03039.Eukaryotic translation Initiation factor 4A1/cds = UNKNOWN/gb = X03039/gi = 50812/ug = Mm 12858/ | 2099.8 | P | 2538.8 | P | 2255.4 | P | 2347.6 |
| | | len = 1407/STRA = for | | | | | | | |
| 16 | 92833_at | Cluster Incl L07645 Histidine ammonia tyase/cds = UNKNOWN/gb= L07645/gi = 193751/ug = Mm 13000/len = 2281/ | 1780.5 | P | 2199.5 | P | 2382.7 | P | 1603.2 |
| | | STRA = for | | | | | | | |
| 16 | 93029_at | Cluster Incl U68564 Isocitrale dehydrogenase 3 (NAD+), gamma/cds = (42,1223)/gb = U68564/gi = 1546828/ug = Mm 14825/ | 933.8 | P | 1058.2 | P | 1079.5 | P | 955 |
| | | len = 1335/STRA = for | | | | | | | |
| 16 | 93316_at | Cluster Incl AB017026 Mus musculus mRNA for oxysterol-binding protein, complete cds/cds = (827,2059)/gb = AB017026/ | 453.1 | P | 529.6 | P | 585.4 | P | 288.4 |
| | | gi = 3551522/ug = Mm 18502/len = 2931/STRA = for | | | | | | | |
| 16 | 93333_at | Cluster Incl U05333 Mus musculus co-chaperonin cofactor A mRNA, complete cds/cds = (7,333)/gb = U05333/gi = 1117784/ | 562.5 | P | 548.5 | P | 484 | P | 474 |
| | | ug = Mm 1863/len = 508/STRA = for | | | | | | | |
| 16 | 93571_at | Cluster Incl M74773 Beta-spectrin 2, non-erythrocytic/cds = (366,7457)/gb = M74773/gi = 598330/ug = Mm 20405/len = 7524/ | 1807 | P | 1932.5 | P | 1835.1 | P | 1488.3 |
| | | STRA = for | | | | | | | |
| 16 | 93574_at | Cluster Incl AF036164 Mus musculus pigment epithelium-derived factor (PEDF) mRNA, complete cds/cds = (33,1286)/ | 310.7 | P | 595.9 | P | 714.7 | P | 466.6 |
| | | gb = AF036164/gi = 3808220/ug = Mm 2044/len = 1648/STRA = for | | | | | | | |
| 16 | 93731_at | Cluster Incl AF090334 Mus musculus FK506 binding protein (Fkbp63) mRNA, complete cds/cds = (74,1786)/gb = AF090334/ | 974.6 | P | 1434.8 | P | 1418.1 | P | 992.8 |
| | | gi = 3860028/ug = Mm 20943/len = 2945/STRA = for | | | | | | | |
| 16 | 93750_at | Cluster Incl J04953 Gelsolin/cds = (18,2213)/gb = J04953/gi = 193463/ug = Mm 21109/len = 2438/STRA = for | 1737.3 | P | 2430.9 | P | 2831.8 | P | 1856.4 |
| 16 | 93754_at | Cluster Incl AF030343 Mus musculus peroxisomal/mitochondrial dienoyl-CoA isomerase ECH1p (Ech1) mRNA, complete | 834.4 | P | 1087 | P | 1034.9 | P | 661.1 |
| | | cds/cds = (23,1006)/gb = AF030343/gi = 2606085/ug = Mm 2112/len = 1138/STRA = for | | | | | | | |
| 16 | 94031_at | Cluster Incl X95403 M musculus mRNA for GTP-binding protein/cds = (207,845)/gb = X95403/gi = 1177546/ug = Mm.21905/ | 1145.6 | P | 1322.5 | P | 1226.3 | P | 943.9 |
| | | len = 1008/STRA = for | | | | | | | |
| 16 | 94059_at | Cluster Incl AI852524 UI-M-BH0-aiw-d-04-0-UI.s1 Mus musculus cDNA, 3 end/clone = UI-M-BH0-aiw-d-04-0-UI/ | 873 | P | 1258.3 | P | 895 | P | 877.8 |
| | | clone_end = 3/gb = AI852524/gi = 5496430/ug = Mm 22029/len = 382/STRA = for | | | | | | | |
| 16 | 94233_at | Cluster Incl AW048642 UI-M-BH1-amd-d-06-0-UI s1 Mus musculus cDNA, 3 end/clone = UI-M-BH1-amd-d-06-0-UI/ | 243.5 | P | 290.5 | P | 277.1 | P | 186.7 |
| | | clone_end = 3/gb = AW048642/gi = 5909171/ug = Mm.22294/len = 480/STRA = for | | | | | | | |
| 16 | 94269_at | Cluster Incl L40934 Mus musculus rab6/rab5-associated protein (rab6) mRNA, partial cds/cds = (0,307)/gb = L40934/ | 2010.5 | P | 2429.1 | P | 2502.9 | P | 2409.6 |
| | | gi = 722666/ug = Mm 22473/len = 308/STRA = for | | | | | | | |
| 16 | 94284_at | Cluster Incl AW122731 UI-M-BH2 2-aot-e-08-0-UI s1 Mus musculus cDNA, 3 end/clone = UI-M-BH2 2-aot-e-08-0-UI/ | 824 | P | 1035.9 | P | 836.6 | P | 732.5 |
| | | clone_end = 3/gb = AW122731/gi = 6098261/ug = Mm 22560/len = 204/STRA = for | | | | | | | |
| 16 | 94289_r_at | Cluster Incl AI851574 UI-M-BH0-aki-f-10-0-UI s1 Mus musculus cDNA, 3 end/clone = UI-M-BH0-aki-f-10-0-UI/clone_end = 3/ | 691.9 | P | 1012.4 | P | 817 | P | 508 |
| | | gb = AI851574/gi = 5495480/ug = Mm 22575/len = 459/STRA = for | | | | | | | |
| 16 | 94870_f_at | Cluster Incl AW124226 UI-M-BH2 1-aph-d-10-0-UI.s1 Mus musculus cDNA, 3 end/clone = UI-M-BH2.1-aph-d-10-0-UI/ | 304.6 | P | 328.3 | P | 257.3 | P | 285.1 |
| | | clone_end = 3/gb = AW124226/gi = 6099756/ug = Mm 23755/len = 382/STRA = for | | | | | | | |
| 16 | 94872_at | Cluster Incl Y08135 M musculus mRNA for ASM-like phosphodiesterase 3a/cds = (92,1429)/gb = Y08135/gi = 1552349/ | 362.3 | P | 307.9 | P | 358.7 | P | 223.2 |
| | | ug = Mm 2379/len = 1758/STRA = for | | | | | | | |
| 16 | 94878_at | Cluster Incl AW125224 UI-M-BH2 1-aps-d-02-0-UI s1 Mus musculus cDNA, 3 end/clone = UI-M-BH2 1-aps-d-02-0-UI/ | 548.4 | P | 786.6 | P | 592.4 | P | 509.6 |
| | | clone_end = 3/gb = AW125224/gi = 6100754/ug = Mm 23839/len = 356/STRA = for | | | | | | | |
| 16 | 94889_at | Cluster Incl AW121235 UI-M-BH2 3-aok-f-05-0-UI s1 Mus musculus cDNA, 3 end/clone = UI-M-BH2 3-aok-f-05-0-UI/ | 1900.3 | P | 2041.3 | P | 1987.9 | P | 1780.8 |
| | | clone_end = 3/gb = AW121235/gi = 6096568/ug = Mm 23943/len = 293/STRA = for | | | | | | | |
| 16 | 94892_r_at | Cluster Incl M27938: Male enhanced antigen 1/cds = (80,604)/gb = M27938/gi = 199115/ug = Mm.2395/len = 841/STRA = for | 1141.6 | P | 1319 | P | 998 | P | 891.8 |
| 16 | 94899_at | Cluster Incl U73200. Mus musculus p116Rip mRNA, complete cds/cds = (191,3265)/gb = U73200/gi = 1657836/ug = Mm 2402/ | 1865.6 | P | 2284.4 | P | 2107.6 | P | 1828.3 |
| | | len = 4010/STRA = for | | | | | | | |
| 16 | 95411_at | Cluster Incl AB025407 Mus musculus mRNA for sid2057p, complete cds/cds = (83,628)/gb = AB025407/gi = 5931562/ | 1203.8 | P | 1450.2 | P | 1262.8 | P | 1197.4 |
| | | ug = Mm 24231/len = 1400/STRA = for | | | | | | | |
| 16 | 96049_at | Cluster Incl X53928: M musculus mRNA for PGI (biglycan)/cds = (234,1343)/gb = X53928/gi = 53666/ug = Mm 2608/ | 2619.8 | P | 3376.2 | P | 3376.6 | P | 2284.7 |
| | | len = 1964/STRA = for | | | | | | | |
| 16 | 96629_at | Cluster Incl X04097: Mouse kidney lestosterone-regulated RP2 mRNA/cds = (41,1114)/gb = X04097/gi = 54003/ | 333.7 | P | 433.4 | P | 365.1 | P | 376.4 |
| | | ug = Mm.27194/len = 2005/STRA = for | | | | | | | |
| 16 | 96634_at | Cluster Incl AI850090: UI-M-BG0-aia-g-01-0-UI.s1 Mus musculus cDNA, 3 end/clone = UI-M-BG0-aia-g-01-0-UI/ | 341 | P | 352.4 | P | 318 | P | 223.2 |
| | | clone_end = 3/gb = AI850090/gi = 5493996/ug = Mm 27227/len = 481/STRA = for | | | | | | | |
| 16 | 96641_at | Cluster Incl AW060674 UI-M-BH1-anl-d-04-0-UI s1 Mus musculus cDNA, 3 end/clone = UI-M-BH1-anl-d-04-0-UI/ | 476.9 | P | 660.4 | P | 603.3 | P | 485.1 |
| | | clone_end = 3/gb = AW060674/gi = 6008425/ug = Mm 27285/len = 486/STRA = for | | | | | | | |
| 16 | 96708_at | Cluster Incl AW120643 UI-M-BH2 3-anz-f-05-0-UI.s1 Mus musculus cDNA, 3 end/clone = UI-M-BH2 3-anz-f-05-0-UI/ | 1023.1 | P | 1213.2 | P | 1364.4 | P | 1023.5 |
| | | clone_end = 3/gb = AW120643/gi = 6096016/ug = Mm 27606/len = 505/STRA = for | | | | | | | |
| 16 | 97277_at | Cluster Incl AI844179 UI-M-AL1-ahk-a-04-0-UI s1 Mus musculus cDNA, 3 end/clone = UI-M-AL1-ahk-a-04-0-UI/ | 221.1 | P | 351.2 | P | 323.8 | P | 270.7 |
| | | clone_end = 3/gb = AI844179/gi = 5488085/ug = Mm 27951/len = 281/STRA = for | | | | | | | |
| 16 | 97841_at | Cluster Incl AI838663 UI-M-AO0-aca-d-07-0-UI.s1 Mus musculus cDNA, 3 end/clone = UI-M-AO0-aca-d-07-0-UI/ | 978.6 | P | 1259.8 | P | 1254.3 | P | 1120.8 |
| | | clone_end = 3/gb = AI838663/gi = 5472876/ug = Mm 28258/len = 368/STRA = for | | | | | | | |
| 16 | 97901_at | Cluster Incl X60831 Mouse ubf gene for transcription factor UBF/cds = UNKNOWN/gb = X60831/gi = 55115/ug = Mm 2845/ | 515.2 | P | 572.8 | P | 516.4 | P | 324 |
| | | len = 3031/STRA = for | | | | | | | |
| 16 | 94454_at | Cluster Incl AF085348 Mus musculus proline-nch protein (Bprp) mRNA, complete cds/cds = (15,521)/gb = AF085348/ | 926.1 | P | 1142.9 | P | 1180.5 | P | 1058.7 |
| | | gi = 3549610/ug = Mm 28797/len = 1779/STRA = for | | | | | | | |
| 16 | 94531_at | Cluster Incl AW124582: UI-M-BH2 1-apt-e-11-0-UI s1 Mus musculus cDNA, 3 end/clone = UI-M-BH2 1-apt-e-11-0-UI/ | 401.4 | P | 416.7 | P | 321.3 | P | 275.4 |
| | | clone_end = 3/gb = AW124582/gi = 6100112/ug = Mm 29042/len = 360/STRA = for | | | | | | | |
| 16 | 94549_at | Cluster Incl AI315650: uj31d03 x1 Mus musculus cDNA, 3 end/clone = IMAGE-1921541/clone_end = 3/gb = AI315650/ | 76.9 | P | 139.9 | P | 198.3 | P | 135.4 |
| | | gi = 4030917/ug = Mm 29078/len = 514/STRA = for | | | | | | | |
| 16 | 95054_at | Cluster Incl AI849620: UI-M-AH1-agr-h-02-0-UI s1 Mus musculus cDNA, 3 end/clone = UI-M-AH1-agr-h-02-0-UI/ | 498.6 | P | 499.9 | P | 493.6 | P | 496.1 |
| | | clone_end = 3/gb = AI849620/gi = 5493526/ug = Mm 29144/len = 340/STRA = for | | | | | | | |
| 16 | 95074_at | Cluster Incl AW125309: UI-M-BH2.1-apy-d-05-0-UI s1 Mus musculus cDNA, 3 end/clone = UI-M-BH2 1-apy-d-05-0-UI/ | 343.8 | P | 338.8 | P | 330.7 | P | 303.5 |
| | | clone_end = 3/gb = AW125309/gi = 6100839/ug = Mm 29198/len = 276/STRA = for | | | | | | | |
| 16 | 95136_at | Cluster Incl AW049185: UI-M-BH1-amq-g-01-0-UI.s1 Mus musculus cDNA, 3 end/clone = UI-M-BH1-amq-g-01-0-UI/ | 428.9 | P | 912.5 | P | 793.2 | P | 358.5 |
| | | clone_end = 3/gb = AW049185/gi = 5909714/ug = Mm 29430/len = 483/STRA = for | | | | | | | |
| 16 | 95701_at | Cluster Incl AW124069: UI-M-BH2.1-apn-f-11-0-UI.s1 Mus musculus cDNA, 3 end/clone = UI-M-BH2 1-apn-f-11-0-UI/ | 212.3 | P | 252.2 | P | 244.9 | P | 251.8 |
| | | clone_end = 3/gb = AW124069/gi = 6099599/ug = Mm 29690/len = 390/STRA = for | | | | | | | |
| 16 | 95732_at | Cluster Incl AW047746: UI-M-BH1-alk-b-11-0-UI s1 Mus musculus cDNA, 3 end/clone = UI-M-BH1-alk-b-11-0-UI/ | 602.8 | P | 1080.9 | P | 664.8 | P | 522.8 |
| | | clone_end = 3/gb = AW047746/gi = 5908275/ug = Mm 29743/len = 406/STRA = for | | | | | | | |
| 16 | 95737_at | Cluster Incl AW120814: UI-M-BH2.3-aoa-b-07-0-UI s1 Mus musculus cDNA, 3 end/clone = UI-M-BH2 3-aoa-b-07-0-UI/ | 547.5 | P | 863.2 | P | 878 | P | 773.4 |
| | | clone_end = 3/gb = AW120814/gi = 6096147/ug = Mm 29750/len = 314/STRA = for | | | | | | | |
| 16 | 96338_at | Cluster Incl AW125059: UI-M-BH2.1-apx-c-07-0-UI s1 Mus musculus cDNA, 3 end/clone = UI-M-BH2 1-apx-c-07-0-UI/ | 989.6 | P | 1034.7 | P | 998.9 | P | 1015 |
| | | clone_end = 3/gb = AW125059/gi = 6100589/ug = Mm 29978/len = 448/STRA = for | | | | | | | |
| 16 | 96845_at | Cluster Incl AI849577 UI-M-AH1-agr-d-03-0-UI.s1 Mus musculus cDNA, 3 end/clone = UI-M-AH1-agr-d-03-0-UI/ | 259.1 | P | 213.1 | P | 213.3 | P | 185.8 |
| | | clone_end = 3/gb = AI849577/gi = 5493483/ug = Mm 30023/len = 242/STRA = for | | | | | | | |
| 16 | 96894_at | Cluster Incl AW122685 UI-M-BH2.2-aot-a-06-0-UI s1 Mus musculus cDNA, 3 end/clone = UI-M-BH2 2-aot-a-06-0-UI/ | 453.9 | P | 478.5 | P | 472.3 | P | 373.9 |
| | | clone_end = 3/gb = AW122685/gi = 6098215/ug = Mm 30103/len = 445/STRA = for | | | | | | | |
| 16 | 96934_at | Cluster Incl AW049074 UI-M-BH1-amt-d-01-0-UI s1 Mus musculus cDNA, 3 end/clone = UI-M-BH1-aml-d-01-0-UI/ | 1049.4 | P | 1083.9 | P | 981.5 | P | 984.1 |
| | | clone_end = 3/gb = AW049074/gi = 5909603/ug = Mm 30179/len = 285/STRA = for | | | | | | | |
| 16 | 97463_g_at | Cluster Incl AI847584 UI-M-AP1-agg-a-06-0-UI s1 Mus musculus cDNA, 3 end/clone = UI-M-AP1-agg-a-06-0-UI/ | 2305.4 | P | 2451.2 | P | 2821.4 | P | 2284.7 |
| | | clone_end = 3/gb = AI847584/gi = 5491490/ug = Mm 3035/len = 340/STRA = for | | | | | | | |
| 16 | 98047_at | Cluster Incl AI851583 UI-M-BH0-aki-g-07-0-UI s1 Mus musculus cDNA, 3 end/clone = UI-M-BH0-aki-g-07-0-UI/ | 346.6 | P | 458.7 | P | 376.8 | P | 419.7 |
| | | clone_end = 3/gb = AI851583/gi = 5495489/ug = Mm.34261/len = 460/STRA = for | | | | | | | |
| 16 | 98070_at | Cluster Incl AI844794 UI-M-AH1-agx-a-02-0-UI s1 Mus musculus cDNA, 3 end/clone = UI-M-AH1-agx-a-02-0-UI/ | 832.2 | P | 914.9 | P | 1018.1 | P | 816.6 |
| | | clone_end = 3/gb = AI844794/gi = 5488700/ug = Mm 34456/len = 394/STRA = for | | | | | | | |
| 16 | 98522_at | Cluster Incl AI839158 UI-M-AO0-ach-f-03-0-UI s1 Mus musculus cDNA, 3 end/clone = UI-M-AO0-ach-f-03-0-UI/ | 1130.3 | P | 1568.6 | P | 1227.7 | P | 1326.9 |
| | | clone_end = 3/gb = AI839158/gi = 5473371/ug = Mm 35812/len = 419/STRA = for | | | | | | | |
| 16 | 98528_at | Cluster Incl AI854901 UI-M-BH0-akb-h-06-0-UI s1 Mus musculus cDNA, 3 end/clone = UI-M-BH0-akb-h-06-0-UI/ | 1186.6 | P | 1411.9 | P | 1251.1 | P | 1231 |
| | | clone_end = 3/gb = AI854901/gi = 5498807/ug = Mm 35837/len = 398/STRA = for | | | | | | | |
| 16 | 98625_s_at | Cluster Incl M84147 Mouse alcohol dehydrogenase-B2 (Adh-2) mRNA, putative cds/cds = (39,1163)/gb = M84147/ | 824.2 | P | 1020.1 | P | 961.7 | P | 740.1 |
| | | gi = 191721/ug = Mm.3874/len = 1360/STRA = for | | | | | | | |
| 16 | 98633_at | Cluster Incl AI854863 UI-M-BH0-akb-d-12-0-UI s1 Mus musculus cDNA, 3 end/clone = UI-M-BH0-akb-d-12-0-UI/ | 602.1 | P | 895.5 | P | 1112.3 | P | 595.2 |
| | | clone_end = 3/gb = AI854863/gi = 5498769/ug = Mm 38801/len = 419/STRA = for | | | | | | | |
| 16 | 98635_at | Cluster Incl AI854629 UI-M-BH0-ake-e-11-0-UI s1 Mus musculus cDNA, 3 end/clone = UI-M-BH0-ake-e-11-0-UI/ | 469.2 | P | 592.2 | P | 534 | P | 412.2 |
| | | clone_end = 3/gb = AI854629/gi = 5498535/ug = Mm 38802/len = 492/STRA = for | | | | | | | |
| 16 | 99176_at | Cluster Incl AI843393: UI-M-AQ1-adz-g-04-0-UI s1 Mus musculus cDNA, 3 end/clone = UI-M-AQ1-adz-g-04-0-UI/ | 319.9 | P | 590.3 | P | 433.6 | P | 411.3 |
| | | clone_end = 3/gb = AI843393/gi = 5477606/ug = Mm 41785/len = 394/STRA = for | | | | | | | |
| 16 | 99191_at | Cluster Incl AI844939 UI-M-BG0-ahv-g-09-0-UI s1 Mus musculus cDNA, 3 end/clone = UI-M-BG0-ahv-g-09-0-UI/ | 1030.4 | P | 1572.9 | P | 1746.9 | P | 964.2 |
| | | clone_end = 3/gb = AI844939/gi = 5488845/ug = Mm 41906/len = 413/STRA = for | | | | | | | |
| 16 | 99617_at | Cluster Incl AI846299: UI-M-AK1-aez-f-12-0-UI s1 Mus musculus cDNA, 3 end/clone = UI-M-AK1-aez-f-12-0-UI/ | 1056.4 | P | 1172.1 | P | 966.8 | P | 1006.6 |
| | | clone_end = 3/gb = AI846299/gi = 5490205/ug = Mm 43040/len = 335/STRA = for | | | | | | | |
| 16 | 100125_at | Cluster Incl U43918 Mus musculus proliferation-associated protein 1 mRNA, complete cds/cds = (266,1450)/gb = U43918/ | 389.7 | P | 721.4 | P | 537.3 | P | 488.9 |
| | | gi = 1167966/ug = Mm 4742/len = 1714/STRA = for | | | | | | | |
| 16 | 100594_at | Cluster Incl AB008895: Mus musculus mRNA for mGpI1p, complete cds/cds = (140,1885)/gb = AB008895/gi = 2641975/ | 209.6 | P | 246.1 | P | 213.8 | P | 222.8 |
| | | ug = Mm. 6354/len = 3062/STRA = for | | | | | | | |
| 16 | 100633_at | Cluster Incl AW124268: UI-M-BH2 1-aph-h-08-0-UI s1 Mus musculus cDNA, 3 end/clone = UI-M-BH2 1-aph-h-08-0-UI/ | 708.6 | P | 1255 | P | 1015.9 | P | 928.3 |
| | | clone_end = 3/gb = AW124268/gi = 6099798/ug = Mm 6696/len = 385/STRA = for | | | | | | | |
| 16 | 101090_at | Cluster Incl L29454: Fibnllin 1/cds = (2379,10994)/gb = L29454/gi = 575509/ug = Mm.735/len = 11953/STRA = for | 977.9 | P | 1777.7 | P | 1447.5 | P | 963.3 |
| 16 | 101585_at | Cluster Incl AF042491 Mus musculus putative membrane associated progesterone receptor component mRNA, complete | 696.7 | P | 866.9 | P | 762.4 | P | 561.6 |
| | | cds/cds = (13,600)/gb = AF042491/gi = 2801792/ug = Mm 9052/len = 1786/STRA = for | | | | | | | |
| 16 | 101989_at | Cluster Incl AW125380 UI-M-BH2 3-aqh-b-06-0-UI.s1 Mus musculus cDNA, 3 end/clone = UI-M-BH2 3-aqh-b-06-0-UI/ | 2097.5 | P | 2963.2 | P | 2771.1 | P | 2139.7 |
| | | clone_end = 3/gb = AW125380/gi = 6100910/ug = Mm 972/len = 269/STRA = for | | | | | | | |
| 16 | 102002_at | Cluster Incl AI836406 UI-M-AP0-abi-g-05-0-UI s1 Mus musculus cDNA, 3 end/clone = UI-M-AP0-abi-g-05-0-UI/ | 461 | P | 523.3 | P | 511.5 | P | 374 |
| | | clone_end = 3/gb = AI836406/gi = 5470619/ug = Mm 99026/len = 435/STRA = for | | | | | | | |
| 16 | 102061_at | Cluster Incl AF100171: Mus musculus myelodysplasia/myeloid leukemia factor 1 (Mlf1) mRNA, complete cds/cds = (60,863)/ | 159.6 | P | 222.1 | P | 183 | P | 149.1 |
| | | gb = AF100171/gi = 4106525/ug = Mm.10414/len = 879/STRA = for | | | | | | | |
| 16 | 102384_at | Cluster Incl AI842968 UI-M-AO1-aem-g-04-0-UI.s1 Mus musculus cDNA, 3 end/clone = UI-M-AO1-aem-g-04-0-UI/ | 146.1 | P | 192.6 | P | 142.9 | P | 101.9 |
| | | clone_end = 3/gb = AI842968/gi = 5477181/ug = Mm.12184/len = 467/STRA = for | | | | | | | |
| 16 | 102794_at | Cluster Incl Z80112 Mus musculus lcr-1 gene/cds = (0,1073)/gb = Z80112/gi = 2632100/ug = Mm 1401/len = 1180/STRA = for | 77.6 | P | 141.4 | P | 115.7 | P | 102.5 |
| 16 | 102859_at | Cluster Incl AW121304: UI-M-BH2.3-aog-g-02-0-UI s1 Mus musculus cDNA, 3 end/clone = UI-M-BH2 3-aog-g-02-0-UI/ | 290.3 | P | 336.7 | P | 405.4 | P | 306.8 |
| | | clone_end = 3/gb = AW121304/gi = 6096637/ug = Mm 14992/len = 261/STRA = for | | | | | | | |
| 16 | 103556_at | Cluster Incl AI840158 UI-M-AO0-acc-d-08-0-Ul.s1 Mus musculus cDNA, 3 end/clone = UI-M-AO0-aoc-d-08-0-UI/ | 920.4 | P | 1494.9 | P | 1434.4 | P | 843.8 |
| | | clone_end = 3/gb = AI840158/gi = 5474371/ug = Mm 19081/len = 406/STRA = for | | | | | | | |
| 16 | 103570_at | Cluster Incl AI315647 uj31c11 x1 Mus musculus cDNA, 3 end/clone = IMAGE-1921556/clone_end = 3/gb = AI315647/ | 317.6 | P | 564.2 | P | 398 | P | 161.9 |
| | | gi = 4030914/ug = Mm 19310/len = 467/STRA = for | | | | | | | |
| 16 | 103592_at | Cluster Incl AB019374 Mus musculus mRNA for MEK5, complete cds/cds = (596,1942)/gb = AB019374/gi = 5360522/ | 246.1 | P | 309.7 | P | 274.2 | P | 208.5 |
| | | ug = Mm 19947/len = 2259/STRA = for | | | | | | | |
| 16 | 103650_at | Cluster Incl AI846887 UI-M-AK1-aey-a-01-0-UI s1 Mus musculus cDNA, 3 end/clone = UI-M-AK1-aey-a-01-0-UI/ | 276 | P | 382.3 | P | 380.7 | P | 323.3 |
| | | clone_end = 3/gb = AI846887/gi = 5490793/ug = Mm 20414/len = 320/STRA = for | | | | | | | |
| 16 | 103812_at | Cluster Incl AF047838: Mus musculus calcium-sensitive chlonde conductance protein-1 (mCLCA1) mRNA, complete cds/ | 183.6 | P | 204.9 | P | 286.3 | P | 139.8 |
| | | cds = (17,2725)/gb = AF047838/gi = 3925280/ug = Mm 20897/len = 3022/STRA = rev | | | | | | | |
| 16 | 103829_at | Cluster Incl AF061529 Mus musculus rjs (rjs) mRNA, complete cds/cds = (119,14629)/gb = AF061529/gi = 3414808/ | 20.7 | P | 52.7 | P | 86 | P | 59.1 |
| | | ug = Mm 20929/len = 15264/STRA = for | | | | | | | |
| 16 | 104345_at | Cluster Incl AF014461 Mus musculus EXO70 protein (Exo70) mRNA, complete cds/cds = (0,2093)/gb = AF014461/ | 177.9 | P | 186.4 | P | 329.1 | P | 187.2 |
| | | gi = 2352997/ug = Mm 22530/len = 2094/STRA = for | | | | | | | |
| 16 | 104365_at | Cluster Incl AW046162 UI-M-BH1-akw-b-04-0-UI s1 Mus musculus cDNA, 3 end/clone = UI-M-BH1-akw-b-04-0-UI/ | 376.2 | P | 505.4 | P | 465.7 | P | 479.7 |
| | | clone_end = 3/gb = AW046162/gi = 5906691/ug = Mm 22615/len = 456/STRA = for | | | | | | | |
| 16 | 104572_at | Cluster Incl AI852047: UI-M-BH0-aja-b-01-0-UI s1 Mus musculus cDNA, 3 end/clone = UI-M-BH0-aja-b-01-0-UI/ | 418.1 | P | 455.7 | P | 437.6 | P | 402.5 |
| | | clone_end = 3/gb = AI852047/gi = 5495953/ug = Mm 23910/len = 523/STRA = for | | | | | | | |
| 16 | 104725_at | Cluster Incl AW060401 UI-M-BH1-anj-a-06-0-UI s1 Mus musculus cDNA, 3 end/clone = UI-M-BH1-anj-a-06-0-UI/ | 417.8 | P | 520.4 | P | 433.8 | P | 395.3 |
| | | clone_end = 3/gb = AW060401/gi = 6008152/ug = Mm 24574/len = 467/STRA = for | | | | | | | |
| 16 | 104746_at | Cluster IncI AF040252 Mus musculus FK506-binding protein (FKBP23) mRNA, complete cds/cds = (56,712)/gb = AF040252/ | 338.1 | P | 306.2 | P | 343.1 | P | 239.9 |
| | | gi = 2827254/ug = Mm 24720/len = 866/STRA = for | | | | | | | |
| 16 | 94331_at | Cluster Incl L47650 Signal transducer and activator of transcription 6/cds = (279,2792)/gb = L47650/gi = 1008876/ | 276.7 | P | 410.6 | P | 386.6 | P | 330.7 |
| | | ug = Mm 2489/len = 3213/STRA = for | | | | | | | |
| 16 | 94348_f_at | Cluster Incl AW124044 UI-M-BH2.1-apn-d-10-0-UI s1 Mus musculus cDNA, 3 end/clone = UI-M-BH2 1-apn-d-10-0-UI/ | 401.6 | P | 612.5 | P | 376.3 | P | 464.6 |
| | | clone_end = 3/gb = AW124044/gi = 6099574/ug = Mm.25169/len = 408/STRA = for | | | | | | | |
| 16 | 94392_f_at | Cluster Incl U22516: Angiogenin/cds = (0,437)/gb = U22516/gi = 726325/ug = Mm 2566/len = 438/STRA = for | 133.8 | P | 166.1 | P | 239.8 | P | 130.8 |
| 16 | 94422_at | Cluster Incl AI842938 UI-M-AO1-aem-d-04-0-UI.s1 Mus musculus cDNA, 3 end/clone = UI-M-AO1-aem-d-04-0-UI/ | 99.3 | P | 218 | P | 163.2 | P | 167.4 |
| | | clone_end = 3/gb = AI842938/gi = 5477151/ug = Mm.26242/len = 303/STRA = for | | | | | | | |
| 16 | 94930_at | Cluster Incl L07803: Thrombospondin 2/cds = (218,3736)/gb = L07803/gi = 340421/ug = Mm 26688/len = 4108/STRA = for | 504.7 | P | 649.1 | P | 520.5 | P | 489.6 |
| 16 | 94976_at | Cluster Incl AI845877 Ul-M-AO1-aeg-g-12-0-UI s1 Mus musculus cDNA, 3 end/clone = UI-M-AO1-aog-g-12-0-UI/ | 586 | P | 578.1 | P | 402.2 | P | 344.3 |
| | | clone_end = 3/gb = AI845877/gi = 5489783/ug = Mm.27259/len = 409/STRA = rev | | | | | | | |
| 16 | 94995_at | Cluster Incl AI854331 UI-M-BG1-aic-b-07-0-UI s1 Mus musculus cDNA, 3 end/clone = UI-M-BG1-aic-b-07-0-UI/ | 852.7 | P | 1141.7 | P | 1009.3 | P | 526.2 |
| | | clone_end = 3/gb = AI854331/gi = 5498237/ug = Mm.27337/len = 404/STRA = rev | | | | | | | |
| 16 | 95014_at | Cluster Incl AA794247: vu65b08 r1 Mus musculus cDNA, 5 end/clone = IMAGE-1196247/clone_end = 5/gb = AA794247/ | 648.9 | P | 730. 7 | P | 710.5 | P | 643.3 |
| | | gi = 2857202/ug = Mm.27445/len = 496/STRA = for | | | | | | | |
| 16 | 95529_at | Cluster Incl U58884 Mus musculus SH3-containing protein SH3P7 mRNA, complete cds similar to Human Drebrin/ | 735.8 | P | 658.5 | P | 816.2 | P | 632.5 |
| | | cds = (30,1331)/gb = U58884/gi = 1407654/ug = Mm 27622/len = 2291/STRA = rev | | | | | | | |
| 16 | 95565_at | Cluster Incl AI852873 UI-M-BH0-ajk-f-03-0-Ul.s1 Mus musculus cDNA, 3 end/clone = UI-M-BH0-ajk-f-03-0-UI/clone_end = 3/ | 508.5 | P | 610.3 | P | 488.1 | P | 512.4 |
| | | gb = AI852873/gi = 5496779/ug = Mm 27787/len = 327/STRA = for | | | | | | | |
| 16 | 95590_at | Cluster Incl AA615951: vo95c11.r1 Mus musculus cDNA, 5 end/clone = IMAGE-1066868/clone_end = 5/gb = AA615951/ | 203.5 | P | 257. 3 | P | 192.9 | P | 198.1 |
| | | gi = 2503187/ug = Mm 27890/len = 621/STRA = for | | | | | | | |
| 16 | 95597_at | Cluster Incl M34141 Prostaglandin-endoperoxide synthase 1/cds = (36,1844)/gb = M34141/gi = 200302/ug = Mm.2792/ | 193.6 | P | 217.2 | P | 179.6 | P | 161.5 |
| | | len = 2757/STRA = for | | | | | | | |
| 16 | 95617_at | Cluster Incl U36799 Retinoblastoma-like 2/cds = (19,3396)/gb = U36799/gi = 1045519/ug = Mm 28027/len = 4806/STRA = for | 90.7 | P | 106.2 | P | 111.7 | P | 82.1 |
| 16 | 95622_at | Cluster Incl AW123907 UI-M-BH2 1-app-h-01-0-UI.s1 Mus musculus cDNA, 3 end/clone = UI-M-BH2.1-app-h-01-0-UI/ | 353.4 | P | 419.6 | P | 474 | P | 328.2 |
| | | clone_end = 3/gb = AW123907/gi = 6099437/ug = Mm 28047/len = 326/STRA = for | | | | | | | |
| 16 | 95630_at | Cluster Incl AW122852 UI-M-BH2 1-aoz-g-07-0-UI s1 Mus musculus cDNA, 3 end/clone = UI-M-BH2 1-aoz-g-07-0-UI/ | 395.9 | P | 596.5 | P | 656 | P | 391.4 |
| | | clone_end = 3/gb = AW122852/gi = 6098382/ug = Mm 28080/len = 351/STRA = rev | | | | | | | |
| 16 | 96127_at | Cluster Incl AW048730: UI-M-BH1-amy-e-02-0-UI s1 Mus musculus cDNA, 3 end/clone = UI-M-BH1-amy-e-02-0-UI/ | 753.3 | P | 952.7 | P | 1026.9 | P | 666.3 |
| | | clone_end = 3/gb = AW048730/gi = 5909259/ug = Mm 28119/len = 536/STRA = for | | | | | | | |
| 16 | 96765_at | Cluster Incl AW120874: UI-M-BH2.3-aoa-h-02-0-UI.s1 Mus musculus cDNA, 3 end/clone = UI-M-BH2.3-aoa-h-02-0-UI/ | 1996.9 | P | 2062.8 | P | 1492.1 | P | 1027.6 |
| | | clone_end = 3/gb = AW120874/gi = 6096207/ug = Mm.29009/len = 298/STRA = for | | | | | | | |
| 16 | 96802_at | Cluster Incl AW124656: UI-M-BH2 1-apw-e-04-0-U1.s1 Mus musculus cDNA, 3 end/clone = UI-M-BH2.1-apw-e-04-0-UI/ | 134.5 | P | 129.6 | P | 154.2 | P | 124.5 |
| | | clone_end = 3/gb = AW124656/gi = 6100186/ug = Mm.29193/len = 368/STRA = for | | | | | | | |
| 16 | 97977_at | Cluster Incl AA645293 vs81a07.r1 Mus musculus cDNA, 5 end/clone = IMAGE-1152660/clone_end = 5/gb = AA645293/ | 453.1 | P | 562.6 | P | 514.7 | P | 366.4 |
| | | gi = 2571722/ug = Mm 3116/len = 276/STRA = for | | | | | | | |
| 16 | 98468_r_at | Cluster Incl AI316859: ul35b03 y1 Mus musculus cDNA, 5 end/clone = IMAGE-1853261/clone_end = 5/gb = AI316659/ | 240.4 | P | 293.1 | P | 291.8 | P | 202 |
| | | gi = 4032126/ug = Mm 34879/len = 500/STRA = for | | | | | | | |
| 16 | 98511_at | Cluster Incl L17076 HnRNP-associated with lethal yellow/cds = UNKNOWN/gb = L17076/gi = 348995/ug = Mm 3591/ | 505.7 | P | 657.3 | P | 551.5 | P | 351.4 |
| | | len = 1517/STRA = for | | | | | | | |
| 16 | 99010_at | Cluster Incl AB024538 Mus musculus Islr(immunoglobulin superfamily containing leucine-nch repeat) mRNA, complete cds/ | 498.1 | P | 621.3 | P | 834.4 | P | 230.9 |
| | | cds = (89,1375)/gb = AB024538/gi = 6172374/ug = Mm 38426/len = 2038/STRA = for | | | | | | | |
| 16 | 99029_at | Cluster Incl AF077950: Mus musculus protein inhibitor of activated STAT protein PIAS1 mRNA, complete cds/cds = (0,1955)/ | 273.4 | P | 391.3 | P | 399.4 | P | 227.9 |
| | | gb = AF077950/gi = 3643104/ug = Mm 38869/len = 1956/STRA = for | | | | | | | |
| 16 | 99067_at | Cluster Incl X59846 M. musculus GAS 6 mRNA associated with growth-arrest/cds = (167,2188)/gb = X59846/gi = 407060/ | 308.9 | P | 388 | P | 430.2 | P | 222.8 |
| | | ug = Mm 3982/len = 2573/STRA = rev | | | | | | | |
| 16 | 99476_at | Cluster Incl AJ131395: Mus musculus mRNA for collagen type XIV, partial/cds = (0,380)/gb = AJ131395/gi = 5042293/ | 561.1 | P | 782.5 | P | 783.3 | P | 499.2 |
| | | ug = Mm.41332/len = 1171/STRA = for | | | | | | | |
| 16 | 99518_at | Cluster Incl AW121179 UI-M-BH2 3-aok-a-06-0-UI s1 Mus musculus cDNA, 3 end/clone = UI-M-BH2 3-aok-a-06-0-UI/ | 652.3 | P | 1316.8 | P | 796.7 | P | 817.8 |
| | | clone_end = 3/gb = AW121179/gi = 6096512/ug = Mm 41931/len = 479/STRA = for | | | | | | | |
| 16 | 99940_at | Cluster Incl AW212667 uo10c01 x1 Mus musculus cDNA, 3 end/clone = IMAGE-2599104/clone_end = 3/gb = AW212667/ | 206.3 | P | 202.7 | P | 270.7 | P | 257.4 |
| | | gi = 6518780/ug = Mm 435/len = 464/STRA = for | | | | | | | |
| 16 | 99947_at | Cluster Incl U55861 Mus musculus RNA binding protein TIAR mRNA, complete cds/cds = (133,1311)/gb = U55861/ | 384.3 | P | 500.8 | P | 334 | P | 341.9 |
| | | gi = 1592562/ug = Mm 4363/len = 1543/STRA = for | | | | | | | |
| 16 | 100443_at | Cluster Incl AF031467 Branched chain aminotransferase 2, mitochondrial/cds = (135,1196)/gb = AF031467/gi = 3298578/ | 159.7 | P | 368.7 | P | 170.2 | P | 249.9 |
| | | ug = Mm 4811/len = 1200/STRA = for | | | | | | | |
| 16 | 100477_at | Cluster Incl M32486 Mouse 19.5 mRNA, complete cds/cds = (54,875)/gb = M32486/gi = 191495/ug = Mm 4992/len = 1183/ | 1572.6 | P | 1831.6 | P | 1824.5 | P | 1031.3 |
| | | STRA = for | | | | | | | |
| 16 | 101416_f_at | Cluster Incl L76155 Mus musculus Bat-4 gene, complete cds/cds = (40,1158)/gb = L76155/gi = 1196643/ug = Mm 7405/ | 607.3 | P | 918.7 | P | 650.9 | P | 612.8 |
| | | len = 1400/STRA = for | | | | | | | |
| 16 | 104408_s_at | Cluster Incl L35032 SRY-box containing gene 18/cds = (88,1221)/gb = L35032/gi = 1663531/ug = Mm 2878/len = 1585/ | 352.6 | P | 534.7 | P | 365.8 | P | 321.3 |
| | | STRA = for | | | | | | | |
| 16 | 104445_at | Cluster Incl AW046694 UI-M-BH1-alm-d-07-0-UI s1 Mus musculus cDNA, 3 end/clone = UI-M-BH1-alm-d-07-0-UI/ | 1172.8 | P | 1435.8 | P | 894.5 | P | 865.4 |
| | | clone_end = 3/gb = AW046694/gi = 5907223/ug = Mm 2935/len = 253/STRA = for | | | | | | | |
| 16 | 92207_at | Cluster Incl U08210 Elastin/cds = (0,2582)/gb = U08210/gi = 473273/ug = Mm 34170/len = 2651/STRA = for | 660.2 | P | 871.1 | P | 697.8 | P | 624.3 |
| 16 | 92224_at | Cluster Incl X79199 Tetranectin (plasminogen-binding protein)/cds = (73,681)/gb = X79199/gi = 671501/ug = Mm 34588/ | 409.5 | P | 859.5 | P | 898.9 | P | 535 |
| | | len = 988/STRA = rev | | | | | | | |
| 16 | 92375_at | Cluster Incl AW211207 ut29a03 y1 Mus musculus cDNA, 5 end/clone = IMAGE-2099692/clone_end = 5/gb = AW211207/ | 219 | P | 320.5 | P | 334.5 | P | 158.8 |
| | | gi = 6517147/ug = Mm 4002/len = 531/STRA = for | | | | | | | |
| 16 | 93443_at | Cluster Incl AW212271 uo84g08 x1 Mus musculus cDNA, 3 end/clone = IMAGE-2649278/clone_end = 3/gb = AW212271/ | 417.8 | P | 494.7 | P | 414.5 | P | 336.6 |
| | | gi = 6515306/ug = Mm 6671/len = 420/STRA = for | | | | | | | |
| 16 | 93472_at | Cluster Incl AA796989: vt01a04 r1 Mus musculus cDNA, 5 end/clone = IMAGE-1154478/clone_end = 5/gb = AA796989/ | 1498.2 | P | 1861.8 | P | 1991.7 | P | 1369.2 |
| | | gi = 2859944/ug = Mm.7049/len = 428/STRA = for | | | | | | | |
| 16 | 93485_at | Cluster Incl AI844911 UI-M-BG0-ahv-d-10-0-UI s1 Mus musculus cDNA, 3 end/clone = UI-M-BG0-ahv-d-10-0-UI/ | 130.2 | P | 215.9 | P | 154.1 | P | 77.2 |
| | | clone_end = 3/gb = AI844911/gi = 5488617/ug = Mm 7172/len = 399/STRA = for | | | | | | | |
| 16 | 93615_at | Cluster Incl AF020200 Mus musculus transcription factor PBX3b (PBX3b) mRNA, complete cds/cds = (118,1173)/ | 106.1 | P | 197.6 | P | 130.7 | P | 128.2 |
| | | gb = AF020200/gi = 2432016/ug = Mm.7331/len = 2467/STRA = for | | | | | | | |
| 16 | 93874_s_at | Cluster Incl U69491: lnterleukin 11 receptor, alpha chain 2/cds = (301,1599)/gb = U69491/gi = 1916003/ug = Mm 88405/ | 665.7 | P | 883.2 | P | 624.1 | P | 556.5 |
| | | len = 1975/STRA = for | | | | | | | |
| 16 | 99812_at | Cluster Incl X92523 Calpain 3/cds = (0,2465)/gb = X92523/gi = 1246778/ug = Mm 57235/len = 2466/STRA = for | 223.2 | P | 264.8 | P | 263.3 | P | 227.8 |
| 16 | 94642_at | Cluster Incl U07951 Mus musculus BALB/c GDP-dissociation inhibitor (GDI-2) mRNA, complete cds/cds = (289,1626)/ | 225.4 | P | 213 | P | 196.9 | P | 164.5 |
| | | gb = U07951/gi = 1486479/ug = Mm 8070/len = 1676/STRA = for | | | | | | | |
| 16 | 93835_at | Cluster Incl AI843222: UI-M-AK1-aet-g-05-0-UI s1 Mus musculus cDNA, 3 end/clone = UI-M-AK1-ael-g-05-0-UI/ | 213.1 | P | 308.8 | P | 238.4 | P | 90.5 |
| | | clone_end = 3/gb = AI843222/gi = 5477435/ug = Mm.21580/len = 373/STRA = for | | | | | | | |
| 16 | 94079_at | Cluster Incl X61452 M musculus mRNA for H5 clone/cds = (35,1471)/gb = X61452/gi = 51202/ug = Mm 2214/len = 1649/ | 128.9 | P | 242.6 | P | 173.6 | P | 73.2 |
| | | STRA = for | | | | | | | |
| 16 | 94275_at | Cluster Incl U34691 Uroporphynnogen decarboxylase/cds = (281,1384)/gb = U34691/gi = 1498642/ug = Mm 22494/len = 1454/ | 187 | P | 244.1 | P | 240 | P | 194.4 |
| | | STRA = for | | | | | | | |
| 16 | 94304_at | Cluster Incl X13460: Annexin VI, p68/cds = (33,2054)/gb = X13460/gi = 53580/ug = Mm 22619/len = 2402/STRA = for | 741.3 | P | 1115.6 | P | 914.6 | P | 772.8 |
| 16 | 97835_at | Cluster Incl AI842828 UI-M-AP1-agi-h-10-0-UI s1 Mus musculus cDNA, 3 end/clone = UI-M-Ap1-agi-h-10-0-UI/ | 413 | P | 663.5 | P | 811.3 | P | 698.6 |
| | | clone_end = 3/gb = AI842828/gi = 5477041/ug = Mm28231/len = 175/STRA = for | | | | | | | |
| 16 | 98923_at | Cluster Incl AI852608 UI-M-BH0-alu-d-07-0-UI s1 Mus musculus cDNA, 3 end/clone = UI-M-BH0-alu-d-07-0-UI/ | 89.5 | P | 155.9 | P | 110.9 | P | 124.2 |
| | | clone_end = 3/gb = AI852608/gi = 5496514/ug = Mm 28630/len = 273/STRA = for | | | | | | | |
| 16 | 94459_at | Cluster Incl AA681764 v161b04 r1 Mus musculus cDNA, 5 end/clone = IMAGE-1167535/clone_end = 5/gb = AA681764/ | 117.2 | P | 141.3 | P | 92.4 | P | 160.6 |
| | | gi = 2663904/ug = Mm.28818/len = 441/STRA = for | | | | | | | |
| 16 | 96296_at | Cluster Incl AI843685 UI-M-AK1-aer-c-07-0-UI s1 Mus musculus cDNA, 3 end/clone = UI-M-AK1-aer-c-07-0-UI/ | 431.1 | P | 431.1 | P | 399.9 | P | 424.9 |
| | | clone_end = 3/gb = AI843685/gi = 5477898/ug = Mm.29904/len = 305/STRA = for | | | | | | | |
| 16 | 99630_at | Cluster Incl AW060257 UI-M-BH1-amv-f-08-0-UI.s1 Mus musculus cDNA, 3 end/clone = UI-M-BH1-amv-f-08-0-UI/ | 203.9 | P | 294.7 | P | 331.7 | P | 199.9 |
| | | clone_end = 3/gb = AW060257/gi = 6008048/ug = Mm.43397/1en = 434/STRA = for | | | | | | | |
| 16 | 101036_at | Cluster Incl AW047047 UI-M-BH1-alr-c-02-0-UI s1 Mus musculus cDNA, 3 end/clone = UI-M-BH1-alr-c-02-0-UI/ | 584.1 | P | 482.1 | P | 596.5 | P | 575 |
| | | clone_end = 3/gb = AW047047/gi = 5907576/ug = Mm.6932/len = 334/STRA = for | | | | | | | |
| 16 | 103030_at | Cluster Incl L31397: Dynamin/cds = (73,2658)/gb = L31397/gi = 487856/ug = Mm 16080/len = 3924/STRA = rev | 188.9 | P | 320 | P | 365.2 | P | 136.4 |
| 16 | 104610_at | Cluster Incl AW050015: UI-M-BH1-amm-a-01-0-UI.s1 Mus musculus cDNA, 3 end/clone = UI-M-BH1-amm-a-01-0-UI/ | 136.3 | P | 188.5 | P | 167.3 | P | 130.3 |
| | | clone_end = 3/gb = AW050015/gi = 5910544/ug = Mm.2415/len = 503/STRA = rev | | | | | | | |
| 16 | 104616_g_at | Cluster Incl M96265: Mus musculus galactose-1-phosphate uridyl transferase (GALT) mRNA, complete cds/cds = (131,1312)/ | 79 | P | 182.5 | P | 95.2 | P | 57.3 |
| | | gb = M96265/gi = 193421/ug = Mm 2420/len = 1373/STRA = for | | | | | | | |
| 16 | 94428_at | Cluster Incl AI842668 UI-M-AO1-aeo-a-05-0-UI s1 Mus musculus cDNA, 3 end/clone = UI-M-AO1-aeo-a-05-0-UI/ | 168.4 | P | 275.5 | P | 250.8 | P | 169.7 |
| | | clone_end = 3/gb = AI842668/gi = 5476881/ug = Mm 2644/len = 378/STRA = for | | | | | | | |
| 16 | 95537_at | Cluster Incl AB019577 Mus musculus mRNA for UNC-51-like kinase (ULK) 2, complete cds/cds = (41,3154)/gb = AB019577/ | 145.2 | P | 116.1 | P | 130.5 | P | 76.2 |
| | | gi = 4760560/ug = Mm 27664/len = 5275/STRA = for | | | | | | | |
| 16 | 96728_at | Cluster Incl AI844151 UI-M-AL1-ahj-f-07-0-UI.s1 Mus musculus cDNA, 3 end/clone = UI-M-AL1-ahj-f-07-0-UI/clone_end = 3/ | 110.2 | A | 248.8 | P | 356.9 | P | 101.1 |
| | | gb = AI844151/gi = 5488057/ug = Mm 28850/len = 317/STRA = rev | | | | | | | |
| 16 | 99929_at | Cluster Incl U72141: Exostoses (multiple) 2/cds = (191,2347)/gb = U72141/gi = 1619953/ug = Mm 4338/len = 2864/STRA = for | 410.1 | P | 457.5 | P | 442.6 | P | 339.7 |
| 16 | 99992_at | Cluster Incl AI286698 ub84b09 r1 Mus musculus cDNA, 5 end/clone = IMAGE-1395161/clone_end = 5/gb = AI286698/ | 283.9 | P | 291.7 | P | 310.8 | P | 204.3 |
| | | gi = 3926451/ug = Mm.4481/len = 200/STRA = for | | | | | | | |
| 16 | 100468_g_at | Cluster Incl X57667 M musculus LYL gene (clone L6)/cds = (465,1301)/gb = X57687/gi = 52965/ug = Mm 4925/len = 1686/ | 147.5 | P | 158.2 | P | 128.7 | P | 132.6 |
| | | STRA = for | | | | | | | |
| 16 | 101446_at | Cluster Incl AF004428, Turnor protein D52-like 1/cds = (191,805)/gb = AF004428/gi = 2895080/ug = Mm.7821/len = 1267/ | 128.5 | P | 198.9 | P | 217.3 | P | 103.4 |
| | | STRA = for | | | | | | | |
| 16 | 102720_at | Cluster Incl X71426 Endothelial-specific receptor tyrosine kinase/cds = (86 3454)/gb = X71426/gi = 296612/ug = Mm 14313/ | 109.1 | P | 175.3 | P | 140.5 | P | 117 |
| | | len = 3490/STRA = for | | | | | | | |
| 16 | 103536_at | Cluster Incl AB017270 Mus musculus TMEFF2 mRNA, complete cds/cds = (388,1512)/gb = AB017270/gi = 6518464/ | 94.2 | A | 149.5 | P | 144.9 | P | −2.7 |
| | | ug = Mm 23304/len = 1740/STRA = for | | | | | | | |
| 16 | 103922_f_at | Cluster Incl AI839690 UI-M-AN0-acp-e-04-0-UI s1 Mus musculus cDNA, 3 end/clone = UI-M-AN0-acp-e-04-0-UI/ | 145.1 | P | 190.8 | P | 170.8 | P | 106.4 |
| | | clone_end = 3/gb = AI839690/gi = 5473903/ug = Mm 25034/len = 250/STRA = rev | | | | | | | |
| 16 | 104680_at | Cluster Incl AJ250489 Mus musculus mRNA for receptor activity modifying protein 1 (Ramp1 gene)/cds = (59,505)/ | 439.6 | P | 664.3 | P | 529.8 | P | 318.5 |
| | | gb = AJ250489/gi = 6119624/ug = Mm 3272/len = 844/STRA = for | | | | | | | |
| 16 | 92366_at | Cluster Incl U12147.Laminin, alpha 2/cds = (54,9374)/gb = U12147/gi = 699109/ug = Mm 3987/len = 9511/STRA = for | 333.1 | P | 473.3 | P | 597.7 | P | 356.5 |
| 16 | 92397_at | Cluster Incl AW123016 UI-M-BH2 1-apb-e-06-0-UI s1 Mus musculus cDNA, 3 end/clone = UI-M-BH2.1-apb-e-06-0-UI/ | 363 | P | 642.6 | P | 533.7 | A | 350.5 |
| | | clone_end = 3/gb = AW123016/gi = 6098546/ug = Mm 4077/len = 473/STRA = rev | | | | | | | |
| 16 | 92739_at | Cluster Incl L28819 Involucnn/cds = (0,1403)/gb = L28819/gi = 454418/ug = Mm.46792/len = 1404/STRA = for | 235.7 | P | 273.4 | P | 205 | P | 92.5 |
| 16 | 92894_s_at | Cluster Incl D90174 Mouse mRNA for NFI-B protein, clone NFI-B3/cds = (237,1742)/gb = D90174/gi = 220520/ug = Mm 4771/ | 198.4 | P | 226.3 | P | 248.3 | P | 94.2 |
| | | len = 1859/STRA = for | | | | | | | |
| 16 | 93421_at | Cluster Incl AF033655 PFTAIRE protein kinase 1/cds = (85,1494)/gb = AF033655/gi = 2645809/ug = Mm 6456/len = 4529/ | 108.4 | P | 165 | P | 135 | P | 113.2 |
| | | STRA = for | | | | | | | |
| 16 | 97155_at | Cluster Incl U84005 Myostatin/cds = (103,1233)/gb = U84005/gi = 2065554/ug = Mm.3514/len = 2676/STRA = for | 356.5 | P | 425.7 | P | 281.8 | P | 130.9 |
| 16 | 97874_at | Cluster Incl AI853863 UI-M-BH0-ajr-f-07-0-UI.s1 Mus musculus cDNA, 3 end/clone = UI-M-BH0-ajr-f-07-0-UI/clone_end = 3/ | 311.2 | P | 400.6 | P | 362.5 | P | 276.2 |
| | | gb = AI853863/gi = 5497769/ug = Mm 28349/len = 440/STRA = for | | | | | | | |
| 16 | 95693_at | Cluster Incl U51167 Isocitrate dehydrogenase 2/cds = (23,1594)/gb = U51167/gi = 1236983/ug = Mm 2966/len = 1863/ | 489.4 | P | 762.8 | P | 639.3 | P | 232.1 |
| | | STRA = for | | | | | | | |
| 16 | 98144_f_at | Cluster Incl AI844321 UI-M-AL1-ahm-f-07-0-UI s1 Mus musculus cDNA, 3 end/clone = UI-M-AL1-ahm-f-07-0-UI/ | 346.1 | A | 430.1 | P | 453.6 | P | 386 |
| | | clone_end = 3/gb = AI844321/gi = 5488227/ug = Mm 35650/len = 381/STRA = for | | | | | | | |
| 16 | 102029_at | Cluster Incl AF017111 Interleukin 16/cds = (168,2042)/gb = AF017111/gi = 3127042/ug = Mm 10137/len = 2876/STRA = rev | 112.6 | P | 149.3 | P | 93.3 | A | 84.5 |
| 16 | 103637_at | Cluster Incl AJ223966 N-acetyl galactosaminidase, alpha/cds = (0,1247)/gb = AJ223966/gi = 3327015/ug = Mm 20325/ | 277.5 | P | 375.2 | P | 323.6 | P | 229.5 |
| | | len = 1248/STRA = for | | | | | | | |
| 16 | 94417_at | Cluster Incl AI852949 UI-M-BH0-aiz-e-01-0-UI s1 Mus musculus cDNA, 3 end/clone = UI-M-BH0-aiz-e-01-0-UI/ | 110.4 | A | 130.9 | P | 108.6 | P | 84.3 |
| | | clone_end = 3/gb = AI852949/gi = 5496855/ug = Mm 26168/len = 338/STRA = for | | | | | | | |
| 16 | 96176_at | Cluster Incl AI847916 UI-M-AI1-afl-c-02-0-UI s1 Mus musculus cDNA, 3 end/clone = UI-M-AI1-afl-c-02-0-UI/clone_end = 3/ | 343 | A | 448.7 | P | 405.8 | P | 349.5 |
| | | gb = AI847916/gi = 5491822/ug = Mm 28409/len = 447/STRA = for | | | | | | | |
| 16 | 98449_at | Cluster Incl AB014721 Mus musculus mRNA for mSART-1(806), complete cds/cds = (34,2454)/gb = AB014721/gi = 4126468/ | 106.5 | P | 209.1 | P | 79.8 | P | 44.9 |
| | | ug = Mm 34562/len = 2513/STRA = rev | | | | | | | |
| 16 | 100943_at | Cluster Incl U75215 Mus musculus neutral amino acid transporter mASCT1 mRNA, complete cds/cds = = (111,1709)/ | 161.8 | P | 316.2 | P | 285.7 | P | 182.7 |
| | | gb = U75215/gi = 2459560/ug = Mm 6379/len = 3787/STRA = for | | | | | | | |
| 16 | 102227_g_at | Cluster Incl AF017639 Mus musculus carboxypeptidase X2 mRNA, complete cds/cds = (129,2423)/gb = AF017639/ | 122.9 | P | 160.4 | P | 113 | P | 85.3 |
| | | gi = 2921091/ug = Mm 10233/len = 2490/STRA = for | | | | | | | |
| 16 | 103502_at | Cluster Incl AA866768 ud07g06 r1 Mus musculus cDNA, 5 end/clone = IMAGE-1434490/clone_end = 5/gb = AA866768/ | 52.4 | A | 159.3 | P | 112.9 | P | 44.8 |
| | | gi = 2962213/ug = Mm 22367/len = 376/STRA = for | | | | | | | |
| 16 | 92450_at | Cluster Incl AF047339: Solute camer family 12, member 4/cds = (0,3257)/gb = AF047339/gi = 3452286/ug = Mm 4190/ | 351.2 | P | 464.6 | P | 449 | P | 369.2 |
| | | len = 3775/STRA = for | | | | | | | |
| 16 | 97784_at | Cluster Incl Y14695 Mus musculus mRNA for epidermis-type lipoxygenase-3/cds = (26,2161)/gb = Y14695/gi = 5304927/ | 211.5 | M | 444.5 | P | 560.5 | P | 208.4 |
| | | ug = Mm 41989/len = 2536/STRA = for | | | | | | | |
| 16 | 100743_at | Cluster Incl AA407663 EST01744 Mus musculus cDNA, 3 end/clone = C0013E09/clone_end = 3/gb = AA407663/ | 183.8 | P | 162 | P | 141.5 | P | 103.3 |
| | | gi = 2066659/ug = Mm 3640/len = 265/STRA = rev | | | | | | | |
| 16 | 94504_at | Cluster Incl AI838655 UI-M-AO0-aca-c-08-0-UI.s1 Mus musculus cDNA, 3 end/clone = UI-M-AO0-aca-c-08-0-UI/ | 393.8 | P | 585.3 | P | 294.3 | P | 135.2 |
| | | clone_end = 3/gb = AI838655/gi = 5472868/ug = Mm 28955/len = 343/STRA = rev | | | | | | | |
| 16 | 95642_at | Cluster Incl AI842813 UI-M-AP1-agi-g-02-0-UI.s1 Mus musculus cDNA, 3 end/clone = UI-M-AP1-agi-g-02-0-UI/ | 154.3 | P | 673.9 | P | 209.8 | P | 7.9 |
| | | clone_end = 3/gb = AI842813/gi = 5477026/ug = Mm 29492/len = 403/STRA = for | | | | | | | |
| 16 | 99156_at | Cluster Incl AI853370 UI-M-BH0-air-e-04-0-UI s1 Mus musculus cDNA, 3 end/clone = UI-M-BH0-air-e-04-0-UI/ | 78.1 | P | 123.3 | P | 137.2 | P | 39.4 |
| | | clone_end = 3/gb = AI853370/gi = 5497276/ug = Mm.41617/len = 415/STRA = for | | | | | | | |
| 16 | 101571_g_at | Cluster Incl X76066 M musculus gene for insulin like growth factor binding protein 4/cds = (0,764)/gb = X76066/gi = 416032/ | 649.5 | P | 818.1 | P | 784.4 | P | 644.1 |
| | | ug = Mm 89842/len = 765/STRA = for | | | | | | | |
| 16 | 102343_at | Cluster Incl AF110520: Mus musculus major histocompatibility complex region NG27, NG28, RPS28, NADH | 370.5 | P | 456.4 | P | 330.8 | P | 489.5 |
| | | oxidoreductase, NG29, KIFC1, Fas-binding protein, BING1, tapasin, RaIGDS-like, KE2, BING4, beta 1,3-galactosyl transferase, | | | | | | | |
| | | and RPS18 genes, complete cds; S/STRA = for | | | | | | | |
| 16 | 103017_at | Cluster Incl AI060729 ub42h10 r1 Mus musculus cDNA, 5 end/clone = IMAGE-1380451/clone_end = 5/gb = AI060729/ | 131.4 | P | 135 | P | 143.5 | P | 85.5 |
| | | gi = 3336152/ug = Mm 1565/len = 457/STRA = for | | | | | | | |
| 16 | 97964_at | Cluster Incl AW122851: UI-M-BH2 1-aoz-g-06-0-UI s1 Mus musculus cDNA, 3 end/clone = UI-M-BH2 1-aoz-g-06-0-UI/ | 149.8 | P | 248.6 | P | 153.9 | P | 30.6 |
| | | clone_end = 3/gb = AW122851/gi = 6098381/ug = Mm 30729/len = 462/STRA = for | | | | | | | |
| 16 | 100408_at | Cluster Incl AA839465-vw50d06 r1 Mus musculus cDNA, 5 end/clone = IMAGE-1247243/clone_end = 5/gb = AA839465/ | 140.2 | P | 153.7 | P | 132.5 | P | 103.1 |
| | | gi = 2915560/ug = Mm 46563/len = 437/STRA = for | | | | | | | |
| 16 | 100991_at | Cluster Incl AI852849 UI-M-BH0-ajk-d-01-0-UI s1 Mus musculus cDNA, 3 end/clone = UI-M-BH0-ajk-d-01-0-UI/ | 215.9 | A | 475.3 | P | 232.8 | P | 179.4 |
| | | clone_end = 3/gb = AI852849/gi = 5496755/ug = Mm 6818/len = 348/STRA = for | | | | | | | |
| 16 | 101901_at | Cluster Incl AB024713 Mus musculus mRNA for Supl15h, complete cds/cds = (6,749)/gb = AB024713/gi = 5103141/ | 306.7 | P | 400.6 | P | 294.7 | P | 329.6 |
| | | ug = Mm 89579/len = 1249/STRA = for | | | | | | | |
| 16 | 102969_at | Cluster Incl AI847369 UI-M-AN1-afh-g-04-0-UI s1 Mus musculus cDNA, 3 end/clone = UI-M-AN1-afh-g-04-0-UI/ | 102 | P | 175.5 | P | 97.8 | A | 115.6 |
| | | clone_end = 3/gb = AI847369/gi = 5491240/ug = Mm 183/len = 436/STRA = for | | | | | | | |
| 16 | 103273_s_at | Cluster Incl AF037312 untitled/cds = (0,2051)/gb = AF037312/gi = 3851489/ug = Mm 20868/len = 2147/STRA = for | 158.5 | A | 196.8 | P | 167.5 | P | 126.5 |
| 16 | 96577_i_at | Cluster Incl AI853019 UI-M-BH0-ajn-d-04-0-UI s1 Mus musculus cDNA, 3 end/clone = UI-M-BH0-ajn-d-04-0-UI/ | 34.5 | A | 184.8 | P | 166.3 | P | 116.9 |
| | | clone_end = 3/gb = AI853019/gi = 5496925/ug = Mm 3021/len = 250/STRA = for | | | | | | | |
| 16 | 98859_at | Cluster Incl M99054 Acid phosphatase 5, tartrate resistant/cds = (132,1115)/gb = M99054/gi = 553868/ug = Mm.5119/ | 204 | P | 204 | P | 268.1 | P | 144.7 |
| | | len = 1386/STRA = for | | | | | | | |
| 16 | 101703_at | Cluster Incl V00716 Mouse pseudogene for alpha-globin without intervening sequences (the normal alpha-globin gene has | 1471.8 | P | 2143.4 | A | 2161 | P | 1202 |
| | | two introns)/cds = (32,436)/gb = V00716/gi = 49902/ug = Mm 41983/len = 542/STRA = for | | | | | | | |
| 16 | 92558_at | Cluster Incl M84487 Vascular cell adhesion molecule 1/cds = (57,2276)/gb = M84487/gi = 202345/ug = Mm 1021/len = 3014/ | 93.5 | A | 77.8 | P | 85.4 | A | 64.4 |
| | | STRA = for | | | | | | | |
| 16 | 94861_at | Cluster Incl AA624073 vq71f12 s1 Mus musculus cDNA, 5 end/clone = IMAGE-1107791/clone_end = 5/gb = AA624073/ | 148.3 | P | 144.4 | A | 131.4 | P | 123.7 |
| | | gi = 2527949/ug = Mm.23682/len = 342/STRA = for | | | | | | | |
| 16 | 98005_at | Cluster Incl AW125442 UI-M-BH2 3-aqh-g-12-0-UI s1 Mus musculus cDNA, 3 end/clone = UI-M-BH2 3-aqh-g-12-0-UI/ | 148.7 | A | 149 | P | 170.9 | P | 93 |
| | | clone_end = 3/gb = AW125442/gi = 6100972/ug = Mm 3193/len = 487/STRA = for | | | | | | | |
| 16 | 101844_at | Cluster Incl U94700: Peroxisomal sarcosine oxidase/cds = (4,1176)/gb = U94700/gi = 2801410/ug = Mm 8543/len = 1686/ | 328.8 | P | 296.2 | P | 297 | P | 285.4 |
| | | STRA = for | | | | | | | |
| 16 | 102684_at | Cluster Incl U82375 Mus musculus MSG2alpha, beta, gamma, delta and epsilon salivary protein (Vcs2) genes, complete | 29.2 | A | 57.7 | P | 19.4 | P | 14.2 |
| | | cds/cds = (154,585)/gb = U82375/gi = 2289098/ug = Mm 1397/len = 743/STRA = for | | | | | | | |
| 16 | 92971_at | Cluster Incl AW125849 UI-M-BH2.2-aqk-d-08-0-UI.s1 Mus musculus cDNA, 3 end/clone = UI-M-BH2 2-aqk-d-08-0-UI/ | 90.4 | P | 208 | P | 148.6 | P | 75.4 |
| | | clone_end = 3/gb = AW125849/gi = 6101379/ug = Mm.5002/len = 468/STRA = rev | | | | | | | |
| 16 | 93463_at | Cluster Incl AW122517 UI-M-BH2.2-aox-a-10-0-UI s1 Mus musculus cDNA, 3 end/clone = UI-M-BH2 2-aox-a-10-0-UI/ | −77.2 | P | −122.6 | A | −59.8 | A | −96.5 |
| | | clone_end = 3/gb = AW122517/gi = 6098041/ug = Mm 69024/len = 439/STRA = rev | | | | | | | |
| 16 | 100634_at | Cluster Incl AF056194 Mus musculus short-chain dehydrogenase CRAD2 mRNA, complete cds/cds = (97,1047)/ | −11.5 | M | −66.9 | P | −100.1 | P | −212.7 |
| | | gb = AF056194/gi = 3294554/ug = Mm 6696/len = 1612/STRA = for | | | | | | | |
| 16 | 103085_at | Cluster Incl AB013095 Mus musculus mRNA for heme-binding protein, complete cds/cds = (57,629)/gb = AB013095/ | 211.1 | P | 334.7 | M | 266.1 | P | 137.2 |
| | | gi = 3724327/ug = Mm 17249/len = 1033/STRA = for | | | | | | | |
| 16 | 103804_at | Cluster Incl AB006960 Mus musculus mRECK mRNA, complete cds/cds = (6,2921)/gb = AB006960/gi = 3810870/ | 80.4 | A | 172.5 | P | 242.6 | P | 32.6 |
| | | ug = Mm 20862/len = 4094/STRA = for | | | | | | | |
| 16 | 95568_at | Cluster Incl AI853412.UI-M-BH0-ajy-a-05-0-UI s1 Mus musculus cDNA, 3 end/clone = UI-M-BH0-ajy-a-05-0-UI/ | 45.2 | P | 56.8 | P | 25 | A | 18.2 |
| | | clone_end = 3/gb = AI853412/gi = 5497318/ug = Mm 27799/len = 587/STRA = for | | | | | | | |
| 16 | 96810_at | Cluster Incl AI154017 ud58a11 r1 Mus musculus cDNA, 5 end/clone = IMAGE-1450076/clone_end = 5/gb = AI154017/ | 239.3 | A | 281.1 | P | 236.8 | P | 215.1 |
| | | gi = 3682486/ug = Mm 29266/len = 156/STRA = for | | | | | | | |
| 16 | 104402_at | Cluster Incl AA030469 mi23c07 r1 Mus musculus cDNA, 5 end/clone = IMAGE-464364/clone_end = 5/gb = AA030469/ | −5.3 | A | 120.7 | P | 68.8 | P | 94 |
| | | gi = 1497632/ug = Mm 2869/len = 342/STRA = for | | | | | | | |
| 16 | 93949_at | Cluster lncl M63658 M musculus G protein beta-subunit mRNA, complete cds/cds = (40,1062)/gb = M63658/gi = 193435/ | 62.9 | P | 54.1 | A | 105 | P | 42.3 |
| | | ug = Mm 9336/len = 1135/STRA = for | | | | | | | |
| 16 | 98829_at | Cluster Incl X80417 Potassium inwardly rectifying channel, subfamily J, member 12/cds = (1,1284)/gb = X80417/gi = 550388/ | 43.8 | A | 124.6 | P | 72.7 | A | 32.5 |
| | | ug = Mm 4970/len = 1618/STRA = for | | | | | | | |
| 16 | 102562_at | Cluster Incl Z80833 M musculus spermidine synthase pseudogene 2/cds = (0,852)/gb = Z80833/gi = 1592790/ug = Mm 57224/ | 509.8 | A | 571.5 | P | 545.8 | P | 482.4 |
| | | len = 853/STRA = for | | | | | | | |
| 16 | 95513_at | Cluster Incl AW124310 UI-M-BH2.1-apq-d-03-0-UI s1 Mus musculus cDNA, 3 end/clone = UI-M-BH2 1 -apq-d-03-0-UI/ | 139.6 | P | 151.8 | A | 194.1 | P | 120.2 |
| | | clone_end = 3/gb = AW124310/gi = 6099840/ug = Mm.25298/len = 234/STRA = for | | | | | | | |
| 16 | 95641_at | Cluster Incl AI853899 UI-M-BH0-aju-c-09-0-UI.s1 Mus musculus cDNA, 3 end/clone = UI-M-BH0-aju-c-09-0-UI/ | 403.9 | P | 818.6 | P | 388.9 | P | 306.6 |
| | | clone_end = 3/gb = AI853899/gi = 5497805/ug = Mm.29491/len = 380/STRA = for | | | | | | | |
| 16 | 103043_at | Cluster Incl Z35294 M musculus MTCP-1 gene/cds = (540,746)/gb = Z35294/gi = 603859/ug = Mm 16366/len = 954/ | 60.5 | P | 22.1 | M | 91.1 | P | 56.7 |
| | | STRA = for | | | | | | | |
| 16 | 103831_at | Cluster Incl U72194 Mus musculus muskelin mRNA, complete cds/cds = (12,2219)/gb = U72194/gi = 3493461/ | 84.7 | A | 169.1 | P | 137.4 | P | 173.7 |
| | | ug = Mm 20930/len = 3975/STRA = for | | | | | | | |
| 16 | 103869_at | Cluster Incl U16175 Mus musculus thrombospondin 3 (Thbs3) gene, partial cds and mucin 1 (Muc1) gene, complete cds/ | 140.8 | P | 169.8 | A | 145.8 | M | 142.4 |
| | | cds = (0,371)/gb = U16175/gi = 608488/ug = Mm 2114/len = 615/STRA = for | | | | | | | |
| 16 | 104042_at | Cluster Incl AI842275 UI-M-AM1-afz-d-12-0-UI s1 Mus musculus cDNA, 3 end/clone = UI-M-AM1-afz-d-12-0-UI/ | 250 | M | 229.8 | P | 254.7 | P | 197.6 |
| | | clone end = 3/gb = AI842275/gi = 5476528/ug = Mm 21470/len = 412/STRA = for | | | | | | | |
| 16 | 104752_at | Cluster Incl AB028863 Mus musculus mRNA for MMRP19, complete cds/cds = (5,730)/gb = AB028863/gi = 5103282/ | 324.2 | A | 499 | P | 482.7 | P | 192.3 |
| | | ug = Mm.24772/len = 840/STRA = for | | | | | | | |
| 16 | 94940_at | Cluster Incl AW123316 UI-M-BH2 1-apf-b-01-0-UI s1 Mus musculus cDNA, 3 end/clone = UI-M-BH2 1-apf-b-01-0-UI/ | 181.2 | P | 155.5 | A | 119.4 | P | 81.8 |
| | | clone_end = 3/gb = AW123316/gi = 6098846/ug = Mm.26925/len = 414/STRA = for | | | | | | | |
| 16 | 100968_at | Cluster Incl AW122609.UI-M-BH2 2-aol-a-11-0-UI s1 Mus musculus cDNA, 3 end/clone = UI-M-BH2 2-aol-a-11-0-UI/ | 0.4 | A | 133.3 | P | 82.7 | A | 87 |
| | | clone_end = 3/gb = AW122609/gi = 6098136/ug = Mm.6626/len = 257/STRA = rev | | | | | | | |
| 16 | 101939_at | Cluster Incl AI849344: UI-M-AJ1-aha-f-04-0-UI.s1 Mus musculus cDNA, 3 end/clone = UI-M-AJ1-aha-f-04-0-UI/ | 667 | P | 808.8 | A | 634.4 | A | 396.9 |
| | | clone_end = 3/gb = AI849344/gi = 5493250/ug = Mm.95187/len = 570/STRA = for | | | | | | | |
| 16 | 101940_at | Cluster Incl AJ222800: Mus musculus mRNA for neutral sphingomyelinase/cds = (309,1568)/gb = AJ222800/gi = 3021479/ | 226 | P | 227.7 | A | 259 | P | 126 |
| | | ug = Mm 953/len = 1627/STRA = for | | | | | | | |
| 16 | 103256_at | Cluster Incl AI509811: vx28b11 y1 Mus musculus cDNA, 5 end/clone = IMAGE-1265757/clone_end = 5/gb = AI509811/ | 154.8 | A | 190 | A | 194.5 | P | 170.2 |
| | | gi = 4408716/ug = Mm 2072/len = 251/STRA = for | | | | | | | |
| 16 | 92314_at | Cluster Incl U25652: Procollagen, type XII, alpha 1/cds = (83,9286)/gb = U25652/gi = 953236/ug = Mm 3819/len = 9704/ | 162.6 | A | 133.2 | M | 181.8 | P | 67.6 |
| | | STRA = rev | | | | | | | |
| 16 | 92482_at | Cluster Incl AF006466: Mus musculus lymphocyte specific formin related protein (Fr1) mRNA, complete cds/cds = (0,3194)/ | 194.1 | A | 318.7 | A | 262.1 | A | 163.4 |
| | | gb = AF006466/gi = 4101719/ug = Mm 42152/len = 3195/STRA = for | | | | | | | |
| 16 | 96509_at | Cluster Incl J05019 Fc receptor, IgE, high affinity I, beta polypeptide/cds = (22,729)/gb = J05019/gi = 193238/ug = Mm 2544/ | 153.1 | P | 210.7 | P | 148.3 | M | 127.5 |
| | | len = 1010/STRA = for | | | | | | | |
| 17 | AFFX-BioB- | E Coli BioB middle | 467.6 | P | 676.4 | P | 460.1 | P | 454.8 |
| | M_at | | | | | | | | |
| 17 | AFFX-BioC- | E Coli BioC 5 prime | 1092.2 | P | 1369.3 | P | 1028.1 | P | 1026.3 |
| | 5_at | | | | | | | | |
| 17 | AFFX-BioDn- | E Coli BioD 5 prime | 1749.7 | P | 2041 | P | 1646.6 | P | 1755.4 |
| | 5_at | | | | | | | | |
| 17 | 92554_at | Cluster Incl AF059735 Mus musculus C-terminal binding protein 2 CtBP2 mRNA, complete cds/cds = (0,1337)/ | 459.1 | P | 518.1 | P | 378.3 | P | 446.8 |
| | | gb = AF059735/gi = 3513570/ug = Mm.10172/len = 1338/STRA = rev | | | | | | | |
| 17 | 92593_at | Cluster Incl D13664 Mus musculus osf-2 mRNA for osteoblast specific factor 2, complete cds/cds = (18,2453)/gb = D13664/ | 4415.6 | P | 4847.7 | P | 3352.3 | P | 3865.5 |
| | | gi = 393321/ug = Mm 10681/len = 3187/STRA = for | | | | | | | |
| 17 | 92771_at | Cluster Incl AB013357 Mus musculus mRNA for 49 kDa zinc finger protein, complete cds/cds = (110,1492)/gb = AB013357/ | 1674.2 | P | 1796.7 | P | 1173.8 | P | 1653.1 |
| | | gi = 4153885/ug = Mm 12236/len = 2082/STRA = for | | | | | | | |
| 17 | 93067_f_at | Cluster Incl U62674 Mus musculus histone H2a 2-615 (H2a-615), and histone H3 2-615 (H3-615) genes, complete cds/ | 959.5 | P | 1083.5 | P | 827.9 | P | 932.6 |
| | | cds = (43,435)/gb = U62674/gi = 1458142/ug = Mm.15686/len = 515/STRA = for | | | | | | | |
| 17 | 93070_at | Cluster Incl AI847564 UI-M-AM1-afv-g-08-0-UI s1 Mus musculus cDNA, 3 end/clone = UI-M-AM1-afv-g-08-0-UI/ | 470.2 | P | 483.1 | P | 411.8 | P | 530.7 |
| | | clone_end = 3/gb = AI847564/gi = 5491470/ug = Mm 15701/len = 348/STRA = for | | | | | | | |
| 17 | 93097_at | Cluster Incl U51805 Mus musculus liver arginase mRNA, complete cds/cds = (141,1112)/gb = U51805/gi = 1293092/ | 269.5 | P | 175.3 | P | 155.5 | P | 236.2 |
| | | ug = Mm 16423/len = 1221/STRA = for | | | | | | | |
| 17 | 93266_at | Cluster Incl U04541 Mus musculus alpha-tropomyosin slow mRNA, complete cds/cds = (53,910)/gb = U04541/gi = 439845/ | 1216.7 | P | 1110.2 | P | 1153.2 | P | 828.7 |
| | | ug = Mm 17306/len = 1091/STRA = for | | | | | | | |
| 17 | 93325_at | Cluster Incl AI845735 UI-M-AQ1-aeb-b-07-0-UI s1 Mus musculus cDNA, 3 end/clone = UI-M-AQ1-aeb-b-07-0-UI/ | 300.8 | P | 361.8 | P | 222.3 | P | 336.4 |
| | | clone_end = 3/gb = AI845735/gi = 5489641/ug = Mm 18579/len = 362/STRA = for | | | | | | | |
| 17 | 93588_at | Cluster Incl Z54179 M musculus mRNA (evolutionary conserved transcript)/cds = (221,802)/gb = Z54179/gi = 1655636/ | 197.7 | P | 196.8 | P | 154.9 | P | 164 |
| | | ug = Mm 2080/len = 1217/STRA = for | | | | | | | |
| 17 | 93752_at | Cluster lncl AI848393: UI-M-AH1-ago-g-10-0-UI s1 Mus musculus cDNA, 3 end/clone = UI-M-AH1-ago-g-10-0-UI/ | 596.8 | P | 589.3 | P | 515.8 | P | 588.6 |
| | | clone_end = 3/gb = AI848393/gi = 5492294/ug = Mm 21118/len = 468/STRA = for | | | | | | | |
| 17 | 93768_f_at | Cluster Incl AW124052: UI-M-BH2 1-apn-e-06-0-UI s1 Mus musculus cDNA, 3 end/clone = UI-M-BH2 1-apn-e-06-0-UI/ | 762.3 | P | 724.7 | P | 582.4 | P | 638.5 |
| | | clone_end = 3/gb = AW124052/gi = 6099582/ug = Mm 21183/len = 246/STRA = for | | | | | | | |
| 17 | 93801 _at | Cluster Incl AA763275 vv89d02 r1 Mus musculus cDNA, 5 end/clone = IMAGE-1229571/clone_end = 5/gb = AA763275/ | 3778.2 | P | 3704.9 | P | 2426.2 | P | 3875.3 |
| | | gi = 2813022/ug = Mm 21408/len = 357/STRA = for | | | | | | | |
| 17 | 94044_at | Cluster Incl AA692708 vr54d07 s1 Mus musculus cDNA, 5 end/clone = IMAGE-1124461/clone_end = 5/gb = AA692708/ | 430.7 | P | 342 | P | 214.7 | P | 359.8 |
| | | gi = 2693646/ug = Mm 21964/len = 170/STRA = for | | | | | | | |
| 17 | 94232_at | Cluster Incl AI849928: UI-M-BG0-ahz-e-10-0-UI s1 Mus musculus cDNA, 3 end/clone = UI-M-BG0-ahz-e-10-0-UI/ | 1262.5 | P | 1008.7 | P | 924.6 | P | 1123.7 |
| | | clone_end = 3/gb = AI849928/gi = 5493834/ug = Mm 22288/len = 482/STRA = for | | | | | | | |
| 17 | 94295_at | Cluster Incl AF017085 Mus musculus BAP-135 homolog (Diws1t) mRNA, complete cds/cds = (87,3023)/gb = AF017085/ | 1203.9 | P | 1157 | P | 1064.3 | P | 844.3 |
| | | gi = 2653820/ug = Mm 22593/len = 4091/STRA = for | | | | | | | |
| 17 | 94296_s_at | Cluster Incl AF043220 Mus musculus TFII-I protein short form mRNA, alternatively spliced, complete cds/cds = (4,2880)/ | 303.9 | P | 185.5 | P | 224.4 | P | 214.8 |
| | | gb = AF043220/gi = 2895589/ug = Mm 22593/len = 2936/STRA = for | | | | | | | |
| 17 | 96041_at | Cluster Incl AB016424 Mus musculus rbm3 mRNA, complete cds/cds = (85,546)/gb = AB016424/gi = 3810891/ug = Mm 2591/ | 6019.1 | P | 6011.7 | P | 4822.8 | P | 6221.2 |
| | | len = 1158/STRA = for | | | | | | | |
| 17 | 96104_at | Cluster Incl AI047107 uh58b11 r1 Mus musculus cDNA, 5 end/clone = IMAGE-1749597/clone_end = 5/gb = AI047107/ | 366.4 | P | 403.4 | P | 259.3 | P | 368.7 |
| | | gi = 3295394/ug = Mm 26879/len = 403/STRA = for | | | | | | | |
| 17 | 96649_at | Cluster Incl AW046552 UI-M-BH1-alb-b-06-0-UI.s1 Mus musculus cDNA, 3 end/clone = UI-M-BH1-alb-b-06-0-UI/ | 370.4 | P | 450.6 | P | 306.6 | P | 319.4 |
| | | clone_end = 3/gb = AW046552/gi = 5907081/ug = Mm 27328/len = 517/STRA = for | | | | | | | |
| 17 | 97857_at | Cluster Incl AW047762 UI-M-BH1-alk-d-04-0-UI s1 Mus musculus cDNA, 3 end/clone = UI-M-BH1-alk-d-04-0-UI/ | 253.5 | P | 321.1 | P | 292.2 | P | 326.4 |
| | | clone_end = 3/gb = AW047762/gi = 5908291/ug = Mm 28300/len = 387/STRA = for | | | | | | | |
| 17 | 97888_at | Cluster Incl AI849676 UI-M-AL1-ahl-e-06-0-UI.s1 Mus musculus cDNA, 3 end/clone = UI-M-AL1-ahl-e-06-0-UI/ | 397.2 | P | 317.6 | P | 230.3 | P | 365 |
| | | clone_end = 3/gb = AI849676/gi = 5493582/ug = Mm.28397/len = 367/STRA = for | | | | | | | |
| 17 | 97893_at | Cluster Incl AB017697 Mus musculus TLP21 mRNA for 21-kDa TBP-like protein, complete cds/cds = (272,832)/ | 126.5 | P | 128.7 | P | 108.1 | P | 111.3 |
| | | gi = AB017697/gi = 4218069/ug = Mm 28415/len = 1273/STRA = for | | | | | | | |
| 17 | 97908_at | Cluster Incl AW120676 UI-M-BH2 3-anz-b-10-0-UI.s1 Mus musculus cDNA, 3 end/clone = UI-M-BH2.3-anz-b-10-0-UI/ | 1205.3 | P | 1310.7 | P | 1179.4 | P | 954.1 |
| | | clone_end = 3/gb = AW120676/gi = 6095974/ug = Mm.26474/len = 452/STRA = for | | | | | | | |
| 17 | 98924_at | Cluster Incl Y08027 M musculus ART3 gene/cds = (0,1112)/gb = Y08027/gi = 2677615/ug = Mm 28637/len = 1113/STRA = for | 128.1 | P | 198.9 | P | 146 | P | 160.4 |
| 17 | 94537_at | Cluster Incl AI838859 UI-M-AN0-ack-c-06-0-UI s1 Mus musculus cDNA, 3 end/clone = UI-M-AN0-ack-c-06-0-UI/ | 1859.5 | P | 2074.4 | P | 1574.1 | P | 1419.4 |
| | | clone_end = 3/gb = AI838859/gi = 5473072/ug = Mm 29057/len = 349/STRA = for | | | | | | | |
| 17 | 94542_at | Cluster Incl AA929348: vz41b07.r1 Mus musculus cDNA, 5 end/clone = IMAGE-1329013/clone_end = 5/gb = AA929348/ | 228.1 | P | 209.6 | P | 147.2 | P | 224.8 |
| | | gi = 3078657/ug = Mm 29087/len = 166/STRA = for | | | | | | | |
| 17 | 95056_r_at | Cluster Incl AW122747 UI-M-BH2.2-aot-g-01-0-UI s1 Mus musculus cDNA, 3 end/clone = UI-M-BH2 2-aot-g-01-0-UI/ | 200.8 | P | 159.2 | P | 128.3 | P | 113 |
| | | clone_end = 3/gb = AW122747/gi = 6098277/ug = Mm 29150/len = 468/STRA = for | | | | | | | |
| 17 | 95078_at | Cluster Incl AI841400 UI-M-AH0-acx-a-06-0-UI.s1 Mus musculus cDNA, 3 end/clone = UI-M-AH0-acx-a-06-0-UI/ | 300.7 | P | 291.5 | P | 239.5 | P | 190.8 |
| | | clone_end = 3/gb = AI841400/gi = 5475613/ug = Mm 29213/len = 213/STRA = for | | | | | | | |
| 17 | 95092_at | Cluster Incl J05479: Mouse calcineurin catalytic subunit mRNA, complete cds/cds = (75,1640)/gb = J05479/gi = 192347/ | 485.3 | P | 580.6 | P | 443.4 | P | 576.7 |
| | | ug = Mm.293/len = 2194/STRA = for | | | | | | | |
| 17 | 95142_s_at | Cluster lncl U10407 Capping protein beta 1/cds = (18,836)/gb = U10407/gi = 500748/ug = Mm 2945/len = 1014/STRA = for | 1200.9 | P | 1561.8 | P | 1075.7 | P | 1494.4 |
| 17 | 95646_at | Cluster Incl U01170 Camiline palmitoyltransferase 2/cds = (0,1976)/gb = U01170/gi = 425266/ug = Mm 29499/len = 1977/ | 961.9 | P | 686.9 | P | 563.8 | P | 547.5 |
| | | STRA = for | | | | | | | |
| 17 | 95730_at | Cluster Incl AI840884 UI-M-AM0-adm-b-06-0-UI s1 Mus musculus cDNA, 3 end/clone = UI-M-AM0-adm-b-06-0-UI/ | 831.9 | P | 907.9 | P | 677.6 | P | 703.4 |
| | | clone_end = 3/gb = AI840884/gi = 5475097/ug = Mm 29741/len = 317/STRA = for | | | | | | | |
| 17 | 96299_at | Cluster Incl AIB47298: UI-M-AN1-afh-c-08-0-UI s1 Mus musculus cDNA, 3 end/clone = UI-M-AN1-afh-c-08-0-UI/ | 1954 | P | 2050.9 | P | 1220.5 | P | 1903 |
| | | clone_end = 3/gb = AI847298/gi = 5491204/ug = Mm 29909/len = 328/STRA = for | | | | | | | |
| 17 | 96322_at | Cluster Incl AI836001 UI-M-AJ0-abb-b-07-0-UI s1 Mus musculus cDNA, 3 end/clone = UI-M-AJ0-abb-b-07-0-UI/ | 673.1 | P | 837.4 | P | 698.7 | P | 727.3 |
| | | clone_end = 3/gb = AI836001/gi = 5470254/ug = Mm 29945/len = 481/STRA = for | | | | | | | |
| 17 | 97518_at | Cluster Incl D29016 Mouse mRNA for squalene synthase/cds = (20,1270)/gb = D29016/gi = 468456/ug = Mm 3204/len = 1634/ | 759 | P | 972.3 | P | 469 | P | 859.3 |
| | | STRA/ = for | | | | | | | |
| 17 | 98039_at | Cluster Incl AI853819 UI-M-BH0-ajr-a-08-0-UI s1 Mus musculus cDNA, 3 end/clone = UI-M-BH0-ajr-a-08-0-UI/ | 620.2 | P | 875 | P | 728 | P | 741.2 |
| | | clone_end = 3/gb = AI853819/gi = 5497725/ug = Mm 34002/len = 369/STRA = for | | | | | | | |
| 17 | 98138_at | Cluster Incl AI854776 UI-M-BH0-aka-d-05-0-UI s1 Mus musculus cDNA, 3 end/clone = UI-M-BH0-aka-d-05-0-UI/ | 396.4 | P | 345.3 | P | 253.9 | P | 330.7 |
| | | clone_end = 3/gb = AI854776/gi = 5498682/ug = Mm.35538/len = 333/STRA = for | | | | | | | |
| 17 | 98604_at | Cluster Incl U79774 Mus musculus NNP-1 var (NNP-1) mRNA, complete cds/cds = (43,1527)/gb = U79774/gi = 2258277/ | 438.7 | P | 487.5 | P | 441.7 | P | 385 |
| | | ug = Mm 38344/len = 1946/STRA = for | | | | | | | |
| 17 | 99102_at | Cluster Incl U67874.Ubiquitin specific protease 9, X chromosome/cds = (355,8034)/gb = U67874/gi = 1527198/ug = Mm 396/ | 88.3 | P | 81 | P | 72.7 | P | 66.1 |
| | | len = 10323/STRA = for | | | | | | | |
| 17 | 99121_at | Cluster Incl X90875 M musculus mRNA for FXR1 protein/cds = (13,1632)/gb = X90875/gi = 1122421/ug = Mm 4038/ | 238.5 | P | 181.9 | P | 170.1 | P | 153.8 |
| | | len = 2060/STRA = for | | | | | | | |
| 17 | 99148_at | Cluster Incl AI852862 UI-M-BH0-ajk-e-02-0-UI s1 Mus musculus cDNA, 3 end/clone = UI-M-BH0-ajk-e-02-0-UI/ | 1225.8 | P | 938.8 | P | 1084 | P | 847.4 |
| | | clone_end = 3/gb = AI852862/gi = 5496768/ug = Mm.41502/len = 266/STRA = for | | | | | | | |
| 17 | 99184_at | Cluster Incl AW120896 UI-M-BH2.3-aob-b-10-0-UI s1 Mus musculus cDNA, 3 end/clone = UI-M-BH2 3-aob-b-10-0-UI/ | 445.4 | P | 799.5 | P | 556.6 | P | 476.1 |
| | | clone_end = 3/gb = AW120896/gi = 6096229/ug = Mm 41853/len = 302/STRA = for | | | | | | | |
| 17 | 99580_s_at | Cluster Incl U16818 Mus musculus UDP glucuronosyltransferase (UGT1-06) mRNA, complete cds/cds = (128,1723)/ | 1140.9 | P | 1377.8 | P | 1135 | P | 1188.5 |
| | | gb = U16818/gi = 801898/ug = Mm 42472/len = 2300/STRA = for | | | | | | | |
| 17 | 99582_at | Cluster Incl M76124 Mus musculus EGP314 precursor mRNA, complete cds/cds = (107,1051)/gb = M76124/gi = 193007/ | 117.4 | P | 90 | P | 88.8 | P | 140.3 |
| | | ug = Mm 4259/len = 1710/STRA = for | | | | | | | |
| 17 | 99666_at | Cluster Incl AW125431: UI-M-BH2.3-aqh-g-01-0-UI s1 Mus musculus cDNA, 3 end/clone = UI-M-BH2 3-aqh-g-01-0-UI/ | 2272.1 | P | 2549.7 | P | 2222.6 | P | 2389.6 |
| | | clone_end = 3/gb = AW125431/gi = 6100961/ug = Mm 43822/len = 406/STRA = for | | | | | | | |
| 17 | 100587_f_at | Cluster Incl AI843959 UI-M-AP1-agh-e-01-0-UI s1 Mus musculus cDNA, 3 end/clone = UI-M-AP1-agh-e-01-0-UI/ | 251 | P | 325.5 | P | 184.2 | P | 211.7 |
| | | clone_end = 3/gb = AI843959/gi = 5478172/ug = Mm 622/len = 379/STRA = for | | | | | | | |
| 17 | 101557_at | Cluster Incl AF043070 Mus musculus branched chain alpha ketoacid dehydrogenase kinase (bckd-kinase) mRNA, nuclear | 979.3 | P | 1112.8 | P | 977.2 | P | 1180.8 |
| | | gene encoding mitochondrial protein, complete cds/cds = (106,1344)/gb = AF043070/gi = 2809219/ug = Mm.8903/len = 1654/ | | | | | | | |
| | | STRA = for | | | | | | | |
| 17 | 101564_at | Clusler Incl U21855 Mus musculus mCAF1 protein mRNA, complete cds/cds = (39,896)/gb = U21855/gi = 726135/ | 191.7 | P | 117.5 | P | 112.5 | P | 109.1 |
| | | ug = Mm 89351/len = 1264/STRA = for | | | | | | | |
| 17 | 101962_at | Cluser Incl AI854884: UI-M-BH0-akb-f-11-0-UI s1 Mus musculus cDNA, 3 end/clone = UI-M-BH0-akb-f-11-0-UI/ | 1255.7 | P | 1297.7 | P | 1173.1 | P | 928.6 |
| | | clone_end = 3/gb = AI854884/gi = 5498790/ug = Mm 92820/len = 407/STRA = for | | | | | | | |
| 17 | 101990_at | Cluster Incl X51905: Lactate dehydrogenase 2, B chain/cds = (46,1050)/gb = X51905/gi = 52879/ug = Mm 9745/len = 1238/ | 397.6 | P | 317.4 | P | 262.5 | P | 305 |
| | | STRA = for | | | | | | | |
| 17 | 102018_at | Cluster Incl AI854879 UI-M-BH0-akb-f-05-0-UI.s1 Mus musculus cDNA, 3 end/clone = UI-M-BH0-akb-f-05-0-UI/ | 322.7 | P | 406.3 | P | 273.3 | P | 299.6 |
| | | clone_end = 3/gb = AI854879/gi = 5498785/ug = Mm 10038/len = 127/STRA = for | | | | | | | |
| 17 | 102054_at | Cluster Incl AB011370.Mus musculus Ankhzn mRNA, complete cds/cds = (21,3575)/gb = AB011370/gi = 2914016/ | 378.2 | P | 102.1 | P | 326.1 | P | 215.8 |
| | | ug = Mm 10313/len = 7143/STRA = for | | | | | | | |
| 17 | 102060_at | Cluster Incl AF051357 Mus musculus golgin-245 (olp-1) mRNA, complete cds/cds = (87,3743)/gb = AF051357/gi = 2952521/ | 456.5 | P | 460.8 | P | 364 | P | 308.7 |
| | | ug = Mm 10409/len = 4393/STRA = rev | | | | | | | |
| 17 | 102812_I_at | Cluster Incl AW210346 uI53g12 y1 Mus musculus cDNA, 5 end/clone = IMAGE-2102086/clone_end = 5/gb = AW210346/ | 473.3 | P | 448.5 | P | 396.4 | P | 409.8 |
| | | gi = 6516286/ug = Mm.14253/len = 431/STRA = for | | | | | | | |
| 17 | 103352_at | Cluster Incl X65603 Dolichyl-phosphate alpha-N-acetylglucosaminephosphotransferase 2/cds = (59,1291)/gb = X65603/ | 279.8 | P | 390.7 | P | 219.5 | P | 276.5 |
| | | gi = 51077/ug = Mm 18353/len = 1764/STRA = for | | | | | | | |
| 17 | 103421_at | Cluster Incl D50464.Mouse SDR2 mRNA, complete cds/cds = (128,1906)/gb = D50464/gi = 1747305/ug = Mm.18910/ | 234.8 | P | 217.2 | P | 161.1 | P | 168.4 |
| | | len = 2394/STRA = for | | | | | | | |
| 17 | 103424_at | Cluster Incl AI844839 UI-M-BG0-ahv-a-06-0-UI s1 Mus musculus cDNA, 3 end/clone = UI-M-BG0-ahv-a-06-0-UI/ | 413.3 | P | 427.2 | P | 242.5 | P | 557.1 |
| | | clone_end = 3/gb = AI844839/gi = 5488785/ug = Mm 18957/len = 423/STRA = for | | | | | | | |
| 17 | 103429_i_at | Cluster Incl AW125330 UI-M-BH2 1-apy-e-12-0-UI s1 Mus musculus cDNA, 3 end/clone = UI-M-BH2 1-apy-e-12-0-UI/ | 138.9 | P | 107.2 | P | 110.3 | P | 143.4 |
| | | clone_end = 3/gb = AW125330/gi = 6100855/ug = Mm 19006/len = 208/STRA = for | | | | | | | |
| 17 | 103567_at | Cluster Incl AW123810 UI-M-BH2 1-apm-f-05-0-UI s1 Mus musculus cDNA, 3 end/clone = UI-M-BH2.1-apm-f-05-0-UI/ | 639.1 | P | 643.2 | P | 416.2 | P | 562.8 |
| | | clone_end = 3/gb = AW123810/gi = 6099340/ug = Mm 1921/len = 404/STRA = for | | | | | | | |
| 17 | 103585_at | Cluster Incl AI851408 UI-M-BH0-akl-b-03-0-UI s1 Mus musculus cDNA, 3 end/clone = UI-M-BH0-alq-b-03-0-UI/ | 202.1 | P | 195.6 | P | 172 | P | 119.2 |
| | | clone_end = 3/gb = AI851408/gi = 5495314/ug = Mm 19804/len = 342/STRA = for | | | | | | | |
| 17 | 103648_at | Cluster Incl Y08830 M musculus trop2 gene/cds = (0,953)/gb = Y08830/gi = 2764995/ug = Mm.20410/len = 954/STRA = for | 1758.3 | P | 1453.4 | P | 1440.2 | P | 1337.2 |
| 17 | 103672_at | Cluster Incl AW122572 UI-M-BH2 2-aox-f-06-0-UI s1 Mus musculus cDNA, 3 end/clone = UI-M-BH2 2-aox-f-06-0-UI/ | 462 | P | 473.9 | P | 256.4 | P | 467.1 |
| | | clone_end = 3/gb = AW122572/gi = 6098097/ug = Mm20805/len = 505/STRA = for | | | | | | | |
| 17 | 103845_at | Cluster Incl AI839005 UI-M-AN0-acm-c-06-0-UI s1 Mus musculus cDNA, 3 end/clone = UI-M-AN0-acm-c-06-0-UI/ | 484.7 | P | 515.6 | P | 340 | P | 424 |
| | | clone_end = 3/gb = AI839005/gi = 5473218/ug = Mm 2097/len = 447/STRA = rev | | | | | | | |
| 17 | 103912_at | Cluster Incl AW046563: UI-M-BH1-alb-c-08-0-UI.s1 Mus musculus cDNA, 3 end/clone = UI-M-BH1-alb-c-08-0-UI/ | 496.4 | P | 507.7 | P | 346.9 | P | 437.6 |
| | | clone_end = 3/gb = AW046563/gi = 5907092/ug = Mm.21428/len = 520/STRA = for | | | | | | | |
| 17 | 104050_at | Cluster Incl AI846060: UI-M-AK1-aew-g-02-0-UI s1 Mus musculus cDNA, 3 end/clone = UI-M-AK1-aew-g-02-0-UI/ | 1035.1 | P | 850.6 | P | 691.5 | P | 845.4 |
| | | clone_end = 3/gb = AI846060/gi = 5490006/ug = Mm 21515/len = 229/STRA = rev | | | | | | | |
| 17 | 104125_at | Cluster Incl AA763673 vp09g09 r1 Mus musculus cDNA, 5 end/clone = IMAGE-1068160/clone_end = 5/gb = AA763673/ | 841.4 | P | 549.1 | P | 557.8 | P | 573.3 |
| | | gi = 2813755/ug = Mm 22030/len = 568/STRA = for | | | | | | | |
| 17 | 104337_f_at | Cluster Incl AW208938 uo65g07 x1 Mus musculus cDNA, 3 end/clone = IMAGE-2647452/clone_end = 3/gb = AW208938/ | 333.3 | P | 293.6 | P | 231.9 | P | 259.2 |
| | | gi = 6514878/ug = Mm 2252/len = 715/STRA = for | | | | | | | |
| 17 | 104340_at | Cluster Incl AF072240 Mus musculus methyl-CpG binding protein MBD1(Mbd1) mRNA, complete cds/cds = (175,2085)/ | 170.3 | P | 138.7 | P | 134.5 | P | 101.7 |
| | | gb = AF072240/gi = 3800788/ug = Mm 22522/len = 2820/STRA = for | | | | | | | |
| 17 | 104522_at | Cluster Incl AI047617: Uh63h03.r1 Mus musculus cDNA, 5 end/clone = IMAGE-1750133/clone_end = 5/gb = AI047617/ | 334.9 | P | 138.8 | P | 200.6 | P | 227.8 |
| | | gi = 3295904/ug = Mm 22930/len = 344/STRA = for | | | | | | | |
| 17 | 104557_at | Cluster Incl AI847097 UI-M-AP1-agn-f-01-0-UI s1 Mus musculus cDNA, 3 end/clone = UI-M-AP1-agn-f-01-0-UI/ | 689.1 | P | 692.7 | P | 582 | P | 703.3 |
| | | clone_end = 3/gb = AI847097/gi = 5491003/ug = Mm 23807/len = 203/STRA = rev | | | | | | | |
| 17 | 104577_at | Cluster Incl AA920621 vz07106 r1 Mus musculus cDNA, 5 end/clone = IMAGE-1315043/clone_end = 5/gb = AA920621/ | 113.6 | P | 158.3 | P | 85.3 | P | 106.5 |
| | | gi = 3067400/ug = Mm 23949/len = 294/STRA = for | | | | | | | |
| 17 | 104582_g_at | Cluster Incl AI845438 UI-M-AO1-aej-d-10-0-UI s1 Mus musculus cDNA, 3 end/clone = UI-M-AO1-aej-d-10-0-UI/ | 197.4 | P | 224.5 | P | 144.6 | P | 153.9 |
| | | clone_end = 3/gb = AI845438/gi = 5489339/ug = Mm 23975/len = 369/STRA = for | | | | | | | |
| 17 | 104589_at | Cluster Incl AF091096 Mus musculus NNX3 mRNA, complete cds/cds = (76,1449)/gb = AF091096/gi = 4165096/ | 876.8 | P | 935.6 | P | 761.3 | P | 741.7 |
| | | ug = Mm 23997/len = 2044/STRA = for | | | | | | | |
| 17 | 104604_at | Cluster Incl U62908 Mus musculus zinc linger protein 96 (Zfp96) mRNA, complete cds/cds = (432,1937)/gb = U62908/ | 110.4 | P | 95.3 | P | 95.5 | P | 87.8 |
| | | gi = 4097500/ug = Mm.24124/len = 2307/STRA = for | | | | | | | |
| 17 | 104611_at | Cluster Incl AI854008 UI-M-BH0-arv-h-12-0-UI s1 Mus musculus cDNA, 3 end/clone = UI-M-BH0-arv-h-12-0-UI/ | 326.6 | P | 311.2 | P | 322.7 | P | 348.7 |
| | | clone_end = 3/gb = AI854008/gi = 5497914/ug = Mm 24158/len = 494/STRA = for | | | | | | | |
| 17 | 94352_at | Cluster Incl AI850675 UI-M-BG1-ail-a-01-0-UI s1 Mus musculus cDNA, 3 end/clone = UI-M-BG1-ail-a-01-0-UI/ | 362.6 | P | 441.8 | P | 221.1 | P | 347.9 |
| | | clone_end = 3/gb = AI850675/gi = 5494581/ug = Mm 25200/len = 215/STRA = for | | | | | | | |
| 17 | 94426_at | Cluster Incl AI851052 UI-M-BH0-ajv-g-02-0-UI s1 Mus musculus cDNA, 3 end/clone = UI-M-BH0-ajv-g-02-0-UI/ | 711.1 | P | 717.5 | P | 486.3 | P | 507.9 |
| | | clone_end = 3/gb = AI851052/gi = 5494958/ug = Mm.26392/len = 404/STRA = for | | | | | | | |
| 17 | 95528_at | Cluster Incl AA791234 vv92e10 r1 Mus musculus cDNA, 5 end/clone = IMAGE-1229898/clone_end = 5/gb = AA791234/ | 904.4 | P | 602.3 | P | 346.4 | P | 782 |
| | | gi = 2851354/ug = Mm 27613/len = 347/STRA = for | | | | | | | |
| 17 | 95571_at | Cluster Incl AF004100: Zinc transporter 4/cds = (0,600)/gb = AF004100/gi = 2580599/ug = Mm 27801/len = 620/STRA = rev | 177.6 | P | 181.4 | P | 299.1 | P | 97.3 |
| 17 | 95577_at | Custer Incl AW049625 UI-M-BH1-and-e-07-0-UI s1 Mus musculus cDNA, 3 end/clone = UI-M-BH1-and-e-07-0-UI/ | 479.4 | P | 478.1 | P | 369.8 | P | 486.7 |
| | | clone_end = 3/gb = AW049625/gi = 5910154/ug = Mm 27829/len = 352/STRA = rev | | | | | | | |
| 17 | 95616_at | Cluster Incl AA674714 vm74b12 s1 Mus musculus cDNA, 5 end/clone = IMAGE-1003967/clone_end = 5/gb = AA674714/ | 117.6 | P | 99.8 | P | 64.2 | P | 81.1 |
| | | gi = 2651951/ug = Mm 28020/len = 156/STRA = for | | | | | | | |
| 17 | 95637_at | Cluster Incl AI838592.UI-M-AO0-abz-e-02-0-UI s1 Mus musculus cDNA, 3 end/clone = UI-M-AO0-abz-e-02-0-UI/ | 420.8 | P | 434.3 | P | 375.8 | P | 502.1 |
| | | clone_end = 3/gb = AI838592/gi = 5472805/ug = Mm 28095/len = 321/STRA = for | | | | | | | |
| 17 | 96144_at | Cluster Incl AJ001972 Mus musculus Id4 dominant negative helix-loop-helix gene/cds = (0,485)/gb = AJ001972/gi = 2546999/ | 636.4 | P | 373.7 | P | 402.1 | P | 428.2 |
| | | ug = Mm 28223/len = 486/STRA = for | | | | | | | |
| 17 | 96151_at | Cluster Incl AA839813 vw52c02 r1 Mus musculus cDNA, 5 end/clone = IMAGE-1247426/clone_end = 5/gb = AA839813/ | 291.7 | P | 277.9 | P | 254.4 | P | 255.7 |
| | | gi = 2916407/ug = Mm 28252/len = 527/STRA = for | | | | | | | |
| 17 | 96166_at | Cluster Incl AI842524 UI-M-AQ1-aea-c-06-0-UI s1 Mus musculus cDNA, 3 end/clone = UI-M-AQ1-aea-c-06-0-UI/ | 518.2 | P | 748.5 | P | 520.3 | P | 592.6 |
| | | clone_end = 3/gb = AI842524/gi = 5476737/ug = Mm 28371/len = 440/STRA = for | | | | | | | |
| 17 | 96217_at | Cluster Incl AW060552: UI-M-BH1-ann-g-10-0-UI.s1 Mus musculus cDNA, 3 end/clone = UI-M-BH1-ann-g-10-0-UI/ | 276.4 | P | 255.5 | P | 150.3 | P | 183.7 |
| | | clone_end = 3/gb = AW060552/gi = 6008303/ug = Mm 28684/len = 334/STRA = for | | | | | | | |
| 17 | 96724_r_at | Cluster Incl AW122911 UI-M-BH2 1-apa-f-09-0-UI.s1 Mus musculus cDNA, 3 end/clone = UI-M-BH2 1-apa-f-09-0-UI/ | 490.1 | P | 552 | P | 456.5 | P | 386.9 |
| | | clone_end = 3/gb = AW122911/gi = 6098441/ug = Mm 28829/len = 366/STRA = for | | | | | | | |
| 17 | 96734_at | Cluster Incl AA674143 vp96d10 r1 Mus musculus cDNA, 5 end/clone = IMAGE-1092595/clone_end = 5/gb = AA674143/ | 503.8 | P | 474 | P | 414.5 | P | 469.9 |
| | | gi-2651380/ug = Mm.28894/len = 474/STRA = for | | | | | | | |
| 17 | 96736_at | Cluster Incl AI850370 UI-M-BG1-aig-c-02-0-UI s1 Mus musculus cDNA, 3 end/clone = UI-M-BG1-aig-c-02-0-UI/ | 941.5 | P | 924.2 | P | 828.6 | P | 637.3 |
| | | clone_end = 3/gb = AI850370/gi = 5494276/ug = Mm 28900/len = 432/STRA = rev | | | | | | | |
| 17 | 96750_at | Cluster Incl AW122179 UI-M-BH2 2-aov-a-01-0-UI s1 Mus musculus cDNA, 3 end/clone = UI-M-BH2 2-aov-a-01-0-UI/ | 416.4 | P | 356.1 | P | 370.9 | P | 243.3 |
| | | clone_end = 3/gb = AW122179/gi = 6097626/ug = Mm 28965/len = 496/STRA = for | | | | | | | |
| 17 | 96781_at | Cluster Incl AI838293.UI-M-AO0-aby-c-04-0-UI s1 Mus musculus cDNA, 3 end/clone = UI-M-AO0-aby-c-04-0-UI/ | 371.3 | P | 494.7 | P | 375.3 | P | 407.7 |
| | | clone_end = 3/gb = AI838293/gi = 5472506/ug = Mm 29072/len = 310/STRA = for | | | | | | | |
| 17 | 98020_at | Cluster Incl D10715.Mouse mRNA for novel GTP-binding protein, complete cds/cds = (76,1179)/gb = D10715/gi = 220506/ | 990.6 | P | 1039 | P | 902.4 | P | 953 |
| | | ug = Mm.3250/len = 1524/STRA = for | | | | | | | |
| 17 | 98032_at | Cluster Incl M36146 Zinc finger protein 35/cds = (164,1906)/gb = M36146/gi = 202451/ug = Mm.3297/len = 2293/STRA = for | 350.8 | P | 257.7 | P | 147.3 | P | 195.9 |
| 17 | 98397_at | Cluster Incl AI852694 UI-M-BH0-aji-d-08-0-UI.s1 Mus musculus cDNA, 3 end/clone = UI-M-BH0-aji-d-08-0-UI/clone_end = 3/ | 269.8 | P | 302.2 | P | 228.8 | P | 223.5 |
| | | gb = AI852694/gi = 5496600/ug = Mm 33285/len = 495/STRA = for | | | | | | | |
| 17 | 98464_at | Cluster Incl AW124196 UI-M-BH2 1-aph-b-03-0-UI s1 Mus musculus cDNA, 3 end/clone = UI-M-BH2 1-aph-b-03-0-UI/ | 301.1 | P | 288.7 | P | 266.5 | P | 245.6 |
| | | clone_end = 3/gb = AW124196/gi = 6099726/ug = Mm 34812/len 382/STRA = rev | | | | | | | |
| 17 | 98486_at | Cluster Incl AF105222 Mus musculus slathmin-like-protein RB3 mRNA, complete cds/cds = (88,657)/gb = AF105222/ | 513.2 | P | 315.7 | P | 667.3 | P | 482.7 |
| | | gi = 4028597/ug = Mm 35474/len = 1197/STRA = rev | | | | | | | |
| 17 | 98982_at | Cluster Incl AW046443 UI-M-BH1-akp-e-04-0-UI s1 Mus musculus cDNA, 3 end/clone = UI-M-BH1-akp-e-04-0-UI/ | 951.2 | P | 753.1 | P | 723.9 | P | 709.5 |
| | | clone_end = 3/gb = AW046443/gi = 5906972/ug = Mm.36912/len = 439/STRA = for | | | | | | | |
| 17 | 99485_at | Cluster Incl AB009376 Mus musculus mRNA for ICAD-S, complete cds/cds = (0,797)/gb = AB009376/gi = 2754587/ | 331.1 | P | 295.1 | P | 209.6 | P | 286.5 |
| | | ug = Mm 41433/len = 798/STRA = for | | | | | | | |
| 17 | 99536_at | Cluster Incl AB016080 Mus musculus mRNA for calcium binding protein Ktp 2, complete cds/cds = (61,624)/gb = AB016080/ | 211 | P | 162.7 | P | 144.7 | P | 181.7 |
| | | gi = 4115809/ug = Mm.42192/len = 736/STRA = for | | | | | | | |
| 17 | 99549_at | Cluster Incl D31951: Mouse mRNA for osteoglycin, complete cds/cds = (39,935)/gb = D31951/gi = 971273/ug = Mm.4258/ | 223.2 | P | 81.1 | P | 92.4 | P | 61.3 |
| | | len = 1268/STRA = for | | | | | | | |
| 17 | 99975_at | Cluster Incl AA879937: vw02b10.r1 Mus musculus cDNA, 5 end/clone = IMAGE-1230715/clone_end = 5/gb = AA879937/ | 432.8 | P | 388.9 | P | 288 | P | 325.7 |
| | | gi = 2988920/ug = Mm 4428/len = 331/STRA = for | | | | | | | |
| 17 | 100009_r_at | Cluster Incl X94127 SRY-box containing gene 2/cds = (0,959)/gb = X94127/gi = 1209429/ug = Mm 4541/len = 960/STRA = for | 105.2 | P | 115 | P | 70.8 | P | 64.9 |
| 17 | 100496_at | Cluster Incl U79523 Mus musculus peptidylglycine alpha-amidating monooxygenase (PAM) mRNA, complete cds/ | 339.1 | P | 632.2 | P | 315.3 | P | 395 |
| | | cds = (335,3274)/gb = U79523/gi = 1711198/ug = Mm.5121/len = 4032/STRA = for | | | | | | | |
| 17 | 101877_at | Cluster Incl AI854432 UI-M-BH0-ail-c-06-0-UI s1 Mus musculus cDNA, 3 end/clone = UI-M-BH0-ail-c-06-0-UI/clone_end = 3/ | 562.2 | P | 456 | P | 469.4 | P | 442.9 |
| | | gb = AI854432/gi = 5498338/ug = Mm 88182/len = 413/STRA = rev | | | | | | | |
| 17 | 101912_at | Cluster Incl AI019679 ua92e01.r1 Mus musculus cDNA, 5 end/clone = IMAGE-1364952/clone_end = 5/gb = AI019679/ | 2564.5 | P | 2384 | P | 1224.9 | P | 2320.7 |
| | | gi = 3234015/ug = Mm 902/len = 363/STRA = for | | | | | | | |
| 17 | 102215_at | Cluster Incl Y10495 Mus musculus mRNA for CDV-1R protein/cds = (886,2130)/gb = Y10495/gi = 2995446/ug = Mm 10125/ | 256 | P | 204.7 | P | 154.4 | P | 180.6 |
| | | len = 2649/STRA = for | | | | | | | |
| 17 | 102894_g_at | Cluster Incl X68363 POU domain, class 2, transcription factor 1/cds = (52,2292)/gb = X68363/gi = 53471/ug = Mm 15568/ | 347.1 | P | 188.9 | P | 183.1 | P | 244.5 |
| | | len = 2574/STRA = for | | | | | | | |
| 17 | 102980_at | Cluster Incl AI849119 UI-M-AJ1-agy-a-03-0-UI s1 Mus musculus cDNA, 3 end/clone = UI-M-AJ1-agy-a-03-0-UI/ | 459 | P | 311.3 | P | 467.4 | P | 433.2 |
| | | clone_end = 3/gb = Al849119/gi = 5493025/ug = Mm.1896/len = 507/STRA = rev | | | | | | | |
| 17 | 103285_at | Cluster Incl AF072249.Mus musculus methyl-CpG binding protein MBD4 (Mbd4) mRNA, complete cds/cds = (148,1812)/ | 108.8 | P | 125.5 | P | 46.5 | P | 222.7 |
| | | gb = AF072249/gi = 3800806/ug = Mm 20890/len = 1955/STRA = for | | | | | | | |
| 17 | 103713_at | Cluster Incl AW124533 UI-M-BH2 1-apo-g-12-0-UI s1 Mus musculus cDNA, 3 end/clone = UI-M-BH2 1-apo-g-12-0-UI/ | 698.2 | P | 649.5 | P | 474.6 | P | 631.5 |
| | | clone_end = 3/gb = AW124533/gi = 6100063/ug = Mm 24116/len = 379/STRA = for | | | | | | | |
| 17 | 103780_at | Cluster Incl AW049510 UI-M-BH1-anf-b-05-0-UI s1 Mus musculus cDNA, 3 end/clone = UI-M-BH1-anf-b-05-0-UI/ | 228.7 | P | 254.2 | P | 210.4 | P | 190.5 |
| | | clone_end = 3/gb = AW049510/gi = 5910039/ug = Mm 24863/len = 458/STRA = for | | | | | | | |
| 17 | 103781_at | Cluster Incl U76832 Mus musculus plasma membrane protein syntaxin-4 mRNA, complete cds/cds = (0,896)/gb = U76832/ | 648.2 | P | 605 | P | 447.8 | P | 488.9 |
| | | gi = 1673624/ug = Mm 24867/len = 897/STRA = for | | | | | | | |
| 17 | 103916_at | Cluster Incl AI850713 UI-M-BG1-ail-d-10-0-UI s1 Mus musculus cDNA, 3 end/clone = UI-M-BG1-ail-d-10-0-UI/ | 363.1 | P | 295.9 | P | 251.5 | P | 310.8 |
| | | clone_end = 3/gb = AI850713/gi = 5494619/ug = Mm 24984/len = 321/STRA = rev | | | | | | | |
| 17 | 103981_at | Cluster Incl AI842281 UI-M-AM1-afz-e-07-0-UI s1 Mus musculus cDNA, 3 end/clone = UI-M-AM1-afz-e-07-0-UI/ | 278.7 | P | 241 | P | 243.6 | P | 146.4 |
| | | clone_end = 3/gb = AI842281/gi = 5476534/ug = Mm 25837/len = 457/STRA = for | | | | | | | |
| 17 | 104652_at | Cluster Incl AI849601 UI-M-AH1-agr-f-05-0-UI s1 Mus musculus cDNA, 3 end/clone = UI-M-AH1-agr-f-05-0-UI/ | 211.6 | P | 164.1 | P | 163.9 | P | 141.4 |
| | | clone_end = 3/gb = AI849601/gi = 5493507/ug = Mm 31570/len = 357/STRA = rev | | | | | | | |
| 17 | 92241_at | Cluster Incl AI849075.UI-M-AH1-agw-e-03-0-UI s1 Mus musculus cDNA, 3 end/clone = UI-M-AH1-agw-e-03-0-UI/ | 603.3 | P | 678.6 | P | 491.1 | P | 330.5 |
| | | clone_end = 3/gb = AI849075/gi = 5492981/ug = Mm 34844/len = 355/STRA = rev | | | | | | | |
| 17 | 92350_at | Cluster Incl AA165759 ms60e10 r1 Mus musculus cDNA, 5 end/clone = IMAGE-615978/clone_end = 5/gb = AA165759/ | 585.8 | P | 668.4 | P | 518.3 | P | 525.8 |
| | | gi = 1743974/ug = Mm 3926/len = 346/STRA = rev | | | | | | | |
| 17 | 92495_at | Cluster Incl U71269 Mus musculus potential transcriptional repressor No14hp (Not4h) mRNA, complete cds/ | 275.7 | P | 236.3 | P | 218.4 | P | 171.2 |
| | | cds = (314,2041)/gb = U71269/gi = 4097901/ug = Mm 42183/len = 2319/STRA = rev | | | | | | | |
| 17 | 92698_at | Cluster Incl U21301 Mus musculus c-mer tyrosine kinase receptor mRNA, complete cds/cds = (49,3033)/gb = U21301/ | 134.5 | P | 146.4 | P | 149.4 | P | 128.1 |
| | | gi = 885969/ug = Mm 4582/len = 3564/STRA = for | | | | | | | |
| 17 | 92904_at | Cluster Incl U08185 B lymphocyte induced maturation protein/cds = (220,2790)/gb = U08185/gi = 474283/ug = Mm 4800/ | 213.4 | P | 215.7 | P | 195.7 | P | 190.8 |
| | | len = 5281/STRA = rev | | | | | | | |
| 17 | 92986_g_at | Cluster Incl D83203 Mus musculus mRNA, one isoform of PTP-RL9/cds = UNKN0WN/gb = D83203/gi = 4092839/ | 491.3 | P | 436.3 | P | 287.2 | P | 365.5 |
| | | ug = Mm 5046/len = 4208/STRA = for | | | | | | | |
| 17 | 93169_at | Cluster Incl AW212708 uo10g07.x1 Mus musculus cDNA, 3 end/clone = IMAGE-2599164/clone_end = 3/gb = AW212708/ | 361.1 | P | 446.6 | P | 185.5 | P | 373.2 |
| | | gi = 6518827/ug = Mm 5343/len = 465/STRA = for | | | | | | | |
| 17 | 93456_r_at | Cluster Incl L47480.Mus musculus BMP-4 gene, complele cds/cds = (92,1354)/gb = L47480/gi = 994733/ug = Mm 6813/ | 309.4 | P | 403.7 | P | 270.5 | P | 318 |
| | | len = 1654/STRA = for | | | | | | | |
| 17 | 93623_at | Cluster Incl U56724 Mus musculus dystrophin gene, exon 23 and partial cds/cds = (0,212)/gb = U56724/gi = 1336044/ | 210.3 | P | 176.6 | P | 112.2 | P | 105.3 |
| | | ug = Mm 742/len = 213/STRA = for | | | | | | | |
| 17 | 93962_at | Cluster Incl AI848598 UI-M-AH1-agv-b-04-0-UI s1 Mus musculus cDNA, 3 end/clone = UI-M-AH1-agv-b-04-0-UI/ | 264.3 | P | 201.1 | P | 173.6 | P | 177.3 |
| | | clone_end = 3/gb = AI848598/gi = 5492544/ug = Mm 967/len = 470/STRA = for | | | | | | | |
| 17 | 95287_at | Cluster Incl AA189677 mt70h07 rl Mus musculus cDNA, 5 end/clone = IMAGE-635293/clone_end = 5/gb = AA189677/ | 421.5 | P | 421.5 | P | 316.2 | P | 362.4 |
| | | gi = 1776319/ug = Mm 1873/len = 305/STRA = for | | | | | | | |
| 17 | 95343_at | Cluster Incl AB013848 Mus musculus mRNA for peptidylarginine deiminase type I, complete cds/cds = (127,2115)/ | 248.1 | P | 230 | P | 152.9 | P | 183.1 |
| | | gb = AB013848/gi = 3845556/ug = Mm 20854/len = 3755/STRA = for | | | | | | | |
| 17 | 95356_at | Cluster Incl D00466 Apolipoprotein E/cds = (23,958)/gb = D00466/gi = 220334/ug = Mm.21417/len = 1068/STRA = for | 3625.6 | P | 3395 | P | 3337.9 | P | 3520.2 |
| 17 | 95368_at | Cluster Incl D86949.Mouse mRNA for plexin 2, complete cds/cds = (579,6233)/gb = D86949/gi = 1655431/ug = Mm 2251/ | 437.1 | P | 335.6 | P | 281.4 | P | 225.8 |
| | | len = 6730/STRA = for | | | | | | | |
| 17 | 96564_at | Cluster Incl X54401 Mouse U14 snRNA (intron 5 of hsc70 gene)/cds = (0,155)/gb = X54401/gi = 55071/ug = Mm 2944/ | 4111.2 | P | 4135.2 | P | 3271.5 | P | 3925.1 |
| | | len = 156/STRA = for | | | | | | | |
| 17 | 102124_f_at | Cluster Incl AI836879 UI-M-AP0-abg-d-08-0-UI.s2 Mus musculus cDNA, 3 end/clone = UI-M-AP0-abg-d-08-0-UI/ | 524 | P | 1061.8 | P | 775.5 | P | 904.7 |
| | | clone_end = 3/gb = AI836879/gi = 5471092/ug = Mm 43075/len = 252/STRA = rev | | | | | | | |
| 17 | 102141_f_at | Cluster Incl AI462340 ub72a07 x1 Mus musculus cDNA, 3 end/clone = IMAGE-1383252/clone_end = 3/gb = AI462340/ | 428.5 | P | 441.7 | P | 257.9 | P | 321.3 |
| | | gi = 4316370/ug = Mm 44054/len = 339/STRA = rev | | | | | | | |
| 17 | 102171_r_at | Cluster Incl AI132241 ue32f10 x1 Mus musculus cDNA, 3 end/clone = IMAGE-1482091/clone_end = 3/gb = AI132241/ | 1077.3 | P | 863.9 | P | 935.6 | P | 710.3 |
| | | gi = 3602257/ug = Mm 46845/len = 735/STRA = rev | | | | | | | |
| 17 | AFFX-BioB- | E Coli BioB 5 prime | 518.2 | P | 577.2 | P | 352.7 | P | 486.9 |
| | 5_at | | | | | | | | |
| 17 | AFFX-b- | M12481 Mouse cytoplasmic beta-actin mRNA (_5, _M, _3 represent transcript regions 5 prime, | 133.5 | P | 97.9 | P | 146.4 | P | 119.9 |
| | ActinMur/ | Middle, and 3 prime respectively) | | | | | | | |
| | M12481_{—} | | | | | | | | |
| | M_at | | | | | | | | |
| 17 | 93040_at | Cluster Incl AF091390 Mus musculus phospholemman precursor, gene, complete cds/cds = (0,278)/gb = AF091390/ | 980 | P | 752 | P | 870.5 | P | 746.4 |
| | | gi = 4206710/ug = Mm.1491/len = 279/STRA = for | | | | | | | |
| 17 | 94214_at | Cluster Incl X14961: Fatty acid binding protein heart 1/cds = (21,422)/gb = X14961/gi = 51266/ug = Mm.22220/len = 658/ | 203.6 | P | 171.2 | P | 101 | A | 213.8 |
| | | STRA = for | | | | | | | |
| 17 | 96904_at | Cluster Incl AI848784: UI-M-AJ1-ahg-c-10-0-Ul.s1 Mus musculus cDNA, 3 end/clone = UI-M-AJ1-ahg-c-10-0-UI/ | 274.8 | P | 329.2 | P | 346.3 | P | 334.1 |
| | | clone_end = 3/gb = AI848784/gi = 5492690/ug = Mm 30123/len = 490/STRA = for | | | | | | | |
| 17 | 98061_at | Cluster Incl AI841192: UI-M-AM0-ad1-e-02-0-UI s1 Mus musculus cDNA, 3 end/clone = UI-M-AM0-adl-e-02-0-UI/ | 442.7 | P | 564.9 | P | 300.4 | P | 284.2 |
| | | clone_end = 3/gb = AI841192/gi = 5475405/ug = Mm 34386/len = 444/STRA = for | | | | | | | |
| 17 | 99658_f_at | Cluster Incl AW047907: UI-M-BH1-aln-b-05-0-UI s1 Mus musculus cDNA, 3 end/clone = UI-M-BH1-aln-b-05-0-UI/ | 685 | P | 633 | P | 401.1 | A | 539.8 |
| | | clone_end = 3/gb = AW047907/gi = 5908436/ug = Mm 43784/len = 398/STRA = for | | | | | | | |
| 17 | 101546_at | Cluster Incl AF109906 Mus musculus MHC class III region RD gene, partial cds, Bf, C2, G9A, NG22, G9, HSP70, HSP70, | 244.2 | P | 252.6 | P | 198.8 | A | 241 |
| | | HSC70t, and smRNP genes, complete cds, G7A gene, partial cds, and unknown genes/cds = (0,1229)/gb = AF109906/ | | | | | | | |
| | | gi = 3986763/ug = Mm 8856/len = 1230/STRA = for | | | | | | | |
| 17 | 103913_at | Cluster Incl AI851315 UI-M-BH0-akf-h-11-0-UI s1 Mus musculus cDNA, 3 end/clone = UI-M-BH0-akf-h-11-0-UI/ | 246.8 | P | 274.4 | P | 240.5 | P | 195.8 |
| | | clone_end = 3/gb = AI851315/gi = 5495221/ug = Mm.21435/len = 336/STRA = for | | | | | | | |
| 17 | 104349_at | Cluster Incl AA870301: vq14c07 r1 Mus musculus cDNA, 5 end/clone = IMAGE-1094220/clone_end = 5/gb = AA870301/ | 222.3 | P | 269.6 | P | 117.8 | A | 142 |
| | | gi = 2965746/ug = Mm.22548/len = 429/STRA = for | | | | | | | |
| 17 | 104356_at | Cluster Incl AI465543 mu92f12.x1 Mus musculus cDNA, 3 end/clone = IMAGE-653039/clone_end = 3/gb = AI465543/ | 124 | P | 149.6 | P | 139.8 | P | 99.6 |
| | | gi = 4319573/ug = Mm.22581/len = 184/STRA = for | | | | | | | |
| 17 | 94365_at | Cluster Incl AA710175 vt49a11 r1 Mus musculus cDNA, 5 end/clone = IMAGE-1166396/clone_end = 5/gb = AA710175/ | 555.4 | P | 600.1 | A | 476.7 | P | 469.8 |
| | | gi = 2720093/ug = Mm.25285/len = 481/STRA = for | | | | | | | |
| 17 | 95633_r_at | Cluster Incl AW122653 UI-M-BH2.2-aol-e-11-0-UI s1 Mus musculus cDNA, 3 end/clone = UI-M-BH2 2-aol-e-11-0-UI/ | 540.4 | P | 554.7 | P | 399.6 | P | 474.7 |
| | | clone_end = 3/gb = AW122653/gi = 6098183/ug = Mm 28088/len = 363/STRA = for | | | | | | | |
| 17 | 96732_at | Cluster Incl AI851081 UI-M-BH0-akg-a-12-0-UI s1 Mus musculus cDNA, 3 end/clone = UI-M-BH0-akg-a-12-0-UI/ | 440.6 | P | 580.9 | A | 373 | P | 425.9 |
| | | clone_end = 3/gb = AI851081/gi = 5494987/ug = Mm 28891/len = 480/STRA = for | | | | | | | |
| 17 | 99455_s_at | Cluster Incl D17574 Mouse mRNA for proacrosin-binding protein (sp32), complete cds (alternative splicing product)/ | 292.1 | P | 321.8 | P | 268.9 | P | 265.7 |
| | | cds = (87,1037)/gb = D17574/gi = 498163/ug = Mm 4073/len = 1253/STRA = for | | | | | | | |
| 17 | 99505_at | Cluster Incl AI844664 UI-M-AL1-ahr-d-11-0-UI.s1 Mus musculus cDNA, 3 end/clone = UI-M-AL1-ahr-d-11-0-UI/ | 279.5 | P | 310.8 | P | 373 | P | 295.4 |
| | | clone_end = 3/gb = AI844664/gi = 5488570/ug = Mm 41750/len = 434/STRA = for | | | | | | | |
| 17 | 99527_at | Cluster Incl AB013852 Mus musculus mRNA for Nrf3, complete cds/cds = (237,2219)/gb = AB013852/gi = 4521228/ | 138.3 | P | 67.7 | P | 62.8 | A | 86.8 |
| | | ug = Mm 42138/len = 2544/STRA = for | | | | | | | |
| 17 | 100982_at | Cluster Incl AI854539 UI-M-BH0-ajj-e-10-0-UI s1 Mus musculus cDNA, 3 end/clone = UI-M-BHO-ajj-e-10-0-UI/clone_end = 3/ | 157.5 | P | 156.8 | P | 136.9 | P | 77.4 |
| | | gb = AI854539/gi= 5498445/ug = Mm 6720/len = 461/STRA = for | | | | | | | |
| 17 | 101397_at | Cluster Incl AA870756 vq26b08 r1 Mus musculus cDNA, 5 end/clone = IMAGE-1095351/clone_end = 5/gb = AA870756/ | 536.7 | P | 400.8 | P | 544.7 | P | 465.2 |
| | | gi = 2966201/ug = Mm 7241/len = 281/STRA = for | | | | | | | |
| 17 | 101919_at | Cluster Incl M32309.Zinc finger protein X-linked/cds = (198,2717)/gb = M32309/gi = 202455/ug = Mm 919/len = 6878/ | 285.8 | P | 297.2 | P | 125.6 | P | 251 |
| | | STRA = for | | | | | | | |
| 17 | 102193_at | Cluster Incl AA791891 vs54hO4.r1 Mus musculus cDNA, 5 end/clone = IMAGE-1150135/clone_end = 5/gb = AA791891/ | 358.3 | P | 259.6 | P | 103.4 | A | 183.6 |
| | | gi = 2854846/ug = Mm 9821/len = 418/STRA = for | | | | | | | |
| 17 | 102196_at | Cluster Incl U37413 Guanine nucleotide binding protein, alpha 11/cds = (57,1136)/gb = U37413/gi = 1051237/ug = Mm 989/ | 226.5 | P | 378.8 | P | 198.9 | P | 253.6 |
| | | len = 1342/STRA = rev | | | | | | | |
| 17 | 103315_at | Cluster Incl AW120526 UI-M-BH1-ana-b-09-0-UI s2 Mus musculus cDNA, 3 end/clone = UI-M-BH1-ana-b-09-0-UI/ | 175.7 | P | 258.4 | P | 161.9 | P | 119.5 |
| | | clone_end = 3/gb = AW120526/gi = 6095859/ug = Mm 21289/len = 426/STRA = for | | | | | | | |
| 17 | 104007_at | Cluster Incl AA986782 uc74c02 x1 Mus musculus cDNA, 3 end/clone = IMAGE-1431362/clone_end = 3/gb = AA986782/ | 319.2 | P | 233.6 | P | 216.9 | P | 286.6 |
| | | gi = 3167784/ug = Mm 26737/len = 394/STRA = rev | | | | | | | |
| 17 | 104398_at | Cluster Incl AI846222 UI-M-AM1-agb-g-06-0-UI s1 Mus musculus cDNA, 3 end/clone = UI-M-AM1-agb-g-06-0-UI/ | 146.1 | P | 149.9 | A | 79.2 | P | 171.7 |
| | | clone_end = 3/gb = AI846222/gi = 5490128/ug = Mm 28655/len = 232/STRA = for | | | | | | | |
| 17 | 92330_r_at | Cluster Incl AA517665 vh81f01 r1 Mus musculus cDNA, 3 end/clone = IMAGE-893401/clone_end = 3/gb = AA517665/ | 235.8 | P | 203.6 | P | 179.9 | P | 180.8 |
| | | gi = 2257189/ug = Mm 38891/len = 484/STRA = rev | | | | | | | |
| 17 | 92711_at | Cluster Incl M57625 Mast cell protease 6/cds = UNKNOWN/gb = M57625/gi = 200506/ug = Mm 46216/len = 3757/STRA = for | 244.6 | P | 191.2 | P | 172.8 | P | 190.9 |
| 17 | 93130_at | Cluster Incl AW122860 UI-M-BH2.1-aoz-h-09-0-UI s1 Mus musculus cDNA, 3 end/clone = UI-M-BH2 1-aoz-h-09-0-UI/ | 297.4 | P | 217 | P | 178.7 | P | 185 |
| | | clone_end = 3/gb = AW122860/gi = 6098390/ug = Mm 5117/len = 186/STRA = for | | | | | | | |
| 17 | 97706_at | Cluster Incl AA474655 vd53b01.s1 Mus musculus cDNA, 5 end/clone = IMAGE-804265/clone_end = 5/gb = AA474655/ | 119.2 | P | 143.7 | P | 145.6 | P | 91.5 |
| | | gi = 2202810/ug = Mm 38051/len = 305/STRA = for | | | | | | | |
| 17 | 99856_r_at | Cluster Incl AI852919 UI-M-BH0-aiz-b-06-0-UI.s1 Mus musculus cDNA, 3 end/clone = UI-M-BH0-aiz-b-06-0-UI/ | 220.9 | P | 227.5 | P | 187.3 | P | 177.5 |
| | | clone_end = 3/gb = AI852919/gi = 5496825/ug = Mm 6680/len = 396/STRA = rev | | | | | | | |
| 17 | 101759_at | Cluster Incl D00851: Mus musculus Tcp-1 pseudogene for I-complex polypeptide 1/cds = (0,1352)/gb = D00851/gi = 220608/ | 55.6 | M | 89.2 | P | 64.2 | P | 76 6 |
| | | ug = Mm 57089/len = 1353/STRA = for | | | | | | | |
| 17 | 102583_at | Cluster Incl AJ132195 Mus musculus olfr89 gene, partial/cds = (0,908)/gb = AJ132195/gi = 4186074/ug = Mm 57248/len = 909/ | 210.5 | P | 298.2 | P | 214.1 | P | 189.3 |
| | | STRA = for | | | | | | | |
| 17 | 100751_at | Cluster Incl AF011379 A disintegrin and metalloprotease domain (ADAM) 10/cds = (25,2274)/gb = AF011379/gi = 2282607/ | 255.2 | P | 284.8 | P | 113.4 | P | 206.7 |
| | | ug = Mm 3911/len = 2481/STRA = for | | | | | | | |
| 17 | 93311_at | Cluster Incl AF033565: Mus musculus cdc2/CDC28-like protein kinase 3 (Clk3) mRNA, complete cds/cds = (63,1535)/ | 43.6 | P | −52.1 | A | 124.6 | P | 11.4 |
| | | gb = AF033565/gi = 2645653/ug = Mm 18476/len = 1787/STRA = for | | | | | | | |
| 17 | 97870_s_at | Cluster Incl AA798624 vy03d11.r1 Mus musculus cDNA, 5 end/clone = IMAGE-1294389/clone_end = 5/gb = AA798624/ | 420 | P | 234.2 | P | 150.1 | P | 139 |
| | | gi = 2861579/ug = Mm 28339/len = 280/STRA = rev | | | | | | | |
| 17 | 98109_at | Cluster Incl AI839526 UI-M-ANO-acn-e-06-0-UI s1 Mus musculus cDNA, 3 end/clone = UI-M-AN0-acn-e-06-0-UI/ | 197.3 | A | 201.2 | P | 160 | P | 153.4 |
| | | clone_end = 3/gb = AI839528/gi = 5473741/ug = Mm 34801/len = 355/STRA = for | | | | | | | |
| 17 | 99144_s_at | Cluster Incl D50031: Mouse mRNA for TGN38A, complete cds/cds = (26,1087)/gb = D50031/gi = 949828/ug = Mm 4142/ | 282.7 | P | 209.6 | A | 216.9 | P | 152.7 |
| | | len = 1673/STRA = for | | | | | | | |
| 17 | 103028_at | Cluster Incl D14042 Mus musculus tlk mRNA for tyrosine kinase, complete cds/cds = (45,1904)/gb = D14042/gi = 220613/ | 359 | P | 274.2 | P | 130.1 | P | 180.3 |
| | | ug = Mm 16009/len = 4224/STRA = for | | | | | | | |
| 17 | 103057_at | Cluster Incl AF024570: DNA polymerase delta 1, calalytic domain/cds = (0,3317)/gb = AF024570/gi = 2827902/ | 468.4 | P | 417.9 | P | 120.2 | P | 347.1 |
| | | ug = Mm.16549/len = 3318/STRA = rev | | | | | | | |
| 17 | 103914_at | Cluster Incl AI847114: UI-M-AP1-agn-h-04-0-UI s1 Mus musculus cDNA, 3 end/clone = UI-M-AP1-agn-h-04-0-UI/ | 644.7 | P | 701.7 | P | 515.2 | A | 568 |
| | | clone_end = 3/gb = AI847114/gi = 5491025/ug = Mm.21439/len = 416/STRA = for | | | | | | | |
| 17 | 95604_at | Cluster Incl AI843294: UI-M-AO1-ael-f-03-0-UI s1 Mus musculus cDNA, 3 end/clone = UI-M-AO1-aei-f-03-0-UI/ | 473.7 | P | 699.5 | P | 510.2 | M | 623.6 |
| | | clone_end = 3/gb = A1843294/gi = 5477507/ug = Mm 27954/len = 376/STRA = for | | | | | | | |
| 17 | 102698_at | Cluster Incl AF045160 Mus musculus hypoxia-inducible factor 1 alpha related factor mRNA, complete cds/cds = (183,2807)/ | 228.3 | A | 188.1 | P | 260.4 | P | 223.5 |
| | | gb = AF045160/gi = 2895755/ug = Mm.1415/len = 3415/STRA = for | | | | | | | |
| 17 | 103438_at | Cluster Incl AF096875 Mus musculus type 2 deiodinase mRNA, complete cds/cds = (154,954)/gb = AF096875/gi = 4206102/ | 140.3 | P | 101.7 | P | 96.4 | P | 85.9 |
| | | ug = Mm 21389/len = 5841/STRA = for | | | | | | | |
| 17 | 104480_at | Cluster Incl AI152659 ud93d03 r1 Mus musculus cDNA, 5 end/clone = IMAGE-1478405/clone_end = 5/gb = AI152659/ | 341.8 | P | 279.1 | P | 270.3 | P | 356.6 |
| | | gi = 3681128/ug = Mm.30027/len = 334/STRA = for | | | | | | | |
| 17 | 92287_at | Cluster Incl AF033187 Mus musculus SOCS-5 mRNA, complete cds/cds = (0,1610)/gb = AF033187/gi = 2766490/ | 309.1 | P | 324.8 | P | 250.6 | M | 339.9 |
| | | ug = Mm.37204/len = 1611/STRA = for | | | | | | | |
| 17 | 92710_at | Cluster Incl U73199 Mus musculus Rho-guanine nucleotide exchange factor mRNA, complete cds/cds = (104,5185)/ | 168.5 | P | 71.7 | P | 115.1 | P | 135.8 |
| | | gb = U73199/gi = 1657834/ug = Mm 4620/len = 5405/STRA = for | | | | | | | |
| 17 | 93427_at | Cluster Incl AW122310 UI-M-BH2 2-aos-d-10-0-UI s1 Mus musculus cDNA, 3 end/clone = UI-M-BH2.2-aos-d-10-0-UI/ | 297.9 | M | 167.5 | P | 233.2 | P | 179.5 |
| | | clone_end = 3/gb = AW122310/gi = 6097795/ug = Mm.6493/len = 208/STRA = rev | | | | | | | |
| 17 | 94695_at | Cluster Incl C81411 C81411 Mus musculus cDNA, 3 end/clone = J0098H06/clone_end = 3/gb = C81411/gi = 2521741/ | 317.9 | P | 205 | P | 89.1 | A | 105.8 |
| | | ug = Mm 13352/len = 586/STRA = rev | | | | | | | |
| 17 | 102584_at | Cluster Incl Y18260 Mus musculus mRNA for protein kinase Dyrk1B/cds = (248,2017)/gb = Y18280/gi = 5738713/ | 295.6 | M | 322.6 | P | 210.7 | P | 198.3 |
| | | ug = Mm 57249/len = 2018/STRA = for | | | | | | | |
| 17 | 95782_at | Cluster Incl U19033 Melanoma antigen, related sequence 2/cds = (0,992)/gb = U19033/gi = 1165173/ug = Mm 87790/ | 181.5 | P | 217.5 | P | 135.9 | A | 129.5 |
| | | len = 993/STRA = for | | | | | | | |
| 17 | 100713_at | Cluster Incl AB020542: Mus musculus mRNA for Zinc finger protein sf 1-6, complete cds/cds = (106,1869)/gb = AB020542/ | 183.5 | P | 109.6 | P | 71 | A | 141.2 |
| | | gi = 3953592/ug = Mm.26510/len = 2363/STRA = for | | | | | | | |
| 17 | 92824_at | Cluster Incl AF051942 Mus musculus type 6 nucleoside diphosphate kinase NM23-M6 (Nm23-M6) mRNA, complete cds/ | 395 | P | 370 | P | 313.1 | P | 246.9 |
| | | cds = (121,690)/gb = AF051942/gi = 3228531/ug = Mm 12951/len = 1025/STRA = for | | | | | | | |
| 17 | 97490_at | Cluster Incl AJ011145 Mus musculus mRNA for protein encoded by BCL7B gene/cds = (78,623)/gb = AJ011145/ | 77.7 | P | 155.3 | P | 80.3 | P | 128.5 |
| | | gi = 3647321/ug = Mm 31/len = 1546/STRA = for | | | | | | | |
| 17 | 103801_at | Cluster Incl AF094761 Mus musculus Rfxank (Rfxank) mRNA, complete cds/cds = (201,1010)/gb = AF094761/gi = 3820617/ | 330.9 | P | 402.6 | P | 340.2 | P | 266.6 |
| | | ug = Mm 20848/len = 1403/STRA = for | | | | | | | |
| 17 | 97433_at | Cluster Incl AJ006590 Mus musculus mRNA for GANP protein/cds = (383,6298)/gb = AJ006590/gi = 4995702/ug = Mm 30098/ | 221.9 | P | 182.9 | P | 130.4 | P | 139.4 |
| | | len = 6429/STRA = for | | | | | | | |
| 17 | 103752_r_at | Cluster Incl AW227545 up10e09 x1 Mus musculus cDNA, 3 end/clone = IMAGE-2651656/clone_end = 3/gb = AW227545/ | 211.1 | A | 163.9 | P | 122.8 | A | 128.6 |
| | | gi = 6556841/ug = Mm 24516/len = 445/STRA = for | | | | | | | |
| 17 | 104246_at | Cluster Incl AI852340: UI-M-BH0-ajo-b-02-0-UI s1 Mus musculus cDNA, 3 end/clone = UI-M-BH0-ajo-b-02-0-UI/ | 338.6 | P | 432.8 | P | 284.6 | P | 312.3 |
| | | clone_end = 3/gb = AI852340/gi = 5496246/ug = Mm 2833/len = 501/STRA = for | | | | | | | |
| 17 | 104507_g_at | Cluster Incl AF009328 Mus musculus orphan nuclear hormone receptor isoform mCAR2 mRNA, complete cds/ | 269.9 | P | 268.6 | P | 245.1 | P | 200.6 |
| | | cds = (244,1104)/gb = AF009328/gi = 2267577/ug = Mm 3077/len = 1332/STRA = for | | | | | | | |
| 17 | 96531_at | Cluster Incl AA153773 mr77h06 r1 Mus musculus cDNA, 5 end/clone = IMAGE-603515/clone_end = 5/gb = AA153773/ | 255.8 | P | 275.9 | P | 240.5 | M | 285 |
| | | gi = 1724192/ug = Mm 27295/len = 455/STRA = for | | | | | | | |
| 17 | 96570_at | Cluster Incl AV381276: AV381276 Mus musculus cDNA, 3 end/clone = 9230111G20/clone_end = 3/gb = AV381276/ | 33.6 | P | 65.1 | P | 21.6 | P | 17.5 |
| | | gi = 6430353/ug = Mm 29740/len = 165/STRA = for | | | | | | | |
| 17 | 96600_at | Cluster Incl C76597 C76597 Mus musculus cDNA, 3 end/clone = J0014E03/clone_end = 3/gb = C76597/gi = 2516927/ | 82 | P | 71.1 | P | 47.4 | A | 66.2 |
| | | ug = Mm 31117/len = 601/STRA = rev | | | | | | | |
| 17 | 99364_at | Cluster Incl AI661370: va11c04 x1 Mus musculus cDNA, 3 end/clone = IMAGE-722598/clone_end = 3/gb = AI661370/ | 43.8 | A | 41.4 | P | 31 | A | 29.9 |
| | | gi = 4764953/ug = Mm 5633/len = 378/STRA = rev | | | | | | | |
| 17 | 100730_at | Cluster Incl D37797 Lymphocyte antigen 78/cds = (112,2097)/gb = D37797/gi = 761711/ug = Mm 3300/len = 2620/STRA = for | 132.2 | P | 135.9 | P | 92.9 | A | 108.9 |
| 17 | 92555_at | Cluster Incl AF053454 Mus musculus tetraspan TM4SF (Tspan-6) mRNA, complete cds/cds = (82,819)/gb = AF053454/ | 153.3 | P | 121.6 | P | 122.5 | P | 61.7 |
| | | gi = 2995862/ug = Mm 10180/len = 1058/STRA = for | | | | | | | |
| 17 | 98052_at | Cluster Incl AF003999 Mus musculus Golgi SNARE GS15 mRNA, complete cds/cds = (0,335)/gb = AF003999/gi = 2316079/ | 294.8 | A | 266.5 | P | 241 | P | 94.4 |
| | | ug = Mm.34315/len = 336/STRA = for | | | | | | | |
| 17 | 101501_r_at | Cluster Incl D87973 Imprinted and ancient/cds = (85,1041)/gb = D87973/gi = 4038075/ug = Mm 8154/len = 3442/STRA = for | 114.9 | P | 87.6 | A | 65.4 | A | 82.2 |
| 17 | 94363_at | Cluster Incl AI840815 UI-M-AH0-adb-c-03-0-UI s1 Mus musculus cDNA, 3 end/clone = UI-M-AH0-adb-c-03-0-UI/ | 192.2 | P | 154.6 | P | 160.2 | A | 118.5 |
| | | clone_end = 3/gb = AI840815/gi = 5475028/ug = Mm.25269/len = 363/STRA = for | | | | | | | |
| 17 | 94990_at | Cluster Incl AI854725 UI-M-BHO-akc-g-05-0-UI.s1 Mus musculus cDNA, 3 end/clone = UI-M-BH0-akc-g-05-0-UI/ | 130.6 | A | 227.9 | P | 117 | P | 143.8 |
| | | clone_end = 3/gb = AI854725/gi = 5498631/ug = Mm 27312/len = 512/STRA = for | | | | | | | |
| 17 | 96759_r_at | Cluster Incl AW061265: UI-M-BH1-amj-f-10-0-UI s2 Mus musculus cDNA, 3 end/clone = UI-M-BH1-amj-f-10-0-UI/ | 439.7 | P | 322 | P | 324.7 | P | 298.9 |
| | | clone_end = 3/gb = AW061265/gi = 6009016/ug = Mm 28994/len = 445/STRA = for | | | | | | | |
| 17 | 98965_at | Cluster Incl AC002397: Mouse chromosome 6 BAC-284H12 (Research Genetics mouse BAC library) complete sequence/ | 237 | A | 210.9 | A | 346.2 | P | 282.9 |
| | | cds = (1,1323)/gb = AC002397/gi = 3287367/ug = Mm 3634/len = 1741/STRA = rev | | | | | | | |
| 17 | 99537_at | Cluster Incl AF100694 Mus musculus Pontin52 mRNA, complete cds/cds = (144,15l4)/gb = AF100694/gi = 4106527/ | 246.9 | P | 263.9 | P | 383.1 | P | 148.7 |
| | | ug = Mm 42195/len = 1709/STRA = for | | | | | | | |
| 17 | 101873_at | Cluster Incl X95603: M musculus insl3 gene/cds = (3,368)/gb = X95603/gi = 1771291/ug = Mm 87963/len = 583/STRA = for | 118.4 | P | 57.6 | P | 64.5 | P | 85.5 |
| 17 | 103946_at | Cluster Incl U87814 Mus musculus PEST phosphatase Interacting protein mRNA, complete cds/cds = (273,1520)/ | 141 | P | 125.7 | P | 106.8 | A | 149 |
| | | gb = U87814/gi = 1857711/ug = Mm 2534/len = 1692/STRA = for | | | | | | | |
| 17 | 92246_at | Cluster Incl D85925 Mus musculus mRNA for anagen-specific protein mKAP13, complete cds/cds = (45,548)/gb = D85925/ | 206.1 | P | 144.5 | M | 150.6 | A | 180 |
| | | gi = 1944386/ug = Mm 3502/len = 844/STRA = for | | | | | | | |
| 17 | 93676_at | Cluster Incl U93583: RAD51 associated protein 1/cds = (43,1056)/gb = U93583/gi = 2697100/ug = Mm.8156/len = 1165/ | 406.3 | P | 352.5 | P | 268.6 | M | 180.2 |
| | | STRA = for | | | | | | | |
| 17 | 93933_at | Cluster Incl U89924: Mus musculus protein phosphatase 1 binding protein PTG mRNA, complete cds/cds = (60,944)/ | 184.4 | P | 330.6 | P | 211.7 | A | 186.7 |
| | | gb = U89924/gi = 1888565/ug = Mm 89946/len = 1169/STRA = for | | | | | | | |
| 17 | 95369_at | Cluster Incl AF021345 Mus musculus plasma selenoprotein P (SELP) gene, complete cds/cds = (56,1198)/gb = AF021345/ | 69.1 | P | 71.5 | P | 61.7 | A | 59.8 |
| | | gi = 4103141/ug = Mm 22699/len = 2823/STRA = for | | | | | | | |
| 17 | 95955_at | Cluster Incl AI606577 vb90e10 y1 Mus musculus cDNA, 5 end/clone = IMAGE-764298/clone_end = 5/gb = AI606577/ | 95.1 | P | 131.1 | P | 23.4 | P | 28.9 |
| | | gi = 4615744/ug = Mm.24932/len = 543/STRA = for | | | | | | | |
| 17 | 98367_at | Cluster Incl X73572 M musculus Hoxd-3 gene, complete CDS/cds = (218,865)/gb = X73572/gi = 313248/ug = Mm 4432/ | 59.5 | P | 47.8 | A | 64.7 | P | 28.4 |
| | | len = 2128/STRA = for | | | | | | | |
| 17 | 101157_at | Cluster Incl V00790 Part of messenger RNA for mouse delta-immunoglobulin (codes for part of exon 8 —one of two | 446.4 | P | 109 | A | 263.4 | P | 160.2 |
| | | alternate C-termini)/cds = (0,82)/gb = V00790/gi = 51778/ug = Mm.4939/len = 206/STRA = for | | | | | | | |
| 17 | 95670_at | Cluster Incl AI639868: UI-M-AH0-acw-a-05-0-UI s1 Mus musculus cDNA, 3 end/clone = UI-M-AH0-acw-a-05-0-UI/ | 177.4 | P | 201 | P | 144.8 | A | 161.6 |
| | | clone_end = 3/gb = AI839868/gi = 5474081/ug = Mm 29580/len = 406/STRA = for | | | | | | | |
| 17 | 101076_r_at | Cluster Incl AB016592 Mus musculus mRNA for GOB-4, complete cds/cds = (51,578)/gb = AB016592/gi = 3395165/ | 78.3 | P | 82 | M | 85.7 | P | 81.8 |
| | | ug = Mm.7244/len = 758/STRA = for | | | | | | | |
| 17 | 102352_at | Cluster Incl AW121380.UI-M-BH2 2-aom-f-06-0-UI s1 Mus musculus cDNA, 3 end/clone = UI-M-BH2 2-aom-f-06-0-UI/ | 114.5 | P | 91.4 | A | 92.1 | P | 15.7 |
| | | clone_end = 3/gb = AW121380/gi = 6096713/ug = Mm.11360/len = 526/STRA = rev | | | | | | | |
| 17 | 101908_s_at | Cluster Incl AF101164 Mus musculus biliary glycoprotein 2 long isoform (Bgp2) mRNA, complete cds/cds = υ,1120)/ | 20.8 | P | 36.2 | M | 29.8 | A | 41.9 |
| | | gb = AF101164/gi = 4092740/ug = Mm 89819/len = 1321/STRA = for | | | | | | | |
| 17 | 102217_at | Cluster Incl AI639925 vr74g08 x1 Mus musculus cDNA, 3 end/clone = IMAGE-1134494/clone_end = 3/gb = AI639925/ | 122.4 | P | 121 | P | 73.4 | A | 127.7 |
| | | gi = 4703034/ug = Mm 10188/len = 484/STRA = for | | | | | | | |
| 17 | 102240_at | Cluster Incl AF049330: Mus musculus PPAR gamma coactivator (PGC-1) mRNA, complete cds/cds = (91,2484)/ | 52.8 | A | 130.4 | P | 70.6 | A | 67.8 |
| | | gb = AF049330/gi = 3046930/ug = Mm 10707/len = 3029/STRA = for | | | | | | | |
| 17 | 103759_at | Cluster Incl D31788: BP-3 alloantigen/cds = (13,948)/gb = D31788/gi = 559368/ug = Mm.24612/len = 1255/STRA = for | 43 | P | 54.4 | P | 37.7 | A | 57.4 |
| 17 | 92507_at | Cluster Incl Y12229 Utrophin/cds = (0,10289)/gb = Y12229/gi = 1934962/ug = Mm.42222/len = 11096/STRA = for | 109.6 | P | 2.2 | A | 55.2 | P | 93.2 |
| 17 | 93923_at | Cluster Incl AW259620 up31b08 x1 Mus musculus cDNA, 3 end/clone = IMAGE-2655927/clone_end = 3/gb = AW259620/ | 369 | A | 567.5 | A | 300.3 | P | 310.6 |
| | | gi = 6632601/ug = Mm.89860/1en = 176/STRA = for | | | | | | | |
| 17 | 94124_at | Cluster Incl AB006074 Mus musculus mTOP3 mRNA for topoisomerase III, complete cds/cds = (265,3276)/gb = AB006074/ | 732.5 | A | 802.5 | P | 723 7 | P | 667.1 |
| | | gi = 3061307/ug = Mm 10708/len = 3741/STRA = for | | | | | | | |
| 17 | 95800_s_at | Cluster Incl X53250 Zinc finger protein, autosomal/cds = (0,2228)/gb = X53250/gi = 55468/ug = Mm 87837/len = 2229/ | 58.7 | P | 4.4 | A | 14 | A | 28.1 |
| | | STRA = for | | | | | | | |
| 17 | 96414_at | Cluster Incl D50656 Mus musculus mRNA for non-selective cation channel, complete cds/cds = (288,1560)/gb = D50656/ | 55.5 | P | 78 | A | 46.2 | M | 54.8 |
| | | gi = 2924621/ug = Mm.10098/len = 2785/STRA = for | | | | | | | |
| 17 | 102026_s_at | Cluster Incl AB011000 Mus musculus mRNA for choline/ethanolamine kinase, complete cds/cds = (299,1483)/ | 333.4 | P | 300.5 | M | 185.4 | M | 130.2 |
| | | gb = AB011000/gi = 2897728/ug = Mm.10124/len = 1708/STRA = for | | | | | | | |
| 17 | 102042_at | Cluster Incl AI842308: UI-M-AM1-afz-a-02-0-Ul.s1 Mus musculus cDNA, 3 end/clone = UI-M-AM1-afz-a-02-0-UI/ | 163.9 | A | 179.3 | P | 150.3 | P | 111.7 |
| | | clone_end = 3/gb = AI842308/gi = 5476486/ug = Mm 10200/len = 475/STRA = for | | | | | | | |
| 17 | 103663_at | Cluster Incl AI837330 UI-M-AK0-adf-d-04-0-UI s1 Mus musculus cDNA, 3 end/clone = UI-M-AK0-adt-d-04-0-UI/ | 169.1 | P | 142.2 | A | 73.3 | A | 119.5 |
| | | clone_end = 3/gb = AI837330/gi = 5471543/ug = Mm 2069/len = 332/STRA = for | | | | | | | |
| 17 | 95551_at | Cluster Incl AI849232 UI-M-AJ1-agz-c-07-0-UI.s1 Mus musculus cDNA, 3 end/clone = UI-M-AJ1-agz-c-07-0-UI/ | 218.3 | A | 293.3 | A | 239.4 | P | 220.8 |
| | | clone_end = 3/gb = AI849232/gi = 5493138/ug = Mm.27738/len = 419/STRA = for | | | | | | | |
| 17 | 104017_at | Cluster Incl AB033887 Mus musculus mACS4 vanant2 mRNA for Acyl-CoA synthetase 4 vanant2, complete cds/ | 50.1 | P | 16.9 | A | 44.9 | A | 22 |
| | | cds = (317,2452)/gb = AB033887/gi = 6172344/ug = Mm 26894/len = 2495/STRA = for | | | | | | | |
| 17 | 92344_at | Cluster Incl AF010600 Mus musculus TNF-response element binding protein mRNA, complete cds/cds = (177,2702)/ | 42.2 | P | 21 | P | 26.1 | A | 5.6 |
| | | gb = AF010600/gi = 2275596/ug = Mm.3909/len = 3064/STRA = for | | | | | | | |
| 17 | 92993_r_at | Cluster Incl AI324972: mm78d11 x1 Mus musculus cDNA, 3 end/clone = IMAGE-534549/clone_end = 3/gb = AI324972/ | 40.5 | A | 31.9 | M | 15.6 | A | 38.6 |
| | | gi = 4059401/ug = Mm 5073/len = 431/STRA = rev | | | | | | | |
| 17 | 96539_at | Cluster Incl AW212071 uo81h11.x1 Mus musculus cDNA, 3 end/clone = IMAGE-2648997/clone_end = 3/gb = AW212071/ | 94.7 | P | 21.7 | M | −27.3 | A | 1.1 |
| | | gi = 6518072/ug = Mm 27699/len = 402/STRA = for | | | | | | | |
| 17 | 97100_at | Cluster Incl AI854248 UI-M-BH0-ajz-h-11-0-UI.s1 Mus musculus cDNA, 3 end/clone = UI-M-BH0-ajz-h-11-0-UI/ | 90.7 | M | 92.5 | M | 71.3 | M | 75.3 |
| | | clone_end = 3/gb = AI854248/gi = 5498154/ug = Mm.32074/len = 516/STRA = rev | | | | | | | |
| 17 | 100739_at | Cluster Incl U96702.Mus musculus serine proteinase inhibitor mBM17 mRNA, partial cds/cds = (0,440)/gb = U96702/ | 41.9 | P | 52.6 | P | 51.4 | M | 65.5 |
| | | gi = 2104736/ug = Mm 3518/len = 594/STRA = for | | | | | | | |
| 18 | 92634_at | Cluster Incl U12620: Dipeptidylpeptidase 4/cds = (117,2399)/gb = U12620/gi = 1065904/ug = Mm.1151/len = 4943/STRA = for | 295.1 | P | 203.3 | P | 276.1 | P | 237.5 |
| 18 | 92643_at | Cluster Incl X74671 Neurofibromatosis 2/cds = (576,2363)/gb = X74671/gi = 456656/ug = Mm.1190/len = 2604/STRA = for | 375.9 | P | 301.6 | P | 344 | P | 311.4 |
| 18 | 92882_at | Cluster Incl Y00094 Mouse mRNA for ras-related YPT1 protein/cds = (129,746)/gb = Y00094/gi = 55456/ug = Mm 14530/ | 633.7 | P | 589.3 | P | 537.1 | P | 545.5 |
| | | len = 1428/STRA = for | | | | | | | |
| 18 | 93090_at | Cluster Incl M23362: Fibroblast growth factor receptor 2/cds = (0,1037)/gb = M23362/gi = 533219/ug = Mm.16340/len = 1038/ | 1183.1 | P | 1196.1 | P | 1173.8 | P | 1014 |
| | | STRA = for | | | | | | | |
| 18 | 93341_r_at | Cluster Incl AF043120 Mus musculus beta prime coatomer protein mRNA, partial cds/cds = (0,1352)/gb = AF043120/ | 883.4 | P | 1139.5 | P | 1060.6 | P | 1032.2 |
| | | gi = 2809536/ug = Mm.18729/1en = 1615/STRA = for | | | | | | | |
| 18 | 93509_at | Cluster Incl U57690: Ubiqultin-conjugating enzyme E2B (RAD6 homology)/cds = (43,501)/gb = U57690/gi = 1373352/ | 241.8 | P | 133 | P | 246.4 | P | 190.6 |
| | | ug = Mm 1920/1en = 923/STRA = for | | | | | | | |
| 18 | 93841_at | Cluster Incl AW060249 UI-M-BH1-amv-e-10-0-UI s1 Mus musculus cDNA, 3 end/clone = UI-M-BH1-amv-e-10-0-UI/ | 523.5 | P | 496.8 | P | 537.9 | P | 444.6 |
| | | clone_end = 3/gb = AW060249/gi = 6008040/ug = Mm 21617/len = 553/STRA = for | | | | | | | |
| 18 | 94223_at | Cluster Incl AJ010045: Mus musculus mRNA for Rho guanine nucleotide-exchange factor, splice variant NET1A/ | 1411.7 | P | 1522.3 | P | 1525.5 | P | 1200.7 |
| | | cds = (159,1784)/gb = AJ010045/gi = 4138206/ug = Mm 22261/len = 3290/STRA = for | | | | | | | |
| 18 | 94249_at | Cluster Incl AW226968: um64d03 y1 Mus musculus cDNA, 5 end/clone = IMAGE-2286149/clone_end = 5/gb = AW226968/ | 443.5 | P | 448.2 | P | 543.2 | P | 562.6 |
| | | gi = 6556264/ug = Mm 22374/len = 425/STRA = for | | | | | | | |
| 18 | 94882_at | Cluster Incl AA684506: vr02a10.r1 Mus musculus cDNA, 5 end/clone = IMAGE-1110618/clone_end = 5/gb = AA684506/ | 328.9 | P | 214.8 | P | 289 | P | 378.5 |
| | | gi = 2670223/ug = Mm.23877/len = 487/STRA = rev | | | | | | | |
| 18 | 95415_f_at | Cluster Incl AI132585 ue97d10 x1 Mus musculus cDNA, 3 end/clone = IMAGE-1499059/clone_end = 3/gb = AI132585/ | 456.8 | P | 472.9 | P | 522.1 | P | 411.5 |
| | | gi = 3602592/ug = Mm.24276/len = 597/STRA = for | | | | | | | |
| 18 | 96610_at | Cluster Incl AW046442 UI-M-BH1-akp-e-03-0-UI s1 Mus musculus cDNA, 3 end/clone = UI-M-BH1-akp-e-03-0-UI/ | 575.3 | P | 588.3 | P | 624.5 | P | 584.3 |
| | | clone_end = 3/gb = AW046442/gi = 5906971/ug = Mm.27082/len = 451/STRA = for | | | | | | | |
| 18 | 96625_at | Cluster Incl AW123132 UI-M-BH2 1-apd-a-04-0-UI s1 Mus musculus cDNA, 3 end/clone = UI-M-BH2 1-apd-a-04-0-UI/ | 330.1 | P | 339.8 | P | 317.6 | P | 301.2 |
| | | clone_end = 3/gb = AW123132/gi = 6098882/ug = Mm 2718/len = 488/STRA = for | | | | | | | |
| 18 | 96669_at | Cluster Incl AW122973 UI-M-BH2.1-apb-a-02-0-UI s1 Mus musculus cDNA, 3 end/clone = UI-M-BH2 1-apb-a-02-0-UI/ | 365.7 | P | 363.2 | P | 396.1 | P | 398.9 |
| | | clone_end = 3/gb = AW122973/gi = 6098503/ug = Mm 27394/len = 467/STRA = for | | | | | | | |
| 18 | 96302_at | Cluster Incl AA711516 vu26g06 r1 Mus musculus cDNA, 5 end/clone = IMAGE-1181818/clone_end = 5/gb = AA711516/ | 370.1 | P | 110.3 | P | 188.1 | P | 463.5 |
| | | gi = 2721434/ug = Mm 29915/len = 260/STRA = rev | | | | | | | |
| 18 | 96879_at | Cluster Incl AI852338 UI-M-BH0-ajo-a-12-0-UI s1 Mus musculus cDNA, 3 end/clone = UI-M-BH0-ajo-a-12-0-UI/ | 1045.2 | P | 1383.9 | P | 1279.3 | P | 1346.9 |
| | | clone_end = 3/gb = AI852338/gi = 5496244/ug = Mm.30074/len = 475/STRA = for | | | | | | | |
| 18 | 97442_at | Cluster Incl AW124340 UI-M-BH2 1-apq-f-11-0-UI s1 Mus musculus cDNA, 3 end/clone = UI-M-BH2 1-apq-f-11-0-UI/ | 750.3 | P | 481.8 | P | 500.6 | P | 615.2 |
| | | clone_end = 3/gb = AW124340/gi = 6099870/ug = Mm 30239/len = 224/STRA = for | | | | | | | |
| 18 | 98055_at | Cluster Incl AW121500 UI-M-BH2.2-aop-a-10-0-UI s1 Mus musculus cDNA, 3 end/clone = UI-M-BH2 2-aop-a-10-0-UI/ | 340.9 | P | 270 | P | 288 | P | 302.8 |
| | | clone_end = 3/gb = AW121500/gi = 6096833/ug = Mm 34330/len = 384/STRA = for | | | | | | | |
| 18 | 98150_at | Cluster Incl L26528 RAB11B, member RAS oncogene family/cds = (35,691)/gb = L26528/gi = 433063/ug = Mm 35727/ | 372.1 | P | 436.8 | P | 606 | P | 406.2 |
| | | len = 1763/STRA = for | | | | | | | |
| 18 | 98151_s_at | Cluster Incl Z17804 Catenin src/cds = (342,3077)/gb = Z17804/gi = 53544/ug = Mm 35738/len = 4636/STRA = rev | 619.9 | P | 545.1 | P | 497.4 | P | 614.2 |
| 18 | 99119_at | Cluster Incl D00472 Cofilin 1, non-muscle/cds = (94,594)/gb = D00472/gi = 220383/ug = Mm 4024/len = 1134/STRA = for | 3606.1 | P | 3437.1 | P | 3268.8 | P | 4117.6 |
| 18 | 99639_at | Cluster Incl D84096 Mouse mRNA for ubiquitin carboxyl-terminal hydrolase (UBP), complete cds/cds = (67,2448)/ | 337 | P | 276.4 | P | 363.1 | P | 230.5 |
| | | gb = D84096/gi = 1902990/ug = Mm 4355/len = 3236/STRA = for | | | | | | | |
| 18 | 99650_at | Cluster Incl X90945 M. musculus mRNA for casein kinase I-alpha/cds = (0,85)/gb = X90945/gi = 975690/ug = Mm.43737/ | 1712.7 | P | 1585.9 | P | 1868.1 | P | 1887.3 |
| | | len = 342/STRA = for | | | | | | | |
| 18 | 100069_at | Cluster Incl M77497 Cytochrome P450, 2f2/cds = (61,1536)/gb = M77497/gi = 192927/ug = Mm 4515/len = 1810/STRA = for | 480.2 | P | 340.4 | P | 439.9 | P | 409.6 |
| 18 | 100553_at | Cluster Incl L46855 Ret finger protein/cds = (148,1716)/gb = L46855/gi = 1161121/ug = Mm 55/len = 1950/STRA = for | 693.8 | P | 618.5 | P | 590.5 | P | 569.4 |
| 18 | 100616_at | Cluster Incl AF012710.Mus musculus centromere protein A (Cenp-a) gene, promoter region and partial cds/cds = (148,210)/ | 420.8 | P | 356.8 | P | 509.3 | P | 394.4 |
| | | gb = AF012710/gi = 2465204/ug = Mm.6579/len = 211/STRA = for | | | | | | | |
| 18 | 101050_at | Cluster Incl AF031381 Mus musculus KOI-4 gene, partial cds/cds = (0,130)/gb = AF031381/gi = 2623677/ug = Mm.7013/ | 716.3 | P | 572 | P | 849.3 | P | 640.9 |
| | | len = 341/STRA = for | | | | | | | |
| 18 | 101073_at | Cluster Incl X67469 Low density lipoprotein receptor related protein/cds = (441,14078)/gb = X67469/gi = 49941/ug = Mm 7221/ | 887.3 | P | 968.1 | P | 1005 | P | 1036.1 |
| | | len = 14849/STRA = for | | | | | | | |
| 18 | 101536_at | Cluster Incl U35312 Mus musculus nuclear receptor co-repressor mRNA, complete cds/cds = (116,7477)/gb = U35312/ | 359.8 | P | 297.8 | P | 293.1 | P | 235.9 |
| | | gi = 1022717/ug = Mm 88061/len = 7780/STRA = for | | | | | | | |
| 18 | 101555_at | Cluster Incl X57277 RAS-related C3 botulinum substrate 1/cds = (140,718)/gb = X57277/gi = 53885/ug = Mm 889/len = 886/ | 3269.7 | P | 3336.5 | P | 3481.5 | P | 3579.5 |
| | | STRA = for | | | | | | | |
| 18 | 102333_at | Cluster Incl AF057286 Mus musculus intersectin-EH binding protein Ibp2 mRNA, partial cds/cds = (0,1530)/gb = AF057286/ | 649.2 | P | 821.5 | P | 605.9 | P | 814 |
| | | gi = 3063648/ug = Mm 10754/len = 2322/STRA = for | | | | | | | |
| 18 | 102767_at | Cluster Incl AW121700 UI-M-BH2 3-aoc-g-01-0-UI s1 Mus musculus cDNA, 3 end/clone = UI-M-BH2,3-aoc-g-01-0-UI/ | 1102.3 | P | 1082.4 | P | 1088 | P | 1114.8 |
| | | clone_end = 3/gb = AW121700/gi = 6097033/ug = Mm 13080/len = 387/STRA = for | | | | | | | |
| 18 | 103020_s_at | Cluster Incl AI317205 ui35g09 y1 Mus musculus cDNA, 5 end/clone = IMAGE-1853344/clone_end = 5/gb = AI317205/ | 511.2 | P | 384.6 | P | 152.2 | P | 502.9 |
| | | gi = 4032472/ug = Mm 15918/len = 235/STRA = for | | | | | | | |
| 18 | 103029_at | Cluster Incl D86344 Mouse mRNA for Topoisomerase-lnhibitor suppressed, complete cds/cds = (86,1495)/gb = D86344/ | 991.7 | P | 877 4 | P | 811.6 | P | 757.5 |
| | | gi = 1711237/ug = Mm 1605/len = 2196/STRA = for | | | | | | | |
| 18 | 103073_i_at | Cluster Incl AI842969.UI-M-AO1-aem-g-05-0-UI s1 Mus musculus cDNA, 3 end/clone = UI-M-AO1-aem-g-05-0-UI/ | 871.7 | P | 996.3 | P | 798.6 | P | 865.1 |
| | | clone_end = 3/gb = AI842969/gi = 5477182/ug = Mm 16976/len = 340/STRA = for | | | | | | | |
| 18 | 103099_f_at | Cluster Incl AI663971 ui16h11 r1 Mus musculus cDNA, 5 end/clone = IMAGE-1851525/clone_end = 5/gb = AI663971/ | 1289.6 | P | 1185.2 | P | 1273.2 | P | 1287.3 |
| | | gi = 4767554/ug = Mm 17537/len = 400/STRA = for | | | | | | | |
| 18 | 103435_at | Cluster Incl AW045910 UI-M-BH1-aku-c-03-0-UI s1 Mus musculus cDNA, 3 end/clone = UI-M-BH1-aku-c-03-0-UI/ | 958.2 | P | 887.2 | P | 923.4 | P | 1134.3 |
| | | clone_end = 3/gb = AW045910/gi = 5906439/ug = Mm 19073/len = 454/STRA = for | | | | | | | |
| 18 | 103597_at | Cluster Incl X63349 M musculus tyrp2 mRNA for tyrosinase-related protein-2/cds = (404,1957)/gb = X63349/gi = 55065/ | 1184.6 | P | 961 | P | 924.3 | P | 966.4 |
| | | ug = Mm 19987/len = 2182/STRA = for | | | | | | | |
| 18 | 103810_at | Cluster Incl AB010152 Mus musculus mRNA for p73H, complete cds/cds = (118,1878)/gb = AB010152/gi = 3445481/ | 898.7 | P | 909.8 | P | 789.1 | P | 889.1 |
| | | ug = Mm 20894/len = 4669/STRA = for | | | | | | | |
| 18 | 103816_at | Cluster Incl U89915: Mus musculus junctional adhesion molecule (Jam) mRNA, complete cds/cds = (70,972)/gb = U89915/ | 802.5 | P | 708.2 | P | 963.7 | P | 872.9 |
| | | gi = 3462454/ug = Mm.20903/len = 2029/STRA = for | | | | | | | |
| 18 | 103855_at | Cluster Incl AW123286 UI-M-BH2 1-apg-g-06-0-UI S1 Mus musculus cDNA, 3 end/clone = UI-M-BH2 1-apg-g-06-0-UI/ | 540 | P | 615.5 | P | 659 | P | 665 |
| | | clone_end = 3/gb = AW123286/gi = 6098816/ug = Mm 21072/len = 368/STRA = for | | | | | | | |
| 18 | 104625_at | Cluster Incl AA874130 vw87g02 r1 Mus musculus cDNA, 5 end/clone = IMAGE-1261970/clone_end = 5/gb = AA874130/ | 538.4 | P | 650.3 | P | 650.9 | P | 516.7 |
| | | gi = 2978819/ug = Mm 24265/len = 204/STRA = for | | | | | | | |
| 18 | 94375_at | Cluster Incl Y11666 M musculus gene encoding hexokinase II, exon 1 (and joined CDS)/cds = (479,3232)/gb = Y11666/ | 274.3 | P | 304.4 | P | 272 | P | 327.4 |
| | | gi = 1907082/ug = Mm.2549/len = 5496/STRA = for | | | | | | | |
| 18 | 96185_at | Cluster Incl AB004305 Adaptor-related protein complex AP-3, delta subunit/cds = (220,3819)/gb = AB004305/gi = 2828340 | 811.8 | P | 972.9 | P | 893.6 | P | 843.1 |
| | | ug = Mm 28463/len = 4730/STRA = rev | | | | | | | |
| 18 | 96818_at | Cluster Incl AA762522 vv84d02 r1 Mus musculus cDNA, 5 end/clone = IMAGE-1229091/clone_end = 5/gb = AA762522/ | 418.9 | P | 370.4 | P | 387.7 | P | 334.5 |
| | | gi = 2812269/ug = Mm.29343/len = 523/STRA = for | | | | | | | |
| 18 | 97823_at | Cluster Incl AW046532 UI-M-BH1-akz-g-10-0-UI s1 Mus musculus cDNA, 3 end/clone = UI-M-BH1-akz-g-10-0-UI/ | 499 | P | 525.2 | P | 501.2 | P | 494.7 |
| | | clone/end = 3/gb = AW046532/gi = 5907061/ug = Mm 30167/len = 509/STRA = for | | | | | | | |
| 18 | 97933_at | Cluster Incl AW045317: UI-M-BH1-akn-g-06-0-UI.s1 Mus musculus cDNA, 3 end/clone = UI-M-BH1-akn-g-06-0-UI/ | 754.7 | P | 549.2 | P | 542.9 | P | 770.6 |
| | | clone_end = 3/gb = AW045317/gi = 5905846/ug = Mm.30248/len = 324/STRA = for | | | | | | | |
| 18 | 97951_s_at | Cluster Incl U37775 Tuberous sclerosis 2/cds = (34,5259)/gb = U37775/gi = 1181664/ug = Mm 30435/len = 5351/STRA = for | 222.5 | P | 182.6 | P | 400.9 | P | 396.8 |
| 18 | 97989_at | Cluster Incl M81483 Mus musculus calmodulin dependent phosphatase catalytic subunit (Cam-Prp) mRNA, 3 end/ | 313.1 | P | 235 | P | 199.2 | P | 264.2 |
| | | cds = (0,1547)/gb = M81483/gi = 192537/ug = Mm.3150/len = 1800/STRA = rev | | | | | | | |
| 18 | 98033_at | Cluster Incl AA710132: vt45a05 r1 Mus musculus cDNA, 5 end/clone = IMAGE-1166000/clone_end = 5/gb = AA710132/ | 1162.8 | P | 1208.4 | P | 1185.1 | P | 1299.7 |
| | | gi = 2720050/ug = Mm 3311/len = 339/STRA = for | | | | | | | |
| 18 | 98424_at | Cluster Incl D83966 Protein tyrosine phosphatase, non-receptor type 13/cds = (41,7393)/gb = D83966/gi = 1232103/ | 555.2 | P | 473.2 | P | 510.9 | P | 535.6 |
| | | ug = Mm 3414/len = 7912/STRA = rev | | | | | | | |
| 18 | 98975_at | Cluster Incl AI019999 ua95g09 r1 Mus musculus cDNA, 5 end/clone = IMAGE-1365280/clone_end = 5/gb = AI019999/ | 336 | P | 303 | P | 312.2 | P | 294.1 |
| | | gi = 3234335/ug = Mm 36725/len = 486/STRA = for | | | | | | | |
| 18 | 99025_at | Cluster Incl L25125 Mouse RNA helicase and RNA-dependent ATPase from the DEAD box family mRNA, complete cds/ | 455.3 | P | 309.7 | P | 542.8 | P | 703.4 |
| | | cds = (97,1533)/gb = L25125/gi = 407991/ug = Mm 3880/len = 2552/STRA = rev | | | | | | | |
| 18 | 99920_at | Cluster Incl U65586: Telomeric repeat binding factor 1/cds = (12,1277)/gb = U65586/gi = 1655898/ug = Mm 4306/len = 1628/ | 105.9 | P | 31.3 | P | 122 | P | 80.3 |
| | | STRA = rev | | | | | | | |
| 18 | 100887_at | Cluster Incl AI838562 UI-M-AO0-abz-a-04-0-UI s1 Mus musculus cDNA, 3 end/clone = UI-M-AO0-abz-a-04-0-UI/ | 298.2 | P | 156 | P | 134.6 | P | 224.8 |
| | | clone_end = 3/gb = AI838562/gi = 5472775/ug = Mm 5383/len = 252/STRA = for | | | | | | | |
| 18 | 100889_at | Cluster Incl AI838576 UI-M-AO0-abz-c-02-0-UI s1 Mus musculus cDNA, 3 end/clone = UI-M-AO0-abz-c-02-0-UI/ | 555.7 | P | 439.4 | P | 672.4 | P | 638.5 |
| | | clone_end = 3/gb = AI838576/gi = 5472789/ug = Mm 54120/len = 161/STRA = rev | | | | | | | |
| 18 | 100915_at | Cluster Incl AW125698 UI-M-BH2 2-aqi-f-10-0-UI s1 Mus musculus cDNA, 3 end/clone = UI-M-BH2 2-aqi-f-10-0-UI/ | 2403.9 | P | 2889.8 | P | 2961.1 | P | 3300.5 |
| | | clone_end = 3/gb = AW125698/gi = 6101228/ug = Mm.5938/len = 128/STRA = for | | | | | | | |
| 18 | 101389_at | Cluster Incl AB008553 Mus musculus mRNA for mLGP85/LIMP II, complete cds/cds = (205,1641)/gb = AB008553/ | 332.4 | P | 237.5 | P | 302.5 | P | 350.5 |
| | | gi = 2618485/ug = Mm.7176/len = 1853/STRA = for | | | | | | | |
| 18 | 101851_at | Cluster Incl AF029215 Antigen Identified by monoclonal antibody MRC OX-2/cds = (0,836)/gb = AF029215/gi = 2739125/ | 325 | P | 355.7 | P | 320.5 | P | 351.2 |
| | | ug = Mm 8649/len = 837/STRA = for | | | | | | | |
| 18 | 102211_r_at | Cluster Incl U66835: Mus musculus unknown protein mRNA, partial cds/cds = (0,98)/gb = U66835/gi = 2970116/ | 249.1 | P | 218.6 | P | 268.6 | P | 298.2 |
| | | ug = Mm 10102/len = 323/STRA = for | | | | | | | |
| 18 | 102670_at | Cluster Incl AI848706 UI-M-AH1-agv-h-09-0-UI s1 Mus musculus cDNA, 3 end/clone = UI-M-AH1-agv-b-09-0-UI/ | 379.2 | P | 201.2 | P | 338.5 | P | 296.6 |
| | | clone_end = 3/gb = AI848706/gi = 5492612/ug = Mm.13758/len = 542/STRA = for | | | | | | | |
| 18 | 102930_at | Cluster Incl U14133: Silver/cds = (0,1880)/gb = U14133/gi = 887940/ug = Mm 16756/len = 1881/STRA = for | 1084.1 | P | 965 | P | 856.4 | P | 1012.7 |
| 18 | 103443_at | Cluster Incl AA711704 vu58c09 r1 Mus musculus cDNA, 5 end/clone = IMAGE-1195600/clone_end = 5/gb = AA711704/ | 1746.6 | P | 1349.2 | P | 1779.4 | P | 1714 |
| | | gi = 2721622/ug = Mm.21476/len = 249/STRA = for | | | | | | | |
| 18 | 103783_at | Cluster Incl AI648965 uk33d12.x1 Mus musculus cDNA, 3 end/clone = lMAGE-1970807/clone_end = 3/gb = AI648965/ | 204.6 | P | 386.6 | P | 386.6 | P | 225.9 |
| | | gi = 4729799/ug = Mm.24893/len = 509/STRA = rev | | | | | | | |
| 18 | 104244_at | Cluster Incl AI606891 vw20f11 x1 Mus musculus cDNA, 3 end/clone = IMAGE-1244397/clone_end = 3/gb = AI606891/ | 343.8 | P | 319.6 | P | 381.7 | P | 360.2 |
| | | gi = 4616058/ug = Mm 28210/len = 322/STRA = for | | | | | | | |
| 18 | 104248_at | Cluster Incl AW227650 up12b09 y1 Mus musculus cDNA, 5 end/clone = IMAGE-2654105/clone_end = 5/gb = AW227650/ | 835.9 | P | 581.2 | P | 726.1 | P | 754.5 |
| | | gi = 6556946/ug = Mm 28345/len = 440/STRA = for | | | | | | | |
| 18 | 104437_at | Cluster Incl Z30174: Zinc finger protein 30/cds = (143,1789)/gb = Z30174/gi = 456268/ug = Mm 2927/len = 2604/STRA = for | 359.6 | P | 351 | P | 499.7 | P | 536.6 |
| 18 | 92262_at | Cluster Incl AF012923 Mus musculus p53-inducible zinc finger protein (Wig-1) mRNA, complete cds/cds = (173,1045)/ | 301 | P | 276.6 | P | 212.4 | P | 370.1 |
| | | gb = AF012923/gi = 2804680/ug = Mm 35705/len = 7661/STRA = for | | | | | | | |
| 18 | 92394_f_at | Cluster Incl AB010266 Mus musculus Creb-rp, Tnx and Cyp21 genes for cAMP response element binding protein-related | 1048 | P | 549.7 | P | 952.3 | P | 944.6 |
| | | protein, tenascin-X and steroid 21-hydroxylase, partial cds and complete sequences/cds = (0,142)/gb = AB010266/ | | | | | | | |
| | | gi = 2804287/ug = Mm 4068/len = 515/STRA = for | | | | | | | |
| 18 | 92440_at | Cluster Incl U73029 Mus musculus interferon regulatory factor 6 (mirf6) mRNA, complete cds/cds = (141,1544)/gb = U73029/ | 1004.7 | P | 817.5 | P | 771.2 | P | 959.4 |
| | | gi = 1685127/ug = Mm 4179/len = 4046/STRA = rev | | | | | | | |
| 18 | 92915_s_at | Cluster Incl X13961 Mouse rearranged homeotic gene Hox-2 4 (IAP Integration)/cds = (0,731)/gb = X13961/gi = 51393/ | 420.1 | P | 572.7 | P | 571.4 | P | 508.5 |
| | | ug = Mm 4822/len = 732/STRA = for | | | | | | | |
| 18 | 92922_at | Cluster Incl U17291: Mus musculus transcription factor AP-2 (AP-2) gene, alternative/cds = (36,602)/gb = U17291/ | 876.2 | P | 868.8 | P | 941.8 | P | 913.4 |
| | | gi = 755199/ug = Mm 4845/len = 603/STRA = for | | | | | | | |
| 18 | 93454_at | Cluster Incl AF081789 Mus musculus cell surface antigen AA4 (AA4) mRNA, complete cds/cds = (120,2054)/gb = AF081789/ | 370.8 | P | 414.7 | P | 475.1 | P | 380.7 |
| | | gi = 3695112/ug = Mm 681/len = 3070/STRA = for | | | | | | | |
| 18 | 93875_at | Cluster Incl M12571: Heat shock protein, 70 kDa 3/cds = (0,1262)/gb = M12571/gi = 194014/ug = Mm 88432/len = 1506/ | 608.1 | P | 553.2 | P | 667.4 | P | 525.3 |
| | | STRA = rev | | | | | | | |
| 18 | 96506_at | Cluster Incl D83002: Anaplastic lymphoma kinase/cds = (450,5315)/gb = D83002/gi = 1864006/ug = Mm.2536/len = 5767/ | 339.9 | P | 313 | P | 362.5 | P | 324.7 |
| | | STRA = for | | | | | | | |
| 18 | 96530_at | Cluster Incl AI503926 vm33f03 x1 Mus musculus cDNA, 3 end/clone = IMAGE-991997/clone_end = 3/gb = AI503926/ | 202.9 | P | 95.6 | P | 202 | P | 175.4 |
| | | gi = 4401777/ug = Mm 27091/len = 342/STRA = for | | | | | | | |
| 18 | 97722_at | Cluster Incl AA879709 vv97e03 r1 Mus musculus cDNA, 5 end/clone = IMAGE-1230364/clone_end = 5/gb = AA879709/ | 627.9 | P | 415.9 | P | 549.3 | P | 527.5 |
| | | gi = 2988692/ug = Mm.39052/len = 404/STRA = for | | | | | | | |
| 18 | 97738_r_at | Cluster Incl AW121931 UI-M-BH2 3-aoj-e-01-0-UI s1 Mus musculus cDNA, 3 end/clone = UI-M-BH2.3-aoj-e-01-0-UI/ | 398.3 | P | 250.8 | P | 441.5 | P | 382.6 |
| | | clone_end = 3/gb = AW121931/gi = 6097377/ug = Mm 3957/len = 367/STRA = rev | | | | | | | |
| 18 | 98308_at | Cluster Incl AJ002522.Mus musculus mRNA for myosin heavy chain 2X, partial/cds = (0,94)/gb = AJ002522/gi = 4582679/ | 703.2 | P | 864.9 | P | 415.4 | P | 1327.5 |
| | | ug = Mm 42156/len = 199/STRA = rev | | | | | | | |
| 18 | 100357_g_at | Cluster Incl AW121051 UI-M-BH2 3-aof-d-02-0-UI s1 Mus musculus cDNA, 3 end/clone = UI-M-BH2 3-aof-d-02-0-UI/ | 478.1 | P | 434.5 | P | 402.8 | P | 337.2 |
| | | clone_end = 3/gb = AW121051/gi = 6096384/ug = Mm.88761/len = 365/STRA = for | | | | | | | |
| 18 | 101291_at | Cluster Incl Z83956 M musculus spermidine synthase pseudogene/cds = (0,894)/gb = Z83956/gi = 1781038/ug = Mm 56916/ | 314.2 | P | 286.7 | P | 253.3 | P | 281.3 |
| | | len = 895/STRA = for | | | | | | | |
| 18 | 101795_f_at | Cluster Incl U12566 Defensin related cryptdin, related sequence 12/cds = (0,278)/gb = U12566/gi = 531852/ug = Mm 57135/ | 725.3 | P | 926.7 | P | 921.5 | P | 926.3 |
| | | len = 279/STRA = for | | | | | | | |
| 18 | 96375_at | Cluster Incl C80249: C80249 Mus musculus cDNA, 3 end/clone = J0078F09/clone_end = 3/gb = C80249/gi = 2520579/ | 531 | P | 407.3 | P | 330.4 | P | 367.5 |
| | | ug = Mm.25065/len = 609/STRA = rev | | | | | | | |
| 18 | 102105_f_at | Cluster Incl AI840733: UI-M-AM0-adn-b-04-0-UI s1 Mus musculus cDNA, 3 end/clone = UI-M-AM0-adn-b-04-0-UI/ | 341.1 | P | 211.4 | P | 211.8 | P | 259.6 |
| | | clone_end = 3/gb = AI840733/gi = 5474946/ug = Mm 42506/len = 275/STRA = rev | | | | | | | |
| 18 | 96087_at | Cluster Incl L07037 Mannoside acetylglucosaminyl transferase 1/cds = (363,1706)/gb = L07037/gi = 202145/ug = Mm 2672/ | 798.2 | P | 649.1 | P | 769.2 | P | 638.9 |
| | | len = 2695/STRA = for | | | | | | | |
| 18 | 95679_at | Cluster Incl AI835912.UI-M-AI0-aao-a-07-0-UI s1 Mus musculus cDNA, 3 end/clone = UI-M-AI0-aao-a-07-0-UI/ | 600 | P | 455.7 | P | 585.8 | P | 461.3 |
| | | clone_end = 3/gb = AI835912/gi = 5470120/ug = Mm 29609/len = 455/STRA = for | | | | | | | |
| 18 | 97546_at | Cluster Incl AF072127 Mus musculus claudin-1 mRNA, complete cds/cds = (155,790)/gb = AF072127/gi = 3335181/ | 301.2 | P | 103.3 | P | 217.2 | P | 210.3 |
| | | ug = Mm 33669/len = 952/STRA = for | | | | | | | |
| 18 | 102750_at | Cluster Incl AF070975 Mus musculus X11 gamma protein mRNA, complete cds/cds = (218,1933)/gb = AF070975/ | 408.3 | P | 423.4 | P | 356.2 | P | 431.3 |
| | | gi = 3264793/ug = Mm 12909/len = 2039/STRA = for | | | | | | | |
| 18 | 103629_g_at | Cluster Incl D16503.Lymphoid enhancer binding factor 1/cds = (1804,2658)/gb = D16503/gi = 391762/ug = Mm 2029/ | 231.7 | P | 201.3 | P | 117.8 | M | 163.6 |
| | | len = 2740/STRA = rev | | | | | | | |
| 18 | 103900_at | Cluster Incl AW124150 UI-M-BH2 1-apr-f-07-0-UI s1 Mus musculus cDNA, 3 end/clone = UI-M-BH2 1-apr-f-07-0-UI/ | 356.1 | P | 359.4 | P | 328.6 | P | 173.8 |
| | | clone_end = 3/gb = AW124150/gi = 6099680/ug = Mm 2133/len = 254/STRA = for | | | | | | | |
| 18 | 95533_at | Cluster Incl AW048037 UI-M-BH1-alv-c-01-0-UI.s1 Mus musculus cDNA, 3 end/clone = UI-M-BH1-alv-c-01-0-UI/ | 180.1 | P | 289.6 | P | 277.5 | P | 371.8 |
| | | clone_end = 3/gb = AW048037/gi = 5908531/ug = Mm.27653/len = 396/STRA = for | | | | | | | |
| 18 | 97955_at | Cluster Incl X59513 Mouse 5 end of TRP1 gene for tyrosinase-related protein-1/cds = (185,187)/gb = X59513/gi = 54913/ | 176.1 | P | 123.9 | P | 144.5 | P | 130.4 |
| | | ug = Mm 30438/len = 297/STRA = for | | | | | | | |
| 18 | 98490_at | Cluster Incl AA822412: vw37d10r1 Mus musculus cDNA, 5 end/clone = IMAGE-1246003/clone_end = 5/gb = AA822412/ | 167.7 | P | 91.3 | P | 108.4 | P | 131.4 |
| | | gi = 2892280/ug = Mm 35596/len = 460/STRA = for | | | | | | | |
| 18 | 99049_at | Cluster Incl D28492: Caspase 2/cds = (6,1364)/gb = D28492/gi = 3090429/ug = Mm 3921/len = 3395/STRA = for | 709 | P | 605.1 | P | 687.9 | P | 507.3 |
| 18 | 102230_at | Cluster Incl AF011567: Hyaluronidase 1/cds = (53,1441)/gb = AF011567/gi = 2961469/ug = Mm 10305/len = 2125/STRA = for | 144.5 | P | 188.2 | A | 207.6 | P | 223.4 |
| 18 | 95957_at | Cluster Incl C77070 C77070 Mus musculus cDNA, 3 end/clone = J0025F02/clone_end = 3/gb = C77070/gi = 2517400/ | 290.4 | P | 289.6 | P | 193.9 | P | 230.1 |
| | | ug = Mm 24934/len = 592/STRA = rev | | | | | | | |
| 18 | 99114_r_at | Cluster Incl AW045905 UI-M-BH1-aku-b-09-0-UI s1 Mus musculus cDNA, 3 end/clone = UI-M-BH1-aku-b-09-0-UI/ | 201.7 | P | 168.7 | P | 229.5 | A | 150.6 |
| | | clone_end = 3/gb = AW045905/gi = 5906434/ug = Mm 400/len = 464/STRA = for | | | | | | | |
| 18 | 102787_at | Cluster Incl AI841654 UI-M-AO0-acg-e-05-0-UI s1 Mus musculus cDNA, 3 end/clone = UI-M-AO0-acg-e-05-0-UI/ | 346.4 | P | 148.7 | P | 277.7 | P | 369.1 |
| | | clone_end = 3/gb = AI841654/gi = 5475867/ug = Mm 13799/len = 284/STRA = for | | | | | | | |
| 18 | 103668_at | Cluster Incl U40375 Suppressor of Ty 6 (S cerevisiae) homolog/cds = (184,5364)/gb = U40375/gi = 1401056/ug = Mm 20755/ | 350.5 | M | 319 | P | 359.2 | P | 318.2 |
| | | len = 5923/STRA = for | | | | | | | |
| 18 | 104534_at | Cluster Incl AA623874 vq73h02 s1 Mus musculus cDNA, 5 end/clone = IMAGE-1107987/clone_end = 5/gb = AA623874/ | 90.4 | P | 58.7 | P | 66.9 | P | 59.2 |
| | | gi = 2527750/ug = Mm 2325/len = 318/STRA = for | | | | | | | |
| 18 | 99956_at | Cluster Incl Y00864 Mouse c-kit mRNA/cds = (28,2955)/gb = Y00864/gi = 50423/ug = Mm 4394/len = 5098/STRA = for | 126.8 | P | 121.3 | P | 157.7 | P | 184.1 |
| 18 | 103281_at | Cluster Incl AF077003: Mus musculus SH3 domain-containing adapter protein mRNA, complete cds/cds = (6,1931)/ | 199.5 | P | 98.1 | P | 49.7 | P | 125.2 |
| | | gb = AF077003/gi = 3550239/ug = Mm 20882/len = 2431/STRA = rev | | | | | | | |
| 18 | 92196_f_at | Cluster Incl X83733 M musculus SAP62-AMH gene/cds = (0,1457)/gb = X83733/gi = 633250/ug = Mm 34039/len = 1458/ | 1069 | P | 938.7 | P | 1108.9 | P | 597.5 |
| | | STRA = for | | | | | | | |
| 18 | 93475_at | Cluster Incl AI845581 UI-M-AQ1-adx-c-02-0-UI s1 Mus musculus cDNA, 3 end/clone = UI-M-AQ1-adx-c-02-0-UI/ | 158.5 | P | 111.2 | A | 89.7 | P | 154.2 |
| | | clone_end = 3/gb = AI845581/gi = 5489487/ug = Mm 7086/len = 136/STRA = rev | | | | | | | |
| 18 | 99370_at | Cluster Incl AF095447 Mus musculus natural killer cell receptor NKG2A (Nkg2a) mRNA, complete cds/cds = (0,734)/ | 204.4 | P | 192.7 | P | 173.1 | P | 149.2 |
| | | gb = AF095447/gi = 4115906/ug = Mm 56899/len = 735/STRA = for | | | | | | | |
| 18 | 100385_at | Cluster Incl X75832 M musculus Glra1 gene (exon1)/cds = (0,1349)/gb = X75832/gi = 435518/ug = Mm.89320/len = 1350/ | 443.1 | P | 385.3 | P | 342.4 | P | 354.8 |
| | | STRA = rev | | | | | | | |
| 18 | 94622_at | Cluster Incl U17252: Mus musculus metabolropic glutamate receptor 8 (mGluR8) mRNA, complete cds/cds = (70,2796)/ | 226.7 | P | 146.1 | P | 178.2 | P | 210.6 |
| | | gb = U17252/gi = 854728/ug = Mm 8001/len = 2830/STRA = for | | | | | | | |
| 18 | 99777_s_at | Cluster Incl C76650 C76650 Mus musculus cDNA, 3 end/clone = J0015G08/clone_end = 3/gb = C76650/gi = 2516980/ | 68.1 | P | 75.4 | P | 55.5 | P | 16.1 |
| | | ug = Mm 97081/len = 611/STRA = rev | | | | | | | |
| 18 | 95155_at | Cluster Incl AI853875 UI-M-BH0-ajr-g-11-0-UI.s1 Mus musculus cDNA, 3 end/clone = UI-M-BH0-ajr-g-11-0-UI/ | 245 | P | 220 | P | 338.6 | P | 249.8 |
| | | clone_end = 3/gb = AI853875/gi = 5497781/ug = Mm 29479/len = 416/STRA = for | | | | | | | |
| 18 | 101522_at | Cluster Incl L15429 Mus musculus L6 antigen mRNA, complete cds/cds = (91,699)/gb = L15429/gi = 468909/ug = Mm 856/ | 58.2 | P | 32.2 | P | 27.3 | A | 85.6 |
| | | len = 1077/STRA = for | | | | | | | |
| 18 | 97397_at | Cluster Incl AI848344 UI-M-AH1-ago-d-01-0-UI s1 Mus musculus cDNA, 3 end/clone = UI-M-AH1-ago-d-01-0-UI/ | 97.1 | A | 136.8 | P | 139.8 | P | 149.9 |
| | | clone_end = 3/gb = AI848344/gi = 5492250/ug = Mm 29849/len = 404/STRA = for | | | | | | | |
| 18 | 100491_at | Cluster Incl AF045692 Solute carrier family 16 (monocarboxylic acid transporters), member 2/cds = (174,1871)/ | 61 | A | 94.9 | M | 102.6 | P | 82.7 |
| | | gb = AF045692/gi = 2944355/ug = Mm 5045/len = 4247/STRA = for | | | | | | | |
| 18 | 102228_at | Cluster Incl AF036907: Mus musculus linker for activation of T cells (LAT) mRNA, complete cds/cds = (291,1019)/ | 856.8 | P | 837.8 | P | 672.4 | P | 671.3 |
| | | gb = AF036907/gi = 2828027/ug = Mm 10280/len = 1260/STRA = for | | | | | | | |
| 18 | 103303_at | Cluster Incl AA798246: vy05e06.r1 Mus musculus cDNA, 5 end/clone = IMAGE-1294594/clone_end = 5/gb = AA798246/ | 69.9 | P | 94.2 | A | 87.3 | P | 86.1 |
| | | gi = 2861201/ug = Mm 21099/len = 494/STRA = for | | | | | | | |
| 18 | 92303_at | Cluster Incl D31898: Protein tyrosine phosphatase, receptor type, R/cds = (355,2325)/gb = D31898/gi = 695408/ug = Mm.3771/ | 915.9 | P | 719.3 | P | 605.8 | A | 865.3 |
| | | len = 3430/STRA = for | | | | | | | |
| 18 | 94197_at | Cluster Incl D89866: Mus musculus mRNA for ceramide glucosyltransferase, complete cds/cds = (185,1369)/gb = D89866/ | 115.4 | A | −21.7 | A | −15.5 | P | 163.7 |
| | | gi = 3219161/ug = Mm.12971/len = 1523/STRA = for | | | | | | | |
| 18 | 94733_at | Cluster Incl J03398 P glycoprotein 2/cds = (193,4023)/gb = J03398/gi = 199109/ug = Mm 14172/len = 4084/STRA = for | 335.9 | A | 361.1 | P | 316.5 | P | 311.1 |
| 18 | 98846_f_at | Cluster Incl M83344 Carcinoembryonic antigen 2/cds = (192,1619)/gb = M83344/gi = 200316/ug = Mm 5056/len = 2043/ | 957.1 | P | 597.9 | M | 701.2 | P | 658.6 |
| | | STRA = for | | | | | | | |
| 18 | 101229_at | Cluster Incl C78228: C78228 Mus musculus cDNA, 3 end/clone = J0044F11/clone_end = 3/gb = C78228/gi = 2518558/ | 329.6 | A | 98.5 | P | 88.4 | P | 136.7 |
| | | ug = Mm 5692/len = 553/STRA = rev | | | | | | | |
| 18 | 93516_at | Cluster Incl M25825: T-complex lestis expressed 1/cds = (12,353)/gb = M25825/gi = 201912/ug = Mm.1948/len = 680/ | 95.2 | P | 100.1 | M | 115.4 | P | 66.9 |
| | | STRA = for | | | | | | | |
| 18 | 94211_at | Cluster Incl AA959180 ua07g06 r1 Mus musculus cDNA, 5 end/clone = IMAGE-1346074/clone_end = 5/gb = AA959180/ | 424.5 | P | 210.8 | P | 244.7 | P | 220.7 |
| | | gi = 3124373/ug = Mm 22214/len = 347/STRA = for | | | | | | | |
| 18 | 97239_at | Cluster Incl AI845106: UI-M-BG0-ahs-a-04-0-UI s1 Mus musculus cDNA, 3 end/clone = UI-M-BG0-ahs-a-04-0-UI/ | 63.9 | P | 49.8 | P | 43.4 | P | 63.3 |
| | | clone_end = 3/gb = AI845106/gi = 5489012/ug = Mm 27839/len = 141/STRA = for | | | | | | | |
| 18 | 103055_r_at | Cluster Incl M12130 RNA polymerase II 1/cds = (0,5798)/gb = M12130/gi = 200793/ug = Mm 16533/len = 5799/STRA = rev | 944.9 | P | 826.1 | A | 806.4 | A | 796.7 |
| 18 | 104079_at | Cluster Incl AW124678 UI-M-BH2 1-apw-g-03-0-UI s1 Mus musculus cDNA, 3 end/clone = UI-M-BH2 1-apw-g-03-0-UI/ | 163.7 | P | −53.9 | P | 218.6 | P | 248.1 |
| | | clone_end = 3/gb = AW124678/gi = 6100208/ug = Mm.21735/len = 406/STRA = for | | | | | | | |
| 18 | 95011_at | Cluster Incl AI853090 UI-M-BH0-ajf-b-06-0-UI s1 Mus musculus cDNA, 3 end/clone = UI-M-BH0-ajf-b-06-0-UI/ | 100.8 | P | 45 | P | 154.4 | M | 54 |
| | | clone_end = 3/gb = AI853090/gi = 5496996/ug = Mm 27434/len = 444/STRA = for | | | | | | | |
| 18 | 95606_at | Cluster Incl AF093821 Mus musculus RRM RNA binding protein GRY-RBP mRNA, complete cds/cds = (6,1883)/ | 27.7 | P | 52.6 | P | 55.7 | P | 48.5 |
| | | gb = AF093821/gi = 3694985/ug = Mm 27972/len = 2373/STRA = for | | | | | | | |
| 18 | 97368_at | Cluster Incl U95145 Mus musculus S-AKAP84 mRNA, complete cds/cds = (197,1840)/gb = U95145/gi = 2072304/ | 945 | A | 873.1 | M | 1124.4 | P | 1045.1 |
| | | ug = Mm 2969/len = 2965/STRA = for | | | | | | | |
| 18 | 97372_at | Cluster Incl AA880432: vw89f04.r1 Mus musculus cDNA, 5 end/clone = IMAGE-1262143/clone_end = 5/gb = AA880432/ | 392.4 | P | 40.4 | A | 399.3 | P | 180.8 |
| | | gi = 2989415/ug = Mm 29701/len = 200/STRA = for | | | | | | | |
| 18 | 99523_at | Cluster Incl AA684502 vr02a04 r1 Mus musculus cDNA, 5 end/clone = IMAGE-1110606/clone_end = 5/gb = AA684502/ | 633.5 | P | 558.9 | P | 530.4 | A | 625.7 |
| | | gi = 2670219/ug = Mm 4206/len = 479/STRA = for | | | | | | | |
| 18 | 99931_at | Cluster Incl U37501 Laminin, alpha 5/cds = (0,10907)/gb = U37501/gi = 2599231/ug = Mm 4339/len = 11009/STRA = rev | 228.8 | A | 77.9 | P | 172.3 | P | 114.7 |
| 18 | 100508_at | Cluster Incl AF015769 Mus musculus manic fringe (manic-fringe) mRNA, complete cds/cds = (0,965)/gb = AF015769/ | 303.2 | P | 278.8 | M | 196.3 | P | 337.2 |
| | | gi = 2454571/ug = Mm 517/len = 966/STRA = for | | | | | | | |
| 18 | 101932_at | Cluster Incl D83484 Mouse mRNA for protein tyrosine phosphatase epsilon, complete cds/cds = (660,2759)/gb = D83484/ | 170.5 | A | 187.5 | P | 215.9 | P | 167.7 |
| | | gi = 1199932/ug = Mm 945/len = 2827/STRA = for | | | | | | | |
| 18 | 92754_at | Cluster Incl D49920 Mouse mRNA for ferredoxin-NADP reductase, complete cds/cds = (82,1566)/gb = D49920/gi = 1088468/ | 204.6 | A | 338.8 | P | 209.8 | P | 227.2 |
| | | ug = Mm 4719/len = 1866/STRA = rev | | | | | | | |
| 18 | 92936_at | Cluster Incl X14943 Contactin 1/cds = (249,3311)/gb = X14943/gi = 50937/ug = Mm 4911/len = 3843/STRA = for | 65.2 | P | 66.8 | M | 67.7 | P | 68.9 |
| 18 | 98312_at | Cluster Incl AB016768 Mus musculus mRNA for thrombospondin type 1 domain, complete cds/cds = (482,1279)/ | 210.3 | P | 125 | P | 215.1 | P | 150.8 |
| | | gb = AB016768/gi = 4519540/ug = Mm 42202/len = 1834/STRA = for | | | | | | | |
| 18 | 98385_at | Cluster Incl D31842: Mouse mRNA for putative protein tyrosine phosphatase (PTP36), complete cds/cds = (133,3702)/ | 242.5 | P | 245.9 | A | 269.3 | A | 282.5 |
| | | gb = D31842/gi = 507330/ug = Mm 4498/len = 3957/STRA = rev | | | | | | | |
| 18 | 101148_at | Cluster Incl D28577 Protein kinase C, lamda/cds = (36,1796)/gb = D28577/gi = 3452685/ug = Mm 4801/len = 1966/STRA = for | 191.9 | P | 166.7 | A | 180.2 | P | 179.9 |
| 18 | 97894_at | Cluster Incl AF109905 Mus musculus major histocompatibility locus class III regions Hsc70t gene, partial cds, smRNP, | 293.8 | P | 262.2 | A | 187.3 | A | 275.9 |
| | | G7A, NG23, Muts homolog, CLCP, NG24, NG25, and NG26 genes, complete cds, and unknown genes/cds = (0,3791)/ | | | | | | | |
| | | gb = AF109905/gi = 3986751/ug = Mm 2/STRA = for | | | | | | | |
| 18 | 94463_at | Cluster Incl X78874: Chloride channel 3/cds = (375,2657)/gb = X78874/gi = 854275/ug = Mm.28842/len = 2758/STRA = for | 38.3 | P | 46.7 | M | −13.1 | A | 58.1 |
| 18 | 102769_f_at | Cluster Incl AB016248 Mus musculus mRNA for sterol-C5-desaturase, complete cds/cds = (170,1069)/gb = AB016248/ | 28 | P | 56.9 | P | 39.7 | A | 44.7 |
| | | gi = 3721883/ug = Mm 13081/len = 1721/STRA = for | | | | | | | |
| 18 | 103635_at | Cluster Incl AI648925 uk32h09 x1 Mus musculus cDNA, 3 end/clone = IMAGE-1970753/clone_end = 3/gb = AI648925/ | 143.8 | P | 18.5 | A | 135.5 | A | 145. 2 |
| | | gi = 4729759/ug = Mm 20320/len = 594/STRA = for | | | | | | | |
| 18 | 104326_at | Cluster Incl AI838951 UI-M-AO0-ace-e-06-0-UI s1 Mus musculus cDNA, 3 end/clone = UI-M-AO0-ace-e-06-0-UI/ | 303.7 | P | 80.3 | A | 314.6 | P | 334.6 |
| | | clone_end = 3/gb = AI838951/gi = 5473164/ug = Mm 22456/len = 451/STRA = for | | | | | | | |
| 18 | 96817_at | Cluster Incl AI850550 UI-M-BG1-aij-d-11-0-UI s1 Mus musculus cDNA, 3 end/clone = UI-M-BG1-aij-d-11-0-UI/ | 206.8 | P | 171 | M | 246.3 | A | 236.4 |
| | | clone_end = 3/gb = AI850550/gi = 5494456/ug = Mm 29333/len = 282/STRA = for | | | | | | | |
| 18 | 100906_at | Cluster Incl M68903 Integrin beta 7/cds = (39,2456)/gb = M68903/gi = 349598/ug = Mm 58/len = 2636/STRA = rev | 461.7 | P | 348.3 | M | 443.1 | P | 393.6 |
| 18 | 103756_at | Cluster Incl AA832886 uc91h02 r1 Mus musculus cDNA, 5 end/clone = IMAGE-1433043/clone_end = 5/gb = AA832886/ | 178.5 | P | 102.3 | A | 154.1 | A | 218.2 |
| | | gi = 2906614/ug = Mm.24569/len = 395/STRA = rev | | | | | | | |
| 18 | 93391_at | Cluster Incl AF032995: Crystallin, gamma S/cds = (51,587)/gb = AF032995/gi = 2642336/ug = Mm 6253/len = 702/STRA = for | 65.8 | A | 68.6 | P | 73.4 | P | 48.8 |
| 18 | 93902_at | Cluster Incl AJ250669: Mus musculus gab1 gene for GRB2-associated binding protein 1/cds = (23,2110)/gb = AJ250669/ | 175.6 | P | 124.4 | A | 193.2 | P | 132.7 |
| | | gi = 6165414/ug = Mm.89347/len = 2620/STRA = for | | | | | | | |
| 18 | 94111_r_at | Cluster Incl D10627: Mus musculus mRNA for zinc finger protein, partial cds/cds = (0,1040)/gb = D10627/gi = 220636/ | 140.2 | P | 78.1 | A | 49.6 | A | 65 |
| | | ug = Mm 10509/len = 1041/STRA = for | | | | | | | |
| 18 | 96502_at | Cluster Incl U75646 Phosphate regulating neutral endopeptidases on the X Chromosome/cds = (111,2360)/gb = U75646/ | 106.8 | A | 92.8 | M | 99.3 | P | 111.3 |
| | | gi = 1843530/ug = Mm.2529/len = 2636/STRA = for | | | | | | | |
| 18 | 97107_at | Cluster Incl AA162144 mr19b05 r1 Mus musculus cDNA, 5 end/clone = IMAGE-597873/clone_end = 5/gb = AA162144/ | 46.3 | A | 34 | A | 47.7 | A | 66.3 |
| | | gi = 1737885/ug = Mm.3261/len = 300/STRA = rev | | | | | | | |
| 18 | 99894_at | Cluster Incl AF006201: Mus musculus prostaglandin F2 alpha receptor regulatory protein mRNA, partial cds/cds = (0,198)/ | 161.2 | P | 200.8 | P | 153.3 | P | 153.4 |
| | | gb = AF006201/gi = 2653775/ug = Mm 7999/len = 271/STRA = for | | | | | | | |
| 18 | 100701_r_at | Cluster Incl AB000490 Mouse mRNA for ELP3, complete cds/cds = (245,1636)/gb = AB000490/gi = 1805352/ug = Mm 1416/ | 142.1 | M | 125.9 | A | 168.5 | A | 101.6 |
| | | len = 2981/STRA = for | | | | | | | |
| 18 | 101705_at | Cluster Incl X12809: Mouse period repetitive mRNA (lambda Sp34)/cds = (30,212)/gb = X12809/gi = 53648/ug = Mm 41987/ | 270.4 | A | 261.4 | P | 177.8 | P | 229.4 |
| | | len = 522/STRA = for | | | | | | | |
| 18 | 93778_at | Cluster Incl AA717815 vt98f07 r1 Mus musculus cDNA, 5 end/clone = IMAGE-1179205/clone_end = 5/gb = AA717815/ | 604.8 | M | 505.4 | A | 527.5 | A | 619.6 |
| | | gi = 2730089/ug = Mm 21239/len = 424/STRA = for | | | | | | | |
| 18 | 104608_at | Cluster Incl AI854293: UI-M-BH0-ajs-e-11-0-UI s1 Mus musculus cDNA, 3 end/clone = UI-M-BH0-ajs-e-11-0-UI/ | 54.1 | A | 93 | P | 102.2 | P | 65.7 |
| | | clone_end = 3/gb = AI854293/gi = 5498199/ug = Mm 24143/len = 511/STRA = for | | | | | | | |
| 18 | 94349_at | Cluster Incl AA794923: vr48g02.s1 Mus musculus cDNA, 5 end/clone = IMAGE-1123922/clone_end = 5/gb = AA794923/ | 129.5 | P | 102.3 | A | 100.6 | P | 104.5 |
| | | gi = 2857878/ug = Mm 25189/len = 537/STRA = for | | | | | | | |
| 18 | 97980_at | Cluster Incl L38423: Lymphotoxin B receptor/cds = (185,1432)/gb = L38423/gi = 600222/ug = Mm.3122/len = 1614/STRA = for | 271 | A | 330.4 | A | 314.8 | A | 320.9 |
| 18 | 101857_at | Cluster Incl AB006036 Serine/arginine-rich protein specific kinase 2/cds = (0,2045)/gb = AB006036/gi = 2723281/ | 112.4 | P | 66.3 | A | 89.1 | A | 80.4 |
| | | ug = Mm B709/len = 2300/STRA = for | | | | | | | |
| 18 | 103473_at | Cluster Incl AA623401 vn32c09.r1 Mus musculus cDNA, 5 end/clone = IMAGE-1022896/clone_end = 5/gb = AA623401/ | 365.7 | P | 49.5 | A | 247.4 | A | 471.7 |
| | | gi = 2527277/ug = Mm 21876/len = 345/STRA = for | | | | | | | |
| 18 | 104413_at | Cluster Incl AI834976 UI-M-AM1-afw-d-12-0-UI s1 Mus musculus cDNA, 3 end/clone = UI-M-AM1-afw-d-12-0-UI/ | 60.4 | A | 65.5 | A | 116.9 | P | 101.4 |
| | | clone_end = 3/gb = AI834976/gi = 5469189/ug = Mm.28885/len = 434/STRA = for | | | | | | | |
| 18 | 93899_at | Cluster Incl AJ000654 Mouse p70/p85 S6 kinase gene, 5 flanking region and the first exon/cds = (63,203)/gb = AJ000654/ | 190.3 | P | 98.4 | A | 159.8 | A | 96.4 |
| | | gi = 2330619/ug = Mm 89315/len = 204/STRA = for | | | | | | | |
| 18 | 100326_f_at | Cluster Incl U12889 Killer cell lectin-like receptor, subfamily A, member B/cds = (196,996)/gb = U12889/gi = 602407/ | 56.6 | M | 54.9 | A | 28.3 | A | 63.4 |
| | | ug = Mm.87782/len = 1229/STRA = for | | | | | | | |
| 18 | 96961_at | Cluster Incl AI503821: vm20c01.x1 Mus musculus cDNA, 3 end/clone = IMAGE-990720/clone_end = 3/gb = AI503821/ | 56.4 | P | 104.4 | A | 57.7 | M | 114.2 |
| | | gi = 4401672/ug = Mm 69094/len = 470/STRA = rev | | | | | | | |
| 18 | 102131_f_at | Cluster Incl AU014874 AU014874 Mus musculus cDNA, 3 end/clone = J0703H02/clone_end = 3/gb = AU014874/ | 490.9 | P | 376.9 | A | 472.8 | P | 515 |
| | | gi = 3369665/ug = Mm 43527/len = 294/STRA = rev | | | | | | | |
| 19 | 92616_at | Cluster Incl D10576 Ubiquitin-activating enzyme E1, Chr X/cds = (91,3267)/gb = D10576/gi = 220628/ug = Mm 1104/ | 1508.4 | P | 1414.8 | P | 1542.8 | P | 1658.8 |
| | | len = 3458/STRA = for | | | | | | | |
| 19 | 92793_at | Cluster Incl X57796 Tumor necrosis factor receptor superfamily, member 1a/cds = (272,1636)/gb = X57796/gi = 54848/ | 356.4 | P | 600 | P | 827.9 | P | 979.3 |
| | | ug = Mm 1258/len = 2179/STRA = for | | | | | | | |
| 19 | 93047_at | Cluster Incl AI849144 UI-M-AJ1-agy-c-07-0-UI.s1 Mus musculus cDNA, 3 end/clone = UI-M-AJ1-agy-c-07-0-UI/ | 617.2 | P | 815.7 | P | 847.9 | P | 972.7 |
| | | clone_end = 3/gb = AI849144/gi = 5493050/ug = Mm.1521/len = 439/STRA = for | | | | | | | |
| 19 | 93083_at | Cluster Incl D63423: Mouse mRNA for annexin V, complete cds/cds = (81,1040)/gb = D63423/gi = 939830/ug = Mm 1620/ | 862.5 | P | 1662.5 | P | 1719.1 | P | 1743.2 |
| | | len = 1415/STRA = for | | | | | | | |
| 19 | 93255_at | Cluster lncl X80937 Ral-interacting protein 1/cds = (202,2148)/gb = X80937/gi = 984748/ug = Mm 17009/len = 3661/ | 1351.8 | P | 1361.2 | P | 1595.6 | P | 1512.9 |
| | | STRA = rev | | | | | | | |
| 19 | 93268_at | Cluster Incl AI852001 UI-M-BH0-aiq-f-01-0-UI s1 Mus musculus cDNA, 3 end/clone = UI-M-BH0-aiq-f-01-0-UI/ | 456.5 | P | 789.5 | P | 1007.7 | P | 904.7 |
| | | clone_end = 3/gb = AI852001/gi = 5495907/ug = Mm 17362/len = 431/STRA = for | | | | | | | |
| 19 | 93269_at | Cluster Incl AI848952 UI-M-AJ1-ahd-a-06-0-UI s1 Mus musculus cDNA, 3 end/clone = UI-M-AJ1-ahd-a-06-0-UI/ | 776 | P | 1159.3 | P | 1681.5 | P | 1593.8 |
| | | clone_end = 3/gb = AI848952/gi = 5492858/ug = Mm 17362/len = 482/STRA = for | | | | | | | |
| 19 | 93315_at | Cluster Incl AI852636 UI-M-BH0-aiu-g-03-0-Ul.s1 Mus musculus cDNA, 3 end/clone = UI-M-BH0-aiu-g-03-0-UI/ | 587.7 | P | 627.8 | P | 728 | P | 931.1 |
| | | clone_end = 3/gb = AI852636/gi = 5496542/ug = Mm 18494/len = 365/STRA = for | | | | | | | |
| 19 | 93323_at | Cluster Incl AB031292 Mus musculus Plp2 mRNA for proteolipid protein 2, complete cds/cds = (58,516)/gb = AB031292/ | 3670.8 | P | 3703.6 | P | 3741.3 | P | 4092.9 |
| | | gi = 5771450/ug = Mm 18585/len = 603/STRA = for | | | | | | | |
| 19 | 93336_at | Cluster Incl AW121539: UI-M-BH2 2-aop-e-01-0-UI s1 Mus musculus cDNA, 3 end/clone = UI-M-BH2 2-aop-e-01-0-UI/ | 1336.9 | P | 1435.5 | P | 1419.7 | P | 1499.1 |
| | | clone_end = 3/gb = AW121539/gi = 6096872/ug = Mm 18688/len = 514/STRA = for | | | | | | | |
| 19 | 93753_at | Cluster Incl AI852632: UI-M-BH0-aiu-f-10-0-UI.s1 Mus musculus cDNA, 3 end/clone = UI-M-BH0-aiu-f-10-0-UI/ | 496.3 | P | 602 | P | 886.5 | P | 848.2 |
| | | clone_end = 3/gb = AI852632/gi = 5496538/ug = Mm 21119/len = 346/STRA = for | | | | | | | |
| 19 | 93809_at | Cluster Incl U41736 Ancient ubiquitous protein/cds = (60,1292)/gb = U41736/gi = 1517821/ug = Mm.2146/len = 1499/ | 880.2 | P | 884.9 | P | 975.3 | P | 953.8 |
| | | STRA = for | | | | | | | |
| 19 | 93843_at | Cluster Incl AI845580 UI-M-AQ1-adx-c-01-0-UI s1 Mus musculus cDNA, 3 end/clone = UI-M-AQ1-adx-c-01-0-UI/ | 718.3 | P | 941 | P | 948.8 | P | 934.2 |
| | | clone_end = 3/gb = AI845580/gi = 5489486/ug = Mm.21623/len = 245/STRA = for | | | | | | | |
| 19 | 94297_at | Cluster Incl U16959 FK506 binding protein 5, 51 kDa/cds = (219,1589)/gb = U16959/gi = 915279/ug = Mm 22595/len = 2285/ | 145.1 | P | 466.6 | P | 718.7 | P | 499.4 |
| | | STRA = for | | | | | | | |
| 19 | 94805_f_at | Cluster Incl M33988 Mouse histone H2A 1 gene, complete cds/cds = (45,437)/gb = M33988/gi = 193730/ug = Mm 22676/ | 2979 | P | 2794.5 | P | 3618.9 | P | 3438 |
| | | len = 506/STRA = for | | | | | | | |
| 19 | 94813_at | Cluster Incl X65128 Growth arrest specific 1/cds = (424,1578)/gb = X65128/gi = 51045/ug = Mm 22701/len = 2961/STRA = for | 944.7 | P | 1283.6 | P | 1398.1 | P | 1644.2 |
| 19 | 94850_at | Cluster Incl AJ238894 Mus musculus mRNA for mitochondrial acyl-CoA thioesterase, clone 2/cds = (97,1416)/ | 594 | P | 594.1 | P | 728.7 | P | 710.9 |
| | | gb = AJ238894/gi = 5102775/ug = Mm 23368/len = 1557/STRA = for | | | | | | | |
| 19 | 94868_at | Cluster Incl AW049812 UI-M-BH1-anm-h-04-0-UI s1 Mus musculus cDNA, 3 end/clone = UI-M-BH1-anm-h-04-0-UI/ | 1059.5 | P | 1441.1 | P | 1288.8 | P | 1347.5 |
| | | clone_end = 3/gb = AW049812/gi = 5910341/ug = Mm 23750/len = 571/STRA = for | | | | | | | |
| 19 | 94881_at | Cluster Incl AW048937 UI-M-BH1-amo-d-08-0-UI s1 Mus musculus cDNA, 3 end/clone = UI-M-BH1-amo-d-08-0-UI/ | 1270.2 | P | 1279.1 | P | 1432.2 | P | 1363.2 |
| | | clone_end = 3/gb = AW048937/gi = 5909466/ug = Mm 2387/len = 355/STRA = for | | | | | | | |
| 19 | 95446_at | Cluster Incl AW120502 UI-M-BH1-ami-f-05-0-UI s2 Mus musculus cDNA, 3 end/clone = UI-M-BH1-ami-f-05-0-UI/ | 864.9 | P | 1021.6 | P | 1152 | P | 1052.6 |
| | | clone_end = 3/gb = AW120502/gi = 6095835/ug = Mm 24600/len = 199/STRA = for | | | | | | | |
| 19 | 95520_at | Cluster Incl AI843845 UI-M-AK1-aeu-a-10-0-UI s1 Mus musculus cDNA, 3 end/clone = UI-M-AK1-aeu-a-10-0-UI/ | 181.7 | P | 245.8 | P | 285.6 | P | 298.3 |
| | | clone_end = 3/gb = AI843845/gi = 5478058/ug = Mm 25320/len = 404/STRA = for | | | | | | | |
| 19 | 96056_at | Cluster Incl X80638 M. musculus rhoC mRNA/cds = (40,621)/gb = X80638/gi = 1279555/ug = Mm.262/len = 1036/STRA = for | 579.9 | P | 630.8 | P | 849.7 | P | 888.6 |
| 19 | 96060_at | Cluster Incl U25844 Serine protease inhibitor 3/cds = (67,1203)/gb = U25844/gi = 818902/ug = Mm 2623/len = 1317/ | 843 | P | 1206.8 | P | 1167.4 | P | 1557.4 |
| | | STRA = for | | | | | | | |
| 19 | 96102_i_at | Cluster Incl X92411 RAD23b homolog (S cerevisiae)/cds = (0,1250)/gb = X92411/gi = 1044898/ug = Mm 26879/len = 1251/ | 428.7 | P | 501.4 | P | 679.3 | P | 526.3 |
| | | STRA = for | | | | | | | |
| 19 | 96113_at | Cluster Incl AI846519 UI-M-AN1-aff-e-06-0-UI s1 Mus musculus cDNA, 3 end/clone = UI-M-AN1-aff-e-06-0-UI/ | 601 | P | 838.6 | P | 1141.5 | P | 921.4 |
| | | clone_end = 3/gb = AI846519/gi = 5490425/ug = Mm 26956/len = 234/STRA = for | | | | | | | |
| 19 | 96117_r_at | Cluster Incl AI843732: UI-M-AK1-aer-g-09-0-UI s1 Mus musculus cDNA, 3 end/clone = UI-M-AK1-aer-g-09-0-UI/ | 433 | P | 707.8 | P | 663.4 | P | 623.5 |
| | | clone_end = 3/gb = AI843732/gi = 5477945/ug = Mm 26991/len = 369/STRA = for | | | | | | | |
| 19 | 96709_at | Cluster Incl AI839839: UI-M-AP0-abf-a-09-0-UI s2 Mus musculus cDNA, 3 end/clone = UI-M-AP0-abf-a-09-0-UI/ | 660.2 | P | 861.7 | P | 823.3 | P | 976.3 |
| | | clone_end = 3/gb = AI839839/gi = 5474052/ug = Mm 27608/len = 281/STRA = for | | | | | | | |
| 19 | 97885_at | Clusler Incl AB031386 Mus musculus mRNA for Clast1, complete cds/cds = (196,987)/gb = AB031386/gi = 5804793/ | 1015.5 | P | 1027.7 | P | 1620.2 | P | 1316.8 |
| | | ug = Mm 28385/len = 1207/STRA = for | | | | | | | |
| 19 | 98884_r_at | Cluster Incl AI837311: UI-M-AK0-adf-b-03-0-UI.s1 Mus musculus cDNA, 3 end/clone = UI-M-AK0-adf-b-03-0-UI/ | 761.8 | P | 750.8 | P | 901.5 | P | 983.7 |
| | | clone_end = 3/gb = AI837311/gi = 5471524/ug = Mm.28527/len = 420/STRA = for | | | | | | | |
| 19 | 94465_g_at | Cluster Incl AF029347: Mus musculus chloride channel protein 3 (CLCN3) mRNA, complete cds/cds = (0,2456)/ | 174.9 | P | 159.7 | P | 194.8 | P | 187.6 |
| | | gb = AF029347/gi = 2599549/ug = Mm.28842/len = 2457/STRA = for | | | | | | | |
| 19 | 94473_at | Cluster Incl AW047951: UI-M-BH1-aln-f-04-0-UI s1 Mus musculus cDNA, 3 end/clone = UI-M-BH1-aln-f-04-0-UI/ | 516.7 | P | 646.6 | P | 802.8 | P | 776.6 |
| | | clone_end = 3/gb = AW047951/gi = 5908480/ug = Mm 28853/len = 408/STRA = for | | | | | | | |
| 19 | 94484_at | Cluster Incl AW227778: up14b01.y1 Mus musculus cDNA, 5 end/clone = IMAGE-2654281/clone_end = 5/gb = AW227778/ | 380 | P | 590 | P | 622.4 | P | 626.3 |
| | | gi = 6557074/ug = Mm 28882/len = 460/STRA = for | | | | | | | |
| 19 | 94485_at | Cluster Incl AI840013 UI-M-AH0-acu-e-04-0-UI s1 Mus musculus cDNA, 3 end/clone = UI-M-AH0-acu-e-04-0-UI/ | 860.6 | P | 931.3 | P | 1075.8 | P | 1006.7 |
| | | clone_end = 3/gb = AI840013/gi = 5474226/ug = Mm 28883/len = 356/STRA = for | | | | | | | |
| 19 | 94530_at | Cluster Incl AI840376 UI-M-AH0-acv-d-02-0-UI s1 Mus musculus cDNA, 3 end/clone = UI-M-AH0-acv-d-02-0-UI/ | 181.6 | P | 267.4 | P | 273.2 | P | 356.4 |
| | | clone_end = 3/gb = AI840376/gi = 5474589/ug = Mm 29041/len = 448/STRA = for | | | | | | | |
| 19 | 95082_at | Cluster Incl AI842277 UI-M-AM1-afz-e-02-0-UI.s1 Mus musculus cDNA, 3 end/clone = UI-M-AM1-afz-e-02-0-UI/ | 228.7 | P | 682.8 | P | 972.8 | P | 892.8 |
| | | clone_end = 3/gb = AI842277/gi = 5476530/ug = Mm.29254/len = 475/STRA = for | | | | | | | |
| 19 | 95129_at | Cluster Incl AW121057 UI-M-BH2 3-aof-d-08-0-UI s1 Mus musculus cDNA, 3 end/clone = UI-M-BH2 3-aof-d-08-0-UI/ | 647.3 | P | 680 | P | 1127.3 | P | 941.2 |
| | | clone_end = 3/gb = AW121057/gi = 6096390/ug = Mm 29412/len = 220/STRA = rev | | | | | | | |
| 19 | 95133_at | Cluster Incl U38940 Mus musculus asparagine synthetase mRNA, complete cds/cds = (98,1783)/gb = U38940/gi = 1147604/ | 969.8 | P | 1255 | P | 1165.6 | P | 1631 |
| | | ug = Mm.2942/len = 1928/STRA = for | | | | | | | |
| 19 | 95721_at | Cluster Incl AW120722 UI-M-BH2.3-any-a-09-0-UI s1 Mus musculus cDNA, 3 end/clone = UI-M-BH2 3-any-a-09-0-UI/ | 1005.2 | P | 1173.3 | P | 1520.6 | P | 1325.7 |
| | | clone_end = 3/gb = AW120722/gi = 6096055/ug = Mm 29725/len = 406/STRA = for | | | | | | | |
| 19 | 96278_at | Cluster Incl AI846553 UI-M-AN1-aff-h-09-0-UI s1 Mus musculus cDNA, 3 end/clone = UI-M-AN1-aff-h-09-0-UI/ | 238.7 | P | 284 | P | 360.4 | P | 372 |
| | | clone_end = 3/gb = AI846553/gi = 5490459/ug = Mm 29865/len = 300/STRA = for | | | | | | | |
| 19 | 96359_at | Cluster Incl AI836554 UI-M-AP0-abe-e-12-0-UI s1 Mus musculus cDNA, 3 end/clone = UI-M-AP0-abe-e-12-0-UI/ | 1189 | P | 1407.6 | P | 1395.4 | P | 1479.8 |
| | | clone_end = 3/gb = AI836554/gi = 5470767/ug = Mm 30012/len = 514/STRA = for | | | | | | | |
| 19 | 96919_at | Cluster Incl M64298 ATPase-like vacuolar proton channel/cds = (147,614)/gb = M64298/gi = 199901/ug = Mm.30155/ | 2824.5 | P | 3049.2 | P | 3139.9 | P | 3376.6 |
| | | len = 1153/STRA = for | | | | | | | |
| 19 | 96935_at | Cluster Incl AW011791 ul91f11 x1 Mus musculus cDNA, 3 end/clone = IMAGE-2159469/clone_end = 3/gb = AW011791/ | 643.2 | P | 826 | P | 805.1 | P | 1007.7 |
| | | gi = 5860569/ug = Mm 30181/len = 290/STRA = for | | | | | | | |
| 19 | 96953_at | Cluster Incl AW120786 UI-M-BH2 3-any-g-10-0-UI s1 Mus musculus cDNA, 3 end/clone = UI-M-BH2 3-any-g-10-0-UI/ | 663.5 | P | 701.5 | P | 911.7 | P | 873.3 |
| | | clone_end = 3/gb = AW120786/gi = 6096119/ug = Mm 30211/len = 401/STRA = for | | | | | | | |
| 19 | 97487_at | Cluster Incl X70296 Serine protease Inhibitor 4/cds = (148,1341)/gb = X70296/gi = 551064/ug = Mm 3093/len = 2024/ | 160.9 | P | 163.5 | P | 245 | P | 213 |
| | | STRA = for | | | | | | | |
| 19 | 97498_at | Cluster Incl U41739: Mus musculus RBP associated molecule RAM14-1 mRNA, complete cds/cds = (82,924)/gb = U41739/ | 1950.5 | P | 2692.4 | P | 3047 | P | 3249 |
| | | gi = 2894671/ug = Mm.3126/len = 2270/STRA = for | | | | | | | |
| 19 | 97500_g_at | Cluster Incl AI839950 UI-M-AH0-acs-a-10-0-UI s1 Mus musculus cDNA, 3 end/clone = UI-M-AH0-acs-a-10-0-UI/ | 1557.2 | P | 1592.1 | P | 2319.5 | P | 2481.2 |
| | | clone_end = 3/gb = AI839950/gi = 5474163/ug = Mm 3126/len = 361/STRA = for | | | | | | | |
| 19 | 97551_at | Cluster Incl AI846182 UI-M-AM1-agb-c-09-0-UI.s1 Mus musculus cDNA, 3 end/clone = UI-M-AM1-agb-c-09-0-UI/ | 354.2 | P | 461.4 | P | 537.4 | P | 559 |
| | | clone_end = 3/gb = AI846182/gi = 5490088/ug = Mm 33797/len = 312/STRA = for | | | | | | | |
| 19 | 98051_at | Cluster Incl AI852808 UI-M-BH0-aly-g-09-0-UI.s1 Mus musculus cDNA, 3 end/clone = UI-M-BH0-aly-g-09-0-UI/ | 548.4 | P | 622.3 | P | 816.2 | P | 612.6 |
| | | clone_end = 3/gb = AI852808/gi = 5496714/ug = Mm 34315/len = 359/STRA = for | | | | | | | |
| 19 | 98057_at | Cluster Incl AW121162 UI-M-BH2 3-aoh-g-10-0-UI s1 Mus musculus cDNA, 3 end/clone = UI-M-BH2 3-aoh-g-10-0-UI/ | 80.7 | P | 108.1 | P | 140.5 | P | 155.5 |
| | | clone_end = 3/gb = AW121162/gi = 6096495/ug = Mm 34356/len = 429/STRA = for | | | | | | | |
| 19 | 98081_at | Cluster Incl AI853173: UI-M-BH0-ajh-b-07-0-UI s1 Mus musculus cDNA, 3 end/clone = UI-M-BH0-ajh-b-07-0-UI/ | 286 | P | 279.1 | P | 387.3 | P | 443.9 |
| | | clone_end = 3/gb = AI853173/gi = 5497079/ug = Mm 34570/len = 366/STRA = for | | | | | | | |
| 19 | 98560_at | Cluster Incl AI849152: UI-M-AJ1-agy-d-04-0-UI s1 Mus musculus cDNA, 3 end/clone = UI-M-AJ1-agy-d-04-0-UI/ | 496 | P | 528.6 | P | 602 | P | 749.3 |
| | | clone_end = 3/gb = AI849152/gi = 5493058/ug = Mm 36879/len = 435/STRA = for | | | | | | | |
| 19 | 98580_at | Cluster Incl D28117 Mouse mRNA for magnesium dependent protein phosphatase (protein phosphatase 2C) alpha, | 520.8 | P | 641.4 | P | 783.3 | P | 712.5 |
| | | complete cds/cds = (117,1265)/gb = D28117/gi = 532678/ug = Mm 3766/len = 1387/STRA = for | | | | | | | |
| 19 | 99099_at | Cluster Incl U08378 Mus musculus acute phase response factor (APRF) mRNA, complete cds/cds = (146,2455)/ | 637 | P | 952.2 | P | 886.2 | P | 1317.9 |
| | | gb = U08378/gi = 473889/ug = Mm 3948/len = 2924/STRA = for | | | | | | | |
| 19 | 99606_at | Cluster Incl AB005132: LIM motif-containing protein kinase 2/cds = (355,2208)/gb = AB005132/gi = 2804552/ug = Mm 42927/ | 363.7 | P | 460.4 | P | 480.7 | P | 901 |
| | | len = 3688/STRA = for | | | | | | | |
| 19 | 99622_at | Cluster Incl U20344 Gut enriched Kruppel-like factor/cds = (310,1761)/gb = U20344/gi = 912487/ug = Mm 4325/len = 2756/ | 2376 | P | 2273.3 | P | 3345.2 | P | 2526.8 |
| | | STRA = for | | | | | | | |
| 19 | 99635_at | Cluster Incl AI845183 UI-M-BG0-aht-a-12-0-UI s1 Mus musculus cDNA, 3 end/clone = UI-M-BG0-aht-a-12-0-UI/ | 583.4 | P | 588.3 | P | 792.4 | P | 915.8 |
| | | clone_end = 3/gb = AI845183/gi = 5489089/ug = Mm 43482/len = 412/STRA = for | | | | | | | |
| 19 | 99636_at | Cluster Incl AI848033: UI-M-AH1-agp-f-05-0-UI s1 Mus musculus cDNA, 3 end/clone = UI-M-AH1-agp-f-05-0-UI/ | 397.1 | P | 553.5 | P | 483.9 | P | 526.8 |
| | | clone_end = 3/gb = AI848033/gi = 5491939/ug = Mm 43487/len = 257/STRA = for | | | | | | | |
| 19 | 99638_at | Cluster Incl D17546 Procoliagen, type XV/cds = (0,3422)/gb = D17546/gi = 2160436/ug = Mm 4352/len = 4437/STRA = for | 505.9 | P | 711.7 | P | 783.8 | P | 714.4 |
| 19 | 100095_at | Cluster Incl U37799 Scavenger receptor class B1/cds = (50,1579)/gb = U37799/gi = 1167551/ug = Mm 4603/len = 1785/ | 676.3 | P | 870 | P | 871.3 | P | 871.7 |
| | | STRA = for | | | | | | | |
| 19 | 100134_at | Cluster Incl X77952 Endoglin/cds = (363,2324)/gb = X77952/gi = 458473/ug = Mm 4851/len = 2902/STRA = for | 371.4 | P | 418.9 | P | 522.3 | P | 468.1 |
| 19 | 100530_at | Cluster Incl L07924 Mus musculus guanine nucleotide dissociation stimulator for a ras-related GTPase mRNA, complete | 817.7 | P | 799.3 | P | 1026.3 | P | 915.6 |
| | | cds/cds = (212,2770)/gb = L07924/gi = 193572/ug = Mm 5236/len = 3573/STRA = for | | | | | | | |
| 19 | 100573_f_at | Cluster Incl M14220 Mouse neuroleukin mRNA, complete cds/cds = (52,1728)/gb = M14220/gi = 200064/ug = Mm 589/ | 357.2 | P | 536.4 | P | 514.7 | P | 605.4 |
| | | len = 1985/STRA = for | | | | | | | |
| 19 | 100593_at | Cluster Incl L47600 Mus musculus cardiac troponin T isoform A3b mRNA, complete cds/cds = (56,922)/gb = L47600/ | 99.8 | P | 167.2 | P | 171 | P | 158.5 |
| | | gi = 1161079/ug = Mm 632/len = 1111/STRA = for | | | | | | | |
| 19 | 100599_at | Cluster Incl M94087: Activating transcription factor 4/cds = (270,1310)/gb = M94087/gi = 293723/ug = Mm 641/len = 1391/ | 907.3 | P | 1088.3 | P | 1354.6 | P | 1293.1 |
| | | STRA = for | | | | | | | |
| 19 | 100636_at | Cluster Incl U28656: Eukaryotic translation Initiation factor 4E binding protein 1/cds = (50,403)/gb = U28656/gi = 881557/ | 424.5 | P | 466.9 | P | 606.5 | P | 589.3 |
| | | ug = Mm.6700/len = 793/STRA = for | | | | | | | |
| 19 | 101011_at | Cluster Incl L25913: Chaperonin subunit 4 (delta)/cds = (0,1619)/gb = L25913/gi = 460316/ug = Mm.6821/len = 1847/ | 1509.4 | P | 2076.5 | P | 1959.5 | P | 2090.9 |
| | | STRA = for | | | | | | | |
| 19 | 101089_at | Cluster Incl AF002283 Mus musculus alpha-actinin-2 associated LIM protein mRNA, alternatively spliced product, complete | 424 | P | 600.8 | P | 695.4 | P | 694 |
| | | cds/cds = (119,1069)/gb = AF002283/gi = 3138925/ug = Mm 7342/len = 1485/STRA = for | | | | | | | |
| 19 | 101591_at | Cluster Incl AI852589 UI-M-BH0-alu-b-07-0-UI s1 Mus musculus cDNA, 3 end/clone = UI-M-BH0-alu-b-07-0-UI/ | 385.4 | P | 496.7 | P | 524.7 | P | 444.6 |
| | | clone_end = 3/gb = AI852589/gi = 5496495/ug = Mm.91334/len = 235/STRA = for | | | | | | | |
| 19 | 101975_at | Cluster Incl Z12171 DIk1-like homolog (Drosophila)/cds = (0,1157)/gb = Z12171/gi = 50716/ug = Mm 95633/len = 1158/ | 2951.8 | P | 4047.9 | P | 4151.3 | P | 4371 |
| | | STRA = for | | | | | | | |
| 19 | 102072_g_at | Cluster Incl AI851565 UI-M-BH0-aki-f-01-0-UI.s1 Mus musculus cDNA, 3 end/clone = UI-M-BH0-aki-f-01-0-UI/clone_end = 3/ | 435.3 | P | 602 | P | 597.5 | P | 518.5 |
| | | gb = AI851565/gi = 5495471/ug = Mm 10641/len = 341/STRA = for | | | | | | | |
| 19 | 102332_at | Cluster Incl AI850194 UI-M-BG1-aie-a-01-0-UI s1 Mus musculus cDNA, 3 end/clone = UI-M-BG1-aie-a-01-0-UI/ | 162 | P | 205.5 | P | 292.1 | P | 209.5 |
| | | clone_end = 3/gb = AI850194/gi = 5494100/ug = Mm 10750/len = 294/STRA = rev | | | | | | | |
| 19 | 102389_s_at | Cluster Incl AI841303: UI-M-AM0-adu-h-03-0-UI s1 Mus musculus cDNA, 3 end/clone = UI-M-AM0-adu-h-03-0-UI/ | 176.3 | P | 160 | P | 178.3 | P | 347.4 |
| | | clone_end = 3/gb = AI841303/gi = 5475516/ug = Mm 1222/len = 472/STRA = for | | | | | | | |
| 19 | 102807_at | Cluster Incl AW048054 UI-M-BH1-alv-g-07-0-UI s1 Mus musculus cDNA, 3 end/clone = UI-M-BH1-alv-g-07-0-UI/ | 284.8 | P | 418.1 | P | 445 | P | 325.6 |
| | | clone_end = 3/gb = AW048054/gi = 5908583/ug = Mm 14155/len = 457/STRA = for | | | | | | | |
| 19 | 102815_at | Cluster Incl U65986: Annexin XI/cds = (134,1645)/gb = U65986/gi = 1815638/ug = Mm 1427/len = 2370/STRA = for | 718.7 | P | 1018.1 | P | 1082.2 | P | 1309.6 |
| 19 | 102835_at | Cluster Incl X14972 Mouse mRNA for alpha-adaptin (C)/cds = (150,2966)/gb = X14972/gi = 49879/ug = Mm 14555/len = 3242/ | 415.9 | P | 271.1 | P | 464.9 | P | 464.2 |
| | | STRA = for | | | | | | | |
| 19 | 103002_at | Cluster Incl M27923 Glycoprotein galactosyltransferase beta 1, 4/cds = (216,1376)/gb = M27923/gi = 609527/ug = Mm.15622/ | 1210.3 | P | 1294.4 | P | 1543.7 | P | 1447.7 |
| | | len = 1540/STRA = for | | | | | | | |
| 19 | 103559_at | Cluster Incl M12303 Protein kinase, cAMP dependent, catalytic, alpha/cds = (186,1241)/gb = M12303/gi = 200366/ | 517.5 | P | 792.6 | P | 907 | P | 862.8 |
| | | ug = Mm 19111/len = 2231/STRA = for | | | | | | | |
| 19 | 103584_at | Cluster Incl AW124334 UI-M-BH2.1-apq-f-05-0-UI s1 Mus musculus cDNA, 3 end/clone = UI-M-BH2 1-apq-f-05-0-UI/ | 670.5 | P | 709.1 | P | 1288.9 | P | 902.4 |
| | | clone_end = 3/gb = AW124334/gi = 6099864/ug = Mm 19802/len = 227/STRA = rev | | | | | | | |
| 19 | 104041_at | Cluster Incl AW122255 UI-M-BH2 2-aov-g-06-0-UI s1 Mus musculus cDNA, 3 end/clone = UI-M-BH2.2-aov-g-06-0-UI/ | 670.1 | P | 824.2 | P | 1082.7 | P | 960.2 |
| | | clone_end = 3/gb = AW122255/gi = 6097722/ug = Mm 21458/len = 454/STRA = for | | | | | | | |
| 19 | 104058_at | Cluster Incl AW047528 UI-M-BH1-ama-d-09-0-UI s1 Mus musculus cDNA, 3 end/clone = UI-M-BH1-ama-d-09-0-UI/ | 609 | P | 779.8 | P | 787.9 | P | 725.1 |
| | | clone_end = 3/gb = AW047528/gi = 5908057/ug = Mm 21537/len = 418/STRA = for | | | | | | | |
| 19 | 104083_at | Cluster Incl AI853217 UI-M-BH0-ajh-f-11-0-UI s1 Mus musculus cDNA, 3 end/clone = UI-M-BH0-ajh-f-11-0-UI/ | 236.5 | P | 378.8 | P | 523.5 | P | 497.1 |
| | | clone_end = 3/gb = AI853217/gi = 5497123/ug = Mm 21767/len = 230/STRA = for | | | | | | | |
| 19 | 104110_at | Cluster Incl AW060515: UI-M-BH1-ann-d-07-0-UI s1 Mus musculus cDNA, 3 end/clone = UI-M-BH1-ann-d-07-0-UI/ | 1022.3 | P | 1127.4 | P | 1230.2 | P | 1149.3 |
| | | clone_end = 3/gb = AW060515/gi = 6008266/ug = Mm 21919/len = 330/STRA = for | | | | | | | |
| 19 | 104121_at | Cluster Incl M90365 Junction plakoglobin/cds = (0,1865)/gb = M90365/gi = 1389681/ug = Mm 21990/len = 1878/STRA = rev | 1843.3 | P | 1736.9 | P | 2405.1 | P | 2376.6 |
| 19 | 104139_at | Cluster Incl U16162 Mus musculus prolyl 4-hydroxylase alpha(l)-subunit mRNA, partial cds/cds = (0,1582)/gb = U16162/ | 86.4 | P | 216.6 | P | 206.7 | P | 185.1 |
| | | gi = 836897/ug = Mm 2212/len = 2129/STRA = for | | | | | | | |
| 19 | 104145_at | Cluster Incl U81030 Treacher Collins Franceschetti syndrome 1, homolog/cds = (48,3956)/gb = U81030/gi = 2145148/ | 423.3 | P | 488.5 | P | 517.5 | P | 636.2 |
| | | ug = Mm 2215/len = 4428/STRA = for | | | | | | | |
| 19 | 104554_at | Cluster Incl L25674: Mus musculus ear-2 transcription factor mRNA, complete cds/cds = (113,1282)/gb = L25674/gi = 409757/ | 529.8 | P | 517.6 | P | 576.3 | P | 584.8 |
| | | ug = Mm 2376/len = 1643/STRA = for | | | | | | | |
| 19 | 104714_at | Cluster Incl AW125299 UI-M-BH2 1-apy-c-05-0-UI s1 Mus musculus cDNA, 3 end/clone = UI-M-BH2 1-apy-c-05-0-UI/ | 383.4 | P | 402.2 | P | 559.2 | P | 492.7 |
| | | clone_end = 3/gb = AW125299/gi = 6100829/ug = Mm 24477/len = 374/STRA = for | | | | | | | |
| 19 | 94345_at | Cluster Incl AI843709: UI-M-AK1-aer-e-08-0-UI s1 Mus musculus cDNA, 3 end/clone = UI-M-AK1-aer-e-08-0-UI/ | 277.7 | P | 408.6 | P | 450.8 | P | 521.8 |
| | | clone_end = 3/gb = AI843709/gi = 5477922/ug = Mm 25142/len = 419/STRA = rev | | | | | | | |
| 19 | 94357_at | Cluster Incl AA591002 vn61e07.r1 Mus musculus cDNA, 5 end/clone = IMAGE-1025700/clone_end = 5/gb = AA591002/ | 52.4 | P | 84.1 | P | 82.1 | P | 125 |
| | | gi = 2404315/ug = Mm.25237/len = 504/STRA = for | | | | | | | |
| 19 | 95016_at | Cluster Incl D50086 Mouse mRNA for neuropilin, complete cds/cds = (347,3118)/gb = D50086/gi = 1783300/ug = Mm.27448/ | 565.3 | P | 1033.1 | P | 847.9 | P | 1125.8 |
| | | len = 3652/STRA for | | | | | | | |
| 19 | 95023_at | Cluster Incl AW050353 UI-M-BH1-ang-d-12-0-UI.s1 Mus musculus cDNA, 3 end/clone = UI-M-BH1-ang-d-12-0-UI/ | 1223.5 | P | 1472.8 | P | 1683.6 | P | 1673.8 |
| | | clone_end = 3/gb = AW050353/gi = 5910882/ug = Mm 27490/len = 514/STRA = for | | | | | | | |
| 19 | 95602_at | Cluster Incl AI848135 UI-M-AP1-agk-h-05-0-UI s1 Mus musculus cDNA, 3 end/clone = UI-M-AP1-agk-h-05-0-UI/ | 957.1 | P | 1286 | P | 1354.3 | P | 1234.5 |
| | | clone_end = 3/gb = AI848135/gi = 5492041/ug = Mm 27942/len = 365/STRA = for | | | | | | | |
| 19 | 96157_at | Cluster Incl AI839059 UI-M-AK0-adi-c-06-0-UI s1 Mus musculus cDNA, 3 end/clone = UI-M-AK0-adi-c-06-0-UI/ | 841.5 | P | 1087.4 | P | 1118.6 | P | 1283.3 |
| | | clone_end = 3/gb = AI839059/gi = 5473272/ug = Mm 28324/len = 514/STRA = for | | | | | | | |
| 19 | 96748_i_at | Cluster Incl AA619554 vo88e08 r1 Mus musculus cDNA, 5 end/clone = IMAGE-1066214/clone_end = 5/gb = AA619554/ | 153.3 | P | 114.3 | P | 224.9 | P | 167.4 |
| | | gi = 2523430/ug = Mm 28958/len = 468/STRA = for | | | | | | | |
| 19 | 96829_at | Cluster Incl AW047083 UI-M-BH1-alr-f-05-0-UI s1 Mus musculus cDNA, 3 end/clone = UI-M-BH1-alr-f-05-0-UI/ | 663 | P | 749.5 | P | 1001 | P | 938.4 |
| | | clone_end = 3/gb = AW047083/gi = 5907612/ug = Mm 29398/len = 311/STRA = rev | | | | | | | |
| 19 | 97424_at | Cluster Incl AW049647 UI-M-BH1-and-g-07-0-UI s1 Mus musculus cDNA, 3 end/clone = UI-M-BH1-and-g-07-0-UI/ | 488.7 | P | 553.4 | P | 733.9 | P | 604.9 |
| | | clone_end = 3/gb = AW049647/gi = 5910176/ug = Mm 30009/len = 487/STRA = for | | | | | | | |
| 19 | 98031_at | Cluster Incl AF027707 Mus musculus apoptosis activator Mtd (Mtd) mRNA, complete cds/cds = (188,829)/gb = AF027707/ | 620.5 | P | 669.3 | P | 836 | P | 848.5 |
| | | gi = 2689659/ug = Mm 3295/len = 1430/STRA = for | | | | | | | |
| 19 | 99027_at | Cluster Incl L35049 Bcl2-like/cds = (242,943)/gb = L35049/gi = 561740/ug = Mm 3882/len = 979/STRA = for | 423.8 | P | 408.8 | P | 469.2 | P | 676.7 |
| 19 | 99034_at | Cluster Incl Y15001 Mus musculus mRNA for Iroquois homeobox protein 3/cds = (0,1523)/gb = Y15001/gi = 2765437/ | 321.3 | P | 433.3 | P | 599.2 | P | 523.4 |
| | | ug = Mm 39039/len = 1937/STRA = for | | | | | | | |
| 19 | 100492_at | Cluster Incl AW122807: UI-M-BH2.1-aoy-h-10-0-UI s1 Mus musculus cDNA, 3 end/clone = UI-M-BH2 1-aoy-h-10-0-UI/ | 362.5 | P | 611.1 | P | 723.8 | P | 606.8 |
| | | clone_end = 3/gb = AW122807/gi = 6098337/ug = Mm 5063/len = 142/STRA = for | | | | | | | |
| 19 | 100515_at | Cluster Incl X54056 Proprotein convertase subtillsin/kexin type 3/cds = (94,2475)/gb = X54056/gi = 50996/ug = Mm 5241/ | 966.5 | P | 1316.8 | P | 1339.2 | P | 1348 |
| | | len/3978/STRA = rev | | | | | | | |
| 19 | 100986_at | Cluster Incl AF055889: Mus musculus LIM protein FHL2 (Fhl2) mRNA, complete cds/cds = (11,850)/gb = AF055889/ | 588.1 | P | 608.2 | P | 663.5 | P | 702.4 |
| | | gi = 3025852/ug = = Mm.6799/len = 1300/STRA = for | | | | | | | |
| 19 | 101457_at | Cluster Incl L16956: Janus Kinase 2/cds = (93,3482)/gb = L16956/gi = 309463/ug = Mm 809/len = 3629/STRA = for | 66.8 | P | 83.1 | P | 148.4 | P | 200.7 |
| 19 | 101836_at | Cluster Incl D45859 Protein phosphatase 1B, magnesium dependent, beta isoform/cds = (0,461)/gb = D45B59/gi = 961469/ | 34 | P | 41.1 | P | 94.7 | P | 73.9 |
| | | ug = Mm 849/len = 546/STRA = for | | | | | | | |
| 19 | 101876_s_at | Cluster Incl M35247 Histocompatibility 2, T region locus 17/cds = (34,168)/gb = M35247/gi = 199685/ug = Mm 88092/ | 200 | P | 257.3 | P | 418.5 | P | 376.4 |
| | | len = 1497/STRA = for | | | | | | | |
| 19 | 101881_g_at | Cluster Incl L22545 Procollagen, type XVIII, alpha 1/cds = (0,3866)/gb = L22545/gi = 348968/ug = Mm 88304/len = 4031/ | 371.5 | P | 376.4 | P | 539.2 | P | 567.9 |
| | | STRA = rev | | | | | | | |
| 19 | 102201_s_at | Cluster Incl L42177 Presenilin 1/cds = (187,1590)/gb = L42177/gi = 904129/ug = Mm 998/len = 1964/STRA = for | 870.8 | P | 990.8 | P | 1052.1 | P | 1001.8 |
| 19 | 102204_at | Cluster Incl L36435 Kreisler (maf-related) leucine zipper protein/cds = (169,1140)/gb = L35435/gi = 625043/ug = Mm 100216/ | 479 | P | 363.1 | P | 847.2 | P | 705.4 |
| | | len = 1458/STRA = for | | | | | | | |
| 19 | 103527_at | Cluster Incl AW124360 UI-M-BH2 1-apq-h-07-0-UI.s1 Mus musculus cDNA, 3 end/clone = UI-M-BH2.1-apq-h-07-0-UI/ | 68.9 | P | 117.2 | P | 234.7 | P | 109.9 |
| | | clone_end = 3/gb = AW124360/gi = 6099890/ug = Mm 23011/len = 232/STRA = rev | | | | | | | |
| 19 | 103542_at | Cluster Incl AW215059 up06a04 y1 Mus musculus cDNA, 5 end/clone = IMAGE-2651214/clone_end = 5/gb = AW215059/ | 530.6 | P | 630.5 | P | 1027.8 | P | 922.1 |
| | | gi = 6525808/ug = Mm 23806/len = 245/STRA = rev | | | | | | | |
| 19 | 104217_at | Cluster Incl AW045753 UI-M-BH1-akt-a-10-0-UI s1 Mus musculus cDNA, 3 end/clone = UI-M-BH1-akt-a-10-0-UI/ | 356.2 | P | 330.8 | P | 427.1 | P | 331.9 |
| | | clone_end = 3/gb = AW045753/gi = 5906282/ug = Mm.27893/len = 407/STRA = rev | | | | | | | |
| 19 | 104221_at | Cluster Incl AB017189 Mus musculus mRNA for 4F2/CD98 light chain, complete cds/cds = (26,1564)/gb = AB017189/ | 736.7 | P | 790.1 | P | 989 | P | 1007.1 |
| | | gi = 4519255/ug = Mm 27943/len = 3441/STRA = for | | | | | | | |
| 19 | 104241_at | Cluster Incl AI854379: UI-M-BG1-aic-f-11-0-UI s1 Mus musculus cDNA, 3 end/clone = UI-M-BG1-aic-f-11-0-UI/ | 632.6 | P | 774.9 | P | 1303.4 | P | 851.9 |
| | | clone_end = 3/gb = AI854379/gi = 5498285/ug = Mm.28184/len = 374/STRA = rev | | | | | | | |
| 19 | 104514_at | Cluster Incl AF057285: Mus musculus intersectin-EH binding protein Ibp1 mRNA, partial cds/cds = (0,639)/gb = AF057285/ | 1271.3 | P | 2060.8 | P | 2244.4 | P | 2013 |
| | | gi = 3063646/ug = Mm 3091/len = 1048/STRA = for | | | | | | | |
| 19 | 92245_at | Cluster Incl AI642262 vn75a09 y1 Mus musculus cDNA, 5 end/clone = IMAGE-1037752/clone_end = 5/gb = AI642262/ | 221 | P | 385.2 | P | 315.9 | P | 471.4 |
| | | gi = 4720737/ug = Mm 35019/len = 255/STRA = rev | | | | | | | |
| 19 | 93195_at | Cluster Incl AI957146 ul76b03 x1 Mus musculus cDNA, 3 end/clone = IMAGE-2136461/clone_end = 3/gb = AI957146/ | 203.7 | P | 301 | P | 427.5 | P | 305.8 |
| | | gi = 5749855/ug = Mm 5601/len = 465/STRA = for | | | | | | | |
| 19 | 93624_at | Cluster Incl AI225296 ue86b01 y1 Mus musculus cDNA, 5 end/clone = IMAGE-1497961/clone_end = 5/gb = AI225296/ | 143.2 | P | 142.6 | P | 241.3 | P | 286.9 |
| | | gi = 3808357/ug = Mm 7446/len = 418/STRA = for | | | | | | | |
| 19 | 93673_at | Cluster Incl U78109 Neurturin/cds = (348,935)/gb = U78109/gi = 1731678/ug = Mm 8074/len = 1023/STRA = for | 706.3 | P | 758.7 | P | 801.4 | P | 914.3 |
| 19 | 93712_at | Cluster Incl AF095640 Mus musculus cyclin T1 mRNA, complete cds/cds = (0,2174)/gb = AF095640/gi = 4106516/ | 309.9 | P | 329.1 | P | 508.6 | P | 421.4 |
| | | ug = Mm 86538/len = 2175/STRA = for | | | | | | | |
| 19 | 94192_at | Cluster Incl Y17860 Mus musculus mRNA for ganglioside-Induced differentiation associated protein 10/cds = (2531,2917)/ | 207.3 | P | 207.1 | P | 198 | P | 312.9 |
| | | gb = Y17860/gi = 3378471/ug = Mm 12950/len = 5011/STRA = for | | | | | | | |
| 19 | 94794_at | Cluster Incl X52561: Mouse gene for ferritin H subunit/cds = (166,714)/gb = X52561/gi = 50953/ug = Mm 1776/len = 860/ | 3759.4 | P | 4097.4 | P | 4610.8 | P | 5640 |
| | | STRA = for | | | | | | | |
| 19 | 96596_at | Cluster Incl U52073 Mus musculus TDD5 mRNA, complete cds/cds = (135,1280)/gb = U52073/gi = 1903378/ug = Mm 30837/ | 317.3 | P | 405.9 | P | 764 | P | 635.2 |
| | | len = 2916/STRA = for | | | | | | | |
| 19 | 97786_at | Cluster Incl AJ011118 Mus musculus mRNA for a skeletal muscle and cardiac protein/cds = (10,996)/gb = AJ011118/ | 183.8 | P | 149.1 | P | 297 | P | 359.4 |
| | | gi = 5420271/ug = Mm 41992/len = 1100/STRA = for | | | | | | | |
| 19 | 98868_at | Cluster Incl 31532 B-cell leukemia/lymphoma 2/cds = (1197,1796)/gb = L31532/gi = 468337/ug = Mm 5155/len = 1897/ | 61.6 | P | 100.8 | P | 138 | P | 125.8 |
| | | STRA = for | | | | | | | |
| 19 | 99849_at | Cluster Incl C85523 C85523 Mus musculus cDNA, 3 end/clone = J0209F01/clone_end = 3/gb = C85523/gi = 2917480/ | 448.7 | P | 769.8 | P | 4708.5 | P | 3902.2 |
| | | ug = Mm 63525/len = 566/STRA = rev | | | | | | | |
| 19 | 100332_s_at | Cluster Incl AF093853 Mus musculus 1-Cys peroxiredoxin protein 2 gene, complete cds/cds = (0,674)/gb = AF093853/ | 334.2 | P | 426.7 | P | 476.2 | P | 488 |
| | | gi = 3769943/ug = Mm 87841/len = 675/STRA = for | | | | | | | |
| 19 | 95796_g_at | Cluster Incl U96810 Mus musculus Sup14h2 gene, complete cds/cds = (0,353)/gb = U96810/gi = 3779195/ug = Mm 87825/ | 546 | P | 579.3 | P | 704.7 | P | 830.4 |
| | | len = 354/STRA = for | | | | | | | |
| 19 | 96391_at | Cluster Incl C80836 C80836 Mus musculus cDNA, 3 end/clone = J0089A10/clone_end = 3/gb = C80836/gi = 2521166/ | 195.8 | P | 210.1 | P | 243 | P | 240.5 |
| | | ug = Mm.25089/len = 401/STRA = rev | | | | | | | |
| 19 | 97601_f_at | Cluster Incl AW259538: up30a03 x1 Mus musculus cDNA, 3 end/clone = IMAGE-2655820/clone_end = 3/gb = AW259538/ | 2369.3 | P | 2501.7 | P | 2674.9 | P | 2567.6 |
| | | gi = 6632519/ug = Mm 89688/len = 374/STRA = rev | | | | | | | |
| 19 | 93492_at | Cluster Incl AB013469 Mus musculus CLM2 gene for cytohesin 2, complete and partial cds, alternative splicing/ | 237.4 | P | 648.5 | P | 999.8 | P | 982.8 |
| | | cds = (0,1154)/gb = AB013469/gi = 3660542/ug = Mm 19097/len = 1155/STRA = for | | | | | | | |
| 19 | 94299_at | Cluster Incl AI839731: UI-M-AP0-abe-a-03-0-UI s2 Mus musculus cDNA, 3 end/clone = UI-M-AP0-abe-a-03-0-UI/ | 153.1 | P | 229.8 | P | 334.4 | P | 311.2 |
| | | clone_end = 3/gb = AI839731/gi = 5473939/ug = Mm 22597/len = 359/STRA = for | | | | | | | |
| 19 | 97915_at | Cluster Incl AW124144: UI-M-BH2 1-apr-e-12-0-UI s1 Mus musculus cDNA, 3 end/clone = UI-M-BH2 1-apr-e-12-0-UI/ | 394.9 | P | 391 | P | 472.4 | P | 495.4 |
| | | clone_end = 3/gb = AW124144/gi = 6099674/ug = Mm 28491/len = 442/STRA = for | | | | | | | |
| 19 | 99677_at | Cluster Incl AI849559: UI-M-AH1-agr-b-09-0-UI s1 Mus musculus cDNA, 3 end/clone = UI-M-AH1-agr-b-09-0-UI/ | 301.6 | P | 330 | P | 409.1 | P | 443.6 |
| | | clone_end = 3/gb = AI849559/gi = 5493465/ug = Mm 44218/len = 371/STRA = for | | | | | | | |
| 19 | 104562_at | Cluster lncl AW049632: UI-M-BH1-and-f-02-0-UI.s1 Mus musculus cDNA, 3 end/clone = UI-M-BH1-and-f-02-0-UI/ | 228.4 | P | 261.7 | P | 293 | P | 344.4 |
| | | clone_end = 3/gb = AW049632/gi = 5910181/ug = Mm 23838/len = 385/STRA = for | | | | | | | |
| 19 | 96205_at | Cluster Incl AW048272: UI-M-BH1-aly-e-02-0-UI s1 Mus musculus cDNA, 3 end/clone = UI-M-BH1-aly-e-02-0-UI/ | 37.9 | A | 260.9 | P | 399 | P | 343.2 |
| | | clone_end = 3/gb = AW048272/gi = 5908801/ug = Mm.28638/len = 465/STRA = for | | | | | | | |
| 19 | 97319_at | Cluster Incl AF084466 Mus muaculus Ras-like GTP-binding protein Red mRNA, complete cds/cds = (8,932)/gb = AF084466/ | 188.2 | P | 277.3 | P | 259.1 | P | 424.3 |
| | | gi = 3462897/ug = Mm 29467/len = 934/STRA = for | | | | | | | |
| 19 | 97363_at | Cluster Incl M95408 Focal adhesion kinase/cds = (109,3267)/gb = M95408/gi = 193223/ug = Mm 29674/len = 4211/STRA = for | −1.8 | P | 17.7 | P | 98.3 | P | 14.7 |
| 19 | 97398_at | Cluster Incl AI849831: UI-M-AL1-aho-h-05-0-UI s1 Mus musculus cDNA, 3 end/clone = UI-M-AL1-aho-h-05-0-UI/ | 117.9 | P | 129.7 | P | 138.9 | P | 179.1 |
| | | clone_end = 3/gb = AI849831/gi = 5493777/ug = Mm 29856/len = 362/STRA = for | | | | | | | |
| 19 | 97413_at | Cluster Incl AI121305: uc30b06.r1 Mus musculus cDNA, 5 end/clone = IMAGE-1399475/clone_end = 5/gb = AI121305/ | 175.5 | A | 156.4 | P | 474.6 | P | 828.9 |
| | | gi = 3521629/ug = Mm.29959/len = 358/STRA = for | | | | | | | |
| 19 | 98472_at | Cluster Incl Y00629: Histocompatibility 2, T region locus 23/cds = (32,1105)/gb = Y00629/gi = 51013/ug = Mm.35016/ | 316 | P | 2431 | P | 471.3 | P | 406.8 |
| | | len = 1282/STRA = for | | | | | | | |
| 19 | 102627_at | Cluster Incl AF061569 Mus musculus coding region determinant binding protein mRNA, complete cds/cds = (130,1863)/ | 120.9 | A | 118.6 | P | 298.1 | P | 212.7 |
| | | gb = AF061569/gi = 3273748/ug = Mm 12919/len = 2223/STRA = for | | | | | | | |
| 19 | 104201_at | Cluster Incl AA718649 vt86g08 r1 Mus musculus cDNA, 5 end/clone = IMAGE-1178078/clone_end = 5/gb = AA718649/ | 553.8 | A | 537.6 | P | 605.6 | P | 793.6 |
| | | gi = 2730923/ug = Mm 27525/len = 479/STRA = for | | | | | | | |
| 19 | 92189_at | Cluster Incl AI846017: UI-M-AP1-agl-f-08-0-UI s1 Mus musculus cDNA, 3 end/clone = UI-M-AP1-agl-f-08-0-UI/clone_end = 3/ | 133.2 | P | 203.1 | P | 234.4 | P | 224.5 |
| 19 | 99397_at | Cluster Incl U97572 Excision repair 2/cds = (0,2282)/gb = U97572/gi = 2114483/ug = Mm 56990/len = 2283/STRA = for | 277.2 | P | 298.8 | P | 375 | P | 446 |
| 19 | 99915_at | Cluster Incl L41352 Amphiregulin/cds = (0,746)/gb = L41352/gi = 845598/ug = Mm.8039/len = 747/STRA = for | 78.1 | P | 63.2 | P | 91.1 | P | 192.3 |
| 19 | 101676_at | Cluster Incl U13705 Glutathione peroxidase 3/cds = (16,696)/gb = U13705/gi = 536927/ug = Mm 7156/len = 1415/STRA = for | 326.5 | P | 334.5 | P | 532.3 | P | 876.8 |
| 19 | 95095_at | Cluster Incl U90435 Mus musculus flotillin mRNA, complete cds/cds = (159,1445)/gb = U90435/gi = 2149603/ug = Mm.2931/ | −53.1 | P | −48.3 | P | 222 | P | 255.5 |
| | | len = 1702/STRA = for | | | | | | | |
| 19 | 101014_at | Cluster Incl Y09864 M musculus mRNA for type I interferon receptor, IFNaR2b/cds = (220,963)/gb = Y09864/gi = 2467245/ | 77.5 | P | 70.7 | A | 148.3 | P | 137.8 |
| | | ug = Mm 6834/len = 2586/STRA = for | | | | | | | |
| 19 | 103053_at | Cluster Incl X15784 Myogenin/cds = (48,788)/gb = X15784/gi = 53303/ug = Mm 16528/len = 1474/STRA = for | 165.6 | A | 181.1 | P | 242 | P | 309.7 |
| 19 | 103335_at | Cluster Incl U55060 Mus musculus beta-galactoside binding lectin mRNA, complete cds/cds = (34,1002)/gb = U55060/ | 588.7 | P | 454.8 | P | 731.3 | P | 960.3 |
| | | gi = 1916601/ug = Mm 18087/len = 1418/STRA = for | | | | | | | |
| 19 | 103557_at | Cluster Incl AI850046 UI-M-BG0-aia-b-11-0-U1 s1 Mus musculus cDNA, 3 end/clone = UI-M-BG0-aia-b-11-0-UI/ | 346.8 | M | 452.1 | P | 536 | P | 425.5 |
| | | clone_end = 3/gb = AI850046/gi = 5493952/ug = Mm 19091/len = 484/STRA = for | | | | | | | |
| 19 | 104152_at | Cluster Incl AA960466 ub58f11 s1 Mus musculus cDNA, 3 end/clone = IMAGE-1381965/clone_end = 3/gb = AA960466/ | 640.8 | P | 799.3 | P | 1077.8 | P | 1015.1 |
| | | gi = 3126366/ug = Mm 22193/len = 202/STRA = for | | | | | | | |
| 19 | 103737_at | Cluster Incl AB016780 Mus musculus mRNA for Glutamine-fructose-6-phosphate amidotransferase 2, complete cds/ | 211 | P | 437.5 | P | 475.9 | P | 331 |
| | | cds = (48,2096)/gb = AB016780/gi = 4239878/ug = Mm 24402/len = 2958/STRA = for | | | | | | | |
| 19 | 104645_at | Cluster Incl AI853712.UI-M-BH0-ajq-d-05-0-UI s1 Mus musculus cDNA, 3 end/clone = UI-M-BH0-ajq-d-05-0-UI/ | 176.4 | P | 181.9 | A | 339.7 | P | 352.3 |
| | | clone_end = 3/gb = AI853712/gi = 5497583/ug = Mm 31321/len = 496/STRA = rev | | | | | | | |
| 19 | 93865_s_at | Cluster Incl M35244: Histocompatibility 2, T region locus 10/cds = UNKNOWN/gb = M35244/gi = 199063/ug = Mm.87776/ | 169.4 | A | 230.9 | P | 382.9 | P | 419 |
| | | len = 1714/STRA = for | | | | | | | |
| 19 | 95910_f_at | Cluster Incl AI844780 UI-M-AL1-ahq-g-08-0-UI s1 Mus musculus cDNA, 3 end/clone = UI-M-AL1-ahq-g-08-0-UI/ | 241.5 | P | 226.4 | P | 331.8 | P | 314 |
| | | clone_end = 3/gb = AI844780/gi = 5488686/ug = Mm 24440/len = 200/STRA = for | | | | | | | |
| 19 | 93283_at | Cluster Incl AA869401 vq07f04 r1 Mus musculus cDNA, 5 end/clone = IMAGE-1093567/clone_end = 5/gb = AA869401/ | 470.2 | A | 395.7 | P | 554.7 | P | 802.8 |
| | | gi = 2964846/ug = Mm 1786/len = 793/STRA = for | | | | | | | |
| 19 | 96646_at | Cluster Incl AI846253 UI-M-AK1-aez-b-10-0-UI s1 Mus musculus cDNA, 3 end/clone = UI-M-AK1-aez-b-10-0-UI/ | 163.1 | P | 243.1 | P | 230.9 | P | 231.5 |
| | | clone_end = 3/gb = AI846253/gi = 5490159/ug = Mm 27292/len = 311/STRA = for | | | | | | | |
| 19 | 99100_at | Cluster Incl AI837104 UI-M-AK0-adc-d-07-0-UI s1 Mus musculus cDNA, 3 end/clone = UI-M-AK0-adc-d-07-0-UI/ | 194.8 | A | 386 | P | 599.5 | P | 611.3 |
| | | clone_end = 3/gb = AI837104/gi = 5471317/ug = Mm 3948/len = 413/STRA = for | | | | | | | |
| 19 | 101956_at | Cluster Incl AI834849 UI-M-AN0-acm-d-08-0-UI s2 Mus musculus cDNA, 3 end/clone = UI-M-AN0-acm-d-08-0-UI/ | 725.9 | P | 542.3 | A | 776.7 | P | 849.7 |
| | | clone_end= 3/gb = AI834849/gi = 5469062/ug = Mm 9210/len = 176/STRA = for | | | | | | | |
| 19 | 102849_at | Cluster Incl D88159 Potassium inwardly rectifying channel, subfamily J, member 8/cds = (203,1477)/gb = D88159/ | 87.4 | A | 184.6 | A | 187.9 | P | 208.4 |
| | | gi = 1881405/ug = Mm 1482/len = 1712/STRA = for | | | | | | | |
| 19 | 101934_at | Cluster Incl AI851119 UI-M-BH0-ajx-a-08-0-UI s1 Mus musculus cDNA, 3 end/clone = UI-M-BH0-ajx-a-08-0-UI/ | 53.8 | A | 30.9 | A | 117.4 | P | 106.8 |
| | | clone_end = 3/gb = AI851119/gi = 5495065/ug = Mm.946/len = 354/STRA = for | | | | | | | |
| 19 | 94147_at | Cluster Incl M33960: Mouse plasminogen activator inhibitor (PAI-1) mRNA, complete cds/cds = (131,1339)/gb = M33960/ | 161.9 | A | 103.5 | A | 183 | P | 221.6 |
| | | gi = 200219/ug = Mm.1263/len = 2999/STRA = for | | | | | | | |
| 19 | 96418_r_at | Cluster Incl M18399 Mouse homeo box (Hox2.4) region/cds = (0,389)/gb = M18399/gi = 193940/ug = Mm 88400/len = 390/ | 475 | P | 438.2 | P | 627.3 | P | 738.4 |
| | | STRA = for | | | | | | | |
| 19 | 93352_at | Cluster Incl M55154.Mouse transglutaminase (TGase) mRNA, complete cds/cds = (29,2086)/gb = M55154/gi = 201940/ | 36 | A | 92.2 | P | 175.9 | P | 160.9 |
| | | ug = Mm 18843/len = 3483/STRA = for | | | | | | | |
| 19 | 100552_at | Cluster Incl M28233: Mouse interferon-gamma receptor mRNA, complete cds/cds = (101,1534)/gb = M28233/gi = 194131/ | 155.9 | A | 191.8 | P | 267.5 | A | 224.1 |
| | | ug = Mm 549/len = 2093/STRA = for | | | | | | | |
| 19 | 102845_at | Cluster Incl A1836034 UI-M-AQ0-aad-f-06-0-UI s2 Mus musculus cDNA, 3 end/clone = Ul-M-AQ0-aad-f-06-0-UI/ | 130.3 | A | 272.6 | M | 403.4 | P | 438.4 |
| | | clone_end = 3/gb = AI838034/gi = 5470212/ug = Mm 14741/len = 389/STRA = rev | | | | | | | |
| 19 | 102988_at | Cluster Incl U92477: Mus musculus AblSH3 binding protein mRNA, partial cds/cds = (0,1440)/gb = U92477/gi = 1928963/ | 560.4 | A | 812.8 | P | 921.5 | P | 903.3 |
| | | ug = Mm 1531/len = 2188/STRA = for | | | | | | | |
| 19 | 104548_at | Cluster Incl Y15443 Mus musculus mRNA for 50C15 protein/cds = (20,454)/gb = Y15443/gi = 2624230/ug = Mm 23697/ | 143.7 | P | 239.2 | P | 256.6 | P | 259.6 |
| | | len = 721/STRA = for | | | | | | | |
| 19 | 94423_at | Cluster Incl AW049551 UI-M-BH1-anf-f-04-0-UI s1 Mus musculus cDNA, 3 end/clone UI-M-BH1-anf-f-04-0-UI/ | 30 | A | 6.2 | A | 142 | P | 170.2 |
| | | clone_end = 3/gb = AW049551/gi = Mm 26282/len = 447/STRA = for | | | | | | | |
| 19 | 94997_at | Cluster Incl AF060883 Mus musculus endomucin mRNA, complete cds/cds = (97,843)/gb = AF060883/gi = 4159992/ | 160.2 | M | 188.8 | P | 236.4 | P | 247.9 |
| | | ug = Mm.27343/len = 1374/STRA = for | | | | | | | |
| 19 | 96167_at | Cluster Incl AI643420: vv24h01.x1 Mus musculus cDNA, 3 end/clone = IMAGE-1223377/clone_end = 3/gb = AI643420/ | 341 | A | 321 | A | 595.9 | P | 543.1 |
| | | gi = 4721895/ug = Mm 28373/len = 195/STRA = for | | | | | | | |
| 19 | 104268_at | Cluster Incl X51975: Interleukin 6 receptor, alpha/cds = (0,1382)/gb = X51975/gi = 49725/ug = Mm 2856/len = 2499/STRA = for | −102.5 | M | −120.4 | A | 357.2 | P | 592 |
| 19 | 99916_at | Cluster Incl D90242: Protein kinase C, eta/cds = (59,2110)/gb = D90242/gi = 220526/ug = Mm 8040/len = 2176/STRA = for | 48.6 | P | 57.2 | A | 121.4 | P | 108.2 |
| 19 | 94288_at | Cluster Incl J03482 Mouse histone H1 gene, complete cds/cds = (42,680)/gb = J03482/gi = 193847/ug = Mm 22570/len = 725/ | 509.2 | A | 248.1 | A | 105.7 | P | 896.2 |
| | | STRA = for | | | | | | | |
| 19 | 102430_at | Cluster Incl X51397: Myeloid differentiation primary response gene 88/cds = (109,840)/gb = X51397/gi = 53293/ | 144.6 | A | 205.9 | P | 298.2 | P | 372 |
| | | ug = Mm.12847/len = 1805/STRA = for | | | | | | | |
| 19 | 103367_at | Cluster Incl U18975 Ganglioside expression 2/cds = (3,1604)/gb = U18975/gi = 945034/ug = Mm 1853/len = 2109/STRA = for | 28.2 | A | 28.1 | A | 94.6 | P | 125.3 |
| 19 | 93108_at | Cluster Incl J05185 Prolyl 4-hydroxylase, beta polypeptide/cds = (129,1658)/gb = J05185/gi = 200280/ug = Mm.16660/ | 293.2 | A | 180.8 | A | 442.2 | A | 458.2 |
| | | len = 2537/STRA = for | | | | | | | |
| 19 | 94902_at | Cluster Incl U38261 Mus musculus extracellular superoxide dismutase (SOD3) mRNA, complete cds/cds = (269,1024)/ | 229.6 | A | 342.9 | A | 400 | P | 462.8 |
| | | gb = U38261/gi = 1915962/ug = Mm 2407/len = 1834/STRA = for | | | | | | | |
| 19 | 97830_at | Cluster Incl L06039 Platelet/endothelial cell adhesion molecule/cds = (98,2281)/gb = L06039/gi = 453288/ug = Mm.2822/ | 140 | A | 150.9 | A | 289.8 | P | 252.5 |
| | | len = 2534/STRA = for | | | | | | | |
| 19 | 102046_at | Cluster Incl AF041847 Mus musculus cardiac ankynn repeal protein MCARP mRNA, complete cds/cds = (14,973)/ | 3.2 | A | −1.3 | A | 83.5 | P | 71.4 |
| | | gb = AF041847/gi = 2905615/ug = Mm 10279/len = 1026/STRA = for | | | | | | | |
| 19 | 103068_at | Cluster Incl U68535: Mus musculus aldo-keto reductase mRNA, complete cds/cds = (27,932)/gb = U68535/gi = 1698717/ | −39.2 | A | 55.6 | A | 81 | A | 66.1 |
| | | ug = Mm 16790/len = 1675/STRA = for | | | | | | | |
| 19 | 102195_at | Cluster Incl U88984: Mus musculus NIK mRNA, complete cds/cds = (0,3701)/gb = U88984/gi = 1872545/ug = Mm 987/ | 48 | A | 193.6 | A | 186.1 | A | 275.5 |
| | | len = 3915/STRA = rev | | | | | | | |
| 19 | 103213_at | Cluster Incl AJ245402: Mus musculus partial cdh15 gene for cadherin/cds = (0,239)/gb = AJ245402/gi = 6010257/ | 25.4 | A | 123.9 | M | 123.8 | A | 179.3 |
| | | ug = Mm 1976/len = 240/STRA = for | | | | | | | |
| 19 | 92738_at | Cluster Incl D49921: Mouse mRNA for glial cell line-derived neurotrophic factor(GDNF), complete cds/cds = (0,635)/ | 177.4 | M | 189.5 | A | 252.3 | M | 213.9 |
| | | gb = D49921/gi = 758584/ug = Mm 4679/len = 641/STRA = for | | | | | | | |
| 19 | 97166_at | Cluster Incl W49316: mc89e12 r1 Mus musculus cDNA, 5 end/clone = IMAGE-355726/clone_end = 5/gb = W49316/ | 32.7 | A | 0.8 | A | 55.4 | P | 44.8 |
| | | gi = 1337600/ug = Mm.35552/len = 475/STRA = rev | | | | | | | |
| 19 | 94643_at | Cluster Incl U35836: Tumor-associated antigen 1/cds = (10,1257)/gb = U35836/gi = 1017751/ug = Mm 8071/len = 1338/ | 51.6 | A | 58.2 | P | 91.3 | P | 63.9 |
| | | STRA = for | | | | | | | |
| 20 | 92540_f_at | Cluster Incl Z67748 M musculus spermidine synthase gene/cds = (75,983)/gb = Z67748/gi = 1061191/ug = Mm.10/len = 1261/ | 398.3 | P | 360 | P | 325.8 | P | 496 |
| | | STRA = for | | | | | | | |
| 20 | 92770_at | Cluster Incl X66449 Calcyclin/cds = (159,428)/gb = X66449/gi = 50271/ug = Mm 1221/len = 551/STRA = for | 5157.2 | P | 5280.1 | P | 2666.1 | P | 5677.6 |
| 20 | 92817_at | Cluster Incl AW120646 UI-M-BH2 3-anz-f-08-0-UI s1 Mus musculus cDNA, 3 end/clone = UI-M-BH2.3-anz-f-08-0-UI/ | 857.5 | P | 846.5 | P | 839.6 | P | 894.8 |
| | | clone_end = 3/gb = AW120646/gi = 6096019/ug = Mm 12864/len = 434/STRA = for | | | | | | | |
| 20 | 92861_i_at | Cluster Incl M13805 Mouse type I epidermal keratin mRNA, clone pkSCC-50, 3 end/cds = (0,617)/gb = M13805/gi = 198632/ | 3228.5 | P | 3531.2 | P | 2783.7 | P | 4577.5 |
| | | ug = Mm 14046/len = 801/STRA = for | | | | | | | |
| 20 | 92862_f_at | Cluster Incl M13805 Mouse type I epidermal keratin mRNA, clone pkSCC-50, 3 end/cds-(0,617)/gb = M13805/gi = 198632/ | 4125 | P | 4149.8 | P | 2478 | P | 5463.4 |
| | | ug = Mm.14046/len = 801/STRA = for | | | | | | | |
| 20 | 92877_at | Cluster Incl L19932 Transforming growth factor, beta induced, 68 kDa/cds = (45,2096)/gb = L19932/gi = 451131/ | 454.9 | P | 600.7 | P | 687.7 | P | 1004 |
| | | ug = Mm 14455/len = 2674/STRA = for | | | | | | | |
| 20 | 93018_at | Cluster Incl AI790103 ue64f04 r1 Mus musculus cDNA, 5 end/clone = IMAGE-1495903/clone_end = 5/gb = AI790103/ | 592.8 | P | 763.5 | P | 835.5 | P | 1392.4 |
| | | gi = 5337819/ug = Mm 14747/len = 346/STRA = for | | | | | | | |
| 20 | 93042_at | Cluster Incl D21207 Benzodiazepine receptor, peripheral/cds = (65,574)/gb = D21207/gi = 484053/ug = Mm 1508/len = 856/ | 1945.4 | P | 2068.4 | P | 1730.4 | P | 2448 |
| | | STRA = for | | | | | | | |
| 20 | 93078_at | Cluster Incl X04653 Mouse mRNA for Ly-6 alloantigen (Ly-6E 1)/cds = (88,492)/gb = X04653/gi = 52958/ug = Mm 15889/ | 2126.2 | P | 2282.2 | P | 1795.8 | P | 2856.8 |
| | | len = 876/STRA = for | | | | | | | |
| 20 | 93264_at | Cluster Incl AI843895 UI-M-AK1-aeu-f-09-0-UI s1 Mus musculus cDNA, 3 end/clone = UI-M-AK1-aeu-f-09-0-UI/ | 696.9 | P | 720.5 | P | 491.3 | P | 953.7 |
| | | clone_end = 3/gb = AI843895/gi = 5478108/ug = Mm 17253/len = 420/STRA = for | | | | | | | |
| 20 | 93288_at | Cluster Incl AI835883 UI-M-AI0-aat-f-08-0-UI.s1 Mus musculus cDNA, 3 end/clone = UI-M-AI0-aat-f-08-0-UI/clone_end = 3/ | 3371.5 | P | 3464.1 | P | 2842.2 | P | 3667 |
| | | gb = AI835883/gi = 5470096/ug = Mm 17912/len = 426/STRA = for | | | | | | | |
| 20 | 93362_at | Cluster Incl U27106 Adapter-related protein complex AP-2, mu1/cds = (141,1448)/gb = U27106/gi = 2065471/ug = Mm.18946/ | 1989.7 | P | 2025.3 | P | 1904.1 | P | 2279.5 |
| | | len = 2066/STRA = rev | | | | | | | |
| 20 | 93573_at | Cluster Incl V00835 Metallothionein 1/cds = (0,185)/gb = V00835/gi = 53247/ug = Mm 2041/len = 186/STRA = for | 1910.6 | P | 1639.8 | P | 2780.4 | P | 3474.5 |
| 20 | 93586_at | Cluster Incl AJ002307: Mus musculus mRNA for synaptogyrin 2/cds = (4,678)/gb = AJ002307/gi = 2959869/ug = Mm.2079/ | 245.8 | P | 514.3 | P | 85.6 | P | 447.6 |
| | | len = 1512/STRA = for | | | | | | | |
| 20 | 93605_r_at | Cluster Incl AF061260 Mus musculus immunosuperfamily protein B12 mRNA, complete cds/cds = (308,1195)/ | 170.6 | P | 205.9 | P | 191.3 | P | 225.6 |
| | | gb = AF081260/gi = 3779241/ug = Mm 20921/len = 1861/STRA = for | | | | | | | |
| 20 | 93797_g_at | Cluster Incl AW123952 UI-M-BH2 3-aqc-d-05-0-UI s1 Mus musculus cDNA, 3 end/clone = UI-M-BH2 3-aqc-d-05-0-UI/ | 272.9 | P | 313.5 | P | 312.8 | P | 476.3 |
| | | clone_end = 3/gb = AW123952/gi = 6099482/ug = Mm 2140/len = 370/STRA = for | | | | | | | |
| 20 | 94011_at | Cluster Incl AI845182 UI-M-BG0-aht-a-11-0-UI s1 Mus musculus cDNA, 3 end/clone = UI-M-BG0-aht-a-11-0-UI/ | 1172.5 | P | 1256 | P | 818.2 | P | 1742.5 |
| | | clone_end = 3/gb = AI845182/gi = 5489088/ug = Mm 21819/len = 325/STRA = for | | | | | | | |
| 20 | 94043_at | Cluster Incl AB031290 Mus musculus mRNA for C7-1 protein, complete cds/cds = (0,1391)/gb = AB031290/gi = 5771446/ | 531.4 | P | 482.3 | P | 587 | P | 587.1 |
| | | ug = Mm 21961/len = 1524/STRA = for | | | | | | | |
| 20 | 94054_at | Cluster Incl AI841808: UI-M-AN0-acl-h-05-0-UI s1 Mus musculus cDNA, 3 end/clone = UI-M-AN0-acl-h-05-0-UI/ | 775.2 | P | 638.5 | P | 716.8 | P | 948.9 |
| | | clone_end = 3/gb = AI841808/gi = 5476021/ug = Mm 22024/len = 173/STRA = for | | | | | | | |
| 20 | 94302_at | Cluster Incl AF013099 Mus musculus multiubiquitin-chain-binding protein (Mcb1) mRNA, complete cds/cds = (12,1142)/ | 868.6 | P | 955.3 | P | 818.4 | P | 1008.5 |
| | | gb = AF013099/gi = 2293572/ug = Mm 2261/len = 1252/STRA = for | | | | | | | |
| 20 | 94826_at | Cluster Incl Y11460 Mus musculus mRNA for b4 Integnn interactor, partial/cds = (0,254)/gb = Y11460/gi = 2910994/ | 881 | P | 1078.5 | P | 1045.4 | P | 1616.6 |
| | | ug = Mm 22731/len = 438/STRA = for | | | | | | | |
| 20 | 94831_at | Cluster Incl M65270 Mouse cathepsin B gene/cds = (25,1044)/gb = M65270/gi = 309151/ug = Mm.22753/len = 1848/ | 2552.8 | P | 2491 | P | 2313.1 | P | 2868.7 |
| | | STRA = for | | | | | | | |
| 20 | 94835_f_at | Cluster Incl M28739 Tubulin, beta 2/cds = (0,302)/gb = M28739/gi = 202226/ug = Mm 22774/len = 488/STRA = for | 1647.1 | P | 1533.5 | P | 1817.2 | P | 3104.8 |
| 20 | 94837_at | Cluster Incl U67328 Mus musculus NIPI-like protein (NIPIL(A3)) mRNA, complete cds/cds = (466,1437)/gb = U67328/ | 2517 | P | 3199 | P | 2881.9 | P | 3210.4 |
| | | gi = 1762985/ug = Mm 22776/len = 1720/STRA = for | | | | | | | |
| 20 | 95406_at | Cluster Incl AW125347 UI-M-BH2 1-apy-h-03-0-UI s1 Mus musculus cDNA, 3 end/clone = UI-M-BH2 1-apy-h-03-0-UI/ | 918.7 | P | 943.9 | P | 665.1 | P | 1298.4 |
| | | clone_end = 3/gb = AW125347/gi = 6100877/ug = Mm 24219/len = 331/STRA = for | | | | | | | |
| 20 | 95417_at | Cluster Incl AI117848: uc41c02.r1 Mus musculus cDNA, 5 end/clone = IMAGE-1400546/clone_end = 5/gb = AI117848/ | 289.7 | P | 273.4 | P | 300.8 | P | 388.5 |
| | | gi = 3518172/ug = Mm 24293/len = 431/STRA = for | | | | | | | |
| 20 | 95433_at | Cluster Incl AW049776 UI-M-BH1-anm-c-12-0-UI s1 Mus musculus cDNA, 3 end/clone = UI-M-BH1-anm-c-12-0-UI/ | 319.7 | P | 404.5 | P | 355.4 | P | 323.3 |
| | | clone_end = 3/gb = AW049776/gi = 5910305/ug = Mm.24495/len = 403/STRA = for | | | | | | | |
| 20 | 95511_at | Cluster Incl X69902 Integrin alpha 6/cds = (181,3402)/gb = X69902/gi = 408127/ug = Mm 25232/len = 3756/STRA = for | 134.1 | P | 210.7 | P | 259.3 | P | 448 |
| 20 | 96033_at | Cluster Incl Z22532 Syndecan 1/cds = (240,1175)/gb = Z22532/gi = 1946227/ug = Mm 2580/len = 3054/STRA = for | 349.3 | P | 339 | P | 309.3 | P | 614.6 |
| 20 | 96042_at | Cluster Incl L35528: Mus musculus manganese superoxide dismutase (MnSOD) gene/cds = (70,738)/gb = L35528/ | 197.3 | P | 258.7 | P | 266.9 | P | 497.8 |
| | | gi = 2182263/ug = Mm 2597/len = 906/STRA = for | | | | | | | |
| 20 | 96075_at | Cluster Incl AW060876 UI-M-BH1-amx-g-07-0-UI s1 Mus musculus cDNA, 3 end/clone = UI-M-BH1-amx-g-07-0-UI/ | 1130 | P | 1278.6 | P | 1262.7 | P | 1614.1 |
| | | clone_end = 3/gb = AW060876/gi = 6008627/ug = Mm 2654/len = 472/STRA = for | | | | | | | |
| 20 | 96092_at | Cluster Incl M96827 C57BL/6J ob/ob haptoglobin mRNA, complete cds/cds = (48,1091)/gb = M96827/gi = 193753/ | 945.1 | P | 1513.2 | P | 1441.4 | P | 4452.4 |
| | | ug = Mm 26730/len = 1267/STRA = for | | | | | | | |
| 20 | 96603_at | Cluster Incl AW123556 UI-M-BH2 1-aqb-g-04-0-UI s1 Mus musculus cDNA, 3 end/clone = UI-M-BH2 1-aqb-g-04-0-UI/ | 257.9 | P | 420.9 | P | 351.5 | P | 455.1 |
| | | clone_end = 3/gb = AW123556/gi = 6099086/ug = Mm 27035/len = 370/STRA = for | | | | | | | |
| 20 | 96623_at | Cluster Incl AI853172 UI-M-BH0-ajh-b-06-0-UI s1 Mus musculus cDNA, 3 end/clone = UI-M-BH0-ajh-b-06-0-UI/ | 233.4 | P | 220.1 | P | 217 | P | 471.3 |
| | | clone_end = 3/gb = AI853172/gi = 5497078/ug = Mm 27173/len = 435/STRA = for | | | | | | | |
| 20 | 96650_at | Cluster Incl AI837724: UI-M-AK0-adj-f-11-0-UI s1 Mus musculus cDNA, 3 end/clone = UI-M-AK0-adj-f-11-0-UI/clone_end = 3/ | 366.6 | P | 217.8 | P | 293.9 | P | 323.8 |
| | | gb = AI837724/gi = 5471937/ug = Mm 2733/len = 370/STRA = for | | | | | | | |
| 20 | 96657_at | Cluster Incl L10244 Spermidine/spermine N1-acetyl transferase/cds = (89,604)/gb = L10244/gi = 309503/ug = Mm 2734/ | 979.1 | P | 680.1 | P | 613.8 | P | 2078.9 |
| | | len = 961/STRA = for | | | | | | | |
| 20 | 96704_at | Cluster Incl AF058798 Mus musculus 14-3-3 protein sigma mRNA, complete cds/cds = (163,909)/gb = AF058798/ | 3751.4 | P | 3479.4 | P | 3781.1 | P | 4855.8 |
| | | gi = 3065926/ug = Mm 2758/len = 1430/STRA = for | | | | | | | |
| 20 | 96718_at | Cluster Incl AB012727 Mus musculus C184L-22 mRNA, complete cds/cds = UNKNOWN/gb = AB012727/gi = 6469043/ | 575.2 | P | 447.1 | P | 424.4 | P | 685.8 |
| | | ug = Mm 27657/len = 2532/STRA = for | | | | | | | |
| 20 | 97199_at | Cluster Incl AI854767 UI-M-BH0-aka-c-06-0-UI s1 Mus musculus cDNA, 3 end/clone = UI-M-BH0-aka-c-06-0-UI/ | 515.8 | P | 511.2 | P | 462.3 | P | 562.2 |
| | | clone_end = 3/gb = AI854767/gi = 5498673/ug = Mm 27660/len = 203/STRA = for | | | | | | | |
| 20 | 97803_at | Cluster Incl U38196 Membrane protein, palmitoylated (55 kDa)/cds = (180,1580)/gb = U38196/gi = 1616986/ug = Mm 2814/ | 694.9 | P | 644.3 | P | 641.7 | P | 812.7 |
| | | len = 1976/STRA = for | | | | | | | |
| 20 | 97875_at | Cluster Incl AW123694: UI-M-BH2.1-apl-c-08-0-UI s1 Mus musculus cDNA, 3 end/clone = UI-M-BH2 1-apl-c-08-0-UI/ | 339.6 | P | 447.1 | P | 503.1 | P | 434.2 |
| | | clone_end = 3/gb = AW123694/gi = 6099224/ug = Mm 28353/len = 479/STRA = for | | | | | | | |
| 20 | 97882_at | Cluster Incl AB032902: Mus musculus Sec61 mRNA, complete cds/cds = ( 120,1550)/gb = AB032902/gi = 6012185/ | 1158.8 | P | 1314.2 | P | 1148.8 | P | 1397.8 |
| | | ug = Mm 28375/len = 1804/STRA = rev | | | | | | | |
| 20 | 97890_at | Cluster Incl AW046181: UI-M-BH1-akw-d-06-0-UI s1 Mus musculus cDNA, 3 end/clone = UI-M-BH1-akw-d-06-0-UI/ | 705.1 | P | 473.1 | P | 1023.3 | P | 1309.3 |
| | | clone_end = 3/gb = AW046181/gi = 5906710/ug = Mm 28405/len = 376/STRA = for | | | | | | | |
| 20 | 97914_at | Cluster Incl D17666 Mouse gene for mitochondrial stress-70 protein (PBP74/CSA)/cds = (73,2112)/gb = D17666/gi = 903308/ | 1107.5 | P | 1178.5 | P | 905.6 | P | 1271.6 |
| | | ug = Mm 2849/len = 2998/STRA = for | | | | | | | |
| 20 | 98911_at | Cluster Incl AI837528: UI-M-AL0-abs-b-09-0-UI.s1 Mus musculus cDNA, 3 end/clone = UI-M-AL0-abs-b-09-0-UI/ | 93.1 | P | 146 | P | 208.7 | P | 318.5 |
| | | clone_end = 3/gb = AI837528/gi = 5471741/ug = Mm 28598/len = 340/STRA = for | | | | | | | |
| 20 | 94445_at | Cluster Incl AW125273: UI-M-BH2.1-aps-h-07-0-UI s1 Mus musculus cDNA, 3 end/clone = UI-M-BH2 1-aps-h-07-0-UI/ | 1033 | P | 1221.3 | P | 983.4 | P | 1340 |
| | | clone_end = 3/gb = AW125273/gi = 6100803/ug = Mm 28777/len = 414/STRA = for | | | | | | | |
| 20 | 94450_at | Cluster Incl AI854202 UI-M-BH0-ajz-d-08-0-UI.s1 Mus musculus cDNA, 3 end/clone = UI-M-BH0-ajz-d-08-0-UI/ | 579.1 | P | 604.3 | P | 454.6 | P | 591.5 |
| | | clone_end = 3/gb = AI854202/gi = 5498108/ug = Mm 28786/len = 543/STRA = for | | | | | | | |
| 20 | 94486_at | Cluster Incl AW125178: UI-M-BH2.1-apz-g-02-0-UI s1 Mus musculus cDNA, 3 end/clone = UI-M-BH2 1-apz-g-02-0-UI/ | 1390.6 | P | 1604.5 | P | 1504.2 | P | 1985.1 |
| | | clone_end = 3/gb = AW125178/gi = 6100708/ug = Mm 28892/len = 249/STRA = for | | | | | | | |
| 20 | 94492_at | Cluster Incl AB025406 Mus musculus mRNA for sid23p, complete cds/cds = (34,531)/gb = AB025406/gi = 5931560/ | 3930.5 | P | 3660 | P | 3284.2 | P | 4957.4 |
| | | ug = Mm 28919/len = 1639/STRA = for | | | | | | | |
| 20 | 95079_at | Cluster Incl M57683 Platelet derived growth factor receptor, alpha polypeptide/cds = (78,3347)/gb = M57683/gi = 199783/ | 255 | P | 437.9 | P | 337.2 | P | 809.4 |
| | | ug = Mm 2924/len = 3564/STRA = for | | | | | | | |
| 20 | 95102_at | Cluster Incl AW123754 UI-M-BH2.1-apm-a-08-0-UI s1 Mus musculus cDNA, 3 end/clone = UI-M-BH2.1-apm-a-08-0-UI/ | 886.9 | P | 888.4 | P | 898 | P | 1509.6 |
| | | clone_end = 3/gb = AW123754/gi = 6099284/ug = Mm 29345/len = 395/STRA = for | | | | | | | |
| 20 | 95135_at | Cluster Incl AI844396 UI-M-AL1-ahp-e-05-0-UI s1 Mus musculus cDNA, 3 end/clone = UI-M-AL1-ahp-e-05-0-UI/ | 693.3 | P | 584.6 | P | 485.7 | P | 776.9 |
| | | clone_end = 3/gb = AI844396/gi = 5488302/ug = Mm 29429/len = 432/STRA = for | | | | | | | |
| 20 | 95657_f_at | Cluster Incl AW125164 UI-M-BH2.1-apz-e-09-0-UI s1 Mus musculus cDNA, 3 end/clone = UI-M-BH2 1-apz-e-09-0-UI/ | 474.9 | P | 550.4 | P | 360.4 | P | 645.7 |
| | | clone_end = 3/gb = AW125164/gi = 6100694/ug = Mm 29541/len = 210/STRA = for | | | | | | | |
| 20 | 95688_at | Cluster Incl Y08460: Degenerative spermatocyte homolog (Drosophila)/cds = (41,1012)/gb = Y08460/gi = 2225940/ | 886.1 | P | 1263.5 | P | 1021.2 | P | 1400 |
| | | ug = Mm 29648/len = 1778/STRA = for | | | | | | | |
| 20 | 96245_at | Cluster Incl AW124530: UI-M-BH2 1-apo-g-09-0-UI s1 Mus musculus cDNA, 3 end/clone = UI-M-BH2 1-apo-g-09-0-UI/ | 261.7 | P | 228 | P | 259.8 | P | 464.4 |
| | | clone_end = 3/gb = AW124530/gi = 6100060/ug = Mm 29810/len = 487/STRA = for | | | | | | | |
| 20 | 96252_at | Cluster Incl AJ005073 Mus musculus mRNA for Alix (ALG-2-Interacting protein X), complete CDS/cds = (0,2609)/ | 753.3 | P | 636.5 | P | 798.8 | P | 849.5 |
| | | gb = AJ005073/gi = 3550455/ug = Mm 29816/len = 2610/STRA = for | | | | | | | |
| 20 | 96340_at | Cluster Incl AW124185 UI-M-BH2 1-aph-a-04-0-UI s1 Mus musculus cDNA, 3 end/clone = UI-M-BH2 1-aph-a-04-0-UI/ | 409.8 | P | 403 | P | 305.9 | P | 440.4 |
| | | clone_end = 3/gb = AW124185/gi = 6099715/ug = Mm 29987/len = 399/STRA = for | | | | | | | |
| 20 | 97451_at | Cluster Incl AI837599 UI-M-AK0-adg-a-10-0-UI s1 Mus musculus cDNA, 3 end/clone = UI-M-AK0-adg-a-10-0-UI/ | 194.6 | P | 286 | P | 370.3 | P | 759.5 |
| | | clone_end = 3/gb = AI837599/gi = 5471812/ug = Mm 30251/len = 449/STRA = for | | | | | | | |
| 20 | 97529_at | Cluster Incl AJ002390 Annexin 8/cds = (53,1036)/gb = AJ002390/gi = 2612794/ug = Mm 3267/len = 1888/STRA = for | 1379.6 | P | 1410.8 | P | 1533.7 | P | 2741.5 |
| 20 | 98114_at | Cluster Incl AF003348 Niemann Pick type C1/cds = (123,3959)/gb = AF003348/gi = 2251241/ug = Mm 3484/len = 5029/ | 371 | P | 295.5 | P | 369.4 | P | 396 |
| | | STRA = for | | | | | | | |
| 20 | 98130_at | Cluster Incl U85089 Mus musculus thioredoxin mRNA, nuclear gene encoding mitochondrial protein, complete cds/ | 917.9 | P | 968 | P | 781.6 | P | 988 9 |
| | | cds = (51,551)/gb = U85089/gi = 1814410/ug = Mm 3533/len = 1182/STRA = for | | | | | | | |
| 20 | 98153_at | Cluster Incl L20509 Chaperonin subunit 3 (gamma)/cds = (83,1717)/gb = L20509/gi = 347838/ug = Mm 3576/len = 1937/ | 963.2 | P | 865.5 | P | 734.5 | P | 1102.5 |
| | | STRA = for | | | | | | | |
| 20 | 98600_at | Cluster Incl U41341: Mus musculus endothelial monocyte-activating polypeptide I mRNA, complete cds/cds = (0,296)/ | 3104.4 | P | 3566.8 | P | 3540.9 | P | 4164.1 |
| | | gb = U41341/gi = 1150723/ug = Mm.383/len = 331/STRA = for | | | | | | | |
| 20 | 98608_at | Cluster Incl AI845886 UI-M-AO1-aeg-b-09-0-UI.s1 Mus musculus cDNA, 3 end/clone = UI-M-AO1-aeg-h-09-0-UI/ | 1160.6 | P | 1221.3 | P | 1146.1 | P | 1195.7 |
| | | clone_end = 3/gb = AI845886/gi = 5489792/ug = Mm.3845/len = 447/STRA = rev | | | | | | | |
| 20 | 98628_f_at | Cluster Incl AF003695: Hypoxia Inducible factor 1, alpha subunit/cds = (257,2767)/gb = AF003695/gi = 2197094/ | 1338.5 | P | 1330.9 | P | 1255 | P | 2037.9 |
| | | ug = Mm 3879/len = 3973/STRA = for | | | | | | | |
| 20 | 98629_f_at | Cluster Incl Y09085 Hypoxia inducible factor 1, alpha subunit/cds = (153,2585)/gb = Y09085/gi = 2113880/ug = Mm 3879/ | 1423.7 | P | 1370.4 | P | 1185.2 | P | 1984.5 |
| | | len = 3774/STRA = for | | | | | | | |
| 20 | 99087_at | Cluster Incl AW060179: UI-M-BH1-amu-d-09-0-UI s1 Mus musculus cDNA, 3 end/clone = UI-M-BH1-amu-d-09-0-UI/ | 695.4 | P | 731.3 | P | 798.2 | P | 792.5 |
| | | clone_end = 3/gb = AW060179/gi = 6007945/ug = Mm 38994/len = 244/STRA = rev | | | | | | | |
| 20 | 99101_at | Cluster Incl AB012580 Mus musculus mRNA for elF3 p66, complete cds/cds = (96,1739)/gb = AB012580/gi = 2992163/ | 902.4 | P | 1110.6 | P | 879.9 | P | 1197 |
| | | ug = Mm 3955/len = 1849/STRA = for | | | | | | | |
| 20 | 99182_at | Cluster Incl AI846712: UI-M-AN1-afi-g-12-0-UI s1 Mus musculus cDNA, 3 end/clone = UI-M-AN1-afi-g-12-0-UI/ | 564.2 | P | 300.3 | P | 366 | P | 551.9 |
| | | clone_end = 3/gb = AI846712/gi = 5490618/ug = Mm 41801/len = 381/STRA = rev | | | | | | | |
| 20 | 100051_at | Cluster Incl U17297 Erythrocyte protein band 7 2/cds = (90,944)/gb = U17297/gi = 972906/ug = Mm.4441/len = 2821/ | 942.5 | P | 806.4 | P | 1023.9 | P | 1552.3 |
| | | STRA = for | | | | | | | |
| 20 | 100123_f_at | Cluster Incl X15202: Mouse mRNA for fibronectin receptor beta-chain (VLA5-homolog)/cds = (0,2398)/gb = X15202/ | 2776.6 | P | 3202 | P | 2891.6 | P | 3665 |
| | | gi = 50986/ug = Mm 4712/len = 3491/STRA = for | | | | | | | |
| 20 | 100124_r_at | Cluster Incl X15202: Mouse mRNA for fibronectin receptor beta-chain (VLA5-homolog)/cds = (0,2398)/gb = X15202/ | 982.3 | P | 1075.5 | P | 1169.6 | P | 1563.7 |
| | | gi = 50986/ug = Mm 4712/len = 3491/STRA = for | | | | | | | |
| 20 | 100127_at | Cluster Incl M35523 Cellular retinoic acid binding protein II/cds = (115,531)/gb = M35523/gi = 192705/ug = Mm 4757/len = 868/ | 136.7 | P | 151.4 | P | 141 | P | 326.4 |
| | | STRA = for | | | | | | | |
| 20 | 100142_at | Cluster Incl X95480: DiGeorge syndrome gene c/cds = (133,1779)/gb = X95480/gi = 1550786/ug = Mm 4891/len = 2987/ | 275.1 | P | 315.6 | P | 244.5 | P | 284.7 |
| | | STRA = for | | | | | | | |
| 20 | 100154_at | Cluster Incl AIB36367: UI-M-AP0-abi-b-11-0-UI s1 Mus musculus cDNA, 3 end/clone = UI-M-AP0-abi-b-11-0-UI/ | 333.6 | P | 277.8 | P | 420.5 | P | 580.2 |
| | | clone_end = 3/gb = AI836367/gi = 5470580/ug = Mm 4975/len = 332/STRA = for | | | | | | | |
| 20 | 100535_at | Cluster Incl U76112 Mus musculus translation repressor NAT1 mRNA, complete cds/cds = (306,3026)/gb = U76112/ | 398.8 | P | 452.4 | P | 359.7 | P | 506.6 |
| | | gi = 1903415/ug = Mm 525/len = 3789/STRA = for | | | | | | | |
| 20 | 100581_at | Cluster Incl U59807 Cystatin B/cds = (0,296)/gb = U59807/gi = 1439569/ug = Mm 6095/len = 297/STRA = for | 2922.6 | P | 2960.4 | P | 2499.6 | P | 3694.1 |
| 20 | 100597_at | Cluster Incl AW049730: UI-M-BH1-ani-g-03-0-UI s1 Mus musculus cDNA, 3 end/clone = UI-M-BH1-ani-g-03-0-UI/ | 256.1 | P | 341.2 | P | 285.5 | P | 251.9 |
| | | clone_end = 3/gb = AW049730/gi = 5910259/ug = Mm 6375/len = 348/STRA = for | | | | | | | |
| 20 | 100606_at | Cluster Incl M18070 Mouse (with short incubation period) prion protein (PRNP) gene, complete cds/cds = (10,774)/ | 2753.6 | P | 2697.6 | P | 2413.1 | P | 3108.6 |
| | | gb = M18070/gi = 200528/ug = Mm 648/len = 775/STRA = for | | | | | | | |
| 20 | 101025_f_at | Cluster Incl AJ005559 Small proline-rich protein 2A/cds = (0,251)/gb = AJ005559/gi = 3093357/ug = Mm 6853/len = 252/ | 1589 | P | 1230.6 | P | 1444.9 | P | 2449.4 |
| | | STRA = for | | | | | | | |
| 20 | 101482_at | Cluster Incl D85137 Mouse mRNA for PP1gamma, complete cds/cds = (107,1078)/gb = D85137/gi = 1332408/ug = Mm 7793/ | 958.6 | P | 1044.3 | P | 1192.1 | P | 1298.9 |
| | | len = 2200/STRA = for | | | | | | | |
| 20 | 101487_f_at | Cluster Incl U47737 Mus musculus thymic shared antigen-1 (TSA-1) gene, complete cds/cds = (106,498)/gb = U47737/ | 2421.5 | P | 1873.2 | P | 2253.4 | P | 2869.9 |
| | | gi = 1199649/ug = Mm.788/len = 1117/STRA = for | | | | | | | |
| 20 | 101963_at | Cluster Incl X06086 Cathepsin L/cds = (68,1072)/gb = X06086/gi = 53046/ug = Mm 930/len = 1374/STRA = for | 1131.4 | P | 1331.9 | P | 1016.7 | P | 2070.6 |
| 20 | 101993_at | Cluster Incl X56304 Tenascin C/cds = (126,6185)/gb = X56304/gi = 54768/ug = Mm 980/len = 6831/STRA = for | 509.1 | P | 813.2 | P | 506.6 | P | 2177.7 |
| 20 | 102052_at | Cluster Incl AA871791 vq41a10 r1 Mus musculus cDNA, 5 end/clone = IMAGE-1096794/clone_end = 5/gb = AA871791/ | 366.3 | P | 408.6 | P | 469.2 | P | 465.2 |
| | | gi = 2967236/ug = Mm.10297/len = 292/STRA = for | | | | | | | |
| 20 | 102315_at | Cluster Incl AW124570 UI-M-BH2 1-apt-d-08-0-UI s1 Mus musculus cDNA, 3 end/clone = UI-M-BH2 1-apt-d-08-0-UI/ | 254.1 | P | 271.1 | P | 237.3 | P | 306.8 |
| | | clone_end = 3/gb = AW124570/gi = 6100100/ug = Mm 10665/len = 450/STRA = for | | | | | | | |
| 20 | 102348_at | Cluster Incl AI551087 vx36f11.x1 Mus musculus cDNA, 3 end/clone = IMAGE-1277325/clone_end = 3/gb = AI551087/ | 767 | P | 666.6 | P | 997.7 | P | 1826.7 |
| | | gi = 4483450/ug = Mm.11223/len = 446/STRA = rev | | | | | | | |
| 20 | 103035_at | Cluster Incl U60020: Transporter 1, ABC (ATP binding cassette)/cds = (0,2174)/gb = U60020/gi = 1399904/ug = Mm 16122/ | 246 | P | 327.2 | P | 266.9 | P | 432.8 |
| | | len = 2175/STRA = for | | | | | | | |
| 20 | 103059_at | Cluster Incl X93038 M musculus mRNA for MATB protein/cds = (87,353)/gb = X93038/gi = 1085067/ug = Mm 1662/len = 522/ | 2459.3 | P | 2315.7 | P | 2470 | P | 2862 |
| | | STRA = rev | | | | | | | |
| 20 | 103416_at | Cluster Incl AI844810 UI-M-AH1-agx-b-06-0-UI s1 Mus musculus cDNA, 3 end/clone = UI-M-AH1-agx-b-06-0-UI/ | 1086.2 | P | 1223.7 | P | 1232.4 | P | 1674.5 |
| | | clone_end = 3/gb = AI844810/gi = 5488716/ug = Mm 18856/len = 520/STRA = for | | | | | | | |
| 20 | 103589_at | Cluster Incl AF053235 Mus musculus keratin 16 mRNA, complete cds/cds = (12,1421)/gb = AF053235/gi = 3925354/ | 523.4 | P | 798.7 | P | 1224 | P | 4216.4 |
| | | ug = Mm.19889/len = 1580/STRA = for | | | | | | | |
| 20 | 103877_at | Cluster Incl AW060485 UI-M-BH1-ann-a-09-0-UI s1 Mus musculus cDNA, 3 end/clone = UI-M-BH1-ann-a-09-0-UI/ | 218.6 | P | 286.4 | P | 219 | P | 275.3 |
| | | clone_end = 3/gb = AW060485/gi = 6008236/ug = Mm 21184/len = 263/STRA = for | | | | | | | |
| 20 | 104074_at | Cluster Incl AW212109 uo82e01.x1 Mus musculus cDNA, 3 end/clone = IMAGE-2649048/clone_end = 3/gb = AW212109/ | 284.2 | P | 242.3 | P | 238.1 | P | 307.7 |
| | | gi = 6518110/ug = Mm 21687/len = 298/STRA = for | | | | | | | |
| 20 | 104105_at | Cluster Incl AI854665 UI-U-BH0-akc-a-09-0-UI s1 Mus musculus cDNA, 3 end/clone = UI-M-BH0-akc-a-09-0-UI/ | 436 | P | 383.4 | P | 344.2 | P | 420 |
| | | clone_end = 3/gb = AI854665/gi = 5498571/ug = Mm 21892/len = 486/STRA = for | | | | | | | |
| 20 | 104283_at | Cluster Incl AI037493 ub62a05 r1 Mus musculus cDNA, 5 end/clone = IMAGE-1382288/clone_end = 5/gb = AI037493 | 100.4 | P | 103.3 | P | 100.5 | P | 184.6 |
| | | gi = 3261184/ug = Mm 22252/len = 416/STRA = for | | | | | | | |
| 20 | 104300_at | Cluster Incl AI117936 uc41g02 r1 Mus musculus cDNA, 5 end/clone = IMAGE-1400594/clone_end = 5/gb = AI117936/ | 866.4 | P | 1074.9 | P | 694.5 | P | 1242.8 |
| | | gi = 3518260/ug = Mm 22343/len = 182/STRA = for | | | | | | | |
| 20 | 104369_at | Cluster Incl AB012033 Mus musculus gene for keratin 6 alpha/cds = (0,1661)/gb = AB012033/gi = 3882140/ug = Mm 22629/ | 2232.2 | P | 2070.9 | P | 754.2 | P | 2471.1 |
| | | len = 2259/STRA = for | | | | | | | |
| 20 | 104370_s_at | Cluster Incl K02108 Keratin complex 2, gene 6A/cds = (49,1710)/gb = K02108/gi = 198634/ug = Mm 22629/len = 2005/ | 1043.4 | P | 881.5 | P | 453 | P | 3240.8 |
| | | STRA = for | | | | | | | |
| 20 | 104386_f_at | Clusler Incl AI843901: UI-M-AK1-aeu-g-04-0-UI s1 Mus musculus cDNA, 3 end/clone = UI-M-AK1-aeu-g-04-0-UI/ | 971 | P | 1090.2 | P | 832.6 | P | 1290.4 |
| | | clone_end = 3/gb = AI843901/gi = 5478114/ug = Mm 227/len = 300/STRA = for | | | | | | | |
| 20 | 104579_r_at | Cluster Incl AI195392: ue72102.r1 Mus musculus cDNA, 5 end/clone = IMAGE-149666/clone_end = 5/gb = AI195392/ | 495.6 | P | 410 | P | 443.3 | P | 700 |
| | | gi = 3747998/ug = Mm.23961/len = 262/STRA = for | | | | | | | |
| 20 | 104605_at | Cluster Incl AW047554 UI-M-BH1-ama-g-03-0-UI s1 Mus musculus cDNA, 3 end/clone = UI-M-BH1-ama-g-03-0-UI/ | 858 | P | 1014.7 | P | 857.8 | P | 1032.5 |
| | | clone_end = 3/gb = AW047554/gi = 5908083/ug = Mm 24126/len = 312/STRA = for | | | | | | | |
| 20 | 104628_at | Cluster Incl X61172.Mouse mRNA for alpha-mannosidase II/cds = (87,3539)/gb = X61172/gi = 49943/ug = Mm 2433/ | 536.2 | P | 465.7 | P | 504.9 | P | 582.4 |
| | | len = 6080/STRA = for | | | | | | | |
| 20 | 104716_at | Cluster Incl X60367: Retinol binding protein 1, cellular/cds = UNKNOWN/gb = X60367/gi = 50547/ug = Mm 2450/len = 2633/ | 233 | P | 302.5 | P | 249.7 | P | 541.6 |
| | | STRA = for | | | | | | | |
| 20 | 94367_at | Cluster Incl AI850362 UI-M-BG1-aig-b-06-0-UI s1 Mus musculus cDNA, 3 end/clone = UI-M-BG1-aig-b-06-0-UI/ | 642 | P | 510.4 | P | 493.7 | P | 710.4 |
| | | clone_end = 3/gb = AI850362/gi = 5494268/ug = Mm 25309/len = 494/STRA = for | | | | | | | |
| 20 | 94391_at | Cluster Incl AW123650 UI-M-BH2 1-apl-g-08-0-U1.s1 Mus musculus cDNA, 3 end/clone = UI-M-BH2 1-apl-g-08-0-UI/ | 87.9 | P | 79.7 | P | 181 | P | 393.9 |
| | | clone_end = 3/gb = AW123650/gi = 6099180/ug = Mm 25652/len = 417/STRA = for | | | | | | | |
| 20 | 94433_at | Cluster Incl AW060684 UI-M-BH1-anl-e-02-0-UI s1 Mus musculus cDNA, 3 end/clone = UI-M-BH1-anl-e-02-0-UI/ | 1395.4 | P | 1312.5 | P | 1009.2 | P | 1778.7 |
| | | clone_end = 3/gb = AW060684/gi = 6008435/ug = Mm.26492/len = 383/STRA = for | | | | | | | |
| 20 | 94928_at | Cluster Incl X87128 M musculus p75 TNF receptor DNA/cds = (67,133)/gb = X87128/gi = 809043/ug = Mm 2666/len = 134/ | 186.1 | P | 123.4 | P | 120.9 | P | 184 |
| | | STRA = for | | | | | | | |
| 20 | 94991_at | Cluster Incl AW046661.UI-M-BH1-ald-g-02-0-UI s1 Mus musculus cDNA, 3 end/clone = U-M-BH1-ald-g-02-0-UI/ | 153.8 | P | 160.4 | P | 174.6 | P | 196.3 |
| | | clone_end = 3/gb = AW046661/gi = 5907190/ug = Mm 27313/len = 430/STRA = for | | | | | | | |
| 20 | 95542_at | Cluster Incl AI835858.UI-M-AI0-aat-c-09-0-UI s1 Mus musculus cDNA, 3 end/clone = UI-M-AI0-aal-c-09-0-UI/clone_end = 3/ | 2058.8 | P | 1874.7 | P | 1956.4 | P | 2504.8 |
| | | gb = AI835858/gi = 5470071/ug = Mm 27685/len = 469/STRA = rev | | | | | | | |
| 20 | 96187_at | Cluster Incl AI837021 UI-M-AP0-abj-a-11-0-UI s2 Mus musculus cDNA, 3 end/clone = UI-M-AP0-abj-a-11-0-UI/ | 479.7 | P | 471.6 | P | 491.8 | P | 786.6 |
| | | clone_end = 3/gb = AI837021/gi = 5471234/ug = Mm 28488/len = 182/STRA = for | | | | | | | |
| 20 | 97333_at | Cluster Incl AW121185 UI-M-BH2 3-aok-b-01-0-UI s1 Mus musculus cDNA, 3 end/clone = UI-M-BH2 3-aok-b-01-0-UI/ | 965.1 | P | 1173.9 | P | 941.9 | P | 1105 |
| | | clone_end = 3/gb = AW121185/gi = 6096518/ug = Mm 29547/len = 415/STRA = for | | | | | | | |
| 20 | 97383_at | Cluster Incl AI848623 UI-M-AH1-agv-d-05-0-UI s1 Mus musculus cDNA, 3 end/clone = UI-M-AH1-agv-d-05-0-UI/ | 737.9 | P | 630.1 | P | 655.7 | P | 1192.3 |
| | | clone_end = 3/gb = AI848623/gi = 5492569/ug = Mm 29762/len = 466/STRA = rev | | | | | | | |
| 20 | 97419_at | Cluster Incl AW060956 UI-M-BH1-anv-g-02-0-UI s1 Mus musculus cDNA, 3 end/clone = UI-M-BH1-anv-g-02-0-UI/ | 198.5 | P | 242.2 | P | 196.3 | P | 278.2 |
| | | clone_end = 3/gb = AW060956/gi = 6008707/ug = Mm 29985/len = 493/STRA = for | | | | | | | |
| 20 | 97930= f = at | Cluster Incl AF033620 CD151 antigen/cds = (82,843)/gb AF033620/gi = 3298592/ug = Mm 30246/len = 1618/STRA = for | 1224.2 | P | 1111.4 | P | 824.5 | P | 1229.3 |
| 20 | 97932_f_at | Cluster Incl D89290 CD151 antigen/cds = (327,1088)/gb = D89290/gi = 2447006/ug = Mm 30246/len = 1819/STRA = for | 1516.3 | P | 1458.3 | P | 1335.7 | P | 1605.9 |
| 20 | 97950_at | Cluster Incl X75129 Xanthine dehydrogenase/cds = (25,4032)/gb = X75129/gi = 473040/ug = Mm 3043/len = 4554/STRA = for | 144.4 | P | 191.5 | P | 251.5 | P | 495.3 |
| 20 | 98001_at | Cluster Incl U58203 Mus musculus Lsc (lsc) oncogene mRNA, complete cds/cds = (91,2850)/gb = U58203/gi = 1389755/ | 127.6 | P | 163 | P | 149.1 | P | 195 |
| | | ug = Mm 3181/len = 3200/STRA = for | | | | | | | |
| 20 | 98402_at | Cluster Incl AI843799 UI-M-AN1-afc-e-09-0-UI s1 Mus musculus cDNA, 3 end/clone = UI-M-AN1-afc-e-09-0-UI/ | 826.8 | P | 689.1 | P | 719.1 | P | 1049.7 |
| | | clone_end = 3/gb = AI843799/gi = 5478012/ug = Mm 3350/len = 402/STRA = for | | | | | | | |
| 20 | 98418_at | Cluster Incl U10115-Dishevelled, dsh homolog (Drosophila)/cds = (262,2349)/gb = U10115/gi = 497689/ug = Mm 3400/ | 449.9 | P | 413.1 | P | 406.4 | P | 510.3 |
| | | len = 3286/STRA = for | | | | | | | |
| 20 | 98423_at | Cluster Incl M81445 Gap junction membrane channel protein beta 2/cds = (0,680)/gb = M81445/gi = 192844/ug = Mm 34118/ | 3371.6 | P | 2800.2 | P | 2674.4 | P | 3669.7 |
| | | len = 681/STRA = for | | | | | | | |
| 20 | 99057_at | Cluster Incl M12379 Mouse Thy-1 2 glycoprotein gene, complete cds/cds = (0,488)/gb = M12379/gi = 202040/ug = Mm 3951/ | 750.3 | P | 1399.8 | P | 1029.6 | P | 1441 |
| | | len = 489/STRA = for | | | | | | | |
| 20 | 100428_at | Cluster Incl U43327: Laminin, gamma 2/cds = (39,3617)/gb = U43327/gi = 1151216/ug = Mm 4717/len = 5159/STRA = for | 785.1 | P | 1019.4 | P | 1089.1 | P | 2409.3 |
| 20 | 100445_f_at | Cluster Incl X91825 M musculus mRNA for SPRR1b protein/cds = (45,506)/gb = X91825/gi = 1213026/ug = Mm 4826/ | 404 | P | 692.3 | P | 491.8 | P | 5719 |
| | | len = 688/STRA = for | | | | | | | |
| 20 | 100472_at | Cluster Incl D10727: Mus musculus mRNA for NDPP-1 protein, complete cds/cds = (857,2026)/gb = D10727/gi = 220499/ | 320.6 | P | 346.8 | P | 428 | P | 475.4 |
| | | ug = Mm 4941/len = 4308/STRA = rev | | | | | | | |
| 20 | 100499_at | Cluster Incl D29797 Mouse mRNA for syntaxin 3A, complete cds/cds = (103,972)/gb = D29797/gi = 924267/ug = Mm 5122/ | 248.9 | P | 210.7 | P | 232.9 | P | 319.9 |
| | | len = 1073/STRA = for | | | | | | | |
| 20 | 100946_at | Cluster Incl AF109906 Mus musculus MHC class III region RD gene, partial cds, Bf, C2, G9A, NG22, G9, HSP70, HSP70, | 1765.2 | P | 1519.5 | P | 1265 | P | 1452.1 |
| | | HSC701, and smRNP genes, complete cds, G7A gene, partial cds, and unknown genes/cds = (0,1928)/gb = AF109906/ | | | | | | | |
| | | gi = 3986763/ug = Mm 6388/len = 1929/STRA = for | | | | | | | |
| 20 | 102203_at | Cluster Incl M95545 Placental protein 11 related/cds = (9,1373)/gb = M95545/gi = 201907/ug = Mm.1001/len = 2407/ | 383.6 | P | 497.2 | P | 592.3 | P | 1175.1 |
| | | STRA = rev | | | | | | | |
| 20 | 102248_f_at | Cluster Incl Y17138.Calcium/calmodulin-dependent serine protein kinase/cds = (492,3266)/gb = Y17138/gi = 3087817/ | 381.5 | P | 290.7 | P | 310.7 | P | 427.8 |
| | | ug = Mm.10735/len = 4393/STRA = for | | | | | | | |
| 20 | 102255_at | Cluster Incl AB015978 Oncostatin receptor/cds = (779,3691)/gb = AB015978/gi = 3721859/ug = Mm 10760/len = 4026/ | 98.6 | P | 94.7 | P | 191.8 | P | 450.2 |
| | | STRA = for | | | | | | | |
| 20 | 103217_at | Cluster Incl Y14041: Mus musculus mRNA for CASH alpha protein/cds = (74,1528)/gb = Y14041/gi = 2653419/ug = Mm.1993/ | 400.5 | P | 396.3 | P | 362.4 | P | 459.6 |
| | | len = 2770/STRA = for | | | | | | | |
| 20 | 103305_at | Cluster Incl L04678 Integrin beta 4/cds = UNKNOWN/gb = L04678/gi = 192151/ug = Mm.21117/len = 5907/STRA = for | 153.6 | P | 251.3 | P | 351.5 | P | 766.5 |
| 20 | 103306_at | Cluster Incl AW259940: um80f09 y1 Mus musculus cDNA, 5 end/clone = IMAGE-2317673/clone_end = 5/gb = AW259940/ | 60.4 | P | −24.7 | P | 84.3 | P | 114.9 |
| | | gi = 6632921/ug = Mm 21134/len = 429/STRA = for | | | | | | | |
| 20 | 103448_at | Cluster Incl M83218 Mus musculus intracellular calcium-binding protein (MRP8) mRNA, complete cds/cds = (17,286)/ | 301.7 | P | 502.2 | P | 2938.9 | P | 4994.7 |
| | | gb = M83218/gi = 199809/ug = Mm 21567/len = 343/STRA = for | | | | | | | |
| 20 | 103723_at | Cluster Incl AA608387 vo42b10 r1 Mus musculus cDNA, 5 end/clone = IMAGE-1052539/clone_end = 5/gb = AA608387/ | 106.5 | P | 113.7 | P | 124.2 | P | 325.9 |
| | | gi = 2455274/ug = Mm 24208/len = 390/STRA = for | | | | | | | |
| 20 | 103930_at | Cluster lncl AI413179 mc22e12.x1 Mus musculus cDNA, 3 end/clone = IMAGE-349294/clone_end = 3/gb = AI413179/ | 253.9 | P | 167.1 | P | 416 | P | 392.4 |
| | | gi = 4256683/ug = Mm 25117/len = 446/STRA = for | | | | | | | |
| 20 | 104260_at | Cluster Incl AW123061: UI-M-BH2.1-apc-a-11-0-UI.s1 Mus musculus cDNA, 3 end/clone = UI-M-BH2.1-apc-a-11-0-UI/ | 212.9 | P | 191.9 | P | 202.6 | P | 244.3 |
| | | clone_end = 3/gb = AW123061/gi = 6098591/ug = Mm.28411/len = 288/STRA = for | | | | | | | |
| 20 | 104406_at | Cluster Incl AI060798 ub44a06 r1 Mus musculus cDNA, 5 end/clone = IMAGE-1380582/clone_end = 5/gb = AI060798/ | 256.5 | P | 243.8 | P | 378.9 | P | 533.8 |
| | | gi = 3336221/ug = Mm 28768/len = 445/STRA = for | | | | | | | |
| 20 | 104469_at | Cluster Incl M73748 Mouse OTS-8 mRNA, complete cds/cds = (166,780)/gb = M73748/gi = 200164/ug = Mm 2976/len = 1661/ | 180.9 | P | 207.4 | P | 324.2 | P | 801.3 |
| | | STRA = for | | | | | | | |
| 20 | 92978_s_at | Cluster Incl X16490 Plasminogen activator inhibitor, type II/cds = (0, 1247)/gb = X16490/gi = 53589/ug = Mm.5019/len = 1905/ | 304.8 | P | 344.5 | P | 516.5 | P | 1128.2 |
| | | STRA = for | | | | | | | |
| 20 | 93371_at | Cluster Incl U88064: Mus musculus basonuclin mRNA, complete cds/cds = (43,2928)/gb = U88064/gi = 2408218/ | 317.8 | P | 266 | P | 210.5 | P | 417.7 |
| | | ug = Mm.6110/len = 4441/STRA = for | | | | | | | |
| 20 | 93424_at | Cluster Incl AI836915 UI-M-AJ0-aav-c-11-0-UI.s1 Mus musculus cDNA, 3 end/clone = UI-M-AJ0-aav-c-11-0-UI/ | 392.6 | P | 492.3 | P | 464 | P | 617.3 |
| | | clone_end = 3/gb = AI836915/gi = 5471128/ug = Mm 6478/len = 422/STRA = rev | | | | | | | |
| 20 | 94121_at | Cluster Incl AJ005566 Small proline-rich protein 2H/cds(31,357)/gb = AJ005566/gi = 3093370/ug = Mm.10693/len = 728/ | 416.5 | P | 623.2 | P | 591 | P | 2853.6 |
| | | STRA = rev | | | | | | | |
| 20 | 94796_at | Cluster Incl AA710643 vt51d05 r1 Mus musculus cDNA, 5 end/clone = IMAGE-1166601/clone_end = 5/gb = AA710643/ | 1356.9 | P | 1587.3 | P | 1350.4 | P | 1584.9 |
| | | gi = 2720561/ug = Mm.1800/len = 254/STRA = for | | | | | | | |
| 20 | 100772_g_at | Cluster Incl AF068182 Mus musculus B cell linker protein BLNK mRNA, complete cds/cds = (132,1505)/gb = AF068182/ | 186.2 | P | 181.7 | P | 158.4 | P | 225.5 |
| | | gi = 3406752/ug = Mm.9749/len = 1719/STRA = for | | | | | | | |
| 20 | 101717_at | Cluster Incl U26176 Somatostatin receptor 4/cds = (0,1154)/gb = U26176/gi = 833840/ug = Mm 57026/len = 1155/STRA = for | 466.7 | P | 369 | P | 131.7 | P | 519.7 |
| 20 | 99701_f_at | Cluster Incl AJ005560 Small proline-rich protein 2B/cds = (0, 296)/gb = AJ005560/gi = 3093359/ug = Mm 23796/len = 297/ | 1143.2 | P | 856.1 | P | 1086 | P | 2347.3 |
| | | STRA = for | | | | | | | |
| 20 | 101621_at | Cluster Incl AF008595.Mus musculus lacrimal gland protein mRNA, complete cds/cds = (17,298)/gb = AF008595/ | 327.8 | P | 437.8 | P | 315.4 | P | 528.5 |
| | | gi = 2338287/ug = Mm 6205/len = 420/STRA = for | | | | | | | |
| 20 | 102108_f_at | Cluster Incl AI505453 vr15c02.x1 Mus musculus cDNA, 3 end/clone = IMAGE-1120706/clone_end = 3/gb = AI505453/ | 610.5 | P | 761.2 | P | 655.8 | P | 889.8 |
| | | gi = 4403304/ug = Mm 42551/len = 260/STRA = rev | | | | | | | |
| 20 | 92618_at | Cluster Incl AW125253 UI-M-BH2.1-aps-f-11-0-UI s1 Mus musculus cDNA, 3 end/clone = UI-M-BH2 1-aps-f-11-0-UI/ | 232.6 | P | 435.7 | A | 320.4 | P | 514.6 |
| | | clone_end = 3/gb = AW125253/gi = 6100783/ug = Mm.1106/len = 245/STRA = for | | | | | | | |
| 20 | 93798_at | Cluster Incl AI839988 UI-M-AH0-acs-e-11-0-UI s1 Mus musculus cDNA, 3 end/clone = UI-M-AH0-acs-e-11-0-UI/ | 555.1 | P | 865.1 | P | 712.5 | P | 920.1 |
| | | clone_end = 3/gb = AI839988/gi = 5474201/ug = Mm.2140/len = 352/STRA = for | | | | | | | |
| 20 | 96957_at | Cluster Incl AB006463 Mus musculus mRNA for KIP, complete cds/cds = (26,601)/gb = AB006463/gi = 4185596/ | 292.3 | P | 340.4 | P | 149.6 | P | 383.7 |
| | | ug = Mm.30217/len = 687/STRA = for | | | | | | | |
| 20 | 99081_at | Cluster Incl AF010254.Complement component 1 inhibitor/cds = (66, 1580)/gb = AF010254/gi = 3236325/ug = Mm 38888/ | 417.4 | P | 454.6 | P | 358.7 | P | 604.5 |
| | | len = 1775/STRA = for | | | | | | | |
| 20 | 99159_at | Cluster Incl AI842675 UI-M-AO1-aeo-b-01-0-UI s1 Mus musculus cDNA, 3 end/clone = UI-M-AO1-aeo-b-01-0-UI/ | 240.4 | P | 125.5 | P | 215 | P | 372.1 |
| | | clone_end = 3/gb = AI842675/gi = 5476888/ug = Mm.41656/len = 280/STRA = for | | | | | | | |
| 20 | 100610_at | Cluster Incl AF058298 Mus musculus calpain small subunit mRNA, complete cds/cds = (34,843)/gb = AF058298/ | 784.7 | P | 1043.1 | P | 1047 | P | 1347.2 |
| | | gi = 3511115/ug = Mm 6534/len = 1401/STRA = for | | | | | | | |
| 20 | 101024_i_at | Cluster Incl AJ005559 Small proline-rich protein 2A/cds = (0,251)/gb = AJ005559/gi = 3093357/ug = Mm.6853/len = 252/ | 1337.1 | P | 734.8 | P | 1295.3 | P | 2672 |
| | | STRA = for | | | | | | | |
| 20 | 95035_at | Cluster Incl AI467390. vd15c12 x1 Mus musculus cDNA, 3 end/clone = IMAGE-792598/clone_end = 3/gb = AI467390/ | 326.3 | P | 261.9 | P | 362.2 | P | 419.7 |
| | | gi = 4320727/ug = Mm.27546/len = 326/STRA = for | | | | | | | |
| 20 | 96207_at | Cluster Incl AW046449 UI-M-BH1-akp-e-11-0-UI s1 Mus musculus cDNA, 3 end/clone = UI-M-BH1-akp-e-11-0-UI/ | 435.6 | P | 336.6 | P | 334.2 | A | 703.2 |
| | | clone_end = 3/gb = AW046449/gi = 5906978/ug = Mm 28645/len = 373/STRA = for | | | | | | | |
| 20 | 96212_at | Cluster Incl AI853918 UI-M-BH0-aju-f-03-0-UI.s1 Mus musculus cDNA, 3 end/clone = UI-M-BH0-aju-f-03-0-UI/ | 273 | P | 261.4 | P | 246.3 | P | 329.2 |
| | | clone_end = 3/gb = AI853918/gi = 5497824/ug = Mm.28662/len = 352/STRA = for | | | | | | | |
| 20 | 96738_at | Cluster Incl U41765: Mus musculus metalloprotease/disintegrin/cysteine rich protein precursor (MDC9) cDNA, complete cds/ | 194.8 | P | 196.7 | P | 206.1 | P | 288.8 |
| | | cds = (13,2550)/gb = U41765/gi = 1235675/ug = Mm.28908/len = 3850/STRA = for | | | | | | | |
| 20 | 98018_at | Cluster Incl L39017 Protein C receptor, endothelial/cds = (55, 783)/gb = L39017/gi = 728480/ug = Mm 3243/len = 1358/ | 177.9 | P | 57.9 | M | 94.3 | P | 365.2 |
| | | STRA = for | | | | | | | |
| 20 | 98427_s_at | Cluster Incl M57999.Nuclear factor of kappa light chain gene enhancer in B-cells 1, p105/cds = (291,3206)/gb = M57999/ | 204.7 | P | 150.6 | P | 299.6 | P | 330.7 |
| | | gi = 201931/ug = Mm.3420/len = 3892/STRA = for | | | | | | | |
| 20 | 101902_at | Cluster Incl X17459 Recombining binding protein suppressor of hairless (Drosophila)/cds = (0,1580)/gb = X17459/gi = 52756/ | 162.7 | P | 184.9 | P | 86.6 | P | 178.8 |
| | | ug = Mm 89726/len = 3986/STRA = for | | | | | | | |
| 20 | 103286_at | Cluster Incl AB012611 Mus musculus Hbp mRNA for Hrs binding Protein, complete cds/cds = (64,1635)/gb = AB012611/ | 214.7 | P | 112.5 | P | 140.4 | P | 166.9 |
| | | gi = 3721584/ug = Mm 20891/len = 1780/STRA = for | | | | | | | |
| 20 | 103497_at | Cluster Incl AA592351 Vo27b01 r1 Mus musculus cDNA, 5 end/ctone-IMAGE-1051081/clone_end = 5/gb = AA592351/ | 71.9 | P | 79.3 | P | 34.3 | A | 98.3 |
| | | gi = 2406014/ug = Mm.22318/len = 131/STRA = for | | | | | | | |
| 20 | 103991_at | Cluster Incl M61704: Mouse embryonic alkaline phosphatase gene, complete cds/cds = (0, 1589)/gb = M61704/gi = 192976/ | 262.4 | P | 141.6 | P | 121.4 | P | 367.5 |
| | | ug = Mm.26096/len = 1590/STRA = for | | | | | | | |
| 20 | 103995_at | Cluster Incl AF065441: Mus musculus FGF binding protein 1 mRNA complete cds/cds = (0,755)/gb = AF065441/ | 319.1 | P | 285.8 | P | 188.4 | P | 375.1 |
| | | gi = 3153884/ug = Mm.2643/len = 756/STRA = for | | | | | | | |
| 20 | 104184_at | Cluster Incl D16497: Natnuretic peptide precursor type B/cds = (78, 443)/gb = D16497/gi = 493659/ug = Mm 2740/len = 668/ | 49.3 | P | 61.5 | P | 58.3 | P | 173.6 |
| | | STRA = for | | | | | | | |
| 20 | 92369_at | Cluster Incl M92420 Transforming growth factor alpha/cds = (5, 484)/gb = M92420/gi = 202155/ug = Mm 3992/len = 497/ | 121.8 | P | 110.5 | P | 67.5 | M | 163.9 |
| | | STRA = for | | | | | | | |
| 20 | 92474_at | Cluster Incl AF083497.untilled/cds = (31, 3255)/gb = AF083497/gi = 3986489/ug = Mm.42128/len = 3924/STRA = for | 33.1 | A | 87.7 | P | 71.5 | P | 153.8 |
| 20 | 95984_at | Cluster Incl AI586160 vu95c12.x1 Mus musculus cDNA, 3 end/clone = IMAGE-1209910/clone_end = 3/gb = AI536160/ | 102.1 | P | 114.8 | A | 59.2 | P | 159.1 |
| | | gi = Mm.25035/len = 483/STRA = rev | | | | | | | |
| 20 | 96997_at | Custer Incl Y19035-Mus musculus mRNA for amilonde-sensitive Na + channel (BLINaC gene)/cds = (28, 1515)/gb = / | 110.4 | P | 139 | P | 87.8 | A | 110.3 |
| | | gi = 5824009/ug = Mm 88839/len = 1656/STRA = for | | | | | | | |
| 20 | 100723_f_at | Cluster Incl AJ005563: Small proline-rich protein 2E/cds = (0,230)/gb = AJ005563/gi = 3093364/ug = Mm.19358/len = 231/ | 1088.3 | P | 997 | P | 1063 | P | 1768.3 |
| | | STRA = for | | | | | | | |
| 20 | 93272_at | Cluster Incl AF062076 Mus musculus palmitylated serine/threonine kinase (Psk) mRNA, complete cds/cds = (0,917)/ | 244.4 | P | 157.3 | P | 134.8 | P | 326.4 |
| | | gb = AF062076/gi = 4106341/ug = Mm.17461/len = 918/STRA = for | | | | | | | |
| 20 | 94224_s_at | Cluster Incl M74123 Mus musculus (strain C57BI/6) mRNA sequence/cds = UNKNOWN/gb = M74123/gi = 198867/ | 168.4 | P | 20.7 | P | 41.1 | P | 565.8 |
| | | ug = Mm 22262/len = 1623/STRA = for | | | | | | | |
| 20 | 94264_at | Cluster Incl AW122170 UI-M-BH2 2-aor-g-12-0-UI s1 Mus musculus cDNA, 3 end/clone = UI-M-BH2 2-aor-g-12-0-UI/ | 631.3 | P | 667.5 | A | 561.8 | P | 652 |
| | | clone_end = 3/gb = AW122170/gi = 6097613/ug = Mm 22467/len = 188/STRA = for | | | | | | | |
| 20 | 94918_at | Cluster Incl AI839392 UI-M-AN0-aco-b-02-0-UI.s1 Mus musculus cDNA, 3 end/clone = UI-M-AN0-aco-b-02-0-UI/ | 354.7 | P | 422.6 | P | 262.8 | P | 415.8 |
| | | clone_end = 3/gb = AI839392/gi = 5473605/ug = Mm 24174/len = 181/STRA = for | | | | | | | |
| 20 | 96925_at | Cluster Incl AW122429 UI-M-BH2.2-aow-g-07-0-UI.s1 Mus musculus cDNA, 3 end/clone = UI-M-BH2 2-aow-g-07-0-UI/ | 39.1 | A | −16.1 | P | 99.3 | P | 131.8 |
| | | clone_end = 3/gb = AW122429/gi = 6097940/ug = Mm.30161/len = 477/STRA = for | | | | | | | |
| 20 | 101551_s_at | Cluster Incl X78989 M. musculus mRNA for testin/cds = (120,1226)/gb = X78989/gi = 475207/ug = Mm.88645/len = 1377/ | 172 | P | 129.5 | P | 135 | P | 232.7 |
| | | STRA = for | | | | | | | |
| 20 | 94380_at | Cluster Incl AI852581 UI-M-BH0-aiu-a-11-0-UI s1 Mus musculus cDNA, 3 end/clone = UI-M-BH0-aiu-a-11-0-UI/ | 357.3 | P | 493 | P | 214.6 | A | 699.2 |
| | | clone_end = 3/gb = AI852581/gi = 5496487/ug = Mm 25576/len = 399/STRA = for | | | | | | | |
| 20 | 95591_at | Cluster Incl AI843335 UI-M-AQ1-adz-b-02-0-UI s1 Mus musculus cDNA, 3 end/clone = UI-M-AQ1-adz-b-02-0-UI/ | 220.3 | M | 463 | P | 287 | P | 366.2 |
| | | clone_end = 3/gb = AI843335/gi = 5477548/ug = Mm.27895/len = 197/STRA = for | | | | | | | |
| 20 | 95599_at | Cluster Incl D28941 Mouse mRNA for alpha-2,3-sialyltransferase, complete cds/cds = (128,1129)/gb = D28941/gi = 469215/ | 238.3 | P | 199.4 | M | 219.9 | P | 336.5 |
| | | ug = Mm 2793/len = 1919/STRA = for | | | | | | | |
| 20 | 96208_at | Cluster Incl AW123514.UI-M-BH2 1-aqb-c-04-0-UI s1 Mus musculus cDNA, 3 end/clone = UI-M-BH2 1-aqb-c-04-0-UI/ | 182.7 | P | 208.6 | P | 161.8 | P | 241.6 |
| | | clone_end = 3/gb = AW123514/gi = 6099044/ug = Mm 28646/len = 374/STRA = for | | | | | | | |
| 20 | 96824_at | Cluster Incl AB025354: Mus musculus genes for Supl15h and Sox15.partial cds/cds = (0,168)/gb = AB025354/gi = 5103143/ | 326.8 | P | 639.9 | P | 473.6 | A | 605.9 |
| | | ug = Mm 29387/len = 169/STRA = for | | | | | | | |
| 20 | 97354_at | Cluster Incl AI836082: UI-M-AJ0-abb-f-05-0-UI s1 Mus musculus cDNA, 3 end/clone = UI-M-AJ0-abb-f-05-0-UI/ | 245.2 | P | 242.9 | A | 226.1 | P | 337.5 |
| | | clone_end = 3/gb = AI836082/gi = 5470295/ug = Mm 29630/len = 432/STRA = rev | | | | | | | |
| 20 | 99489_at | Cluster Incl U23921 Mus musculus osmotic stress protein 94 (Osp94) mRNA, complete cds/cds = (182,2698)/gb = U23921/ | 227.3 | P | 131.6 | M | 99 | P | 227.8 |
| | | gi = 1098540/ug = Mm 4150/len = 2898/STRA = for | | | | | | | |
| 20 | 99952_at | Cluster Incl X93035 Glycoprotein 39/cds = (65,1210)/gb = X93035/gi = 1085065/ug = Mm 4376/len = 1616/STRA = rev | 36.5 | A | 130.5 | P | 96 | A | 1388.7 |
| 20 | 100882_at | Cluster Incl AF003525 Defensin beta 1/cds = (42,251)/gb = AF003525/gi = 2197074/ug = Mm.5341/len = 398/STRA = for | 156.8 | P | 134.4 | P | 173.8 | P | 743.4 |
| 20 | 103222_at | Cluster Incl L21671 Mouse Eps8 mRNA sequence, complete cds/cds = (245,2710)/gb = L21671/gi = 309216/ug = Mm 2012/ | 100.1 | P | 36.5 | P | 56.7 | P | 94.4 |
| | | len = 3550/STRA = rev | | | | | | | |
| 20 | 104173_at | Cluster Incl AA797989 ub60d04.r1 Mus musculus cDNA, 5 end/clone = IMAGE-1382119/clone_end = 5/gb = AA797989/ | 464.3 | P | 391.5 | P | 352.6 | P | 545.7 |
| | | gi = 2860944/ug = Mm 27253/len = 413/STRA = for | | | | | | | |
| 20 | 104211_at | Cluster Incl D13867 Mouse mRNA for VLA-3 alpha subunit/cds = (0, 3161)/gb = D13867/gi = 220634/ug = Mm 2775/len = 3162/ | 431 | P | 416.9 | P | 367.1 | P | 463.6 |
| | | STRA = for | | | | | | | |
| 20 | 92979_at | Cluster Incl X63190 Polyomavirus enhancer activator 3/cds = (125, 1792)/gb = X63190/gi = 53627/ug = Mm 5025/len = 2410/ | 182.4 | P | 149.6 | M | 214.2 | P | 253.7 |
| | | STRA = for | | | | | | | |
| 20 | 93672_at | Cluster Incl U09928 Protein kinase, interferon inducible double stranded RNA dependent/cds = (16,1563)/gb = U09928/ | 143.3 | P | 89.6 | A | 84.6 | P | 136.4 |
| | | gi = 3273413/ug = Mm 807/len = 2191/STRA = for | | | | | | | |
| 20 | 93914_at | Cluster Incl M20658 Interleukin 1 receptor, type 1/cds = (224,1954)/gb = M20658/gi = 198300/ug = Mm 896/len = 4740/ | 285.8 | P | 318.4 | P | 363.7 | P | 402 |
| | | STRA = for | | | | | | | |
| 20 | 94753_at | Clusler Incl M80632 Guanine nucleotide binding protein, alpha 15/cds = (42,1166)/gb = M80632/gi = 193570/ug = Mm 1546/ | 196 | P | 135.9 | P | 17.1 | A | 201.4 |
| | | len = 1353/STRA = for | | | | | | | |
| 20 | 100353_g_at | Cluster Incl AA919208 vy50h03 r1 Mus musculus cDNA, 5 end/clone = IMAGE-1298933/clone_end = 5/gb = AA919208/ | 296.3 | P | 250.7 | A | 208.5 | P | 275.3 |
| | | gi = 3065987/ug = Mm 88724/len = 501/STRA = for | | | | | | | |
| 20 | 95794_f_at | Cluster Incl AJ005567 Small proline-rich protein 21/cds = (0,230)/gb = AJ005567/gi = 3093372/ug = Mm 87821/len = 231/ | 552.3 | P | 621.2 | P | 416.3 | A | 1782.9 |
| | | STRA = for | | | | | | | |
| 20 | 93602_at | Cluster Incl AF074714 Mus musculus milogen-and stress-activated protein kinase-2 (mMSK2) mRNA, complete cds/ | 467.5 | P | 516.9 | P | 423.9 | P | 1538 |
| | | cds = (52,2373)/gb = AF074714/gi = 3786405/ug = Mm 20914/len = 3120/STRA = for | | | | | | | |
| 20 | 96259_at | Cluster Incl U35646 Puromycin-sensitive aminopeptidase/cds = (105,2867)/gb = U35646/gi = 1184160/ug = Mm.29824/ | 610.6 | P | 450.2 | A | 404.8 | A | 669.2 |
| | | len = 3051/STRA = for | | | | | | | |
| 20 | 100621_at | Cluster Incl AI848825 UI-M-AJ1-ahb-b-03-0-UI s1 Mus musculus cDNA, 3 end/clone = UI-M-AJ1-ahb-b-03-0-UI/ | 221.4 | A | 251.1 | P | 395.8 | P | 1581.4 |
| | | clone_end = 3/gb = AI848825/gi = 5492731/ug = Mm.6588/len = 342/STRA = for | | | | | | | |
| 20 | 104136_at | Cluster Incl AI840413 UI-M-AH0-acv-h-08-0-UI.s1 Mus musculus cDNA, 3 end/clone = UI-M-AH0-acv-h-08-0-UI/ | 209.7 | P | 184.7 | A | 201.1 | P | 198.5 |
| | | clone_end = 3/gb = AI840413/gi = 5474626/ug = Mm.22094/len = 266/STRA = rev | | | | | | | |
| 20 | 104374_at | Cluster Incl M64086 Mouse spi2 proteinase inhibitor (spi2/eb4) mRNA, complete cds/cds = (60,1316)/gb = M64086/ | 220.9 | M | 150.1 | P | 110.3 | P | 1661.3 |
| | | gi = 201039/ug = Mm.22650/len = 2035/STRA = rev | | | | | | | |
| 20 | 104737_at | Cluster Incl AA689670 vs03b05.r1 Mus musculus cDNA, 5 end/clone = IMAGE-1137105/clone_end = 5/gb = AA669670/ | 169.6 | A | 203.5 | P | 273.6 | P | 672.7 |
| | | gi = 2690606/ug = Mm.24660/len = 238/STRA = for | | | | | | | |
| 20 | 95034_f_at | Cluster Incl AW212243 uo84d09 x1 Mus musculus cDNA, 3 end/clone = IMAGE-2649233/clone_end = 3/gb = AW212243/ | 232 | P | 234 | P | 172.1 | P | 327.6 |
| | | gi = 6518272/ug = Mm 27546/len = 250/STRA = for | | | | | | | |
| 20 | 95618_at | Clusler Incl AI843884 UI-M-AK1-aeu-e-10-0-UI s1 Mus musculus cDNA, 3 end/clone = UI-M-AK1-aeu-e-10-0-UI/ | 171.5 | A | 181.6 | P | 158.6 | P | 230.2 |
| | | clone_end = 3/gb = AI843884/gi = 5478097/ug = Mm.28032/len = 392/STRA = rev | | | | | | | |
| 20 | 103238_at | Cluster Incl M89797 Wingless-related MMTV integration site 4/cds = (45,1100)/gb = M89797/gi = 202401/ug = Mm 20355/ | 311.5 | P | 309.9 | P | 418.6 | P | 269.4 |
| | | len = 1101/STRA = for | | | | | | | |
| 20 | 103445_at | Cluster Incl M18401 Mus musculus putative homeobox 2.2 protein gene, partial cds/cds = (0,293)/gb = M18401/gi = 193938/ | 355.4 | P | 183.1 | P | 237.8 | A | 319.4 |
| | | ug = Mm.215/len = 294/STRA = for | | | | | | | |
| 20 | 104198_at | Cluster Incl AW211760 uo85c06 y1 Mus musculus cDNA, 5 end/clone = IMAGE-2649322/clone_end = 5/gb = AW211760/ | 374.4 | P | 313.4 | M | 339.1 | P | 452.2 |
| | | gi = 6517732/ug = Mm 27519/len = 462/STRA = for | | | | | | | |
| 20 | 104210_at | Cluster Incl AI839082 UI-M-AK0-adl-f-03-0-UI s1 Mus musculus cDNA, 3 end/clone = UI-M-AK0-adl-f-03-0-UI/clone_end = 3/ | 191.6 | P | 44.3 | A | 155.8 | M | 158.6 |
| | | gb = AI839082/gi = 5473295/ug = Mm.2775/len = 212/STRA = for | | | | | | | |
| 20 | 92276_at | Cluster Incl AB021861: Mus musculus mRNA for apoptosis signal-regulating kinase 2, complete cds/cds = (269,4138)/ | 333.4 | P | 181.8 | A | 289.9 | P | 288.2 |
| | | gb = AB021861/gi = 4996560/ug = Mm.36640/len = 4325/STRA = for | | | | | | | |
| 20 | 92280_at | Cluster Incl AA867778 vx16e04 r1 Mus musculus cDNA, 5 end/clone = IMAGE-1264638/clone_end = 5/gb = AA867778/ | 185 | P | 149.8 | A | 219 | P | 236.3 |
| | | gi = 2963223/ug = Mm 3678/len = 512/STRA = for | | | | | | | |
| 20 | 92759_at | Cluster Incl U43298 Laminin, beta 3/cds = (156,3662)/gb = U43298/gi = 1151214/ug = Mm 4732/len = 4049/STRA = for | 93.3 | A | 278.9 | A | 364.6 | P | 533.3 |
| 20 | 94791_s_at | Cluster Incl M90470 Leukocyte tyrosine kinase/cds = (137,2803)/gb = M90470/gi = 198885/ug = Mm.1740/len = 3077/ | 430.7 | P | 167.9 | M | 243.6 | M | 851.2 |
| | | STRA = for | | | | | | | |
| 20 | 96566_at | Cluster Incl L10385.Mus musculus transglutaminase E (TGASE3) mRNA, complete cds/cds = (54,2135)/gb = L10385/ | 173 | P | 242.1 | P | 230.6 | A | 337.5 |
| | | gi = 309520/ug = Mm 2961/len = 2297/STRA = for | | | | | | | |
| 20 | 98372_at | Cluster Incl AW050387 UI-M-BH1-ang-h-10-0-UI s1 Mus musculus cDNA, 3 end/clone = UI-M-BH1-ang-h-10-0-UI/ | 129.1 | P | 149.5 | A | 47.3 | A | 131.4 |
| | | clone_end = 3/gb = AW050387/gi = 5910916/ug = Mm.4458/len = 380/STRA = for | | | | | | | |
| 20 | 95793_at | Cluster Incl AJ005562 Small proline-rich protein 2D/cds = (43,300)/gb = AJ005562/gi = 3093362/ug = Mm 87820/len = 668/ | 114.8 | A | 119.5 | A | 147.7 | P | 2804.9 |
| | | STRA = for | | | | | | | |
| 20 | 92870_at | Cluster Incl AF063095.Mus musculus SEL1L (Sel1l) mRNA, complete cds/cds = (75,2297)/gb = AF063095/gi = 4159994/ | 212.3 | P | 147.6 | A | 167.8 | P | 251 |
| | | ug = Mm 14352/len = 3663/STRA = for | | | | | | | |
| 20 | 93105_s_at | Cluster Incl M26056 Mouse T-cell receptor germline beta-chain gene constant region (CT)/cds = (0,520)/gb = M26056/ | 274.4 | A | 300.2 | A | 281.5 | P | 369 |
| | | gi = 201239/ug = Mm 16654/len = 521/STRA = for | | | | | | | |
| 20 | 93795_at | Cluster Incl AW124626 UI-M-BH2 1-apw-b-07-0-UI.s1 Mus musculus cDNA, 3 end/clone = UI-M-BH2 1-apw-b-07-0-UI/ | 229.3 | P | 272.1 | P | 221.9 | P | 309.4 |
| | | clone_end = 3/gb = AW124626/gi = 6100156/ug = Mm 21399/len = 416/STRA = for | | | | | | | |
| 20 | 94260_at | Cluster Incl AI850352 UI-M-BG1-aig-a-08-0-UI s1 Mus musculus cDNA, 3 end/clone = UI-M-BG1-aig-a-08-0-UI/ | 456.5 | A | 425.6 | P | 395.6 | P | 755.2 |
| | | clone_end = 3/gb = AI850352/gi = 5494258/ug = Mm 22428/len = 420/STRA = for | | | | | | | |
| 20 | 94316_at | Cluster Incl AB012042 Mus musculus gene for keratin 6 beta/cds = (287,1975)/gb = AB012042/gi = 3845567/ug = Mm 22657/ | 527 | P | 1059.9 | P | 433.6 | P | 2125.3 |
| | | len = 2299/STRA = for | | | | | | | |
| 20 | 104601_at | Cluster Incl X14432 Thrombomodulin/cds = (158,1891)/gb = X14432/gi = 54781/ug = Mm 24096/len = 3636/STRA = for | 74.8 | A | 88.9 | A | 129.8 | P | 263.9 |
| 20 | 97420_at | Cluster Incl AW230891 uo69b04.y1 Mus musculus cDNA, 5 end/clone = IMAGE-2647759/clone_end = 5/gb = AW230891/ | −4.2 | A | 53.5 | P | 77.9 | P | 1193.2 |
| | | gi = 6560187/ug = Mm 29986/len = 523/STRA = rev | | | | | | | |
| 20 | 100884_at | Cluster Incl U04204 Fibroblast growth factor regulated protein/cds = (67,1017)/gb = U04204/gi = 463376/ug = Mm 5378/ | 77.3 | M | 181.4 | P | 165.2 | P | 550.5 |
| | | len = 1304/STRA = for | | | | | | | |
| 20 | 101464_at | Cluster Incl V00755 Interferon beta, fibroblast/cds = (0,118)/gb = V00755/gi = 51554/ug = Mm 8245/len = 222/STRA = for | 1063.7 | A | 1007.1 | A | 1122.7 | P | 2521.2 |
| 20 | 102889_r_at | Cluster Incl X86569 LIM-domain containing, protein kinase/cds = (322,2265)/gb = X86569/gi = 1051159/ug = Mm.15409/ | 413.8 | P | 260.1 | A | 286.9 | A | 503.8 |
| | | len = 3439/STRA = for | | | | | | | |
| 20 | 93000_g_at | Cluster Incl D50416 Mouse mRNA for AREC3, complete cds/cds = (61,2388)/gb = D50416/gi = 1255625/ug = Mm.5085/ | 110 | A | 67.5 | P | 110.5 | A | 208.5 |
| | | len = 5566/STRA = for | | | | | | | |
| 20 | 93204_r_at | Cluster Incl AW107463 um17a12 x1 Mus musculus cDNA, 3 end/clone = IMAGE-2192542/clone_end = 3/gb = AW107463/ | 23.6 | A | 38.6 | P | 37.3 | P | 41.4 |
| | | gi = 6078263/ug = Mm.5697/len = 484/STRA = rev | | | | | | | |
| 20 | 97790_s_at | Cluster Incl X84014 Laminin, alpha 3/cds = (0,7710)/gb = X84014/gi = 1922890/ug = Mm.42012/len = 8077/STRA = for | −47.2 | A | −60.6 | A | 15 | P | 123.4 |
| 20 | 100333_at | Cluster Incl M13521 Murine serum amyloid A-1 (SAA-1) gene/cds = (0,368)/gb = M13521/gi = 200905/ug = Mm 87850/ | 517 | P | 528.4 | P | 391.4 | A | 899.2 |
| | | len = 369/STRA = for | | | | | | | |
| 20 | 92858_at | Cluster Incl AF002719 Mus musculus secretory leukoprotease inhibitor gene, complete cds/cds = (20,415)/gb = AF002719/ | 6.4 | A | −2.3 | P | 80 | A | 1402.8 |
| | | gi = 4100898/ug = Mm 1395/len = 660/STRA = for | | | | | | | |
| 20 | 93603_at | Cluster Incl AF034092 Mus musculus nuclear localization signal containing protein (Nlvcf) mRNA, complete cds/ | 231.6 | P | 179.6 | P | 107.6 | A | 299.8 |
| | | cds = (114,734)/gb = AF034092/gi = 3834385/ug = Mm.20918/len = 874/STRA = for | | | | | | | |
| 20 | 96093_at | Cluster Incl AI842705 UI-M-AO1-aeo-d-09-0-UI s1 Mus musculus cDNA, 3 end/clone = UI-M-AO1-aeo-d-09-0-UI/ | 445.8 | P | 294.6 | P | 343.5 | P | 439.1 |
| | | clone_end = 3/gb = AI842705/gi = 5476918/ug = Mm.26740/len = 300/STRA = for | | | | | | | |
| 20 | 98892_at | Cluster Incl AI846934 UI-M-AK1-aey-e-06-0-UI s1 Mus musculus cDNA, 3 end/clone = UI-M-AK1-aey-e-06-0-UI/ | 36.8 | P | 40.3 | P | 53.3 | M | 63.1 |
| | | clone_end = 3/gb = AI846934/gi = 5490840/ug = Mm.28548/len = 407/STRA = for | | | | | | | |
| 20 | 104730_at | Cluster Incl AW046889: UI-M-BH1-akl-d-04-0-UI s1 Mus musculus cDNA, 3 end/clone = UI-M-BH1-akl-d-04-0-UI/ | 264.8 | P | 107.5 | P | 96.7 | P | 120.9 |
| | | clone_end = 3/gb = AW046889/gi = 5907418/ug = Mm 24627/len = 369/STRA = rev | | | | | | | |
| 20 | 94383_at | Cluster Incl D50586 Tissue factor pathway inhibitor 2/cds = (35,727)/gb = D50586/gi = 2467368/ug = Mm 25612/len = 1522/ | 30.3 | A | 18.9 | A | 39.5 | P | 111.2 |
| | | STRA = for | | | | | | | |
| 20 | 94924_at | Cluster Incl AW122679 UI-M-BH2 2-aol-h-11-0-UI s1 Mus musculus cDNA, 3 end/clone = UI-M-BH2.2-aol-h-11-0-UI/ | 699.7 | P | 636.5 | P | 577.1 | M | 844.1 |
| | | clone_end = 3/gb = AW122679/gi = 6098209/ug = Mm 2664/len = 291/STRA = for | | | | | | | |
| 20 | 96764_at | Cluster Incl AJ007971 Mus musculus mRNA for IIGP protein/cds = (104,1345)/gb = AJ007971/gi = 4158173/ug = Mm 29008/ | 261.1 | P | −4.5 | A | −11 | A | 790.7 |
| | | len = 2280/STRA = for | | | | | | | |
| 20 | 100976_at | Cluster Incl AF013490 Mus musculus protein-tyrosine phosphatase mRNA, complete cds/cds = (96,1877)/gb = AF013490/ | 500.2 | A | 392.8 | M | 277.2 | P | 450.5 |
| | | gi = 2331298/ug = Mm.6678/len = 3531/STRA = for | | | | | | | |
| 20 | 101384_at | Cluster Incl AI852933 UI-M-BH0-aiz-c-09-0-UI s1 Mus musculus cDNA, 3 end/clone = UI-M-BH0-aiz-c-09-0-UI/ | 152.6 | A | 161.8 | A | 162 | A | 536.5 |
| | | clone_end = 3/gb = AI852933/gi = 5496839/ug = Mm.7114/len = 395/STRA = for | | | | | | | |
| 20 | 102689_at | Cluster Incl AF110520 Mus musculus major histocompatibility complex region NG27, NG28, RPS28, NADH | 305.6 | A | 343.1 | M | 300.7 | P | 369.2 |
| | | oxidoreductase, NG29, KIFC1, Fas-binding protein, BING1, tapasin, RalGDS-like, KE2, BING4, beta 1,3-galactosyl | | | | | | | |
| | | transferase, and RPS18 genes, complete cds, S/STRA = for | | | | | | | |
| 20 | 102712_at | Cluster Incl X03505 Serum amyloid A 3/cds = (0,368)/gb = X03505/gi = 54028/ug = Mm 14277/len = 369/STRA = for | 82.8 | P | 30.8 | M | 5.8 | A | 2206.1 |
| 20 | 104035_at | Cluster Incl AW123795 UI-M-BH2.1-apm-e-02-0-UI s1 Mus musculus cDNA, 3 end/clone = UI-M-BH2 1-apm-e-02-0-UI/ | 91.9 | A | 10.4 | A | 7.4 | A | 157.1 |
| | | clone_end = 3/gb = AW123795/gi = 6099325/ug = Mm 27057/len = 404/STRA = rev | | | | | | | |
| 20 | 93145_at | Cluster Incl AI840824 UI-M-AH0-adb-d-03-0-UI s1 Mus musculus cDNA, 3 end/clone = UI-M-AH0-adb-d-03-0-UI/ | 79.8 | A | 85.7 | A | 74.4 | P | 129.4 |
| | | clone_end = 3/gb = AI840824/gi = 5475037/ug = Mm 5209/len = 447/STRA = for | | | | | | | |
| 20 | 93878_at | Cluster Incl AF010135 Mus musculus maf10 (mAF10) mRNA, complete cds/cds = (178,3384)/gb = AF010135/gi = 2809380/ | 182.8 | A | 211.9 | A | 194.7 | A | 239.5 |
| | | ug = Mm 8866/len = 3543/STRA = for | | | | | | | |
| 20 | 100308_at | Cluster Incl X66976.M. musculus col8a1 gene, exon 1 (and joined CDS)/cds = (0,2231)/gb = X66976/gi = 50493/ | 177.3 | P | 120.3 | M | 111.7 | P | 171.5 |
| | | ug = Mm 86813/len = 2232/STRA = for | | | | | | | |
| 20 | 97598_at | Cluster Incl AW209139: uo02d07.x1 Mus musculus cDNA, 3 end/clone = IMAGE-2582221/clone_end = 3/gb = AW209139/ | 15.9 | P | 32.1 | A | 17.7 | P | 143.1 |
| | | gi = 6515079/ug = Mm.89650/len = 425/STRA = rev | | | | | | | |
| 20 | 102423_at | Cluster Incl AI649125 uk31e12 x1 Mus musculus cDNA, 3 end/clone = IMAGE-1970638/clone_end = 3/gb = AI649125/ | 50.4 | A | 25 | A | 22.6 | A | 71.1 |
| | | gi = 4729959/ug = Mm.12790/len = 593/STRA = for | | | | | | | |
| 20 | 102763_at | Cluster Incl AF064748 Mus musculus S3-12 mRNA, complete cds/cds = (138,4349)/gb = AF064748/gi = 3236367/ | 42.9 | P | 125.6 | A | 115.5 | P | 216.9 |
| | | ug = Mm 12966/len = 5774/STRA = for | | | | | | | |
| 20 | 103080_at | Cluster Incl U15635 Mus musculus IFN-gamma induced (Mg11) mRNA, complete cds/cds = (57,1841)/gb = U15635/ | 79.4 | A | 139.3 | A | 122.6 | A | 440 |
| | | gi = 558902/ug = Mm.17167/len = 1942/STRA = for | | | | | | | |
| 20 | 103634_at | Cluster Incl U51992.lnterferon dependent positive acting transcription factor 3 gamma/cds = (64,1263)/gb = U51992/ | 152 | A | 118.3 | A | −1.4 | A | 409.1 |
| | | gi = 1263309/ug = Mm 2032/len = 1959/STRA = for | | | | | | | |
| 20 | 104293_at | Cluster Incl AI882440 uc01f05 r1 Mus musculus cDNA, 5 end/clone = IMAGE-1396737/clone_end = 5/gb = AI882440/ | −151.7 | A | −203.6 | A | −71.6 | P | −83.2 |
| | | gi = 5567529/ug = Mm 22306/len = 271/STRA = for | | | | | | | |
| 20 | 96123_at | Cluster Incl X99347 M musculus mRNA for LPS-binding protein/cds = (127,1572)/gb = X99347/gi = 1430866/ug = Mm 28110/ | 38.3 | A | 171.8 | P | 77.5 | A | 262 |
| | | len = 1925/STRA = for | | | | | | | |
| 20 | 96766_s_at | Cluster Incl AB000828 Mouse mRNA for receptor tyrosine kinase, complete cds/cds = (13,2655)/gb = AB000828/ | 90 | A | 170.9 | A | 198.2 | P | 218.6 |
| | | gi = 1871412/ug = Mm 2901/len = 2845/STRA = for | | | | | | | |
| 20 | 97971_at | Cluster Incl AI852148 UI-M-BH0-ajb-d-12-0-UI s1 Mus musculus cDNA, 3 end/clone = UI-M-BH0-ajb-d-12-0-UI/ | 156.6 | A | 206.1 | P | 173.3 | A | 241.1 |
| | | clone_end = 3/gb = AI852148/gi = 5496049/ug = Mm 30991/len = 375/STRA = for | | | | | | | |
| 20 | 98501_at | Cluster Incl Y07519 Lymphocyte antigen 84/cds = (194,1207)/gb = Y07519/gi = 55517/ug = Mm 35692/len = 2646/STRA = for | 8.7 | A | −15.7 | A | −27.2 | A | 629 |
| 20 | 100484_at | Cluster Incl X66473 Matrix metalloproteinase 1/cds = (10,1428)/gb = X66473/gi = 53603/ug = Mm 5022/len = 2660/STRA = for | 30 | A | 42.6 | A | 39.7 | A | 117.7 |
| 20 | 100983_at | Cluster Incl AB022053 Mus musculus gene for prolyl oligopeptidase/cds = (377,2509)/gb = AB022053/gi = 5689034/ | 225.7 | M | 223.7 | M | 173.6 | P | 332.3 |
| | | ug = Mm 6735/len = 2981/STRA = for | | | | | | | |
| 20 | 102637_at | Cluster Incl AF039601 Mus musculus betaglycan mRNA, complete cds/cds = (231,2783)/gb = AF039601/gi = 3387819/ | 42.6 | A | 32.6 | A | 79.4 | P | 94.7 |
| | | ug = Mm 12960/len = 3977/STRA = rev | | | | | | | |
| 20 | 103446_at | Cluster Incl AA959954 vw53a09 s1 Mus musculus cDNA, 3 end/clone = IMAGE-1247512/clone_end = 3/gb = AA959954/ | −36.8 | A | −9 | P | −166.5 | A | 124.2 |
| | | gi = 3125854/ug = Mm 21551/len = 524/STRA = for | | | | | | | |
| 20 | 92234_at | Cluster Incl X66223 M musculus mRNA for retinoid X receptor-alpha (mRXR-alpha)/cds = (177,1580)/gb = X66223/ | 336.6 | A | 71.6 | A | 267.8 | A | 475.2 |
| | | gi = 54021/ug = Mm 3470/len = 2088/STRA = for | | | | | | | |
| 20 | 92353_at | Cluster Incl Y18723 Mus musculus mRNA for secreted senne protease (BSSP)/cds = (237,977)/gb = Y18723/gi = 4220545/ | 207 | A | 118 | A | −205.8 | A | 1733.9 |
| | | ug = Mm 3944/len = 1122/STRA = rev | | | | | | | |
| 20 | 93866_s_at | Cluster Incl D00613 Matrix gamma-carboxyglutamate (gia) protein/cds = (18,332)/gb = D00613/gi = 220478/ug = Mm 87793/ | 99.4 | P | 108.5 | A | 132.6 | M | 212.8 |
| | | len = 540/STRA = for | | | | | | | |
| 20 | 94120_s_at | Cluster Incl AJ005564 Small proline-nch protein 2F/cds = (23,253)/gb = AJ005564/gi = 3093366/ug = Mm 10692/len = 570/ | 146.6 | M | 108.5 | A | 100.7 | M | 2370.1 |
| | | STRA = for | | | | | | | |
| 20 | 98772_at | Cluster Incl U27267.Small inducible cytokine B subfamily, member 5/cds = (65,463)/gb = U27267/gi = 950158/ug = Mm 4660/ | 12.4 | A | 42.4 | A | 71.5 | A | 532.7 |
| | | len = 1519/STRA = for | | | | | | | |
| 20 | 98833_at | Cluster Incl X66402 Matrix metalloproteinase 3/cds = (24,1457)/gb = X66402/gi = 296167/ug = Mm.4993/len = 1740/ | 68 | A | 72.2 | A | 30.4 | A | 510.8 |
| | | STRA = for | | | | | | | |

| | Affymetrix | | | | | PU.1 | | | |
|---|---|---|---|---|---|---|---|---|---|
| | accession | | | WT 24 | | KO | | PU.1 KO | |
| Cluster | number | Description | Call | hours | Call | control | Call | 30 min | Call |
| 1 | 92769_at | Cluster Incl M25149: Tissue specific transplantation antigen P91A/cds = (0,1589)/gb = M25149/gi = 191974/ug = Mm 12194/ | P | 731.8 | P | 781 | P | 937. 7 | P |
| | | len = 1590/STRA = for | | | | | | | |
| 1 | 93565_at | Cluster Incl AF055665: Kinesin light chain 1/cds = (94,1719)/gb = AF055665/gi = 3347845/ug = Mm 20354/len = 2289/ | P | 204.5 | P | 223.5 | P | 322.2 | P |
| | | STRA = for | | | | | | | |
| 1 | 93589_at | Cluster Incl AI851172: UI-M-BH0-akg-g-03-0-UI s1 Mus musculus cDNA, 3 end/clone = UI-M-BH0-akg-g-03-0-UI/ | P | 732.3 | P | 735.6 | P | 1024 | P |
| | | clone_end = 3/gb = AIB51172/gi = 5495043/ug = Mm 20806/len = 363/STRA = for | | | | | | | |
| 1 | 93749_at | Cluster Incl AI848045: UI-M-AH1-agp-g-05-0-UI s1 Mus musculus cDNA, 3 end/clone = UI-M-AH1-agp-g-05-0-UI/ | P | 206 | P | 129.6 | P | 174.9 | P |
| | | clone_end = 3/gb = AI848045/gi = 5491951/ug = Mm 21108/len = 224/STRA = for | | | | | | | |
| 1 | 93757_at | Cluster Incl AI843574 UI-M-AO1-aen-d-12-0-UI s1 Mus musculus cDNA, 3 end/clone = UI-M-AO1-aen-d-12-0-UI/ | P | 1292.9 | P | 1200.9 | P | 1593.8 | P |
| | | clone_end = 3/gb = AI843574/gi = 5477827/ug = Mm 21132/len = 352/STRA = for | | | | | | | |
| 1 | 94024_at | Cluster Incl AA671429 vn81g12 r1 Mus musculus cDNA, 5 end/clone = IMAGE-1038406/clone_end = 5/gb = AA671429/ | P | 723.7 | P | 909.5 | P | 1311.9 | P |
| | | gi = 2643508/ug = Mm 21873/len = 464/STRA = for | | | | | | | |
| 1 | 94254_at | Cluster Incl AI845237 UI-M-BG0-aht-g-09-0-UI s1 Mus musculus cDNA, 3 end/clone = UI-M-BG0-aht-g-09-0-UI/ | P | 914.6 | P | 1060.3 | P | 1048.2 | P |
| | | clone_end = 3/gb = AI845237/gi = 5489143/ug = Mm 22409/len = 255/STRA = for | | | | | | | |
| 1 | 94898_at | Cluster Incl AI848555 UI-M-AP1-agf-h-08-0-UI.s2 Mus musculus cDNA, 3 end/clone = UI-M-AP1-agf-h-08-0-UI/ | P | 332.5 | P | 212.4 | P | 353 | P |
| | | clone_end = 3/gb = AI848555/gi = 5492461/ug = Mm 24015/len = 426/STRA = for | | | | | | | |
| 1 | 96001_at | Cluster Incl AW122293 UI-M-BH2 2-aos-c-01-0-UI s1 Mus musculus cDNA, 3 end/clone = UI-M-BH2 2-aos-c-01-0-UI/ | P | 893.9 | P | 1167.8 | P | 1256.6 | P |
| | | clone_end = 3/gb = AW122293/gi = 6097778/ug = Mm 25321/len = 363/STRA = for | | | | | | | |
| 1 | 96045_at | Cluster Incl AW050142 UI-M-BH1-ano-e-11-0-UI.s1 Mus musculus cDNA, 3 end/clone = UI-M-BH1-ano-e-11-0-UI/ | P | 177.5 | P | 235.9 | P | 350.1 | P |
| | | clone_end = 3/gb = AW050142/gi = 5910671/ug = Mm 25994/len = 458/STRA = for | | | | | | | |
| 1 | 96672_at | Cluster Incl AW123564 UI-M-BH2.1-aqb-h-01-0-UI.s1 Mus musculus cDNA, 3 end/clone = UI-M-BH2.1-aqb-h-01-0-UI/ | P | 1941.3 | P | 1857.6 | P | 2605.4 | P |
| | | clone_end = 3/gb = AW123564/gi = 6099094/ug = Mm 27401/len = 365/STRA = for | | | | | | | |
| 1 | 96716_at | Cluster Incl AW121102: UI-M-BH2 3-aoh-b-01-0-UI s1 Mus musculus cDNA, 3 end/clone = UI-M-BH2 3-aoh-b-01-0-UI/ | P | 521.7 | P | 553.7 | P | 583.2 | P |
| | | clone_end = 3/gb = AW121102/gi = 6096435/ug = Mm 27647/len = 353/STRA = rev | | | | | | | |
| 1 | 97226_at | Cluster Incl AW125649: UI-M-BH2.2-aqi-b-03-0-UI s1 Mus musculus cDNA, 3 end/clone = UI-M-BH2 2-aqi-b-03-0-UI/ | P | 299.3 | P | 325.1 | P | 400.1 | P |
| | | clone_end = 3/gb = AW125649/gi = 6101179/ug = Mm 27773/len = 435/STRA = for | | | | | | | |
| 1 | 97263_s_at | Clusler Incl AI846289 UI-M-AK1-aez-f-02-0-UI s1 Mus musculus cDNA, 3 end/clone = UI-M-AK1-aez-f-02-0-UI/ | P | 856.4 | P | 978.6 | P | 1401.8 | P |
| | | clone_end = 3/gb = AI846289/gi = 5490195/ug = Mm 27899/len = 375/STRA = for | | | | | | | |
| 1 | 97284_at | Clusler Incl AI853789 UI-M-BH0-aig-f-10-0-UI s1 Mus musculus cDNA, 3 end/clone = UI-M-BH0-aig-f-10-0-UI/ | P | 633.8 | P | 621.6 | P | 690.5 | P |
| | | clone_end = 3/gb = AI853789/gi = 5497695/ug = Mm 27990/len = 458/STRA = for | | | | | | | |
| 1 | 97817_at | Cluster Incl AW121136 UI-M-BH2 3-aoh-e-05-0-UI s1 Mus musculus cDNA, 3 end/clone = UI-M-BH2 3-aoh-e-05-0-UI/ | P | 985.7 | P | 796.4 | P | 995.6 | P |
| | | clone_end = 3/gb = AW121136/gi = 6096469/ug = Mm 28189/len = 391/STRA = for | | | | | | | |
| 1 | 97823_g_at | Cluster Incl AW122689 UI-M-BH2 2-aot-a-10-0-UI s1 Mus musculus cDNA, 3 end/clone = UI-M-BH2 2-aot-a-10-0-UI/ | P | 702.1 | P | 719.3 | P | 810.4 | P |
| | | clone_end = 3/gb = AW122689/gi = 6098219/ug = Mm 28202/len = 467/STRA = rev | | | | | | | |
| 1 | 97871_at | Cluster Incl AW120614 UI-M-BH2 3-anx-g-02-0-UI s1 Mus musculus cDNA, 3 end/clone = UI-M-BH2 3-anx-g-02-0-UI/ | P | 436 | P | 393.6 | P | 540.9 | P |
| | | clone_end = 3/gb = AW120614/gi = 6095947/ug = Mm.28339/len = 294/STRA = rev | | | | | | | |
| 1 | 98882_s_at | Cluster Incl AW047320 UI-M-BH1-amh-g-06-0-UI s1 Mus musculus cDNA, 3 end/clone = UI-M-BH1-amh-g-06-0-UI/ | P | 1329.1 | P | 1247.2 | P | 1714.7 | P |
| | | clone_end = 3/gb = AW047320/gi = 5907849/ug = Mm.28527/len = 616/STRA = for | | | | | | | |
| 1 | 94451_at | Cluster Incl AI787627 UI20f06 y1 Mus musculus cDNA, 5 end/clone = IMAGE-2088131/clone_end = 5/gb = AI787627/ | P | 628.6 | P | 639.7 | P | 697.1 | P |
| | | gi = 5335343/ug = Mm 28786/len = 450/STRA = for | | | | | | | |
| 1 | 95666_at | Cluster Incl AW123404 UI-M-BH2 1-apj-a-09-0-UI s1 Mus musculus cDNA, 3 end/clone = UI-M-BH2 1-apj-a-09-0-UI/ | P | 452.6 | P | 290.7 | P | 501.3 | P |
| | | clone_end = 3/gb = AW123404/gi = 6098934/ug = Mm 29572/len = 354/STRA = for | | | | | | | |
| 1 | 95756_at | Clusler Incl AI839681: UI-M-AN0-acp-d-02-0-UI s1 Mus musculus cDNA, 3 end/clone = UI-M-AN0-acp-d-02-0-UI/ | P | 366.7 | P | 429.5 | P | 518.4 | P |
| | | clone_end = 3/gb = AI839681/gi = 5473894/ug = Mm 29795/len = 348/STRA = for | | | | | | | |
| 1 | 96301_at | Clusler Incl AI851912 UI-M-BH0-aix-e-03-0-UI s1 Mus musculus cDNA, 3 end/clone = UI-M-BH0-aix-e-03-0-UI/ | P | 544.3 | P | 748.1 | P | 1160.6 | P |
| | | clone_end = 3/gb = AI851912/gi = 5495818/ug = Mm 29911/len = 392/STRA = for | | | | | | | |
| 1 | 96357_at | Cluster Incl AW212775 uo66e09.x1 Mus musculus cDNA, 3 end/clone = IMAGE-2647528/clone_end = 3/gb = AW212775/ | P | 494.9 | P | 634.9 | P | 711.4 | P |
| | | gi = 6518896/ug = Mm 30010/len = 165 STRA = for | | | | | | | |
| 1 | 97550_at | Cluster Incl AW047228 UI-M-BH1-amb-f-04-0-UI.s1 Mus musculus cDNA, 3 end/clone = UI-M-BH1-amb-f-04-0-UI/ | P | 640.6 | P | 552.6 | P | 804.7 | P |
| | | clone_end = 3/gb = AW047228/gi = 5907757/ug = Mm.33797/len = 314/STRA = for | | | | | | | |
| 1 | 99559_at | Cluster Incl U14390 Aldehyde dehydrogenase 4, liver microsomal (class 3)/cds = (113,1587)/gb = U14390/gi = 687253/ | P | 851.7 | P | 820.9 | P | 1193.4 | P |
| | | ug = Mm 4210/len = 2997/STRA = for | | | | | | | |
| 1 | 100566_at | Cluster Incl L12447 Insulin-like growth factor binding protein 5/cds = (387,1202)/gb = L12447/gi = 425405/ug = Mm.578/ | P | 513.3 | P | 675.8 | P | 1108.4 | P |
| | | len = 5561/STRA = for | | | | | | | |
| 1 | 101490_at | Cluster Incl AW122692 UI-M-BH2 2-aot-b-01-0-UI s1 Mus musculus cDNA, 3 end/clone = UI-M-BH2 2-aol-b-01-0-UI/ | P | 503.9 | P | 334 | P | 629.4 | P |
| | | clone_end = 3/gb = AW122692/gi = 6098222/ug = Mm 7884/len = 288/STRA = for | | | | | | | |
| 1 | 101520_at | Cluster Incl AF032967 Mus musculus strain C3H/HeN mRNA preferentially expressed In LPS-normoresponsive | P | 283.2 | P | 198.3 | P | 349.3 | P |
| | | macrophages/cds = UNKNOWN/gb = AF032967/gi = 4103990/ug = Mm.8540/len = 1766/STRA = for | | | | | | | |
| 1 | 101579_at | Clusler Incl X78304: M. musculus (SRP9) signal recognition particle subunit mRNA, 689bp/cds = (93,353)/gb = X78304/ | P | 574.2 | P | 431.8 | P | 628.9 | P |
| | | gi = 872130/ug = Mm 89927/len = 689/STRA = for | | | | | | | |
| 1 | 102324_at | Cluster Incl AF036875 Mus musculus multiple endocrine neoplasia type 1 candidate prolein number 18 mRNA, complete | P | 199.7 | P | 136.9 | P | 211.4 | P |
| | | cds/cds = (19,753)/gb = AF036875/gi = 3136115/ug = Mm 10721/len = 841/STRA = for | | | | | | | |
| 1 | 102768_l_at | Cluster Incl AB016248 Mus musculus mRNA for sterol-C5-desaturase, complete cds/cds = (170,1069)/gb = AB016248/ | P | 95.7 | P | 91.6 | P | 116.7 | P |
| | | gi = 3721883/ug = Mm.13081/len = 1721/STRA = for | | | | | | | |
| 1 | 102993_at | Cluster Incl M85153: Glycoprotein galactosyltransferase alpha 1, 3/cds = (444,1559)/gb = M85153/gi = 193563/ug = Mm.1548/ | P | 608.8 | P | 583.4 | P | 731.6 | P |
| | | len = 3450/STRA = for | | | | | | | |
| 1 | 103005_s_at | Cluster Incl X66084 CD44 antigen/cds = (9,1976)/gb = X66084/gi = 53679/ug = Mm 15643/len = 1997/STRA = for | P | 137.3 | P | 190.9 | P | 305.8 | P |
| 1 | 103052_r_at | Cluster Incl X76653 Apolipoprotein regulatory protein 1/cds = (35,1279)/gb = X76653/gi = 482927/ug = Mm.16519/len = 2495/ | P | 146.3 | P | 116.3 | P | 193.2 | P |
| | | STRA = for | | | | | | | |
| 1 | 103345_at | Cluster Incl AW046708: UI-M-BH1-alt-d-05-0-Ul.s1 Mus musculus cDNA, 3 end/clone = UI-M-BH1-alt-d-05-0-UI/ | P | 635.8 | P | 505.5 | P | 812.3 | P |
| | | clone_end = 3/gb = AW046708/gi = 5907277/ug = Mm. 18249/len = 412/STRA = for | | | | | | | |
| 1 | 103669_at | Cluster Incl AA793857: vr40b03.s1 Mus musculus cDNA, 5 end/clone = IMAGE-1123085/clone_end = 5/gb = AA793857/ | P | 373.5 | P | 370.7 | P | 481.1 | P |
| | | gi = 2856812/ug = Mm.2077/len = 261/STRA = for | | | | | | | |
| 1 | 103814_at | Cluster Incl AF093871.Mus musculus peroxisomal biogenesis factor (Pex11b) mRNA, complete cds/cds = (3,782)/ | P | 431.9 | P | 469.3 | P | 613.5 | P |
| | | gb = AF093671/gi = 3907595/ug = Mm 20901/len = 1109/STRA = for | | | | | | | |
| 1 | 103832_at | Cluster Incl AF097181 Mus musculus tuftelin-interacting protein 10 mRNA, complete cds/cds = (96,1676)/gb = AF097181/ | P | 329 | P | 294.3 | P | 450.1 | P |
| | | gi = 3851163/ug = Mm.20933/len = 2424/STRA = for | | | | | | | |
| 1 | 104069_at | Cluster Incl AI852737 UI-M-BH0-aji-h-05-0-UI.s1 Mus musculus cDNA, 3 end/clone = UI-M-BH0-aji-h-05-0-UI/clone_end = 3/ | P | 917.7 | P | 1079.4 | P | 1200.7 | P |
| | | gb = AI852737/gi = 5496643/ug = Mm 21619/len = 503/STRA = rev | | | | | | | |
| 1 | 104091_at | Cluster Incl AW122563 UI-M-BH2 2-aox-e-09-0-UI s1 Mus musculus cDNA, 3 end/clone = UI-M-BH2 2-aox-e-09-0-UI/ | P | 438.6 | P | 637.1 | P | 805.8 | P |
| | | clone_end = 3/gb = AW122563/gi = 6098087/ug = Mm 21814/len = 274/STRA = for | | | | | | | |
| 1 | 104128_at | Cluster Incl AW048075 UI-M-BH1-alq-a-05-0-UI s1 Mus musculus cDNA, 3 end/clone = UI-M-BH1-alq-a-05-0-UI/ | P | 953.8 | P | 1024.2 | P | 1084.7 | P |
| | | clone_end = 3/gb = AW048075/gi = 5908604/ug = Mm 22040/len = 340/STRA = for | | | | | | | |
| 1 | 104574_at | Cluster Incl AW210607.um62e04.x1 Mus musculus cDNA, 3 end/clone = IMAGE-2285982/clone_end = 3/gb = AW210607/ | P | 557.2 | P | 356.8 | P | 597 | P |
| | | gi = 6516547/ug = Mm 23927/len = 418/STRA = rev | | | | | | | |
| 1 | 94973_at | Cluster Incl AA840126 ud01d09 r1 Mus musculus cDNA, 5 end/clone = lMAGE-1433873/clone_end = 5/gb = AA840126/ | P | 218.9 | P | 96.6 | P | 239.4 | P |
| | | gi = 2915835/ug = Mm 27232/len = 471/STRA = for | | | | | | | |
| 1 | 95010_at | Cluster Incl U21050 Tnf receptor-associated factor 3/cds = (180,1883)/gb = U21050/gi = 719292/ug = Mm.27431/len = 2359/ | P | 255.2 | P | 226.4 | P | 223.1 | P |
| | | STRA = for | | | | | | | |
| 1 | 95563_at | Cluster Incl AJ130977 Mus musculus mRNA for Ariadne protein, partial/cds = (0,1394)/gb = AJ130977/gi = 3925718/ | P | 115.3 | P | 3.8 | P | 205.1 | P |
| | | ug = Mm 27783/len = 1629/STRA = for | | | | | | | |
| 1 | 96183_at | Cluster Incl AW122985: UI-M-BH2 1-apb-b-02-0-UI s1 Mus musculus cDNA, 3 end/clone = UI-M-BH2 1-apb-b-02-0-UI/ | P | 172.3 | P | 198.4 | P | 252 | P |
| | | clone_end = 3/gb = AW122985/gi = 6098515/ug = Mm 28459/len = 427/STRA = for | | | | | | | |
| 1 | 96219_at | Cluster Incl AI850357: UI-M-BG1-aig-b-01-0-UI s1 Mus musculus cDNA. 3 end/clone = UI-M-BG1-aig-b-01-0-UI/ | P | 801.2 | P | 713.4 | P | 927 | P |
| | | clone_end = 3/gb = AIB50357/gi = 5494263/ug = Mm 28702/len = 456/STRA = for | | | | | | | |
| 1 | 96725_at | Cluster Incl AI838398. UI-M-AJ0-aav-h-02-0-UI s2 Mus musculus cDNA, 3 end/clone = UI-M-AJ0-aav-h-02-0-UI/ | P | 513.6 | P | 426.5 | P | 592.7 | P |
| | | clone_end = 3/gb = AI838398/gi = 5472611/ug = Mm 28833/len = 260/STRA = for | | | | | | | |
| 1 | 96749_f_at | Cluster Incl AA619554 vo88e08 r1 Mus musculus cDNA, 5 end/clone = IMAGE-1066214/clone_end = 5/gb = AA619554/ | P | 24 | P | 41.8 | P | 53.4 | P |
| | | gi = 2523430/ug = Mm 28958/len = 468/STRA = for | | | | | | | |
| 1 | 96832_at | Cluster Incl AA606878 vm87a04 r1 Mus musculus cDNA, 5 end/clone = IMAGE-1005198/clone_end = 5/gb = AA606878/ | P | 717.5 | P | 816.8 | P | 963.8 | P |
| | | gi = 2455771/ug = Mm 29419/len = 223/STRA = for | | | | | | | |
| 1 | 97392_at | Cluster Incl AW050086 UI-M-BH1-amm-g-09-0-UI s1 Mus musculus cDNA, 3 end/clone = UI-M-BH1-amm-g-09-0-UI/ | P | 449 | P | 508.1 | P | 543 | P |
| | | clone_end = 3/gb = AW050086/gi = 5910615/ug = Mm 29809/len = 442/STRA = for | | | | | | | |
| 1 | 97395_at | Cluster Incl AW122465 UI-M-BH2 2-aou-c-10-0-UI s1 Mus musculus cDNA, 3 end/clone = UI-M-BH2 2-aou-c-10-0-UI/ | P | 816.5 | P | 886.2 | P | 996.3 | P |
| | | clone_end = 3/gb = AW122465/gi = 6097985/ug = Mm 29835/len = 221/STRA = for | | | | | | | |
| 1 | 97917_at | Cluster Incl Y13778 Mus musculus GCN5L1 gene, exon 1 and joined CDS/cds = (70,447)/gb = Y13778/gi = 2769584/ | P | 1120.4 | P | 1121.3 | P | 1299.1 | P |
| | | ug = Mm 30118/len = 448/STRA = for | | | | | | | |
| 1 | 97957_at | Cluster Incl AF072759 Mus musculus fatty acid transport protein 4 mRNA, partial cds/cds = (0,1520)/gb = AF072769/ | P | 497.3 | P | 451.3 | P | 649.7 | P |
| | | gi = 3335568/ug = Mm.30487/len = 1521/STRA = for | | | | | | | |
| 1 | 98439_at | Cluster Incl AW049358 UI-M-BH1-ane-a-03-0-UI s1 Mus musculus cDNA, 3 end/clone = UI-M-BH1-ane-a-03-0-UI/ | P | 363.2 | P | 369.4 | P | 494.9 | P |
| | | clone_end = 3/gb = AW049356/gi = 5909885/ug = Mm 34490/len = 437/STRA = for | | | | | | | |
| 1 | 99033_at | Cluster Incl AI837130 UI-M-AK0-adc-g-01-0-UI.s1 Mus musculus cDNA, 3 end/clone = UI-M-AK0-adc-g-01-0-UI/ | P | 944.3 | P | 1052 | P | 1223.3 | P |
| | | clone_end = 3/gb = AI837130/gi = 5471343/ug = Mm.39038/len = 406/STRA = for | | | | | | | |
| 1 | 99068_at | Cluster Incl X80169 M musculus mRNA for 200 kD protein/cds = (252,6086)/gb = X80169/gi = 562764/ug = Mm 3989/ | P | 379.7 | P | 291.6 | P | 474.9 | P |
| | | len = 6331/STRA = for | | | | | | | |
| 1 | 99445_at | Cluster Incl AW047012 UI-M-BH1-alp-g-12-0-UI s1 Mus musculus cDNA, 3 end/clone = UI-M-BH1-alp-g-12-0-UI/ | P | 303.2 | P | 341 | P | 425.1 | P |
| | | clone_end = 3/gb = AW047012/gi = 5907541/ug = Mm 4042/len = 373/STRA = for | | | | | | | |
| 1 | 99492_at | Cluster Incl AI851990. UI-M-BH0-aiq-e-01-0-UI.s1 Mus musculus cDNA, 3 end/clone = UI-M-BH0-aiq-e-01-0-UI/ | P | 365.9 | P | 424.4 | P | 494.3 | P |
| | | clone_end = 3/gb = AI851990/gi = 5495896/ug = Mm.41550/len = 404/STRA = for | | | | | | | |
| 1 | 100010_at | Cluster Incl U36340 Mus musculus CACCC-box binding protein BKLF mRNA, complete cds/cds = (44,1078)/gb = U36340/ | P | 135.9 | P | 346.3 | P | 530.7 | P |
| | | gi = 1244514/ug = Mm 4549/len = 1200/STRA = for | | | | | | | |
| 1 | 101837_g_at | Cluster Incl D45859 Protein phosphatase 1B, magnesium dependent, beta isoform/cds = (0,461)/gb = D45859/gi = 961469/ | P | 217.3 | P | 226.6 | P | 310.3 | P |
| | | ug = Mm 849/len = 546/STRA = for | | | | | | | |
| 1 | 101858_at | Cluster Incl AF015770 Mus musculus radical Innge (radical-fringe) mRNA, complete cds/cds = (0,998)/gb = AF015770/ | P | 498 | P | 618.8 | P | 570.4 | P |
| | | gi = 2454573/ug = Mm 871/len = 999/STRA = for | | | | | | | |
| 1 | 101933_at | Cluster Incl AF035646 Mus musculus small GTPase Rab10 (Rab10) mRNA, complete cds/cds = (241,843)/gb = AF035646/ | P | 552.6 | P | 327.1 | P | 648.5 | P |
| | | gi = 3406427/ug = Mm 9455/len = 897/STRA = for | | | | | | | |
| 1 | 101938_at | Cluster Incl X75959 M. musculus mRNA for polyA binding protein (testis-enriched isoform)/cds = (163,2049)/gb = X75959/ | P | 261.3 | P | 357.4 | P | 395.2 | P |
| | | gi = 473091/ug = Mm 9493/len = 2135/STRA = for | | | | | | | |
| 1 | 102306_at | Cluster Incl AF060178 Mus musculus heparan sulfate 2-sulfotransferase (Hs2st) mRNA, complete cds/cds = (37,1107)/ | P | 211.3 | P | 214.9 | P | 304.8 | P |
| | | gb = AF060178/gi = 3228535/ug = Mm 12863/len = 2057/STRA = for | | | | | | | |
| 1 | 102960_at | Cluster Incl X96618 M. musculus mRNA for novel stromal cell protein/cds = (96,761)/gb = X96618/gi = 1370221/ | P | 203 | P | 271.5 | P | 388.7 | P |
| | | ug = Mm.17958/len = 1031/STRA = for | | | | | | | |
| 1 | 103219_at | Cluster Incl AI327518: mb43a09 y1 Mus musculus cDNA, 5 end/clone = IMAGE-332152/clone_end = 5/gb = AI327518/ | P | 561.5 | P | 459.7 | P | 813.7 | P |
| | | gi = 4061947/ug = Mm 19941/len = 423/STRA = for | | | | | | | |
| 1 | 103439_at | Cluster Incl AW045417: UI-M-BH1-akr-a-01-0-UI.s1 Mus musculus cDNA, 3 end/clone = UI-M-BH1-akr-a-01-0-UI/ | P | 347.7 | P | 783.3 | P | 1087 | P |
| | | clone_end = 3/gb = AW045417/gi = 5905946/ug = Mm 214/len = 336/STRA = rev | | | | | | | |
| 1 | 103451_at | Cluster Incl AI835159: UI-M-AQ0-aaf-e-10-0-UI S1 Mus musculus cDNA, 3 end/clone = UI-M-AQ0-aaf-e-10-0-UI/ | P | 268.6 | P | 295.1 | P | 368.2 | P |
| | | clone_end = 3/gb = AI835159/gi = 5469372/ug = Mm 21613/len = 139/STRA = for | | | | | | | |
| 1 | 103751_at | Cluster Incl AA833096 uc95g05.r1 Mus musculus cDNA, 5 end/clone = IMAGE-1433432/clone_end = 5/gb = AA833096/ | P | 1036.3 | P | 1239.9 | P | 1633 | P |
| | | gi = 2906824/ug = Mm 24508/len = 289/STRA = for | | | | | | | |
| 1 | 103790_at | Cluster Incl AI846632: UI-M-AQ1-aef-h-03-0-UI.s1 Mus musculus cDNA, 3 end/clone = UI-M-AQ1-aef-h-03-0-UI/ | P | 887.9 | P | 887.6 | P | 1064.5 | P |
| | | clone_end = 3/gb = AI846632/gi = 5490538/ug = Mm.24950/len = 322/STRA = for | | | | | | | |
| 1 | 103971_at | Cluster Incl X84896: M. musculus mRNA for ATP receptor/cds = (164,1363)/gb = X84896/gi = 1166454/ug = Mm.25722/ | P | 354.3 | P | 402.5 | P | 557 | P |
| | | len = 1760/STRA = for | | | | | | | |
| 1 | 104467_at | Cluster Incl AA763004: vw60b02 r1 Mus musculus cDNA, 5 end/clone = IMAGE-1248171/clone_end = 5/gb = AA763004/ | P | 619.8 | P | 343 | P | 588.6 | P |
| | | gl = 2812751/ug = Mm 2975/len = 545/STRA = for | | | | | | | |
| 1 | 104492_at | Cluster Incl U92702 Early B-cell factor 3/cds = (73,1863)/gb = U92702/gi = 2114427/ug = Mm 30282/len = 3015/STRA = rev | P | 253.5 | P | 311 | P | 367.3 | P |
| 1 | 92193_r_at | Cluster Incl D12619 Proprotein convertase subtilisin/kexin type 5/cds = (69,2816)/gb = D12619/gi = 220565/ug = Mm.3401/ | P | 262.9 | P | 209.3 | P | 473.1 | P |
| | | len = 2994/STRA = for | | | | | | | |
| 1 | 92211_at | Cluster Incl AF013969 Mus musculus antigen containing epitope to monoclonal antibody MMS-85/12 mRNA, partial cds/ | P | 511.6 | P | 538.9 | P | 744.3 | P |
| | | cds = (0,4704)/gb = AF013969/gi = 2384710/ug = Mm.34320/len = 4957/STRA = for | | | | | | | |
| 1 | 92225_f_at | Cluster Incl U58280: Mus musculus second largest subunit of RNA polymerase I (RPA2) mRNA, complete cds/ | P | 455.2 | P | 382.5 | P | 793.5 | P |
| | | cds = (55,3462)/gb = U58280/gi = 1621593/ug = Mm 34589/len = 3978/STRA = for | | | | | | | |
| 1 | 92294_at | Cluster Incl AW060793 UI-M-BH1-anu-g-02-0-UI s1 Mus musculus cDNA, 3 end/clone = UI-M-BH1-anu-g-02-0-UI/ | P | 287.7 | P | 202.4 | P | 326.6 | P |
| | | clone_end = 3/gb = AW060793/gi = 6008544/ug = Mm 3727/len = 453/STRA = rev | | | | | | | |
| 1 | 92908_at | Cluster Incl L36829 Mus musculus alphaA-crystallin-binding protein 1 (alphaA-CRYBP1) gene/cds = (103,8169)/gb = L36829/ | P | 78.3 | P | 104.5 | P | 160.3 | P |
| | | gi = 818007/ug = Mm 4808/len = 8601/STRA = rev | | | | | | | |
| 1 | 93144_at | Cluster Incl AI854602: UI-M-BH0-ake-c-05-0-UI s1 Mus musculus cDNA, 3 end/clone = UI-M-BH0-ake-c-05-0-UI/ | P | 851.9 | P | 553.8 | P | 658 | P |
| | | clone_end = 3/gb = AI854602/gi = 5498508/ug = Mm 5197/len = 481/STRA = for | | | | | | | |
| 1 | 93693_at | Cluster Incl AI835373: UI-M-AQ0-aah-h-10-0-UI s1 Mus musculus cDNA, 3 end/clone = UI-M-AQ0-aah-h-10-0-UI/ | P | 271.8 | P | 387.3 | P | 508.2 | P |
| | | clone_end = 3/gb = AI835373/gi = 5469586/ug = Mm 844/len = 285/STRA = for | | | | | | | |
| 1 | 94099_at | Cluster Incl AF049124 Mus musculus neuronal pentraxin 2 (Nptx2) mRNA, complete cds/cds = (67,1356)/gb = AF049124/ | P | 159.2 | P | 72.9 | P | 263.4 | P |
| | | gi = 2935463/ug = Mm 10099/len = 1608/STRA = for | | | | | | | |
| 1 | 94125_at | Cluster Incl AF030454 Epithelial V-like antigen/cds = (174,821)/gb = AF030454/gi = 3169827/ug = Mm 10715/len = 1279/ | P | 265.2 | P | 266.8 | P | 407.7 | P |
| | | STRA = for | | | | | | | |
| 1 | 94132_at | Cluster Incl X03920 M musculus GSHPx gene/cds = (37,642)/gb = X03920/gi = 51123/ug = Mm 1090/len = 923/STRA = for | P | 1220.5 | P | 1191.8 | P | 1604.9 | P |
| 1 | 94706_s_at | Cluster Incl AI327488 mb01a01 yt Mus musculus cDNA, 5 end/clone = IMAGE-318888/clone_end = 5/gb = AI327488/ | P | 539.9 | P | 648.6 | P | 726.5 | P |
| | | gi = 4061917/ug = Mm 13854/len = 420/STRA = for | | | | | | | |
| 1 | 95326_at | Cluster Incl AF042180.Testis-specific protein, Y-encoded-like/cds = (55,1194)/gb = AF042180/gi = 3688779/ug = Mm 20445/ | P | 343.4 | P | 396.6 | P | 541.6 | P |
| | | len = 1455/STRA = for | | | | | | | |
| 1 | 95351_at | Clusler Incl AA414339 vc66b04.s1 Mus musculus cDNA, 5 end/clone = IMAGE-779503/clone_end = 5/gb = AA414339/ | P | 48 | P | 102.5 | P | 78 | P |
| | | gi = 2074486/ug = Mm.21121/len = 411/STRA = for | | | | | | | |
| 1 | 95400_l_at | Cluster Incl AI447783 mq84c02x1 Mus musculus cDNA, 3 end/clone = IMAGE-585410/clone_end = 3/gb = AI447783/ | P | 370.2 | P | 328.9 | P | 442.4 | P |
| | | gi = 4306405/ug = Mm 23936/len = 347/3TRA = rev | | | | | | | |
| 1 | 95992_at | Cluster Incl C79950 C79950 Mus musculus cDNA, 3 end/clone = J0074C04/clone_end = 3/gb = C79950/gi = 2520280/ | P | 846.2 | P | 710.4 | P | 789.1 | P |
| | | ug = Mm 25056/len = 530/STRA = rev | | | | | | | |
| 1 | 96546_r_at | Cluster Incl AI447510 mp69a04x1 Mus musculus cDNA, 3 end/clone = IMAGE-574446/clone_end = 3/gb = AI447510/ | P | 1244.3 | P | 1482.7 | P | 2474.3 | P |
| | | gi = 4304492/ug = Mm 28/len = 291/STRA = for | | | | | | | |
| 1 | 96569_at | Cluster Incl AA268298 mz63a12 r1 Mus musculus cDNA, 5 end/clone = IMAGE-718078/clone_end = 5/gb = AA266298/ | P | 578.8 | P | 731.8 | P | 854.6 | P |
| | | gi = 1902420/ug = Mm 2972/len = 172/STRA = for | | | | | | | |
| 1 | 97083_at | Cluster Incl AA600468.vm78h03 r1 Mus musculus cDNA, 5 end/clone = IMAGE-1004405/clone_end = 5/gb = AA600468/ | P | 368 | P | 361.8 | P | 600.7 | P |
| | | gi = 2434093/ug = Mm 31138/len = 283/STRA = for | | | | | | | |
| 1 | 97163_g_at | Cluster Incl AI563699 vj83d06 y1 Mus musculus cDNA, 5 end/clone = lMAGE-935627/clone_end = 5/gb = AI563699/ | P | 565.3 | P | 641.5 | P | 679.4 | P |
| | | gi = 4522156/ug = Mm 35500/len = 392/STRA = rev | | | | | | | |
| 1 | 97176_at | Cluster Incl AI645547: vt97b07.x1 Mus musculus cDNA, 3 end/clone = IMAGE-1179061/clone_end = 3/gb = AI645547/ | P | 618.3 | P | 733.4 | P | 943.5 | P |
| | | gi = 4724022/ug = Mm 35777/len = 493/STRA = rev | | | | | | | |
| 1 | 97179_at | Clusler Incl C76543 C76543 Mus musculus cDNA, 3 end/clone = J0013B08/clone_end = 3/gb = C76543/gi = 2516873/ | P | 289.6 | P | 259 | P | 351.8 | P |
| | | ug = Mm 35801/len = 448/STRA = rev | | | | | | | |
| 1 | 97723_at | Cluster Incl X62537 Chromobox homolog 2 (Drosophila Pc class)/cds = (147,1706)/gb = X62537/gi = 52976/ug = Mm 3908/ | P | 330.1 | P | 323 | P | 429.5 | P |
| | | len = 1872/STRA = for | | | | | | | |
| 1 | 98344_f_at | Cluster Incl AI850438 UI-M-BG1-aih-a-04-0-UI s1 Mus musculus cDNA, 3 end/clone = UI-M-BG1-aih-a-04-0-UI/ | P | 1266.8 | P | 1364.2 | P | 1435.3 | P |
| | | clone_end = 3/gb = AI850438/gi = 5494344/ug = Mm 43257/len = 304/STRA = rev | | | | | | | |
| 1 | 98778_at | Cluster Incl AI837543.UI-M-AL0-abs-d-04-0-UI s1 Mus musculus cDNA, 3 end/clone = UI-M-AL0-abs-d-04-0-UI/ | P | 238.1 | P | 223.7 | P | 316.2 | P |
| | | clone_end = 3/gb = AI837543/gi = 5471756/ug = Mm 4681/len = 246/STRA = for | | | | | | | |
| 1 | 99389_at | Cluster Incl M21673: Mouse productively rearranged T-cell receptor-beta (LH8-1) gene (V5 2-D2.1-J2 5 region), complete | P | 171.3 | P | 166.4 | P | 221 | P |
| | | cds/cds = (0,347)/gb = M21673/gi = 201830/ug = Mm.56958/len = 348/STRA = for | | | | | | | |
| 1 | 99847_at | Cluster Incl X73523 M musculus mRNA for Gal beta1, 3GalNAc alpha2,3-sialyltransferase/cds = (6,1019)/gb = X73523/ | P | 268.2 | P | 205 | P | 313.2 | P |
| | | gi = 402214/ug = Mm.6284/len = 1405/STRA = for | | | | | | | |
| 1 | 100344_at | Cluster Incl AI464141 vc34b10 x1 Mus musculus cDNA, 3 end/clone = IMAGE-776443/clone_end = 3/gb = AI464141/ | P | 900.4 | P | 836 | P | 1376.7 | P |
| | | gi = 4318171/ug = Mm 88145/len = 475/STRA = rev | | | | | | | |
| 1 | 100387_g_at | Cluster Incl AB019600 Mus musculus mRNA for caspase9, complete cds/cds = (0,1364)/gb = AB019600/gi = 6440941/ | P | 425 | P | 445.3 | P | 447.5 | P |
| | | ug = Mm 88829/len = 1365/STRA = for | | | | | | | |
| 1 | 101344_at | Cluster Incl AF019371 Cholecystokinin B receptor/cds = (0,1361)/gb = AF019371/gi = 2460176/ug = Mm.57011/len = 1362/ | P | 707 | P | 554.8 | P | 777.2 | P |
| | | STRA = for | | | | | | | |
| 1 | 96464_at | Cluster Incl N28179 MDB1515 Mus musculus cDNA, 3 end/clone_end = 3/gb = N28179/gi = 1146035/ug = Mm.556/len = 284/ | P | 634.7 | P | 821.3 | P | 1328 | P |
| | | STRA = rev | | | | | | | |
| 1 | 97004_at | Cluster Incl AJ133429.Mus musculus or17 gene/cds = (0,938)/gb = AJ133429/gi = 5869924/ug = Mm 88846/len = 939/ | P | 1020.2 | P | 1265 | P | 1537.6 | P |
| | | STRA = for | | | | | | | |
| 1 | 97655_at | Cluster Incl Y11169 Desmocollin 3/cds = UNKNOWN/gb = Y11169/gi = 2765094/ug = Mm 89935/len = 2903/STRA = for | P | 121.8 | P | 82.8 | P | 172.5 | P |
| 1 | 97666_r_at | Cluster Incl C76213: C76213 Mus musculus cDNA, 3 end/clone = J0006A04/clone_end = 3/gb = C76213/gi = 2516543/ | P | 122.4 | P | 98.8 | P | 184.6 | P |
| | | ug = Mm.89955/len = 605/STRA = rev | | | | | | | |
| 1 | 100717_at | Cluster Incl U90926: Mus musculus putative TNF-resistance related protein mRNA, complete cds/cds = (136,492)/ | P | 399.3 | P | 524.4 | P | 712.6 | P |
| | | gb = U90926/gi = 1906634/ug = Mm 2548/len = 540/STRA = for | | | | | | | |
| 1 | 101226_at | Cluster Incl C77776 C77776 Mus musculus cDNA, 3 end/clone = J0037D08/clone_end = 3/gb = C77776/gi = 2518106/ | P | 657.3 | P | 784.1 | P | 827.4 | P |
| | | ug = Mm 5650/len = 622/STRA = rev | | | | | | | |
| 1 | 102478_f_at | Cluster Incl AW122990 UI-M-BH2 1-apb-b-09-0-UI.s1 Mus musculus cDNA, 3 end/clone = UI-M-BH2 1-apb-b-09-0-UI/ | P | 298.1 | P | 280.3 | P | 369.8 | P |
| | | clone_end = 3/gb = AW122990/gi = 6098520/ug = Mm 48699/len = 387/STRA = rev | | | | | | | |
| 1 | 94279_at | Cluster Incl AW125279 UI-M-BH2 1-apy-a-05-0-UI s1 Mus musculus cDNA, 3 end/clone = UI-M-BH2 1-apy-a-05-0-UI/ | P | 191.8 | P | 152.6 | P | 216.3 | P |
| | | clone_end = 3/gb = AW125279/gi = 6100809/ug = Mm 22514/len = 303/STRA = for | | | | | | | |
| 1 | 94886_at | Cluster Incl L18888 Calnexin/cds = (0,1775)/gb = L18888/gi = 309131/ug = Mm 23929/len = 1776/STRA = rev | P | 389.4 | P | 391.4 | P | 493.7 | A |
| 1 | 95486_at | Cluster Incl AA794134.vr42h04 s1 Mus musculus cDNA, 5 end/clone = IMAGE-1123351/clone_end = 5/gb = AA794134/ | P | 447.6 | P | 411.8 | P | 555.8 | A |
| | | gi = 2857089/ug = Mm 24997/len = 155/STRA = for | | | | | | | |
| 1 | 96023_at | Cluster Incl AI852472 UI-M-BHO-aip-f-10-0-UI s1 Mus musculus cDNA, 3 end/clone = UI-M-BH0-aip-l-10-0-UI/ | P | 121 | A | 100.8 | P | 158.6 | P |
| | | clone_end = 3/gb = AI852472/gi = 5496376/ug = Mm 25727/len = 402/STRA = rev | | | | | | | |
| 1 | 96032_at | Cluster Incl L19737.ATP synthase, H + transporting, mitochondrial F0 complex, subunit c (subunit 9), isoform 1/ | P | 96.3 | P | 105.5 | A | 203.5 | P |
| | | cds = (3,413)/gb = L19737/gi = 309287/ug = Mm 258/len = 499/STRA = for | | | | | | | |
| 1 | 96719_l_at | Cluster Incl X59382 Parvalbumin/cds = (56,388)/gb = X59382/gi = 509138/ug = Mm 2766/len = 615/STRA = for | P | 1130.9 | P | 1179.8 | P | 1147.9 | A |
| 1 | 97234_at | Cluster Incl AA792453: vs69d09 r1 Mus musculus cDNA, 5 end/clone = IMAGE-1151537/clone_end = 5/gb = AA792453/ | P | 128.3 | P | 203.7 | M | 221.9 | P |
| | | gi = 2855408/ug = Mm 27822/len = 351/STRA = for | | | | | | | |
| 1 | 97508_at | Cluster Incl M29462.Malate dehydrogenase, soluble/cds = (81,1085)/gb = M29462/gi = 199094/ug = Mm 3156/len = 1288/ | P | 404.6 | P | 410.6 | A | 578.2 | P |
| | | STRA = for | | | | | | | |
| 1 | 101981_at | Cluster Incl U09419 Mus musculus retinoid X receptor interacting protein (RIP15) mRNA, complete cds/cds = (130,1470)/ | P | 164.2 | P | 193.3 | M | 318.6 | P |
| | | gb = U09419/gi = 691713/ug = Mm. 968/len = 1841/STRA = for | | | | | | | |
| 1 | 102804_at | Cluster Incl X67279 Biliary glycoprotein 1/cds = UNKNOWN/gb = X67279/gi = 50170/ug = Mm 14114/len = 2922/STRA = for | P | 687 | P | 575 | A | 997.7 | P |
| 1 | 103013_at | Cluster Incl X77602 Upstream transcription factor 2/cds = (0,1028)/gb = X77602/gi = 609171/ug = Mm 15781/len = 1029/ | A | 503.5 | P | 637.4 | P | 640.1 | P |
| | | STRA = rev | | | | | | | |
| 1 | 103843_at | Cluster Incl M36777 Guanine nucleotide binding protein, alpha o/cds = (21,1085)/gb = M36777/gi = 193359/ug = Mm.20964/ | M | 270.6 | P | 343.2 | P | 440.5 | P |
| | | len = 1910/STRA = for | | | | | | | |
| 1 | 104629_at | Cluster Incl AI876546 uj60a09 x1 Mus musculus cDNA, 3 end/clone = IMAGE-1924312/clone_end = 3/gb = AI876546/ | P | 418.5 | P | 502.2 | P | 576 | P |
| | | gi = 5550595/ug = Mm 24331/len = 338/STRA = for | | | | | | | |
| 1 | 95575_r_at | Cluster Incl AI641995 ub74e05x1 Mus musculus cDNA, 3 end/clone = IMAGE-1383468/clone_end = 3/gb = AI641995/ | P | 205.3 | A | 195.4 | P | 300.6 | P |
| | | gi = 4720470/ug = Mm 27821/len = 385/STRA = for | | | | | | | |
| 1 | 95593_at | Cluster Incl AW125446 UI-M-BH2.3-aqh-h-05-0-UI s1 Mus musculus cDNA, 3 end/done = UI-M-BH2 3-aqh-h-05-0-UI/ | P | 689.3 | P | 914.1 | P | 836.4 | A |
| | | clone_end = 3/gb = AW125446/gi = 6100976/ug = Mm 27902/len = 540/STRA = for | | | | | | | |
| 1 | 95610_at | Cluster Incl AA636547 vq76h11 s1 Mus musculus cDNA, 5 end/clone = IMAGE-1108293/clone_end = 5/gb = AA636547/ | P | 125 | P | 81 | A | 166.7 | P |
| | | gi = 2560326/ug = Mm 27989/len = 460/STRA = for | | | | | | | |
| 1 | 96126_at | Cluster Incl AF036894 Sphingosine phosphate lyase 1/cds = (60,1766)/gb = AF036894/gi = 2906010/ug = Mm.28119/ | P | 368.7 | P | 350.3 | P | 433 | P |
| | | len = 1911/STRA = for | | | | | | | |
| 1 | 96790_f_at | Cluster Incl AW125222: UI-M-BH2.1-aps-c-12-0-UI s1 Mus musculus cDNA, 3 end/clone = UI-M-BH2.1-aps-c-12-0-UI/ | P | 338.3 | P | 353.3 | P | 381.4 | P |
| | | clone_end = 3/gb = AW125222/gi = 6100752/ug = Mm 29098/len = 396/STRA = for | | | | | | | |
| 1 | 97437_f_at | Cluster Incl AW122483: UI-M-BH2.2-aou-f-01-0-UI.s1 Mus musculus cDNA, 3 end/clone = UI-M-BH2.2-aou-f-01-0-UI/ | A | 76.5 | P | 74.6 | P | 75.7 | P |
| | | clone_end = 3/gb = AW122483/gi = 6098005/ug = Mm30115/len = 277/STRA = for | | | | | | | |
| 1 | 99528_at | Cluster Incl AW122015.UI-M-BH2 3-aoj-h-12-0-UI s1 Mus musculus cDNA, 3 end/clone = UI-M-8H2 3-aoj-h-12-0-UI/ | P | 149 | P | 121.2 | P | 143.6 | P |
| | | clone_end = 3/gb = AW122015/gi = 6097421/ug = Mm 42151/len = 405/STRA = for | | | | | | | |
| 1 | 100941_at | Cluster Incl AF039567 Mus musculus Msx-interacting-zinc finger protein 1 (Miz1) mRNA, complete cds/cds = (26,1498)/ | P | 138.6 | P | 141.5 | A | 218.5 | P |
| | | gb = AF039567/gi = 2773147/ug = Mm 6370/len = 1847/STRA = for | | | | | | | |
| 1 | 102251_at | Cluster Incl AF047714 Mus musculus melastatin mRNA, complete cds/cds = (307,1935)/gb = AF047714/gi = 3047241/ | P | 359.3 | P | 493.2 | P | 644.9 | M |
| | | ug = Mm.10752/len = 2722/STRA = for | | | | | | | |
| 1 | 102681_at | Cluster Incl M24377 Early growth response 2/cds = (0,1262)/gb = M24377/gi = 198599/ug = Mm 1353/len = 1263/STRA = for | P | 689.3 | P | 613.1 | P | 968.2 | P |
| 1 | 103706_at | Cluster Incl AI036406 ub65e10 r1 Mus musculus cDNA, 5 end/clone = IMAGE-1382634/clone_end = 5/gb = AI036406/ | P | 217.7 | P | 223.8 | P | 308.8 | M |
| | | gi = 3260097/ug = Mm 24078/len = 380/STRA = for | | | | | | | |
| 1 | 104245_at | Cluster Incl AW124639 UI-M-BH2 1-apw-c-08-0-UI s1 Mus musculus cDNA, 3 end/clone = UI-M-BH2.1-apwc-08-0-UI/ | P | 872.9 | P | 839.4 | P | 1264 | M |
| | | clone_end = 3/gb = AW124839/gi = 6100169/ug = Mm28288/len = 413/STRA = rev | | | | | | | |
| 1 | 104463_at | Cluster Incl AI157789 ue58d04 r1 Mus musculus cDNA, 5 end/clone = IMAGE-1495303/clone_end = 5/gb = AI157789/ | P | 236.7 | P | 259.6 | P | 357.1 | P |
| | | gi = 3686258/ug = Mm 29631/len = 443/STRA = for | | | | | | | |
| 1 | 104503_at | Cluster Incl AI563623 vx92d09.x1 Mus musculus cDNA, 3 end/clone = IMAGE-1282673/clone_end = 3/gb = AI563623/ | P | 92.7 | P | 32.7 | P | 151.9 | A |
| | | gi = 4522080/ug = Mm 30648/len = 482/STRA = for | | | | | | | |
| 1 | 92461_at | Cluster IncL AB021224-Mus musculus mRNA for membrane-type-4 matrix metalloproteinase, complete cds/cds = (85,1848)/ | P | 328.1 | P | 411.3 | P | 725.5 | P |
| | | gb = AB021224/gi = 5688930/ug = Mm 42047/len = 3490/STRA = for | | | | | | | |
| 1 | 93872_at | Cluster Incl AF014117 Mus musculus GDNF receptor alpha (GDNFR-alpha) mRNA, complete cds/cds = (913,2319)/ | P | 43.3 | P | 53.1 | A | 80.4 | P |
| | | gb = AF014117/gi = 2624960/ug = Mm.88367/len = 2549/STRA = for | | | | | | | |
| 1 | 94095_at | Cluster lncl AI552528 vx23a11.x1 Mus musculus cDNA, 3 end/clone = IMAGE-1265276/clone_end = 3/gb = AI552528/ | P | 219.9 | M | 272.8 | P | 316.2 | P |
| | | gi = 4484891/ug = Mm 100313/len = 496/STRA = rev | | | | | | | |
| 1 | 95891_at | Cluster lncl AI591977.mt35c08 y1 Mus musculus cDNA, 5 end/clone = IMAGE-623054/clone_end = 5/gb = AI591977/ | P | 266.6 | P | 267 | P | 407.4 | P |
| | | gi = 4601025/ug = Mm 24058/len = 354/STRA = rev | | | | | | | |
| 1 | 97726_at | Cluster Incl L35029 Glutamate receptor, ionotropic, NMDA2C (epsilon 3)/cds = (112,3723)/gb = L35029/gi = 602752/ | P | 739.8 | P | 532.9 | P | 703.9 | M |
| | | ug = Mm 39090/len = 3999/STRA = for | | | | | | | |
| 1 | 98771_at | Cluster Incl L25890 Eph receptor B2/cds = (92,2983)/gb = L25890/gi = 414593/ug = Mm.4652/len = 3104/STRA = for | P | 267.3 | P | 272.9 | P | 281.5 | A |
| 1 | 98853_at | Cluster Incl D30779: Mouse mRNA for phospholipase A2 receptor, complete cds/cds = (131,4594)/gb = D30779/gi = 1375042/ | P | 172.1 | P | 141.5 | A | 233.9 | P |
| | | ug = Mm.5092/len = 5569/STRA = for | | | | | | | |
| 1 | 99415_at | Cluster Incl U43321: Frizzled homolog 8, (Drosophila)/cds = (0,2057)/gb = U43321/gi = 1151259/ug = Mm.57055/len = 2058/ | P | 128.2 | P | 73.3 | A | 81.8 | P |
| | | STRA = for | | | | | | | |
| 1 | 100295_at | Cluster Incl M34597 Mouse lg germline lambda-chain gene Vx-J2-C2-region, complete cds, clone 30X2/cds = (0,368)/ | P | 410.6 | P | 497.9 | P | 633.9 | P |
| | | gb = M34597/gi = 197929/ug = Mm.86392/len = 369/STRA = for | | | | | | | |
| 1 | 94623_at | Cluster Incl U82698: Mlls musculus enameiln mRNA, complete cds/cds = (300,4124)/gb = U82698/gi = 2737960/ | P | 182.5 | P | 167.3 | P | 284.9 | A |
| | | ug = Mm 8014/len = 4170/STRA = for | | | | | | | |
| 1 | 95848_at | Cluster Incl C76063: C76063 Mus musculus cDNA, 3 end/clone = J0003A08/clone_end = 3/gb = C76063/gi = 2516393/ | P | 178.2 | P | 264.5 | P | 295.8 | P |
| | | ug = Mm 24888/len = 626/STRA = rev | | | | | | | |
| 1 | 96996_at | Cluster Incl AJ245706 Mus musculus mRNA for nicotinic acetylcholine receptor subunit alpha6 (NICA6 gene)/ | P | 192.6 | P | 174.6 | P | 322.7 | P |
| | | cds = (80,1564)/gb = AJ245706/gi = 5777608/ug = Mm 88637/len = 1595/STRA = for | | | | | | | |
| 1 | 101595_at | Cluster Incl C78787 C78787 Mus musculus cDNA, 3 end/clone = J0055A07/clone_end = 3/gb = C78787/gi = 2979447/ | P | 108.6 | A | 179.1 | P | 254.8 | P |
| | | ug = Mm 5751/len = 387/STRA = rev | | | | | | | |
| 1 | 96310_at | Cluster Incl L07508 Mouse Golli-mpb mRNA, complete cds (alternate transcript from clone BG21)/cds = (274,861)/ | P | 139.8 | A | 156.1 | A | 283.4 | P |
| | | gb = L07508/gi = 193586/ug = Mm 2992/len = 1407/STRA = for | | | | | | | |
| 1 | 103062_at | Cluster Incl AB004664.Mus musculus DNA for Rab33B/cds = (316,1005)/gb = AB001664/gi = 2516240/ug = Mm 1664/ | P | 184.8 | P | 183 | A | 289.6 | P |
| | | len = 3421/STRA = for | | | | | | | |
| 1 | 99497_at | Cluster Incl AI852332 UI-M-BHO-ajo-a-06-O-UI a1 Mus musculus cDNA, 3 end/clone = UI-M-BHO-ajo-a-06-0-UI/ | P | 578.1 | P | 563.9 | A | 646.5 | P |
| | | clone_end = 3/gb = AI852332/gi = 5496238/ug = Mm 41637/len = 501/STRA = for | | | | | | | |
| 1 | 99954_at | Cluster Incl AW046474 UI-M-BH1-akz-a-08-0-UI s1 Mus musculus cDNA, 3 end/clone = UI-M-BH1-akz-a-08-0-UI/ | P | 516.1 | P | 655.9 | P | 826.7 | P |
| | | clone_end = 3/gb = AW046474/gi = 5907003/ug = Mm 439/len = 499/STRA = for | | | | | | | |
| 1 | 100405_at | Cluster Incl X56683 Mouse mRNA coding for modifier 2 protein/cds = (0,521)/gb = X56683/gi = 53164/ug = Mm 46536/ | P | 198.1 | P | 135.6 | P | 208.3 | A |
| | | len = 522/STRA = rev | | | | | | | |
| 1 | 102231_at | Cluster Incl AB017614 Mus musculus mRNA for OASIS protein, complete cds/cds = (27,1550)/gb = AB017614/gi = 4519620/ | P | 880.1 | P | 966.1 | A | 1043.4 | P |
| | | ug = Mm.10353/len = 1908/STRA = rev | | | | | | | |
| 1 | 102711_at | Cluster Incl U85055: Mus musculus rap1/rap2 interacting protein mRNA, complete cds/cds = (0,1643)/gb = U85055/ | P | 369.9 | P | 348.4 | P | 454 | A |
| | | gi = 1814395/ug = Mm 1426/len = 2208/STRA = rev | | | | | | | |
| 1 | 103275_at | Cluster Incl U13836 Mus musculus vacuolar adenosine triphosphatase subunit Ac116 mRNA, complete cds/cds = (18,2537)/ | P | 165.1 | P | 142.7 | A | 220 | A |
| | | gb = U13836/gi = 3955095/ug = Mm.20869/len = 2557/STRA for | | | | | | | |
| 1 | 103766_at | Cluster Incl X97817 Semaphorin F/cds = (27,3260)/gb = X97817/gi = 1419018/ug = Mm 24733/len = 3949/STRA = for | P | 31.7 | P | 17.3 | M | 60.9 | P |
| 1 | 93206_g_at | Cluster Incl M14167 Mouse ADA gene encoding adenosine deaminase, promoter region/cds = (142,144)/gb = M14167/ | P | 193 | P | 239.7 | P | 264.1 | M |
| | | gi = 191688/ug = Mm.56978/len = 145/STRA = for | | | | | | | |
| 1 | 93211_at | Cluster Incl AF032115 Mus musculus cysteine string protein mRNA complete cds/cds = (0,596)/gb = AF032115/ | P | 376.1 | P | 379.3 | P | 634.4 | P |
| | | gi = 2642628/ug = Mm 57022/len = 597/STRA = for | | | | | | | |
| 1 | 95295_s_at | Cluster Incl X59398.FMS-like tyrosine kinase 3/cds = (81,3083)/gb = X59398/gi = 50978/ug = Mm 194/len = 3521/STRA = for | P | 322.2 | P | 281 | P | 289.3 | M |
| 1 | 96557_at | Cluster Incl AB010833 Patched homolog 2/cds = (0,3548)/gb = AB010833/gi = 2842557/ug = Mm 28529/len = 3549/STRA = for | P | 381.8 | P | 455.5 | A | 504.8 | P |
| 1 | 97097_at | Cluster Incl AW125669: UI-M-BH2 2-aqi-c-12-0-UI S1 Mus musculus cDNA, 3 end/clone = UI-M-BH2 2-aqi-c-12-0-UI/ | P | 225.1 | P | 253.9 | A | 278.6 | P |
| | | clone_end = 3/gb = AW125669/gi = 6101199/ug = Mm 3183/len = 507/STRA = for | | | | | | | |
| 1 | 97136_at | Cluster IncI AA571388 vl77a12 s1 Mus musculus cDNA, 3 end/clone = IMAGE-978238/clone_end = 3/gb = AA571388/ | P | −23 | A | −13.4 | P | 68.5 | P |
| | | gi = 2346317/ug = Mm 34930/len = 440/STRA = for | | | | | | | |
| 1 | 98349_at | Cluster Incl X62646 M musculus mRNA for IL-6 signal transducer (gp130)/cds = (88,2841)/gb = X62646/gi = 840816/ | P | 12.2 | A | −3.5 | M | 128.7 | P |
| | | ug = Mm 4364/len = 2995/STRA = for | | | | | | | |
| 1 | 100750_at | Cluster IncI AA414903 vc50b09.r1 Mus musculus cDNA, 3 end/clone = IMAGE-777977/clone_end = 3/gb = AA414903/ | A | 208 | A | 164.1 | P | 249.4 | P |
| | | gi = 2075133/ug = Mm 3808/len = 151/STRA = rev | | | | | | | |
| 1 | 101661_r_at | Cluster Incl AA420337 vc51dO3 r1 Mus musculus cDNA, 3 end/clone = IMAGE-778085/clone_end = 3/gb = AA420337/ | P | 35.6 | M | 39.7 | P | 31.1 | P |
| | | gi = 2080894/ug = Mm.6761/len = 281/STRA = rev | | | | | | | |
| 1 | 94072_g_at | Cluster Incl AI847904 UI-M-AI1-afl-a-09-0-UI.s1 Mus musculus cDNA, 3 end/clone = UI-M-AI1-afl-o-09-0-UI/clone_end = 3/ | P | 578.1 | P | 580.8 | P | 874.2 | A |
| | | gb = AI847904/gi = 5491810/ug = Mm 22114/len = 350/STRA = for | | | | | | | |
| 1 | 97222_at | Cluster Incl AI845921 UI-M-AK1-aex-d-11-0-UI s1 Mus musculus cDNA, 3 end/clone = UI-M-AK1-aea-d-11-0-UI/ | P | 517.5 | P | 399.3 | A | 749.5 | P |
| | | clone_end = 3/gb = AI845921/gi = 5489827/ug = Mm.27756/len = 298/STRA = for | | | | | | | |
| 1 | 97821_at | Cluster Incl AI646056 vw54c11.x1 Mus musculus cDNA, 3 end/clone = IMAGE-1247636/clone_end = 3/gb = AI646056/ | P | 500.8 | A | 472.9 | P | 475.3 | A |
| | | gi = 4724531/ug = Mm 28202/len = 561/STRA = rev | | | | | | | |
| 1 | 100619_r_at | Cluster Incl AA062013 mj83h01 r1 Mus musculus cDNA, 5 end/clone = IMAGE-482737/clone_end = 5/gb = AA062013/ | P | 241.1 | P | 403.6 | P | 427.2 | P |
| | | gi = 1555829/ug = Mm 658/len = 529/STRA = for | | | | | | | |
| 1 | 95545_at | Cluster Incl X04480.lnsulin-like growth factor 1/cds = (152,535)/gb = X04480/gi = 51801/ug = Mm 2770/len = 696/STRA = for | P | 342.5 | P | 554.7 | P | 749.4 | A |
| 1 | 98443_at | Cluster Incl AI882416 uc01c09 r1 Mus musculus cDNA, 5 end/clone = IMAGE-1396720/clone_end = 5/gb = AI882416/ | P | 495.9 | P | 487.2 | A | 721.8 | P |
| | | gi = 5567505/ug = Mm 34519/len = 338/STRA = for | | | | | | | |
| 1 | 99463_at | Cluster Incl X63023 Cytochrome P450, steroid Inducible 3a13/cds = (83,1594)/gb = X63023/gi = 50634/ug = Mm 4094/ | P | 92 | P | 85.1 | M | 170.9 | M |
| | | len = 1881/STRA = for | | | | | | | |
| 1 | 100020_at | Cluster Incl J04036 Solute carrier family 4 (anlon exchanger), member 2/cds = (182,3895)/gb = J04036/gi = 192132/ | P | 375.5 | P | 404.8 | A | 430.4 | A |
| | | ug = Mm 4580/len = 4088/STRA = for | | | | | | | |
| 1 | 101417_at | Cluster Incl AF020191: Mus musculus proline-rich protein 13 mRNA, partial cds/cds = (0,511)/gb = AF020191/gi = 2501952/ | P | 246.5 | P | 202.2 | A | 339.5 | M |
| | | ug = Mm.7412/len = 1370/STRA = for | | | | | | | |
| 1 | 101840_at | Cluster Incl L06864 Mus musculus epidermal growth factor receptor (EGFR) mRNA, partial cds/cds = (0,363)/gb = L06864/ | P | 80 | A | 79 | A | 161.7 | P |
| | | gi = 193001/ug = Mm 8534/len = 364/STRA = for | | | | | | | |
| 1 | 102242_at | Cluster Incl AF050182 Mus musculus PERIOD 3 (Per3) mRNA, complete cds/cds = (357,3698)/gb = AF050182/gi = 3136149/ | M | 510.5 | P | 555.4 | P | 731.7 | P |
| | | ug = Mm 10723/len = 4863/STRA = for | | | | | | | |
| 1 | 104238_at | Cluster Incl U60881: ADP-ribosyltransferase 5/cds = (382,1311)/gb = U60881/gi = 1522688/ug = Mm.2816/len = 1547/ | P | 247.4 | P | 236.4 | A | 354.7 | P |
| | | STRA = for | | | | | | | |
| 1 | 92745_at | Cluster Incl AB005458 Homeo box A9/cds = (446,763)/gb = AB005458/gi = 3080549/ug = Mm 4694/len = 764/STRA = for | P | 70.1 | P | 96 | M | 104.7 | M |
| 1 | 93689_at | Cluster Incl AW122071 UI-M-BH2 2-aoo-e-12-0-UI s1 Mus musculus cDNA, 3 end/clone = UI-M-BH2 2-aoo-e-12-0-UI/ | P | 514.2 | P | 416.6 | M | 535.1 | A |
| | | clone_end = 3/gb = AW122071/gi = 6097478/ug = Mm 840/len = 413/STRA = for | | | | | | | |
| 1 | 94135_at | Cluster Incl M76987 Transcription factor CP2/cds = UNKNOWN/gb = M76987/gi = 191802/ug = Mm 1139/len = 4023/ | P | 343.4 | P | 378.2 | M | 433.7 | P |
| | | STRA = for | | | | | | | |
| 1 | 94171_at | Cluster Incl AF051367 Mus musculus pactolus mRNA, complete cds/cds = (63,2279)/gb = AF051367/gi = 3287490/ | M | 230.7 | P | 246.5 | M | 233.9 | A |
| | | ug = Mm 12872/len = 2628/STRA = for | | | | | | | |
| 1 | 97096_at | Cluster Incl J02935 Mouse cAMP-dependent protein kinase type II regulatory subunit mRNA, 3 end/cds = (0,1205)/ | P | 448.2 | P | 407.3 | P | 506.6 | P |
| | | gb = J02935/gi = 200356/ug = Mm 3176/len = 1533/STRA = for | | | | | | | |
| 1 | 98867_at | Cluster Incl AI848793 UI-M-AJ1-ahg-d-12-0-UI s1 Mus musculus cDNA, 3 end/clone = UI-M-AJ1-ahg-d-12-0-UI/ | P | 99.8 | M | 155.6 | P | 160.9 | A |
| | | clone_end = 3/gb = AI848793/gi = 5492699/ug = Mm 5152/len = 388/STRA = rev | | | | | | | |
| 1 | 99810_at | Cluster Incl X91864 Glutathione peroxidase 2, pseudogene 1/cds = (0,158)/gb = X91864/gi = 1359980/ug = Mm 57225/ | P | 305.2 | P | 178 | P | 391.1 | M |
| | | len = 159/STRA = for | | | | | | | |
| 1 | 102557_at | Cluster Incl U62673 Mus musculus histone H2a(A)-613, histone H2a(B)-613, and histone H2b-613 (H2b) genes, complete | P | 86.7 | P | 105.1 | P | 123.1 | P |
| | | cds/cds = (0,392)/gb = U62673/gi = 1458138/ug = Mm 57213/len = 393/STRA = for | | | | | | | |
| 1 | 94551_at | Cluster Incl AI853061 UI-M-BH0-ajn-g-12-0-UI s1 Mus musculus cDNA, 3 end/clone = UI-M-BH0-ajn-g-12-0-UI/ | P | 194.2 | P | 237 | A | 363.1 | P |
| | | clone_end = 3/gb = AI853061/gi = 5496967/ug = Mm 29087/len = 363/STRA = for | | | | | | | |
| 1 | 94556_at | Cluster Incl AI746846 uI08b08.y1 Mus musculus cDNA, 5 end/clone = IMAGE-2076183/clone_end = 5/gb = AI746846/ | P | 144.9 | P | 82.5 | A | 130.2 | A |
| | | gi = 5125110/ug = Mm 29101/len = 596/STRA = for | | | | | | | |
| 1 | 102372_at | Cluster Incl M90766 Mouse Ig active joining chain (j chain) mRNA of the b allele, complete cds/cds = (21,500)/gb = M90766/ | A | 186.5 | A | 197.9 | P | 179.9 | P |
| | | gi = 194473/ug = Mm.1192/len = 501/STRA = for | | | | | | | |
| 1 | 103614_at | Cluster Incl AW047899 UI-M-BH1-ain-a-06-0-UI s1 Mus musculus cDNA, 3 end/clone = UI-M-BH1-aln-a-06-0-UI/ | P | 94.2 | P | −16.3 | A | 159 | A |
| | | clone_end = 3/gb = AW047B99/gi = 5908428/ug = Mm 20225/len = 403/STRA = rev | | | | | | | |
| 1 | 103840_at | Cluster Incl AJ01,1923 Mus musculus mRNA for cell cycle checkpoint protein/cds = (75,2138)/gb = AJ011923/gi = 3758877/ | A | 112.9 | P | 132.9 | A | 282.2 | A |
| | | ug = Mm 20947/len = 2640/STRA = for | | | | | | | |
| 1 | 104036_at | Cluster Incl AI845447 UI-M-A01-aej-f-01-0-UI s1 Mus musculus cDNA, 3 end/clone = UI-M-AO1-aej-f-01-0-UI/ | M | 186.4 | P | 109.4 | A | 183.7 | M |
| | | clone_end = 3/gb = AI845447/gi = 5489353/ug = Mm 21440/len = 343/STRA = for | | | | | | | |
| 1 | 96806_at | Cluster Incl AI843802 UI-M-AN1-afc-e-12-0-UI.s1 Mus musculus cDNA, 3 end/clone = UI-M-AN1-afc-e-12-0-UI/ | P | 362.4 | P | 297.7 | A | 512.1 | A |
| | | clone_end = 3/gb = AI843802/gi = 5478015/ug = Mm 29209/len = 537/STRA = for | | | | | | | |
| 1 | 98457_at | Cluster Incl AF020195 Mus musculus pancreas sodium bicarbonate cotransporter mRNA, complete cds/cds = (29,3268)/ | P | 68.6 | P | 105.4 | A | 168.8 | P |
| | | gb = AF020195/gi = 3298571/ug = Mm.34747/len = 3337/STRA = for | | | | | | | |
| 1 | 102265_at | Cluster Incl X59060 Myogenic factor 6/cds = (25,753)/gb = X59060/gi = 53297/ug = Mm 11/len = 1258/STRA = for | P | 198.1 | P | 227.8 | A | 306.7 | A |
| 1 | 102891_at | Cluster Incl D86527: Werner syndrome homolog (human)/cds = (183,4388)/gb = D86527/gi = 2130974/ug = Mm 15446/ | P | 245.2 | P | 195.6 | A | 259.6 | A |
| | | len = 4955/STRA = rev | | | | | | | |
| 1 | 104273_at | Cluster Incl U95215 Bile acid CoA-amino acid n-acyltransferase/cds = (189,1451)/gb = U95215/gi = 2130557/ug = Mm.2859/ | P | 219.4 | M | 225.2 | M | 325.5 | P |
| | | len = 1870/STRA = rev | | | | | | | |
| 1 | 92895_at | Cluster Incl AF038857 untitled/cds = (0,2297)/gb = AF038857/gi = 2952265/ug = Mm.4774/len = 2298/STRA = for | P | 116.1 | P | 87.1 | P | 170.7 | A |
| 1 | 93185_at | Cluster Incl AW045808 UI-M-BH1-akt-h-04-0-UI.s1 Mus musculus cDNA, 3 end/clone = UI-M-BH1-akt-h-04-0-UI/ | M | 161.2 | A | 163 | P | 205.9 | M |
| | | clone_end = 3/gb = AW0458O8/gi = 5906337/ug = Mm 5473/len = 367/STRA = rev | | | | | | | |
| 1 | 94762_at | Cluster Incl U92972 Mus musculus protease-activated receptor 3 (PAR3) mRNA, complete cds/cds = (152,1261)/ | P | 230.1 | M | 318.3 | M | 352.7 | P |
| | | gb = U92972/gi = 1938376/ug = Mm 1612/len = 2409/STRA = rev | | | | | | | |
| 1 | 102580_r_at | Cluster Incl M11988 Homeo box A6/cds = (0,293)/gb = M11988/gi = Mm.57244/len = 294/STRA = for | P | 140.9 | A | 166.2 | P | 250 | M |
| 1 | 101233_at | Cluster Incl C78616.C78616 Mus musculus cDNA, 3 end/clone = J0052A12/clone_end = 3/gb = C78616/gi = 2518946/ | M | 53.8 | P | 24.1 | A | 64.3 | M |
| | | ug = Mm 5725/len = 541/STRA = rev | | | | | | | |
| 1 | 101687_r_at | Cluster Incl C79518.C79518 Mus musculus cDNA,3 end/clone = J0067E04/clone_end = 3/gb = C79518/gi = 2519848/ | P | 44.7 | A | 79.3 | A | 114.9 | P |
| | | ug = Mm 36431/len = 252/STRA = rev | | | | | | | |
| 1 | AFFX- | E. Coli BioD 3 prime | A | 134.1 | P | 178.9 | A | 218.2 | A |
| | BioDn-3_at | | | | | | | | |
| 1 | 93788_at | Cluster Incl M37831.Cytochrome C oxidase, subunit IV/cds = (1,510)/gb = M37831/gi = 459880/ug = Mm 2136/len = 610/ | P | 126.1 | P | 133 | A | 142.5 | A |
| | | STRA = for | | | | | | | |
| 1 | 94235_at | Cluster Incl AW050034 UI-M-BH1-amm-b-10-0-UI s1 Mus musculus cDNA, 3 end/clone = UI-M-BH1-amm-b-10-0-UI/ | A | 54.3 | P | 8.5 | A | 28 | P |
| | | clone_end = 3/gb = AW050034/gi = 5910563/ug = Mm.22327/len = 531/STRA = for | | | | | | | |
| 1 | 96044_at | Cluster Incl AF067660: Bcl2-like 10/cds = (142,717)/gb = AF067660/gi = 3955265/ug = Mm 25988/len = 1225/STRA = for | M | 188.2 | P | 195.1 | M | 497 | P |
| 1 | 98526_r_at | Cluster Incl AI854606 UI-M-BH0-ake-c-09-0-UI s1 Mus musculus cDNA, 3 end/clone = UI-M-BH0-ake-c-09-0-UI/ | M | 252.1 | M | 240.3 | M | 378.2 | P |
| | | clone_end = 3/gb = AI854606/gi = 5498512/ug = Mm 35829/len = 472/STRA = rev | | | | | | | |
| 1 | 102765_at | Cluster Incl AF071317-Mus musculus COP9 complex subunit 7b (COPS7b) mRNA, complete cds/cds = (130,924)/ | P | 317.8 | P | 369.2 | P | 514.5 | M |
| | | gb = AF071317/gi = 3309175/ug = Mm 12973/len = 1990/STRA = for | | | | | | | |
| 1 | 102771_at | Cluster Incl AF091628 Mus musculus ERG-associated protein ESET mRNA, complete cds/cds = (101,4024)/gb = AF091628/ | A | 393.4 | P | 197.8 | A | 377.8 | P |
| | | gi = 3644041/ug = Mm 13162/len = 4602/STRA = rev | | | | | | | |
| 1 | 102861_at | Cluster Incl AF028739 Imprinted polyspecific membrane transporter-like gene 1/cds = (179,1399)/gb = AF02B739/ | A | 100.2 | P | 159 | A | 270.2 | M |
| | | gi-2583224/ug = Mm 15093/len = 1466/STRA = for | | | | | | | |
| 1 | 96184_at | Cluster Incl AIB35242.UI-M-AQ0-aaa-h-10-0-UI s1 Mus musculus cDNA, 3 end/clone = UI-M-AQ0-aaa-h-10-0-UI/ | A | 668.1 | A | 667.8 | A | 905.8 | A |
| | | clone_end = 3/gb = AI835242/gi = 5489455/ug = Mm 28462/len = 294/STRA = for | | | | | | | |
| 1 | 98970_at | Cluster Incl AB016044 Mus musculus mRNA for GGPP synthase, complete cds/cds = (107,1009)/gb = AB016044/ | P | 70.1 | A | 111.6 | P | 133.5 | A |
| | | gi = 4586887/ug = Mm 36520/len = 1025/STRA = for | | | | | | | |
| 1 | 100961_at | Cluster Incl AF012871.Long(electocardiographic) QT syndrome 2/cds = (321,3809)/gb = AF012871/gi = 2582014/ | P | 338.5 | P | 337.5 | P | 421.5 | M |
| | | ug = Mm 6539/len = 4221/STRA = for | | | | | | | |
| 1 | 101399_at | Cluster Incl M77174 Perlecan (heparan sulfate proteoglycan 2)/cds = (578,11701)/gb = M77174/gi = 200295/ug = Mm 7257/ | P | 301.4 | P | 492.2 | M | 535.6 | P |
| | | len = 12667/STRA = for | | | | | | | |
| 1 | 101942_at | Cluster Incl AA839050: vw47a01.r1 Mus musculus cDNA, 5 end/clone = IMAGE-1246920/clone_end = 5/gb = AA839050/ | P | 292.9 | P | 386 | P | 456.8 | A |
| | | gi = 2915145/ug = Mm.854/len = 511/STRA = for | | | | | | | |
| 1 | 104239_at | Cluster Incl AW208513: uo60102.x1 Mus musculus cDNA, 3 end/clone = IMAGE-2646939/clone_end = 3/gb = AW208513/ | P | 37.7 | A | 21.6 | A | 109.5 | A |
| | | gi = 6514453/ug = Mm 28172/len = 461/STRA = for | | | | | | | |
| 1 | 104500_at | Cluster Incl L34111: Alpha-L-iduronidase/cds = (26,1930)/gb = L34111/gi = 623169/ug = Mm 3054/len = 3109/STRA = for | A | 66.6 | P | 107.4 | A | 173.4 | P |
| 1 | 92271_at | Cluster Incl X63963 Paired box gene 6/cds = (162,1472)/gb = X63963/gi = 460050/ug = Mm 3608/len = 2481/STRA = for | P | 472 | P | 475.5 | A | 713.9 | M |
| 1 | 92458_at | Cluster Incl AJ003133 Mus musculus mRNA for ORC1-related protein, complete cds/cds = (123,2645)/gb = AJ003133/ | P | 177.7 | M | 103.7 | A | 222.7 | P |
| | | gi = 4034784/ug = Mm 42024/len = 3017/STRA = for | | | | | | | |
| 1 | 94177_at | Cluster Incl Y15733 Mus musculus mRNA for 17-beta-hydroxysteroid dehydrogenase type 7/cds = (03,1067)/gb = Y15733 | A | 3.8 | A | −7.1 | A | 177 | P |
| | | gi = 3319970/ug = Mm 12882/len = 1756/STRA = for | | | | | | | |
| 1 | 96593_at | Cluster Incl X87257: ELK1, member of ETS oncogene family/cds = (473,1762)/gb = X87257/gi = 836634/ug = Mm 3064/ | A | 108.5 | P | 100.5 | A | 119.6 | A |
| | | len = 2286/STRA = for | | | | | | | |
| 1 | 102078_at | Cluster Incl AF066075 untitled/cds = (127,474)/gb = AF066075/gi = 4092929/ug = Mm 42082/len = 714/STRA = for | P | 32.2 | P | 34.4 | A | 48.4 | A |
| 1 | 94541_at | Cluster Incl AI839549 UI-M-AN0-acn-h-08-0-UI.s1 Mus musculus cDNA, 3 end/clone = UI-M-AN0-acn-h-08-0-UI/ | P | 177.2 | P | 147.1 | A | 233.9 | M |
| | | clone_end = 3/gb = AI839549/gi = 5473762/ug = Mm 29065/len = 162/STRA = for | | | | | | | |
| 1 | 101513_at | Cluster Incl AI840454 UI-M-AN0-aci-c-01-o-UI s1 Mus musculus cDNA, 3 end/clone = UI-M-AN0-aci-c-01-o-UI/ | A | 127.2 | P | 151.4 | M | 173.3 | P |
| | | clone_end = 3/gb = AI840454/gi = 5474667/ug = Mm.836/len = 446/STRA = for | | | | | | | |
| 1 | 102995_s_at | Cluster Incl M13226 Granzyme A/cds = (198,971)/gb = M13226/gi = 201048/ug = Mm.15510/len = 1039/STRA = for | P | 219.9 | A | 141.3 | P | 243.4 | A |
| 1 | 103361_at | Cluster Incl AI853856 UI-M-BH0-ajr-e-10-0-UI.s1 Mus musculus cDNA, 3 end/clone = UI-M-BH0-ajr-e-10-0-UI/ | P | 39.6 | P | 12.2 | A | 58.9 | A |
| | | clone_end = 3/gb = AI853856/gi = 5497762/ug = Mm 18506/len = 374/STRA = rev | | | | | | | |
| 1 | 104581_at | Cluster Incl AI845438 UI-M-AO1-aej-d-10-0-UI s1 Mus musculus cDNA, 3 end/clone = UI-M-AO1-aoj-d-10-0-UI/ | P | 36.3 | A | 21.5 | A | 59.1 | P |
| | | clone_end = 3/gb = AI845438/gi = 5489339/ug = Mm 23975/len = 369/STRA = for | | | | | | | |
| 1 | 104708_at | Cluster Incl AI463490 vw62d08.x1 Mus musculus cDNA, 3 end/clone = IMAGE-1248399/clone_end = 3/gb = AI463490/ | A | 220.2 | P | 263.7 | A | 348.5 | A |
| | | gi = 4317520/ug = Mm 24408/len = 180/STRA = for | | | | | | | |
| 1 | 102649_s_at | Cluster Incl D64162 Mouse clone Rae-123 mRNA for cell surface protein, complete cds/cds = (92,853)/gb = D84162/ | P | 155.2 | P | 129.3 | A | 191.8 | A |
| | | gi = 1311547/ug = Mm 1326/len = 1242/STRA = for | | | | | | | |
| 1 | 104449_at | Cluster Incl X81202: Glycine receptor, beta subunit/cds = (0,1455)/gb = X81202/gi = 557654/ug = Mm 29422/len = 1465/ | P | 51 | A | 153 | P | 139 | A |
| | | STRA = for | | | | | | | |
| 1 | 104509_at | Cluster Incl AF059213 Mus musculus cholesterol 25-hydroxylase mRNA, complete cds/cds = (9,905)/gb = AF059213/ | P | 136.6 | A | 108.1 | A | 247.2 | P |
| | | gi = 4038305/ug = Mm 30824/len = 1365/STRA = rev | | | | | | | |
| 1 | 92312_at | Cluster Incl U55772 Mus musculus p170 phosphatidylinositol 3-kinase mRNA, complete cds/cds = (57,4586)/gb = U55772/ | P | 122 | A | 100.8 | A | 149.9 | A |
| | | gi = 1305537/ug = Mm 3810/len = 5067/STRA = for | | | | | | | |
| 1 | 92493_at | Cluster Incl Y10926 M musculus mRNA for transcription factor RBP-L/cds = (103,1650)/gb = Y10926/gi = 2052118/ | A | 156.7 | P | 161.9 | A | 286.2 | A |
| | | ug = Mm 42177/len = 2456/STRA = for | | | | | | | |
| 1 | 93452_at | Cluster Incl AF031896 Cerberus 1/cds = (82,900)/gb = AF031896/gi = 2654026/ug = Mm 6780/len = 1752/STRA = for | A | 163.3 | P | 105.5 | M | 243.1 | P |
| 1 | 97143_at | Cluster Incl C79450 C79450 Mus musculus cDNA, 3 end/clone = J0066E08/clone_end = 3/gb = C79450/gi = 2519780/ | A | 95.1 | A | 139 | P | 279.6 | A |
| | | ug = Mm 34943/len = 579/STRA = rev | | | | | | | |
| 1 | 97156_at | Cluster Incl D32200 Mouse gene for Hes-3/cds = (0,527)/gb = D32200/gi = 699611/ug = Mm 3516/len = 528/STRA = for | P | 102 | A | 255.2 | A | 207.6 | A |
| 1 | 97701_at | Cluster Incl AI181906 ud73e11 x1 Mus musculus cDNA, 3 end/clone = IMAGE-1451564/clone_end = 3/gb = AI181906/ | A | 136 | P | 106.8 | A | 135.2 | P |
| | | gi = 3732544/ug = Mm 38004/len = 387/STRA = rev | | | | | | | |
| 1 | 99893_at | Cluster Incl AF020737: Fibroblast growth factor 13/cds = (9,743)/gb = AF020737/gi = 2444476/ug = Mm 7995/len = 817/ | P | 44.3 | A | 81.9 | A | 98.3 | M |
| | | STRA = for | | | | | | | |
| 1 | 102621_at | Cluster Incl AF090866 Mus musculus CDO (Cdo) mRNA, complete cds/cds = (29,3781)/gb = AF090866/gi = 3644025/ | A | 293.9 | A | 207.1 | A | 270.5 | M |
| | | ug = Mm 20439/len = 3914/STRA = for | | | | | | | |
| 1 | 97665_1_at | Cluster Incl C76213 C76213 Mus musculus cDNA, 3 end/clone = J0006A04/clone_end = 3/gb = C76213/gi = 2516543/ | P | 124.8 | A | −40.4 | A | 158.9 | A |
| | | ug = Mm 89955/len = 605/STRA = rev | | | | | | | |
| 1 | 101614_at | Cluster Incl AA672848 vp57b02 r1 Mus musculus cDNA, 3 end/clone = IMAGE-1080747/clone_end = 3/gb = AA672648/ | P | 118.8 | P | 68.5 | A | 126.1 | A |
| | | gi = 2644865/ug = Mm 6126/len = 404/STRA = rev | | | | | | | |
| 1 | 102143_at | Cluster Incl AW047018: UI-M-BH1-alp-h-06-0-UI s1 Mus musculus cDNA, 3 end/clone = UI-M-BH1-alp-h-06-0-UI/ | P | 213.7 | M | 239.9 | M | 325.2 | M |
| | | clone_end = 3/gb = AW047018/gi = 5907547/ug = Mm 44276/len = 396/STRA = rev | | | | | | | |
| 2 | 92573_at | Cluster Incl AB021743 Mus musculus mRNA for PR65 (type 2A serine/threonine protein phosphatase A subunit), complete | P | 1742.8 | P | 2202.3 | P | 2358.5 | P |
| | | cds/cds = (0,1769)/gb = AB021743/gi = 4514638/ug = Mm. 1034/len = 1770/STRA = for | | | | | | | |
| 2 | 92599_at | Cluster Incl AF029843 Mus musculus phosphoglycerate mutase muscle-specific subunit mRNA, complete cds/ | P | 463.9 | P | 867.5 | P | 1623.7 | P |
| | | cds = (45,806)/gb = AF029843/gi = 3043914/ug = Mm 10738/len = 836/STRA = for | | | | | | | |
| 2 | 92621_at | Cluster Incl X97982 M musculus mRNA for poly(C)-binding protein, splice variant E/cds = (221,1270)/gb = X97982/ | P | 2366.9 | P | 2737.1 | P | 2736.6 | P |
| | | gi = 1360002/ug = Mm.111/len = 1655/STRA = rev | | | | | | | |
| 2 | 92637_at | Cluster Incl J03928 Phosphofructokinase, liver, B-type/cds = (42,2384)/gb = J03928/gi = 200297/ug = Mm 1166/len = 2688/ | P | 781.8 | P | 677.1 | P | 683 | P |
| | | STRA = for | | | | | | | |
| 2 | 92775_at | Cluster Incl AA656757: vr50c03.s1 Mus musculus cDNA, 5 end/clone = IMAGE-1124068/clone_end = 5/gb = AA656757/ | P | 583.4 | P | 804.7 | P | 886.7 | P |
| | | gi = 2592911/ug = Mm 12267/len = 407/STRA = for | | | | | | | |
| 2 | 92777_at | Cluster Incl M32490 Insulin-like growth factor binding protein 10/cds = (189,1328)/gb = M32490/gi = 192909/ug = Mm 1231/ | P | 106.8 | P | 104.3 | P | 109.2 | P |
| | | len = 2018/STRA = for | | | | | | | |
| 2 | 92787_at | Cluster Incl AI845902: UI-M-AK1-aex-b-07-0-UI s1 Mus musculus cDNA, 3 end/clone = UI-M-AK1-aex-b-07-0-UI/ | P | 457 | P | 652.2 | P | 913.2 | P |
| | | clone_end = 3/gb = AI845902/gi = 5489808/ug = Mm.12459/len = 358/STRA = for | | | | | | | |
| 2 | 93104_at | Cluster Incl Z16410 M musculus btg1 mRNA/cds = (0,515)/gb = Z16410/gi = 50187/ug = Mm 16596/len = 516/STRA = for | P | 1510.9 | P | 1331.1 | P | 1945.4 | P |
| 2 | 93285_at | Cluster Incl AI845584 UI-M-AQ1-adx-c-06-0-UI s1 Mus musculus cDNA, 3 end/clone = UI-M-AQ1-adx-c-06-0-UI/ | P | 604.9 | P | 546.2 | P | 596.8 | P |
| | | clone_end = 3/gb = AI845584/gi = 5489490/ug = Mm 1791/len = 254/STRA = for | | | | | | | |
| 2 | 93346_at | Cluster Incl M15668.Mus musculus X chromosome-linked phosphoglycerate kinase (pgk-1) mRNA, complete cds/ | P | 1835 | P | 2188.7 | P | 2313.2 | P |
| | | cds = (22,1275)/gb = M15668/gi = 202422/ug = Mm 188/len = 1653/STRA = for | | | | | | | |
| 2 | 93528_s_at | Cluster Incl AI848050 UI-M-AH1-agp-g-10-0-UI.s1 Mus musculus cDNA, 3 end/clone = UI-M-AH1-agp-g-10-0-UI/ | P | 458.1 | P | 620.7 | P | 1483.1 | P |
| | | clone_end = 3/gb = AI848050/gi = 5491956/ug = Mm 19788/len = 377/STRA = for | | | | | | | |
| 2 | 93728_at | Cluster Incl X62940: Transforming growth factor beta 1 induced transcript 4/cds = (186,617)/gb = X62040/gi = 54928/ | P | 1006.6 | P | 898.8 | P | 1065.8 | P |
| | | ug = Mm.20927/len = 1706/STRA = for | | | | | | | |
| 2 | 94048_at | Cluster Incl AW120683: UI-M-BH2.3-anz-c-06-0-UI.s1 Mus musculus cDNA, 3 end/clone = UI-M-BH2.3-anz-c-06-0-UI/ | P | 441.8 | P | 650.3 | P | 578.6 | P |
| | | clone_end = 3/gb = AW120683/gi = 6095981/ug = Mm 21981/len = 420/STRA = for | | | | | | | |
| 2 | 94052_at | Cluster Incl AB013360 Mus musculus mRNA for DPM2, complete cds/cds = (24,278)/gb = AB013360/gi = 3790360/ | P | 821.5 | P | 862 | P | 1050.5 | P |
| | | ug = Mm 22001/len = 279/STRA = for | | | | | | | |
| 2 | 94236_at | Cluster Incl AI838152 UI-M-AL0-abw-e-06-0-UI a1 Mus musculus cDNA, 3 end/clone = UI-M-AL0-abw-e-06-0-UI/ | P | 1728.1 | P | 1611.2 | P | 1918.2 | P |
| | | clone_end = 3/gb = AI838152/gi = 5472365/ug = Mm 22330/len = 451/STRA = for | | | | | | | |
| 2 | 94255_g_at | Cluster Incl AI845237 UI-M-BG0-aht-g-09-0-UI s1 Mus musculus cDNA, 3 end/clone = UI-M-BG0-aht-g-09-0-UI/ | P | 898.1 | P | 1121.5 | P | 1020.8 | P |
| | | clone_end = 3/gb = AI845237/gi = 5489143/ug = Mm 22409/len = 255/STRA = for | | | | | | | |
| 2 | 94261_at | Cluster Incl AI836625. UI-M-AP0-abh-a-12-0-UI.s1 Mus musculus cDNA, 3 end/clone = UI-M-AP0-abh-a-12-0-UI/ | P | 1530.8 | P | 1517.4 | P | 1867.6 | P |
| | | clone_end = 3/gb = AI836625/gi = 5470838/ug = Mm 22440/len = 218/STRA = for | | | | | | | |
| 2 | 94300_f_at | Cluster Incl AI839497: UI-M-AN0-acn-b-04-0-UI s1 Mus musculus cDNA, 3 end/clone = UI-M-AN0-acn-b-04-0-UI/ | P | 826.1 | P | 893.5 | P | 1060 | P |
| | | clone_end = 3/gb = AI839497/gi = 5473710/ug = Mm 22597/len = 385/STRA = for | | | | | | | |
| 2 | 94309_g_at | Cluster Incl X70853: BM-90/fibulin extracellular matrix glyroprotein/cds = (0,353)/gb = X70853/gi = 396816/ug = Mm.22626/ | P | 1270.9 | P | 1465.9 | P | 1383.6 | P |
| | | len = 448/STRA = for | | | | | | | |
| 2 | 95468_at | Cluster Incl AI850202 UI-M-BG1-aie-b-01-0-UI s1 Mus musculus cDNA, 3 end/clone = UI-M-BG1-aie-b-01-0-UI/ | P | 247 | P | 346.7 | P | 327.8 | P |
| | | clone_end = 3/gb = AI850202/gi = 5494108/ug = Mm 24786/len = 401/STRA = for | | | | | | | |
| 2 | 95471_at | Cluster Incl U22399 Cyclin-dependent kinase Inhibitor 1C (P57)/cds = (137,1144)/gb = U22399/gi = 790249/ug = Mm.2480/ | P | 2899.7 | P | 4275.4 | P | 4872.5 | P |
| | | len = 1462/STRA = for | | | | | | | |
| 2 | 95474_at | Cluster Incl AW123850 UI-M-BH2 1-app-b-03-0-UI s1 Mus musculus cDNA, 3 end/clone = UI-M-BH2 1-app-b-03-0-UI/ | P | 666 | P | 354.6 | P | 618.9 | P |
| | | clone_end = 3/gb = AW123850/gi = 6099380/ug = Mm 24816/len = 347/STRA = for | | | | | | | |
| 2 | 95482_at | Cluster Incl AW124247: UI-M-BH2 1-aph-f-08-0-UI s1 Mus musculus cDNA, 3 end/clone = UI-M-BH2 1-aph-f08-0-UI | P | 827.1 | P | 816.4 | P | 828 | P |
| | | clone_end = 3/gb = AW124247/gi = 6099777/ug = Mm 24870/len = 398/STRA = for | | | | | | | |
| 2 | 96073_at | Cluster Incl U10435 Requiem/cds = (46,1161)/gb = U10435/gi = 606660/ug = Mm.2651/len = 2319/STRA = for | P | 382.4 | P | 485.8 | P | 494.8 | P |
| 2 | 96076_at | Cluster Incl AW121716: UI-M-BH2 3-aoc-h-05-0-UI s1 Mus musculus cDNA, 3 end/clone = UI-M-BH2.3-aoc-h-05-0-UI/ | P | 416.3 | P | 425.6 | P | 606.5 | P |
| | | clone_end = 3/gb = AW12176/gi = 6097049/ug = Mm 26558/len = 214/STRA = for | | | | | | | |
| 2 | 96636_at | Cluster Incl AI852649.UI-M-BHO-aiu-h-10-0-UI s1 Mus musculus cDNA, 3 end/clone = UI-M-BH0-aiu-h-10-0-UI/ | P | 318.1 | P | 385.4 | P | 567.6 | P |
| | | clone_end = 3/gb = AI852649/gi = 5496555/ug = Mm 27270/len = 388/STRA = for | | | | | | | |
| 2 | 97308_at | Cluster Incl AI835409 UI-M-AQ0-aaj-d-08-0-UI s1 Mus musculus cDNA, 3 end/clone = UI-M-AQ0-aaj-d-08-0-UI/ | P | 261.6 | P | 268 | P | 252.6 | P |
| | | clone_end = 3/gb = AI835409/gi = 5469622/ug = Mm 28063/len = 314/STRA = for | | | | | | | |
| 2 | 97813_at | Cluster Incl M61909 Avian reticuloendotheliosis viral (v-rel) oncogene homolog A/cds = (60,1709)/gb = M61909/gi = 200025/ | P | 1229.6 | P | 1372.9 | P | 1409.3 | P |
| | | ug = Mm 28170/len = 2424/STRA = for | | | | | | | |
| 2 | 97859_at | Cluster Incl AA762325 vw59d08 r1 M Mus musculus cDNA, 5 end/clone = IMAGE-1248111/clone_end = 5/gb = AA762325/ | P | 337.2 | P | 352.3 | P | 528 | P |
| | | gi = 2812072/ug = Mm 28308/len = 261/STRA = for | | | | | | | |
| 2 | 98926_at | Cluster Incl AI838601 UI-M-AO0-abz-e-11-0-UI s1 Mus musculus cDNA, 3 end/clone = UI-M-AO0-abz-e-11-0-UI/ | P | 414.4 | P | 218.3 | P | 395.6 | P |
| | | clone_end = 3/gb = AI838601/gi = 5472814/ug = Mm 28643/len = 322/STRA = for | | | | | | | |
| 2 | 98933_at | Cluster Incl AI849218 UI-M-AJ1-agz-b-02-0-UI s1 Mus musculus cDNA, 3 end/clone = UI-M-AJ1-agz-b-02-0-UI/ | P | 641.8 | P | 483.4 | P | 772.5 | P |
| | | clone_end = 3/gb = AI849218/gi = 5493124/ug = Mm 28678/len = 450/STRA = for | | | | | | | |
| 2 | 94457_at | Cluster Incl AW049735 UI-M-BH1-ani-g-08-0-UI s1 Mus musculus cDNA, 3 end/clone = UI-M-BH1-ani-g-08-0-UI/ | P | 578.2 | P | 703.5 | P | 848.7 | P |
| | | clone_end = 3/gb = AW049735/gi = 5910264/ug = Mm 28806/len = 510/STRA = for | | | | | | | |
| 2 | 94482_at | Cluster Incl AF012251.Casein kinase II, alpha 2, polypeptide/cds = (98,1150)/gb = AF012251/gi = 2738574/ug = Mm 28881/ | P | 1228.2 | P | 1454.9 | P | 1422.1 | P |
| | | len = 1630/STRA = for | | | | | | | |
| 2 | 94483_at | Cluster Incl AW060442 UI-M-BH1-anj-e-09-0-UI s1 Mus musculus cDNA, 3 end/clone = UI-M-BH1-anj-e-09-0-UI/ | P | 444.8 | P | 727.5 | P | 697.4 | P |
| | | clone_end = 3/gb = AW060442/gi = 6008193/ug = Mm 28881/len = 471/STRA = for | | | | | | | |
| 2 | 94555_at | Cluster Incl AI845579.UI-M-AQ1-adx-b-12-0-UI s1 Mus musculus cDNA, 3 end/clone = UI-M-AQ1-adx-b-12-0-UI/ | P | 832.2 | P | 770.8 | P | 1229.9 | P |
| | | clone_end = 3/gb = AI845579/gi = 5489485/ug = Mm 29092/len = 254/STRA = for | | | | | | | |
| 2 | 95044_at | Cluster Incl AI844549: UI-M-AJ1-ahf-h-10-0-UI s2 Mus musculus cDNA, 3 end/clone = UI-M-AJ1-ahf-h-10-0-UI/ | P | 516.5 | P | 650.8 | P | 498.7 | P |
| | | clone_end = 3/gb = AI844549/gi = 5488455/ug = Mm 29120/len = 430/STRA = for | | | | | | | |
| 2 | 95057_at | Cluster Incl AI846938 UI-M-AK1-aey-e-10-0-UI s1 Mus musculus cDNA, 3 end/clone = UI-M-AK1-aey-e-10-0-UI/ | P | 543.8 | P | 524.9 | P | 594 | P |
| | | clone_end = 3/gb = AI846938/gi = 5490844/ug = Mm 29151/len = 342/STRA = for | | | | | | | |
| 2 | 95081_at | Cluster Incl AI840176 UI-M-AO0-acc-f-04-0-UI s1 Mus musculus cDNA, 3 end/clone = UI-M-AO0-acc-f-04-0-UI/ | P | 625.2 | P | 632.8 | P | 2117.2 | P |
| | | clone_end = 3/gb = AI840176/gi = 5474389/ug = Mm 29253/len = 407/STRA = for | | | | | | | |
| 2 | 95128_at | Cluster Incl AI849611.UI-M-AH1-agr-g-04-0-UI s1 Mus musculus cDNA, 3 end/clone = UI-M-AH1-agr-g-04-0-UI | P | 486.5 | P | 396.7 | P | 813.3 | P |
| | | clone_end = 3/gb = AI849611/gi = 5493517/ug = Mm 29412/len = 309/STRA = for | | | | | | | |
| 2 | 95144_at | Cluster Incl AB024984.Mus musculus Sld329 mRNA, complete cds/cds = (134,1246)/gb = AB024984/gi = 5931548/ | P | 665.2 | P | 701.7 | P | 830.4 | P |
| | | ug = Mm 29453/len = 1595/STRA = for | | | | | | | |
| 2 | 95161_at | Cluster Incl AW120628 UI-M-BH2 3-anz-a-04-0-UI.s1 Mus musculus cDNA, 3 end/clone = UI-M-BH2 3-anz-a-04-0-UI/ | P | 802.7 | P | 886.3 | P | 1201.3 | P |
| | | clone_end = 3/gb = AW120628/gi = 6095961/ug = Mm.29490/len = 381/STRA = for | | | | | | | |
| 2 | 95653_at | Cluster Incl AI841323 UI-M-AM0-adv-b-03-0-UI s1 Mus musculus cDNA, 3 end/clone = UI-M-AM0-adv-b-03-0-UI/ | P | 603.2 | P | 578.9 | P | 681.9 | P |
| | | clone_end = 3/gb = AI841323/gi = 5475538/ug = Mm.29517/len = 449/STRA = for | | | | | | | |
| 2 | 95682_at | Cluster Incl AB026432 Mus musculus mRNA for XPE UV-damaged DNA binding factor, complete cds/cds = (81,3503)/ | P | 2412.8 | P | 3614.5 | P | 3351.7 | P |
| | | gb = AB026432/gi = 5931595/ug = Mm 29623/len = 4171/STRA = for | | | | | | | |
| 2 | 95692_at | Cluster Incl AI852514 UI-M-BH0-aiw-c-06-0-UI s1 Mus musculus cDNA, 3 end/clone = UI-M-BH0-aiw-c-06-0-UI/ | P | 782.9 | P | 834.7 | P | 839.2 | P |
| | | clone_end = 3/gb = AI852514/gi = 5496420/ug = Mm.29655/len = 298/STRA = for | | | | | | | |
| 2 | 95731_at | Cluster Incl AI843106 UI-M-AK1-aes-e-01-0-UI s1 Mus musculus cDNA, 3 end/clone = .UI-M-AK1-aes-e-01-0-UI/ | P | 402 | P | 217.9 | P | 427.1 | P |
| | | clone_end = 3/gb = AI843106/gi = 5477319/ug = Mm.29742/len = 302/STRA = for | | | | | | | |
| 2 | 96852_at | Cluster Incl AW122197.UI-M-BH2 2-aov-b-080-UI s1 Mus musculus cDNA, 3 end/clone = UI-M-BH2 2-aov-b-08-0-UI/ | P | 1722.1 | P | 1830.6 | P | 2126.6 | P |
| | | clone_end = 3/gb = AW122197/gi = 6097654/ug = Mm 30039/len = 436/STRA = for | | | | | | | |
| 2 | 96876_at | Cluster Incl U34259 Mus musculus Golgi 4-transmembrane spanning transporter MTP mRNA, complete cds/ | P | 3169.8 | P | 2919.5 | P | 1300.5 | P |
| | | cds = (744,1445)/gb = U34259/gi = 1463119/ug = Mm 30071/len = 1984/STRA = for | | | | | | | |
| 2 | 96926_at | Cluster Incl AA980164: ua31a05.r1 Mus musculus cDNA, 5 end/clone = IMAGE-1348304/clone_end = 5/gb = AA980164/ | P | 496.5 | P | 381.1 | P | 836.7 | P |
| | | gi = 3158700/ug = Mm.30162/len = 470/STRA = for | | | | | | | |
| 2 | 97517_at | Cluster Incl AW061222: UI-M-BH1-amj-a-02-0-UI.s2 Mus musculus cDNA, 3 end/clone = UI-M-BH1-amj-a-02-0-UI | P | 965.3 | P | 795.3 | P | 1584.2 | P |
| | | clone_end = 3/gb = AW061222/gi = 6008973/ug = Mm 3199/len = 143/STRA = for | | | | | | | |
| 2 | 98060_at | Cluster Incl D49733 Lamin A/cds = (0,1997)/gb = D49733/gi = 1838920/ug = Mm.3438 len = 2916/STRA = for | P | 1154.9 | P | 1381.7 | P | 1639.5 | P |
| 2 | 98064_at | Cluster Incl AW125378 UI-M-BH2 3-aqh-b-04-0-UI s1 Mus musculus cDNA, 3 end/clone = UI-M-BH2 3-aqh-b-04-0-UI/ | P | 737.1 | P | 759.3 | P | 823.4 | P |
| | | clone_end = 3/gb = AW125378/gi = 6100908/ug = Mm 34411/len = 447/STRA = for | | | | | | | |
| 2 | 98067_at | Cluster Incl U09507: Cyclin-dependent kinase inhibitor 1A (P21)/cds = (200,679)/gb = U09507/gi = 595302/ug = Mm.34446/ | P | 1052.1 | P | 1506.2 | P | 1552.7 | P |
| | | len = 855/STRA = for | | | | | | | |
| 2 | 98083_at | Cluster Incl AW049031 UI-M-BH1-amp-g-08-0-UI s1 Mus musculus cDNA, 3 end/clone = UI-M-BH1-amp-g-08-0-UI/ | P | 541.4 | P | 699.2 | P | 750.2 | P |
| | | clone_end = 3/gb = AW049031/gi = 5909560/ug = Mm 34576/len = 473/STRA = for | | | | | | | |
| 2 | 98140_at | Cluster Incl X60961 Cadherin 1/cds = (127,2781)/gb = X60961/gi = 55133/ug = Mm 35605/len = 4413/STRA = for | P | 713.4 | P | 933.3 | P | 1150.7 | P |
| 2 | 98579_at | Cluster Incl M28845 Early growth response 1/cds = (489,2090)/gb = M28845/gi = 1325981/ug = Mm 3765/len = 3476/ | P | 540.4 | P | 1195.7 | P | 9291 | P |
| | | STRA = rev | | | | | | | |
| 2 | 93605_at | Cluster Incl AI851163 UI-M-BH0-akg-f-05-0-UI.s1 Mus musculus cDNA, 3 end/clone = UI-M-BH0-akg-f-05-0-UI/ | P | 508.8 | P | 836.6 | P | 619.3 | P |
| | | clone_end = 3/gb = AI851163/gi = 5495034/ug = Mm.38433/len = 399/STRA = for | | | | | | | |
| 2 | 99086_g_at | Cluster Incl AI021421 ub 12a08n r1 Mus musculus cDNA, 5 end/clone = IMAGE-1366742/clone_end = 5/gb = AI021421/ | P | 89.1 | P | 82.3 | P | 232 | P |
| | | gi = 3235757/ug = Mm 38976/len = 470/STRA = for | | | | | | | |
| 2 | 99109_at | Cluster Incl M59821 Mouse growth factor-inducible protein (pip92) mRNA, complete cds/cds = (103,768)/gb = M59821/ | P | 809.5 | P | 870.1 | P | 1054.7 | P |
| | | gi = 200354/ug = Mm.399/len = 1515/STRA = for | | | | | | | |
| 2 | 99183_at | Cluster Incl AW123013 UI-M-BH2 1-apb-e-03-0-UI s1 Mus musculus cDNA, 3 end/clone = UI-M-BH2 1-apb-e-03-0-UI/ | P | 612.3 | P | 551.1 | P | 726.5 | P |
| | | clone_end = 3/gb = AW123013/gi = 6098543/ug = Mm.41840/len = 553/STRA = for | | | | | | | |
| 2 | 99566_at | Cluster Incl L31777 Triosephosphate isomerase/cds = (0,634)/gb = L31777/gi = 953213/ug = Mm 4222/len = 635/STRA = for | P | 3578.6 | P | 5051 | P | 5247.7 | P |
| 2 | 99574_at | Cluster Incl AI850887.UI-M-BH0-ajt-g-01-0-UI s1 Mus musculus cDNA, 3 end/clone = UI-M-BH0-ajt-g-01-0-UI/clone_end = 3/ | P | 303.7 | P | 405.9 | P | 430.7 | P |
| | | gb = AI850887/gi = 5494793/ug = Mm 423/len = 394/STRA = for | | | | | | | |
| 2 | 99624_at | Cluster Incl AW125517.UI-M-BH2 2-aqm-f-07-0-UI s1 Mus musculus cDNA, 3 end/clone = UI-M-BH2 2-aqm-f-07-0-UI | P | 816.9 | P | 759.4 | P | 883.1 | P |
| | | clone_end = 3/gb = AW125517/gi = 6101047/ug = Mm 43309/len = 464/STRA = for | | | | | | | |
| 2 | 99652_at | Cluster Incl AF109905 Mus musculus major histocompatibility locus class III regions Hsc701 gene, partial cds, smRNP, | P | 1050.2 | P | 833.7 | P | 1143.3 | P |
| | | G7A, NG23, MulS homolog, CLCP, NG24, NG25, and NG26 genes, complete cds, and unknown genes/cds = (0,1676)/ | | | | | | | |
| | | gb = AF109905/gi = 3986751/ug = Mm 4/STRA = for | | | | | | | |
| 2 | 99668_at | Cluster Incl U86405 Mus musculus brain amphiphysin 2 mRNA, complete cds/cds = (249,2015)/gb = U86405/gi = 1914840/ | P | 1402.7 | P | 1467.1 | P | 2347 | P |
| | | ug = Mm 4383/len = 2506/STRA = for | | | | | | | |
| 2 | 100041_at | Cluster Incl AW124133: UI-M-BH2 1-apr-d-12-0-UI s1 Mus musculus cDNA, 3 end/clone = UI-M-BH2 1-apr-d-12-0-UI/ | P | 909.6 | P | 978 | P | 1032.7 | P |
| | | clone_end = 3/gb = AW124133/gi = 6099663/ug = Mm 44226/len = 133/STRA = for | | | | | | | |
| 2 | 100056_at | Cluster Incl X54352: M domesticus MD6 mRNA/cds = (123,1391)/gb = X54352/gi = 49679/ug = Mm 4405/len = 1825/ | P | 269.8 | P | 242.3 | P | 349.9 | P |
| | | STRA = for | | | | | | | |
| 2 | 100074_at | Cluster Incl AW046723: UI-M-BH1-alt-f-03-0-UI s1 Mus musculus cDNA, 3 end/clone = UI-M-BH1-alf-f-03-0-UI/ | P | 620.6 | P | 636.9 | P | 742.9 | P |
| | | clone_end = 3/gb = AW046723/gi = 5907292/ug = Mm 45233/len = 220/STRA = for | | | | | | | |
| 2 | 100099_at | Cluster Incl Z14132 Sphingomyelin phosphodiesterase 1, acid lysosomal/cds = (0,1883)/gb = Z14132/gi = 475954/ | P | 1138.8 | P | 1244.8 | P | 1828.6 | P |
| | | ug = Mm 4628/len = 1884/STRA = for | | | | | | | |
| 2 | 100133_at | Cluster Incl M27266 Fyn proto-oncogene/cds = (225,1829)/gb = M27266/gi = 193357/ug = Mm.4848/len = 2299/STRA = for | P | 230.9 | P | 281.3 | P | 372.2 | P |
| 2 | 100155_at | Cluster Incl L57509 Cell adhesion kinase/cds = (106,2841)/gb = L57509/gi = 1161062/ug = Mm.5021/len = 3674/STRA = for | P | 422.8 | P | 555.6 | P | 746.5 | P |
| 2 | 100561_at | Cluster Incl AW209098: un99g09 x1 Mus musculus cDNA, 3 end/clone = IMAGE-2582080/clone_end = 3/gb = AW209098/ | P | 738.4 | P | 913.8 | P | 791 | P |
| | | gi = 6515038/ug = Mm 56685/len = 486/STRA = for | | | | | | | |
| 2 | 100605_at | Cluster Incl M81086 Mouse skeletal muscle beta tropomyosin mRNA, complete cds/cds = (34,888)/gb = M81086/gi202162/ | P | 706.4 | P | 1207.1 | P | 1172.8 | P |
| | | ug = Mm 646/len = 2097/STRA = for | | | | | | | |
| 2 | 100635_at | Cluster Incl L20294 Mus musculus GTP-binding protein (mSara) homologue mRNA, complete cds/cds = (84,680)/ | P | 1596.9 | P | 1610.3 | P | 2034.4 | P |
| | | gb = L20294/gi = 436563/ug = Mm 6698/len = 696/STRA = for | | | | | | | |
| 2 | 101028_l_at | Cluster Incl M15501 Actin, alpha, cardiac/cds = (0,1127)/gb = M15501/gi = 191653/ug = Mm 686/len = 1226/STRA = for | P | 1656.8 | P | 1076.6 | P | 4511.2 | P |
| 2 | 101030_at | Cluster Incl X99963: M. musculus rhoB gene/cds = (0,590)/gb = X99963/gi = 1491740/ug = Mm 687/len = 591/STRA = for | P | 1471.4 | P | 1776.3 | P | 1645.2 | P |
| 2 | 101069_g_at | Cluster Incl AA656621: vr49a10 s1 Mus musculus cDNA, 5 end/clone = IMAGE-1123962/clone_end = 5/gb = AA656621/ | P | 428.2 | P | 401.6 | P | 1466.8 | P |
| | | gi = 2592775/ug = Mm 7198/len = 335/STRA = for | | | | | | | |
| 2 | 101070_at | Cluster Incl AW125438 UI-M-BH2 3-aqh-g-08-0-UI s1 Mus musculus cDNA, 3 end/clone = UI-M-BH2 3-aqh-g-08-0-UI/ | P | 320.4 | P | 354.2 | P | 462.2 | P |
| | | clone_end = 3/gb = AW125438/gi = 6100968/ug = Mm 7198/len = 442/STRA = for | | | | | | | |
| 2 | 101502_at | Cluster Incl X89749.M. musculus mRNA for mTGIF protein/cds = (279,1097)/gb = X89749/gi = 1495713/ug = Mm.8155 | P | 385.9 | P | 351.2 | P | 413.9 | P |
| | | len = 1186/STRA = for | | | | | | | |
| 2 | 101554_at | Cluster Incl U57524 Mus musculus I kappa B alpha gene, exons 2-6, partial cds/cds = (0,717)/gb = U57524/gi = 4097334/ | P | 678.8 | P | 483 | P | 1238.2 | P |
| | | ug = Mm 8884/len = 815/STRA = for | | | | | | | |
| 2 | 101977_at | Cluster Incl AW121873 UI-M-BH2 3-aoi-d-02-0-UI.s1 Mus musculus cDNA, 3 end/clone = UI-M-BH2 3-aoi-d-02-0-UI/ | P | 902.2 | P | 879.4 | P | 1003.1 | P |
| | | clone_end = 3/gb = AW121873/gi = 6097247/ug = Mm 95709/len = 491/STRA = for | | | | | | | |
| 2 | 102363_r_at | Cluster Incl U20735 Mus musculus transcription factor junB (junB) gene, 5 region and complete cds/cds = (294,1328)/ | P | 345 | P | 476.8 | P | 426.6 | P |
| | | gb = U20735/gi = 965003/ug = Mm.1167/len = 1786/STRA = for | | | | | | | |
| 2 | 102751_at | Cluster Incl M32745. Transforming growth factor, beta 3/cds = (610,1842)/gb = M32745/gi = 201949/ug = Mm 1291/len = 2879/ | P | 417.2 | P | 485.2 | P | 480.1 | P |
| | | STRA = for | | | | | | | |
| 2 | 102792_at | Cluster Incl U55040 Mus musculus uracil-DNA glycosylase (ung) gene, exon 1, partial cds/cds = (27,311)/gb = U55040/ | P | 971.1 | P | 947.1 | P | 786.6 | P |
| | | gi-1762315/ug = Mm 1393/len = 312/STRA = for | | | | | | | |
| 2 | 102991_s_at | Cluster Incl AF100956 Mus musculus major histocompatibility locus class II region; Fas-binding protein Daxx (DAXX) gene, | P | 193.4 | P | 174 | P | 246.2 | P |
| | | partial cds; Bing1 (BING1), tapasin (tapasin), RalGDS-like factor (RLF), KE2 (KE2), BING4 (BING4), beta1,3-galactosyl | | | | | | | |
| | | transferase (be/STRA = for | | | | | | | |
| 2 | 102996_at | Cluster Incl U80227: Mus musculus ELL homolog mRNA, complete cds/cds = (5,1813)/gb = U80227/gi = 1947072/ | P | 347.1 | P | 280.4 | P | 460.2 | P |
| | | ug = Mm. 1552/len = 3023/STRA = for | | | | | | | |
| 2 | 103031_g_at | Cluster Incl L31397: Dynamin/cds = (73,2658)/gb = L31397/gi = 487858/ug = Mm.16080/len = 3924/STRA = rev | P | 587.4 | P | 637.5 | P | 649.1 | P |
| 2 | 103069_at | Cluster Incl AI853682 UI-M-BH0-ajq-h-03-0-UI s1 Mus musculus cDNA, 3 end/clone = UI-M-BH0-ajq-h-03-0-UI/ | P | 264.1 | P | 230.4 | P | 291.4 | P |
| | | clone_end = 3/gb = AI853682/gi = 5497628/ug = Mm 16813/len = 425/STRA = for | | | | | | | |
| 2 | 103098_at | Cluster Incl AW045765 UI-M-BH1-akt-c-06-0-UI s1 Mus musculus cDNA, 3 end/clone = UI-M-BH1-akt-c-06-0-UI/ | P | 657.1 | P | 847.3 | P | 733.2 | P |
| | | clone_end = 3/gb = AW045765/gi = 5906294/ug = Mm. 17533/len = 338/STRA = for | | | | | | | |
| 2 | 103343_at | Cluster Incl AI845815 UI-M-AO1-aeg-b-02-0-UI S1 Mus musculus cDNA, 3 end/clone = UI-M-AO1-aeg-b-02-0-UI/ | P | 251.8 | P | 250.2 | P | 394.7 | P |
| | | clone_end = 3/gb = AI845815/gi = 5489721/ug = Mm 18228/len = 367/STRA = for | | | | | | | |
| 2 | 103636_at | Cluster Incl AI840971 UI-M-AK0-adk-e-02-0-UI s1 Mus musculus cDNA, 3 end/clone = UI-M-AK0-adk-e-02-o-UI/ | P | 498.7 | P | 520.3 | P | 529.4 | P |
| | | clone_end = 3/gb = AI840971/gi = 5475184/ug = Mm 20324/len = 389/STRA = for | | | | | | | |
| 2 | 104094_at | Cluster Incl Y08361 M musculus mRNA for RIL protein/cds = (36,1028)/gb = Y08361/gi = 1585268/ug = Mm.21830/len = 1135/ | P | 188.8 | P | 193.7 | P | 269.5 | P |
| | | STRA = for | | | | | | | |
| 2 | 104116_at | Cluster Incl AW124049 UI-M-BH2 1-apn-e-03-0-UI s1 Mus musculus cDNA, 3 end/clone = UI-M-BH2 1-apn-e-03-0-UI/ | P | 211.2 | P | 358.3 | P | 560.5 | P |
| | | clone_end = 3/gb = AW124049/gi = 6099579/ug = Mm 21965/len = 382/STRA = for | | | | | | | |
| 2 | 104149_at | Cluster Incl AI642048 ub75b05.x1 Mus musculus cDNA, 3 end/clone = IMAGE-1383537/clone_end = 3/gb = AI642048/ | P | 595.3 | P | 407.7 | P | 799.9 | P |
| | | gi = 4720523/ug = Mm 22188/len = 434/STRA = for | | | | | | | |
| 2 | 104286_at | Cluster Incl AA795541.vq96e09 r1 Mus musculus cDNA, 5 end/clone = IMAGE-1110184/clone_end = 5/gb = AA795541/ | P | 502.8 | P | 413.8 | P | 913 | P |
| | | gi = 2858496/ug = Mm 22260/len = 393/STRA = for | | | | | | | |
| 2 | 104302_f_at | Cluster Incl AI851085 UI-M-BH0-akg-b-04-0-UI.s1 Mus musculus cDNA,3 end/clone = UI-M-BH0-akg-b-04-0-UI/ | P | 446.4 | P | 549.5 | P | 653.7 | P |
| | | clone_end = 3/gb = AI851085/gi = 5494991/ug = Mm 22353/len = 486/STRA = for | | | | | | | |
| 2 | 104352_at | Cluster Incl AI838366 UI-M-AI0-aak-f-10-0-UI.s2 Mus musculus cDNA, 3 end/clone = UI-M-AI0-aak-f-10-0-UI/clone_end = 3/ | P | 988.9 | P | 1067.1 | P | 1043.2 | P |
| | | gb = AI838366/gi = 5472579/ug = Mm 22572/len = 319/STRA = for | | | | | | | |
| 2 | 104533_at | Cluster Incl AA764261 W49f08.r1 Mus musculus cDNA, 5 end/clone = IMAGE-1225767/clone_end = 5/gb = AA764261/ | P | 1066.5 | P | 928.4 | P | 964.9 | P |
| | | gi = 2811783/ug = Mm 2322/len = 320/STRA = for | | | | | | | |
| 2 | 104545_at | Cluster Incl U51866 Mus musculus casein kinase II alpha subunit mRNA, complete cds/cds = (203,1378)/gb = U51866/ | P | 204.4 | P | 289.8 | P | 507.4 | P |
| | | gi = 1262299/ug = Mm 23692/len = 1552/STRA = for | | | | | | | |
| 2 | 104598_at | Cluster Incl X61940: Mouse mRNA for a growth factor-inducible immediate early gene (3CH134)/cds = (139,1242)/ | P | 927.8 | P | 638.5 | P | 1415.7 | P |
| | | gb = X61940/gi = 49735/ug = Mm 2404/len = 1933/STRA = for | | | | | | | |
| 2 | 104711_at | Cluster Incl AW122109 UI-M-BH2 2-aor-a-06-0-UI s1 Mus musculus cDNA, 3 end/clone = UI-M-BH2 2-aor-a-06-0-UI/ | P | 540 .6 | P | 515.5 | P | 435.5 | P |
| | | clone_end = 3/gb = AW122109/gi = 6097524/ug = Mm 24437/len = 352/STRA = for | | | | | | | |
| 2 | 94378_at | Cluster Incl U94828 Mus musculus retinally abundant regulator of G-protein signaling mRGS-r (RGS-r) mRNA, complete | P | 335.6 | P | 383.5 | P | 749.4 | P |
| | | cds/cds = (109,714)/gb = U94828/gi = 2605641/ug = Mm 2555/len = 2250/STRA = for | | | | | | | |
| 2 | 94957_at | Cluster Incl AW121154: UI-M-BH2 3-aoh-f-12-0-UI s1 Mus musculus cDNA, 3 end/clone = UI-M-BH2 3-aoh-f-12-0-UI/ | P | 206 .7 | P | 146.7 | P | 168.5 | P |
| | | clone_end = 3/gb = AW121154/gi = 6096487/ug = Mm 27167/len = 421/STRA = for | | | | | | | |
| 2 | 96128_at | Cluster Incl X69026: PCTAIRE-motif protein kinase 3/cds = (318,1673)/gb = X69026/gi = 53612/ug = Mm 28130/len = 2997/ | P | 357.2 | P | 352 | P | 362.2 | P |
| | | STRA = for | | | | | | | |
| 2 | 96132_at | Cluster Incl AB023957 Mus musculus EIG 180 mRNA for ethanol induced gene product, complete cds/cds = (140,481)/ | P | 1123.4 | P | 1254.4 | P | 1309.1 | P |
| | | gb = AB023957/gi = 6468203/ug = Mm.28143/len = 728/STRA = for | | | | | | | |
| 2 | 96147_at | Cluster Incl AW125070 UI-M-BH2 1-apx-d-08-0-UI s1 Mus musculus cDNA, 3 end/clone = UI-M-BH2 1-apx-d-08-0-UI/ | P | 231.2 | P | 336.3 | P | 327.5 | P |
| | | clone_end = 3/gb = AW125070/gi = 6100600/ug = Mm 28230/len = 438/STRA = for | | | | | | | |
| 2 | 96723_f _at | Cluster Incl AW122911 UI-M-BH2-1-apa-f-09-0-UI s1 Mus musculus cDNA, 3 end/clone = UI-M-BH2 1-apa-f-09-0-UI/ | P | 539.9 | P | 756.6 | P | 713.7 | P |
| | | clone_end = 3/gb = AW122911/gi = 6098441/ug = Mm 28829/len = 366/STRA = for | | | | | | | |
| 2 | 96742_at | Cluster Incl AA717826 vt98g09 r1 Mus musculus cDNA, 5 end/clone = IMAGE-1179232/clone_end = 5/gb = AA717826/ | P | 596.7 | P | 884.8 | P | 860.7 | P |
| | | gi = 2730100/ug = Mm 28935/len = 255/STRA = for | | | | | | | |
| 2 | 96797_s_at | Cluster Incl AW122681 UI-M-BH2 2-aot-a-02-0-UI s1 Mus musculus cDNA, 3 end/clone = UI-M-BH2 2-aot-a-02-0-UI/ | P | 747.8 | P | 735.9 | P | 916 | P |
| | | clone_end = 3/gb = AW122681/gi = 6098211/ug = Mm 29170/len = 349/STRA = for | | | | | | | |
| 2 | 96813_f_at | Cluster Incl AI852973 UI-M-BH0-aiz-g-09-0-UI s1 Mus musculus cDNA, 3 end/clone = UI-M-BH0-aiz-g-09-0-UI/ | P | 704.1 | P | 667.5 | P | 914.8 | P |
| | | clone_end = 3/gb = AI852973/gi = 5496879/ug = Mm 29297/len = 375/STRA = for | | | | | | | |
| 2 | 97347_at | Cluster Incl AA838868 vw78c04.r1 Mus musculus cDNA, 5 end/clone = IMAGE-1261062/clone_end = 5/gb = AA838868/ | P | 457.3 | P | 449.5 | P | 680 | P |
| | | gi = 2914963/ug = Mm 29618/len = 493/STRA = rev | | | | | | | |
| 2 | 97349_at | Cluster Incl AA727410 vu99g08 r1 Mus museulus cDNA, 5 end/clone = IMAGE-1210334/clone_end = 5/gb = AA727410/ | P | 738.4 | P | 758.3 | P | 1405.1 | P |
| | | gi = 2745117/ug = Mm.2962/len = 506/STRA = for | | | | | | | |
| 2 | 97366_at | Cluster Incl AI851024 UI-M-BH0-ajv-d-07-o-UI s1 Mus musculus cDNA, 3 end/clone = UI-M-BH0-ajv-d-07-0-UI/ | P | 379.4 | P | 357 | P | 496.4 | P |
| | | clone_end = 3/gb = AI851024/gi = 5494930/ug = Mm.29689/len = 132/STRA = for | | | | | | | |
| 2 | 97370_at | Cluster Incl AI552922 vg73I08 y1 Mus musculus cDNA, 5 end/clone = IMAGE-871623/clone_end = 5/gb = AI552922/ | P | 311.3 | P | 270 | P | 303.2 | P |
| | | gi = 4485285/ug = Mm 29694/len = 222/STRA = fof | | | | | | | |
| 2 | 97405_at | Cluster Incl M28489: Mus musculus (dona Mu6a) ribosomal pratotein S6 kinase (rsk) mRNA, complete cds/cds = (151,2325)/ | P | 604.2 | P | 495.1 | P | 467.3 | P |
| | | qb = M28489/gi = 556321/ug = Mm 29910/len = 3081/STRA = for | | | | | | | |
| 2 | 97423_at | Cluster Incl AI839819 UI-M-AN0-acq-c-03-0-UI s1 Mus musculus cDNA, 3 end/clone = UI-M-AN0-acq-c-03-0-UI/ | P | 511.8 | P | 642.7 | P | 415.1 | P |
| | | clone_end = 3/gb = AI839819/gi = 5473997/ug = Mm.30006/len = 305/STRA = rev | | | | | | | |
| 2 | 97429_at | Cluster Incl AW048113 UI-M-BH1-alq-d-12-0-UI s1 Mus musculus cDNA, 3 end/clone = UI-M-BH1-alq-d-12-0-UI/ | P | 396.1 | P | 394.4 | P | 573 | P |
| | | clone_end = 3/gb = W048113/gi = 5908642/ug = Mm.30079/len = 395/STRA = for | | | | | | | |
| 2 | 97921_at | Cluster Incl AI846279 UI-M-AK1-aez-e-04-0-UI s1 Mus musculus cDNA, 3 end/clone = UI-M-AK1-aez-e-04-0-UI/ | P | 1064.6 | P | 764.8 | P | 998.6 | P |
| | | clone_end = 3/gb = AI846279/gi = 5490185/ug = Mm 30145/len = 409/STRA = for | | | | | | | |
| 2 | 97943_at | Cluster Incl AI747133 ui12a02.x1 Mus musculus cDNA, 3 end/clone = IMAGE-2076554/clone_end = 3/gb = AI747133/ | P | 809.2 | P | 659.2 | P | 944.1 | P |
| | | gi = 5125397/ug = Mm 30290/len = 560/STRA = for | | | | | | | |
| 2 | 97982_at | Cluster Incl AA260139 va37a08 r1 Mus musculus cDNA, 5 end/clone = IMAGE-733526/clone_end = 5/gb = AA260139/ | P | 318.9 | P | 287.2 | P | 369 | P |
| | | gi = 1896624/ug = Mm 3127/len = 303/STRA = for | | | | | | | |
| 2 | 98999_at | Cluster Incl AA606587.vm86b01 r1 Mus musculus cDNA, 5 end/clone = IMAGE-1005097/clone_end = 5/gb = AA606587/ | P | 277.1 | P | 245.9 | P | 247.2 | P |
| | | gi = 245521/ug = Mm 38151/len = 559/STRA = for | | | | | | | |
| 2 | 99042_s_at | Cluster Incl U66918: Short stature homeobox 2/cds = (215,1210)/gb = U66918/gi = 1616757/ug = Mm.39093/len = 1603/ | P | 294.9 | P | 384.5 | P | 426.6 | P |
| | | STRA = for | | | | | | | |
| 2 | 99052_at | Cluster Incl D76432: Transcriptrion factor 8/cds = (19,3372)/gb = D76432/gi = 1027499/ug = Mm 3929/len = 5214/STRA = rev | P | 326.1 | P | 312.3 | P | 342.5 | P |
| 2 | 99475_at | Cluster Incl UB8327: Mus musculus suppressor of cytokine signalling-2 (SOCS-2) mRNA, complete cds/cds = (222,818)/ | P | 366.4 | P | 301.6 | P | 516.8 | P |
| | | gb = U88327/gi = 2245385/ug = Mm.4132/len = 1121/STRA = for | | | | | | | |
| 2 | 99978_s_at | Cluster Incl D83073 Mouse mRNA for p38b, complete cds/cds = (229,1311)/gb = D83073/gi = 1181606/ug = Mm 4437/ | P | 1080.2 | P | 923.6 | P | 1250.2 | P |
| | | len = 3352/STRA = for | | | | | | | |
| 2 | 100005_at | Cluster Incl X92346 Tnf receptor associated factor 4/cds = (130,1542)/gb = X92346/gi = 1041445/ug = Mm 4521/len:2043/ | P | 603.9 | P | 481.2 | P | 688.8 | P |
| | | STRA = for | | | | | | | |
| 2 | 100017_at | Cluster Incl U68267: Mus musculus myosin binding protein H (MyBP-H) mRNA, complete cds/cds = (50,1501)/gb = U68267/ | P | 374.7 | P | 370.6 | P | 558.7 | P |
| | | gi = 1546812/ug = Mm 4562/len = 1850/STRA = for | | | | | | | |
| 2 | 100022_at | Cluster Incl D89613: Cytokine inducible SH2-containing protein/cds = UNKNOWN/gb = D89613/gi = 2554633/ug = Mm 4592/ | P | 699.4 | P | 982.2 | P | 771.1 | P |
| | | len = 1010/STRA = for | | | | | | | |
| 2 | 100024_at | Cluster Incl AI641895vq44e08 y1 Mus musculus cDNA, 5 end/clone = IMAGE-1097126/clone_end = 5/gb = AI641895/ | P | 425.1 | P | 446.2 | P | 600.2 | P |
| | | gi = 4720370/ug = Mm 46014/len = 413/STRA = for | | | | | | | |
| 2 | 100407_at | Cluster Incl L38580 Galanin/cds = (0,374)/gb = L38580/gi = 927115/ug = Mm.4655/len = 553/STRA = for | P | 380.3 | P | 441.6 | P | 507.4 | P |
| 2 | 100441_s_at | Cluster Incl X69064: Ankyrin 1, erythroid/cds = (0,731)/gb = X69064/gi = 311818/ug = Mm 4789/len = 1704/STRA = for | P | 88.2 | P | 123.3 | P | 143.6 | P |
| 2 | 100488_at | Cluster Incl M24119: Acid beta glucosidase/cds = (86,1633)/gb = M24119/gi = 193449/ug = Mm.5031/len = 1684/STRA = for | P | 574.3 | P | 675.5 | P | 839.1 | P |
| 2 | 100877_at | Cluster Incl AI194274 ul14c07 r1 Mus musculus cDNA, 5 end/clone = IMAGE-1851276/clone_end = 5/gb = AI194274/ | P | 614.2 | P | 541 | P | 1181.2 | P |
| | | gi = 3745481/ug = Mm 53/len = 480/STRA = for | | | | | | | |
| 2 | 100899_s_at | Cluster Incl AI265210 ul97a10.x1 Mus musculus cDNA, 3 end/clone = IMAGE-1890330/clone_end = 3/gb = AI265210/ | P | 532.4 | P | 512.7 | P | 693 | P |
| | | gi = 3883368/ug = Mm 57157/len = 485/STRA = for | | | | | | | |
| 2 | 100928_at | Cluster Incl X75285 Fibulin 2/cds = (50,3715)/gb = X75285/gi = 437046/ug = Mm 6120/len = 4377/STRA = for | P | 834.6 | P | 896.8 | P | 1451.6 | P |
| 2 | 100960_g_at | Cluster Incl X99921 M musculus mRNA for S100 calcium-binding protein A13/cds = (60,356)/gb = X99921/gi = 1694840/ | P | 797.9 | P | 720.5 | P | 811.9 | P |
| | | ug = Mm 6523/len = 440/STRA = for | | | | | | | |
| 2 | 101357_at | Cluster Incl X14971 Mouse mRNA for alpha-adaptin (A)/cds = (205,3138)/gb = X14971/gi = 49877/ug = Mm.6877/len = 3496/ | P | 400.1 | P | 360.4 | P | 512.4 | P |
| | | STRA = for | | | | | | | |
| 2 | 102209_at | Cluster Incl AF087434.Mus musculus transcription factor NF-ATc isoform a (NF-ATca) mRNA, complete cds/cds = (0,2153/) | P | 85.7 | P | 195.9 | P | 145.8 | P |
| | | gb = AF087434/gi = 3643194/ug = Mm 10085/len = 3469/STRA = for | | | | | | | |
| 2 | 102213_at | Cluster Incl AF051945 Mus musculus Xin mRNA, complete cds/cds = (140,5173)/gb = AF051945/gi = 2970645/ | P | 166.1 | P | 273.4 | P | 323.2 | P |
| | | ug = Mm 10117/len = 5800/STRA = for | | | | | | | |
| 2 | 102216_at | Cluster Incl Y14334 Arachidonale 12-lipoxygenase, pseudogene 2/cds = (175,2280)/gb = Y14334/gi = 3043452/ | P | 389.1 | P | 220.9 | P | 742.1 | P |
| | | ug = Mm 10143/len = 2347/STRA = for | | | | | | | |
| 2 | 102283_at | Clustar Incl U05245 T-cell lymphoma invasion and metastasis 1/cds = (506,5281)/gb = U05245/gi = 497638/ug = Mm.1211/ | P | 656.5 | P | 576.3 | P | 668.9 | P |
| | | 1en = 7292/STRA = for | | | | | | | |
| 2 | 102683_at | Cluster Incl U76009 Zinc transporter 3/cds = (0,1025)/gb = U76009/gi = 1763379/ug = Mm.1396/len = 1026/STRA = for | P | 1228.7 | P | 1206.4 | P | 1741.7 | P |
| 2 | 102737_at | Cluster Incl U35233 Endothelin 1/cds = (30,638)/gb = U35233/gi = 1244501/ug = Mm 14543/len = 1770/STRA = for | P | 233.1 | P | 165.4 | P | 349.5 | P |
| 2 | 102955_at | Cluster Incl U83148 Mus musculus NFIL3/E4BP4 transaction factor mRNA, complete cds/cds = (0,1388)/gb = U83148/ | P | 137.9 | P | 132.1 | P | 174.3 | P |
| | | gi = 2076877/ug = Mm 1755/len = 1389/STRA = for | | | | | | | |
| 2 | 103215_g_at | Cluster Incl AW048883 UI-M-BH1-amf-f-01-0-UI.s1 Mus musculus cDNA, 3 end/clone = UI-M-BH1-amf-l-01-0-UI/ | P | 627.9 | P | 683 | P | 995.4 | P |
| | | clone_end = 3/gb = AW048883/gi = 5909412/ug = Mm 1980/len = 489/STRA = for | | | | | | | |
| 2 | 103504_at | Cluster Incl AI837107.UI-M-AK0-adc-d-11-0-UI s1 Mus muaculus cDNA, 3 end/clone = UI-M-AK0-adc-d-11-0-UI/ | P | 426.9 | P | 418.6 | P | 407 | P |
| | | clone_end = 3/gb = AI637107/gi = 5471320/ug = Mm 22438/len = 460/STRA = for | | | | | | | |
| 2 | 103736_at | Cluster Incl AI837786: UI-M-AK0-adl-e-02-0-UI s1 Mus musculus cDNA, 3 end/clone = UI-M-AK0-adl-e-02-0-UI/ | P | 289.2 | P | 334.3 | P | 419.4 | P |
| | | clone_end = 3/gb = AI837786/gi = 5471999/ug = Mm24385/len = 412/STRA = for | | | | | | | |
| 2 | 104000_at | Cluster lncl AI181346.ub83d07.r1 Mus musculus cDNA, 5 end/clone = IMAGE-1395085/clone_end-5/gb = AM81346/ | P | 1341.6 | P | 1097.4 | P | 1456.6 | P |
| | | gi = 3731984/ug = Mm 26580/len = 474/STRA = for | | | | | | | |
| 2 | 104155_f_at | Cluster Incl U19118 Mus musculus transcription factor LRG-21 mRNA, complete cds/cds = (167,712)/gb = U19118/ | P | 269.6 | P | 240.8 | P | 319.1 | P |
| | | gi = 633751/ug = .Mm2706/len = 1897/STRA = for | | | | | | | |
| 2 | 104435_at | Cluster Incl AI844072: vu70c06 x1 Mus musculua cDNA, 3 end/clone = IMAGE-1196746/clone_end = 3/gb = AI644072/ | P | 1038.1 | P | 895.3 | P | 1276 | P |
| | | gi = 4722547/ug = Mm 29257/len = 460/STRA = for | | | | | | | |
| 2 | 104477_at | Cluster Incl AW047643: UI-M-BH1-alo-a-02-0-Ul.s1 Mus musculus cDNA. 3 end/clone = UI-M-BH1-alo-a-02-0-UI/ | P | 144.6 | P | 119.6 | P | 896.4 | P |
| | | clone_end = 3/gb = AW047643/gl = 5908172/ug = Mm 29940/len = 375/STRA = for | | | | | | | |
| 2 | 92185_at | Cluster Incl AI846023.UI-M-AP1-agl-g-02-0-Ul s1 Mus musculus cDNA, 3 end/clone = UI-M-AP1-agl-g-02-0-UI/ | P | 356 | P | 654.5 | P | 484.9 | P |
| | | clone_end = 3/gb = AI846023/gi = 5489929/ug = Mm 3388/len = 482/STRA = for | | | | | | | |
| 2 | 92216_at | Cluster Incl AF015260 Mus musculus Smad7 mRNA, complele cds/cds = (0,1280)/gb = AF015260/gi = 2460039/ | P | 188.3 | P | 241.7 | P | 194 | P |
| | | ug = Mm 34407/len = 1281/STRA = for | | | | | | | |
| 2 | 92220_s_at | Cluster Incl U60884 Mus musculus SH3P9 mRNA, complele cds/cds = (178,1482)/gb = U60884/gi = 1438562/ug = Mm.34445/ | P | 428.6 | P | 455.2 | P | 932.5 | P |
| | | len = 1531/STRA = for | | | | | | | |
| 2 | 92325_at | Cluster Incl AI326889 mj61doe.x1 Mus musculus cDNA, 3 end/clone = IMAGE-480587/clone_end = 3/gb = AI326889/ | P | 212.7 | P | 304.6 | P | 286.3 | P |
| | | gi = 4061318/ug = Mm 38494/len = 494/STRA = for | | | | | | | |
| 2 | 92352_at | Cluster Incl AF108021.Mus musculus lysophospholipid receptor B3 (Ipb3) gene, complete cds/cds = (0,1136)/gb = AF108021/ | P | 424 | P | 577.3 | P | 543 | P |
| | | gi = 4324652/ug = Mm 39366/len.1137/STRA = for | | | | | | | |
| 2 | 92376_at | Cluster Incl U71205 Mus musculus ril mRNA, complete cds/cds = (80,739)/gb = U71205/gi = 1656004/ug = Mm.4009/ | P | 817.5 | P | 649.8 | P | 817.5 | P |
| | | len = 1079/STRA = for | | | | | | | |
| 2 | 92484_at | Cluster Incl Y15907 Mus musculus mRNA for myc-intron-binding proteln-1/cds = (84,7376)/gb = Y15907/gi = 2706548/ | P | 100.6 | P | 97.4 | P | 136.9 | P |
| | | ug = Mm 42157/len = 8095/STRA = rev | | | | | | | |
| 2 | 92730_at | Cluster Incl L07284 Heparin binding epidermal growth factor-like growth factor/cds = (225,851)/gb = L07264/gl = 192999/ | P | 150.6 | P | 11.9 | P | 427.9 | P |
| | | ug = Mm 4661/len1000/STR = for | | | | | | | |
| 2 | 92968_at | Cluster Incl D87902: Mouse mRNA for ARF5, complete cds/cds = (49,591)/gb = D87902/gi = 1565214/ug = Mm.4996/len = 933/ | P | 930.1 | P | 1059.9 | P | 1307.4 | P |
| | | STRA = for | | | | | | | |
| 2 | 93187_at | Cluster Incl AW048347: UI-M-BH1-akk-e-03-0-Ul.s1 Mus musculus cDNA, 3 end/clone = Ul-M-BH1-akk-e-03-0-Ul/ | P | 538.4 | P | 544.3 | P | 681.1 | P |
| | | clone end = 3/gb = AW048347/gl = 5908876/ug = Mm 5510/len = 458/STRA = for | | | | | | | |
| 2 | 93197_at | Cluster Incl AF109905 Mus musculus major histocompatibility locus class III regions Hsc70t gene, partial cds; smRNP, | P | 1131.5 | P | 1273.4 | P | 2032.5 | P |
| | | G7A, NG23, MutS homolog, CLCP, NG24, NG25, and NG26 genes, complete cds, and unknown genes/cds = (0,380)/ | | | | | | | |
| | | gb = AF 109905/gi = 3986751/ug = Mm 56/STRA = for | | | | | | | |
| 2 | 93434_at | Cluster Incl AF026074 Mus musculus sulfotransferase-related protein (SULT-X1) mRNA, complete cds/cds = (88,963)/ | P | 134.7 | P | 89.8 | P | 159.4 | P |
| | | gb = AF026074/gi = 2570901/ug = Mm 6562/len = 1173/STRA = for | | | | | | | |
| 2 | 93852_at | Cluster Incl AW045443: UI-M-BH1-akr-f-12-0-UI s1 Mus musculus cDNA, 3 end/clone = UI-M-BM1-akr-f-12-0-UI/ | P | 569.2 | P | 603.4 | P | 549.5 | P |
| | | clone_end = 3/gb = AW045443/gi = 5906012/ug = Mm 87279/len = 292/STRA = for | | | | | | | |
| 2 | 93922_g_at | Cluster Incl AI573601-ud46a12.y1 Mus musculus cDNA, 5 end/clone = IMAGE-1448926/clone_end = 5/gb = AI573601/ | P | 487.6 | P | 551 | P | 759.2 | P |
| | | gi = 4536975/ug = Mm 89855/len = 535/STRA = for | | | | | | | |
| 2 | 93965_r_at | Cluster Incl AF038995 Mus musculus putative RNA helicase RCK mRNA, partial cds/cds = (0,1359)/gb = AF038995/ | P | 342.5 | P | 724.7 | P | 390.8 | P |
| | | gi = 2745893/ug = Mm 97095/len = 1360/STRA = for | | | | | | | |
| 2 | 95286_at | Cluster Incl D14077 Clusterin/cds = (91,1437)/gb = D14077/gi = 220601/ug = Mm 18700/len = 1663/STRA = for | P | 561.5 | P | 767.2 | P | 874.6 | P |
| 2 | 95345_at | Cluster Incl AJ012160 Mus musculus 5T4 oncofetal trophoblast glycoprotein gene/cds = (328,1608)/gb = AJ012160/ | P | 267 | P | 250.2 | P | 284.8 | P |
| | | gi = 3805948/ug = Mm.20864/len = 2329/STRA = for | | | | | | | |
| 2 | 95383_at | Cluster Incl AI415418 mc4Be08 x1 Mus musculus cDNA, 3 end/clone = IMAGE-351782/clone_end = 3/gb = AI415418/ | P | 664.8 | P | 772.3 | P | 1438.1 | P |
| | | gi = 4258922/ug = Mm 23082/len = 455/STRA = rev | | | | | | | |
| 2 | 95989_at | Cluster Incl AI448172 mo64107 x1 Mus musculus cDNA, 3 end/clone = IMAGE-558373/clone_end = 3/gb = AI448172/ | P | 681.4 | P | 651 | P | 572.1 | P |
| | | gi = 4309126/ug = Mm 25049/len = 373/STRA = for | | | | | | | |
| 2 | 97114_at | Cluster Incl U57999 Prosaposin/cds = (25,1698)/gb = U57999/gi = 1381581/ug = Mm 3363/len = 1961/STRA = for | P | 1554.6 | P | 1382.3 | P | 2089.1 | P |
| 2 | 97132_at | Cluster Incl AA189890 mu55b11 r1 Mus musculus cDNA,5 end/clone = IMAGE-643293/clone_end = 5/gb = AA189890/ | P | 786.7 | P | 901.5 | P | 906.4 | P |
| | | gi = 1776532/ug = Mm 34876/len = 344/STRA = rev | | | | | | | |
| 2 | 97165_r_at | Cluster Incl C77278 C77278 Mus musculus cDNA, 3 end/clone = J0028D08/clone_end = 3/gb = C77278/gi = 2517608/ | P | 137.9 | P | 257.4 | P | 244.7 | P |
| | | ug = Mm 35523/len = 591/STRA = rev | | | | | | | |
| 2 | 97193_at | Cluster Incl AA575098 vh82h08 r1 Mus musculus cDNA, 3 end/clone = IMAGE-893535/clone_end = 3/gb = AA575098/ | P | 258.9 | P | 145.7 | P | 179.5 | P |
| | | gi = 2349724/ug = Mm 36566/len = 336/STRA = rev | | | | | | | |
| 2 | 99367_at | Cluster Incl AA434661 ve16g03 r1 Mus musculus cDNA, 5 end/clone = IMAGE-818356/clone_end = 5/gb = AA434661/ | P | 26.3 | P | 39.3 | P | 96.4 | P |
| | | gi = 2139575/ug = Mm 56829/len = 320/STRA = for | | | | | | | |
| 2 | 99399_at | Cluster Incl U89486 Mus musculus agouti-related protein (Agrp) gene, complete cds/cds = (0,395)/gb = U89486/gi = 2351716/ | P | 302.5 | P | 471.6 | P | 386.7 | P |
| | | ug = Mm 56995/len = 396/STRA = for | | | | | | | |
| 2 | 99851_at | Cluster Incl AA172988 ms58e02 r1 Mus musculus cDNA, 5 end/clone = IMAGE-615770/clone_end = 5/gb = AA172988/ | P | 708.1 | P | 824.8 | P | 1003.9 | P |
| | | gi = 1752041/ug = Mm 6436/len = 344/STRA = for | | | | | | | |
| 2 | 95215_f_at | Cluster Incl D50527.Mouse mRNA for TI-225, complete cds/cds = (37,417)/gb = D50527/gi = 1167509/ug = Mm 331/len = 498/ | P | 2633.9 | P | 3319.5 | P | 316.5 | P |
| | | STRA = for | | | | | | | |
| 2 | 92830_s_at | Cluster Incl X14678 Zinc finger protein 36/cds = (756,1307)/gb = X14678/gi = 54803/ug = Mm.1298/len = 2282/STRA = for | P | 452 | P | 266.9 | A | 373.9 | P |
| 2 | 92838_at | Cluster Incl L33726 Fascin 1/cds = (78,1559)/gb = L33726/gi = 497774/ug = Mm.13194/len = 2634/STRA = for | P | 1074.1 | P | 1255.1 | P | 944.2 | A |
| 2 | 92874_f_at | Cluster Incl AF071316 Mus musculus COP9 complex subunit 7a (COPS7a) mRNA, complete cds/cds = (158,985)/ | P | 370.7 | P | 323.6 | P | 621.3 | A |
| | | gb = AF071316/gi = 3309173/ug = Mm.1444/len = 1779/STRA = for | | | | | | | |
| 2 | 94063_at | Cluster Incl X85991 M musculus mRNA for semaphorin B/cds = (65,2347)/gb = X85991/gi = 854325/ug = Mm.22061/ | A | 378.5 | P | 425.5 | P | 662.2 | P |
| | | len = 3046/STRA = for | | | | | | | |
| 2 | 98910_at | Cluster Incl AI846118 UI-M-AK1-aew-e-07-0-UI.s1 Mus musculus cDNA, 3 end/clone = UI-M-AK1-aew-e-07-0-UI/ | P | 883.8 | P | 969.3 | P | 1374.4 | P |
| | | clone_end = 3/gb = AI846118/gi = 5489989/ug = Mm.28594/len = 234/STRA = for | | | | | | | |
| 2 | 98916_at | Cluster Incl AI854289 UI-M-BH0-ajs-e-06-0-UI s1 Mus musculus cDNA, 3 end/clone = UI-M-BH0-ajs-e-06-0-UI/ | P | 214.6 | P | 234.5 | A | 240.6 | P |
| | | clone_end = 3/gb-AI854289/gi = 5498195/ug = Mm 28618/len = 490/STRA = for | | | | | | | |
| 2 | 96848_at | Cluster Incl AW120756 UI-M-BH2 3-any-d-12-0-UI s1 Mus musculus cDNA, 3 end/clone = UI-M-BH2 3-any-d-12-0-UI/ | P | 83.5 | P | 99.7 | P | 148.9 | P |
| | | clone_end = 3/gb = AW120756/gi = 6096089/ug = Mm 30030/len = 407/STRA = for | | | | | | | |
| 2 | 97489_at | Cluster Incl AI846739 UI-M-AO1-ael-b-05-0-UI s1 Mus musculus cDNA, 3 end/clone = UI-M-AO1-ael-b-05-0-UI/ | P | 303.2 | P | 331 | P | 534.9 | P |
| | | clone_end = 3/gb = AI846739/gi = 5490645/ug = Mm 30969/len = 279/STRA = for | | | | | | | |
| 2 | 98561_at | Cluster Incl AJ242874 Mus musculus mRNA for slow skeletal muscle troponin 1 (Tnnl1 gene)/cds = (143,706)/gb = AJ242674/ | P | 364.1 | P | 166.6 | A | 756.4 | P |
| | | gi = 5457404/ug = Mm 36900/len = 1054/STRA = for | | | | | | | |
| 2 | 100086_at | Cluster Incl D00622 Low density lipoprotein receptor related protein, associated protein 1/cds = (0,1081)/gb = D00622/ | P | 154.1 | P | 265.1 | P | 332.9 | P |
| | | gi = 220433/ug = Mm 4560/len = 2902/STRA = for | | | | | | | |
| 2 | 101583_at | Cluster Incl M64292B-cell translocation gene 2, anti-proliferative/cds = (86,542)/gb = M64292/gi = 202068/ug = Mm 903/ | P | 845.7 | P | 1346 | P | 1327.7 | P |
| | | len = 2583/STRA = for | | | | | | | |
| 2 | 103423_at | Cluster Incl AI846517 UI-M-AN1-aff-e-02-0-UI s1 Mus musculus cDNA, 3 end/clone = UI-M-AN1-aff-e-02-0-UI/ | P | 313.5 | P | 281.5 | P | 409.4 | A |
| | | clone_end = 3/gb = AI846517/gi-5490423/ug = Mm 18942/len = 349/STRA = for | | | | | | | |
| 2 | 103622_at | Clusler Incl AW050255.UI-M-BH1-ans-b-11-0-UI s1 Mus musculus cDNA, 3 end/clone = UI-M-BH1-ans-b-11-0-UI/ | P | 406.7 | P | 346 | P | 450.9 | P |
| | | clone_end = 3/gb = AW050255/gi = 5910784/ug = Mm 20260/len = 397/STRA = for | | | | | | | |
| 2 | 103906_f_at | Cluster Incl AI840868 UI-M-AM0-adm-a-01-0-UI s1 Mus musculus cDNA. 3 end/clone = UI-M-AM0-adm-a-01-0-UI/ | P | 154 | P | 157.5 | P | 174.2 | P |
| | | clone_end = 3/gb = AI840868/gi = 5475081/ug = Mm 21401/len = 346/STRA = for | | | | | | | |
| 2 | 104701_at | Cluster Incl Y07836 M musculus mRNA for basic-helix-loop-helix protein/cds = (0,1214)/gb = Y07836/gi = 1707584/ | P | 214.2 | P | 195.6 | P | 230 | P |
| | | ug = Mm 2436/len = 1508/STRA = for | | | | | | | |
| 2 | 94978_at | Cluster Incl AIS89775 UI-M-AN0-acq-f-06-0-UI s1 Mus musculus cDNA, 3 end/clone = UI-M-AN0-acq-f-06-0-UI/ | P | 658.2 | P | 597.3 | P | 585 | P |
| | | clone_end = 3/gb = AI839775/gi = 5474028/ug = Mm 27269/len = 273/STRA = for | | | | | | | |
| 2 | 96227_at | Cluster Incl X70533 Corticosteroid binding globulin/cds = (54,1247)/gb = X70533/gi = 298114/ug = Mm 2874/len = 1462/ | P | 385.4 | P | 365.3 | M | 506.3 | P |
| | | STRA = for | | | | | | | |
| 2 | 96801_at | Cluster Incl AJ010108.Mus musculus mRNA for cytosolic adenylate Kinase/cds = (16,600)/gb = AJ010108/gi = 5725309/ | P | 164.1 | P | 92.7 | P | 222.3 | P |
| | | ug = Mm 29189/len = 1958/STRA = for | | | | | | | |
| 2 | 97960_at | Cluster Incl AW125800: UI-M-BH2.2-aql-h-05-0-UI.s1 Mus musculus cDNA, 3 end/clone = UI-M-BH2.2-aql-h-05-0-UI/ | P | 143.5 | P | 124.9 | A | 311 | P |
| | | clone_end = 3/gb = AW125800/gi = 6101330/ug = Mm.30602/len = 326/STRA = for | | | | | | | |
| 2 | 100952_at | Cluster Incl U47323: Stromal interaction molecule 1/cds = (193,2250)/gb = U47323/gi = 1493832/ug = Mm.645/len = 3192/ | A | 349.3 | P | 426.4 | P | 609.1 | P |
| | | STRA = for | | | | | | | |
| 2 | 101408_at | Cluster Incl AF010499: Guanldinoacetate methyltransferase/cds = (110,820)/gb = AF010499/gi = 2555082/ug = Mm 7329/ | P | 119.5 | M | 369.9 | P | 217.1 | P |
| | | len = 939/STRA = for | | | | | | | |
| 2 | 103731_at | Cluster Incl AI837319 UI-M-AK0-adf-c-02-0-UI.s1 Mus musculus cDNA, 3 end/clone = UI-M-AK0-adf-c-02-0-UI/ | P | 263.1 | P | 223.7 | P | 302.3 | A |
| | | clone end = 3/gb = AI837319/gi = 5471532/ug = Mm.24309/len = 418/STRA = rev | | | | | | | |
| 2 | 104156_r_at | Cluster Incl U19118 Mus musculus transcription factor LRG-21 mRNA, complete cds/cds = (167,712)/gb = U19118/ | P | 207.1 | P | 243.7 | A | 356.4 | P |
| | | gi = 633751/ug = Mm 2706/len = 1897/STRA = for | | | | | | | |
| 2 | 104262_at | Cluster Incl M60057: Parathyroid hormone-like peptide/cds = (25,552)/gb = M60057/gi = 200565/ug = Mm 28440/len = 1022/ | P | 304.4 | P | 555.3 | P | 489.8 | P |
| | | STRA = rev | | | | | | | |
| 2 | 92202_g_at | Cluster Incl AI553024: vf37g06 y1 Mus musculus cDNA, 5 end/clone = IMAGE-846010/clone_end = 5/gb = AI553024/ | P | 139.8 | P | 89.3 | P | 622.9 | P |
| | | gi = 4485387/ug = Mm 34106/len = 367/STRA = for | | | | | | | |
| 2 | 92235_g_at | Cluster Incl X66223: M musculus mRNA for retinoid X receptor-alpha (mRXR-alpha)/cds = (177,1580)/gb = X66223/ | P | 373.7 | P | 446.2 | P | 563.4 | P |
| | | gi = 54021/ug = Mm.3470/len = 2088/STRA = for | | | | | | | |
| 2 | 92382_at | Cluster Incl U49739 Myosin VI/cds = (159,3956)/gb = U49739/gi = 1322279/ug = Mm 4040/len = 4602/STRA = for | A | −30.5 | P | 3.4 | P | 26.1 | P |
| 2 | 92956_at | Cluster Incl X74760 Notch gene homolog 3, (Drosophila)/cds = (59,7015)/gb = X74760/gi = 483580/ug = Mm 4945/len = 7943/ | P | 380.8 | P | 435.4 | P | 429.9 | P |
| | | STRA = for | | | | | | | |
| 2 | 93921_at | Cluster Incl AI573601 ud46a12 y1 Mus musculus cDNA, 5 end/clone = IMAGE-1448926/clone_end = 5/gb = AI573601/ | P | 376.7 | P | 488.6 | P | 296.4 | A |
| | | gi = 4536975/ug = Mm.89855/len = 535/STRA = for | | | | | | | |
| 2 | 93958_at | Cluster Incl AA086863 mm86g02 r1 Mus musculus cDNA, 5 end/clone = IMAGE-535346/clone_end = 5/gb = AA086863/ | P | 204.7 | P | 189 | A | 265.2 | P |
| | | gi = 1630072/ug = Mm.95627/len = 494/STRA = for | | | | | | | |
| 2 | 94097_at | Cluster Incl AB025922 Mus musculus Gli1 mRNA, complete cds/cds = (171,3506)/gb = AB025922/gi = 6009643/ | P | 477.7 | P | 311 | A | 433.3 | P |
| | | ug = Mm 10081/len = 3686/STRA = for | | | | | | | |
| 2 | 95889_at | Cluster Incl AI854770 UI-M-BH0-aka-c-09-0-UI s1 Mus musculus cDNA, 3 end/clone = UI-M-BH0-aka-c-09-0-UI/ | P | 243 | P | 399 | P | 442.9 | M |
| | | clone_end = 3/gb = AI854770/gi = 5498676/ug = Mm 24046/len = 417/STRA = for | | | | | | | |
| 2 | 97762_f_at | Cluster Incl U12890 Killer cell lectin-like receptor, subfamily A, member 7/cds = (21,824)/gb = U12890/gi = 602409/ | M | 139.1 | P | 217.4 | P | 219.6 | P |
| | | ug = Mm 4131/len = 921/STRA = for | | | | | | | |
| 2 | 98277_at | Cluster Incl AB017615: Mus musculus mRNA for Eos protein, complete cds/cds = (752,2353)/gb = AB017615/gi = 4062982/ | P | 514.7 | P | 459.4 | M | 665.5 | P |
| | | ug = Mm 42034/len = 2688/STRA = for | | | | | | | |
| 2 | 100289_at | Cluster Incl U92885: Mus musculus LERK-3 (Epl3) gene, partial cds/cds = (0,564)/gb = U92885/gi = 2843098/ug = Mm 8547/ | P | 579.7 | P | 768.6 | P | 948.8 | A |
| | | len = 859/STRA = for | | | | | | | |
| 2 | 97652_at | Cluster Incl L07379 Growth hormone releasing hormone receptor/cds = UNKNOWN/gb = L07379/gi = 193510/ug = Mm 89928/ | P | 309.4 | P | 266.3 | P | 508.7 | P |
| | | len = 1320/STRA = for | | | | | | | |
| 2 | 101712_at | Cluster Incl AJ009823: Mus musculus mRNA for P2X7 receptor subunit/cds = (11,1798)/gb = AJ009323/gi = 4007637/ | P | 401.7 | P | 456.4 | P | 408.9 | P |
| | | ug = Mm.42026/len = 1810/STRA = for | | | | | | | |
| 2 | 102091_f_at | Cluster Incl AA407599.EST00874 Mus musculus cDNA, 3 end/clone = C0003E09/clone_end = 3/gb = AA407599/ | P | 73.9 | P | 89.7 | P | 121.2 | P |
| | | gi = 2065789/ug = Mm 42282/len = 285/STRA = rev | | | | | | | |
| 2 | 93799_at | Cluster Incl AI845933 UI-M-AK1-aex-f-02-0-UI.s1 Mus musculus cDNA, 3 end/clone = UI-M-AK1-aex-f-02-0-UI/ | P | 683.2 | P | 881.2 | P | 723.1 | A |
| | | clone_end = 3/gb = AI845933/gi = 5489839/ug = Mm 21407/len = 325/STRA = for | | | | | | | |
| 2 | 94208_at | Cluster Incl AW045202 UI-M-BH1-alh-c-08-0-UI.s1 Mus musculus cDNA, 3 end/clone = UI-M-BH1-alh-c-08-0-UI/ | P | 207.6 | P | 144.2 | P | 152.8 | A |
| | | clone_end = 3/gb = AW045202/gi = 5905731/ug = Mm.2220/len = 430/STRA = for | | | | | | | |
| 2 | 94274_at | Cluster Incl AI837415 UI-M-AP0-abm-f-06-0-UI s1 Mus musculus cDNA, 3 end/clone = UI-M-AP0-abm-f-06-0-UI/ | P | 134.4 | A | 492 | P | 565.5 | P |
| | | clone_end = 3/gb = AI837415/gi = 5471628/ug = Mm 22491/len = 360/STRA = for | | | | | | | |
| 2 | 102362_l_at | Cluster Incl U20735: Mus musculus transcription factor junB (junB) gene, 5 region and complete cds/cds = (294,1328)/ | P | 458.2 | P | 412.4 | A | 620.8 | P |
| | | gb = U20735/gi = 965003/ug = Mm 1167/len = 1786/STRA = for | | | | | | | |
| 2 | 102371_at | Cluster Incl X16995 Hormone receptor/cds = (2,1807)/gb = X16995/gi = 53312/ug = Mm.119/len = 2351/STRA = for | P | 84.8 | P | 35.9 | M | 311.5 | P |
| 2 | 104755_at | Cluster Incl AJ242778: Mus musculus mRNA for ABINI, (A20-binding inhibitor of NF-kappa B activation (large)/ | P | 331.6 | P | 407.2 | P | 438.9 | A |
| | | cds = (116,2059)/gb = AJ242778/gi = 4995752/ug = Mm 24782/len = 2812/STRA = rev | | | | | | | |
| 2 | 94336_at | Cluster Incl AI838318 UI-M-AO0-aby-f-01-0-UI s1 Mus musculus cDNA, 3 end/clone = UI-M-AO0-aby-f-01-0-UI/ | P | 426.8 | P | 387.9 | P | 413.9 | A |
| | | clone_end = 3/gb = AI838318/gi = 5472531/ug = Mm 2498/len = 186/STRA = rev | | | | | | | |
| 2 | 99991_at | Cluster Incl U31993.lnterleukin 17 receptor/cds = (120,2714)/gb = U31993/gi = 1161342/ug = Mm 4481/len = 3288/STRA = for | P | 184.8 | P | 147 | A | 174.6 | P |
| 2 | 103204_r_at | Cluster Incl AW259499 up28d02 x1 Mus musculus cDNA, 3 end/clone = IMAGE-2655651/clone_end = 3/gb = AW259499/ | P | 162.1 | P | 139.9 | P | 59.9 | A |
| | | gi = 6632417/ug = Mm.1911/len = 454/STRA = for | | | | | | | |
| 2 | 103551_at | Cluster Incl AW124208 UI-M-BH2 1-aph-c-03-0-UI.s1 Mus musculus cDNA, 3 end/clone = UI-M-BH2.1-aph-c-03-0-UI/ | P | 160.3 | P | 64.2 | P | 122.6 | P |
| | | clone_end = 3/gb = AW124208/gi = 6099738/ug = Mm 23813/len = 384/STRA = for | | | | | | | |
| 2 | 92500_at | Cluster Incl AB025412 Mus musculus mRNA for Ten-m3, complete cds/cds = (49,8196)/gb = AB025412/gi = 4760779/ | A | 26.7 | A | 14.4 | P | −2.1 | P |
| | | ug = Mm 42191/len = 8964/STRA = for | | | | | | | |
| 2 | 92923_g_at | Cluster Incl U17291: Mus musculus transcription factor AP-2 (AP-2) gene, alternative/cds = (36,602)/gb = U17291/ | A | 105.8 | P | 174.6 | P | 411.3 | P |
| | | gi = 755199/ug = Mm 4845/len = 603/STRA = for | | | | | | | |
| 2 | 93718_at | Cluster Incl Z22821 Mus musculus mRNA for Rab23 protein/cds = (20,733)/gb = Z22821/gi = 438161/ug = Mm 86744/ | P | 49.4 | P | 55.3 | A | 91.1 | P |
| | | len = 740/STRA = for | | | | | | | |
| 2 | 93724_at | Cluster Incl AI596034 uk22a01.x1 Mus musculus cDNA, 3 end/clone = IMAGE-1969704/clone_end = 3/gb = AI596034/ | A | 154.4 | P | 152.6 | P | 161.8 | P |
| | | gi = 4605082/ug = Mm.86922/len = 516/STRA = rev | | | | | | | |
| 2 | 94133_r _at | Cluster Incl U94423: Mus musculus MEF2A mRNA, partial 3 UTR sequence/cds = UNKNOWN/gb = U94423/gi = 2088559/ | P | 43.3 | P | 59.4 | P | 52.6 | A |
| | | ug = Mm 11087/len = 1016/STRA = rev | | | | | | | |
| 2 | 95360_at | Cluster Incl AA259683: va36d09 r1 Mus musculus cDNA, 5 end/clone = IMAGE-733457/clone_end = 5/gb = AA259683/ | P | 2.5 | P | −1.6 | P | 10.5 | P |
| | | gi = 1896186/ug = Mm.21902/len = 349/STRA = for | | | | | | | |
| 2 | 97171_f_at | Cluster Incl AI060627 Ub42b11.r1 Mus musculus cDNA, 5 end/clone = IMAGE-1380381/clone_end = 5/gb = AI060627/ | P | 211.7 | P | 189.5 | A | 534.3 | P |
| | | gi = 3336050/ug = Mm 35621/len = 335/STRA = for | | | | | | | |
| 2 | 98291_at | Cluster Incl AF092050: Mus musculus beta-1,3-N-acetylglucosaminyltransferase mRNA, complete cds/cds = (4,981)/ | M | 212.2 | P | 209 | A | 357.5 | P |
| | | gb = AF092050/gi = 4191391/ug = Mm.42092/len = 1536/STRA = for | | | | | | | |
| 2 | 101769_at | Cluster Incl AB008178: Mus musculus CNR3 mRNA for Cadherin-related neural recepter 3, partial cds/cds = (0,2730)/ | P | 798.3 | P | 1006.1 | M | 1099.4 | M |
| | | gb = AB008178/gi = 3253078/ug = Mm.57101/len = 2731/STRA = for | | | | | | | |
| 2 | 101603_g_at | Cluster Incl C79108: C79108 Mus musculus cDNA, 3 end/clone = J0060E04/clone_end = 3/gb = C79108/gi = 2519438/ | M | 193.9 | P | 189.2 | P | 258.6 | P |
| | | ug = Mm 5769/len = 579/STRA = rev | | | | | | | |
| 2 | 92768_s_at | Cluster Incl M15268.Aminolevulinic acid synthase 2, erythroid/cds = (0,1760)/gb = M15268/gi = 191857/ug = Mm 1217/ | A | 181.1 | P | 190.3 | P | 1782.7 | P |
| | | len = 1896/STRA = for | | | | | | | |
| 2 | 93073_at | Cluster Incl U36576 Nuclear factor of activated T-cells, cytoplasmic 2/cds = (70,2853)/gb = U36576/gi = 1353400 | P | 949.3 | P | 351.7 | A | 1606.6 | A |
| | | ug = Mm 1574/len = 3518/STRA = rev | | | | | | | |
| 2 | 93583_s_at | Cluster Incl V00817: Mouse germ line gene fragment for mu-immunoglobulin C-terminus (secreted form)/cds = (0,83)/ | P | 332 | P | 428.2 | M | 354.3 | A |
| | | gb = V00817/gi = 52345/ug = Mm 2066/len = 210/STRA = for | | | | | | | |
| 2 | 94046_at | Cluster Incl U05247 C-src tyrosine kinase/cds = (0,1352)/gb = U05247/gi = 452471/ug = Mm.21974/len = 1779/STRA = for | P | 459.6 | P | 578.2 | P | 551.5 | P |
| 2 | 94856_r_at | Cluster Incl AI842362 UI-M-AM1-agc-a-03-0-UI s1 Mus musculus cDNA, 3 end/clone = UI-M-AM1-agc-a-03-0-UI/ | A | 57.3 | A | 37.5 | P | 105.8 | P |
| | | clone_end = 3/gb = AI842362/gi = 5476575/ug = Mm 23566/len = 298/STRA = for | | | | | | | |
| 2 | 96026_at | Cluster Incl AA987153: uc80h10.x1 Mus musculus cDNA, 3 end/clone = IMAGE-1432003/clone_end = 3/gb = AA987153/ | P | 101.7 | P | 80.6 | A | 127.2 | A |
| | | gi = 3167916/ug = Mm.2573/len = 824/STRA = for | | | | | | | |
| 2 | 97906_at | Cluster Incl Z19581.Seven in absentia 2/cds = (289,1266)/gb = Z19581/gi = 297036/ug = Mm 2847/len = 2198/STRA = for | P | 343.6 | P | 385.6 | A | 521.5 | A |
| 2 | 99186_at | Cluster Incl X75483 Cyclin A2/cds = (121,1389)/gb = X75483/gi = 414061/ug = Mm.4189/len = 1662/STRA = for | A | 394 | P | 485 | A | 467 | P |
| 2 | 101063_at | Cluster Incl M29793: M musculus slow/cardiac troponin C (cTnC) mRNA, complete cds/cds = (43,528)/gb = M29793/ | P | 219.3 | P | 89.6 | A | 820.9 | P |
| | | gi = 192839/ug = Mm 712/len = 703/STRA = for | | | | | | | |
| 2 | 101505_at | Cluster Incl AW121234 UI-M-BH2 3-aok-f-04-0-UI s1 Mus musculus cDNA, 3 end/clone = UI-M-BH2 3-aok-f-04-0-UI/ | P | 234.4 | P | 136.7 | A | 178.4 | P |
| | | clone_end = 3/gb = AW121234/gi = 6096567/ug = Mm 8195/len = 426/STRA = for | | | | | | | |
| 2 | 94947_g_at | Cluster Incl U43187: Mus musculus MEK kinase 3, mRNA, partial cds/cds = (331,2211)/gb = U43187/gi = 1223901/ | P | 85.4 | P | 68 | A | 127.9 | P |
| | | ug = Mm 27041/len = 3332/STRA = for | | | | | | | |
| 2 | 97358_at | Cluster Incl AI851356 UI-M-BH0-akh-d-11-0-UI.s1 Mus musculus cDNA, 3 end/clone = UI-M-BH0-akh-d-11-0-UI/ | P | 357.7 | P | 515.6 | P | 366.3 | P |
| | | clone_end = 3/gb = AI851356/gi = 5495262/ug = Mm.29638/len = 401/STRA = for | | | | | | | |
| 2 | 97942_g_at | Cluster Incl Y12582 M musculus mRNA for calpain-like protease/cds = (129,2054)/gb = Y12582/gi = 2239139/ug = .Mm 30290/ | P | 378.7 | P | 202.1 | A | 242.5 | P |
| | | len = 2069/STRA = for | | | | | | | |
| 2 | 100432_f_at | Cluster Incl U57820 MyoD family inhibitor/cds = (30,770)/gb = U57820/gi = 1545983/ug = Mm 4763/len = 1360/STRA = for | P | 742.3 | A | 1154.9 | A | 996 | P |
| 2 | 93669_f_at | Cluster lncl AW107922 um24d07 x1 Mus musculus cDNA, 3 end/clone = IMAGE-2225485/clone_end = 3/gb = AW107922/ | P | 2.1 | A | 77.2 | P | 37.6 | M |
| | | gi = 6078722/ug = Mm 80595/len = 576/STRA = rev | | | | | | | |
| 2 | 94770_at | Cluster Incl AI553596 vw47f05.x1 Mus musculus cDNA, 3 end/clone = IMAGE-1246977/clone_end = 3/gb = AI553596/ | P | 208.9 | P | 243.6 | P | 294 | A |
| | | gi = 4485959/ug = Mm 1648/len = 341/STRA = for | | | | | | | |
| 2 | 95340_at | Cluster Incl M93310 Mouse metailothionein-III gene, complete cds/cds = (56,262)/gb = M93310/gi = 199133/ug = Mm 2064/ | P | 821.7 | P | 922.2 | P | 964.7 | P |
| | | len = 401/STRA = for | | | | | | | |
| 2 | 97769_at | Cluster Incl D17433 Prostaglandin F receptor/cds = (223,1323)/gb = D17433/gi = 457656/ug = Mm.4170/len = 2170/ | P | 223.4 | P | 141 | A | 154.3 | A |
| | | STRA = rev | | | | | | | |
| 2 | 100736_at | Cluster Incl L77900 Mus musculus Barx2 mRNA, complete cds/cds = (67,753)/gb = L77900/gi = 2055363/ug = Mm 3508/ | A | 66.7 | A | 28.1 | P | 127.8 | P |
| | | len = 1274/STRA = for | | | | | | | |
| 2 | 92619_at | Cluster Incl U40825 Mus musculus WW-domain binding protein 1 mRNA, complete cds/cds = (152,1069)/gb = U40825/ | M | 1024.5 | P | 1320.4 | M | 1459.3 | A |
| | | gi = 1777576/ug = Mm 1109/len = 1292/STRA = for | | | | | | | |
| 2 | 92820_at | Cluster Incl AI846522 UI-M-AN1-aff-e-09-0-UI.s1 Mus musculus cDNA, 3 end/clone = UI-M-AN1-aff-e-09-0-UI/ | P | 403.2 | P | 338.8 | A | 573.2 | P |
| | | clone_end = 3/gb = AI846522/gi = 5490428/ug = Mm 12914/len = 370/STRA = rev | | | | | | | |
| 2 | 93507_at | Cluster Incl X62622 Tissue inhibitor of metalloproteinase 2/cds = (226,888)/gb = X62622/gi = 54801/ug = Mm 19191/ | A | 205.5 | P | 162.9 | A | 229.1 | A |
| | | len = 1714/STRA = for | | | | | | | |
| 2 | 94815_at | Cluster Incl X13586 Murine mRNA for 2,3-bisphosphoglycerate mutase (BPGM; EC 5.4,2.4)/cds = (20,799)/gb = X13586/ | A | 161.6 | P | 100.2 | P | 394.6 | P |
| | | gi = 50183/ug = Mm 22706/len = 825/STRA = for | | | | | | | |
| 2 | 94906_at | Cluster Incl M22679 Mouse alcohol dehydrogenase class I gene (ADH-A-2)/cds = (0,1127)/gb = M22679/gi = 191715/ | A | 410.9 | P | 161.7 | P | 647 | P |
| | | ug = Mm.2409/len = 1128/STRA = for | | | | | | | |
| 2 | 101046_at | Cluster Incl X56397: Mouse mRNA for vimentin/cds = (84,1484)/gb = X56397/gi = 55407/ug = Mm 7/len = 1748/STRA = for | P | 252.6 | M | 397.1 | P | 339.8 | A |
| 2 | 101531_at | Cluster Incl AI527354.uj51b11.x1 Mus musculus cDNA, 3 end/clone = IMAGE-1923453/clone_end = 3/gb = AI527354/ | P | 333.4 | P | 228.2 | A | 1227.9 | A |
| | | gi = 4441489/ug = Mm 87581/len = 534/STRA = rev | | | | | | | |
| 2 | 102429_at | Cluster Incl AB005451: Mus musculus mRNA for RST, complete cds/cds = (99,1760)/gb = AB005451/gi = 2696708/ | P | 486.5 | P | 400.2 | M | 577.3 | A |
| | | ug = Mm.12846/len = 1779/STRA = for | | | | | | | |
| 2 | 104047_at | Cluster Incl AW122136: UI-M-BH2.2-aor-d-08-0-UI s1 Mus musculus cDNA, 3 end/clone = UI-M-BH2 2-aor-d-08-0-UI/ | P | 201 | P | 114.1 | M | 367.5 | P |
| | | clone end = 3/gb = AW122136/gi = 6097563/ug = Mm 21495/len = 276/STRA = for | | | | | | | |
| 2 | 99021_at | Cluster Incl U03873 Paired masoderm homeobox 1/cds = (1021,1674)/gb = U03873/gi = 460124/ug = Mm 3869/len = 3359/ | P | 78.1 | P | 78.9 | A | 108.6 | A |
| | | STRA = for | | | | | | | |
| 2 | 99055_at | Cluster Incl X94000: M. musculus Gal beta-1,3-GaINAc-specific GaINAc alpha-2,6-aiatyltransferase gene (1804 bp)/ | A | 835.6 | P | 539.1 | P | 680.1 | P |
| | | cds = (67,153)/gb-X94000/gi = 1526610/ug = Mm.3947/len = 154/STRA = for | | | | | | | |
| 2 | 100449_g_at | Cluster Incl Z31664 M musculus (NIH Swiss) mRNA for ALK-1/cds = (240,1748)/gb = Z31684/gi = 840814/ug = Mm 4839/ | P | 312.4 | P | 255.1 | A | 258.2 | P |
| | | len = 1909/STRA = for | | | | | | | |
| 2 | 103211_at | Cluster Incl AI852574: UI-M-BH0-aiu-a-02-0-UI s1 Mus musculus cDNA, 3 end/clone = UI-M-BH0-alu-a-02-0-UI/ | A | 836.7 | P | 700.1 | P | 1011.4 | P |
| | | clone_end = 3/gb = AI852574/gi = 5496480/ug = Mm 1960/len = 235/STRA = rev | | | | | | | |
| 2 | 103789_at | Cluster Incl AI854614: UI-M-BH0-ake-d-06-0-UI s1 Mus musculus cDNA, 3 end/clone = UI-M-BH0-ake-d-06-0-UI/ | P | 79.1 | A | 291.2 | P | 272.8 | A |
| | | clone_end = 3/gb = AI854614/gi = 5498520/ug = Mm 24948/len = 446/STRA = for | | | | | | | |
| 2 | 92532_at | Cluster Incl D49730: Mouse mRNA for V1a agrinine vasopression receptor, complete cds/cds = (312,1583)/gb = D49730/ | P | 238.1 | P | 291.1 | A | 185.1 | P |
| | | gi = 1722700/ug = Mm.4351/len = 2684/STRA = rev | | | | | | | |
| 2 | 93870_at | Cluster Incl M64429 Braf transforming gene/cds = (37,2016)/gb = M64429/gi = 553877/ug = Mm 8807/len = 2050/STRA = for | P | 89.9 | P | 109.1 | P | 114.7 | M |
| 2 | 94142_at | Cluster Incl M13926 Colony stimulating factor, granulocyte/cds = (87,693)/gb = M13926/gi = 193451/ug = Mm 1238/len = 1363/ | P | 146.6 | P | 455.4 | A | 972.4 | P |
| | | STRA = for | | | | | | | |
| 2 | 94784_at | Cluster Incl AI593484 vu42d01 x1 Mus musculus cDNA, 3 end/clone = IMAGE-1194049/clone_end = 3/gb = AI593484/ | A | 199.1 | P | 259.9 | A | 202.9 | A |
| | | gi = 4602532/ug = Mm.1689/len = 477/STRA = for | | | | | | | |
| 2 | 95881_f_at | Cluster Incl AI447783 mq84c02.x1 Mus musculus cDNA, 3 end/clone = IMAGE-585410/clone_end = 3/gb = AI447783/ | P | 295.3 | P | 332.4 | A | 353.3 | A |
| | | gi = 4306405/ug = Mm.23936/len = 347/STRA = rev | | | | | | | |
| 2 | 96574_at | Cluster Incl M30136 Interleukin 9/cds = (0,434)/gb = M30136/gi = 200190/ug = Mm 3006/len = 435/STRA = for | P | 237.1 | A | 189 | A | 300.4 | P |
| 2 | 97147_at | Cluster Incl C80892: C80892 Mus musculus cDNA, 3 end/clone = J0090C03/clone_end = 3/gb = C80892/gi = 2521222/ | M | 7.8 | A | 52.3 | P | 27.8 | A |
| | | ug = Mm 34947/len = 585/STRA = rev | | | | | | | |
| 2 | 100291_at | Cluster Incl X57111.Casitas B-lineage lymphoma/cds = (96,2786)/gb = X57111/gi = 50314/ug = Mm.8585/len = 2808/ | M | 38.9 | P | 18.3 | P | 76.8 | P |
| | | STRA = for | | | | | | | |
| 2 | 94270_at | Cluster Incl M22832 Mus musculus cytokeratin (endoB) gene, complete cds/cds = (58,1329)/gb = M22832/gi = 340757/ | P | 33.9 | P | 93.4 | A | 146.4 | A |
| | | ug = Mm 22479/len = 1373/STRA = for | | | | | | | |
| 2 | 94308_at | Cluster Incl X70853.BM-90/fibulin extracellular matrix glyroprotein/cds = (0,353)/gb = X70853/gi = 396816/ug = Mm 22626/ | A | 388.8 | P | 565.9 | M | 481.1 | P |
| | | len = 448/STRA = for | | | | | | | |
| 2 | 100130_at | Cluster Incl X12761: Jun oncogene/cds = (354,1358)/gb = X12761/gi = 52758/ug = Mm 482/len = 2565/STRA = for | P | 174.5 | P | 159 | P | 232 | M |
| 2 | 102071_at | Cluster Incl AIB51565: UI-M-BHO-aki-f-01-0-UI s1 Mus musculus cDNA, 3 end/clone = UI-M-BH0-aki-f-01-0-UI/clone_end = 3/ | P | 464.2 | P | 459.5 | A | 623.7 | M |
| | | gb = AI851565/gi = 5495471/ug = Mm 10641/len = 341/STRA = for | | | | | | | |
| 2 | 103088_at | Cluster Incl X94310: M musculus mRNA for L1-like protein/cds = (295,3924)/gb = X94310/gi = 1532034/ug = Mm 1736/ | M | 72.7 | P | 91.5 | A | 104.6 | P |
| | | len = 4437/STRA = for | | | | | | | |
| 2 | 103420_at | Cluster Incl U79753 Emerin/cds = (106,885)/gb = U79753/gi = 1916802/ug = Mm 18892/len = 1270/STRA = for | P | 754.3 | P | 880 | A | 922.3 | A |
| 2 | 104393_at | Cluster Incl AI843571 UI-M-AO1-aen-d-09-0-UI.s1 Mus musculus cDNA, 3 end/clone = UI-M-AO1-aen-d-09-0-UI/ | M | −79.9 | A | 4.3 | P | 26.3 | P |
| | | clone_end = 3/gb = AI843571/gl = 5477824/ug = Mm 2275/len = 290/STRA = for | | | | | | | |
| 2 | 95566_at | Cluster Incl AF050666: Glycosylphosphatidytinositol specific phospholipase D1/cds = (270,2783)/gb = AF050666/ | A | 302.2 | A | 281.2 | P | 334.1 | P |
| | | gi = 2984690/ug = Mm.2779/len = 3436/STHA = for | | | | | | | |
| 2 | 100015_at | Cluster Incl X67677 Yamaguchi sarcoma viral (v-yes) oncogene homolog/cds = (602,2227)/gb = X67677/gi = 50623/ | P | 347.5 | P | 193.3 | P | 335.1 | A |
| | | ug = Mm 4558/len = 4550/STRA = for | | | | | | | |
| 2 | 102202_s_at | Cluster Incl M36411 Mpv17 transgene, kidney disease mutant/cds = (48,578)/gb = M36411/gi = 199789/ug = Mm 999/ | P | 185.4 | P | 163.7 | P | 186.9 | P |
| | | len = 1411/STRA = rev | | | | | | | |
| 2 | 103214_at | Cluster Incl AW048883 UI-M-BH1-amf-f-01-0-UI s1 Mus musculus cDNA, 3 end/clone = UI-M-BH1-amf-f-01-0-UI/ | P | 26.1 | M | 79.9 | A | 165.7 | P |
| | | clone_end = 3/gb = AW048883/gi = 5909412/ug = Mm 1980/len = 489/STRA = for | | | | | | | |
| 2 | 104455_at | Cluster Incl J03057 Mouse brain Ca++/calmodulin-dependant protein kinase type IV (CAM kinase IV) mRNA, 3 end/ | P | 54.9 | A | 69.5 | P | 91.9 | A |
| | | cds = (0,667)/gb = J03057/gi = 192366/ug = Mm 2951/len = 773/STRA = for | | | | | | | |
| 2 | 93619_at | Cluster Incl AF022992 Period homolog (Drosophila)/cds = (225,4100)/gb = AF022992/gi = 2435508/ug = Mm 7373/len = 4701/ | P | 108.3 | P | 142.5 | A | 381.8 | P |
| | | STRA = for | | | | | | | |
| 2 | 94748_g_at | Cluster Incl M34397.Interieukin 3 receptor, beta chain 1/cds = (287,2977)/gb = M34397/gi = 191821/ug = Mm 14595/ | P | 209.6 | P | 176.5 | M | 279.5 | A |
| | | len = 3113/STRA = for | | | | | | | |
| 2 | 96592_at | Cluster Incl U50413 Phosphatidylinositol 3-kinase, regulatory subunit, polypeptide 1 (p85 alpha)/cds = (574,2748)/ | P | −4.6 | A | 110.7 | P | 105.5 | A |
| | | gb = U50413/gi = 1621039/ug = Mm.3058/len = 3454/STRA = rev | | | | | | | |
| 2 | 97169_f_at | Cluster Incl AF067058 Mus musculus clone L10 variable group of 2-cell-stage gene family mRNA, complete cds/ | P | 91.1 | P | 68.3 | A | 124.9 | A |
| | | cds = (166,675)/gb = AF067058/gi = 3171133/ug = Mm 35599/len = 874/STRA = for | | | | | | | |
| 2 | 98840_at | Cluster Incl AF012104 Mus musculus cytoplasmic tyrosine kinase (bmx) mRNA, complete cds/cds = (8,1963)/ | P | 28.4 | A | 78.3 | P | 97.3 | P |
| | | gb = AF012104/gi = 2293557/ug = Mm 504/len = 2369/STRA = for | | | | | | | |
| 2 | 100280_at | Cluster Incl X72805 M musculus gene for testicular hislone H1/cds = (0,626)/gb = X72805/gi = 1934959/ug = Mm 8048/ | A | 214.6 | M | 223.4 | M | 372.6 | A |
| | | len = 627/STRA = for | | | | | | | |
| 2 | 100742_at | Cluster Incl AA409749 EST01534 Mus musculus cDNA, 3 end/clone = C0011A07/clone_end = 3/gb = AA409749/ | P | −5.7 | A | 136 | P | 173.1 | A |
| | | gi = 2066449/ug = Mm.3628/len = 337/STRA = rev | | | | | | | |
| 2 | 101168_at | Cluster Incl Z31360.M. musculus (Balb/C) P/L01 mRNA/cds = (0,139)/gb = Z31360/gi = 467579/ug = Mm 5029/len = 421/ | M | 54.5 | P | 43.2 | A | 68.1 | P |
| | | STRA = for | | | | | | | |
| 2 | 101185_at | Cluster Incl X92498 Forkhead homolog 6, (Drosophila)/cds = (277,1290)/gb = X92498/gi = 1199829/ug = Mm.5111/len = 1761/ | A | 271.8 | A | 263.6 | M | 467.4 | P |
| | | STRA = for | | | | | | | |
| 2 | 99585_at | Cluster Incl AI894122: md85f04 x1 Mus musculus cDNA, 3 end/clone = IMAGE-375199/clone_end = 3/gb = AI894122/ | P | 390.1 | P | 537 | A | 622.5 | A |
| | | gi = 5600024/ug = Mm 4261/len = 425/STRA = for | | | | | | | |
| 2 | 102401 _at | Cluster Incl M21085 Interferon regulatory factor 1/cds = (205,1194)/gb = M21065/gi = 198458/ug = Mm.1246/len = 2072/ | P | 541.2 | P | 435.8 | A | 812.5 | A |
| | | STRA = for | | | | | | | |
| 2 | 103084_at | Cluster Incl D88791 Mouse mRNA for muscle LIM protein, complete cds/cds = (0,584)/gb = D88791/gi = 1695699/ | P | 122.6 | A | −63.1 | A | −49 | A |
| | | ug = Mm 17235/len = 585/STRA = for | | | | | | | |
| 2 | 103573_at | Cluster Incl D86176.Mouse mRNA for phosphatidylinositol 4-phosphate 5-kinase type I-alpha, complete cds/cds = (84,1703)/ | P | 74.5 | A | 46 | A | 106.8 | M |
| | | gb = D86176/gi = 1622707/ug = Mm 1945/len = 2378/STRA = for | | | | | | | |
| 2 | 103854_at | Cluster Incl AW049387 UI-M-BH1-ane-d-08-0-UI s1 Mus musculus cDNA, 3 end/clone = UI-M-BH1-ane-08-0-UI/ | P | 56.3 | M | 39.8 | A | 87.6 | P |
| | | clone_end = 3/gb = AW049387/gi = 5909916/ug = Mm.21071/len = 335/STRA = for | | | | | | | |
| 2 | 104342_i_at | Cluster Incl AI845798.UI-M-AQ1-aeb-h-07-0-UI s1 Mus musculus cDNA, 3 end/clone = UI-M-AQ1-aab-h-07-0-UI/ | P | −28.5 | P | −105 | A | −70.2 | P |
| | | clone_end = 3/gb = AI845798/gi = 5489704/ug = Mm.22526/len = 184/STRA = for | | | | | | | |
| 2 | 96160_at | Cluster Incl X67056: Glycine transporter 1/cds = (240,2141)/gb = X67058/gi = 51092/ug = Mm 2834/len = 3227/STRA = for | P | 469.5 | P | 429.1 | A | 432 | A |
| 2 | 96702_at | Cluster Incl AI787317: uj66c05 x1 Mus musculus cDNA, 3 end/clone = IMAGE-1924804/clone_end = 3/gb = AI787317/ | P | 74.8 | P | 26.2 | A | 100.8 | A |
| | | gi = 5885993/ug = Mm 20123/len = 470/STRA = for | | | | | | | |
| 2 | 92205_at | Cluster Incl AF090738: Mus musculus insulin receptor substrate-2 (Irs2) gene, partial cds/cds = (0,2206)/gb = AF090738/ | P | 173.7 | A | 315.6 | A | 283.1 | A |
| | | gi = 3661524/ug = Mm.34143/len = 2207/STRA = for | | | | | | | |
| 2 | 92254_at | Cluster Incl M55253: Myosin Vb/cds = (46.2217)/gb = M55253/gi = 193414/ug = Mm 3536/len = 2632/STRA = for | P | −26.2 | P | −98.1 | A | 25.8 | A |
| 2 | 98290_at | Cluster Incl AF027185 Mus musculus ELP mRNA, complete cds/cds = (35,430)/gb = AF027185/gi = 4103746/ug = Mm 42090/ | P | 334.3 | P | 397 | A | 440.2 | A |
| | | len = 1081/STRA = for | | | | | | | |
| 2 | 98770_at | Cluster Incl AF012708: Mus musculus centromere protein C (Cenp-c) gene, promoter region and partial cds/cds = (142,159)/ | P | 54.6 | P | −69.9 | A | 85.8 | A |
| | | gb = AF012708/gi = 2465200/ug = Mm 4649/len = 160/STRA = for | | | | | | | |
| 2 | 101225_at | Cluster Incl C77257 C77257: Mus musculus cDNA, 3 end/clone = J0028804/clone_end = 3/gb = C77257/gi = 2517587/ | P | 188.7 | P | 225.4 | P | 230.5 | A |
| | | ug = Mm 5593/len = 501/STRA = rev | | | | | | | |
| 3 | 92575_at | Cluster Incl U85489: Mus musculus Ah receptor-interacting protein (AIP) mRNA, complete cds/cds = (209,1201)/gb = U85489/ | P | 555.4 | P | 684.9 | P | 796.6 | P |
| | | gi = 2177173/ug = Mm 10433/len = 1367/STRA = for | | | | | | | |
| 3 | 92635_at | Cluster Incl M13444 Mouse alpha-tublin isotype M-alpha-4 mRNA, complete cds/cds = (29,1375)/gb = M13444/gi = 202214/ | P | 558.4 | P | 574.8 | P | 813.2 | P |
| | | ug = Mm 1155/len = 1442/STRA = for | | | | | | | |
| 3 | 92642_at | Cluster Incl M25944 Mouse carbonic anhydrase II (CAII) mRNA, 3 end/cds = (0,62)/gb = M25944/gi = 199078/ug = Mm 1186/ | P | 132.7 | P | 117.1 | P | 325.5 | P |
| | | len = 742/STRA = for | | | | | | | |
| 3 | 92778_i_at | Cluster Incl Z22552: M musculus membrane glycoprotein gene/cds = (0,223)/gb = Z22552/gi = 296166/ug = Mm. 1232 | P | 745.8 | P | 787.2 | P | 1049.5 | P |
| | | len = 224/STRA = for | | | | | | | |
| 3 | 92821_at | Cluster Incl AF079565: Mus musculus ubiquitin-specific protease UBP41 (Uop41) mRNA, complete cds/cds = (0,1061)/ | P | 214 | P | 290.5 | P | 520.1 | P |
| | | gb = AF079565/gi = 3386551/ug = Mm 12914/len = 1115/STRA = for | | | | | | | |
| 3 | 92836_at | Cluster Incl AA919594 vz22b07 r1 Mus musculus cDNA, 5 end/clone = IMAGE-1316437/clone_end = 5/gb = AA919594/ | P | 2318 | P | 3134.5 | P | 3846.8 | P |
| | | gi = 3066373/ug = Mm. 13097/len = 222/STRA = for | | | | | | | |
| 3 | 92852_at | Cluster Incl M18194 Mouse fibronectin (FN) mRNA/cds = (0,311)/gb = M18194/gi = 193329/ug = Mm 13846/len = 905/ | P | 1877.4 | P | 2025.3 | P | 2843.4 | P |
| | | STRA = for | | | | | | | |
| 3 | 92863_at | Cluster Incl U40826 Mus musculus WW-domain binding protein 2 mRNA, complete cds/cds = (39,824)/gb = U40826/ | P | 455.2 | P | 432.6 | P | 557.7 | P |
| | | gi = 1777578/ug = Mm 1405/len = 1802/STRA = for | | | | | | | |
| 3 | 92885_at | Cluster Incl L48989: Troponin T3, skeletal, fast/cds = (0,719)/gb = L48989/gi = 2340049/ug = Mm 14546/len = 720/STRA = for | P | 2449.3 | P | 3501.7 | P | 5922 | P |
| 3 | 93011_at | Cluster Incl AW123904 UI-M-BH2.1-app-g-10-0-UI s1 Mus musculus cDNA, 3 end/clone = UI-M-BH2.1-app-g-10-0-UI/ | P | 649.1 | P | 724 | P | 1010.4 | P |
| | | clone_end = 3/gb = AW123904/gi = 6099434/ug = Mm.14638/len = 433/STRA = for | | | | | | | |
| 3 | 93048_at | Cluster Incl AJ005253 Mus musculus mRNA for CipP protease/cds = (32,850)/gb = AJ005253/gi = 3559934/ug = Mm.15243/ | P | 316.2 | P | 376.8 | P | 478.7 | P |
| | | len = 990/STRA = for | | | | | | | |
| 3 | 93053_at | Cluster Incl AF068244 Mus musculus cardiac calsequesirin mRNA, complete cds/cds = (129,1376)/gb = AF068244/ | P | 317.6 | P | 316.7 | P | 705.9 | P |
| | | gi = 3219362/ug = Mm.15343/len = 2275/STRA = for | | | | | | | |
| 3 | 93063_at | Cluster Incl U82624 Mus musculus amyloid precursor protein (APP) gene, partial cds, exons 16 and 17/cds = (0,247)/ | P | 2689.3 | P | 3304.9 | P | 3500.4 | P |
| | | gb = U82624/gi = 1778801/ug = Mm 15571/len = 248/STRA = for | | | | | | | |
| 3 | 93084_at | Cluster Incl U27315 Adenine nucleotide translocator 1, skeletal muscle/cds = (86,982)/gb = U27315/gi = 902007/ | P | 2055.1 | P | 2753.3 | P | 3915.4 | P |
| | | ug = Mm.16228/len = 1273/STRA = for | | | | | | | |
| 3 | 93086_at | Cluster Incl M18237 Mouse lg kappa chain V-region mRNA, partial cds, clone 3H9/cds = (0,321)/gb = M18237/gi = 197007/ | P | 202.7 | P | 205 | P | 360.1 | P |
| | | ug = Mm 16253/len = 322/STRA = for | | | | | | | |
| 3 | 93284_at | Cluster Incl D78135 Mus musculus mRNA for CIRP, complete cds/cds = (82,600)/gb = D78135/gi = 1060918/ug = Mm 17898/ | P | 862.3 | P | 831.2 | P | 1619.8 | P |
| | | len = 1256/STRA = for | | | | | | | |
| 3 | 93330_at | Cluster Incl L02914 Aquaporin 1/cds = (59,868)/gb = L02914/gi = 193642/ug = Mm 18625/len = 2627/STRA = for | P | 423.9 | P | 359.3 | P | 632.7 | P |
| 3 | 93490_at | Cluster Incl AI841771 UI-M-AN0-acl-c-12-0-UI s1 Mus musculus cDNA, 3 end/clone = UI-M-AN0-acl-c-12-0-UI/ | P | 502.2 | P | 479 | P | 654.2 | P |
| | | clone_end = 3/gb = AI841771/gi = 5475984/ug = Mm 19055/len = 479/STRA = for | | | | | | | |
| 3 | 93500_at | Cluster Incl M63245 Mus musculus amino levulinate synthase (ALAS-H) mRNA, 3 end/cds = (0,1369)/gb = M63245/ | P | 596.5 | P | 406.5 | P | 1124.4 | P |
| | | gi = 1220401/ug = Mm 19143/len = 1448/STRA = for | | | | | | | |
| 3 | 93511_at | Cluster Incl L38971 Integral membrane protein 2/cds = (147,938)/gb = L38971/gi = 624777/ug = Mm 193/len = 1635/ | P | 2601.4 | P | 3192.7 | P | 3774.7 | P |
| | | STRA = for | | | | | | | |
| 3 | 93540_at | Cluster Incl L13290 Mus musculus ADP-ribosylarginine hydrolase mRNA, complete cds/cds = (0,1088)/gb = L13290/ | P | 578.1 | P | 582.4 | P | 712.8 | P |
| | | gi = 402489/ug = Mm 20047/len = 1089/STRA = for | | | | | | | |
| 3 | 93732_f_at | Cluster Incl AF104358.Mus musculus synectin mRNA, complete cds/cds = (72,1073)/gb = AF104358/gi = 4219017/ | P | 729.5 | P | 914.5 | P | 972 | P |
| | | ug = Mm 20945/len = 1600/STRA = for | | | | | | | |
| 3 | 93736_at | Cluster Incl AF090686: Mus musculus transcobalmin II (Tcn2) mRNA, complete cds/cds = (127,1419)/gb = AF090686/ | P | 326.2 | P | 505 | P | 545.3 | P |
| | | gi = 3659876/ug = Mm.20948/len = 1878/STRA = for | | | | | | | |
| 3 | 93767_i_at | Cluster Incl AW124052 UI-M-BH2.1-apn-e-06-0-UI s1 Mus musculus cDNA, 3 end/clone = UI-M-BH2.1-apn-e-06-0-UI/ | P | 2084.9 | P | 2566 | P | 2790.5 | P |
| | | clone_end = 3/gb = AW124052/gi = 6099582/ug = Mm 21183/len = 246/STRA = for | | | | | | | |
| 3 | 93780_at | Cluster Incl AW060827 UI-M-BH1-amx-b-12-0-UI s1 Mus musculus cDNA, 3 end/clone = UI-M-BH1-amx-b-12-0-UI/ | P | 478.7 | P | 479.4 | P | 629.7 | P |
| | | clone_end = 3/gb = AW060827/gi = 6008578/ug = Mm 2125/len = 567/STRA = for | | | | | | | |
| 3 | 93966_at | Cluster Incl AI849706 UI-M-AL1-ahl-h-11-0-UI s1 Mus musculus cDNA, 3 end/clone = UI-M-AL1-ahl-h-11-0-UI/ | P | 729.1 | P | 837.5 | P | 958.2 | P |
| | | clone_end = 3/gb = AI849706/gi = 5493612/ug = Mm 21634/len = 217/STRA = for | | | | | | | |
| 3 | 94022_at | Cluster Incl AI844754 UI-M-AL1-ahq-e-04-0-UI s1 Mus musculus cDNA, 3 end/clone = UI-M-AL1-ahq-e-04-0-UI/ | P | 1062.9 | P | 1574.3 | P | 1899.8 | P |
| | | clone_end = 3/gb = AI844754/gi = 5488660/ug = Mm 21859/len = 290/STRA = for | | | | | | | |
| 3 | 94244_at | Cluster Incl AW123807: UI-M-BH2.1-apm-f-02-0-UI s1 Mus musculus cDNA, 3 end/clone = UI-M-BH2.1-apm-f-02-0-UI/ | P | 569.4 | P | 560.8 | P | 993.6 | P |
| | | clone_end = 3/gb = AW123807/gi = 6099337/ug = Mm 22360/len = 226/STRA = for | | | | | | | |
| 3 | 94248_at | Cluster Incl M62419 Mouse clathrin-associated protein (AP47) mRNA, complete cds/cds = (72,1343)/gb = M62419/ | P | 722.4 | P | 705.5 | P | 872.6 | P |
| | | gi = 191985/ug = Mm 2237/len = 1966/STRA = for | | | | | | | |
| 3 | 94290_at | Cluster Incl AW124346: UI-M-BH2.1-apq-g-05-0-UI s1 Mus musculus cDNA, 3 end/clone = UI-M-BH2.1-apq-g-05-0-UI/ | P | 413.7 | P | 416.4 | P | 566.4 | P |
| | | clone_end = 3/gb = AW124346/gi = 6099876/ug = Mm 22577/len = 235/STRA = for | | | | | | | |
| 3 | 94833_at | Cluster Incl M91380: Follistatin-like/cds = (79,999)/gb = M91380/gi = 349005/ug = Mm 22763/len = 2823/STRA = for | P | 1840.8 | P | 1386.8 | P | 2744.5 | P |
| 3 | 94842_at | Cluster Incl AI853630 UI-M-BH0-ajp-h-03-0-UI s1 Mus musculus cDNA, 3 end/clone = UI-M-BH0-ajp-h-03-0-UI/ | P | 2101.7 | P | 2116.6 | P | 2537.3 | P |
| | | clone_end = 3/gb = AI853630/gi = 5497536/ug = Mm 22876/len = 547/STRA = for | | | | | | | |
| 3 | 94920_at | Cluster Incl AI854028: UI-M-BH0-ajm-o-01-UI.s1 Mus musculus cDNA, 3 end/clone = UI-M-BH0-ajm-c-01-0-UI/ | P | 292.1 | P | 356.9 | P | 519.6 | P |
| | | clone_end = 3/gb = AI854028 = 5497934/ug = Mm.24187/len = 439/STRA = for | | | | | | | |
| 3 | 95449_at | Cluster Incl AW049793: UI-M-BH1-anm-f-04-0-UI.s1 Mus musculus cDNA, 3 end/clone = UI-M-BH1-anm-f-04-0-UI/ | P | 438.6 | P | 429.6 | P | 485.7 | P |
| | | clone_end = 3/gb = AW049793/gi = 5910322/ug = Mm.24635/len = 569/STRA = for | | | | | | | |
| 3 | 95518_at | Cluster Incl AW122893: UI-M-BH2-apa-d-07-0-UI s1 Mus musculus cDNA, 3 end/clone = UI-M-BH2.1-apa-d-07-0-UI/ | P | 1026.4 | P | 1078.5 | P | 2576.4 | P |
| | | clone_end = 3/gb = AW122893/gi = 6098423/ug = Mm 25311/len = 217/STRA = for | | | | | | | |
| 3 | 96037_at | Cluster Incl X61454 M. musculus mRNA for I3 clone/cds = (0,224)/gb = X61454/gi = 51477/ug = Mm 2585/len = 449/STRA = for | P | 875.3 | P | 917.5 | P | 1348.5 | P |
| 3 | 96082_at | Cluster Incl AI850644 UI-M-BG1-aik-f-01-0-UI s1 Mus musculus cDNA, 3 end/clone = UI-M-BG1-aik-f-01-0-UI/ | P | 834.2 | P | 918.8 | P | 1139.1 | P |
| | | clone_end = 3/gb = AI850644/gi = 5494550/ug = Mm 26614/len = 374/STRA = for | | | | | | | |
| 3 | 96614_at | Cluster Incl AA783368 vw52e04 r1 Mus musculus cDNA, 5 end/clone = IMAGE-1247550/clone_end = 5/gb = AA763388/ | P | 239.7 | P | 202.6 | P | 356 | P |
| | | gi = 2813115/ug = Mm 27112/len = 450/STRA = for | | | | | | | |
| 3 | 96615_at | Cluster Incl AI840137 UI-M-AO0-acc-b-01-UI s1 Mus musculus cDNA, 3 end/clone = UI-M-AO0-acc-b-07-0-UI/ | P | 963 | P | 850.8 | P | 1319.4 | P |
| | | clone_end = 3/gb = AI840137/gi = 5474350/ug = Mm 27114/len = 390/STRA = for | | | | | | | |
| 3 | 96662_at | Cluster Incl AI847054 UI-M-AP1-agn-a-07-0-UI s1 Mus musculus cDNA, 3 end/clone = UI-M-AP1-agn-a-07-0-UI/ | P | 370.4 | P | 327.7 | P | 477 | P |
| | | clone_end = 3/gb = AI847054/gi = 5490960/ug = Mm 27363/len = 389/STRA = for | | | | | | | |
| 3 | 97235_f_at | Cluster Incl AW124988 UI-M-BH2 1-apv-d-12-0-UI s1 Mus musculus cDNA, 3 end/clone = UI-M-BH2.1-apv-d-12-0-UI/ | P | 245.5 | P | 236.1 | P | 799.5 | P |
| | | clone_end = 3/gb = AW124988/gi = 6100518/ug = Mm 27822/len = 353/STRA = for | | | | | | | |
| 3 | 97800_at | Cluster Incl AI837308 UI-M-AK0-adf-a-09-0-UI s1 Mus musculus cDNA, 3 end/clone = UI-M-AK0-adf-a-09-0-UI/ | P | 1267.4 | P | 1287.9 | P | 1815.5 | P |
| | | clone_end = 3/gb = AI837308/gi = 5471521/ug = Mm.28123/len = 287/STRA = for | | | | | | | |
| 3 | 98908_at | Cluster Incl AW125510 UI-M-BH2-aqm-e-12-0-UI s1 Mus musculus cDNA, 3 end/clone = UI-M-BH2-aqm-e-12-0-UI/ | P | 991.4 | P | 1114.1 | P | 1618 | P |
| | | clone_end = 3/gb = AW125510/gi = 6101040/ug = Mm 28586/len = 389/STRA = for | | | | | | | |
| 3 | 94501_at | Cluster Incl AI835784 UI-M-AI0-aaq-b-10-0-UI s1 Mus musculus cDNA, 3 end/clone = UI-M-AI0-aaq-b-10-0-UI/ | P | 1562.9 | P | 1315.9 | P | 2039.3 | P |
| | | clone_end = 3/gb = AI835784/gi = 5469997/ug = Mm 28948/len = 395/STRA = for | | | | | | | |
| 3 | 95084_f_at | Cluster Incl AI844995 UI-M-BG0-ahu-e-03-0-UI s1 Mus musculus cDNA, 3 end/clone = UI-M-BG0-ahu-e-03-0-UI/ | P | 381.4 | P | 506.4 | P | 574.3 | P |
| | | clone_end = 3/gb = AI844995/gi = 5488901/ug = Mm 29256/len = 218/STRA = for | | | | | | | |
| 3 | 95117_at | Cluster Incl U04710 Insulin-like growth factor 2 receptor/cds = (132,7580)/gb = U04710/gi = 451552/ug = Mm 2938/len = 8877/ | P | 205.7 | P | 71.6 | P | 359.7 | P |
| | | STRA = for | | | | | | | |
| 3 | 95139_at | Cluster Incl AW122075 UI-M-BH2-aoo-f-04-0-UI s1 Mus musculus cDNA, 3 end/clone = UI-M-BH2-aoo-f-04-0-UI/ | P | 296.8 | P | 296.7 | P | 553.5 | P |
| | | clone_end = 3/gb = AW122075/gi = 6097482/ug = Mm 29432/len = 463/STRA = for | | | | | | | |
| 3 | 95146_at | Cluster Incl AI839280: UI-M-AO0-acb-b-06-UI s1 Mus musculus cDNA, 3 end/clone = UI-M-AO0-acb-b-06-0-UI/ | P | 440.2 | P | 467.5 | P | 637.6 | P |
| | | clone_end = 3/gb = AI839280/gi = 5473493/ug = Mm 29455/len = 265/STRA = for | | | | | | | |
| 3 | 95683_g_at | Cluster Incl AB026432 Mus musculus mRNA for XPE UV-damaged DNA binding factor, complete cds/cds = (81,3503)/ | P | 1304.8 | P | 1769.2 | P | 2416.1 | P |
| | | gb = AB026432/gi = 5931595/ug = Mm.29623/len = 4171/STRA = for | | | | | | | |
| 3 | 96255_at | Cluster Incl AF067395 Mus musculus NIX (Nix) mRNA, nuclear gene encoding mitochondrial protein, complete cds/ | P | 885.7 | P | 826.5 | P | 1249.8 | P |
| | | cds = (99,755)/gb = AF067395/gi = 138823/ug = Mm 29820/len = 1285/STRA = for | | | | | | | |
| 3 | 96267_at | Cluster Incl AI846127 UI-M-AN1-afj-b-05-0-UI s2 Mus musculus cDNA, 3 end/clone = UI-M-AN1-afj-b-05-0-UI/ | P | 2923.1 | P | 3399 | P | 3744.7 | P |
| | | clone_end = 3/gb = AI846127/gi = 5490033/ug = Mm 29842/len = 417/STRA = for | | | | | | | |
| 3 | 96344_at | Cluster Incl X61600 M musculus gene for beta-enolase/cds = (66,1370)/gb = X61600/gi = 50848/ug = Mm 29994/len = 1450/ | P | 2518.5 | P | 3696.3 | P | 4601.2 | P |
| | | STRA = for | | | | | | | |
| 3 | 96351_at | Cluster Incl AI846097 UI-M-AK1-aew-c-06-0-UI s1 Mus musculus cDNA, 3 end/clone = UI-M-AK1-aew-c-06-0-UI/ | P | 382.1 | P | 212.9 | P | 604.9 | P |
| | | clone_end = 3/gb = AI846097/gi = 5489968/ug = Mm 30001/len = 183/STRA = for | | | | | | | |
| 3 | 96854_at | Cluster Incl AJ010391 Mus musculus copa gene, partial/cds = (0,94)/gb = AJ010391/gi = 5763780/ug = Mm 30041/len = 95/ | P | 842.3 | P | 1016.4 | P | 1302.6 | P |
| | | STRA = for | | | | | | | |
| 3 | 96916_at | Cluster Incl AW046793 UI-M-BH1-alz-b-05-0-UI.s1 Mus musculus cDNA, 3 end/clone = UI-M-BH1-alz-b-05-0-UI/ | P | 585.1 | P | 737.7 | P | 953 | P |
| | | clone_end = 3/gb = AW046793/gi = 5907322/ug = Mm 30152/len = 237/STRA = for | | | | | | | |
| 3 | 96954_at | Cluster Incl AW122567 UI-M-BH2-2-aox-f-01-0-UI s1 Mus musculus cDNA, 3 end/clone = UI-M-BH2 2-aox-f-01-0-UI/ | P | 383.4 | P | 586.7 | P | 726.5 | P |
| | | clone_end = 3/gb = AW122567/gi = 6098092/ug = Mm 30212/len = 391/STRA = for | | | | | | | |
| 3 | 96956_at | Cluster Incl AW125526 UI-M-BH2.2-aqm-g-04-0-UI.s1 Mus musculus cDNA, 3 end/clone = UI-M-BH2.2-aqm-g-04-0-UI/ | P | 2119.8 | P | 2221.9 | P | 2941.8 | P |
| | | clone_end = 3/gb = AW125526/gi = 6101058/ug = Mm 30215/len = 443/STRA = for | | | | | | | |
| 3 | 97447_at | Cluster Incl AI836718 UI-M-AJ0-aax-d-11-0-UI.s1 Mus musculus cDNA, 3 end/clone = UI-M-AJ0-aax-d-11-0-UI/ | P | 841.5 | P | 1186.6 | P | 1565.6 | P |
| | | clone_end = 3/gb = AI836718/gi = 5470931/ug = Mm 30244/len = 276/STRA = for | | | | | | | |
| 3 | 97509_f_at | Cluster Incl U22324 Mus musculus fibroblast growth factor receptor-1 mRNA, long isoform precursor, complete cds/ | P | 505.9 | P | 597.5 | P | 721.8 | P |
| | | cds = (57,2525)/gb = U22324/gi = 722340/ug = Mm 3157/len = 2526/STRA = for | | | | | | | |
| 3 | 97526_at | Cluster Incl AW123294: UI-M-BH2 1-apg-h-02-0-UI s1 Mus musculus cDNA, 3 end/clone = UI-M-BH2 1-apg-h-02-0-UI/ | P | 315.6 | P | 343 | P | 562.2 | P |
| | | clone_end = 3/gb = AW123294/gi = 6098824/ug = Mm 3256/len = 380/STRA = for | | | | | | | |
| 3 | 98042_at | Cluster Incl AW049787 UI-M-BH1-anm-e-03-0-UI s1 Mus musculus cDNA, 3 end/clone = UI-M-BH1-anm-e-03-0-UI/ | P | 399.5 | P | 417.5 | P | 806.4 | P |
| | | clone_end = 3/gb = AW049787/gi = 5910316/ug = Mm 34126/len = 434/STRA = for | | | | | | | |
| 3 | 98097_r_at | Cluster Incl AI840219: UI-M-AI0-aal-g-03-0-UI s2 Mus musculus cDNA, 3 end/clone = UI-M-Al0-aal-g-03-0-UI/ | P | 103.8 | P | 115.7 | P | 175.1 | P |
| | | gb = AI840219/gi = 5474432/ug = Mm.34708/len = 371/STRA = for | | | | | | | |
| 3 | 98126_s_at | Cluster Incl X67140: Atpase, Ca++ transporting, cardiac muscle, fast twitch 1/cds = (0,548)/gb = X67140/gi = 509146/ | P | 1500 | P | 3077.2 | P | 5227.6 | P |
| | | ug = Mm 35134/len = 855/STRA = for | | | | | | | |
| 3 | 98518_f_at | Cluster Incl AI604345 vv70f10.x1 Mus musculus cDNA, 3 end/clone = IMAGE-1227787/clone_end = 3/gb = AI604345/ | P | 4571.8 | P | 5324 | P | 7141.6 | P |
| | | gi = 4613512/ug = Mm.35806/len = 397/STRA = for | | | | | | | |
| 3 | 98519_r_at | Cluster Incl AI604345 vv70f10 x1 Mus musculus cDNA, 3 end/clone = IMAGE-1227787/clone_end = 3/gb = AI604345/ | P | 1024.8 | P | 731.5 | P | 2159.9 | P |
| | | gi = 4613512/ug = Mm.35806/len = 397/STRA = for | | | | | | | |
| 3 | 98624_at | Cluster Incl X75316 M musculus seb4 mRNA/cds = (235,948)/gb = X75316/gi = 407467/ug = Mm.3865/len = 1908/STRA = for | P | 235.8 | P | 382.7 | P | 526.5 | P |
| 3 | 98630_at | Cluster Incl AW106745 um33b04 y1 Mus musculus cDNA, 5 end/clone = IMAGE-2236303/clone_end = 5/gb = AW106745/ | P | 355.7 | P | 328.4 | P | 578.1 | P |
| | | gi = 6077545/ug = Mm 38792/len = 600/STRA = for | | | | | | | |
| 3 | 99103_at | Cluster Incl AF036341 Mus musculus interferon regulatory factor-3 (IRF3) gene, partial cds/cds = (0,247)/gb = AF036341/ | P | 218.5 | P | 289.7 | P | 328.5 | P |
| | | gi = 2674204/ug = Mm 3960/len = 248/STRA = for | | | | | | | |
| 3 | 99142_at | Cluster Incl AI834895: UI-M-ANI-afe-d-08-0-UI.s1 Mus musculus cDNA, 3 end/clone = UI-M-AN1-afe-d-08-0-UI/ | P | 508.2 | P | 718.5 | P | 892.8 | P |
| | | clone_end = 3/gb = AI834895/gi = 5469108/ug = Mm 41405/len = 436/STRA = for | | | | | | | |
| 3 | 99153_at | Cluster Incl AI854607: UI-M-BH0-ake-c-10-0-UI.s1 Mus musculus cDNA, 3 end/clone = UI-M-BH0-ake-c-10-0-UI/ | P | 251.4 | P | 218.2 | P | 360.4 | P |
| | | clone_end = 3/gb = AI854607/gi = 5498513/ug = Mm 41574/len = 489/STRA = for | | | | | | | |
| 3 | 99161_at | Cluster Incl AI843359: UI-M-AQ1-adz-d-05-0-UI s1 Mus musculus cDNA, 3 end/clone = UI-M-AQ1-adz-d-05-0-UI/ | P | 1482.5 | P | 1345.8 | P | 2111.4 | P |
| | | clone_end = 3/gb = AI843359/gi = 5477572/ug = Mm 41665/len = 425/STRA = for | | | | | | | |
| 3 | 99188_at | Cluster Incl AA615161 vn96a04 r1 Mus musculus cDNA, 5 end/clone = IMAGE-1039758/clone_end = 5/gb = AA615161/ | P | 611.5 | P | 901.1 | P | 1115.3 | P |
| | | gi = 2502389/ug = Mm 41890/len = 495/STRA = for | | | | | | | |
| 3 | 99667_at | Cluster Incl U08439 Mus musculus Balb/c cytochrome c oxidase subunit VIaH mRNA, complete cds/cds = (57,350)/ | P | 658.3 | P | 957.4 | P | 1507 | P |
| | | gb = U08439/gi = 475704/ug = Mm.43824/len = 466/STRA = for | | | | | | | |
| 3 | 99675_at | Cluster Incl AI839766 UI-M-AN0-acq-e-09-0-UI.s1 Mus musculus cDNA, 3 end/clone = UI-M-AN0-acq-e-09-0-UI/ | P | 280.7 | P | 281 | P | 631.4 | P |
| | | clone_end = 3/gb = AI839766/gi = 5474019/ug = Mm 44202/len = 366/STRA = for | | | | | | | |
| 3 | 100057_at | Cluster Incl AI838320 UI-M-AO0-aby-f-05-0-UI s1 Mus musculus cDNA, 3 end/clone = UI-M-AO0-aby-f-05-0-UI/ | P | 246.5 | P | 283.7 | P | 483.7 | P |
| | | clone_end = 3/gb = AI838320/gi = 5472533/ug = Mm 4467/len = 529/STRA = for | | | | | | | |
| 3 | 100084_at | Cluster Incl X60671 M musculus mRNA for ezrin/cds = (95,1855)/gb = X60671/gi = 50880/ug = Mm 4451/len = 2701/ | P | 870.5 | P | 1264.1 | P | 1361.3 | P |
| | | STRA = for | | | | | | | |
| 3 | 100571_at | Cluster Incl AW123934 UI-M-BH2.3-aqc-b-11-0-UI s1 Mus musculus cDNA, 3 end/clone = UI-M-BH2 3-aqc-b-11-0-UI/ | P | 608.3 | P | 674.5 | P | 946.2 | P |
| | | clone_end = 3/gb = AW123934/gi = 6099464/ug = Mm 5856/len = 237/STRA = for | | | | | | | |
| 3 | 100574_f_at | Cluster Incl L09104 Glucose phosphate isomerase 1 complex/cds = (2066,2938)/gb = L09104/gi = 309267/ug = Mm 589/ | P | 3162.7 | P | 4308.7 | P | 5033.7 | P |
| | | len = 3194/STRA = for | | | | | | | |
| 3 | 100600_at | Cluster Incl M58661 CD24a antigen/cds = (72,302)/gb = M58661/gi = 198985/ug = Mm 6417/len = 1800/STRA = for | P | 3321.8 | P | 3884 | P | 4365.6 | P |
| 3 | 100630_f_at | Cluster Incl AA241764.mw26g09 r1 Mus musculus cDNA, 5 end/clone = IMAGE-671872/clone_end = 5/gb = AA241764/ | P | 1452.8 | P | 1957.5 | P | 2356.3 | P |
| | | gi = 1872796/ug = Mm 667/len = 240/STRA = for | | | | | | | |
| 3 | 101029_f_at | Cluster Incl M15501 Actin, alpha, cardiac/cds = (0,1127)/gb = M15501/gi = 191653/ug = Mm 686/len = 1226/STRA = for | P | 2880.6 | P | 4075.4 | P | 5273.8 | P |
| 3 | 101047_at | Cluster Incl AW123697 UI-M-BH2 1-apl-c-11-0-UI s1 Mus musculus cDNA, 3 end/clone = UI-M-BH2 1-apl-c-11-0-UI/ | P | 1487.4 | P | 1841.1 | P | 2558.6 | P |
| | | clone_end = 3/gb = AW123697/gi = 6099227/ug = Mm.7/len = 413/STRA = for | | | | | | | |
| 3 | 101055_at | Cluster Incl J05261 Protective protein for beta-galactosidase/cds = (213,1637)/gb = J05261/gi = 200492/ug = Mm 7046/ | P | 1048.1 | P | 1083.8 | P | 1556.7 | P |
| | | len = 1987/STRA = for | | | | | | | |
| 3 | 101102_at | Cluster Incl AJ223156 Mus musculus alpha4 gene, exon2 and join CDS/cds = (0,831)/gb = AJ223156/gi = 3127824/ | P | 440.8 | P | 454.1 | P | 696 | P |
| | | ug = Mm 7454/len = 1132/STRA = for | | | | | | | |
| 3 | 101109_at | Cluster Incl U43512 Dystroglycan 1/cds = (0,825)/gb = U43512/gi = 1155350/ug = Mm 7524/len = 3170/STRA = for | P | 1235.3 | P | 1135.9 | P | 1594.4 | P |
| 3 | 101517_at | Cluster Incl X81058: M musculus tex261 mRNA/cds = (35,625)/gb = X81058/gi = 531527/ug = Mm 848/len = 1398/STRA = for | P | 610 | P | 692.8 | P | 900.8 | P |
| 3 | 101973_at | Cluster Incl Y15163 Mus musculus mRNA for mrg1 protein/cds = (219,1028)/gb = Y15163/gi = 3059126/ug = Mm.9524/ | P | 667.6 | P | 452.2 | P | 883.8 | P |
| | | len = 1943/STRA = for | | | | | | | |
| 3 | 101995_at | Cluster Incl U40930 Mus musculus oxidative stress-induced protein mRNA, complete cds/cds = (33,1361)/gb = U40930/ | P | 765.2 | P | 769.5 | P | 1298.9 | P |
| | | gi = 1280523/ug = Mm 9846/len = 2000/STRA = for | | | | | | | |
| 3 | 102024_at | Cluster Incl AF000581 Mus musculus p300/CBP/Co-Integrator protein mRNA, complete cds/cds = (109,4305)/ | P | 65.4 | P | 76.2 | P | 152.6 | P |
| | | gb = AF000581/gi = 2934905/ug = Mm 1011/len = 4609/STRA = for | | | | | | | |
| 3 | 102043_at | Cluster Incl AF047704 Mus musculus tuftelin mRNA, complete cds/cds = (22,1194)/gb = AF047704/gi = 2920655/ | P | 664.2 | P | 397.2 | P | 959.9 | P |
| | | ug = Mm 10214/len = 2580/STRA = for | | | | | | | |
| 3 | 102063_at | Cluster Incl AF079535: Mus musculus 3-phosphoinositide dependent protein kinase-1 mRNA, complete cds/cds = (91,1770)/ | P | 166.8 | P | 248.4 | P | 337.5 | P |
| | | gb = AF079535/gi = 4028126/ug = Mm 10504/len = 1923/STRA = for | | | | | | | |
| 3 | 102336_at | Cluster Incl AF060565: Mus musculus RW1 protein mRNA, complete cds/cds = (28,5517)/gb = AF060565/gi = 3091277/ | P | 688.4 | P | 637.7 | P | 699.3 | P |
| | | ug = Mm 10808/len = 6271/STRA = for | | | | | | | |
| 3 | 102382_at | Cluster Incl AB014494: Aryl hydrocarbon receptor nuclear translocator-like/cds = (406,2286)/gb = AB014494/gi = 3402482/ | P | 129.9 | P | 85.4 | P | 262 | P |
| | | ug = Mm.12177/len = 2792/STRA = for | | | | | | | |
| 3 | 102402_at | Cluster Incl AJ001261 Mus musculus mRNA for NIPSNAP2 protein/cds = (48,893)/gb = AJ001261/gi = 2769257/ | P | 926.6 | P | 961.9 | P | 1421.8 | P |
| | | ug = Mm.12468/len = 1604/STRA = for | | | | | | | |
| 3 | 102413_at | Cluster Incl AW124311 UI-M-BH2.1-apq-d-04-0-UI.s1 Mus musculus cDNA, 3 end/clone = UI-M-BH2.1-apq-d-04-0-UI/ | P | 816.8 | P | 1023.7 | P | 1273.9 | P |
| | | clone_end = 3/gb = AW124311/gi = 6099841/ug = Mm 12607/len = 222/STRA = for | | | | | | | |
| 3 | 102426_at | Cluster Incl U93291 Mus musculus skeletal muscle calsequestrin mRNA, complete cds/cds = (179,1378)/gb = U93291/ | P | 847.6 | P | 1797 | P | 2227.1 | P |
| | | gi = 1931626/ug = Mm 12829/len = 1395/STRA = for | | | | | | | |
| 3 | 102797_at | Cluster Incl X95281: Mus musculus mRNA for retinal short-chain dehydrogenase/reductase/cds = (416,1324)/gb = X95281/ | P | 317.6 | P | 365.1 | P | 405.7 | P |
| | | gi = 3776074/ug = Mm 14063/len = 1754/STRA = for | | | | | | | |
| 3 | 103094_at | Cluster Incl AI036894 uh20g09.r1 Mus musculus cDNA, 5 end/clone = IMAGE-1746016/clone_end = 5/gb = AI036894/ | P | 386 | P | 479.5 | P | 757.1 | P |
| | | gi = 3260585/ug = Mm.1749/len = 163/STRA = for | | | | | | | |
| 3 | 103322_at | Cluster Incl L35034 Ribose 5-phosphate isomerase A/cds = (207,917)/gb = L35034/gi = 836673/ug = Mm 17905/len = 1763/ | P | 890.8 | P | 651.1 | P | 1039.9 | P |
| | | STRA = for | | | | | | | |
| 3 | 103412_at | Cluster Incl AI847230.UI-M-AI1-afq-0-12-0-UI s1 Mus musculus CDNA, 3 end/clone = UI-M-AI1-afq-c-12-0-UI/clone_end = 3/ | P | 199.5 | P | 207.9 | P | 357.2 | P |
| | | gb = AI847230/gi = 5491136/ug = Mm 18830/len = 459/STRA = for | | | | | | | |
| 3 | 103581_at | Cluster Incl Y14004.Mus musculus mRNA for acyl-CoA thioesterase/cds = (8,1267)/gb = Y14004/gi = 2765361/ug = Mm.1978/ | P | 530.5 | P | 610.6 | P | 789.7 | P |
| | | len = 1574/STRA = for | | | | | | | |
| 3 | 103623_at | Cluster Incl L39790 Mus musculus fibrillin 2 (fbn2) gene, complete cds/cds = (0,8723)/gb = L39790/gi = 762830/ | P | 259.8 | P | 459.8 | P | 885.3 | P |
| | | ug = Mm 20271/len = 8724/STRA = for | | | | | | | |
| 3 | 103644_at | Cluster Incl D13139.Dipeptidase 1 (renal)/cds = (257,1489)/gb = D13139/gi = 220397/ug = Mm 20388/len = 1655/STRA = for | P | 238.1 | P | 244.1 | P | 498.7 | P |
| 3 | 103684_f_at | Cluster Incl AA959648 vw57102 s1 Mus musculus cDNA, 3 end/clone = IMAGE-1247931/clone_end = 3/gb = AA959648/ | P | 229.6 | P | 278.1 | P | 350 | P |
| | | gi = 3125548/ug = Mm.20693/len = 426/STRA = for | | | | | | | |
| 3 | 103675_at | Cluster Incl Y17793 Mus musculus mRNA for Dutt1 protein/cds = (253,5091)/gb = Y17793/gi = 3688435/ug = Mm.20832/ | P | 95.9 | P | 127.2 | P | 194 | P |
| | | len = 6922/STRA = for | | | | | | | |
| 3 | 103807_at | Cluster Incl AB012265: Mus musculus mRNA for wizl, complete cds/cds = (772,5457)/gb = AB012265/gi = 3551181/ | P | 280.2 | P | 303.6 | P | 537.8 | P |
| | | ug = Mm.20878/len = 6425/STRA = rev | | | | | | | |
| 3 | 103809_r_at | Cluster Incl AF063229: Dynein, cyctoplasmic, intermediate chain 1/cds = (141,2027)/gb = AF063229/gi = 3493586/ | P | 824.4 | P | 883.8 | P | 1041.1 | P |
| | | ug = Mm 20893/len = 2586/STRA = for | | | | | | | |
| 3 | 103826_at | Cluster Incl AF086830 Mus musculus leukemia/lymphoma related factor LRF (Lrf) mRNA, complete cds/cds = (58,1755)/ | P | 566.2 | P | 760.4 | P | 935.9 | P |
| | | gb = AF086830/gi = 3599510/ug = Mm 20920/len = 1793/STRA = for | | | | | | | |
| 3 | 103904_at | Cluster Incl X81584 M musculus mRNA for Insulin-like growth factor binding protein-6/cds = (192,908)/gb = X81584/ | P | 438.6 | P | 456.5 | P | 1289.8 | P |
| | | gi = 550386/ug = Mm.21373/len = 1138/STRA = for | | | | | | | |
| 3 | 104044_at | Cluster Incl AI853663 UI-M-BH0-ajq-f-08-0-UI s1 Mus musculus cDNA, 3 end/clone = UI-M-BH0-ajq-f-08-0-UI/ | P | 524.3 | P | 658.3 | P | 756.2 | P |
| | | clone_end = 3/gb = AI853663/gi = 5497609/ug = Mm.21480/len = 432/STRA = for | | | | | | | |
| 3 | 104059_at | Cluster Incl AI851751 UI-M-BH0-aim-c-03-0-UI.s1 Mus musculus cDNA, 3 end/clone = UI-M-BH0-aim-c-03-0-UI/ | P | 309.9 | P | 412.8 | P | 500.7 | P |
| | | clone_end = 3/gb = AI851751/gi = 5495657/ug = Mm.21553/len = 377/STRA = for | | | | | | | |
| 3 | 104082_at | Cluster Incl AI848325 UI-M-AH1-ago-b-05-0-UI s1 Mus musculus cDNA, 3 end/clone = UI-M-AH1-ago-b-05-0-UI | P | 472.9 | P | 638.6 | P | 872.6 | P |
| | | clone_end = 3/gb = AI848325/gi = 5492231/ug = Mm 21759/len = 559/STRA = for | | | | | | | |
| 3 | 104140_s_at | Cluster Incl AI846989 UI-M-AP1-agi-b-11-0-UI s1 Mus musculus cDNA, 3 end/clone = UI-M-AP1-agi-b-11-0-UI/ | P | 741 | P | 1051.7 | P | 1553.3 | P |
| | | clone_end = 3/gb = AI846989/gi = 5490895/ug = Mm.22121/len = 137/STRA = for | | | | | | | |
| 3 | 104141_at | Cluster Incl AI850401 UI-M-BG1-aig-f-01-0-UI s1 Mus musculus cDNA, 3 end/clone = UI-M-BG1-aig-f-01-0-UI/ | P | 625.5 | P | 696.8 | P | 829 | P |
| | | clone_end = 3/gb = AI850401/gi = 5494307/ug = Mm.22123/len = 511/STRA = for | | | | | | | |
| 3 | 104148_at | Cluster Incl AA939571: vz51d08 r1 Mus musculus cDNA, 5 end/clone = IMAGE.1329999/clone_end = 5/gb = AA939571/ | P | 589.3 | P | 771.9 | P | 852.7 | P |
| | | gi = 3100348/ug = Mm 22183/len = 420/STRA = for | | | | | | | |
| 3 | 104314_at | Cluster Incl AI851206 UI-M-BH0-aix-f-05-0-UI s1 Mus musculus cDNA, 3 end/clone = UI-M-BH0-ajx-f-05-0-UI/clone_end = 3/ | P | 674.9 | P | 816.2 | P | 1035.6 | P |
| | | gb = AI851206/gi = 5495112/ug = Mm.22405/len = 405/STRA = for | | | | | | | |
| 3 | 104331_at | Cluster Incl AA832935: uc93c01 r1 Mus musculus cDNA, 5 end/clone = IMAGE-1433184/clone_end = 5/gb = AA832935/ | P | 1107.4 | P | 1268.9 | P | 1296.5 | P |
| | | gi = 2906863/ug = Mm.22478/len = 391/STRA = for | | | | | | | |
| 3 | 104360_at | Cluster Incl AI841844 UI-M-AO0-acb-g-01-0-UI s2 Mus musculus cDNA, 3 end/clone = UI-M-AO0-acb-g-01-0-UI/ | P | 1433.1 | P | 1350.5 | P | 1770 | P |
| | | clone_end = 3/gb = AI841844/gi = 5476057/ug = Mm.22588/len = 507/STRA = for | | | | | | | |
| 3 | 104363_at | Cluster Incl AA763213: vv88f01.r1 Mus musculus cDNA, 5 end/clone = IMAGE-1229497/clone_end = 5/gb = AA763213/ | P | 2832.2 | P | 2949.1 | P | 4605.8 | P |
| | | gi = 2812960/ug = Mm.22608/len = 309/STRA = rev | | | | | | | |
| 3 | 104366_at | Cluster Incl AW047831 UI-M-BH1-ais-c-04-0-UI s1 Mus musculus cDNA, 3 end/clone = UI-M-BH1-als-c-04-0-UI/ | P | 437.6 | P | 457.1 | P | 658.7 | P |
| | | clone_end = 3/gb = AW047831/gi = 5908360/ug = Mm.22616/len = 200/STRA = for | | | | | | | |
| 3 | 104555_at | Cluster Incl AA733946: vv21f05 r1 Mus musculus cDNA, 5 end/clone = IMAGE-1223073/clone_end = 5/gb = AA733946/ | P | 828.7 | P | 1413.6 | P | 2394.4 | P |
| | | gi = 2755613/ug = Mm.23786/len = 313/STRA = for | | | | | | | |
| 3 | 104607_at | Cluster Incl AF093624 Mus musculus Nap-like 1 protein (Nspl 1) gene, complete cds, tRNA-Sec gene, complete sequence, | P | 262 | P | 459.9 | P | 872 | P |
| | | and FosB protein (Fosb) gene, complete cds/cds = (164,1579)/gb = AF093624/gi = 4235385/ug = Mm.24142/len = 1938/ | | | | | | | |
| | | STRA = for | | | | | | | |
| 3 | 104614_at | Cluster Incl AI852765 UI-M-BH0-aly-c-05-0-UI s1 Mus musculus cDNA, 3 end/clone = UI-M-BH0-aly-c-05-0-UI/ | P | 886 | P | 1231.6 | P | 1848.2 | P |
| | | clone_end = 3/gb = AI852765/gi = 5496671/ug = Mm.24193/len = 401/STRA = for | | | | | | | |
| 3 | 104709_at | Cluster Incl AI843665 UI-M-AK1-aer-a-11-0-UI s1 Mus musculus cDNA, 3 end/clone = UI-M-AK1-aer-a-11-0-UI/ | P | 542.1 | P | 452.9 | P | 757.2 | P |
| | | clone_end = 3/gb = AI843665/gi = 5477878/ug = Mm.24426/len = 314/STRA = for | | | | | | | |
| 3 | 104760_at | Cluster Incl AI838527 UI-M-AL0-abp-a-12-0-UI s1 Mus musculus cDNA, 3 end/clone = UI-M-AL0-abp-a-12-0-UI/ | P | 550.7 | P | 566.9 | P | 956.9 | P |
| | | clone_end = 3/gb = AI838527/gi = 5472705/ug = Mm.24831/len = 305/STRA = for | | | | | | | |
| 3 | 94438_at | Cluster Incl AI852672 UI-M-BH0-aji-b-09-0-UI s1 Mus musculus cDNA, 3 end/clone = UI-M-BH0-aji-b-09-0-UI/clone_end = 3/ | P | 583.6 | P | 765.7 | P | 1599.7 | P |
| | | gb = AI852672/gi = 5496578/ug = Mm 26550/len = 381/STRA = for | | | | | | | |
| 3 | 94952_at | Cluster Incl AI152780 ud91a10 rt Mus musculus cDNA, 5 end/clone = IMAGE-1478202/clone_end = 5/gb = AI152780/ | P | 894 | P | 1025.8 | P | 1335.7 | P |
| | | gi = 3681249/ug = Mm.27118/len = 250/STRA = for | | | | | | | |
| 3 | 94968_at | Cluster Incl AW048793: UI-M-BH1-amg-c-06-0-UI s1 Mus musculus cDNA, 3 end/clone = UI-M-BH1-amg-c-06-0-UI/ | P | 803.7 | P | 1006.3 | P | 1283.6 | P |
| | | clone_end = 3/gb = AW048793/gi = 5909322/ug = Mm 27220/len = 335/STRA = for | | | | | | | |
| 3 | 95531_at | Cluster Incl AI854771 UI-M-BH0-aka-c-10-0-UI s1 Mus musculus cDNA, 3 end/clone = UI-M-BH0-aka-c-10-0-UI/ | P | 231.7 | P | 364.2 | P | 538.9 | P |
| | | clone_end = 3/gb = AI854771/gi = 5498677/ug = Mm.27633/len = 519/STRA = for | | | | | | | |
| 3 | 95547_at | Cluster Incl D89821 Mus musculus mRNA for RhoM, complete cds/cds = (84,716)/gb = D89821/gi = 1752824/ug = Mm 27701/ | P | 277 | P | 270.7 | P | 382 | P |
| | | len = 1166/STRA = for | | | | | | | |
| 3 | 95587_at | Cluster Incl AI837204 UI-M-AK0-add-f-07-0-UI.s1 Mus musculus cDNA, 3 end/clone = UI-M-AK0-add-f-07-0-UI/ | P | 364.4 | P | 524.9 | P | 602.7 | P |
| | | clone_end = 3/gb = AI837204/gi = 5471417/ug = Mm.27862/len = 287/STRA = for | | | | | | | |
| 3 | 95607_at | Cluster Incl X82457 M. musculus es64 mRNA/cds = (176,1516)/gb = X82457/gi = 951299/ug = Mm.27983/len = 2054/ | P | 458 | P | 468.5 | P | 622.6 | P |
| | | STRA = for | | | | | | | |
| 3 | 95625_at | Cluster Incl AI840149 UI-M-AO0-acc-c-09-0-UI.s1 Mus musculus cDNA, 3 end/clone = UI-M-AO0-acc-c-09-UI/ | P | 304.4 | P | 418.2 | P | 597.3 | P |
| | | clone_end = 3/gb = AI840149/gi = 5474362/ug = Mm 28056/len = 339/STRA = for | | | | | | | |
| 3 | 96140_at | Cluster Incl AF039021: Mus musculus pencentriolar material 1 (Pcm1) gene, partial cds/cds = (0,196)/gb = AF039021/ | P | 165.1 | P | 222.6 | P | 295.2 | P |
| | | gi = 3170134/ug = Mm.28192/len = 197/STRA = for | | | | | | | |
| 3 | 96154_at | Cluster Incl AA600645: vm77b04 r1 Mus musculus cDNA, 5 end/clone = IMAGE-1004239/clone_end = 5/gb = AA600645/ | P | 330.2 | P | 474.4 | P | 633.1 | P |
| | | gi = 2434270/ug = Mm.28280/len = 378/STRA = for | | | | | | | |
| 3 | 96156_at | Cluster Incl AA874329 vx79h02.r1 Mus musculus cDNA, 5 end/clone = IMAGE-1281459/clone_end = 5/gb = AA874329/ | P | 277.2 | P | 269.1 | P | 282.5 | P |
| | | gi = 2979018/ug = Mm.28311/len = 206/STRA = for | | | | | | | |
| 3 | 96186_at | Cluster Incl AI839288 UI-M-AO0-acb-c-08-0-UI s1 Mus musculus cDNA, 3 end/clone = UI-M-AO0-acb-c-08-0-UI/ | P | 483.8 | P | 692.9 | P | 892.2 | P |
| | | clone_end = 3/gb = AI839286/gi = 5473499/ug = Mm.28465/len = 258/STRA = for | | | | | | | |
| 3 | 96200_at | Cluster Incl AI838344 UI-M-AO0-aby-h-11-0-UI s1 Mus musculus cDNA, 3 end/clone = UI-M-AO0-aby-h-11-0-UI/ | P | 477.2 | P | 522.9 | P | 598.9 | P |
| | | clone_end = 3/gb = AI838344/gi = 5472557/ug = Mm.28595/len = 393/STRA = for | | | | | | | |
| 3 | 96731_at | Cluster Incl AA615100: vo34f04 r1 Mus musculus cDNA, 5 end/clone = IMAGE-1051807/clone_end = 5/gb = AA615100/ | P | 518 | P | 565.9 | P | 1040.4 | P |
| | | gi = 2502328/ug = Mm 28869/len = 145/STRA = for | | | | | | | |
| 3 | 96747_at | Cluster Incl AW121294 UI-M-BH2 3-aog-d-09-0-UI s1 Mus musculus cDNA, 3 end/clone = UI-M-BH2 3-aog-d-09-0-UI/ | P | 471 | P | 579.1 | P | 704.8 | P |
| | | clone_end = 3/gb = AW121294/gi = 6096627/ug = Mm.28954/len = 235/STRA = for | | | | | | | |
| 3 | 96761_at | Cluster Incl AF109906: Mus musculus MHC class III region RD gene, partial cds; Bf, C2, G9A, NG22, G9, HSP70, HSP70, | P | 451 | P | 566.1 | P | 815.2 | P |
| | | HSC70t, and smRNP genes, complete cds; G7A gene, partial cds; and unknown genes/cds = (0,2123)/gb = AF 109906/ | | | | | | | |
| | | gi = 3986763/ug = Mm.29004/len = 212/STRA = for | | | | | | | |
| 3 | 96771_at | Cluster Incl AI1006228 ua88d09 r1 Mus musculus cDNA, 5 end/clone = IMAGE-1364561/clone_end = 5/gb = AI006228/ | P | 989.7 | P | 963.4 | P | 1413.8 | P |
| | | gi = 3215837/ug = Mm 29023/len = 382/STRA = rev | | | | | | | |
| 3 | 96837_at | Cluster Incl AI837492 UI-M-ALO-abr-f-11-0-UI s1 Mus musculus cDNA, 3 end/clone = UI-M-AL0-abr-f-11-0-UI/clone_end = 3/ | P | 1083.4 | P | 1077.3 | P | 1616.3 | P |
| | | gb = AI837492/gi = 5471705/ug = Mm.29442/len = 181/STRA = for | | | | | | | |
| 3 | 97357_at | Cluster Incl AI426400 mm50h04 x1 Mus musculus cDNA, 3 end/clone = IMAGE-524983/clone_end = 3/gb = AI426400/ | P | 179.2 | P | 168.6 | P | 239.8 | P |
| | | gi = 4272326/ug = Mm 29635/len = 157/STRA = for | | | | | | | |
| 3 | 97929_r_at | Cluster Incl AW124782: UI-M-BH2.1-aqa-h-02-0-UI s1 Mus musculus cDNA, 3 end/clone = UI-M-BH2 1-aqa-h-02-0-UI/ | P | 719.5 | P | 945.5 | P | 1040.4 | P |
| | | clone_end = 3/gb = AW124782/gi = 6100312/ug = Mm 30237/len = 484/STRA = for | | | | | | | |
| 3 | 97935_at | Cluster Incl AI842970 UI-M-AO1-aem-g-06-0-UI s1 Mus musculus cDNA, 3 end/clone = UI-M-AO1-aem-g-06-0-UI/ | P | 536.1 | P | 546.5 | P | 681.1 | P |
| | | clone_end = 3/gb = AI842970/gi = 5477183/ug = Mm 30252/len = 374/STRA = for | | | | | | | |
| 3 | 97994_at | Cluster Incl AI019193 ub20e12 r1 Mus musculus cDNA, 5 end/clone = IMAGE-1378318/clone_end = 5/gb = AI019193/ | P | 347.4 | P | 361.3 | P | 504.9 | P |
| | | gi = 3233529/ug = Mm 31630/len = 476/STRA = for | | | | | | | |
| 3 | 98027_at | Cluster Incl Z22923 Procollagen, type IX, alpha 2/cds = (383,2449)/gb = Z22923/gi = 311949/ug = Mm 3277/len = 2956/ | P | 322.3 | P | 342 | P | 524.7 | P |
| | | STRA = for | | | | | | | |
| 3 | 98426_at | Cluster Incl AF030001 Mus musculus major histocompatibility locus class III region-butyrophilin-like protein gene, partial | P | 212.8 | P | 207.8 | P | 397.8 | P |
| | | cds, Notch4, PBX2, RAGE, lysophatidic acid acyl transferase-alpha, palmitoyl-protein thioesterase 2 (PPT2), CREB-RP, | | | | | | | |
| | | and tenascin X/STRA = for | | | | | | | |
| 3 | 98435_at | Cluster Incl M74495 Mouse adenylosuccinate synthetase mRNA, complete cds/cds = (30,1403)/gb = M74495/gi = 404750/ | P | 137.7 | P | 206.5 | P | 647.8 | P |
| | | ug = Mm 3440/len = 1736/STRA = for | | | | | | | |
| 3 | 98454_at | Cluster Incl Y14771 Mus musculus mRNA for paralernmin/cds = (127,1278)/gb = Y14771/gi = 4456676/ug = Mm 34650/ | P | 458.5 | P | 813.5 | P | 731.5 | P |
| | | len = 1936/STRA = for | | | | | | | |
| 3 | 98460_at | Cluster Incl AJ237917 Mus musculus mRNA for fatso protein/cds = (37,1545)/gb = AJ237917/gi = 6102999/ug = Mm.34778/ | P | 514 | P | 613.7 | P | 675.1 | P |
| | | len = 3554/STRA = for | | | | | | | |
| 3 | 98476_at | Cluster Incl L40631 Ankyrin 3, epithelial/cds = (647,3931)/gb = L40631/gi = 710545/ug = Mm 3526/len = 5074/STRA = for | P | 235 | P | 285.5 | P | 533.4 | P |
| 3 | 98989_at | Cluster Incl AF057368 7-dehydrocholesterol reductase/cds = (39,1454)/gb = AF057368/gi = 3283173/ug = Mm 3747/ | P | 221.7 | P | 293.5 | P | 387.7 | P |
| | | len = 2377/STRA = rev | | | | | | | |
| 3 | 99009_at | Cluster Incl Z49204 Nicotinamide nucleotide transhydrogenase/cds = (68,3328)/gb = Z49204/gi = 840318/ug = Mm 3842/ | P | 310.9 | P | 279.3 | P | 677.3 | P |
| | | len = 3844/STRA = for | | | | | | | |
| 3 | 99024_at | Cluster Incl U32395 Mus musculus Max-interacting transcriptional repressor (Mad4) mRNA, complete cds/cds = (0,629)/ | P | 782.9 | P | 1173.2 | P | 1493.3 | P |
| | | gb = U32395/gi = 1184158/ug = Mm.38795/len = 630/STRA = for | | | | | | | |
| 3 | 99073_at | Cluster Incl Z47766 Cyclin F/cds = (39,2369)/gb = Z47766/gi = 833246/ug = Mm 4008/len = 2815/STRA = for | P | 293.4 | P | 355.7 | P | 399.2 | P |
| 3 | 99448_at | Cluster Incl X56123 Mouse mRNA for talin/cds = UNKNOWN/gb = X56123/gi = 54257/ug = Mm 4053/len = 8233/STRA = for | P | 412.4 | P | 617.4 | P | 927.8 | P |
| 3 | 99471_at | Cluster Incl AF158183 AF158183 Mus musculus cDNA/gb = AF158183/gi = 5499038/ug = Mm 41271/len = 1034/STRA = for | P | 692.8 | P | 749.2 | P | 949 | P |
| 3 | 100003_at | Cluster Incl D38216 Mouse RyR1 mRNA for skeletal muscle ryanodine receptor, partial cds/cds = (0,371)/gb = D38216/ | P | 228.4 | P | 339.7 | P | 495.6 | P |
| | | gi = 1030707/ug = Mm 4519/len = 486/STRA = for | | | | | | | |
| 3 | 100436_at | Cluster Incl M27008 Orosomucoid 1/cds = (32,655)/gb = M27008/gi = 531206/ug = Mm 4777/len = 768/STRA = for | P | 329.4 | P | 227.9 | P | 695.6 | P |
| 3 | 100440_f_at | Cluster Incl U76758 Ankyrin 1, erythroid/cds = (0,164)/gb = U76758/gi = 1698953/ug = Mm 4789/len = 191/STRA = for | P | 580.1 | P | 693.2 | P | 1026.8 | P |
| 3 | 100457_at | Cluster Incl X84037: M musculus mRNA for E-selectin ligand-1/cds = (6,3533)/gb = X84037/gi = 673435/ug = Mm 488/ | P | 896.6 | P | 1044.6 | P | 1229.8 | P |
| | | len = 3887/STRA = for | | | | | | | |
| 3 | 100486_at | Cluster Incl U60453 Mus musculus enhancer of zeste homolog 1 (Ezh1) mRNA, complete cds/cds = (108,2351)/ | P | 400.8 | P | 451.8 | P | 506.1 | P |
| | | gb = U60453/gi = 1575340/ug = Mm.5027/len = 3970/STRA = for | | | | | | | |
| 3 | 100509_at | Cluster Incl AW121012 UI-M-BH2.3-aod-h-06-0-UI.s1 Mus musculus cDNA, 3 end/clone = UI-M-BH2.3-aod-h-06-0-UI/ | P | 273.7 | P | 312.5 | P | 337.6 | P |
| | | clone_end = 3/gb = AW121012/gi = 6096345/ug = Mm 5181/len = 204/STRA = for | | | | | | | |
| 3 | 100893_at | Cluster Incl U27455 Serine palmitoyltransferase, long chain base subunit 2$$/cds = (27,1709)/gb = U27455/gi = 2267216/ | P | 392.1 | P | 510.8 | P | 950.1 | P |
| | | ug = Mm 565/len = 1893/STRA = rev | | | | | | | |
| 3 | 100896_at | Cluster Incl AJ006306 Mus musculus mRNA for skeletal muscle calcium channel, gamma-subunit/cds = (131,802) | P | 265.3 | P | 389.4 | P | 801.3 | P |
| | | gb = AJ006306/gi = 3157401/ug = Mm 57093/len = 803/STRA = for | | | | | | | |
| 3 | 101383_at | Cluster Incl AJ131711.Mus musculus mRNA for troponin T/cds = (1,789)/gb = AJ131711/gi = 4468629/ug = Mm 711/len = 907/ | P | 328.7 | P | 374.1 | P | 629.5 | P |
| | | STRA = for | | | | | | | |
| 3 | 101394_at | Cluster Incl AB024922: Mus musculus mRNA for gamma-sarcogiycan, complete cds/cds = (256,1131)/gb = AB024922/ | P | 146.7 | P | 243.7 | P | 296.4 | P |
| | | gi = 5771417/ug = Mm 72173/len = 1289/STRA = for | | | | | | | |
| 3 | 101451_at | Cluster Incl AF038939 Mus musculus zinc finger protein (Peg3) mRNA, complete cds/cds = (13,4726)/gb = AF038939/ | P | 850.4 | P | 2131.9 | P | 3431 | P |
| | | gi = 2791677/ug = Mm 7952/len = 5560/STRA = for | | | | | | | |
| 3 | 101458_at | Cluster Incl D30743.Mouse mRNA for wee 1 kinase/cds = (292,2232)/gb = D30743/gi = 765088/ug = Mm.8108/len = 2270/ | P | 31.1 | P | 55.5 | P | 145.7 | P |
| | | STRA = for | | | | | | | |
| 3 | 101889_s_at | Cluster Incl U53228 RAR-related orphan receptor alpha/cds = (5,1576)/gb = U53228/gi = 1289325/ug = Mm 8858/len = 1657/ | P | 665 | P | 1086.6 | P | 1481.1 | P |
| | | STRA = for | | | | | | | |
| 3 | 101892_f_at | Cluster Incl Z67963: Fused toes/cds = (213,1091)/gb = Z67963/gi = 1067144/ug = Mm 88777/len = 2173/STRA = for | P | 274.6 | P | 275 | P | 471.3 | P |
| 3 | 101897_g_at | Cluster Incl M63697 CD1d2 antigen/cds = (293,1303)/gb = M63697/gi = 192473/ug = Mm 88779/len = 1728/STRA = for | P | 395 | P | 398.8 | P | 489.2 | P |
| 3 | 101943_at | Cluster Incl AA960259 ub61a12 s1 Mus musculus cDNA, 3 end/clone = IMAGE-1382206/clone_end = 3/gb = AA960259/ | P | 509.9 | P | 483.9 | P | 586.6 | P |
| | | gi = 3126159/ug = Mm 95636/len = 398/STRA = for | | | | | | | |
| 3 | 102223_at | Cluster Incl AF013715 Periplakin/cds = (0,331)/gb = AF013715/gi = 2992536/ug = Mm 10225/len = 776/STRA = for | P | 1785.4 | P | 2023.2 | P | 2870.8 | P |
| 3 | 102224_at | Cluster Incl AF056187 Mus musculus insulin-like growth factor I receptor mRNA, complete cds/cds = (0,4121)/ | P | 553.1 | P | 1014.6 | P | 1398.9 | P |
| | | gb = AF056187/gi = 3025893/ug = Mm 10226/len = 4500/STRA = rev | | | | | | | |
| 3 | 102304_f_at | Cluster Incl L28167 Zinc finger protein 61/cds = (927,2594)/gb = L28167/gi = 758660/ug = Mm,12832/len = 2606/STRA = for | P | 169.1 | P | 177.5 | P | 254.8 | P |
| 3 | 102866_at | Cluster Incl X94441: TEA domain family member 4/cds = (0,1283)/gb = X9441/gi = 1403383/ug = Mm.14744/len = 1284/ | P | 790.4 | P | 904.6 | P | 1449.6 | P |
| | | STRA = for | | | | | | | |
| 3 | 102867_at | Cluster Incl U51743: TEA domain family member 4/cds = (0,1138)/gb = U51743/gi = 1381555/ug = Mm.14774/len = 1339/ | P | 108.2 | P | 140.2 | P | 302.5 | P |
| | | STRA = for | | | | | | | |
| 3 | 102890_at | Cluster Incl U00677: Syntrophin, acidic 1/cds = (110,1621)/gb = U00677/gi = 404930/ug = Mm.1541/len = 2109/STRA = for | P | 677.3 | P | 975.6 | P | 1275.7 | P |
| 3 | 102915_at | Cluster Incl Z48043 Coagulation factor II (thrombin) receptor-like 1/cds = (72,1271)/gb = Z48043/gi = 663020/ug = Mm.1614/ | P | 311.3 | P | 340.9 | P | 519.2 | P |
| | | len = 2713/STRA = for | | | | | | | |
| 3 | 103218_at | Cluster Incl J04761 Mouse housekeeping DXS253E (P3) and DXS254E (GdX) genes, 3 end, and complete cds/ | P | 366.5 | P | 478.1 | P | 583.6 | P |
| | | cds = (0,549)/gb = J04761/gi = 202256/ug = Mm.19931/len = 550/STRA = for | | | | | | | |
| 3 | 103466_at | Cluster Incl AA790056 vt78h04 r1 Mus musculus cDNA, 5 end/clone = IMAGE-1177303/clone_end = 5/gb = AA790058/ | P | 150.7 | P | 232.2 | P | 437.9 | P |
| | | gi = 2850176/ug = Mm.21783/len = 490/STRA = for | | | | | | | |
| 3 | 103534_at | Cluster Incl V00722 Mouse gene for beta-1-giobin/cds = (53,496)/gb = V00722/gi = 50155/ug = Mm.2329/len = 626/STRA = for | P | 3816.4 | P | 3827.1 | P | 6362.2 | P |
| 3 | 103720_at | Cluster Incl AI449034 ms60f04.x1 Mus musculus cDNA, 3 end/clone = IMAGE-615967/clone_end = 3/gb = AI449034/ | P | 316 | P | 772.4 | P | 900.5 | P |
| | | gi = 4287173/ug = Mm.24194/len = 563/STRA = rev | | | | | | | |
| 3 | 103721_at | Cluster Incl AA592182: vo15b01 r1 Mus musculus cDNA, 5 end/clone = IMAGE-1049929/clone_end = 5/gb = AA592182/ | P | 251.3 | P | 204.6 | P | 401.4 | P |
| | | gi = 2405845/ug = Mm.24198/len = 494/STRA = for | | | | | | | |
| 3 | 103770_at | Cluster Incl AA794189 vu67d02 r1 Mus musculus cDNA, 5 end/clone = IMAGE-1196451/clone_end = 5/gb = AA794189/ | P | 228.4 | P | 240.2 | P | 1026.9 | P |
| | | gi = 2857144/ug = Mm.24776/len = 500/STRA = for | | | | | | | |
| 3 | 103958_g_at | Cluster Incl X57349 M. musculus mRNA for transferrin receptor/cds = (0,2291)/gb = X57349/gi = 54914/ug = Mm 25560/ | P | 1724.3 | P | 1976.9 | P | 3137.2 | P |
| | | len = 2292/STRA = for | | | | | | | |
| 3 | 103964_at | Cluster Incl U85259 Estrogen receptor related 1/cds = (166,1428)/gb = U85259/gi = 1916860/ug = Mm 2563/len = 2203/ | P | 547.7 | P | 715.8 | P | 847.5 | P |
| | | STRA = for | | | | | | | |
| 3 | 104188_at | Cluster Incl AI853703 UI-M-BH0-ajq-c-08-0-UI s1 Mus musculus cDNA, 3 end/clone = UI-M-BH0-ajq-c-08-0-UI/ | P | 323.4 | P | 365.7 | P | 434.3 | P |
| | | clone_end = 3/gb = AI853703/gi = 5497574/ug = Mm 27449/len = 485/STRA = for | | | | | | | |
| 3 | 104196_at | Cluster Incl AA711908 vu59e03 r1 Mus musculus cDNA, 5 end/clone = IMAGE-1195708/clone_end = 5/gb = AA711908/ | P | 246.9 | P | 292.1 | P | 325.6 | P |
| | | gi = 2721826/ug = Mm 27512/len = 474/STRA = for | | | | | | | |
| 3 | 104199_at | Cluster Incl AI646237 vv88b02.x1 Mus musculus cDNA, 3 end/clone = IMAGE-1229451/clone_end = 3/gb = AI646237/ | P | 531.6 | P | 530.9 | P | 715.1 | P |
| | | gi = 4724712/ug = Mm.27521/len = 347/STRA = for | | | | | | | |
| 3 | 104218_s_at | Cluster Incl AI507524 vl53f03 x1 Mus musculus cDNA, 3 end/clone = IMAGE-975965/clone_end = 3/gb = AI507524/ | P | 76.1 | P | 214.1 | P | 346.5 | P |
| | | gi = 4405375/ug = Mm.27913/len = 433/STRA = for | | | | | | | |
| 3 | 104250_at | Cluster Incl AW121353: UI-M-BH2 2-aom-c-12-0-UI s1 Mus musculus cDNA, 3 end/clone = UI-M-BH2.2-aom-c-12-0-UI/ | P | 361.1 | P | 398.8 | P | 573.3 | P |
| | | clone_end = 3/gb = AW121353/gi = 6096686/ug = Mm.28355/len = 562/STRA = for | | | | | | | |
| 3 | 104264_at | Cluster Incl AW122230.UI-M-BH2 2-aov-e-05-0-UI.s1 Mus musculus cDNA, 3 end/clone = UI-M-BH2.2aov-e-05-0-UI/ | P | 399.7 | P | 456.1 | P | 538.9 | P |
| | | clone_end = 3/gb = AW122230/gi = 6097693/ug = Mm.28458/len = 266/STRA = for | | | | | | | |
| 3 | 104436_at | Cluster Incl AJ250429 Mus musculus mRNA for IL-1L1 protein/cds = (112,579)/gb = AJ250429/gi = 6165412/ug = Mm.29261/ | P | 1085 | P | 799.2 | P | 1601.2 | P |
| | | len = 1284/STRA = rev | | | | | | | |
| 3 | 104453_at | Cluster Incl AW046336.UI-M-BH1-ale-f-04-0-UI s1 Mus musculus cDNA, 3 end/clone = UI-M-BH1-ale-f-04-0-UI/ | P | 573.5 | P | 646.1 | P | 823 | P |
| | | clone_end = 3/gb = AW046336/gi = 5906865/ug = Mm.2947/len = 298/STRA = rev | | | | | | | |
| 3 | 104471_at | Cluster Incl AF006603 Mus musculus histone deacetylase mHDA2 mRNA, complete cds/cds = (0,3287)/gb = AF006603/ | P | 438.5 | P | 461.1 | P | 630.5 | P |
| | | gi = 4165860/ug = Mm.29854/len = 3651/STRA = for | | | | | | | |
| 3 | 104502_f_at | Cluster Incl AI414025 ma02g05.x1 Mus musculus cDNA, 3 end/clone = IMAGE-303416/clone_end = 3/gb = AI414025/ | P | 205.3 | P | 149.4 | P | 272.1 | P |
| | | gi = 4257529/ug = Mm.30608/len = 346/STRA = for | | | | | | | |
| 3 | 104638_at | Cluster Incl U31510 ADP-ribosyltransferase 1/cds = (279,1256)/gb = U31510/gi = 1209033/ug = Mm.31099/len = 1640/ | P | 98.6 | P | 229.3 | P | 732.8 | P |
| | | STRA = for | | | | | | | |
| 3 | 104673_at | Cluster Incl X65138 Eph receptor A4/cds = (57,3017)/gb = X65138/gi = 54083/ug = Mm.3249/len = 4242/STRA = for | P | 171.7 | P | 283.5 | P | 355.3 | P |
| 3 | 104697_at | Cluster Incl AW121127.UI-M-BH2.3-aoh-d-04-0-UI s1 Mus musculus cDNA, 3 end/clone = UI-M-BH2.3-aoh-d-04-0-UI/ | P | 125 | P | 203.3 | P | 436.5 | P |
| | | clone_end = 3/gb = AW121127/gi = 6096460/ug = Mm.33722/len = 402 = STRA = for | | | | | | | |
| 3 | 92259_at | Cluster Incl AI467526: ve34d12.x1 Mus musculus cDNA, 3 end/clone = IMAGE-820055/clone_end = 3/gb = AI467526/ | P | 819.4 | P | 1071.5 | P | 1282.5 | P |
| | | gi = 4320863/ug = Mm.35645/len = 483/STRA = rev | | | | | | | |
| 3 | 92265_f_at | Cluster Incl AF042139 Mus musculus 60 kDa ribonucleoprotein Ro gene, partial cds/cds = (0,1375)/gb = AF042139/ | P | 581.1 | P | 924.7 | P | 1228.7 | P |
| | | gi = 3098483/ug = Mm 35793/len = 1376/STRA = for | | | | | | | |
| 3 | 92371_at | Cluster Incl AI505355: vq02d11.x1 Mus musculus cDNA, 3 end/clone = IMAGE-1093077/clone_end = 3/gb = AI505355/ | P | 284.6 | P | 518 | P | 899.8 | P |
| | | gi = 4403206/ug = Mm.39968/len = 421/STRA = for | | | | | | | |
| 3 | 92407_at | Cluster Incl AJ012072.Mus musculus mRNA for myomesin/skelemin/cds = (158,5158)/gb = AJ012072/gi = 5419921/ | P | 105 | P | 467.2 | P | 748.8 | P |
| | | ug = Mm.4103/len = 5645/STRA = for | | | | | | | |
| 3 | 92466_at | Cluster Incl U85714 Mus musculus phospholipase C-beta-1b mRNA, complete cds/cds = (3,3524)/gb = U85714/ | P | 784 | P | 997.9 | P | 1267.4 | P |
| | | gi = 4099296/ug = Mm.42083/len = 3853/STRA = for | | | | | | | |
| 3 | 92933_at | Cluster Incl Z18537 POU domain, class 2, transcription factor 3/cds = (75,1286)/gb = Z18537/gi = 53514/ug = Mm.4904/ | P | 669.2 | P | 846.9 | P | 1201.2 | P |
| | | len = 1267/STRA = for | | | | | | | |
| 3 | 92958_at | Cluster Incl AI849135: UI-M-AJ1-agy-b-09-0-UI.s1 Mus musculus cDNA, 3 end/clone = UI-M-AJ1-agy-b-09-0-UI/ | P | 463.1 | P | 584.4 | P | 1002.3 | P |
| | | clone_end = 3/gb = AI849135/gi = 5493041/ug = Mm.4948/len = 384 = STRA = for | | | | | | | |
| 3 | 93193_at | Cluster Incl X15643.Adrenergic receptor, beta 2/cds = (0,1256)/gb = X15643/gi = 50100/ug = Mm.5598/len = 1257/STRA = for | P | 248.5 | P | 206.8 | P | 411.5 | P |
| 3 | 93235_at | Cluster Incl AI020029 ub24c02 r1 Mus musculus cDNA, 5 end/clone = IMAGE-1378658/clone_end = 5/gb = AI020029/ | P | 287 | P | 287.7 | P | 507 | P |
| | | gi = 3234365/ug = Mm.5833/len = 468/STRA = for | | | | | | | |
| 3 | 93383_at | Cluster Incl U32107 Procollagen, type VII, alpha 1/cds = (0,8834)/gb = U32107/gi = 2326167/ug = Mm.6200/len = 9235/ | P | 1071.9 | P | 937 | P | 1546.1 | P |
| | | STRA = for | | | | | | | |
| 3 | 93451_at | Cluster Incl AW047919 UI-M-BH1-aln-c-05-0-UI.s1 Mus musculus cDNA, 3 end/clone = UI = M-BH1-aln-c-05-0-UI/ | P | 1030.6 | P | 997.6 | P | 1921.2 | P |
| | | clone_end = 3/gb = AW047919/gi = 5908448/ug = Mm.6772/len = 413/STRA = for | | | | | | | |
| 3 | 93632_g_at | Cluster Incl X95761: M. musculus mRNA for new-Rhobin/cds = UNKNOWN/gb = X95761/gi = 1771307/ug = Mm.7634/ | P | 240.4 | P | 319.6 | P | 665.9 | P |
| | | len = 2031/STRA = rev | | | | | | | |
| 3 | 93681_at | Cluster Incl AW123618: UI-M-BH2.1-api-d-09-0-UI.s1 Mus musculus cDNA, 3 end/clone = UI-M-BH2.1-api-d-09-0-UI/ | P | 383.8 | P | 716.4 | P | 887.2 | P |
| | | clone_end = 3/gb = AW123618/gi = 6099148/ug = Mm 82649/len = 416/STRA = for | | | | | | | |
| 3 | 93903_at | Cluster Incl M84120 Mouse activin receptor (ActR IIB) mRNA, complete cds/cds = (43,1653)/gb = M84120/gi = 191668/ | P | 302.9 | P | 474.8 | P | 826.3 | P |
| | | ug = Mm.8940/len = 1708/STRA = for | | | | | | | |
| 3 | 93907_f_at | Cluster Incl M17551 Mus musculus clone MIA14 full-length intracisternal A-particle gag protein gene, complete cds, and pol | P | 3264 | P | 3894.1 | P | 4763 | P |
| | | pseudogene, complete sequence/cds = (0,2483)/gb = M17551/gi = 193325/ug = Mm 89480/len = 2484/STRA = for | | | | | | | |
| 3 | 93908_f_at | Cluster Incl X16670 Mouse RNA for type IIB intracisternal A-particle (IAP) element encoding integrase, clone 106/ | P | 630.1 | P | 596.1 | P | 1407.9 | P |
| | | cds = (498,1883)/gb = X16670/gi = 51488/ug = Mm 89480/len = 1931/STRA = for | | | | | | | |
| 3 | 93909_f_at | Cluster Incl X04120 Intracisternal A particles,Thbd linked/cds = (0,2444)/gb = X04120/gi = 51502/ug = Mm 89480/len = 2445/ | P | 1050.9 | P | 852.4 | P | 2513.6 | P |
| | | STRA = for | | | | | | | |
| 3 | 94106_at | Cluster Incl AI853048 UI-M-BH0-ajn-f-11-0-UI s1 Mus musculus cDNA, 3 end/clone = UI-M-BH0-ajn-f-11-0-UI/ | P | 470.4 | P | 482.9 | P | 770.2 | P |
| | | clone_end = 3/gb = AI853048/gi = 5496954/ug = Mm 1024/len = 348/STRA = for | | | | | | | |
| 3 | 94802_at | Cluster Incl AI846678 UI-M-AN1-afi-d-11-0-UI.s1 Mus musculus cDNA, 3 end/clone = UI-M-AN1-afi-d-11-0-UI/ | P | 161.7 | P | 154.1 | P | 282.2 | P |
| | | clone_end = 3/gb = AI846678/gi = 5490584/ug = Mm 1835/len = 142/STRA = for | | | | | | | |
| 3 | 95322_g_at | Cluster Incl Y09588 Mus musculus mRNA for serotonin 4 receptor (m5-HT4(e)), splice vanant/cds = (2,1117)/gb = Y09588/ | P | 597.8 | P | 524.9 | P | 1153.5 | P |
| | | gi = 3647302/ug = Mm 20440/len = 1266/STRA = for | | | | | | | |
| 3 | 95366_at | Cluster Incl L35599 Mus musculus Y-box binding protein mRNA, 3 end/cds = (0,825)/gb = L35599/gi = 532212/ | P | 175.8 | P | 244.7 | P | 308.3 | P |
| | | ug = Mm.22482/len = 2437/STRA = for | | | | | | | |
| 3 | 96578_r_at | Cluster Incl AI853019 UI-M-BHo-ajn-d-04-0-UI s1 Mus musculus cDNA, 3 end/clone = UI-M-BH0-ajn-d-04-0-UI/ | P | 471.8 | P | 447.7 | P | 504.3 | P |
| | | clone_end = 3/gb = AI853019/gi = 5496925/ug = Mm 3021/len = 250/STRA for | | | | | | | |
| 3 | 97116_at | Cluster Incl AW123210 UI-M-BH2.1-apg-d-05-0-UI s1 Mus musculus cDNA, 3 end/clone = UI-M-BH2 1-apg-d-05-0-UI/ | P | 208.2 | P | 339.6 | P | 447.6 | P |
| | | clone_end = 3/gb = AW123210/gi = 6098780/ug = Mm 3369/len = 468/STRA for | | | | | | | |
| 3 | 97138_at | Cluster Incl C77335 C773355 Mus musculus cDNA, 3 end/clone = J0029D01/clone_end = 3/gb = C77335/gi = 2517665/ | P | 110 | P | 270.1 | P | 410.3 | P |
| | | ug = Mm 34933/len = 582/STRA = rev | | | | | | | |
| 3 | 97160_at | Cluster Incl X04017: Mouse mRNA for cysteine-rich glycoprotein SPARC/cds = (89,997)/gb = X04017/gi = 54168/ | P | 2996.6 | P | 3439.7 | P | 4374.5 | P |
| | | ug = Mm 35439/len = 2079/STRA = for | | | | | | | |
| 3 | 97181_f_at | Cluster Incl M10062 Mouse IgE-binding factor mRNA, complete cds/cds = UNKNOWN/gb = M10062/gi = 192166/ | P | 2980.4 | P | 4163.4 | P | 4916.9 | P |
| | | ug = Mm 35835/len = 3305/STRA = for | | | | | | | |
| 3 | 97759_at | Cluster Incl U09383 Mus musculus calcium-activated potassium channel mRNA, complete cds/cds = (820,4374)/ | P | 131.5 | P | 188.5 | P | 239.5 | P |
| | | gb = U09383/gi = 487796/ug = Mm 4123/len = 5001/STRA = for | | | | | | | |
| 3 | 98278_at | Cluster Incl AB019280 Mus musculus mRNA for sprouty-4, complete cds/cds = (215,1117)/gb = AB019280/gi = 4850325/ | P | 554.9 | P | 737.4 | P | 971.7 | P |
| | | ug = Mm 42038/len = 1131/STRA = for | | | | | | | |
| 3 | 99405_at | Cluster Incl U30241 Mus musculus anti-DNA antibody Ig kappa chain mRNA, v-j region, hybndoma 84.15, partial cds/ | P | 116.3 | P | 232.9 | P | 284.9 | P |
| | | cds = (0,298)/gb = U30241/gi = 896180/ug = Mm 57033/len = 299/STRA for | | | | | | | |
| 3 | 99823_r_at | Cluster Incl C79676 C79676 Mus musculus cDNA, 3 end/clone = J0070C12/clone_end = 3/gb = C79676/gi = 2520006/ | P | 314.5 | P | 437.9 | P | 478.6 | P |
| | | ug = Mm 5866/len = 532/STRA = rev | | | | | | | |
| 3 | 99875_at | Cluster Incl Z32675 Hairless/cds = (377,3925)/gb = Z32675/gi 531706/ug = Mm 7598/len = 4017/STRA = for | P | 371.2 | P | 514.7 | P | 816 | P |
| 3 | 99896_at | Cluster Incl U08354 Melanocortin 5 receptor/cds = (28,1146)/gb = U08354/gi = 522165/ug = Mm 8003/len 1180/STRA = for | P | 233.9 | P | 522.8 | P | 620.7 | P |
| 3 | 100293_at | Cluster Incl AA268823 vb01d09 r1 Mus musculus cDNA, 5 end/clone = IMAGE-747665/clone_end = 5/gb = AA268823/ | P | 223.7 | P | 267.1 | P | 348.1 | P |
| | | gi = 1905739/ug = Mm 86378/len = 435/STRA = for | | | | | | | |
| 3 | 96963_s_at | Cluster Incl L14553 Mus musculus Ig light chain V-region CC49 rearranged gene, exons 1 and 2/cds = (0,399)/gb = L14553/ | P | 119.1 | P | 132.3 | P | 289.5 | P |
| | | gi = 293639/ug = Mm 88797/len = 400/STRA = for | | | | | | | |
| 3 | 97058_f_at | Cluster Incl AW208630: uo62a10.x1 Mus musculus cDNA, 3 end/clone = IMAGE-2647098/clone_end = 3/gb = AW208630/ | P | 493.4 | P | 424 | P | 831.3 | P |
| | | gi = 6514570/ug = Mm 89273/len = 197/STRA = rev | | | | | | | |
| 3 | 97659_f_at | Cluster Incl X04725.Insulin I/cds = (0,326)/gb = X04725/gi = 52712/ug = Mm 89940/len = 327/STRA = for | P | 784.4 | P | 934.9 | P | 1228.4 | P |
| 3 | 98245_at | Cluster Incl C77789 C77789 Mus musculus cDNA, 3 end/clone = J0037F03/clone_end = 3/gb = C77789/gi = 2518119/ | P | 209.1 | P | 330.4 | P | 664.1 | P |
| | | ug = Mm 91361/len = 623/STRA = rev | | | | | | | |
| 3 | 100225_f_at | Cluster Incl AA09481 EST01210 Mus musculus cDNA, 3 end/clone = C0007B09/clone_end = 3/gb = AA409481/ | P | 287.5 | P | 273.1 | P | 443.6 | P |
| | | gi = 2066125/ug = Mm.98618/len = 254/STRA = rev | | | | | | | |
| 3 | 101152_at | Cluster Incl Z18278 5-hydroxytryptamine (serotonin) receptor 5A/cds = (502,1575)/gb = Z18278/gi = 49758/ug = Mm 4835/ | P | 337.7 | P | 498.1 | P | 559.6 | P |
| | | len = 1680/STRA = for | | | | | | | |
| 3 | 101186_at | Cluster Incl X65588 M musculus mp41 mRNA/cds = (15,416)/gb = X65588/gi = 1524150/ug = Mm 5118/len = 2857/STRA = for | P | 211.2 | P | 313 | P | 358.4 | P |
| 3 | 101217_at | cluster Incl D18885 MUSGS01047 Mus musculus cDNA, 3 end/clone = mc0128/clone_end = 3/gb = D18865/gi = 1089498/ | P | 226 | P | 212 | P | 389.2 | P |
| | | ug = Mm.5344/len = 314/STRA = rev | | | | | | | |
| 3 | 92572_at | Cluster Incl AI509617 vx14h07.y1 Mus musculus cDNA, 5 end/clone = IMAGE-1264477/clone_end = 5/gb = AI509617/ | P | 145.7 | P | 136.3 | P | 176 | P |
| | | gi = 4408522/ug = Mm 10331/len = 448/STRA = rev | | | | | | | |
| 3 | 92887_at | Cluster Incl AF004106 Mus musculus unknown mRNA, complete cds/cds = (234,968)/gb = AF004106/gi = 2183320/ | P | 1177.8 | P | 1583 | P | 1785.4 | P |
| | | ug = Mm 1457/len = 1270/STRA = for | | | | | | | |
| 3 | 93020_at | Cluster Incl AF051347 Mus musculus REX-3 mRNA, complete cds/cds = (228,578)/gb = AF051347/gi = 3510642/ | P | 63.8 | P | 104.4 | P | 175 | P |
| | | ug = Mm.14768/len = 838/STRA = for | | | | | | | |
| 3 | 93821_at | Cluster Incl AW046101.UI-M-BH1-alf-b-06-0-UI s1 Mus musculus cDNA, 3 end/clone = UI-M-BH1-alf-b-06-0-UI/ | A | 187.3 | P | 282.4 | P | 346.9 | P |
| | | clone_end = 3/gb = AW046101/gi = 5906630/ug = Mm 21514/len = 261/STRA = rev | | | | | | | |
| 3 | 94877_at | Cluster Incl AI848446 UI-M-AH1-ags-f-03-0-UI s1 Mus musculus cDNA, 3 end/clone = UI-M-AH1-ags-f-03-0-UI/ | P | 86.1 | P | 117.4 | P | 163.5 | P |
| | | clone_end = 3/gb = AI848446/gi = 5492352/ug = Mm.23839/len = 514/STRA = for | | | | | | | |
| 3 | 94472_at | Cluster Incl AW123216 UI-M-BH2.1-apg-d-12-0-UI s1 Mus musculus cDNA, 3 end/clone = UI-M-BH2 1-apg-d-12-0-UI/ | P | 193.1 | P | 136.4 | P | 392.3 | P |
| | | clone_end = 3/gb = AW123216/gi = 6098786/ug = Mm 28852/len = 287/STRA = for | | | | | | | |
| 3 | 97482_at | Cluster Incl AA832565: vw43b11.r1 Mus musculus cDNA, 5 end/clone = IMAGE-1246557/clone_end = 5/gb = AA832565/ | P | 331.1 | P | 468.7 | P | 566.1 | P |
| | | gi = 2906293/ug = Mm.30762/len = 478/STRA = for | | | | | | | |
| 3 | 97536_at | Cluster Incl AW046650: UI-M-BH1-ald-e-10-0-UI s1 Mus musculus cDNA, 3 end/clone = UI-M-BH1-aid-e-10-0-UI/ | A | 347.7 | P | 519.5 | P | 603.3 | P |
| | | clone_end = 3/gb = AW046650/gi = 5907179/ug = Mm3308/len = 336/STRA = for | | | | | | | |
| 3 | 100997_at | Cluster Incl L22550: Desmin/cds = UNKNOWN/gb = gb 32 L22550/gi = 348971/ug = Mm 6712/len = 1497/STRA = for | P | 389.9 | P | 566.4 | P | 1352.4 | P |
| 3 | 103616_at | Cluster Incl AI181132.ub95c0.r1 Mus musculus cDNA, 5 end/clone = IMAGE-1396224/clone_end = 5/gb = AI181132/ | P | 338.5 | P | 177 | P | 859.5 | P |
| | | gi = 3731770/ug = Mm20240/len = 499/STRA = for | | | | | | | |
| 3 | 104515_at | Cluster Incl AI286818 uc55b02 r1 Mus musculas cDNA, 5 end/clone = IMAGE-1429515/clone_= 5/gb = AI286818/ | P | 159.2 | P | 115.7 | P | 268.6 | P |
| | | gi = 3926571/ug = Mm.22765/len = 417/STRA = for | | | | | | | |
| 3 | 104529_at | Cluster Incl U21960: Calcium modulating ligand/cds = (34,918)/gb = U21960/gi = 726183/ug = Mm 2313/len = 1331/STRA = for | P | 149.7 | P | 174.2 | P | 204.8 | P |
| 3 | 98461_at | Cluster Incl AI987835 um07f07.x1 Mus musculus cDNA, 3 end/clone = IMAGE-2182405/clone _end = 3/gb = AI987935/ | P | 267.2 | P | 317 | P | 394.2 | P |
| | | gi = 5819629/ug = Mm 34789/len = 623/STRA = for | | | | | | | |
| 3 | 100429_at | Cluster Incl U89155 untitled/cds = (0,149)/gb = U89155/gi = 2707592/ug = Mm 4730/len = 150/STRA = for | P | 128.6 | P | 139.8 | P | 257.6 | P |
| 3 | 100935_at | Cluster Incl U43548 Mus musculus trasncription factor like protein 4 TCFL4 mRNA, partial cds/cds(0,892)/gb = U43548/ | P | 524.9 | P | 639.9 | P | 712.5 | P |
| | | gi = 1927189/ug = Mm.628/len = 1963/STRA = for | | | | | | | |
| 3 | 100962_at | Cluster Incl U47543 Ngfi-A binding protein 2/cds = (220,1797)/gb = U47543/gi = 197730/ug = Mm 6549/len = 2539 | P | 202.7 | P | 221.6 | P | 251.3 | P |
| | | STRA = for | | | | | | | |
| 3 | 101359_at | Cluster Incl U43541 Lamin,beta 2/cds = (0,5399)/gb = U43541/gi = 1244719/ug = Mm 68867/len = 5400/STRA = for | P | 164.9 | P | 189.2 | P | 302 | P |
| 3 | 101861_at | Cluster lncl AF031919 Sarcoglycan, epsilon/cds = (44,1285)/gb = AF031919/gi = 3057139/ug = Mm.8739/len = 1548 | P | 388.6 | P | 359.9 | P | 578.5 | P |
| 3 | 101913_at | Cluster Incl AW214298 uo39g06.x1 Mus musculus cDNA, 3 end/clone = IMAGE-2644954/clone_end = 3/gb = AW214298/ | P | 234.6 | P | 204.3 | P | 354 | P |
| | | gi = 6520787/ug = Mm.90654/len = 172/STRA = for | | | | | | | |
| 3 | 102878_at | Cluster Incl U12135 RAD52 homolog, (S cerevisiae/cds = (114,1373)/gb = U12135/gi = 602158/ug = Mm 149/len = 1664/ | P | 354.2 | P | 355.2 | P | 447.8 | P |
| | | STRA = for | | | | | | | |
| 3 | 103547_at | Cluster Incl AI837116 UI-M-AK0-adc-e-09-0-UI a1 Mus musculus cDNA, 3 end/clone = UI-M-AK0-adc-e-09-0-UI/ | P | 246.5 | P | 320.4 | P | 401.3 | M |
| | | clone_end = 3/gb = AI837116/gi = 5471329/ug = Mm 23723/len = 323/STRA = rev | | | | | | | |
| 3 | 103927_at | Cluster Incl AW212672 uo10c08 x1 Mus musculus cDNA, 3 end/clone = IMAGE-2599118/clone_end = 3/gb = AW212672/ | P | 163.5 | P | 118 | P | 400.3 | P |
| | | gi = 6518785/ug = Mm 25086/len = 154/STRA = for | | | | | | | |
| 3 | 104164_at | Cluster Incl AI181257 ub94b03 r1 Mus musculus cDNA, 5 end/clone = IMAGE-1396109/clone_end = 5/gb = AI181257/ | P | 96.2 | P | 144 | P | 331.1 | P |
| | | gi = 3731895/ug = Mm.27139/len = 510/STRA = for | | | | | | | |
| 3 | 104252_at | Cluster Incl AW123823 UI-M-BH2 1-apm-g-07-0-UI s1 Mus musculus cDNA, 3 end/clone = UI-M-BH2 1-apm-g-07-0-UI/ | P | 147.1 | P | 191.9 | P | 297.1 | P |
| | | clone_end = 3/gb = AW123823/gi = 6099353/ug = Mm 28362/len = 386/STRA = for | | | | | | | |
| 3 | 104265_at | Cluster Incl X70842 Kinase insert domain protein receptor/cds = (285,4388)/gb = X70842/gi = 57923/ug = Mm.285/len = 5470/ | P | 375.6 | P | 340.1 | P | 673.8 | P |
| | | STRA = for | | | | | | | |
| 3 | 92269_r_at | Cluster Incl AI836610 UI-M-AP0-abf-g-08-0-UI.s1 Mus musculus cDNA, 3 end/clone = UI-M-AP0-abf-g-08-0-UI/ | P | 96 | P | 77.4 | A | 168.6 | P |
| | | clone_end = 3/gb = AI836610/gi = 5470823/ug = Mm.3590/len = 130/STRA = for | | | | | | | |
| 3 | 92518_at | Cluster Incl Y09535 M musculus mRNA for neogenin protein/cds = (180,4661)/gb = Y09535/gi = 1881476/ug = Mm 42249/ | P | 402.3 | P | 390.3 | A | 708.2 | P |
| | | len = 5414/STRA = for | | | | | | | |
| 3 | 92691_at | Cluster Incl AW048155 UI-M-BH1-alq-h-08-0-UI s1 Mus musculus cDNA, 3 end/clone = UI-M-BH1-alq-h-08-0-UI/ | P | 467.3 | P | 771.7 | P | 1002.1 | P |
| | | clone_end = 3/gb = AW048155/gi = 5908684/ug = Mm 45608/len = 425/STRA = for | | | | | | | |
| 3 | 92942_at | Cluster Incl AA138192: mr05c12 r1 Mus musculus cDNA, 5 end/clone = IMAGE-596566/clone_end = 5/gb = AA138192/ | P | 452.7 | P | 439.3 | P | 655.2 | P |
| | | gi = 1700519/ug = Mm 492/len = 468/STRA = rev | | | | | | | |
| 3 | 93191_at | Cluster Incl AW061328 UI-M-BH1-anw-f-01-0-UI s1 Mus musculus cDNA, 3 end/clone = UI-M-BH1-anw-f-01-0-UI/ | P | 168 | P | 236.9 | P | 333.3 | P |
| | | clone_end = 3/gb = AW061328/gi = 6009079/ug = Mm 5572/len = 461/STRA = rev | | | | | | | |
| 3 | 93392_at | Cluster Incl AB010742: Uncoupling protein 3, mitochondrial/cds = (197,1123)/gb = AB010742/gi = 3062840/ug = Mm.6254/ | P | 229.3 | P | 259.7 | P | 365 | P |
| | | len = 2460/STRA = rev | | | | | | | |
| 3 | 93690_at | Cluster Incl AF022072 Growth factor receptor bound protein 10/cds = (308,2098)/gb = AF022072/gi = 2465448/ug = Mm 8432/ | P | 321.3 | P | 404.5 | P | 662.8 | P |
| | | len = 2176/STRA = for | | | | | | | |
| 3 | 94174_at | Cluster Incl AF006071 Mus musculus alpha-catenin related protein (ACRP) mRNA, complete cds/cds = (84,2279)/ | P | 52 | A | 66.6 | P | 239.3 | P |
| | | gb = AF006071/gi = 3309106/ug = Mm.12878/len = 2292/STRA = for | | | | | | | |
| 3 | 94790_at | Cluster Incl AA681807: vu65f09 r1 Mus musculus cDNA, 5 end/clone = IMAGE-1196297/clone_end = 5/gb = AA681807/ | P | 98.9 | P | 124.1 | P | 554.8 | P |
| | | gi = 2663947/ug = Mm 17221/len = 318/STRA = rev | | | | | | | |
| 3 | 96541_at | Cluster Incl AI838636 UI-M-AO0-aca-a-10-0-UI.s1 Mus musculus cDNA, 3 end/clone = UI-M-AO0-aca-a-10-0-UI/ | P | 170.5 | P | 153.8 | P | 284.9 | A |
| | | clone_end = 3/gb = AI838636/gi = 5472849/ug = Mm 27795/len = 369/STRA = rev | | | | | | | |
| 3 | 98869_g_at | Cluster Incl L31532 B-cell leukemia/lymphoma 2/cds = (1197,1796)/gb = L31532/gi = 468337/ug = Mm.5155/len = 1897/ | P | 207.2 | M | 314.4 | P | 420 | P |
| | | STRA = for | | | | | | | |
| 3 | 99813_g_at | Cluster Incl X92523 Calpain 3/cds = (0,2465)/gb = X92523/gi = 1246778/ug = Mm 57235/len = 2466/STRA = for | M | 37.5 | P | 43.9 | P | 102.4 | P |
| 3 | 99825_at | Cluster Incl AA881307: vx11d09.r1 Mus musculus cDNA, 5 end/clone = IMAGE-1264145/clone_end = 5/gb = AA881307/ | P | 307 | P | 285.9 | P | 388.5 | P |
| | | gi = 2990617/ug = Mm 5980/len = 511/STRA = rev | | | | | | | |
| 3 | 99842_at | Cluster Incl AB000636: Procollagen, type XIX, alpha 1/cds = (98,3508)/gb = AB000636/gi = 2627208/ug = Mm 6252/len = 4205/ | P | 107.7 | P | 121.8 | P | 197.5 | P |
| | | STRA = for | | | | | | | |
| 3 | 100366_at | Cluster Incl AB019600: Mus musculus mRNA for caspase9, complete cds/cds = (0,1364)/gb = AB019600/gi = 6440941/ | P | 283.3 | P | 311.1 | A | 446.4 | P |
| | | ug = Mm.88829/len = 1365/STRA = for | | | | | | | |
| 3 | 101716_at | Cluster Incl AF017280 Mus musculus ribonuclease 5 precursor (mR-5) gene, complete cds/cds = (0,467)/gb = AF017260/ | P | 77.9 | P | 84.4 | P | 117.8 | P |
| | | gi = 2407241/ug = Mm 57025/len = 468/STRA = for | | | | | | | |
| 3 | 101830_at | Cluster Incl X83934: Ryanodine receptor 3/ods = (0,866)/gb = X83934/gi = 639816/ug = Mm 57201/len = 867/STRA = for | P | 126 | P | 188 | P | 278.8 | P |
| 3 | 101633_at | Cluster Incl X16678: Immunoglobulin kappa chain variable 20 (V20 family)/cds = (0,383)/gb = X16678/gi = 55388/ | P | 542.3 | P | 368.7 | P | 1094.4 | P |
| | | ug = Mm 6294/len = 384/STRA = for | | | | | | | |
| 3 | 99187_f_at | Cluster Incl AI835662: UI-M-AJ0-aau-e-03-0-UI s1 Mus musculus cDNA, 3 end/clone = UI-M-AJ0-aau-e-03-0-UI/ | P | 333.5 | P | 468.7 | P | 830.7 | A |
| | | clone_end = 3/gb = AI835662/gi = 5469875/ug = Mm 41890/len = 268/STRA = for | | | | | | | |
| 3 | 103578 _at | Cluster Incl AJ006972: Mus musculus mRNA for TOM1 protein/cds = (67,1545)/gb = AJ006972/gi = 3319987/ug = Mm.1967/ | P | 242.5 | P | 171 | A | 415.7 | P |
| | | len = 2253/STRA = for | | | | | | | |
| 3 | 104591_g_at | Cluster Incl 13171: Myocyte enhancer factor 2C/cds = UNKNOWN/gb = L13171/gi = 293728/ug = Mm.24001/len = 1656/ | P | 60 | A | 88.7 | P | 186.4 | P |
| | | STRA = for | | | | | | | |
| 3 | 97944_f_at | Cluster Incl AF099808: Mus musculus T cell receptor alpha chain variable region (Tcra-V11 1d) gene, allele S1, partial cds/ | P | 258.1 | P | 261.2 | P | 596.2 | P |
| | | cds = (0,233)/gb = AF099808/gi = 4323544/ug = Mm 30295/len = 234/STRA = for | | | | | | | |
| 3 | 101893_r_at | Cluster Incl z67963 Fussed toes/cds = (213,1091)/gb = Z67963/gi = 1067144/ug = Mm 88777/len = 2173/STRA = for | P | 24.8 | P | 23.9 | A | 42.7 | P |
| 3 | 103302_r_at | Cluster Incl AA866668 ud07a10 r1 Mus musculus cDNA, 5 end/clone = IMAGE-1424426/clone_end = 5/gb = AA866668/ | P | 323.2 | P | 340.4 | P | 366.1 | P |
| | | gi = 2962113/ug = Mm 21097/len = 537/STRA = for | | | | | | | |
| 3 | 92401_at | Cluster Incl U27195 Leukotnene C4 synthase/cds = (36,488)/gb = U27195/gi = 973221/ug = Mm.4088/len = 588/STRA = for | P | 168.4 | P | 186.7 | P | 241 | P |
| 3 | 92519_at | Cluster Incl X74616 Phosphorylase kinase alpha 1/cds = (218,3943)/gb = X74616/gi= 436123/ug = Mm 42254/len = 4130/ | P | 233.1 | P | 249.7 | A | 414.9 | P |
| | | STRA = for | | | | | | | |
| 3 | 94715_at | Cluster Incl K02588 Cytochrome P450, 1a1, aromatic compound inducible/cds = (109,1674)/gb = K02588/gi = 192885/ | P | 313.1 | P | 253.6 | P | 458 | P |
| | | ug = Mm 14089/len = 2619/STRA = for | | | | | | | |
| 3 | 97743_at | Cluster Incl AW047461 UI-M-BH1-all-e-12-0-UI s1 Mus musculus cDNA, 3 end/clone = UI-M-BH1-all-e-12-0-UI/ | A | 1215.7 | P | 1496.6 | P | 1985.3 | P |
| | | clone_end = 3/gb = AW047461/gi = 5907990/ug = Mm 4016/len = 254/STR = rev | | | | | | | |
| 3 | 98847₁₃ at | Cluster Incl U52826 Mus musculus syndecan-3 mRNA, complete cds/cds = (34,1362)/gb = U52826/gi = 1399582/ | P | 319.4 | P | 365.1 | A | 507.9 | P |
| | | ug = Mm.5059/len = 1495/STRA = for | | | | | | | |
| 3 | 97002_f_at | Cluster Incl AJ133427 Mus musculus or37d pseudogene/cds = (0,962)/gb = AJ133427/gi = 5869921/ug = Mm 88844/len = 963/ | P | 237.6 | P | 349.6 | A | 514.3 | A |
| | | STRA = for | | | | | | | |
| 3 | 102113_f_at | Cluster Incl AI324061 mt75g06 x1 Mus musculus cDNA, 3 end/clone = IMAGE-635770/clone_end = 3/gb = AI324061/ | P | 87.6 | P | 275.8 | P | 473.7 | P |
| | | gi = 4058490/ug = Mm 42606/len = 258/STRa = rev | | | | | | | |
| 3 | 92786_at | Cluster Incl AI626942.vs77b11.x1 Mus musculus cDNA, 3 end/clone = IMAGE-1152285/clone_end= 3/gb= AI626942/ | P | 113.4 | P | 159.2 | P | 214.4 | P |
| | | gi = 4663742/ug = Mm 12454/len = 306/STRA = for | | | | | | | |
| 3 | 93359_at | Cluster Incl AI839743 UI-M-AP0-abe-e-06-0-UI s2 Mus musculus cDNA, 3 end/clone = UI-m-APO-abe-e-06-0-UI/ | P | 116.2 | P | 153.1 | A | 171.9 | A |
| | | clone_end = 3/gb = AI839743/gi = 5473956/ug = Mm 1893/len = 456/STRA = for | | | | | | | |
| 3 | 93592_at | Cluster Incl X82648 M musculus mRNA for apolipoprotein D/cds = (89,658)/gb = X82648/gi = 575656/ug = Mm 2082/len = 903/ | P | 145.1 | P | 93.1 | A | 270.8 | P |
| | | STRA = for | | | | | | | |
| 3 | 94857_at | Cluster Incl U10420 N-methylpurine-DNA glycosylase/cds = (29,1030)/gb = U10420/gi = 500754/ug = Mm 2358/len = 1061/ | P | 102.4 | P | 121 | A | 203 | A |
| | | STRA = for | | | | | | | |
| 3 | 102070_at | Cluster Incl AW212495 uo89g01 x1 Mus musculus cDNA, 3 end/clone = IMAGE-2649744/clone_end = 3/gb = AW212495/ | A | 597.4 | P | 788.1 | P | 892.2 | P |
| | | gi = 6518582/ug = Mm.106/len = 429/STRA = for | | | | | | | |
| 3 | 102312_r_at | Cluster Incl AW046728 UI-M-BH1-alt-g-05-0-UI s1 Mus musculus cDNA, 3 end/clone = UI-M-BH1-alt-g-05-0-UI/ | P | 594.6 | P | 850.7 | P | 1333.3 | A |
| | | clone_end = 3/gb = AW046728/gi = 5907297/ug = Mm.10641/len = 302/STRA = for | | | | | | | |
| 3 | 102314_at | Cluster Incl M23383 Solute carrier family 2 (facilitated glucose transporter), member 4/cds = (153,1685)/gb = M23383/ | P | 141.9 | P | 159.4 | A | 393.1 | P |
| | | gi = 193706/ug = Mm 10661/len = 2521/STRA = for | | | | | | | |
| 3 | 103369_at | Cluster Incl AW049142 UI-M-BH1-amq-b-11-0-UI a1 Mus musculus cDNA, 3 end/clone = UI-M-BH1-amq-b-11-0-UI/ | P | 402.2 | P | 515.1 | P | 651.5 | P |
| | | clone_end = 3/gb = AW049142/gi = 5909671/ug = Mm.18538/len = 446/STRA = rev | | | | | | | |
| 3 | 103377_at | Cluster Incl AW259788 um81b12 y1 Mus musculus cDNA, 5 end/clone = IMAGE-2317727/clone_end = 5/gb = AW259788/ | P | 81.3 | A | 179 | P | 193.5 | P |
| | | gi = 6632769/ug = Mm.18600/len = 507/STRA = for | | | | | | | |
| 3 | 103838_at | Cluster Incl AB010144 Mus musculus gene for mitsugumin29/cds = (183,977)/gb = AB010144/gi = 3761106/ug = Mm 20942/ | P | 321.3 | A | 480.8 | P | 604.3 | P |
| | | len = 3248/STRA = for | | | | | | | |
| 3 | 94977_at | Cluster Incl X15373 Inositol 1,4,5-triphosphate receptor 1/cds = (328,8577)/gb = X15373/gi = 53568/ug = Mm 2726/len = 9841/ | P | 43.1 | P | 20.6 | P | 73.8 | P |
| | | STRA = for | | | | | | | |
| 3 | 97403_at | Cluster Incl AW045743 UI-M-BH1-akq-h-05-0-UI s1 Mus musculus cDNA, 3 end/clone = UI-M-BH1-akq-h-05-0-UI/ | P | 242.6 | P | 225.5 | A | 383.3 | A |
| | | clone_end = 3/gb = AW045743/gi = 5906272/ug = Mm 29900/len = 257/STRA = for | | | | | | | |
| 3 | 98513_at | Cluster Incl AI851821: UI-M-BH0-ais-d-03-0-UI s1 Mus musculus cDNA, 3 end/clone = UI-M-BH0-ais-d-03-0-UI/ | P | 371.6 | P | 328.4 | P | 816.6 | M |
| | | clone_end = 3/gb = AI851821/gi = 5495727/ug = Mm 3600/len = 420/STRA = for | | | | | | | |
| 3 | 99050_at | Cluster Incl U36579 Caveolin 3/cds = (121,576)/gb = U36579/gi = 1155228/ug = Mm 3924/len = 1000/STRA = for | P | 321.4 | P | 238.4 | A | 1097.8 | P |
| 3 | 102639_at | Cluster Incl AB011451 Mus musculus mRNA for N-acetylgucosamine-6-O-sulfotransferase, complete cds/cds = (469,1920)/ | A | 100 | P | 102 | P | 128 | P |
| | | gb = AB011451/gi = 3402294/ug = Mm.12968/len = 2150/STRA = for | | | | | | | |
| 3 | 103212_at | Cluster Incl AI314784 uj35c08 x1 Mus musculus cDNA, 3 end/clone = IMAGE-1921934/clone_end = 3/gb = AI314784/ | A | 167.6 | P | 256 | P | 450.2 | P |
| | | gi = 4030051/ug = Mm 19726/len = 494/STRA = for | | | | | | | |
| 3 | 104678_at | Cluster Incl U19860 Growth arrest specific 7/cds = (130,1395)/gb = U19860/gi = 1154621/ug = Mm.3257/len = 2888/ | P | 120.5 | P | 150.9 | A | 188.9 | P |
| | | STRA = for | | | | | | | |
| 3 | 92323_at | Cluster Incl Y13439 Mus musculus mRNA for stress activated protein kinase-3/cds = (167,1270)/gb = Y13439/gi = 2181949/ | P | 159.5 | P | 201.2 | P | 435.2 | P |
| | | ug = Mm 38343/len = 1553/STRA = for | | | | | | | |
| 3 | 92387_at | Cluster Incl L18868 Thromboxane A synthase 1, platelet/cds = (38,1839)/gb = L18868/gi = 1763715/ug = Mm.4054/len = 1847/ | A | 871.9 | P | 882.1 | P | 1037.6 | P |
| | | STRA = rev | | | | | | | |
| 3 | 92535_at | Cluster Incl L12147 Earty B-cell factor/cds = (45,1820)/gb = L12147/gi = 309208/ug = Mm 4366/len = 2248/STRA = for | P | 169.3 | P | 118.6 | A | 299.2 | P |
| 3 | 93893_f_at | Cluster Incl U56404: Mus musculus strain C57BL/8 natural killer cell receptor Ly-49C mRNA, complete cds/cds = (49,849)/ | P | 101.8 | P | 112.6 | P | 157 | P |
| | | gb = U56404/gi = 1336030/ug = Mm 89187/len = 907/STRA = for | | | | | | | |
| 3 | 94757_at | Cluster Incl M60493 Cystic fibrosis transmembrane conductance regulator homolog/cds = (138,4568)/gb = M60493/ | A | 65.3 | M | 93.5 | P | 137.7 | P |
| | | gi = 192831/ug = Mm.15621/len = 6304/STRA = for | | | | | | | |
| 3 | 95358_at | Cluster Incl AI843864 UI-M-AK1-aeu-c-11-0-UI s1 Mus musculus cDNA, 3 end/clone = UI-M-AK1-aeu-c-11-0-UI/ | P | 284.1 | P | 203.9 | A | 495 | A |
| | | clone_end = 3/gb = AI843864/gi = 5478077/ug = Mm 21518/len = 341/STRA = for | | | | | | | |
| 3 | 97158_at | Cluster Incl AI840785 UI-M-AM0-adn-h-05-0-UI.s1 Mus musculus cDNA, 3 end/clone = UI-M-AM0-adn-h-05-0-UI/ | P | 229.1 | P | 316 | A | 336.8 | P |
| | | clone_end = 3/gb = AI840785/gi = 5474998/ug = Mm.35235/len = 324/STRA = rev | | | | | | | |
| 3 | 97761_f_at | Cluster Incl U10094 Killer cell lectin-like receptor, subfamily A, member 7/cds = (70,873)/gb = U10094/gi = 533495/ | A | 221.6 | P | 319.1 | A | 404.1 | P |
| | | ug = Mm 4131/len = 1075/STRA = for | | | | | | | |
| 3 | 98782_at | Cluster Incl D38613: Mouse 921-L mRNA for presynaptic protein, complete cds/cds = (84,488)/gb = D38613/gi = 1041029/ | A | 324.1 | P | 432.3 | P | 493.8 | P |
| | | ug = Mm.4705/len = 1080/STRA = for | | | | | | | |
| 3 | 99435_at | Cluster Incl Z22703 Fibroblast growth factor 7/cds = (0,584)/gb = Z22703/gi = 297755/ug = Mm 57177/len = 585/STRA = for | P | 129.1 | P | 306 | P | 324.9 | P |
| 3 | 101684_r_at | Cluster Incl X67863 M musculus T2 mRNA/cds = (174,731)/gb = X67863/gi = 296381/ug = Mm 35868/len = 1263/STRA = for | P | 590.4 | P | 835.6 | P | 1038.3 | A |
| 3 | 93294_at | Cluster Incl M70642: Fibroblast Inducible secreted protein/cds = (137,1183)/gb = M70642/gi = 193315/ug = Mm.1810/ | A | 86.1 | P | 179.3 | P | 256.8 | P |
| | | len = 2267/STRA = for | | | | | | | |
| 3 | 93532_at | Cluster Incl J04992 Troponin I, skeletal, fast 2/cds = (23,571)/gb = J04992/gi = 202164/ug = Mm 1984/len = 667/STRA = for | P | 373.2 | A | 560.8 | P | 1659.5 | P |
| 3 | 95401_at | Cluster Incl AW050156: UI-M-BH1-ano-g-04-0-UI s1 Mus musculus cDNA, 3 end/clone = UI-M-BH1-ano-g-04-0-UI/ | P | 625.1 | P | 719.5 | P | 1016.3 | A |
| | | clone_end = 3/gb = AW050156/gi = 5910685/ug = Mm.24195/len = 443/STRA = for | | | | | | | |
| 3 | 98106_at | Cluster Incl U69898 Mus musculus translocase of inner mitochondrial membrane (Tim44) mRNA, nuclear gene encoding | P | 666.7 | P | 587.6 | A | 790.5 | P |
| | | mitochondrial protein, complete cds/cds = (8,1366)/gb = U69898/gi = 2351409/ug = Mm.34791/len = 1764/STRA = for | | | | | | | |
| 3 | 103815_at | Cluster Incl AI852311 UI-M-BH0-aje-g-04-0-UI s1 Mus musculus cDNA, 3 end/clone = UI-M-BH0-aje-g-04-0-UI/ | P | 193.8 | P | 196.3 | P | 229.7 | A |
| | | clone_end = 3/gb = AI852311/gi = 5496217/ug = Mm.20901/len = 471/STRA = for | | | | | | | |
| 3 | 94442_s_at | Cluster Incl AF030001: Mus musculus major histocompatibility locus class III region-butyrophilin-like protein gene, partial | A | 278.8 | P | 312.6 | A | 420.9 | P |
| | | cds, Notch4, PBX2, RAGE, lysophatidic acid acyl transferase-alpha, palmitoyl-protein thioesterase 2 (PPT2), CREB-RP, | | | | | | | |
| | | and tenascin X/STRA = for | | | | | | | |
| 3 | 97336_at | Cluster Incl AJ131851: Mus musculus mRNA for cathepsin F/cds = (16,1404)/gb = AJ131851/gi = 4826564/ug = Mm.29561 | P | 179.4 | M | 214.5 | M | 371.1 | P |
| | | len = 1784/STRA = for | | | | | | | |
| 3 | 97379_at | Cluster Incl D42083: Mouse mRNA for fructose 1,6-bisphosphatase, complete cds/cds = (19,1083)/gb = D42083/gi = 575935/ | P | 88.2 | M | 185.4 | P | 416.4 | P |
| | | ug = Mm 2974/len = 1242/STRA = for | | | | | | | |
| 3 | 98498_at | Cluster Incl D86353: Mouse mRNA for Mch3, complete cds/cds = (52,963)/gb = D86353/gi = 1945545/ug = Mm 35687/ | P | 101.5 | P | 34.9 | A | 268 | A |
| | | len = 964/STRA = for | | | | | | | |
| 3 | 98983_at | Cluster Incl U16163 Mus musculus prolyl 4-hydroxylase alpha(II)-subunit mRNA, complete cds/cds = (150,1783)/ | P | 91.1 | P | 8.6 | A | 359.6 | A |
| | | gb = U16163/gi = 836899/ug = Mm 3705/len = 2153/STRA = for | | | | | | | |
| 3 | 99044_at | Cluster Incl AI847547 UI-M-AM1-afv-f-02-0-UI.s1 Mus musculus cDNA, 3 end/clone = UI-M-AM1-afv-f-02-0-UI/ | P | 286.4 | P | 155.9 | M | 703 | A |
| | | clone_end = 3/gb = AI847547/gi = 5491453/ug = Mm 39099/len = 385/STRA = for | | | | | | | |
| 3 | 99993_at | Cluster Incl U77083 Leucine arylaminopeptidase 1, intestinal/cds = (33,2933)/gb = U77083/gi = 1674500/ug = Mm.4487/ | P | 238.7 | P | 199.3 | A | 383.8 | A |
| | | len = 3367/STRA = for | | | | | | | |
| 3 | 103959_at | Cluster Incl AI605405 vn78f02.x1 Mus musculus cDNA, 3 end/clone = IMAGE-1038075/clone_end = 3/gb = AI605405/ | P | 205.2 | P | 203 | P | 299.1 | A |
| | | gi = 4614572/ug = Mm 25582/len = 331/STRA = rev | | | | | | | |
| 3 | 93423_at | Cluster Incl AI844772 UI-M-AL1-ahq-f-11-0-UI s1 Mus musculus cDNA, 3 end/clone = UI-M-AL1-ahq-f-11-0-UI/ | A | 169.8 | P | 190.1 | A | 373.4 | P |
| | | clone_end = 3/gb = AI844772/gi = 5488678/ug = Mm.6471/len = 297/STRA = rev | | | | | | | |
| 3 | 93618_at | Cluster Incl AF026489: Beta-spectrin 3/cds = (0,47)/gb = AF026489/gi = 2511783/ug = Mm 7363/len = 696/STRA = for | P | 337.6 | P | 545.5 | P | 747.1 | P |
| 3 | 93700_at | Cluster Incl AI838022 UI-M-AP0-abi-h-01-0-UI s1 Mus musculus cDNA, 3 end/clone = UI-M-AP0-abl-h-01-0-UI/ | P | 58.3 | A | −219.9 | P | 659.7 | A |
| | | clone_end = 3/gb = AI838022/gl = 5472235/ug = Mm.8612/len = 226/STRA = for | | | | | | | |
| 3 | 97776_at | Cluster Incl X55674 M musculus mRNA for D2A dopamine receptor/cds = (129,1463)/gb = X55674/gi = 50648/ | P | 129.3 | P | 168.9 | P | 276.5 | P |
| | | ug = Mm 41970/len = 2545/STRA = rev | | | | | | | |
| 3 | 98328_at | Cluster Incl U02602: Thyroid stimulating hormone, receptor/cds = (74,2368)/gb = U02602/gi = 575923/ug = Mm 4254/ | P | 41.8 | A | 64 | P | 147.9 | P |
| | | len = 2512/STRA = for | | | | | | | |
| 3 | 93606_s_at | Cluster Incl AB021966: Mus musculus mRNA for adhesion protein RA175C, complete cds/cds = (0,920)/gb = AB021966 | P | 139.4 | M | 176.8 | M | 286.5 | P |
| | | gi = 6525254/ug = Mm 20921/len = 921/STRA = for | | | | | | | |
| 3 | 96099_at | Cluster Incl X56502 Casein kinase II, beta subunit/cds = (121,788)/gb = X56502/gi = 50420/ug = Mm 26789/len = 944/ | P | 693.2 | P | 811.2 | M | 1133.1 | P |
| | | STRA = for | | | | | | | |
| 3 | 97242_at | Cluster Incl AI849011 UI-M-AJ1-ahd-g-01-0-UI s1 Mus musculus cDNA, 3 end/clone = UI-M-AJ1-ahd-g-01-0-UI/ | A | 486.6 | P | 698.6 | A | 1018.1 | P |
| | | clone_end = 3/gb = AI849011/gi = 5492917/ug = Mm.2781/len= 467/STRA = for | | | | | | | |
| 3 | 95068_at | Cluster Incl AA763945: vv44b 12.r1 Mus musculus cDNA, 5 end/clone = IMAGE-1225247/clone_end = 5/gb = AA763945/ | A | 165.1 | P | 153.8 | A | 218.6 | P |
| | | gi = 2811467/ug = Mm.29186/len = 437/STRA = for | | | | | | | |
| 3 | 98452_at | Cluster Incl D88689: Mus musculus mRNA for fit-1, complete cds/cds = (251,4252)/gb = D88689/gi = 2809068/ug = Mm.3464/ | P | 110.9 | P | 145.4 | M | 265.9 | A |
| | | len = 6275/STRA = for | | | | | | | |
| 3 | 98482_at | Cluster Incl X78936 Parathyroid hormone receptor/cds = (56,1831)/gb = X78936/gi = 474828/ug = Mm 3542/len = 1969/ | M | 57.1 | P | 75.1 | A | 299 | P |
| | | STRA = for | | | | | | | |
| 3 | 99077_at | Cluster Incl X07750.Thyroid hormone receptor alpha/cds = (0,1232)/gb = X07750/gi = 50387/ug = Mm 4015/len = 1233/ | P | 152.4 | P | 272.4 | A | 317.4 | A |
| | | STRA = for | | | | | | | |
| 3 | 103935_at | Cluster Incl AI504474 vo44e10 Mus musculus cDNA, 3 end/clone = IMAGE-1052778/clone_end = 3/gb = AI504474/ | M | 259.7 | P | 332.7 | A | 455.5 | M |
| | | gi = 4402325/ug = Mm.25209/len = 265/STRA = rev | | | | | | | |
| 3 | 93919_at | Cluster Incl AB020741: Mus musculus NRK mRNA for NIK-related kinase, complete cds/cds = (247,4614)/gb = AB020741/ | A | 63.9 | P | 88.9 | A | 106.3 | P |
| | | gi = 6009518/ug = Mm 89850/len = 5481/STRA = for | | | | | | | |
| 3 | 95988_at | Cluster Incl C79529.C79529 Mus musculus cDNA, 3 end/clone = J0067F08/clone_end = 3/gb = C79529/gi = 2519859/ | P | 356.9 | P | 507.5 | P | 699.7 | A |
| | | ug = Mm.25039/len = 802/STRA = rev | | | | | | | |
| 3 | 98841_at | Cluster Incl M65287 Activin receptor IIA/cds = (70,1611)/gb = M65287/gi = 191663/ug = Mm 5041/len = 2563/STRA = for | A | 252.4 | P | 302.7 | A | 489.2 | P |
| 3 | 99815_at | Cluster Incl D50096 Histamine receptor H 2/cds = (0,1076)/gb = D50096/gi = 1731686/ug = Mm.57243/len = 1077/STRA = for | P | 248.3 | P | 270.5 | A | 341.9 | P |
| 3 | 101343_at | Cluster Incl AF020263: Mus musculus EWS/FLI1 activated transcript 2 (EAT-2) mRNA, complete cds/cds = (0,398)/ | P | 110.6 | A | 117.7 | A | 451.4 | P |
| | | gb = AF020263/gi = 2429353/ug = Mm.57009/len = 399/STRA = for | | | | | | | |
| 3 | 100697_at | Cluster Incl X59358 Paired box gene 3/cds = (297,1736)/gb = X59358/gi = 53591/ug = Mm 1371/len = 2347/STRA = for | P | 134.6 | P | 184.7 | A | 276.6 | P |
| 3 | AFFX- | B Subtillis Dap 5 prime | A | 90.7 | P | 101.9 | A | 119.3 | P |
| | DapX-5_at | | | | | | | | |
| 3 | 93012-at | Cluster Incl X81464: Translin/cds = (66,752)/gb = X81464/gi = 971555/ug = Mm 14644/len = 991/STRA = for | A | 97 | M | 133 | A | 254.8 | P |
| 3 | 96685_at | Cluster Incl AW121774: UI-M-BH2.3-aoe-f-12-0-UI.s1 Mus musculus cDNA, 3 end/clone = UI-M-BH2.3-aoe-f-12-0-UI/ | P | 343.3 | A | 462.1 | A | 821.4 | P |
| | | clone_end = 3/gb = AW121774/gi = 6097122/ug = Mm. 27472/len = 196/STRA = for | | | | | | | |
| 3 | 95113_at | Cluster Incl AW124257: UI-M-BH2.1-aph-g-07-0-UI.s1 Mus musculus cDNA, 3 end/clone = UI-M-BH2, 1-aph-g-07-0-UI/ | A | 44.3 | P | 14.2 | A | 143 | P |
| | | clone_end = 3/gb = AW124257/gi = 6099787/ug = Mm 29372/len = 377/STRA = for | | | | | | | |
| 3 | 101034_at | Cluster Incl U07617 Growth factor receptor bound protein 2/cds = (80,733)/gb = U07617/gi = 464004/ug = Mm 6900/ | P | 265 | P | 342.6 | M | 456.4 | A |
| | | len = 2362/STRA = for | | | | | | | |
| 3 | 99511_at | Cluster Incl X53532 Protein kinase C, beta/cds = (48,2069)/gb = X53532/gi = 53692/ug = Mm 4182/len = 2974/STRA = for | P | 37.5 | P | 81.7 | A | 260.4 | A |
| 3 | 102958_at | Cluster Incl U82441 Early B-cell factor 2/cds = (23,1750)/gb = U82441/gi = 3757777/ug = Mm 179/len = 1844/STRA = for | P | 290.3 | P | 403 | A | 541.4 | P |
| 3 | 103240_f_at | Cluster Incl AF017258 Mus musculus ribonuclease 3 precursor (mR-3) gene, complete cds/cds = (0,470)/gb = AF017258/ | A | 179.6 | P | 171.8 | A | 372.3 | A |
| | | gi = 2407237/ug = Mm 20370/len = 471/STRA = for | | | | | | | |
| 3 | 103498_at | Cluster Incl L38613 Mus musculus glucagon receptor mRNA, complete cds/cds = (94,1551)/gb = L38613/gi = 603463/ | P | 383.3 | P | 455.7 | P | 579.3 | M |
| | | ug = Mm 22329/len = 1944/STRA = for | | | | | | | |
| 3 | 103700_r_at | Cluster Incl AI646838 mt86b06.y1 Mus musculus cDNA, 5 end/clone = IMAGE-636755/clone_end = 5/gb = AI646638/ | P | 278.6 | M | 370.6 | M | 488.4 | M |
| | | gi = 4725113/ug = Mm 24029/len = 294/STRA = rev | | | | | | | |
| 3 | 104421_at | Cluster Incl U87147 Mus musculus flavin-containing monooxygenase 3 mRNA, complete cds/cds = (37,1641)/gb = U87147/ | A | 106.4 | A | 153.4 | P | 252.7 | P |
| | | gi = 1841861/ug = Mm 2900/len = 2020/STRA = for | | | | | | | |
| 3 | 92977_s_at | Cluster Incl X75330 H6 homeo box 3/cds = (307,1683)/gb = X75330/gi = 406586/ug = Mm 5016/len = 1972/STRA = for | M | 79.6 | A | 169.6 | P | 216.7 | P |
| 3 | 93710_at | Cluster Incl M37759: Mouse serine 1 ultra high sulfur protein gene, complete cds/cds = (0,692)/gb = M37759/gi = 200961/ | P | 495.3 | A | 516.2 | P | 1045.6 | A |
| | | ug = Mm 86481/len = 693/STRA = for | | | | | | | |
| 3 | 96533_at | Cluster Incl AI508931 vc48e10 y1 Mus musculus cDNA, 5 end/clone = IMAGE-777834/clone_end = 5/gb = AI508931/ | P | 107.8 | P | 119.3 | A | 163.2 | A |
| | | gi = 4407836/ug = Mm 27464/len = 318/STRA = for | | | | | | | |
| 3 | 96583_at | Cluster Incl AI414051 ma05b11.x1 Mus musculus cDNA, 3 end/clone = IMAGE-303645/clone_end = 3/gb = AI414051/ | P | 120.8 | P | 101.5 | A | 181.7 | A |
| | | gi = 4257555/ug = Mm 29393/len = 473/STRA = rev | | | | | | | |
| 3 | 101333_at | Cluster Incl U73819 Mus musculus polypeptide GalNAc transferase-T4 (ppGaNTase-T4) mRNA, complete cds/ | P | 23.1 | A | 4.9 | A | 39 | P |
| | | cds = (0,1736)/gb = U73819/gi = 2121219/ug = Mm 56991/len = 1737/STRA = for | | | | | | | |
| 3 | 96968_at | Cluster Incl AB024521 Mus musculus mRNA for limitin, complete cds/cds = (160,708)/gb = AB024521/gi = 5821277/ | A | 337.1 | M | 535.3 | P | 607 | P |
| | | ug = Mm 88804/len = 997/STRA = for | | | | | | | |
| 4 | AFFX- | E Coli BioC 3 prime | P | 111.3 | P | 1500.2 | P | 1133.6 | P |
| | BioC-3_at | | | | | | | | |
| 4 | 92547_at | Cluster Incl AB011081 Mus musculus mRNA for huntingtin interacting protein-2, complete cds/cds = (161,763)/ | P | 634.1 | P | 905.1 | P | 430.1 | P |
| | | gb = AB011081/gi = 2897817/ug = Mm.10082/len = 955/STRA = for | | | | | | | |
| 4 | 92622_at | Cluster Incl AI851064 UI-M-BH0-ajv-h-03-0-UI a1 Mus musculus cDNA, 3 end/clone = UI-M-BH0-ajv-h-03-0-UI/ | P | 466.7 | P | 689.1 | P | 500 | P |
| | | clone_end = 3/gb = AI851064/gi = 5494970/ug = Mm 11112/len = 316/STRA = for | | | | | | | |
| 4 | 92782_at | Cluster Incl U39074 Thymopoietin/cds = (237,1595)/gb = U39074/gi = 1335840/ug = Mm.124/len = 2170/STRA = for | P | 106.1 | P | 292.4 | P | 240.8 | P |
| 4 | 92808_f_at | Cluster Incl X17069 Mouse mRNA for protein with homology to transition protein 2 (TP2)/cds = (123,1358)/gb = X17069/ | P | 888.5 | P | 1375.9 | P | 707.5 | P |
| | | gi = 54875/ug = Mm 12758/len = 2026/STRA = for | | | | | | | |
| 4 | 92850_at | Cluster Incl AI836446 UI-M-AI0-aak-d-12-0-UI s1 Mus musculus cDNA, 3 end/clone = UI-M-AI0-aak-d-12-0-UI/ | P | 953.6 | P | 1189.9 | P | 955.7 | P |
| | | clone_end = 3/gb = AI836446/gi = 5470659/ug = Mm 13705/len = 451/STRA = for | | | | | | | |
| 4 | 93014_at | Cluster Incl Y17223 Mus musculus mRNA for F1F0-ATP synthase, g subunit/cds = (11,322)/gb = Y17223/gi = 3123645/ | P | 3459.2 | P | 4518.1 | P | 3674.7 | P |
| | | ug = Mm 14663/len = 420/STRA = for | | | | | | | |
| 4 | 93028_at | Cluster Incl X58196 H19 fetal liver mRNA/cds = UNKNOWN/gb = X58196/gi = 51131/ug = Mm 14802/len = 1899/STRA = for | P | 2457.5 | P | 4934.4 | P | 2881.4 | P |
| 4 | 93064_at | Cluster Incl AF035207 Mus musculus Nip21 mRNA, complete cds/cds = (133,1113)/gb = AF035207/gi = 2911347/ | P | 244.6 | P | 323 | P | 193.7 | P |
| | | ug = Mm.1561/len = 2213/STRA = for | | | | | | | |
| 4 | 93293_at | Cluster Incl M27844 Calmodulin/cds = (0,449)/gb = M27844/gi = 192364/ug = Mm 18041/len = 450/STRA = for | P | 3783.2 | P | 5600.9 | P | 4864.7 | P |
| 4 | 93306_at | Cluster Incl U51196: Mus musculus APC-binding protein EB1 homolog mRNA, complete cds/cds = (66,872)/gb = U51196/ | P | 1127.1 | P | 1427.1 | P | 1175.2 | P |
| | | gi = 1256433/ug = Mm.18418/len = 1203/STRA = for | | | | | | | |
| 4 | 93354_at | Cluster Incl Z22661 Apolipoprotein Cl/cds = (49,315)/gb = Z22661/gi = 410495/ug = Mm 189/len = 408/STRA = for | P | 975.8 | P | 1605 | P | 977.6 | P |
| 4 | 93365_s_at | Cluster Incl AI838149 UI-M-AL.0-abw-e-03-0-UI s1 Mus musculus cDNA, 3 end/clone = UI-M-AL0-abw-e-03-0-UI/ | P | 493.5 | P | 735.9 | P | 470.8 | P |
| | | clone_end = 3/gb = AI838149/gi = 5472362/ug = Mm 18972/len = 402/STRA = for | | | | | | | |
| 4 | 93366_r_at | Cluster Incl AI838149 UI-M-AL.0-abw-e-03-0-UI s1 Mus musculus cDNA, 3 end/clone = UI-M-AL0-abw-e-03-0-UI/ | P | 630.4 | P | 847 | P | 702.9 | P |
| | | clone_end = 3/gb = AI838149/gi = 5472362/ug = Mm 18972/len = 402/STRA = for | | | | | | | |
| 4 | 93579_at | Cluster Incl AW121613 UI-M-BH2 2-aoq-d-12-0-UI s1 Mus musculus cDNA, 3 end/clone = UI-M-BH2 2-aoq-d-12-0-UI/ | P | 625.4 | P | 834.5 | P | 556.1 | P |
| | | clone_end = 3/gb = AW121613/gi = 6096946/ug = Mm 2053/len = 135/STRA = for | | | | | | | |
| 4 | 93800_f_at | Cluster Incl AB013607 Mus musculus mRNA for c29, complete cds/cds = (21,1367)/gb = AB013607/gi = 3868801/ | P | 2628.1 | P | 5173.6 | P | 3767.9 | P |
| | | ug = Mm 21408/len = 1539/STRA = for | | | | | | | |
| 4 | 93810_at | Cluster Incl X68378 Cathepsin D/cds = (0,1232)/gb = X68378/gi = 50302/ug = Mm 2147/len = 1233/STRA = for | P | 1805 | P | 2860.5 | P | 1641.8 | P |
| 4 | 93822_at | Cluster Incl X73331 M musculus mRNA for ribosomal protein L37a/cds = (18,296)/gb = X73331/gi = 312413/ug = Mm 21529/ | P | 952 | P | 1621.2 | P | 1490.9 | P |
| | | len = 349/STRA = for | | | | | | | |
| 4 | 93844_at | Cluster Incl AW061302 UI-M-BH1-anw-c-09-0-UI s1 Mus musculus cDNA, 3 end/clone = UI-M-BH1-anw-c-09-0-UI/ | P | 3110.8 | P | 4615.5 | P | 3127.5 | P |
| | | clone_end = 3/gb = AW061302/gi = 6009053/ug = Mm 21625/len = 321/STRA = for | | | | | | | |
| 4 | 93980_at | Cluster Incl AW124520 UI-M-BH2, 1-apo-f-11-0-UI.s1 Mus musculus cDNA, 3 end/clone = UI-M-BH2 1-apo-f-11-0-UI/ | P | 152.4 | P | 188.3 | P | 126.3 | P |
| | | clone_end = 3/gb = AW124520/gi = 6100050/ug = Mm 21693/len = 511/STRA = for | | | | | | | |
| 4 | 93987_f_at | Cluster Incl AW121568: UI-M-BH2 2-aop-g-06-0-UI s1 Mus musculus cDNA, 3 end/clone = UI-M-BH2 2-aop-g-06-0-UI/ | P | 4048.3 | P | 5814.1 | P | 4830 | P |
| | | clone_end = 3/gb = AW121568/gi = 6096901/ug = Mm 21724/len = 329/STRA = for | | | | | | | |
| 4 | 94034_at | Cluster Incl AI839882: UI-M-AH0-acw-b-11-0-UI s1 Mus musculus cDNA, 3 end/clone = UI-M-AH0-acw-b-11-0-UI/ | P | 393.3 | P | 538.3 | P | 358.9 | P |
| | | clone_end = 3/gb = AI839882/gi = 5474095/ug = Mm 21911/len = 353/STRA = for | | | | | | | |
| 4 | 94042_f_at | Cluster Incl AI843937: UI-M-AP1-agh-b-10-0-UI a1 Mus musculus cDNA, 3 end/clone = UI-M-AP1-agh-b-10-0-UI/ | P | 3841.9 | P | 4966.2 | P | 4271.8 | P |
| | | clone_end = 3/gb = AI843937/gi = 5478150/ug = Mm 21955/len = 356/STRA = for | | | | | | | |
| 4 | 94056_at | Cluster Incl M21285 Stearoyl-coenzyme A desaturase 1/cds = (0,1067)/gb = M21285/gi = 200949/ug = Mm 22026/len = 1068/ | P | 2612.1 | P | 5305.7 | P | 1910.6 | P |
| | | STRA = for | | | | | | | |
| 4 | 94057_g_at | Cluster Incl M21285: Stearoyl-coenzyme A desaturase 1/cds = (0,1067)/gb = M21285/gi = 200949/ug = Mm.22026/len = 1068/ | P | 1517.6 | P | 5541 | P | 922.2 | P |
| | | STRA = for | | | | | | | |
| 4 | 94218_at | Cluster Incl M20130: Mouse t complex polypeptide 1 (Tcp-1-a) mRNA, 3 end/cds = (0,714)/gb = M20130/gi = 201722/ | P | 316.8 | P | 481 | P | 371.8 | P |
| | | ug = Mm 2223/len = 863/STRA = for | | | | | | | |
| 4 | 94276_at | Cluster Incl AF064635: Mus musculus putative ateroid dehydrogenase (KIK-I) mRNA, complete cds/cds = (19,957)/ | P | 575 | P | 1311.7 | 876.9 | P | 761.5 |
| | | gb = AF064635/gi = 3142701/ug = Mm.22505/len = 978/STRA = for | | | | | | | |
| 4 | 94312_at | Cluster Incl AI180784: ub76c07.r1 Mus musculus cDNA, 5 and/clone = IMAGE-1383660/clone_end = 5/gb = AI180784/ | P | 473.3 | P | 796.6 | P | 541.5 | P |
| | | gi = 3731422/ug = Mm 22635/len = 360/STRA = rev | | | | | | | |
| 4 | 94819_f_at | Cluster Incl AF005886: Mus musculus cyclin I mRNA, complete cds/cds = (286,1419)/gb = AF005888/gi = 4101686/ | P | 1372.4 | | 2127.6 | P | 1581.4 | P |
| | | ug = Mm 22711/len = 1548/STRA = for | | | | | | | |
| 4 | 95437_at | Cluster Incl AI836139 UI-M-AQ0-aai-b-06-0-UI.a2 Mus musculus cDNA, 3 end/clone = UI-M-AQ0-aai-b-06-0-UI/ | P | 513.5 | P | 585.4 | P | 466.6 | P |
| | | clone_end = 3/gb = AI836139/gi = 5470352/ug = Mm 24538/len = 395/STRA = for | | | | | | | |
| 4 | 95442_at | Cluster Incl AI835706: UI-M-AIO-aan-b-08-0-UI s1 Mus musculus cDNA, 3 end/clone = UI-M-AI0-aan-b-08-0-UI/ | P | 407.4 | P | 602.5 | P | 435.6 | P |
| | | clone_end = 3/gb = AI835706/gi = 5469919/ug = Mm 24571/len = 329/STRA = for | | | | | | | |
| 4 | 95457_at | Cluster Incl AI854214 UI-M-BH0-ajz-e-09-0-UI s1 Mus musculus cDNA, 3 end/clone = UI-M-BH0-ajz-e-09-0-UI/ | P | 794.7 | P | 900.6 | P | 662.9 | P |
| | | clone_end = 3/gb = AI854214/gi = 5498120/ug = Mm 24703/len = 647/STRA = for | | | | | | | |
| 4 | 95470_at | Cluster Incl AI846025: UI-M-AP1-agi-g-04-0-UI s1 Mus musculus cDNA, 3 end/clone = UI-M-AP1-agi-g-04-0-UI/ | P | 450.7 | P | 506.2 | P | 417.1 | P |
| | | clone_end = 3/gb = AI846025/gi = 5489931/ug = Mm 24789/len = 451/STRA = for | | | | | | | |
| 4 | 96027_at | Cluster Incl AW120546: UI-M-BH1-ana-e-02-0-UI.s2 Mus musculus cDNA, 3 end/clone = UI-M-BH1-ana-e-02-0-UI/ | P | 240 | P | 331.4 | P | 192.6 | P |
| | | clone_end = 3/gb = AW120546/gi = 6095879/ug = Mm 25732/len = 473/STRA = for | | | | | | | |
| 4 | 96050_at | Cluster Incl AJ011740 Mus musculus mRNA for integrase interacter 1a protein (INI1A)/cds = (193,1350)/gb = AJ011740/ | P | 526.7 | P | 736.1 | P | 589.3 | P |
| | | gi = 4138222/ug = Mm 26116/len = 1622/STRA = for | | | | | | | |
| 4 | 96068_at | Cluster Incl AI848821: UI-M-AJ1-ahb-a-11-0-UI s1 Mus musculus cDNA, 3 end/clone = UI-M-AJ1-ahb-a-11-0-UI/ | P | 315.4 | P | 403.4 | P | 317.8 | P |
| | | clone_end = 3/gb = AI848821/gi = 5492727/ug = Mm 26418/len = 401/STRA = for | | | | | | | |
| 4 | 96653_at | Cluster Incl AI851762: UI-M-BH0-aim-d-03-0-UI.s1 Mus musculus cDNA, 3 end/clone = UI-M-BH0-aim-d-03-0-UI/ | P | 2086.9 | P | 4014.7 | P | 1766.4 | P |
| | | clone_end = 3/gb = AI851762/gi = 5495668/ug = Mm 27336/len = 419/STRA = for | | | | | | | |
| 4 | 96674_at | Cluster Incl AW123553: UI-M-BH2 1-aqb-f-12-0-UI s1 Mus musculus cDNA, 3 end/clone = UI-M-BH2 1-aqb-f-12-0-UI/ | P | 1089 | P | 1287.6 | P | 900.9 | P |
| | | clone_end = 3/gb = AW123553/gi = 6099083/ug = Mm 27404/len = 400/STRA = for | | | | | | | |
| 4 | 96677_at | Cluster Incl AI849566: UI-M-AH1-agr-c-04-0-UI s1 Mus musculus cDNA, 3 end/clone = UI-M-AH1-agr-c-04-0-UI/ | P | 748.4 | P | 951 | P | 843.8 | P |
| | | clone_end = 3/gb = AI849566/gi = 5493472/ug = Mm 27424/len = 351/STRA = for | | | | | | | |
| 4 | 96684_at | Cluster Incl AI846123: UI-M-AN1-afj-a-11-0-UI s2 Mus musculus cDNA, 3 end/clone = UI-M-AN1-afj-a-11-0-UI/ | P | 304.7 | P | 645.6 | P | 253.6 | P |
| | | clone_end = 3/gb = AI846123/gi = 5490029/ug = Mm 27453/len = 341/STRA = for | | | | | | | |
| 4 | 97206_at | Cluster Incl AW230369: uo62g07 y1 Mus musculus cDNA, 5 end/clone = IMAGE-2647164/clone_end = 5/gb = AW230369/ | P | 464.5 | P | 759.7 | P | 532.7 | P |
| | | gi = 6559685/ug = Mm 27700/len = 692/STRA = for | | | | | | | |
| 4 | 98894_at | Cluster Incl AA867655 vx12e03 r1 Mus musculus cDNA, 5 end/clone = IMAGE-1264252/clone_end = 5/gb = AA867655/ | P | 729 | P | 922.8 | P | 634.9 | P |
| | | gi = 2963100/ug = Mm 28550/len = 428/STRA = for | | | | | | | |
| 4 | 98901_at | Cluster Incl AW047870: UI-M-BH1-afs-f-10-0-UI s1 Mus musculus cDNA, 3 end/clone = UI-M-BH1-afs-f-10-0-UI/ | P | 510.8 | P | 708.7 | P | 564.3 | P |
| | | clone_end = 3/gb = AW047870/gi = 5908399/ug = Mm 28578/len = 353/STRA = for | | | | | | | |
| 4 | 94456_at | Cluster Incl AI841497: UI-M-AH0-acy-d-12-0-UI s1 Mus musculus cDNA, 3 end/clone = UI-M-AH0-acy-d-12-0-UI/ | P | 1442.2 | P | 2134.9 | P | 1449.6 | P |
| | | clone_end = 3/gb = AI841497/gi = 5475710/ug = Mm 28805/len = 411/STRA = for | | | | | | | |
| 4 | 94466_f_at | Cluster Incl U19891 CCAAT/enhancer binding protein alpha (C/EBP), related sequence 1/cds = (11,3004)/gb = U19891/ | P | 423.5 | P | 537.1 | P | 452.9 | P |
| | | gi = 790574/ug = Mm 28845/len = 3302/STRA = for | | | | | | | |
| 4 | 94528_at | Cluster Incl AI846206 UI-M-AM1-agb-e-11-0-UI s1 Mus musculus cDNA, 3 end/clone = UI-M-AM1-agb-e-11-0-UI/ | P | 249.9 | P | 388.9 | P | 287.2 | P |
| | | clone_end = 3/gb = AI846206/gi = 5490112/ug = Mm 29037/len = 141/STRA = for | | | | | | | |
| 4 | 94548_at | Cluster Incl AI790537 ul02d10 x1 Mus musculus cDNA, 3 end/clone = IMAGE-2064883/clone_end = 3/gb = AI790537/ | P | 701.6 | P | 1212.8 | P | 620.1 | P |
| | | gi = 5338253/ug = Mm.29078/len = 440/STRA = for | | | | | | | |
| 4 | 95058_f_at | Cluster Incl AW121984 UI-M-BH2 3-aoj-b-10-0-UI s1 Mus musculus cDNA, 3 end/clone = UI-M-BH2 3-aoj-b-10-0-UI/ | P | 1711.7 | P | 2395.4 | P | 1725.7 | P |
| | | clone_end = 3/gb = AW121984/gi = 6097345/ug = Mm.29153/len = 390/STRA = for | | | | | | | |
| 4 | 95118_r_at | Cluster Incl AI131895 ue97e08 x1 Mus musculus cDNA, 3 end/clone = IMAGE-1499078/clone_end = 3/gb = AI131895/ | P | 699.4 | P | 958.3 | P | 649.8 | P |
| | | gi = 3601911/ug = Mm.29381/len = 397/STRA = for | | | | | | | |
| 4 | 95686_at | Cluster Incl AW047017 UI-M-BH1-alp-h-05-0-UI s1 Mus musculus cDNA, 3 end/clone = UI-M-BH1-alp-h-05-0-UI/ | P | 213.6 | P | 345.1 | P | 213 | P |
| | | clone_end = 3/gb = AW047017/gi = 5907546/ug = Mm.29641/len = 353/STRA = for | | | | | | | |
| 4 | 95702_at | Cluster Incl AI461803 uc43c04.x1 Mus musculus cDNA, 3 end/clone = IMAGE-1400742/clone_end = 3/gb = AI461803/ | P | 846.3 | P | 1312.1 | P | 1030.9 | P |
| | | gi = 4315833/ug = Mm.29697/len = 481/STRA = for | | | | | | | |
| 4 | 95744_at | Cluster Incl U13837: Mus musculus vacuolar adenosine triphosphatase subunit A gene, complete cds/cds = (0,1853)/ | P | 516.1 | P | 1085.3 | P | 540.1 | P |
| | | gb = U13837/gi = 1184658/ug = Mm.29771/len = 1854/STRA = for | | | | | | | |
| 4 | 95745_g_at | Cluster Incl U13837 Mus musculus vacuolar adenosine triphosphatase subunit A gene, complete cds/cds = (0,1853)/ | P | 578.7 | P | 807.9 | P | 679 | P |
| | | gb = U13837/gi = 1184658/ug = Mm.29771/len = 1854/STRA = for | | | | | | | |
| 4 | 95749_at | Cluster Incl AW122364 UI-M-BH2.2-aow-a-11-0-UI.s1 Mus musculus CDNA, 3 end/clone = UI-M-BH2.2-aow-a-11-0-UI/ | P | 285.4 | P | 594.7 | P | 203.1 | P |
| | | clone_end = 3/gb = AW122364/gi = 6097859/ug = Mm 29778/len = 488/STRA = for | | | | | | | |
| 4 | 95758_at | Cluster Incl M26270 Stearoyl-coenzyme A desaturase 2/cds = (300,1376)/gb = M26270/gi = 200933/ug = Mm.298/len = 5032/ | P | 1889 | P | 3347 | P | 2485.8 | P |
| | | STRA = for | | | | | | | |
| 4 | 96270_at | Cluster Incl AI847092: UI-M-AP1-agn-e-07-0-U1 s1 Mus musculus CDNA, 3 end/clone = UI-M-AP1-agn-e-07-0-UI/ | P | 1491.4 | P | 1971.6 | P | 1256 | P |
| | | clone_end = 3/gb = AI847092/gi = 5490998/ug = Mm 29852/len = 261/STRA = for | | | | | | | |
| 4 | 96337_at | Cluster Incl AF033350: Mus musculus CDCREL-1 homolog (Cdcrel-1) MRNA, partial cds/cds = (0,1044)/gb = AF033350/ | P | 344.1 | P | 544.9 | P | 427.2 | P |
| | | gi = 3986408/ug = Mm 29976/len = 1973/STRA = for | | | | | | | |
| 4 | 96343_at | Cluster Incl AI836968 UI-M-AJ0-aaz-c-09-0-UI s1 Mus musculus CDNA, 3 end/clone = UI-M-AJ0-aaz-c-09-0-UI/ | P | 1494.6 | P | 2617 | P | 1600.2 | P |
| | | clone_end = 3/gb = AI836968/gi = 5471181/ug = Mm.29991/len = 445/STRA = for | | | | | | | |
| 4 | 96849_at | Cluster Incl AA655369: vs24h12 r1 Mus musculus CDNA, 5 end/clone = IMAGE-1139207/clone_end = 5/gb = AA655369/ | P | 318.6 | P | 398.6 | P | 270.7 | P |
| | | gi = 2591523/ug = Mm 30034/len = 545/STRA = for | | | | | | | |
| 4 | 96943_at | Cluster Incl AW125234 UI-M-BH2 1-apa-e-0300-UI s1 Mus musculus cDNA, 3 end/clone = UI-M-BH2 1-apa-e-03-0-UI/ | P | 712.6 | P | 1127.5 | P | 679.4 | P |
| | | clone_end = 3/gb = AW125234/gi = 6100764/ug = Mm 30195/len = 310/STRA = for | | | | | | | |
| 4 | 97456_at | Cluster Incl AI838021: UI-M-AP0-abl-g-11-0-UI.s1 Mus musculus cDNA, 3 end/clone = UI-M-AP0-abl-g-11-0-UI/ | P | 517.4 | P | 861.8 | P | 507.6 | P |
| | | clone_end = 3/gb = AI838021/gi = 5472234/ug = Mm.30263/len = 392/STRA = for | | | | | | | |
| 4 | 97459_at | Cluster Incl AI851441: UI-M-BH0-akj-e-05-0-UI.s1 Mus musculus cDNA, 3 end/clone = UI-M-BH0-akj-e-05-0-UI/ | P | 1603.7 | P | 1740.7 | P | 1607.5 | P |
| | | clone_end = 3/gb = AI851441/gi = 5495347/ug = Mm.30270/len = 338/STRA = for | | | | | | | |
| 4 | 97497_at | Cluster Incl Z11886 Notch gene homolog 1, (Drosphila)/cds = (78,7673)/gb = Z11886/gi = 288502/ug = Mm.31255/len = 8064/ | P | 743.5 | P | 1242.2 | P | 780.8 | P |
| | | STRA = for | | | | | | | |
| 4 | 97504_at | Cluster Incl M83749 Cyclin D2/cds = (270,1139)/gb = M83749/gi = 192938/ug = Mm 3141/len = 1255/STRA = for | P | 811.6 | P | 2061 | P | 1332.3 | P |
| 4 | 97512_at | Cluster Incl AW226650 um58g10.y1 Mus musculus cDNA, 5 end/clone = IMAGE-2270274/clone_end = 5/gb = AW226650/ | P | 1934 | P | 2708.5 | P | 2220.6 | P |
| | | gi = 6555946/ug = Mm 318/len = 390/STRA = for | | | | | | | |
| 4 | 98053_at | Cluster Incl AF058797 Mus musculus 14-3-3 protein beta mRNA, complete cds/cds = (171,911)/gb = AF058797/gi = 3065924/ | P | 1379.7 | P | 2201.8 | P | 1293.1 | P |
| | | ug = Mm 34319/len = 1022/STRA = for | | | | | | | |
| 4 | 98056_at | Cluster Incl AI846214 UI-M-AM1-agb-f-10-0-UI s1 Mus musculus cDNA, 3 end/clone = UI-M-AM1-agb-f-10-0-UI/ | P | 960.8 | P | 1543.3 | P | 1293.5 | P |
| | | clone _end = 3/gb = AI846214/gi = 5490120/ug = Mm 34346/len = 343/STRA = for | | | | | | | |
| 4 | 98077_at | Cluster Incl AA796831: vp15g02 r1 Mus musculus cDNA, 5 end/clone = IMAGE-1068722/clone_end = 5/gb = AA796831/ | P | 144.3 | P | 296.6 | P | 191.3 | P |
| | | gi = 2859786/ug = Mm 34549/len = 424/STRA = for | | | | | | | |
| 4 | 98111_at | Cluster Incl L40406 Heat shock protein, 105 kDa/cds = (139,2718)/gb = L40406/gi = 840651/ug = Mm 34828/len = 3387/ | P | 456.2 | P | 626.3 | P | 274.8 | P |
| | | STRA = for | | | | | | | |
| 4 | 98112_r_at | Cluster Incl AI839225 UI-M-AK0-adh-d-01-0-UI s1 Mus musculus cDNA, 3 end/clone = UI-M-AK0-adh-d-01-0-UI/ | P | 302.1 | P | 503.7 | P | 242.8 | P |
| | | clone_end = 3/gb = AI839225/gi = 5473438/ug = Mm 34832/len = 429/STRA = for | | | | | | | |
| 4 | 98113_at | Cluster Incl U60824: Mus musculus proteasome beta-subunit C5 (Pamb1) mRNA, allele B, complete cds/cds = (29,751)/ | P | 1890.2 | P | 2788.1 | P | 2426.8 | P |
| | | gb = U60824/gi = 1698583/ug = Mm 34837/len = 812/STRA = for | | | | | | | |
| 4 | 98122_at | Cluster Incl AF074600 Mus musculus LIM domain transcription factor LMO4 mRNA, complete cds/cds = (0,497)/ | P | 1636 | P | 2403 | P | 1535.3 | P |
| | | gb = AF074600/gi = 3702853/ug = Mm.35004/len = 498/STRA = for | | | | | | | |
| 4 | 98532_at | Cluster Incl AF011644 Mus musculus oral tumuor suppressor homolog (Doc-1) mRNA, partial cds/cds = (0,330)/ | P | 1355.9 | P | 1635.7 | P | 1359.5 | P |
| | | gb = AF011644/gi = 2305225/ug = Mm 35847/len = 981/STRA = for | | | | | | | |
| 4 | 98555_at | Cluster Incl AB008516: Tetratricopeptide repeat domain/cds = (11,5950)/gb = AB008516/gi = 3308963/ug = Mm 3679/ | P | 447.1 | P | 515.7 | P | 482.1 | P |
| | | len = 7172/STRA = for | | | | | | | |
| 4 | 98602_at | Cluster Incl U08110 RAN GTPase activating protein 1/cds = (274,2043)/gb = U08110/gi = 472851/ug = Mm 3833/len = 2981/ | P | 477.7 | P | 726.2 | P | 369.1 | P |
| | | STRA = for | | | | | | | |
| 4 | 99098_at | Cluster Incl AW045533 UI-M-BH1-aks-c-08-0-UI s1 Mus musculus cDNA, 3 end/clone = UI-M-BH1-aks-c-08-0-UI/ | P | 710.5 | P | 1310.7 | P | 775.5 | P |
| | | clone_end = 3/gb = AW045533/gi = 5906062/ug = Mm 39472/len = 426/STRA = for | | | | | | | |
| 4 | 99128_at | Cluster Incl AI849767 UI-M-AL1-ahn-f-09-0-UI s1 Mus musculus cDNA, 3 end/clone = UI-M-AL1-ahn-f-09-0-UI/ | P | 2079.6 | P | 3330.4 | P | 2720.8 | P |
| | | clone_end = 3/gb = AI849767/gi = 5493673/ug = Mm 41/len = 362/STRA = for | | | | | | | |
| 4 | 99147_at | Cluster Incl AI843686 UI-M-AP1-agh-05-0-UI s1 Mus musculus cDNA, 3 end/clone = UI-M-AP1-agh-g-05-0-UI/ | P | 1152.7 | P | 1534.8 | P | 697.1 | P |
| | | clone_end = 3/gb = AI843986/gi = 5478199/ug = Mm 41486/len = 246/STRA = for | | | | | | | |
| 4 | 99569_at | Cluster Incl AF021836 Mus muscules hair keratin basic 5 (Hb5) mRNA, complete cds/cds = (61,1584)/gb = AF021836/ | P | 947.9 | P | 2623.3 | P | 1389.6 | P |
| | | gi = 4103155/ug = Mm 42251/len = 2344/STRA = for | | | | | | | |
| 4 | 99583_at | Cluster Incl X53451 Glutathione S-transferase, pi 2/cds = (8,640)/gb = X53451/gi = 51124/ug = Mm 426/len = 703/STRA = for | P | 1269.7 | P | 1881.1 | P | 1276.8 | P |
| 4 | 99607_at | Cluster Incl Z47088: Transcription elongation factor B (SIII), polypeptide 1 (15 KDa),-like/cds = UNKNOWN/gb = Z47088/ | P | 1176.3 | P | 1546 | P | 1051.1 | P |
| | | gi = 861057/ug = Mm.42944/len = 1219/STRA = for | | | | | | | |
| 4 | 99631_f_at | Cluster Incl U08440 Mus musculus Balb/c cytochrome c oxidase subunit VIaL mRNA, complete cds/cds = (72,332)/ | P | 3476.3 | P | 4478.5 | P | 3301 | P |
| | | gb = U08440/gi = 475706/ug = Mm.43415/len = 521/STRA = for | | | | | | | |
| 4 | 99672_at | Cluster Incl Z49916: M.muscules mRNA for CLCN4/cds = (377,2620)/gb = Z49916/gi = 929679/ug = Mm 4408/len = 2739/ | P | 227.7 | P | 323.9 | P | 218.7 | P |
| | | STRA = for | | | | | | | |
| 4 | 99674_at | Cluster Incl AW060297 UI-M-BH1-amv-c-04-0-UI s1 Mus musculus cDNA, 3 end/clone = UI-M-BH1-amv-c-04-0-UI/ | P | 505.1 | P | 746.3 | P | 501.8 | P |
| | | clone_end = 3/gb = AW060297/gi = 6008013/ug = Mm 44174/len = 415/STRA = for | | | | | | | |
| 4 | 100066_at | Cluster Incl U20892 Phoshonbosylglycinamide formyltransferase/cds = (115,3147)/gb = U20892/gi = 2182233/ug = Mm 4505/ | P | 454.2 | P | 640.4 | P | 546.3 | P |
| | | len = 3296/STRA = for | | | | | | | |
| 4 | 100091_at | Cluster Incl D87990 House mouse, Musculus domesticus mRNA for UDP-galactose transporter related isozyme 1, | P | 1024.2 | P | 1403.9 | P | 1167.4 | P |
| | | complete cds/cds = (88,1056)/gb = D87990)/gi = 1694638/ug = Mm.4593/len = 1169/STRA = for | | | | | | | |
| 4 | 100550_f_at | Cluster Incl AW060422: UI-M-BH1-anj-c-07-0-UI s1 Mus musculus cDNA, 3 end/clone = UI-M-BH1-anj-c-07-0-UI/ | P | 3646.9 | P | 4542.5 | P | 3560.7 | P |
| | | clone_end = 3/gb = AW060422/gi = 6008173/ug = Mm 548/len = 373/STRA = for | | | | | | | |
| 4 | 100567_at | Cluster Incl M20497 Mouse adipose fatty acid binding protein (422) gene, 5 end/cds = (65,97)/gb = M20497/gi = 191743/ | P | 1676.5 | P | 2449.1 | P | 1565.1 | P |
| | | ug = Mm 582/len = 98/STRA = for | | | | | | | |
| 4 | 101044_at | Cluster Incl X13752 Delta-aminolevulinate dehydratase/cds = (6,998)/gb = X13752/gi = 52951/ug = Mm 6988/len = 1080/ | P | 537.6 | P | 1390.6 | P | 524.2 | P |
| | | STRA = for | | | | | | | |
| 4 | 101051_at | Cluster Incl AF004941 Mus musculus S100A3 calcuim binding protein mRNA, complete cds/cds = (59,364)/gb = AF004941/ | P | 1637.9 | P | 4408.7 | P | 2281.7 | P |
| | | gi = 3916049/ug = Mm 703/len = 712/STRA = for | | | | | | | |
| 4 | 101052_g_at | Cluster Incl AF004941 Mus musculus S100A3 calcuim binding protein mRNA, complete ccds/cds = (59,364)/gb = AF004941/ | P | 778.6 | P | 3017.3 | P | 1041.7 | P |
| | | gi = 3916049/ug = Mm 703/len = 712/STRA = for | | | | | | | |
| 4 | 101530_at | Cluster Incl U97079 Mus musculus US-116 KD mRNA, complete cds/cds = (27,2942)/gb = U97079/gi = 2276461/ug = Mm 873/ | P | 446.1 | P | 669.4 | P | 401.1 | P |
| | | len = 3271/STRA = for | | | | | | | |
| 4 | 101561_at | Cluster Incl K02236 Metallothionein 2/cds = (0,185)/gb = K02236/gi = 199131/ug = Mm.89170/len = 186/STRA = for | P | 3228 | P | 5744.4 | P | 2978.1 | P |
| 4 | 101569_at | Cluster Incl AB033744 Mus musculus mRNA for type II cytokeratin, complete cds/cds = (51,1625)/gb = AB0033744/ | P | 2575.8 | P | 5802.1 | P | 3358.8 | P |
| | | gi = 6092074/ug = Mm 89769/len = 2193/STRA = for | | | | | | | |
| 4 | 101581_at | Cluster Incl U82122.Ubiqutin conjugating enzyme E3A/cds = (0,2549)/gb = U82122/gi = 1843534/ug = Mm 9002/len = 2550/ | P | 281.4 | P | 432.8 | P | 309 | P |
| | | STRA = for | | | | | | | |
| 4 | 101984_at | Cluster Incl AF004591 Mus musculus copper transport protein Atox1 (ATOX1) mRNA, complete cds/cds = (38,244)/ | P | 2524.6 | P | 3432.8 | P | 2719.1 | P |
| | | gb = AF004591/gi = 2209342/ug = Mm.9703/len = 432/STRA = for | | | | | | | |
| 4 | 102009_at | Cluster Incl AI835274 UI-M-AQ0-aab-c-12-0-UI s1 Mus musculus cDNA, 3 end/clone = UI-M-AQ0-aab-c-12-0-UI/ | P | 175.6 | P | 322.1 | P | 264 | P |
| | | clone_end = 3/gb = AI835274/gi = 5469487/ug = Mm.100115/len = 460/STRA = rev | | | | | | | |
| 4 | 102056_f_at | Cluster Incl AA839379: vw48g12.r1 Mus musculus cDNA, 5 end/clone = IMAGE-1247110/clone_end = 5/gb = AA839379/ | P | 257.7 | P | 331.1 | P | 190 | P |
| | | gi = 2915474/ug = Mm.10374/len = 500/STRA = for | | | | | | | |
| 4 | 102062_at | Cluster Incl U85614: Mus musculus SRG3 mRNA, complete cds/cds = (60,3362)/gb = U85614/gi = 1816634/ug = Mm 1050/ | P | 234.4 | P | 1162.7 | P | 363.4 | P |
| | | len = 4765/STRA = for | | | | | | | |
| 4 | 102069_at | Cluster Incl S78454 Mus musculus metal response element DNA-binding protein M96 mRNA, complete cds/cds = (0,1121)/ | P | 380.6 | P | 563.3 | P | 356.2 | P |
| | | gb = S78454/gi = 998845/ug = Mm.10563/len = 1517/STRA = for | | | | | | | |
| 4 | 102754_at | Cluster Incl Y17267: Mus musculus mRNA for ubiquitin conjugating enzyme/cds = (258,14795)/gb = Y17267/gi = 3319989/ | P | 253.5 | P | 343.7 | P | 198.4 | P |
| | | ug = Mm 12921/len = 15475/STRA = for | | | | | | | |
| 4 | 102793_at | Cluster Incl D86423 Mus musculus mRNA for HGT keratin, partial cds/cds = (0,163)/gb = D86423/gi = 1944398/ | P | 365.4 | P | 1732.6 | P | 353.4 | P |
| | | ug = Mm.13979/len = 508/STRA = for | | | | | | | |
| 4 | 103067_at | Cluster Incl Z48587: M musculus ral-A mRNA/cds = (0,620)/gb = Z48587/gi = 1107741/ug = Mm 16779/len = 621/STRA = for | P | 420.4 | P | 450.9 | P | 368 | P |
| 4 | 103341_at | Cluster Incl U49350.Mus musculus CTP synthetase mRNA, complete cds/cds = (111,1886)/gb = U49350/gi = 1515356/ | P | 811.3 | P | 1255.5 | P | 908.4 | P |
| | | ug = Mm 1815/len = 2681/STRA = for | | | | | | | |
| 4 | 103344_at | Cluster Incl L16953: Mouse tumor cell dna-j-like protein 1 mRNA, complete cds/cds = (7,1665)/gb = L16953/gi = 473846/ | P | 305.7 | P | 454.5 | P | 315.3 | P |
| | | ug = Mm 18237/len = 1792/STRA = for | | | | | | | |
| 4 | 103413_at | Cluster Incl AI021091 ub01b04 r1 Mus musculus cDNA, 5 end/clone = IMAGE-1365679/clone_end = 5/gb = AI021091/ | P | 149 | P | 296.1 | P | 104.9 | P |
| | | gi = 3235427/ug = Mm 18834/len = 426/STRA = for | | | | | | | |
| 4 | 103415_at | Cluster Incl AI840925: UI-M-AM0-adm-g-07-0-UI.s1 Mus musculus cDNA, 3 end/clone = UI-M-AM0-adm-g-07-0-UI/ | P | 204.2 | P | 228.2 | P | 208.9 | P |
| | | clone_end = 3/gb = AI840925/gi = 5475138/ug = Mm 18847/len = 279/STRA = for | | | | | | | |
| 4 | 103619_at | Cluster Incl AI850017: UI-M-BG0-aib-g-03-0-UI.s1 Mus musculus cDNA, 3 end/clone = UI-M-BG0-aib-g-03-0-UI/ | P | 515.5 | P | 643.4 | P | 381.3 | P |
| | | clone_end = 3/gb = AI850017/gi = 5493923/ug = Mm 20242/len = 428/STRA = rev | | | | | | | |
| 4 | 104317_at | Cluster Incl AW123099: UI-M-BH2 1-apc-e-04-0-UI s1 Mus musculus cDNA, 3 end/clone = UI-M-BH2 1-apc-e-04-0-UI/ | P | 299.8 | P | 367.4 | P | 206 | P |
| | | clone_end = 3/gb = AW123099/gi = 6098629/ug = Mm 22417/len = 610/STRA = for | | | | | | | |
| 4 | 94418_at | Cluster Incl AI839004: UI-M-AN0-acm-c-04-0-UI.s1 Mus musculus cDNA, 3 end/clone = UI-M-AN0-acm-c-04-0-UI/ | P | 357.1 | P | 1256.2 | P | 255.4 | P |
| | | clone_end = 3/gb = AI839004/gi = 5473217/ug = Mm 26171/len = 428/STRA = for | | | | | | | |
| 4 | 94970_at | Cluster Incl AI852314 UI-M-BH0-aje-g-07-0-UI.s1 Mus musculus cDNA, 3 end/clone = UI-M-BH0-aje-g-07-0-UI/ | P | 204.9 | P | 251.6 | P | 192 | P |
| | | clone_end = 3/gb = AI852314/gi = 5496220/ug = Mm 27223/len = 401/STRA = for | | | | | | | |
| 4 | 95002_at | Cluster Incl AI838731 UI-M-AO0-acf-c-07-0-UI.s1 Mus musculus cDNA, 3 end/clone = UI-M-AO0-acf-c-07-0-UI/ | P | 571.7 | P | 704.8 | P | 622.2 | P |
| | | clone_end = 3/gb = AI838731/gi = 5472944/ug = Mm 27362/len = 328/STRA = for | | | | | | | |
| 4 | 95526_at | Cluster Incl AI606243 vo02e01 x1 Mus musculus cDNA, 3 end/clone = IMAGE-1040280/clone_end = 3/gb = AI606243/ | P | 401.4 | P | 444.6 | P | 324.5 | P |
| | | gi = 4615410/ug = Mm 27607/len = 356/STRA = for | | | | | | | |
| 4 | 95619_at | Cluster Incl AI851723 UI-M-BH0-aio-f-07-0-UI s1 Mus musculus cDNA, 3 end/clone = UI-M-BH0-aio-f-07-0-UI/ | P | 107 | P | 188.9 | P | 118 | P |
| | | clone_end = 3/gb = AI851723/gi = 5495624/ug = Mm 28037/len = 391/STRA = for | | | | | | | |
| 4 | 96756_at | Cluster Incl AA693236: vr58d08 r1 Mus musculus cDNA, 5 end/clone = IMAGE-1124847/clone_end = 5/gb = AA693236/ | P | 190.5 | P | 236.1 | P | 126.8 | P |
| | | gi = 2694174/ug = Mm 28983/len = 409/STRA = for | | | | | | | |
| 4 | 97918_at | Cluster Incl AA623587 vn73d05 r1 Mus musculus cDNA, 5 end/clone = IMAGE-1037577/clone_end = 5/gb = AA623587/ | P | 569.4 | P | 786.3 | P | 639.6 | P |
| | | gi = 2527483/ug = Mm.30122/len = 163/STRA = for | | | | | | | |
| 4 | 97954_at | Cluster Incl X59513 Mouse 5 end of TRP1 gene for tyrosinase-related protein-1/cds = (181,183)/gb = X59513/gi = 54913/ | P | 115.5 | P | 307.2 | P | −5.7 | P |
| | | ug = Mm 30438/len = 272/STRA = for | | | | | | | |
| 4 | 98024_at | Cluster Incl X55316 Nuclear transcription factor-Y beta/cds = (209,832)/gb = X55316/gl = 53362/ug = Mm 3259/len = 1234/ | P | 311.3 | P | 584.5 | P | 141.6 | P |
| | | STRA = for | | | | | | | |
| 4 | 98404_at | Cluster Incl X64587 M. musculus mRNA for splicing factor U2AF (65 kD)/cds = UNKNOWN/gb = X64587/gi = 55100/ | P | 1773.8 | P | 2389.8 | P | 1818.1 | P |
| | | ug = Mm 3358/len = 2119/STRA = for | | | | | | | |
| 4 | 98415_at | Cluster Incl AF046060 Mus musculus cytokine receptor related protein 4 (Cytor4) mRNA, complete cds/cds = (54,1382)/ | P | 470.4 | P | 639.7 | P | 433.4 | P |
| | | gb = AF046060/gi = 4105473/ug = Mm.33962/len = 2365/STRA = for | | | | | | | |
| 4 | 98997_f _at | Cluster Incl AF020790 Mus musculus hair keratin acidic 5 (Ha5) mRNA, complete cds/cds = (129,1406)/gb = AF020790/ | P | 826.2 | P | 2557.1 | P | 1080.4 | P |
| | | gi = 4103157/ug = Mm.37952/len = 1737/STRA = rev | | | | | | | |
| 4 | 99452_at | Cluster Incl U49507: Mus musculus B6CBA Lisch7 mRNA, partial cds/cds = (0,1971)/gb = U49507/gi = 1236082/ | P | 293.9 | P | 564.7 | P | 312 | P |
| | | ug = Mm 4067/len = 2059/STRA = for | | | | | | | |
| 4 | 100886_f_at | Cluster Incl AI841415 UI-M-AH0-acx-c-02-0-UI s1 Mus musculus cDNA, 3 end/clone = UI-M-AH0-acx-c-02-0-UI/ | P | 306.5 | P | 404.2 | P | 255 | P |
| | | clone_end = 3/gb = AI841415/gi = 5475628/ug = Mm.5381/len = 303/STRA = for | | | | | | | |
| 4 | 100924_at | Cluster Incl X55123 GATA-binding protein 3/cds = (207,1538)/gb = X55123/gi = 51052/ug = Mm 606/len = 2056/STRA = for | P | 1150.4 | P | 2802.1 | P | 1472.6 | P |
| 4 | 101469_at | Cluster Incl AF009368 Mus musculus neural precursor cell expressed developmentally downregulated Nedd9 (Nedd9) | P | 177.1 | P | 232.6 | P | 192.9 | P |
| | | mRNA, complete cds/cds = (292,2793)/gb = AF009366/gi = 2454522/ug = Mm 8315/len = 2889/STRA = for | | | | | | | |
| 4 | 101929_at | Cluster Incl AI836322 UI-N-AQ0-aag-a-02-0-UI s2 Mus musculus cDNA, 3 end/clone = UI-M-AQ0-aag-a-02-0-UI/ | P | 206.1 | P | 626.8 | P | 427.6 | P |
| | | clone_end = 3/gb = AI836322/gi = 5470530/ug = Mm.939/len = 211/STRA = for | | | | | | | |
| 4 | 102280_at | Cluster Incl AB006758: Mus musculus Pcdh7 mRNA for BH-protocadherin-a, complete cds/cds = (1004,4213)/ | P | 227.9 | P | 344.3 | P | 178.1 | P |
| | | gb = AB006758/gi = 3513311/ug = Mm 11964/len = 5520/STRA = for | | | | | | | |
| 4 | 102863_at | Cluster Incl AI550530: vx21g12.x1 Mus musculus cDNA, 3 end/clone = IMAGE-1265158/clone_end = 3/gb = AI550530/ | P | 310.6 | P | 415.7 | P | 308.6 | P |
| | | gi = 4482893/ug = Mm 1468/len = 234/STRA = for | | | | | | | |
| 4 | 103243_at | Cluster Incl AF083876 Mus musculus XMP (Xmp) mRNA, complete cds/cds = (109,627)/gb = AF083876/gi = 3462841/ | P | 684.7 | P | 1940.8 | P | 1172.8 | P |
| | | ug = Mm 20423/len = 747/STRA = for | | | | | | | |
| 4 | 103501_at | Cluster Incl U02098 Mus musculus Pur-alpha mRNA, complete cds/cds = (518,1483)/gb = U02098/gi = 404649/ug = Mm 2236/ | P | 394.6 | P | 739.3 | P | 521.7 | P |
| | | len = 1609/STRA = for | | | | | | | |
| 4 | 103762_at | Cluster Incl AI853340 UI-M-BH0-air-b-06-0-UI s1 Mus musculus cDNA, 3 end/clone = UI-M-BH0-air-b-06-0-UI/ | P | 430.8 | P | 477 | P | 459.6 | P |
| | | clone_end = 3/gb = AI853340/gi = 5497246/ug = Mm.24632/len = 461/STRA = for | | | | | | | |
| 4 | 104183_at | Cluster Incl AI529005.ud44a03 y1 Mus musculus cDNA, 5 end/clone = IMAGE-1448716/clone_end = 5/gb-AI529005/ | P | 231.3 | P | 302 | P | 221.3 | P |
| | | gi = 4443140/ug = Mm 27380/len = 460/STRA = for | | | | | | | |
| 4 | 104220_at | Cluster Incl AF010133 Mus musculus Smad6 mRNA, complete cds/cds = (0,1487)/gb = AF010133/gi = 2507639/ | P | 294.7 | P | 451.6 | P | 366.9 | P |
| | | ug = Mm.27935/len = 1488/STRA = for | | | | | | | |
| 4 | 92409_at | Cluster Incl D45210: Mouse mRNA for zinc finger protein, complete cds/cds = (590,1813)/gb = D45210/gi = 633989/ | P | 120.2 | P | 169.8 | P | 82.4 | P |
| | | ug = Mm.4106/len = 2849/STRA = for | | | | | | | |
| 4 | 92460_at | Cluster Incl AB022316: Mus musculus gene for semaphorin w/cds = (78,2411)/gb = AB022316/gi = 4589501/ug = Mm.42035/ | P | 911.5 | P | 1765.6 | P | 1033.2 | P |
| | | len = 4024/STRA = for | | | | | | | |
| 4 | 92888_s_at | Cluster Incl U34973: Mus musculus protein tyrosine phosphatase-like mRNA, unspliced c-terminal product and spliced c- | P | 370 | P | 446 | P | 367.4 | P |
| | | terminal and STYX, complete cds/cds = (255,872)/gb = U34973/gi = 1063624/ug = Mm 4755/len = 1254/STRA = for | | | | | | | |
| 4 | 92893_at | Cluster Incl D90173 Mouse mRNA for NFI-B protein, clone NFI-B2/cds = (111,1709)/gb = D90173/gi = 220518/ug = Mm 4771/ | P | 243.3 | P | 506.6 | P | 275.4 | P |
| | | len = 3296/STRA = for | | | | | | | |
| 4 | 93246_at | Cluster Incl AW260482: um80e10.x1 Mus musculus cDNA, 3 end/clone = IMAGE-2317674/clone_end = 3/gb = AW260482/ | P | 297.4 | P | 677.1 | P | 479.1 | P |
| | | gi = 6633463/ug = Mm 6043/len = 635/STRA = rev | | | | | | | |
| 4 | 93439_f_at | Cluster Incl AA260005 va87f08.r1 Mus musculus cDNA, 5 end/clone = IMAGE-746435/clone_end = 5/gb = AA260005/ | P | 306.2 | P | 421.8 | P | 265.8 | P |
| | | gi = 1896491/ug = Mm.6617/len = 271/STRA = for | | | | | | | |
| 4 | 93667_at | Cluster Incl AW120511 UI-M-BH1-ana-a-04-0-UI s2 Mus musculus cDNA, 3 end/clone = UI-M-BH1-ana-a-04-0-UI/ | P | 121.8 | P | 344.5 | P | 41.8 | P |
| | | clone_end = 3/gb = AW120511/gi = 6095844/ug = Mm.80527/len = 454/STRA = for | | | | | | | |
| 4 | 97767_at | Cluster Incl X75649 M musculus mRNA for mouse hair acidic 2/cds = (28,1251)/gb = X75649/gi = 498977/ug = Mm 4161/ | P | 846.2 | P | 1835.4 | P | 1468.8 | P |
| | | len = 1591/STRA = for | | | | | | | |
| 4 | 99328_at | Cluster Incl U79738 Mus musculus homeodomain protein Dix-3 mRNA, parital cds/cds = (0,695)/gb = U79738/gi = 1718494/ | P | 633.3 | P | 1253 | P | 557.8 | P |
| | | ug = Mm.5194/len = 2221/STRA = for | | | | | | | |
| 4 | 100323_at | Cluster Incl Z23077 S-adenosylmethionine decarboxylase 3/cds = (0,1004)/gb = Z23077/gi = 396140/ug = Mm 87749/ | P | 417 | P | 988.6 | P | 559.6 | P |
| | | len = 1005/STRA = for | | | | | | | |
| 4 | 100324_g_at | Cluster Incl Z23077 S-adenosylmethionine decarboxylase 3/cds = (0,1004)/gb = Z23077/gi = 396140/ug = Mm 87749/ | P | 122.7 | P | 200.2 | P | 112.2 | P |
| | | len = 1005/STRA = for | | | | | | | |
| 4 | 101254_at | Cluster Incl L32751 Lipopolysacchande response/cds = (25,675)/gb = L32751/gi = 727168/ug = Mm 7521/len = 994/STRA = for | P | 4334.5 | P | 6277.7 | P | 4162 | P |
| 4 | 102099_f_at | Cluster Incl AI843637.UI-M-AO1-aen-g-04-0-UI s1 Mus musculus cDNA, 3 end/clone = UI-M-AO1-aen-g-04-0-UI/ | P | 539.3 | P | 853.5 | P | 550.6 | P |
| | | clone_end = 3/gb = AI843637/gi = 5477850/ug = Mm 42435/len = 238/STRA = rev | | | | | | | |
| 4 | 102103_f_at | Cluster Incl AI482454 vg40d04 x1 Mus musculus cDNA, 3 end/clone = IMAGE-863815/clone_end = 3/gb = AI482454/ | P | 1527.8 | P | 2263.6 | P | 1907.3 | P |
| | | gi = 4375680/ug = Mm 42484/len = 454/STRA = rev | | | | | | | |
| 4 | 92614_at | Cluster Incl M60523.Inhibitor of DNA binding 3/cds = (57,416)/gb = M60523/gi = 193872/ug = Mm 110/len = 969/STRA = for | P | 387.2 | P | 498.1 | P | 363 | P |
| 4 | 93348_at | Cluster Incl AA760359 vv76a05 r1 Mus musculus cDNA, 5 end/clone = IMAGE-1228304/clone_end = 5/gb = AA760359/ | P | 149.6 | P | 262.3 | P | 183 | P |
| | | gi = 2808153/ug = Mm 18803/len = 373/STRA = for | | | | | | | |
| 4 | 97814_at | Cluster Incl x75650 M musculus mRNA for mouse hair acidic 3/cds = (0,971)/gb = X75650/gi = 498975/ug = Mm.28178/ | P | 445.3 | P | 1322 | P | 464.2 | P |
| | | len = 1304/STRA = for | | | | | | | |
| 4 | 95649_at | Cluster Incl AI844440 UI-M-AJ1-ahe-a-02-0-UI s1 Mus musculus cDNA, 3 end/clone = UI-M-AJ1-ahe-a-02-0-UI/ | P | 445.1 | P | 577.6 | P | 438.5 | A |
| | | clone_end = 3/gb = AI844440/gi = 5488346/ug = Mm 29503/len = 301/STRA = for | | | | | | | |
| 4 | 95677_at | Cluster Incl AA881621 vx20h04 r1 Mus musculus cDNA, 5 end/clone = IMAGE-1265047/clone_end = 5/gb = AA881621/ | P | 203.7 | P | 375.9 | P | 114.4 | M |
| | | gi = 2990931/ug = Mm 29606/len = 507/STRA = for | | | | | | | |
| 4 | 99644_at | Cluster Incl AI845855-UI-M-AO1-aeg-e-11-0-UI s1 Mus musculus cDNA, 3 end/clone = UI-M-AO1-aeg-e-11-0-UI/ | P | 443.8 | P | 780.5 | P | 511.6 | P |
| | | clone_end = 3/gb = AI845855/gi = 5489761/ug = Mm 43636/len = 407/STRA = for | | | | | | | |
| 4 | 103558_at | Cluster Incl M27734 Mouse acedic keratin type I mRNA, complete cds/cds = (69,1319)/gb = M27734/gi = 198585/ | P | 312 | P | 1514.2 | P | 357.5 | P |
| | | ug = Mm.19109/len = 1585/STRA = for | | | | | | | |
| 4 | 103803_at | Cluster Incl AB013849 Mus musculus mRNA for peptidylarginine deiminase type III, complete cds/cds = (42,2038)/ | P | 198.7 | P | 353 | P | 148.6 | A |
| | | gb = AB013849/gi = 3645569/ug = Mm.20851/len = 3068/STRA = for | | | | | | | |
| 4 | 104124_at | Cluster Incl AW123135 UI-M-BH2.1-apd-a-07-0-UI s1 Mus musculus cDNA, 3 end/clone = UI-M-BH2-apd-a-07-0-UI/ | P | 143.3 | P | 248.8 | P | 89 | M |
| | | clone_end = 3/gb = AW123135/gi = 6098665/ug = Mm 22028/len = 503/STRA = for | | | | | | | |
| 4 | 96138_at | Cluster Incl AA682129: vu13b07.r1 Mus musculus cDNA, 5 end/clone = IMAGE-1180501/clone_end = 5/gb = AA682129/ | P | 312.7 | P | 561.9 | P | 280.7 | P |
| | | gi = 2663161/ug = Mm 28173/len = 611/STRA = for | | | | | | | |
| 4 | 96822_at | Cluster Incl AA734426 vr90e10 r1 Mus musculus cDNA, 5 end/clone = IMAGE-1136010/clone_end = 5/gb = AA734428/ | P | 450 | P | 565.1 | P | 465.5 | P |
| | | gi = 2756095/ug = Mm 29383/len = 409/STRA = rev | | | | | | | |
| 4 | 97983_s_at | Cluster Incl D45903. Mus musculus mRNA for unc-18 homologue, complete cds/cds = (35,1819)/gb = D45903/gi = 1944321/ | P | 164.6 | P | 344.7 | P | 188.1 | P |
| | | ug = Mm.3129/len = 3412/STRA = for | | | | | | | |
| 4 | 99502_at | Cluster Incl U80078 Mus musculus transcription factor BFCOL1 mRNA, complete cds/cds = (86,2395)/gb = U80078/ | P | 530.4 | P | 1058.2 | P | 566.8 | M |
| | | gi = 1724123/ug = Mm 417/len = 2426/STRA = for | | | | | | | |
| 4 | 100895_at | Cluster Incl AW049497: UI-M-BH1-anf-a-01-0-UI s1 Mus musculus cDNA, 3 end/clone = UI-M-BH1-anf-a-01-0-UI/ | P | 41.4 | P | 161.5 | P | 43.9 | P |
| | | clone_end = 3/gb = AW049497/gi = 5910026/ug = Mm 567/len = 291/STRA = for | | | | | | | |
| 4 | 101376_at | Cluster Incl D86419 Mus musculus mRNA for glycine tyrosine-rich hair keratin protein, complete cds/cds = (2,235)/ | P | 363.3 | P | 1186.6 | P | 435.1 | P |
| | | gb = D86419/gi = 1944392/ug = Mm 6973/len = 520/STRA = for | | | | | | | |
| 4 | 103791_at | Cluster Incl AW048763 UI-M-BH1-amy-h-02-0-UI s1 Mus musculus cDNA, 3 end/clone = UI-M-BH1-amy-h-02-0-UI/ | P | 151.2 | P | 193 | P | 51.2 | P |
| | | clone_end = 3/gb = AW048763/gi = 5909292/ug = Mm 24958/len = 589/STRA = for | | | | | | | |
| 4 | 103998_at | Cluster Incl AW122699 UI-M-BH2 2-aot-b-09-0-UI s1 Mus musculus cDNA, 3 end/clone = UI-M-BH2 2-aot-b-09-0-UI/ | P | 94.2 | P | 135.1 | P | 67.3 | P |
| | | clone_end = 3/gb = AW122699/gi = 6098229/ug = Mm 26532/len = 435/STRA = for | | | | | | | |
| 4 | 104242_f_at | Cluster Incl AI835622 UI-M-AI0-aap-f-12-0-UI s1 Mus musculus cDNA, 3 end/clone = UI-M-AI0-aap-f-12-0-UI/clone_end = 3/ | P | 971.3 | P | 1411.3 | P | 1260.2 | P |
| | | gb = AI835622/gi = 5469835/ug = Mm.2821/len = 344/STRA = for | | | | | | | |
| 4 | 104643_at | Cluster Incl AI850848 UI-M-BH0-ajt-b-11-0-UI s1 Mus musculus cDNA, 3 end/clone = UI-M-BH0-ajt-b-11-0-UI/clone_end = 3/ | P | 261.5 | P | 440.9 | P | 198.4 | A |
| | | gb = AI850846/gi = 5494752/ug = Mm 31267/len = 341/STRA = for | | | | | | | |
| 4 | 104688_at | Cluster Incl Y09010 M musculus mRNA for serine/threonine kinase/cds = (138,2621)/gb = Y09010/gi = 1654011/ | P | 112.6 | P | 224.9 | P | 67.8 | M |
| | | ug = Mm 3313/len = 2732/STRA = for | | | | | | | |
| 4 | 104696_at | Cluster Incl AJ009840 Mus musculus cathepsin E gene, exon 1, partial/cds = (0,1193)/gb = AJ009840/gi = 3392946/ | P | 107.9 | P | 237.5 | P | 133 | P |
| | | ug = Mm 33671/len = 1194/STRA = for | | | | | | | |
| 4 | 93720_at | Cluster Incl AB005623: Mus musculus mRNA for 1-acyl-sn-glycerol-3-phosphate acyltransferase, complete cds/ | P | 147.8 | P | 274.9 | P | 102 | M |
| | | cds = (318,1175)/gb = AB005623/gi = 2457309/ug = Mm.8684/len = 1256/STRA = for | | | | | | | |
| 4 | 93935_at | Cluster Incl AF036332: Mus musculus nemo-like kinase (NIk) mRNA, complete cds/cds = (322,1869)/gb = AF036332/ | P | 76.4 | P | 113.7 | P | 79.7 | P |
| | | gi = 2828007/ug = Mm.9001/len = 2603/STRA = for | | | | | | | |
| 4 | 98340_at | Cluster Incl AA407332: EST00652 Mus musculus cDNA, 5 end/clone = C0001A09/clone_end = 5/gb = AA407332/ | P | 23 | P | 136.7 | P | 70.2 | P |
| | | gi = 2065567/ug = Mm 43174/len = 365/STRA = rev | | | | | | | |
| 4 | 100381_at | Cluster Incl M12347: Actin, alpha 1, skeletal muscle/cds = (70,1203)/gb = M1234/gi = 191572/ug = Mm.89137/len = 1446/ | P | 2656.5 | P | 4920.4 | P | 2682.5 | A |
| | | STRA = for | | | | | | | |
| 4 | AFFX- | E Coli BioB 3 prime | P | 405.9 | P | 490.7 | P | 361.2 | P |
| | BioB-_at | | | | | | | | |
| 4 | 93046_at | Cluster Incl AW045233 UI-M-BH1-alh-f-10-0-UI s1 Mus musculus cDNA, 3 end/clone = UI-M-BH1-alh-f-10-0-UI/ | P | 158.5 | P | 243.8 | P | 105.8 | A |
| | | clone_end = 3/gb = AW045233/gi = 5905762/ug = Mm 1521/len = 412/STRA = for | | | | | | | |
| 4 | 93971_f_at | Cluster Incl AI838669 UI-M-AO0-aca-e-04-0-UI s1 Mus musculus cDNA, 3 end/clone = UI-M-AO0-aca-e-04-0-UI/ | P | 351.3 | P | 517.7 | P | 373.5 | P |
| | | clone_end = 3/gb = AI838669/gi = 5472882/ug = Mm 21667/len = 452/STRA = for | | | | | | | |
| 4 | 94064_at | Cluster Incl U05343: Mus musculus Balb/c zinc finger protein PZF (Pzf) mRNA, complete cds/cds = (137,1504)/gb = U05343/ | P | 194.1 | A | 512.5 | P | 152.3 | P |
| | | gi = 453375/ug = Mm 22066/len = 1514/STRA = for | | | | | | | |
| 4 | 94804_at | Cluster Incl L27453: Pre B-cell leukemia transcription factor 1/cds = (44,1087)/gb = L27453/gi = 456108/ug = Mm 22675/ | P | 78.2 | M | 232.6 | P | 166.3 | P |
| | | len = 1634/STRA = for | | | | | | | |
| 4 | 94493_at | Cluster Incl AF095905: Mus musculus CPETR2 (Cpetr2) mRNA, complete cds/cds = (55,714)/gb = AF095905/gi = 4262394/ | M | 128.9 | P | 194.8 | P | 143.7 | P |
| | | ug = Mm 28921/len = 1090/STRA = for | | | | | | | |
| 4 | 95658_at | Cluster Incl AW061085 UI-M-BH1-anp-c-04-0-UI s1 Mus musculus cDNA, 3 end/clone = UI-M-BH1-anp-c-04-0-UI/ | P | 71.7 | P | 159.8 | P | 49.8 | A |
| | | clone_end = 3/gb = AW061085/gi = 6008836/ug = Mm 29542/len = 464/STRA = for | | | | | | | |
| 4 | 96918_at | Cluster Incl AI790931 uk29g y1 Mus musculus cDNA, 5 end/clone = IMAGE-1970450/clone_end = 5/gb = AI790931/ | P | 314.7 | P | 981.9 | P | 389.9 | P |
| | | gi = 5338647/ug = Mm 30154/len = 660/STRA = for | | | | | | | |
| 4 | 102415_r_at | Cluster Incl U28423 Mus musculus protein kinease inhibitor p58 mRNA, complete cds/cds = (100,1614)/gb = U28423/ | P | 136.5 | A | 333.7 | P | 112.7 | M |
| | | gi = 1353271/ug = Mm 12616/len = 1615/STRA = for | | | | | | | |
| 4 | 104096_at | Cluster Incl Y16386: Mus musculus mRNA for origin recognition complex subunit 4/cds = (0,1301)/gb = Y16386/gi = 3288543/ | P | 149.6 | P | 218.2 | P | 131.4 | P |
| | | ug = Mm 21834/len = 1302/STRA = for | | | | | | | |
| 4 | 104626_at | Cluster Incl AI787137 ui85c11.y1 Mus musculus cDNA, 5 end/clone = IMAGE-1889204/clone_end = 5/gb = AI787137/ | P | 169.6 | P | 173.6 | P | 150.2 | M |
| | | gi = 5334853/ug = Mm 24277/len = 297/STRA = for | | | | | | | |
| 4 | 96177_at | Cluster Incl AI854590 UI-M-BH0-ake-b-04-0-UI s1 Mus musculus cDNA, 3 end/clone = UI-M-BH0-ake-b-04-0-UI/ | P | 98.9 | P | 220.9 | A | 21.5 | P |
| | | clone_end = 3/gb = AI854590/gi = 549896/ug = Mm.28412/len = 435/STRA = for | | | | | | | |
| 4 | 98407_at | Cluster Incl U07602 Ephnn B1/cds = (1643,2680)/gb = U07602/gi = 2182217/ug = Mm 3374/len = 2692/STRA = rev | P | 196.3 | P | 432 | P | 239.2 | A |
| 4 | 102308_at | Cluster Incl AF045582: Mus musculus tubby protein 3 (Tulp3) mRNA, complete cds/cds = (26,1408)/gb = AF045582/ | A | 422 | P | 547.1 | P | 385.9 | A |
| | | gi = 3372490/ug = Mm 12887/len = 1508/STRA = for | | | | | | | |
| 4 | 103552_at | Cluster Incl AW046470 UI-M-BH1-akp-h-12-0-UI s1 Mus musculus cDNA, 3 end/clone = UI-M-BH1-akp-h-12-0-UI/ | M | 120.4 | P | 200.7 | P | 67.3 | A |
| | | clone_end = 3/gb = AW046470/gi = 5906999/ug = Mm 23822/len = 449/STRA = rev | | | | | | | |
| 4 | 95801_s_at | Cluster Incl L36316: Zinc finger protein 63/cds = (0,556)/gb = L36316/gi = 538414/ug = Mm 87838/len = 858/STRA = for | A | 135.9 | P | 187.5 | P | 80.1 | A |
| 4 | 93981_at | Cluster Incl J03520 Plasminogen acitvator, tissue/cds = (93,1772)/gb = J03520/gi = 202109/ug = Mm.21702/len = 2519/ | A | 234.7 | A | 649.6 | P | 126.7 | P |
| | | STRA = for | | | | | | | |
| 4 | 94222_at | Cluster Incl AI838737 UI-M-AO0-acf-d-01-0-UI.s1 Mus musculus cDNA, 3 end/clone = UI-M-AO0-acf-d-01-0-UI/ | M | 531.2 | P | 644.5 | P | 508.4 | A |
| | | clone_end = 3/gb = AI838737/gi = 5472950/ug = Mm 22248/len = 399/STRA = for | | | | | | | |
| 4 | 94916_at | Cluster Incl AW122260 UI-M-BH2 2-aov-g-11-0-UI s1 Mus musculus cDNA, 3 end/clone = UI-M-BH2 2-aov-g-11-0-UI/ | P | 293.2 | P | 473.9 | P | 256.6 | M |
| | | clone_end = 3/gb = AW122260/gi = 6097729/ug = Mm 24155/len = 161/STRA = for | | | | | | | |
| 4 | 96294_s_at | Cluster Incl AI854235 UI-M-BH0-ajz-g-07-0-UI s1 Mus musculus cDNA, 3 end/clone = UI-M-BH0-ajz-g-07-0-UI/ | P | 195.4 | P | 237.2 | P | 90 | A |
| | | clone_end = 3/gb = AI854235/gi = 5498141/ug = Mm 29898/len = 519/STRA = for | | | | | | | |
| 4 | 100607_at | Cluster Incl AF026124: Mus musculus schwannoma-associated protein (SAM9) mRNA, complete cds/cds = (417,1883)/ | P | 509.6 | P | 607.8 | A | 248.4 | P |
| | | gb = AF026124/gi = 2565395/ug = Mm 6483/len = 2248/STRA = for | | | | | | | |
| 4 | 102826_at | Cluster Incl J05663: Androgen regulated vas deferens protein/cds = (20,970)/gb = J05663/gi = 199897/ug = Mm.14460/ | A | 101.2 | P | 128.7 | P | 92 | A |
| | | len = 1225/STRA = for | | | | | | | |
| 4 | 103006_at | Cluster Incl AB012276 Mus musculus MRNA for ATFx, partial cds/cds = (0,254)/gb = AB012276/gi = 2988372/ug = Mm 1566/ | P | 323.4 | P | 421.9 | P | 288.6 | A |
| | | len = 850/STRA = for | | | | | | | |
| 4 | 103565_at | Cluster Incl AI843358 UI-M-AQ1-adz-d-04-0-UI s1 Mus musculus cDNA, 3 end/clone = UI-M-AQ1-adz-d-04-0-UI/ | P | 440.4 | P | 678.9 | P | 286.9 | A |
| | | clone_end = 3/gb = AI84358/gi = 5477571/ug = Mm.1915/len = 444/STRA = for | | | | | | | |
| 4 | 104346_at | Cluster Incl AI841914 UI-M-AO0-acd-f-08-0-UI.s1 Mus musculus cDNA, 3 end/clone = UI-M-AO0-acd-f-08-0-UI/ | P | 214.7 | P | 465.8 | P | 217.2 | P |
| | | clone_end = 3/gb = AI841914/gi = 5476127/ug = Mm.22537/len = 421/STRA = for | | | | | | | |
| 4 | 95596_at | Cluster Incl AW122957 UI-M-BH2 1-aoz-e-12-0-UI.s2 Mus musculus cDNA, 3 end/clone = UI-M-BH2 1-aoz-e-12-0-UI/ | P | 163.9 | P | 220.1 | A | 147.6 | P |
| | | clone_end = 3/gb = AW122957/gi = 6098487/ug = Mm.27918/len = 443/STRA = for | | | | | | | |
| 4 | 96735_at | Cluster Incl AW049732 UI-M-BH1-ani-g-05-0-UI.s1 Mus musculus cDNA, 3 end/clone = UI-M-BH1-ani-g-05-0-UI/ | P | 192.4 | P | 527.6 | P | 288.4 | A |
| | | clone_end = 3/gb = AW049732/gi = 5910261/ug = Mm.28896/len = 372/STRA = for | | | | | | | |
| 4 | 98998_r_at | Cluster Incl AF020790 Mus musculus hair keratin acidic 5 (Ha5) mRNA, complete cds/cds = (129,1406)/gb = AF020790/ | P | 113.9 | P | 620 | P | 148.9 | A |
| | | gi = 4103157/ug = Mm.37952/len = 1737/STRA = rev | | | | | | | |
| 4 | 102642_at | Cluster Incl L33415 Natural resistance-associated macrophage protein 2/cds = (60,1766)/gb = L33415/gi = 755040/ | P | 967.4 | P | 1654.6 | P | 630.4 | A |
| | | ug = Mm.1304/len = 2972/STRA = for | | | | | | | |
| 4 | 103257_at | Cluster Incl AA690483: vu52f06.r1 Mus musculus cDNA, 5 end/clone = IMAGE-1195043/clone_end = 5/gb = AA690483/ | P | 145.6 | P | 230 | P | 113.9 | M |
| | | gl = 2691419/ug = Mm.2073/len = 302/STRA = for | | | | | | | |
| 4 | 103965_at | Cluster Incl AI837032 UI-M-AP0-abj-d-02-0-UI s2 Mus musculus cDNA, 3 end/clone = UI-M-AP0-abj-d-02-0-UI/ | P | 102.3 | P | 557.4 | P | 97.6 | A |
| | | clone_end = 3/gb = AI837032/gi = 5471245/ug = Mm.25640/len = 289/STRA = for | | | | | | | |
| 4 | 98314_g_at | Cluster Incl AB025413.Mus musculus mRNA for Ten-m4, complete cds/cds = (187,8502)/gb = AB025413/gi = 4760781/ | P | 120.6 | M | 333.2 | P | 60.1 | A |
| | | ug = Mm.42204/len = 8585/STRA = for | | | | | | | |
| 4 | 100673_f_at | Cluster Incl U10091 Killer cell lectin-like receptor, subfamily A, member 5/cds = (108,908)/gb = U10091/gi = 533489/ | P | 37.6 | P | 100.4 | P | 29.3 | A |
| | | ug = Mm.14111/len = 1157/STRA = for | | | | | | | |
| 4 | 98881_at | Cluster Incl AI853675: UI-M-BH0-ajq-g-08-0-UI.s1 Mus musculus cDNA, 3 end/clone = UI-M-BH0-ajq-g-08-0-UI/ | A | 173.7 | P | 196.8 | P | 102.1 | P |
| | | clone_end = 3/gb = AI853675/gi = 5497621/ug = Mm.28525/len = 528/STRA = for | | | | | | | |
| 4 | 101006_at | Cluster Incl M35797: T-complex protein 1, related sequence 1/cds = (0,852)/gb = M35797/gi = 975268/ug = Mm.6797/ | P | 454.2 | P | 803.3 | P | 275.6 | A |
| | | len = 1692/STRA = for | | | | | | | |
| 4 | 102989_at | Cluster Incl U07808: Metallothionein 4/cds = (21,209)/gb = U07808/gi = 466268/ug = Mm 1536/len = 372/STRA = for | P | 133.5 | P | 380.1 | P | 141.9 | A |
| 4 | 103805_at | Cluster Incl AF092840: Mus musculus nibnn mRNA, complete cds/cds = (90,2345)/gb = AF092840/gi = 3676834/ | | 141.7 | P | 199.9 | P | 121.6 | M |
| | | ug = Mm 20866/len = 2473/STRA = for | | | | | | | |
| 4 | 104364_at | Cluster Incl AF039840 Mus musculus mitogen-activated protein kinase-activated protein kinase MRNA, complete cds/ | P | 124.8 | P | 228.1 | P | −42.4 | A |
| | | cds = (706,2127)/gb = AF039840/gi = 2911812/ug = Mm 22612/len = 2243/STRA = for | | | | | | | |
| 4 | 95556_at | Cluster Incl AI841364: UI-M-AM0-adv-f-01-0-UI s1 Mus musculus cDNA, 3 end/clone = UI-M-AM0-adv-f-01-0-UI/ | P | 96.4 | P | 195.4 | P | −13.7 | A |
| | | clone_end = 3/gb = AI841364/gi = 5475577/ug = Mm 27754/len = 389/STRA = for | | | | | | | |
| 4 | 97343_at | Cluster Incl AI852735 UI-M-BH0-aji-h-03-0-UI s1 Mus musculus cDNA, 3 end/clone = UI-M-BH0-aji-h-03-0-UI/clone_end = 3/ | P | 267.9 | P | 904.7 | A | 539.2 | A |
| | | gb = AI852735/gi = 5496641/ug = Mm 29604/len = 292/STRA = rev | | | | | | | |
| 4 | 98429_at | Cluster Incl AB009653: Mus musculus mRNA for lysophospholipase II, complete cds/cds = (105,800)/gb = AB009653/ | P | 506 | P | 1247.6 | P | 725.4 | P |
| | | gi = 4589452/ug = Mm 34302/len = 1593/STRA = for | | | | | | | |
| 4 | 98488_at | Cluster Incl AJ223361: Mus musculus mRNA for myosin heavy chain 28, partial/cds = (0,93)/gb = AJ223361/gi = 4582681/ | P | 53.1 | P | 141.9 | P | 85.8 | A |
| | | ug = Mm 35531/len = 199/STRA = for | | | | | | | |
| 4 | 101869_s_at | Cluster Incl J00413 Hemoglobin, beta adult major chain/cds = (0,443)/gb = J00413/gi = 193793/ug = Mm 87778/len = 444/ | P | 2339 | P | 5972.7 | P | 1335.8 | A |
| | | STRA = for | | | | | | | |
| 4 | 103449_at | Cluster Incl AA795923 vu05110 r1 Mus musculus cDNA, 5 end/clone = IMAGE-1179787/clone_end = 5/gb = AA795923/ | P | 387.1 | P | 445.5 | A | 336.5 | P |
| | | gi = 2858878/ug = Mm 21577/len = 558/STRA = for | | | | | | | |
| 4 | 104157_at | Cluster Incl AW125643: UI-M-BH2.2.aqi-a-09-0-UI s1 Mus musculus cDNA, 3 end/clone = UI-M-BH2 2-aqi-a-09-0-UI/ | P | 83.2 | P | 143.4 | P | −14.2 | A |
| | | clone_end = 3/gb = AW125643/gi = 6101173/ug = Mm 27090/len = 462/STRA = rev | | | | | | | |
| 4 | 93901_at | Cluster Incl U78315 Pale ear/cds = (139,2250)/gb = U78315/gi = 2190591/ug = Mm 89325/len = 2687/STRA = for | A | 123.3 | P | 280.1 | P | 168.4 | A |
| 4 | 94781_at | Cluster Incl V00714: Hemoglobin alpha, adult chain 1/cds = (33,461)/gb = V00714/gi = 49899/ug = Mm 16820/len = 564/ | P | 2311.5 | P | 4939.6 | P | 1861.4 | A |
| | | STRA = for | | | | | | | |
| 4 | 98330_at | Cluster Incl D70849: Zinc finger protein of the crebellum 3/cds = (538,1938)/gb = D70849/gi = 1345414/ug = Mm 4265/ | A | 147.1 | A | 196.9 | P | 181.9 | M |
| | | len = 3509/STRA = for | | | | | | | |
| 4 | 95858_at | Cluster Incl C76988 C76988 Mus musculus cDNA, 3 end/clone = J0023H02/clone_end = 3/gb = C76988/gi = 2517318/ | A | 73.5 | A | 217 | P | 152.5 | A |
| | | ug = Mm 24929/len = 594/STRA = rev | | | | | | | |
| 4 | 93556_at | Cluster Incl X99143: M musculus mRNA for hair keratin, mHb6/cds = (0,1370)/gb = X99143/gi = 1903237/ug = Mm 20285/ | P | 358.5 | P | 800.1 | P | 477.6 | A |
| | | len = 1808/STRA = for | | | | | | | |
| 4 | 94828_at | Cluster Incl AF004927 Opioid receptor, sigma 1/cds = (61,732)/gb = AF004927/gi = 3493173/ug = Mm 22745/len = 1618/ | A | 320 | P | 539.9 | A | 454.6 | A |
| | | STRA = for | | | | | | | |
| 4 | 103833_at | Cluster Incl AF077659 Mus musculus homeodomain-interacting protein kinase 2 mRNA, complete cds/cds = (157,3726)/ | P | 154.9 | A | 301.1 | P | 54.8 | M |
| | | gb = AF077659/gi = 3702957/ug = Mm 20934/len = 3964/STRA = for | | | | | | | |
| 4 | 98430_at | Cluster Incl X85169 Surfeit gene 5/cds = (188,610)/gb = X85169/gi = 1150511/ug = Mm 3434/len = 3424/STRA = for | P | 39.5 | P | 413.3 | M | −102 | A |
| 4 | 99521_at | Cluster Incl AB020239 Mus musculus mRNA for adenytate kinase 3, complete cds/cds = (82,753)/gb = AB020239/ | P | 105.3 | P | 130.5 | A | 91.4 | A |
| | | gi = 4760605/ug = Mm 42040/len = 916/STRA = for | | | | | | | |
| 4 | 101863_at | Cluster Incl C78246 C78246 Mus musculus cDNA, 3 end/clone = J0044H11/clone_end = 3/gb = C78246/gi = 2518576/ | A | 79.2 | P | 130 | P | 89.9 | P |
| | | ug = Mm 87617/len = 546/STRA = rev | | | | | | | |
| 4 | 102732_at | Cluster Incl AI849746: UI-M-AL1-ahn-d-09-0-UI s1 Mus musculus cDNA, 3 end/clone = UI-M-AL1-ahn-d-09-0-UI/ | A | 167.7 | A | 209.1 | P | 147.1 | A |
| | | clone_end = 3/gb = AI849746/gi = 5493652/ug = Mm 14458/len = 319/STRA = for | | | | | | | |
| 4 | 103447_at | Cluster Incl AI835989.UI-M-AJ0-abb-a-04-0-UI s1 Mus musculus cDNA, 3 end/clone = UI-M-AJ0-abb-a-04-0-UI/ | A | 281.7 | P | 299.6 | P | 55.4 | M |
| | | clone_end = 3/gb = AI835989/gi = 5470242/ug = Mm 21557/len = 316/STRA = for | | | | | | | |
| 4 | 93953_at | Cluster Incl D89871 Mus musculus mRNA for serine protease, complete cds/cds = (244,2529)/gb = D89871/gi = 2696702/ | P | 167.8 | P | 320.6 | P | 151.5 | A |
| | | ug = Mm 9431/len = 2614/STRA = for | | | | | | | |
| 4 | 94712_at | Cluster Incl U73620 Vascular endothelial growth factor C/cds = (153,1400)/gb = U73620/gi = 1778356/ug = Mm 1402/ | P | 37.1 | P | 47.3 | P | 37.7 | P |
| | | len = 1818/STRA = for | | | | | | | |
| 4 | 99681_at | Cluster Incl T25554: EST00587 Mus musculus cDNA, 3 end/clone = B0001C09/clone_end = 3/gb = T25554/gi = 562875/ | A | 120.9 | P | 144.5 | A | 128.4 | A |
| | | ug = Mm 23757/len = 275/STRA = rev | | | | | | | |
| 4 | 93022_i_at | Cluster Incl M32459: Mouse histone H3 (H3 2-221) gene, complete cds/cds = (21,431)/gb = M32459/gi = 193855/ | P | 45.5 | P | 87.1 | P | 7.6 | A |
| | | ug = Mm 14775/len = 595/STRA = for | | | | | | | |
| 4 | 93594_r_at | Cluster Incl U87948 Epithelial membrane protein 3/cds = (201,692)/gb = U87948/gi = 2358253/ug = Mm 20829/len = 695/ | P | 495.5 | P | 538.4 | P | 528.3 | A |
| | | STRA = for | | | | | | | |
| 4 | 99080_at | Cluster Incl AI853930 UI-M-BH0-aju-g-05-0-UI s1 Mus musculus cDNA, 3 end/clone = UI-M-BH0-aju-g-05-0-UI/ | A | 309.9 | P | 379.8 | P | 186.9 | A |
| | | clone_end = 3/gb = AI853930/gi = 5497836/ug = Mm 3886/len = 442/STRA = for | | | | | | | |
| 4 | 103595_at | Cluster Incl AW012359 uo03h08 y1 Mus musculus cDNA, 5 end/clone = IMAGE-2582367/clone_end = 5/gb = AW012359/ | A | 197.4 | A | 343 | P | 223 | P |
| | | gi = 5861137/ug = Mm 19974/len = 537/STRA = for | | | | | | | |
| 4 | 104767_f_at | Cluster Incl AW060921.UI-M-BH1-anv-c-12-0-UI s1 Mus musculus cDNA, 3 end/clone = UI-M-BH1-anv-c-12-0-UI/ | P | 251.7 | P | 401.3 | M | 276.9 | A |
| | | clone_end = 3/gb = AW060921/gi = 6008672/ug = Mm 24875/len = 438/STRA = for | | | | | | | |
| 4 | 96775_at | Cluster Incl X56690: Chromobox homolog (Drosophila HP1 beta)/cds = (0,557)/gb = X56690/gi = 53162/ug = Mm.29055/ | P | 189.4 | A | 306.5 | P | 206.6 | A |
| | | len = 558/STRA = for | | | | | | | |
| 4 | 97997_at | Cluster Inci U88566 Mus musculus secreted frizzled related protein sFRP-1 (Sfrp1) mRNA, complete cds/cds = (256,1200)/ | P | 120.6 | A | 249.9 | P | 117.6 | A |
| | | gb = U88566/gi = 1946340/ug = Mm 3171/len = 2659/STRA = for | | | | | | | |
| 4 | 102192_r_at | Cluster Incl AF068246.Mus musculus SA protein mRNA, complete cds/cds = (274,2010)/gb = AF068246/gi = 3928675/ | P | 21.6 | P | 46.6 | P | 20.5 | A |
| | | ug = Mm 9821/len = 2544/STRA = rev | | | | | | | |
| 4 | 102652_at | Cluster Incl X56959 POU domain, class 3, transcription factor 1/cds = (35,1384)/gb = X56959/gi = 49681/ug = Mm 1330/ | A | 267.5 | P | 548.8 | P | 222.3 | A |
| | | len = 1516/STRA = for | | | | | | | |
| 4 | 103225_at | Cluster Incl AW018420: ul95h03 x1 Mus musculus cDNA, 3 end/clone = IMAGE-21598614/clone_end = 3/gb = AW018420/ | M | 140.9 | A | 219.9 | P | 112.2 | A |
| | | gi = 5871950/ug = Mm 20169/len/STRA = for | | | | | | | |
| 4 | 92913_at | Cluster Incl Z48670: M. musculus mRNA for ALDR/cds = (203,2428)/gb = Z48670/gi = 1107731/ug = Mm.4817/len = 2531/ | A | 108.2 | P | 221.5 | P | 36.4 | A |
| | | STRA = for | | | | | | | |
| 4 | 93634_at | Cluster Incl AW125157: UI-M-BH2-1-apz-d-12-0-UI s1 Mus musculus cDNA, 3 end/clone = UI-M-BH2-1-apz-12-0-UI/ | P | 396.2 | M | 516.9 | A | 445.7 | M |
| | | clone_end = 3/gb = AW125157/gi = 6100687/ug = Mm 76794/len = 484/STRA = for | | | | | | | |
| 4 | 93889_f_at | Cluster Incl X90778: M. musculus H2B gene/cds = (0,383)/gb = X90778/gi = 1518337/ug = Mm 89080/len = 384/STRA = rev | P | 64.3 | P | 84.3 | P | 59.5 | A |
| 4 | 95904_at | Cluster Incl AI835760 UI-M-Al0-aan-h-04-0-UI.s1 Mus musculus cDNA, 3 end/clone = UI-M-Al0-aan-h-04-0-UI/ | P | 303.4 | M | 510.9 | P | 393.8 | A |
| | | clone_end = 3/gb = AI835760/gi = 5469973/ug = Mm 24347/len = 490/STRA = for | | | | | | | |
| 4 | 97703_at | Cluster Incl AI427631 mj85e04 x1 Mus musculus cDNA, 3 end/clone = IMAGE-482910/clone_end = 3/gb = AI427631/ | P | −0.2 | P | 96.6 | P | −19 | M |
| | | gi = 4273557/ug = Mm 38034/len = 290/STRA = rev | | | | | | | |
| 5 | 92568_at | Cluster Incl M74555 House-keeping protein 1/cds = (87,1277)/gb = M74555/gi = 193870/ug = Mm 103/len = 2415/STRA = for | P | 323.4 | P | 224.1 | P | 194.8 | P |
| 5 | 92581_at | Cluster Incl U07159 Acetyl coenzyme A dehydrogenase, medium chain/cds = (7,1272)/gb = U07159/gi = 463908/ | P | 1516.7 | P | 552.6 | P | 674.5 | P |
| | | ug = Mm 10530/len = 1846/STRA = for | | | | | | | |
| 5 | 92640_at | Cluster Incl U47024: Materrial embryonic message 3/cds = (137,2401)/gb = U47024/gi = 1354049/ug = Mm 1178/len = 3128/ | P | 1267 | P | 929.3 | P | 915.8 | P |
| | | STRA = for | | | | | | | |
| 5 | 92773_at | Cluster Incl AF079528 Mus musculus IER5 (ler5) gene, complete cds/cds = (0,926)/gb = AF079528/gi = 3386543/ | P | 360.6 | P | 176.2 | P | 273.3 | P |
| | | ug = Mm 12246/len = 927/STRA = for | | | | | | | |
| 5 | 92789_f_at | Cluster Incl Y12474 M.musculous mRNA for centrin gene/cds = (0,503)/gb = Y12474/gi = 2246423/ug = Mm.12481/len = 504 | P | 443.3 | P | 279 | P | 243.8 | P |
| | | STRA = for | | | | | | | |
| 5 | 92818_at | Cluster Incl AF077330 Mus musculus NEDD8-conjugating enzyme (Uba3) mRNA, complete cds/cds = (39,1364)/ | P | 442.9 | P | 216.4 | P | 209.6 | P |
| | | gb = AF077330/gi = 3335647/ug = Mm 12892/len = 2117/STRA = for | | | | | | | |
| 5 | 93025_at | Cluster Incl AI835257 UI-M-AQ0-aab-b-03-0-UI s1 Mus musculus cDNA, 3 end/clone = UI-M-AQ0-aab-b-03-0-UI/ | P | 1266.4 | P | 793.2 | P | 982 | P |
| | | clone_end = 3/gb = AI835257/gi = 5469470/ug = Mm 1479/len = 342/STRA = for | | | | | | | |
| 5 | 93081_at | Cluster Incl U35142: Mus mussculus retinoblastoma-binding protein (mRbAp46) mRNA, complete cds/cds = (259,1536)/ | P | 2415 | P | 1910.4 | P | 2235.7 | P |
| | | gb = U35142/gi = 106276/ug = Mm.1603/len = 1748/STRA = for | | | | | | | |
| 5 | 93082_at | Cluster Incl AW124081 UI-M-BH2 1-apn-g-11-0-UI.s1 Mus musculus cDNA, 3 end/clone = UI-M-BH2 1-apn-g-11-0-UI/ | P | 2608.8 | P | 1538.2 | P | 2043.7 | P |
| | | clone_end = 3/gb = AW124081/gi = 6099611/ug = Mm.1608/len = 417/STRA = for | | | | | | | |
| 5 | 93088_at | Cluster Incl X01838 Beta-2 microglobulin/cds = (52,411)/gb = X01838/gi = 50104/ug = Mm 163/len = 647/STRA = for | P | 2360.4 | P | 710.4 | P | 624.2 | P |
| 5 | 93089_at | Cluster Incl X12507 Eukaryotic translation initiation facto 4A2/cds = (36,1259)/gb = X12507/gi = 50822/ug = Mm 16323/ | P | 1819 | P | 614.3 | P | 735.6 | P |
| | | len = 1894/STRA = for | | | | | | | |
| 5 | 93118_at | Cluster Incl AI844131 UI-M-AL1-ahj-d-09-0-UI s1 Mus musculus cDNA, 3 end/clone = UI = M-AL1-ahj-d-09-0-UI/ | P | 641.8 | P | 183.8 | P | 152.5 | P |
| | | clone_end = 3/gb = AI844131/gi = 5488037/ug Mm 16767/len = 425/STRA = for | | | | | | | |
| 5 | 93267_at | Cluster Incl AA688834: vs04a06 r1 Mus musculus cDNA, 5 end/clone = IMAGE-1137202/clone_end = 5/gb = AA688834/ | P | 1091.8 | P | 748.6 | P | 613.2 | P |
| | | gi = 2678263/ug = Mm 17307/len = 514/STRA = for | | | | | | | |
| 5 | 93274_at | Cluster Incl M38381 CDC-like kinase/cds = (85,1536)/gb = M38381/gi = 201070/ug = Mm 1761/len = 1740/STRA = for | P | 1215.1 | P | 176.3 | P | 148.7 | P |
| 5 | 93278_at | Cluster Incl M91458.Sterol carrier protein 2, liver/cds = (15,1658)/gb = M91458/gi = 293793/ug-Mm 1779/len = 2152/ | P | 1000.3 | P | 525.1 | P | 468.5 | P |
| | | STRA = for | | | | | | | |
| 5 | 93337_at | Cluster Incl U10119: Mus musculus SKD1 protein mRNA, complete cds/cds = (229,1563)/gb = U10119/gi = 557875/ | P | 1140.7 | P | 649 | P | 551.6 | P |
| | | ug = Mm 18705/len = 2844/STRA = for | | | | | | | |
| 5 | 93488_at | Cluster Incl AI840579 UI-M-AM0-adp-h-12-0-UI.s1 Mus musculus cDNA, 3 end/clone = UI-M-AM0-adp-h-12-0-UI/ | P | 319.5 | P | 230.1 | P | 277.1 | P |
| | | clone_end = 3/gb = AI840579/gi = 5474792/ug = Mm.19015/len = 272/STRA = for | | | | | | | |
| 5 | 93491_f_at | Cluster Incl AI836623 UI-M-AP0-abh-a-10-0-UI s1 Mus musculus cDNA, 3 end/clone = UI-M-AP0-abh-a-10-0-UI/ | P | 604.3 | P | 514 | P | 488 | P |
| | | clone_end = 3/gb = AI836623/gi = 5470836/ug = Mm 19060/len = 523/STRA = for | | | | | | | |
| 5 | 93493_at | Cluster Incl X65627 D-E-A-D (aspartate-glutamate-alanine-aspartate) box polypeptide 5/cds = (160,2004)/gb = X65627/ | P | 1169.8 | P | 643.4 | P | 794.9 | P |
| | | gi = 51262/ug = Mm.19101/len = 2318/STRA = for | | | | | | | |
| 5 | 93501_f_at | Cluster Incl AF058955 Mus musculus ATP-specific succinyl-CoA synthetase beta subunit (Sca) mRNA, partial cds/ | P | 916.8 | P | 460.9 | P | 454.6 | P |
| | | cds = (0,1280)/gb = AF058955/gi = 3766200/ug = Mm.19154/len = 1991/STRA = for | | | | | | | |
| 5 | 93503_at | Cluster Incl U88567: Mus musculus secreted frizzled related protein saFRP-2 (Sfrp2) mRNA, complete cds/cds = (235,1122)/ | P | 806.8 | P | 422.8 | P | 528.7 | P |
| | | gb = U88567/gi = 1946342/ug = Mm 19155/len = 2028 STRA = for | | | | | | | |
| 5 | 93546_s_at | Cluster Incl D14572 Mouse mRNA for PEBP2b1 protein, complete cds/cds = (232,795)/gb = D14572/gi = 303693/ | P | 1520.4 | P | 1204.6 | P | 1224 | P |
| | | ug = Mm 2018/len = 2805/STRA = for | | | | | | | |
| 5 | 93572_at | Cluster Incl AI835051 UI-M-AI1-afn-e-02-0-UI s1 Mus musculus cDNA, 3 end/clone-UI-M-AI1-afn-o-02-0-UI/clone_end = 3/ | P | 838.1 | P | 582.4 | P | 476.9 | P |
| | | gb = AI835051/gi = 5469264/ug = Mm.2041/len = 180/STRA = for | | | | | | | |
| 5 | 93773_f_at | Cluster Incl AI835041 UI-M-AI1-afn-d-02-0-UI s1 Mus musculus cDNA, 3 end/clone = UI-M-AI1-afn-d-02-0-UI/clone_end = 3/ | P | 845.6 | P | 436.1 | P | 320.6 | P |
| | | gb = AF835041/gi = 5469254/ug = Mm 21203/len = 409/STRA = for | | | | | | | |
| 5 | 93784_at | Cluster Incl AB010828 Mus musculus mRNA for BCNT, complete cds/cds = (103,990)/gb = AB010828/gi = 3327263/ | P | 467.6 | P | 383.5 | P | 296.4 | P |
| | | ug = Mm.21310/len = 1215/STRA = for | | | | | | | |
| 5 | 93794_at | Cluster Incl AI846393 UI-M-AN1-afa-h-03-0-UI s1 Mus musculus cDNA, 3 end/clone = UI-M-AN1-afa-h-03-0-UI/ | P | 610.1 | P | 339.3 | P | 450.5 | P |
| | | clone_end = 3/gb = AI846393/gi = 5490299/ug = Mm 21396/len = 193/STRA = for | | | | | | | |
| 5 | 93806_at | Cluster Incl AI848671 UI-M-AH1-agv-e-02-0-UI s1 Mus musculus cDNA, 5 end/clone = UI-M-AH1-agv-e-20-0-UI/ | P | 866 | P | 374.9 | P | 354.7 | P |
| | | clone_end = 3/gb = AI848671/gi = 5492577/ug = Mm.21446/len = 385/STRA = for | | | | | | | |
| 5 | 93986_at | Cluster Incl AA939576: vz51e01 r1 Mus musculus cDNA, 5 end/clone = IMAGE-1330008/clone_end = 5/gb = AA939576/ | P | 604.3 | P | 443.6 | P | 460.3 | P |
| | | gi = 3100353/ug = Mm 21714/len = 400/STRA = for | | | | | | | |
| 5 | 93990_at | Cluster Incl Y14196 Mus musculus mRNA for heterogeneous nuclear ribonucleoprotein H/cds = (82,1431)/gb = Y14196/ | P | 1912.1 | P | 875.9 | P | 490.8 | P |
| | | gb = 2253040/ug = Mm.21740/len = 2181/STRA = for | | | | | | | |
| 5 | 94081_at | Cluster Incl AW124390 UI-M-BH2 1-ape-c-03-0-UI s1 Mus musculus cDNA, 3 end/clone = UI-M-BH2 1-ape-c-03-0-UI/ | P | 469.4 | P | 180.1 | P | 91.2 | P |
| | | clone_end = 3/gb = AW124390/gi = 6099920/ug = Mm 22162/len = 217/STRA = for | | | | | | | |
| 5 | 94250_at | Cluster Incl U14172 Mus musculus p162 protein mRNA, complete cds/cds = (126,4160)/gb = U14172/gi = 1205975/ | P | 817.2 | P | 440.6 | P | 418.2 | P |
| | | ug = Mm.2238/len = 5119/STRA = for | | | | | | | |
| 5 | 94319_at | Cluster Incl L04966 Mus musculus ras-related protein (rab18) mRNA, complete cds/cds = (4,624)/gb = L04966/gi = 388324/ | P | 1326.3 | P | 927.8 | P | 886.2 | P |
| | | ug = Mm 22660/len = 692/STRA = for | | | | | | | |
| 5 | 94810_at | Cluster Incl X79233: Ewing sarcoma homolog/cds = (56,2023)/gb = X79233/gi = 488512/ug = Mm.22691/len = 2188/STRA = for | P | 1127.8 | P | 836.2 | P | 719.6 | P |
| 5 | 94830_at | Cluster Incl AI854300 UI-M-BH0-ajs-f-06-0-UI.s1 Mus musculus cDNA, 3 end/clone = UI-M-BHO-ajs-f-06-0-UI/clone_end = 3/ | P | 823.4 | P | 511.5 | P | 624.1 | P |
| | | gb = AI854300/gi = 5498206/ug = Mm.22749/len = 554/STRA = for | | | | | | | |
| 5 | 95418_at | Cluster Incl AI848851 UI-M-AJ1-ahb-d-09-0 UI s1 Mus musculus cDNA, 3 end/clone = UI-M-AJ1-ahb-d-09-0-UI/ | P | 375.3 | P | 264.4 | P | 248.6 | P |
| | | clone_end = 3/gb = AI848851/gi = 5492757/ug = Mm 24348/len = 377/STRA = for | | | | | | | |
| 5 | 95432_f_at | Cluster Incl AI844034 UI-M-AL1-ahi-d-02-0-UI s1 Mus musculus cDNA, 3 end/clone = UI-M-AL1-ahi-d-02-0-UI/ | P | 651.5 | P | 516.2 | P | 548.2 | P |
| | | clone_end = 3/gb = AI844034/gi = 5487940/ug = Mm 24463/len = 404/STRA = for | | | | | | | |
| 5 | 95456_r_at | Cluster Incl U41626 Split hand/foot deleted gene 1/cds = (46,258)/gb = U41626/gi = 1209725/ug = Mm 2469/len = 432/ | P | 2080.4 | P | 1546.9 | P | 1438.2 | P |
| | | STRA = for | | | | | | | |
| 5 | 95472_f_at | Cluster Incl AI526902 uj47g07.x1 Mus musculus cDNA, 3 end/clone = IMAGE-1923132/clone_end = 3/gb = AI526902/ | P | 1909.9 | P | 1188.9 | P | 1005.6 | P |
| | | gi = 4441037/ug = Mm 24805/len = 484/STRA = for | | | | | | | |
| 5 | 95485_at | Cluster Incl D29639 Mouse embryonal carcinoma cell mRNA for 3-hydroxyacyl CoA dehydrogenase, complete cds/ | P | 1800.5 | P | 1284.7 | P | 920.7 | P |
| | | cds = (53,964)/gb = D29639/gi = 1125025/ug = Mm 2491/len = 1679/STRA = for | | | | | | | |
| 5 | 95488_at | Cluster Incl AW045756 UI-M-BH1-akt-b-04-0-UI s1 Mus musculus cDNA, 3 end/clone = UI-M-BH1-akt-b-04-0-UI/ | P | 931.7 | P | 740.8 | P | 763.8 | P |
| | | clone_end = 3/gb = AW045756/gi = 5906285/ug = Mm.25018/len = 357/STRA = for | | | | | | | |
| 5 | 95516_at | Cluster Incl AB027290 Mus musculus SID 99 mRNA for small GTP binding protein, complete cds/cds = (215,820)/ | P | 1143.1 | P | 730.9 | P | 705.8 | P |
| | | gb = AB027290/gi = 5931815/ug = Mm 25306/len = 1276/STRA = for | | | | | | | |
| 5 | 96029_at | Cluster Incl AI020542 ub24g02.r1 Mus musculus cDNA, 5 end/clone = IMAGE-1378706/clone_end = 5/gb = AI020542/ | P | 463.9 | P | 393.4 | P | 406.5 | P |
| | | gi = 3234878/ug = Mm 25779/len = 374/STRA = for | | | | | | | |
| 5 | 96096_f_at | Cluster Incl AI648018 uk39c03.x1 Mus musculus cDNA, 3 end/clone = IMAGE-1971364/clone_end = 3/gb = AI648018/ | P | 297.6 | P | 211.7 | P | 255.4 | P |
| | | gi = 4726696/ug = Mm 26772/len = 543/STRA = for | | | | | | | |
| 5 | 96112_at | Cluster Incl AI651178 UI-M-BH0-akg-g-09-UI s1 Mus musculus cDNA, 3 end/clone = UI-M-BH0-akg-g-09-0-UI/ | P | 1442.1 | P | 951.7 | P | 1029.8 | P |
| | | clone_end = 3/gb = AI851178/gi = 5495049/ug = Mm.26949/len = 515/STRA = for | | | | | | | |
| 5 | 96809_at | Cluster Incl AW122195 UI-M-BH2.2-aov-b-06-0-UI s1 Mus musculus cDNA, 3 end/clone = UI-M-BH2 2-aov-b-06-0-UI/ | P | 873.8 | P | 420 | P | 405.2 | P |
| | | clone_end = 3/gb = AW122195/gi = 6097650/ug = Mm 27074/len = 589/STRA = for | | | | | | | |
| 5 | 96651_at | Cluster Incl AF035263 Mus musculus BAF57 (Baf57) gene, complete cds/cds = (0,1235)/gb = AF035283/gi = 2614754/ | P | 243.7 | P | 161.1 | P | 165.5 | P |
| | | ug = Mm 27330/len = 1236/STRA = for | | | | | | | |
| 5 | 96665_at | Cluster Incl AW211618 uo84d01 y1 Mus musculus cDNA, 5 end/clone = IMAGE-2649217/clone_end = 5/gb = AW211618/ | P | 533.3 | P | 355.6 | P | 390.7 | P |
| | | gi = 6517659/ug = Mm 27373/len = 442/STRA = for | | | | | | | |
| 5 | 96688_at | Cluster Incl AI845463 UI-M-AO1-aej-g-06-0-UI.s1 Mus musculus cDNA, 3 end/clone = UI-M-AO1-aej-g-06-0-UI/ | P | 188.7 | P | 85 | P | 93.6 | P |
| | | clone_end = 3/gb = AI845463/gi = 5489369/ug = Mm 27487/len = 423/STRA = for | | | | | | | |
| 5 | 97255_at | Cluster Incl AW061318 UI-M-BH1-anw-e-02-0-UI s1 Mus musculus cDNA, 3 end/clone = UI-M-BH1-anw-e-02-0-UI/ | P | 443.8 | P | 329 | P | 254 | P |
| | | clone_end = 3/gb = AW061318/gi = 6009069/ug = Mm 27860/len = 402/STRA = for | | | | | | | |
| 5 | 97279_at | Cluster Incl AI837615 UI-M-AK0-adg-d-01-0-UI s1 Mus musculus cDNA, 3 end/clone = UI-M-AK0-adg-d-01-0-UI/ | P | 450.9 | P | 299.5 | P | 328.9 | P |
| | | clone_end = 3/gb = AI837615/gi = 5471828/ug = Mm 27959/len = 467/STRA = for | | | | | | | |
| 5 | 97302_at | Cluster Incl AI854285.UI-M-BH0-ajs-e-02-0-UI s1 Mus musculus cDNA, 3 end/clone = UI-M-BH0-ajs-e-02-0-UI/ | P | 728.9 | P | 419.8 | P | 400.5 | P |
| | | clone_end = 3/gb = AI854285/gi = 5498191/ug = Mm 28049/len = 427/STRA = for | | | | | | | |
| 5 | 97304_at | Cluster Incl AI836100: UI-M-AJ0-abb-h-02-0-UI s1 Mus musculus cDNA, 3 end/clone = UI-M-AJ0-abb-h-02-0-UI/ | P | 545.4 | P | 360.1 | P | 304.6 | P |
| | | clone_end = 3/gb = AI836100/gi = 5470313/ug = Mm 28052/len = 358/STRA = for | | | | | | | |
| 5 | 97812_at | Cluster Incl AF006465 Mus musculus B cell antigen receptor Ig beta associated protein 1 (IBAP-1) mRNA, complete cds/ | P | 333.2 | P | 176.7 | P | 241.3 | P |
| | | cds = (45,2006)/gb = AF006465/gi = 4101717/ug = Mm 28165/len = 2190/STRA = for | | | | | | | |
| 5 | 97838_at | Cluster Incl AA684508 vr02b07.r1 Mus musculus cDNA, 5 end/clone = IMAGE-1110613/clone_end = 5/gb = AA684508/ | P | 1509.3 | P | 345.3 | P | 590.5 | P |
| | | gi = 2670225/ug = Mm 28239/len = 398/STRA = for | | | | | | | |
| 5 | 97839_at | Cluster Incl AI841960.UI-M-AH0-acu-c-10-0-UI.s2 Mus musculus cDNA, 3 end/clone = UI-M-AH0-acu-c-10-0-UI/ | P | 1895.6 | P | 1318.2 | P | 1407.6 | P |
| | | clone_end = 3/gb = AI841960/gi = 5476173/ug = Mm 28240/len = 335/STRA = for | | | | | | | |
| 5 | 97880_at | Cluster Incl AW061024: UI-M-BH1-amn-g-03-0-UI s1 Mus musculus cDNA, 3 end/clone = UI-M-BH1-amn-g-03-0-UI/ | P | 754.1 | P | 475.1 | P | 633.6 | P |
| | | clone_end = 3/gb = AW061024/gi = 6008775/ug = Mm 28365/len = 457/STRA = for | | | | | | | |
| 5 | 97896_i_at | Cluster Incl AW125218: UI-M-BH2.1-aps-c-06-0-UI s1 Mus musculus cDNA, 3 end/clone = UI-M-BH2 1-aps-c-06-0-UI/ | P | 271.3 | P | 133.5 | P | 160.9 | P |
| | | clone_end = 3/gb = AW125218/gi = 6100748/ug = Mm 28421/len = 319/STRA = for | | | | | | | |
| 5 | 97897_at | Cluster Incl AI848435 UI-M-AH1-ags-e-03-0-UI s1 Mus musculus cDNA, 3 end/clone = UI-M-AH1-ags-e-03-0-UI/ | P | 604.2 | P | 470.7 | P | 477.8 | P |
| | | clone_end = 3/gb = AI848435/gi = 5492341/ug = Mm 28422/len = 500/STRA = for | | | | | | | |
| 5 | 98905_at | Cluster Incl AJ223782 Mus musculus CDC10 gene, promoter, exon 1 and joined CDS/cds = (68,1378)/gb = AJ223782/ | P | 1481.5 | P | 678.8 | P | 537.8 | P |
| | | gi = 2827748/ug = Mm 2858/len = 2392/STRA = for | | | | | | | |
| 5 | 98909_at | Cluster Incl AW060843: UI-M-BH1-amx-d-06-0-UI s1 Mus musculus cDNA, 3 end/clone = UI-M-BH1-amx-d-06-0-UI/ | P | 440.4 | P | 410.9 | P | 377.2 | P |
| | | clone_end = 3/gb = AW060843/gi = 6008594/ug = Mm 28591/len = 419/STRA = for | | | | | | | |
| 5 | 98946_at | Cluster Incl AF033166 Mus musculus WSB-1 mRNA, complete cds/cds = (0,1265)/gb = AF033186/gi = 2766488/ | P | 576 | P | 74.3 | P | 184.4 | P |
| | | ug = Mm 28719/len = 1266/STRA = for | | | | | | | |
| 5 | 94460_at | Cluster Incl AA691445 vs11h05.r1 Mus musculus cDNA, 5 end/clone = IMAGE-1137945/clone_end = 5/gb = AA691445/ | P | 386.4 | P | 238.2 | P | 102.7 | P |
| | | gi = 2692383/ug = Mm 28821/len = 500/STRA = for | | | | | | | |
| 5 | 94505_at | Cluster Incl AW124934 UI-M-BH2 1-apu-g-11-0-UI.s1 Mus musculus cDNA, 3 end/clone = UI-M-BH2.1-apu-g-11-0-UI/ | P | 732.3 | P | 473.1 | P | 552 | P |
| | | clone_end = 3/gb = AW124934/gi = 6100464/ug = Mm 28957/len = 425/STRA = for | | | | | | | |
| 5 | 94507_at | Cluster Incl U15977 Mus musculus long chain fatty acyl CoA synthetase mRNA, complete cds/cds = (0,2099)/gb = U15977/ | P | 1343 | P | 656.6 | P | 568.6 | P |
| | | gi = 563830/ug = Mm 28962/len = 2100/STRA = for | | | | | | | |
| 5 | 95077_at | Cluster Incl U12922 RAB geramylgeranyl transferase, b subunit/cds = (0,1019)/gb = U12922/gi = 1345081/ug = Mm 29211/ | P | 708.4 | P | 452.5 | P | 459.3 | P |
| | | len = 1174/STRA = for | | | | | | | |
| 5 | 95096_at | Cluster Incl AI846695 UI-M-AN1-afi-f-06-0-UI s1 Mus musculus cDNA, 3 end/clone = UI-M-AN1-afi-f-06-0-UI/clone_end = 3/ | P | 1278.3 | P | 845 | P | 1096.6 | P |
| | | gb = AI846695/gi = 5490601/ug = Mm.29313/len = 374/STRA = for | | | | | | | |
| 5 | 95101_at | Cluster Incl AI834772: UI-M-AM0-ads-b-10-0-UI s1 Mus musculus cDNA, 3 end/clone = UI-M-AM0-ads-b-10-0-UI/ | P | 535.3 | P | 309.5 | P | 466.9 | P |
| | | clone_end = 3/gb = AI834772/gi = 5469005/ug = Mm 29344/len = 401/STRA = for | | | | | | | |
| 5 | 95647_f_at | Cluster Incl AI485345: vw50c09 y1 Mus musculus cDNA, 5 end/clone = IMAGE-1247248/clone_end = 5/gb = AI465845 | P | 1093.3 | P | 789.7 | P | 584.6 | P |
| | | gi = 4319875/ug = Mm 29500/len = 327/STRA = for | | | | | | | |
| 5 | 95648_at | Cluster Incl AA655507: vs25d01.r1 Mus musculus cDNA, 5 end/clone = IMAGE-1139233/clone_end = 5/gb = AA655507/ | P | 887.7 | P | 513.4 | P | 545.4 | P |
| | | gi = 2591661/ug = Mm.29500/len = 522/STRA = for | | | | | | | |
| 5 | 95675_at | Cluster Incl AW121182 UI-M-BH2 3-aok-a-10-0-UI s1 Mus musculus cDNA, 3 end/cone = UI-M-BH2 3-aok-a-10-0-UI/ | P | 453.2 | P | 237.3 | P | 277.4 | P |
| | | clone_end = 3/gb = AW121182/gi = 6096515/ug = Mm 29588/len = 516/STRA = for | | | | | | | |
| 5 | 96256_at | Cluster Incl M28723 Anti-oxidant protein 1/cds = (47,820)/gb = M28723/gi = 309435/ug = Mm 29821/len = 1382/STRA = for | P | 573.1 | P | 394.5 | P | 381.5 | P |
| 5 | 96268_at | Cluster Incl AI840979 UI-M-AK0-adk-e-11-0-UI s1 Mus musculus cDNA,3 end/clone = UI-M-AK0-adk-e-11-0-UI/ | P | 1227.8 | P | 1129.8 | P | 954.9 | P |
| | | clone_end = 3/gb = AI840979/gi = 5475192/ug = Mm.29845/len = 405/STRA = for | | | | | | | |
| 5 | 96862_at | Cluster Incl AI848584 UI-M-AM1-aga-d-04-0-UI s2 Mus musculus cDNA, 3 end/clone = UI-M-AM1-aga-d-04-0-UI/ | P | 2148.2 | P | 1482.6 | P | 1392.9 | P |
| | | clone_end = 3/gb = AI848584/gi = 5492490/ug = Mm 30053/len = 305/STRA = for | | | | | | | |
| 5 | 96871_at | Cluster Incl AI844355 UI-M-AL1-ahp-a-08-0-UI s1 Mus musculus cDNA, 3 end/clone = UI-M-AL1-ahp-a-08-0-UI/ | P | 601.2 | P | 426.8 | P | 307.4 | P |
| | | clone_end = 3/gb = AI844355/gi = 5488261/ug = Mm 30065/len = 330/STRA = for | | | | | | | |
| 5 | 96875_r_at | Cluster Incl AI841410: UI-M-AH0-acx-b-06-0-UI s1 Mus musculus cDNA, 3 end/clone = UI-M-AH0-acx-b-06-0-UI/ | P | 235.4 | P | 85.1 | P | 110.9 | P |
| | | clone_end = 3/gb = AI841410/gi = 5475623/ug = Mm 30069/len = 286/STRA = for | | | | | | | |
| 5 | 96881_at | Cluster Incl AW049394 UI-M-BH1-ane-e-07-0-UI s1 Mus musculus cDNA, 3 end/clone = UI-M-BH1-ane-e-07-0-UI/ | P | 527.6 | P | 410 | P | 439.4 | P |
| | | clone_end = 3/gb = AW049394/gi = 5909923/ug = Mm 30077/len = 543/STRA = for | | | | | | | |
| 5 | 96886_at | Cluster Incl AW060556 UI-M-BH1-ann-h-02-0-UI s1 Mus musculus cDNA, 3 end/clone = UI-M-BH1-ann-h-02-0-UI/ | P | 316.1 | P | 146.8 | P | 159.5 | P |
| | | clone_end = 3/gb = AW060556/gi = 6008307/ug = Mm.30083/len = 319/STRA = rev | | | | | | | |
| 5 | 96909_at | Cluster Incl AI849803 UI-M-AL1-aho-b-03-0-UI s1 Mus musculus cDNA, 3 end/clone = UI-M-AL1-aho-b-03-0-UI/ | P | 1092.5 | P | 878 | P | 847.5 | P |
| | | clone_end = 3/gb = AI849803/gi = 5493709/ug = Mm.3014/len = 501/STRA = for | | | | | | | |
| 5 | 97448_at | Cluster Incl AI845165 UI-M-BG0-ahs-g-10-0-UI s1 Mus musculus cDNA, 3 end/clone = UI-M-BG0-ahs-g-10-0-UI/ | P | 994.7 | P | 475.5 | P | 426.2 | P |
| | | clone_end = 3/gb = AI845165/gi = 5489071/ug = Mm.30245/len = 214/STRA = for | | | | | | | |
| 5 | 97491_at | Cluster Incl AW123032 UI-M-BH2 1-apb-g-02-0-UI s1 Mus musculus cDNA, 3 end/clone = UI-M-BH2 1-apb-g-02-0-UI/ | P | 391.3 | P | 237.9 | P | 274.2 | P |
| | | clone_end = 3/gb = AW123032/gi = 6098562/ug = Mm 31051/len = 361/STRA = for | | | | | | | |
| 5 | 97502_at | Cluster Incl U73445: Dihydrolipoamide dehydrogenase/cds = (109,1638)/gb = U73445/gi = 2078521/ug = Mm 3131/len = 2163/ | P | 2138.2 | P | 1262.1 | P | 972.3 | P |
| | | STRA = for | | | | | | | |
| 5 | 98065_at | Cluster Incl AI837679 UI-M-AK0-adj-b-10-0-UI.s1 Mus musculus cDNA, 3 end/clone = UI-M-AK0-adj-b-10-0-UI/ | P | 1011.7 | P | 723.5 | P | 806.5 | P |
| | | clone_end = 3/gb = AI837679/gi = 5471892/ug = Mm 34416/len = 159/STRA = for | | | | | | | |
| 5 | 98087_at | Cluster Incl AW048562 UI-M-BH1-alx-e-05-0-UI s1 Mus musculus cDNA, 3 end/clone = UI-M-BH1-alx-e-05-0-UI/ | P | 545.5 | P | 225.8 | P | 294.2 | P |
| | | clone_end = 3/gb = AW048562/gi = 5909091/ug = Mm 34580/len = 474/STRA = for | | | | | | | |
| 5 | 98121_at | Cluster Incl D49744 Farnesyltransferase, CAAX box, alpha/cds = (15,1148)/gb = D49744/gi = 1136744/ug = Mm.3496/ | P | 804 | P | 613.1 | P | 552.9 | P |
| | | len = 1629/STRA = for | | | | | | | |
| 5 | 98127_at | Cluster Incl U16741 Capping protein alpha 2/cds = (105,965)/gb = U16741/gi = 595918/ug = Mm.3529/len = 1690/STRA = for | P | 661.5 | P | 426.7 | P | 360.3 | P |
| 5 | 98524_f_at | Cluster Incl AI848479: UI-M-AP1-agf-a-10-0-UI.s2 Mus musculus cDNA, 3 end/clone = UI-M-AP1-agf-a-10-0-UI/ | P | 856.3 | P | 523.8 | P | 166.6 | P |
| | | clone_end = 3/gb = AI848479/gi = 5492385/ug = Mm 35823/len = 420/STRA = for | | | | | | | |
| 5 | 98531_g_at | Cluster Incl AI849615: UI-M-AH1-agr-g-09-0-UI s1 Mus musculus cDNA, 3 end/clone = UI-M-AH1-agr-g-09-0-UI/ | P | 2191.6 | P | 1225.6 | P | 1312.3 | P |
| | | clone_end = 3/gb = AI849615/gi = 5493521/ug = Mm 35844/len = 337/STRA = for | | | | | | | |
| 5 | 98540_g_at | Cluster Incl AF071312 Mus musculus COP9 complex subunit 2 (COPS2) mRNA, complete cds/cds = (12,1334)/ | P | 253.7 | P | 163.2 | P | 197.8 | P |
| | | gb = AF071312/gi = 3309165/ug = Mm.3596/len = 1929/STRA = for | | | | | | | |
| 5 | 98553_at | Cluster Incl AW124175: UI-M-BH2 1-apr-h-11-0-UI s1 Mus musculus cDNA, 3 end/clone = UI-M-BH2 1-apr-h-11-0-UI/ | P | 435.9 | P | 223.9 | P | 178.5 | P |
| | | clone_end = 3/gb = AW124175/gi = 6099705/ug = Mm 36769/len = 208/STRA = for | | | | | | | |
| 5 | 98587_at | Cluster Incl X61449: M. musculus mRNA for clone DN38/cds = (0,1034)/gb = X61449/gi = 50721/ug = Mm 3797/len = 1322/ | P | 1065.9 | P | 674.8 | P | 459.4 | P |
| | | STRA = for | | | | | | | |
| 5 | 98595_at | Cluster Incl U56773 Interleukin 1 receptor-associated kinase/cds = (15,2048)/gb = U56773/gi = 1480173/ug = Mm.38241/ | P | 417.1 | P | 295.8 | P | 325.4 | P |
| | | len = 2703/STRA = for | | | | | | | |
| 5 | 99095_at | Cluster Incl M63903: Max protein/cds = (160,642)/gb = M63903/gi = 199986/ug = Mm 3931/len = 1941/STRA = for | P | 358.2 | P | 322.6 | P | 295.7 | P |
| 5 | 99577_at | Cluster Incl M57647: Mouse mast cell growth factor (MGF) mRNA, complete cds/cds = (149,970)/gb = M57647/gi = 19951/ | P | 832.8 | P | 217.7 | P | 156.2 | P |
| | | ug = Mm 4235/len = 2062/STRA = for | | | | | | | |
| 5 | 99604_at | Cluster Incl AI848713: UI-M-AJ1-ahf-h-10-0-UI.s1 Mus musculus cDNA, 3 end/clone = UI-M-AJ1-ahf-a-10-0-UI/ | P | 1052.3 | P | 632.9 | P | 712.6 | P |
| | | clone_end = 3/gb = AI848713/gi = 5492619/ug = Mm 42925/len = 437/STRA = for | | | | | | | |
| 5 | 99621_s_at | Cluster Incl AA690583: vu53c05.r1 Mus musculus cDNA, 5 end/clone = IMAGE-1195112/clone_end = 5/gb = AA690583/ | P | 314.3 | P | 272.7 | P | 258.1 | P |
| | | gi = 2691519/ug = Mm 43213/len = 424/STRA = for | | | | | | | |
| 5 | 99628_at | Cluster Incl AF020308 Mus musculus HRS gene, complete cds/cds = (0,812)/gb = AF020308/gi = 2435502/ug = Mm 43331/ | P | 992.9 | P | 156 | P | 277.6 | P |
| | | len = 813/STRA = for | | | | | | | |
| 5 | 99649_at | Cluster Incl U85414 Glutamate cysteine ligase (gamma-glutamylcysteine synthetase), catalytic/cds = (0,1913)/gb = U85414/ | P | 229.5 | P | 127.9 | P | 106.3 | P |
| | | gi = 1815761/ug = Mm 4368/len = 1914/STRA = for | | | | | | | |
| 5 | 100089_at | Cluster Incl M74227 Mouse cyclophilin C (cyp C) mRNA, complete cds/cds = (51,689)/gb = M74227/gi = 192898/ | P | 2246.8 | P | 704.6 | P | 1032.7 | P |
| | | ug = Mm 4587/len = 1287/STRA = for | | | | | | | |
| 5 | 100534_at | Cluster Incl AI183109: ub91h12 r1 Mus musculus cDNA, 5 end/clone = IMAGE-1395911/clone_end = 5/gb = AI183109/ | P | 739.9 | P | 501.1 | P | 595.6 | P |
| | | gi = 3733747/ug = Mm 5248/len = 358/STRA = for | | | | | | | |
| 5 | 100543_s_at | Cluster Incl AA620184: vo63e07.r1 Mus musculus cDNA, 5 end/clone = IMAGE-1054596/clone_end = 5/gb = AA620184/ | P | 486 | P | 259.3 | P | 339.3 | P |
| | | gi = 2524060/ug = Mm 5400/len = 507/STRA = for | | | | | | | |
| 5 | 100584_at | Cluster Incl U72941 Mus musculus annexin IV mRNA, complete cds/cds = (90,1049)/gb = U72941/gi = 1778312/ug = Mm 619/ | P | 552.2 | P | 396.2 | P | 424.1 | P |
| | | len = 1961/STRA = for | | | | | | | |
| 5 | 100608_at | Cluster Incl X95641: M. musculus mRNA for serine palmitoyltransferase subunit A/cds = (11,1432)/gb = X95641/gi = 2564281/ | P | 1376.7 | P | 1118.4 | P | 1027.4 | P |
| | | ug = Mm 6505/len = 2576/STRA = for | | | | | | | |
| 5 | 101484_at | Cluster Incl U73039 Next to the Brca1/cds = (290,3256)/gb = U73039/gi = 1815651/ug = Mm.764/len = 4444/STRA = for | P | 563.7 | P | 301.1 | P | 390.2 | P |
| 5 | 101584_at | Cluster Incl X636039 M musculus RSP-1 mRNA for p33 protein/cds = (34,867)/gb = X63039/gi = 54014/ug = Mm.905/ | P | 576.9 | P | 407.6 | P | 557.8 | P |
| | | len = 1327/STRA = for | | | | | | | |
| 5 | 101590_at | Cluster Incl AI747194 ul13a12 x1 Mus musculus cDNA, 3 end/clone = IMAGE-2076670/clone_end = 3/gb = AI747194/ | P | 1472.9 | P | 910 | P | 961.7 | P |
| | | gi = 5125458/ug = Mm 91146/len = 585/STRA = for | | | | | | | |
| 5 | 101971_at | Cluster Incl AW061255: UI-M-BH1-amj-e-08-0-UI.s2 Mus musculus cDNA, 3 end/clone = UI-M-BH1-amj-e-08-0-UI/ | P | 344.6 | P | 237.7 | P | 294.8 | P |
| | | clone_end = 3/gb = AW061255/gi = 6009006/ug = Mm.9495/len = 495/STRA = for | | | | | | | |
| 5 | 102381_at | Cluster Incl AA619207: vo73e09.r1 Mus musculus cDNA, 5 end/clone = IMAGE-1064776/clone_end = 5/gb = AA619207/ | P | 308 | P | 125.7 | P | 132 | P |
| | | gi = 2523083/ug = Mm 12166/len = 367/STRA = for | | | | | | | |
| 5 | 102427_at | Cluster Incl AI849991 UI-M-BG0-aib-d-09-0-UI s1 Mus musculus cDNA, 3 end/clone = UI-M-BG0-aib-d-09-0-UI/ | P | 848.6 | P | 504.2 | P | 745.8 | P |
| | | clone_end = 3/gb = AI849991/gi = 5493897/ug = Mm.12831/len = 446/STRA = for | | | | | | | |
| 5 | 102781_at | Cluster Incl U37351 Mus musculus Paneth cell enhanced expression PCEE mRNA, complete cds/cds = (110,670)/ | P | 1112.5 | P | 401.1 | P | 341.1 | P |
| | | gb = U37351/gi = 1276644/ug = Mm 13725/len = 1220/STRA = for | | | | | | | |
| 5 | 102850_at | Cluster Incl AF037260 Mus musculus non-receptor protein tyrosine kinase Ack mRNA, complete cds/cds = (267,3434)/ | P | 559.6 | P | 493.3 | P | 329.9 | P |
| | | gb = AF037260 gi = 2921446/ug = Mm 1483/len = 4214/STRA = for | | | | | | | |
| 5 | 102990_at | Cluster Incl AA655199: vv13b10.r1 Mus musculus cDNA, 5 end/clone = IMAGE-1211515/clone_end = 5/gb = AA655199/ | P | 2422.8 | P | 990.6 | P | 1420.7 | P |
| | | gi = 2591353/ug = Mm.1543/len = 367/STRA = for | | | | | | | |
| 5 | 103027_at | Cluster Incl AA739413 vv53b11 r1 Mus musculus cDNA, 5 end/clone = IMAGE-1226109/clone_end = 5/gb = AA739413/ | P | 360.1 | P | 269.8 | P | 266.1 | P |
| | | gi = 2775599/ug = Mm.15974/len = 388/STRA = for | | | | | | | |
| 5 | 103097_at | Cluster Incl AI844487: UI-M-AJ1-ahe-e-12-0-UI.s1 Mus musculus cDNA, 3 end/clone = UI-M-AJ1-ahe-e-12-0-UI/ | P | 241.6 | P | 195.5 | P | 174.5 | P |
| | | clone_end = 3/gb = AI844487/gi = 5488393/ug = Mm 17523/len = 363/STRA = for | | | | | | | |
| 5 | 103404_at | Cluster Incl AW122831: UI-M-BH2 1-aoz-d-08-0-UI s1 Mus musculus cDNA, 3 end/clone = UI-M-BH2 1-aoz-d-08-0-UI/ | P | 468.8 | P | 420.8 | P | 399.1 | P |
| | | clone_end = 3/gb = AW122831/gi = 6098361/ug = Mm 1878/len = 376/STRA = for | | | | | | | |
| 5 | 103574_at | Cluster Incl AI841606: UI-M-AP0-abn-h-07-0-UI s1 Mus musculus cDNA, 3 end/clone = UI-M-AP0-abn-h-07-0-UI/ | P | 764.8 | P | 653.1 | P | 610.5 | P |
| | | clone_end = 3/gb = AI841606/gi = 5475819/ug = Mm 1947/len = 378/STRA = for | | | | | | | |
| 5 | 103667_at | Cluster Incl AA866655 ud06h05 r1 Mus musculus cDNA, 5 end/clone = IMAGE-1434393/clone_end = 5/gb = AA866655/ | P | 583.9 | P | 217.6 | P | 306.8 | P |
| | | gi = 2962100/ug = Mm 20722/len = 542/STRA = for | | | | | | | |
| 5 | 103875_at | Cluster Incl AA967717: uh03f10.r1 Mus musculus cDNA, 5 end/clone = IMAGE-1616875/clone_end = 5/gb = AA967717/ | P | 225.7 | P | 119.7 | P | 141.1 | P |
| | | gi = 3141610/ug = Mm 21175/len = 433/STRA = for | | | | | | | |
| 5 | 103891_i_at | Cluster Incl AI197161: ue51h10.r1 Mus musculus cDNA, 5 end/clone = IMAGE-1494691/clone_end = 5/gb = AI197161/ | P | 1326.2 | P | 918.8 | P | 701.4 | P |
| | | gi = 3749767/ug = Mm 21288/len = 475/STRA = for | | | | | | | |
| 5 | 104056_at | Cluster Incl AI573367: ud42d02.y1 Mus musculus cDNA, 5 end/clone = IMAGE-1448547/clone_end = 5/gb = AI573367/ | P | 509 | P | 180.5 | P | 153.2 | P |
| | | gi = 4536741/ug = Mm 21532/len = 448/STRA = for | | | | | | | |
| 5 | 104063_at | Cluster Incl AI840130 UI-M-AO0-acc-a-09-0-UI s1 Mus musculus cDNA, 3 end/clone = UI-M-AO0-acc-a-09-0-UI/ | P | 217.9 | P | 172.9 | P | 162.7 | P |
| | | clone_end = 3/gb = AI840130/gi = 5474343/ug = Mm 21586/len = 421/STRA = for | | | | | | | |
| 5 | 104071_at | Cluster Incl AI852433 UI-M-BH0-ajo-g-04-0-UI.s1 Mus musculus cDNA, 3 end/clone = UI-M-BH0-ajo-g-04-0-UI/ | P | 1072.8 | P | 948.1 | P | 825.5 | P |
| | | clone_end = 3/gb = AI852433/gi = 5496304/ug = Mm 21639/len = 544/STRA = for | | | | | | | |
| 5 | 104114_at | Cluster Incl AW122450 UI-M-BH2 2-aou-a-06-0-UI.s1 Mus musculus cDNA, 3 end/clone = UI-M-BH2.2-aou-a-06-0-UI/ | P | 345.9 | P | 169.8 | P | 253.4 | P |
| | | clone_end = 3/gb = AW122450/gi = 6097969/ug = Mm 21954/len = 288/STRA = for | | | | | | | |
| 5 | 104327_at | Cluster Incl AA755234 vr92d02 r1 Mus musculus cDNA, 5 end/clone = IMAGE-136163/clone_end = 5/gb = AA755234/ | P | 430.9 | P | 275.4 | P | 283 | P |
| | | gi = 2802432/ug = Mm 22459/len = 176/STRA = for | | | | | | | |
| 5 | 104332_at | Cluster Incl AI842242: UI-M-AI1-afp-d-07-0-UI s1 Mus musculus cDNA, 3 end/clone = UI-M-AI1-afp-d-07-0-UI/clone_end = 3/ | P | 627.7 | P | 506.3 | P | 505.3 | P |
| | | gb = AI842242/gi = 5476455/ug = Mm 22484/len = 349/STRA = for | | | | | | | |
| 5 | 104367_at | Cluster Incl AW124983 UI-M-BH2 1-apv-d-05-0-UI.s1 Mus musculus cDNA, 3 end/clone = UI-M-BH2 1-apv-d-05-0-UI/ | P | 547.5 | P | 350.7 | P | 298.8 | P |
| | | clone_end = 3/gb = AW124983/gi = 6100513/ug = Mm 22627/len = 398/STRA = for | | | | | | | |
| 5 | 104602_at | Cluster Incl AW121972 UI-M-BH2 3-aoj-a-09-0-UI s1 Mus musculus cDNA, 3 end/clone = UI-M-BH2 3-aoj-a-09-0-UI/ | P | 242.7 | P | 122.5 | P | 125.2 | P |
| | | clone_end = 3/gb = A121972/gi = 6097329/ug = Mm.24110/len = 474/STRA = for | | | | | | | |
| 5 | 104609_at | Cluster Incl AA644817: vs82f12 r1 Mus musculus cDNA, 5 end/clone = IMAGE-1152815/clone_end = 5/gb = AA644817/ | P | 530.2 | P | 389.7 | P | 390.9 | P |
| | | gi = 2571246/ug = Mm 24148/len = 382/STRA = for | | | | | | | |
| 5 | 104618_at | Cluster Incl AI845819 UI-M-AO1-aeg-b-07-0-UI.s1 Mus musculus cDNA, 3 end/clone = UI-M-AO1-aeg-b-07-0-UI/ | P | 314.4 | P | 164.7 | P | 113.2 | P |
| | | clone_end = 3/gb = AI845819/gi-5489725/ug = Mm 24216/len = 353/STRA = for | | | | | | | |
| 5 | 104621_at | Cluster Incl AW046060 UI-M-BH1-akv-d-12-0-UI s1 Mus musculus cDNA, 3 end/clone = UI-M-BH1-akv-d-12-0-UI/ | P | 847.8 | P | 654.4 | P | 855.3 | P |
| | | clone_end = 3/gb = AW046060/gi = 5906589/ug = Mm 24236/len = 322/STRA = for | | | | | | | |
| 5 | 104717_at | Cluster Incl AI848330 UI-M-AH1-ago-b-10-0-UI s1 Mus musculus cDNA, 3 end/clone = UI-M-AH1-ago-b-10-0-UI/ | P | 507.1 | P | 318.2 | P | 277 | P |
| | | clone_end = 3/gb = AI848330/gi = 5492236/ug = Mm 24505/len = 456/STRA = for | | | | | | | |
| 5 | 94399_at | Cluster Incl AI843172 UI-M-AK1-aet-b-11-0-UI s1 Mus musculus cDNA, 3 end/clone = UI-M-AK1-aet-b-11-0-UI/ | P | 359.8 | P | 248.1 | P | 245.2 | P |
| | | clone_end = 3/gb = AI843172/gi = 5477385/ug = Mm 25777/len = 383/STRA = for | | | | | | | |
| 5 | 94412_at | Cluster Incl AJ223733 Mus musculus CDK2 gene intron V containing the alternatively spliced exon Ve/cds = (0,123)/ | P | 592.2 | P | 524.3 | P | 440.2 | P |
| | | gb = AJ223733/gi = 2842420/ug = Mm 260/len = 124/STRA = for | | | | | | | |
| 5 | 94413_at | Cluster Incl AW21487: UI-M-BH2 2-aon-h-09-0-UI.s1 Mus musculus cDNA, 3 end/clone = UI-M-BH2.2-aon-h-09-0-UI/ | P | 355.1 | P | 288.6 | P | 245.6 | P |
| | | clone_end = 3/gb = AW121487/gi = 6096820/ug = Mm 26089/len = 168/STRA = rev | | | | | | | |
| 5 | 94435_at | Cluster Incl AI839117: UI-M-AO0-ach-b-04-0-UI.s1 Mus musculus cDNA, 3 end/clone = UI-M-AO0-ach-b-04-0-UI/ | P | 260.9 | P | 112.6 | P | 139.2 | P |
| | | clone_end = 3/gb = AI839117/gi = 5473330/ug = Mm 26537/len = 441/STRA = for | | | | | | | |
| 5 | 94439_at | Cluster Incl AW124363 UI-M-BH2 1-apq-h-10-0-UI s1 Mus musculus cDNA, 3 end/clone = UI-M-BH2 1-apq-h-10-0-UI/ | P | 373.3 | P | 197.2 | P | 316.2 | P |
| | | clone_end = 3/gb = AW124363/gi = 6099893/ug = Mm 26564/len = 253/STRA = for | | | | | | | |
| 5 | 94975_at | Cluster Incl AI845607: UI-M-AQ1-adx-e-10-0-UI s1 Mus musculus cDNA, 3 end/clone = UI-M-AQ1-adx-e-10-0-UI/ | P | 424.2 | P | 137.7 | P | 138 | P |
| | | clone_end = 3/gb = AI845607/gi = 5489513/ug = Mm 27243/len = 370/STRA = for | | | | | | | |
| 5 | 95021_at | Cluster Incl AW124028: UI-M-BH2.1-apn-c-06-0-UI s1 Mus musculus cDNA, 3 end/clone = UI-M-BH2 1-apn-c-06-0-UI/ | P | 861 | P | 649.8 | P | 405 | P |
| | | clone_end = 3/gb = AW124028/gi = 6099558/ug = Mm 27476/len = 440/STRA = for | | | | | | | |
| 5 | 96130_at | Cluster Incl AI822531: vw13g10 r1 Mus musculus cDNA, 5 end/clone = IMAGE-1243746/clone_end = 5/gb = AA822531/ | P | 1135.7 | P | 479.6 | P | 589.2 | P |
| | | gi = 2892399/ug = Mm 28133/len = 426/STRA = for | | | | | | | |
| 5 | 96191_at | Cluster Incl AI852124: UI-M-BH0-ajb-c-05-0-UI s1 Mus musculus cDNA, 3 end/clone = UI-M-BH0-ajb-c-05-0-UI/ | P | 717.9 | P | 492.2 | P | 491.7 | P |
| | | clone_end = 3/gb = AI852124/gi = 546030/ug = Mm 28512/len = 275/STRA = for | | | | | | | |
| 5 | 96192_at | Cluster Incl AF062567: Mus musculus transcription factor Sp3 mRNA, partial cds/cds = (0,2177)/gb = AF062567/gi = 3132609/ | P | 857.5 | P | 493.6 | P | 533 | P |
| | | ug = Mm 28513/len = 3616/STRA = for | | | | | | | |
| 5 | 96199_at | Cluster Incl AF073996 Mus musculus myotubularin (Mtm1) mRNA, complete cds/cds = (75,1886)/gb = AF073996/ | P | 166 | P | 110.7 | P | 130.6 | P |
| | | gi = 3882999/ug = Mm.28580/len = 3339/STRA = for | | | | | | | |
| 5 | 96238_at | Cluster Incl AI853996: UI-M-BH0-aiv-g-11-0-UI.s1 Mus musculus cDNA, 3 end/clone = UI-M-BH0-aiv-g-11-0-UI/ | P | 315.1 | P | 170.1 | P | 131.9 | P |
| | | clone_end = 3/gb = AI853996/gi = 5497902/ug = Mm.28807/len = 255/STRA = for | | | | | | | |
| 5 | 96726_at | Cluster Incl AW049619: UI-M-BH1-and-d-10-0-UI.s1 Mus musculus cDNA, 3 end/clone = UI-M-BH1-and-d-10-0-UI/ | P | 428.1 | P | 291.6 | P | 202.7 | P |
| | | clone_end = 3/gb = AW049619/gi = 5910148/ug = Mm.28837/len = 423/STRA = for | | | | | | | |
| 5 | 96751_at | Cluster Incl AI842674 UI-M-AO1-aeo-a-12-0-UI.s1 Mus musculus cDNA, 3 end/clone = UI-M-AO1-aeo-a-12-0-UI/ | P | 1584.3 | P | 1087.1 | P | 1177.9 | P |
| | | clone_end = 3/gb = AI842674/gi = 5476887/ug = Mm.28972/len = 341/STRA = for | | | | | | | |
| 5 | 96762_at | Cluster Incl AW046160 UI-M-BH1-akw-b-02-0-UI s1 Mus musculus cDNA, 3 end/clone = UI-M-BH1-akw-b-02-0-UI/ | P | 1029.5 | P | 691.8 | P | 606 | P |
| | | clone_end = 3/gb = AW046160/gi = 5906689/ug = Mm 29005/len = 532/STRA = rev | | | | | | | |
| 5 | 96777_at | Cluster Incl AW049372: UI-M-BH1-ane-b-10-0-UI.s1 Mus musculus cDNA, 3 end/clone = UI-M-BH1-ane-b-10-0-UI/ | P | 472.4 | P | 295.7 | P | 283.7 | P |
| | | clone_end = 3/gb = AW049372/gi = 5909901/ug = Mm 29056/len = 322/STRA = for | | | | | | | |
| 5 | 97948_at | Cluster Incl M26391 Retinoblastoma 1/cds = (98,2863)/gb = M26391/gi = 200452/ug = Mm.304/len = 4591/STRA = for | P | 176.6 | P | 105.8 | P | 162.7 | P |
| 5 | 98455_at | Cluster Incl U46026 Activating transcription factor 2/cds = (0,731)/gb = U46026/gi = 1655802/ug = Mm 3466/len = 969/ | P | 363.4 | P | 118 | P | 286.3 | P |
| | | STRA = for | | | | | | | |
| 5 | 98503_at | Cluster Incl U53586 Ecotropic viral integration site 5/cds = (68,2497)/gb = U53586/gi = 1777918/ug = Mm 35796/len = 5870/ | P | 380.9 | P | 248.4 | P | 297 | P |
| | | STRA = for | | | | | | | |
| 5 | 98981_s_at | Cluster Incl X64840: Transcription factor 12/cds = (66,2186)/gb = X64840/gi = 49924/ug = Mm 36894/len = 3007/STRA = for | P | 847.4 | P | 668.6 | P | 714 | P |
| 5 | 99013_f_at | Cluster Incl AI846797: UI-M-AO1-ael-h-03-0-UI.s1 Mus musculus cDNA, 3 end/clone = UI-M-AO1-ael-h-03-0-UI/ | P | 2763.7 | P | 2043.5 | P | 2070.6 | P |
| | | clone_end = 3/gb = AI846797/gi = 5490703/ug = Mm 38445/len = 383/STRA = for | | | | | | | |
| 5 | 99070_at | Cluster Incl U12473 Conserved helix-loop-helix ubiquitous kinase/cds = (53,2290)/gb = U12473/gi = 1079492/ug = Mm 3996/ | P | 442.7 | P | 237 | P | 188.4 | P |
| | | len = 3466/STRA = for | | | | | | | |
| 5 | 99491_at | Cluster Incl U53696 interleukin 10 receptor, beta/cds = (2,1051)/gb = U53696/gi = 1305488/ug = Mm.4154/len = 1670/ | P | 913.1 | P | 445.5 | P | 629 | P |
| | | STRA = for | | | | | | | |
| 5 | 99493_at | Cluster Incl AI842066 UI-M-AN1-afg-a-11-0-UI s1 Mus musculus cDNA, 3 end/clone = UI-M-AN1-afg-a-11-0-UI/ | P | 195.5 | P | 135.2 | P | 149.2 | P |
| | | clone_end = 3/gb = AI842066/gi = 5476279/ug = Mm 41555/len = 302/STRA = for | | | | | | | |
| 5 | 99513_at | Cluster Incl M72394 Mouse calcium-dependent phospholipid binding protein (PLA2) mRNA, complete cds/cds = (108,2354)/ | P | 299.9 | P | 137 | P | 175.6 | P |
| | | gb = M72394/gi = 200390/ug = Mm.4186/len = 2787/STRA = for | | | | | | | |
| 5 | 99552_at | Cluster Incl U79550.Slug, chicken homolog/cds = (43,852)/gb = U79550/gi = 1711202/ug = Mm 4272/len = 1945/STRA = for | P | 1157.6 | P | 578.9 | P | 657.7 | P |
| 5 | 99955_at | Cluster Incl U70674: Mus musculus m-Numb (m-nb) mRNA, complete cds/cds = (166,1947)/gb = U70674/gi = 1575755/ | P | 229.1 | P | 121.9 | P | 153.9 | P |
| | | ug = Mm 4390/len = 3331/STRA = for | | | | | | | |
| 5 | 99964_at | Cluster Incl AW061016 UI-M-BH1-amn-f-06-0-UI s1 Mus musculus cDNA, 3 end/clone = UI-M-BH1-amn-f-06-0-UI/ | P | 161.6 | P | 109.4 | P | 76.2 | P |
| | | clone_end = 3/gb = AW061016/gi = 6008767/ug = Mm 44170/len = 435/STRA = for | | | | | | | |
| 5 | 99994_at | Cluster Incl AF041376: Mus musculus cell death activator CIDE-A (Cide-a) mRNA, complete cds/cds = (213,868)/ | P | 1059 | P | 582 | P | 416.9 | P |
| | | gb = AF041376/gi = 3114591/ug = Mm 449/len = 1114/STRA = for | | | | | | | |
| 5 | 100019_at | Cluster Incl D45889 Chondroitin sulfate proteoglycan 2/cds = (0,124)/gb = D45889/gi = 994777/ug = Mm.4575/len = 2201/ | P | 804.1 | P | 355 | P | 344.3 | P |
| | | STRA = for | | | | | | | |
| 5 | 100878_at | Cluster Incl AW124985: UI-M-BH2.1-apv-d-07-0-UI.s1 Mus musculus cDNA, 3 end/clone = UI-M-BH2-1-apv-d-07-0-UI/ | P | 598.8 | P | 289.1 | P | 377.4 | P |
| | | clone_end = 3/gb = AW124985/gi = 6100515/ug = Mm 531/len = 339/STRA = for | | | | | | | |
| 5 | 100905_at | Cluster Incl AI849361 UI-M-AJ1-aha-g-09-0-UI s1 Mus musculus cDNA, 3 end/clone = UI-M-AJ1-aha-g-09-0-UI/ | P | 360 | P | 215.7 | P | 166.4 | P |
| | | clone_end = 3/gb = AI849361/gi = 5493267/ug = Mm 5772/len = 132/STRA = for | | | | | | | |
| 5 | 100951_at | Cluster Incl AF014010.Polycystic kidney disease 2/cds = (83,2983)/gb = AF014010/gi = 2558834/ug = Mm 6442/len = 5134/ | P | 353.5 | P | 116.8 | P | 245.8 | P |
| | | STRA = rev | | | | | | | |
| 5 | 100957_at | Cluster Incl AA881160 vz07b02 r1 Mus musculus cDNA, 5 end/clone = IMAGE-1314987/clone_end = 5/gb = AA881160/ | P | 359 | P | 268.5 | P | 211.4 | P |
| | | gi = 2990470/ug = Mm 6509/len = 575/STRA = for | | | | | | | |
| 5 | 100984_at | Cluster Incl M63725 Mouse binding protein for T-cell receptor (TCR-ATF1) mRNA, complete cds/cds = (20,829)/ | P | 474 | P | 348.7 | P | 186.9 | P |
| | | gb = M63725/gi = 201821/ug = Mm.676/len = 2122/STRA = for | | | | | | | |
| 5 | 101906_at | Cluster Incl AA032310: MTA.G11.085 A Mus musculus cDNA, 5 end/clone = MTA.G11.085/clone_end = 5/gb = AA032310/ | P | 1010.3 | P | 609.1 | P | 383.1 | P |
| | | gi = 1504156/ug = Mm.89817/len = 380/STRA = for | | | | | | | |
| 5 | 101914_at | Cluster Incl AI846484 UI-M-AN1-aff-a-11-0-UI.s1 Mus musculus cDNA, 3 end/clone = UI-M-AN1-aff-a-11-0-UI/ | P | 344.7 | P | 264.2 | P | 217.8 | P |
| | | clone_end = 3/gb = AI846484/gi = 5490390/ug = Mm.90698/len = 393/STRA = for | | | | | | | |
| 5 | 102302_at | Cluster Incl L16992 Mus musculus branched-chain alpha-ketoacid dehydrogenase E1 beta-subunit mRNA sequence/ | P | 521.9 | P | 167.4 | P | 258.5 | P |
| | | cds = UNKNOWN/gb = L16992/gi = 293303/ug = Mm 12819/len = 1390/STRA = for | | | | | | | |
| 5 | 102309_at | Cluster Incl AB012725.Mus musculus ZAN75 mRNA for zinc finger protein, complete cds/cds = (81,1823)/gb = AB012725/ | P | 212.7 | P | 98.6 | P | 135.8 | P |
| | | gi = 3298471/ug = Mm 12891/len = 2627/STRA = for | | | | | | | |
| 5 | 102644_at | Cluster Incl U13371-Mus musculus (C57BL/10 X C3H)F2 clone 1 5 novel mRNA from renin-expressing kidney tumor cell | P | 327.7 | P | 245 | P | 249.1 | P |
| | | line, partial sequence/cds = UNKNOWN/gb = U13371/gi = 987223/ug = Mm 1314/len = 1217/STRA = for | | | | | | | |
| 5 | 102710_at | Cluster Incl AF020313: Mus musculus proline-rich protein 48 mRNA, partial cds/cds = (0,895)/gb = AF020313/gi = 2624971/ | P | 295 | P | 92.9 | P | 203.4 | P |
| | | ug = Mm 14255/len = 2223/STRA = for | | | | | | | |
| 5 | 103201_at | Cluster Incl M86377: Mouse esk kinase mRNA, complete cds/cds = (37,2607)/gb = M86377/gi = 193109/ug = Mm.1904/ | P | 254.7 | P | 170.6 | P | 92.6 | P |
| | | len = 2924/STRA = for | | | | | | | |
| 5 | 103312_f_at | Cluster Incl AW047929 UI-M-BH1-aln-d-03-0-UI.s1 Mus musculus cDNA, 3 end/clone = UI-M-BH1-aln-d-03-0-UI/ | P | 577.9 | P | 429.4 | P | 344.1 | P |
| | | clone_end = 3/gb = AW047929/gi = 5908458/ug = Mm.21228/len = 461/STRA = rev | | | | | | | |
| 5 | 103440_at | Cluster Incl AW226540: up08h09.x1 Mus musculus cDNA, 3 end/clone = IMAGE-2651489/clone_end = 3/gb = AW226540/ | P | 412.5 | P | 245.2 | P | 240 | P |
| | | gi = 6555836/ug = Mm 21424/len = 462/STRA = for | | | | | | | |
| 5 | 103695_f_at | Cluster Incl AW047329 UI-M-BH1-amh-h-04-0-UI s1 Mus musculus cDNA, 3 end/clone = UI-M-BH1-amh-h-04-0-UI/ | P | 758.4 | P | 531.2 | P | 428.9 | P |
| | | clone_end = 3/gb = AW047329/gi = 5907858/ug = Mm 23994/len = 438/STRA = for | | | | | | | |
| 5 | 103704_at | Cluster Incl AW047625: UI-M-BH1-akm-f-09-0-UI s1 Mus musculus cDNA, 3 end/clone = UI-M-BH1-akm-f-09-0-UI/ | P | 299.7 | P | 194.9 | P | 207 | P |
| | | clone_end = 3/gb = AW047625/gi = 5908154/ug = Mm.24075/len = 326/STRA = for | | | | | | | |
| 5 | 103739_at | Cluster Incl AW230977 uo70h08 y1 Mus musculus cDNA, 5 end/clone = IMAGE-2647935/clone_end = 5/gb = AW230977/ | P | 365.7 | P | 244.7 | P | 346.5 | P |
| | | gi = 6560343/ug = Mm.24411/len = 424/STRA = for | | | | | | | |
| 5 | 103753_at | Cluster Incl AI159572: vz77c07.r1 Mus musculus cDNA, 5 end/clone = IMAGE-1332492/clone_end = 5/gb = AI159572/ | P | 840.1 | P | 206.3 | P | 195.6 | P |
| | | gi = 3692754/ug = Mm.24527/len = 476/STRA = for | | | | | | | |
| 5 | 103932_at | Cluster Incl AA655311: vq85f02.s1 Mus musculus cDNA, 5 end/clone = IMAGE-1109115/clone_end = 5/gb = AA655311/ | P | 648.2 | P | 512.3 | P | 414 | P |
| | | gi = 2591465/ug = Mm 25148/len = 416/STRA = for | | | | | | | |
| 5 | 103947_at | Cluster Incl AW048038 UI-M-BH1-afv-c-02-0-UI s1 Mus musculus cDNA, 3 end/clone = UI-M-BH1-alv-c-02-0-UI/ | P | 323.3 | P | 266.9 | P | 313.9 | P |
| | | clone_end = 3/gb = AW048038/gi = 5908532/ug = Mm 25345/len = 269/STRA = for | | | | | | | |
| 5 | 103969_at | Cluster Incl AA981154 vx57h02 r1 Mus musculus cDNA, 5 end/clone = IMAGE-1279347/clone_end = 5/gb = AA981154/ | P | 567.9 | P | 394.6 | P | 497.7 | P |
| | | gi = 3159690/ug = Mm 25715/len = 218/STRA = for | | | | | | | |
| 5 | 104004_at | Cluster Incl AI931876: uI67b12.x1 Mus musculus cDNA, 3 end/clone = IMAGE-2135615/clone_end = 3/gb = AI931876/ | P | 498.4 | P | 264.3 | P | 295.8 | P |
| | | gi = 5668361/ug = Mm 26610/len = 529/STRA = for | | | | | | | |
| 5 | 104015_at | Cluster Incl AW047992: UI-M-BH1-alv-e-08-0-UI s1 Mus musculus cDNA, 3 end/clone = UI-M-BHI-alv-e-08-0-UI/ | P | 558.7 | P | 477.1 | P | 405.5 | P |
| | | clone_end = 3/gb = AW047992/gi = 5908561/ug = Mm 26833/len = 478/STRA = for | | | | | | | |
| 5 | 104412_at | Cluster Incl AI153412.uc53a01 r1 Mus musculus cDNA, 5 end/clone = IMAGE-1429320/clone_end = 5/gb = AI153412/ | P | 419.9 | P | 299.6 | P | 252.3 | P |
| | | gi = 3681881/ug = Mm 28859/len = 507/STRA = for | | | | | | | |
| 5 | 104640_f_at | Cluster Incl AI464596: mz66c12 y1 Mus musculus cDNA, 5 end/clone = IMAGE-718390/clone_end = 5/gb = AI464598/ | P | 466.1 | P | 219.7 | P | 244.5 | P |
| | | gi = 4318626/ug = Mm.31114/len = 339/STRA = for | | | | | | | |
| 5 | 92302_at | Cluster Incl Z11664 Son of sevenless homolog 2, (Drosophila)/cds = (0,3893)/gb = Z11664/gi = 54136/ug = Mm 3770/ | P | 194.4 | P | 136.5 | P | 114 | P |
| | | len = 5253/STRA = for | | | | | | | |
| 5 | 92502_at | Cluster Incl X95504 M musculus mRNA for zinc finger protein/cds = UNKNOWN/gb = X95504/gi = 2828233/ug = Mm 42200/ | P | 559.2 | P | 183.6 | P | 438 | P |
| | | len = 3732/STRA = for | | | | | | | |
| 5 | 92513_at | Cluster Incl AJ002636 Mus musculus mRNA for nuclear protein SA2/cds = (279,3767)/gb = AJ002638/gi = 2644956/ | P | 530.9 | P | 143.5 | P | 177.1 | P |
| | | ug = Mm 42238/len = 3871/STRA = for | | | | | | | |
| 5 | 92660_f_at | Cluster Incl X92665 M. musculus mRNA for ubiquitin-conjugating enzyme UbcM3/cds = (153,734)/gb = X92665/gi = 107238/ | P | 761.4 | P | 558 | P | 418.4 | P |
| | | ug = Mm.4429/len = 1328/STRA = for | | | | | | | |
| 5 | 93190_at | Cluster Incl AW209179 uo02h05.x1 Mus musculus cDNA, 3 end/clone = IMAGE-2582265/clone_end = 3/gb = AW209179/ | P | 346.6 | P | 188.7 | P | 218 | P |
| | | gi = 6515119/ug = Mm 5547/len = 428/STRA = for | | | | | | | |
| 5 | 93877_at | Cluster Incl AI834777 UI-M-AM0-ads-c-04-0-UI s1 Mus musculus cDNA, 3 end/clone = UI-M-AM0-ads-c-04-0-UI/ | P | 504.8 | P | 326.6 | P | 291 | P |
| | | clone_end = 3/gb = AI834777/gi = 5469010/ug = Mm 886/len = 279/STRA = for | | | | | | | |
| 5 | 94732_at | Cluster Incl X99251: Repetin/cds = (65,3457)/gb = X99251/gi = 1806131/ug = Mm 1417/len = 4178/STRA = for | P | 1872.9 | A | 114.2 | P | 197.9 | P |
| 5 | 94768_at | Cluster Incl D37790 Mouse mRNA for beta-1,4-galactosyltransferase/cds = UNKNOWN/gb = D37790/gi = 517126/ | P | 1626 | P | 1304.6 | P | 1292.1 | P |
| | | ug = Mm 164/len = 3298/STRA = rev | | | | | | | |
| 5 | 95395_at | Cluster Incl AI225869 uI22e07 y1 Mus musculus cDNA, 5 end/clone = IMAGE-1852068/clone_end = 5/gb = AI225869/ | P | 284.9 | P | 222.8 | P | 208.8 | P |
| | | gi = 3808922/ug = Mm 23834/len = 388/STRA = for | | | | | | | |
| 5 | 97689_at | Cluster Incl M26071: Coagulation factor III/cds = UNKNOWN/gb = M26071/gi = 201924/ug = Mm 3742/len = 1821/STRA = for | P | 581.9 | P | 309.1 | P | 495.9 | P |
| 5 | 95787_s_at | Cluster Incl X87685 M musculus sterol camer protein 2-pseudogene/cds = (0,478)/gb = X87685/gi = 854333/ug = Mm 87814/ | P | 636.6 | P | 434.4 | P | 411.4 | P |
| | | len = 479/STRA = for | | | | | | | |
| 5 | 92542_at | Cluster Incl AI849035 UI-M-AH1-agw-a-06-0-UI s1 Mus musculus cDNA, 3 end/clone = UI-M-AH1-agw-a-06-0-UI/ | P | 1605.9 | P | 304.8 | P | 285.3 | P |
| | | clone_end = 3/gb = AI849035/gi = 5492941/ug = Mm.10009/len = 377/STRA = for | | | | | | | |
| 5 | 92579_at | Cluster Incl L00993 Sjogren syndrome antigen B/cds = (140,1387)/gb = L00993/gi = 198712/ug = Mm.10508/len = 1961/ | P | 184.6 | P | 78.2 | P | 94.3 | P |
| | | STRA = for | | | | | | | |
| 5 | 92648_at | Cluster Incl U19521: Mus musculus vesicle transport protein (munc.18c) mRNA, complete cds/cds = (159,1937)/gb = U19521/ | P | 370.3 | P | 132.3 | P | 193.1 | P |
| | | gi = 642027/ug = Mm 12155/len = 2037/STRA = for | | | | | | | |
| 5 | 93253_at | Cluster Incl D87271: Mus musculus DNA for ERK2/cds = (223,1098)/gb = D87271/gi = 2506074/ug = Mm 1700/len = 1182/ | P | 193.5 | P | 207.8 | P | 159.9 | P |
| | | STRA = for | | | | | | | |
| 5 | 95438_at | Cluster Incl AI846783 UI-M-AO1-ael-f-12-0-UI s1 Mus musculus cDNA, 3 end/clone = UI-M-AO1-ael-f-12-0-UI/ | P | 237.9 | P | 148.9 | P | 102.6 | A |
| | | clone_end = 3/gb = AI846783/gi = 5490689/ug = Mm 24541/len = 269/STRA = for | | | | | | | |
| 5 | 96616_at | Cluster Incl AI891629 ul57e11.x1 Mus musculus cDNA, 3 end/clone = IMAGE-2123180/clone_end = 3/gb = AI891629/ | P | 423.8 | P | 161.8 | P | 80.8 | A |
| | | gi = 5597531/ug = Mm 27123/len = 443/STRA = for | | | | | | | |
| 5 | 96266_at | Cluster Incl AI841982 UI-M-AHO-acu-h-04-0-UI s2 Mus musculus cDNA, 3 end/clone = UI-M-AH0-acu-h-04-0-UI/ | P | 1172 | P | 722.4 | P | 481.1 | P |
| | | clone_end = 3/gb = AI841982/gi = 5476195/ug = Mm 29841/len = 334/STRA = for | | | | | | | |
| 5 | 99194_at | Cluster Incl AW124271 UI-M-BH2 1-aph-h-11-0-UI s1 Mus musculus cDNA, 3 end/clone = UI-M-BH2.1-aph-h-11-0-UI/ | P | 142.1 | P | 88.1 | A | 114.2 | P |
| | | clone_end = 3/gb = AW124271/gi = 6099801/ug = Mm 41935/len = 363/STRA = for | | | | | | | |
| 5 | 101475_at | Cluster Incl M64068 Mouse zinc finger protein (bmi-1) gene, complete cds/cds = (0,749)/gb = M64068/gi = 192204/ | P | 460 | P | 233.8 | P | 298.8 | P |
| | | ug = Mm.7719/len = 750/STRA = for | | | | | | | |
| 5 | 101965_at | Cluster Incl AF037205 Mus musculus RING zinc finger protein (Rzf) mRNA, complete cds/cds = (88,1233)/gb = AF037205/ | P | 440.4 | P | 208.9 | P | 261.4 | P |
| | | gi = 2746334/ug = Mm.9326/len = 2240/STRA = for | | | | | | | |
| 5 | 103083_at | Cluster Incl U69543 Lipase, hormone sensitive/cds = (592,2871)/gb = U69543/gi = 1899053/ug = Mm 1721/len = 3175/ | P | 590.3 | P | 339 | P | 430.9 | P |
| | | STRA = for | | | | | | | |
| 5 | 103328_at | Cluster Incl U59864 Mus musculus TRAF-interacting protein I-TRAF mRNA, complete cds/cds = (68,1414)/gb = U59864/ | P | 316.1 | P | 115.7 | P | 68.8 | P |
| | | gi = 1518019/ug = Mm 1803/len = 1971/STRA = for | | | | | | | |
| 5 | 103596_at | Cluster Incl AF085219: Mus musculus alpha diacylglycerol kinase mRNA, complete cds/cds = (19,2211)/gb = AF085219/ | P | 172.9 | P | 64.4 | P | 88 | P |
| | | gi = 3493665/ug = Mm 18981/len = 2239/STRA = for | | | | | | | |
| 5 | 103638_at | Cluster Incl AW046460 UI-M-BH1-akp-g-06-0-UI s1 Mus musculus cDNA, 3 end/clone = UI-M-BH1-akp-g-06-0-UI/ | P | 101.1 | P | 27 | P | 59.2 | A |
| | | clone_end = 3/gb = AW046460/gi = 5906989/ug = Mm.2035/len = 278/STRA = for | | | | | | | |
| 5 | 104060_at | Cluster Incl AI845977 UI-M-AP1-agi-b-12-0-UI s1 Mus musculus cDNA, 3 end/clone = UI-M-AP1-agl-b-12-0-UI/ | P | 293.8 | P | 257 | P | 204.1 | P |
| | | clone_end = 3/gb = AI845977/gi = 5489883/ug = Mm 21568/len = 351/STRA = for | | | | | | | |
| 5 | 104070_at | Cluster Incl AW047728 UI-M-BH1-alk-a-01-0-UI s1 Mus musculus cDNA, 3 end/clone = UI-M-BH1-alk-a-01-0-UI/ | P | 380.7 | P | 147.7 | P | 127.9 | P |
| | | clone_end = 3/gb = AW047728/gi = 5908257/ug = Mm 21637/len = 392/STRA = for | | | | | | | |
| 5 | 104290_at | Cluster Incl AI847837 UI-M-AM1-afu-b-08-o-UI.s1 Mus musculus cDNA, 3 end/clone = UI-M-AM1-afu-b-08-0-UI/ | P | 266 | P | 175.3 | P | 111.4 | P |
| | | clone_end = 3/gb = AI847837/gi = 5491743/ug = Mm 22279/len = 387/STRA = rev | | | | | | | |
| 5 | 104558_at | Cluster Incl AA867123: vx23f05.r1 Mus musculus cDNA, 5 end/clone = IMAGE-1265313/clone_end = 5/gb = AA867123/ | P | 463 | P | 165.8 | P | 388.1 | P |
| | | gi = 2962568/ug = Mm.23808/len = 552/STRA = for | | | | | | | |
| 5 | 104706_at | Cluster Incl U69171: Mus musculus peroxisomal PTS2 receptor mRNA, complete cds/cds = (92,1048)/gb = U69171/ | P | 191 | P | 92.4 | P | 105.7 | A |
| | | gi = 1890656/ug = Mm 2440/len = 1468/STRA = for | | | | | | | |
| 5 | 94923_f_at | Cluster Incl AA543502 vk25c02 r1 Mus musculus cDNA, 5 end/clone = IMAGE-947618/clone_end = 5/gb = AA543502/ | P | 870.7 | P | 550.1 | P | 646.7 | P |
| | | gi = 2291979/ug = Mm 26609/len = 598/STRA = rev | | | | | | | |
| 5 | 94992_at | Cluster Incl AI840667 UI-M-AM0-adr-b-05-0-UI s1 Mus musculus cDNA, 3 end/clone = UI-M-AM0-adr-b-05-0-UI/ | P | 471.8 | P | 223.7 | P | 218.5 | P |
| | | clone_end = 3/gb = AI840667/gi = 5474875/ug = Mm 27327/len = 304/STRA = for | | | | | | | |
| 5 | 95040_at | Cluster Incl AI840810 UI-M-AH0-adb-b-09-0-UI s1 Mus musculus cDNA, 3 end/clone = UI-M-AH0-adb-b-09-0-UI/ | P | 148.4 | P | 127.9 | P | 128 | P |
| | | clone_end = 3/gb = AI840810/gi = 5475023/ug = Mm 27569/len = 347/STRA = for | | | | | | | |
| 5 | 96215_f_at | Cluster Incl AI153421 uc53a11.r1 Mus musculus cDNA, 5 end/clone = IMAGE-1429340/clone_end = 5/gb = AI153421/ | P | 1395 | P | 375.5 | P | 353.4 | P |
| | | gi = 3681890/ug = Mm 28674/len = 385/STRA = for | | | | | | | |
| 5 | 97422_at | Cluster Incl AW048882 UI-M-BH1-amf-e-12-0-UI s1 Mus musculus cDNA, 3 end/clone = UI-M-BH1-amf-e-12-0-UI/ | P | 328.5 | P | 103.5 | A | 148.3 | P |
| | | clone_end = 3/gb = AW048882/gi = 5909411/ug = Mm 29999/len = 497/STRA = for | | | | | | | |
| 5 | 97976_at | Cluster Incl L43326.Kinectin 1/cds = (19,4002)/gb = L43326/gi = 887379/ug = Mm 3110/len = 4466/STRA = rev | P | 255.3 | P | 88.2 | P | 108.7 | P |
| 5 | 103727_at | Cluster Incl AI850972 UI-M-BH0-akd-g-07-0-UI s1 Mus musculus cDNA, 3 end/clone = UI-M-BH0-akd-g-07-0-UI/ | P | 251.3 | P | 113.7 | P | 20.4 | A |
| | | clone_end = 3/gb = AI850972/gi = 5494878/ug = Mm 24291/len = 370/STRA = for | | | | | | | |
| 5 | 104193_at | Cluster Incl AW047583 UI-M-BH1-akm-b-04-0-UI a1Mus musculus cDNA, 3 end/clone = UI-M-BH1-akm-b-04-0-UI/ | P | 445.4 | P | 179.1 | P | 179.3 | P |
| | | clone_end = 3/gb = AW047583/gi = 5908112/ug = Mm 27493/len = 426/STRA = for | | | | | | | |
| 5 | 104683_at | Cluster Incl AW120783 UI-M-BH2.3-any-g-06-0-UI.s1 Mus musculus cDNA, 3 end/clone = UI-M-BH2.3-any-g-06-0-UI/ | P | 262.7 | P | 167.4 | P | 125.8 | P |
| | | clone_end = 3/gb = AW120783/gi = 6096116/ug = Mm.3289/len = 422/STRA = for | | | | | | | |
| 5 | 93203_f_at | Cluster Incl AW107463 um17a12x1 Mus musculus cDNA, 3 end/clone = IMAGE-2192542/clone_end = 3/gb = AW107463/ | P | 173.2 | P | 78.4 | A | 85.4 | P |
| | | gi = 6078263/ug = Mm.5697/len = 484/STRA = rev | | | | | | | |
| 5 | 102130_f_at | Cluster Incl C88243 C88243 Mus musculus cDNA, 3 end/clone = J0259A06/clone_end = 3/gb = C88243/gi = 2920200/ | P | 239.3 | P | 92.7 | A | 190.1 | P |
| | | ug = Mm 43522/len = 610/STRA = rev | | | | | | | |
| 5 | 96608_at | Cluster Incl AF023463 Mus musculus peroxisomal phytanoyl-CoA alpha-hydroxylase (PAHX) mRNA, complete cds/ | P | 200.9 | P | 135.7 | P | 170.4 | A |
| | | cds = (39,1055)/gb = AF023463/gi = 2564672/ug = Mm 27066/len = 1419/STRA = for | | | | | | | |
| 5 | 97853_at | Cluster Incl AW047331.UI-M-BH1-amh-h-07-0-UI s1 Mus musculus cDNA, 3 end/clone = UI-M-BH1-amh-h-07-0-UI/ | P | 198.3 | P | 110.4 | P | 81.3 | P |
| | | clone_end = 3/gb = AW047331/gi = 5907860/ug = Mm 28287/len = 333/STRA = for | | | | | | | |
| 5 | 97869_at | Cluster Incl AI844043 UI-M-AL1-ahi-d-11-0-UI s1 Mus musculus cDNA, 3 end/clone = UI-M-AL1-ahi-d-11-0-UI/ | P | 420.8 | P | 123.2 | P | 123.4 | P |
| | | clone_end = 3/gb = AI844043/gi = 5487949/ug = Mm.28336/len = 378/STRA = for | | | | | | | |
| 5 | 97892_at | Cluster Incl AI844732 UI-M-AL1-ahq-c-04-0-UI s1 Mus musculus cDNA, 3 end/clone = UI-M-AL1-ahq-c-04-0-UI/ | P | 94.5 | P | 34.2 | M | 58.1 | P |
| | | clone_end = 3/gb = AI844732/gi = 5488638/ug = Mm 28414/len = 475/STRA = for | | | | | | | |
| 5 | 96850_at | Cluster Incl AI853842 UI-M-BH0-ajq-a-04-0-UI.s1 Mus musculus cDNA, 3 end/clone = UI-M-BH0-ajq-a-04-0-UI/ | P | 243.9 | P | 100 | P | 88.9 | P |
| | | clone_end = 3/gb = AI853642/gi = 5497546/ug = Mm 30036/len = 511/STRA = for | | | | | | | |
| 5 | 98071_f_at | Cluster Incl X77731 M. musculus mRNA for Deoxycytidine kinase/cds = (12,794)/gb = X7731/gi = 456676/ug = Mm 3446/ | P | 469.3 | P | 208.9 | P | 176.1 | A |
| | | len = 2812/STRA = for | | | | | | | |
| 5 | 101498_at | Cluster Incl AF042730 Mus musculus lithium-sensitive myo-inositol monophosphatase A1 (IMPA1) mRNA, complete cds/ | P | 179.4 | P | 90.7 | P | 8.9 | A |
| | | cds = (66,899)/gb = AF042730/gi = 2801800/ug = Mm 8089/len = 2167/STRA = for | | | | | | | |
| 5 | 101996_at | Cluster Incl M80739 1570 bp/cds = UNKNOWN/gb = M80739/gi = 485375/ug = Mm 985/len = 1570/STRA = for | P | 211.1 | P | 146.7 | P | 95.1 | A |
| 5 | 102318_at | Cluster Incl X86000 Mus musculus mRNA for N-glycan alpha 2,8-sialyltransferase/cds = (333,1412)/gb = X86000/ | P | 156.3 | P | 23 | A | 95.6 | P |
| | | gi = 2685331/ug = Mm 10701/len = 6786/STRA = for | | | | | | | |
| 5 | 102321_at | Cluster Incl M93422 Adenylate cyclase 6/cds = (95,3592)/gb = M93422/ | P | 364.6 | P | 166.9 | A | 85.1 | P |
| | | gi = M93422/gi = 191690/ug = Mm 10706/len = 5841/STRA = for | | | | | | | |
| 5 | 104316_at | Cluster Incl AI646098: vw54h09 x1 Mus musculus cDNA, 3 end/clone = IMAGE-1247681/clone_end = 3/gb = AI646098/ | P | 270.1 | P | 128.7 | A | 157.8 | P |
| | | gi = 4724573/ug = Mm 22414/len = 501/STRA = rev | | | | | | | |
| 5 | 94397_at | Cluster Incl AA690091: vr79a09 s1 Mus musculus cDNA, 5 end/clone = IMAGE-1134904/clone_end = 5/gb = AA690091/ | P | 237.8 | P | 81.7 | A | 177.9 | P |
| | | gi = 2691027/ug = Mm 25776/len = 347/STRA = for | | | | | | | |
| 5 | 94979_at | Cluster Incl AW121624: UI-M-BH2 2-acq-g-11-0-UI.s1 Mus musculus cDNA, 3 end/clone = UI-M-BH2.2-aoq-g-11-0-UI/ | P | 156.3 | P | 17.4 | A | 44.2 | P |
| | | clone_end = 3/gb = AW121624/gi = 6096957/ug = Mm 27273/len = 334/STRA = for | | | | | | | |
| 5 | 95001_at | Cluster Incl AB028920 Mus musculus AKAP95 mRNA for A kinase anchor protein 95, complete cds/cds = (38,2101)/ | P | 221.4 | P | 93.4 | A | 92.1 | A |
| | | gb = AB028920/gi = 5931617/ug = Mm 27351/len = 2704/STRA = for | | | | | | | |
| 5 | 95020_at | Cluster Incl AI848868 UI-M-AJ1-ahb-f-03-0-UI s1 Mus musculus cDNA, 3 end/clone = UI-M-AJ1-ahb-f-03-0-UI/ | P | 369.4 | P | 120.2 | P | 163.4 | M |
| | | clone_end = 3/gb = AI848868/gi = 5492774/ug = Mm 27466/len = 496/STRA = for | | | | | | | |
| 5 | 95028_r_at | Cluster Incl C77009 C77009 Mus musculus cDNA, 3 end/clone = J0024D07/clone_end = 3/gb = C77009/gi = 2517339/ | P | 742 | P | 340.1 | P | 262.5 | A |
| | | ug = Mm 27511/len = 496/STRA = rev | | | | | | | |
| 5 | 97919_at | Cluster Incl AI837467 UI-M-AL0-abr-d-06-0-UI s1 Mus musculus cDNA,3 end/clone = UI-M-AL0-abr-d-06-0-UI/ | P | 306.5 | P | 147.3 | P | 69.4 | A |
| | | clone_end = 3/gb = AI837467/gi = 5471680/ug = Mm 30132/len = 228/STRA = for | | | | | | | |
| 5 | 98504_at | Cluster Incl U58513 Mus musculus Rho-associated, coiled-coil forming protein kinase p160 ROCK-2 mRNA, complete cds/ | P | 201.6 | P | 82.5 | A | 82.8 | P |
| | | cds = (0,4166)/gb = U58513/gi = 1514697/ug = Mm 35815/len = 4167/STRA = for | | | | | | | |
| 5 | 100894_at | Cluster Incl AJ007360 Mus musculus mRNA for ORC5-related protein/cds = (65,1372)/gb = AJ007360/gi = 4938333/ | P | 124.9 | P | 72.2 | A | 77.2 | P |
| | | ug = Mm 566/len = 2079/STRA = for | | | | | | | |
| 5 | 103202_at | Cluster Incl AW047476.UI-M-BH1-all-g-04-0-UI s1 Mus musculus cDNA, 3 end/clone = UI-M-BH1-all-g-04-0-UI/ | P | 735.6 | P | 135.1 | A | 179.1 | A |
| | | clone_end = 3/gb = AW047476/gi = 5908005/ug = Mm 1909/len = 248/STRA = for | | | | | | | |
| 5 | 103207_at | Cluster Incl D13543 DNA polymerase alpha 1, 180 kDa/cds = (21,4418)/gb = D13543/gi = 475158/ug = Mm.1923/len = 5323/ | P | 204 | P | 100.6 | M | 102.2 | A |
| | | STRA = for | | | | | | | |
| 5 | 92226_at | Cluster Incl AA866971 vx91g10.r1 Mus musculus cDNA, 5 end/clone = IMAGE-1282626/clone_end = 5/gb = AA866971/ | P | 494.2 | P | 240.9 | P | 259.6 | P |
| | | gi = 2962416/ug = Mm.346/len = 370/STRA = for | | | | | | | |
| 5 | 96526_at | Cluster Incl AW228840 up22f10.x1 Mus musculus cDNA, 3 end/clone = IMAGE-2655115/clone_end = 3/gb = AW228840/ | P | 318 | P | 251.4 | P | 137.3 | P |
| | | gi = 6558136/ug = Mm.2670/len = 133/STRA = for | | | | | | | |
| 5 | 98893_at | Cluster Incl AW048644 UI-M-BH1-amd-d-08-0-UI s1 Mus musculus cDNA, 3 end/clone = UI-M-BH1-amd-d-08-0-UI/ | A | 531.5 | P | 319.8 | A | 170.2 | P |
| | | clone_end = 3/gb = AW048644/gi = 5909173/ug = Mm 28550/len = 430/STRA = for | | | | | | | |
| 5 | 103010_at | Cluster Incl AW048174: UI-M-BH1-amc-b-12-0-UI.s1 Mus musculus cDNA, 3 end/clone = UI-M-BH1-amc-b-12-0-UI/ | P | 323.3 | P | 106.9 | P | 167.2 | A |
| | | clone _end = 3/gb = AW048174/gi = 5908703/ug = Mm.1571/len = 398/STRA = for | | | | | | | |
| 5 | 103076_at | Cluster Incl AW046093 UI-M-BH1-alf-a-06-0-UI s1 Mus musculus cDNA, 3 end/clone = UI-M-BH1-alf-a-06-0-UI/ | P | 737.2 | A | 406.7 | P | 314.3 | P |
| | | clone _end = 3/gb = AW046093/gi = 5906622/ug = Mm 17087/len = 258/STRA = for | | | | | | | |
| 5 | 103422_at | Cluster Incl M63695 CD1d1 antigen/cds = (91,1101)/gb = M63695/gi = 192471/ug = Mm 1894/len = 1838/STRA = for | P | 207.1 | P | 50.1 | A | 21.3 | A |
| 5 | 103895_at | Cluster Incl AW121745: UI-M-BH2 3-aoe-c-07-0-UI s1 Mus musculus cDNA, 3 end/clone = UI-M-BH2 3-aoe-c-07-0-UI/ | A | 309.9 | P | 156 | P | 27.2 | A |
| | | clone _end = 3/gb = AW121745/gi = 6097080/ug = Mm 21299/len = 345/STRA = for | | | | | | | |
| 5 | 104049_at | Cluster Incl AW047134: UI-M-BH1-alu-c-03-0-UI s1 Mus musculus cDNA, 3 end/clone = UI-M-BH1-alu-c-03-0-UI/ | A | 130.1 | P | 84.5 | P | 99.4 | P |
| | | clone _end = 3/gb = AW047134/gi = 5907663/ug = Mm 21510/len = 332/STRA = for | | | | | | | |
| 5 | 94934_at | Cluster Incl U04828: Angiotensin II receptor, type 2/cds = (131,1222)/gb = U04828/gi = 439862/ug = Mm.2679/len = 2827/ | A | 861.7 | P | 119.6 | A | 429.8 | P |
| | | STRA = for | | | | | | | |
| 5 | 96741_at | Cluster Incl AW124129: UI-M-BH2 1-apr-d-08-0-UI s1 Mus musculus cDNA, 3 end/clone = UI-M-BH2 1-apr-d-08-0-UI/ | P | 93.2 | P | 8.4 | M | 14.1 | P |
| | | clone _end = 3/gb = AW124129/gi = 6099659/ug = Mm 28932/len = 192/STRA = rev | | | | | | | |
| 5 | 99999_at | Cluster Incl D83033 Mouse mRNA for nuclear protein, NP220, complete cds/cds = (325,6105)/gb = D83033/gi = 1469808/ | P | 272.3 | P | 159.9 | P | 167.4 | P |
| | | ug = Mm 4503/len = 6345/STRA = for | | | | | | | |
| 5 | 100903_at | Cluster Incl AJ007780 Mus musculus mRNA for poly(ADP-ribose) polymerase-2/cds = (15,1694)/gb = AJ007780/ | A | 275.2 | P | 114.2 | P | 108.9 | A |
| | | gi = 4138232/ug = Mm 5728/len = 1707/STRA = for | | | | | | | |
| 5 | 104267_at | Cluster Incl AI844736: UI-M-AL1-ahq-c-10-0-UI s1 Mus musculus cDNA, 3 end/clone = UI-M-AL1-ahq-c-10-0-UI/ | P | 302.5 | P | 262.4 | P | 202.9 | M |
| | | clone _end = 3/gb = AI844736/gi = 5488642/ug = Mm 28535/len = 406/STRA = rev | | | | | | | |
| 5 | 93887_at | Cluster Incl AI854351 UI-M-BG1-aic-d-05-0-UI s1 Mus musculus cDNA, 3 end/clone = UI-M-BG1-aic-d-05-0-UI/ | A | 252.4 | P | 83 | A | 128.2 | P |
| | | clone _end = 3/gb = AI854351/gi = 5498257/ug = Mm 88860/len = 369/STRA = for | | | | | | | |
| 5 | 94282_at | Cluster Incl AW124297 UI-M-BH2 1-apq-c-02-0-UI s1 Mus musculus cDNA, 3 end/clone = UI-M-BH2 1-apq-c-02-0-UI/ | P | 397.8 | P | 134.7 | P | 248.1 | A |
| | | clone _end = 3/gb = AW124297/gi = 6099827/ug = Mm 22547/len = 237/STRA = for | | | | | | | |
| 5 | 96945_at | Cluster Incl U73143 Mus musculus syndet mRNA, complete cds/cds = (0,632)/gb = U73143/gi = 2065524/ug = Mm.30197/ | P | 147.4 | P | 94.5 | A | 58.9 | A |
| | | len = 633/STRA = for | | | | | | | |
| 5 | 102388_at | Cluster Incl AW121186: UI-M-BH2 3-aok-b-02-0-UI s1 Mus musculus cDNA, 3 end/clone = UI-M-BH2 3-aok-b-02-0-UI/ | P | 173.7 | P | 105.4 | P | 150.9 | P |
| | | clone _end = 3/gb = AW121186/gi = 6096519/ug = Mm 1182/len = 501/STRA = for | | | | | | | |
| 5 | 103813_at | Cluster Incl D85926: Mus musculus mRNA for Sh3yl1, complete cds/cds = (41,1063)/gb = D85926/gi = 1944388/ | P | 70.4 | P | 56 | A | 59.9 | A |
| | | ug = Mm 2090/len = 1226/STRA = for | | | | | | | |
| 5 | 104119_at | Cluster Incl AI845028: UI-M-BGO-ahy-a-01-0-UI s1 Mus musculus cDNA, 3 end/clone = UI-M-BGO-ahy-a-01-0-UI/ | P | 275.3 | P | 48.7 | A | 69.3 | A |
| | | clone_end = 3/gb = AI845028/gi = 5488934/ug = Mm.21977/len = 510/STRA = for | | | | | | | |
| 5 | 104380_at | Cluster Incl Z71268: M musculus mRNA for CMP-stalic acid transporter/cds = (0,1010)/gb = Z71268/gi = 1486407/ | P | 167.8 | P | 128.5 | P | 81.9 | A |
| | | ug = Mm 22684/len = 1185/STRA = for | | | | | | | |
| 5 | 104759_at | Cluster Incl AI851421: UI-M-BH0-akj-c-04-0-UI s1 Mus musculus cDNA, 3 end/clone = UI-M-BH0-akj-c-04-0-UI/ | A | 272.4 | P | 200 | A | 146.6 | M |
| | | clone_end = 3/gb = AI851421/gi = 5495327/ug = Mm 24797/len = 337/STRA = for | | | | | | | |
| 5 | 94386_at | Cluster Incl AI850140 UI-M-BG1-aid-c-11-0-UI s1 Mus musculus cDNA, 3 end/clone = UI-M-BG1-aid-c-11-0-UI/ | A | 413.6 | P | 267.3 | P | 231 | P |
| | | clone_end = 3/gb = AI850140/gi = 5494046/ug = Mm 25622/len = 305/STRA = for | | | | | | | |
| 5 | 95549_at | Cluster Incl D13545 DNA primase, p58 subunit/cds = (144,1661)/gb = D13545/gi = 303662/ug = Mm 27705/len = 1937/ | P | 176.1 | P | 50.5 | A | 71.5 | A |
| | | STRA = for | | | | | | | |
| 5 | 96746_at | Cluster Incl AW124813: UI-M-BH2.1-apk-c-01-0-UI.s1 Mus musculus cDNA, 3 end/clone = UI-M-BH2.1-apk-c-01-0-UI/ | P | 210.7 | P | 159.2 | P | 124.2 | P |
| | | clone_end = 3/gb = AW124813/gi = 6100343/ug = Mm 28953/len = 435/STRA = for | | | | | | | |
| 5 | 96785_at | Cluster Incl AF110520 Mus musculus major histocompatibility complex region NG27, NG28, RPS28, NADH | A | 233.6 | P | 108.7 | A | 137.6 | P |
| | | oxidoreductase NG29, KIFC1, Fas-binding protein, BING1, tapasin, RaIGDS-like, KE2, BING4, beta 1,3-galactosyl | | | | | | | |
| | | transferase, and RPS18 genes, complete cds, S/STRA = for | | | | | | | |
| 5 | 99441_at | Cluster Incl AW123852 UI-M-BH2 1-app-b-06-0-UI.s1 Mus musculus cDNA, 3 end/clone = UI-M-BH2.1-app-b-06-0-UI/ | A | 129.7 | P | 51.2 | P | 35.6 | A |
| | | clone_end = 3/gb = AW123852/gi = 6099382/ug = Mm 40282/len = 451/STRA = for | | | | | | | |
| 5 | 99963_at | Cluster Incl U07861: Zinc finger protein 101/cds = (107,1927)/gb = U07861/gi = 466390/ug = Mm 4417/len = 2177/STRA = rev | P | 117 | P | 24.9 | P | 63 | P |
| 5 | 100880_at | Cluster Incl AA816121 vp44a01.r1 Mus musculus cDNA, 5 end/clone = IMAGE-1079496/clone_end = 5/gb = AA816121/ | P | 726.9 | P | 90 | A | 104.8 | A |
| | | gi = 2885717/ug = Mm.532/len = 316/STRA = rev | | | | | | | |
| 5 | 102957_at | Cluster Incl U20159: Mus musculus 78 kDa tyrosine phosphoprotein SLP-76 mRNA, complete cds/cds = (192,1793)/ | P | 103.8 | P | 76.6 | P | 30.6 | A |
| | | gb = U20159/gi = 806767/ug = Mm 1781/len = 2247/STRA = for | | | | | | | |
| 5 | 103537_at | Cluster Incl AF076681: Mus musculus type-I transmembrane ER-resident serine/threonine kinase PERK mRNA, complete | M | 81.5 | P | 21 | A | 62.8 | P |
| | | cds/cds = (191,3535)/gb = AF076681/gi = 4107512/ug = Mm 23375/len = 4510/STRA = for | | | | | | | |
| 5 | 104670_at | Cluster Incl AI315698 uj34a01 x1 Mus musculus cDNA, 3 end/clone = IMAGE-1921800/clone_end = 3/gb = AI315698/ | P | 214.9 | P | 89 | A | 118.8 | P |
| | | gi = 4030965/ug = Mm 32357/len = 215/STRA = for | | | | | | | |
| 5 | 93241_r_at | Cluster Incl AI838094 UI-M-AL0-abv-g-07-0-UI s1 Mus musculus cDNA, 3 end/clone = UI-M-AL0-abv-g-07-0-UI/ | P | 111.8 | P | 54.5 | A | 54.6 | P |
| | | clone _end = 3/gb = AI838094/gi = 5472307/ug = Mm 5935/len = 344/STRA = rev | | | | | | | |
| 5 | 94088_at | Cluster Incl AW228429 up22e01 y1 Mus musculus cDNA, 5 end/clone = IMAGE-2655096/clone _end = 5/gb = AW228429/ | M | 161.1 | P | 46.8 | A | 101.5 | P |
| | | gi = 6557725/ug = Mm 99371/len = 342/STRA = for | | | | | | | |
| 5 | 95288_i_at | Cluster Incl AA189811 mt97g08.r1 Mus musculus cDNA, 5 end/clone = IMAGE-637886/clone_end = 5/gb = AA189811/ | P | 73.9 | P | 24.4 | A | 38.5 | P |
| | | gi = 1776416/ug = Mm 1882/len = 365/STRA = for | | | | | | | |
| 5 | 95939_i_at | Cluster Incl AW047237: UI-M-BH1-amb-g-05-0-UI.s1 Mus musculus cDNA, 3 end/clone = UI-M-BH1-amb-g-05-0-UI/ | P | 180.7 | P | 103.6 | A | 57.9 | A |
| | | clone_end = 3/gb = AW047237/gi = 5907766/ug = Mm 24851/len = 420/STRA = for | | | | | | | |
| 5 | 93538_at | Cluster Incl AW228036: Up12e07.x1 Mus musculus cDNA, 3 end/clone = IMAGE-2654148/clone_end = 3/gb = AW228036/ | P | 185.9 | P | 38.4 | P | 21.7 | A |
| | | gi = 6557332/ug = Mm 19992/len = 516/STRA = for | | | | | | | |
| 5 | 94812_at | Cluster Incl AA684175 vm68b10. s1 Mus musculus cDNA, 5 end/clone = IMAGE-1003387/clone_end = 5/gb = AA684175/ | P | 238.2 | P | 89.4 | P | 34.2 | A |
| | | gi = 2670761/ug = Mm 22700/len = 513/STRA = for | | | | | | | |
| 5 | 94818_at | Cluster Incl AW047223: UI-M-BH1-amb-e-09-0-UI.s1 Mus musculus cDNA, 3 end/clone = UI-M-BH1-amb-c-09-0-UI/ | P | 597.4 | P | 137.5 | P | 166.6 | M |
| | | clone_end = 3/gb = AW047223/gi = 5907752/ug = Mm 22710/len = 420/STRA = for | | | | | | | |
| 5 | 98048_at | Cluster Incl AF080490 Mus musculus TLS-associated protein TASR-2 mRNA, complete cds/cds = (91,879)/gb = AF060490/ | P | 263.8 | P | 111.6 | P | 86.4 | A |
| | | gi = 3327956/ug = Mm 34279/len = 2693/STRA = for | | | | | | | |
| 5 | 98543_at | Cluster Incl AJ223208: Mus musculus mRNA for cathepsin S, partial/cds = (0,139)/gb = AJ223208/gi = 4138249/ | P | 1174.3 | P | 8 | M | 29.7 | M |
| | | ug = Mm.3619/len = 366/STRA = for | | | | | | | |
| 5 | 99178_at | Cluster Incl AI845652: UI-M-AQ1-ady-b-04-0-UI.a1 Mus musculus cDNA, 3 end/clone = UI-M-AQ1-ady-b-04-0-UI/ | P | 78 | P | 25.1 | A | 22.3 | A |
| | | clone_end = /gb = AI845652/gi = 5489558/ug = Mm.41791/len = 357/STRA = for | | | | | | | |
| 5 | 101082_at | Cluster Incl J02652: Malic enzyme, supernatant/cds = (65,1783)/gb = J02652/gi = 199762/ug = Mm 730/len = 3129/STRA = for | A | 450.1 | P | 330.8 | A | 315.8 | P |
| 5 | 94395_at | Cluster Incl AI194254 ul14a10.r1 Mus musculus cDNA, 5 end/clone = IMAGE-1851258/clone_end = 5/gb = AI194254/ | M | 228 | P | 34 | P | −7.3 | A |
| | | gi = 3745461/ug = Mm 25703/len = 475/STRA = for | | | | | | | |
| 5 | 96231_at | Cluster Incl AW123780 UI-M-BH2 1-apm-c-10-0-UI s1 Mus musculus cDNA, 3 end/clone = UI-M-BH2.1-apm-c-10-0-UI/ | A | 200.8 | A | 146.1 | P | 169.2 | P |
| | | clone_end = 3/gb = AW123780/gi = 6099310/ug = Mm 28775/len = 400/STRA = for | | | | | | | |
| 5 | 96778_at | Cluster Incl AA656775 vr50e03 s1 Mus musculus cDNA, 5 end/clone = IMAGE-1124092/clone_end = 5/gb = AA656775/ | A | 308.8 | P | 181.4 | A | 216.7 | P |
| | | gi = 2592929/ug = Mm 29061/len = 417/STRA = for | | | | | | | |
| 5 | 98026_g_at | Cluster Incl M34896: Ecotropic viral integration site 2/cds = (191,862)/gb = M34896/gi = 193202/ug = Mm.3266/len = 1217/ | P | 324.5 | P | 96 | A | 277.6 | M |
| | | STRA = for | | | | | | | |
| 5 | 98972_at | Cluster Incl AI574262 uj19e05 x1 Mus musculus cDNA, 3 end/clone = IMAGE-1920416/clone_end = 3/gb = AI574262/ | M | 103.2 | P | −121.1 | A | −223.6 | P |
| | | gi = 4537636/ug = Mm 36569/len = 474/STRA = for | | | | | | | |
| 5 | 104263_at | Cluster Incl AA914734 vy91g08.r1 Mus musculus cDNA, 5 end/clone = IMAGE-1313630/clone_end = 5/gb = AA914734/ | A | 170.5 | P | 73.6 | P | 42.5 | A |
| | | gi = 3054126/ug = Mm 28450/len = 470/STRA = rev | | | | | | | |
| 5 | 104274_at | Cluster Incl AW124843 UI-M-BH2.1.apk-f-10-0-UI s1 Mus musculus cDNA, 3 end/clone = UI-M-BH2 1-apk-f-10-0-UI/ | P | 645 | P | 401.7 | P | 399.9 | A |
| | | clone_end = 3/gb = AW124843/gi = 6100373/ug = Mm.28596/len = 509/STRA = for | | | | | | | |
| 5 | 104639_i_at | Cluster Incl AI464596 mz66c12.y1 Mus musculus cDNA, 5 end/clone = IMAGE-718390/clone_end = 5/gb = AI464596/ | P | 301.7 | P | 48.8 | A | 108 | P |
| | | gi = 4318626/ug = Mm 31114/len = 339/STRA = for | | | | | | | |
| 5 | 92724_at | Cluster Incl AI183202 uc50h11.r1 Mus musculus cDNA, 5 end/clone = IMAGE-1429125/clone_end = 5/gb = AI183202/ | P | 380.6 | P | 73 | A | 99.3 | A |
| | | gi = 3733840/ug = Mm.46512/len = 314/STRA = for | | | | | | | |
| 5 | 93858_at | Cluster Incl M33266 Murine macrophage interferon inducible protein 10 (IP-10) mRNA, complete cds/cds = (40,336)/ | P | 285.2 | P | 68.1 | A | 66.2 | A |
| | | gb = M33266/gi = 198444/ug = Mm.877/len = 1063/STRA = rev | | | | | | | |
| 5 | 96090_g_at | Cluster Incl AI255972 ui76a02 x1 Mus musculus cDNA, 3 end/clone = IMAGE-1888298/clone_end = 3/gb = AI255972/ | P | 177.3 | P | 35.2 | A | 38.5 | A |
| | | gi = 3863497/ug = Mm.26726/len = 511/STRA = for | | | | | | | |
| 5 | 101998_at | Cluster Incl AW125086 UI-M-BH2 1-apx-f-03-0-UI s1 Mus musculus cDNA, 3 end/clone = UI-M-BH2 1-apx-f-03-0-UI/ | A | 153.9 | P | 75.3 | A | 45.3 | A |
| | | clone_end = 3/gb = AW125086/gi = 6100616/ug = Mm 9860/len = 461/STRA = for | | | | | | | |
| 5 | 102030_at | Cluster Incl AF026032 X-linked nuclear protein/cds = (220,7650)/gb = AF026032/gi = 3002557/ug = Mm 10141/len = 7673/ | A | 60.2 | P | 18.2 | A | 32.3 | P |
| | | STRA = for | | | | | | | |
| 5 | 102761_at | Cluster Incl AF041060 Mus musculus co-chaperone mt-GrpE#2 mRNA, nuclear gene encoding mitochondrial protein, | P | 202.2 | P | 163.7 | A | 162.7 | P |
| | | partial cds/cds = (0,671)/gb = AF041060/gi = 3411071/ug = Mm 12959/len = 1445/STRA = for | | | | | | | |
| 5 | 103797_at | Cluster Incl AB019388: Cell divison cycle 7-like 1/cds-(86,1780)/gb-AB019388/gi = 3869101/ug = Mm 20842/len = 2708/ | A | 133.9 | P | 52.5 | A | 73.2 | A |
| | | STRA = rev | | | | | | | |
| 5 | 98514_at | Cluster Incl AF004833 Mus musculus tissue factor pathway inhibitor (TFPI) mRNA, complete cds/cds = (104,1024)/ | M | 147.8 | P | 57.9 | P | 67.4 | A |
| | | gb = AF004833/gi = 2897076/ug = Mm 3601/len = 1288/STRA = for | | | | | | | |
| 5 | 99465_at | Cluster Incl AJ132922: Mus musculus mRNA for protein encoded by methyl-CpG-binding protein 2/cds = (35,1489)/ | A | 205.8 | P | 129.5 | A | 168 | A |
| | | gb = AJ132922/gi = 5419961/ug = Mm.40984/len = 10087/STRA = for | | | | | | | |
| 5 | 99507_at | Cluster Incl M21247 Uncoupling protein, mitochondrial/cds = (0,923)/gb = M21247/gi = 202268/ug = Mm 4177/len = 924/ | P | 1688.8 | P | 31.6 | A | 32.2 | A |
| | | STRA = for | | | | | | | |
| 5 | 100431_at | Cluster Incl U42467.Leptin receptor/cds = (60,2744)/gb = U42467/gi = 1139592/ug = Mm 4756/len = 4560/STRA = for | A | 68.2 | P | 37.9 | A | 45.2 | P |
| 5 | 100876_at | Cluster Incl AI841076 UI-M-AH0-acz-a-07-0-UI s1 Mus musculus cDNA, 3 end/clone = UI-M-AH0-acz-a-07-0-UI/ | A | 72.2 | P | −7.3 | M | 14.3 | A |
| | | clone_end = 3/gb = AI841076/gi = 5475289/ug = Mm 5264/len = 424/STRA = for | | | | | | | |
| 5 | 103444_at | Cluster Incl AI272489.uk06e09 y1 Mus musculus cDNA, 5 end/clone = IMAGE-1958632/clone_end = 5/gb = AI272489/ | A | 127.9 | P | 95.2 | A | 79.2 | P |
| | | gi = 3894757/ug = Mm 21492/len = 459/STRA = for | | | | | | | |
| 5 | 103763_at | Cluster Incl AW121855 UI-M-BH2 3-aoi-b-08-0-UI s1 Mus musculus cDNA, 3 end/clone = UI-M-BH2.3-aoi-b-08-0-UI/ | A | 335.2 | P | 171.3 | A | 206.1 | A |
| | | clone_end = 3/gb = AW121855/gi = 6097229/ug = Mm.42637/len = 462/STRA = for | | | | | | | |
| 5 | 104478_at | Cluster Incl U27177 Retinoblastoma-like 1 (p107)/cds = (63,3254)/gb = U27177/gi = 1871224/ug = Mm 2994/len = 4863/ | A | 268.5 | P | 72.7 | P | 5.3 | A |
| | | STRA = for | | | | | | | |
| 6 | 92585_at | Cluster Incl L02526 Protein kinase, mitogen activated, kinase 1, p45/cds = (40,1221)/gb = L02526/gi = 199123/ug = Mm.1059/ | P | 343.3 | P | 172.9 | P | 153.1 | P |
| | | len = 2119/STRA = for | | | | | | | |
| 6 | 92586_at | Cluster Incl X57024: Glutamate dehydrogenase/cds = (76,1752)/gb = X57024/gi = 51081/ug = Mm 10600/len = 2942/ | P | 646.7 | P | 300.6 | P | 522.8 | P |
| | | STRA = for | | | | | | | |
| 6 | 92597_s_at | Cluster Incl U13838 Mus musculus vacuolar adenosine triphosphatase subunit B gene, complete cds/cds = (0,1535)/ | P | 447.7 | P | 176.1 | P | 415.6 | P |
| | | gb = U13838/gi = 1184660/ug = Mm.10727/len = 1536/STRA = for | | | | | | | |
| 6 | 92828_at | Cluster Incl AB004789 Mus musculus mRNA for dolichol-phosphate-mannose synthase, complete cds/cds = (0,782)/ | P | 519.9 | P | 424.5 | P | 451.2 | P |
| | | gb = AB004789/gi = 3073772/ug = Mm.12969/len = 789/STRA = for | | | | | | | |
| 6 | 92847_s_at | Cluster Incl X56831: Mannose-6-phosphate receptor, cation dependent/cds = (98,934)/gb = X56831/gi = 53205/ug = Mm 1358/ | P | 764.2 | P | 673.4 | P | 399.6 | P |
| | | len = 1395/STRA = for | | | | | | | |
| 6 | 92854_at | Cluster Incl D50500 Mouse mRNA for Rab 11, partial sequence/cds = (0,291)/gb = D50500/gi = 1742912/ug = Mm.1387/ | P | 2334.6 | P | 1488.3 | P | 1272.1 | P |
| | | len = 464/STRA = for | | | | | | | |
| 6 | 93037_i_at | Cluster Incl M69260 Lipocortin 1/cds = (14,1054)/gb = M69260/gi = 198844/ug = Mm 14860/len = 1340/STRA = for | P | 1385.4 | P | 1057.7 | P | 1222.7 | P |
| 6 | 93043_at | Cluster Incl D50463 Mouse SDR1 mRNA, complete cds/cds = (27,872)/gb = D50463/gi = 1747303/ug = Mm 15125/len = 1900/ | P | 1001.6 | P | 463.5 | P | 489.9 | P |
| | | STRA = for | | | | | | | |
| 6 | 93066_at | Cluster Incl D16195 Granulin/cds = (0,1769)/gb = D16195/gi = 286067/ug = = Mm 1568/len = 1770/STRA = for | P | 668.8 | P | 308.4 | P | 413.6 | P |
| 6 | 93261_at | Cluster Incl AJ000990 Mus musculus mRNA for legumain/cds = (94,1401)/gb = AJ000990/gi = 3676226/ug = Mm 17185/ | P | 717.4 | P | 263.9 | P | 326 | P |
| | | len = 1889/STRA = for | | | | | | | |
| 6 | 93290_at | Cluster Incl U35374 Mus musculus purine nucleoside phosphorylase (Np-b) mRNA, complete cds/cds = (0,869)/gb = U35374/ | P | 836.7 | P | 326.7 | P | 282.4 | P |
| | | gi = 114566/ug = Mm 17932/len = 870/STRA = for | | | | | | | |
| 6 | 93309_at | Cluster Incl U42386: Fibroblast growth factor Inducible 14/cds = (880,1065)/gb = U42386/gi = 1353710/ug = Mm.18459/ | P | 544 | P | 301.2 | P | 444.4 | P |
| | | len = 2610/STRA = for | | | | | | | |
| 6 | 93320_at | Cluster Incl AF017175: Camitine palmitoyltransferase 1, liver/cds = (0,2296)/gb = AF017175/gi = 2388721/ug = Mm.18522/ | P | 857 | P | 489.9 | P | 554 | P |
| | | len = 4414/STRA = for | | | | | | | |
| 6 | 93347_at | Cluster Incl Z22819: RAB24, member RAS oncogene family/cds = (0,611)/gb = Z22819/gi = 438163/ug = Mm 18800/len = 660/ | P | 541.9 | P | 279.8 | P | 343.7 | P |
| | | STRA = for | | | | | | | |
| 6 | 93364_at | Cluster Incl X59990: Calenin alpha 1/cds = (72,2792)/gb = X59990/gi = 49934/ug = Mm 18962/len = 3713/STRA = for | P | 847.8 | P | 635.8 | P | 409.3 | P |
| 6 | 93497_at | Cluster Incl K02782: Mouse complement component C3 mRNA, alpha and beta subunits, complete cds/cds = (56,5047)/ | P | 880.3 | P | 243.2 | P | 260.4 | P |
| | | gb = K02782/gi = 192278/ug = Mm 19131/len = 5067/STRA = for | | | | | | | |
| 6 | 93498_s_at | Cluster Incl M97216: Mouse DNA-binding protein mRNA, complete cds/cds = (0,1535)/gb = M97216/gi = 515628/ | P | 1503.8 | P | 1372.1 | P | 1250 | P |
| | | ug = Mm 19133/len = 1536/STRA = for | | | | | | | |
| 6 | 93499_at | Cluster Incl U16740: Mus musculus capping protein alpha 1 subunit mRNA, partial cds/cds = (0,856)/gb = U16740/ | P | 1313.3 | P | 911.4 | P | 726.3 | P |
| | | gi = 595916/ug = Mm 19142/len = 2893/STRA = for | | | | | | | |
| 6 | 93818_g_at | Cluster Incl AB025218: Mus musculus mRNA for Sid394p, complete cds/cds = (33,638)/gb = AB025218/gi = 5931556/ | P | 1589.9 | P | 1180.9 | P | 863.6 | P |
| | | ug = Mm 21507/len = 677/STRA = for | | | | | | | |
| 6 | 93819_at | Cluster Incl AI847269: UI-M-AI1-afq-h-06-0-UI.s1 Mus musculus cDNA, 3 end/clone = UI-M-AI1-afq-h-06-0-UI/clone_end = 3/ | P | 2400.8 | P | 1582.8 | P | 2083.7 | P |
| | | gb = AI847269/gi = 5491175/ug = Mm.21507/len = 287/STRA = for | | | | | | | |
| 6 | 93994_at | Cluster Incl AW212131: uo82g04.x1 Mus musculus cDNA, 3 end/clone = IMAGE-2649078/clone_end = 3/gb = AW212131/ | P | 448.5 | P | 223.8 | P | 165.4 | P |
| | | gi = 6518137/ug = Mm 21754/len = 566/STRA = for | | | | | | | |
| 6 | 94002_at | Cluster Incl AI849838: UI-M-AL1-aho-d-08-0-UI.s1 Mus musculus cDNA, 3 end/clone = UI-M-AL1-aho-d-08-0-UI/ | P | 605.7 | P | 308.1 | P | 404.1 | P |
| | | clone_end = 3/gb = AI849838/gi = 5493737/ug = Mm 21771/len = 417/STRA = for | | | | | | | |
| 6 | 94003_at | Cluster Incl AI848510: UI-M-AP1-agf-d-06-0-UI.s2 Mus musculus cDNA, 3 end/clone = UI-M-AP1-agf-d-06-0-UI/ | P | 1107.6 | P | 705.9 | P | 701.7 | P |
| | | clone_end = 3/gb = AI848510/gi = 5492416/ug = Mm 21773/len = 420/STRA = for | | | | | | | |
| 6 | 94041_at | Cluster Incl AI840643: UI-M-AM0-adq-g-08-0-UI.s1 Mus musculus cDNA, 3 end/clone = UI-M-AM0-adq-g-08-0-UI/ | P | 1107.2 | P | 653.8 | P | 690.8 | P |
| | | clone_end = 3/gb = AI840643/gi = 5474851/ug = Mm.21953/len = 517/STRA = for | | | | | | | |
| 6 | 95412_at | Cluster Incl U49112: Mus musculus calcium-binding protein ALG-2 (ALG-257) mRNA, complete cds/cds = (56,631)/ | P | 760.7 | P | 492.5 | P | 521.3 | P |
| | | gb = U49112/gi = 1213519/ug = Mm 24254/len = 1060/STRA = for | | | | | | | |
| 6 | 95428_at | Cluster Incl AA688761: vr07b11.r1 Mus musculus cDNA, 5 end/clone = IMAGE-1111101/clone_end = 5/gb = AA883761/ | P | 316 | P | 189.5 | P | 222.8 | P |
| | | gi = 2678190/ug = Mm 24456/len = 322/STRA = for | | | | | | | |
| 6 | 95434_at | Cluster Incl AI851740: UI-M-BH0-aim-a-03-0-UI.s1 Mus musculus cDNA, 3 end/clone = UI-M-BH0-aim-a-03-0-UI/ | P | 1405.5 | P | 1091.3 | P | 1213.4 | P |
| | | clone_end = 3/gb = AI851740/gi = 5495641/ug = Mm 24498/len = 373/STRA = for | | | | | | | |
| 6 | 95444_at | Cluster Incl AW122274: UI-M-BH2.2-aos-a-04-0-UI.s1 Mus musculus cDNA, 3 end/clone = UI-M-BH2.2-aos-a-04-0-UI/ | P | 1111.6 | P | 815.3 | P | 776.2 | P |
| | | clone_end = 3/gb = AW122274/gi = 6097751/ug = Mm 24584/len = 261/STRA = for | | | | | | | |
| 6 | 95489_at | Cluster Incl AW047953: UI-M-BH1-aln-f-07-0-UI.s1 Mus musculus cDNA, 3 end/clone = UI-M-BH1-aln-f-07-0-UI/ | P | 264.8 | P | 190.3 | P | 221.4 | P |
| | | clone_end = 3/gb = AW047953/gi = 5908482/ug = Mm 25044/len = 331/STRA = for | | | | | | | |
| 6 | 95507_at | Cluster Incl AB025048: Mus musculus mRNA for Sid6061p, complete cds/cds = (69,1025)/gb = AB025048/gi = 5931550/ | P | 235.6 | P | 172.5 | P | 126.9 | P |
| | | ug = Mm 25198/len = 1912/STRA = for | | | | | | | |
| 6 | 96003_at | Cluster Incl AW048332: UI-M-BH1-akk-c-05-0-UI.s1 Mus musculus cDNA, 3 end/clone = UI-M-BH1-akk-c-05-0-UI/ | P | 988.4 | P | 633.9 | P | 773.3 | P |
| | | clone_end = 3/gb = AW048332/gi = 5908861/ug = Mm 25339/len = 333/STRA = for | | | | | | | |
| 6 | 96007_at | Cluster Incl AI835359: UI-M-AQ0-aah-g-05-0-UI s1 Mus musculus cDNA, 3 end/clone = UI-M-AQ0-aah-g-05-0-UI/ | P | 1802.8 | P | 1071.4 | P | 1349.2 | P |
| | | clone_end = 3/gb = AI835359/gi = 5469572/ug = Mm 25393/len = 305/STRA = for | | | | | | | |
| 6 | 96120 at | Cluster Incl AW124750: UI-M-BH2.1-aqa-e-04-0-UI s1 Mus musculus cDNA, 3 end/clone = UI-M-BH2.1-aqa-e-04-0-UI/ | P | 820.3 | P | 542.4 | P | 474.7 | P |
| | | clone_end = 3/gb = AW124750/gi = 6100280/ug = Mm 2701/len = 184/STRA = for | | | | | | | |
| 6 | 96607_at | Cluster Incl AW124902: UI-M-BH2 1-apu-d-12-0-UI s1 Mus musculus cDNA, 3 end/clone = UI-M-BH2.1-apu-d-12-0-UI/ | P | 827 | P | 510.6 | P | 587 | P |
| | | clone_end = 3/gb = AW124902/gi = 6100432/ug = Mm 27065/len = 281/STRA = for | | | | | | | |
| 6 | 96613_at | Cluster Incl AI852095: UI-M-BH0-aja-h-02-0-UI.s1 Mus musculus cDNA, 3 end/clone = UI-M-BH0-aja-h-02-0-UI/ | P | 582.9 | P | 373.3 | P | 366.4 | P |
| | | clone_end = 3/gb = AI852095/gi = 5496001/ug = Mm 27107/len = 381/STRA = for | | | | | | | |
| 6 | 96675_at | Cluster Incl AW124245: UI-M-BH2.1-aph-f-06-0-UI.s1 Mus musculus cDNA, 3 end/clone = UI-M-BH2.1-aph-f-06-0-UI/ | P | 1089.8 | P | 428.1 | P | 583.7 | P |
| | | clone_end = 3/gb = AW124245/gi = 6099775/ug = Mm 27404/len = 375/STRA = for | | | | | | | |
| 6 | 96701_at | Cluster Incl U35833: Mus musculus anthracycline-associated resistance (Arx) mRNA, complete cds/cds = (20,1936)/ | P | 1262.5 | P | 853.1 | P | 675.6 | P |
| | | gb = U35833/gi = 4096673/ug = Mm.27560/len = 2525/STRA = for | | | | | | | |
| 6 | 97262_at | Cluster Incl AW050305: UI-M-BH1-ans-g-11-0-UI.s1 Mus musculus cDNA, 3 end/clone = UI-M-BH1-ans-g-11-0-UI/ | P | 933.6 | P | 689.5 | P | 556.9 | P |
| | | clone_end = 3/gb = AW050305/gi = 5910834/ug = Mm 27899/len = 342/STRA = for | | | | | | | |
| 6 | 97829_at | Cluster Incl AI838053: UI-M-AL0-abv-c-03-0-UI s1 Mus musculus cDNA, 3 end/clone = UI-M-AL0-abv-c-03-0-UI/ | P | 224.5 | P | 80.2 | P | 204 | P |
| | | clone_end = 3/gb = AI836053/gi = 5472266/ug = Mm 28219/len = 367/STRA = for | | | | | | | |
| 6 | 97844_at | Cluster Incl U67187: Mus musculus G protein signaling regulator RGS2 (rgs2) mRNA, complete cds/cds = (20,655)/ | P | 414.4 | P | 120 | P | 138 | P |
| | | gb = U67187/gi = 1911236/ug = Mm 28262/len = 1240/STRA = for | | | | | | | |
| 6 | 97904_at | Cluster Incl AW 123953: UI-M-BH2.3-aqc-d-06-0-UI.s1 Mus musculus cDNA, 3 end/clone = UI-M-BH2.3-aqc-d-06-0-UI/ | P | 2161 | P | 1769.9 | P | 1727.1 | P |
| | | clone_end = 3/gb = AW123953/gi = 6099483/ug = Mm.28464/len = 382/STRA = for | | | | | | | |
| 6 | 94452_g_at | Cluster Incl AI787627: ul20f06.y1 Mus musculus cDNA, 5 end/clone = IMAGE-2088131/clone_end = 5/gb = AI787627/ | P | 796.6 | P | 648.7 | P | 653.5 | P |
| | | gi = 5335343/ug = Mm 28786/len = 450/STRA = for | | | | | | | |
| 6 | 94461_at | Cluster Incl AI852144: UI-M-BH0-ajb-e-09-0-UI.s1 Mus musculus cDNA, 3 end/clone = UI-M-BH0-ajb-e-09-0-UI/ | P | 218.9 | P | 117.2 | P | 147.5 | P |
| | | clone_end = 3/gb = AI652144/gi = 5496055/ug = Mm 28830/len = 425/STRA = for | | | | | | | |
| 6 | 94502_at | Cluster Incl AW125724: UI-M-BH2 2-aql-d-12-0-UI.s1 Mus musculus cDNA, 3 end/clone = UI-M-BH2.2-aql-d-12-0-UI/ | P | 206.4 | P | 123.5 | P | 138.6 | P |
| | | clone_end = 3/gb = AW125724/gi = 8101294/ug = Mm 2895/len = 460/STRA = for | | | | | | | |
| 6 | 95070_at | Cluster Incl AW125874: UI-M-BH2 2-aqk-f-09-0-UI s1 Mus musculus cDNA, 3 end/clone = UI-M-BH2.2-aqk-f-09-0-UI/ | P | 370.6 | P | 281.4 | P | 248.1 | P |
| | | clone_end = 3/gb = AW125874/gi = 6101404/ug = Mm 29192/len = 266/STRA = for | | | | | | | |
| 6 | 95086_at | Cluster Incl AI835392: UI-M-AQ0-aaj-b-10-0-UI.s1 Mus musculus cDNA, 3 end/clone = UI-M-AQ0-aaj-b-10-0-UI/ | P | 927.3 | P | 785.6 | P | 886.8 | P |
| | | clone_end = 3/gb = AI835392/gi = 5469605/ug = Mm 29259/len = 221/STRA = for | | | | | | | |
| 6 | 95140_at | Cluster Incl AI842068: UI-M-AN1-afg-b-01-0-UI.s1 Mus musculus cDNA, 3 end/clone = UI-M-AN1-afg-b-01-0-UI/ | P | 805.7 | P | 320.1 | P | 430.3 | P |
| | | clone_end = 3/gb = AI842068/gi = 5476281/ug = Mm 29436/len = 397/STRA = for | | | | | | | |
| 6 | 95148_at | Cluster Incl AB020202: Mus musculus mRNA for adenylate kinase isozyme 2, complete cds/cds = (42,740)/gb = AB020202/ | P | 534.1 | P | 229.7 | P | 176.8 | P |
| | | gi = 4760597/ug = Mm.29460/len = 983/STRA = for | | | | | | | |
| 6 | 95654_at | Cluster Incl AF109905: Mus musculus major histocompatibility locus class III regions Hsc70t gene, partial cds; smRNP, | P | 1533.8 | P | 954 | P | 965.8 | P |
| | | G7A, NG23, MutS homolog, CLCP, NG24, NG25, and NG26 genes, complete cds, and unknown genes/cds = (0,725)/ | | | | | | | |
| | | gb = AF109905/gi = 3986751/ug = Mm 29/STRA = for | | | | | | | |
| 6 | 95708_at | Cluster Incl AI843466 UI-M-AQ1-aec-g-01-0-UI s1 Mus musculus cDNA, 3 end/clone = UI-M-AQ1-aoc-g-01-0-UI/ | P | 1329.9 | P | 970.3 | P | 772.6 | P |
| | | clone_end = 3/gb = AI843466/gi = 5477684/ug = Mm 29702/len = 325/STRA = for | | | | | | | |
| 6 | 96281_at | Cluster Incl AW121246 UI-M-BH2 3-aok-g-05-0-UI s1 Mus musculus cDNA, 3 end/clone = UI-M-BH2 3-aok-g-05-0-UI/ | P | 2317.9 | P | 2270.4 | P | 1903 | P |
| | | clone_end = 3/gb = AW121246/gi = 6096579/ug = Mm 29868/len = 355/STRA = for | | | | | | | |
| 6 | 96333_g_at | Cluster Incl AW259199.um91a03 y1 Mus musculus cDNA, 5 end/clone = lMAGE-2332108/clone_end = 5/gb = AW259199/ | P | 541.4 | P | 183.6 | P | 450 | P |
| | | gi = 6632180/ug = Mm 29971/len = 411/STRA = for | | | | | | | |
| 6 | 96348_at | Cluster Incl AW121217.UI-M-BH2 3-aok-d-10-0-UI s1 Mus musculus cDNA, 3 end/clone = UI-M-BH2 3-aok-d-10-0-UI/ | P | 1376.2 | P | 1084.3 | P | 1111.2 | P |
| | | clone_end = 3/gb = AW121217/gi = 6096550/ug = Mm 29998/len = 454/STRA = for | | | | | | | |
| 6 | 96353_at | Cluster Incl AW125346 UI-M-BH2 1-apy-h-02-0-UI s1 Mus musculus cDNA, 3 end/clone = UI-M-BH2 1-apy-h-02-0-UI/ | P | 574.4 | P | 407.5 | P | 342.4 | P |
| | | clone_end = 3/gb = AW125346/gi = 6100876/ug = Mm 30005/len = 382/STRA = for | | | | | | | |
| 6 | 96913_at | Cluster Incl AW122615 UI-M-BH2.2 aol-b-06-0-UI s1 Mus musculus cDNA, 3 end/clone = UI-M-BH2 2-aol-b-06-0-UI/ | P | 1762.3 | P | 790.8 | P | 943.1 | P |
| | | clone_end = 3/gb = AW1226l5/gi = 6098142/ug = Mm.30147/len = 244/STRA = for | | | | | | | |
| 6 | 96936_at | Cluster Incl AI020792 ub04g10 r1 Mus musculus cDNA, 5 end/clone = IMAGE-1366050/clone_end = 5/gb = AI020792/ | P | 378.3 | P | 315.6 | P | 252.3 | P |
| | | gi = 3235128/ug = Mm 30183/len = 421/STRA = for | | | | | | | |
| 6 | 97486_at | Cluster Incl AA693246 vr58e09.s1 Mus musculus cDNA, 5 end/clone = lMAGE-1124872/clone_end = 5/gb = AA693246/ | P | 916.9 | P | 771.7 | P | 783.2 | P |
| | | gi = 2694184/ug = Mm 30926/len = 397/STRA = for | | | | | | | |
| 6 | 98101_at | Cluster Incl AI851798 UI-M-BH0-ais-a-06-0-UI.s1 Mus musculus cDNA, 3 end/clone = UI-M-BH0-als-a-06-0-UI/ | P | 333.9 | P | 246.9 | P | 272.4 | P |
| | | clone_end = 3/gb = AI851798/gi = 5495704/ug = Mm.34772/len = 300/STRA = for | | | | | | | |
| 6 | 98102_at | Cluster Incl M76727: Pyruvate dehydrogenase E1 alpha subunit/cds = (111,1283)/gb = M76727/gi = 2002768/ug = Mm 34775/ | P | 1486.4 | P | 926.8 | P | 1080 | P |
| | | len = 1446/STRA = for | | | | | | | |
| 6 | 98521_at | Cluster Incl AI847972 UI-M-AI1-all-h-04-0-UI s1 Mus musculus cDNA, 3 end/clone = UI-M-AI1-all-h-04-0-UI/clone_end = 3/ | P | 734.2 | P | 613.3 | P | 473.1 | P |
| | | gb = AI847972/gi = 5491878/ug = Mm.35808/len = 172/STRA = for | | | | | | | |
| 6 | 98525_f_at | Cluster Incl AJ007909.Mus musculus mRNA for erythroid differentiation regulator, partial/cds = (154,687)/gb = AJ007909/ | P | 1740.1 | P | 1266.2 | P | 930.7 | P |
| | | gi = 3336885/ug = Mm.35829/len = 715/STRA = for | | | | | | | |
| 6 | 99158_at | Cluster Incl U58888 Mus musculus SH3-containing protein SH3P2 mRNA, partial cds/cds = (0,755)/gb = U58888/ | P | 717.4 | P | 490.9 | P | 474.2 | P |
| | | gi = 1407662/ug = Mm 4165/len = 1041/STRA = for | | | | | | | |
| 6 | 99169_at | Cluster Incl AW122165 UI-M-BH2.2-aor-g-07-0-UI s1 Mus musculus cDNA, 3 end/clone = UI-M-BH2.2-aor-g-07-0-UI/ | P | 679.4 | P | 692.1 | P | 517.3 | P |
| | | clone_end = 3/gb = AW122165/gi = 6097606/ug = Mm.41705/len = 204/STRA = for | | | | | | | |
| 6 | 99587_at | Cluster Incl X89650 RAB7, member RAS oncogene family/cds = (156,779)/gb = X89650/gi = 1050550/ug = Mm.4268/ | P | 1376.7 | P | 1255.6 | P | 952.8 | P |
| | | len = 2089/STRA = for | | | | | | | |
| 6 | 99610_at | Cluster Incl X93357 Synovial sarcoma, translocated to X Chromosome/cds = (179,1435)/gb = X93357/gi = 1072383/ | P | 774.9 | P | 423.1 | P | 429.7 | P |
| | | ug = Mm 4296/len = 3107/STRA = for | | | | | | | |
| 6 | 100572_at | Cluster Incl AI851289 UI-M-BH0-akf-f-05-0-UI s1 Mus musculus cDNA, 3 end/clone = UI-M-BH0-akf-f-05-0-UI/ | P | 596.6 | P | 475 | P | 320.6 | P |
| | | clone_end = 3/gb = AI851289/gi = 5495195/ug = Mm.5885/len = 449/STRA = for | | | | | | | |
| 6 | 100595_at | Cluster Incl AF035644: Mus musculus potentially prenylated protein tyrosine phosphatase mPRL-2 (Prl2) mRNA, complete | P | 1547 | P | 1305.6 | P | 1170.2 | P |
| | | cds/cds = (179,682)/gb = AF035644/gi = 2992629/ug = Mm 6355/len = 723/STRA = for | | | | | | | |
| 6 | 100603_at | Cluster Incl AF063231: Dynein, cytoplasmic, intermediate chain 2/cds = (77,1915)/gb = AF063231/gi = 3493590/ | P | 230.4 | P | 143.2 | P | 137.6 | P |
| | | ug = Mm 6441/len = 2458/STRA = for | | | | | | | |
| 6 | 101040_at | Cluster Incl D38117: Mus musculus mRNA for m-calpain large subunit, complete cds/cds = (170,2272)/gb = D38117/ | P | 916.7 | P | 531.6 | P | 741.5 | P |
| | | gi = 2570157/ug = Mm 6958/len = 3225/STRA = for | | | | | | | |
| 6 | 101982_at | Cluster Incl X98475 M musculus VASP gene/cds = (0,1130)/gb = X98475/gi = 1617401/ug = Mm.9684/len = 1131/STRA = for | P | 809 | P | 583.5 | P | 591.5 | P |
| 6 | 102407_at | Cluster Incl M68898: Mast cell protease 5/cds = (5,748)/gb = M68898/gi = 199028/ug = Mm 1252/len = 960/STRA = for | P | 3412.2 | P | 94 | P | 210.6 | P |
| 6 | 102790_at | Cluster Incl AB016490 Mus musculus mJTB mRNA, complete cds/cds = (306,746)/gb = AB016490/gi = 3721891/ | P | 1336.7 | P | 965.8 | P | 948 | P |
| | | ug = Mm 13912/len = 1273/STRA = for | | | | | | | |
| 6 | 103018_at | Cluster Incl AF096868 Mus musculus transcytosis associated protein p115 mRNA, partial cds/cds = (0,2827)/gb = AF096868/ | P | 302.4 | P | 165.7 | P | 184.2 | P |
| | | gi = 3860050/ug = Mm 15868/len = 3690/STRA = for | | | | | | | |
| 6 | 103899_at | Cluster Incl AA690863 vt32b05 r1 Mus musculus cDNA, 5 end/clone = IMAGE-1164753/clone_end = 5/gb = AA690863/ | P | 380.9 | P | 218.1 | P | 215.9 | P |
| | | gi = 2691799/ug = Mm 21313/len = 289/STRA = for | | | | | | | |
| 6 | 104315_at | Cluster Incl AI846773 UI-M-AO1-ael-f-02-0-UI s1 Mus musculus cDNA, 3 end/clone = UI-M-AO1-ael-f-02-0-UI/ | P | 364.1 | P | 234 | P | 357.3 | P |
| | | clone_end = 3/gb = AI846773/gi = 5490679/ug = Mm.22413/len = 322/STRA = for | | | | | | | |
| 6 | 104350_at | Cluster Incl AI050321 ub29c11 r1 Mus musculus cDNA, 5 end/clone = IMAGE-1379158/clone_end = 5/gb = AI050321/ | P | 366.2 | P | 250.9 | P | 255 | P |
| | | gi = 3299438/ug = Mm.22550/len = 504/STRA = for | | | | | | | |
| 6 | 104612_g_at | Cluster Incl AI854008 UI-M-BH0-aiv-h-12-0-UI s1 Mus musculus cDNA, 3 end/clone = UI-M-BH0-aiv-h-12-0-UI/ | P | 298.2 | P | 215.3 | P | 239.4 | P |
| | | clone_end = 3/gb = AI854008/gi = 5497914/ug = Mm.24158/len = 494/STRA = for | | | | | | | |
| 6 | 94369_at | Cluster Incl AW123026 UI-M-BH2 1-apb-f-07-0-UI s1 Mus musculus cDNA, 3 end/clone = UI-M-BH2 1-apb-f-07-0-UI/ | P | 236.4 | P | 191.8 | P | 66.8 | P |
| | | clone_end = 3/gb = AW123026/gi = 6098556/ug = Mm.25336/len = 442/STRA = for | | | | | | | |
| 6 | 94408_at | Cluster Incl U47008: Ngfi-A binding protein 1/cds = (136,1596)/gb = U47008/gi = 1197670/ug = Mm 25903/len = 1796/ | P | 1379.9 | P | 861.4 | P | 1012.6 | P |
| | | STRA = for | | | | | | | |
| 6 | 94427_at | Cluster Incl AI841737 UI-M-AL0-abo-g-05-0-UI.s1 Mus musculus cDNA, 3 end/clone = UI-M-AL0-abo-g-05-0-UI/ | P | 251 | P | 197.6 | P | 212.8 | P |
| | | clone_end = 3/gb = AI841737/gi = 5475950/ug = Mm.26422/len = 372/STRA = for | | | | | | | |
| 6 | 94963_at | Cluster Incl AI462105 ub70b01 x1 Mus musculus cDNA, 3 end/clone = IMAGE-1383049/clone_end = 3/gb = AI462105/ | P | 1267 | P | 767.7 | P | 867.2 | P |
| | | gi = 4316135/ug = Mm.27206/len = 422/STRA = for | | | | | | | |
| 6 | 95608_at | Cluster Incl AI851255 UI-M-BH0-akf-c-03-0-UI.s1 Mus musculus cDNA, 3 end/clone = UI-M-BH0-akf-c-03-0-UI/ | P | 249.9 | P | 66.9 | P | 111.6 | P |
| | | clone_end = 3/gb = AI851255/gi = 5495161/ug = Mm.27986/len = 427/STRA = rev | | | | | | | |
| 6 | 96239_at | Cluster Incl AA815503 vp20d05.r1 Mus musculus cDNA, 5 end/clone = IMAGE-1069161/clone_end = 5/gb = AA815503/ | P | 230.5 | P | 188.8 | P | 162 | P |
| | | gi = 2885099/ug = Mm.28816/len = 477/STRA = for | | | | | | | |
| 6 | 97939_at | Cluster Incl AW125079: UI-M-BH2.1-apx-e-08-0-UI.s1 Mus musculus cDNA, 3 end/clone = UI-M-BH2.1-apx-e-08-0-UI/ | P | 862.9 | P | 631.8 | P | 563.6 | P |
| | | clone_end = 3/gb = AW125079/gi = 6100609/ug = Mm.30265/len = 316/STRA = for | | | | | | | |
| 6 | 97949_at | Cluster Incl M16238: Fibrinogen-like protein 2/cds = (60,1358)/gb = M16238/gi = 193305/ug = Mm 3040/len = 3772/STRA = for | P | 486.4 | P | 202.8 | P | 223.1 | P |
| 6 | 98466_r_at | Cluster Incl M31419 Interferon activated gene 204/cds = (127,2049)/gb = M31419/gi = 198399/ug = Mm 34817/len = 2353/ | P | 421.1 | P | 106.2 | P | 254.6 | P |
| | | STRA = for | | | | | | | |
| 6 | 99051_at | Cluster Incl M36579 S100 calcium-binding protein A4/cds = (0,164)/gb = M36579/gi = 199816/ug = Mm 3925/len = 302/ | P | 818.1 | P | 376.2 | P | 349.6 | P |
| | | STRA = for | | | | | | | |
| 6 | 99503_at | Cluster Incl AW045204 UI-M-BH1-alh-c-10-0-UI s1 Mus musculus cDNA, 3 end/clone = UI-M-BH1-alh-c-10-0-UI/ | P | 286.6 | P | 149.1 | P | 202 | P |
| | | clone_end = 3/gb = AW045204/gi = 5905733/ug = Mm.41714/len = 405/STRA = for | | | | | | | |
| 6 | 99996_at | Cluster Incl Y07941: M musculus mRNA for plakophilin 1/cds = (12,2198)/gb = Y07941/gi = 1707593/ug = Mm 4494/len = 4504/ | P | 2099.3 | P | 1026.5 | P | 1156.5 | P |
| | | STRA = rev | | | | | | | |
| 6 | 101867_at | Cluster Incl U11680 Mus musculus glycerol-3-phosphate acyltransferase gene, nuclear gene encoding mitochondrial/ | P | 729.4 | P | 502.7 | P | 682.2 | P |
| | | protein, 5 region and partial cds/cds = (411,425)/gb = U11680/gi = 786006/ug = Mm 87773/len = 426/STRA = rev | | | | | | | |
| 6 | 101886_f_at | Cluster Incl X52490 Synthetic D(b) mRNA for D(b) glycoprotein (alpha chain) and beta(2) microglobulin/cds = (9,1097)/ | P | 2015 | P | 940.2 | P | 1219 | P |
| | | gb = X52490/gi = 50671/ug = Mm.88493/len = 1102/STRA = rev | | | | | | | |
| 6 | 101948_at | Cluster Incl X05212 Mouse mRNA for laminin B1 C-terminus/cds = (0,1489)/gb = X05212/gi = 52861/ug = Mm.963/len = 1640/ | P | 773 | P | 440.9 | P | 329.8 | P |
| | | STRA = for | | | | | | | |
| 6 | 102658_at | Cluster Incl X59769 Interleukin 1 receptor, type II/cds = (92,1324)/gb = X59769/gi = 52670/ug = Mm 1349/len = 1341/ | P | 385.8 | P | 136 .2 | P | 368.2 | P |
| | | STRA = for | | | | | | | |
| 6 | 103471_at | Cluster Incl AI194333 ui15d12 r1 Mus musculus cDNA, 5 end/clone = IMAGE-1851383/clone_end = 5/gb = AI194333/ | P | 304.9 | P | 147.7 | P | 171.5 | P |
| | | gi = 3745540/ug = Mm 21821/len = 459/STRA = for | | | | | | | |
| 6 | 103744_at | Cluster Incl AI852760 UI-M-BH0-ary-b-12-0-UI s1 Mus musculus cDNA,3 end/clone = UI-M-BH0-ary-b-12-0-UI/ | P | 90.3 | P | 53.3 | P | 25.5 | P |
| | | clone_end = 3/gb = AI852760/gi = 5496666/ug = Mm 24472/len = 417/STRA = for | | | | | | | |
| 6 | 104249_g_at | Cluster Incl AW227650 up 12b09 y1 Mus musculus cDNA, 5 end/clone = IMAGE-2654105/clone_end = 5/gb = AW227650/ | P | 417.2 | P | 193.5 | P | 354.8 | P |
| | | gi = 6556946/ug = Mm.28345/len = 440/STRA = for | | | | | | | |
| 6 | 93721_at | Cluster Incl L12367 Mus musculus adenylyl cyclase-associated protein (CAP) mRNA, complete cds/cds = (64,1488)/ | P | 333.3 | P | 253 | P | 244.9 | P |
| | | gb = L12367/gi = 293316/ug = Mm 8687/len = 1563/STRA = for | | | | | | | |
| 6 | 93871_at | Cluster Incl L32838 Mouse germline interleukin 1 receptor antagonist (IL-1m) gene,complete cds/cds = (0,536)/gb = L32838/ | P | 377.8 | P | 203.3 | P | 103.1 | P |
| | | gi = 487864/ug = Mm.882/len = 537/STRA = for | | | | | | | |
| 6 | 99325_at | Cluster Incl M32502. Wingless-related MMTV integration site 3/cds = (45,1112)/gb = M32502/gi = 198428/ug= Mm 5188/ | P | 270 | P | 108.2 | P | 240.8 | P |
| | | len = 3000/STRA = for | | | | | | | |
| 6 | 101741_at | Cluster Incl AB003305: Proteasome beta type subunit 5, pseudogene/cds = (0,797)/gb = AB003305/gi = 2897807/ | P | 998.5 | P | 641 | P | 493.9 | P |
| | | ug = Mm 57067/len = 798/STRA = for | | | | | | | |
| 6 | 97593_f_at | Cluster Incl C79694 C79694 Mus musculus cDNA, 3 end/clone = J0070F03/clone_end = 3/gb = C79694/gi = 2520024/ | P | 522.1 | P | 395.3 | P | 454.3 | P |
| | | ug = Mm 89577/len = 439/STRA = rev | | | | | | | |
| 6 | 101697_f_at | Cluster Incl AI844607 UI-M-AJ1-ahh-d-12-0-UI s2 Mus musculus cDNA, 3 end/clone = UI-M-AJ1-ahh-d-12-0-UI/ | P | 326.9 | P | 179.1 | P | 202.9 | P |
| | | clone_end = 3/gb = AI844607/gi = 5488513/ug = Mm.39586/len = 121/STRA = rev | | | | | | | |
| 6 | 94084_at | Cluster Incl AI842888 UI-M-AN1-afb-f-12-0-UI s1 Mus musculus cDNA, 3 end/clone = UI-M-AN1-afb-f-12-0-UI/ | P | 286.1 | P | 163.5 | P | 115.8 | M |
| | | clone_end = 3/gb = AI842888/gi = 5477101/ug = Mm 22192/len = 331/STRA = for | | | | | | | |
| 6 | 96103_f_at | Cluster Incl X92411: RAD23b homolog (S cerevisiae)/cds = (0,1250)/gb = X92411/gi = 1044898/ug = Mm 26879/len = 1251/ | P | 289 | P | 135.5 | P | 117.6 | P |
| | | STRA = for | | | | | | | |
| 6 | 97480_f_at | Cluster Incl U95607 Mus musculus testis specific DNA-homolog mRNA, complete cds/cds = (72,800)/gb = U95607/ | P | 50.2 | P | 40.3 | P | 22.7 | A |
| | | gi = 2286122/ug = Mm 3075/len = 1030/STRA = for | | | | | | | |
| 6 | 98099_at | Cluster Incl AW045760 UI-M-BH1-akt-b-12-0-UI.s1 Mus musculus cDNA, 3 end/clone = UI-M-BH1-akt-b-12-0-UI/ | P | 217.4 | P | 121.4 | A | 148.9 | P |
| | | clone_end = 3/gb = AW045760/gi = 5906289/ug = Mm. 34750/len = 413/STRA = for | | | | | | | |
| 6 | 100059_at | Cluster Incl M31775 Mouse cytochrome beta-558 mRNA, 3 end/cds = (0,574)/gb = M31775/gi = 192913/ug = Mm 448/ | P | 877.5 | P | 549.8 | P | 692.2 | P |
| | | len = 677/STRA = for | | | | | | | |
| 6 | 101106_at | Cluster Incl AI853331.UI-M-BH0-air-a-07-0-UI s1 Mus musculus cDNA, 3 end/clone = UI-M-BH0-air-a-07-0-UI/ | P | 294.1 | P | 164.5 | P | 181.1 | P |
| | | clone_end = 3/gb = AI853331/gi = 5497237/ug = Mm.7514/len = 347/STRA = for | | | | | | | |
| 6 | 102012_at | Cluster Incl AB014485 Mus musculus mRNA for RA70, complete cds/cds = (65,1141)/gb = AB014485/gi = 4730810/ | P | 584.6 | P | 217.2 | P | 167.5 | A |
| | | ug = Mm 10019/len = 1554/STRA = for | | | | | | | |
| 6 | 102813_f_at | Cluster Incl Aw210346 ul53g12 y1 Mus musculus cDNA, 5 end/clone = IMAGE-2102086/clone_end = 5/gb = AW210346/ | P | 268.2 | P | 153.7 | P | 125.5 | P |
| | | gi = 6516286/ug = Mm 14253/len = 431/STRA = for | | | | | | | |
| 6 | 103347_at | Cluster Incl AI852548 UI-M-BH0-arw-f-09-0-UI s1 Mus musculus cDNA, 3 end/clone = UI-M-BH0-alw-f-09-0-UI/ | P | 323.7 | P | 155.8 | P | 33.7 | P |
| | | clone_end = 3/gb = AI852548/gi = 5496454/ug = Mm.1828/len = 350/STRA = for | | | | | | | |
| 6 | 103905_at | Cluster Incl AI314958 uj34107.x1 Mus musculus cDNA, 3 end/clone = IMAGE-1921861/clone_end = 3/gb = AI314958/ | P | 303.2 | P | 118.6 | A | 124.9 | P |
| | | gi = 4030225/ug = Mm.21397/len = 487/STRA = rev | | | | | | | |
| 6 | 104154_at | Cluster Incl AB021961 Mus musculus mutant p53 mRNA, complete cds/cds = (100,1272)/gb = AB021961/gi = 5421849/ | P | 427.8 | P | 310.7 | P | 167.4 | A |
| | | ug = Mm 222/len = 1429/STRA = for | | | | | | | |
| 6 | 104761_at | Cluster Incl AA612450: vo70h07 r1 Mus musculus cDNA, 5 end/clone = IMAGE-1064509/clone_end = 5/gb = AA612450/ | P | 277.9 | P | 89.1 | A | 137.4 | P |
| | | gi = 2461128/ug = Mm.24842/len = 294/STRA = for | | | | | | | |
| 6 | 94390_at | Cluster Incl AI646981 ub68c08 x1 Mus musculus cDNA, 3 end/clone = IMAGE-1382894/clone_end = 3/gb = AI646981/ | P | 231.2 | P | 152.2 | P | 146.8 | A |
| | | gi = 4725456/ug = Mm.25645/len = 540/STRA = for | | | | | | | |
| 6 | 94931_at | Cluster Incl AI851104 UI-M-BH0-akg-c-11-0-UI s1 Mus musculus cDNA, 3 end/clone = UI-M-BH0-akg-c-11-0-UI/ | P | 326.3 | P | 184.5 | P | 159.4 | P |
| | | clone_end = 3/gb = AI851104/gi = 5495010/ug = Mm 26697/len = 378/STRA = for | | | | | | | |
| 6 | 95592_at | Cluster Incl AW046785 UI-M-BH1-alz-a-05-0-UI s1 Mus musculus cDNA, 3 end/clone = UI-M-BH1-alz-a-05-0-UI/ | P | 249.8 | P | 230.5 | P | 140.7 | A |
| | | clone_end = 3/gb = AW046785/gi = 5907314/ug = Mm 279/len = 454/STRA = for | | | | | | | |
| 6 | 96812_at | Cluster Incl AF089721: Smoothened homolog (Drosophila)/cds = UNKNOWN/gb = AF089721/gi = 3608491/ug = Mm 29279/ | P | 211.2 | P | 176.8 | P | −13.3 | A |
| | | len = 1073/STRA = for | | | | | | | |
| 6 | 99019_at | Cluster Incl D17571. P450 (cytochrome) oxidoreductase/cds = (52,2088)/gb = D17571/gi = 407342/ug = Mm 3863/len = 2457/ | P | 387 | P | 366.4 | P | 215.4 | A |
| | | STRA = rev | | | | | | | |
| 6 | 103441 _at | Cluster Incl AI194248: ui14a03.r1 Mus musculus cDNA, 5 end/clone = IMAGE-1851244/clone_end = 5/gb = AI194248/ | P | 182.2 | P | 41.9 | P | 127.3 | P |
| | | gi = 3745455/ug = Mm.21431/len = 438/STRA = for | | | | | | | |
| 6 | 92481_at | Cluster Incl AF086905: Mus musculus protein kinase Chk2 (Chk2) mRNA, complete cds/cds = (0,1640)/gb = AF086905/ | P | 230.9 | P | 111.3 | P | 27.2 | A |
| | | gi = 3982841/ug = Mm 42149/len = 1821/STRA = for | | | | | | | |
| 6 | 97197_r_at | Cluster Incl C78850 C78850 Mus musculus cDNA, 3 end/clone = J0056C12/clone_end = 3/gb = C78850/gi = 2519180/ | P | 534.4 | P | 264.5 | P | 102.4 | A |
| | | ug = Mm 36896/len = 219/STRA = rev | | | | | | | |
| 6 | 99378_f_at | Cluster Incl M18837: Mouse MHC class I Q4 beta-2-microglobulin (Qb-1) gene, complete cds/cds = (0,980)/gb = M18837/ | P | 521 | P | 266.2 | A | 279.1 | P |
| | | gi = 199323/ug = Mm 56926/len = 981/STRA = for | | | | | | | |
| 6 | 101816_at | Cluster Incl AF063937 Squamous cell carcinoma antigen 2/cds = (72,1238)/gb = AF063937/gi = 3885487/ug = Mm 57172/ | P | 577.9 | P | 236.6 | P | 150.2 | P |
| | | len = 1239/STRA = for | | | | | | | |
| 6 | 102161_f_at | Cluster Incl X58609 Mouse MHC (Qa) Q2-k gene for class I antigen, exons 1-3/cds = (0,1088)/gb = X58609/gi = 53854/ | P | 709.9 | P | 426.4 | P | 295.7 | P |
| | | ug = Mm 46812/len = 1089/STRA = for | | | | | | | |
| 6 | 93017_at | Cluster Incl AF077527 Mus musculus syntenin mRNA, complete cds/cds = (54,950)/gb = AF077527/gi = 3342559/ | P | 112.7 | P | 39.9 | A | 48.6 | A |
| | | ug = Mm 14744/len = 2043/STRA = for | | | | | | | |
| 6 | 93590_at | Cluster Incl AI844370 UI-M-AL1-ahp-b-12-0-UI s1 Mus musculus cDNA, 3 end/clone = UI-M-AL1-ahp-b-12-0-UI/ | P | 241 | P | 91.5 | P | 91.6 | P |
| | | clone_end = 3/gb = AI844370/gi = 5488276/ug = Mm 20818/len = 430/STRA = for | | | | | | | |
| 6 | 93775_at | Cluster Incl AI841894 UI-M-AO0-acd-d-09-0-UI s1 Mus musculus cDNA, 3 end/clone = UI-M-AO0-acd-d-09-0-UI/ | P | 428.5 | P | 193.3 | P | −13.9 | A |
| | | clone_end = 3/gb = AI841894/gi = 5476107/ug = Mm.2121/len = 435/STRA = for | | | | | | | |
| 6 | 94298_at | Cluster Incl X53619 M musculus P35B gene, exon 1/cds = (0,815)/gb = X53619/gi = 53556/ug = Mm 22596/len = 816/ | P | 282.6 | P | 270.6 | P | 185.6 | P |
| | | STRA = for | | | | | | | |
| 6 | 97876_at | Cluster Incl AI842008 UI-M-AH0-ada-c-09-0-UI.s1 Mus musculus cDNA, 3 end/clone = UI-M-AH0-ada-c-09-0-UI/ | P | 581.4 | P | 431 | P | 323 | A |
| | | clone_end = 3/gb = AI842008/gi = 5476221/ug = Mm.28356/len = 431/STRA = for | | | | | | | |
| 6 | 97515_at | Cluster Incl X89998 M musculus mRNA for 17 beta-hydroxysteroid dehydrogenase type IV/cds = (12,2219)/gb = X89998/ | P | 311.9 | P | 99.1 | P | 132.9 | A |
| | | gi = 1213007/ug = Mm 3195/len = 2468/STRA = for | | | | | | | |
| 6 | 97541_f_at | Cluster Incl X00246 Mouse mRNA with a Set 1 repetitive element for a class I major histocompatibility complex(MHC) | P | 440.9 | P | 39.2 | P | −82 | A |
| | | antigen/cds = (0,857)/gb = X00246/gi = 53085/ug = Mm.33263/len = 1484/STRA = rev | | | | | | | |
| 6 | 98088_at | Cluster Incl X13333 CD14 antigen/cds = (32,1132)/gb = X13333/gi = 50338/ug = Mm.3460/len = 1366/STRA = for | P | 1250 | P | 533.1 | P | 581.2 | A |
| 6 | 98092_at | Cluster Incl AA790307 vw17a10 r1 Mus musculus cDNA, 5 end/clone = IMAGE-1244058/clone_end = 5/gb = AA790307/ | P | 1018.5 | P | 235.9 | P | 45.1 | A |
| | | gi = 2850427/ug = Mm.34609/len = 581/STRA = for | | | | | | | |
| 6 | 98589_at | Cluster Incl M93275 Adipose differentiation related protein/cds = (78,1335)/gb = M93275/gi = 191692/ug = Mm 381/len = 1680/ | P | 1360.2 | P | 952.5 | P | 811.6 | P |
| | | STRA = for | | | | | | | |
| 6 | 101540_at | Cluster Incl AF069519: Mus musculus T-G mismatch-specific thymine-DNA glycosylase TDGb isoform mRNA, complete | P | 574 | P | 368.4 | P | 312.6 | A |
| | | cds/cds = (108,1301)/gb = AF069519/gi = 3420292/ug = Mm 88099/len = 2859/STRA = for | | | | | | | |
| 6 | 101566_f_at | Cluster Incl M17818 Mouse major urinary prolein group 1 gene, 5 end, clone BL6-3/cds = (66,95)/gb = M17818/gi = 202305/ | P | 2632.4 | P | 5.2 | A | −12.3 | A |
| | | ug = Mm 89481/len = 96/STRA = for | | | | | | | |
| 6 | 103808_at | Cluster Incl AB016784 Mus musculus ref-11 mRNA, complete cds/cds = (261,2450)/gb = AB016784/gi = 3551508/ | P | 366.7 | P | 240.7 | A | 237.6 | M |
| | | ug = Mm 20885/len = 2829/STRA = for | | | | | | | |
| 6 | 98410_at | Cluster Incl AJ007972 Mus musculus mRNA for GTPI protein/cds = (16,1203)/gb = AJ1007972)/gi = 4158175/ug = Mm 33902/ | P | 383.1 | P | 127.1 | A | 203 | P |
| | | len = 3338/STRA = for | | | | | | | |
| 6 | 101465_at | Cluster Incl U06924 Signal transducer and activator of transcription 1/cds = (4,2253)/gb = U06924/gi = 458709/ug = Mm 8249/ | P | 183.2 | P | 32.8 | P | 33 | A |
| | | len = 2277/STRA = for | | | | | | | |
| 6 | 101923_at | Cluster Incl U34277 Mus musculus PAF acetylhydrolase mRNA, complete cds/cds = (116,1438)/gb = U34277/gi = 1041235/ | P | 584.4 | P | 205.2 | P | 177.3 | A |
| | | ug = Mm 9277/len = 1507/STRA = for | | | | | | | |
| 6 | 102641_at | Cluster Incl L03215: SFFV proviral integration 1/cds = UNKNOWN/gb = L03215/gi = 201035/ug = Mm.1302/len = 1232/ | P | 368.6 | P | 258.4 | P | 220.3 | M |
| | | STRA = for | | | | | | | |
| 6 | 102706_l_at | Cluster Incl X61597 M musculus gene for kallikrein-binding protein/cds = (111,1364)/gb = X61597/gi = 52808/ug = Mm 14191/ | P | 67.5 | P | 37.6 | P | 67.5 | P |
| | | len = 1690/STRA = for | | | | | | | |
| 6 | 102707_f_at | Cluster Incl X61597 M musculus gene for kallikrein-binding protein/cds = (111,1364)/gb = X61597/gi = 52808/ug = Mm 14191/ | P | 233.6 | P | 115.6 | P | 73.8 | A |
| | | len = 1690/STRA = for | | | | | | | |
| 6 | 104202_at | Cluster Incl AI850624 UI-M-BG1-alk-d-02-0-UI s1 Mus musculus cDNA, 3 end/clone = UI-M-BG1-alk-d-02-0-UI/ | P | 343.5 | P | 168.6 | P | 242.6 | P |
| | | clone_end = 3/gb = AI850624/gi = 5494530/ug = Mm 27539/len = 416/STRA = rev | | | | | | | |
| 6 | 104456_at | Cluster Incl AA863742 vx08b05 r1 Mus musculus cDNA, 5 end/clone = IMAGE-1263825/clone_end = 5/gb = AA863742/ | P | 104.7 | P | 62.3 | A | 81.4 | A |
| | | gi = 2956221/ug = Mm 29514/len = 402/STRA = for | | | | | | | |
| 6 | 104677_at | Cluster Incl AI852008: UI-M-BH0-aiq-f-08-0-UI.s1 Mus musculus cDNA, 3 end/clone = UI-M-BH0-aiq-f-08-0-UI/ | P | 180.8 | P | 81.9 | A | 23.8 | M |
| | | clone_end = 3/gb = AI852008/gi = 5495914/ug = Mm 32542/len = 410/STRA = for | | | | | | | |
| 6 | 92251_f_at | Cluster Incl AA960657 ub60c10 s1 Mus musculus cDNA, 3 end/clone = IMAGE-1382130/clone_end = 3/gb = AA960657/ | P | 391.1 | P | 47.9 | A | 119.1 | A |
| | | gi = 3126557/ug = Mm 35110/len = 467/STRA = rev | | | | | | | |
| 6 | 93339_at | Cluster Incl AI846243 UI-M-AK1-aez-a-10-0-UI s1 Mus musculus cDNA, 3 end/clone = UI-M-AK1-aez-a-10-0-UI/ | P | 629.9 | P | 160.4 | A | 295.2 | M |
| | | clone_end = 3/gb = AI846243/gi = 5490149/ug = Mm 18706/len = 334/STRA = for | | | | | | | |
| 6 | 99663_g_at | Cluster Incl AI194767: ul56d10 x1 Mus musculus cDNA, 3 end/clone = IMAGE-1886419/clone_end = 3/gb = AI194767/ | P | 393 | P | 370.8 | P | 131.1 | A |
| | | gi = 3747373/ug = Mm 4379/len = 525/STRA = for | | | | | | | |
| 6 | 101588_at | Cluster Incl AF058055: Mus musculus monocarboxylate transpoter 1 (mMCT1) mRNA, complete cds/cds = (189,1670)/ | P | 60.9 | P | 13.7 | A | 30 | P |
| | | gb = AF058055/gi = 3047399/ug = Mm 9086/len = 3184/STRA = for | | | | | | | |
| 6 | 102027_s_at | Cluster Incl AA204010 mu59g02 r1 Mus musculus cDNA, 5 end/clone = IMAGE-643730/clone_end = 5/gb = AA204010/ | A | 317 | P | −49.1 | A | 66.9 | P |
| | | gi = 1800701/ug = Mm 10124/len = 304/STRA = for | | | | | | | |
| 6 | 103016_s_at | Cluster Incl X68273: CD68 antigen/cds = (91,1071)/gb = X68273/gi = 52988/ug = Mm 15819/len = 1234/STRA = for | P | 375 | P | 29.7 | A | 66.6 | A |
| 6 | 103397_at | Cluster Incl AA795486 vq95e08 r1 Mus musculus cDNA, 5 end/clone = IMAGE-1110086/clone_end = 5/gb = AA795486/ | P | 126.9 | P | 60.5 | A | 88.3 | P |
| | | gi = 2858441/ug = Mm 18713/len = 273/STRA = for | | | | | | | |
| 6 | 95543_at | Cluster Incl AI843046 UI-M-AK1-aeq-g-04-0-UI s1 Mus musculus cDNA, 3 end/clone = UI-M-AK1-aeq-g-04-0-UI/ | P | 313.2 | A | 191.7 | A | 275.5 | A |
| | | clone_end = 3/gb = AI843046/gi = 5477259/ug = Mm 27685/len = 432/STRA = rev | | | | | | | |
| 6 | 97346_at | Cluster Incl AW124115: UI-M-BH2.1-apr-b-11-0-UI s1 Mus musculus cDNA, 3 end/clone = UI-M-BH2.1-apr-b-11-0-UI/ | P | 337.4 | P | 92.5 | A | 76.7 | A |
| | | clone_end = 3/gb = AW124115/gi = $$/ug = Mm $$/len = $$/STRA = for | | | | | | | |
| 6 | 98428_at | Cluster Incl AJ246002: Mus musculus mRNA for spastin protein orthologue (Spast gene)/cds = (0,1516)/gb = AJ246002/ | P | 54.6 | P | 12.2 | P | 18.5 | A |
| | | gi = 6273571/ug = Mm 34234/len = 1689/STRA = for | | | | | | | |
| 6 | 101160_at | Cluster Incl X53798: Macrophage inflammatory protein 2/cds = (46,348)/gb = X53798/gi = 53128/ug = Mm 4979/len = 1083/ | P | 330.3 | P | 79.1 | A | 43.2 | A |
| | | STRA = for | | | | | | | |
| 6 | 96031_r_at | Cluster Incl AW259411. up29c03.x1 Mus musculus cDNA, 3 end/clone = IMAGE-2655748/clone_end = 3/gb = AW259411/ | P | 118.6 | P | 29.6 | A | 56.1 | A |
| | | gi = 6632465/ug = Mm 25794/len = 465/STRA = for | | | | | | | |
| 6 | 95752_at | Cluster Incl AI837369.UI-M-AP0-abm-a-02-0-UI s1 Mus musculus cDNA, 3 end/clone = UI-M-AP0-abm-a-02-0-UI/ | P | 502.7 | P | 324 | A | 263.4 | M |
| | | clone_end = 3/gb = AI837369/gi = 5471582/ug = Mm 29784/len = 371/STRA = for | | | | | | | |
| 6 | 97507_at | Cluster Incl X67809 Peptidylprotyl isomerase C-associated protein/cds = (168,1901)/gb = X67809/gi = 297032/ug = Mm 3152/ | P | 406.4 | P | −5.1 | A | 25.3 | A |
| | | len = 2168/STRA = for | | | | | | | |
| 6 | 102313_at | Cluster Incl L09737 GTP cyclohydrolase 1/cds = (122,847)/gb = L09737/gi = 293356/ug = Mm. 10651/len = 994/STRA = for | P | 99 | P | 17.8 | A | 37 | M |
| 6 | 103805_g_at | Cluster Incl AW231026: uo70a01.y1 Mus musculus cDNA, 5 end/clone = IMAGE-2647848/clone_end = 5/gb = AW231026/ | P | 235.1 | P | 132.8 | A | 150 | A |
| | | gi = 6580258/ug = Mm 20156/len = 482/STRA = for | | | | | | | |
| 6 | 103887_at | Cluster Incl M83219 Mus musculus intracellular calcium-binding protein (MRP14) mRNA, complete cds/cds = (33,374)/ | P | 1908.7 | P | −22.4 | A | −62.9 | A |
| | | gb = M83219/gi = 199807/ug = Mm 2128/len = 488/STRA = for | | | | | | | |
| 6 | 104528_at | Cluster Incl AI181549: uc59h04 r1 Mus musculus cDNA, 5 end/clone = IMAGE-1429975/clone_end = 5/gb = AI181549/ | P | 128.4 | P | −18.3 | P | −78.8 | A |
| | | gi = 3732187/ug = Mm 2310/len = 435/STRA = for | | | | | | | |
| 6 | 104549_at | Cluster Incl AI844761: UI-M-AL1-ahq-e-12-0-UI s1 Mus musculus cDNA, 3 end/clone = UI-M-AL1-ahq-e-12-0-UI/ | P | 281.8 | P | 132.3 | A | 121.7 | P |
| | | clone_end = 3/gb = AI844761/gi = 5488667/ug = Mm 23702/len = 384/STRA = for | | | | | | | |
| 6 | 104606_at | Cluster Incl M55561 CD80 antigen/cds = (78,302)/gb = M55561/gi = 200225/ug = Mm 24130/len = 491/STRA = for | P | 712.9 | P | 292.6 | A | 512.8 | A |
| 6 | 96188_at | Cluster Incl AF052506 Mus musculus double-stranded RNA-specific adenosine deaminase mRNA, complete cds/ | P | 177.8 | A | 61.7 | A | 118.4 | P |
| | | cds = (6,3484)/gb = AF052506/gi = 2981096/ug = Mm 28502/len = 3466/STRA = for | | | | | | | |
| 6 | 97409_at | Cluster Incl U19119 Mus musculus G-protein-like LRG-47 mRNA, complete cds/cds = (445,1674)/gb = U19119/gi = 633753/ | P | 253.4 | P | −7.3 | A | 10.6 | A |
| | | ug = Mm 29938/len = 2227/STRA = for | | | | | | | |
| 6 | 98500_at | Cluster Incl D13695 Lymphocyte antigen 84/cds = (42,1745)/gb = D13695/gi = 286100/ug = Mm 35692/len = 4989/STRA = for | P | 134.4 | P | 32.1 | A | 38.9 | A |
| 6 | 103295_at | Cluster Incl AI854482 UI-M-BH0-ait-h-07-0-UI s1 Mus musculus cDNA, 3 end/clone = UI-M-BH0-ait-h-07-0-UI/clone_end = 3/ | P | 332.1 | A | 254.3 | P | 213.6 | P |
| | | gb = AI854482/gi = 5498388/ug = Mm 20971/len = 389/STRA = for | | | | | | | |
| 6 | 101681_f_at | Cluster Incl U21906 Histocompatibility 2, blastocyst/cds = (236,1285)/gb = U21906/gi = 2326165/ug = Mm 34289/len = 1515/ | P | 696.4 | P | 460.8 | P | 377.8 | A |
| | | STRA = for | | | | | | | |
| 6 | 92811_at | Cluster Incl X74154 Retinol binding protein 2, cellular/cds = (57,461)/gb = X74154/gi = 397351/ug = Mm 12825/len = 479/ | P | 199.2 | P | 36.5 | A | 30 | A |
| | | STRA = for | | | | | | | |
| 6 | 95505_at | Cluster Incl AW060509 UI-M-BH1-ann-c-12-0-UI.s1 Mus musculus cDNA, 3 end/clone = UI-M-BH1-ann-c-12-0-UI/ | P | 81.9 | P | 47.2 | P | −20.6 | A |
| | | clone_end = 3/gb = AW060509/gi = 6008260/ug = Mm.25187/len = 308/STRA = for | | | | | | | |
| 6 | 98045_s_at | Cluster Incl U18869: Mus musculus mitogen-responsive 96 kDa phosphoprotein p96 mRNA, alternatively spliced p67 | P | 579.3 | P | −38.5 | A | 12.7 | A |
| | | mRNA, and alternatively spliced p93 mRNA, complete cds/cds = (216,2516)/gb = U18869/gi = 1176369/ug = Mm 34248/ | | | | | | | |
| | | len = 3347/STRA = for | | | | | | | |
| 6 | 98562_at | Cluster Incl X58861: Complement component 1, q subcomponent, alpha polypeptide/cds = (29,766)/gb = X58861/gi = 50226/ | P | 526.4 | P | −73.6 | A | 22.9 | A |
| | | ug = Mm.370/len = 1033/STRA = for | | | | | | | |
| 6 | 100611_at | Cluster Incl M21050 Mouse lysozyme M gene/cds = (30,476)/gb = M21050/gi = 198950/ug = Mm 654/len = 1264/STRA = for | P | 465.3 | P | −75.8 | A | −36.9 | A |
| 6 | 102351_at | Cluster Incl J05118: Carboxypeptidase A3, mast cell/cds = (6,1259)/gb = J05118/gi = 192372/ug = Mm 1135/len = 1442/ | P | 1294.4 | P | 3.2 | A | 68 | A |
| | | STRA = rev | | | | | | | |
| 6 | 102791_at | Cluster Incl U22033 Large multifunctional protease 7/cds = (0,830)/gb = U22033/gi = 972985/ug = Mm 13913/len = 831/ | P | 362.9 | P | 74.2 | A | 58.6 | A |
| | | STRA = for | | | | | | | |
| 6 | 104728_at | Cluster Incl L27439 Mus musculus protein S mRNA, partial cds/cds = (0,1933)/gb = L27439/gi = 487866/ug = Mm 24611/ | P | 285.5 | P | 49.6 | A | 42 | A |
| | | len = 3079/STRA = for | | | | | | | |
| 6 | 98465_f_at | Cluster Incl M31419 Interferon activated gene 204/cds = (127,2049)/gb = M31419/gi = 198399/ug = Mm 34817/len = 2353/ | P | 596.9 | P | −31.5 | A | −5 | A |
| | | STRA = for | | | | | | | |
| 6 | 100397_at | Cluster Incl AF024637 TYRO protein tyrosine kinase binding protein/cds = (0,344)/gb = AF024637/gi = 2906001/ | P | 595.6 | P | 151.7 | A | 206.1 | A |
| | | ug = Mm 46301/len = 345/STRA = for | | | | | | | |
| 6 | 102264_at | Cluster Incl AF099972 Mus musculus schlafen1 (Slfn1) mRNA, complete cds/cds = (257,1270)/gb = AF099972/gi = 3983147/ | P | 300.2 | P | 211.6 | A | 256.3 | A |
| | | ug = Mm 10948/len = 1881/STRA = for | | | | | | | |
| 6 | 103210_at | Cluster Incl M29855 Interleukin 3 receptor, beta chain 2/cds = (175,2811)/gb = M29855/gi = 198342/ug = Mm 1940/len = 4613/ | P | 133.2 | P | 96.4 | P | 30.4 | A |
| | | STRA = for | | | | | | | |
| 6 | 103226_at | Cluster Incl Z11974 Mannose receptor, C type 1/cds = (99,4469)/gb = Z11974/gi = 52997/ug = Mm 2019/len = 5085/ | P | 516.3 | P | −14.5 | A | −12.2 | A |
| | | STRA = for | | | | | | | |
| 6 | 103707_at | Cluster Incl U77461 Complement component 3a receptor 1/cds = (0,1433)/gb = U77461/gi = 2130534/ug = Mm 2408/ | P | 202.2 | P | 184.7 | A | 81.3 | A |
| | | len = 1434/STRA = for | | | | | | | |
| 6 | 92223_at | Cluster Incl X66295: Complement component 1, q subcomponent, c polypeptide/cds = (38,778)/gb = X66295/gi = 50228/ | P | 495.7 | P | 11.1 | A | −62.8 | A |
| | | ug = Mm.3453/len = 1019/STRA = for | | | | | | | |
| 6 | 93622_at | Cluster Incl M57626 Mast cell protease 6/cds = (33,863)/gb = M57626/gi = 200508/ug = Mm.7409/len = 1108/STRA = for | P | 1929.7 | P | 284.3 | M | 383 | M |
| 6 | 99413_at | Cluster Incl U29678 Chemokine (C-C) receptor 1/cds = (0,1067)/gb = U29678/gi = 1109785/ug = Mm 57051/len = 1068/ | P | 169.8 | P | 17.3 | A | −16.3 | A |
| | | STRA = for | | | | | | | |
| 6 | 100328_s_at | Cluster Incl U96684 Paired-lg-like receptor A3/cds = (10,2001)/gb = U96684/gi = 2138356/ug = Mm 87801/len = 2101/ | P | 305.2 | P | 100.4 | A | 158.4 | A |
| | | STRA = for | | | | | | | |
| 6 | 95806_f_at | Cluster Incl M68899 Mouse mast cell chymase 2 mRNA, complete cds/cds = (120,650)/gb = M68899/gi = 199030/ | P | 530.4 | P | 71.1 | A | 96.1 | A |
| | | ug = Mm 87865/len = 901/STRA = for | | | | | | | |
| 6 | 102104_f_at | Cluster Incl AI504305 vl04g11 x1 Mus musculus cDNA, 3 end/clone = IMAGE-963236/clone_end = 3/gb = AI504305/ | P | 562.1 | P | −15.5 | A | 16.7 | A |
| | | gi = 4402156/ug = Mm.42485/len = 580/STRA = rev | | | | | | | |
| 6 | 95159_at | Cluster Incl AI846849: UI-M-AI1-afk-e-04-0-UI.s1 Mus musculus cDNA, 3 end/clone = UI-M-AI1-afk-o-04-0-UI/clone_end = 3/ | P | 105.3 | P | −54.1 | M | 41.4 | A |
| | | gb = AI846849/gi = 5490755/ug = Mm.29484/len = 332/STRA = for | | | | | | | |
| 6 | 96297_at | Cluster Incl AI845934: UI-M-AK1-aex-f-03-0-UI.s1 Mus musculus cDNA, 3 end/clone = UI-M-AK1-aex-f-03-0-UI/ | P | 138.3 | P | −11.6 | A | −47.8 | A |
| | | clone_end = 3/gb = AI845934/gi = 5489840/ug = Mm.29906/len = 280/STRA = for | | | | | | | |
| 6 | 97530_at | Cluster Incl U82627: Mus musculus ubiquitin-conjugating enzyme mE2 mRNA, complete cds/cds = (105,581)/gb = U82627/ | P | 210 | P | 73.4 | A | 96.3 | A |
| | | gi = 1857450/ug = Mm 3268/len = 769/STRA = for | | | | | | | |
| 6 | 100100_at | Cluster Incl AA762212-vv47h06 r1 Mus musculus cDNA, 5 end/clone = IMAGE-1225595/clone_end = 5/gb = AA762212/ | P | 67.6 | P | 26.3 | A | −9.5 | A |
| | | gi = 2811959/ug = Mm 46316/len = 509/STRA = for | | | | | | | |
| 6 | 102021_at | Cluster Incl M27960 Mouse interleukin-4 receptor (secreted form) mRNA, complete cds/cds = (236,928)/gb = M27960/ | P | 169.2 | P | 1.4 | A | −52.2 | A |
| | | gi = 198365/ug = Mm 1009/len = 3697/STRA = for | | | | | | | |
| 6 | 102353_at | Cluster Incl M31039 Mus musculus complement receptor 3 beta subunit MAC-1/mRNA, complete cds/cds = (0,2315)/ | P | 61 | P | 19.4 | A | −4.1 | A |
| | | gb = M31039/gi = 198434/ug = Mm 1137/len = 2459/STRA = for | | | | | | | |
| 6 | 103394_at | Cluster Incl U72680 Mus musculus ion channel homolog RIC mRNA, complete cds/cds = (48,584)/gb = U72680/gi = 1872490/ | P | 340 | P | 126.4 | A | −7.4 | A |
| | | ug = Mm 1870/len = 797/STRA = for | | | | | | | |
| 6 | 96772_at | Cluster Incl J04620 Mouse primase p49 subunit (prlA) mRNA, complete cds/cds = (73,1326)/gb = J04620/gi = 200196/ | A | 213.5 | P | 121.4 | A | 147.6 | A |
| | | ug = Mm 2903/len = 1507/STRA = for | | | | | | | |
| 6 | 99979_at | Cluster Incl X78445 M musculus Cyp1-b-1 mRNA for cytochrome P450/cds = (179,1810)/gb = X78445/gi = 506463/ | P | 35.1 | P | −98 | A | −99.9 | A |
| | | ug = Mm 4443/len = 2006/STRA = for | | | | | | | |
| 6 | 101392_at | Cluster Incl AW122839 UI-M-BH2 1-aoz-e-09-0-UI.s1 Mus musculus cDNA, 3 end/clone = UI-M-BH2.1-aoz-e-09-0-UI/ | P | 168.9 | P | 104.5 | A | 124.4 | A |
| | | clone_end = 3/gb = AW122839/gi = 6098369/ug = Mm 7196/len = 382/STRA = for | | | | | | | |
| 6 | 102292_at | Cluster Incl U00937: Mus musculus GADD45 protein (gadd45) gene, complete cds/cds = (0,497)/gb = U00937/gi = 392933/ | A | 168.9 | P | −13.2 | P | −7.4 | M |
| | | ug = Mm 1236/len = 498/STRA = for | | | | | | | |
| 6 | 104006_at | Cluster Incl L21768 Mus musculus eps15 mRNA, complete cds/cds = (110,2803)/gb = L21768/gi = 404756/ug = Mm 267/ | P | 132.8 | P | 28.1 | A | 97.4 | P |
| | | len = 3033/STRA = for | | | | | | | |
| 6 | 104657_at | Cluster Incl D26177.Leukemia Inhibitory factor receptor/cds = (71,3349)/gb = D26177/gi = 473718/ug = Mm.3174/len = 3399/ | P | 73.7 | P | 21.4 | A | 71.2 | A |
| | | STRA = for | | | | | | | |
| 6 | 92443_I_at | Cluster Incl X16493 Zinc finger protein 1/cds = (320,1594)/gb = X16493/gi = 55482/ug = Mm 4184/len = 1939/STRA = rev | A | 102.6 | A | −18.4 | A | −43.1 | A |
| 6 | 92472_f_at | Cluster Incl AF099973 Mus musculus schlafen2 (Slfn2) mRNA, complete cds/cds = (182,1318)/gb = AF099973/gi = 3983149/ | P | 247.7 | P | 44.9 | A | 58.6 | A |
| | | ug = Mm 42124/len = 1684/STRA = for | | | | | | | |
| 6 | 92718_at | Cluster Incl AI158810 ud38d09 r1 Mus musculus cDNA, 5 end/clone = IMAGE-1448177/clone_end = 5/gb = AI158810/ | P | 468.7 | P | 39.2 | A | 18.2 | A |
| | | gi = 3691992/ug = Mm 48382/len = 251/STRA = for | | | | | | | |
| 6 | 94774_at | Cluster Incl M31416 Interferon activated gene 202/cds = (293,1830)/gb = M31418/gi = 198396/ug = Mm.16744/len = 1857/ | P | 548 | P | −3.5 | A | 3.3 | A |
| | | STRA = for | | | | | | | |
| 6 | 98822_at | Cluster Incl X56602 Mus musculus mRNA Interferon-induced 15-KDa protein/cds = (111,593)/gb = X58602/gi = 52737/ | P | 1077.4 | P | 12 | A | −158.5 | A |
| | | ug = Mm 4950/len = 728/STRA = for | | | | | | | |
| 6 | 97013_f_at | Cluster Incl AW046124 UI-M-BH1-alf-e-03-0-UI s1 Mus musculus cDNA, 3 end/clone = UI-M-BH1-alf-e-03-0-Ul/ | P | 360.2 | P | 94.2 | A | 268.6 | A |
| | | clone_end = 3/gb = AW046124/gi = 5906653/ug = Mm.88950/len = 249/STRA = rev | | | | | | | |
| 6 | 101635_f_at | Cluster Incl M16360 Mouse major unnary protein V (MUP V) mRNA, complete cds/cds = (58,600)/gb = M16360/gi = 199884/ | M | 845.2 | P | −21.8 | A | −20.7 | A |
| | | ug = Mm 30274/len = 872/STRA = for | | | | | | | |
| 6 | 101682_f_at | Cluster Incl M16358 Major unnary protein 4/cds = (66,602)/gb = M16358/gi = 199882/ug = Mm 34335/len = 875/STRA = for | A | 573.3 | P | 64 | A | 41.8 | A |
| 7 | 92539_at | Cluster Incl M16465 Calpactin I light chain/cds = (68,361)/gb = M16465/gi = 192360/ug = Mm.1/len = 600/STRA = for | P | 2730.6 | P | 2577.7 | P | 3448.1 | P |
| 7 | 92603_at | Cluster Incl U13840: Mus musculus vacuolar adenosine triphosphatase subunit D mRNA, complete cds/cds = (17,1072)/ | P | 943.3 | P | 970.8 | P | 804.2 | P |
| | | gb = U13840/gi = 3955099/ug = Mm 1081/len = 1509/STRA = for | | | | | | | |
| 7 | 92607_at | Cluster Incl AF017994.Mus musculus Peg1/MEST protein (Peg1/MEST) gene, partial cds/cds = (247,427)/gb = AF017994/ | P | 1598.5 | P | 843.1 | P | 960.8 | P |
| | | gi = 2570405/ug = Mm.1089/len = 428/STRA = for | | | | | | | |
| 7 | 92638_at | Cluster Incl Z67745 M. musculus mRNA for phosphatase 2A catalytic subunit, Isotype alpha/cds = (0,929)/gb = Z67745/ | P | 385.2 | P | 364.6 | P | 390.5 | P |
| | | gi = 1246794/ug = Mm.11711/len = 930/STRA = for | | | | | | | |
| 7 | 92855_at | Cluster Incl Z50159 M musculus mRNA for Sui1/cds = (0,230)/gb = Z50159/gi = 927434/ug = Mm 13886/len = 355/STRA = for | P | 1259.2 | P | 1068.6 | P | 1279.5 | P |
| 7 | 93093_at | Cluster Incl U35623.Myeloid cell leukemia sequence 1/cds = (48,1043)/gb = U35623/gi = 2522268/ug = Mm 1639/len = 3396/ | P | 1114.3 | P | 960.6 | P | 1148.3 | P |
| | | STRA = for | | | | | | | |
| 7 | 93100_at | Cluster Incl X13297 Mouse mRNA for vascular smooth muscle alpha-actin/cds = (49,1182)/gb = X13297/gi = 49861/ | P | 944.4 | P | 823.5 | P | 576.7 | P |
| | | ug = Mm 16537/len = 1340/STRA = for | | | | | | | |
| 7 | 93327_at | Cluster Incl AI842665 UI-M-AO1-aeo-a-02-0-UI.s1 Mus musculus cDNA, 3 end/clone = UI-M-AO1-aeo-a-02-0-UI/ | P | 208 | P | 128.9 | P | 148.6 | P |
| | | clone_end = 3/gb = AI842685/gi = 5476878/ug = Mm 186/len = 288/STRA = for | | | | | | | |
| 7 | 93353_at | Cluster Incl AF013262: Lumican/cds = (109,1125)/gb = AF013262/gi = 2654773/ug = Mm 18888/len = 1782/STRA = for | P | 2545.2 | P | 1189.3 | P | 1295.2 | P |
| 7 | 93517_at | Cluster Incl Z18272 Procollagen, type VI, alpha 2/cds = (0,1115)/gb = Z18272/gi = 57957/ug = Mm.1949/len = 1562/ | P | 2155.3 | P | 1848.5 | P | 2161.6 | P |
| | | STRA = for | | | | | | | |
| 7 | 93743_at | Cluster Incl AI838388: UI-M-AJO-aav-f-02-0-UI.s2 Mus musculus cDNA, 3 end/clone = UI-M-AJ0-aav-f-02-0-UI/ | P | 260.7 | P | 68 | P | 109.3 | P |
| | | clone_end = 3/gb = AI838388/gi = 5472601/ug = Mm.21069/len = 414/STRA = for | | | | | | | |
| 7 | 93793_at | Cluster Incl AW122780 UI-M-BH2.1-aoy-c-12-0-UI s1 Mus musculus cDNA, 3 end/clone = UI-M-BH2 1-aoy-c-12-0-UI/ | P | 869.5 | P | 837 | P | 869.7 | P |
| | | clone_end = 3/gb = AW122780/gi = 6098310/ug = Mm.21387/len = 148/STRA = for | | | | | | | |
| 7 | 93975_at | Cluster Incl AI853531 UI-M-BH0-ajd-f-01-0-UI s1 Mus musculus cDNA, 3 end/clone = UI-M-BH0-ajd-f-01-0-UI/ | P | 313 | P | 138.6 | P | 355.9 | P |
| | | clone_end = 3/gb = AI853531/gi = 5497437/ug = Mm 21679/len = 425/STRA = for | | | | | | | |
| 7 | 94060_at | Cluster Incl AI852623 UI-M-BH0-aiu-e-12-0-UI.s1 Mus musculus cDNA, 3 end/clone = UI-M-BH0-alu-e-12-0-UI/ | P | 446.6 | P | 425.3 | P | 449.1 | P |
| | | clone_end = 3/gb = AI852623/gi = 5496529/ug = Mm.22044/len = 416/STRA = for | | | | | | | |
| 7 | 94218_at | Cluster Incl AW121091.UI-M-BH2 3-aof-h-07-0-UI s1 Mus musculus cDNA, 3 end/clone = UI-M-BH2 3-aof-h-07-0-UI/ | P | 1382.5 | P | 988.5 | P | 1028.8 | P |
| | | clone_end = 3/gb = AW121091/gi = 6096424/ug = Mm.22227/len = 214/STRA = for | | | | | | | |
| 7 | 94238_at | Cluster Incl AW228316 up21f09 y1 Mus musculus cDNA, 5 end/clone = IMAGE-2655017/clone_end = 5/gb = AW228316/ | P | 219.3 | P | 155.6 | P | 260.6 | P |
| | | gi = 6557612/ug = Mm 22345/len = 193/STRA = for | | | | | | | |
| 7 | 94246_at | Cluster Incl J04103: E26 avian leukemia oncogene 2,3 domain/cds = (137,1543)/gb = J04103/gi = 193193/ug = Mm.22365/ | P | 674.6 | P | 672.2 | P | 405 | P |
| | | len = 1552/STRA = for | | | | | | | |
| 7 | 94256_at | Cluster Incl AI849533 UI-M-AH1-agt-h-01-0-UI.s1 Mus musculus cDNA, 3 end/clone = UI-M-AH1-agt-h-01-0-UI/ | P | 1019.8 | P | 908.6 | P | 953.7 | P |
| | | clone_end = 3/gb = AI849533/gi = 5493439/ug = Mm 22409/len = 360/STRA = for | | | | | | | |
| 7 | 94258_at | Cluster Incl L07918 Mus musculus GDP-dissociation inhibitor mRNA, preferentially expressed in hematopoietic cells, | P | 1134.3 | P | 868.9 | P | 914.6 | P |
| | | complete cds/cds = (32,634)/gb = L07918/gi = 193461/ug = Mm 2241/len = 1099/STRA = rev | | | | | | | |
| 7 | 94301_at | Cluster Incl AI843269.UI-M-AO1-aei-c-08-0-UI s1 Mus musculus cDNA, 3 end/clone = UI-M-AO1-aei-c-08-0-UI/ | P | 714.4 | P | 504.3 | P | 609.5 | P |
| | | clone_end = 3/gb = AI843269/gi = 5477482/ug = Mm 22602/len = 310/STRA = for | | | | | | | |
| 7 | 94303_at | Cluster Incl U11274 Mus musculus clone pmuAUF1-3 RNA-binding protein AUF1 mRNA, complete cds/cds = (142,951)/ | P | 1003.1 | P | 687.9 | P | 762.4 | P |
| | | gb = U11274/gi = 508269/ug = Mm 22617/len = 1322/STRA = for | | | | | | | |
| 7 | 95405_at | Cluster Incl AW045534: UI-M-BH1-aks-c-09-0-UI s1 Mus musculus cDNA, 3 end/clone = UI-M-BH1-aks-c-09-0-UI/ | P | 328.3 | P | 214.5 | P | 244.4 | P |
| | | clone_end = 3/gb = AW045534/gi = 5906063/ug = Mm 24209/len = 435/STRA = for | | | | | | | |
| 7 | 95413_at | Cluster Incl AI851332 UI-M-BH0-akh-b-07-0-UI s1 Mus musculus cDNA, 3 end/clone = UI-M-BH0-akh-b-07-0-UI/ | P | 854.4 | P | 924.1 | P | 1041.2 | P |
| | | clone_end = 3/gb = AI851332/gi = 5495238/ug = Mm 24272/len = 387/STRA = for | | | | | | | |
| 7 | 95477_at | Cluster Incl AW125185 UI-M-BH2 1-apz-g-10-0-UI s1 Mus musculus cDNA, 3 end/clone = UI-M-BH2.1-apz-g-10-0-UI/ | P | 1552.3 | P | 1267.1 | P | 1434.1 | P |
| | | clone_end = 3/gb = AW125185/gi = 6100715/ug = Mm.24847/len = 215/STRA = for | | | | | | | |
| 7 | 96047_at | Cluster Incl U63146 Mus musculus retinol binding protein (RBP) mRNA, complete cds/cds = (0,605)/gb = U63146/ | P | 211.8 | P | 216.8 | P | 164.8 | P |
| | | gi = 1515449/ug = Mm 2605/len = 606/STRA = for | | | | | | | |
| 7 | 96083_s_at | Cluster Incl AI226368 uf07b12.y1 Mus musculus cDNA, 5 end/clone = IMAGE-1510655/clone_end = 5/gb = AI226368/ | P | 1619.8 | P | 492.5 | P | 734.6 | P |
| | | gi = 3809421/ug = Mm 26617/len = 340/STRA = for | | | | | | | |
| 7 | 96084_at | Cluster Incl AB017020 Mus musculus mRNA for JKTBP, complete cds/cds = (123,1028)/gb = AB017020/gi = 4514630/ | P | 268.2 | P | 245.6 | P | 185.9 | P |
| | | ug = Mm 26617/len = 1307/STRA = for | | | | | | | |
| 7 | 96633_s_at | Cluster Incl AA529583 vi41d07 r1 Mus musculus cDNA, 5 end/clone = IMAGE-906349/clone_end = 5/gb = AA529583/ | P | 1596.8 | P | 1151.6 | P | 969.9 | P |
| | | gi = 2272289/ug = Mm 27218/len = 546/STRA = for | | | | | | | |
| 7 | 96664_at | Cluster Incl AB025966 Mus musculus mRNA for KIAA312p, partial cds/cds = (0,1123)/gb = AB025966/gi = 5931572/ | P | 904.6 | P | 571.4 | P | 772.5 | P |
| | | ug = Mm 27372/len = 1638/STRA = for | | | | | | | |
| 7 | 97211_at | Cluster Incl AI747444: ul15f07.x1 Mus musculus cDNA, 3 end/clone = IMAGE-2076901/clone_end = 3/gb = AI747444/ | P | 369.8 | P | 340.8 | P | 506.3 | P |
| | | gi = 5125708/ug = Mm 27714/len = 657/STRA = for | | | | | | | |
| 7 | 97240_g_at | Cluster Incl AI845106.UI-M-BG0-ahs-a-04-0-UI s1 Mus musculus cDNA, 3 end/clone = UI-M-BG0-ahs-a-04-0-UI/ | P | 1132.4 | P | 1092.7 | P | 1220.5 | P |
| | | clone_end = 3/gb = AI845106/gi = 5489012/ug = Mm 27839/len = 141/STRA = for | | | | | | | |
| 7 | 97819_at | Cluster Incl AI843119 UI-M-AK1-aes-f-05-0-UI s1 Mus musculus cDNA, 3 end/clone = UI-M-AK1-aes-f-05-0-UI/ | P | 774.2 | P | 486.2 | P | 590.7 | P |
| | | clone_end = 3/gb = AI843119/gi = 5477332/ug = Mm 282/len = 199/STRA = for | | | | | | | |
| 7 | 97864_at | Cluster Incl AW258842 um74d01 y1 Mus musculus cDNA, 5 end/clone = IMAGE-2300929/clone_end = 5/gb = AW258842/ | P | 256.5 | P | 185.8 | P | 267.8 | P |
| | | gi = 6631823/ug = Mm 28327/len = 321/STRA = for | | | | | | | |
| 7 | 97865_g_at | Cluster Incl AW258842 um74d01 y1 Mus musculus cDNA, 5 end/clone = IMAGE-2300929/clone_end = 5/gb = AW258842/ | P | 719.1 | P | 563.9 | P | 778.9 | P |
| | | gi = 6631823/ug = Mm 28327/len = 321/STRA = for | | | | | | | |
| 7 | 98937_at | Cluster Incl AW049795 UI-M-BH1-anm-1-07-0-UI s1 Mus musculus cDNA, 3 end/clone = UI-M-BH1-anm-f-07-0-UI/ | P | 917.7 | P | 999.6 | P | 1034.7 | P |
| | | clone_end = 3/gb = AW049795/gi = 5910324/ug = Mm 28689/len = 562/STRA = for | | | | | | | |
| 7 | 94448_at | Cluster Incl AJ006289: Mus musculus mRNA for bcl-10 protein/cds = (78,779)/gb = AJ006289/gi = 4049471/ug = Mm.28782/ | P | 585.1 | P | 335.4 | P | 296.2 | P |
| | | len = 1756/STRA = for | | | | | | | |
| 7 | 94516_f_at | Cluster Incl M55181: Preproenkephalin 2/cds = UNKNOWN/gb = M55181/gi = 201032/ug = Mm.2899/len = 1408/STRA = for | P | 2705.8 | P | 1156.4 | P | 1970.9 | P |
| 7 | 95709_at | Cluster Incl AW012491 uo05f02 y1 Mus musculus cDNA, 5 end/clone = IMAGE-2582523/clone_end = 5/gb = AW012491/ | P | 1115.9 | P | 1107.3 | P | 1141.5 | P |
| | | gi = 5861269/ug = Mm 29703/len = 528/STRA = for | | | | | | | |
| 7 | 96254_at | Cluster Incl AB028272 Mus musculus hsp40 mRNA for heat shock protein 40, complete cds/cds = (139,1161)/ | P | 316.3 | P | 358 | P | 297.9 | P |
| | | gb = AB028272/gi = 6531981/ug = Mm 2982/len = 2279/STRA = for | | | | | | | |
| 7 | 96271_at | Cluster Incl AA914105 vy92c01.r1 Mus musculus cDNA, 5 end/clone = IMAGE-1313664/clone_end = 5/gb = AA914105/ | P | 1692.5 | P | 1137.4 | P | 1256.1 | P |
| | | gi = 3053497/ug = Mm 29853/len = 415/STRA = for | | | | | | | |
| 7 | 96288_at | Cluster Incl AI648831 uk29f08.x1 Mus musculus cDNA, 3 end/clone = IMAGE-1970439/clone_end = 3/gb = AI648831/ | P | 380.4 | P | 303 | P | 428.3 | P |
| | | gi = 4729665/ug = Mm 29889/len = 526/STRA = for | | | | | | | |
| 7 | 96325_at | Cluster Incl AW124874 UI-M-BH2.1-apu-b-08-0-UI s1 Mus musculus cDNA, 3 end/clone = UI-M-BH2 1-apu-b-08-0-UI/ | P | 288.6 | P | 240.9 | P | 312.7 | P |
| | | clone_end = 3/gb = AW124874/gi = 6100404/ug = Mm 29949/len = 429/STRA = for | | | | | | | |
| 7 | 96336_at | Cluster Incl AI844626 UI-M-AL1-ahr-a-04-0-UI.s1 Mus musculus cDNA, 3 end/clone = UI-M-AL1-ahr-a-04-0-UI/ | P | 495.2 | P | 339.3 | P | 545.1 | P |
| | | clone_end = 3/gb = AI844626/gi = 5488532/ug = Mm.29975/len = 568/STRA = for | | | | | | | |
| 7 | 96841_at | Cluster Incl AW046627 UI-M-BH1-ald-c-09-0-UI s1 Mus musculus cDNA, 3 end/clone = UI-M-BH1-ald-c-09-0-UI/ | P | 454.7 | P | 363.5 | P | 305.8 | P |
| | | clone_end = 3/gb = AW046627/gi = 5907158/ug = Mm 30018/len = 268/STRA = for | | | | | | | |
| 7 | 96920_at | Cluster Incl AW125478: UI-M-BH2.2-aqm-c-03-0-UI s1 Mus musculus cDNA, 3 end/clone = UI-M-BH2 2-aqm-c-03-0-UI/ | P | 986.1 | P | 918.9 | P | 1076.6 | P |
| | | clone_end = 3/gb = AW125478/gi = 6101008/ug = Mm 30156/len = 468/STRA = for | | | | | | | |
| 7 | 97450_s_at | Cluster Incl AA986258 uc74b03.y1 Mus musculus cDNA, 5 end/clone = IMAGE-1431341/clone_end = 5/gb = AA986258/ | P | 363.9 | P | 322.4 | P | 256.4 | P |
| | | gi = 3167582/ug = Mm 30250/len = 552/STRA = for | | | | | | | |
| 7 | 97462_at | Cluster Incl AI847584 UI-M-AP1-agg-a-06-0-UI s1 Mus musculus cDNA, 3 end/clone = UI-M-AP1-agg-a-06-0-UI/ | P | 745.4 | P | 623.6 | P | 717.7 | P |
| | | clone_end = 3/gb = AI847584/gi = 5491490/ug = Mm 3035/len = 340/STRA = for | | | | | | | |
| 7 | 97492_at | Cluster Incl AW121746: UI-M-BH2.3-aoe-c-09-0-UI s1 Mus musculus cDNA, 3 end/clone = UI-M-BH2 3-aoe-c-09-0-UI/ | P | 311.5 | P | 245.9 | P | 248.1 | P |
| | | clone_end = 3/gb = AW121746/gi = 6097081/ug = Mm 31076/len = 293/STRA = for | | | | | | | |
| 7 | 97496_f_at | Cluster Incl AW048944.UI-M-BH1-amo-e-07-0-UI.s1 Mus musculus cDNA, 3 end/clone = UI-M-BHI-amo-e-07-0-UI/ | P | 984.1 | P | 1098.1 | P | 882 | P |
| | | clone_end = 3/gb = AW048944/gi = 5909473/ug = Mm 3124/len = 296/STRA = for | | | | | | | |
| 7 | 97538_at | Cluster Incl M19279 Beta-glucuronidase structural/cds = (12,1958)/gb = M19279/gi = 193524/ug = Mm 3317/len = 2454/ | P | 194.5 | P | 141.5 | P | 76.4 | P |
| | | STRA = for | | | | | | | |
| 7 | 98084_at | Cluster Incl AI849834: UI-M-AL1-aho-h-09-0-UI s1 Mus musculus cDNA, 3 end/clone = UI-M-AL1-aho-h-09-0-UI/ | P | 1178.3 | P | 1240.5 | P | 1297.6 | P |
| | | clone_end = 3/gb = AI849834/gi = 5493780/ug = Mm 34577/len = 464/STRA = for | | | | | | | |
| 7 | 98110_at | Cluster Incl AI853375: UI-M-BH0-air-e-09-0-UI s1 Mus musculus cDNA, 3 end/clone = UI-M-BH0-air-e-09-0-UI/ | P | 414.8 | P | 322.3 | P | 398.6 | P |
| | | clone_end = 3/gb = AI853375/gi = 5497281/ug = Mm 34810/len = 388/STRA = for | | | | | | | |
| 7 | 98593_at | Cluster Incl AJ006215: Mus musculus mRNA for CMP-N-acetylneuraminic acid synthetase/cds = (0,1298)/gb = AJ006215/ | P | 269.2 | P | 193.4 | P | 323.3 | P |
| | | gi = 3413319/ug = Mm.3820/len = 1687/STRA = for | | | | | | | |
| 7 | 99082_at | Cluster Incl L07063 Mus musculus FKBP65 binding protein mRNA, complete cds/cds = (93,1838)/gb = L07063/gi = 894161/ | P | 385.3 | P | 387.4 | P | 402.8 | P |
| | | ug = Mm 3894/len = 2597/STRA = for | | | | | | | |
| 7 | 99127_at | Cluster Incl X61506 Mouse E46 mRNA for E46 protein/cds = (198,1478)/gb = X61506/gi = 50794/ug = Mm 4098/len = 1668/ | P | 1183 | P | 10750.3 | P | 1252.8 | P |
| | | STRA = for | | | | | | | |
| 7 | 99133_at | Cluster Incl X14309 Antigen identified by monoclonal antibodies 4F2/cds = (92,1672)/gb = X14309/gi = 49749/ug = Mm 4114/ | P | 1300 | P | 1191.7 | P | 1086.4 | P |
| | | len = 1828/STRA = for | | | | | | | |
| 7 | 99563_at | Cluster Incl AW124681 UI-M-BH2.1-apw-g-06-0-UI s1 Mus musculus cDNA, 3 end/clone = UI-M-BH2 1-apw-g-06-0-UI/ | P | 507.9 | P | 438.2 | P | 527.7 | P |
| | | clone_end = 3/gb = AW124681/gi = 6100211/ug = Mm 42193/len = 421/STRA = for | | | | | | | |
| 7 | 99602_at | Cluster Incl AF064088 Mus musculus transcription factor GIF mRNA, complete cds/cds = (109,1548)/gb = AF064088/ | P | 585.3 | P | 336.3 | P | 411.6 | P |
| | | gi = 3139151/ug = Mm 4292/len = 3036/STRA = for | | | | | | | |
| 7 | 100094_at | Cluster Incl U88539 Mus musculus chromatin structural protein homolog Supt5hp (Supt5h) mRNA, complete cds/ | P | 516 | P | 412 .7 | P | 493.7 | P |
| | | cds = (61,3309)/gb = U88539/gi = 2754751/ug = Mm 460/len = 3538/STRA = for | | | | | | | |
| 7 | 100115_at | Cluster Incl AW124128 UI-M-BH2.1-apr-d-07-0-UI s1 Mus musculus cDNA, 3 end/clone = UI-M-BH2 1-apr-d-07-0-UI/ | P | 346 | P | 274.4 | P | 397.8 | P |
| | | clone_end = 3/gb = AW124128/gi = 6099658/ug = Mm 46624/len = 357/STRA = for | | | | | | | |
| 7 | 100126_at | Cluster Incl AA967263 vz70e04 r1 Mus musculus cDNA, 5 end/clone = IMAGE-1331838/clone_end = 5/gb = AA967263/ | P | 850.3 | P | 388.7 | P | 601.3 | P |
| | | gi = 3141156/ug = Mm 4748/len = 432/STRA = for | | | | | | | |
| 7 | 100554_at | Cluster Incl AF053367 Mus musculus carboxyl terminal LIM domain protein (Ldb3) mRNA, complete cds/cds = (60,1040)/ | P | 417.9 | P | 361.9 | P | 599.7 | P |
| | | gb = AF053367/gi = 2996195/ug = Mm 5567/len = 1256/STRA = for | | | | | | | |
| 7 | 100555_at | Cluster Incl AI846152.UI-M-AN1-afj-h-05-0-UI.s2 Mus musculus cDNA, 3 end/clone = UI-M-AN1-afj-h-05-0-UI/ | P | 681.1 | P | 699.4 | P | 762.4 | P |
| | | clone_end = 3/gb = AI846152/gi = 5490058/ug = Mm 56/len = 408/STRA = for | | | | | | | |
| 7 | 100557_g_at | Cluster Incl AW121930-UI-M-BH2.3-aoj-d-12-0-UI s1 Mus musculus cDNA, 3 end/clone = UI-M-BH2 3-aoj-d-12-0-UI/ | P | 867.2 | P | 1078.6 | P | 1099.5 | P |
| | | clone_end = 3/gb = AW121930/gi = 6097374/ug = Mm 5612/len = 467/STRA = for | | | | | | | |
| 7 | 100579_s_at | Cluster Incl U91848 Mus musculus clathrin light chain 2 mRNA, complete cds/cds = (108,815)/gb = U91848/gi = 1916987/ | P | 1086.5 | P | 782.6 | P | 1029.4 | P |
| | | ug = Mm 6072/len = 1089/STRA = for | | | | | | | |
| 7 | 100613_at | Cluster Incl AF030635: Mus musculus FK-506 binding protein homolog (SAM11) mRNA, complete cds/cds = (28,1095)/ | P | 878.2 | P | 851 | P | 791 | P |
| | | gb = AF030635/gi = 2623223/ug = Mm 6565/len = 1514/STRA = for | | | | | | | |
| 7 | 100622_at | Cluster Incl AF093857: untitled/cds = (49,723)/gb = AF093857/gi = 4139185/ug = Mm 6587/len = 1436/STRA = for | P | 1058.2 | P | 570.8 | P | 675.3 | P |
| 7 | 101078_at | Cluster Incl Y16258 Basigin/cds = (0,773)/gb = Y18258/gi = 2808471/ug = Mm 726/len = 1244/STRA = for | P | 1925.7 | P | 2318.7 | P | 2110.6 | P |
| 7 | 101086_f_at | Cluster Incl X63866 Mus musculus mRNA for cellular nucleic acid binding protein/cds = (104,616)/gb = X63866/gi = 50470/ | P | 2173.3 | P | 1418.6 | P | 1483.2 | P |
| | | ug = Mm 7335/len = 1492/STRA = for | | | | | | | |
| 7 | 101088_f_at | Cluster Incl U20326 Mus musculus cellular nucleic acid binding protein mRNA, complete cds/cds = (119,655)/gb = U20326/ | P | 1917.4 | P | 1633.4 | P | 1691 | P |
| | | gi = 854674/ug = Mm 7335/len = 1519/STRA = for | | | | | | | |
| 7 | 101095_at | Cluster Inc L23769 Microfibril-associated glycoprotein/cds = (80,631)/gb = L23769/gi = 440831/ug = Mm 7386/len = 1020/ | P | 1046.6 | P | 750.6 | P | 1321.6 | P |
| | | STRA = for | | | | | | | |
| 7 | 101107_at | Cluster Incl U81829 Calumenin/cds = (35,982)/gb = U81829/gi = 2323276/ug = Mm 7515/len = 3147/STRA = for | P | 1555.3 | H | 974.8 | P | 937.7 | P |
| 7 | 101110_at | Cluster Incl AF064749 Mus musculus type VI collagen alpha 3 subunit mRNA, complele cds/cds = (61,8034)/gb = AF064749/ | P | 3456.8 | P | 2752.1 | P | 3660.1 | P |
| | | gi = 3236369/ug = Mm 7562/len = 8775/STRA = for | | | | | | | |
| 7 | 101499_at | Cluster Incl U94479 Mus musculus Integrin binding protein kinase mRNA, complete cds/cds = (113,1471)/gb = U94479/ | P | 852.4 | P | 830.8 | P | 1069.9 | P |
| | | gi = 2739449/ug = Mm 8131/len = 1750/STRA = for | | | | | | | |
| 7 | 101514_at | Cluster Incl AF041433 Mus musculus bet3 (Bet3) mRNA, complete cds/cds = (82,624)/gb = AF041433/gi = 2791805/ | P | 330.2 | P | 412.2 | P | 372.9 | P |
| | | ug = Mm 8392/len = 753/STRA = for | | | | | | | |
| 7 | 101991_at | Cluster Incl D16215 Mouse mRNA for flavin-containing monooxygenase, complete cds/cds = (206,1804)/gb = D16215/ | P | 288 | P | 184.3 | P | 258.7 | P |
| | | gi = 286077/ug = Mm 976/len = 2126/STRA = rev | | | | | | | |
| 7 | 102044_at | Cluster Incl AF100777 Mus musculus connective tissue growth factor related protein WISP-1 (Wisp1) mRNA, complete cds/ | P | 233.6 | P | 130.9 | P | 218.5 | P |
| | | cds = (78,1181)/gb = AF100777/gi = 4028576/ug = Mm 10222/len = 1766/STRA = for | | | | | | | |
| 7 | 102322_at | Cluster Incl AF061017 UDP-glucose dehydrogenase/cds = (110,1591)/gb = AF061017/gi = 3127128/ug = Mm 10709/ | P | 1425.6 | P | 995.8 | P | 1480.9 | P |
| | | len = 2328/STRA = for | | | | | | | |
| 7 | 102395_at | Cluster Incl Z38110 M musculus PMP22 mRNA for peripheral myelin protein/cds = (0,482)/gb = Z38110/gi = 558353/ | P | 949.2 | P | 845.4 | P | 1145.6 | P |
| | | ug = Mm 1237/len = 483/STRA = for | | | | | | | |
| 7 | 102752_at | Cluster Incl AF072697 Mus musculus SHYC (Shyc) mRNA, complete cds/cds = (33,3794)/gb = AF072697/gi = 3293550/ | P | 1028.5 | P | 985.7 | P | 998.6 | P |
| | | ug = Mm.12912/len = 4112/STRA = for | | | | | | | |
| 7 | 102772_at | Cluster Incl J02995 Abelson murine leukemia oncogene/cds = (3565,4056)/gb = J02995/gi = 191566/ug = Mm 1318/ | P | 261.7 | P | 184 | P | 265.2 | P |
| | | len = 5058/STRA = for | | | | | | | |
| 7 | 102811_at | Cluster Incl X96639 Exostoses (multiple) 1/cds = (594,2831)/gb = X96639/gi = 1235558/ug = Mm 1424/len = 2965/STRA = for | P | 470.5 | P | 577.8 | P | 494.7 | P |
| 7 | 102983_at | Cluster Incl U58992.MAD homolog 1 (Drosophila)/cds = (70,1467)/gb = U58992/gi = 1518644/ug = Mm 15185/len = 1639/ | P | 426.3 | P | 333 | P | 333.9 | P |
| | | STRA = for | | | | | | | |
| 7 | 102984_g_at | Cluster Incl U58992: MAD homolog 1 (Drosophila)/cds = (70,1467)/gb = U58992/gi = 1518644/ug = Mm 15185/len = 1639/ | P | 893.4 | P | 853.9 | P | 849.3 | P |
| | | STRA = for | | | | | | | |
| 7 | 103054_at | Cluster Incl U37500: Mus musculus RNA polymerase II largest subunit (RPB1) mRNA, complete cds/cds = (69,5969)/ | P | 491.3 | P | 450.1 | P | 488.4 | P |
| | | gb = U37500/gi = 2145090/ug = Mm.16533/len = 8270/STRA = rev | | | | | | | |
| 7 | 103100_at | Cluster Incl AI788543 uk46c09 x1 Mus musculus cDNA, 3 end/clone = IMAGE-1972048/clone_end = 3/gb = AI788543/ | P | 647.8 | P | 509.5 | P | 475.1 | P |
| | | gi-5336259/ug = Mm 17569/len = 656/STRA = for | | | | | | | |
| 7 | 103353_f_at | Cluster Incl D50834 Cytochrome P450. subfamily IV B, polypeptide 1/cds = (18,1553)/gb = D50834/gi = 854665/ | P | 543.7 | P | 457.8 | P | 480.6 | P |
| | | ug = Mm 1840/len = 1871/STRA = for | | | | | | | |
| 7 | 103379_at | Cluster Incl AW046579 UI-M-BH1-alb-e-03-0-UI s1 Mus musculus cDNA, 3 end/clone = UI-M-BH1-alb-e-03-0-UI/ | P | 177.8 | P | 36.6 | P | 147.2 | P |
| | | clone_end = 3/gb = AW046579/gi = 5907108/ug = Mm 18616/len = 301/STRA = rev | | | | | | | |
| 7 | 103608_at | Cluster Incl AI842638.UI-M-AQ1-aed-f-04-0-UI s1 Mus musculus cDNA, 3 end/clone = UI-M-AQ1-aed-f-04-0-UI/ | P | 272.5 | P | 296.8 | P | 290.8 | P |
| | | clone_end = 3/gb = AI842636/gi = 5476849/ug = Mm 20175/len = 311/STRA = for | | | | | | | |
| 7 | 104100_at | Cluster Incl AI845915: UI-M-AK1-aex-d-02-0-UI.s1 Mus musculus cDNA, 3 end/clone = UI-M-AK1-aex-d-02-0-UI/ | P | 2320.8 | P | 1653.7 | P | 2569.9 | P |
| | | clone_end = 3/gb = AI845915/gi = 5489821/ug = Mm.21864/len = 208/STRA = for | | | | | | | |
| 7 | 104343_f_at | Cluster Incl AI845798-UI-M-AQ1-aeb-h-07-0-UI s1 Mus musculus cDNA, 3 end/clone = UI-M-AQ1-aeb-h-07-0-UI/ | P | 291.5 | P | 175.9 | P | 214.4 | P |
| | | clone_end = 3/gb = AI845798/gi = 5489704/ug = Mm 22526/len = 184/STRA = for | | | | | | | |
| 7 | 104587_at | Cluster Incl U69176: Laminin, alpha 4/cds = (386,5833)/gb = U69176/gi = 1568624/ug = Mm 2399/len = 5993/STRA = for | P | 514.1 | P | 368 | P | 389.7 | P |
| 7 | 104712_at | Cluster Incl L00039 Myelocytomalosis oncogene/cds = (15,1334)/gb = L00039/gi = 192621/ug = Mm 2444/len = 1707/ | P | 110.4 | P | 123.3 | P | 90.8 | P |
| | | STRA = for | | | | | | | |
| 7 | 104756_at | Cluster Incl AA739415: vv53c01 r1 Mus musculus cDNA, 5 end/clone = IMAGE-1226112/clone_end = 5/gb = AA739415/ | P | 381.5 | P | 423.8 | P | 469.4 | P |
| | | gi = 2775601/ug = Mm.24788/len = 451/STRA = for | | | | | | | |
| 7 | 94394_at | Cluster Incl M21019: Harvey rat sarcoma oncogene, subgroup R/cds = (11,667)/gb = M21019/gi = 200676/ug = Mm 257/ | P | 171 | P | 104.7 | P | 291.1 | P |
| | | len = 949/STRA = rev | | | | | | | |
| 7 | 94415_at | Cluster Incl AA710439 vt42e01 r1 Mus musculus cDNA, 5 end/clone = IMAGE-1165752/clone_end = 5/gb = AA710439/ | P | 322.6 | P | 294.6 | P | 273.8 | P |
| | | gi = 2720357/ug = Mm 26147/len = 510/STRA = for | | | | | | | |
| 7 | 94942_at | Cluster Incl AI854108: UI-M-BH0-ajw-c-12-0-UI s1 Mus musculus cDNA, 3 end/clone = UI-M-BH0-ajw-c-12-0-UI/ | P | 287.4 | P | −4.1 | P | 41 | P |
| | | clone_end = 3/gb = AI854108/gi = 5498014/ug = Mm 26944/len = 581/STRA = rev | | | | | | | |
| 7 | 95609_at | Cluster Incl AA869927: vq19d11.r1 Mus musculus cDNA, 5 end/clone = IMAGE-1094709/clone_end = 5/gb = AA869927/ | P | 352.5 | P | 309.8 | P | 368.8 | P |
| | | gi = 2965372/ug = Mm 27987/len = 497/STRA = for | | | | | | | |
| 7 | 96178_at | Cluster Incl AI850636 UI-M-BG1-aik-e-04-0-UI.s1 Mus musculus cDNA, 3 end/clone = UI-M-BG1-aik-e-04-0-UI/ | P | 353.5 | P | 292.9 | P | 351 | P |
| | | clone_end = 3/gb = AI850636/gi = 5494542/ug = Mm 28426/len = 391/STRA = for | | | | | | | |
| 7 | 96733_at | Cluster Incl AW124597 UI-M-BH2 1-apt-h-01-0-UI s1 Mus musculus cDNA, 3 end/clone = UI-M-BH2 1-apt-h-01-0-UI/ | P | 506.3 | P | 565 | P | 461.4 | P |
| | | clone_end = 3/gb = AW124597/gi = 6100132/ug = Mm 28893/len = 499/STRA = for | | | | | | | |
| 7 | 96767_at | Cluster Incl AF098633 Mus musculus GLUT4 vesicle protein mRNA, partial cds/cds = (0,791)/gb = AF098633/gi = 4200441/ | P | 356.6 | P | 200.8 | P | 155.8 | P |
| | | ug = Mm 29010/len = 1192/STRA = rev | | | | | | | |
| 7 | 97359_at | Cluster Incl AI851160 UI-M-BH0-akg-f-01-0-UI.s1 Mus musculus cDNA, 3 end/clone = UI-M-BH0-akg-f-01-0-UI/ | P | 458.3 | P | 314.1 | P | 390.9 | P |
| | | clone_end = 3/gb = AI851160/gi = 5495031/ug = Mm 29649/len = 447/STRA = for | | | | | | | |
| 7 | 98474_r_at | Cluster Incl U83903: Tumor necrosis factor induced protein 6/cds = (63,890)/gb = U83903/gi = 2062474/ug = Mm 3509/ | P | 177 | P | 60.5 | P | 92.3 | P |
| | | len = 1605/STRA = for | | | | | | | |
| 7 | 98475_at | Cluster Incl U69262: Mus musculus matrilin-2 precursor mRNA, complete cds/cds = (250,3120)/gb = U69262/gi = 2072791/ | P | 258 | P | 129.5 | P | 262 | P |
| | | ug = Mm 3511/len = 3571/STRA = for | | | | | | | |
| 7 | 98508_s_at | Cluster Incl D84376: Mus musculus mRNA for Phosphatidic acid phosphatase, complete cds/cds = (286,1137)/gb = D84376/ | P | 285 | P | 145.6 | P | 335.5 | P |
| | | gi = 1487872/ug = Mm 35836/len = 1212/STRA = for | | | | | | | |
| 7 | 99031_at | Cluster Incl AI854005 UI-M-BH0-arv-h-09-0-UI.s1 Mus musculus cDNA, 3 end/clone = UI-M-BH0-arv-h-09-0-UI/ | P | 345.9 | P | 246.5 | P | 279.2 | P |
| | | clone_end = 3/gb = AI854005/gi = 5497911/ug = Mm.38930/len = 538/STRA = for | | | | | | | |
| 7 | 99038_at | Cluster Incl L24554 Mus musculus adenylosuccinate synthetase mRNA, complete cds/cds = (82,1452)/gb = L24554/ | P | 442.9 | P | 249.5 | P | 515.8 | P |
| | | gi = 404058/ug = Mm 39061/len = 1559/STRA = for | | | | | | | |
| 7 | 99039_g_at | Cluster Incl L24554 Mus musculus adenylosuccinate synthetase mRNA, complete cds/cds = (82,1452)/gb = L24554/ | P | 418.9 | P | 325.9 | P | 388.1 | P |
| | | gi = 404056/ug = Mm 39061/len = 1559/STRA = for | | | | | | | |
| 7 | 99535_at | Cluster Incl AW047630 UI-M-BH1-akm-g-06-0-UI s1 Mus musculus cDNA, 3 end/clone = UI-M-BH1-akm-g-06-0-UI/ | P | 458.8 | P | 406.6 | P | 295.4 | P |
| | | clone_end = 3/gb = AW047630/gi = 5908159/ug = Mm 42187/len = 385/STRA = for | | | | | | | |
| 7 | 100442_at | Cluster Incl AA656541 vs22a03 r1 Mus musculus cDNA, 5 end/clone = IMAGE-1138924/clone_end = 5/gb = AA656541/ | P | 221.3 | P | 179.4 | P | 207.4 | P |
| | | gi = 2592695/ug = Mm 4810/len = 305/STRA = for | | | | | | | |
| 7 | 100466_f_at | Cluster Incl AA655823: vs48b07 r1 Mus musculus cDNA, 5 end/clone = IMAGE-1149493/clone_end = 5/gb = AA655823/ | P | 1273 | P | 862.5 | P | 1023 | P |
| | | gi = 2591977/ug = Mm.4923/len = 356/STRA = for | | | | | | | |
| 7 | 100974_at | Cluster Incl AI844250 UI-M-AL1-ahk-g-10-0-UI s1 Mus musculus cDNA, 3 end/clone = UI-M-AL1-ahk-g-10-0-UI/ | P | 277.1 | P | 283.7 | P | 241.1 | P |
| | | clone_end = 3/gb = AI844250/gi = 5488156/ug = Mm.6667/len = 237/STRA = rev | | | | | | | |
| 7 | 101921_at | Cluster Incl D86563: RAB4A, member RAS oncogene family/cds = (129,770)/gb = D86583/gi = 2706877/ug = Mm 9221/ | P | 196.3 | P | 160.8 | P | 208.8 | P |
| | | len = 1385/STRA = for | | | | | | | |
| 7 | 103310_at | Cluster Incl AA220427 mv70b05 r1 Mus musculus cDNA, 5 end/clone = IMAGE-660369/clone_end = 5/gb = AA220427/ | P | 129.3 | P | 143.3 | P | 113.8 | P |
| | | gi = 1838880/ug = Mm 21184/len = 261/STRA = for | | | | | | | |
| 7 | 103549_at | Cluster Incl AW061260: UI-M-BH1-amj-f-05-0-UI s2 Mus musculus cDNA, 3 end/clone = UI-M-BH1-amj-f-05-0-UI/ | P | 588 | P | 497.7 | P | 616.8 | P |
| | | clone_end = 3/gb = AW061260/gi = 6009011/ug = Mm 23742/len = 185/STRA = for | | | | | | | |
| 7 | 103555_at | Cluster Incl AI843520 UI-M-AO1-aeh-c-10-0-UI s1 Mus musculus cDNA, 3 end/clone = UI-M-AO1-aeh-c-10-0-UI/ | P | 2567.7 | P | 2087.9 | P | 2164.8 | P |
| | | clone_end = 3/gb = AI843520/gi = 5477733/ug = Mm 23842/len = 387/STRA = for | | | | | | | |
| 7 | 104235_at | Cluster Incl AW122882 UI-M-BH2.1-apa-c-06-0-UI s1 Mus musculus cDNA, 3 end/clone = UI-M-BH2 1-apa-c-06-0-UI/ | P | 1256.2 | P | 1027 | P | 866.8 | P |
| | | clone_end = 3/gb = AW122882/gi = 6098412/ug = Mm 28117/len = 316/STRA = for | | | | | | | |
| 7 | 104410_at | Cluster Incl AW124785: UI-M-BH2.1-aqa-h-06-0-UI s1 Mus musculus cDNA, 3 end/clone = UI-M-BH2 1-aqa-h-06-0-UI/ | P | 848.7 | P | 881.8 | P | 920.7 | P |
| | | clone_end = 3/gb = AW124785/gi = 6100315/ug = Mm 2883/len = 417/STRA = for | | | | | | | |
| 7 | 104464_a_at | Cluster Incl AI642389: vs90e02 x1 Mus musculus cDNA, 3 end/clone = IMAGE-1153562/clone_end = 3/gb = AI642389/ | P | 471.8 | P | 360.6 | P | 356.1 | P |
| | | gi = 4720864/ug = Mm 29644/len = 309/STRA = for | | | | | | | |
| 7 | 104494_at | Cluster Incl AI642098: ub75g10.x1 Mus musculus cDNA, 3 end/clone = IMAGE-1383618/clone_end = 3/gb = AI642098/ | P | 580.1 | P | 484.4 | P | 526 | P |
| | | gi = 4720573/ug = Mm 3038/len = 448/STRA = for | | | | | | | |
| 7 | 92925_at | Cluster Incl M61007 CCAAT/enhancer binding protein (C/EBP), beta/cds = (16,906)/gb = M61007/gi = 191775/ug = Mm 4883/ | P | 1969.6 | P | 1882.6 | P | 1573.3 | P |
| | | len = 1416/STRA = rev | | | | | | | |
| 7 | 93440_at | Cluster Incl AA692530 vt59h11.r1 Mus musculus cDNA, 5 end/clone = IMAGE-1167429/clone_end = 5/gb = AA692530/ | P | 371.4 | P | 279.7 | P | 292.9 | P |
| | | gi = 2693468/ug = Mm 6636/len = 414/STRA = for | | | | | | | |
| 7 | 93482_at | Cluster Incl AI117835 uc41b01 r1 Mus musculus cDNA, 5 end/clone = IMAGE-1400521/clone_end = 5/gb = AI117835/ | P | 676.8 | P | 461.3 | P | 581 | P |
| | | gi = 3518159/ug = Mm 7128/len = 397/STRA = rev | | | | | | | |
| 7 | 93924_f_at | Cluster Incl M13443 Mouse alpha-tubulin isotype M-alpha-7 mRNA, complete cds/cds = (39,1391)/gb = M13443/gi = 202218/ | P | 862.2 | P | 754.1 | P | 878.6 | P |
| | | ug = Mm 89866/len = 1424/STRA = for | | | | | | | |
| 7 | 97740_at | Cluster Incl AI642662 vw01d07.x1 Mus musculus cDNA, 3 end/clone = IMAGE-1230637/clone_end = 3/gb = AI642662/ | P | 122.1 | P | 152.5 | P | 205.9 | P |
| | | gi = 4721137/ug = Mm 3994/len = 307/STRA = for | | | | | | | |
| 7 | 100708_at | Cluster Incl X13605: Murine mRNA for replacement variant histone H3.3/cds = (73,483)/gb = X13605/gi = 51197/ | P | 4083.4 | P | 3748.4 | P | 4624.8 | P |
| | | ug = Mm.18516/len = 1619/STRA = for | | | | | | | |
| 7 | 93842_at | Cluster Incl AI196645: ui53d07 y1 Mus musculus cDNA, 5 end/clone = IMAGE-1886125/clone_end = 5/gb = AI196645/ | P | 287.6 | P | −16.4 | P | 7.6 | P |
| | | gi = 3749251/ug = Mm 21622/len = 1009/STRA = for | | | | | | | |
| 7 | 93974_at | Cluster Incl AW212475 uo89c05.x1 Mus musculus cDNA, 3 end/clone = IMAGE-2649704/clone_end = 3/gb = AW212475/ | P | 231.1 | P | 229.3 | P | 158.5 | A |
| | | gi = 6518562/ug = Mm 21679/len = 230/STRA = for | | | | | | | |
| 7 | 93996_at | Cluster Incl X01026: Mouse mRNA fragment for repetitive element B2/cds = (0,119)/gb = X01026/gi = 53957/ug = Mm 21758/ | P | 198.6 | P | 119.1 | P | 338.6 | P |
| | | len = 354/STRA = for | | | | | | | |
| 7 | 95469_at | Cluster Incl AA734444 vr90g04.r1 Mus musculus cDNA, 5 end/clone = IMAGE-1136022/clone_end = 5/gb = AA734444/ | P | 162.8 | P | 185.9 | P | 145.2 | A |
| | | gi = 2756111/ug = Mm 24786/len = 252/STRA = for | | | | | | | |
| 7 | 96637_at | Cluster Incl U33005 Mus musculus tbc1 mRNA, complete cds/cds = (402,3827)/gb = U33005/gi = 988220/ug = Mm 27271/ | P | 280.1 | P | 260.1 | P | 259.8 | A |
| | | len = 4039/STRA = for | | | | | | | |
| 7 | 96703_at | Cluster Incl AB029448: Mus musculus mRNA for Dixin-1, complete cds/cds = (212,2539)/gb = AB029448/gi = 6520236/ | P | 690.8 | P | 476.9 | P | 633.8 | P |
| | | ug = Mm.27578/len = 2764/STRA = for | | | | | | | |
| 7 | 96713_at | Cluster Incl AF052453 3-phosphoadenosine 5-phosphosulfate synthase 2/cds = (130,1995)/gb = AF052453/gi = 3342265/ | P | 201.8 | P | 49.9 | A | 116.9 | P |
| | | ug = Mm 27646/len = 1996/STRA = for | | | | | | | |
| 7 | 98942_r_at | Cluster Incl AW125284: UI-M-BH2 1-apy-a-11-0-UI s1 Mus musculus cDNA, 3 end/clone = UI-M-BH2 1-apy-a-11-0-UI/ | P | 209.7 | P | 63 | M | 122.3 | P |
| | | clone_end = 3/gb = AW125284/gi = 6100814/ug = Mm.28697/len = 336/STRA = for | | | | | | | |
| 7 | 96283_at | Cluster Incl AI849180 UI-M-AJ1-agy-f-10-0-UI s1 Mus musculus cDNA, 3 end/clone = UI-M-AJ1-agy-f-10-0-UI/ | P | 700.9 | P | 536.4 | P | 611.4 | A |
| | | clone_end = 3/gb = AI849180/gi = 5493086/ug = Mm.29870/len = 397/STRA = for | | | | | | | |
| 7 | 96930_at | Cluster Incl AI844128 UI-M-AL1-ahj-d-06-0-UI.s1 Mus musculus cDNA, 3 end/clone = UI-M-AL1-ahj-d-06-0-UI/ | P | 218.1 | P | 157.9 | P | 227.2 | P |
| | | clone_end = 3/gb = AI844128/gi = 5488034/ug = Mm.30169/len = 328/STRA = for | | | | | | | |
| 7 | 97444_at | Cluster Incl AI844520 UI-M-AJ1-ahf-a-08-0-UI s2 Mus musculus cDNA, 3 end/clone = UI-M-AJ1-ahf-a-08-0-UI/ | P | 333 | P | 216.6 | P | 297.3 | P |
| | | clone_end = 3/gb = AI844520/gi = 5488426/ug = Mm.30241/len = 491/STRA = for | | | | | | | |
| 7 | 98626_at | Cluster Incl AW047503 UI-M-BH1-ama-b-05-0-UI s1 Mus musculus cDNA, 3 end/clone = UI-M-BH1-ama-b-05-0-UI/ | P | 458.7 | P | 255.9 | M | 263.1 | P |
| | | clone_end = 3/gb = AW047503/gi = 5908032/ug = Mm.38746/len = 264/STRA = for | | | | | | | |
| 7 | 100592_at | Cluster Incl AI929971 uI60a06 y1 Mus musculus cDNA, 5 end/clone = IMAGE-2123410/clone_end = 5/gb = AI929971/ | P | 1086.1 | P | 711.3 | A | 1060.8 | P |
| | | gi = 5666012/ug = Mm 63/len = 510/STRA = rev | | | | | | | |
| 7 | 102378_at | Cluster Incl U02487: Kras oncogene-associated gene/cds = (27,677)/gb = U02487/gi = 437199/ug = Mm.1209/len = 4420/ | P | 63.5 | P | 74.7 | P | 83.4 | P |
| | | STRA = for | | | | | | | |
| 7 | 102387_at | Cluster Incl AA799042 vn40d08 r1 Mus musculus cDNA, 5 end/clone = IMAGE-1023663/clone_end = 5/gb = AA799042/ | P | 649.3 | P | 527.4 | P | 671.9 | A |
| | | gi = 2861997/ug = Mm.12183/len = 350/STRA = for | | | | | | | |
| 7 | 103032_at | Cluster Incl AF038008: Mus musculus tyrosylprotein sulfotransferase-1 mRNA, complete cds/cds = (210,1322)/ | P | 328.3 | P | 171.9 | P | 204.1 | P |
| | | gb = AF038008/gi = 3046915/ug = Mm.16084/len = 1867/STRA = for | | | | | | | |
| 7 | 104389_at | Cluster Incl AW049360 UI-M-BH1-ane-a-10-0-UI s1 Mus musculus cDNA, 3 end/clone = UI-M-BH1-ane-a-10-0-UI/ | P | 304.4 | P | 292.4 | P | 282.2 | P |
| | | clone_end = 3/gb = AW049360/gi = 5909889/ug = Mm.22712/len = 358/STRA = for | | | | | | | |
| 7 | 94384_at | Cluster Incl X67644 M musculus gly96 mRNA/cds = UNKNOWN/gb = X67644/gi = 287803/ug = Mm 25613/len = 1938/ | P | 456.2 | P | 477.9 | P | 398.7 | P |
| | | STRA = for | | | | | | | |
| 7 | 94948_at | Cluster Incl AF097511: Mus musculus zyxin related protein-1 (Zrp1) mRNA, complete cds/cds = (66,1508)/gb = AF097511/ | P | 333.6 | P | 358.5 | P | 273.2 | P |
| | | gi = 3851623/ug = Mm.27063/len = 1664/STRA = for | | | | | | | |
| 7 | 95037_at | Cluster Incl AW046639 UI-M-BH1-ald-d-10-0-UI s1 Mus musculus cDNA, 3 end/clone = UI-M-BH1-ald-d-10-0-UI/ | P | 377.7 | P | 168.6 | P | 447.4 | P |
| | | clone_end = 3/gb = AW046639/gi = 5907168/ug = Mm.27557/len = 338/STRA = for | | | | | | | |
| 7 | 96752_at | Cluster Incl M90551 Intercellular adhesion molecule/cds = (75,1688)/gb = M90551/gi = 459870/ug = Mm 28973/len = 2540/ | P | 176.4 | P | 179.5 | P | 137.1 | M |
| | | STRA = for | | | | | | | |
| 7 | 97404_at | Cluster Incl AW048244 UI-M-BH1-aly-b-04-0-UI.s1 Mus musculus cDNA, 3 end/clone = UI-M-BH1-aly-b-04-0-UI/ | P | 207.2 | P | 189.8 | P | 248.7 | P |
| | | clone_end = 3/gb = AW048244/gi = 5908773/ug = Mm 29901/len = 328/STRA = for | | | | | | | |
| 7 | 98988_at | Cluster Incl AA614971 Vo32e09 r1 Mus musculus cDNA, 5 end/clone = IMAGE-1051624/clone_end = 5/gb = AA614971/ | P | 242 | P | 101.1 | P | 128.9 | A |
| | | gi = 2502199/ug = Mm 3732/len = 137/STRA = for | | | | | | | |
| 7 | 100977_at | Cluster Incl AA691492 vu15h09 r1 Mus musculus cDNA, 5 end/clone = IMAGE-1180769/clone_end = 5/gb = AA691492/ | P | 261.7 | P | 189 | P | 255 | P |
| | | gi = 2692430/ug = Mm 6691/len = 449/STRA = for | | | | | | | |
| 7 | 101834_at | Cluster Incl Z14249 Protein kinase, mitogen activated kinase 3/cds = UNKNOWN/gb = Z14249/gi = 50858/ug = Mm 8385/ | P | 451 | P | 501.2 | P | 488.1 | P |
| | | len = 1718/STRA = for | | | | | | | |
| 7 | 103311_at | Cluster Incl AW121299 UI-M-BH2.3-aog-f-02-0-UI.s1 Mus musculus cDNA, 3 end/clone = UI-M-BH2.3-aog-f-02-0-UI/ | P | 426.2 | P | 351.1 | P | 412.6 | P |
| | | clone_end = 3/gb = AW121299/gi = 6096632/ug = Mm 21199/len = 236/STRA = for | | | | | | | |
| 7 | 104019_at | Cluster Incl AI851068 UI-M-BH0-ajv-h-07-UI s1 Mus musculus cDNA, 3 end/clone = UI-M-BH0-ajv-h-07-0-UI/ | P | 131 | P | 64 | P | 71.1 | A |
| | | clone end = 3/gb = AI851068/gi = 5494974/ug = Mm 26936/len = 441/STRA = for | | | | | | | |
| 7 | 100277_at | Cluster Incl X69619 lnhibin beta-A/cds = (172,1446)/gb = X69619/gi = 50145/ug = Mm 8042/len = 1529/STRA = for | P | 222.7 | P | 303.5 | P | 277.2 | M |
| 7 | 93062_at | Cluster Incl AW045881 UI-M-BH1-aky-h-01-0-UI.s1 Mus musculus cDNA, 3 end/clone = UI-M-BH1-aky-h-01-0-UI/ | P | 515.5 | P | 176 | A | 416.1 | P |
| | | clone end = 3/gb = AW045881/gi = 5906410/ug = Mm.1555/len = 561/STRA = for | | | | | | | |
| 7 | 93550_at | Cluster Incl D88792: Mouse mRNA for double LIM protein-1, complete cds/cds = (6,587)/gb = D88792/gi = 1695701/ | P | 363.1 | P | 184.7 | P | 303.5 | P |
| | | ug = Mm 2020/len = 822/STRA = for | | | | | | | |
| 7 | 96035_at | Cluster Incl L47335 Mus musculus branched chain alpha ketoacid decarboxylase E1a subunit mRNA, complete cds/ | P | 204.4 | P | 91.3 | A | 207.3 | P |
| | | cds = (17,1345)/gb = L47335/gi = 992564/ug = Mm 25848/len = 1681/STRA = for | | | | | | | |
| 7 | 95669_g_at | Cluster Incl AI840972 UI-M-AK0-adk-e-04-0-UI.s1 Mus musculus cDNA, 3 end/clone = UI-M-AK0-adk-e-04-0-UI/ | M | 354.1 | A | 344 | P | 330.2 | P |
| | | clone_end = 3/gb = AI840972/gi = 5475185/ug = Mm.29580/len = 456/STRA = for | | | | | | | |
| 7 | 96885_at | Cluster Incl AW122271.UI-M-BH2 2-aov-h-12-0-UI s1 Mus musculus cDNA, 3 end/clone = UI-M-BH2 2-aov-h-12-0-UI/ | A | 483.1 | P | 362.7 | P | 371.1 | P |
| | | clone_end = 3/gb = AW122271/gi = 6097746/ug = Mm 30083/len = 240/STRA = for | | | | | | | |
| 7 | 99167_at | Cluster Incl AW122550 UI-M-BH2.2-aox-d-08-0-UI.s1 Mus musculus cDNA, 3 end/clone = UI-M-BH2 2-aox-d-08-0-UI/ | P | 278.8 | P | 195.4 | A | 205.1 | P |
| | | clone_end = 3/gb = AW122550/gi_6098074/ug = Mm 41687/len = 462/STRA = for | | | | | | | |
| 7 | 103037_at | Cluster Incl U8366 Mus musculus cardiotrophin-1 mRNA complete cds/cds = (19,630)/gb = 18366)/gi = 710331 | P | 227.3 | P | 51.6 | P | 99 | M |
| | | ug = Mm 1616/len = 1345/stra = STRA = for | | | | | | | |
| 7 | 103044_g_at | Cluster Incl Z35294: M. musculus MTCP-1 gene/cds = (540,746)/gb = Z35294/gi = 603859/ug = Mm.16366/len = 954/ | P | 485.3 | P | 383.9 | P | 348.1 | A |
| | | STRA = for | | | | | | | |
| 7 | 103579_at | Cluster Incl X53247 M musculus EN-7 mRNA/cds = (36,614)/gb = X53247/gi = 50836/ug = Mm 1972/len = 1255/STRA = for | P | 669 | P | 536.8 | P | 815.6 | P |
| 7 | 104306_at | Cluster Incl AI847886 UI-M-AM1-afu-g-07-0-UI.s1 Mus musculus cDNA, 3 end/clone = UI-M-AM1-afu-g-07-0-UI/ | P | 339.1 | P | 288.3 | P | 311.8 | A |
| | | clone_end = 3/gb = AI847886/gi = 5491792/ug = Mm 22369/len = 392/STRA = for | | | | | | | |
| 7 | 94332_at | Cluster Incl AI882555 ub97f03 r1 Mus musculus cDNA, 5 end/clone = IMAGE-1396445/clone_end = 5/gb = AI882555/ | P | 113.1 | P | 69 | A | 102.9 | P |
| | | gi = 5567644/ug = Mm 2490/len = 329/STRA = for | | | | | | | |
| 7 | 94377_at | Cluster Incl AI854793: UI-M-BH0-aka-f-02-0-UI s1 Mus musculus cDNA, 3 end/clone = UI-M-BH0-aka-f-02-0-UI/ | P | 65.8 | P | −37.7 | A | 73.3 | P |
| | | clone_end = 3/gb = AI854793/gi = 5498699/ug = Mm 25547/len = 359/STRA = for | | | | | | | |
| 7 | 97353_at | Cluster Incl AI837497: UI-M-AL0-abr-g-08-0-UI s1 Mus musculus cDNA, 3 end/clone = UI-M-AL0-abr-g-08-0-UI/ | P | 156.6 | P | 92.6 | P | 135.3 | A |
| | | clone_end = 3/gb = AI837497/gi = 5471710/ug = Mm 29629/len = 224/STRA = for | | | | | | | |
| 7 | 98492_at | Cluster Incl AA920419 vz26a09 r1 Mus musculus cDNA, 5 end/clone = IMAGE-1327576/clone_end = 5/gb = AA920419/ | P | 730.1 | P | 799.3 | P | 604.6 | A |
| | | gi = 3067198/ug = Mm 35600/len = 371/STRA = for | | | | | | | |
| 7 | 99015_at | Cluster Incl U33626 Promyelocytic leukemia/cds = (86,2512)/gb = U33626/gi = 995828/ug = Mm 38497/len = 3760/STRA = for | P | 374.8 | P | 249.6 | P | 236.7 | A |
| 7 | 99444_at | Cluster Incl AJ250490.Mus musculus mRNA for receptor activity modifying protein 2 (Ramp2 gene)/cds = (30,599)/ | P | 241.9 | P | 107.6 | M | 263.1 | A |
| | | gb = AJ250490/gi = 6119626/ug = Mm 40358/len = 808/STRA = for | | | | | | | |
| 7 | 102736_at | Cluster Incl M19681 Small inducible cytokine A2/cds = (82,528)/gb = M19681/gi = 193486/ug = Mm.145/len = 798/STRA = for | P | 284.1 | P | −23.8 | A | −15.6 | A |
| 7 | 103690_at | Cluster Incl AW125574 UI-M-BH2.2-aqj-c-05-0-UI.s1 Mus musculus cDNA, 3 end/clone = UI-M-BH2.2-aqj-c-05-0-UI/ | P | 195.9 | P | 63.7 | A | 141.6 | A |
| | | clone_end = 3/gb = AW125574/gi = 6101104/ug = Mm 23955/len = 566/STRA = rev | | | | | | | |
| 7 | 93376_at | Cluster Incl AA673486 vp49f04 r1 Mus musculus cDNA, 3 end/clone = IMAGE-1080031/clone_end = 3/gb = AA673486/ | P | 146.5 | P | 51.6 | A | 60.6 | P |
| | | gi = 2646768/ug = Mm 6156/len = 477/STRA = rev | | | | | | | |
| 7 | 102114_f_at | Cluster Incl AI326963 mj76a06.x1 Mus musculus cDNA, 3 end/clone = IMAGE-482002/clone_end = 3/gb = AI326963/ | P | 435.2 | P | 185.7 | A | 194.1 | A |
| | | gi = 4061392/ug = Mm 42609/len = 479/STRA = rev | | | | | | | |
| 7 | 93301_at | Cluster Incl AW124624 UI-M-BH2 1-apw-b-05-0-UI s1 Mus musculus cDNA, 3 end/clone = UI-M-BH2 1-apw-b-05-0-UI/ | P | 109.3 | P | 130.8 | A | 80.1 | P |
| | | clone_end = 3/gb = AW124624/gi = 6100154/ug = Mm 18230/len = 416/STRA = for | | | | | | | |
| 7 | 93563_s_at | Cluster Incl AB017202: Mus musculus mRNA for entactin-2, complete cds/cds = (43,4254)/gb = AB017202/gi = 3513367/ | P | 177.4 | P | 74.1 | P | 53.5 | A |
| | | ug = Mm 20348/len = 5094/STRA = for | | | | | | | |
| 7 | 96618_at | Cluster Incl AW045637.UI-M-BH1-alg-f-02-0-UI s1 Mus musculus cDNA, 3 end/clone = UI-M-BH1-alg-f-02-0-UI/ | P | 149.9 | P | 40 | P | 40.3 | A |
| | | clone_end = 3/gb = AW045637/gi = 5906166/ug = Mm 27131/len = 371/STRA = for | | | | | | | |
| 7 | 96640_at | Cluster Incl AI644158 vw55c12 x1 Mus musculus cDNA, 3 end/clone = IMAGE-1247734/clone_end = 3/gb = AI644158/ | P | 423.6 | P | 295.2 | P | 310.6 | P |
| | | gi = 4722633/ug = Mm 27274/len = 458/STRA = for | | | | | | | |
| 7 | 96843_at | Cluster Incl AF068116 Mus musculus eIF4E-like protein 4E-LP mRNA, complete cds/cds = (0,737)/gb = AF068116/ | P | 203.5 | A | 259.1 | P | 377.3 | P |
| | | gi = 3201957/ug = Mm 3002/len = 804/STRA = for | | | | | | | |
| 7 | 99603_g_at | Cluster Incl AF064088 Mus musculus transcription factor GIF mRNA, complete cds/cds = (109,1548)/gb = AF064088/ | P | 332.7 | P | 205.8 | A | 180.9 | A |
| | | gi = 3139151/ug = Mm 4292/len = 3036/STRA = for | | | | | | | |
| 7 | 97360_at | Cluster Incl AI841689 UI-M-AO0-acg-h-11-0-UI s1 Mus musculus cDNA, 3 end/clone = UI-M-AO0-acg-h-11-0-UI/ | A | 139.4 | P | 78.5 | P | 114.2 | P |
| | | clone_end = 3/gb = AI841689/gi = 5475902/ug = Mm 29658/len = 337/STRA = for | | | | | | | |
| 7 | 99930_s_at | Cluster Incl U57325 Mus musculus PS-2short mRNA, complete cds/cds = (124,579)/gb = U57325/gi = 2315275/ | P | 84.3 | P | 14.3 | P | 109.9 | P |
| | | ug = Mm 4338/len = 1146/STRA = for | | | | | | | |
| 7 | 103308_at | Cluster Incl AI450597: mq87e05 x1 Mus musculus cDNA, 3 end/clone = IMAGE-585728/clone_end = 3/gb = AI450597/ | A | 645.3 | P | 439.6 | P | 872.7 | P |
| | | gi = 4298115/ug = Mm 21144/len = 350/STRA = for | | | | | | | |
| 7 | 93068_r_at | Cluster Incl U62674 Mus musculus histone H2a 2-615 (H2a-615), and histone H3.2-615 (H3-615) genes, complete cds/ | P | 399.6 | P | 467.2 | A | 407.4 | A |
| | | cds = (43,435)/gb = U62674/gi = 1458142/ug = Mm 15686/len = 515/STRA = for | | | | | | | |
| 7 | 93328_at | Cluster Incl X57437: Histidine decarboxylase cluster/cds = (74,2062)/gb = X57437/gi = 52903/ug = Mm 18603/len = 2371/ | P | 454.6 | P | −39.1 | A | 194.2 | A |
| | | STRA = for | | | | | | | |
| 7 | 94840_at | Cluster Incl U05837 Hexosaminidase A/cds = (150,1736)/gb = U05837/gi = 2182207/ug = Mm 2284/len = 1960/STRA = for | P | 281.7 | P | 109.5 | A | 163.5 | P |
| 7 | 103433_at | Cluster Incl AI846077 UI-M-AN1-afj-a-02-0-UI s2 Mus musculus cDNA, 3 end/clone = UI-M-AN1-afj-a-02-0-UI/ | P | 185.1 | P | 20.2 | A | 71.5 | M |
| | | clone_end = 3/gb = AI846077/gi = 5490023/ug = Mm 19065/len = 296/STRA = for | | | | | | | |
| 7 | 103563_at | Cluster Incl AW125713 UI-M-BH2 2-aql-c-12-0-UI s1 Mus musculus cDNA, 3 end/clone = UI-M-BH2 2-aql-c-12-0-UI/ | P | 51.7 | P | −58.3 | A | −79.9 | A |
| | | clone_end = 3/gb = AW125713/gi = 6101283/ug = Mm 19126/len = 445/STRA = for | | | | | | | |
| 7 | 104516_at | Cluster Incl U82758 Mus musculus lung-specific membrane protein mRNA, complete cds/cds = (21,677)/gb = U82758/ | A | 204.6 | P | 31.5 | A | 217.5 | P |
| | | gi = 3342229/ug = Mm 22768/len = 1319/STRA = for | | | | | | | |
| 7 | 102926_at | Cluster Incl AB008833 Mus musculus mRNA for GFRalpha-3, complete cds/cds = (45,1238)/gb = AB008833/gi = 2627159/ | A | 404.3 | P | 307.6 | P | 287.9 | P |
| | | ug = Mm.16520/len = 1867/STRA = for | | | | | | | |
| 7 | 92731_at | Cluster Incl X83601: Pentaxin related gene/cds = (99,1244)/gb = X83601/gi = 1181243/ug = Mm 4663/len = 1841/STRA = for | P | 57.2 | P | 11 | A | 35.8 | A |
| 7 | 94761_at | Cluster Incl X70058: Small Inducible cytokine A7/cds = (70,342)/gb = X70058/gi = 437872/ug = Mm 16091/len = 774/ | P | 196.8 | P | 14.4 | A | 103.8 | A |
| | | STRA = for | | | | | | | |
| 7 | 102100_f_at | Cluster Incl AI845835: UI-M-AO1-aeg-d-01-0-UI s1 Mus musculus cDNA, 3 end/clone = UI-M-AO1-aeg-d-01-0-UI/ | A | 45.1 | P | 17.6 | A | 37.2 | A |
| | | clone_end = 3/gb = AI845835/gi = 5489741/ug = Mm 42439/len = 269/STRA = rev | | | | | | | |
| 7 | 100521_at | Cluster Incl D32137 Mouse mRNA for oviducl-specific glycoprotein (MOGP), complete cds/cds = (14,2179)/gb = D32137/ | M | 91.9 | P | 66.1 | M | 40.3 | A |
| | | gi = 806523/ug = Mm 5110/len = 2525/STRA = rev | | | | | | | |
| 7 | 96131_at | Cluster Incl AI840116: UI-M-AL0-abt-h-05-0-UI s1 Mus musculus cDNA, 3 end/clone = UI-M-AL0-abt-h-05-0-UI/ | P | 309 | P | 171.3 | M | 230.1 | M |
| | | clone_end = 3/gb = AI840116/gi = 5474329/ug = Mm.28140/len = 348/STRA = for | | | | | | | |
| 7 | 104508_at | Cluster Incl AA756506 vv53h12 r1 Mus musculus cDNA, 5 end/clone = IMAGE-1226183/clone_end = 5/gb = AA756506/ | P | 143.1 | P | 126.5 | A | 225.5 | A |
| | | gi = 2803704/ug = Mm 30805/len = 473/STRA = for | | | | | | | |
| 7 | 104647_at | Cluster Incl M88242: Mouse glucocortoid-regulated inflammatory prostaglandin G/H synthase (griPGHS) mRNA, complete | P | 70.6 | P | 23.9 | A | 23.1 | A |
| | | cds/cds = (100,1914)/gb = M88242/gi = 193637/ug = Mm 3137/len = 4152/STRA = for | | | | | | | |
| 7 | 93153_at | Cluster Incl AW124433: UI-M-BH2.1-apo-b-10-0-UI.s1 Mus musculus cDNA, 3 end/clone = UI-M-BH2.1-apo-b-10-0-UI/ | P | 215.6 | A | 99.9 | M | 130.4 | A |
| | | clone_end = 3/gb = AW124433/gi = 6100003/ug = Mm.5258/len = 526/STRA = rev | | | | | | | |
| 7 | 95956_at | Cluster Incl C76923: C76923 Mus musculus cDNA, 3 end/clone = J0023A02/clone_end = 3/gb = C76923/gi = 2979405/ | A | 79.7 | A | 94.9 | P | 99.9 | P |
| | | ug = Mm 24933/len = 429/STRA = rev | | | | | | | |
| 7 | 97103_at | Cluster Incl AF031380 Mus musculus KOI-4 gene, partial cds/cds = (0,102)/gb = AF031380/gi = 2623675/ug = Mm 32715/ | P | −69.9 | A | −83.9 | A | −193.2 | A |
| | | len = 103/STRA = rev | | | | | | | |
| 7 | 92549_at | Cluster Incl U97170 Mus musculus protein kinase inhibitor gamma (PKIg) mRNA, complete cds/cds = (217,447)/ | A | 373.3 | P | 254.1 | A | 499.7 | A |
| | | gb = U97170/gi = 3004635/ug = Mm 10091/len = 1064/STRA = for | | | | | | | |
| 7 | 93985_at | Cluster Incl AW120868: UI-M-BH2.3-aoa-g-07-0-UI s1 Mus musculus cDNA, 3 end/clone = UI-M-BH2.3-aoa-g-07-0-UI/ | P | 37.5 | P | −117.9 | A | −140.3 | A |
| | | clone_end = 3/gb = AW120868/gi = 6096201/ug = Mm 21712/len = 284/STRA = for | | | | | | | |
| 7 | 96605_at | Cluster Incl AI787183: u86a11 y1 Mus musculus cDNA, 5 end/clone = IMAGE-1889276/clone_end = 5/gb = AI787183/ | P | 102.6 | P | 62 | A | −3.4 | M |
| | | gi = 5334899/ug = Mm 27061/len = 245/STRA = for | | | | | | | |
| 7 | 97811_at | Cluster Incl AI844507: UI-M-AJ1-ahe-g-11-0-UI s1 Mus musculus cDNA, 3 end/clone = UI-M-AJ1-ahe-g-11-0-UI/ | P | 4.9 | P | −43.7 | A | −40.1 | M |
| | | clone_end = 3/gb = AI844507/gi = 5488413/ug = Mm 28163/len = 299/STRA = for | | | | | | | |
| 7 | 94517_r_at | Cluster Incl M55181 Preproenkephalin 2/cds = UNKNOWN/gb = M55181/gi = 201032/ug = Mm 2899/len = 1408/STRA = for | P | 556.8 | P | 219.7 | A | 508.5 | A |
| 7 | 102326_at | Cluster Incl AB002664 Mus musculus mRNA for p67phox, complete cds/cds = (96,1673)/gb = AB002664/gi = 3061283/ | P | 98.5 | P | 38.6 | A | 87.5 | A |
| | | ug = Mm.10729/len = 1813/STRA = for | | | | | | | |
| 7 | 96203_at | Cluster Incl AI847646.UI-M-AP1-agg-g-07-0-UI s1 Mus musculus cDNA, 3 end/clone = UI-M-AP1-agg-g-07-0-UI/ | M | 60.2 | A | 17 | A | 42.1 | A |
| | | clone_end = 3/gb = AI847646/gi = 5491552/ug = Mm 28623/len = 226/STRA = for | | | | | | | |
| 7 | 99486_at | Cluster Incl X55038 Centromere autoantigen B/cds = (0,1799)/gb = X55038/gi = 50381/ug = Mm 41454/len = 1800/STRA = for | A | 206.1 | P | 124.1 | A | 107.7 | A |
| 7 | 92534_at | Cluster Incl U10551 GTP binding protein (gene overexpressed in skeletal muscle)/cds = (108,995)/gb = U10551/gi = 511464/ | P | 61.3 | A | −5.7 | A | 19.8 | A |
| | | ug = Mm 4362/len = 1981/STRA = for | | | | | | | |
| 7 | 92688_at | Cluster Incl X57199 Acid phosphatase 2, lysosomal/cds = (0,1265)/gb = X57199/gi = 52870/ug = Mm 45570/len = 1266/ | M | 187.6 | P | 148.2 | M | 175 | A |
| | | STRA = for | | | | | | | |
| 7 | 93656_g_at | Cluster Incl X95316 M musculus USF1 (exons 2 to 10)/cds = (214,1146)/gb = X95316/gi = 1765921/ug = Mm 8/len = 1902/ | A | 242.6 | A | 235.2 | A | 290.4 | A |
| | | STRA = for | | | | | | | |
| 7 | 94778_at | Cluster Incl U96401: Mus musculus aldehyde dehydrogenase Ahd-2-like mRNA, complete cds/cds = (44,1549)/gb = U96401/ | M | 98.5 | A | 125.1 | A | 116.5 | P |
| | | gi = 2289239/ug = Mm.16773/len = 2060/STRA = for | | | | | | | |
| 7 | 95380_at | Cluster Incl L19057 Non MHC restricted killing associated/cds = (126,1322)/gb = L19057/gi = 309076/ug = Mm 2299/ | A | 40.5 | A | 35.8 | A | 45.6 | A |
| | | len = 1930/STRA = for | | | | | | | |
| 7 | 99835_at | Cluster Incl AF017128 Fos-like antigen 1/cds = (253,1074)/gb = AF017128/gi = 2407190/ug = Mm 6215/len = 1705/STRA = for | P | −3.1 | A | −85.2 | A | 113 | A |
| 8 | 92574_at | Cluster Incl AI851046 UI-M-BH0-ajv-f-05-0-UI s1 Mus musculus cDNA, 3 end/clone = UI-M-BH0-ajv-f-05-0-UI/ | P | 1182 | P | 887.5 | P | 844.2 | P |
| | | clone_end = 3/gb = AI851046/gi = 5494952/ug = Mm 10406/len = 475/STRA = for | | | | | | | |
| 8 | 92615_at | Cluster Incl AI853615 UI-M-BH0-ajp-f-09-0-UI s1 Mus musculus cDNA, 3 end/clone = UI-M-BH0-ajp-f-09-0-UI/ | P | 1178.8 | P | 936 | P | 847.3 | P |
| | | clone_end = 3/gb = AI853615/gi = 5497521/ug = Mm 1103/len = 542/STRA = for | | | | | | | |
| 8 | 92625_at | Cluster Incl X68193: M. musculus mRNA for nucleoside diphoshate kinase B/cds = (19,477)/gb = X68193/gi = 53353/ | P | 2912 | P | 4077.9 | P | 3644.7 | P |
| | | ug = Mm.1125/len = 612/STRA = for | | | | | | | |
| 8 | 93026_at | Cluster Incl AW124337.UI-M-BH2 1-apq-f-08-0-UI.s1 Mus musculus cDNA, 3 end/clone = UI-M-BH2.1-apq-f-08-0-UI/ | P | 1337 | P | 992.2 | P | 667.9 | P |
| | | clone_end = 3/gb = AW124337/gi = 6099867/ug = Mm 14796/len = 243/STRA = rev | | | | | | | |
| 8 | 93071_at | Cluster Incl X99644: M musculus mRNA for TIF1 beta protein/cds = (168,2672)/gb = X99644/gi = 1480113/ug = Mm 15701/ | P | 1800.2 | P | 2105.2 | P | 2031.5 | P |
| | | len = 2858/STRA = for | | | | | | | |
| 8 | 93270_at | Cluster Incl AI839918 UI-M-AH0-acw-f-01-0-UI s1 Mus musculus cDNA, 3 end/clone = UI-M-AH0-acw-f-01-0-UI/ | P | 811.5 | P | 858.2 | P | 929.8 | P |
| | | clone_end = 3/gb = AI839918/gi = 5474131/ug = Mm 17403/len = 429/STRA = for | | | | | | | |
| 8 | 93295_at | Cluster Incl Z31555 M musculus (129/Sv) Ccte mRNA for CCT (chaperonin containing TCP-1) epsilon subunit/ | P | 1821.9 | P | 2176 | P | 1915.6 | P |
| | | cds = (99,1724)/gb = Z31555/gi = 468549/ug = Mm 1813/len = 1769/STRA = for | | | | | | | |
| 8 | 93305_f_at | Cluster Incl AF053724: Mus musculus endobrevin mRNA, complete cds/cds = (154,459)/gb = AF053724/gi = 2982367/ | P | 651.9 | P | 726 | P | 743 | P |
| | | ug = Mm 1838/len = 597/STRA = for | | | | | | | |
| 8 | 93340_f_at | Cluster Incl AF043120: Mus musculus beta prime coatomer protein mRNA, partial cds/cds = (0,1352)/gb = AF043120/ | P | 345 | P | 312.6 | P | 337.4 | P |
| | | gi = 2809536/ug = Mm.18729/len = 1615/STRA = for | | | | | | | |
| 8 | 93358_at | Cluster Incl AI836451 UI-M-AI0-aak-e-05-0-UI.s1 Mus musculus cDNA, 3 end/clone = UI-M-AI0-aak-e-05-0-UI/ | P | 1025 | P | 1098.6 | P | 960.8 | P |
| | | clone_end = 3/gb = AI836451/gi = 5470664/ug = Mm.18924/len = 368/STRA = for | | | | | | | |
| 8 | 93533_at | Cluster Incl AI853651 UI-M-BH0-ajq-b-03-0-UI s1 Mus musculus cDNA, 3 end/clone = UI-M-BH0-ajq-b-03-0-UI/ | P | 584.7 | P | 473.4 | P | 398.4 | P |
| | | clone_end = 3/gb = AI853651/gi = 5497557/ug = Mm 1985/len = 445/STRA = for | | | | | | | |
| 8 | 93557_at | Cluster Incl U43285 Selenophosphate synthetase 2/cds = (137,1495)/gb = U43285/gi = 1815623/ug = Mm.20294/len = 2202/ | P | 217.1 | P | 200.4 | P | 190.1 | P |
| | | STRA = for | | | | | | | |
| 8 | 93581_at | Cluster Incl AI845121 UI-M-BG0-ahs-b-12-0-UI.s1 Mus musculus cDNA, 3 end/clone = UI-M-BG0-ahs-b-12-0-UI/ | P | 1949.1 | P | 2148.8 | P | 1909.4 | P |
| | | clone_end = 3/gb = AI845121/gi = 5489027/ug = Mm 2060/len = 361/STRA = for | | | | | | | |
| 8 | 93593_f_at | Cluster Incl U87948 Epithelial membrane protein 3/cds = (201,692)/gb = U87948/gi = 2358253/ug = Mm 20829/len = 695/ | P | 1460.7 | P | 1287.4 | P | 1260.2 | P |
| | | STRA = for | | | | | | | |
| 8 | 93596_i_at | Cluster Incl AA624586 vn82e11.r1 Mus musculus cDNA, 5 end/clone = IMAGE-1038476/clone_end = 5/gb = AA624588/ | P | 1164.5 | P | 1660.8 | P | 956.5 | P |
| | | gi = 2528462/ug = Mm 20841/len = 178/STRA = for | | | | | | | |
| 8 | 93734_i_at | Cluster Incl D49686: Mus musculus mRNA for Tat binding protein-1, complete cds/cds = (176,1504)/gb = D49686/ | P | 558.6 | P | 588.8 | P | 684 | P |
| | | gi = 3510276/ug = Mm.20946/len = 1569/STRA = for | | | | | | | |
| 8 | 93735_f_at | Cluster Incl D49686: Mus musculus mRNA for Tat binding protein-1, complete cds/cds = (176,1504)/gb = D49686/ | P | 1260.6 | P | 1261.1 | P | 1113.7 | P |
| | | gi = 3510276/ug = Mm.20946/len = 1569/STRA = for | | | | | | | |
| 8 | 93760_at | Cluster Incl AI838759 UI-M-AO0-acf-f-02-0-UI s1 Mus musculus cDNA, 3 end/clone = UI-M-AO0-acf-f-02-0-UI/ | P | 963.3 | P | 919.6 | P | 1014.5 | P |
| | | clone_end = 3/gb = AI838759/gi = 5472972/ug = Mm.21149/len = 460/STRA = for | | | | | | | |
| 8 | 93774_at | Cluster Incl AW049040 UI-M-BH1-amp-h-08-0-UI s1 Mus musculus cDNA, 3 end/clone = UI-M-BH1-amp-h-08-0-UI/ | P | 859.4 | P | 705.8 | P | 644.5 | P |
| | | clone_end = 3/gb = AW049040/gi = 5909569/ug = Mm.21207/len = 260/STRA = for | | | | | | | |
| 8 | 93789_a_at | Cluster Incl AF038848: Mus musculus transcriptional repressor SIN3B (Sin3B) mRNA, short form, complete cds/ | P | 1104.7 | P | 855.8 | P | 987.3 | P |
| | | cds = (20,901)/gb = AF038848/gi = 2921546/ug = Mm.2137/len = 1056/STRA = for | | | | | | | |
| 8 | 93823_at | Cluster Incl AI844864: UI-M-AH1-agx-d-05-0-UI.s1 Mus musculus cDNA, 3 end/clone = UI-M-AH1-agx-d-05-0-UI/ | P | 168.5 | P | 182.7 | P | 137.3 | P |
| | | clone_end = 3/gb = AI844864/gi = 5488735/ug = Mm 21534/len = 527/STRA = for | | | | | | | |
| 8 | 93968_at | Cluster Incl AA870917: vq28a06.r1 Mus musculus cDNA, 5 end/clone = IMAGE-1095538/clone_end = 5/gb = AA870917/ | P | 933.1 | P | 851.5 | P | 714.5 | P |
| | | gi = 2968382/ug = Mm 21650/len = 454/STRA = for | | | | | | | |
| 8 | 93972_at | Cluster Incl AI8374493 UI-M-AL0-abr-f-12-0-UI.s1 Mus musculus cDNA, 3 end/clone = UI-M-AL0-abr-f-12-0-UI/clone_end = 3 | P | 1571 | P | 1752.1 | P | 1539.6 | P |
| | | gb = AI83749/gi = 5471706/ug = Mm.21669/len = 341/STRA = for | | | | | | | |
| 8 | 93973_at | Cluster Incl AI854624 UI-M-BH0-ake-e-06-0-UI.a1 Mus musculus cDNA, 3 end/clone = UI-M-BH0-ake-e-06-0-UI/ | P | 836.8 | P | 1222.1 | P | 1050.7 | P |
| | | clone_end = 3/gb = AI854624/gi = 5498530/ug = Mm 21671/len = 474/STRA = for | | | | | | | |
| 8 | 93988_at | Cluster Incl AI836676 UI-M-AP0-abh-g-06-0-UI a1 Mus musculus cDNA, 3 end/clone = UI-M-AP0-abh-g-06-0-UI/ | P | 1119.6 | P | 1381.7 | P | 1214.2 | P |
| | | clone_end = 3/gb = AI836676/gi = 5470889/ug = Mm 21728/len = 471/STRA = for | | | | | | | |
| 8 | 94001_at | Cluster Incl U65735 Mus musculus eiav G homolog (MelG) mRNA, complete cds/cds = (0,980)/gb = U65735/gi = 1655900/ | P | 975.7 | P | 1222.3 | P | 556.3 | P |
| | | ug = Mm 21766/len = 981/STRA = for | | | | | | | |
| 8 | 94004_at | Cluster Incl Z19543: M musculus h2-calponin cDNA/cds = (102,1019)/gb = Z51143/ug = Mm 21776/len = 1821/ | P | 526.8 | P | 563.6 | P | 394.6 | P |
| | | STRA = for | | | | | | | |
| 8 | 94025_at | Cluster Incl AW045339: UI-M-BH1-ako-a-09-0-UI s1 Mus musculus cDNA, 3 end/clone = UI-M-BH1-ako-a-09-0-UI/ | P | 1149 | P | 1645.7 | P | 700.4 | P |
| | | clone_end = 3/gb = AW045339/gi = 5905868/ug = Mm 21874/len = 430/STRA = for | | | | | | | |
| 8 | 94061_at | Cluster Incl M13018 Cysteine rich Intestinal protein/cds = (61,294)/gb = M13018/gi = 192725/ug = Mm 22049/len = 404/ | P | 1170.8 | P | 694.9 | P | 749.4 | P |
| | | STRA = for | | | | | | | |
| 8 | 94062_at | Cluster Incl AI847609: UI-M-AP1-agg-c-11-0-UI s1 Mus musculus cDNA, 3 end/clone = UI-M-AP1-agg-c-11-0-UI/ | P | 2214.9 | P | 2146.3 | P | 2052.4 | P |
| | | clone_end = 3/gb = AI847609/gi = 5491515/ug = Mm 2206/len = 356/STRA = for | | | | | | | |
| 8 | 94078_at | Cluster Incl AW046239: UI-M-BH1-ala-b-12-0-UI s1 Mus musculus cDNA, 3 end/clone = UI-M-BH1-ala-b-12-0-UI/ | P | 2262.5 | P | 2243 | P | 2098 | P |
| | | clone_end = 3/gb = AW046239/gi = 5906768/ug = Mm 22138/len = 363/STRA = for | | | | | | | |
| 8 | 94219_at | Cluster Incl AI853269: UI-M-BH0-ajl-c-11-0-UI s1 Mus musculus cDNA, 3 end/clone = UI-M-BH0-ajl-c-11-0-UI/clone_end = 3/ | P | 1065.8 | P | 1614.2 | P | 1021.8 | P |
| | | gb = AI853269/gi = 5479175/ug = Mm 22233/len = 375/STRA = for | | | | | | | |
| 8 | 94263_f_at | Cluster Incl D85570 Proteasome (prosome, macropain) subunit, beta type 7/cds = (20,853)/gb = D85570/gi = 2547069/ | P | 1885.4 | P | 2117.8 | P | 1832.4 | P |
| | | ug = Mm 2246/len = 991/STRA = for | | | | | | | |
| 8 | 94821_at | Cluster Incl AW123880 UI-M-BH2.1-app-e-07-0-UI.s1 Mus musculus cDNA, 3 end/clone = UI-M-BH2.1-app-e-07-0-UI/ | P | 1064.5 | P | 1139.3 | P | 1023.6 | P |
| | | clone_end = 3/gb = W123880/gi = 6099410/ug = Mm 22718/len = 448/STRA = for | | | | | | | |
| 8 | 94841_at | Cluster Incl AW148997 UI-M-BH1-amp-c-10-0-UI s1 Mus musculus cDNA, 3 end/clone = UI-M-BH1-amp-c-10-0-UI/ | P | 1123.6 | P | 1146.1 | P | 965.1 | P |
| | | clone_end = 3/gb = AW048997/gi = 5909526/ug = Mm 2287/len = 486/STRA = for | | | | | | | |
| 8 | 94875_at | Cluster Incl AI838915 UI-M-AO0-ace-a-11-0-UI s1 Mus musculus cDNA, 3 end/clone = UI-M-AO0-ace-a-11-0-UI/ | P | 462.6 | P | 351.7 | P | 357.9 | P |
| | | clone_end = 3/gb = AI838915/gi = 5473128/ug = Mm 23825/len = 398/STRA = for | | | | | | | |
| 8 | 94876_f_at | Cluster Incl AI849207: UI-M-AJ1-agz-a-02-0-UI s1 Mus musculus cDNA, 3 end/clone = UI-M-AJ1-agz-a-02-0-UI/ | P | 761.5 | P | 809.6 | P | 538.6 | P |
| | | clone_end = 3/gb = AI849207/gi = 5493113/ug = Mm 23836/len = 449/STRA = for | | | | | | | |
| 8 | 94897_at | Cluster Incl D87896 Glutathione peroxidase 4/cds = (144,737)/gb = D87896/gi = 2522258/ug = Mm 2400/len = 958/STRA = for | P | 641.7 | P | 681.3 | P | 730.7 | P |
| 8 | 94907_f_at | Cluster Incl AW045632: UI-M-BH1-alg-e-06-0-UI s1 Mus musculus cDNA, 3 end/clone = UI-M-BH1-alg-e-06-0-UI/ | P | 1262.6 | P | 1356.1 | P | 1038.9 | P |
| | | clone _end = 3/gb = AW045632/gi = 5906161/ug = Mm 24091/len = 248/STRA = for | | | | | | | |
| 8 | 94909_at | Cluster Incl AW046919: UI-M-BH1-akl-g-03-0-UI s1 Mus musculus cDNA, 3 end/clone = UI-M-BH1-akl-g-03-0-UI/ | P | 502.2 | P | 360.1 | P | 362.6 | P |
| | | clone _end = 3/gb = AW046919/gi = 5907448/ug = Mm 24104/len = 440/STRA = for | | | | | | | |
| 8 | 94912_at | Cluster Incl AI835592: UI-M-AI0-app-a-05-0-UI.s1 Mus musculus cDNA, 3 end/clone = UI-M-AI0-aap-a-05-0-UI/ | P | 782 | P | 804.9 | P | 531.8 | P |
| | | clone_end = 3/gb = AI835592/gi = 5469805/ug = Mm 24108/len = 229/STRA = for | | | | | | | |
| 8 | 94913_at | Cluster Incl U65313 Mus musculus ras-GTPase-activating protein SH3-domain binding protein (G3BP) mRNA, complete | P | 497.2 | P | 488.7 | P | 453.2 | P |
| | | cds/cds = (164,1612)/gb = U65313/gi = 2961438/ug = Mm.2411/len = 1800/STRA = for | | | | | | | |
| 8 | 95409_at | Cluster Incl AW047671: UI-M-BH1-alo-c-06-0-UI s1 Mus musculus cDNA, 3 end/clone = UI-M-BH1-alo-c-06-0-UI/ | P | 628 | P | 548.6 | P | 522.8 | P |
| | | clone_end = 3/gb = AW047671/gi = 5908200/ug = Mm 24228/len = 339/STRA = for | | | | | | | |
| 8 | 95424_at | Cluster Incl AW120986: UI-M-BH2 3-aod-f-01-0-UI s1 Mus musculus cDNA, 3 end/clone = UI-M-BH2.3-aod-f-01-0-UI/ | P | 2974.2 | P | 2970.1 | P | 2763.9 | P |
| | | clone_end = 3/gb = AW120986/gi = 609631/ug = Mm 24433/len = 498/STRA = for | | | | | | | |
| 8 | 95441_at | Cluster Incl AB201122: Mus musculus Tim23 mRNA for translocase of inner mitochondrial membrane, complete cds/ | P | 1997.8 | P | 2559.4 | P | 2635 | P |
| | | cds = (123,752)/gb = AB021122/gi = 4996327/ug = Mm 24565/len = 1142/STRA = for | | | | | | | |
| 8 | 95462_at | Cluster Incl AW060951 UI-M-BH1-anv-f-08-0-UI s1 Mus musculus cDNA, 3 end/clone = UI-M-BH1-anv-f-08-0-UI/ | P | 1084.7 | P | 1097.7 | P | 882.5 | P |
| | | clone_end = 3/gb = AW060951/gi = 6008702/ug = Mm 24745/len = 228/STRA = for | | | | | | | |
| 8 | 95517_i_at | Cluster Incl AI846338: UI-M-AN1-afa-c-01-0-UI.s1 Mus musculus cDNA, 3 end/clone = UI-M-AN1-afa-c-10-0-UI/ | P | 733.6 | P | 899.8 | P | 783.2 | P |
| | | clone_end = 3/gb = AI846338/gi = 5490244/ug = Mm 25308/len = 377/STRA = for | | | | | | | |
| 8 | 96008_at | Cluster Incl U81052: Mus musculus Defender against Apoptotic Death (Dad1) gene/cds = (25,366)/gb = U81052/gi = 1905978/ | P | 1261.9 | P | 1454.8 | P | 1408.2 | P |
| | | ug = Mm 2547/len = 406/STRA = for | | | | | | | |
| 8 | 96611_at | Cluster Incl AI843074 UI-M-AK1-aes-s-11-0-UI s1 Mus musculus cDNA, 3 end/clone = UI-M-AK1-aes-a-11-0-UI/ | P | 953.1 | P | 1037.8 | P | 547.1 | P |
| | | clone_end = 3/gb = AI843074/gi = 5477287/ug = Mm 27087/len = 253/STRA = for | | | | | | | |
| 8 | 96666_at | Cluster Incl U57692 N-terminal Asn amidase/cds = (33,965)/gb = U57692/gi = 1373366/ug = Mm 27378/len = 1101/STRA = for | P | 45.4 | P | 181.1 | P | 27.7 | P |
| 8 | 96686_i_at | Cluster Incl AI853864: UI-M-BH0-ajr-f-08-0-UI s1 Mus musculus cDNA, 3 end/clone = UI-M-BH0-ajr-f-08-0-UI/clone_end = 3/ | P | 1950.5 | P | 2051.3 | P | 1423.4 | P |
| | | gb = AI853864/gi = 5497770 ug = Mm.27477/len = 461/STRA = for | | | | | | | |
| 8 | 96687_f_at | Cluster Incl AI853864 UI-M-BH0-ajr-f-08-0-UI s1 Mus musculus cDNA, 3 end/clone = UI-M-BH0-ajr-f-08-0-UI/clone_end = 3/ | P | 829.2 | P | 912.2 | P | 735.5 | P |
| | | gb = AI853864/gi = 5497770 ug = Mm 27477/len = 461/STRA = for | | | | | | | |
| 8 | 97203_at | Cluster Incl X61399: Mouse F52 mRNA for a novel protein/cds = UNKNOWN/gb = X61399/gi = 50943/ug = Mm 2769/ | P | 1235.8 | P | 1558.5 | P | 1247.4 | P |
| | | len = 1584/STRA = for | | | | | | | |
| 8 | 97204_s_at | Cluster Incl AI850983: UI-M-BH0-akd-h-08-0-UI s1 Mus musculus cDNA, 3 end/clone = UI-M-BH0-akd-h-08-0-UI/ | P | 281.7 | P | 288.1 | P | 231.9 | P |
| | | clone_end = 3/gb = AI850983/gi = 5494889/ug = Mm 27692/len = 265/STRA = for | | | | | | | |
| 8 | 97207_f_at | Cluster Incl AI875934: uj64h06 x1 Mus musculus cDNA, 3 end/clone = IMAGE-1924763/clone_end = 3/gb = AI675934/ | P | 2139.7 | P | 2165.6 | P | 2079.7 | P |
| | | gi = 5549983/ug = Mm 27700/len = 330/STRA = for | | | | | | | |
| 8 | 97248_at | Cluster Incl X61431: M. musculus mRNA for diazepam-binding inhibitor/cds = (62,325)/gb = X61431/gi = 50712/ug = Mm.2785/ | P | 2457.1 | P | 2376.7 | P | 1808 | P |
| | | len = 390/STRA = for | | | | | | | |
| 8 | 97911_at | Cluster Incl AI835950-UI-M-AI0-aao-f-11-0-UI.s1 Mus musculus cDNA. 3 end/clone = UI-M-AI0-aao-f-11-0-UI/clone_end = 3/ | P | 562.7 | P | 655.5 | P | 629.9 | P |
| | | gb = AI835950/gi = 5470163/ug = Mm 28482/len = 380/STRA = for | | | | | | | |
| 8 | 98875_at | Cluster Incl AW121160: UI-M-BH2 3-aoh-g-08-0-UI s1 Mus musculus cDNA, 3 end/clone = UI-M-BH2 3-aoh-g-08-0-UI/ | P | 98 | P | 152.4 | P | 82.8 | P |
| | | clone_end = 3/gb = AW121160/gi = 6096493/ug = Mm 28492/len = 139/STRA = for | | | | | | | |
| 8 | 94494 _at | Cluster Incl AI840856 UI-M-AH0-adb-g-11-0-UI s1 Mus musculus cDNA, 3 end/clone = UI-M-AH0-adb-g-11-0-UI/ | P | 357 | P | 422.2 | P | 274.7 | P |
| | | clone_end = 3/gb = AI840856/gi = 5475069/ug = Mm 28922/len = 274/STRA = for | | | | | | | |
| 8 | 94506 _at | Cluster Incl AI853113 UI-M-BH0-ajf-d-10-0-UI s1 Mus musculus cDNA, 3 end/clone = UI-M-BH0-ajf-d-10-0-UI/ | P | 314.2 | P | 397.5 | P | 259.9 | P |
| | | clone_end = 3/gb = AI853113/gi = 5497019/ug = Mm.28961/len = 205/STRA = for | | | | | | | |
| 8 | 94522 _at | Cluster Incl AF098508 Mus musculus dynaclin light chain (P24) mRNA, complete cds/cds = (12,572)/gb = AF098508/ | P | 633.9 | P | 657.7 | P | 620.5 | P |
| | | gi = 4323147/ug = Mm.29025/len = 819/STRA = for | | | | | | | |
| 8 | 94550 _at | Cluster Incl AW121324 UI-M-BH2.2-aom-a-06-0-UI s1 Mus musculus cDNA, 3 end/clone = UI-M-BH2.2-aom-a-06-0-UI/ | P | 612.4 | P | 507.9 | P | 448 | P |
| | | clone_end = 3/gb = AW121324/gi = 6096657/ug = Mm 29079/len = 541/STRA = for | | | | | | | |
| 8 | 95042 _at | Cluster Incl AI839770 UI-M-AN0-acq-f-01-0-UI s1 Mus musculus cDNA, 3 end/clone = UI-M-AN0-acq-f-01-0-UI/ | P | 579.5 | P | 484.5 | P | 434.7 | P |
| | | clone_end = 3/gb = AI839770/gi = 5474023/ug = Mm 29118/len = 148/STRA = for | | | | | | | |
| 8 | 95066 _at | Cluster Incl U67611 Mouse transaidolase gene mRNA, complete cds/cds = (5,1018)/gb = U67611/gi = 2589165/ | P | 398 | P | 438.7 | P | 207.5 | P |
| | | ug = Mm 29132/len = 1155/STRA = for | | | | | | | |
| 8 | 95091_at | Cluster Incl AI839895.UI-M-AH0-acw-d-02-0-UI s1 Mus musculus cDNA, 3 end/clone = UI-M-AH0-acw-d-02-0-UI/ | P | 713.6 | P | 666.7 | P | 510 | P |
| | | clone_end = 3/gb = AI839895/gi = 5474108/ug = Mm 29296/len = 446/STRA = for | | | | | | | |
| 8 | 95097 _at | Cluster Incl AI851227: UI-M-BH0-ajx-h-07-0-UI s1 Mus musculus cDNA, 3 end/clone = UI-M-BH0-ajx-h-07-0-UI/ | P | 596.3 | P | 642.1 | P | 602.3 | P |
| | | clone_end = 3/gb = AI851227/gi = 5495133/ug = Mm 29317/len = 354/STRA = for | | | | | | | |
| 8 | 95131_f_at | Cluster Incl AI852592: UI-M-BH0-aiu-b-11-0-UI s1 Mus musculus cDNA, 3 end/clone = UI-M-BH0-aiu-b-11-0-UI/ | P | 1908 | P | 2336 | P | 1794.6 | P |
| | | clone_end = 3/gb = AI852592/gi = 5496498/ug = Mm 29415/len = 392/STRA = rev | | | | | | | |
| 8 | 95137 _at | Cluster Incl AI852985: UI-M-BH0-ajn-a-04-0-UI s1 Mus musculus cDNA, 3 end/clone = UI-M-BH0-ajn-a-04-0-UI/ | P | 545.3 | P | 793.4 | P | 583.3 | P |
| | | clone_end = 3/gb = AI852985/gi = 5496891/ug = Mm 29431/len = 428/STRA = for | | | | | | | |
| 8 | 95147 _at | Clusler Incl AI843795: UI-M-AN1-afc-e-05-0-UI.s1 Mus musculus cDNA, 3 end/clone = UI-M-AN1-afc-e-05-0-UI/ | P | 1480 | P | 2059.7 | P | 1620.5 | P |
| | | clone_end = 3/gb = AI843795/gi = 5478008/ug = Mm 29456/len = 462/STRA = for | | | | | | | |
| 8 | 95149_at | Cluster Incl AW121088: UI-M-BH2 3-aof-h-04-0-UI s1 Mus musculus cDNA, 3 end/clone = UI-M-BH2.3-aof-h-04-0-UI/ | P | 823.1 | P | 831.7 | P | 538.9 | P |
| | | clone_end = 3/gb = AW121088/gi = 6096421/ug = Mm 29473/len = 505/STRA = for | | | | | | | |
| 8 | 95659_at | Cluster Incl AF041476 Mus musculus BAF53a (Baf53a) mRNA, complete cds/cds = (130,1419)/gb = AF041476/gi = 4001804/ | P | 429.9 | P | 443.2 | P | 408.9 | P |
| | | ug = Mm 29546/len = 1770/STRA = for | | | | | | | |
| 8 | 95690_at | Cluster Incl AW047363 UI-M-BH1-ame-c-09-0-UI s1 Mus musculus cDNA, 3 end/clone = UI-M-BH1-ame-c-09-0-UI/ | P | 561.5 | P | 503.8 | P | 404.9 | P |
| | | clone_end = 3/gb = AW047363/gi = 5907892/ug = Mm 29651/len = 398/STRA = for | | | | | | | |
| 8 | 95696_at | Cluster Incl AI840882: UI-M-AM0-adm-b-04-0-UI.s1 Mus musculus cDNA, 3 end/clone = UI-M-AM0-adm-b-04-0-UI/ | P | 1485.7 | P | 2096.7 | P | 1813.5 | P |
| | | clone_end = 3/gb = AI840882/gi = 5475095/ug = Mm 29675/len = 400/STRA = for | | | | | | | |
| 8 | 95700_f_at | Cluster Incl AI848094 UI-M-AP1-agk-d-05-0-UI s1 Mus musculus cDNA, 3 end/clone = UI-M-AP1-agk-d-05-0-UI/ | P | 322.3 | P | 344.3 | P | 355.6 | P |
| | | clone_end = 3/gb = AI848094/gi = 5492000/ug = Mm 29685/len = 399/STRA = for | | | | | | | |
| 8 | 95703_at | Cluster Incl AB024303 Mus musculus mRNA similar to human Sua1, complete cds/cds = (34,1086)/gb = AB024303/ | P | 666.2 | P | 663.6 | P | 545.7 | P |
| | | gi = 5689241/ug = Mm 29698/len = 1870/STRA = for | | | | | | | |
| 8 | 95707_at | Cluster Incl AA615853 Vo94104.r1 Mus musculus cDNA, 5 end/clone = IMAGE-1066783/clone_end = 5/gb = AA615853/ | P | 503.9 | P | 711.6 | P | 633.3 | P |
| | | gi = 2503081/ug = Mm 29700/len = 534/STRA = for | | | | | | | |
| 8 | 95714_at | Cluster Incl AI226264: uf06a12y1 Mus musculus cDNA, 5 end/clone = IMAGE-1510558/clone_end = 5/gb = AI226264/ | P | 803.2 | P | 780.6 | P | 569.4 | P |
| | | gi = 3809317/ug = Mm 29712/len = 465/STRA = for | | | | | | | |
| 8 | 95716_at | Cluster Incl AW125041: UI-M-BH2 1-apx-b-01-0-UI s1 Mus musculus cDNA, 3 end/clone = UI-M-BH2 1-apx-b-01-0-UI/ | P | 2567.7 | P | 2939.1 | P | 2923.6 | P |
| | | clone_end = 3/gb = AW125041/gi = 6100571/ug = Mm 29717/len = 227/STRA = for | | | | | | | |
| 8 | 95718_f_at | Cluster Incl AW060158 UI-M-BH1-amu-b-10-0-UI s1 Mus musculus cDNA, 3 end/clone = UI-M-BH1-amu-b-10-0-UI/ | P | 2542.5 | P | 3273.7 | P | 2550.9 | P |
| | | clone_end = 3/gb = AW060158/gi = 6007924/ug = Mm 29722/len = 360/STRA = for | | | | | | | |
| 8 | 95736_at | Cluster Incl AI847546: UI-M-AM1-afv-f-01-0-UI s1 Mus musculus cDNA, 3 end/clone = UI-M-AM1-afv-f-01-0-UI/ | P | 669.5 | P | 780.6 | P | 474.6 | P |
| | | clone_end = 3/gb = AI847546/gi = 5491452/ug = Mm 29749/len = 452/STRA = for | | | | | | | |
| 8 | 95742_at | Cluster Incl AW045451: UI-M-BH1-akr-g-10-0-UI s1 Mus musculus cDNA, 3 end/clone = UI-M-BH1-akr-g-10-0-UI/ | P | 456.8 | P | 615.7 | P | 470.5 | P |
| | | clone_end = 3/gb = AW045451/gi = 5906020/ug = Mm 29760/len = 340/STRA = for | | | | | | | |
| 8 | 95760_at | Cluster Incl AI848730: UI-M-AJ1-ahf-d-04-0-UI.s1 Mus musculus cDNA, 3 end/clone = UI-M-AJ1-ahf-d-04-0-UI/ | P | 364.6 | P | 375.7 | P | 322.4 | P |
| | | clone_end = 3/gb = AI848730/gi = 5492636/ug = Mm 29801/len = 477/STRA = for | | | | | | | |
| 8 | 96249_at | Cluster Incl AW122105: UI-M-BH2 2-aoo-h-12-0-UI.s1 Mus musculus cDNA, 3 end/clone = UI-M-BH2.2-aoo-h-12-0-UI/ | P | 904.4 | P | 574.1 | P | 606.7 | P |
| | | clone_end = 3/gb = AW122105/gi = 6097518/ug = Mm.29812/len = 357/STRA = rev | | | | | | | |
| 8 | 96258_at | Cluster Incl AI843448. UI-M-AQ1-aec-e-01-0-UI s1 Mus musculus cDNA, 3 end/clone = UI-M-AQ1-aec-e-01-0-UI/ | P | 1397.6 | P | 2153.5 | P | 1603.6 | P |
| | | clone_end = 3/gb = AI843448/gi = 5477661/ug = Mm 29823/len = 382/STRA = for | | | | | | | |
| 8 | 96276_r_at | Cluster Incl AI843327: UI-M-AQ1-adz-a-05-0-UI s1 Mus musculus cDNA, 3 end/clone = UI-M-AQ1-adz-a-05-0-UI/ | P | 260.4 | P | 280.9 | P | 328.9 | P |
| | | clone_end = 3/gb = AI843327/gi = 5477540/ug = Mm 2986/len = 339/STRA = for | | | | | | | |
| 8 | 96291_f_at | Cluster Incl AI835847 UI-M-AI0-aat-b-07-0-UI s1 Mus musculus cDNA, 3 end/clone = UI-M-AI0-aat-b-07-0-UI/clone_end = 3/ | P | 1201.3 | P | 1349 | P | 1088.2 | P |
| | | gb = AI835847/gi = 5470060/ug = Mm 29897/len = 333/STRA = for | | | | | | | |
| 8 | 96292_r_at | Cluster Incl AI835847 UI-M-AI0-aat-b-07-0-UI s1 Mus musculus cDNA, 3 end/clone = UI-M-AI0-aat-b-07-0-UI/clone_end = 3/ | P | 1814.2 | P | 2108 | P | 1433.7 | P |
| | | gb = AI835847/gi = 5470060/ug = Mm 29897/len = 333/STRA = for | | | | | | | |
| 8 | 96305_at | Cluster Incl AI845854.UI-M-AO1-aeg-e-10-0-UI s1 Mus musculus cDNA, 3 end/clone = UI-M-AO1-aeg-e-10-0-UI/ | P | 403.1 | P | 369.7 | P | 387.4 | P |
| | | clone_end = 3/gb = AI845854/gi = 5489760/ug = Mm 29916/len = 441/STRA = for | | | | | | | |
| 8 | 96321_at | Cluster Incl AI846396: UI-M-AN1-afa-h-08-0-UI s1 Mus musculus cDNA, 3 end/clone = UI-M-AN1-afa-h-08-0-UI/ | P | 1291.7 | P | 1761.7 | P | 1414.9 | P |
| | | clone_end = 3/gb = AI846396/gi = 5490302/ug = Mm 29939/len = 380/STRA = for | | | | | | | |
| 8 | 96329_at | Cluster Incl AI837793.UI-M-AK0-adl-e-09-0-UI s1 Mus musculus cDNA, 3 end/clone = UI-M-AK0-adl-e-09-0-UI/ | P | 529.3 | P | 427.6 | P | 397.6 | P |
| | | clone_end = 3/gb = AI837793/gi = 5472006/ug = Mm 29965/len = 480/STRA = for | | | | | | | |
| 8 | 96341_at | Cluster Incl AI849280 UI-M-AJ1-agz-h-05-0-UI.s1 Mus musculus cDNA, 3 end/clone = UI-M-AJ1-agz-h-05-0-UI/ | P | 301 | P | 327.3 | P | 303.8 | P |
| | | clone_end = 3/gb = AI849280/gi = 5493186/ug = Mm 29989/len = 520/STRA = for | | | | | | | |
| 8 | 96342_at | Cluster Incl AI846320: UI-M-AN1 -afa-a-04-0-U1.s1 Mus musculus cDNA, 3 end/clone = UI-M-AN1-afa-a-04-0-UI/ | P | 381 | P | 350.5 | P | 350.1 | P |
| | | clone_end = 3/gb = AI846320/gi = 5490226/ug = Mm.29990/len = 281/STRA = for | | | | | | | |
| 8 | 96360_at | Cluster Incl AW125498: UI-M-BH2.2-aqm-d-11-0-UI.s1 Mus musculus cDNA, 3 end/clone = UI-M-BH2.2-aqm-d-11-0-UI/ | P | 1779.2 | P | 2452.7 | P | 1950.9 | P |
| | | clone_end = 3/gb = AW125498/gi = 6101028/ug = Mm 30016/len = 389/STRA = for | | | | | | | |
| 8 | 96855_at | Cluster Incl AI839946: UI-M-AHO-acs-a-04-0-UI.s1 Mus musculus cDNA, 3 end/clone = UI-M-AH0-acs-a-04-0-UI/ | P | 698.2 | P | 897.3 | P | 722.9 | P |
| | | clone_end = 3/gb = AI839946/gi = 5474159/ug = Mm 30043/len = 255/STRA = for | | | | | | | |
| 8 | 96883_at | Cluster Incl U70733 Mus musculus translation initiation factor eIF3-p44 mRNA, complete cds/cds = (17,979)/gb = U70733/ | P | 926.2 | P | 1050.5 | P | 955 | P |
| | | gi = 4097872/ug = Mm 30080/len = 1025/STRA = for | | | | | | | |
| 8 | 96952_at | Cluster Incl AW121552 UI-M-BH2 2-aop-f-02-0-UI s1 Mus musculus cDNA, 3 end/clone = UI-M-BH2 2-aop-f-02-0-UI/ | P | 2101.2 | P | 2287.8 | P | 1812.6 | P |
| | | clone_end = 3/gb = AW121552/gi = 6096885/ug = Mm 30210/len = 521/STRA = for | | | | | | | |
| 8 | 97443_at | Cluster Incl AI850850 UI-M-BH0-ajt-c-05-0-UI s1 Mus musculus cDNA, 3 end/clone = UI-M-BH0-ajt-c-05-0-UI/clone_end = 3/ | P | 1459.3 | P | 1602.9 | P | 1420.4 | P |
| | | gb = AI850850/gi = 5494756/ug = Mm 30240/len = 321/STRA = for | | | | | | | |
| 8 | 97528_at | Cluster Incl AF022811 Mus musculus cornichon mRNA, complete cds/cds = (85,519)/gb = AF022811/gi = 3114215/ | P | 637.7 | P | 629.8 | P | 588.3 | P |
| | | ug = Mm 3261/len = 1388/STRA = for | | | | | | | |
| 8 | 98104_at | Cluster Incl AI842889 UI-M-AN1-afb-g-01-0-UI s1 Mus musculus cDNA, 3 end/clone = UI-M-AN1-alb-g-01-0-UI/ | P | 924.6 | P | 787.3 | P | 844 | P |
| | | clone_end = 3/gb = AI842889/gi = 5477102/ug = Mm 3479/len = 304/STRA = for | | | | | | | |
| 8 | 98545_at | Cluster Incl X78683 B-cell receptor-associated protein 37/cds = (107,1003)/gb = X78683/gi = 541733/ug = Mm 36241/ | P | 1507 | P | 1663.3 | P | 1799.4 | P |
| | | len = 1287/STRA = for | | | | | | | |
| 8 | 98575_at | Cluster Incl X13135 Fatty acid synthase/cds = (0,2516)/gb = X13135/gi = 50947/ug = Mm 3760/len = 3295/STRA = for | P | 907.5 | P | 1576.1 | P | 913.9 | P |
| 8 | 98618_at | Cluster Incl AA624336 vn01g09.r1 Mus musculus cDNA, 5 end/clone = IMAGE-1006528/clone_end = 5/gb = AA624336/ | P | 473.1 | P | 523.5 | P | 300.8 | P |
| | | gi = 2528212/ug = Mm 3856/len = 474/STRA = for | | | | | | | |
| 8 | 98631_g_at | Cluster Incl AW106745 um33b04 y1 Mus musculus cDNA, 5 end/clone = IMAGE-2236303/clone_end = 5/gb = AW106745/ | P | 293.5 | P | 292.9 | P | 256.8 | P |
| | | gi = 6077545/ug = Mm 38792/len = 600/STRA = for | | | | | | | |
| 8 | 99104_at | Cluster Incl U49915.Mus musculus adipocyte specific protein adipoQ (adipoQ) mRNA, complete cds/cds = (16,759)/ | P | 3232.9 | P | 3139.1 | P | 2042.8 | P |
| | | gb = U49915/gi = 1399497/ug = Mm 3969/len = 1152/STRA = for | | | | | | | |
| 8 | 99108_s_at | Cluster Incl U15012 Growth hormone receptor/cds = (0,430)/gb = U15012/gi = 666863/ug = Mm 3986/len = 513/STRA = for | P | 316.7 | P | 196.4 | P | 342.7 | P |
| 8 | 99140_at | Cluster Incl AW 124920 UI-M-BH2 1-apu-f-09-0-UI s1 Mus musculus cDNA, 3 end/clone = UI-M-BH2 1-apu-f-09-0-UI/ | P | 341 | P | 354.8 | P | 288.3 | P |
| | | clone_end = 3/gb = AW124920/gi = 6100450/ug = Mm 41390/len = 426/STRA = for | | | | | | | |
| 8 | 99150_at | Cluster Incl AI844357.UI-M-AL1-ahp-a-10-0-UI.s1 Mus musculus cDNA, 3 end/clone = UI-M-AL1-ahp-a-10-0-UI/ | P | 443.5 | P | 512.6 | P | 512.7 | P |
| | | clone_end = 3/gb = AI844357/gi = 5488263/ug = Mm 41541/len = 413/STRA = for | | | | | | | |
| 8 | 99564_at | Cluster Incl D87908 Mus musculus mRNA for nuclear protein np95, complete cds/cds = (148,2496)/gb = D87908/ | P | 438.7 | P | 420.3 | P | 316.2 | P |
| | | gi = 4220589/ug = Mm 42196/len = 3488/STRA = for | | | | | | | |
| 8 | 99581_at | Cluster Incl U60001 Mus musculus protein kinase C inhibitor (mPKCI) mRNA, complete cds/cds = (22,402)/gb = U60001/ | P | 3012.7 | P | 3041.5 | P | 3247.9 | P |
| | | gi = 1519045/ug = Mm 425/len = 544/STRA = for | | | | | | | |
| 8 | 99597_at | Cluster Incl AI841629 UI-M-AO0-acg-c-01-0-UI s1 Mus musculus cDNA, 3 end/clone = UI-M-AO0-acg-c-01-0-UI/ | P | 543.7 | P | 758.1 | P | 622.7 | P |
| | | clone_end = 3/gb = AI841629/gi = 5475842/ug = Mm 4284/len = 436/STRA = for | | | | | | | |
| 8 | 100079_at | Cluster Incl AI845556 UI-M-AO1-aek-h-05-0-UI.s1 Mus musculus cDNA, 3 end/clone = UI-M-AO1-aek-h-05-0-UI/ | P | 1667.3 | P | 1362.9 | P | 1198.1 | P |
| | | clone_end = 3/gb = AI845556/gi = 5489462/ug = Mm 4537/len = 428/STRA = for | | | | | | | |
| 8 | 100081_at | Cluster Incl U27830 Mus musculus mSTI1 mRNA, complete cds/cds = (55,1686)/gb = U27830/gi = 881484/ug = Mm 4540/ | P | 490.2 | P | 615.8 | P | 363.7 | P |
| | | len = 2080/STRA = for | | | | | | | |
| 8 | 100101_at | Cluster Incl L15447.Mouse small nuclear RNA (Rnu1a-1) mRNA, complete cds/cds = (99,962)/gb = L15447/gi = 349003/ | P | 582.5 | P | 550.2 | P | 459.2 | P |
| | | ug = Mm 4633/len = 1176/STRA = for | | | | | | | |
| 8 | 100527_at | Cluster Incl AW124744 UI-M-BH2.1-aqa-d-10-0-UI s1 Mus musculus cDNA, 3 end/clone = UI-M-BH2 1-aqa-d-10-0-UI/ | P | 478.8 | P | 473.9 | P | 396.4 | P |
| | | clone_end = 3/gb = AW124744/gi = 6100274/ug = Mm.52/len = 485/STRA = for | | | | | | | |
| 8 | 100575_at | Cluster Incl AI841645 UI-M-AO0-acg-d-06-0-UI s1 Mus musculus cDNA, 3 end/clone = UI-M-AO0-acg-d-06-0-UI/ | P | 258.9 | P | 407.7 | P | 204.1 | P |
| | | clone_end = 3/gb = AI841645/gi = 5475858/ug = Mm 5934/len = 256/STRA = for | | | | | | | |
| 8 | 101016_at | Cluster Incl D87898 Mus musculus mRNA for ARF1, complete cds/cds = (78,623)/gb = D87898/gi = 1565206/ug = Mm.6836/ | P | 3918 | P | 4258.8 | P | 4244.9 | P |
| | | len = 1032/STRA = for | | | | | | | |
| 8 | 101045_at | Cluster Incl U96116: Mus musculus amyloid beta-peptide binding protein (ERAB) mRNA, complete cds/cds = (104,898)/ | P | 543.1 | P | 775.1 | P | 745.5 | P |
| | | gb = U96116/gi = 2078283/ug = Mm.6994/len = 1029/STRA = for | | | | | | | |
| 8 | 101085_at | Cluster Incl AI854257 UI-M-BH0-ajs-b-02-0-UI s1 Mus musculus cDNA, 3 end/clone = UI-M-BH0-ajs-b-02-0-UI/ | P | 684.3 | P | 631.9 | P | 738.4 | P |
| | | clone_end = 3/gb = AI854257/gi = 5498163/ug = Mm.7328/len = 511/STRA = for | | | | | | | |
| 8 | 101112_g_at | Cluster Incl AF014371: Mus musculus Rho family GTPase (ArhA) mRNA, complete cds/cds = (134,715)/gb = AF014371/ | P | 875.7 | P | 1326.4 | P | 810.3 | P |
| | | gi = 3237319/ug = Mm.757/len = 815/STRA = for | | | | | | | |
| 8 | 101510_at | Cluster Incl AB007136 Protease (prosome, macropain) 28 subunit, alpha/cds = (81,830)/gb = AB007136/gi = 3219281/ | P | 797.7 | P | 586.7 | P | 584.9 | P |
| | | ug = Mm 830/len = 977/STRA = for | | | | | | | |
| 8 | 101525_at | Cluster Incl AI848871 UI-M-AJ1-ahb-f-06-0-UI.s1 Mus musculus cDNA 3 end/clone = UI-M-AJ1-ahb-f-06-0-UI/ | P | 878.5 | P | 1107.9 | P | 892.2 | P |
| | | clone_end = 3/gb = AI848871/gi = 5492777/ug = Mm.8688/len = 353/STRA = for | | | | | | | |
| 8 | 101558_s_at | Cluster Incl AB003304 Proteasome beta type subunit 5/cds = (7,801)/gb = AB003304/gi = 2897805/ug = Mm 8911/len = 874/ | P | 1196.7 | P | 1314.7 | P | 1063.3 | P |
| | | STRA = for | | | | | | | |
| 8 | 101954_at | Cluster Incl U70494 Mus musculus histone H2A Z (H2A Z) mRNA, complete cds/cds = (95,481)/gb = U70494/gi = 1575712/ | P | 2929.3 | P | 3898.4 | P | 2578.7 | P |
| | | ug = Mm 916/len = 989/STRA = for | | | | | | | |
| 8 | 101961_at | Cluster Incl U67327.Mus musculus WD40-repeat type I transmembrane protein A72.5 mRNA, complete cds/ | P | 1355.5 | P | 1370.8 | P | 906.1 | P |
| | | cds = (291,1031)/gb = U67327/gi = 1762983/ug = Mm 927/len = 1336/STRA = for | | | | | | | |
| 8 | 101992_at | Cluster Incl U13393: Mus musculus delta proteasome subunit mRNA, complete cds/cds = (69,677)/gb = U13393/gi = 984937/ | P | 979.6 | P | 1106.6 | P | 497 | P |
| | | ug = Mm 98/len = 721/STRA = for | | | | | | | |
| 8 | 102047_at | Cluster Incl AF043326 Mus musculus N-myristoyltransferase 1 mRNA complete cds/cds = (22,1512)/gb = AF043326/ | P | 616.5 | P | 698.5 | P | 428.3 | P |
| | | gi = 3005066/ug = Mm 10265/len = 1548/STRA = for | | | | | | | |
| 8 | 102821_s_at | Cluster Incl L32752 Mouse (clone M2) GTPase (Ran) mRNA, complete cds/cds = (37,687)/gb = L32752/gi = 727168/ | P | 2619.1 | P | 3117.4 | P | 1514.1 | P |
| | | ug = Mm 14426/len = 970/STRA = for | | | | | | | |
| 8 | 103350_at | Cluster Incl M64641: Moloney leukemia virus 34/cds = (78,1043)/gb = M64641/gi = 199770/ug = Mm.18347/len = 1547/ | P | 1042 | P | 1075.7 | P | 711.4 | P |
| | | STRA = for | | | | | | | |
| 8 | 103371_at | Cluster Incl AF100956: Mus musculus major histocompatibility locus class II region; Fas-binding protein Daxx (DAXX) gene, | P | 706.3 | P | 763.5 | P | 619.5 | P |
| | | partial cds; Bing1 (BING1), tapasin (tapasin), RalGDS-like factor (RLF), KE2 (KE2), BING4 (BING4), beta1, 3-galactosyl | | | | | | | |
| | | transferase (be/STRA = rev | | | | | | | |
| 8 | 103630_at | Cluster Incl AI844089 UI-M-AL1-ahi-h-12-0-UI s1 Mus musculus cDNA, 3 end/clone = UI-M-AL1-ahi-h-12-0-UI/ | P | 427.5 | P | 432.2 | P | 397 | P |
| | | clone_end = 3/gb = AI844089/gi = 5487995/ug = Mm.20290/len = 241/STRA = for | | | | | | | |
| 8 | 103821_at | Cluster Incl AJ223087: Mus musculus mRNA for Cdc6-related protein/cds = (103,1872)/gb = AJ223087/gi = 4007644/ | P | 159.8 | P | 222.7 | P | 100.6 | P |
| | | ug = Mm 20912/len = 2589/STRA = for | | | | | | | |
| 8 | 103910_at | Cluster Incl AJ249987: Mus musculus TAFII30 gene for mTAFII30 protein, exons 1-5/cds = (25,681)/gb = AJ249987/ | P | 666.8 | P | 710.5 | P | 546.8 | P |
| | | gi = 6119622/ug = Mm.21419/len = 759/STRA = for | | | | | | | |
| 8 | 104057_at | Cluster Incl AW012807 uo03a03 y1 Mus musculus cDNA, 5 end/clone = IMAGE-2582284/clone_end = 5/gb = AW012807/ | P | 306 | P | 308.1 | P | 205.2 | P |
| | | gi = 5861585/ug = Mm 21535/len = 544/STRA = for | | | | | | | |
| 8 | 104279_at | Cluster Incl AW125116 UI-M-BH2.1-apx-h-12-0-UI s1 Mus musculus cDNA, 3 end/clone = UI-M-BH2.1-apx-h-12-0-UI/ | P | 1310.7 | P | 1504.4 | P | 1501.3 | P |
| | | clone_end = 3/gb = AW125116/gi = 6100646/ug = Mm 22230/len = 442/STRA = for | | | | | | | |
| 8 | 104371_at | Cluster Incl AF078752 Mus musculus diacylglycerol acyltransferase (Dgat) mRNA, complete cds/cds = (95,1591)/ | P | 537.9 | P | 452.4 | P | 434.6 | P |
| | | gb = AF078752/gi = 3859933/ug = Mm 22633/len = 1650/STRA = for | | | | | | | |
| 8 | 104560_at | Cluster Incl AW125634: UI-M-BH2.2-aqj-h-11-0-UI s1 Mus musculus cDNA, 3 end/clone = UI-M-BH2.2-aqj-h-11-0-UI/ | P | 316.1 | P | 262 | P | 403.1 | P |
| | | clone_end = 3/gb = AW125634/gi = 6101164/ug = Mm 23827/len = 447/STRA = for | | | | | | | |
| 8 | 104573_at | Cluster Incl AA921069 vy83h09.r1 Mus musculus cDNA, 5 end/clone = IMAGE-1312865/clone_end = 5/gb = AA921069/ | P | 420.5 | P | 533.3 | P | 307.5 | P |
| | | gi = 3067848/ug = Mm 23925/len = 263/STRA = for | | | | | | | |
| 8 | 104633_at | Cluster Incl AW123921. UI-M-BH2.3-aqc-a-07-0-UI s1 Mus musculus cDNA, 3 end/clone = UI-M-BH2 3-aqc-a-07-0-UI/ | P | 413.5 | P | 265.1 | P | 338.3 | P |
| | | clone_end = 3/gb = AW123921/gi = 6099451/ug = Mm 24342/len = 236/STRA = for | | | | | | | |
| 8 | 94327_at | Cluster Incl AW230209 up28c10 y1 Mus musculus cDNA, 5 end/clone = IMAGE-2655666/clone_end = 5/gb = AW230209/ | P | 437.4 | P | 453.2 | P | 412.5 | P |
| | | gi = 6559505/ug = Mm 24875/len = 259/STRA = for | | | | | | | |
| 8 | 94340_at | Cluster Incl AW124224 UI-M-BH2.1-aph-d-08-0-UI s1 Mus musculus cDNA, 3 end/clone = UI-M-BH2.1-aph-d-08-0-UI/ | P | 364.9 | P | 299.6 | P | 302.4 | P |
| | | clone_end = 3/gb = AW124224/gi = 6099754/ug = Mm 25042/len = 383/STRA = for | | | | | | | |
| 8 | 95631_at | Cluster Incl AF088911: Mus musculus protein phosphatase X (Ppx) mRNA, complete cds/cds = (139,1062)/gb = AF088911/ | P | 1442.2 | P | 1680.3 | P | 1486.4 | P |
| | | gi = 4028534/ug = Mm 28082/len = 1276/STRA = rev | | | | | | | |
| 8 | 96122_at | Cluster Incl AW049373 UI-M-BH1-ane-b-11-0-UI s1 Mus musculus cDNA, 3 end/clone = UI-M-BH1-ane-b-11-0-UI/ | P | 351.1 | P | 212 | P | 396.6 | P |
| | | clone_end = 3/gb = AW049373/gi = 5909902/ug = Mm.28108/len = 580/STRA = for | | | | | | | |
| 8 | 96237_at | Cluster Incl AI118905 uc15f04 r1 Mus musculus cDNA, 5 end/clone = IMAGE-1398079/clone_end = 5/gb = AI118905/ | P | 424.2 | P | 383.7 | P | 185.6 | P |
| | | gi = 3519229/ug = Mm 28800/len = 311/STRA = for | | | | | | | |
| 8 | 96834_at | Cluster Incl AI843586 UI-M-AO1-aeh-f-08-0-UI s1 Mus musculus cDNA, 3 end/clone = UI-M-AO1-aeh-f-08-0-UI/ | P | 1041.1 | P | 1269.2 | P | 942.2 | P |
| | | clone_end = 3/gb = AI843586/gi = 5477764/ug = Mm 29426/len = 398/STRA = for | | | | | | | |
| 8 | 97342_at | Cluster Incl AW060867 UI-M-BH1-amx-f-08-0-UI s1 Mus musculus cDNA, 3 end/clone = UI-M-BH1-amx-f-08-0-UI/ | P | 1105.9 | P | 1127.8 | P | 767.6 | P |
| | | clone_end = 3/gb = AW060867/gi = 6008618/ug = Mm 29599/len = 418/STRA = for | | | | | | | |
| 8 | 98446_s_at | Cluster Incl U06834 Mus musculus Balb/c eph-related receptor protein tyrosine kinase mRNA, complete cds/ | P | 524.4 | P | 545 | P | 472.7 | P |
| | | cds = (102,3065)/gb = U06834/gi = 459172/ug = Mm 34533/len = 3733/STRA = rev | | | | | | | |
| 8 | 98495_at | Cluster Incl AI846906.UI-M-AK1-aey-b-11-0-UI.s1 Mus musculus cDNA, 3 end/clone = UI-M-AK1-aey-b-11-0-UI/ | P | 524.9 | P | 375.8 | P | 383.9 | P |
| | | clone_end = 3/gb = AI846908/gi = 5490812/ug = Mm 3561/len = 336/STRA = for | | | | | | | |
| 8 | 100007_at | Cluster Incl AI837573 UI-M-AL0-abs-g-06-0-UI.s1 Mus musculus cDNA, 3 end/clone = UI-M-AL0-abs-g-06-0-UI/ | P | 825.1 | P | 1107.2 | P | 971.5 | P |
| | | clone_end = 3/gb-AI837573/gi = 5471786/ug = Mm 4532/len = 293/STRA = for | | | | | | | |
| 8 | 100959_at | Cluster Incl X99921.M musculus mRNA for S100 calcium-binding protein A13/cds = (60,356)/gb = X99921/gi = 1694840/ | P | 1318 | P | 1396.6 | P | 1171.9 | P |
| | | ug = Mm.6523/len = 440/STRA = for | | | | | | | |
| 8 | 100964_at | Cluster Incl AF035208 Mus musculus putative v-SNARE Vti1b mRNA, complete cds/cds = (11,709)/gb = AF035208/ | P | 781.5 | P | 852.6 | P | 840.8 | P |
| | | gi = 3213228/ug = Mm 6577/len = 909/STRA = for | | | | | | | |
| 8 | 100970_at | Cluster Incl X65687: Thymoma viral proto-oncogene/cds = (283,1725)/gb = X65687/gi = 287806/ug = Mm 6845/len = 2626/ | P | 763.7 | P | 805.5 | P | 628.5 | P |
| | | STRA = for | | | | | | | |
| 8 | 100990_g_at | Cluster Incl AJ001373 Mus musculus bodenin gene/cds = (173,775)/gb = AJ001373/gi = 2647955/ug = Mm 6818/len = 954/ | P | 383.5 | P | 360 | P | 311.3 | P |
| | | STRA = for | | | | | | | |
| 8 | 101449_at | Cluster Incl AW120841 UI-M-BH2.3-aoa-e-02-0-UI s1 Mus musculus cDNA, 3 end/clone = UI-M-BH2.3-aoa-e-02-0-UI/ | P | 181.5 | P | 192.1 | P | 164.7 | P |
| | | clone_end = 3/gb = AW120841/gi = 6096174/ug = Mm 794/len = 283/STRA = for | | | | | | | |
| 8 | 101461_f_at | Cluster Incl U06944 Praja1/cds = (328,1602)/gb = U06944/gi = 2828789/ug = Mm 8211/len = 1978/STRA = for | P | 1610.6 | P | 1547.1 | P | 1506.4 | P |
| 8 | 101905_at | Cluster Incl AF037454: lichy/cds = (149,2713)/gb = AF037454/gi = 2827197/ug = Mm 8975/len = 5122/STRA = for | P | 1342.8 | P | 1784.7 | P | 1357.8 | P |
| 8 | 101931_at | Cluster Incl AF042731: Mus musculus phosphatidylserine synthase-1 mRNA, complete cds/cds = (93,1514)/gb = AF042731/ | P | 783.5 | P | 914.7 | P | 785 | P |
| | | gi = 2811227/ug = Mm 9440/len = 2455/STRA = for | | | | | | | |
| 8 | 102235_at | Cluster Incl X13945 Lung carcinoma myc related oncogene 1/cds = (0,1106)/gb = X13945/gi = 53287/ug = Mm.1055/ | P | 370.5 | P | 470.4 | P | 374 | P |
| | | len = 3076/STRA = for | | | | | | | |
| 8 | 94105_at | Cluster Incl L78075: Cell division cycle 42/cds = (0,575)/gb = L78075/gi = 1321598/ug = Mm 1022/len = 576/STRA = for | P | 1479.5 | P | 1476.3 | P | 1274.1 | P |
| 8 | 100345_f_at | Cluster Incl W65964: me10a08 r1 Mus musculus cDNA, 5 and/clone = IMAGE-387062/clone_end = 5/gb = W65964/ | P | 1622.3 | P | 1773.8 | P | 1627.8 | P |
| | | gi = 1373784/ug = Mm.88247/len = 404/STRA = rev | | | | | | | |
| 8 | 97060_at | Cluster Incl AW215489 up04f11 x1 Mus musculus cDNA, 3 end/clone = IMAGE-2651085/clone_end = 3/gb = AW215489/ | P | 313.6 | P | 394.3 | P | 335.8 | P |
| | | gi = 6526165/ug = Mm.89290/len = 522/STRA = rev | | | | | | | |
| 8 | 100733_at | Cluster Incl X70303 Proteasome (prosome, macropain) subunit, alpha type 2/cds = (77,781)/gb = X70303/gi = 897783/ | P | 2239.7 | P | 1796.1 | P | 1567.9 | P |
| | | ug = Mm 3428/len = 904/STRA = for | | | | | | | |
| 8 | 93252_at | Cluster Incl X81816 M musculus BAP31 mRNA/cds = (2,739)/gb = X81816/gi = 1487985/ug = Mm 17/len = 1132/STRA = for | P | 718.8 | P | 696.3 | P | 710.9 | P |
| 8 | 93741_at | Cluster Incl AA693066: vr61b07.s1 Mus musculus cDNA, 5 end/clone = IMAGE-1125109/clone_end = 5/gb = AA693066/ | P | 263.2 | P | 234.4 | P | 222 | P |
| | | gi = 2694004/ug = Mm.21062/len = 379/STRA = for | | | | | | | |
| 8 | 94237_at | Cluster Incl AI846708.UI-M-AN1-afi-g-08-0-UI s1 Mus musculus cDNA, 3 end/clone = UI-M-AN1-afi-g-08-o-UI | P | 325.8 | P | 440.1 | P | 217.3 | A |
| | | clone_end = 3/gb = AI846708/gi = 5490614/ug = Mm 22333/len = 342/STRA = for | | | | | | | |
| 8 | 98921_at | Cluster Incl AW125612: UI-M-BH2.2-aqj-f-12-0-UI s1 Mus musculus cDNA, 3 end/clone = UI-M-BH2.2-aqj-f-12-0-UI/ | P | 307.6 | P | 401 | P | 469.3 | P |
| | | clone_end = 3/gb = AW125812/gi = 6101142/ug = Mm 28626/len = 449/STRA = for | | | | | | | |
| 8 | 98936_at | Cluster Incl AI837395: UI-M-AP0-abm-d-04-0-UI s1 Mus musculus cDNA, 3 end/clone = UI-M-AP0-abm-d-04-0-UI/ | P | 122.8 | P | 9.6 | P | 70.6 | P |
| | | clone_end = 3/gb = AI837395/gi = 5471608/ug = Mm 28688/len = 328/STRA = for | | | | | | | |
| 8 | 95052_at | Cluster Incl AI839150 UI-M-AO0-ach-e-07-0-UI s1 Mus musculus cDNA, 3 end/clone = UI-M-AO0-ach-e-07-0-UI/ | P | 327.3 | P | 357.8 | P | 190.1 | M |
| | | clone_end = 3/gb = AI839150/gi = 5473363/ug = Mm 29140/len = 462/STRA = for | | | | | | | |
| 8 | 99671_at | Cluster Incl X04673 Adipsin/cds = (19,798)/gb = X04673/gi = 49883/ug = Mm4407/len = 867/STRA = for | P | 2055.5 | P | 512.9 | P | 1527.1 | P |
| 8 | 102016_at | Cluster Incl M61737: M. musculus adipocyte-specific mRNA, partial cds/cds = UNKNOWN/gb = M61737/gi = 201107/ | P | 256.1 | P | 218.5 | P | 155.4 | P |
| | | ug = Mm 10026/len = 1725/STRA = for | | | | | | | |
| 8 | 102400_at | Cluster Incl AW125118 UI-M-BH2 1-apz-a-03-0-UI s1 Mus musculus cDNA, 3 end/clone = UI-M-BH2.1-apz-a-03-0-UI/ | P | 222.9 | P | 203 | P | 131.9 | A |
| | | clone_end = 3/gb = AW125118/gi = 6100648/ug = Mm 12441/len = 270/STRA = for | | | | | | | |
| 8 | 103398_at | Cluster Incl AW123232: UI-M-BH2 1-apd-g-08-0-UI s1 Mus musculus cDNA, 3 end/clone = UI-M-BH2 1-apd-g-08-0-UI/ | P | 257.3 | P | 280.1 | P | 162.3 | P |
| | | clone_end = 3/gb = AW123232/gi = 6098727/ug = Mm 18714/len = 469/STRA = rev | | | | | | | |
| 8 | 103646_at | Cluster Incl X85983 Carnitine acetyltransferase/cds = (125,2008)/gb = X85983/gi = 758631/ug = Mm 20396/len = 2596/ | P | 221.6 | P | 374.1 | P | 423 | P |
| | | STRA = for | | | | | | | |
| 8 | 104381_at | Cluster Incl AF085745 Mus musculus nuclear orphan receptor LXR-alpha mRNA, complete cds/cds = (84,1421)/ | P | 90.6 | P | 139.1 | P | 28.6 | P |
| | | gb = AF085745/gi = 4322470/ug = Mm 22690/len = 1665/STRA = rev | | | | | | | |
| 8 | 100900_at | Cluster Incl AW209053: un99c08 x1 Mus musculus cDNA, 3 end/clone = IMAGE-2582030/clone_end = 3/gb = AW209053/ | P | 462.8 | P | 530.9 | P | 302 | A |
| | | gi = 6514993/ug = Mm 57203/len = 446/STRA = for | | | | | | | |
| 8 | 101393_at | Cluster Incl AJ001633: Annexin III/cds = (200,1171)/gb = AJ001633/gi = 2437839/ug = Mm 7214/len = 1602/STRA = for | P | 97.6 | P | 102 | P | 129.1 | P |
| 8 | 104444_at | Cluster Incl AA689927 v163c12 r1 Mus musculus cDNA, 5 end/clone = IMAGE-1167766/clone_end = 5/gb = AA689927/ | P | 276.1 | P | 277.9 | P | 188 | A |
| | | gi = 2690863/ug = Mm 29321/len = 163/STRA = for | | | | | | | |
| 8 | 96598_at | Cluster Incl AA122714 mr02b11 r1 Mus musculus cDNA, 5 end/clone = IMAGE-5296253/clone_end = 5/gb = AA122714/ | P | 47.3 | P | 42.5 | P | 27.9 | M |
| | | gi = 1681720/ug = Mm 309/len = 506/STRA = for | | | | | | | |
| 8 | 93080_at | Cluster Incl AF069954 G protein gamma 3 linked gene/cds = (305,1537)/gb = AF069954/gi = 3885499/ug = Mm 15985/ | P | 633.2 | P | 736 | P | 487.7 | A |
| | | len = 1654/STRA = for | | | | | | | |
| 8 | 93342_at | Cluster Incl AI852665: UI-M-BH0-aji-b-02-0-UI s1 Mus musculus cDNA, 3 end/clone = UI-M-BH0-aji-b-02-0-UI/clone_end = 3/ | P | 137.6 | P | 204.8 | P | 157.5 | A |
| | | gb = AI852665/gi = 5496571/ug = Mm 18739/len = 503/STRA = for | | | | | | | |
| 8 | 94080_at | Cluster Incl AI835718: UI-M-AI0-aan-c-10-0-UI s1 Mus musculus cDNA, 3 end/clone = UI-M-AI0-aan-c-10-0-UI/ | P | 174 | P | 92.4 | A | 178.8 | P |
| | | clone_end = 3/gb = AI835718/gi = 5469931/ug = Mm 22149/len = 348/STRA = for | | | | | | | |
| 8 | 94313_at | Cluster Incl X96767: M musculus mRNA for U1 anRNP-specific protein C/cds = (31,510)/gb = X96767/gi = 1360016/ | P | 406.1 | P | 467.3 | P | 388.6 | A |
| | | ug = Mm 2265/len = 738/STRA = for | | | | | | | |
| 8 | 95427_at | Cluster Incl AI843650: UI-M-AO1-aen-h-06-0-UI s1 Mus musculus cDNA, 3 end/clone = UI-M-AO1-aen-h-06-0-UI/ | A | 502.3 | P | 463.4 | P | 238.2 | A |
| | | clone_end = 3/gb = AI843650/gi = 5477863/ug = Mm 24455/len = 449/STRA = for | | | | | | | |
| 8 | 98538_at | Cluster Incl X81632 M musculus mRNA for e1 protein/cds = UNKNOWN/gb = X81632/gi = 563507/ug = Mm 35854/len = 3769/ | P | 244.2 | P | 325.2 | P | 214.7 | M |
| | | STRA = for | | | | | | | |
| 8 | 101526_at | Cluster Incl X14759 Homeo box, msh-like 1/cds = (141,1112)/gb = X14759/gi = 51431/ug = Mm 870/len = 1810/STRA = for | P | 342.4 | P | 368.3 | A | 470.8 | P |
| 8 | 103853_at | Cluster Incl AI845664 UI-M-AQ1-ady-c-07-0-UI s1 Mus musculus cDNA, 3 end/clone = UI-M-AQ1-ady-c-07-0-UI/ | P | 79.6 | P | 102.3 | P | 50.7 | A |
| | | clone_end = 3/gb = AI845664/gi = 5489570/ug = Mm 21070/len = 184/STRA = for | | | | | | | |
| 8 | 104120_at | Cluster Incl AW049205 UI-M-BH1-anb-a-02-0-UI s1 Mus musculus cDNA, 3 end/clone = UI-M-BH1-anb-a-02-0-UI/ | P | 479.3 | P | 639.5 | P | 517.9 | A |
| | | clone_end = 3/gb = AW049205/gi = 5909734/ug = Mm 21978/len = 412/STRA = for | | | | | | | |
| 8 | 104287_at | Cluster Incl AI853807 UI-M-BH0-ajg-h-06-0-UI s1 Mus musculus cDNA, 3 end/clone = UI-M-BH0-ajg-h-06-0-UI/ | P | 480.5 | P | 533 | A | 406.2 | A |
| | | clone_end = 3/gb = AI853807/gi = 5497713/ug = Mm.22271/len = 445/STRA = for | | | | | | | |
| 8 | 97934_at | Cluster Incl U73820 Mus musculus polypeptide GalNac transferase-T1 (ppGaNTase-T1) mRNA, complete cds/ | P | 216.1 | P | 148.8 | P | 82.1 | P |
| | | cds = (0,1679)/gb = U73820/gi = 2149048/ug = Mm.30249/len = 1680/STRA = for | | | | | | | |
| 8 | 103506_f_at | Cluster Incl AW228162 up14b05 x1 Mus musculus cDNA, 3 end/clone = IMAGE-2654289/clone_end = 3/gb = AW228162/ | P | 81 | P | 103.2 | P | 72.7 | P |
| | | gi = 6557458/ug = Mm 2247/len = 414/STRA = for | | | | | | | |
| 8 | 92805_s_at | Cluster Incl Y12577 ADP-ribosylation-like 4/cds = (243,845)/gb = Y12577/gi = 3288541/ug = Mm 12723/len = 1168/STRA = for | P | 229.6 | P | 83.4 | P | −65.3 | A |
| 8 | 95453_f_at | Cluster Incl AF087687 Mus musculus calcium-binding protein S100A1 mRNA, complete cds/cds = (110,394)/gb = AF087687/ | P | 267.8 | P | 242.5 | P | 189.2 | A |
| | | gi = 3746891/ug = Mm.24662/len = 506/STRA = for | | | | | | | |
| 8 | 95480_at | Cluster Incl AI847163 UI-M-AO1-aep-d-12-0-UI s1 Mus musculus cDNA, 3 end/clone = UI-M-AO1-aep-d-12-0-UI/ | P | −76 | P | 19.4 | P | −9.1 | A |
| | | clone_end = 3/gb = AI847163/gi = 5491069/ug = Mm 24855/len = 148/STRA = for | | | | | | | |
| 8 | 96110_at | Cluster Incl U31966: Carbonyl reductase/cds = (128,961)/gb = U31966/gi = 1049107/ug = Mm.26940/len = 1053/STRA = for | P | 302.1 | P | 297.6 | A | 297.7 | A |
| 8 | 96604_at | Cluster Incl AW125827 UI-M-BH2 2-aqk-b-10-0-UI s1 Mus musculus cDNA, 3 end/clone = UI-M-BH2 2-aqk-b-10-0-UI/ | P | 175.3 | P | 152.8 | M | 150.2 | A |
| | | clone_end = 3/gb = AW125827/gi = 6101357/ug = Mm 27039/len = 461/STRA = for | | | | | | | |
| 8 | 98920_g_at | Cluster Incl AA867773: vx16d06.r1 Mus musculus cDNA, 5 end/clone = IMAGE-1284619/clone_end = 5/gb = AA867773/ | P | 199.6 | P | 228.3 | A | 299.2 | A |
| | | gi = 2963218/ug = Mm.28626/len = 385/STRA = for | | | | | | | |
| 8 | 98588_at | Cluster Incl Z11774 Fumarylacetoacetate hydrolase/cds = (31,1290)/gb = Z11774/gi = 50972/ug = Mm 3798/len = 1398/ | P | 329.6 | P | 359 | P | 278.4 | A |
| | | STRA = rev | | | | | | | |
| 8 | 102232_at | Cluster Incl AI850740 UI-M-BG1-ail-g-10-0-UI s1 Mus musculus cDNA, 3 end/clone = UI-M-BG1-ail-g-10-0-UI/ | P | 154.6 | P | 187.6 | P | 126.5 | P |
| | | clone_end = 3/gb = AI850740/gi = 5494646/ug = Mm 1040/len = 404/STRA = for | | | | | | | |
| 8 | 103735_at | Cluster Incl M89800 Wingless-related MMTV integration site 6/cds = (0,1094)/gb = M89800/gi = 202407/ug = Mm 2438/ | P | 85.5 | P | 131.2 | P | 45.1 | M |
| | | len = 1435/STRA = for | | | | | | | |
| 8 | 104695_at | Cluster Incl AI639864 vx72d06 x1 Mus musculus cDNA, 3 end/clone = IMAGE-1280747/clone_end = 3/gb = AI639864/ | P | 259.8 | P | 285.4 | P | 81.4 | A |
| | | gi = 4702973/ug = Mm.33622/len = 347/STRA = for | | | | | | | |
| 8 | 94514_s_at | Cluster Incl AI853439 UI-M-BH0-ajy-d-04-0-UI s1 Mus musculus cDNA, 3 end/clone = UI-M-BH0-ajy-d-04-0-UI/ | P | −21.9 | A | 179.8 | P | 130.6 | A |
| | | clone_end = 3/gb = AI853439/gi = 5497345/ug = Mm 28981/len = 465/STRA = for | | | | | | | |
| 8 | 99146_at | Cluster Incl AW124355 UI-M-BH2 1-apq-h-02-0-UI s1 Mus musculus cDNA, 3 end/clone = UI-M-BH2 1-apq-h-02-0-UI/ | P | 101.6 | P | 157 | P | 98.9 | A |
| | | clone_end = 3/gb = AW124355/gi = 6099885/ug = Mm 41462/len = 237/STRA = for | | | | | | | |
| 8 | 99600_at | Cluster Incl AI838337: UI-M-AO0-aby-h-03-0-UI.s1 Mus musculus cDNA, 3 end/clone = UI-M-AO0-aby-h-03-0-UI/ | A | 300.4 | P | 308.9 | A | 275 | P |
| | | clone_end = 3/gb = AI838337/gi = 5472550/ug = Mm.42855/len = 414/STRA = for | | | | | | | |
| 8 | 104132_at | Cluster Incl AW047276: UI-M-BH1-amh-c-02-0-UI.s1 Mus musculus cDNA, 3 end/clone = UI-M-BH1-amh-c-02-0-UI/ | P | 131.6 | P | 174.6 | P | 38.7 | A |
| | | clone_end = 3/gb = AW047276/gi = 5907805/ug = Mm 22072/len = 602/STRA = for | | | | | | | |
| 8 | 104318_at | Cluster Incl Z97018: Mus musculus mRNA for PEX5 protein/cds = (0,1919)/gb = Z97018/gi = 2222731/ug = Mm.22418/ | P | 126.2 | P | 124.1 | P | 122 | P |
| | | len = 1986/STRA = for | | | | | | | |
| 8 | 94956_at | Cluster Incl AF021031 DiGeorge syndrome chromosome region 6/cds = (0,584)/gb = AF021031/gi = 2582382/ug = Mm.27155/ | A | 51 | P | 85.4 | P | 41.6 | M |
| | | len = 996/STRA = for | | | | | | | |
| 8 | 99988_at | Cluster Incl AW122115 UI-M-BH2 2-aor-b-02-0-UI s1 Mus musculus cDNA, 3 end/clone = UI-M-BH2 2-aor-b-02-0-UI/ | P | 26.4 | A | 187.8 | A | 135.1 | A |
| | | clone_end = 3/gb = AW122115/gi = 6097532/ug = Mm.4471/len = 403/STRA = for | | | | | | | |
| 8 | 101728_at | Cluster Incl S46665 C5a anaphylatoxin receptor = peptidergic G-protein-coupled receptor [mice, BALB/C, Genomic, 1114 nt | P | 804 | A | 689.7 | P | 842.3 | P |
| | | 2 segments]/cds = (0,1055)/gb = S46665/gi = 1680365/ug = Mm.57044/len = 1056/STRA = for | | | | | | | |
| 8 | 94020_at | Cluster Incl Y17808 Mus musculus mRNA for A6 related protein/cds = (17,1066)/gb = Y17808/gi = 4468631/ug = Mm 21852/ | P | 150.3 | P | 154.3 | A | 178.3 | A |
| | | len = 1435/STRA = for | | | | | | | |
| 8 | 96020_at | Cluster Incl M22531: Mouse complement C1q B chain mRNA, complete cds/cds = (156,917)/gb = M22531/gi = 192266/ | P | 165.4 | P | −97.1 | A | −122.5 | A |
| | | ug = Mm.2570/len = 1059/STRA = for | | | | | | | |
| 8 | 96071_at | Cluster Incl AI835709 UI-M-AI0-aan-b-12-0-UI.s1 Mus musculus cDNA, 3 end/clone = UI-M-AI0-aan-b-12-0-UI/ | A | 1315 | A | 1561.1 | P | 1320.5 | P |
| | | clone_end = 3/gb = AI835709/gi = 5469922/ug = Mm 265/len = 395/STRA = for | | | | | | | |
| 8 | 97252_at | Cluster Incl AW120926 UI-M-BH2.3-aob-f-08-0-UI s1 Mus musculus cDNA, 3 end/clone = UI-M-BH2 3-aob-f-08-0-UI/ | P | 18.1 | P | 64 | A | −141.2 | A |
| | | clone_end = 3/gb = AW120926/gi = 6096259/ug = Mm 27871/len = 171/STRA = for | | | | | | | |
| 8 | 95738_at | Cluster Incl AW124889 UI-M-BH2.1-apu-c-11-0-UI s1 Mus musculus cDNA, 3 end/clone = UI-M-BH2 1-apu-c-11-0-UI/ | P | 103.6 | P | 73.1 | A | 118 | A |
| | | clone_end = 3/gb = AW124889/gi = 6100419/ug = Mm 29751/len = 386/STRA = for | | | | | | | |
| 8 | 98606_s_at | Cluster Incl X69656 M musculus (clone S5) WRS mRNA for tryptophan-tRNA ligase/cds = (0,1427)/gb = X69656/gi = 55435/ | P | 139.7 | P | 175.9 | P | 99.7 | A |
| | | ug = Mm 38433/len = 1428/STRA = for | | | | | | | |
| 8 | 101027_s_at | Cluster Incl AF069051 Mus musculus pituitary tumor transfroming gene protein (PTTG) mRNA, complete cds/ | P | 552.9 | P | 382.9 | A | 314.9 | A |
| | | cds = (303,893)/gb = AF069051/gi = 3702843/ug = Mm 6856/len = 945/STRA = for | | | | | | | |
| 8 | 103643_at | Cluster Incl AI844784 UI-M-AL1-ahq-g-12-0-UI s1 Mus musculus cDNA, 3 end/clone = UI-M-AL1-ahq-g-12-0-UI/ | P | 163.5 | P | 210.8 | A | 153.6 | M |
| | | clone_end = 3/gb = AI844784/gi = 5488690/ug = Mm 20387/len = 264/STRA = rev | | | | | | | |
| 8 | 104354_at | Cluster Incl X06368 Colony stimulating factor 1 receptor/cds = (75,3005)/gb = X06368/gi = 50980/ug = Mm.22574/len = 3665/ | P | 372.3 | P | 167.6 | A | 83 | A |
| | | STRA = for | | | | | | | |
| 8 | 104719_at | Cluster Incl AI182203 uc68a04 r1 Mus musculus cDNA, 5 end/clone = IMAGE-1430766/clone_end = 5/gb = AI182203/ | P | 139.5 | P | 153.5 | A | 22.5 | A |
| | | gi = 3732841/ug = Mm 24510/len = 517/STRA = for | | | | | | | |
| 8 | 92182_at | Cluster Incl AI836877 UI-M-AP0-abg-c-08-0-UI s2 Mus musculus cDNA, 3 end/clone = UI-M-AP0-abg-c-08-0-UI/ | P | 138.8 | P | 123.8 | M | 43.6 | A |
| | | clone_end = 3/gb = AI836877/gi = 5471090/ug = Mm 33851/len = 395/STRA = for | | | | | | | |
| 8 | 93856_at | Cluster Incl M55512 Wilms tumor homolog/cds = UNKNOWN/gb = M55512/gi = 202413/ug = Mm 87561/len = 3089/ | A | 175.4 | P | 167.5 | A | 134.4 | A |
| | | STRA = for | | | | | | | |
| 8 | 94512_f_at | Cluster Incl AI843210 UI-M-AK1-aet-f-05-0-UI s1 Mus musculus cDNA, 3 end/clone = UI-M-AK1-aet-f-05-0-UI/ | P | 86.3 | P | 121.5 | P | 126.2 | A |
| | | clone_end = 3/gb = AI843210/gi = 5477423/ug = Mm 28981/len = 418/STRA = for | | | | | | | |
| 8 | 94515_at | Cluster Incl AW208628 uo62a08 x1 Mus musculus cDNA, 3 end/clone = IMAGE-2647094/clone_end = 3/gb = AW208628/ | P | 90.6 | P | 14.3 | A | 61 | A |
| | | gi = 6514568/ug = Mm.28986/len = 601/STRA = for | | | | | | | |
| 8 | 94561_at | Clustar Incl AI836140 UI-M-AQ0-aai-b-09-0-UI s2 Mus musculus cDNA, 3 end/clone = UI-M-AQ0-aai-b-09-0-UI/ | P | 480.6 | P | 450.1 | A | 505.8 | A |
| | | clone_end = 3/gb = AI836140/gi = 5470353/ug = Mm 29107/len = 498/STRA = for | | | | | | | |
| 8 | 97531_at | Cluster Incl X95280 Mus musculus mRNA for GOS2-like protein/cds = (132,443)/gb = X95280/gi = 1213012/ug = Mm 3283/ | A | 294.8 | P | 211.2 | P | 173.9 | A |
| | | len = 752/STRA = for | | | | | | | |
| 8 | 101062_at | Cluster Incl AF054670: Heme oxygenase (decycling) 2/cds = (98,1045)/gb = AF054670/gi = 2984775/ug = Mm 710/len = 1192/ | P | 50.6 | P | 41.9 | A | 2.3 | A |
| | | STRA = for | | | | | | | |
| 8 | 102366_at | Cluster Incl AA718169 yu54h05 r1 Mus musculus cDNA, 5 end/clone = IMAGE-1195257/clone_end = 5/gb = AA718169/ | P | 177 | M | 455.3 | A | 301 | A |
| | | gi = 2730443/ug = Mm 1181/len = 290/STRA = for | | | | | | | |
| 8 | 101842_g_at | Cluster Incl AW049716 UI-M-BH1-ani-e-11-0-UI s1 Mus musculus cDNA, 3 end/clone = UI-M-BH1-ani-e-11-0-UI/ | A | 134.2 | A | 196.8 | A | 79.7 | A |
| | | clone_end = 3/gb = AW049716/gi = 5910245/ug = Mm 8534/len = 524/STRA = for | | | | | | | |
| 8 | 93613_at | Cluster Incl AB008192 Mus musculus mRNA for mSmad3, complete cds/cds = (26,1303)/gb = AB008192/gi = 4630836/ | A | 34.4 | A | 53.8 | A | 43.6 | P |
| | | ug = Mm 7320/len = 1428/STRA = for | | | | | | | |
| 9 | 92611_at | Cluster Incl U18773 Mus musculus GPI-anchored protein p137 mRNA, partial cds/cds = (0,550)/gb = U18773/gi = 862634/ | P | 1278.1 | P | 1266.2 | P | 997.5 | P |
| | | ug = Mm.1098/len = 2707/STRA = for | | | | | | | |
| 9 | 92623_at | Cluster Incl AI847171: UI-M-AO1-aep-e-08-0-UI s1 Mus musculus cDNA, 3 end/clone = UI-M-AO1-aep-e-08-0-UI/ | P | 1690.7 | P | 1718.2 | P | 1564 | P |
| | | clone_end = 3/gb = AI847171/gi = 5491077/ug = Mm 11204/len = 208/STRA = for | | | | | | | |
| 9 | 92639_at | Cluster Incl U80932 Senne/threonine kinase 6/cds = (48,1235)/gb = U80932/gi = 1763646/ug = Mm.11738/len = 1643/ | P | 191.1 | P | 179.6 | P | 117 | P |
| | | STRA = for | | | | | | | |
| 9 | 92644_s_at | Cluster Incl M12848 Mouse myb proto-oncogene mRNA encoding 71 kd myb protein, complete cds/cds = (161,2071)/ | P | 175.7 | P | 112.9 | P | 72.8 | P |
| | | gb = M12848/gi = 199934/ug = Mm 1202/len = 3310/STRA = for | | | | | | | |
| 9 | 92647_at | Cluster Incl U35141: Mus musculus retinoblastoma-binding protein (mRbAp48) mRNA, complete cds/cds = (107,1492)/ | P | 933.2 | P | 1165.6 | P | 952.1 | P |
| | | gb = U35141/gi = 1016274/ug = Mm.12145/len = 1961/STRA = for | | | | | | | |
| 9 | 92790_at | Cluster Incl D55720 Karyopherin (importin) alpha 2/cds = (177,1766)/gb = D55720/gi = 893392/ug = Mm 12508/len = 2048/ | P | 699.3 | P | 601.9 | P | 251.4 | P |
| | | STRA = for | | | | | | | |
| 9 | 92809_r_at | Cluster Incl X17069 Mouse mRNA for protein with homology to transition protein 2 (TP2)/cds = (123,1358)/gb = X17069/ | P | 1081.4 | P | 1766.8 | P | 1139.3 | P |
| | | gi = 54875/ug = Mm 12758/len = 2026/STRA = for | | | | | | | |
| 9 | 92826_at | Cluster Incl Y17852 Mus musculus mRNA for ganglioside-induced differentiation associated protein 3/cds = (0,385)/ | P | 396 | P | 370.2 | P | 265.6 | P |
| | | gb = Y17852/gi = 3378457/ug = Mm 12962/len = 651/STRA = for | | | | | | | |
| 9 | 92829_at | Cluster Incl U09659 Mus musculus chaperonin 10 mRNA, complete cds/cds = (78,386)/gb = U09659/gi = 495205/ | P | 1565.8 | P | 1525 | P | 1246 | P |
| | | ug = Mm.12970/len = 462/STRA = for | | | | | | | |
| 9 | 93013_at | Cluster Incl AF077861 Inhibitor of DNA binding 2/cds = (0,404)/gb = AF077861/gi = 3420740/ug = Mm.1466/len = 405/ | P | 590.3 | P | 554.3 | P | 510.1 | P |
| | | STRA = for | | | | | | | |
| 9 | 93041_at | Cluster Incl D26089: Mini chromosome maintenance deficient 4 homolog (S. cerevisiae)/cds = (126,2714)/gb = D26089/ | P | 549.8 | P | 416.8 | P | 205.8 | P |
| | | gi = 940405/ug = Mm.1500/len = 3221/STRA = for | | | | | | | |
| 9 | 93051_at | Cluster Incl Z37107: M. musculus mRNA for epoxide hydrolase/cds = (17,1681)/gb = Z37107/gi = 563509/ug = Mm.15295/ | P | 167.5 | P | 171.9 | P | 148.9 | P |
| | | len = 1990/STRA = for | | | | | | | |
| 9 | 93069_at | Cluster Incl U62483 Mus musculus ubiquitin conjugating enzyme (ubc4) mRNA, complete cds/cds = (386,829)/gb = U62483/ | P | 235.6 | P | 184.2 | P | 143.9 | P |
| | | gi = 1480741/ug = Mm 1570/len = 2410/STRA = for | | | | | | | |
| 9 | 93095_at | Cluster Incl U00431: Mus musculus HMG-1 mRNA, complete cds/cds = (145,792)/gb = U00431/gi = 437101/ug = Mm 16421/ | P | 572.9 | P | 514.2 | P | 346.2 | P |
| | | len = 1308/STRA = for | | | | | | | |
| 9 | 93117_at | Cluster Incl AF073993: Mus musculus heterogenous nuclear ribonucleoprotein A2/B1 (hnRNP A2/B1) mRNA, complete cds/ | P | 3263.8 | P | 3257.9 | P | 1475.2 | P |
| | | cds = (96,1121)/gb = AF073993/gi = 3329497/ug = Mm 16767/len = 1677/STRA = for | | | | | | | |
| 9 | 93250_r_at | Cluster Incl X67668 High mobility group protein 2/cds = (135,752)/gb = X67668/gi = 51338/ug = Mm 1693/len = 847/ | P | 1316.5 | P | 1208.4 | P | 652.4 | P |
| | | STRA = for | | | | | | | |
| 9 | 93512_f_at | Cluster Incl AW121801 UI-M-BH2 3-anx-b-02-0-UI s2 Mus musculus cDNA, 3 end/clone = UI-M-BH2 3-anx-b-02-0-UI/ | P | 682.2 | P | 500.5 | P | 372.2 | P |
| | | clone_end = 3/gb = AW121801/gi = 6097161/ug = Mm 19352/len = 406/STRA = for | | | | | | | |
| 9 | 93519_s_at | Cluster Incl AI847056 UI-M-AP1-agn-a-10-0-UI s1 Mus musculus cDNA, 3 end/clone = UI-M-AP1-agn-a-10-0-UI/ | P | 1251.7 | P | 1126.8 | P | 1025.6 | P |
| | | clone_end = 3/gb = AI847056/gi = 5490962/ug = Mm 196/len = 185/STRA = for | | | | | | | |
| 9 | 93541_at | Cluster Incl Z68618 Transgelin/cds = (12,617)/gb = Z68618/gi = 1160197/ug = Mm 2006/len = 1016/STRA = for | P | 687.9 | P | 853.3 | P | 727.6 | P |
| 9 | 93562_at | Cluster Incl AI854366 UI-M-BG1-aic-e-10-0-UI s1 Mus musculus cDNA, 3 end/clone = UI-M-BG1-aic-e-10-0-UI/ | P | 1606.2 | P | 1102.9 | P | 950.9 | P |
| | | clone_end = 3/gb = AI854366/gi = 5498272/ug = Mm 2033/len = 410/STRA = for | | | | | | | |
| 9 | 93772_i_at | Cluster Incl AI835041 UI-M-AI1-afn-d-02-0-UI s1 Mus musculus cDNA, 3 end/clone = UI-M-AI1-afn-d-02-0-UI/clone_end = 3/ | P | 1060.5 | P | 735.7 | P | 542.7 | P |
| | | gb = AI835041/gi = 5469254/ug = Mm 21203/len = 409/STRA = for | | | | | | | |
| 9 | 93786_i_at | Cluster Incl AI837302 UI-M-AK0-ade-h-11-0-UI s1 Mus musculus cDNA, 3 end/clone = UI-M-AK0-ade-h-11-0-UI | P | 728.4 | P | 692.9 | P | 390.5 | P |
| | | clone_end = 3/gb = AI837302/gi = 5471515/ug = Mm 21356/len = 392/STRA = for | | | | | | | |
| 9 | 93787_f_at | Cluster Incl AI837302: UI-M-AK0-ade-h-11-0-UI s1 Mus musculus cDNA, 3 end/clone = UI-M-AK0-ade-h-11-0-UI/ | P | 1747.6 | P | 1623.7 | P | 1438.4 | P |
| | | clone_end = 3/gb = AI837302/gi = 5471515/ug-Mm 21356/len = 392/STRA = for | | | | | | | |
| 9 | 93805_at | Cluster Incl AW121164 UI-M-BH2.3-aoh-g-12-0-UI s1 Mus musculus cDNA, 3 end/clone = UI-M-BH2 3-aoh-g-12-0-UI/ | P | 680.3 | P | 594.3 | P | 434.3 | P |
| | | clone_end = 3/gb = AW121164/gi = 6096497/ug = Mm 21444/len = 423/STRA = for | | | | | | | |
| 9 | 93820_at | Cluster Incl AF037371 Mus musculus cytochrome c oxidase subunit VIIa-L precursor (Cox7al) mRNA, nuclear gene | P | 1806.5 | P | 2208.2 | P | 1617.5 | P |
| | | encoding mitochondrial protein, complete cds/cds = (70,321)/gb = AF037371/gi = 2708817/ug = Mm.2151/len = 461/ | | | | | | | |
| | | STRA = for | | | | | | | |
| 9 | 93997_at | Cluster Incl AI853475 UI-M-BH0-ajy-h-08-0-UI s1 Mus musculus cDNA, 3 end/clone = UI-M-BH0-ajy-h-08-0-UI/ | P | 482 | P | 431.9 | P | 442.4 | P |
| | | clone_end = 3/gb = AI853475/gi = 5497381/ug-Mm 21761/len = 472/STRA = for | | | | | | | |
| 9 | 93999_at | Cluster Incl AW060597 UI-M-BH1-anq-d-09-0-UI s1 Mus musculus cDNA, 3 end/clone = UI-M-BH1-anq-d-09-0-UI/ | P | 1568.4 | P | 1774.4 | P | 1146.1 | P |
| | | clone_end = 3/gb = AW060597/gi = 6008348/ug = Mm 21764/len = 224/STRA = for | | | | | | | |
| 9 | 94030_at | Cluster Incl AI853431: UI-M-BH0-ajy-c-06-0-UI s1 Mus musculus cDNA, 3 end/clone = UI-M-BH0-ajy-c-06-0-UI/ | P | 261.1 | P | 274.7 | P | 237.2 | P |
| | | clone_end = 3/gb = AI853431/gi = 5497337/ug = Mm 21901/len = 250/STRA = for | | | | | | | |
| 9 | 94073_at | Cluster Incl AI836182 UI-M-AQ0-aaj-h-11-0-UI s2 Mus musculus cDNA, 3 end/clone = UI-M-AQ0-aaj-h-11-0-UI/ | P | 795.6 | P | 918.2 | P | 397.2 | P |
| | | clone_end = 3/gb = AI836182/gi = 5470395/ug = Mm.22117/len = 542/STRA = for | | | | | | | |
| 9 | 94228_at | Cluster Incl AW123788 UI-M-BH2.1-apm-d-07-0-UI s1 Mus musculus cDNA, 3 end/clone = UI-M-BH2 1-apm-d-07-0-UI/ | P | 478.5 | P | 469.1 | P | 297.8 | P |
| | | clone_end = 3/gb = AW123788/gi = 6099318/ug-Mm 22269/len = 174/STRA = rev | | | | | | | |
| 9 | 94267_i_at | Cluster Incl AW124386 UI-M-BH2 1-ape-b-11-0-UI s1 Mus musculus cDNA, 3 end/clone = UI-M-BH2 1-ape-d-11-0-UI/ | P | 1674.2 | P | 1245.2 | P | 861.5 | P |
| | | clone_end = 3/gb = AW124386/gi = 6099916/ug = Mm.22470/len = 121/STRA = for | | | | | | | |
| 9 | 94268_f_at | Cluster Incl AW124386 UI-M-BH2 1-ape-b-11-0-UI s1 Mus musculus cDNA, 3 end/clone = UI-M-BH2 1-ape-b-11-0-UI/ | P | 2048.9 | P | 2196.9 | P | 1650.8 | P |
| | | clone_end = 3/gb = AW124386/gi = 6099916/ug = Mm 22470/len = 121/STRA = for | | | | | | | |
| 9 | 94294_at | Cluster Incl X66032 M musculus mRNA for cyclin B2/cds = (101,1297)/gb = X66032/gi = 50612/ug = Mm 22592/len = 1528/ | P | 1108.8 | P | 1429.7 | P | 657.2 | P |
| | | STRA = for | | | | | | | |
| 9 | 94806_at | Cluster Incl AW125336 UI-M-BH2 1-apy-g-01-0-UI s1 Mus musculus cDNA, 3 end/clone = UI-M-BH2.1-apy-g-01-0-UI/ | P | 1055.6 | P | 909.2 | P | 587 | P |
| | | clone_end = 3/gb = AW125336/gi = 6100866/ug = Mm 22676/len = 253/STRA = for | | | | | | | |
| 9 | 94885_at | Cluster Incl AI840062 UI-M-AL0-abt-b-05-0-UI s1 Mus musculus cDNA, 3 end/clone = UI-M-AL0-abt-b-05-0-UI/ | P | 227.1 | P | 214.7 | P | 103 | P |
| | | clone_end = 3/gb = AI840062/gi = 5474275/ug = Mm 23889/len = 499/STRA = for | | | | | | | |
| 9 | 95423_at | Cluster Incl Y00884 Mouse mRNA (3 4J) from concanavalin A-stimulated LB3 T-cells/cds = (22,1770)/gb = Y00884/ | P | 454.5 | P | 510.9 | P | 287.3 | P |
| | | gi = 49727/ug = Mm 2442/len = 1957/STRA = for | | | | | | | |
| 9 | 95451_at | Cluster Incl AA855382: vw72a04.r1 Mus musculus cDNA, 5 end/clone = IMAGE-1260462/clone_end = 5/gb = AA855382/ | P | 367 | P | 434.7 | P | 328.3 | P |
| | | gi = 2942920/ug = Mm 24641/len = 314/STRA = for | | | | | | | |
| 9 | 95479_at | Cluster Incl X95591 M. musculus mRNA for C1D protein/cds = (77,502)/gb = X95591/gi = 1185124/ug = Mm 2485/len = 1050/ | P | 273.3 | P | 223.4 | P | 172.4 | P |
| | | STRA = for | | | | | | | |
| 9 | 96054_f_at | Cluster Incl Y17345 Mus musculus mRNA for protein-tyrosine-phosphatase IF1/cds = (0,476)/gb = Y17345/gi = 3294228/ | P | 423.9 | P | 427.5 | P | 278.7 | P |
| | | ug = Mm 26166/len = 477/STRA = for | | | | | | | |
| 9 | 96658_at | Cluster Incl AI841906: UI-M-AO0-acd-e-10-0-UI s1 Mus musculus cDNA, 3 end/clone = UI-M-AO0-acd-e-10-0-UI/ | P | 473.1 | P | 550.9 | P | 370 | P |
| | | clone_end = 3/gb = AI841906/gi = 5476119/ug = Mm 27344/len = 417/STRA = for | | | | | | | |
| 9 | 96676_at | Cluster Incl AW121875 UI-M-BH2.3-aoi-d-05-0-UI s1 Mus musculus cDNA, 3 end/clone = UI-M-BH2 3-aoi-d-05-0-UI/ | P | 546.7 | P | 611 | P | 364.1 | P |
| | | clone_end = 3/gb = AW121875/gi = 6097249/ug = Mm.27406/len = 438/STRA = for | | | | | | | |
| 9 | 96693_at | Cluster Incl AI849453 UI-M-AJ1-ahc-h-03-0-UI s1 Mus musculus cDNA, 3 end/clone = UI-M-AJ1-ahc-h-03-0-UI/ | P | 616 | P | 708.6 | P | 499.3 | P |
| | | clone_end = 3/gb = AI849453/gi = 5493359/ug = Mm.27526/len = 291/STRA = for | | | | | | | |
| 9 | 96707_at | Cluster Incl D10630: Zinc finger protein 38/cds = (105,1772)/gb = D10630/ug = Mm 2760/len = 2103/STRA = for | P | 318.9 | P | 252.4 | P | 311.2 | P |
| 9 | 96711_at | Cluster Incl AI849191 UI-M-AJ1-agy-g-09-0-UI s1 Mus musculus cDNA, 3 end/clone = UI-M-AJ1-agy-g-09-0-UI/ | P | 388.2 | P | 355.8 | P | 306.6 | P |
| | | clone_end = 3/gb = AI849191/gi = 5493097/ug = Mm.27635/len = 469/STRA = for | | | | | | | |
| 9 | 97200_f_at | Cluster Incl X65704 M musculus mRNA for small nuclear ribonucleoprotein E/cds = (0,245)/gb = X65704/gi = 312004/ | P | 2967 | P | 3239.1 | P | 2474.9 | P |
| | | ug = Mm.27669/len = 387/STRA = for | | | | | | | |
| 9 | 97201_s_at | Cluster Incl AA823381: vp39a01.r1 Mus musculus cDNA, 5 end/clone = IMAGE-1079016/clone_end = 5/gb = AA823381/ | P | 1484.2 | P | 1155.5 | P | 839.2 | P |
| | | gi = 2893249/ug = Mm 27677/len = 339/STRA = for | | | | | | | |
| 9 | 97254_at | Cluster Incl AA690061: vr7Bd03.s1 Mus musculus cDNA, 5 end/clone = IMAGE-1134821/clone_end = 5/gb = AA690061/ | P | 609.5 | P | 492.3 | P | 332.1 | P |
| | | gi = 2690997/ug = Mm.27875/len = 331/STRA = for | | | | | | | |
| 9 | 97296_at | Cluster Incl AW124918: UI-M-BH2.1-apu-f-07-0-UI.s1 Mus musculus cDNA, 3 end/clone = UI-M-BH2.1-apu-f-07-0-UI/ | P | 222.1 | P | 253.1 | P | 146.9 | P |
| | | clone _end = 3/gb = AW124918/gi = 6100448/ug = Mm.28039/len = 187/STRA = for | | | | | | | |
| 9 | 97301_at | Cluster Incl AW124187: UI-M-BH2.1-aph-a-06-0-UI.s1 Mus musculus cDNA, 3 end/clone = UI-M-BH2.1-aph-a-06-0-UI/ | P | 1550.3 | P | 1638.4 | P | 1382.1 | P |
| | | clone_end = 3/gb = AW124187/gb = 6099717/ug = Mm.28045/len = 382/STRA = for | | | | | | | |
| 9 | 97307_f_at | Cluster Incl AI842808: UI-M-AP1-agi-f-09-0-UI.s1 Mus musculus cDNA, 3 end/clone = UI-M-AP1-agi-f-09-0-UI/clone_end = 3/ | P | 3210.9 | P | 3463.1 | P | 3207.9 | P |
| | | gb = AI842808/gi = 5477021/ug = Mm.28058/len = 337/STRA = for | | | | | | | |
| 9 | 97315_at | Cluster Incl AW121730: UI-M-BH2.3-aoe-b-02-0-UI.s1 Mus musculus cDNA, 3 end/clone = UI-M-BH2.3-aoe-b-02-0-UI/ | P | 449.2 | P | 413.7 | P | 271 | P |
| | | clone _end = 3/gb = AW121730/gi = 6097063/ug = Mm.28097/len = 372/STRA = for | | | | | | | |
| 9 | 97317_at | Cluster Incl AW122933: UI-M-BH2.1-apa-h-09-0-UI.s1 Mus musculus cDNA, 3 end/clone = UI-M-BH2.1-apa-h-09-0-UI/ | P | 877.3 | P | 756.1 | P | 452.6 | P |
| | | clone_end = 3/gb = AW122933/gi = 6098463/ug = Mm.28107/len = 399/STRA = for | | | | | | | |
| 9 | 97846_at | Cluster Incl AI848968: UI-M-AJ1-ahd-c-01-0-UI.s1 Mus musculus cDNA, 3 end/clone = UI-M-AJ1-ahd-c-01-0-UI/ | P | 489.2 | P | 442.3 | P | 445.4 | P |
| | | clone_end = 3/gb = AI848968/gb = 5492874/ug = Mm.28270/len = 391/STRA = for | | | | | | | |
| 9 | 97900_at | Cluster Incl AI845714: UI-M-AQ1-ady-h-04-0-UI.s1 Mus musculus cDNA, 3 end/clone = UI-M-AQ1-ady-h-04-0-UI/ | P | 528 | P | 513.5 | P | 537.1 | P |
| | | clone_end = 3/gb = AI845714/gi = 5489620/ug = Mm.28438/len = 384/STRA = for | | | | | | | |
| 9 | 98914_at | Cluster Incl AI853634: UI-M-BH0-ajp-h-08-0-UI.s1 Mus musculus cDNA, 3 end/clone = UI-M-BH0-ajp-h-08-0-UI/ | P | 824.8 | P | 605.8 | P | 466.3 | P |
| | | clone_end = 3/gb = AI853634/gi = 5497540/ug = Mm.28606/len = 374/STRA = for | | | | | | | |
| 9 | 98915_at | Cluster Incl AI849082: UI-M-AH1-agw-e-11-0-UI.s1 Mus musculus cDNA, 3 end/clone = UI-M-AH1-agw-e-11-0-UI/ | P | 556.7 | P | 599.8 | P | 486.7 | P |
| | | clone_end = 3/gb = AI849082/gi = 5492988/ug = Mm.28614/len = 494/STRA = for | | | | | | | |
| 9 | 94489_at | Cluster Incl U84411: Mus musculus protein tyrosine phosphatase (PRL-1) mRNA, complete cds/cds = (713,1234)/ | P | 2128.9 | P | 2179.6 | P | 1589.8 | P |
| | | gb = U84411/gi = 1814023/ug = Mm.28909/len = 2849/STRA = for | | | | | | | |
| 9 | 94508_at | Cluster Incl AI853772: UI-M-BH0-ajg-e-02-0-UI.s1 Mus musculus cDNA, 3 end/clone = UI-M-BH0-ajg-e-02-0-UI/ | P | 209.7 | P | 240.2 | P | 197.5 | P |
| | | clone_end = 3/gb = AI853772/gi = 5497678/ug = Mm.28963/len = 394/STRA = for | | | | | | | |
| 9 | 95045_at | Cluster Incl AI844469: UI-M-AJ1-ahe-d-03-0-UI.s1 Mus musculus cDNA, 3 end/clone = UI-M-AJ1-ahe-d-03-0-UI/ | P | 566.2 | P | 439.8 | P | 228.4 | P |
| | | clone_end = 3/gb = AI844489/gi = 5488375/ug = Mm.29122/len = 406/STRA = for | | | | | | | |
| 9 | 95063_at | Cluster Incl AI606257: vo03b06 x1 Mus musculus cDNA, 3 end/clone = IMAGE-1040339/clone_end = 3/gb = AI606257/ | P | 294.1 | P | 257.6 | P | 199.9 | P |
| | | gi = 4615424/ug = Mm.29164/len = 366/STRA = for | | | | | | | |
| 9 | 95069_at | Cluster Incl AA940430: vz48h05 r1 Mus musculus cDNA, 5 end/clone = IMAGE-1329753/clone_end = 5/gb = AA940430/ | P | 766.9 | P | 683.3 | P | 351.7 | P |
| | | gi = 3099974/ug = Mm.29187/len = 468/STRA = for | | | | | | | |
| 9 | 95105_at | Cluster Incl AI847697: UI-M-AP1-agm-d-08-0-UI.s1 Mus musculus cDNA, 3 end/clone = UI-M-AP1-agm-d-08-0-UI/ | P | 1286.9 | P | 1270.1 | P | 1102.1 | P |
| | | clone_end = 3/gb = AI847697/gi = 5491603/ug = Mm.29353/len = 441/STRA = for | | | | | | | |
| 9 | 95109_at | Cluster Incl AW121447: UI-M-BH2.2-aon-d-07-0-UI.s1 Mus musculus cDNA, 3 end/clone = UI-M-BH2.2-aon-d-07-0-UI/ | P | 446.5 | P | 431.2 | P | 206.3 | P |
| | | clone_end = 3/gb = AW121447/gi = 6096780/ug = Mm.29363/len = 278/STRA = for | | | | | | | |
| 9 | 95119_at | Cluster Incl AA866888: vx90e09 r1 Mus musculus cDNA, 5 end/clone = IMAGE-1282504/clone_end = 5/gb = AA866888/ | P | 280.8 | P | 191.5 | P | 153.7 | P |
| | | gi = 2962333/ug = Mm 29382/len = 420/STRA = for | | | | | | | |
| 9 | 95673_s_at | Cluster Incl AW124113: UI-M-BH2.1-apr-b-09-0-UI.s1 Mus musculus cDNA, 3 end/clone = UI-M-BH2.1-apr-b-09-0-UI/ | P | 2407 | P | 2561 | P | 2022.8 | P |
| | | clone_end = 3/gb = AW124113/gi = 6099643/ug = Mm.29588/len = 265/STRA = for | | | | | | | |
| 9 | 95699_f_at | Cluster Incl AI848094: UI-M-AP1-agk-d-05-0-UI.s1 Mus musculus cDNA, 3 end/clone = UI-M-AP1-agk-d-05-0-UI/ | P | 1222.1 | P | 1052.8 | P | 1016.9 | P |
| | | clone_end = 3/gb = AI848094/gi = 5492000/ug = Mm.29685/len = 399/STRA = for | | | | | | | |
| 9 | 96865_at | Cluster Incl M60474: Mynstoylated alanine rich protein kinase C substrate/cds = (380,1309)/gb = M60474/gi = 199026/ | P | 1483.3 | P | 975.6 | P | 960.3 | P |
| | | ug = Mm.30059/len = 2597/STRA = for | | | | | | | |
| 9 | 96891_at | Cluster Incl AI842771: UI-M-AP1-agi-c-05-0-UI.s1 Mus musculus cDNA, 3 end/clone = UI-M-AP1-agi-c-05-0-UI/ | P | 1990.3 | P | 2569.2 | P | 1659.8 | P |
| | | clone_end = 3/gb = AI842771/gi = 5476984/ug = Mm.30093/len = 354/STRA = for | | | | | | | |
| 9 | 96899_at | Cluster Incl AW123802: UI-M-BH2.1-apm-e-09-0-UI.s1 Mus musculus cDNA, 3 end/clone = UI-M-BH2.1-apm-e-09-0-UI/ | P | 1478.8 | P | 1307.2 | P | 1000.1 | P |
| | | clone_end = 3/gb = AW123802/gi = 6099332/ug = Mm.30113/len = 371/STRA = for | | | | | | | |
| 9 | 96947_at | Cluster Incl AW046273: UI-M-BH1-ala-f-05-0-UI.s1 Mus musculus cDNA, 3 end/clone = UI-M-BH1-ala-f-05-0-UI/ | P | 1450.6 | P | 1495.8 | P | 1137.1 | P |
| | | clone_end = 3/gb = AW046273/gi = 5906802/ug = Mm.30200/len = 374/STRA = for | | | | | | | |
| 9 | 96951_at | Cluster Incl AI839795: UI-M-AP0-abf-a-02-0-UI.s2 Mus musculus cDNA, 3 end/clone = UI-M-AP0-abf-a-02-0-UI/ | P | 1001.4 | P | 937.5 | P | 600.5 | P |
| | | clone_end = 3/gb = AI839795/gi = 5474048/ug = Mm.30206/len = 475/STRA = for | | | | | | | |
| 9 | 97445_at | Cluster Incl AW122022: UI-M-BH2.2-aoo-a-09-0-UI.s1 Mus musculus cDNA, 3 end/clone = UI-M-BH2.2-aoo-a-09-0-UI/ | P | 416.9 | P | 410.4 | P | 180 | P |
| | | clone_end = 3/gb = AW122022/gi = 6097428/ug = Mm.30242/len = 507/STRA = for | | | | | | | |
| 9 | 97468_at | Cluster Incl AB025409: Mus musculus mRNA for aid1334p, complete cds/cds = (78,317)/gb = AB025409/gi = 5931566/ | P | 762.6 | P | 566.4 | P | 379.7 | P |
| | | ug = Mm.3049/len = 757/STRA = for | | | | | | | |
| 9 | 97474_f_at | Cluster Incl D90225: Pleiotrophin/cds = (214,720)/gb = D90225/gi = 220553/ug = Mm.3063/len = 1514/STRA = for | P | 191 | P | 196.3 | P | 90.8 | P |
| 9 | 97478_at | Cluster Incl AW046965: UI-M-BH1-alp-c-09-0-UI.s1 Mus musculus cDNA, 3 end/clone = UI-M-BH1-alp-c-09-0-UI/ | P | 355 | P | 292.4 | P | 254 | P |
| | | clone_end = 3/gb = Aw046965/gi = 5907494/ug = Mm.30718/len = 368/STRA = for | | | | | | | |
| 9 | 97527_at | Cluster Incl AA681998: vr45a06 s1 Mus musculus cDNA, 5 end/clone = IMAGE-1123570/clone_end = 5/gb = AA681998/ | P | 699 | P | 570.5 | P | 202.8 | P |
| | | gi = 2664138/ug = Mm.326/len = 463/STRA = for | | | | | | | |
| 9 | 97543_at | Cluster Incl D49382: Mus musculus Nedd5 mRNA for septin, complete cds/cds = (53,1138)/gb = D49382/gi = 4519167/ | P | 1491.8 | P | 982.6 | P | 659.7 | P |
| | | ug = Mm.336/len = 3170/STRA = for | | | | | | | |
| 9 | 97556_at | Cluster Incl AI843178: UI-M-AK1-aet-c-07-0-UI.s1 Mus musculus cDNA, 3 end/clone = UI-M-AK1-aet-c-07-0-UI/ | P | 726 | P | 595.7 | P | 436.2 | P |
| | | clone_end = 3/gb = AI843178/gi = 5477391/ug = Mm.33875/len = 452/STRA = for | | | | | | | |
| 9 | 98038_at | Cluster Incl AF022465: Mus musculus high mobility group protein homolog HMG4 (Hmg4) mRNA, complete cds/ | P | 1300.6 | P | 1263.5 | P | 1056.9 | P |
| | | cds = (27,829)/gb = AF022465/gi = 2738988/ug = Mm.340/len = 1502/STRA = for | | | | | | | |
| 9 | 98072_f_at | Cluster Incl X77731: M. musculus mRNA for Deoxycytidine kinase/cds = (12,794)/gb = X77731/gi = 456676/ug = Mm.3446 | P | 112.6 | P | 91.4 | P | 45.5 | P |
| | | len = 2812/STRA = for | | | | | | | |
| 9 | 98075_at | Cluster Incl AW121683: UI-M-BH2.3-aoc-e-07-0-UI.s1 Mus musculus cDNA, 3 end/clone = UI-M-BH2.3-aoc-e-07-0-UI/ | P | 446.8 | P | 327.1 | P | 296.4 | P |
| | | clone_end = 3/gb = AW121683/gi = 6097916/ug = Mm.34526/len = 543/STRA = for | | | | | | | |
| 9 | 98128_at | Cluster Incl U77128: Mus musculus mitochondrial ATP synthase coupling factor 6 mRNA, nuclear gene encoding | P | 1264.6 | P | 1260.8 | P | 1091 | P |
| | | mitochondrial protein, complete cds/cds = (7,333)/gb = U77128/gi = 1679742/ug = Mm.353/len = 425/STRA = for | | | | | | | |
| 9 | 98550_at | Cluster Incl AI854006: UI-M-BH0-alv-h-10-0-UI.s1 Mus musculus cDNA, 3 end/clone = UI-M-BH0-alv-h-10-0-UI/ | P | 1577.3 | P | 1921.5 | P | 1231.4 | P |
| | | clone_end = 3/gb = AI854006/gi = 5497912/ug = Mm.36677/len = 407/STRA = for | | | | | | | |
| 9 | 98573_r_at | Cluster Incl X58045: RAN binding protein 1/cds = (137,748)/gb = X56045/gi = 443997/ug = Mm 3752/len = 1044/STRA = for | P | 1112.7 | P | 1092 | P | 954.5 | P |
| 9 | 98610_at | Cluster Incl AW047926 UI-M- BH1-aln-c-12-0-UI s1 Mus musculus cDNA, 3 end/clone = UI-M-BH1-aln-c-12-0-UI/ | P | 226 | P | 182.5 | P | 197.4 | P |
| | | clone_end = 3/gb = AW047926/gi = 5908455/ug = Mm 38460/len = 359/STRA = for | | | | | | | |
| 9 | 98613_at | Cluster Incl AI849876 UI-M-AL1-aho-h-03-0-UI.s1 Mus musculus cDNA, 3 end/clone = UI-M-AL1-aho-h-03-0-UI/ | P | 172.8 | P | 265.4 | P | 170.9 | P |
| | | clone_end = 3/gb = AI849876/gi = 5493775/ug = Mm 38521/len = 416/STRA = for | | | | | | | |
| 9 | 98615_at | Cluster Incl AW049570 UI-M-BH1-anf-h-02-0-UI s1 Mus musculus cDNA, 3 end/clone = UI-M-BH1-anf-h-02-0-UI/ | P | 810.8 | P | 721.1 | P | 625.7 | P |
| | | clone_end = 3/gb = AW049570/gi = 5910099/ug = Mm 38524/len = 232/STRA = for | | | | | | | |
| 9 | 99594_at | Cluster Incl AI844598 UI-M-AJ1-ahh-c-08-0-UI s2 Mus musculus cDNA, 3 end/clone = UI-M-AJ1-ahh-c-08-0-UI/ | P | 1119.7 | P | 1280.7 | P | 1126.7 | P |
| | | clone_end = 3/gb = AI844598/gi = 5488509/ug = Mm 42826/len = 329/STRA = for | | | | | | | |
| 9 | 99632_at | Cluster Incl U83902 Mus musculus mitotic checkpoint component Mad2 mRNA, complete cds/cds = (48,665)/gb = U83902/ | P | 392.6 | P | 338.8 | P | 178.7 | P |
| | | gi = 4099130/ug = Mm 43444/len = 707/STRA = for | | | | | | | |
| 9 | 99645_at | Cluster Incl AW048484 UI-M-BH1-alj-d-10-0-UI.s1 Mus musculus cDNA, 3 end/clone = UI-M-BH1-adj-d-10-o-UI/ | P | 614 | P | 591.9 | P | 544.5 | P |
| | | clone_end = 3/gb = AW048484/gi = 5909018/ug = Mm 43640/len = 458/STRA = for | | | | | | | |
| 9 | 100113_s_at | Cluster Incl D50367: Mus musculus mRNA for KAP3B, complete cds/cds = (193,2511)/gb = D50367/gi = 1526420/ | P | 256.5 | P | 188.4 | P | 140.3 | P |
| | | ug = Mm 4651/len = 3908/STRA = for | | | | | | | |
| 9 | 100116_at | Cluster Incl AI22538 uc62107 r1 Mus musculus cDNA, 5 end/clone = IMAGE-1430245/clone_end = 5/gb = AI122538/ | P | 729.6 | P | 1214.3 | P | 680.7 | P |
| | | gi = 3522662/ug = Mm.46673/len = 505/STRA = for | | | | | | | |
| 9 | 100522_s_at | Cluster Incl U92454 Mus musculus WW domain binding protein 5 mRNA, partial cds/cds = (0,185)/gb = U92454/ | P | 2122.1 | P | 2431.1 | P | 1734.3 | P |
| | | gi = 1914850/ug = Mm 512/len = 647/STRA = for | | | | | | | |
| 9 | 100523_r_at | Cluster Incl U92454 Mus musculus WW domain binding protein 5 mRNA, partial cds/cds = (0,185)/gb = U92454/ | P | 471.8 | P | 474.7 | P | 408.4 | P |
| | | gi = 1914850/ug = Mm.512/len = 647/STRA = for | | | | | | | |
| 9 | 100588_at | Cluster Incl U60329: Protease (prosome, macropain) 28 subunit, beta/cds = (1,720)/gb = U60329/gi = 1698571/ug = Mm 626/ | P | 1198.3 | P | 1021.9 | P | 565.7 | P |
| | | len = 723/STRA = for | | | | | | | |
| 9 | 101001_at | Cluster Incl AI647612: uk41h01 x1 Mus musculus cDNA, 3 end/clone = IMAGE-1971601/clone_end = 3/gb = AI647612/ | P | 1148.8 | P | 1196.7 | P | 1074.2 | P |
| | | gi = 4726290/ug = Mm.6766/len = 524/STRA = for | | | | | | | |
| 9 | 101002_at | Cluster Incl AF032128 Mus musculus antizyms inhibitor mRNA, complete cds/cds = (16,1362)/gb = AF032128/gi = 2645700/ | P | 515.2 | P | 454.1 | P | 305.5 | P |
| | | ug = Mm.6775/len = 1599/STRA = for | | | | | | | |
| 9 | 101004_f_at | Cluster Incl X91658: Splicing factor, arginine/serine-rich 3 (SRp20)/cds = (116,490)/gb = X91658/gi = 2125862/ug = Mm 6787/ | P | 1113.6 | P | 989.3 | P | 570.5 | P |
| | | len = 1895/STRA = for | | | | | | | |
| 9 | 101056_at | Cluster Incl X60672 M musculus mRNA for radixin/cds = (165,19166)/gb = X60672/gi = 1033049/ug = Mm.7051/len = 4238/ | P | 1084.3 | P | 1068.9 | P | 889.1 | P |
| | | STRA = for | | | | | | | |
| 9 | 101060_at | Cluster Incl M73329: Mouse phospholipase C-alpha (PCL-alpha) mRNA, complete cds/cds = (79,1593)/gb = M73329/ | P | 1275.5 | P | 1301.1 | P | 840.2 | P |
| | | gi = 200396/ug = Mm.709/len = 1900/STRA = for | | | | | | | |
| 9 | 101064_at | Cluster Incl AF044334: Mus musculus plelotropic regulator 1 (PLRG1) mRNA, complete cds/cds = (0,1541)/gb = AF044334/ | P | 615.8 | P | 727.3 | P | 553.2 | P |
| | | gi = 2832297/ug = Mm.7138/len = 1542/STRA = for | | | | | | | |
| 9 | 101065_at | Cluster Incl X57800: Proliferating cell nuclear antigen/cds = (144,929)/gb = X57800/gi = 53601/ug = Mm.7141/len = 1261/ | P | 1808.3 | P | 1473.8 | P | 815.2 | P |
| | | STRA = for | | | | | | | |
| 9 | 101067_at | Cluster Incl AW124711: UI-M-BH2 1-aqa-a-12-0-UI.s1 Mus musculus cDNA, 3 end/clone = UI-M-BH2.1-aqa-a-12-0-UI/ | P | 849 | P | 1031.4 | P | 794.9 | P |
| | | clone_end = 3/gb = AW124711/gi = 6100241/ug = Mm 719/len = 467/STRA = for | | | | | | | |
| 9 | 101091_at | Cluster Incl AF033353 Ubiquitin-like 1/cds = (118,423)/gb = AF033353/gi = 2645736/ug = Mm.7353/len = 1187/STRA = for | P | 1291.8 | P | 977.1 | P | 642.4 | P |
| 9 | 101506_at | Cluster Incl AW227345 up 11a09 y1 Mus musculus cDNA, 5 end/clone = IMAGE-2651704/clone_end = 5/gb = AW227345/ | P | 424.1 | P | 428.1 | P | 257.5 | P |
| | | gi = 6556710/ug = Mm 821/len = 447/STRA = for | | | | | | | |
| 9 | 101509_at | Cluster Incl U96760 Mus musculus von Hippel-Lindau binding protein homolog (vbp-1) mRNA, complete cds/ | P | 800.5 | P | 684.1 | P | 610.6 | P |
| | | cds = (101,583)/gb = U96760/gi = 2738245/ug = Mm 8294/len = 1531/STRA = for | | | | | | | |
| 9 | 101580_at | Cluster Incl AI851220 UI-M-BH0-ejx-g-09-0-UI s1 Mus musculus cDNA, 3 end/clone = UI-M-BH0-ajx-g-09-0-UI/ | P | 2223.6 | P | 2406.2 | P | 1682.1 | P |
| | | clone_end = 3/gb = AI851220/gi = 5495126/ug = Mm.90007/len = 344/STRA = for | | | | | | | |
| 9 | 101958_f_at | Cluster Incl X72310: Transcription factor Dp 1/cds = (54,1286)/gb = X72310/gi = 395280/ug = Mm 925/len = 1700/STRA = for | P | 604 | P | 623.8 | P | 446.1 | P |
| 9 | 101959_r_at | Cluster Incl X72310 Transcription factor Dp 1/cds = (54,1286)/gb = X72310/gi = 395280/ug = Mm 925/len = 1700/STRA = for | P | 942.3 | P | 884.8 | P | 661.5 | P |
| 9 | 102001_at | Cluster Incl M14223 Mouse ribonucleotide reductase M2 subunit mRNA, complete cds/cds = (62,1234)/gb = M14223/ | P | 388.6 | P | 431.8 | P | 297.8 | P |
| 9 | 102409_at | Cluster Incl AW046963 UI-M-BH1-alp-c-c7-0-UI s1 Mus musculus cDNA, 3 end/clone = UI-M-BH1-alp-c-07-0-UI/ | P | 634.2 | P | 567.5 | P | 297.5 | P |
| | | clone_end = 3/gb = AW046963/gi = 5907492/ug = Mm 12539/len = 307/STRA = for | | | | | | | |
| 9 | 102776_at | Cluster Incl AI55095 ud59b08 r1 Mus musculus cDNA, 5 end/clone = IMAGE-1450163/clone_end = 5/gb = AI55095/ | P | 262.5 | P | 234.9 | P | 143.6 | P |
| | | gi = 3683564/ug = Mm 13437/len = 120/STRA = for | | | | | | | |
| 9 | 103036_at | Cluster incl M38700 Thyroid autoantigen 70 kDa/cds = (0,1778)/gb = M38700/gi = 198636/ug = Mm.16147/len = 1944/ | P | 177.5 | P | 188.7 | P | 103.6 | P |
| | | STRA = for | | | | | | | |
| 9 | 103342_at | Cluster Incl U78103: Embryonic ectoderm development/cds = (453,1778)/gb = U76103/gi = 1710987/ug = Mm 18203/ | P | 373.3 | P | 265.9 | P | 184.2 | P |
| | | len = 2040/STRA = for | | | | | | | |
| 9 | 103402_at | Cluster Incl AI848522: UI-M-AP1-agf-e-06-0-UI s2 Mus musculus cDNA, 3 end/clone = UI-M-AP1-agf-e-06-0-UI/ | P | 298.5 | P | 327.2 | P | 273.4 | P |
| | | clone_end = 3/gb = AI848522/gi = 5492428/ug = Mm.18761/len = 425/STRA = for | | | | | | | |
| 9 | 103406_at | Cluster Incl AI853967 UI-M-BH0-aiv-e-02-0-UI s1 Mus musculus cDNA, 3 end/clone = UI-M-BH0-aiv-e-02-0-UI/ | P | 287.3 | P | 238.1 | P | 268.9 | P |
| | | clone_end = 3/gb = AI853967/gi = 5497873/ug = Mm 18798/len = 434/STRA = for | | | | | | | |
| 9 | 103418_at | Cluster Incl AW122092: UI-M-BH2.2-aoo-g-10-0-UI s1 Mus musculus cDNA, 3 end/clone = UI-M-BH2 2-aoo-g-10-0-UI/ | P | 264.4 | P | 286.4 | P | 183.1 | P |
| | | clone_end = 3/gb = AW122092/gi = 6097499/ug = Mm.18876/len = 451/STRA = for | | | | | | | |
| 9 | 103852_at | Cluster Incl AI848207 UI-M-AH1-agq-g-08-0-UI s1 Mus musculus cDNA, 3 end/clone = UI-M-AH1-agq-g-08-0-UI/ | P | 214.9 | P | 206.1 | P | 161 | P |
| | | clone_end = 3/gb = AI848207/gi = 5492118/ug = Mm 21064/len = 455/STRA = for | | | | | | | |
| 9 | 103873_i_at | Cluster Incl AA684456: vm64c06.s1 Mus musculus cDNA, 5 end/clone = IMAGE-1003018/clone_end = 5/gb = AA684456/ | P | 288.2 | P | 230.8 | P | 195.1 | P |
| | | gi = 2671042/ug = Mm.21173/len = 217/STRA = for | | | | | | | |
| 9 | 103876_at | Cluster Incl AA760339: vv75c06.r1 Mus musculus cDNA, 5 end/clone = IMAGE-1228234/clone_end = 5/gb = AA760339/ | P | 694.8 | P | 601.1 | P | 558.9 | P |
| | | gi = 2808133/ug = Mm.21181/len = 552/STRA = for | | | | | | | |
| 9 | 103881_at | Cluster Incl AA691185: vt34b11.r1 Mus musculus cDNA, 5 end/clone = IMAGE-1184957/clone_end = 5/gb = AA691185/ | P | 303.6 | P | 271.1 | P | 238.7 | P |
| | | gi = 2692121/ug = Mm.212/len = 184/STRA = for | | | | | | | |
| 9 | 103886_at | Cluster Incl AI850356: UI-M-BG1-aig-a-12-0-UI s1 Mus musculus cDNA, 3 end/clone = UI-M-BG1-aig-a-12-0-UI/ | P | 593.9 | P | 472.4 | P | 413.3 | P |
| | | clone_end = 3/gb = AI850356/gi = 5494262/ug = Mm.21251/len = 443/STRA = for | | | | | | | |
| 9 | 104038_at | Cluster Incl AI843511 UI-M-AO1-aeh-b-11-0-UI a1 Mus musculus cDNA, 3 end/clone = UI-M-AO1-aeh-b-11-0-UI/ | P | 409.8 | P | 489.4 | P | 418.4 | P |
| | | clone_end = 3/gb = AI843511/gi = 5477724/ug = Mm.21451/len = 459/STRA = for | | | | | | | |
| 9 | 104052_at | Cluster Incl AI840921: UI-M-AMO-adm-f-12-0-UI s1 Mus musculus cDNA, 3 end/clone = UI-M-AM0-adm-f-12-0-UI/ | P | 273.4 | P | 215.2 | P | 241.6 | P |
| | | clone_end = 3/gb = AI840921/gi = 5475134/ug = Mm.21520/len = 204/STRA = for | | | | | | | |
| 9 | 104077_at | Cluster Incl AW121992: UI-M-BH2.3-aoj-f-11-0-UI s1 Mus musculus cDNA, 3 end/clone = UI-M-BH2.3-aoj-f-11-0-UI/ | P | 359.6 | P | 360.1 | P | 279.5 | P |
| | | clone_end = 3/gb = AW121992/gi = 6097398/ug = Mm 21705/len = 477/STRA = for | | | | | | | |
| 9 | 104090_at | Cluster Incl AA657164: vr26f01 r1 Mus musculus cDNA, 5 end/clone = IMAGE-1121785/clone_end = 5/gb = AA657164/ | P | 225.4 | P | 253.4 | P | 211.2 | P |
| | | gi = 2593318/ug = Mm.21803/len = 376/STRA = for | | | | | | | |
| 9 | 104126_at | Cluster Incl AI854864: UI-M-BHO-akb-e-01-0-UI a1 Mus musculus cDNA, 3 end/clone = UI-M-BHO-akb-e-01-0-UI/ | P | 271.2 | P | 267.2 | P | 281.7 | P |
| | | clone_end = 3/gb = AI854864/gi = 5498770/ug = Mm.22031/len = 197/STRA = for | | | | | | | |
| 9 | 104134_at | Cluster Incl Y17851: Mus musculus mRNA for ganglioside-induced differentiation associated protein 2/cds = (0,223)/ | P | 172.3 | P | 192.8 | P | 177.3 | P |
| | | gb = Y17851/gi = 3378455/ug = Mm.22080/len = 197/STRA = for | | | | | | | |
| 9 | 104305_at | Cluster Incl AW120679: UI-M-BH2.3-anz-c-02-0-UI s1 Mus musculus cDNA, 3 end/clone = UI-M-BH2.3-anz-c-02-0-UI/ | P | 561.4 | P | 474.8 | P | 318.1 | P |
| | | clone_end = 3/gb = AW120679/gi = 6095977/ug = Mm.22363/len = 454/STRA = for | | | | | | | |
| 9 | 104378_at | Cluster Incl L4851: Paraoxonase 2/cds = (24,1088)/gb = L48514/gi = 1333637/ug = Mm.22581/len = 1179/STRA = for | P | 250.1 | P | 209.6 | P | 135.8 | P |
| 9 | 104527_at | Cluster Incl D13803: Rad51 homolog (S cerevisiae)/cds = (188,1207)/gb = D13803/gi = 303702/ug = Mm.231/len = 1408/ | P | 263.8 | P | 272.1 | P | 252.9 | P |
| | | STRA = for | | | | | | | |
| 9 | 104544_at | Cluster Incl AA675604: vr73b04.s1 Mus musculus cDNA, 5 end/clone = IMAGE-1134319/clone_end = 5/gb = AA675604/ | P | 261.2 | P | 291.2 | P | 244.5 | P |
| | | gi = 2652841/ug = Mm.23690/len = 469/STRA = for | | | | | | | |
| 9 | 104565_at | Cluster Incl AI847561: UI-M-AM1-afv-g-05-0-UI s1 Mus musculus cDNA, 3 end/clone = UI-M-AM1-afv-g-05-0-UI/ | P | 320.9 | P | 329.3 | P | 345.7 | P |
| | | clone_end = 3/gb = AI847561/gi = 5491467/ug = Mm.23861/len = 370/STRA = for | | | | | | | |
| 9 | 104586_at | Cluster Incl AI842878: UI-M-AN1-afb-e-0-UI.s1 Mus musculus cDNA, 3 end/clone = UI-M-AN1-afb-e-12-0-UI/ | P | 291.1 | P | 225.4 | P | 187.6 | P |
| | | clone_end = 3/gb = AI842878/gi = 5477091/ug = Mm.23985/len = 162/STRA = for | | | | | | | |
| 9 | 104634_at | Cluster Incl AA874589: vx78a06 r1 Mus musculus cDNA, 5 end/clone = IMAGE-1281298/clone_end = 5/gb = AA874589/ | P | 798 | P | 592.4 | P | 569.5 | P |
| | | gi = 2979278/ug = Mm.24345/len = 559/STRA = for | | | | | | | |
| 9 | 104735_at | Cluster Incl AI842065: UI-M-AN1-afg-a-10-0-UI.s1 Mus musculus cDNA, 3 end/clone = UI-M-AN1-afg-a-10-0-UI/ | P | 503.6 | P | 321.9 | P | 282.5 | P |
| | | clone_end = 3/gb = AI842065/gi = 5476278/ug = Mm.24647/len = 444/STRA = for | | | | | | | |
| 9 | 104740_at | Cluster Incl AW125318: UI-M-BH2.1-apy-a-04-0-UI.s1 Mus musculus cDNA, 3 end/clone = UI-M-BH2.1-apy-a-04-0-UI/ | P | 233.6 | P | 245.7 | P | 123.2 | P |
| | | clone_end = 3/gb = AW125318/gi = 6100848/ug = Mm.24671/len = 405/STRA = for | | | | | | | |
| 9 | 104766_at | Cluster Incl AI851198: UI-M-BH0-ajx-e-09-0-UI.s1 Mus musculus cDNA, 3 end/clone = UI-M-BH0-ajx-e-09-0-UI/ | P | 762.9 | P | 737.5 | P | 571.9 | P |
| | | clone_end = 3/gb = AI851198/gi = 5495104/ug = Mm.24873/len = 193/STRA = rev | | | | | | | |
| 9 | 94346_at | Cluster Incl AI853721: UI-M-BH0-ajq-e-02-0-UI.s1 Mus musculus cDNA, 3 end/clone = UI-M-BH0-ajq-e-02-0-UI/ | P | 178.8 | P | 136.8 | P | 135.9 | P |
| | | clone_end = 3/gb = AI853721/gi = 5497592/ug = Mm.25154/len = 203/STRA = for | | | | | | | |
| 9 | 94966_at | Cluster Incl Z11911: Glucose-6-phosphate dehydrogenase X-linked/cds = (52,1599)/gb = Z11911/gi = 51113/ug = Mm.27210/ | P | 812.9 | P | 919.6 | P | 494.9 | P |
| | | len = 2322/STRA = for | | | | | | | |
| 9 | 94967_at | Cluster Incl AI851365: UI-M-BH0-akh-e-08-0-UI.s1 Mus musculus cDNA, 3 end/clone = UI-M-BH0-akh-e-08-0-UI/ | P | 421.2 | P | 376.6 | P | 352.7 | P |
| | | clone_end = 3/gb = AI851365/gi = 5495271/ug = Mm.27216/len = 481/STRA = for | | | | | | | |
| 9 | 94982_f_at | Cluster Incl AI852470: UI-M-BH0-alp-f-08-0-UI.s1 Mus musculus cDNA, 3 end/clone = UI-M-BH0-alp-f-08-0-UI/ | P | 239.1 | P | 333.9 | P | 149.9 | P |
| | | clone_end = 3/gb = AI852470/gi = 5496376/ug = Mm.27278/len = 422/STRA = for | | | | | | | |
| 9 | 94989_at | Cluster Incl AI848984: UI-M-AJ1-ahd-d-06-0UI.s1 Mus musculus cDNA, 3 end/clone = UI-M-AJ1-ahd-d-06-0-UI/ | P | 988.4 | P | 1024.8 | P | 722 | P |
| | | clone_end = 3/gb = AI848984/gi = 5492890/ug = Mm.27311/len = 476/STRA = for | | | | | | | |
| 9 | 94998_at | Cluster Incl AI838452: UI-M-AJ0-aba-e-08-0-UI.s1 Mus musculus cDNA, 3 end/clone = UI-M-AJ0-aba-e-08-0-UI/ | P | 175.9 | P | 174.9 | P | 135.1 | P |
| | | clone_end = 3/gb = AI838452/gi = 5472665/ug = MM.27348/len = 466/STRA = for | | | | | | | |
| 9 | 95003_at | Cluster Incl AA80275: vv99ho2.r1 Mus musculus cDNA, 5 end/clone = IMAGE-1230579/clone_end = 5/gb = AA880275/ | P | 541.7 | P | 467.3 | P | 448.2 | P |
| | | gi = 2989258/ug = Mm.27375/len = 382/STRA = for | | | | | | | |
| 9 | 95018_r_at | Cluster Incl AA607053: vm95e01.r1 Mus musculus cDNA, 5 end/clone = IMAGE-1006008/clone_end = 5/gb = AA607053/ | P | 375.6 | P | 505 | P | 392.6 | P |
| | | gi = 2455946/ug = Mm.27454/len = 178/STRA = for | | | | | | | |
| 9 | 95614_at | Cluster Incl AI852086: UI-M-BH0-aja-g-03-0-UI.s1 Mus musculus cDNA, 3 end/clone = UI-M-BH0-aja-g-03-0-UI/ | P | 430 | P | 499 | P | 419.7 | P |
| | | clone_end = 3/gb = AI852086/gi = 5495992/ug = Mm.28003/len = 439/STRA = for | | | | | | | |
| 9 | 96158_at | Cluster Incl AW123477: UI-M-BH2.1-apj-g-12-0-UI.s1 Mus musculus cDNA, 3 end/clone = UI-M-BH2.1-apj-g-12-0-UI/ | P | 414.5 | P | 436.9 | P | 473.6 | P |
| | | clone_end = 3/gb = AW123477/gi = 6099007/ug = Mm.28332/len = 423/STRA = for | | | | | | | |
| 9 | 96168_at | Cluster Incl AI591702: vq88c03.y1 Mus musculus cDNA, 5 end/clone = IMAGE-1109380/clone_end = 5/gb = AI591702/ | P | 331 | P | 461.1 | P | 89.5 | P |
| | | gi = 4800750/ug = Mm.28386/len = 266/STRA = for | | | | | | | |
| 9 | 96743_at | Cluster Incl AA958560: ua14109.r1 Mus musculus cDNA, 5 end/clone = IMAGE-1346729/clone_end = 5/gb = AA958560/ | P | 716.8 | P | 667.3 | P | 465.2 | P |
| | | gi = 3124790/ug = Mm.28938/len = 292/STRA = for | | | | | | | |
| 9 | 97327_at | Cluster Incl L26320: Flap structure specific endonuclease 1/cds = (103,1239)/gb = L26320/gi = 499648/ug = Mm.2952/ | P | 314.4 | P | 412.3 | P | 259.4 | P |
| | | len = 2033/STRA = for | | | | | | | |
| 9 | 97329_at | Cluster Incl AW124061: UI-M-BH2.1-apn-f-03-0-UI.s1 Mus musculus cDNA, 3 end/clone = UI-M-BH2.1-apn-f-03-0-UI/ | P | 232.2 | P | 245.2 | P | 217.5 | P |
| | | clone_end = 3/gb = AW124061/gi = 6099591/ug = Mm.29529/len = 437/STRA = for | | | | | | | |
| 9 | 97411_at | Cluster Incl L11316: Ect2 oncogene/cds = (530,2746)/gb = L11316/gi = 293331/ug = Mm 2995/len = 3920/STRA = for | P | 369.3 | P | 333.5 | P | 195 | P |
| 9 | 97412_at | Cluster Incl AI843157: UI-M-AK1-aet-a-08-0-UI.s1 Mus musculus cDNA, 3 end/clone = UI-M-AK1-aet-a-08-0-UI/ | P | 440.1 | P | 630.5 | P | 284.9 | P |
| | | clone_end = 3/gb = AI843157/gi = 5477370/ug = Mm 29952/len = 443/STRA = for | | | | | | | |
| 9 | 97425_at | Cluster Incl AI840191: UI-M-AO0-acc-g-09-0-UI.s1 Mus musculus cDNA, 3 end/clone = UI-M-AO0-acc-g-09-0-UI/ | P | 501.2 | P | 612.9 | P | 370.8 | P |
| | | clone_end = 3/gb = AI840191/gi = 5474404/ug = Mm.30015/len = 423/STRA = for | | | | | | | |
| 9 | 97916_at | Cluster Incl AI116222: uc18c11.r1 Mus musculus cDNA, 5 end/clone = IMAGE-1398356/clone_end = 5/gb = AI116222/ | P | 570.5 | P | 603.6 | P | 411.4 | P |
| | | gi = 3516546/ug = Mm30116/len = 321/STRA = rev | | | | | | | |
| 9 | 97922_at | Cluster Incl AW045597: UI-M-BH1-alg-a-01-0-UIs1 Mus musculus cDNA, 3 end/clone = UI-M-BH1-alg-a-01-0-UI/ | P | 754.9 | P | 767.9 | P | 679.5 | P |
| | | clone_end = 3/gb = AW045597/gi = 5906121/ug = Mm30166/len = 306/STRA = for | | | | | | | |
| 9 | 98451_at | Cluster Incl AI843164: UI-M-AK1-aet-b-03-0-UIs1 Mus musculus cDNA, 3 end/clone = UI-M-AK1-aet-b-03-0-UI/ | P | 191.8 | P | 167.5 | P | 151 | P |
| | | clone_end = 3/gb = AI843164/gi = 5477377/ug = Mm34635/len = 375/STRA = rev | | | | | | | |
| 9 | 98463_at | Cluster Incl AW124186: UI-M-BH2.1-aph-a-05-0-UIs1 Mus musculus cDNA, 3 end/clone = UI-M-BH2.1-aph-a-05-0-UI/ | P | 730.4 | P | 810.5 | P | 530.5 | P |
| | | clone_end = 3/gb = AW124186/gi = 6099716/ug = Mm34803/len = 237/STRA = for | | | | | | | |
| 9 | 98996_at | Cluster Incl L29480 Mus musculus serine/threonine kinase (sak-b) mRNA, complete cds/cds = (205,1599)/gb = L29480/ | P | 425.2 | P | 291.8 | P | 152.6 | P |
| | | gi = 487871/ug = Mm3794/len = 1600/STRA = for | | | | | | | |
| 9 | 99440_at | Cluster Incl Y07686 M musculus mRNA for NfiB3-protein, splice vanant (exon 9, 10 and 11 missing)/cds = (531,1793)/ | P | 1093.8 | P | 1761 | P | 1330.4 | P |
| | | gb = Y07686/gi = 1524158/ug = Mm4025/len = 2514/STRA = rev | | | | | | | |
| 9 | 99457_at | Cluster Incl X82786 Antigen identified by monoclonal antibody Ki 67/cds = (345,9161)/gb = X82786/gi = 1177527/ | P | 1178.1 | P | 1122.1 | P | 270.2 | P |
| | | ug = Mm4078/len = 9471/STRA = for | | | | | | | |
| 9 | 99962_at | Cluster Incl D12644: Kinesin heavy chain member 2/cds = (504,2654)/gb = D12644/gi = 220467/ug = Mm4415/len = 3532/ | P | 149.1 | P | 119.9 | P | 101 | P |
| | | STRA = for | | | | | | | |
| 9 | 100033_at | Cluster Incl X81143 MutS homolog 2 (E. coli)/cds = (39,2846)/gb = X81143/gi = 534944/ug = Mm4619/len = 3079/STRA = for | P | 497.2 | P | 529.4 | P | 481.7 | P |
| 9 | 100460_at | Cluster Incl L49502: TPR-containing, SH2-binding phosphoprotein/cds = (134,3655)/gb = L49502/gi = 1236238/ug = Mm489/ | P | 215.9 | P | 182.7 | P | 165.8 | P |
| | | len = 4201/STRA = rev | | | | | | | |
| 9 | 100892_at | Cluster Incl AI850186 UI-M-BG1-aid-h-04-0-UIs1 Mus musculus cDNA, 3 end/clone = UI-M-BG1-aid-h-04-0-UI/ | P | 158 | P | 190.6 | P | 157.7 | P |
| | | clone_end = 3/gb = AI850186/gi = 5494092/ug = Mm5545/len = 271/STRA = for | | | | | | | |
| 9 | 100979_at | Cluster Incl AW209763 ul46g10 y1 Mus musculus cDNA, 5 end/clone = IMAGE-2101410/clone_end = 5/gb = AW209763/ | P | 285.7 | P | 221.2 | P | 151.2 | P |
| | | gi = 6515703/ug = Mm6703/len = 440/STRA = for | | | | | | | |
| 9 | 101367_at | Cluster Incl U60312: Dynactin 1/cds = (19,3864)/gb = U60312/gi = 2104494/ug = Mm6919/len = 4024/STRA = for | P | 443.3 | P | 481 | P | 429 | P |
| 9 | 101439_at | Cluster Incl AW122716 UI-M-BH22-aot-d-03-0-UIs1 Mus musculus cDNA, 3 end/clone = UI-M-BH22-aot-d-03-0-UI/ | P | 271.4 | P | 273.6 | P | 115.5 | P |
| | | clone_end = 3/gb = AW122716/gi = 6098246/ug = Mm7760/len = 409/STRA = for | | | | | | | |
| 9 | 102883_at | Cluster Incl AW107702 um33b01 x1 Mus musculus cDNA, 3 end/clone = IMAGE-2236297/clone_end = 3/gb = AW107702/ | P | 115.8 | P | 154.6 | P | 153 | P |
| | | gi = 6078502/ug = Mm1506/len = 548/STRA = for | | | | | | | |
| 9 | 104002_at | Cluster Incl AI153693 vz91d04 r1 Mus musculus cDNA, 5 end/clone = IMAGE-1344583/clone_end = 5/gb = AI153693/ | P | 102.3 | P | 111.7 | P | 57.4 | P |
| | | gi = 3682162/ug = Mm26595/len = 400/STRA = for | | | | | | | |
| 9 | 104030_at | Cluster Incl AI848841 UI-M-AJ1-ahb-c-08-0-UIs1 Mus musculus cDNA, 3 end/clone = UI-M-AJ1-ahb-c-08-0-UI/ | P | 1252.3 | P | 1196.8 | P | 1047.1 | P |
| | | clone_end = 3/gb = AI848841/gi = 5492747/ug = Mm27042/len = 249/STRA = for | | | | | | | |
| 9 | 104644_at | Cluster Incl D12646 Kinesin heavy chain member 4/cds = (731,4426)/gb = D12646/gi = 563772/ug = Mm3130/len = 4706/ | P | 184.8 | P | 151.6 | P | 106 | P |
| | | STRA = for | | | | | | | |
| 9 | 92526_f_at | Cluster Incl AW122114 UI-M-BH22-aor-a-11-0-UIs1 Mus musculus cDNA, 3 end/clone = UI-M-BH22-aor-a-11-0-UI/ | P | 97.8 | P | 117.7 | P | 74.5 | P |
| | | clone_end = 3/gb = AW122114/gi = 6097531/ug = Mm4285/len = 271/STRA = for | | | | | | | |
| 9 | 93236_s_at | Cluster Incl M13352 Thymidylate synthase/cds = (0,923)/gb = M13352/gi = 202049/ug = Mm5879/len = 924/STRA = for | P | 246.4 | P | 375.3 | P | 158.6 | P |
| 9 | 93859_at | Cluster Incl AI875598: uk50c11 x1 Mus musculus cDNA, 3 end/clone = IMAGE-1972436/clone_end = 3/gb = AI875598/ | P | 333.2 | P | 301.1 | P | 144.5 | P |
| | | gi = 5549647/ug = Mm87729/len = 513/STRA = rev | | | | | | | |
| 9 | 95914_at | Cluster Incl AA177382: mt29b12.r1 Mus musculus cDNA, 5 end/clone = IMAGE-622463/clone_end = 5/gb = AA177382/ | P | 157.1 | P | 136.3 | P | 148.6 | P |
| | | gi = 1758617/ug = Mm24464/len = 542/STRA = for | | | | | | | |
| 9 | 96594_at | Cluster Incl AA615831 vo90h05 r1 Mus musculus cDNA, 5 end/clone = IMAGE-1066425/clone_end = 5/gb = AA615831/ | P | 631.1 | P | 758 | P | 303.4 | P |
| | | gi = 2503059/ug = Mm30652/len = 257/STRA = for | | | | | | | |
| 9 | 97164_at | Cluster Incl C77566 C77566 Mus musculus cDNA, 3 end/clone = J0033G09/clone_end = 3/gb = C77566/gi = 2517896/ | P | 419.6 | P | 421 | P | 347.6 | P |
| | | ug = Mm35516/len = 616/STRA = rev | | | | | | | |
| 9 | 99366_at | Cluster Incl AI553536: vw39e06.x1 Mus musculus cDNA, 3 end/clone = IMAGE-1246210/clone_end = 3/gb = AI553536/ | P | 379.5 | P | 192.8 | P | 181.6 | P |
| | | gi = 4485899/ug = Mm5675/len = 408/STRA = rev | | | | | | | |
| 9 | 102144_f_at | Cluster Incl AW048189 UI-M-BH1-amc-d-12-0-UIs1 Mus musculus cDNA, 3 end/clone = UI-M-BH1-amc-d-12-0-UI/ | P | 248.4 | P | 269.1 | P | 184.5 | P |
| | | clone_end = 3/gb = AW048189/gi = 5908718/ug = Mm44453/len = 141/STRA = rev | | | | | | | |
| 9 | 93992_at | Cluster Incl AW123342: UI-M-BH2.1-apf-d-04-0-UIs1 Mus musculus cDNA, 3 end/clone = UI-M-BH2.1-apf-d-04-0-UI/ | P | 281.7 | P | 176.2 | P | 9.8 | P |
| | | clone_end = 3/gb = AW123342/gi = 6098872/ug = Mm21749/len = 405/STRA = for | | | | | | | |
| 9 | 95447_at | Cluster Incl AA683761 vr04e08 r1 Mus musculus cDNA, 5 end/clone = IMAGE-1110854/clone_end = 5/gb = AA683761/ | P | 317.7 | P | 275.4 | P | 312.9 | P |
| | | gi = 2670347/ug = Mm24601/len = 335/STRA = for | | | | | | | |
| 9 | 96002_at | Cluster Incl AW123936: UI-M-BH2.3-aqc-c-01-0-UIs1 Mus musculus cDNA, 3 end/clone = UI-M-BH2.3-aqc-c-01-0-UI/ | P | 333 | P | 314.1 | P | 258.7 | M |
| | | clone_end = 3/gb = AW123936/gi = 6099466/ug = Mm25331/len = 368/STRA = for | | | | | | | |
| 9 | 96654_at | Cluster Incl AI852916: UI-M-BH0-aiz-b-03-0-UIs1 Mus musculus cDNA, 3 end/clone = UI-M-BH0-aiz-b-03-0-UI/ | P | 92 | P | 78.8 | P | 60 | P |
| | | clone_end = 3/gb = AI852916/gi = 5496822/ug = Mm27339/len = 392/STRA = for | | | | | | | |
| 9 | 97261_at | Cluster Incl AF055664: Heat shock protein, DNAJ-like 2/cds = (91,1284)/gb = AF055664/gi = 3025863/ug = Mm27897/ | P | 615.2 | P | 441.2 | P | 323.8 | A |
| | | len = 2242/STRA = for | | | | | | | |
| 9 | 94447_at | Cluster Incl AA726837 vu94h01 r1 Mus musculus cDNA, 5 end/clone = IMAGE-1209841/clone_end = 5/gb = AA726837/ | P | 45.3 | P | 275.7 | P | 85.5 | P |
| | | gi = 2744544/ug = Mm26780/len = 654/STRA = for | | | | | | | |
| 9 | 95075_at | Cluster Incl AW229141 up29a12 y1 Mus musculus cDNA, 5 end/clone = IMAGE-2655742/clone_end = 5/gb = AW229141/ | P | 225.1 | P | 215.8 | P | 208.3 | A |
| | | gi = 6558373/ug = Mm29200/len = 287/STRA = for | | | | | | | |
| 9 | 95712_at | Cluster Incl AW045261 UI-M-BH1-akn-a-07-0-UI s1 Mus musculus cDNA, 3 end/clone = UI-M-BH1-akn-a-07-0-UI/ | P | 241.1 | P | 175.4 | P | 164.7 | A |
| | | clone_end = 3/gb = AW045261/gi = 5905790/ug = Mm29709/len = 301/STRA = for | | | | | | | |
| 9 | 99149_at | Cluster Incl AI851230 UI-M-BH0-ajx-h-10-0-UIs1 Mus musculus cDNA, 3 end/clone = UI-M-BH0-ajx-h-10-0-UI/ | P | 269.6 | P | 338.9 | P | 115.4 | P |
| | | clone_end = 3/gb = AI851230/gi = 5495136/ug = Mm41535/len = 416/STRA = for | | | | | | | |
| 9 | 99616_s_at | Cluster Incl AI842156 UI-M-AN1-afj-e-11-0-UIs1 Mus musculus cDNA, 3 end/clone = UI-M-AN1-afj-e-11-0-UI/ | P | 416.5 | P | 274.8 | P | 238.2 | A |
| | | clone_end = 3/gb = AI842156/gi = 5476369/ug = Mm43017/len = 316/STRA = for | | | | | | | |
| 9 | 100093_at | Cluster Incl X69025 PCTAIRE-motif protein kinase 1/cds = UNKNOWN/gb = X69025/gi = 53610/ug = Mm4597/len = 3078/ | P | 904.4 | P | 789.1 | P | 774.7 | P |
| | | STRA = for | | | | | | | |
| 9 | 100128_at | Cluster Incl M38724: Cell division cycle control protein 2a/cds = (24,917)/gb = M38724/gi = 192533/ug = Mm.4761/len = 963/ | P | 486.2 | P | 293.6 | P | 55.2 | A |
| | | STRA = for | | | | | | | |
| 9 | 101481_at | Cluster Incl AI852555: UI-M-BH0-aiw-g-04-0-UI.s1 Mus musculus cDNA, 3 end/clone = UI-M-BH0-aiw-g-04-0-UI/ | P | 151 | P | 212.8 | P | 148.1 | P |
| | | clone_end = 3/gb = AI852555/gi = 5496461/ug = Mm.7780/len = 351/STRA = for | | | | | | | |
| 9 | 103582_r_at | Cluster Incl AI845633 UI-M-AQ1-adx-h-07-0-UI s1 Mus musculus cDNA, 3 end/clone = UI-M-AQ1-adx-h-07-0-UI/ | P | 204.5 | P | 240.9 | P | 148.8 | A |
| | | clone_end = 3/gb = AI845633/gi = 5489539/ug = Mm.19799/len = 430/STRA = for | | | | | | | |
| 9 | 104373_at | Cluster Incl AW046876 UI-M-BH1-aid-c-02-0-UI s1 Mus musculus cDNA, 3 end/clone = UI-M-BH1-aki-c-02-0-UI/ | P | 84.9 | P | 79.9 | P | 42.9 | P |
| | | clone_end = 3/gb = AW046876/gi = 5907405/ug = Mm 22645/len = 525/STRA = for | | | | | | | |
| 9 | 104547_at | Cluster Incl J00388 mouse dihydrofolate reductase gene/cds = (0,563)/gb = J00388/gi = 193340/ug = Mm 23695/len = 564/ | P | 71.9 | P | 104.8 | P | 88.3 | P |
| | | STRA = for | | | | | | | |
| 9 | 94396_at | Cluster Incl AA647507: vq78d10 s1 Mus musculus cDNA, 5 end/clone = IMAGE-1108435/clone_end = 5/gb = AA647507/ | P | 463.2 | P | 520.6 | P | 331.8 | A |
| | | gi = 2573936/ug = Mm25709/len = 345/STRA = for | | | | | | | |
| 9 | 95523_at | Cluster Incl AI839718 UI-M-AN0-acp-h-02-0-UI s1 Mus musculus cDNA, 3 end/clone = UI-M-AN0-acp-h-02-0-UI/ | P | 549.1 | P | 393.4 | P | 210.4 | P |
| | | clone_end = 3/gb = AI839718/gi = 5473931/ug = Mm.27579/len = 244/STRA = for | | | | | | | |
| 9 | 97926_s_at | Cluster Incl U10374 Peroxisome proliferator activator receptor gamma/cds = (0,1427)/gb = U10374/gi = 507776/ | P | 253.8 | P | 164.3 | P | 92.4 | P |
| | | ug = Mm 3020/len = 1428/STRA = for | | | | | | | |
| 9 | 99917_at | Cluster Incl U52951 Mus musculus putative transcriptional regulator mEnx-1 mRNA, complete cds/cds = (134,2374)/ | P | 302.3 | P | 358.6 | P | 197.7 | P |
| | | gb = U52951/gi = 1279908/ug = Mm4303/len = 2653/STRA = for | | | | | | | |
| 9 | 102907_at | Cluster Incl AW125043: UI-M-BH2 1-apx-b-03-0-UI s1 Mus musculus cDNA, 3 end/clone = UI-M-BH2 1-apx-b-03-0-UI/ | P | 296.9 | P | 135.7 | P | 168 | P |
| | | clone_end = 3/gb = AW125043/gi = 6100573/ug = Mm158/len = 465/STRA = for | | | | | | | |
| 9 | 102934_s_at | Cluster Incl L16926 Cell division cycle control protein 25C/cds = (481,1878)/gb = L16926/gi = 451133/ug = Mm16857/ | P | 213.9 | P | 210.5 | P | 123.5 | P |
| | | len = 2124/STRA = for | | | | | | | |
| 9 | 103696_r_at | Cluster Incl AW047329 UI-M-BH1-amh-h-04-0-UI s1 Mus musculus cDNA, 3 end/clone = UI-M-BH1-amh-h-04-0-UI/ | P | 58 | P | 65.6 | P | 26.4 | P |
| | | clone_end = 3/gb = AW047329/gi = 5907858/ug = Mm.23994/len = 438/STRA = for | | | | | | | |
| 9 | 103726_at | Cluster Incl AI842264 UI-M-AI1-afp-g-06-0-UI s1 Mus musculus cDNA, 3 end/clone = UI-M-AI1-afp-g-06-0-UI/clone_end = 3/ | P | 361.6 | P | 395.3 | P | 242.3 | M |
| | | gb = AI842264/gi = 5476477/ug = Mm24286/len = 384/STRA = for | | | | | | | |
| 9 | 104194_at | Cluster Incl AF082567 Mus musculus hephaestin (heph) mRNA, complete cds/cds = (619,4092)/gb = AF082567/gi = 4322373/ | P | 133.3 | P | 58.7 | P | 70.1 | P |
| | | ug = Mm27494/len = 4804/STRA = for | | | | | | | |
| 9 | 92191_at | Cluster Incl AI255450 ui85f10 x1 Mus musculus cDNA, 3 end/clone = IMAGE-1889227/clone_end = 3/gb = AI255450/ | P | 109.1 | P | 83.8 | P | 84.3 | P |
| | | gi = 3862975/ug = Mm 34009/len = 509/STRA = for | | | | | | | |
| 9 | 95379_at | Cluster Incl AA245183 mw28g07 r1 Mus musculus cDNA, 5 end/clone = IMAGE-672060/clone_end = 5/gb = AA245183/ | P | 109.9 | P | 122.4 | P | 114.6 | P |
| | | gi = 1875900/ug = Mm 22988/len = 290/STRA = rev | | | | | | | |
| 9 | 99365_at | Cluster Incl AA734487 vt94c10 r1 Mus musculus cDNA, 5 end/clone = IMAGE-1178802/clone_end = 5/gb = AA734487/ | P | 312.4 | P | 386.3 | P | 296.2 | P |
| | | gi = 2756154/ug = Mm 5662/len = 513/STRA = rev | | | | | | | |
| 9 | 95498_at | Cluster Incl AI836029 UI-M-AQ0-aad-e-12-0-UI.s2 Mus musculus cDNA, 3 end/clone = UI-M-AQ0-aad-e-12-0-UI/ | P | 112 | P | 147.7 | P | 102.7 | P |
| | | clone_end = 3/gb = AI836029/gi = 5470207/ug = Mm 25143/len = 513/STRA = for | | | | | | | |
| 9 | 96670_at | Cluster Incl AI841295 UI-M-AM0-adu-g-05-0-UI s1 Mus musculus cDNA, 3 end/clone = UI-M-AM0-adu-g-05-0-UI/ | P | 139.7 | P | 242.9 | P | 131.3 | P |
| | | clone_end = 3/gb = AI841295/gi = 5475508/ug = Mm 27395/len = 441/STRA = for | | | | | | | |
| 9 | 99620_at | Cluster Incl AW060546 UI-M-BH1-ann-g-04-0-UI s1 Mus musculus cDNA, 3 end/clone = UI-M-BH1-ann-g-04-0-UI/ | A | 132.8 | P | 119.4 | P | 82.6 | P |
| | | clone_end = 3/gb = AW060546/gi = 6008297/ug = Mm 43213/len = 353/STRA = for | | | | | | | |
| 9 | 100612_at | Cluster Incl K02927 Ribonucleotide reductase M1/cds = (81,2459)/gb = K02927/gi = 200764/ug = Mm 656/len = 2902/ | P | 432.9 | P | 484.7 | P | 287.2 | A |
| | | STRA = for | | | | | | | |
| 9 | 103385_at | Cluster Incl U64033 Mus musculus Tera (Tera) mRNA, complete cds/cds = (81,914)/gb = U64033/gi = 1575504/ | P | 260.4 | P | 265.4 | A | 61.9 | A |
| | | ug = Mm.18637/len = 2720/STRA = for | | | | | | | |
| 9 | 95032_at | Cluster Incl AA856349: vw99g06 r1 Mus musculus cDNA, 5 end/clone = IMAGE-1263130/clone_end = 5/gb = AA856349/ | P | 367.4 | P | 295.7 | P | 110.1 | A |
| | | gi = 2944651/ug = Mm 27529/len = 291/STRA = for | | | | | | | |
| 9 | 95620_at | Cluster Incl AW120882 UI-M-BH2 3-aoa-h-10-0-UI.s1 Mus musculus cDNA, 3 end/clone = UI-M-BH2 3-aoa-h-10-0-UI/ | A | 200.8 | P | 202.7 | P | 73 | P |
| | | clone_end = 3/gb = AW120882/gi = 6096215/ug = Mm 2804/len = 327/STRA = for | | | | | | | |
| 9 | 97345_at | Cluster Incl AI849333 UI-M-AJ1-aha-e-03-0-UI.s1 Mus musculus cDNA, 3 end/clone = UI-M-AJ1-ahn-e-03-0-UI/ | P | 324.7 | P | 338.8 | P | 260.3 | P |
| | | clone_end = 3/gb = AI849333/gi = 5493239/ug = Mm.29613/len = 548/STRA = for | | | | | | | |
| 9 | 97421_at | Cluster Incl U42385: Fibroblast growth factor Inducible 16/cds = (45,1187)/gb = U42385/gi = 1353708/ug = Mm 2999/ | P | 407.8 | P | 226.5 | P | 104.7 | A |
| | | len = 2808/STRA = rev | | | | | | | |
| 9 | 100413_at | Cluster Incl AB033168 Mus musculus mRNA for nuclear protein ZAP, complete cds/cds = (101,4261)/gb = AB033168/ | P | 380.2 | P | 333.4 | P | 233.2 | A |
| | | gi = 6016841/ug = Mm 46674/len = 4698/STRA = for | | | | | | | |
| 9 | 101440_at | Cluster Incl AW047074 UI-M-BH1-alr-e-08-0-UI.s1 Mus musculus cDNA, 3 end/clone = UI-M-BH1-alr-e-08-0-UI/ | A | 210.8 | P | 208.1 | P | 202.8 | A |
| | | clone_end = 3/gb = AW047074/gi = 5907603/ug = Mm 778/len = 330/STRA = for | | | | | | | |
| 9 | 101835_at | Cluster Incl U10484 Mus musculus lymphoid-restricted membrane protein(Jaw1) mRNA, complete cds/cds = (151,1770)/ | P | 8.8 | P | −57 | A | −66.6 | A |
| | | gb = U10484/gi = 505683/ug = Mm 843/len = 2023/STRA = for | | | | | | | |
| 9 | 102646_at | Cluster Incl AW122284: UI-M-BH2 2-aos-b-02-0-UI s1 Mus musculus cDNA, 3 end/clone = UI-M-BH2-2-aos-b-02-0-UI/ | P | 158.3 | P | 147.4 | P | 102.5 | P |
| | | clone_end = 3/gb = AW122284/gi = 6097765/ug = Mm 13148/len = 336/STRA = for | | | | | | | |
| 9 | 103939_at | Cluster Incl AW121399 UI-M-BH2 2-aom-h-02-0-UI s1 Mus musculus cDNA, 3 end/clone = UI-M-BH2.2-aom-h-02-0-UI/ | P | 147.6 | P | 97 | P | 70.9 | P |
| | | clone_end = 3/gb = AW121399/gi = 6096732/ug = Mm 25250/len = 550/STRA = for | | | | | | | |
| 9 | 104247_at | Cluster Incl AI846291: UI-M-AK1-aez-f-04-0-UI.s1 Mus musculus cDNA, 3 end/clone = UI-M-AK1-aez-f-04-0-UI/ | P | 205.8 | P | 209.9 | P | 178.4 | A |
| | | clone_end = 3/gb = AI846291/gi = 5490197/ug = Mm.28335/len = 287/STRA = for | | | | | | | |
| 9 | 104431_at | Cluster Incl D11091 Protein Kinase C, theta/cds = (88,2211)/gb = D11091/gi = 220573/ug = Mm.2921/len = 3313/STRA = for | P | 185.8 | P | 181.6 | P | 171.2 | A |
| 9 | 92468_at | Cluster Incl AF028722 Mus musculus fetal globin inducing factor mRNA, complete cds/cds = (135,851)/gb = AF028722/ | P | 182.1 | P | 203.1 | P | 97.9 | M |
| | | gi = 4103856/ug = Mm.42088/len = 1801/STRA = for | | | | | | | |
| 9 | 98335_at | Cluster Incl X72711 Replication factor C, 140 KDa/cds = (79,3474)/gb = X72711/gi = 457741/ug = Mm 4280/len = 4679/ | P | 250.5 | P | 238 | P | 151.7 | A |
| | | STRA = for | | | | | | | |
| 9 | 92788_f_at | Cluster Incl Y12474 M musculus mRNA for centrin gene/cds = (0,503)/gb = Y12474/gi = 2246423/ug = Mm.12481/len = 504/ | P | 942.6 | P | 685.4 | P | 576.6 | A |
| | | STRA = for | | | | | | | |
| 9 | 98146_at | Cluster Incl AW120775: UI-M-BH2.3-any-f-10-0-UI.s1 Mus musculus cDNA, 3 end/clone = UI-M-BH2.3-any-f-10-0-UI/ | A | 317.7 | P | 222 | P | 246.5 | P |
| | | clone_end = 3/gb = AW120775/gi = 6096108/ug = Mm.35698/len = 463/STRA = for | | | | | | | |
| 9 | 103354_at | Cluster Incl Z46966: M. musculus mRNA for imogen 44/cds = (45,1199)/gb = Z46966/gi = 1272668/ug = Mm.1841/len = 1267/ | P | 293.7 | P | 284.5 | P | 246.7 | A |
| | | STRA = for | | | | | | | |
| 9 | 95586_at | Cluster Incl AF089751 Mus musculus ATP receptor P2X4 subunit mRNA, complete cds/cds = (31,1197)/gb = AF089751/ | P | 170.2 | P | 131.6 | P | 142.3 | P |
| | | gi = 4019303/ug = Mm 27861/len = 1944/STRA = for | | | | | | | |
| 9 | 98409_at | Cluster Incl L00923 Mouse myosin I mRNA, complete cds/cds = (196,3435)/gb = L00923/gi = 200008/ug = Mm 3390/ | P | 272.9 | P | 203.3 | P | 141.1 | A |
| | | len = 4194/STRA = for | | | | | | | |
| 9 | 98995_at | Cluster Incl X86925 M musculus mRNA for E2F-5 protein/cds = (15,1022)/gb = X86925/gi = 806571/ug = Mm.379/len = 1340/ | P | 99.2 | P | 85.2 | P | 85.3 | A |
| | | STRA = for | | | | | | | |
| 9 | 99937_at | Cluster Incl Z16406 Mesenchyme homeobox 2/cds = (163,1074)/gb = Z16406/gi = 57949/ug = Mm 4346/len = 1440/ | P | 126.8 | P | 124.9 | P | 85.2 | P |
| | | STRA = for | | | | | | | |
| 9 | 100014_at | Cluster Incl AI845038 UI-M-BG0-ahy-b-01-0-UI.s1 Mus musculus cDNA, 3 end/clone = UI-M-BG0-ahy-b-01-0-UI/ | P | 86.1 | P | 97.8 | P | 44.8 | A |
| | | clone_end = 3/gb = AI845038/gi = 5488944/ug = Mm 4557/len = 380/STRA = for | | | | | | | |
| 9 | 101444_at | Cluster Incl U83176 Gene trap ROSA 26 antisense/cds = (90,1607)/gb = U83176/gi = 1778860/ug = Mm 781/len = 1974/ | P | 246.9 | P | 233.6 | P | 189.7 | P |
| | | STRA = for | | | | | | | |
| 9 | 93436_i_at | Cluster Incl AI850509 UI-M-BG1-aih-h-10-0-UI s1 Mus musculus cDNA, 3 end/clone = UI-M-BG1-aih-h-10-0-UI/ | P | 67.3 | P | 77.6 | P | 67.7 | P |
| | | clone_end = 3/gb = AI850509/gi = 5494415/ug = Mm 65832/len = 453/STRA = for | | | | | | | |
| 9 | 93470_at | Cluster Incl AF012129 Mus musculus putative DNA methyltransferase (Dnmt2) mRNA, complete cds/cds = (19,1266)/ | P | 67.5 | P | 60.1 | P | 68.4 | A |
| | | gb = AF012129/gi = 2636723/ug = Mm 6979/len = 1691/STRA = for | | | | | | | |
| 9 | 100138_f_at | Cluster Incl X52102 M musculus p16K gene for 16 kDa protein/cds = (77,1798)/gb = X52102/gi = 53546/ug = Mm 4877/ | P | 237.2 | P | 353.6 | P | 138.4 | A |
| | | len = 2751/STRA = for | | | | | | | |
| 9 | 100148_at | Cluster Incl U51037 Mus musculus 11-zinc-finger transcription factor (CTCF) mRNA, complete cds/cds = (306,2516)/ | P | 271.7 | P | 149.1 | P | 104.5 | A |
| | | gb = U51037/gi = 1256409/ug = Mm 493/len = 3840/STRA = for | | | | | | | |
| 9 | 103081_at | Cluster Incl AA863928 vx15h09 r1 Mus musculus cDNA, 5 end/clone = IMAGE-1264577/clone_end = 5/gb = AA863928/ | P | 426.8 | P | 459.6 | P | 416.9 | M |
| | | gi = 2956407/ug = Mm 1717/len = 273/STRA = for | | | | | | | |
| 9 | 95561_at | Cluster Incl AW120867 UI-M-BH2 3-aoa-g-06-0-UI s1 Mus musculus cDNA, 3 end/clone = UI-M-BH2 3-aoa-g-06-0-UI/ | P | 136.1 | P | 146.2 | P | 57.3 | A |
| | | clone_end = 3/gb = AW120867/gi = 6096200/ug = Mm 27777/len = 312/STRA = for | | | | | | | |
| 9 | 98515_at | Cluster Incl AI844392 UI-M-AL1-ahp-d-12-0-UI s1 Mus musculus cDNA, 3 end/clone = UI-M-AL1-ahp-d-12-0-UI/ | P | 362.3 | A | 382.9 | P | 13.9 | A |
| | | clone_end = 3/gb = AI844392/gi = 5488298/ug = Mm 36088/len = 445/STRA = for | | | | | | | |
| 9 | 98979_at | Cluster Incl U35249 Mus musculus CDK-activating kinase assembly factor p36/MAT1, complete cds/cds = (152,1081)/ | P | 80.2 | P | 76.8 | A | 84.3 | P |
| | | gb = U35249/gi = 1015998/ug = Mm 3688/len = 1409/STRA = for | | | | | | | |
| 9 | 100955_at | Cluster Incl AA989957: ua55a07 r1 Mus musculus cDNA, 5 end/clone = IMAGE-1361364/clone_end = 5/gb = AA989957/ | A | 110.5 | P | 87.7 | P | 87 | P |
| | | gi = 3175321/ug = Mm 6476/len = 456/STRA = for | | | | | | | |
| 9 | 102194_at | Cluster Incl AW122332 UI-M-BH2 2-aoa-f-10-0-UI s1 Mus musculus cDNA, 3 end/clone = UI-M-BH2 2-aoa-f-10-0-UI/ | P | 195.1 | P | 293.8 | P | 129.5 | M |
| | | clone_end = 3/gb = AW122332/gi = 6097817/ug = Mm 98631/len = 303/STRA = rev | | | | | | | |
| 9 | 102293_at | Cluster Incl L03547 Early lymphoid specific transcription factor/cds = (38,1333)/gb = L03547/gi = 198286/ug = Mm 12396/ | P | 92.8 | P | 79.6 | A | 65.4 | A |
| | | len = 1550/STRA = rev | | | | | | | |
| 9 | 104451_at | Cluster Incl AI852578 UI-M-BH0-aiu-a-08-0-UI s1 Mus musculus cDNA, 3 end/clone = UI-M-BH0-aiu-a-08-0-UI/ | P | 282.3 | P | 294.1 | P | 235 | A |
| | | clone_end = 3/gb = AI852578/gi = 5496484/ug = Mm.29433/len = 417/STRA = for | | | | | | | |
| 9 | 93217_at | Cluster Incl AW049642 UI-M-BH1-and-g-02-0-UI s1 Mus musculus cDNA, 3 end/clone = UI-M-BH1-and-g-02-0-UI/ | A | 346.8 | P | 337.8 | P | 102.1 | A |
| | | clone_end = 3/gb = AW049642/gi = 5910171/ug = Mm 5710/len = 444/STRA = for | | | | | | | |
| 9 | 93560_at | Cluster Incl AI845882 UI-M-AO1-aeg-h-05-0-UI s1 Mus musculus cDNA, 3 end/clone = UI-M-AO1-aeg-h-05-0-UI/ | P | 241.5 | P | 277.8 | A | 212.5 | M |
| | | clone_end = 3/gb = AI845882/gi = 5489788/ug = Mm.20309/len = 423/STRA = for | | | | | | | |
| 9 | 94000_at | Cluster Incl X07698: Mouse mRNA for CD8 molecule Ly-3 chain/cds = (49,690)/gb = X07698/gi = 52954/ug = Mm 21764/ | A | 306.9 | P | 374.8 | P | 313.8 | P |
| | | len = 1111/STRA = for | | | | | | | |
| 9 | 96656_at | Cluster Incl AI644179: vw55g04.x1 Mus musculus cDNA, 3 end/clone = IMAGE-1247766/clone_end = 3/gb = AI644179/ | A | 134.7 | P | 114.6 | P | 87.4 | A |
| | | gi = 4722654/ug = Mm 27339/len = 269/STRA = for | | | | | | | |
| 9 | 99129_at | Cluster Incl AW124529 UI-M-BH2.1-apo-g-08-0-UI s1 Mus musculus cDNA, 3 end/clone = UI-M-BH2 1-apo-g-08-0-UI/ | P | 173.3 | P | 90.2 | A | 27.7 | A |
| | | clone_end = 3/gb = AW124529/gi = 6100059/ug = Mm 41043/len = 503/STRA = for | | | | | | | |
| 9 | 101108_at | Cluster Incl AF034610 Mus musculus nuclear autoantigenic sperm protein mRNA, complete cds/cds = (91,2412)/ | P | 194 | P | 72 | P | 22.6 | A |
| | | gb = AF034610/gi = 2645972/ug = Mm 7516/len = 2543/STRA = for | | | | | | | |
| 9 | 104296_at | Cluster Incl AI847802: UI-M-AK1-aev-g-02-0-UI s1 Mus musculus cDNA, 3 end/clone = UI-M-AK1-aev-g-02-0-UI/ | M | 187.9 | P | 189.5 | P | 103.7 | A |
| | | clone_end = 3/gb = AI847802/gi = 5491708/ug = Mm 22334/len = 520/STRA = for | | | | | | | |
| 9 | 104535_at | Cluster Incl AF090430 Mus musculus ATP-dependent metalloprotease FlsH1 mRNA, complete cds/cds = (172,2319)/ | A | 82.4 | P | 44.7 | A | 34.8 | A |
| | | gb = AF090430/gi = 3600099/ug = Mm 23335/len = 2654/STRA = for | | | | | | | |
| 9 | 96216_at | Cluster Incl AI848826 UI-M-AJ1-ahb-b-04-0-UI s1 Mus musculus cDNA, 3 end/clone = UI-M-AJ1-ahb-b-04-0-UI/ | P | 163.6 | P | 138.5 | P | 167.6 | A |
| | | clone_end = 3/gb = AI848826/gi = 5492732/ug = Mm 28677/len = 493/STRA = for | | | | | | | |
| 9 | 102625_f_at | Cluster Incl AF080070: Mus musculus zinc finger protein 54 (Zfp54) mRNA, complete cds/cds = (121,2115)/gb = AF080070/ | P | 49 | P | 65.6 | P | 19.7 | A |
| | | gi = 3406675/ug = Mm 12916/len = 2267/STRA = rev | | | | | | | |
| 9 | 93529_at | Cluster Incl AW125219 UI-M-BH2 1-aps-c-07-0-UI.s1 Mus musculus cDNA, 3 end/clone = UI-M-BH2 1-aps-c-07-0-UI/ | P | 184.6 | P | 194.8 | P | 110.2 | M |
| | | clone_end = 3/gb = AW125219/gi = 6100749/ug = Mm 19803/len = 345/STRA = for | | | | | | | |
| 9 | 93758_at | Cluster Incl AA823653: vr69c07.s1 Mus musculus cDNA, 5 end/clone = IMAGE-1125900/clone_end = 5/gb = AA823653/ | P | 212.7 | P | 208.7 | P | 102.6 | A |
| | | gi = 2893521/ug = Mm.21140/len = 554/STRA = for | | | | | | | |
| 9 | 94509_at | Cluster Incl AI851207 UI-M-BH0-ajx-f-06-0-UI s1 Mus musculus cDNA, 3 end/clone = UI-M-BH0-ajx-f-06-0-UI/clone_end = 3/ | P | 238.6 | P | 174.2 | A | 87.3 | A |
| | | gb = AI851207/gi = 5495113/ug = Mm 28968/len = 332/STRA = for | | | | | | | |
| 9 | 96902_at | Cluster Incl AW121847 UI-M-BH2 3-aoi-a-12-0-UI.s1 Mus musculus cDNA, 3 end/clone = UI-M-BH2 3-aoi-a-12-0-UI/ | P | 223.9 | P | 242.1 | M | 202.9 | A |
| | | clone_end = 3/gb = AW121847/gi = 6097221/ug = Mm 3012/len = 241/STRA = for | | | | | | | |
| 9 | 96924_at | Cluster Incl AI849719 UI-M-AL1-ahn-b-05-0-UI.s1 Mus musculus cDNA, 3 end/clone = UI-M-AL1-ahn-b-05-0-UI/ | A | 504.5 | P | 534.3 | P | 325.5 | P |
| | | clone_end = 3/gb = AI849719/gi = 5493625/ug = Mm 30160/len = 413/STRA = for | | | | | | | |
| 9 | 99185_at | Cluster Incl AW047026 UI-M-BH1-alr-a-02-0-UI s1 Mus musculus cDNA, 3 end/clone = UI-M-BH1-alr-a-02-0-UI/ | P | 204.2 | P | 163.5 | A | 133.3 | M |
| | | clone_end = 3/gb = AW047026/gi = 5907555/ug = Mm 41874/len = 257/STRA = for | | | | | | | |
| 9 | 100577_at | Cluster Incl M58558: Small nuclear ribonucleoprotein D1/cds = (46,405)/gb = M58558/gi = 200993/ug = Mm.603/len = 506/ | P | 747.1 | A | 741.2 | P | 313.2 | A |
| | | STRA = for | | | | | | | |
| 9 | 104097_at | Cluster Incl AF002823 Budding inhibited by benzimidazoles 1 (S. cerevislae) homolog/cds = (60,3236)/gb = AF002823/ | M | 113.2 | P | 54 | P | −60.4 | A |
| | | gi = 2150135/ug = Mm 2185/len = 3370/STRA = for | | | | | | | |
| 9 | 95544_at | Clustor Incl AI843150 UI-M-AK1-aes-h-12-0-UI s1 Mus musculus cDNA, 3 end/clone = UI-M-AK1-aes-h-12-0-UI/ | P | 195.8 | P | 215.8 | P | 21.2 | M |
| | | clone_end = 3/gb = AI843150/gi = 5477363/ug = Mm 27687/len = 229/STRA = for | | | | | | | |
| 9 | 99541_at | Cluster Incl AJ223293 Mus musculus mRNA for kinesin-related mitotic motor protein/cds = (0,3044)/gb = AJ223293/ | A | 112 | P | 192.8 | P | 9.1 | A |
| | | gi = 4160555/ug = Mm.42203/len = 4412/STRA = for | | | | | | | |
| 9 | 102303_f_at | Cluster Incl L28167: Zinc finger protein 61/cds = (927,2594)/gb = L28167/gi = 758660/ug = Mm.12832/len = 2606/STRA = for | M | 275.9 | P | 268.7 | M | 226.6 | P |
| 9 | 103685_at | Cluster Incl AI839232 UI-M-AKO-adh-d-11-0-UI.s1 Mus musculus cDNA, 3 end/clone = UI-M-AKO-adh-d-11-0-UI/ | A | 90.3 | P | 62.7 | P | 16.2 | A |
| | | clone_end = 3/gb = AI839232/gi = 5473445/ug = Mm 23897/len = 407/STRA = for | | | | | | | |
| 9 | 93228_at | Cluster Incl U25691: Helicase, lymphoid specific/cds = (39,1850)/gb = U25691/gi = 805295/ug = Mm.57223/len = 1851/ | P | 102.6 | P | 81.5 | P | 48.7 | A |
| | | STRA = for | | | | | | | |
| 9 | 100306_at | Cluster Incl AI510297 vx94c04 y1 Mus musculus cDNA, 5 end/clone = IMAGE-1293606/clone_end = 5/gb = AI510297/ | P | 133.4 | P | 99.9 | A | 87.5 | A |
| | | gi = 4409202/ug = Mm 86721/len = 434/STRA = for | | | | | | | |
| 10 | 93055_at | Cluster Incl AW049376 UI-M-BH1-ane-c-05-0-UI s1 Mus musculus cDNA, 3 end/clone = UI-M-BH1-ane-c-05-0-UI/ | P | 166.6 | P | 130.4 | P | 179.9 | P |
| | | clone_end = 3/gb = AW049376/gi = 5909905/ug = Mm 15351/len = 492/STRA = for | | | | | | | |
| 10 | 93258_at | Cluster Incl M28666 Hydroxymethylbilane synthase/cds = (0,1202)/gb = M28666/gi = 200231/ug = Mm 1710/len = 1203/ | P | 186 | P | 184.1 | P | 195.6 | P |
| | | STRA = for | | | | | | | |
| 10 | 93496_at | Cluster Incl AI852098 UI-M-BH0-aja-h-11-0-UI s1 Mus musculus cDNA, 3 end/clone = UI-M-BH0-aja-h-11-0-UI/ | P | 838.5 | P | 818.9 | P | 711.7 | P |
| | | clone_end = 3/gb = AI852098/gi = 5496004/ug= Mm 19130/len= 504/STRA = for | | | | | | | |
| 10 | 93521_at | Cluster Incl AW049194 UI-M-BH1-amq-g-12-0-UI.s1 Mus musculus cDNA, 3 end/clone = UI-M-BH1-amq-g-12-0-UI/ | P | 804.9 | P | 721.1 | P | 609.2 | P |
| | | clone_end = 3/gb = AW049194/gi = 5909723/ug = Mm 1963/len = 272/STRA = for | | | | | | | |
| 10 | 93782_at | Cluster Incl AI844517: UI-M-AJ1-ahf-a-02-0-UI s2 Mus musculus cDNA, 3 end/clone = UI-M-AJ1-ahf-a-02-0-UI/ | P | 1093.3 | P | 928.9 | P | 994.8 | P |
| | | clone_end = 3/gb = AI844517/gi = 5488423/ug = Mm 21281/len = 504/STRA = for | | | | | | | |
| 10 | 93831_at | Cluster Incl AI316087: uk62h02 y1 Mus musculus cDNA, 5 end/clone = IMAGE-1973619/clone_end = 5/gb = AI316087/ | P | 271.1 | P | 246.8 | P | 275.1 | P |
| | | gi = 4031354/ug = Mm 21559/len = 512/STRA = for | | | | | | | |
| 10 | 94007_at | Cluster Incl AW122469 UI-M-BH2 2-aou-d-04-0-UI s1 Mus musculus cDNA, 3 end/clone = UI-M-BH2 2-aou-d-04-0-UI/ | P | 3128 | P | 2746.7 | P | 2420.3 | P |
| | | clone_end = 3/gb = AW122469/gi = 6097989/ug = Mm 21802/len = 224/STRA = for | | | | | | | |
| 10 | 94225_at | Cluster Incl AI844679 UI-M-AL1-ahr-f-03-0-UI s1 Mus musculus cDNA, 3 end/clone = UI-M-AL1-ahr-f-03-0-UI/clone_end = 3/ | P | 458 | P | 465.1 | P | 456.1 | P |
| | | gb = AI844679/gi = 5488585/ug = Mm.22264/len = 475/STRA = for | | | | | | | |
| 10 | 94257_at | Cluster Incl AI836117 UI-M-AQ0-aae-c-02-0-UI s2 Mus musculus cDNA, 3 end/clone = UI-M-AQ0-aae-c-02-0-UI/ | P | 225.6 | P | 155.2 | P | 251.5 | P |
| | | clone_end = 3/gb = AI836117/gi = 5470325/ug = Mm 2241/len = 289/STRA = for | | | | | | | |
| 10 | 94286_at | Cluster Incl AW050340: UI-M-BH1-ang-c-08-0-UI s1 Mus musculus cDNA, 3 end/clone = UI-M-BH1-ang-c-08-0-UI/ | P | 304.6 | P | 323.5 | P | 440.1 | P |
| | | clone_end = 3/gb = AW050340/gi = 5910869/ug = Mm 22565/len = 492/STRA = for | | | | | | | |
| 10 | 94853_at | Cluster Incl U29055 Guanine nuleotide binding protein, beta 1/cds = (39,1061)/gb = 32 U29055/gi = 868166/ug = Mm 2344/ | P | 587.6 | P | 625.8 | P | 581.5 | P |
| | | len = 1410/STRA = for | | | | | | | |
| 10 | 94854_g_at | Cluster Incl U29055 Guanine nucleotide binding protein, beta 1/cds = (39,1061)/gb = U29055/gi = 868166/ug = Mm 2344/ | P | 547.8 | P | 607.9 | P | 707.5 | P |
| | | len = 1410/STRA = for | | | | | | | |
| 10 | 94860_at | Cluster Incl AW121821 UI-M-BH2 3-anx-d-12-0-UI s2 Mus musculus cDNA, 3 end/clone = UI-M-BH2 3-anx-d-12-0-UI/ | P | 730.8 | P | 747 | P | 623.7 | P |
| | | clone_end = 3/gb = AW121821/gi = 6097189/ug = Mm 2368/len = 370/STRA = for | | | | | | | |
| 10 | 94871_r_at | Cluster Incl AW124226: UI-M-BH2 1-aph-d-10-0-UI.s1 Mus musculus cDNA, 3 end/clone = UI-M-BH2 1-aph-d-10-0-UI/ | P | 708.2 | P | 532.2 | P | 666.8 | P |
| | | clone_end = 3/gb = AW124226/gi = 6099756/ug = Mm 23755/len = 382/STRA = for | | | | | | | |
| 10 | 94895_at | Cluster Incl AW122948: UI-M-BH2. 1-aoz-c-05-0-UI s2 Mus musculus cDNA, 3 end/clone = UI-M-BH2.1-aoz-c-05-0-UI/ | P | 431.5 | P | 487.9 | P | 375.8 | P |
| | | clone_end = 3/gb = AW122948/gi = 6098478/ug = Mm 23998/len = 451/STRA = for | | | | | | | |
| 10 | 94896_at | Cluster Incl D90151 Mouse mRNA for CArG-binding factor-A, complete cds/cds = (193,1050)/gb = D90151/gi = 220362/ | P | 715 | P | 845.6 | P | 691.3 | P |
| | | ug = Mm.24/len = 1595/STRA = for | | | | | | | |
| 10 | 94910_at | Cluster Incl AW120739: UI-M-BH2.3-any-c-04-0-UI.s1 Mus musculus cDNA, 3 end/clone = UI-M-BH2.3-any-c-04-0-UI/ | P | 189 | P | 157.1 | P | 106.8 | P |
| | | clone_end = 3/gb = AW120739/gi = 6096072/ug = Mm 24105/len = 445/STRA = for | | | | | | | |
| 10 | 95419_at | Cluster Incl M29260 Mouse histone 1-0 gene, 5 end promoter region/cds = (266,268)/gp = M29260/gi = 193849/ | P | 1937.6 | P | 2207.1 | P | 2097.1 | P |
| | | ug = Mm 24350/len = 269/STRA = for | | | | | | | |
| 10 | 95460_at | Clustor Incl U70736 Mus musculus 38 kDa Mov34 homolog mRNA, complete cds/cds = (123,1127)/gb = U70736/ | P | 559.1 | P | 524.6 | P | 537.4 | P |
| | | gi = 2360946/ug = Mm 2472/len = 1190/STRA = for | | | | | | | |
| 10 | 96065_at | Cluster Incl D88769 Mus musculus mRNA fo latexin, complete cds/cds = (99,767)/gp = U88769/gi = 1669620/ug = Mm 2632 | P | 320.8 | P | 192 | P | 210.8 | P |
| | | len = 996/STRA = for | | | | | | | |
| 10 | 96606_at | Cluster Incl AB025217: Mus musculus mRNA for Sid470p, complete cds/cds = (55, 642)/gb = AB025217/gi = 5931554/ | P | 274.6 | P | 242.1 | P | 358.9 | P |
| | | ug = Mm 27065/len = 782/STRA = for | | | | | | | |
| 10 | 96626_at | Cluster Incl AI843401 UI-M-AQ1-adz-h-01-0-UI.s1 Mus musculus cDNA, 3 end/clone = UI-M-AQ1-adz-h-01-0-UI/ | P | 514.5 | P | 448.8 | P | 575 | P |
| | | clone_end = 3/gb = AI843401/gi = 5477614/ug = Mm 27182/len = 336/STRA = for | | | | | | | |
| 10 | 96700_r_at | Cluster Incl AW060594 UI-M-BH1-anq-d-05-0-UI.s1 Mus musculus cDNA, 3 end/clone = UI-M-BH1-anq-d-05-0-UI/ | P | 80.1 | P | 62.1 | P | 53.8 | P |
| | | clone_end = 3/gb = AW060594/gi = 6008345/ug = Mm 27560/len = 245/STRA = for | | | | | | | |
| 10 | 97272_at | Cluster Incl M99167 Mouse rearranged RNA binding protein (FLI-2) mRNA, complete cds/cds = (59,1021)/gb = M99167/ | P | 660.3 | P | 711.3 | P | 675.2 | P |
| | | gi = 193323/ug = Mm 27927/len = 1443/STRA = for | | | | | | | |
| 10 | 97312_at | Cluster Incl AB014464 Mus musculus mRNA for MGC-24v, complete cds/cds = (0,593)/gb = AB014464/gi = 3892655/ | P | 2038 | P | 1311.7 | P | 1805.8 | P |
| | | ug = Mm 28083/len = 594/STRA = for | | | | | | | |
| 10 | 97818_at | Cluster Incl AI838691 UI-M-AO0-aca-g-07-0-UI.s1 Mus musculus cDNA, 3 end/clone = UI-M-AO0-aca-g-07-0-UI/ | P | 757 | P | 543.9 | P | 622.7 | P |
| | | clone_end = 3/gb = AI838691/gi = 5472904/ug = Mm 28196/len = 387/STRA = for | | | | | | | |
| 10 | 97843_at | Cluster Incl AI834866: UI-M-AN1-afe-a-11-0-UI s1 Mus musculus cDNA, 3 end/clone = UI-M-AN1-afe-a-11-0-UI/ | P | 907.3 | P | 514.8 | P | 583.8 | P |
| | | clone_end = 3/gb = AI834866/gi = 5469079/ug = Mm 28261/len = 488/STRA = for | | | | | | | |
| 10 | 98917_at | Clustor Incl AA823202: vw41h10r1 Mus musculus cDNA, 5 end/clone = IMAGE-1246435/clone_end = 5/gb = AA823202/ | P | 588.5 | P | 478 | P | 573.7 | P |
| | | gi = 2893070/ug = Mm 2862/len = 363/STRA = for | | | | | | | |
| 10 | 98929_at | Cluster Incl AI846452: UI-M-AQ1-aee-f-09-0-UI.s1 Mus musculus cDNA, 3 end/clone = UI-M-AQ1-aee-f-09-0-UI/ | P | 399.9 | P | 377.8 | P | 348.6 | P |
| | | clone_end = 3/gb = AI846452/gi = 5490358/ug = Mm.28664/len = 283/STRA = for | | | | | | | |
| 10 | 94562_at | Cluster Incl AI843968: UI-M-AP1-agh-e-10-0-UI s1 Mus musculus cDNA, 3 end/clone = UI-M-AP1-agh-e-10-0-UI/ | P | 508.1 | P | 479.3 | P | 600 | P |
| | | clone_end = 3/gb = AI843968/gi = 5478181/ug = Mm 29114/len = 334/STRA = for | | | | | | | |
| 10 | 95153_at | Cluster Incl AI845988: UI-M-AP1-agl-d-01-0-UI s1 Mus musculus cDNA, 3 end/clone = UI-M-AP1-agl-d-01-0-UI/ | P | 155.7 | P | 156.6 | P | 312.2 | P |
| | | clone_end = 3/gb = AI845988/gi = 5489894/ug = Mm 29476/len = 435/STRA = for | | | | | | | |
| 10 | 95650_at | Cluster Incl U95114 Mus musculus fertilization antigen-1 mRNA, complete cds/cds = (82,576)/gb = U95114/gi = 2098784/ | P | 1049.8 | P | 980.2 | P | 915.3 | P |
| | | ug = Mm.29504/len = 642/STRA = for | | | | | | | |
| 10 | 95665_at | Cluster Incl AI852087 UI-M-BH0-aja-g-04-0-UI s1 Mus musculus cDNA, 3 end/clone = UI-M-BH0-aja-g-04-0-UI/ | P | 332.3 | P | 162.9 | P | 229.4 | P |
| | | clone_end = 3/gb = AI852087/gi = 5495993/ug = Mm 29571/len = 260/STRA = for | | | | | | | |
| 10 | 95754_at | Cluster Incl AI838216 UI-M-AL0-abx-c-12-0-UI s1 Mus musculus cDNA, 3 end/clone = UI-M-AL0-abx-c-12-0-UI/ | P | 694.8 | P | 585.5 | P | 273.2 | P |
| | | clone_end = 3/gb = AI838216/gi = 5472429/ug = Mm 29791/len = 474/STRA = for | | | | | | | |
| 10 | 96243_f_at | Cluster Incl AW120804 UI-M-BH2 3-aoa-a-08-0-UI s1 Mus musculus cDNA, 3 end/clone = UI-M-BH2 3-aoa-a-08-0-UI/ | P | 317 | P | 236.4 | P | 228.3 | P |
| | | clone_end = 3/gb = AW120804/gi = 6096137/ug = Mm 29803/len = 289/STRA = for | | | | | | | |
| 10 | 96257_at | Cluster Incl AW124376 UI-M-BH2 1-ape-a-11-0-UI s1 Mus musculus cDNA, 3 end/clone = UI-M-BH2 1-ape-a-11-0-UI/ | P | 657.5 | P | 550.8 | P | 616.9 | P |
| | | clone_end = 3/gb = AW124376/gi = 6099906/ug = Mm 29822/len = 213/STRA = for | | | | | | | |
| 10 | 97488_at | Cluster Incl AI595893: uk19g08 x1 Mus musculus cDNA, 3 end/clone = IMAGE-1969502/clone_end = 3/gb = AI595893/ | P | 441.7 | P | 652.6 | P | 503.4 | P |
| | | gi = 4604941/ug = Mm 3094/len = 448/STRA = for | | | | | | | |
| 10 | 98586_at | Cluster Incl AW107230: um12f03 x1 Mus musculus cDNA, 3 end/clone = IMAGE-2182877/clone_end = 3/gb = AW107280/ | P | 355.1 | P | 246.4 | P | 214.4 | P |
| | | gi = 6078030/ug = Mm.3797/len = 574/STRA = for | | | | | | | |
| 10 | 99162_at | Cluster Incl AW122398: UI-M-BH2 2-aow-d-11-0-UI s1 Mus musculus cDNA, 3 end/clone = UI-M-BH2 2-aow-d-11-0-UI/ | P | 238 | P | 169.8 | P | 219.1 | P |
| | | clone_end = 3/gb = AW122398/gi = 6097899/ug = Mm 41670/len = 506/STRA = for | | | | | | | |
| 10 | 99575_at | Cluster Incl AA517835 vh82b01 r1 Mus musculus cDNA, 3 end/clone = IMAGE-893449/clone_end = 3/gb = AA517835/ | P | 1578.4 | P | 1443.2 | P | 1601.1 | P |
| | | gi = 2257359/ug = Mm 4230/len = 440/STRA = for | | | | | | | |
| 10 | 100073_at | Cluster Incl AW045965 UI-M-BH1-aku-h-09-0-UI s1 Mus musculus cDNA, 3 end/clone = UI-M-BH1-aku-h-09-0-UI/ | P | 187 | P | 198.9 | P | 181.7 | P |
| | | clone_end = 3/gb = AW045965/gi = 5906494/ug = Mm 45225/len = 554/STRA = for | | | | | | | |
| 10 | 101096_s_at | Cluster Incl AF023482 Mus musculus HS1-associating protein (mHAX-1s) mRNA, complete cds/cds = (108,950)/ | P | 388.7 | P | 353.4 | P | 339.8 | P |
| | | gb = AF023482/gi = 2522493/ug = Mm 7401/len = 1121/STRA = for | | | | | | | |
| 10 | 101101_at | Cluster Incl Z67746 M musculus mRNA for phosphatase 2A catalytic subnit, isotype beta/cds = (0,929)/gb = Z67746/ | P | 604.5 | P | 586.7 | P | 634.3 | P |
| | | gi = 1246796/ug = Mm 7418/len = 930/STRA = for | | | | | | | |
| 10 | 101960_at | Cluster Incl AI842208 UI-M-AI1-afm-f-10-0-UI s1 Mus musculus cDNA, 3 end/clone = UI-M-AI1-afm-f-10-0-UI/clone_end = 3/ | P | 423.5 | P | 361.6 | P | 458.8 | P |
| | | gb = AI842208/gi = 5476421/ug = Mm 9257/len = 382/STRA = for | | | | | | | |
| 10 | 102019_at | Cluster Incl AA666635 vm50b08 r1 Mus musculus cDNA, 5 end/clone = IMAGE-1001655/clone_end = 5/gb = AA666835/ | P | 451 | P | 479.2 | P | 435.8 | P |
| | | gi = 2625336/ug = Mm 10076/len = 203/STRA = for | | | | | | | |
| 10 | 102039_at | Cluster Incl AI850881 UI-M-BH0-ajt-f-06-UI s1 Mus musculus cDNA, 3 end/clone = UI-M-BH0-ajt-1-06-0-UI/clone_end = 3/ | P | 289.9 | P | 286.2 | P | 309.7 | P |
| | | gb = AI850881/gi = 5494787/ug = Mm 10182/len = 471/STRA = for | | | | | | | |
| 10 | 103047_at | Cluster Incl AF031128 Mus musculus peroxisome membrane protein PEX2 mRNA, complete cds/cds = (501,1418)/ | P | 303.5 | P | 282.8 | P | 285.8 | P |
| | | gb = AF031128/gi = 2623263/ug = Mm 16453/len = 1749/STRA = for | | | | | | | |
| 10 | 103428_at | Cluster Incl AW049684 UI-M-BH1-ani-b-12-0-UI s1 Mus musculus cDNA, 3 end/clone = UI-M-BH1-ani-b-12-0-UI/ | P | 270.9 | P | 251.6 | P | 272 | P |
| | | clone_end = 3/gb = AW049684/gi = 5910213/ug = Mm.19000/len = 346/STRA = for | | | | | | | |
| 10 | 103848_at | Cluster Incl AW121188 UI-M-BH2.3-aok-b-04-0-UI.s1 Mus musculus cDNA, 3 end/clone = UI-M-BH2.3-aok-b-04-0-UI/ | P | 360.8 | P | 375.5 | P | 348.6 | P |
| | | clone_end = 3/gb = AW121188/gi = 6096521/ug = Mm.21048/len = 477/STRA = for | | | | | | | |
| 10 | 103861_s_at | Cluster Incl AA388099: vc88g07.r1 Mus musculus cDNA, 5 end/clone = IMAGE-790140/clone_end = 5/gb = AA388099/ | P | 222.1 | P | 312.8 | P | 256.5 | P |
| | | gi = 2041044/ug = Mm.21103/len = 564/STRA = for | | | | | | | |
| 10 | 104297_at | Cluster Incl AW124742 UI-M-BH2 1-aqa-d-07-0-UI s1 Mus musculus cDNA, 3 end/clone = UI-M-BH2 1-aqa-d-07-0-UI/ | P | 306.8 | P | 349.5 | P | 295.8 | P |
| | | clone_end = 3/gb = AW124742/gi = 6100272/ug = Mm.22336/len = 466/STRA = for | | | | | | | |
| 10 | 94360_at | Cluster Incl AA672650 vp57b04 r1 Mus musculus cDNA, 3 end/clone = IMAGE-1080751/clone_end = 3/gb = AA672650/ | P | 265.8 | P | 192.9 | P | 343.6 | P |
| | | gi = 2644867/ug = Mm.25263/len = 408/STRA = for | | | | | | | |
| 10 | 94362_at | Cluster Incl AI843682 UI-M-AK1-aer-c-04-0-UI.s1 Mus musculus cDNA, 3 end/clone = UI-M-AK1-aer-c-04-0-UI/ | P | 1428 | P | 1086.3 | P | 1382 | P |
| | | clone_end = 3/gb = AI843682/gi = 5477895/ug = Mm 25265/len = 160/STRA = for | | | | | | | |
| 10 | 94925_at | Cluster Incl AA754865: vu57f07 r1 Mus musculus cDNA, 5 end/clone = IMAGE-1194949/clone_end = 5/gb = AA754865/ | P | 257.2 | P | 213.3 | P | 225.9 | P |
| | | gi = 2802063/ug = Mm.26644/len = 324/STRA = for | | | | | | | |
| 10 | 95525_at | Cluster Incl AI850793 UI-M-BG1-all-e-06-0-UI s1 Mus musculus cDNA, 3 end/clone = UI-M-BG1-all-e-06-0-UI/ | P | 313.4 | P | 184.2 | P | 263.2 | P |
| | | clone_end = 3/gb = AI850793/gi = 5494699/ug = Mm.27592/len = 517/STRA = for | | | | | | | |
| 10 | 95539_at | Cluster Incl AA798971 vv95e03.r1 Mus musculus cDNA, 5 end/clone = IMAGE-1230172/clone_end = 5/gb = AA798971/ | P | 157.4 | P | 190.8 | P | 152.1 | P |
| | | gi = 2861926/ug = Mm.27668/len = 449/STRA = for | | | | | | | |
| 10 | 96146_at | Cluster Incl D83745: Mus musculus mRNA for ANA, complete cds/cds = (173,931)/gb = D83745/gi = 3256228/ug = Mm.2823/ | P | 579.7 | P | 502.3 | P | 457.5 | P |
| | | len = 1380/STRA = for | | | | | | | |
| 10 | 96744_at | Cluster Incl AW061051 UI-M-BH1-akk-f-05-0-UI s2 Mus musculus cDNA, 3 end/clone = UI-M-BH1-akk-f-05-0-UI/ | P | 502.2 | P | 352.9 | P | 336 | P |
| | | clone_end = 3/gb = AW061051/gi = 6008802/ug = Mm 28950/len = 517/STRA = for | | | | | | | |
| 10 | 96754_s_at | Cluster Incl AI844751 UI-M-AL1-ahq-e-01-0-UI.s1 Mus musculus cDNA, 3 end/clone = UI-M-AL1-ahq-e-01-0-UI/ | P | 1108.8 | P | 1012.3 | P | 1064.4 | P |
| | | clone_end = 3/gb = AI844751/gi = 5488657/ug = Mm 28982/len = 437/STRA = for | | | | | | | |
| 10 | 96804_at | Cluster Incl AI854811 UI-M-BH0-aka-g-11-0-UI s1 Mus musculus cDNA, 3 end/clone = UI-M-BH0-aka-g-11-0-UI/ | P | 302.7 | P | 195.8 | P | 354.5 | P |
| | | clone_end = 3/gb = AI854811/gi = 5498717/ug = Mm 29203/len = 366/STRA = rev | | | | | | | |
| 10 | 97380_at | Cluster Incl AI197431 uf13e07.r1 Mus musculus cDNA, 3 end/clone = IMAGE-1851204/clone_end = 5/gb = AI197431/ | P | 378.6 | P | 278.1 | P | 391.1 | P |
| | | gi = 3750037/ug = Mm 29748/len = 331/STRA = for | | | | | | | |
| 10 | 97390_at | Cluster Incl AW125294 UI-M-BH2.1-apy-b-10-0-UI s1 Mus musculus cDNA, 3 end/clone = UI-M-BH2.1-apy-b-10-0-UI/ | P | 270.2 | P | 298 | P | 289.5 | P |
| | | clone_end = 3/gb = AW125294/gi = 6100824/ug = Mm 29800/len = 345/STRA = for | | | | | | | |
| 10 | 97979_at | Cluster Incl AI849126: UI-M-AJ1-agy-a-10-0-UI s1 Mus musculus cDNA, 3 end/clone = UI-M-AJ1-agy-a-10-0-UI/ | P | 224.1 | P | 249.1 | P | 218.1 | P |
| | | clone_end = 3/gb = AI849126/gi = 5493032/ug = Mm 3121/len = 489/STRA = for | | | | | | | |
| 10 | 99499_at | Cluster Incl AB023619 Mus musculus mRNA, for methyltransferase related protein, complete cds/cds = (47,853)/ | P | 284 | P | 280.3 | P | 296.4 | P |
| | | gb = AB023619/gi = 4512324/ug = Mm 41667/len = 887/STRA = for | | | | | | | |
| 10 | 99529_f_at | Cluster Incl AB025011: Mus musculus mRNA for Trif-d, complete cds/cds = (303,590)/gb = AB025011/gi = 4586406/ | P | 128.4 | P | 104.8 | P | 129.5 | P |
| | | ug = Mm.42154/len = 1078/STRA = for | | | | | | | |
| 10 | 100425_at | Cluster Incl U25685: Mus musculus protein tyrosine kinase (syk) mRNA complete cds/cds = (0,1889)/gb = U25685/ | P | 499.3 | P | 492.1 | P | 530.5 | P |
| | | gi = 818860/ug = Mm 4708/len = 1890/STRA = for | | | | | | | |
| 10 | 101944_at | Cluster Incl U89352: Mus musculus lysophospholipase I mRNA, complete cds/cds = (0,692)/gb = U89352/gi = 1864158/ | P | 567.1 | P | 713.9 | P | 780.7 | P |
| | | ug = Mm 95696/len = 693/STRA = for | | | | | | | |
| 10 | 101946_at | Cluster Incl AA840463 vx93d09.r1 Mus musculus cDNA, 5 end/clone = IMAGE-1282769/clone_end = 5/gb = AA840463/ | P | 270.8 | P | 235 | P | 326.8 | P |
| | | gi = 2916122/ug = Mm 95696/len = 430/STRA = for | | | | | | | |
| 10 | 102271_at | Cluster Incl AW125462 UI-M-BH2 2-aqm-a-11-0-UI s1 Mus musculus cDNA, 3 end/close = UI-M-BH2 2-aqm-a-11-0-UI/ | P | 500.4 | P | 559.3 | P | 637.7 | P |
| | | clone_end = 3/gb = AW125462/gi = 6100992/ug = Mm.1144/len = 439/STRA = for | | | | | | | |
| 10 | 103242_at | Cluster Incl AW123834 UI-M-BH2.1-apm-h-06-0-UI s1 Mus musculus cDNA, 3 end/clone = UI-M-BH2 1-apm-h-06-0-UI/ | P | 161.3 | P | 171.9 | P | 82.8 | P |
| | | clone_end = 3/gb = AW123834/gi = 6099364/ug = Mm.20417/len = 403/STRA = for | | | | | | | |
| 10 | 103754_at | Cluster Incl AI847826 UI-M-AM1-afu-a-09-0-UI s1 Mus musculus cDNA, 3 end/clone = UI-M-AM1-afu-a-09-0-UI/ | P | 421.1 | P | 388.8 | P | 403 | P |
| | | clone_end = 3/gb = AI847826/gi = 5491732/ug = Mm 24529/len = 301/STRA = for | | | | | | | |
| 10 | 104447_r_at | Cluster Incl AI595812: uj69f12.x1 Mus musculus cDNA, 3 end/clone = IMAGE-1925231/clone_end = 3/gb = AI595812/ | P | 69.4 | P | 37.3 | P | 50.3 | P |
| | | gi = 4604860/ug = Mm 29409/len = 596/STRA = for | | | | | | | |
| 10 | 92389_at | Cluster Incl AB025411: Mus musculus mRNA for Ten-m2, complete cds/cds = (112,8406)/gb = AB025411/gi = 4760777/ | P | 628.9 | P | 736.2 | P | 711.9 | P |
| | | ug = Mm 40599/len = 8797/STRA = rev | | | | | | | |
| 10 | 93215_at | Cluster Incl AF061346 Tumor necrosis factor induced protein 1/cds = (0,950)/gb = AF061346/gi = 3114712/ug = Mm.57088/ | P | 165.5 | P | 218.2 | P | 276 | P |
| | | len = 951/STRA = for | | | | | | | |
| 10 | 93430_at | Cluster Incl AF000236 Chemokine orphan receptor 1/cds = (88,1176)/gb = AF000236/gi = 2439995/ug = Mm 6522/len = 1861/ | P | 97 | P | 86.1 | P | 86.8 | P |
| | | STRA = for | | | | | | | |
| 10 | 93918_at | Cluster Incl AA673500 vp49g08 r1 Mus musculus cDNA, 3 end/clone = IMAGE-1080062/clone_end = 3/gb = AA673500/ | P | 218.6 | P | 194.8 | P | 202.3 | P |
| | | gi = 2646782/ug = Mm 89836/len = 357/STRA = for | | | | | | | |
| 10 | 94189_at | Cluster Incl AB011665 Mus musculus mRNA for BAZF, complete cds/cds = (24,1448)/gb = AB01165/gi = 3287500/ | P | 440.8 | P | 272.3 | P | 832.4 | P |
| | | ug = Mm 12930/len = 3345/STRA = for | | | | | | | |
| 10 | 95885_at | Cluster Incl AA177621 mt32e09 r1 Mus musculus cDNA, 5 end/clone = IMAGE-622792/clone_end = 5/gb = AA177621/ | P | 129 | P | 131.1 | P | 145.9 | P |
| | | gi = 1758855/ug = Mm 23986/len = 517/STRA = for | | | | | | | |
| 10 | 97104_g_at | Cluster Incl AF031380 Mus musculus KOI-4 gene, partial cds/cds = (0,102)/gb = AF031380/gi = 2623675/ug = Mm 32715/ | P | 264.1 | P | 262.1 | P | 300 | P |
| | | len = 103/STRA = rev | | | | | | | |
| 10 | 97770_s_at | Cluster Incl AA733372 vt74c06 r1 Mus musculus cDNA, 5 end/clone = IMAGE-1176874/clone_end = 5/gb = AA733372/ | P | 148.2 | P | 146.4 | P | 133.5 | P |
| | | gi = 2755039/ug = Mm 4176/len = 316/STRA = for | | | | | | | |
| 10 | 98356_at | Cluster Incl AW125421 UI-M-BH2 3-aqh-f-02-0-UI s1 Mus musculus cDNA, 3 end/clone = UI-M-BH2 3-aqh-f-02-0-UI/ | P | 164.8 | P | 160.3 | P | 174.2 | P |
| | | clone_end = 3/gb = AW125421/gi = 6100951/ug = Mm.43948/len = 570/STRA = rev | | | | | | | |
| 10 | 93567_at | Cluster Incl AW122536 UI-M-BH2 2-aox-c-06-0-UI s1 Mus musculus cDNA, 3 end/clone = UI-M-BH2 2-aox-c-06-0-UI/ | P | 185.3 | P | 188.5 | P | 151.3 | P |
| | | clone_end = 3/gb = AW122536/gi = 6098060/ug = Mm.20399/len = 523/STRA = for | | | | | | | |
| 10 | 97847_at | Cluster Incl AJ237847: Mus musculus mRNA for heterogeneous nuclear ribonucleoprotein G, splice variant 1/ | P | 251.1 | P | 283.7 | P | 257 | A |
| | | cds = (153,1328)/gb = AJ237847/gi = 5579010/ug = Mm 28275/len = 2074/STRA = for | | | | | | | |
| 10 | 102319_at | Cluster Incl AF062484 Mus musculus SDP8 mRNA, complete cds/cds = (64,561)/gb = AF062484/gi = 3126980/ | P | 42.6 | P | 26.6 | P | 10 | P |
| | | ug = Mm.10704/len = 617/STRA = for | | | | | | | |
| 10 | 95573_at | Cluster Incl AW122821: UI-M-BH2 1-aoz-c-02-0-UI s1 Mus musculus cDNA, 3 end/clone = UI-M-BH2 1-aoz-c-02-0-UI/ | A | 383.2 | P | 304.2 | P | 345.4 | P |
| | | clone_end = 3/gb = AW122821/gi = 6098351/ug = Mm 27809/len = 329/STRA = rev | | | | | | | |
| 10 | 98006_at | Cluster Incl D13546 Mus musculus mRNA for DNA polymerase alpha associated subunit, complete cds/cds = (235,2037)/ | P | 181.8 | P | 225.3 | P | 163.7 | A |
| | | gb = D13546/gi = 303658/ug = Mm 320/len = 2352/STRA = for | | | | | | | |
| 10 | 99032_at | Cluster Incl AF009246 Mus musculus ras-related protein (DEXRAS1) mRNA, complete cds/cds = (141,983)/gb = AF009246/ | P | 158.2 | P | 137.1 | P | 154.2 | M |
| | | gi = 2253712/ug = Mm 3903/len = 1623/STRA = for | | | | | | | |
| 10 | 99078_at | Cluster Incl AI839522: UI-M-ANO-acn-d-09-UI s1 Mus musculus cDNA, 3 end/clone = UI-M-ANO-acn-d-09-0-UI/ | P | 394.2 | P | 362.5 | P | 407.2 | A |
| | | clone_end = 3/gb = AI839522/gi = 5473735/ug = Mm 4022/len = 481/STRA = for | | | | | | | |
| 10 | 103681_at | Cluster Incl AW049274: UI-M-BH1-anb-d-07-0-UI s1 Mus musculus cDNA, 3 end/clone = UI-M-BH1-anb-d-07-0-UI/ | P | 356.1 | P | 266.7 | P | 389.2 | A |
| | | clone_end = 3/gb = AW049274/gi = 5909768/ug = Mm 23885/len = 471/STRA = for | | | | | | | |
| 10 | 104396_at | Cluster Incl AI019327 ub 19g08 r1 Mus musculus cDNA, 5 end/clone = IMAGE-1378238/clone_end = 5/gb = AI019327/ | P | 192.8 | P | 151.5 | P | 222.8 | P |
| | | gi = 3233683/ug = Mm 28617/len = 265/STRA = for | | | | | | | |
| 10 | 93680_at | Cluster Incl D89728 Mus musculus mRNA for LOK, complete cds/cds = (99,2999)/gb = D89728/gi = 2723474/ug = Mm 8235/ | P | 229.1 | P | 67.7 | A | 260.4 | P |
| | | len = 5002/STRA = for | | | | | | | |
| 10 | 94709_at | Cluster Incl D88611: Glial cells missing homolog (Drosophila), related sequence 2/cds = (241,1755)/gb = D88611/ | P | 210.4 | P | 110.7 | P | 266.6 | P |
| | | gi = 1769815/ug = Mm 1399/len = 3118/STRA = for | | | | | | | |
| 10 | 99338_at | Cluster Incl AA674798: vm74h03 s1 Mus musculus cDNA, 5 end/clone = IMAGE-1004021/clone_end = 5/gb = AA674798/ | P | 210.5 | P | 145.9 | P | 105.8 | M |
| | | gi = 2652035/ug = Mm.5300/len = 466/STRA = rev | | | | | | | |
| 10 | 93248_at | Cluster Incl AI050598 ub33h10 r1 Mus musculus cDNA, 5 end/clone = IMAGE-1379587/clone_end = 5/gb = AI050598/ | P | 168.2 | P | 177.3 | A | 184.7 | P |
| | | gi = 3299715/ug = Mm.16925/len = 487/STRA = for | | | | | | | |
| 10 | 96678_at | Cluster Incl AW260761 um87b01 y1 Mus musculus cDNA, 5 end/clone = IMAGE-2331721/clone_end = 5/gb = AW260761/ | P | 81.9 | P | −93.5 | P | 107.7 | A |
| | | gi = 6633742/ug = Mm.27427/len = 336/STRA = for | | | | | | | |
| 10 | 96148_at | Cluster Incl AA028728 mi16d09.r1 Mus musculus cDNA, 5 end/clone = IMAGE-463697/clone_end = 5/gb = AA028728/ | P | 137.1 | P | 88.4 | M | 145.1 | P |
| | | gi = 1494856/ug = Mm.3572/len = 1061/STRA = for | | | | | | | |
| 10 | 100570_at | Cluster Incl U27462 Mus musculus BS4 peptide mRNA, complete cds/cds = (247,2280)/gb = U27462/gi = 863013/ | P | 333.6 | P | 363.5 | A | 407 | P |
| | | ug = Mm 5856/len = 3312/STRA = for | | | | | | | |
| 10 | 103863_at | Cluster Incl AW049769 UI-M-BH1-anm-c-05-0-UI.s1 Mus musculus cDNA, 3 end/clone = UI-M-BH1-anm-c-05-0-UI/ | P | 666.2 | P | 644.5 | P | 712.4 | A |
| | | clone_end = 3/gb = AW049769/gi = 5910298/ug = Mm 21104/len = 543/STRA = rev | | | | | | | |
| 10 | 98992_at | Cluster Incl AB030483: Mus musculus mRNA for B9, complete cds/cds = (37,651)/gb = AB030483/gi = 5668700/ | P | 71 | P | 157.9 | P | 124.5 | M |
| | | ug = Mm 37756/len = 817/STRA = for | | | | | | | |
| 10 | 99028_at | Cluster Incl AI788534 uk46b08.x1 Mus musculus cDNA, 3 end/clone = IMAGE-1972023/clone_end = 3/gb = AI788534/ | P | 133.6 | P | 124 | A | 117.8 | A |
| | | gi = 5336250/ug = Mm.38831/len = 682/STRA = for | | | | | | | |
| 10 | 99041_at | Cluster Incl Y09973: M. musculus mRNA for TAF168/cds = (0,1760)/gb = Y09973/gi = 1842205/ug = Mm.39082/len = 1761/ | P | 344.7 | P | 239.4 | P | 276.3 | A |
| | | STRA = for | | | | | | | |
| 10 | 100929_at | Cluster Incl AW046351: UI-M-BH1-ali-a-02-0-UI.s1 Mus musculus cDNA, 3 end/clone = UI-M-BH1-ali-a-02-0-UI/ | A | 127 | P | 61.1 | A | 83.2 | P |
| | | clone_end = 3/gb AW046351/gi = 5906880/ug = Mm 6128/len = 503/STRA = for | | | | | | | |
| 10 | 102724_at | Cluster Incl AI608324: vf77b06 y1 Mus musculus cDNA, 5 end/clone = IMAGE-849779/clone_end = 5/gb = AI608324/ | P | 140.7 | P | 132.6 | A | 131.7 | P |
| | | gi = 4617491/ug = Mm 1436/len = 474/STRA = for | | | | | | | |
| 10 | 103476_at | Cluster Incl AA674794: vm74g09.s1 Mus musculus cDNA, 5 end/clone = IMAGE-1004032/clone_end = 5/gb = AA674794/ | P | 190.2 | P | 235.6 | M | 181.5 | P |
| | | gi = 2652031/ug = Mm 21926/len = 175/STRA = for | | | | | | | |
| 10 | 98306_g_at | Cluster Incl Y11245 M musculus mRNA for winged-helix transcription factor/cds = (79,2352)/gb = Y11245/gi = 2065248/ | P | 443.8 | P | 238.2 | A | 313.4 | M |
| | | ug = Mm 42148/len = 2673/STRA = for | | | | | | | |
| 10 | 100395_at | Cluster Incl X99104 M musculus mRNA for gli2 gene/cds = UNKNOWN/gb = X99104/gi = 1707589/ug = Mm.89929/len = 5849/ | P | 163.6 | P | 156.1 | A | 165 | A |
| | | STRA = for | | | | | | | |
| 10 | 92827_at | Cluster Incl AF064553 Mus musculus NSD1 protein mRNA, complete cds/cds = (1099,8865)/gb = AF064553/gi = 3329464/ | P | 61.5 | P | 8.1 | A | 91.2 | P |
| | | ug = Mm 12964/len = 10019/STRA = for | | | | | | | |
| 10 | 97539_at | Cluster Incl AI854416 UI-M-BH0-alt-b-02-0-UI s1 Mus musculus cDNA, 3 end/clone = UI-M-BH0-alt-b-02-0-UI/clone_end = 3/ | P | 20.9 | P | −72.4 | P | 14.9 | A |
| | | gb = AI854416/gi = 5498322/ug = Mm 33202/len = 504/STRA = for | | | | | | | |
| 10 | 100122_at | Cluster Incl L34290 Mus musculus guanine nucleotide regulatory protein mRNA, complete cds/cds = (30,1091)/gb = L34290/ | P | 212.4 | A | 172.8 | P | 161.3 | P |
| | | gi = 1237255/ug = Mm 4702/len = 1803/STRA = for | | | | | | | |
| 10 | 101003_at | Cluster Incl X53824 Splicing factor, arginine/serine-rich 3 (SRp20)/cds = (62,556)/gb = X53824/gi = 55439/ug = Mm 6787/ | A | 159.2 | P | 99.9 | A | 88.8 | A |
| | | len = 1239/STRA = for | | | | | | | |
| 10 | 102809_s_at | Cluster Incl M12056 Mouse rearranged Ick gene encoding lymphocte-specific protein tyrosine kinase/cds = (163,1692)/ | P | 109.8 | P | 68.5 | A | 99.1 | A |
| | | gb = M12056/gi = 198763/ug = Mm 142/len = 2100/STRA = for | | | | | | | |
| 10 | 103872_r_at | Cluster Incl AA667323: vv29b11.r1 Mus musculus cDNA, 5 end/clone = IMAGE-1223805/clone_end = 5/gb = AA667323/ | P | −41.3 | P | −32.1 | A | 99.1 | A |
| | | gi = 2626024/ug = Mm 21167/len = 210/STRA = for | | | | | | | |
| 10 | 104153_at | Cluster Incl AW047743 UI-M-BH1-alk-b-07-0-UI s1 Mus musculus cDNA, 3 end/clone = UI-M-BH1-alk-b-07-0-UI/ | P | 237.3 | P | 237.9 | A | 280.1 | P |
| | | clone_end = 3/gb = AW047743/gi = 5908272/ug = Mm 22199/len = 339/STRA = for | | | | | | | |
| 10 | 103516_at | Cluster Incl AF031572 Mus musculus seven-pass transmembrane receptor precursor (Celsr1) mRNA, complete cds/ | P | 237 | P | 229.8 | P | 312.1 | A |
| | | cds = (7,9111)/gb = AF031572/gi = 3800735/ug = Mm 22680/len = 10919/STRA = for | | | | | | | |
| 10 | 104176_at | Cluster Incl AI850941 UI-M-BH0-akd-d-10-0-UI s1 Mus musculus cDNA, 3 end/clone = UI-M-BH0-akd-d10-0-UI/ | P | 144.1 | P | 122.3 | A | 152.4 | P |
| | | clone_end = 3/gb = AI850941/gi = 5494847/ug = Mm 27261/len = 377/STRA = for | | | | | | | |
| 10 | 92914_at | Cluster Incl M18400: Mouse homeo box (Hox2 3) region/cds = (0,253)/gb = M18400/gi = 193939/ug = Mm 4822/len = 254/ | P | 309.1 | P | 192.6 | P | 163 | A |
| | | STRA = for | | | | | | | |
| 10 | 93212_at | Cluster Incl Z97207 Mus musculus partial mRNA, for B-IND1 protein (B-ind1 gene)/cds = (0,570)/gb = Z97207/gi = 5777593/ | P | 75.5 | P | 27.2 | A | 80.7 | A |
| | | ug = Mm.57023/len = 571/STRA = for | | | | | | | |
| 10 | 93481_at | Cluster Incl AI846720 UI-M-AN1-afi-h-09-0-UI s1 Mus musculus cDNA, 3 end/clone = UI-M-AN1-afi-b-09-0-UI/ | P | 248.9 | M | 417.8 | P | 196.4 | P |
| | | clone_end = 3/gb = AI846720/gi = 5490626/ug Mm 7124/len = 161/STRA = for | | | | | | | |
| 10 | 98818_at | Cluster Incl X04435: Glucocorticold receptor 1/cds = (18,2369)/gb = X04435/gi = 51057/ug = Mm 4918/len = 2575/STRA = for | A | 48.8 | P | 41.8 | A | 72.7 | P |
| 10 | 93056_g_at | Cluster Incl AW049376 UI-M-BH1-ane-c-05-0-UI s1 Mus musculus cDNA, 3 end/clone = UI-M-BH1-ane-c-05-0-UI/ | P | 78.6 | P | −0.1 | A | 13.8 | M |
| | | clone_end = 3/gb = AW049376/gi = 5909905/ug = Mm 15351/len = 492/STRA = for | | | | | | | |
| 10 | 97822_at | Cluster Incl AW122689 UI-M-BH2 2-aot-a-10-0-UI s1 Mus musculus cDNA, 3 end/clone = UI-M-BH2 2-aot-a-10-0-UI/ | P | 529.7 | P | 460.8 | A | 505.4 | A |
| | | clone_end = 3/gb = AW122689/gi = 6098319/ug = Mm 28202/len = 467/STRA = rev | | | | | | | |
| 10 | 98886_at | Cluster Incl AI835412 UI-M-AQ0-aaj-d-12-0-UI s1 Mus musculus cDNA, 3 end/clone = UI-M-AQ0-aaj-d-12-0-UI/ | A | 361.9 | P | 377.8 | P | 375.5 | P |
| | | clone_end = 3/gb = AI835412/gi = 5469625/ug = Mm 28536/len = 355/STRA = for | | | | | | | |
| 10 | 95122_g_at | Cluster Incl AW060791 UI-M-BH1-anu-f-12-0-UI s1 Mus musculus cDNA, 3 end/clone = UI-M-BH1-anu-f-12-0-UI/ | P | 89.9 | P | 94.3 | A | 119.9 | A |
| | | clone_end = 3/gb = AW060791/gi = 6008542/ug = Mm 29390/len = 519/STRA = for | | | | | | | |
| 10 | 99163_at | Cluster Incl AI844232 UI-M-AL1-ahk-f-04-0-UI.s1 Mus musculus cDNA, 3 end/clone = UI-M-AL1-ahk-f-04-0-UI/ | P | 202.9 | P | 101.5 | A | 147.1 | A |
| | | clone_end = 3/gb = AI844232/gi = 5488138/ug = Mm 41671/len = 242/STRA = for | | | | | | | |
| 10 | 94401_s_at | Cluster Incl AA986548 ue14g03 x1 Mus musculus cDNA, 3 end/clone = IMAGE-1480372/clone_end = 3/gb AA986548/ | P | 27.9 | P | 29.4 | M | 36.1 | A |
| | | gi = 3168202/ug = Mm 25793/len = 543/STRA = for | | | | | | | |
| 10 | 98003_at | Cluster Incl AF038149 Mus musculus paired immonoglobutin-like receptor B (Pirb) gene, complete cds/cds = (0,2525)/ | P | 391.8 | P | 282.1 | M | 387.3 | M |
| | | gb = AF038149/gi = 3135465/ug = Mm.3189/len = 2526/STRA = for | | | | | | | |
| 10 | 101945_g_at | Cluster Incl U89352: Mus musculus lysophospholipase I mRNA, complete cds/cds = (0,692)/gb = U89352/gi = 1864158/ | P | 60.9 | P | 40.3 | A | 43.4 | A |
| | | ug = Mm.95696/len = 693/STRA = for | | | | | | | |
| 10 | 102277_at | Cluster Incl M36514.Mouse zinc finger protein (mkr3) mRNA, end/cds = (0,1286)/gb = M36514/gi = 191507/ug = Mm 1176/ | P | 94.3 | P | 80.3 | P | 87.4 | M |
| | | len = 2470/STRA = for | | | | | | | |
| 10 | 103272_at | Cluster Incl AF091998: Mus musculus calpain Lp82 mRNA, complete cds/cds = (37,2166)/gb = AF091998/gi = 3661584/ | P | 357.8 | A | 332.3 | M | 266.1 | A |
| | | ug = Mm 20863/len = 2334/STRA = for | | | | | | | |
| 10 | 93410_at | Cluster Incl AW120840 UI-M-BH2 3-aoa-e-01-0-UI s1 Mus musculus cDNA, 3 end/clone = UI-M-BH2 3-aoa-e-01-0-UI/ | P | 233.7 | A | 221.7 | A | 202.2 | P |
| | | clone_end = 3/gb = AW120840/gi = 6096173/ug = Mm.6378/len = 371/STRA = for | | | | | | | |
| 10 | 100371_at | Cluster Incl U65316: Mus musculus hnRNP A1 (Hnrpa1) gene, alternative splicing region, exon 6 to partial exon 8, and | P | 86.5 | P | 138 | P | 118.2 | M |
| | | partial cds/cds = (0,381)/gb = U65316/gi = 1507693/ug Mm.88834/len = 382/STRA = for | | | | | | | |
| 10 | 92559_at | Cluster Incl U12884: Mus musculus C57BL/6 vascuar cell adhesion molecule-1 truncated form T-VCAM-1 (VCAM-1) | P | 31.1 | P | 6.3 | A | 2.6 | A |
| | | mRNA, complete cds/cds = (43,1080)/gb = U12884/gi = 531884/ug Mm.1021/len = 1332/STRA = for | | | | | | | |
| 10 | 92857_at | Cluster Incl AI853960 UI-M-BH0-aiv-d-02-0-UI s1 Mus musculus cDNA, 3 end/clone = UI-M-BH0-aiv-d-02-0-UI/ | A | 64.1 | P | 0.2 | A | 311.1 | P |
| | | clone_end = 3/gb = AI853960/gi = 5497866/ug = Mm.13917/len = 388/STRA = for | | | | | | | |
| 10 | 93091_s_at | Cluster Incl M63503: Mouse keratinocyte growth factor receptor mRNA, complete cds/cds = (675,2798)/gb = M63503/ | P | 167.8 | A | −125.5 | A | −94 | P |
| | | gi = 198593/ug Mm 16340/len 3037/STRA = for | | | | | | | |
| 10 | 97860_at | Cluster Incl AJ002198.Nucleosome assembly protein 1 like 4/cds = (66,1193)/gb = AJ002198/gi = 3319976/ug Mm 2831/ | M | 488.8 | P | 443.5 | P | 551.1 | A |
| | | len = 2301/STRA = for | | | | | | | |
| 10 | 98016_at | Cluster Incl AA981266 vx80f03 r1 Mus musculus cDNA, 5 end/clone = IMAGE-1279613/clone_end = 5/gb = AA981268/ | A | 161.2 | P | 131.4 | P | 120.3 | A |
| | | gi = 3159804/ug = Mm 3235/len = 424/STRA = for | | | | | | | |
| 10 | 102696_s_at | Cluster Incl AI747899: ul03g02.y1 Mus musculus cDNA, 5 end/clone = IMAGE-2065010/clone_end = 5/gb = AI747899/ | P | 71.1 | A | 93.9 | P | 43.4 | A |
| | | gi = 5126163/ug = Mm.14147/len = 564/STRA = rev | | | | | | | |
| 10 | 102699_at | Cluster Incl J03368: Myxovirus (influenza virus) resistance 2/cds = UNKNOWN/gb = J03368/gi = 199921/ug = Mm.14157/ | P | 289 | P | 240.6 | A | 184 | M |
| | | len = 2426/STRA = for | | | | | | | |
| 10 | 103928_at | Cluster Incl AI788018 uf16c04.y1 Mus musculus cDNA, 5 end/clone = IMAGE-2087718/clone_end = 5/gb = AI788018/ | A | 132.6 | A | 134.6 | P | 122.7 | M |
| | | gi = 5335734/ug = Mm 25096/len = 488/STRA = for | | | | | | | |
| 10 | 104170_at | Cluster Incl AF003115 Mus musculus JIP-1 (JIP-1) mRNA, complete cds/cds = (137,2119)/gb = AF03115/gi = 2316073/ | A | 534.1 | A | 630.5 | P | 219.8 | A |
| | | ug = Mm.2720/len = 2790/STRA = for | | | | | | | |
| 10 | 93400_at | Cluster Incl U88990 Apoptosis Inhibitor 3/cds = (671,2161)/gb = U88990/gi = 2138318/ug = Mm 6299/len = 2691/STRA = for | A | 72.5 | A | 94.7 | A | 98.8 | A |
| 10 | 93911_at | Cluster Incl AI315090: uj23g10 x1 Mus musculus cDNA, 3 end/clone = IMAGE-1920834/clone_end = 3/gb = AI315090/ | A | 238 | P | 106.7 | P | 155.8 | P |
| | | gi = 4030357/ug = Mm.89567/len = 467/STRA = for | | | | | | | |
| 10 | 101231_at | Cluster Incl C78334 C78334 Mus musculus cDNA, 3 end/clone = J0047A07/clone_end = 3/gb = C78334/gi = 2518664/ | A | 141.7 | P | 78.9 | A | 106.7 | P |
| | | ug = Mm.5703/len = 571/STRA = rev | | | | | | | |
| 10 | 102093_f_at | Cluster Incl AI461979: ub69a03 x1 Mus musculus cDNA, 3 end/clone = IMAGE-1382965/clone_end = 3/gb = AI461979/ | P | 81.4 | P | 36.5 | A | 63.7 | P |
| | | gi = 4316009/ug = Mm 42326/len = 272/STRA = rev | | | | | | | |
| 10 | 102163_at | Cluster Incl X60388 M musculus SmN pseudogene/cds = (0,725)/gb = X60388/gi = 54118/ug = Mm 46814/len = 726/ | A | 389.3 | P | 273.6 | M | 377.3 | M |
| | | STRA = for | | | | | | | |
| 10 | 92841_l_at | Cluster Incl X51429 Chromogranin B/cds = (52,2085)/gb = X51429/gi = 50408/ug = Mm 1339/len = 2340/STRA = for | A | 90.7 | A | 100.9 | A | 80.9 | A |
| 10 | 96046_at | Cluster Incl X96207 M musculus mRNA for histone deacetylase/cds = (17,1465) gb = X98207/gi = 1771285/ug = Mm 2602/ | M | 179.6 | P | 184.2 | M | 301.5 | A |
| | | len = 1977/STRA = for | | | | | | | |
| 10 | 102758_at | Cluster Incl AJ006486 Mus musculus mRNA for RNA binding protein/cds = (117,3470)/gb = AJ006486/gi = 4883436/ | A | 77.5 | P | 26.9 | P | −2.9 | A |
| | | ug = Mm 12931/len = 3593/STRA = for | | | | | | | |
| 10 | 103393_at | Cluster Incl AA738776 W67d07 r1 Mus musculus cDNA, 5 end/clone = IMAGE-1227469/clone_end = 5/gb = AA738776/ | P | 82.3 | P | 27.2 | A | 81.6 | P |
| | | gi = 2776028/ug = Mm 18659/len = 385/STRA = for | | | | | | | |
| 10 | 102631_at | Cluster Incl AB008674 Mus musculus mRNA for mBlm, complete cds/cds = (342,4592)/gb = AB008674/gi = 3370995/ | P | 107.6 | P | 39.5 | A | 54.7 | M |
| | | ug = Mm 12932/len = 4763/STRA = for | | | | | | | |
| 10 | 103200_at | Cluster Incl AA711773 vu58g05 r1 Mus musculus cDNA, 5 end/clone = IMAGE-1195640/clone_end = 5/gb = AA711773/ | M | 82.9 | P | 42.4 | A | 34.3 | A |
| | | gi = 2721691/ug = Mm 1902/len = 473/STRA = for | | | | | | | |
| 10 | 95244_at | Cluster Incl D10632 Zinc finger protein 40/cds = (222,2408)/gb = D10632/gi = 286104/ug = Mm 21025/len = 2754/STRA = for | A | 44.8 | P | 25.7 | A | 33.6 | A |
| 10 | 101677_at | Cluster Incl AI450336 mu27g11 x1 Mus musculus cDNA, 3 end/clone = IMAGE-640676/clone_end = 3/gb = AI450336/ | A | 149.1 | P | 57 | A | 96.7 | P |
| | | gi = 4296290/ug = Mm 32307/len = 451/STRA = rev | | | | | | | |
| 11 | 92553_at | Cluster Incl AB025408 Mus musculus mRNA for sid478p, complete cds/cds = (74,922)/gb = AB025408/gi = 5931564/ | P | 903.3 | P | 1064.9 | P | 1149.7 | P |
| | | ug = Mm.1017/len = 1056/STRA = for | | | | | | | |
| 11 | 92589_at | Cluster Incl AI846545 UI-M-AN1-aff-g-12-0-UI s1 Mus musculus cDNA, 3 end/clone = UI-M-AN1-aff-g-12-0-UI/ | P | 740.6 | P | 927.7 | P | 895.8 | P |
| | | clone_end = 3/gb = AI846545/gi = 5490451/ug = Mm 10623/len = 354/STRA = for | | | | | | | |
| 11 | 92595_r_at | Cluster Incl M61215 Mus musculus ferrochelatase mRNA, complete cds/cds = (92,1354)/gb = M61215/gi = 19362/ | P | 1251 | P | 136 | P | 135.7 | P |
| | | ug = Mm.1070/len = 2897/STRA = for | | | | | | | |
| 11 | 92608_at | Cluster Incl D88793 Cysteine rich protein/cds = (130,711)/gb = D88793/gi = 1695703/ug = Mm 10919/len = 938/STRA = for | P | 789.3 | P | 1061.8 | P | 961.1 | P |
| 11 | 92632_at | Cluster Incl AI842328: UI-M-AM1-afz-b-11-0-UI s1 Mus musculus cDNA, 3 end/clone = UI-M-AM1-afz-b-11-0-UI/ | P | 892.1 | P | 1134.6 | P | 988.3 | P |
| | | clone_end = 3/gb = AI842328/gi = 5476506/ug = Mm 1147/len = 389/STRA = for | | | | | | | |
| 11 | 92636_f_at | Cluster Incl U11027 Mus musculus C57BL/6J sec61 protein complex gamma subunit mRNA, complete cds/cds = (3,209)/ | P | 1857.3 | P | 1998.2 | P | 2004.8 | P |
| | | gb = U11027/gi = 506791/ug = Mm.1164/len = 367/STRA = for | | | | | | | |
| 11 | 92646_at | Cluster Incl U84903 Ribosomal protein L23/cds = (61,501)/gb = U84903/gi = 2565133/ug = Mm 12144/len = 582/STRA = for | P | 848 | P | 1148.2 | P | 1288.5 | P |
| 11 | 92798_at | Cluster Incl AA870675 vq24f11.r1 Mus musculus cDNA, 5 end/clone = IMAGE-1095213/clone_end = 5/gb = AA870675/ | P | 2424.2 | P | 2402.4 | P | 2588.1 | P |
| | | gi = 2966120/ug = Mm.12677/len = 405/STRA = for | | | | | | | |
| 11 | 92799_g_at | Cluster Incl AA870675 vq24f11.r1 Mus musculus cDNA, 5 end/clone = IMAGE-1095213/clone_end = 5/gb = AA870675/ | P | 2542.8 | P | 2951.5 | P | 2914.8 | P |
| | | gi = 2966120/ug = Mm 12677/len = 405/STRA = for | | | | | | | |
| 11 | 92810_at | Cluster Incl AI842259 UI-M-AI1-afp-f-09-0-UI s1 Mus musculus cDNA, 3 end/clone = UI-M-AI1 -afp-f-09-0-UI/clone_end = 3/ | P | 446.9 | P | 449.1 | P | 385 | P |
| | | gb = AI842259/gi = 5476472/ug = Mm.12775/len = 389/STRA = for | | | | | | | |
| 11 | 92845_at | Cluster Incl AI843232 UI-M-AK1-aet-h-03-0-UI s1 Mus musculus cDNA, 3 end/clone = UI-M-AK1-aet-h-03-0-UI/ | P | 405.9 | P | 550.5 | P | 330.8 | P |
| | | clone_end = 3/gb = AI843232/gi 5477445/ug = Mm 13445/len = 396/STRA = for | | | | | | | |
| 11 | 93057_at | Cluster Incl AW122989 UI-M-BH2.1-apb-b-08-0-UI s1 Mus musculus cDNA, 3 end/clone = UI-M-BH2 1-apb-b-08-0-UI/ | P | 2744.6 | P | 3322 | P | 3386.8 | P |
| | | clone_end = 3/gb = AW122989/gi = 6098519/ug = Mm 1538/len = 479/STRA = for | | | | | | | |
| 11 | 93094_at | Cluster Incl U88588 Cerebellar degeneration-related 2/cds = (153,1520)/gb = U88588/gi = 1857920/ug = Mm 1640/len = 2432/ | P | 176.4 | P | 261.4 | P | 172.1 | P |
| | | STRA = for | | | | | | | |
| 11 | 93102_f_at | Cluster Incl U20365: Actin, gamma 2, smooth muscle, enteric/cds = (36,1217)/gb = U20365/gi = 950001/ug = Mm 16562/ | P | 1283 | P | 1797.1 | P | 1668.8 | P |
| | | len = 1232/STRA = for | | | | | | | |
| 11 | 93119_at | Cluster Incl X53157 Mouse mRNA for mitochondrial cytochrome c oxidase subunit Vb/cds = (12,398)/gb = X53157/gi = 50530/ | P | 2516.4 | P | 2830.3 | P | 2909 | P |
| | | ug = Mm 16769/len = 475/STRA = for | | | | | | | |
| 11 | 93126_at | Cluster Incl X04591 Mouse mRNA 3 region for brain creatine kinase B/cds = (0,86) gb = X04591/gi = 50413/ug = Mm 16831/ | P | 648.9 | P | 948.5 | P | 1102.7 | P |
| | | len = 285/STRA = for | | | | | | | |
| 11 | 93276_at | Cluster Incl U90123.Hematological and neurological and neurological expressed sequence 1/cds = (95,559) | P | 1107.5 | P | 1240 | P | 1222.5 | P |
| | | gb = U90123/gi = 1864164/ | | | | | | | |
| | | ug = Mm 1775/len = 1384/STRA = for | | | | | | | |
| 11 | 93356_at | Cluster Incl D26091 Mus musculus mRNA for mCDC47, complete cds/cds = (106,2265)/gb = D26091/gi = 1136746/ | P | 461.6 | P | 560.9 | P | 428.3 | P |
| | | ug = Mm.18923/len = 2396/STRA = for | | | | | | | |
| 11 | 93495_at | Cluster Incl U96746 Mus musculus antioxidant enzyme AOE372 mRNA, complete cds/cds = (90,914) gb = U96746/ | P | 601.9 | P | 666.3 | P | 403.8 | P |
| | | gi = 2104954/ug = Mm 19127/len = 977/STRA = for | | | | | | | |
| 11 | 93539_at | Cluster Incl AI118854: uc14105.r1 Mus musculus cDNA, 5 end/clone = IMAGE-1397985/clone_end = 5/gb = AI118854/ | P | 486.5 | P | 710.8 | P | 477.4 | P |
| | | gi = 3519178/ug = Mm.2000/len = 281/STRA = for | | | | | | | |
| 11 | 93575_at | Cluster Incl AF051102: Mus musculus gamma glutamyl hydrolase precursor, mRNA, complete cds/cds = (90,1043)/ | P | 504.8 | P | 524.4 | P | 499.9 | P |
| | | gb = AF051102/gi = 3831561/ug = Mm 20461/len = 1295/STRA = for | | | | | | | |
| 11 | 93812_at | Cluster Incl U53455 Mus musculus chloride ion current inducer protein (GLCI) mRNA, partial cds/cds = (0,710)/gb = U53455/ | P | 583.1 | P | 839.2 | P | 619.7 | P |
| | | gi = 1399647/ug = Mm.21482/len = 711/STRA = for | | | | | | | |
| 11 | 93830_at | Cluster Incl AI851199 UI-M-BH0-ajx-e-10-0-UI s1 Mus musculus cDNA, 3 end/clone = UI-M-BH0-ajx-e-10-0-UI/ | P | 2447.2 | P | 2906.9 | P | 2718 | P |
| | | clone_end = 3/gb = AI851199/gi = 5495105/ug = Mm.21559/len = 301/STRA = for | | | | | | | |
| 11 | 93839_at | Cluster Incl AI854888 UI-M-BH0-akb-g-05-0-UI.s1 Mus musculus cDNA, 3 end/clone = UI-M-BH0-akb-g-05-0-UI/ | P | 831.9 | P | 1024.1 | P | 1068.7 | P |
| | | clone_end = 3/gb = AI854888/gi = 5498794/ug = Mm 21606/len = 468/STRA = for | | | | | | | |
| 11 | 93984_at | Cluster Incl AF002718 Mus musculus ATPase inhibitor (IF1) mRNA, complete cds/cds = (30,350)/gb = AF002718/ | P | 2203.5 | P | 2926.4 | P | 2376.6 | P |
| | | gi = 2367440/ug = Mm 2171/len = 509/STRA = for | | | | | | | |
| 11 | 94032_at | Cluster Incl AI845103 UI-M-BG0-ahs-a-01-0-UI s1 Mus musculus cDNA, 3 end/clone = UI-M-BG0-ahs-a-01-0-UI/ | P | 806.8 | P | 1042.6 | P | 1008.3 | P |
| | | clone_end = 3/gb = AI845103/gi = 5489009/ug = Mm 21905/len = 309/STRA = for | | | | | | | |
| 11 | 94038_at | Cluster Incl AA675468: vr72c04 s1 Mus musculus cDNA, 5 end/clone = IMAGE-1134246/clone_end = 5/gb = AA675468/ | P | 1087.1 | P | 1305 | P | 1477.7 | P |
| | | gi = 2652705/ug = Mm.21932/len = 540/STRA = for | | | | | | | |
| 11 | 94047_at | Cluster Incl AW122935 UI-M-BH2.1-apa-h-12-0-UI s1 Mus musculus cDNA, 3 end/clone = UI-M-BH2.1-apa-h-12-0-UI/ | P | 665 | P | 801.1 | P | 662.2 | P |
| | | clone_end = 3/gb = AW122935/gi = 6098465/ug = Mm 21976/len = 365/STRA = for | | | | | | | |
| 11 | 94206_at | Cluster Incl AC002397 Mouse chromosome 6 Bac-284H12 (Research Genetics mouse BAC library) complete sequence/ | P | 856.1 | P | 1349.8 | P | 1052.6 | P |
| | | cds = (108,488)/gb = AC002397/gi = 3287367/ug = Mm 22195/len = 568/STRA = for | | | | | | | |
| 11 | 94210_at | Cluster Incl AW121333 UI-M-BH2.2-aom-b-04-0-UI.s1 Mus musculus cDNA, 3 end/clone = UI-M-BH2.2-aom-b-04-0-UI/ | P | 1005.7 | P | 1387.6 | P | 1286.3 | P |
| | | clone_end = 3/gb = AW121333/gi = 6096666/ug = Mm 22201/len = 494/STRA = for | | | | | | | |
| 11 | 94229_at | Cluster Incl AW124489: UI-M-BH2.1-apo-a-01-0-UI s1 Mus musculus cDNA, 3 end/clone = UI-M-BH2.1-apo-a-01-0-UI/ | P | 450.1 | P | 618.6 | P | 686.4 | P |
| | | clone_end = 3/gb = AW124489/gi = 6099984/ug = Mm 22281/len = 513/STRA = for | | | | | | | |
| 11 | 94252_at | Cluster Incl AJ006587 Mus musculus mRNA for translation initiation factor eIF2 gamma X/cds = (8,1426)/gb = AJ006587/ | P | 2025.5 | P | 2761.5 | P | 2000.7 | P |
| | | gi = 3790183/ug = Mm 22387/len = 1787/STRA = for | | | | | | | |
| 11 | 94266_at | Cluster Incl AW121737: UI-M-BH2-3-aoe-b-10-0-UI s1 Mus musculus cDNA, 3 end/clone = UI-M-BH2-3-aoe-b-10-0-UI/ | P | 1077.3 | P | 1415.5 | P | 1266.3 | P |
| | | clone_end = 3/gb = AW121737/gi = 6097070/ug = Mm 22469/len = 365/STRA = for | | | | | | | |
| 11 | 94811_s_at | Cluster Incl AJ002366 Mus musculus mRNA for transcription factor TFIIH, 62 kD subunit/cds = (44,1687)/gb = AJ002266/ | P | 151.6 | P | 151.9 | P | 191.7 | P |
| | | gi = 2582796/ug = Mm 22700/len = 2471/STRA = for | | | | | | | |
| 11 | 94817_at | Cluster Incl X60676 M musculus HSP47 mRNA/cds = (256,1509)/gb = X60676/gi = 51449/ug = Mm 22708/len = 2273/ | P | 2365.6 | P | 2815.6 | P | 2835.7 | P |
| | | STRA = for | | | | | | | |
| 11 | 94820_r_at | Cluster Incl AF0058886 Mus musculus cyclin I mRNA, complete cds/cds = (286,1419)/gb = AF005886/gi = 4101686/ | P | 823.7 | P | 1133.2 | P | 889 | P |
| | | ug = Mm 22711/len = 1548/STRA = for | | | | | | | |
| 11 | 94915_at | Cluster Incl X58990 Mouse mCyp-S1 mRNA for cyclophilin CyP-S1/cds = (166,792)/gb = X58990/gi = 53034/ug = Mm 2412/ | P | 1659.5 | P | 2001.8 | P | 2230.1 | P |
| | | len = 998/STRA = for | | | | | | | |
| 11 | 95430_f_at | Cluster Incl AI854154 UI-M-BH0-ajw-h-03-0-UI.s1 Mus musculus cDNA, 3 end/clone = UI-M-BH0-ajw-h-03-0-UI/ | P | 581 | P | 833.1 | P | 715.6 | P |
| | | clone_end = 3/gb = AI854154/gi = 5498060/ug = Mm 24460/len = 561/STRA = for | | | | | | | |
| 11 | 95493_at | Cluster Incl X66405 Procollagen, type VI, alpha 1/cds = (131,3208)/gb = X66405/gi = 50478/ug = Mm 2509/len = 3991/ | P | 2702.4 | P | 3146.9 | P | 3672.4 | P |
| | | STRA = for | | | | | | | |
| 11 | 96057_at | Cluster Incl AI647493 uk42h02.x1 Mus musculus cDNA, 3 end/clone = IMAGE-1971699/clone_end = 3/gb = AI647493/ | P | 455.1 | P | 618.6 | P | 678.8 | P |
| | | gi = 4726171/ug = Mm 2621/len = 761/STRA = for | | | | | | | |
| 11 | 96058_s_at | Cluster Incl U07235 Mus musculus aldehyde dehydrogenase (ALDH2) mRNA, nuclear gene encoding mitochondrial | P | 330.2 | P | 444.7 | P | 415.7 | P |
| | | protein, complete cds/cds = (105,1664)/gb = U07235/gi = 466253/ug = Mm 2621/len = 1954/STRA = for | | | | | | | |
| 11 | 96059_at | Cluster Incl AA823336 vp35g10 r1 Mus musculus cDNA, 5 end/clone = IMAGE-1078722/clone_end = 5/gb = AA823336/ | P | 356.3 | P | 482.5 | P | 449.7 | P |
| | | gi = 2893204/ug = Mm 26217/len = 366/STRA = for | | | | | | | |
| 11 | 96086_at | Cluster Incl AW122530 UI-M-BH2.2-aox-b-12-0-UI s1 Mus musculus cDNA, 3 end/clone = UI-M-BH2.2-aox-b-12-0-UI/ | P | 371.9 | P | 472.8 | P | 398.7 | P |
| | | clone_end = 3/gb = AW122530/gi = 6098054/ug = Mm 26680/len = 206/STRA = for | | | | | | | |
| 11 | 96115_at | Cluster Incl U28168: Adenomatosis polyposis coli/cds = (35,592)/gb = U28168/gi = 1142715/ug = Mm 2698/len = 719/ | P | 2246.1 | P | 2880.4 | P | 2708.9 | P |
| | | STRA = for | | | | | | | |
| 11 | 96617_at | Cluster Incl AI844737 UI-M-AL1-ahq-c-11-0-UI s1 Mus musculus cDNA, 3 end/clone = UI-M-AL1-ahq-c-11-0-UI/ | P | 475.3 | P | 556.2 | P | 627.2 | P |
| | | clone_end = 3/gb = AI844737/gi = 5488643/ug = Mm 27128/len = 178/STRA = for | | | | | | | |
| 11 | 96652_at | Cluster Incl AI849911: UI-M-BG0-ahz-d-03-0-UI s1 Mus musculus cDNA, 3 end/clone = UI-M-BG0-ahz-d-03-0-UI/ | P | 476.5 | P | 526.2 | P | 597.5 | P |
| | | clone_end = 3/gb = AI849911/gi = 5493817/ug = Mm 27332/len = 385/STRA = for | | | | | | | |
| 11 | 96661_at | Cluster Incl AA734806 vp38b03 r1 Mus musculus cDNA, 5 end/clone = IMAGE-1078925/clone_end = 5/gb = AA734806/ | P | 1014.8 | P | 1126.9 | P | 1338.7 | P |
| | | gi = 2756473/ug = Mm.27357/len = 446/STRA = for | | | | | | | |
| 11 | 96667_at | Cluster Incl AI851542 UI-M-BH0-aki-d-01-0-UI.s1 Mus musculus cDNA, 3 end/clone = UI-M-BH0-aki-d-01-0-UI/ | P | 424.5 | P | 447.1 | P | 219.1 | P |
| | | clone_end = 3/gb = AI851542/gi = 5495448/ug = Mm.27389/len = 477/STRA = for | | | | | | | |
| 11 | 96668_at | Cluster Incl AI845867 UI-M-AO1-aeg-g-01-0-UI s1 Mus musculus cDNA, 3 end/clone = UI-M-AO1-aeg-g-01-0-UI/ | P | 756.6 | P | 978.8 | P | 1094.3 | P |
| | | clone_end = 3/gb = AI845867/gi = 5489773/ug = Mm 27393/len = 345/STRA = for | | | | | | | |
| 11 | 98710_at | Cluster Incl AI854262 UI-M-BH0-aja-b-11-0-UI s1 Mus musculus cDNA, 3 end/clone = UI-M-BH0-aja-b-11-0-UI/ | P | 1259.5 | P | 1615.3 | P | 1193.1 | P |
| | | clone_end = 3/gb = AI854262/gi = 5498168/ug = Mm 27624/len = 547/STRA = for | | | | | | | |
| 11 | 97268_i_at | Cluster Incl AI848699 UI-M-AH1-agv-g-05-0-UI.s1 Mus musculus cDNA, 3 end/clone = UI-M-AH1-agv-g-05-0-UI/ | P | 931.8 | P | 1264.7 | P | 1058.3 | P |
| | | clone_end = 3/gb = AI848699/gi = 5492600/ug = Mm 27904/len = 531/STRA = for | | | | | | | |
| 11 | 97269_f_at | Cluster Incl AI848699.UI-M-AH1-agv-g-05-0-UI.s1 Mus musculus cDNA, 3 end/clone = UI-M-AH1-agv-g-05-0-UI/ | P | 1511.5 | P | 1988 | P | 1306.2 | P |
| | | clone_end = 3/gb = AI848699/gi = 5492600/ug = Mm.27904/len = 531/STRA = for | | | | | | | |
| 11 | 97797_at | Cluster Incl AW123142.UI-M-BH2.1-apd-b-05-0-UI s1 Mus musculus cDNA, 3 end/clone = UI-M-BH2.1-apd-b-05-0-UI/ | P | 694.7 | P | 809.3 | P | 944.2 | P |
| | | clone_end = 3/gb = AW123142/gi = 6098672/ug = Mm 28111/len = 572/STRA = for | | | | | | | |
| 11 | 97820_at | Cluster Incl AB027012 Mus musculus galK mRNA for galactokinase, complete cds/cds = (0,1175)/gb = AB027012/ | P | 218.8 | P | 298.9 | P | 252.3 | P |
| | | gi = 5931607/ug = Mm.2820/len = 1293/STRA = for | | | | | | | |
| 11 | 97828_at | Cluster Incl AF033115 Mus musculus proapoptotic protein (Siva) gene, complete cds/cds = (0,527)/gb = AF0033115/ | P | 451.4 | P | 589.4 | P | 406.4 | P |
| | | gi = 2921301/ug = Mm 28216/len = 528/STRA = for | | | | | | | |
| 11 | 97863_at | Cluster Incl AW125274: UI-M-BH2.1-aps-h-08-0-UI.s1 Mus musculus cDNA, 3 end/clone = UI-M-BH2.1-aps-h-08-0-UI/ | P | 379.9 | P | 469.4 | P | 467.8 | P |
| | | clone_end = 3/gb = AW125274/gi = 6100804/ug = Mm.28326/len = 148/STRA = for | | | | | | | |
| 11 | 97866_at | Cluster Incl AI842858: UI-M-AN1-1-afb-c-09-0-UI.s1 Mus musculus cDNA, 3 end/clone = UI-M-AN1-afb-c-09-0-UI/ | P | 425.8 | P | 420.7 | P | 376.4 | P |
| | | clone_end = 3/gb = AI842858/gi = 5477071/ug = Mm 28327/len = 254/STRA = for | | | | | | | |
| 11 | 97909_at | Cluster Incl AI838080 UI-M-AL0-abv-e-12-0-UI.s1 Mus musculus cDNA, 3 end/clone = UI-M-AL0-abv-e-12-0-UI/ | P | 2525.8 | P | 3719.1 | P | 1512 | P |
| | | clone_end = 3/gb = AI838080/gi = 5472293/ug = Mm.28479/len = 226/STRA = for | | | | | | | |
| 11 | 98953_at | Cluster Incl AW048032 UI-M-BH1-alv-b-08-0-UI s1 Mus musculus cDNA, 3 end/clone = UI-M-BH1-alv-b-08-0-UI/ | P | 573.9 | P | 514.1 | P | 548.3 | P |
| | | clone_end = 3/gb = AW048032/gi = 5908526/ug = Mm.28753/len = 587/STRA = for | | | | | | | |
| 11 | 94499_at | Cluster Incl AI835427 UI-M-AQ0-aaj-f-10-0-UI s1 Mus musculus cDNA, 3 end/clone = UI-M-AQ0-aaj-f-10-0-UI/ | P | 584.7 | P | 516.6 | P | 475.2 | P |
| | | clone_end = 3/gb = AI835427/gi = 5469640/ug = Mm 28938/len = 236/STRA = for | | | | | | | |
| 11 | 94552_at | Cluster Incl AI839477 UI-M-AL0-abu-e-11-0-UI s2 Mus musculus cDNA, 3 end/clone = UI-M-AL0-abu-e-11-0-UI/ | P | 2693.4 | P | 3594.1 | P | 2843.6 | P |
| | | clone_end = 3/gb = AI839477/gi = 54773690/ug = Mm 29089/len = 474/STRA = for | | | | | | | |
| 11 | 94558_g_at | Cluster Incl AI850893 UI-M-BH0-ajt-g-07-0-UI s1 Mus musculus cDNA, 3 end/clone = UI-M-BH0-ajt-g-07-0-UI/clone_end = 3/ | P | 376 | P | 693.6 | P | 413.4 | P |
| | | gb = AI850893/gi = 5494799/ug = Mm.29105/len = 375/STRA = for | | | | | | | |
| 11 | 95053_s_at | Cluster Incl AA674669 vq02d04.r1 Mus musculus cDNA, 5 end/clone = IMAGE-1093063/clone_end = 5/gb = AA674669/ | P | 1526.5 | P | 1639.7 | P | 1812.1 | P |
| | | gi = 2651906/ug = Mm 29141/len = 414/STRA = for | | | | | | | |
| 11 | 95076_at | Cluster Incl AI848476 UI-M-AP1-agf-a-07-0-UI s2 Mus musculus cDNA, 3 end/clone = UI-M-AP1-agf-a-07-0-UI/ | P | 1541.1 | P | 1835 | P | 1837.8 | P |
| | | clone_end = 3/gb = AI848476/gi = 5492382/ug = Mm.29202/len = 304/STRA = for | | | | | | | |
| 11 | 95090_at | Cluster Incl AA688600 vu18f09 r1 Mus musculus cDNA, 5 end/clone = IMAGE-1181033/clone_end = 5/gb = AA688600/ | P | 1087.2 | P | 1410.3 | P | 1342.7 | P |
| | | gi = 2678029/ug = Mm 29269/len = 362/STRA = rev | | | | | | | |
| 11 | 95100_at | Cluster Incl AI847314 UI-M-AN1-afh-e-04-0-UI s1 Mus musculus cDNA, 3 end/clone = UI-M-AN1-afh-e-04-0-UI/ | P | 2003.7 | P | 3052.6 | P | 2502.7 | P |
| | | clone_end = 3/gb = AI847314/gi = 5491220/ug = Mm 29341/len = 379/STRA = for | | | | | | | |
| 11 | 95120_at | Cluster Incl AI837621. UI-M-AK0-adg-d-09-0-UI.s1 Mus musculus cDNA, 3 end/clone = UI-M-AK0-adg-d-09-0-UI/ | P | 212.1 | P | 325 | P | 179.4 | P |
| | | clone_end = 3/gb = AI837621/gi = 5471834/ug = Mm 29386/len = 473/STRA = for | | | | | | | |
| 11 | 95652_at | Cluster Incl AF110520 Mus musculus major histocompatibility complex region NG27, NG28, RPS28, NADH | P | 2212.2 | P | 3146.1 | P | 2456.3 | P |
| | | oxidoreductase, NG29, KIFC1, Fas-binding protein, BING1, tapasin, RalGDS-like, KE2, BING4, beta 1,3-galactosyl | | | | | | | |
| | | transferase, and RPS18 genes, complete cds, S/STRA = for | | | | | | | |
| 11 | 95662_at | Cluster Incl AW211667 uo83c04 y1 Mus musculus cDNA, 5 end/clone = IMAGE-2649126/clone_end = 5/gb = AW211667/ | P | 238.7 | P | 348.1 | P | 260.2 | P |
| | | gi = 6517563/ug = Mm 29562/len = 454/STRA = for | | | | | | | |
| 11 | 95674_r_at | Cluster Incl AI851985 UI-M-BH0-aiq-d-08-0-UI s1 Mus musculus cDNA, 3 end/clone = UI-M-BH0-aiq-d-08-0-UI/ | P | 1578 | P | 2399.7 | P | 1666.7 | P |
| | | clone_end = 3/gb = AI851985/gi = 5495891/ug = Mm 29586/len = 404/STRA = for | | | | | | | |
| 11 | 95689_at | Cluster Incl AI840995 UI-M-AK0-adk-g-06-0-UI s1 Mus musculus cDNA, 3 end/clone = UI-M-AK0-adk-g-06-0-UI/ | P | 796.8 | P | 1111 | P | 793.4 | P |
| | | clone_end = 3/gb = AI840995/gi = 5475208/ug = Mm 29650/len = 438/STRA = for | | | | | | | |
| 11 | 95715_at | Cluster Incl AW125739 UI-M-BH2 2-aqi-g-05-0-UI s1 Mus musculus cDNA, 3 end/clone = UI-M-BH2-agi-g-05-0-UI/ | P | 1711.8 | P | 3386.4 | P | 2541.4 | P |
| | | clone_end = 3/gb = AW125739/gi = 6101234/ug = Mm 29714/len = 321/STRA = for | | | | | | | |
| 11 | 96272_at | Cluster Incl AI848471 UI-M-AH1-ags-h-12-0-UI s1 Mus musculus cDNA, 3 end/clone = UI-M-AH1-ags-h-12-0-UI/ | P | 591.7 | P | 875.8 | P | 885.8 | P |
| | | clone_end = 3/gb = AI848471/gi = 5492377/ug = Mm 29855/len = 427/STRA = for | | | | | | | |
| 11 | 96284_at | Cluster Incl AW121446 UI-M-BH2.2-aon-d-06-0-UI s1 Mus musculus cDNA, 3 end/clone = UI-M-BH2 2-aon-d-06-0-UI/ | P | 728.4 | P | 1044.9 | P | 827.5 | P |
| | | clone_end = 3/gb = AW121446/gi = 6096779/ug = Mm 29873/len = 220/STRA = for | | | | | | | |
| 11 | 96285_at | Cluster Incl AJ001616 Mus musculus mRNA for myeloid associated differentiation protein/cds = (187,1077)/gb = AJ001616/ | P | 1576.8 | P | 1867.6 | P | 2140.9 | P |
| | | gi = 2463264/ug = Mm 29874/len = 2822/STRA = for | | | | | | | |
| 11 | 96320_at | Cluster Incl AW121862 UI-M-BH2.3-aoi-c-03-0-UI.s1 Mus musculus cDNA, 3 end/clone = UI-M-BH2-aoi-c-03-0-UI/ | P | 627.4 | P | 656.8 | P | 537.7 | P |
| | | clone_end = 3/gb = AW121862/gi = 6097236/ug = Mm 29937/len = 495/STRA = for | | | | | | | |
| 11 | 96345_at | Cluster Incl AI837012 UI-M-AJ0-aaz-h-08-0-UI s1 Mus musculus cDNA, 3 end/clone = UI-M-AJ0-aaz-h-08-0-UI/ | P | 576.7 | P | 524.7 | P | 535.8 | P |
| | | clone_end = 3/gb = AI837012/gi = 5471225/ug = Mm 29995/len = 460/STRA = for | | | | | | | |
| 11 | 96346_at | Cluster Incl AI854020 UI-M-BH0-ajm-b-01-0-UI s1 Mus musculus cDNA, 3 end/clone = UI-M-BH0-ajm-b-01-0-UI/ | P | 1019.4 | P | 1123.7 | P | 710.9 | P |
| | | clone_end = 3/gb = AI854020/gi = 5497926/ug = Mm 29996/len = 262/STRA = for | | | | | | | |
| 11 | 96869_at | Cluster Incl AW124839 UI-M-BH2.1-apk-f-02-0-UI s1 Mus musculus cDNA, 3 end/clone = UI-M-BH2 1-apk-f-02-0-UI/ | P | 2523.5 | P | 3189.7 | P | 3498.5 | P |
| | | clone_end = 3/gb = AW124839/gi = 6100369/ug = Mm 30064/len = 465/STRA = for | | | | | | | |
| 11 | 96887_at | Cluster Incl Y08702 M musculus mRNA for neuronal protein 15.6/cds = (51,452)/gb = Y08702/gi = 1771305/Mm.30084/ | P | 1714.4 | P | 1851.8 | P | 1770.1 | P |
| | | len = 554/STRA = for | | | | | | | |
| 11 | 96888_at | Cluster Incl AI839814 UI-M-AN0-acq-b-07-0-UI s1 Mus musculus cDNA, 3 end/clone = UI-M-AN0-acq-b-07-0-UI/ | P | 2947.8 | P | 3343.9 | P | 3435.4 | P |
| | | clone_end = 3/gb = AI839814/gi = 5473992/ug = Mm 30085/len = 342/STRA = for | | | | | | | |
| 11 | 96890_at | Cluster Incl AW060971 UI-M-BH1-anv-h-09-0-UI s1 Mus musculus cDNA, 3 end/clone = UI-M-BH1-anv-h-09-0-UI/ | P | 367 | P | 283.7 | P | 326.9 | P |
| | | clone_end = 3/gb = AW060971/gi = 6008722/ug = Mm 30092/len = 539/STRA = for | | | | | | | |
| 11 | 96911_at | Cluster Incl U34960 Mouse G protein beta 2 subunit mRNA, complete cds/cds = (0,1022)/gb = U34960/gi = 984550/ | P | 1828.1 | P | 1875.8 | P | 2343.5 | P |
| | | ug = Mm 30141/len = 1023/STRA = for | | | | | | | |
| 11 | 96915_f_at | Cluster Incl AA879764 vv98d04 r1 Mus musculus cDNA, 5 end/clone = IMAGE-1230439/clone_end = 5/gb = AA879764/ | P | 1786 | P | 2190.2 | P | 1787.5 | P |
| | | gi = 2988747/ug = Mm 30150/len = 357/STRA = for | | | | | | | |
| 11 | 96948_at | Cluster Incl AI845337 UI-M-BG0-ahx-b-12-0-UI.s1 Mus musculus cDNA, 3 end/clone = UI-M-BG0-ahx-b-12-0-UI/ | P | 677.7 | P | 744 | P | 718.9 | P |
| | | clone_end = 3/gb = AI845337/gi = 5489243/ug = Mm 30204/len = 210/STRA = for | | | | | | | |
| 11 | 97446_at | Cluster Incl AW125010 UI-M-BH2.1-apv-g-03-0-UI s1 Mus musculus cDNA, 3 end/clone = UI-M-BH2 1-apv-g-03-0-UI/ | P | 241.3 | P | 332.1 | P | 252.3 | P |
| | | clone_end = 3/gb = AW125010/gi = 6100540/ug = Mm 30243/len = 428/STRA = for | | | | | | | |
| 11 | 97452_at | Cluster Incl AI853078 UI-M-BH0-ajf-a-06-0-UI.s1 Mus musculus cDNA, 3 end/clone = UI-M-BH0-ajf-a-06-0-UI/ | P | 665.4 | P | 1151.4 | P | 768.6 | P |
| | | clone_end = 3/gb = AI853078/gi = 5496984/ug = Mm 30255/len = 429/STRA = rev | | | | | | | |
| 11 | 97464_at | Cluster Incl AI844416 UI-M-AL1-ahp-g-01-0-UI s1 Mus musculus cDNA, 3 end/clone = UI-M-AL1-ahp-g-01-0-UI/ | P | 1027.6 | P | 1092.5 | P | 1032.4 | P |
| | | clone_end = 3/gb = AI844416/gi = 5488322/ug = Mm 3035/len = 432/STRA = for | | | | | | | |
| 11 | 98120_at | Cluster Incl AI844807 UI-M-AH1-agx-b-03-0-UI s1 Mus musculus cDNA, 3 end/clone = UI-M-AH1-agx-b-03-0-UI/ | P | 675.4 | P | 584.2 | P | 634.6 | P |
| | | clone_end = 3/gb = AI844807/gi = 5488713/ug = Mm 34951/len = 517/STRA = for | | | | | | | |
| 11 | 98147_at | Cluster Incl AC002397: Mouse chromosome 6 BAC-284H12 (Research Genetics mouse BAC library) complete sequence/ | P | 731.4 | P | 830.7 | P | 950.5 | P |
| | | cds = (52,2628)/gb = AC002397/gi = 3287367/ug = Mm 3571/len = 3176/STRA = rev | | | | | | | |
| 11 | 98149_s_at | Cluster Incl AW046496: UI-M-BH1-akz-d-01-0-UI.s1 Mus musculus cDNA, 3 end/clone = UI-M-BH1-akz-d-01-0-UI/ | P | 456.8 | P | 495.1 | P | 359.3 | P |
| | | clone _end = 3/gb = AW046496/gi = 5907025/ug = Mm.3572/len = 413/STRA = for | | | | | | | |
| 11 | 98516_at | Cluster Incl AI853136: UI-M-BH0-ajf-f-11-0-UI s1 Mus musculus cDNA, 3 end/clone = UI-M-BH0-ajf-f-11-0-UI/clone_end = 3/ | P | 809.8 | P | 1065.1 | P | 857.4 | P |
| | | gb = AI853136/gi = 5497042/ug = Mm.35790/len = 291/STRA = for | | | | | | | |
| 11 | 98535_at | Cluster Incl AF076156 Calechol-O-methyltransferase/cds = (163,960)/gb = AF076156/gi = 3493252/ug = Mm 35853/ | P | 677.7 | P | 903.8 | P | 994.6 | P |
| | | len = 1255/STRA = for | | | | | | | |
| 11 | 98544_at | Cluster Incl U53514 Mus musculus guanylate kinase (gmk) mRNA, complete cds/cds = (24,620)/gb = U53514/gi = 1408589/ | P | 519.4 | P | 643.2 | P | 520.1 | P |
| | | ug = Mm 3624/len = 663/STRA = for | | | | | | | |
| 11 | 98627_at | Cluster Incl X81580: Insulin-like growth factor binding protein 2/cds = (71,988)/gb = X81580 gi = 550378/ug = Mm 38748/ | P | 459.8 | P | 469.2 | P | 367.1 | P |
| | | len = 1310/STRA = for | | | | | | | |
| 11 | 99115_at | Cluster Incl AW046350: UI-M-BH1-ali-a-01-0-UI s1 Mus musculus cDNA, 3 end/clone = UI-M-BH1-ali-a-01-0-UI/ | P | 2038.2 | P | 2948.8 | P | 2471.4 | P |
| | | clone_end = 3/gb = AW046350/gi = 5906879/ug = Mm 400/len = 253/STRA = for | | | | | | | |
| 11 | 99164_at | Cluster Incl AI844560 UI-M-AJ1-ahg-c-05-0-UI s2 Mus musculus cDNA, 3 end/clone = UI-M-AJ1-ahg-c-05-0-UI/ | P | 429.4 | P | 488.7 | P | 351.5 | P |
| | | clone_end = 3/gb = AI844560/gi = 5488466/ug = Mm 41680/len = 357/STRA = for | | | | | | | |
| 11 | 99175_at | Cluster Incl AI843396 UI-M-AQ1-adz-g-07-0-UI s1 Mus musculus cDNA, 3 end/clone = UI -M-AQ1-adz-g-07-0-UI/ | P | 631.5 | P | 606.8 | P | 552.2 | P |
| | | clone_end = 3/gb = AI843396/gi = 5477609/ug = Mm 41780/len = 289/STRA = for | | | | | | | |
| 11 | 99584-at | Cluster Incl D14883 CD82 anligen/cds = (102,902)/gb = D14883/gi = 984036/ug = Mm 4261/len = 1657/STRA = for | P | 376.8 | P | 615.5 | P | 406.9 | P |
| 11 | 99599_s_at | Cluster Incl AW210320: ul53e09 y1 Mus musculus cDNA, 5 end/clone = IMAGE-2102056/clone_end = 5/gb = AW210320/ | P | 1688.5 | P | 1952.1 | P | 2006.4 | P |
| | | gi = 6516260/ug = Mm 42855/len = 477/STRA = for | | | | | | | |
| 11 | 99629_at | Cluster Incl AW060710: UI-M-BH1-anl-g-04-0-UI s1 Mus musculus cDNA, 3 end/clone = UI -M-BH1-anl-g-04-0-UI/ | P | 973.3 | P | 1061.6 | P | 1202.5 | P |
| | | clone_end = 3/gb = AW060710/gi = 6008461/ug = Mm 4337/len = 535/STRA = for | | | | | | | |
| 11 | 99651_at | Cluster Incl AI849549 UI-M-AH1-agr-a-10-0-UI s1 Mus musculus cDNA, 3 end/clone = UI-M-AH1-agr-a-10-0-UI/ | P | 407.5 | P | 501.9 | P | 471 | P |
| | | clone end = 3/gb = AI849549/gi = 5493455/ug = Mm 43737/len = 279/STRA = for | | | | | | | |
| 11 | 99665_at | Cluster Incl U05252 Mus musculus nuclear matrix attachment DNA-binding protein SATB1 mRNA, complete cds/ | P | 469.7 | P | 658.3 | P | 687.8 | P |
| | | cds = (198,2492)/gb = U05252/gi = 453370/ug = Mm 4381/len = 2922/STRA = for | | | | | | | |
| 11 | 100039_at | Cluster Incl AW125880 UI-M-BH2 2-aqk-g-03-0-UI s1 Mus musculus cDNA, 3 end/clone = UI-M-BH2 2-aqk-g-03-0-UI/ | P | 580.2 | P | 408.3 | P | 303.2 | P |
| | | clone_end = 3/gb = AW125880/gi = 6101410/ug = Mm 44220/len = 490/STRA = for | | | | | | | |
| 11 | 100153_at | Cluster Incl X15052 Neural cell adhesion molecule/cds = (0,1246)/gb = X15052/gi = 53349/ug = Mm 4974/len = 4552/ | P | 457.5 | P | 579.5 | P | 741.7 | P |
| | | STRA = for | | | | | | | |
| 11 | 100538_at | Cluster Incl M35725 Mouse Cu-Zn superoxide dismutase mRNA, complete cds/cds(4,468)/gb = M35725/gi = 192929/ | P | 1466.8 | P | 1522.5 | P | 1482.6 | P |
| | | ug = Mm 5274/len = 469/STRA = for | | | | | | | |
| 11 | 100540_at | Cluster Incl M63848: Mouse leukotnene A-4 hydrolase mRNA, complete cds/cds = (80,1915)/gb = M63848/gi = 198883/ | P | 769.1 | P | 1112.9 | P | 1201.5 | P |
| | | ug-Mm 533/len = 2039/STRA = for | | | | | | | |
| 11 | 100576_at | Cluster Incl U57746: Mus musculus platelet-activating, factor acetylhydrolase isoform lb gamma subunit (PAF-AH gamma) | P | 353.4 | P | 458.9 | P | 582.6 | P |
| | | mRNA, complete cds/cds = (129,627)/gb = U57746/gi = 1373360/ug = Mm 597/len = 872/STRA = for | | | | | | | |
| 11 | 100629_at | Cluster Incl U24428 Mus musculus mu-class glutathione s-transferase (mGSTM5) mRNA, complete cds/cds = (96,770)/ | P | 377.8 | P | 572.6 | P | 587.6 | P |
| | | gb = U24428/gi = 1002385/ug = Mm.667/len = 907/STRA = for | | | | | | | |
| 11 | 101000_at | Cluster Incl D78643 Mouse seizure-related mRNA, partial cds/cds = (0,422)/gb = D78643/gi = 1304167/ug = Mm 675/ | P | 482.2 | P | 539 | P | 617.7 | P |
| | | len = 1492/STRA = for | | | | | | | |
| 11 | 101007_at | Cluster Incl AI845732 UI-M-AQ1-aeb-b-04-0-UI s1 Mus musculus cDNA, 3 end/clone = UI-M-AQ1-aeb-b-04-0-UI/ | P | 953.7 | P | 1290 | P | 1411.6 | P |
| | | clone_end = 3/gb = AI845732/gi = 5489638/ug = Mm 6797/len = 184/STRA = for | | | | | | | |
| 11 | 101013_at | Cluster Incl U52623 Mus musculus omithine decarboxylase anlizyme gene, complete cds/cds = (0,683)/gb = U52823/ | P | 3101.7 | P | 4328.8 | P | 4040.1 | P |
| | | gi = 1924975/ug = Mm 683/len = 684/STRA = for | | | | | | | |
| 11 | 101042_f_at | Cluster Incl U51014 Peptidase 4/cds = (54,1535)/gb = U51014/gi = 1236705/ug = Mm 69751/len = 1876/STRA = for | P | 421.8 | P | 454.4 | P | 417.3 | P |
| 11 | 101053_at | Cluster Incl AW046847: UI-M-BH1-alz-h-03-0-UI s1 Mus musculus cDNA, 3 end/clone = UI-M-BH1-alz-h-03-0-UI/ | P | 569.6 | P | 629.4 | P | 640.8 | P |
| | | clone_end = 3/gb = AW046847/gi = 5907376/ug = Mm 7039/len = 242/STRA = for | | | | | | | |
| 11 | 101059_at | Cluster Incl D76440 Necdin/cds = (86,1063)/gb = D76440/gi = 1040690/ug = Mm 7089/len = 1608/STRA = for | P | 608.1 | P | 580.7 | P | 511.5 | P |
| 11 | 101072_at | Cluster Incl AW124250 UI-M-BH2.1-aph-f-11-0-UI s1 Mus musculus cDNA, 3 end/clone = UI-M-BH2.1-aph-f-11-0-UI/ | P | 1935.3 | P | 2461.3 | P | 2209.5 | P |
| | | clone_end = 3/gb = AW124250/gi = 6099780/ug = Mm 7215/len = 267/STRA = for | | | | | | | |
| 11 | 101080_at | Cluster Incl AB009993 Mus musculus mRNA for collagen a1(V), complete cds/cds = (131,5647)/gb = AB009993/ | P | 228.3 | P | 300.5 | P | 584.4 | P |
| | | gi = 3219171/ug = Mm 7281/len = 6156/STRA = for | | | | | | | |
| 11 | 101495_at | Cluster Incl X59047 M musculus MD3 mRNA/cds = UNKNOWN/gb = X59047/gi = 53037/ug = Mm 806/len = 1234/STRA = for | P | 1188 | P | 1519.6 | P | 1519.8 | P |
| 11 | 101589_at | Cluster Incl X12944 Mouse mRNA for HMG-17 chromosomal protein/cds = (39,311)/gb = X12944/gi = 51336/ug = Mm 911/ | P | 1585.7 | P | 2519.9 | P | 1494.1 | P |
| | | len = 1113/STRA = for | | | | | | | |
| 11 | 101976_at | Cluster Incl AF071314: Mus musculus COP9 complex subunit 4 (COPS4) mRNA, complete cds/cds = (37,1257)/ | P | 383.8 | P | 474.5 | P | 442.3 | P |
| | | gb = AF071314/gi = 3309169/ug = Mm 957/len = 1620/STRA = for | | | | | | | |
| 11 | 102003_at | Cluster Incl AI842159 UI-M-AN1-afj-f-10-0-UI.s1 Mus musculus cDNA, 3 end/clone = UI-M-AN1-afj-f-10-0-UI/clone_end = 3/ | P | 345 | P | 480.1 | P | 371.2 | P |
| | | gb = AI842159/gi = 5476372/ug = Mm 99132/len = 277/STRA = for | | | | | | | |
| 11 | 103332_at | Cluster Incl AI197507 ud56b04 r1 Mus musculus cDNA, 5 end/clone = IMAGE-1449871/clone_end = 5/gb = AI197507/ | P | 92.9 | P | 135.8 | P | 114.4 | P |
| | | gi = 3750113/ug = Mm 18062/len = 436/STRA = for | | | | | | | |
| 11 | 103591_at | Cluster Incl D49439 Mouse mRNA for TFIID subunit p80, complete cds/cds = (10,2046)/gb = D49439/gi = 1372970/ | P | 202.4 | P | 255.7 | P | 198.5 | P |
| | | ug = Mm 1994/len = 2192/STRA = for | | | | | | | |
| 11 | 103642_at | Cluster Incl AB001927: Mus musculus mRNA for ras-GTPase-activating protein SH3-domain binding protein, complete cds/ | P | 1704.9 | P | 1917.7 | P | 1603.8 | P |
| | | cds = (68,1465)/gb = AB001927/gi = 1902906/Ug = Mm.2038/len = 2686/STRA = for | | | | | | | |
| 11 | 103656_at | Cluster Incl Y16518 Mus musculus mRNA for G protein-coupled receptor, P40GPRT/cds = (257,1456)/gb = Y16518/ | P | 662.4 | P | 649.2 | P | 571.7 | P |
| | | gi = 3492792/ug = Mm 20522/len = 1581/STRA = for | | | | | | | |
| 11 | 103817_at | Cluster Incl AJ006469 Mus musculus mRNA for cartilage-associated protein (CASP)/cds = (71,1273)/gb = AJ006469/ | P | 1207.9 | P | 1198.3 | P | 1175.1 | P |
| | | gi = 3687319/us = Mm.20904/len = 1685/STRA = for | | | | | | | |
| 11 | 103837_at | Cluster Incl AF092997: Mus musculus caspase-14 mRNA, complete cds/cds = (60,833)/gb = AF092997/gi = 3719426/ | P | 1818.8 | P | 1957.2 | P | 2685.8 | P |
| | | ug = Mm 20940/len = 2175/STRA = for | | | | | | | |
| 11 | 103858_at | Cluster Incl AW124023: UI-M-BH2.1-apn-b-12-0-UI.s1 Mus musculus cDNA, 3 end/clone = UI-M-BH2.1-apn-b-12-0-UI/ | P | 198.6 | P | 231.7 | P | 181.6 | P |
| | | clone_end = 3/gb = AW124023/gi = 6099553/ug = Mm.21096/len = 436/STRA = for | | | | | | | |
| 11 | 104118_at | Cluster Incl AW123781 UI-M-BH2 1-apm-c-11-0-UI s1 Mus musculus cDNA, 3 end/clone = UI-M-BH2.1-apm-c-11-0-UI/ | P | 435.2 | P | 526.8 | P | 384 | P |
| | | clone_end = 3/gb = AW123781/gi = 6099311/ug = Mm.21972/len = 396/STRA = for | | | | | | | |
| 11 | 104143_at | Cluster Incl AI843212 UI-M-AK1-aet-f-07-0-UI s1 Mus musculus cDNA, 3 end/clone = UI-M-AK1-aet-f-07-0-UI/ | P | 498 | P | 642.5 | P | 426.3 | P |
| | | clone_end = 3/gb = AI843212/gi = 5477425/ug = Mm 22144/len = 368/STRA = for | | | | | | | |
| 11 | 104150_at | Cluster Incl AW121957 UI-M-BH2 3-aoi-h-06-0-UI s1 Mus musculus cDNA, 3 end/clone = UI-M-BH2 3-aoi-h-06-0-UI/ | P | 332.5 | P | 445.4 | P | 407.2 | P |
| | | clone_end = 3/gb = AW121957/gi = 6097308/ug = Mm 22189/len = 274/STRA = for | | | | | | | |
| 11 | 104376_at | Cluster Incl AF006602 Mus musculus histone deacetylase mHDA1 mRNA, complete cds/cds = (0,2975)/gb = AF006602/ | P | 740.2 | P | 854.6 | P | 1061.8 | P |
| | | gi = 4165859/ug = Mm 22665/len = 3081/STRA = for | | | | | | | |
| 11 | 104390_at | Cluster Incl AI836916: UI-M-AJ0-aav-c-12-0-UI.s1 Mus musculus cDNA, 3 end/clone = UI-M-AJ0-aav-c-12-0-UI/ | P | 550.7 | P | 658.8 | P | 540.5 | P |
| | | clone/end = 3/gb = AI836916/gi = 5471129/ug = Mm 22717/len = 489/STRA = for | | | | | | | |
| 11 | 104623_at | Cluster Incl X73360 M. musculus mESG mRNA/cds = (258,2573)/gb = X73360/gi = 313235/ug = Mm 24255/len = 2840/ | P | 451.8 | P | 858 | P | 498.3 | P |
| | | STRA = for | | | | | | | |
| 11 | 104624_at | Cluster Incl AI851539 UI-M-BH0-aki-c-09-0-UI.s1 Mus musculus cDNA, 3 end/clone = UI-M-BH0-aki-c-09-0-UI/ | P | 847.2 | P | 938.7 | P | 890.9 | P |
| | | clone_end = 3/gb = AI851539/gi = 5495445/ug = Mm.24260/len = 530/STRA = for | | | | | | | |
| 11 | 104715_at | Cluster Incl AI152779 ud91a09 r1 Mus musculus cDNA, 5 end/clone = IMAGE-1478200/clone_end = 5/gb = AI152779/ | P | 916.3 | P | 1249.7 | P | 1043.4 | P |
| | | gi = 3681248/ug = Mm.24496/len = 504/STRA = for | | | | | | | |
| 11 | 94330_at | Cluster Incl AA710564 vI43b11 r1 Mus musculus cDNA, 5 end/clone = IMAGE-1165821/clone_end = 5/gb = AA710564/ | P | 644.5 | P | 592.8 | P | 748.9 | P |
| | | gi = 2720482/ug = Mm.24887/len = 519/STRA = for | | | | | | | |
| 11 | 94384_at | Cluster Incl AI847926 UI-M-AI1-afl-c-08-0-UI.s1 Mus musculus cDNA, 3 end/clone = UI-M-AI1-afl-c-08-0-UI/clone_end = 3/ | P | 979.2 | P | 1323.4 | P | 1249.4 | P |
| | | gb = AI847926/gi = 5491827/ug = Mm 25279/len = 446/STRA = for | | | | | | | |
| 11 | 94410_f_at | Cluster Incl AA611413: vo51e11 r1 Mus musculus cDNA, 5 end/clone = IMAGE-1053452/clone_end = 5/gb = AA611413/ | P | 874 | P | 841.5 | P | 913.1 | P |
| | | gi = 2461492/ug = Mm 25941/len = 402/STRA = rev | | | | | | | |
| 11 | 94932_at | Cluster Incl M29464 Platelet derrved growth factor, alpha/cds = (62,652)/gb = M29464/gi = 200272/ug = Mm 2675/len = 906/ | P | 1571.3 | P | 1922.6 | P | 2197.2 | P |
| | | STRA = for | | | | | | | |
| 11 | 95629_at | Cluster Incl AI843679: UI-M-AK1-aer-c-01-0-UI s1 Mus musculus cDNA, 3 end/clone = UI-M-AK1-aer-c-01-0-UI/ | P | 646.6 | P | 519.9 | P | 548 | P |
| | | clone_end = 3/gb = AI843679/gi = 5477892/ug = Mm 28073/len = 184/STRA = for | | | | | | | |
| 11 | 95636_at | Cluster Incl AW123628 UI-M-BH2 1-api-e-09-0-UI s1 Mus musculus cDNA, 3 end/clone = UI-M-BH2 1-api-e-09-0-UI/ | P | 1126.6 | P | 1503.9 | P | 1212 | P |
| | | clone_end = 3/gb = AW123628/gi = 6099158/ug = Mm 28094/len = 416/STRA = for | | | | | | | |
| 11 | 96195_at | Cluster Incl U57686 Mus musculus p130Cas-related protein Sin mRNA, complete cds/cds = (57,1739)/gb = U57686/ | P | 602.2 | P | 589.8 | P | 701.9 | P |
| | | gi = 1373343/ug = Mm 28543/len = 2211/STRA = for | | | | | | | |
| 11 | 96236_at | Cluster Incl AW122965 UI-M-BH2 1-aoz-g-11-0-UI s2 Mus musculus cDNA, 3 end/clone = UI-M-BH2 1-aoz-g-11-0-UI/ | P | 536.9 | P | 531.5 | P | 601.9 | P |
| | | clone_end = 3/gb = AW122965/gi = 6098495/ug = Mm 28798/len = 620/STRA = for | | | | | | | |
| 11 | 96791 _at | Cluster Incl AW047875 UI-M-BH1-als-g-03-0-UI s1 Mus musculus cDNA, 3 end/clone = UI-M-BH1-als-g-03-0-UI/ | P | 841.9 | P | 1016.2 | P | 987.6 | P |
| | | clone end = 3/gb = AW047875/gi = 5908404/ug = Mm 29116/len = 324/STRA = for | | | | | | | |
| 11 | 97330_at | Cluster Incl AI840499 UI-M-AN0-aci-g-08-0-UI.s1 Mus musculus cDNA, 3 end/clone = UI-M-AN0-aci-g-08-0-UI/ | P | 1473.3 | P | 1600.9 | P | 1842.4 | P |
| | | clone_end = 3/gb = AI840499/gi = 5474712/ug = Mm 29543/len = 426/STRA = for | | | | | | | |
| 11 | 97386_at | Cluster Incl AI853294 UI-M-BH0-aji-f-03-0-UI s1 Mus musculus cDNA, 3 end/clone = UI-M-BH0-aji-f-03-0-UI/clone_end = 3/ | P | 701.6 | P | 943.6 | P | 565 | P |
| | | gb = AI853294/gi = 5497200/ug = Mm 29789/len = 413/STRA = for | | | | | | | |
| 11 | 97407_at | Cluster Incl AI848025 UI-M-AH1-agp-e-07-0-UI s1 Mus musculus cDNA, 3 end/clone = UI-M-AH1-agp-e-07-0-UI/ | P | 205 | P | 284.8 | P | 279.5 | P |
| | | clone_end = 3/gb = AI848025/gi = 5491931/ug = Mm 29926/len = 435/STRA = for | | | | | | | |
| 11 | 97415_at | Cluster Incl M89777 Mus musculus GTP-binding protein (Rab3D) mRNA, complete cds/cds = (43,702)/gb = M89777/ | P | 454.1 | P | 536.7 | P | 836.7 | P |
| | | gi = 200631/ug = Mm.29968/len = 703/STRA = for | | | | | | | |
| 11 | 97925_at | Cluster Incl AB026241.Mus musculus mRNA for casein kinase, complete cds/cds = (88,1338)/gb = AB028241/gi = 6526519/ | P | 237.9 | P | 372.2 | P | 344.2 | P |
| | | ug = Mm 30199/len = 1392/STRA = for | | | | | | | |
| 11 | 97953 g_at | Cluster Incl U39818 Tuberous sclerosis 2/cds = (90,5534)/gb = U39818/gi = 1236401/ug = Mm 30435/1en = 5637/STRA = for | P | 120.1 | P | 109.1 | P | 140.1 | P |
| 11 | 98440_at | Cluster Incl AA596710 vm60d08 r1 Mus musculus cDNA, 5 end/clone = IMAGE-1002639/clone_end = 5/gb = AA596710/ | P | 649.1 | P | 758.4 | P | 543.4 | P |
| | | gi = 2412145/ug = Mm 34497/len = 396/STRA = for | | | | | | | |
| 11 | 98447_at | Clusler Incl M62362.CCAAT/enhancer binding protein (C/EBP), alpha/cds = (0,1187)/gb = M62362/gi = 192381/ | P | 3097.7 | P | 3702.1 | P | 3623 | P |
| | | ug = Mm 34537/len = 1188/STRA = rev | | | | | | | |
| 11 | 98473_at | Cluster Incl AF032466: Mus musculus arginase II mRNA, complete cds/cds = (34,1098)/gb = AF032466/gi = 2642321/ | P | 253.1 | P | 266.3 | P | 251.3 | P |
| | | ug = Mm 3506/len = 1363/STRA = for | | | | | | | |
| 11 | 99926_at | Cluster Incl AB001489: Phosphatidylinositol glycan, class R/cds = (0,2315)/gb = AB001489/gi = 2804245/ug = Mm 4317/ | P | 452.9 | P | 614.5 | P | 467.6 | P |
| | | len = 3733/STRA = for | | | | | | | |
| 11 | 100410_at | Cluster Incl AW122781 UI-M-BH2 1-aoy-d-01-0-UI.s1 Mus musculus cDNA, 3 end/clone = UI-M-BH2 1-aoy-d-01-0-UI/ | P | 223.1 | P | 341.1 | P | 321.5 | P |
| | | clone_end = 3/gb = AW122781/gi = 6098311/ug = Mm 46641/len = 162/STRA = for | | | | | | | |
| 11 | 100890_at | Cluster Incl AI173038 uh16b06.r1 Mus musculus cDNA, 5 end/clone = IMAGE-1745555/clone_end = 5/gb = AI173038/ | P | 502.6 | P | 608.4 | P | 534.1 | P |
| | | gi = 3720618/ug = Mm 5458/len = 431/STRA = for | | | | | | | |
| 11 | 100927_at | Cluster Incl U28960 Phospholipid transfer protein/cds = (0,1475)/gb = U28960/gi = 902887/ug = Mm.6105/len = 1590/ | P | 253.3 | P | 298.3 | P | 391.6 | P |
| | | STRA = for | | | | | | | |
| 11 | 100948_at | Cluster Incl AW049351: UI-M-BH1-amr-h-10-0-UI s1 Mus musculus cDNA, 3 end/clone = UI-M-BH1-amr-h-10-0-UI/ | P | 844.1 | P | 1287.3 | P | 1110.6 | P |
| | | clone end = 3/gb = AW049351/gi = 5909880/ug = Mm 6403/len = 318/STRA = for | | | | | | | |
| 11 | 101430_at | Cluster Incl AW124153 UI-M-BH2.1-apr-10-0-UI s1 Mus musculus cDNA, 3 end/clone = UI-M-BH2 1-apr-f-10-0-UI/ | P | 623.7 | P | 1070 | P | 873.1 | P |
| | | clone_end = 3/gb = AW124153/gi = 6099683/ug = Mm 7556/len = 219/STRA = for | | | | | | | |
| 11 | 101441_i_at | Cluster Incl AF031127 Mus musculus inositol trisphosphate receptor type 2 (Itpr2) gene, partial cds/cds = (0,110)/ | P | 373.6 | P | 999.5 | P | 762.1 | P |
| | | gb = AF031127/gi = 2911341/ug = Mm 7800/len = 111/STRA = rev | | | | | | | |
| 11 | 101462_r_at | Cluster Incl U06944 Praja1/cds = (328,1602)/gb = U06944/gi = 2828789/ug = Mm 8211/len = 1978/STRA = for | P | 181.5 | P | 226.3 | P | 236.9 | P |
| 11 | 101930_at | Cluster Incl Y07688: M musculus mRNA for NfiX1-protein/cds = (316,1641)/gb = Y07688/gi = 1524166/ug = Mm 9394/ | P | 1515.1 | P | 2435.6 | P | 2265.1 | P |
| | | len = 1729/STRA = for | | | | | | | |
| 11 | 103485_at | Cluster Incl AA795415: vv06f02.r1 Mus musculus cDNA, 5 end/clone = IMAGE-1210875/clone_end = 5/gb = AA795415/ | P | 206.6 | P | 154.1 | P | 170.4 | P |
| | | gi = 2858370/ug = Mm.22139/len = 298/STRA = for | | | | | | | |
| 11 | 103921_i_at | Cluster Incl AI839690 UI-M-AN0-acp-e-04-0-UI.s1 Mus musculus cDNA, 3 end/clone = UI-M-AN0-acp-e-04-0-UI/ | P | 412 | P | 504.6 | P | 422.6 | P |
| | | clone_end = 3/gb = AI839690/gi = 5473903/ug = Mm 25034/len = 250/STRA = rev | | | | | | | |
| 11 | 103924_at | Cluster Incl AW048884 UI-M-BH1-amf-f-03-0-UI.s1 Mus musculus cDNA, 3 end/clone = UI-M-BH1-amf-f-03-0-UI/ | P | 499.1 | P | 637.9 | P | 646.5 | P |
| | | clone_end = 3/gb = AW048884/gi = 5909413/ug = Mm.25078/len = 251/STRA = rev | | | | | | | |
| 11 | 104016_at | Cluster Incl M91236: Gap junction membrane channel protein beta 5/cds = (120,935)/gb = M91236/gi = 192642/ | P | 351.9 | P | 667.2 | P | 534.4 | P |
| | | ug = Mm 26859/len = 1272/STRA = for | | | | | | | |
| 11 | 104025_at | Cluster Incl AW047185 UI-M-BH1-amb-a-12-0-UI s1 Mus musculus cDNA, 3 end/clone = UI-M-BH1-amb-a-12-0-UI/ | P | 483 | P | 534.9 | P | 685.1 | P |
| | | clone_end = 3/gb = AW047185/gi = 5907714/ug = Mm.26995/len = 416/STRA = for | | | | | | | |
| 11 | 104207_at | Cluster Incl AI430272 mf46f12 y1 Mus musculus cDNA, 5 end/clone = IMAGE-408143/clone_end = 5/gb = AI430272/ | P | 170.2 | P | 342.3 | P | 272.9 | P |
| | | gi = 4276108/ug = Mm 27654/len = 426/STRA = for | | | | | | | |
| 11 | 104232_at | Cluster Incl X63099 Gap junction membrane channel protein beta 3/cds = (119,931)/gb = X63099/gi = 50598/ug = Mm 28078/ | P | 699.6 | P | 1084.1 | P | 953 | P |
| | | len = 1636/STRA = for | | | | | | | |
| 11 | 104255_at | Cluster Incl AA795285 vv03h09 r1 Mus musculus cDNA, 5 end/clone = IMAGE-1210625/clone_end = 5/gb = AA795285/ | P | 213.3 | P | 332.4 | P | 313.9 | P |
| | | gi = 2858240/ug = Mm 28387/len = 297/STRA = rev | | | | | | | |
| 11 | 104400_at | Cluster Incl AF076956 Mus musculus prenylaied SNARE protein Ykt6 (Ykt6) mRNA, complete cds/cds = (15,611)/ | P | 91.1 | P | 154.4 | P | 163.9 | P |
| | | gb = AF076956/gi = 3328384/ug = Mm 28680/len = 643/STRA = for | | | | | | | |
| 11 | 104434_at | Cluster Incl AW048552 UI-M-BH1-alx-d-07-0-UI s1 Mus musculus cDNA, 3 end/clone = UI-M-BH1-alx-d-07-0-UI/ | P | 596.1 | P | 799.5 | P | 696.2 | P |
| | | clone_end = 3/gb = AW048552/gi = 5909081/ug = Mm 29241/len = 473/STRA = for | | | | | | | |
| 11 | 92310_at | Cluster Incl M96163 Mus musculus (clone 2) serum inducible kinase (SNK) mRNA, mRNA sequence/cds = UNKNOWN/ | P | 815.8 | P | 1043.9 | P | 890.4 | P |
| | | gb = M96163/gi = 200999/ug = Mm 380/len = 2772/STRA = for | | | | | | | |
| 11 | 92517_at | Cluster Incl AB026583 Mus musculus mRNA for Scr3, complete cds/cds = (34,1104)/gb = AB026583/gi = 4730922/ | P | 54.1 | P | 116 | P | 164.1 | P |
| | | ug = Mm 42247/len = 1354/STRA = for | | | | | | | |
| 11 | 92726_at | Cluster Incl AJ010605 SRY-box containing gene 6/cds = (0,931)/gb = AJ010605/gi = 3702611/ug = Mm 4656/len = 3371/ | P | 84.1 | P | 128.6 | P | −16.8 | P |
| | | STRA = for | | | | | | | |
| 11 | 93437_f _at | Cluster Incl AI850509 UI-M-BG1-aih-h-10-0-UI s1 Mus musculus cDNA, 3 end/clone = UI-M-BG1-aih-h-10-0-UI/ | P | 126.6 | P | 173.7 | P | 94.7 | P |
| | | clone_end = 3/gb = AI850509/gi = 5494415/ug = Mm 65832/len = 453/STRA = for | | | | | | | |
| 11 | 93467_at | Cluster Incl AI852734. UI-M-BH0-aji-h-02-0-UI s1 Mus musculus cDNA, 3 end/clone = UI-M-BH0-aji-h-02-0-UI/clone_end = 3/ | P | 339.4 | P | 367.9 | P | 367.4 | P |
| | | gb = AI852734/gi = 5496640/ug = Mm 6926/len = 348/STRA = for | | | | | | | |
| 11 | 93697_at | Cluster Incl U63387 Chromobox homolog 4 (Drosophila Pc class)/cds = (132,1787)/gb = U63387/gi = 2781391/ug = Mm 8556/ | P | 262.6 | P | 411.5 | P | 449 | P |
| | | len = 2542/STRA = for | | | | | | | |
| 11 | 94789_r_at | Cluster Incl X04663: Mouse mRNA for beta-tubulin (isotype Mbeta 5)/cds = (78,1412)/gb = X04663/gi = 55047/ug = Mm. 1703/ | P | 1926.3 | P | 2730.5 | P | 2624.4 | P |
| | | len = 1591/STRA = for | | | | | | | |
| 11 | 95952_at | Cluster Incl AW124781: UI-M-BH2.1-aqa-h-01-0-UI s1 Mus musculus cDNA, 3 end/clone = UI-M-BH2 1-aqa-h-01-0-UI/ | P | 265.8 | P | 393.6 | P | 311 | P |
| | | clone_end = 3/gb = AW124781/gi = 6100311/ug = Mm 24921/len = 422/STRA = rev | | | | | | | |
| 11 | 99872_s_at | Cluster Incl L39879: Ferritin light chain 1/cds = (205,756)/gb = L39879/gi = 666913/ug = Mm 7500/len = 920/STRA = for | P | 3450.2 | P | 3678.4 | P | 4318 | P |
| 11 | 100331_g_at | Cluster Incl AF032714 Mus musculus type II peroxiredoxin protein 2 (PrxII-2) pseudogene, complete sequence/ | P | 2976.7 | P | 4380.9 | P | 4489.4 | P |
| | | cds = (0,596)/gb = AF032714/gi = 3603231/ug = Mm 87831/len = 597/STRA = for | | | | | | | |
| 11 | 100342_l_at | Cluster Incl M28729: Tubulin alpha 1/cds = (0,596)/gb = M28729/gi = 202222/ug = Mm 88110/len = 786/STRA = for | P | 3503.7 | P | 5617.2 | P | 4950.3 | P |
| 11 | 95232_at | Cluster Incl AB009392 Mus musculus mRNA for protein L, partial cds/cds = (0,600)/gb = AB009392/gi = 2723394/ | P | 1342 | P | 2003.1 | P | 1830.6 | P |
| | | ug = Mm 9043/len = 958/STRA = for | | | | | | | |
| 11 | 95795_at | Cluster Incl U96810 Mus musculus Supt4h2 gene, complete cds/cds = (0,353)/gb = U96810/gi = 3779195/ug = Mm 87825/ | P | 560.8 | P | 609.1 | P | 753.1 | P |
| | | len = 354/STRA = for | | | | | | | |
| 11 | 96426_at | Cluster Incl U38967 Prothymosin beta 4/cds = (0,134)/gb = U38967/gi = 1072099/ug = Mm 22566/len = 542/STRA = for | P | 2658.6 | P | 2978.9 | P | 2649.5 | P |
| 11 | 101123_at | Cluster Incl U76253: Integral membrane protein 2 B/cds = (145,945)/gb = U76253/gi = 3746126/ug = Mm 4266/len = 1790/ | P | 2418.5 | P | 2130.4 | P | 2513.8 | P |
| | | STRA = for | | | | | | | |
| 11 | 101607_at | Cluster Incl C80068 C80068 Mus musculus cDNA, 3 end/clone = J0076B01/clone_end = 3/gb = C80068/gi = 2520398/ | P | 206.2 | P | 342.5 | P | 290.1 | P |
| | | ug = Mm 5905/len = 287/STRA = rev | | | | | | | |
| 11 | 102134_f_at | Cluster Incl AI461702 vw57d12.x1 Mus musculus cDNA, 3 end/clone = Image-1247927/clone_end = 3/gb = AI461702/ | P | 659.3 | P | 794.1 | P | 745.4 | P |
| | | gi = 4315732/ug = Mm.43556/len = 470/STRA = rev | | | | | | | |
| 11 | 92610_at | Cluster Incl M21332 Mouse MHC class III RD gene (H2-d and H2-Sk haplotypes), complete cds/cds = (0,1127)/gb = M21332/ | P | 283.6 | P | 440.7 | P | 321.1 | P |
| | | gi = 199607/ug = Mm.10964/len = 1128/STRA = for | | | | | | | |
| 11 | 93300_at | Cluster Incl X57413 Transforming growth factor, beta 2/cds = (1217,2461)/gb = X57413/gi = 54772/ug = Mm 18213/len = 4267/ | P | 88.6 | P | 101.4 | A | 116.1 | P |
| | | STRA = rev | | | | | | | |
| 11 | 93598_at | Cluster Incl AI840437 UI-M-AN0-aci-a-04-0-UI s1 Mus musculus cDNA, 3 end/clone = UI-M-AN0-aci-04-0-UI/ | P | 94.5 | P | 112.1 | P | 88.9 | A |
| | | clone_end = 3/gb = AI840437/gi = 5474650/ug = Mm.20847/len = 287/STRA = for | | | | | | | |
| 11 | 93967_at | Cluster Incl AI850979 UI-M-BH0-akd-h-04-0-UI.s1 Mus musculus cDNA, 3 end/clone = UI-M-BH0-akd-h-04-0-UI/ | P | 645.9 | P | 839.1 | P | 749.4 | A |
| | | clone_end = 3/gb = AI850979/gi = 5494885/ug = Mm.21645/len = 216/STRA = for | | | | | | | |
| 11 | 94277_at | Cluster Incl U66257. Mus musculus thrombospodin 3 and metaxin genes, exon 1, partial cds, and intergenic region/ | P | 253.1 | P | 293.3 | P | 310.2 | P |
| | | cds = (0,81)/gb = U66257/gi = 1575552/ug = Mm 22508/len = 82/STRA = for | | | | | | | |
| 11 | 94807_at | Cluster Incl AI848354 UI-M-AH1-ago-h-07-0-UI s1 Mus musculus cDNA, 3 end/clone = UI-M-AH1-ago-h-07-0-UI/ | P | 824.1 | P | 938.3 | P | 828.1 | A |
| | | clone_end = 3/gb AI848354/gi = 5492300/ug = Mm 22679/len = 221/STRA = for | | | | | | | |
| 11 | 95512_at | Cluster Incl M60320 Protein-L-isoaspartate (D-aspartate) O-Methyltransferase 1/cds = (119,802)/gb = M60320/gi = 200254/ | P | 114.8 | P | 179.7 | P | 93.2 | M |
| | | ug = Mm 25293/len = 1580/STRA = for | | | | | | | |
| 11 | 97807_at | Cluster Incl AI838630 UI-M-AO0-aca-a-04-0-UI s1 Mus musculus cDNA, 3 end/clone = UI-M-AO0-aca-a-04-0-UI/ | P | 227.3 | P | 159.5 | P | 109.6 | A |
| | | clone_end = 3/gb = AI838630/gi = 5472843/ug = Mm.2815/len = 430/STRA = for | | | | | | | |
| 11 | 98938_at | Cluster Incl AI852184 UI-M-BH0-ajc-a-05-0-UI s1 Mus musculus cDNA, 3 end/clone = UI-M-BHo-ajc-a-05-0-UI/ | P | 320.7 | P | 410.7 | P | 323.8 | A |
| | | clone_end = 3/gb = AI852184/gi = 5496090/ug = Mm 28690/len = 258/STRA = for | | | | | | | |
| 11 | 96873_at | Cluster Incl AI843287: UI-M-AO1-aei-e-06-0-UI s1 Mus musculus cDNA, 3 end/clone = UI-M-AO1-aei-e-06-0-UI/ | A | 511.6 | P | 514.7 | P | 563.9 | P |
| | | clone_end = 3/gb = AI843287/gi = 5477500/ug = Mm 30069/len = 425/STRA = for | | | | | | | |
| 11 | 96939_at | Cluster Incl AI842649: UI-M-AQ1-aed-g-07-0-UI.s1 Mus musculus cDNA, 3 end/clone = UI-M-AQ1-aed-g-07-0-UI/ | P | 312.9 | P | 365.1 | P | 326.7 | P |
| | | clone_end = 3/gb = AI842649/gi = 5476862/ug = Mm.30188/len = 312/STRA = for | | | | | | | |
| 11 | 102327_at | Cluster Incl AF078705: Amine oxidase, copper containing 3/cds = (0,2297)/gb = AF078705/gi = 3603354/ug = Mm 10734/ | P | 357.7 | P | 400.2 | P | 249 | A |
| | | len = 2298/STRA = for | | | | | | | |
| 11 | 102783_at | Cluster Incl AI131744 ue24h11.x1 Mus musculus cDNA, 3 end/clone = IMAGE-1481349/clone_end = 3/gb-AI131744/ | P | 459.9 | A | 703.3 | P | 506.4 | P |
| | | gi = 3601760/ug = Mm 13773/len = 472/STRA = for | | | | | | | |
| 11 | 103001_at | Cluster Incl U43836 Mus musculus VEGF-related factor mvrf186 precursor mRNA, complete cds/cds = (192,815)/ | P | 278.3 | P | 324.5 | P | 333.2 | A |
| | | gb = U43836/gi = 1703480/ug = Mm 15607/len = 1236/STRA = for | | | | | | | |
| 11 | 104742_at | Cluster Incl AA919832: vy18b09 r1 Mus musculus cDNA, 5 end/clone = IMAGE-1295801/clone_end = 5/gb = AA919832/ | P | 288.6 | P | 348.1 | P | 336.2 | P |
| | | gi = 3066611/ug = Mm 24679/len = 474/STRA = for | | | | | | | |
| 11 | 97928_at | Cluster Incl AW045278 UI-M-BH1-akn-c-07-0-UI s1 Mus musculus cDNA, 3 end/clone = UI-M-BH1-akn-c-07-0-UI/ | P | 188.9 | P | 247.8 | P | 217.9 | P |
| | | clone_end = 3/gb = AW045278/gi = 5905807/ug = Mm 30232/len = 250/STRA = for | | | | | | | |
| 11 | 98004_at | Cluster Incl M63554: Mus musculus inhibitor protein of cAMP-dependent protein kinase, complete cds/cds = (29,259)/ | P | 48.8 | P | 78.8 | P | 78.5 | A |
| | | gb = M63554/gi = 200384/ug = Mm.3193/len = 2278/STRA = for | | | | | | | |
| 11 | 103755_at | Cluster Incl D89677 Mus musculus mRNA for Kryn, complete cds/cds = (1123,3087)/gb = D89677/gi = 1944454/ | A | 163.6 | P | 252.2 | P | 217.4 | P |
| | | ug = Mm 2454/len = 4936/STRA = for | | | | | | | |
| 11 | 104169_at | Cluster Incl D32167 Zinc finger protein of the cerebellum 1/cds = (530,1873)/gb = D32167/gi = 565654/ug = Mm 2719/ | P | 202.3 | P | 359.1 | P | 217 | P |
| | | len = 2947/STRA = for | | | | | | | |
| 11 | 92180_at | Cluster Incl AI851599 UI-M-BH0-ain-a-02-0-UI s1 Mus musculus cDNA, 3 end/clone = UI-M-BH0-ain-a-02-0-UI/ | P | 236.9 | P | 298.7 | P | 97.9 | P |
| | | clone_end = 3/gb = AI851599/gi = 5495505/ug = Mm 33796/len = 352/STRA = for | | | | | | | |
| 11 | 92322_at | Cluster Incl X94353 Cathelin-like protein/cds = (14,535)/gb = X94353/gi = 1177533/ug = Mm 3834/len = 593/STRA = for | P | 392.6 | P | 791.9 | P | 954 | P |
| 11 | 94801_at | Cluster Incl AI607332 ub76a01 x1 Mus musculus cDNA, 3 end/clone = IMAGE-1383624/clone_end = 3/gb = AI607332/ | A | 82.9 | P | 115.1 | P | 133.9 | P |
| | | gi = 4616499/ug = Mm 1823/len = 431/STRA = for | | | | | | | |
| 11 | AFFX- | X57349 M musculus mRNA for transferrin receptor (_5, _M, _3 represent transcript regions 5 prime, | P | 90.2 | P | 229.2 | P | 350.2 | A |
| | TransRecMur/ | Middle, and 3 prime respectively) | | | | | | | |
| | X57349_3_at | | | | | | | | |
| 11 | 93009_at | Cluster Incl J04696 Mouse glutathione S-transferase class mu (GST5-5) mRNA, complete cds/cds = (4,660)/gb = J04696/ | P | 276.6 | P | 380.3 | P | 407.3 | A |
| | | gi = 193549/ug = Mm 14601/len = 1014/STRA = for | | | | | | | |
| 11 | 94217_f_at | Cluster Incl AC002397 Mouse chromosome 6 BAC-284H12 (Research Genetics mouse BAC library) complete sequence/ | P | 220.3 | P | 275.4 | P | 221.2 | M |
| | | cds = (198,998)/gb = AC002397/gi = 3287367/ug = Mm 22228/len = 1155/STRA = for | | | | | | | |
| 11 | 95501_at | Cluster Incl AW122970 UI-M-BH2 1-aoz-h-06-0-UI s2 Mus musculus cDNA, 3 end/clone = UI-M-BH2.1-aoz-h-06-0-UI/ | P | 191.3 | P | 200.5 | P | 208.3 | A |
| | | clone_end = 3/gb = AW122970/gi = 6098500/ug = Mm 25157/len = 435/STRA = for | | | | | | | |
| 11 | 99085_at | Cluster Incl AI021421: ub12a08 r1 Mus musculus cDNA, 5 end/clone = IMAGE-1366742/clone_end = 5/gb = AI021421/ | P | 172.4 | P | 254.9 | P | 290.7 | P |
| | | gi = 3235757/ug = Mm 38976/len = 470/STRA = for | | | | | | | |
| 11 | 99111_at | Cluster Incl U09874 Mus musculus SKD3 mRNA, complete cds/cds = (45,2078)/gb = U09874/gi = 563128/ug = Mm 3990/ | P | 177.2 | P | 234.7 | P | 192.8 | A |
| | | len = 2138/STRA = rev | | | | | | | |
| 11 | 100109_s_at | Cluster Incl X03492 Keratin complex 1, acidic, gene 13/cds = (0,938)/gb = X03492/gi = 52782/ug = Mm 4646/len = 1204/ | A | 172.4 | P | 264.3 | A | 239.1 | P |
| | | STRA = for | | | | | | | |
| 11 | 102022_at | Cluster Incl AW124555 UI-M-BH2 1-apt-b-05-0-UI s1 Mus musculus cDNA, 3 end/clone = UI-M-BH2 1-apt-b-05-0-UI/ | P | 180.2 | P | 199.9 | P | 192.5 | P |
| | | clone_end = 3/gb = AW124555/gi = 6100085/ug = Mm.10093/len = 547/STRA = for | | | | | | | |
| 11 | 104135_at | Cluster Incl AW045474 UI-M-BH1-akr-c-02-0-UI s1 Mus musculus cDNA, 3 end/clone = UI-M-BH1-akr-c-02-0-UI/ | P | 64.1 | P | 102.5 | P | 33.6 | P |
| | | clone_end = 3/gb = AW045474/gi = 5905968/ug = Mm 22085/len = 318/STRA = for | | | | | | | |
| 11 | 94983_at | Cluster Incl AI851614 UI-M-BH0-ain-b-07-0-UI s1 Mus musculus cDNA, 3 end/clone = UI-M-BH0-ain-b-07-0-UI/ | P | 249.7 | P | 270.1 | P | 245.8 | A |
| | | clone_end = 3/gb = AI851614/gi = 5495520/ug = Mm 27282/len = 425/STRA = for | | | | | | | |
| 11 | 97995_at | Cluster Incl X61385 Transcription factor 7, T-cell specific/cds = (189,1100)/gb = X61385/gi = 54760/ug = Mm.31630/len = 1790/ | P | 106 | P | 118.3 | M | 139.9 | P |
| | | STRA = for | | | | | | | |
| 11 | 102952_g_at | Cluster Incl AJ224738 Mus musculus mRNA for death adaptor molecule (Raidd/Cradd), clone 6-1/cds = (118,717)/ | A | 127.6 | P | 146.7 | P | 134.6 | P |
| | | gb = AJ224738/gi = 3929835/ug = Mm 17493/len = 1649/STRA = for | | | | | | | |
| 11 | 93431_at | Cluster Incl Z38015 Dystrophia myotonica kinase, B15/cds = (143,2038)/gb = Z38015/gi = 563525/ug = Mm 6529/len = 2755/ | P | 472.3 | P | 469.8 | P | 417.9 | A |
| | | STRA = for | | | | | | | |
| 11 | 93587_at | Cluster Incl L13129 Annexin VII/cds = (42,1433)/gb = L13129/gi = 293293/ug = Mm.20794/len = 2636/STRA = for | A | 85.8 | P | 193.6 | P | 134.2 | A |
| 11 | 95502_at | Cluster Incl AI840267: UI-M-AH0-act-e-08-0-UI s1 Mus musculus cDNA, 3 end/clone = UI-M-AH0-act-e-08-0-UI/ | P | 166.4 | P | 260.2 | P | 268.8 | P |
| | | clone_end = 3/gb = AI840267/gi = 5474480/ug = Mm.25161/len = 358/STRA = for | | | | | | | |
| 11 | 97886_at | Cluster Incl AI530375 ui92c09 y1 Mus musculus cDNA, 5 end/clone = IMAGE-1889872/clone_end = 5/gb = AI530375/ | P | 102.4 | A | 277.9 | P | 212.4 | P |
| | | gi = 4444510/ug = Mm 28393/len = 474/STRA = for | | | | | | | |
| 11 | 102848_f_at | Cluster Incl AI840577 UI-M-AM0-adp-h-10-0-U1.s1 Mus musculus cDNA, 3 end/clone = UI-M-AM0-adp-h-10-0-UI/ | A | 87.9 | P | 125 | A | 289.3 | P |
| | | clone_end = 3/gb = AI840577/gi = 5474790/ug = Mm 14808/len = 432/STRA = for | | | | | | | |
| 11 | 94403_at | Cluster Incl AW121330 UI-M-BH2 2-aom-b-01-0-UI s1 Mus musculus cDNA, 3 end/clone = UI-M-BH2 2-aom-b-01-0-UI/ | P | 177.6 | P | 216.3 | P | 285.8 | P |
| | | clone_end = 3/gb = AW121330/gi = 6096663/ug = Mm 25799/len = 555/STRA = for | | | | | | | |
| 11 | 96240_at | Cluster Incl AI851309 UI-M-BH0-akf-h-03-0-UI s1 Mus musculus cDNA, 3 end/clone = UI-M-BH0-akf-h-03-0-UI/ | A | 76.8 | P | 129.9 | P | 183.8 | P |
| | | clone_end = 3/gb = AI851309/gi = 5495215/ug = Mm 28819/len = 207/STRA = for | | | | | | | |
| 11 | 99490_at | Cluster Incl AW050348 UI-M-BH1-ang-d-06-0-UI s1 Mus musculus cDNA, 3 end/clone = UI-M-BH1-ang-d-06-0-UI/ | P | 120 | P | 176.2 | P | 108 | A |
| | | clone_end = 3/gb = AW050348/gi = 5910877/ug = Mm 41531/len = 493/STRA = for | | | | | | | |
| 11 | 102824_at | Cluster Incl AF031035: Mus musculus stanniocalcin (Stc2) mRNA, complete cds/cds = (199,1089)/gb = AF031035/ | P | 111.6 | P | 156.6 | P | 176.9 | A |
| | | gi = 4104012/ug = Mm 12904/len = 1793/STRA = for | | | | | | | |
| 11 | 101350_g_at | Cluster Incl U73170: Polo-like kinase, pseudogene 1/cds = (0,1788)/gb = U73170/gi = 2580507/ug = Mm 57017/len = 1789/ | P | 338.1 | P | 479.9 | P | 318.1 | M |
| | | STRA = for | | | | | | | |
| 11 | 101182_at | Cluster Incl X95255 GLI-Kruppel family member GLI3/cds = (171,4981)/gb = X95255/gi = 1419013/ug = Mm 5098/len = 5113/ | P | 12.1 | A | 58.6 | P | 53.5 | A |
| | | STRA = for | | | | | | | |
| 11 | 94518_at | Cluster Incl AW121749 UI-M-BH2 3-aoe-c-12-0-UI s1 Mus musculus cDNA, 3 end/clone = UI-M-BH2 3-aoe-c-12-0-UI/ | A | 407.7 | P | 511.7 | A | 367.4 | P |
| | | clone_end = 3/gb = AW121749/gi = 6097084/ug = Mm.28999/len = 313/STRA = for | | | | | | | |
| 11 | 98590_at | Cluster Incl D89571 Mus musculus mRNA for ryudocan core protein, complete cds/cds = (24,620)/gb = D89571/gi = 2373476/ | A | 82.8 | P | 261.6 | P | 142.7 | P |
| | | ug = Mm 3815/len = 2456/STRA = for | | | | | | | |
| 11 | 100539_at | Cluster Incl AI841279: UI-M-AM0-adu-e-08-0-UI.s1 Mus musculus cDNA, 3 end/clone = UI-M-AM0-adu-e-08-0-UI/ | P | 151 | M | 256.1 | P | 187.6 | A |
| | | clone_end = 3/gb = AI841279/gi = 5475492/ug = Mm.5293/len = 439/STRA = for | | | | | | | |
| 11 | 103671_at | Cluster Incl AF061972 Mus musculus Tat-Interacting protein TIP30 mRNA, complete cds/cds = (191,919)/gb = AF061972/ | P | 71.5 | P | 89 | P | 100.7 | A |
| | | gi = 3820524/ug = Mm 20801/len = 1178/STRA = for | | | | | | | |
| 11 | 95536_at | Cluster Incl AB025015: Mus musculus mRNA for elongin A, complete cds/cds = (8,2329)/gb = AB025015/gi = 6009623/ | P | 221.6 | P | 446.9 | A | 315.3 | P |
| | | ug = Mm 27663/len = 2537/STRA = for | | | | | | | |
| 11 | 99933_at | Cluster Incl D26107: Mouse mRNA for poliovirus receptor homolog protein soluble form, complete cds/cds = (0,1592)/ | P | 113.9 | A | 196.8 | P | 225.4 | P |
| | | gb = D26107/gi = 475017/ug = Mm.4341/len = 2443/STRA = for | | | | | | | |
| 11 | 102268_at | Cluster Incl AW123015 UI-M-BH2 1-apb-e-05-0-UI s1 Mus musculus cDNA, 3 end/clone = UI-M-BH2.1-apb-e-05-0-UI/ | P | 334 | P | 441.6 | P | 465 | A |
| | | clone_end = 3/gb = AW123015/gi = 6098545/ug = Mm 1116/len = 449/STRA = for | | | | | | | |
| 11 | 102643_at | Cluster Incl M93148 Homeo box A2/cds = (308,1426)/gb = M93148/gi = 193910/ug = Mm 131/len = 1460/STRA = for | P | 152.6 | P | 233.2 | M | 189.8 | A |
| 11 | 102943_at | Cluster Incl AI099344 ue37d12 y1 Mus musculus cDNA, 5 end/clone = IMAGE-1482551/clone_end = 5/gb = AI099344/ | P | 64.9 | A | 222.1 | A | 153.2 | P |
| | | gi = 3448869/ug = Mm 17231/len = 364/STRA = for | | | | | | | |
| 11 | 103933_at | Cluster Incl U61183: Yolk sac gene 2/cds = (12,1637)/gb = U61183/gi = 1628564/ug = Mm 2517/len = 2106/STRA = for | A | −9.6 | P | 15.8 | P | 34.7 | P |
| 11 | 102137_f_at | Cluster Incl AI845856 UI-M-AO1-aeg-e-12-0-UI s1 Mus musculus cDNA, 3 end/clone = UI-M-AO1-aeg-e-12-0-UI/ | A | 795.2 | A | 978.5 | P | 908.2 | P |
| | | clone_end = 3/gb = AI845856/gi = 5489762/ug = Mm 43942/len = 309/STRA = rev | | | | | | | |
| 11 | 95061_at | Cluster Incl AI838996 UI-M-AN0-acm-b-03-0-UI s1 Mus musculus cDNA, 3 end/clone = UI-M-AN0-acm-b-03-0-UI/ | P | 84.8 | A | 75.6 | M | 59.7 | P |
| | | clone_end = 3/gb = AI638996/gi = 5473209/ug = Mm.29162/len = 417/STRA = for | | | | | | | |
| 11 | 94372_at | Cluster Incl D49956 Mouse mRNA lor 8-oxo-dGTPase, complete cds/cds = (98,568)/gb = D49956/gi = 1065597/ | P | 226.3 | A | 319.5 | P | 243.2 | M |
| | | ug = Mm.2539/len = 939/STRA = for | | | | | | | |
| 11 | 96833_at | Cluster Incl AW048468 UI-M-BH1-alj-b-03-0-UI s1 Mus musculus cDNA, 3 end/clone = UI-M-BH1-alj-b-03-0-UI/ | A | 232.7 | A | 327 | P | 349.7 | A |
| | | clone_end = 3/gb = AW048468/gi = 5908997/ug = Mm.29420/len = 333/STRA = for | | | | | | | |
| 11 | 101382_at | Cluster Incl AF020198 Mus musculus transcription factor PBX2 (PBX2) mRNA, complete cds/cds = (85,1377)/ | P | 211.9 | A | 267.4 | A | 453.4 | A |
| | | gb = AF020198/gi = 2432012/ug = Mm 7103/len = 2805/STRA = for | | | | | | | |
| 11 | 101891_at | Cluster Incl Y09517. M musculus mRNA for 17-beta-hydroxysteroid dehydrogenase type II/cds = (94,1239)/gb = Y09517/ | P | 114.7 | P | 211.8 | A | 222.3 | P |
| | | gi = 2198500/ug = Mm 8877/len = 1342/STRA = for | | | | | | | |
| 11 | 92441_at | Cluster Incl Y10007 Fibroblast activation protein/cds = (170,2455)/gb = Y10007/gi = 1743329/ug = Mm 41816/len = 2715/ | A | 74.9 | P | 40.4 | M | 81.2 | A |
| | | STRA = for | | | | | | | |
| 11 | 93952_r_at | Cluster Incl AA409629 EST01370 Mus musculus cDNA, 3 end/clone = c0009A09/clone_end = 3/gb = AA409629/ | P | −2.4 | A | 87.5 | P | 58.1 | A |
| | | gi = 2066285/ug = Mm.94218/len = 305/STRA = rev | | | | | | | |
| 11 | 102094_f_at | Cluster Incl AI841270 UI-M-AM0-adu-d-10-0-UI s1 Mus musculus cDNA, 3 end/clone = UI-M-AM0-adu-d-10-0-UI/ | P | 451.5 | P | 532.1 | A | 444.5 | P |
| | | clone_end = 3/gb = AI841270/gi = 5475483/ug = Mm 42353/len = 210/STRA = rev | | | | | | | |
| 11 | 101980_at | Cluster Incl J03750 Mouse single stranded DNA binding protein p9 mRNA, complete cds/cds = UNKNOWN/gb = J03750/ | A | 43.3 | P | 173 | A | 149.5 | A |
| | | gi = 192235/ug = Mm.966/len = 2777/STRA = for | | | | | | | |
| 11 | 103607_at | Cluster Incl AI882309 ub98g02 r1 Mus musculus cDNA, 5 end/clone = IMAGE-1396562/clone_end = 5/gb = AI882309/ | A | 156.2 | A | 173.4 | P | 177.6 | A |
| | | gi = 5567398/ug = Mm.20164/len = 487/STRA = for | | | | | | | |
| 11 | 94406_at | Cluster Incl AJ242864: Mus musculus mRNA 2 for phtf protein/cds = (561,2846)/gb = AJ242864/gi = 6523390/ug = Mm 25877/ | A | 146.3 | P | 204.9 | A | 217.5 | A |
| | | len = 3330/STRA = for | | | | | | | |
| 11 | 96206_at | Cluster Incl AI854506 UI-M-BH0-ajj-b-09-0-UI s1 Mus musculus cDNA, 3 end/clone = UI-M-BH0-ajj-b-09-0-UI/clone_end = 3/ | A | 372.6 | P | 587 | A | 462.1 | P |
| | | gb = AI854506/gi = 5498412/ug = Mm 28639/len = 413/STRA = for | | | | | | | |
| 11 | 97990_at | Cluster Incl D85923 Myosin heavy chain 11, smooth muscle/cds = (104,6022)/gb = D85923/gi = 1945077/ug = Mm 3153/ | A | 204.1 | P | 235.8 | M | 238.2 | A |
| | | len = 6175/STRA = rev | | | | | | | |
| 11 | 103708_at | Cluster Incl AI132207: ue28g02.x1 Mus musculus cDNA, 3 end/clone = IMAGE-1481714/clone_end = 3/gb = AI132207/ | A | 145.4 | P | 52.3 | M | 94.7 | A |
| | | gi = 3602223/ug = Mm 24090/len = 450/STRA = for | | | | | | | |
| 11 | 104395_at | Cluster Incl AW121826 UI-M-BH2.3-anx-e-08-0-UI s2 Mus musculus cDNA, 3 end/clone = UI-M-BH2 3-anx-e-08-0-UI/ | A | 314.7 | P | 444.8 | P | 347.7 | A |
| | | clone_end = 3/gb = AW121826/gi = 6097196/ug = Mm 28604/len = 413/STRA = for | | | | | | | |
| 11 | 104681_at | Cluster Incl U96963 Diaphanous homolog 1 (Drosophila)/cds = (93,3880)/gb = U96963/gi = 2114472/ug = Mm.3282/len = 4378/ | P | 136.6 | A | 376.3 | A | 271.2 | A |
| | | STRA = for | | | | | | | |
| 11 | 94140_at | Cluster Incl M59446 Scavenger receptor/cds = (0,20)/gb = M59446/gi = 192739/ug = Mm 1227/len = 413/STRA = for | P | 194.5 | P | 400.7 | P | 187.8 | M |
| 11 | 97184_at | Cluster Incl AI528219 uI94h04.y1 Mus musculus cDNA, 5 end/clone = IMAGE-1890103/clone_end = 5/gb = AI528219/ | A | 161.7 | A | 182.4 | A | 237.3 | A |
| | | gi = 4442354/ug = Mm 36077/len = 505/STRA = for | | | | | | | |
| 11 | 101196_at | Cluster Incl D50060. Proprotein convertase subtilisin/kexin type 6/cds = (0,2798)/gb = D50060/gi = 769700/ug = Mm 5182/ | A | 129.8 | M | 298.8 | A | 225.2 | P |
| | | len = 3063/STRA = for | | | | | | | |
| 11 | 102120_f_at | Cluster Incl C80148: C80148 Mus musculus cDNA, 3 end/clone = J0077C02/clone_end = 3/gb = C80148/gi = 2520478/ | M | 573.4 | P | 791.7 | P | 634.4 | A |
| | | ug = Mm 42674/len = 583/STRA = rev | | | | | | | |
| 12 | 92544_f_at | Cluster Incl AF055983. Mus musculus proteasome alpha7/C8 subunit mRNA, complete cds/cds = (42,809)/gb = AF055983/ | P | 2367.2 | P | 2478.4 | P | 1501.5 | P |
| | | gi = 3037090/ug = Mm 1007/len = 938/STRA = for | | | | | | | |
| 12 | 92562_at | Cluster Incl U70475: Nuclear, factor, erythroid derived 2, like 2/cds = (139,1932)/gb = U70475/gi = 1778293/ug = Mm 1025/ | P | 500.1 | P | 317.9 | P | 229.1 | P |
| | | len = 2380/STRA = for | | | | | | | |
| 12 | 92564_at | Cluster Incl AI891475 uI59a06 x1 Mus musculus cDNA, 3 end/clone = IMAGE-2123314/clone_end = 3/gb = AI891475/ | P | 260.4 | P | 162.8 | P | 183.7 | P |
| | | gi = 5597377/ug = Mm 10272/len = 493/STRA = for | | | | | | | |
| 12 | 92565_at | Cluster Incl AI847109 UI-M-AP1-agn-g-04-0-UI s1 Mus musculus cDNA, 3 end/clone = UI-M-AP1-agn-g-04-0-UI/ | P | 594.1 | P | 498.6 | P | 356.4 | P |
| | | clone_end = 3/gb = AI847109/gi = 5491015/ug = Mm 10278/len = 324/STRA = for | | | | | | | |
| 12 | 92569_f_at | Cluster Incl AF053232 Mus musculus SIK similar protein mRNA, complete cds/cds = (270,1691)/gb = AF053232/ | P | 456.1 | P | 396.3 | P | 299.7 | P |
| | | gi = 2996193/ug = Mm 10303/len = 1879/STRA = for | | | | | | | |
| 12 | 92596_at | Cluster Incl U97327. Mus musculus calcyclin binding protein (CACYBP) mRNA, partial cds/cds = (0,744)/gb = U97327/ | P | 461.4 | P | 405.5 | P | 101.6 | P |
| | | gi = 3138917/ug = Mm. 10702/len = 1404/STRA = for | | | | | | | |
| 12 | 92797_at | Cluster Incl AI840051: UI-M-AL0-abl-a-01-0-UI s1 Mus musculus cDNA, 3 end/clone = UI-M-AL0-abI-a-01-0-UI/ | P | 745.8 | P | 636.2 | P | 681.1 | P |
| | | clone_end = 3/gb = AI840051/gi = 5474264/ug = Mm 12665/len = 450/STRA = for | | | | | | | |
| 12 | 92831_at | Cluster Incl AI846308: UI-M-AK1-aez-g-10-0-UI s1 Mus musculus cDNA, 3 end/clone = UI-M-AK1-aez-g-10-0-UI/ | P | 569.8 | P | 530.7 | P | 484.5 | P |
| | | clone_end = 3/gb = AI846308/gi = 5490214/ug = Mm 12983/len = 263/STRA = for | | | | | | | |
| 12 | 93045_at | Cluster Incl L28836: Peroxisomal membrane protein, 70 kDa/cds = (0,1979)/gb = L28836/gi = 454834/ug = Mm.1519/ | P | 658.9 | P | 602.1 | P | 465.1 | P |
| | | len = 1980/STRA = for | | | | | | | |
| 12 | 93058_at | Cluster Incl AF026481 Mus musculus elF-1A (elF-1A) mRNA, complete cds/cds = (1172,1606)/gb = AF026481/gi = 3746339/ | P | 436.3 | P | 308.1 | P | 280 | P |
| | | ug = Mm 1540/len = 2663/STRA = for | | | | | | | |
| 12 | 93111_at | Cluster Incl D45836 Mouse mRNA for nuclear pore-targeting complex component of 97 kDa, complete cds/cds = (198,2828)/ | P | 887 | P | 713.7 | P | 613.4 | P |
| | | gb = D45836/gi = 871889/ug = Mm 16710/len = 3897/STRA = for | | | | | | | |
| 12 | 93120_f_at | Cluster Incl V00746 Mus musculus H-2K gene for MHC class I antigen H-2K (allele b), exons 1 to 3 and joined CDS/ | P | 2698.3 | P | 1612.9 | P | 1481 | P |
| | | cds = (0,1109)/gb = V00746/gi = 51154/ug = Mm 16771/len = 1110/STRA = rev | | | | | | | |
| 12 | 93277_at | Cluster Incl X53584 Mouse mRNA for HSP60 protein (clones 3T3-7, −9, and −M1)/cds = (0,1667)/gb = X53584/gi = 51451/ | P | 2610.3 | P | 2393 | P | 1851.4 | P |
| | | ug = Mm 1777/len = 2119/STRA = for | | | | | | | |
| 12 | 93310_at | Cluster Incl AI836423 UI-M-AI0-aak-b-03-0-UI s1 Mus musculus cDNA, 3 end/clone = UI-M-AI0-aak-b-03-0-UI/ | P | 2388.2 | P | 1874.8 | P | 1389.9 | P |
| | | clone_end = 3/gb = AI836423/gi = 5470636/ug = Mm 18472/len = 565/STRA = for | | | | | | | |
| 12 | 93547_at | Cluster Incl L03279 Core binding factor beta/cds = UNKNOWN/gb = L03279/gi = 200177/ug = Mm 2018/len = 2685/STRA = for | P | 2175.7 | P | 1796.8 | P | 1449.1 | P |
| 12 | 93600_at | Cluster Incl AJ011565 Mus musculus mRNA for leptin receptor gene-related protein/cds = (0,395)/gb = AJ011565 | P | 725.8 | P | 597.3 | P | 570.7 | P |
| | | gi = 3688429/ug = Mm 20879/len = 635/STRA = for | | | | | | | |
| 12 | 93727_at | Cluster Incl AB003502 Mus musculus mRNA for Guanine Nucleotide Regulatory Protein, partial cds/cds = (0,1652)/ | P | 616.5 | P | 637.5 | P | 361.7 | P |
| | | gb = AB003502/gi = 3461879/ug = Mm 20925/len = 2079/STRA = for | | | | | | | |
| 12 | 93729_at | Cluster Incl Y15197: Mus musculus mRNA for E-MAP-115 protein/cds = (0,2192)/gb = Y15197/gi = 3417385/ug = Mm 20928/ | P | 131.6 | P | 133.6 | P | 118.6 | P |
| | | len = 2893/STRA = for | | | | | | | |
| 12 | 93747_at | Cluster Incl AW122599 UI-M-BH2.2-aox-h-11-0-UI s1 Mus musculus cDNA, 3 end/clone = UI-M-BH2 2-aox-h-11-0-UI/ | P | 1076.8 | P | 1050.1 | P | 463.2 | P |
| | | clone_end = 3/gb = AW122599/gi = 6098125/ug = Mm 21092/len = 456/STRA = for | | | | | | | |
| 12 | 93826_at | Cluster Incl AI956230: ul73a07 y1 Mus musculus cDNA, 5 end/clone = IMAGE-2136180/clone_end = 5/gb = AI956230/ | P | 1031.7 | P | 880.5 | P | 817 | P |
| | | gi = 5748967/ug = Mm 21544/len = 513/STRA = for | | | | | | | |
| 12 | 93829_at | Cluster Incl AW107884: um18c08 x 1 Mus musculus cDNA, 3 end/clone = IMAGE-2192654/clone_end = 3/gb = AW107884/ | P | 1586.7 | P | 1696.9 | P | 1384.6 | P |
| | | gi = 6078684/ug = Mm 21556/len = 579/STRA = for | | | | | | | |
| 12 | 93832_at | Cluster Incl AI854541: UI-M-BH0-ajj-e-12-0-UI s1 Mus musculus cDNA, 3 end/clone = UI-M-BH0-ajj-o-12-0-UI/clone_end = 3/ | P | 567.3 | P | 633.7 | P | 561.4 | P |
| | | gb = AI854541/gi = 5498447/ug = Mm 21569/len = 395/STRA = for | | | | | | | |
| 12 | 93970_at | Cluster Incl AI843085: UI-M-AK1-aes-b-10-0-UI s1 Mus musculus cDNA, 3 end/clone = UI-M-AK1-aes-b-10-0-UI/ | P | 427.4 | P | 404.9 | P | 358.1 | P |
| | | clone_end = 3/gb = AI843085/gi = 5477298/ug = Mm 21659/len = 279/STRA = for | | | | | | | |
| 12 | 93976_at | Cluster Incl AI836686: UI-M-AP0-abh-h-07-0-UI s1 Mus musculus cDNA, 3 end/clone = UI-M-AP0-abh-h-07-0-UI/ | P | 750.5 | P | 550.9 | P | 539.4 | P |
| | | clone_end = 3/gb = AI836686/gi = 5470899/ug = Mm 21684/len = 371/STRA = for | | | | | | | |
| 12 | 94008_at | Cluster Incl AW045358: UI-M-BH1-ako-c-05-0-UI s1 Mus musculus cDNA, 3 end/clone = UI-M-BH1-oko-c-05-0-UI/ | P | 385.7 | P | 294.8 | P | 248.2 | P |
| | | clone_end = 3/gb = AW045358/gi = 5905887/ug = Mm 21811/len = 296/STRA = for | | | | | | | |
| 12 | 94014_at | Cluster Incl AA760073: vv69b08 r1 Mus musculus cDNA, 5 end/clone = IMAGE-1227639/clone_end = 5/gb = AA760073/ | P | 1021.2 | P | 875.1 | P | 682.3 | P |
| | | gi = 2807867/ug = Mm 2184/len = 404/STRA = for | | | | | | | |
| 12 | 94019_at | Cluster Incl AI852534 UI-M-BH0-alw-e-03-0-UI s1 Mus musculus cDNA, 3 end/clone = UI-M-BH0-aiw-e-03-0-UI/ | P | 928.5 | P | 644.9 | P | 454.6 | P |
| | | clone_end = 3/gb = AI852534/gi = 5496440/ug = Mm 21848/len = 265/STRA = for | | | | | | | |
| 12 | 94242_at | Cluster Incl AA881309: vx11d11.r1 Mus musculus cDNA, 5 end/clone = IMAGE-1264149/clone_end = 5/gb = AA881309/ | P | 1407.1 | P | 1044.4 | P | 859.1 | P |
| | | gi = 2990619/ug = Mm 22354/len = 461/STRA = for | | | | | | | |
| 12 | 94253_at | Cluster Incl AW061243 UI-M-BH1-amj-c-11-0-UI s2 Mus musculus cDNA, 3 end/clone = UI-M-BH1-amj-c-11-0-UI/ | P | 592.9 | P | 686.7 | P | 392.1 | P |
| | | clone_end = 3/gb = AW061243/gi = 6008994/ug = Mm 22399/len = 430/STRA = for | | | | | | | |
| 12 | 94281_at | Cluster Incl AW125138 UI-M-BH2 1-apz-c-01-0-UI s1 Mus musculus cDNA, 3 end/clone = UI-M-BH2 1-apz-c-01-0-UI/ | P | 456.2 | P | 433.2 | P | 314.2 | P |
| | | clone_end = 3/gb = AW125138/gi = 6100668/ug = Mm 22533/len = 352/STRA = for | | | | | | | |
| 12 | 94322_at | Cluster Incl D42048 Mouse mRNA for squalene epoxidase, complete cds/cds = (619,2337)/gb = D42048/gi = 1217592/ | P | 837.3 | P | 837.7 | P | 736.5 | P |
| | | ug = Mm.22663/len = 2611/STRA = for | | | | | | | |
| 12 | 94325_at | Cluster Incl AW124932 UI-M-BH2 1-apu-g-09-0-UI s1 Mus musculus cDNA, 3 end/clone = UI-M-BH2.1-apu-g-09-0-UI/ | P | 1731.3 | P | 1330.3 | P | 925 | P |
| | | clone_end = 3/gb = AW124932/gi = 6100462/ug = Mm 22675/len = 143/STRA = for | | | | | | | |
| 12 | 94814_at | Cluster Incl AI957190 ul76g02.x1 Mus musculus cDNA, 3 end/clone = IMAGE-2136530/clone_end = 3/gb = AI957190/ | P | 1577.8 | P | 1322 | P | 1350.5 | P |
| | | gi = 5749899/ug = Mm 22704/len = 499/STRA = for | | | | | | | |
| 12 | 94832_at | Cluster Incl U58105 Mus musculus Btk locus, alpha-D-galactosidase A (Ags), ribosomal protein (L44L), and Bruton a | P | 374.5 | P | 287.7 | P | 122.6 | P |
| | | tyrosine kinase (Btk) genes, complete cds/cds = (3,1352)/gb = U58105/gi = 1666699/ug = Mm 22757/len = 2150/STRA = for | | | | | | | |
| 12 | 94865_at | Cluster Incl AW050059 UI-M-BH1-amm-e-04-0-UI s1 Mus musculus cDNA, 3 end/clone = UI-M-BH1-amm-e-04-0-UI/ | P | 266.3 | P | 231 | P | 167.6 | P |
| | | clone_end = 3/gb = AW050059/gi = 5910588/ug = Mm 23744/len = 335/STRA = for | | | | | | | |
| 12 | 94903_at | Cluster Incl AI850801 UI-M-BG1-aii-f-04-0-UI s1 Mus musculus cDNA, 3 end/clone = UI-M-BG1-aii-f-04-0-UI/clone_end = 3/ | P | 1397.9 | P | 1269 | P | 937.9 | P |
| | | gb = AI850801/gi = 5494707/ug = Mm 24086/len = 547/STRA = for | | | | | | | |
| 12 | 95408_at | Cluster Incl AW124487: UI-M-BH2 1-ape-h-10-0-UI.s1 Mus musculus cDNA, 3 end/clone = UI-M-BH2.1-ape-h-10-0-UI/ | P | 1212.2 | P | 1035.8 | P | 833.1 | P |
| | | clone_end = 3/gb = AW124487/gi = 6099982/ug = Mm 24220/len = 214/STRA = for | | | | | | | |
| 12 | 95431_at | Cluster Incl AA623426: vn33a01.r1 Mus musculus cDNA, 5 end/clone = IMAGE-1022952/clone_end = 5/gb = AA623426/ | P | 130 | P | 94.3 | P | 56.5 | P |
| | | gi = 2527302/ug = Mm 24463/len = 303/STRA = for | | | | | | | |
| 12 | 95448_at | Cluster Incl AI839371 UI-M-AN0-acj-g-11-0-UI s1 Mus musculus cDNA, 3 end/clone = UI-M-AN0-acj-g-11-0-UI/ | P | 820.3 | P | 897.6 | P | 791.9 | P |
| | | clone_end = 3/gb = AI839371/gi = 5473584/ug = Mm 2462/len = 393/STRA = for | | | | | | | |
| 12 | 95467_at | Cluster Incl AW120746 UI-M-BH2.3-any-c-11-0-UI.s1 Mus musculus cDNA, 3 end/clone = UI-M-BH2 3-any-c-11-0-UI/ | P | 333.3 | P | 189.2 | P | 157.2 | P |
| | | clone_end = 3/gb = AW120746/gi = 6096079/ug = Mm 24763/len = 416/STRA = for | | | | | | | |
| 12 | 95508_at | Cluster Incl X61453: M musculus mRNA for H19 clone/cds = (0,1946)/gb = X61453/gi = 51135/ug = Mm 25203/len = 2092/ | P | 1893.3 | P | 1664.4 | P | 1269.8 | P |
| | | STRA = for | | | | | | | |
| 12 | 96010_at | Cluster Incl AW047023: UI-M-BH1-alp-h-11-0-UI s1 Mus musculus cDNA, 3 end/clone = UI-M-BH1-alp-h-11-0-UI/ | P | 793.4 | P | 709.2 | P | 643.1 | P |
| | | clone_end = 3/gb = AW047023/gi = 5907552/ug = Mm 25548/len = 399/STRA = for | | | | | | | |
| 12 | 96011_at | Cluster Incl AB009275 Mus musculus gene for matrin3, complete cds/cds = (0,920)/gb = AB009275/gi = 6497040/ | P | 283.9 | P | 200.6 | P | 186 | P |
| | | ug = Mm.25575/len = 921/STRA = for | | | | | | | |
| 12 | 96012_f_at | Cluster Incl AI835367: UI-M-AQ0-aah-h-02-0-UI.s1 Mus musculus cDNA, 3 end/clone = UI-M-AQ0-aah-h-02-0-UI/ | P | 2688 | P | 2872.3 | P | 2252.2 | P |
| | | clone_end = 3/gb = AI835367/gi = 5469580/ug = Mm.25575/len = 407/STRA = for | | | | | | | |
| 12 | 96013_l_at | Cluster Incl AI835367: UI-M-AQ0-aah-h-02-0-UI s1 Mus musculus cDNA, 3 end/clone = UI-M-AQ0-aah-h-02-0-UI/ | P | 332.5 | P | 298.5 | P | 312.9 | P |
| | | clone_end = 3/gb = AI835367/gi = 5469580/ug = Mm.25575/len = 407/STRA = for | | | | | | | |
| 12 | 96019_at | Cluster Incl AI843476: UI-M-AQ1-aec-g-07-0-UI s1 Mus musculus cDNA,3 end/clone = UI-M-AQ1-aoc-g-07-0-UI/ | P | 1668.9 | P | 1384.7 | P | 1518.6 | P |
| | | clone_end = 3/gb = AI843476/gi = 5477889/ug = Mm.256999/len = 339/STRA = for | | | | | | | |
| 12 | 96061_at | Cluster Incl AW060186: UI-M-BH1-amu-e-06-0-UI s1 Mus musculus cDNA, 3 end/clone = UI-M-BH1-amu-e-06-0-UI/ | P | 347.6 | P | 284.9 | P | 304.5 | P |
| | | clone_end = 3/gb = AW060186/gi = 6007952/ug = Mm 25699/len = 339/STRA = for | | | | | | | |
| 12 | 96098_at | Cluster Incl AI848416: UI-M-AH1-ags-b-11-0-UI s1 Mus musculus cDNA,3 end/clone = UI-M-AH1-ags-b-11-0-UI/ | P | 311.9 | P | 301.2 | P | 204.6 | P |
| | | clone_end = 3/gb = AI848416/gi = 5492322/ug = Mm 26775/len = 499/STRA = for | | | | | | | |
| 12 | 96628_at | Cluster Incl X54327: M musculus mRNA for glutamyl-tRNA synthetase/cds = UNKNOWN/gb = X54327/gi = 5804944/ | P | 435.3 | P | 380.3 | P | 257.7 | P |
| | | ug = Mm 27190/len = 3528/STRA = for | | | | | | | |
| 12 | 96647_at | Cluster Incl AA645440: vq66d12.s1 Mus musculus cDNA, 5 end/clone = IMAGE-1107287/clone_end = 5/gb = AA645440/ | P | 574.2 | P | 448.6 | P | 321.2 | P |
| | | gi = 2571869/ug = Mm 27293/len = 456/STRA = for | | | | | | | |
| 12 | 96695_at | Cluster Incl AF089812 Mus musculus ubiquitin-conjugating enzyme HR6A mRNA, complete cds/cds = (128,586)/ | P | 234.2 | P | 211 | P | 208.8 | P |
| | | gb = AF089812/gi = 3769548/ug = Mm 27541/len = 1647/STRA = for | | | | | | | |
| 12 | 97205_at | Cluster Incl AI848056 UI-M-AH1-agp-h-08-0-UI.s1 Mus musculus cDNA, 3 end/clone = UI-M-AH1-agp-h-08-0-UI/ | P | 869.7 | P | 842.1 | P | 815 | P |
| | | clone_end = 3/gb = AI848056/gi = 5491962/ug = Mm 27695/len = 369/STRA = for | | | | | | | |
| 12 | 97237_at | Cluster Incl AI837771 UI-M-AK0-adl-d-01-0-UI s1 Mus musculus cDNA, 3 end/clone = UI-M-AK0-adl-d-01-0-UI/ | P | 449.7 | P | 429.3 | P | 265 | P |
| | | clone_end = 3/gb = AI837771/gi = 5471989/ug = Mm 27831/len = 480/STRA = for | | | | | | | |
| 12 | 97274_at | Cluster Incl Y13071 Mus musculus mRNA for 26S proteasome non-ATPase subunit/cds = (236,1165)/gb = Y13071/ | P | 1239.9 | P | 1147.5 | P | 824.8 | P |
| | | gi = 2505939/ug = Mm 27933/len = 1479/STRA = for | | | | | | | |
| 12 | 97292_at | Cluster Incl AI845225 UI-M-BG0-aht-f-07-0-UI.s1 Mus musculus cDNA, 3 end/clone = UI-M-BG0-aht-f-07-0-UI/ | P | 1077 | P | 758.6 | P | 518.8 | P |
| | | clone_end = 3/gb = AI845225/gi = 5489131/ug = Mm 28026/len = 258/STRA = for | | | | | | | |
| 12 | 97305_at | Cluster Incl AW123267 UI-M-BH2 1-apg-e-11-0-UI s1 Mus musculus cDNA, 3 end/clone = UI-M-BH2.1-apg-e-11-0-UI/ | P | 383.6 | P | 417.8 | P | 327.9 | P |
| | | clone_end = 3/gb = AW123267/gi = 6098797/ug = Mm 28053/len = 147/STRA = for | | | | | | | |
| 12 | 98912_at | Cluster Incl AW047811 UI-M-BH1-als-a-04-0-UI s1 Mus musculus cDNA, 3 end/clone = UI-M-BH1-als-a-04-0-UI/ | P | 399.3 | P | 280.5 | P | 194.4 | P |
| | | clone_end = 3/gb = AW047811/gi = 5908340/ug = Mm 28600/len = 426/STRA = for | | | | | | | |
| 12 | 98948_at | Cluster Incl AI785289 uj39f05 x1 Mus musculus cDNA, 3 end/clone = IMAGE-1922337/clone_end = 3/gb = AI785289/ | P | 293.1 | P | 194.8 | P | 137 | P |
| | | gi = 5333005/ug = Mm 28726/len = 523/STRA = for | | | | | | | |
| 12 | 98956_at | Cluster/Incl AI844979 UI-M-BG0-ahu-c-07-0-UI s1 Mus musculus cDNA, 3 end/clone = UI-M-BG0-ahu-c-07-0-UI/ | P | 1358.3 | P | 1363.9 | P | 1003 | P |
| | | clone_end = 3/gb = AI844979/gi = 5488885/ug = Mm 28765/len = 245/STRA = for | | | | | | | |
| 12 | 94462_at | Cluster Incl AW120719 UI-M-BH2 3-any-a-06-0-UI s1 Mus musculus cDNA, 3 end/clone = UI-M-BH2 3-any-a-06-0-UI/ | P | 429.5 | P | 402.5 | P | 322.1 | P |
| | | clone_end = 3/gb = AW120719/gi = 6096052/ug = Mm 28839/len = 482/STRA = for | | | | | | | |
| 12 | 94471 _r_at | Cluster Incl AW045974 UI-M-BH1-alc-a-08-0-UI s1 Mus musculus cDNA, 3 end/clone = UI-M-BH1-alc-a-08-0-UI/ | P | 142.6 | P | 80 | P | 63.5 | P |
| | | clone_end = 3/gb = AW045974/gi = 5906503/ug = Mm 28851/len = 338/STRA = for | | | | | | | |
| 12 | 94488_at | Cluster Incl AI847041 UI-M-AP1-agi-h-03-0-UI s1 Mus musculus cDNA, 3 end/clone = UI-M-AP1-agj-h-03-0-UI/ | P | 311.8 | P | 282.9 | P | 229.2 | P |
| | | clone_end = 3/gb = AI847041/gi = 5490947/ug = Mm 28899/len = 318/STRA = for | | | | | | | |
| 12 | 95050_at | Cluster Incl AW124547 UI-M-BH2 1-apt-a-04-0-UI.s1 Mus musculus cDNA, 3 end/clone = UI-M-BH2 1-apl-a-04-0-UI/ | P | 516.4 | P | 451.2 | P | 349.7 | P |
| | | clone_end = 3/gb = AW124547/gi = 6100077/ug = Mm 29138/len = 468/STRA = rev | | | | | | | |
| 12 | 95059_{— at} | Cluster Incl AW047399 UI-M-BH1-ame-g-05-0-UI s1 Mus musculus cDNA, 3 end/clone = UI-M-BH1-ame-g-05-0-UI/ | P | 1316.4 | P | 1056.3 | P | 976.7 | P |
| | | clone_end = 3/gb = AW047399/gi = 5907928/ug = Mm 29159/len = 417/STRA = for | | | | | | | |
| 12 | 95062_at | Cluster Incl AB026997 Mus musculus CAST mRNA for calpastatin, complete cds/cds = (249,2615)/gb = AB026997/ | P | 499.3 | P | 479.7 | P | 374.2 | P |
| | | gi = 6006274/ug = Mm.29163/len = 2622/STRA = for | | | | | | | |
| 12 | 95158_at | Cluster IncI AF100956 Mus musculus major histocompatibility locus class II region, Fas-binding protein Daxx (DAXX) gene, | P | 687 | P | 816.5 | P | 528.9 | P |
| | | partial cds: Bing1 (BING1), lapasin (tapasin), RalGDS-like factor (RLF), KE2 (KE2), BING4 (BING4), beta1, 3-galactosyl | | | | | | | |
| | | transferase (be/STRA = for | | | | | | | |
| 12 | 95694_at | Cluster Incl X70956: M musculus T0P gene for topoisomerase I, exons 19-21/cds = (0,347)/gb = X70956/gi = 57932/ | P | 1170.5 | P | 1213.4 | P | 690.7 | P |
| | | ug = Mm 29663/len = 348/STRA = for | | | | | | | |
| 12 | 95746_at | Cluster Incl AW123765 UI-M-BH2 1-apm-b-07-0-UI.s1 Mus musculus cDNA, 3 end/clone = UI-M-BH2.1-apm-b-07-0-UI/ | P | 1628.1 | P | 1102 | P | 1036.7 | P |
| | | clone_end = 3/gb = AW123765/gi = 6099295/ug = Mm 29771/len = 402/STRA = for | | | | | | | |
| 12 | 96269_at | Cluster Incl AA716963 vu69f10.r1 Mus musculus cDNA, 5 end/clone = IMAGE-1196883/clone_end = 5/gb = AA716963/ | P | 522.7 | P | 601.3 | P | 344.3 | P |
| | | gi = 2729237/ug = Mm 29847/len = 273/STRA = for | | | | | | | |
| 12 | 96277_at | Cluster Incl AI850118 UI-M-BG1-aid-a-10-0-UI.s1 Mus musculus cDNA, 3 end/clone = UI-M-BG1-aid-a-10-0-UI/ | P | 386.8 | P | 387.2 | P | 401.2 | P |
| | | clone_end = 3/gb = AI850118/gi = 5494024/ug = Mm 29864/len = 311/STRA = for | | | | | | | |
| 12 | 96289_at | Cluster Incl AW061287: UI-M-BH1-anw-b-03-0-UI s1 Mus musculus cDNA, 3 end/clone = UI-M-BH1-anw-b-03-0-UI/ | P | 627 | P | 720 | P | 472.1 | P |
| | | clone_end = 3/gb = AW061287/gi = 6009038/ug = Mm 29895/len = 336/STRA = for | | | | | | | |
| 12 | 96331_at | Cluster Incl AI842754: UI-M-AP1-agi-a-10-0-UI.s1 Mus musculus cDNA, 3 end/clone = UI-M-AP1-agi-a-10-0-UI/ | P | 519.5 | P | 266.4 | P | 164.6 | P |
| | | clone_end = 3/gb = AI842754/gi = 5476967/ug = Mm 29971/len = 325/STRA = for | | | | | | | |
| 12 | 96354_at | Cluster Incl AI854802: UI-M-BH0-aka-f-12-0-UI s1 Mus musculus cDNA, 3 end/clone = UI-M-BH0-aka-f-12-0-UI/ | P | 941.6 | P | 778.4 | P | 613.8 | P |
| | | clone_end = 3/gb = AI854802/gi = 5498708/ug = Mm 30007/len = 496/STRA = for | | | | | | | |
| 12 | 96858_at | Cluster Incl AF100927 Mus musculus apoptosis-inducing factor AIF (Aif) mRNA, nuclear gene encoding mitochondrial | P | 570.9 | P | 492.8 | P | 380.5 | P |
| | | protein, complele cds/cds = (51,1889)/gb = AF100927/gi = 4323584/ug = Mm 30050/len = 1932/STRA = for | | | | | | | |
| 12 | 96874_g_at | Cluster Incl AI843287: UI-M-AO1-aei-e-06-0-UI.s1 Mus musculus cDNA, 3 end/clone = UI-M-AO1-ael-e-06-0-UI/ | P | 2006.2 | P | 1464 | P | 1095.7 | P |
| | | clone_end = 3/gb = AI843287/gi = 5477500/ug = Mm 30069/len = 425/STRA = for | | | | | | | |
| 12 | 96878_at | Cluster Incl AW048566: UI-M-BH1-alx-e-09-0-UI s1 Mus musculus cDNA, 3 end/clone = UI-M-BH1-aix-e-09-0-UI/ | P | 130.8 | P | 117.8 | P | 101.8 | P |
| | | clone_end = 3/gb = AW048566/gi = 5909095/ug = Mm 30073/len = 430/STRA = for | | | | | | | |
| 12 | 96882_at | Cluster Incl AI842936: UI-M-AO1-aem-d-02-0-UI.s1 Mus musculus cDNA, 3 end/clone = UI-M-AO1-aem-d-02-0-UI/ | P | 1599.4 | P | 1668.9 | P | 1654.5 | P |
| | | clone_end = 3/gb = AI842936/gi = 5477149/ug = Mm.30078/len = 238/STRA = for | | | | | | | |
| 12 | 96892_at | Cluster Incl AI836804 UI-M-AJ0-abc-h-09-0-UI s1 Mus musculus cDNA, 3 end/clone = UI-M-AJ0-abc-h-09-0-UI | P | 511.9 | P | 532.3 | P | 471.8 | P |
| | | clone_end = 3/gb = AI836804/gi = 5471017/ug = Mm 30097/len = 512/STRA = for | | | | | | | |
| 12 | 96896_at | Cluster Incl AW214159: uo40f07.x1 Mus musculus cDNA, 3 end/clone = IMAGE-2645029/clone_end = 3/gb = AW214159/ | P | 2019 | P | 1506.2 | P | 1127.2 | P |
| | | gi = 6520601/ug = Mm.30108/len = 217/STRA = for | | | | | | | |
| 12 | 97471_at | Cluster Incl AI848442 UI-M-AH1-ags-e-10-0-UI s1 Mus musculus cDNA, 3 end/clone = UI-M-AH1-ags-e-10-0-UI/ | P | 358.4 | P | 324 | P | 371.3 | P |
| | | clone_end = 3/gb = AI848442/gi = 5492348/ug = Mm 30547/len = 212/STRA = for | | | | | | | |
| 12 | 97505_at | Cluster Incl AI845999 UI-M-AP1-agi-e-02-0-UI s1 Mus musculus cDNA, 3 end/clone = UI-M-AP1-agi-e-02-0-UI/ | P | 867.6 | P | 1002.9 | P | 740.9 | P |
| | | clone_end = 3/gb = AI845999/gi = 5489905/ug = Mm.31435/len = 386/STRA = for | | | | | | | |
| 12 | 97511_at | Cluster Incl AI846600 UI-M-AQ1-aef-e-03-0-UI s1 Mus musculus cDNA, 3 end/clone = UI-M-AQ1-aef-e-03-0-UI/ | P | 727.1 | P | 765.2 | P | 395.9 | P |
| | | clone_end = 3/gb = AI846600/gi = 5490506/ug = Mm 31616/len = 436/STRA = for | | | | | | | |
| 12 | 97514_at | Cluster Incl AW046438: UI-M-BH1-akp-d-11-0-UI s1 Mus musculus cDNA, 3 end/clone = UI-M-BH1-akp-d-11-0-UI/ | P | 223.2 | P | 228.9 | P | 178.7 | P |
| | | clone_end = 3/gb = AW046438/gi = 5906967/ug = Mm 31946/len = 438/STRA = rev | | | | | | | |
| 12 | 97540_f_at | Cluster Incl M69069 Histocompatibility 2, D region locus 1/cds = (8,1042)/gb = M69069/gi = 199795/ug = Mm.33263/ | P | 2056.5 | P | 982 | P | 598.9 | P |
| | | len = 1379/STRA = rev | | | | | | | |
| 12 | 98132_at | Cluster Incl X01756 Cytochrome c, somatic/cds = (0,317)/gb = X01756/gi = 50618/ug = Mm 35389/len = 318/STRA = for | P | 731.3 | P | 479.9 | P | 552.7 | P |
| 12 | 98136_at | Cluster Incl AF031486 Mus musculus apermidine aminopropyltransferase (Mapmsy) mRNA, complete cds/cds = (76,1176)/ | P | 447.6 | P | 392.9 | P | 260.6 | P |
| | | gb = AF031486/gi = 2625113/ug = Mm 3553/len = 1663/STRA = for | | | | | | | |
| 12 | 98142_at | Cluster Incl AA930290 vs58d02 r1 Mus musculus cDNA, 5 end/clone = IMAGE-1150467/clone_end = 5/gb = AA930290/ | P | 240.5 | P | 210.7 | P | 218.2 | P |
| | | gi = 3079883/ug = Mm.35618/len = 285/STRA = for | | | | | | | |
| 12 | 98572_at | Cluster Incl AW122551 UI-M-BH2.2-aox-d-09-0-UI s1 Mus musculus cDNA, 3 end/clone = UI-M-BH2 2-aox-d-09-0-UI/ | P | 263.2 | P | 203.1 | P | 236 | P |
| | | clone_end = 3/gb = AW122551/gi = 6098075/ug = Mm 37516/len = 480/STRA = for | | | | | | | |
| 12 | 98594_at | Cluster Incl AW125453: UI-M-BH2.2-aqm-a-02-0-UI s1 Mus musculus cDNA, 3 end/clone = UI-M-BH2 2-aqm-a-02-0-UI/ | P | 343.4 | P | 284.7 | P | 160.6 | P |
| | | clone_end = 3/gb = AW125453/gi = 6100983/ug = Mm.38211/len = 290/STRA = for | | | | | | | |
| 12 | 99107_at | Cluster Incl M31680: Growth hormone receptor/cds = (98,1072)/gb = M31680/gi = 193506/ug = Mm 3986/len = 1150/ | P | 209.4 | P | 218 | P | 135.7 | P |
| | | STRA = for | | | | | | | |
| 12 | 99138_at | Cluster Incl AA756292 vv40e08 r1 Mus musculus cDNA, 5 end/clone = IMAGE-1224902/clone_end = 5/gb = AA756292/ | P | 303.4 | P | 284.2 | P | 235.4 | P |
| | | gi = 2803490/ug = Mm.41374/len = 369/STRA = rev | | | | | | | |
| 12 | 99143_at | Cluster Incl AA614914: vo31go1 r1 Mus musculus cDNA, 5 end/clone = IMAGE-1051536/clone_end = 5/gb = AA614914/ | P | 430.5 | P | 294.2 | P | 249.4 | P |
| | | gi = 2502142/ug = Mm.4142/len = 375/STRA = for | | | | | | | |
| 12 | 99180_at | Cluster Incl AI851237 UI-M-BH0-akf-a-05-0-UI s1 Mus musculus cDNA, 3 end/clone = UI-M-BH0-akf-a-05-0-UI/ | P | 762.9 | P | 584.5 | P | 734.5 | P |
| | | clone_end = 3/gb = AI851237/gi = 5495143/ug = Mm.41800/len = 421/STRA = for | | | | | | | |
| 12 | 99196_at | Cluster Incl AI848532 UI-M-AP1-agf-f-07-0-UI a2 Mus musculus cDNA, 3 end/clone = UI-M-AP1-agf-07-0-UI/ | P | 1454.9 | P | 1030.2 | P | 645.3 | P |
| | | clone_end = 3/gb = AI848532/gi = 5492438/ug = Mm 41939/len = 378/STRA = for | | | | | | | |
| 12 | 99579_at | Cluster Incl U59761: ATPase, Na+/K+beta 3 polypeptide/cds = (148,984)/gb = U59761/gi = 1762431/ug = Mm 424/len = 1842/ | P | 2904.9 | P | 2351.4 | P | 1618.7 | P |
| | | STRA = for | | | | | | | |
| 12 | 100088_at | Cluster Incl M27073 Protein phosphatase 1, catalytic sutunit, beta isoform/cds = (95,1078)/gb = M27073/gi = 471977/ | P | 1370.4 | P | 1328.5 | P | 926.6 | P |
| | | ug = Mm 4572/len = 2243/STRA = for | | | | | | | |
| 12 | 100144_at | Cluster Incl X07699 Nucleolin/cds = (0,2123)/gb = X07699/gi = 53453/ug = Mm 4907/len = 2124/STRA = for | P | 2960.6 | P | 3974.6 | P | 3294.6 | P |
| 12 | 100560_at | Cluster Incl U95116 Platelet-activating factor acetylhydrolase, isoform 1b, beta 1 subunit/cds = (859,2091)/gb = U95116/ | P | 404.1 | P | 322.3 | P | 295.5 | P |
| | | gi = 2072302/ug = Mm.56337/len = 5832/STRA = for | | | | | | | |
| 12 | 100568_at | Cluster Incl U90446: Mus musculus RNAse L Inhibitor (Mu-RLI) mRNA, complete cds/cds = (114,1913)/gb = U90446/ | P | 729.8 | P | 626.7 | P | 475.6 | P |
| | | gi = 3273416/ug = Mm.5831/len.= 3509/STRA = for | | | | | | | |
| 12 | 101019_at | Cluster Incl U74683 Cathepsin C/cds = (67,1455)/gb = U74683/gi = 2145015/ug = Mm 684/len = 1767/STRA = for | P | 1561.5 | P | 1520.5 | P | 639 | P |
| 12 | 101023_f_at | Cluster Incl AI843605 UI-M-AO1-aeh-h-06-0-UI s1 Mus musculus cDNA, 3 end/clone = UI-M-AO1-aeh-h-06-0-UI/ | P | 1161.1 | P | 1115.4 | P | 1068.3 | P |
| | | clone_end = 3/gb = AI843605/gi = 5477783/ug = Mm.6844/len = 379/STRA = for | | | | | | | |
| 12 | 101035_at | Cluster Incl U35846 Mus musculus mRNA, complete cds/cds = (85,1599)/gb = U35846/gi = 2623754/ug = Mm 692/len = 3715/ | P | 413.6 | P | 349.4 | P | 252.1 | P |
| | | STRA = for | | | | | | | |
| 12 | 101057_at | Cluster Incl AW121180 UI-M-BH2 3-aok-a-08-0-UI s1 Mus musculus cDNA, 3 end/clone = UI-M-BH2 3-aok-a-08-0-UI/ | P | 420.5 | P | 416.6 | P | 336 | P |
| | | clone_end = 3/gb = AW121180/gi = 6096513/ug = Mm 7066/len = 437/STRA = for | | | | | | | |
| 12 | 101094_at | Cluster Incl AI836820 UI-M-AJ0-abd-c-01-0-UI s1 Mus musculus cDNA, 3 end/clone = UI-M-AJ0-abd-c-01-0-UI/ | P | 666.8 | P | 547.2 | P | 301.4 | P |
| | | clone_end = 3/gb = AI836820/gi = 5471033/ug = Mm.7381/1en = 357/STRA = for | | | | | | | |
| 12 | 101097_at | Cluster Incl AW121176 UI-M-BH2 3-aok-a-03-0-UI.s1 Mus musculus cDNA, 3 end/clone = UI-M-BH2 3-aok-a-03-0-UI/ | P | 327.6 | P | 333 | P | 304.7 | P |
| | | clone_end = 3/gb = AW121176/gi = 6096509/ug = Mm.74084/len = 435/STRA = for | | | | | | | |
| 12 | 101476_at | Cluster Incl U93050 Mus musculus poly(A) binding protein II (mPABII) gene, complete cds/cds = (0,908)/gb = U93050/ | P | 1071.8 | P | 776.1 | P | 790 | P |
| | | gi = 2351845/ug = Mm 7723/len = 909/STRA = for | | | | | | | |
| 12 | 101489_at | Cluster Incl D12780 Mouse mRNA for S-adenosylmethionine decarboxylase, complete cds/cds = (326,1330)/gb = D12780/ | P | 765.1 | P | 663.4 | P | 572.8 | P |
| | | gi = 220330/ug = Mm 7880/len = 1497/STRA = for | | | | | | | |
| 12 | 101518_at | Cluster Incl U38981 Mus musculus uterine mRNA, complete cds/cds = (205,603)/gb = U38981/gi = 1066284/ug = Mm 8486/ | P | 275.4 | P | 270.5 | P | 167.5 | P |
| | | len = 651/STRA = for | | | | | | | |
| 12 | 101523_at | Cluster Incl AW046194: UI-M-BH1-akw-f-03-0-UI.s1 Mus musculus cDNA, 3 end/clone-UI-M-BH1-akw-f-03-0-UI/ | P | 3610.6 | P | 3724.7 | P | 2197.9 | P |
| | | clone_end = 3/gb = AW046194/gi = 5906723/ug = Mm 86187/len = 380/STRA = for | | | | | | | |
| 12 | 101527_at | Cluster Incl M18209 Mouse transcription factor S-II, clone PSII-2/cds = (121,921)/gb = M18209/gi = 201936/ug = Mm 87235/ | P | 1005.4 | P | 879.6 | P | 792.6 | P |
| | | len = 2466/STRA = for | | | | | | | |
| 12 | 101528_at | Cluster Incl D00925 Mus musculus mRNA for transcription factor S-II-related proteins, complete cds/cds = UNKNOWN/ | P | 448.6 | P | 331.1 | P | 359 | P |
| | | gb = D00925/gi = 220590/ug = Mm.87235/len = 2257/STRA = for | | | | | | | |
| 12 | 101529_g_at | Cluster Incl D00925 Mus musculus mRNA for transcription factor S-II-related proteins, complete cds/cds = UNKNOWN/ | P | 378.9 | P | 236.6 | P | 254.8 | P |
| | | gb = D00925/gi = 220590/ug = Mm.87235/len = 2257/STRA = for | | | | | | | |
| 12 | 101548_at | Cluster Incl AI846345: UI-M-AN1-afa-c-08-0-UI s1 Mus musculus cDNA, 3 end/clone = UI-M-AN1-afa-c-08-0-UI/ | P | 166.7 | P | 162.5 | P | 153.5 | P |
| | | clone_end = 3/gb = AI846345/gi = 5490251/ug = Mm 88572/len = 394/STRA = rev | | | | | | | |
| 12 | 101955_at | Cluster Incl AJ002387 Mus musculus mRNA for BiP/cds = (11,1978)/gb = AJ002387/gi = 2598561/ug = Mm 918/len = 2282/ | P | 1553.1 | P | 1233.8 | P | 795.7 | P |
| | | STRA = for | | | | | | | |
| 12 | 102317_at | Cluster Incl AF061516 Mus musculus vesicle associated membrane protein 4 (Vamp4) mRNA, complete cds/cds = (77,502)/ | P | 413.7 | P | 356.5 | P | 325.8 | P |
| | | gb = AF061516/gi = 3108178/ug = Mm 10699/len = 819/STRA = for | | | | | | | |
| 12 | 102786_at | Cluster Incl AI849432: UI-M-AJ1-ahc-f-04-0-UI.s1 Mus musculus cDNA, 3 end/clone = UI-M-AJ1-ahc-f-04-0-UI/ | P | 433.3 | P | 335.4 | P | 317 | P |
| | | clone_end = 3/gb = AI849432/gi = 5493338/ug = Mm.13796/len = 291/STRA = for | | | | | | | |
| 12 | 103021_r_at | Cluster Incl AI317205: ui35g09 y1 Mus musculus cDNA 5 end/clone = IMAGE-1853344/clone_end = 5/gb = AI317205/ | P | 507.7 | P | 422.8 | P | 307.6 | P |
| | | gi = 4032472/ug = Mm 15918/len = 235/STRA = for | | | | | | | |
| 12 | 103056_at | Cluster Incl AI849721 UI-M-AL1-ahn-b-07-0-UI s1 Mus musculus cDNA, 3 end/clone = UI-M-AL1-ahn-b-07-0-UI/ | P | 410.4 | P | 491.3 | P | 207.9 | P |
| | | clone_end = 3/gb = AI849721/gi = 5493627/ug = Mm 1654/len = 352/STRA = for | | | | | | | |
| 12 | 103064_at | Cluster Incl AF016583: Mus musculus checkpoint kinase Chk1 (Chk1) mRNA, complete cds/cds = (22,1452)/gb = AF016583/ | P | 220.1 | P | 233.5 | P | 122.2 | P |
| | | gi = 2367670/ug = Mm 16753/len = 1962/STRA = for | | | | | | | |
| 12 | 103074_f_at | Cluster Incl AI842969 UI-M-AO1-aem-g-05-0-UI s1 Mus musculus cDNA, 3 end/clone = UI-M-AO1-aem-g-05-0-UI/ | P | 473.3 | P | 419.3 | P | 329.7 | P |
| | | clone_end = 3/gb = AI842969/gi = 5477182/ug = Mm 16976/len = 340/STRA = for | | | | | | | |
| 12 | 103086_at | Cluster Incl Y00208: Homeo box A5/cds = (74,886)/gb = Y00208/gi = 51371/ug = Mm 173/len = 1678/STRA = for | P | 280.9 | P | 167.1 | P | 208.9 | P |
| 12 | 103370_at | Cluster Incl AI848387 UI-M-AH1-ago-e-07-0-UI.s1 Mus musculus cDNA, 3 end/clone = UI-M-AH1-ago-e-07-0-UI/ | P | 1272.5 | P | 984.5 | P | 766.4 | P |
| | | clone_end = 3/gb = AI848367/gi = 5492268/ug = Mm 18555/len = 533/STRA = for | | | | | | | |
| 12 | 103400_at | Cluster Incl D50523 Mouse mRNA for TI-227/cds = UNKNOWN/gb = D50523/gi = 1166394/ug = Mm 18728/len = 3577/ | P | 777.6 | P | 669.4 | P | 514.5 | P |
| | | STRA = for | | | | | | | |
| 12 | 103654_at | Cluster Incl AB018374 Mus musculus GARP45 mRNA, complete cds/cds = (69,1289)/gb = AB018374/gi = 5263197/ | P | 694.1 | P | 447 | P | 217.7 | P |
| | | ug = Mm 20450/len = 1865/STRA = for | | | | | | | |
| 12 | 103657_i_at | Cluster Incl AI836552 UI-M-AP0-abe-e-08-0-UI.s1 Mus musculus cDNA, 3 end/clone = UI-M-AP0-abe-e-08-0-UI/ | P | 254.3 | P | 273 | P | 160.3 | P |
| | | clone_end = 3/gb = AI836552/gi = 5470765/ug = Mm.20559/len = 417/STRA = for | | | | | | | |
| 12 | 103874_r_at | Cluster Incl AA684456 vm64c06 s1 Mus musculus cDNA, 5 end/clone = IMAGE-1003018/clone_end = 5/gb = AA684456/ | P | 110.9 | P | 63.5 | P | 71.3 | P |
| | | gi = 2671042/ug = Mm.21173/len = 217/STRA = for | | | | | | | |
| 12 | 103892_r_at | Cluster Incl AI197161 ue51h10 r1 Mus musculus cDNA, 5 end/clone = IMAGE-1494691/clone_end = 5/gb = AI197161/ | P | 367.8 | P | 233.8 | P | 182.2 | P |
| | | gi = 3749767/ug = Mm 21288/len = 475/STRA = for | | | | | | | |
| 12 | 104046_at | Cluster Incl AI854141 UI-M-BH0-ajw-f-12-0-UI s1 Mus musculus cDNA, 3 end/clone = UI-M-BH0-ajw-f-12-0-UI/ | P | 674.5 | P | 640.1 | P | 628.3 | P |
| | | clone_end = 3/gb = AI854141/gi = 5498047/ug = Mm.21489/len = 577/STRA = for | | | | | | | |
| 12 | 104080_at | Cluster Incl AW212479 uo89c10 x1 Mus musculus cDNA, 3 end/clone = IMAGE-2649714/clone_end = 3/gb = AW212479/ | P | 922 | P | 898.5 | P | 594.5 | P |
| | | gi = 6518566/ug = Mm 21753/len = 163/STRA = for | | | | | | | |
| 12 | 104115_at | Cluster Incl AA867340: vx97a11 r1 Mus musculus cDNA, 5 end/clone = IMAGE-1293884/clone_end = 5/gb = AA867340/ | P | 589 | P | 581.5 | P | 397.3 | P |
| | | gi = 2962785/ug = Mm.21963/len = 308/STRA = for | | | | | | | |
| 12 | 104303_i_at | Cluster Incl AW124151 UI-M-BH2 1-apr-f-08-0-UI s1 Mus musculus cDNA, 3 end/clone = UI-M-BH2.1-apr-f-08-0-UI/ | P | 222.6 | P | 207.6 | P | 88 | P |
| | | clone_end = 3/gb = AW124151/gi = 6099681/ug = Mm.22358/len = 298/STRA = for | | | | | | | |
| 12 | 104304_r_at | Cluster Incl AW124151 UI-M-BH2 1-apr-f-08-0-UI s1 Mus musculus cDNA, 3 end/clone = UI-M-BH2.1-apr-f-08-0-UI/ | P | 207.5 | P | 197.9 | P | 158.7 | P |
| | | clone_end = 3/gb = AW124151/gi = 6099681/ug = Mm.22358/len = 298/STRA = for | | | | | | | |
| 12 | 104531_at | Cluster Incl X60304 Protein kinase C, delta/cds = (13,2037)/gb = X60304/gi = 53436/ug = Mm 2314/len = 2538/STRA = rev | P | 1149.2 | P | 979 | P | 518.8 | P |
| 12 | 104567_at | Cluster Incl AI848526 UI-M-AP1-agf-e-11-0-UI s2 Mus musculus cDNA, 3 end/clone = UI-M-AP1-agf-e-11-0-UI/ | P | 129.2 | P | 121.7 | P | 118.8 | P |
| | | clone_end = 3/gb = AI848526/gi = 5492432/ug = Mm 23880/len = 423/STRA = for | | | | | | | |
| 12 | 104578_f_at | Cluster Incl AI195392 ue72102 r1 Mus musculus cDNA, 5 end/clone = IMAGE-1496667/clone_end = 5/gb = AI195392/ | P | 1243.7 | P | 1345.5 | P | 785.7 | P |
| | | gi = 3747998/ug = Mm 23961/len = 262/STRA = for | | | | | | | |
| 12 | 104595_at | Cluster Incl AI846890 UI-M-AK1-aey-a-05-0-UI s1 Mus musculus cDNA, 3 end/clone = UI-M-AK1-aey-a-05-0-UI/ | P | 469.4 | P | 288.8 | P | 241 | P |
| | | clone_end = 3/gb = AI846890/gi = 5490796/ug = Mm.24025/len = 346/STRA = rev | | | | | | | |
| 12 | 104738_at | Cluster Incl D63784 Mouse mRNA for MIDA1, complete cds/cds = (112,1977)/gb = D63784/gi = 1060924/ug = Mm 2467/ | P | 478.4 | P | 378.8 | P | 294.4 | P |
| | | len = 2037/STRA = for | | | | | | | |
| 12 | 104745_at | Cluster Incl AA763874 vv48a12.r1 Mus musculus cDNA, 5 end/clone = IMAGE-1225630/clone_end = 5/gb = AA763874/ | P | 421.5 | P | 324.4 | P | 108.9 | P |
| | | gi = 2811396/ug = Mm.24719/len = 443/STRA = for | | | | | | | |
| 12 | 104749_at | Cluster Incl AW123623 UI-M-BH2.1-api-e-02-0-UI s1 Mus musculus cDNA, 3 end/clone = UI-M-BH2.1-api-e-02-0-UI/ | P | 208.3 | P | 216.5 | P | 164 | P |
| | | clone_end = 3/gb = AW123623/gi = 6099153/ug = Mm 24765/len = 224/STRA = for | | | | | | | |
| 12 | 94343_at | Cluster Incl AI604013 vr15c03 y1 Mus musculus cDNA, 5 end/clone = IMAGE-1120708/clone_end = 5/gb = AI604013/ | P | 286.3 | P | 230.8 | P | 195.5 | P |
| | | gi = 4613175/ug = Mm.25122/len = 164/STRA = for | | | | | | | |
| 12 | 94361_at | Cluster Incl AI836083 UI-M-AJ0-abb-f-06-0-UI s1 Mus musculus cDNA, 3 end/clone = UI-M-AJ0-abb-f-06-0-UI/ | P | 613.7 | P | 507 | P | 620.1 | P |
| | | clone_end = 3/gb = AI836083/gi = 5470296/ug = Mm.25264/len = 529/STRA = for | | | | | | | |
| 12 | 94985_at | Cluster Incl AI846392 UI-M-AN1-afa-h-02-0-UI s1 Mus musculus cDNA, 3 end/clone = UI-M-AN1-afa-h-02-0-UI/ | P | 547 | P | 452.8 | P | 344.9 | P |
| | | clone_end = 3/gb = AI846392/gi = 5490298/ug = Mm.27303/len = 399/STRA = for | | | | | | | |
| 12 | 95522_i_at | Cluster Incl AB024005.Mus musculus mRNA for KRAB-containing zinc-finger protein KRAZ2, complete cds/ | P | 420.4 | P | 322.3 | P | 242.2 | P |
| | | cds = (307,2127)/gb = AB024005/gi = 4514560/ug = Mm.27575/len = 2247/STRA = for | | | | | | | |
| 12 | 95530_at | Cluster Incl AW060250 UI-M-BH1-amv-e-11-0-UI s1 Mus musculus cDNA, 3 end/clone = UI-M-BH1-amv-e-11-0-UI/ | P | 324.9 | P | 234.8 | P | 237 | P |
| | | clone_end = 3/gb = AW060250/gi = 6008041/ug = Mm.27629/len = 581/STRA = for | | | | | | | |
| 12 | 95594_at | Cluster Incl AI847486 UI-M-AI1-afs-g-07-0-UI.s1 Mus musculus cDNA, 3 end/clone = UI-M-AI1-afs-g-07-0-UI/clone_end = 3/ | P | 468.6 | P | 460 | P | 382.2 | P |
| | | gb = AI847486/gi = 5491392/ug = Mm 27914/len = 349/STRA = for | | | | | | | |
| 12 | 95611_at | Cluster Incl AA726364: vu90e02 r1 Mus musculus cDNA, 5 end/clone = IMAGE-1209434/clone_end = 5/gb = AA726364/ | P | 2017.1 | P | 1783 | P | 1621.3 | P |
| | | gi = 2744071/ug = Mm.27994/len = 374/STRA = for | | | | | | | |
| 12 | 95613_at | Cluster Incl AW122573 UI-M-BH2 2-aox-f-07-0-UI s1 Mus musculus cDNA, 3 end/clone = UI-M-BH2 2-aox-f-07-0-UI/ | P | 572.8 | P | 501.6 | P | 394.1 | P |
| | | clone_end = 3/gb = AW122573/gi = 6098098/ug = Mm.28001/len = 448/STRA = rev | | | | | | | |
| 12 | 96135_at | Cluster Incl AA833425: ub58d03 r1 Mus musculus cDNA, 5 end/clone = IMAGE-1381925/clone_end = 5/gb = AA833425/ | P | 1656.3 | P | 1236.3 | P | 800.7 | P |
| | | gi = 2907153/ug = Mm.28149/len = 490/STRA = for | | | | | | | |
| 12 | 96152_at | Cluster Incl AI645561: vt98h08 x1 Mus musculus cDNA, 3 end/clone = IMAGE-1179231/clone_end = 3/gb = AI645561/ | P | 630.9 | P | 576.4 | P | 293.1 | P |
| | | gi = 4724036/ug = Mm 28256/len = 332/STRA = for | | | | | | | |
| 12 | 96755_at | Cluster Incl AA638002: vr30d10 r1 Mus musculus cDNA, 5 end/clone = IMAGE-1122163/clone_end = 5/gb = AA638002/ | P | 875.1 | P | 802.5 | P | 648.4 | P |
| | | gi = 2561590/ug = Mm 28982/len = 136/STRA = for | | | | | | | |
| 12 | 96773_at | Cluster Incl AW125408: UI-M-BH2 3-aqh-d-12-0-UI s1 Mus musculus cDNA, 3 end/clone = UI-M-BH2 3-aqh-d-12-0-UI/ | P | 332.4 | P | 285.7 | P | 308.8 | P |
| | | clone_end = 3/gb = AW125408/gi = 6100938/ug = Mm 29036/len = 480/STRA = for | | | | | | | |
| 12 | 96836_r_at | Cluster Incl AI447619: mr29c12.x1 Mus musculus cDNA, 3 end/clone = IMAGE-598870/clone_end = 3/gb = AI447619/ | P | 58.3 | P | 56.2 | P | 30.7 | P |
| | | gi = 4305257/ug = Mm.29434/len = 217/STRA = for | | | | | | | |
| 12 | 97394_at | Cluster Incl AA794509: vr46h05 s1 Mus musculus cDNA, 5 end/clone = IMAGE-1123737/clone_end = 5/gb = AA794509/ | P | 335.3 | P | 261 | P | 203 | P |
| | | gi = 2857464/ug= Mm 29825/len = 571/STRA = for | | | | | | | |
| 12 | 97947_at | Cluster Incl AI836959: UI-M-AJ0-aaz-b-08-0-UI s1 Mus musculus cDNA, 3 end/clone = UI-M-AJ0-aaz-b-08-0-UI/ | P | 613.2 | P | 437.5 | P | 355 | P |
| | | clone_end = 3/gb = AI836959/gi = 5471172/ug = Mm 3037/len = 449/STRA = for | | | | | | | |
| 12 | 97991_at | Cluster Incl X02452: Mouse Ki-ras cellular oncogene exon 1 from Y1 adrenal tumor cells (and joined CDS)/cds = (11,577)/ | P | 418.2 | P | 231.4 | P | 150.1 | P |
| | | gb = X02452/gi = 52798/ug= Mm 31530/len = 1722/STRA = for | | | | | | | |
| 12 | 98013_at | Cluster Incl AA666464: vm48a05.r1 Mus musculus cDNA, 5 end/clone = IMAGE-1001456/clone_end = 5/gb = AA666464/ | P | 267.9 | P | 168.9 | P | 231.8 | P |
| | | gi = 2625165/ug = Mm 3224/len = 164/STRA = for | | | | | | | |
| 12 | 98438_f_at | Cluster Incl X16202 Mouse Q4 class 1 MHC gene (exon 5)/cds = (0,91)/gb = X16202/gi = 53862/ug = Mm 34421/len = 116/ | P | 1880.4 | P | 888.1 | P | 378.3 | P |
| | | STRA = for | | | | | | | |
| 12 | 98441_at | Cluster Incl L23971 Fragile X mental retardation syndrome 1 homolog/cds = (123,1967)/gb = L23971/gi = 398044/ | P | 468 | P | 353.6 | P | 209.1 | P |
| | | ug = Mm.3451/len = 4240/STRA = rev | | | | | | | |
| 12 | 98491_at | Cluster Incl AI286904: ui78d09 y1 Mus musculus cDNA, 5 end/clone = IMAGE-1888529/clone_end = 5/gb = AI286904/ | P | 289.8 | P | 301.8 | P | 233.5 | P |
| | | gi = 3926657/ug_Mm 35596/len = 352/STRA = for | | | | | | | |
| 12 | 98509_at | Cluster Incl AI834979 UI-M-AM1-afw-e-03-0-UI s1 Mus musculus cDNA, 3 end/clone = UI-M-AM1-afw-e-03-0-UI/ | P | 484 | P | 357.8 | P | 352.1 | P |
| | | clone_end = 3/gb = AI834979/gi = 5469192/ug = Mm.3584/len = 451/STRA = for | | | | | | | |
| 12 | 99462_at | Cluster Incl D38046 Mouse mRNA for typeII DNA topoisomerase beta isoform, complete cds/cds = (46,4884)/gb = D38046/ | P | 446.5 | P | 473.4 | P | 345.6 | P |
| | | gi = 533330/ug = Mm.4093/len = 5207/STRA = for | | | | | | | |
| 12 | 99467_at | Cluster Incl AI846922.UI-M-AK1-aey-d-05-0-UI s1 Mus musculus cDNA, 3 end/clone = UI-M-AK1-aey-d-05-0-UI/ | P | 569.5 | P | 363.3 | P | 335.7 | P |
| | | clone_end = 3/gb = AI846922/gi = 5490828/ug = Mm 41224/len = 381/STRA = for | | | | | | | |
| 12 | 99935_at | Cluster Incl D14340 Tight junction protein 1/cds = 403,5640)/gb = D14340/gi = 303709/ug = Mm 4342/len = 7046/STRA = for | P | 604 | P | 442.3 | P | 348.7 | P |
| 12 | 99949_at | Cluster Incl AW060324 UI-M-BH1-anh-a-06-0-UI s1 Mus musculus cDNA, 3 end/clone = UI-M-BH1-anh-a-06-0-UI/ | P | 188.1 | P | 188.4 | P | 127.9 | P |
| | | clone_end = 3/gb = AW060324/gi = 6008075/ug = Mm 43681/len = 475/STRA = for | | | | | | | |
| 12 | 99959_at | Cluster Incl AW061337 UI-M-BH1-anw-f-11-0-UI.s1 Mus musculus cDNA, 3 end/clone = UI-M-BH1-anw-f-11-0-UI/ | P | 150.8 | P | 168.8 | P | 112.4 | P |
| | | clone_end = 3/gb = AW061337/gi = 6009088/ug = Mm 4410/len = 475/STRA = for | | | | | | | |
| 12 | 99985_at | Cluster Incl AB027565 Mus musculus TXNRD1 mRNA for thioredoxin reductase 1, complete cds/cds = (158,1657)/ | P | 634.6 | P | 517.1 | P | 510.7 | P |
| | | gb = AB027565/gi = 6467192/ug= Mm 44552/len = 3328/STRA = for | | | | | | | |
| 12 | 99990_at | Cluster Incl U28789 Mus musculus p53-associated cellular protein PACT mRNA, partial cds/cds = (0,4764)/gb = U28789/ | P | 893.6 | P | 809 | P | 403.7 | P |
| | | gi = 1546778/ug = Mm 4480/len = 5191/STRA = for | | | | | | | |
| 12 | 100400_at | Cluster Incl AW124329.UI-M-BH2 1-apq-e-12-0-UI s1 Mus musculus cDNA, 3 end/clone = UI-M-BH2 1-apq-e-12-0-UI/ | P | 206.3 | P | 170.8 | P | 113.7 | P |
| | | clone_end = 3/gb = AW124329/gi = 6099859/ug = Mm 464/len = 236/STRA = for | | | | | | | |
| 12 | 100464_at | Cluster Incl AI840585: UI-M-AM0-adq-a-06-0-UI s1 Mus musculus cDNA, 3 end/clone = UI-M-AM0-adq-a-06-0-UI/ | P | 460.8 | P | 276.6 | P | 208.2 | P |
| | | clone_end = 3/gb = AI840585/gi = 5474798/ug = Mm 491/len = 447/STRA = for | | | | | | | |
| 12 | 100469_at | Cluster Incl D78642 Nuclear transcription factor-Y alpha/cds = UNKNOWN/gb = D78642/gi = 1304151/ug = Mm 4929/ | P | 263.4 | P | 266.9 | P | 237.8 | P |
| | | len = 2121/STRA = for | | | | | | | |
| 12 | 100954_at | Cluster Incl AF057287 Mus musculus RAB/Rip protein mRNA, partial cds/cds = (0,983)/gb = AF057287/gi = 3063650/ | P | 245.1 | P | 237.7 | P | 147.8 | P |
| | | ug = Mm 6461/len = 1598/STRA = for | | | | | | | |
| 12 | 100980_at | Cluster Incl U58512 Mus musculus Rho-associated, coiled-coil forming protein kinase p160 ROCK-1 mRNA, complete cds/ | P | 355.6 | P | 163.3 | P | 114.2 | P |
| | | cds = (0,4064)/gb = U58512/gi = 1514695/ug = Mm 6710/len = 4065/STRA = for | | | | | | | |
| 12 | 101459_at | Cluster Incl L10410 Chromodomain helicase DNA binding protein 1/cds = (170,5305)/gb = L10410/gi = 455014/ug = Mm 8137/ | P | 409.5 | P | 304.6 | P | 266 | P |
| | | len = 5349/STRA = for | | | | | | | |
| 12 | 101890_f_at | Cluster Incl U53208 Zuotin related factor 1/cds = (68,1612)/gb = U53208/gi = 1256831/ug = Mm 88678/len = 1995/STRA = for | P | 398.2 | P | 364.6 | P | 381.2 | P |
| 12 | 101894_s_at | Cluster Incl X71978 Fused toes/cds = (213,1091)/gb = X71978/gi = 311631/ug = Mm.88777/len = 1793/STRA = for | P | 279.9 | P | 193.7 | P | 230.2 | P |
| 12 | 102645_at | Cluster Incl AI155285.ud84b11 r1 Mus musculus cDNA, 5 end/clone = IMAGE-1477533/clone_end = 5/gb = AI155285/ | P | 145.5 | P | 121.6 | P | 126 | P |
| | | gi = 3683754/ug = Mm 13146/len = 325/STRA = for | | | | | | | |
| 12 | 102647 _g_at | Cluster Incl AW122284 UI-M-BH2 2-aos-b-02-0-UI s1 Mus musculus cDNA, 3 end/clone = UI-M-BH2 2-aos-b-02-0-UI/ | P | 782.5 | P | 573.4 | P | 607.5 | P |
| | | clone_end = 3/gb = AW122284/gi = 8097765/ug = Mm 13148/len = 336/STRA = for | | | | | | | |
| 12 | 103300_at | Cluster Incl AW124239 UI-M-BH2 1-aph-e-12-0-UI.s1 Mus musculus cDNA, 3 end/clone = UI-M-BH2.1-aph-e-12-0-UI/ | P | 572.3 | P | 432.2 | P | 232.8 | P |
| | | clone_end = 3/gb = AW124239/gi = 6099769/ug = Mm 21081/len = 279/STRA = for | | | | | | | |
| 12 | 103309_at | Cluster Incl AI853977 UI-M-BH0-aiv-f-01-0-UI s1 Mus musculus cDNA, 3 end/clone = UI-M-BH0-aiv-f-01-0-UI/ | P | 382.4 | P | 354.4 | P | 260.5 | P |
| | | clone_end = 3/gb = AI853977/gi = 5497883/ug = Mm 21158/len = 265/STRA = for | | | | | | | |
| 12 | 103459_at | Cluster Incl AW124544 UI-M-BH2 1-apo-h-11-0-UI s1 Mus musculus cDNA, 3 end/clone = UI-M-BH2.1-apo-h-11-0-UI/ | P | 549.6 | P | 635 | P | 445.3 | P |
| | | clone end = 3/gb = AW124544/gi = 6100074/ug = Mm.21688/len = 256/STRA = for | | | | | | | |
| 12 | 103500_at | Cluster Incl AI553463: vw56g01 x1 Mus musculus cDNA, 3 end/clone = IMAGE-1247856/clone_end = 3/gb = AI553463/ | P | 316.4 | P | 324.4 | P | 280.3 | P |
| | | gi = 4485826/ug = Mm 22344/len = 457/STRA = rev | | | | | | | |
| 12 | 103525_at | Cluster Incl AA981725: ua28h11 r1 Mus musculus cDNA, 5 end/clone = IMAGE-1348101/clone_end = 5/gb = AA981725/ | P | 421.3 | P | 294.1 | P | 264.3 | P |
| | | gi = 3160494/ug = Mm 2290/len = 433/STRA = for | | | | | | | |
| 12 | 104163_at | Cluster Incl AA711002: vt52g08.r1 Mus musculus cDNA, 5 end/clone = IMAGE-1166750/clone_end = 5/gb = AA711002/ | P | 467 | P | 403.8 | P | 362.9 | P |
| | | gi = 2720920/ug = Mm 27130/len = 377/STRA = for | | | | | | | |
| 12 | 104179_at | Cluster Incl AI788669 uk47h11 x1 Mus musculus cDNA, 3 end/clone = IMAGE-1972197/clone_end = 3/gb = AI788669/ | P | 304.2 | P | 346.8 | P | 162.9 | P |
| | | gi = 5336385/ug = Mm 27308/len = 503/STRA = for | | | | | | | |
| 12 | 104212_at | Cluster Incl AA607767 vo08g02 r1 Mus musculus cDNA, 5 end/clone = IMAGE-1040882/clone_end = 5/gb = AA607767/ | P | 319.2 | P | 240.2 | P | 204 | P |
| | | gi = 2455202/ug = Mm 27763/len = 509/STRA = for | | | | | | | |
| 12 | 104399_at | Cluster Incl AI850965 UI-M-BH0-akd-f-12-0-UI s1 Mus musculus cDNA, 3 end/clone = UI-M-BH0-akd-f-12-0-UI/ | P | 416.7 | P | 270.4 | P | 258.5 | P |
| | | clone_end = 3/gb = AI850965/gi = 5494871/ug = Mm 2866/len = 323/STRA = for | | | | | | | |
| 12 | 104404_at | Cluster Incl AA693221 vr58c04 s1 Mus musculus cDNA, 5 end/clone = IMAGE-1124838/clone_end = 5/gb = AA693221/ | P | 237.6 | P | 170.5 | P | 154.6 | P |
| | | gi = 2694159/ug = Mm 28701/len = 453/STRA = for | | | | | | | |
| 12 | 92243_at | Cluster Incl AA117417: mp90d10.r1 Mus musculus cDNA, 5 end/clone = IMAGE-576499/clone_end = 5/gb = AA117417/ | P | 591.3 | P | 566.1 | P | 335.8 | P |
| | | gi = 1672496/ug = Mm.34910/len = 494/STRA = for | | | | | | | |
| 12 | 92282_at | Cluster lncl AI646301: vs77f11.x1 Mus musculus cDNA, 3 end/clone = IMAGE-1152333/clone_end = 3/gb = AI646301/ | P | 245.6 | P | 223.2 | P | 212.5 | P |
| | | gi = 4724776/ug = Mm 3702/len = 305/STRA = for | | | | | | | |
| 12 | 92360_at | Cluster Incl U43900.Signal transducing adaptor molecule (SH3 domain and ITAM motif) 1/cds = (42,1688)/gb = U43900/ | P | 594.9 | P | 544.9 | P | 448.9 | P |
| | | gi = 1556460/ug = Mm 3965/len = 2747/STRA = for | | | | | | | |
| 12 | 92480_f_at | Cluster Incl AB024004.Mus musculus mRNA for KRAB-containing zinc-finger protein KRAZ1, complete cds/ | P | 223.8 | P | 161.6 | P | 131.1 | P |
| | | cds = (116,2074)/gb = AB024004/gi = 4514558/ug = Mm.42140/len = 2191/STRA = for | | | | | | | |
| 12 | 92487_at | Cluster Incl AB023419 Mus musculus mRNA for mSox7, complete cds/cds = (50,1192)/gb = AB023419/gi = 5103026/ | P | 691.4 | P | 618.7 | P | 601.1 | P |
| | | ug = Mm 42162/len = 3266/STRA = for | | | | | | | |
| 12 | 93426_at | Cluster Incl AA199023 mU13a03 r1 Mus musculus cDNA, 5 end/clone = IMAGE-639244/clone_end = 5/gb = AA199023/ | P | 614.3 | P | 564.3 | P | 522.4 | P |
| | | gi = 1794518/ug = Mm 6488/len = 203/STRA = for | | | | | | | |
| 12 | 93455_s_at | Cluster Incl X56848.M musculus mRNA for bone morphogenetic protein 4 (BMP-4)/cds = (356,1582)/gb = X56848/gi = 50180/ | P | 435 | P | 608.5 | P | 352.3 | P |
| | | ug = Mm.6813/len = 1785/STRA = for | | | | | | | |
| 12 | 93471_at | Cluster Incl AI594427-vo35a08 y1 Mus musculus cDNA, 5 end/clone = IMAGE-1051882/clone_end = 5/gb = AI594427/ | P | 440.4 | P | 218.9 | P | 214.4 | P |
| | | gI = 4603475/ug = Mm 7033/len = 447/STRA = for | | | | | | | |
| 12 | 93701_at | Cluster Incl AA982124.vx56f01.r1 Mus musculus cDNA, 5 end/clone = IMAGE-1279225/clone_end = 5/gb = AA982124/ | P | 291.8 | P | 259.3 | P | 244.7 | P |
| | | gi = 3160243/ug = Mm.86261/len = 474/STRA = for | | | | | | | |
| 12 | 93868_at | Cluster Incl AL021127 Mus musculus cosmid CMX137 containing part of Nsdhl/cds = (0,578)/gb = AL021127/gi = 2769543/ | P | 211.7 | P | 206.8 | P | 156.6 | P |
| | | ug = Mm 87851/len = 579/STRA = for | | | | | | | |
| 12 | 94110_f_at | Cluster Incl D10627.Mus musculus mRNA, for zinc finger protein, partial cds/cds = (0,1040)/gb = D10627/gi = 220636/ | P | 412.6 | P | 343.3 | P | 324.1 | P |
| | | ug = Mm 10509/len = 1041/STRA = for | | | | | | | |
| 12 | 95282_at | Cluster Incl J04633 Mouse heat shock protein 86 mRNA, complete cds, and 28S ribosomal RNA, partial sequence/ | P | 2042.3 | P | 2236.2 | P | 1436.6 | P |
| | | cds = (260,2461)/gb = J04633/gi = 194030/ug = Mm 1843/len = 2975/STRA = for | | | | | | | |
| 12 | 100760_at | Cluster lncl X97986 M musculus mRNA for desmocollin type 1/cds = (151,2649)/gb = X97986/gi = 1707586/ug = Mm 909/ | P | 723.3 | P | 496.3 | P | 737.9 | P |
| | | len = 2987/STRA = for | | | | | | | |
| 12 | 101634_at | Cluster Incl M33212 Mouse nucleolar protein N038 mRNA, complete cds/cds = (78,956)/gb = M33212/gi = 200010/ | P | 3090.2 | P | 3288 | P | 2901.3 | P |
| | | ug = Mm 6343/len = 1260/STRA = for | | | | | | | |
| 12 | 92592_at | Cluster Incl M25558 Glycerolphosphate dehydrogenase 1, cytoplasmic adult/cds = (0,1049)/gb = M25558/gi = 193601/ | P | 870.8 | P | 568.9 | P | 468.7 | P |
| | | ug = Mm 10669/len = 1050/STRA = for | | | | | | | |
| 12 | 92871_at | Cluster Incl AW121840: UI-M-BH2 3-aoi-a-03-0-UI s1 Mus musculus cDNA, 3 end/clone = UI-M-BH2 3-aoi-a-03-0-UI/ | P | 323.9 | P | 236.4 | P | 222.8 | P |
| | | clone_end = 3/gb = AW121840/gi = 6097214/ug = Mm 14352/len = 466/STRA = for | | | | | | | |
| 12 | 95416_at | Cluster Incl AI642184.uc46h07.x1 Mus musculus cDNA, 3 end/clone = IMAGE-1401085/clone_end = 3/gb = AI642184/ | P | 328 | P | 277.4 | P | 200.5 | P |
| | | gi = 4720659/ug = Mm.24280/len = 642/STRA = for | | | | | | | |
| 12 | 97273_at | Cluster Incl AI845953: UI-M-AK1-aex-h-04-0-UI s1 Mus musculus cDNA, 3 end/clone = UI-M-AK1-aex-h-04-0-UI/ | P | 740 | P | 635.9 | P | 218.1 | A |
| | | clone_end = 3/gb = AI845953/gi = 5489859/ug = Mm 27932/len = 357/STRA = for | | | | | | | |
| 12 | 96861_at | Cluster Incl AI852376.UI-M-BH0-ajo-e-04-0-UI.s1 Mus musculus cDNA, 3 end/clone = UI-M-BH0-ajo-e-04-0-UI/ | P | 239.3 | P | 252.9 | P | 137.5 | P |
| | | clone_end = 3/gb = AI852376/gi = 5496282/ug = Mm 30052/len = 348/STRA = for | | | | | | | |
| 12 | 97506_at | Cluster Incl AI043016 uc87a04.x1 Mus musculus cDNA, 3 end/clone = IMAGE-1432590/clone_end = 3/gb = AI043016/ | A | 286.6 | P | 244.1 | P | 205.7 | P |
| | | gi = 3286276/ug = Mm 31512/len = 532/STRA = for | | | | | | | |
| 12 | 98135_r_at | Cluster Incl AI851008.UI-M-BH0-ajv-c-01-0-UI.s1 Mus musculus cDNA, 3 end/clone = UI-M-BH0-ajv-c-01-0-UI/ | P | 52.2 | P | 48 | P | 47.9 | P |
| | | clone_end = 3/gb = AI851008/gi = 5494914/ug = Mm.35492/len = 438/STRA = for | | | | | | | |
| 12 | 99662_at | Cluster Incl AI194767 ui56d10.x1 Mus musculus cDNA, 3 end/clone = IMAGE-1886419/clone_end = 3/gb = AI194767/ | P | 216.8 | P | 125.4 | P | 84.9 | A |
| | | gi = 3747373/ug = Mm 4379/len = 525/STRA = for | | | | | | | |
| 12 | 101020_at | Cluster Incl AI842667 UI-M-AO1-aeo-a-04-0-UI.s1 Mus musculus cDNA, 3 end/clone = UI-M-AO1-aeo-a-04-0-UI/ | P | 258.8 | P | 142.4 | P | 90.6 | A |
| | | clone_end = 3/gb = AI842667/gi = 5476880/ug = Mm.684/len = 284/STRA = for | | | | | | | |
| 12 | 101079_at | Cluster Incl AF093140 Mus musculus tip associating protein (Tap) mRNA, complete cds/cds = (105,1961)/gb = AF093140/ | P | 225.1 | P | 191.2 | P | 139.3 | A |
| | | gi = 3719434/ug = Mm 7271/len = 2034/STRA = for | | | | | | | |
| 12 | 103063_at | Cluster Incl Z67747 M. musculus mRNA for ZT3 zinc finger factor/cds = (0,1570)/gb = Z67747/gi = 1061193/ug = Mm 16650/ | P | 158.8 | P | 132.1 | P | 97.2 | P |
| | | len = 2547/STRA = for | | | | | | | |
| 12 | 103598_at | Cluster Incl U91922: D-E-A-D (aspartate-glutamate-alanine-aspartate) box polypeptide 9/cds = (107,4249)/gb = U91922/ | P | 358.8 | P | 182.3 | P | 144.5 | A |
| | | gi = 2961455/ug = Mm 20000/len = 4498/STRA = for | | | | | | | |
| 12 | 103665_at | Cluster Incl AW122523 UI-M-BH2.2-aox-b-05-0-UI s1 Mus musculus cDNA, 3 end/clone = UI-M-BH2 2-aox-b-05-0-UI/ | P | 480.1 | P | 433.6 | P | 285.7 | P |
| | | clone_end = 3/gb = AW122523/gi = 6098047/ug = Mm 20697/len = 423/STRA = for | | | | | | | |
| 12 | 104298_at | Cluster Incl AI842544 UI-M-AQ1-aea-e-05-0-UI.s1 Mus musculus cDNA, 3 end/clone = UI-M-AQ1-aea-e-05-0-UI/ | P | 164.6 | P | 116.1 | P | 131.5 | P |
| | | clone end = 3/gb = AI842544/gi = 5476757/ug = Mm 22337/len = 457/STRA = for | | | | | | | |
| 12 | 104536_at | Cluster Incl U60530 MAD homolog 2, (Drosophila)/cds = (59,1462)/gb = U60530/gi = 1407781/ug = Mm 2334/len = 1639/ | P | 398.2 | P | 293.2 | P | 141.2 | A |
| | | STRA = for | | | | | | | |
| 12 | 95521_s_at | Cluster Incl AB024005 Mus musculus mRNA for KRAB-containing zinc-finger protein KRAZ2, complete cds/ | P | 203.9 | P | 197.5 | P | 127.1 | P |
| | | cds = (307,2127)/gb = AB024005/gi = 4514560/ug = Mm 27575/len = 2247/STRA = for | | | | | | | |
| 12 | 100459_at | Cluster Incl U66887: RAD50 homolog (S cerevisiae)/cds = (242,4180)/gb = U66887/gi = 1575574/ug = Mm 4888/len = 5088/ | P | 246.4 | P | 205.8 | P | 223 | P |
| | | STRA = for | | | | | | | |
| 12 | 102872_f_at | Cluster Incl X74855 Zinc finger protein 51/cds = (350,2482)/gb = X74855/gi = 488832/ug = Mm 14812/len = 2640/STRA = for | P | 168 | P | 104.1 | P | 80.2 | P |
| 12 | 102887_at | Cluster Incl U94331 Mus musculus osteoprotegerin (OPG) mRNA, complete cds/cds = (90,1295)/gb = U94331/gi = 2072182/ | P | 334.2 | P | 331 | P | 206.3 | P |
| | | ug = Mm 15383/len = 1325/STRA = for | | | | | | | |
| 12 | 103745_at | Cluster Incl AW227027.um65d05 y1 Mus musculus cDNA, 5 end/clone = IMAGE-2286249/clone_end = 5/gb = AW227027/ | P | 206 | P | 189 | P | 184.2 | P |
| | | gi = 6556323/ug = Mm.24475/len = 457/STRA = for | | | | | | | |
| 12 | 104419_at | Cluster Incl AI132380 ue24a03.x1 Mus musculus cDNA, 3 end/clone = IMAGE-1481260/clone_end = 3/gb = AI132380/ | P | 235.3 | P | 174.1 | P | 142.6 | P |
| | | gi = 3602396/ug = Mm.28928/len = 441/STRA = for | | | | | | | |
| 12 | 104501_at | Cluster Incl AI849048 UI-M-AH1-agw-b-07-0-UI.s1 Mus musculus cDNA, 3 end/clone = UI-M-AH1-agw-b-07-0-UI/ | P | 101.2 | P | 121.8 | P | 59.1 | P |
| | | clone_end = 3/gb = AI849048/gi = 5492954/ug = Mm 3055/len = 421/STRA = rev | | | | | | | |
| 12 | 92408_at | Cluster Incl AI891514: ul59e08.x1 Mus musculus cDNA, 3 end/clone = IMAGE-2123366/clone_end = 3/gb = AI891514/ | P | 261.9 | P | 218 | P | 140.3 | A |
| | | gi = 5597416/ug = Mm.41046/len = 479/STRA = for | | | | | | | |
| 12 | 92649_at | Cluster Incl Y18505: Mus musculus mRNA for XAP-5 protein/cds = (147,1166)/gb = Y18505/gi = 5327064/ug = Mm.4370/ | P | 304.5 | P | 269.3 | P | 158.3 | A |
| | | len = 1348/STRA = for | | | | | | | |
| 12 | 95289_f_at | Cluster Incl AA189811: mt97g08 r1 Mus musculus cDNA, 5 end/clone = IMAGE-637886/clone_end = 5/gb = AA189811/ | P | 70.8 | P | 69.1 | P | 59.4 | P |
| | | gi = 1776416/ug = Mm.1882/len = 365/STRA = for | | | | | | | |
| 12 | 98761_i_at | Cluster Incl L20450 Mus musculus DNA-binding protein mRNA, complete cds/cds = (93,1733)/gb = L20450/gi = 437443/ | P | 269 | P | 175.8 | P | 148.4 | P |
| | | ug = Mm 4596/len = 2768/STRA = for | | | | | | | |
| 12 | 101294_g_at | Cluster Incl Z84471 M musculus G6pd-2 gene/cds = (0,1541)/gb = Z84471/gi = 1806125/ug = Mm 56918/len = 1542/ | P | 274.2 | P | 331.6 | P | 188.7 | P |
| | | STRA = for | | | | | | | |
| 12 | 93568_i_at | Cluster Incl AI853444 UI-M-BH0-ajy-d-09-0-UI s1 Mus musculus cDNA, 3 end/clone = UI-M-BH0-ajy-d-09-0-UI/ | P | 118.9 | P | 72.1 | P | 85.3 | P |
| | | clone_end = 3/gb = AI853444/gi = 5497350/ug = Mm 204/len = 363/STRA = for | | | | | | | |
| 12 | 94009_at | Cluster Incl AI848888 UI-M-AJ1-ahb-g-12-0-UI s1 Mus musculus cDNA, 3 end/clone = UI-M-AJ1-ahb-g-12-0-UI/ | P | 38.8 | P | 28.7 | A | 62.9 | A |
| | | clone_end = 3/gb = AI848888/gi = 5492794/ug = Mm.21811/len = 365/STRA = for | | | | | | | |
| 12 | 94917_at | Cluster Incl AI844932 UI-M-BG0-ahv-f-12-0-UI s1 Mus musculus cDNA, 3 end/clone = UI-M-BG0-ahv-f-12-0-UI/ | P | 120.7 | P | 111.8 | P | 92.3 | M |
| | | clone_end = 3/gb = AI844932/gi = 5488838/ug = Mm 24156/len = 407/STRA = for | | | | | | | |
| 12 | 95483_at | Cluster Incl AW123318 UI-M-BH2.1-apf-b-03-0-UI.s1 Mus musculus cDNA, 3 end/clone = UI-M-BH2 1-apf-b-03-0-UI/ | A | 464.6 | P | 341.8 | A | 286.5 | P |
| | | clone_end = 3/gb = AW123318/gi = 6098848/ug = Mm 24874/len = 371/STRA = for | | | | | | | |
| 12 | 96052_at | Cluster Incl AI852457 UI-M-BH0-aip-e-04-0-UI s1 Mus musculus cDNA, 3 end/clone = UI-M-BH0-aip-e-04-0-UI/ | A | 428.7 | P | 348.2 | P | 204.2 | A |
| | | clone_end = 3/gb = AI852457/gi = 5496363/ug = Mm 26166/len = 426/STRA = for | | | | | | | |
| 12 | 97243_at | Cluster Incl U74079 Mus musculus protein co-factor mRNA, complete cds/cds = (137,1204)/gb = U74079/gi = 1644403/ | P | 870 | P | 1066.1 | P | 619.7 | A |
| | | ug = Mm 27842/len = 1901/STRA = for | | | | | | | |
| 12 | 98959_at | Cluster Incl AW120623 UI-M-BH2.3-anx-h-09-0-UI s1 Mus musculus cDNA, 3 end/clone = UI-M-BH2 3-anx-h-09-0-UI/ | P | 350.3 | P | 290.4 | P | 217.2 | M |
| | | clone_end = 3/gb = AW120623/gi = 6095956/ug = Mm 28773/len = 308/STRA = for | | | | | | | |
| 12 | 94526_at | Cluster Incl AI848453 UI-M-AH1-ags-f-11-0-UI s1 Mus musculus cDNA, 3 end/clone = UI-M-AH1-ags-f-11-0-UI/ | P | 309.8 | P | 384.6 | P | 155.1 | A |
| | | clone_end = 3/gb = AI848453/gi = 5492359/ug = Mm.29032/len = 495/STRA = for | | | | | | | |
| 12 | 98539_at | Cluster Incl AF071312 Mus musculus COP9 complex subunit 2 (COPS2) mRNA, complete cds/cds = (12,1334)/ | P | 127.6 | P | 109.5 | P | 47.3 | A |
| | | gb = AF071312/gi = 3309165/ug = Mm 3596/len = 1929/STRA = for | | | | | | | |
| 12 | 103321_at | Cluster Incl AI536457 vu57h06 x1 Mus musculus cDNA, 3 end/clone = IMAGE-1195547/clone_end = 3/gb = AI536457/ | P | 177.3 | P | 30.6 | P | −0.4 | A |
| | | gi = 4450592/ug = Mm 17873/len = 435/STRA = for | | | | | | | |
| 12 | 104285_at | Cluster Incl M62766 3-hydroxy-3-methylglutaryl-coenzyme A reductase/cds = (0,674)/gb = M62766/gi = 193881/ | P | 192.6 | P | 226.4 | P | 143.6 | A |
| | | ug = Mm 2226/len = 1045/STRA = for | | | | | | | |
| 12 | 98966_at | Cluster Incl L42996 Mus musculus nuclear-encoded mitochondrial acyltransferase mRNA, complete cds/cds = (15,1463)/ | P | 135 | P | 119.1 | P | 79.9 | A |
| | | gb = L42996/gi = 1100214/ug = Mm 3636/len = 2664/STRA = for | | | | | | | |
| 12 | 100512_at | Cluster Incl AI838853: UI-M-AN0-ack-b-11-0-UI s1 Mus musculus cDNA, 3 end/clone = UI-M-AN0-ack-b-11-0-UI/ | P | 281.4 | P | 322 | P | 168.7 | A |
| | | clone_end = 3/gb = AI838853/gi = 5473066/ug = Mm 52067/len = 397/STRA = for | | | | | | | |
| 12 | 102197_at | Cluster Incl AJ222586 Mus musculus mRNA for NEFA protein, complete cds/cds = (287,1549)/gb = AJ222586/gi = 2612796/ | P | 125.9 | P | 108.2 | P | 4.1 | A |
| | | ug = Mm.9901/len = 1655/STRA = for | | | | | | | |
| 12 | 103260_at | Cluster Incl AI838553: UI-M-AL0-abp-d-10-0-UI s1 Mus musculus cDNA, 3 end/clone = UI-M-AL0-abp-d-10-0-UI/ | P | 81.5 | P | 89.8 | P | 36 | A |
| | | clone_end = 3/gb = AI838553/gi = 5472731/ug = Mm 208/len = 491/STRA = rev | | | | | | | |
| 12 | 95318_at | Cluster Incl AF045565 Zinc finger protein 105/cds = (231,1805)/gb = AF045565/gi = 3483132/ug = Mm 20430/len = 2002/ | P | 215.2 | P | 229.8 | P | 180.7 | M |
| | | STRA = for | | | | | | | |
| 12 | 96560_at | Cluster Incl AA648027 vn68h05 r1 Mus musculus cDNA, 5 end/clone = IMAGE-1026393/clone_end = 5/gb = AA648027/ | P | 584 | P | 409.4 | P | 341.8 | A |
| | | gi = 2574456/ug = Mm 28931/len = 419/STRA = for | | | | | | | |
| 12 | 99828_at | Cluster Incl AA673252 vp49C07 r1 Mus musculus cDNA, 3 end/clone = IMAGE-1080012/clone_end = 3/gb = AA673252/ | P | 215.2 | P | 201.7 | P | 146.8 | P |
| | | gi = 2646534/ug = Mm 6148/len = 370/STRA = rev | | | | | | | |
| 12 | 93502_r_at | Cluster Incl AF058955 Mus musculus ATP-specific succinyl-CoA synthetase beta subunit (Scs) mRNA, partial cds/ | P | 254.1 | P | 102.2 | P | 126.3 | A |
| | | cds = (0,1280)/gb = AF058955/gi = 3766200/ug = Mm 19154/len = 1991/STRA = for | | | | | | | |
| 12 | 95660_at | Cluster Incl AI851815: UI-M-BH0-ais-c-07-0-UI s1 Mus musculus cDNA, 3 end/clone = UI-M-BH0-ais-c-07-0-UI/ | P | 309.5 | P | 233.2 | P | 76.9 | A |
| | | clone_end = 3/gb = AI851815/gi = 5495721/ug = Mm 29553/len = 341/STRA = for | | | | | | | |
| 12 | 98073_at | Cluster Incl U66249 Mus musculus Cut alternatively spliced product (CASP) mRNA, partial cds/cds = (0,957)/gb = U66249/ | A | 262.9 | P | 362 | P | 144.8 | A |
| | | gi = 1518859/ug = Mm.34465/len = 1757/STRA = for | | | | | | | |
| 12 | 101524_at | Cluster Incl AI851595 UI-M-BH0-aki-h-08-0-UI.s1 Mus musculus cDNA, 3 end/clone = UI-M-BH0-aki-h-08-0-UI/ | P | 55.9 | P | 50.7 | P | 8.6 | A |
| | | clone_end = 3/gb = AI851595/gi = 5495501/ug = Mm 86187/len = 521/STRA = for | | | | | | | |
| 12 | 102982_at | Cluster Incl AI854581 UI-M-BH0-ake-a-06-0-UI.s1 Mus musculus cDNA, 3 end/clone = UI-M-BH0-ake-a-06-0-UI/ | P | 304.8 | P | 214.1 | P | 196.4 | A |
| | | clone_end = 3/gb = AI854581/gi = 5498487/ug = Mm 1511/len = 562/STRA = for | | | | | | | |
| 12 | 96148_at | Cluster Incl AI844238 UI-M-AL1-ahk-f-10-0-UI s1 Mus musculus cDNA, 3 end/clone = UI-M-AL1-ahk-f-10-0-UI/ | P | 179.7 | P | 100.8 | P | 141.4 | M |
| | | clone_end = 3/gb = AI844238/gi = 5488144/ug = Mm 28232/len = 395/STRA = rev | | | | | | | |
| 12 | 96165_at | Cluster Incl AW125109 UI-M-BH2.1-apx-h-05-0-UI s1 Mus musculus cDNA, 3 end/clone = UI-M-BH2 1-apx-h-05-0-UI/ | P | 436.9 | P | 430.2 | P | 160 | A |
| | | clone_end = 3/gb = AW125109/gi = 6100639/ug = Mm.28370/len = 376/STRA = rev | | | | | | | |
| 12 | 99923_at | Cluster Incl AA615429: vo78b10.r1 Mus musculus cDNA, 5 end/clone = IMAGE-1065211/clone_end = 5/gb = AA615429/ | P | 116.2 | P | 98.9 | A | 33.2 | P |
| | | gi = 2502657/ug = Mm.43157/len = 375/STRA = for | | | | | | | |
| 12 | 104418_at | Cluster Incl AA796868: vp19h01.r1 Mus musculus cDNA, 5 end/clone = IMAGE-1069105/clone_end = 5/gb = AA796868/ | P | 58 | P | 100.2 | P | 65.1 | P |
| | | gi = 2859823/ug = Mm 28920/len = 343/STRA = for | | | | | | | |
| 12 | 93175_at | Cluster Incl AI049206 ub39g05 r1 Mus musculus cDNA, 5 end/clone = IMAGE-1380152/clone_end = 5/gb = AI049206/ | P | 269.9 | P | 218.2 | P | 215 | A |
| | | gi = 3297493/ug = Mm 5357/len = 501/STRA = for | | | | | | | |
| 12 | 92806_at | Cluster Incl AI843868 UI-M-AK1-aeu-d-04-0-UI.s1 Mus musculus cDNA, 3 end/clone = UI-M-AK1-aeu-d-04-0-UI/ | P | 508.3 | P | 528 | P | 424.7 | A |
| | | clone_end = 3/gb = AI843868/gi = 5478081/ug = Mm 12746/len = 408/STRA = for | | | | | | | |
| 12 | 93569_f_at | Cluster Incl AI853444 UI-M-BH0-ajy-d-09-0-UI s1 Mus musculus cDNA, 3 end/clone = UI-M-BH0-ajy-d-09-0-UI/ | A | 71.8 | P | 40.6 | P | 41.3 | A |
| | | clone_end = 3/gb = AI853444/gi = 5497350/ug = Mm.204/len = 363/STRA = for | | | | | | | |
| 12 | 94239_at | Cluster Incl Y08701 M. musculus mRNA for Pinin protein/cds = (18,2195)/gb = Y08701/gi = 2570048/ug = Mm 22347/ | A | 207 | P | 144.8 | P | 36.3 | A |
| | | len = 2813/STRA = for | | | | | | | |
| 12 | 98944_at | Cluster Incl AI848343: UI-M-AH1-ago-c-12-0-U1.s1 Mus musculus cDNA, 3 end/clone = UI-M-AH1-ago-c-12-0-UI/ | P | 314.8 | P | 158.7 | A | 75.5 | A |
| | | clone_end = 3/gb = AI848343/gi = 5492249/ug = Mm.28704/len = 491/STRA = for | | | | | | | |
| 12 | 103346_at | Cluster Incl AF033564: Mus musculus cdc2/CDC28-like kinase 2 (Clk2) mRNA, complete cds/cds = (24,1523)/ | M | 313.1 | P | 231.1 | P | 320 | A |
| | | gb = AF033564/gi = 2645851/ug = Mm 18268/len = 1538/STRA = rev | | | | | | | |
| 12 | 104088_at | Cluster Incl AW060311 UI-M-BH1-amv-d-09-0-UI s1 Mus musculus cDNA, 3 end/clone = UI-M-BH1-amv-d-09-0-UI/ | P | 249.9 | P | 290 | A | 189.5 | A |
| | | clone_end = 3/gb = AW060311/gi = 6008027/ug = Mm.21795/len = 443/STRA = for | | | | | | | |
| 12 | 104112_at | Cluster Incl AI854033 UI-M-BH0-ajm-c-10-0-UI.s1 Mus musculus cDNA, 3 end/clone = UI-M-BH0-ajm-c-10-0-UI/ | P | 299.9 | P | 230.1 | P | 107.1 | A |
| | | clone_end = 3/gb = AI854033/gi = 5497939/ug = Mm.21936/len = 468/STRA = for | | | | | | | |
| 12 | 100988_at | Cluster Incl AA796690 vp15a12 rt Mus musculus cDNA, 5 end/clone = IMAGE-1068670/clone_end = 5/gb = AA796690/ | P | 220.1 | P | 161.8 | P | 167.4 | A |
| | | gi = 2859645/ug = Mm 6807/len = 403/STRA = for | | | | | | | |
| 12 | 102678_at | Cluster Incl L27990 Mus musculus Ro protein mRNA, complete cds/cds = (140,1552)/gb = L27990/gi = 825145 ug = Mm 1384/ | P | 260.2 | P | 195.3 | A | 233.2 | M |
| | | len = 1840/STRA = for | | | | | | | |
| 12 | 104216_at | Cluster Incl AF068921 Mus musculus Ras-binding protein SUR-8 mRNA, complete cds/cds = (0,1748)/gb = AF068921/ | P | 101.8 | P | 143.2 | P | 107.9 | P |
| | | gi = 3252980/ug = Mm 27860/len = 1749/STRA = for | | | | | | | |
| 12 | 93112_at | Cluster Incl D86725 Mouse mRNA for mMCM2, complete cds/cds = (50,2764)/gb = D86725/gi = 2381484/ug = Mm 16711/ | P | 239.8 | P | 192.2 | A | 79.5 | A |
| | | len = 3290/STRA = for | | | | | | | |
| 12 | 94829_at | Cluster Incl AW121838 UI-M-BH2 3-anx-h-10-0-UI s2 Mus musculus cDNA, 3 end/clone = UI-M-BH2 3-anx-h-10-0-UI/ | A | 276.6 | P | 226.4 | M | 236 | A |
| | | clone_end = 3/gb = AW121838/gi = 6097212/ug = Mm 22746/len = 319/STRA = for | | | | | | | |
| 12 | 97470_at | Cluster Incl D86424 Mus musculus mRNA for high-sulfur keratin protein, partial cds/cds = (0,291)/gb = D86424/gi = 1944400/ | P | 495.9 | P | 879 | P | 45 | A |
| | | ug = Mm 30539/len = 409/STRA = for | | | | | | | |
| 12 | 98973_at | Cluster Incl AA982595: uh10e10 r1 Mus musculus cDNA, 5 end/clone = IMAGE-1617546/clone_end = 5/gb = AA982595/ | P | 235.5 | P | 237.3 | P | 156.4 | A |
| | | gi = 3161254/ug = Mm 36583/len = 407/STRA = for | | | | | | | |
| 12 | 100947_at | Cluster Incl AI847906: UI-M-AI1-afl-a-11-0-UI s1 Mus musculus cDNA, 3 end/clone = UI-M-AI1-afl-a-11-0-UI/clone_end = 3/ | P | 133.8 | P | 102.6 | P | 140.7 | M |
| | | gb = AI847906/gi = 5491812/ug = Mm 640/len = 510/STRA = for | | | | | | | |
| 12 | 103957_at | Cluster Incl X57349 M musculus mRNA for transfemn receptor/cds = (0,2291)/gb = X57349/gi = 54914/ug = Mm 25560/ | P | 83.6 | P | 80.7 | A | 49.3 | A |
| | | len = 2292/STRA = for | | | | | | | |
| 12 | 104427_at | Cluster Incl AA930526: vo45f08 r1 Mus musculus cDNA, 5 end/clone = IMAGE-1052871/clone_end = 5/gb = AA930526/ | P | 384 | P | 309.2 | A | 254.3 | A |
| | | gi = 3080016/ug = Mm 29172/len = 410/STRA = for | | | | | | | |
| 12 | 104468_at | Cluster lncl AW045530: UI-M-BH1-aks-c-05-0-UI s1 Mus musculus cDNA, 3 end/clone = UI-M-BH1-aks-c-05-0-UI/ | P | 92 | P | 114.1 | A | 76.5 | A |
| | | clone_end = 3/gb = AW045530/gi = 5906059/ug = Mm 29755/len = 391/STRA = for | | | | | | | |
| 12 | 104694_at | Cluster Incl AI152353: ud79d01.r1 Mus musculus cDNA, 5 end/clone = IMAGE-1477057/clone_end = 5/gb = AI152353/ | P | 252.5 | P | 186.7 | P | 158.9 | A |
| | | gi = 3680822/ug = Mm 3347/len = 386/STRA = for | | | | | | | |
| 12 | 102102 _at | Cluster Incl C80410 C80410 Mus musculus cDNA, 3 end/clone = J0081D08/clone_end = 3/gb = C80410/gi = 2520740/ | A | 103.5 | A | 87.1 | P | 57.1 | M |
| | | ug = Mm 42447/len = 560/STRA = rev | | | | | | | |
| 12 | 95655_at | Cluster Incl AA717740 vt98b12 r1 Mus musculus cDNA, 5 end/clone = IMAGE-1179187/clone_end = 5/gb = AA717740/ | P | 80.3 | P | 38.6 | A | −3.5 | A |
| | | gi = 2730014/ug = Mm 29540/len = 283/STRA = for | | | | | | | |
| 12 | 99570_s_at | Cluster Incl AF029982 Mus musculus sarco(endo)plasmic reticulum calcium ATPase (SERCA2) gene, promoter region, | M | −8.8 | P | −13.8 | A | −122.1 | P |
| | | exons 1-3, and partial cds/cds = (523,741)/gb = AF029982/gi = 4103931/ug = Mm 42255/len = 742/STRA = for | | | | | | | |
| 12 | 100565_at | Cluster Incl AW123396 UI-M-BH2.1-apf-h-12-0-UI s1 Mus musculus cDNA, 3 end/clone = UI-M-BH2.1-apf-h-12-0-UI/ | M | 175.9 | P | 156.6 | P | 126 | A |
| | | clone_end = 3/gb = AW123396/gi = 6098926/ug = Mm 5736/len = 420/STRA = for | | | | | | | |
| 12 | 101515_at | Cluster Incl AF006688: Mus musculus peroxisomal acyl-CoA oxidase (muspaox) mRNA, complete cds/cds = (80,2065)/ | P | 203.6 | P | 142.4 | A | 175.9 | A |
| | | gb = AF006688/gi = 2253379/ug = Mm 8403/len = 3778/STRA = for | | | | | | | |
| 12 | 102004_at | Cluster Incl AI530403 ui92f02.y1 Mus musculus cDNA, 5 end/clone = IMAGE-1889883/clone_end = 5/gb = AI530403/ | P | 25.7 | P | −49.5 | M | 35.7 | A |
| | | gi = 4444538/ug = Mm 99132/len = 906/STRA = for | | | | | | | |
| 12 | 103033_at | Cluster Incl X06454 Mouse mRNA for sex-limited protein Slp(w7) alpha-gamma chain/cds = (0,1784)/gb = X06454/gi = 54105/ | P | 267.7 | P | 186 | A | 119.8 | A |
| | | ug = Mm 16106/len = 1893/STRA = for | | | | | | | |
| 12 | 103427_at | Cluster Incl AW123223 UI-M-BH2.1-apg-e-07-0-UI s1 Mus musculus cDNA, 3 end/clone = UI-M-BH2 1-apg-e-07-0-UI/ | A | 268.4 | P | 154.8 | A | 158.9 | A |
| | | clone_end = 3/gb = AW123223/gi = 6098793/ug = Mm.18968/len = 410/STRA = for | | | | | | | |
| 12 | 103658_r_at | Cluster Incl AI836552 UI-M-AP0-abe-e-08-0-UI.s1 Mus musculus cDNA, 3 end/clone = UI-M-AP0-abe-e-08-0-UI/ | A | 115.5 | P | 94 | P | 87.1 | A |
| | | clone_end = 3/gb = AI836552/gi = 5470765/ug = Mm.20559/len = 417/STRA = for | | | | | | | |
| 12 | 100925_at | Cluster Incl AI131982 uc36a02 r1 Mus musculus cDNA, 5 end/clone = IMAGE-1400042/clone_end = 5/gb = AI131982/ | P | 79.8 | P | 32.2 | M | 26.7 | A |
| | | gi = 3601998/ug = Mm.608/len = 398/STRA = for | | | | | | | |
| 12 | 103963_f_at | Cluster Incl AA914345: vy94a08 r1 Mus musculus cDNA, 5 end/clone = IMAGE-1313846/clone_end = 5/gb = AA914345/ | P | 147.5 | P | −2.3 | A | 22.5 | A |
| | | gi = 3053737/ug = Mm.25624/len = 452/STRA = for | | | | | | | |
| 12 | 92338_f_at | Cluster Incl AI957030 ul74a01 x1 Mus musculus cDNA, 3 end/clone = IMAGE-2136264/clone_end = 3/gb = AI957030/ | A | 84.1 | P | 36.1 | M | 1.1 | A |
| | | gi = 5749739/ug = Mm 39024/len = 550/STRA = for | | | | | | | |
| 12 | 100389_at | Cluster Incl AF021231 Mus musculus acetylcholinesterase-associated collagen (COIQ) gene/cds = (0,710)/gb = AF021231/ | P | 418.2 | M | 477.7 | M | 355.8 | A |
| | | gi = 2460054/ug = Mm 89475/len = 711/STRA = for | | | | | | | |
| 13 | 92578_at | Cluster Incl U10118 Mus musculus endothelial-monocyte activating polypeptide II mRNA, complete cds/cds = (63,995)/ | P | 906.4 | P | 891.2 | P | 751 | P |
| | | gb = U10118/gi = 498911/ug = Mm.10507/len = 1068/STRA = for | | | | | | | |
| 13 | 92633_at | Cluster Incl AJ242663 Mus musculus mRNA for cathepsin Z precursor (ctsZ gene)/cds = (104,1024)/gb = AJ242663/ | P | 433.1 | P | 313.7 | P | 186 | P |
| | | gi = 5019547/ug = Mm 115/len = 1291/STRA = for | | | | | | | |
| 13 | 92794_f_at | Cluster Incl M35970 Mouse tumor metastatic process-associated protein (NM23) gene mRNA, 3 end/cds = (0,500)/ | P | 250.1 | P | 201.1 | P | 89.2 | P |
| | | gb = M35970/gi = 200066/ug = Mm 1260/len = 664/STRA = for | | | | | | | |
| 13 | 92800_i_at | Cluster Incl AI836694: UI-M-AJ0-aax-a-08-0-UI s1 Mus musculus cDNA, 3 end/clone = UI-M-AJ0-aax-a-08-0-UI/ | P | 2883.7 | P | 3762.8 | P | 1542.1 | P |
| | | clone_end = 3/gb = AI836694/gi = 5470907/ug = Mm 12677/len = 344/STRA = for | | | | | | | |
| 13 | 92804_at | Cluster Incl AW122864 UI-M-BH2 1-apa-a-02-0-UI s1 Mus musculus cDNA, 3 end/clone = UI-M-BH2 1-apa-a-02-0-UI/ | P | 390.5 | P | 342.5 | P | 271 | P |
| | | clone_end = 3/gb = AW122864/gi = 6098394/ug = Mm 12706/len = 346/STRA = for | | | | | | | |
| 13 | 92807_at | Cluster Incl X77585: Thioredoxin/cds = (74,391)/gb = X77585/gi = 453971/ug = Mm 1275/len = 538/STRA = for | P | 2931 | P | 3426.8 | P | 2132.2 | P |
| 13 | 92834_at | Cluster Incl X51528: Tissue specific transplantation antigen P198-7/cds = (18,629)/gb = X51528/gi = 55049/ug = Mm.13020/ | P | 804.4 | P | 1149.1 | P | 793.2 | P |
| | | len = 654/STRA = for | | | | | | | |
| 13 | 92848_at | Cluster Incl X64837: M. musculus Oat mRNA for omithine aminotransferase/cds = (114,1433)/gb = X64837/gi = 53458/ | P | 874.4 | P | 766.1 | P | 593.9 | P |
| | | ug = Mm.13694/len = 2125/STRA = for | | | | | | | |
| 13 | 92884_at | Cluster Incl AW045468: UI-M-BH1-akr-b-07-0-UI s1 Mus musculus cDNA, 3 end/clone = UI-M-BH1-akr-b-07-0-UI/ | P | 2458.9 | P | 2621.1 | P | 2340.3 | P |
| | | clone_end = 3/gb = AW045468/gi = 5905962/ug = Mm.14534/len = 318/STRA = for | | | | | | | |
| 13 | 93008_at | Cluster Incl AW120557 UI-M-BH1-ana-f-03-0-UI s2 Mus musculus cDNA, 3 end/clone = UI-M-BH1-ana-f-03-0-UI/ | P | 668.8 | P | 728.3 | P | 454.2 | P |
| | | clone_end = 3/gb = AW120557/gi = 6095890/ug = Mm 14585/len = 530/STRA = for | | | | | | | |
| 13 | 93019_at | Cluster Incl Z35401 M musculus C3H gene for histone H2A.X/cds = (0,431)/gb = Z35401/gi = 515878/ug = Mm 14767/ | P | 1171.8 | P | 1640.6 | P | 842.8 | P |
| | | len = 432/STRA = for | | | | | | | |
| 13 | 93254_at | Cluster Incl AI839142 UI-M-AO0-ach-d-11-0-UI s1 Mus musculus cDNA, 3 end/clone = UI-M-AO0-ach-d-11-0-UI/ | P | 1470.4 | P | 1393.7 | P | 1291.1 | P |
| | | clone_end = 3/gb = AI839142/gi = 5473355/ug = Mm 1700/len = 523/STRA = for | | | | | | | |
| 13 | 93257_at | Cluster Incl AW048287 UI-M-BH1-aly-f-10-0-UI s1 Mus musculus cDNA, 3 end/clone = UI-M-BH1-aly-f-10-0-UI/ | P | 600.5 | P | 648.7 | P | 449.7 | P |
| | | clone_end = 3/gb = AW048287/gi = 5908816/ug = Mm 1706/len = 402/STRA = for | | | | | | | |
| 13 | 93281_at | Cluster Incl AF049125: Mus musculus taipoxin-associated calcium binding protein 49 mRNA, complete cds/cds = (193,1158)/ | P | 484.4 | P | 328.2 | P | 105.1 | P |
| | | gb = AF049125/gi = 2935465/ug = Mm 1782/len = 1790/STRA = for | | | | | | | |
| 13 | 93298_at | Cluster Incl U34883 3-phosphoadenosine 5-phosphosulfate synthase 1/cds = (33,1907)/gb = U34883/gi = 1109675/ | P | 539.7 | P | 525.4 | P | 382.3 | P |
| | | ug = Mm 18161/len = 2479/STRA = for | | | | | | | |
| 13 | 93326_at | Cluster Incl D26483: Mouse mRNA for PE31/TALLA, complete cds/cds = (5,739)/gb = D26483/gi = 685220/ug = Mm 18590/ | P | 463.2 | P | 478.4 | P | 336.4 | P |
| | | len = 1720/STRA = for | | | | | | | |
| 13 | 93531_at | Cluster Incl AI853855: UI-M-BH0-ajr-e-09-0-UI s1 Mus musculus cDNA, 3 end/clone = UI-M-BH0-ajr-e-09-0-UI/ | P | 775.9 | P | 883.6 | P | 546.2 | P |
| | | clone_end = 3/gb = AI853855/gi = 5497761/ug = Mm 19834/len = 540/STRA = for | | | | | | | |
| 13 | 93536_at | Cluster Incl L22472: Bcl2-associated X protein/cds = (0,578)/gb = L22472/gi = 388191/ug = Mm 19904/len = 579/STRA = for | P | 332.6 | P | 359.9 | P | 297.1 | P |
| 13 | 93548_at | Cluster Incl AW122942: UI-M-BH2.1-aoz-a-11-0-UI s2 Mus musculus cDNA, 3 end/clone = UI-M-BH2 1-aoz-a-11-0-UI/ | P | 867.4 | P | 1065.2 | P | 543.9 | P |
| | | clone_end = 3/gb = AW122942/gi = 6098472/ug = Mm 20188/len = 540/STRA = for | | | | | | | |
| 13 | 93551_at | Cluster Incl AW121424: UI-M-BH2.2-aon-b-07-0-UI s1 Mus musculus cDNA, 3 end/clone = UI-M-BH2 2-aon-b-07-0-UI/ | P | 1541.9 | P | 1760 | P | 1084 | P |
| | | clone_end = 3/gb = AW121424/gi = 6096757/ug = Mm 20207/len = 418/STRA = for | | | | | | | |
| 13 | 93559_at | Cluster Incl D90374: Mouse mRNA for APEX nuclease, complete cds/cds = (66,1019)/gb = D90374/gi = 220336/ug = Mm 203/ | P | 781 | P | 668.5 | P | 586 | P |
| | | len = 1185/STRA = for | | | | | | | |
| 13 | 93564_at | Cluster Incl AW122542: UI-M-BH2 2-aox-c-12-0-UI s1 Mus musculus cDNA, 3 end/clone = UI-M-BH2 2-aox-c-12-0-UI/ | P | 564.8 | P | 655.4 | P | 428.8 | P |
| | | clone_end = 3/gb = AW122542/gi = 6098066/ug = Mm 20353/len = 471/STRA = for | | | | | | | |
| 13 | 93595_at | Cluster Incl AF111172: Mus musculus lysosomal pepstatin insensitive protease precursor (Cln2) mRNA, complete cds/ | P | 744.1 | P | 856.3 | P | 419.1 | P |
| | | cds = (28,1716)/gb = AF111172/gi = 4106912/ug = Mm 20837/len = 2270/STRA = for | | | | | | | |
| 13 | 93599_at | Cluster Incl AI841260: UI-M-AM0-adu-d-05-0-UI s1 Mus musculus cDNA, 3 end/clone = UI-M-AM0-adu-d-05-0-UI/ | P | 960.2 | P | 1146.5 | P | 534.1 | P |
| | | clone_end = 3/gb = AI841260/gi = 5475478/ug = Mm 20877/len = 510/STRA = for | | | | | | | |
| 13 | 93815_at | Cluster Incl AI839425: UI-M-AN0-aco-e-06-0-UI s1 Mus musculus cDNA, 3 end/clone = UI-M-AN0-aco-e-06-0-UI/ | P | 1256.8 | P | 1401.5 | P | 1072.6 | P |
| | | clone_end = 3/gb = AI839425/gi = 5473638/uq = Mm 21501/len = 402/STRA = for | | | | | | | |
| 13 | 93833_s_at | Cluster Incl X05862: Mouse H2B and H2A histone genes (291A)/cds = (0,380)/gb = X05862/gi = 51302/ug = Mm 21579/ | P | 501.2 | P | 450.3 | P | 298.3 | P |
| | | len = 381/STRA = for | | | | | | | |
| 13 | 93982_at | Cluster Incl AW122476: UI-M-BH2.2-aou-e-01-0-UI s1 Mus musculus cDNA, 3 end/clone = UI-M-BH2 2-aou-e-01-0-UI/ | P | 626.5 | P | 683 | P | 459.6 | P |
| | | clone_end = 3/gb = AW122476/gi = 6097997/ug = Mm 21707/len = 182/STRA = for | | | | | | | |
| 13 | 94005_at | Cluster Incl AI157548: ue55h05 r1 Mus musculus cDNA, 5 end/clone = IMAGE-1495065/clone_end = 5/gb = AI157548/ | P | 339.1 | P | 364.6 | P | 242.6 | P |
| | | gi = 3686017/ug = Mm 21780/len = 625/STRA = for | | | | | | | |
| 13 | 94017_s_at | Cluster Incl X98511 M musculus PR264 gene/cds = (0,365)/gb = X98511/gi = 1405746/ug = Mm 21841/len = 366/STRA = for | P | 3762.5 | P | 4562.9 | P | 2919.8 | P |
| 13 | 94040_at | Cluster Incl D73368: House mouse; musculus domesticus adult thymus mRNA for Mer protein, complete cds/cds = (41,355)/ | P | 2580.6 | P | 2273.3 | P | 1520.3 | P |
| | | gb = D73368/gi = 1620873/ug = Mm 21952/len = 754/STRA = for | | | | | | | |
| 13 | 94076_i_at | Cluster Incl D31717 Mouse MARib mRNA for ribophorin, complete cds/cds = (41,1864)/gb = D31717/gi = 1468960/ | P | 734.3 | P | 744.5 | P | 506.5 | P |
| | | ug = Mm 22130/len = 2192/STRA = for | | | | | | | |
| 13 | 94077_f_at | Cluster Incl D31717 Mouse MARib mRNA for ribophorin, complete cds/cds = (41,1864)/gb = D31717/gi = 1468960/ | P | 1392 | P | 1619.3 | P | 1122.3 | P |
| | | ug = Mm 22130/len = 2192/STRA = for | | | | | | | |
| 13 | 94209_g_at | Cluster Incl AW045202 UI-M-BH1-alh-08-0-UI.s1 Mus musculus cDNA, 3 end/clone = UI-M-BH1-alh-c-08-0-UI/ | P | 504.7 | P | 448.8 | P | 295.7 | P |
| | | clone_end = 3/gb = AW045202/gi = 5905731/ug = Mm 2220/len = 430/STRA = for | | | | | | | |
| 13 | 94259_at | Cluster Incl AB024935 Mus musculus Sid3177 mRNA, complete cds/cds = (111,593)/gb = AB024935/gi = 5931546/ | P | 919.8 | P | 1144.3 | P | 535 | P |
| | | ug = Mm 22421/len = 1510/STRA = for | | | | | | | |
| 13 | 94273_at | Cluster Incl AI849067: UI-M-AH1-agw-d-04-0-UI s1 Mus musculus cDNA, 3 end/clone = UI-M-AH1-agw-d-04-0-UI/ | P | 629.9 | P | 702.5 | P | 461.2 | P |
| | | clone_end = 3/gb = AI849067/gi = 5492973/ug = Mm 22485/len = 507/STRA = for | | | | | | | |
| 13 | 94292_at | Cluster Incl AF096285 Mus musculus serine-threonine kinase receptor-associated protein mRNA, complete cds/ | P | 637 | P | 579.8 | P | 404.2 | P |
| | | cds = (85,1140)/gb = AF096285/gi = 4063382/ug = Mm.22584/len = 1630/STRA = for | | | | | | | |
| 13 | 94323_at | Cluster Incl AW048132 UI-M-BH1-alq-f-07-0-UI s1 Mus musculus cDNA, 3 end/clone = UI-M-BH1-alq-f-07-0-UI/ | P | 1530.5 | P | 1579.1 | P | 1097.3 | P |
| | | clone_end = 3/gb = AW048132/gi = 5908661/ug = Mm 22667/len = 271/STRA = for | | | | | | | |
| 13 | 95404_at | Cluster Incl AW123453 UI-M-BH2 1-apj-e-11-0-UI.s1 Mus musculus cDNA, 3 end/clone = UI-M-BH2 1-apj-e-11-0-UI/ | P | 328.5 | P | 424.9 | P | 278.2 | P |
| | | clone_end = 3/gb = AW123453/gi = 6098983/ug = Mm 24199/len = 274/STRA = for | | | | | | | |
| 13 | 95445_at | Cluster Incl AW124194 UI-M-BH2 1-aph-b-0I-0-UI s1 Mus musculus cDNA, 3 end/clone = UI-M-BH2.1-aph-b-01-0-UI/ | P | 399.7 | P | 418.3 | P | 209.3 | P |
| | | clone_end = 3/gb = AW124194/gi = 6099724/ug = Mm 24593/len = 341/STRA = for | | | | | | | |
| 13 | 95473_s_at | Cluster Incl AW123338 UI-M-BH2 1-apf-c-12-0-UI s1 Mus musculus cDNA, 3 end/clone = UI-M-BH2 1-apl-c-12-0-UI/ | P | 1002 | P | 1156.3 | P | 633.7 | P |
| | | clone_end = 3/gb = AW123338/gi = 6098868/ug = Mm.24808/len = 425/STRA = for | | | | | | | |
| 13 | 95491_at | Cluster Incl AB015652 Mus musculus gene for DJ-1, complete cds/cds = (0,521)/gb = AB015652/gi = 3256342/ug = Mm 2507/ | P | 1021 | P | 1207.3 | P | 759.4 | P |
| | | len = 522/STRA = for | | | | | | | |
| 13 | 96016_at | Cluster Incl AW045665 UI-M-BH1-alg-h-11-0-UI.s1 Mus musculus cDNA, 3 end/clone = UI-M-BH1-alg-h-11-0-UI/ | P | 634.7 | P | 813.5 | P | 317.6 | P |
| | | clone_end = 3/gb = AW045665/gi = 5906194/ug = Mm.25590/len = 395/STRA = for | | | | | | | |
| 13 | 96024_at | Cluster Incl L32838: Mus musculus (clone C7/B9) S-adenosyl homocysteine hydrolase (ahcy) mRNA, complete cds/ | P | 557.4 | P | 895.9 | P | 586.4 | P |
| | | cds = (5,1303)/gb = L32836/gi = 904131/ug = Mm.2573/len = 2057/STRA = for | | | | | | | |
| 13 | 96025_g_at | Cluster Incl L32836: Mus musculus (clone C7/B9) S-adenosyl homocysteine hydrolase (ahcy) mRNA, complete cds/ | P | 816.3 | P | 1212.9 | P | 722.8 | P |
| | | cds = (5,1303)/gb = L32836/gi = 904131/ug = Mm.2573/len = 2057/STRA = for | | | | | | | |
| 13 | 96632_at | Cluster Incl AB025049: Mus musculus mRNA for Sid393p, complete cds/cds = (96,962)/gb = AB025049/gi = 5931552/ | P | 2285.7 | P | 2212.2 | P | 1875.2 | P |
| | | ug = Mm.27218/len = 1621/STRA = for | | | | | | | |
| 13 | 96635_at | Cluster Incl AW125392 UI-M-BH2 3-aqh-c-08-0-UI s1 Mus musculus cDNA, 3 end/clone = UI-M-BH2 3-aqh-c-08-0-UI/ | P | 203.4 | P | 179.7 | P | 57.4 | P |
| | | clone_end = 3/gb = AW125392/gi = 6100922/ug = Mm 27235/len = 463/STRA = for | | | | | | | |
| 13 | 96696_at | Cluster Incl AI837110 UI-M-AK0-adc-e-02-0-UI s1 Mus musculus cDNA, 3 end/clone = UI-M-AK0-adc-e-02-0-UI/ | P | 1355.4 | P | 1372.2 | P | 1119.1 | P |
| | | clone_end = 3/gb = AI837110/gi = 5471323/ug = Mm.27545/len = 124/STRA = for | | | | | | | |
| 13 | 97247_at | Cluster Incl AW124778 UI-M-BH2.1-aqa-g-10-0-UI s1 Mus musculus cDNA, 3 end/clone = UI-M-BH2 1-aqa-g-10-0-UI/ | P | 803.8 | P | 535 | P | 439.6 | P |
| | | clone_end = 3/gb = AW124778/gi = 6100308/ug = Mm 27847/len = 462/STRA = for | | | | | | | |
| 13 | 97250_at | Cluster Incl AW049326: UI-M-BH1-amr-f-01-0-UI s1 Mus musculus cDNA, 3 end/clone = UI-M-BH1-amr-f-01-0-UI/ | P | 1471.7 | P | 1346.6 | P | 818.4 | P |
| | | clone_end = 3/gb = AW049326/gi = 5909855/ug = Mm 27858/len = 234/STRA = for | | | | | | | |
| 13 | 97309_at | Cluster Incl AW214164 uo41a02.x1 Mus musculus cDNA, 3 end/clone = IMAGE-2645066/clone_end = 3/gb = AW214164/ | P | 495.9 | P | 484.7 | P | 232.2 | P |
| | | gi = 6520606/ug = Mm 28066/len = 253/STRA = for | | | | | | | |
| 13 | 97311_at | Cluster Incl AIB36509: UI-M-AI0-aal-f-06-0-UI.s1 Mus musculus cDNA, 3 end/clone = UI-M-AI0-aal-f-06-0-UI/clone_end = 3/ | P | 1388.1 | P | 1471.4 | P | 1138.1 | P |
| | | gb = AI836509/gi = 5470722/ug = Mm 28077/len = 320/STRA = for | | | | | | | |
| 13 | 97809_at | Cluster Incl AF109906 Mus musculus MHC class III region RD gene, partial cds, Bf, C2, G9A, NG22, G9, HSP70, HSP70, | P | 896.6 | P | 1027.1 | P | 746.1 | P |
| | | HSC70t, and smRNP genes, complete cds, G7A gene, partial cds, and unknown genes/cds = (0,3002)/gb = AF109906/ | | | | | | | |
| | | gi = 3986763/ug = Mm 28155/len = 300/STRA = for | | | | | | | |
| 13 | 97824_at | Cluster Incl AW121031 UI-M-BH2 3-aof-b-04-0-UI s1 Mus musculus cDNA, 3 end/clone = UI-M-BH2 3-aof-b-04-0-UI/ | P | 1439.2 | P | 1445.2 | P | 1034.2 | P |
| | | clone_end = 3/gb = AW121031/gi = 6096364/ug = Mm 28203/len = 396/STRA = for | | | | | | | |
| 13 | 97836_at | Cluster Incl AI843444 UI-M-AQ1-aec-d-07-0-UI.s1 Mus musculus cDNA, 3 end/clone = UI-M-AQ1-aec-d-07-0-UI/ | P | 764.4 | P | 707.3 | P | 446.9 | P |
| | | clone_end = 3/gb = AI843444/gi = 5477657/ug = Mm 28235/len = 315/STRA = for | | | | | | | |
| 13 | 97907_at | Cluster Incl AW049564 UI-M-BH1-anf-g-07-0-UI s1 Mus musculus cDNA, 3 end/clone = UI-M-BH1-anf-g-07-0-UI/ | P | 644.9 | P | 547.1 | P | 342.6 | P |
| | | clone_end = 3/gb = AW049564/gi = 5910093/ug = Mm 28470/len = 426/STRA = for | | | | | | | |
| 13 | 98904_at | Cluster Incl AW061339 UI-M-BH1-anw-g-01-0-UI s1 Mus musculus cDNA, 3 end/clone = UI-M-BH1-anw-g-01-0-UI/ | P | 542 | P | 444.7 | P | 198 | P |
| | | clone_end = 3/gb = AW061339/gi = 6009090/ug = Mm 28579/len = 376/STRA = for | | | | | | | |
| 13 | 98928_at | Cluster Incl AW122820 UI-M-BH2 1-aoz-b-12-0-UI s1 Mus musculus cDNA, 3 end/clone = UI-M-BH2 1-aoz-b-12-0-UI/ | P | 495.7 | P | 512.5 | P | 268.4 | P |
| | | clone_end = 3/gb = AW122820/gi = 6098350/ug = Mm 28652/len = 302/STRA = for | | | | | | | |
| 13 | 98930_at | Cluster Incl AI844701: UI-M-AL1-ahr-h-03-0-UI s1 Mus musculus cDNA, 3 end/clone = UI-M-AL1-ahr-h-03-0-UI/ | P | 579.2 | P | 879.2 | P | 298.4 | P |
| | | clone_end = 3/gb = AI844701/gi = 5488607/ug = Mm 28668/len = 487/STRA = for | | | | | | | |
| 13 | 94455_at | Cluster Incl AA795062 vs09d03 r1 Mus musculus cDNA, 5 end/clone = IMAGE-1137701/clone_end = 5/gb = AA795062/ | P | 816.7 | P | 839.7 | P | 456.8 | P |
| | | gi = 2858017/ug = Mm 28803/len = 536/STRA = for | | | | | | | |
| 13 | 94503_at | Cluster Incl AI842492 UI-M-AM1-afy-h-05-0-UI s1 Mus musculus cDNA, 3 end/clone = UI-M-AM1-afy-h-05-0-UI/ | P | 743.1 | P | 891.2 | P | 487.3 | P |
| | | clone_end = 3/gb = AI842492/gi = 5476705/ug = Mm 28952/len = 259/STRA = for | | | | | | | |
| 13 | 94510_at | Cluster Incl AI853476 UI-M-BH0-ajy-h-09-0-UI s1 Mus musculus cDNA, 3 end/clone = UI-M-BH0-ajy-h-09-0-UI/ | P | 519.4 | P | 516 | P | 316.8 | P |
| | | clone_end = 3/gb = AI853476/gi = 5497382/ug = Mm 28975/len = 537/STRA = for | | | | | | | |
| 13 | 94524_at | Cluster Incl AW123469 UI-M-BH2 1-apj-g-04-0-UI.s1 Mus musculus cDNA, 3 end/clone = UI-M-BH2.1-apj-g-04-0-UI/ | P | 338.7 | P | 325.4 | P | 254.8 | P |
| | | clone_end = 3/gb = AW123469/gi = 6098999/ug = Mm 29028/len = 452/STRA = for | | | | | | | |
| 13 | 94532_at | Cluster Incl U13841.ATPase, H+ transporting lysosomal (vacuolar proton pump) 32 kDa/cds = (0,686)/gb = U13841/ | P | 850.9 | P | 726.5 | P | 738.9 | P |
| | | gi = 1184662/ug = Mm.29045/len = 687/STRA = for | | | | | | | |
| 13 | 95049_at | Cluster Incl AI837853 UI-M-AP0-abk-d-04-0-UI s1 Mus musculus cDNA, 3 end/clone = UI-M-AP0-abk-d-04-0-UI/ | P | 2690.9 | P | 3719.4 | P | 2061 | P |
| | | clone_end = 3/gb = AI837853/gi = 5472066/ug = Mm 29135/len = 429/STRA = for | | | | | | | |
| 13 | 95067_at | Cluster Incl AI837821 UI-M-AK0-adl-h-06-0-UI s1 Mus musculus cDNA, 3 and/clone = UI-M-AK0-adl-h-06-0-UI/ | P | 227.5 | P | 309.3 | P | 169.9 | P |
| | | clone_end = 3/gb = AI837821/gi = 5472034/ug = Mm 29183/len = 468/STRA = for | | | | | | | |
| 13 | 95734_at | Cluster Incl AI852230 UI-M-BH0-ajc-f-07-0-UI s1 Mus musculus cDNA, 3 end/clone = UI-M-BH0-ajc-f-07-0-UI/clone_end = 3/ | P | 531.4 | P | 572.7 | P | 534.1 | P |
| | | gb = AI852230/gi = 5496136/ug = Mm 29746/len = 392/STRA = for | | | | | | | |
| 13 | 95743_at | Cluster Incl AI846078 UI-M-AN1-afj-a-03-0-UI s2 Mus musculus cDNA, 3 end/clone = UI-M-AN1-afj-a-03-0-UI/ | P | 1707.2 | P | 977.1 | P | 851 | P |
| | | clone_end = 3/gb = AI846078/gi = 5490024/ug = Mm.29769/len = 335/STRA = for | | | | | | | |
| 13 | 96242_at | Cluster Incl AW125751 UI-M-BH2 2-aqi-h-05-0-UI s1 Mus musculus cDNA, 3 end/clone = UI-M-BH2.2-aqi-h-05-0-UI/ | P | 267.1 | P | 357.6 | P | 271.3 | P |
| | | clone_end = 3/gb = AW125751/gi = 6101246/ug = Mm 29802/len = 240/STRA = for | | | | | | | |
| 13 | 96261 _at | Cluster Incl AW122694 UI-M-BH2.2-aot-b-04-0-UI s1 Mus musculus cDNA, 3 end/clone = UI-M-BH2 2-aot-b-04-0-UI/ | P | 1403.8 | P | 1006 | P | 611.4 | P |
| | | clone_end = 3/gb = AW122694/gi = 6098224/ug = Mm 29828/len = 456/STRA = for | | | | | | | |
| 13 | 96280_at | Cluster Incl AI851603 UI-M-BH0-aln-a-07-0-UI.s1 Mus musculus cDNA, 3 end/clone = UI-M-BH0-aln-a-07-0-UI/ | P | 1237.5 | P | 1317.4 | P | 824.9 | P |
| | | clone_end = 3/gb = AI851603/gi = 5495509/ug = Mm.29867/len = 415/STRA = for | | | | | | | |
| 13 | 96295_at | Cluster Incl AW122030 UI-M-BH2 2-aoo-b-05-0-UI.s1 Mus musculus cDNA, 3 end/clone = UI-M-BH2.2-aoo-b-05-0-UI/ | P | 1605.6 | P | 1577.8 | P | 1084.8 | P |
| | | clone_end = 3/gb = AW122030/gi = 6097436/ug = Mm 29902/len = 358/STRA = for | | | | | | | |
| 13 | 96318_at | Cluster Incl AW045739-UI-M-BH1-akq-g-08-0-UI s1 Mus musculus cDNA, 3 end/clone = UI-M-BH1-akq-g-08-0-UI/ | P | 393.5 | P | 360.2 | P | 291.9 | P |
| | | clone_end = 3/gb = AW045739/gi = 5906268/ug = Mm 29925/len = 263/STRA = for | | | | | | | |
| 13 | 96352_at | Cluster Incl AI848845 UI-M-AJ1-ahb-d-02-0-UI.s1 Mus musculus cDNA, 3 end/clone = UI-M-AJ1-ahb-d-02-0-UI/ | P | 870.9 | P | 775.3 | P | 714.7 | P |
| | | clone_end = 3/gb = AI848845/gi = 5492751/ug = Mm 30003/len = 402/STRA = for | | | | | | | |
| 13 | 96847_at | Cluster Incl AI845393 UI-M-AO1-aej-a-03-0-UI s1 Mus musculus cDNA, 3 end/clone = UI-M-AO1-aoj-a-03-0-UI/ | P | 969.8 | P | 648.8 | P | 495 | P |
| | | clone_end = 3/gb = AI845393/gi = 5489299/ug = Mm 30028/len = 278/STRA = for | | | | | | | |
| 13 | 96864_at | Cluster Incl AI848770: UI-M-AJ1-ahg-a-08-0-UI s1 Mus musculus cDNA, 3 end/clone = UI-M-AJ1-ahg-a-08-0-UI/ | P | 320.2 | P | 397.9 | P | 220.8 | P |
| | | clone_end = 3/gb = AI848770/gi = 5492676/ug = Mm 30057/len = 383/STRA = for | | | | | | | |
| 13 | 96959_at | Cluster Incl AW210080: ul50f12.y1 Mus musculus cDNA, 5 end/clone = IMAGE-2101775/clone_end = 5/gb = AW210080/ | P | 1586.2 | P | 1482.2 | P | 1099.1 | P |
| | | gi = 6516020/ug = Mm 30233/len = 505/STRA = for | | | | | | | |
| 13 | 97458_at | Cluster Incl AI845935: UI-M-AK1-aex-f-04-0-UI s1 Mus musculus cDNA, 3 end/clone = UI-M-AK1-aex-f-04-0-UI/ | P | 1069.7 | P | 1139.8 | P | 965.9 | P |
| | | clone_end = 3/gb = AI845935/gi = 5489841/ug = Mm 30267/len = 380/STRA = for | | | | | | | |
| 13 | 97472_at | Cluster Incl AJ006341 Mus musculus mRNA for peroxisomal integral membrane protein PMP34/cds = (47,970)/ | P | 817.7 | P | 888.7 | P | 645.2 | P |
| | | gb = AJ006341/gi = 3183980/ug = Mm 306/1en = 1700/STRA = for | | | | | | | |
| 13 | 97477_at | Cluster Incl AW124594: UI-M-BH2.1-apt-g-02-0-UI.s1 Mus musculus cDNA, 3 end/clone = UI-M-BH2.1-apt-g-02-0-UI/ | P | 594.3 | P | 630.5 | P | 330.6 | P |
| | | clone_end = 3/gb = AW124594/gi = 6100124/ug = Mm.30718/len = 407/STRA = for | | | | | | | |
| 13 | 97479_at | Cluster Incl AJ130961: Mus musculus mRNA for ubiquitin-conjugating enzyme/cds = (20,484)/gb = AJ130961/gi = 3928492/ | P | 1085.1 | P | 1363.3 | P | 700.7 | P |
| | | ug = Mm 3074/len = 2597/STRA = for | | | | | | | |
| 13 | 97535_at | Cluster Incl D87661: Mus musculus mRNA for 14-3-3 eta, complete cds/cds = (184,924)/gb = D87661/gi = 1526540/ | P | 717.1 | P | 753 | P | 586.4 | P |
| | | ug = Mm 3308/len = 1098/STRA = for | | | | | | | |
| 13 | 98037_at | Cluster Incl AW122782 UI-M-BH2 1-aoy-d-05-0-UI s1 Mus musculus cDNA, 3 end/clone = UI-M-BH2 1-aoy-d-05-0-UI/ | P | 582.5 | P | 499 | P | 377.1 | P |
| | | clone_end = 3/gb = AW122782/gi = 6098312/ug = Mm 33907/len = 153/STRA = for | | | | | | | |
| 13 | 98107_at | Cluster Incl AW123801 UI-M-BH2 1-apm-e-08-0-UI s1 Mus musculus cDNA, 3 end/clone = UI-M-BH2 1-apm-e-08-0-UI/ | P | 1101.4 | P | 1313.9 | P | 884.3 | P |
| | | clone_end = 3/gb = AW123801/gi = 6099331/ug = Mm.34796/len = 367/STRA = for | | | | | | | |
| 13 | 98129_at | Cluster Incl AI852553 UI-M-BH0-aiw-g-02-0-UI s1 Mus musculus cDNA, 3 end/clone = UI-M-BH0-aiw-g-02-0-UI/ | P | 2992.5 | P | 4005.1 | P | 2423 | P |
| | | clone_end = 3/gb = AI852553/gi = 5496459/ug = Mm 3532/len = 276/STRA = for | | | | | | | |
| 13 | 98533_at | Cluster Incl AI854779 UI-M-BH0-aka-d-08-0-UI s1 Mus musculus cDNA, 3 end/clone = UI-M-BH0-aka-d-08-0-UI/ | P | 1472.3 | P | 993.9 | P | 629 | P |
| | | clone_end = 3/gb = AI854779/gi = 5498685/ug = Mm 35849/len = 428/STRA = for | | | | | | | |
| 13 | 98574_at | Cluster Incl AI835685 UI-M-AJ0-aau-g-09-0-UI s1 Mus musculus cDNA, 3 end/clone = UI-M-AJ0-aau-g-09-0-UI/ | P | 628.3 | P | 643.2 | P | 498.4 | P |
| | | clone_end = 3/gb = AI835685/gi = 5469898/ug = Mm 3757/len = 227/STRA = for | | | | | | | |
| 13 | 98609_at | Cluster Incl AJ250723 Mus musculus mRNA for septin-like protein Sint1 (Sint1 gene)/cds = (177,1181)/gb = AJ250723/ | P | 1297.2 | P | 1519.3 | P | 1239 | P |
| | | gi = 6165418/ug = Mm 38450/len = 2942/STRA = for | | | | | | | |
| 13 | 99106_at | Cluster Incl AF071315 Mus musculus COP9 complex subunit 6 (COPS6) mRNA, complete cds/cds = (48,1022)/ | P | 1197.2 | P | 1640.6 | P | 932.5 | P |
| | | gb = AF071315/gi = 3309171/ug = Mm 3981/len = 1160/STRA = for | | | | | | | |
| 13 | 99113_at | Cluster Incl AF071313 Mus musculus COP9 complex subunit 3 (COPS3) mRNA, complete cds/cds = (43,1314)/ | P | 500.7 | P | 581.3 | P | 450 | P |
| | | gb = AF071313/gi = 3309167/ug = Mm 40/len = 1583/STRA = for | | | | | | | |
| 13 | 99593_at | Cluster Incl AI852355: UI-M-BH0-ajo-c-05-0-UI s1 Mus musculus cDNA, 3 end/clone = UI-M-BH0-ajo-c-05-0-UI/ | P | 1547.1 | P | 1183.3 | P | 1182.3 | P |
| | | clone_end = 3/gb = AI852355/gi = 5496261/ug = Mm 42805/len = 413/STRA = for | | | | | | | |
| 13 | 99596_f_at | Cluster Incl M13963 Guanine nucleotide binding protein, alpha inhibiting 2/cds = (97,1164)/gb = M13963/gi = 193513/ | P | 4663.7 | P | 5857.9 | P | 4100.8 | P |
| | | ug = Mm 4284/len = 1316/STRA = for | | | | | | | |
| 13 | 99598_g_at | Cluster Incl AI841629 UI-M-AO0-acg-c-01-0-UI s1 Mus musculus cDNA, 3 end/clone = UI-M-AO0-acg-c-01-0-UI/ | P | 2028.3 | P | 2462.1 | P | 1690.1 | P |
| | | clone_end = 3/gb = AI841629/gi = 5475842/ug = Mm 4284/len = 436/STRA = for | | | | | | | |
| 13 | 99618_at | Cluster Incl AI853523: UI-M-BH0-ajd-e-03-0-UI s1 Mus musculus cDNA, 3 end/clone = UI-M-BH0-ajd-e-03-0-UI/ | P | 1288.3 | P | 1493.5 | P | 726 | P |
| | | clone_end = 3/gb = AI853523/gi = 5497429/ug = Mm 43162/len = 425/STRA = for | | | | | | | |
| 13 | 99619_at | Cluster Incl AIB47483 UI-M-AI1-afs-g-03-0-UI s1 Mus musculus cDNA, 3 end/clone = UI-M-AI1-afs-g-03-0-UI/clone_end = 3/ | P | 252 | P | 308.5 | P | −51 | P |
| | | gb = AI847483/gi = 5491389/ug = Mm 43212/len = 471/STRA = for | | | | | | | |
| 13 | 99637_at | Cluster Incl AF011450 Procollagen, type XV/cds = (158,4259)/gb = AF011450/gi = 2558824/ug = Mm 4352/len = 5176/ | P | 901.1 | P | 764.3 | P | 497.5 | P |
| | | STRA = for | | | | | | | |
| 13 | 99655_at | Cluster Incl AB025405 Mus musculus mRNA for sid2895p, complete cds/cds = (98,637)/gb = AB025405/gi = 5931558/ | P | 942.3 | P | 716.3 | P | 546.7 | P |
| | | ug = Mm 43760/len = 1113/STRA = for | | | | | | | |
| 13 | 100040_at | Cluster Incl AI843081: UI-M-AK1-aes-b-06-0-UI s1 Mus musculus cDNA, 3 end/clone = UI-M-AK1-aes-b-06-0-UI/ | P | 239.9 | P | 305 | P | 64.9 | P |
| | | clone_end = 3/gb = AI843081/gi = 5477294/ug = Mm 44225/len = 232/STRA = for | | | | | | | |
| 13 | 100050_at | Cluster Incl M31885 Inhibitor of DNA binding 1/cds = (0,532)/gb = M31885/gi = 194090/ug = Mm 444/len = 927/STRA = for | P | 1076.8 | P | 909.7 | P | 851.5 | P |
| 13 | 100062_at | Cluster Incl X62154.Mini chromosome maintenance deficient (S cerevisiae)/cds = (0,2387)/gb = X62154/gi = 53550/ | P | 340.3 | P | 342.2 | P | 171.7 | P |
| | | ug = Mm 4502/len = 2813/STRA = rev | | | | | | | |
| 13 | 100065_r_at | Cluster Incl M63801 Mus musculus connexin 43 (alpha-1 gap junction) mRNA, complete cds/cds = (220,1368)/gb = M63801/ | P | 2440.2 | P | 2785.6 | P | 1559.5 | P |
| | | gi = 191773/ug = Mm 4504/len = 1408/STRA = for | | | | | | | |
| 13 | 100578_at | Cluster Incl M33934 Mouse IMP dehydrogenase mRNA, complete cds/cds = (66,1610)/gb = M33934/gi = 198393/ | P | 1126.8 | P | 1341.3 | P | 912.3 | P |
| | | ug = Mm 6065/len = 1707/STRA = for | | | | | | | |
| 13 | 100628_at | Cluster Incl AI840263 UI-M-AH0-act-e-04-0-UI.s1 Mus musculus cDNA, 3 end/clone = UI-M-AH0-act-e-04-0-UI/ | P | 980.3 | P | 968.1 | P | 762.3 | P |
| | | clone_end = 3/gb = AI840263/gi = 5474476/ug = Mm 666/len = 248/STRA = for | | | | | | | |
| 13 | 101017_at | Cluster Incl AA791962: vs65h09 r1 Mus musculus cDNA, 5 end/clone = IMAGE-1151201/clone_end = 5/gb = AA791962/ | P | 423.7 | P | 548.5 | P | 475.4 | P |
| | | gi = 2854917/ug = Mm 6839/len = 336/STRA = rev | | | | | | | |
| 13 | 101061_at | Cluster Incl AI845293 UI-M-BG0-ahw-f-04-0-UI.s1 Mus musculus cDNA, 3 end/clone = UI-M-BG0-ahw-f-04-0-UI/ | P | 1150 | P | 1193.1 | P | 716.5 | P |
| | | clone_end = 3/gb = AI845293/gi = 5489199/ug = Mm 7091/len = 254/STRA = for | | | | | | | |
| 13 | 101081_at | Cluster Incl AJ010483 Mus musculus mRNA for CtBP1 protein/cds = (99,1421)/gb = AJ010483/gi = 3452506/ug = Mm 7286/ | P | 1287 | P | 1405.8 | P | 1144.7 | P |
| | | len = 2240/STRA = for | | | | | | | |
| 13 | 101104_at | Cluster Incl AB001990: Mus musculus mRNA for Dcra, complete cds/cds = (50,943)/gb = AB001990/gi = 2588992/ | P | 531.9 | P | 496.6 | P | 383.8 | P |
| | | ug = Mm 7472/len = 2157/STRA = for | | | | | | | |
| 13 | 101105_at | Cluster Incl AB025349: Mus musculus L2BP1/BAF mRNA for LAP2 binding protein 1, complete cds/cds = (57,326)/ | P | 1329.1 | P | 1638 | P | 1082.1 | P |
| | | gb = AB025349/gi = 5708070/ug = Mm 7508/len = 603/STRA = for | | | | | | | |
| 13 | 101519_at | Cluster Incl M29264 Mouse signal recognition particle subunit (SRP14) mRNA, complete cds/cds = (7,339)/gb = M29264/ | P | 1158.4 | P | 1041.3 | P | 860 | P |
| | | gi = 201062/ug = Mm.850/len = 706/STRA = for | | | | | | | |
| 13 | 101521_at | Cluster Incl AB013819 Mus musculus mRNA for TIAP, complete cds/cds = (108,530)/gb = AB013819/gi = 3135206/ | P | 508.3 | P | 583.1 | P | 137.5 | P |
| | | ug = Mm.8552/len = 955/STRA = for | | | | | | | |
| 13 | 101964_at | Cluster Incl U05809: Transketolase/cds = (91,1962)/gb = U05809/gi = 452485/ug = Mm.9307/len = 2062/STRA = for | P | 1033.5 | P | 1551 | P | 603.1 | P |
| 13 | 103364_f_at | Cluster Incl AW045256 UI-M-BH1-akn-a-02-0-UI.s1 Mus musculus cDNA, 3 end/clone = UI-M-BH1-akn-a-02-0-UI/ | P | 334.1 | P | 498.4 | P | 196.4 | P |
| | | clone_end = 3/gb = AW045256/gi = 5905785/ug = Mm.18520/len = 324/STRA = for | | | | | | | |
| 13 | 103381_at | Cluster Incl AI838735 UI-M-AO0-acf-c-11-0-UI s1 Mus musculus cDNA, 3 end/clone = UI-M-AO0-acf-c-11-0-UI/ | P | 693.3 | P | 820.9 | P | 673.6 | P |
| | | clone_end = 3/gb = AI838735/gi = 5472948/ug = Mm.18634/len = 413/STRA = for | | | | | | | |
| 13 | 103606_r_at | Cluster Incl AW121438 UI-M-BH2 2-aon-c-10-0-UI.s1 Mus musculus cDNA, 3 end/clone = UI-M-BH2 2-aon-c-10-0-UI/ | P | 447.5 | P | 336.6 | P | 275.6 | P |
| | | clone_end = 3/gb = AW121438/gi = 6096771/ug = Mm.20156/len = 269/STRA = for | | | | | | | |
| 13 | 103620_a_at | Cluster Incl U77714: Survival motor neuron/cds = (0,866)/gb = U77714/gi = 1946332/ug = Mm 2025/len = 1250/STRA = for | P | 409.1 | P | 430.8 | P | 335.1 | P |
| 13 | 103835_f_at | Cluster Incl AF085192: Mus musculus neural visinin-like protein 3 (NVP-3) mRNA, complete cds/cds = (260,841)/ | P | 780.3 | P | 712 | P | 611.2 | P |
| | | gb = AF085192/gi = 3600082/ug = Mm.20937/len = 1565/STRA = for | | | | | | | |
| 13 | 104108_at | Cluster Incl AJ245569: Mus musculus partial mRNA for hypothetical protein (ORF37 DNA)/cds = (218,4009)/gb = AJ245569/ | P | 274 | P | 213.4 | P | 181.3 | P |
| | | gi = 5869933/ug = Mm 21904/len = 4907/STRA = for | | | | | | | |
| 13 | 104277_at | Cluster Incl AI834815: UI-M-AM0-ads-a-05-0-UI.s1 Mus musculus cDNA, 3 end/clone = UI-M-AM0-ads-a-05-0-UI/ | P | 238 | P | 328 | P | 96.1 | P |
| | | clone_end = 3/gb = AI834815/gi = 5468993/ug = Mm 22218/len = 168/STRA = for | | | | | | | |
| 13 | 104333_at | Cluster Incl U69488 Mus musculus viral envelope like protein (G7e) gene, complete cds/cds = (154,1944)/gb = U69488/ | P | 569.3 | P | 571 | P | 319.1 | P |
| | | gi = 2988386/ug = Mm 22506/len = 2910/STRA = for | | | | | | | |
| 13 | 96232_at | Cluster Incl AI854281: UI-M-BH0-ajs-d-09-0-UI s1 Mus musculus cDNA, 3 end/clone = UI-M-BH0-ajs-d-09-0-UI/ | P | 376.3 | P | 406.7 | P | 226.5 | P |
| | | clone_end = 3/gb = AI854281/gi = 5498187/ug = Mm.28776/len = 440/STRA = for | | | | | | | |
| 13 | 97374_at | Cluster Incl AI840458 UI-M-AN0-aci-c-06-0-UI s1 Mus musculus cDNA, 3 end/clone = UI-M-AN0-aci-c-06-0-UI/ | P | 381.1 | P | 430.2 | P | 308.4 | P |
| | | clone_end = 3/gb = AI840458/gi = 5474671/ug = Mm 29721/len = 430/STRA = for | | | | | | | |
| 13 | 97401_at | Cluster Incl AW124244 UI-M-BH2 1-aph-f-05-0-UI s1 Mus musculus cDNA, 3 end/clone = UI-M-BH2 1-aph-f-05-0-UI/ | P | 447.1 | P | 562.7 | P | 420.5 | P |
| | | clone_end = 3/gb = AW124244/gi = 6099774/ug = Mm.29878/len = 381/STRA = for | | | | | | | |
| 13 | 97436_at | Cluster Incl AA869653: vq20g06 r1 Mus musculus cDNA, 5 end/clone = IMAGE-1094842/clone_end = 5/gb = AA869653/ | P | 232.6 | P | 237.6 | P | 66.9 | P |
| | | gi = 2965098/ug = Mm 30110/len = 240/STRA = for | | | | | | | |
| 13 | 98459_at | Cluster Incl AA913994: vy91d09.r1 Mus musculus cDNA, 5 end/clone = IMAGE-1313585/clone_end = 5/gb = AA913994/ | P | 246 | P | 301.1 | P | 217.8 | P |
| | | gi = 3053386/ug = Mm 34759/len = 511/STRA = for | | | | | | | |
| 13 | 98993_at | Cluster Incl U59418 Mus musculus protein phosphatase 2A B alpha3 regulatory subunit mRNA, partial cds/cds = (406,1671)/ | P | 823.7 | P | 1040.5 | P | 735 | P |
| | | gb = U59418/gi = 2429084/ug = Mm 3785/len = 1796/STRA = for | | | | | | | |
| 13 | 99546_at | Cluster Incl M77831: Mouse binding protein 13 (Fkbp13) gene exons 1-5, complete cds/cds = (155,577)/gb = M77831/ | P | 786.3 | P | 965.2 | P | 458.3 | P |
| | | gi = 433782/ug = Mm.4234/len = 740/STRA = for | | | | | | | |
| 13 | 100885_at | Cluster Incl AF013166: Mus musculus serine/threonine kinase (nek2) mRNA, complete cds/cds = (57,1388)/gb = AF013166/ | P | 412.2 | P | 458.8 | P | 365.5 | P |
| | | gi = 2331225/ug = Mm 5379/len = 2869/STRA = for | | | | | | | |
| 13 | 101385_at | Cluster Incl AW047312 UI-M-BH1-amh-f-09-0-UI.s1 Mus musculus cDNA, 3 end/clone = UI-M-BH1-amh-f-09-0-UI/ | P | 685.9 | P | 1224.3 | P | 434.2 | P |
| | | clone_end = 3/gb = AW047312/gi = 5907841/ug = Mm.7142/len = 493/STRA = for | | | | | | | |
| 13 | 102879_s_at | Cluster Incl M31314 Fc receptor, IgG, high affinity I/cds = (1,1215)/gb = M31314/gi = 200752/ug = Mm.150/len = 1279/ | P | 982.5 | P | 843.5 | P | 369 | P |
| | | STRA = for | | | | | | | |
| 13 | 103203_f_at | Cluster Incl AW259499 up28d02.x1 Mus musculus cDNA, 3 end/clone = IMAGE-2655651/clone_end = 3/gb = AW259499/ | P | 276.5 | P | 219.1 | P | 127.8 | P |
| | | gi = 6632417/ug = Mm.1911/len = 454/STRA = for | | | | | | | |
| 13 | 103442_at | Cluster Incl AI843399 UI-M-AQ1-adz-g-11-0-UI s1 Mus musculus cDNA, 3 end/clone = UI-M-AQ1-adz-g-11-0-UI/ | P | 346.8 | P | 445.9 | P | 199.3 | P |
| | | clone_end = 3/gb = AI843399/gi = 5477612/ug = Mm 21475/len = 471/STRA = for | | | | | | | |
| 13 | 104491_at | Cluster Incl AI509330 mt94e09.y1 Mus musculus cDNA, 5 end/clone = IMAGE-637576/clone_end = 5/gb = AI509330/ | P | 742.5 | P | 744.9 | P | 505.6 | P |
| | | gi = 4408235/ug = Mm.3028/len = 360/STRA = for | | | | | | | |
| 13 | 92304_at | Cluster Incl D31863 Phosphatidylinositol glycan, class A/cds = (62,1519)/gb = D31863/gi = 1402591/ug = Mm 3781/len = 3534/ | P | 135.4 | P | 186.8 | P | 74.7 | P |
| | | STRA = for | | | | | | | |
| 13 | 92428_at | Cluster Incl AF039405: Mus musculus arsenite-translocating ATPase (ArsA) mRNA, complete cds/cds = (0,1052)/ | P | 456.2 | P | 496.6 | P | 279.6 | P |
| | | gb = AF039405/gi = 2745699/ug = Mm 41475/len = 1053/STRA = for | | | | | | | |
| 13 | 93670_at | Cluster Incl AW048233 UI-M-BH1-amc-h-10-0-UI s1 Mus musculus cDNA, 3 end/clone = UI-M-BH1-amc-h-10-0-UI/ | P | 536.6 | P | 610.1 | P | 423.9 | P |
| | | clone_end = 3/gb = AW048233/gi = 5908762/ug = Mm 8068/len = 429/STRA = for | | | | | | | |
| 13 | 94788_f_at | Cluster Incl X04663 Mouse mRNA for beta-tubulin (lsotype Mbeta 5)/cds = (78,1412)/gb = X04663/gi = 55047/ug = Mm 1703/ | P | 3524.6 | P | 4404.9 | P | 3414.6 | P |
| | | len = 1591/STRA = for | | | | | | | |
| 13 | 97682_r_at | Cluster Incl J03953 Mouse, glutathione transferase GT9 3 mRNA, 3 end/cds = (0,630)/gb = J03953/gi = 193689/ | P | 1124.5 | P | 1707.1 | P | 564.7 | P |
| | | ug = Mm 37199/len = 1090/STRA = for | | | | | | | |
| 13 | 97758_at | Cluster Incl AB023564 Mus musculus gene for type I peroxiredoxin/cds = (11,610)/gb = AB023564/gi = 6467220/ug = Mm 412/ | P | 3203 | P | 3237.7 | P | 2286.1 | P |
| | | len = 864/STRA = for | | | | | | | |
| 13 | 95791_s_at | Cluster Incl U14648 Splicing factor, arginine/serine-rich 10/cds = (0,853)/gb = U14648/gi = 555923/ug = Mm 87815/len = 1772/ | P | 877.5 | P | 585.8 | P | 294.5 | P |
| | | STRA = for | | | | | | | |
| 13 | 97055_s_at | Cluster Incl AI118194: ue34e03 x1 Mus musculus cDNA, 3 end/clone = IMAGE-1482268/clone_end = 3/gb = AI118194/ | P | 3415.4 | P | 3909 | P | 3449.1 | P |
| | | gi = 3518518/ug = Mm 89213/len = 567/STRA = rev | | | | | | | |
| 13 | 97061_g_at | Cluster Incl AW215489: up04f11.x1 Mus musculus cDNA, 3 end/clone = IMAGE-2651085/clone_end = 3/gb = AW215489/ | P | 3446.3 | P | 4411.6 | P | 2938.1 | P |
| | | gi = 6526165/ug = Mm 89290/len = 522/STRA = rev | | | | | | | |
| 13 | 99678_f_at | Cluster Incl AU018994: AU018994 Mus musculus cDNA, 3 end/clone = J0511B11/clone_end = 3/gb = AU018994/ | P | 822.2 | P | 951.6 | P | 450.3 | P |
| | | gi = 3374497/ug = Mm 95645/len = 265/STRA = rev | | | | | | | |
| 13 | 102476_f_at | Cluster Incl AW121303: UI-M-BH2 3-aog-g-01-0-UI s1 Mus musculus cDNA, 3 end/clone = UI-M-BH2.3-aog-g-01-0-UI/ | P | 739.3 | P | 746 | P | 596.6 | P |
| | | clone_end = 3/gb = AW121303/gi = 6096636/ug = Mm48675/len = 217/STRA = rev | | | | | | | |
| 13 | 93016_at | Cluster Incl U56243 Mus musculus testis 14-3-3 protein theta-subtype mRNA, complete cds/cds = (20,757)/gb = U56243/ | P | 502 | P | 534.5 | P | 341.5 | P |
| | | gi = 1777958/ug = Mm 14722/len = 761/STRA = for | | | | | | | |
| 13 | 93993_at | Cluster Incl AI851062: UI-M-BH0-ajv-h-01-0-UI s1 Mus musculus cDNA, 3 end/clone = UI-M-BH0-ajv-h-01-0-UI/ | P | 405.6 | P | 369.2 | P | 275.9 | A |
| | | clone_end = 3/gb = AI851062/gi = 5494968/ug = Mm 21752/len = 400/STRA = for | | | | | | | |
| 13 | 94071_at | Cluster Incl AI847904: UI-M-AI1-afl-a-09-0-UI s1 Mus musculus cDNA, 3 end/clone = UI-M-AI1-afl-a-09-0-UI/clone_end = 3/ | P | 266.6 | P | 241.5 | P | 50.9 | A |
| | | gb = AI847904/gi = 5491810/ug = Mm.22114/len = 350/STRA = for | | | | | | | |
| 13 | 94882_i_at | Cluster Incl AI850000: UI-M-BG0-aib-e-07-0-UI s1 Mus musculus cDNA, 3 end/clone = UI-M-BG0-aib-e-07-0-UI/ | P | 872.4 | P | 822.9 | P | 621.9 | P |
| | | clone_end = 3/gb = AI850000/gi = 5493906/ug = Mm 23693/len = 409/STRA = for | | | | | | | |
| 13 | 96081_at | Cluster Incl X60980.Mus musculus TK gene encoding thymidine kinase, exon 1 and 2, partial/cds = (0,97)/gb = X60980/ | P | 390.6 | P | 242.5 | P | −114.6 | A |
| | | gi = 54809/ug = Mm.2661/len = 98/STRA = for | | | | | | | |
| 13 | 96698_at | Cluster Incl AI835520: UI-M-AI0-aam-d-11-0-UI s1 Mus musculus cDNA, 3 end/clone = UI-M-AI0-aam-d-11-0-UI/ | P | 504.2 | P | 566.2 | P | 211.8 | P |
| | | clone_end = 3/gb = AI835520/gi = 5469733/ug = Mm.27558/len = 421/STRA = for | | | | | | | |
| 13 | 97295_at | Cluster Incl AW122331: UI-M-BH2 2-aos-f-09-0-UI.s1 Mus musculus cDNA, 3 end/clone = UI-M-BH2.2-aos-f-09-0-UI/ | P | 407 | P | 502.3 | P | 235.4 | A |
| | | clone_end = 3/gb = AW122331/gi = 6097816/ug = Mm 28038/len = 409/STRA = for | | | | | | | |
| 13 | 97884_at | Cluster Incl AI844175 UI-M-AL1-ahj-h-09-0-UI s1 Mus musculus cDNA, 3 end/clone = UI-M-AL1-ahj-h-09-0-UI/ | P | 195.5 | P | 409.5 | P | 76.9 | A |
| | | clone_end = 3/gb = AI844175/gi = 5488081/ug = Mm 28378/len = 319/STRA = for | | | | | | | |
| 13 | 98887_at | Cluster Incl AW125607: UI-M-BH2.2-aqj-f-06-0-UI.s1 Mus musculus cDNA, 3 end/clone = UI-M-BH2.2-aqj-f-06-0-UI/ | P | 502.4 | P | 489.5 | P | 307.3 | P |
| | | clone_end = 3/gb = AW125607/gi = 6101137/ug = Mm.28538/len = 461/STRA = for | | | | | | | |
| 13 | 94449_at | Cluster Incl AI854522: UI-M-BH0-ajj-d-05-0-UI.s1 Mus musculus cDNA, 3 end/clone = UI-M-BH0-ajj-d-05-0-UI/clone_end = 3/ | P | 527.3 | P | 528.5 | P | 247.8 | A |
| | | gb = AI854522/gi = 5498428/ug = Mm 28785/len = 358/STRA = for | | | | | | | |
| 13 | 94453_at | Cluster Incl AI852571 UI-M-BH0-aiw-h-10-0-UI s1 Mus musculus cDNA, 3 end/clone = UI-M-BH0-aiw-h-10-0-UI/ | P | 253.5 | P | 293.1 | P | 163.1 | M |
| | | clone_end = 3/gb = AI852571/gi = 5496477/ug = Mm.28794/len = 293/STRA = for | | | | | | | |
| 13 | 96262_at | Cluster Incl AI836812 UI-M-AJ0-abd-b-04-0-UI.s1 Mus musculus cDNA, 3 end/clone = UI-M-AJ0-abd-b-04-0-UI/ | P | 495.6 | P | 480.4 | P | 332.9 | P |
| | | clone_end = 3/gb = AI836812/gi = 5471025/ug = Mm 29829/len = 289/STRA = for | | | | | | | |
| 13 | 96319_at | Cluster Incl AW061324 UI-M-BH1-anw-e-09-0-UI s1 Mus musculus cDNA, 3 end/clone = UI-M-BH1-anw-e-09-0-UI/ | P | 763.3 | P | 932.3 | P | 498.2 | A |
| | | clone_end = 3/gb = AW061324/gi = 6009075/ug = Mm 29931/len = 401/STRA = for | | | | | | | |
| 13 | 97516_at | Cluster Incl U92793 Mus musculus alpha glucosidase II alpha subunit mRNA, complete cds/cds = (11,2911)/gb = U92793/ | P | 282.5 | P | 354.5 | P | 168.3 | A |
| | | gi = 2104688/ug = Mm 3196/len = 3859/STRA = for | | | | | | | |
| 13 | 98076_at | Cluster Incl AI835644 UI-M-AJ0-aau-c-02-0-UI s1 Mus musculus cDNA, 3 end/clone = UI-M-AJ0-aau-c-02-0-UI/ | P | 737.9 | P | 709.9 | P | 464 | A |
| | | clone_end = 3/gb = AI835644/gi = 5469857/ug = Mm 34536/len = 372/STRA = for | | | | | | | |
| 13 | 98617_at | Cluster Incl AF071186 Mus musculus WW domain binding protein 11 mRNA, complete cds/cds = (7,1176)/gb = AF071186/ | P | 535.8 | P | 624.7 | A | 286.2 | P |
| | | gi = 3550081/ug = Mm 3855/len = 1864/STRA = for | | | | | | | |
| 13 | 99123_s_at | Cluster Incl AW061280 UI-M-BH1-anw-a-07-0-UI s1 Mus musculus cDNA, 3 end/clone = UI-M-BH1-anw-a-07-0-UI/ | P | 125.5 | P | 57 | P | −46 | A |
| | | clone_end = 3/gb = AW061280/gi = 6009031/ug = Mm 4050/len = 155/STRA = for | | | | | | | |
| 13 | 100156_at | Cluster Incl D26090 Mouse mRNA for mCDC46 protein, complete cds/cds = (84,2285)/gb = D26090/gi = 940403/ | P | 631.3 | P | 607.2 | P | 196 | A |
| | | ug = Mm 5048/len = 2649/STRA = for | | | | | | | |
| 13 | 100559_at | Cluster Incl AI853344.UI-M-BH0-air-b-10-0-UI.s1 Mus musculus cDNA, 3 end/clone = UI-M-BH0-air-b-10-0-UI/ | P | 275.8 | P | 235.4 | P | 58.1 | P |
| | | clone_end = 3/gb = AI853344/gi = 5497250/ug = Mm 5624/len = 336/STRA = for | | | | | | | |
| 13 | 100564_at | Cluster Incl AF068199 D-dopachrome tautomerase/cds = (0,356)/gb = AF068199/gi = 3452318/ug = Mm 5731/len = 357/ | P | 265.5 | P | 188.4 | P | 100.6 | A |
| | | STRA = for | | | | | | | |
| 13 | 102354_at | Cluster Incl AI049398 ub33c08 r1 Mus musculus cDNA, 5 end/clone = IMAGE-1379534/clone_end = 5/gb = AI049398/ | P | 332.7 | P | 175.2 | P | 185.3 | A |
| | | gi = 3297685/ug = Mm 11434/len = 420/STRA = for | | | | | | | |
| 13 | 102383_at | Cluster Incl AW048977.UI-M-BH1-amo-h-10-0-UI s1 Mus musculus cDNA, 3 end/clone = UI-M-BH1-amo-h-10-0-UI/ | P | 214.8 | P | 147.6 | P | 106.7 | A |
| | | clone_end = 3/gb = AW048977/gi = 5909506/ug = Mm 12180/len = 470/STRA = for | | | | | | | |
| 13 | 103799_at | Cluster Incl AF073881 Mus musculus myotubulann homologous protein 3 mRNA, partial cds/cds = (0,365)/gb = AF073881/ | P | 71.6 | P | 78 | P | 54.2 | M |
| | | gi = 3916225/ug = Mm 20844/len = 940/STRA = for | | | | | | | |
| 13 | 103889_at | Cluster Incl AI834833.UI-M-AM0-ads-g-05-0-UI s1 Mus musculus cDNA, 3 end/clone = UI-M-AM0-ads-g-05-0-UI/ | P | 362.1 | P | 336.2 | P | 199.6 | P |
| | | clone_end = 3/gb = AI834833/gi = 5469046/ug = Mm 21286/len = 407/STRA = for | | | | | | | |
| 13 | 95527_at | Cluster Incl AJ132771 Mus musculus mRNA for chromatin assembly factor-1 p150 subunit/cds = (0,2735)/gb = AJ132771/ | P | 17.4 | P | 69.7 | P | −95.4 | P |
| | | gi = 5852157/ug = Mm 27609/len = 2802/STRA = for | | | | | | | |
| 13 | 95541_at | Cluster Incl AW125506: UI-M-BH2.2-aqm-e-08-0-UI.s1 Mus musculus cDNA, 3 end/clone = UI-M-BH2.2-aqm-e-08-0-UI/ | P | 104.1 | P | 112.9 | P | −24.6 | A |
| | | clone_end = 3/gb = AW125506/gi = 6101036/ug = Mm 27673/len = 434/STRA = rev | | | | | | | |
| 13 | 95634_at | Cluster Incl AI848107 UI-M-AP1-agk-e-09-0-UI s1 Mus musculus cDNA, 3 end/clone = UI-M-AP1-agk-e-09-0-UI/ | P | 159.4 | P | 158.4 | P | 82.7 | P |
| | | clone_end = 3/gb = AI848107/gi = 5492013/ug = Mm 28094/len = 475/STRA = for | | | | | | | |
| 13 | 96753_at | Cluster Incl Y11905 M. musculus BCL7C mRNA/cds = (57,710)/gb = Y11905/gi = 1946223/ug = Mm 2898/len = 781/STRA = for | P | 137.4 | P | 205.4 | P | 98.6 | P |
| 13 | 96763_at | Cluster Incl AI839995: UI-M-AH0-acs-f-11-0-UI s1 Mus musculus cDNA, 3 end/clone = UI-M-AH0-acs-f-11-0-UI/ | P | 110 | P | 154.1 | P | 82.5 | A |
| | | clone_end = 3/gb = AI839995/gi = 5474208/ug = Mm 29007/len = 357/STRA = for | | | | | | | |
| 13 | 98437_at | Cluster Incl U63720 Mus musculus CPP32 apoptotic protease mRNA, partial cds/cds = (0,662)/gb = U63720/gi = 4097524/ | P | 934 | P | 620.9 | P | 431.8 | A |
| | | ug = Mm 34405/len = 1609/STRA = for | | | | | | | |
| 13 | 100513_at | Cluster Incl AF075461 Mus musculus ADP-nbosylation factor-directed GTPase activating protein isoform a (Shag1) mRNA, | P | −127.9 | P | −247.5 | P | −422.1 | P |
| | | complete cds/cds = (49,3492)/gb = AF075461/gi = 4063613/ug = Mm 5237/len = 4370/STRA = rev | | | | | | | |
| 13 | 100989_at | Cluster Incl AJ001373 Mus musculus bodenin gene/cds = (173,775)/gb = AJ001373/gi = 2647955/ug = Mm 6818/len = 954/ | P | 135.9 | P | 72.8 | P | 0.8 | A |
| | | STRA = for | | | | | | | |
| 13 | 101854_r_at | Cluster Incl AI844178 UI-M-AL1-ahk-a-02-0-UI s1 Mus musculus cDNA, 3 end/clone = UI-M-AL1-ahl-a-02-0-UI/ | P | 892.5 | P | 1121.9 | P | 764.5 | A |
| | | clone_end = 3/gb = AI844178/gi = 5488084/ug = Mm 866/len = 298/STRA = for | | | | | | | |
| 13 | 103711_at | Cluster Incl AI115399: uh85b06.r1 Mus musculus cDNA, 5 end/clone = IMAGE-1764467/clone_end = 5/gb = AI115399/ | P | 146.5 | P | 92.1 | P | 70.7 | M |
| | | gi = 3515723/ug = Mm 24100/len = 450/STRA = for | | | | | | | |
| 13 | 92758_at | Cluster Incl U09268 Mus musculus domesticus 129 tyrosine-threonine dual specificity phosphatase PAC-1 gene, complete | P | 555.2 | P | 679.3 | P | 479.8 | A |
| | | cds/cds = (0,539)/gb = U09268/gi = 505624/ug = Mm 4729/len = 540/STRA = for | | | | | | | |
| 13 | 93698_at | Cluster Incl AB006329 Mus musculus mSox13 mRNA, complete cds/cds = (48,1835)/gb = AB006329/gi = 3077735/ | P | 1008.9 | P | 1172.9 | P | 920.3 | M |
| | | ug = Mm 8575/len = 3183/STRA = rev | | | | | | | |
| 13 | 95374_f_at | Cluster Incl AI413993 ma76g11.x1 Mus musculus cDNA, 3 end/clone = IMAGE-316676/clone_end = 3/gb = AI413993/ | P | 668.4 | P | 772.3 | P | 128.3 | M |
| | | gi = 4257497/ug = Mm 22881/len = 308/STRA = for | | | | | | | |
| 13 | AFFX- | L09192 Mus musculus pyruvate carboxylase mRNA, complete cds (_5, _MA, MB, _3 | P | 472.4 | P | 490.2 | P | 154.5 | A |
| | PyruCarbMur/ | represent transcript regions 5 prime, | | | | | | | |
| | L09192_3_at | MiddleA, MiddleB, and 3 prime respectively) | | | | | | | |
| 13 | 92879_at | Cluster Incl U42383 Fibroblast growth factor inducible 13/cds = (9,1637)/gb = U42383/gi = 3318654/ug = Mm 14501/ | P | 510.3 | P | 409 | P | 301.8 | A |
| | | len = 2069/STRA = for | | | | | | | |
| 13 | 93803_at | Cluster Incl AB007139 Mus musculus Psme3 gene for PA28 gamma subunit, complete cds/cds = (119,883)/gb = AB007139/ | P | 320.2 | P | 278 | P | 169.1 | A |
| | | gi = 3219287/ug = Mm 2141/len = 2555/STRA = for | | | | | | | |
| 13 | 94015_at | Cluster Incl AW060447 UI-M-BH1-anj-f-02-0-UI s1 Mus musculus cDNA, 3 end/clone = UI-M-BH1-anj-f-02-0-UI/ | P | 278.7 | P | 335 | M | 177.1 | A |
| | | clone_end = 3/gb = AW060447/gi = 6006198/ug = Mm 2184/len = 460/STRA = for | | | | | | | |
| 13 | 97271_at | Cluster Incl AI845593 UI-M-AQ1-adx-d-04-0-UI.s1 Mus musculus cDNA, 3 end/clone = UI-M-AQ1-adx-d-04-0-UI/ | P | 295.9 | P | 353.8 | P | 97.6 | A |
| | | clone_end = 3/gb = AI845593/gi = 5489499/ug = Mm.27922/len = 390/STRA = for | | | | | | | |
| 13 | 98069_s_at | Cluster Incl AI854467 UI-M-BH0-ait-g-02-0-UI s1 Mus musculus cDNA, 3 end/clone = UI-M-BH0-ait-g-02-0-UI/clone_end = 3/ | P | 250.4 | P | 262.2 | P | 67.7 | A |
| | | gb = AI854467/gi = 5498373/ug = Mm 34456/len = 390/STRA = for | | | | | | | |
| 13 | 101485_at | Cluster Incl AW124859 UI-M-BH2.1-apu-a-02-0-UI.s1 Mus musculus cDNA, 3 end/clone = UI-M-BH2.1-apu-a-02-0-UI/ | P | 346.1 | P | 210.1 | M | 89.6 | M |
| | | clone_end = 3/gb = AW124859/gi = 6100389/ug = Mm 7861/len = 372/STRA = for | | | | | | | |
| 13 | 101967_at | Cluster Incl D50646: Mouse mRNA for SDF2, complete cds/cds = (28,663)/gb = D50646/gi = 1741869/ug = Mm.935/len = 1012/ | P | 482.2 | P | 567.9 | P | 465.9 | A |
| | | STRA = for | | | | | | | |
| 13 | 103884_at | Cluster Incl AI850335: UI-M-BG1-alf-g-11-0-UI.s1 Mus musculus cDNA, 3 end/clone = UI-M-BG1-aif-g-11-0-UI/ | P | −115.4 | P | −249.4 | P | −280.6 | A |
| | | clone_end = 3/gb = AI850335/gi = 5494241/ug = Mm 21241/len = 475/STRA = for | | | | | | | |
| 13 | 104583_at | Cluster Incl AI227492 uf10c07 y1 Mus musculus cDNA, 5 end/clone = IMAGE-1510956/clone_end = 5/gb = AI227492/ | P | 205.4 | P | 171.2 | A | 80.2 | P |
| | | gi = 3810545/ug = Mm 23975/len = 655/STRA = for | | | | | | | |
| 13 | 95612_at | Cluster Incl AA667128 vq89a03.r1 Mus musculus cDNA, 5 end/clone = IMAGE-1109452/clone_end = 5/gb = AA667128/ | P | 465.4 | P | 309.7 | P | 247.2 | M |
| | | gi = 2625829/ug = Mm.27997/len = 108/STRA = for | | | | | | | |
| 13 | 96125_at | Cluster Incl AF110520: Mus musculus major histocompatibllity complex region NG27, NG28, RPS28, NADH | A | 498.9 | P | 601.5 | P | 414.1 | P |
| | | oxidoreductase, NG29, KIFC1, Fas-binding protein, BING1, tapasin, RalGDS-like, KE2, BING4, beta 1,3-galactosyl | | | | | | | |
| | | transferase, and RPS18 genes, complete cds, S/STRA = for | | | | | | | |
| 13 | 96234_at | Clusler Incl AI849311, UI-M-AJ1-aha-c-04-0-UI.s1 Mus musculus cDNA, 3 end/clone = UI-M-AJ1-aha-c-04-0-UI/ | P | 179.5 | P | 118.7 | P | 3.4 | A |
| | | clone_end = 3/gb = AI849311/gi = 5493217/ug = Mm 28790/len = 471/STRA = for | | | | | | | |
| 13 | 100004_at | Cluster Incl AW120890: UI-M-BH2.3-aob-a-12-0-UI s1 Mus musculus cDNA, 3 end/clone = UI-M-BH2 3-aob-a-12-0-UI/ | P | 330.7 | P | 370.6 | P | 307.1 | M |
| | | clone_end = 3/gb = AW120890/gi = 6096223/ug = Mm 45194/len = 364/STRA = for | | | | | | | |
| 13 | 104259_at | Cluster Incl X99641 M musculus mRNA for HP1 alpha protein/cds = (63,638)/gb = X99641/gi = 1480107/ug = Mm 28408/ | P | 299.8 | P | 327.2 | P | 255.5 | A |
| | | len = 865/STRA = for | | | | | | | |
| 13 | 92332_at | Cluster Incl M80540 Distal-less homeobox 2/cds = (300,1298)/gb = M80540/gi = 201920/ug = Mm 3896/len = 2408/STRA = for | P | 62.2 | P | 79.2 | P | −13.1 | A |
| 13 | 98355_at | Cluster Incl AA544993-vk42e09 r1 Mus musculus cDNA, 5 end/clone = IMAGE-949288/clone_end = 5/gb = AA544993/ | A | 244.8 | A | 402.1 | P | 195.4 | P |
| | | gi = 2306067/ug = Mm 4393/len = 297/STRA = for | | | | | | | |
| 13 | 99807_r_at | Cluster Incl X97720 Friend virus susceptibility 1/cds = (0,1322)/gb = X97720/gi = 1515301/ug = Mm 57220/len = 1323 | P | 182.5 | P | 233.1 | P | 80.7 | A |
| | | STRA = for | | | | | | | |
| 13 | 100684_at | Cluster Incl U92794-Proteln kinase C substrate 80K-H/cds = (114,1679)/gb = U92794/gi = 2104690/ug = Mm 14244/len = 2030/ | A | 412.8 | P | 497.2 | P | 398.7 | P |
| | | STRA = for | | | | | | | |
| 13 | 101508_at | Cluster Incl U17961: Mus musculus p62 mRNA, complete cds/cds = (111,1442)/gb = U17961/gi = 602250/ug = Mm 8256/ | A | 234.4 | P | 210.6 | A | 170 | A |
| | | len = 1765/STRA = for | | | | | | | |
| 13 | 103625_at | Cluster Incl AA797556 vw25a08 r1 Mus musculus cDNA, 5 end/clone = IMAGE-1244822/clone_end = 5/gb = AA797556/ | P | 327.1 | P | 302 | P | 241.3 | A |
| | | gi = 2860511/ug = Mm 20279/len = 427/STRA = for | | | | | | | |
| 13 | 104301_at | Cluster Incl AI852414 UI-M-BH0-aip-e-01-0-UI s1 Mus musculus cDNA, 3 end/clone = UI-M-BH0-aip-e-01-0-UI/ | P | 264.2 | P | 233.1 | A | 25.5 | P |
| | | clone_end = 3/gb = AI852414/gi = 5496360/ug = Mm.22351/len = 425/STRA = for | | | | | | | |
| 13 | 94356_at | Cluster Incl AI593074 vv10f08.x1 Mus musculus cDNA, 3 end/clone = IMAGE-1211267/clone_end = 3/gb = AI593074/ | P | 71 | P | 69.2 | A | 40.1 | A |
| | | gi = 4602122/ug = Mm 25231/len = 604/STRA = rev | | | | | | | |
| 13 | 96189_at | Cluster Incl AW046184 UI-M-BH1-akw-d-10-0-UI s1 Mus musculus cDNA, 3 end/clone = UI-M-BH1-akw-d-10-0-UI/ | A | 53 | P | 23.6 | P | 3.6 | A |
| | | clone_end = 3/gb = AW046184/gi = 5906713/ug = Mm 28502/len = 416/STRA = for | | | | | | | |
| 13 | 99023_at | Cluster Incl U57747 Mus musculus platelet-activating factor acetylhydrolase isoform Ib beta subunit mRNA, complete cds/ | P | 340.1 | P | 295.2 | P | 192 | A |
| | | cds = (106,795)/gb = U57747/gi = 1373362/ug = Mm 3877/len = 944/STRA = for | | | | | | | |
| 13 | 100461_at | Cluster Incl D85818 Mouse mRNA for RNA polymerase II subuunit RPB14, complete cds/cds = (16,369)/gb = D85818/ | P | 634.8 | P | 701.4 | P | 263.5 | A |
| | | gi = 2081615/ug = Mm 4896/len = 531/STRA = for | | | | | | | |
| 13 | 100908_at | Cluster Incl M36033 Protein tyrosine phosphatase, receptor type, A/cds = (25,2514)/gb = M36033/gi = 198876/ug = Mm 587/ | P | 153.1 | P | 113.7 | A | 96 | A |
| | | len = 3070/STRA = for | | | | | | | |
| 13 | 101856_at | Cluster Incl AI836771.UI-M-AJ0-abc-d-02-0-UI.s1 Mus musculus cDNA, 3 end/clone = UI-M-AJ0-abc-d-02-0-UI/ | P | 174 | P | 259.7 | A | 172.3 | A |
| | | clone_end = 3/gb = AI836771/gi = 5470964/ug = Mm 86933/len = 200/STRA = for | | | | | | | |
| 13 | 95765_at | Cluster Incl U48971 Mus musculus class III alcohol dehydrogenase pseudogene/cds = (0,1124)/gb = U48971/gi = 1399469/ | P | 565.5 | P | 431.5 | P | 229.6 | A |
| | | ug = Mm 87751/len = 1125/STRA = for | | | | | | | |
| 13 | 97249_at | Cluster Incl AW124204 UI-M-BH2.1-aph-b-11-0-UI s1 Mus musculus cDNA, 3 end/clone = UI-M-BH2 1-aph-b-11-0-UI/ | P | 260.3 | P | 358.8 | P | 227.5 | A |
| | | clone_end = 3/gb = AW124204/gi = 6099734/ug = Mm 27853/len = 382/STRA = for | | | | | | | |
| 13 | 95759_at | Cluster Incl AW123746: UI-M-BH2 1-apl-h-09-0-UI s1 Mus musculus cDNA, 3 end/clone = UI-M-BH2 1-apl-h-09-0-UI/ | P | 119.7 | P | 105.4 | A | −39.7 | A |
| | | clone_end = 3/gb = AW123746/gi = 6099276/ug = Mm 298/len = 462/STRA-for | | | | | | | |
| 13 | 99139_at | Cluster Incl AW123783 UI-M-BH2 1-apm-d-01-0-UI s1 Mus musculus cDNA, 3 end/clone = UI-M-BH2.1-apm-d-01-0-UI/ | P | 265.2 | P | 361.1 | P | 146.1 | A |
| | | clone_end = 3/gb = AW123783/gi = 6099313/ug = Mm 41374/len = 404/STRA = for | | | | | | | |
| 13 | 101054_at | Cluster Incl X00496 Mouse la-associated invariant chain (II) mRNA fragment/cds = (0,509)/gb = X00496/gi = 53102/ | P | 399 | P | 129.6 | A | 24 | A |
| | | ug = Mm.7043/len = 994/STRA = for | | | | | | | |
| 13 | 101074_at | Cluster Incl D89063: Mus musculus mRNA for oligosaccharyltransferase, complete cds/cds = (143,1468)/gb = D89063/ | P | 155.8 | A | 202.6 | P | 88.7 | A |
| | | gi = 2687875/ug = Mm 7236/len = 1750/STRA = for | | | | | | | |
| 13 | 103871_at | Cluster Incl AW123729 UI-M-BH2 1-apl-f-11-0-UI s1 Mus musculus cDNA, 3 end/clone = UI-M-BH2 1-apl-f-11-0-UI/ | P | 17.9 | P | 53.2 | A | −165.8 | A |
| | | clone_end = 3/gb = AW123729/gi = 6099259/ug = Mm 21163/len = 462/STRA = for | | | | | | | |
| 13 | 94407_at | Cluster Incl AI835481 UI-M-AQ0-aag-e-02-0-UI s1 Mus muaculus cDNA, 3 end/clone = UI-M-AQ0-aag-e-02-0-UI/ | P | 242.2 | P | 315 | A | 176.6 | A |
| | | clone_end = 3/gb = AI835481/gi = 5469694/ug = Mm 259/len = 220/STRA = for | | | | | | | |
| 13 | 96121_at | Cluster Incl AW047866 UI-M-BH1-als-f-05-0-UI s1 Mus musculus cDNA, 3 end/clone = UI-M-BH1-als-f-05-0-UI/ | P | 164.1 | A | 139.8 | A | 31.3 | A |
| | | clone_end = 3/gb = AW047866/gi = 5908395/ug = Mm 28103/len = 411/STRA = rev | | | | | | | |
| 13 | 94644_at | Cluster Incl M99492 Mouse butyrylcholinesterase mRNA, complete cds/cds = (17,1828)/gb = M99492/gi = 191579/ | A | 39.4 | P | 25.4 | P | 5 | P |
| | | ug = Mm.8073/len = 1891/STRA = for | | | | | | | |
| 13 | 93303_at | Cluster Incl U64445 Ubiquitin fusion degradation 1 —like/cds = (106,1029)/gb = u64445/gi = 1654347/ug = Mm.1830/ | P | 208.2 | P | 134.9 | P | 79.4 | A |
| | | len = 1479/STRA = for | | | | | | | |
| 13 | 97238_at | Cluster Incl AW209238: uo07g04.x1 Mus musculus cDNA, 3 end/clone = IMAGE-2598870/clone_end = 3/gb = AW209238/ | A | 78.4 | P | 49.4 | A | −3.4 | A |
| | | gi = 6515178/ug = Mm 27836/len = 568/STRA = for | | | | | | | |
| 13 | 97251_at | Cluster Incl AI842124: UI-M-AN1-afg-h-03-0-UI s1 Mus musculus cDNA, 3 end/clone = UI-M-AN1-afg-h-03-0-UI/ | P | 136.2 | M | 122.9 | A | 66 | A |
| | | clone_end = 3/gb = AI842124/gi = 5476337/ug = Mm 27863/len = 301/STRA = for | | | | | | | |
| 13 | 100526_f_at | Cluster Incl X84227 Cyritestin 1/cds = (72,2540)/gb = X64227/gi = 1666660/ug = Mm 5168/len = 2722/STRA = for | P | 102.1 | P | 166.5 | P | 60.4 | A |
| 13 | 94382_at | Cluster Incl AI851901.UI-M-BH0-aix-d-03-0-UI s1 Mus musculus cDNA, 3 end/clone = UI-M-BH0-aix-d-03-0-UI/ | A | 140.5 | P | 99.6 | A | 90.9 | A |
| | | clone_end = 3/gb = AI851901/gi = 5495807/ug = Mm 25608/len = 428/STRA = for | | | | | | | |
| 13 | 99459_f_at | Cluster Incl X70764: ELKL motif kinase/cds = (93,2417)/gb = X70764/gi = 57919/ug = Mm.4082/len = 2763/STRA = for | P | 107.2 | P | 55.6 | A | −10.1 | A |
| 13 | 100026_at | Cluster Incl U42443: Mus musculus MECA39 mRNA, complete cds/cds = (0,1160)/gb = U42443/gi = 1480454/ug = Mm.4606/ | P | 113.7 | A | 274.7 | A | 65 | A |
| | | len = 1161/STRA = for | | | | | | | |
| 13 | 93612_at | Cluster Incl D86332 Matrix metalloproteinase 15/cds = (28,2001)/gb = D86332/gi = 2687872/ug = Mm 7283/len = 3339/ | A | 98.3 | P | 122.3 | P | 31 | A |
| | | STRA = for | | | | | | | |
| 13 | 94713_at | Cluster Incl U81453 Myosin VIIa/cds = (10,6657)/gb = U81453/gi = 1778381/ug = Mm 1403/len = 7135/STRA = rev | A | 272.3 | A | 328.5 | P | 119.9 | P |
| 13 | 95514_at | Cluster Incl AI846302 UI-M-AK1-aez-g-04-0-UI s1 Mus musculus cDNA, 3 end/clone = UI-M-AK1-aez-g-04-0-UI/ | P | 223.5 | P | 317.8 | P | −158.6 | A |
| | | clone_end = 3/gb = AI846302/gi = 5490208/ug = Mm 253/len = 324/STRA = rev | | | | | | | |
| 13 | 104078_g_at | Cluster Incl AW121992 UI-M-BH2 3-aoj-f-11-0-UI.s1 Mus musculus cDNA, 3 end/clone = UI-M-BH2.3-aoj-f-11-0-UI/ | M | 73.4 | P | 113.3 | P | −79.8 | A |
| | | clone_end = 3/gb = AW121992/gi = 6097398/ug = Mm 21705/len = 477/STRA = for | | | | | | | |
| 13 | 94933_at | Cluster Incl AI853739 UI-M-BH0-ajg-a-12-0-UI.s1 Mus musculus cDNA, 3 end/clone = UI-M-BH0-ajg-a-12-0-UI/ | P | 387.5 | P | 295.2 | A | 167.9 | A |
| | | clone_end = 3/gb = AI853739/gi = 5497645/ug = Mm.26783/len = 346/STRA = for | | | | | | | |
| 13 | 98493_at | Cluster Incl AA867026 vx22e02 r1 Mus musculus cDNA, 5 end/clone = IMAGE-1265210/clone_end = 5/gb = AA867026/ | P | 25.2 | P | 29.2 | M | −69.7 | A |
| | | gi = 2962471/ug = Mm 35606/len = 269/STRA = for | | | | | | | |
| 13 | 98994_at | Cluster Incl AF081499 Mus musculus type IIb Na/phosphate-cotransporter (Npt2b) mRNA, complete cds/cds = (44,2137)/ | A | 120.8 | P | 42.7 | A | −50.9 | A |
| | | gb = AF081499/gi = 3929576/ug = Mm 3786/len = 4039/STRA = for | | | | | | | |
| 13 | 99548_at | Cluster Incl AF033034 Mus musculus aldehyde dehydrogenase 3 (aldh3) gene, partial cds/cds = (45,155)/gb = AF033034/ | A | −311 | P | −232 | A | −626.5 | M |
| | | gi = 2645710/ug = Mm 4257/len = 156/STRA = for | | | | | | | |
| 13 | 99961_s_at | Cluster Incl L37092 Mus musculus cyclin-dependent kinase homologue (p130PITSL) mRNA, complete cds/cds = (661,3012)/ | A | 80.6 | P | −6.7 | A | −73.1 | M |
| | | gb = L37092/gi = 561745/ug = Mm 4414/len = 3161/STRA = for | | | | | | | |
| 13 | 96976_f_at | Cluster Incl X16217 Mouse T11 class I MHC gene (exon 5)/cds = (0,88)/gb = X16217/gi = 54221/ug = Mm 88813/len = 113/ | P | 254.1 | P | 140.7 | A | 14.3 | M |
| | | STRA = for | | | | | | | |
| 14 | 92571_at | Cluster Incl D85904 Mouse mRNA for apg-2, complete cds/cds = (15,2540)/gb = D85904/gi = 1389636/ug = Mm 1032/ | P | 481.4 | P | 309 | P | 466.5 | P |
| | | len = 2582/STRA = for | | | | | | | |
| 14 | 92580_at | Cluster Incl U39473 Mus musculus histidyl-tRNA synthetase mRNA, complete cds/cds = (85,1614)/gb = U39473/ | P | 390.1 | P | 213.9 | P | 416.6 | P |
| | | gi = 1209682/ug = Mm 10528/len = 1629/STRA = for | | | | | | | |
| 14 | 92598_at | Cluster Incl AI843029.UI-M-AK1-aeq-e-08-0-UI s1 Mus musculus cDNA, 3 end/clone = UI-M-AK1-aeq-e-08-0-UI/ | P | 1160.5 | P | 833.6 | P | 1229.4 | P |
| | | clone_end = 3/gb = AI843029/gi = 5477242/ug = Mm 10727/len = 298/STRA = for | | | | | | | |
| 14 | 93059_at | Cluster Incl AI854841 UI-M-BH0-akb-b-10-0-UI s1 Mus musculus cDNA, 3 end/clone = UI-M-BH0-akd-b-10-0-UI/ | P | 416.5 | P | 222.3 | P | 346.5 | P |
| | | clone_end = 3/gb = AI854841/gi = 5498747/ug = Mm 155/len = 396/STRA = for | | | | | | | |
| 14 | 93256_at | Cluster Incl AI853728 UI-M-BH0-ajq-e-09-0-UI s1 Mus musculus cDNA, 3 end/clone = UI-M.BH0-ajq-e-09-0-UI/ | P | 1055.6 | P | 921.1 | P | 1013.7 | P |
| | | clone_end = 3/gb = AI853728/gi = 5497599/ug = Mm 17035/len = 482/STRA = for | | | | | | | |
| 14 | 93312_at | Cluster Incl AW124623 UI-M-BH2 1-apw-b-04-0-UI s1 Mus musculus cDNA, 3 end/clone = UI-M-BH2.1-apw-b-4-0-UI/ | P | 856.4 | P | 630.2 | P | 807.3 | P |
| | | clone_end = 3/gb = AW124623/gi = 6100153/ug = Mm 18485/len = 422/STRA = for | | | | | | | |
| 14 | 93319_at | Cluster Incl U20238 Mus musculus GTPase-activating protein GAPIII mRNA, complete cds/cds = (125,2629)/gb-U20238/ | P | 480.7 | P | 424.1 | P | 452.4 | P |
| | | gi = 972943/ug = Mm.18517/len = 4107/STRA = for | | | | | | | |
| 14 | 93580_at | Cluster Incl AI837010 UI-M-AJ0-aaz-h-06-0-UI s1 Mus musculus cDNA, 3 end/clone = UI-M-AJ0-aaz-h-06-0-UI/ | P | 1171.1 | P | 828.3 | P | 954.8 | P |
| | | clone_end = 3/gb = AI837010/gi = 5471223/ug = Mm 20543/len = 472/STRA = for | | | | | | | |
| 14 | 94018_at | Cluster Incl AW120725 UI-M-BH2 3-any-a-12-0-UI s1 Mus musculus cDNA, 3 end/clone = UI-M-BH2 3-any-a-12-0-UI/ | P | 1600.3 | P | 1060.6 | P | 2023.2 | P |
| | | clone_end = 3/gb = AW120725/gi = 6096058/ug = Mm 21846/len = 227/STRA = for | | | | | | | |
| 14 | 94245_at | Cluster Incl AW125865 UI-M-BH2 2-aqk-e-12-0-UI s1 Mus musculus cDNA, 3 end/clone = UI-M-BH2 2-aqk-e-12-0-UI/ | P | 272.6 | P | 124.6 | P | 214.3 | P |
| | | clone_end = 3/gb = AW125865/gi = 6101395/ug = Mm 22362/len = 178/STRA = for | | | | | | | |
| 14 | 94834_at | Cluster Incl U06119 Cathepsin H/cds = (47,1048)/gb = U06119/gi = 454100/ug = Mm 2277/len = 1381/STRA = for | P | 757.7 | P | 591.9 | P | 702.6 | P |
| 14 | 94893_at | Cluster Incl AI840411 UI-M-AH0-acv-h-03-0-UI.s1 Mus musculus cDNA, 3 end/clone = UI-M-AH0-acv-h-03-0-UI/ | P | 143.1 | P | 15.9 | P | 145.4 | P |
| | | clone_end = 3/gb = AI840411/gi = 5474624/ug = Mm.23962/len = 435/STRA = for | | | | | | | |
| 14 | 95425_at | Cluster Incl U21489 Acetyl coenzyme A dehydrogenase, long-chain/cds = (0,1292)/gb = U21489/gi = 726094/ug = Mm.2445/ | P | 641.3 | P | 349.9 | P | 498.8 | P |
| | | len = 1434/STRA = for | | | | | | | |
| 14 | 96038_at | Cluster Incl AI840339 UI-M-AJ0-abd-g-01-0-UI S2 Mus musculus cDNA, 3 end/clone = UI-M-AJ0-abd-g-01-0-UI/ | P | 1082.3 | P | 355 | P | 983.3 | P |
| | | clone_end = 3/gb = AI840339/gi = 5474552/ug = Mm 2587/len = 497/STRA = for | | | | | | | |
| 14 | 96079_at | Cluster Incl AI853881 UI-M-BH0-aju-a-01-0-UI s1 Mus musculus cDNA, 3 end/clone = UI-M-BH0-aju-a-01-0-UI/ | P | 464 | P | 313.4 | P | 410.8 | P |
| | | clone_end = 3/gb = AI853881/gi = 5497787/ug = Mm 26598/len = 313/STRA = for | | | | | | | |
| 14 | 96088_at | Cluster Incl AB033921 Mus musculus mRNA for Ndr1 related protein Ndr2, complete cds/cds = (35,1150)/gb = AB033921/ | P | 442 | P | 353.9 | P | 553.9 | P |
| | | gi = 6141565/ug = Mm 26722/len = 2010/STRA = for | | | | | | | |
| 14 | 96680_at | Cluster Incl AI835630 UI-M-AI0-aap-h-02-0-UI a1 Mus musculus cDNA, 3 end/clone = UI-M-AI0-aap-h-02-0-UI/ | P | 275.8 | P | 97.8 | P | 206 | P |
| | | clone_end = 3/gb = AI835630/gi = 5469843/ug = Mm 27432/len = 327/STRA = for | | | | | | | |
| 14 | 97217_at | Cluster Incl AI842095 UI-M-AN1-afg-e-02-0-UI.s1 Mus musculus cDNA, 3 end/clone = UI-M-AN1-afg-e-02-0-UI/ | P | 1529.6 | P | 1305.6 | P | 1582.9 | P |
| | | clone_end = 3/gb = AI842095/gi = 5476308/ug = Mm 27733/len = 418/STRA = for | | | | | | | |
| 14 | 97241_at | Cluster Incl AI787713 ul21fo4.y1 Mus musculus cDNA, 5 end/clone = IMAGE-2088223/clone_end = 5/gb = AI787713/ | P | 471.8 | P | 445.3 | P | 478.1 | P |
| | | gi = 5335420/ug = Mm 27839/len = 528/STRA = for | | | | | | | |
| 14 | 97802_at | Cluster Incl AI850195: UI-M-BG1-aie-a-03-0-UI.s1 Mus musculus cDNA, 3 end/clone = UI-M-BG1-aie-a-03-0-UI/ | P | 307.3 | P | 236.2 | P | 287.5 | P |
| | | clone_end = 3/gb = AI850195/gi = 5494101/ug = Mm28128/len = 531/STRA = for | | | | | | | |
| 14 | 97868_at | Cluster Incl AW060270.UI-M-BH1-amv-g-11-0-UI s1 Mus musculus cDNA, 3 end/clone = UI-M-BH1-amv-g-11-0-UI/ | P | 707.9 | P | 672.7 | P | 834.9 | P |
| | | clone_end = 3/gb = AW060270/gi = 6008061/ug = Mm 28330/len = 515/STRA = for | | | | | | | |
| 14 | 98880_at | Cluster Incl AW124955-UI-M-BH2 1-apv-a-10-0-UI s1 Mus musculus cDNA, 3 end/clone = UI-M-BH2 1-apv-a-10-0-UI/ | P | 679 | P | 474 | P | 670.7 | P |
| | | clone_end = 3/gb = AW124955/gi = 6100485/ug = Mm28524/len = 316/STRA = for | | | | | | | |
| 14 | 98931_at | Cluster Incl AW123589: UI-M-BH2 1-api-b-04-0-UI s1 Mus musculus cDNA, 3 end/clone = UI-M-BH2 1-api-b-04-0-UI/ | P | 390.2 | P | 177.2 | P | 309.8 | P |
| | | clone_end = 3/gb = AW123589/gi = 6099119/ug = Mm 26670/len = 421/STRA = for | | | | | | | |
| 14 | 98945_at | Cluster Incl AW125190.UI-M-BH2 1-apz-h-07-0-UI s1 Mus musculus cDNA, 3 end/clone = UI-M-BH2 1-apz-h-07-0-UI/ | P | 416.8 | P | 321.9 | P | 424 | P |
| | | clone_end = 3/gb = AW125190/gi = 6100720/ug = Mm 28714/len = 396/STRA = for | | | | | | | |
| 14 | 98950_at | Cluster Incl AB017616 Mus musculus YGR163W mRNA homologue, complete cds/cds = (24,1220)/gb = AB017616/ | P | 1074 | P | 652.2 | P | 919.7 | P |
| | | gi = 4519622/ug = Mm 28746/len = 2507/STRA = for | | | | | | | |
| 14 | 94469_at | Cluster Incl AW120950: UI-M-BH2.3-aod-b-02-0-UI.s1 Mus musculus cDNA, 3 end/clone = UI-M-BH2.3-aod-b-02-0-UI/ | P | 819.6 | P | 558.9 | P | 872.4 | P |
| | | clone_end = 3/gb = AW120950/gi = 6096283/ug = Mm.28848/len = 336/STRA = for | | | | | | | |
| 14 | 94470_i_at | Cluster Incl AW045974: UI-M-BH1-alc-a-08-0-UI s1 Mus musculus cDNA, 3 end/clone = UI-M-BH1-alc-a-08-0-UI/ | P | 335.7 | P | 225.1 | P | 303.7 | P |
| | | clone_end = 3/gb = AW045974/gi = 5906503/ug = Mm 28851/len = 338/STRA = for | | | | | | | |
| 14 | 94478_at | Cluster Incl AI841377 UI-M-AM0-adv-g-04-0-UI s1 Mus musculus cDNA, 3 end/clone = UI-M-AM0-adv-g-04-0-UI/ | P | 1336.4 | P | 649.3 | P | 972.7 | P |
| | | clone_end = 3/gb = AI841377/gi = 5475590/ug = Mm.28872/len = 527/STRA = for | | | | | | | |
| 14 | 95103_at | Cluster Incl AI839824 UI-M-AN0-acq-c-09-0-UI s1 Mus musculus cDNA, 3 end/clone = UI-M-AN0-acq-c-09-0-UI/ | P | 651.2 | P | 557. 8 | P | 608.9 | P |
| | | clone_end = 3/gb = AI839824/gi = 5474002/ug = Mm 29348/len = 481/STRA = for | | | | | | | |
| 14 | 95695_at | Cluster Incl AB017112 Mus musculus mCAC gene, complete cds/cds = (0,905)/gb = AB017112/gi = 4239973/ug = Mm.29666/ | P | 583.5 | P | 395.2 | P | 706.9 | P |
| | | len = 906/STRA = for | | | | | | | |
| 14 | 95722_at | Cluster Incl AB013137 Mus musculus mRNA for glutaredoxin, complete cds/cds = (49,372)/gb = AB013137/gi = 6451922/ | P | 1974 | P | 965.8 | P | 1784.3 | P |
| | | ug = Mm 29728/len = 1287/STRA = for | | | | | | | |
| 14 | 96840_at | Cluster Incl AI851243 UI-M-BH0-akf-b-01-0-UI s1 Mus musculus cDNA, 3 end/clone = UI-M-BH0-akf-b-01-0-UI/ | P | 932.7 | P | 731.6 | P | 1079.8 | P |
| | | clone_end = 3/gb = AI851243/gi = 5495149/ug = Mm.30017/len = 430/STRA = for | | | | | | | |
| 14 | 96870_at | Cluster Incl AI836740 UI-M-AJ0-aax-h-06-0-UI s1 Mus musculus cDNA, 3 end/clone = UI-M-AJ0-aax-h-06-0-UI/ | P | 3126.2 | P | 2770.6 | P | 3230.1 | P |
| | | clone_end = 3/gb = AI836740/gi = 5470958/ug = Mm.30065/len = 420/STRA = for | | | | | | | |
| 14 | 96900_at | Cluster Incl AW125480 UI-M-BH2 2-aqm-c-05-0-UI s1 Mus musculus cDNA, 3 end/clone = UI-M-BH2 2-aqm-c-05-0-UI/ | P | 1092.3 | P | 508.7 | P | 948 | P |
| | | clone_end = 3/gb = AW125480/gi = 6101010/ug = Mm 30114/len = 476/STRA = for | | | | | | | |
| 14 | 97485_at | Cluster Incl AI850953 UI-M-BH0-akd-e-11-0-UI s1 Mus musculus cDNA, 3 end/clone = UI-M-BH0-akd-e-11-0-UI/ | P | 1119.1 | P | 801.4 | P | 1042.4 | P |
| | | clone_end = 3/gb = AI850953/gi = 5494859/ug = Mm 30849/len = 328/STRA = for | | | | | | | |
| 14 | 97549_at | Cluster Incl L29468 Mouse cofilin Isoform mRNA, complete cds/cds = (131,631)/gb = L29468/gi = 498016/ug = Mm 3373/ | P | 425.2 | P | 281.3 | P | 373.5 | P |
| | | len = 2974/STRA = for | | | | | | | |
| 14 | 99141_at | Cluster Incl U09816 Mus musculus GM2 activator protein (Gm2a) mRNA, complete cds/cds = (56,637)/gb = U09616/ | P | 714.3 | P | 376.3 | P | 750.9 | P |
| | | gi = 540017/ug = Mm 4140/len = 2003/STRA = for | | | | | | | |
| 14 | 99160_s_at | Cluster Incl AW227647: up12b06.y1 Mus musculus cDNA, 5 end/clone = IMAGE-2654099/clone_end = 5/gb = AW227647/ | P | 754.1 | P | 524.7 | P | 959.2 | P |
| | | gi = 6556943/ug = Mm 41665/len = 410/STRA = for | | | | | | | |
| 14 | 99642_i_at | Cluster Incl X61232: Mouse mRNA for carboxypeptidase H/cds = (39,1472)/gb = X61232/gi = 50312/ug = Mm 43603/ | P | 1177.8 | P | 704.2 | P | 1115.3 | P |
| | | len = 1992/STRA = for | | | | | | | |
| 14 | 99643_f_at | Cluster Incl X61232: Mouse mRNA for carboxypeptidase H/cds = (39,1472)/gb = X61232/gi = 50312/ug = Mm 43603/ | P | 1322 | P | 898.3 | P | 1263.7 | P |
| | | len = 1992/STRA = for | | | | | | | |
| 14 | 100042_at | Cluster Incl AI837921 UI-M-AL0-abq-d-08-0-UI s1 Mus musculus cDNA, 3 end/clone = UI-M-AL0-abq-d-08-0-UI/ | P | 333.9 | P | 157.1 | P | 261.7 | P |
| | | clone_end = 3/gb = AI837921/gi = 5472134/ug = Mm 44240/len = 244/STRA = for | | | | | | | |
| 14 | 100120_at | Cluster Incl L17324 Nidogen/cds = (0,751)/gb = L17324/gi = 439608/ug = Mm 4691/len = 752/STRA = for | P | 582.4 | P | 328.8 | P | 661.3 | P |
| 14 | 100136_at | Cluster Incl M32017.Lysosomal membrane glycoprotein 2/cds = (0,692)/gb = M32017/gi = 198799/ug = Mm 486/len = 1067/ | P | 829.6 | P | 503.7 | P | 700.4 | P |
| | | STRA = for | | | | | | | |
| 14 | 100151_at | Cluster Incl L29441: Mus musculus mRNA, complete cds/cds = (69,1250)/gb = L29441/gi = 459889/ug = Mm 4962/len = 2029/ | P | 1384 | P | 958.9 | P | 1481.3 | P |
| | | STRA = for | | | | | | | |
| 14 | 100528_at | Cluster Incl AI852278 UI-M-BH0-aje-d-03-0-UI s1 Mus musculus cDNA, 3 end/clone = UI-M-BH0-aje-d-03-0-UI/ | P | 529.8 | P | 377.1 | P | 521 | P |
| | | clone_end = 3/gb = AI852278/gi = 5496184/ug = Mm 5203/len = 185/STRA = for | | | | | | | |
| 14 | 101008_at | Cluster Incl AB023485 Mus musculus mRNA for transcription factor CA150b, complete cds/cds = (4,3108)/gb = AB023485/ | P | 172.5 | P | 72.3 | P | 173.2 | P |
| | | gi = 6329165/ug = Mm 67998/len = 3526/STRA = for | | | | | | | |
| 14 | 101039_at | Cluster Incl X04647.Procollagen, type IV, alpha 2/cds = (0,2216)/gb = X04647/gi = 1022322/ug = Mm 6954/len = 3189/ | P | 1630.1 | P | 1661 | P | 1448.7 | P |
| | | STRA = rev | | | | | | | |
| 14 | 102049_at | Cluster Incl AJ001418 Mus musculus mRNA for pyruvate dehydrogenase kinase-like protein/cds = (116,1354)/ | P | 449.4 | P | 170.7 | P | 568.6 | P |
| | | gb = AJ001418/gi = 3005989/ug = Mm.10283/len = 3449/STRA = for | | | | | | | |
| 14 | 102340_at | Cluster Incl AA833077 uc94g09.r1 Mus musculus cDNA, 5 end/clone = IMAGE-1433344/clone_end = 5/gb = AA833077/ | P | 197.7 | P | 152.8 | P | 177.1 | P |
| | | gi = 2906805/ug = Mm.11116/len = 379/STRA = for | | | | | | | |
| 14 | 102346_at | Cluster Incl AI747755 ul21h12.x1 Mus musculus cDNA, 3 end/clone = IMAGE-2088263/clone_end = 3/gb = AI747755/ | P | 133.2 | P | 85.1 | P | 139.4 | P |
| | | gi = 5126019/ug = Mm 11218/len = 431/STRA = for | | | | | | | |
| 14 | 102370_at | Cluster Incl AA822174: vp36a09.r1 Mus musculus cDNA, 5 end/clone = IMAGE-1078744/clone_end = 5/gb = AA822174/ | P | 350.4 | P | 136.7 | P | 233 | P |
| | | gi = 2892042/ug = Mm.1187/len = 329/STRA = for | | | | | | | |
| 14 | 102827_at | Cluster Incl AW124633 UI-M-BH2 1-apw-c-02-0-UI s1 Mus musculus cDNA, 3 end/clone = UI-M-BH2.1-apw-c-02-0-UI/ | P | 592.5 | P | 343.1 | P | 522.3 | P |
| | | clone_end = 3/gb = AW124633/gi = 6100163/ug = Mm.14478/len = 421/STRA = for | | | | | | | |
| 14 | 103015_at | Cluster Incl U41465: B-cell leukemia/lymphoma 6/cds = (100,2223)/gb = U41465/gi = 1209719/ug = Mm 15811/len = 2373/ | P | 446.6 | P | 376.7 | P | 509.2 | P |
| | | STRA = for | | | | | | | |
| 14 | 103338_at | Cluster Incl AW120504 UI-M-BH1-ami-f-11-0-UI s2 Mus musculus cDNA, 3 end/clone = UI-M-BH1-ami-f-11-0-UI/ | P | 140.9 | P | 77.9 | P | 139 | P |
| | | clone_end = 3/gb = AW120504/gi = 6095837/ug = Mm.18113/len = 336/STRA = for | | | | | | | |
| 14 | 103359_at | Cluster Incl AI854144 UI-M-BH0-ajw-g-04-0-UI s1 Mus musculus cDNA, 3 end/clone = UI-M-BH0-ajw-g-04-0-UI/ | P | 353 | P | 310.3 | P | 359.8 | P |
| | | clone_end = 3/gb = AI854144/gi = 5498050/ug = Mm 18460/len = 515/STRA = for | | | | | | | |
| 14 | 103414_at | Cluster Incl AF049850 Mus musculus major histocompatibility locus class III region- complemenl C4 (C4) and cytochrome | P | 426.5 | P | 408.2 | P | 519.8 | P |
| | | P450 hydroxylase A (CYP21OH-A) genes, complete cds, sip pseudogene, complete sequence, NG6, SKI, and complement | | | | | | | |
| | | factor B (Bf) genes, comp/STRA = for | | | | | | | |
| 14 | 103796_at | Cluster Incl AF064071: Mus musculus apoptotic protease activating factor 1 mRNA, complete cds/cds = (585,4301)/ | P | 205.8 | P | 160.9 | P | 177 | P |
| | | gb = AF064071/gi = 3694812/ug = Mm.20836/len = 5152/STRA = for | | | | | | | |
| 14 | 103893_at | Cluster Incl AW048781 UI-M-BH1-amg-b-03-0-UI s1 Mus musculus cDNA, 3 end/clone = UI-M-BH1-amg-b-03-0-UI/ | P | 568.1 | P | 293.4 | P | 478.5 | P |
| | | clone_end = 3/gb = AW048781/gi = 5909310/ug = Mm.21295/len = 404/STRA = for | | | | | | | |
| 14 | 103901_at | Cluster Incl AW213777 uo42e09 x1 Mus musculus cDNA, 3 end/clone = IMAGE-2645224/clone_end = 3/gb = AW213777/ | P | 186.9 | P | 124.5 | P | 184 | P |
| | | gi = 6520028/ug = Mm.21331/len = 313/STRA = for | | | | | | | |
| 14 | 104288_at | Cluster Incl AI853265 UI-M-BH0-aji-c-07-0-UI s1 Mus musculus cDNA, 3 end/clone = UI-M-BH0-aji-c-07-0-UI/clone_end = 3/ | P | 542.3 | P | 365.1 | P | 485.3 | P |
| | | gb = AI853265/gi = 5497171/ug = Mm 22276/len = 433/STRA = for | | | | | | | |
| 14 | 104744_at | Cluster Incl AW046775 UI-M-BH1-alt-h-01-0-UI s1 Mus musculus cDNA, 3 end/clone = UI-M-BH1-alt-h-01-0-UI/ | P | 287.7 | P | 183.4 | P | 313.1 | P |
| | | clone_end = 3/gb = AW046775/gi = 5907304/ug = Mm 24707/len = 174/STRA = for | | | | | | | |
| 14 | 104758_at | Cluster Incl AA895984: vx52h06.r1 Mus musculus cDNA, 5 end/clone = IMAGE-1278875/clone_end = 5/gb = AA895984/ | P | 189 | P | 157.6 | P | 180.1 | P |
| | | gi = 3032377/ug = Mm.24794/len = 339/STRA = for | | | | | | | |
| 14 | 94354_at | Cluster Incl AI845514: UI-M-AO1-aek-d-04-0-UI.s1 Mus musculus cDNA, 3 end/clone = UI-M-AO1-aek-d-04-0-UI/ | P | 515.2 | P | 466 | P | 730.4 | P |
| | | clone_end = 3/gb = AI845514/gi = 5489420/ug = Mm 25221/len = 409/STRA = for | | | | | | | |
| 14 | 94359_at | Cluster Incl AI849556 UI-M-AH1-agr-b-05-0-UI s1 Mus musculus cDNA, 3 end/clone = UI-M-AH1-agr-b-05-0-UI/ | P | 1059.1 | P | 882.6 | P | 1091.9 | P |
| | | clone_end = 3/gb = AI849556/gi = 5493462/ug = Mm 25248/len = 396/STRA = rev | | | | | | | |
| 14 | 94374_at | Cluster Incl AI850378 UI-M-BG1-aig-d-01-0-UI s1 Mus musculus cDNA, 3 end/clone = UI-M-BG1-aig-d-01-0-UI/ | P | 387.3 | P | 256 | P | 363.4 | P |
| | | clone_end = 3/gb = AI850378/gi = 5494284/ug = Mm.25445/len = 449/STRA = for | | | | | | | |
| 14 | 94420_f_at | Cluster Incl AB000777: Cryptochrome 1 (photolyase-like)/cds = (19,1839)/gb = AB000777/gi = 1816438/ug = Mm 26237/ | P | 274.2 | P | 196.7 | P | 262.5 | P |
| | | len = 2450/STRA = for | | | | | | | |
| 14 | 95033_at | Cluster Incl AW049513 UI-M-BH1-anf-b-11-0-UI s1 Mus musculus cDNA, 3 end/clone = UI-M-BH1-anf-b-11-0-UI/ | P | 492.6 | P | 365.3 | P | 484.4 | P |
| | | clone_end = 3/gb = AW049513/gi = 5910042/ug = Mm 27535/len = 508/STRA = for | | | | | | | |
| 14 | 95564_at | Cluster Incl AI117859 uc41d02 r1 Mus musculus cDNA, 5 end/clone = IMAGE-1400547/clone_end = 5/gb = AI117859/ | P | 167 | P | 116.1 | P | 212.4 | P |
| | | gi = 3518183/ug = Mm.27785/len = 328/STRA = for | | | | | | | |
| 14 | 95601_at | Cluster Incl AW125420 UI-M-BH2 3-aqh-f-01-0-UI s1 Mus musculus cDNA, 3 end/clone = UI-M-BH2 3-aqh-f-01-0-UI/ | P | 723.3 | P | 574 | P | 662.8 | P |
| | | clone_end = 3/gb = AW125420/gi = 6100950/ug = Mm 27937/len = 524/STRA = for | | | | | | | |
| 14 | 96221_at | Cluster Incl AI606300 vm55g05 y1 Mus musculus cDNA, 5 end/clone = IMAGE-1002200/clone_end = 5/gb = AI606300/ | P | 173.1 | P | 77.4 | P | 214.5 | P |
| | | gi = 4615467/ug = Mm 28710/len = 406/STRA = for | | | | | | | |
| 14 | 96774_at | Cluster Incl AW047139 UI-M-BH1-alu-c-08-0-UI s1 Mus musculus cDNA, 3 end/clone = UI-M-BH1-alu-c-08-0-UI/ | P | 679.6 | P | 497.1 | P | 782.3 | P |
| | | clone_end = 3/gb = AW047139/gi = 5907668/ug = Mm 29048/len = 363/STRA = for | | | | | | | |
| 14 | 97406_at | Cluster Incl AW122258 UI-M-BH2 2-aov-g-09-0-UI.s1 Mus musculus cDNA, 3 end/clone = UI-M-BH2.2-aov-g-09-0-UI/ | P | 422.4 | P | 190.6 | P | 457.4 | P |
| | | clone_end = 3/gb = AW122258/gi = 6097727/ug = Mm 29914/len = 285/STRA = rev | | | | | | | |
| 14 | 97985_f_at | Cluster Incl AA709672 vt35h09 r1 Mus musculus cDNA, 5 end/clone = IMAGE-1165121/clone_end = 5/gb = AA709672/ | P | 853.7 | P | 411.4 | P | 762.6 | P |
| | | gi = 2719590/ug = Mm.31297/len = 364/STRA = for | | | | | | | |
| 14 | 98478_at | Cluster Incl U95826 Mus musculus cyclin G2 mRNA, complete cds/cds = (123,1157)/gb = U95826/gi = 2149913/ | P | 505.8 | P | 249.5 | P | 412 | P |
| | | ug = Mm 3527/len = 1280/STRA = for | | | | | | | |
| 14 | 98505_i_at | Cluster Incl D16333 Mouse mRNA for coproporphyrinogen oxidase, complete cds/cds = (401,1465)/gb = D16333/gi = 436576/ | P | 567 | P | 255.6 | P | 409.7 | P |
| | | ug = Mm 35820/len = 3071/STRA = for | | | | | | | |
| 14 | 98506_r_at | Cluster Incl D16333 Mouse mRNA for coproporphyrinogen oxidase, complete cds/cds = (401,1465)/gb = D16333/gi = 436576/ | P | 353.4 | P | 196.9 | P | 214.7 | P |
| | | ug = Mm 35820/len = 3071/STRA = for | | | | | | | |
| 14 | 99064_at | Cluster Incl AF026469: Ubiquitous nuclear protein/cds = (50,2938)/gb = AF026469/gi = 2655203/ug = Mm 3974/len = 3329/ | P | 865.1 | P | 728.4 | P | 1079.5 | P |
| | | STRA = for | | | | | | | |
| 14 | 99500_at | Cluster Incl U13174: Solute carrier family 12, member 2/cds = (129,3746)/gb = U13174/gi = 3885309/ug = Mm 4168/len = 6504/ | P | 611.9 | P | 485.6 | P | 587.3 | P |
| | | STRA = rev | | | | | | | |
| 14 | 99532_at | Cluster Incl D78382: Mouse DNA for tob family, complete cds/cds = (0,1091)/gb = D78382/gi = 1469158/ug = Mm 4218/ | P | 1349.6 | P | 961.9 | P | 1181.2 | P |
| | | len = 1092/STRA = for | | | | | | | |
| 14 | 99960_at | Cluster Incl U18310 SAPK/Erk/kinase 1/cds = (28,1221)/gb = U18310/gi = 1754530/ug = Mm 4412/len = 2292/STRA = for | P | 368.6 | P | 341.3 | P | 433.6 | P |
| 14 | 100034_at | Cluster Incl U54705: Mus musculus tumor supressor maspin mRNA, complete cds/cds = (189,1316)/gb = U54705/ | P | 1115.2 | P | 840.8 | P | 1305.6 | P |
| | | gi = 1490512/ug = Mm 4622/len = 1432/STRA = for | | | | | | | |
| 14 | 100475_at | Cluster Incl D63902: Zinc finger protein 147/cds = (9,1913)/gb = D63902/gi = 1088466/ug = Mm.4973/len = 5475/STRA = rev | P | 434 | P | 147.9 | P | 198.8 | P |
| 14 | 100482_at | Cluster Incl AI836408 UI-M-APO-abi-g-08-0-UI s1 Mus musculus cDNA, 3 end/clone = UI-M-AP0-abi-g-08-0-UI/ | P | 855.2 | P | 716 | P | 839.4 | P |
| | | clone_end = 3/gb = AI836408/gi = 5470621/ug = Mm 5018/len = 277/STRA = rev | | | | | | | |
| 14 | 100978_at | Cluster Incl U62105 FSHD region gene 1/cds = (116,892)/gb = U62105/gi = 1762524/ug = Mm 67/len = 1022/STRA = for | P | 305 | P | 217.5 | P | 222.3 | P |
| 14 | 102870_at | Cluster Incl AW125272: UI-M-BH2 1-aps-h-06-0-UI s1 Mus musculus cDNA, 3 end/clone = UI-M-BH2 1-aps-h-06-0-UI/ | P | 572.4 | P | 222.5 | P | 327.7 | P |
| | | clone_end = 3/gb = AW125272/gi = 6100802/ug = Mm 14787/len = 349/STRA = for | | | | | | | |
| 14 | 102912_at | Cluster Incl AI265115 uk03d02 y1 Mus musculus cDNA, 5 end/clone = IMAGE-1958307/clone_end = 5/gb = AI265115/ | P | 512.7 | P | 401.9 | P | 495.9 | P |
| | | gi = 3883273/ug = Mm 1610/len = 473/STRA = for | | | | | | | |
| 14 | 103263_at | Cluster Incl AF079755 Mus musculus enhancer of polycomb (Epc1) mRNA, complete cds/cds = (220,2511)/gb = AF079765/ | P | 501.5 | P | 399.2 | P | 519.8 | P |
| | | gi = 3757891/ug = Mm 20828/len = 3895/STRA = for | | | | | | | |
| 14 | 103456_at | Cluster Incl AI841800 UI-M-AN0-acl-g-06-0-UI s1 Mus musculus cDNA, 3 end/clone = UI-M-AN0-acl-g-06-0-UI/ | P | 785.1 | P | 569 | P | 762.2 | P |
| | | clone_end = 3/gb = AI841800/gi = 5476013/ug = Mm 21657/len = 371/STRA = for | | | | | | | |
| 14 | 103457_at | Cluster Incl AF083464 Mus musculus DNA polymerase zeta catalytic subunit mRNA, complete cds/cds = (48,9416)/ | P | 480.9 | P | 356.2 | P | 475.2 | P |
| | | gb = AF083464/gi = 4079830/ug = Mm.2167/len = 10404/STRA = for | | | | | | | |
| 14 | 103697_at | Cluster Incl AW061234 UI-M-BH1-amj-b-11-0-UI s2 Mus musculus cDNA, 3 end/clone = UI-M-BH1-emj-b-11-0-UI/ | P | 498.7 | P | 313.7 | P | 531.6 | P |
| | | clone_end = 3/gb = AW061234/gi = 6008985/ug = Mm 24003/len = 462/STRA = for | | | | | | | |
| 14 | 103773_at | Cluster Incl AW047874 UI-M-BH1-ala-g-02-0-UI s1 Mus musculus cDNA, 3 end/clone = UI-M-BH1-als-g-02-0-UI/ | P | 291.5 | P | 301 | P | 304.6 | P |
| | | clone_end = 3/gb = AW047874/gi = 5908403/ug = Mm.24787/len = 433/STRA = for | | | | | | | |
| 14 | 104033_at | Cluster Incl AI841996 UI-M-AH0-ada-b-03-0-UI s1 Mus musculus cDNA, 3 end/clone = UI-M-AH0-ada-b-03-0-UI/ | P | 494.6 | P | 211.7 | P | 365 | P |
| | | clone_end = 3/gb = AI841996/gi = 5476209/ug = Mm 27051/len = 401/STRA = for | | | | | | | |
| 14 | 104189_at | Cluster Incl AI122079 uc46e01.r1 Mus musculus cDNA, 5 end/clone = IMAGE-1401048/clone_end = 5/gb = AI122079/ | P | 2341. | P | 250.2 | P | 285.2 | P |
| | | gi = 3522403/ug = Mm 27463/len = 362/STRA = for | | | | | | | |
| 14 | 104403_at | Cluster Incl AI854118 UI-M-BH0-ajw-d-12-0-UI s1 Mus musculus cDNA, 3 end/clone = UI-M-BH0-ajw-d-12-0-UI/ | P | 421.8 | P | 193.4 | P | 389.9 | P |
| | | clone_end = 3/gb = AI854118/gi = 5498024/ug = Mm 28691/len = 504/STRA = rev | | | | | | | |
| 14 | 104513_at | Cluster Incl AA688938: vs04f09.r1 Mus musculus cDNA, 5 end/clone = IMAGE-1137257/clone_end = 5/gb = AA688938/ | P | 429.2 | P | 274.2 | P | 582.7 | P |
| | | gi = 2678367/ug = Mm 3090/len = 197/STRA = for | | | | | | | |
| 14 | 92217_s_at | Cluster Incl U05265 Glycoprotein 49 B/cds = (43,1050)/gb = U05265/gi = 475446/ug = Mm 34408/len = 1420/STRA = for | P | 474.2 | H | 272.1 | P | 496 | P |
| 14 | 92384_at | Cluster Incl X74351: Xeroderma pigmentosum, complementation group A/cds = (91,909)/gb = X74351/gi = 440584/ | P | 166.7 | P | 134 | P | 196.8 | P |
| | | ug = Mm.4049/len = 938/STRA = for | | | | | | | |
| 14 | 92791_at | Cluster Incl AA518588 vh94g10 r1 Mus musculus cDNA, 5 end/clone = IMAGE-901986/clone_end = 5/gb = AA518586/ | P | 350.7 | P | 346.5 | P | 400.9 | P |
| | | gi = 2258612/ug = Mm. 45985/len = 255/STRA = rev | | | | | | | |
| 14 | 93626_at | Cluster Incl AF103875: Mus musculus placenta-specific ATP-binding cassette transporter (Abcp) mRNA, partial cds/ | P | 237.4 | P | 157.9 | P | 212.3 | P |
| | | cds = (0,443)/gb = AF103875/gi = 4185797/ug = Mm.7458/len = 502/STRA = for | | | | | | | |
| 14 | 95387_f_at | Cluster Incl AA266467: mz67g09 r1 Mus musculus cDNA, 5 end/clone = IMAGE-718528/clone_end = 5/gb = AA266467/ | P | 407.7 | P | 300.7 | P | 420.4 | P |
| | | gi = 1902637/ug = Mm 23470/len = 372/STRA = rev | | | | | | | |
| 14 | 95897_at | Cluster Incl AW061228 UI-M-BH1-amj-a-12-0-UI s2 Mus musculus cDNA, 3 end/clone = UI-M-BH1-amj-a-12-0-UI/ | P | 310.7 | P | 165.5 | P | 283.5 | P |
| | | clone_end = 3/gb = AW061228/gi = 6008979/ug = Mm.24179/len = 475/STRA = rev | | | | | | | |
| 14 | 98855_r_at | Cluster Incl AA014745 mh18b05 r1 Mus musculus cDNA, 5 end/clone = IMAGE-442833/clone_end = 5/gb = AA014745/ | P | 295.3 | P | 107.3 | P | 270.8 | P |
| | | gi = 1475626/ug = Mm 5096/len = 189/STRA = for | | | | | | | |
| 14 | 95785_s_at | Cluster Incl Y13361 RAB7, member RAS oncogene family, pseudogene 1/cds = (0,623)/gb = Y13361/gi = 2168154/ | P | 396.5 | P | 227.7 | P | 478.9 | P |
| | | ug = Mm 87807/len = 624/STRA = for | | | | | | | |
| 14 | 101221_at | Cluster Incl C76746 C76746 Mus musculus cDNA, 3 end/clone = J0018D06/clone_end = 3/gb = C76746/gi = 2517076/ | P | 294.3 | P | 184.3 | P | 239.5 | P |
| | | ug = Mm.5529/len = 574/STRA = rev | | | | | | | |
| 14 | 93836_at | Cluster Incl AF041054 Mus musculus E1B 19K/Bcl-2-binding protein homolog (Nip3) mRNA, nuclear gene encoding | P | 294 | P | 265.8 | A | 379.2 | P |
| | | mitochondrial protein, complete cds/cds = (83,646)/gb = AF041054/gi = 2773344/ug = Mm 2159/len = 1311/STRA = for | | | | | | | |
| 14 | 96106_at | Cluster Incl AW049122 UI-M-BH1-amt-h-11-0-UI s1 Mus musculus cDNA, 3 end/clone = UI-M-BH1-amt-h-11-0-UI/ | P | 189.5 | P | 142.6 | P | 235.6 | P |
| | | clone_end = 3/gb = AW049122/gi = 5909651/ug = Mm 26903/len = 513/STRA = for | | | | | | | |
| 14 | 96679_at | Cluster Incl AW120711: UI-M-BH2 3-anz-e-10-0-UI.s1 Mus musculus cDNA, 3 end/clone = UI-M-BH2.3-anz-e-10-0-UI/ | P | 50.8 | P | 43.8 | P | 63.9 | P |
| | | clone_end = 3/gb = AW120711/gi = 6096009/ug = Mm.27432/len = 497/STRA = for | | | | | | | |
| 14 | 96884_at | Cluster Incl AI847631 UI-M-AP1-agg-e-12-0-UI s1 Mus musculus cDNA, 3 end/clone = UI-M-AP1-agg-e-12-0-UI/ | P | 443.3 | P | 356 | P | 481 | P |
| | | clone_end = 3/gb = AI847631/gi = 5491537/ug = Mm.30081/len = 334/STRA = for | | | | | | | |
| 14 | 97475_at | Cluster Incl D38517.Mouse mRNA for Dhm1 protein, complete cds/cds = (0,2843)/gb = D38517/gi = 1060920/ug = Mm 3065/ | P | 123.9 | P | 97.3 | P | 145.4 | P |
| | | len = 2844/STRA = for | | | | | | | |
| 14 | 103673_at | Cluster Incl M57891: Complement component 2 (wthin H-2S)/cds = (52,2334)/gb = M57891/gi = 192436/ug = Mm 2081/ | P | 218.6 | P | 157.9 | P | 253.2 | P |
| | | len = 2587/STRA = for | | | | | | | |
| 14 | 104569_at | Cluster Incl AW122024 UI-M-BH2.2-aoo-a-11-0-UI s1 Mus musculus cDNA, 3 end/clone = UI-M-BH2 2-aoo-a-11-0-UI/ | P | 296.9 | P | 211.9 | P | 280.8 | P |
| | | clone_end = 3/gb = AW122024/gi = 6097430/ug = Mm 2389/len = 498/STRA = for | | | | | | | |
| 14 | 104600_at | Cluster Incl AI841692 UI-M-AL0-abo-a-02-0-UI s1 Mus musculus cDNA, 3 end/clone = UI-M-AL0-abo-a-02-0-UI/ | P | 89.3 | P | 82.9 | P | 91 | P |
| | | clone_end = 3/gb = AI841692/gi = 5475905/ug = Mm 24084/len = 415/STRA = for | | | | | | | |
| 14 | 95031_at | Cluster Incl AW060212: UI-M-BH1-amu-f-06-0-UI.s1 Mus musculus cDNA, 3 end/clone = UI-M-BH1-amu-f-06-0-UI/ | P | 105.7 | P | 70.6 | P | 94.3 | P |
| | | clone_end = 3/gb = AW060212/gi = 6007963/ug = Mm 27520/len = 378/STRA = for | | | | | | | |
| 14 | 97972_at | Cluster Incl AB012161 Mus musculus gene for KF-1, complete cds/cds = (0,2051)/gb = AB012161/gi = 4521316/ug = Mm 310/ | P | 175.1 | P | 40.5 | M | 192.8 | P |
| | | len = 2052/STRA = for | | | | | | | |
| 14 | 99018_at | Cluster Incl AA874446 vx09g08 r1 Mus musculus cDNA, 5 end/clone = IMAGE-1263998/clone_end = 5/gb = AA874446/ | P | 87.1 | P | 52.3 | P | 73.6 | P |
| | | gi = 2979135/ug = Mm 385/len = 511/STRA = for | | | | | | | |
| 14 | 99512_at | Cluster Incl AI852647: UI-M-BH0-aiu-h-07-0-UI s1 Mus musculus cDNA, 3 end/clone = UI-M-BH0-aiu-h-07-0-UI/ | P | 684.1 | P | 569.6 | P | 583 | P |
| | | clone_end = 3/gb = AI852647/gi = 5496553/ug = Mm 41858/len = 379/STRA = for | | | | | | | |
| 14 | 103678_at | Cluster Incl AI841139 UI-M-AH0-acz-g-08-0-UI s1 Mus musculus cDNA, 3 end/clone = UI-M-AH0-acz-g-08-0-UI/ | P | 446.6 | P | 250.7 | P | 414 | A |
| | | clone_end = 3/gb = AI841139/gi = 5475352/ug = Mm 23879/len = 450/STRA = for | | | | | | | |
| 14 | 104671_at | Cluster Incl D88994 AMP deaminase 3/cds = (5,2083)/gb = D88994/gi = 3493348/ug = Mm 3238/len = 2127/STRA = for | P | 188.4 | P | 179.3 | A | 297.7 | P |
| 14 | 93148_at | Cluster Incl AI851013 UI-M-BH0-ajv-c-07-0-UI s1 Mus musculus cDNA, 3 end/clone = UI-M-BH0-ajv-c-07-0-UI/ | P | 195.8 | P | 112.7 | P | 188 | A |
| | | clone_end = 3/gb = AI851013/gi = 5494919/ug = Mm 5231/len = 485/STRA = for | | | | | | | |
| 14 | 98802_at | Cluster Incl D30782 Mouse mRNA for epiregulin, complete cds/cds = (138,626)/gb = D30782/gi = 1255631/ug = Mm 4791/ | P | 159.2 | P | 64.8 | P | 146.7 | P |
| | | len = 4141/STRA = for | | | | | | | |
| 14 | 94066_at | Cluster Incl AW123517 UI-M-BH2 1-aqb-c-09-0-UI s1 Mus musculus cDNA, 3 end/clone = UI-M-BH2 1-aqb-c-09-0-UI/ | P | 364.1 | P | 232.5 | A | 442.8 | M |
| | | clone_end = 3/gb = AW123517/gi = 6099047/ug = Mm 22086/len = 244/STRA = for | | | | | | | |
| 14 | 95093_at | Cluster Incl AI035334 ue18f10x1 Mus musculus cDNA, 3 end/clone = IMAGE-1480747/clone_end = 3/gb = AI035334/ | P | 224.8 | P | 219.9 | P | 267.5 | P |
| | | gi = 3259044/ug = Mm.29303/len = 182/STRA = for | | | | | | | |
| 14 | 103011_at | Cluster Incl L36831: Transcriptional regulator, SIN3 yeast homolog A/cds = (78,3926)/gb = L36831/gi = 556218/ | P | 361.9 | P | 270.7 | P | 423.7 | P |
| | | ug = Mm 15755/len = 4721/STRA = for | | | | | | | |
| 14 | 101370_at | Cluster Incl U20619.Mus musculus serine-rich RNA polymerase suppressor protein 1 (SRP1) mRNA, complete sequence/ | P | 262 | P | 97 | A | 273.9 | A |
| | | cds = UNKNOWN/gb = U20619/gi = 3046878/ug = Mm 6952/len = 3882/STRA = for | | | | | | | |
| 14 | 102916_s_at | Cluster Incl AB010266 Mus musculus Creb-rp, Tnx and Cyp21 genes for cAMP response element binding protein-related | P | 568.5 | P | 333.8 | P | 443.6 | P |
| | | protein, lenascin-X and steroid 21-hydroxylase, partial cds and complete sequences/cds = (0,12344)/gb = AB010266/ | | | | | | | |
| | | gi = 2804287/ug = Mm.16163/len = /STRA = for | | | | | | | |
| 14 | 102922_at | Cluster Incl AI851387.UI-M-BH0-akh-g-12-0-UI s1 Mus musculus cDNA, 3 end/clone = UI-M-BH0-akh-g-12-0-UI/ | P | 99.5 | P | 68 | P | 97.2 | P |
| | | clone_end = 3/gb = AI851387/gi = 5495293/ug = Mm 1630/len = 436/STRA = rev | | | | | | | |
| 14 | 102966_at | Cluster Incl AA934166 vt48h11.r1 Mus musculus cDNA, 5 end/clone = IMAGE-1166373/clone_end = 5/gb = AA934166/ | P | 232.6 | P | 201.9 | P | 246.6 | M |
| | | gi = 3091323/ug = Mm 1819/len = 321/STRA = for | | | | | | | |
| 14 | 103254_at | Cluster Incl AW049897: UI-M-BH1-anr-h-08-0-UI s1 Mus musculus cDNA, 3 end/clone = UI-M-BH1-anr-h-08-0-UI/ | P | 361.2 | P | 95.6 | P | 307.8 | P |
| | | clone_end = 3/gb = AW049897/gi = 5910426/ug = Mm 20488/len = 275/STRA = for | | | | | | | |
| 14 | 104661_at | Cluster Incl AF031164: AF031164 Mus musculus cDNA/gb = AF031164/gi = 4887815/ug = Mm.31787/len = 800/STRA = for | A | 160.8 | P | 125.8 | P | 144.7 | P |
| 14 | 93846_at | Cluster Incl U82324 Mus musculus A6 protein tyrosine kinase homolog mRNA, complete cds/cds = (56,1108)/gb = U82324/ | P | 107.2 | P | 87.4 | A | 118.9 | P |
| | | gi = 1769576/ug = Mm 7958/len = 1200/STRA = for | | | | | | | |
| 14 | 97172_s_at | Cluster Incl D86037: Sulfonylurea receptor 2/cds = (161,4801)/gb = D86037/gi = 4996851/ug = Mm 35070/len = 6210/ | A | 86.7 | P | 67.3 | P | 75 | P |
| | | STRA = for | | | | | | | |
| 14 | 96692_at | Cluster Incl AA759408 vw64c05.r1 Mus musculus cDNA, 5 end/clone = IMAGE-1248584/clone_end = 5/gb = AA759408/ | P | 384.6 | P | 199.8 | P | 408 | A |
| | | gi = 2807202/ug = Mm.27523/len = 424/STRA = for | | | | | | | |
| 14 | 101538_i_at | Cluster Incl AW226939 um64a07.y1 Mus musculus cDNA, 5 end/clone = IMAGE-2286132/clone_end = 5/gb = AW226939/ | P | 242.2 | P | 210.7 | A | 260.9 | P |
| | | gi = 6556235/ug = Mm 88078/len = 486/STRA = rev | | | | | | | |
| 14 | 102836_at | Cluster Incl U96724: Putative phosphatase/cds = (111,1517)/gb = U96724/gi = 2121245/ug = Mm 1458/len = 2684/STRA = for | P | 329.8 | P | 153.4 | A | 267.5 | A |
| 14 | 94980_at | Cluster Incl AI006319: ua70b05.r1 Mus musculus cDNA, 5 end/clone = IMAGE-1362801/clone_end = 5/gb = AI006319/ | P | 143.2 | P | 25.3 | A | 183 | P |
| | | gi = 3215928/ug = Mm.27275/len = 381/STRA = for | | | | | | | |
| 14 | 103716_at | Cluster Incl AI851218: UI-M-BH0-ajx-g-06-0-UI s1 Mus musculus cDNA, 3 end/clone = UI-M-BH0-ajx-g-06-0-UI/ | A | 256.9 | P | 209.3 | P | 324 | P |
| | | clone_end = 3/gb = AI851218/gi = 5495124/ug = Mm.24160/len = 306/STRA = rev | | | | | | | |
| 14 | 103967_at | Cluster Incl AI551105 vx37f03 x1 Mus musculus cDNA, 3 end/clone = IMAGE-1277405/clone_end = 3/gb = AI551105/ | P | 71.9 | P | 27.6 | A | 75.2 | P |
| | | gi = 4483468/ug = Mm.25678/len = 390/STRA = for | | | | | | | |
| 14 | 95292_at | Cluster Incl AA189389 mt85f02 r1 Mus musculus cDNA, 5 end/clone = IMAGE-636699/clone_end = 5/gb = AA189389/ | P | 86.1 | P | 40.3 | A | 90.2 | P |
| | | gi = 1776990/ug = Mm 1919/len = 451/STRA = for | | | | | | | |
| 14 | 97253_at | Cluster Incl AI854343 UI-M-BG1-aic-c-07-0-UI s1 Mus musculus cDNA, 3 end/clone = UI-M-BG1-aic-c-07-0-UI/ | P | 158.9 | P | 90.3 | M | 178.3 | P |
| | | clone_end = 3/gb = AI854343/gi = 5498249/ug = Mm 27872/len = 305/STRA = for | | | | | | | |
| 14 | 102032_at | Cluster Incl AW060819: UI-M-BH1-amx-b-03-0-UI.s1 Mus musculus cDNA, 3 end/clone = UI-M-BH1-amx-b-03-0-UI/ | A | 327.5 | P | 160.8 | P | 385.4 | A |
| | | clone_end = 3/gb = AW060819/gi = 6008570/ug = Mm 10153/len = 507/STRA = for | | | | | | | |
| 14 | 102780_at | Cluster Incl Z31362: M musculus (Balb/C) Tx01 mRNA/cds = (0,159)/gb = Z31362/gi = 467582/ug = Mm 1364/len = 228/ | P | 191.9 | P | 118.4 | A | 274.8 | A |
| | | STRA = for | | | | | | | |
| 14 | 102852_at | Cluster Incl M31131: Cadhenn 2/cds = (332,3052)/gb = M31131/gi = 192327/ug = Mm 14897/len = 4321/STRA = for | A | 64.6 | P | 17.7 | A | 94.6 | P |
| 14 | 104065_at | Cluster Incl AW212878 uo68g01 x1 Mus musculus cDNA, 3 end/clone = IMAGE-2647728/clone_end = 3/gb = AW212878/ | P | 71.2 | P | 39.4 | A | 73.8 | P |
| | | gi = 6519026/ug = Mm 21596/len = 280/STRA = for | | | | | | | |
| 14 | 104067_at | Cluster Incl AI648999 uk33h10.x1 Mus musculus cDNA, 3 end/clone = IMAGE-1970851/clone_end = 3/gb = AI648999/ | P | 151 | P | 58.9 | A | 155.6 | A |
| | | gi = 4729833/ug = Mm.21608/len = 579/STRA = for | | | | | | | |
| 14 | 104144_at | Cluster Incl AW124369 UI-M-BH2 1-ape-a-04-0-UI.s1 Mus musculus cDNA, 3 end/clone = UI-M-BH2 1-ape-a-04-0-UI/ | M | 356.4 | P | −136.3 | A | 295.6 | M |
| | | clone_end = 3/gb = AW124369/gi = 6099899/ug = Mm 22147/len = 214/STRA = for | | | | | | | |
| 14 | 97984_i_at | Cluster Incl AA709672: vt35h09 r1 Mus musculus cDNA, 5 end/clone = IMAGE-1165121/clone_end = 5/gb = AA709672/ | P | 362.3 | P | 113.5 | A | 273.7 | P |
| | | gi = 2719590/ug = Mm.31297/len = 364/STRA = for | | | | | | | |
| 14 | 104215_at | Cluster Incl AA592069.vo14b08.r1 Mus musculus cDNA, 5 end/clone = IMAGE-1049847/clone_end = 5/gb = AA592069/ | P | 86.5 | P | 40.9 | A | 99.2 | M |
| | | gi = 2405732/ug = Mm.27843/len = 329/STRA = for | | | | | | | |
| 14 | 98297_at | Cluster Incl AF110818 Mus musculus orphan G protein-coupled receptor FEX mRNA, complete cds/cds = (0,2723)/ | P | 170.6 | P | −62.3 | A | 168.1 | A |
| | | gb = AF110818/gi = 4262545/ug = Mm.42103/len = 3115/STRA = for | | | | | | | |
| 14 | 93054_at | Cluster Incl AI846368 UI-M-AN1-afa-e-09-0-UI s1 Mus musculus cDNA, 3 end/clone = UI-M-AN1-afa-e-09-0-UI/ | P | 178.9 | P | 99.4 | A | 176.3 | A |
| | | clone_end = 3/gb = AI846368/gi = 5490274/ug = Mm 15351/len = 385/STRA = for | | | | | | | |
| 14 | 94324_f_at | Cluster Incl U49878.3-hydroxy-3-methylglutaryl-coenzyme A lyase/cds = (34,1011)/gb = U49878/gi = 1292951/ | P | 639.1 | P | 585.3 | A | 685.8 | P |
| | | ug = Mm 22668/len = 1440/STRA = for | | | | | | | |
| 14 | 96912_s_at | Cluster Incl X15591 Cytotoxic T lymphocyte-associated protein 2 alpha/cds = (6,416)/gb = X15591/gi = 50588/ug = Mm.30144/ | P | 655.7 | P | 255.1 | A | 369.7 | A |
| | | len = 664/STRA = for | | | | | | | |
| 14 | 99166_at | Cluster Incl AI845963 UI-M-AP1-agl-a-09-0-UI s1 Mus musculus cDNA, 3 end/clone = UI-M-AP1-agl-a-09-0-UI/ | P | 342.1 | A | 223.5 | P | 359.1 | A |
| | | clone_end = 3/gb = AI845963/gi = 5489869/ug = Mm 41683/len = 223/STRA = for | | | | | | | |
| 14 | 96811_at | Cluster Incl AW049806 UI-M-BH1-anm-g-08-0-UI s1 Mus musculus cDNA, 3 end/clone = UI-M-BH1-anm-g-08-0-UI/ | P | 138.5 | A | 66.1 | A | 203.4 | A |
| | | clone_end = 3/gb = AW04986/gi = 5910335/ug = Mm 29274/len = 457/STRA = for | | | | | | | |
| 14 | 99510_at | Cluster Incl X59274 M musculus mRNA brain protein kinase C beta-1/cds = UNKNOWN/gb = X59274/gi = 50185/ | A | 186.7 | P | 66.6 | A | 276.5 | A |
| | | ug = Mm 4182/len = 2493/STRA = for | | | | | | | |
| 14 | 101848_g_at | Cluster Incl AF040242 untitled/cds = (0,240)/gb = AF040242/gi = 3309551/ug = Mm 8551/len = 241/STRA = for | A | 164.7 | P | 98.9 | P | 211.2 | A |
| 14 | 100321_f_at | Cluster Incl AA407120 EST02104 Mus musculus cDNA, 5 end/clone = C0017F03/clone_end = 5/gb = AA407120/ | A | 45.3 | P | 10.6 | A | 37.3 | P |
| | | gi = 2067019/ug = Mm 87656/len = 359/STRA = for | | | | | | | |
| 14 | 101804_at | Cluster Incl AF032715 Mus musculus type II peroxiredoxin protein 3 (PrxII-3) pseudogene, complete sequence/ | P | 244 | P | 131.3 | A | 181.5 | A |
| | | cds = (0,452)/gb = AF032715/gi = 3603232/ug = Mm.57146/len = 453/STRA = for | | | | | | | |
| 14 | 94491_at | Cluster Incl AW123194: UI-M-BH2 1-apg-c-01-0-UI s1 Mus musculus cDNA, 3 end/clone = UI-M-BH2.1-apg-c-01-0-UI/ | A | 157.9 | P | 94.6 | P | 125.7 | P |
| | | clone_end = 3/gb = AW123194/gi = 6098764/ug = Mm 28912/len = 509/STRA = for | | | | | | | |
| 14 | 95094_g_at | Cluster Incl AI035334 ue18f10 x1 Mus musculus cDNA, 3 end/clone = IMAGE-1480747/clone_end = 3/gb = AI035334/ | A | 139.3 | P | 73.4 | A | 111.4 | A |
| | | gi = 3259044/ug = Mm.29303/len = 182/STRA = for | | | | | | | |
| 14 | 104368_at | Cluster Incl U51204: Mus musculus APC-binding protein EB2 mRNA, partial cds/cds = (0,677)/gb = U51204/gi = 1256435/ | P | 167.5 | A | 63 | P | 139 | M |
| | | ug = Mm 22628/len = 1540/STRA = for | | | | | | | |
| 14 | 96770_at | Cluster Incl AA607761 vo08f08 r1 Mus musculus cDNA, 5 end/clone = IMAGE-1040871/clone_end = 5/gb = AA607761/ | P | 136.5 | A | −86.6 | A | 361.8 | P |
| | | gi = 2455196/ug = Mm 29017/len = 491/STRA = for | | | | | | | |
| 14 | 100507_at | Cluster Incl Y09257 M musculus mRNA for NOV protein/cds = (32,1096)/gb = Y09257/gi = 1666472/ug = Mm 5167/len = 2364/ | A | 223.7 | P | −145.4 | A | 358.2 | P |
| | | STRA = for | | | | | | | |
| 14 | 102965_at | Cluster Incl AW121646 UI-M-BH2 3-aoc-b-03-0-UI s1 Mus musculus cDNA, 3 end/clone = UI-M-BH2 3-aoc-b-03-0-UI/ | P | 400.2 | P | 245.4 | A | 352.4 | M |
| | | clone_end = 3/gb = AW121646/gi = 6096979/ug = Mm.1806/len = 512/STRA = for | | | | | | | |
| 14 | 93609_at | Cluster Incl AA797709 vw31c03 r1 Mus musculus cDNA, 5 end/clone = IMAGE-1245412/clone_end = 5/gb = AA797709/ | P | 98 | A | 26.7 | A | 52.8 | A |
| | | gi = 2860664/ug = Mm 7265/len = 464/STRA = for | | | | | | | |
| 14 | 96528_at | Cluster Incl D18912 MUSGS01097 Mus musculus cDNA, 3 end/clone = mc0837/clone_end = 3/gb = D18912/gi = 1089548/ | A | 40.6 | P | 32.4 | M | 66 | P |
| | | ug = Mm 27078/len = 254/STRA = rev | | | | | | | |
| 14 | 97710_f_at | Cluster Incl AI425990: mg38e07 x1 Mus musculus cDNA, 3 end/clone = IMAGE-426084/clone_end = 3/gb = AI425990/ | A | 53.5 | M | 14.4 | A | 39.7 | P |
| | | gi = 4271921/ug = Mm 38264/len = 407/STRA = rev | | | | | | | |
| 15 | AFFX- | E Coli BioD 3 prime | | | | | | | |
| | BioDn-3_at | | | | | | | | |
| 15 | AFFX- | Cre 5 prime | | | | | | | |
| | CreX-5_at | | | | | | | | |
| 15 | AFFX- | Cre 3 prime | | | | | | | |
| | CreX-3_at | | | | | | | | |
| 15 | AFFX-b- | M12481 Mouse cytoplasmic beta-actin mRNA (_5, _M, _3 represent transcript regions 5 prime, | P | 2660.1 | P | 5084.6 | P | 4250.3 | P |
| | ActinMur/ | Middle, and 3 prime respectively) | | | | | | | |
| | M12481_5_at | | | | | | | | |
| 15 | AFFX-b- | M12481 Mouse cytoplasmic beta-actin mRNA (_5, _M, _3 represent transcript regions 5 prime, | P | 3575.4 | P | 6085.1 | P | 5333.1 | P |
| | ActinMur/ | Middle, and 3 prime respectively) | | | | | | | |
| | M12481_{—} | | | | | | | | |
| | M_at | | | | | | | | |
| 15 | AFFX-b- | M12481 Mouse cytoplasmic beta-actin mRNA (_5, _M, _3 represent transcript regions 5 prime, | P | 2710.7 | P | 4018.9 | P | 4494.2 | P |
| | ActinMur/ | Middle, and 3 prime respectively) | | | | | | | |
| | M12481_3_at | | | | | | | | |
| 15 | AFFX- | M32599 Mouse glyceraldehyde-3-phosphate dehydrogenase mRNA, complete cds (_5, _M, _3 represent | P | 3898.4 | P | 6787.5 | P | 5413.8 | P |
| | GapdhMur/ | transcript regions 5 prime, Middle, and 3 prime respectively) | | | | | | | |
| | M32599_5_at | | | | | | | | |
| 15 | AFFX- | M32599 Mouse glyceraldehyde-3-phosphate dehydrogenase mRNA, complete cds (_5, _M, _3 represent | P | 3541.8 | P | 6515.1 | P | 5597.2 | P |
| | GapdhMur/ | transcript regions 5 prime, Middle, and 3 prime respectively) | | | | | | | |
| | M32599_{—} | | | | | | | | |
| | M_at | | | | | | | | |
| 15 | AFFX- | M32599 Mouse glyceraldehyde-3-phosphate dehydrogenase mRNA, complete cds (_5, _M, _3 represent | P | 2670.9 | P | 5092.9 | P | 3962.9 | P |
| | GapdhMur/ | transcript regions 5 prime, Middle, and 3 prime respectively) | | | | | | | |
| | M32599_{—} | | | | | | | | |
| | 3_at | | | | | | | | |
| 15 | AFFX- | X63136 M musculus DNA for intragenic sequence Including B2 element | P | 1762.2 | P | 2981.7 | P | 2803.5 | P |
| | MUR_{—} | | | | | | | | |
| | b2_at | | | | | | | | |
| 15 | AFFX- | U01310 Mus musculus C57/Black6 BC1 scRNA | P | 3170.3 | P | 4853.7 | P | 4114.2 | P |
| | MURINE_{—} | | | | | | | | |
| | b1_at | | | | | | | | |
| 15 | AFFX- | K00131 mouse b2 repeat sequence from clone mm61 | | | | | | | |
| | MURINE_{—} | | | | | | | | |
| | B2_at | | | | | | | | |
| 15 | 92541_at | Cluster Incl X12973 Murine MLC1F/MLC3F gene for myosin alkali light chain (exon 1) (last skeletal muscle isoform)/ | P | 2905.9 | P | 5018.7 | P | 4742.3 | P |
| | | cds = (125,238)/gb = X12973/gi = 53140/ug = Mm.1000/len = 250/STRA = for | | | | | | | |
| 15 | 92543_at | Cluster Incl AW123542 UI-M-BH2.1-aqb-f-01-0-UI.s1 Mus musculus cDNA, 3 end/clone = UI-M-BH2.1-aqb-f-01-0-UI/ | P | 205.6 | P | 327.7 | P | 227.5 | P |
| | | clone_end = 3/gb = AW123542/gi = 6099072/ug = Mm 100400/len = 373/STRA = for | | | | | | | |
| 15 | 92577_f_at | Cluster Incl c UI-M-BH1-alu-a-05-0-UI s1 Mus musculus cDNA, 3 end/clone = UI-M-BH1-alu-a-05-0-UI/clone_end = 3/ | P | 3629.7 | P | 6961.3 | P | 6079.7 | P |
| | | gb = AW047116/gi = 5907645/ug = Mm 10474/len = 309/STRA = for | | | | | | | |
| 15 | 92624_r_at | Cluster Incl U09189: Mus musculus Ioricrin gene, complete cds/cds = (23,1468)/gb = U09189/gi = 520479/ug = Mm 1121/ | P | 2198.8 | P | 4148.8 | P | 3833.2 | P |
| | | len = 1704/STRA = for | | | | | | | |
| 15 | 92628_at | Cluster Incl X75895 M musculus mRNA for ribosomal protein L36/cds = (20,337)/gb = X75895/gi = 440801/ug = Mm 11376/ | P | 4381 | P | 6987.8 | P | 6415.2 | P |
| | | len = 387/STRA = for | | | | | | | |
| 15 | 92629_f_at | Cluster Incl D63707 Mouse mRNA for hepatoma derived growth factor (HDGF), complete cds/cds = (61,774)/gb = D63707/ | P | 2697.3 | P | 3582.5 | P | 3308.1 | P |
| | | gi = 945418/ug = Mm 1141/len = 1563/STRA = for | | | | | | | |
| 15 | 92631_f_at | Cluster Incl M19380 Calmodulin 3/cds = (109,558)/gb = M19380/gi = 469419/ug = Mm 1147/len = 722/STRA = for | P | 496.7 | P | 1008.7 | P | 757 | P |
| 15 | 92783_at | Cluster Incl X02891 Growth hormone/cds = (61,711)/gb = X02891/gi = 51067/ug = Mm.1240/len = 777/STRA = for | P | 738.1 | P | 1119.7 | P | 769.5 | P |
| 15 | 92880_at | Cluster Incl M38337 Milk fat globule-EGF factor 8 protein/cds = (45,1436)/gb = M38337/gi = 199142/ug = Mm 1451/len = 2077/ | P | 1588.3 | P | 1797.3 | P | 1826.1 | P |
| | | STRA = for | | | | | | | |
| 15 | 92881_at | Cluster Incl U77943 Mus musculus myosin light chain 2 mRNA, complete cds/cds = (0,509)/gb = U77943/gi = 1675395/ | P | 1607.1 | P | 3006.4 | P | 2491.2 | P |
| | | ug = Mm 14526/len = 580/STRA = for | | | | | | | |
| 15 | 93030_at | Cluster Incl AI844457: UI-M-AJ1-ahe-c-02-0-UI s1 Mus musculus cDNA, 3 end/clone = UI-M-AJ1-ahe-c-02-0-UI/ | P | 3151.2 | P | 4866.5 | P | 4804 | P |
| | | clone_end = 3/gb = AI844457/gi = 5488363/ug = Mm 14833/len = 347/STRA = for | | | | | | | |
| 15 | 93038_f_at | Cluster Incl M69260: Lipocortin 1/cds = (14,1054)/gb = M69260/gi = 198844/ug = Mm.14860/len = 1340/STRA = for | P | 3388.1 | P | 4355.3 | P | 4122.8 | P |
| 15 | 93076_at | Cluster Incl AW124171: UI-M-BH2 1-apr-h-07-0-UI s1 Mus musculus cDNA, 3 end/clone = UI-M-BH2 1-apr-h-07-0-UI/ | P | 2446.1 | P | 2883.1 | P | 2621 | P |
| | | clone_end = 3/gb = AW124171/gi = 6099701/ug = Mm 1581/len = 173/STRA = for | | | | | | | |
| 15 | 93077_s_at | Cluster Incl D86232 Lymphocyte antigen 6 complex, locus C/cds = (144,452)/gb = D86232/gi = 1695689/ug = Mm 1583/ | P | 1843.7 | P | 2578.3 | P | 2416.6 | P |
| | | len = 2683/STRA = for | | | | | | | |
| 15 | 93101_s_at | Cluster Incl U96635 Neural precursor cell expressed, developmentally down-regulated gene 4/cds = (611,2944)/gb = U96635/ | P | 1480.4 | P | 2261.1 | P | 2020.4 | P |
| | | gi = 2088622/ug = Mm 16553/len = 5581/STRA = for | | | | | | | |
| 15 | 93116_at | Cluster Incl J02626 Protein kinase, cAMP dependent, catalytic, beta/cds = (192,1247)/gb = J02826/gi = 200386/ | P | 895.2 | P | 1404.6 | P | 1253 | P |
| | | ug = Mm 16766/len = 1272/STRA = for | | | | | | | |
| 15 | 93271_s_at | Cluster Incl AJ245401: Mus musculus Nesp gene for neuroendocrine secretory peptide 55/cds = (0,761)/gb = AJ245401/ | P | 2707.9 | P | 5234.7 | P | 4434.6 | P |
| | | gi = 6010255/ug = Mm 17446/len = 762/STRA = for | | | | | | | |
| 15 | 93324_at | Cluster Incl M58566 Mouse TIS11 primary response gene, complete cds/cds = (67,1083)/gb = M58566/gi = 202064/ | P | 1991.4 | P | 3276.8 | P | 2869.8 | P |
| | | ug = Mm 18571/len = 2659/STRA = for | | | | | | | |
| 15 | 93534_at | Cluster Incl X53929 Decorin/cds = (231,1295)/gb = X53929/gi = 53668/ug = Mm 1987/len = 1372/STRA = for | P | 3329.4 | P | 6112.4 | P | 5761.1 | P |
| 15 | 93535_at | Cluster Incl AF041472: Mus musculus ataxin-2 (SCA2) mRNA, complete cds/cds = (26,3883)/gb = AF041472/gi = 3005019/ | P | 422.6 | P | 663.8 | P | 662.6 | P |
| | | ug = Mm 1989/len = 4225/STRA = for | | | | | | | |
| 15 | 93543_f_at | Cluster Incl J03952 Mouse, glutathione transferase GT8 7 mRNA, complete cds/cds = (12,668)/gb = J03952/gi = 193687/ | P | 921.4 | P | 1734.6 | P | 1500.6 | P |
| | | ug = Mm 2011/len = 1018/STRA = for | | | | | | | |
| 15 | 93604_f_at | Cluster Incl AF061260 Mus musculus immunosuperfamily protein BI2 mRNA, complete cds/cds = (308,1195)/ | P | 1250 | P | 1730.4 | P | 1928.5 | P |
| | | gb = AF061260/gi = 3779241/ug = Mm 20921/len = 1861/STRA = for | | | | | | | |
| 15 | 93730_at | Cluster Incl AF085809 Mus musculus synapsin lb (Synl) mRNA, complete cds/cds = (135,2147)/gb = AF085809/gi = 3599472/ | P | 2037.5 | P | 4084 | P | 3586.7 | P |
| | | ug = Mm 20936/len = 3694/STRA = for | | | | | | | |
| 15 | 93740_at | Cluster Incl U33196 Mus musculus Y-box binding protein mYB-1a mRNA, complete cds/cds = (115,1083)/gb = U33196/ | P | 2825.8 | P | 5036.9 | P | 4838.8 | P |
| | | gi = 988280/ug = Mm 21054/len = 1194/STRA = for | | | | | | | |
| 15 | 93744_at | Cluster Incl AI119347 ue98e05.y1 Mus musculus cDNA, 5 end/clone = IMAGE-1499168/clone_end = 5/gb = AI119347/ | P | 1668.2 | P | 4283.1 | P | 2786 | P |
| | | gi = 3519671/ug = Mm 21075/len = 476/STRA = for | | | | | | | |
| 15 | 93748_at | Cluster Incl AW123901.UI-M-BH2 1-app-g-06-0-UI.s1 Mus musculus cDNA, 3 end/clone = UI-M-BH2 1-app-g-06-0-UI/ | P | 1248.3 | P | 1609 | P | 1525.4 | P |
| | | clone_end = 3/gb = AW123901/gi = 6099431/ug = Mm 21094/len = 226/STRA = for | | | | | | | |
| 15 | 93762_at | Cluster Incl AI841493 UI-M-AH0-acy-d-06-0-UI s1 Mus musculus cDNA, 3 end/clone = UI-M-AH0-acy-d-06-0-UI/ | P | 1157.7 | P | 1610.1 | P | 1394.8 | P |
| | | clone_end = 3/gb = AI841493/gi = 5475706/ug = Mm 21158/len = 367/STRA = for | | | | | | | |
| 15 | 93991_at | Cluster Incl X07295 Malate dehydrogenase, mitochondrial/cds = (0,1016)/gb = X07295/gi = 50679/ug = Mm.21743/len = 1017/ | P | 3177.4 | P | 4637 | P | 4207.8 | P |
| | | STRA = for | | | | | | | |
| 15 | 94068_at | Cluster Incl AW048899 UI-M-BH1-amf-h-03-0-UI s1 Mus musculus cDNA, 3 end/clone = UI-M-BH1-amf-h-03-0-UI/ | P | 2189.6 | P | 3692.6 | P | 3209.3 | P |
| | | clone_end = 3/gb = AW048899/gi = 5909428/ug = Mm 22103/len = 481/STRA = for | | | | | | | |
| 15 | 94247_at | Cluster Incl AA600542: vm75a03 r1 Mus musculus cDNA, 5 end/clone = IMAGE-1004044/clone_end = 5/gb = AA600542/ | P | 2982.6 | P | 5809.8 | P | 5420.4 | P |
| | | gi = 2434167/ug = Mm 22365/len = 140/STRA = for | | | | | | | |
| 15 | 94305_at | Cluster Incl U03419: Mus musculus alpha-1 type I procollagen mRNA, partial cds/cds = (0,977)/gb = U03419/gi = 424103/ | P | 2889.2 | P | 4682 | P | 4042.9 | P |
| | | ug = Mm.22621/len = 1205/STRA = for | | | | | | | |
| 15 | 94321_at | Cluster Incl V00830 Keratin complex 1, acidic, gene 10/cds = (27,1739)/gb = V00830/gi = 52786/ug = Mm.22662/len = 1944/ | P | 2463.1 | P | 4459.1 | P | 4457.3 | P |
| | | STRA = for | | | | | | | |
| 15 | 94823_at | Cluster Incl AI835315 UI-M-AQ0-aah-a-06-0-UI s1 Mus musculus cDNA, 3 end/clone = UI-M-AQ0-aah-a-06-0-UI/ | P | 3457.3 | P | 5969.7 | P | 5758.7 | P |
| | | clone_end = 3/gb = AI835315/gi = 5469528/ug = Mm 22723/len = 275/STRA = for | | | | | | | |
| 15 | 94839_at | Cluster Incl M96823: Mouse nucleobindin mRNA, complete cds/cds = (18,1385)/gb = M96823/gi = 200113/ug = Mm.2283/ | P | 509.5 | P | 778.3 | P | 600 | P |
| | | len = 2116/STRA = for | | | | | | | |
| 15 | 94845_at | Cluster Incl AW125736: UI-M-BH2.2-aqi-g-02-0-U1.s1 Mus musculus cDNA, 3 end/clone = UI-M-BH2.2-aqi-g-02-0-Ul/ | P | 1114.3 | P | 1185.3 | P | 1341.8 | P |
| | | clone_end = 3/gb = AW125736/gi = 6101231/ug = Mm.23014/len = 426/STRA = for | | | | | | | |
| 15 | 95435_at | Cluster Incl AI844009 UI-M-AL1-ahi-a-05-0-UI s1 Mus musculus cDNA, 3 end/clone = UI-M-AL1-ahi-a-05-0-UI/ | P | 236.7 | P | 311.9 | P | 320.8 | P |
| | | clone_end = 3/gb = AI844009/gi = 5487910/ug = Mm 24499/len = 408/STRA = for | | | | | | | |
| 15 | 95440_at | Cluster Incl AI846093 UI-M-AK1-aew-c-02-0-UI.s1 Mus musculus cDNA, 3 end/clone = UI-M-AK1-aew-c-02-0-UI/ | P | 126.3 | P | 214.8 | P | 192.1 | P |
| | | clone_end = 3/gb = AI846093/gi = 5489964/ug = Mm 24564/len = 284/STRA = for | | | | | | | |
| 15 | 95458_s_at | Cluster Incl AW121960 UI-M-BH2 3-aoi-h-09-0-UI s1 Mus musculus cDNA, 3 end/clone = UI-M-BH2 3-aoi-h-09-0-UI/ | P | 1090 | P | 1982.4 | P | 1669.3 | P |
| | | clone_end = 3/gb = AW121960/gi = 6097311/ug = Mm 24703/len = 370/STRA = for | | | | | | | |
| 15 | 95490_at | Cluster Incl AW120891: UI-M-BH2 3-aob-b-02-0-UI s1 Mus musculus cDNA, 3 end/clone = UI-M-BH2.3-aob-b-02-0-UI/ | P | 1415.2 | P | 1801.1 | P | 1574.6 | P |
| | | clone_end = 3/gb = AW120891/gi = 6096224/ug = Mm 25045/len = 178/STRA = for | | | | | | | |
| 15 | 96018_r_at | Cluster Incl AI853561 UI-M-BH0-ajd-h-09-0-UI s1 Mus musculus cDNA, 3 end/clone = UI-M-BH0-ajd-h-09-0-UI/ | P | 273 | P | 386.7 | P | 436.9 | P |
| | | clone_end = 3/gb = AI853561/gi = 5497467/ug = Mm 25634/len = 480/STRA = for | | | | | | | |
| 15 | 96066_s_at | Cluster Incl X97047: Pyruvate kinase 3/cds = (6,1601)/gb = X97047/gi = 1405932/ug = Mm.2635/len = 2134/STRA = for | P | 2537.7 | P | 3803.3 | H | 3752.2 | P |
| 15 | 96072_at | Cluster Incl M17516: Mouse lactate dehydrogenase A-4 (LDH-A) mRNA, complete cds/cds = (0,395)/gb = M17516/ | P | 3175.9 | P | 4889.9 | P | 4015.1 | P |
| | | gi = 198776/ug = Mm.26504/len = 881/STRA = for | | | | | | | |
| 15 | 96699_at | Cluster Incl X53476 High mobility group protein 14/cds = (33,323)/gb = X53476/gi = 51334/ug = Mm.2756/len = 1070/ | P | 3961.4 | P | 6950.3 | P | 5838.3 | P |
| | | STRA = for | | | | | | | |
| 15 | 97229_at | Cluster Incl AW061042: UI-M-BH1-akk-b-09-0-Ul s2 Mus musculus cDNA, 3 end/clone = UI-M-BH1-akk-b-09-0-UI/ | P | 1439.9 | P | 2110.6 | P | 1917.4 | P |
| | | clone_end = 3/gb = AW061042/gi = 6008793/ug = Mm 27796/len = 502/STRA = for | | | | | | | |
| 15 | 97808_at | Cluster Incl AI844532 UI-M-AJ1-ahf-d-05-0-UI.s2 Mus musculus cDNA, 3 end/clone = UI-M-AJ1-ahf-d-05-0-UI/ | P | 719.3 | P | 1562.3 | P | 1364.6 | P |
| | | clone_end = 3/gb = AI844532/gi = 5488438/ug = Mm 28150/len = 521/STRA = for | | | | | | | |
| 15 | 97825_at | Cluster Incl AI854029 UI-M-BH0-ajm-c-04-0-UI s1 Mus musculus cDNA, 3 end/clone = UI-M-BH0-ajm-c-04-0-UI/ | P | 3953.1 | P | 6445.9 | P | 6070.8 | P |
| | | clone_end = 3/gb = AI854029/gi = 5497935/ug = Mm 28209/len = 528/STRA = for | | | | | | | |
| 15 | 98922_at | Cluster Incl L34260 Integral membrane protein 1/cds = (111,2228)/gb = L34260/gi = 508542/ug = Mm 2863/len = 3094/ | P | 68.7 | P | 172.4 | P | 157.2 | P |
| | | STRA = for | | | | | | | |
| 15 | 98927_at | Cluster Incl AI851048: UI-M-BH0-ajv-f-08-0-UI s1 Mus musculus cDNA, 3 end/clone = UI-M-BH0-ajv-f-08-0-UI/ | P | 937.7 | P | 1653.6 | P | 1467.8 | P |
| | | clone_end = 3/gb = AI851048/gi = 5494954/ug = Mm 28650/len = 129/STRA = for | | | | | | | |
| 15 | 94464_at | Cluster Incl AF029347 Mus musculus chloride channel protein 3 (CLCN3) mRNA, complete cds/cds = (0,2456)/ | P | 163.8 | P | 365.2 | P | 278.1 | P |
| | | gb = AF029347/gi = 2599549/ug = Mm.28842/len = 2457/STRA = for | | | | | | | |
| 15 | 94481_at | Cluster Incl AI851321 UI-M-BH0-akh-a-06-0-UI s1 Mus musculus cDNA, 3 end/clone = UI-M-BH0-akh-a-06-0-UI/ | P | 381.5 | P | 523.3 | P | 594.7 | P |
| | | clone_end = 3/gb = AI851321/gi = 5495227/ug = Mm 28877/len = 420/STRA = for | | | | | | | |
| 15 | 94536_s_at | Cluster Incl AI843417: UI-M-AQ1-aec-a-10-0-UI s1 Mus musculus cDNA, 3 end/clone = UI-M-AQ1-aec-a-10-0-UI/ | P | 2654 | P | 3414.8 | P | 3547.9 | P |
| | | clone_end = 3/gb = AI843417/gi = 5477630/ug = Mm 29057/len = 345/STRA = for | | | | | | | |
| 15 | 95046_s_at | Cluster Incl M98036 Mus musculus guanine nucleotide exchange factor delta subunit (JGR1A) mRNA, complete cds/ | P | 386.2 | P | 674.6 | P | 548.8 | P |
| | | cds = (69,1643)/gb = M98036/gi = 529429/ug = Mm 29128/len = 1677/STRA = for | | | | | | | |
| 15 | 95064_at | Cluster Incl AI849271 UI-M-AJ1-agz-g-06-0-UI s1 Mus musculus cDNA, 3 end/clone = UI-M-AJ1-agz-g-06-0-UI/ | P | 3022.6 | P | 4931.3 | P | 4624.4 | P |
| | | clone_end = 3/gb = AI849271/gi = 5493177/ug = Mm 29165/len = 405/STRA = rev | | | | | | | |
| 15 | 95072_at | Cluster Incl AW121892 UI-M-BH2.3-aoi-e-12-0-UI s1 Mus musculus cDNA, 3 end/clone = UI-M-BH2 3-aoi-e-12-0-UI/ | P | 2754.8 | P | 3763 | P | 3149.3 | P |
| | | clone_end = 3/gb = AW121892/gi = 6097266/ug = Mm 29196/len = 438/STRA = for | | | | | | | |
| 15 | 95124_i_at | Cluster Incl AW122337: UI-M-BH2.2-aos-g-04-0-UI s1 Mus musculus cDNA, 3 end/clone = UI-M-BH2 2-aos-g-04-0-UI/ | P | 858.6 | P | 1428.2 | P | 965.1 | P |
| | | clone_end = 3/gb = AW122337/gi = 6097822/ug = Mm 29405/len = 403/STRA = for | | | | | | | |
| 15 | 95125_f_at | Cluster Incl AW122337: UI-M-BH2 2-aos-g-04-0-UI s1 Mus musculus cDNA, 3 end/clone = UI-M-BH2 2-aos-g-04-0-UI/ | P | 1501.3 | P | 2489.1 | P | 2113.7 | P |
| | | clone_end = 3/gb = AW122337/gi = 6097822/ug = Mm 29405/len = 403/STRA = for | | | | | | | |
| 15 | 95132_r_at | Cluster Incl AI852592 UI-M-BH0-aiu-b-11-0-UI s1 Mus musculus cDNA, 3 end/clone = UI-M-BH0-aiu-b-11-0-UI/ | P | 1330.3 | P | 1854 | P | 1790.3 | P |
| | | clone_end = 3/gb = AI852592/gi = 5496498/ug = Mm 29415/len = 392/STRA = rev | | | | | | | |
| 15 | 95661_at | Cluster Incl L08115: CD9 antigen/cds = (54,734)/gb = L08115/gi = 388911/ug = Mm 2956/len = 1130/STRA = for | P | 2715.4 | P | 3552.9 | P | 3292.9 | P |
| 15 | 95698_at | Cluster Incl AI851810: UI-M-BH0-ais-c-02-0-UI s1 Mus musculus cDNA, 3 end/clone = UI-M-BH0-ais-c-02-0-UI/ | P | 1195.2 | P | 2199.7 | P | 2141.4 | P |
| | | clone_end = 3/gb = AI851810/gi = 5495716/ug = Mm 29683/len = 276/STRA = for | | | | | | | |
| 15 | 95705_s_at | Cluster Incl J04181: Mouse A-X actin mRNA complete cds/cds = (62,1189)/gb = J04181/gi = 191660/ug = Mm 297/len = 1857/ | P | 2875.9 | P | 4826.8 | P | 4763.9 | P |
| | | STRA = for | | | | | | | |
| 15 | 95706_at | Cluster Incl X16834 Mouse mRNA for Mac-2 antigen/cds = (43,837)/gb = X16834/gi = 52986/ug = Mm 2970/len = 955/ | P | 2223.7 | P | 3322.6 | P | 2713.1 | P |
| | | STRA = for | | | | | | | |
| 15 | 95713_at | Cluster Incl D67067 Mouse mRNA for thymic epithelial cell surface antigen, complete cds/cds = (12,1559)/gb = D67067/ | P | 1333.3 | P | 1676.4 | P | 1896.2 | P |
| | | gi = 2059325/ug = Mm 29710/len = 2446/STRA = for | | | | | | | |
| 15 | 95723_r_at | Cluster Incl AI841464: UI-M-AH0-acy-a-04-0-UI.s1 Mus musculus cDNA, 3 end/clone = UI-M-AH0-acy-a-04-0-UI/ | P | 1940.2 | P | 3525.5 | P | 3106 | P |
| | | clone_end = 3/gb = AI841464/gi = 5475677/ug = Mm.29731/len = 413/STRA = for | | | | | | | |
| 15 | 95740_at | Cluster Incl AA727291: vu95a10 r1 Mus musculus cDNA, 5 end/clone = IMAGE-1209882/clone_end = 5/gb = AA727291/ | P | 2913.2 | P | 4992.9 | P | 5037.6 | P |
| | | gi = 2744998/ug = Mm.29753/len = 279/STRA = for | | | | | | | |
| 15 | 95755_at | Cluster Incl D14485: Mouse mRNA for dbpA murine homologue, complete cds/cds = (183,1109)/gb = D14485/gi = 1160330/ | P | 2217.9 | P | 3418.7 | P | 3423.3 | P |
| | | ug = Mm 29794/len = 1647/STRA = for | | | | | | | |
| 15 | 96290_f_at | Cluster Incl U93863: Mus musculus ribosomal protein L21 mRNA, complete cds/cds = (36,518)/gb = U93863/gi = 1938403/ | P | 3977 | P | 7041.8 | P | 6496.3 | P |
| | | ug = Mm 29896/len = 612/STRA = for | | | | | | | |
| 15 | 96298_f_at | Cluster Incl AF020185: Mus musculus protein inhibitor of nitric oxide synthase (PIN) mRNA, complete cds/cds = (99,388)/ | P | 3699.6 | P | 7342.3 | P | 6069.4 | P |
| | | gb = AF020185/gi = 4103058/ug = Mm.29908/len = 531/STRA = for | | | | | | | |
| 15 | 96300_f_at | Cluster Incl AI8542318 UI-M-BH0-ajz-g-10-0-UI s1 Mus musculus cDNA, 3 end/clone = UI-M-BH0-ajz-g-10-0-UI/ | P | 2621 | P | 4771.7 | P | 4567.3 | P |
| | | clone_end = 3/gb = AI854238/gi = 5498144/ug = Mm.29911/len = 349/STRA = for | | | | | | | |
| 15 | 96307_s_at | Cluster Incl AW121947 UI-M-BH2 3-aoj-f-05-0-UI s1 Mus musculus cDNA, 3 end/clone = UI-M-BH2 3-aoj-f-05-0-UI/ | P | 1709.6 | P | 4045.5 | P | 3281.1 | P |
| | | clone_end = 3/gb = AW121947/gi = 6097393/ug = Mm.29919/len = 347/STRA = for | | | | | | | |
| 15 | 96327_at | Cluster Incl AI852535: UI-M-BH0-aiw-e-05-0-UI.s1 Mus musculus cDNA, 3 end/clone = UI-M-BH0-aiw-e-05-0-UI/ | P | 593.8 | P | 650.6 | P | 751.1 | P |
| | | clone_end = 3/gb = AI852535/gi = 5496441/ug = Mm 29962/len = 357/STRA = for | | | | | | | |
| 15 | 96358_at | Cluster Incl AI837403: UI-M-AP0-abm-e-02-0-UI.s1 Mus musculus cDNA, 3 end/clone = UI-M-AP0-abm-e-02-0-UI/ | P | 1297.9 | P | 3887.3 | P | 2631.1 | P |
| | | clone_end = 3/gb = AI837403/gi = 5471616/ug = Mm.30011/len = 404/STRA = for | | | | | | | |
| 15 | 96859_at | Cluster Incl AB026621: Mus musculus mRNA for RIE2. complete cds/cds = (481,2895)/gb = AB026621/gi = 5931597/ | P | 1338.7 | P | 2586.5 | P | 2065.5 | P |
| | | ug = Mm 30051/len = 3110/STRA = for | | | | | | | |
| 15 | 96872_at | Cluster Incl AI835846: UI-M-AI0-aat-b-06-0-UI s1 Mus musculus cDNA, 3 end/clone = UI-M-AI0-aat-b-06-0-UI/clone_end = 3/ | P | 1152.6 | P | 1449.1 | P | 1621 | P |
| | | gb = AI835846/gi = 5470059/ug = Mm.30068/len = 182/STRA = for | | | | | | | |
| 15 | 96942_at | Cluster Incl AI844011: UI-M-AL1-ahi-b-01-0-UI s1 Mus musculus cDNA, 3 end/clone = UI-M-AL1-ahi-b-01-0-UI/ | P | 2254 | P | 3357.8 | P | 3199.8 | P |
| | | clone_end = 3/gb = AI844011/gi = 5487917/ug = Mm 30194/len = 338/STRA = for | | | | | | | |
| 15 | 96949_at | Cluster Incl AI853542 UI-M-BH0-ajd-g-01-0-UI s1 Mus musculus cDNA, 3 end/clone = UI-M-BH0-ajd-g-01-0-UI/ | P | 641.4 | P | 1082.9 | P | 748.2 | P |
| | | clone_end = 3/gb = AI853542/gi = 5497448/ug = Mm 30205/len = 110/STRA = for | | | | | | | |
| 15 | 97460_at | Cluster Incl AW122823: UI-M-BH2 1-aoz-c-09-0-UI.s1 Mus musculus cDNA, 3 end/clone = UI-M-BH2 1-aoz-c-09-0-UI/ | P | 1548.4 | P | 2053.3 | P | 1892.6 | P |
| | | clone_end = 3/gb = AW122823/gi = 6098353/ug = Mm 30272/len = 219/STRA = for | | | | | | | |
| 15 | 97484_at | Cluster Incl AI852704: UI-M-BH0-aji-e-07-0-UI s1 Mus musculus cDNA, 3 end/clone = UI-M-BH0-aji-e-07-0-UI/clone_end = 3/ | P | 917.6 | P | 1191.9 | P | 923.2 | P |
| | | gb = AI852704/gi = 5496610/ug = Mm.30826/len = 427/STRA = for | | | | | | | |
| 15 | 97544_at | Cluster Incl D83037 Mouse mRNA for 14-3-3 zeta, complete cds/cds = (87,824)/gb = D83037/gi = 1181605/ug = Mm 3360/ | P | 1765.1 | P | 2116.8 | P | 2191 | P |
| | | len = 1935/STRA = for | | | | | | | |
| 15 | 97553_at | Cluster Incl AI841884 UI-M-AO0-acd-c-10-0-UI.s1 Mus musculus cDNA, 3 end/clone = UI-M-AO0-acd-c-10-0-UI/ | P | 428 | P | 543.8 | P | 498.6 | P |
| | | clone_end = 3/gb = AI841884/gi = 5476097/ug = Mm 33808/len = 370/STRA = for | | | | | | | |
| 15 | 98059_s_at | Cluster Incl D49733: Lamin A/cds = (0,1388)/gb = D49733/gi = 1838920/ug = Mm.3438/len = 1480/STRA = for | P | 3680.8 | P | 5746.1 | P | 5799.3 | P |
| 15 | 98078_at | Cluster Incl AI157911 ue57h08 r1 Mus musculus cDNA, 5 end/clone = IMAGE-1495263/clone_end = 5/gb = AI157911/ | P | 246.9 | P | 319.3 | P | 396.8 | P |
| | | gi = 3686380/ug = Mm 34556/len = 439/STRA = for | | | | | | | |
| 15 | 98094_f_at | Cluster Incl AI843627 UI-M-AO1-aen-f-03-0-UI.s1 Mus musculus cDNA, 3 end/clone = UI-M-AO1-aen-f-03-0-UI/ | P | 508 | P | 615.8 | P | 622.8 | P |
| | | clone_end = 3/gb = AI843627/gi = 5477840/ug = Mm.34641/len = 425/STRA = for | | | | | | | |
| 15 | 98119_at | Cluster Incl K02928 Ribosomal protein L30/cds = (70,417)/gb = K02928/gi = 435126/ug = Mm 3487/len = 468/STRA = for | P | 3526.5 | P | 6566 | P | 6006.1 | P |
| 15 | 98152_at | Cluster Incl AW122407 UI-M-BH2 2-aow-e-09-0-UI.s1 Mus musculus cDNA, 3 end/clone = UI-M-BH2.2-aow-e-09-0-UI/ | P | 1068.9 | P | 1734.9 | P | 1504.4 | P |
| | | clone_end = 3/gb = AW122407/gi = 6097909/ug = Mm 35738/len = 471/STRA = for | | | | | | | |
| 15 | 98154_at | Cluster Incl AW050133 UI-M-BH1-ano-d-11-0-UI s1 Mus musculus cDNA, 3 end/clone = UI-M-BH1-ano-d-11-0-UI/ | P | 305.6 | P | 405 | P | 510.4 | P |
| | | clone_end = 3/gb = AW050133/gi = 5910662/ug = Mm 35779/len = 521/STRA = for | | | | | | | |
| 15 | 98557_f_at | Cluster Incl U65636 Mus musculus beta proteasome subunit (Lmp3) mRNA, complete cds/cds = (5,799)/gb = U65636/ | P | 1989.7 | P | 2736.4 | P | 2444.5 | P |
| | | gi = 1762778/ug = Mm 368/len = 892/STRA = for | | | | | | | |
| 15 | 98564_f_at | Cluster Incl U67770 Mus musculus ribosomal protein S26 (RPS26) mRNA, complete cds/cds(5,352)/gb = U67770/ | P | 2238.2 | P | 4509.7 | P | 4135.4 | P |
| | | gi = 1527175/ug = Mm 372/len = 426/STRA = for | | | | | | | |
| 15 | 98571_s_at | Cluster Incl U48363 Nascent polypeptide-associated complex alpha polypeptide/cds = (51,698)/gb = U48363/gi = 1686688/ | P | 3392.5 | P | 5112 | P | 4667.8 | P |
| | | ug = Mm 3746/len = 829/STRA = for | | | | | | | |
| 15 | 99093_at | Cluster Incl AI852864: UI-M-BH0-ajk-e-04-0-UI s1 Mus musculus cDNA, 3 end/clone = UI-M-BH0-ajk-e-04-0-UI/ | P | 4012 | P | 7032.5 | P | 6042 | P |
| | | clone_end = 3/gb = AI852864/gi = 5496770/ug = Mm 39130/len = 308/STRA = for | | | | | | | |
| 15 | 99096_at | Cluster Incl U46690 Mus musculus ATP-dependent RNA helicase mRNA, partial cds/cds = (0,1441)/gb = U46690/ | P | 213.4 | P | 305.7 | P | 321.4 | P |
| | | gi = 1262844/ug = Mm 3935/len = 1674/STRA = for | | | | | | | |
| 15 | 99120_f_at | Cluster Incl R75450 MDB0625 Mus musculus cDNA, 3 end/clone_end = 3/gb = R75450/gi = 849694/ug = Mm.4024/ | P | 1950.5 | P | 2938.3 | P | 2398.7 | P |
| | | len = 329/STRA = for | | | | | | | |
| 15 | 99126_at | Cluster Incl L04961 Inactive X specific transcripts/cds = UNKNOWN/gb = L04961/gi = 202420/ug = Mm 4095/len = 14739/ | P | 1842.4 | P | 5306.5 | P | 5017 | P |
| | | STRA = for | | | | | | | |
| 15 | 99135_at | Cluster Incl U43076 Mus musculus cdc37 homolog mRNA, complele cds/cds = (87,1226)/gb = U43078/gi = 1375488/ | P | 702.5 | P | 1299.2 | P | 1174.4 | P |
| | | ug = Mm 4117/len = 1607/STRA = for | | | | | | | |
| 15 | 99586_at | Cluster Incl U10098 Cystalin 3/cds = (0,422)/gb = U10098/gi = 497414/ug = Mm 4263/len = 423/STRA = for | P | 3226.4 | P | 4761.8 | P | 5003.8 | P |
| 15 | 99590_at | Cluster Incl D25213 Mouse rpS17 mRNA for ribosomal protein S17, complete cds/cds = (11,418)/gb = D25213/gi = 893394/ | P | 2577.5 | P | 4780.5 | P | 4104.8 | P |
| | | ug = Mm 42767/len = 453/STRA = for | | | | | | | |
| 15 | 99592_f_at | Cluster Incl AB030505 Mus musculus mRNA for UBE-1c1, UBE-1c2, UBE-1c3, complete cds/cds = UNKNOWN/ | P | 886.8 | P | 1303.1 | P | 1090.8 | P |
| | | gb = AB030505/gi = 5668736/ug = Mm.42795/len = 4190/STRA = for | | | | | | | |
| 15 | 99640_at | Cluster Incl AW045481: UI-M-BH1-ahr-c-09-0-UI s1 Mus musculus cDNA, 3 end/clone = UI-M-BH1-ai.r-c-09-0-UI/ | P | 567 | P | 803.6 | P | 830.5 | P |
| | | clone_end = 3/gb = AW045481/gi = 5905975/ug = Mm 43580/len = 398/STRA = for | | | | | | | |
| 15 | 99660_f_at | Cluster Incl X52940: Cytochrome C oxidase subunit VIIc/cds = (86,277)/gb = X52940/gi = 50524/ug = Mm 43786/len = 403/ | P | 2243.5 | P | 5944.8 | P | 5388.1 | P |
| | | STRA = for | | | | | | | |
| 15 | 99661_r_at | Cluster Incl X52940: Cytochrome C oxidase subunit VIIc/cds = (86,277)/gb = X52940/gi = 50524/ug = Mm.43786/len = 403/ | P | 856 | P | 2334.7 | P | 2340.7 | P |
| | | STRA = for | | | | | | | |
| 15 | 99669_at | Cluster Incl X15986: Mouse 3 mRNA for beta-galactoside specific lectin (14 kDa)/cds = (43,450)/gb = X15986/gi = 50162/ | P | 3975.8 | P | 6147.8 | P | 5794.6 | P |
| | | ug = Mm 43831/len = 495/STRA = for | | | | | | | |
| 15 | 100043_f_at | Cluster Incl AI173973: ui43c05 x1 Mus musculus cDNA, 3 end/clone = IMAGE-1885160/clone_end = 3/gb = AI173973/ | P | 2491.6 | P | 4138.5 | P | 4048.8 | P |
| | | gi = 3720115/ug = Mm 44242/len = 459/STRA = for | | | | | | | |
| 15 | 100064_f_at | Cluster Incl M63801 Mus musculus connexin 43 (alpha-1 gap junction) mRNA, complete cds/cds = (220,1388)/gb = M63801/ | P | 3230.4 | P | 5538.3 | P | 5233.8 | P |
| | | gi = 191773/ug = Mm 4504/len = 1408/STRA = for | | | | | | | |
| 15 | 100569_at | Cluster Incl M14044 Mouse calpactin I heavy chain (p36) mRNA, complete cds/cds = (7,1026)/gb = M14044/gi = 192353/ | P | 2829.4 | P | 4753.6 | P | 4831.1 | P |
| | | ug = Mm 584/len = 1307/STRA = for | | | | | | | |
| 15 | 100618_f_at | Cluster Incl AA062013 mj83h01 r1 Mus musculus cDNA, 5 end/clone = IMAGE-482737/clone_end = 5/gb = AA062013/ | P | 966.9 | P | 1828.5 | P | 1767.1 | P |
| | | gi = 1555829/ug = Mm.658/len = 529/STRA = for | | | | | | | |
| 15 | 101093_at | Cluster Incl M15832: Procollagen, type IV, alpha 1/cds = (0,689)/gb = M15832/gi = 192282/ug = Mm 738/len = 690/STRA = for | P | 3017.8 | P | 5114.9 | P | 4618.8 | P |
| 15 | 101113_at | Cluster Incl AI846668 UI-M-AN1-afi-c-10-0-UI s1 Mus musculus cDNA, 3 end/clone = UI-M-AN1-afi-c-10-0-UI/clone_end = 3/ | P | 1194.9 | P | 1996.2 | P | 1549.5 | P |
| | | gb = AI846668/gi = 5490574/ug = Mm.757/len = 262/STRA = for | | | | | | | |
| 15 | 101542_f_at | Cluster Incl L25126 D-E-A-D (aspartate-glutamahe-alanine-aspartate) box polypeptide 3/cds = (23,2011)/gb = L25126/ | P | 2021.8 | P | 3015.6 | P | 2732.2 | P |
| | | gi = 407995/ug = Mm 88188/len = 2187/STRA = for | | | | | | | |
| 15 | 101543_f_at | Cluster Incl M13441: Mouse alpha-tubulin isotype M-alpha-6 mRNA, complete cds/cds = (43,1392)/gb = M13441/gi = 202216/ | P | 1981.5 | P | 4030 | P | 3848.9 | P |
| | | ug = Mm.88212/len = 1495/STRA = for | | | | | | | |
| 15 | 101573_f_at | Cluster Incl AB020237: Mus musculus gene for ribosomal protein L27A, complete cds/cds = (0,446)/gb = AB020237/ | P | 3398.5 | P | 5951.7 | P | 5492.5 | P |
| | | gi = 4760603/ug = Mm 89853/len = 447/STRA = for | | | | | | | |
| 15 | 101577_at | Cluster Incl Z54209 M. musculus rpS6 gene/cds = (41,790)/gb = Z54209/gi = 1177548/ug = Mm.89902/len = 831/STRA = for | P | 2918.9 | P | 5272.9 | P | 4910.4 | P |
| 15 | 101578_f_at | Cluster Incl M12481 Actin, beta, cytoplasmic/cds = (0,1049)/gb = M12481/gi = 191581/ug = Mm.89919/len = 1109/STRA = for | P | 3175.2 | P | 5422.8 | P | 4669.4 | P |
| 15 | 102000_f_at | Cluster Incl AI842835 UI-M-AN1-afb-a-07-0-UI s1 Mus musculus cDNA, 3 end/clone = UI-M-AN1-afb-a-07-0-UI/ | P | 1005.9 | P | 1354 | P | 1101.8 | P |
| | | clone_end = 3/gb = AI842835/gi = 5477048/ug = Mm 988/len = 287/STRA = for | | | | | | | |
| 15 | 102028_at | Cluster Incl AF053959 Mus musculus putative ras effector Nore1 mRNA, complete cds/cds = (30,1271)/gb = AF053959/ | P | 485.8 | P | 566.6 | P | 600.5 | P |
| | | gi = 2997697/ug = Mm.10133/len = 3018/STRA = for | | | | | | | |
| 15 | 102364_at | Cluster Incl J04509 Jun proto-oncogene related gene d1/cds = (121,1146)/gb = J04509/gi = 198486/ug = Mm.1175/len = 1147/ | P | 2363 | P | 3836.9 | P | 3716.5 | P |
| | | STRA = for | | | | | | | |
| 15 | 102414_i_at | Cluster Incl U28423 Mus musculus protein kinase inhibitor p58 mRNA, complete cds/cds = (100,1614)/gb = U28423/ | P | 268.7 | P | 586.9 | P | 376.5 | P |
| | | gi = 1353271/ug = Mm.12616/len = 1615/STRA = for | | | | | | | |
| 15 | 102856_at | Cluster Incl AF047389 SRY-box containing gene 10/cds = (53,931)/gb = AF047389/gi = 3264587/ug = Mm 1493/len = 1463/ | P | 422.4 | P | 549.9 | P | 610.8 | P |
| | | STRA = for | | | | | | | |
| 15 | 102986_at | Cluster Incl M18779 Myogenic differentiation 1/cds = (159,1115)/gb = M18779/gi = 199994/ug = Mm 1526/len = 1785/ | P | 217.8 | P | 382.8 | P | 390.4 | P |
| | | STRA = for | | | | | | | |
| 15 | 103079_at | Cluster Incl AA982208: vw61e08 s1 Mus musculus cDNA, 3 end/clone = IMAGE-1248326/clone_end = 3/gb = AA982208/ | P | 743.6 | P | 1070.9 | P | 1141.7 | P |
| | | gi = 3160867/ug = Mm 17166/len = 415/STRA = for | | | | | | | |
| 15 | 103082_at | Cluster Incl AI847507: UI-M-AM1-afv-a-08-0-UI s1 Mus musculus cDNA, 3 end/clone = UI-M-AM1-afv-a-08-0-UI/ | P | 536 | P | 892.2 | P | 700.7 | P |
| | | clone_end = 3/gb = AI847507/gi = 5491413/ug = Mm 17173/len = 313/STRA = for | | | | | | | |
| 15 | 103388_at | Cluster Incl AW047050: UI-M-BH1-alr-c-07-0-UI.s1 Mus musculus cDNA, 3 end/clone = UI-M-BH1-alr-c-07-0-UI/ | P | 943.4 | P | 1307.8 | P | 1449.2 | P |
| | | clone_end = 3/gb = AW047050/gi = 5907579/ug = Mm 1865/len = 318/STRA = rev | | | | | | | |
| 15 | 103611_at | Cluster Incl AB012693 Mus musculus mRNA for CD47, complete cds/cds = (72,1046)/gb = AB012693/gi = 3036964/ | P | 863.8 | P | 1633.2 | P | 1606.3 | P |
| | | ug = Mm 20183/len = 1250/STRA = rev | | | | | | | |
| 15 | 103846_at | Cluster Incl AF038582 Mus musculus galectin-7 mRNA, complete cds/cds = (20,430)/gb = AF038562/gi = 2708653/ | P | 2411 | P | 4009.4 | P | 4083 | P |
| | | ug = Mm 20973/len = 588/STRA = for | | | | | | | |
| 15 | 103862_r_at | Cluster Incl AA388099: vc88g07 r1 Mus musculus cDNA, 5 end/clone = IMAGE-790140/clone_end = 5/gb = AA388099/ | P | 1059.2 | P | 1599.4 | P | 1545.4 | P |
| | | gi = 2041044/ug = Mm 21103/len = 564/STRA = for | | | | | | | |
| 15 | 103878_at | Cluster Incl AF103809 Mus musculus Ap-3 complex beta3A subunit (Ap3b1) mRNA, complete cds/cds = (63,3380)/ | P | 733.4 | P | 840.2 | P | 837.9 | P |
| | | gb = AF103809/gi = 3885987/ug = Mm 21185/len = 3938/STRA = for | | | | | | | |
| 15 | 103894_at | Cluster Incl AW060871: UI-M-BH1-amx-f-12-0-UI s1 Mus musculus cDNA, 3 end/clone = UI-M-BH1-amx-f-12-0-UI/ | P | 1882.1 | P | 2834.6 | P | 2749 | P |
| | | clone_end = 3/gb = AW060871/gi = 6008622/ug = Mm 21297/len = 463/STRA = for | | | | | | | |
| 15 | 104106_at | Cluster Incl AI837830: UI-M-AP0-abk-a-07-0-UI s1 Mus musculus cDNA, 3 end/clone = UI-M-AP0-abk-a-07-0-UI/ | P | 168.8 | P | 405.9 | P | 288.9 | P |
| | | clone_end = 3/gb = AI837830/gi = 5472043/ug = Mm 21897/len = 259/STRA = for | | | | | | | |
| 15 | 104311_at | Cluster Incl AW125241: UI-M-BH2 1-aps-e-11-0-UI s1 Mus musculus cDNA, 3 end/clone = UI-M-BH2 1-aps-e-11-0-UI/ | P | 219 | P | 370.8 | P | 327 | P |
| | | clone_end = 3/gb = AW125241/gi = 6100771/ug = Mm 22397/len = 320/STRA = for | | | | | | | |
| 15 | 104313_at | Cluster Incl AI842432 UI-M-AM1-afy-b-10-0-UI s1 Mus musculus cDNA, 3 end/clone = UI-M-AM1-afy-b-10-0-UI/ | P | 988.7 | P | 1626 | P | 1424.9 | P |
| | | clone_end = 3/gb = AI842432/gi = 5476645/ug = Mm 22403/len = 330/STRA = for | | | | | | | |
| 15 | 104341_at | Cluster Incl AW121406 UI-M-BH2 2-aom-h-09-0-UI s1 Mus musculus cDNA, 3 end/clone = UI-M-BH2 2-aom-h-09-0-UI/ | P | 872.2 | P | 1509 | P | 1617.9 | P |
| | | clone_end = 3/gb = AW121406/gi = 6096739/ug = Mm 22525/len = 498/STRA = for | | | | | | | |
| 15 | 104362_at | Cluster Incl AI853532 UI-M-BHO-ajd-f-02-0-UI s1 Mus musculus cDNA, 3 end/clone = UI-M-BH0-ajd-f-02-0-UI/ | P | 415.5 | P | 732.4 | P | 642.5 | P |
| | | clone_end = 3/gb = AI853532/gi = 5497438/ug = Mm.22599/len = 353/STRA = for | | | | | | | |
| 15 | 104391_s_at | Cluster Incl AI850563 UI-M-BG1-aij-e-12-0-UI s1 Mus musculus cDNA, 3 end/clone = UI-M-BG1-aij-e-12-0-UI/ | P | 424.6 | P | 510.3 | P | 556.3 | P |
| | | clone_end = 3/gb = AI850563/gi = 5494469/ug = Mm.22727/len = 271/STRA = for | | | | | | | |
| 15 | 104524_at | Cluster Incl AI842825: UI-M-AP1-agi-h-07-0-UI s1 Mus musculus cDNA, 3 end/clone = UI-M-AP1-agi-h-07-0-UI/ | P | 2852.5 | P | 4574.3 | P | 4583.4 | P |
| | | clone_end = 3/gb = AI842825/gi = 5477038/ug = Mm 22986/len = 341/STRA = for | | | | | | | |
| 15 | 104559_at | Cluster Incl AI848162 UI-M-AH1-agq-c-02-0-UI s1 Mus musculus cDNA, 3 end/clone = UI-M-AH1-asg-c-02-0-UI/ | P | 926.7 | P | 2087.4 | P | 1774.8 | P |
| | | clone_end = 3/gb = AI848162/gi = 5492068/ug = Mm 23826/len = 492/STRA = for | | | | | | | |
| 15 | 104576_at | Cluster Incl AW212397: uo86e12.x1 Mus musculus cDNA, 3 end/clone = IMAGE-2649454/clone_end = 3/gb = AW212397/ | P | 1031.1 | P | 1512.1 | P | 1488.5 | P |
| | | gi = 6518452/ug = Mm 23941/len = 165/STRA = for | | | | | | | |
| 15 | 104710_at | Cluster Incl Y13231: Bcl2 homologous antagonist/killer/cds = (254,880)/gb = Y13231/gi = 2113878/ug = Mm 2443/len = 2005/ | P | 549.8 | P | 900.2 | P | 967 | P |
| | | STRA = for | | | | | | | |
| 15 | 94341_at | Cluster Incl D31967 Jumonji/cds = (210,3914)/gb = D31967/gi = 780143/ug = Mm.25059/len = 4071/STRA = for | P | 1171.1 | P | 1680.2 | P | 1312 | P |
| 15 | 94929_at | Cluster Incl M97590: Mouse tyrosine phosphatase (PTP-1), (EC2 7 1.112) mRNA/cds = UNKNOWN/gb = M97590/ | P | 641.9 | P | 1204.3 | P | 1112.3 | P |
| | | gi = 200581/ug = Mm 2668/len = 1664/STRA = for | | | | | | | |
| 15 | 94972_at | Cluster Incl AB026569 Mus musculus mRNA for MSSP, complete cds/cds = (261,1373)/gb = AB026569/gi = 4730905/ | P | 551.2 | P | 773.3 | P | 750.9 | P |
| | | ug = Mm 27230/len = 1650/STRA = for | | | | | | | |
| 15 | 95627_at | Cluster Incl AW046038 UI-M-BH1-akv-b-03-0-U1.s1 Mus musculus cDNA, 3 end/clone = UI-M-BH1-akv-b-03-0-UI/ | P | 307.9 | P | 884 | P | 856.3 | P |
| | | clone_end = 3/gb = AW046038/gi = 5906567/ug = Mm 28060/len = 433/STRA = for | | | | | | | |
| 15 | 96197_f_at | Cluster Incl AI851708 UI-M-BHO-aio-d-08-0-UI s1 Mus musculus cDNA, 3 end/clone = UI-M-BH0-aio-d-08-0-UI/ | P | 325.9 | P | 452 | P | 386.2 | P |
| | | clone_end = 3/gb = AI851708/gi = 5495609/ug = Mm.28553/len = 337/STRA = for | | | | | | | |
| 15 | 96220_at | Cluster Incl AW123157 UI-M-BH2 1-apd-c-10-0-UI.s1 Mus musculus cDNA, 3 end/clone = UI-M-BH2 1-apd-c-10-0-UI/ | P | 251 | P | 412.5 | P | 362 | P |
| | | clone_end = 3/gb = AW123157/gi = 6098687/ug = Mm 28709/len = 542/STRA = for | | | | | | | |
| 15 | 97375_at | Cluster Incl U70209 Mus musculus polycystic kidney disease 1 protein (Pkd1) mRNA, complete cds/cds = (315,13196)/ | P | 173.5 | P | 318.8 | P | 216 | P |
| | | gb = U70209/gi = 2138182/ug = Mm 2973/len = 14141/STRA = for | | | | | | | |
| 15 | 97426_at | Cluster Incl X98471: Epithelial membrane protein 1/cds = (0,482)/gb = X98471/gi = 1580765/ug = Mm.30024/len = 483/ | P | 2473.1 | P | 3251.1 | P | 3693.8 | P |
| | | STRA = for | | | | | | | |
| 15 | 97937_at | Cluster Incl AA611766: vo85108.r1 Mus musculus cDNA, 5 end/clone = IMAGE-1065927/clone_end = 5/gb = AA611766/ | P | 1099.7 | P | 1622.8 | P | 1241.3 | P |
| | | gi = 2461801/ug = Mm.30262/len = 161/STRA = rev | | | | | | | |
| 15 | 98432_at | Cluster Incl X99384: M musculus mRNA for paladin gene/cds = (46,2625)/gb = X99384/gi = 1545958/ug = Mm.3437/len = 4055/ | P | 142.1 | P | 245.7 | P | 233.3 | P |
| | | STRA = for | | | | | | | |
| 15 | 98436_s_at | Cluster Incl U54803 Caspase 3, apoptosis related cysteine protease/cds = (15,848)/gb = U54803/gi = 1518967/ | P | 160.4 | P | 310.5 | P | 240.4 | P |
| | | ug = Mm 34405/len = 1418/STRA = rev | | | | | | | |
| 15 | 98477_s_at | Cluster Incl L40632 Ankyrin 3, epithelial/cds = (56,5941)/gb = L40632/gi = 710548/ug = Mm 3526/len = 7084/STRA = for | P | 82.3 | P | 190.9 | P | 117.9 | P |
| 15 | 98984_I _at | Cluster Incl D50430 Mouse mRNA for glycerol-3-phosphate dehydrogenase, complete cds/cds = (96,2279)/gb = D50430/ | P | 248.2 | P | 387.6 | P | 358.8 | P |
| | | gi = 1339937/ug = Mm 3711/len = 2410/STRA = for | | | | | | | |
| 15 | 99054_at | Cluster Incl D16141: Lethal giant larvae homolog/cds = (21,3125)/gb = D16141/gi = 414350/ug = Mm 3940/len = 4279/ | P | 902.9 | P | 1108 | P | 1021.6 | P |
| | | STRA = rev | | | | | | | |
| 15 | 99458_I_at | Cluster Incl X70764 ELKL motif kinase/cds = (93,2417)/gb = X70764/gi = 57919/ug = Mm 4082/len = 2763/STRA = for | P | 2156.5 | P | 3723.3 | P | 3064.7 | P |
| 15 | 99934_at | Cluster Incl M80206 Poliovirus sensitivity/cds = (0,1403)/gb = M80206/gi = 199785/ug = Mm 4341/len = 1404/STRA = for | P | 436 | P | 602.4 | P | 693.2 | P |
| 15 | 100011_at | Cluster Incl AI851658: UI-M-BH0-ain-f-03-0-UI s1 Mus musculus cDNA, 3 end/clone = UI-M-BH0-ain-f-03-0-UI/ | P | 858.6 | P | 2235.6 | P | 1731.9 | P |
| | | clone_end = 3/gb = AI851658/gi = 5495564/ug = Mm 4549/len = 448/STRA = for | | | | | | | |
| 15 | 100027_s_at | Cluster Incl AI881987 uI56h05.x1 Mus musculus cDNA, 3 end/clone = IMAGE-2123097/clone_end = 3/gb = AI881987/ | P | 629.7 | P | 744.7 | P | 713.9 | P |
| | | gi = 5567139/ug = Mm 4609/len = 512/STRA = for | | | | | | | |
| 15 | 100029_at | Cluster Incl AW208667: uo62e02 x1 Mus musculus cDNA, 3 end/clone = IMAGE-2647130/clone_end = 3/gb = AW208667/ | P | 1161.8 | P | 1945.6 | P | 2159.4 | P |
| | | gi = 6514607/ug = Mm 4609/len = 474/STRA = for | | | | | | | |
| 15 | 100032_at | Cluster Incl X60136 Trans-acting transcription factor 1/cds = (0,307)/gb = X60136/gi = 54158/ug = Mm 4618/len = 454/ | P | 324.9 | P | 1547.7 | P | 885.3 | P |
| | | STRA = for | | | | | | | |
| 15 | 100401_at | Cluster Incl AA673981: vo87c03 r1 Mus musculus cDNA, 5 end/clone = IMAGE-1066084/clone_end = 5/gb = AA673981/ | P | 435.4 | P | 763.4 | P | 733.4 | P |
| | | gi = 2647263/ug = Mm 46401/len = 358/STRA = for | | | | | | | |
| 15 | 100420_at | Cluster Incl J03458 Filaggnn/cds = (0,1010)/gb = J03458/gi = 193307/ug = Mm 4685/len = 1488/STRA = for | P | 3434.1 | P | 6222.6 | P | 5575.4 | P |
| 15 | 100514_at | Cluster Incl M63660 Mouse G-alpha-13 protein mRNA, complete cds/cds = (128,1261)/gb = M63660/gi = 193378/ | P | 609.9 | P | 1058.6 | P | 981 | P |
| | | ug = Mm 5238/len = 2422/STRA = rev | | | | | | | |
| 15 | 101406_at | Cluster Incl AF026072 Mus musculus hydroxysteroid sulfotransferase (SULT2B) mRNA, complete cds/cds = (69,1085)/ | P | 629.6 | P | 740.9 | P | 806.2 | P |
| | | gb = AF026072/gi = 2570897/ug = Mm 7316/len = 1127/STRA = for | | | | | | | |
| 15 | 101448_at | Cluster Incl D50050: HGF-regulated tyrosine kinase substrate/cds = (42,2369)/gb = D50050/gi = 1089780/ug = Mm.7919/ | P | 1585.2 | P | 2039.8 | P | 1966.9 | P |
| | | len = 2458/STRA = for | | | | | | | |
| 15 | 101922_at | Cluster Incl AW123408 UI-M-BH2 1-apj-b-01-0-UI.s1 Mus musculus cDNA, 3 end/clone = UI-M-BH2.1-apj-b-01-0-UI/ | P | 529.1 | P | 749.6 | P | 798.5 | P |
| | | clone_end = 3/gb = AW123408/gi = 6098938/ug = Mm 92288/len = 410/STRA = for | | | | | | | |
| 15 | 102222_at | Cluster Incl AJ002730 Ubiquitously transcribed tetratricopeptide repeat gene, X Chromosome/cds = (0,4003)/gb = AJ002730/ | P | 422.6 | P | 1162.8 | P | 1032.7 | P |
| | | gi = 3021456/ug = Mm 10218/len = 4215/STRA = rev | | | | | | | |
| 15 | 102273_at | Cluster Incl M31226 Mouse immune suppressor factor TJ6 mRNA, complete cds/cds = (18,2585)/gb = M31226/gi = 198460/ | P | 225.2 | P | 350.9 | P | 329.4 | P |
| | | ug = Mm 1158/len = 2936/STRA = for | | | | | | | |
| 15 | 102691_at | Cluster Incl AI848518 UI-M-AP1-agf-e-02-0-UI s2 Mus musculus cDNA, 3 end/clone = UI-M-AP1-agf-e-02-0-UI/ | P | 773.6 | P | 1020.5 | P | 652.4 | P |
| | | clone _end _3/gb = AI848518/gi = 5492424/ug = Mm.14099/len = 361/STRA = for | | | | | | | |
| 15 | 102727_at | Cluster Incl X55573 Brain derived neurotrophic factor/cds = (77,826)/gb = X55573/gi = 287898/ug = Mm 1442/len = 927/ | P | 166.9 | P | 333.1 | P | 249.6 | P |
| | | STRA = for | | | | | | | |
| 15 | 103269_f_at | Cluster Incl AJ005350 Mus musculus of ZT2 gene encoding zinc finger protein 125/cds = (0,541)/gb = AJ005350/ | P | 150 | P | 411.1 | P | 308.6 | P |
| | | gi = 3646358/ug = Mm.20857/len = 2440/STRA = for | | | | | | | |
| 15 | 103288_at | Cluster Incl AF053062 Mus musculus receptor interacting protein 140 mRNA, complete cds/cds = (371,3856)/ | P | 181.6 | P | 600.6 | P | 571.2 | P |
| | | gb = AF053062/gi = 3820493/ug = Mm 20895/len = 4307/STRA = for | | | | | | | |
| 15 | 103484_at | Cluster Incl AA619442 vo84c10.r1 Mus musculus cDNA, 5 end/clone = IMAGE-1065810/clone_end = 5/gb = AA619442/ | P | 9.7 | P | 109.7 | P | 35.5 | P |
| | | gi = 2523318/ug = Mm.22133/len = 464/STRA = for | | | | | | | |
| 15 | 103709_at | Cluster Incl AA763466 vw54f05 r1 Mus musculus cDNA, 5 end/clone = IMAGE-1247649/clone_end = 5/gb = AA763466/ | P | 2204.1 | P | 3941.4 | P | 3938 | P |
| | | gi = 2813213/ug = Mm 24093/len = 379/STRA = for | | | | | | | |
| 15 | 103771_at | Cluster Incl AI837993 UI-M-AP0-abl-d-01-0-UI s1 Mus musculus cDNA, 3 end/clone = UI-M-AP0-abl-d-01-0-UI/ | P | 974.5 | P | 1782.6 | P | 1559 | P |
| | | clone_end = 3/gb = AI837993/gi = 54722067/ug = Mm 24777/len = 336/STRA = rev | | | | | | | |
| 15 | 103774_at | Cluster Incl AW108350: um29f06 x1 Mus musculus cDNA, 3 end/clone = IMAGE-2235971/clone_end = 3/gb = AW108350/ | P | 275.7 | P | 610.9 | P | 513.5 | P |
| | | gi = 6079150/ug = Mm 24790/len = 579/STRA = for | | | | | | | |
| 15 | 103978_at | Cluster Incl AA666669: vm50f06.r1 Mus musculus cDNA, 5 end/clone = IMAGE-1001699/clone_end = 5/gb = AA666669/ | P | 426.4 | P | 932.6 | P | 637.7 | P |
| | | gi = 2625370/ug = Mm 25787/len = 412/STRA = for | | | | | | | |
| 15 | 103994_at | Cluster Incl AI152867: ud93h11.r1 Mus musculus cDNA, 5 end/clone = IMAGE-1478469/clone_end = 5/gb = AI152867/ | P | 686.3 | P | 1482.7 | P | 1328.4 | P |
| | | gi = 3681336/ug = Mm 26266/len = 471/STRA = for | | | | | | | |
| 15 | 104158_at | Cluster Incl AW046671: UI-M-BH1-alm-a-09-0-UI s1 Mus musculus cDNA, 3 end/clone = UI-M-BH1-alm-a-09-0-UI/ | P | 105.1 | P | 298.2 | P | 214.2 | P |
| | | clone_end = 3/gb = AW046671/gi = 5907200/ug = Mm.27094/len = 240/STRA = for | | | | | | | |
| 15 | 104225_at | Cluster Incl AI645050 vs46d05 y1 Mus musculus cDNA, 5 end/clone = IMAGE-1149321/clone_end = 5/gb = AI645050/ | P | 295.5 | P | 793.6 | P | 571.1 | P |
| | | gi = 4723525/ug = Mm 27988/len = 373/STRA = rev | | | | | | | |
| 15 | 104407_at | Cluster Incl L25274 Mus musculus transmembrane glycoprotein (DM-GRASP) mRNA, 3 end/cds = (0,1075)/gb = L25274/ | P | 254.4 | P | 602.8 | P | 418.9 | P |
| | | gi = 452102/ug = Mm 2877/len = 1288/STRA = for | | | | | | | |
| 15 | 104433_at | Cluster Incl AI849682 UI-M-AL1-ahl-f-01-0-UI s1 Mus musculus cDNA, 3 end/clone = UI-M-AL1-ahl-f-01-0-UI/clone_end = 3/ | P | 303.9 | P | 487.2 | P | 520.6 | P |
| | | gb = AI849682/gi = 5493588/ug = Mm 29228/len = 245/STRA = rev | | | | | | | |
| 15 | 104461_at | Cluster Incl AW121773 UI-M-BH2 3-aoe-f-11-0-UI s1 Mus musculus cDNA, 3 end/clone = UI-M-BH2.3-aoe-f-11-0-UI/ | P | 233.2 | P | 300.1 | P | 266 | P |
| | | clone_end = 3/gb = AW121773/gi = 6097121/ug = Mm 29589/len = 166/STRA = for | | | | | | | |
| 15 | 104663_at | Cluster Incl D86177 Mouse mRNA for phosphatidylinositol 4-phosphate 5-kinase type l-beta, complete cds/cds = (269,1909)/ | P | 291.1 | P | 583.6 | P | 716.5 | P |
| | | gb = D86177/gi = 1622709/ug = Mm 3191/len = 2403/STRA = for | | | | | | | |
| 15 | 104682_at | Cluster Incl AI848983: UI-M-AJ1-ahd-d-05-0-UI s1 Mus musculus cDNA, 3 end/clone = UI-M-AJ1-ahd-d-05-0-UI/ | P | 604.7 | P | 777.4 | P | 1031.1 | P |
| | | clone_end = 3/gb = AI848983/gi = 5492889/ug = Mm.32884/len = 499/STRA = for | | | | | | | |
| 15 | 92195_at | Cluster Incl AB012273 Mus musculus mRNA for GPE1-BP (C/EBP gamma), complete cds/cds = (224,676)/gb = AB012273/ | P | 246.4 | P | 394.7 | P | 421.4 | P |
| | | gi = 2988367/ug = Mm 3402/len = 1185/STRA = for | | | | | | | |
| 15 | 92444_f_at | Cluster Incl X16493: Zinc finger protein 1/cds = (320,1594)/gb = X16493/gi = 55482/ug = Mm.4184/len = 1939/STRA = rev | P | 241.4 | P | 302.4 | P | 255.1 | P |
| 15 | 92452_at | Cluster Incl U03279: Phosphatidylinositol 3-kinase, catalytic, alpha polypeptide/cds = (0,3206)/gb = U03279/gi = 414994/ | P | 185.9 | P | 263.2 | P | 237.2 | P |
| | | ug = Mm 41943/len = 3207/STRA = rev | | | | | | | |
| 15 | 92703_at | Cluster Incl AI325791 mn19e09.y1 Mus musculus cDNA, 5 end/clone = IMAGE-538408/clone_end = 5/gb = AI325791/ | P | 118.3 | P | 615.4 | P | 518 | P |
| | | gi = 4060220/ug = Mm 45992/len = 434/STRA = for | | | | | | | |
| 15 | 92891_f_at | Cluster Incl X55318 Homeo box C9/cds = (348,1130)/gb = X55318/gi = 57878/ug = Mm 4765/len = 1434/STRA = for | P | 81.4 | P | 141.4 | P | 127.2 | P |
| 15 | 92903_at | Cluster Incl X78197 Transcription factor AP-2 beta/cds = (213,1559)/gb = X78197/gi = 496638/ug = Mm 4795/len = 1731/ | P | 298.8 | P | 589.8 | P | 580.2 | P |
| | | STRA = for | | | | | | | |
| 15 | 92918_at | Cluster Incl U66079 Mus musculus coagulation factor VII gene, complete cds/cds = (10,1350)/gb = U66079/gi = 1513221/ | P | 136.5 | P | 223.2 | P | 186 | P |
| | | ug = Mm.4827/len = 1843/STRA = for | | | | | | | |
| 15 | 93378_at | Cluster Incl X07439 Mouse mRNA for Hox-3 1 homeo gene/cds = (241,969)/gb = X07439/gi = 51406/ug = Mm 6167/ | P | 440.1 | P | 786.6 | P | 742.5 | P |
| | | len = 1470/STRA = for | | | | | | | |
| 15 | 93478_at | Cluster Incl AA612483: vo72g09.r1 Mus musculus cDNA, 5 end/clone = IMAGE-1064704/clone_end = 5/gb = AA612483/ | P | 244.6 | P | 295.9 | P | 284.4 | P |
| | | gi = 2461161/ug = Mm.7109/len = 384/STRA = rev | | | | | | | |
| 15 | 93938_at | Cluster Incl U17132: Zinc transporter 1/cds = (147,1658)/gb = U17132/gi = 577840/ug = Mm.9024/len = 1720/STRA = for | P | 309.8 | P | 625.4 | P | 496 | P |
| 15 | 94127_at | Cluster Incl Y15910 Mus musculus mRNA for dia protein/cds = (0,1049)/gb = Y15910/gi = 3171941/ug = Mm.10763/len = 1330/ | P | 98.5 | P | 179.2 | P | 251.4 | P |
| | | STRA = for | | | | | | | |
| 15 | 94689_at | Cluster Incl C79248 C79248 Mus musculus cDNA, 3 end/clone = J0063C04/clone_end = 3/gb = C79248/gi = 2519578/ | P | 484.1 | P | 1156.4 | P | 834 | P |
| | | ug = Mm 13291/len = 476/STRA = rev | | | | | | | |
| 15 | 94767_at | Cluster Incl AB028894 Mus musculus Rps11, U35 genes for ribosomal protein S11 and U35 snoRNA, complete cds and | P | 2960.4 | P | 5275.6 | P | 4868.6 | P |
| | | sequence/cds = (22,498)/gb = AB028894/gi = 6552367/ug = Mm 164/len = 551/STRA = for | | | | | | | |
| 15 | 95305_at | Cluster Incl AA756275 vv40d02 r1 Mus musculus cDNA, 5 end/clone = IMAGE-1224867/clone_end = 5/gb = AA756275/ | P | 249 | P | 608.3 | P | 336.4 | P |
| | | gi = 2803473/ug = Mm 20012/len = 526/STRA = for | | | | | | | |
| 15 | 95359_at | Cluster Incl M18186 Mouse 84 kD heat shock protein mRNA, complete cds/cds = (94,2268)/gb = M18186/gi = 194024/ | P | 1759.4 | P | 3192.1 | P | 3140 | P |
| | | ug = Mm 2180/len = 2509/STRA = for | | | | | | | |
| 15 | 95385_at | Cluster Incl AI643119 mm55a03 y1 Mus musculus cDNA, 5 end/clone = IMAGE-532300/clone_end = 5/gb = AI643119/ | P | 284.2 | P | 477.9 | P | 503.2 | P |
| | | gi = 4721594/ug = Mm 23210/len = 285/STRA = rev | | | | | | | |
| 15 | 95393_at | Cluster Incl AI503362: vn86g08.x1 Mus musculus cDNA, 3 end/clone = IMAGE-1038878/clone_end = 3/gb = AI503362/ | P | 248.9 | P | 375.9 | P | 357.3 | P |
| | | gi = 4401213/ug = Mm 23817/len = 425/STRA = for | | | | | | | |
| 15 | 95944_at | Cluster Incl AV299153 AV299153 Mus musculus cDNA, 3 end/clone = 5730466C10/clone_end = 3/gb = AV299153/ | P | 258.8 | P | 708.6 | P | 473.1 | P |
| | | gi = 6331610/ug = Mm 24891/len = 273/STRA = rev | | | | | | | |
| 15 | 96522_at | Cluster Incl M19381 Calmodulin/cds = (186,635)/gb = M19381/gi = 469421/ug = Mm.2648/len = 798/STRA = for | P | 3236.1 | P | 4920.4 | P | 4343.9 | P |
| 15 | 96532_at | Cluster Incl AA212897: mw62a09 r1 Mus musculus cDNA, 5 end/clone = lMAGE-677176/clone_end = 5/gb = AA212897/ | P | 84 | P | 194.7 | P | 225.6 | P |
| | | gi = 1811645/ug = Mm 27297/len = 429/STRA = for | | | | | | | |
| 15 | 96542_at | Cluster Incl M62606 Surfeit gene 4/cds = (105,914)/gb = M62606/gi = 201091/ug = Mm 2795/len = 2781/STRA = for | P | 1420.9 | P | 2345.4 | P | 2260.5 | P |
| 15 | 96573_at | Cluster Incl M21495 Mouse cytoskeletal gamma-actin mRNA, complete cds/cds = (39,1186)/gb = M21495/gi = 191656/ | P | 4550.5 | P | 8384.1 | P | 7871.8 | P |
| | | ug = Mm 29913/1en = 1868/STRA = for | | | | | | | |
| 15 | 96575_at | Cluster Incl U67771: Mus musculus ribosomal protein L8 (RPL8) mRNA, complete cds/cds = (21,794)/gb = U67771/ | P | 2472.4 | P | 5096.8 | P | 4263.3 | P |
| | | gi = 1527177/ug = Mm 30066/len = 842/STRA = for | | | | | | | |
| 15 | 97695_s_at | Cluster Incl M29015.Mouse ribosomal protein L7 (rpL7) gene, complete cds/cds = (0,812)/gb = M29015/gi = 200784/ | P | 4395.3 | P | 8060.5 | P | 8335.7 | P |
| | | ug = Mm 37835/len = 813/STRA = for | | | | | | | |
| 15 | 97696_r_at | Cluster Incl M29015 Mouse ribosomal protein L7 (rpL7) gene, complete cds/cds = (0,812)/gb = M29015/gi = 200784/ | P | 2265.1 | P | 4355.3 | P | 4094.3 | P |
| | | ug = Mm 37835/len = 813/STRA = for | | | | | | | |
| 15 | 97750_at | Cluster Incl X06406 Mouse mRNA for tranalational controlled 40 kDa polyyeptide p40/cds = (77,964)/gb = X06406/gi = 52852/ | P | 1844.5 | P | 2768.8 | P | 2532.7 | P |
| | | ug = Mm 4071/len = 1031/STRA = for | | | | | | | |
| 15 | 97751_f_at | Cluster Incl AI835771 UI-M-AI0-aaq-a-05-0-UI s1 Mus musculus cDNA, 3 end/clone = UI-M-AI0-aaq-a-05-0-UI/ | P | 1505.1 | P | 2506.8 | P | 2305.9 | P |
| | | clone_end = 3/gb = AI835771/gi = 5469984/ug = Mm 40749/len = 324/STRA = rev | | | | | | | |
| 15 | 98313_at | Cluster InCL AB025413 Mus musculus mRNA for Ten-m4, complete cds/cds = (187,8502)/gb = AB025413/gi = 4760781/ | P | 133.5 | P | 296.2 | P | 162.6 | P |
| | | ug = Mm 42204/len = 8585/STRA = for | | | | | | | |
| 15 | 98319_at | Cluster Incl AJ000328 Mus musculus mRNA tor type 2 desmoglein/cds = (0,245)/gb = AJ000328/gi = 2764500/ | P | 131.5 | P | 199.9 | P | 188.3 | P |
| | | ug = Mm 42229/len = 745/STRA = for | | | | | | | |
| 15 | 98331_at | Cluster Incl X52046: Procollagen, type III, alpha 1/cds = (117,4511)/gb = X52046/gi = 575321/ug = Mm 4271/len = 4766/ | P | 3630.7 | P | 5863.9 | P | 6027.4 | P |
| | | STRA = for | | | | | | | |
| 15 | 98333_at | Cluster Incl AF100956 Mus musculus major histocompatibility locus class II region, Fas-binding protein Daxx (DAXX) gene, | P | 2525.9 | P | 4270.8 | P | 4027 | P |
| | | partial cds, Bing1 (BING1), tapasin (tapasin), RalGDS-like factor (RLF), KE2 (KE2), BING4 (BING4), beta1, 3-galactosyl | | | | | | | |
| | | transferase (be/STRA = for | | | | | | | |
| 15 | 98342_at | Cluster Incl M93980 Mouse 24.6 kda protein mRNA, complete cds/cds = (41,685)/gb = M93980/gi = 410741/ug = Mm 43239/ | P | 3242.6 | P | 6331.7 | P | 5732.6 | P |
| | | len = 757/STRA = for | | | | | | | |
| 15 | 98346_at | Cluster Incl AI593759 v171b05 x1 Mus musculus cDNA, 3 end/clone = IMAGE-1176561/clone_end = 3/gb = AI593759/ | P | 123.6 | P | 287.4 | P | 253.3 | P |
| | | gi = 4602807/ug = Mm 43514/len = 296/STRA = for | | | | | | | |
| 15 | 98369_f_at | Cluster Incl AW048053: UI-M-BH1-alv-g-06-0-U1.s1 Mus musculus cDNA, 3 end/clone = UI-M-BH1-alv-g-06-0-UI/ | P | 2467.9 | P | 3836.8 | P | 3983.1 | P |
| | | clone_end = 3/gb = AW048053/gi = 5908582/ug = Mm 44493/len = 532/STRA = rev | | | | | | | |
| 15 | 98759_f_at | Cluster Incl M28727 Tubulin alpha 2/cds = (0,1058)/gb = M28727/gi = 202224/ug = Mm 4591/len = 1198/STRA = for | P | 2166.3 | P | 4486.9 | P | 4411 | P |
| 15 | 98767_at | Cluster Incl AI842603: UI-M-AQ1-aed-b-11-0-UI s1 Mus musculus cDNA, 3 end/clone = UI-M-AQ1-aed-b-11-0-UI/ | P | 258.3 | P | 411.6 | P | 332.1 | P |
| | | clone_end = 3/gb = AI842603/gi = 5476818/ug = Mm 463/len = 349/STRA = rev | | | | | | | |
| 15 | 98817_at | Cluster Incl Z29532 M musculus mRNA for follistatin/cds = (0,1031)/gb = Z29532/gi = 488368/ug = Mm 4913/len = 1123/ | P | 413.5 | P | 819.1 | P | 616.9 | P |
| | | STRA = for | | | | | | | |
| 15 | 98862_at | Cluster Incl U61969 Mus musculus Wn110a mRNA, complete cds/cds = (662,1915)/gb = U61969/gi = 1546012/ug = Mm 5130/ | P | 251 | P | 316.4 | P | 349.3 | P |
| | | len = 2487/STRA = rev | | | | | | | |
| 15 | 99327_at | Cluster Incl D30785 Mouse mRNA for neuropsin, complete cds/cds = (486,1268)/gb = D30785/gi = 1648847/ug = Mm 5193/ | P | 590.4 | P | 943.6 | P | 893.6 | P |
| | | len = 1322/STRA = for | | | | | | | |
| 15 | 99335_at | Cluster Incl J05277: Hexokinase 1/cds = (4,2760)/gb = J05277/gi = 193841/ug = Mm 5290/len = 4198/STRA = for | P | 2581.2 | P | 4679 | P | 3952.5 | P |
| 15 | 99336_at | Cluster Incl U78085: Mus musculus ribosomal protein S5 mRNA, complete cds/cds = (65,679)/gb = U78085/gi = 1685070/ | P | 3575.7 | P | 6304.2 | P | 5766.3 | P |
| | | ug = Mm.5291/len = 763/STRA = for | | | | | | | |
| 15 | 99340_at | Cluster Incl D29802 Guanine nucleotide binding protein, beta-2, related sequence 1/cds = (98,1051)/gb = D29802/ | P | 3344.6 | P | 6668.9 | P | 5323.4 | P |
| | | gi = 475011/ug = Mm 5305/len = 1092/STRA = for | | | | | | | |
| 15 | 100297_at | Cluster Incl AA693125 vr57c08 s1 Mus musculus cDNA, 5 end/clone = IMAGE-1124750/clone_end = 5/gb = AA693125/ | P | 563.2 | P | 1256.5 | P | 1331.2 | P |
| | | gi = 2694063/ug = Mm 86411/len = 265/STRA = for | | | | | | | |
| 15 | 100307_at | Cluster Incl AA002843 mg38c08 r1 Mus musculus cDNA, 5 end/cone = IMAGE-426062/clone_end = 5/gb = AA002343/ | P | 177.5 | P | 260.8 | P | 219.5 | P |
| | | gi = 1446281/ug = Mm 86787/len = 235/STRA = for | | | | | | | |
| 15 | 100343_f_at | Cluster Incl M28729 Tubulin alpha1/cds = (0,596)/gb = M28729/gi = 202222/ug = Mm 88110/len = 786/STRA = for | P | 2554.6 | P | 4613.1 | P | 4131.2 | P |
| 15 | 100379_f_at | Cluster Incl AI837905 UI-M-AL0-abq-c-01-0-UI s1 Mus musculus cDNA, 3 end/clone = UI-M-AL0-abq-c-01-0-UI/ | P | 2962.7 | P | 4999.7 | P | 4732.3 | P |
| | | clone_end = 3/gb = AI837905/gi = 5472118/ug = Mm.89082/len = 223/STRA = rev | | | | | | | |
| 15 | 100380_at | Cluster Incl X91866 M musculus mRNA for H3 3A variant histone/cds = UNKNOWN/gb = X91866/gi = 1022324/ | P | 3187.5 | P | 4575.2 | P | 4517.6 | P |
| | | ug = Mm 89136/len = 1304/STRA = for | | | | | | | |
| 15 | 100758_at | Cluster Incl AF110520 Mus musculus major histocompatibility complex region NG27, NG28, RPS28, NADH | P | 2741.8 | P | 5107.6 | P | 4743.3 | P |
| | | oxidoreductase, NG29, KIFC1, Fas-binding protein, BING1, tapasin, RalGDS-like, KE2, BING4, beta 1,3-galactosyl | | | | | | | |
| | | transferase, and RPS18 genes, complete cds, S/STRA = for | | | | | | | |
| 15 | 100780_at | Cluster Incl M73436 Ribosomal protein S4, X-linked/cds = (6,797)/gb = M73436/gi = 200863/ug = Mm.66/len = 885/STRA = for | P | 3973.8 | P | 7189.3 | P | 6629.6 | P |
| 15 | 101255_at | Cluster Incl X51703 Mouse mRNA for ubiquitin/cds = (104,1021)/gb = X51703/gi = 55117/ug = Mm.235/len = 1172/STRA = for | P | 2318.6 | P | 5582.6 | P | 4806.4 | P |
| 15 | 101732_at | Cluster Incl M12039 Mouse DNA homologous to the Drosophila per locus/cds = (0,2018)/gb = M12039/gi = 200288/ | P | 459.2 | P | 586.1 | P | 580.1 | P |
| | | ug = Mm 57054/len = 2019/STRA = for | | | | | | | |
| 15 | 101781_f_at | Cluster Incl V00754 Mouse gene coding for embryonic H3 histone/cds = (0,407)/gb = V00754/gi = 51297/ug = Mm 57118/ | P | 1047.9 | P | 1487.8 | P | 1388.9 | P |
| | | len = 408/STRA = for | | | | | | | |
| 15 | 101787_f_at | Cluster Incl X16672: Mouse DNA for type IIB intracistemal A-particle element encoding integrase and gag/cds = (0,323)/ | P | 2652 | P | 4657.2 | P | 3914.7 | P |
| | | gb = X16672/gi = 51499/ug = Mm 57126/len = 324/STRA = for | | | | | | | |
| 15 | 102599_at | Cluster Incl X06407 Mouse mRNA for 21 kd polypeptide under translational control/cds = (100,618)/gb = X06407/gi = 52848/ | P | 2233.3 | P | 4712.7 | P | 3684.9 | P |
| | | ug = Mm 254/len = 816/STRA = for | | | | | | | |
| 15 | 94662_at | Cluster Incl AA409766 EST01475 Mus musculus cDNA, 3 end/clone = C0010D03/clone_end = 3/gb = AA409766/ | P | 212.3 | P | 514 | P | 567.4 | P |
| | | gi = 2066390/ug = Mm 24471/len = 316/STRA = rev | | | | | | | |
| 15 | 95184_f_at | Cluster Incl AA419684 vc51g02 r1 Mus musculus cDNA, 3 end/clone = IMAGE-778130/clone_end = 3/gb = AA419684/ | P | 300.2 | P | 556.5 | P | 487.6 | P |
| | | gi = 2080962/ug = Mm 24548/len = 243/STRA = rev | | | | | | | |
| 15 | 96962_at | Cluster Incl X81987 M musculus mRNA for TAX responsive element binding protein 107/cds = (27,890)/gb = X81987/ | P | 2787.4 | P | 4917.9 | P | 4639.1 | P |
| | | gi = 695637/ug = Mm 588/len = 950/STRA = for | | | | | | | |
| 15 | 97017_f_at | Cluster Incl AW214439 uo48c01 X1 Mus musculus cDNA, 3 end/clone = IMAGE-2645760/clone_end = 3/gb = AW214439/ | P | 1401.2 | P | 2046.5 | P | 2078.4 | P |
| | | gi = 6520962/ug = Mm 89021/len = 650/STRA = rev | | | | | | | |
| 15 | 97077_f_at | Cluster Incl AW213991 uo46d02 x1 Mus musculus cDNA, 3 end/clone = IMAGE-2645571/clone_end = 3/gb = AW213991/ | P | 1329.3 | P | 1976.2 | P | 1879.4 | P |
| | | gi = 6520344/ug = Mm B9381/len = 468/STRA = rev | | | | | | | |
| 15 | 97559_at | Cluster Inc M76131 Mouse elongation factor2 (ef-2) mRNA, 3 end/cds = (0,806)/gb = M76131/gi = 192988/ug = Mm 70787/ | P | 2822 | P | 4770.7 | P | 4766.3 | P |
| | | len = 1179/STRA = for | | | | | | | |
| 15 | 97594_r_at | Cluster Incl C79694.C79694 Mus musculus cDNA, 3 end/clone = J0070F03/clone_end = 3/gb = C79694/gi = 2520024/ | P | 1009.1 | P | 1322.2 | P | 1513.8 | P |
| | | ug = Mm 89577/len = 439/STRA = rev | | | | | | | |
| 15 | 97647_at | Cluster Incl M11408 Ribosomal protein S16/cds = (52,489)/gb = M11408/gi = 435544/ug = Mm 702/len = 547/STRA = for | P | 2969.2 | P | 5931.6 | P | 5124.1 | P |
| 15 | 98168_at | Cluster Incl M14689 Mouse surfeit locus surfeit 3 protein gene/cds = (0,800)/gb = M14689/gi = 201081/ug = Mm.725/len = 801/ | P | 2601.1 | P | 4574 | P | 3964.4 | P |
| | | STRA = for | | | | | | | |
| 15 | 98169_s_at | Cluster Incl AU020229 AU020229 Mus musculus cDNA, 3 end/clone = J0529B09/clone_end = 3/gb = AU020229/ | P | 190.8 | P | 415.9 | P | 302 | P |
| | | gi = 3375813/ug = Mm 26206/len = 442/STRA = rev | | | | | | | |
| 15 | 98254_f_at | Cluster Incl AW209004 un98106.x1 Mus musculus cDNA, 3 end/clone = IMAGE-2581955/clone_end = 3/gb = AW209004/ | P | 3011.7 | P | 5095.7 | P | 4666.5 | P |
| | | gi = 6514944/ug = Mm 91451/len = 693/STRA = rev | | | | | | | |
| 15 | 100685_at | Cluster Incl M10937: Keratin complex 2, basic, gene 1/cds = (67,1953)/gb = M10937/gi = 4159805/ug = Mm.14245/len = 2341/ | P | 1482.2 | P | 2903 | P | 2553.2 | P |
| | | STRA = for | | | | | | | |
| 15 | 100694_at | Cluster Incl U29402 Mus musculus acidic ribosomal phosphoprotein P1 mRNA, complete cds/cds = (110,454)/gb = U29402/ | P | 1641.4 | P | 3928.8 | P | 3161.6 | P |
| | | gi = 902557/ug = Mm 14843/len = 495/STRA = for | | | | | | | |
| 15 | 100710_at | Cluster Incl Z14044 Valosin containing protein/cds = (161,2581)/gb = Z14044/gi = 55216/ug = Mm 18921/len = 3143/ | P | 2308.5 | P | 3267.1 | P | 2345.8 | P |
| | | STRA = for | | | | | | | |
| 15 | 100711_at | Cluster Incl U12403 Mus musculus Csa-19 mRNA, complete cds/cds = (15,668)/gb = U12403/gi = 531168/ug = Mm 2424/ | P | 3381.8 | P | 6070.6 | P | 5309.9 | P |
| | | len = 700/STRA = for | | | | | | | |
| 15 | 100718_at | Cluster Incl X56135: Mouse mRNA for prothymosin alpha/cds = (170,505)/gb = X56135/gi = 54796/ug = Mm.19187/len = 1191/ | P | 4880.4 | P | 8669 | P | 7544.9 | P |
| | | STRA = for | | | | | | | |
| 15 | 100720_at | Cluster Incl X65553 M. musculus mRNA for poly(A) binding protein/cds = (253,2163)/gb = X65553/gi = 53753/ug = Mm 2642/ | P | 3209.5 | P | 5143.6 | P | 4515.2 | P |
| | | len = 2244/STRA = for | | | | | | | |
| 15 | 100727_at | Cluster Incl X74856 M. musculus L28 mRNA for ribosomal protein L28/cds = (70,483)/gb = X74856/gl = 488834/ug = Mm 3111/ | P | 2663.8 | P | 4903.6 | P | 4960.3 | P |
| | | len = 555/STRA = for | | | | | | | |
| 15 | 100729_at | Cluster Incl X80699: M. musculus L26 mRNA/cds = (47,484)/gb = X80699/gi = 565623/ug = Mm 3229/len = 545/STRA = for | P | 2746.9 | P | 4780.8 | P | 4301 | P |
| 15 | 100732_at | Cluster Incl X73829.M. musculus mRNA for ribosomal protein S8/cds = (6,632)/gb = X73829/gi = 313297/ug = Mm 3381/ | P | 3959.5 | P | 6903 | P | 6558.5 | P |
| | | len = 671/STRA = for | | | | | | | |
| 15 | 100734_at | Cluster Incl Y00225 Murine mRNA for J1 protein, yeast ribosomal protein L3 homologue/cds = (9,1220)/gb = Y00225/ | P | 3740.4 | P | 8600.3 | P | 6200.5 | P |
| | | gi = 52740/ug = Mm.3486/len = 1276/STRA = for | | | | | | | |
| 15 | 100753_at | Cluster Incl L01062 Mouse ATP synthase alpha subunit, complete cds/cds = (38,1699)/gb = L01062/gi = 192089/ | P | 3326.9 | P | 5055.2 | P | 4317.4 | P |
| | | ug = Mm 4069/len = 2182/STRA = for | | | | | | | |
| 15 | 101128_at | Cluster Incl L06234: Mus musculus dihydropyridine sensitive skeletal muscle calcium channel mRNA, complete cds/ | P | 163.9 | P | 384 | P | 369 | P |
| | | cds = (116,4171)/gb = L06234/gi = 192323/ug = Mm.4418/len = 6010/STRA = for | | | | | | | |
| 15 | 101129_at | Cluster Incl X83590: M. musculus mRNA for ribosomal protein L5, 3 end/cds = (0,278)/gb = X83590/gi = 619503/ug = Mm.4419/ | P | 2126.9 | P | 3199.1 | P | 3012.7 | P |
| | | len = 349/STRA = for | | | | | | | |
| 15 | 101130_at | Cluster Incl X58251 Procollagen, type I, alpha 2/cds = (79,4200)/gb = X58251/gi = 50488/ug = Mm 4482/len = 4270/STRA = for | P | 3122.7 | P | 5032.6 | P | 5106 | P |
| 15 | 101137_at | Cluster Incl X76772: M musculus mRNA for ribosomal protein S3/cds = (5,736)/gb = X76772/gi = 439521/ug = Mm 4696/ | P | 2490.9 | P | 4241.6 | P | 3778.8 | P |
| | | len = 818/STRA = for | | | | | | | |
| 15 | 101165_at | Cluster Incl W91762 MTA D08 085 A Mus musculus cDNA, 3 end/clone = MTA D08 085/clone_end = 3/gb = W91762/ | P | 971 | P | 1343.9 | P | 1307.6 | P |
| | | gi = 1407931/ug = Mm 5000/len = 231/STRA = rev | | | | | | | |
| 15 | 101179_at | Cluster Incl D50494.D-E-A-D (aspartate-glutamate-alanine-aspartate) box polypeptide 6/cds = (258,1709)/gb = D50494/ | P | 1782.7 | P | 3014.4 | P | 3210.7 | P |
| | | gi = 940407/ug = Mm 5082/len = 2710/STRA = for | | | | | | | |
| 15 | 101207_at | Cluster Incl X52803 Mouse mRNA for cyclophilin (EC 5.2.1.8)/cds = (41,535)/gb = X52803/gi = 50620/ug = Mm 5246/len = 736/ | P | 3563.4 | P | 7835.9 | P | 6975.8 | P |
| | | STRA = for | | | | | | | |
| 15 | 101212_at | Cluster Incl AF043285 Mus musculus ribosomal protein S7 (rpS7) gene, complete cds/cds = (79,663)/gb = AF043285/ | P | 2647.7 | P | 5210.4 | P | 4250.6 | P |
| | | gi = 2811283/ug = Mm 5281/len = 682/STRA = for | | | | | | | |
| 15 | 101214_f_at | Cluster Incl M32599 Glyceraldehyde-3-phosphate dehydrogenase/cds = (46,1047)/gb = M32599/gi = 193423/ug = Mm 5289/ | P | 2562.5 | P | 4097.8 | P | 4367.3 | P |
| | | len = 1228/STRA = for | | | | | | | |
| 15 | 101664_at | Cluster Incl Z83368 M musculus RPS3a gene/cds = (25,819)/gb = Z83368/gi = 1841931/ug = Mm 6957/len = 864/STRA = for | P | 3733.6 | P | 8000 | P | 6697.6 | P |
| 15 | 101680_at | Cluster Incl X05021 Munne mRNA with homology to yeast L29 ribosomal protein gene/cds = (22,468)/gb = X05021/ | P | 3878 | P | 5832.9 | P | 6078 | P |
| | | gi = 50320/ug = Mm 7277/len = 507/STRA = for | | | | | | | |
| 15 | 102098_at | Cluster Incl AI415065 mb90h11.x1 Mus musculus cDNA, 3 end/clone = IMAGE-336741/clone_end = 3/gb = AI415065/ | P | 2818.8 | P | 4779.7 | P | 4312.8 | P |
| | | gi = 4258569/ug = Mm 42402/len = 278/STRA = rev | | | | | | | |
| 15 | 102109_at | Cluster Incl U28917 Mus musculus 60S ribosomal protein (A52) mRNA, complete cds/cds = (20,661)/gb = U28917/ | P | 2413.2 | P | 4820.8 | P | 4501.5 | P |
| | | gi = 899444/ug = Mm 42578/len = 704/STRA = for | | | | | | | |
| 15 | 102126_at | Cluster Incl X15962 Mouse mRNA for ribosomal protein S12/cds = (56,454)/gb = X15962/gi = 54005/ug = Mm 43452/len = 470/ | P | 2825.4 | P | 4294.8 | P | 3793.8 | P |
| | | STRA = for | | | | | | | |
| 15 | 92545_f_at | Cluster Incl AB006361.Mus musculus mRNA for prostaglandin D synthetase, complete cds/cds = (0,629)/gb = AB006361/ | P | 137.5 | A | 505.4 | P | 405 | P |
| | | gi = 2317285/ug = Mm.1008/len = 789/STRA = for | | | | | | | |
| 15 | 94241_at | Cluster Incl AI837229 UI-M-AK0-ade-e-06-0-UI.s1 Mus musculus cDNA, 3 end/clone = UI-M-AK0-ade-e-06-0-UI/ | P | 218.8 | P | 269 | P | 178.4 | A |
| | | clone_end = 3/gb = AI837229/gi = 5471482/ug = Mm.2235/len = 492/STRA = for | | | | | | | |
| 15 | 97318_at | Cluster Incl AI846197 UI-M-AM1-agb-e-02-0-UI s1 Mus musculus cDNA, 3 end/clone = UI-M-AM1-agb-e-02-0-UI/ | P | 119.1 | P | 150.2 | P | 161 | P |
| | | clone_end = 3/gb = AI846197/gi = 5490103/ug = Mm 28109/len = 310/STRA = for | | | | | | | |
| 15 | 96286_at | Cluster Incl AW060996 UI-M-BH1-amn-d-01-0-UI.s1 Mus musculus cDNA, 3 end/clone = UI-M-BH1-amn-d-01-0-UI/ | P | 592.6 | P | 650.7 | P | 665 | A |
| | | clone_end = 3/gb = AW060996/gi = 6008747/ug = Mm 29877/len = 542/STRA = for | | | | | | | |
| 15 | 97483_at | Cluster Incl M62952: Mus musculus ribosomal protein L19, complete cds/cds = (0,590)/gb = M62952/gi = 198642/ | P | 2083 | P | 4059.7 | P | 3362.9 | P |
| | | ug = Mm 30806/len = 673/STRA = for | | | | | | | |
| 15 | 99654_s_at | Cluster Incl AA726579 vu93a07 r1 Mus musculus cDNA, 5 end/clone = IMAGE-1209684/clone_end = 5/gb = AA726579/ | P | 1853.7 | A | 4655.1 | P | 3424.5 | P |
| | | gi = 2744286/ug = Mm 43751/len = 427/STRA = for | | | | | | | |
| 15 | 102425_at | Cluster Incl U61362 Mus musculus groucho-related gene 1 protein (Grg1) mRNA, complete cds/cds = (54,2366)/ | P | 109.9 | P | 167.4 | P | 192.5 | P |
| | | gb = U61362/gi = 1420928/ug = Mm 12823/len = 2381/STRA = for | | | | | | | |
| 15 | 94429_at | Cluster Incl L26479 Eukaryotic translation elongation factor 1 alpha 2/cds = (133,1524)/gb = L26479/gi = 1220409/ | P | 615.5 | P | 1113.1 | P | 1092.4 | A |
| | | ug = Mm 2645/len = 1782/STRA = for | | | | | | | |
| 15 | 98028_at | Cluster Incl M63849 Twist gene homolog, (Drosophila)/cds = (166,786)/gb = M63649/gi = 202243/ug = Mm 3280/len = 1485/ | A | 186.1 | P | 270.1 | P | 241.7 | P |
| | | STRA = for | | | | | | | |
| 15 | 101363_at | Cluster Incl AI644801 vb18e06 y1 Mus musculus cDNA, 5 end/clone = IMAGE-749314/clone_end = 5/gb = AI644801/ | P | 618.1 | P | 1058.7 | P | 854.2 | A |
| | | gi = 4723276/ug = Mm 68974/len = 281/STRA = for | | | | | | | |
| 15 | 103680_at | Cluster Incl AA683850: vr05102 r1 Mus musculus cDNA, 5 end/clone = IMAGE-1110939/clone_end = 5/gb = AA683850/ | P | 25.5 | P | 250.1 | P | 274.6 | P |
| | | gi = 2670436/ug = Mm 23884/len = 535/STRA = for | | | | | | | |
| 15 | 103926_at | Cluster Incl AV380793: AV380793 Mus musculus cDNA, 3 end/clone = 9230102C24/clone_end = 3/gb = AV380793/ | P | 94.6 | P | 345.5 | P | 318 | P |
| | | gi = 6429870/ug = Mm.25082/len = 255/STRA = rev | | | | | | | |
| 15 | 92388_at | Cluster Incl AW045528 UI-M-BH1-aks-c-03-0-UI s1 Mus musculus cDNA, 3 end/clone = UI-M-BH1-aks-c-03-0-UI/ | P | 645.2 | P | 845.2 | P | 568.4 | P |
| | | clone_end = 3/gb = AW045528/gi = 5906057/ug = Mm 4055/len = 389/STRA = for | | | | | | | |
| 15 | 92527_at | Cluster Incl Z50190 Adenylate cyclase 9/cds = (42,4103)/gb = Z50190/gi = 1729772/ug = Mm 4294/len = 4457/STRA = for | P | 227 | P | 303.8 | P | 349.3 | P |
| 15 | 92927_at | Cluster Incl L10426 Mus musculus ets-related protein 81 (ER81) mRNA, complete cds/cds = (200,1633)/gb = L10426/ | P | 75.5 | P | 123.7 | P | 127.8 | P |
| | | gi = 193195/ug = Mm 4866/len = 2499/STRA = rev | | | | | | | |
| 15 | 93459_s_at | Cluster Incl AW122897: UI-M-BH2.1-apa-d-11-0-UI s1 Mus musculus cDNA, 3 end/clone = UI-M-BH2 1-apa-d-11-0-UI/ | P | 491.3 | P | 1193 | P | 1087.9 | P |
| | | clone_end = 3/gb = AW122897/gi = 6098427/ug = Mm 68712/len = 409/STRA = for | | | | | | | |
| 15 | 93964_s_at | Cluster Incl AF038995 Mus musculus putative RNA helicase RCK mRNA, partial cds/cds = (0,1359)/gb = AF038995/ | P | 288.9 | P | 612.5 | P | 533.7 | P |
| | | gi = 2745893/ug = Mm 97095/len = 1360/STRA = for | | | | | | | |
| 15 | 94766_at | Cluster Incl M17878 Mouse eEF-Tu gene encoding elongation factor Tu, 5 end/cds = (80,404)/gb = M17878/gi = 192991/ | P | 2086.3 | A | 3969.5 | P | 3686.6 | P |
| | | ug = Mm.16317/len = 405/STRA = for | | | | | | | |
| 15 | 95883_at | Cluster Incl AW048355: UI-M-BH1-akk-f-02-0-UI s1 Mus musculus cDNA, 3 end/clone = UI-M-BH1-akk-f-02-0-UI/ | P | 143.6 | P | 246.9 | P | 213.2 | P |
| | | clone_end = 3/gb = AW048355/gi = 5908884/ug = Mm 23966/len = 483/STRA = rev | | | | | | | |
| 15 | 95964_at | Cluster Incl C77404 C77404 Mus musculus cDNA, 3 end/clone = J0030E06/clone_end = 3/gb = C77404/gi = 2517734/ | P | 88.6 | A | 288.2 | P | 204.7 | P |
| | | ug = Mm 24954/len = 508/STRA = rev | | | | | | | |
| 15 | 96493_at | Cluster Incl AA682091 vu13g02 s1 Mus musculus cDNA, 3 end/clone = IMAGE-1180562/clone_end = 3/gb = AA682091/ | P | 584.7 | P | 771.6 | P | 839.5 | A |
| | | gi = 2663123/ug = Mm 25192/len = 423/STRA = rev | | | | | | | |
| 15 | 99888_at | Cluster Incl U83190 Zonadhesin/cds = (0,1541)/gb = U83190/gi = 1932795/ug = Mm 7984/len = 1619/STRA = for | A | 374.8 | P | 442.3 | P | 396.6 | P |
| 15 | 100775_at | Cluster Incl C81070: C81070 Mus musculus cDNA, 3 end/clone = J0093A10/clone_end = 3/gb = C81070/gi = 2521400/ | P | 386.6 | A | 595.6 | P | 605.2 | P |
| | | ug = Mm.98626/len = 579/STRA = rev | | | | | | | |
| 15 | 99778_at | Cluster Incl AU020239: AU020239 Mus musculus cDNA, 3 end/clone = J0529F06/clone_end = 3/gb = AU020239/ | P | 1585.6 | A | 2635.7 | P | 2937.7 | P |
| | | gi = 3375823/ug = Mm 97090/len = 434/STRA = rev | | | | | | | |
| 15 | 101213_at | Cluster Incl X15267 Mouse mRNA for acidic ribosomal phosophoprotein PO/cds = (73,1026)/gb = X15267/gi = 50025/ | P | 1992 | P | 3812.1 | P | 2637.4 | P |
| | | ug = Mm 5286/len = 1094/STRA = for | | | | | | | |
| 15 | AFFX- | X00686 Mouse gene for 18S rRNA (_5, _M, _3 represent transcript regions 5 prime, Middle, | P | 109 | P | 650.3 | P | 433.9 | P |
| | 18SRNA- | and 3 prime respectively) | | | | | | | |
| | Mur/ | | | | | | | | |
| | X00686_{—} | | | | | | | | |
| | 5_at | | | | | | | | |
| 15 | 93520_at | Cluster Incl AF062655 Mus musculus plenty-of-prolines-101 mRNA, complete cds/cds = (23,2716)/gb = AF062655/ | P | 176.1 | P | 313.7 | P | 358.8 | P |
| | | gi = 3153820/ug = Mm 1963/len = 2957/STRA = for | | | | | | | |
| 15 | 97895_f_at | Cluster Incl AW125218: UI-M-BH2 1-aps-c-06-0-UI.s1 Mus musculus cDNA, 3 end/clone = UI-M-BH2.1-aps-c-06-0-UI/ | P | 977.4 | P | 1250.1 | P | 978.1 | A |
| | | clone_end = 3/gb = AW125218/gi = 6100748/ug = Mm 28421/len = 319/STRA = for | | | | | | | |
| 15 | 94557_at | Cluster Incl AI850893 UI-M-BH0-ajt-g-07-0-UI.s1 Mus musculus cDNA, 3 end/clone = UI-M-BH0-ajt-g-07-0-UI/clone_end = 3/ | A | 223.4 | P | 396.9 | P | 338.7 | P |
| | | gb = AI850893/gi = 5494799/ug = Mm 29105/len = 375/STRA = for | | | | | | | |
| 15 | 96311_at | Cluster Incl M11533 Mouse myelin basic protein gene encoding 14 and 18.5 kd proteins/cds = (0,386)/gb = M11533/ | P | 83.3 | P | 242.6 | P | 245.6 | P |
| | | gi = 199045/ug = Mm 2992/len = 387/STRA = for | | | | | | | |
| 15 | 98623_g_at | Cluster Incl X71922 Insulin-like growth factor 2/cds = (0,236)/gb = X71922/gi = 393424/ug = Mm 3862/len = 3301/STRA = for | P | 1587.4 | P | 3110.1 | P | 2766.4 | A |
| 15 | 99648_at | Cluster Incl X62772 Apolipoprotein A-II/cds = (47,355)/gb = X62772/gi = 50016/ug = Mm.43677/len = 473/STRA = for | P | 167.9 | P | 348.9 | A | 250.3 | P |
| 15 | 102854_s_at | Cluster Incl U03434: AT Pase, Cu++ transporting, alpha polypeptide/cds = (82,4557)/gb = U03434/gi = 451486/ug = Mm 14926/ | P | 70.8 | P | 236.1 | P | 214.8 | P |
| | | len = 4889/STRA = for | | | | | | | |
| 15 | 103318_at | Cluster Incl M74516 GA repeat binding protein, beta 1/cds = (132,1280)/gb = M74516/gi = 193384/ug = Mm 1764/len = 2598/ | P | 223 | P | 416.3 | P | 351.9 | A |
| | | STRA = for | | | | | | | |
| 15 | 103391_at | Cluster Incl AI988033: um10h12 x1 Mus musculus cDNA, 3 end/clone = IMAGE-2182727/clone_end = 3/gb = AI988033/ | P | 24.1 | P | 193.6 | P | 92.2 | A |
| | | gi = 5819827/ug = Mm 18654/len = 224/STRA = for | | | | | | | |
| 15 | 94326_r_at | Cluster Incl AW060921 UI-M-BH1-anv-c-12-0-UI s1 Mus musculus cDNA, 3 end/clone = UI-M-BH1-anv-c-12-0-UI/ | P | 375.2 | P | 687.1 | P | 461.1 | P |
| | | clone_end = 3/gb = AW060921/gi = 6008672/ug = Mm 24875/len = 438/STRA = for | | | | | | | |
| 15 | 97996_at | Cluster Incl U58134 Mus musculus poly(A) polymerase VI mRNA, complete cds/cds = (149,1030)/gb = U58134/gi = 1377869/ | P | −6 | A | 52.7 | P | 83.1 | P |
| | | ug = Mm 3170/len = 1046/STRA = for | | | | | | | |
| 15 | 100409_at | Cluster Incl X06340.Cadherin 3/cds = (45,2513)/gb = X06340/gi = 50267/ug = Mm 4658/len = 3187/STRA = for | A | 395.9 | P | 731.2 | P | 688.4 | A |
| 15 | 100879_at | Cluster Incl AF093775 Mus musculus alpha-actinin 3 (Actn3) mRNA, complete cds/cds = (30,2732)/gb = AF093775/ | P | 298.6 | A | 848.2 | P | 742.9 | P |
| | | gi = 3694989/ug = Mm 5316/len = 2855/STRA = for | | | | | | | |
| 15 | 101410_at | Cluster Incl AB000713 Mus musculus mCPE-R mRNA for CPE-receptor, complete cds/cds = (170,802)/gb = AB000713/ | P | 143.5 | A | 550 | P | 542.8 | P |
| | | gi = 2570126/ug = Mm 7339/len = 1783/STRA = for | | | | | | | |
| 15 | 92272_at | Cluster Incl AI060709: ub41h12 r1 Mus musculus cDNA, 5 end/clone = IMAGE-1380359/clone_end = 5/gb = AI060709/ | P | 97.4 | P | 127.8 | A | 126.5 | A |
| | | gi = 3336132/ug = Mm 3609/len = 421/STRA = for | | | | | | | |
| 15 | 92653_at | Cluster Incl AI482432.vg38e09 x1 Mus musculus cDNA, 3 end/clone = IMAGE-863656/clone_end = 3/gb = AI482432/ | P | 40.1 | P | 111.1 | P | 90 | M |
| | | gi = 4375658/ug = Mm 44063/len = 336/STRA = for | | | | | | | |
| 15 | 92756_r_at | Cluster Incl X73580 M musculus mRNA for secretin/cds = (8,409)/gb = X73580/gi = 313710/ug = Mm.4723/len = 490/ | P | 1102.8 | P | 1799.6 | P | 1550.2 | A |
| | | STRA = for | | | | | | | |
| 15 | 93413_at | Cluster Incl AF003000 Telomeric repeat binding factor 2/cds = (65,1552)/gb = AF003000/gi = 2529441/ug = Mm 6402/ | P | 117.8 | P | 162.7 | P | 139.1 | P |
| | | len = 2119/STRA = for | | | | | | | |
| 15 | 93912_at | Cluster Incl AW047616 UI-M-BH1-akm-e-07-0-UI s1 Mus musculus cDNA, 3 end/clone = UI-M-BH1-akm-e-07-0-UI/ | P | 135.3 | A | 232.2 | P | 208.2 | P |
| | | clone_end = 3/gb = AW047616/gi = 5908145/ug = Mm.89571/len = 422/STRA = for | | | | | | | |
| 15 | 94780_at | Cluster Incl AI987985 um10a01 x1 Mus musculus cDNA, 3 end/clone = IMAGE-2182632/clone_end = 3/gb = AI987985/ | P | 117.7 | P | 335.5 | P | 283 | P |
| | | gi = 5819779/ug = Mm 16802/len = 492/STRA = rev | | | | | | | |
| 15 | 99866_at | Cluster Incl AI642417 vt02g03 x1 Mus musculus cDNA, 3 end/clone = IMAGE-1161940/clone_end = 3/gb = AI642417/ | P | 59.7 | P | 202.3 | P | 196.4 | P |
| | | gi = 4720892/ug = Mm.7202/len = 320/STRA = for | | | | | | | |
| 15 | 101755_f_at | Cluster Incl AJ005568: Small proline-rich protein 2J/cds = (0,329)/gb = AJ005568/gi = 3093374/ug = Mm.57084/len = 330/ | P | 1065.5 | P | 1665.6 | P | 1780 | M |
| | | STRA = for | | | | | | | |
| 15 | 101763_at | Cluster Incl AF065145 Mus musculus melatonin-related receptor (Gpr50) mRNA, complete cds/cds = (25,1800)/ | P | 656.4 | P | 1346.7 | P | 1100.4 | A |
| | | gb = AF065145/gi = 3211764/ug = Mm 57095/len = 1801/STRA = for | | | | | | | |
| 15 | 96416_f_at | Cluster Incl Z30939 Histone gene complex 1/cds = (0,176)/gb = Z30939/gi = 496636/ug = Mm 88398/len = 287/STRA = for | P | 807.7 | P | 1220.7 | P | 1189 | A |
| 15 | 101631_at | Cluster Incl AF009414: Mus musculus SOX11 (Sox11) mRNA, complete cds/cds = (288,1475)/gb = AF009414/gi = 2581939/ | P | 146.3 | A | 388.4 | P | 327.1 | P |
| | | ug = Mm 6238/len = 2896/STRA = for | | | | | | | |
| 15 | 95555_at | Cluster Incl AF030169: Mus musculus UNC-119 (Unc-119) mRNA, complete cds/cds = (69,791)/gb = AF030169/gi = 4103939/ | P | 153.1 | P | 193.8 | P | 170.8 | A |
| | | ug = Mm 27744/len = 1196/STRA = for | | | | | | | |
| 15 | 96779_f_at | Cluster Incl AI846315 UI-M-AK1-aez-h-08-0-UI.s1 Mus musculus cDNA, 3 end/clone = UI-M-AK1-aez-h-08-0-UI/ | P | 206.4 | P | 255.8 | P | 294.3 | P |
| | | clone_end = 3/gb = AI846315/gi = 5490221/ug = Mm 29063/len = 345/STRA = for | | | | | | | |
| 15 | 97924_at | Cluster Incl AJ132236 Mus musculus mRNA for UDP-N-acetylglucosamine 2-epimerase/N-acytmannosamine kinase/ | P | 278.2 | P | 548.5 | P | 581.4 | A |
| | | cds = (0,2168)/gb = AJ132236/gi = 4456672/ug = Mm 30193/len = 2169/STRA = for | | | | | | | |
| 15 | 100404_at | Cluster Incl L31959: Transgene insert site 737, Insertional mutation, polycystic kidney disease/cds = (71,2545)/gb = L31959/ | P | 421.1 | P | 695.6 | P | 668.5 | A |
| | | gi = 499646/ug = Mm.4653/len = 3024/STRA = for | | | | | | | |
| 15 | 100923_at | Cluster Incl AJ249706 Mus musculus partial mRNA for myosin X (myo 10 gene)/cds = (0,6092)/gb = AJ249706/gi = 5921504/ | P | 292.7 | P | 432.5 | P | 462.2 | A |
| | | ug = Mm 60590/len = 6093/STRA = for | | | | | | | |
| 15 | 101855_at | Cluster Incl Y14754 Microtubule-associated protein 6/cds = (0,2720)/gb = Y14754/gi = 2769586/ug = Mm 86654/len = 2721/ | P | 31.9 | P | 70.6 | P | 42.8 | P |
| | | STRA = rev | | | | | | | |
| 15 | 103701_at | Cluster Incl AA420331.vc51c06 r1 Mus musculus cDNA, 3 end/clone = IMAGE-778090/clone_end = 3/gb = AA420331/ | A | 221.7 | P | 231.5 | P | 175.2 | P |
| | | gi = 2080888/ug = Mm.24035/len 332/STRA = for | | | | | | | |
| 15 | 103779_at | Cluster Incl AI852829: UI-M-BH0-ajk-b-01-0-UI s1 Mus musculus cDNA, 3 end/clone = UI-M-BH0-ajk-b-01-0-UI/ | A | 303.5 | P | 534.1 | P | 505.6 | A |
| | | clone_end = 3/gb = AI852829/gi = 5496735/ug = Mm 24862/len = 366/STRA = for | | | | | | | |
| 15 | 104439_at | Cluster Incl AI840198: UI-M-AO0-acc-h-05-o-Ul.s1 Mus musculus cDNA, 3 end/clone = UI-M-AO0-acc-h-05-0-UI/ | M | 162.5 | P | 183.8 | P | 224 | P |
| | | clone_end = 3/gb = AI840198/gi = 5474411/ug = Mm.29271/len = 283/STRA = rev | | | | | | | |
| 15 | 104442_at | Cluster Incl AI605538: vo36b03.x1 Mus musculus cDNA 3 end/clone = IMAGE-1051949/clone_end = 3/gb = AI605538/ | P | 104.4 | A | 281.5 | P | 264.4 | P |
| | | gi = 4614705/ug = Mm 29302/len = 286/STRA = rev | | | | | | | |
| 15 | 92399_at | Cluster Incl D26532 Core binding factor alpha 2/cds = (1573,2736)/gb = D26532/gi = 493683/ug = Mm 4081/len = 4295/ | P | 72.9 | A | 161.4 | P | 135.4 | P |
| | | STRA = for | | | | | | | |
| 15 | 92475_g_at | Cluster Incl AF083497 untitled/cds = (31,3255)/gb = AF083497/gi = 3986469/ug = Mm 42128/len = 3924/STRA = for | P | 47.5 | V | 65.9 | M | 51.8 | A |
| 15 | 93464_at | Cluster Incl AI581567 vw82e12.x1 Mus musculus cDNA, 3 end/clone = lMAGE-1261486/clone_end = 3/gb = AI561567/ | P | 128.2 | P | 383 | P | 238.7 | P |
| | | gi = 4512912/ug = Mm 6910/len = 256/STRA = for | | | | | | | |
| 15 | 93466_at | Cluster Incl AF022962 Secretory protein SEC8 (S cerevisiae)/cds = (29,2944)/gb = AF022962/gi = 2547135/ug = Mm 6925/ | P | 41.7 | A | 124.1 | P | 153 | M |
| | | len = 3155/STRA = for | | | | | | | |
| 15 | 95938_at | Cluster Incl AA612212 vo04a01.r1 Mus musculus cDNA, 5 end/clone = IMAGE-1040424/clone_end = 5/gb = AA612212/ | M | 29 | A | 143.1 | P | 113.2 | P |
| | | gi = 2460890/ug = Mm.24840/len = 357/STRA = for | | | | | | | |
| 15 | 96513_at | Cluster Incl AA794350 vr46a11 s1 Mus musculus cDNA, 5 end/clone = IMAGE-1123676/clone_end = 5/gb = AA794350/ | P | 49.6 | A | 253.3 | P | 156.3 | P |
| | | gi = 2857305/ug = Mm.25583/len = 109/STRA = rev | | | | | | | |
| 15 | 97744_at | Cluster Incl AI847619 UI-M-AP1-agg-d-12-0-UI s1 Mus musculus cDNA, 3 end/clone = UI-M-AP1-agg-d-12-0-UI/ | A | 316.9 | P | 507.9 | P | 572.1 | P |
| | | clone_end = 3/gb = AI847619/gi = 5491525/ug = Mm 40174/len = 364/STRA = rev | | | | | | | |
| 15 | 102152_f_at | Cluster Incl M16724 Mouse Ig germline H-chain gene V-region (subfamily T15), exons 1 and 2/cds = (0,187)/gb = M16724/ | P | 318.1 | P | 460.8 | P | 469.7 | M |
| | | gi = 196262/ug = Mm 46800/len = 188/STRA = for | | | | | | | |
| 15 | AFFX- | X00686 Mouse gene for 18S rRNA (_5, _M, _3 represent transcript regions 5 prime, Middle, | A | 344.6 | M | 663.6 | P | 427.1 | P |
| | 18SRNA- | and 3 prime respectively) | | | | | | | |
| | Mur/ | | | | | | | | |
| | X00686_{—} | | | | | | | | |
| | 3_at | | | | | | | | |
| 15 | 93087_r_at | Cluster lncl AA267185 mz92g02 r1 Mus musculus cDNA, 5 end/clone = IMAGE-720914/clone_end = 5/gb = AA267185/ | P | 304.9 | P | 464.5 | P | 434.6 | P |
| | | gi = 1903945/ug = Mm 16253/len = 229/STRA = for | | | | | | | |
| 15 | 95138_at | Cluster Incl AI836568 UI-M-AP0-abf-a-06-0-UI s1 Mus musculus cDNA, 3 end/clone = UI-M-AP0-abf-a-06-0-UI/ | A | 154.4 | P | 282.2 | P | 220 | P |
| | | clone_end = 3/gb = AI836568/gi = 5470781/ug = Mm 29432/len = 285/STRA = for | | | | | | | |
| 15 | 104109_at | Cluster Incl AI853773 UI-M-BH0-ajg-e-03-0-UI s1 Mus musculus cDNA, 3 end/clone = UI-M-BH0-ajg-e-03-0-UI/ | A | 129.5 | P | 164.8 | A | 164.8 | M |
| | | clone_end = 3/gb = AI853773/gi = 5497679/ug = Mm 21912/len = 453/STRA = for | | | | | | | |
| 15 | 104590_at | Cluster Incl L13171 Myocyte enhancer factor 2C/cds = UNKNOWN/gb = L13171/gi = 293728/ug = Mm 24001/len = 1656/ | A | 148.6 | A | 383.9 | P | 532.3 | P |
| | | STRA = for | | | | | | | |
| 15 | 97355_at | Cluster Incl AW050020 UI-M-BH1-amm-a-08-0-UI s1 Mus musculus cDNA, 3 end/clone = UI-M-BH1-amm-a-08-0-UI/ | P | 57.7 | P | 121.7 | A | 133 | A |
| | | clone_end = 3/gb = AW050020/gi = 5910549/ug = Mm 29632/len = 458/STRA = for | | | | | | | |
| 15 | 97975_at | Cluster Incl D85612 Nuclear factor of activated T-cells, cytoplasmic 3/cds = (7,3234)/gb = D85612/gi = 1906311/ | P | 189.6 | P | 474.7 | A | 201.3 | A |
| | | ug = Mm 3107/len = 3638/STRA = for | | | | | | | |
| 15 | 103460_at | Cluster Incl AI849939: UI-M-BG0-ahz-g-03-0-UI s1 Mus musculus cDNA, 3 end/clone = UI-M-BG0-ahz-g-03-0-UI/ | P | 227.2 | A | 339.2 | P | 425.1 | P |
| | | clone_end = 3/gb = AI849939/gi = 5493845/ug = Mm 21697/len = 440/STRA = for | | | | | | | |
| 15 | 103925_at | Cluster Incl AW120605 UI-M-BH2.3-anx-f-01-0-UI.s1 Mus musculus cDNA, 3 end/clone = UI-M-BH2 3-anx-f-01-0-UI/ | P | 189.2 | P | 353 | P | 388.6 | A |
| | | clone_end = 3/gb = AW120605/gi = 6095938/ug = Mm 25080/len = 151/STRA = for | | | | | | | |
| 15 | 94104_at | Cluster Incl AF051911 Telomerase reverse transcriptase/cds = (29,3397)/gb = AF051911/gi = 3005591/ug= Mm 10109/ | P | 205.4 | P | 259 | M | 264.7 | M |
| | | len = 3426/STRA = for | | | | | | | |
| 15 | 98303_at | Cluster Incl X79131: M. musculus mRNA for IB3/5-polypeptide/cds = (833,2869)/gb = X79131/gi = 485857/ug = Mm 42143/ | A | 306.5 | P | 374.4 | P | 327.7 | M |
| | | len = 3008/STRA = for | | | | | | | |
| 15 | 99439_at | Cluster Incl X67735.MAS1 oncogene/cds = (0,974)/gb = X67735/gi = 53011/ug = Mm 57182/len = 975/STRA = for | P | 286.6 | P | 461 | P | 437.4 | P |
| 15 | 95869_at | Cluster Incl C78041 C78041 Mus musculus cDNA, 3 end/clone = J0041H05/clone_end = 3/gb = C78041/gi = 2518371/ | P | 5.9 | A | 64.8 | P | 73.4 | P |
| | | ug = Mm.24980/len = 538/STRA = rev | | | | | | | |
| 15 | 95879_at | Cluster Incl C79052 C79052 Mus musculus cDNA, 3 end/clone = J0059F07/clone_end = 3/gb = C79052/gi = 2519382/ | P | 8.2 | A | 49.8 | A | 76 | P |
| | | ug = Mm 25008/len = 608/STRA = rev | | | | | | | |
| 15 | 100686_at | Cluster Incl M20632 Mouse LLRep3 protein mRNA from a repetitive element, complete cds/cds = (1022,1687)/gb = M20632/ | P | 1523.6 | A | 3754.3 | P | 2979.2 | P |
| | | gi = 200811/ug = Mm 1129/len = 1728/STRA = for | | | | | | | |
| 15 | AFFX- | X00686 Mouse gene for 18S rRNA (_5, _M, _3 represent transcript regions 5 prime, Middle, | A | 67 | A | 443.8 | P | 180.5 | P |
| | 18SRNA- | and 3 prime respectively) | | | | | | | |
| | Mur/ | | | | | | | | |
| | X00686_{—} | | | | | | | | |
| | M_at | | | | | | | | |
| 15 | 99980_at | Cluster Incl M35986 Mouse homeobox-6 1 protein (Hox-6.1) mRNA, 3 end/cds = (0,317)/gb = M35986/gi = 193981/ | P | 206.7 | P | 315.8 | P | 251.1 | P |
| | | ug = Mm 4444/len = 318/STRA = for | | | | | | | |
| 15 | 102695_at | Cluster Incl AF021335 Mus musculus T cell receptor gamma locus, TCR gamma 2 and gamma 4 gene clusters/ | P | 189.1 | M | 337.4 | P | 344 | A |
| | | cds = (0,571)/gb = AF021335/gi = 2444469/ug = Mm 14145/len = 572/STRA = for | | | | | | | |
| 15 | 102865_at | Cluster Incl U58993 MAD homolog 5 (Drosophila)/cds = (266,1663)/gb = U58993/gi = 1518646/ug = Mm.1470/len = 2185/ | P | 90.6 | M | 170.2 | P | 152.7 | P |
| | | STRA= for | | | | | | | |
| 15 | 102871_at | Cluster Incl L77867 Mus musculus MEP mRNA, complete cds/cds = (202,3246)/gb = L77867/gi = 1911182/ug = Mm.1480/ | P | 82 | P | 335.4 | A | 130.5 | A |
| | | len = 3464/STRA = for | | | | | | | |
| 15 | 103229_at | Cluster Incl U64828 Mus musculus steroid receptor coactivator-1 (mSRC-1) mRNA, complete cds/cds = (625,4842)/ | P | 132.5 | A | 346.2 | P | 219.9 | P |
| | | gb = U64828/gi = 1490875/ug = Mm 2028/1en = 5169/STRA = for | | | | | | | |
| 15 | 104010_at | Cluster Incl U62906: Mus musculus zinc finger protein 94 (Zfp94) mRNA, complete cds/cds = (180,1742)/gb = U62906/ | A | 83.4 | P | 175.7 | P | 133.2 | P |
| | | gi = 4097496/ug = Mm.26782/len = 1997/STRA = for | | | | | | | |
| 15 | 92364_at | Cluster Incl AB028499 Mus musculus mRNA for Flamingo 1, complete cds/cds = (0,8762)/gb = AB028499/gi = 5832710/ | P | 445 | A | 926.4 | P | 629.7 | P |
| | | ug = Mm 39728/len = 8950/STRA = for | | | | | | | |
| 15 | 93900_at | Cluster Incl AW050268 UI-M-BH1-ans-d-03-0-UI.s1 Mus musculus cDNA, 3 end/clone = UI-M-BH1-ans-d-03-0-UI/ | P | 406.2 | P | 549 | A | 668.2 | A |
| | | clone_end = 3/gb = AW050268/gi = 5910797/ug = Mm 89316/len = 221/STRA = for | | | | | | | |
| 15 | 95284_at | Cluster Incl AI662509 mt33c08 x1 Mus musculus cDNA, 3 end/clone = IMAGE-822862/clone_end = 3/gb = AI662509/ | P | 67.9 | P | 103.3 | A | 101.9 | M |
| | | gi = 4766092/ug = Mm.1850/len = 231/STRA = for | | | | | | | |
| 15 | 95995_at | Cluster Incl C80108 C80108 Mus musculus cDNA, 3 end/clone = J0076F01/clone_end = 3/gb = C80108/gi = 2520438/ | A | 619 | A | 255.9 | P | 276.8 | P |
| | | ug = Mm 25061/1en = 349/STRA = rev | | | | | | | |
| 15 | 100282_at | Cluster Incl Z23107 5-hydroxytryptamine (serotonin) receptor 7/cds = (108,1454)/gb = Z23107/gi = 396586/ug = Mm 8053/ | A | 156.2 | A | 355.5 | P | 396.3 | P |
| | | len = 1540/STRA = for | | | | | | | |
| 15 | 95872_at | Cluster Incl C78535: C78535 Mus musculus cDNA, 3 end/clone = J0050H08/clone_end = 3/gb = C78535/gi = 2518865/ | P | 29.4 | A | 103.6 | P | 106.4 | A |
| | | ug= Mm.24993/len = 576/STRA = rev | | | | | | | |
| 15 | 101159_at | Cluster Incl Z23066: Microphthalmia-associated transcription factor/cds = (129,1388)/gb = Z23066/gi = 396739/ug = Mm 4978/ | M | 105.3 | A | 239.4 | P | 168 | P |
| | | len = 1922/STRA = for | | | | | | | |
| 15 | 101618_r_at | Cluster Incl U59758 Mus musculus p53-variant (p53) mRNA, partial cds/cds = (0,161)/gb= U59758/gi = 1399879/ | A | 291.2 | A | 480.3 | P | 330.3 | A |
| | | ug = Mm.6186/len = 846/STRA = for | | | | | | | |
| 15 | 103348_at | Cluster Incl AI837688 UI-M-AK0-adj-c-07-0-UI.s1 Mus musculus cDNA, 3 end/clone = UI-M-AK0-adj-c-07-0-UI/ | A | 135.6 | P | 484.3 | A | 453.8 | P |
| | | clone_end = 3/gb = AI837688/gi = 5471901/ug = Mm 18311/len = 392/STRA = for | | | | | | | |
| 15 | 98507_at | Cluster Incl AI834950 UI-M-AM1-afw-b-05-0-UI.s1 Mus musculus cDNA, 3 end/clone = UI-M-AM1-afw-b-05-0-UI/ | A | 142.2 | M | 188.8 | P | 177.4 | A |
| | | clone_end = 3/gb = AI834950/gi = 5469163/ug = Mm 35822/len = 315/STRA = rev | | | | | | | |
| 15 | 100021_at | Cluster Incl M17640 Mus musculus acetylcholine receptor alpha-subunit mRNA, complete cds/cds = (104,1477)/ | A | 85.2 | A | 151.3 | M | 239.7 | M |
| | | gb = M17640/gi = 2073542/ug = Mm 4583/len = 1860/STRA = for | | | | | | | |
| 15 | 101381_at | Cluster Incl D86066 Mus musculus mRNA for mouse rabaptin-5, complete cds/cds = (50,2638)/gb = D86066/gi = 2329848/ | A | 61.8 | P | 115.2 | A | 112.1 | A |
| | | ug = Mm 7087/len = 3095/STRA = for | | | | | | | |
| 15 | 104195_at | Cluster Incl AA939440: vz49d01.r1 Mus musculus cDNA, 5 end/clone = IMAGE-1329793/clone_end = 5/gb = AA939440/ | A | 232.6 | A | 261.8 | P | 268.1 | P |
| | | gi = 3100217/ug = Mm 27503/len = 379/STRA = for | | | | | | | |
| 15 | 104415_at | Cluster Incl AA833293 ud05d05 r1 Mus musculus cDNA, 5 end/clone = IMAGE-1434249/clone_end = 5/gb= AA833293/ | P | 67.4 | M | 228.2 | P | 165.1 | A |
| | | gi = 2907021/ug = Mm 28907/len = 548/STRA = for | | | | | | | |
| 15 | 93155_at | Cluster Incl AI847069 UI-M-AP1-agn-c-04-0-UI s1 Mus musculus cDNA, 3 end/clone = UI-M-AP1-agn-c-04-0-UI/ | A | 25.7 | A | 68.2 | P | 36.2 | P |
| | | clone_end = 3/gb = AI847069/gi = 5490975/ug = Mm.5263/len = 263/STRA = for | | | | | | | |
| 15 | 97711_at | Cluster Incl AI606967 vw37a01 x1 Mus musculus cDNA, 3 end/clone = IMAGE-1245960/clone_end = 3/gb = AI606967/ | A | 105.4 | A | 189.9 | P | 130.1 | P |
| | | gi = 4616134/ug = Mm 38364/len = 387/STRA = rev | | | | | | | |
| 15 | 99908_at | Cluster Incl AB010557 Mus musculus mRNA for Pax4, complete cds/cds = (206,1255)/gb = AB010557/gi = 2809092/ | P | 305.2 | P | 585.1 | P | 521.3 | A |
| | | ug = Mm 8026/len = 1295/STRA = for | | | | | | | |
| 15 | 100749_at | Cluster Incl AA414976: vc49e02 r1 Mus musculus cDNA, 3 end/clone = IMAGE-777914/clone_end = 3/gb = AA414976/ | P | 111 | A | 256.7 | P | 183.9 | A |
| | | gi = 2075103/ug = Mm 3802/len = 332/STRA = rev | | | | | | | |
| 16 | 92567_at | Cluster Incl L02918 Mouse procollagen type V alpha 2 (Col5a-2) mRNA, complete cds/cds = (0,4493)/gb = L02918/ | P | 2400.6 | P | 1699.1 | P | 2889.4 | P |
| | | gi = 309180/ug = Mm 10299/len = 4494/STRA = for | | | | | | | |
| 16 | 92587_at | Cluster Incl L29123 Mus musculus adrenodoxin mRNA, complete cds/cds = (88,654)/gb = L29123/gi = 457297/ug = Mm 1061/ | P | 711.7 | P | 592.2 | P | 681 | P |
| | | len = 883/STRA = for | | | | | | | |
| 16 | 92795_at | Cluster Incl M72414 Microtubule-associated protein 4/cds = (490,3867)/gb = M72414/gi = 456554/ug = Mm 12625/len = 6082/ | P | 498.5 | P | 476.8 | P | 554.6 | P |
| | | STRA = for | | | | | | | |
| 16 | 92816_r_at | Cluster Incl X03039.Eukaryotic translation Initiation factor 4A1/cds = UNKNOWN/gb = X03039/gi = 50812/ug = Mm 12858/ | P | 1847.3 | P | 1814.1 | P | 2273.4 | P |
| | | len = 1407/STRA = for | | | | | | | |
| 16 | 92833_at | Cluster Incl L07645 Histidine ammonia tyase/cds = UNKNOWN/gb= L07645/gi = 193751/ug = Mm 13000/len = 2281/ | P | 1607.6 | P | 1734.5 | P | 2938.4 | P |
| | | STRA = for | | | | | | | |
| 16 | 93029_at | Cluster Incl U68564 Isocitrale dehydrogenase 3 (NAD+), gamma/cds = (42,1223)/gb = U68564/gi = 1546828/ug = Mm 14825/ | P | 1101 | P | 858.7 | P | 1014.5 | P |
| | | len = 1335/STRA = for | | | | | | | |
| 16 | 93316_at | Cluster Incl AB017026 Mus musculus mRNA for oxysterol-binding protein, complete cds/cds = (827,2059)/gb = AB017026/ | P | 616.7 | P | 431.3 | P | 602.9 | P |
| | | gi = 3551522/ug = Mm 18502/len = 2931/STRA = for | | | | | | | |
| 16 | 93333_at | Cluster Incl U05333 Mus musculus co-chaperonin cofactor A mRNA, complete cds/cds = (7,333)/gb = U05333/gi = 1117784/ | P | 596.7 | P | 529.3 | P | 585.7 | P |
| | | ug = Mm 1863/len = 508/STRA = for | | | | | | | |
| 16 | 93571_at | Cluster Incl M74773 Beta-spectrin 2, non-erythrocytic/cds = (366,7457)/gb = M74773/gi = 598330/ug = Mm 20405/len = 7524/ | P | 1935.9 | P | 1432.9 | P | 2247.7 | P |
| | | STRA = for | | | | | | | |
| 16 | 93574_at | Cluster Incl AF036164 Mus musculus pigment epithelium-derived factor (PEDF) mRNA, complete cds/cds = (33,1286)/ | P | 465.5 | P | 394.9 | P | 621 | P |
| | | gb = AF036164/gi = 3808220/ug = Mm 2044/len = 1648/STRA = for | | | | | | | |
| 16 | 93731_at | Cluster Incl AF090334 Mus musculus FK506 binding protein (Fkbp63) mRNA, complete cds/cds = (74,1786)/gb = AF090334/ | P | 1143.2 | P | 919.4 | P | 1426 | P |
| | | gi = 3860028/ug = Mm 20943/len = 2945/STRA = for | | | | | | | |
| 16 | 93750_at | Cluster Incl J04953 Gelsolin/cds = (18,2213)/gb = J04953/gi = 193463/ug = Mm 21109/len = 2438/STRA = for | P | 2058.9 | P | 1587.9 | P | 3110.4 | P |
| 16 | 93754_at | Cluster Incl AF030343 Mus musculus peroxisomal/mitochondrial dienoyl-CoA isomerase ECH1p (Ech1) mRNA, complete | P | 1151.7 | P | 724.1 | P | 975.5 | P |
| | | cds/cds = (23,1006)/gb = AF030343/gi = 2606085/ug = Mm 2112/len = 1138/STRA = for | | | | | | | |
| 16 | 94031_at | Cluster Incl X95403 M musculus mRNA for GTP-binding protein/cds = (207,845)/gb = X95403/gi = 1177546/ug = Mm.21905/ | P | 1190.1 | P | 997.5 | P | 1246.1 | P |
| | | len = 1008/STRA = for | | | | | | | |
| 16 | 94059_at | Cluster Incl AI852524 UI-M-BH0-aiw-d-04-0-UI.s1 Mus musculus cDNA, 3 end/clone = UI-M-BH0-aiw-d-04-0-UI/ | P | 939.1 | P | 346.8 | P | 922.9 | P |
| | | clone_end = 3/gb = AI852524/gi = 5496430/ug = Mm 22029/len = 382/STRA = for | | | | | | | |
| 16 | 94233_at | Cluster Incl AW048642 UI-M-BH1-amd-d-06-0-UI s1 Mus musculus cDNA, 3 end/clone = UI-M-BH1-amd-d-06-0-UI/ | P | 252.2 | P | 202 | P | 300.9 | P |
| | | clone_end = 3/gb = AW048642/gi = 5909171/ug = Mm.22294/len = 480/STRA = for | | | | | | | |
| 16 | 94269_at | Cluster Incl L40934 Mus musculus rab6/rab5-associated protein (rab6) mRNA, partial cds/cds = (0,307)/gb = L40934/ | P | 1840.2 | P | 1965.5 | P | 2756.6 | P |
| | | gi = 722666/ug = Mm 22473/len = 308/STRA = for | | | | | | | |
| 16 | 94284_at | Cluster Incl AW122731 UI-M-BH2 2-aot-e-08-0-UI s1 Mus musculus cDNA, 3 end/clone = UI-M-BH2 2-aot-e-08-0-UI/ | P | 863.7 | P | 774.8 | P | 929.8 | P |
| | | clone_end = 3/gb = AW122731/gi = 6098261/ug = Mm 22560/len = 204/STRA = for | | | | | | | |
| 16 | 94289_r_at | Cluster Incl AI851574 UI-M-BH0-aki-f-10-0-UI s1 Mus musculus cDNA, 3 end/clone = UI-M-BH0-aki-f-10-0-UI/clone_end = 3/ | P | 624.4 | P | 570.1 | P | 964.1 | P |
| | | gb = AI851574/gi = 5495480/ug = Mm 22575/len = 459/STRA = for | | | | | | | |
| 16 | 94870_f_at | Cluster Incl AW124226 UI-M-BH2 1-aph-d-10-0-UI.s1 Mus musculus cDNA, 3 end/clone = UI-M-BH2.1-aph-d-10-0-UI/ | P | 385.6 | P | 232.1 | P | 323.4 | P |
| | | clone_end = 3/gb = AW124226/gi = 6099756/ug = Mm 23755/len = 382/STRA = for | | | | | | | |
| 16 | 94872_at | Cluster Incl Y08135 M musculus mRNA for ASM-like phosphodiesterase 3a/cds = (92,1429)/gb = Y08135/gi = 1552349/ | P | 368.8 | P | 338.7 | P | 509.1 | P |
| | | ug = Mm 2379/len = 1758/STRA = for | | | | | | | |
| 16 | 94878_at | Cluster Incl AW125224 UI-M-BH2 1-aps-d-02-0-UI s1 Mus musculus cDNA, 3 end/clone = UI-M-BH2 1-aps-d-02-0-UI/ | P | 594.7 | P | 634.1 | P | 735.6 | P |
| | | clone_end = 3/gb = AW125224/gi = 6100754/ug = Mm 23839/len = 356/STRA = for | | | | | | | |
| 16 | 94889_at | Cluster Incl AW121235 UI-M-BH2 3-aok-f-05-0-UI s1 Mus musculus cDNA, 3 end/clone = UI-M-BH2 3-aok-f-05-0-UI/ | P | 1861 | P | 1506.3 | P | 2220.9 | P |
| | | clone_end = 3/gb = AW121235/gi = 6096568/ug = Mm 23943/len = 293/STRA = for | | | | | | | |
| 16 | 94892_r_at | Cluster Incl M27938: Male enhanced antigen 1/cds = (80,604)/gb = M27938/gi = 199115/ug = Mm.2395/len = 841/STRA = for | P | 791.3 | P | 973.8 | P | 1230.2 | P |
| 16 | 94899_at | Cluster Incl U73200. Mus musculus p116Rip mRNA, complete cds/cds = (191,3265)/gb = U73200/gi = 1657836/ug = Mm 2402/ | P | 1711.7 | P | 1934.1 | P | 2093.7 | P |
| | | len = 4010/STRA = for | | | | | | | |
| 16 | 95411_at | Cluster Incl AB025407 Mus musculus mRNA for sid2057p, complete cds/cds = (83,628)/gb = AB025407/gi = 5931562/ | P | 1333.5 | P | 1202.8 | P | 1327.1 | P |
| | | ug = Mm 24231/len = 1400/STRA = for | | | | | | | |
| 16 | 96049_at | Cluster Incl X53928: M musculus mRNA for PGI (biglycan)/cds = (234,1343)/gb = X53928/gi = 53666/ug = Mm 2608/ | P | 2755.3 | P | 2505.7 | P | 3711.5 | P |
| | | len = 1964/STRA = for | | | | | | | |
| 16 | 96629_at | Cluster Incl X04097: Mouse kidney lestosterone-regulated RP2 mRNA/cds = (41,1114)/gb = X04097/gi = 54003/ | P | 433.9 | P | 370.3 | P | 381.9 | P |
| | | ug = Mm.27194/len = 2005/STRA = for | | | | | | | |
| 16 | 96634_at | Cluster Incl AI850090: UI-M-BG0-aia-g-01-0-UI.s1 Mus musculus cDNA, 3 end/clone = UI-M-BG0-aia-g-01-0-UI/ | P | 393 | P | 301.9 | P | 343 | P |
| | | clone_end = 3/gb = AI850090/gi = 5493996/ug = Mm 27227/len = 481/STRA = for | | | | | | | |
| 16 | 96641_at | Cluster Incl AW060674 UI-M-BH1-anl-d-04-0-UI s1 Mus musculus cDNA, 3 end/clone = UI-M-BH1-anl-d-04-0-UI/ | P | 542.3 | P | 561.3 | P | 599.5 | P |
| | | clone_end = 3/gb = AW060674/gi = 6008425/ug = Mm 27285/len = 486/STRA = for | | | | | | | |
| 16 | 96708_at | Cluster Incl AW120643 UI-M-BH2 3-anz-f-05-0-UI.s1 Mus musculus cDNA, 3 end/clone = UI-M-BH2 3-anz-f-05-0-UI/ | P | 1096.9 | P | 1119.8 | P | 1385.4 | P |
| | | clone_end = 3/gb = AW120643/gi = 6096016/ug = Mm 27606/len = 505/STRA = for | | | | | | | |
| 16 | 97277_at | Cluster Incl AI844179 UI-M-AL1-ahk-a-04-0-UI s1 Mus musculus cDNA, 3 end/clone = UI-M-AL1-ahk-a-04-0-UI/ | P | 347 | P | 243.6 | P | 360.8 | P |
| | | clone_end = 3/gb = AI844179/gi = 5488085/ug = Mm 27951/len = 281/STRA = for | | | | | | | |
| 16 | 97841_at | Cluster Incl AI838663 UI-M-AO0-aca-d-07-0-UI.s1 Mus musculus cDNA, 3 end/clone = UI-M-AO0-aca-d-07-0-UI/ | P | 1106.6 | P | 1051.2 | P | 1388 | P |
| | | clone_end = 3/gb = AI838663/gi = 5472876/ug = Mm 28258/len = 368/STRA = for | | | | | | | |
| 16 | 97901_at | Cluster Incl X60831 Mouse ubf gene for transcription factor UBF/cds = UNKNOWN/gb = X60831/gi = 55115/ug = Mm 2845/ | P | 417.9 | P | 476 | P | 566.4 | P |
| | | len = 3031/STRA = for | | | | | | | |
| 16 | 94454_at | Cluster Incl AF085348 Mus musculus proline-nch protein (Bprp) mRNA, complete cds/cds = (15,521)/gb = AF085348/ | P | 1107.4 | P | 849.3 | P | 1098.6 | P |
| | | gi = 3549610/ug = Mm 28797/len = 1779/STRA = for | | | | | | | |
| 16 | 94531_at | Cluster Incl AW124582: UI-M-BH2 1-apt-e-11-0-UI s1 Mus musculus cDNA, 3 end/clone = UI-M-BH2 1-apt-e-11-0-UI/ | P | 451.4 | P | 305.9 | P | 420 | P |
| | | clone_end = 3/gb = AW124582/gi = 6100112/ug = Mm 29042/len = 360/STRA = for | | | | | | | |
| 16 | 94549_at | Cluster Incl AI315650: uj31d03 x1 Mus musculus cDNA, 3 end/clone = IMAGE-1921541/clone_end = 3/gb = AI315650/ | P | 198.8 | P | 38.8 | P | 168.9 | P |
| | | gi = 4030917/ug = Mm 29078/len = 514/STRA = for | | | | | | | |
| 16 | 95054_at | Cluster Incl AI849620: UI-M-AH1-agr-h-02-0-UI s1 Mus musculus cDNA, 3 end/clone = UI-M-AH1-agr-h-02-0-UI/ | P | 473.3 | P | 424 | P | 528.3 | P |
| | | clone_end = 3/gb = AI849620/gi = 5493526/ug = Mm 29144/len = 340/STRA = for | | | | | | | |
| 16 | 95074_at | Cluster Incl AW125309: UI-M-BH2.1-apy-d-05-0-UI s1 Mus musculus cDNA, 3 end/clone = UI-M-BH2 1-apy-d-05-0-UI/ | P | 359.4 | P | 303.5 | P | 430.6 | P |
| | | clone_end = 3/gb = AW125309/gi = 6100839/ug = Mm 29198/len = 276/STRA = for | | | | | | | |
| 16 | 95136_at | Cluster Incl AW049185: UI-M-BH1-amq-g-01-0-UI.s1 Mus musculus cDNA, 3 end/clone = UI-M-BH1-amq-g-01-0-UI/ | P | 379 | P | 468.5 | P | 652.5 | P |
| | | clone_end = 3/gb = AW049185/gi = 5909714/ug = Mm 29430/len = 483/STRA = for | | | | | | | |
| 16 | 95701_at | Cluster Incl AW124069: UI-M-BH2.1-apn-f-11-0-UI.s1 Mus musculus cDNA, 3 end/clone = UI-M-BH2 1-apn-f-11-0-UI/ | P | 192.6 | P | 207.4 | P | 270.9 | P |
| | | clone_end = 3/gb = AW124069/gi = 6099599/ug = Mm 29690/len = 390/STRA = for | | | | | | | |
| 16 | 95732_at | Cluster Incl AW047746: UI-M-BH1-alk-b-11-0-UI s1 Mus musculus cDNA, 3 end/clone = UI-M-BH1-alk-b-11-0-UI/ | P | 649.4 | P | 737 | P | 721 | P |
| | | clone_end = 3/gb = AW047746/gi = 5908275/ug = Mm 29743/len = 406/STRA = for | | | | | | | |
| 16 | 95737_at | Cluster Incl AW120814: UI-M-BH2.3-aoa-b-07-0-UI s1 Mus musculus cDNA, 3 end/clone = UI-M-BH2 3-aoa-b-07-0-UI/ | P | 790.5 | P | 760.9 | P | 940.3 | P |
| | | clone_end = 3/gb = AW120814/gi = 6096147/ug = Mm 29750/len = 314/STRA = for | | | | | | | |
| 16 | 96338_at | Cluster Incl AW125059: UI-M-BH2.1-apx-c-07-0-UI s1 Mus musculus cDNA, 3 end/clone = UI-M-BH2 1-apx-c-07-0-UI/ | P | 938.2 | P | 749.5 | P | 1142 | P |
| | | clone_end = 3/gb = AW125059/gi = 6100589/ug = Mm 29978/len = 448/STRA = for | | | | | | | |
| 16 | 96845_at | Cluster Incl AI849577 UI-M-AH1-agr-d-03-0-UI.s1 Mus musculus cDNA, 3 end/clone = UI-M-AH1-agr-d-03-0-UI/ | P | 284.7 | P | 219 | P | 264.2 | P |
| | | clone_end = 3/gb = AI849577/gi = 5493483/ug = Mm 30023/len = 242/STRA = for | | | | | | | |
| 16 | 96894_at | Cluster Incl AW122685 UI-M-BH2.2-aot-a-06-0-UI s1 Mus musculus cDNA, 3 end/clone = UI-M-BH2 2-aot-a-06-0-UI/ | P | 423.2 | P | 412.2 | P | 492.6 | P |
| | | clone_end = 3/gb = AW122685/gi = 6098215/ug = Mm 30103/len = 445/STRA = for | | | | | | | |
| 16 | 96934_at | Cluster Incl AW049074 UI-M-BH1-amt-d-01-0-UI s1 Mus musculus cDNA, 3 end/clone = UI-M-BH1-aml-d-01-0-UI/ | P | 902.6 | P | 1054.4 | P | 1284.5 | P |
| | | clone_end = 3/gb = AW049074/gi = 5909603/ug = Mm 30179/len = 285/STRA = for | | | | | | | |
| 16 | 97463_g_at | Cluster Incl AI847584 UI-M-AP1-agg-a-06-0-UI s1 Mus musculus cDNA, 3 end/clone = UI-M-AP1-agg-a-06-0-UI/ | P | 2410.4 | P | 2266.3 | P | 2812.5 | P |
| | | clone_end = 3/gb = AI847584/gi = 5491490/ug = Mm 3035/len = 340/STRA = for | | | | | | | |
| 16 | 98047_at | Cluster Incl AI851583 UI-M-BH0-aki-g-07-0-UI s1 Mus musculus cDNA, 3 end/clone = UI-M-BH0-aki-g-07-0-UI/ | P | 384 | P | 347.9 | P | 433.1 | P |
| | | clone_end = 3/gb = AI851583/gi = 5495489/ug = Mm.34261/len = 460/STRA = for | | | | | | | |
| 16 | 98070_at | Cluster Incl AI844794 UI-M-AH1-agx-a-02-0-UI s1 Mus musculus cDNA, 3 end/clone = UI-M-AH1-agx-a-02-0-UI/ | P | 867.3 | P | 832.6 | P | 1037.1 | P |
| | | clone_end = 3/gb = AI844794/gi = 5488700/ug = Mm 34456/len = 394/STRA = for | | | | | | | |
| 16 | 98522_at | Cluster Incl AI839158 UI-M-AO0-ach-f-03-0-UI s1 Mus musculus cDNA, 3 end/clone = UI-M-AO0-ach-f-03-0-UI/ | P | 1176.4 | P | 1177.2 | P | 1329.2 | P |
| | | clone_end = 3/gb = AI839158/gi = 5473371/ug = Mm 35812/len = 419/STRA = for | | | | | | | |
| 16 | 98528_at | Cluster Incl AI854901 UI-M-BH0-akb-h-06-0-UI s1 Mus musculus cDNA, 3 end/clone = UI-M-BH0-akb-h-06-0-UI/ | P | 1219 | P | 1216.6 | P | 1516.3 | P |
| | | clone_end = 3/gb = AI854901/gi = 5498807/ug = Mm 35837/len = 398/STRA = for | | | | | | | |
| 16 | 98625_s_at | Cluster Incl M84147 Mouse alcohol dehydrogenase-B2 (Adh-2) mRNA, putative cds/cds = (39,1163)/gb = M84147/ | P | 1000.7 | P | 728.4 | P | 849.3 | P |
| | | gi = 191721/ug = Mm.3874/len = 1360/STRA = for | | | | | | | |
| 16 | 98633_at | Cluster Incl AI854863 UI-M-BH0-akb-d-12-0-UI s1 Mus musculus cDNA, 3 end/clone = UI-M-BH0-akb-d-12-0-UI/ | P | 559.8 | P | 538.5 | P | 1128.4 | P |
| | | clone_end = 3/gb = AI854863/gi = 5498769/ug = Mm 38801/len = 419/STRA = for | | | | | | | |
| 16 | 98635_at | Cluster Incl AI854629 UI-M-BH0-ake-e-11-0-UI s1 Mus musculus cDNA, 3 end/clone = UI-M-BH0-ake-e-11-0-UI/ | P | 533.6 | P | 495.9 | P | 545.8 | P |
| | | clone_end = 3/gb = AI854629/gi = 5498535/ug = Mm 38802/len = 492/STRA = for | | | | | | | |
| 16 | 99176_at | Cluster Incl AI843393: UI-M-AQ1-adz-g-04-0-UI s1 Mus musculus cDNA, 3 end/clone = UI-M-AQ1-adz-g-04-0-UI/ | P | 458 | P | 327.5 | P | 438.6 | P |
| | | clone_end = 3/gb = AI843393/gi = 5477606/ug = Mm 41785/len = 394/STRA = for | | | | | | | |
| 16 | 99191_at | Cluster Incl AI844939 UI-M-BG0-ahv-g-09-0-UI s1 Mus musculus cDNA, 3 end/clone = UI-M-BG0-ahv-g-09-0-UI/ | P | 1468.5 | P | 1136 | P | 1975.2 | P |
| | | clone_end = 3/gb = AI844939/gi = 5488845/ug = Mm 41906/len = 413/STRA = for | | | | | | | |
| 16 | 99617_at | Cluster Incl AI846299: UI-M-AK1-aez-f-12-0-UI s1 Mus musculus cDNA, 3 end/clone = UI-M-AK1-aez-f-12-0-UI/ | P | 1108.3 | P | 1069.2 | P | 1172.8 | P |
| | | clone_end = 3/gb = AI846299/gi = 5490205/ug = Mm 43040/len = 335/STRA = for | | | | | | | |
| 16 | 100125_at | Cluster Incl U43918 Mus musculus proliferation-associated protein 1 mRNA, complete cds/cds = (266,1450)/gb = U43918/ | P | 448.1 | P | 379.9 | P | 436.1 | P |
| | | gi = 1167966/ug = Mm 4742/len = 1714/STRA = for | | | | | | | |
| 16 | 100594_at | Cluster Incl AB008895: Mus musculus mRNA for mGpI1p, complete cds/cds = (140,1885)/gb = AB008895/gi = 2641975/ | P | 237.4 | P | 211.7 | P | 257.6 | P |
| | | ug = Mm. 6354/len = 3062/STRA = for | | | | | | | |
| 16 | 100633_at | Cluster Incl AW124268: UI-M-BH2 1-aph-h-08-0-UI s1 Mus musculus cDNA, 3 end/clone = UI-M-BH2 1-aph-h-08-0-UI/ | P | 1022.5 | P | 834.3 | P | 984.2 | P |
| | | clone_end = 3/gb = AW124268/gi = 6099798/ug = Mm 6696/len = 385/STRA = for | | | | | | | |
| 16 | 101090_at | Cluster Incl L29454: Fibnllin 1/cds = (2379,10994)/gb = L29454/gi = 575509/ug = Mm.735/len = 11953/STRA = for | P | 1147.9 | P | 862.6 | P | 1756.5 | P |
| 16 | 101585_at | Cluster Incl AF042491 Mus musculus putative membrane associated progesterone receptor component mRNA, complete | P | 834 | P | 696.5 | P | 705.5 | P |
| | | cds/cds = (13,600)/gb = AF042491/gi = 2801792/ug = Mm 9052/len = 1786/STRA = for | | | | | | | |
| 16 | 101989_at | Cluster Incl AW125380 UI-M-BH2 3-aqh-b-06-0-UI.s1 Mus musculus cDNA, 3 end/clone = UI-M-BH2 3-aqh-b-06-0-UI/ | P | 2270.3 | P | 2186.2 | P | 2447.2 | P |
| | | clone_end = 3/gb = AW125380/gi = 6100910/ug = Mm 972/len = 269/STRA = for | | | | | | | |
| 16 | 102002_at | Cluster Incl AI836406 UI-M-AP0-abi-g-05-0-UI s1 Mus musculus cDNA, 3 end/clone = UI-M-AP0-abi-g-05-0-UI/ | P | 466.5 | P | 499.9 | P | 596.9 | P |
| | | clone_end = 3/gb = AI836406/gi = 5470619/ug = Mm 99026/len = 435/STRA = for | | | | | | | |
| 16 | 102061_at | Cluster Incl AF100171: Mus musculus myelodysplasia/myeloid leukemia factor 1 (Mlf1) mRNA, complete cds/cds = (60,863)/ | P | 198.6 | P | 176 | P | 253.1 | P |
| | | gb = AF100171/gi = 4106525/ug = Mm.10414/len = 879/STRA = for | | | | | | | |
| 16 | 102384_at | Cluster Incl AI842968 UI-M-AO1-aem-g-04-0-UI.s1 Mus musculus cDNA, 3 end/clone = UI-M-AO1-aem-g-04-0-UI/ | P | 249 | P | 202.1 | P | 252.7 | P |
| | | clone_end = 3/gb = AI842968/gi = 5477181/ug = Mm.12184/len = 467/STRA = for | | | | | | | |
| 16 | 102794_at | Cluster Incl Z80112 Mus musculus lcr-1 gene/cds = (0,1073)/gb = Z80112/gi = 2632100/ug = Mm 1401/len = 1180/STRA = for | P | 160.8 | P | 126.2 | P | 161.4 | P |
| 16 | 102859_at | Cluster Incl AW121304: UI-M-BH2.3-aog-g-02-0-UI s1 Mus musculus cDNA, 3 end/clone = UI-M-BH2 3-aog-g-02-0-UI/ | P | 418.5 | P | 258.6 | P | 400.3 | P |
| | | clone_end = 3/gb = AW121304/gi = 6096637/ug = Mm 14992/len = 261/STRA = for | | | | | | | |
| 16 | 103556_at | Cluster Incl AI840158 UI-M-AO0-acc-d-08-0-Ul.s1 Mus musculus cDNA, 3 end/clone = UI-M-AO0-aoc-d-08-0-UI/ | P | 795.4 | P | 883.6 | P | 1669.8 | P |
| | | clone_end = 3/gb = AI840158/gi = 5474371/ug = Mm 19081/len = 406/STRA = for | | | | | | | |
| 16 | 103570_at | Cluster Incl AI315647 uj31c11 x1 Mus musculus cDNA, 3 end/clone = IMAGE-1921556/clone_end = 3/gb = AI315647/ | P | 398.8 | P | 147.4 | P | 383.8 | P |
| | | gi = 4030914/ug = Mm 19310/len = 467/STRA = for | | | | | | | |
| 16 | 103592_at | Cluster Incl AB019374 Mus musculus mRNA for MEK5, complete cds/cds = (596,1942)/gb = AB019374/gi = 5360522/ | P | 290.5 | P | 243.1 | P | 368.1 | P |
| | | ug = Mm 19947/len = 2259/STRA = for | | | | | | | |
| 16 | 103650_at | Cluster Incl AI846887 UI-M-AK1-aey-a-01-0-UI s1 Mus musculus cDNA, 3 end/clone = UI-M-AK1-aey-a-01-0-UI/ | P | 328.1 | P | 325.6 | P | 402 | P |
| | | clone_end = 3/gb = AI846887/gi = 5490793/ug = Mm 20414/len = 320/STRA = for | | | | | | | |
| 16 | 103812_at | Cluster Incl AF047838: Mus musculus calcium-sensitive chlonde conductance protein-1 (mCLCA1) mRNA, complete cds/ | P | 319.6 | P | 238.9 | P | 360.3 | P |
| | | cds = (17,2725)/gb = AF047838/gi = 3925280/ug = Mm 20897/len = 3022/STRA = rev | | | | | | | |
| 16 | 103829_at | Cluster Incl AF061529 Mus musculus rjs (rjs) mRNA, complete cds/cds = (119,14629)/gb = AF061529/gi = 3414808/ | P | 107.4 | P | 70 | P | 83.3 | P |
| | | ug = Mm 20929/len = 15264/STRA = for | | | | | | | |
| 16 | 104345_at | Cluster Incl AF014461 Mus musculus EXO70 protein (Exo70) mRNA, complete cds/cds = (0,2093)/gb = AF014461/ | P | 268.7 | P | 252.8 | P | 374.9 | P |
| | | gi = 2352997/ug = Mm 22530/len = 2094/STRA = for | | | | | | | |
| 16 | 104365_at | Cluster Incl AW046162 UI-M-BH1-akw-b-04-0-UI s1 Mus musculus cDNA, 3 end/clone = UI-M-BH1-akw-b-04-0-UI/ | P | 475.8 | P | 367.4 | P | 465.4 | P |
| | | clone_end = 3/gb = AW046162/gi = 5906691/ug = Mm 22615/len = 456/STRA = for | | | | | | | |
| 16 | 104572_at | Cluster Incl AI852047: UI-M-BH0-aja-b-01-0-UI s1 Mus musculus cDNA, 3 end/clone = UI-M-BH0-aja-b-01-0-UI/ | P | 493.6 | P | 353.2 | P | 548.2 | P |
| | | clone_end = 3/gb = AI852047/gi = 5495953/ug = Mm 23910/len = 523/STRA = for | | | | | | | |
| 16 | 104725_at | Cluster Incl AW060401 UI-M-BH1-anj-a-06-0-UI s1 Mus musculus cDNA, 3 end/clone = UI-M-BH1-anj-a-06-0-UI/ | P | 558 | P | 378.1 | P | 543.7 | P |
| | | clone_end = 3/gb = AW060401/gi = 6008152/ug = Mm 24574/len = 467/STRA = for | | | | | | | |
| 16 | 104746_at | Cluster IncI AF040252 Mus musculus FK506-binding protein (FKBP23) mRNA, complete cds/cds = (56,712)/gb = AF040252/ | P | 354.7 | P | 207.4 | P | 347.3 | P |
| | | gi = 2827254/ug = Mm 24720/len = 866/STRA = for | | | | | | | |
| 16 | 94331_at | Cluster Incl L47650 Signal transducer and activator of transcription 6/cds = (279,2792)/gb = L47650/gi = 1008876/ | P | 367.9 | P | 187.7 | P | 502.9 | P |
| | | ug = Mm 2489/len = 3213/STRA = for | | | | | | | |
| 16 | 94348_f_at | Cluster Incl AW124044 UI-M-BH2.1-apn-d-10-0-UI s1 Mus musculus cDNA, 3 end/clone = UI-M-BH2 1-apn-d-10-0-UI/ | P | 499.8 | P | 495.1 | P | 612.8 | P |
| | | clone_end = 3/gb = AW124044/gi = 6099574/ug = Mm.25169/len = 408/STRA = for | | | | | | | |
| 16 | 94392_f_at | Cluster Incl U22516: Angiogenin/cds = (0,437)/gb = U22516/gi = 726325/ug = Mm 2566/len = 438/STRA = for | P | 163.6 | P | 114.1 | P | 295.7 | P |
| 16 | 94422_at | Cluster Incl AI842938 UI-M-AO1-aem-d-04-0-UI.s1 Mus musculus cDNA, 3 end/clone = UI-M-AO1-aem-d-04-0-UI/ | P | 203 | P | 212.4 | P | 207.5 | P |
| | | clone_end = 3/gb = AI842938/gi = 5477151/ug = Mm.26242/len = 303/STRA = for | | | | | | | |
| 16 | 94930_at | Cluster Incl L07803: Thrombospondin 2/cds = (218,3736)/gb = L07803/gi = 340421/ug = Mm 26688/len = 4108/STRA = for | P | 716 | P | 254.4 | P | 655.4 | P |
| 16 | 94976_at | Cluster Incl AI845877 Ul-M-AO1-aeg-g-12-0-UI s1 Mus musculus cDNA, 3 end/clone = UI-M-AO1-aog-g-12-0-UI/ | P | 401.1 | P | 340.9 | P | 705.5 | P |
| | | clone_end = 3/gb = AI845877/gi = 5489783/ug = Mm.27259/len = 409/STRA = rev | | | | | | | |
| 16 | 94995_at | Cluster Incl AI854331 UI-M-BG1-aic-b-07-0-UI s1 Mus musculus cDNA, 3 end/clone = UI-M-BG1-aic-b-07-0-UI/ | P | 1472.7 | P | 654.7 | P | 1548.9 | P |
| | | clone_end = 3/gb = AI854331/gi = 5498237/ug = Mm.27337/len = 404/STRA = rev | | | | | | | |
| 16 | 95014_at | Cluster Incl AA794247: vu65b08 r1 Mus musculus cDNA, 5 end/clone = IMAGE-1196247/clone_end = 5/gb = AA794247/ | P | 536 | P | 456.6 | P | 1130.5 | P |
| | | gi = 2857202/ug = Mm.27445/len = 496/STRA = for | | | | | | | |
| 16 | 95529_at | Cluster Incl U58884 Mus musculus SH3-containing protein SH3P7 mRNA, complete cds similar to Human Drebrin/ | P | 746 | P | 625.3 | P | 923.9 | P |
| | | cds = (30,1331)/gb = U58884/gi = 1407654/ug = Mm 27622/len = 2291/STRA = rev | | | | | | | |
| 16 | 95565_at | Cluster Incl AI852873 UI-M-BH0-ajk-f-03-0-Ul.s1 Mus musculus cDNA, 3 end/clone = UI-M-BH0-ajk-f-03-0-UI/clone_end = 3/ | P | 547.7 | P | 533.7 | P | 600.6 | P |
| | | gb = AI852873/gi = 5496779/ug = Mm 27787/len = 327/STRA = for | | | | | | | |
| 16 | 95590_at | Cluster Incl AA615951: vo95c11.r1 Mus musculus cDNA, 5 end/clone = IMAGE-1066868/clone_end = 5/gb = AA615951/ | P | 223.5 | P | 199.7 | P | 258 | P |
| | | gi = 2503187/ug = Mm 27890/len = 621/STRA = for | | | | | | | |
| 16 | 95597_at | Cluster Incl M34141 Prostaglandin-endoperoxide synthase 1/cds = (36,1844)/gb = M34141/gi = 200302/ug = Mm.2792/ | P | 227.4 | P | 178.1 | P | 304.4 | P |
| | | len = 2757/STRA = for | | | | | | | |
| 16 | 95617_at | Cluster Incl U36799 Retinoblastoma-like 2/cds = (19,3396)/gb = U36799/gi = 1045519/ug = Mm 28027/len = 4806/STRA = for | P | 124 | P | 57.2 | P | 120.2 | P |
| 16 | 95622_at | Cluster Incl AW123907 UI-M-BH2 1-app-h-01-0-UI.s1 Mus musculus cDNA, 3 end/clone = UI-M-BH2.1-app-h-01-0-UI/ | P | 396.2 | P | 375.7 | P | 447.8 | P |
| | | clone_end = 3/gb = AW123907/gi = 6099437/ug = Mm 28047/len = 326/STRA = for | | | | | | | |
| 16 | 95630_at | Cluster Incl AW122852 UI-M-BH2 1-aoz-g-07-0-UI s1 Mus musculus cDNA, 3 end/clone = UI-M-BH2 1-aoz-g-07-0-UI/ | P | 478.7 | P | 424.9 | P | 718.4 | P |
| | | clone_end = 3/gb = AW122852/gi = 6098382/ug = Mm 28080/len = 351/STRA = rev | | | | | | | |
| 16 | 96127_at | Cluster Incl AW048730: UI-M-BH1-amy-e-02-0-UI s1 Mus musculus cDNA, 3 end/clone = UI-M-BH1-amy-e-02-0-UI/ | P | 854.9 | P | 762.5 | P | 1267.1 | P |
| | | clone_end = 3/gb = AW048730/gi = 5909259/ug = Mm 28119/len = 536/STRA = for | | | | | | | |
| 16 | 96765_at | Cluster Incl AW120874: UI-M-BH2.3-aoa-h-02-0-UI.s1 Mus musculus cDNA, 3 end/clone = UI-M-BH2.3-aoa-h-02-0-UI/ | P | 1916.5 | P | 1794.6 | P | 2201.1 | P |
| | | clone_end = 3/gb = AW120874/gi = 6096207/ug = Mm.29009/len = 298/STRA = for | | | | | | | |
| 16 | 96802_at | Cluster Incl AW124656: UI-M-BH2 1-apw-e-04-0-U1.s1 Mus musculus cDNA, 3 end/clone = UI-M-BH2.1-apw-e-04-0-UI/ | P | 150 | P | 87.2 | P | 148.6 | P |
| | | clone_end = 3/gb = AW124656/gi = 6100186/ug = Mm.29193/len = 368/STRA = for | | | | | | | |
| 16 | 97977_at | Cluster Incl AA645293 vs81a07.r1 Mus musculus cDNA, 5 end/clone = IMAGE-1152660/clone_end = 5/gb = AA645293/ | P | 487.4 | P | 376.5 | P | 581.5 | P |
| | | gi = 2571722/ug = Mm 3116/len = 276/STRA = for | | | | | | | |
| 16 | 98468_r_at | Cluster Incl AI316859: ul35b03 y1 Mus musculus cDNA, 5 end/clone = IMAGE-1853261/clone_end = 5/gb = AI316659/ | P | 325.1 | P | 208.7 | P | 329.7 | P |
| | | gi = 4032126/ug = Mm 34879/len = 500/STRA = for | | | | | | | |
| 16 | 98511_at | Cluster Incl L17076 HnRNP-associated with lethal yellow/cds = UNKNOWN/gb = L17076/gi = 348995/ug = Mm 3591/ | P | 433.5 | P | 415.4 | P | 644.6 | P |
| | | len = 1517/STRA = for | | | | | | | |
| 16 | 99010_at | Cluster Incl AB024538 Mus musculus Islr(immunoglobulin superfamily containing leucine-nch repeat) mRNA, complete cds/ | P | 426.3 | P | 372.8 | P | 1018.3 | P |
| | | cds = (89,1375)/gb = AB024538/gi = 6172374/ug = Mm 38426/len = 2038/STRA = for | | | | | | | |
| 16 | 99029_at | Cluster Incl AF077950: Mus musculus protein inhibitor of activated STAT protein PIAS1 mRNA, complete cds/cds = (0,1955)/ | P | 226.1 | P | 311.3 | P | 430.6 | P |
| | | gb = AF077950/gi = 3643104/ug = Mm 38869/len = 1956/STRA = for | | | | | | | |
| 16 | 99067_at | Cluster Incl X59846 M. musculus GAS 6 mRNA associated with growth-arrest/cds = (167,2188)/gb = X59846/gi = 407060/ | P | 331.5 | P | 264.2 | P | 544 | P |
| | | ug = Mm 3982/len = 2573/STRA = rev | | | | | | | |
| 16 | 99476_at | Cluster Incl AJ131395: Mus musculus mRNA for collagen type XIV, partial/cds = (0,380)/gb = AJ131395/gi = 5042293/ | P | 520.5 | P | 519.3 | P | 956.2 | P |
| | | ug = Mm.41332/len = 1171/STRA = for | | | | | | | |
| 16 | 99518_at | Cluster Incl AW121179 UI-M-BH2 3-aok-a-06-0-UI s1 Mus musculus cDNA, 3 end/clone = UI-M-BH2 3-aok-a-06-0-UI/ | P | 678.3 | P | 723.7 | P | 1719.7 | P |
| | | clone_end = 3/gb = AW121179/gi = 6096512/ug = Mm 41931/len = 479/STRA = for | | | | | | | |
| 16 | 99940_at | Cluster Incl AW212667 uo10c01 x1 Mus musculus cDNA, 3 end/clone = IMAGE-2599104/clone_end = 3/gb = AW212667/ | P | 286.9 | P | 236.7 | P | 318.6 | P |
| | | gi = 6518780/ug = Mm 435/len = 464/STRA = for | | | | | | | |
| 16 | 99947_at | Cluster Incl U55861 Mus musculus RNA binding protein TIAR mRNA, complete cds/cds = (133,1311)/gb = U55861/ | P | 464.1 | P | 366.8 | P | 437.3 | P |
| | | gi = 1592562/ug = Mm 4363/len = 1543/STRA = for | | | | | | | |
| 16 | 100443_at | Cluster Incl AF031467 Branched chain aminotransferase 2, mitochondrial/cds = (135,1196)/gb = AF031467/gi = 3298578/ | P | 297.3 | P | 219.5 | P | 243.9 | P |
| | | ug = Mm 4811/len = 1200/STRA = for | | | | | | | |
| 16 | 100477_at | Cluster Incl M32486 Mouse 19.5 mRNA, complete cds/cds = (54,875)/gb = M32486/gi = 191495/ug = Mm 4992/len = 1183/ | P | 1808.9 | P | 1689.7 | P | 2073.6 | P |
| | | STRA = for | | | | | | | |
| 16 | 101416_f_at | Cluster Incl L76155 Mus musculus Bat-4 gene, complete cds/cds = (40,1158)/gb = L76155/gi = 1196643/ug = Mm 7405/ | P | 694.6 | P | 529.9 | P | 700.4 | P |
| | | len = 1400/STRA = for | | | | | | | |
| 16 | 104408_s_at | Cluster Incl L35032 SRY-box containing gene 18/cds = (88,1221)/gb = L35032/gi = 1663531/ug = Mm 2878/len = 1585/ | P | 478 | P | 285.4 | P | 446.7 | P |
| | | STRA = for | | | | | | | |
| 16 | 104445_at | Cluster Incl AW046694 UI-M-BH1-alm-d-07-0-UI s1 Mus musculus cDNA, 3 end/clone = UI-M-BH1-alm-d-07-0-UI/ | P | 1312.3 | P | 916.4 | P | 1489 | P |
| | | clone_end = 3/gb = AW046694/gi = 5907223/ug = Mm 2935/len = 253/STRA = for | | | | | | | |
| 16 | 92207_at | Cluster Incl U08210 Elastin/cds = (0,2582)/gb = U08210/gi = 473273/ug = Mm 34170/len = 2651/STRA = for | P | 647.1 | P | 622.1 | P | 919 | P |
| 16 | 92224_at | Cluster Incl X79199 Tetranectin (plasminogen-binding protein)/cds = (73,681)/gb = X79199/gi = 671501/ug = Mm 34588/ | P | 373.7 | P | 636.5 | P | 973.3 | P |
| | | len = 988/STRA = rev | | | | | | | |
| 16 | 92375_at | Cluster Incl AW211207 ut29a03 y1 Mus musculus cDNA, 5 end/clone = IMAGE-2099692/clone_end = 5/gb = AW211207/ | P | 284.7 | P | 318.3 | P | 386.7 | P |
| | | gi = 6517147/ug = Mm 4002/len = 531/STRA = for | | | | | | | |
| 16 | 93443_at | Cluster Incl AW212271 uo84g08 x1 Mus musculus cDNA, 3 end/clone = IMAGE-2649278/clone_end = 3/gb = AW212271/ | P | 298.2 | P | 401.2 | P | 572.8 | P |
| | | gi = 6515306/ug = Mm 6671/len = 420/STRA = for | | | | | | | |
| 16 | 93472_at | Cluster Incl AA796989: vt01a04 r1 Mus musculus cDNA, 5 end/clone = IMAGE-1154478/clone_end = 5/gb = AA796989/ | P | 1697.6 | P | 1395.2 | P | 2233.5 | P |
| | | gi = 2859944/ug = Mm.7049/len = 428/STRA = for | | | | | | | |
| 16 | 93485_at | Cluster Incl AI844911 UI-M-BG0-ahv-d-10-0-UI s1 Mus musculus cDNA, 3 end/clone = UI-M-BG0-ahv-d-10-0-UI/ | P | 205.5 | P | 166.4 | P | 157.5 | P |
| | | clone_end = 3/gb = AI844911/gi = 5488617/ug = Mm 7172/len = 399/STRA = for | | | | | | | |
| 16 | 93615_at | Cluster Incl AF020200 Mus musculus transcription factor PBX3b (PBX3b) mRNA, complete cds/cds = (118,1173)/ | P | 201.1 | P | 181.2 | P | 167.1 | P |
| | | gb = AF020200/gi = 2432016/ug = Mm.7331/len = 2467/STRA = for | | | | | | | |
| 16 | 93874_s_at | Cluster Incl U69491: lnterleukin 11 receptor, alpha chain 2/cds = (301,1599)/gb = U69491/gi = 1916003/ug = Mm 88405/ | P | 773.8 | P | 534.9 | P | 769.4 | P |
| | | len = 1975/STRA = for | | | | | | | |
| 16 | 99812_at | Cluster Incl X92523 Calpain 3/cds = (0,2465)/gb = X92523/gi = 1246778/ug = Mm 57235/len = 2466/STRA = for | P | 211.7 | P | 201.6 | P | 287.4 | P |
| 16 | 94642_at | Cluster Incl U07951 Mus musculus BALB/c GDP-dissociation inhibitor (GDI-2) mRNA, complete cds/cds = (289,1626)/ | P | 135 | P | 111.6 | P | 283.6 | P |
| | | gb = U07951/gi = 1486479/ug = Mm 8070/len = 1676/STRA = for | | | | | | | |
| 16 | 93835_at | Cluster Incl AI843222: UI-M-AK1-aet-g-05-0-UI s1 Mus musculus cDNA, 3 end/clone = UI-M-AK1-ael-g-05-0-UI/ | P | 267.1 | P | 175.9 | P | 282.4 | P |
| | | clone_end = 3/gb = AI843222/gi = 5477435/ug = Mm.21580/len = 373/STRA = for | | | | | | | |
| 16 | 94079_at | Cluster Incl X61452 M musculus mRNA for H5 clone/cds = (35,1471)/gb = X61452/gi = 51202/ug = Mm 2214/len = 1649/ | P | 158.7 | P | 105.3 | A | 303.3 | P |
| | | STRA = for | | | | | | | |
| 16 | 94275_at | Cluster Incl U34691 Uroporphynnogen decarboxylase/cds = (281,1384)/gb = U34691/gi = 1498642/ug = Mm 22494/len = 1454/ | P | 198.9 | P | 186.1 | P | 215.7 | P |
| | | STRA = for | | | | | | | |
| 16 | 94304_at | Cluster Incl X13460: Annexin VI, p68/cds = (33,2054)/gb = X13460/gi = 53580/ug = Mm 22619/len = 2402/STRA = for | P | 736 | P | 804.7 | P | 838.2 | P |
| 16 | 97835_at | Cluster Incl AI842828 UI-M-AP1-agi-h-10-0-UI s1 Mus musculus cDNA, 3 end/clone = UI-M-Ap1-agi-h-10-0-UI/ | P | 684.8 | P | 633.6 | P | 806.5 | P |
| | | clone_end = 3/gb = AI842828/gi = 5477041/ug = Mm28231/len = 175/STRA = for | | | | | | | |
| 16 | 98923_at | Cluster Incl AI852608 UI-M-BH0-alu-d-07-0-UI s1 Mus musculus cDNA, 3 end/clone = UI-M-BH0-alu-d-07-0-UI/ | P | 113.6 | P | 44.6 | P | 235.3 | P |
| | | clone_end = 3/gb = AI852608/gi = 5496514/ug = Mm 28630/len = 273/STRA = for | | | | | | | |
| 16 | 94459_at | Cluster Incl AA681764 v161b04 r1 Mus musculus cDNA, 5 end/clone = IMAGE-1167535/clone_end = 5/gb = AA681764/ | P | 143.7 | P | 144 | P | 179.5 | P |
| | | gi = 2663904/ug = Mm.28818/len = 441/STRA = for | | | | | | | |
| 16 | 96296_at | Cluster Incl AI843685 UI-M-AK1-aer-c-07-0-UI s1 Mus musculus cDNA, 3 end/clone = UI-M-AK1-aer-c-07-0-UI/ | P | 367.9 | P | 409.1 | P | 486.1 | P |
| | | clone_end = 3/gb = AI843685/gi = 5477898/ug = Mm.29904/len = 305/STRA = for | | | | | | | |
| 16 | 99630_at | Cluster Incl AW060257 UI-M-BH1-amv-f-08-0-UI.s1 Mus musculus cDNA, 3 end/clone = UI-M-BH1-amv-f-08-0-UI/ | P | 156.2 | P | 195.1 | P | 264.9 | P |
| | | clone_end = 3/gb = AW060257/gi = 6008048/ug = Mm.43397/1en = 434/STRA = for | | | | | | | |
| 16 | 101036_at | Cluster Incl AW047047 UI-M-BH1-alr-c-02-0-UI s1 Mus musculus cDNA, 3 end/clone = UI-M-BH1-alr-c-02-0-UI/ | P | 436.9 | P | 414. 6 | P | 625.4 | P |
| | | clone_end = 3/gb = AW047047/gi = 5907576/ug = Mm.6932/len = 334/STRA = for | | | | | | | |
| 16 | 103030_at | Cluster Incl L31397: Dynamin/cds = (73,2658)/gb = L31397/gi = 487856/ug = Mm 16080/len = 3924/STRA = rev | M | 367 | P | 122 | P | 531.9 | P |
| 16 | 104610_at | Cluster Incl AW050015: UI-M-BH1-amm-a-01-0-UI.s1 Mus musculus cDNA, 3 end/clone = UI-M-BH1-amm-a-01-0-UI/ | P | 156.4 | P | 132.1 | A | 184.5 | P |
| | | clone_end = 3/gb = AW050015/gi = 5910544/ug = Mm.2415/len = 503/STRA = rev | | | | | | | |
| 16 | 104616_g_at | Cluster Incl M96265: Mus musculus galactose-1-phosphate uridyl transferase (GALT) mRNA, complete cds/cds = (131,1312)/ | P | 124.7 | P | 29.3 | P | 200.1 | P |
| | | gb = M96265/gi = 193421/ug = Mm 2420/len = 1373/STRA = for | | | | | | | |
| 16 | 94428_at | Cluster Incl AI842668 UI-M-AO1-aeo-a-05-0-UI s1 Mus musculus cDNA, 3 end/clone = UI-M-AO1-aeo-a-05-0-UI/ | P | 268.6 | P | 151.4 | A | 412.6 | P |
| | | clone_end = 3/gb = AI842668/gi = 5476881/ug = Mm 2644/len = 378/STRA = for | | | | | | | |
| 16 | 95537_at | Cluster Incl AB019577 Mus musculus mRNA for UNC-51-like kinase (ULK) 2, complete cds/cds = (41,3154)/gb = AB019577/ | P | 63.2 | P | 83.3 | P | 180 | P |
| | | gi = 4760560/ug = Mm 27664/len = 5275/STRA = for | | | | | | | |
| 16 | 96728_at | Cluster Incl AI844151 UI-M-AL1-ahj-f-07-0-UI.s1 Mus musculus cDNA, 3 end/clone = UI-M-AL1-ahj-f-07-0-UI/clone_end = 3/ | P | 265.8 | P | 212.5 | P | 363.8 | P |
| | | gb = AI844151/gi = 5488057/ug = Mm 28850/len = 317/STRA = rev | | | | | | | |
| 16 | 99929_at | Cluster Incl U72141: Exostoses (multiple) 2/cds = (191,2347)/gb = U72141/gi = 1619953/ug = Mm 4338/len = 2864/STRA = for | P | 379.4 | P | 327 | A | 476.9 | P |
| 16 | 99992_at | Cluster Incl AI286698 ub84b09 r1 Mus musculus cDNA, 5 end/clone = IMAGE-1395161/clone_end = 5/gb = AI286698/ | P | 277.2 | P | 297.9 | A | 388.3 | P |
| | | gi = 3926451/ug = Mm.4481/len = 200/STRA = for | | | | | | | |
| 16 | 100468_g_at | Cluster Incl X57667 M musculus LYL gene (clone L6)/cds = (465,1301)/gb = X57687/gi = 52965/ug = Mm 4925/len = 1686/ | P | 161.9 | P | 64.7 | M | 150.1 | P |
| | | STRA = for | | | | | | | |
| 16 | 101446_at | Cluster Incl AF004428, Turnor protein D52-like 1/cds = (191,805)/gb = AF004428/gi = 2895080/ug = Mm.7821/len = 1267/ | P | 128 | P | 116.5 | P | 151.1 | A |
| | | STRA = for | | | | | | | |
| 16 | 102720_at | Cluster Incl X71426 Endothelial-specific receptor tyrosine kinase/cds = (86 3454)/gb = X71426/gi = 296612/ug = Mm 14313/ | P | 165.4 | P | 111.8 | A | 201.9 | P |
| | | len = 3490/STRA = for | | | | | | | |
| 16 | 103536_at | Cluster Incl AB017270 Mus musculus TMEFF2 mRNA, complete cds/cds = (388,1512)/gb = AB017270/gi = 6518464/ | P | 146.9 | P | 54.6 | P | 108 | P |
| | | ug = Mm 23304/len = 1740/STRA = for | | | | | | | |
| 16 | 103922_f_at | Cluster Incl AI839690 UI-M-AN0-acp-e-04-0-UI s1 Mus musculus cDNA, 3 end/clone = UI-M-AN0-acp-e-04-0-UI/ | P | 202.7 | P | 180 | P | 198.2 | P |
| | | clone_end = 3/gb = AI839690/gi = 5473903/ug = Mm 25034/len = 250/STRA = rev | | | | | | | |
| 16 | 104680_at | Cluster Incl AJ250489 Mus musculus mRNA for receptor activity modifying protein 1 (Ramp1 gene)/cds = (59,505)/ | P | 408.2 | P | 325.2 | P | 669.8 | P |
| | | gb = AJ250489/gi = 6119624/ug = Mm 3272/len = 844/STRA = for | | | | | | | |
| 16 | 92366_at | Cluster Incl U12147.Laminin, alpha 2/cds = (54,9374)/gb = U12147/gi = 699109/ug = Mm 3987/len = 9511/STRA = for | P | 586.2 | P | 370.2 | P | 518.1 | P |
| 16 | 92397_at | Cluster Incl AW123016 UI-M-BH2 1-apb-e-06-0-UI s1 Mus musculus cDNA, 3 end/clone = UI-M-BH2.1-apb-e-06-0-UI/ | P | 571.9 | P | 424.6 | P | 677.8 | P |
| | | clone_end = 3/gb = AW123016/gi = 6098546/ug = Mm 4077/len = 473/STRA = rev | | | | | | | |
| 16 | 92739_at | Cluster Incl L28819 Involucnn/cds = (0,1403)/gb = L28819/gi = 454418/ug = Mm.46792/len = 1404/STRA = for | P | 370.7 | P | 127.4 | P | 322.7 | P |
| 16 | 92894_s_at | Cluster Incl D90174 Mouse mRNA for NFI-B protein, clone NFI-B3/cds = (237,1742)/gb = D90174/gi = 220520/ug = Mm 4771/ | P | 169.9 | P | 119.7 | A | 242.2 | P |
| | | len = 1859/STRA = for | | | | | | | |
| 16 | 93421_at | Cluster Incl AF033655 PFTAIRE protein kinase 1/cds = (85,1494)/gb = AF033655/gi = 2645809/ug = Mm 6456/len = 4529/ | P | 128.4 | P | 97.5 | P | 198.7 | P |
| | | STRA = for | | | | | | | |
| 16 | 97155_at | Cluster Incl U84005 Myostatin/cds = (103,1233)/gb = U84005/gi = 2065554/ug = Mm.3514/len = 2676/STRA = for | P | 275.4 | P | 230.8 | P | 509.5 | P |
| 16 | 97874_at | Cluster Incl AI853863 UI-M-BH0-ajr-f-07-0-UI.s1 Mus musculus cDNA, 3 end/clone = UI-M-BH0-ajr-f-07-0-UI/clone_end = 3/ | P | 307.3 | P | 372.7 | P | 417.3 | A |
| | | gb = AI853863/gi = 5497769/ug = Mm 28349/len = 440/STRA = for | | | | | | | |
| 16 | 95693_at | Cluster Incl U51167 Isocitrate dehydrogenase 2/cds = (23,1594)/gb = U51167/gi = 1236983/ug = Mm 2966/len = 1863/ | P | 690.3 | P | 485 | A | 1027.4 | P |
| | | STRA = for | | | | | | | |
| 16 | 98144_f_at | Cluster Incl AI844321 UI-M-AL1-ahm-f-07-0-UI s1 Mus musculus cDNA, 3 end/clone = UI-M-AL1-ahm-f-07-0-UI/ | P | 308.3 | P | 192.2 | P | 564 | P |
| | | clone_end = 3/gb = AI844321/gi = 5488227/ug = Mm 35650/len = 381/STRA = for | | | | | | | |
| 16 | 102029_at | Cluster Incl AF017111 Interleukin 16/cds = (168,2042)/gb = AF017111/gi = 3127042/ug = Mm 10137/len = 2876/STRA = rev | P | 129.1 | P | 88.3 | P | 177.8 | P |
| 16 | 103637_at | Cluster Incl AJ223966 N-acetyl galactosaminidase, alpha/cds = (0,1247)/gb = AJ223966/gi = 3327015/ug = Mm 20325/ | P | 234.7 | P | 190.2 | M | 281.9 | P |
| | | len = 1248/STRA = for | | | | | | | |
| 16 | 94417_at | Cluster Incl AI852949 UI-M-BH0-aiz-e-01-0-UI s1 Mus musculus cDNA, 3 end/clone = UI-M-BH0-aiz-e-01-0-UI/ | P | 95.1 | P | 67.6 | P | 124.5 | A |
| | | clone_end = 3/gb = AI852949/gi = 5496855/ug = Mm 26168/len = 338/STRA = for | | | | | | | |
| 16 | 96176_at | Cluster Incl AI847916 UI-M-AI1-afl-c-02-0-UI s1 Mus musculus cDNA, 3 end/clone = UI-M-AI1-afl-c-02-0-UI/clone_end = 3/ | P | 341.8 | P | 102.7 | P | 536 | P |
| | | gb = AI847916/gi = 5491822/ug = Mm 28409/len = 447/STRA = for | | | | | | | |
| 16 | 98449_at | Cluster Incl AB014721 Mus musculus mRNA for mSART-1(806), complete cds/cds = (34,2454)/gb = AB014721/gi = 4126468/ | P | 111.4 | P | 58.3 | A | 117 | A |
| | | ug = Mm 34562/len = 2513/STRA = rev | | | | | | | |
| 16 | 100943_at | Cluster Incl U75215 Mus musculus neutral amino acid transporter mASCT1 mRNA, complete cds/cds = = (111,1709)/ | P | 222.5 | P | 302.4 | P | 278 | P |
| | | gb = U75215/gi = 2459560/ug = Mm 6379/len = 3787/STRA = for | | | | | | | |
| 16 | 102227_g_at | Cluster Incl AF017639 Mus musculus carboxypeptidase X2 mRNA, complete cds/cds = (129,2423)/gb = AF017639/ | A | 111.6 | P | 78.9 | A | 120.3 | P |
| | | gi = 2921091/ug = Mm 10233/len = 2490/STRA = for | | | | | | | |
| 16 | 103502_at | Cluster Incl AA866768 ud07g06 r1 Mus musculus cDNA, 5 end/clone = IMAGE-1434490/clone_end = 5/gb = AA866768/ | A | 147.8 | P | 152.9 | P | 152.7 | P |
| | | gi = 2962213/ug = Mm 22367/len = 376/STRA = for | | | | | | | |
| 16 | 92450_at | Cluster Incl AF047339: Solute camer family 12, member 4/cds = (0,3257)/gb = AF047339/gi = 3452286/ug = Mm 4190/ | A | 283.7 | P | 394.2 | P | 500.2 | P |
| | | len = 3775/STRA = for | | | | | | | |
| 16 | 97784_at | Cluster Incl Y14695 Mus musculus mRNA for epidermis-type lipoxygenase-3/cds = (26,2161)/gb = Y14695/gi = 5304927/ | P | 340.8 | P | 144.9 | P | 762.5 | P |
| | | ug = Mm 41989/len = 2536/STRA = for | | | | | | | |
| 16 | 100743_at | Cluster Incl AA407663 EST01744 Mus musculus cDNA, 3 end/clone = C0013E09/clone_end = 3/gb = AA407663/ | A | 146.4 | P | 103.2 | A | 146.4 | P |
| | | gi = 2066659/ug = Mm 3640/len = 265/STRA = rev | | | | | | | |
| 16 | 94504_at | Cluster Incl AI838655 UI-M-AO0-aca-c-08-0-UI.s1 Mus musculus cDNA, 3 end/clone = UI-M-AO0-aca-c-08-0-UI/ | P | 171.5 | P | 313.2 | M | 448.9 | A |
| | | clone_end = 3/gb = AI838655/gi = 5472868/ug = Mm 28955/len = 343/STRA = rev | | | | | | | |
| 16 | 95642_at | Cluster Incl AI842813 UI-M-AP1-agi-g-02-0-UI.s1 Mus musculus cDNA, 3 end/clone = UI-M-AP1-agi-g-02-0-UI/ | P | 200.7 | A | 251.1 | A | 851.1 | P |
| | | clone_end = 3/gb = AI842813/gi = 5477026/ug = Mm 29492/len = 403/STRA = for | | | | | | | |
| 16 | 99156_at | Cluster Incl AI853370 UI-M-BH0-air-e-04-0-UI s1 Mus musculus cDNA, 3 end/clone = UI-M-BH0-air-e-04-0-UI/ | P | 115.5 | P | 100.7 | P | 165.1 | A |
| | | clone_end = 3/gb = AI853370/gi = 5497276/ug = Mm.41617/len = 415/STRA = for | | | | | | | |
| 16 | 101571_g_at | Cluster Incl X76066 M musculus gene for insulin like growth factor binding protein 4/cds = (0,764)/gb = X76066/gi = 416032/ | P | 784.4 | P | 671.7 | P | 937.6 | A |
| | | ug = Mm 89842/len = 765/STRA = for | | | | | | | |
| 16 | 102343_at | Cluster Incl AF110520: Mus musculus major histocompatibility complex region NG27, NG28, RPS28, NADH | P | 374.9 | P | 285.4 | P | 539.7 | A |
| | | oxidoreductase, NG29, KIFC1, Fas-binding protein, BING1, tapasin, RaIGDS-like, KE2, | | | | | | | |
| | | BING4, beta 1,3-galactosyl transferase, and RPS18 genes, complete cds; S/STRA = for | | | | | | | |
| 16 | 103017_at | Cluster Incl AI060729 ub42h10 r1 Mus musculus cDNA, 5 end/clone = IMAGE-1380451/clone_end = 5/gb = AI060729/ | P | 142.5 | P | 75.1 | A | 163 | M |
| | | gi = 3336152/ug = Mm 1565/len = 457/STRA = for | | | | | | | |
| 16 | 97964_at | Cluster Incl AW122851: UI-M-BH2 1-aoz-g-06-0-UI s1 Mus musculus cDNA, 3 end/clone = UI-M-BH2 1-aoz-g-06-0-UI/ | P | 146.3 | P | 20 | A | 78.2 | A |
| | | clone_end = 3/gb = AW122851/gi = 6098381/ug = Mm 30729/len = 462/STRA = for | | | | | | | |
| 16 | 100408_at | Cluster Incl AA839465-vw50d06 r1 Mus musculus cDNA, 5 end/clone = IMAGE-1247243/clone_end = 5/gb = AA839465/ | P | 117 | P | 102.5 | A | 188.6 | A |
| | | gi = 2915560/ug = Mm 46563/len = 437/STRA = for | | | | | | | |
| 16 | 100991_at | Cluster Incl AI852849 UI-M-BH0-ajk-d-01-0-UI s1 Mus musculus cDNA, 3 end/clone = UI-M-BH0-ajk-d-01-0-UI/ | M | 225.4 | P | 211.8 | P | 324 | A |
| | | clone_end = 3/gb = AI852849/gi = 5496755/ug = Mm 6818/len = 348/STRA = for | | | | | | | |
| 16 | 101901_at | Cluster Incl AB024713 Mus musculus mRNA for Supl15h, complete cds/cds = (6,749)/gb = AB024713/gi = 5103141/ | P | 317.6 | P | 182.1 | P | 334.4 | P |
| | | ug = Mm 89579/len = 1249/STRA = for | | | | | | | |
| 16 | 102969_at | Cluster Incl AI847369 UI-M-AN1-afh-g-04-0-UI s1 Mus musculus cDNA, 3 end/clone = UI-M-AN1-afh-g-04-0-UI/ | P | 126.1 | A | 101.2 | P | 168.4 | P |
| | | clone_end = 3/gb = AI847369/gi = 5491240/ug = Mm 183/len = 436/STRA = for | | | | | | | |
| 16 | 103273_s_at | Cluster Incl AF037312 untitled/cds = (0,2051)/gb = AF037312/gi = 3851489/ug = Mm 20868/len = 2147/STRA = for | P | 114 | P | 119.6 | P | 221.6 | P |
| 16 | 96577_i_at | Cluster Incl AI853019 UI-M-BH0-ajn-d-04-0-UI s1 Mus musculus cDNA, 3 end/clone = UI-M-BH0-ajn-d-04-0-UI/ | P | 145.7 | P | 100.2 | M | 243.7 | P |
| | | clone_end = 3/gb = AI853019/gi = 5496925/ug = Mm 3021/len = 250/STRA = for | | | | | | | |
| 16 | 98859_at | Cluster Incl M99054 Acid phosphatase 5, tartrate resistant/cds = (132,1115)/gb = M99054/gi = 553868/ug = Mm.5119/ | P | 237.8 | P | 182.3 | P | 266.1 | A |
| | | len = 1386/STRA = for | | | | | | | |
| 16 | 101703_at | Cluster Incl V00716 Mouse pseudogene for alpha-globin without intervening sequences (the normal alpha-globin gene has | P | 1225.8 | A | 1205.1 | P | 2405 | A |
| | | two introns)/cds = (32,436)/gb = V00716/gi = 49902/ug = Mm 41983/len = 542/STRA = for | | | | | | | |
| 16 | 92558_at | Cluster Incl M84487 Vascular cell adhesion molecule 1/cds = (57,2276)/gb = M84487/gi = 202345/ug = Mm 1021/len = 3014/ | P | 105.1 | P | −1.5 | A | 116.3 | P |
| | | STRA = for | | | | | | | |
| 16 | 94861_at | Cluster Incl AA624073 vq71f12 s1 Mus musculus cDNA, 5 end/clone = IMAGE-1107791/clone_end = 5/gb = AA624073/ | P | 104.8 | A | 81.7 | A | 178.5 | P |
| | | gi = 2527949/ug = Mm.23682/len = 342/STRA = for | | | | | | | |
| 16 | 98005_at | Cluster Incl AW125442 UI-M-BH2 3-aqh-g-12-0-UI s1 Mus musculus cDNA, 3 end/clone = UI-M-BH2 3-aqh-g-12-0-UI/ | A | 130 | P | 101.7 | M | 150.5 | P |
| | | clone_end = 3/gb = AW125442/gi = 6100972/ug = Mm 3193/len = 487/STRA = for | | | | | | | |
| 16 | 101844_at | Cluster Incl U94700: Peroxisomal sarcosine oxidase/cds = (4,1176)/gb = U94700/gi = 2801410/ug = Mm 8543/len = 1686/ | P | 272.1 | A | 278.1 | A | 353.4 | A |
| | | STRA = for | | | | | | | |
| 16 | 102684_at | Cluster Incl U82375 Mus musculus MSG2alpha, beta, gamma, delta and epsilon salivary protein (Vcs2) genes, complete | P | 23.4 | P | 11.1 | A | 52.8 | A |
| | | cds/cds = (154,585)/gb = U82375/gi = 2289098/ug = Mm 1397/len = 743/STRA = for | | | | | | | |
| 16 | 92971_at | Cluster Incl AW125849 UI-M-BH2.2-aqk-d-08-0-UI.s1 Mus musculus cDNA, 3 end/clone = UI-M-BH2 2-aqk-d-08-0-UI/ | M | 104.4 | P | 89.3 | A | 158.5 | P |
| | | clone_end = 3/gb = AW125849/gi = 6101379/ug = Mm.5002/len = 468/STRA = rev | | | | | | | |
| 16 | 93463_at | Cluster Incl AW122517 UI-M-BH2.2-aox-a-10-0-UI s1 Mus musculus cDNA, 3 end/clone = UI-M-BH2 2-aox-a-10-0-UI/ | P | 75.5 | P | 104 | A | 111.4 | P |
| | | clone_end = 3/gb = AW122517/gi = 6098041/ug = Mm 69024/len = 439/STRA = rev | | | | | | | |
| 16 | 100634_at | Cluster Incl AF056194 Mus musculus short-chain dehydrogenase CRAD2 mRNA, complete cds/cds = (97,1047)/ | A | −68.8 | A | −253 | P | 17.9 | P |
| | | gb = AF056194/gi = 3294554/ug = Mm 6696/len = 1612/STRA = for | | | | | | | |
| 16 | 103085_at | Cluster Incl AB013095 Mus musculus mRNA for heme-binding protein, complete cds/cds = (57,629)/gb = AB013095/ | P | 220 | P | 143.4 | A | 424.1 | A |
| | | gi = 3724327/ug = Mm 17249/len = 1033/STRA = for | | | | | | | |
| 16 | 103804_at | Cluster Incl AB006960 Mus musculus mRECK mRNA, complete cds/cds = (6,2921)/gb = AB006960/gi = 3810870/ | A | 230.8 | P | 92.7 | A | 108.9 | P |
| | | ug = Mm 20862/len = 4094/STRA = for | | | | | | | |
| 16 | 95568_at | Cluster Incl AI853412.UI-M-BH0-ajy-a-05-0-UI s1 Mus musculus cDNA, 3 end/clone = UI-M-BH0-ajy-a-05-0-UI/ | A | 64 | P | 25.6 | A | 63 | P |
| | | clone_end = 3/gb = AI853412/gi = 5497318/ug = Mm 27799/len = 587/STRA = for | | | | | | | |
| 16 | 96810_at | Cluster Incl AI154017 ud58a11 r1 Mus musculus cDNA, 5 end/clone = IMAGE-1450076/clone_end = 5/gb = AI154017/ | A | 295.2 | P | 247 | A | 322.1 | P |
| | | gi = 3682486/ug = Mm 29266/len = 156/STRA = for | | | | | | | |
| 16 | 104402_at | Cluster Incl AA030469 mi23c07 r1 Mus musculus cDNA, 5 end/clone = IMAGE-464364/clone_end = 5/gb = AA030469/ | P | 96.3 | A | 61.1 | P | 105.4 | P |
| | | gi = 1497632/ug = Mm 2869/len = 342/STRA = for | | | | | | | |
| 16 | 93949_at | Cluster lncl M63658 M musculus G protein beta-subunit mRNA, complete cds/cds = (40,1062)/gb = M63658/gi = 193435/ | A | 68.8 | P | −28.9 | A | 135.5 | P |
| | | ug = Mm 9336/len = 1135/STRA = for | | | | | | | |
| 16 | 98829_at | Cluster Incl X80417 Potassium inwardly rectifying channel, subfamily J, member 12/cds = (1,1284)/gb = X80417/gi = 550388/ | A | 38.1 | P | 62.1 | P | 125.5 | P |
| | | ug = Mm 4970/len = 1618/STRA = for | | | | | | | |
| 16 | 102562_at | Cluster Incl Z80833 M musculus spermidine synthase pseudogene 2/cds = (0,852)/gb = Z80833/gi = 1592790/ug = Mm 57224/ | P | 568.6 | P | 515.1 | A | 559.8 | A |
| | | len = 853/STRA = for | | | | | | | |
| 16 | 95513_at | Cluster Incl AW124310 UI-M-BH2.1-apq-d-03-0-UI s1 Mus musculus cDNA, 3 end/clone = UI-M-BH2 1 -apq-d-03-0-UI/ | P | 201 | A | 125.8 | M | 194.3 | A |
| | | clone_end = 3/gb = AW124310/gi = 6099840/ug = Mm.25298/len = 234/STRA = for | | | | | | | |
| 16 | 95641_at | Cluster Incl AI853899 UI-M-BH0-aju-c-09-0-UI.s1 Mus musculus cDNA, 3 end/clone = UI-M-BH0-aju-c-09-0-UI/ | A | 239.1 | A | 278.7 | A | 756.1 | P |
| | | clone_end = 3/gb = AI853899/gi = 5497805/ug = Mm.29491/len = 380/STRA = for | | | | | | | |
| 16 | 103043_at | Cluster Incl Z35294 M musculus MTCP-1 gene/cds = (540,746)/gb = Z35294/gi = 603859/ug = Mm 16366/len = 954/ | A | 148 | P | −6.6 | A | 210.6 | P |
| | | STRA = for | | | | | | | |
| 16 | 103831_at | Cluster Incl U72194 Mus musculus muskelin mRNA, complete cds/cds = (12,2219)/gb = U72194/gi = 3493461/ | M | 128.2 | P | 110.6 | P | 172 | A |
| | | ug = Mm 20930/len = 3975/STRA = for | | | | | | | |
| 16 | 103869_at | Cluster Incl U16175 Mus musculus thrombospondin 3 (Thbs3) gene, partial cds and mucin 1 (Muc1) gene, complete cds/ | P | 150.3 | P | 58 | A | 209 | M |
| | | cds = (0,371)/gb = U16175/gi = 608488/ug = Mm 2114/len = 615/STRA = for | | | | | | | |
| 16 | 104042_at | Cluster Incl AI842275 UI-M-AM1-afz-d-12-0-UI s1 Mus musculus cDNA, 3 end/clone = UI-M-AM1-afz-d-12-0-UI/ | P | 160.2 | A | 161.4 | M | 254.1 | A |
| | | clone end = 3/gb = AI842275/gi = 5476528/ug = Mm 21470/len = 412/STRA = for | | | | | | | |
| 16 | 104752_at | Cluster Incl AB028863 Mus musculus mRNA for MMRP19, complete cds/cds = (5,730)/gb = AB028863/gi = 5103282/ | A | 423.5 | P | 264.3 | P | 524.3 | A |
| | | ug = Mm.24772/len = 840/STRA = for | | | | | | | |
| 16 | 94940_at | Cluster Incl AW123316 UI-M-BH2 1-apf-b-01-0-UI s1 Mus musculus cDNA, 3 end/clone = UI-M-BH2 1-apf-b-01-0-UI/ | A | 166.7 | P | 123.7 | P | 180.2 | M |
| | | clone_end = 3/gb = AW123316/gi = 6098846/ug = Mm.26925/len = 414/STRA = for | | | | | | | |
| 16 | 100968_at | Cluster Incl AW122609.UI-M-BH2 2-aol-a-11-0-UI s1 Mus musculus cDNA, 3 end/clone = UI-M-BH2 2-aol-a-11-0-UI/ | P | 118.7 | P | 105.4 | P | 136.8 | A |
| | | clone_end = 3/gb = AW122609/gi = 6098136/ug = Mm.6626/len = 257/STRA = rev | | | | | | | |
| 16 | 101939_at | Cluster Incl AI849344: UI-M-AJ1-aha-f-04-0-UI.s1 Mus musculus cDNA, 3 end/clone = UI-M-AJ1-aha-f-04-0-UI/ | A | 674.5 | P | 470.4 | A | 648.9 | A |
| | | clone_end = 3/gb = AI849344/gi = 5493250/ug = Mm.95187/len = 570/STRA = for | | | | | | | |
| 16 | 101940_at | Cluster Incl AJ222800: Mus musculus mRNA for neutral sphingomyelinase/cds = (309,1568)/gb = AJ222800/gi = 3021479/ | A | 216.4 | P | 198.6 | M | 269.4 | P |
| | | ug = Mm 953/len = 1627/STRA = for | | | | | | | |
| 16 | 103256_at | Cluster Incl AI509811: vx28b11 y1 Mus musculus cDNA, 5 end/clone = IMAGE-1265757/clone_end = 5/gb = AI509811/ | A | 315 | M | 172.8 | P | 289.8 | A |
| | | gi = 4408716/ug = Mm 2072/len = 251/STRA = for | | | | | | | |
| 16 | 92314_at | Cluster Incl U25652: Procollagen, type XII, alpha 1/cds = (83,9286)/gb = U25652/gi = 953236/ug = Mm 3819/len = 9704/ | A | 149.7 | P | 142.7 | A | 172.3 | P |
| | | STRA = rev | | | | | | | |
| 16 | 92482_at | Cluster Incl AF006466: Mus musculus lymphocyte specific formin related protein (Fr1) mRNA, complete cds/cds = (0,3194)/ | P | 143.7 | P | 182.2 | A | 271.6 | P |
| | | gb = AF006466/gi = 4101719/ug = Mm 42152/len = 3195/STRA = for | | | | | | | |
| 16 | 96509_at | Cluster Incl J05019 Fc receptor, IgE, high affinity I, beta polypeptide/cds = (22,729)/gb = J05019/gi = 193238/ug = Mm 2544/ | A | 125.5 | P | 158.1 | A | 174.9 | P |
| | | len = 1010/STRA = for | | | | | | | |
| 17 | AFFX- | E Coli BioB middle | P | 453.6 | P | 566.1 | P | 599.9 | P |
| | BioB-M_at | | | | | | | | |
| 17 | AFFX- | E Coli BioC 5 prime | P | 1053.4 | P | 1473.6 | P | 1502.7 | P |
| | BioC-5_at | | | | | | | | |
| 17 | AFFX- | E Coli BioD 5 prime | P | 1556.3 | P | 2035.1 | P | 1925.5 | P |
| | BioDn-5_at | | | | | | | | |
| 17 | 92554_at | Cluster Incl AF059735 Mus musculus C-terminal binding protein 2 CtBP2 mRNA, complete cds/cds = (0,1337)/ | P | 403.8 | P | 480.3 | P | 552.5 | P |
| | | gb = AF059735/gi = 3513570/ug = Mm.10172/len = 1338/STRA = rev | | | | | | | |
| 17 | 92593_at | Cluster Incl D13664 Mus musculus osf-2 mRNA for osteoblast specific factor 2, complete cds/cds = (18,2453)/gb = D13664/ | P | 4287.9 | P | 3365.9 | P | 4452.3 | P |
| | | gi = 393321/ug = Mm 10681/len = 3187/STRA = for | | | | | | | |
| 17 | 92771_at | Cluster Incl AB013357 Mus musculus mRNA for 49 kDa zinc finger protein, complete cds/cds = (110,1492)/gb = AB013357/ | P | 1770.7 | P | 1784 | P | 1820 | P |
| | | gi = 4153885/ug = Mm 12236/len = 2082/STRA = for | | | | | | | |
| 17 | 93067_f_at | Cluster Incl U62674 Mus musculus histone H2a 2-615 (H2a-615), and histone H3 2-615 (H3-615) genes, complete cds/ | P | 752.7 | P | 1251.4 | P | 1214.9 | P |
| | | cds = (43,435)/gb = U62674/gi = 1458142/ug = Mm.15686/len = 515/STRA = for | | | | | | | |
| 17 | 93070_at | Cluster Incl AI847564 UI-M-AM1-afv-g-08-0-UI s1 Mus musculus cDNA, 3 end/clone = UI-M-AM1-afv-g-08-0-UI/ | P | 492.4 | P | 459.1 | P | 588.1 | P |
| | | clone_end = 3/gb = AI847564/gi = 5491470/ug = Mm 15701/len = 348/STRA = for | | | | | | | |
| 17 | 93097_at | Cluster Incl U51805 Mus musculus liver arginase mRNA, complete cds/cds = (141,1112)/gb = U51805/gi = 1293092/ | P | 220.7 | P | 225.9 | P | 327.6 | P |
| | | ug = Mm 16423/len = 1221/STRA = for | | | | | | | |
| 17 | 93266_at | Cluster Incl U04541 Mus musculus alpha-tropomyosin slow mRNA, complete cds/cds = (53,910)/gb = U04541/gi = 439845/ | P | 906.1 | P | 1275.2 | P | 1139.4 | P |
| | | ug = Mm 17306/len = 1091/STRA = for | | | | | | | |
| 17 | 93325_at | Cluster Incl AI845735 UI-M-AQ1-aeb-b-07-0-UI s1 Mus musculus cDNA, 3 end/clone = UI-M-AQ1-aeb-b-07-0-UI/ | P | 259.9 | P | 349.2 | P | 418.5 | P |
| | | clone_end = 3/gb = AI845735/gi = 5489641/ug = Mm 18579/len = 362/STRA = for | | | | | | | |
| 17 | 93588_at | Cluster Incl Z54179 M musculus mRNA (evolutionary conserved transcript)/cds = (221,802)/gb = Z54179/gi = 1655636/ | P | 215.5 | P | 248.2 | P | 211.7 | P |
| | | ug = Mm 2080/len = 1217/STRA = for | | | | | | | |
| 17 | 93752_at | Cluster lncl AI848393: UI-M-AH1-ago-g-10-0-UI s1 Mus musculus cDNA, 3 end/clone = UI-M-AH1-ago-g-10-0-UI/ | P | 573 | P | 632.7 | P | 663.9 | P |
| | | clone_end = 3/gb = AI848393/gi = 5492294/ug = Mm 21118/len = 468/STRA = for | | | | | | | |
| 17 | 93768_f_at | Cluster Incl AW124052: UI-M-BH2 1-apn-e-06-0-UI s1 Mus musculus cDNA, 3 end/clone = UI-M-BH2 1-apn-e-06-0-UI/ | P | 720.5 | P | 799 | P | 966.2 | P |
| | | clone_end = 3/gb = AW124052/gi = 6099582/ug = Mm 21183/len = 246/STRA = for | | | | | | | |
| 17 | 93801 _at | Cluster Incl AA763275 vv89d02 r1 Mus musculus cDNA, 5 end/clone = IMAGE-1229571/clone_end = 5/gb = AA763275/ | P | 2760.4 | P | 4833.6 | P | 4027.7 | P |
| | | gi = 2813022/ug = Mm 21408/len = 357/STRA = for | | | | | | | |
| 17 | 94044_at | Cluster Incl AA692708 vr54d07 s1 Mus musculus cDNA, 5 end/clone = IMAGE-1124461/clone_end = 5/gb = AA692708/ | P | 342 | P | 457.4 | P | 419.3 | P |
| | | gi = 2693646/ug = Mm 21964/len = 170/STRA = for | | | | | | | |
| 17 | 94232_at | Cluster Incl AI849928: UI-M-BG0-ahz-e-10-0-UI s1 Mus musculus cDNA, 3 end/clone = UI-M-BG0-ahz-e-10-0-UI/ | P | 974.2 | P | 1456.6 | P | 1423.9 | P |
| | | clone_end = 3/gb = AI849928/gi = 5493834/ug = Mm 22288/len = 482/STRA = for | | | | | | | |
| 17 | 94295_at | Cluster Incl AF017085 Mus musculus BAP-135 homolog (Diws1t) mRNA, complete cds/cds = (87,3023)/gb = AF017085/ | P | 1130.5 | P | 1522.3 | P | 1395.9 | P |
| | | gi = 2653820/ug = Mm 22593/len = 4091/STRA = for | | | | | | | |
| 17 | 94296_s_at | Cluster Incl AF043220 Mus musculus TFII-I protein short form mRNA, alternatively spliced, complete cds/cds = (4,2880)/ | P | 196.4 | P | 236.3 | P | 302.3 | P |
| | | gb = AF043220/gi = 2895589/ug = Mm 22593/len = 2936/STRA = for | | | | | | | |
| 17 | 96041_at | Cluster Incl AB016424 Mus musculus rbm3 mRNA, complete cds/cds = (85,546)/gb = AB016424/gi = 3810891/ug = Mm 2591/ | P | 4947.1 | P | 5846.2 | P | 6206.1 | P |
| | | len = 1158/STRA = for | | | | | | | |
| 17 | 96104_at | Cluster Incl AI047107 uh58b11 r1 Mus musculus cDNA, 5 end/clone = IMAGE-1749597/clone_end = 5/gb = AI047107/ | P | 466 | P | 405.2 | P | 486.3 | P |
| | | gi = 3295394/ug = Mm 26879/len = 403/STRA = for | | | | | | | |
| 17 | 96649_at | Cluster Incl AW046552 UI-M-BH1-alb-b-06-0-UI.s1 Mus musculus cDNA, 3 end/clone = UI-M-BH1-alb-b-06-0-UI/ | P | 320.7 | P | 365.5 | P | 366.6 | P |
| | | clone_end = 3/gb = AW046552/gi = 5907081/ug = Mm 27328/len = 517/STRA = for | | | | | | | |
| 17 | 97857_at | Cluster Incl AW047762 UI-M-BH1-alk-d-04-0-UI s1 Mus musculus cDNA, 3 end/clone = UI-M-BH1-alk-d-04-0-UI/ | P | 235.3 | P | 324.3 | P | 381.2 | P |
| | | clone_end = 3/gb = AW047762/gi = 5908291/ug = Mm 28300/len = 387/STRA = for | | | | | | | |
| 17 | 97888_at | Cluster Incl AI849676 UI-M-AL1-ahl-e-06-0-UI.s1 Mus musculus cDNA, 3 end/clone = UI-M-AL1-ahl-e-06-0-UI/ | P | 262.9 | P | 412.4 | P | 430.5 | P |
| | | clone_end = 3/gb = AI849676/gi = 5493582/ug = Mm.28397/len = 367/STRA = for | | | | | | | |
| 17 | 97893_at | Cluster Incl AB017697 Mus musculus TLP21 mRNA for 21-kDa TBP-like protein, complete cds/cds = (272,832)/ | P | 121.3 | P | 124.4 | P | 166.3 | P |
| | | gi = AB017697/gi = 4218069/ug = Mm 28415/len = 1273/STRA = for | | | | | | | |
| 17 | 97908_at | Cluster Incl AW120676 UI-M-BH2 3-anz-b-10-0-UI.s1 Mus musculus cDNA, 3 end/clone = UI-M-BH2.3-anz-b-10-0-UI/ | P | 1201.1 | P | 1073.6 | P | 1709 | P |
| | | clone_end = 3/gb = AW120676/gi = 6095974/ug = Mm.26474/len = 452/STRA = for | | | | | | | |
| 17 | 98924_at | Cluster Incl Y08027 M musculus ART3 gene/cds = (0,1112)/gb = Y08027/gi = 2677615/ug = Mm 28637/len = 1113/STRA = for | P | 130.1 | P | 151 | P | 291.3 | P |
| 17 | 94537_at | Cluster Incl AI838859 UI-M-AN0-ack-c-06-0-UI s1 Mus musculus cDNA, 3 end/clone = UI-M-AN0-ack-c-06-0-UI/ | P | 1824.6 | P | 2476.6 | P | 2028.8 | P |
| | | clone_end = 3/gb = AI838859/gi = 5473072/ug = Mm 29057/len = 349/STRA = for | | | | | | | |
| 17 | 94542_at | Cluster Incl AA929348: vz41b07.r1 Mus musculus cDNA, 5 end/clone = IMAGE-1329013/clone_end = 5/gb = AA929348/ | P | 187.9 | P | 239.8 | P | 265.3 | P |
| | | gi = 3078657/ug = Mm 29087/len = 166/STRA = for | | | | | | | |
| 17 | 95056_r_at | Cluster Incl AW122747 UI-M-BH2.2-aot-g-01-0-UI s1 Mus musculus cDNA, 3 end/clone = UI-M-BH2 2-aot-g-01-0-UI/ | P | 114.7 | P | 169.2 | P | 174.1 | P |
| | | clone_end = 3/gb = AW122747/gi = 6098277/ug = Mm 29150/len = 468/STRA = for | | | | | | | |
| 17 | 95078_at | Cluster Incl AI841400 UI-M-AH0-acx-a-06-0-UI.s1 Mus musculus cDNA, 3 end/clone = UI-M-AH0-acx-a-06-0-UI/ | P | 331.3 | P | 254.8 | P | 351.6 | P |
| | | clone_end = 3/gb = AI841400/gi = 5475613/ug = Mm 29213/len = 213/STRA = for | | | | | | | |
| 17 | 95092_at | Cluster Incl J05479: Mouse calcineurin catalytic subunit mRNA, complete cds/cds = (75,1640)/gb = J05479/gi = 192347/ | P | 594.5 | P | 803.9 | P | 745.4 | P |
| | | ug = Mm.293/len = 2194/STRA = for | | | | | | | |
| 17 | 95142_s_at | Cluster lncl U10407 Capping protein beta 1/cds = (18,836)/gb = U10407/gi = 500748/ug = Mm 2945/len = 1014/STRA = for | P | 331.4 | P | 909.6 | P | 1487.5 | P |
| 17 | 95646_at | Cluster Incl U01170 Camiline palmitoyltransferase 2/cds = (0,1976)/gb = U01170/gi = 425266/ug = Mm 29499/len = 1977/ | P | 775.7 | P | 701.2 | P | 873.3 | P |
| | | STRA = for | | | | | | | |
| 17 | 95730_at | Cluster Incl AI840884 UI-M-AM0-adm-b-06-0-UI s1 Mus musculus cDNA, 3 end/clone = UI-M-AM0-adm-b-06-0-UI/ | P | 710.5 | P | 862.2 | P | 972.2 | P |
| | | clone_end = 3/gb = AI840884/gi = 5475097/ug = Mm 29741/len = 317/STRA = for | | | | | | | |
| 17 | 96299_at | Cluster Incl AIB47298: UI-M-AN1-afh-c-08-0-UI s1 Mus musculus cDNA, 3 end/clone = UI-M-AN1-afh-c-08-0-UI/ | P | 2000.4 | P | 2117 | P | 2187.7 | P |
| | | clone_end = 3/gb = AI847298/gi = 5491204/ug = Mm 29909/len = 328/STRA = for | | | | | | | |
| 17 | 96322_at | Cluster Incl AI836001 UI-M-AJ0-abb-b-07-0-UI s1 Mus musculus cDNA, 3 end/clone = UI-M-AJ0-abb-b-07-0-UI/ | P | 634.1 | P | 797.5 | P | 745.8 | P |
| | | clone_end = 3/gb = AI836001/gi = 5470254/ug = Mm 29945/len = 481/STRA = for | | | | | | | |
| 17 | 97518_at | Cluster Incl D29016 Mouse mRNA for squalene synthase/cds = (20,1270)/gb = D29016/gi = 468456/ug = Mm 3204/len = 1634/ | P | 495.4 | P | 615.5 | P | 777.1 | P |
| | | STRA/ = for | | | | | | | |
| 17 | 98039_at | Cluster Incl AI853819 UI-M-BH0-ajr-a-08-0-UI s1 Mus musculus cDNA, 3 end/clone = UI-M-BH0-ajr-a-08-0-UI/ | P | 531 | P | 912.3 | P | 867.9 | P |
| | | clone_end = 3/gb = AI853819/gi = 5497725/ug = Mm 34002/len = 369/STRA = for | | | | | | | |
| 17 | 98138_at | Cluster Incl AI854776 UI-M-BH0-aka-d-05-0-UI s1 Mus musculus cDNA, 3 end/clone = UI-M-BH0-aka-d-05-0-UI/ | P | 305.1 | P | 432.4 | P | 424.5 | P |
| | | clone_end = 3/gb = AI854776/gi = 5498682/ug = Mm.35538/len = 333/STRA = for | | | | | | | |
| 17 | 98604_at | Cluster Incl U79774 Mus musculus NNP-1 var (NNP-1) mRNA, complete cds/cds = (43,1527)/gb = U79774/gi = 2258277/ | P | 434.4 | P | 451.2 | P | 459.8 | P |
| | | ug = Mm 38344/len = 1946/STRA = for | | | | | | | |
| 17 | 99102_at | Cluster Incl U67874.Ubiquitin specific protease 9, X chromosome/cds = (355,8034)/gb = U67874/gi = 1527198/ug = Mm 396/ | P | 81.4 | P | 82.1 | P | 89 | P |
| | | len = 10323/STRA = for | | | | | | | |
| 17 | 99121_at | Cluster Incl X90875 M musculus mRNA for FXR1 protein/cds = (13,1632)/gb = X90875/gi = 1122421/ug = Mm 4038/ | P | 231.5 | P | 232.9 | P | 271.8 | P |
| | | len = 2060/STRA = for | | | | | | | |
| 17 | 99148_at | Cluster Incl AI852862 UI-M-BH0-ajk-e-02-0-UI s1 Mus musculus cDNA, 3 end/clone = UI-M-BH0-ajk-e-02-0-UI/ | P | 981.8 | P | 1467.9 | P | 1414.2 | P |
| | | clone_end = 3/gb = AI852862/gi = 5496768/ug = Mm.41502/len = 266/STRA = for | | | | | | | |
| 17 | 99184_at | Cluster Incl AW120896 UI-M-BH2.3-aob-b-10-0-UI s1 Mus musculus cDNA, 3 end/clone = UI-M-BH2 3-aob-b-10-0-UI/ | P | 673.8 | P | 932.8 | P | 1047.9 | P |
| | | clone_end = 3/gb = AW120896/gi = 6096229/ug = Mm 41853/len = 302/STRA = for | | | | | | | |
| 17 | 99580_s_at | Cluster Incl U16818 Mus musculus UDP glucuronosyltransferase (UGT1-06) mRNA, complete cds/cds = (128,1723)/ | P | 1443.1 | P | 1509.9 | P | 1362.5 | P |
| | | gb = U16818/gi = 801898/ug = Mm 42472/len = 2300/STRA = for | | | | | | | |
| 17 | 99582_at | Cluster Incl M76124 Mus musculus EGP314 precursor mRNA, complete cds/cds = (107,1051)/gb = M76124/gi = 193007/ | P | 137.7 | P | 137.6 | P | 165 | P |
| | | ug = Mm 4259/len = 1710/STRA = for | | | | | | | |
| 17 | 99666_at | Cluster Incl AW125431: UI-M-BH2.3-aqh-g-01-0-UI s1 Mus musculus cDNA, 3 end/clone = UI-M-BH2 3-aqh-g-01-0-UI/ | P | 2237.5 | P | 2341.1 | P | 2651.5 | P |
| | | clone_end = 3/gb = AW125431/gi = 6100961/ug = Mm 43822/len = 406/STRA = for | | | | | | | |
| 17 | 100587_f_at | Cluster Incl AI843959 UI-M-AP1-agh-e-01-0-UI s1 Mus musculus cDNA, 3 end/clone = UI-M-AP1-agh-e-01-0-UI/ | P | 242.4 | P | 215.8 | P | 250.8 | P |
| | | clone_end = 3/gb = AI843959/gi = 5478172/ug = Mm 622/len = 379/STRA = for | | | | | | | |
| 17 | 101557_at | Cluster Incl AF043070 Mus musculus branched chain alpha ketoacid dehydrogenase kinase (bckd-kinase) mRNA, nuclear | P | 1242.3 | P | 1330.2 | P | 1330.9 | P |
| | | gene encoding mitochondrial protein, complete cds/cds = (106,1344)/gb = AF043070/gi = 2809219/ug = Mm.8903/len = 1654/ | | | | | | | |
| | | STRA = for | | | | | | | |
| 17 | 101564_at | Clusler Incl U21855 Mus musculus mCAF1 protein mRNA, complete cds/cds = (39,896)/gb = U21855/gi = 726135/ | P | 187 | P | 151.5 | P | 200.6 | P |
| | | ug = Mm 89351/len = 1264/STRA = for | | | | | | | |
| 17 | 101962_at | Cluser Incl AI854884: UI-M-BH0-akb-f-11-0-UI s1 Mus musculus cDNA, 3 end/clone = UI-M-BH0-akb-f-11-0-UI/ | P | 1168.2 | P | 1366.1 | P | 1532.6 | P |
| | | clone_end = 3/gb = AI854884/gi = 5498790/ug = Mm 92820/len = 407/STRA = for | | | | | | | |
| 17 | 101990_at | Cluster Incl X51905: Lactate dehydrogenase 2, B chain/cds = (46,1050)/gb = X51905/gi = 52879/ug = Mm 9745/len = 1238/ | P | 301.6 | P | 398 | P | 439.3 | P |
| | | STRA = for | | | | | | | |
| 17 | 102018_at | Cluster Incl AI854879 UI-M-BH0-akb-f-05-0-UI.s1 Mus musculus cDNA, 3 end/clone = UI-M-BH0-akb-f-05-0-UI/ | P | 297.5 | P | 343.2 | P | 339.3 | P |
| | | clone_end = 3/gb = AI854879/gi = 5498785/ug = Mm 10038/len = 127/STRA = for | | | | | | | |
| 17 | 102054_at | Cluster Incl AB011370.Mus musculus Ankhzn mRNA, complete cds/cds = (21,3575)/gb = AB011370/gi = 2914016/ | P | 400.2 | P | 392.6 | P | 385.9 | P |
| | | ug = Mm 10313/len = 7143/STRA = for | | | | | | | |
| 17 | 102060_at | Cluster Incl AF051357 Mus musculus golgin-245 (olp-1) mRNA, complete cds/cds = (87,3743)/gb = AF051357/gi = 2952521/ | P | 537.4 | P | 492.2 | P | 605.4 | P |
| | | ug = Mm 10409/len = 4393/STRA = rev | | | | | | | |
| 17 | 102812_I_at | Cluster Incl AW210346 uI53g12 y1 Mus musculus cDNA, 5 end/clone = IMAGE-2102086/clone_end = 5/gb = AW210346/ | P | 420.5 | P | 413.3 | P | 470.6 | P |
| | | gi = 6516286/ug = Mm.14253/len = 431/STRA = for | | | | | | | |
| 17 | 103352_at | Cluster Incl X65603 Dolichyl-phosphate alpha-N-acetylglucosaminephosphotransferase 2/cds = (59,1291)/gb = X65603/ | P | 220.1 | P | 237.2 | P | 331.5 | P |
| | | gi = 51077/ug = Mm 18353/len = 1764/STRA = for | | | | | | | |
| 17 | 103421_at | Cluster Incl D50464.Mouse SDR2 mRNA, complete cds/cds = (128,1906)/gb = D50464/gi = 1747305/ug = Mm.18910/ | P | 168.7 | P | 291 | P | 228.8 | P |
| | | len = 2394/STRA = for | | | | | | | |
| 17 | 103424_at | Cluster Incl AI844839 UI-M-BG0-ahv-a-06-0-UI s1 Mus musculus cDNA, 3 end/clone = UI-M-BG0-ahv-a-06-0-UI/ | P | 394.7 | P | 401.9 | P | 567.6 | P |
| | | clone_end = 3/gb = AI844839/gi = 5488785/ug = Mm 18957/len = 423/STRA = for | | | | | | | |
| 17 | 103429_i_at | Cluster Incl AW125330 UI-M-BH2 1-apy-e-12-0-UI s1 Mus musculus cDNA, 3 end/clone = UI-M-BH2 1-apy-e-12-0-UI/ | P | 115.4 | P | 120.7 | P | 128.1 | P |
| | | clone_end = 3/gb = AW125330/gi = 6100855/ug = Mm 19006/len = 208/STRA = for | | | | | | | |
| 17 | 103567_at | Cluster Incl AW123810 UI-M-BH2 1-apm-f-05-0-UI s1 Mus musculus cDNA, 3 end/clone = UI-M-BH2.1-apm-f-05-0-UI/ | P | 663.8 | P | 655.6 | P | 713.6 | P |
| | | clone_end = 3/gb = AW123810/gi = 6099340/ug = Mm 1921/len = 404/STRA = for | | | | | | | |
| 17 | 103585_at | Cluster Incl AI851408 UI-M-BH0-akl-b-03-0-UI s1 Mus musculus cDNA, 3 end/clone = UI-M-BH0-alq-b-03-0-UI/ | P | 204.5 | P | 204.3 | P | 223.2 | P |
| | | clone_end = 3/gb = AI851408/gi = 5495314/ug = Mm 19804/len = 342/STRA = for | | | | | | | |
| 17 | 103648_at | Cluster Incl Y08830 M musculus trop2 gene/cds = (0,953)/gb = Y08830/gi = 2764995/ug = Mm.20410/len = 954/STRA = for | P | 1266.3 | P | 1560.4 | P | 1918.2 | P |
| 17 | 103672_at | Cluster Incl AW122572 UI-M-BH2 2-aox-f-06-0-UI s1 Mus musculus cDNA, 3 end/clone = UI-M-BH2 2-aox-f-06-0-UI/ | P | 419.3 | P | 431.3 | P | 496.7 | P |
| | | clone_end = 3/gb = AW122572/gi = 6098097/ug = Mm20805/len = 505/STRA = for | | | | | | | |
| 17 | 103845_at | Cluster Incl AI839005 UI-M-AN0-acm-c-06-0-UI s1 Mus musculus cDNA, 3 end/clone = UI-M-AN0-acm-c-06-0-UI/ | P | 458 | P | 517.1 | P | 471.2 | P |
| | | clone_end = 3/gb = AI839005/gi = 5473218/ug = Mm 2097/len = 447/STRA = rev | | | | | | | |
| 17 | 103912_at | Cluster Incl AW046563: UI-M-BH1-alb-c-08-0-UI.s1 Mus musculus cDNA, 3 end/clone = UI-M-BH1-alb-c-08-0-UI/ | P | 449 | P | 537 | P | 531.2 | P |
| | | clone_end = 3/gb = AW046563/gi = 5907092/ug = Mm.21428/len = 520/STRA = for | | | | | | | |
| 17 | 104050_at | Cluster Incl AI846060: UI-M-AK1-aew-g-02-0-UI s1 Mus musculus cDNA, 3 end/clone = UI-M-AK1-aew-g-02-0-UI/ | P | 1096.7 | P | 1130 | P | 1085.8 | P |
| | | clone_end = 3/gb = AI846060/gi = 5490006/ug = Mm 21515/len = 229/STRA = rev | | | | | | | |
| 17 | 104125_at | Cluster Incl AA763673 vp09g09 r1 Mus musculus cDNA, 5 end/clone = IMAGE-1068160/clone_end = 5/gb = AA763673/ | P | 509 | P | 565.6 | P | 880.3 | P |
| | | gi = 2813755/ug = Mm 22030/len = 568/STRA = for | | | | | | | |
| 17 | 104337_f_at | Cluster Incl AW208938 uo65g07 x1 Mus musculus cDNA, 3 end/clone = IMAGE-2647452/clone_end = 3/gb = AW208938/ | P | 232 | P | 296.6 | P | 361.2 | P |
| | | gi = 6514878/ug = Mm 2252/len = 715/STRA = for | | | | | | | |
| 17 | 104340_at | Cluster Incl AF072240 Mus musculus methyl-CpG binding protein MBD1(Mbd1) mRNA, complete cds/cds = (175,2085)/ | P | 104.2 | P | 125.7 | P | 156 | P |
| | | gb = AF072240/gi = 3800788/ug = Mm 22522/len = 2820/STRA = for | | | | | | | |
| 17 | 104522_at | Cluster Incl AI047617: Uh63h03.r1 Mus musculus cDNA, 5 end/clone = IMAGE-1750133/clone_end = 5/gb = AI047617/ | P | 257.4 | P | 283.1 | P | 347 | P |
| | | gi = 3295904/ug = Mm 22930/len = 344/STRA = for | | | | | | | |
| 17 | 104557_at | Cluster Incl AI847097 UI-M-AP1-agn-f-01-0-UI s1 Mus musculus cDNA, 3 end/clone = UI-M-AP1-agn-f-01-0-UI/ | P | 646.5 | P | 579.9 | P | 755.8 | P |
| | | clone_end = 3/gb = AI847097/gi = 5491003/ug = Mm 23807/len = 203/STRA = rev | | | | | | | |
| 17 | 104577_at | Cluster Incl AA920621 vz07106 r1 Mus musculus cDNA, 5 end/clone = IMAGE-1315043/clone_end = 5/gb = AA920621/ | P | 112.1 | P | 130.4 | P | 175.7 | P |
| | | gi = 3067400/ug = Mm 23949/len = 294/STRA = for | | | | | | | |
| 17 | 104582_g_at | Cluster Incl AI845438 UI-M-AO1-aej-d-10-0-UI s1 Mus musculus cDNA, 3 end/clone = UI-M-AO1-aej-d-10-0-UI/ | P | 131.7 | P | 166.5 | P | 196.7 | P |
| | | clone_end = 3/gb = AI845438/gi = 5489339/ug = Mm 23975/len = 369/STRA = for | | | | | | | |
| 17 | 104589_at | Cluster Incl AF091096 Mus musculus NNX3 mRNA, complete cds/cds = (76,1449)/gb = AF091096/gi = 4165096/ | P | 739.2 | P | 1061.6 | P | 1089.2 | P |
| | | ug = Mm 23997/len = 2044/STRA = for | | | | | | | |
| 17 | 104604_at | Cluster Incl U62908 Mus musculus zinc linger protein 96 (Zfp96) mRNA, complete cds/cds = (432,1937)/gb = U62908/ | P | 80.8 | P | 119.2 | P | 127.2 | P |
| | | gi = 4097500/ug = Mm.24124/len = 2307/STRA = for | | | | | | | |
| 17 | 104611_at | Cluster Incl AI854008 UI-M-BH0-arv-h-12-0-UI s1 Mus musculus cDNA, 3 end/clone = UI-M-BH0-arv-h-12-0-UI/ | P | 323.8 | P | 337.1 | P | 428.5 | P |
| | | clone_end = 3/gb = AI854008/gi = 5497914/ug = Mm 24158/len = 494/STRA = for | | | | | | | |
| 17 | 94352_at | Cluster Incl AI850675 UI-M-BG1-ail-a-01-0-UI s1 Mus musculus cDNA, 3 end/clone = UI-M-BG1-ail-a-01-0-UI/ | P | 307.1 | P | 354.6 | P | 605 | P |
| | | clone_end = 3/gb = AI850675/gi = 5494581/ug = Mm 25200/len = 215/STRA = for | | | | | | | |
| 17 | 94426_at | Cluster Incl AI851052 UI-M-BH0-ajv-g-02-0-UI s1 Mus musculus cDNA, 3 end/clone = UI-M-BH0-ajv-g-02-0-UI/ | P | 556.3 | P | 694.7 | P | 822.8 | P |
| | | clone_end = 3/gb = AI851052/gi = 5494958/ug = Mm.26392/len = 404/STRA = for | | | | | | | |
| 17 | 95528_at | Cluster Incl AA791234 vv92e10 r1 Mus musculus cDNA, 5 end/clone = IMAGE-1229898/clone_end = 5/gb = AA791234/ | P | 524.8 | P | 911.5 | P | 833.7 | P |
| | | gi = 2851354/ug = Mm 27613/len = 347/STRA = for | | | | | | | |
| 17 | 95571_at | Cluster Incl AF004100: Zinc transporter 4/cds = (0,600)/gb = AF004100/gi = 2580599/ug = Mm 27801/len = 620/STRA = rev | P | 184.1 | P | 120.8 | P | 210.2 | P |
| 17 | 95577_at | Custer Incl AW049625 UI-M-BH1-and-e-07-0-UI s1 Mus musculus cDNA, 3 end/clone = UI-M-BH1-and-e-07-0-UI/ | P | 491.8 | P | 519.1 | P | 704 | P |
| | | clone_end = 3/gb = AW049625/gi = 5910154/ug = Mm 27829/len = 352/STRA = rev | | | | | | | |
| 17 | 95616_at | Cluster Incl AA674714 vm74b12 s1 Mus musculus cDNA, 5 end/clone = IMAGE-1003967/clone_end = 5/gb = AA674714/ | P | 117 | P | 103.3 | P | 117.5 | P |
| | | gi = 2651951/ug = Mm 28020/len = 156/STRA = for | | | | | | | |
| 17 | 95637_at | Cluster Incl AI838592.UI-M-AO0-abz-e-02-0-UI s1 Mus musculus cDNA, 3 end/clone = UI-M-AO0-abz-e-02-0-UI/ | P | 342.5 | P | 445.3 | P | 437.2 | P |
| | | clone_end = 3/gb = AI838592/gi = 5472805/ug = Mm 28095/len = 321/STRA = for | | | | | | | |
| 17 | 96144_at | Cluster Incl AJ001972 Mus musculus Id4 dominant negative helix-loop-helix gene/cds = (0,485)/gb = AJ001972/gi = 2546999/ | P | 567.3 | P | 706.3 | P | 644.9 | P |
| | | ug = Mm 28223/len = 486/STRA = for | | | | | | | |
| 17 | 96151_at | Cluster Incl AA839813 vw52c02 r1 Mus musculus cDNA, 5 end/clone = IMAGE-1247426/clone_end = 5/gb = AA839813/ | P | 276.7 | P | 375.9 | P | 455.7 | P |
| | | gi = 2916407/ug = Mm 28252/len = 527/STRA = for | | | | | | | |
| 17 | 96166_at | Cluster Incl AI842524 UI-M-AQ1-aea-c-06-0-UI s1 Mus musculus cDNA, 3 end/clone = UI-M-AQ1-aea-c-06-0-UI/ | P | 597.7 | P | 631.3 | P | 737.4 | P |
| | | clone_end = 3/gb = AI842524/gi = 5476737/ug = Mm 28371/len = 440/STRA = for | | | | | | | |
| 17 | 96217_at | Cluster Incl AW060552: UI-M-BH1-ann-g-10-0-UI.s1 Mus musculus cDNA, 3 end/clone = UI-M-BH1-ann-g-10-0-UI/ | P | 278.8 | P | 258.8 | P | 309.1 | P |
| | | clone_end = 3/gb = AW060552/gi = 6008303/ug = Mm 28684/len = 334/STRA = for | | | | | | | |
| 17 | 96724_r_at | Cluster Incl AW122911 UI-M-BH2 1-apa-f-09-0-UI.s1 Mus musculus cDNA, 3 end/clone = UI-M-BH2 1-apa-f-09-0-UI/ | P | 438.7 | P | 493.3 | P | 477.4 | P |
| | | clone_end = 3/gb = AW122911/gi = 6098441/ug = Mm 28829/len = 366/STRA = for | | | | | | | |
| 17 | 96734_at | Cluster Incl AA674143 vp96d10 r1 Mus musculus cDNA, 5 end/clone = IMAGE-1092595/clone_end = 5/gb = AA674143/ | P | 491.9 | P | 462.5 | P | 587.5 | P |
| | | gi-2651380/ug = Mm.28894/len = 474/STRA = for | | | | | | | |
| 17 | 96736_at | Cluster Incl AI850370 UI-M-BG1-aig-c-02-0-UI s1 Mus musculus cDNA, 3 end/clone = UI-M-BG1-aig-c-02-0-UI/ | P | 881.2 | P | 1136.7 | P | 1162 | P |
| | | clone_end = 3/gb = AI850370/gi = 5494276/ug = Mm 28900/len = 432/STRA = rev | | | | | | | |
| 17 | 96750_at | Cluster Incl AW122179 UI-M-BH2 2-aov-a-01-0-UI s1 Mus musculus cDNA, 3 end/clone = UI-M-BH2 2-aov-a-01-0-UI/ | P | 387.7 | P | 305.7 | P | 442.3 | P |
| | | clone_end = 3/gb = AW122179/gi = 6097626/ug = Mm 28965/len = 496/STRA = for | | | | | | | |
| 17 | 96781_at | Cluster Incl AI838293.UI-M-AO0-aby-c-04-0-UI s1 Mus musculus cDNA, 3 end/clone = UI-M-AO0-aby-c-04-0-UI/ | P | 394 | P | 452.4 | P | 476.2 | P |
| | | clone_end = 3/gb = AI838293/gi = 5472506/ug = Mm 29072/len = 310/STRA = for | | | | | | | |
| 17 | 98020_at | Cluster Incl D10715.Mouse mRNA for novel GTP-binding protein, complete cds/cds = (76,1179)/gb = D10715/gi = 220506/ | P | 782.6 | P | 1245.4 | P | 1241.8 | P |
| | | ug = Mm.3250/len = 1524/STRA = for | | | | | | | |
| 17 | 98032_at | Cluster Incl M36146 Zinc finger protein 35/cds = (164,1906)/gb = M36146/gi = 202451/ug = Mm.3297/len = 2293/STRA = for | P | 200.7 | P | 265.5 | P | 343.8 | P |
| 17 | 98397_at | Cluster Incl AI852694 UI-M-BH0-aji-d-08-0-UI.s1 Mus musculus cDNA, 3 end/clone = UI-M-BH0-aji-d-08-0-UI/clone_end = 3/ | P | 296.7 | P | 246.8 | P | 296.3 | P |
| | | gb = AI852694/gi = 5496600/ug = Mm 33285/len = 495/STRA = for | | | | | | | |
| 17 | 98464_at | Cluster Incl AW124196 UI-M-BH2 1-aph-b-03-0-UI s1 Mus musculus cDNA, 3 end/clone = UI-M-BH2 1-aph-b-03-0-UI/ | P | 323.9 | P | 346 | P | 350.5 | P |
| | | clone_end = 3/gb = AW124196/gi = 6099726/ug = Mm 34812/len 382/STRA = rev | | | | | | | |
| 17 | 98486_at | Cluster Incl AF105222 Mus musculus slathmin-like-protein RB3 mRNA, complete cds/cds = (88,657)/gb = AF105222/ | P | 375 | P | 593.2 | P | 715.1 | P |
| | | gi = 4028597/ug = Mm 35474/len = 1197/STRA = rev | | | | | | | |
| 17 | 98982_at | Cluster Incl AW046443 UI-M-BH1-akp-e-04-0-UI s1 Mus musculus cDNA, 3 end/clone = UI-M-BH1-akp-e-04-0-UI/ | P | 771 | P | 835.3 | P | 1138.9 | P |
| | | clone_end = 3/gb = AW046443/gi = 5906972/ug = Mm.36912/len = 439/STRA = for | | | | | | | |
| 17 | 99485_at | Cluster Incl AB009376 Mus musculus mRNA for ICAD-S, complete cds/cds = (0,797)/gb = AB009376/gi = 2754587/ | P | 240.7 | P | 330.4 | P | 346.9 | P |
| | | ug = Mm 41433/len = 798/STRA = for | | | | | | | |
| 17 | 99536_at | Cluster Incl AB016080 Mus musculus mRNA for calcium binding protein Ktp 2, complete cds/cds = (61,624)/gb = AB016080/ | P | 179 | P | 257 | P | 263.6 | P |
| | | gi = 4115809/ug = Mm.42192/len = 736/STRA = for | | | | | | | |
| 17 | 99549_at | Cluster Incl D31951: Mouse mRNA for osteoglycin, complete cds/cds = (39,935)/gb = D31951/gi = 971273/ug = Mm.4258/ | P | 152.8 | P | 164.5 | P | 199 | P |
| | | len = 1268/STRA = for | | | | | | | |
| 17 | 99975_at | Cluster Incl AA879937: vw02b10.r1 Mus musculus cDNA, 5 end/clone = IMAGE-1230715/clone_end = 5/gb = AA879937/ | P | 360.7 | P | 391.7 | P | 523.1 | P |
| | | gi = 2988920/ug = Mm 4428/len = 331/STRA = for | | | | | | | |
| 17 | 100009_r_at | Cluster Incl X94127 SRY-box containing gene 2/cds = (0,959)/gb = X94127/gi = 1209429/ug = Mm 4541/len = 960/STRA = for | P | 78.9 | P | 112.4 | P | 96.9 | P |
| 17 | 100496_at | Cluster Incl U79523 Mus musculus peptidylglycine alpha-amidating monooxygenase (PAM) mRNA, complete cds/ | P | 433.9 | P | 547.5 | P | 511.0 | P |
| | | cds = (335,3274)/gb = U79523/gi = 1711198/ug = Mm.5121/len = 4032/STRA = for | | | | | | | |
| 17 | 101877_at | Cluster Incl AI854432 UI-M-BH0-ail-c-06-0-UI s1 Mus musculus cDNA, 3 end/clone = UI-M-BH0-ail-c-06-0-UI/clone_end = 3/ | P | 502.5 | P | 557.5 | P | 526 | P |
| | | gb = AI854432/gi = 5498338/ug = Mm 88182/len = 413/STRA = rev | | | | | | | |
| 17 | 101912_at | Cluster Incl AI019679 ua92e01.r1 Mus musculus cDNA, 5 end/clone = IMAGE-1364952/clone_end = 5/gb = AI019679/ | P | 1805.5 | P | 2207.3 | P | 2707.3 | P |
| | | gi = 3234015/ug = Mm 902/len = 363/STRA = for | | | | | | | |
| 17 | 102215_at | Cluster Incl Y10495 Mus musculus mRNA for CDV-1R protein/cds = (886,2130)/gb = Y10495/gi = 2995446/ug = Mm 10125/ | P | 229.7 | P | 220.6 | P | 276.8 | P |
| | | len = 2649/STRA = for | | | | | | | |
| 17 | 102894_g_at | Cluster Incl X68363 POU domain, class 2, transcription factor 1/cds = (52,2292)/gb = X68363/gi = 53471/ug = Mm 15568/ | P | 150.5 | P | 356 | P | 451.9 | P |
| | | len = 2574/STRA = for | | | | | | | |
| 17 | 102980_at | Cluster Incl AI849119 UI-M-AJ1-agy-a-03-0-UI s1 Mus musculus cDNA, 3 end/clone = UI-M-AJ1-agy-a-03-0-UI/ | P | 303.5 | P | 471.7 | P | 461.8 | P |
| | | clone_end = 3/gb = Al849119/gi = 5493025/ug = Mm.1896/len = 507/STRA = rev | | | | | | | |
| 17 | 103285_at | Cluster Incl AF072249.Mus musculus methyl-CpG binding protein MBD4 (Mbd4) mRNA, complete cds/cds = (148,1812)/ | P | 102.6 | P | 133 | P | 152.7 | P |
| | | gb = AF072249/gi = 3800806/ug = Mm 20890/len = 1955/STRA = for | | | | | | | |
| 17 | 103713_at | Cluster Incl AW124533 UI-M-BH2 1-apo-g-12-0-UI s1 Mus musculus cDNA, 3 end/clone = UI-M-BH2 1-apo-g-12-0-UI/ | P | 657.9 | P | 701.5 | P | 733 | P |
| | | clone_end = 3/gb = AW124533/gi = 6100063/ug = Mm 24116/len = 379/STRA = for | | | | | | | |
| 17 | 103780_at | Cluster Incl AW049510 UI-M-BH1-anf-b-05-0-UI s1 Mus musculus cDNA, 3 end/clone = UI-M-BH1-anf-b-05-0-UI/ | P | 215.9 | P | 231.6 | P | 247.9 | P |
| | | clone_end = 3/gb = AW049510/gi = 5910039/ug = Mm 24863/len = 458/STRA = for | | | | | | | |
| 17 | 103781_at | Cluster Incl U76832 Mus musculus plasma membrane protein syntaxin-4 mRNA, complete cds/cds = (0,896)/gb = U76832/ | P | 637.8 | P | 580.2 | P | 664.4 | P |
| | | gi = 1673624/ug = Mm 24867/len = 897/STRA = for | | | | | | | |
| 17 | 103916_at | Cluster Incl AI850713 UI-M-BG1-ail-d-10-0-UI s1 Mus musculus cDNA, 3 end/clone = UI-M-BG1-ail-d-10-0-UI/ | P | 288.1 | P | 351.3 | P | 327.4 | P |
| | | clone_end = 3/gb = AI850713/gi = 5494619/ug = Mm 24984/len = 321/STRA = rev | | | | | | | |
| 17 | 103981_at | Cluster Incl AI842281 UI-M-AM1-afz-e-07-0-UI s1 Mus musculus cDNA, 3 end/clone = UI-M-AM1-afz-e-07-0-UI/ | P | 246 | P | 281 | P | 287.4 | P |
| | | clone_end = 3/gb = AI842281/gi = 5476534/ug = Mm 25837/len = 457/STRA = for | | | | | | | |
| 17 | 104652_at | Cluster Incl AI849601 UI-M-AH1-agr-f-05-0-UI s1 Mus musculus cDNA, 3 end/clone = UI-M-AH1-agr-f-05-0-UI/ | P | 142.9 | P | 155.3 | P | 215.4 | P |
| | | clone_end = 3/gb = AI849601/gi = 5493507/ug = Mm 31570/len = 357/STRA = rev | | | | | | | |
| 17 | 92241_at | Cluster Incl AI849075.UI-M-AH1-agw-e-03-0-UI s1 Mus musculus cDNA, 3 end/clone = UI-M-AH1-agw-e-03-0-UI/ | P | 542.1 | P | 930.8 | P | 837.6 | P |
| | | clone_end = 3/gb = AI849075/gi = 5492981/ug = Mm 34844/len = 355/STRA = rev | | | | | | | |
| 17 | 92350_at | Cluster Incl AA165759 ms60e10 r1 Mus musculus cDNA, 5 end/clone = IMAGE-615978/clone_end = 5/gb = AA165759/ | P | 547.3 | P | 627.9 | P | 600.9 | P |
| | | gi = 1743974/ug = Mm 3926/len = 346/STRA = rev | | | | | | | |
| 17 | 92495_at | Cluster Incl U71269 Mus musculus potential transcriptional repressor No14hp (Not4h) mRNA, complete cds/ | P | 184.6 | P | 255.5 | P | 305.2 | P |
| | | cds = (314,2041)/gb = U71269/gi = 4097901/ug = Mm 42183/len = 2319/STRA = rev | | | | | | | |
| 17 | 92698_at | Cluster Incl U21301 Mus musculus c-mer tyrosine kinase receptor mRNA, complete cds/cds = (49,3033)/gb = U21301/ | P | 138.8 | P | 160.7 | P | 172.7 | P |
| | | gi = 885969/ug = Mm 4582/len = 3564/STRA = for | | | | | | | |
| 17 | 92904_at | Cluster Incl U08185 B lymphocyte induced maturation protein/cds = (220,2790)/gb = U08185/gi = 474283/ug = Mm 4800/ | P | 266.6 | P | 234.1 | P | 367.3 | P |
| | | len = 5281/STRA = rev | | | | | | | |
| 17 | 92986_g_at | Cluster Incl D83203 Mus musculus mRNA, one isoform of PTP-RL9/cds = UNKN0WN/gb = D83203/gi = 4092839/ | P | 392 | P | 461.4 | P | 473.1 | P |
| | | ug = Mm 5046/len = 4208/STRA = for | | | | | | | |
| 17 | 93169_at | Cluster Incl AW212708 uo10g07.x1 Mus musculus cDNA, 3 end/clone = IMAGE-2599164/clone_end = 3/gb = AW212708/ | P | 474.3 | P | 609.7 | P | 580.2 | P |
| | | gi = 6518827/ug = Mm 5343/len = 465/STRA = for | | | | | | | |
| 17 | 93456_r_at | Cluster Incl L47480.Mus musculus BMP-4 gene, complele cds/cds = (92,1354)/gb = L47480/gi = 994733/ug = Mm 6813/ | P | 460.2 | P | 605.6 | P | 404.8 | P |
| | | len = 1654/STRA = for | | | | | | | |
| 17 | 93623_at | Cluster Incl U56724 Mus musculus dystrophin gene, exon 23 and partial cds/cds = (0,212)/gb = U56724/gi = 1336044/ | P | 173.5 | P | 207.1 | P | 183.5 | P |
| | | ug = Mm 742/len = 213/STRA = for | | | | | | | |
| 17 | 93962_at | Cluster Incl AI848598 UI-M-AH1-agv-b-04-0-UI s1 Mus musculus cDNA, 3 end/clone = UI-M-AH1-agv-b-04-0-UI/ | P | 179.7 | P | 249.6 | P | 294.3 | P |
| | | clone_end = 3/gb = AI848598/gi = 5492544/ug = Mm 967/len = 470/STRA = for | | | | | | | |
| 17 | 95287_at | Cluster Incl AA189677 mt70h07 rl Mus musculus cDNA, 5 end/clone = IMAGE-635293/clone_end = 5/gb = AA189677/ | P | 377.8 | P | 361.6 | P | 414 | P |
| | | gi = 1776319/ug = Mm 1873/len = 305/STRA = for | | | | | | | |
| 17 | 95343_at | Cluster Incl AB013848 Mus musculus mRNA for peptidylarginine deiminase type I, complete cds/cds = (127,2115)/ | P | 195.5 | P | 237.1 | P | 260.1 | P |
| | | gb = AB013848/gi = 3845556/ug = Mm 20854/len = 3755/STRA = for | | | | | | | |
| 17 | 95356_at | Cluster Incl D00466 Apolipoprotein E/cds = (23,958)/gb = D00466/gi = 220334/ug = Mm.21417/len = 1068/STRA = for | P | 2691.9 | P | 4302.1 | P | 3960.7 | P |
| 17 | 95368_at | Cluster Incl D86949.Mouse mRNA for plexin 2, complete cds/cds = (579,6233)/gb = D86949/gi = 1655431/ug = Mm 2251/ | P | 279.4 | P | 405 | P | 407.5 | P |
| | | len = 6730/STRA = for | | | | | | | |
| 17 | 96564_at | Cluster Incl X54401 Mouse U14 snRNA (intron 5 of hsc70 gene)/cds = (0,155)/gb = X54401/gi = 55071/ug = Mm 2944/ | P | 3013.5 | P | 4332.5 | P | 4510.3 | P |
| | | len = 156/STRA = for | | | | | | | |
| 17 | 102124_f_at | Cluster Incl AI836879 UI-M-AP0-abg-d-08-0-UI.s2 Mus musculus cDNA, 3 end/clone = UI-M-AP0-abg-d-08-0-UI/ | P | 783.8 | P | 1083.4 | P | 956.9 | P |
| | | clone_end = 3/gb = AI836879/gi = 5471092/ug = Mm 43075/len = 252/STRA = rev | | | | | | | |
| 17 | 102141_f_at | Cluster Incl AI462340 ub72a07 x1 Mus musculus cDNA, 3 end/clone = IMAGE-1383252/clone_end = 3/gb = AI462340/ | P | 367.3 | P | 339 | P | 436.7 | P |
| | | gi = 4316370/ug = Mm 44054/len = 339/STRA = rev | | | | | | | |
| 17 | 102171_r_at | Cluster Incl AI132241 ue32f10 x1 Mus musculus cDNA, 3 end/clone = IMAGE-1482091/clone_end = 3/gb = AI132241/ | P | 795.4 | P | 856.2 | P | 985.8 | P |
| | | gi = 3602257/ug = Mm 46845/len = 735/STRA = rev | | | | | | | |
| 17 | AFFX- | E Coll BioB 5 prime | P | 413.1 | P | 679.3 | P | 628.9 | A |
| | BioB-5_at | | | | | | | | |
| 17 | AFFX-b- | M12481 Mouse cytoplasmic beta-actin mRNA (_5, _M, _3 represent transcript regions 5 prime, | P | 83.2 | P | 141.2 | P | 147.6 | P |
| | ActinMur/ | Middle, and 3 prime respectively) | | | | | | | |
| | M12481_{—} | | | | | | | | |
| | M_st | | | | | | | | |
| 17 | 93040_at | Cluster Incl AF091390 Mus musculus phospholemman precursor, gene, complete cds/cds = (0,278)/gb = AF091390/ | P | 716.7 | P | 689.8 | P | 1095 | P |
| | | gi = 4206710/ug = Mm.1491/len = 279/STRA = for | | | | | | | |
| 17 | 94214_at | Cluster Incl X14961: Fatty acid binding protein heart 1/cds = (21,422)/gb = X14961/gi = 51266/ug = Mm.22220/len = 658/ | P | 176 | P | 200.1 | P | 408.3 | P |
| | | STRA = for | | | | | | | |
| 17 | 96904_at | Cluster Incl AI848784: UI-M-AJ1-ahg-c-10-0-Ul.s1 Mus musculus cDNA, 3 end/clone = UI-M-AJ1-ahg-c-10-0-UI/ | P | 323 | P | 385.7 | P | 378.7 | P |
| | | clone_end = 3/gb = AI848784/gi = 5492690/ug = Mm 30123/len = 490/STRA = for | | | | | | | |
| 17 | 98061_at | Cluster Incl AI841192: UI-M-AM0-ad1-e-02-0-UI s1 Mus musculus cDNA, 3 end/clone = UI-M-AM0-adl-e-02-0-UI/ | P | 370.2 | P | 491 | P | 610.6 | P |
| | | clone_end = 3/gb = AI841192/gi = 5475405/ug = Mm 34386/len = 444/STRA = for | | | | | | | |
| 17 | 99658_f_at | Cluster Incl AW047907: UI-M-BH1-aln-b-05-0-UI s1 Mus musculus cDNA, 3 end/clone = UI-M-BH1-aln-b-05-0-UI/ | P | 587.1 | P | 618.8 | P | 618.5 | P |
| | | clone_end = 3/gb = AW047907/gi = 5908436/ug = Mm 43784/len = 398/STRA = for | | | | | | | |
| 17 | 101546_at | Cluster Incl AF109906 Mus musculus MHC class III region RD gene, partial cds, Bf, C2, G9A, NG22, G9, HSP70, HSP70, | P | 263.4 | P | 255 | P | 335.5 | P |
| | | HSC70t, and smRNP genes, complete cds, G7A gene, partial cds, and unknown genes/cds = (0,1229)/gb = AF109906/ | | | | | | | |
| | | gi = 3986763/ug = Mm 8856/len = 1230/STRA = for | | | | | | | |
| 17 | 103913_at | Cluster Incl AI851315 UI-M-BH0-akf-h-11-0-UI s1 Mus musculus cDNA, 3 end/clone = UI-M-BH0-akf-h-11-0-UI/ | P | 313.8 | P | 341.8 | P | 369.6 | A |
| | | clone_end = 3/gb = AI851315/gi = 5495221/ug = Mm.21435/len = 336/STRA = for | | | | | | | |
| 17 | 104349_at | Cluster Incl AA870301: vq14c07 r1 Mus musculus cDNA, 5 end/clone = IMAGE-1094220/clone_end = 5/gb = AA870301/ | P | 153.3 | P | 227.8 | P | 269.9 | P |
| | | gi = 2965746/ug = Mm.22548/len = 429/STRA = for | | | | | | | |
| 17 | 104356_at | Cluster Incl AI465543 mu92f12.x1 Mus musculus cDNA, 3 end/clone = IMAGE-653039/clone_end = 3/gb = AI465543/ | P | 126.8 | P | 177.2 | P | 186.2 | P |
| | | gi = 4319573/ug = Mm.22581/len = 184/STRA = for | | | | | | | |
| 17 | 94365_at | Cluster Incl AA710175 vt49a11 r1 Mus musculus cDNA, 5 end/clone = IMAGE-1166396/clone_end = 5/gb = AA710175/ | P | 505.6 | P | 610.8 | P | 693.2 | P |
| | | gi = 2720093/ug = Mm.25285/len = 481/STRA = for | | | | | | | |
| 17 | 95633_r_at | Cluster Incl AW122653 UI-M-BH2.2-aol-e-11-0-UI s1 Mus musculus cDNA, 3 end/clone = UI-M-BH2 2-aol-e-11-0-UI/ | P | 371.9 | P | 626.8 | P | 634.1 | P |
| | | clone_end = 3/gb = AW122653/gi = 6098183/ug = Mm 28088/len = 363/STRA = for | | | | | | | |
| 17 | 96732_at | Cluster Incl AI851081 UI-M-BH0-akg-a-12-0-UI s1 Mus musculus cDNA, 3 end/clone = UI-M-BH0-akg-a-12-0-UI/ | P | 506.7 | P | 498.5 | P | 657 | P |
| | | clone_end = 3/gb = AI851081/gi = 5494987/ug = Mm 28891/len = 480/STRA = for | | | | | | | |
| 17 | 99455_s_at | Cluster Incl D17574 Mouse mRNA for proacrosin-binding protein (sp32), complete cds (alternative splicing product)/ | P | 289.9 | P | 281.9 | P | 450.3 | A |
| | | cds = (87,1037)/gb = D17574/gi = 498163/ug = Mm 4073/len = 1253/STRA = for | | | | | | | |
| 17 | 99505_at | Cluster Incl AI844664 UI-M-AL1-ahr-d-11-0-UI.s1 Mus musculus cDNA, 3 end/clone = UI-M-AL1-ahr-d-11-0-UI/ | P | 366.5 | P | 395.2 | P | 415 | P |
| | | clone_end = 3/gb = AI844664/gi = 5488570/ug = Mm 41750/len = 434/STRA = for | | | | | | | |
| 17 | 99527_at | Cluster Incl AB013852 Mus musculus mRNA for Nrf3, complete cds/cds = (237,2219)/gb = AB013852/gi = 4521228/ | P | 66.6 | P | 95.4 | P | 132.4 | P |
| | | ug = Mm 42138/len = 2544/STRA = for | | | | | | | |
| 17 | 100982_at | Cluster Incl AI854539 UI-M-BH0-ajj-e-10-0-UI s1 Mus musculus cDNA, 3 end/clone = UI-M-BHO-ajj-e-10-0-UI/clone_end = 3/ | P | 162 | P | 152.3 | P | 196.4 | P |
| | | gb = AI854539/gi= 5498445/ug = Mm 6720/len = 461/STRA = for | | | | | | | |
| 17 | 101397_at | Cluster Incl AA870756 vq26b08 r1 Mus musculus cDNA, 5 end/clone = IMAGE-1095351/clone_end = 5/gb = AA870756/ | P | 421.2 | P | 479.5 | P | 571.5 | A |
| | | gi = 2966201/ug = Mm 7241/len = 281/STRA = for | | | | | | | |
| 17 | 101919_at | Cluster Incl M32309.Zinc finger protein X-linked/cds = (198,2717)/gb = M32309/gi = 202455/ug = Mm 919/len = 6878/ | A | 299.9 | P | 224.4 | P | 366 | P |
| | | STRA = for | | | | | | | |
| 17 | 102193_at | Cluster Incl AA791891 vs54hO4.r1 Mus musculus cDNA, 5 end/clone = IMAGE-1150135/clone_end = 5/gb = AA791891/ | P | 181 | P | 245 | P | 401.9 | P |
| | | gi = 2854846/ug = Mm 9821/len = 418/STRA = for | | | | | | | |
| 17 | 102196_at | Cluster Incl U37413 Guanine nucleotide binding protein, alpha 11/cds = (57,1136)/gb = U37413/gi = 1051237/ug = Mm 989/ | P | 253.5 | P | 390.9 | P | 319.2 | P |
| | | len = 1342/STRA = rev | | | | | | | |
| 17 | 103315_at | Cluster Incl AW120526 UI-M-BH1-ana-b-09-0-UI s2 Mus musculus cDNA, 3 end/clone = UI-M-BH1-ana-b-09-0-UI/ | A | 221.1 | P | 199.9 | P | 199.3 | P |
| | | clone_end = 3/gb = AW120526/gi = 6095859/ug = Mm 21289/len = 426/STRA = for | | | | | | | |
| 17 | 104007_at | Cluster Incl AA986782 uc74c02 x1 Mus musculus cDNA, 3 end/clone = IMAGE-1431362/clone_end = 3/gb = AA986782/ | P | 295.4 | P | 336.6 | P | 462.7 | P |
| | | gi = 3167784/ug = Mm 26737/len = 394/STRA = rev | | | | | | | |
| 17 | 104398_at | Cluster Incl AI846222 UI-M-AM1-agb-g-06-0-UI s1 Mus musculus cDNA, 3 end/clone = UI-M-AM1-agb-g-06-0-UI/ | P | 135 | P | 185.9 | P | 297.3 | P |
| | | clone_end = 3/gb = AI846222/gi = 5490128/ug = Mm 28655/len = 232/STRA = for | | | | | | | |
| 17 | 92330_r_at | Cluster Incl AA517665 vh81f01 r1 Mus musculus cDNA, 3 end/clone = IMAGE-893401/clone_end = 3/gb = AA517665/ | P | 176.5 | P | 263.8 | P | 340.5 | P |
| | | gi = 2257189/ug = Mm 38891/len = 484/STRA = rev | | | | | | | |
| 17 | 92711_at | Cluster Incl M57625 Mast cell protease 6/cds = UNKNOWN/gb = M57625/gi = 200506/ug = Mm 46216/len = 3757/STRA = for | P | 173.8 | P | 280.5 | P | 306.3 | P |
| 17 | 93130_at | Cluster Incl AW122860 UI-M-BH2.1-aoz-h-09-0-UI s1 Mus musculus cDNA, 3 end/clone = UI-M-BH2 1-aoz-h-09-0-UI/ | P | 201.5 | P | 230.5 | P | 315.2 | P |
| | | clone_end = 3/gb = AW122860/gi = 6098390/ug = Mm 5117/len = 186/STRA = for | | | | | | | |
| 17 | 97706_at | Cluster Incl AA474655 vd53b01.s1 Mus musculus cDNA, 5 end/clone = IMAGE-804265/clone_end = 5/gb = AA474655/ | P | 133.6 | P | 175 | P | 235.3 | P |
| | | gi = 2202810/ug = Mm 38051/len = 305/STRA = for | | | | | | | |
| 17 | 99856_r_at | Cluster Incl AI852919 UI-M-BH0-aiz-b-06-0-UI.s1 Mus musculus cDNA, 3 end/clone = UI-M-BH0-aiz-b-06-0-UI/ | P | 169.4 | P | 269.5 | P | 235.4 | P |
| | | clone_end = 3/gb = AI852919/gi = 5496825/ug = Mm 6680/len = 396/STRA = rev | | | | | | | |
| 17 | 101759_at | Cluster Incl D00851: Mus musculus Tcp-1 pseudogene for I-complex polypeptide 1/cds = (0,1352)/gb = D00851/gi = 220608/ | P | 63.2 | P | 101.3 | P | 88.8 | P |
| | | ug = Mm 57089/len = 1353/STRA = for | | | | | | | |
| 17 | 102583_at | Cluster Incl AJ132195 Mus musculus olfr89 gene, partial/cds = (0,908)/gb = AJ132195/gi = 4186074/ug = Mm 57248/len = 909/ | P | 192.2 | P | 173.5 | P | 294.7 | P |
| | | STRA = for | | | | | | | |
| 17 | 100751_at | Cluster Incl AF011379 A disintegrin and metalloprotease domain (ADAM) 10/cds = (25,2274)/gb = AF011379/gi = 2282607/ | P | 195.3 | P | 206.4 | P | 377.5 | P |
| | | ug = Mm 3911/len = 2481/STRA = for | | | | | | | |
| 17 | 93311_at | Cluster Incl AF033565: Mus musculus cdc2/CDC28-like protein kinase 3 (Clk3) mRNA, complete cds/cds = (63,1535)/ | P | 206.9 | P | 249.9 | P | 212.7 | P |
| | | gb = AF033565/gi = 2645653/ug = Mm 18476/len = 1787/STRA = for | | | | | | | |
| 17 | 97870_s_at | Cluster Incl AA798624 vy03d11.r1 Mus musculus cDNA, 5 end/clone = IMAGE-1294389/clone_end = 5/gb = AA798624/ | P | 349.5 | P | 378.4 | P | 372 | M |
| | | gi = 2861579/ug = Mm 28339/len = 280/STRA = rev | | | | | | | |
| 17 | 98109_at | Cluster Incl AI839526 UI-M-ANO-acn-e-06-0-UI s1 Mus musculus cDNA, 3 end/clone = UI-M-AN0-acn-e-06-0-UI/ | P | 153.8 | P | 144.2 | A | 281.8 | P |
| | | clone_end = 3/gb = AI839528/gi = 5473741/ug = Mm 34801/len = 355/STRA = for | | | | | | | |
| 17 | 99144_s_at | Cluster Incl D50031: Mouse mRNA for TGN38A, complete cds/cds = (26,1087)/gb = D50031/gi = 949828/ug = Mm 4142/ | P | 225.9 | P | 181.1 | A | 481.1 | P |
| | | len = 1673/STRA = for | | | | | | | |
| 17 | 103028_at | Cluster Incl D14042 Mus musculus tlk mRNA for tyrosine kinase, complete cds/cds = (45,1904)/gb = D14042/gi = 220613/ | P | 199.7 | A | 244.5 | M | 294.9 | P |
| | | ug = Mm 16009/len = 4224/STRA = for | | | | | | | |
| 17 | 103057_at | Cluster Incl AF024570: DNA polymerase delta 1, calalytic domain/cds = (0,3317)/gb = AF024570/gi = 2827902/ | P | 349 | P | 367.7 | A | 498.3 | A |
| | | ug = Mm.16549/len = 3318/STRA = rev | | | | | | | |
| 17 | 103914_at | Cluster Incl AI847114: UI-M-AP1-agn-h-04-0-UI s1 Mus musculus cDNA, 3 end/clone = UI-M-AP1-agn-h-04-0-UI/ | P | 536.6 | P | 611.6 | A | 747.5 | P |
| | | clone_end = 3/gb = AI847114/gi = 5491025/ug = Mm.21439/len = 416/STRA = for | | | | | | | |
| 17 | 95604_at | Cluster Incl AI843294: UI-M-AO1-ael-f-03-0-UI s1 Mus musculus cDNA, 3 end/clone = UI-M-AO1-aei-f-03-0-UI/ | P | 540.6 | P | 647.8 | P | 656.5 | P |
| | | clone_end = 3/gb = A1843294/gi = 5477507/ug = Mm 27954/len = 376/STRA = for | | | | | | | |
| 17 | 102698_at | Cluster Incl AF045160 Mus musculus hypoxia-inducible factor 1 alpha related factor mRNA, complete cds/cds = (183,2807)/ | P | 199 | P | 234.2 | P | 247.8 | M |
| | | gb = AF045160/gi = 2895755/ug = Mm.1415/len = 3415/STRA = for | | | | | | | |
| 17 | 103438_at | Cluster Incl AF096875 Mus musculus type 2 deiodinase mRNA, complete cds/cds = (154,954)/gb = AF096875/gi = 4206102/ | M | 158.7 | P | 300.6 | P | 240.7 | P |
| | | ug = Mm 21389/len = 5841/STRA = for | | | | | | | |
| 17 | 104480_at | Cluster Incl AI152659 ud93d03 r1 Mus musculus cDNA, 5 end/clone = IMAGE-1478405/clone_end = 5/gb = AI152659/ | P | 305 | A | 346.6 | P | 364.3 | P |
| | | gi = 3681128/ug = Mm.30027/len = 334/STRA = for | | | | | | | |
| 17 | 92287_at | Cluster Incl AF033187 Mus musculus SOCS-5 mRNA, complete cds/cds = (0,1610)/gb = AF033187/gi = 2766490/ | P | 301.7 | P | 389.4 | A | 484 | P |
| | | ug = Mm.37204/len = 1611/STRA = for | | | | | | | |
| 17 | 92710_at | Cluster Incl U73199 Mus musculus Rho-guanine nucleotide exchange factor mRNA, complete cds/cds = (104,5185)/ | P | 138.2 | P | 124.7 | P | 250.6 | M |
| | | gb = U73199/gi = 1657834/ug = Mm 4620/len = 5405/STRA = for | | | | | | | |
| 17 | 93427_at | Cluster Incl AW122310 UI-M-BH2 2-aos-d-10-0-UI s1 Mus musculus cDNA, 3 end/clone = UI-M-BH2.2-aos-d-10-0-UI/ | P | 214.3 | P | 226.6 | P | 322 | A |
| | | clone_end = 3/gb = AW122310/gi = 6097795/ug = Mm.6493/len = 208/STRA = rev | | | | | | | |
| 17 | 94695_at | Cluster Incl C81411 C81411 Mus musculus cDNA, 3 end/clone = J0098H06/clone_end = 3/gb = C81411/gi = 2521741/ | P | 62.9 | P | 164.3 | A | 265.4 | P |
| | | ug = Mm 13352/len = 586/STRA = rev | | | | | | | |
| 17 | 102584_at | Cluster Incl Y18260 Mus musculus mRNA for protein kinase Dyrk1B/cds = (248,2017)/gb = Y18280/gi = 5738713/ | P | 209.3 | P | 293.2 | P | 299.4 | M |
| | | ug = Mm 57249/len = 2018/STRA = for | | | | | | | |
| 17 | 95782_at | Cluster Incl U19033 Melanoma antigen, related sequence 2/cds = (0,992)/gb = U19033/gi = 1165173/ug = Mm 87790/ | P | 93.6 | P | 170.2 | P | 187.4 | A |
| | | len = 993/STRA = for | | | | | | | |
| 17 | 100713_at | Cluster Incl AB020542: Mus musculus mRNA for Zinc finger protein sf 1-6, complete cds/cds = (106,1869)/gb = AB020542/ | P | 118.6 | P | 145.9 | P | 168.9 | P |
| | | gi = 3953592/ug = Mm.26510/len = 2363/STRA = for | | | | | | | |
| 17 | 92824_at | Cluster Incl AF051942 Mus musculus type 6 nucleoside diphosphate kinase NM23-M6 (Nm23-M6) mRNA, complete cds/ | P | 261.2 | P | 341.9 | P | 307.2 | A |
| | | cds = (121,690)/gb = AF051942/gi = 3228531/ug = Mm 12951/len = 1025/STRA = for | | | | | | | |
| 17 | 97490_at | Cluster Incl AJ011145 Mus musculus mRNA for protein encoded by BCL7B gene/cds = (78,623)/gb = AJ011145/ | P | 126.4 | A | 141.2 | A | 139.4 | P |
| | | gi = 3647321/ug = Mm 31/len = 1546/STRA = for | | | | | | | |
| 17 | 103801_at | Cluster Incl AF094761 Mus musculus Rfxank (Rfxank) mRNA, complete cds/cds = (201,1010)/gb = AF094761/gi = 3820617/ | P | 246.5 | A | 348.7 | P | 360.5 | M |
| | | ug = Mm 20848/len = 1403/STRA = for | | | | | | | |
| 17 | 97433_at | Cluster Incl AJ006590 Mus musculus mRNA for GANP protein/cds = (383,6298)/gb = AJ006590/gi = 4995702/ug = Mm 30098/ | P | 169.6 | P | 257.1 | P | 283.7 | P |
| | | len = 6429/STRA = for | | | | | | | |
| 17 | 103752_r_at | Cluster Incl AW227545 up10e09 x1 Mus musculus cDNA, 3 end/clone = IMAGE-2651656/clone_end = 3/gb = AW227545/ | P | 155.3 | P | 93.9 | P | 359.9 | P |
| | | gi = 6556841/ug = Mm 24516/len = 445/STRA = for | | | | | | | |
| 17 | 104246_at | Cluster Incl AI852340: UI-M-BH0-ajo-b-02-0-UI s1 Mus musculus cDNA, 3 end/clone = UI-M-BH0-ajo-b-02-0-UI/ | A | 367 | A | 497.8 | P | 497.4 | P |
| | | clone_end = 3/gb = AI852340/gi = 5496246/ug = Mm 2833/len = 501/STRA = for | | | | | | | |
| 17 | 104507_g_at | Cluster Incl AF009328 Mus musculus orphan nuclear hormone receptor isoform mCAR2 mRNA, complete cds/ | P | 198.6 | P | 237.5 | A | 279.6 | A |
| | | cds = (244,1104)/gb = AF009328/gi = 2267577/ug = Mm 3077/len = 1332/STRA = for | | | | | | | |
| 17 | 96531_at | Cluster Incl AA153773 mr77h06 r1 Mus musculus cDNA, 5 end/clone = IMAGE-603515/clone_end = 5/gb = AA153773/ | P | 215.4 | P | 297.8 | M | 327 | P |
| | | gi = 1724192/ug = Mm 27295/len = 455/STRA = for | | | | | | | |
| 17 | 96570_at | Cluster Incl AV381276: AV381276 Mus musculus cDNA, 3 end/clone = 9230111G20/clone_end = 3/gb = AV381276/ | A | 53 | P | 47.7 | P | 77.5 | P |
| | | gi = 6430353/ug = Mm 29740/len = 165/STRA = for | | | | | | | |
| 17 | 96600_at | Cluster Incl C76597 C76597 Mus musculus cDNA, 3 end/clone = J0014E03/clone_end = 3/gb = C76597/gi = 2516927/ | P | 50.2 | P | 57.6 | P | 90.7 | P |
| | | ug = Mm 31117/len = 601/STRA = rev | | | | | | | |
| 17 | 99364_at | Cluster Incl AI661370: va11c04 x1 Mus musculus cDNA, 3 end/clone = IMAGE-722598/clone_end = 3/gb = AI661370/ | P | 49.8 | P | 52.5 | P | 58.4 | P |
| | | gi = 4764953/ug = Mm 5633/len = 378/STRA = rev | | | | | | | |
| 17 | 100730_at | Cluster Incl D37797 Lymphocyte antigen 78/cds = (112,2097)/gb = D37797/gi = 761711/ug = Mm 3300/len = 2620/STRA = for | P | 101.4 | P | 131.2 | P | 179.7 | A |
| 17 | 92555_at | Cluster Incl AF053454 Mus musculus tetraspan TM4SF (Tspan-6) mRNA, complete cds/cds = (82,819)/gb = AF053454/ | P | 109.9 | P | 139.4 | A | 195.2 | P |
| | | gi = 2995862/ug = Mm 10180/len = 1058/STRA = for | | | | | | | |
| 17 | 98052_at | Cluster Incl AF003999 Mus musculus Golgi SNARE GS15 mRNA, complete cds/cds = (0,335)/gb = AF003999/gi = 2316079/ | P | 259.3 | P | 272.2 | A | 370.9 | P |
| | | ug = Mm.34315/len = 336/STRA = for | | | | | | | |
| 17 | 101501_r_at | Cluster Incl D87973 Imprinted and ancient/cds = (85,1041)/gb = D87973/gi = 4038075/ug = Mm 8154/len = 3442/STRA = for | P | 101.1 | P | 88.4 | P | 124.2 | P |
| 17 | 94363_at | Cluster Incl AI840815 UI-M-AH0-adb-c-03-0-UI s1 Mus musculus cDNA, 3 end/clone = UI-M-AH0-adb-c-03-0-UI/ | P | 111.9 | P | 115.9 | M | 163 | P |
| | | clone_end = 3/gb = AI840815/gi = 5475028/ug = Mm.25269/len = 363/STRA = for | | | | | | | |
| 17 | 94990_at | Cluster Incl AI854725 UI-M-BHO-akc-g-05-0-UI.s1 Mus musculus cDNA, 3 end/clone = UI-M-BH0-akc-g-05-0-UI/ | A | 160.6 | P | 89.8 | A | 276 | P |
| | | clone_end = 3/gb = AI854725/gi = 5498631/ug = Mm 27312/len = 512/STRA = for | | | | | | | |
| 17 | 96759_r_at | Cluster Incl AW061265: UI-M-BH1-amj-f-10-0-UI s2 Mus musculus cDNA, 3 end/clone = UI-M-BH1-amj-f-10-0-UI/ | P | 363.2 | P | 490.1 | A | 594.7 | A |
| | | clone_end = 3/gb = AW061265/gi = 6009016/ug = Mm 28994/len = 445/STRA = for | | | | | | | |
| 17 | 98965_at | Cluster Incl AC002397: Mouse chromosome 6 BAC-284H12 (Research Genetics mouse BAC library) complete sequence/ | P | 208.9 | P | 352.8 | P | 298.6 | A |
| | | cds = (1,1323)/gb = AC002397/gi = 3287367/ug = Mm 3634/len = 1741/STRA = rev | | | | | | | |
| 17 | 99537_at | Cluster Incl AF100694 Mus musculus Pontin52 mRNA, complete cds/cds = (144,15l4)/gb = AF100694/gi = 4106527/ | A | 260.6 | P | 346.6 | P | 266.8 | A |
| | | ug = Mm 42195/len = 1709/STRA = for | | | | | | | |
| 17 | 101873_at | Cluster Incl X95603: M musculus insl3 gene/cds = (3,368)/gb = X95603/gi = 1771291/ug = Mm 87963/len = 583/STRA = for | M | 93.6 | P | 70.8 | A | 139.8 | P |
| 17 | 103946_at | Cluster Incl U87814 Mus musculus PEST phosphatase Interacting protein mRNA, complete cds/cds = (273,1520)/ | P | 136.8 | P | 133 | A | 160.6 | P |
| | | gb = U87814/gi = 1857711/ug = Mm 2534/len = 1692/STRA = for | | | | | | | |
| 17 | 92246_at | Cluster Incl D85925 Mus musculus mRNA for anagen-specific protein mKAP13, complete cds/cds = (45,548)/gb = D85925/ | P | 131.7 | A | 177.1 | P | 189.8 | P |
| | | gi = 1944386/ug = Mm 3502/len = 844/STRA = for | | | | | | | |
| 17 | 93676_at | Cluster Incl U93583: RAD51 associated protein 1/cds = (43,1056)/gb = U93583/gi = 2697100/ug = Mm.8156/len = 1165/ | A | 322.9 | P | 320.2 | P | 441.5 | P |
| | | STRA = for | | | | | | | |
| 17 | 93933_at | Cluster Incl U89924: Mus musculus protein phosphatase 1 binding protein PTG mRNA, complete cds/cds = (60,944)/ | P | 258 | A | 263.8 | P | 343.1 | P |
| | | gb = U89924/gi = 1888565/ug = Mm 89946/len = 1169/STRA = for | | | | | | | |
| 17 | 95369_at | Cluster Incl AF021345 Mus musculus plasma selenoprotein P (SELP) gene, complete cds/cds = (56,1198)/gb = AF021345/ | A | 78.8 | P | 83.5 | A | 103.7 | P |
| | | gi = 4103141/ug = Mm 22699/len = 2823/STRA = for | | | | | | | |
| 17 | 95955_at | Cluster Incl AI606577 vb90e10 y1 Mus musculus cDNA, 5 end/clone = IMAGE-764298/clone_end = 5/gb = AI606577/ | A | −20.2 | P | 100.3 | P | 122.7 | P |
| | | gi = 4615744/ug = Mm.24932/len = 543/STRA = for | | | | | | | |
| 17 | 98367_at | Cluster Incl X73572 M musculus Hoxd-3 gene, complete CDS/cds = (218,865)/gb = X73572/gi = 313248/ug = Mm 4432/ | A | 66.9 | P | 92.6 | P | 109.1 | P |
| | | len = 2128/STRA = for | | | | | | | |
| 17 | 101157_at | Cluster Incl V00790 Part of messenger RNA for mouse delta-immunoglobulin (codes for part of exon 8 —one of two | P | 279.3 | A | 499.5 | P | 665 | A |
| | | alternate C-termini)/cds = (0,82)/gb = V00790/gi = 51778/ug = Mm.4939/len = 206/STRA = for | | | | | | | |
| 17 | 95670_at | Cluster Incl AI639868: UI-M-AH0-acw-a-05-0-UI s1 Mus musculus cDNA, 3 end/clone = UI-M-AH0-acw-a-05-0-UI/ | A | 127 | P | 109.8 | A | 234 | P |
| | | clone_end = 3/gb = AI839868/gi = 5474081/ug = Mm 29580/len = 406/STRA = for | | | | | | | |
| 17 | 101076_r_at | Cluster Incl AB016592 Mus musculus mRNA for GOB-4, complete cds/cds = (51,578)/gb = AB016592/gi = 3395165/ | P | 73.9 | P | 126.6 | A | 164.7 | A |
| | | ug = Mm.7244/len = 758/STRA = for | | | | | | | |
| 17 | 102352_at | Cluster Incl AW121380.UI-M-BH2 2-aom-f-06-0-UI s1 Mus musculus cDNA, 3 end/clone = UI-M-BH2 2-aom-f-06-0-UI/ | P | 52.9 | A | 82.3 | A | 163.3 | P |
| | | clone_end = 3/gb = AW121380/gi = 6096713/ug = Mm.11360/len = 526/STRA = rev | | | | | | | |
| 17 | 101908_s_at | Cluster Incl AF101164 Mus musculus biliary glycoprotein 2 long isoform (Bgp2) mRNA, complete cds/cds = υ,1120)/ | P | 24.9 | M | 146.8 | P | 209.2 | P |
| | | gb = AF101164/gi = 4092740/ug = Mm 89819/len = 1321/STRA = for | | | | | | | |
| 17 | 102217_at | Cluster Incl AI639925 vr74g08 x1 Mus musculus cDNA, 3 end/clone = IMAGE-1134494/clone_end = 3/gb = AI639925/ | A | 91.8 | P | 125.9 | M | 213.3 | P |
| | | gi = 4703034/ug = Mm 10188/len = 484/STRA = for | | | | | | | |
| 17 | 102240_at | Cluster Incl AF049330: Mus musculus PPAR gamma coactivator (PGC-1) mRNA, complete cds/cds = (91,2484)/ | M | 78.4 | P | 103.1 | P | 115 | M |
| | | gb = AF049330/gi = 3046930/ug = Mm 10707/len = 3029/STRA = for | | | | | | | |
| 17 | 103759_at | Cluster Incl D31788: BP-3 alloantigen/cds = (13,948)/gb = D31788/gi = 559368/ug = Mm.24612/len = 1255/STRA = for | A | 55.6 | P | 49.9 | P | 78.6 | P |
| 17 | 92507_at | Cluster Incl Y12229 Utrophin/cds = (0,10289)/gb = Y12229/gi = 1934962/ug = Mm.42222/len = 11096/STRA = for | M | 118.5 | P | 73.6 | A | 174.4 | P |
| 17 | 93923_at | Cluster Incl AW259620 up31b08 x1 Mus musculus cDNA, 3 end/clone = IMAGE-2655927/clone_end = 3/gb = AW259620/ | A | 653.4 | P | 744.9 | P | 912 | A |
| | | gi = 6632601/ug = Mm.89860/1en = 176/STRA = for | | | | | | | |
| 17 | 94124_at | Cluster Incl AB006074 Mus musculus mTOP3 mRNA for topoisomerase III, complete cds/cds = (265,3276)/gb = AB006074/ | P | 572.5 | P | 663.5 | P | 833.5 | A |
| | | gi = 3061307/ug = Mm 10708/len = 3741/STRA = for | | | | | | | |
| 17 | 95800_s_at | Cluster Incl X53250 Zinc finger protein, autosomal/cds = (0,2228)/gb = X53250/gi = 55468/ug = Mm 87837/len = 2229/ | A | 54.2 | P | 67.8 | A | 64.1 | P |
| | | STRA = for | | | | | | | |
| 17 | 96414_at | Cluster Incl D50656 Mus musculus mRNA for non-selective cation channel, complete cds/cds = (288,1560)/gb = D50656/ | P | 48.4 | A | 118.1 | P | 67.3 | A |
| | | gi = 2924621/ug = Mm.10098/len = 2785/STRA = for | | | | | | | |
| 17 | 102026_s_at | Cluster Incl AB011000 Mus musculus mRNA for choline/ethanolamine kinase, complete cds/cds = (299,1483)/ | P | 328.9 | P | 143.8 | A | 479.7 | P |
| | | gb = AB011000/gi = 2897728/ug = Mm.10124/len = 1708/STRA = for | | | | | | | |
| 17 | 102042_at | Cluster Incl AI842308: UI-M-AM1-afz-a-02-0-Ul.s1 Mus musculus cDNA, 3 end/clone = UI-M-AM1-afz-a-02-0-UI/ | A | 160 | P | 150.8 | P | 156.4 | A |
| | | clone_end = 3/gb = AI842308/gi = 5476486/ug = Mm 10200/len = 475/STRA = for | | | | | | | |
| 17 | 103663_at | Cluster Incl AI837330 UI-M-AK0-adf-d-04-0-UI s1 Mus musculus cDNA, 3 end/clone = UI-M-AK0-adt-d-04-0-UI/ | A | 140.2 | P | 160.2 | P | 200.9 | P |
| | | clone_end = 3/gb = AI837330/gi = 5471543/ug = Mm 2069/len = 332/STRA = for | | | | | | | |
| 17 | 95551_at | Cluster Incl AI849232 UI-M-AJ1-agz-c-07-0-UI.s1 Mus musculus cDNA, 3 end/clone = UI-M-AJ1-agz-c-07-0-UI/ | A | 160.7 | A | 200.2 | P | 303.5 | P |
| | | clone_end = 3/gb = AI849232/gi = 5493138/ug = Mm.27738/len = 419/STRA = for | | | | | | | |
| 17 | 104017_at | Cluster Incl AB033887 Mus musculus mACS4 vanant2 mRNA for Acyl-CoA synthetase 4 vanant2, complete cds/ | P | 44 | A | 60.5 | P | 61.6 | P |
| | | cds = (317,2452)/gb = AB033887/gi = 6172344/ug = Mm 26894/len = 2495/STRA = for | | | | | | | |
| 17 | 92344_at | Cluster Incl AF010600 Mus musculus TNF-response element binding protein mRNA, complete cds/cds = (177,2702)/ | A | 27 | A | 55.6 | P | 63 | P |
| | | gb = AF010600/gi = 2275596/ug = Mm.3909/len = 3064/STRA = for | | | | | | | |
| 17 | 92993_r_at | Cluster Incl AI324972: mm78d11 x1 Mus musculus cDNA, 3 end/clone = IMAGE-534549/clone_end = 3/gb = AI324972/ | P | 24.1 | A | 48.8 | P | 53.6 | P |
| | | gi = 4059401/ug = Mm 5073/len = 431/STRA = rev | | | | | | | |
| 17 | 96539_at | Cluster Incl AW212071 uo81h11.x1 Mus musculus cDNA, 3 end/clone = IMAGE-2648997/clone_end = 3/gb = AW212071/ | A | 65.4 | P | 118.8 | P | 110.1 | P |
| | | gi = 6518072/ug = Mm 27699/len = 402/STRA = for | | | | | | | |
| 17 | 97100_at | Cluster Incl AI854248 UI-M-BH0-ajz-h-11-0-UI.s1 Mus musculus cDNA, 3 end/clone = UI-M-BH0-ajz-h-11-0-UI/ | M | 99.5 | P | 99.1 | P | 181.6 | M |
| | | clone_end = 3/gb = AI854248/gi = 5498154/ug = Mm.32074/len = 516/STRA = rev | | | | | | | |
| 17 | 100739_at | Cluster Incl U96702.Mus musculus serine proteinase inhibitor mBM17 mRNA, partial cds/cds = (0,440)/gb = U96702/ | P | 38.5 | P | 46.6 | A | 80.1 | M |
| | | gi = 2104736/ug = Mm 3518/len = 594/STRA = for | | | | | | | |
| 18 | 92634_at | Cluster Incl U12620: Dipeptidylpeptidase 4/cds = (117,2399)/gb = U12620/gi = 1065904/ug = Mm.1151/len = 4943/STRA = for | P | 253 | P | 244.9 | P | 223 | P |
| 18 | 92643_at | Cluster Incl X74671 Neurofibromatosis 2/cds = (576,2363)/gb = X74671/gi = 456656/ug = Mm.1190/len = 2604/STRA = for | P | 438.6 | P | 462.3 | P | 394.7 | P |
| 18 | 92882_at | Cluster Incl Y00094 Mouse mRNA for ras-related YPT1 protein/cds = (129,746)/gb = Y00094/gi = 55456/ug = Mm 14530/ | P | 683.9 | P | 714.8 | P | 698.3 | P |
| | | len = 1428/STRA = for | | | | | | | |
| 18 | 93090_at | Cluster Incl M23362: Fibroblast growth factor receptor 2/cds = (0,1037)/gb = M23362/gi = 533219/ug = Mm.16340/len = 1038/ | P | 1219.8 | P | 1142.3 | P | 1102 | P |
| | | STRA = for | | | | | | | |
| 18 | 93341_r_at | Cluster Incl AF043120 Mus musculus beta prime coatomer protein mRNA, partial cds/cds = (0,1352)/gb = AF043120/ | P | 1001.4 | P | 1068 | P | 960.1 | P |
| | | gi = 2809536/ug = Mm.18729/1en = 1615/STRA = for | | | | | | | |
| 18 | 93509_at | Cluster Incl U57690: Ubiqultin-conjugating enzyme E2B (RAD6 homology)/cds = (43,501)/gb = U57690/gi = 1373352/ | P | 240 | P | 242.6 | P | 186.8 | P |
| | | ug = Mm 1920/1en = 923/STRA = for | | | | | | | |
| 18 | 93841_at | Cluster Incl AW060249 UI-M-BH1-amv-e-10-0-UI s1 Mus musculus cDNA, 3 end/clone = UI-M-BH1-amv-e-10-0-UI/ | P | 520.7 | P | 546.3 | P | 466.9 | P |
| | | clone_end = 3/gb = AW060249/gi = 6008040/ug = Mm 21617/len = 553/STRA = for | | | | | | | |
| 18 | 94223_at | Cluster Incl AJ010045: Mus musculus mRNA for Rho guanine nucleotide-exchange factor, splice variant NET1A/ | P | 1410.7 | P | 1524.3 | P | 1434.5 | P |
| | | cds = (159,1784)/gb = AJ010045/gi = 4138206/ug = Mm 22261/len = 3290/STRA = for | | | | | | | |
| 18 | 94249_at | Cluster Incl AW226968: um64d03 y1 Mus musculus cDNA, 5 end/clone = IMAGE-2286149/clone_end = 5/gb = AW226968/ | P | 451.2 | P | 500.6 | P | 326 | P |
| | | gi = 6556264/ug = Mm 22374/len = 425/STRA = for | | | | | | | |
| 18 | 94882_at | Cluster Incl AA684506: vr02a10.r1 Mus musculus cDNA, 5 end/clone = IMAGE-1110618/clone_end = 5/gb = AA684506/ | P | 463.8 | P | 313.7 | P | 236.4 | P |
| | | gi = 2670223/ug = Mm.23877/len = 487/STRA = rev | | | | | | | |
| 18 | 95415_f_at | Cluster Incl AI132585 ue97d10 x1 Mus musculus cDNA, 3 end/clone = IMAGE-1499059/clone_end = 3/gb = AI132585/ | P | 453.1 | P | 409.8 | P | 404.9 | P |
| | | gi = 3602592/ug = Mm.24276/len = 597/STRA = for | | | | | | | |
| 18 | 96610_at | Cluster Incl AW046442 UI-M-BH1-akp-e-03-0-UI s1 Mus musculus cDNA, 3 end/clone = UI-M-BH1-akp-e-03-0-UI/ | P | 544.5 | P | 584 | P | 484 | P |
| | | clone_end = 3/gb = AW046442/gi = 5906971/ug = Mm.27082/len = 451/STRA = for | | | | | | | |
| 18 | 96625_at | Cluster Incl AW123132 UI-M-BH2 1-apd-a-04-0-UI s1 Mus musculus cDNA, 3 end/clone = UI-M-BH2 1-apd-a-04-0-UI/ | P | 348.8 | P | 369.3 | P | 288.5 | P |
| | | clone_end = 3/gb = AW123132/gi = 6098882/ug = Mm 2718/len = 488/STRA = for | | | | | | | |
| 18 | 96669_at | Cluster Incl AW122973 UI-M-BH2.1-apb-a-02-0-UI s1 Mus musculus cDNA, 3 end/clone = UI-M-BH2 1-apb-a-02-0-UI/ | P | 468.4 | P | 543.2 | P | 532.5 | P |
| | | clone_end = 3/gb = AW122973/gi = 6098503/ug = Mm 27394/len = 467/STRA = for | | | | | | | |
| 18 | 96302_at | Cluster Incl AA711516 vu26g06 r1 Mus musculus cDNA, 5 end/clone = IMAGE-1181818/clone_end = 5/gb = AA711516/ | P | 469.5 | P | 450.8 | P | 427.5 | P |
| | | gi = 2721434/ug = Mm 29915/len = 260/STRA = rev | | | | | | | |
| 18 | 96879_at | Cluster Incl AI852338 UI-M-BH0-ajo-a-12-0-UI s1 Mus musculus cDNA, 3 end/clone = UI-M-BH0-ajo-a-12-0-UI/ | P | 1317.6 | P | 1547.2 | P | 1054.6 | P |
| | | clone_end = 3/gb = AI852338/gi = 5496244/ug = Mm.30074/len = 475/STRA = for | | | | | | | |
| 18 | 97442_at | Cluster Incl AW124340 UI-M-BH2 1-apq-f-11-0-UI s1 Mus musculus cDNA, 3 end/clone = UI-M-BH2 1-apq-f-11-0-UI/ | P | 557.6 | P | 727.3 | P | 507.6 | P |
| | | clone_end = 3/gb = AW124340/gi = 6099870/ug = Mm 30239/len = 224/STRA = for | | | | | | | |
| 18 | 98055_at | Cluster Incl AW121500 UI-M-BH2.2-aop-a-10-0-UI s1 Mus musculus cDNA, 3 end/clone = UI-M-BH2 2-aop-a-10-0-UI/ | P | 222.5 | P | 298.8 | P | 276.2 | P |
| | | clone_end = 3/gb = AW121500/gi = 6096833/ug = Mm 34330/len = 384/STRA = for | | | | | | | |
| 18 | 98150_at | Cluster Incl L26528 RAB11B, member RAS oncogene family/cds = (35,691)/gb = L26528/gi = 433063/ug = Mm 35727/ | P | 416.4 | P | 387 | P | 326.5 | P |
| | | len = 1763/STRA = for | | | | | | | |
| 18 | 98151_s_at | Cluster Incl Z17804 Catenin src/cds = (342,3077)/gb = Z17804/gi = 53544/ug = Mm 35738/len = 4636/STRA = rev | P | 534.5 | P | 606.4 | P | 586.8 | P |
| 18 | 99119_at | Cluster Incl D00472 Cofilin 1, non-muscle/cds = (94,594)/gb = D00472/gi = 220383/ug = Mm 4024/len = 1134/STRA = for | P | 3003.3 | P | 3848.1 | P | 2968.6 | P |
| 18 | 99639_at | Cluster Incl D84096 Mouse mRNA for ubiquitin carboxyl-terminal hydrolase (UBP), complete cds/cds = (67,2448)/ | P | 251 | P | 375.5 | P | 141.6 | P |
| | | gb = D84096/gi = 1902990/ug = Mm 4355/len = 3236/STRA = for | | | | | | | |
| 18 | 99650_at | Cluster Incl X90945 M. musculus mRNA for casein kinase I-alpha/cds = (0,85)/gb = X90945/gi = 975690/ug = Mm.43737/ | P | 2139.7 | P | 1705.5 | P | 1839.1 | P |
| | | len = 342/STRA = for | | | | | | | |
| 18 | 100069_at | Cluster Incl M77497 Cytochrome P450, 2f2/cds = (61,1536)/gb = M77497/gi = 192927/ug = Mm 4515/len = 1810/STRA = for | P | 419.9 | P | 459.3 | P | 364.3 | P |
| 18 | 100553_at | Cluster Incl L46855 Ret finger protein/cds = (148,1716)/gb = L46855/gi = 1161121/ug = Mm 55/len = 1950/STRA = for | P | 571.5 | P | 710.5 | P | 547 | P |
| 18 | 100616_at | Cluster Incl AF012710.Mus musculus centromere protein A (Cenp-a) gene, promoter region and partial cds/cds = (148,210)/ | P | 351.5 | P | 499.8 | P | 438.1 | P |
| | | gb = AF012710/gi = 2465204/ug = Mm.6579/len = 211/STRA = for | | | | | | | |
| 18 | 101050_at | Cluster Incl AF031381 Mus musculus KOI-4 gene, partial cds/cds = (0,130)/gb = AF031381/gi = 2623677/ug = Mm.7013/ | P | 571.2 | P | 826.6 | P | 516.5 | P |
| | | len = 341/STRA = for | | | | | | | |
| 18 | 101073_at | Cluster Incl X67469 Low density lipoprotein receptor related protein/cds = (441,14078)/gb = X67469/gi = 49941/ug = Mm 7221/ | P | 1162.9 | P | 1126.2 | P | 1148.9 | P |
| | | len = 14849/STRA = for | | | | | | | |
| 18 | 101536_at | Cluster Incl U35312 Mus musculus nuclear receptor co-repressor mRNA, complete cds/cds = (116,7477)/gb = U35312/ | P | 354.7 | P | 357.7 | P | 337.1 | P |
| | | gi = 1022717/ug = Mm 88061/len = 7780/STRA = for | | | | | | | |
| 18 | 101555_at | Cluster Incl X57277 RAS-related C3 botulinum substrate 1/cds = (140,718)/gb = X57277/gi = 53885/ug = Mm 889/len = 886/ | P | 2900.6 | P | 3682.9 | P | 3216.8 | P |
| | | STRA = for | | | | | | | |
| 18 | 102333_at | Cluster Incl AF057286 Mus musculus intersectin-EH binding protein Ibp2 mRNA, partial cds/cds = (0,1530)/gb = AF057286/ | P | 687.3 | P | 717.8 | P | 516.9 | P |
| | | gi = 3063648/ug = Mm 10754/len = 2322/STRA = for | | | | | | | |
| 18 | 102767_at | Cluster Incl AW121700 UI-M-BH2 3-aoc-g-01-0-UI s1 Mus musculus cDNA, 3 end/clone = UI-M-BH2,3-aoc-g-01-0-UI/ | P | 1084.9 | P | 1052.9 | P | 1018.8 | P |
| | | clone_end = 3/gb = AW121700/gi = 6097033/ug = Mm 13080/len = 387/STRA = for | | | | | | | |
| 18 | 103020_s_at | Cluster Incl AI317205 ui35g09 y1 Mus musculus cDNA, 5 end/clone = IMAGE-1853344/clone_end = 5/gb = AI317205/ | P | 498 | P | 792.4 | P | 549.7 | P |
| | | gi = 4032472/ug = Mm 15918/len = 235/STRA = for | | | | | | | |
| 18 | 103029_at | Cluster Incl D86344 Mouse mRNA for Topoisomerase-lnhibitor suppressed, complete cds/cds = (86,1495)/gb = D86344/ | P | 1088.5 | P | 1297.6 | P | 674.9 | P |
| | | gi = 1711237/ug = Mm 1605/len = 2196/STRA = for | | | | | | | |
| 18 | 103073_i_at | Cluster Incl AI842969.UI-M-AO1-aem-g-05-0-UI s1 Mus musculus cDNA, 3 end/clone = UI-M-AO1-aem-g-05-0-UI/ | P | 922.8 | P | 1208.3 | P | 940.4 | P |
| | | clone_end = 3/gb = AI842969/gi = 5477182/ug = Mm 16976/len = 340/STRA = for | | | | | | | |
| 18 | 103099_f_at | Cluster Incl AI663971 ui16h11 r1 Mus musculus cDNA, 5 end/clone = IMAGE-1851525/clone_end = 5/gb = AI663971/ | P | 1324.8 | P | 1575.4 | P | 1365.9 | P |
| | | gi = 4767554/ug = Mm 17537/len = 400/STRA = for | | | | | | | |
| 18 | 103435_at | Cluster Incl AW045910 UI-M-BH1-aku-c-03-0-UI s1 Mus musculus cDNA, 3 end/clone = UI-M-BH1-aku-c-03-0-UI/ | P | 1176.6 | P | 1218.3 | P | 829 | P |
| | | clone_end = 3/gb = AW045910/gi = 5906439/ug = Mm 19073/len = 454/STRA = for | | | | | | | |
| 18 | 103597_at | Cluster Incl X63349 M musculus tyrp2 mRNA for tyrosinase-related protein-2/cds = (404,1957)/gb = X63349/gi = 55065/ | P | 1000.5 | P | 1330.1 | P | 1217.4 | P |
| | | ug = Mm 19987/len = 2182/STRA = for | | | | | | | |
| 18 | 103810_at | Cluster Incl AB010152 Mus musculus mRNA for p73H, complete cds/cds = (118,1878)/gb = AB010152/gi = 3445481/ | P | 863.3 | P | 814.6 | P | 817.6 | P |
| | | ug = Mm 20894/len = 4669/STRA = for | | | | | | | |
| 18 | 103816_at | Cluster Incl U89915: Mus musculus junctional adhesion molecule (Jam) mRNA, complete cds/cds = (70,972)/gb = U89915/ | P | 992.6 | P | 705.1 | P | 848.1 | P |
| | | gi = 3462454/ug = Mm.20903/len = 2029/STRA = for | | | | | | | |
| 18 | 103855_at | Cluster Incl AW123286 UI-M-BH2 1-apg-g-06-0-UI S1 Mus musculus cDNA, 3 end/clone = UI-M-BH2 1-apg-g-06-0-UI/ | P | 463.3 | P | 729.6 | P | 636.8 | P |
| | | clone_end = 3/gb = AW123286/gi = 6098816/ug = Mm 21072/len = 368/STRA = for | | | | | | | |
| 18 | 104625_at | Cluster Incl AA874130 vw87g02 r1 Mus musculus cDNA, 5 end/clone = IMAGE-1261970/clone_end = 5/gb = AA874130/ | P | 585 | P | 689.7 | P | 525.3 | P |
| | | gi = 2978819/ug = Mm 24265/len = 204/STRA = for | | | | | | | |
| 18 | 94375_at | Cluster Incl Y11666 M musculus gene encoding hexokinase II, exon 1 (and joined CDS)/cds = (479,3232)/gb = Y11666/ | P | 340.3 | P | 465.4 | P | 299.6 | P |
| | | gi = 1907082/ug = Mm.2549/len = 5496/STRA = for | | | | | | | |
| 18 | 96185_at | Cluster Incl AB004305 Adaptor-related protein complex AP-3, delta subunit/cds = (220,3819)/gb = AB004305/gi = 2828340 | P | 920.1 | P | 1046.2 | P | 875.2 | P |
| | | ug = Mm 28463/len = 4730/STRA = rev | | | | | | | |
| 18 | 96818_at | Cluster Incl AA762522 vv84d02 r1 Mus musculus cDNA, 5 end/clone = IMAGE-1229091/clone_end = 5/gb = AA762522/ | P | 351.4 | P | 393.3 | P | 371.4 | P |
| | | gi = 2812269/ug = Mm.29343/len = 523/STRA = for | | | | | | | |
| 18 | 97823_at | Cluster Incl AW046532 UI-M-BH1-akz-g-10-0-UI s1 Mus musculus cDNA, 3 end/clone = UI-M-BH1-akz-g-10-0-UI/ | P | 594.9 | P | 503.9 | P | 532.3 | P |
| | | clone/end = 3/gb = AW046532/gi = 5907061/ug = Mm 30167/len = 509/STRA = for | | | | | | | |
| 18 | 97933_at | Cluster Incl AW045317: UI-M-BH1-akn-g-06-0-UI.s1 Mus musculus cDNA, 3 end/clone = UI-M-BH1-akn-g-06-0-UI/ | P | 759.5 | P | 866.4 | P | 675.2 | P |
| | | clone_end = 3/gb = AW045317/gi = 5905846/ug = Mm.30248/len = 324/STRA = for | | | | | | | |
| 18 | 97951_s_at | Cluster Incl U37775 Tuberous sclerosis 2/cds = (34,5259)/gb = U37775/gi = 1181664/ug = Mm 30435/len = 5351/STRA = for | P | 350.9 | P | 426.3 | P | 294.5 | P |
| 18 | 97989_at | Cluster Incl M81483 Mus musculus calmodulin dependent phosphatase catalytic subunit (Cam-Prp) mRNA, 3 end/ | P | 294.3 | P | 290.3 | P | 314.2 | P |
| | | cds = (0,1547)/gb = M81483/gi = 192537/ug = Mm.3150/len = 1800/STRA = rev | | | | | | | |
| 18 | 98033_at | Cluster Incl AA710132: vt45a05 r1 Mus musculus cDNA, 5 end/clone = IMAGE-1166000/clone_end = 5/gb = AA710132/ | P | 1171.9 | P | 1212.4 | P | 973.6 | P |
| | | gi = 2720050/ug = Mm 3311/len = 339/STRA = for | | | | | | | |
| 18 | 98424_at | Cluster Incl D83966 Protein tyrosine phosphatase, non-receptor type 13/cds = (41,7393)/gb = D83966/gi = 1232103/ | P | 576.9 | P | 550.8 | P | 372.5 | P |
| | | ug = Mm 3414/len = 7912/STRA = rev | | | | | | | |
| 18 | 98975_at | Cluster Incl AI019999 ua95g09 r1 Mus musculus cDNA, 5 end/clone = IMAGE-1365280/clone_end = 5/gb = AI019999/ | P | 299.6 | P | 346.1 | P | 339 | P |
| | | gi = 3234335/ug = Mm 36725/len = 486/STRA = for | | | | | | | |
| 18 | 99025_at | Cluster Incl L25125 Mouse RNA helicase and RNA-dependent ATPase from the DEAD box family mRNA, complete cds/ | P | 512.7 | P | 652.6 | P | 515.6 | P |
| | | cds = (97,1533)/gb = L25125/gi = 407991/ug = Mm 3880/len = 2552/STRA = rev | | | | | | | |
| 18 | 99920_at | Cluster Incl U65586: Telomeric repeat binding factor 1/cds = (12,1277)/gb = U65586/gi = 1655898/ug = Mm 4306/len = 1628/ | P | 94.2 | P | 92 | P | 74.7 | P |
| | | STRA = rev | | | | | | | |
| 18 | 100887_at | Cluster Incl AI838562 UI-M-AO0-abz-a-04-0-UI s1 Mus musculus cDNA, 3 end/clone = UI-M-AO0-abz-a-04-0-UI/ | P | 205.1 | P | 298 | P | 322 | P |
| | | clone_end = 3/gb = AI838562/gi = 5472775/ug = Mm 5383/len = 252/STRA = for | | | | | | | |
| 18 | 100889_at | Cluster Incl AI838576 UI-M-AO0-abz-c-02-0-UI s1 Mus musculus cDNA, 3 end/clone = UI-M-AO0-abz-c-02-0-UI/ | P | 419.7 | P | 607 | P | 432.9 | P |
| | | clone_end = 3/gb = AI838576/gi = 5472789/ug = Mm 54120/len = 161/STRA = rev | | | | | | | |
| 18 | 100915_at | Cluster Incl AW125698 UI-M-BH2 2-aqi-f-10-0-UI s1 Mus musculus cDNA, 3 end/clone = UI-M-BH2 2-aqi-f-10-0-UI/ | P | 2218.9 | P | 2914.9 | P | 2216.3 | P |
| | | clone_end = 3/gb = AW125698/gi = 6101228/ug = Mm.5938/len = 128/STRA = for | | | | | | | |
| 18 | 101389_at | Cluster Incl AB008553 Mus musculus mRNA for mLGP85/LIMP II, complete cds/cds = (205,1641)/gb = AB008553/ | P | 333.4 | P | 309.9 | P | 312.4 | P |
| | | gi = 2618485/ug = Mm.7176/len = 1853/STRA = for | | | | | | | |
| 18 | 101851_at | Cluster Incl AF029215 Antigen Identified by monoclonal antibody MRC OX-2/cds = (0,836)/gb = AF029215/gi = 2739125/ | P | 334.8 | P | 421.6 | P | 229.1 | P |
| | | ug = Mm 8649/len = 837/STRA = for | | | | | | | |
| 18 | 102211_r_at | Cluster Incl U66835: Mus musculus unknown protein mRNA, partial cds/cds = (0,98)/gb = U66835/gi = 2970116/ | P | 285.5 | P | 320 | P | 182.9 | P |
| | | ug = Mm 10102/len = 323/STRA = for | | | | | | | |
| 18 | 102670_at | Cluster Incl AI848706 UI-M-AH1-agv-h-09-0-UI s1 Mus musculus cDNA, 3 end/clone = UI-M-AH1-agv-b-09-0-UI/ | P | 432.1 | P | 395.7 | P | 385.6 | P |
| | | clone_end = 3/gb = AI848706/gi = 5492612/ug = Mm.13758/len = 542/STRA = for | | | | | | | |
| 18 | 102930_at | Cluster Incl U14133: Silver/cds = (0,1880)/gb = U14133/gi = 887940/ug = Mm 16756/len = 1881/STRA = for | P | 708.5 | P | 1516.4 | P | 815 | P |
| 18 | 103443_at | Cluster Incl AA711704 vu58c09 r1 Mus musculus cDNA, 5 end/clone = IMAGE-1195600/clone_end = 5/gb = AA711704/ | P | 1785.5 | P | 1872.3 | P | 1845.6 | P |
| | | gi = 2721622/ug = Mm.21476/len = 249/STRA = for | | | | | | | |
| 18 | 103783_at | Cluster Incl AI648965 uk33d12.x1 Mus musculus cDNA, 3 end/clone = lMAGE-1970807/clone_end = 3/gb = AI648965/ | P | 309.4 | P | 287.5 | P | 275.6 | P |
| | | gi = 4729799/ug = Mm.24893/len = 509/STRA = rev | | | | | | | |
| 18 | 104244_at | Cluster Incl AI606891 vw20f11 x1 Mus musculus cDNA, 3 end/clone = IMAGE-1244397/clone_end = 3/gb = AI606891/ | P | 466.1 | P | 400.8 | P | 337.9 | P |
| | | gi = 4616058/ug = Mm 28210/len = 322/STRA = for | | | | | | | |
| 18 | 104248_at | Cluster Incl AW227650 up12b09 y1 Mus musculus cDNA, 5 end/clone = IMAGE-2654105/clone_end = 5/gb = AW227650/ | P | 735.8 | P | 762.1 | P | 779.1 | P |
| | | gi = 6556946/ug = Mm 28345/len = 440/STRA = for | | | | | | | |
| 18 | 104437_at | Cluster Incl Z30174: Zinc finger protein 30/cds = (143,1789)/gb = Z30174/gi = 456268/ug = Mm 2927/len = 2604/STRA = for | P | 430.2 | P | 523.7 | P | 425.6 | P |
| 18 | 92262_at | Cluster Incl AF012923 Mus musculus p53-inducible zinc finger protein (Wig-1) mRNA, complete cds/cds = (173,1045)/ | P | 403.4 | P | 495.6 | P | 200.7 | P |
| | | gb = AF012923/gi = 2804680/ug = Mm 35705/len = 7661/STRA = for | | | | | | | |
| 18 | 92394_f_at | Cluster Incl AB010266 Mus musculus Creb-rp, Tnx and Cyp21 genes for cAMP response element binding protein-related | P | 793.5 | P | 1070.3 | P | 1014.4 | P |
| | | protein, tenascin-X and steroid 21-hydroxylase, partial cds and complete sequences/cds = (0,142)/gb = AB010266/ | | | | | | | |
| | | gi = 2804287/ug = Mm 4068/len = 515/STRA = for | | | | | | | |
| 18 | 92440_at | Cluster Incl U73029 Mus musculus interferon regulatory factor 6 (mirf6) mRNA, complete cds/cds = (141,1544)/gb = U73029/ | P | 810.4 | P | 1016 | P | 931.6 | P |
| | | gi = 1685127/ug = Mm 4179/len = 4046/STRA = rev | | | | | | | |
| 18 | 92915_s_at | Cluster Incl X13961 Mouse rearranged homeotic gene Hox-2 4 (IAP Integration)/cds = (0,731)/gb = X13961/gi = 51393/ | P | 494.6 | P | 640.5 | P | 293.1 | P |
| | | ug = Mm 4822/len = 732/STRA = for | | | | | | | |
| 18 | 92922_at | Cluster Incl U17291: Mus musculus transcription factor AP-2 (AP-2) gene, alternative/cds = (36,602)/gb = U17291/ | P | 903.1 | P | 1183.3 | P | 612.6 | P |
| | | gi = 755199/ug = Mm 4845/len = 603/STRA = for | | | | | | | |
| 18 | 93454_at | Cluster Incl AF081789 Mus musculus cell surface antigen AA4 (AA4) mRNA, complete cds/cds = (120,2054)/gb = AF081789/ | P | 509.6 | P | 447.5 | P | 393.8 | P |
| | | gi = 3695112/ug = Mm 681/len = 3070/STRA = for | | | | | | | |
| 18 | 93875_at | Cluster Incl M12571: Heat shock protein, 70 kDa 3/cds = (0,1262)/gb = M12571/gi = 194014/ug = Mm 88432/len = 1506/ | P | 664.7 | P | 663.3 | P | 630.9 | P |
| | | STRA = rev | | | | | | | |
| 18 | 96506_at | Cluster Incl D83002: Anaplastic lymphoma kinase/cds = (450,5315)/gb = D83002/gi = 1864006/ug = Mm.2536/len = 5767/ | P | 271.7 | P | 364 | P | 255.9 | P |
| | | STRA = for | | | | | | | |
| 18 | 96530_at | Cluster Incl AI503926 vm33f03 x1 Mus musculus cDNA, 3 end/clone = IMAGE-991997/clone_end = 3/gb = AI503926/ | P | 293.2 | P | 277.1 | P | 143.9 | P |
| | | gi = 4401777/ug = Mm 27091/len = 342/STRA = for | | | | | | | |
| 18 | 97722_at | Cluster Incl AA879709 vv97e03 r1 Mus musculus cDNA, 5 end/clone = IMAGE-1230364/clone_end = 5/gb = AA879709/ | P | 519.3 | P | 458 | P | 527.8 | P |
| | | gi = 2988692/ug = Mm.39052/len = 404/STRA = for | | | | | | | |
| 18 | 97738_r_at | Cluster Incl AW121931 UI-M-BH2 3-aoj-e-01-0-UI s1 Mus musculus cDNA, 3 end/clone = UI-M-BH2.3-aoj-e-01-0-UI/ | P | 294.1 | P | 401.8 | P | 393.3 | P |
| | | clone_end = 3/gb = AW121931/gi = 6097377/ug = Mm 3957/len = 367/STRA = rev | | | | | | | |
| 18 | 98308_at | Cluster Incl AJ002522.Mus musculus mRNA for myosin heavy chain 2X, partial/cds = (0,94)/gb = AJ002522/gi = 4582679/ | P | 631.5 | P | 1235.6 | P | 914.4 | P |
| | | ug = Mm 42156/len = 199/STRA = rev | | | | | | | |
| 18 | 100357_g_at | Cluster Incl AW121051 UI-M-BH2 3-aof-d-02-0-UI s1 Mus musculus cDNA, 3 end/clone = UI-M-BH2 3-aof-d-02-0-UI/ | P | 394.1 | P | 488.8 | P | 366.6 | P |
| | | clone_end = 3/gb = AW121051/gi = 6096384/ug = Mm.88761/len = 365/STRA = for | | | | | | | |
| 18 | 101291_at | Cluster Incl Z83956 M musculus spermidine synthase pseudogene/cds = (0,894)/gb = Z83956/gi = 1781038/ug = Mm 56916/ | P | 279.1 | P | 282.4 | P | 281 | P |
| | | len = 895/STRA = for | | | | | | | |
| 18 | 101795_f_at | Cluster Incl U12566 Defensin related cryptdin, related sequence 12/cds = (0,278)/gb = U12566/gi = 531852/ug = Mm 57135/ | P | 802.6 | P | 1050.4 | P | 780.9 | P |
| | | len = 279/STRA = for | | | | | | | |
| 18 | 96375_at | Cluster Incl C80249: C80249 Mus musculus cDNA, 3 end/clone = J0078F09/clone_end = 3/gb = C80249/gi = 2520579/ | P | 204.4 | P | 609.3 | P | 430.4 | P |
| | | ug = Mm.25065/len = 609/STRA = rev | | | | | | | |
| 18 | 102105_f_at | Cluster Incl AI840733: UI-M-AM0-adn-b-04-0-UI s1 Mus musculus cDNA, 3 end/clone = UI-M-AM0-adn-b-04-0-UI/ | P | 292.8 | P | 404.7 | P | 254.3 | P |
| | | clone_end = 3/gb = AI840733/gi = 5474946/ug = Mm 42506/len = 275/STRA = rev | | | | | | | |
| 18 | 96087_at | Cluster Incl L07037 Mannoside acetylglucosaminyl transferase 1/cds = (363,1706)/gb = L07037/gi = 202145/ug = Mm 2672/ | P | 843.7 | P | 500.4 | P | 462.2 | M |
| | | len = 2695/STRA = for | | | | | | | |
| 18 | 95679_at | Cluster Incl AI835912.UI-M-AI0-aao-a-07-0-UI s1 Mus musculus cDNA, 3 end/clone = UI-M-AI0-aao-a-07-0-UI/ | P | 592.2 | P | 657.2 | P | 462.2 | A |
| | | clone_end = 3/gb = AI835912/gi = 5470120/ug = Mm 29609/len = 455/STRA = for | | | | | | | |
| 18 | 97546_at | Cluster Incl AF072127 Mus musculus claudin-1 mRNA, complete cds/cds = (155,790)/gb = AF072127/gi = 3335181/ | P | 142.6 | A | 171.9 | P | 129.1 | P |
| | | ug = Mm 33669/len = 952/STRA = for | | | | | | | |
| 18 | 102750_at | Cluster Incl AF070975 Mus musculus X11 gamma protein mRNA, complete cds/cds = (218,1933)/gb = AF070975/ | P | 338.7 | P | 414.7 | P | 297.7 | A |
| | | gi = 3264793/ug = Mm 12909/len = 2039/STRA = for | | | | | | | |
| 18 | 103629_g_at | Cluster Incl D16503.Lymphoid enhancer binding factor 1/cds = (1804,2658)/gb = D16503/gi = 391762/ug = Mm 2029/ | P | 171.4 | P | 330.9 | P | 69.1 | P |
| | | len = 2740/STRA = rev | | | | | | | |
| 18 | 103900_at | Cluster Incl AW124150 UI-M-BH2 1-apr-f-07-0-UI s1 Mus musculus cDNA, 3 end/clone = UI-M-BH2 1-apr-f-07-0-UI/ | P | 334.2 | P | 294.8 | M | 322.4 | P |
| | | clone_end = 3/gb = AW124150/gi = 6099680/ug = Mm 2133/len = 254/STRA = for | | | | | | | |
| 18 | 95533_at | Cluster Incl AW048037 UI-M-BH1-alv-c-01-0-UI.s1 Mus musculus cDNA, 3 end/clone = UI-M-BH1-alv-c-01-0-UI/ | P | 310.2 | A | 338.3 | P | 376.4 | P |
| | | clone_end = 3/gb = AW048037/gi = 5908531/ug = Mm.27653/len = 396/STRA = for | | | | | | | |
| 18 | 97955_at | Cluster Incl X59513 Mouse 5 end of TRP1 gene for tyrosinase-related protein-1/cds = (185,187)/gb = X59513/gi = 54913/ | P | 115.9 | P | 196.8 | P | 157.9 | A |
| | | ug = Mm 30438/len = 297/STRA = for | | | | | | | |
| 18 | 98490_at | Cluster Incl AA822412: vw37d10r1 Mus musculus cDNA, 5 end/clone = IMAGE-1246003/clone_end = 5/gb = AA822412/ | P | 134 | P | 131.4 | P | 136.4 | A |
| | | gi = 2892280/ug = Mm 35596/len = 460/STRA = for | | | | | | | |
| 18 | 99049_at | Cluster Incl D28492: Caspase 2/cds = (6,1364)/gb = D28492/gi = 3090429/ug = Mm 3921/len = 3395/STRA = for | P | 715.4 | P | 774.9 | P | 667.7 | P |
| 18 | 102230_at | Cluster Incl AF011567: Hyaluronidase 1/cds = (53,1441)/gb = AF011567/gi = 2961469/ug = Mm 10305/len = 2125/STRA = for | P | 163.1 | P | 211.3 | P | 174.5 | P |
| 18 | 95957_at | Cluster Incl C77070 C77070 Mus musculus cDNA, 3 end/clone = J0025F02/clone_end = 3/gb = C77070/gi = 2517400/ | P | 239.8 | P | 280.5 | P | 228.1 | P |
| | | ug = Mm 24934/len = 592/STRA = rev | | | | | | | |
| 18 | 99114_r_at | Cluster Incl AW045905 UI-M-BH1-aku-b-09-0-UI s1 Mus musculus cDNA, 3 end/clone = UI-M-BH1-aku-b-09-0-UI/ | P | 184.8 | P | 268.6 | P | 96.7 | M |
| | | clone_end = 3/gb = AW045905/gi = 5906434/ug = Mm 400/len = 464/STRA = for | | | | | | | |
| 18 | 102787_at | Cluster Incl AI841654 UI-M-AO0-acg-e-05-0-UI s1 Mus musculus cDNA, 3 end/clone = UI-M-AO0-acg-e-05-0-UI/ | P | 384.8 | P | 384.9 | P | 151.7 | A |
| | | clone_end = 3/gb = AI841654/gi = 5475867/ug = Mm 13799/len = 284/STRA = for | | | | | | | |
| 18 | 103668_at | Cluster Incl U40375 Suppressor of Ty 6 (S cerevisiae) homolog/cds = (184,5364)/gb = U40375/gi = 1401056/ug = Mm 20755/ | P | 379.8 | P | 417.6 | P | 278.7 | M |
| | | len = 5923/STRA = for | | | | | | | |
| 18 | 104534_at | Cluster Incl AA623874 vq73h02 s1 Mus musculus cDNA, 5 end/clone = IMAGE-1107987/clone_end = 5/gb = AA623874/ | A | 100.9 | P | 99.4 | P | 94.1 | P |
| | | gi = 2527750/ug = Mm 2325/len = 318/STRA = for | | | | | | | |
| 18 | 99956_at | Cluster Incl Y00864 Mouse c-kit mRNA/cds = (28,2955)/gb = Y00864/gi = 50423/ug = Mm 4394/len = 5098/STRA = for | P | 230.3 | P | 138.4 | P | 133.1 | M |
| 18 | 103281_at | Cluster Incl AF077003: Mus musculus SH3 domain-containing adapter protein mRNA, complete cds/cds = (6,1931)/ | P | 149.6 | P | 215 | P | 118.5 | P |
| | | gb = AF077003/gi = 3550239/ug = Mm 20882/len = 2431/STRA = rev | | | | | | | |
| 18 | 92196_f_at | Cluster Incl X83733 M musculus SAP62-AMH gene/cds = (0,1457)/gb = X83733/gi = 633250/ug = Mm 34039/len = 1458/ | P | 595.6 | P | 1169.2 | P | 694.8 | A |
| | | STRA = for | | | | | | | |
| 18 | 93475_at | Cluster Incl AI845581 UI-M-AQ1-adx-c-02-0-UI s1 Mus musculus cDNA, 3 end/clone = UI-M-AQ1-adx-c-02-0-UI/ | P | 168.4 | P | 193.7 | P | 145.6 | A |
| | | clone_end = 3/gb = AI845581/gi = 5489487/ug = Mm 7086/len = 136/STRA = rev | | | | | | | |
| 18 | 99370_at | Cluster Incl AF095447 Mus musculus natural killer cell receptor NKG2A (Nkg2a) mRNA, complete cds/cds = (0,734)/ | P | 182.8 | M | 184.7 | M | 162.6 | P |
| | | gb = AF095447/gi = 4115906/ug = Mm 56899/len = 735/STRA = for | | | | | | | |
| 18 | 100385_at | Cluster Incl X75832 M musculus Glra1 gene (exon1)/cds = (0,1349)/gb = X75832/gi = 435518/ug = Mm.89320/len = 1350/ | P | 317.1 | P | 370.5 | P | 317.7 | M |
| | | STRA = rev | | | | | | | |
| 18 | 94622_at | Cluster Incl U17252: Mus musculus metabolropic glutamate receptor 8 (mGluR8) mRNA, complete cds/cds = (70,2796)/ | P | 163.9 | P | 249.2 | P | 149.6 | M |
| | | gb = U17252/gi = 854728/ug = Mm 8001/len = 2830/STRA = for | | | | | | | |
| 18 | 99777_s_at | Cluster Incl C76650 C76650 Mus musculus cDNA, 3 end/clone = J0015G08/clone_end = 3/gb = C76650/gi = 2516980/ | P | 89.7 | P | 97 | P | 77.3 | A |
| | | ug = Mm 97081/len = 611/STRA = rev | | | | | | | |
| 18 | 95155_at | Cluster Incl AI853875 UI-M-BH0-ajr-g-11-0-UI.s1 Mus musculus cDNA, 3 end/clone = UI-M-BH0-ajr-g-11-0-UI/ | P | 301.8 | P | 340.4 | A | 388.3 | P |
| | | clone_end = 3/gb = AI853875/gi = 5497781/ug = Mm 29479/len = 416/STRA = for | | | | | | | |
| 18 | 101522_at | Cluster Incl L15429 Mus musculus L6 antigen mRNA, complete cds/cds = (91,699)/gb = L15429/gi = 468909/ug = Mm 856/ | P | 52 | A | 100.2 | A | 76.8 | P |
| | | len = 1077/STRA = for | | | | | | | |
| 18 | 97397_at | Cluster Incl AI848344 UI-M-AH1-ago-d-01-0-UI s1 Mus musculus cDNA, 3 end/clone = UI-M-AH1-ago-d-01-0-UI/ | P | 146.9 | P | 122.2 | P | 109.7 | A |
| | | clone_end = 3/gb = AI848344/gi = 5492250/ug = Mm 29849/len = 404/STRA = for | | | | | | | |
| 18 | 100491_at | Cluster Incl AF045692 Solute carrier family 16 (monocarboxylic acid transporters), member 2/cds = (174,1871)/ | P | 84.8 | P | 122.5 | P | 65.4 | A |
| | | gb = AF045692/gi = 2944355/ug = Mm 5045/len = 4247/STRA = for | | | | | | | |
| 18 | 102228_at | Cluster Incl AF036907: Mus musculus linker for activation of T cells (LAT) mRNA, complete cds/cds = (291,1019)/ | A | 928.4 | P | 813.4 | A | 752.1 | P |
| | | gb = AF036907/gi = 2828027/ug = Mm 10280/len = 1260/STRA = for | | | | | | | |
| 18 | 103303_at | Cluster Incl AA798246: vy05e06.r1 Mus musculus cDNA, 5 end/clone = IMAGE-1294594/clone_end = 5/gb = AA798246/ | P | 94.6 | P | 101.9 | P | 87.4 | P |
| | | gi = 2861201/ug = Mm 21099/len = 494/STRA = for | | | | | | | |
| 18 | 92303_at | Cluster Incl D31898: Protein tyrosine phosphatase, receptor type, R/cds = (355,2325)/gb = D31898/gi = 695408/ug = Mm.3771/ | P | 664.6 | P | 929.3 | P | 429.7 | M |
| | | len = 3430/STRA = for | | | | | | | |
| 18 | 94197_at | Cluster Incl D89866: Mus musculus mRNA for ceramide glucosyltransferase, complete cds/cds = (185,1369)/gb = D89866/ | P | 82 | P | 30.2 | P | 46.7 | A |
| | | gi = 3219161/ug = Mm.12971/len = 1523/STRA = for | | | | | | | |
| 18 | 94733_at | Cluster Incl J03398 P glycoprotein 2/cds = (193,4023)/gb = J03398/gi = 199109/ug = Mm 14172/len = 4084/STRA = for | P | 271.3 | P | 425.4 | P | 338.5 | P |
| 18 | 98846_f_at | Cluster Incl M83344 Carcinoembryonic antigen 2/cds = (192,1619)/gb = M83344/gi = 200316/ug = Mm 5056/len = 2043/ | P | 479.3 | P | 638.8 | P | 365.8 | A |
| | | STRA = for | | | | | | | |
| 18 | 101229_at | Cluster Incl C78228: C78228 Mus musculus cDNA, 3 end/clone = J0044F11/clone_end = 3/gb = C78228/gi = 2518558/ | P | 79.5 | P | 145.1 | P | 185.4 | A |
| | | ug = Mm 5692/len = 553/STRA = rev | | | | | | | |
| 18 | 93516_at | Cluster Incl M25825: T-complex lestis expressed 1/cds = (12,353)/gb = M25825/gi = 201912/ug = Mm.1948/len = 680/ | A | 75.9 | P | 150.2 | P | 102.2 | A |
| | | STRA = for | | | | | | | |
| 18 | 94211_at | Cluster Incl AA959180 ua07g06 r1 Mus musculus cDNA, 5 end/clone = IMAGE-1346074/clone_end = 5/gb = AA959180/ | A | 185.3 | P | 311.9 | P | 203.6 | A |
| | | gi = 3124373/ug = Mm 22214/len = 347/STRA = for | | | | | | | |
| 18 | 97239_at | Cluster Incl AI845106: UI-M-BG0-ahs-a-04-0-UI s1 Mus musculus cDNA, 3 end/clone = UI-M-BG0-ahs-a-04-0-UI/ | P | 43.3 | A | 72.2 | A | 54.3 | A |
| | | clone_end = 3/gb = AI845106/gi = 5489012/ug = Mm 27839/len = 141/STRA = for | | | | | | | |
| 18 | 103055_r_at | Cluster Incl M12130 RNA polymerase II 1/cds = (0,5798)/gb = M12130/gi = 200793/ug = Mm 16533/len = 5799/STRA = rev | P | 809.2 | A | 948.2 | P | 756.1 | A |
| 18 | 104079_at | Cluster Incl AW124678 UI-M-BH2 1-apw-g-03-0-UI s1 Mus musculus cDNA, 3 end/clone = UI-M-BH2 1-apw-g-03-0-UI/ | A | 180.6 | P | 91.7 | P | −45.5 | A |
| | | clone_end = 3/gb = AW124678/gi = 6100208/ug = Mm.21735/len = 406/STRA = for | | | | | | | |
| 18 | 95011_at | Cluster Incl AI853090 UI-M-BH0-ajf-b-06-0-UI s1 Mus musculus cDNA, 3 end/clone = UI-M-BH0-ajf-b-06-0-UI/ | P | 141.4 | P | 203 | P | 52.6 | A |
| | | clone_end = 3/gb = AI853090/gi = 5496996/ug = Mm 27434/len = 444/STRA = for | | | | | | | |
| 18 | 95606_at | Cluster Incl AF093821 Mus musculus RRM RNA binding protein GRY-RBP mRNA, complete cds/cds = (6,1883)/ | A | 58 | P | 44.9 | A | 29.2 | M |
| | | gb = AF093821/gi = 3694985/ug = Mm 27972/len = 2373/STRA = for | | | | | | | |
| 18 | 97368_at | Cluster Incl U95145 Mus musculus S-AKAP84 mRNA, complete cds/cds = (197,1840)/gb = U95145/gi = 2072304/ | P | 922.9 | P | 1050 | A | 631.4 | A |
| | | ug = Mm 2969/len = 2965/STRA = for | | | | | | | |
| 18 | 97372_at | Cluster Incl AA880432: vw89f04.r1 Mus musculus cDNA, 5 end/clone = IMAGE-1262143/clone_end = 5/gb = AA880432/ | P | 354.1 | P | 511.1 | P | 87.7 | A |
| | | gi = 2989415/ug = Mm 29701/len = 200/STRA = for | | | | | | | |
| 18 | 99523_at | Cluster Incl AA684502 vr02a04 r1 Mus musculus cDNA, 5 end/clone = IMAGE-1110606/clone_end = 5/gb = AA684502/ | P | 482.2 | P | 610.6 | A | 618.2 | A |
| | | gi = 2670219/ug = Mm 4206/len = 479/STRA = for | | | | | | | |
| 18 | 99931_at | Cluster Incl U37501 Laminin, alpha 5/cds = (0,10907)/gb = U37501/gi = 2599231/ug = Mm 4339/len = 11009/STRA = rev | A | 215.6 | P | 202.4 | P | 46.2 | P |
| 18 | 100508_at | Cluster Incl AF015769 Mus musculus manic fringe (manic-fringe) mRNA, complete cds/cds = (0,965)/gb = AF015769/ | P | 290.4 | A | 329.1 | A | 260.4 | A |
| | | gi = 2454571/ug = Mm 517/len = 966/STRA = for | | | | | | | |
| 18 | 101932_at | Cluster Incl D83484 Mouse mRNA for protein tyrosine phosphatase epsilon, complete cds/cds = (660,2759)/gb = D83484/ | A | 150.4 | P | 190.4 | A | 204.7 | P |
| | | gi = 1199932/ug = Mm 945/len = 2827/STRA = for | | | | | | | |
| 18 | 92754_at | Cluster Incl D49920 Mouse mRNA for ferredoxin-NADP reductase, complete cds/cds = (82,1566)/gb = D49920/gi = 1088468/ | P | 258 | M | 338.6 | P | 202.6 | M |
| | | ug = Mm 4719/len = 1866/STRA = rev | | | | | | | |
| 18 | 92936_at | Cluster Incl X14943 Contactin 1/cds = (249,3311)/gb = X14943/gi = 50937/ug = Mm 4911/len = 3843/STRA = for | P | 156.9 | P | 191.1 | P | 81.3 | A |
| 18 | 98312_at | Cluster Incl AB016768 Mus musculus mRNA for thrombospondin type 1 domain, complete cds/cds = (482,1279)/ | A | 178.6 | P | 233.5 | M | 69.4 | A |
| | | gb = AB016768/gi = 4519540/ug = Mm 42202/len = 1834/STRA = for | | | | | | | |
| 18 | 98385_at | Cluster Incl D31842: Mouse mRNA for putative protein tyrosine phosphatase (PTP36), complete cds/cds = (133,3702)/ | P | 222.9 | P | 289.1 | P | 239 | A |
| | | gb = D31842/gi = 507330/ug = Mm 4498/len = 3957/STRA = rev | | | | | | | |
| 18 | 101148_at | Cluster Incl D28577 Protein kinase C, lamda/cds = (36,1796)/gb = D28577/gi = 3452685/ug = Mm 4801/len = 1966/STRA = for | P | 133.2 | A | 215.3 | P | 147.2 | P |
| 18 | 97894_at | Cluster Incl AF109905 Mus musculus major histocompatibility locus class III regions Hsc70t gene, partial cds, smRNP, | P | 306.1 | P | 422.3 | A | 243.9 | A |
| | | G7A, NG23, Muts homolog, CLCP, NG24, NG25, and NG26 genes, complete cds, and unknown genes/cds = (0,3791)/ | | | | | | | |
| | | gb = AF109905/gi = 3986751/ug = Mm 2/STRA = for | | | | | | | |
| 18 | 94463_at | Cluster Incl X78874: Chloride channel 3/cds = (375,2657)/gb = X78874/gi = 854275/ug = Mm.28842/len = 2758/STRA = for | P | 54.2 | P | 71.4 | P | 52.6 | A |
| 18 | 102769_f_at | Cluster Incl AB016248 Mus musculus mRNA for sterol-C5-desaturase, complete cds/cds = (170,1069)/gb = AB016248/ | P | 31.4 | A | 57.7 | A | 33.4 | P |
| | | gi = 3721883/ug = Mm 13081/len = 1721/STRA = for | | | | | | | |
| 18 | 103635_at | Cluster Incl AI648925 uk32h09 x1 Mus musculus cDNA, 3 end/clone = IMAGE-1970753/clone_end = 3/gb = AI648925/ | P | 122 | P | 152.5 | P | 80.9 | A |
| | | gi = 4729759/ug = Mm 20320/len = 594/STRA = for | | | | | | | |
| 18 | 104326_at | Cluster Incl AI838951 UI-M-AO0-ace-e-06-0-UI s1 Mus musculus cDNA, 3 end/clone = UI-M-AO0-ace-e-06-0-UI/ | P | 335 | P | 314.4 | A | 228 | A |
| | | clone_end = 3/gb = AI838951/gi = 5473164/ug = Mm 22456/len = 451/STRA = for | | | | | | | |
| 18 | 96817_at | Cluster Incl AI850550 UI-M-BG1-aij-d-11-0-UI s1 Mus musculus cDNA, 3 end/clone = UI-M-BG1-aij-d-11-0-UI/ | P | 238.1 | P | 305.7 | A | 234.8 | A |
| | | clone_end = 3/gb = AI850550/gi = 5494456/ug = Mm 29333/len = 282/STRA = for | | | | | | | |
| 18 | 100906_at | Cluster Incl M68903 Integrin beta 7/cds = (39,2456)/gb = M68903/gi = 349598/ug = Mm 58/len = 2636/STRA = rev | P | 342.1 | P | 408.3 | M | 383.9 | A |
| 18 | 103756_at | Cluster Incl AA832886 uc91h02 r1 Mus musculus cDNA, 5 end/clone = IMAGE-1433043/clone_end = 5/gb = AA832886/ | P | 237 | P | 285 | P | 55.4 | A |
| | | gi = 2906614/ug = Mm.24569/len = 395/STRA = rev | | | | | | | |
| 18 | 93391_at | Cluster Incl AF032995: Crystallin, gamma S/cds = (51,587)/gb = AF032995/gi = 2642336/ug = Mm 6253/len = 702/STRA = for | A | 77.6 | P | 81.2 | P | 29.7 | A |
| 18 | 93902_at | Cluster Incl AJ250669: Mus musculus gab1 gene for GRB2-associated binding protein 1/cds = (23,2110)/gb = AJ250669/ | P | 80.9 | M | 58.2 | A | 38.6 | P |
| | | gi = 6165414/ug = Mm.89347/len = 2620/STRA = for | | | | | | | |
| 18 | 94111_r_at | Cluster Incl D10627: Mus musculus mRNA for zinc finger protein, partial cds/cds = (0,1040)/gb = D10627/gi = 220636/ | P | 80.5 | P | 136.3 | P | 88.5 | A |
| | | ug = Mm 10509/len = 1041/STRA = for | | | | | | | |
| 18 | 96502_at | Cluster Incl U75646 Phosphate regulating neutral endopeptidases on the X Chromosome/cds = (111,2360)/gb = U75646/ | M | 113 | P | 110 | P | 71.3 | A |
| | | gi = 1843530/ug = Mm.2529/len = 2636/STRA = for | | | | | | | |
| 18 | 97107_at | Cluster Incl AA162144 mr19b05 r1 Mus musculus cDNA, 5 end/clone = IMAGE-597873/clone_end = 5/gb = AA162144/ | A | 79.5 | P | 101.3 | P | 70.6 | P |
| | | gi = 1737885/ug = Mm.3261/len = 300/STRA = rev | | | | | | | |
| 18 | 99894_at | Cluster Incl AF006201: Mus musculus prostaglandin F2 alpha receptor regulatory protein mRNA, partial cds/cds = (0,198)/ | A | 179.6 | A | 340.7 | P | 230.1 | A |
| | | gb = AF006201/gi = 2653775/ug = Mm 7999/len = 271/STRA = for | | | | | | | |
| 18 | 100701_r_at | Cluster Incl AB000490 Mouse mRNA for ELP3, complete cds/cds = (245,1636)/gb = AB000490/gi = 1805352/ug = Mm 1416/ | P | 162.8 | P | 180.4 | M | 139.5 | A |
| | | len = 2981/STRA = for | | | | | | | |
| 18 | 101705_at | Cluster Incl X12809: Mouse period repetitive mRNA (lambda Sp34)/cds = (30,212)/gb = X12809/gi = 53648/ug = Mm 41987/ | A | 229.7 | P | 321.8 | A | 151.7 | A |
| | | len = 522/STRA = for | | | | | | | |
| 18 | 93778_at | Cluster Incl AA717815 vt98f07 r1 Mus musculus cDNA, 5 end/clone = IMAGE-1179205/clone_end = 5/gb = AA717815/ | P | 422.4 | P | 693.3 | A | 481.1 | A |
| | | gi = 2730089/ug = Mm 21239/len = 424/STRA = for | | | | | | | |
| 18 | 104608_at | Cluster Incl AI854293: UI-M-BH0-ajs-e-11-0-UI s1 Mus musculus cDNA, 3 end/clone = UI-M-BH0-ajs-e-11-0-UI/ | A | 77.1 | P | 105.7 | A | 63.1 | A |
| | | clone_end = 3/gb = AI854293/gi = 5498199/ug = Mm 24143/len = 511/STRA = for | | | | | | | |
| 18 | 94349_at | Cluster Incl AA794923: vr48g02.s1 Mus musculus cDNA, 5 end/clone = IMAGE-1123922/clone_end = 5/gb = AA794923/ | M | 119 | A | 136.7 | M | 80.7 | A |
| | | gi = 2857878/ug = Mm 25189/len = 537/STRA = for | | | | | | | |
| 18 | 97980_at | Cluster Incl L38423: Lymphotoxin B receptor/cds = (185,1432)/gb = L38423/gi = 600222/ug = Mm.3122/len = 1614/STRA = for | P | 313 | P | 328.3 | P | 207.1 | A |
| 18 | 101857_at | Cluster Incl AB006036 Serine/arginine-rich protein specific kinase 2/cds = (0,2045)/gb = AB006036/gi = 2723281/ | P | 87.3 | P | 99.8 | M | 84.1 | A |
| | | ug = Mm B709/len = 2300/STRA = for | | | | | | | |
| 18 | 103473_at | Cluster Incl AA623401 vn32c09.r1 Mus musculus cDNA, 5 end/clone = IMAGE-1022896/clone_end = 5/gb = AA623401/ | P | 277.5 | A | 329.9 | P | 354.7 | M |
| | | gi = 2527277/ug = Mm 21876/len = 345/STRA = for | | | | | | | |
| 18 | 104413_at | Cluster Incl AI834976 UI-M-AM1-afw-d-12-0-UI s1 Mus musculus cDNA, 3 end/clone = UI-M-AM1-afw-d-12-0-UI/ | P | 108 | P | 117.5 | A | 22.8 | M |
| | | clone_end = 3/gb = AI834976/gi = 5469189/ug = Mm.28885/len = 434/STRA = for | | | | | | | |
| 18 | 93899_at | Cluster Incl AJ000654 Mouse p70/p85 S6 kinase gene, 5 flanking region and the first exon/cds = (63,203)/gb = AJ000654/ | A | 79.4 | P | 125.5 | P | 123.7 | A |
| | | gi = 2330619/ug = Mm 89315/len = 204/STRA = for | | | | | | | |
| 18 | 100326_f_at | Cluster Incl U12889 Killer cell lectin-like receptor, subfamily A, member B/cds = (196,996)/gb = U12889/gi = 602407/ | A | 46 | P | 42.5 | M | 45 | A |
| | | ug = Mm.87782/len = 1229/STRA = for | | | | | | | |
| 18 | 96961_at | Cluster Incl AI503821: vm20c01.x1 Mus musculus cDNA, 3 end/clone = IMAGE-990720/clone_end = 3/gb = AI503821/ | A | 102.1 | P | 189.3 | A | 125 | A |
| | | gi = 4401672/ug = Mm 69094/len = 470/STRA = rev | | | | | | | |
| 18 | 102131_f_at | Cluster Incl AU014874 AU014874 Mus musculus cDNA, 3 end/clone = J0703H02/clone_end = 3/gb = AU014874/ | P | 450 | A | 552 | A | 381.3 | A |
| | | gi = 3369665/ug = Mm 43527/len = 294/STRA = rev | | | | | | | |
| 19 | 92616_at | Cluster Incl D10576 Ubiquitin-activating enzyme E1, Chr X/cds = (91,3267)/gb = D10576/gi = 220628/ug = Mm 1104/ | P | 1191.4 | P | 1449.7 | P | 1453.2 | P |
| | | len = 3458/STRA = for | | | | | | | |
| 19 | 92793_at | Cluster Incl X57796 Tumor necrosis factor receptor superfamily, member 1a/cds = (272,1636)/gb = X57796/gi = 54848/ | P | 672.9 | P | 474.6 | P | 520.5 | P |
| | | ug = Mm 1258/len = 2179/STRA = for | | | | | | | |
| 19 | 93047_at | Cluster Incl AI849144 UI-M-AJ1-agy-c-07-0-UI.s1 Mus musculus cDNA, 3 end/clone = UI-M-AJ1-agy-c-07-0-UI/ | P | 736.2 | P | 912 | P | 863.7 | P |
| | | clone_end = 3/gb = AI849144/gi = 5493050/ug = Mm.1521/len = 439/STRA = for | | | | | | | |
| 19 | 93083_at | Cluster Incl D63423: Mouse mRNA for annexin V, complete cds/cds = (81,1040)/gb = D63423/gi = 939830/ug = Mm 1620/ | P | 1147.2 | P | 1227.8 | P | 1624.1 | P |
| | | len = 1415/STRA = for | | | | | | | |
| 19 | 93255_at | Cluster lncl X80937 Ral-interacting protein 1/cds = (202,2148)/gb = X80937/gi = 984748/ug = Mm 17009/len = 3661/ | P | 1335.8 | P | 1400.7 | P | 1702.1 | P |
| | | STRA = rev | | | | | | | |
| 19 | 93268_at | Cluster Incl AI852001 UI-M-BH0-aiq-f-01-0-UI s1 Mus musculus cDNA, 3 end/clone = UI-M-BH0-aiq-f-01-0-UI/ | P | 567 | P | 551.1 | P | 708.7 | P |
| | | clone_end = 3/gb = AI852001/gi = 5495907/ug = Mm 17362/len = 431/STRA = for | | | | | | | |
| 19 | 93269_at | Cluster Incl AI848952 UI-M-AJ1-ahd-a-06-0-UI s1 Mus musculus cDNA, 3 end/clone = UI-M-AJ1-ahd-a-06-0-UI/ | P | 885.9 | P | 1467.9 | P | 1201.6 | P |
| | | clone_end = 3/gb = AI848952/gi = 5492858/ug = Mm 17362/len = 482/STRA = for | | | | | | | |
| 19 | 93315_at | Cluster Incl AI852636 UI-M-BH0-aiu-g-03-0-Ul.s1 Mus musculus cDNA, 3 end/clone = UI-M-BH0-aiu-g-03-0-UI/ | P | 663.3 | P | 729.8 | P | 721.9 | P |
| | | clone_end = 3/gb = AI852636/gi = 5496542/ug = Mm 18494/len = 365/STRA = for | | | | | | | |
| 19 | 93323_at | Cluster Incl AB031292 Mus musculus Plp2 mRNA for proteolipid protein 2, complete cds/cds = (58,516)/gb = AB031292/ | P | 3510.8 | P | 3638.5 | P | 3811.6 | P |
| | | gi = 5771450/ug = Mm 18585/len = 603/STRA = for | | | | | | | |
| 19 | 93336_at | Cluster Incl AW121539: UI-M-BH2 2-aop-e-01-0-UI s1 Mus musculus cDNA, 3 end/clone = UI-M-BH2 2-aop-e-01-0-UI/ | P | 1271.3 | P | 1165 | P | 1419.9 | P |
| | | clone_end = 3/gb = AW121539/gi = 6096872/ug = Mm 18688/len = 514/STRA = for | | | | | | | |
| 19 | 93753_at | Cluster Incl AI852632: UI-M-BH0-aiu-f-10-0-UI.s1 Mus musculus cDNA, 3 end/clone = UI-M-BH0-aiu-f-10-0-UI/ | P | 704.8 | P | 456.8 | P | 672.6 | P |
| | | clone_end = 3/gb = AI852632/gi = 5496538/ug = Mm 21119/len = 346/STRA = for | | | | | | | |
| 19 | 93809_at | Cluster Incl U41736 Ancient ubiquitous protein/cds = (60,1292)/gb = U41736/gi = 1517821/ug = Mm.2146/len = 1499/ | P | 794.4 | P | 952.3 | P | 897 | P |
| | | STRA = for | | | | | | | |
| 19 | 93843_at | Cluster Incl AI845580 UI-M-AQ1-adx-c-01-0-UI s1 Mus musculus cDNA, 3 end/clone = UI-M-AQ1-adx-c-01-0-UI/ | P | 738.6 | P | 699.5 | P | 1111.5 | P |
| | | clone_end = 3/gb = AI845580/gi = 5489486/ug = Mm.21623/len = 245/STRA = for | | | | | | | |
| 19 | 94297_at | Cluster Incl U16959 FK506 binding protein 5, 51 kDa/cds = (219,1589)/gb = U16959/gi = 915279/ug = Mm 22595/len = 2285/ | P | 329.8 | P | 182.6 | P | 715 | P |
| | | STRA = for | | | | | | | |
| 19 | 94805_f_at | Cluster Incl M33988 Mouse histone H2A 1 gene, complete cds/cds = (45,437)/gb = M33988/gi = 193730/ug = Mm 22676/ | P | 2689.6 | P | 2892.7 | P | 4269.8 | P |
| | | len = 506/STRA = for | | | | | | | |
| 19 | 94813_at | Cluster Incl X65128 Growth arrest specific 1/cds = (424,1578)/gb = X65128/gi = 51045/ug = Mm 22701/len = 2961/STRA = for | P | 1466.5 | P | 1369.9 | P | 2055 | P |
| 19 | 94850_at | Cluster Incl AJ238894 Mus musculus mRNA for mitochondrial acyl-CoA thioesterase, clone 2/cds = (97,1416)/ | P | 622.7 | P | 528.9 | P | 694.1 | P |
| | | gb = AJ238894/gi = 5102775/ug = Mm 23368/len = 1557/STRA = for | | | | | | | |
| 19 | 94868_at | Cluster Incl AW049812 UI-M-BH1-anm-h-04-0-UI s1 Mus musculus cDNA, 3 end/clone = UI-M-BH1-anm-h-04-0-UI/ | P | 1028.9 | P | 941.2 | P | 1549.8 | P |
| | | clone_end = 3/gb = AW049812/gi = 5910341/ug = Mm 23750/len = 571/STRA = for | | | | | | | |
| 19 | 94881_at | Cluster Incl AW048937 UI-M-BH1-amo-d-08-0-UI s1 Mus musculus cDNA, 3 end/clone = UI-M-BH1-amo-d-08-0-UI/ | P | 1036.9 | P | 1071.6 | P | 1294.2 | P |
| | | clone_end = 3/gb = AW048937/gi = 5909466/ug = Mm 2387/len = 355/STRA = for | | | | | | | |
| 19 | 95446_at | Cluster Incl AW120502 UI-M-BH1-ami-f-05-0-UI s2 Mus musculus cDNA, 3 end/clone = UI-M-BH1-ami-f-05-0-UI/ | P | 1099.5 | P | 962.9 | P | 1186.3 | P |
| | | clone_end = 3/gb = AW120502/gi = 6095835/ug = Mm 24600/len = 199/STRA = for | | | | | | | |
| 19 | 95520_at | Cluster Incl AI843845 UI-M-AK1-aeu-a-10-0-UI s1 Mus musculus cDNA, 3 end/clone = UI-M-AK1-aeu-a-10-0-UI/ | P | 242 | P | 307.8 | P | 269.4 | P |
| | | clone_end = 3/gb = AI843845/gi = 5478058/ug = Mm 25320/len = 404/STRA = for | | | | | | | |
| 19 | 96056_at | Cluster Incl X80638 M. musculus rhoC mRNA/cds = (40,621)/gb = X80638/gi = 1279555/ug = Mm.262/len = 1036/STRA = for | P | 653.9 | P | 738 | P | 864.8 | P |
| 19 | 96060_at | Cluster Incl U25844 Serine protease inhibitor 3/cds = (67,1203)/gb = U25844/gi = 818902/ug = Mm 2623/len = 1317/ | P | 1072 | P | 883.2 | P | 1600.1 | P |
| | | STRA = for | | | | | | | |
| 19 | 96102_i_at | Cluster Incl X92411 RAD23b homolog (S cerevisiae)/cds = (0,1250)/gb = X92411/gi = 1044898/ug = Mm 26879/len = 1251/ | P | 500.2 | P | 534 | P | 568.3 | P |
| | | STRA = for | | | | | | | |
| 19 | 96113_at | Cluster Incl AI846519 UI-M-AN1-aff-e-06-0-UI s1 Mus musculus cDNA, 3 end/clone = UI-M-AN1-aff-e-06-0-UI/ | P | 768.3 | P | 874.7 | P | 678 | P |
| | | clone_end = 3/gb = AI846519/gi = 5490425/ug = Mm 26956/len = 234/STRA = for | | | | | | | |
| 19 | 96117_r_at | Cluster Incl AI843732: UI-M-AK1-aer-g-09-0-UI s1 Mus musculus cDNA, 3 end/clone = UI-M-AK1-aer-g-09-0-UI/ | P | 527.5 | P | 347.6 | P | 519 | P |
| | | clone_end = 3/gb = AI843732/gi = 5477945/ug = Mm 26991/len = 369/STRA = for | | | | | | | |
| 19 | 96709_at | Cluster Incl AI839839: UI-M-AP0-abf-a-09-0-UI s2 Mus musculus cDNA, 3 end/clone = UI-M-AP0-abf-a-09-0-UI/ | P | 769.4 | P | 687.1 | P | 675.1 | P |
| | | clone_end = 3/gb = AI839839/gi = 5474052/ug = Mm 27608/len = 281/STRA = for | | | | | | | |
| 19 | 97885_at | Clusler Incl AB031386 Mus musculus mRNA for Clast1, complete cds/cds = (196,987)/gb = AB031386/gi = 5804793/ | P | 1022 | P | 1127.3 | P | 1323.1 | P |
| | | ug = Mm 28385/len = 1207/STRA = for | | | | | | | |
| 19 | 98884_r_at | Cluster Incl AI837311: UI-M-AK0-adf-b-03-0-UI.s1 Mus musculus cDNA, 3 end/clone = UI-M-AK0-adf-b-03-0-UI/ | P | 952.1 | P | 875.5 | P | 990.9 | P |
| | | clone_end = 3/gb = AI837311/gi = 5471524/ug = Mm.28527/len = 420/STRA = for | | | | | | | |
| 19 | 94465_g_at | Cluster Incl AF029347: Mus musculus chloride channel protein 3 (CLCN3) mRNA, complete cds/cds = (0,2456)/ | P | 136.5 | P | 164.8 | P | 176.1 | P |
| | | gb = AF029347/gi = 2599549/ug = Mm.28842/len = 2457/STRA = for | | | | | | | |
| 19 | 94473_at | Cluster Incl AW047951: UI-M-BH1-aln-f-04-0-UI s1 Mus musculus cDNA, 3 end/clone = UI-M-BH1-aln-f-04-0-UI/ | P | 651.2 | P | 368.5 | P | 704.8 | P |
| | | clone_end = 3/gb = AW047951/gi = 5908480/ug = Mm 28853/len = 408/STRA = for | | | | | | | |
| 19 | 94484_at | Cluster Incl AW227778: up14b01.y1 Mus musculus cDNA, 5 end/clone = IMAGE-2654281/clone_end = 5/gb = AW227778/ | P | 691 | P | 696.4 | P | 566.5 | P |
| | | gi = 6557074/ug = Mm 28882/len = 460/STRA = for | | | | | | | |
| 19 | 94485_at | Cluster Incl AI840013 UI-M-AH0-acu-e-04-0-UI s1 Mus musculus cDNA, 3 end/clone = UI-M-AH0-acu-e-04-0-UI/ | P | 950.8 | P | 951.4 | P | 1019.4 | P |
| | | clone_end = 3/gb = AI840013/gi = 5474226/ug = Mm 28883/len = 356/STRA = for | | | | | | | |
| 19 | 94530_at | Cluster Incl AI840376 UI-M-AH0-acv-d-02-0-UI s1 Mus musculus cDNA, 3 end/clone = UI-M-AH0-acv-d-02-0-UI/ | P | 230.9 | P | 178.3 | P | 344.8 | P |
| | | clone_end = 3/gb = AI840376/gi = 5474589/ug = Mm 29041/len = 448/STRA = for | | | | | | | |
| 19 | 95082_at | Cluster Incl AI842277 UI-M-AM1-afz-e-02-0-UI.s1 Mus musculus cDNA, 3 end/clone = UI-M-AM1-afz-e-02-0-UI/ | P | 789.9 | P | 351.9 | P | 1181.4 | P |
| | | clone_end = 3/gb = AI842277/gi = 5476530/ug = Mm.29254/len = 475/STRA = for | | | | | | | |
| 19 | 95129_at | Cluster Incl AW121057 UI-M-BH2 3-aof-d-08-0-UI s1 Mus musculus cDNA, 3 end/clone = UI-M-BH2 3-aof-d-08-0-UI/ | P | 944.7 | P | 985.1 | P | 1039.2 | P |
| | | clone_end = 3/gb = AW121057/gi = 6096390/ug = Mm 29412/len = 220/STRA = rev | | | | | | | |
| 19 | 95133_at | Cluster Incl U38940 Mus musculus asparagine synthetase mRNA, complete cds/cds = (98,1783)/gb = U38940/gi = 1147604/ | P | 1042.2 | P | 1249 | P | 1389.5 | P |
| | | ug = Mm.2942/len = 1928/STRA = for | | | | | | | |
| 19 | 95721_at | Cluster Incl AW120722 UI-M-BH2.3-any-a-09-0-UI s1 Mus musculus cDNA, 3 end/clone = UI-M-BH2 3-any-a-09-0-UI/ | P | 1131.9 | P | 1044.1 | P | 1086.2 | P |
| | | clone_end = 3/gb = AW120722/gi = 6096055/ug = Mm 29725/len = 406/STRA = for | | | | | | | |
| 19 | 96278_at | Cluster Incl AI846553 UI-M-AN1-aff-h-09-0-UI s1 Mus musculus cDNA, 3 end/clone = UI-M-AN1-aff-h-09-0-UI/ | P | 325.4 | P | 258 | P | 267 | P |
| | | clone_end = 3/gb = AI846553/gi = 5490459/ug = Mm 29865/len = 300/STRA = for | | | | | | | |
| 19 | 96359_at | Cluster Incl AI836554 UI-M-AP0-abe-e-12-0-UI s1 Mus musculus cDNA, 3 end/clone = UI-M-AP0-abe-e-12-0-UI/ | P | 1185.7 | P | 1329.6 | P | 1224.1 | P |
| | | clone_end = 3/gb = AI836554/gi = 5470767/ug = Mm 30012/len = 514/STRA = for | | | | | | | |
| 19 | 96919_at | Cluster Incl M64298 ATPase-like vacuolar proton channel/cds = (147,614)/gb = M64298/gi = 199901/ug = Mm.30155/ | P | 2535.6 | P | 3009.2 | P | 3457.1 | P |
| | | len = 1153/STRA = for | | | | | | | |
| 19 | 96935_at | Cluster Incl AW011791 ul91f11 x1 Mus musculus cDNA, 3 end/clone = IMAGE-2159469/clone_end = 3/gb = AW011791/ | P | 699.7 | P | 768.4 | P | 801.6 | P |
| | | gi = 5860569/ug = Mm 30181/len = 290/STRA = for | | | | | | | |
| 19 | 96953_at | Cluster Incl AW120786 UI-M-BH2 3-any-g-10-0-UI s1 Mus musculus cDNA, 3 end/clone = UI-M-BH2 3-any-g-10-0-UI/ | P | 698.6 | P | 736.6 | P | 801.5 | P |
| | | clone_end = 3/gb = AW120786/gi = 6096119/ug = Mm 30211/len = 401/STRA = for | | | | | | | |
| 19 | 97487_at | Cluster Incl X70296 Serine protease Inhibitor 4/cds = (148,1341)/gb = X70296/gi = 551064/ug = Mm 3093/len = 2024/ | P | 188.4 | P | 208.3 | P | 179.3 | P |
| | | STRA = for | | | | | | | |
| 19 | 97498_at | Cluster Incl U41739: Mus musculus RBP associated molecule RAM14-1 mRNA, complete cds/cds = (82,924)/gb = U41739/ | P | 2702 | P | 2444.4 | P | 4242.8 | P |
| | | gi = 2894671/ug = Mm.3126/len = 2270/STRA = for | | | | | | | |
| 19 | 97500_g_at | Cluster Incl AI839950 UI-M-AH0-acs-a-10-0-UI s1 Mus musculus cDNA, 3 end/clone = UI-M-AH0-acs-a-10-0-UI/ | P | 1598.5 | P | 1303.4 | P | 2575.5 | P |
| | | clone_end = 3/gb = AI839950/gi = 5474163/ug = Mm 3126/len = 361/STRA = for | | | | | | | |
| 19 | 97551_at | Cluster Incl AI846182 UI-M-AM1-agb-c-09-0-UI.s1 Mus musculus cDNA, 3 end/clone = UI-M-AM1-agb-c-09-0-UI/ | P | 489.4 | P | 385.9 | P | 635.4 | P |
| | | clone_end = 3/gb = AI846182/gi = 5490088/ug = Mm 33797/len = 312/STRA = for | | | | | | | |
| 19 | 98051_at | Cluster Incl AI852808 UI-M-BH0-aly-g-09-0-UI.s1 Mus musculus cDNA, 3 end/clone = UI-M-BH0-aly-g-09-0-UI/ | P | 547.3 | P | 607.9 | P | 689.4 | P |
| | | clone_end = 3/gb = AI852808/gi = 5496714/ug = Mm 34315/len = 359/STRA = for | | | | | | | |
| 19 | 98057_at | Cluster Incl AW121162 UI-M-BH2 3-aoh-g-10-0-UI s1 Mus musculus cDNA, 3 end/clone = UI-M-BH2 3-aoh-g-10-0-UI/ | P | 158.2 | P | 124.8 | P | 184.6 | P |
| | | clone_end = 3/gb = AW121162/gi = 6096495/ug = Mm 34356/len = 429/STRA = for | | | | | | | |
| 19 | 98081_at | Cluster Incl AI853173: UI-M-BH0-ajh-b-07-0-UI s1 Mus musculus cDNA, 3 end/clone = UI-M-BH0-ajh-b-07-0-UI/ | P | 332.3 | P | 221.2 | P | 229.7 | P |
| | | clone_end = 3/gb = AI853173/gi = 5497079/ug = Mm 34570/len = 366/STRA = for | | | | | | | |
| 19 | 98560_at | Cluster Incl AI849152: UI-M-AJ1-agy-d-04-0-UI s1 Mus musculus cDNA, 3 end/clone = UI-M-AJ1-agy-d-04-0-UI/ | P | 520 | P | 526.8 | P | 687.5 | P |
| | | clone_end = 3/gb = AI849152/gi = 5493058/ug = Mm 36879/len = 435/STRA = for | | | | | | | |
| 19 | 98580_at | Cluster Incl D28117 Mouse mRNA for magnesium dependent protein phosphatase (protein phosphatase 2C) alpha, | P | 580.3 | P | 515.5 | P | 678.9 | P |
| | | complete cds/cds = (117,1265)/gb = D28117/gi = 532678/ug = Mm 3766/len = 1387/STRA = for | | | | | | | |
| 19 | 99099_at | Cluster Incl U08378 Mus musculus acute phase response factor (APRF) mRNA, complete cds/cds = (146,2455)/ | P | 855.6 | P | 636.5 | P | 763.6 | P |
| | | gb = U08378/gi = 473889/ug = Mm 3948/len = 2924/STRA = for | | | | | | | |
| 19 | 99606_at | Cluster Incl AB005132: LIM motif-containing protein kinase 2/cds = (355,2208)/gb = AB005132/gi = 2804552/ug = Mm 42927/ | P | 499.8 | P | 630.7 | P | 686.4 | P |
| | | len = 3688/STRA = for | | | | | | | |
| 19 | 99622_at | Cluster Incl U20344 Gut enriched Kruppel-like factor/cds = (310,1761)/gb = U20344/gi = 912487/ug = Mm 4325/len = 2756/ | P | 2694.7 | P | 1833.7 | P | 3021 | P |
| | | STRA = for | | | | | | | |
| 19 | 99635_at | Cluster Incl AI845183 UI-M-BG0-aht-a-12-0-UI s1 Mus musculus cDNA, 3 end/clone = UI-M-BG0-aht-a-12-0-UI/ | P | 598.6 | P | 956.1 | P | 591.9 | P |
| | | clone_end = 3/gb = AI845183/gi = 5489089/ug = Mm 43482/len = 412/STRA = for | | | | | | | |
| 19 | 99636_at | Cluster Incl AI848033: UI-M-AH1-agp-f-05-0-UI s1 Mus musculus cDNA, 3 end/clone = UI-M-AH1-agp-f-05-0-UI/ | P | 443.5 | P | 407.1 | P | 505.9 | P |
| | | clone_end = 3/gb = AI848033/gi = 5491939/ug = Mm 43487/len = 257/STRA = for | | | | | | | |
| 19 | 99638_at | Cluster Incl D17546 Procoliagen, type XV/cds = (0,3422)/gb = D17546/gi = 2160436/ug = Mm 4352/len = 4437/STRA = for | P | 557.2 | P | 682 | P | 586.6 | P |
| 19 | 100095_at | Cluster Incl U37799 Scavenger receptor class B1/cds = (50,1579)/gb = U37799/gi = 1167551/ug = Mm 4603/len = 1785/ | P | 689.8 | P | 715.3 | P | 838.2 | P |
| | | STRA = for | | | | | | | |
| 19 | 100134_at | Cluster Incl X77952 Endoglin/cds = (363,2324)/gb = X77952/gi = 458473/ug = Mm 4851/len = 2902/STRA = for | P | 460.2 | P | 520.5 | P | 534.9 | P |
| 19 | 100530_at | Cluster Incl L07924 Mus musculus guanine nucleotide dissociation stimulator for a ras-related GTPase mRNA, complete | P | 824.1 | P | 625.9 | P | 1036.9 | P |
| | | cds/cds = (212,2770)/gb = L07924/gi = 193572/ug = Mm 5236/len = 3573/STRA = for | | | | | | | |
| 19 | 100573_f_at | Cluster Incl M14220 Mouse neuroleukin mRNA, complete cds/cds = (52,1728)/gb = M14220/gi = 200064/ug = Mm 589/ | P | 440.4 | P | 484.6 | P | 760.3 | P |
| | | len = 1985/STRA = for | | | | | | | |
| 19 | 100593_at | Cluster Incl L47600 Mus musculus cardiac troponin T isoform A3b mRNA, complete cds/cds = (56,922)/gb = L47600/ | P | 138.8 | P | 164.8 | P | 220.9 | P |
| | | gi = 1161079/ug = Mm 632/len = 1111/STRA = for | | | | | | | |
| 19 | 100599_at | Cluster Incl M94087: Activating transcription factor 4/cds = (270,1310)/gb = M94087/gi = 293723/ug = Mm 641/len = 1391/ | P | 1404 | P | 1370.4 | P | 1177.5 | P |
| | | STRA = for | | | | | | | |
| 19 | 100636_at | Cluster Incl U28656: Eukaryotic translation Initiation factor 4E binding protein 1/cds = (50,403)/gb = U28656/gi = 881557/ | P | 414.1 | P | 427 | P | 495.2 | P |
| | | ug = Mm.6700/len = 793/STRA = for | | | | | | | |
| 19 | 101011_at | Cluster Incl L25913: Chaperonin subunit 4 (delta)/cds = (0,1619)/gb = L25913/gi = 460316/ug = Mm.6821/len = 1847/ | P | 1760.2 | P | 1820.3 | P | 1975.6 | P |
| | | STRA = for | | | | | | | |
| 19 | 101089_at | Cluster Incl AF002283 Mus musculus alpha-actinin-2 associated LIM protein mRNA, alternatively spliced product, complete | P | 594 | P | 570.7 | P | 949.4 | P |
| | | cds/cds = (119,1069)/gb = AF002283/gi = 3138925/ug = Mm 7342/len = 1485/STRA = for | | | | | | | |
| 19 | 101591_at | Cluster Incl AI852589 UI-M-BH0-alu-b-07-0-UI s1 Mus musculus cDNA, 3 end/clone = UI-M-BH0-alu-b-07-0-UI/ | P | 412.6 | P | 366.5 | P | 493 | P |
| | | clone_end = 3/gb = AI852589/gi = 5496495/ug = Mm.91334/len = 235/STRA = for | | | | | | | |
| 19 | 101975_at | Cluster Incl Z12171 DIk1-like homolog (Drosophila)/cds = (0,1157)/gb = Z12171/gi = 50716/ug = Mm 95633/len = 1158/ | P | 3552.6 | P | 4258.5 | P | 5076.1 | P |
| | | STRA = for | | | | | | | |
| 19 | 102072_g_at | Cluster Incl AI851565 UI-M-BH0-aki-f-01-0-UI.s1 Mus musculus cDNA, 3 end/clone = UI-M-BH0-aki-f-01-0-UI/clone_end = 3/ | P | 377.3 | P | 401.1 | P | 533.7 | P |
| | | gb = AI851565/gi = 5495471/ug = Mm 10641/len = 341/STRA = for | | | | | | | |
| 19 | 102332_at | Cluster Incl AI850194 UI-M-BG1-aie-a-01-0-UI s1 Mus musculus cDNA, 3 end/clone = UI-M-BG1-aie-a-01-0-UI/ | P | 292.5 | P | 270.4 | P | 316.1 | P |
| | | clone_end = 3/gb = AI850194/gi = 5494100/ug = Mm 10750/len = 294/STRA = rev | | | | | | | |
| 19 | 102389_s_at | Cluster Incl AI841303: UI-M-AM0-adu-h-03-0-UI s1 Mus musculus cDNA, 3 end/clone = UI-M-AM0-adu-h-03-0-UI/ | P | 177.6 | P | 77 | P | 222.2 | P |
| | | clone_end = 3/gb = AI841303/gi = 5475516/ug = Mm 1222/len = 472/STRA = for | | | | | | | |
| 19 | 102807_at | Cluster Incl AW048054 UI-M-BH1-alv-g-07-0-UI s1 Mus musculus cDNA, 3 end/clone = UI-M-BH1-alv-g-07-0-UI/ | P | 286.2 | P | 326.3 | P | 338.4 | P |
| | | clone_end = 3/gb = AW048054/gi = 5908583/ug = Mm 14155/len = 457/STRA = for | | | | | | | |
| 19 | 102815_at | Cluster Incl U65986: Annexin XI/cds = (134,1645)/gb = U65986/gi = 1815638/ug = Mm 1427/len = 2370/STRA = for | P | 1055.1 | P | 963.7 | P | 1419.1 | P |
| 19 | 102835_at | Cluster Incl X14972 Mouse mRNA for alpha-adaptin (C)/cds = (150,2966)/gb = X14972/gi = 49879/ug = Mm 14555/len = 3242/ | P | 410 .4 | P | 330.7 | P | 428.2 | P |
| | | STRA = for | | | | | | | |
| 19 | 103002_at | Cluster Incl M27923 Glycoprotein galactosyltransferase beta 1, 4/cds = (216,1376)/gb = M27923/gi = 609527/ug = Mm.15622/ | P | 1202 | P | 1214.6 | P | 1619.2 | P |
| | | len = 1540/STRA = for | | | | | | | |
| 19 | 103559_at | Cluster Incl M12303 Protein kinase, cAMP dependent, catalytic, alpha/cds = (186,1241)/gb = M12303/gi = 200366/ | P | 650.6 | P | 917.6 | P | 930.4 | P |
| | | ug = Mm 19111/len = 2231/STRA = for | | | | | | | |
| 19 | 103584_at | Cluster Incl AW124334 UI-M-BH2.1-apq-f-05-0-UI s1 Mus musculus cDNA, 3 end/clone = UI-M-BH2 1-apq-f-05-0-UI/ | P | 830.7 | P | 994.5 | P | 1027.8 | P |
| | | clone_end = 3/gb = AW124334/gi = 6099864/ug = Mm 19802/len = 227/STRA = rev | | | | | | | |
| 19 | 104041_at | Cluster Incl AW122255 UI-M-BH2 2-aov-g-06-0-UI s1 Mus musculus cDNA, 3 end/clone = UI-M-BH2.2-aov-g-06-0-UI/ | P | 915.3 | P | 901.8 | P | 1067.1 | P |
| | | clone_end = 3/gb = AW122255/gi = 6097722/ug = Mm 21458/len = 454/STRA = for | | | | | | | |
| 19 | 104058_at | Cluster Incl AW047528 UI-M-BH1-ama-d-09-0-UI s1 Mus musculus cDNA, 3 end/clone = UI-M-BH1-ama-d-09-0-UI/ | P | 787 | P | 718.5 | P | 873.2 | P |
| | | clone_end = 3/gb = AW047528/gi = 5908057/ug = Mm 21537/len = 418/STRA = for | | | | | | | |
| 19 | 104083_at | Cluster Incl AI853217 UI-M-BH0-ajh-f-11-0-UI s1 Mus musculus cDNA, 3 end/clone = UI-M-BH0-ajh-f-11-0-UI/ | P | 487.5 | P | 335.9 | P | 383.1 | P |
| | | clone_end = 3/gb = AI853217/gi = 5497123/ug = Mm 21767/len = 230/STRA = for | | | | | | | |
| 19 | 104110_at | Cluster Incl AW060515: UI-M-BH1-ann-d-07-0-UI s1 Mus musculus cDNA, 3 end/clone = UI-M-BH1-ann-d-07-0-UI/ | P | 934.6 | P | 825.7 | P | 1015.8 | P |
| | | clone_end = 3/gb = AW060515/gi = 6008266/ug = Mm 21919/len = 330/STRA = for | | | | | | | |
| 19 | 104121_at | Cluster Incl M90365 Junction plakoglobin/cds = (0,1865)/gb = M90365/gi = 1389681/ug = Mm 21990/len = 1878/STRA = rev | P | 2185.7 | P | 2120.8 | P | 2363.9 | P |
| 19 | 104139_at | Cluster Incl U16162 Mus musculus prolyl 4-hydroxylase alpha(l)-subunit mRNA, partial cds/cds = (0,1582)/gb = U16162/ | P | 164.8 | P | 224 | P | 210.5 | P |
| | | gi = 836897/ug = Mm 2212/len = 2129/STRA = for | | | | | | | |
| 19 | 104145_at | Cluster Incl U81030 Treacher Collins Franceschetti syndrome 1, homolog/cds = (48,3956)/gb = U81030/gi = 2145148/ | P | 439.5 | P | 592.4 | P | 581.9 | P |
| | | ug = Mm 2215/len = 4428/STRA = for | | | | | | | |
| 19 | 104554_at | Cluster Incl L25674: Mus musculus ear-2 transcription factor mRNA, complete cds/cds = (113,1282)/gb = L25674/gi = 409757/ | P | 442 | P | 502.7 | P | 634.3 | P |
| | | ug = Mm 2376/len = 1643/STRA = for | | | | | | | |
| 19 | 104714_at | Cluster Incl AW125299 UI-M-BH2 1-apy-c-05-0-UI s1 Mus musculus cDNA, 3 end/clone = UI-M-BH2 1-apy-c-05-0-UI/ | P | 376.7 | P | 465.8 | P | 526.3 | P |
| | | clone_end = 3/gb = AW125299/gi = 6100829/ug = Mm 24477/len = 374/STRA = for | | | | | | | |
| 19 | 94345_at | Cluster Incl AI843709: UI-M-AK1-aer-e-08-0-UI s1 Mus musculus cDNA, 3 end/clone = UI-M-AK1-aer-e-08-0-UI/ | P | 430.5 | P | 322.4 | P | 405.6 | P |
| | | clone_end = 3/gb = AI843709/gi = 5477922/ug = Mm 25142/len = 419/STRA = rev | | | | | | | |
| 19 | 94357_at | Cluster Incl AA591002 vn61e07.r1 Mus musculus cDNA, 5 end/clone = IMAGE-1025700/clone_end = 5/gb = AA591002/ | P | 54.5 | P | 94.2 | P | 111.5 | P |
| | | gi = 2404315/ug = Mm.25237/len = 504/STRA = for | | | | | | | |
| 19 | 95016_at | Cluster Incl D50086 Mouse mRNA for neuropilin, complete cds/cds = (347,3118)/gb = D50086/gi = 1783300/ug = Mm.27448/ | P | 893.6 | P | 1082.1 | P | 1283.3 | P |
| | | len = 3652/STRA for | | | | | | | |
| 19 | 95023_at | Cluster Incl AW050353 UI-M-BH1-ang-d-12-0-UI.s1 Mus musculus cDNA, 3 end/clone = UI-M-BH1-ang-d-12-0-UI/ | P | 1525.2 | P | 1064.8 | P | 1722.2 | P |
| | | clone_end = 3/gb = AW050353/gi = 5910882/ug = Mm 27490/len = 514/STRA = for | | | | | | | |
| 19 | 95602_at | Cluster Incl AI848135 UI-M-AP1-agk-h-05-0-UI s1 Mus musculus cDNA, 3 end/clone = UI-M-AP1-agk-h-05-0-UI/ | P | 1179.1 | P | 1141.3 | P | 1546.4 | P |
| | | clone_end = 3/gb = AI848135/gi = 5492041/ug = Mm 27942/len = 365/STRA = for | | | | | | | |
| 19 | 96157_at | Cluster Incl AI839059 UI-M-AK0-adi-c-06-0-UI s1 Mus musculus cDNA, 3 end/clone = UI-M-AK0-adi-c-06-0-UI/ | P | 1064.5 | P | 1156.6 | P | 1095.7 | P |
| | | clone_end = 3/gb = AI839059/gi = 5473272/ug = Mm 28324/len = 514/STRA = for | | | | | | | |
| 19 | 96748_i_at | Cluster Incl AA619554 vo88e08 r1 Mus musculus cDNA, 5 end/clone = IMAGE-1066214/clone_end = 5/gb = AA619554/ | P | 145.3 | P | 164.6 | P | 159 | P |
| | | gi = 2523430/ug = Mm 28958/len = 468/STRA = for | | | | | | | |
| 19 | 96829_at | Cluster Incl AW047083 UI-M-BH1-alr-f-05-0-UI s1 Mus musculus cDNA, 3 end/clone = UI-M-BH1-alr-f-05-0-UI/ | P | 817 | P | 573.7 | P | 721.8 | P |
| | | clone_end = 3/gb = AW047083/gi = 5907612/ug = Mm 29398/len = 311/STRA = rev | | | | | | | |
| 19 | 97424_at | Cluster Incl AW049647 UI-M-BH1-and-g-07-0-UI s1 Mus musculus cDNA, 3 end/clone = UI-M-BH1-and-g-07-0-UI/ | P | 452.3 | P | 499.4 | P | 871 | P |
| | | clone_end = 3/gb = AW049647/gi = 5910176/ug = Mm 30009/len = 487/STRA = for | | | | | | | |
| 19 | 98031_at | Cluster Incl AF027707 Mus musculus apoptosis activator Mtd (Mtd) mRNA, complete cds/cds = (188,829)/gb = AF027707/ | P | 619.4 | P | 698.3 | P | 683.2 | P |
| | | gi = 2689659/ug = Mm 3295/len = 1430/STRA = for | | | | | | | |
| 19 | 99027_at | Cluster Incl L35049 Bcl2-like/cds = (242,943)/gb = L35049/gi = 561740/ug = Mm 3882/len = 979/STRA = for | P | 403.6 | P | 482.1 | P | 688.5 | P |
| 19 | 99034_at | Cluster Incl Y15001 Mus musculus mRNA for Iroquois homeobox protein 3/cds = (0,1523)/gb = Y15001/gi = 2765437/ | P | 439.6 | P | 321.6 | P | 472.5 | P |
| | | ug = Mm 39039/len = 1937/STRA = for | | | | | | | |
| 19 | 100492_at | Cluster Incl AW122807: UI-M-BH2.1-aoy-h-10-0-UI s1 Mus musculus cDNA, 3 end/clone = UI-M-BH2 1-aoy-h-10-0-UI/ | P | 402.8 | P | 209.3 | P | 704.3 | P |
| | | clone_end = 3/gb = AW122807/gi = 6098337/ug = Mm 5063/len = 142/STRA = for | | | | | | | |
| 19 | 100515_at | Cluster Incl X54056 Proprotein convertase subtillsin/kexin type 3/cds = (94,2475)/gb = X54056/gi = 50996/ug = Mm 5241/ | P | 1102.5 | P | 1362.1 | P | 1450.3 | P |
| | | len/3978/STRA = rev | | | | | | | |
| 19 | 100986_at | Cluster Incl AF055889: Mus musculus LIM protein FHL2 (Fhl2) mRNA, complete cds/cds = (11,850)/gb = AF055889/ | P | 589.6 | P | 561.3 | P | 783.6 | P |
| | | gi = 3025852/ug = = Mm.6799/len = 1300/STRA = for | | | | | | | |
| 19 | 101457_at | Cluster Incl L16956: Janus Kinase 2/cds = (93,3482)/gb = L16956/gi = 309463/ug = Mm 809/len = 3629/STRA = for | P | 166.4 | P | 73.5 | P | 135.7 | P |
| 19 | 101836_at | Cluster Incl D45859 Protein phosphatase 1B, magnesium dependent, beta isoform/cds = (0,461)/gb = D45B59/gi = 961469/ | P | 71.2 | P | 94.3 | P | 103.8 | P |
| | | ug = Mm 849/len = 546/STRA = for | | | | | | | |
| 19 | 101876_s_at | Cluster Incl M35247 Histocompatibility 2, T region locus 17/cds = (34,168)/gb = M35247/gi = 199685/ug = Mm 88092/ | P | 376.5 | P | 187.2 | P | 247.1 | P |
| | | len = 1497/STRA = for | | | | | | | |
| 19 | 101881_g_at | Cluster Incl L22545 Procollagen, type XVIII, alpha 1/cds = (0,3866)/gb = L22545/gi = 348968/ug = Mm 88304/len = 4031/ | P | 470 | P | 415.9 | P | 318.4 | P |
| | | STRA = rev | | | | | | | |
| 19 | 102201_s_at | Cluster Incl L42177 Presenilin 1/cds = (187,1590)/gb = L42177/gi = 904129/ug = Mm 998/len = 1964/STRA = for | P | 830.2 | P | 947.9 | P | 839.9 | P |
| 19 | 102204_at | Cluster Incl L36435 Kreisler (maf-related) leucine zipper protein/cds = (169,1140)/gb = L35435/gi = 625043/ug = Mm 100216/ | P | 532.1 | P | 697.5 | P | 591 | P |
| | | len = 1458/STRA = for | | | | | | | |
| 19 | 103527_at | Cluster Incl AW124360 UI-M-BH2 1-apq-h-07-0-UI.s1 Mus musculus cDNA, 3 end/clone = UI-M-BH2.1-apq-h-07-0-UI/ | P | 74.6 | P | 138.9 | P | 205.2 | P |
| | | clone_end = 3/gb = AW124360/gi = 6099890/ug = Mm 23011/len = 232/STRA = rev | | | | | | | |
| 19 | 103542_at | Cluster Incl AW215059 up06a04 y1 Mus musculus cDNA, 5 end/clone = IMAGE-2651214/clone_end = 5/gb = AW215059/ | P | 746.6 | P | 902.5 | P | 702.3 | P |
| | | gi = 6525808/ug = Mm 23806/len = 245/STRA = rev | | | | | | | |
| 19 | 104217_at | Cluster Incl AW045753 UI-M-BH1-akt-a-10-0-UI s1 Mus musculus cDNA, 3 end/clone = UI-M-BH1-akt-a-10-0-UI/ | P | 294.1 | P | 258.4 | P | 382.8 | P |
| | | clone_end = 3/gb = AW045753/gi = 5906282/ug = Mm.27893/len = 407/STRA = rev | | | | | | | |
| 19 | 104221_at | Cluster Incl AB017189 Mus musculus mRNA for 4F2/CD98 light chain, complete cds/cds = (26,1564)/gb = AB017189/ | P | 772.4 | P | 920.8 | P | 669 | P |
| | | gi = 4519255/ug = Mm 27943/len = 3441/STRA = for | | | | | | | |
| 19 | 104241_at | Cluster Incl AI854379: UI-M-BG1-aic-f-11-0-UI s1 Mus musculus cDNA, 3 end/clone = UI-M-BG1-aic-f-11-0-UI/ | P | 1017.4 | P | 792.5 | P | 1500.8 | P |
| | | clone_end = 3/gb = AI854379/gi = 5498285/ug = Mm.28184/len = 374/STRA = rev | | | | | | | |
| 19 | 104514_at | Cluster Incl AF057285: Mus musculus intersectin-EH binding protein Ibp1 mRNA, partial cds/cds = (0,639)/gb = AF057285/ | P | 1735 | P | 2270.5 | P | 2035.5 | P |
| | | gi = 3063646/ug = Mm 3091/len = 1048/STRA = for | | | | | | | |
| 19 | 92245_at | Cluster Incl AI642262 vn75a09 y1 Mus musculus cDNA, 5 end/clone = IMAGE-1037752/clone_end = 5/gb = AI642262/ | P | 180.3 | P | 326.2 | P | 355.4 | P |
| | | gi = 4720737/ug = Mm 35019/len = 255/STRA = rev | | | | | | | |
| 19 | 93195_at | Cluster Incl AI957146 ul76b03 x1 Mus musculus cDNA, 3 end/clone = IMAGE-2136461/clone_end = 3/gb = AI957146/ | P | 296.3 | P | 335 | P | 327 | P |
| | | gi = 5749855/ug = Mm 5601/len = 465/STRA = for | | | | | | | |
| 19 | 93624_at | Cluster Incl AI225296 ue86b01 y1 Mus musculus cDNA, 5 end/clone = IMAGE-1497961/clone_end = 5/gb = AI225296/ | P | 187.9 | P | 144.3 | P | 290.4 | P |
| | | gi = 3808357/ug = Mm 7446/len = 418/STRA = for | | | | | | | |
| 19 | 93673_at | Cluster Incl U78109 Neurturin/cds = (348,935)/gb = U78109/gi = 1731678/ug = Mm 8074/len = 1023/STRA = for | P | 597.2 | P | 797.2 | P | 946.2 | P |
| 19 | 93712_at | Cluster Incl AF095640 Mus musculus cyclin T1 mRNA, complete cds/cds = (0,2174)/gb = AF095640/gi = 4106516/ | P | 285.2 | P | 414.8 | P | 458.6 | P |
| | | ug = Mm 86538/len = 2175/STRA = for | | | | | | | |
| 19 | 94192_at | Cluster Incl Y17860 Mus musculus mRNA for ganglioside-Induced differentiation associated protein 10/cds = (2531,2917)/ | P | 216 | P | 282.3 | P | 282.1 | P |
| | | gb = Y17860/gi = 3378471/ug = Mm 12950/len = 5011/STRA = for | | | | | | | |
| 19 | 94794_at | Cluster Incl X52561: Mouse gene for ferritin H subunit/cds = (166,714)/gb = X52561/gi = 50953/ug = Mm 1776/len = 860/ | P | 3757.3 | P | 3879.1 | P | 3786.1 | P |
| | | STRA = for | | | | | | | |
| 19 | 96596_at | Cluster Incl U52073 Mus musculus TDD5 mRNA, complete cds/cds = (135,1280)/gb = U52073/gi = 1903378/ug = Mm 30837/ | P | 547 | P | 490.9 | P | 581.1 | P |
| | | len = 2916/STRA = for | | | | | | | |
| 19 | 97786_at | Cluster Incl AJ011118 Mus musculus mRNA for a skeletal muscle and cardiac protein/cds = (10,996)/gb = AJ011118/ | P | 315.2 | P | 241.7 | P | 311.4 | P |
| | | gi = 5420271/ug = Mm 41992/len = 1100/STRA = for | | | | | | | |
| 19 | 98868_at | Cluster Incl 31532 B-cell leukemia/lymphoma 2/cds = (1197,1796)/gb = L31532/gi = 468337/ug = Mm 5155/len = 1897/ | P | 89.4 | P | 113.7 | P | 169.3 | P |
| | | STRA = for | | | | | | | |
| 19 | 99849_at | Cluster Incl C85523 C85523 Mus musculus cDNA, 3 end/clone = J0209F01/clone_end = 3/gb = C85523/gi = 2917480/ | P | 2870.2 | P | 1278.3 | P | 1890.1 | P |
| | | ug = Mm 63525/len = 566/STRA = rev | | | | | | | |
| 19 | 100332_s_at | Cluster Incl AF093853 Mus musculus 1-Cys peroxiredoxin protein 2 gene, complete cds/cds = (0,674)/gb = AF093853/ | P | 402.5 | P | 276.4 | P | 497.7 | P |
| | | gi = 3769943/ug = Mm 87841/len = 675/STRA = for | | | | | | | |
| 19 | 95796_g_at | Cluster Incl U96810 Mus musculus Sup14h2 gene, complete cds/cds = (0,353)/gb = U96810/gi = 3779195/ug = Mm 87825/ | P | 587.8 | P | 731.6 | P | 773.8 | P |
| | | len = 354/STRA = for | | | | | | | |
| 19 | 96391_at | Cluster Incl C80836 C80836 Mus musculus cDNA, 3 end/clone = J0089A10/clone_end = 3/gb = C80836/gi = 2521166/ | P | 241.1 | P | 206.6 | P | 350.8 | P |
| | | ug = Mm.25089/len = 401/STRA = rev | | | | | | | |
| 19 | 97601_f_at | Cluster Incl AW259538: up30a03 x1 Mus musculus cDNA, 3 end/clone = IMAGE-2655820/clone_end = 3/gb = AW259538/ | P | 2327.7 | P | 2289.1 | P | 2591.5 | P |
| | | gi = 6632519/ug = Mm 89688/len = 374/STRA = rev | | | | | | | |
| 19 | 93492_at | Cluster Incl AB013469 Mus musculus CLM2 gene for cytohesin 2, complete and partial cds, alternative splicing/ | P | 762.4 | P | 889.7 | P | 911.3 | P |
| | | cds = (0,1154)/gb = AB013469/gi = 3660542/ug = Mm 19097/len = 1155/STRA = for | | | | | | | |
| 19 | 94299_at | Cluster Incl AI839731: UI-M-AP0-abe-a-03-0-UI s2 Mus musculus cDNA, 3 end/clone = UI-M-AP0-abe-a-03-0-UI/ | P | 245.1 | P | 213.9 | P | 265.6 | P |
| | | clone_end = 3/gb = AI839731/gi = 5473939/ug = Mm 22597/len = 359/STRA = for | | | | | | | |
| 19 | 97915_at | Cluster Incl AW124144: UI-M-BH2 1-apr-e-12-0-UI s1 Mus musculus cDNA, 3 end/clone = UI-M-BH2 1-apr-e-12-0-UI/ | P | 406.1 | P | 399.9 | A | 578.5 | P |
| | | clone_end = 3/gb = AW124144/gi = 6099674/ug = Mm 28491/len = 442/STRA = for | | | | | | | |
| 19 | 99677_at | Cluster Incl AI849559: UI-M-AH1-agr-b-09-0-UI s1 Mus musculus cDNA, 3 end/clone = UI-M-AH1-agr-b-09-0-UI/ | P | 293.6 | P | 321.4 | P | 427.7 | P |
| | | clone_end = 3/gb = AI849559/gi = 5493465/ug = Mm 44218/len = 371/STRA = for | | | | | | | |
| 19 | 104562_at | Cluster lncl AW049632: UI-M-BH1-and-f-02-0-UI.s1 Mus musculus cDNA, 3 end/clone = UI-M-BH1-and-f-02-0-UI/ | P | 226.6 | P | 236.8 | P | 398.9 | A |
| | | clone_end = 3/gb = AW049632/gi = 5910181/ug = Mm 23838/len = 385/STRA = for | | | | | | | |
| 19 | 96205_at | Cluster Incl AW048272: UI-M-BH1-aly-e-02-0-UI s1 Mus musculus cDNA, 3 end/clone = UI-M-BH1-aly-e-02-0-UI/ | P | 127.7 | P | 346 | P | 275.9 | P |
| | | clone_end = 3/gb = AW048272/gi = 5908801/ug = Mm.28638/len = 465/STRA = for | | | | | | | |
| 19 | 97319_at | Cluster Incl AF084466 Mus muaculus Ras-like GTP-binding protein Red mRNA, complete cds/cds = (8,932)/gb = AF084466/ | P | 289.3 | P | 138 | A | 363.8 | P |
| | | gi = 3462897/ug = Mm 29467/len = 934/STRA = for | | | | | | | |
| 19 | 97363_at | Cluster Incl M95408 Focal adhesion kinase/cds = (109,3267)/gb = M95408/gi = 193223/ug = Mm 29674/len = 4211/STRA = for | P | 54.3 | P | 90.9 | P | 33.7 | P |
| 19 | 97398_at | Cluster Incl AI849831: UI-M-AL1-aho-h-05-0-UI s1 Mus musculus cDNA, 3 end/clone = UI-M-AL1-aho-h-05-0-UI/ | P | 130.3 | P | 131.8 | A | 176.8 | P |
| | | clone_end = 3/gb = AI849831/gi = 5493777/ug = Mm 29856/len = 362/STRA = for | | | | | | | |
| 19 | 97413_at | Cluster Incl AI121305: uc30b06.r1 Mus musculus cDNA, 5 end/clone = IMAGE-1399475/clone_end = 5/gb = AI121305/ | P | 259.3 | P | 262.4 | P | 206.2 | P |
| | | gi = 3521629/ug = Mm.29959/len = 358/STRA = for | | | | | | | |
| 19 | 98472_at | Cluster Incl Y00629: Histocompatibility 2, T region locus 23/cds = (32,1105)/gb = Y00629/gi = 51013/ug = Mm.35016/ | P | 481.3 | P | 150 | A | 443.3 | P |
| | | len = 1282/STRA = for | | | | | | | |
| 19 | 102627_at | Cluster Incl AF061569 Mus musculus coding region determinant binding protein mRNA, complete cds/cds = (130,1863)/ | P | 133.4 | P | 181.9 | P | 195.1 | P |
| | | gb = AF061569/gi = 3273748/ug = Mm 12919/len = 2223/STRA = for | | | | | | | |
| 19 | 104201_at | Cluster Incl AA718649 vt86g08 r1 Mus musculus cDNA, 5 end/clone = IMAGE-1178078/clone_end = 5/gb = AA718649/ | P | 657.9 | P | 288.3 | P | 687.2 | P |
| | | gi = 2730923/ug = Mm 27525/len = 479/STRA = for | | | | | | | |
| 19 | 92189_at | Cluster Incl AI846017: UI-M-AP1-agl-f-08-0-UI s1 Mus musculus cDNA, 3 end/clone = UI-M-AP1-agl-f-08-0-UI/clone_end = 3/ | P | 148.7 | P | 83.6 | P | 203.3 | P |
| 19 | 99397_at | Cluster Incl U97572 Excision repair 2/cds = (0,2282)/gb = U97572/gi = 2114483/ug = Mm 56990/len = 2283/STRA = for | P | 250.9 | P | 352.6 | M | 426.9 | P |
| 19 | 99915_at | Cluster Incl L41352 Amphiregulin/cds = (0,746)/gb = L41352/gi = 845598/ug = Mm.8039/len = 747/STRA = for | P | 74.7 | P | 85.8 | P | 79.5 | A |
| 19 | 101676_at | Cluster Incl U13705 Glutathione peroxidase 3/cds = (16,696)/gb = U13705/gi = 536927/ug = Mm 7156/len = 1415/STRA = for | P | 544 | P | 319.4 | P | 791.1 | P |
| 19 | 95095_at | Cluster Incl U90435 Mus musculus flotillin mRNA, complete cds/cds = (159,1445)/gb = U90435/gi = 2149603/ug = Mm.2931/ | P | 131.2 | P | 110.2 | A | 283 | A |
| | | len = 1702/STRA = for | | | | | | | |
| 19 | 101014_at | Cluster Incl Y09864 M musculus mRNA for type I interferon receptor, IFNaR2b/cds = (220,963)/gb = Y09864/gi = 2467245/ | P | 118 | P | 133.5 | M | 123 | P |
| | | ug = Mm 6834/len = 2586/STRA = for | | | | | | | |
| 19 | 103053_at | Cluster Incl X15784 Myogenin/cds = (48,788)/gb = X15784/gi = 53303/ug = Mm 16528/len = 1474/STRA = for | P | 217.6 | P | 91.3 | A | 247.9 | P |
| 19 | 103335_at | Cluster Incl U55060 Mus musculus beta-galactoside binding lectin mRNA, complete cds/cds = (34,1002)/gb = U55060/ | P | 796 | P | 216.9 | A | 683.5 | M |
| | | gi = 1916601/ug = Mm 18087/len = 1418/STRA = for | | | | | | | |
| 19 | 103557_at | Cluster Incl AI850046 UI-M-BG0-aia-b-11-0-U1 s1 Mus musculus cDNA, 3 end/clone = UI-M-BG0-aia-b-11-0-UI/ | P | 367 | P | 418.9 | P | 427.4 | A |
| | | clone_end = 3/gb = AI850046/gi = 5493952/ug = Mm 19091/len = 484/STRA = for | | | | | | | |
| 19 | 104152_at | Cluster Incl AA960466 ub58f11 s1 Mus musculus cDNA, 3 end/clone = IMAGE-1381965/clone_end = 3/gb = AA960466/ | P | 772.4 | P | 570.8 | M | 877.3 | M |
| | | gi = 3126366/ug = Mm 22193/len = 202/STRA = for | | | | | | | |
| 19 | 103737_at | Cluster Incl AB016780 Mus musculus mRNA for Glutamine-fructose-6-phosphate amidotransferase 2, complete cds/ | P | 60.1 | P | 240.6 | M | 328.6 | P |
| | | cds = (48,2096)/gb = AB016780/gi = 4239878/ug = Mm 24402/len = 2958/STRA = for | | | | | | | |
| 19 | 104645_at | Cluster Incl AI853712.UI-M-BH0-ajq-d-05-0-UI s1 Mus musculus cDNA, 3 end/clone = UI-M-BH0-ajq-d-05-0-UI/ | P | 233.3 | P | 384.2 | P | 307.3 | P |
| | | clone_end = 3/gb = AI853712/gi = 5497583/ug = Mm 31321/len = 496/STRA = rev | | | | | | | |
| 19 | 93865_s_at | Cluster Incl M35244: Histocompatibility 2, T region locus 10/cds = UNKNOWN/gb = M35244/gi = 199063/ug = Mm.87776/ | P | 406.4 | P | 313 | P | 400 | P |
| | | len = 1714/STRA = for | | | | | | | |
| 19 | 95910_f_at | Cluster Incl AI844780 UI-M-AL1-ahq-g-08-0-UI s1 Mus musculus cDNA, 3 end/clone = UI-M-AL1-ahq-g-08-0-UI/ | P | 218.7 | P | 245.9 | A | 291.1 | A |
| | | clone_end = 3/gb = AI844780/gi = 5488686/ug = Mm 24440/len = 200/STRA = for | | | | | | | |
| 19 | 93283_at | Cluster Incl AA869401 vq07f04 r1 Mus musculus cDNA, 5 end/clone = IMAGE-1093567/clone_end = 5/gb = AA869401/ | A | 340.7 | P | 242.7 | A | 593.2 | P |
| | | gi = 2964846/ug = Mm 1786/len = 793/STRA = for | | | | | | | |
| 19 | 96646_at | Cluster Incl AI846253 UI-M-AK1-aez-b-10-0-UI s1 Mus musculus cDNA, 3 end/clone = UI-M-AK1-aez-b-10-0-UI/ | A | 154.3 | A | 158.3 | P | 214.4 | M |
| | | clone_end = 3/gb = AI846253/gi = 5490159/ug = Mm 27292/len = 311/STRA = for | | | | | | | |
| 19 | 99100_at | Cluster Incl AI837104 UI-M-AK0-adc-d-07-0-UI s1 Mus musculus cDNA, 3 end/clone = UI-M-AK0-adc-d-07-0-UI/ | P | 448.5 | P | 361 | A | 418.4 | M |
| | | clone_end = 3/gb = AI837104/gi = 5471317/ug = Mm 3948/len = 413/STRA = for | | | | | | | |
| 19 | 101956_at | Cluster Incl AI834849 UI-M-AN0-acm-d-08-0-UI s2 Mus musculus cDNA, 3 end/clone = UI-M-AN0-acm-d-08-0-UI/ | P | 627.4 | P | 781 | A | 829.3 | A |
| | | clone_end= 3/gb = AI834849/gi = 5469062/ug = Mm 9210/len = 176/STRA = for | | | | | | | |
| 19 | 102849_at | Cluster Incl D88159 Potassium inwardly rectifying channel, subfamily J, member 8/cds = (203,1477)/gb = D88159/ | P | 134 | P | 132.7 | P | 186.4 | P |
| | | gi = 1881405/ug = Mm 1482/len = 1712/STRA = for | | | | | | | |
| 19 | 101934_at | Cluster Incl AI851119 UI-M-BH0-ajx-a-08-0-UI s1 Mus musculus cDNA, 3 end/clone = UI-M-BH0-ajx-a-08-0-UI/ | P | 68.5 | A | 55.3 | P | 105.6 | P |
| | | clone_end = 3/gb = AI851119/gi = 5495065/ug = Mm.946/len = 354/STRA = for | | | | | | | |
| 19 | 94147_at | Cluster Incl M33960: Mouse plasminogen activator inhibitor (PAI-1) mRNA, complete cds/cds = (131,1339)/gb = M33960/ | P | 132.5 | P | 79 | A | 224.9 | P |
| | | gi = 200219/ug = Mm.1263/len = 2999/STRA = for | | | | | | | |
| 19 | 96418_r_at | Cluster Incl M18399 Mouse homeo box (Hox2.4) region/cds = (0,389)/gb = M18399/gi = 193940/ug = Mm 88400/len = 390/ | P | 396 | P | 659.2 | P | 426.5 | A |
| | | STRA = for | | | | | | | |
| 19 | 93352_at | Cluster Incl M55154.Mouse transglutaminase (TGase) mRNA, complete cds/cds = (29,2086)/gb = M55154/gi = 201940/ | P | 88.1 | P | 110.7 | A | 154.4 | P |
| | | ug = Mm 18843/len = 3483/STRA = for | | | | | | | |
| 19 | 100552_at | Cluster Incl M28233: Mouse interferon-gamma receptor mRNA, complete cds/cds = (101,1534)/gb = M28233/gi = 194131/ | A | 217.4 | P | 136.6 | A | 226.7 | P |
| | | ug = Mm 549/len = 2093/STRA = for | | | | | | | |
| 19 | 102845_at | Cluster Incl A1836034 UI-M-AQ0-aad-f-06-0-UI s2 Mus musculus cDNA, 3 end/clone = Ul-M-AQ0-aad-f-06-0-UI/ | P | 285.3 | P | 345 | P | 328.1 | M |
| | | clone_end = 3/gb = AI838034/gi = 5470212/ug = Mm 14741/len = 389/STRA = rev | | | | | | | |
| 19 | 102988_at | Cluster Incl U92477: Mus musculus AblSH3 binding protein mRNA, partial cds/cds = (0,1440)/gb = U92477/gi = 1928963/ | A | 790.1 | P | 842.3 | P | 881.9 | P |
| | | ug = Mm 1531/len = 2188/STRA = for | | | | | | | |
| 19 | 104548_at | Cluster Incl Y15443 Mus musculus mRNA for 50C15 protein/cds = (20,454)/gb = Y15443/gi = 2624230/ug = Mm 23697/ | P | 166 | P | 187.5 | A | 176.3 | A |
| | | len = 721/STRA = for | | | | | | | |
| 19 | 94423_at | Cluster Incl AW049551 UI-M-BH1-anf-f-04-0-UI s1 Mus musculus cDNA, 3 end/clone UI-M-BH1-anf-f-04-0-UI/ | P | 90.6 | IP | 65.1 | M | 82.1 | A |
| | | clone_end = 3/gb = AW049551/gi = Mm 26282/len = 447/STRA = for | | | | | | | |
| 19 | 94997_at | Cluster Incl AF060883 Mus musculus endomucin mRNA, complete cds/cds = (97,843)/gb = AF060883/gi = 4159992/ | P | 241.9 | P | 165.2 | M | 200.3 | A |
| | | ug = Mm.27343/len = 1374/STRA = for | | | | | | | |
| 19 | 96167_at | Cluster Incl AI643420: vv24h01.x1 Mus musculus cDNA, 3 end/clone = IMAGE-1223377/clone_end = 3/gb = AI643420/ | P | 437 | P | 536 | P | 350.8 | A |
| | | gi = 4721895/ug = Mm 28373/len = 195/STRA = for | | | | | | | |
| 19 | 104268_at | Cluster Incl X51975: Interleukin 6 receptor, alpha/cds = (0,1382)/gb = X51975/gi = 49725/ug = Mm 2856/len = 2499/STRA = for | P | 67.9 | A | 21.5 | A | 306.9 | P |
| 19 | 99916_at | Cluster Incl D90242: Protein kinase C, eta/cds = (59,2110)/gb = D90242/gi = 220526/ug = Mm 8040/len = 2176/STRA = for | P | 49.1 | A | 103 | A | 126.1 | P |
| 19 | 94288_at | Cluster Incl J03482 Mouse histone H1 gene, complete cds/cds = (42,680)/gb = J03482/gi = 193847/ug = Mm 22570/len = 725/ | P | 588.3 | A | 353.1 | P | 1039.7 | P |
| | | STRA = for | | | | | | | |
| 19 | 102430_at | Cluster Incl X51397: Myeloid differentiation primary response gene 88/cds = (109,840)/gb = X51397/gi = 53293/ | P | 182.5 | P | 104.1 | A | 199.7 | A |
| | | ug = Mm.12847/len = 1805/STRA = for | | | | | | | |
| 19 | 103367_at | Cluster Incl U18975 Ganglioside expression 2/cds = (3,1604)/gb = U18975/gi = 945034/ug = Mm 1853/len = 2109/STRA = for | P | 61.3 | P | −15.4 | A | 86 | A |
| 19 | 93108_at | Cluster Incl J05185 Prolyl 4-hydroxylase, beta polypeptide/cds = (129,1658)/gb = J05185/gi = 200280/ug = Mm.16660/ | P | 383.7 | A | 355.8 | P | 354.9 | A |
| | | len = 2537/STRA = for | | | | | | | |
| 19 | 94902_at | Cluster Incl U38261 Mus musculus extracellular superoxide dismutase (SOD3) mRNA, complete cds/cds = (269,1024)/ | A | 265.3 | P | 411.5 | A | 468.4 | A |
| | | gb = U38261/gi = 1915962/ug = Mm 2407/len = 1834/STRA = for | | | | | | | |
| 19 | 97830_at | Cluster Incl L06039 Platelet/endothelial cell adhesion molecule/cds = (98,2281)/gb = L06039/gi = 453288/ug = Mm.2822/ | P | 159 | A | 211.9 | A | 254.9 | A |
| | | len = 2534/STRA = for | | | | | | | |
| 19 | 102046_at | Cluster Incl AF041847 Mus musculus cardiac ankynn repeal protein MCARP mRNA, complete cds/cds = (14,973)/ | P | 25.9 | A | 17.7 | A | 23.1 | A |
| | | gb = AF041847/gi = 2905615/ug = Mm 10279/len = 1026/STRA = for | | | | | | | |
| 19 | 103068_at | Cluster Incl U68535: Mus musculus aldo-keto reductase mRNA, complete cds/cds = (27,932)/gb = U68535/gi = 1698717/ | A | −22.9 | P | 52.4 | P | 138.5 | A |
| | | ug = Mm 16790/len = 1675/STRA = for | | | | | | | |
| 19 | 102195_at | Cluster Incl U88984: Mus musculus NIK mRNA, complete cds/cds = (0,3701)/gb = U88984/gi = 1872545/ug = Mm 987/ | P | 145.2 | P | 169.8 | A | 139.4 | A |
| | | len = 3915/STRA = rev | | | | | | | |
| 19 | 103213_at | Cluster Incl AJ245402: Mus musculus partial cdh15 gene for cadherin/cds = (0,239)/gb = AJ245402/gi = 6010257/ | P | 42.6 | A | 154.8 | A | 170.6 | P |
| | | ug = Mm 1976/len = 240/STRA = for | | | | | | | |
| 19 | 92738_at | Cluster Incl D49921: Mouse mRNA for glial cell line-derived neurotrophic factor(GDNF), complete cds/cds = (0,635)/ | P | 201.7 | P | 217.1 | P | 210.3 | P |
| | | gb = D49921/gi = 758584/ug = Mm 4679/len = 641/STRA = for | | | | | | | |
| 19 | 97166_at | Cluster Incl W49316: mc89e12 r1 Mus musculus cDNA, 5 end/clone = IMAGE-355726/clone_end = 5/gb = W49316/ | P | 37.6 | A | 44.2 | A | 47.7 | A |
| | | gi = 1337600/ug = Mm.35552/len = 475/STRA = rev | | | | | | | |
| 19 | 94643_at | Cluster Incl U35836: Tumor-associated antigen 1/cds = (10,1257)/gb = U35836/gi = 1017751/ug = Mm 8071/len = 1338/ | A | 54.9 | P | 89 | M | 87.5 | A |
| | | STRA = for | | | | | | | |
| 20 | 92540_f_at | Cluster Incl Z67748 M musculus spermidine synthase gene/cds = (75,983)/gb = Z67748/gi = 1061191/ug = Mm.10/len = 1261/ | P | 315.7 | P | 221.5 | P | 283.3 | P |
| | | STRA = for | | | | | | | |
| 20 | 92770_at | Cluster Incl X66449 Calcyclin/cds = (159,428)/gb = X66449/gi = 50271/ug = Mm 1221/len = 551/STRA = for | P | 4480.8 | P | 5132.7 | P | 4814.9 | P |
| 20 | 92817_at | Cluster Incl AW120646 UI-M-BH2 3-anz-f-08-0-UI s1 Mus musculus cDNA, 3 end/clone = UI-M-BH2.3-anz-f-08-0-UI/ | P | 837.2 | P | 852.8 | P | 842.4 | P |
| | | clone_end = 3/gb = AW120646/gi = 6096019/ug = Mm 12864/len = 434/STRA = for | | | | | | | |
| 20 | 92861_i_at | Cluster Incl M13805 Mouse type I epidermal keratin mRNA, clone pkSCC-50, 3 end/cds = (0,617)/gb = M13805/gi = 198632/ | P | 2980.7 | P | 3940.5 | P | 3339 | P |
| | | ug = Mm 14046/len = 801/STRA = for | | | | | | | |
| 20 | 92862_f_at | Cluster Incl M13805 Mouse type I epidermal keratin mRNA, clone pkSCC-50, 3 end/cds-(0,617)/gb = M13805/gi = 198632/ | P | 3772.1 | P | 4355.1 | P | 3449 | P |
| | | ug = Mm.14046/len = 801/STRA = for | | | | | | | |
| 20 | 92877_at | Cluster Incl L19932 Transforming growth factor, beta induced, 68 kDa/cds = (45,2096)/gb = L19932/gi = 451131/ | P | 928.2 | P | 523 | P | 709.3 | P |
| | | ug = Mm 14455/len = 2674/STRA = for | | | | | | | |
| 20 | 93018_at | Cluster Incl AI790103 ue64f04 r1 Mus musculus cDNA, 5 end/clone = IMAGE-1495903/clone_end = 5/gb = AI790103/ | P | 975.5 | P | 888.7 | P | 596 | P |
| | | gi = 5337819/ug = Mm 14747/len = 346/STRA = for | | | | | | | |
| 20 | 93042_at | Cluster Incl D21207 Benzodiazepine receptor, peripheral/cds = (65,574)/gb = D21207/gi = 484053/ug = Mm 1508/len = 856/ | P | 2304 | P | 1956.9 | P | 2048 | P |
| | | STRA = for | | | | | | | |
| 20 | 93078_at | Cluster Incl X04653 Mouse mRNA for Ly-6 alloantigen (Ly-6E 1)/cds = (88,492)/gb = X04653/gi = 52958/ug = Mm 15889/ | P | 1883.3 | P | 1787.5 | P | 1899.8 | P |
| | | len = 876/STRA = for | | | | | | | |
| 20 | 93264_at | Cluster Incl AI843895 UI-M-AK1-aeu-f-09-0-UI s1 Mus musculus cDNA, 3 end/clone = UI-M-AK1-aeu-f-09-0-UI/ | P | 816.8 | P | 669.2 | P | 631.7 | P |
| | | clone_end = 3/gb = AI843895/gi = 5478108/ug = Mm 17253/len = 420/STRA = for | | | | | | | |
| 20 | 93288_at | Cluster Incl AI835883 UI-M-AI0-aat-f-08-0-UI.s1 Mus musculus cDNA, 3 end/clone = UI-M-AI0-aat-f-08-0-UI/clone_end = 3/ | P | 2873.3 | P | 3601.7 | P | 2845.5 | P |
| | | gb = AI835883/gi = 5470096/ug = Mm 17912/len = 426/STRA = for | | | | | | | |
| 20 | 93362_at | Cluster Incl U27106 Adapter-related protein complex AP-2, mu1/cds = (141,1448)/gb = U27106/gi = 2065471/ug = Mm.18946/ | P | 1743.3 | P | 1950.8 | P | 1801.5 | P |
| | | len = 2066/STRA = rev | | | | | | | |
| 20 | 93573_at | Cluster Incl V00835 Metallothionein 1/cds = (0,185)/gb = V00835/gi = 53247/ug = Mm 2041/len = 186/STRA = for | P | 2074.9 | P | 2326.5 | P | 1420.3 | P |
| 20 | 93586_at | Cluster Incl AJ002307: Mus musculus mRNA for synaptogyrin 2/cds = (4,678)/gb = AJ002307/gi = 2959869/ug = Mm.2079/ | P | 296.5 | P | 202.8 | P | 104.6 | P |
| | | len = 1512/STRA = for | | | | | | | |
| 20 | 93605_r_at | Cluster Incl AF061260 Mus musculus immunosuperfamily protein B12 mRNA, complete cds/cds = (308,1195)/ | P | 188.1 | P | 239.8 | P | 215.1 | P |
| | | gb = AF081260/gi = 3779241/ug = Mm 20921/len = 1861/STRA = for | | | | | | | |
| 20 | 93797_g_at | Cluster Incl AW123952 UI-M-BH2 3-aqc-d-05-0-UI s1 Mus musculus cDNA, 3 end/clone = UI-M-BH2 3-aqc-d-05-0-UI/ | P | 336.6 | P | 372.1 | P | 409 | P |
| | | clone_end = 3/gb = AW123952/gi = 6099482/ug = Mm 2140/len = 370/STRA = for | | | | | | | |
| 20 | 94011_at | Cluster Incl AI845182 UI-M-BG0-aht-a-11-0-UI s1 Mus musculus cDNA, 3 end/clone = UI-M-BG0-aht-a-11-0-UI/ | P | 1429.3 | P | 909 | P | 865.1 | P |
| | | clone_end = 3/gb = AI845182/gi = 5489088/ug = Mm 21819/len = 325/STRA = for | | | | | | | |
| 20 | 94043_at | Cluster Incl AB031290 Mus musculus mRNA for C7-1 protein, complete cds/cds = (0,1391)/gb = AB031290/gi = 5771446/ | P | 604.2 | P | 586.9 | P | 540.9 | P |
| | | ug = Mm 21961/len = 1524/STRA = for | | | | | | | |
| 20 | 94054_at | Cluster Incl AI841808: UI-M-AN0-acl-h-05-0-UI s1 Mus musculus cDNA, 3 end/clone = UI-M-AN0-acl-h-05-0-UI/ | P | 888.2 | P | 638.9 | P | 532.4 | P |
| | | clone_end = 3/gb = AI841808/gi = 5476021/ug = Mm 22024/len = 173/STRA = for | | | | | | | |
| 20 | 94302_at | Cluster Incl AF013099 Mus musculus multiubiquitin-chain-binding protein (Mcb1) mRNA, complete cds/cds = (12,1142)/ | P | 983.1 | P | 1019.7 | P | 1036.9 | P |
| | | gb = AF013099/gi = 2293572/ug = Mm 2261/len = 1252/STRA = for | | | | | | | |
| 20 | 94826_at | Cluster Incl Y11460 Mus musculus mRNA for b4 Integnn interactor, partial/cds = (0,254)/gb = Y11460/gi = 2910994/ | P | 827.6 | P | 1046.2 | P | 1013.7 | P |
| | | ug = Mm 22731/len = 438/STRA = for | | | | | | | |
| 20 | 94831_at | Cluster Incl M65270 Mouse cathepsin B gene/cds = (25,1044)/gb = M65270/gi = 309151/ug = Mm.22753/len = 1848/ | P | 2442.8 | P | 2686.7 | P | 1956.1 | P |
| | | STRA = for | | | | | | | |
| 20 | 94835_f_at | Cluster Incl M28739 Tubulin, beta 2/cds = (0,302)/gb = M28739/gi = 202226/ug = Mm 22774/len = 488/STRA = for | P | 1348.5 | P | 1258.8 | P | 1391.7 | P |
| 20 | 94837_at | Cluster Incl U67328 Mus musculus NIPI-like protein (NIPIL(A3)) mRNA, complete cds/cds = (466,1437)/gb = U67328/ | P | 2537.1 | P | 2720.2 | P | 2922.3 | P |
| | | gi = 1762985/ug = Mm 22776/len = 1720/STRA = for | | | | | | | |
| 20 | 95406_at | Cluster Incl AW125347 UI-M-BH2 1-apy-h-03-0-UI s1 Mus musculus cDNA, 3 end/clone = UI-M-BH2 1-apy-h-03-0-UI/ | P | 1700.9 | P | 696.1 | P | 738.8 | P |
| | | clone_end = 3/gb = AW125347/gi = 6100877/ug = Mm 24219/len = 331/STRA = for | | | | | | | |
| 20 | 95417_at | Cluster Incl AI117848: uc41c02.r1 Mus musculus cDNA, 5 end/clone = IMAGE-1400546/clone_end = 5/gb = AI117848/ | P | 281.4 | P | 322.7 | P | 231.5 | P |
| | | gi = 3518172/ug = Mm 24293/len = 431/STRA = for | | | | | | | |
| 20 | 95433_at | Cluster Incl AW049776 UI-M-BH1-anm-c-12-0-UI s1 Mus musculus cDNA, 3 end/clone = UI-M-BH1-anm-c-12-0-UI/ | P | 337.3 | P | 344.8 | P | 294.9 | P |
| | | clone_end = 3/gb = AW049776/gi = 5910305/ug = Mm.24495/len = 403/STRA = for | | | | | | | |
| 20 | 95511_at | Cluster Incl X69902 Integrin alpha 6/cds = (181,3402)/gb = X69902/gi = 408127/ug = Mm 25232/len = 3756/STRA = for | P | 332.2 | P | 225.3 | P | 177.9 | P |
| 20 | 96033_at | Cluster Incl Z22532 Syndecan 1/cds = (240,1175)/gb = Z22532/gi = 1946227/ug = Mm 2580/len = 3054/STRA = for | P | 297 | P | 363 | P | 269.2 | P |
| 20 | 96042_at | Cluster Incl L35528: Mus musculus manganese superoxide dismutase (MnSOD) gene/cds = (70,738)/gb = L35528/ | P | 347.7 | P | 243.3 | P | 283 | P |
| | | gi = 2182263/ug = Mm 2597/len = 906/STRA = for | | | | | | | |
| 20 | 96075_at | Cluster Incl AW060876 UI-M-BH1-amx-g-07-0-UI s1 Mus musculus cDNA, 3 end/clone = UI-M-BH1-amx-g-07-0-UI/ | P | 1154.2 | P | 1209.6 | P | 1027.8 | P |
| | | clone_end = 3/gb = AW060876/gi = 6008627/ug = Mm 2654/len = 472/STRA = for | | | | | | | |
| 20 | 96092_at | Cluster Incl M96827 C57BL/6J ob/ob haptoglobin mRNA, complete cds/cds = (48,1091)/gb = M96827/gi = 193753/ | P | 2789.1 | P | 1247.2 | P | 921.4 | P |
| | | ug = Mm 26730/len = 1267/STRA = for | | | | | | | |
| 20 | 96603_at | Cluster Incl AW123556 UI-M-BH2 1-aqb-g-04-0-UI s1 Mus musculus cDNA, 3 end/clone = UI-M-BH2 1-aqb-g-04-0-UI/ | P | 351.3 | P | 247.7 | P | 363.6 | P |
| | | clone_end = 3/gb = AW123556/gi = 6099086/ug = Mm 27035/len = 370/STRA = for | | | | | | | |
| 20 | 96623_at | Cluster Incl AI853172 UI-M-BH0-ajh-b-06-0-UI s1 Mus musculus cDNA, 3 end/clone = UI-M-BH0-ajh-b-06-0-UI/ | P | 613 | P | 173.3 | P | 255.2 | P |
| | | clone_end = 3/gb = AI853172/gi = 5497078/ug = Mm 27173/len = 435/STRA = for | | | | | | | |
| 20 | 96650_at | Cluster Incl AI837724: UI-M-AK0-adj-f-11-0-UI s1 Mus musculus cDNA, 3 end/clone = UI-M-AK0-adj-f-11-0-UI/clone_end = 3/ | P | 220.5 | P | 213 | P | 262 | P |
| | | gb = AI837724/gi = 5471937/ug = Mm 2733/len = 370/STRA = for | | | | | | | |
| 20 | 96657_at | Cluster Incl L10244 Spermidine/spermine N1-acetyl transferase/cds = (89,604)/gb = L10244/gi = 309503/ug = Mm 2734/ | P | 1593.8 | P | 914.4 | P | 572.2 | P |
| | | len = 961/STRA = for | | | | | | | |
| 20 | 96704_at | Cluster Incl AF058798 Mus musculus 14-3-3 protein sigma mRNA, complete cds/cds = (163,909)/gb = AF058798/ | P | 3395 | P | 5004.1 | P | 3183.5 | P |
| | | gi = 3065926/ug = Mm 2758/len = 1430/STRA = for | | | | | | | |
| 20 | 96718_at | Cluster Incl AB012727 Mus musculus C184L-22 mRNA, complete cds/cds = UNKNOWN/gb = AB012727/gi = 6469043/ | P | 614.6 | P | 598.2 | P | 466 | P |
| | | ug = Mm 27657/len = 2532/STRA = for | | | | | | | |
| 20 | 97199_at | Cluster Incl AI854767 UI-M-BH0-aka-c-06-0-UI s1 Mus musculus cDNA, 3 end/clone = UI-M-BH0-aka-c-06-0-UI/ | P | 743.6 | P | 429.1 | P | 448.5 | P |
| | | clone_end = 3/gb = AI854767/gi = 5498673/ug = Mm 27660/len = 203/STRA = for | | | | | | | |
| 20 | 97803_at | Cluster Incl U38196 Membrane protein, palmitoylated (55 kDa)/cds = (180,1580)/gb = U38196/gi = 1616986/ug = Mm 2814/ | P | 589.8 | P | 482.5 | P | 448.4 | P |
| | | len = 1976/STRA = for | | | | | | | |
| 20 | 97875_at | Cluster Incl AW123694: UI-M-BH2.1-apl-c-08-0-UI s1 Mus musculus cDNA, 3 end/clone = UI-M-BH2 1-apl-c-08-0-UI/ | P | 512.5 | P | 492.8 | P | 197.6 | P |
| | | clone_end = 3/gb = AW123694/gi = 6099224/ug = Mm 28353/len = 479/STRA = for | | | | | | | |
| 20 | 97882_at | Cluster Incl AB032902: Mus musculus Sec61 mRNA, complete cds/cds = ( 120,1550)/gb = AB032902/gi = 6012185/ | P | 978.1 | P | 1201.5 | P | 1060 | P |
| | | ug = Mm 28375/len = 1804/STRA = rev | | | | | | | |
| 20 | 97890_at | Cluster Incl AW046181: UI-M-BH1-akw-d-06-0-UI s1 Mus musculus cDNA, 3 end/clone = UI-M-BH1-akw-d-06-0-UI/ | P | 911.6 | P | 652.7 | P | 818.8 | P |
| | | clone_end = 3/gb = AW046181/gi = 5906710/ug = Mm 28405/len = 376/STRA = for | | | | | | | |
| 20 | 97914_at | Cluster Incl D17666 Mouse gene for mitochondrial stress-70 protein (PBP74/CSA)/cds = (73,2112)/gb = D17666/gi = 903308/ | P | 1328.3 | P | 936.8 | P | 791.9 | P |
| | | ug = Mm 2849/len = 2998/STRA = for | | | | | | | |
| 20 | 98911_at | Cluster Incl AI837528: UI-M-AL0-abs-b-09-0-UI.s1 Mus musculus cDNA, 3 end/clone = UI-M-AL0-abs-b-09-0-UI/ | P | 345.5 | P | 152.8 | P | 207.1 | P |
| | | clone_end = 3/gb = AI837528/gi = 5471741/ug = Mm 28598/len = 340/STRA = for | | | | | | | |
| 20 | 94445_at | Cluster Incl AW125273: UI-M-BH2.1-aps-h-07-0-UI s1 Mus musculus cDNA, 3 end/clone = UI-M-BH2 1-aps-h-07-0-UI/ | P | 1425.9 | P | 935.3 | P | 1175.2 | P |
| | | clone_end = 3/gb = AW125273/gi = 6100803/ug = Mm 28777/len = 414/STRA = for | | | | | | | |
| 20 | 94450_at | Cluster Incl AI854202 UI-M-BH0-ajz-d-08-0-UI.s1 Mus musculus cDNA, 3 end/clone = UI-M-BH0-ajz-d-08-0-UI/ | P | 682 | P | 391.2 | P | 486.4 | P |
| | | clone_end = 3/gb = AI854202/gi = 5498108/ug = Mm 28786/len = 543/STRA = for | | | | | | | |
| 20 | 94486_at | Cluster Incl AW125178: UI-M-BH2.1-apz-g-02-0-UI s1 Mus musculus cDNA, 3 end/clone = UI-M-BH2 1-apz-g-02-0-UI/ | P | 1370.9 | P | 1379.1 | P | 926.8 | P |
| | | clone_end = 3/gb = AW125178/gi = 6100708/ug = Mm 28892/len = 249/STRA = for | | | | | | | |
| 20 | 94492_at | Cluster Incl AB025406 Mus musculus mRNA for sid23p, complete cds/cds = (34,531)/gb = AB025406/gi = 5931560/ | P | 3685.2 | P | 4280.8 | P | 3563.3 | P |
| | | ug = Mm 28919/len = 1639/STRA = for | | | | | | | |
| 20 | 95079_at | Cluster Incl M57683 Platelet derived growth factor receptor, alpha polypeptide/cds = (78,3347)/gb = M57683/gi = 199783/ | P | 961.6 | P | 94.9 | P | 417.4 | P |
| | | ug = Mm 2924/len = 3564/STRA = for | | | | | | | |
| 20 | 95102_at | Cluster Incl AW123754 UI-M-BH2.1-apm-a-08-0-UI s1 Mus musculus cDNA, 3 end/clone = UI-M-BH2.1-apm-a-08-0-UI/ | P | 977.2 | P | 805.1 | P | 961.5 | P |
| | | clone_end = 3/gb = AW123754/gi = 6099284/ug = Mm 29345/len = 395/STRA = for | | | | | | | |
| 20 | 95135_at | Cluster Incl AI844396 UI-M-AL1-ahp-e-05-0-UI s1 Mus musculus cDNA, 3 end/clone = UI-M-AL1-ahp-e-05-0-UI/ | P | 570.2 | P | 594.4 | P | 439.2 | P |
| | | clone_end = 3/gb = AI844396/gi = 5488302/ug = Mm 29429/len = 432/STRA = for | | | | | | | |
| 20 | 95657_f_at | Cluster Incl AW125164 UI-M-BH2.1-apz-e-09-0-UI s1 Mus musculus cDNA, 3 end/clone = UI-M-BH2 1-apz-e-09-0-UI/ | P | 427.4 | P | 500.9 | P | 521.1 | P |
| | | clone_end = 3/gb = AW125164/gi = 6100694/ug = Mm 29541/len = 210/STRA = for | | | | | | | |
| 20 | 95688_at | Cluster Incl Y08460: Degenerative spermatocyte homolog (Drosophila)/cds = (41,1012)/gb = Y08460/gi = 2225940/ | P | 1558.3 | P | 1158.3 | P | 1188.5 | P |
| | | ug = Mm 29648/len = 1778/STRA = for | | | | | | | |
| 20 | 96245_at | Cluster Incl AW124530: UI-M-BH2 1-apo-g-09-0-UI s1 Mus musculus cDNA, 3 end/clone = UI-M-BH2 1-apo-g-09-0-UI/ | P | 348.6 | P | 402 | P | 378.3 | P |
| | | clone_end = 3/gb = AW124530/gi = 6100060/ug = Mm 29810/len = 487/STRA = for | | | | | | | |
| 20 | 96252_at | Cluster Incl AJ005073 Mus musculus mRNA for Alix (ALG-2-Interacting protein X), complete CDS/cds = (0,2609)/ | P | 909.2 | P | 581.5 | P | 756.9 | P |
| | | gb = AJ005073/gi = 3550455/ug = Mm 29816/len = 2610/STRA = for | | | | | | | |
| 20 | 96340_at | Cluster Incl AW124185 UI-M-BH2 1-aph-a-04-0-UI s1 Mus musculus cDNA, 3 end/clone = UI-M-BH2 1-aph-a-04-0-UI/ | P | 463.6 | P | 380 | P | 356.8 | P |
| | | clone_end = 3/gb = AW124185/gi = 6099715/ug = Mm 29987/len = 399/STRA = for | | | | | | | |
| 20 | 97451_at | Cluster Incl AI837599 UI-M-AK0-adg-a-10-0-UI s1 Mus musculus cDNA, 3 end/clone = UI-M-AK0-adg-a-10-0-UI/ | P | 536.5 | P | 151.4 | P | 235.7 | P |
| | | clone_end = 3/gb = AI837599/gi = 5471812/ug = Mm 30251/len = 449/STRA = for | | | | | | | |
| 20 | 97529_at | Cluster Incl AJ002390 Annexin 8/cds = (53,1036)/gb = AJ002390/gi = 2612794/ug = Mm 3267/len = 1888/STRA = for | P | 1607.9 | P | 971.2 | P | 1380 | P |
| 20 | 98114_at | Cluster Incl AF003348 Niemann Pick type C1/cds = (123,3959)/gb = AF003348/gi = 2251241/ug = Mm 3484/len = 5029/ | P | 396.5 | P | 325.1 | P | 385.6 | P |
| | | STRA = for | | | | | | | |
| 20 | 98130_at | Cluster Incl U85089 Mus musculus thioredoxin mRNA, nuclear gene encoding mitochondrial protein, complete cds/ | P | 791.2 | P | 813.3 | P | 800.3 | P |
| | | cds = (51,551)/gb = U85089/gi = 1814410/ug = Mm 3533/len = 1182/STRA = for | | | | | | | |
| 20 | 98153_at | Cluster Incl L20509 Chaperonin subunit 3 (gamma)/cds = (83,1717)/gb = L20509/gi = 347838/ug = Mm 3576/len = 1937/ | P | 852.1 | P | 728.4 | P | 635.5 | P |
| | | STRA = for | | | | | | | |
| 20 | 98600_at | Cluster Incl U41341: Mus musculus endothelial monocyte-activating polypeptide I mRNA, complete cds/cds = (0,296)/ | P | 3157.9 | P | 3298.9 | P | 3137.9 | P |
| | | gb = U41341/gi = 1150723/ug = Mm.383/len = 331/STRA = for | | | | | | | |
| 20 | 98608_at | Cluster Incl AI845886 UI-M-AO1-aeg-b-09-0-UI.s1 Mus musculus cDNA, 3 end/clone = UI-M-AO1-aeg-h-09-0-UI/ | P | 1035 | P | 1109 | P | 1175.3 | P |
| | | clone_end = 3/gb = AI845886/gi = 5489792/ug = Mm.3845/len = 447/STRA = rev | | | | | | | |
| 20 | 98628_f_at | Cluster Incl AF003695: Hypoxia Inducible factor 1, alpha subunit/cds = (257,2767)/gb = AF003695/gi = 2197094/ | P | 1987.8 | P | 1049.3 | P | 1113.6 | P |
| | | ug = Mm 3879/len = 3973/STRA = for | | | | | | | |
| 20 | 98629_f_at | Cluster Incl Y09085 Hypoxia inducible factor 1, alpha subunit/cds = (153,2585)/gb = Y09085/gi = 2113880/ug = Mm 3879/ | P | 2031.2 | P | 1035 | P | 1256.3 | P |
| | | len = 3774/STRA = for | | | | | | | |
| 20 | 99087_at | Cluster Incl AW060179: UI-M-BH1-amu-d-09-0-UI s1 Mus musculus cDNA, 3 end/clone = UI-M-BH1-amu-d-09-0-UI/ | P | 760.1 | P | 713.5 | P | 645.4 | P |
| | | clone_end = 3/gb = AW060179/gi = 6007945/ug = Mm 38994/len = 244/STRA = rev | | | | | | | |
| 20 | 99101_at | Cluster Incl AB012580 Mus musculus mRNA for elF3 p66, complete cds/cds = (96,1739)/gb = AB012580/gi = 2992163/ | P | 849.6 | P | 1030.8 | P | 840.6 | P |
| | | ug = Mm 3955/len = 1849/STRA = for | | | | | | | |
| 20 | 99182_at | Cluster Incl AI846712: UI-M-AN1-afi-g-12-0-UI s1 Mus musculus cDNA, 3 end/clone = UI-M-AN1-afi-g-12-0-UI/ | P | 430.6 | P | 434.2 | P | 384.3 | P |
| | | clone_end = 3/gb = AI846712/gi = 5490618/ug = Mm 41801/len = 381/STRA = rev | | | | | | | |
| 20 | 100051_at | Cluster Incl U17297 Erythrocyte protein band 7 2/cds = (90,944)/gb = U17297/gi = 972906/ug = Mm.4441/len = 2821/ | P | 1057.5 | P | 844.8 | P | 1171.4 | P |
| | | STRA = for | | | | | | | |
| 20 | 100123_f_at | Cluster Incl X15202: Mouse mRNA for fibronectin receptor beta-chain (VLA5-homolog)/cds = (0,2398)/gb = X15202/ | P | 3209.1 | P | 2653.4 | P | 3124.7 | P |
| | | gi = 50986/ug = Mm 4712/len = 3491/STRA = for | | | | | | | |
| 20 | 100124_r_at | Cluster Incl X15202: Mouse mRNA for fibronectin receptor beta-chain (VLA5-homolog)/cds = (0,2398)/gb = X15202/ | P | 1392 | P | 923.9 | P | 1119.2 | P |
| | | gi = 50986/ug = Mm 4712/len = 3491/STRA = for | | | | | | | |
| 20 | 100127_at | Cluster Incl M35523 Cellular retinoic acid binding protein II/cds = (115,531)/gb = M35523/gi = 192705/ug = Mm 4757/len = 868/ | P | 247 | P | 109.2 | P | 181.4 | P |
| | | STRA = for | | | | | | | |
| 20 | 100142_at | Cluster Incl X95480: DiGeorge syndrome gene c/cds = (133,1779)/gb = X95480/gi = 1550786/ug = Mm 4891/len = 2987/ | P | 365 | P | 256.8 | P | 259.8 | P |
| | | STRA = for | | | | | | | |
| 20 | 100154_at | Cluster Incl AIB36367: UI-M-AP0-abi-b-11-0-UI s1 Mus musculus cDNA, 3 end/clone = UI-M-AP0-abi-b-11-0-UI/ | P | 504 | P | 212.5 | P | 262.6 | P |
| | | clone_end = 3/gb = AI836367/gi = 5470580/ug = Mm 4975/len = 332/STRA = for | | | | | | | |
| 20 | 100535_at | Cluster Incl U76112 Mus musculus translation repressor NAT1 mRNA, complete cds/cds = (306,3026)/gb = U76112/ | P | 480.6 | P | 480 | P | 477 | P |
| | | gi = 1903415/ug = Mm 525/len = 3789/STRA = for | | | | | | | |
| 20 | 100581_at | Cluster Incl U59807 Cystatin B/cds = (0,296)/gb = U59807/gi = 1439569/ug = Mm 6095/len = 297/STRA = for | P | 3363 | P | 2889.7 | P | 3437.9 | P |
| 20 | 100597_at | Cluster Incl AW049730: UI-M-BH1-ani-g-03-0-UI s1 Mus musculus cDNA, 3 end/clone = UI-M-BH1-ani-g-03-0-UI/ | P | 275.6 | P | 151.7 | P | 232 | P |
| | | clone_end = 3/gb = AW049730/gi = 5910259/ug = Mm 6375/len = 348/STRA = for | | | | | | | |
| 20 | 100606_at | Cluster Incl M18070 Mouse (with short incubation period) prion protein (PRNP) gene, complete cds/cds = (10,774)/ | P | 2562.9 | P | 2934.7 | P | 2927.6 | P |
| | | gb = M18070/gi = 200528/ug = Mm 648/len = 775/STRA = for | | | | | | | |
| 20 | 101025_f_at | Cluster Incl AJ005559 Small proline-rich protein 2A/cds = (0,251)/gb = AJ005559/gi = 3093357/ug = Mm 6853/len = 252/ | P | 1700.6 | P | 1482.6 | P | 609.3 | P |
| | | STRA = for | | | | | | | |
| 20 | 101482_at | Cluster Incl D85137 Mouse mRNA for PP1gamma, complete cds/cds = (107,1078)/gb = D85137/gi = 1332408/ug = Mm 7793/ | P | 1177.4 | P | 1118.7 | P | 939.5 | P |
| | | len = 2200/STRA = for | | | | | | | |
| 20 | 101487_f_at | Cluster Incl U47737 Mus musculus thymic shared antigen-1 (TSA-1) gene, complete cds/cds = (106,498)/gb = U47737/ | P | 2869.4 | P | 1632.2 | P | 1494.3 | P |
| | | gi = 1199649/ug = Mm.788/len = 1117/STRA = for | | | | | | | |
| 20 | 101963_at | Cluster Incl X06086 Cathepsin L/cds = (68,1072)/gb = X06086/gi = 53046/ug = Mm 930/len = 1374/STRA = for | P | 1817.1 | P | 1144 | P | 1575.1 | P |
| 20 | 101993_at | Cluster Incl X56304 Tenascin C/cds = (126,6185)/gb = X56304/gi = 54768/ug = Mm 980/len = 6831/STRA = for | P | 1615.8 | P | 443 | P | 786.5 | P |
| 20 | 102052_at | Cluster Incl AA871791 vq41a10 r1 Mus musculus cDNA, 5 end/clone = IMAGE-1096794/clone_end = 5/gb = AA871791/ | P | 443.8 | P | 304 | P | 406 | P |
| | | gi = 2967236/ug = Mm.10297/len = 292/STRA = for | | | | | | | |
| 20 | 102315_at | Cluster Incl AW124570 UI-M-BH2 1-apt-d-08-0-UI s1 Mus musculus cDNA, 3 end/clone = UI-M-BH2 1-apt-d-08-0-UI/ | P | 273 | P | 344.4 | P | 246.1 | P |
| | | clone_end = 3/gb = AW124570/gi = 6100100/ug = Mm 10665/len = 450/STRA = for | | | | | | | |
| 20 | 102348_at | Cluster Incl AI551087 vx36f11.x1 Mus musculus cDNA, 3 end/clone = IMAGE-1277325/clone_end = 3/gb = AI551087/ | P | 1359.2 | P | 1497 | P | 1058.7 | P |
| | | gi = 4483450/ug = Mm.11223/len = 446/STRA = rev | | | | | | | |
| 20 | 103035_at | Cluster Incl U60020: Transporter 1, ABC (ATP binding cassette)/cds = (0,2174)/gb = U60020/gi = 1399904/ug = Mm 16122/ | P | 424.8 | P | 238.9 | P | 307.5 | P |
| | | len = 2175/STRA = for | | | | | | | |
| 20 | 103059_at | Cluster Incl X93038 M musculus mRNA for MATB protein/cds = (87,353)/gb = X93038/gi = 1085067/ug = Mm 1662/len = 522/ | P | 2439.9 | P | 2185 | P | 2232.9 | P |
| | | STRA = rev | | | | | | | |
| 20 | 103416_at | Cluster Incl AI844810 UI-M-AH1-agx-b-06-0-UI s1 Mus musculus cDNA, 3 end/clone = UI-M-AH1-agx-b-06-0-UI/ | P | 1573.9 | P | 984.5 | P | 973.4 | P |
| | | clone_end = 3/gb = AI844810/gi = 5488716/ug = Mm 18856/len = 520/STRA = for | | | | | | | |
| 20 | 103589_at | Cluster Incl AF053235 Mus musculus keratin 16 mRNA, complete cds/cds = (12,1421)/gb = AF053235/gi = 3925354/ | P | 3178.4 | P | 502.5 | P | 574.3 | P |
| | | ug = Mm.19889/len = 1580/STRA = for | | | | | | | |
| 20 | 103877_at | Cluster Incl AW060485 UI-M-BH1-ann-a-09-0-UI s1 Mus musculus cDNA, 3 end/clone = UI-M-BH1-ann-a-09-0-UI/ | P | 219.9 | P | 261.1 | P | 305.7 | P |
| | | clone_end = 3/gb = AW060485/gi = 6008236/ug = Mm 21184/len = 263/STRA = for | | | | | | | |
| 20 | 104074_at | Cluster Incl AW212109 uo82e01.x1 Mus musculus cDNA, 3 end/clone = IMAGE-2649048/clone_end = 3/gb = AW212109/ | P | 312.2 | P | 285.3 | P | 283.4 | P |
| | | gi = 6518110/ug = Mm 21687/len = 298/STRA = for | | | | | | | |
| 20 | 104105_at | Cluster Incl AI854665 UI-U-BH0-akc-a-09-0-UI s1 Mus musculus cDNA, 3 end/clone = UI-M-BH0-akc-a-09-0-UI/ | P | 456.2 | P | 287.2 | P | 402.6 | P |
| | | clone_end = 3/gb = AI854665/gi = 5498571/ug = Mm 21892/len = 486/STRA = for | | | | | | | |
| 20 | 104283_at | Cluster Incl AI037493 ub62a05 r1 Mus musculus cDNA, 5 end/clone = IMAGE-1382288/clone_end = 5/gb = AI037493 | P | 131.3 | P | 83.6 | P | 114.6 | P |
| | | gi = 3261184/ug = Mm 22252/len = 416/STRA = for | | | | | | | |
| 20 | 104300_at | Cluster Incl AI117936 uc41g02 r1 Mus musculus cDNA, 5 end/clone = IMAGE-1400594/clone_end = 5/gb = AI117936/ | P | 880 | P | 966.1 | P | 626.9 | P |
| | | gi = 3518260/ug = Mm 22343/len = 182/STRA = for | | | | | | | |
| 20 | 104369_at | Cluster Incl AB012033 Mus musculus gene for keratin 6 alpha/cds = (0,1661)/gb = AB012033/gi = 3882140/ug = Mm 22629/ | P | 2284.3 | P | 2842.4 | P | 1488.5 | P |
| | | len = 2259/STRA = for | | | | | | | |
| 20 | 104370_s_at | Cluster Incl K02108 Keratin complex 2, gene 6A/cds = (49,1710)/gb = K02108/gi = 198634/ug = Mm 22629/len = 2005/ | P | 2772.4 | P | 1061.6 | P | 496.1 | P |
| | | STRA = for | | | | | | | |
| 20 | 104386_f_at | Clusler Incl AI843901: UI-M-AK1-aeu-g-04-0-UI s1 Mus musculus cDNA, 3 end/clone = UI-M-AK1-aeu-g-04-0-UI/ | P | 1308.8 | P | 1082.7 | P | 1086.5 | P |
| | | clone_end = 3/gb = AI843901/gi = 5478114/ug = Mm 227/len = 300/STRA = for | | | | | | | |
| 20 | 104579_r_at | Cluster Incl AI195392: ue72102.r1 Mus musculus cDNA, 5 end/clone = IMAGE-149666/clone_end = 5/gb = AI195392/ | P | 471 | P | 497 | P | 452.7 | P |
| | | gi = 3747998/ug = Mm.23961/len = 262/STRA = for | | | | | | | |
| 20 | 104605_at | Cluster Incl AW047554 UI-M-BH1-ama-g-03-0-UI s1 Mus musculus cDNA, 3 end/clone = UI-M-BH1-ama-g-03-0-UI/ | P | 837.3 | P | 933.9 | P | 905.7 | P |
| | | clone_end = 3/gb = AW047554/gi = 5908083/ug = Mm 24126/len = 312/STRA = for | | | | | | | |
| 20 | 104628_at | Cluster Incl X61172.Mouse mRNA for alpha-mannosidase II/cds = (87,3539)/gb = X61172/gi = 49943/ug = Mm 2433/ | P | 617.9 | P | 544.4 | P | 392.2 | P |
| | | len = 6080/STRA = for | | | | | | | |
| 20 | 104716_at | Cluster Incl X60367: Retinol binding protein 1, cellular/cds = UNKNOWN/gb = X60367/gi = 50547/ug = Mm 2450/len = 2633/ | P | 690.6 | P | 274.6 | P | 267 | P |
| | | STRA = for | | | | | | | |
| 20 | 94367_at | Cluster Incl AI850362 UI-M-BG1-aig-b-06-0-UI s1 Mus musculus cDNA, 3 end/clone = UI-M-BG1-aig-b-06-0-UI/ | P | 563.9 | P | 574.7 | P | 476.7 | P |
| | | clone_end = 3/gb = AI850362/gi = 5494268/ug = Mm 25309/len = 494/STRA = for | | | | | | | |
| 20 | 94391_at | Cluster Incl AW123650 UI-M-BH2 1-apl-g-08-0-U1.s1 Mus musculus cDNA, 3 end/clone = UI-M-BH2 1-apl-g-08-0-UI/ | P | 199.1 | P | 86.8 | P | 95.6 | P |
| | | clone_end = 3/gb = AW123650/gi = 6099180/ug = Mm 25652/len = 417/STRA = for | | | | | | | |
| 20 | 94433_at | Cluster Incl AW060684 UI-M-BH1-anl-e-02-0-UI s1 Mus musculus cDNA, 3 end/clone = UI-M-BH1-anl-e-02-0-UI/ | P | 1999.3 | P | 1124.5 | P | 1339.8 | P |
| | | clone_end = 3/gb = AW060684/gi = 6008435/ug = Mm.26492/len = 383/STRA = for | | | | | | | |
| 20 | 94928_at | Cluster Incl X87128 M musculus p75 TNF receptor DNA/cds = (67,133)/gb = X87128/gi = 809043/ug = Mm 2666/len = 134/ | P | 155.2 | P | 110.1 | P | 41 | P |
| | | STRA = for | | | | | | | |
| 20 | 94991_at | Cluster Incl AW046661.UI-M-BH1-ald-g-02-0-UI s1 Mus musculus cDNA, 3 end/clone = U-M-BH1-ald-g-02-0-UI/ | P | 205.6 | P | 164.7 | P | 156 | P |
| | | clone_end = 3/gb = AW046661/gi = 5907190/ug = Mm 27313/len = 430/STRA = for | | | | | | | |
| 20 | 95542_at | Cluster Incl AI835858.UI-M-AI0-aat-c-09-0-UI s1 Mus musculus cDNA, 3 end/clone = UI-M-AI0-aal-c-09-0-UI/clone_end = 3/ | P | 1903.7 | P | 1576.8 | P | 1277.5 | P |
| | | gb = AI835858/gi = 5470071/ug = Mm 27685/len = 469/STRA = rev | | | | | | | |
| 20 | 96187_at | Cluster Incl AI837021 UI-M-AP0-abj-a-11-0-UI s2 Mus musculus cDNA, 3 end/clone = UI-M-AP0-abj-a-11-0-UI/ | P | 709 | P | 477.6 | P | 532.7 | P |
| | | clone_end = 3/gb = AI837021/gi = 5471234/ug = Mm 28488/len = 182/STRA = for | | | | | | | |
| 20 | 97333_at | Cluster Incl AW121185 UI-M-BH2 3-aok-b-01-0-UI s1 Mus musculus cDNA, 3 end/clone = UI-M-BH2 3-aok-b-01-0-UI/ | P | 1048.5 | P | 1210.7 | P | 1162.7 | P |
| | | clone_end = 3/gb = AW121185/gi = 6096518/ug = Mm 29547/len = 415/STRA = for | | | | | | | |
| 20 | 97383_at | Cluster Incl AI848623 UI-M-AH1-agv-d-05-0-UI s1 Mus musculus cDNA, 3 end/clone = UI-M-AH1-agv-d-05-0-UI/ | P | 1062.7 | P | 517.9 | P | 472.5 | P |
| | | clone_end = 3/gb = AI848623/gi = 5492569/ug = Mm 29762/len = 466/STRA = rev | | | | | | | |
| 20 | 97419_at | Cluster Incl AW060956 UI-M-BH1-anv-g-02-0-UI s1 Mus musculus cDNA, 3 end/clone = UI-M-BH1-anv-g-02-0-UI/ | P | 258.5 | P | 235.7 | P | 260.6 | P |
| | | clone_end = 3/gb = AW060956/gi = 6008707/ug = Mm 29985/len = 493/STRA = for | | | | | | | |
| 20 | 97930= f = at | Cluster Incl AF033620 CD151 antigen/cds = (82,843)/gb AF033620/gi = 3298592/ug = Mm 30246/len = 1618/STRA = for | P | 815.6 | P | 899.6 | P | 913 | P |
| 20 | 97932_f_at | Cluster Incl D89290 CD151 antigen/cds = (327,1088)/gb = D89290/gi = 2447006/ug = Mm 30246/len = 1819/STRA = for | P | 1165.6 | P | 1495.1 | P | 1352 | P |
| 20 | 97950_at | Cluster Incl X75129 Xanthine dehydrogenase/cds = (25,4032)/gb = X75129/gi = 473040/ug = Mm 3043/len = 4554/STRA = for | P | 287.9 | P | 110.3 | P | 300.5 | P |
| 20 | 98001_at | Cluster Incl U58203 Mus musculus Lsc (lsc) oncogene mRNA, complete cds/cds = (91,2850)/gb = U58203/gi = 1389755/ | P | 177.7 | P | 136.3 | P | 119.5 | P |
| | | ug = Mm 3181/len = 3200/STRA = for | | | | | | | |
| 20 | 98402_at | Cluster Incl AI843799 UI-M-AN1-afc-e-09-0-UI s1 Mus musculus cDNA, 3 end/clone = UI-M-AN1-afc-e-09-0-UI/ | P | 962.4 | P | 927.6 | P | 954.7 | P |
| | | clone_end = 3/gb = AI843799/gi = 5478012/ug = Mm 3350/len = 402/STRA = for | | | | | | | |
| 20 | 98418_at | Cluster Incl U10115-Dishevelled, dsh homolog (Drosophila)/cds = (262,2349)/gb = U10115/gi = 497689/ug = Mm 3400/ | P | 564.8 | P | 412.5 | P | 359.5 | P |
| | | len = 3286/STRA = for | | | | | | | |
| 20 | 98423_at | Cluster Incl M81445 Gap junction membrane channel protein beta 2/cds = (0,680)/gb = M81445/gi = 192844/ug = Mm 34118/ | P | 2588.7 | P | 3793.5 | P | 2062.6 | P |
| | | len = 681/STRA = for | | | | | | | |
| 20 | 99057_at | Cluster Incl M12379 Mouse Thy-1 2 glycoprotein gene, complete cds/cds = (0,488)/gb = M12379/gi = 202040/ug = Mm 3951/ | P | 1055.1 | P | 893.2 | P | 764.2 | P |
| | | len = 489/STRA = for | | | | | | | |
| 20 | 100428_at | Cluster Incl U43327: Laminin, gamma 2/cds = (39,3617)/gb = U43327/gi = 1151216/ug = Mm 4717/len = 5159/STRA = for | P | 1302.5 | P | 883 | P | 1349.4 | P |
| 20 | 100445_f_at | Cluster Incl X91825 M musculus mRNA for SPRR1b protein/cds = (45,506)/gb = X91825/gi = 1213026/ug = Mm 4826/ | P | 4111.7 | P | 492.8 | P | 881.5 | P |
| | | len = 688/STRA = for | | | | | | | |
| 20 | 100472_at | Cluster Incl D10727: Mus musculus mRNA for NDPP-1 protein, complete cds/cds = (857,2026)/gb = D10727/gi = 220499/ | P | 494.6 | P | 442 | P | 530.9 | P |
| | | ug = Mm 4941/len = 4308/STRA = rev | | | | | | | |
| 20 | 100499_at | Cluster Incl D29797 Mouse mRNA for syntaxin 3A, complete cds/cds = (103,972)/gb = D29797/gi = 924267/ug = Mm 5122/ | P | 231.8 | P | 279.3 | P | 210.3 | P |
| | | len = 1073/STRA = for | | | | | | | |
| 20 | 100946_at | Cluster Incl AF109906 Mus musculus MHC class III region RD gene, partial cds, Bf, C2, G9A, NG22, G9, HSP70, HSP70, | P | 2174.1 | P | 1251.2 | P | 1215.4 | P |
| | | HSC701, and smRNP genes, complete cds, G7A gene, partial cds, and unknown genes/cds = (0,1928)/gb = AF109906/ | | | | | | | |
| | | gi = 3986763/ug = Mm 6388/len = 1929/STRA = for | | | | | | | |
| 20 | 102203_at | Cluster Incl M95545 Placental protein 11 related/cds = (9,1373)/gb = M95545/gi = 201907/ug = Mm.1001/len = 2407/ | P | 645.6 | P | 411.1 | P | 453.9 | P |
| | | STRA = rev | | | | | | | |
| 20 | 102248_f_at | Cluster Incl Y17138.Calcium/calmodulin-dependent serine protein kinase/cds = (492,3266)/gb = Y17138/gi = 3087817/ | P | 433.8 | P | 388.9 | P | 363.2 | P |
| | | ug = Mm.10735/len = 4393/STRA = for | | | | | | | |
| 20 | 102255_at | Cluster Incl AB015978 Oncostatin receptor/cds = (779,3691)/gb = AB015978/gi = 3721859/ug = Mm 10760/len = 4026/ | P | 260.7 | P | 75.3 | P | 96.4 | P |
| | | STRA = for | | | | | | | |
| 20 | 103217_at | Cluster Incl Y14041: Mus musculus mRNA for CASH alpha protein/cds = (74,1528)/gb = Y14041/gi = 2653419/ug = Mm.1993/ | P | 354.5 | P | 459.1 | P | 378.6 | P |
| | | len = 2770/STRA = for | | | | | | | |
| 20 | 103305_at | Cluster Incl L04678 Integrin beta 4/cds = UNKNOWN/gb = L04678/gi = 192151/ug = Mm.21117/len = 5907/STRA = for | P | 294.8 | P | 307.4 | P | 222.7 | P |
| 20 | 103306_at | Cluster Incl AW259940: um80f09 y1 Mus musculus cDNA, 5 end/clone = IMAGE-2317673/clone_end = 5/gb = AW259940/ | P | 105.5 | P | 42.2 | P | 101.1 | P |
| | | gi = 6632921/ug = Mm 21134/len = 429/STRA = for | | | | | | | |
| 20 | 103448_at | Cluster Incl M83218 Mus musculus intracellular calcium-binding protein (MRP8) mRNA, complete cds/cds = (17,286)/ | P | 3300.4 | P | 255.7 | P | 285.3 | P |
| | | gb = M83218/gi = 199809/ug = Mm 21567/len = 343/STRA = for | | | | | | | |
| 20 | 103723_at | Cluster Incl AA608387 vo42b10 r1 Mus musculus cDNA, 5 end/clone = IMAGE-1052539/clone_end = 5/gb = AA608387/ | P | 247.5 | P | 53.9 | P | 83.7 | P |
| | | gi = 2455274/ug = Mm 24208/len = 390/STRA = for | | | | | | | |
| 20 | 103930_at | Cluster lncl AI413179 mc22e12.x1 Mus musculus cDNA, 3 end/clone = IMAGE-349294/clone_end = 3/gb = AI413179/ | P | 456.4 | P | 320.6 | P | 254.4 | P |
| | | gi = 4256683/ug = Mm 25117/len = 446/STRA = for | | | | | | | |
| 20 | 104260_at | Cluster Incl AW123061: UI-M-BH2.1-apc-a-11-0-UI.s1 Mus musculus cDNA, 3 end/clone = UI-M-BH2.1-apc-a-11-0-UI/ | P | 248.5 | P | 162 | P | 195.3 | P |
| | | clone_end = 3/gb = AW123061/gi = 6098591/ug = Mm.28411/len = 288/STRA = for | | | | | | | |
| 20 | 104406_at | Cluster Incl AI060798 ub44a06 r1 Mus musculus cDNA, 5 end/clone = IMAGE-1380582/clone_end = 5/gb = AI060798/ | P | 311.6 | P | 277.7 | P | 260.5 | P |
| | | gi = 3336221/ug = Mm 28768/len = 445/STRA = for | | | | | | | |
| 20 | 104469_at | Cluster Incl M73748 Mouse OTS-8 mRNA, complete cds/cds = (166,780)/gb = M73748/gi = 200164/ug = Mm 2976/len = 1661/ | P | 260.2 | P | 228.5 | P | 253.2 | P |
| | | STRA = for | | | | | | | |
| 20 | 92978_s_at | Cluster Incl X16490 Plasminogen activator inhibitor, type II/cds = (0, 1247)/gb = X16490/gi = 53589/ug = Mm.5019/len = 1905/ | P | 967.4 | P | 206.8 | P | 474.6 | P |
| | | STRA = for | | | | | | | |
| 20 | 93371_at | Cluster Incl U88064: Mus musculus basonuclin mRNA, complete cds/cds = (43,2928)/gb = U88064/gi = 2408218/ | P | 308.3 | P | 424.2 | P | 265.9 | P |
| | | ug = Mm.6110/len = 4441/STRA = for | | | | | | | |
| 20 | 93424_at | Cluster Incl AI836915 UI-M-AJ0-aav-c-11-0-UI.s1 Mus musculus cDNA, 3 end/clone = UI-M-AJ0-aav-c-11-0-UI/ | P | 689.9 | P | 615.9 | P | 540.2 | P |
| | | clone_end = 3/gb = AI836915/gi = 5471128/ug = Mm 6478/len = 422/STRA = rev | | | | | | | |
| 20 | 94121_at | Cluster Incl AJ005566 Small proline-rich protein 2H/cds(31,357)/gb = AJ005566/gi = 3093370/ug = Mm.10693/len = 728/ | P | 1741.4 | P | 459.1 | P | 940.2 | P |
| | | STRA = rev | | | | | | | |
| 20 | 94796_at | Cluster Incl AA710643 vt51d05 r1 Mus musculus cDNA, 5 end/clone = IMAGE-1166601/clone_end = 5/gb = AA710643/ | P | 1306.2 | P | 1574 | P | 1228.6 | P |
| | | gi = 2720561/ug = Mm.1800/len = 254/STRA = for | | | | | | | |
| 20 | 100772_g_at | Cluster Incl AF068182 Mus musculus B cell linker protein BLNK mRNA, complete cds/cds = (132,1505)/gb = AF068182/ | P | 203 | P | 166.7 | P | 228.4 | P |
| | | gi = 3406752/ug = Mm.9749/len = 1719/STRA = for | | | | | | | |
| 20 | 101717_at | Cluster Incl U26176 Somatostatin receptor 4/cds = (0,1154)/gb = U26176/gi = 833840/ug = Mm 57026/len = 1155/STRA = for | P | 388.4 | P | 574.2 | P | 368 | P |
| 20 | 99701_f_at | Cluster Incl AJ005560 Small proline-rich protein 2B/cds = (0, 296)/gb = AJ005560/gi = 3093359/ug = Mm 23796/len = 297/ | P | 2415.8 | P | 990.1 | P | 861.4 | P |
| | | STRA = for | | | | | | | |
| 20 | 101621_at | Cluster Incl AF008595.Mus musculus lacrimal gland protein mRNA, complete cds/cds = (17,298)/gb = AF008595/ | P | 394.6 | P | 190.7 | P | 373.2 | P |
| | | gi = 2338287/ug = Mm 6205/len = 420/STRA = for | | | | | | | |
| 20 | 102108_f_at | Cluster Incl AI505453 vr15c02.x1 Mus musculus cDNA, 3 end/clone = IMAGE-1120706/clone_end = 3/gb = AI505453/ | P | 590.5 | P | 829.5 | P | 602.6 | P |
| | | gi = 4403304/ug = Mm 42551/len = 260/STRA = rev | | | | | | | |
| 20 | 92618_at | Cluster Incl AW125253 UI-M-BH2.1-aps-f-11-0-UI s1 Mus musculus cDNA, 3 end/clone = UI-M-BH2 1-aps-f-11-0-UI/ | P | 343.5 | P | 269.5 | P | 495.3 | P |
| | | clone_end = 3/gb = AW125253/gi = 6100783/ug = Mm.1106/len = 245/STRA = for | | | | | | | |
| 20 | 93798_at | Cluster Incl AI839988 UI-M-AH0-acs-e-11-0-UI s1 Mus musculus cDNA, 3 end/clone = UI-M-AH0-acs-e-11-0-UI/ | P | 740.1 | P | 607.5 | P | 610.9 | P |
| | | clone_end = 3/gb = AI839988/gi = 5474201/ug = Mm.2140/len = 352/STRA = for | | | | | | | |
| 20 | 96957_at | Cluster Incl AB006463 Mus musculus mRNA for KIP, complete cds/cds = (26,601)/gb = AB006463/gi = 4185596/ | P | 418.7 | P | 401 | P | 276.1 | A |
| | | ug = Mm.30217/len = 687/STRA = for | | | | | | | |
| 20 | 99081_at | Cluster Incl AF010254.Complement component 1 inhibitor/cds = (66, 1580)/gb = AF010254/gi = 3236325/ug = Mm 38888/ | P | 548.2 | P | 358.5 | P | 205.8 | P |
| | | len = 1775/STRA = for | | | | | | | |
| 20 | 99159_at | Cluster Incl AI842675 UI-M-AO1-aeo-b-01-0-UI s1 Mus musculus cDNA, 3 end/clone = UI-M-AO1-aeo-b-01-0-UI/ | P | 393.1 | P | 206.1 | P | 232.4 | A |
| | | clone_end = 3/gb = AI842675/gi = 5476888/ug = Mm.41656/len = 280/STRA = for | | | | | | | |
| 20 | 100610_at | Cluster Incl AF058298 Mus musculus calpain small subunit mRNA, complete cds/cds = (34,843)/gb = AF058298/ | P | 930.3 | P | 838.2 | P | 704.1 | P |
| | | gi = 3511115/ug = Mm 6534/len = 1401/STRA = for | | | | | | | |
| 20 | 101024_i_at | Cluster Incl AJ005559 Small proline-rich protein 2A/cds = (0,251)/gb = AJ005559/gi = 3093357/ug = Mm.6853/len = 252/ | P | 1917.6 | P | 726 | P | −216.7 | A |
| | | STRA = for | | | | | | | |
| 20 | 95035_at | Cluster Incl AI467390. vd15c12 x1 Mus musculus cDNA, 3 end/clone = IMAGE-792598/clone_end = 3/gb = AI467390/ | P | 418.6 | P | 418.6 | A | 281.5 | P |
| | | gi = 4320727/ug = Mm.27546/len = 326/STRA = for | | | | | | | |
| 20 | 96207_at | Cluster Incl AW046449 UI-M-BH1-akp-e-11-0-UI s1 Mus musculus cDNA, 3 end/clone = UI-M-BH1-akp-e-11-0-UI/ | P | 688.7 | P | 679.9 | P | 604.7 | P |
| | | clone_end = 3/gb = AW046449/gi = 5906978/ug = Mm 28645/len = 373/STRA = for | | | | | | | |
| 20 | 96212_at | Cluster Incl AI853918 UI-M-BH0-aju-f-03-0-UI.s1 Mus musculus cDNA, 3 end/clone = UI-M-BH0-aju-f-03-0-UI/ | P | 275.1 | P | 303.5 | P | 250.4 | A |
| | | clone_end = 3/gb = AI853918/gi = 5497824/ug = Mm.28662/len = 352/STRA = for | | | | | | | |
| 20 | 96738_at | Cluster Incl U41765: Mus musculus metalloprotease/disintegrin/cysteine rich protein precursor (MDC9) cDNA, complete cds/ | P | 334.8 | P | 215.1 | P | 221.4 | P |
| | | cds = (13,2550)/gb = U41765/gi = 1235675/ug = Mm.28908/len = 3850/STRA = for | | | | | | | |
| 20 | 98018_at | Cluster Incl L39017 Protein C receptor, endothelial/cds = (55, 783)/gb = L39017/gi = 728480/ug = Mm 3243/len = 1358/ | P | 132.6 | P | 162.4 | P | 81.4 | P |
| | | STRA = for | | | | | | | |
| 20 | 98427_s_at | Cluster Incl M57999.Nuclear factor of kappa light chain gene enhancer in B-cells 1, p105/cds = (291,3206)/gb = M57999/ | P | 352.3 | P | 215.5 | P | 193.4 | A |
| | | gi = 201931/ug = Mm.3420/len = 3892/STRA = for | | | | | | | |
| 20 | 101902_at | Cluster Incl X17459 Recombining binding protein suppressor of hairless (Drosophila)/cds = (0,1580)/gb = X17459/gi = 52756/ | P | 159.4 | P | 78.1 | A | 90.7 | P |
| | | ug = Mm 89726/len = 3986/STRA = for | | | | | | | |
| 20 | 103286_at | Cluster Incl AB012611 Mus musculus Hbp mRNA for Hrs binding Protein, complete cds/cds = (64,1635)/gb = AB012611/ | P | 181.5 | P | 145.6 | M | 160.8 | P |
| | | gi = 3721584/ug = Mm 20891/len = 1780/STRA = for | | | | | | | |
| 20 | 103497_at | Cluster Incl AA592351 Vo27b01 r1 Mus musculus cDNA, 5 end/ctone-IMAGE-1051081/clone_end = 5/gb = AA592351/ | P | 69.2 | P | 97.9 | P | 57 | P |
| | | gi = 2406014/ug = Mm.22318/len = 131/STRA = for | | | | | | | |
| 20 | 103991_at | Cluster Incl M61704: Mouse embryonic alkaline phosphatase gene, complete cds/cds = (0, 1589)/gb = M61704/gi = 192976/ | P | 94.2 | P | 124.4 | M | 164.7 | P |
| | | ug = Mm.26096/len = 1590/STRA = for | | | | | | | |
| 20 | 103995_at | Cluster Incl AF065441: Mus musculus FGF binding protein 1 mRNA complete cds/cds = (0,755)/gb = AF065441/ | P | 199.7 | P | 320 | P | 321.4 | P |
| | | gi = 3153884/ug = Mm.2643/len = 756/STRA = for | | | | | | | |
| 20 | 104184_at | Cluster Incl D16497: Natnuretic peptide precursor type B/cds = (78, 443)/gb = D16497/gi = 493659/ug = Mm 2740/len = 668/ | P | 57.4 | P | 58.3 | P | 39.1 | A |
| | | STRA = for | | | | | | | |
| 20 | 92369_at | Cluster Incl M92420 Transforming growth factor alpha/cds = (5, 484)/gb = M92420/gi = 202155/ug = Mm 3992/len = 497/ | P | 112.1 | P | 98.6 | P | 138.4 | P |
| | | STRA = for | | | | | | | |
| 20 | 92474_at | Cluster Incl AF083497.untilled/cds = (31, 3255)/gb = AF083497/gi = 3986489/ug = Mm.42128/len = 3924/STRA = for | P | 125.1 | P | 77.6 | P | 107 | P |
| 20 | 95984_at | Cluster Incl AI586160 vu95c12.x1 Mus musculus cDNA, 3 end/clone = IMAGE-1209910/clone_end = 3/gb = AI536160/ | P | 184 | P | 121 | P | 92 | P |
| | | gi = Mm.25035/len = 483/STRA = rev | | | | | | | |
| 20 | 96997_at | Custer Incl Y19035-Mus musculus mRNA for amilonde-sensitive Na + channel (BLINaC gene)/cds = (28, 1515)/gb = / | P | 84.2 | P | 65.5 | P | 47.7 | P |
| | | gi = 5824009/ug = Mm 88839/len = 1656/STRA = for | | | | | | | |
| 20 | 100723_f_at | Cluster Incl AJ005563: Small proline-rich protein 2E/cds = (0,230)/gb = AJ005563/gi = 3093364/ug = Mm.19358/len = 231/ | P | 1293.2 | P | 811.1 | A | 1368.3 | P |
| | | STRA = for | | | | | | | |
| 20 | 93272_at | Cluster Incl AF062076 Mus musculus palmitylated serine/threonine kinase (Psk) mRNA, complete cds/cds = (0,917)/ | P | 192.9 | P | 146.4 | P | 223.2 | A |
| | | gb = AF062076/gi = 4106341/ug = Mm.17461/len = 918/STRA = for | | | | | | | |
| 20 | 94224_s_at | Cluster Incl M74123 Mus musculus (strain C57BI/6) mRNA sequence/cds = UNKNOWN/gb = M74123/gi = 198867/ | P | 559.5 | P | −36.3 | A | 10.4 | P |
| | | ug = Mm 22262/len = 1623/STRA = for | | | | | | | |
| 20 | 94264_at | Cluster Incl AW122170 UI-M-BH2 2-aor-g-12-0-UI s1 Mus musculus cDNA, 3 end/clone = UI-M-BH2 2-aor-g-12-0-UI/ | P | 699.2 | P | 623.8 | P | 503.8 | A |
| | | clone_end = 3/gb = AW122170/gi = 6097613/ug = Mm 22467/len = 188/STRA = for | | | | | | | |
| 20 | 94918_at | Cluster Incl AI839392 UI-M-AN0-aco-b-02-0-UI.s1 Mus musculus cDNA, 3 end/clone = UI-M-AN0-aco-b-02-0-UI/ | P | 307.9 | P | 233.7 | P | 322 | M |
| | | clone_end = 3/gb = AI839392/gi = 5473605/ug = Mm 24174/len = 181/STRA = for | | | | | | | |
| 20 | 96925_at | Cluster Incl AW122429 UI-M-BH2.2-aow-g-07-0-UI.s1 Mus musculus cDNA, 3 end/clone = UI-M-BH2 2-aow-g-07-0-UI/ | P | 91.4 | P | 94.6 | P | −25.6 | A |
| | | clone_end = 3/gb = AW122429/gi = 6097940/ug = Mm.30161/len = 477/STRA = for | | | | | | | |
| 20 | 101551_s_at | Cluster Incl X78989 M. musculus mRNA for testin/cds = (120,1226)/gb = X78989/gi = 475207/ug = Mm.88645/len = 1377/ | P | 110.1 | P | 169.1 | P | 132 | A |
| | | STRA = for | | | | | | | |
| 20 | 94380_at | Cluster Incl AI852581 UI-M-BH0-aiu-a-11-0-UI s1 Mus musculus cDNA, 3 end/clone = UI-M-BH0-aiu-a-11-0-UI/ | P | 712.5 | P | 298.5 | P | 560.2 | P |
| | | clone_end = 3/gb = AI852581/gi = 5496487/ug = Mm 25576/len = 399/STRA = for | | | | | | | |
| 20 | 95591_at | Cluster Incl AI843335 UI-M-AQ1-adz-b-02-0-UI s1 Mus musculus cDNA, 3 end/clone = UI-M-AQ1-adz-b-02-0-UI/ | P | 293.8 | P | 369.8 | P | 349.7 | M |
| | | clone_end = 3/gb = AI843335/gi = 5477548/ug = Mm.27895/len = 197/STRA = for | | | | | | | |
| 20 | 95599_at | Cluster Incl D28941 Mouse mRNA for alpha-2,3-sialyltransferase, complete cds/cds = (128,1129)/gb = D28941/gi = 469215/ | P | 237.1 | P | 282.2 | P | 180.5 | M |
| | | ug = Mm 2793/len = 1919/STRA = for | | | | | | | |
| 20 | 96208_at | Cluster Incl AW123514.UI-M-BH2 1-aqb-c-04-0-UI s1 Mus musculus cDNA, 3 end/clone = UI-M-BH2 1-aqb-c-04-0-UI/ | P | 198.9 | P | 197.2 | P | 223.2 | A |
| | | clone_end = 3/gb = AW123514/gi = 6099044/ug = Mm 28646/len = 374/STRA = for | | | | | | | |
| 20 | 96824_at | Cluster Incl AB025354: Mus musculus genes for Supl15h and Sox15.partial cds/cds = (0,168)/gb = AB025354/gi = 5103143/ | P | 499.8 | P | 604.4 | P | 572.1 | A |
| | | ug = Mm 29387/len = 169/STRA = for | | | | | | | |
| 20 | 97354_at | Cluster Incl AI836082: UI-M-AJ0-abb-f-05-0-UI s1 Mus musculus cDNA, 3 end/clone = UI-M-AJ0-abb-f-05-0-UI/ | P | 261.4 | P | 270.3 | P | 117.9 | A |
| | | clone_end = 3/gb = AI836082/gi = 5470295/ug = Mm 29630/len = 432/STRA = rev | | | | | | | |
| 20 | 99489_at | Cluster Incl U23921 Mus musculus osmotic stress protein 94 (Osp94) mRNA, complete cds/cds = (182,2698)/gb = U23921/ | P | 169.2 | P | 174.9 | P | 179.4 | A |
| | | gi = 1098540/ug = Mm 4150/len = 2898/STRA = for | | | | | | | |
| 20 | 99952_at | Cluster Incl X93035 Glycoprotein 39/cds = (65,1210)/gb = X93035/gi = 1085065/ug = Mm 4376/len = 1616/STRA = rev | P | 262.9 | P | 60.2 | P | 92.5 | P |
| 20 | 100882_at | Cluster Incl AF003525 Defensin beta 1/cds = (42,251)/gb = AF003525/gi = 2197074/ug = Mm.5341/len = 398/STRA = for | P | 250.9 | P | 118.1 | M | 169.9 | A |
| 20 | 103222_at | Cluster Incl L21671 Mouse Eps8 mRNA sequence, complete cds/cds = (245,2710)/gb = L21671/gi = 309216/ug = Mm 2012/ | P | 60.8 | P | 73.9 | P | 53.3 | A |
| | | len = 3550/STRA = rev | | | | | | | |
| 20 | 104173_at | Cluster Incl AA797989 ub60d04.r1 Mus musculus cDNA, 5 end/clone = IMAGE-1382119/clone_end = 5/gb = AA797989/ | P | 291.6 | M | 357.5 | P | 327.3 | P |
| | | gi = 2860944/ug = Mm 27253/len = 413/STRA = for | | | | | | | |
| 20 | 104211_at | Cluster Incl D13867 Mouse mRNA for VLA-3 alpha subunit/cds = (0, 3161)/gb = D13867/gi = 220634/ug = Mm 2775/len = 3162/ | P | 306.8 | P | 142.1 | A | 300.6 | A |
| | | STRA = for | | | | | | | |
| 20 | 92979_at | Cluster Incl X63190 Polyomavirus enhancer activator 3/cds = (125, 1792)/gb = X63190/gi = 53627/ug = Mm 5025/len = 2410/ | P | 218.7 | P | 232.3 | P | 214.5 | A |
| | | STRA = for | | | | | | | |
| 20 | 93672_at | Cluster Incl U09928 Protein kinase, interferon inducible double stranded RNA dependent/cds = (16,1563)/gb = U09928/ | P | 186.2 | P | 99.2 | A | 110.3 | P |
| | | gi = 3273413/ug = Mm 807/len = 2191/STRA = for | | | | | | | |
| 20 | 93914_at | Cluster Incl M20658 Interleukin 1 receptor, type 1/cds = (224,1954)/gb = M20658/gi = 198300/ug = Mm 896/len = 4740/ | P | 286 | P | 208.6 | A | 123.3 | M |
| | | STRA = for | | | | | | | |
| 20 | 94753_at | Clusler Incl M80632 Guanine nucleotide binding protein, alpha 15/cds = (42,1166)/gb = M80632/gi = 193570/ug = Mm 1546/ | P | 140.5 | P | 159.8 | P | 36.1 | P |
| | | len = 1353/STRA = for | | | | | | | |
| 20 | 100353_g_at | Cluster Incl AA919208 vy50h03 r1 Mus musculus cDNA, 5 end/clone = IMAGE-1298933/clone_end = 5/gb = AA919208/ | P | 298.9 | P | 301.4 | P | −6.3 | A |
| | | gi = 3065987/ug = Mm 88724/len = 501/STRA = for | | | | | | | |
| 20 | 95794_f_at | Cluster Incl AJ005567 Small proline-rich protein 21/cds = (0,230)/gb = AJ005567/gi = 3093372/ug = Mm 87821/len = 231/ | P | 458.1 | P | −136.5 | A | 599.8 | P |
| | | STRA = for | | | | | | | |
| 20 | 93602_at | Cluster Incl AF074714 Mus musculus milogen-and stress-activated protein kinase-2 (mMSK2) mRNA, complete cds/ | P | 811.4 | P | 525.2 | A | 408.3 | A |
| | | cds = (52,2373)/gb = AF074714/gi = 3786405/ug = Mm 20914/len = 3120/STRA = for | | | | | | | |
| 20 | 96259_at | Cluster Incl U35646 Puromycin-sensitive aminopeptidase/cds = (105,2867)/gb = U35646/gi = 1184160/ug = Mm.29824/ | P | 554.7 | P | 466.7 | P | 541.3 | P |
| | | len = 3051/STRA = for | | | | | | | |
| 20 | 100621_at | Cluster Incl AI848825 UI-M-AJ1-ahb-b-03-0-UI s1 Mus musculus cDNA, 3 end/clone = UI-M-AJ1-ahb-b-03-0-UI/ | P | 878.6 | P | 255.8 | A | 3.2 | A |
| | | clone_end = 3/gb = AI848825/gi = 5492731/ug = Mm.6588/len = 342/STRA = for | | | | | | | |
| 20 | 104136_at | Cluster Incl AI840413 UI-M-AH0-acv-h-08-0-UI.s1 Mus musculus cDNA, 3 end/clone = UI-M-AH0-acv-h-08-0-UI/ | P | 165.8 | P | 154.4 | P | 175.6 | A |
| | | clone_end = 3/gb = AI840413/gi = 5474626/ug = Mm.22094/len = 266/STRA = rev | | | | | | | |
| 20 | 104374_at | Cluster Incl M64086 Mouse spi2 proteinase inhibitor (spi2/eb4) mRNA, complete cds/cds = (60,1316)/gb = M64086/ | P | 1076.7 | P | 84.2 | P | 102.9 | M |
| | | gi = 201039/ug = Mm.22650/len = 2035/STRA = rev | | | | | | | |
| 20 | 104737_at | Cluster Incl AA689670 vs03b05.r1 Mus musculus cDNA, 5 end/clone = IMAGE-1137105/clone_end = 5/gb = AA669670/ | P | 571 | P | 53.1 | A | 161.5 | A |
| | | gi = 2690606/ug = Mm.24660/len = 238/STRA = for | | | | | | | |
| 20 | 95034_f_at | Cluster Incl AW212243 uo84d09 x1 Mus musculus cDNA, 3 end/clone = IMAGE-2649233/clone_end = 3/gb = AW212243/ | A | 253.5 | A | 237.5 | P | 64.9 | A |
| | | gi = 6518272/ug = Mm 27546/len = 250/STRA = for | | | | | | | |
| 20 | 95618_at | Clusler Incl AI843884 UI-M-AK1-aeu-e-10-0-UI s1 Mus musculus cDNA, 3 end/clone = UI-M-AK1-aeu-e-10-0-UI/ | P | 149.4 | P | 149.5 | A | 200.9 | P |
| | | clone_end = 3/gb = AI843884/gi = 5478097/ug = Mm.28032/len = 392/STRA = rev | | | | | | | |
| 20 | 103238_at | Cluster Incl M89797 Wingless-related MMTV integration site 4/cds = (45,1100)/gb = M89797/gi = 202401/ug = Mm 20355/ | P | 354.5 | P | 318.1 | A | 262.2 | A |
| | | len = 1101/STRA = for | | | | | | | |
| 20 | 103445_at | Cluster Incl M18401 Mus musculus putative homeobox 2.2 protein gene, partial cds/cds = (0,293)/gb = M18401/gi = 193938/ | P | 315.3 | P | 201.4 | A | 211.8 | P |
| | | ug = Mm.215/len = 294/STRA = for | | | | | | | |
| 20 | 104198_at | Cluster Incl AW211760 uo85c06 y1 Mus musculus cDNA, 5 end/clone = IMAGE-2649322/clone_end = 5/gb = AW211760/ | P | 406.5 | P | 184.5 | A | 1.6 | M |
| | | gi = 6517732/ug = Mm 27519/len = 462/STRA = for | | | | | | | |
| 20 | 104210_at | Cluster Incl AI839082 UI-M-AK0-adl-f-03-0-UI s1 Mus musculus cDNA, 3 end/clone = UI-M-AK0-adl-f-03-0-UI/clone_end = 3/ | P | 152.9 | P | 152.2 | P | 56.5 | M |
| | | gb = AI839082/gi = 5473295/ug = Mm.2775/len = 212/STRA = for | | | | | | | |
| 20 | 92276_at | Cluster Incl AB021861: Mus musculus mRNA for apoptosis signal-regulating kinase 2, complete cds/cds = (269,4138)/ | P | 251.5 | P | 322.7 | P | 238.1 | A |
| | | gb = AB021861/gi = 4996560/ug = Mm.36640/len = 4325/STRA = for | | | | | | | |
| 20 | 92280_at | Cluster Incl AA867778 vx16e04 r1 Mus musculus cDNA, 5 end/clone = IMAGE-1264638/clone_end = 5/gb = AA867778/ | P | 215.9 | P | 255.5 | P | 90.8 | A |
| | | gi = 2963223/ug = Mm 3678/len = 512/STRA = for | | | | | | | |
| 20 | 92759_at | Cluster Incl U43298 Laminin, beta 3/cds = (156,3662)/gb = U43298/gi = 1151214/ug = Mm 4732/len = 4049/STRA = for | P | 320.7 | P | 232.5 | A | 320.4 | P |
| 20 | 94791_s_at | Cluster Incl M90470 Leukocyte tyrosine kinase/cds = (137,2803)/gb = M90470/gi = 198885/ug = Mm.1740/len = 3077/ | P | 414.8 | P | 653.3 | P | 590.7 | P |
| | | STRA = for | | | | | | | |
| 20 | 96566_at | Cluster Incl L10385.Mus musculus transglutaminase E (TGASE3) mRNA, complete cds/cds = (54,2135)/gb = L10385/ | P | 284.2 | P | 164.7 | P | 190.4 | A |
| | | gi = 309520/ug = Mm 2961/len = 2297/STRA = for | | | | | | | |
| 20 | 98372_at | Cluster Incl AW050387 UI-M-BH1-ang-h-10-0-UI s1 Mus musculus cDNA, 3 end/clone = UI-M-BH1-ang-h-10-0-UI/ | P | 86.7 | P | 59.5 | P | 55.4 | P |
| | | clone_end = 3/gb = AW050387/gi = 5910916/ug = Mm.4458/len = 380/STRA = for | | | | | | | |
| 20 | 95793_at | Cluster Incl AJ005562 Small proline-rich protein 2D/cds = (43,300)/gb = AJ005562/gi = 3093362/ug = Mm 87820/len = 668/ | P | 1002.3 | P | 102.5 | A | 709.2 | P |
| | | STRA = for | | | | | | | |
| 20 | 92870_at | Cluster Incl AF063095.Mus musculus SEL1L (Sel1l) mRNA, complete cds/cds = (75,2297)/gb = AF063095/gi = 4159994/ | P | 207.3 | P | 131.2 | M | 122.5 | A |
| | | ug = Mm 14352/len = 3663/STRA = for | | | | | | | |
| 20 | 93105_s_at | Cluster Incl M26056 Mouse T-cell receptor germline beta-chain gene constant region (CT)/cds = (0,520)/gb = M26056/ | P | 266.4 | P | 254 | A | 247.5 | M |
| | | gi = 201239/ug = Mm 16654/len = 521/STRA = for | | | | | | | |
| 20 | 93795_at | Cluster Incl AW124626 UI-M-BH2 1-apw-b-07-0-UI.s1 Mus musculus cDNA, 3 end/clone = UI-M-BH2 1-apw-b-07-0-UI/ | P | 278.3 | P | 220.3 | M | 160.3 | A |
| | | clone_end = 3/gb = AW124626/gi = 6100156/ug = Mm 21399/len = 416/STRA = for | | | | | | | |
| 20 | 94260_at | Cluster Incl AI850352 UI-M-BG1-aig-a-08-0-UI s1 Mus musculus cDNA, 3 end/clone = UI-M-BG1-aig-a-08-0-UI/ | P | 540.9 | P | 506 | A | 455.7 | A |
| | | clone_end = 3/gb = AI850352/gi = 5494258/ug = Mm 22428/len = 420/STRA = for | | | | | | | |
| 20 | 94316_at | Cluster Incl AB012042 Mus musculus gene for keratin 6 beta/cds = (287,1975)/gb = AB012042/gi = 3845567/ug = Mm 22657/ | P | 1375.5 | A | 1182 | P | 183.5 | A |
| | | len = 2299/STRA = for | | | | | | | |
| 20 | 104601_at | Cluster Incl X14432 Thrombomodulin/cds = (158,1891)/gb = X14432/gi = 54781/ug = Mm 24096/len = 3636/STRA = for | P | 193.5 | P | 64.1 | A | 131.9 | P |
| 20 | 97420_at | Cluster Incl AW230891 uo69b04.y1 Mus musculus cDNA, 5 end/clone = IMAGE-2647759/clone_end = 5/gb = AW230891/ | P | 406.1 | P | 39.5 | A | −110.9 | A |
| | | gi = 6560187/ug = Mm 29986/len = 523/STRA = rev | | | | | | | |
| 20 | 100884_at | Cluster Incl U04204 Fibroblast growth factor regulated protein/cds = (67,1017)/gb = U04204/gi = 463376/ug = Mm 5378/ | P | 246.3 | P | 2.6 | A | 64.6 | A |
| | | len = 1304/STRA = for | | | | | | | |
| 20 | 101464_at | Cluster Incl V00755 Interferon beta, fibroblast/cds = (0,118)/gb = V00755/gi = 51554/ug = Mm 8245/len = 222/STRA = for | P | 1160.7 | P | 806.2 | P | 1239.4 | A |
| 20 | 102889_r_at | Cluster Incl X86569 LIM-domain containing, protein kinase/cds = (322,2265)/gb = X86569/gi = 1051159/ug = Mm.15409/ | P | 36.3 | P | 63.1 | A | 390.2 | P |
| | | len = 3439/STRA = for | | | | | | | |
| 20 | 93000_g_at | Cluster Incl D50416 Mouse mRNA for AREC3, complete cds/cds = (61,2388)/gb = D50416/gi = 1255625/ug = Mm.5085/ | P | 152.1 | P | 123.9 | A | 30.2 | P |
| | | len = 5566/STRA = for | | | | | | | |
| 20 | 93204_r_at | Cluster Incl AW107463 um17a12 x1 Mus musculus cDNA, 3 end/clone = IMAGE-2192542/clone_end = 3/gb = AW107463/ | P | 47.7 | P | 33.7 | M | 41.5 | A |
| | | gi = 6078263/ug = Mm.5697/len = 484/STRA = rev | | | | | | | |
| 20 | 97790_s_at | Cluster Incl X84014 Laminin, alpha 3/cds = (0,7710)/gb = X84014/gi = 1922890/ug = Mm.42012/len = 8077/STRA = for | P | 65.3 | P | 38.1 | A | −4.8 | A |
| 20 | 100333_at | Cluster Incl M13521 Murine serum amyloid A-1 (SAA-1) gene/cds = (0,368)/gb = M13521/gi = 200905/ug = Mm 87850/ | P | 533.2 | P | 311.4 | A | 542 | A |
| | | len = 369/STRA = for | | | | | | | |
| 20 | 92858_at | Cluster Incl AF002719 Mus musculus secretory leukoprotease inhibitor gene, complete cds/cds = (20,415)/gb = AF002719/ | P | 587.6 | P | 224.3 | A | −152.7 | A |
| | | gi = 4100898/ug = Mm 1395/len = 660/STRA = for | | | | | | | |
| 20 | 93603_at | Cluster Incl AF034092 Mus musculus nuclear localization signal containing protein (Nlvcf) mRNA, complete cds/ | P | 221.2 | P | 172 | A | 135.7 | A |
| | | cds = (114,734)/gb = AF034092/gi = 3834385/ug = Mm.20918/len = 874/STRA = for | | | | | | | |
| 20 | 96093_at | Cluster Incl AI842705 UI-M-AO1-aeo-d-09-0-UI s1 Mus musculus cDNA, 3 end/clone = UI-M-AO1-aeo-d-09-0-UI/ | A | 387.2 | P | 291.7 | P | 346 | A |
| | | clone_end = 3/gb = AI842705/gi = 5476918/ug = Mm.26740/len = 300/STRA = for | | | | | | | |
| 20 | 98892_at | Cluster Incl AI846934 UI-M-AK1-aey-e-06-0-UI s1 Mus musculus cDNA, 3 end/clone = UI-M-AK1-aey-e-06-0-UI/ | P | 100 | P | 113.7 | A | 46.9 | A |
| | | clone_end = 3/gb = AI846934/gi = 5490840/ug = Mm.28548/len = 407/STRA = for | | | | | | | |
| 20 | 104730_at | Cluster Incl AW046889: UI-M-BH1-akl-d-04-0-UI s1 Mus musculus cDNA, 3 end/clone = UI-M-BH1-akl-d-04-0-UI/ | P | 70.9 | P | 75.4 | A | −2.5 | A |
| | | clone_end = 3/gb = AW046889/gi = 5907418/ug = Mm 24627/len = 369/STRA = rev | | | | | | | |
| 20 | 94383_at | Cluster Incl D50586 Tissue factor pathway inhibitor 2/cds = (35,727)/gb = D50586/gi = 2467368/ug = Mm 25612/len = 1522/ | P | 82.7 | P | −22.3 | A | 6 | A |
| | | STRA = for | | | | | | | |
| 20 | 94924_at | Cluster Incl AW122679 UI-M-BH2 2-aol-h-11-0-UI s1 Mus musculus cDNA, 3 end/clone = UI-M-BH2.2-aol-h-11-0-UI/ | P | 667.8 | P | 408.8 | M | 338.6 | A |
| | | clone_end = 3/gb = AW122679/gi = 6098209/ug = Mm 2664/len = 291/STRA = for | | | | | | | |
| 20 | 96764_at | Cluster Incl AJ007971 Mus musculus mRNA for IIGP protein/cds = (104,1345)/gb = AJ007971/gi = 4158173/ug = Mm 29008/ | P | 681.1 | P | 48.4 | A | 12.6 | A |
| | | len = 2280/STRA = for | | | | | | | |
| 20 | 100976_at | Cluster Incl AF013490 Mus musculus protein-tyrosine phosphatase mRNA, complete cds/cds = (96,1877)/gb = AF013490/ | P | 460.6 | P | 357.1 | A | 255.4 | A |
| | | gi = 2331298/ug = Mm.6678/len = 3531/STRA = for | | | | | | | |
| 20 | 101384_at | Cluster Incl AI852933 UI-M-BH0-aiz-c-09-0-UI s1 Mus musculus cDNA, 3 end/clone = UI-M-BH0-aiz-c-09-0-UI/ | P | 331.9 | P | −130 | A | 178.8 | A |
| | | clone_end = 3/gb = AI852933/gi = 5496839/ug = Mm.7114/len = 395/STRA = for | | | | | | | |
| 20 | 102689_at | Cluster Incl AF110520 Mus musculus major histocompatibility complex region NG27, NG28, RPS28, NADH | P | 334.6 | P | 293.6 | A | 262.7 | A |
| | | oxidoreductase, NG29, KIFC1, Fas-binding protein, BING1, tapasin, RalGDS-like, KE2, BING4, beta 1,3-galactosyl | | | | | | | |
| | | transferase, and RPS18 genes, complete cds, S/STRA = for | | | | | | | |
| 20 | 102712_at | Cluster Incl X03505 Serum amyloid A 3/cds = (0,368)/gb = X03505/gi = 54028/ug = Mm 14277/len = 369/STRA = for | P | 540.8 | P | 5.1 | A | 1.8 | A |
| 20 | 104035_at | Cluster Incl AW123795 UI-M-BH2.1-apm-e-02-0-UI s1 Mus musculus cDNA, 3 end/clone = UI-M-BH2 1-apm-e-02-0-UI/ | P | 57.2 | M | −20.9 | A | −61.6 | P |
| | | clone_end = 3/gb = AW123795/gi = 6099325/ug = Mm 27057/len = 404/STRA = rev | | | | | | | |
| 20 | 93145_at | Cluster Incl AI840824 UI-M-AH0-adb-d-03-0-UI s1 Mus musculus cDNA, 3 end/clone = UI-M-AH0-adb-d-03-0-UI/ | P | 90.4 | P | 102 | A | 67.6 | M |
| | | clone_end = 3/gb = AI840824/gi = 5475037/ug = Mm 5209/len = 447/STRA = for | | | | | | | |
| 20 | 93878_at | Cluster Incl AF010135 Mus musculus maf10 (mAF10) mRNA, complete cds/cds = (178,3384)/gb = AF010135/gi = 2809380/ | P | 194.6 | P | 243.5 | P | 154.7 | P |
| | | ug = Mm 8866/len = 3543/STRA = for | | | | | | | |
| 20 | 100308_at | Cluster Incl X66976.M. musculus col8a1 gene, exon 1 (and joined CDS)/cds = (0,2231)/gb = X66976/gi = 50493/ | P | 88.2 | A | 160 | P | 117.2 | A |
| | | ug = Mm 86813/len = 2232/STRA = for | | | | | | | |
| 20 | 97598_at | Cluster Incl AW209139: uo02d07.x1 Mus musculus cDNA, 3 end/clone = IMAGE-2582221/clone_end = 3/gb = AW209139/ | P | 9.4 | P | 17.8 | A | 53.4 | A |
| | | gi = 6515079/ug = Mm.89650/len = 425/STRA = rev | | | | | | | |
| 20 | 102423_at | Cluster Incl AI649125 uk31e12 x1 Mus musculus cDNA, 3 end/clone = IMAGE-1970638/clone_end = 3/gb = AI649125/ | P | 45.7 | P | 45.1 | A | 35.2 | A |
| | | gi = 4729959/ug = Mm.12790/len = 593/STRA = for | | | | | | | |
| 20 | 102763_at | Cluster Incl AF064748 Mus musculus S3-12 mRNA, complete cds/cds = (138,4349)/gb = AF064748/gi = 3236367/ | P | 59.1 | P | 71.4 | A | −105.4 | A |
| | | ug = Mm 12966/len = 5774/STRA = for | | | | | | | |
| 20 | 103080_at | Cluster Incl U15635 Mus musculus IFN-gamma induced (Mg11) mRNA, complete cds/cds = (57,1841)/gb = U15635/ | P | 345.7 | P | 79.1 | A | 49.8 | A |
| | | gi = 558902/ug = Mm.17167/len = 1942/STRA = for | | | | | | | |
| 20 | 103634_at | Cluster Incl U51992.lnterferon dependent positive acting transcription factor 3 gamma/cds = (64,1263)/gb = U51992/ | P | 408 | P | 3 | A | 131.5 | A |
| | | gi = 1263309/ug = Mm 2032/len = 1959/STRA = for | | | | | | | |
| 20 | 104293_at | Cluster Incl AI882440 uc01f05 r1 Mus musculus cDNA, 5 end/clone = IMAGE-1396737/clone_end = 5/gb = AI882440/ | P | 92.4 | P | 61.8 | A | −165.7 | A |
| | | gi = 5567529/ug = Mm 22306/len = 271/STRA = for | | | | | | | |
| 20 | 96123_at | Cluster Incl X99347 M musculus mRNA for LPS-binding protein/cds = (127,1572)/gb = X99347/gi = 1430866/ug = Mm 28110/ | P | 106.1 | M | 96.1 | A | 109.6 | A |
| | | len = 1925/STRA = for | | | | | | | |
| 20 | 96766_s_at | Cluster Incl AB000828 Mouse mRNA for receptor tyrosine kinase, complete cds/cds = (13,2655)/gb = AB000828/ | P | 251.5 | P | 111.3 | A | 154.2 | A |
| | | gi = 1871412/ug = Mm 2901/len = 2845/STRA = for | | | | | | | |
| 20 | 97971_at | Cluster Incl AI852148 UI-M-BH0-ajb-d-12-0-UI s1 Mus musculus cDNA, 3 end/clone = UI-M-BH0-ajb-d-12-0-UI/ | P | 199.8 | A | 235.6 | A | 91.2 | A |
| | | clone_end = 3/gb = AI852148/gi = 5496049/ug = Mm 30991/len = 375/STRA = for | | | | | | | |
| 20 | 98501_at | Cluster Incl Y07519 Lymphocyte antigen 84/cds = (194,1207)/gb = Y07519/gi = 55517/ug = Mm 35692/len = 2646/STRA = for | P | 36.5 | P | −28.4 | A | −16.4 | A |
| 20 | 100484_at | Cluster Incl X66473 Matrix metalloproteinase 1/cds = (10,1428)/gb = X66473/gi = 53603/ug = Mm 5022/len = 2660/STRA = for | P | 115.2 | P | 42.2 | A | 37.7 | A |
| 20 | 100983_at | Cluster Incl AB022053 Mus musculus gene for prolyl oligopeptidase/cds = (377,2509)/gb = AB022053/gi = 5689034/ | P | 250.6 | P | 209.6 | M | 289 | M |
| | | ug = Mm 6735/len = 2981/STRA = for | | | | | | | |
| 20 | 102637_at | Cluster Incl AF039601 Mus musculus betaglycan mRNA, complete cds/cds = (231,2783)/gb = AF039601/gi = 3387819/ | P | 92.7 | A | 25.8 | A | 64.1 | M |
| | | ug = Mm 12960/len = 3977/STRA = rev | | | | | | | |
| 20 | 103446_at | Cluster Incl AA959954 vw53a09 s1 Mus musculus cDNA, 3 end/clone = IMAGE-1247512/clone_end = 3/gb = AA959954/ | P | 239.1 | P | −41 | A | −124 | A |
| | | gi = 3125854/ug = Mm 21551/len = 524/STRA = for | | | | | | | |
| 20 | 92234_at | Cluster Incl X66223 M musculus mRNA for retinoid X receptor-alpha (mRXR-alpha)/cds = (177,1580)/gb = X66223/ | P | 294.1 | P | 334.4 | A | 177.4 | A |
| | | gi = 54021/ug = Mm 3470/len = 2088/STRA = for | | | | | | | |
| 20 | 92353_at | Cluster Incl Y18723 Mus musculus mRNA for secreted senne protease (BSSP)/cds = (237,977)/gb = Y18723/gi = 4220545/ | P | 907.5 | P | 255.4 | A | −166.7 | A |
| | | ug = Mm 3944/len = 1122/STRA = rev | | | | | | | |
| 20 | 93866_s_at | Cluster Incl D00613 Matrix gamma-carboxyglutamate (gia) protein/cds = (18,332)/gb = D00613/gi = 220478/ug = Mm 87793/ | P | 197 | A | 126.5 | M | 178.5 | A |
| | | len = 540/STRA = for | | | | | | | |
| 20 | 94120_s_at | Cluster Incl AJ005564 Small proline-nch protein 2F/cds = (23,253)/gb = AJ005564/gi = 3093366/ug = Mm 10692/len = 570/ | P | 279.1 | P | 133.2 | M | 124.9 | A |
| | | STRA = for | | | | | | | |
| 20 | 98772_at | Cluster Incl U27267.Small inducible cytokine B subfamily, member 5/cds = (65,463)/gb = U27267/gi = 950158/ug = Mm 4660/ | P | 125.8 | P | 103.3 | A | 34.3 | A |
| | | len = 1519/STRA = for | | | | | | | |
| 20 | 98833_at | Cluster Incl X66402 Matrix metalloproteinase 3/cds = (24,1457)/gb = X66402/gi = 296167/ug = Mm.4993/len = 1740/ | P | 158.4 | P | 68.9 | A | 64.6 | A |
| | | STRA = for | | | | | | | |

| | Affymetrix | | | | | | | |
|---|---|---|---|---|---|---|---|---|
| | accession | | PU.1 | | PU.1 KO | | PU.1 KO | |
| Cluster | number | Description | KO 3 hours | Call | 12 hours | Call | 24 hours | Call |
| 1 | 92769_at | Cluster Incl M25149: Tissue specific transplantation antigen P91A/cds = (0,1589)/gb = M25149/gi = 191974/ug = Mm 12194/ | 970.2 | P | 1004.4 | P | 843.8 | P |
| | | len = 1590/STRA = for | | | | | | |
| 1 | 93565_at | Cluster Incl AF055665: Kinesin light chain 1/cds = (94,1719)/gb = AF055665/gi = 3347845/ug = Mm 20354/len = 2289/ | 247.2 | P | 316.2 | P | 159.5 | P |
| | | STRA = for | | | | | | |
| 1 | 93589_at | Cluster Incl AI851172: UI-M-BH0-akg-g-03-0-UI s1 Mus musculus cDNA, 3 end/clone = UI-M-BH0-akg-g-03-0-UI/ | 984.3 | P | 987.8 | P | 924.3 | P |
| | | clone_end = 3/gb = AIB51172/gi = 5495043/ug = Mm 20806/len = 363/STRA = for | | | | | | |
| 1 | 93749_at | Cluster Incl AI848045: UI-M-AH1-agp-g-05-0-UI s1 Mus musculus cDNA, 3 end/clone = UI-M-AH1-agp-g-05-0-UI/ | 272.4 | P | 251.5 | P | 218.8 | P |
| | | clone_end = 3/gb = AI848045/gi = 5491951/ug = Mm 21108/len = 224/STRA = for | | | | | | |
| 1 | 93757_at | Cluster Incl AI843574 UI-M-AO1-aen-d-12-0-UI s1 Mus musculus cDNA, 3 end/clone = UI-M-AO1-aen-d-12-0-UI/ | 1427.8 | P | 1536 | P | 1091.1 | P |
| | | clone_end = 3/gb = AI843574/gi = 5477827/ug = Mm 21132/len = 352/STRA = for | | | | | | |
| 1 | 94024_at | Cluster Incl AA671429 vn81g12 r1 Mus musculus cDNA, 5 end/clone = IMAGE-1038406/clone_end = 5/gb = AA671429/ | 1366.4 | P | 1202.1 | P | 1108.4 | P |
| | | gi = 2643508/ug = Mm 21873/len = 464/STRA = for | | | | | | |
| 1 | 94254_at | Cluster Incl AI845237 UI-M-BG0-aht-g-09-0-UI s1 Mus musculus cDNA, 3 end/clone = UI-M-BG0-aht-g-09-0-UI/ | 1416.4 | P | 1468 | P | 1247.2 | P |
| | | clone_end = 3/gb = AI845237/gi = 5489143/ug = Mm 22409/len = 255/STRA = for | | | | | | |
| 1 | 94898_at | Cluster Incl AI848555 UI-M-AP1-agf-h-08-0-UI.s2 Mus musculus cDNA, 3 end/clone = UI-M-AP1-agf-h-08-0-UI/ | 383.9 | P | 364 | P | 279.6 | P |
| | | clone_end = 3/gb = AI848555/gi = 5492461/ug = Mm 24015/len = 426/STRA = for | | | | | | |
| 1 | 96001_at | Cluster Incl AW122293 UI-M-BH2 2-aos-c-01-0-UI s1 Mus musculus cDNA, 3 end/clone = UI-M-BH2 2-aos-c-01-0-UI/ | 1285.2 | P | 1405.7 | P | 998.5 | P |
| | | clone_end = 3/gb = AW122293/gi = 6097778/ug = Mm 25321/len = 363/STRA = for | | | | | | |
| 1 | 96045_at | Cluster Incl AW050142 UI-M-BH1-ano-e-11-0-UI.s1 Mus musculus cDNA, 3 end/clone = UI-M-BH1-ano-e-11-0-UI/ | 250.3 | P | 336.5 | P | 233.2 | P |
| | | clone_end = 3/gb = AW050142/gi = 5910671/ug = Mm 25994/len = 458/STRA = for | | | | | | |
| 1 | 96672_at | Cluster Incl AW123564 UI-M-BH2.1-aqb-h-01-0-UI.s1 Mus musculus cDNA, 3 end/clone = UI-M-BH2.1-aqb-h-01-0-UI/ | 2319.4 | P | 2845.1 | P | 2319.6 | P |
| | | clone_end = 3/gb = AW123564/gi = 6099094/ug = Mm 27401/len = 365/STRA = for | | | | | | |
| 1 | 96716_at | Cluster Incl AW121102: UI-M-BH2 3-aoh-b-01-0-UI s1 Mus musculus cDNA, 3 end/clone = UI-M-BH2 3-aoh-b-01-0-UI/ | 741.8 | P | 689.3 | P | 569.6 | P |
| | | clone_end = 3/gb = AW121102/gi = 6096435/ug = Mm 27647/len = 353/STRA = rev | | | | | | |
| 1 | 97226_at | Cluster Incl AW125649: UI-M-BH2.2-aqi-b-03-0-UI s1 Mus musculus cDNA, 3 end/clone = UI-M-BH2 2-aqi-b-03-0-UI/ | 309.1 | P | 402 | P | 369.7 | P |
| | | clone_end = 3/gb = AW125649/gi = 6101179/ug = Mm 27773/len = 435/STRA = for | | | | | | |
| 1 | 97263_s_at | Clusler Incl AI846289 UI-M-AK1-aez-f-02-0-UI s1 Mus musculus cDNA, 3 end/clone = UI-M-AK1-aez-f-02-0-UI/ | 1298 | P | 1498.6 | P | 1162.3 | P |
| | | clone_end = 3/gb = AI846289/gi = 5490195/ug = Mm 27899/len = 375/STRA = for | | | | | | |
| 1 | 97284_at | Clusler Incl AI853789 UI-M-BH0-aig-f-10-0-UI s1 Mus musculus cDNA, 3 end/clone = UI-M-BH0-aig-f-10-0-UI/ | 746.2 | P | 747.7 | P | 621.2 | P |
| | | clone_end = 3/gb = AI853789/gi = 5497695/ug = Mm 27990/len = 458/STRA = for | | | | | | |
| 1 | 97817_at | Cluster Incl AW121136 UI-M-BH2 3-aoh-e-05-0-UI s1 Mus musculus cDNA, 3 end/clone = UI-M-BH2 3-aoh-e-05-0-UI/ | 1035.2 | P | 1157.6 | P | 846.4 | P |
| | | clone_end = 3/gb = AW121136/gi = 6096469/ug = Mm 28189/len = 391/STRA = for | | | | | | |
| 1 | 97823_g_at | Cluster Incl AW122689 UI-M-BH2 2-aot-a-10-0-UI s1 Mus musculus cDNA, 3 end/clone = UI-M-BH2 2-aot-a-10-0-UI/ | 735.3 | P | 808.1 | P | 729.4 | P |
| | | clone_end = 3/gb = AW122689/gi = 6098219/ug = Mm 28202/len = 467/STRA = rev | | | | | | |
| 1 | 97871_at | Cluster Incl AW120614 UI-M-BH2 3-anx-g-02-0-UI s1 Mus musculus cDNA, 3 end/clone = UI-M-BH2 3-anx-g-02-0-UI/ | 496.6 | P | 652.6 | P | 327 | P |
| | | clone_end = 3/gb = AW120614/gi = 6095947/ug = Mm.28339/len = 294/STRA = rev | | | | | | |
| 1 | 98882_s_at | Cluster Incl AW047320 UI-M-BH1-amh-g-06-0-UI s1 Mus musculus cDNA, 3 end/clone = UI-M-BH1-amh-g-06-0-UI/ | 1569.7 | P | 1708.9 | P | 1882.1 | P |
| | | clone_end = 3/gb = AW047320/gi = 5907849/ug = Mm.28527/len = 616/STRA = for | | | | | | |
| 1 | 94451_at | Cluster Incl AI787627 UI20f06 y1 Mus musculus cDNA, 5 end/clone = IMAGE-2088131/clone_end = 5/gb = AI787627/ | 636.7 | P | 715.7 | P | 681.7 | P |
| | | gi = 5335343/ug = Mm 28786/len = 450/STRA = for | | | | | | |
| 1 | 95666_at | Cluster Incl AW123404 UI-M-BH2 1-apj-a-09-0-UI s1 Mus musculus cDNA, 3 end/clone = UI-M-BH2 1-apj-a-09-0-UI/ | 687.4 | P | 614.4 | P | 316 | P |
| | | clone_end = 3/gb = AW123404/gi = 6098934/ug = Mm 29572/len = 354/STRA = for | | | | | | |
| 1 | 95756_at | Clusler Incl AI839681: UI-M-AN0-acp-d-02-0-UI s1 Mus musculus cDNA, 3 end/clone = UI-M-AN0-acp-d-02-0-UI/ | 441.3 | P | 559.1 | P | 498.9 | P |
| | | clone_end = 3/gb = AI839681/gi = 5473894/ug = Mm 29795/len = 348/STRA = for | | | | | | |
| 1 | 96301_at | Clusler Incl AI851912 UI-M-BH0-aix-e-03-0-UI s1 Mus musculus cDNA, 3 end/clone = UI-M-BH0-aix-e-03-0-UI/ | 698.7 | P | 1285.3 | P | 654.9 | P |
| | | clone_end = 3/gb = AI851912/gi = 5495818/ug = Mm 29911/len = 392/STRA = for | | | | | | |
| 1 | 96357_at | Cluster Incl AW212775 uo66e09.x1 Mus musculus cDNA, 3 end/clone = IMAGE-2647528/clone_end = 3/gb = AW212775/ | 750.4 | P | 852.4 | P | 510.7 | P |
| | | gi = 6518896/ug = Mm 30010/len = 165 STRA = for | | | | | | |
| 1 | 97550_at | Cluster Incl AW047228 UI-M-BH1-amb-f-04-0-UI.s1 Mus musculus cDNA, 3 end/clone = UI-M-BH1-amb-f-04-0-UI/ | 820 | P | 858.7 | P | 679.4 | P |
| | | clone_end = 3/gb = AW047228/gi = 5907757/ug = Mm.33797/len = 314/STRA = for | | | | | | |
| 1 | 99559_at | Cluster Incl U14390 Aldehyde dehydrogenase 4, liver microsomal (class 3)/cds = (113,1587)/gb = U14390/gi = 687253/ | 1070.6 | P | 1195.5 | P | 860.9 | P |
| | | ug = Mm 4210/len = 2997/STRA = for | | | | | | |
| 1 | 100566_at | Cluster Incl L12447 Insulin-like growth factor binding protein 5/cds = (387,1202)/gb = L12447/gi = 425405/ug = Mm.578/ | 575.9 | P | 1207.4 | P | 826.6 | P |
| | | len = 5561/STRA = for | | | | | | |
| 1 | 101490_at | Cluster Incl AW122692 UI-M-BH2 2-aot-b-01-0-UI s1 Mus musculus cDNA, 3 end/clone = UI-M-BH2 2-aol-b-01-0-UI/ | 518.2 | P | 579.3 | P | 382.1 | P |
| | | clone_end = 3/gb = AW122692/gi = 6098222/ug = Mm 7884/len = 288/STRA = for | | | | | | |
| 1 | 101520_at | Cluster Incl AF032967 Mus musculus strain C3H/HeN mRNA preferentially expressed In LPS-normoresponsive | 377.1 | P | 358.4 | P | 315.8 | P |
| | | macrophages/cds = UNKNOWN/gb = AF032967/gi = 4103990/ug = Mm.8540/len = 1766/STRA = for | | | | | | |
| 1 | 101579_at | Clusler Incl X78304: M. musculus (SRP9) signal recognition particle subunit mRNA, 689bp/cds = (93,353)/gb = X78304/ | 604.1 | P | 601.2 | P | 419.7 | P |
| | | gi = 872130/ug = Mm 89927/len = 689/STRA = for | | | | | | |
| 1 | 102324_at | Cluster Incl AF036875 Mus musculus multiple endocrine neoplasia type 1 candidate prolein number 18 mRNA, complete | 259.2 | P | 337.7 | P | 176.6 | P |
| | | cds/cds = (19,753)/gb = AF036875/gi = 3136115/ug = Mm 10721/len = 841/STRA = for | | | | | | |
| 1 | 102768_l_at | Cluster Incl AB016248 Mus musculus mRNA for sterol-C5-desaturase, complete cds/cds = (170,1069)/gb = AB016248/ | 104.6 | P | 147.1 | P | 95.9 | P |
| | | gi = 3721883/ug = Mm.13081/len = 1721/STRA = for | | | | | | |
| 1 | 102993_at | Cluster Incl M85153: Glycoprotein galactosyltransferase alpha 1, 3/cds = (444,1559)/gb = M85153/gi = 193563/ug = Mm.1548/ | 754.8 | P | 903.5 | P | 770.3 | P |
| | | len = 3450/STRA = for | | | | | | |
| 1 | 103005_s_at | Cluster Incl X66084 CD44 antigen/cds = (9,1976)/gb = X66084/gi = 53679/ug = Mm 15643/len = 1997/STRA = for | 409.9 | P | 442.7 | H | 238 | P |
| 1 | 103052_r_at | Cluster Incl X76653 Apolipoprotein regulatory protein 1/cds = (35,1279)/gb = X76653/gi = 482927/ug = Mm.16519/len = 2495/ | 166.2 | P | 189.9 | P | 177 | P |
| | | STRA = for | | | | | | |
| 1 | 103345_at | Cluster Incl AW046708: UI-M-BH1-alt-d-05-0-Ul.s1 Mus musculus cDNA, 3 end/clone = UI-M-BH1-alt-d-05-0-UI/ | 574.1 | P | 866.4 | P | 577.7 | P |
| | | clone_end = 3/gb = AW046708/gi = 5907277/ug = Mm. 18249/len = 412/STRA = for | | | | | | |
| 1 | 103669_at | Cluster Incl AA793857: vr40b03.s1 Mus musculus cDNA, 5 end/clone = IMAGE-1123085/clone_end = 5/gb = AA793857/ | 426.6 | P | 588.1 | P | 431.4 | P |
| | | gi = 2856812/ug = Mm.2077/len = 261/STRA = for | | | | | | |
| 1 | 103814_at | Cluster Incl AF093871.Mus musculus peroxisomal biogenesis factor (Pex11b) mRNA, complete cds/cds = (3,782)/ | 568.9 | P | 562.8 | P | 493.5 | P |
| | | gb = AF093671/gi = 3907595/ug = Mm 20901/len = 1109/STRA = for | | | | | | |
| 1 | 103832_at | Cluster Incl AF097181 Mus musculus tuftelin-interacting protein 10 mRNA, complete cds/cds = (96,1676)/gb = AF097181/ | 400.3 | P | 405.1 | P | 364.1 | P |
| | | gi = 3851163/ug = Mm.20933/len = 2424/STRA = for | | | | | | |
| 1 | 104069_at | Cluster Incl AI852737 UI-M-BH0-aji-h-05-0-UI.s1 Mus musculus cDNA, 3 end/clone = UI-M-BH0-aji-h-05-0-UI/clone_end = 3/ | 1084.2 | P | 1349.9 | P | 1048.5 | P |
| | | gb = AI852737/gi = 5496643/ug = Mm 21619/len = 503/STRA = rev | | | | | | |
| 1 | 104091_at | Cluster Incl AW122563 UI-M-BH2 2-aox-e-09-0-UI s1 Mus musculus cDNA, 3 end/clone = UI-M-BH2 2-aox-e-09-0-UI/ | 957.4 | P | 883.3 | P | 805 | P |
| | | clone_end = 3/gb = AW122563/gi = 6098087/ug = Mm 21814/len = 274/STRA = for | | | | | | |
| 1 | 104128_at | Cluster Incl AW048075 UI-M-BH1-alq-a-05-0-UI s1 Mus musculus cDNA, 3 end/clone = UI-M-BH1-alq-a-05-0-UI/ | 1149.5 | P | 1204.2 | P | 1212.5 | P |
| | | clone_end = 3/gb = AW048075/gi = 5908604/ug = Mm 22040/len = 340/STRA = for | | | | | | |
| 1 | 104574_at | Cluster Incl AW210607.um62e04.x1 Mus musculus cDNA, 3 end/clone = IMAGE-2285982/clone_end = 3/gb = AW210607/ | 612 | P | 711 | P | 623 | P |
| | | gi = 6516547/ug = Mm 23927/len = 418/STRA = rev | | | | | | |
| 1 | 94973_at | Cluster Incl AA840126 ud01d09 r1 Mus musculus cDNA, 5 end/clone = lMAGE-1433873/clone_end = 5/gb = AA840126/ | 273.8 | P | 217.9 | P | 205.6 | P |
| | | gi = 2915835/ug = Mm 27232/len = 471/STRA = for | | | | | | |
| 1 | 95010_at | Cluster Incl U21050 Tnf receptor-associated factor 3/cds = (180,1883)/gb = U21050/gi = 719292/ug = Mm.27431/len = 2359/ | 275.4 | P | 295 | P | 261.4 | P |
| | | STRA = for | | | | | | |
| 1 | 95563_at | Cluster Incl AJ130977 Mus musculus mRNA for Ariadne protein, partial/cds = (0,1394)/gb = AJ130977/gi = 3925718/ | 93.7 | P | 214.1 | P | 167.2 | P |
| | | ug = Mm 27783/len = 1629/STRA = for | | | | | | |
| 1 | 96183_at | Cluster Incl AW122985: UI-M-BH2 1-apb-b-02-0-UI s1 Mus musculus cDNA, 3 end/clone = UI-M-BH2 1-apb-b-02-0-UI/ | 249.3 | P | 269.1 | P | 211.5 | P |
| | | clone_end = 3/gb = AW122985/gi = 6098515/ug = Mm 28459/len = 427/STRA = for | | | | | | |
| 1 | 96219_at | Cluster Incl AI850357: UI-M-BG1-aig-b-01-0-UI s1 Mus musculus cDNA. 3 end/clone = UI-M-BG1-aig-b-01-0-UI/ | 828.5 | P | 1030.4 | P | 953.9 | P |
| | | clone_end = 3/gb = AIB50357/gi = 5494263/ug = Mm 28702/len = 456/STRA = for | | | | | | |
| 1 | 96725_at | Cluster Incl AI838398. UI-M-AJ0-aav-h-02-0-UI s2 Mus musculus cDNA, 3 end/clone = UI-M-AJ0-aav-h-02-0-UI/ | 556.9 | P | 653 | P | 516.4 | P |
| | | clone_end = 3/gb = AI838398/gi = 5472611/ug = Mm 28833/len = 260/STRA = for | | | | | | |
| 1 | 96749_f_at | Cluster Incl AA619554 vo88e08 r1 Mus musculus cDNA, 5 end/clone = IMAGE-1066214/clone_end = 5/gb = AA619554/ | 38 | P | 60 | P | 38.7 | P |
| | | gi = 2523430/ug = Mm 28958/len = 468/STRA = for | | | | | | |
| 1 | 96832_at | Cluster Incl AA606878 vm87a04 r1 Mus musculus cDNA, 5 end/clone = IMAGE-1005198/clone_end = 5/gb = AA606878/ | 905.4 | P | 1027.9 | P | 913.4 | P |
| | | gi = 2455771/ug = Mm 29419/len = 223/STRA = for | | | | | | |
| 1 | 97392_at | Cluster Incl AW050086 UI-M-BH1-amm-g-09-0-UI s1 Mus musculus cDNA, 3 end/clone = UI-M-BH1-amm-g-09-0-UI/ | 577.1 | P | 540.5 | P | 485.4 | P |
| | | clone_end = 3/gb = AW050086/gi = 5910615/ug = Mm 29809/len = 442/STRA = for | | | | | | |
| 1 | 97395_at | Cluster Incl AW122465 UI-M-BH2 2-aou-c-10-0-UI s1 Mus musculus cDNA, 3 end/clone = UI-M-BH2 2-aou-c-10-0-UI/ | 1276.7 | P | 1181.7 | P | 1143.5 | P |
| | | clone_end = 3/gb = AW122465/gi = 6097985/ug = Mm 29835/len = 221/STRA = for | | | | | | |
| 1 | 97917_at | Cluster Incl Y13778 Mus musculus GCN5L1 gene, exon 1 and joined CDS/cds = (70,447)/gb = Y13778/gi = 2769584/ | 1510 | P | 1436.3 | P | 1388.1 | P |
| | | ug = Mm 30118/len = 448/STRA = for | | | | | | |
| 1 | 97957_at | Cluster Incl AF072759 Mus musculus fatty acid transport protein 4 mRNA, partial cds/cds = (0,1520)/gb = AF072769/ | 525.1 | P | 732 | P | 541.2 | P |
| | | gi = 3335568/ug = Mm.30487/len = 1521/STRA = for | | | | | | |
| 1 | 98439_at | Cluster Incl AW049358 UI-M-BH1-ane-a-03-0-UI s1 Mus musculus cDNA, 3 end/clone = UI-M-BH1-ane-a-03-0-UI/ | 421.8 | P | 489.2 | P | 404.6 | P |
| | | clone_end = 3/gb = AW049356/gi = 5909885/ug = Mm 34490/len = 437/STRA = for | | | | | | |
| 1 | 99033_at | Cluster Incl AI837130 UI-M-AK0-adc-g-01-0-UI.s1 Mus musculus cDNA, 3 end/clone = UI-M-AK0-adc-g-01-0-UI/ | 1302 | P | 1273.3 | P | 1005.8 | P |
| | | clone_end = 3/gb = AI837130/gi = 5471343/ug = Mm.39038/len = 406/STRA = for | | | | | | |
| 1 | 99068_at | Cluster Incl X80169 M musculus mRNA for 200 kD protein/cds = (252,6086)/gb = X80169/gi = 562764/ug = Mm 3989/ | 332.4 | P | 514.4 | P | 409.3 | P |
| | | len = 6331/STRA = for | | | | | | |
| 1 | 99445_at | Cluster Incl AW047012 UI-M-BH1-alp-g-12-0-UI s1 Mus musculus cDNA, 3 end/clone = UI-M-BH1-alp-g-12-0-UI/ | 426.2 | P | 473.4 | P | 318 | P |
| | | clone_end = 3/gb = AW047012/gi = 5907541/ug = Mm 4042/len = 373/STRA = for | | | | | | |
| 1 | 99492_at | Cluster Incl AI851990. UI-M-BH0-aiq-e-01-0-UI.s1 Mus musculus cDNA, 3 end/clone = UI-M-BH0-aiq-e-01-0-UI/ | 557.6 | P | 468 | P | 444.3 | P |
| | | clone_end = 3/gb = AI851990/gi = 5495896/ug = Mm.41550/len = 404/STRA = for | | | | | | |
| 1 | 100010_at | Cluster Incl U36340 Mus musculus CACCC-box binding protein BKLF mRNA, complete cds/cds = (44,1078)/gb = U36340/ | 601.8 | P | 499.6 | P | 283.6 | P |
| | | gi = 1244514/ug = Mm 4549/len = 1200/STRA = for | | | | | | |
| 1 | 101837_g_at | Cluster Incl D45859 Protein phosphatase 1B, magnesium dependent, beta isoform/cds = (0,461)/gb = D45859/gi = 961469/ | 354.2 | P | 317.8 | P | 238.5 | P |
| | | ug = Mm 849/len = 546/STRA = for | | | | | | |
| 1 | 101858_at | Cluster Incl AF015770 Mus musculus radical Innge (radical-fringe) mRNA, complete cds/cds = (0,998)/gb = AF015770/ | 649.3 | P | 710.2 | P | 689.8 | P |
| | | gi = 2454573/ug = Mm 871/len = 999/STRA = for | | | | | | |
| 1 | 101933_at | Cluster Incl AF035646 Mus musculus small GTPase Rab10 (Rab10) mRNA, complete cds/cds = (241,843)/gb = AF035646/ | 538.3 | P | 718.1 | P | 475 | P |
| | | gi = 3406427/ug = Mm 9455/len = 897/STRA = for | | | | | | |
| 1 | 101938_at | Cluster Incl X75959 M. musculus mRNA for polyA binding protein (testis-enriched isoform)/cds = (163,2049)/gb = X75959/ | 348.4 | P | 437.9 | P | 342.7 | P |
| | | gi = 473091/ug = Mm 9493/len = 2135/STRA = for | | | | | | |
| 1 | 102306_at | Cluster Incl AF060178 Mus musculus heparan sulfate 2-sulfotransferase (Hs2st) mRNA, complete cds/cds = (37,1107)/ | 237.5 | P | 271.8 | P | 255.4 | P |
| | | gb = AF060178/gi = 3228535/ug = Mm 12863/len = 2057/STRA = for | | | | | | |
| 1 | 102960_at | Cluster Incl X96618 M. musculus mRNA for novel stromal cell protein/cds = (96,761)/gb = X96618/gi = 1370221/ | 348.4 | P | 341.3 | P | 278.5 | P |
| | | ug = Mm.17958/len = 1031/STRA = for | | | | | | |
| 1 | 103219_at | Cluster Incl AI327518: mb43a09 y1 Mus musculus cDNA, 5 end/clone = IMAGE-332152/clone_end = 5/gb = AI327518/ | 740.7 | P | 835.9 | P | 735.5 | P |
| | | gi = 4061947/ug = Mm 19941/len = 423/STRA = for | | | | | | |
| 1 | 103439_at | Cluster Incl AW045417: UI-M-BH1-akr-a-01-0-UI.s1 Mus musculus cDNA, 3 end/clone = UI-M-BH1-akr-a-01-0-UI/ | 735.3 | P | 1170 | P | 870.6 | P |
| | | clone_end = 3/gb = AW045417/gi = 5905946/ug = Mm 214/len = 336/STRA = rev | | | | | | |
| 1 | 103451_at | Cluster Incl AI835159: UI-M-AQ0-aaf-e-10-0-UI S1 Mus musculus cDNA, 3 end/clone = UI-M-AQ0-aaf-e-10-0-UI/ | 343 | P | 414.7 | P | 303.1 | P |
| | | clone_end = 3/gb = AI835159/gi = 5469372/ug = Mm 21613/len = 139/STRA = for | | | | | | |
| 1 | 103751_at | Cluster Incl AA833096 uc95g05.r1 Mus musculus cDNA, 5 end/clone = IMAGE-1433432/clone_end = 5/gb = AA833096/ | 1287.8 | P | 1591 | P | 1192.7 | P |
| | | gi = 2906824/ug = Mm 24508/len = 289/STRA = for | | | | | | |
| 1 | 103790_at | Cluster Incl AI846632: UI-M-AQ1-aef-h-03-0-UI.s1 Mus musculus cDNA, 3 end/clone = UI-M-AQ1-aef-h-03-0-UI/ | 1164.4 | P | 1131.2 | P | 1065.3 | P |
| | | clone_end = 3/gb = AI846632/gi = 5490538/ug = Mm.24950/len = 322/STRA = for | | | | | | |
| 1 | 103971_at | Cluster Incl X84896: M. musculus mRNA for ATP receptor/cds = (164,1363)/gb = X84896/gi = 1166454/ug = Mm.25722/ | 505.9 | P | 732.6 | P | 387.9 | P |
| | | len = 1760/STRA = for | | | | | | |
| 1 | 104467_at | Cluster Incl AA763004: vw60b02 r1 Mus musculus cDNA, 5 end/clone = IMAGE-1248171/clone_end = 5/gb = AA763004/ | 779.1 | P | 722.7 | P | 727.8 | P |
| | | gl = 2812751/ug = Mm 2975/len = 545/STRA = for | | | | | | |
| 1 | 104492_at | Cluster Incl U92702 Early B-cell factor 3/cds = (73,1863)/gb = U92702/gi = 2114427/ug = Mm 30282/len = 3015/STRA = rev | 336.5 | P | 351.7 | P | 245.5 | P |
| 1 | 92193_r_at | Cluster Incl D12619 Proprotein convertase subtilisin/kexin type 5/cds = (69,2816)/gb = D12619/gi = 220565/ug = Mm.3401/ | 373.2 | P | 421.2 | P | 375.9 | P |
| | | len = 2994/STRA = for | | | | | | |
| 1 | 92211_at | Cluster Incl AF013969 Mus musculus antigen containing epitope to monoclonal antibody MMS-85/12 mRNA, partial cds/ | 601.8 | P | 850.2 | P | 400.6 | P |
| | | cds = (0,4704)/gb = AF013969/gi = 2384710/ug = Mm.34320/len = 4957/STRA = for | | | | | | |
| 1 | 92225_f_at | Cluster Incl U58280: Mus musculus second largest subunit of RNA polymerase I (RPA2) mRNA, complete cds/ | 681 | P | 686.7 | P | 523.9 | P |
| | | cds = (55,3462)/gb = U58280/gi = 1621593/ug = Mm 34589/len = 3978/STRA = for | | | | | | |
| 1 | 92294_at | Cluster Incl AW060793 UI-M-BH1-anu-g-02-0-UI s1 Mus musculus cDNA, 3 end/clone = UI-M-BH1-anu-g-02-0-UI/ | 351.1 | P | 375.2 | P | 272.7 | P |
| | | clone_end = 3/gb = AW060793/gi = 6008544/ug = Mm 3727/len = 453/STRA = rev | | | | | | |
| 1 | 92908_at | Cluster Incl L36829 Mus musculus alphaA-crystallin-binding protein 1 (alphaA-CRYBP1) gene/cds = (103,8169)/gb = L36829/ | 126.9 | P | 172.7 | P | 84.8 | P |
| | | gi = 818007/ug = Mm 4808/len = 8601/STRA = rev | | | | | | |
| 1 | 93144_at | Cluster Incl AI854602: UI-M-BH0-ake-c-05-0-UI s1 Mus musculus cDNA, 3 end/clone = UI-M-BH0-ake-c-05-0-UI/ | 721.7 | P | 807.9 | P | 716.5 | P |
| | | clone_end = 3/gb = AI854602/gi = 5498508/ug = Mm 5197/len = 481/STRA = for | | | | | | |
| 1 | 93693_at | Cluster Incl AI835373: UI-M-AQ0-aah-h-10-0-UI s1 Mus musculus cDNA, 3 end/clone = UI-M-AQ0-aah-h-10-0-UI/ | 479.9 | P | 607.3 | P | 479.8 | P |
| | | clone_end = 3/gb = AI835373/gi = 5469586/ug = Mm 844/len = 285/STRA = for | | | | | | |
| 1 | 94099_at | Cluster Incl AF049124 Mus musculus neuronal pentraxin 2 (Nptx2) mRNA, complete cds/cds = (67,1356)/gb = AF049124/ | 373.7 | P | 311.5 | P | 116.9 | P |
| | | gi = 2935463/ug = Mm 10099/len = 1608/STRA = for | | | | | | |
| 1 | 94125_at | Cluster Incl AF030454 Epithelial V-like antigen/cds = (174,821)/gb = AF030454/gi = 3169827/ug = Mm 10715/len = 1279/ | 373.4 | P | 388.5 | P | 356.5 | P |
| | | STRA = for | | | | | | |
| 1 | 94132_at | Cluster Incl X03920 M musculus GSHPx gene/cds = (37,642)/gb = X03920/gi = 51123/ug = Mm 1090/len = 923/STRA = for | 1653 | P | 1478 | P | 1418.2 | P |
| 1 | 94706_s_at | Cluster Incl AI327488 mb01a01 yt Mus musculus cDNA, 5 end/clone = IMAGE-318888/clone_end = 5/gb = AI327488/ | 732.5 | P | 996.9 | P | 764.2 | P |
| | | gi = 4061917/ug = Mm 13854/len = 420/STRA = for | | | | | | |
| 1 | 95326_at | Cluster Incl AF042180.Testis-specific protein, Y-encoded-like/cds = (55,1194)/gb = AF042180/gi = 3688779/ug = Mm 20445/ | 489.8 | P | 507.1 | P | 387 | P |
| | | len = 1455/STRA = for | | | | | | |
| 1 | 95351_at | Clusler Incl AA414339 vc66b04.s1 Mus musculus cDNA, 5 end/clone = IMAGE-779503/clone_end = 5/gb = AA414339/ | 138.5 | P | 112.5 | P | 71.7 | P |
| | | gi = 2074486/ug = Mm.21121/len = 411/STRA = for | | | | | | |
| 1 | 95400_l_at | Cluster Incl AI447783 mq84c02x1 Mus musculus cDNA, 3 end/clone = IMAGE-585410/clone_end = 3/gb = AI447783/ | 393.6 | P | 583 | P | 482.9 | P |
| | | gi = 4306405/ug = Mm 23936/len = 347/3TRA = rev | | | | | | |
| 1 | 95992_at | Cluster Incl C79950 C79950 Mus musculus cDNA, 3 end/clone = J0074C04/clone_end = 3/gb = C79950/gi = 2520280/ | 1097.8 | P | 1241.2 | P | 856.5 | P |
| | | ug = Mm 25056/len = 530/STRA = rev | | | | | | |
| 1 | 96546_r_at | Cluster Incl AI447510 mp69a04x1 Mus musculus cDNA, 3 end/clone = IMAGE-574446/clone_end = 3/gb = AI447510/ | 2200 | P | 2307.8 | P | 1990.8 | P |
| | | gi = 4304492/ug = Mm 28/len = 291/STRA = for | | | | | | |
| 1 | 96569_at | Cluster Incl AA268298 mz63a12 r1 Mus musculus cDNA, 5 end/clone = IMAGE-718078/clone_end = 5/gb = AA266298/ | 716.1 | P | 966.6 | P | 786.3 | P |
| | | gi = 1902420/ug = Mm 2972/len = 172/STRA = for | | | | | | |
| 1 | 97083_at | Cluster Incl AA600468.vm78h03 r1 Mus musculus cDNA, 5 end/clone = IMAGE-1004405/clone_end = 5/gb = AA600468/ | 571 | P | 559.2 | P | 536 | P |
| | | gi = 2434093/ug = Mm 31138/len = 283/STRA = for | | | | | | |
| 1 | 97163_g_at | Cluster Incl AI563699 vj83d06 y1 Mus musculus cDNA, 5 end/clone = lMAGE-935627/clone_end = 5/gb = AI563699/ | 754.9 | P | 746 | P | 721.2 | P |
| | | gi = 4522156/ug = Mm 35500/len = 392/STRA = rev | | | | | | |
| 1 | 97176_at | Cluster Incl AI645547: vt97b07.x1 Mus musculus cDNA, 3 end/clone = IMAGE-1179061/clone_end = 3/gb = AI645547/ | 867.1 | P | 962 | P | 957.3 | P |
| | | gi = 4724022/ug = Mm 35777/len = 493/STRA = rev | | | | | | |
| 1 | 97179_at | Clusler Incl C76543 C76543 Mus musculus cDNA, 3 end/clone = J0013B08/clone_end = 3/gb = C76543/gi = 2516873/ | 280.8 | P | 374.3 | P | 262 | P |
| | | ug = Mm 35801/len = 448/STRA = rev | | | | | | |
| 1 | 97723_at | Cluster Incl X62537 Chromobox homolog 2 (Drosophila Pc class)/cds = (147,1706)/gb = X62537/gi = 52976/ug = Mm 3908/ | 429.3 | P | 439.7 | P | 389.3 | P |
| | | len = 1872/STRA = for | | | | | | |
| 1 | 98344_f_at | Cluster Incl AI850438 UI-M-BG1-aih-a-04-0-UI s1 Mus musculus cDNA, 3 end/clone = UI-M-BG1-aih-a-04-0-UI/ | 1588.4 | P | 1457 | P | 1382.3 | P |
| | | clone_end = 3/gb = AI850438/gi = 5494344/ug = Mm 43257/len = 304/STRA = rev | | | | | | |
| 1 | 98778_at | Cluster Incl AI837543.UI-M-AL0-abs-d-04-0-UI s1 Mus musculus cDNA, 3 end/clone = UI-M-AL0-abs-d-04-0-UI/ | 293.9 | P | 294.1 | P | 289.3 | P |
| | | clone_end = 3/gb = AI837543/gi = 5471756/ug = Mm 4681/len = 246/STRA = for | | | | | | |
| 1 | 99389_at | Cluster Incl M21673: Mouse productively rearranged T-cell receptor-beta (LH8-1) gene (V5 2-D2.1-J2 5 region), complete | 207.9 | P | 300.2 | P | 230.7 | P |
| | | cds/cds = (0,347)/gb = M21673/gi = 201830/ug = Mm.56958/len = 348/STRA = for | | | | | | |
| 1 | 99847_at | Cluster Incl X73523 M musculus mRNA for Gal beta1, 3GalNAc alpha2,3-sialyltransferase/cds = (6,1019)/gb = X73523/ | 352.4 | P | 513.5 | P | 401 | P |
| | | gi = 402214/ug = Mm.6284/len = 1405/STRA = for | | | | | | |
| 1 | 100344_at | Cluster Incl AI464141 vc34b10 x1 Mus musculus cDNA, 3 end/clone = IMAGE-776443/clone_end = 3/gb = AI464141/ | 1196.7 | P | 1318.6 | P | 700.5 | P |
| | | gi = 4318171/ug = Mm 88145/len = 475/STRA = rev | | | | | | |
| 1 | 100387_g_at | Cluster Incl AB019600 Mus musculus mRNA for caspase9, complete cds/cds = (0,1364)/gb = AB019600/gi = 6440941/ | 529.3 | P | 583.2 | P | 359 | P |
| | | ug = Mm 88829/len = 1365/STRA = for | | | | | | |
| 1 | 101344_at | Cluster Incl AF019371 Cholecystokinin B receptor/cds = (0,1361)/gb = AF019371/gi = 2460176/ug = Mm.57011/len = 1362/ | 987.3 | P | 823.9 | P | 770.3 | P |
| | | STRA = for | | | | | | |
| 1 | 96464_at | Cluster Incl N28179 MDB1515 Mus musculus cDNA, 3 end/clone_end = 3/gb = N28179/gi = 1146035/ug = Mm.556/len = 284/ | 990.7 | P | 1078.9 | P | 880.2 | P |
| | | STRA = rev | | | | | | |
| 1 | 97004_at | Cluster Incl AJ133429.Mus musculus or17 gene/cds = (0,938)/gb = AJ133429/gi = 5869924/ug = Mm 88846/len = 939/ | 1804.9 | P | 1557.7 | P | 1398.2 | P |
| | | STRA = for | | | | | | |
| 1 | 97655_at | Cluster Incl Y11169 Desmocollin 3/cds = UNKNOWN/gb = Y11169/gi = 2765094/ug = Mm 89935/len = 2903/STRA = for | 200.4 | P | 364.5 | P | 134 | P |
| 1 | 97666_r_at | Cluster Incl C76213: C76213 Mus musculus cDNA, 3 end/clone = J0006A04/clone_end = 3/gb = C76213/gi = 2516543/ | 239.5 | P | 218.1 | P | 99.3 | P |
| | | ug = Mm.89955/len = 605/STRA = rev | | | | | | |
| 1 | 100717_at | Cluster Incl U90926: Mus musculus putative TNF-resistance related protein mRNA, complete cds/cds = (136,492)/ | 699.5 | P | 731.6 | P | 565.7 | P |
| | | gb = U90926/gi = 1906634/ug = Mm 2548/len = 540/STRA = for | | | | | | |
| 1 | 101226_at | Cluster Incl C77776 C77776 Mus musculus cDNA, 3 end/clone = J0037D08/clone_end = 3/gb = C77776/gi = 2518106/ | 932.3 | P | 945 | P | 866.4 | P |
| | | ug = Mm 5650/len = 622/STRA = rev | | | | | | |
| 1 | 102478_f_at | Cluster Incl AW122990 UI-M-BH2 1-apb-b-09-0-UI.s1 Mus musculus cDNA, 3 end/clone = UI-M-BH2 1-apb-b-09-0-UI/ | 440.7 | P | 416.2 | P | 313.5 | P |
| | | clone_end = 3/gb = AW122990/gi = 6098520/ug = Mm 48699/len = 387/STRA = rev | | | | | | |
| 1 | 94279_at | Cluster Incl AW125279 UI-M-BH2 1-apy-a-05-0-UI s1 Mus musculus cDNA, 3 end/clone = UI-M-BH2 1-apy-a-05-0-UI/ | 180.6 | P | 264.9 | P | 202.8 | P |
| | | clone_end = 3/gb = AW125279/gi = 6100809/ug = Mm 22514/len = 303/STRA = for | | | | | | |
| 1 | 94886_at | Cluster Incl L18888 Calnexin/cds = (0,1775)/gb = L18888/gi = 309131/ug = Mm 23929/len = 1776/STRA = rev | 528.1 | P | 477.4 | P | 589.6 | P |
| 1 | 95486_at | Cluster Incl AA794134.vr42h04 s1 Mus musculus cDNA, 5 end/clone = IMAGE-1123351/clone_end = 5/gb = AA794134/ | 463.6 | P | 568.9 | P | 514.3 | P |
| | | gi = 2857089/ug = Mm 24997/len = 155/STRA = for | | | | | | |
| 1 | 96023_at | Cluster Incl AI852472 UI-M-BHO-aip-f-10-0-UI s1 Mus musculus cDNA, 3 end/clone = UI-M-BH0-aip-l-10-0-UI/ | 140.5 | P | 181.5 | P | 179.4 | P |
| | | clone_end = 3/gb = AI852472/gi = 5496376/ug = Mm 25727/len = 402/STRA = rev | | | | | | |
| 1 | 96032_at | Cluster Incl L19737.ATP synthase, H + transporting, mitochondrial F0 complex, subunit c (subunit 9), isoform 1/ | 171.7 | P | 271.3 | P | 159.3 | P |
| | | cds = (3,413)/gb = L19737/gi = 309287/ug = Mm 258/len = 499/STRA = for | | | | | | |
| 1 | 96719_l_at | Cluster Incl X59382 Parvalbumin/cds = (56,388)/gb = X59382/gi = 509138/ug = Mm 2766/len = 615/STRA = for | 1540.5 | P | 1632.3 | P | 1366.6 | P |
| 1 | 97234_at | Cluster Incl AA792453: vs69d09 r1 Mus musculus cDNA, 5 end/clone = IMAGE-1151537/clone_end = 5/gb = AA792453/ | 177.9 | P | 281.9 | P | 205.2 | P |
| | | gi = 2855408/ug = Mm 27822/len = 351/STRA = for | | | | | | |
| 1 | 97508_at | Cluster Incl M29462.Malate dehydrogenase, soluble/cds = (81,1085)/gb = M29462/gi = 199094/ug = Mm 3156/len = 1288/ | 510.9 | P | 548.9 | P | 457.5 | P |
| | | STRA = for | | | | | | |
| 1 | 101981_at | Cluster Incl U09419 Mus musculus retinoid X receptor interacting protein (RIP15) mRNA, complete cds/cds = (130,1470)/ | 270.8 | P | 297.1 | P | 225.3 | P |
| | | gb = U09419/gi = 691713/ug = Mm. 968/len = 1841/STRA = for | | | | | | |
| 1 | 102804_at | Cluster Incl X67279 Biliary glycoprotein 1/cds = UNKNOWN/gb = X67279/gi = 50170/ug = Mm 14114/len = 2922/STRA = for | 1004.3 | P | 956.8 | P | 843.2 | P |
| 1 | 103013_at | Cluster Incl X77602 Upstream transcription factor 2/cds = (0,1028)/gb = X77602/gi = 609171/ug = Mm 15781/len = 1029/ | 878.3 | P | 745.9 | P | 695.4 | P |
| | | STRA = rev | | | | | | |
| 1 | 103843_at | Cluster Incl M36777 Guanine nucleotide binding protein, alpha o/cds = (21,1085)/gb = M36777/gi = 193359/ug = Mm.20964/ | 404.2 | P | 436.9 | P | 348.1 | P |
| | | len = 1910/STRA = for | | | | | | |
| 1 | 104629_at | Cluster Incl AI876546 uj60a09 x1 Mus musculus cDNA, 3 end/clone = IMAGE-1924312/clone_end = 3/gb = AI876546/ | 454.4 | A | 667 | P | 479 | P |
| | | gi = 5550595/ug = Mm 24331/len = 338/STRA = for | | | | | | |
| 1 | 95575_r_at | Cluster Incl AI641995 ub74e05x1 Mus musculus cDNA, 3 end/clone = IMAGE-1383468/clone_end = 3/gb = AI641995/ | 316.7 | P | 275.1 | P | 224.5 | P |
| | | gi = 4720470/ug = Mm 27821/len = 385/STRA = for | | | | | | |
| 1 | 95593_at | Cluster Incl AW125446 UI-M-BH2.3-aqh-h-05-0-UI s1 Mus musculus cDNA, 3 end/done = UI-M-BH2 3-aqh-h-05-0-UI/ | 982.8 | P | 1036.4 | P | 821.8 | P |
| | | clone_end = 3/gb = AW125446/gi = 6100976/ug = Mm 27902/len = 540/STRA = for | | | | | | |
| 1 | 95610_at | Cluster Incl AA636547 vq76h11 s1 Mus musculus cDNA, 5 end/clone = IMAGE-1108293/clone_end = 5/gb = AA636547/ | 176.5 | P | 128.8 | P | 165 | P |
| | | gi = 2560326/ug = Mm 27989/len = 460/STRA = for | | | | | | |
| 1 | 96126_at | Cluster Incl AF036894 Sphingosine phosphate lyase 1/cds = (60,1766)/gb = AF036894/gi = 2906010/ug = Mm.28119/ | 528.7 | P | 561.5 | P | 381.6 | A |
| | | len = 1911/STRA = for | | | | | | |
| 1 | 96790_f_at | Cluster Incl AW125222: UI-M-BH2.1-aps-c-12-0-UI s1 Mus musculus cDNA, 3 end/clone = UI-M-BH2.1-aps-c-12-0-UI/ | 391 | P | 401.4 | A | 341.6 | P |
| | | clone_end = 3/gb = AW125222/gi = 6100752/ug = Mm 29098/len = 396/STRA = for | | | | | | |
| 1 | 97437_f_at | Cluster Incl AW122483: UI-M-BH2.2-aou-f-01-0-UI.s1 Mus musculus cDNA, 3 end/clone = UI-M-BH2.2-aou-f-01-0-UI/ | 117.3 | P | 107.4 | P | 128.6 | P |
| | | clone_end = 3/gb = AW122483/gi = 6098005/ug = Mm30115/len = 277/STRA = for | | | | | | |
| 1 | 99528_at | Cluster Incl AW122015.UI-M-BH2 3-aoj-h-12-0-UI s1 Mus musculus cDNA, 3 end/clone = UI-M-8H2 3-aoj-h-12-0-UI/ | 205.3 | P | 206.1 | P | 181.1 | P |
| | | clone_end = 3/gb = AW122015/gi = 6097421/ug = Mm 42151/len = 405/STRA = for | | | | | | |
| 1 | 100941_at | Cluster Incl AF039567 Mus musculus Msx-interacting-zinc finger protein 1 (Miz1) mRNA, complete cds/cds = (26,1498)/ | 229.3 | P | 214.1 | P | 116 | P |
| | | gb = AF039567/gi = 2773147/ug = Mm 6370/len = 1847/STRA = for | | | | | | |
| 1 | 102251_at | Cluster Incl AF047714 Mus musculus melastatin mRNA, complete cds/cds = (307,1935)/gb = AF047714/gi = 3047241/ | 603.6 | P | 814.8 | P | 507.6 | P |
| | | ug = Mm.10752/len = 2722/STRA = for | | | | | | |
| 1 | 102681_at | Cluster Incl M24377 Early growth response 2/cds = (0,1262)/gb = M24377/gi = 198599/ug = Mm 1353/len = 1263/STRA = for | 1049.7 | P | 884.2 | P | 447.2 | M |
| 1 | 103706_at | Cluster Incl AI036406 ub65e10 r1 Mus musculus cDNA, 5 end/clone = IMAGE-1382634/clone_end = 5/gb = AI036406/ | 308.1 | P | 312.7 | P | 251.4 | P |
| | | gi = 3260097/ug = Mm 24078/len = 380/STRA = for | | | | | | |
| 1 | 104245_at | Cluster Incl AW124639 UI-M-BH2 1-apw-c-08-0-UI s1 Mus musculus cDNA, 3 end/clone = UI-M-BH2.1-apwc-08-0-UI/ | 1193 | P | 1394.4 | P | 1105.8 | P |
| | | clone_end = 3/gb = AW124839/gi = 6100169/ug = Mm28288/len = 413/STRA = rev | | | | | | |
| 1 | 104463_at | Cluster Incl AI157789 ue58d04 r1 Mus musculus cDNA, 5 end/clone = IMAGE-1495303/clone_end = 5/gb = AI157789/ | 360.2 | P | 592.1 | A | 267.2 | P |
| | | gi = 3686258/ug = Mm 29631/len = 443/STRA = for | | | | | | |
| 1 | 104503_at | Cluster Incl AI563623 vx92d09.x1 Mus musculus cDNA, 3 end/clone = IMAGE-1282673/clone_end = 3/gb = AI563623/ | 175.6 | P | 214.5 | P | 165.2 | P |
| | | gi = 4522080/ug = Mm 30648/len = 482/STRA = for | | | | | | |
| 1 | 92461_at | Cluster IncL AB021224-Mus musculus mRNA for membrane-type-4 matrix metalloproteinase, complete cds/cds = (85,1848)/ | 617.9 | P | 612.8 | A | 549.8 | P |
| | | gb = AB021224/gi = 5688930/ug = Mm 42047/len = 3490/STRA = for | | | | | | |
| 1 | 93872_at | Cluster Incl AF014117 Mus musculus GDNF receptor alpha (GDNFR-alpha) mRNA, complete cds/cds = (913,2319)/ | 60.5 | P | 73.7 | P | 81.4 | P |
| | | gb = AF014117/gi = 2624960/ug = Mm.88367/len = 2549/STRA = for | | | | | | |
| 1 | 94095_at | Cluster lncl AI552528 vx23a11.x1 Mus musculus cDNA, 3 end/clone = IMAGE-1265276/clone_end = 3/gb = AI552528/ | 320.8 | P | 388.8 | P | 257.7 | P |
| | | gi = 4484891/ug = Mm 100313/len = 496/STRA = rev | | | | | | |
| 1 | 95891_at | Cluster lncl AI591977.mt35c08 y1 Mus musculus cDNA, 5 end/clone = IMAGE-623054/clone_end = 5/gb = AI591977/ | 338.7. | P | 364.6 | P | 355.9 | P |
| | | gi = 4601025/ug = Mm 24058/len = 354/STRA = rev | | | | | | |
| 1 | 97726_at | Cluster Incl L35029 Glutamate receptor, ionotropic, NMDA2C (epsilon 3)/cds = (112,3723)/gb = L35029/gi = 602752/ | 690.3 | P | 897.6 | P | 674.7 | P |
| | | ug = Mm 39090/len = 3999/STRA = for | | | | | | |
| 1 | 98771_at | Cluster Incl L25890 Eph receptor B2/cds = (92,2983)/gb = L25890/gi = 414593/ug = Mm.4652/len = 3104/STRA = for | 344.1 | P | 347.3 | P | 339.3 | P |
| 1 | 98853_at | Cluster Incl D30779: Mouse mRNA for phospholipase A2 receptor, complete cds/cds = (131,4594)/gb = D30779/gi = 1375042/ | 185.1 | P | 220.7 | P | 202.3 | P |
| | | ug = Mm.5092/len = 5569/STRA = for | | | | | | |
| 1 | 99415_at | Cluster Incl U43321: Frizzled homolog 8, (Drosophila)/cds = (0,2057)/gb = U43321/gi = 1151259/ug = Mm.57055/len = 2058/ | 126.8 | P | 301.2 | P | 148.2 | P |
| | | STRA = for | | | | | | |
| 1 | 100295_at | Cluster Incl M34597 Mouse lg germline lambda-chain gene Vx-J2-C2-region, complete cds, clone 30X2/cds = (0,368)/ | 670.6 | P | 684.4 | P | 559.5 | P |
| | | gb = M34597/gi = 197929/ug = Mm.86392/len = 369/STRA = for | | | | | | |
| 1 | 94623_at | Cluster Incl U82698: Mlls musculus enameiln mRNA, complete cds/cds = (300,4124)/gb = U82698/gi = 2737960/ | 293.5 | P | 315 | P | 233.1 | P |
| | | ug = Mm 8014/len = 4170/STRA = for | | | | | | |
| 1 | 95848_at | Cluster Incl C76063: C76063 Mus musculus cDNA, 3 end/clone = J0003A08/clone_end = 3/gb = C76063/gi = 2516393/ | 317.2 | P | 378.6 | P | 223.6 | M |
| | | ug = Mm 24888/len = 626/STRA = rev | | | | | | |
| 1 | 96996_at | Cluster Incl AJ245706 Mus musculus mRNA for nicotinic acetylcholine receptor subunit alpha6 (NICA6 gene)/ | 274.7 | P | 336.6 | P | 214.5 | P |
| | | cds = (80,1564)/gb = AJ245706/gi = 5777608/ug = Mm 88637/len = 1595/STRA = for | | | | | | |
| 1 | 101595_at | Cluster Incl C78787 C78787 Mus musculus cDNA, 3 end/clone = J0055A07/clone_end = 3/gb = C78787/gi = 2979447/ | 212.2 | P | 298.1 | P | 207.2 | P |
| | | ug = Mm 5751/len = 387/STRA = rev | | | | | | |
| 1 | 96310_at | Cluster Incl L07508 Mouse Golli-mpb mRNA, complete cds (alternate transcript from clone BG21)/cds = (274,861)/ | 240.1 | P | 257.9 | P | 241.7 | P |
| | | gb = L07508/gi = 193586/ug = Mm 2992/len = 1407/STRA = for | | | | | | |
| 1 | 103062_at | Cluster Incl AB004664.Mus musculus DNA for Rab33B/cds = (316,1005)/gb = AB001664/gi = 2516240/ug = Mm 1664/ | 273.2 | P | 281.1 | P | 199.6 | P |
| | | len = 3421/STRA = for | | | | | | |
| 1 | 99497_at | Cluster Incl AI852332 UI-M-BHO-ajo-a-06-O-UI a1 Mus musculus cDNA, 3 end/clone = UI-M-BHO-ajo-a-06-0-UI/ | 623.8 | P | 659.8 | P | 613.6 | P |
| | | clone_end = 3/gb = AI852332/gi = 5496238/ug = Mm 41637/len = 501/STRA = for | | | | | | |
| 1 | 99954_at | Cluster Incl AW046474 UI-M-BH1-akz-a-08-0-UI s1 Mus musculus cDNA, 3 end/clone = UI-M-BH1-akz-a-08-0-UI/ | 755 | P | 731 | P | 705.8 | P |
| | | clone_end = 3/gb = AW046474/gi = 5907003/ug = Mm 439/len = 499/STRA = for | | | | | | |
| 1 | 100405_at | Cluster Incl X56683 Mouse mRNA coding for modifier 2 protein/cds = (0,521)/gb = X56683/gi = 53164/ug = Mm 46536/ | 252.8 | P | 354.9 | P | 191.9 | P |
| | | len = 522/STRA = rev | | | | | | |
| 1 | 102231_at | Cluster Incl AB017614 Mus musculus mRNA for OASIS protein, complete cds/cds = (27,1550)/gb = AB017614/gi = 4519620/ | 1169.4 | P | 1337.8 | A | 1207.8 | P |
| | | ug = Mm.10353/len = 1908/STRA = rev | | | | | | |
| 1 | 102711_at | Cluster Incl U85055: Mus musculus rap1/rap2 interacting protein mRNA, complete cds/cds = (0,1643)/gb = U85055/ | 421.9 | P | 583.9 | P | 354.9 | P |
| | | gi = 1814395/ug = Mm 1426/len = 2208/STRA = rev | | | | | | |
| 1 | 103275_at | Cluster Incl U13836 Mus musculus vacuolar adenosine triphosphatase subunit Ac116 mRNA, complete cds/cds = (18,2537)/ | 234.3 | P | 263.8 | P | 191.8 | P |
| | | gb = U13836/gi = 3955095/ug = Mm.20869/len = 2557/STRA for | | | | | | |
| 1 | 103766_at | Cluster Incl X97817 Semaphorin F/cds = (27,3260)/gb = X97817/gi = 1419018/ug = Mm 24733/len = 3949/STRA = for | 87.1 | P | 89.7 | P | 56.1 | P |
| 1 | 93206_g_at | Cluster Incl M14167 Mouse ADA gene encoding adenosine deaminase, promoter region/cds = (142,144)/gb = M14167/ | 270.3 | P | 283.4 | P | 227.9 | M |
| | | gi = 191688/ug = Mm.56978/len = 145/STRA = for | | | | | | |
| 1 | 93211_at | Cluster Incl AF032115 Mus musculus cysteine string protein mRNA complete cds/cds = (0,596)/gb = AF032115/ | 574.1 | P | 565.2 | A | 432.7 | P |
| | | gi = 2642628/ug = Mm 57022/len = 597/STRA = for | | | | | | |
| 1 | 95295_s_at | Cluster Incl X59398.FMS-like tyrosine kinase 3/cds = (81,3083)/gb = X59398/gi = 50978/ug = Mm 194/len = 3521/STRA = for | 521.2 | P | 509.7 | P | 352.3 | P |
| 1 | 96557_at | Cluster Incl AB010833 Patched homolog 2/cds = (0,3548)/gb = AB010833/gi = 2842557/ug = Mm 28529/len = 3549/STRA = for | 470.9 | P | 600.5 | P | 441 | P |
| 1 | 97097_at | Cluster Incl AW125669: UI-M-BH2 2-aqi-c-12-0-UI S1 Mus musculus cDNA, 3 end/clone = UI-M-BH2 2-aqi-c-12-0-UI/ | 287.8 | P | 299.1 | P | 249.6 | P |
| | | clone_end = 3/gb = AW125669/gi = 6101199/ug = Mm 3183/len = 507/STRA = for | | | | | | |
| 1 | 97136_at | Cluster IncI AA571388 vl77a12 s1 Mus musculus cDNA, 3 end/clone = IMAGE-978238/clone_end = 3/gb = AA571388/ | 89.9 | P | 39 | P | 1.3 | P |
| | | gi = 2346317/ug = Mm 34930/len = 440/STRA = for | | | | | | |
| 1 | 98349_at | Cluster Incl X62646 M musculus mRNA for IL-6 signal transducer (gp130)/cds = (88,2841)/gb = X62646/gi = 840816/ | 102.3 | P | 202.7 | P | 34.5 | P |
| | | ug = Mm 4364/len = 2995/STRA = for | | | | | | |
| 1 | 100750_at | Cluster IncI AA414903 vc50b09.r1 Mus musculus cDNA, 3 end/clone = IMAGE-777977/clone_end = 3/gb = AA414903/ | 236 | P | 291.1 | P | 231 | P |
| | | gi = 2075133/ug = Mm 3808/len = 151/STRA = rev | | | | | | |
| 1 | 101661_r_at | Cluster Incl AA420337 vc51dO3 r1 Mus musculus cDNA, 3 end/clone = IMAGE-778085/clone_end = 3/gb = AA420337/ | 55.4 | P | 75.6 | P | 50.7 | P |
| | | gi = 2080894/ug = Mm.6761/len = 281/STRA = rev | | | | | | |
| 1 | 94072_g_at | Cluster Incl AI847904 UI-M-AI1-afl-a-09-0-UI.s1 Mus musculus cDNA, 3 end/clone = UI-M-AI1-afl-o-09-0-UI/clone_end = 3/ | 698 | A | 778.1 | A | 572.2 | P |
| | | gb = AI847904/gi = 5491810/ug = Mm 22114/len = 350/STRA = for | | | | | | |
| 1 | 97222_at | Cluster Incl AI845921 UI-M-AK1-aex-d-11-0-UI s1 Mus musculus cDNA, 3 end/clone = UI-M-AK1-aea-d-11-0-UI/ | 517.7 | P | 711.7 | P | 611.1 | A |
| | | clone_end = 3/gb = AI845921/gi = 5489827/ug = Mm.27756/len = 298/STRA = for | | | | | | |
| 1 | 97821_at | Cluster Incl AI646056 vw54c11.x1 Mus musculus cDNA, 3 end/clone = IMAGE-1247636/clone_end = 3/gb = AI646056/ | 569.3 | P | 552.6 | A | 549.6 | P |
| | | gi = 4724531/ug = Mm 28202/len = 561/STRA = rev | | | | | | |
| 1 | 100619_r_at | Cluster Incl AA062013 mj83h01 r1 Mus musculus cDNA, 5 end/clone = IMAGE-482737/clone_end = 5/gb = AA062013/ | 331.3 | M | 563.5 | P | 315.6 | M |
| | | gi = 1555829/ug = Mm 658/len = 529/STRA = for | | | | | | |
| 1 | 95545_at | Cluster Incl X04480.lnsulin-like growth factor 1/cds = (152,535)/gb = X04480/gi = 51801/ug = Mm 2770/len = 696/STRA = for | 447.2 | P | 775.9 | P | 560.5 | P |
| 1 | 98443_at | Cluster Incl AI882416 uc01c09 r1 Mus musculus cDNA, 5 end/clone = IMAGE-1396720/clone_end = 5/gb = AI882416/ | 645.6 | A | 685.8 | P | 592.7 | P |
| | | gi = 5567505/ug = Mm 34519/len = 338/STRA = for | | | | | | |
| 1 | 99463_at | Cluster Incl X63023 Cytochrome P450, steroid Inducible 3a13/cds = (83,1594)/gb = X63023/gi = 50634/ug = Mm 4094/ | 133.7 | P | 155.5 | P | 102.7 | P |
| | | len = 1881/STRA = for | | | | | | |
| 1 | 100020_at | Cluster Incl J04036 Solute carrier family 4 (anlon exchanger), member 2/cds = (182,3895)/gb = J04036/gi = 192132/ | 391.6 | P | 497.3 | P | 390.8 | P |
| | | ug = Mm 4580/len = 4088/STRA = for | | | | | | |
| 1 | 101417_at | Cluster Incl AF020191: Mus musculus proline-rich protein 13 mRNA, partial cds/cds = (0,511)/gb = AF020191/gi = 2501952/ | 353.7 | P | 329 | P | 296.6 | P |
| | | ug = Mm.7412/len = 1370/STRA = for | | | | | | |
| 1 | 101840_at | Cluster Incl L06864 Mus musculus epidermal growth factor receptor (EGFR) mRNA, partial cds/cds = (0,363)/gb = L06864/ | 197.4 | P | 242.7 | P | 151.3 | P |
| | | gi = 193001/ug = Mm 8534/len = 364/STRA = for | | | | | | |
| 1 | 102242_at | Cluster Incl AF050182 Mus musculus PERIOD 3 (Per3) mRNA, complete cds/cds = (357,3698)/gb = AF050182/gi = 3136149/ | 552.2 | M | 774 | P | 655.5 | P |
| | | ug = Mm 10723/len = 4863/STRA = for | | | | | | |
| 1 | 104238_at | Cluster Incl U60881: ADP-ribosyltransferase 5/cds = (382,1311)/gb = U60881/gi = 1522688/ug = Mm.2816/len = 1547/ | 335.2 | P | 365.2 | M | 287.8 | P |
| | | STRA = for | | | | | | |
| 1 | 92745_at | Cluster Incl AB005458 Homeo box A9/cds = (446,763)/gb = AB005458/gi = 3080549/ug = Mm 4694/len = 764/STRA = for | 81.1 | A | 137.9 | P | 104.1 | P |
| 1 | 93689_at | Cluster Incl AW122071 UI-M-BH2 2-aoo-e-12-0-UI s1 Mus musculus cDNA, 3 end/clone = UI-M-BH2 2-aoo-e-12-0-UI/ | 741.6 | P | 662.5 | A | 678.9 | P |
| | | clone_end = 3/gb = AW122071/gi = 6097478/ug = Mm 840/len = 413/STRA = for | | | | | | |
| 1 | 94135_at | Cluster Incl M76987 Transcription factor CP2/cds = UNKNOWN/gb = M76987/gi = 191802/ug = Mm 1139/len = 4023/ | 473.2 | P | 441.8 | A | 388 | M |
| | | STRA = for | | | | | | |
| 1 | 94171_at | Cluster Incl AF051367 Mus musculus pactolus mRNA, complete cds/cds = (63,2279)/gb = AF051367/gi = 3287490/ | 318.6 | P | 322.2 | P | 300.9 | P |
| | | ug = Mm 12872/len = 2628/STRA = for | | | | | | |
| 1 | 97096_at | Cluster Incl J02935 Mouse cAMP-dependent protein kinase type II regulatory subunit mRNA, 3 end/cds = (0,1205)/ | 667.3 | A | 636.6 | P | 547.3 | A |
| | | gb = J02935/gi = 200356/ug = Mm 3176/len = 1533/STRA = for | | | | | | |
| 1 | 98867_at | Cluster Incl AI848793 UI-M-AJ1-ahg-d-12-0-UI s1 Mus musculus cDNA, 3 end/clone = UI-M-AJ1-ahg-d-12-0-UI/ | 171.8 | P | 200.1 | P | 181.3 | P |
| | | clone_end = 3/gb = AI848793/gi = 5492699/ug = Mm 5152/len = 388/STRA = rev | | | | | | |
| 1 | 99810_at | Cluster Incl X91864 Glutathione peroxidase 2, pseudogene 1/cds = (0,158)/gb = X91864/gi = 1359980/ug = Mm 57225/ | 466.2 | A | 447.3 | P | 227.9 | P |
| | | len = 159/STRA = for | | | | | | |
| 1 | 102557_at | Cluster Incl U62673 Mus musculus histone H2a(A)-613, histone H2a(B)-613, and histone H2b-613 (H2b) genes, complete | 110.4 | M | 124.4 | M | 95.2 | P |
| | | cds/cds = (0,392)/gb = U62673/gi = 1458138/ug = Mm 57213/len = 393/STRA = for | | | | | | |
| 1 | 94551_at | Cluster Incl AI853061 UI-M-BH0-ajn-g-12-0-UI s1 Mus musculus cDNA, 3 end/clone = UI-M-BH0-ajn-g-12-0-UI/ | 339.1 | P | 310.2 | A | 253.7 | A |
| | | clone_end = 3/gb = AI853061/gi = 5496967/ug = Mm 29087/len = 363/STRA = for | | | | | | |
| 1 | 94556_at | Cluster Incl AI746846 uI08b08.y1 Mus musculus cDNA, 5 end/clone = IMAGE-2076183/clone_end = 5/gb = AI746846/ | 156.4 | A | 193 | P | 111.4 | A |
| | | gi = 5125110/ug = Mm 29101/len = 596/STRA = for | | | | | | |
| 1 | 102372_at | Cluster Incl M90766 Mouse Ig active joining chain (j chain) mRNA of the b allele, complete cds/cds = (21,500)/gb = M90766/ | 241.5 | M | 233.8 | A | 147.2 | P |
| | | gi = 194473/ug = Mm.1192/len = 501/STRA = for | | | | | | |
| 1 | 103614_at | Cluster Incl AW047899 UI-M-BH1-ain-a-06-0-UI s1 Mus musculus cDNA, 3 end/clone = UI-M-BH1-aln-a-06-0-UI/ | 152.2 | P | 205.3 | P | 193.1 | P |
| | | clone_end = 3/gb = AW047B99/gi = 5908428/ug = Mm 20225/len = 403/STRA = rev | | | | | | |
| 1 | 103840_at | Cluster Incl AJ01,1923 Mus musculus mRNA for cell cycle checkpoint protein/cds = (75,2138)/gb = AJ011923/gi = 3758877/ | 133.6 | P | 330.3 | P | 186.2 | P |
| | | ug = Mm 20947/len = 2640/STRA = for | | | | | | |
| 1 | 104036_at | Cluster Incl AI845447 UI-M-A01-aej-f-01-0-UI s1 Mus musculus cDNA, 3 end/clone = UI-M-AO1-aej-f-01-0-UI/ | 256 | P | 282.9 | P | 106.9 | P |
| | | clone_end = 3/gb = AI845447/gi = 5489353/ug = Mm 21440/len = 343/STRA = for | | | | | | |
| 1 | 96806_at | Cluster Incl AI843802 UI-M-AN1-afc-e-12-0-UI.s1 Mus musculus cDNA, 3 end/clone = UI-M-AN1-afc-e-12-0-UI/ | 425.6 | A | 463 | P | 395.4 | A |
| | | clone_end = 3/gb = AI843802/gi = 5478015/ug = Mm 29209/len = 537/STRA = for | | | | | | |
| 1 | 98457_at | Cluster Incl AF020195 Mus musculus pancreas sodium bicarbonate cotransporter mRNA, complete cds/cds = (29,3268)/ | 141.9 | M | 161.9 | P | 135.6 | P |
| | | gb = AF020195/gi = 3298571/ug = Mm.34747/len = 3337/STRA = for | | | | | | |
| 1 | 102265_at | Cluster Incl X59060 Myogenic factor 6/cds = (25,753)/gb = X59060/gi = 53297/ug = Mm 11/len = 1258/STRA = for | 400.1 | P | 540.6 | P | 270.2 | P |
| 1 | 102891_at | Cluster Incl D86527: Werner syndrome homolog (human)/cds = (183,4388)/gb = D86527/gi = 2130974/ug = Mm 15446/ | 279.5 | P | 317 | P | 287.6 | A |
| | | len = 4955/STRA = rev | | | | | | |
| 1 | 104273_at | Cluster Incl U95215 Bile acid CoA-amino acid n-acyltransferase/cds = (189,1451)/gb = U95215/gi = 2130557/ug = Mm.2859/ | 277.6 | P | 279.4 | M | 225.3 | A |
| | | len = 1870/STRA = rev | | | | | | |
| 1 | 92895_at | Cluster Incl AF038857 untitled/cds = (0,2297)/gb = AF038857/gi = 2952265/ug = Mm.4774/len = 2298/STRA = for | 213 | A | 171.1 | P | 170.9 | M |
| 1 | 93185_at | Cluster Incl AW045808 UI-M-BH1-akt-h-04-0-UI.s1 Mus musculus cDNA, 3 end/clone = UI-M-BH1-akt-h-04-0-UI/ | 190.2 | P | 318.8 | P | 224.9 | A |
| | | clone_end = 3/gb = AW0458O8/gi = 5906337/ug = Mm 5473/len = 367/STRA = rev | | | | | | |
| 1 | 94762_at | Cluster Incl U92972 Mus musculus protease-activated receptor 3 (PAR3) mRNA, complete cds/cds = (152,1261)/ | 367.9 | P | 508.2 | A | 259.2 | M |
| | | gb = U92972/gi = 1938376/ug = Mm 1612/len = 2409/STRA = rev | | | | | | |
| 1 | 102580_r_at | Cluster Incl M11988 Homeo box A6/cds = (0,293)/gb = M11988/gi = Mm.57244/len = 294/STRA = for | 382.4 | A | 326.5 | P | 215.8 | P |
| 1 | 101233_at | Cluster Incl C78616.C78616 Mus musculus cDNA, 3 end/clone = J0052A12/clone_end = 3/gb = C78616/gi = 2518946/ | 56.5 | P | 77.3 | P | 64.7 | A |
| | | ug = Mm 5725/len = 541/STRA = rev | | | | | | |
| 1 | 101687_r_at | Cluster Incl C79518.C79518 Mus musculus cDNA,3 end/clone = J0067E04/clone_end = 3/gb = C79518/gi = 2519848/ | 99.6 | P | 136.6 | P | 114.4 | P |
| | | ug = Mm 36431/len = 252/STRA = rev | | | | | | |
| 1 | AFFX-BioDn- | E. Coli BioD 3 prime | 207.2 | P | 266.2 | P | 177.1 | A |
| | 3_at | | | | | | | |
| 1 | 93788_at | Cluster Incl M37831.Cytochrome C oxidase, subunit IV/cds = (1,510)/gb = M37831/gi = 459880/ug = Mm 2136/len = 610/ | 176.9 | M | 174.3 | P | 161.8 | A |
| | | STRA = for | | | | | | |
| 1 | 94235_at | Cluster Incl AW050034 UI-M-BH1-amm-b-10-0-UI s1 Mus musculus cDNA, 3 end/clone = UI-M-BH1-amm-b-10-0-UI/ | 98.5 | P | 84.9 | A | 95.1 | P |
| | | clone_end = 3/gb = AW050034/gi = 5910563/ug = Mm.22327/len = 531/STRA = for | | | | | | |
| 1 | 96044_at | Cluster Incl AF067660: Bcl2-like 10/cds = (142,717)/gb = AF067660/gi = 3955265/ug = Mm 25988/len = 1225/STRA = for | 372.2 | M | 491 | P | 234.8 | A |
| 1 | 98526_r_at | Cluster Incl AI854606 UI-M-BH0-ake-c-09-0-UI s1 Mus musculus cDNA, 3 end/clone = UI-M-BH0-ake-c-09-0-UI/ | 332.2 | P | 467.7 | P | 267.9 | P |
| | | clone_end = 3/gb = AI854606/gi = 5498512/ug = Mm 35829/len = 472/STRA = rev | | | | | | |
| 1 | 102765_at | Cluster Incl AF071317-Mus musculus COP9 complex subunit 7b (COPS7b) mRNA, complete cds/cds = (130,924)/ | 299.5 | M | 538 | P | 348.8 | A |
| | | gb = AF071317/gi = 3309175/ug = Mm 12973/len = 1990/STRA = for | | | | | | |
| 1 | 102771_at | Cluster Incl AF091628 Mus musculus ERG-associated protein ESET mRNA, complete cds/cds = (101,4024)/gb = AF091628/ | 434.6 | A | 453 | A | 389.5 | P |
| | | gi = 3644041/ug = Mm 13162/len = 4602/STRA = rev | | | | | | |
| 1 | 102861_at | Cluster Incl AF028739 Imprinted polyspecific membrane transporter-like gene 1/cds = (179,1399)/gb = AF02B739/ | 213.3 | P | 274 | P | 275.3 | P |
| | | gi-2583224/ug = Mm 15093/len = 1466/STRA = for | | | | | | |
| 1 | 96184_at | Cluster Incl AIB35242.UI-M-AQ0-aaa-h-10-0-UI s1 Mus musculus cDNA, 3 end/clone = UI-M-AQ0-aaa-h-10-0-UI/ | 894.5 | P | 937.9 | A | 708.6 | P |
| | | clone_end = 3/gb = AI835242/gi = 5489455/ug = Mm 28462/len = 294/STRA = for | | | | | | |
| 1 | 98970_at | Cluster Incl AB016044 Mus musculus mRNA for GGPP synthase, complete cds/cds = (107,1009)/gb = AB016044/ | 97.5 | A | 154.6 | P | 88.7 | M |
| | | gi = 4586887/ug = Mm 36520/len = 1025/STRA = for | | | | | | |
| 1 | 100961_at | Cluster Incl AF012871.Long(electocardiographic) QT syndrome 2/cds = (321,3809)/gb = AF012871/gi = 2582014/ | 349.6 | P | 502 | A | 347.6 | A |
| | | ug = Mm 6539/len = 4221/STRA = for | | | | | | |
| 1 | 101399_at | Cluster Incl M77174 Perlecan (heparan sulfate proteoglycan 2)/cds = (578,11701)/gb = M77174/gi = 200295/ug = Mm 7257/ | 653.9 | M | 872.5 | P | 640.2 | P |
| | | len = 12667/STRA = for | | | | | | |
| 1 | 101942_at | Cluster Incl AA839050: vw47a01.r1 Mus musculus cDNA, 5 end/clone = IMAGE-1246920/clone_end = 5/gb = AA839050/ | 506.9 | A | 566.5 | A | 275.3 | A |
| | | gi = 2915145/ug = Mm.854/len = 511/STRA = for | | | | | | |
| 1 | 104239_at | Cluster Incl AW208513: uo60102.x1 Mus musculus cDNA, 3 end/clone = IMAGE-2646939/clone_end = 3/gb = AW208513/ | 98.5 | P | 88.2 | A | 63.3 | M |
| | | gi = 6514453/ug = Mm 28172/len = 461/STRA = for | | | | | | |
| 1 | 104500_at | Cluster Incl L34111: Alpha-L-iduronidase/cds = (26,1930)/gb = L34111/gi = 623169/ug = Mm 3054/len = 3109/STRA = for | 157.1 | A | 223.6 | P | 138.3 | A |
| 1 | 92271_at | Cluster Incl X63963 Paired box gene 6/cds = (162,1472)/gb = X63963/gi = 460050/ug = Mm 3608/len = 2481/STRA = for | 701.3 | P | 683.9 | P | 663.9 | P |
| 1 | 92458_at | Cluster Incl AJ003133 Mus musculus mRNA for ORC1-related protein, complete cds/cds = (123,2645)/gb = AJ003133/ | 291.4 | A | 210 | P | 196.3 | M |
| | | gi = 4034784/ug = Mm 42024/len = 3017/STRA = for | | | | | | |
| 1 | 94177_at | Cluster Incl Y15733 Mus musculus mRNA for 17-beta-hydroxysteroid dehydrogenase type 7/cds = (03,1067)/gb = Y15733 | 87.5 | A | 143.1 | P | 183.9 | P |
| | | gi = 3319970/ug = Mm 12882/len = 1756/STRA = for | | | | | | |
| 1 | 96593_at | Cluster Incl X87257: ELK1, member of ETS oncogene family/cds = (473,1762)/gb = X87257/gi = 836634/ug = Mm 3064/ | 152.1 | M | 193.7 | P | 142. 9 | P |
| | | len = 2286/STRA = for | | | | | | |
| 1 | 102078_at | Cluster Incl AF066075 untitled/cds = (127,474)/gb = AF066075/gi = 4092929/ug = Mm 42082/len = 714/STRA = for | 31.7 | P | 52.6 | P | 49.7 | A |
| 1 | 94541_at | Cluster Incl AI839549 UI-M-AN0-acn-h-08-0-UI.s1 Mus musculus cDNA, 3 end/clone = UI-M-AN0-acn-h-08-0-UI/ | 161.7 | A | 275 | P | 198.1 | M |
| | | clone_end = 3/gb = AI839549/gi = 5473762/ug = Mm 29065/len = 162/STRA = for | | | | | | |
| 1 | 101513_at | Cluster Incl AI840454 UI-M-AN0-aci-c-01-o-UI s1 Mus musculus cDNA, 3 end/clone = UI-M-AN0-aci-c-01-o-UI/ | 181.3 | A | 190.6 | P | 154.9 | P |
| | | clone_end = 3/gb = AI840454/gi = 5474667/ug = Mm.836/len = 446/STRA = for | | | | | | |
| 1 | 102995_s_at | Cluster Incl M13226 Granzyme A/cds = (198,971)/gb = M13226/gi = 201048/ug = Mm.15510/len = 1039/STRA = for | 265.7 | A | 314.6 | P | 196.7 | A |
| 1 | 103361_at | Cluster Incl AI853856 UI-M-BH0-ajr-e-10-0-UI.s1 Mus musculus cDNA, 3 end/clone = UI-M-BH0-ajr-e-10-0-UI/ | 68.1 | M | 56 | P | 45.8 | A |
| | | clone_end = 3/gb = AI853856/gi = 5497762/ug = Mm 18506/len = 374/STRA = rev | | | | | | |
| 1 | 104581_at | Cluster Incl AI845438 UI-M-AO1-aej-d-10-0-UI s1 Mus musculus cDNA, 3 end/clone = UI-M-AO1-aoj-d-10-0-UI/ | 32.3 | M | 67.3 | P | 35 | A |
| | | clone_end = 3/gb = AI845438/gi = 5489339/ug = Mm 23975/len = 369/STRA = for | | | | | | |
| 1 | 104708_at | Cluster Incl AI463490 vw62d08.x1 Mus musculus cDNA, 3 end/clone = IMAGE-1248399/clone_end = 3/gb = AI463490/ | 304.9 | P | 322.3 | A | 352.3 | A |
| | | gi = 4317520/ug = Mm 24408/len = 180/STRA = for | | | | | | |
| 1 | 102649_s_at | Cluster Incl D64162 Mouse clone Rae-123 mRNA for cell surface protein, complete cds/cds = (92,853)/gb = D84162/ | 171.3 | A | 232.5 | P | 131.8 | M |
| | | gi = 1311547/ug = Mm 1326/len = 1242/STRA = for | | | | | | |
| 1 | 104449_at | Cluster Incl X81202: Glycine receptor, beta subunit/cds = (0,1455)/gb = X81202/gi = 557654/ug = Mm 29422/len = 1465/ | 156 | A | 171.4 | P | 91 | P |
| | | STRA = for | | | | | | |
| 1 | 104509_at | Cluster Incl AF059213 Mus musculus cholesterol 25-hydroxylase mRNA, complete cds/cds = (9,905)/gb = AF059213/ | 197 | A | 194.3 | A | 157.9 | A |
| | | gi = 4038305/ug = Mm 30824/len = 1365/STRA = rev | | | | | | |
| 1 | 92312_at | Cluster Incl U55772 Mus musculus p170 phosphatidylinositol 3-kinase mRNA, complete cds/cds = (57,4586)/gb = U55772/ | 148.9 | A | 213.8 | P | 184.5 | A |
| | | gi = 1305537/ug = Mm 3810/len = 5067/STRA = for | | | | | | |
| 1 | 92493_at | Cluster Incl Y10926 M musculus mRNA for transcription factor RBP-L/cds = (103,1650)/gb = Y10926/gi = 2052118/ | 211.6 | P | 269.8 | A | 236.1 | A |
| | | ug = Mm 42177/len = 2456/STRA = for | | | | | | |
| 1 | 93452_at | Cluster Incl AF031896 Cerberus 1/cds = (82,900)/gb = AF031896/gi = 2654026/ug = Mm 6780/len = 1752/STRA = for | 177.6 | A | 213.2 | P | 162.8 | A |
| 1 | 97143_at | Cluster Incl C79450 C79450 Mus musculus cDNA, 3 end/clone = J0066E08/clone_end = 3/gb = C79450/gi = 2519780/ | 182.1 | A | 299.6 | M | 188.3 | A |
| | | ug = Mm 34943/len = 579/STRA = rev | | | | | | |
| 1 | 97156_at | Cluster Incl D32200 Mouse gene for Hes-3/cds = (0,527)/gb = D32200/gi = 699611/ug = Mm 3516/len = 528/STRA = for | 294.5 | P | 331.4 | P | 230.7 | A |
| 1 | 97701_at | Cluster Incl AI181906 ud73e11 x1 Mus musculus cDNA, 3 end/clone = IMAGE-1451564/clone_end = 3/gb = AI181906/ | 148.3 | A | 223.8 | P | 148.7 | A |
| | | gi = 3732544/ug = Mm 38004/len = 387/STRA = rev | | | | | | |
| 1 | 99893_at | Cluster Incl AF020737: Fibroblast growth factor 13/cds = (9,743)/gb = AF020737/gi = 2444476/ug = Mm 7995/len = 817/ | 88.3 | A | 105.9 | P | 76.4 | A |
| | | STRA = for | | | | | | |
| 1 | 102621_at | Cluster Incl AF090866 Mus musculus CDO (Cdo) mRNA, complete cds/cds = (29,3781)/gb = AF090866/gi = 3644025/ | 355 .8 | A | 385.8 | P | 362.1 | P |
| | | ug = Mm 20439/len = 3914/STRA = for | | | | | | |
| 1 | 97665_1_at | Cluster Incl C76213 C76213 Mus musculus cDNA, 3 end/clone = J0006A04/clone_end = 3/gb = C76213/gi = 2516543/ | 166.8 | A | 159.9 | P | −25.6 | P |
| | | ug = Mm 89955/len = 605/STRA = rev | | | | | | |
| 1 | 101614_at | Cluster Incl AA672848 vp57b02 r1 Mus musculus cDNA, 3 end/clone = IMAGE-1080747/clone_end = 3/gb = AA672648/ | 174.8 | A | 218.4 | P | 134.4 | P |
| | | gi = 2644865/ug = Mm 6126/len = 404/STRA = rev | | | | | | |
| 1 | 102143_at | Cluster Incl AW047018: UI-M-BH1-alp-h-06-0-UI s1 Mus musculus cDNA, 3 end/clone = UI-M-BH1-alp-h-06-0-UI/ | 304 | A | 316.9 | P | 358.6 | P |
| | | clone_end = 3/gb = AW047018/gi = 5907547/ug = Mm 44276/len = 396/STRA = rev | | | | | | |
| 2 | 92573_at | Cluster Incl AB021743 Mus musculus mRNA for PR65 (type 2A serine/threonine protein phosphatase A subunit), complete | 2437.3 | P | 2101.2 | P | 2210.8 | P |
| | | cds/cds = (0,1769)/gb = AB021743/gi = 4514638/ug = Mm. 1034/len = 1770/STRA = for | | | | | | |
| 2 | 92599_at | Cluster Incl AF029843 Mus musculus phosphoglycerate mutase muscle-specific subunit mRNA, complete cds/ | 1752.8 | P | 943.1 | P | 1490.8 | P |
| | | cds = (45,806)/gb = AF029843/gi = 3043914/ug = Mm 10738/len = 836/STRA = for | | | | | | |
| 2 | 92621_at | Cluster Incl X97982 M musculus mRNA for poly(C)-binding protein, splice variant E/cds = (221,1270)/gb = X97982/ | 3020.7 | P | 2444.4 | P | 2772.8 | P |
| | | gi = 1360002/ug = Mm.111/len = 1655/STRA = rev | | | | | | |
| 2 | 92637_at | Cluster Incl J03928 Phosphofructokinase, liver, B-type/cds = (42,2384)/gb = J03928/gi = 200297/ug = Mm 1166/len = 2688/ | 1081.5 | P | 887.8 | P | 81B.3 | P |
| | | STRA = for | | | | | | |
| 2 | 92775_at | Cluster Incl AA656757: vr50c03.s1 Mus musculus cDNA, 5 end/clone = IMAGE-1124068/clone_end = 5/gb = AA656757/ | 946.7 | P | 690 | P | 656 | P |
| | | gi = 2592911/ug = Mm 12267/len = 407/STRA = for | | | | | | |
| 2 | 92777_at | Cluster Incl M32490 Insulin-like growth factor binding protein 10/cds = (189,1328)/gb = M32490/gi = 192909/ug = Mm 1231/ | 736.1 | P | 150.6 | P | 180.4 | P |
| | | len = 2018/STRA = for | | | | | | |
| 2 | 92787_at | Cluster Incl AI845902: UI-M-AK1-aex-b-07-0-UI s1 Mus musculus cDNA, 3 end/clone = UI-M-AK1-aex-b-07-0-UI/ | 1232.8 | P | 396.2 | P | 862.9 | P |
| | | clone_end = 3/gb = AI845902/gi = 5489808/ug = Mm.12459/len = 358/STRA = for | | | | | | |
| 2 | 93104_at | Cluster Incl Z16410 M musculus btg1 mRNA/cds = (0,515)/gb = Z16410/gi = 50187/ug = Mm 16596/len = 516/STRA = for | 2182.9 | P | 1736.3 | P | 1798.8 | P |
| 2 | 93285_at | Cluster Incl AI845584 UI-M-AQ1-adx-c-06-0-UI s1 Mus musculus cDNA, 3 end/clone = UI-M-AQ1-adx-c-06-0-UI/ | 1069.4 | P | 686.5 | P | 819.2 | P |
| | | clone_end = 3/gb = AI845584/gi = 5489490/ug = Mm 1791/len = 254/STRA = for | | | | | | |
| 2 | 93346_at | Cluster Incl M15668.Mus musculus X chromosome-linked phosphoglycerate kinase (pgk-1) mRNA, complete cds/ | 2966.7 | P | 2250.1 | P | 2480.9 | P |
| | | cds = (22,1275)/gb = M15668/gi = 202422/ug = Mm 188/len = 1653/STRA = for | | | | | | |
| 2 | 93528_s_at | Cluster Incl AI848050 UI-M-AH1-agp-g-10-0-UI.s1 Mus musculus cDNA, 3 end/clone = UI-M-AH1-agp-g-10-0-UI/ | 2011 | P | 745.3 | P | 1478.1 | P |
| | | clone_end = 3/gb = AI848050/gi = 5491956/ug = Mm 19788/len = 377/STRA = for | | | | | | |
| 2 | 93728_at | Cluster Incl X62940: Transforming growth factor beta 1 induced transcript 4/cds = (186,617)/gb = X62040/gi = 54928/ | 1849.7 | P | 1208.6 | P | 856.1 | P |
| | | ug = Mm.20927/len = 1706/STRA = for | | | | | | |
| 2 | 94048_at | Cluster Incl AW120683: UI-M-BH2.3-anz-c-06-0-UI.s1 Mus musculus cDNA, 3 end/clone = UI-M-BH2.3-anz-c-06-0-UI/ | 716.8 | P | 444.8 | P | 546.5 | P |
| | | clone_end = 3/gb = AW120683/gi = 6095981/ug = Mm 21981/len = 420/STRA = for | | | | | | |
| 2 | 94052_at | Cluster Incl AB013360 Mus musculus mRNA for DPM2, complete cds/cds = (24,278)/gb = AB013360/gi = 3790360/ | 1113.5 | P | 959.5 | P | 770.3 | P |
| | | ug = Mm 22001/len = 279/STRA = for | | | | | | |
| 2 | 94236_at | Cluster Incl AI838152 UI-M-AL0-abw-e-06-0-UI a1 Mus musculus cDNA, 3 end/clone = UI-M-AL0-abw-e-06-0-UI/ | 2240.4 | P | 1670.7 | P | 1568.3 | P |
| | | clone_end = 3/gb = AI838152/gi = 5472365/ug = Mm 22330/len = 451/STRA = for | | | | | | |
| 2 | 94255_g_at | Cluster Incl AI845237 UI-M-BG0-aht-g-09-0-UI s1 Mus musculus cDNA, 3 end/clone = UI-M-BG0-aht-g-09-0-UI/ | 1739.9 | P | 1526.3 | P | 1236.8 | P |
| | | clone_end = 3/gb = AI845237/gi = 5489143/ug = Mm 22409/len = 255/STRA = for | | | | | | |
| 2 | 94261_at | Cluster Incl AI836625. UI-M-AP0-abh-a-12-0-UI.s1 Mus musculus cDNA, 3 end/clone = UI-M-AP0-abh-a-12-0-UI/ | 2026.6 | P | 1699 | P | 1781.3 | P |
| | | clone_end = 3/gb = AI836625/gi = 5470838/ug = Mm 22440/len = 218/STRA = for | | | | | | |
| 2 | 94300_f_at | Cluster Incl AI839497: UI-M-AN0-acn-b-04-0-UI s1 Mus musculus cDNA, 3 end/clone = UI-M-AN0-acn-b-04-0-UI/ | 1147.1 | P | 1029.4 | P | 1092.4 | P |
| | | clone_end = 3/gb = AI839497/gi = 5473710/ug = Mm 22597/len = 385/STRA = for | | | | | | |
| 2 | 94309_g_at | Cluster Incl X70853: BM-90/fibulin extracellular matrix glyroprotein/cds = (0,353)/gb = X70853/gi = 396816/ug = Mm.22626/ | 2400.1 | P | 1207.3 | P | 1339.2 | P |
| | | len = 448/STRA = for | | | | | | |
| 2 | 95468_at | Cluster Incl AI850202 UI-M-BG1-aie-b-01-0-UI s1 Mus musculus cDNA, 3 end/clone = UI-M-BG1-aie-b-01-0-UI/ | 475. 4 | P | 443.3 | P | 304.8 | P |
| | | clone_end = 3/gb = AI850202/gi = 5494108/ug = Mm 24786/len = 401/STRA = for | | | | | | |
| 2 | 95471_at | Cluster Incl U22399 Cyclin-dependent kinase Inhibitor 1C (P57)/cds = (137,1144)/gb = U22399/gi = 790249/ug = Mm.2480/ | 5291.2 | P | 3100.6 | P | 4745.3 | P |
| | | len = 1462/STRA = for | | | | | | |
| 2 | 95474_at | Cluster Incl AW123850 UI-M-BH2 1-app-b-03-0-UI s1 Mus musculus cDNA, 3 end/clone = UI-M-BH2 1-app-b-03-0-UI/ | 959.6 | P | 349.6 | P | 510.2 | P |
| | | clone_end = 3/gb = AW123850/gi = 6099380/ug = Mm 24816/len = 347/STRA = for | | | | | | |
| 2 | 95482_at | Cluster Incl AW124247: UI-M-BH2 1-aph-f-08-0-UI s1 Mus musculus cDNA, 3 end/clone = UI-M-BH2 1-aph-f08-0-UI | 955 | P | 741.1 | P | 740.6 | P |
| | | clone_end = 3/gb = AW124247/gi = 6099777/ug = Mm 24870/len = 398/STRA = for | | | | | | |
| 2 | 96073_at | Cluster Incl U10435 Requiem/cds = (46,1161)/gb = U10435/gi = 606660/ug = Mm.2651/len = 2319/STRA = for | 550.3 | P | 424.7 | P | 508.3 | P |
| 2 | 96076_at | Cluster Incl AW121716: UI-M-BH2 3-aoc-h-05-0-UI s1 Mus musculus cDNA, 3 end/clone = UI-M-BH2.3-aoc-h-05-0-UI/ | 636.7 | P | 452.4 | P | 480.5 | P |
| | | clone_end = 3/gb = AW12176/gi = 6097049/ug = Mm 26558/len = 214/STRA = for | | | | | | |
| 2 | 96636_at | Cluster Incl AI852649.UI-M-BHO-aiu-h-10-0-UI s1 Mus musculus cDNA, 3 end/clone = UI-M-BH0-aiu-h-10-0-UI/ | 722.3 | P | 464.7 | P | 547.5 | P |
| | | clone_end = 3/gb = AI852649/gi = 5496555/ug = Mm 27270/len = 388/STRA = for | | | | | | |
| 2 | 97308_at | Cluster Incl AI835409 UI-M-AQ0-aaj-d-08-0-UI s1 Mus musculus cDNA, 3 end/clone = UI-M-AQ0-aaj-d-08-0-UI/ | 330.8 | P | 266.4 | P | 264.6 | P |
| | | clone_end = 3/gb = AI835409/gi = 5469622/ug = Mm 28063/len = 314/STRA = for | | | | | | |
| 2 | 97813_at | Cluster Incl M61909 Avian reticuloendotheliosis viral (v-rel) oncogene homolog A/cds = (60,1709)/gb = M61909/gi = 200025/ | 1830.2 | P | 1715.9 | P | 1525.5 | P |
| | | ug = Mm 28170/len = 2424/STRA = for | | | | | | |
| 2 | 97859_at | Cluster Incl AA762325 vw59d08 r1 M Mus musculus cDNA, 5 end/clone = IMAGE-1248111/clone_end = 5/gb = AA762325/ | 535.6 | P | 362 | P | 422 | P |
| | | gi = 2812072/ug = Mm 28308/len = 261/STRA = for | | | | | | |
| 2 | 98926_at | Cluster Incl AI838601 UI-M-AO0-abz-e-11-0-UI s1 Mus musculus cDNA, 3 end/clone = UI-M-AO0-abz-e-11-0-UI/ | 560.3 | P | 252 | P | 218 | P |
| | | clone_end = 3/gb = AI838601/gi = 5472814/ug = Mm 28643/len = 322/STRA = for | | | | | | |
| 2 | 98933_at | Cluster Incl AI849218 UI-M-AJ1-agz-b-02-0-UI s1 Mus musculus cDNA, 3 end/clone = UI-M-AJ1-agz-b-02-0-UI/ | 798.3 | P | 560.8 | P | 805.5 | P |
| | | clone_end = 3/gb = AI849218/gi = 5493124/ug = Mm 28678/len = 450/STRA = for | | | | | | |
| 2 | 94457_at | Cluster Incl AW049735 UI-M-BH1-ani-g-08-0-UI s1 Mus musculus cDNA, 3 end/clone = UI-M-BH1-ani-g-08-0-UI/ | 1082.4 | P | 705.9 | P | 650.6 | P |
| | | clone_end = 3/gb = AW049735/gi = 5910264/ug = Mm 28806/len = 510/STRA = for | | | | | | |
| 2 | 94482_at | Cluster Incl AF012251.Casein kinase II, alpha 2, polypeptide/cds = (98,1150)/gb = AF012251/gi = 2738574/ug = Mm 28881/ | 1889.9 | P | 1560.6 | P | 1528.6 | P |
| | | len = 1630/STRA = for | | | | | | |
| 2 | 94483_at | Cluster Incl AW060442 UI-M-BH1-anj-e-09-0-UI s1 Mus musculus cDNA, 3 end/clone = UI-M-BH1-anj-e-09-0-UI/ | 817.1 | P | 503.1 | P | 610.9 | P |
| | | clone_end = 3/gb = AW060442/gi = 6008193/ug = Mm 28881/len = 471/STRA = for | | | | | | |
| 2 | 94555_at | Cluster Incl AI845579.UI-M-AQ1-adx-b-12-0-UI s1 Mus musculus cDNA, 3 end/clone = UI-M-AQ1-adx-b-12-0-UI/ | 1532.5 | P | 915 | P | 980.3 | P |
| | | clone_end = 3/gb = AI845579/gi = 5489485/ug = Mm 29092/len = 254/STRA = for | | | | | | |
| 2 | 95044_at | Cluster Incl AI844549: UI-M-AJ1-ahf-h-10-0-UI s2 Mus musculus cDNA, 3 end/clone = UI-M-AJ1-ahf-h-10-0-UI/ | 824.6 | P | 557.4 | P | 780.8 | P |
| | | clone_end = 3/gb = AI844549/gi = 5488455/ug = Mm 29120/len = 430/STRA = for | | | | | | |
| 2 | 95057_at | Cluster Incl AI846938 UI-M-AK1-aey-e-10-0-UI s1 Mus musculus cDNA, 3 end/clone = UI-M-AK1-aey-e-10-0-UI/ | 610.2 | P | 592.6 | P | 484.1 | P |
| | | clone_end = 3/gb = AI846938/gi = 5490844/ug = Mm 29151/len = 342/STRA = for | | | | | | |
| 2 | 95081_at | Cluster Incl AI840176 UI-M-AO0-acc-f-04-0-UI s1 Mus musculus cDNA, 3 end/clone = UI-M-AO0-acc-f-04-0-UI/ | 2399.6 | P | 688.2 | P | 798.7 | P |
| | | clone_end = 3/gb = AI840176/gi = 5474389/ug = Mm 29253/len = 407/STRA = for | | | | | | |
| 2 | 95128_at | Cluster Incl AI849611.UI-M-AH1-agr-g-04-0-UI s1 Mus musculus cDNA, 3 end/clone = UI-M-AH1-agr-g-04-0-UI | 606.8 | P | 523.3 | P | 592.4 | P |
| | | clone_end = 3/gb = AI849611/gi = 5493517/ug = Mm 29412/len = 309/STRA = for | | | | | | |
| 2 | 95144_at | Cluster Incl AB024984.Mus musculus Sld329 mRNA, complete cds/cds = (134,1246)/gb = AB024984/gi = 5931548/ | 770.7 | P | 675.8 | P | 681.9 | P |
| | | ug = Mm 29453/len = 1595/STRA = for | | | | | | |
| 2 | 95161_at | Cluster Incl AW120628 UI-M-BH2 3-anz-a-04-0-UI.s1 Mus musculus cDNA, 3 end/clone = UI-M-BH2 3-anz-a-04-0-UI/ | 1230.8 | P | 883.1 | P | 1064.7 | P |
| | | clone_end = 3/gb = AW120628/gi = 6095961/ug = Mm.29490/len = 381/STRA = for | | | | | | |
| 2 | 95653_at | Cluster Incl AI841323 UI-M-AM0-adv-b-03-0-UI s1 Mus musculus cDNA, 3 end/clone = UI-M-AM0-adv-b-03-0-UI/ | 752.4 | P | 600.6 | P | 708.9 | P |
| | | clone_end = 3/gb = AI841323/gi = 5475538/ug = Mm.29517/len = 449/STRA = for | | | | | | |
| 2 | 95682_at | Cluster Incl AB026432 Mus musculus mRNA for XPE UV-damaged DNA binding factor, complete cds/cds = (81,3503)/ | 3857.4 | P | 3065.3 | P | 3507.1 | P |
| | | gb = AB026432/gi = 5931595/ug = Mm 29623/len = 4171/STRA = for | | | | | | |
| 2 | 95692_at | Cluster Incl AI852514 UI-M-BH0-aiw-c-06-0-UI s1 Mus musculus cDNA, 3 end/clone = UI-M-BH0-aiw-c-06-0-UI/ | 1049.7 | P | 913.9 | P | 796.5 | P |
| | | clone_end = 3/gb = AI852514/gi = 5496420/ug = Mm.29655/len = 298/STRA = for | | | | | | |
| 2 | 95731_at | Cluster Incl AI843106 UI-M-AK1-aes-e-01-0-UI s1 Mus musculus cDNA, 3 end/clone = .UI-M-AK1-aes-e-01-0-UI/ | 559.2 | P | 278 | P | 380.2 | P |
| | | clone_end = 3/gb = AI843106/gi = 5477319/ug = Mm.29742/len = 302/STRA = for | | | | | | |
| 2 | 96852_at | Cluster Incl AW122197.UI-M-BH2 2-aov-b-080-UI s1 Mus musculus cDNA, 3 end/clone = UI-M-BH2 2-aov-b-08-0-UI/ | 2155.2 | P | 1957.1 | P | 1735.9 | P |
| | | clone_end = 3/gb = AW122197/gi = 6097654/ug = Mm 30039/len = 436/STRA = for | | | | | | |
| 2 | 96876_at | Cluster Incl U34259 Mus musculus Golgi 4-transmembrane spanning transporter MTP mRNA, complete cds/ | 4113.9 | P | 3132.5 | P | 3293.7 | P |
| | | cds = (744,1445)/gb = U34259/gi = 1463119/ug = Mm 30071/len = 1984/STRA = for | | | | | | |
| 2 | 96926_at | Cluster Incl AA980164: ua31a05.r1 Mus musculus cDNA, 5 end/clone = IMAGE-1348304/clone_end = 5/gb = AA980164/ | 891.7 | P | 503.5 | P | 709.9 | P |
| | | gi = 3158700/ug = Mm.30162/len = 470/STRA = for | | | | | | |
| 2 | 97517_at | Cluster Incl AW061222: UI-M-BH1-amj-a-02-0-UI.s2 Mus musculus cDNA, 3 end/clone = UI-M-BH1-amj-a-02-0-UI | 1569.8 | P | 1171 | P | 958.2 | P |
| | | clone_end = 3/gb = AW061222/gi = 6008973/ug = Mm 3199/len = 143/STRA = for | | | | | | |
| 2 | 98060_at | Cluster Incl D49733 Lamin A/cds = (0,1997)/gb = D49733/gi = 1838920/ug = Mm.3438 len = 2916/STRA = for | 1670 | P | 1429.5 | P | 1565.2 | P |
| 2 | 98064_at | Cluster Incl AW125378 UI-M-BH2 3-aqh-b-04-0-UI s1 Mus musculus cDNA, 3 end/clone = UI-M-BH2 3-aqh-b-04-0-UI/ | 1031.2 | P | 648.4 | P | 861.3 | P |
| | | clone_end = 3/gb = AW125378/gi = 6100908/ug = Mm 34411/len = 447/STRA = for | | | | | | |
| 2 | 98067_at | Cluster Incl U09507: Cyclin-dependent kinase inhibitor 1A (P21)/cds = (200,679)/gb = U09507/gi = 595302/ug = Mm.34446/ | 1882.6 | P | 1466.1 | P | 1496.9 | P |
| | | len = 855/STRA = for | | | | | | |
| 2 | 98083_at | Cluster Incl AW049031 UI-M-BH1-amp-g-08-0-UI s1 Mus musculus cDNA, 3 end/clone = UI-M-BH1-amp-g-08-0-UI/ | 222.6 | P | 394.2 | P | 698 | P |
| | | clone_end = 3/gb = AW049031/gi = 5909560/ug = Mm 34576/len = 473/STRA = for | | | | | | |
| 2 | 98140_at | Cluster Incl X60961 Cadherin 1/cds = (127,2781)/gb = X60961/gi = 55133/ug = Mm 35605/len = 4413/STRA = for | 1433 | P | 1122 | P | 900.2 | P |
| 2 | 98579_at | Cluster Incl M28845 Early growth response 1/cds = (489,2090)/gb = M28845/gi = 1325981/ug = Mm 3765/len = 3476/ | 2708.2 | P | 982 | P | 835.6 | P |
| | | STRA = rev | | | | | | |
| 2 | 93605_at | Cluster Incl AI851163 UI-M-BH0-akg-f-05-0-UI.s1 Mus musculus cDNA, 3 end/clone = UI-M-BH0-akg-f-05-0-UI/ | 833.2 | P | 639.5 | P | 613.6 | P |
| | | clone_end = 3/gb = AI851163/gi = 5495034/ug = Mm.38433/len = 399/STRA = for | | | | | | |
| 2 | 99086_g_at | Cluster Incl AI021421 ub 12a08n r1 Mus musculus cDNA, 5 end/clone = IMAGE-1366742/clone_end = 5/gb = AI021421/ | 235.5 | P | 125.3 | P | 216.1 | P |
| | | gi = 3235757/ug = Mm 38976/len = 470/STRA = for | | | | | | |
| 2 | 99109_at | Cluster Incl M59821 Mouse growth factor-inducible protein (pip92) mRNA, complete cds/cds = (103,768)/gb = M59821/ | 3818.8 | P | 969.9 | P | 962 | P |
| | | gi = 200354/ug = Mm.399/len = 1515/STRA = for | | | | | | |
| 2 | 99183_at | Cluster Incl AW123013 UI-M-BH2 1-apb-e-03-0-UI s1 Mus musculus cDNA, 3 end/clone = UI-M-BH2 1-apb-e-03-0-UI/ | 777.4 | P | 505.2 | P | 532.2 | P |
| | | clone_end = 3/gb = AW123013/gi = 6098543/ug = Mm.41840/len = 553/STRA = for | | | | | | |
| 2 | 99566_at | Cluster Incl L31777 Triosephosphate isomerase/cds = (0,634)/gb = L31777/gi = 953213/ug = Mm 4222/len = 635/STRA = for | 5713.8 | P | 4357 | P | 4906.1 | P |
| 2 | 99574_at | Cluster Incl AI850887.UI-M-BH0-ajt-g-01-0-UI s1 Mus musculus cDNA, 3 end/clone = UI-M-BH0-ajt-g-01-0-UI/clone_end = 3/ | 587.7 | P | 451.9 | P | 388.5 | P |
| | | gb = AI850887/gi = 5494793/ug = Mm 423/len = 394/STRA = for | | | | | | |
| 2 | 99624_at | Cluster Incl AW125517.UI-M-BH2 2-aqm-f-07-0-UI s1 Mus musculus cDNA, 3 end/clone = UI-M-BH2 2-aqm-f-07-0-UI | 972.9 | P | 878.9 | P | 854.5 | P |
| | | clone_end = 3/gb = AW125517/gi = 6101047/ug = Mm 43309/len = 464/STRA = for | | | | | | |
| 2 | 99652_at | Cluster Incl AF109905 Mus musculus major histocompatibility locus class III regions Hsc701 gene, partial cds, smRNP, | 1344.8 | P | 1147.9 | P | 952.1 | P |
| | | G7A, NG23, MulS homolog, CLCP, NG24, NG25, and NG26 genes, complete cds, and unknown genes/cds = (0,1676)/ | | | | | | |
| | | gb = AF109905/gi = 3986751/ug = Mm 4/STRA = for | | | | | | |
| 2 | 99668_at | Cluster Incl U86405 Mus musculus brain amphiphysin 2 mRNA, complete cds/cds = (249,2015)/gb = U86405/gi = 1914840/ | 2546.4 | P | 1958.4 | P | 1912.1 | P |
| | | ug = Mm 4383/len = 2506/STRA = for | | | | | | |
| 2 | 100041_at | Cluster Incl AW124133: UI-M-BH2 1-apr-d-12-0-UI s1 Mus musculus cDNA, 3 end/clone = UI-M-BH2 1-apr-d-12-0-UI/ | 1457.4 | P | 1002.9 | P | 1089.2 | P |
| | | clone_end = 3/gb = AW124133/gi = 6099663/ug = Mm 44226/len = 133/STRA = for | | | | | | |
| 2 | 100056_at | Cluster Incl X54352: M domesticus MD6 mRNA/cds = (123,1391)/gb = X54352/gi = 49679/ug = Mm 4405/len = 1825/ | 366.6 | P | 289.2 | P | 282 | P |
| | | STRA = for | | | | | | |
| 2 | 100074_at | Cluster Incl AW046723: UI-M-BH1-alt-f-03-0-UI s1 Mus musculus cDNA, 3 end/clone = UI-M-BH1-alf-f-03-0-UI/ | 915.9 | P | 596.2 | P | 531.6 | P |
| | | clone_end = 3/gb = AW046723/gi = 5907292/ug = Mm 45233/len = 220/STRA = for | | | | | | |
| 2 | 100099_at | Cluster Incl Z14132 Sphingomyelin phosphodiesterase 1, acid lysosomal/cds = (0,1883)/gb = Z14132/gi = 475954/ | 1831.2 | P | 1573.2 | P | 1421.9 | P |
| | | ug = Mm 4628/len = 1884/STRA = for | | | | | | |
| 2 | 100133_at | Cluster Incl M27266 Fyn proto-oncogene/cds = (225,1829)/gb = M27266/gi = 193357/ug = Mm.4848/len = 2299/STRA = for | 601.6 | P | 245.3 | P | 300.1 | P |
| 2 | 100155_at | Cluster Incl L57509 Cell adhesion kinase/cds = (106,2841)/gb = L57509/gi = 1161062/ug = Mm.5021/len = 3674/STRA = for | 734.7 | P | 513.7 | P | 451.3 | P |
| 2 | 100561_at | Cluster Incl AW209098: un99g09 x1 Mus musculus cDNA, 3 end/clone = IMAGE-2582080/clone_end = 3/gb = AW209098/ | 1180.5 | P | 957.6 | P | 850.1 | P |
| | | gi = 6515038/ug = Mm 56685/len = 486/STRA = for | | | | | | |
| 2 | 100605_at | Cluster Incl M81086 Mouse skeletal muscle beta tropomyosin mRNA, complete cds/cds = (34,888)/gb = M81086/gi202162/ | 1435.3 | P | 974.9 | P | 1308.4 | P |
| | | ug = Mm 646/len = 2097/STRA = for | | | | | | |
| 2 | 100635_at | Cluster Incl L20294 Mus musculus GTP-binding protein (mSara) homologue mRNA, complete cds/cds = (84,680)/ | 2139 | P | 1610.5 | P | 1671.3 | P |
| | | gb = L20294/gi = 436563/ug = Mm 6698/len = 696/STRA = for | | | | | | |
| 2 | 101028_l_at | Cluster Incl M15501 Actin, alpha, cardiac/cds = (0,1127)/gb = M15501/gi = 191653/ug = Mm 686/len = 1226/STRA = for | 5937.2 | P | 1468.9 | P | 2204.6 | P |
| 2 | 101030_at | Cluster Incl X99963: M. musculus rhoB gene/cds = (0,590)/gb = X99963/gi = 1491740/ug = Mm 687/len = 591/STRA = for | 3346.8 | P | 1148.5 | P | 1370.9 | P |
| 2 | 101069_g_at | Cluster Incl AA656621: vr49a10 s1 Mus musculus cDNA, 5 end/clone = IMAGE-1123962/clone_end = 5/gb = AA656621/ | 2016.4 | P | 471.5 | P | 573.5 | P |
| | | gi = 2592775/ug = Mm 7198/len = 335/STRA = for | | | | | | |
| 2 | 101070_at | Cluster Incl AW125438 UI-M-BH2 3-aqh-g-08-0-UI s1 Mus musculus cDNA, 3 end/clone = UI-M-BH2 3-aqh-g-08-0-UI/ | 504.5 | P | 419.4 | P | 156.2 | P |
| | | clone_end = 3/gb = AW125438/gi = 6100968/ug = Mm 7198/len = 442/STRA = for | | | | | | |
| 2 | 101502_at | Cluster Incl X89749.M. musculus mRNA for mTGIF protein/cds = (279,1097)/gb = X89749/gi = 1495713/ug = Mm.8155 | 513.8 | P | 395.3 | P | 348.6 | P |
| | | len = 1186/STRA = for | | | | | | |
| 2 | 101554_at | Cluster Incl U57524 Mus musculus I kappa B alpha gene, exons 2-6, partial cds/cds = (0,717)/gb = U57524/gi = 4097334/ | 4243.9 | P | 563.2 | P | 1395.8 | P |
| | | ug = Mm 8884/len = 815/STRA = for | | | | | | |
| 2 | 101977_at | Cluster Incl AW121873 UI-M-BH2 3-aoi-d-02-0-UI.s1 Mus musculus cDNA, 3 end/clone = UI-M-BH2 3-aoi-d-02-0-UI/ | 1195.7 | P | 910.4 | P | 973.9 | P |
| | | clone_end = 3/gb = AW121873/gi = 6097247/ug = Mm 95709/len = 491/STRA = for | | | | | | |
| 2 | 102363_r_at | Cluster Incl U20735 Mus musculus transcription factor junB (junB) gene, 5 region and complete cds/cds = (294,1328)/ | 1678.5 | P | 510.6 | P | 694.6 | P |
| | | gb = U20735/gi = 965003/ug = Mm.1167/len = 1786/STRA = for | | | | | | |
| 2 | 102751_at | Cluster Incl M32745. Transforming growth factor, beta 3/cds = (610,1842)/gb = M32745/gi = 201949/ug = Mm 1291/len = 2879/ | 584 | P | 482 | P | 380.5 | P |
| | | STRA = for | | | | | | |
| 2 | 102792_at | Cluster Incl U55040 Mus musculus uracil-DNA glycosylase (ung) gene, exon 1, partial cds/cds = (27,311)/gb = U55040/ | 1212.9 | P | 912.8 | P | 929.8 | P |
| | | gi-1762315/ug = Mm 1393/len = 312/STRA = for | | | | | | |
| 2 | 102991_s_at | Cluster Incl AF100956 Mus musculus major histocompatibility locus class II region; Fas-binding protein Daxx (DAXX) gene, | 258.6 | P | 136.1 | P | 200.6 | P |
| | | partial cds; Bing1 (BING1), tapasin (tapasin), RalGDS-like factor (RLF), KE2 (KE2), BING4 (BING4), beta1,3-galactosyl | | | | | | |
| | | transferase (be/STRA = for | | | | | | |
| 2 | 102996_at | Cluster Incl U80227: Mus musculus ELL homolog mRNA, complete cds/cds = (5,1813)/gb = U80227/gi = 1947072/ | 536.8 | P | 367.4 | P | 507.3 | P |
| | | ug = Mm. 1552/len = 3023/STRA = for | | | | | | |
| 2 | 103031_g_at | Cluster Incl L31397: Dynamin/cds = (73,2658)/gb = L31397/gi = 487858/ug = Mm.16080/len = 3924/STRA = rev | 1817.4 | P | 432.6 | P | 860.4 | P |
| 2 | 103069_at | Cluster Incl AI853682 UI-M-BH0-ajq-h-03-0-UI s1 Mus musculus cDNA, 3 end/clone = UI-M-BH0-ajq-h-03-0-UI/ | 479.6 | P | 210.7 | P | 266 | P |
| | | clone_end = 3/gb = AI853682/gi = 5497628/ug = Mm 16813/len = 425/STRA = for | | | | | | |
| 2 | 103098_at | Cluster Incl AW045765 UI-M-BH1-akt-c-06-0-UI s1 Mus musculus cDNA, 3 end/clone = UI-M-BH1-akt-c-06-0-UI/ | 1404.5 | P | 898.8 | P | 821.5 | P |
| | | clone_end = 3/gb = AW045765/gi = 5906294/ug = Mm. 17533/len = 338/STRA = for | | | | | | |
| 2 | 103343_at | Cluster Incl AI845815 UI-M-AO1-aeg-b-02-0-UI S1 Mus musculus cDNA, 3 end/clone = UI-M-AO1-aeg-b-02-0-UI/ | 467.7 | P | 286.3 | P | 277.5 | P |
| | | clone_end = 3/gb = AI845815/gi = 5489721/ug = Mm 18228/len = 367/STRA = for | | | | | | |
| 2 | 103636_at | Cluster Incl AI840971 UI-M-AK0-adk-e-02-0-UI s1 Mus musculus cDNA, 3 end/clone = UI-M-AK0-adk-e-02-o-UI/ | 683.9 | P | 614.2 | P | 402.4 | P |
| | | clone_end = 3/gb = AI840971/gi = 5475184/ug = Mm 20324/len = 389/STRA = for | | | | | | |
| 2 | 104094_at | Cluster Incl Y08361 M musculus mRNA for RIL protein/cds = (36,1028)/gb = Y08361/gi = 1585268/ug = Mm.21830/len = 1135/ | 290.8 | P | 250.6 | P | 238.6 | P |
| | | STRA = for | | | | | | |
| 2 | 104116_at | Cluster Incl AW124049 UI-M-BH2 1-apn-e-03-0-UI s1 Mus musculus cDNA, 3 end/clone = UI-M-BH2 1-apn-e-03-0-UI/ | 596.8 | P | 230.8 | P | 306.6 | P |
| | | clone_end = 3/gb = AW124049/gi = 6099579/ug = Mm 21965/len = 382/STRA = for | | | | | | |
| 2 | 104149_at | Cluster Incl AI642048 ub75b05.x1 Mus musculus cDNA, 3 end/clone = IMAGE-1383537/clone_end = 3/gb = AI642048/ | 3353.7 | P | 739.9 | P | 1024.6 | P |
| | | gi = 4720523/ug = Mm 22188/len = 434/STRA = for | | | | | | |
| 2 | 104286_at | Cluster Incl AA795541.vq96e09 r1 Mus musculus cDNA, 5 end/clone = IMAGE-1110184/clone_end = 5/gb = AA795541/ | 985.6 | P | 451.4 | P | 601.3 | P |
| | | gi = 2858496/ug = Mm 22260/len = 393/STRA = for | | | | | | |
| 2 | 104302_f_at | Cluster Incl AI851085 UI-M-BH0-akg-b-04-0-UI.s1 Mus musculus cDNA,3 end/clone = UI-M-BH0-akg-b-04-0-UI/ | 656.1 | P | 503.8 | P | 583.5 | P |
| | | clone_end = 3/gb = AI851085/gi = 5494991/ug = Mm 22353/len = 486/STRA = for | | | | | | |
| 2 | 104352_at | Cluster Incl AI838366 UI-M-AI0-aak-f-10-0-UI.s2 Mus musculus cDNA, 3 end/clone = UI-M-AI0-aak-f-10-0-UI/clone_end = 3/ | 1127.2 | P | 1007.1 | P | 1139.6 | P |
| | | gb = AI838366/gi = 5472579/ug = Mm 22572/len = 319/STRA = for | | | | | | |
| 2 | 104533_at | Cluster Incl AA764261 W49f08.r1 Mus musculus cDNA, 5 end/clone = IMAGE-1225767/clone_end = 5/gb = AA764261/ | 1835.3 | P | 1297.1 | P | 1019.4 | P |
| | | gi = 2811783/ug = Mm 2322/len = 320/STRA = for | | | | | | |
| 2 | 104545_at | Cluster Incl U51866 Mus musculus casein kinase II alpha subunit mRNA, complete cds/cds = (203,1378)/gb = U51866/ | 802.5 | P | 278.1 | P | 439.1 | P |
| | | gi = 1262299/ug = Mm 23692/len = 1552/STRA = for | | | | | | |
| 2 | 104598_at | Cluster Incl X61940: Mouse mRNA for a growth factor-inducible immediate early gene (3CH134)/cds = (139,1242)/ | 3953.3 | P | 882.4 | P | 1130.5 | P |
| | | gb = X61940/gi = 49735/ug = Mm 2404/len = 1933/STRA = for | | | | | | |
| 2 | 104711_at | Cluster Incl AW122109 UI-M-BH2 2-aor-a-06-0-UI s1 Mus musculus cDNA, 3 end/clone = UI-M-BH2 2-aor-a-06-0-UI/ | 848.1 | P | 566.2 | P | 547.9 | P |
| | | clone_end = 3/gb = AW122109/gi = 6097524/ug = Mm 24437/len = 352/STRA = for | | | | | | |
| 2 | 94378_at | Cluster Incl U94828 Mus musculus retinally abundant regulator of G-protein signaling mRGS-r (RGS-r) mRNA, complete | 1026.2 | P | 499.7 | P | 373.9 | P |
| | | cds/cds = (109,714)/gb = U94828/gi = 2605641/ug = Mm 2555/len = 2250/STRA = for | | | | | | |
| 2 | 94957_at | Cluster Incl AW121154: UI-M-BH2 3-aoh-f-12-0-UI s1 Mus musculus cDNA, 3 end/clone = UI-M-BH2 3-aoh-f-12-0-UI/ | 275.4 | P | 89.3 | P | 224.4 | P |
| | | clone_end = 3/gb = AW121154/gi = 6096487/ug = Mm 27167/len = 421/STRA = for | | | | | | |
| 2 | 96128_at | Cluster Incl X69026: PCTAIRE-motif protein kinase 3/cds = (318,1673)/gb = X69026/gi = 53612/ug = Mm 28130/len = 2997/ | 452 | P | 421.8 | P | 379.9 | P |
| | | STRA = for | | | | | | |
| 2 | 96132_at | Cluster Incl AB023957 Mus musculus EIG 180 mRNA for ethanol induced gene product, complete cds/cds = (140,481)/ | 1896.2 | P | 1099.2 | P | 1003.2 | P |
| | | gb = AB023957/gi = 6468203/ug = Mm.28143/len = 728/STRA = for | | | | | | |
| 2 | 96147_at | Cluster Incl AW125070 UI-M-BH2 1-apx-d-08-0-UI s1 Mus musculus cDNA, 3 end/clone = UI-M-BH2 1-apx-d-08-0-UI/ | 390.7 | P | 295.9 | P | 274 | P |
| | | clone_end = 3/gb = AW125070/gi = 6100600/ug = Mm 28230/len = 438/STRA = for | | | | | | |
| 2 | 96723_f _at | Cluster Incl AW122911 UI-M-BH2-1-apa-f-09-0-UI s1 Mus musculus cDNA, 3 end/clone = UI-M-BH2 1-apa-f-09-0-UI/ | 805.8 | P | 692.2 | P | 593.2 | P |
| | | clone_end = 3/gb = AW122911/gi = 6098441/ug = Mm 28829/len = 366/STRA = for | | | | | | |
| 2 | 96742_at | Cluster Incl AA717826 vt98g09 r1 Mus musculus cDNA, 5 end/clone = IMAGE-1179232/clone_end = 5/gb = AA717826/ | 1034.8 | P | 635.4 | P | 767.6 | P |
| | | gi = 2730100/ug = Mm 28935/len = 255/STRA = for | | | | | | |
| 2 | 96797_s_at | Cluster Incl AW122681 UI-M-BH2 2-aot-a-02-0-UI s1 Mus musculus cDNA, 3 end/clone = UI-M-BH2 2-aot-a-02-0-UI/ | 1064.9 | P | 884.6 | P | 778.9 | P |
| | | clone_end = 3/gb = AW122681/gi = 6098211/ug = Mm 29170/len = 349/STRA = for | | | | | | |
| 2 | 96813_f_at | Cluster Incl AI852973 UI-M-BH0-aiz-g-09-0-UI s1 Mus musculus cDNA, 3 end/clone = UI-M-BH0-aiz-g-09-0-UI/ | 905.6 | P | 792.5 | P | 708.4 | P |
| | | clone_end = 3/gb = AI852973/gi = 5496879/ug = Mm 29297/len = 375/STRA = for | | | | | | |
| 2 | 97347_at | Cluster Incl AA838868 vw78c04.r1 Mus musculus cDNA, 5 end/clone = IMAGE-1261062/clone_end = 5/gb = AA838868/ | 696.2 | P | 410.8 | P | 447.1 | P |
| | | gi = 2914963/ug = Mm 29618/len = 493/STRA = rev | | | | | | |
| 2 | 97349_at | Cluster Incl AA727410 vu99g08 r1 Mus museulus cDNA, 5 end/clone = IMAGE-1210334/clone_end = 5/gb = AA727410/ | 172.2 | P | 903.3 | P | 552.8 | P |
| | | gi = 2745117/ug = Mm.2962/len = 506/STRA = for | | | | | | |
| 2 | 97366_at | Cluster Incl AI851024 UI-M-BH0-ajv-d-07-o-UI s1 Mus musculus cDNA, 3 end/clone = UI-M-BH0-ajv-d-07-0-UI/ | 709.6 | P | 384.5 | P | 578.1 | P |
| | | clone_end = 3/gb = AI851024/gi = 5494930/ug = Mm.29689/len = 132/STRA = for | | | | | | |
| 2 | 97370_at | Cluster Incl AI552922 vg73I08 y1 Mus musculus cDNA, 5 end/clone = IMAGE-871623/clone_end = 5/gb = AI552922/ | 446 | P | 287.1 | P | 376.9 | P |
| | | gi = 4485285/ug = Mm 29694/len = 222/STRA = fof | | | | | | |
| 2 | 97405_at | Cluster Incl M28489: Mus musculus (dona Mu6a) ribosomal pratotein S6 kinase (rsk) mRNA, complete cds/cds = (151,2325)/ | 801.4 | P | 614.3 | P | 727.3 | P |
| | | qb = M28489/gi = 556321/ug = Mm 29910/len = 3081/STRA = for | | | | | | |
| 2 | 97423_at | Cluster Incl AI839819 UI-M-AN0-acq-c-03-0-UI s1 Mus musculus cDNA, 3 end/clone = UI-M-AN0-acq-c-03-0-UI/ | 664 | P | 418.4 | P | 596.4 | P |
| | | clone_end = 3/gb = AI839819/gi = 5473997/ug = Mm.30006/len = 305/STRA = rev | | | | | | |
| 2 | 97429_at | Cluster Incl AW048113 UI-M-BH1-alq-d-12-0-UI s1 Mus musculus cDNA, 3 end/clone = UI-M-BH1-alq-d-12-0-UI/ | 574.2 | P | 395.1 | P | 423 | P |
| | | clone_end = 3/gb = W048113/gi = 5908642/ug = Mm.30079/len = 395/STRA = for | | | | | | |
| 2 | 97921_at | Cluster Incl AI846279 UI-M-AK1-aez-e-04-0-UI s1 Mus musculus cDNA, 3 end/clone = UI-M-AK1-aez-e-04-0-UI/ | 1200.2 | P | 885.7 | P | 929.3 | P |
| | | clone_end = 3/gb = AI846279/gi = 5490185/ug = Mm 30145/len = 409/STRA = for | | | | | | |
| 2 | 97943_at | Cluster Incl AI747133 ui12a02.x1 Mus musculus cDNA, 3 end/clone = IMAGE-2076554/clone_end = 3/gb = AI747133/ | 1384.2 | P | 858.3 | P | 745.9 | P |
| | | gi = 5125397/ug = Mm 30290/len = 560/STRA = for | | | | | | |
| 2 | 97982_at | Cluster Incl AA260139 va37a08 r1 Mus musculus cDNA, 5 end/clone = IMAGE-733526/clone_end = 5/gb = AA260139/ | 608.2 | P | 342.4 | P | 280 | P |
| | | gi = 1896624/ug = Mm 3127/len = 303/STRA = for | | | | | | |
| 2 | 98999_at | Cluster Incl AA606587.vm86b01 r1 Mus musculus cDNA, 5 end/clone = IMAGE-1005097/clone_end = 5/gb = AA606587/ | 395 | P | 274.7 | P | 158.8 | P |
| | | gi = 245521/ug = Mm 38151/len = 559/STRA = for | | | | | | |
| 2 | 99042_s_at | Cluster Incl U66918: Short stature homeobox 2/cds = (215,1210)/gb = U66918/gi = 1616757/ug = Mm.39093/len = 1603/ | 505 | P | 298.3 | P | 379.8 | P |
| | | STRA = for | | | | | | |
| 2 | 99052_at | Cluster Incl D76432: Transcriptrion factor 8/cds = (19,3372)/gb = D76432/gi = 1027499/ug = Mm 3929/len = 5214/STRA = rev | 415.5 | P | 337 | P | 300.5 | P |
| 2 | 99475_at | Cluster Incl UB8327: Mus musculus suppressor of cytokine signalling-2 (SOCS-2) mRNA, complete cds/cds = (222,818)/ | 600.9 | P | 330.8 | P | 457.5 | P |
| | | gb = U88327/gi = 2245385/ug = Mm.4132/len = 1121/STRA = for | | | | | | |
| 2 | 99978_s_at | Cluster Incl D83073 Mouse mRNA for p38b, complete cds/cds = (229,1311)/gb = D83073/gi = 1181606/ug = Mm 4437/ | 1282 | P | 1121.4 | P | 1022.6 | P |
| | | len = 3352/STRA = for | | | | | | |
| 2 | 100005_at | Cluster Incl X92346 Tnf receptor associated factor 4/cds = (130,1542)/gb = X92346/gi = 1041445/ug = Mm 4521/len:2043/ | 799.9 | P | 646.8 | P | 650.6 | P |
| | | STRA = for | | | | | | |
| 2 | 100017_at | Cluster Incl U68267: Mus musculus myosin binding protein H (MyBP-H) mRNA, complete cds/cds = (50,1501)/gb = U68267/ | 594.7 | P | 486.9 | P | 537.4 | P |
| | | gi = 1546812/ug = Mm 4562/len = 1850/STRA = for | | | | | | |
| 2 | 100022_at | Cluster Incl D89613: Cytokine inducible SH2-containing protein/cds = UNKNOWN/gb = D89613/gi = 2554633/ug = Mm 4592/ | 1285.3 | P | 892.9 | P | 986.1 | P |
| | | len = 1010/STRA = for | | | | | | |
| 2 | 100024_at | Cluster Incl AI641895vq44e08 y1 Mus musculus cDNA, 5 end/clone = IMAGE-1097126/clone_end = 5/gb = AI641895/ | 667 | P | 572 | P | 506.1 | P |
| | | gi = 4720370/ug = Mm 46014/len = 413/STRA = for | | | | | | |
| 2 | 100407_at | Cluster Incl L38580 Galanin/cds = (0,374)/gb = L38580/gi = 927115/ug = Mm.4655/len = 553/STRA = for | 651 | P | 454.8 | P | 443.6 | P |
| 2 | 100441_s_at | Cluster Incl X69064: Ankyrin 1, erythroid/cds = (0,731)/gb = X69064/gi = 311818/ug = Mm 4789/len = 1704/STRA = for | 222.5 | P | 66.8 | P | 122.9 | P |
| 2 | 100488_at | Cluster Incl M24119: Acid beta glucosidase/cds = (86,1633)/gb = M24119/gi = 193449/ug = Mm.5031/len = 1684/STRA = for | 995.3 | P | 957.1 | P | 713.4 | P |
| 2 | 100877_at | Cluster Incl AI194274 ul14c07 r1 Mus musculus cDNA, 5 end/clone = IMAGE-1851276/clone_end = 5/gb = AI194274/ | 1367.6 | P | 367.4 | P | 928.7 | P |
| | | gi = 3745481/ug = Mm 53/len = 480/STRA = for | | | | | | |
| 2 | 100899_s_at | Cluster Incl AI265210 ul97a10.x1 Mus musculus cDNA, 3 end/clone = IMAGE-1890330/clone_end = 3/gb = AI265210/ | 713.6 | P | 678.8 | P | 649.9 | P |
| | | gi = 3883368/ug = Mm 57157/len = 485/STRA = for | | | | | | |
| 2 | 100928_at | Cluster Incl X75285 Fibulin 2/cds = (50,3715)/gb = X75285/gi = 437046/ug = Mm 6120/len = 4377/STRA = for | 1671.2 | P | 756 | P | 1197.8 | P |
| 2 | 100960_g_at | Cluster Incl X99921 M musculus mRNA for S100 calcium-binding protein A13/cds = (60,356)/gb = X99921/gi = 1694840/ | 898.4 | P | 675.9 | P | 729.1 | P |
| | | ug = Mm 6523/len = 440/STRA = for | | | | | | |
| 2 | 101357_at | Cluster Incl X14971 Mouse mRNA for alpha-adaptin (A)/cds = (205,3138)/gb = X14971/gi = 49877/ug = Mm.6877/len = 3496/ | 628.4 | P | 504.8 | P | 456.7 | P |
| | | STRA = for | | | | | | |
| 2 | 102209_at | Cluster Incl AF087434.Mus musculus transcription factor NF-ATc isoform a (NF-ATca) mRNA, complete cds/cds = (0,2153/) | 269.2 | P | 110.5 | P | 91.1 | P |
| | | gb = AF087434/gi = 3643194/ug = Mm 10085/len = 3469/STRA = for | | | | | | |
| 2 | 102213_at | Cluster Incl AF051945 Mus musculus Xin mRNA, complete cds/cds = (140,5173)/gb = AF051945/gi = 2970645/ | 544.2 | P | 383.2 | P | 310.8 | P |
| | | ug = Mm 10117/len = 5800/STRA = for | | | | | | |
| 2 | 102216_at | Cluster Incl Y14334 Arachidonale 12-lipoxygenase, pseudogene 2/cds = (175,2280)/gb = Y14334/gi = 3043452/ | 703.4 | P | 559.5 | P | 504.1 | P |
| | | ug = Mm 10143/len = 2347/STRA = for | | | | | | |
| 2 | 102283_at | Clustar Incl U05245 T-cell lymphoma invasion and metastasis 1/cds = (506,5281)/gb = U05245/gi = 497638/ug = Mm.1211/ | 837.1 | P | 744.1 | P | 517.1 | P |
| | | 1en = 7292/STRA = for | | | | | | |
| 2 | 102683_at | Cluster Incl U76009 Zinc transporter 3/cds = (0,1025)/gb = U76009/gi = 1763379/ug = Mm.1396/len = 1026/STRA = for | 2112.8 | P | 1788.4 | P | 1492.4 | P |
| 2 | 102737_at | Cluster Incl U35233 Endothelin 1/cds = (30,638)/gb = U35233/gi = 1244501/ug = Mm 14543/len = 1770/STRA = for | 651.2 | P | 204.7 | P | 180 | P |
| 2 | 102955_at | Cluster Incl U83148 Mus musculus NFIL3/E4BP4 transaction factor mRNA, complete cds/cds = (0,1388)/gb = U83148/ | 301.5 | P | 153.1 | P | 177.3 | P |
| | | gi = 2076877/ug = Mm 1755/len = 1389/STRA = for | | | | | | |
| 2 | 103215_g_at | Cluster Incl AW048883 UI-M-BH1-amf-f-01-0-UI.s1 Mus musculus cDNA, 3 end/clone = UI-M-BH1-amf-l-01-0-UI/ | 1391.7 | P | 556.5 | P | 957.3 | P |
| | | clone_end = 3/gb = AW048883/gi = 5909412/ug = Mm 1980/len = 489/STRA = for | | | | | | |
| 2 | 103504_at | Cluster Incl AI837107.UI-M-AK0-adc-d-11-0-UI s1 Mus muaculus cDNA, 3 end/clone = UI-M-AK0-adc-d-11-0-UI/ | 433.8 | P | 362.9 | P | 409.6 | P |
| | | clone_end = 3/gb = AI637107/gi = 5471320/ug = Mm 22438/len = 460/STRA = for | | | | | | |
| 2 | 103736_at | Cluster Incl AI837786: UI-M-AK0-adl-e-02-0-UI s1 Mus musculus cDNA, 3 end/clone = UI-M-AK0-adl-e-02-0-UI/ | 523.7 | P | 337.3 | P | 377.9 | P |
| | | clone_end = 3/gb = AI837786/gi = 5471999/ug = Mm24385/len = 412/STRA = for | | | | | | |
| 2 | 104000_at | Cluster lncl AI181346.ub83d07.r1 Mus musculus cDNA, 5 end/clone = IMAGE-1395085/clone_end-5/gb = AM81346/ | 1719.6 | P | 1033.4 | P | 1499.1 | P |
| | | gi = 3731984/ug = Mm 26580/len = 474/STRA = for | | | | | | |
| 2 | 104155_f_at | Cluster Incl U19118 Mus musculus transcription factor LRG-21 mRNA, complete cds/cds = (167,712)/gb = U19118/ | 3525.8 | P | 289.8 | P | 198 | P |
| | | gi = 633751/ug = .Mm2706/len = 1897/STRA = for | | | | | | |
| 2 | 104435_at | Cluster Incl AI844072: vu70c06 x1 Mus musculua cDNA, 3 end/clone = IMAGE-1196746/clone_end = 3/gb = AI644072/ | 1336.3 | P | 1107.1 | P | 996 | P |
| | | gi = 4722547/ug = Mm 29257/len = 460/STRA = for | | | | | | |
| 2 | 104477_at | Cluster Incl AW047643: UI-M-BH1-alo-a-02-0-Ul.s1 Mus musculus cDNA. 3 end/clone = UI-M-BH1-alo-a-02-0-UI/ | 1109.7 | P | 131 | P | 142.3 | P |
| | | clone_end = 3/gb = AW047643/gl = 5908172/ug = Mm 29940/len = 375/STRA = for | | | | | | |
| 2 | 92185_at | Cluster Incl AI846023.UI-M-AP1-agl-g-02-0-Ul s1 Mus musculus cDNA, 3 end/clone = UI-M-AP1-agl-g-02-0-UI/ | 1057.5 | P | 429.2 | P | 487.5 | P |
| | | clone_end = 3/gb = AI846023/gi = 5489929/ug = Mm 3388/len = 482/STRA = for | | | | | | |
| 2 | 92216_at | Cluster Incl AF015260 Mus musculus Smad7 mRNA, complele cds/cds = (0,1280)/gb = AF015260/gi = 2460039/ | 302.4 | P | 134.6 | P | 203.2 | P |
| | | ug = Mm 34407/len = 1281/STRA = for | | | | | | |
| 2 | 92220_s_at | Cluster Incl U60884 Mus musculus SH3P9 mRNA, complele cds/cds = (178,1482)/gb = U60884/gi = 1438562/ug = Mm.34445/ | 946.7 | P | 570.8 | P | 772.7 | P |
| | | len = 1531/STRA = for | | | | | | |
| 2 | 92325_at | Cluster Incl AI326889 mj61doe.x1 Mus musculus cDNA, 3 end/clone = IMAGE-480587/clone_end = 3/gb = AI326889/ | 367.5 | P | 314.8 | P | 241.5 | P |
| | | gi = 4061318/ug = Mm 38494/len = 494/STRA = for | | | | | | |
| 2 | 92352_at | Cluster Incl AF108021.Mus musculus lysophospholipid receptor B3 (Ipb3) gene, complete cds/cds = (0,1136)/gb = AF108021/ | 625.8 | P | 605.3 | P | 483.7 | P |
| | | gi = 4324652/ug = Mm 39366/len.1137/STRA = for | | | | | | |
| 2 | 92376_at | Cluster Incl U71205 Mus musculus ril mRNA, complete cds/cds = (80,739)/gb = U71205/gi = 1656004/ug = Mm.4009/ | 958.8 | P | 828.7 | P | 856.8 | P |
| | | len = 1079/STRA = for | | | | | | |
| 2 | 92484_at | Cluster Incl Y15907 Mus musculus mRNA for myc-intron-binding proteln-1/cds = (84,7376)/gb = Y15907/gi = 2706548/ | 189 | P | 105.7 | P | 97.4 | P |
| | | ug = Mm 42157/len = 8095/STRA = rev | | | | | | |
| 2 | 92730_at | Cluster Incl L07284 Heparin binding epidermal growth factor-like growth factor/cds = (225,851)/gb = L07264/gl = 192999/ | 1162.2 | P | 279.3 | P | 370.8 | P |
| | | ug = Mm 4661/len1000/STR = for | | | | | | |
| 2 | 92968_at | Cluster Incl D87902: Mouse mRNA for ARF5, complete cds/cds = (49,591)/gb = D87902/gi = 1565214/ug = Mm.4996/len = 933/ | 1414.2 | P | 952.4 | P | 1095.6 | P |
| | | STRA = for | | | | | | |
| 2 | 93187_at | Cluster Incl AW048347: UI-M-BH1-akk-e-03-0-Ul.s1 Mus musculus cDNA, 3 end/clone = Ul-M-BH1-akk-e-03-0-Ul/ | 769.3 | P | 637.5 | P | 797.5 | P |
| | | clone end = 3/gb = AW048347/gl = 5908876/ug = Mm 5510/len = 458/STRA = for | | | | | | |
| 2 | 93197_at | Cluster Incl AF109905 Mus musculus major histocompatibility locus class III regions Hsc70t gene, partial cds; smRNP, | 2106.6 | P | 144.7 | P | 1571.5 | P |
| | | G7A, NG23, MutS homolog, CLCP, NG24, NG25, and NG26 genes, complete cds, and unknown genes/cds = (0,380)/ | | | | | | |
| | | gb = AF 109905/gi = 3986751/ug = Mm 56/STRA = for | | | | | | |
| 2 | 93434_at | Cluster Incl AF026074 Mus musculus sulfotransferase-related protein (SULT-X1) mRNA, complete cds/cds = (88,963)/ | 198.5 | P | 157.4 | P | 127.8 | P |
| | | gb = AF026074/gi = 2570901/ug = Mm 6562/len = 1173/STRA = for | | | | | | |
| 2 | 93852_at | Cluster Incl AW045443: UI-M-BH1-akr-f-12-0-UI s1 Mus musculus cDNA, 3 end/clone = UI-M-BM1-akr-f-12-0-UI/ | 757.1 | P | 536.1 | P | 446 | P |
| | | clone_end = 3/gb = AW045443/gi = 5906012/ug = Mm 87279/len = 292/STRA = for | | | | | | |
| 2 | 93922_g_at | Cluster Incl AI573601-ud46a12.y1 Mus musculus cDNA, 5 end/clone = IMAGE-1448926/clone_end = 5/gb = AI573601/ | 757.1 | P | 683.1 | P | 624.5 | P |
| | | gi = 4536975/ug = Mm 89855/len = 535/STRA = for | | | | | | |
| 2 | 93965_r_at | Cluster Incl AF038995 Mus musculus putative RNA helicase RCK mRNA, partial cds/cds = (0,1359)/gb = AF038995/ | 1067.5 | P | 594.4 | P | 819.3 | P |
| | | gi = 2745893/ug = Mm 97095/len = 1360/STRA = for | | | | | | |
| 2 | 95286_at | Cluster Incl D14077 Clusterin/cds = (91,1437)/gb = D14077/gi = 220601/ug = Mm 18700/len = 1663/STRA = for | 971.8 | P | 724 | P | 812.5 | P |
| 2 | 95345_at | Cluster Incl AJ012160 Mus musculus 5T4 oncofetal trophoblast glycoprotein gene/cds = (328,1608)/gb = AJ012160/ | 392.8 | P | 226 | P | 265 | P |
| | | gi = 3805948/ug = Mm.20864/len = 2329/STRA = for | | | | | | |
| 2 | 95383_at | Cluster Incl AI415418 mc4Be08 x1 Mus musculus cDNA, 3 end/clone = IMAGE-351782/clone_end = 3/gb = AI415418/ | 1562.6 | P | 853.3 | P | 933.2 | P |
| | | gi = 4258922/ug = Mm 23082/len = 455/STRA = rev | | | | | | |
| 2 | 95989_at | Cluster Incl AI448172 mo64107 x1 Mus musculus cDNA, 3 end/clone = IMAGE-558373/clone_end = 3/gb = AI448172/ | 1067.8 | P | 714.4 | P | 700.6 | P |
| | | gi = 4309126/ug = Mm 25049/len = 373/STRA = for | | | | | | |
| 2 | 97114_at | Cluster Incl U57999 Prosaposin/cds = (25,1698)/gb = U57999/gi = 1381581/ug = Mm 3363/len = 1961/STRA = for | 2211.2 | P | 1866.1 | P | 1443.4 | P |
| 2 | 97132_at | Cluster Incl AA189890 mu55b11 r1 Mus musculus cDNA,5 end/clone = IMAGE-643293/clone_end = 5/gb = AA189890/ | 1220.3 | P | 1172.8 | P | 948.8 | P |
| | | gi = 1776532/ug = Mm 34876/len = 344/STRA = rev | | | | | | |
| 2 | 97165_r_at | Cluster Incl C77278 C77278 Mus musculus cDNA, 3 end/clone = J0028D08/clone_end = 3/gb = C77278/gi = 2517608/ | 331.6 | P | 262.1 | P | 163.8 | P |
| | | ug = Mm 35523/len = 591/STRA = rev | | | | | | |
| 2 | 97193_at | Cluster Incl AA575098 vh82h08 r1 Mus musculus cDNA, 3 end/clone = IMAGE-893535/clone_end = 3/gb = AA575098/ | 303.2 | P | 154.2 | P | 159.9 | P |
| | | gi = 2349724/ug = Mm 36566/len = 336/STRA = rev | | | | | | |
| 2 | 99367_at | Cluster Incl AA434661 ve16g03 r1 Mus musculus cDNA, 5 end/clone = IMAGE-818356/clone_end = 5/gb = AA434661/ | 101.8 | P | 44.1 | P | 91 | P |
| | | gi = 2139575/ug = Mm 56829/len = 320/STRA = for | | | | | | |
| 2 | 99399_at | Cluster Incl U89486 Mus musculus agouti-related protein (Agrp) gene, complete cds/cds = (0,395)/gb = U89486/gi = 2351716/ | 521.9 | P | 449.5 | P | 432.9 | P |
| | | ug = Mm 56995/len = 396/STRA = for | | | | | | |
| 2 | 99851_at | Cluster Incl AA172988 ms58e02 r1 Mus musculus cDNA, 5 end/clone = IMAGE-615770/clone_end = 5/gb = AA172988/ | 1227.8 | P | 915.7 | P | 720 | P |
| | | gi = 1752041/ug = Mm 6436/len = 344/STRA = for | | | | | | |
| 2 | 95215_f_at | Cluster Incl D50527.Mouse mRNA for TI-225, complete cds/cds = (37,417)/gb = D50527/gi = 1167509/ug = Mm 331/len = 498/ | 3726.8 | P | 3385.6 | P | 325.9 | P |
| | | STRA = for | | | | | | |
| 2 | 92830_s_at | Cluster Incl X14678 Zinc finger protein 36/cds = (756,1307)/gb = X14678/gi = 54803/ug = Mm.1298/len = 2282/STRA = for | 2239.6 | P | 444.9 | P | 348.6 | P |
| 2 | 92838_at | Cluster Incl L33726 Fascin 1/cds = (78,1559)/gb = L33726/gi = 497774/ug = Mm.13194/len = 2634/STRA = for | 1656.6 | P | 1357.9 | P | 1417.7 | P |
| 2 | 92874_f_at | Cluster Incl AF071316 Mus musculus COP9 complex subunit 7a (COPS7a) mRNA, complete cds/cds = (158,985)/ | 677.7 | P | 363.8 | P | 329.4 | P |
| | | gb = AF071316/gi = 3309173/ug = Mm.1444/len = 1779/STRA = for | | | | | | |
| 2 | 94063_at | Cluster Incl X85991 M musculus mRNA for semaphorin B/cds = (65,2347)/gb = X85991/gi = 854325/ug = Mm.22061/ | 834.3 | P | 465 | P | 470.7 | P |
| | | len = 3046/STRA = for | | | | | | |
| 2 | 98910_at | Cluster Incl AI846118 UI-M-AK1-aew-e-07-0-UI.s1 Mus musculus cDNA, 3 end/clone = UI-M-AK1-aew-e-07-0-UI/ | 1909.1 | P | 1214.3 | P | 1328.5 | P |
| | | clone_end = 3/gb = AI846118/gi = 5489989/ug = Mm.28594/len = 234/STRA = for | | | | | | |
| 2 | 98916_at | Cluster Incl AI854289 UI-M-BH0-ajs-e-06-0-UI s1 Mus musculus cDNA, 3 end/clone = UI-M-BH0-ajs-e-06-0-UI/ | 289.3 | P | 278.5 | P | 204.3 | P |
| | | clone_end = 3/gb-AI854289/gi = 5498195/ug = Mm 28618/len = 490/STRA = for | | | | | | |
| 2 | 96848_at | Cluster Incl AW120756 UI-M-BH2 3-any-d-12-0-UI s1 Mus musculus cDNA, 3 end/clone = UI-M-BH2 3-any-d-12-0-UI/ | 152.7 | P | 110.9 | P | 90.3 | A |
| | | clone_end = 3/gb = AW120756/gi = 6096089/ug = Mm 30030/len = 407/STRA = for | | | | | | |
| 2 | 97489_at | Cluster Incl AI846739 UI-M-AO1-ael-b-05-0-UI s1 Mus musculus cDNA, 3 end/clone = UI-M-AO1-ael-b-05-0-UI/ | 511.1 | P | 379.1 | A | 339.3 | P |
| | | clone_end = 3/gb = AI846739/gi = 5490645/ug = Mm 30969/len = 279/STRA = for | | | | | | |
| 2 | 98561_at | Cluster Incl AJ242874 Mus musculus mRNA for slow skeletal muscle troponin 1 (Tnnl1 gene)/cds = (143,706)/gb = AJ242674/ | 1840.7 | P | 320.4 | P | 563.8 | P |
| | | gi = 5457404/ug = Mm 36900/len = 1054/STRA = for | | | | | | |
| 2 | 100086_at | Cluster Incl D00622 Low density lipoprotein receptor related protein, associated protein 1/cds = (0,1081)/gb = D00622/ | 387.7 | P | 130 | P | 113.9 | A |
| | | gi = 220433/ug = Mm 4560/len = 2902/STRA = for | | | | | | |
| 2 | 101583_at | Cluster Incl M64292B-cell translocation gene 2, anti-proliferative/cds = (86,542)/gb = M64292/gi = 202068/ug = Mm 903/ | 2159.7 | P | 1272 | P | 1360.8 | P |
| | | len = 2583/STRA = for | | | | | | |
| 2 | 103423_at | Cluster Incl AI846517 UI-M-AN1-aff-e-02-0-UI s1 Mus musculus cDNA, 3 end/clone = UI-M-AN1-aff-e-02-0-UI/ | 514.1 | P | 257.7 | P | 404.5 | P |
| | | clone_end = 3/gb = AI846517/gi-5490423/ug = Mm 18942/len = 349/STRA = for | | | | | | |
| 2 | 103622_at | Clusler Incl AW050255.UI-M-BH1-ans-b-11-0-UI s1 Mus musculus cDNA, 3 end/clone = UI-M-BH1-ans-b-11-0-UI/ | 525 | P | 353.5 | M | 405.8 | P |
| | | clone_end = 3/gb = AW050255/gi = 5910784/ug = Mm 20260/len = 397/STRA = for | | | | | | |
| 2 | 103906_f_at | Cluster Incl AI840868 UI-M-AM0-adm-a-01-0-UI s1 Mus musculus cDNA. 3 end/clone = UI-M-AM0-adm-a-01-0-UI/ | 237.2 | A | 199.6 | P | 182.6 | P |
| | | clone_end = 3/gb = AI840868/gi = 5475081/ug = Mm 21401/len = 346/STRA = for | | | | | | |
| 2 | 104701_at | Cluster Incl Y07836 M musculus mRNA for basic-helix-loop-helix protein/cds = (0,1214)/gb = Y07836/gi = 1707584/ | 690.5 | P | 424.2 | P | 296.3 | P |
| | | ug = Mm 2436/len = 1508/STRA = for | | | | | | |
| 2 | 94978_at | Cluster Incl AIS89775 UI-M-AN0-acq-f-06-0-UI s1 Mus musculus cDNA, 3 end/clone = UI-M-AN0-acq-f-06-0-UI/ | 1026.3 | P | 834.6 | P | 88.1 | P |
| | | clone_end = 3/gb = AI839775/gi = 5474028/ug = Mm 27269/len = 273/STRA = for | | | | | | |
| 2 | 96227_at | Cluster Incl X70533 Corticosteroid binding globulin/cds = (54,1247)/gb = X70533/gi = 298114/ug = Mm 2874/len = 1462/ | 942.9 | P | 555.9 | P | 860 | P |
| | | STRA = for | | | | | | |
| 2 | 96801_at | Cluster Incl AJ010108.Mus musculus mRNA for cytosolic adenylate Kinase/cds = (16,600)/gb = AJ010108/gi = 5725309/ | 466.1 | P | 119.5 | P | 279.4 | P |
| | | ug = Mm 29189/len = 1958/STRA = for | | | | | | |
| 2 | 97960_at | Cluster Incl AW125800: UI-M-BH2.2-aql-h-05-0-UI.s1 Mus musculus cDNA, 3 end/clone = UI-M-BH2.2-aql-h-05-0-UI/ | 389.3 | P | 170.9 | P | 155.3 | P |
| | | clone_end = 3/gb = AW125800/gi = 6101330/ug = Mm.30602/len = 326/STRA = for | | | | | | |
| 2 | 100952_at | Cluster Incl U47323: Stromal interaction molecule 1/cds = (193,2250)/gb = U47323/gi = 1493832/ug = Mm.645/len = 3192/ | 772 | P | 495.5 | P | 524.9 | P |
| | | STRA = for | | | | | | |
| 2 | 101408_at | Cluster Incl AF010499: Guanldinoacetate methyltransferase/cds = (110,820)/gb = AF010499/gi = 2555082/ug = Mm 7329/ | 483.6 | P | 261.5 | P | 180.6 | P |
| | | len = 939/STRA = for | | | | | | |
| 2 | 103731_at | Cluster Incl AI837319 UI-M-AK0-adf-c-02-0-UI.s1 Mus musculus cDNA, 3 end/clone = UI-M-AK0-adf-c-02-0-UI/ | 382.6 | P | 352.8 | P | 297.8 | P |
| | | clone end = 3/gb = AI837319/gi = 5471532/ug = Mm.24309/len = 418/STRA = rev | | | | | | |
| 2 | 104156_r_at | Cluster Incl U19118 Mus musculus transcription factor LRG-21 mRNA, complete cds/cds = (167,712)/gb = U19118/ | 826.8 | P | 357.2 | P | 211.8 | P |
| | | gi = 633751/ug = Mm 2706/len = 1897/STRA = for | | | | | | |
| 2 | 104262_at | Cluster Incl M60057: Parathyroid hormone-like peptide/cds = (25,552)/gb = M60057/gi = 200565/ug = Mm 28440/len = 1022/ | 789.2 | P | 627.8 | P | 254.6 | A |
| | | STRA = rev | | | | | | |
| 2 | 92202_g_at | Cluster Incl AI553024: vf37g06 y1 Mus musculus cDNA, 5 end/clone = IMAGE-846010/clone_end = 5/gb = AI553024/ | 790.2 | P | 310.9 | P | 539 | P |
| | | gi = 4485387/ug = Mm 34106/len = 367/STRA = for | | | | | | |
| 2 | 92235_g_at | Cluster Incl X66223: M musculus mRNA for retinoid X receptor-alpha (mRXR-alpha)/cds = (177,1580)/gb = X66223/ | 588.2 | P | 498.9 | P | 491.3 | P |
| | | gi = 54021/ug = Mm.3470/len = 2088/STRA = for | | | | | | |
| 2 | 92382_at | Cluster Incl U49739 Myosin VI/cds = (159,3956)/gb = U49739/gi = 1322279/ug = Mm 4040/len = 4602/STRA = for | 44.3 | P | −37.6 | P | −39.5 | P |
| 2 | 92956_at | Cluster Incl X74760 Notch gene homolog 3, (Drosophila)/cds = (59,7015)/gb = X74760/gi = 483580/ug = Mm 4945/len = 7943/ | 599.2 | A | 598.4 | P | 488.8 | P |
| | | STRA = for | | | | | | |
| 2 | 93921_at | Cluster Incl AI573601 ud46a12 y1 Mus musculus cDNA, 5 end/clone = IMAGE-1448926/clone_end = 5/gb = AI573601/ | 667.2 | P | 461.5 | P | 506 | P |
| | | gi = 4536975/ug = Mm.89855/len = 535/STRA = for | | | | | | |
| 2 | 93958_at | Cluster Incl AA086863 mm86g02 r1 Mus musculus cDNA, 5 end/clone = IMAGE-535346/clone_end = 5/gb = AA086863/ | 270.1 | P | 218 | P | 223.8 | P |
| | | gi = 1630072/ug = Mm.95627/len = 494/STRA = for | | | | | | |
| 2 | 94097_at | Cluster Incl AB025922 Mus musculus Gli1 mRNA, complete cds/cds = (171,3506)/gb = AB025922/gi = 6009643/ | 488.1 | P | 426.3 | P | 348.9 | P |
| | | ug = Mm 10081/len = 3686/STRA = for | | | | | | |
| 2 | 95889_at | Cluster Incl AI854770 UI-M-BH0-aka-c-09-0-UI s1 Mus musculus cDNA, 3 end/clone = UI-M-BH0-aka-c-09-0-UI/ | 604.3 | P | 394.2 | P | 349.9 | P |
| | | clone_end = 3/gb = AI854770/gi = 5498676/ug = Mm 24046/len = 417/STRA = for | | | | | | |
| 2 | 97762_f_at | Cluster Incl U12890 Killer cell lectin-like receptor, subfamily A, member 7/cds = (21,824)/gb = U12890/gi = 602409/ | 240.7 | P | 205 | P | 194 | P |
| | | ug = Mm 4131/len = 921/STRA = for | | | | | | |
| 2 | 98277_at | Cluster Incl AB017615: Mus musculus mRNA for Eos protein, complete cds/cds = (752,2353)/gb = AB017615/gi = 4062982/ | 692.6 | P | 559.6 | P | 505.3 | P |
| | | ug = Mm 42034/len = 2688/STRA = for | | | | | | |
| 2 | 100289_at | Cluster Incl U92885: Mus musculus LERK-3 (Epl3) gene, partial cds/cds = (0,564)/gb = U92885/gi = 2843098/ug = Mm 8547/ | 1476.1 | P | 913.6 | P | 884.8 | P |
| | | len = 859/STRA = for | | | | | | |
| 2 | 97652_at | Cluster Incl L07379 Growth hormone releasing hormone receptor/cds = UNKNOWN/gb = L07379/gi = 193510/ug = Mm 89928/ | 817.7 | A | 589.7 | P | 347 | P |
| | | len = 1320/STRA = for | | | | | | |
| 2 | 101712_at | Cluster Incl AJ009823: Mus musculus mRNA for P2X7 receptor subunit/cds = (11,1798)/gb = AJ009323/gi = 4007637/ | 595.2 | P | 508.8 | P | 545.6 | P |
| | | ug = Mm.42026/len = 1810/STRA = for | | | | | | |
| 2 | 102091_f_at | Cluster Incl AA407599.EST00874 Mus musculus cDNA, 3 end/clone = C0003E09/clone_end = 3/gb = AA407599/ | 134.5 | P | 120.1 | P | 99.2 | P |
| | | gi = 2065789/ug = Mm 42282/len = 285/STRA = rev | | | | | | |
| 2 | 93799_at | Cluster Incl AI845933 UI-M-AK1-aex-f-02-0-UI.s1 Mus musculus cDNA, 3 end/clone = UI-M-AK1-aex-f-02-0-UI/ | 1009.3 | P | 937.2 | P | 907.7 | P |
| | | clone_end = 3/gb = AI845933/gi = 5489839/ug = Mm 21407/len = 325/STRA = for | | | | | | |
| 2 | 94208_at | Cluster Incl AW045202 UI-M-BH1-alh-c-08-0-UI.s1 Mus musculus cDNA, 3 end/clone = UI-M-BH1-alh-c-08-0-UI/ | 354 | P | 54.8 | P | 105.3 | P |
| | | clone_end = 3/gb = AW045202/gi = 5905731/ug = Mm.2220/len = 430/STRA = for | | | | | | |
| 2 | 94274_at | Cluster Incl AI837415 UI-M-AP0-abm-f-06-0-UI s1 Mus musculus cDNA, 3 end/clone = UI-M-AP0-abm-f-06-0-UI/ | 719.4 | P | 477.3 | P | 197 | P |
| | | clone_end = 3/gb = AI837415/gi = 5471628/ug = Mm 22491/len = 360/STRA = for | | | | | | |
| 2 | 102362_l_at | Cluster Incl U20735: Mus musculus transcription factor junB (junB) gene, 5 region and complete cds/cds = (294,1328)/ | 2656.3 | P | 575 | P | 650.9 | P |
| | | gb = U20735/gi = 965003/ug = Mm 1167/len = 1786/STRA = for | | | | | | |
| 2 | 102371_at | Cluster Incl X16995 Hormone receptor/cds = (2,1807)/gb = X16995/gi = 53312/ug = Mm.119/len = 2351/STRA = for | 1864.1 | P | 295 | P | 189.5 | P |
| 2 | 104755_at | Cluster Incl AJ242778: Mus musculus mRNA for ABINI, (A20-binding inhibitor of NF-kappa B activation (large)/ | 612.5 | P | 535.1 | P | 535.4 | P |
| | | cds = (116,2059)/gb = AJ242778/gi = 4995752/ug = Mm 24782/len = 2812/STRA = rev | | | | | | |
| 2 | 94336_at | Cluster Incl AI838318 UI-M-AO0-aby-f-01-0-UI s1 Mus musculus cDNA, 3 end/clone = UI-M-AO0-aby-f-01-0-UI/ | 609.1 | P | 502.3 | A | 309.5 | P |
| | | clone_end = 3/gb = AI838318/gi = 5472531/ug = Mm 2498/len = 186/STRA = rev | | | | | | |
| 2 | 99991_at | Cluster Incl U31993.lnterleukin 17 receptor/cds = (120,2714)/gb = U31993/gi = 1161342/ug = Mm 4481/len = 3288/STRA = for | 224.4 | P | 195.4 | P | 186.4 | P |
| 2 | 103204_r_at | Cluster Incl AW259499 up28d02 x1 Mus musculus cDNA, 3 end/clone = IMAGE-2655651/clone_end = 3/gb = AW259499/ | 472 | P | 122.6 | M | 194 | P |
| | | gi = 6632417/ug = Mm.1911/len = 454/STRA = for | | | | | | |
| 2 | 103551_at | Cluster Incl AW124208 UI-M-BH2 1-aph-c-03-0-UI.s1 Mus musculus cDNA, 3 end/clone = UI-M-BH2.1-aph-c-03-0-UI/ | 251.7 | P | 65.9 | P | 167.6 | P |
| | | clone_end = 3/gb = AW124208/gi = 6099738/ug = Mm 23813/len = 384/STRA = for | | | | | | |
| 2 | 92500_at | Cluster Incl AB025412 Mus musculus mRNA for Ten-m3, complete cds/cds = (49,8196)/gb = AB025412/gi = 4760779/ | 69.3 | P | 0.1 | P | 24.7 | P |
| | | ug = Mm 42191/len = 8964/STRA = for | | | | | | |
| 2 | 92923_g_at | Cluster Incl U17291: Mus musculus transcription factor AP-2 (AP-2) gene, alternative/cds = (36,602)/gb = U17291/ | 536.1 | P | 431.7 | P | 114.8 | A |
| | | gi = 755199/ug = Mm 4845/len = 603/STRA = for | | | | | | |
| 2 | 93718_at | Cluster Incl Z22821 Mus musculus mRNA for Rab23 protein/cds = (20,733)/gb = Z22821/gi = 438161/ug = Mm 86744/ | 95.5 | P | 61.4 | A | 76.5 | P |
| | | len = 740/STRA = for | | | | | | |
| 2 | 93724_at | Cluster Incl AI596034 uk22a01.x1 Mus musculus cDNA, 3 end/clone = IMAGE-1969704/clone_end = 3/gb = AI596034/ | 214.1 | P | 181.1 | A | 166.4 | P |
| | | gi = 4605082/ug = Mm.86922/len = 516/STRA = rev | | | | | | |
| 2 | 94133_r _at | Cluster Incl U94423: Mus musculus MEF2A mRNA, partial 3 UTR sequence/cds = UNKNOWN/gb = U94423/gi = 2088559/ | 97 | P | 82.6 | P | 40.6 | P |
| | | ug = Mm 11087/len = 1016/STRA = rev | | | | | | |
| 2 | 95360_at | Cluster Incl AA259683: va36d09 r1 Mus musculus cDNA, 5 end/clone = IMAGE-733457/clone_end = 5/gb = AA259683/ | 60 | P | −2.3 | P | 19.5 | P |
| | | gi = 1896186/ug = Mm.21902/len = 349/STRA = for | | | | | | |
| 2 | 97171_f_at | Cluster Incl AI060627 Ub42b11.r1 Mus musculus cDNA, 5 end/clone = IMAGE-1380381/clone_end = 5/gb = AI060627/ | 697.7 | P | 95.7 | A | 277.4 | P |
| | | gi = 3336050/ug = Mm 35621/len = 335/STRA = for | | | | | | |
| 2 | 98291_at | Cluster Incl AF092050: Mus musculus beta-1,3-N-acetylglucosaminyltransferase mRNA, complete cds/cds = (4,981)/ | 402.9 | P | 242.5 | P | 212.3 | P |
| | | gb = AF092050/gi = 4191391/ug = Mm.42092/len = 1536/STRA = for | | | | | | |
| 2 | 101769_at | Cluster Incl AB008178: Mus musculus CNR3 mRNA for Cadherin-related neural recepter 3, partial cds/cds = (0,2730)/ | 1357.2 | P | 1091.4 | P | 1016.6 | P |
| | | gb = AB008178/gi = 3253078/ug = Mm.57101/len = 2731/STRA = for | | | | | | |
| 2 | 101603_g_at | Cluster Incl C79108: C79108 Mus musculus cDNA, 3 end/clone = J0060E04/clone_end = 3/gb = C79108/gi = 2519438/ | 305.9 | P | 208.5 | P | 294 | P |
| | | ug = Mm 5769/len = 579/STRA = rev | | | | | | |
| 2 | 92768_s_at | Cluster Incl M15268.Aminolevulinic acid synthase 2, erythroid/cds = (0,1760)/gb = M15268/gi = 191857/ug = Mm 1217/ | 2789.4 | P | 139.3 | A | 356.3 | P |
| | | len = 1896/STRA = for | | | | | | |
| 2 | 93073_at | Cluster Incl U36576 Nuclear factor of activated T-cells, cytoplasmic 2/cds = (70,2853)/gb = U36576/gi = 1353400 | 1461.5 | P | 862.8 | P | 870.8 | P |
| | | ug = Mm 1574/len = 3518/STRA = rev | | | | | | |
| 2 | 93583_s_at | Cluster Incl V00817: Mouse germ line gene fragment for mu-immunoglobulin C-terminus (secreted form)/cds = (0,83)/ | 635 | P | 408.8 | M | 560.4 | P |
| | | gb = V00817/gi = 52345/ug = Mm 2066/len = 210/STRA = for | | | | | | |
| 2 | 94046_at | Cluster Incl U05247 C-src tyrosine kinase/cds = (0,1352)/gb = U05247/gi = 452471/ug = Mm.21974/len = 1779/STRA = for | 678.5 | P | 562.9 | A | 613.1 | P |
| 2 | 94856_r_at | Cluster Incl AI842362 UI-M-AM1-agc-a-03-0-UI s1 Mus musculus cDNA, 3 end/clone = UI-M-AM1-agc-a-03-0-UI/ | 142.5 | P | 94.7 | P | 69.1 | A |
| | | clone_end = 3/gb = AI842362/gi = 5476575/ug = Mm 23566/len = 298/STRA = for | | | | | | |
| 2 | 96026_at | Cluster Incl AA987153: uc80h10.x1 Mus musculus cDNA, 3 end/clone = IMAGE-1432003/clone_end = 3/gb = AA987153/ | 203.6 | P | 135.8 | P | 200.8 | P |
| | | gi = 3167916/ug = Mm.2573/len = 824/STRA = for | | | | | | |
| 2 | 97906_at | Cluster Incl Z19581.Seven in absentia 2/cds = (289,1266)/gb = Z19581/gi = 297036/ug = Mm 2847/len = 2198/STRA = for | 633.2 | P | 440.5 | P | 527.6 | P |
| 2 | 99186_at | Cluster Incl X75483 Cyclin A2/cds = (121,1389)/gb = X75483/gi = 414061/ug = Mm.4189/len = 1662/STRA = for | 754.8 | P | 707.2 | A | 515 | P |
| 2 | 101063_at | Cluster Incl M29793: M musculus slow/cardiac troponin C (cTnC) mRNA, complete cds/cds = (43,528)/gb = M29793/ | 842.7 | P | 113.4 | A | 350.9 | P |
| | | gi = 192839/ug = Mm 712/len = 703/STRA = for | | | | | | |
| 2 | 101505_at | Cluster Incl AW121234 UI-M-BH2 3-aok-f-04-0-UI s1 Mus musculus cDNA, 3 end/clone = UI-M-BH2 3-aok-f-04-0-UI/ | 229.9 | A | 101.8 | P | 198.3 | A |
| | | clone_end = 3/gb = AW121234/gi = 6096567/ug = Mm 8195/len = 426/STRA = for | | | | | | |
| 2 | 94947_g_at | Cluster Incl U43187: Mus musculus MEK kinase 3, mRNA, partial cds/cds = (331,2211)/gb = U43187/gi = 1223901/ | 140.4 | P | 108.6 | P | 113 | P |
| | | ug = Mm 27041/len = 3332/STRA = for | | | | | | |
| 2 | 97358_at | Cluster Incl AI851356 UI-M-BH0-akh-d-11-0-UI.s1 Mus musculus cDNA, 3 end/clone = UI-M-BH0-akh-d-11-0-UI/ | 566.4 | P | 365 | A | 429.1 | P |
| | | clone_end = 3/gb = AI851356/gi = 5495262/ug = Mm.29638/len = 401/STRA = for | | | | | | |
| 2 | 97942_g_at | Cluster Incl Y12582 M. musculus mRNA for calpain-like protease/cds = (129,2054)/gb = Y12582/gi = 2239139/ug=.Mm 30290/ | 762.2 | P | 121.5 | A | 311.8 | P |
| | | len = 2069/STRA = for | | | | | | |
| 2 | 100432_f_at | Cluster Incl U57820 MyoD family inhibitor/cds = (30,770)/gb = U57820/gi = 1545983/ug = Mm 4763/len = 1360/STRA = for | 1361 | P | 972.6 | A | 1138.4 | P |
| 2 | 93669_f_at | Cluster lncl AW107922 um24d07 x1 Mus musculus cDNA, 3 end/clone = IMAGE-2225485/clone_end = 3/gb = AW107922/ | 130.9 | P | 33.9 | P | 38.1 | P |
| | | gi = 6078722/ug = Mm 80595/len = 576/STRA = rev | | | | | | |
| 2 | 94770_at | Cluster Incl AI553596 vw47f05.x1 Mus musculus cDNA, 3 end/clone = IMAGE-1246977/clone_end = 3/gb = AI553596/ | 301.5 | P | 286.1 | A | 224.2 | P |
| | | gi = 4485959/ug = Mm 1648/len = 341/STRA = for | | | | | | |
| 2 | 95340_at | Cluster Incl M93310 Mouse metailothionein-III gene, complete cds/cds = (56,262)/gb = M93310/gi = 199133/ug = Mm 2064/ | 1549.9 | P | 870 | M | 989.8 | P |
| | | len = 401/STRA = for | | | | | | |
| 2 | 97769_at | Cluster Incl D17433 Prostaglandin F receptor/cds = (223,1323)/gb = D17433/gi = 457656/ug = Mm.4170/len = 2170/ | 264.8 | P | 233.5 | P | 229.5 | P |
| | | STRA = rev | | | | | | |
| 2 | 100736_at | Cluster Incl L77900 Mus musculus Barx2 mRNA, complete cds/cds = (67,753)/gb = L77900/gi = 2055363/ug = Mm 3508/ | 134.5 | A | 54 | P | 90.6 | P |
| | | len = 1274/STRA = for | | | | | | |
| 2 | 92619_at | Cluster Incl U40825 Mus musculus WW-domain binding protein 1 mRNA, complete cds/cds = (152,1069)/gb = U40825/ | 1592.1 | P | 1145.1 | P | 1293.7 | M |
| | | gi = 1777576/ug = Mm 1109/len = 1292/STRA = for | | | | | | |
| 2 | 92820_at | Cluster Incl AI846522 UI-M-AN1-aff-e-09-0-UI.s1 Mus musculus cDNA, 3 end/clone = UI-M-AN1-aff-e-09-0-UI/ | 700.7 | P | 369.9 | A | 624.8 | P |
| | | clone_end = 3/gb = AI846522/gi = 5490428/ug = Mm 12914/len = 370/STRA = rev | | | | | | |
| 2 | 93507_at | Cluster Incl X62622 Tissue inhibitor of metalloproteinase 2/cds = (226,888)/gb = X62622/gi = 54801/ug = Mm 19191/ | 321.8 | P | 230.8 | M | 262.1 | P |
| | | len = 1714/STRA = for | | | | | | |
| 2 | 94815_at | Cluster Incl X13586 Murine mRNA for 2,3-bisphosphoglycerate mutase (BPGM; EC 5.4,2.4)/cds = (20,799)/gb = X13586/ | 456.5 | P | 85.8 | A | 83.7 | A |
| | | gi = 50183/ug = Mm 22706/len = 825/STRA = for | | | | | | |
| 2 | 94906_at | Cluster Incl M22679 Mouse alcohol dehydrogenase class I gene (ADH-A-2)/cds = (0,1127)/gb = M22679/gi = 191715/ | 1280.4 | P | 222.8 | A | 224.7 | A |
| | | ug = Mm.2409/len = 1128/STRA = for | | | | | | |
| 2 | 101046_at | Cluster Incl X56397: Mouse mRNA for vimentin/cds = (84,1484)/gb = X56397/gi = 55407/ug = Mm 7/len = 1748/STRA = for | 562.8 | P | 367.3 | A | 248.2 | A |
| 2 | 101531_at | Cluster Incl AI527354.uj51b11.x1 Mus musculus cDNA, 3 end/clone = IMAGE-1923453/clone_end = 3/gb = AI527354/ | 1976.7 | P | 265.5 | A | 625 | P |
| | | gi = 4441489/ug = Mm 87581/len = 534/STRA = rev | | | | | | |
| 2 | 102429_at | Cluster Incl AB005451: Mus musculus mRNA for RST, complete cds/cds = (99,1760)/gb = AB005451/gi = 2696708/ | 906.7 | P | 483.3 | M | 581.4 | P |
| | | ug = Mm.12846/len = 1779/STRA = for | | | | | | |
| 2 | 104047_at | Cluster Incl AW122136: UI-M-BH2.2-aor-d-08-0-UI s1 Mus musculus cDNA, 3 end/clone = UI-M-BH2 2-aor-d-08-0-UI/ | 331.1 | M | 302 | P | 198.2 | A |
| | | clone end = 3/gb = AW122136/gi = 6097563/ug = Mm 21495/len = 276/STRA = for | | | | | | |
| 2 | 99021_at | Cluster Incl U03873 Paired masoderm homeobox 1/cds = (1021,1674)/gb = U03873/gi = 460124/ug = Mm 3869/len = 3359/ | 125.6 | P | 87.6 | P | 49.9 | P |
| | | STRA = for | | | | | | |
| 2 | 99055_at | Cluster Incl X94000: M. musculus Gal beta-1,3-GaINAc-specific GaINAc alpha-2,6-aiatyltransferase gene (1804 bp)/ | 1489.3 | A | 691.9 | A | 610.8 | M |
| | | cds = (67,153)/gb-X94000/gi = 1526610/ug = Mm.3947/len = 154/STRA = for | | | | | | |
| 2 | 100449_g_at | Cluster Incl Z31664 M musculus (NIH Swiss) mRNA for ALK-1/cds = (240,1748)/gb = Z31684/gi = 840814/ug = Mm 4839/ | 630.6 | P | 418.3 | A | 388.6 | P |
| | | len = 1909/STRA = for | | | | | | |
| 2 | 103211_at | Cluster Incl AI852574: UI-M-BH0-aiu-a-02-0-UI s1 Mus musculus cDNA, 3 end/clone = UI-M-BH0-alu-a-02-0-UI/ | 1155.5 | P | 876.5 | P | 631.4 | A |
| | | clone_end = 3/gb = AI852574/gi = 5496480/ug = Mm 1960/len = 235/STRA = rev | | | | | | |
| 2 | 103789_at | Cluster Incl AI854614: UI-M-BH0-ake-d-06-0-UI s1 Mus musculus cDNA, 3 end/clone = UI-M-BH0-ake-d-06-0-UI/ | 562.1 | A | 339.9 | P | 196 | P |
| | | clone_end = 3/gb = AI854614/gi = 5498520/ug = Mm 24948/len = 446/STRA = for | | | | | | |
| 2 | 92532_at | Cluster Incl D49730: Mouse mRNA for V1a agrinine vasopression receptor, complete cds/cds = (312,1583)/gb = D49730/ | 440.9 | P | 321.7 | P | 305.8 | A |
| | | gi = 1722700/ug = Mm.4351/len = 2684/STRA = rev | | | | | | |
| 2 | 93870_at | Cluster Incl M64429 Braf transforming gene/cds = (37,2016)/gb = M64429/gi = 553877/ug = Mm 8807/len = 2050/STRA = for | 138.4 | P | 103.6 | P | 98.7 | A |
| 2 | 94142_at | Cluster Incl M13926 Colony stimulating factor, granulocyte/cds = (87,693)/gb = M13926/gi = 193451/ug = Mm 1238/len = 1363/ | 1000.2 | P | 580.1 | P | 459.4 | A |
| | | STRA = for | | | | | | |
| 2 | 94784_at | Cluster Incl AI593484 vu42d01 x1 Mus musculus cDNA, 3 end/clone = IMAGE-1194049/clone_end = 3/gb = AI593484/ | 321.1 | P | 259.7 | P | 166.9 | M |
| | | gi = 4602532/ug = Mm.1689/len = 477/STRA = for | | | | | | |
| 2 | 95881_f_at | Cluster Incl AI447783 mq84c02.x1 Mus musculus cDNA, 3 end/clone = IMAGE-585410/clone_end = 3/gb = AI447783/ | 462.7 | M | 396.1 | P | 300 | P |
| | | gi = 4306405/ug = Mm.23936/len = 347/STRA = rev | | | | | | |
| 2 | 96574_at | Cluster Incl M30136 Interleukin 9/cds = (0,434)/gb = M30136/gi = 200190/ug = Mm 3006/len = 435/STRA = for | 391.6 | P | 318.8 | A | 259.1 | P |
| 2 | 97147_at | Cluster Incl C80892: C80892 Mus musculus cDNA, 3 end/clone = J0090C03/clone_end = 3/gb = C80892/gi = 2521222/ | 66.9 | P | 34.3 | M | 75.8 | P |
| | | ug = Mm 34947/len = 585/STRA = rev | | | | | | |
| 2 | 100291_at | Cluster Incl X57111.Casitas B-lineage lymphoma/cds = (96,2786)/gb = X57111/gi = 50314/ug = Mm.8585/len = 2808/ | 252.4 | P | 102.9 | P | 89.9 | P |
| | | STRA = for | | | | | | |
| 2 | 94270_at | Cluster Incl M22832 Mus musculus cytokeratin (endoB) gene, complete cds/cds = (58,1329)/gb = M22832/gi = 340757/ | 202.6 | A | 144.3 | P | 211.2 | P |
| | | ug = Mm 22479/len = 1373/STRA = for | | | | | | |
| 2 | 94308_at | Cluster Incl X70853.BM-90/fibulin extracellular matrix glyroprotein/cds = (0,353)/gb = X70853/gi = 396816/ug = Mm 22626/ | 665.6 | P | 480.8 | M | 592.5 | P |
| | | len = 448/STRA = for | | | | | | |
| 2 | 100130_at | Cluster Incl X12761: Jun oncogene/cds = (354,1358)/gb = X12761/gi = 52758/ug = Mm 482/len = 2565/STRA = for | 1006.3 | P | 135.9 | A | 187.6 | A |
| 2 | 102071_at | Cluster Incl AIB51565: UI-M-BHO-aki-f-01-0-UI s1 Mus musculus cDNA, 3 end/clone = UI-M-BH0-aki-f-01-0-UI/clone_end = 3/ | 659.3 | P | 481.9 | M | 528.2 | P |
| | | gb = AI851565/gi = 5495471/ug = Mm 10641/len = 341/STRA = for | | | | | | |
| 2 | 103088_at | Cluster Incl X94310: M musculus mRNA for L1-like protein/cds = (295,3924)/gb = X94310/gi = 1532034/ug = Mm 1736/ | 144.8 | P | 108.2 | P | 59.8 | A |
| | | len = 4437/STRA = for | | | | | | |
| 2 | 103420_at | Cluster Incl U79753 Emerin/cds = (106,885)/gb = U79753/gi = 1916802/ug = Mm 18892/len = 1270/STRA = for | 1000.4 | P | 860.2 | P | 870.5 | A |
| 2 | 104393_at | Cluster Incl AI843571 UI-M-AO1-aen-d-09-0-UI.s1 Mus musculus cDNA, 3 end/clone = UI-M-AO1-aen-d-09-0-UI/ | 105.1 | P | −98.8 | M | −64.2 | A |
| | | clone_end = 3/gb = AI843571/gl = 5477824/ug = Mm 2275/len = 290/STRA = for | | | | | | |
| 2 | 95566_at | Cluster Incl AF050666: Glycosylphosphatidytinositol specific phospholipase D1/cds = (270,2783)/gb = AF050666/ | 554.4 | P | 305 | A | 313 | A |
| | | gi = 2984690/ug = Mm.2779/len = 3436/STHA = for | | | | | | |
| 2 | 100015_at | Cluster Incl X67677 Yamaguchi sarcoma viral (v-yes) oncogene homolog/cds = (602,2227)/gb = X67677/gi = 50623/ | 428.2 | A | 242 | A | 426.4 | A |
| | | ug = Mm 4558/len = 4550/STRA = for | | | | | | |
| 2 | 102202_s_at | Cluster Incl M36411 Mpv17 transgene, kidney disease mutant/cds = (48,578)/gb = M36411/gi = 199789/ug = Mm 999/ | 226.7 | P | 191.8 | A | 167.9 | A |
| | | len = 1411/STRA = rev | | | | | | |
| 2 | 103214_at | Cluster Incl AW048883 UI-M-BH1-amf-f-01-0-UI s1 Mus musculus cDNA, 3 end/clone = UI-M-BH1-amf-f-01-0-UI/ | 219.9 | P | 5.1 | A | 134.8 | P |
| | | clone_end = 3/gb = AW048883/gi = 5909412/ug = Mm 1980/len = 489/STRA = for | | | | | | |
| 2 | 104455_at | Cluster Incl J03057 Mouse brain Ca++/calmodulin-dependant protein kinase type IV (CAM kinase IV) mRNA, 3 end/ | 148.5 | P | 84.5 | M | 75.7 | P |
| | | cds = (0,667)/gb = J03057/gi = 192366/ug = Mm 2951/len = 773/STRA = for | | | | | | |
| 2 | 93619_at | Cluster Incl AF022992 Period homolog (Drosophila)/cds = (225,4100)/gb = AF022992/gi = 2435508/ug = Mm 7373/len = 4701/ | 454.7 | A | 168 | A | 345.8 | P |
| | | STRA = for | | | | | | |
| 2 | 94748_g_at | Cluster Incl M34397.Interieukin 3 receptor, beta chain 1/cds = (287,2977)/gb = M34397/gi = 191821/ug = Mm 14595/ | 343 | A | 250.1 | P | 200.9 | P |
| | | len = 3113/STRA = for | | | | | | |
| 2 | 96592_at | Cluster Incl U50413 Phosphatidylinositol 3-kinase, regulatory subunit, polypeptide 1 (p85 alpha)/cds = (574,2748)/ | 159.4 | A | 131.6 | P | 79.1 | P |
| | | gb = U50413/gi = 1621039/ug = Mm.3058/len = 3454/STRA = rev | | | | | | |
| 2 | 97169_f_at | Cluster Incl AF067058 Mus musculus clone L10 variable group of 2-cell-stage gene family mRNA, complete cds/ | 164.9 | P | 100.8 | A | 87.7 | A |
| | | cds = (166,675)/gb = AF067058/gi = 3171133/ug = Mm 35599/len = 874/STRA = for | | | | | | |
| 2 | 98840_at | Cluster Incl AF012104 Mus musculus cytoplasmic tyrosine kinase (bmx) mRNA, complete cds/cds = (8,1963)/ | 105.8 | A | 71.8 | A | 91.7 | P |
| | | gb = AF012104/gi = 2293557/ug = Mm 504/len = 2369/STRA = for | | | | | | |
| 2 | 100280_at | Cluster Incl X72805 M musculus gene for testicular hislone H1/cds = (0,626)/gb = X72805/gi = 1934959/ug = Mm 8048/ | 405.7 | P | 312.3 | P | 257.8 | P |
| | | len = 627/STRA = for | | | | | | |
| 2 | 100742_at | Cluster Incl AA409749 EST01534 Mus musculus cDNA, 3 end/clone = C0011A07/clone_end = 3/gb = AA409749/ | 237.6 | A | 107.8 | P | 116.7 | P |
| | | gi = 2066449/ug = Mm.3628/len = 337/STRA = rev | | | | | | |
| 2 | 101168_at | Cluster Incl Z31360.M. musculus (Balb/C) P/L01 mRNA/cds = (0,139)/gb = Z31360/gi = 467579/ug = Mm 5029/len = 421/ | 128.6 | P | 84.3 | P | 108.8 | A |
| | | STRA = for | | | | | | |
| 2 | 101185_at | Cluster Incl X92498 Forkhead homolog 6, (Drosophila)/cds = (277,1290)/gb = X92498/gi = 1199829/ug = Mm.5111/len = 1761/ | 534.4 | P | 277.2 | M | 447.2 | P |
| | | STRA = for | | | | | | |
| 2 | 99585_at | Cluster Incl AI894122: md85f04 x1 Mus musculus cDNA, 3 end/clone = IMAGE-375199/clone_end = 3/gb = AI894122/ | 634.9 | M | 534 | M | 684.4 | A |
| | | gi = 5600024/ug = Mm 4261/len = 425/STRA = for | | | | | | |
| 2 | 102401 _at | Cluster Incl M21085 Interferon regulatory factor 1/cds = (205,1194)/gb = M21065/gi = 198458/ug = Mm.1246/len = 2072/ | 1048.5 | P | 492.5 | A | 483.1 | A |
| | | STRA = for | | | | | | |
| 2 | 103084_at | Cluster Incl D88791 Mouse mRNA for muscle LIM protein, complete cds/cds = (0,584)/gb = D88791/gi = 1695699/ | 710 | P | 42.8 | P | 245 | P |
| | | ug = Mm 17235/len = 585/STRA = for | | | | | | |
| 2 | 103573_at | Cluster Incl D86176.Mouse mRNA for phosphatidylinositol 4-phosphate 5-kinase type I-alpha, complete cds/cds = (84,1703)/ | 109.9 | M | 80.2 | A | 102 | P |
| | | gb = D86176/gi = 1622707/ug = Mm 1945/len = 2378/STRA = for | | | | | | |
| 2 | 103854_at | Cluster Incl AW049387 UI-M-BH1-ane-d-08-0-UI s1 Mus musculus cDNA, 3 end/clone = UI-M-BH1-ane-08-0-UI/ | 196.8 | P | −16.5 | A | 78.7 | M |
| | | clone_end = 3/gb = AW049387/gi = 5909916/ug = Mm.21071/len = 335/STRA = for | | | | | | |
| 2 | 104342_i_at | Cluster Incl AI845798.UI-M-AQ1-aeb-h-07-0-UI s1 Mus musculus cDNA, 3 end/clone = UI-M-AQ1-aab-h-07-0-UI/ | 69.2 | A | −81.4 | A | −59.4 | A |
| | | clone_end = 3/gb = AI845798/gi = 5489704/ug = Mm.22526/len = 184/STRA = for | | | | | | |
| 2 | 96160_at | Cluster Incl X67056: Glycine transporter 1/cds = (240,2141)/gb = X67058/gi = 51092/ug = Mm 2834/len = 3227/STRA = for | 622.3 | A | 468.4 | A | 490.2 | P |
| 2 | 96702_at | Cluster Incl AI787317: uj66c05 x1 Mus musculus cDNA, 3 end/clone = IMAGE-1924804/clone_end = 3/gb = AI787317/ | 170.5 | A | 107.4 | A | 70.8 | A |
| | | gi = 5885993/ug = Mm 20123/len = 470/STRA = for | | | | | | |
| 2 | 92205_at | Cluster Incl AF090738: Mus musculus insulin receptor substrate-2 (Irs2) gene, partial cds/cds = (0,2206)/gb = AF090738/ | 618.4 | P | 388.8 | A | 388.1 | P |
| | | gi = 3661524/ug = Mm.34143/len = 2207/STRA = for | | | | | | |
| 2 | 92254_at | Cluster Incl M55253: Myosin Vb/cds = (46.2217)/gb = M55253/gi = 193414/ug = Mm 3536/len = 2632/STRA = for | 157 | M | −43 | P | 0 | P |
| 2 | 98290_at | Cluster Incl AF027185 Mus musculus ELP mRNA, complete cds/cds = (35,430)/gb = AF027185/gi = 4103746/ug = Mm 42090/ | 522.9 | P | 372.6 | M | 366.5 | P |
| | | len = 1081/STRA = for | | | | | | |
| 2 | 98770_at | Cluster Incl AF012708: Mus musculus centromere protein C (Cenp-c) gene, promoter region and partial cds/cds = (142,159)/ | 155.1 | P | −24 | A | 17.6 | A |
| | | gb = AF012708/gi = 2465200/ug = Mm 4649/len = 160/STRA = for | | | | | | |
| 2 | 101225_at | Cluster Incl C77257 C77257: Mus musculus cDNA, 3 end/clone = J0028804/clone_end = 3/gb = C77257/gi = 2517587/ | 477.6 | P | 221.7 | M | 189.5 | M |
| | | ug = Mm 5593/len = 501/STRA = rev | | | | | | |
| 3 | 92575_at | Cluster Incl U85489: Mus musculus Ah receptor-interacting protein (AIP) mRNA, complete cds/cds = (209,1201)/gb = U85489/ | 633.5 | P | 627.2 | P | 636.3 | P |
| | | gi = 2177173/ug = Mm 10433/len = 1367/STRA = for | | | | | | |
| 3 | 92635_at | Cluster Incl M13444 Mouse alpha-tublin isotype M-alpha-4 mRNA, complete cds/cds = (29,1375)/gb = M13444/gi = 202214/ | 649.4 | P | 637.7 | P | 548.6 | P |
| | | ug = Mm 1155/len = 1442/STRA = for | | | | | | |
| 3 | 92642_at | Cluster Incl M25944 Mouse carbonic anhydrase II (CAII) mRNA, 3 end/cds = (0,62)/gb = M25944/gi = 199078/ug = Mm 1186/ | 291.8 | P | 146.4 | P | 213.5 | P |
| | | len = 742/STRA = for | | | | | | |
| 3 | 92778_i_at | Cluster Incl Z22552: M musculus membrane glycoprotein gene/cds = (0,223)/gb = Z22552/gi = 296166/ug = Mm. 1232 | 973.5 | P | 988.8 | P | 935.3 | P |
| | | len = 224/STRA = for | | | | | | |
| 3 | 92821_at | Cluster Incl AF079565: Mus musculus ubiquitin-specific protease UBP41 (Uop41) mRNA, complete cds/cds = (0,1061)/ | 433.5 | P | 452.2 | P | 401.8 | P |
| | | gb = AF079565/gi = 3386551/ug = Mm 12914/len = 1115/STRA = for | | | | | | |
| 3 | 92836_at | Cluster Incl AA919594 vz22b07 r1 Mus musculus cDNA, 5 end/clone = IMAGE-1316437/clone_end = 5/gb = AA919594/ | 3587.3 | P | 2749.2 | P | 2793.1 | P |
| | | gi = 3066373/ug = Mm. 13097/len = 222/STRA = for | | | | | | |
| 3 | 92852_at | Cluster Incl M18194 Mouse fibronectin (FN) mRNA/cds = (0,311)/gb = M18194/gi = 193329/ug = Mm 13846/len = 905/ | 2138.2 | P | 2200.2 | P | 2124.7 | P |
| | | STRA = for | | | | | | |
| 3 | 92863_at | Cluster Incl U40826 Mus musculus WW-domain binding protein 2 mRNA, complete cds/cds = (39,824)/gb = U40826/ | 506.4 | P | 497.7 | P | 243.1 | P |
| | | gi = 1777578/ug = Mm 1405/len = 1802/STRA = for | | | | | | |
| 3 | 92885_at | Cluster Incl L48989: Troponin T3, skeletal, fast/cds = (0,719)/gb = L48989/gi = 2340049/ug = Mm 14546/len = 720/STRA = for | 3741.5 | P | 3954.9 | P | 3850.1 | P |
| 3 | 93011_at | Cluster Incl AW123904 UI-M-BH2.1-app-g-10-0-UI s1 Mus musculus cDNA, 3 end/clone = UI-M-BH2.1-app-g-10-0-UI/ | 888.6 | P | 762.6 | P | 745 | P |
| | | clone_end = 3/gb = AW123904/gi = 6099434/ug = Mm.14638/len = 433/STRA = for | | | | | | |
| 3 | 93048_at | Cluster Incl AJ005253 Mus musculus mRNA for CipP protease/cds = (32,850)/gb = AJ005253/gi = 3559934/ug = Mm.15243/ | 468.9 | P | 306.4 | P | 470.8 | P |
| | | len = 990/STRA = for | | | | | | |
| 3 | 93053_at | Cluster Incl AF068244 Mus musculus cardiac calsequesirin mRNA, complete cds/cds = (129,1376)/gb = AF068244/ | 689.7 | P | 496.9 | P | 539.2 | P |
| | | gi = 3219362/ug = Mm.15343/len = 2275/STRA = for | | | | | | |
| 3 | 93063_at | Cluster Incl U82624 Mus musculus amyloid precursor protein (APP) gene, partial cds, exons 16 and 17/cds = (0,247)/ | 3572.7 | P | 3127.3 | P | 3018 | P |
| | | gb = U82624/gi = 1778801/ug = Mm 15571/len = 248/STRA = for | | | | | | |
| 3 | 93084_at | Cluster Incl U27315 Adenine nucleotide translocator 1, skeletal muscle/cds = (86,982)/gb = U27315/gi = 902007/ | 3128.2 | P | 2484.5 | P | 2740.7 | P |
| | | ug = Mm.16228/len = 1273/STRA = for | | | | | | |
| 3 | 93086_at | Cluster Incl M18237 Mouse lg kappa chain V-region mRNA, partial cds, clone 3H9/cds = (0,321)/gb = M18237/gi = 197007/ | 275.1 | P | 247 | P | 277.8 | P |
| | | ug = Mm 16253/len = 322/STRA = for | | | | | | |
| 3 | 93284_at | Cluster Incl D78135 Mus musculus mRNA for CIRP, complete cds/cds = (82,600)/gb = D78135/gi = 1060918/ug = Mm 17898/ | 1586.2 | P | 1192.3 | P | 949.9 | P |
| | | len = 1256/STRA = for | | | | | | |
| 3 | 93330_at | Cluster Incl L02914 Aquaporin 1/cds = (59,868)/gb = L02914/gi = 193642/ug = Mm 18625/len = 2627/STRA = for | 438.5 | P | 376.8 | P | 383.9 | P |
| 3 | 93490_at | Cluster Incl AI841771 UI-M-AN0-acl-c-12-0-UI s1 Mus musculus cDNA, 3 end/clone = UI-M-AN0-acl-c-12-0-UI/ | 744.3 | P | 438.7 | P | 717.5 | P |
| | | clone_end = 3/gb = AI841771/gi = 5475984/ug = Mm 19055/len = 479/STRA = for | | | | | | |
| 3 | 93500_at | Cluster Incl M63245 Mus musculus amino levulinate synthase (ALAS-H) mRNA, 3 end/cds = (0,1369)/gb = M63245/ | 915.8 | P | 578.9 | P | 657.3 | P |
| | | gi = 1220401/ug = Mm 19143/len = 1448/STRA = for | | | | | | |
| 3 | 93511_at | Cluster Incl L38971 Integral membrane protein 2/cds = (147,938)/gb = L38971/gi = 624777/ug = Mm 193/len = 1635/ | 3408.4 | P | 2934.1 | P | 3557.6 | P |
| | | STRA = for | | | | | | |
| 3 | 93540_at | Cluster Incl L13290 Mus musculus ADP-ribosylarginine hydrolase mRNA, complete cds/cds = (0,1088)/gb = L13290/ | 671.1 | P | 613.4 | P | 501.9 | P |
| | | gi = 402489/ug = Mm 20047/len = 1089/STRA = for | | | | | | |
| 3 | 93732_f_at | Cluster Incl AF104358.Mus musculus synectin mRNA, complete cds/cds = (72,1073)/gb = AF104358/gi = 4219017/ | 946 | P | 827.3 | P | 794.6 | P |
| | | ug = Mm 20945/len = 1600/STRA = for | | | | | | |
| 3 | 93736_at | Cluster Incl AF090686: Mus musculus transcobalmin II (Tcn2) mRNA, complete cds/cds = (127,1419)/gb = AF090686/ | 360.6 | P | 432.4 | P | 310.3 | P |
| | | gi = 3659876/ug = Mm.20948/len = 1878/STRA = for | | | | | | |
| 3 | 93767_i_at | Cluster Incl AW124052 UI-M-BH2.1-apn-e-06-0-UI s1 Mus musculus cDNA, 3 end/clone = UI-M-BH2.1-apn-e-06-0-UI/ | 2398.1 | P | 2292.9 | P | 2349.3 | P |
| | | clone_end = 3/gb = AW124052/gi = 6099582/ug = Mm 21183/len = 246/STRA = for | | | | | | |
| 3 | 93780_at | Cluster Incl AW060827 UI-M-BH1-amx-b-12-0-UI s1 Mus musculus cDNA, 3 end/clone = UI-M-BH1-amx-b-12-0-UI/ | 544.7 | P | 529.2 | P | 508.9 | P |
| | | clone_end = 3/gb = AW060827/gi = 6008578/ug = Mm 2125/len = 567/STRA = for | | | | | | |
| 3 | 93966_at | Cluster Incl AI849706 UI-M-AL1-ahl-h-11-0-UI s1 Mus musculus cDNA, 3 end/clone = UI-M-AL1-ahl-h-11-0-UI/ | 832 | P | 736.6 | P | 776.7 | P |
| | | clone_end = 3/gb = AI849706/gi = 5493612/ug = Mm 21634/len = 217/STRA = for | | | | | | |
| 3 | 94022_at | Cluster Incl AI844754 UI-M-AL1-ahq-e-04-0-UI s1 Mus musculus cDNA, 3 end/clone = UI-M-AL1-ahq-e-04-0-UI/ | 1472.4 | P | 1361.4 | P | 1628.5 | P |
| | | clone_end = 3/gb = AI844754/gi = 5488660/ug = Mm 21859/len = 290/STRA = for | | | | | | |
| 3 | 94244_at | Cluster Incl AW123807: UI-M-BH2.1-apm-f-02-0-UI s1 Mus musculus cDNA, 3 end/clone = UI-M-BH2.1-apm-f-02-0-UI/ | 794.8 | P | 654.7 | P | 657 | P |
| | | clone_end = 3/gb = AW123807/gi = 6099337/ug = Mm 22360/len = 226/STRA = for | | | | | | |
| 3 | 94248_at | Cluster Incl M62419 Mouse clathrin-associated protein (AP47) mRNA, complete cds/cds = (72,1343)/gb = M62419/ | 840.5 | P | 807.4 | P | 668 | P |
| | | gi = 191985/ug = Mm 2237/len = 1966/STRA = for | | | | | | |
| 3 | 94290_at | Cluster Incl AW124346: UI-M-BH2.1-apq-g-05-0-UI s1 Mus musculus cDNA, 3 end/clone = UI-M-BH2.1-apq-g-05-0-UI/ | 494.2 | P | 391.4 | P | 479.6 | P |
| | | clone_end = 3/gb = AW124346/gi = 6099876/ug = Mm 22577/len = 235/STRA = for | | | | | | |
| 3 | 94833_at | Cluster Incl M91380: Follistatin-like/cds = (79,999)/gb = M91380/gi = 349005/ug = Mm 22763/len = 2823/STRA = for | 2317 | P | 2197.4 | P | 2377.8 | P |
| 3 | 94842_at | Cluster Incl AI853630 UI-M-BH0-ajp-h-03-0-UI s1 Mus musculus cDNA, 3 end/clone = UI-M-BH0-ajp-h-03-0-UI/ | 2470.2 | P | 1746.2 | P | 2408.4 | P |
| | | clone_end = 3/gb = AI853630/gi = 5497536/ug = Mm 22876/len = 547/STRA = for | | | | | | |
| 3 | 94920_at | Cluster Incl AI854028: UI-M-BH0-ajm-o-01-UI.s1 Mus musculus cDNA, 3 end/clone = UI-M-BH0-ajm-c-01-0-UI/ | 425.2 | P | 350.9 | P | 359.2 | P |
| | | clone_end = 3/gb = AI854028 = 5497934/ug = Mm.24187/len = 439/STRA = for | | | | | | |
| 3 | 95449_at | Cluster Incl AW049793: UI-M-BH1-anm-f-04-0-UI.s1 Mus musculus cDNA, 3 end/clone = UI-M-BH1-anm-f-04-0-UI/ | 495.6 | P | 450 | P | 466.1 | P |
| | | clone_end = 3/gb = AW049793/gi = 5910322/ug = Mm.24635/len = 569/STRA = for | | | | | | |
| 3 | 95518_at | Cluster Incl AW122893: UI-M-BH2-apa-d-07-0-UI s1 Mus musculus cDNA, 3 end/clone = UI-M-BH2.1-apa-d-07-0-UI/ | 1567.5 | P | 1292.6 | P | 1421 | P |
| | | clone_end = 3/gb = AW122893/gi = 6098423/ug = Mm 25311/len = 217/STRA = for | | | | | | |
| 3 | 96037_at | Cluster Incl X61454 M. musculus mRNA for I3 clone/cds = (0,224)/gb = X61454/gi = 51477/ug = Mm 2585/len = 449/STRA = for | 936.3 | P | 963.3 | P | 868 | P |
| 3 | 96082_at | Cluster Incl AI850644 UI-M-BG1-aik-f-01-0-UI s1 Mus musculus cDNA, 3 end/clone = UI-M-BG1-aik-f-01-0-UI/ | 832.8 | P | 823.3 | P | 856.2 | P |
| | | clone_end = 3/gb = AI850644/gi = 5494550/ug = Mm 26614/len = 374/STRA = for | | | | | | |
| 3 | 96614_at | Cluster Incl AA783368 vw52e04 r1 Mus musculus cDNA, 5 end/clone = IMAGE-1247550/clone_end = 5/gb = AA763388/ | 231.3 | P | 199.7 | P | 255.8 | P |
| | | gi = 2813115/ug = Mm 27112/len = 450/STRA = for | | | | | | |
| 3 | 96615_at | Cluster Incl AI840137 UI-M-AO0-acc-b-01-UI s1 Mus musculus cDNA, 3 end/clone = UI-M-AO0-acc-b-07-0-UI/ | 1257.5 | P | 1083 | P | 925.9 | P |
| | | clone_end = 3/gb = AI840137/gi = 5474350/ug = Mm 27114/len = 390/STRA = for | | | | | | |
| 3 | 96662_at | Cluster Incl AI847054 UI-M-AP1-agn-a-07-0-UI s1 Mus musculus cDNA, 3 end/clone = UI-M-AP1-agn-a-07-0-UI/ | 408.6 | P | 273.6 | P | 455.5 | P |
| | | clone_end = 3/gb = AI847054/gi = 5490960/ug = Mm 27363/len = 389/STRA = for | | | | | | |
| 3 | 97235_f_at | Cluster Incl AW124988 UI-M-BH2 1-apv-d-12-0-UI s1 Mus musculus cDNA, 3 end/clone = UI-M-BH2.1-apv-d-12-0-UI/ | 507.2 | P | 420.6 | P | 475.5 | P |
| | | clone_end = 3/gb = AW124988/gi = 6100518/ug = Mm 27822/len = 353/STRA = for | | | | | | |
| 3 | 97800_at | Cluster Incl AI837308 UI-M-AK0-adf-a-09-0-UI s1 Mus musculus cDNA, 3 end/clone = UI-M-AK0-adf-a-09-0-UI/ | 1607.5 | P | 1492.3 | P | 1269.1 | P |
| | | clone_end = 3/gb = AI837308/gi = 5471521/ug = Mm.28123/len = 287/STRA = for | | | | | | |
| 3 | 98908_at | Cluster Incl AW125510 UI-M-BH2-aqm-e-12-0-UI s1 Mus musculus cDNA, 3 end/clone = UI-M-BH2-aqm-e-12-0-UI/ | 1506.9 | P | 1496.7 | P | 1053.8 | P |
| | | clone_end = 3/gb = AW125510/gi = 6101040/ug = Mm 28586/len = 389/STRA = for | | | | | | |
| 3 | 94501_at | Cluster Incl AI835784 UI-M-AI0-aaq-b-10-0-UI s1 Mus musculus cDNA, 3 end/clone = UI-M-AI0-aaq-b-10-0-UI/ | 1775.3 | P | 1867.7 | P | 1654.3 | P |
| | | clone_end = 3/gb = AI835784/gi = 5469997/ug = Mm 28948/len = 395/STRA = for | | | | | | |
| 3 | 95084_f_at | Cluster Incl AI844995 UI-M-BG0-ahu-e-03-0-UI s1 Mus musculus cDNA, 3 end/clone = UI-M-BG0-ahu-e-03-0-UI/ | 530.1 | P | 429.1 | P | 423.6 | P |
| | | clone_end = 3/gb = AI844995/gi = 5488901/ug = Mm 29256/len = 218/STRA = for | | | | | | |
| 3 | 95117_at | Cluster Incl U04710 Insulin-like growth factor 2 receptor/cds = (132,7580)/gb = U04710/gi = 451552/ug = Mm 2938/len = 8877/ | 275.4 | P | 146.2 | P | 143.7 | P |
| | | STRA = for | | | | | | |
| 3 | 95139_at | Cluster Incl AW122075 UI-M-BH2-aoo-f-04-0-UI s1 Mus musculus cDNA, 3 end/clone = UI-M-BH2-aoo-f-04-0-UI/ | 562.2 | P | 395.5 | P | 318.5 | P |
| | | clone_end = 3/gb = AW122075/gi = 6097482/ug = Mm 29432/len = 463/STRA = for | | | | | | |
| 3 | 95146_at | Cluster Incl AI839280: UI-M-AO0-acb-b-06-UI s1 Mus musculus cDNA, 3 end/clone = UI-M-AO0-acb-b-06-0-UI/ | 541.2 | P | 489.5 | P | 511.3 | P |
| | | clone_end = 3/gb = AI839280/gi = 5473493/ug = Mm 29455/len = 265/STRA = for | | | | | | |
| 3 | 95683_g_at | Cluster Incl AB026432 Mus musculus mRNA for XPE UV-damaged DNA binding factor, complete cds/cds = (81,3503)/ | 2126.6 | P | 1536.7 | P | 2084.1 | P |
| | | gb = AB026432/gi = 5931595/ug = Mm.29623/len = 4171/STRA = for | | | | | | |
| 3 | 96255_at | Cluster Incl AF067395 Mus musculus NIX (Nix) mRNA, nuclear gene encoding mitochondrial protein, complete cds/ | 853.5 | P | 959.9 | P | 928.7 | P |
| | | cds = (99,755)/gb = AF067395/gi = 138823/ug = Mm 29820/len = 1285/STRA = for | | | | | | |
| 3 | 96267_at | Cluster Incl AI846127 UI-M-AN1-afj-b-05-0-UI s2 Mus musculus cDNA, 3 end/clone = UI-M-AN1-afj-b-05-0-UI/ | 3405.1 | P | 3127.4 | P | 3487.3 | P |
| | | clone_end = 3/gb = AI846127/gi = 5490033/ug = Mm 29842/len = 417/STRA = for | | | | | | |
| 3 | 96344_at | Cluster Incl X61600 M musculus gene for beta-enolase/cds = (66,1370)/gb = X61600/gi = 50848/ug = Mm 29994/len = 1450/ | 3943.7 | P | 3389.3 | P | 4555.8 | P |
| | | STRA = for | | | | | | |
| 3 | 96351_at | Cluster Incl AI846097 UI-M-AK1-aew-c-06-0-UI s1 Mus musculus cDNA, 3 end/clone = UI-M-AK1-aew-c-06-0-UI/ | 499.5 | P | 384.1 | P | 245.4 | P |
| | | clone_end = 3/gb = AI846097/gi = 5489968/ug = Mm 30001/len = 183/STRA = for | | | | | | |
| 3 | 96854_at | Cluster Incl AJ010391 Mus musculus copa gene, partial/cds = (0,94)/gb = AJ010391/gi = 5763780/ug = Mm 30041/len = 95/ | 1025.3 | P | 933.8 | P | 921.1 | P |
| | | STRA = for | | | | | | |
| 3 | 96916_at | Cluster Incl AW046793 UI-M-BH1-alz-b-05-0-UI.s1 Mus musculus cDNA, 3 end/clone = UI-M-BH1-alz-b-05-0-UI/ | 748.9 | P | 745.5 | P | 697.7 | P |
| | | clone_end = 3/gb = AW046793/gi = 5907322/ug = Mm 30152/len = 237/STRA = for | | | | | | |
| 3 | 96954_at | Cluster Incl AW122567 UI-M-BH2-2-aox-f-01-0-UI s1 Mus musculus cDNA, 3 end/clone = UI-M-BH2 2-aox-f-01-0-UI/ | 683.8 | P | 674.5 | P | 479 | P |
| | | clone_end = 3/gb = AW122567/gi = 6098092/ug = Mm 30212/len = 391/STRA = for | | | | | | |
| 3 | 96956_at | Cluster Incl AW125526 UI-M-BH2.2-aqm-g-04-0-UI.s1 Mus musculus cDNA, 3 end/clone = UI-M-BH2.2-aqm-g-04-0-UI/ | 2772.4 | P | 2315.8 | P | 2662.7 | P |
| | | clone_end = 3/gb = AW125526/gi = 6101058/ug = Mm 30215/len = 443/STRA = for | | | | | | |
| 3 | 97447_at | Cluster Incl AI836718 UI-M-AJ0-aax-d-11-0-UI.s1 Mus musculus cDNA, 3 end/clone = UI-M-AJ0-aax-d-11-0-UI/ | 1271.8 | P | 906.4 | P | 1445.7 | P |
| | | clone_end = 3/gb = AI836718/gi = 5470931/ug = Mm 30244/len = 276/STRA = for | | | | | | |
| 3 | 97509_f_at | Cluster Incl U22324 Mus musculus fibroblast growth factor receptor-1 mRNA, long isoform precursor, complete cds/ | 685.2 | P | 475.8 | P | 663 | P |
| | | cds = (57,2525)/gb = U22324/gi = 722340/ug = Mm 3157/len = 2526/STRA = for | | | | | | |
| 3 | 97526_at | Cluster Incl AW123294: UI-M-BH2 1-apg-h-02-0-UI s1 Mus musculus cDNA, 3 end/clone = UI-M-BH2 1-apg-h-02-0-UI/ | 489.8 | P | 286.8 | P | 160.2 | P |
| | | clone_end = 3/gb = AW123294/gi = 6098824/ug = Mm 3256/len = 380/STRA = for | | | | | | |
| 3 | 98042_at | Cluster Incl AW049787 UI-M-BH1-anm-e-03-0-UI s1 Mus musculus cDNA, 3 end/clone = UI-M-BH1-anm-e-03-0-UI/ | 540.9 | P | 470.7 | P | 328.5 | P |
| | | clone_end = 3/gb = AW049787/gi = 5910316/ug = Mm 34126/len = 434/STRA = for | | | | | | |
| 3 | 98097_r_at | Cluster Incl AI840219: UI-M-AI0-aal-g-03-0-UI s2 Mus musculus cDNA, 3 end/clone = UI-M-Al0-aal-g-03-0-UI/ | 114.8 | P | 106.2 | P | 140.1 | P |
| | | gb = AI840219/gi = 5474432/ug = Mm.34708/len = 371/STRA = for | | | | | | |
| 3 | 98126_s_at | Cluster Incl X67140: Atpase, Ca++ transporting, cardiac muscle, fast twitch 1/cds = (0,548)/gb = X67140/gi = 509146/ | 3328.3 | P | 3442.6 | P | 4056.6 | P |
| | | ug = Mm 35134/len = 855/STRA = for | | | | | | |
| 3 | 98518_f_at | Cluster Incl AI604345 vv70f10.x1 Mus musculus cDNA, 3 end/clone = IMAGE-1227787/clone_end = 3/gb = AI604345/ | 5878.3 | P | 6161.4 | P | 5917.3 | P |
| | | gi = 4613512/ug = Mm.35806/len = 397/STRA = for | | | | | | |
| 3 | 98519_r_at | Cluster Incl AI604345 vv70f10 x1 Mus musculus cDNA, 3 end/clone = IMAGE-1227787/clone_end = 3/gb = AI604345/ | 1306.3 | P | 1050.8 | P | 1106.5 | P |
| | | gi = 4613512/ug = Mm.35806/len = 397/STRA = for | | | | | | |
| 3 | 98624_at | Cluster Incl X75316 M musculus seb4 mRNA/cds = (235,948)/gb = X75316/gi = 407467/ug = Mm.3865/len = 1908/STRA = for | 567 | P | 425.4 | P | 4712 | P |
| 3 | 98630_at | Cluster Incl AW106745 um33b04 y1 Mus musculus cDNA, 5 end/clone = IMAGE-2236303/clone_end = 5/gb = AW106745/ | 437.5 | P | 412.4 | P | 402.8 | P |
| | | gi = 6077545/ug = Mm 38792/len = 600/STRA = for | | | | | | |
| 3 | 99103_at | Cluster Incl AF036341 Mus musculus interferon regulatory factor-3 (IRF3) gene, partial cds/cds = (0,247)/gb = AF036341/ | 273.8 | P | 235.8 | P | 290.5 | P |
| | | gi = 2674204/ug = Mm 3960/len = 248/STRA = for | | | | | | |
| 3 | 99142_at | Cluster Incl AI834895: UI-M-ANI-afe-d-08-0-UI.s1 Mus musculus cDNA, 3 end/clone = UI-M-AN1-afe-d-08-0-UI/ | 749.3 | P | 823.9 | P | 739.8 | P |
| | | clone_end = 3/gb = AI834895/gi = 5469108/ug = Mm 41405/len = 436/STRA = for | | | | | | |
| 3 | 99153_at | Cluster Incl AI854607: UI-M-BH0-ake-c-10-0-UI.s1 Mus musculus cDNA, 3 end/clone = UI-M-BH0-ake-c-10-0-UI/ | 295.7 | P | 202.3 | P | 338.5 | P |
| | | clone_end = 3/gb = AI854607/gi = 5498513/ug = Mm 41574/len = 489/STRA = for | | | | | | |
| 3 | 99161_at | Cluster Incl AI843359: UI-M-AQ1-adz-d-05-0-UI s1 Mus musculus cDNA, 3 end/clone = UI-M-AQ1-adz-d-05-0-UI/ | 1625.4 | P | 1487.4 | P | 1603.1 | P |
| | | clone_end = 3/gb = AI843359/gi = 5477572/ug = Mm 41665/len = 425/STRA = for | | | | | | |
| 3 | 99188_at | Cluster Incl AA615161 vn96a04 r1 Mus musculus cDNA, 5 end/clone = IMAGE-1039758/clone_end = 5/gb = AA615161/ | 830.6 | P | 618 | P | 652.6 | P |
| | | gi = 2502389/ug = Mm 41890/len = 495/STRA = for | | | | | | |
| 3 | 99667_at | Cluster Incl U08439 Mus musculus Balb/c cytochrome c oxidase subunit VIaH mRNA, complete cds/cds = (57,350)/ | 1101 | P | 985.4 | P | 1160.9 | P |
| | | gb = U08439/gi = 475704/ug = Mm.43824/len = 466/STRA = for | | | | | | |
| 3 | 99675_at | Cluster Incl AI839766 UI-M-AN0-acq-e-09-0-UI.s1 Mus musculus cDNA, 3 end/clone = UI-M-AN0-acq-e-09-0-UI/ | 366.1 | P | 288.8 | P | 279.5 | P |
| | | clone_end = 3/gb = AI839766/gi = 5474019/ug = Mm 44202/len = 366/STRA = for | | | | | | |
| 3 | 100057_at | Cluster Incl AI838320 UI-M-AO0-aby-f-05-0-UI s1 Mus musculus cDNA, 3 end/clone = UI-M-AO0-aby-f-05-0-UI/ | 402.2 | P | 339.8 | P | 215 | P |
| | | clone_end = 3/gb = AI838320/gi = 5472533/ug = Mm 4467/len = 529/STRA = for | | | | | | |
| 3 | 100084_at | Cluster Incl X60671 M musculus mRNA for ezrin/cds = (95,1855)/gb = X60671/gi = 50880/ug = Mm 4451/len = 2701/ | 1325.6 | P | 1223.9 | P | 1021.1 | P |
| | | STRA = for | | | | | | |
| 3 | 100571_at | Cluster Incl AW123934 UI-M-BH2.3-aqc-b-11-0-UI s1 Mus musculus cDNA, 3 end/clone = UI-M-BH2 3-aqc-b-11-0-UI/ | 844.3 | P | 766.2 | P | 725.9 | P |
| | | clone_end = 3/gb = AW123934/gi = 6099464/ug = Mm 5856/len = 237/STRA = for | | | | | | |
| 3 | 100574_f_at | Cluster Incl L09104 Glucose phosphate isomerase 1 complex/cds = (2066,2938)/gb = L09104/gi = 309267/ug = Mm 589/ | 4653.7 | P | 4137.4 | P | 4759.8 | P |
| | | len = 3194/STRA = for | | | | | | |
| 3 | 100600_at | Cluster Incl M58661 CD24a antigen/cds = (72,302)/gb = M58661/gi = 198985/ug = Mm 6417/len = 1800/STRA = for | 4291.6 | P | 3911.3 | P | 4176.1 | P |
| 3 | 100630_f_at | Cluster Incl AA241764.mw26g09 r1 Mus musculus cDNA, 5 end/clone = IMAGE-671872/clone_end = 5/gb = AA241764/ | 2201.9 | P | 1606.3 | P | 1667.7 | P |
| | | gi = 1872796/ug = Mm 667/len = 240/STRA = for | | | | | | |
| 3 | 101029_f_at | Cluster Incl M15501 Actin, alpha, cardiac/cds = (0,1127)/gb = M15501/gi = 191653/ug = Mm 686/len = 1226/STRA = for | 5071.4 | P | 3754.6 | P | 4230.8 | P |
| 3 | 101047_at | Cluster Incl AW123697 UI-M-BH2 1-apl-c-11-0-UI s1 Mus musculus cDNA, 3 end/clone = UI-M-BH2 1-apl-c-11-0-UI/ | 1998.9 | P | 1437.9 | P | 1792.6 | P |
| | | clone_end = 3/gb = AW123697/gi = 6099227/ug = Mm.7/len = 413/STRA = for | | | | | | |
| 3 | 101055_at | Cluster Incl J05261 Protective protein for beta-galactosidase/cds = (213,1637)/gb = J05261/gi = 200492/ug = Mm 7046/ | 1116.2 | P | 863.1 | P | 978.4 | P |
| | | len = 1987/STRA = for | | | | | | |
| 3 | 101102_at | Cluster Incl AJ223156 Mus musculus alpha4 gene, exon2 and join CDS/cds = (0,831)/gb = AJ223156/gi = 3127824/ | 472.8 | P | 517 | P | 462.2 | P |
| | | ug = Mm 7454/len = 1132/STRA = for | | | | | | |
| 3 | 101109_at | Cluster Incl U43512 Dystroglycan 1/cds = (0,825)/gb = U43512/gi = 1155350/ug = Mm 7524/len = 3170/STRA = for | 1372.4 | P | 1048.7 | P | 1128.9 | P |
| 3 | 101517_at | Cluster Incl X81058: M musculus tex261 mRNA/cds = (35,625)/gb = X81058/gi = 531527/ug = Mm 848/len = 1398/STRA = for | 674 | P | 775.7 | P | 717.8 | P |
| 3 | 101973_at | Cluster Incl Y15163 Mus musculus mRNA for mrg1 protein/cds = (219,1028)/gb = Y15163/gi = 3059126/ug = Mm.9524/ | 760.8 | P | 609.2 | P | 657.8 | P |
| | | len = 1943/STRA = for | | | | | | |
| 3 | 101995_at | Cluster Incl U40930 Mus musculus oxidative stress-induced protein mRNA, complete cds/cds = (33,1361)/gb = U40930/ | 1022.2 | P | 891.5 | P | 1132.2 | P |
| | | gi = 1280523/ug = Mm 9846/len = 2000/STRA = for | | | | | | |
| 3 | 102024_at | Cluster Incl AF000581 Mus musculus p300/CBP/Co-Integrator protein mRNA, complete cds/cds = (109,4305)/ | 96.7 | P | 53.7 | P | 100.6 | P |
| | | gb = AF000581/gi = 2934905/ug = Mm 1011/len = 4609/STRA = for | | | | | | |
| 3 | 102043_at | Cluster Incl AF047704 Mus musculus tuftelin mRNA, complete cds/cds = (22,1194)/gb = AF047704/gi = 2920655/ | 590.2 | P | 630.1 | P | 513.4 | P |
| | | ug = Mm 10214/len = 2580/STRA = for | | | | | | |
| 3 | 102063_at | Cluster Incl AF079535: Mus musculus 3-phosphoinositide dependent protein kinase-1 mRNA, complete cds/cds = (91,1770)/ | 280.8 | P | 307.7 | P | 202.9 | P |
| | | gb = AF079535/gi = 4028126/ug = Mm 10504/len = 1923/STRA = for | | | | | | |
| 3 | 102336_at | Cluster Incl AF060565: Mus musculus RW1 protein mRNA, complete cds/cds = (28,5517)/gb = AF060565/gi = 3091277/ | 653.5 | P | 643.4 | P | 565.7 | P |
| | | ug = Mm 10808/len = 6271/STRA = for | | | | | | |
| 3 | 102382_at | Cluster Incl AB014494: Aryl hydrocarbon receptor nuclear translocator-like/cds = (406,2286)/gb = AB014494/gi = 3402482/ | 88.5 | P | 136.2 | P | 104.1 | P |
| | | ug = Mm.12177/len = 2792/STRA = for | | | | | | |
| 3 | 102402_at | Cluster Incl AJ001261 Mus musculus mRNA for NIPSNAP2 protein/cds = (48,893)/gb = AJ001261/gi = 2769257/ | 1255.9 | P | 1267.6 | P | 1245.3 | P |
| | | ug = Mm.12468/len = 1604/STRA = for | | | | | | |
| 3 | 102413_at | Cluster Incl AW124311 UI-M-BH2.1-apq-d-04-0-UI.s1 Mus musculus cDNA, 3 end/clone = UI-M-BH2.1-apq-d-04-0-UI/ | 1016 | P | 865.7 | P | 1000.3 | P |
| | | clone_end = 3/gb = AW124311/gi = 6099841/ug = Mm 12607/len = 222/STRA = for | | | | | | |
| 3 | 102426_at | Cluster Incl U93291 Mus musculus skeletal muscle calsequestrin mRNA, complete cds/cds = (179,1378)/gb = U93291/ | 1747.1 | P | 1496.5 | P | 1820.1 | P |
| | | gi = 1931626/ug = Mm 12829/len = 1395/STRA = for | | | | | | |
| 3 | 102797_at | Cluster Incl X95281: Mus musculus mRNA for retinal short-chain dehydrogenase/reductase/cds = (416,1324)/gb = X95281/ | 383 | P | 423.5 | P | 290.8 | P |
| | | gi = 3776074/ug = Mm 14063/len = 1754/STRA = for | | | | | | |
| 3 | 103094_at | Cluster Incl AI036894 uh20g09.r1 Mus musculus cDNA, 5 end/clone = IMAGE-1746016/clone_end = 5/gb = AI036894/ | 514.8 | P | 415.3 | P | 381.1 | P |
| | | gi = 3260585/ug = Mm.1749/len = 163/STRA = for | | | | | | |
| 3 | 103322_at | Cluster Incl L35034 Ribose 5-phosphate isomerase A/cds = (207,917)/gb = L35034/gi = 836673/ug = Mm 17905/len = 1763/ | 983.9 | P | 1032.1 | P | 529.2 | P |
| | | STRA = for | | | | | | |
| 3 | 103412_at | Cluster Incl AI847230.UI-M-AI1-afq-0-12-0-UI s1 Mus musculus CDNA, 3 end/clone = UI-M-AI1-afq-c-12-0-UI/clone_end = 3/ | 210.2 | P | 202.4 | P | 243.6 | P |
| | | gb = AI847230/gi = 5491136/ug = Mm 18830/len = 459/STRA = for | | | | | | |
| 3 | 103581_at | Cluster Incl Y14004.Mus musculus mRNA for acyl-CoA thioesterase/cds = (8,1267)/gb = Y14004/gi = 2765361/ug = Mm.1978/ | 622.8 | P | 575.1 | P | 602.8 | P |
| | | len = 1574/STRA = for | | | | | | |
| 3 | 103623_at | Cluster Incl L39790 Mus musculus fibrillin 2 (fbn2) gene, complete cds/cds = (0,8723)/gb = L39790/gi = 762830/ | 848.3 | P | 420.2 | P | 486.8 | P |
| | | ug = Mm 20271/len = 8724/STRA = for | | | | | | |
| 3 | 103644_at | Cluster Incl D13139.Dipeptidase 1 (renal)/cds = (257,1489)/gb = D13139/gi = 220397/ug = Mm 20388/len = 1655/STRA = for | 277.8 | P | 219.8 | P | 270.5 | P |
| 3 | 103684_f_at | Cluster Incl AA959648 vw57102 s1 Mus musculus cDNA, 3 end/clone = IMAGE-1247931/clone_end = 3/gb = AA959648/ | 275.1 | P | 224.5 | P | 213.2 | P |
| | | gi = 3125548/ug = Mm.20693/len = 426/STRA = for | | | | | | |
| 3 | 103675_at | Cluster Incl Y17793 Mus musculus mRNA for Dutt1 protein/cds = (253,5091)/gb = Y17793/gi = 3688435/ug = Mm.20832/ | 92.4 | P | 140.2 | P | 42.6 | P |
| | | len = 6922/STRA = for | | | | | | |
| 3 | 103807_at | Cluster Incl AB012265: Mus musculus mRNA for wizl, complete cds/cds = (772,5457)/gb = AB012265/gi = 3551181/ | 479.7 | P | 331.6 | P | 344.1 | P |
| | | ug = Mm.20878/len = 6425/STRA = rev | | | | | | |
| 3 | 103809_r_at | Cluster Incl AF063229: Dynein, cyctoplasmic, intermediate chain 1/cds = (141,2027)/gb = AF063229/gi = 3493586/ | 911.1 | P | 976.6 | P | 835 | P |
| | | ug = Mm 20893/len = 2586/STRA = for | | | | | | |
| 3 | 103826_at | Cluster Incl AF086830 Mus musculus leukemia/lymphoma related factor LRF (Lrf) mRNA, complete cds/cds = (58,1755)/ | 812.9 | P | 747.4 | P | 867.5 | P |
| | | gb = AF086830/gi = 3599510/ug = Mm 20920/len = 1793/STRA = for | | | | | | |
| 3 | 103904_at | Cluster Incl X81584 M musculus mRNA for Insulin-like growth factor binding protein-6/cds = (192,908)/gb = X81584/ | 491.4 | P | 561.1 | P | 687.4 | P |
| | | gi = 550386/ug = Mm.21373/len = 1138/STRA = for | | | | | | |
| 3 | 104044_at | Cluster Incl AI853663 UI-M-BH0-ajq-f-08-0-UI s1 Mus musculus cDNA, 3 end/clone = UI-M-BH0-ajq-f-08-0-UI/ | 730 | P | 599.1 | P | 552.1 | P |
| | | clone_end = 3/gb = AI853663/gi = 5497609/ug = Mm.21480/len = 432/STRA = for | | | | | | |
| 3 | 104059_at | Cluster Incl AI851751 UI-M-BH0-aim-c-03-0-UI.s1 Mus musculus cDNA, 3 end/clone = UI-M-BH0-aim-c-03-0-UI/ | 411.3 | P | 449.3 | P | 306.4 | P |
| | | clone_end = 3/gb = AI851751/gi = 5495657/ug = Mm.21553/len = 377/STRA = for | | | | | | |
| 3 | 104082_at | Cluster Incl AI848325 UI-M-AH1-ago-b-05-0-UI s1 Mus musculus cDNA, 3 end/clone = UI-M-AH1-ago-b-05-0-UI | 810.3 | P | 679.6 | P | 689.5 | P |
| | | clone_end = 3/gb = AI848325/gi = 5492231/ug = Mm 21759/len = 559/STRA = for | | | | | | |
| 3 | 104140_s_at | Cluster Incl AI846989 UI-M-AP1-agi-b-11-0-UI s1 Mus musculus cDNA, 3 end/clone = UI-M-AP1-agi-b-11-0-UI/ | 949.1 | P | 1075 | P | 1311.8 | P |
| | | clone_end = 3/gb = AI846989/gi = 5490895/ug = Mm.22121/len = 137/STRA = for | | | | | | |
| 3 | 104141_at | Cluster Incl AI850401 UI-M-BG1-aig-f-01-0-UI s1 Mus musculus cDNA, 3 end/clone = UI-M-BG1-aig-f-01-0-UI/ | 726.3 | P | 781.1 | P | 752.9 | P |
| | | clone_end = 3/gb = AI850401/gi = 5494307/ug = Mm.22123/len = 511/STRA = for | | | | | | |
| 3 | 104148_at | Cluster Incl AA939571: vz51d08 r1 Mus musculus cDNA, 5 end/clone = IMAGE.1329999/clone_end = 5/gb = AA939571/ | 840.9 | P | 714.6 | P | 544.5 | P |
| | | gi = 3100348/ug = Mm 22183/len = 420/STRA = for | | | | | | |
| 3 | 104314_at | Cluster Incl AI851206 UI-M-BH0-aix-f-05-0-UI s1 Mus musculus cDNA, 3 end/clone = UI-M-BH0-ajx-f-05-0-UI/clone_end = 3/ | 776.8 | P | 812.4 | P | 907.3 | P |
| | | gb = AI851206/gi = 5495112/ug = Mm.22405/len = 405/STRA = for | | | | | | |
| 3 | 104331_at | Cluster Incl AA832935: uc93c01 r1 Mus musculus cDNA, 5 end/clone = IMAGE-1433184/clone_end = 5/gb = AA832935/ | 1229 | P | 1200.2 | P | 925.2 | P |
| | | gi = 2906863/ug = Mm.22478/len = 391/STRA = for | | | | | | |
| 3 | 104360_at | Cluster Incl AI841844 UI-M-AO0-acb-g-01-0-UI s2 Mus musculus cDNA, 3 end/clone = UI-M-AO0-acb-g-01-0-UI/ | 1517.6 | P | 1140.7 | P | 1217.3 | P |
| | | clone_end = 3/gb = AI841844/gi = 5476057/ug = Mm.22588/len = 507/STRA = for | | | | | | |
| 3 | 104363_at | Cluster Incl AA763213: vv88f01.r1 Mus musculus cDNA, 5 end/clone = IMAGE-1229497/clone_end = 5/gb = AA763213/ | 3584 | P | 3944.4 | P | 4372 | P |
| | | gi = 2812960/ug = Mm.22608/len = 309/STRA = rev | | | | | | |
| 3 | 104366_at | Cluster Incl AW047831 UI-M-BH1-ais-c-04-0-UI s1 Mus musculus cDNA, 3 end/clone = UI-M-BH1-als-c-04-0-UI/ | 607.7 | P | 441.1 | P | 366.5 | P |
| | | clone_end = 3/gb = AW047831/gi = 5908360/ug = Mm.22616/len = 200/STRA = for | | | | | | |
| 3 | 104555_at | Cluster Incl AA733946: vv21f05 r1 Mus musculus cDNA, 5 end/clone = IMAGE-1223073/clone_end = 5/gb = AA733946/ | 1815.9 | P | 1062.7 | P | 1534 | P |
| | | gi = 2755613/ug = Mm.23786/len = 313/STRA = for | | | | | | |
| 3 | 104607_at | Cluster Incl AF093624 Mus musculus Nap-like 1 protein (Nspl 1) gene, complete cds, tRNA-Sec gene, complete sequence, | 704.9 | P | 424.6 | P | 651.7 | P |
| | | and FosB protein (Fosb) gene, complete cds/cds = (164,1579)/gb = AF093624/gi = 4235385/ug = Mm.24142/len = 1938/ | | | | | | |
| | | STRA = for | | | | | | |
| 3 | 104614_at | Cluster Incl AI852765 UI-M-BH0-aly-c-05-0-UI s1 Mus musculus cDNA, 3 end/clone = UI-M-BH0-aly-c-05-0-UI/ | 1614.3 | P | 1438.1 | P | 1707.4 | P |
| | | clone_end = 3/gb = AI852765/gi = 5496671/ug = Mm.24193/len = 401/STRA = for | | | | | | |
| 3 | 104709_at | Cluster Incl AI843665 UI-M-AK1-aer-a-11-0-UI s1 Mus musculus cDNA, 3 end/clone = UI-M-AK1-aer-a-11-0-UI/ | 544.1 | P | 537.4 | P | 523.2 | P |
| | | clone_end = 3/gb = AI843665/gi = 5477878/ug = Mm.24426/len = 314/STRA = for | | | | | | |
| 3 | 104760_at | Cluster Incl AI838527 UI-M-AL0-abp-a-12-0-UI s1 Mus musculus cDNA, 3 end/clone = UI-M-AL0-abp-a-12-0-UI/ | 762.4 | P | 511.3 | P | 541 | P |
| | | clone_end = 3/gb = AI838527/gi = 5472705/ug = Mm.24831/len = 305/STRA = for | | | | | | |
| 3 | 94438_at | Cluster Incl AI852672 UI-M-BH0-aji-b-09-0-UI s1 Mus musculus cDNA, 3 end/clone = UI-M-BH0-aji-b-09-0-UI/clone_end = 3/ | 1177.4 | P | 658.4 | P | 792.9 | P |
| | | gb = AI852672/gi = 5496578/ug = Mm 26550/len = 381/STRA = for | | | | | | |
| 3 | 94952_at | Cluster Incl AI152780 ud91a10 rt Mus musculus cDNA, 5 end/clone = IMAGE-1478202/clone_end = 5/gb = AI152780/ | 1205 | P | 1198.9 | P | 1086.2 | P |
| | | gi = 3681249/ug = Mm.27118/len = 250/STRA = for | | | | | | |
| 3 | 94968_at | Cluster Incl AW048793: UI-M-BH1-amg-c-06-0-UI s1 Mus musculus cDNA, 3 end/clone = UI-M-BH1-amg-c-06-0-UI/ | 1194.9 | P | 1090.1 | P | 1129.4 | P |
| | | clone_end = 3/gb = AW048793/gi = 5909322/ug = Mm 27220/len = 335/STRA = for | | | | | | |
| 3 | 95531_at | Cluster Incl AI854771 UI-M-BH0-aka-c-10-0-UI s1 Mus musculus cDNA, 3 end/clone = UI-M-BH0-aka-c-10-0-UI/ | 329.1 | P | 303.8 | P | 260.9 | P |
| | | clone_end = 3/gb = AI854771/gi = 5498677/ug = Mm.27633/len = 519/STRA = for | | | | | | |
| 3 | 95547_at | Cluster Incl D89821 Mus musculus mRNA for RhoM, complete cds/cds = (84,716)/gb = D89821/gi = 1752824/ug = Mm 27701/ | 354.2 | P | 292 | P | 288 | P |
| | | len = 1166/STRA = for | | | | | | |
| 3 | 95587_at | Cluster Incl AI837204 UI-M-AK0-add-f-07-0-UI.s1 Mus musculus cDNA, 3 end/clone = UI-M-AK0-add-f-07-0-UI/ | 496.6 | P | 482.6 | P | 471 | P |
| | | clone_end = 3/gb = AI837204/gi = 5471417/ug = Mm.27862/len = 287/STRA = for | | | | | | |
| 3 | 95607_at | Cluster Incl X82457 M. musculus es64 mRNA/cds = (176,1516)/gb = X82457/gi = 951299/ug = Mm.27983/len = 2054/ | 453 | P | 504.8 | P | 507.4 | P |
| | | STRA = for | | | | | | |
| 3 | 95625_at | Cluster Incl AI840149 UI-M-AO0-acc-c-09-0-UI.s1 Mus musculus cDNA, 3 end/clone = UI-M-AO0-acc-c-09-UI/ | 421.8 | P | 561.9 | P | 342.2 | P |
| | | clone_end = 3/gb = AI840149/gi = 5474362/ug = Mm 28056/len = 339/STRA = for | | | | | | |
| 3 | 96140_at | Cluster Incl AF039021: Mus musculus pencentriolar material 1 (Pcm1) gene, partial cds/cds = (0,196)/gb = AF039021/ | 205.3 | P | 191.8 | P | 192 | P |
| | | gi = 3170134/ug = Mm.28192/len = 197/STRA = for | | | | | | |
| 3 | 96154_at | Cluster Incl AA600645: vm77b04 r1 Mus musculus cDNA, 5 end/clone = IMAGE-1004239/clone_end = 5/gb = AA600645/ | 635.4 | P | 458.7 | P | 462.3 | P |
| | | gi = 2434270/ug = Mm.28280/len = 378/STRA = for | | | | | | |
| 3 | 96156_at | Cluster Incl AA874329 vx79h02.r1 Mus musculus cDNA, 5 end/clone = IMAGE-1281459/clone_end = 5/gb = AA874329/ | 273.2 | P | 254 | P | 230.9 | P |
| | | gi = 2979018/ug = Mm.28311/len = 206/STRA = for | | | | | | |
| 3 | 96186_at | Cluster Incl AI839288 UI-M-AO0-acb-c-08-0-UI s1 Mus musculus cDNA, 3 end/clone = UI-M-AO0-acb-c-08-0-UI/ | 591.3 | P | 570.3 | P | 587.5 | P |
| | | clone_end = 3/gb = AI839286/gi = 5473499/ug = Mm.28465/len = 258/STRA = for | | | | | | |
| 3 | 96200_at | Cluster Incl AI838344 UI-M-AO0-aby-h-11-0-UI s1 Mus musculus cDNA, 3 end/clone = UI-M-AO0-aby-h-11-0-UI/ | 553.5 | P | 527.3 | P | 500 | P |
| | | clone_end = 3/gb = AI838344/gi = 5472557/ug = Mm.28595/len = 393/STRA = for | | | | | | |
| 3 | 96731_at | Cluster Incl AA615100: vo34f04 r1 Mus musculus cDNA, 5 end/clone = IMAGE-1051807/clone_end = 5/gb = AA615100/ | 945.9 | P | 733.4 | P | 596.7 | P |
| | | gi = 2502328/ug = Mm 28869/len = 145/STRA = for | | | | | | |
| 3 | 96747_at | Cluster Incl AW121294 UI-M-BH2 3-aog-d-09-0-UI s1 Mus musculus cDNA, 3 end/clone = UI-M-BH2 3-aog-d-09-0-UI/ | 482.3 | P | 591.2 | P | 540 | P |
| | | clone_end = 3/gb = AW121294/gi = 6096627/ug = Mm.28954/len = 235/STRA = for | | | | | | |
| 3 | 96761_at | Cluster Incl AF109906: Mus musculus MHC class III region RD gene, partial cds; Bf, C2, G9A, NG22, G9, HSP70, HSP70, | 801.4 | P | 675.1 | P | 616.6 | P |
| | | HSC70t, and smRNP genes, complete cds; G7A gene, partial cds; and unknown genes/cds = (0,2123)/gb = AF 109906/ | | | | | | |
| | | gi = 3986763/ug = Mm.29004/len = 212/STRA = for | | | | | | |
| 3 | 96771_at | Cluster Incl AI1006228 ua88d09 r1 Mus musculus cDNA, 5 end/clone = IMAGE-1364561/clone_end = 5/gb = AI006228/ | 1113.6 | P | 1144 | P | 1156.3 | P |
| | | gi = 3215837/ug = Mm 29023/len = 382/STRA = rev | | | | | | |
| 3 | 96837_at | Cluster Incl AI837492 UI-M-ALO-abr-f-11-0-UI s1 Mus musculus cDNA, 3 end/clone = UI-M-AL0-abr-f-11-0-UI/clone_end = 3/ | 1363.2 | P | 1271.3 | P | 1293.9 | P |
| | | gb = AI837492/gi = 5471705/ug = Mm.29442/len = 181/STRA = for | | | | | | |
| 3 | 97357_at | Cluster Incl AI426400 mm50h04 x1 Mus musculus cDNA, 3 end/clone = IMAGE-524983/clone_end = 3/gb = AI426400/ | 149.9 | P | 162.6 | P | 180 | P |
| | | gi = 4272326/ug = Mm 29635/len = 157/STRA = for | | | | | | |
| 3 | 97929_r_at | Cluster Incl AW124782: UI-M-BH2.1-aqa-h-02-0-UI s1 Mus musculus cDNA, 3 end/clone = UI-M-BH2 1-aqa-h-02-0-UI/ | 989.8 | P | 967.2 | P | 854.5 | P |
| | | clone_end = 3/gb = AW124782/gi = 6100312/ug = Mm 30237/len = 484/STRA = for | | | | | | |
| 3 | 97935_at | Cluster Incl AI842970 UI-M-AO1-aem-g-06-0-UI s1 Mus musculus cDNA, 3 end/clone = UI-M-AO1-aem-g-06-0-UI/ | 530.4 | P | 568.3 | P | 586.2 | P |
| | | clone_end = 3/gb = AI842970/gi = 5477183/ug = Mm 30252/len = 374/STRA = for | | | | | | |
| 3 | 97994_at | Cluster Incl AI019193 ub20e12 r1 Mus musculus cDNA, 5 end/clone = IMAGE-1378318/clone_end = 5/gb = AI019193/ | 398.2 | P | 444.4 | P | 412.2 | P |
| | | gi = 3233529/ug = Mm 31630/len = 476/STRA = for | | | | | | |
| 3 | 98027_at | Cluster Incl Z22923 Procollagen, type IX, alpha 2/cds = (383,2449)/gb = Z22923/gi = 311949/ug = Mm 3277/len = 2956/ | 460.3 | P | 379.2 | P | 348.8 | P |
| | | STRA = for | | | | | | |
| 3 | 98426_at | Cluster Incl AF030001 Mus musculus major histocompatibility locus class III region-butyrophilin-like protein gene, partial | 196.2 | P | 178.3 | P | 132.5 | P |
| | | cds, Notch4, PBX2, RAGE, lysophatidic acid acyl transferase-alpha, palmitoyl-protein thioesterase 2 (PPT2), CREB-RP, | | | | | | |
| | | and tenascin X/STRA = for | | | | | | |
| 3 | 98435_at | Cluster Incl M74495 Mouse adenylosuccinate synthetase mRNA, complete cds/cds = (30,1403)/gb = M74495/gi = 404750/ | 321.9 | P | 322.9 | P | 303.9 | P |
| | | ug = Mm 3440/len = 1736/STRA = for | | | | | | |
| 3 | 98454_at | Cluster Incl Y14771 Mus musculus mRNA for paralernmin/cds = (127,1278)/gb = Y14771/gi = 4456676/ug = Mm 34650/ | 623.6 | P | 561.8 | P | 600.6 | P |
| | | len = 1936/STRA = for | | | | | | |
| 3 | 98460_at | Cluster Incl AJ237917 Mus musculus mRNA for fatso protein/cds = (37,1545)/gb = AJ237917/gi = 6102999/ug = Mm.34778/ | 648.2 | P | 607.8 | P | 595.2 | P |
| | | len = 3554/STRA = for | | | | | | |
| 3 | 98476_at | Cluster Incl L40631 Ankyrin 3, epithelial/cds = (647,3931)/gb = L40631/gi = 710545/ug = Mm 3526/len = 5074/STRA = for | 244.6 | P | 426.9 | P | 330.1 | P |
| 3 | 98989_at | Cluster Incl AF057368 7-dehydrocholesterol reductase/cds = (39,1454)/gb = AF057368/gi = 3283173/ug = Mm 3747/ | 245.5 | P | 242.3 | P | 244.1 | P |
| | | len = 2377/STRA = rev | | | | | | |
| 3 | 99009_at | Cluster Incl Z49204 Nicotinamide nucleotide transhydrogenase/cds = (68,3328)/gb = Z49204/gi = 840318/ug = Mm 3842/ | 482.3 | P | 308.3 | P | 337.3 | P |
| | | len = 3844/STRA = for | | | | | | |
| 3 | 99024_at | Cluster Incl U32395 Mus musculus Max-interacting transcriptional repressor (Mad4) mRNA, complete cds/cds = (0,629)/ | 1158.1 | P | 1073.4 | P | 1243.6 | P |
| | | gb = U32395/gi = 1184158/ug = Mm.38795/len = 630/STRA = for | | | | | | |
| 3 | 99073_at | Cluster Incl Z47766 Cyclin F/cds = (39,2369)/gb = Z47766/gi = 833246/ug = Mm 4008/len = 2815/STRA = for | 353.8 | P | 276.5 | P | 299.7 | P |
| 3 | 99448_at | Cluster Incl X56123 Mouse mRNA for talin/cds = UNKNOWN/gb = X56123/gi = 54257/ug = Mm 4053/len = 8233/STRA = for | 554.1 | P | 760.2 | P | 669.1 | P |
| 3 | 99471_at | Cluster Incl AF158183 AF158183 Mus musculus cDNA/gb = AF158183/gi = 5499038/ug = Mm 41271/len = 1034/STRA = for | 822.1 | P | 841.8 | P | 585.3 | P |
| 3 | 100003_at | Cluster Incl D38216 Mouse RyR1 mRNA for skeletal muscle ryanodine receptor, partial cds/cds = (0,371)/gb = D38216/ | 342.5 | P | 412 | P | 453.5 | P |
| | | gi = 1030707/ug = Mm 4519/len = 486/STRA = for | | | | | | |
| 3 | 100436_at | Cluster Incl M27008 Orosomucoid 1/cds = (32,655)/gb = M27008/gi = 531206/ug = Mm 4777/len = 768/STRA = for | 361.6 | P | 475.5 | P | 470.4 | P |
| 3 | 100440_f_at | Cluster Incl U76758 Ankyrin 1, erythroid/cds = (0,164)/gb = U76758/gi = 1698953/ug = Mm 4789/len = 191/STRA = for | 1066.7 | P | 814.1 | P | 793 | P |
| 3 | 100457_at | Cluster Incl X84037: M musculus mRNA for E-selectin ligand-1/cds = (6,3533)/gb = X84037/gi = 673435/ug = Mm 488/ | 985.5 | P | 918.3 | P | 1157.8 | P |
| | | len = 3887/STRA = for | | | | | | |
| 3 | 100486_at | Cluster Incl U60453 Mus musculus enhancer of zeste homolog 1 (Ezh1) mRNA, complete cds/cds = (108,2351)/ | 472.2 | P | 478.6 | P | 423.5 | P |
| | | gb = U60453/gi = 1575340/ug = Mm.5027/len = 3970/STRA = for | | | | | | |
| 3 | 100509_at | Cluster Incl AW121012 UI-M-BH2.3-aod-h-06-0-UI.s1 Mus musculus cDNA, 3 end/clone = UI-M-BH2.3-aod-h-06-0-UI/ | 309.9 | P | 321.3 | P | 270.7 | P |
| | | clone_end = 3/gb = AW121012/gi = 6096345/ug = Mm 5181/len = 204/STRA = for | | | | | | |
| 3 | 100893_at | Cluster Incl U27455 Serine palmitoyltransferase, long chain base subunit 2$$/cds = (27,1709)/gb = U27455/gi = 2267216/ | 493.6 | P | 621.2 | P | 415.3 | P |
| | | ug = Mm 565/len = 1893/STRA = rev | | | | | | |
| 3 | 100896_at | Cluster Incl AJ006306 Mus musculus mRNA for skeletal muscle calcium channel, gamma-subunit/cds = (131,802) | 416.5 | P | 433.8 | P | 537.8 | P |
| | | gb = AJ006306/gi = 3157401/ug = Mm 57093/len = 803/STRA = for | | | | | | |
| 3 | 101383_at | Cluster Incl AJ131711.Mus musculus mRNA for troponin T/cds = (1,789)/gb = AJ131711/gi = 4468629/ug = Mm 711/len = 907/ | 600.8 | P | 394.8 | P | 354.7 | P |
| | | STRA = for | | | | | | |
| 3 | 101394_at | Cluster Incl AB024922: Mus musculus mRNA for gamma-sarcogiycan, complete cds/cds = (256,1131)/gb = AB024922/ | 293.8 | P | 193 | P | 176.5 | P |
| | | gi = 5771417/ug = Mm 72173/len = 1289/STRA = for | | | | | | |
| 3 | 101451_at | Cluster Incl AF038939 Mus musculus zinc finger protein (Peg3) mRNA, complete cds/cds = (13,4726)/gb = AF038939/ | 2853.2 | P | 17799.8 | P | 2287.8 | P |
| | | gi = 2791677/ug = Mm 7952/len = 5560/STRA = for | | | | | | |
| 3 | 101458_at | Cluster Incl D30743.Mouse mRNA for wee 1 kinase/cds = (292,2232)/gb = D30743/gi = 765088/ug = Mm.8108/len = 2270/ | 49.2 | P | −0.7 | P | 34.6 | P |
| | | STRA = for | | | | | | |
| 3 | 101889_s_at | Cluster Incl U53228 RAR-related orphan receptor alpha/cds = (5,1576)/gb = U53228/gi = 1289325/ug = Mm 8858/len = 1657/ | 1044.3 | P | 613.9 | P | 1125.7 | P |
| | | STRA = for | | | | | | |
| 3 | 101892_f_at | Cluster Incl Z67963: Fused toes/cds = (213,1091)/gb = Z67963/gi = 1067144/ug = Mm 88777/len = 2173/STRA = for | 271.6 | P | 296.9 | P | 408.4 | P |
| 3 | 101897_g_at | Cluster Incl M63697 CD1d2 antigen/cds = (293,1303)/gb = M63697/gi = 192473/ug = Mm 88779/len = 1728/STRA = for | 465.5 | P | 401.7 | P | 348.1 | P |
| 3 | 101943_at | Cluster Incl AA960259 ub61a12 s1 Mus musculus cDNA, 3 end/clone = IMAGE-1382206/clone_end = 3/gb = AA960259/ | 546.9 | P | 556.8 | P | 579 | P |
| | | gi = 3126159/ug = Mm 95636/len = 398/STRA = for | | | | | | |
| 3 | 102223_at | Cluster Incl AF013715 Periplakin/cds = (0,331)/gb = AF013715/gi = 2992536/ug = Mm 10225/len = 776/STRA = for | 2534.5 | P | 2217.5 | P | 2182.9 | P |
| 3 | 102224_at | Cluster Incl AF056187 Mus musculus insulin-like growth factor I receptor mRNA, complete cds/cds = (0,4121)/ | 1307.3 | P | 1195.8 | P | 1252.3 | P |
| | | gb = AF056187/gi = 3025893/ug = Mm 10226/len = 4500/STRA = rev | | | | | | |
| 3 | 102304_f_at | Cluster Incl L28167 Zinc finger protein 61/cds = (927,2594)/gb = L28167/gi = 758660/ug = Mm,12832/len = 2606/STRA = for | 210.2 | P | 222.1 | P | 184.9 | P |
| 3 | 102866_at | Cluster Incl X94441: TEA domain family member 4/cds = (0,1283)/gb = X9441/gi = 1403383/ug = Mm.14744/len = 1284/ | 990.5 | P | 1111.6 | P | 1321.3 | P |
| | | STRA = for | | | | | | |
| 3 | 102867_at | Cluster Incl U51743: TEA domain family member 4/cds = (0,1138)/gb = U51743/gi = 1381555/ug = Mm.14774/len = 1339/ | 210.3 | P | 204.5 | P | 181 | P |
| | | STRA = for | | | | | | |
| 3 | 102890_at | Cluster Incl U00677: Syntrophin, acidic 1/cds = (110,1621)/gb = U00677/gi = 404930/ug = Mm.1541/len = 2109/STRA = for | 769.3 | P | 958.6 | P | 800.4 | P |
| 3 | 102915_at | Cluster Incl Z48043 Coagulation factor II (thrombin) receptor-like 1/cds = (72,1271)/gb = Z48043/gi = 663020/ug = Mm.1614/ | 349.4 | P | 315.6 | P | 318.4 | P |
| | | len = 2713/STRA = for | | | | | | |
| 3 | 103218_at | Cluster Incl J04761 Mouse housekeeping DXS253E (P3) and DXS254E (GdX) genes, 3 end, and complete cds/ | 481.2 | P | 466.2 | P | 424.3 | P |
| | | cds = (0,549)/gb = J04761/gi = 202256/ug = Mm.19931/len = 550/STRA = for | | | | | | |
| 3 | 103466_at | Cluster Incl AA790056 vt78h04 r1 Mus musculus cDNA, 5 end/clone = IMAGE-1177303/clone_end = 5/gb = AA790058/ | 329.4 | P | 267.3 | P | 257.7 | P |
| | | gi = 2850176/ug = Mm.21783/len = 490/STRA = for | | | | | | |
| 3 | 103534_at | Cluster Incl V00722 Mouse gene for beta-1-giobin/cds = (53,496)/gb = V00722/gi = 50155/ug = Mm.2329/len = 626/STRA = for | 3796.6 | P | 3660 | P | 5631.2 | P |
| 3 | 103720_at | Cluster Incl AI449034 ms60f04.x1 Mus musculus cDNA, 3 end/clone = IMAGE-615967/clone_end = 3/gb = AI449034/ | 912.1 | P | 585.6 | P | 671.4 | P |
| | | gi = 4287173/ug = Mm.24194/len = 563/STRA = rev | | | | | | |
| 3 | 103721_at | Cluster Incl AA592182: vo15b01 r1 Mus musculus cDNA, 5 end/clone = IMAGE-1049929/clone_end = 5/gb = AA592182/ | 323.2 | P | 209.8 | P | 282.8 | P |
| | | gi = 2405845/ug = Mm.24198/len = 494/STRA = for | | | | | | |
| 3 | 103770_at | Cluster Incl AA794189 vu67d02 r1 Mus musculus cDNA, 5 end/clone = IMAGE-1196451/clone_end = 5/gb = AA794189/ | 287.3 | P | 534.6 | P | 581.8 | P |
| | | gi = 2857144/ug = Mm.24776/len = 500/STRA = for | | | | | | |
| 3 | 103958_g_at | Cluster Incl X57349 M. musculus mRNA for transferrin receptor/cds = (0,2291)/gb = X57349/gi = 54914/ug = Mm 25560/ | 2787.9 | P | 2426.8 | P | 2007.1 | P |
| | | len = 2292/STRA = for | | | | | | |
| 3 | 103964_at | Cluster Incl U85259 Estrogen receptor related 1/cds = (166,1428)/gb = U85259/gi = 1916860/ug = Mm 2563/len = 2203/ | 637.1 | P | 680.2 | P | 620.2 | P |
| | | STRA = for | | | | | | |
| 3 | 104188_at | Cluster Incl AI853703 UI-M-BH0-ajq-c-08-0-UI s1 Mus musculus cDNA, 3 end/clone = UI-M-BH0-ajq-c-08-0-UI/ | 400.4 | P | 395 | P | 288.2 | P |
| | | clone_end = 3/gb = AI853703/gi = 5497574/ug = Mm 27449/len = 485/STRA = for | | | | | | |
| 3 | 104196_at | Cluster Incl AA711908 vu59e03 r1 Mus musculus cDNA, 5 end/clone = IMAGE-1195708/clone_end = 5/gb = AA711908/ | 306.2 | P | 314.9 | P | 229.9 | P |
| | | gi = 2721826/ug = Mm 27512/len = 474/STRA = for | | | | | | |
| 3 | 104199_at | Cluster Incl AI646237 vv88b02.x1 Mus musculus cDNA, 3 end/clone = IMAGE-1229451/clone_end = 3/gb = AI646237/ | 701 | P | 618.5 | P | 511.4 | P |
| | | gi = 4724712/ug = Mm.27521/len = 347/STRA = for | | | | | | |
| 3 | 104218_s_at | Cluster Incl AI507524 vl53f03 x1 Mus musculus cDNA, 3 end/clone = IMAGE-975965/clone_end = 3/gb = AI507524/ | 279.9 | P | 237 | P | 301.8 | P |
| | | gi = 4405375/ug = Mm.27913/len = 433/STRA = for | | | | | | |
| 3 | 104250_at | Cluster Incl AW121353: UI-M-BH2 2-aom-c-12-0-UI s1 Mus musculus cDNA, 3 end/clone = UI-M-BH2.2-aom-c-12-0-UI/ | 438.5 | P | 355.6 | P | 532.6 | P |
| | | clone_end = 3/gb = AW121353/gi = 6096686/ug = Mm.28355/len = 562/STRA = for | | | | | | |
| 3 | 104264_at | Cluster Incl AW122230.UI-M-BH2 2-aov-e-05-0-UI.s1 Mus musculus cDNA, 3 end/clone = UI-M-BH2.2aov-e-05-0-UI/ | 528.2 | P | 383.4 | P | 392.4 | P |
| | | clone_end = 3/gb = AW122230/gi = 6097693/ug = Mm.28458/len = 266/STRA = for | | | | | | |
| 3 | 104436_at | Cluster Incl AJ250429 Mus musculus mRNA for IL-1L1 protein/cds = (112,579)/gb = AJ250429/gi = 6165412/ug = Mm.29261/ | 1054.8 | P | 968.3 | P | 989.9 | P |
| | | len = 1284/STRA = rev | | | | | | |
| 3 | 104453_at | Cluster Incl AW046336.UI-M-BH1-ale-f-04-0-UI s1 Mus musculus cDNA, 3 end/clone = UI-M-BH1-ale-f-04-0-UI/ | 636 | P | 710.5 | P | 651.4 | P |
| | | clone_end = 3/gb = AW046336/gi = 5906865/ug = Mm.2947/len = 298/STRA = rev | | | | | | |
| 3 | 104471_at | Cluster Incl AF006603 Mus musculus histone deacetylase mHDA2 mRNA, complete cds/cds = (0,3287)/gb = AF006603/ | 459.9 | P | 532.9 | P | 539.9 | P |
| | | gi = 4165860/ug = Mm.29854/len = 3651/STRA = for | | | | | | |
| 3 | 104502_f_at | Cluster Incl AI414025 ma02g05.x1 Mus musculus cDNA, 3 end/clone = IMAGE-303416/clone_end = 3/gb = AI414025/ | 224.6 | P | 143.4 | P | 264.1 | P |
| | | gi = 4257529/ug = Mm.30608/len = 346/STRA = for | | | | | | |
| 3 | 104638_at | Cluster Incl U31510 ADP-ribosyltransferase 1/cds = (279,1256)/gb = U31510/gi = 1209033/ug = Mm.31099/len = 1640/ | 178 | P | 349.6 | P | 367.8 | P |
| | | STRA = for | | | | | | |
| 3 | 104673_at | Cluster Incl X65138 Eph receptor A4/cds = (57,3017)/gb = X65138/gi = 54083/ug = Mm.3249/len = 4242/STRA = for | 287.7 | P | 244.8 | P | 202.8 | P |
| 3 | 104697_at | Cluster Incl AW121127.UI-M-BH2.3-aoh-d-04-0-UI s1 Mus musculus cDNA, 3 end/clone = UI-M-BH2.3-aoh-d-04-0-UI/ | 297.1 | P | 258 | P | 279.4 | P |
| | | clone_end = 3/gb = AW121127/gi = 6096460/ug = Mm.33722/len = 402 = STRA = for | | | | | | |
| 3 | 92259_at | Cluster Incl AI467526: ve34d12.x1 Mus musculus cDNA, 3 end/clone = IMAGE-820055/clone_end = 3/gb = AI467526/ | 1149.2 | P | 1200.1 | P | 1015.3 | P |
| | | gi = 4320863/ug = Mm.35645/len = 483/STRA = rev | | | | | | |
| 3 | 92265_f_at | Cluster Incl AF042139 Mus musculus 60 kDa ribonucleoprotein Ro gene, partial cds/cds = (0,1375)/gb = AF042139/ | 913 | P | 1108.5 | P | 881.4 | P |
| | | gi = 3098483/ug = Mm 35793/len = 1376/STRA = for | | | | | | |
| 3 | 92371_at | Cluster Incl AI505355: vq02d11.x1 Mus musculus cDNA, 3 end/clone = IMAGE-1093077/clone_end = 3/gb = AI505355/ | 440.2 | P | 504.2 | P | 412.1 | P |
| | | gi = 4403206/ug = Mm.39968/len = 421/STRA = for | | | | | | |
| 3 | 92407_at | Cluster Incl AJ012072.Mus musculus mRNA for myomesin/skelemin/cds = (158,5158)/gb = AJ012072/gi = 5419921/ | 339.9 | P | 224.1 | P | 433.6 | P |
| | | ug = Mm.4103/len = 5645/STRA = for | | | | | | |
| 3 | 92466_at | Cluster Incl U85714 Mus musculus phospholipase C-beta-1b mRNA, complete cds/cds = (3,3524)/gb = U85714/ | 1227.2 | P | 1042.6 | P | 893.7 | P |
| | | gi = 4099296/ug = Mm.42083/len = 3853/STRA = for | | | | | | |
| 3 | 92933_at | Cluster Incl Z18537 POU domain, class 2, transcription factor 3/cds = (75,1286)/gb = Z18537/gi = 53514/ug = Mm.4904/ | 871.7 | P | 934.8 | P | 780.6 | P |
| | | len = 1267/STRA = for | | | | | | |
| 3 | 92958_at | Cluster Incl AI849135: UI-M-AJ1-agy-b-09-0-UI.s1 Mus musculus cDNA, 3 end/clone = UI-M-AJ1-agy-b-09-0-UI/ | 685.3 | P | 515 | P | 659.5 | P |
| | | clone_end = 3/gb = AI849135/gi = 5493041/ug = Mm.4948/len = 384 = STRA = for | | | | | | |
| 3 | 93193_at | Cluster Incl X15643.Adrenergic receptor, beta 2/cds = (0,1256)/gb = X15643/gi = 50100/ug = Mm.5598/len = 1257/STRA = for | 381.1 | P | 314.6 | P | 274 | P |
| 3 | 93235_at | Cluster Incl AI020029 ub24c02 r1 Mus musculus cDNA, 5 end/clone = IMAGE-1378658/clone_end = 5/gb = AI020029/ | 390.6 | P | 399.9 | P | 399 | P |
| | | gi = 3234365/ug = Mm.5833/len = 468/STRA = for | | | | | | |
| 3 | 93383_at | Cluster Incl U32107 Procollagen, type VII, alpha 1/cds = (0,8834)/gb = U32107/gi = 2326167/ug = Mm.6200/len = 9235/ | 1425.8 | P | 1084.6 | P | 956.6 | P |
| | | STRA = for | | | | | | |
| 3 | 93451_at | Cluster Incl AW047919 UI-M-BH1-aln-c-05-0-UI.s1 Mus musculus cDNA, 3 end/clone = UI = M-BH1-aln-c-05-0-UI/ | 1509.7 | P | 1312 | P | 882.4 | P |
| | | clone_end = 3/gb = AW047919/gi = 5908448/ug = Mm.6772/len = 413/STRA = for | | | | | | |
| 3 | 93632_g_at | Cluster Incl X95761: M. musculus mRNA for new-Rhobin/cds = UNKNOWN/gb = X95761/gi = 1771307/ug = Mm.7634/ | 395.5 | P | 392.8 | P | 341.7 | P |
| | | len = 2031/STRA = rev | | | | | | |
| 3 | 93681_at | Cluster Incl AW123618: UI-M-BH2.1-api-d-09-0-UI.s1 Mus musculus cDNA, 3 end/clone = UI-M-BH2.1-api-d-09-0-UI/ | 678.4 | P | 553.8 | P | 528.5 | P |
| | | clone_end = 3/gb = AW123618/gi = 6099148/ug = Mm 82649/len = 416/STRA = for | | | | | | |
| 3 | 93903_at | Cluster Incl M84120 Mouse activin receptor (ActR IIB) mRNA, complete cds/cds = (43,1653)/gb = M84120/gi = 191668/ | 452.1 | P | 347.8 | P | 474.7 | P |
| | | ug = Mm.8940/len = 1708/STRA = for | | | | | | |
| 3 | 93907_f_at | Cluster Incl M17551 Mus musculus clone MIA14 full-length intracisternal A-particle gag protein gene, complete cds, and pol | 4219.9 | P | 3547.7 | P | 3993.8 | P |
| | | pseudogene, complete sequence/cds = (0,2483)/gb = M17551/gi = 193325/ug = Mm 89480/len = 2484/STRA = for | | | | | | |
| 3 | 93908_f_at | Cluster Incl X16670 Mouse RNA for type IIB intracisternal A-particle (IAP) element encoding integrase, clone 106/ | 778.1 | P | 732.6 | P | 521.2 | P |
| | | cds = (498,1883)/gb = X16670/gi = 51488/ug = Mm 89480/len = 1931/STRA = for | | | | | | |
| 3 | 93909_f_at | Cluster Incl X04120 Intracisternal A particles,Thbd linked/cds = (0,2444)/gb = X04120/gi = 51502/ug = Mm 89480/len = 2445/ | 1320.6 | P | 1284.3 | P | 1170 | P |
| | | STRA = for | | | | | | |
| 3 | 94106_at | Cluster Incl AI853048 UI-M-BH0-ajn-f-11-0-UI s1 Mus musculus cDNA, 3 end/clone = UI-M-BH0-ajn-f-11-0-UI/ | 569.2 | P | 691.1 | P | 555.3 | P |
| | | clone_end = 3/gb = AI853048/gi = 5496954/ug = Mm 1024/len = 348/STRA = for | | | | | | |
| 3 | 94802_at | Cluster Incl AI846678 UI-M-AN1-afi-d-11-0-UI.s1 Mus musculus cDNA, 3 end/clone = UI-M-AN1-afi-d-11-0-UI/ | 249.6 | P | 222.8 | P | 271.5 | P |
| | | clone_end = 3/gb = AI846678/gi = 5490584/ug = Mm 1835/len = 142/STRA = for | | | | | | |
| 3 | 95322_g_at | Cluster Incl Y09588 Mus musculus mRNA for serotonin 4 receptor (m5-HT4(e)), splice vanant/cds = (2,1117)/gb = Y09588/ | 1082.5 | P | 1026.9 | P | 841.4 | P |
| | | gi = 3647302/ug = Mm 20440/len = 1266/STRA = for | | | | | | |
| 3 | 95366_at | Cluster Incl L35599 Mus musculus Y-box binding protein mRNA, 3 end/cds = (0,825)/gb = L35599/gi = 532212/ | 260.8 | P | 274.9 | P | 225.1 | P |
| | | ug = Mm.22482/len = 2437/STRA = for | | | | | | |
| 3 | 96578_r_at | Cluster Incl AI853019 UI-M-BHo-ajn-d-04-0-UI s1 Mus musculus cDNA, 3 end/clone = UI-M-BH0-ajn-d-04-0-UI/ | 442.9 | P | 521.2 | P | 387.6 | P |
| | | clone_end = 3/gb = AI853019/gi = 5496925/ug = Mm 3021/len = 250/STRA for | | | | | | |
| 3 | 97116_at | Cluster Incl AW123210 UI-M-BH2.1-apg-d-05-0-UI s1 Mus musculus cDNA, 3 end/clone = UI-M-BH2 1-apg-d-05-0-UI/ | 421.3 | P | 332 | P | 267.4 | P |
| | | clone_end = 3/gb = AW123210/gi = 6098780/ug = Mm 3369/len = 468/STRA for | | | | | | |
| 3 | 97138_at | Cluster Incl C77335 C773355 Mus musculus cDNA, 3 end/clone = J0029D01/clone_end = 3/gb = C77335/gi = 2517665/ | 342.9 | P | 233.2 | P | 232.3 | P |
| | | ug = Mm 34933/len = 582/STRA = rev | | | | | | |
| 3 | 97160_at | Cluster Incl X04017: Mouse mRNA for cysteine-rich glycoprotein SPARC/cds = (89,997)/gb = X04017/gi = 54168/ | 4145.9 | P | 3274 | P | 3958.7 | P |
| | | ug = Mm 35439/len = 2079/STRA = for | | | | | | |
| 3 | 97181_f_at | Cluster Incl M10062 Mouse IgE-binding factor mRNA, complete cds/cds = UNKNOWN/gb = M10062/gi = 192166/ | 3942.9 | P | 4035.7 | P | 4101.1 | P |
| | | ug = Mm 35835/len = 3305/STRA = for | | | | | | |
| 3 | 97759_at | Cluster Incl U09383 Mus musculus calcium-activated potassium channel mRNA, complete cds/cds = (820,4374)/ | 155.9 | P | 149.8 | P | 140.9 | P |
| | | gb = U09383/gi = 487796/ug = Mm 4123/len = 5001/STRA = for | | | | | | |
| 3 | 98278_at | Cluster Incl AB019280 Mus musculus mRNA for sprouty-4, complete cds/cds = (215,1117)/gb = AB019280/gi = 4850325/ | 820.6 | P | 908.8 | P | 714.8 | P |
| | | ug = Mm 42038/len = 1131/STRA = for | | | | | | |
| 3 | 99405_at | Cluster Incl U30241 Mus musculus anti-DNA antibody Ig kappa chain mRNA, v-j region, hybndoma 84.15, partial cds/ | 234.5 | P | 254.2 | P | 203.3 | P |
| | | cds = (0,298)/gb = U30241/gi = 896180/ug = Mm 57033/len = 299/STRA for | | | | | | |
| 3 | 99823_r_at | Cluster Incl C79676 C79676 Mus musculus cDNA, 3 end/clone = J0070C12/clone_end = 3/gb = C79676/gi = 2520006/ | 482.2 | P | 477.9 | P | 367.3 | P |
| | | ug = Mm 5866/len = 532/STRA = rev | | | | | | |
| 3 | 99875_at | Cluster Incl Z32675 Hairless/cds = (377,3925)/gb = Z32675/gi 531706/ug = Mm 7598/len = 4017/STRA = for | 379.3 | P | 780.1 | P | 627.6 | P |
| 3 | 99896_at | Cluster Incl U08354 Melanocortin 5 receptor/cds = (28,1146)/gb = U08354/gi = 522165/ug = Mm 8003/len 1180/STRA = for | 374 | P | 329.9 | P | 362.4 | P |
| 3 | 100293_at | Cluster Incl AA268823 vb01d09 r1 Mus musculus cDNA, 5 end/clone = IMAGE-747665/clone_end = 5/gb = AA268823/ | 222.1 | P | 276.3 | P | 239.3 | P |
| | | gi = 1905739/ug = Mm 86378/len = 435/STRA = for | | | | | | |
| 3 | 96963_s_at | Cluster Incl L14553 Mus musculus Ig light chain V-region CC49 rearranged gene, exons 1 and 2/cds = (0,399)/gb = L14553/ | 208.7 | P | 177 | P | 155.9 | P |
| | | gi = 293639/ug = Mm 88797/len = 400/STRA = for | | | | | | |
| 3 | 97058_f_at | Cluster Incl AW208630: uo62a10.x1 Mus musculus cDNA, 3 end/clone = IMAGE-2647098/clone_end = 3/gb = AW208630/ | 670.4 | P | 567.7 | P | 599.1 | P |
| | | gi = 6514570/ug = Mm 89273/len = 197/STRA = rev | | | | | | |
| 3 | 97659_f_at | Cluster Incl X04725.Insulin I/cds = (0,326)/gb = X04725/gi = 52712/ug = Mm 89940/len = 327/STRA = for | 896.2 | P | 894.4 | P | 1104.1 | P |
| 3 | 98245_at | Cluster Incl C77789 C77789 Mus musculus cDNA, 3 end/clone = J0037F03/clone_end = 3/gb = C77789/gi = 2518119/ | 401.5 | P | 412.4 | P | 337.4 | P |
| | | ug = Mm 91361/len = 623/STRA = rev | | | | | | |
| 3 | 100225_f_at | Cluster Incl AA09481 EST01210 Mus musculus cDNA, 3 end/clone = C0007B09/clone_end = 3/gb = AA409481/ | 388 | P | 280.8 | P | 316 | P |
| | | gi = 2066125/ug = Mm.98618/len = 254/STRA = rev | | | | | | |
| 3 | 101152_at | Cluster Incl Z18278 5-hydroxytryptamine (serotonin) receptor 5A/cds = (502,1575)/gb = Z18278/gi = 49758/ug = Mm 4835/ | 510.3 | P | 446.1 | P | 312.5 | P |
| | | len = 1680/STRA = for | | | | | | |
| 3 | 101186_at | Cluster Incl X65588 M musculus mp41 mRNA/cds = (15,416)/gb = X65588/gi = 1524150/ug = Mm 5118/len = 2857/STRA = for | 263.1 | P | 252.3 | P | 309.7 | P |
| 3 | 101217_at | cluster Incl D18885 MUSGS01047 Mus musculus cDNA, 3 end/clone = mc0128/clone_end = 3/gb = D18865/gi = 1089498/ | 267.2 | P | 333 | P | 352.4 | P |
| | | ug = Mm.5344/len = 314/STRA = rev | | | | | | |
| 3 | 92572_at | Cluster Incl AI509617 vx14h07.y1 Mus musculus cDNA, 5 end/clone = IMAGE-1264477/clone_end = 5/gb = AI509617/ | 150.8 | P | 174.7 | P | 141.3 | P |
| | | gi = 4408522/ug = Mm 10331/len = 448/STRA = rev | | | | | | |
| 3 | 92887_at | Cluster Incl AF004106 Mus musculus unknown mRNA, complete cds/cds = (234,968)/gb = AF004106/gi = 2183320/ | 1727.3 | P | 1424.8 | A | 1538.8 | P |
| | | ug = Mm 1457/len = 1270/STRA = for | | | | | | |
| 3 | 93020_at | Cluster Incl AF051347 Mus musculus REX-3 mRNA, complete cds/cds = (228,578)/gb = AF051347/gi = 3510642/ | 121.4 | P | 90.3 | P | 129.6 | P |
| | | ug = Mm.14768/len = 838/STRA = for | | | | | | |
| 3 | 93821_at | Cluster Incl AW046101.UI-M-BH1-alf-b-06-0-UI s1 Mus musculus cDNA, 3 end/clone = UI-M-BH1-alf-b-06-0-UI/ | 297.5 | P | 219.8 | P | 266.5 | P |
| | | clone_end = 3/gb = AW046101/gi = 5906630/ug = Mm 21514/len = 261/STRA = rev | | | | | | |
| 3 | 94877_at | Cluster Incl AI848446 UI-M-AH1-ags-f-03-0-UI s1 Mus musculus cDNA, 3 end/clone = UI-M-AH1-ags-f-03-0-UI/ | 89.5 | P | 95.8 | P | 86.8 | A |
| | | clone_end = 3/gb = AI848446/gi = 5492352/ug = Mm.23839/len = 514/STRA = for | | | | | | |
| 3 | 94472_at | Cluster Incl AW123216 UI-M-BH2.1-apg-d-12-0-UI s1 Mus musculus cDNA, 3 end/clone = UI-M-BH2 1-apg-d-12-0-UI/ | 252.6 | P | 253.7 | P | 193 | P |
| | | clone_end = 3/gb = AW123216/gi = 6098786/ug = Mm 28852/len = 287/STRA = for | | | | | | |
| 3 | 97482_at | Cluster Incl AA832565: vw43b11.r1 Mus musculus cDNA, 5 end/clone = IMAGE-1246557/clone_end = 5/gb = AA832565/ | 412.5 | P | 439.9 | A | 401.5 | P |
| | | gi = 2906293/ug = Mm.30762/len = 478/STRA = for | | | | | | |
| 3 | 97536_at | Cluster Incl AW046650: UI-M-BH1-ald-e-10-0-UI s1 Mus musculus cDNA, 3 end/clone = UI-M-BH1-aid-e-10-0-UI/ | 403.1 | P | 491.2 | P | 409.6 | P |
| | | clone_end = 3/gb = AW046650/gi = 5907179/ug = Mm3308/len = 336/STRA = for | | | | | | |
| 3 | 100997_at | Cluster Incl L22550: Desmin/cds = UNKNOWN/gb = gb 32 L22550/gi = 348971/ug = Mm 6712/len = 1497/STRA = for | 870.3 | P | 1081.3 | P | 840.7 | P |
| 3 | 103616_at | Cluster Incl AI181132.ub95c0.r1 Mus musculus cDNA, 5 end/clone = IMAGE-1396224/clone_end = 5/gb = AI181132/ | 453.7 | P | 255.9 | P | 475.7 | P |
| | | gi = 3731770/ug = Mm20240/len = 499/STRA = for | | | | | | |
| 3 | 104515_at | Cluster Incl AI286818 uc55b02 r1 Mus musculas cDNA, 5 end/clone = IMAGE-1429515/clone_= 5/gb = AI286818/ | 110.4 | P | 167.8 | P | 136.8 | P |
| | | gi = 3926571/ug = Mm.22765/len = 417/STRA = for | | | | | | |
| 3 | 104529_at | Cluster Incl U21960: Calcium modulating ligand/cds = (34,918)/gb = U21960/gi = 726183/ug = Mm 2313/len = 1331/STRA = for | 205 | P | 135.6 | A | 73.3 | P |
| 3 | 98461_at | Cluster Incl AI987835 um07f07.x1 Mus musculus cDNA, 3 end/clone = IMAGE-2182405/clone _end = 3/gb = AI987935/ | 312.8 | P | 305.7 | P | 249.4 | P |
| | | gi = 5819629/ug = Mm 34789/len = 623/STRA = for | | | | | | |
| 3 | 100429_at | Cluster Incl U89155 untitled/cds = (0,149)/gb = U89155/gi = 2707592/ug = Mm 4730/len = 150/STRA = for | 172.1 | P | 156.7 | P | 149.4 | P |
| 3 | 100935_at | Cluster Incl U43548 Mus musculus trasncription factor like protein 4 TCFL4 mRNA, partial cds/cds(0,892)/gb = U43548/ | 536.6 | P | 601.8 | P | 578 | A |
| | | gi = 1927189/ug = Mm.628/len = 1963/STRA = for | | | | | | |
| 3 | 100962_at | Cluster Incl U47543 Ngfi-A binding protein 2/cds = (220,1797)/gb = U47543/gi = 197730/ug = Mm 6549/len = 2539 | 143.9 | P | 166.8 | A | 187.6 | P |
| | | STRA = for | | | | | | |
| 3 | 101359_at | Cluster Incl U43541 Lamin,beta 2/cds = (0,5399)/gb = U43541/gi = 1244719/ug = Mm 68867/len = 5400/STRA = for | 186 | P | 165.7 | P | 135.9 | A |
| 3 | 101861_at | Cluster lncl AF031919 Sarcoglycan, epsilon/cds = (44,1285)/gb = AF031919/gi = 3057139/ug = Mm.8739/len = 1548 | 343.6 | P | 515.4 | A | 371.1 | P |
| 3 | 101913_at | Cluster Incl AW214298 uo39g06.x1 Mus musculus cDNA, 3 end/clone = IMAGE-2644954/clone_end = 3/gb = AW214298/ | 248.6 | P | 271.2 | P | 262.2 | P |
| | | gi = 6520787/ug = Mm.90654/len = 172/STRA = for | | | | | | |
| 3 | 102878_at | Cluster Incl U12135 RAD52 homolog, (S cerevisiae/cds = (114,1373)/gb = U12135/gi = 602158/ug = Mm 149/len = 1664/ | 381.8 | P | 422.6 | P | 363.2 | P |
| | | STRA = for | | | | | | |
| 3 | 103547_at | Cluster Incl AI837116 UI-M-AK0-adc-e-09-0-UI a1 Mus musculus cDNA, 3 end/clone = UI-M-AK0-adc-e-09-0-UI/ | 294.2 | P | 326.9 | P | 278.7 | P |
| | | clone_end = 3/gb = AI837116/gi = 5471329/ug = Mm 23723/len = 323/STRA = rev | | | | | | |
| 3 | 103927_at | Cluster Incl AW212672 uo10c08 x1 Mus musculus cDNA, 3 end/clone = IMAGE-2599118/clone_end = 3/gb = AW212672/ | 384 | P | 185.7 | P | 280.4 | P |
| | | gi = 6518785/ug = Mm 25086/len = 154/STRA = for | | | | | | |
| 3 | 104164_at | Cluster Incl AI181257 ub94b03 r1 Mus musculus cDNA, 5 end/clone = IMAGE-1396109/clone_end = 5/gb = AI181257/ | 188.2 | P | 243.8 | P | 66.8 | P |
| | | gi = 3731895/ug = Mm.27139/len = 510/STRA = for | | | | | | |
| 3 | 104252_at | Cluster Incl AW123823 UI-M-BH2 1-apm-g-07-0-UI s1 Mus musculus cDNA, 3 end/clone = UI-M-BH2 1-apm-g-07-0-UI/ | 166.5 | P | 165.1 | A | 198.4 | P |
| | | clone_end = 3/gb = AW123823/gi = 6099353/ug = Mm 28362/len = 386/STRA = for | | | | | | |
| 3 | 104265_at | Cluster Incl X70842 Kinase insert domain protein receptor/cds = (285,4388)/gb = X70842/gi = 57923/ug = Mm.285/len = 5470/ | 488.3 | P | 473.9 | P | 322.5 | P |
| | | STRA = for | | | | | | |
| 3 | 92269_r_at | Cluster Incl AI836610 UI-M-AP0-abf-g-08-0-UI.s1 Mus musculus cDNA, 3 end/clone = UI-M-AP0-abf-g-08-0-UI/ | 114.7 | P | 81.6 | P | 51.2 | P |
| | | clone_end = 3/gb = AI836610/gi = 5470823/ug = Mm.3590/len = 130/STRA = for | | | | | | |
| 3 | 92518_at | Cluster Incl Y09535 M musculus mRNA for neogenin protein/cds = (180,4661)/gb = Y09535/gi = 1881476/ug = Mm 42249/ | 512.7 | P | 494.7 | P | 443.5 | P |
| | | len = 5414/STRA = for | | | | | | |
| 3 | 92691_at | Cluster Incl AW048155 UI-M-BH1-alq-h-08-0-UI s1 Mus musculus cDNA, 3 end/clone = UI-M-BH1-alq-h-08-0-UI/ | 699 | A | 806 | P | 596.1 | P |
| | | clone_end = 3/gb = AW048155/gi = 5908684/ug = Mm 45608/len = 425/STRA = for | | | | | | |
| 3 | 92942_at | Cluster Incl AA138192: mr05c12 r1 Mus musculus cDNA, 5 end/clone = IMAGE-596566/clone_end = 5/gb = AA138192/ | 833.2 | P | 507.8 | P | 495.2 | P |
| | | gi = 1700519/ug = Mm 492/len = 468/STRA = rev | | | | | | |
| 3 | 93191_at | Cluster Incl AW061328 UI-M-BH1-anw-f-01-0-UI s1 Mus musculus cDNA, 3 end/clone = UI-M-BH1-anw-f-01-0-UI/ | 240.1 | P | 185.2 | P | 151.6 | P |
| | | clone_end = 3/gb = AW061328/gi = 6009079/ug = Mm 5572/len = 461/STRA = rev | | | | | | |
| 3 | 93392_at | Cluster Incl AB010742: Uncoupling protein 3, mitochondrial/cds = (197,1123)/gb = AB010742/gi = 3062840/ug = Mm.6254/ | 306.7 | P | 288.8 | P | 264.2 | P |
| | | len = 2460/STRA = rev | | | | | | |
| 3 | 93690_at | Cluster Incl AF022072 Growth factor receptor bound protein 10/cds = (308,2098)/gb = AF022072/gi = 2465448/ug = Mm 8432/ | 571.5 | P | 493.3 | P | 434 | P |
| | | len = 2176/STRA = for | | | | | | |
| 3 | 94174_at | Cluster Incl AF006071 Mus musculus alpha-catenin related protein (ACRP) mRNA, complete cds/cds = (84,2279)/ | 90.2 | P | 75.3 | P | 76.1 | P |
| | | gb = AF006071/gi = 3309106/ug = Mm.12878/len = 2292/STRA = for | | | | | | |
| 3 | 94790_at | Cluster Incl AA681807: vu65f09 r1 Mus musculus cDNA, 5 end/clone = IMAGE-1196297/clone_end = 5/gb = AA681807/ | 293.2 | P | 168.7 | P | 175.4 | P |
| | | gi = 2663947/ug = Mm 17221/len = 318/STRA = rev | | | | | | |
| 3 | 96541_at | Cluster Incl AI838636 UI-M-AO0-aca-a-10-0-UI.s1 Mus musculus cDNA, 3 end/clone = UI-M-AO0-aca-a-10-0-UI/ | 174.1 | P | 155.1 | P | 144.6 | P |
| | | clone_end = 3/gb = AI838636/gi = 5472849/ug = Mm 27795/len = 369/STRA = rev | | | | | | |
| 3 | 98869_g_at | Cluster Incl L31532 B-cell leukemia/lymphoma 2/cds = (1197,1796)/gb = L31532/gi = 468337/ug = Mm.5155/len = 1897/ | 409.9 | P | 300 | P | 317 | P |
| | | STRA = for | | | | | | |
| 3 | 99813_g_at | Cluster Incl X92523 Calpain 3/cds = (0,2465)/gb = X92523/gi = 1246778/ug = Mm 57235/len = 2466/STRA = for | 84.3 | P | 53.5 | P | 41.7 | P |
| 3 | 99825_at | Cluster Incl AA881307: vx11d09.r1 Mus musculus cDNA, 5 end/clone = IMAGE-1264145/clone_end = 5/gb = AA881307/ | 365.2 | P | 359.9 | P | 281 | P |
| | | gi = 2990617/ug = Mm 5980/len = 511/STRA = rev | | | | | | |
| 3 | 99842_at | Cluster Incl AB000636: Procollagen, type XIX, alpha 1/cds = (98,3508)/gb = AB000636/gi = 2627208/ug = Mm 6252/len = 4205/ | 168 | P | 130.7 | A | 134.6 | P |
| | | STRA = for | | | | | | |
| 3 | 100366_at | Cluster Incl AB019600: Mus musculus mRNA for caspase9, complete cds/cds = (0,1364)/gb = AB019600/gi = 6440941/ | 349.6 | P | 292 | P | 291.5 | P |
| | | ug = Mm.88829/len = 1365/STRA = for | | | | | | |
| 3 | 101716_at | Cluster Incl AF017280 Mus musculus ribonuclease 5 precursor (mR-5) gene, complete cds/cds = (0,467)/gb = AF017260/ | 84.7 | P | 76.7 | P | 61.1 | M |
| | | gi = 2407241/ug = Mm 57025/len = 468/STRA = for | | | | | | |
| 3 | 101830_at | Cluster Incl X83934: Ryanodine receptor 3/ods = (0,866)/gb = X83934/gi = 639816/ug = Mm 57201/len = 867/STRA = for | 248.6 | P | 248.6 | P | 168.6 | A |
| 3 | 101633_at | Cluster Incl X16678: Immunoglobulin kappa chain variable 20 (V20 family)/cds = (0,383)/gb = X16678/gi = 55388/ | 668.4 | P | 843.2 | A | 664.4 | P |
| | | ug = Mm 6294/len = 384/STRA = for | | | | | | |
| 3 | 99187_f_at | Cluster Incl AI835662: UI-M-AJ0-aau-e-03-0-UI s1 Mus musculus cDNA, 3 end/clone = UI-M-AJ0-aau-e-03-0-UI/ | 476.2 | P | 367.5 | A | 461.6 | P |
| | | clone_end = 3/gb = AI835662/gi = 5469875/ug = Mm 41890/len = 268/STRA = for | | | | | | |
| 3 | 103578 _at | Cluster Incl AJ006972: Mus musculus mRNA for TOM1 protein/cds = (67,1545)/gb = AJ006972/gi = 3319987/ug = Mm.1967/ | 330.7 | A | 304.3 | P | 302 | P |
| | | len = 2253/STRA = for | | | | | | |
| 3 | 104591_g_at | Cluster Incl 13171: Myocyte enhancer factor 2C/cds = UNKNOWN/gb = L13171/gi = 293728/ug = Mm.24001/len = 1656/ | 160.4 | P | 91 | P | 98.2 | P |
| | | STRA = for | | | | | | |
| 3 | 97944_f_at | Cluster Incl AF099808: Mus musculus T cell receptor alpha chain variable region (Tcra-V11 1d) gene, allele S1, partial cds/ | 470.8 | P | 351.8 | P | 322.5 | P |
| | | cds = (0,233)/gb = AF099808/gi = 4323544/ug = Mm 30295/len = 234/STRA = for | | | | | | |
| 3 | 101893_r_at | Cluster Incl z67963 Fussed toes/cds = (213,1091)/gb = Z67963/gi = 1067144/ug = Mm 88777/len = 2173/STRA = for | 37.9 | P | 28.9 | P | 24.5 | A |
| 3 | 103302_r_at | Cluster Incl AA866668 ud07a10 r1 Mus musculus cDNA, 5 end/clone = IMAGE-1424426/clone_end = 5/gb = AA866668/ | 372.8 | P | 351.1 | P | 291.6 | P |
| | | gi = 2962113/ug = Mm 21097/len = 537/STRA = for | | | | | | |
| 3 | 92401_at | Cluster Incl U27195 Leukotnene C4 synthase/cds = (36,488)/gb = U27195/gi = 973221/ug = Mm.4088/len = 588/STRA = for | 201.3 | A | 178.8 | M | 155.6 | P |
| 3 | 92519_at | Cluster Incl X74616 Phosphorylase kinase alpha 1/cds = (218,3943)/gb = X74616/gi= 436123/ug = Mm 42254/len = 4130/ | 296.5 | P | 286.7 | P | 242.5 | P |
| | | STRA = for | | | | | | |
| 3 | 94715_at | Cluster Incl K02588 Cytochrome P450, 1a1, aromatic compound inducible/cds = (109,1674)/gb = K02588/gi = 192885/ | 268.7 | A | 296.3 | P | 268.2 | P |
| | | ug = Mm 14089/len = 2619/STRA = for | | | | | | |
| 3 | 97743_at | Cluster Incl AW047461 UI-M-BH1-all-e-12-0-UI s1 Mus musculus cDNA, 3 end/clone = UI-M-BH1-all-e-12-0-UI/ | 1763.9 | P | 1662.6 | P | 1469 | P |
| | | clone_end = 3/gb = AW047461/gi = 5907990/ug = Mm 4016/len = 254/STR = rev | | | | | | |
| 3 | 98847₁₃ at | Cluster Incl U52826 Mus musculus syndecan-3 mRNA, complete cds/cds = (34,1362)/gb = U52826/gi = 1399582/ | 453.7 | A | 468.1 | P | 420.7 | P |
| | | ug = Mm.5059/len = 1495/STRA = for | | | | | | |
| 3 | 97002_f_at | Cluster Incl AJ133427 Mus musculus or37d pseudogene/cds = (0,962)/gb = AJ133427/gi = 5869921/ug = Mm 88844/len = 963/ | 440.4 | P | 377.2 | P | 435 | P |
| | | STRA = for | | | | | | |
| 3 | 102113_f_at | Cluster Incl AI324061 mt75g06 x1 Mus musculus cDNA, 3 end/clone = IMAGE-635770/clone_end = 3/gb = AI324061/ | 348.8 | P | 196.7 | A | 447.8 | P |
| | | gi = 4058490/ug = Mm 42606/len = 258/STRa = rev | | | | | | |
| 3 | 92786_at | Cluster Incl AI626942.vs77b11.x1 Mus musculus cDNA, 3 end/clone = IMAGE-1152285/clone_end= 3/gb= AI626942/ | 191.1 | M | 188.1 | P | 108.3 | A |
| | | gi = 4663742/ug = Mm 12454/len = 306/STRA = for | | | | | | |
| 3 | 93359_at | Cluster Incl AI839743 UI-M-AP0-abe-e-06-0-UI s2 Mus musculus cDNA, 3 end/clone = UI-m-APO-abe-e-06-0-UI/ | 134.3 | P | 123.1 | P | 130.5 | P |
| | | clone_end = 3/gb = AI839743/gi = 5473956/ug = Mm 1893/len = 456/STRA = for | | | | | | |
| 3 | 93592_at | Cluster Incl X82648 M musculus mRNA for apolipoprotein D/cds = (89,658)/gb = X82648/gi = 575656/ug = Mm 2082/len = 903/ | 121.4 | A | 205.3 | P | 129.8 | P |
| | | STRA = for | | | | | | |
| 3 | 94857_at | Cluster Incl U10420 N-methylpurine-DNA glycosylase/cds = (29,1030)/gb = U10420/gi = 500754/ug = Mm 2358/len = 1061/ | 141.1 | P | 81.7 | P | 135.8 | P |
| | | STRA = for | | | | | | |
| 3 | 102070_at | Cluster Incl AW212495 uo89g01 x1 Mus musculus cDNA, 3 end/clone = IMAGE-2649744/clone_end = 3/gb = AW212495/ | 861.8 | P | 820 | P | 722.3 | P |
| | | gi = 6518582/ug = Mm.106/len = 429/STRA = for | | | | | | |
| 3 | 102312_r_at | Cluster Incl AW046728 UI-M-BH1-alt-g-05-0-UI s1 Mus musculus cDNA, 3 end/clone = UI-M-BH1-alt-g-05-0-UI/ | 704.4 | P | 758.1 | A | 654.2 | A |
| | | clone_end = 3/gb = AW046728/gi = 5907297/ug = Mm.10641/len = 302/STRA = for | | | | | | |
| 3 | 102314_at | Cluster Incl M23383 Solute carrier family 2 (facilitated glucose transporter), member 4/cds = (153,1685)/gb = M23383/ | 186.5 | P | 183 | P | 216.7 | P |
| | | gi = 193706/ug = Mm 10661/len = 2521/STRA = for | | | | | | |
| 3 | 103369_at | Cluster Incl AW049142 UI-M-BH1-amq-b-11-0-UI a1 Mus musculus cDNA, 3 end/clone = UI-M-BH1-amq-b-11-0-UI/ | 443.9 | P | 521.9 | P | 467.8 | P |
| | | clone_end = 3/gb = AW049142/gi = 5909671/ug = Mm.18538/len = 446/STRA = rev | | | | | | |
| 3 | 103377_at | Cluster Incl AW259788 um81b12 y1 Mus musculus cDNA, 5 end/clone = IMAGE-2317727/clone_end = 5/gb = AW259788/ | 183.1 | P | 145.9 | P | 128.4 | M |
| | | gi = 6632769/ug = Mm.18600/len = 507/STRA = for | | | | | | |
| 3 | 103838_at | Cluster Incl AB010144 Mus musculus gene for mitsugumin29/cds = (183,977)/gb = AB010144/gi = 3761106/ug = Mm 20942/ | 580.1 | P | 405.5 | P | 372.9 | P |
| | | len = 3248/STRA = for | | | | | | |
| 3 | 94977_at | Cluster Incl X15373 Inositol 1,4,5-triphosphate receptor 1/cds = (328,8577)/gb = X15373/gi = 53568/ug = Mm 2726/len = 9841/ | 32.1 | P | 43.3 | P | 13.9 | A |
| | | STRA = for | | | | | | |
| 3 | 97403_at | Cluster Incl AW045743 UI-M-BH1-akq-h-05-0-UI s1 Mus musculus cDNA, 3 end/clone = UI-M-BH1-akq-h-05-0-UI/ | 281 | P | 284.1 | P | 322.9 | P |
| | | clone_end = 3/gb = AW045743/gi = 5906272/ug = Mm 29900/len = 257/STRA = for | | | | | | |
| 3 | 98513_at | Cluster Incl AI851821: UI-M-BH0-ais-d-03-0-UI s1 Mus musculus cDNA, 3 end/clone = UI-M-BH0-ais-d-03-0-UI/ | 453.4 | P | 435.9 | P | 530.1 | P |
| | | clone_end = 3/gb = AI851821/gi = 5495727/ug = Mm 3600/len = 420/STRA = for | | | | | | |
| 3 | 99050_at | Cluster Incl U36579 Caveolin 3/cds = (121,576)/gb = U36579/gi = 1155228/ug = Mm 3924/len = 1000/STRA = for | 652.4 | P | 564.8 | A | 1001.8 | P |
| 3 | 102639_at | Cluster Incl AB011451 Mus musculus mRNA for N-acetylgucosamine-6-O-sulfotransferase, complete cds/cds = (469,1920)/ | 107.3 | P | 91.6 | A | 129.5 | P |
| | | gb = AB011451/gi = 3402294/ug = Mm.12968/len = 2150/STRA = for | | | | | | |
| 3 | 103212_at | Cluster Incl AI314784 uj35c08 x1 Mus musculus cDNA, 3 end/clone = IMAGE-1921934/clone_end = 3/gb = AI314784/ | 426.8 | P | 287.8 | P | 118.2 | M |
| | | gi = 4030051/ug = Mm 19726/len = 494/STRA = for | | | | | | |
| 3 | 104678_at | Cluster Incl U19860 Growth arrest specific 7/cds = (130,1395)/gb = U19860/gi = 1154621/ug = Mm.3257/len = 2888/ | 155.4 | P | 141.8 | M | 164.8 | P |
| | | STRA = for | | | | | | |
| 3 | 92323_at | Cluster Incl Y13439 Mus musculus mRNA for stress activated protein kinase-3/cds = (167,1270)/gb = Y13439/gi = 2181949/ | 264.9 | P | 147.4 | P | 226.6 | A |
| | | ug = Mm 38343/len = 1553/STRA = for | | | | | | |
| 3 | 92387_at | Cluster Incl L18868 Thromboxane A synthase 1, platelet/cds = (38,1839)/gb = L18868/gi = 1763715/ug = Mm.4054/len = 1847/ | 871.7 | P | 891.9 | A | 590.1 | A |
| | | STRA = rev | | | | | | |
| 3 | 92535_at | Cluster Incl L12147 Earty B-cell factor/cds = (45,1820)/gb = L12147/gi = 309208/ug = Mm 4366/len = 2248/STRA = for | 257 | P | 154.2 | A | 102.6 | A |
| 3 | 93893_f_at | Cluster Incl U56404: Mus musculus strain C57BL/8 natural killer cell receptor Ly-49C mRNA, complete cds/cds = (49,849)/ | 96.8 | P | 103.1 | P | 89 | A |
| | | gb = U56404/gi = 1336030/ug = Mm 89187/len = 907/STRA = for | | | | | | |
| 3 | 94757_at | Cluster Incl M60493 Cystic fibrosis transmembrane conductance regulator homolog/cds = (138,4568)/gb = M60493/ | 109.5 | P | 107.2 | P | 108.5 | M |
| | | gi = 192831/ug = Mm.15621/len = 6304/STRA = for | | | | | | |
| 3 | 95358_at | Cluster Incl AI843864 UI-M-AK1-aeu-c-11-0-UI s1 Mus musculus cDNA, 3 end/clone = UI-M-AK1-aeu-c-11-0-UI/ | 266.1 | A | 411.9 | P | 333.5 | P |
| | | clone_end = 3/gb = AI843864/gi = 5478077/ug = Mm 21518/len = 341/STRA = for | | | | | | |
| 3 | 97158_at | Cluster Incl AI840785 UI-M-AM0-adn-h-05-0-UI.s1 Mus musculus cDNA, 3 end/clone = UI-M-AM0-adn-h-05-0-UI/ | 325.5 | P | 313.8 | A | 299.3 | P |
| | | clone_end = 3/gb = AI840785/gi = 5474998/ug = Mm.35235/len = 324/STRA = rev | | | | | | |
| 3 | 97761_f_at | Cluster Incl U10094 Killer cell lectin-like receptor, subfamily A, member 7/cds = (70,873)/gb = U10094/gi = 533495/ | 301.2 | P | 285.4 | A | 210.9 | P |
| | | ug = Mm 4131/len = 1075/STRA = for | | | | | | |
| 3 | 98782_at | Cluster Incl D38613: Mouse 921-L mRNA for presynaptic protein, complete cds/cds = (84,488)/gb = D38613/gi = 1041029/ | 488.8 | P | 418.5 | A | 361.1 | P |
| | | ug = Mm.4705/len = 1080/STRA = for | | | | | | |
| 3 | 99435_at | Cluster Incl Z22703 Fibroblast growth factor 7/cds = (0,584)/gb = Z22703/gi = 297755/ug = Mm 57177/len = 585/STRA = for | 240.6 | P | 179.1 | A | 210.1 | P |
| 3 | 101684_r_at | Cluster Incl X67863 M musculus T2 mRNA/cds = (174,731)/gb = X67863/gi = 296381/ug = Mm 35868/len = 1263/STRA = for | 803.1 | P | 871.3 | P | 767.9 | P |
| 3 | 93294_at | Cluster Incl M70642: Fibroblast Inducible secreted protein/cds = (137,1183)/gb = M70642/gi = 193315/ug = Mm.1810/ | 169.6 | P | 75.5 | A | 123.6 | P |
| | | len = 2267/STRA = for | | | | | | |
| 3 | 93532_at | Cluster Incl J04992 Troponin I, skeletal, fast 2/cds = (23,571)/gb = J04992/gi = 202164/ug = Mm 1984/len = 667/STRA = for | 738.8 | A | 412.2 | P | 696.2 | P |
| 3 | 95401_at | Cluster Incl AW050156: UI-M-BH1-ano-g-04-0-UI s1 Mus musculus cDNA, 3 end/clone = UI-M-BH1-ano-g-04-0-UI/ | 810.3 | P | 792 | A | 758.8 | A |
| | | clone_end = 3/gb = AW050156/gi = 5910685/ug = Mm.24195/len = 443/STRA = for | | | | | | |
| 3 | 98106_at | Cluster Incl U69898 Mus musculus translocase of inner mitochondrial membrane (Tim44) mRNA, nuclear gene encoding | 711.2 | P | 658.9 | A | 692.3 | A |
| | | mitochondrial protein, complete cds/cds = (8,1366)/gb = U69898/gi = 2351409/ug = Mm.34791/len = 1764/STRA = for | | | | | | |
| 3 | 103815_at | Cluster Incl AI852311 UI-M-BH0-aje-g-04-0-UI s1 Mus musculus cDNA, 3 end/clone = UI-M-BH0-aje-g-04-0-UI/ | 192.3 | A | 211.6 | P | 194.8 | P |
| | | clone_end = 3/gb = AI852311/gi = 5496217/ug = Mm.20901/len = 471/STRA = for | | | | | | |
| 3 | 94442_s_at | Cluster Incl AF030001: Mus musculus major histocompatibility locus class III region-butyrophilin-like protein gene, partial | 308.7 | P | 353.4 | A | 235.5 | P |
| | | cds, Notch4, PBX2, RAGE, lysophatidic acid acyl transferase-alpha, palmitoyl-protein thioesterase 2 (PPT2), CREB-RP, | | | | | | |
| | | and tenascin X/STRA = for | | | | | | |
| 3 | 97336_at | Cluster Incl AJ131851: Mus musculus mRNA for cathepsin F/cds = (16,1404)/gb = AJ131851/gi = 4826564/ug = Mm.29561 | 187.9 | P | 219.5 | P | 278 | P |
| | | len = 1784/STRA = for | | | | | | |
| 3 | 97379_at | Cluster Incl D42083: Mouse mRNA for fructose 1,6-bisphosphatase, complete cds/cds = (19,1083)/gb = D42083/gi = 575935/ | 134.8 | M | 128.2 | P | 133.5 | A |
| | | ug = Mm 2974/len = 1242/STRA = for | | | | | | |
| 3 | 98498_at | Cluster Incl D86353: Mouse mRNA for Mch3, complete cds/cds = (52,963)/gb = D86353/gi = 1945545/ug = Mm 35687/ | 142.6 | P | 109 | P | 91.1 | A |
| | | len = 964/STRA = for | | | | | | |
| 3 | 98983_at | Cluster Incl U16163 Mus musculus prolyl 4-hydroxylase alpha(II)-subunit mRNA, complete cds/cds = (150,1783)/ | 220.6 | P | 73.4 | M | 235.2 | A |
| | | gb = U16163/gi = 836899/ug = Mm 3705/len = 2153/STRA = for | | | | | | |
| 3 | 99044_at | Cluster Incl AI847547 UI-M-AM1-afv-f-02-0-UI.s1 Mus musculus cDNA, 3 end/clone = UI-M-AM1-afv-f-02-0-UI/ | 442.9 | M | 377.8 | A | 138.4 | P |
| | | clone_end = 3/gb = AI847547/gi = 5491453/ug = Mm 39099/len = 385/STRA = for | | | | | | |
| 3 | 99993_at | Cluster Incl U77083 Leucine arylaminopeptidase 1, intestinal/cds = (33,2933)/gb = U77083/gi = 1674500/ug = Mm.4487/ | 250.6 | A | 222.8 | A | 319.3 | P |
| | | len = 3367/STRA = for | | | | | | |
| 3 | 103959_at | Cluster Incl AI605405 vn78f02.x1 Mus musculus cDNA, 3 end/clone = IMAGE-1038075/clone_end = 3/gb = AI605405/ | 253.5 | A | 236 | P | 218.5 | A |
| | | gi = 4614572/ug = Mm 25582/len = 331/STRA = rev | | | | | | |
| 3 | 93423_at | Cluster Incl AI844772 UI-M-AL1-ahq-f-11-0-UI s1 Mus musculus cDNA, 3 end/clone = UI-M-AL1-ahq-f-11-0-UI/ | 257.8 | P | 213.6 | A | 170.1 | A |
| | | clone_end = 3/gb = AI844772/gi = 5488678/ug = Mm.6471/len = 297/STRA = rev | | | | | | |
| 3 | 93618_at | Cluster Incl AF026489: Beta-spectrin 3/cds = (0,47)/gb = AF026489/gi = 2511783/ug = Mm 7363/len = 696/STRA = for | 343.1 | A | 444.7 | A | 439.6 | P |
| 3 | 93700_at | Cluster Incl AI838022 UI-M-AP0-abi-h-01-0-UI s1 Mus musculus cDNA, 3 end/clone = UI-M-AP0-abl-h-01-0-UI/ | 160.5 | P | 42.6 | P | 227.1 | A |
| | | clone_end = 3/gb = AI838022/gl = 5472235/ug = Mm.8612/len = 226/STRA = for | | | | | | |
| 3 | 97776_at | Cluster Incl X55674 M musculus mRNA for D2A dopamine receptor/cds = (129,1463)/gb = X55674/gi = 50648/ | 229.3 | M | 213.2 | P | 150.2 | M |
| | | ug = Mm 41970/len = 2545/STRA = rev | | | | | | |
| 3 | 98328_at | Cluster Incl U02602: Thyroid stimulating hormone, receptor/cds = (74,2368)/gb = U02602/gi = 575923/ug = Mm 4254/ | 74.3 | A | 47.1 | A | 100.3 | P |
| | | len = 2512/STRA = for | | | | | | |
| 3 | 93606_s_at | Cluster Incl AB021966: Mus musculus mRNA for adhesion protein RA175C, complete cds/cds = (0,920)/gb = AB021966 | 188.4 | M | 197.1 | P | 210.9 | P |
| | | gi = 6525254/ug = Mm 20921/len = 921/STRA = for | | | | | | |
| 3 | 96099_at | Cluster Incl X56502 Casein kinase II, beta subunit/cds = (121,788)/gb = X56502/gi = 50420/ug = Mm 26789/len = 944/ | 1004.1 | A | 891.4 | P | 806.7 | A |
| | | STRA = for | | | | | | |
| 3 | 97242_at | Cluster Incl AI849011 UI-M-AJ1-ahd-g-01-0-UI s1 Mus musculus cDNA, 3 end/clone = UI-M-AJ1-ahd-g-01-0-UI/ | 740.4 | P | 495.2 | A | 725.6 | P |
| | | clone_end = 3/gb = AI849011/gi = 5492917/ug = Mm.2781/len= 467/STRA = for | | | | | | |
| 3 | 95068_at | Cluster Incl AA763945: vv44b 12.r1 Mus musculus cDNA, 5 end/clone = IMAGE-1225247/clone_end = 5/gb = AA763945/ | 190.6 | P | 179.4 | A | 166.2 | A |
| | | gi = 2811467/ug = Mm.29186/len = 437/STRA = for | | | | | | |
| 3 | 98452_at | Cluster Incl D88689: Mus musculus mRNA for fit-1, complete cds/cds = (251,4252)/gb = D88689/gi = 2809068/ug = Mm.3464/ | 163.4 | P | 280.3 | P | 181.1 | P |
| | | len = 6275/STRA = for | | | | | | |
| 3 | 98482_at | Cluster Incl X78936 Parathyroid hormone receptor/cds = (56,1831)/gb = X78936/gi = 474828/ug = Mm 3542/len = 1969/ | 223.2 | P | 92.3 | M | 144.2 | A |
| | | STRA = for | | | | | | |
| 3 | 99077_at | Cluster Incl X07750.Thyroid hormone receptor alpha/cds = (0,1232)/gb = X07750/gi = 50387/ug = Mm 4015/len = 1233/ | 231.2 | A | 275.3 | A | 226.1 | P |
| | | STRA = for | | | | | | |
| 3 | 103935_at | Cluster Incl AI504474 vo44e10 Mus musculus cDNA, 3 end/clone = IMAGE-1052778/clone_end = 3/gb = AI504474/ | 320.2 | P | 332.2 | M | 297.8 | A |
| | | gi = 4402325/ug = Mm.25209/len = 265/STRA = rev | | | | | | |
| 3 | 93919_at | Cluster Incl AB020741: Mus musculus NRK mRNA for NIK-related kinase, complete cds/cds = (247,4614)/gb = AB020741/ | 58.2 | P | 26.2 | A | 62.2 | P |
| | | gi = 6009518/ug = Mm 89850/len = 5481/STRA = for | | | | | | |
| 3 | 95988_at | Cluster Incl C79529.C79529 Mus musculus cDNA, 3 end/clone = J0067F08/clone_end = 3/gb = C79529/gi = 2519859/ | 682.2 | A | 472.7 | A | 359.5 | M |
| | | ug = Mm.25039/len = 802/STRA = rev | | | | | | |
| 3 | 98841_at | Cluster Incl M65287 Activin receptor IIA/cds = (70,1611)/gb = M65287/gi = 191663/ug = Mm 5041/len = 2563/STRA = for | 436.1 | A | 350.9 | P | 290.9 | P |
| 3 | 99815_at | Cluster Incl D50096 Histamine receptor H 2/cds = (0,1076)/gb = D50096/gi = 1731686/ug = Mm.57243/len = 1077/STRA = for | 303.6 | M | 243.7 | P | 257.6 | A |
| 3 | 101343_at | Cluster Incl AF020263: Mus musculus EWS/FLI1 activated transcript 2 (EAT-2) mRNA, complete cds/cds = (0,398)/ | 69.8 | M | 226.5 | P | 241.4 | A |
| | | gb = AF020263/gi = 2429353/ug = Mm.57009/len = 399/STRA = for | | | | | | |
| 3 | 100697_at | Cluster Incl X59358 Paired box gene 3/cds = (297,1736)/gb = X59358/gi = 53591/ug = Mm 1371/len = 2347/STRA = for | 183.3 | A | 141.2 | P | 177.4 | A |
| 3 | AFFX-DapX- | B Subtillis Dap 5 prime | 81.8 | A | 82.8 | P | 90.2 | A |
| | 5_at | | | | | | | |
| 3 | 93012-at | Cluster Incl X81464: Translin/cds = (66,752)/gb = X81464/gi = 971555/ug = Mm 14644/len = 991/STRA = for | 141.7 | P | 167.6 | P | 81.1 | A |
| 3 | 96685_at | Cluster Incl AW121774: UI-M-BH2.3-aoe-f-12-0-UI.s1 Mus musculus cDNA, 3 end/clone = UI-M-BH2.3-aoe-f-12-0-UI/ | 560.5 | A | 478.5 | P | 457.5 | P |
| | | clone_end = 3/gb = AW121774/gi = 6097122/ug = Mm. 27472/len = 196/STRA = for | | | | | | |
| 3 | 95113_at | Cluster Incl AW124257: UI-M-BH2.1-aph-g-07-0-UI.s1 Mus musculus cDNA, 3 end/clone = UI-M-BH2, 1-aph-g-07-0-UI/ | 55.1 | M | 86.8 | P | −5.6 | A |
| | | clone_end = 3/gb = AW124257/gi = 6099787/ug = Mm 29372/len = 377/STRA = for | | | | | | |
| 3 | 101034_at | Cluster Incl U07617 Growth factor receptor bound protein 2/cds = (80,733)/gb = U07617/gi = 464004/ug = Mm 6900/ | 385.3 | M | 320.3 | M | 222.2 | M |
| | | len = 2362/STRA = for | | | | | | |
| 3 | 99511_at | Cluster Incl X53532 Protein kinase C, beta/cds = (48,2069)/gb = X53532/gi = 53692/ug = Mm 4182/len = 2974/STRA = for | 135.1 | A | 237.2 | P | 80.4 | A |
| 3 | 102958_at | Cluster Incl U82441 Early B-cell factor 2/cds = (23,1750)/gb = U82441/gi = 3757777/ug = Mm 179/len = 1844/STRA = for | 498.5 | A | 333.1 | M | 482.7 | A |
| 3 | 103240_f_at | Cluster Incl AF017258 Mus musculus ribonuclease 3 precursor (mR-3) gene, complete cds/cds = (0,470)/gb = AF017258/ | 139.1 | A | 229.7 | P | 182.6 | P |
| | | gi = 2407237/ug = Mm 20370/len = 471/STRA = for | | | | | | |
| 3 | 103498_at | Cluster Incl L38613 Mus musculus glucagon receptor mRNA, complete cds/cds = (94,1551)/gb = L38613/gi = 603463/ | 508.8 | A | 510.5 | M | 454.1 | M |
| | | ug = Mm 22329/len = 1944/STRA = for | | | | | | |
| 3 | 103700_r_at | Cluster Incl AI646838 mt86b06.y1 Mus musculus cDNA, 5 end/clone = IMAGE-636755/clone_end = 5/gb = AI646638/ | 373.5 | P | 454.1 | A | 369 | A |
| | | gi = 4725113/ug = Mm 24029/len = 294/STRA = rev | | | | | | |
| 3 | 104421_at | Cluster Incl U87147 Mus musculus flavin-containing monooxygenase 3 mRNA, complete cds/cds = (37,1641)/gb = U87147/ | 212.6 | P | 111.1 | A | 188.2 | A |
| | | gi = 1841861/ug = Mm 2900/len = 2020/STRA = for | | | | | | |
| 3 | 92977_s_at | Cluster Incl X75330 H6 homeo box 3/cds = (307,1683)/gb = X75330/gi = 406586/ug = Mm 5016/len = 1972/STRA = for | 230.4 | A | 163.5 | A | 127.5 | A |
| 3 | 93710_at | Cluster Incl M37759: Mouse serine 1 ultra high sulfur protein gene, complete cds/cds = (0,692)/gb = M37759/gi = 200961/ | 661.2 | A | 735 | P | 188.8 | M |
| | | ug = Mm 86481/len = 693/STRA = for | | | | | | |
| 3 | 96533_at | Cluster Incl AI508931 vc48e10 y1 Mus musculus cDNA, 5 end/clone = IMAGE-777834/clone_end = 5/gb = AI508931/ | 57.2 | A | 86.5 | P | 87.6 | P |
| | | gi = 4407836/ug = Mm 27464/len = 318/STRA = for | | | | | | |
| 3 | 96583_at | Cluster Incl AI414051 ma05b11.x1 Mus musculus cDNA, 3 end/clone = IMAGE-303645/clone_end = 3/gb = AI414051/ | 32.4 | M | 44.5 | P | 13.4 | P |
| | | gi = 4257555/ug = Mm 29393/len = 473/STRA = rev | | | | | | |
| 3 | 101333_at | Cluster Incl U73819 Mus musculus polypeptide GalNAc transferase-T4 (ppGaNTase-T4) mRNA, complete cds/ | 33.7 | A | 14.3 | P | −17.6 | P |
| | | cds = (0,1736)/gb = U73819/gi = 2121219/ug = Mm 56991/len = 1737/STRA = for | | | | | | |
| 3 | 96968_at | Cluster Incl AB024521 Mus musculus mRNA for limitin, complete cds/cds = (160,708)/gb = AB024521/gi = 5821277/ | 518.1 | M | 517.7 | M | 386.3 | P |
| | | ug = Mm 88804/len = 997/STRA = for | | | | | | |
| 4 | AFFX-BioC- | E Coli BioC 3 prime | 767.7 | P | 1267.8 | P | 1278.4 | P |
| | 3_at | | | | | | | |
| 4 | 92547_at | Cluster Incl AB011081 Mus musculus mRNA for huntingtin interacting protein-2, complete cds/cds = (161,763)/ | 563.9 | P | 586.8 | P | 572.4 | P |
| | | gb = AB011081/gi = 2897817/ug = Mm.10082/len = 955/STRA = for | | | | | | |
| 4 | 92622_at | Cluster Incl AI851064 UI-M-BH0-ajv-h-03-0-UI a1 Mus musculus cDNA, 3 end/clone = UI-M-BH0-ajv-h-03-0-UI/ | 405.4 | P | 494.9 | P | 527.5 | P |
| | | clone_end = 3/gb = AI851064/gi = 5494970/ug = Mm 11112/len = 316/STRA = for | | | | | | |
| 4 | 92782_at | Cluster Incl U39074 Thymopoietin/cds = (237,1595)/gb = U39074/gi = 1335840/ug = Mm.124/len = 2170/STRA = for | 238.6 | P | 232.4 | P | 112.2 | P |
| 4 | 92808_f_at | Cluster Incl X17069 Mouse mRNA for protein with homology to transition protein 2 (TP2)/cds = (123,1358)/gb = X17069/ | 799.5 | P | 878.5 | P | 940.1 | P |
| | | gi = 54875/ug = Mm 12758/len = 2026/STRA = for | | | | | | |
| 4 | 92850_at | Cluster Incl AI836446 UI-M-AI0-aak-d-12-0-UI s1 Mus musculus cDNA, 3 end/clone = UI-M-AI0-aak-d-12-0-UI/ | 884.3 | P | 980.7 | P | 816.2 | P |
| | | clone_end = 3/gb = AI836446/gi = 5470659/ug = Mm 13705/len = 451/STRA = for | | | | | | |
| 4 | 93014_at | Cluster Incl Y17223 Mus musculus mRNA for F1F0-ATP synthase, g subunit/cds = (11,322)/gb = Y17223/gi = 3123645/ | 3046.8 | P | 3320.4 | P | 3868.9 | P |
| | | ug = Mm 14663/len = 420/STRA = for | | | | | | |
| 4 | 93028_at | Cluster Incl X58196 H19 fetal liver mRNA/cds = UNKNOWN/gb = X58196/gi = 51131/ug = Mm 14802/len = 1899/STRA = for | 3031.6 | P | 3655.5 | P | 3722.8 | P |
| 4 | 93064_at | Cluster Incl AF035207 Mus musculus Nip21 mRNA, complete cds/cds = (133,1113)/gb = AF035207/gi = 2911347/ | 203.6 | P | 220.5 | P | 274.1 | P |
| | | ug = Mm.1561/len = 2213/STRA = for | | | | | | |
| 4 | 93293_at | Cluster Incl M27844 Calmodulin/cds = (0,449)/gb = M27844/gi = 192364/ug = Mm 18041/len = 450/STRA = for | 4410.1 | P | 4666.1 | P | 4240.1 | P |
| 4 | 93306_at | Cluster Incl U51196: Mus musculus APC-binding protein EB1 homolog mRNA, complete cds/cds = (66,872)/gb = U51196/ | 1165.2 | P | 1302.7 | P | 1107.8 | P |
| | | gi = 1256433/ug = Mm.18418/len = 1203/STRA = for | | | | | | |
| 4 | 93354_at | Cluster Incl Z22661 Apolipoprotein Cl/cds = (49,315)/gb = Z22661/gi = 410495/ug = Mm 189/len = 408/STRA = for | 920.8 | P | 898 | P | 851.3 | P |
| 4 | 93365_s_at | Cluster Incl AI838149 UI-M-AL.0-abw-e-03-0-UI s1 Mus musculus cDNA, 3 end/clone = UI-M-AL0-abw-e-03-0-UI/ | 504.4 | P | 444.1 | P | 465.6 | P |
| | | clone_end = 3/gb = AI838149/gi = 5472362/ug = Mm 18972/len = 402/STRA = for | | | | | | |
| 4 | 93366_r_at | Cluster Incl AI838149 UI-M-AL.0-abw-e-03-0-UI s1 Mus musculus cDNA, 3 end/clone = UI-M-AL0-abw-e-03-0-UI/ | 731.7 | P | 663.5 | P | 656.5 | P |
| | | clone_end = 3/gb = AI838149/gi = 5472362/ug = Mm 18972/len = 402/STRA = for | | | | | | |
| 4 | 93579_at | Cluster Incl AW121613 UI-M-BH2 2-aoq-d-12-0-UI s1 Mus musculus cDNA, 3 end/clone = UI-M-BH2 2-aoq-d-12-0-UI/ | 427.3 | P | 563 | P | 622.2 | P |
| | | clone_end = 3/gb = AW121613/gi = 6096946/ug = Mm 2053/len = 135/STRA = for | | | | | | |
| 4 | 93800_f_at | Cluster Incl AB013607 Mus musculus mRNA for c29, complete cds/cds = (21,1367)/gb = AB013607/gi = 3868801/ | 1899 | P | 3885.1 | P | 3084.9 | P |
| | | ug = Mm 21408/len = 1539/STRA = for | | | | | | |
| 4 | 93810_at | Cluster Incl X68378 Cathepsin D/cds = (0,1232)/gb = X68378/gi = 50302/ug = Mm 2147/len = 1233/STRA = for | 1565.1 | P | 1851 | P | 2671.5 | P |
| 4 | 93822_at | Cluster Incl X73331 M musculus mRNA for ribosomal protein L37a/cds = (18,296)/gb = X73331/gi = 312413/ug = Mm 21529/ | 1557.2 | P | 1393.6 | P | 1323.4 | P |
| | | len = 349/STRA = for | | | | | | |
| 4 | 93844_at | Cluster Incl AW061302 UI-M-BH1-anw-c-09-0-UI s1 Mus musculus cDNA, 3 end/clone = UI-M-BH1-anw-c-09-0-UI/ | 3031.8 | P | 3130.9 | P | 4259.3 | P |
| | | clone_end = 3/gb = AW061302/gi = 6009053/ug = Mm 21625/len = 321/STRA = for | | | | | | |
| 4 | 93980_at | Cluster Incl AW124520 UI-M-BH2, 1-apo-f-11-0-UI.s1 Mus musculus cDNA, 3 end/clone = UI-M-BH2 1-apo-f-11-0-UI/ | 82.8 | P | 135.2 | P | 133 | P |
| | | clone_end = 3/gb = AW124520/gi = 6100050/ug = Mm 21693/len = 511/STRA = for | | | | | | |
| 4 | 93987_f_at | Cluster Incl AW121568: UI-M-BH2 2-aop-g-06-0-UI s1 Mus musculus cDNA, 3 end/clone = UI-M-BH2 2-aop-g-06-0-UI/ | 4458.7 | P | 4873.1 | P | 4704.2 | P |
| | | clone_end = 3/gb = AW121568/gi = 6096901/ug = Mm 21724/len = 329/STRA = for | | | | | | |
| 4 | 94034_at | Cluster Incl AI839882: UI-M-AH0-acw-b-11-0-UI s1 Mus musculus cDNA, 3 end/clone = UI-M-AH0-acw-b-11-0-UI/ | 234.9 | P | 385.6 | P | 387 | P |
| | | clone_end = 3/gb = AI839882/gi = 5474095/ug = Mm 21911/len = 353/STRA = for | | | | | | |
| 4 | 94042_f_at | Cluster Incl AI843937: UI-M-AP1-agh-b-10-0-UI a1 Mus musculus cDNA, 3 end/clone = UI-M-AP1-agh-b-10-0-UI/ | 4489.3 | P | 4594.9 | P | 4116.8 | P |
| | | clone_end = 3/gb = AI843937/gi = 5478150/ug = Mm 21955/len = 356/STRA = for | | | | | | |
| 4 | 94056_at | Cluster Incl M21285 Stearoyl-coenzyme A desaturase 1/cds = (0,1067)/gb = M21285/gi = 200949/ug = Mm 22026/len = 1068/ | 1619.7 | P | 3554.1 | P | 2403.1 | P |
| | | STRA = for | | | | | | |
| 4 | 94057_g_at | Cluster Incl M21285: Stearoyl-coenzyme A desaturase 1/cds = (0,1067)/gb = M21285/gi = 200949/ug = Mm.22026/len = 1068/ | 772.9 | P | 1851 | P | 1233.8 | P |
| | | STRA = for | | | | | | |
| 4 | 94218_at | Cluster Incl M20130: Mouse t complex polypeptide 1 (Tcp-1-a) mRNA, 3 end/cds = (0,714)/gb = M20130/gi = 201722/ | 194.1 | P | 290.5 | P | 276.4 | P |
| | | ug = Mm 2223/len = 863/STRA = for | | | | | | |
| 4 | 94276_at | Cluster Incl AF064635: Mus musculus putative ateroid dehydrogenase (KIK-I) mRNA, complete cds/cds = (19,957)/ | 761.5 | P | 1287.3 | P | 887.3 | P |
| | | gb = AF064635/gi = 3142701/ug = Mm.22505/len = 978/STRA = for | | | | | | |
| 4 | 94312_at | Cluster Incl AI180784: ub76c07.r1 Mus musculus cDNA, 5 and/clone = IMAGE-1383660/clone_end = 5/gb = AI180784/ | 594.2 | P | 552.1 | P | 506.6 | P |
| | | gi = 3731422/ug = Mm 22635/len = 360/STRA = rev | | | | | | |
| 4 | 94819_f_at | Cluster Incl AF005886: Mus musculus cyclin I mRNA, complete cds/cds = (286,1419)/gb = AF005888/gi = 4101686/ | 1614.8 | P | 1475.8 | P | 1630 | P |
| | | ug = Mm 22711/len = 1548/STRA = for | | | | | | |
| 4 | 95437_at | Cluster Incl AI836139 UI-M-AQ0-aai-b-06-0-UI.a2 Mus musculus cDNA, 3 end/clone = UI-M-AQ0-aai-b-06-0-UI/ | 469.6 | P | 480.5 | P | 600 | P |
| | | clone_end = 3/gb = AI836139/gi = 5470352/ug = Mm 24538/len = 395/STRA = for | | | | | | |
| 4 | 95442_at | Cluster Incl AI835706: UI-M-AIO-aan-b-08-0-UI s1 Mus musculus cDNA, 3 end/clone = UI-M-AI0-aan-b-08-0-UI/ | 437.5 | P | 400.5 | P | 343.4 | P |
| | | clone_end = 3/gb = AI835706/gi = 5469919/ug = Mm 24571/len = 329/STRA = for | | | | | | |
| 4 | 95457_at | Cluster Incl AI854214 UI-M-BH0-ajz-e-09-0-UI s1 Mus musculus cDNA, 3 end/clone = UI-M-BH0-ajz-e-09-0-UI/ | 565.9 | P | 744.2 | P | 745 | P |
| | | clone_end = 3/gb = AI854214/gi = 5498120/ug = Mm 24703/len = 647/STRA = for | | | | | | |
| 4 | 95470_at | Cluster Incl AI846025: UI-M-AP1-agi-g-04-0-UI s1 Mus musculus cDNA, 3 end/clone = UI-M-AP1-agi-g-04-0-UI/ | 425.4 | P | 353.5 | P | 519.7 | P |
| | | clone_end = 3/gb = AI846025/gi = 5489931/ug = Mm 24789/len = 451/STRA = for | | | | | | |
| 4 | 96027_at | Cluster Incl AW120546: UI-M-BH1-ana-e-02-0-UI.s2 Mus musculus cDNA, 3 end/clone = UI-M-BH1-ana-e-02-0-UI/ | 175 | P | 216.9 | P | 239.5 | P |
| | | clone_end = 3/gb = AW120546/gi = 6095879/ug = Mm 25732/len = 473/STRA = for | | | | | | |
| 4 | 96050_at | Cluster Incl AJ011740 Mus musculus mRNA for integrase interacter 1a protein (INI1A)/cds = (193,1350)/gb = AJ011740/ | 572.4 | P | 590.6 | P | 569.9 | P |
| | | gi = 4138222/ug = Mm 26116/len = 1622/STRA = for | | | | | | |
| 4 | 96068_at | Cluster Incl AI848821: UI-M-AJ1-ahb-a-11-0-UI s1 Mus musculus cDNA, 3 end/clone = UI-M-AJ1-ahb-a-11-0-UI/ | 236.6 | P | 174.6 | P | 252.6 | P |
| | | clone_end = 3/gb = AI848821/gi = 5492727/ug = Mm 26418/len = 401/STRA = for | | | | | | |
| 4 | 96653_at | Cluster Incl AI851762: UI-M-BH0-aim-d-03-0-UI.s1 Mus musculus cDNA, 3 end/clone = UI-M-BH0-aim-d-03-0-UI/ | 1400.1 | P | 2026.2 | P | 1225.9 | P |
| | | clone_end = 3/gb = AI851762/gi = 5495668/ug = Mm 27336/len = 419/STRA = for | | | | | | |
| 4 | 96674_at | Cluster Incl AW123553: UI-M-BH2 1-aqb-f-12-0-UI s1 Mus musculus cDNA, 3 end/clone = UI-M-BH2 1-aqb-f-12-0-UI/ | 616.4 | P | 965.7 | P | 853.2 | P |
| | | clone_end = 3/gb = AW123553/gi = 6099083/ug = Mm 27404/len = 400/STRA = for | | | | | | |
| 4 | 96677_at | Cluster Incl AI849566: UI-M-AH1-agr-c-04-0-UI s1 Mus musculus cDNA, 3 end/clone = UI-M-AH1-agr-c-04-0-UI/ | 582.3 | P | 843.3 | P | 801.5 | P |
| | | clone_end = 3/gb = AI849566/gi = 5493472/ug = Mm 27424/len = 351/STRA = for | | | | | | |
| 4 | 96684_at | Cluster Incl AI846123: UI-M-AN1-afj-a-11-0-UI s2 Mus musculus cDNA, 3 end/clone = UI-M-AN1-afj-a-11-0-UI/ | 216 | P | 193.2 | P | 592.2 | P |
| | | clone_end = 3/gb = AI846123/gi = 5490029/ug = Mm 27453/len = 341/STRA = for | | | | | | |
| 4 | 97206_at | Cluster Incl AW230369: uo62g07 y1 Mus musculus cDNA, 5 end/clone = IMAGE-2647164/clone_end = 5/gb = AW230369/ | 438.5 | P | 658.5 | P | 502.7 | P |
| | | gi = 6559685/ug = Mm 27700/len = 692/STRA = for | | | | | | |
| 4 | 98894_at | Cluster Incl AA867655 vx12e03 r1 Mus musculus cDNA, 5 end/clone = IMAGE-1264252/clone_end = 5/gb = AA867655/ | 513.8 | P | 804.5 | P | 702 | P |
| | | gi = 2963100/ug = Mm 28550/len = 428/STRA = for | | | | | | |
| 4 | 98901_at | Cluster Incl AW047870: UI-M-BH1-afs-f-10-0-UI s1 Mus musculus cDNA, 3 end/clone = UI-M-BH1-afs-f-10-0-UI/ | 543.9 | P | 620.8 | P | 578.9 | P |
| | | clone_end = 3/gb = AW047870/gi = 5908399/ug = Mm 28578/len = 353/STRA = for | | | | | | |
| 4 | 94456_at | Cluster Incl AI841497: UI-M-AH0-acy-d-12-0-UI s1 Mus musculus cDNA, 3 end/clone = UI-M-AH0-acy-d-12-0-UI/ | 1372.5 | P | 1681 | P | 1327.7 | P |
| | | clone_end = 3/gb = AI841497/gi = 5475710/ug = Mm 28805/len = 411/STRA = for | | | | | | |
| 4 | 94466_f_at | Cluster Incl U19891 CCAAT/enhancer binding protein alpha (C/EBP), related sequence 1/cds = (11,3004)/gb = U19891/ | 366.3 | P | 498.8 | P | 528.6 | P |
| | | gi = 790574/ug = Mm 28845/len = 3302/STRA = for | | | | | | |
| 4 | 94528_at | Cluster Incl AI846206 UI-M-AM1-agb-e-11-0-UI s1 Mus musculus cDNA, 3 end/clone = UI-M-AM1-agb-e-11-0-UI/ | 290.9 | P | 248.4 | P | 277.1 | P |
| | | clone_end = 3/gb = AI846206/gi = 5490112/ug = Mm 29037/len = 141/STRA = for | | | | | | |
| 4 | 94548_at | Cluster Incl AI790537 ul02d10 x1 Mus musculus cDNA, 3 end/clone = IMAGE-2064883/clone_end = 3/gb = AI790537/ | 471.3 | P | 506.1 | P | 897.7 | P |
| | | gi = 5338253/ug = Mm.29078/len = 440/STRA = for | | | | | | |
| 4 | 95058_f_at | Cluster Incl AW121984 UI-M-BH2 3-aoj-b-10-0-UI s1 Mus musculus cDNA, 3 end/clone = UI-M-BH2 3-aoj-b-10-0-UI/ | 1412.7 | P | 1493.9 | P | 1736.2 | P |
| | | clone_end = 3/gb = AW121984/gi = 6097345/ug = Mm.29153/len = 390/STRA = for | | | | | | |
| 4 | 95118_r_at | Cluster Incl AI131895 ue97e08 x1 Mus musculus cDNA, 3 end/clone = IMAGE-1499078/clone_end = 3/gb = AI131895/ | 816.2 | P | 610.5 | P | 641.6 | P |
| | | gi = 3601911/ug = Mm.29381/len = 397/STRA = for | | | | | | |
| 4 | 95686_at | Cluster Incl AW047017 UI-M-BH1-alp-h-05-0-UI s1 Mus musculus cDNA, 3 end/clone = UI-M-BH1-alp-h-05-0-UI/ | 149.6 | P | 215.8 | P | 246.3 | P |
| | | clone_end = 3/gb = AW047017/gi = 5907546/ug = Mm.29641/len = 353/STRA = for | | | | | | |
| 4 | 95702_at | Cluster Incl AI461803 uc43c04.x1 Mus musculus cDNA, 3 end/clone = IMAGE-1400742/clone_end = 3/gb = AI461803/ | 564.2 | P | 821.2 | P | 946.1 | P |
| | | gi = 4315833/ug = Mm.29697/len = 481/STRA = for | | | | | | |
| 4 | 95744_at | Cluster Incl U13837: Mus musculus vacuolar adenosine triphosphatase subunit A gene, complete cds/cds = (0,1853)/ | 699.7 | P | 545.1 | P | 629.2 | P |
| | | gb = U13837/gi = 1184658/ug = Mm.29771/len = 1854/STRA = for | | | | | | |
| 4 | 95745_g_at | Cluster Incl U13837 Mus musculus vacuolar adenosine triphosphatase subunit A gene, complete cds/cds = (0,1853)/ | 539.5 | P | 711.4 | P | 665.6 | P |
| | | gb = U13837/gi = 1184658/ug = Mm.29771/len = 1854/STRA = for | | | | | | |
| 4 | 95749_at | Cluster Incl AW122364 UI-M-BH2.2-aow-a-11-0-UI.s1 Mus musculus CDNA, 3 end/clone = UI-M-BH2.2-aow-a-11-0-UI/ | 113 | P | 277.7 | P | 409.6 | P |
| | | clone_end = 3/gb = AW122364/gi = 6097859/ug = Mm 29778/len = 488/STRA = for | | | | | | |
| 4 | 95758_at | Cluster Incl M26270 Stearoyl-coenzyme A desaturase 2/cds = (300,1376)/gb = M26270/gi = 200933/ug = Mm.298/len = 5032/ | 2443 | P | 2020.5 | P | 1966.4 | P |
| | | STRA = for | | | | | | |
| 4 | 96270_at | Cluster Incl AI847092: UI-M-AP1-agn-e-07-0-U1 s1 Mus musculus CDNA, 3 end/clone = UI-M-AP1-agn-e-07-0-UI/ | 1394.3 | P | 1573.7 | P | 1517.7 | P |
| | | clone_end = 3/gb = AI847092/gi = 5490998/ug = Mm 29852/len = 261/STRA = for | | | | | | |
| 4 | 96337_at | Cluster Incl AF033350: Mus musculus CDCREL-1 homolog (Cdcrel-1) MRNA, partial cds/cds = (0,1044)/gb = AF033350/ | 401.8 | P | 373.4 | P | 378.5 | P |
| | | gi = 3986408/ug = Mm 29976/len = 1973/STRA = for | | | | | | |
| 4 | 96343_at | Cluster Incl AI836968 UI-M-AJ0-aaz-c-09-0-UI s1 Mus musculus CDNA, 3 end/clone = UI-M-AJ0-aaz-c-09-0-UI/ | 1799.5 | P | 2053.5 | P | 1930.8 | P |
| | | clone_end = 3/gb = AI836968/gi = 5471181/ug = Mm.29991/len = 445/STRA = for | | | | | | |
| 4 | 96849_at | Cluster Incl AA655369: vs24h12 r1 Mus musculus CDNA, 5 end/clone = IMAGE-1139207/clone_end = 5/gb = AA655369/ | 282 | P | 360.1 | P | 316.6 | P |
| | | gi = 2591523/ug = Mm 30034/len = 545/STRA = for | | | | | | |
| 4 | 96943_at | Cluster Incl AW125234 UI-M-BH2 1-apa-e-0300-UI s1 Mus musculus cDNA, 3 end/clone = UI-M-BH2 1-apa-e-03-0-UI/ | 677.7 | P | 795.2 | P | 927.2 | P |
| | | clone_end = 3/gb = AW125234/gi = 6100764/ug = Mm 30195/len = 310/STRA = for | | | | | | |
| 4 | 97456_at | Cluster Incl AI838021: UI-M-AP0-abl-g-11-0-UI.s1 Mus musculus cDNA, 3 end/clone = UI-M-AP0-abl-g-11-0-UI/ | 538.2 | P | 716.7 | P | 588.1 | P |
| | | clone_end = 3/gb = AI838021/gi = 5472234/ug = Mm.30263/len = 392/STRA = for | | | | | | |
| 4 | 97459_at | Cluster Incl AI851441: UI-M-BH0-akj-e-05-0-UI.s1 Mus musculus cDNA, 3 end/clone = UI-M-BH0-akj-e-05-0-UI/ | 1501.5 | P | 1397 | P | 1672.4 | P |
| | | clone_end = 3/gb = AI851441/gi = 5495347/ug = Mm.30270/len = 338/STRA = for | | | | | | |
| 4 | 97497_at | Cluster Incl Z11886 Notch gene homolog 1, (Drosphila)/cds = (78,7673)/gb = Z11886/gi = 288502/ug = Mm.31255/len = 8064/ | 381.6 | P | 963.3 | P | 894.6 | P |
| | | STRA = for | | | | | | |
| 4 | 97504_at | Cluster Incl M83749 Cyclin D2/cds = (270,1139)/gb = M83749/gi = 192938/ug = Mm 3141/len = 1255/STRA = for | 1258.6 | P | 987.5 | P | 1236.9 | P |
| 4 | 97512_at | Cluster Incl AW226650 um58g10.y1 Mus musculus cDNA, 5 end/clone = IMAGE-2270274/clone_end = 5/gb = AW226650/ | 2100.3 | P | 2197.9 | P | 2602.8 | P |
| | | gi = 6555946/ug = Mm 318/len = 390/STRA = for | | | | | | |
| 4 | 98053_at | Cluster Incl AF058797 Mus musculus 14-3-3 protein beta mRNA, complete cds/cds = (171,911)/gb = AF058797/gi = 3065924/ | 1481.2 | P | 1652.1 | P | 1690.7 | P |
| | | ug = Mm 34319/len = 1022/STRA = for | | | | | | |
| 4 | 98056_at | Cluster Incl AI846214 UI-M-AM1-agb-f-10-0-UI s1 Mus musculus cDNA, 3 end/clone = UI-M-AM1-agb-f-10-0-UI/ | 713.4 | P | 1010.5 | P | 1285.4 | P |
| | | clone _end = 3/gb = AI846214/gi = 5490120/ug = Mm 34346/len = 343/STRA = for | | | | | | |
| 4 | 98077_at | Cluster Incl AA796831: vp15g02 r1 Mus musculus cDNA, 5 end/clone = IMAGE-1068722/clone_end = 5/gb = AA796831/ | 175.7 | P | 345.2 | P | 169.1 | P |
| | | gi = 2859786/ug = Mm 34549/len = 424/STRA = for | | | | | | |
| 4 | 98111_at | Cluster Incl L40406 Heat shock protein, 105 kDa/cds = (139,2718)/gb = L40406/gi = 840651/ug = Mm 34828/len = 3387/ | 264.1 | P | 349.9 | P | 367 | P |
| | | STRA = for | | | | | | |
| 4 | 98112_r_at | Cluster Incl AI839225 UI-M-AK0-adh-d-01-0-UI s1 Mus musculus cDNA, 3 end/clone = UI-M-AK0-adh-d-01-0-UI/ | 271.8 | P | 322.7 | P | 324.6 | P |
| | | clone_end = 3/gb = AI839225/gi = 5473438/ug = Mm 34832/len = 429/STRA = for | | | | | | |
| 4 | 98113_at | Cluster Incl U60824: Mus musculus proteasome beta-subunit C5 (Pamb1) mRNA, allele B, complete cds/cds = (29,751)/ | 2031.4 | P | 2065.6 | P | 2136.6 | P |
| | | gb = U60824/gi = 1698583/ug = Mm 34837/len = 812/STRA = for | | | | | | |
| 4 | 98122_at | Cluster Incl AF074600 Mus musculus LIM domain transcription factor LMO4 mRNA, complete cds/cds = (0,497)/ | 1975 | P | 2019.1 | P | 1485 | P |
| | | gb = AF074600/gi = 3702853/ug = Mm.35004/len = 498/STRA = for | | | | | | |
| 4 | 98532_at | Cluster Incl AF011644 Mus musculus oral tumuor suppressor homolog (Doc-1) mRNA, partial cds/cds = (0,330)/ | 1364.4 | P | 1465 | P | 1480.4 | P |
| | | gb = AF011644/gi = 2305225/ug = Mm 35847/len = 981/STRA = for | | | | | | |
| 4 | 98555_at | Cluster Incl AB008516: Tetratricopeptide repeat domain/cds = (11,5950)/gb = AB008516/gi = 3308963/ug = Mm 3679/ | 530.1 | P | 472.1 | P | 454.3 | P |
| | | len = 7172/STRA = for | | | | | | |
| 4 | 98602_at | Cluster Incl U08110 RAN GTPase activating protein 1/cds = (274,2043)/gb = U08110/gi = 472851/ug = Mm 3833/len = 2981/ | 456.9 | P | 342.4 | P | 374.1 | P |
| | | STRA = for | | | | | | |
| 4 | 99098_at | Cluster Incl AW045533 UI-M-BH1-aks-c-08-0-UI s1 Mus musculus cDNA, 3 end/clone = UI-M-BH1-aks-c-08-0-UI/ | 660.2 | P | 1034.6 | P | 862 | P |
| | | clone_end = 3/gb = AW045533/gi = 5906062/ug = Mm 39472/len = 426/STRA = for | | | | | | |
| 4 | 99128_at | Cluster Incl AI849767 UI-M-AL1-ahn-f-09-0-UI s1 Mus musculus cDNA, 3 end/clone = UI-M-AL1-ahn-f-09-0-UI/ | 2447.4 | P | 2007.9 | P | 2763.7 | P |
| | | clone_end = 3/gb = AI849767/gi = 5493673/ug = Mm 41/len = 362/STRA = for | | | | | | |
| 4 | 99147_at | Cluster Incl AI843686 UI-M-AP1-agh-05-0-UI s1 Mus musculus cDNA, 3 end/clone = UI-M-AP1-agh-g-05-0-UI/ | 811.6 | P | 1235.9 | P | 1459.5 | P |
| | | clone_end = 3/gb = AI843986/gi = 5478199/ug = Mm 41486/len = 246/STRA = for | | | | | | |
| 4 | 99569_at | Cluster Incl AF021836 Mus muscules hair keratin basic 5 (Hb5) mRNA, complete cds/cds = (61,1584)/gb = AF021836/ | 808.1 | P | 1937.7 | P | 1023.3 | P |
| | | gi = 4103155/ug = Mm 42251/len = 2344/STRA = for | | | | | | |
| 4 | 99583_at | Cluster Incl X53451 Glutathione S-transferase, pi 2/cds = (8,640)/gb = X53451/gi = 51124/ug = Mm 426/len = 703/STRA = for | 1407 | P | 965 | P | 1446.8 | P |
| 4 | 99607_at | Cluster Incl Z47088: Transcription elongation factor B (SIII), polypeptide 1 (15 KDa),-like/cds = UNKNOWN/gb = Z47088/ | 1219.5 | P | 1286.6 | P | 1181.6 | P |
| | | gi = 861057/ug = Mm.42944/len = 1219/STRA = for | | | | | | |
| 4 | 99631_f_at | Cluster Incl U08440 Mus musculus Balb/c cytochrome c oxidase subunit VIaL mRNA, complete cds/cds = (72,332)/ | 3945.3 | P | 3327.3 | P | 3616 | P |
| | | gb = U08440/gi = 475706/ug = Mm.43415/len = 521/STRA = for | | | | | | |
| 4 | 99672_at | Cluster Incl Z49916: M.muscules mRNA for CLCN4/cds = (377,2620)/gb = Z49916/gi = 929679/ug = Mm 4408/len = 2739/ | 167.1 | P | 265.2 | P | 260.8 | P |
| | | STRA = for | | | | | | |
| 4 | 99674_at | Cluster Incl AW060297 UI-M-BH1-amv-c-04-0-UI s1 Mus musculus cDNA, 3 end/clone = UI-M-BH1-amv-c-04-0-UI/ | 518.2 | P | 599.6 | P | 528 | P |
| | | clone_end = 3/gb = AW060297/gi = 6008013/ug = Mm 44174/len = 415/STRA = for | | | | | | |
| 4 | 100066_at | Cluster Incl U20892 Phoshonbosylglycinamide formyltransferase/cds = (115,3147)/gb = U20892/gi = 2182233/ug = Mm 4505/ | 385.7 | P | 549.7 | P | 504.5 | P |
| | | len = 3296/STRA = for | | | | | | |
| 4 | 100091_at | Cluster Incl D87990 House mouse, Musculus domesticus mRNA for UDP-galactose transporter related isozyme 1, | 1017.7 | P | 1232.3 | P | 11809.9 | P |
| | | complete cds/cds = (88,1056)/gb = D87990)/gi = 1694638/ug = Mm.4593/len = 1169/STRA = for | | | | | | |
| 4 | 100550_f_at | Cluster Incl AW060422: UI-M-BH1-anj-c-07-0-UI s1 Mus musculus cDNA, 3 end/clone = UI-M-BH1-anj-c-07-0-UI/ | 3483.3 | P | 3775 | P | 3563.2 | P |
| | | clone_end = 3/gb = AW060422/gi = 6008173/ug = Mm 548/len = 373/STRA = for | | | | | | |
| 4 | 100567_at | Cluster Incl M20497 Mouse adipose fatty acid binding protein (422) gene, 5 end/cds = (65,97)/gb = M20497/gi = 191743/ | 934.8 | P | 2507.9 | P | 2345.9 | P |
| | | ug = Mm 582/len = 98/STRA = for | | | | | | |
| 4 | 101044_at | Cluster Incl X13752 Delta-aminolevulinate dehydratase/cds = (6,998)/gb = X13752/gi = 52951/ug = Mm 6988/len = 1080/ | 387.8 | P | 637.7 | P | 527.2 | P |
| | | STRA = for | | | | | | |
| 4 | 101051_at | Cluster Incl AF004941 Mus musculus S100A3 calcuim binding protein mRNA, complete cds/cds = (59,364)/gb = AF004941/ | 649.4 | P | 2902.7 | P | 1623.1 | P |
| | | gi = 3916049/ug = Mm 703/len = 712/STRA = for | | | | | | |
| 4 | 101052_g_at | Cluster Incl AF004941 Mus musculus S100A3 calcuim binding protein mRNA, complete ccds/cds = (59,364)/gb = AF004941/ | 348.8 | P | 1653.3 | P | 525.8 | P |
| | | gi = 3916049/ug = Mm 703/len = 712/STRA = for | | | | | | |
| 4 | 101530_at | Cluster Incl U97079 Mus musculus US-116 KD mRNA, complete cds/cds = (27,2942)/gb = U97079/gi = 2276461/ug = Mm 873/ | 423.9 | P | 510.5 | P | 438 | P |
| | | len = 3271/STRA = for | | | | | | |
| 4 | 101561_at | Cluster Incl K02236 Metallothionein 2/cds = (0,185)/gb = K02236/gi = 199131/ug = Mm.89170/len = 186/STRA = for | 3842 | P | 4848.3 | P | 5061.9 | P |
| 4 | 101569_at | Cluster Incl AB033744 Mus musculus mRNA for type II cytokeratin, complete cds/cds = (51,1625)/gb = AB0033744/ | 1179.1 | P | 4078.3 | P | 2312.6 | P |
| | | gi = 6092074/ug = Mm 89769/len = 2193/STRA = for | | | | | | |
| 4 | 101581_at | Cluster Incl U82122.Ubiqutin conjugating enzyme E3A/cds = (0,2549)/gb = U82122/gi = 1843534/ug = Mm 9002/len = 2550/ | 212.9 | P | 337.8 | P | 303.4 | P |
| | | STRA = for | | | | | | |
| 4 | 101984_at | Cluster Incl AF004591 Mus musculus copper transport protein Atox1 (ATOX1) mRNA, complete cds/cds = (38,244)/ | 3066.9 | P | 2585.4 | P | 3121.1 | P |
| | | gb = AF004591/gi = 2209342/ug = Mm.9703/len = 432/STRA = for | | | | | | |
| 4 | 102009_at | Cluster Incl AI835274 UI-M-AQ0-aab-c-12-0-UI s1 Mus musculus cDNA, 3 end/clone = UI-M-AQ0-aab-c-12-0-UI/ | 213.1 | P | 231.8 | P | 196.9 | P |
| | | clone_end = 3/gb = AI835274/gi = 5469487/ug = Mm.100115/len = 460/STRA = rev | | | | | | |
| 4 | 102056_f_at | Cluster Incl AA839379: vw48g12.r1 Mus musculus cDNA, 5 end/clone = IMAGE-1247110/clone_end = 5/gb = AA839379/ | 234.5 | P | 273.3 | P | 247.2 | P |
| | | gi = 2915474/ug = Mm.10374/len = 500/STRA = for | | | | | | |
| 4 | 102062_at | Cluster Incl U85614: Mus musculus SRG3 mRNA, complete cds/cds = (60,3362)/gb = U85614/gi = 1816634/ug = Mm 1050/ | 418 | P | 394.3 | P | 611.6 | P |
| | | len = 4765/STRA = for | | | | | | |
| 4 | 102069_at | Cluster Incl S78454 Mus musculus metal response element DNA-binding protein M96 mRNA, complete cds/cds = (0,1121)/ | 307.6 | P | 475.3 | P | 439.1 | P |
| | | gb = S78454/gi = 998845/ug = Mm.10563/len = 1517/STRA = for | | | | | | |
| 4 | 102754_at | Cluster Incl Y17267: Mus musculus mRNA for ubiquitin conjugating enzyme/cds = (258,14795)/gb = Y17267/gi = 3319989/ | 179 | P | 327.4 | P | 364.4 | P |
| | | ug = Mm 12921/len = 15475/STRA = for | | | | | | |
| 4 | 102793_at | Cluster Incl D86423 Mus musculus mRNA for HGT keratin, partial cds/cds = (0,163)/gb = D86423/gi = 1944398/ | 134.3 | P | 810.5 | P | 262.6 | P |
| | | ug = Mm.13979/len = 508/STRA = for | | | | | | |
| 4 | 103067_at | Cluster Incl Z48587: M musculus ral-A mRNA/cds = (0,620)/gb = Z48587/gi = 1107741/ug = Mm 16779/len = 621/STRA = for | 348.6 | P | 389.2 | P | 419.2 | P |
| 4 | 103341_at | Cluster Incl U49350.Mus musculus CTP synthetase mRNA, complete cds/cds = (111,1886)/gb = U49350/gi = 1515356/ | 696.8 | P | 1003.4 | P | 830.8 | P |
| | | ug = Mm 1815/len = 2681/STRA = for | | | | | | |
| 4 | 103344_at | Cluster Incl L16953: Mouse tumor cell dna-j-like protein 1 mRNA, complete cds/cds = (7,1665)/gb = L16953/gi = 473846/ | 377.3 | P | 342 | P | 292.9 | P |
| | | ug = Mm 18237/len = 1792/STRA = for | | | | | | |
| 4 | 103413_at | Cluster Incl AI021091 ub01b04 r1 Mus musculus cDNA, 5 end/clone = IMAGE-1365679/clone_end = 5/gb = AI021091/ | 221 | P | 160.7 | P | 245.4 | P |
| | | gi = 3235427/ug = Mm 18834/len = 426/STRA = for | | | | | | |
| 4 | 103415_at | Cluster Incl AI840925: UI-M-AM0-adm-g-07-0-UI.s1 Mus musculus cDNA, 3 end/clone = UI-M-AM0-adm-g-07-0-UI/ | 112.1 | P | 159.3 | P | 142 | P |
| | | clone_end = 3/gb = AI840925/gi = 5475138/ug = Mm 18847/len = 279/STRA = for | | | | | | |
| 4 | 103619_at | Cluster Incl AI850017: UI-M-BG0-aib-g-03-0-UI.s1 Mus musculus cDNA, 3 end/clone = UI-M-BG0-aib-g-03-0-UI/ | 255.4 | P | 498 | P | 539.5 | P |
| | | clone_end = 3/gb = AI850017/gi = 5493923/ug = Mm 20242/len = 428/STRA = rev | | | | | | |
| 4 | 104317_at | Cluster Incl AW123099: UI-M-BH2 1-apc-e-04-0-UI s1 Mus musculus cDNA, 3 end/clone = UI-M-BH2 1-apc-e-04-0-UI/ | 230.9 | P | 290.4 | P | 228.5 | P |
| | | clone_end = 3/gb = AW123099/gi = 6098629/ug = Mm 22417/len = 610/STRA = for | | | | | | |
| 4 | 94418_at | Cluster Incl AI839004: UI-M-AN0-acm-c-04-0-UI.s1 Mus musculus cDNA, 3 end/clone = UI-M-AN0-acm-c-04-0-UI/ | 152.2 | P | 404.5 | P | 204.7 | P |
| | | clone_end = 3/gb = AI839004/gi = 5473217/ug = Mm 26171/len = 428/STRA = for | | | | | | |
| 4 | 94970_at | Cluster Incl AI852314 UI-M-BH0-aje-g-07-0-UI.s1 Mus musculus cDNA, 3 end/clone = UI-M-BH0-aje-g-07-0-UI/ | 97.2 | P | 152.5 | P | 155.6 | P |
| | | clone_end = 3/gb = AI852314/gi = 5496220/ug = Mm 27223/len = 401/STRA = for | | | | | | |
| 4 | 95002_at | Cluster Incl AI838731 UI-M-AO0-acf-c-07-0-UI.s1 Mus musculus cDNA, 3 end/clone = UI-M-AO0-acf-c-07-0-UI/ | 520.2 | P | 684.5 | P | 648.7 | P |
| | | clone_end = 3/gb = AI838731/gi = 5472944/ug = Mm 27362/len = 328/STRA = for | | | | | | |
| 4 | 95526_at | Cluster Incl AI606243 vo02e01 x1 Mus musculus cDNA, 3 end/clone = IMAGE-1040280/clone_end = 3/gb = AI606243/ | 357.5 | P | 351.8 | P | 399.2 | P |
| | | gi = 4615410/ug = Mm 27607/len = 356/STRA = for | | | | | | |
| 4 | 95619_at | Cluster Incl AI851723 UI-M-BH0-aio-f-07-0-UI s1 Mus musculus cDNA, 3 end/clone = UI-M-BH0-aio-f-07-0-UI/ | 130 | P | 138.2 | P | 97.7 | P |
| | | clone_end = 3/gb = AI851723/gi = 5495624/ug = Mm 28037/len = 391/STRA = for | | | | | | |
| 4 | 96756_at | Cluster Incl AA693236: vr58d08 r1 Mus musculus cDNA, 5 end/clone = IMAGE-1124847/clone_end = 5/gb = AA693236/ | 152.2 | P | 122.5 | P | 115.2 | P |
| | | gi = 2694174/ug = Mm 28983/len = 409/STRA = for | | | | | | |
| 4 | 97918_at | Cluster Incl AA623587 vn73d05 r1 Mus musculus cDNA, 5 end/clone = IMAGE-1037577/clone_end = 5/gb = AA623587/ | 568.9 | P | 469.7 | P | 483.2 | P |
| | | gi = 2527483/ug = Mm.30122/len = 163/STRA = for | | | | | | |
| 4 | 97954_at | Cluster Incl X59513 Mouse 5 end of TRP1 gene for tyrosinase-related protein-1/cds = (181,183)/gb = X59513/gi = 54913/ | −37.7 | P | 193.6 | P | −15 | P |
| | | ug = Mm 30438/len = 272/STRA = for | | | | | | |
| 4 | 98024_at | Cluster Incl X55316 Nuclear transcription factor-Y beta/cds = (209,832)/gb = X55316/gl = 53362/ug = Mm 3259/len = 1234/ | 122.1 | P | 337.9 | P | 347.6 | P |
| | | STRA = for | | | | | | |
| 4 | 98404_at | Cluster Incl X64587 M. musculus mRNA for splicing factor U2AF (65 kD)/cds = UNKNOWN/gb = X64587/gi = 55100/ | 2129.3 | P | 1988.5 | P | 2150.9 | P |
| | | ug = Mm 3358/len = 2119/STRA = for | | | | | | |
| 4 | 98415_at | Cluster Incl AF046060 Mus musculus cytokine receptor related protein 4 (Cytor4) mRNA, complete cds/cds = (54,1382)/ | 500.7 | P | 430.9 | P | 432.7 | P |
| | | gb = AF046060/gi = 4105473/ug = Mm.33962/len = 2365/STRA = for | | | | | | |
| 4 | 98997_f _at | Cluster Incl AF020790 Mus musculus hair keratin acidic 5 (Ha5) mRNA, complete cds/cds = (129,1406)/gb = AF020790/ | 595.9 | P | 1332.6 | P | 886.4 | P |
| | | gi = 4103157/ug = Mm.37952/len = 1737/STRA = rev | | | | | | |
| 4 | 99452_at | Cluster Incl U49507: Mus musculus B6CBA Lisch7 mRNA, partial cds/cds = (0,1971)/gb = U49507/gi = 1236082/ | 226.9 | P | 405.9 | P | 303.9 | P |
| | | ug = Mm 4067/len = 2059/STRA = for | | | | | | |
| 4 | 100886_f_at | Cluster Incl AI841415 UI-M-AH0-acx-c-02-0-UI s1 Mus musculus cDNA, 3 end/clone = UI-M-AH0-acx-c-02-0-UI/ | 310.8 | P | 301.3 | P | 407.2 | P |
| | | clone_end = 3/gb = AI841415/gi = 5475628/ug = Mm.5381/len = 303/STRA = for | | | | | | |
| 4 | 100924_at | Cluster Incl X55123 GATA-binding protein 3/cds = (207,1538)/gb = X55123/gi = 51052/ug = Mm 606/len = 2056/STRA = for | 1211.9 | P | 2048.1 | P | 1556.1 | P |
| 4 | 101469_at | Cluster Incl AF009368 Mus musculus neural precursor cell expressed developmentally downregulated Nedd9 (Nedd9) | 164.3 | P | 200.9 | P | 151.2 | P |
| | | mRNA, complete cds/cds = (292,2793)/gb = AF009366/gi = 2454522/ug = Mm 8315/len = 2889/STRA = for | | | | | | |
| 4 | 101929_at | Cluster Incl AI836322 UI-N-AQ0-aag-a-02-0-UI s2 Mus musculus cDNA, 3 end/clone = UI-M-AQ0-aag-a-02-0-UI/ | 154.5 | P | 432.5 | P | 203.8 | P |
| | | clone_end = 3/gb = AI836322/gi = 5470530/ug = Mm.939/len = 211/STRA = for | | | | | | |
| 4 | 102280_at | Cluster Incl AB006758: Mus musculus Pcdh7 mRNA for BH-protocadherin-a, complete cds/cds = (1004,4213)/ | 124.7 | P | 276.2 | P | 210.9 | P |
| | | gb = AB006758/gi = 3513311/ug = Mm 11964/len = 5520/STRA = for | | | | | | |
| 4 | 102863_at | Cluster Incl AI550530: vx21g12.x1 Mus musculus cDNA, 3 end/clone = IMAGE-1265158/clone_end = 3/gb = AI550530/ | 282.8 | P | 234.6 | P | 229 | P |
| | | gi = 4482893/ug = Mm 1468/len = 234/STRA = for | | | | | | |
| 4 | 103243_at | Cluster Incl AF083876 Mus musculus XMP (Xmp) mRNA, complete cds/cds = (109,627)/gb = AF083876/gi = 3462841/ | 1230.7 | P | 1117.6 | P | 1079.9 | P |
| | | ug = Mm 20423/len = 747/STRA = for | | | | | | |
| 4 | 103501_at | Cluster Incl U02098 Mus musculus Pur-alpha mRNA, complete cds/cds = (518,1483)/gb = U02098/gi = 404649/ug = Mm 2236/ | 287.5 | P | 600.7 | P | 511.8 | P |
| | | len = 1609/STRA = for | | | | | | |
| 4 | 103762_at | Cluster Incl AI853340 UI-M-BH0-air-b-06-0-UI s1 Mus musculus cDNA, 3 end/clone = UI-M-BH0-air-b-06-0-UI/ | 462.2 | P | 448.5 | P | 434.4 | P |
| | | clone_end = 3/gb = AI853340/gi = 5497246/ug = Mm.24632/len = 461/STRA = for | | | | | | |
| 4 | 104183_at | Cluster Incl AI529005.ud44a03 y1 Mus musculus cDNA, 5 end/clone = IMAGE-1448716/clone_end = 5/gb-AI529005/ | 115.2 | P | 192.5 | P | 178 | P |
| | | gi = 4443140/ug = Mm 27380/len = 460/STRA = for | | | | | | |
| 4 | 104220_at | Cluster Incl AF010133 Mus musculus Smad6 mRNA, complete cds/cds = (0,1487)/gb = AF010133/gi = 2507639/ | 335.1 | P | 417.3 | P | 288.7 | P |
| | | ug = Mm.27935/len = 1488/STRA = for | | | | | | |
| 4 | 92409_at | Cluster Incl D45210: Mouse mRNA for zinc finger protein, complete cds/cds = (590,1813)/gb = D45210/gi = 633989/ | 90.9 | P | 125.2 | P | 132.8 | P |
| | | ug = Mm.4106/len = 2849/STRA = for | | | | | | |
| 4 | 92460_at | Cluster Incl AB022316: Mus musculus gene for semaphorin w/cds = (78,2411)/gb = AB022316/gi = 4589501/ug = Mm.42035/ | 1368 | P | 1109.5 | P | 1598.2 | P |
| | | len = 4024/STRA = for | | | | | | |
| 4 | 92888_s_at | Cluster Incl U34973: Mus musculus protein tyrosine phosphatase-like mRNA, unspliced c-terminal product and spliced c- | 232 | P | 291.4 | P | 344 | P |
| | | terminal and STYX, complete cds/cds = (255,872)/gb = U34973/gi = 1063624/ug = Mm 4755/len = 1254/STRA = for | | | | | | |
| 4 | 92893_at | Cluster Incl D90173 Mouse mRNA for NFI-B protein, clone NFI-B2/cds = (111,1709)/gb = D90173/gi = 220518/ug = Mm 4771/ | 243.8 | P | 205.9 | P | 209.1 | P |
| | | len = 3296/STRA = for | | | | | | |
| 4 | 93246_at | Cluster Incl AW260482: um80e10.x1 Mus musculus cDNA, 3 end/clone = IMAGE-2317674/clone_end = 3/gb = AW260482/ | 272.8 | P | 501.7 | P | 419.7 | P |
| | | gi = 6633463/ug = Mm 6043/len = 635/STRA = rev | | | | | | |
| 4 | 93439_f_at | Cluster Incl AA260005 va87f08.r1 Mus musculus cDNA, 5 end/clone = IMAGE-746435/clone_end = 5/gb = AA260005/ | 239.2 | P | 376.4 | P | 354.4 | P |
| | | gi = 1896491/ug = Mm.6617/len = 271/STRA = for | | | | | | |
| 4 | 93667_at | Cluster Incl AW120511 UI-M-BH1-ana-a-04-0-UI s2 Mus musculus cDNA, 3 end/clone = UI-M-BH1-ana-a-04-0-UI/ | −17.1 | P | 196.3 | P | 228.4 | P |
| | | clone_end = 3/gb = AW120511/gi = 6095844/ug = Mm.80527/len = 454/STRA = for | | | | | | |
| 4 | 97767_at | Cluster Incl X75649 M musculus mRNA for mouse hair acidic 2/cds = (28,1251)/gb = X75649/gi = 498977/ug = Mm 4161/ | 968.8 | P | 1491.4 | P | 1051.6 | P |
| | | len = 1591/STRA = for | | | | | | |
| 4 | 99328_at | Cluster Incl U79738 Mus musculus homeodomain protein Dix-3 mRNA, parital cds/cds = (0,695)/gb = U79738/gi = 1718494/ | 467.3 | P | 782.9 | P | 676 | P |
| | | ug = Mm.5194/len = 2221/STRA = for | | | | | | |
| 4 | 100323_at | Cluster Incl Z23077 S-adenosylmethionine decarboxylase 3/cds = (0,1004)/gb = Z23077/gi = 396140/ug = Mm 87749/ | 368.6 | P | 623.2 | P | 563.8 | P |
| | | len = 1005/STRA = for | | | | | | |
| 4 | 100324_g_at | Cluster Incl Z23077 S-adenosylmethionine decarboxylase 3/cds = (0,1004)/gb = Z23077/gi = 396140/ug = Mm 87749/ | 93.7 | P | 170.2 | P | 151.6 | P |
| | | len = 1005/STRA = for | | | | | | |
| 4 | 101254_at | Cluster Incl L32751 Lipopolysacchande response/cds = (25,675)/gb = L32751/gi = 727168/ug = Mm 7521/len = 994/STRA = for | 5097.5 | P | 5167.2 | P | 5631.1 | P |
| 4 | 102099_f_at | Cluster Incl AI843637.UI-M-AO1-aen-g-04-0-UI s1 Mus musculus cDNA, 3 end/clone = UI-M-AO1-aen-g-04-0-UI/ | 576.9 | P | 470.5 | P | 504.8 | P |
| | | clone_end = 3/gb = AI843637/gi = 5477850/ug = Mm 42435/len = 238/STRA = rev | | | | | | |
| 4 | 102103_f_at | Cluster Incl AI482454 vg40d04 x1 Mus musculus cDNA, 3 end/clone = IMAGE-863815/clone_end = 3/gb = AI482454/ | 1744.6 | P | 1533.4 | P | 1832.2 | P |
| | | gi = 4375680/ug = Mm 42484/len = 454/STRA = rev | | | | | | |
| 4 | 92614_at | Cluster Incl M60523.Inhibitor of DNA binding 3/cds = (57,416)/gb = M60523/gi = 193872/ug = Mm 110/len = 969/STRA = for | 351.1 | P | 259.2 | P | 335 | P |
| 4 | 93348_at | Cluster Incl AA760359 vv76a05 r1 Mus musculus cDNA, 5 end/clone = IMAGE-1228304/clone_end = 5/gb = AA760359/ | 169.2 | P | 104.3 | M | 178 | P |
| | | gi = 2808153/ug = Mm 18803/len = 373/STRA = for | | | | | | |
| 4 | 97814_at | Cluster Incl x75650 M musculus mRNA for mouse hair acidic 3/cds = (0,971)/gb = X75650/gi = 498975/ug = Mm.28178/ | 289.6 | P | 785 | P | 344.9 | P |
| | | len = 1304/STRA = for | | | | | | |
| 4 | 95649_at | Cluster Incl AI844440 UI-M-AJ1-ahe-a-02-0-UI s1 Mus musculus cDNA, 3 end/clone = UI-M-AJ1-ahe-a-02-0-UI/ | 341 | P | 435.1 | P | 479 | P |
| | | clone_end = 3/gb = AI844440/gi = 5488346/ug = Mm 29503/len = 301/STRA = for | | | | | | |
| 4 | 95677_at | Cluster Incl AA881621 vx20h04 r1 Mus musculus cDNA, 5 end/clone = IMAGE-1265047/clone_end = 5/gb = AA881621/ | 201.6 | P | 131.6 | P | 272 | P |
| | | gi = 2990931/ug = Mm 29606/len = 507/STRA = for | | | | | | |
| 4 | 99644_at | Cluster Incl AI845855-UI-M-AO1-aeg-e-11-0-UI s1 Mus musculus cDNA, 3 end/clone = UI-M-AO1-aeg-e-11-0-UI/ | 659.9 | P | 428.6 | M | 725.5 | P |
| | | clone_end = 3/gb = AI845855/gi = 5489761/ug = Mm 43636/len = 407/STRA = for | | | | | | |
| 4 | 103558_at | Cluster Incl M27734 Mouse acedic keratin type I mRNA, complete cds/cds = (69,1319)/gb = M27734/gi = 198585/ | 143.4 | A | 825.7 | P | 268.6 | P |
| | | ug = Mm.19109/len = 1585/STRA = for | | | | | | |
| 4 | 103803_at | Cluster Incl AB013849 Mus musculus mRNA for peptidylarginine deiminase type III, complete cds/cds = (42,2038)/ | 137.5 | P | 272.5 | P | 161.1 | P |
| | | gb = AB013849/gi = 3645569/ug = Mm.20851/len = 3068/STRA = for | | | | | | |
| 4 | 104124_at | Cluster Incl AW123135 UI-M-BH2.1-apd-a-07-0-UI s1 Mus musculus cDNA, 3 end/clone = UI-M-BH2-apd-a-07-0-UI/ | 79.6 | P | 170.4 | P | 106.1 | P |
| | | clone_end = 3/gb = AW123135/gi = 6098665/ug = Mm 22028/len = 503/STRA = for | | | | | | |
| 4 | 96138_at | Cluster Incl AA682129: vu13b07.r1 Mus musculus cDNA, 5 end/clone = IMAGE-1180501/clone_end = 5/gb = AA682129/ | 169 | P | 373.9 | P | 332.5 | P |
| | | gi = 2663161/ug = Mm 28173/len = 611/STRA = for | | | | | | |
| 4 | 96822_at | Cluster Incl AA734426 vr90e10 r1 Mus musculus cDNA, 5 end/clone = IMAGE-1136010/clone_end = 5/gb = AA734428/ | 480.4 | P | 428.8 | A | 449.3 | P |
| | | gi = 2756095/ug = Mm 29383/len = 409/STRA = rev | | | | | | |
| 4 | 97983_s_at | Cluster Incl D45903. Mus musculus mRNA for unc-18 homologue, complete cds/cds = (35,1819)/gb = D45903/gi = 1944321/ | 156.7 | A | 214.4 | P | 190.6 | P |
| | | ug = Mm.3129/len = 3412/STRA = for | | | | | | |
| 4 | 99502_at | Cluster Incl U80078 Mus musculus transcription factor BFCOL1 mRNA, complete cds/cds = (86,2395)/gb = U80078/ | 571.9 | P | 584.3 | P | 863.2 | P |
| | | gi = 1724123/ug = Mm 417/len = 2426/STRA = for | | | | | | |
| 4 | 100895_at | Cluster Incl AW049497: UI-M-BH1-anf-a-01-0-UI s1 Mus musculus cDNA, 3 end/clone = UI-M-BH1-anf-a-01-0-UI/ | 50.7 | P | 57.5 | P | 68.1 | P |
| | | clone_end = 3/gb = AW049497/gi = 5910026/ug = Mm 567/len = 291/STRA = for | | | | | | |
| 4 | 101376_at | Cluster Incl D86419 Mus musculus mRNA for glycine tyrosine-rich hair keratin protein, complete cds/cds = (2,235)/ | 230.2 | A | 751.8 | P | 467.2 | P |
| | | gb = D86419/gi = 1944392/ug = Mm 6973/len = 520/STRA = for | | | | | | |
| 4 | 103791_at | Cluster Incl AW048763 UI-M-BH1-amy-h-02-0-UI s1 Mus musculus cDNA, 3 end/clone = UI-M-BH1-amy-h-02-0-UI/ | 86.7 | P | 98.5 | P | 39.1 | P |
| | | clone_end = 3/gb = AW048763/gi = 5909292/ug = Mm 24958/len = 589/STRA = for | | | | | | |
| 4 | 103998_at | Cluster Incl AW122699 UI-M-BH2 2-aot-b-09-0-UI s1 Mus musculus cDNA, 3 end/clone = UI-M-BH2 2-aot-b-09-0-UI/ | 55.5 | P | 95.2 | P | 34.2 | A |
| | | clone_end = 3/gb = AW122699/gi = 6098229/ug = Mm 26532/len = 435/STRA = for | | | | | | |
| 4 | 104242_f_at | Cluster Incl AI835622 UI-M-AI0-aap-f-12-0-UI s1 Mus musculus cDNA, 3 end/clone = UI-M-AI0-aap-f-12-0-UI/clone_end = 3/ | 1003.9 | P | 1507.2 | P | 1290.7 | P |
| | | gb = AI835622/gi = 5469835/ug = Mm.2821/len = 344/STRA = for | | | | | | |
| 4 | 104643_at | Cluster Incl AI850848 UI-M-BH0-ajt-b-11-0-UI s1 Mus musculus cDNA, 3 end/clone = UI-M-BH0-ajt-b-11-0-UI/clone_end = 3/ | 262.8 | P | 405.4 | P | 326 | P |
| | | gb = AI850846/gi = 5494752/ug = Mm 31267/len = 341/STRA = for | | | | | | |
| 4 | 104688_at | Cluster Incl Y09010 M musculus mRNA for serine/threonine kinase/cds = (138,2621)/gb = Y09010/gi = 1654011/ | 144.7 | P | 128.7 | P | 119.7 | P |
| | | ug = Mm 3313/len = 2732/STRA = for | | | | | | |
| 4 | 104696_at | Cluster Incl AJ009840 Mus musculus cathepsin E gene, exon 1, partial/cds = (0,1193)/gb = AJ009840/gi = 3392946/ | 15.5 | M | 203.8 | P | 92.9 | P |
| | | ug = Mm 33671/len = 1194/STRA = for | | | | | | |
| 4 | 93720_at | Cluster Incl AB005623: Mus musculus mRNA for 1-acyl-sn-glycerol-3-phosphate acyltransferase, complete cds/ | 41 | P | 69.2 | P | 179.2 | P |
| | | cds = (318,1175)/gb = AB005623/gi = 2457309/ug = Mm.8684/len = 1256/STRA = for | | | | | | |
| 4 | 93935_at | Cluster Incl AF036332: Mus musculus nemo-like kinase (NIk) mRNA, complete cds/cds = (322,1869)/gb = AF036332/ | 40 | P | 52.5 | P | 63.3 | P |
| | | gi = 2828007/ug = Mm.9001/len = 2603/STRA = for | | | | | | |
| 4 | 98340_at | Cluster Incl AA407332: EST00652 Mus musculus cDNA, 5 end/clone = C0001A09/clone_end = 5/gb = AA407332/ | 28.9 | P | 40.8 | P | 69.1 | A |
| | | gi = 2065567/ug = Mm 43174/len = 365/STRA = rev | | | | | | |
| 4 | 100381_at | Cluster Incl M12347: Actin, alpha 1, skeletal muscle/cds = (70,1203)/gb = M1234/gi = 191572/ug = Mm.89137/len = 1446/ | 2870.5 | P | 3303.2 | P | 3452.1 | P |
| | | STRA = for | | | | | | |
| 4 | AFFX-BioB- | E Coli BioB 3 prime | 250.5 | M | 423.8 | P | 411 | P |
| | 3_at | | | | | | | |
| 4 | 93046_at | Cluster Incl AW045233 UI-M-BH1-alh-f-10-0-UI s1 Mus musculus cDNA, 3 end/clone = UI-M-BH1-alh-f-10-0-UI/ | 71 | A | 59 | P | 127.9 | P |
| | | clone_end = 3/gb = AW045233/gi = 5905762/ug = Mm 1521/len = 412/STRA = for | | | | | | |
| 4 | 93971_f_at | Cluster Incl AI838669 UI-M-AO0-aca-e-04-0-UI s1 Mus musculus cDNA, 3 end/clone = UI-M-AO0-aca-e-04-0-UI/ | 394.6 | A | 348.4 | P | 352.2 | A |
| | | clone_end = 3/gb = AI838669/gi = 5472882/ug = Mm 21667/len = 452/STRA = for | | | | | | |
| 4 | 94064_at | Cluster Incl U05343: Mus musculus Balb/c zinc finger protein PZF (Pzf) mRNA, complete cds/cds = (137,1504)/gb = U05343/ | 303.8 | P | 393.2 | P | 413 | P |
| | | gi = 453375/ug = Mm 22066/len = 1514/STRA = for | | | | | | |
| 4 | 94804_at | Cluster Incl L27453: Pre B-cell leukemia transcription factor 1/cds = (44,1087)/gb = L27453/gi = 456108/ug = Mm 22675/ | 193 | P | 154 | P | 34.8 | P |
| | | len = 1634/STRA = for | | | | | | |
| 4 | 94493_at | Cluster Incl AF095905: Mus musculus CPETR2 (Cpetr2) mRNA, complete cds/cds = (55,714)/gb = AF095905/gi = 4262394/ | 126.5 | P | 187.4 | P | 142.9 | P |
| | | ug = Mm 28921/len = 1090/STRA = for | | | | | | |
| 4 | 95658_at | Cluster Incl AW061085 UI-M-BH1-anp-c-04-0-UI s1 Mus musculus cDNA, 3 end/clone = UI-M-BH1-anp-c-04-0-UI/ | 90.9 | P | 73.7 | P | 84.1 | P |
| | | clone_end = 3/gb = AW061085/gi = 6008836/ug = Mm 29542/len = 464/STRA = for | | | | | | |
| 4 | 96918_at | Cluster Incl AI790931 uk29g y1 Mus musculus cDNA, 5 end/clone = IMAGE-1970450/clone_end = 5/gb = AI790931/ | 51.5 | A | 573.9 | P | 195.1 | A |
| | | gi = 5338647/ug = Mm 30154/len = 660/STRA = for | | | | | | |
| 4 | 102415_r_at | Cluster Incl U28423 Mus musculus protein kinease inhibitor p58 mRNA, complete cds/cds = (100,1614)/gb = U28423/ | 154.3 | P | 218.9 | P | 323.9 | P |
| | | gi = 1353271/ug = Mm 12616/len = 1615/STRA = for | | | | | | |
| 4 | 104096_at | Cluster Incl Y16386: Mus musculus mRNA for origin recognition complex subunit 4/cds = (0,1301)/gb = Y16386/gi = 3288543/ | 63.2 | A | 118.8 | P | 65.2 | P |
| | | ug = Mm 21834/len = 1302/STRA = for | | | | | | |
| 4 | 104626_at | Cluster Incl AI787137 ui85c11.y1 Mus musculus cDNA, 5 end/clone = IMAGE-1889204/clone_end = 5/gb = AI787137/ | 111.8 | A | 132.6 | P | 172.1 | P |
| | | gi = 5334853/ug = Mm 24277/len = 297/STRA = for | | | | | | |
| 4 | 96177_at | Cluster Incl AI854590 UI-M-BH0-ake-b-04-0-UI s1 Mus musculus cDNA, 3 end/clone = UI-M-BH0-ake-b-04-0-UI/ | −133.8 | P | −126.1 | P | 151.2 | P |
| | | clone_end = 3/gb = AI854590/gi = 549896/ug = Mm.28412/len = 435/STRA = for | | | | | | |
| 4 | 98407_at | Cluster Incl U07602 Ephnn B1/cds = (1643,2680)/gb = U07602/gi = 2182217/ug = Mm 3374/len = 2692/STRA = rev | 214.6 | P | 205.5 | P | 301.7 | P |
| 4 | 102308_at | Cluster Incl AF045582: Mus musculus tubby protein 3 (Tulp3) mRNA, complete cds/cds = (26,1408)/gb = AF045582/ | 447.6 | P | 433.1 | P | 486.5 | P |
| | | gi = 3372490/ug = Mm 12887/len = 1508/STRA = for | | | | | | |
| 4 | 103552_at | Cluster Incl AW046470 UI-M-BH1-akp-h-12-0-UI s1 Mus musculus cDNA, 3 end/clone = UI-M-BH1-akp-h-12-0-UI/ | 128.2 | P | 108.3 | P | 108.8 | P |
| | | clone_end = 3/gb = AW046470/gi = 5906999/ug = Mm 23822/len = 449/STRA = rev | | | | | | |
| 4 | 95801_s_at | Cluster Incl L36316: Zinc finger protein 63/cds = (0,556)/gb = L36316/gi = 538414/ug = Mm 87838/len = 858/STRA = for | 108.8 | P | 144.6 | P | 129.3 | P |
| 4 | 93981_at | Cluster Incl J03520 Plasminogen acitvator, tissue/cds = (93,1772)/gb = J03520/gi = 202109/ug = Mm.21702/len = 2519/ | 244.5 | A | 434.8 | P | 622.1 | P |
| | | STRA = for | | | | | | |
| 4 | 94222_at | Cluster Incl AI838737 UI-M-AO0-acf-d-01-0-UI.s1 Mus musculus cDNA, 3 end/clone = UI-M-AO0-acf-d-01-0-UI/ | 527.7 | A | 637.8 | P | 688.5 | P |
| | | clone_end = 3/gb = AI838737/gi = 5472950/ug = Mm 22248/len = 399/STRA = for | | | | | | |
| 4 | 94916_at | Cluster Incl AW122260 UI-M-BH2 2-aov-g-11-0-UI s1 Mus musculus cDNA, 3 end/clone = UI-M-BH2 2-aov-g-11-0-UI/ | 173.6 | A | 394.9 | P | 286.4 | A |
| | | clone_end = 3/gb = AW122260/gi = 6097729/ug = Mm 24155/len = 161/STRA = for | | | | | | |
| 4 | 96294_s_at | Cluster Incl AI854235 UI-M-BH0-ajz-g-07-0-UI s1 Mus musculus cDNA, 3 end/clone = UI-M-BH0-ajz-g-07-0-UI/ | 61.8 | M | 95.5 | M | 166.9 | P |
| | | clone_end = 3/gb = AI854235/gi = 5498141/ug = Mm 29898/len = 519/STRA = for | | | | | | |
| 4 | 100607_at | Cluster Incl AF026124: Mus musculus schwannoma-associated protein (SAM9) mRNA, complete cds/cds = (417,1883)/ | 440.8 | P | 362.1 | A | 415.8 | P |
| | | gb = AF026124/gi = 2565395/ug = Mm 6483/len = 2248/STRA = for | | | | | | |
| 4 | 102826_at | Cluster Incl J05663: Androgen regulated vas deferens protein/cds = (20,970)/gb = J05663/gi = 199897/ug = Mm.14460/ | 74 | P | 99.4 | M | 106.7 | P |
| | | len = 1225/STRA = for | | | | | | |
| 4 | 103006_at | Cluster Incl AB012276 Mus musculus MRNA for ATFx, partial cds/cds = (0,254)/gb = AB012276/gi = 2988372/ug = Mm 1566/ | 255.2 | A | 365.8 | A | 371.1 | P |
| | | len = 850/STRA = for | | | | | | |
| 4 | 103565_at | Cluster Incl AI843358 UI-M-AQ1-adz-d-04-0-UI s1 Mus musculus cDNA, 3 end/clone = UI-M-AQ1-adz-d-04-0-UI/ | 388.4 | P | 356.7 | M | 358.1 | P |
| | | clone_end = 3/gb = AI84358/gi = 5477571/ug = Mm.1915/len = 444/STRA = for | | | | | | |
| 4 | 104346_at | Cluster Incl AI841914 UI-M-AO0-acd-f-08-0-UI.s1 Mus musculus cDNA, 3 end/clone = UI-M-AO0-acd-f-08-0-UI/ | 163.6 | M | 258.9 | M | 213.3 | P |
| | | clone_end = 3/gb = AI841914/gi = 5476127/ug = Mm.22537/len = 421/STRA = for | | | | | | |
| 4 | 95596_at | Cluster Incl AW122957 UI-M-BH2 1-aoz-e-12-0-UI.s2 Mus musculus cDNA, 3 end/clone = UI-M-BH2 1-aoz-e-12-0-UI/ | 144.7 | A | 127.3 | P | 173.4 | P |
| | | clone_end = 3/gb = AW122957/gi = 6098487/ug = Mm.27918/len = 443/STRA = for | | | | | | |
| 4 | 96735_at | Cluster Incl AW049732 UI-M-BH1-ani-g-05-0-UI.s1 Mus musculus cDNA, 3 end/clone = UI-M-BH1-ani-g-05-0-UI/ | 172.5 | A | 259.7 | P | 463.7 | P |
| | | clone_end = 3/gb = AW049732/gi = 5910261/ug = Mm.28896/len = 372/STRA = for | | | | | | |
| 4 | 98998_r_at | Cluster Incl AF020790 Mus musculus hair keratin acidic 5 (Ha5) mRNA, complete cds/cds = (129,1406)/gb = AF020790/ | 42.7 | A | 418.5 | P | 68.5 | A |
| | | gi = 4103157/ug = Mm.37952/len = 1737/STRA = rev | | | | | | |
| 4 | 102642_at | Cluster Incl L33415 Natural resistance-associated macrophage protein 2/cds = (60,1766)/gb = L33415/gi = 755040/ | 773.8 | A | 1368 | P | 1094.4 | P |
| | | ug = Mm.1304/len = 2972/STRA = for | | | | | | |
| 4 | 103257_at | Cluster Incl AA690483: vu52f06.r1 Mus musculus cDNA, 5 end/clone = IMAGE-1195043/clone_end = 5/gb = AA690483/ | 61.6 | A | 154.9 | P | 70.3 | A |
| | | gl = 2691419/ug = Mm.2073/len = 302/STRA = for | | | | | | |
| 4 | 103965_at | Cluster Incl AI837032 UI-M-AP0-abj-d-02-0-UI s2 Mus musculus cDNA, 3 end/clone = UI-M-AP0-abj-d-02-0-UI/ | 266.6 | A | 133.7 | P | 524.2 | P |
| | | clone_end = 3/gb = AI837032/gi = 5471245/ug = Mm.25640/len = 289/STRA = for | | | | | | |
| 4 | 98314_g_at | Cluster Incl AB025413.Mus musculus mRNA for Ten-m4, complete cds/cds = (187,8502)/gb = AB025413/gi = 4760781/ | 250.7 | P | 217.6 | P | 178.4 | A |
| | | ug = Mm.42204/len = 8585/STRA = for | | | | | | |
| 4 | 100673_f_at | Cluster Incl U10091 Killer cell lectin-like receptor, subfamily A, member 5/cds = (108,908)/gb = U10091/gi = 533489/ | −3.6 | A | 33.5 | A | 18.7 | P |
| | | ug = Mm.14111/len = 1157/STRA = for | | | | | | |
| 4 | 98881_at | Cluster Incl AI853675: UI-M-BH0-ajq-g-08-0-UI.s1 Mus musculus cDNA, 3 end/clone = UI-M-BH0-ajq-g-08-0-UI/ | 140.5 | A | 144.1 | A | 120.6 | P |
| | | clone_end = 3/gb = AI853675/gi = 5497621/ug = Mm.28525/len = 528/STRA = for | | | | | | |
| 4 | 101006_at | Cluster Incl M35797: T-complex protein 1, related sequence 1/cds = (0,852)/gb = M35797/gi = 975268/ug = Mm.6797/ | 318.3 | A | 468.7 | A | 359.2 | A |
| | | len = 1692/STRA = for | | | | | | |
| 4 | 102989_at | Cluster Incl U07808: Metallothionein 4/cds = (21,209)/gb = U07808/gi = 466268/ug = Mm 1536/len = 372/STRA = for | −40.9 | A | 51.3 | P | −11.5 | A |
| 4 | 103805_at | Cluster Incl AF092840: Mus musculus nibnn mRNA, complete cds/cds = (90,2345)/gb = AF092840/gi = 3676834/ | 154.6 | A | 175.5 | A | 159.1 | P |
| | | ug = Mm 20866/len = 2473/STRA = for | | | | | | |
| 4 | 104364_at | Cluster Incl AF039840 Mus musculus mitogen-activated protein kinase-activated protein kinase MRNA, complete cds/ | −18.2 | A | −18.3 | P | 70.6 | A |
| | | cds = (706,2127)/gb = AF039840/gi = 2911812/ug = Mm 22612/len = 2243/STRA = for | | | | | | |
| 4 | 95556_at | Cluster Incl AI841364: UI-M-AM0-adv-f-01-0-UI s1 Mus musculus cDNA, 3 end/clone = UI-M-AM0-adv-f-01-0-UI/ | −28.7 | P | −176.5 | A | 125.2 | P |
| | | clone_end = 3/gb = AI841364/gi = 5475577/ug = Mm 27754/len = 389/STRA = for | | | | | | |
| 4 | 97343_at | Cluster Incl AI852735 UI-M-BH0-aji-h-03-0-UI s1 Mus musculus cDNA, 3 end/clone = UI-M-BH0-aji-h-03-0-UI/clone_end = 3/ | 235.3 | A | 465.4 | P | 836.4 | P |
| | | gb = AI852735/gi = 5496641/ug = Mm 29604/len = 292/STRA = rev | | | | | | |
| 4 | 98429_at | Cluster Incl AB009653: Mus musculus mRNA for lysophospholipase II, complete cds/cds = (105,800)/gb = AB009653/ | 440.4 | A | 566.7 | M | 576.2 | P |
| | | gi = 4589452/ug = Mm 34302/len = 1593/STRA = for | | | | | | |
| 4 | 98488_at | Cluster Incl AJ223361: Mus musculus mRNA for myosin heavy chain 28, partial/cds = (0,93)/gb = AJ223361/gi = 4582681/ | 24.3 | A | 96.5 | P | 69.4 | P |
| | | ug = Mm 35531/len = 199/STRA = for | | | | | | |
| 4 | 101869_s_at | Cluster Incl J00413 Hemoglobin, beta adult major chain/cds = (0,443)/gb = J00413/gi = 193793/ug = Mm 87778/len = 444/ | 20.4 | A | 5113.9 | P | 3616.8 | P |
| | | STRA = for | | | | | | |
| 4 | 103449_at | Cluster Incl AA795923 vu05110 r1 Mus musculus cDNA, 5 end/clone = IMAGE-1179787/clone_end = 5/gb = AA795923/ | 262.6 | P | 323.4 | A | 328.6 | A |
| | | gi = 2858878/ug = Mm 21577/len = 558/STRA = for | | | | | | |
| 4 | 104157_at | Cluster Incl AW125643: UI-M-BH2.2.aqi-a-09-0-UI s1 Mus musculus cDNA, 3 end/clone = UI-M-BH2 2-aqi-a-09-0-UI/ | −5.2 | A | 57 | A | 44.7 | A |
| | | clone_end = 3/gb = AW125643/gi = 6101173/ug = Mm 27090/len = 462/STRA = rev | | | | | | |
| 4 | 93901_at | Cluster Incl U78315 Pale ear/cds = (139,2250)/gb = U78315/gi = 2190591/ug = Mm 89325/len = 2687/STRA = for | 162.9 | M | 331.7 | P | 133.6 | A |
| 4 | 94781_at | Cluster Incl V00714: Hemoglobin alpha, adult chain 1/cds = (33,461)/gb = V00714/gi = 49899/ug = Mm 16820/len = 564/ | 853.6 | A | 4008.8 | P | 3580.4 | P |
| | | STRA = for | | | | | | |
| 4 | 98330_at | Cluster Incl D70849: Zinc finger protein of the crebellum 3/cds = (538,1938)/gb = D70849/gi = 1345414/ug = Mm 4265/ | 169.3 | P | 204.7 | P | 167 | P |
| | | len = 3509/STRA = for | | | | | | |
| 4 | 95858_at | Cluster Incl C76988 C76988 Mus musculus cDNA, 3 end/clone = J0023H02/clone_end = 3/gb = C76988/gi = 2517318/ | 89.8 | P | 168.3 | P | 133.5 | P |
| | | ug = Mm 24929/len = 594/STRA = rev | | | | | | |
| 4 | 93556_at | Cluster Incl X99143: M musculus mRNA for hair keratin, mHb6/cds = (0,1370)/gb = X99143/gi = 1903237/ug = Mm 20285/ | 309.2 | A | 741.9 | M | 233.5 | A |
| | | len = 1808/STRA = for | | | | | | |
| 4 | 94828_at | Cluster Incl AF004927 Opioid receptor, sigma 1/cds = (61,732)/gb = AF004927/gi = 3493173/ug = Mm 22745/len = 1618/ | 165.2 | P | 190.9 | A | 236.8 | P |
| | | STRA = for | | | | | | |
| 4 | 103833_at | Cluster Incl AF077659 Mus musculus homeodomain-interacting protein kinase 2 mRNA, complete cds/cds = (157,3726)/ | 20.7 | A | 116.3 | M | 84.4 | P |
| | | gb = AF077659/gi = 3702957/ug = Mm 20934/len = 3964/STRA = for | | | | | | |
| 4 | 98430_at | Cluster Incl X85169 Surfeit gene 5/cds = (188,610)/gb = X85169/gi = 1150511/ug = Mm 3434/len = 3424/STRA = for | 30.3 | P | −158.3 | A | 47.8 | M |
| 4 | 99521_at | Cluster Incl AB020239 Mus musculus mRNA for adenytate kinase 3, complete cds/cds = (82,753)/gb = AB020239/ | 33.3 | A | 52.7 | M | 149.1 | P |
| | | gi = 4760605/ug = Mm 42040/len = 916/STRA = for | | | | | | |
| 4 | 101863_at | Cluster Incl C78246 C78246 Mus musculus cDNA, 3 end/clone = J0044H11/clone_end = 3/gb = C78246/gi = 2518576/ | 64.7 | A | 79.6 | M | 63.4 | P |
| | | ug = Mm 87617/len = 546/STRA = rev | | | | | | |
| 4 | 102732_at | Cluster Incl AI849746: UI-M-AL1-ahn-d-09-0-UI s1 Mus musculus cDNA, 3 end/clone = UI-M-AL1-ahn-d-09-0-UI/ | 170.7 | P | 171.4 | P | 152.2 | P |
| | | clone_end = 3/gb = AI849746/gi = 5493652/ug = Mm 14458/len = 319/STRA = for | | | | | | |
| 4 | 103447_at | Cluster Incl AI835989.UI-M-AJ0-abb-a-04-0-UI s1 Mus musculus cDNA, 3 end/clone = UI-M-AJ0-abb-a-04-0-UI/ | 240.7 | A | 197.4 | P | 287.6 | P |
| | | clone_end = 3/gb = AI835989/gi = 5470242/ug = Mm 21557/len = 316/STRA = for | | | | | | |
| 4 | 93953_at | Cluster Incl D89871 Mus musculus mRNA for serine protease, complete cds/cds = (244,2529)/gb = D89871/gi = 2696702/ | 24.8 | A | 230.1 | P | 191.8 | P |
| | | ug = Mm 9431/len = 2614/STRA = for | | | | | | |
| 4 | 94712_at | Cluster Incl U73620 Vascular endothelial growth factor C/cds = (153,1400)/gb = U73620/gi = 1778356/ug = Mm 1402/ | 22 | A | −25.8 | A | 25.8 | A |
| | | len = 1818/STRA = for | | | | | | |
| 4 | 99681_at | Cluster Incl T25554: EST00587 Mus musculus cDNA, 3 end/clone = B0001C09/clone_end = 3/gb = T25554/gi = 562875/ | 127.8 | A | 125.2 | A | 137.1 | P |
| | | ug = Mm 23757/len = 275/STRA = rev | | | | | | |
| 4 | 93022_i_at | Cluster Incl M32459: Mouse histone H3 (H3 2-221) gene, complete cds/cds = (21,431)/gb = M32459/gi = 193855/ | −7.8 | A | 72.4 | P | 45.6 | A |
| | | ug = Mm 14775/len = 595/STRA = for | | | | | | |
| 4 | 93594_r_at | Cluster Incl U87948 Epithelial membrane protein 3/cds = (201,692)/gb = U87948/gi = 2358253/ug = Mm 20829/len = 695/ | 426.7 | A | 398.8 | M | 620.1 | M |
| | | STRA = for | | | | | | |
| 4 | 99080_at | Cluster Incl AI853930 UI-M-BH0-aju-g-05-0-UI s1 Mus musculus cDNA, 3 end/clone = UI-M-BH0-aju-g-05-0-UI/ | 186 | A | 253.7 | A | 298.1 | P |
| | | clone_end = 3/gb = AI853930/gi = 5497836/ug = Mm 3886/len = 442/STRA = for | | | | | | |
| 4 | 103595_at | Cluster Incl AW012359 uo03h08 y1 Mus musculus cDNA, 5 end/clone = IMAGE-2582367/clone_end = 5/gb = AW012359/ | 243.7 | A | 217.4 | A | 219.2 | P |
| | | gi = 5861137/ug = Mm 19974/len = 537/STRA = for | | | | | | |
| 4 | 104767_f_at | Cluster Incl AW060921.UI-M-BH1-anv-c-12-0-UI s1 Mus musculus cDNA, 3 end/clone = UI-M-BH1-anv-c-12-0-UI/ | 312 | A | 272.3 | A | 368.4 | A |
| | | clone_end = 3/gb = AW060921/gi = 6008672/ug = Mm 24875/len = 438/STRA = for | | | | | | |
| 4 | 96775_at | Cluster Incl X56690: Chromobox homolog (Drosophila HP1 beta)/cds = (0,557)/gb = X56690/gi = 53162/ug = Mm.29055/ | 197.6 | A | 243.1 | P | 175.9 | P |
| | | len = 558/STRA = for | | | | | | |
| 4 | 97997_at | Cluster Inci U88566 Mus musculus secreted frizzled related protein sFRP-1 (Sfrp1) mRNA, complete cds/cds = (256,1200)/ | 107.8 | M | 99.6 | P | 108.8 | P |
| | | gb = U88566/gi = 1946340/ug = Mm 3171/len = 2659/STRA = for | | | | | | |
| 4 | 102192_r_at | Cluster Incl AF068246.Mus musculus SA protein mRNA, complete cds/cds = (274,2010)/gb = AF068246/gi = 3928675/ | 11.9 | A | 31.3 | A | 9.3 | A |
| | | ug = Mm 9821/len = 2544/STRA = rev | | | | | | |
| 4 | 102652_at | Cluster Incl X56959 POU domain, class 3, transcription factor 1/cds = (35,1384)/gb = X56959/gi = 49681/ug = Mm 1330/ | 196.4 | A | 311.7 | P | 285.4 | P |
| | | len = 1516/STRA = for | | | | | | |
| 4 | 103225_at | Cluster Incl AW018420: ul95h03 x1 Mus musculus cDNA, 3 end/clone = IMAGE-21598614/clone_end = 3/gb = AW018420/ | 140.9 | A | 128.8 | A | 132.2 | A |
| | | gi = 5871950/ug = Mm 20169/len/STRA = for | | | | | | |
| 4 | 92913_at | Cluster Incl Z48670: M. musculus mRNA for ALDR/cds = (203,2428)/gb = Z48670/gi = 1107731/ug = Mm.4817/len = 2531/ | −19.3 | A | 102.3 | A | 117.4 | P |
| | | STRA = for | | | | | | |
| 4 | 93634_at | Cluster Incl AW125157: UI-M-BH2-1-apz-d-12-0-UI s1 Mus musculus cDNA, 3 end/clone = UI-M-BH2-1-apz-12-0-UI/ | 363 | M | 561.7 | A | 444.4 | P |
| | | clone_end = 3/gb = AW125157/gi = 6100687/ug = Mm 76794/len = 484/STRA = for | | | | | | |
| 4 | 93889_f_at | Cluster Incl X90778: M. musculus H2B gene/cds = (0,383)/gb = X90778/gi = 1518337/ug = Mm 89080/len = 384/STRA = rev | 57.6 | P | 49 | A | 45.7 | M |
| 4 | 95904_at | Cluster Incl AI835760 UI-M-Al0-aan-h-04-0-UI.s1 Mus musculus cDNA, 3 end/clone = UI-M-Al0-aan-h-04-0-UI/ | 301.1 | M | 439.1 | A | 245.5 | M |
| | | clone_end = 3/gb = AI835760/gi = 5469973/ug = Mm 24347/len = 490/STRA = for | | | | | | |
| 4 | 97703_at | Cluster Incl AI427631 mj85e04 x1 Mus musculus cDNA, 3 end/clone = IMAGE-482910/clone_end = 3/gb = AI427631/ | −40.5 | A | −47.8 | A | −28.4 | A |
| | | gi = 4273557/ug = Mm 38034/len = 290/STRA = rev | | | | | | |
| 5 | 92568_at | Cluster Incl M74555 House-keeping protein 1/cds = (87,1277)/gb = M74555/gi = 193870/ug = Mm 103/len = 2415/STRA = for | 116.2 | P | 226.4 | P | 226.4 | P |
| 5 | 92581_at | Cluster Incl U07159 Acetyl coenzyme A dehydrogenase, medium chain/cds = (7,1272)/gb = U07159/gi = 463908/ | 358.5 | P | 478 | P | 242.8 | P |
| | | ug = Mm 10530/len = 1846/STRA = for | | | | | | |
| 5 | 92640_at | Cluster Incl U47024: Materrial embryonic message 3/cds = (137,2401)/gb = U47024/gi = 1354049/ug = Mm 1178/len = 3128/ | 548.2 | P | 891.7 | P | 903.1 | P |
| | | STRA = for | | | | | | |
| 5 | 92773_at | Cluster Incl AF079528 Mus musculus IER5 (ler5) gene, complete cds/cds = (0,926)/gb = AF079528/gi = 3386543/ | 159.4 | P | 272.2 | P | 178.2 | P |
| | | ug = Mm 12246/len = 927/STRA = for | | | | | | |
| 5 | 92789_f_at | Cluster Incl Y12474 M.musculous mRNA for centrin gene/cds = (0,503)/gb = Y12474/gi = 2246423/ug = Mm.12481/len = 504 | 301.9 | P | 430.3 | P | 196 | P |
| | | STRA = for | | | | | | |
| 5 | 92818_at | Cluster Incl AF077330 Mus musculus NEDD8-conjugating enzyme (Uba3) mRNA, complete cds/cds = (39,1364)/ | 157.9 | P | 305.7 | P | 210 | P |
| | | gb = AF077330/gi = 3335647/ug = Mm 12892/len = 2117/STRA = for | | | | | | |
| 5 | 93025_at | Cluster Incl AI835257 UI-M-AQ0-aab-b-03-0-UI s1 Mus musculus cDNA, 3 end/clone = UI-M-AQ0-aab-b-03-0-UI/ | 1089.4 | P | 863.3 | P | 956.2 | P |
| | | clone_end = 3/gb = AI835257/gi = 5469470/ug = Mm 1479/len = 342/STRA = for | | | | | | |
| 5 | 93081_at | Cluster Incl U35142: Mus mussculus retinoblastoma-binding protein (mRbAp46) mRNA, complete cds/cds = (259,1536)/ | 1894.3 | P | 1792.2 | P | 1703.2 | P |
| | | gb = U35142/gi = 106276/ug = Mm.1603/len = 1748/STRA = for | | | | | | |
| 5 | 93082_at | Cluster Incl AW124081 UI-M-BH2 1-apn-g-11-0-UI.s1 Mus musculus cDNA, 3 end/clone = UI-M-BH2 1-apn-g-11-0-UI/ | 1608.1 | P | 1901.2 | P | 1527.3 | P |
| | | clone_end = 3/gb = AW124081/gi = 6099611/ug = Mm.1608/len = 417/STRA = for | | | | | | |
| 5 | 93088_at | Cluster Incl X01838 Beta-2 microglobulin/cds = (52,411)/gb = X01838/gi = 50104/ug = Mm 163/len = 647/STRA = for | 485.7 | P | 890.1 | P | 1326.6 | P |
| 5 | 93089_at | Cluster Incl X12507 Eukaryotic translation initiation facto 4A2/cds = (36,1259)/gb = X12507/gi = 50822/ug = Mm 16323/ | 696.6 | P | 830.9 | P | 567.5 | P |
| | | len = 1894/STRA = for | | | | | | |
| 5 | 93118_at | Cluster Incl AI844131 UI-M-AL1-ahj-d-09-0-UI s1 Mus musculus cDNA, 3 end/clone = UI = M-AL1-ahj-d-09-0-UI/ | 118 | P | 308 | P | 162.9 | P |
| | | clone_end = 3/gb = AI844131/gi = 5488037/ug Mm 16767/len = 425/STRA = for | | | | | | |
| 5 | 93267_at | Cluster Incl AA688834: vs04a06 r1 Mus musculus cDNA, 5 end/clone = IMAGE-1137202/clone_end = 5/gb = AA688834/ | 514.5 | P | 672.9 | P | 569.8 | P |
| | | gi = 2678263/ug = Mm 17307/len = 514/STRA = for | | | | | | |
| 5 | 93274_at | Cluster Incl M38381 CDC-like kinase/cds = (85,1536)/gb = M38381/gi = 201070/ug = Mm 1761/len = 1740/STRA = for | 104.2 | P | 406.7 | P | 99 | P |
| 5 | 93278_at | Cluster Incl M91458.Sterol carrier protein 2, liver/cds = (15,1658)/gb = M91458/gi = 293793/ug-Mm 1779/len = 2152/ | 371.8 | P | 552.5 | P | 385.7 | P |
| | | STRA = for | | | | | | |
| 5 | 93337_at | Cluster Incl U10119: Mus musculus SKD1 protein mRNA, complete cds/cds = (229,1563)/gb = U10119/gi = 557875/ | 508.1 | P | 847.3 | P | 551.2 | P |
| | | ug = Mm 18705/len = 2844/STRA = for | | | | | | |
| 5 | 93488_at | Cluster Incl AI840579 UI-M-AM0-adp-h-12-0-UI.s1 Mus musculus cDNA, 3 end/clone = UI-M-AM0-adp-h-12-0-UI/ | 118.6 | P | 243.5 | P | 202.1 | P |
| | | clone_end = 3/gb = AI840579/gi = 5474792/ug = Mm.19015/len = 272/STRA = for | | | | | | |
| 5 | 93491_f_at | Cluster Incl AI836623 UI-M-AP0-abh-a-10-0-UI s1 Mus musculus cDNA, 3 end/clone = UI-M-AP0-abh-a-10-0-UI/ | 469.2 | P | 643.2 | P | 417.5 | P |
| | | clone_end = 3/gb = AI836623/gi = 5470836/ug = Mm 19060/len = 523/STRA = for | | | | | | |
| 5 | 93493_at | Cluster Incl X65627 D-E-A-D (aspartate-glutamate-alanine-aspartate) box polypeptide 5/cds = (160,2004)/gb = X65627/ | 726.3 | P | 1063.3 | P | 781.1 | P |
| | | gi = 51262/ug = Mm.19101/len = 2318/STRA = for | | | | | | |
| 5 | 93501_f_at | Cluster Incl AF058955 Mus musculus ATP-specific succinyl-CoA synthetase beta subunit (Sca) mRNA, partial cds/ | 407.5 | P | 684.6 | P | 468.5 | P |
| | | cds = (0,1280)/gb = AF058955/gi = 3766200/ug = Mm.19154/len = 1991/STRA = for | | | | | | |
| 5 | 93503_at | Cluster Incl U88567: Mus musculus secreted frizzled related protein saFRP-2 (Sfrp2) mRNA, complete cds/cds = (235,1122)/ | 602.1 | P | 390.7 | P | 417.8 | P |
| | | gb = U88567/gi = 1946342/ug = Mm 19155/len = 2028 STRA = for | | | | | | |
| 5 | 93546_s_at | Cluster Incl D14572 Mouse mRNA for PEBP2b1 protein, complete cds/cds = (232,795)/gb = D14572/gi = 303693/ | 1027.5 | P | 1267.6 | P | 1036.5 | P |
| | | ug = Mm 2018/len = 2805/STRA = for | | | | | | |
| 5 | 93572_at | Cluster Incl AI835051 UI-M-AI1-afn-e-02-0-UI s1 Mus musculus cDNA, 3 end/clone-UI-M-AI1-afn-o-02-0-UI/clone_end = 3/ | 488.4 | P | 640.5 | P | 550.7 | P |
| | | gb = AI835051/gi = 5469264/ug = Mm.2041/len = 180/STRA = for | | | | | | |
| 5 | 93773_f_at | Cluster Incl AI835041 UI-M-AI1-afn-d-02-0-UI s1 Mus musculus cDNA, 3 end/clone = UI-M-AI1-afn-d-02-0-UI/clone_end = 3/ | 304.6 | P | 369.3 | P | 234.4 | P |
| | | gb = AF835041/gi = 5469254/ug = Mm 21203/len = 409/STRA = for | | | | | | |
| 5 | 93784_at | Cluster Incl AB010828 Mus musculus mRNA for BCNT, complete cds/cds = (103,990)/gb = AB010828/gi = 3327263/ | 284.8 | P | 347.7 | P | 270.2 | P |
| | | ug = Mm.21310/len = 1215/STRA = for | | | | | | |
| 5 | 93794_at | Cluster Incl AI846393 UI-M-AN1-afa-h-03-0-UI s1 Mus musculus cDNA, 3 end/clone = UI-M-AN1-afa-h-03-0-UI/ | 208.6 | P | 373.7 | P | 323.5 | P |
| | | clone_end = 3/gb = AI846393/gi = 5490299/ug = Mm 21396/len = 193/STRA = for | | | | | | |
| 5 | 93806_at | Cluster Incl AI848671 UI-M-AH1-agv-e-02-0-UI s1 Mus musculus cDNA, 5 end/clone = UI-M-AH1-agv-e-20-0-UI/ | 191.5 | P | 347 | P | 247.7 | P |
| | | clone_end = 3/gb = AI848671/gi = 5492577/ug = Mm.21446/len = 385/STRA = for | | | | | | |
| 5 | 93986_at | Cluster Incl AA939576: vz51e01 r1 Mus musculus cDNA, 5 end/clone = IMAGE-1330008/clone_end = 5/gb = AA939576/ | 464.1 | P | 458.1 | P | 489.2 | P |
| | | gi = 3100353/ug = Mm 21714/len = 400/STRA = for | | | | | | |
| 5 | 93990_at | Cluster Incl Y14196 Mus musculus mRNA for heterogeneous nuclear ribonucleoprotein H/cds = (82,1431)/gb = Y14196/ | 435 | P | 1004.9 | P | 418.2 | P |
| | | gb = 2253040/ug = Mm.21740/len = 2181/STRA = for | | | | | | |
| 5 | 94081_at | Cluster Incl AW124390 UI-M-BH2 1-ape-c-03-0-UI s1 Mus musculus cDNA, 3 end/clone = UI-M-BH2 1-ape-c-03-0-UI/ | 207.2 | P | 271.7 | P | 233.5 | P |
| | | clone_end = 3/gb = AW124390/gi = 6099920/ug = Mm 22162/len = 217/STRA = for | | | | | | |
| 5 | 94250_at | Cluster Incl U14172 Mus musculus p162 protein mRNA, complete cds/cds = (126,4160)/gb = U14172/gi = 1205975/ | 173.7 | P | 552.8 | P | 292 | P |
| | | ug = Mm.2238/len = 5119/STRA = for | | | | | | |
| 5 | 94319_at | Cluster Incl L04966 Mus musculus ras-related protein (rab18) mRNA, complete cds/cds = (4,624)/gb = L04966/gi = 388324/ | 892.8 | P | 1176.2 | P | 948.5 | P |
| | | ug = Mm 22660/len = 692/STRA = for | | | | | | |
| 5 | 94810_at | Cluster Incl X79233: Ewing sarcoma homolog/cds = (56,2023)/gb = X79233/gi = 488512/ug = Mm.22691/len = 2188/STRA = for | 857.3 | P | 871.6 | P | 766.3 | P |
| 5 | 94830_at | Cluster Incl AI854300 UI-M-BH0-ajs-f-06-0-UI.s1 Mus musculus cDNA, 3 end/clone = UI-M-BHO-ajs-f-06-0-UI/clone_end = 3/ | 518.1 | P | 515.4 | P | 486.3 | P |
| | | gb = AI854300/gi = 5498206/ug = Mm.22749/len = 554/STRA = for | | | | | | |
| 5 | 95418_at | Cluster Incl AI848851 UI-M-AJ1-ahb-d-09-0 UI s1 Mus musculus cDNA, 3 end/clone = UI-M-AJ1-ahb-d-09-0-UI/ | 157.1 | P | 317.7 | P | 283.4 | P |
| | | clone_end = 3/gb = AI848851/gi = 5492757/ug = Mm 24348/len = 377/STRA = for | | | | | | |
| 5 | 95432_f_at | Cluster Incl AI844034 UI-M-AL1-ahi-d-02-0-UI s1 Mus musculus cDNA, 3 end/clone = UI-M-AL1-ahi-d-02-0-UI/ | 392.3 | P | 491.8 | P | 427.4 | P |
| | | clone_end = 3/gb = AI844034/gi = 5487940/ug = Mm 24463/len = 404/STRA = for | | | | | | |
| 5 | 95456_r_at | Cluster Incl U41626 Split hand/foot deleted gene 1/cds = (46,258)/gb = U41626/gi = 1209725/ug = Mm 2469/len = 432/ | 1591.2 | P | 1536.7 | P | 1334.8 | P |
| | | STRA = for | | | | | | |
| 5 | 95472_f_at | Cluster Incl AI526902 uj47g07.x1 Mus musculus cDNA, 3 end/clone = IMAGE-1923132/clone_end = 3/gb = AI526902/ | 861.9 | P | 1246.3 | P | 919.2 | P |
| | | gi = 4441037/ug = Mm 24805/len = 484/STRA = for | | | | | | |
| 5 | 95485_at | Cluster Incl D29639 Mouse embryonal carcinoma cell mRNA for 3-hydroxyacyl CoA dehydrogenase, complete cds/ | 946.8 | P | 748.8 | P | 1003.5 | P |
| | | cds = (53,964)/gb = D29639/gi = 1125025/ug = Mm 2491/len = 1679/STRA = for | | | | | | |
| 5 | 95488_at | Cluster Incl AW045756 UI-M-BH1-akt-b-04-0-UI s1 Mus musculus cDNA, 3 end/clone = UI-M-BH1-akt-b-04-0-UI/ | 649.8 | P | 906.8 | P | 688.5 | P |
| | | clone_end = 3/gb = AW045756/gi = 5906285/ug = Mm.25018/len = 357/STRA = for | | | | | | |
| 5 | 95516_at | Cluster Incl AB027290 Mus musculus SID 99 mRNA for small GTP binding protein, complete cds/cds = (215,820)/ | 641.4 | P | 747.6 | P | 716 | P |
| | | gb = AB027290/gi = 5931815/ug = Mm 25306/len = 1276/STRA = for | | | | | | |
| 5 | 96029_at | Cluster Incl AI020542 ub24g02.r1 Mus musculus cDNA, 5 end/clone = IMAGE-1378706/clone_end = 5/gb = AI020542/ | 272.1 | P | 368.4 | P | 329.8 | P |
| | | gi = 3234878/ug = Mm 25779/len = 374/STRA = for | | | | | | |
| 5 | 96096_f_at | Cluster Incl AI648018 uk39c03.x1 Mus musculus cDNA, 3 end/clone = IMAGE-1971364/clone_end = 3/gb = AI648018/ | 166.8 | P | 251.4 | P | 176.8 | P |
| | | gi = 4726696/ug = Mm 26772/len = 543/STRA = for | | | | | | |
| 5 | 96112_at | Cluster Incl AI651178 UI-M-BH0-akg-g-09-UI s1 Mus musculus cDNA, 3 end/clone = UI-M-BH0-akg-g-09-0-UI/ | 764.9 | P | 794.3 | P | 762.3 | P |
| | | clone_end = 3/gb = AI851178/gi = 5495049/ug = Mm.26949/len = 515/STRA = for | | | | | | |
| 5 | 96809_at | Cluster Incl AW122195 UI-M-BH2.2-aov-b-06-0-UI s1 Mus musculus cDNA, 3 end/clone = UI-M-BH2 2-aov-b-06-0-UI/ | 241.8 | P | 504.6 | P | 405.6 | P |
| | | clone_end = 3/gb = AW122195/gi = 6097650/ug = Mm 27074/len = 589/STRA = for | | | | | | |
| 5 | 96651_at | Cluster Incl AF035263 Mus musculus BAF57 (Baf57) gene, complete cds/cds = (0,1235)/gb = AF035283/gi = 2614754/ | 181.2 | P | 197.5 | P | 163.8 | P |
| | | ug = Mm 27330/len = 1236/STRA = for | | | | | | |
| 5 | 96665_at | Cluster Incl AW211618 uo84d01 y1 Mus musculus cDNA, 5 end/clone = IMAGE-2649217/clone_end = 5/gb = AW211618/ | 331.5 | P | 419.9 | P | 366.1 | P |
| | | gi = 6517659/ug = Mm 27373/len = 442/STRA = for | | | | | | |
| 5 | 96688_at | Cluster Incl AI845463 UI-M-AO1-aej-g-06-0-UI.s1 Mus musculus cDNA, 3 end/clone = UI-M-AO1-aej-g-06-0-UI/ | 88.5 | P | 46.8 | P | 94.4 | P |
| | | clone_end = 3/gb = AI845463/gi = 5489369/ug = Mm 27487/len = 423/STRA = for | | | | | | |
| 5 | 97255_at | Cluster Incl AW061318 UI-M-BH1-anw-e-02-0-UI s1 Mus musculus cDNA, 3 end/clone = UI-M-BH1-anw-e-02-0-UI/ | 253.5 | P | 314.9 | P | 212.1 | P |
| | | clone_end = 3/gb = AW061318/gi = 6009069/ug = Mm 27860/len = 402/STRA = for | | | | | | |
| 5 | 97279_at | Cluster Incl AI837615 UI-M-AK0-adg-d-01-0-UI s1 Mus musculus cDNA, 3 end/clone = UI-M-AK0-adg-d-01-0-UI/ | 323.6 | P | 335.1 | P | 319 | P |
| | | clone_end = 3/gb = AI837615/gi = 5471828/ug = Mm 27959/len = 467/STRA = for | | | | | | |
| 5 | 97302_at | Cluster Incl AI854285.UI-M-BH0-ajs-e-02-0-UI s1 Mus musculus cDNA, 3 end/clone = UI-M-BH0-ajs-e-02-0-UI/ | 273.8 | P | 334.9 | P | 238.2 | P |
| | | clone_end = 3/gb = AI854285/gi = 5498191/ug = Mm 28049/len = 427/STRA = for | | | | | | |
| 5 | 97304_at | Cluster Incl AI836100: UI-M-AJ0-abb-h-02-0-UI s1 Mus musculus cDNA, 3 end/clone = UI-M-AJ0-abb-h-02-0-UI/ | 390.5 | P | 401.2 | P | 348.5 | P |
| | | clone_end = 3/gb = AI836100/gi = 5470313/ug = Mm 28052/len = 358/STRA = for | | | | | | |
| 5 | 97812_at | Cluster Incl AF006465 Mus musculus B cell antigen receptor Ig beta associated protein 1 (IBAP-1) mRNA, complete cds/ | 156 | P | 246.6 | P | 159.7 | P |
| | | cds = (45,2006)/gb = AF006465/gi = 4101717/ug = Mm 28165/len = 2190/STRA = for | | | | | | |
| 5 | 97838_at | Cluster Incl AA684508 vr02b07.r1 Mus musculus cDNA, 5 end/clone = IMAGE-1110613/clone_end = 5/gb = AA684508/ | 669.8 | P | 430.7 | P | 442.5 | P |
| | | gi = 2670225/ug = Mm 28239/len = 398/STRA = for | | | | | | |
| 5 | 97839_at | Cluster Incl AI841960.UI-M-AH0-acu-c-10-0-UI.s2 Mus musculus cDNA, 3 end/clone = UI-M-AH0-acu-c-10-0-UI/ | 1317.2 | P | 1343.1 | P | 1109.5 | P |
| | | clone_end = 3/gb = AI841960/gi = 5476173/ug = Mm 28240/len = 335/STRA = for | | | | | | |
| 5 | 97880_at | Cluster Incl AW061024: UI-M-BH1-amn-g-03-0-UI s1 Mus musculus cDNA, 3 end/clone = UI-M-BH1-amn-g-03-0-UI/ | 570.9 | P | 529.8 | P | 460.9 | P |
| | | clone_end = 3/gb = AW061024/gi = 6008775/ug = Mm 28365/len = 457/STRA = for | | | | | | |
| 5 | 97896_i_at | Cluster Incl AW125218: UI-M-BH2.1-aps-c-06-0-UI s1 Mus musculus cDNA, 3 end/clone = UI-M-BH2 1-aps-c-06-0-UI/ | 211.6 | P | 181.9 | P | 195.2 | P |
| | | clone_end = 3/gb = AW125218/gi = 6100748/ug = Mm 28421/len = 319/STRA = for | | | | | | |
| 5 | 97897_at | Cluster Incl AI848435 UI-M-AH1-ags-e-03-0-UI s1 Mus musculus cDNA, 3 end/clone = UI-M-AH1-ags-e-03-0-UI/ | 348.6 | P | 544.3 | P | 459.9 | P |
| | | clone_end = 3/gb = AI848435/gi = 5492341/ug = Mm 28422/len = 500/STRA = for | | | | | | |
| 5 | 98905_at | Cluster Incl AJ223782 Mus musculus CDC10 gene, promoter, exon 1 and joined CDS/cds = (68,1378)/gb = AJ223782/ | 359.6 | P | 907.5 | P | 601.8 | P |
| | | gi = 2827748/ug = Mm 2858/len = 2392/STRA = for | | | | | | |
| 5 | 98909_at | Cluster Incl AW060843: UI-M-BH1-amx-d-06-0-UI s1 Mus musculus cDNA, 3 end/clone = UI-M-BH1-amx-d-06-0-UI/ | 401.8 | P | 403.2 | P | 354.1 | P |
| | | clone_end = 3/gb = AW060843/gi = 6008594/ug = Mm 28591/len = 419/STRA = for | | | | | | |
| 5 | 98946_at | Cluster Incl AF033166 Mus musculus WSB-1 mRNA, complete cds/cds = (0,1265)/gb = AF033186/gi = 2766488/ | 287.3 | P | 171.7 | P | 202.5 | P |
| | | ug = Mm 28719/len = 1266/STRA = for | | | | | | |
| 5 | 94460_at | Cluster Incl AA691445 vs11h05.r1 Mus musculus cDNA, 5 end/clone = IMAGE-1137945/clone_end = 5/gb = AA691445/ | 70.2 | P | 266.3 | P | 225.4 | P |
| | | gi = 2692383/ug = Mm 28821/len = 500/STRA = for | | | | | | |
| 5 | 94505_at | Cluster Incl AW124934 UI-M-BH2 1-apu-g-11-0-UI.s1 Mus musculus cDNA, 3 end/clone = UI-M-BH2.1-apu-g-11-0-UI/ | 394.8 | P | 667 | P | 453.6 | P |
| | | clone_end = 3/gb = AW124934/gi = 6100464/ug = Mm 28957/len = 425/STRA = for | | | | | | |
| 5 | 94507_at | Cluster Incl U15977 Mus musculus long chain fatty acyl CoA synthetase mRNA, complete cds/cds = (0,2099)/gb = U15977/ | 532.3 | P | 723 | P | 701.7 | P |
| | | gi = 563830/ug = Mm 28962/len = 2100/STRA = for | | | | | | |
| 5 | 95077_at | Cluster Incl U12922 RAB geramylgeranyl transferase, b subunit/cds = (0,1019)/gb = U12922/gi = 1345081/ug = Mm 29211/ | 472.7 | P | 502.2 | P | 404.8 | P |
| | | len = 1174/STRA = for | | | | | | |
| 5 | 95096_at | Cluster Incl AI846695 UI-M-AN1-afi-f-06-0-UI s1 Mus musculus cDNA, 3 end/clone = UI-M-AN1-afi-f-06-0-UI/clone_end = 3/ | 983.6 | P | 1378.3 | P | 956.5 | P |
| | | gb = AI846695/gi = 5490601/ug = Mm.29313/len = 374/STRA = for | | | | | | |
| 5 | 95101_at | Cluster Incl AI834772: UI-M-AM0-ads-b-10-0-UI s1 Mus musculus cDNA, 3 end/clone = UI-M-AM0-ads-b-10-0-UI/ | 309.5 | P | 545.3 | P | 307.2 | P |
| | | clone_end = 3/gb = AI834772/gi = 5469005/ug = Mm 29344/len = 401/STRA = for | | | | | | |
| 5 | 95647_f_at | Cluster Incl AI485345: vw50c09 y1 Mus musculus cDNA, 5 end/clone = IMAGE-1247248/clone_end = 5/gb = AI465845 | 478.6 | P | 722.2 | P | 596.6 | P |
| | | gi = 4319875/ug = Mm 29500/len = 327/STRA = for | | | | | | |
| 5 | 95648_at | Cluster Incl AA655507: vs25d01.r1 Mus musculus cDNA, 5 end/clone = IMAGE-1139233/clone_end = 5/gb = AA655507/ | 549.5 | P | 689.3 | P | 568.6 | P |
| | | gi = 2591661/ug = Mm.29500/len = 522/STRA = for | | | | | | |
| 5 | 95675_at | Cluster Incl AW121182 UI-M-BH2 3-aok-a-10-0-UI s1 Mus musculus cDNA, 3 end/cone = UI-M-BH2 3-aok-a-10-0-UI/ | 219.5 | P | 341.8 | P | 255.5 | P |
| | | clone_end = 3/gb = AW121182/gi = 6096515/ug = Mm 29588/len = 516/STRA = for | | | | | | |
| 5 | 96256_at | Cluster Incl M28723 Anti-oxidant protein 1/cds = (47,820)/gb = M28723/gi = 309435/ug = Mm 29821/len = 1382/STRA = for | 243.1 | P | 406 | P | 417.5 | P |
| 5 | 96268_at | Cluster Incl AI840979 UI-M-AK0-adk-e-11-0-UI s1 Mus musculus cDNA,3 end/clone = UI-M-AK0-adk-e-11-0-UI/ | 675.2 | P | 820.5 | P | 892 | P |
| | | clone_end = 3/gb = AI840979/gi = 5475192/ug = Mm.29845/len = 405/STRA = for | | | | | | |
| 5 | 96862_at | Cluster Incl AI848584 UI-M-AM1-aga-d-04-0-UI s2 Mus musculus cDNA, 3 end/clone = UI-M-AM1-aga-d-04-0-UI/ | 1031.5 | P | 1442.4 | P | 1278 | P |
| | | clone_end = 3/gb = AI848584/gi = 5492490/ug = Mm 30053/len = 305/STRA = for | | | | | | |
| 5 | 96871_at | Cluster Incl AI844355 UI-M-AL1-ahp-a-08-0-UI s1 Mus musculus cDNA, 3 end/clone = UI-M-AL1-ahp-a-08-0-UI/ | 380.3 | P | 463.8 | P | 394.3 | P |
| | | clone_end = 3/gb = AI844355/gi = 5488261/ug = Mm 30065/len = 330/STRA = for | | | | | | |
| 5 | 96875_r_at | Cluster Incl AI841410: UI-M-AH0-acx-b-06-0-UI s1 Mus musculus cDNA, 3 end/clone = UI-M-AH0-acx-b-06-0-UI/ | 76.2 | P | 179.4 | P | 88.5 | P |
| | | clone_end = 3/gb = AI841410/gi = 5475623/ug = Mm 30069/len = 286/STRA = for | | | | | | |
| 5 | 96881_at | Cluster Incl AW049394 UI-M-BH1-ane-e-07-0-UI s1 Mus musculus cDNA, 3 end/clone = UI-M-BH1-ane-e-07-0-UI/ | 406 | P | 352.7 | P | 418.9 | P |
| | | clone_end = 3/gb = AW049394/gi = 5909923/ug = Mm 30077/len = 543/STRA = for | | | | | | |
| 5 | 96886_at | Cluster Incl AW060556 UI-M-BH1-ann-h-02-0-UI s1 Mus musculus cDNA, 3 end/clone = UI-M-BH1-ann-h-02-0-UI/ | 185.6 | P | 147.4 | P | 158.3 | P |
| | | clone_end = 3/gb = AW060556/gi = 6008307/ug = Mm.30083/len = 319/STRA = rev | | | | | | |
| 5 | 96909_at | Cluster Incl AI849803 UI-M-AL1-aho-b-03-0-UI s1 Mus musculus cDNA, 3 end/clone = UI-M-AL1-aho-b-03-0-UI/ | 731.4 | P | 796.5 | P | 850 | P |
| | | clone_end = 3/gb = AI849803/gi = 5493709/ug = Mm.3014/len = 501/STRA = for | | | | | | |
| 5 | 97448_at | Cluster Incl AI845165 UI-M-BG0-ahs-g-10-0-UI s1 Mus musculus cDNA, 3 end/clone = UI-M-BG0-ahs-g-10-0-UI/ | 457.9 | P | 548 | P | 495.8 | P |
| | | clone_end = 3/gb = AI845165/gi = 5489071/ug = Mm.30245/len = 214/STRA = for | | | | | | |
| 5 | 97491_at | Cluster Incl AW123032 UI-M-BH2 1-apb-g-02-0-UI s1 Mus musculus cDNA, 3 end/clone = UI-M-BH2 1-apb-g-02-0-UI/ | 183.3 | P | 338.9 | P | 146.8 | P |
| | | clone_end = 3/gb = AW123032/gi = 6098562/ug = Mm 31051/len = 361/STRA = for | | | | | | |
| 5 | 97502_at | Cluster Incl U73445: Dihydrolipoamide dehydrogenase/cds = (109,1638)/gb = U73445/gi = 2078521/ug = Mm 3131/len = 2163/ | 939.2 | P | 1495 | P | 1211.9 | P |
| | | STRA = for | | | | | | |
| 5 | 98065_at | Cluster Incl AI837679 UI-M-AK0-adj-b-10-0-UI.s1 Mus musculus cDNA, 3 end/clone = UI-M-AK0-adj-b-10-0-UI/ | 932 | P | 973.9 | P | 751.3 | P |
| | | clone_end = 3/gb = AI837679/gi = 5471892/ug = Mm 34416/len = 159/STRA = for | | | | | | |
| 5 | 98087_at | Cluster Incl AW048562 UI-M-BH1-alx-e-05-0-UI s1 Mus musculus cDNA, 3 end/clone = UI-M-BH1-alx-e-05-0-UI/ | 199.6 | P | 335.3 | P | 262.5 | P |
| | | clone_end = 3/gb = AW048562/gi = 5909091/ug = Mm 34580/len = 474/STRA = for | | | | | | |
| 5 | 98121_at | Cluster Incl D49744 Farnesyltransferase, CAAX box, alpha/cds = (15,1148)/gb = D49744/gi = 1136744/ug = Mm.3496/ | 603.2 | P | 440.9 | P | 594 | P |
| | | len = 1629/STRA = for | | | | | | |
| 5 | 98127_at | Cluster Incl U16741 Capping protein alpha 2/cds = (105,965)/gb = U16741/gi = 595918/ug = Mm.3529/len = 1690/STRA = for | 373.1 | P | 386.8 | P | 430.9 | P |
| 5 | 98524_f_at | Cluster Incl AI848479: UI-M-AP1-agf-a-10-0-UI.s2 Mus musculus cDNA, 3 end/clone = UI-M-AP1-agf-a-10-0-UI/ | 305.7 | P | 404.5 | P | 245.6 | P |
| | | clone_end = 3/gb = AI848479/gi = 5492385/ug = Mm 35823/len = 420/STRA = for | | | | | | |
| 5 | 98531_g_at | Cluster Incl AI849615: UI-M-AH1-agr-g-09-0-UI s1 Mus musculus cDNA, 3 end/clone = UI-M-AH1-agr-g-09-0-UI/ | 691 | P | 1509.9 | P | 881.1 | P |
| | | clone_end = 3/gb = AI849615/gi = 5493521/ug = Mm 35844/len = 337/STRA = for | | | | | | |
| 5 | 98540_g_at | Cluster Incl AF071312 Mus musculus COP9 complex subunit 2 (COPS2) mRNA, complete cds/cds = (12,1334)/ | 116.3 | P | 220.4 | P | 134 | P |
| | | gb = AF071312/gi = 3309165/ug = Mm.3596/len = 1929/STRA = for | | | | | | |
| 5 | 98553_at | Cluster Incl AW124175: UI-M-BH2 1-apr-h-11-0-UI s1 Mus musculus cDNA, 3 end/clone = UI-M-BH2 1-apr-h-11-0-UI/ | 140.7 | P | 311.4 | P | 232.9 | P |
| | | clone_end = 3/gb = AW124175/gi = 6099705/ug = Mm 36769/len = 208/STRA = for | | | | | | |
| 5 | 98587_at | Cluster Incl X61449: M. musculus mRNA for clone DN38/cds = (0,1034)/gb = X61449/gi = 50721/ug = Mm 3797/len = 1322/ | 582.5 | P | 595 | P | 507.7 | P |
| | | STRA = for | | | | | | |
| 5 | 98595_at | Cluster Incl U56773 Interleukin 1 receptor-associated kinase/cds = (15,2048)/gb = U56773/gi = 1480173/ug = Mm.38241/ | 361 | P | 346.6 | P | 290.5 | P |
| | | len = 2703/STRA = for | | | | | | |
| 5 | 99095_at | Cluster Incl M63903: Max protein/cds = (160,642)/gb = M63903/gi = 199986/ug = Mm 3931/len = 1941/STRA = for | 202.4 | P | 293.1 | P | 273.7 | P |
| 5 | 99577_at | Cluster Incl M57647: Mouse mast cell growth factor (MGF) mRNA, complete cds/cds = (149,970)/gb = M57647/gi = 19951/ | 102.5 | P | 375.9 | P | 199.4 | P |
| | | ug = Mm 4235/len = 2062/STRA = for | | | | | | |
| 5 | 99604_at | Cluster Incl AI848713: UI-M-AJ1-ahf-h-10-0-UI.s1 Mus musculus cDNA, 3 end/clone = UI-M-AJ1-ahf-a-10-0-UI/ | 546 | P | 795.4 | P | 767.2 | P |
| | | clone_end = 3/gb = AI848713/gi = 5492619/ug = Mm 42925/len = 437/STRA = for | | | | | | |
| 5 | 99621_s_at | Cluster Incl AA690583: vu53c05.r1 Mus musculus cDNA, 5 end/clone = IMAGE-1195112/clone_end = 5/gb = AA690583/ | 248.2 | P | 297 | P | 271.3 | P |
| | | gi = 2691519/ug = Mm 43213/len = 424/STRA = for | | | | | | |
| 5 | 99628_at | Cluster Incl AF020308 Mus musculus HRS gene, complete cds/cds = (0,812)/gb = AF020308/gi = 2435502/ug = Mm 43331/ | 473.2 | P | 590.7 | P | 222.8 | P |
| | | len = 813/STRA = for | | | | | | |
| 5 | 99649_at | Cluster Incl U85414 Glutamate cysteine ligase (gamma-glutamylcysteine synthetase), catalytic/cds = (0,1913)/gb = U85414/ | 178.3 | P | 165.3 | P | 136.7 | P |
| | | gi = 1815761/ug = Mm 4368/len = 1914/STRA = for | | | | | | |
| 5 | 100089_at | Cluster Incl M74227 Mouse cyclophilin C (cyp C) mRNA, complete cds/cds = (51,689)/gb = M74227/gi = 192898/ | 1084.6 | P | 907.7 | P | 640.3 | P |
| | | ug = Mm 4587/len = 1287/STRA = for | | | | | | |
| 5 | 100534_at | Cluster Incl AI183109: ub91h12 r1 Mus musculus cDNA, 5 end/clone = IMAGE-1395911/clone_end = 5/gb = AI183109/ | 562.4 | P | 617.8 | P | 524.2 | P |
| | | gi = 3733747/ug = Mm 5248/len = 358/STRA = for | | | | | | |
| 5 | 100543_s_at | Cluster Incl AA620184: vo63e07.r1 Mus musculus cDNA, 5 end/clone = IMAGE-1054596/clone_end = 5/gb = AA620184/ | 258.3 | P | 248.4 | P | 201.1 | P |
| | | gi = 2524060/ug = Mm 5400/len = 507/STRA = for | | | | | | |
| 5 | 100584_at | Cluster Incl U72941 Mus musculus annexin IV mRNA, complete cds/cds = (90,1049)/gb = U72941/gi = 1778312/ug = Mm 619/ | 388.4 | P | 353.9 | P | 384 | P |
| | | len = 1961/STRA = for | | | | | | |
| 5 | 100608_at | Cluster Incl X95641: M. musculus mRNA for serine palmitoyltransferase subunit A/cds = (11,1432)/gb = X95641/gi = 2564281/ | 1189.1 | P | 1338.1 | P | 917.5 | P |
| | | ug = Mm 6505/len = 2576/STRA = for | | | | | | |
| 5 | 101484_at | Cluster Incl U73039 Next to the Brca1/cds = (290,3256)/gb = U73039/gi = 1815651/ug = Mm.764/len = 4444/STRA = for | 329.5 | P | 454.8 | P | 318.7 | P |
| 5 | 101584_at | Cluster Incl X636039 M musculus RSP-1 mRNA for p33 protein/cds = (34,867)/gb = X63039/gi = 54014/ug = Mm.905/ | 439.6 | P | 450.7 | P | 328.2 | P |
| | | len = 1327/STRA = for | | | | | | |
| 5 | 101590_at | Cluster Incl AI747194 ul13a12 x1 Mus musculus cDNA, 3 end/clone = IMAGE-2076670/clone_end = 3/gb = AI747194/ | 736.7 | P | 1228.2 | P | 1001.4 | P |
| | | gi = 5125458/ug = Mm 91146/len = 585/STRA = for | | | | | | |
| 5 | 101971_at | Cluster Incl AW061255: UI-M-BH1-amj-e-08-0-UI.s2 Mus musculus cDNA, 3 end/clone = UI-M-BH1-amj-e-08-0-UI/ | 258.8 | P | 367.4 | P | 252.7 | P |
| | | clone_end = 3/gb = AW061255/gi = 6009006/ug = Mm.9495/len = 495/STRA = for | | | | | | |
| 5 | 102381_at | Cluster Incl AA619207: vo73e09.r1 Mus musculus cDNA, 5 end/clone = IMAGE-1064776/clone_end = 5/gb = AA619207/ | 112.9 | P | 216.2 | P | 163.9 | P |
| | | gi = 2523083/ug = Mm 12166/len = 367/STRA = for | | | | | | |
| 5 | 102427_at | Cluster Incl AI849991 UI-M-BG0-aib-d-09-0-UI s1 Mus musculus cDNA, 3 end/clone = UI-M-BG0-aib-d-09-0-UI/ | 387.3 | P | 609.3 | P | 580.8 | P |
| | | clone_end = 3/gb = AI849991/gi = 5493897/ug = Mm.12831/len = 446/STRA = for | | | | | | |
| 5 | 102781_at | Cluster Incl U37351 Mus musculus Paneth cell enhanced expression PCEE mRNA, complete cds/cds = (110,670)/ | 306.7 | P | 492.9 | P | 313.7 | P |
| | | gb = U37351/gi = 1276644/ug = Mm 13725/len = 1220/STRA = for | | | | | | |
| 5 | 102850_at | Cluster Incl AF037260 Mus musculus non-receptor protein tyrosine kinase Ack mRNA, complete cds/cds = (267,3434)/ | 218.4 | P | 521.2 | P | 400.8 | P |
| | | gb = AF037260 gi = 2921446/ug = Mm 1483/len = 4214/STRA = for | | | | | | |
| 5 | 102990_at | Cluster Incl AA655199: vv13b10.r1 Mus musculus cDNA, 5 end/clone = IMAGE-1211515/clone_end = 5/gb = AA655199/ | 1111.7 | P | 866.6 | P | 929.1 | P |
| | | gi = 2591353/ug = Mm.1543/len = 367/STRA = for | | | | | | |
| 5 | 103027_at | Cluster Incl AA739413 vv53b11 r1 Mus musculus cDNA, 5 end/clone = IMAGE-1226109/clone_end = 5/gb = AA739413/ | 228.2 | P | 208.9 | P | 207.8 | P |
| | | gi = 2775599/ug = Mm.15974/len = 388/STRA = for | | | | | | |
| 5 | 103097_at | Cluster Incl AI844487: UI-M-AJ1-ahe-e-12-0-UI.s1 Mus musculus cDNA, 3 end/clone = UI-M-AJ1-ahe-e-12-0-UI/ | 151.2 | P | 141.3 | P | 174.2 | P |
| | | clone_end = 3/gb = AI844487/gi = 5488393/ug = Mm 17523/len = 363/STRA = for | | | | | | |
| 5 | 103404_at | Cluster Incl AW122831: UI-M-BH2 1-aoz-d-08-0-UI s1 Mus musculus cDNA, 3 end/clone = UI-M-BH2 1-aoz-d-08-0-UI/ | −46.9 | P | 297.8 | P | 229.4 | P |
| | | clone_end = 3/gb = AW122831/gi = 6098361/ug = Mm 1878/len = 376/STRA = for | | | | | | |
| 5 | 103574_at | Cluster Incl AI841606: UI-M-AP0-abn-h-07-0-UI s1 Mus musculus cDNA, 3 end/clone = UI-M-AP0-abn-h-07-0-UI/ | 603.1 | P | 602.4 | P | 570.6 | P |
| | | clone_end = 3/gb = AI841606/gi = 5475819/ug = Mm 1947/len = 378/STRA = for | | | | | | |
| 5 | 103667_at | Cluster Incl AA866655 ud06h05 r1 Mus musculus cDNA, 5 end/clone = IMAGE-1434393/clone_end = 5/gb = AA866655/ | 122.4 | P | 355.2 | P | 238.2 | P |
| | | gi = 2962100/ug = Mm 20722/len = 542/STRA = for | | | | | | |
| 5 | 103875_at | Cluster Incl AA967717: uh03f10.r1 Mus musculus cDNA, 5 end/clone = IMAGE-1616875/clone_end = 5/gb = AA967717/ | 164.4 | P | 92.7 | P | 131.6 | P |
| | | gi = 3141610/ug = Mm 21175/len = 433/STRA = for | | | | | | |
| 5 | 103891_i_at | Cluster Incl AI197161: ue51h10.r1 Mus musculus cDNA, 5 end/clone = IMAGE-1494691/clone_end = 5/gb = AI197161/ | 685.3 | P | 934.4 | P | 774.3 | P |
| | | gi = 3749767/ug = Mm 21288/len = 475/STRA = for | | | | | | |
| 5 | 104056_at | Cluster Incl AI573367: ud42d02.y1 Mus musculus cDNA, 5 end/clone = IMAGE-1448547/clone_end = 5/gb = AI573367/ | 127.4 | P | 311.1 | P | 283.3 | P |
| | | gi = 4536741/ug = Mm 21532/len = 448/STRA = for | | | | | | |
| 5 | 104063_at | Cluster Incl AI840130 UI-M-AO0-acc-a-09-0-UI s1 Mus musculus cDNA, 3 end/clone = UI-M-AO0-acc-a-09-0-UI/ | 186.8 | P | 173.6 | P | 166.9 | P |
| | | clone_end = 3/gb = AI840130/gi = 5474343/ug = Mm 21586/len = 421/STRA = for | | | | | | |
| 5 | 104071_at | Cluster Incl AI852433 UI-M-BH0-ajo-g-04-0-UI.s1 Mus musculus cDNA, 3 end/clone = UI-M-BH0-ajo-g-04-0-UI/ | 928.1 | P | 863.5 | P | 970.1 | P |
| | | clone_end = 3/gb = AI852433/gi = 5496304/ug = Mm 21639/len = 544/STRA = for | | | | | | |
| 5 | 104114_at | Cluster Incl AW122450 UI-M-BH2 2-aou-a-06-0-UI.s1 Mus musculus cDNA, 3 end/clone = UI-M-BH2.2-aou-a-06-0-UI/ | 128.9 | P | 286 | P | 225.2 | P |
| | | clone_end = 3/gb = AW122450/gi = 6097969/ug = Mm 21954/len = 288/STRA = for | | | | | | |
| 5 | 104327_at | Cluster Incl AA755234 vr92d02 r1 Mus musculus cDNA, 5 end/clone = IMAGE-136163/clone_end = 5/gb = AA755234/ | 154.5 | P | 312.1 | P | 187 | P |
| | | gi = 2802432/ug = Mm 22459/len = 176/STRA = for | | | | | | |
| 5 | 104332_at | Cluster Incl AI842242: UI-M-AI1-afp-d-07-0-UI s1 Mus musculus cDNA, 3 end/clone = UI-M-AI1-afp-d-07-0-UI/clone_end = 3/ | 488.5 | P | 516.9 | P | 509 | P |
| | | gb = AI842242/gi = 5476455/ug = Mm 22484/len = 349/STRA = for | | | | | | |
| 5 | 104367_at | Cluster Incl AW124983 UI-M-BH2 1-apv-d-05-0-UI.s1 Mus musculus cDNA, 3 end/clone = UI-M-BH2 1-apv-d-05-0-UI/ | 204.2 | P | 322.8 | P | 230.7 | P |
| | | clone_end = 3/gb = AW124983/gi = 6100513/ug = Mm 22627/len = 398/STRA = for | | | | | | |
| 5 | 104602_at | Cluster Incl AW121972 UI-M-BH2 3-aoj-a-09-0-UI s1 Mus musculus cDNA, 3 end/clone = UI-M-BH2 3-aoj-a-09-0-UI/ | 121.5 | P | 14.2 | P | 104.7 | P |
| | | clone_end = 3/gb = A121972/gi = 6097329/ug = Mm.24110/len = 474/STRA = for | | | | | | |
| 5 | 104609_at | Cluster Incl AA644817: vs82f12 r1 Mus musculus cDNA, 5 end/clone = IMAGE-1152815/clone_end = 5/gb = AA644817/ | 459.8 | P | 526 | P | 416.5 | P |
| | | gi = 2571246/ug = Mm 24148/len = 382/STRA = for | | | | | | |
| 5 | 104618_at | Cluster Incl AI845819 UI-M-AO1-aeg-b-07-0-UI.s1 Mus musculus cDNA, 3 end/clone = UI-M-AO1-aeg-b-07-0-UI/ | 158.5 | P | 145.5 | P | 148.4 | P |
| | | clone_end = 3/gb = AI845819/gi-5489725/ug = Mm 24216/len = 353/STRA = for | | | | | | |
| 5 | 104621_at | Cluster Incl AW046060 UI-M-BH1-akv-d-12-0-UI s1 Mus musculus cDNA, 3 end/clone = UI-M-BH1-akv-d-12-0-UI/ | 739.9 | P | 764.8 | P | 745.6 | P |
| | | clone_end = 3/gb = AW046060/gi = 5906589/ug = Mm 24236/len = 322/STRA = for | | | | | | |
| 5 | 104717_at | Cluster Incl AI848330 UI-M-AH1-ago-b-10-0-UI s1 Mus musculus cDNA, 3 end/clone = UI-M-AH1-ago-b-10-0-UI/ | 152.8 | P | 275.8 | P | 242.8 | P |
| | | clone_end = 3/gb = AI848330/gi = 5492236/ug = Mm 24505/len = 456/STRA = for | | | | | | |
| 5 | 94399_at | Cluster Incl AI843172 UI-M-AK1-aet-b-11-0-UI s1 Mus musculus cDNA, 3 end/clone = UI-M-AK1-aet-b-11-0-UI/ | 242.2 | P | 232.7 | P | 96.3 | P |
| | | clone_end = 3/gb = AI843172/gi = 5477385/ug = Mm 25777/len = 383/STRA = for | | | | | | |
| 5 | 94412_at | Cluster Incl AJ223733 Mus musculus CDK2 gene intron V containing the alternatively spliced exon Ve/cds = (0,123)/ | 471.1 | P | 554.8 | P | 538.5 | P |
| | | gb = AJ223733/gi = 2842420/ug = Mm 260/len = 124/STRA = for | | | | | | |
| 5 | 94413_at | Cluster Incl AW21487: UI-M-BH2 2-aon-h-09-0-UI.s1 Mus musculus cDNA, 3 end/clone = UI-M-BH2.2-aon-h-09-0-UI/ | 213.6 | P | 194 | P | 269.2 | P |
| | | clone_end = 3/gb = AW121487/gi = 6096820/ug = Mm 26089/len = 168/STRA = rev | | | | | | |
| 5 | 94435_at | Cluster Incl AI839117: UI-M-AO0-ach-b-04-0-UI.s1 Mus musculus cDNA, 3 end/clone = UI-M-AO0-ach-b-04-0-UI/ | 60.6 | P | 151 | P | 148.2 | P |
| | | clone_end = 3/gb = AI839117/gi = 5473330/ug = Mm 26537/len = 441/STRA = for | | | | | | |
| 5 | 94439_at | Cluster Incl AW124363 UI-M-BH2 1-apq-h-10-0-UI s1 Mus musculus cDNA, 3 end/clone = UI-M-BH2 1-apq-h-10-0-UI/ | 219.4 | P | 301.4 | P | 257.7 | P |
| | | clone_end = 3/gb = AW124363/gi = 6099893/ug = Mm 26564/len = 253/STRA = for | | | | | | |
| 5 | 94975_at | Cluster Incl AI845607: UI-M-AQ1-adx-e-10-0-UI s1 Mus musculus cDNA, 3 end/clone = UI-M-AQ1-adx-e-10-0-UI/ | 103.4 | P | 158 | P | 136 | P |
| | | clone_end = 3/gb = AI845607/gi = 5489513/ug = Mm 27243/len = 370/STRA = for | | | | | | |
| 5 | 95021_at | Cluster Incl AW124028: UI-M-BH2.1-apn-c-06-0-UI s1 Mus musculus cDNA, 3 end/clone = UI-M-BH2 1-apn-c-06-0-UI/ | 383.8 | P | 423.8 | P | 418.2 | P |
| | | clone_end = 3/gb = AW124028/gi = 6099558/ug = Mm 27476/len = 440/STRA = for | | | | | | |
| 5 | 96130_at | Cluster Incl AI822531: vw13g10 r1 Mus musculus cDNA, 5 end/clone = IMAGE-1243746/clone_end = 5/gb = AA822531/ | 513.9 | P | 730.3 | P | 509.8 | P |
| | | gi = 2892399/ug = Mm 28133/len = 426/STRA = for | | | | | | |
| 5 | 96191_at | Cluster Incl AI852124: UI-M-BH0-ajb-c-05-0-UI s1 Mus musculus cDNA, 3 end/clone = UI-M-BH0-ajb-c-05-0-UI/ | 321.7 | P | 529.5 | P | 509.7 | P |
| | | clone_end = 3/gb = AI852124/gi = 546030/ug = Mm 28512/len = 275/STRA = for | | | | | | |
| 5 | 96192_at | Cluster Incl AF062567: Mus musculus transcription factor Sp3 mRNA, partial cds/cds = (0,2177)/gb = AF062567/gi = 3132609/ | 368.5 | P | 584.3 | P | 491.4 | P |
| | | ug = Mm 28513/len = 3616/STRA = for | | | | | | |
| 5 | 96199_at | Cluster Incl AF073996 Mus musculus myotubularin (Mtm1) mRNA, complete cds/cds = (75,1886)/gb = AF073996/ | 89.8 | P | 105.3 | P | 90.8 | P |
| | | gi = 3882999/ug = Mm.28580/len = 3339/STRA = for | | | | | | |
| 5 | 96238_at | Cluster Incl AI853996: UI-M-BH0-aiv-g-11-0-UI.s1 Mus musculus cDNA, 3 end/clone = UI-M-BH0-aiv-g-11-0-UI/ | 122.8 | P | 173.7 | P | 131.5 | P |
| | | clone_end = 3/gb = AI853996/gi = 5497902/ug = Mm.28807/len = 255/STRA = for | | | | | | |
| 5 | 96726_at | Cluster Incl AW049619: UI-M-BH1-and-d-10-0-UI.s1 Mus musculus cDNA, 3 end/clone = UI-M-BH1-and-d-10-0-UI/ | 191.5 | P | 309.5 | P | 263.1 | P |
| | | clone_end = 3/gb = AW049619/gi = 5910148/ug = Mm.28837/len = 423/STRA = for | | | | | | |
| 5 | 96751_at | Cluster Incl AI842674 UI-M-AO1-aeo-a-12-0-UI.s1 Mus musculus cDNA, 3 end/clone = UI-M-AO1-aeo-a-12-0-UI/ | 969 | P | 1345.2 | P | 1309.8 | P |
| | | clone_end = 3/gb = AI842674/gi = 5476887/ug = Mm.28972/len = 341/STRA = for | | | | | | |
| 5 | 96762_at | Cluster Incl AW046160 UI-M-BH1-akw-b-02-0-UI s1 Mus musculus cDNA, 3 end/clone = UI-M-BH1-akw-b-02-0-UI/ | 617.8 | P | 928 | P | 718.7 | P |
| | | clone_end = 3/gb = AW046160/gi = 5906689/ug = Mm 29005/len = 532/STRA = rev | | | | | | |
| 5 | 96777_at | Cluster Incl AW049372: UI-M-BH1-ane-b-10-0-UI.s1 Mus musculus cDNA, 3 end/clone = UI-M-BH1-ane-b-10-0-UI/ | 175.6 | P | 315.3 | P | 232.3 | P |
| | | clone_end = 3/gb = AW049372/gi = 5909901/ug = Mm 29056/len = 322/STRA = for | | | | | | |
| 5 | 97948_at | Cluster Incl M26391 Retinoblastoma 1/cds = (98,2863)/gb = M26391/gi = 200452/ug = Mm.304/len = 4591/STRA = for | 74.8 | P | 120.8 | P | 80.5 | P |
| 5 | 98455_at | Cluster Incl U46026 Activating transcription factor 2/cds = (0,731)/gb = U46026/gi = 1655802/ug = Mm 3466/len = 969/ | 45.1 | P | 162 | P | 66.2 | P |
| | | STRA = for | | | | | | |
| 5 | 98503_at | Cluster Incl U53586 Ecotropic viral integration site 5/cds = (68,2497)/gb = U53586/gi = 1777918/ug = Mm 35796/len = 5870/ | 133.3 | P | 320.1 | P | 208.1 | P |
| | | STRA = for | | | | | | |
| 5 | 98981_s_at | Cluster Incl X64840: Transcription factor 12/cds = (66,2186)/gb = X64840/gi = 49924/ug = Mm 36894/len = 3007/STRA = for | 623.9 | P | 808.5 | P | 666.2 | P |
| 5 | 99013_f_at | Cluster Incl AI846797: UI-M-AO1-ael-h-03-0-UI.s1 Mus musculus cDNA, 3 end/clone = UI-M-AO1-ael-h-03-0-UI/ | 1833.6 | P | 2314.9 | P | 2163.2 | P |
| | | clone_end = 3/gb = AI846797/gi = 5490703/ug = Mm 38445/len = 383/STRA = for | | | | | | |
| 5 | 99070_at | Cluster Incl U12473 Conserved helix-loop-helix ubiquitous kinase/cds = (53,2290)/gb = U12473/gi = 1079492/ug = Mm 3996/ | 149.8 | P | 363.1 | P | 199.7 | P |
| | | len = 3466/STRA = for | | | | | | |
| 5 | 99491_at | Cluster Incl U53696 interleukin 10 receptor, beta/cds = (2,1051)/gb = U53696/gi = 1305488/ug = Mm.4154/len = 1670/ | 555.1 | P | 637.2 | P | 584.5 | P |
| | | STRA = for | | | | | | |
| 5 | 99493_at | Cluster Incl AI842066 UI-M-AN1-afg-a-11-0-UI s1 Mus musculus cDNA, 3 end/clone = UI-M-AN1-afg-a-11-0-UI/ | 114.9 | P | 166.3 | P | 117.2 | P |
| | | clone_end = 3/gb = AI842066/gi = 5476279/ug = Mm 41555/len = 302/STRA = for | | | | | | |
| 5 | 99513_at | Cluster Incl M72394 Mouse calcium-dependent phospholipid binding protein (PLA2) mRNA, complete cds/cds = (108,2354)/ | 147.1 | P | 185.5 | P | 177.8 | P |
| | | gb = M72394/gi = 200390/ug = Mm.4186/len = 2787/STRA = for | | | | | | |
| 5 | 99552_at | Cluster Incl U79550.Slug, chicken homolog/cds = (43,852)/gb = U79550/gi = 1711202/ug = Mm 4272/len = 1945/STRA = for | 907.4 | P | 1092.3 | P | 678.7 | P |
| 5 | 99955_at | Cluster Incl U70674: Mus musculus m-Numb (m-nb) mRNA, complete cds/cds = (166,1947)/gb = U70674/gi = 1575755/ | 96 | P | 131.4 | P | 121.8 | P |
| | | ug = Mm 4390/len = 3331/STRA = for | | | | | | |
| 5 | 99964_at | Cluster Incl AW061016 UI-M-BH1-amn-f-06-0-UI s1 Mus musculus cDNA, 3 end/clone = UI-M-BH1-amn-f-06-0-UI/ | 39.9 | P | 80.7 | P | 86.7 | P |
| | | clone_end = 3/gb = AW061016/gi = 6008767/ug = Mm 44170/len = 435/STRA = for | | | | | | |
| 5 | 99994_at | Cluster Incl AF041376: Mus musculus cell death activator CIDE-A (Cide-a) mRNA, complete cds/cds = (213,868)/ | 132.5 | P | 475.6 | P | 560.6 | P |
| | | gb = AF041376/gi = 3114591/ug = Mm 449/len = 1114/STRA = for | | | | | | |
| 5 | 100019_at | Cluster Incl D45889 Chondroitin sulfate proteoglycan 2/cds = (0,124)/gb = D45889/gi = 994777/ug = Mm.4575/len = 2201/ | 310.2 | P | 310.9 | P | 258.3 | P |
| | | STRA = for | | | | | | |
| 5 | 100878_at | Cluster Incl AW124985: UI-M-BH2.1-apv-d-07-0-UI.s1 Mus musculus cDNA, 3 end/clone = UI-M-BH2-1-apv-d-07-0-UI/ | 223.7 | P | 343.2 | P | 386.6 | P |
| | | clone_end = 3/gb = AW124985/gi = 6100515/ug = Mm 531/len = 339/STRA = for | | | | | | |
| 5 | 100905_at | Cluster Incl AI849361 UI-M-AJ1-aha-g-09-0-UI s1 Mus musculus cDNA, 3 end/clone = UI-M-AJ1-aha-g-09-0-UI/ | 187.8 | P | 139.5 | P | 218.4 | P |
| | | clone_end = 3/gb = AI849361/gi = 5493267/ug = Mm 5772/len = 132/STRA = for | | | | | | |
| 5 | 100951_at | Cluster Incl AF014010.Polycystic kidney disease 2/cds = (83,2983)/gb = AF014010/gi = 2558834/ug = Mm 6442/len = 5134/ | 105.1 | P | 153.9 | P | 109.7 | P |
| | | STRA = rev | | | | | | |
| 5 | 100957_at | Cluster Incl AA881160 vz07b02 r1 Mus musculus cDNA, 5 end/clone = IMAGE-1314987/clone_end = 5/gb = AA881160/ | 212.9 | P | 232.3 | P | 186.3 | P |
| | | gi = 2990470/ug = Mm 6509/len = 575/STRA = for | | | | | | |
| 5 | 100984_at | Cluster Incl M63725 Mouse binding protein for T-cell receptor (TCR-ATF1) mRNA, complete cds/cds = (20,829)/ | 234.4 | P | 295.8 | P | 293.2 | P |
| | | gb = M63725/gi = 201821/ug = Mm.676/len = 2122/STRA = for | | | | | | |
| 5 | 101906_at | Cluster Incl AA032310: MTA.G11.085 A Mus musculus cDNA, 5 end/clone = MTA.G11.085/clone_end = 5/gb = AA032310/ | 337.4 | P | 486.2 | P | 315.1 | P |
| | | gi = 1504156/ug = Mm.89817/len = 380/STRA = for | | | | | | |
| 5 | 101914_at | Cluster Incl AI846484 UI-M-AN1-aff-a-11-0-UI.s1 Mus musculus cDNA, 3 end/clone = UI-M-AN1-aff-a-11-0-UI/ | 197.4 | P | 192.9 | P | 183.7 | P |
| | | clone_end = 3/gb = AI846484/gi = 5490390/ug = Mm.90698/len = 393/STRA = for | | | | | | |
| 5 | 102302_at | Cluster Incl L16992 Mus musculus branched-chain alpha-ketoacid dehydrogenase E1 beta-subunit mRNA sequence/ | 245 | P | 223.8 | P | 222.9 | P |
| | | cds = UNKNOWN/gb = L16992/gi = 293303/ug = Mm 12819/len = 1390/STRA = for | | | | | | |
| 5 | 102309_at | Cluster Incl AB012725.Mus musculus ZAN75 mRNA for zinc finger protein, complete cds/cds = (81,1823)/gb = AB012725/ | 94.3 | P | 87.5 | P | 77 | P |
| | | gi = 3298471/ug = Mm 12891/len = 2627/STRA = for | | | | | | |
| 5 | 102644_at | Cluster Incl U13371-Mus musculus (C57BL/10 X C3H)F2 clone 1 5 novel mRNA from renin-expressing kidney tumor cell | 241.1 | P | 222.6 | P | 247.8 | P |
| | | line, partial sequence/cds = UNKNOWN/gb = U13371/gi = 987223/ug = Mm 1314/len = 1217/STRA = for | | | | | | |
| 5 | 102710_at | Cluster Incl AF020313: Mus musculus proline-rich protein 48 mRNA, partial cds/cds = (0,895)/gb = AF020313/gi = 2624971/ | 43.3 | P | 103.7 | P | 33.9 | P |
| | | ug = Mm 14255/len = 2223/STRA = for | | | | | | |
| 5 | 103201_at | Cluster Incl M86377: Mouse esk kinase mRNA, complete cds/cds = (37,2607)/gb = M86377/gi = 193109/ug = Mm.1904/ | 141.4 | P | 101.7 | P | 94.2 | P |
| | | len = 2924/STRA = for | | | | | | |
| 5 | 103312_f_at | Cluster Incl AW047929 UI-M-BH1-aln-d-03-0-UI.s1 Mus musculus cDNA, 3 end/clone = UI-M-BH1-aln-d-03-0-UI/ | 285.6 | P | 503.2 | P | 323.6 | P |
| | | clone_end = 3/gb = AW047929/gi = 5908458/ug = Mm.21228/len = 461/STRA = rev | | | | | | |
| 5 | 103440_at | Cluster Incl AW226540: up08h09.x1 Mus musculus cDNA, 3 end/clone = IMAGE-2651489/clone_end = 3/gb = AW226540/ | 195.9 | P | 336.6 | P | 209.9 | P |
| | | gi = 6555836/ug = Mm 21424/len = 462/STRA = for | | | | | | |
| 5 | 103695_f_at | Cluster Incl AW047329 UI-M-BH1-amh-h-04-0-UI s1 Mus musculus cDNA, 3 end/clone = UI-M-BH1-amh-h-04-0-UI/ | 491 | P | 419.3 | P | 408.3 | P |
| | | clone_end = 3/gb = AW047329/gi = 5907858/ug = Mm 23994/len = 438/STRA = for | | | | | | |
| 5 | 103704_at | Cluster Incl AW047625: UI-M-BH1-akm-f-09-0-UI s1 Mus musculus cDNA, 3 end/clone = UI-M-BH1-akm-f-09-0-UI/ | 285 | P | 239.2 | P | 166.1 | P |
| | | clone_end = 3/gb = AW047625/gi = 5908154/ug = Mm.24075/len = 326/STRA = for | | | | | | |
| 5 | 103739_at | Cluster Incl AW230977 uo70h08 y1 Mus musculus cDNA, 5 end/clone = IMAGE-2647935/clone_end = 5/gb = AW230977/ | 295 | P | 304 | P | 299.4 | P |
| | | gi = 6560343/ug = Mm.24411/len = 424/STRA = for | | | | | | |
| 5 | 103753_at | Cluster Incl AI159572: vz77c07.r1 Mus musculus cDNA, 5 end/clone = IMAGE-1332492/clone_end = 5/gb = AI159572/ | 222.9 | P | 272 | P | 266.9 | P |
| | | gi = 3692754/ug = Mm.24527/len = 476/STRA = for | | | | | | |
| 5 | 103932_at | Cluster Incl AA655311: vq85f02.s1 Mus musculus cDNA, 5 end/clone = IMAGE-1109115/clone_end = 5/gb = AA655311/ | 351.6 | P | 344 | P | 464.9 | P |
| | | gi = 2591465/ug = Mm 25148/len = 416/STRA = for | | | | | | |
| 5 | 103947_at | Cluster Incl AW048038 UI-M-BH1-afv-c-02-0-UI s1 Mus musculus cDNA, 3 end/clone = UI-M-BH1-alv-c-02-0-UI/ | 199.9 | P | 267.2 | P | 136.5 | P |
| | | clone_end = 3/gb = AW048038/gi = 5908532/ug = Mm 25345/len = 269/STRA = for | | | | | | |
| 5 | 103969_at | Cluster Incl AA981154 vx57h02 r1 Mus musculus cDNA, 5 end/clone = IMAGE-1279347/clone_end = 5/gb = AA981154/ | 233.6 | P | 473.6 | P | 365 | P |
| | | gi = 3159690/ug = Mm 25715/len = 218/STRA = for | | | | | | |
| 5 | 104004_at | Cluster Incl AI931876: uI67b12.x1 Mus musculus cDNA, 3 end/clone = IMAGE-2135615/clone_end = 3/gb = AI931876/ | 265.6 | P | 359.7 | P | 257.1 | P |
| | | gi = 5668361/ug = Mm 26610/len = 529/STRA = for | | | | | | |
| 5 | 104015_at | Cluster Incl AW047992: UI-M-BH1-alv-e-08-0-UI s1 Mus musculus cDNA, 3 end/clone = UI-M-BHI-alv-e-08-0-UI/ | 275.7 | P | 503.6 | P | 348.6 | P |
| | | clone_end = 3/gb = AW047992/gi = 5908561/ug = Mm 26833/len = 478/STRA = for | | | | | | |
| 5 | 104412_at | Cluster Incl AI153412.uc53a01 r1 Mus musculus cDNA, 5 end/clone = IMAGE-1429320/clone_end = 5/gb = AI153412/ | 253.3 | P | 296.8 | P | 268.5 | P |
| | | gi = 3681881/ug = Mm 28859/len = 507/STRA = for | | | | | | |
| 5 | 104640_f_at | Cluster Incl AI464596: mz66c12 y1 Mus musculus cDNA, 5 end/clone = IMAGE-718390/clone_end = 5/gb = AI464598/ | 134.3 | P | 283.9 | P | 220.5 | P |
| | | gi = 4318626/ug = Mm.31114/len = 339/STRA = for | | | | | | |
| 5 | 92302_at | Cluster Incl Z11664 Son of sevenless homolog 2, (Drosophila)/cds = (0,3893)/gb = Z11664/gi = 54136/ug = Mm 3770/ | 70.6 | P | 139.5 | P | 130.3 | P |
| | | len = 5253/STRA = for | | | | | | |
| 5 | 92502_at | Cluster Incl X95504 M musculus mRNA for zinc finger protein/cds = UNKNOWN/gb = X95504/gi = 2828233/ug = Mm 42200/ | 201.1 | P | 192.2 | P | 109.9 | P |
| | | len = 3732/STRA = for | | | | | | |
| 5 | 92513_at | Cluster Incl AJ002636 Mus musculus mRNA for nuclear protein SA2/cds = (279,3767)/gb = AJ002638/gi = 2644956/ | 219.7 | P | 272.6 | P | 230.4 | P |
| | | ug = Mm 42238/len = 3871/STRA = for | | | | | | |
| 5 | 92660_f_at | Cluster Incl X92665 M. musculus mRNA for ubiquitin-conjugating enzyme UbcM3/cds = (153,734)/gb = X92665/gi = 107238/ | 402.3 | P | 426.3 | P | 402.3 | P |
| | | ug = Mm.4429/len = 1328/STRA = for | | | | | | |
| 5 | 93190_at | Cluster Incl AW209179 uo02h05.x1 Mus musculus cDNA, 3 end/clone = IMAGE-2582265/clone_end = 3/gb = AW209179/ | 234.3 | P | 269.8 | P | 194.6 | P |
| | | gi = 6515119/ug = Mm 5547/len = 428/STRA = for | | | | | | |
| 5 | 93877_at | Cluster Incl AI834777 UI-M-AM0-ads-c-04-0-UI s1 Mus musculus cDNA, 3 end/clone = UI-M-AM0-ads-c-04-0-UI/ | 350.1 | P | 517.7 | P | 317 | P |
| | | clone_end = 3/gb = AI834777/gi = 5469010/ug = Mm 886/len = 279/STRA = for | | | | | | |
| 5 | 94732_at | Cluster Incl X99251: Repetin/cds = (65,3457)/gb = X99251/gi = 1806131/ug = Mm 1417/len = 4178/STRA = for | 115.7 | P | 724.9 | P | 738.1 | P |
| 5 | 94768_at | Cluster Incl D37790 Mouse mRNA for beta-1,4-galactosyltransferase/cds = UNKNOWN/gb = D37790/gi = 517126/ | 1506.2 | P | 1464.7 | P | 1193.9 | P |
| | | ug = Mm 164/len = 3298/STRA = rev | | | | | | |
| 5 | 95395_at | Cluster Incl AI225869 uI22e07 y1 Mus musculus cDNA, 5 end/clone = IMAGE-1852068/clone_end = 5/gb = AI225869/ | 125.9 | P | 200.2 | P | 124.4 | P |
| | | gi = 3808922/ug = Mm 23834/len = 388/STRA = for | | | | | | |
| 5 | 97689_at | Cluster Incl M26071: Coagulation factor III/cds = UNKNOWN/gb = M26071/gi = 201924/ug = Mm 3742/len = 1821/STRA = for | 329.6 | P | 403.5 | P | 315.7 | P |
| 5 | 95787_s_at | Cluster Incl X87685 M musculus sterol camer protein 2-pseudogene/cds = (0,478)/gb = X87685/gi = 854333/ug = Mm 87814/ | 276.8 | P | 419.1 | P | 292 | P |
| | | len = 479/STRA = for | | | | | | |
| 5 | 92542_at | Cluster Incl AI849035 UI-M-AH1-agw-a-06-0-UI s1 Mus musculus cDNA, 3 end/clone = UI-M-AH1-agw-a-06-0-UI/ | 170.1 | P | 458.3 | P | 302.1 | P |
| | | clone_end = 3/gb = AI849035/gi = 5492941/ug = Mm.10009/len = 377/STRA = for | | | | | | |
| 5 | 92579_at | Cluster Incl L00993 Sjogren syndrome antigen B/cds = (140,1387)/gb = L00993/gi = 198712/ug = Mm.10508/len = 1961/ | 99 | P | 120.6 | P | 44 | A |
| | | STRA = for | | | | | | |
| 5 | 92648_at | Cluster Incl U19521: Mus musculus vesicle transport protein (munc.18c) mRNA, complete cds/cds = (159,1937)/gb = U19521/ | 139.3 | P | 210.7 | P | 151.1 | P |
| | | gi = 642027/ug = Mm 12155/len = 2037/STRA = for | | | | | | |
| 5 | 93253_at | Cluster Incl D87271: Mus musculus DNA for ERK2/cds = (223,1098)/gb = D87271/gi = 2506074/ug = Mm 1700/len = 1182/ | 128.1 | P | 132.8 | P | 107.1 | P |
| | | STRA = for | | | | | | |
| 5 | 95438_at | Cluster Incl AI846783 UI-M-AO1-ael-f-12-0-UI s1 Mus musculus cDNA, 3 end/clone = UI-M-AO1-ael-f-12-0-UI/ | 108.4 | P | 162.1 | P | 164 | P |
| | | clone_end = 3/gb = AI846783/gi = 5490689/ug = Mm 24541/len = 269/STRA = for | | | | | | |
| 5 | 96616_at | Cluster Incl AI891629 ul57e11.x1 Mus musculus cDNA, 3 end/clone = IMAGE-2123180/clone_end = 3/gb = AI891629/ | 124 | P | 266.2 | P | 214.5 | P |
| | | gi = 5597531/ug = Mm 27123/len = 443/STRA = for | | | | | | |
| 5 | 96266_at | Cluster Incl AI841982 UI-M-AHO-acu-h-04-0-UI s2 Mus musculus cDNA, 3 end/clone = UI-M-AH0-acu-h-04-0-UI/ | 268.5 | P | 706 | P | 326.4 | A |
| | | clone_end = 3/gb = AI841982/gi = 5476195/ug = Mm 29841/len = 334/STRA = for | | | | | | |
| 5 | 99194_at | Cluster Incl AW124271 UI-M-BH2 1-aph-h-11-0-UI s1 Mus musculus cDNA, 3 end/clone = UI-M-BH2.1-aph-h-11-0-UI/ | 70.8 | P | 119.8 | P | 93 | P |
| | | clone_end = 3/gb = AW124271/gi = 6099801/ug = Mm 41935/len = 363/STRA = for | | | | | | |
| 5 | 101475_at | Cluster Incl M64068 Mouse zinc finger protein (bmi-1) gene, complete cds/cds = (0,749)/gb = M64068/gi = 192204/ | 105.1 | A | 175.8 | P | 270 | P |
| | | ug = Mm.7719/len = 750/STRA = for | | | | | | |
| 5 | 101965_at | Cluster Incl AF037205 Mus musculus RING zinc finger protein (Rzf) mRNA, complete cds/cds = (88,1233)/gb = AF037205/ | 223.1 | A | 350.7 | P | 196.5 | P |
| | | gi = 2746334/ug = Mm.9326/len = 2240/STRA = for | | | | | | |
| 5 | 103083_at | Cluster Incl U69543 Lipase, hormone sensitive/cds = (592,2871)/gb = U69543/gi = 1899053/ug = Mm 1721/len = 3175/ | 154.8 | A | 392.4 | P | 460.3 | P |
| | | STRA = for | | | | | | |
| 5 | 103328_at | Cluster Incl U59864 Mus musculus TRAF-interacting protein I-TRAF mRNA, complete cds/cds = (68,1414)/gb = U59864/ | 81 | A | 99.5 | P | 69.1 | P |
| | | gi = 1518019/ug = Mm 1803/len = 1971/STRA = for | | | | | | |
| 5 | 103596_at | Cluster Incl AF085219: Mus musculus alpha diacylglycerol kinase mRNA, complete cds/cds = (19,2211)/gb = AF085219/ | 46.9 | M | 124.2 | P | 50.2 | P |
| | | gi = 3493665/ug = Mm 18981/len = 2239/STRA = for | | | | | | |
| 5 | 103638_at | Cluster Incl AW046460 UI-M-BH1-akp-g-06-0-UI s1 Mus musculus cDNA, 3 end/clone = UI-M-BH1-akp-g-06-0-UI/ | 66.4 | P | 105.9 | P | 49.7 | P |
| | | clone_end = 3/gb = AW046460/gi = 5906989/ug = Mm.2035/len = 278/STRA = for | | | | | | |
| 5 | 104060_at | Cluster Incl AI845977 UI-M-AP1-agi-b-12-0-UI s1 Mus musculus cDNA, 3 end/clone = UI-M-AP1-agl-b-12-0-UI/ | 138.9 | P | 218.3 | P | 175.2 | P |
| | | clone_end = 3/gb = AI845977/gi = 5489883/ug = Mm 21568/len = 351/STRA = for | | | | | | |
| 5 | 104070_at | Cluster Incl AW047728 UI-M-BH1-alk-a-01-0-UI s1 Mus musculus cDNA, 3 end/clone = UI-M-BH1-alk-a-01-0-UI/ | 56.8 | M | 199.2 | P | 142.9 | P |
| | | clone_end = 3/gb = AW047728/gi = 5908257/ug = Mm 21637/len = 392/STRA = for | | | | | | |
| 5 | 104290_at | Cluster Incl AI847837 UI-M-AM1-afu-b-08-o-UI.s1 Mus musculus cDNA, 3 end/clone = UI-M-AM1-afu-b-08-0-UI/ | 52.8 | A | 117.2 | P | 91.4 | P |
| | | clone_end = 3/gb = AI847837/gi = 5491743/ug = Mm 22279/len = 387/STRA = rev | | | | | | |
| 5 | 104558_at | Cluster Incl AA867123: vx23f05.r1 Mus musculus cDNA, 5 end/clone = IMAGE-1265313/clone_end = 5/gb = AA867123/ | 300.3 | A | 116.2 | P | 236.9 | P |
| | | gi = 2962568/ug = Mm.23808/len = 552/STRA = for | | | | | | |
| 5 | 104706_at | Cluster Incl U69171: Mus musculus peroxisomal PTS2 receptor mRNA, complete cds/cds = (92,1048)/gb = U69171/ | 44.8 | P | 62.2 | P | 87.5 | P |
| | | gi = 1890656/ug = Mm 2440/len = 1468/STRA = for | | | | | | |
| 5 | 94923_f_at | Cluster Incl AA543502 vk25c02 r1 Mus musculus cDNA, 5 end/clone = IMAGE-947618/clone_end = 5/gb = AA543502/ | 291.3 | A | 616.8 | P | 652.3 | P |
| | | gi = 2291979/ug = Mm 26609/len = 598/STRA = rev | | | | | | |
| 5 | 94992_at | Cluster Incl AI840667 UI-M-AM0-adr-b-05-0-UI s1 Mus musculus cDNA, 3 end/clone = UI-M-AM0-adr-b-05-0-UI/ | 176.3 | M | 290.9 | P | 292.6 | P |
| | | clone_end = 3/gb = AI840667/gi = 5474875/ug = Mm 27327/len = 304/STRA = for | | | | | | |
| 5 | 95040_at | Cluster Incl AI840810 UI-M-AH0-adb-b-09-0-UI s1 Mus musculus cDNA, 3 end/clone = UI-M-AH0-adb-b-09-0-UI/ | 88.3 | P | 139.2 | P | 86.9 | M |
| | | clone_end = 3/gb = AI840810/gi = 5475023/ug = Mm 27569/len = 347/STRA = for | | | | | | |
| 5 | 96215_f_at | Cluster Incl AI153421 uc53a11.r1 Mus musculus cDNA, 5 end/clone = IMAGE-1429340/clone_end = 5/gb = AI153421/ | 535.6 | P | 509.2 | P | 505.8 | P |
| | | gi = 3681890/ug = Mm 28674/len = 385/STRA = for | | | | | | |
| 5 | 97422_at | Cluster Incl AW048882 UI-M-BH1-amf-e-12-0-UI s1 Mus musculus cDNA, 3 end/clone = UI-M-BH1-amf-e-12-0-UI/ | 96.7 | P | 202.4 | P | 178.9 | P |
| | | clone_end = 3/gb = AW048882/gi = 5909411/ug = Mm 29999/len = 497/STRA = for | | | | | | |
| 5 | 97976_at | Cluster Incl L43326.Kinectin 1/cds = (19,4002)/gb = L43326/gi = 887379/ug = Mm 3110/len = 4466/STRA = rev | 62.7 | P | 183.2 | P | 72.1 | A |
| 5 | 103727_at | Cluster Incl AI850972 UI-M-BH0-akd-g-07-0-UI s1 Mus musculus cDNA, 3 end/clone = UI-M-BH0-akd-g-07-0-UI/ | 23.6 | P | 117.1 | P | 129.2 | P |
| | | clone_end = 3/gb = AI850972/gi = 5494878/ug = Mm 24291/len = 370/STRA = for | | | | | | |
| 5 | 104193_at | Cluster Incl AW047583 UI-M-BH1-akm-b-04-0-UI a1Mus musculus cDNA, 3 end/clone = UI-M-BH1-akm-b-04-0-UI/ | 72.2 | A | 221.1 | P | 193.2 | P |
| | | clone_end = 3/gb = AW047583/gi = 5908112/ug = Mm 27493/len = 426/STRA = for | | | | | | |
| 5 | 104683_at | Cluster Incl AW120783 UI-M-BH2.3-any-g-06-0-UI.s1 Mus musculus cDNA, 3 end/clone = UI-M-BH2.3-any-g-06-0-UI/ | 108.4 | A | 227.8 | P | 190.4 | P |
| | | clone_end = 3/gb = AW120783/gi = 6096116/ug = Mm.3289/len = 422/STRA = for | | | | | | |
| 5 | 93203_f_at | Cluster Incl AW107463 um17a12x1 Mus musculus cDNA, 3 end/clone = IMAGE-2192542/clone_end = 3/gb = AW107463/ | 93.4 | P | 104.3 | P | 127.2 | P |
| | | gi = 6078263/ug = Mm.5697/len = 484/STRA = rev | | | | | | |
| 5 | 102130_f_at | Cluster Incl C88243 C88243 Mus musculus cDNA, 3 end/clone = J0259A06/clone_end = 3/gb = C88243/gi = 2920200/ | 192.6 | P | 189.1 | P | 129.8 | P |
| | | ug = Mm 43522/len = 610/STRA = rev | | | | | | |
| 5 | 96608_at | Cluster Incl AF023463 Mus musculus peroxisomal phytanoyl-CoA alpha-hydroxylase (PAHX) mRNA, complete cds/ | 90.4 | M | 146.7 | P | 109.6 | P |
| | | cds = (39,1055)/gb = AF023463/gi = 2564672/ug = Mm 27066/len = 1419/STRA = for | | | | | | |
| 5 | 97853_at | Cluster Incl AW047331.UI-M-BH1-amh-h-07-0-UI s1 Mus musculus cDNA, 3 end/clone = UI-M-BH1-amh-h-07-0-UI/ | 46.5 | M | 108.8 | P | 65.1 | P |
| | | clone_end = 3/gb = AW047331/gi = 5907860/ug = Mm 28287/len = 333/STRA = for | | | | | | |
| 5 | 97869_at | Cluster Incl AI844043 UI-M-AL1-ahi-d-11-0-UI s1 Mus musculus cDNA, 3 end/clone = UI-M-AL1-ahi-d-11-0-UI/ | 70.4 | A | 146.9 | P | 137.3 | P |
| | | clone_end = 3/gb = AI844043/gi = 5487949/ug = Mm.28336/len = 378/STRA = for | | | | | | |
| 5 | 97892_at | Cluster Incl AI844732 UI-M-AL1-ahq-c-04-0-UI s1 Mus musculus cDNA, 3 end/clone = UI-M-AL1-ahq-c-04-0-UI/ | 43.3 | P | 52.8 | P | 52.2 | P |
| | | clone_end = 3/gb = AI844732/gi = 5488638/ug = Mm 28414/len = 475/STRA = for | | | | | | |
| 5 | 96850_at | Cluster Incl AI853842 UI-M-BH0-ajq-a-04-0-UI.s1 Mus musculus cDNA, 3 end/clone = UI-M-BH0-ajq-a-04-0-UI/ | 52.4 | A | 165.3 | P | 154.3 | P |
| | | clone_end = 3/gb = AI853642/gi = 5497546/ug = Mm 30036/len = 511/STRA = for | | | | | | |
| 5 | 98071_f_at | Cluster Incl X77731 M. musculus mRNA for Deoxycytidine kinase/cds = (12,794)/gb = X7731/gi = 456676/ug = Mm 3446/ | 349.2 | P | 247.8 | A | 265.1 | P |
| | | len = 2812/STRA = for | | | | | | |
| 5 | 101498_at | Cluster Incl AF042730 Mus musculus lithium-sensitive myo-inositol monophosphatase A1 (IMPA1) mRNA, complete cds/ | 59.8 | P | 133.9 | P | 73.2 | P |
| | | cds = (66,899)/gb = AF042730/gi = 2801800/ug = Mm 8089/len = 2167/STRA = for | | | | | | |
| 5 | 101996_at | Cluster Incl M80739 1570 bp/cds = UNKNOWN/gb = M80739/gi = 485375/ug = Mm 985/len = 1570/STRA = for | 84.2 | A | 136.4 | P | 108.7 | P |
| 5 | 102318_at | Cluster Incl X86000 Mus musculus mRNA for N-glycan alpha 2,8-sialyltransferase/cds = (333,1412)/gb = X86000/ | 58.2 | P | 53.2 | A | 84.4 | P |
| | | gi = 2685331/ug = Mm 10701/len = 6786/STRA = for | | | | | | |
| 5 | 102321_at | Cluster Incl M93422 Adenylate cyclase 6/cds = (95,3592)/gb = M93422/gi = M93422/gi = 191690/ug = Mm 10706/ | 201.1 | P | 131 | P | 242.5 | P |
| | | len = 5841/STRA = for | | | | | | |
| 5 | 104316_at | Cluster Incl AI646098: vw54h09 x1 Mus musculus cDNA, 3 end/clone = IMAGE-1247681/clone_end = 3/gb = AI646098/ | 75.2 | A | 227.7 | P | 164.8 | P |
| | | gi = 4724573/ug = Mm 22414/len = 501/STRA = rev | | | | | | |
| 5 | 94397_at | Cluster Incl AA690091: vr79a09 s1 Mus musculus cDNA, 5 end/clone = IMAGE-1134904/clone_end = 5/gb = AA690091/ | 181.8 | P | 153 | P | 129.6 | P |
| | | gi = 2691027/ug = Mm 25776/len = 347/STRA = for | | | | | | |
| 5 | 94979_at | Cluster Incl AW121624: UI-M-BH2 2-acq-g-11-0-UI.s1 Mus musculus cDNA, 3 end/clone = UI-M-BH2.2-aoq-g-11-0-UI/ | 22.8 | P | −7.9 | P | −9.1 | M |
| | | clone_end = 3/gb = AW121624/gi = 6096957/ug = Mm 27273/len = 334/STRA = for | | | | | | |
| 5 | 95001_at | Cluster Incl AB028920 Mus musculus AKAP95 mRNA for A kinase anchor protein 95, complete cds/cds = (38,2101)/ | 118.8 | P | 171.7 | P | 116.5 | P |
| | | gb = AB028920/gi = 5931617/ug = Mm 27351/len = 2704/STRA = for | | | | | | |
| 5 | 95020_at | Cluster Incl AI848868 UI-M-AJ1-ahb-f-03-0-UI s1 Mus musculus cDNA, 3 end/clone = UI-M-AJ1-ahb-f-03-0-UI/ | 115.7 | P | 218.9 | P | 159.7 | A |
| | | clone_end = 3/gb = AI848868/gi = 5492774/ug = Mm 27466/len = 496/STRA = for | | | | | | |
| 5 | 95028_r_at | Cluster Incl C77009 C77009 Mus musculus cDNA, 3 end/clone = J0024D07/clone_end = 3/gb = C77009/gi = 2517339/ | 154.3 | P | 378.9 | P | 328.2 | P |
| | | ug = Mm 27511/len = 496/STRA = rev | | | | | | |
| 5 | 97919_at | Cluster Incl AI837467 UI-M-AL0-abr-d-06-0-UI s1 Mus musculus cDNA,3 end/clone = UI-M-AL0-abr-d-06-0-UI/ | 25.6 | A | 112.6 | P | 75.8 | P |
| | | clone_end = 3/gb = AI837467/gi = 5471680/ug = Mm 30132/len = 228/STRA = for | | | | | | |
| 5 | 98504_at | Cluster Incl U58513 Mus musculus Rho-associated, coiled-coil forming protein kinase p160 ROCK-2 mRNA, complete cds/ | 94.8 | P | 137.6 | P | 45.8 | M |
| | | cds = (0,4166)/gb = U58513/gi = 1514697/ug = Mm 35815/len = 4167/STRA = for | | | | | | |
| 5 | 100894_at | Cluster Incl AJ007360 Mus musculus mRNA for ORC5-related protein/cds = (65,1372)/gb = AJ007360/gi = 4938333/ | 73.3 | P | 70.3 | A | 75.7 | P |
| | | ug = Mm 566/len = 2079/STRA = for | | | | | | |
| 5 | 103202_at | Cluster Incl AW047476.UI-M-BH1-all-g-04-0-UI s1 Mus musculus cDNA, 3 end/clone = UI-M-BH1-all-g-04-0-UI/ | 105.8 | P | 264.1 | P | 365.5 | P |
| | | clone_end = 3/gb = AW047476/gi = 5908005/ug = Mm 1909/len = 248/STRA = for | | | | | | |
| 5 | 103207_at | Cluster Incl D13543 DNA polymerase alpha 1, 180 kDa/cds = (21,4418)/gb = D13543/gi = 475158/ug = Mm.1923/len = 5323/ | 93.3 | P | 108 | P | 73.7 | P |
| | | STRA = for | | | | | | |
| 5 | 92226_at | Cluster Incl AA866971 vx91g10.r1 Mus musculus cDNA, 5 end/clone = IMAGE-1282626/clone_end = 5/gb = AA866971/ | 129.4 | M | 139.7 | A | 204.1 | P |
| | | gi = 2962416/ug = Mm.346/len = 370/STRA = for | | | | | | |
| 5 | 96526_at | Cluster Incl AW228840 up22f10.x1 Mus musculus cDNA, 3 end/clone = IMAGE-2655115/clone_end = 3/gb = AW228840/ | 129.3 | A | 242.7 | P | 161.1 | P |
| | | gi = 6558136/ug = Mm.2670/len = 133/STRA = for | | | | | | |
| 5 | 98893_at | Cluster Incl AW048644 UI-M-BH1-amd-d-08-0-UI s1 Mus musculus cDNA, 3 end/clone = UI-M-BH1-amd-d-08-0-UI/ | 82.3 | P | 256.6 | P | 257.5 | P |
| | | clone_end = 3/gb = AW048644/gi = 5909173/ug = Mm 28550/len = 430/STRA = for | | | | | | |
| 5 | 103010_at | Cluster Incl AW048174: UI-M-BH1-amc-b-12-0-UI.s1 Mus musculus cDNA, 3 end/clone = UI-M-BH1-amc-b-12-0-UI/ | 151.7 | P | 198 | P | 89.4 | P |
| | | clone _end = 3/gb = AW048174/gi = 5908703/ug = Mm.1571/len = 398/STRA = for | | | | | | |
| 5 | 103076_at | Cluster Incl AW046093 UI-M-BH1-alf-a-06-0-UI s1 Mus musculus cDNA, 3 end/clone = UI-M-BH1-alf-a-06-0-UI/ | 293.8 | A | 358.6 | P | 313.9 | A |
| | | clone _end = 3/gb = AW046093/gi = 5906622/ug = Mm 17087/len = 258/STRA = for | | | | | | |
| 5 | 103422_at | Cluster Incl M63695 CD1d1 antigen/cds = (91,1101)/gb = M63695/gi = 192471/ug = Mm 1894/len = 1838/STRA = for | 23.5 | A | 40.6 | P | 55.3 | P |
| 5 | 103895_at | Cluster Incl AW121745: UI-M-BH2 3-aoe-c-07-0-UI s1 Mus musculus cDNA, 3 end/clone = UI-M-BH2 3-aoe-c-07-0-UI/ | 13.6 | A | 68.3 | P | 17.7 | P |
| | | clone _end = 3/gb = AW121745/gi = 6097080/ug = Mm 21299/len = 345/STRA = for | | | | | | |
| 5 | 104049_at | Cluster Incl AW047134: UI-M-BH1-alu-c-03-0-UI s1 Mus musculus cDNA, 3 end/clone = UI-M-BH1-alu-c-03-0-UI/ | 18.6 | A | 89.1 | P | 59.7 | P |
| | | clone _end = 3/gb = AW047134/gi = 5907663/ug = Mm 21510/len = 332/STRA = for | | | | | | |
| 5 | 94934_at | Cluster Incl U04828: Angiotensin II receptor, type 2/cds = (131,1222)/gb = U04828/gi = 439862/ug = Mm.2679/len = 2827/ | 399.8 | P | 116 | A | 291.5 | P |
| | | STRA = for | | | | | | |
| 5 | 96741_at | Cluster Incl AW124129: UI-M-BH2 1-apr-d-08-0-UI s1 Mus musculus cDNA, 3 end/clone = UI-M-BH2 1-apr-d-08-0-UI/ | 9.9 | P | 59.2 | P | 29.5 | P |
| | | clone _end = 3/gb = AW124129/gi = 6099659/ug = Mm 28932/len = 192/STRA = rev | | | | | | |
| 5 | 99999_at | Cluster Incl D83033 Mouse mRNA for nuclear protein, NP220, complete cds/cds = (325,6105)/gb = D83033/gi = 1469808/ | 57.3 | A | 149.3 | P | 90 | A |
| | | ug = Mm 4503/len = 6345/STRA = for | | | | | | |
| 5 | 100903_at | Cluster Incl AJ007780 Mus musculus mRNA for poly(ADP-ribose) polymerase-2/cds = (15,1694)/gb = AJ007780/ | 73.7 | P | 69.4 | A | 31 | P |
| | | gi = 4138232/ug = Mm 5728/len = 1707/STRA = for | | | | | | |
| 5 | 104267_at | Cluster Incl AI844736: UI-M-AL1-ahq-c-10-0-UI s1 Mus musculus cDNA, 3 end/clone = UI-M-AL1-ahq-c-10-0-UI/ | 277.1 | P | 295.3 | P | 277.4 | P |
| | | clone _end = 3/gb = AI844736/gi = 5488642/ug = Mm 28535/len = 406/STRA = rev | | | | | | |
| 5 | 93887_at | Cluster Incl AI854351 UI-M-BG1-aic-d-05-0-UI s1 Mus musculus cDNA, 3 end/clone = UI-M-BG1-aic-d-05-0-UI/ | 92 | P | 134.9 | A | 91.5 | P |
| | | clone _end = 3/gb = AI854351/gi = 5498257/ug = Mm 88860/len = 369/STRA = for | | | | | | |
| 5 | 94282_at | Cluster Incl AW124297 UI-M-BH2 1-apq-c-02-0-UI s1 Mus musculus cDNA, 3 end/clone = UI-M-BH2 1-apq-c-02-0-UI/ | 171.9 | A | 268.2 | A | 150.7 | P |
| | | clone _end = 3/gb = AW124297/gi = 6099827/ug = Mm 22547/len = 237/STRA = for | | | | | | |
| 5 | 96945_at | Cluster Incl U73143 Mus musculus syndet mRNA, complete cds/cds = (0,632)/gb = U73143/gi = 2065524/ug = Mm.30197/ | 96.8 | P | 120.9 | P | 67.3 | A |
| | | len = 633/STRA = for | | | | | | |
| 5 | 102388_at | Cluster Incl AW121186: UI-M-BH2 3-aok-b-02-0-UI s1 Mus musculus cDNA, 3 end/clone = UI-M-BH2 3-aok-b-02-0-UI/ | 94.2 | P | 115.8 | A | 130 | A |
| | | clone _end = 3/gb = AW121186/gi = 6096519/ug = Mm 1182/len = 501/STRA = for | | | | | | |
| 5 | 103813_at | Cluster Incl D85926: Mus musculus mRNA for Sh3yl1, complete cds/cds = (41,1063)/gb = D85926/gi = 1944388/ | 60.7 | P | 68.6 | P | 54.8 | A |
| | | ug = Mm 2090/len = 1226/STRA = for | | | | | | |
| 5 | 104119_at | Cluster Incl AI845028: UI-M-BGO-ahy-a-01-0-UI s1 Mus musculus cDNA, 3 end/clone = UI-M-BGO-ahy-a-01-0-UI/ | 71.2 | A | 116 | P | 57.4 | P |
| | | clone_end = 3/gb = AI845028/gi = 5488934/ug = Mm.21977/len = 510/STRA = for | | | | | | |
| 5 | 104380_at | Cluster Incl Z71268: M musculus mRNA for CMP-stalic acid transporter/cds = (0,1010)/gb = Z71268/gi = 1486407/ | 51 | A | 94.4 | A | 81.7 | P |
| | | ug = Mm 22684/len = 1185/STRA = for | | | | | | |
| 5 | 104759_at | Cluster Incl AI851421: UI-M-BH0-akj-c-04-0-UI s1 Mus musculus cDNA, 3 end/clone = UI-M-BH0-akj-c-04-0-UI/ | 206.3 | P | 195.8 | P | 118.8 | P |
| | | clone_end = 3/gb = AI851421/gi = 5495327/ug = Mm 24797/len = 337/STRA = for | | | | | | |
| 5 | 94386_at | Cluster Incl AI850140 UI-M-BG1-aid-c-11-0-UI s1 Mus musculus cDNA, 3 end/clone = UI-M-BG1-aid-c-11-0-UI/ | 131 | A | 271.6 | P | 230.2 | A |
| | | clone_end = 3/gb = AI850140/gi = 5494046/ug = Mm 25622/len = 305/STRA = for | | | | | | |
| 5 | 95549_at | Cluster Incl D13545 DNA primase, p58 subunit/cds = (144,1661)/gb = D13545/gi = 303662/ug = Mm 27705/len = 1937/ | 74.9 | P | 27.4 | A | 70.1 | A |
| | | STRA = for | | | | | | |
| 5 | 96746_at | Cluster Incl AW124813: UI-M-BH2.1-apk-c-01-0-UI.s1 Mus musculus cDNA, 3 end/clone = UI-M-BH2.1-apk-c-01-0-UI/ | 84.4 | A | 105.5 | A | 119.5 | P |
| | | clone_end = 3/gb = AW124813/gi = 6100343/ug = Mm 28953/len = 435/STRA = for | | | | | | |
| 5 | 96785_at | Cluster Incl AF110520 Mus musculus major histocompatibility complex region NG27, NG28, RPS28, NADH | 133.7 | P | 66.1 | A | 85.1 | P |
| | | oxidoreductase NG29, KIFC1, Fas-binding protein, BING1, tapasin, RaIGDS-like, KE2, BING4, beta 1,3-galactosyl | | | | | | |
| | | transferase, and RPS18 genes, complete cds, S/STRA = for | | | | | | |
| 5 | 99441_at | Cluster Incl AW123852 UI-M-BH2 1-app-b-06-0-UI.s1 Mus musculus cDNA, 3 end/clone = UI-M-BH2.1-app-b-06-0-UI/ | 34.9 | A | 19.5 | A | 65.9 | P |
| | | clone_end = 3/gb = AW123852/gi = 6099382/ug = Mm 40282/len = 451/STRA = for | | | | | | |
| 5 | 99963_at | Cluster Incl U07861: Zinc finger protein 101/cds = (107,1927)/gb = U07861/gi = 466390/ug = Mm 4417/len = 2177/STRA = rev | −56.4 | A | 34.5 | P | −10.8 | A |
| 5 | 100880_at | Cluster Incl AA816121 vp44a01.r1 Mus musculus cDNA, 5 end/clone = IMAGE-1079496/clone_end = 5/gb = AA816121/ | 139.2 | A | 208 | A | 266.2 | P |
| | | gi = 2885717/ug = Mm.532/len = 316/STRA = rev | | | | | | |
| 5 | 102957_at | Cluster Incl U20159: Mus musculus 78 kDa tyrosine phosphoprotein SLP-76 mRNA, complete cds/cds = (192,1793)/ | 6.1 | A | 56.6 | P | 47.2 | A |
| | | gb = U20159/gi = 806767/ug = Mm 1781/len = 2247/STRA = for | | | | | | |
| 5 | 103537_at | Cluster Incl AF076681: Mus musculus type-I transmembrane ER-resident serine/threonine kinase PERK mRNA, complete | 5.2 | A | 26.5 | P | 26.1 | P |
| | | cds/cds = (191,3535)/gb = AF076681/gi = 4107512/ug = Mm 23375/len = 4510/STRA = for | | | | | | |
| 5 | 104670_at | Cluster Incl AI315698 uj34a01 x1 Mus musculus cDNA, 3 end/clone = IMAGE-1921800/clone_end = 3/gb = AI315698/ | 64.6 | P | 39.9 | A | 81.1 | A |
| | | gi = 4030965/ug = Mm 32357/len = 215/STRA = for | | | | | | |
| 5 | 93241_r_at | Cluster Incl AI838094 UI-M-AL0-abv-g-07-0-UI s1 Mus musculus cDNA, 3 end/clone = UI-M-AL0-abv-g-07-0-UI/ | 35.4 | A | 64.6 | P | 31.2 | A |
| | | clone _end = 3/gb = AI838094/gi = 5472307/ug = Mm 5935/len = 344/STRA = rev | | | | | | |
| 5 | 94088_at | Cluster Incl AW228429 up22e01 y1 Mus musculus cDNA, 5 end/clone = IMAGE-2655096/clone _end = 5/gb = AW228429/ | 67.7 | P | 104.1 | P | 67.2 | A |
| | | gi = 6557725/ug = Mm 99371/len = 342/STRA = for | | | | | | |
| 5 | 95288_i_at | Cluster Incl AA189811 mt97g08.r1 Mus musculus cDNA, 5 end/clone = IMAGE-637886/clone_end = 5/gb = AA189811/ | 16.8 | A | 45.7 | P | 11.3 | A |
| | | gi = 1776416/ug = Mm 1882/len = 365/STRA = for | | | | | | |
| 5 | 95939_i_at | Cluster Incl AW047237: UI-M-BH1-amb-g-05-0-UI.s1 Mus musculus cDNA, 3 end/clone = UI-M-BH1-amb-g-05-0-UI/ | 114.1 | A | 126.1 | P | 54.4 | P |
| | | clone_end = 3/gb = AW047237/gi = 5907766/ug = Mm 24851/len = 420/STRA = for | | | | | | |
| 5 | 93538_at | Cluster Incl AW228036: Up12e07.x1 Mus musculus cDNA, 3 end/clone = IMAGE-2654148/clone_end = 3/gb = AW228036/ | 47.3 | M | 86.4 | P | 44.8 | A |
| | | gi = 6557332/ug = Mm 19992/len = 516/STRA = for | | | | | | |
| 5 | 94812_at | Cluster Incl AA684175 vm68b10. s1 Mus musculus cDNA, 5 end/clone = IMAGE-1003387/clone_end = 5/gb = AA684175/ | 87.4 | A | 189.7 | P | 27 | A |
| | | gi = 2670761/ug = Mm 22700/len = 513/STRA = for | | | | | | |
| 5 | 94818_at | Cluster Incl AW047223: UI-M-BH1-amb-e-09-0-UI.s1 Mus musculus cDNA, 3 end/clone = UI-M-BH1-amb-c-09-0-UI/ | 66.3 | A | 120.4 | A | 116.4 | M |
| | | clone_end = 3/gb = AW047223/gi = 5907752/ug = Mm 22710/len = 420/STRA = for | | | | | | |
| 5 | 98048_at | Cluster Incl AF080490 Mus musculus TLS-associated protein TASR-2 mRNA, complete cds/cds = (91,879)/gb = AF060490/ | 13.1 | A | 63.8 | A | 66.8 | A |
| | | gi = 3327956/ug = Mm 34279/len = 2693/STRA = for | | | | | | |
| 5 | 98543_at | Cluster Incl AJ223208: Mus musculus mRNA for cathepsin S, partial/cds = (0,139)/gb = AJ223208/gi = 4138249/ | −3.7 | A | 84.6 | A | −8.6 | A |
| | | ug = Mm.3619/len = 366/STRA = for | | | | | | |
| 5 | 99178_at | Cluster Incl AI845652: UI-M-AQ1-ady-b-04-0-UI.a1 Mus musculus cDNA, 3 end/clone = UI-M-AQ1-ady-b-04-0-UI/ | −2.2 | A | 53 | A | 38.3 | P |
| | | clone_end = /gb = AI845652/gi = 5489558/ug = Mm.41791/len = 357/STRA = for | | | | | | |
| 5 | 101082_at | Cluster Incl J02652: Malic enzyme, supernatant/cds = (65,1783)/gb = J02652/gi = 199762/ug = Mm 730/len = 3129/STRA = for | 276.3 | A | 285.9 | A | 305.8 | P |
| 5 | 94395_at | Cluster Incl AI194254 ul14a10.r1 Mus musculus cDNA, 5 end/clone = IMAGE-1851258/clone_end = 5/gb = AI194254/ | −2 | A | 40.4 | P | 1.1 | A |
| | | gi = 3745461/ug = Mm 25703/len = 475/STRA = for | | | | | | |
| 5 | 96231_at | Cluster Incl AW123780 UI-M-BH2 1-apm-c-10-0-UI s1 Mus musculus cDNA, 3 end/clone = UI-M-BH2.1-apm-c-10-0-UI/ | 0.9 | A | 114.2 | A | 118.4 | P |
| | | clone_end = 3/gb = AW123780/gi = 6099310/ug = Mm 28775/len = 400/STRA = for | | | | | | |
| 5 | 96778_at | Cluster Incl AA656775 vr50e03 s1 Mus musculus cDNA, 5 end/clone = IMAGE-1124092/clone_end = 5/gb = AA656775/ | 69.9 | A | 220.4 | A | 199.3 | P |
| | | gi = 2592929/ug = Mm 29061/len = 417/STRA = for | | | | | | |
| 5 | 98026_g_at | Cluster Incl M34896: Ecotropic viral integration site 2/cds = (191,862)/gb = M34896/gi = 193202/ug = Mm.3266/len = 1217/ | 168.6 | A | 156.1 | A | 145.5 | P |
| | | STRA = for | | | | | | |
| 5 | 98972_at | Cluster Incl AI574262 uj19e05 x1 Mus musculus cDNA, 3 end/clone = IMAGE-1920416/clone_end = 3/gb = AI574262/ | −137.7 | P | −57.9 | P | 2.5 | P |
| | | gi = 4537636/ug = Mm 36569/len = 474/STRA = for | | | | | | |
| 5 | 104263_at | Cluster Incl AA914734 vy91g08.r1 Mus musculus cDNA, 5 end/clone = IMAGE-1313630/clone_end = 5/gb = AA914734/ | 40.7 | A | 68.1 | P | 37.7 | A |
| | | gi = 3054126/ug = Mm 28450/len = 470/STRA = rev | | | | | | |
| 5 | 104274_at | Cluster Incl AW124843 UI-M-BH2.1.apk-f-10-0-UI s1 Mus musculus cDNA, 3 end/clone = UI-M-BH2 1-apk-f-10-0-UI/ | 204.9 | A | 460 | P | 469.9 | A |
| | | clone_end = 3/gb = AW124843/gi = 6100373/ug = Mm.28596/len = 509/STRA = for | | | | | | |
| 5 | 104639_i_at | Cluster Incl AI464596 mz66c12.y1 Mus musculus cDNA, 5 end/clone = IMAGE-718390/clone_end = 5/gb = AI464596/ | 197.9 | P | 8 | A | 94.1 | A |
| | | gi = 4318626/ug = Mm 31114/len = 339/STRA = for | | | | | | |
| 5 | 92724_at | Cluster Incl AI183202 uc50h11.r1 Mus musculus cDNA, 5 end/clone = IMAGE-1429125/clone_end = 5/gb = AI183202/ | 157.5 | P | 147.3 | P | 150.4 | P |
| | | gi = 3733840/ug = Mm.46512/len = 314/STRA = for | | | | | | |
| 5 | 93858_at | Cluster Incl M33266 Murine macrophage interferon inducible protein 10 (IP-10) mRNA, complete cds/cds = (40,336)/ | 62.3 | A | 46.5 | A | 65.6 | P |
| | | gb = M33266/gi = 198444/ug = Mm.877/len = 1063/STRA = rev | | | | | | |
| 5 | 96090_g_at | Cluster Incl AI255972 ui76a02 x1 Mus musculus cDNA, 3 end/clone = IMAGE-1888298/clone_end = 3/gb = AI255972/ | 3.1 | A | 26.8 | A | 37.2 | P |
| | | gi = 3863497/ug = Mm.26726/len = 511/STRA = for | | | | | | |
| 5 | 101998_at | Cluster Incl AW125086 UI-M-BH2 1-apx-f-03-0-UI s1 Mus musculus cDNA, 3 end/clone = UI-M-BH2 1-apx-f-03-0-UI/ | 75.3 | M | 88.1 | P | 75 | P |
| | | clone_end = 3/gb = AW125086/gi = 6100616/ug = Mm 9860/len = 461/STRA = for | | | | | | |
| 5 | 102030_at | Cluster Incl AF026032 X-linked nuclear protein/cds = (220,7650)/gb = AF026032/gi = 3002557/ug = Mm 10141/len = 7673/ | 34.9 | A | 63.5 | P | 33 | A |
| | | STRA = for | | | | | | |
| 5 | 102761_at | Cluster Incl AF041060 Mus musculus co-chaperone mt-GrpE#2 mRNA, nuclear gene encoding mitochondrial protein, | 174.4 | P | 129.8 | A | 168.8 | A |
| | | partial cds/cds = (0,671)/gb = AF041060/gi = 3411071/ug = Mm 12959/len = 1445/STRA = for | | | | | | |
| 5 | 103797_at | Cluster Incl AB019388: Cell divison cycle 7-like 1/cds-(86,1780)/gb-AB019388/gi = 3869101/ug = Mm 20842/len = 2708/ | 50.6 | P | 49.9 | A | 36.2 | P |
| | | STRA = rev | | | | | | |
| 5 | 98514_at | Cluster Incl AF004833 Mus musculus tissue factor pathway inhibitor (TFPI) mRNA, complete cds/cds = (104,1024)/ | 90.1 | P | 113.8 | A | 63.6 | A |
| | | gb = AF004833/gi = 2897076/ug = Mm 3601/len = 1288/STRA = for | | | | | | |
| 5 | 99465_at | Cluster Incl AJ132922: Mus musculus mRNA for protein encoded by methyl-CpG-binding protein 2/cds = (35,1489)/ | 128.1 | A | 110.9 | P | 182 | P |
| | | gb = AJ132922/gi = 5419961/ug = Mm.40984/len = 10087/STRA = for | | | | | | |
| 5 | 99507_at | Cluster Incl M21247 Uncoupling protein, mitochondrial/cds = (0,923)/gb = M21247/gi = 202268/ug = Mm 4177/len = 924/ | 71 | A | 281.2 | P | 451.5 | P |
| | | STRA = for | | | | | | |
| 5 | 100431_at | Cluster Incl U42467.Leptin receptor/cds = (60,2744)/gb = U42467/gi = 1139592/ug = Mm 4756/len = 4560/STRA = for | 29.1 | A | 22.3 | A | 31.1 | P |
| 5 | 100876_at | Cluster Incl AI841076 UI-M-AH0-acz-a-07-0-UI s1 Mus musculus cDNA, 3 end/clone = UI-M-AH0-acz-a-07-0-UI/ | 3.4 | P | −17 | A | −27.2 | A |
| | | clone_end = 3/gb = AI841076/gi = 5475289/ug = Mm 5264/len = 424/STRA = for | | | | | | |
| 5 | 103444_at | Cluster Incl AI272489.uk06e09 y1 Mus musculus cDNA, 5 end/clone = IMAGE-1958632/clone_end = 5/gb = AI272489/ | 68.1 | P | 63.1 | A | 60.1 | A |
| | | gi = 3894757/ug = Mm 21492/len = 459/STRA = for | | | | | | |
| 5 | 103763_at | Cluster Incl AW121855 UI-M-BH2 3-aoi-b-08-0-UI s1 Mus musculus cDNA, 3 end/clone = UI-M-BH2.3-aoi-b-08-0-UI/ | 72.5 | A | 198.5 | P | 192.2 | P |
| | | clone_end = 3/gb = AW121855/gi = 6097229/ug = Mm.42637/len = 462/STRA = for | | | | | | |
| 5 | 104478_at | Cluster Incl U27177 Retinoblastoma-like 1 (p107)/cds = (63,3254)/gb = U27177/gi = 1871224/ug = Mm 2994/len = 4863/ | 38 | A | 100.1 | A | 81.6 | A |
| | | STRA = for | | | | | | |
| 6 | 92585_at | Cluster Incl L02526 Protein kinase, mitogen activated, kinase 1, p45/cds = (40,1221)/gb = L02526/gi = 199123/ug = Mm.1059/ | 187.1 | P | 202 | P | 218.3 | P |
| | | len = 2119/STRA = for | | | | | | |
| 6 | 92586_at | Cluster Incl X57024: Glutamate dehydrogenase/cds = (76,1752)/gb = X57024/gi = 51081/ug = Mm 10600/len = 2942/ | 301.4 | P | 621.5 | P | 539.6 | P |
| | | STRA = for | | | | | | |
| 6 | 92597_s_at | Cluster Incl U13838 Mus musculus vacuolar adenosine triphosphatase subunit B gene, complete cds/cds = (0,1535)/ | 362.1 | P | 294.2 | P | 362.8 | P |
| | | gb = U13838/gi = 1184660/ug = Mm.10727/len = 1536/STRA = for | | | | | | |
| 6 | 92828_at | Cluster Incl AB004789 Mus musculus mRNA for dolichol-phosphate-mannose synthase, complete cds/cds = (0,782)/ | 381.1 | P | 472.6 | P | 403 | P |
| | | gb = AB004789/gi = 3073772/ug = Mm.12969/len = 789/STRA = for | | | | | | |
| 6 | 92847_s_at | Cluster Incl X56831: Mannose-6-phosphate receptor, cation dependent/cds = (98,934)/gb = X56831/gi = 53205/ug = Mm 1358/ | 435.6 | P | 581 | P | 562.3 | P |
| | | len = 1395/STRA = for | | | | | | |
| 6 | 92854_at | Cluster Incl D50500 Mouse mRNA for Rab 11, partial sequence/cds = (0,291)/gb = D50500/gi = 1742912/ug = Mm.1387/ | 1579.5 | P | 2235.6 | P | 1949.6 | P |
| | | len = 464/STRA = for | | | | | | |
| 6 | 93037_i_at | Cluster Incl M69260 Lipocortin 1/cds = (14,1054)/gb = M69260/gi = 198844/ug = Mm 14860/len = 1340/STRA = for | 992.8 | P | 1286.8 | P | 858.5 | P |
| 6 | 93043_at | Cluster Incl D50463 Mouse SDR1 mRNA, complete cds/cds = (27,872)/gb = D50463/gi = 1747303/ug = Mm 15125/len = 1900/ | 508.1 | P | 729.2 | P | 662 | P |
| | | STRA = for | | | | | | |
| 6 | 93066_at | Cluster Incl D16195 Granulin/cds = (0,1769)/gb = D16195/gi = 286067/ug = = Mm 1568/len = 1770/STRA = for | 322.9 | P | 350.9 | P | 346.7 | P |
| 6 | 93261_at | Cluster Incl AJ000990 Mus musculus mRNA for legumain/cds = (94,1401)/gb = AJ000990/gi = 3676226/ug = Mm 17185/ | 275.7 | P | 361.4 | P | 403.6 | P |
| | | len = 1889/STRA = for | | | | | | |
| 6 | 93290_at | Cluster Incl U35374 Mus musculus purine nucleoside phosphorylase (Np-b) mRNA, complete cds/cds = (0,869)/gb = U35374/ | 401.6 | P | 401.2 | P | 417.6 | P |
| | | gi = 114566/ug = Mm 17932/len = 870/STRA = for | | | | | | |
| 6 | 93309_at | Cluster Incl U42386: Fibroblast growth factor Inducible 14/cds = (880,1065)/gb = U42386/gi = 1353710/ug = Mm.18459/ | 344.2 | P | 482.7 | P | 337.5 | P |
| | | len = 2610/STRA = for | | | | | | |
| 6 | 93320_at | Cluster Incl AF017175: Camitine palmitoyltransferase 1, liver/cds = (0,2296)/gb = AF017175/gi = 2388721/ug = Mm.18522/ | 384.9 | P | 579.3 | P | 824.7 | P |
| | | len = 4414/STRA = for | | | | | | |
| 6 | 93347_at | Cluster Incl Z22819: RAB24, member RAS oncogene family/cds = (0,611)/gb = Z22819/gi = 438163/ug = Mm 18800/len = 660/ | 371 | P | 437.4 | P | 449.8 | P |
| | | STRA = for | | | | | | |
| 6 | 93364_at | Cluster Incl X59990: Calenin alpha 1/cds = (72,2792)/gb = X59990/gi = 49934/ug = Mm 18962/len = 3713/STRA = for | 657.4 | P | 930.4 | P | 715.7 | P |
| 6 | 93497_at | Cluster Incl K02782: Mouse complement component C3 mRNA, alpha and beta subunits, complete cds/cds = (56,5047)/ | 157.6 | P | 391.5 | P | 626.4 | P |
| | | gb = K02782/gi = 192278/ug = Mm 19131/len = 5067/STRA = for | | | | | | |
| 6 | 93498_s_at | Cluster Incl M97216: Mouse DNA-binding protein mRNA, complete cds/cds = (0,1535)/gb = M97216/gi = 515628/ | 1318.7 | P | 1730.6 | P | 1219.6 | P |
| | | ug = Mm 19133/len = 1536/STRA = for | | | | | | |
| 6 | 93499_at | Cluster Incl U16740: Mus musculus capping protein alpha 1 subunit mRNA, partial cds/cds = (0,856)/gb = U16740/ | 823.6 | P | 1100.3 | P | 980.3 | P |
| | | gi = 595916/ug = Mm 19142/len = 2893/STRA = for | | | | | | |
| 6 | 93818_g_at | Cluster Incl AB025218: Mus musculus mRNA for Sid394p, complete cds/cds = (33,638)/gb = AB025218/gi = 5931556/ | 1122.1 | P | 1818.9 | P | 1819.3 | P |
| | | ug = Mm 21507/len = 677/STRA = for | | | | | | |
| 6 | 93819_at | Cluster Incl AI847269: UI-M-AI1-afq-h-06-0-UI.s1 Mus musculus cDNA, 3 end/clone = UI-M-AI1-afq-h-06-0-UI/clone_end = 3/ | 1717 | P | 2152.9 | P | 2006.9 | P |
| | | gb = AI847269/gi = 5491175/ug = Mm.21507/len = 287/STRA = for | | | | | | |
| 6 | 93994_at | Cluster Incl AW212131: uo82g04.x1 Mus musculus cDNA, 3 end/clone = IMAGE-2649078/clone_end = 3/gb = AW212131/ | 77.9 | P | 206.1 | P | 220.3 | P |
| | | gi = 6518137/ug = Mm 21754/len = 566/STRA = for | | | | | | |
| 6 | 94002_at | Cluster Incl AI849838: UI-M-AL1-aho-d-08-0-UI.s1 Mus musculus cDNA, 3 end/clone = UI-M-AL1-aho-d-08-0-UI/ | 434.9 | P | 520.4 | P | 468.4 | P |
| | | clone_end = 3/gb = AI849838/gi = 5493737/ug = Mm 21771/len = 417/STRA = for | | | | | | |
| 6 | 94003_at | Cluster Incl AI848510: UI-M-AP1-agf-d-06-0-UI.s2 Mus musculus cDNA, 3 end/clone = UI-M-AP1-agf-d-06-0-UI/ | 639.6 | P | 744.1 | P | 630 | P |
| | | clone_end = 3/gb = AI848510/gi = 5492416/ug = Mm 21773/len = 420/STRA = for | | | | | | |
| 6 | 94041_at | Cluster Incl AI840643: UI-M-AM0-adq-g-08-0-UI.s1 Mus musculus cDNA, 3 end/clone = UI-M-AM0-adq-g-08-0-UI/ | 685.2 | P | 849 | P | 769.4 | P |
| | | clone_end = 3/gb = AI840643/gi = 5474851/ug = Mm.21953/len = 517/STRA = for | | | | | | |
| 6 | 95412_at | Cluster Incl U49112: Mus musculus calcium-binding protein ALG-2 (ALG-257) mRNA, complete cds/cds = (56,631)/ | 601.2 | P | 602 | P | 587.9 | P |
| | | gb = U49112/gi = 1213519/ug = Mm 24254/len = 1060/STRA = for | | | | | | |
| 6 | 95428_at | Cluster Incl AA688761: vr07b11.r1 Mus musculus cDNA, 5 end/clone = IMAGE-1111101/clone_end = 5/gb = AA883761/ | 306.6 | P | 284.7 | P | 310.5 | P |
| | | gi = 2678190/ug = Mm 24456/len = 322/STRA = for | | | | | | |
| 6 | 95434_at | Cluster Incl AI851740: UI-M-BH0-aim-a-03-0-UI.s1 Mus musculus cDNA, 3 end/clone = UI-M-BH0-aim-a-03-0-UI/ | 1031.3 | P | 1367.8 | P | 1263.3 | P |
| | | clone_end = 3/gb = AI851740/gi = 5495641/ug = Mm 24498/len = 373/STRA = for | | | | | | |
| 6 | 95444_at | Cluster Incl AW122274: UI-M-BH2.2-aos-a-04-0-UI.s1 Mus musculus cDNA, 3 end/clone = UI-M-BH2.2-aos-a-04-0-UI/ | 846.4 | P | 943.2 | P | 986.4 | P |
| | | clone_end = 3/gb = AW122274/gi = 6097751/ug = Mm 24584/len = 261/STRA = for | | | | | | |
| 6 | 95489_at | Cluster Incl AW047953: UI-M-BH1-aln-f-07-0-UI.s1 Mus musculus cDNA, 3 end/clone = UI-M-BH1-aln-f-07-0-UI/ | 236 | P | 278.5 | P | 214.4 | P |
| | | clone_end = 3/gb = AW047953/gi = 5908482/ug = Mm 25044/len = 331/STRA = for | | | | | | |
| 6 | 95507_at | Cluster Incl AB025048: Mus musculus mRNA for Sid6061p, complete cds/cds = (69,1025)/gb = AB025048/gi = 5931550/ | 178.6 | P | 242.7 | P | 171.1 | P |
| | | ug = Mm 25198/len = 1912/STRA = for | | | | | | |
| 6 | 96003_at | Cluster Incl AW048332: UI-M-BH1-akk-c-05-0-UI.s1 Mus musculus cDNA, 3 end/clone = UI-M-BH1-akk-c-05-0-UI/ | 780.6 | P | 633.9 | P | 686.7 | P |
| | | clone_end = 3/gb = AW048332/gi = 5908861/ug = Mm 25339/len = 333/STRA = for | | | | | | |
| 6 | 96007_at | Cluster Incl AI835359: UI-M-AQ0-aah-g-05-0-UI s1 Mus musculus cDNA, 3 end/clone = UI-M-AQ0-aah-g-05-0-UI/ | 1289.6 | P | 1371.5 | P | 1258 | P |
| | | clone_end = 3/gb = AI835359/gi = 5469572/ug = Mm 25393/len = 305/STRA = for | | | | | | |
| 6 | 96120 at | Cluster Incl AW124750: UI-M-BH2.1-aqa-e-04-0-UI s1 Mus musculus cDNA, 3 end/clone = UI-M-BH2.1-aqa-e-04-0-UI/ | 665.6 | P | 706.9 | P | 667.8 | P |
| | | clone_end = 3/gb = AW124750/gi = 6100280/ug = Mm 2701/len = 184/STRA = for | | | | | | |
| 6 | 96607_at | Cluster Incl AW124902: UI-M-BH2 1-apu-d-12-0-UI s1 Mus musculus cDNA, 3 end/clone = UI-M-BH2.1-apu-d-12-0-UI/ | 572.5 | P | 758.5 | P | 555.9 | P |
| | | clone_end = 3/gb = AW124902/gi = 6100432/ug = Mm 27065/len = 281/STRA = for | | | | | | |
| 6 | 96613_at | Cluster Incl AI852095: UI-M-BH0-aja-h-02-0-UI.s1 Mus musculus cDNA, 3 end/clone = UI-M-BH0-aja-h-02-0-UI/ | 389.6 | P | 376.7 | P | 372.4 | P |
| | | clone_end = 3/gb = AI852095/gi = 5496001/ug = Mm 27107/len = 381/STRA = for | | | | | | |
| 6 | 96675_at | Cluster Incl AW124245: UI-M-BH2.1-aph-f-06-0-UI.s1 Mus musculus cDNA, 3 end/clone = UI-M-BH2.1-aph-f-06-0-UI/ | 519.9 | P | 738.3 | P | 700.5 | P |
| | | clone_end = 3/gb = AW124245/gi = 6099775/ug = Mm 27404/len = 375/STRA = for | | | | | | |
| 6 | 96701_at | Cluster Incl U35833: Mus musculus anthracycline-associated resistance (Arx) mRNA, complete cds/cds = (20,1936)/ | 1040.4 | P | 925.9 | P | 968.5 | P |
| | | gb = U35833/gi = 4096673/ug = Mm.27560/len = 2525/STRA = for | | | | | | |
| 6 | 97262_at | Cluster Incl AW050305: UI-M-BH1-ans-g-11-0-UI.s1 Mus musculus cDNA, 3 end/clone = UI-M-BH1-ans-g-11-0-UI/ | 859.2 | P | 747.6 | P | 840.7 | P |
| | | clone_end = 3/gb = AW050305/gi = 5910834/ug = Mm 27899/len = 342/STRA = for | | | | | | |
| 6 | 97829_at | Cluster Incl AI838053: UI-M-AL0-abv-c-03-0-UI s1 Mus musculus cDNA, 3 end/clone = UI-M-AL0-abv-c-03-0-UI/ | 281.6 | P | 286.8 | P | 215.6 | P |
| | | clone_end = 3/gb = AI836053/gi = 5472266/ug = Mm 28219/len = 367/STRA = for | | | | | | |
| 6 | 97844_at | Cluster Incl U67187: Mus musculus G protein signaling regulator RGS2 (rgs2) mRNA, complete cds/cds = (20,655)/ | 278.3 | P | 215 | P | 273.6 | P |
| | | gb = U67187/gi = 1911236/ug = Mm 28262/len = 1240/STRA = for | | | | | | |
| 6 | 97904_at | Cluster Incl AW 123953: UI-M-BH2.3-aqc-d-06-0-UI.s1 Mus musculus cDNA, 3 end/clone = UI-M-BH2.3-aqc-d-06-0-UI/ | 1718.9 | P | 1952.5 | P | 1896.9 | P |
| | | clone_end = 3/gb = AW123953/gi = 6099483/ug = Mm.28464/len = 382/STRA = for | | | | | | |
| 6 | 94452_g_at | Cluster Incl AI787627: ul20f06.y1 Mus musculus cDNA, 5 end/clone = IMAGE-2088131/clone_end = 5/gb = AI787627/ | 696.7 | P | 787.8 | P | 778.4 | P |
| | | gi = 5335343/ug = Mm 28786/len = 450/STRA = for | | | | | | |
| 6 | 94461_at | Cluster Incl AI852144: UI-M-BH0-ajb-e-09-0-UI.s1 Mus musculus cDNA, 3 end/clone = UI-M-BH0-ajb-e-09-0-UI/ | 179.4 | P | 168 | P | 158.8 | P |
| | | clone_end = 3/gb = AI652144/gi = 5496055/ug = Mm 28830/len = 425/STRA = for | | | | | | |
| 6 | 94502_at | Cluster Incl AW125724: UI-M-BH2 2-aql-d-12-0-UI.s1 Mus musculus cDNA, 3 end/clone = UI-M-BH2.2-aql-d-12-0-UI/ | 142.4 | P | 103.5 | P | 155.3 | P |
| | | clone_end = 3/gb = AW125724/gi = 8101294/ug = Mm 2895/len = 460/STRA = for | | | | | | |
| 6 | 95070_at | Cluster Incl AW125874: UI-M-BH2 2-aqk-f-09-0-UI s1 Mus musculus cDNA, 3 end/clone = UI-M-BH2.2-aqk-f-09-0-UI/ | 231.2 | P | 333.8 | P | 270.4 | P |
| | | clone_end = 3/gb = AW125874/gi = 6101404/ug = Mm 29192/len = 266/STRA = for | | | | | | |
| 6 | 95086_at | Cluster Incl AI835392: UI-M-AQ0-aaj-b-10-0-UI.s1 Mus musculus cDNA, 3 end/clone = UI-M-AQ0-aaj-b-10-0-UI/ | 781.1 | P | 896.8 | P | 948.3 | P |
| | | clone_end = 3/gb = AI835392/gi = 5469605/ug = Mm 29259/len = 221/STRA = for | | | | | | |
| 6 | 95140_at | Cluster Incl AI842068: UI-M-AN1-afg-b-01-0-UI.s1 Mus musculus cDNA, 3 end/clone = UI-M-AN1-afg-b-01-0-UI/ | 653.7 | P | 430 | P | 514.5 | P |
| | | clone_end = 3/gb = AI842068/gi = 5476281/ug = Mm 29436/len = 397/STRA = for | | | | | | |
| 6 | 95148_at | Cluster Incl AB020202: Mus musculus mRNA for adenylate kinase isozyme 2, complete cds/cds = (42,740)/gb = AB020202/ | 314.5 | P | 326.1 | P | 354.5 | P |
| | | gi = 4760597/ug = Mm.29460/len = 983/STRA = for | | | | | | |
| 6 | 95654_at | Cluster Incl AF109905: Mus musculus major histocompatibility locus class III regions Hsc70t gene, partial cds; smRNP, | 1128.9 | P | 1128.6 | P | 1048.7 | P |
| | | G7A, NG23, MutS homolog, CLCP, NG24, NG25, and NG26 genes, complete cds, and unknown genes/cds = (0,725)/ | | | | | | |
| | | gb = AF109905/gi = 3986751/ug = Mm 29/STRA = for | | | | | | |
| 6 | 95708_at | Cluster Incl AI843466 UI-M-AQ1-aec-g-01-0-UI s1 Mus musculus cDNA, 3 end/clone = UI-M-AQ1-aoc-g-01-0-UI/ | 606.8 | P | 1197.8 | P | 906.5 | P |
| | | clone_end = 3/gb = AI843466/gi = 5477684/ug = Mm 29702/len = 325/STRA = for | | | | | | |
| 6 | 96281_at | Cluster Incl AW121246 UI-M-BH2 3-aok-g-05-0-UI s1 Mus musculus cDNA, 3 end/clone = UI-M-BH2 3-aok-g-05-0-UI/ | 2014.3 | P | 1866.2 | P | 2291.4 | P |
| | | clone_end = 3/gb = AW121246/gi = 6096579/ug = Mm 29868/len = 355/STRA = for | | | | | | |
| 6 | 96333_g_at | Cluster Incl AW259199.um91a03 y1 Mus musculus cDNA, 5 end/clone = lMAGE-2332108/clone_end = 5/gb = AW259199/ | 268.1 | P | 454.6 | P | 281.8 | P |
| | | gi = 6632180/ug = Mm 29971/len = 411/STRA = for | | | | | | |
| 6 | 96348_at | Cluster Incl AW121217.UI-M-BH2 3-aok-d-10-0-UI s1 Mus musculus cDNA, 3 end/clone = UI-M-BH2 3-aok-d-10-0-UI/ | 694.3 | P | 780.3 | P | 1018.3 | P |
| | | clone_end = 3/gb = AW121217/gi = 6096550/ug = Mm 29998/len = 454/STRA = for | | | | | | |
| 6 | 96353_at | Cluster Incl AW125346 UI-M-BH2 1-apy-h-02-0-UI s1 Mus musculus cDNA, 3 end/clone = UI-M-BH2 1-apy-h-02-0-UI/ | 521.7 | P | 435.9 | P | 464.4 | P |
| | | clone_end = 3/gb = AW125346/gi = 6100876/ug = Mm 30005/len = 382/STRA = for | | | | | | |
| 6 | 96913_at | Cluster Incl AW122615 UI-M-BH2.2 aol-b-06-0-UI s1 Mus musculus cDNA, 3 end/clone = UI-M-BH2 2-aol-b-06-0-UI/ | 863.4 | P | 946.1 | P | 1027.8 | P |
| | | clone_end = 3/gb = AW1226l5/gi = 6098142/ug = Mm.30147/len = 244/STRA = for | | | | | | |
| 6 | 96936_at | Cluster Incl AI020792 ub04g10 r1 Mus musculus cDNA, 5 end/clone = IMAGE-1366050/clone_end = 5/gb = AI020792/ | 347.6 | P | 420 | P | 317.1 | P |
| | | gi = 3235128/ug = Mm 30183/len = 421/STRA = for | | | | | | |
| 6 | 97486_at | Cluster Incl AA693246 vr58e09.s1 Mus musculus cDNA, 5 end/clone = lMAGE-1124872/clone_end = 5/gb = AA693246/ | 721.3 | P | 844 | P | 792.8 | P |
| | | gi = 2694184/ug = Mm 30926/len = 397/STRA = for | | | | | | |
| 6 | 98101_at | Cluster Incl AI851798 UI-M-BH0-ais-a-06-0-UI.s1 Mus musculus cDNA, 3 end/clone = UI-M-BH0-als-a-06-0-UI/ | 203.5 | P | 318.5 | P | 287.7 | P |
| | | clone_end = 3/gb = AI851798/gi = 5495704/ug = Mm.34772/len = 300/STRA = for | | | | | | |
| 6 | 98102_at | Cluster Incl M76727: Pyruvate dehydrogenase E1 alpha subunit/cds = (111,1283)/gb = M76727/gi = 2002768/ug = Mm 34775/ | 1000.3 | P | 1315.8 | P | 1133.9 | P |
| | | len = 1446/STRA = for | | | | | | |
| 6 | 98521_at | Cluster Incl AI847972 UI-M-AI1-all-h-04-0-UI s1 Mus musculus cDNA, 3 end/clone = UI-M-AI1-all-h-04-0-UI/clone_end = 3/ | 558.5 | P | 546.4 | P | 542.3 | P |
| | | gb = AI847972/gi = 5491878/ug = Mm.35808/len = 172/STRA = for | | | | | | |
| 6 | 98525_f_at | Cluster Incl AJ007909.Mus musculus mRNA for erythroid differentiation regulator, partial/cds = (154,687)/gb = AJ007909/ | 1379.3 | P | 1442.9 | P | 1334 | P |
| | | gi = 3336885/ug = Mm.35829/len = 715/STRA = for | | | | | | |
| 6 | 99158_at | Cluster Incl U58888 Mus musculus SH3-containing protein SH3P2 mRNA, partial cds/cds = (0,755)/gb = U58888/ | 502.4 | P | 512.5 | P | 465.1 | P |
| | | gi = 1407662/ug = Mm 4165/len = 1041/STRA = for | | | | | | |
| 6 | 99169_at | Cluster Incl AW122165 UI-M-BH2.2-aor-g-07-0-UI s1 Mus musculus cDNA, 3 end/clone = UI-M-BH2.2-aor-g-07-0-UI/ | 435 | P | 590.2 | P | 439.1 | P |
| | | clone_end = 3/gb = AW122165/gi = 6097606/ug = Mm.41705/len = 204/STRA = for | | | | | | |
| 6 | 99587_at | Cluster Incl X89650 RAB7, member RAS oncogene family/cds = (156,779)/gb = X89650/gi = 1050550/ug = Mm.4268/ | 1011.2 | P | 1340.7 | P | 840.6 | P |
| | | len = 2089/STRA = for | | | | | | |
| 6 | 99610_at | Cluster Incl X93357 Synovial sarcoma, translocated to X Chromosome/cds = (179,1435)/gb = X93357/gi = 1072383/ | 461.5 | P | 735.4 | P | 632.4 | P |
| | | ug = Mm 4296/len = 3107/STRA = for | | | | | | |
| 6 | 100572_at | Cluster Incl AI851289 UI-M-BH0-akf-f-05-0-UI s1 Mus musculus cDNA, 3 end/clone = UI-M-BH0-akf-f-05-0-UI/ | 434.4 | P | 524.6 | P | 434.7 | P |
| | | clone_end = 3/gb = AI851289/gi = 5495195/ug = Mm.5885/len = 449/STRA = for | | | | | | |
| 6 | 100595_at | Cluster Incl AF035644: Mus musculus potentially prenylated protein tyrosine phosphatase mPRL-2 (Prl2) mRNA, complete | 1205.3 | P | 1502.6 | P | 1637.7 | P |
| | | cds/cds = (179,682)/gb = AF035644/gi = 2992629/ug = Mm 6355/len = 723/STRA = for | | | | | | |
| 6 | 100603_at | Cluster Incl AF063231: Dynein, cytoplasmic, intermediate chain 2/cds = (77,1915)/gb = AF063231/gi = 3493590/ | 93.1 | P | 146.1 | P | 100.4 | P |
| | | ug = Mm 6441/len = 2458/STRA = for | | | | | | |
| 6 | 101040_at | Cluster Incl D38117: Mus musculus mRNA for m-calpain large subunit, complete cds/cds = (170,2272)/gb = D38117/ | 538.7 | P | 793.9 | P | 788.4 | P |
| | | gi = 2570157/ug = Mm 6958/len = 3225/STRA = for | | | | | | |
| 6 | 101982_at | Cluster Incl X98475 M musculus VASP gene/cds = (0,1130)/gb = X98475/gi = 1617401/ug = Mm.9684/len = 1131/STRA = for | 613.3 | P | 769.8 | P | 787.3 | P |
| 6 | 102407_at | Cluster Incl M68898: Mast cell protease 5/cds = (5,748)/gb = M68898/gi = 199028/ug = Mm 1252/len = 960/STRA = for | 163.4 | P | 77.2 | P | 35.2 | P |
| 6 | 102790_at | Cluster Incl AB016490 Mus musculus mJTB mRNA, complete cds/cds = (306,746)/gb = AB016490/gi = 3721891/ | 1039.3 | P | 1254.7 | P | 1093.8 | P |
| | | ug = Mm 13912/len = 1273/STRA = for | | | | | | |
| 6 | 103018_at | Cluster Incl AF096868 Mus musculus transcytosis associated protein p115 mRNA, partial cds/cds = (0,2827)/gb = AF096868/ | 148 | P | 182.2 | P | 185.8 | P |
| | | gi = 3860050/ug = Mm 15868/len = 3690/STRA = for | | | | | | |
| 6 | 103899_at | Cluster Incl AA690863 vt32b05 r1 Mus musculus cDNA, 5 end/clone = IMAGE-1164753/clone_end = 5/gb = AA690863/ | 172.7 | P | 323.6 | P | 257.5 | P |
| | | gi = 2691799/ug = Mm 21313/len = 289/STRA = for | | | | | | |
| 6 | 104315_at | Cluster Incl AI846773 UI-M-AO1-ael-f-02-0-UI s1 Mus musculus cDNA, 3 end/clone = UI-M-AO1-ael-f-02-0-UI/ | 245 | P | 333.4 | P | 266.6 | P |
| | | clone_end = 3/gb = AI846773/gi = 5490679/ug = Mm.22413/len = 322/STRA = for | | | | | | |
| 6 | 104350_at | Cluster Incl AI050321 ub29c11 r1 Mus musculus cDNA, 5 end/clone = IMAGE-1379158/clone_end = 5/gb = AI050321/ | 338 | P | 321.6 | P | 331.2 | P |
| | | gi = 3299438/ug = Mm.22550/len = 504/STRA = for | | | | | | |
| 6 | 104612_g_at | Cluster Incl AI854008 UI-M-BH0-aiv-h-12-0-UI s1 Mus musculus cDNA, 3 end/clone = UI-M-BH0-aiv-h-12-0-UI/ | 166.8 | P | 261.8 | P | 244.5 | P |
| | | clone_end = 3/gb = AI854008/gi = 5497914/ug = Mm.24158/len = 494/STRA = for | | | | | | |
| 6 | 94369_at | Cluster Incl AW123026 UI-M-BH2 1-apb-f-07-0-UI s1 Mus musculus cDNA, 3 end/clone = UI-M-BH2 1-apb-f-07-0-UI/ | 133.8 | P | 135.6 | P | 145.1 | P |
| | | clone_end = 3/gb = AW123026/gi = 6098556/ug = Mm.25336/len = 442/STRA = for | | | | | | |
| 6 | 94408_at | Cluster Incl U47008: Ngfi-A binding protein 1/cds = (136,1596)/gb = U47008/gi = 1197670/ug = Mm 25903/len = 1796/ | 1125.9 | P | 1136 | P | 971.4 | P |
| | | STRA = for | | | | | | |
| 6 | 94427_at | Cluster Incl AI841737 UI-M-AL0-abo-g-05-0-UI.s1 Mus musculus cDNA, 3 end/clone = UI-M-AL0-abo-g-05-0-UI/ | 186.7 | P | 211.4 | P | 228.5 | P |
| | | clone_end = 3/gb = AI841737/gi = 5475950/ug = Mm.26422/len = 372/STRA = for | | | | | | |
| 6 | 94963_at | Cluster Incl AI462105 ub70b01 x1 Mus musculus cDNA, 3 end/clone = IMAGE-1383049/clone_end = 3/gb = AI462105/ | 987.4 | P | 1509.1 | P | 1011.9 | P |
| | | gi = 4316135/ug = Mm.27206/len = 422/STRA = for | | | | | | |
| 6 | 95608_at | Cluster Incl AI851255 UI-M-BH0-akf-c-03-0-UI.s1 Mus musculus cDNA, 3 end/clone = UI-M-BH0-akf-c-03-0-UI/ | 5.4 | P | 194.1 | P | 131 | P |
| | | clone_end = 3/gb = AI851255/gi = 5495161/ug = Mm.27986/len = 427/STRA = rev | | | | | | |
| 6 | 96239_at | Cluster Incl AA815503 vp20d05.r1 Mus musculus cDNA, 5 end/clone = IMAGE-1069161/clone_end = 5/gb = AA815503/ | 162.4 | P | 99.6 | P | 182.4 | P |
| | | gi = 2885099/ug = Mm.28816/len = 477/STRA = for | | | | | | |
| 6 | 97939_at | Cluster Incl AW125079: UI-M-BH2.1-apx-e-08-0-UI.s1 Mus musculus cDNA, 3 end/clone = UI-M-BH2.1-apx-e-08-0-UI/ | 596.4 | P | 723.6 | P | 721.2 | P |
| | | clone_end = 3/gb = AW125079/gi = 6100609/ug = Mm.30265/len = 316/STRA = for | | | | | | |
| 6 | 97949_at | Cluster Incl M16238: Fibrinogen-like protein 2/cds = (60,1358)/gb = M16238/gi = 193305/ug = Mm 3040/len = 3772/STRA = for | 171.3 | P | 251.3 | P | 222.5 | P |
| 6 | 98466_r_at | Cluster Incl M31419 Interferon activated gene 204/cds = (127,2049)/gb = M31419/gi = 198399/ug = Mm 34817/len = 2353/ | 133.4 | P | 260.8 | P | 235.6 | P |
| | | STRA = for | | | | | | |
| 6 | 99051_at | Cluster Incl M36579 S100 calcium-binding protein A4/cds = (0,164)/gb = M36579/gi = 199816/ug = Mm 3925/len = 302/ | 259.3 | P | 277.6 | P | 581.2 | P |
| | | STRA = for | | | | | | |
| 6 | 99503_at | Cluster Incl AW045204 UI-M-BH1-alh-c-10-0-UI s1 Mus musculus cDNA, 3 end/clone = UI-M-BH1-alh-c-10-0-UI/ | 92.5 | P | 190 | P | 246.6 | P |
| | | clone_end = 3/gb = AW045204/gi = 5905733/ug = Mm.41714/len = 405/STRA = for | | | | | | |
| 6 | 99996_at | Cluster Incl Y07941: M musculus mRNA for plakophilin 1/cds = (12,2198)/gb = Y07941/gi = 1707593/ug = Mm 4494/len = 4504/ | 1567.9 | P | 1698.6 | P | 1575.3 | P |
| | | STRA = rev | | | | | | |
| 6 | 101867_at | Cluster Incl U11680 Mus musculus glycerol-3-phosphate acyltransferase gene, nuclear gene encoding mitochondrial/ | 542.7 | P | 596.4 | P | 547.5 | P |
| | | protein, 5 region and partial cds/cds = (411,425)/gb = U11680/gi = 786006/ug = Mm 87773/len = 426/STRA = rev | | | | | | |
| 6 | 101886_f_at | Cluster Incl X52490 Synthetic D(b) mRNA for D(b) glycoprotein (alpha chain) and beta(2) microglobulin/cds = (9,1097)/ | 997.3 | P | 1254.4 | P | 1293.7 | P |
| | | gb = X52490/gi = 50671/ug = Mm.88493/len = 1102/STRA = rev | | | | | | |
| 6 | 101948_at | Cluster Incl X05212 Mouse mRNA for laminin B1 C-terminus/cds = (0,1489)/gb = X05212/gi = 52861/ug = Mm.963/len = 1640/ | 463.5 | P | 476.4 | P | 353 | P |
| | | STRA = for | | | | | | |
| 6 | 102658_at | Cluster Incl X59769 Interleukin 1 receptor, type II/cds = (92,1324)/gb = X59769/gi = 52670/ug = Mm 1349/len = 1341/ | 211.8 | P | 146 | P | 133.6 | P |
| | | STRA = for | | | | | | |
| 6 | 103471_at | Cluster Incl AI194333 ui15d12 r1 Mus musculus cDNA, 5 end/clone = IMAGE-1851383/clone_end = 5/gb = AI194333/ | 142.4 | P | 226.1 | P | 176.4 | P |
| | | gi = 3745540/ug = Mm 21821/len = 459/STRA = for | | | | | | |
| 6 | 103744_at | Cluster Incl AI852760 UI-M-BH0-ary-b-12-0-UI s1 Mus musculus cDNA,3 end/clone = UI-M-BH0-ary-b-12-0-UI/ | 57.3 | P | 50.8 | P | 81 | P |
| | | clone_end = 3/gb = AI852760/gi = 5496666/ug = Mm 24472/len = 417/STRA = for | | | | | | |
| 6 | 104249_g_at | Cluster Incl AW227650 up 12b09 y1 Mus musculus cDNA, 5 end/clone = IMAGE-2654105/clone_end = 5/gb = AW227650/ | 342.4 | P | 457.5 | P | 372 | P |
| | | gi = 6556946/ug = Mm.28345/len = 440/STRA = for | | | | | | |
| 6 | 93721_at | Cluster Incl L12367 Mus musculus adenylyl cyclase-associated protein (CAP) mRNA, complete cds/cds = (64,1488)/ | 313 | P | 321 | P | 303.5 | P |
| | | gb = L12367/gi = 293316/ug = Mm 8687/len = 1563/STRA = for | | | | | | |
| 6 | 93871_at | Cluster Incl L32838 Mouse germline interleukin 1 receptor antagonist (IL-1m) gene,complete cds/cds = (0,536)/gb = L32838/ | 199.3 | P | 248.6 | P | 152.9 | P |
| | | gi = 487864/ug = Mm.882/len = 537/STRA = for | | | | | | |
| 6 | 99325_at | Cluster Incl M32502. Wingless-related MMTV integration site 3/cds = (45,1112)/gb = M32502/gi = 198428/ug= Mm 5188/ | 224.3 | P | 200 | P | 236.3 | P |
| | | len = 3000/STRA = for | | | | | | |
| 6 | 101741_at | Cluster Incl AB003305: Proteasome beta type subunit 5, pseudogene/cds = (0,797)/gb = AB003305/gi = 2897807/ | 486.8 | P | 983.9 | P | 461.8 | P |
| | | ug = Mm 57067/len = 798/STRA = for | | | | | | |
| 6 | 97593_f_at | Cluster Incl C79694 C79694 Mus musculus cDNA, 3 end/clone = J0070F03/clone_end = 3/gb = C79694/gi = 2520024/ | 347.8 | P | 447.8 | P | 484.2 | P |
| | | ug = Mm 89577/len = 439/STRA = rev | | | | | | |
| 6 | 101697_f_at | Cluster Incl AI844607 UI-M-AJ1-ahh-d-12-0-UI s2 Mus musculus cDNA, 3 end/clone = UI-M-AJ1-ahh-d-12-0-UI/ | 227.1 | P | 284.5 | P | 298.8 | P |
| | | clone_end = 3/gb = AI844607/gi = 5488513/ug = Mm.39586/len = 121/STRA = rev | | | | | | |
| 6 | 94084_at | Cluster Incl AI842888 UI-M-AN1-afb-f-12-0-UI s1 Mus musculus cDNA, 3 end/clone = UI-M-AN1-afb-f-12-0-UI/ | 165 | P | 154.8 | P | 127.5 | P |
| | | clone_end = 3/gb = AI842888/gi = 5477101/ug = Mm 22192/len = 331/STRA = for | | | | | | |
| 6 | 96103_f_at | Cluster Incl X92411: RAD23b homolog (S cerevisiae)/cds = (0,1250)/gb = X92411/gi = 1044898/ug = Mm 26879/len = 1251/ | 113.3 | P | −37.7 | A | 334.4 | P |
| | | STRA = for | | | | | | |
| 6 | 97480_f_at | Cluster Incl U95607 Mus musculus testis specific DNA-homolog mRNA, complete cds/cds = (72,800)/gb = U95607/ | 35.9 | P | 55.3 | P | 28 | P |
| | | gi = 2286122/ug = Mm 3075/len = 1030/STRA = for | | | | | | |
| 6 | 98099_at | Cluster Incl AW045760 UI-M-BH1-akt-b-12-0-UI.s1 Mus musculus cDNA, 3 end/clone = UI-M-BH1-akt-b-12-0-UI/ | 142.8 | P | 143.9 | P | 146.5 | P |
| | | clone_end = 3/gb = AW045760/gi = 5906289/ug = Mm. 34750/len = 413/STRA = for | | | | | | |
| 6 | 100059_at | Cluster Incl M31775 Mouse cytochrome beta-558 mRNA, 3 end/cds = (0,574)/gb = M31775/gi = 192913/ug = Mm 448/ | 706.2 | P | 445.3 | A | 564.4 | P |
| | | len = 677/STRA = for | | | | | | |
| 6 | 101106_at | Cluster Incl AI853331.UI-M-BH0-air-a-07-0-UI s1 Mus musculus cDNA, 3 end/clone = UI-M-BH0-air-a-07-0-UI/ | 117.8 | A | 180.2 | P | 208.7 | P |
| | | clone_end = 3/gb = AI853331/gi = 5497237/ug = Mm.7514/len = 347/STRA = for | | | | | | |
| 6 | 102012_at | Cluster Incl AB014485 Mus musculus mRNA for RA70, complete cds/cds = (65,1141)/gb = AB014485/gi = 4730810/ | 297 | P | 368.3 | P | 424.8 | P |
| | | ug = Mm 10019/len = 1554/STRA = for | | | | | | |
| 6 | 102813_f_at | Cluster Incl Aw210346 ul53g12 y1 Mus musculus cDNA, 5 end/clone = IMAGE-2102086/clone_end = 5/gb = AW210346/ | 152.6 | P | 208.2 | P | 114.7 | A |
| | | gi = 6516286/ug = Mm 14253/len = 431/STRA = for | | | | | | |
| 6 | 103347_at | Cluster Incl AI852548 UI-M-BH0-arw-f-09-0-UI s1 Mus musculus cDNA, 3 end/clone = UI-M-BH0-alw-f-09-0-UI/ | 47.8 | P | 150.8 | A | 231.4 | P |
| | | clone_end = 3/gb = AI852548/gi = 5496454/ug = Mm.1828/len = 350/STRA = for | | | | | | |
| 6 | 103905_at | Cluster Incl AI314958 uj34107.x1 Mus musculus cDNA, 3 end/clone = IMAGE-1921861/clone_end = 3/gb = AI314958/ | 33.9 | P | 146.8 | P | 184.4 | P |
| | | gi = 4030225/ug = Mm.21397/len = 487/STRA = rev | | | | | | |
| 6 | 104154_at | Cluster Incl AB021961 Mus musculus mutant p53 mRNA, complete cds/cds = (100,1272)/gb = AB021961/gi = 5421849/ | 312.3 | P | 365.8 | P | 447.3 | P |
| | | ug = Mm 222/len = 1429/STRA = for | | | | | | |
| 6 | 104761_at | Cluster Incl AA612450: vo70h07 r1 Mus musculus cDNA, 5 end/clone = IMAGE-1064509/clone_end = 5/gb = AA612450/ | 163.1 | P | 114.3 | P | 274.6 | P |
| | | gi = 2461128/ug = Mm.24842/len = 294/STRA = for | | | | | | |
| 6 | 94390_at | Cluster Incl AI646981 ub68c08 x1 Mus musculus cDNA, 3 end/clone = IMAGE-1382894/clone_end = 3/gb = AI646981/ | 130.8 | P | 240.7 | P | 189.4 | P |
| | | gi = 4725456/ug = Mm.25645/len = 540/STRA = for | | | | | | |
| 6 | 94931_at | Cluster Incl AI851104 UI-M-BH0-akg-c-11-0-UI s1 Mus musculus cDNA, 3 end/clone = UI-M-BH0-akg-c-11-0-UI/ | 180 | P | 227.1 | A | 210.5 | P |
| | | clone_end = 3/gb = AI851104/gi = 5495010/ug = Mm 26697/len = 378/STRA = for | | | | | | |
| 6 | 95592_at | Cluster Incl AW046785 UI-M-BH1-alz-a-05-0-UI s1 Mus musculus cDNA, 3 end/clone = UI-M-BH1-alz-a-05-0-UI/ | 214.5 | P | 234.5 | P | 284.8 | P |
| | | clone_end = 3/gb = AW046785/gi = 5907314/ug = Mm 279/len = 454/STRA = for | | | | | | |
| 6 | 96812_at | Cluster Incl AF089721: Smoothened homolog (Drosophila)/cds = UNKNOWN/gb = AF089721/gi = 3608491/ug = Mm 29279/ | −24.6 | P | 61.6 | P | 15.8 | P |
| | | len = 1073/STRA = for | | | | | | |
| 6 | 99019_at | Cluster Incl D17571. P450 (cytochrome) oxidoreductase/cds = (52,2088)/gb = D17571/gi = 407342/ug = Mm 3863/len = 2457/ | 178.4 | P | 348 | P | 323.6 | P |
| | | STRA = rev | | | | | | |
| 6 | 103441 _at | Cluster Incl AI194248: ui14a03.r1 Mus musculus cDNA, 5 end/clone = IMAGE-1851244/clone_end = 5/gb = AI194248/ | 150.6 | P | 151.1 | P | 125 | P |
| | | gi = 3745455/ug = Mm.21431/len = 438/STRA = for | | | | | | |
| 6 | 92481_at | Cluster Incl AF086905: Mus musculus protein kinase Chk2 (Chk2) mRNA, complete cds/cds = (0,1640)/gb = AF086905/ | 91.9 | P | 158.1 | P | 134.1 | P |
| | | gi = 3982841/ug = Mm 42149/len = 1821/STRA = for | | | | | | |
| 6 | 97197_r_at | Cluster Incl C78850 C78850 Mus musculus cDNA, 3 end/clone = J0056C12/clone_end = 3/gb = C78850/gi = 2519180/ | 367.4 | P | 439.1 | P | 456.2 | P |
| | | ug = Mm 36896/len = 219/STRA = rev | | | | | | |
| 6 | 99378_f_at | Cluster Incl M18837: Mouse MHC class I Q4 beta-2-microglobulin (Qb-1) gene, complete cds/cds = (0,980)/gb = M18837/ | 345.3 | P | 421.2 | P | 485.5 | P |
| | | gi = 199323/ug = Mm 56926/len = 981/STRA = for | | | | | | |
| 6 | 101816_at | Cluster Incl AF063937 Squamous cell carcinoma antigen 2/cds = (72,1238)/gb = AF063937/gi = 3885487/ug = Mm 57172/ | 455 | P | 471.4 | P | 430.4 | P |
| | | len = 1239/STRA = for | | | | | | |
| 6 | 102161_f_at | Cluster Incl X58609 Mouse MHC (Qa) Q2-k gene for class I antigen, exons 1-3/cds = (0,1088)/gb = X58609/gi = 53854/ | 329.2 | P | 382.2 | P | 495.1 | P |
| | | ug = Mm 46812/len = 1089/STRA = for | | | | | | |
| 6 | 93017_at | Cluster Incl AF077527 Mus musculus syntenin mRNA, complete cds/cds = (54,950)/gb = AF077527/gi = 3342559/ | 62.8 | P | 81.5 | P | 39.7 | P |
| | | ug = Mm 14744/len = 2043/STRA = for | | | | | | |
| 6 | 93590_at | Cluster Incl AI844370 UI-M-AL1-ahp-b-12-0-UI s1 Mus musculus cDNA, 3 end/clone = UI-M-AL1-ahp-b-12-0-UI/ | 143.3 | A | 164.3 | P | 238.1 | P |
| | | clone_end = 3/gb = AI844370/gi = 5488276/ug = Mm 20818/len = 430/STRA = for | | | | | | |
| 6 | 93775_at | Cluster Incl AI841894 UI-M-AO0-acd-d-09-0-UI s1 Mus musculus cDNA, 3 end/clone = UI-M-AO0-acd-d-09-0-UI/ | 6.7 | P | 62.1 | A | 55.9 | P |
| | | clone_end = 3/gb = AI841894/gi = 5476107/ug = Mm.2121/len = 435/STRA = for | | | | | | |
| 6 | 94298_at | Cluster Incl X53619 M musculus P35B gene, exon 1/cds = (0,815)/gb = X53619/gi = 53556/ug = Mm 22596/len = 816/ | 183.4 | P | 175.9 | A | 262 | P |
| | | STRA = for | | | | | | |
| 6 | 97876_at | Cluster Incl AI842008 UI-M-AH0-ada-c-09-0-UI.s1 Mus musculus cDNA, 3 end/clone = UI-M-AH0-ada-c-09-0-UI/ | 338.7 | A | 344.3 | P | 404.3 | P |
| | | clone_end = 3/gb = AI842008/gi = 5476221/ug = Mm.28356/len = 431/STRA = for | | | | | | |
| 6 | 97515_at | Cluster Incl X89998 M musculus mRNA for 17 beta-hydroxysteroid dehydrogenase type IV/cds = (12,2219)/gb = X89998/ | 241.7 | P | 244.9 | A | 210.8 | P |
| | | gi = 1213007/ug = Mm 3195/len = 2468/STRA = for | | | | | | |
| 6 | 97541_f_at | Cluster Incl X00246 Mouse mRNA with a Set 1 repetitive element for a class I major histocompatibility complex(MHC) | −56.2 | A | 9.4 | P | −17.9 | P |
| | | antigen/cds = (0,857)/gb = X00246/gi = 53085/ug = Mm.33263/len = 1484/STRA = rev | | | | | | |
| 6 | 98088_at | Cluster Incl X13333 CD14 antigen/cds = (32,1132)/gb = X13333/gi = 50338/ug = Mm.3460/len = 1366/STRA = for | 677.3 | M | 594.3 | P | 633.1 | P |
| 6 | 98092_at | Cluster Incl AA790307 vw17a10 r1 Mus musculus cDNA, 5 end/clone = IMAGE-1244058/clone_end = 5/gb = AA790307/ | −57.3 | A | 167.1 | P | 947.1 | P |
| | | gi = 2850427/ug = Mm.34609/len = 581/STRA = for | | | | | | |
| 6 | 98589_at | Cluster Incl M93275 Adipose differentiation related protein/cds = (78,1335)/gb = M93275/gi = 191692/ug = Mm 381/len = 1680/ | 691.7 | A | 1125 | P | 1111.6 | P |
| | | STRA = for | | | | | | |
| 6 | 101540_at | Cluster Incl AF069519: Mus musculus T-G mismatch-specific thymine-DNA glycosylase TDGb isoform mRNA, complete | 272.9 | A | 379.1 | P | 519 | P |
| | | cds/cds = (108,1301)/gb = AF069519/gi = 3420292/ug = Mm 88099/len = 2859/STRA = for | | | | | | |
| 6 | 101566_f_at | Cluster Incl M17818 Mouse major urinary prolein group 1 gene, 5 end, clone BL6-3/cds = (66,95)/gb = M17818/gi = 202305/ | 114.7 | P | 88.4 | P | 321.4 | P |
| | | ug = Mm 89481/len = 96/STRA = for | | | | | | |
| 6 | 103808_at | Cluster Incl AB016784 Mus musculus ref-11 mRNA, complete cds/cds = (261,2450)/gb = AB016784/gi = 3551508/ | 228.4 | P | 339.8 | P | 239.4 | P |
| | | ug = Mm 20885/len = 2829/STRA = for | | | | | | |
| 6 | 98410_at | Cluster Incl AJ007972 Mus musculus mRNA for GTPI protein/cds = (16,1203)/gb = AJ1007972)/gi = 4158175/ug = Mm 33902/ | 142 | A | 248.3 | P | 317.8 | P |
| | | len = 3338/STRA = for | | | | | | |
| 6 | 101465_at | Cluster Incl U06924 Signal transducer and activator of transcription 1/cds = (4,2253)/gb = U06924/gi = 458709/ug = Mm 8249/ | 1.8 | A | 80.1 | P | 104.5 | P |
| | | len = 2277/STRA = for | | | | | | |
| 6 | 101923_at | Cluster Incl U34277 Mus musculus PAF acetylhydrolase mRNA, complete cds/cds = (116,1438)/gb = U34277/gi = 1041235/ | 117 | P | 288.2 | P | 209.2 | P |
| | | ug = Mm 9277/len = 1507/STRA = for | | | | | | |
| 6 | 102641_at | Cluster Incl L03215: SFFV proviral integration 1/cds = UNKNOWN/gb = L03215/gi = 201035/ug = Mm.1302/len = 1232/ | 269.2 | P | 309.8 | P | 287.5 | P |
| | | STRA = for | | | | | | |
| 6 | 102706_l_at | Cluster Incl X61597 M musculus gene for kallikrein-binding protein/cds = (111,1364)/gb = X61597/gi = 52808/ug = Mm 14191/ | 13.5 | A | 48.5 | A | 46.2 | P |
| | | len = 1690/STRA = for | | | | | | |
| 6 | 102707_f_at | Cluster Incl X61597 M musculus gene for kallikrein-binding protein/cds = (111,1364)/gb = X61597/gi = 52808/ug = Mm 14191/ | 115.7 | P | 194.2 | P | 171.4 | P |
| | | len = 1690/STRA = for | | | | | | |
| 6 | 104202_at | Cluster Incl AI850624 UI-M-BG1-alk-d-02-0-UI s1 Mus musculus cDNA, 3 end/clone = UI-M-BG1-alk-d-02-0-UI/ | 230.3 | P | 291.8 | A | 372.7 | A |
| | | clone_end = 3/gb = AI850624/gi = 5494530/ug = Mm 27539/len = 416/STRA = rev | | | | | | |
| 6 | 104456_at | Cluster Incl AA863742 vx08b05 r1 Mus musculus cDNA, 5 end/clone = IMAGE-1263825/clone_end = 5/gb = AA863742/ | 84.4 | P | 100.1 | P | 85.8 | P |
| | | gi = 2956221/ug = Mm 29514/len = 402/STRA = for | | | | | | |
| 6 | 104677_at | Cluster Incl AI852008: UI-M-BH0-aiq-f-08-0-UI.s1 Mus musculus cDNA, 3 end/clone = UI-M-BH0-aiq-f-08-0-UI/ | 126.7 | P | 88.7 | P | 187.4 | P |
| | | clone_end = 3/gb = AI852008/gi = 5495914/ug = Mm 32542/len = 410/STRA = for | | | | | | |
| 6 | 92251_f_at | Cluster Incl AA960657 ub60c10 s1 Mus musculus cDNA, 3 end/clone = IMAGE-1382130/clone_end = 3/gb = AA960657/ | 96.7 | P | 256.5 | P | 305.9 | P |
| | | gi = 3126557/ug = Mm 35110/len = 467/STRA = rev | | | | | | |
| 6 | 93339_at | Cluster Incl AI846243 UI-M-AK1-aez-a-10-0-UI s1 Mus musculus cDNA, 3 end/clone = UI-M-AK1-aez-a-10-0-UI/ | 184.5 | P | 424.3 | P | 337 | A |
| | | clone_end = 3/gb = AI846243/gi = 5490149/ug = Mm 18706/len = 334/STRA = for | | | | | | |
| 6 | 99663_g_at | Cluster Incl AI194767: ul56d10 x1 Mus musculus cDNA, 3 end/clone = IMAGE-1886419/clone_end = 3/gb = AI194767/ | 149.3 | M | 277.3 | A | 236.8 | P |
| | | gi = 3747373/ug = Mm 4379/len = 525/STRA = for | | | | | | |
| 6 | 101588_at | Cluster Incl AF058055: Mus musculus monocarboxylate transpoter 1 (mMCT1) mRNA, complete cds/cds = (189,1670)/ | 15.2 | A | 25.7 | P | 35.2 | P |
| | | gb = AF058055/gi = 3047399/ug = Mm 9086/len = 3184/STRA = for | | | | | | |
| 6 | 102027_s_at | Cluster Incl AA204010 mu59g02 r1 Mus musculus cDNA, 5 end/clone = IMAGE-643730/clone_end = 5/gb = AA204010/ | 160.7 | P | −261.3 | P | 57 | P |
| | | gi = 1800701/ug = Mm 10124/len = 304/STRA = for | | | | | | |
| 6 | 103016_s_at | Cluster Incl X68273: CD68 antigen/cds = (91,1071)/gb = X68273/gi = 52988/ug = Mm 15819/len = 1234/STRA = for | 83.5 | A | 73.9 | P | 142.1 | P |
| 6 | 103397_at | Cluster Incl AA795486 vq95e08 r1 Mus musculus cDNA, 5 end/clone = IMAGE-1110086/clone_end = 5/gb = AA795486/ | 81.7 | A | 58.2 | P | 83.3 | P |
| | | gi = 2858441/ug = Mm 18713/len = 273/STRA = for | | | | | | |
| 6 | 95543_at | Cluster Incl AI843046 UI-M-AK1-aeq-g-04-0-UI s1 Mus musculus cDNA, 3 end/clone = UI-M-AK1-aeq-g-04-0-UI/ | 301.9 | P | 324.5 | P | 228.1 | P |
| | | clone_end = 3/gb = AI843046/gi = 5477259/ug = Mm 27685/len = 432/STRA = rev | | | | | | |
| 6 | 97346_at | Cluster Incl AW124115: UI-M-BH2.1-apr-b-11-0-UI s1 Mus musculus cDNA, 3 end/clone = UI-M-BH2.1-apr-b-11-0-UI/ | 103.9 | P | 163.9 | P | 80.4 | A |
| | | clone_end = 3/gb = AW124115/gi = $$/ug = Mm $$/len = $$/STRA = for | | | | | | |
| 6 | 98428_at | Cluster Incl AJ246002: Mus musculus mRNA for spastin protein orthologue (Spast gene)/cds = (0,1516)/gb = AJ246002/ | 34.3 | A | 26.3 | A | 4.5 | P |
| | | gi = 6273571/ug = Mm 34234/len = 1689/STRA = for | | | | | | |
| 6 | 101160_at | Cluster Incl X53798: Macrophage inflammatory protein 2/cds = (46,348)/gb = X53798/gi = 53128/ug = Mm 4979/len = 1083/ | 299.9 | P | 102.3 | P | 76.2 | P |
| | | STRA = for | | | | | | |
| 6 | 96031_r_at | Cluster Incl AW259411. up29c03.x1 Mus musculus cDNA, 3 end/clone = IMAGE-2655748/clone_end = 3/gb = AW259411/ | 34.8 | A | 50 | A | 81.9 | P |
| | | gi = 6632465/ug = Mm 25794/len = 465/STRA = for | | | | | | |
| 6 | 95752_at | Cluster Incl AI837369.UI-M-AP0-abm-a-02-0-UI s1 Mus musculus cDNA, 3 end/clone = UI-M-AP0-abm-a-02-0-UI/ | 208.6 | P | 39.4 | M | 327.5 | P |
| | | clone_end = 3/gb = AI837369/gi = 5471582/ug = Mm 29784/len = 371/STRA = for | | | | | | |
| 6 | 97507_at | Cluster Incl X67809 Peptidylprotyl isomerase C-associated protein/cds = (168,1901)/gb = X67809/gi = 297032/ug = Mm 3152/ | −26.8 | A | 13.2 | P | 314.6 | P |
| | | len = 2168/STRA = for | | | | | | |
| 6 | 102313_at | Cluster Incl L09737 GTP cyclohydrolase 1/cds = (122,847)/gb = L09737/gi = 293356/ug = Mm. 10651/len = 994/STRA = for | 60.2 | P | 104.5 | P | 60.8 | A |
| 6 | 103805_g_at | Cluster Incl AW231026: uo70a01.y1 Mus musculus cDNA, 5 end/clone = IMAGE-2647848/clone_end = 5/gb = AW231026/ | 114.9 | A | 160 | P | 148.4 | P |
| | | gi = 6580258/ug = Mm 20156/len = 482/STRA = for | | | | | | |
| 6 | 103887_at | Cluster Incl M83219 Mus musculus intracellular calcium-binding protein (MRP14) mRNA, complete cds/cds = (33,374)/ | −11.1 | A | 843.2 | P | 1402.9 | P |
| | | gb = M83219/gi = 199807/ug = Mm 2128/len = 488/STRA = for | | | | | | |
| 6 | 104528_at | Cluster Incl AI181549: uc59h04 r1 Mus musculus cDNA, 5 end/clone = IMAGE-1429975/clone_end = 5/gb = AI181549/ | −19.4 | A | 22.1 | M | 14.7 | P |
| | | gi = 3732187/ug = Mm 2310/len = 435/STRA = for | | | | | | |
| 6 | 104549_at | Cluster Incl AI844761: UI-M-AL1-ahq-e-12-0-UI s1 Mus musculus cDNA, 3 end/clone = UI-M-AL1-ahq-e-12-0-UI/ | 102.7 | A | 144.2 | A | 274.6 | P |
| | | clone_end = 3/gb = AI844761/gi = 5488667/ug = Mm 23702/len = 384/STRA = for | | | | | | |
| 6 | 104606_at | Cluster Incl M55561 CD80 antigen/cds = (78,302)/gb = M55561/gi = 200225/ug = Mm 24130/len = 491/STRA = for | 321.3 | A | 400 | P | 279.5 | M |
| 6 | 96188_at | Cluster Incl AF052506 Mus musculus double-stranded RNA-specific adenosine deaminase mRNA, complete cds/ | 171.2 | A | 112.9 | P | 199 | P |
| | | cds = (6,3484)/gb = AF052506/gi = 2981096/ug = Mm 28502/len = 3466/STRA = for | | | | | | |
| 6 | 97409_at | Cluster Incl U19119 Mus musculus G-protein-like LRG-47 mRNA, complete cds/cds = (445,1674)/gb = U19119/gi = 633753/ | 76.2 | A | 18 | P | 182.2 | P |
| | | ug = Mm 29938/len = 2227/STRA = for | | | | | | |
| 6 | 98500_at | Cluster Incl D13695 Lymphocyte antigen 84/cds = (42,1745)/gb = D13695/gi = 286100/ug = Mm 35692/len = 4989/STRA = for | 32.1 | A | 28.7 | A | 56.6 | P |
| 6 | 103295_at | Cluster Incl AI854482 UI-M-BH0-ait-h-07-0-UI s1 Mus musculus cDNA, 3 end/clone = UI-M-BH0-ait-h-07-0-UI/clone_end = 3/ | 226.3 | A | 256 | P | 297.7 | P |
| | | gb = AI854482/gi = 5498388/ug = Mm 20971/len = 389/STRA = for | | | | | | |
| 6 | 101681_f_at | Cluster Incl U21906 Histocompatibility 2, blastocyst/cds = (236,1285)/gb = U21906/gi = 2326165/ug = Mm 34289/len = 1515/ | 395 | P | 463.2 | A | 533.9 | P |
| | | STRA = for | | | | | | |
| 6 | 92811_at | Cluster Incl X74154 Retinol binding protein 2, cellular/cds = (57,461)/gb = X74154/gi = 397351/ug = Mm 12825/len = 479/ | 147.6 | P | 43.4 | P | 205.3 | A |
| | | STRA = for | | | | | | |
| 6 | 95505_at | Cluster Incl AW060509 UI-M-BH1-ann-c-12-0-UI.s1 Mus musculus cDNA, 3 end/clone = UI-M-BH1-ann-c-12-0-UI/ | 54.9 | P | 19.5 | A | 30.7 | M |
| | | clone_end = 3/gb = AW060509/gi = 6008260/ug = Mm.25187/len = 308/STRA = for | | | | | | |
| 6 | 98045_s_at | Cluster Incl U18869: Mus musculus mitogen-responsive 96 kDa phosphoprotein p96 mRNA, alternatively spliced p67 | 38.4 | M | −55.7 | A | −19.3 | A |
| | | mRNA, and alternatively spliced p93 mRNA, complete cds/cds = (216,2516)/gb = U18869/gi = 1176369/ug = Mm 34248/ | | | | | | |
| | | len = 3347/STRA = for | | | | | | |
| 6 | 98562_at | Cluster Incl X58861: Complement component 1, q subcomponent, alpha polypeptide/cds = (29,766)/gb = X58861/gi = 50226/ | −132.5 | A | 87.3 | A | −65.5 | A |
| | | ug = Mm.370/len = 1033/STRA = for | | | | | | |
| 6 | 100611_at | Cluster Incl M21050 Mouse lysozyme M gene/cds = (30,476)/gb = M21050/gi = 198950/ug = Mm 654/len = 1264/STRA = for | −25.3 | A | −84.2 | A | −49.6 | A |
| 6 | 102351_at | Cluster Incl J05118: Carboxypeptidase A3, mast cell/cds = (6,1259)/gb = J05118/gi = 192372/ug = Mm 1135/len = 1442/ | 113.5 | A | 61.4 | A | 58.8 | A |
| | | STRA = rev | | | | | | |
| 6 | 102791_at | Cluster Incl U22033 Large multifunctional protease 7/cds = (0,830)/gb = U22033/gi = 972985/ug = Mm 13913/len = 831/ | 186.1 | A | 174.2 | P | 212.6 | P |
| | | STRA = for | | | | | | |
| 6 | 104728_at | Cluster Incl L27439 Mus musculus protein S mRNA, partial cds/cds = (0,1933)/gb = L27439/gi = 487866/ug = Mm 24611/ | 93.5 | A | 113.2 | P | 120.1 | P |
| | | len = 3079/STRA = for | | | | | | |
| 6 | 98465_f_at | Cluster Incl M31419 Interferon activated gene 204/cds = (127,2049)/gb = M31419/gi = 198399/ug = Mm 34817/len = 2353/ | −29.9 | A | 172 | P | 195.8 | P |
| | | STRA = for | | | | | | |
| 6 | 100397_at | Cluster Incl AF024637 TYRO protein tyrosine kinase binding protein/cds = (0,344)/gb = AF024637/gi = 2906001/ | 161.4 | A | 126.7 | A | 135.1 | A |
| | | ug = Mm 46301/len = 345/STRA = for | | | | | | |
| 6 | 102264_at | Cluster Incl AF099972 Mus musculus schlafen1 (Slfn1) mRNA, complete cds/cds = (257,1270)/gb = AF099972/gi = 3983147/ | 236.6 | P | 218.4 | A | 193.4 | A |
| | | ug = Mm 10948/len = 1881/STRA = for | | | | | | |
| 6 | 103210_at | Cluster Incl M29855 Interleukin 3 receptor, beta chain 2/cds = (175,2811)/gb = M29855/gi = 198342/ug = Mm 1940/len = 4613/ | 13.5 | A | 131.7 | P | 84.6 | A |
| | | STRA = for | | | | | | |
| 6 | 103226_at | Cluster Incl Z11974 Mannose receptor, C type 1/cds = (99,4469)/gb = Z11974/gi = 52997/ug = Mm 2019/len = 5085/ | −8.1 | A | −36.1 | A | −15.7 | A |
| | | STRA = for | | | | | | |
| 6 | 103707_at | Cluster Incl U77461 Complement component 3a receptor 1/cds = (0,1433)/gb = U77461/gi = 2130534/ug = Mm 2408/ | 119 | A | 94.6 | A | 112.2 | A |
| | | len = 1434/STRA = for | | | | | | |
| 6 | 92223_at | Cluster Incl X66295: Complement component 1, q subcomponent, c polypeptide/cds = (38,778)/gb = X66295/gi = 50228/ | −9.4 | A | −31.2 | A | 11.5 | A |
| | | ug = Mm.3453/len = 1019/STRA = for | | | | | | |
| 6 | 93622_at | Cluster Incl M57626 Mast cell protease 6/cds = (33,863)/gb = M57626/gi = 200508/ug = Mm.7409/len = 1108/STRA = for | 370.9 | A | 286.1 | A | 258.4 | A |
| 6 | 99413_at | Cluster Incl U29678 Chemokine (C-C) receptor 1/cds = (0,1067)/gb = U29678/gi = 1109785/ug = Mm 57051/len = 1068/ | −3.4 | A | 6.9 | A | 20.9 | P |
| | | STRA = for | | | | | | |
| 6 | 100328_s_at | Cluster Incl U96684 Paired-lg-like receptor A3/cds = (10,2001)/gb = U96684/gi = 2138356/ug = Mm 87801/len = 2101/ | 123.3 | A | 34.9 | A | 102.8 | A |
| | | STRA = for | | | | | | |
| 6 | 95806_f_at | Cluster Incl M68899 Mouse mast cell chymase 2 mRNA, complete cds/cds = (120,650)/gb = M68899/gi = 199030/ | −0.2 | A | 41.4 | A | 15.7 | A |
| | | ug = Mm 87865/len = 901/STRA = for | | | | | | |
| 6 | 102104_f_at | Cluster Incl AI504305 vl04g11 x1 Mus musculus cDNA, 3 end/clone = IMAGE-963236/clone_end = 3/gb = AI504305/ | −12.4 | A | 36.6 | A | −4.9 | A |
| | | gi = 4402156/ug = Mm.42485/len = 580/STRA = rev | | | | | | |
| 6 | 95159_at | Cluster Incl AI846849: UI-M-AI1-afk-e-04-0-UI.s1 Mus musculus cDNA, 3 end/clone = UI-M-AI1-afk-o-04-0-UI/clone_end = 3/ | 38.2 | A | 65.4 | A | 111.8 | P |
| | | gb = AI846849/gi = 5490755/ug = Mm.29484/len = 332/STRA = for | | | | | | |
| 6 | 96297_at | Cluster Incl AI845934: UI-M-AK1-aex-f-03-0-UI.s1 Mus musculus cDNA, 3 end/clone = UI-M-AK1-aex-f-03-0-UI/ | 1.1 | A | 1.7 | A | 40.9 | A |
| | | clone_end = 3/gb = AI845934/gi = 5489840/ug = Mm.29906/len = 280/STRA = for | | | | | | |
| 6 | 97530_at | Cluster Incl U82627: Mus musculus ubiquitin-conjugating enzyme mE2 mRNA, complete cds/cds = (105,581)/gb = U82627/ | 23.6 | A | 68.2 | A | 141 | P |
| | | gi = 1857450/ug = Mm 3268/len = 769/STRA = for | | | | | | |
| 6 | 100100_at | Cluster Incl AA762212-vv47h06 r1 Mus musculus cDNA, 5 end/clone = IMAGE-1225595/clone_end = 5/gb = AA762212/ | 36.9 | A | 65.5 | A | 60.9 | A |
| | | gi = 2811959/ug = Mm 46316/len = 509/STRA = for | | | | | | |
| 6 | 102021_at | Cluster Incl M27960 Mouse interleukin-4 receptor (secreted form) mRNA, complete cds/cds = (236,928)/gb = M27960/ | 89.2 | A | 118.3 | A | 208.4 | P |
| | | gi = 198365/ug = Mm 1009/len = 3697/STRA = for | | | | | | |
| 6 | 102353_at | Cluster Incl M31039 Mus musculus complement receptor 3 beta subunit MAC-1/mRNA, complete cds/cds = (0,2315)/ | 20.9 | A | −64.5 | A | −27 | A |
| | | gb = M31039/gi = 198434/ug = Mm 1137/len = 2459/STRA = for | | | | | | |
| 6 | 103394_at | Cluster Incl U72680 Mus musculus ion channel homolog RIC mRNA, complete cds/cds = (48,584)/gb = U72680/gi = 1872490/ | 77.1 | A | −68.7 | A | 69.9 | A |
| | | ug = Mm 1870/len = 797/STRA = for | | | | | | |
| 6 | 96772_at | Cluster Incl J04620 Mouse primase p49 subunit (prlA) mRNA, complete cds/cds = (73,1326)/gb = J04620/gi = 200196/ | 163.3 | A | 149.9 | A | 152.7 | A |
| | | ug = Mm 2903/len = 1507/STRA = for | | | | | | |
| 6 | 99979_at | Cluster Incl X78445 M musculus Cyp1-b-1 mRNA for cytochrome P450/cds = (179,1810)/gb = X78445/gi = 506463/ | −43.1 | A | −3.8 | P | 6.8 | P |
| | | ug = Mm 4443/len = 2006/STRA = for | | | | | | |
| 6 | 101392_at | Cluster Incl AW122839 UI-M-BH2 1-aoz-e-09-0-UI.s1 Mus musculus cDNA, 3 end/clone = UI-M-BH2.1-aoz-e-09-0-UI/ | 122.6 | P | 80.9 | A | 156.6 | A |
| | | clone_end = 3/gb = AW122839/gi = 6098369/ug = Mm 7196/len = 382/STRA = for | | | | | | |
| 6 | 102292_at | Cluster Incl U00937: Mus musculus GADD45 protein (gadd45) gene, complete cds/cds = (0,497)/gb = U00937/gi = 392933/ | −79.5 | A | 24.9 | A | 58.8 | A |
| | | ug = Mm 1236/len = 498/STRA = for | | | | | | |
| 6 | 104006_at | Cluster Incl L21768 Mus musculus eps15 mRNA, complete cds/cds = (110,2803)/gb = L21768/gi = 404756/ug = Mm 267/ | 68.8 | A | 80.6 | A | 146 | A |
| | | len = 3033/STRA = for | | | | | | |
| 6 | 104657_at | Cluster Incl D26177.Leukemia Inhibitory factor receptor/cds = (71,3349)/gb = D26177/gi = 473718/ug = Mm.3174/len = 3399/ | −5.6 | P | −43.5 | A | −18.9 | A |
| | | STRA = for | | | | | | |
| 6 | 92443_I_at | Cluster Incl X16493 Zinc finger protein 1/cds = (320,1594)/gb = X16493/gi = 55482/ug = Mm 4184/len = 1939/STRA = rev | 68.3 | A | −53.7 | P | 53.7 | P |
| 6 | 92472_f_at | Cluster Incl AF099973 Mus musculus schlafen2 (Slfn2) mRNA, complete cds/cds = (182,1318)/gb = AF099973/gi = 3983149/ | 47.1 | A | 51.1 | A | 71.5 | A |
| | | ug = Mm 42124/len = 1684/STRA = for | | | | | | |
| 6 | 92718_at | Cluster Incl AI158810 ud38d09 r1 Mus musculus cDNA, 5 end/clone = IMAGE-1448177/clone_end = 5/gb = AI158810/ | −58.3 | A | 43.8 | A | 34.7 | A |
| | | gi = 3691992/ug = Mm 48382/len = 251/STRA = for | | | | | | |
| 6 | 94774_at | Cluster Incl M31416 Interferon activated gene 202/cds = (293,1830)/gb = M31418/gi = 198396/ug = Mm.16744/len = 1857/ | −17.2 | A | 213.5 | P | 138.5 | P |
| | | STRA = for | | | | | | |
| 6 | 98822_at | Cluster Incl X56602 Mus musculus mRNA Interferon-induced 15-KDa protein/cds = (111,593)/gb = X58602/gi = 52737/ | −143.6 | A | 137.9 | P | 706.5 | P |
| | | ug = Mm 4950/len = 728/STRA = for | | | | | | |
| 6 | 97013_f_at | Cluster Incl AW046124 UI-M-BH1-alf-e-03-0-UI s1 Mus musculus cDNA, 3 end/clone = UI-M-BH1-alf-e-03-0-Ul/ | 286.4 | M | 233.4 | A | 127.6 | A |
| | | clone_end = 3/gb = AW046124/gi = 5906653/ug = Mm.88950/len = 249/STRA = rev | | | | | | |
| 6 | 101635_f_at | Cluster Incl M16360 Mouse major unnary protein V (MUP V) mRNA, complete cds/cds = (58,600)/gb = M16360/gi = 199884/ | −7.8 | A | 14.8 | A | 93.2 | P |
| | | ug = Mm 30274/len = 872/STRA = for | | | | | | |
| 6 | 101682_f_at | Cluster Incl M16358 Major unnary protein 4/cds = (66,602)/gb = M16358/gi = 199882/ug = Mm 34335/len = 875/STRA = for | 61 | A | 58.8 | A | 79.5 | A |
| 7 | 92539_at | Cluster Incl M16465 Calpactin I light chain/cds = (68,361)/gb = M16465/gi = 192360/ug = Mm.1/len = 600/STRA = for | 3998.7 | P | 2770.3 | P | 3553.5 | P |
| 7 | 92603_at | Cluster Incl U13840: Mus musculus vacuolar adenosine triphosphatase subunit D mRNA, complete cds/cds = (17,1072)/ | 1029.5 | P | 739.8 | P | 840.9 | P |
| | | gb = U13840/gi = 3955099/ug = Mm 1081/len = 1509/STRA = for | | | | | | |
| 7 | 92607_at | Cluster Incl AF017994.Mus musculus Peg1/MEST protein (Peg1/MEST) gene, partial cds/cds = (247,427)/gb = AF017994/ | 1820.6 | P | 757.5 | P | 1132.6 | P |
| | | gi = 2570405/ug = Mm.1089/len = 428/STRA = for | | | | | | |
| 7 | 92638_at | Cluster Incl Z67745 M. musculus mRNA for phosphatase 2A catalytic subunit, Isotype alpha/cds = (0,929)/gb = Z67745/ | 624 | P | 453.7 | P | 406.6 | P |
| | | gi = 1246794/ug = Mm.11711/len = 930/STRA = for | | | | | | |
| 7 | 92855_at | Cluster Incl Z50159 M musculus mRNA for Sui1/cds = (0,230)/gb = Z50159/gi = 927434/ug = Mm 13886/len = 355/STRA = for | 2167.7 | P | 1278.2 | P | 1449.4 | P |
| 7 | 93093_at | Cluster Incl U35623.Myeloid cell leukemia sequence 1/cds = (48,1043)/gb = U35623/gi = 2522268/ug = Mm 1639/len = 3396/ | 1271.4 | P | 934.7 | P | 1116.2 | P |
| | | STRA = for | | | | | | |
| 7 | 93100_at | Cluster Incl X13297 Mouse mRNA for vascular smooth muscle alpha-actin/cds = (49,1182)/gb = X13297/gi = 49861/ | 1483.9 | P | 508.6 | P | 665.6 | P |
| | | ug = Mm 16537/len = 1340/STRA = for | | | | | | |
| 7 | 93327_at | Cluster Incl AI842665 UI-M-AO1-aeo-a-02-0-UI.s1 Mus musculus cDNA, 3 end/clone = UI-M-AO1-aeo-a-02-0-UI/ | 173.3 | P | 156 | P | 192.2 | P |
| | | clone_end = 3/gb = AI842685/gi = 5476878/ug = Mm 186/len = 288/STRA = for | | | | | | |
| 7 | 93353_at | Cluster Incl AF013262: Lumican/cds = (109,1125)/gb = AF013262/gi = 2654773/ug = Mm 18888/len = 1782/STRA = for | 2236.6 | P | 1060.5 | P | 990.4 | P |
| 7 | 93517_at | Cluster Incl Z18272 Procollagen, type VI, alpha 2/cds = (0,1115)/gb = Z18272/gi = 57957/ug = Mm.1949/len = 1562/ | 3127.3 | P | 1413.7 | P | 1948.3 | P |
| | | STRA = for | | | | | | |
| 7 | 93743_at | Cluster Incl AI838388: UI-M-AJO-aav-f-02-0-UI.s2 Mus musculus cDNA, 3 end/clone = UI-M-AJ0-aav-f-02-0-UI/ | 186.5 | P | −16.3 | P | 130.3 | P |
| | | clone_end = 3/gb = AI838388/gi = 5472601/ug = Mm.21069/len = 414/STRA = for | | | | | | |
| 7 | 93793_at | Cluster Incl AW122780 UI-M-BH2.1-aoy-c-12-0-UI s1 Mus musculus cDNA, 3 end/clone = UI-M-BH2 1-aoy-c-12-0-UI/ | 956 | P | 730.2 | P | 704.1 | P |
| | | clone_end = 3/gb = AW122780/gi = 6098310/ug = Mm.21387/len = 148/STRA = for | | | | | | |
| 7 | 93975_at | Cluster Incl AI853531 UI-M-BH0-ajd-f-01-0-UI s1 Mus musculus cDNA, 3 end/clone = UI-M-BH0-ajd-f-01-0-UI/ | 697.5 | P | 490.8 | P | 643.8 | P |
| | | clone_end = 3/gb = AI853531/gi = 5497437/ug = Mm 21679/len = 425/STRA = for | | | | | | |
| 7 | 94060_at | Cluster Incl AI852623 UI-M-BH0-aiu-e-12-0-UI.s1 Mus musculus cDNA, 3 end/clone = UI-M-BH0-alu-e-12-0-UI/ | 532 | P | 439.2 | P | 462.2 | P |
| | | clone_end = 3/gb = AI852623/gi = 5496529/ug = Mm.22044/len = 416/STRA = for | | | | | | |
| 7 | 94218_at | Cluster Incl AW121091.UI-M-BH2 3-aof-h-07-0-UI s1 Mus musculus cDNA, 3 end/clone = UI-M-BH2 3-aof-h-07-0-UI/ | 1966 | P | 1070.9 | P | 1089.7 | P |
| | | clone_end = 3/gb = AW121091/gi = 6096424/ug = Mm.22227/len = 214/STRA = for | | | | | | |
| 7 | 94238_at | Cluster Incl AW228316 up21f09 y1 Mus musculus cDNA, 5 end/clone = IMAGE-2655017/clone_end = 5/gb = AW228316/ | 247.7 | P | 191.4 | P | 178.8 | P |
| | | gi = 6557612/ug = Mm 22345/len = 193/STRA = for | | | | | | |
| 7 | 94246_at | Cluster Incl J04103: E26 avian leukemia oncogene 2,3 domain/cds = (137,1543)/gb = J04103/gi = 193193/ug = Mm.22365/ | 1047.5 | P | 774.8 | P | 912.8 | P |
| | | len = 1552/STRA = for | | | | | | |
| 7 | 94256_at | Cluster Incl AI849533 UI-M-AH1-agt-h-01-0-UI.s1 Mus musculus cDNA, 3 end/clone = UI-M-AH1-agt-h-01-0-UI/ | 1317.7 | P | 1489.3 | P | 1187.9 | P |
| | | clone_end = 3/gb = AI849533/gi = 5493439/ug = Mm 22409/len = 360/STRA = for | | | | | | |
| 7 | 94258_at | Cluster Incl L07918 Mus musculus GDP-dissociation inhibitor mRNA, preferentially expressed in hematopoietic cells, | 1324.2 | P | 986.4 | P | 1061.8 | P |
| | | complete cds/cds = (32,634)/gb = L07918/gi = 193461/ug = Mm 2241/len = 1099/STRA = rev | | | | | | |
| 7 | 94301_at | Cluster Incl AI843269.UI-M-AO1-aei-c-08-0-UI s1 Mus musculus cDNA, 3 end/clone = UI-M-AO1-aei-c-08-0-UI/ | 742.6 | P | 464.3 | P | 562.4 | P |
| | | clone_end = 3/gb = AI843269/gi = 5477482/ug = Mm 22602/len = 310/STRA = for | | | | | | |
| 7 | 94303_at | Cluster Incl U11274 Mus musculus clone pmuAUF1-3 RNA-binding protein AUF1 mRNA, complete cds/cds = (142,951)/ | 1060.8 | P | 859.5 | P | 729.5 | P |
| | | gb = U11274/gi = 508269/ug = Mm 22617/len = 1322/STRA = for | | | | | | |
| 7 | 95405_at | Cluster Incl AW045534: UI-M-BH1-aks-c-09-0-UI s1 Mus musculus cDNA, 3 end/clone = UI-M-BH1-aks-c-09-0-UI/ | 331.9 | P | 218.3 | P | 243.9 | P |
| | | clone_end = 3/gb = AW045534/gi = 5906063/ug = Mm 24209/len = 435/STRA = for | | | | | | |
| 7 | 95413_at | Cluster Incl AI851332 UI-M-BH0-akh-b-07-0-UI s1 Mus musculus cDNA, 3 end/clone = UI-M-BH0-akh-b-07-0-UI/ | 1103.8 | P | 805.3 | P | 924.5 | P |
| | | clone_end = 3/gb = AI851332/gi = 5495238/ug = Mm 24272/len = 387/STRA = for | | | | | | |
| 7 | 95477_at | Cluster Incl AW125185 UI-M-BH2 1-apz-g-10-0-UI s1 Mus musculus cDNA, 3 end/clone = UI-M-BH2.1-apz-g-10-0-UI/ | 1372.1 | P | 1208.1 | P | 1206.6 | P |
| | | clone_end = 3/gb = AW125185/gi = 6100715/ug = Mm.24847/len = 215/STRA = for | | | | | | |
| 7 | 96047_at | Cluster Incl U63146 Mus musculus retinol binding protein (RBP) mRNA, complete cds/cds = (0,605)/gb = U63146/ | 290.3 | P | 143.3 | P | 244 | P |
| | | gi = 1515449/ug = Mm 2605/len = 606/STRA = for | | | | | | |
| 7 | 96083_s_at | Cluster Incl AI226368 uf07b12.y1 Mus musculus cDNA, 5 end/clone = IMAGE-1510655/clone_end = 5/gb = AI226368/ | 1352.3 | P | 889.9 | P | 861.1 | P |
| | | gi = 3809421/ug = Mm 26617/len = 340/STRA = for | | | | | | |
| 7 | 96084_at | Cluster Incl AB017020 Mus musculus mRNA for JKTBP, complete cds/cds = (123,1028)/gb = AB017020/gi = 4514630/ | 520.5 | P | 214.8 | P | 304.9 | P |
| | | ug = Mm 26617/len = 1307/STRA = for | | | | | | |
| 7 | 96633_s_at | Cluster Incl AA529583 vi41d07 r1 Mus musculus cDNA, 5 end/clone = IMAGE-906349/clone_end = 5/gb = AA529583/ | 1429.6 | P | 1201.5 | P | 990.2 | P |
| | | gi = 2272289/ug = Mm 27218/len = 546/STRA = for | | | | | | |
| 7 | 96664_at | Cluster Incl AB025966 Mus musculus mRNA for KIAA312p, partial cds/cds = (0,1123)/gb = AB025966/gi = 5931572/ | 1049 | P | 843.6 | P | 851.1 | P |
| | | ug = Mm 27372/len = 1638/STRA = for | | | | | | |
| 7 | 97211_at | Cluster Incl AI747444: ul15f07.x1 Mus musculus cDNA, 3 end/clone = IMAGE-2076901/clone_end = 3/gb = AI747444/ | 576.6 | P | 349.5 | P | 329.6 | P |
| | | gi = 5125708/ug = Mm 27714/len = 657/STRA = for | | | | | | |
| 7 | 97240_g_at | Cluster Incl AI845106.UI-M-BG0-ahs-a-04-0-UI s1 Mus musculus cDNA, 3 end/clone = UI-M-BG0-ahs-a-04-0-UI/ | 1209.2 | P | 1061.3 | P | 1196.4 | P |
| | | clone_end = 3/gb = AI845106/gi = 5489012/ug = Mm 27839/len = 141/STRA = for | | | | | | |
| 7 | 97819_at | Cluster Incl AI843119 UI-M-AK1-aes-f-05-0-UI s1 Mus musculus cDNA, 3 end/clone = UI-M-AK1-aes-f-05-0-UI/ | 1009.2 | P | 388.3 | P | 698.2 | P |
| | | clone_end = 3/gb = AI843119/gi = 5477332/ug = Mm 282/len = 199/STRA = for | | | | | | |
| 7 | 97864_at | Cluster Incl AW258842 um74d01 y1 Mus musculus cDNA, 5 end/clone = IMAGE-2300929/clone_end = 5/gb = AW258842/ | 237.4 | P | 266.9 | P | 205.2 | P |
| | | gi = 6631823/ug = Mm 28327/len = 321/STRA = for | | | | | | |
| 7 | 97865_g_at | Cluster Incl AW258842 um74d01 y1 Mus musculus cDNA, 5 end/clone = IMAGE-2300929/clone_end = 5/gb = AW258842/ | 737.5 | P | 592.2 | P | 596.5 | P |
| | | gi = 6631823/ug = Mm 28327/len = 321/STRA = for | | | | | | |
| 7 | 98937_at | Cluster Incl AW049795 UI-M-BH1-anm-1-07-0-UI s1 Mus musculus cDNA, 3 end/clone = UI-M-BH1-anm-f-07-0-UI/ | 1190.5 | P | 796.3 | P | 1057.4 | P |
| | | clone_end = 3/gb = AW049795/gi = 5910324/ug = Mm 28689/len = 562/STRA = for | | | | | | |
| 7 | 94448_at | Cluster Incl AJ006289: Mus musculus mRNA for bcl-10 protein/cds = (78,779)/gb = AJ006289/gi = 4049471/ug = Mm.28782/ | 1048.4 | P | 451.4 | P | 524.1 | P |
| | | len = 1756/STRA = for | | | | | | |
| 7 | 94516_f_at | Cluster Incl M55181: Preproenkephalin 2/cds = UNKNOWN/gb = M55181/gi = 201032/ug = Mm.2899/len = 1408/STRA = for | 3108.4 | P | 1515.6 | P | 3388 | P |
| 7 | 95709_at | Cluster Incl AW012491 uo05f02 y1 Mus musculus cDNA, 5 end/clone = IMAGE-2582523/clone_end = 5/gb = AW012491/ | 1355.4 | P | 1004.1 | P | 995.3 | P |
| | | gi = 5861269/ug = Mm 29703/len = 528/STRA = for | | | | | | |
| 7 | 96254_at | Cluster Incl AB028272 Mus musculus hsp40 mRNA for heat shock protein 40, complete cds/cds = (139,1161)/ | 491.1 | P | 289.9 | P | 309.8 | P |
| | | gb = AB028272/gi = 6531981/ug = Mm 2982/len = 2279/STRA = for | | | | | | |
| 7 | 96271_at | Cluster Incl AA914105 vy92c01.r1 Mus musculus cDNA, 5 end/clone = IMAGE-1313664/clone_end = 5/gb = AA914105/ | 1547.3 | P | 1578.6 | P | 1449.6 | P |
| | | gi = 3053497/ug = Mm 29853/len = 415/STRA = for | | | | | | |
| 7 | 96288_at | Cluster Incl AI648831 uk29f08.x1 Mus musculus cDNA, 3 end/clone = IMAGE-1970439/clone_end = 3/gb = AI648831/ | 411.9 | P | 385.4 | P | 395.4 | P |
| | | gi = 4729665/ug = Mm 29889/len = 526/STRA = for | | | | | | |
| 7 | 96325_at | Cluster Incl AW124874 UI-M-BH2.1-apu-b-08-0-UI s1 Mus musculus cDNA, 3 end/clone = UI-M-BH2 1-apu-b-08-0-UI/ | 274.7 | P | 208.3 | P | 257.5 | P |
| | | clone_end = 3/gb = AW124874/gi = 6100404/ug = Mm 29949/len = 429/STRA = for | | | | | | |
| 7 | 96336_at | Cluster Incl AI844626 UI-M-AL1-ahr-a-04-0-UI.s1 Mus musculus cDNA, 3 end/clone = UI-M-AL1-ahr-a-04-0-UI/ | 872.1 | P | 315.9 | P | 529 | P |
| | | clone_end = 3/gb = AI844626/gi = 5488532/ug = Mm.29975/len = 568/STRA = for | | | | | | |
| 7 | 96841_at | Cluster Incl AW046627 UI-M-BH1-ald-c-09-0-UI s1 Mus musculus cDNA, 3 end/clone = UI-M-BH1-ald-c-09-0-UI/ | 717.9 | P | 325.3 | P | 497.4 | P |
| | | clone_end = 3/gb = AW046627/gi = 5907158/ug = Mm 30018/len = 268/STRA = for | | | | | | |
| 7 | 96920_at | Cluster Incl AW125478: UI-M-BH2.2-aqm-c-03-0-UI s1 Mus musculus cDNA, 3 end/clone = UI-M-BH2 2-aqm-c-03-0-UI/ | 1736.8 | P | 863.4 | P | 955 | P |
| | | clone_end = 3/gb = AW125478/gi = 6101008/ug = Mm 30156/len = 468/STRA = for | | | | | | |
| 7 | 97450_s_at | Cluster Incl AA986258 uc74b03.y1 Mus musculus cDNA, 5 end/clone = IMAGE-1431341/clone_end = 5/gb = AA986258/ | 407.5 | P | 342.1 | P | 277.4 | P |
| | | gi = 3167582/ug = Mm 30250/len = 552/STRA = for | | | | | | |
| 7 | 97462_at | Cluster Incl AI847584 UI-M-AP1-agg-a-06-0-UI s1 Mus musculus cDNA, 3 end/clone = UI-M-AP1-agg-a-06-0-UI/ | 737.8 | P | 669.7 | P | 628.4 | P |
| | | clone_end = 3/gb = AI847584/gi = 5491490/ug = Mm 3035/len = 340/STRA = for | | | | | | |
| 7 | 97492_at | Cluster Incl AW121746: UI-M-BH2.3-aoe-c-09-0-UI s1 Mus musculus cDNA, 3 end/clone = UI-M-BH2 3-aoe-c-09-0-UI/ | 309.1 | P | 234 | P | 285 | P |
| | | clone_end = 3/gb = AW121746/gi = 6097081/ug = Mm 31076/len = 293/STRA = for | | | | | | |
| 7 | 97496_f_at | Cluster Incl AW048944.UI-M-BH1-amo-e-07-0-UI.s1 Mus musculus cDNA, 3 end/clone = UI-M-BHI-amo-e-07-0-UI/ | 1159.9 | P | 961.8 | P | 1179.9 | P |
| | | clone_end = 3/gb = AW048944/gi = 5909473/ug = Mm 3124/len = 296/STRA = for | | | | | | |
| 7 | 97538_at | Cluster Incl M19279 Beta-glucuronidase structural/cds = (12,1958)/gb = M19279/gi = 193524/ug = Mm 3317/len = 2454/ | 185.1 | P | 125 | P | 122.6 | P |
| | | STRA = for | | | | | | |
| 7 | 98084_at | Cluster Incl AI849834: UI-M-AL1-aho-h-09-0-UI s1 Mus musculus cDNA, 3 end/clone = UI-M-AL1-aho-h-09-0-UI/ | 1506.9 | P | 1266.7 | P | 1243.1 | P |
| | | clone_end = 3/gb = AI849834/gi = 5493780/ug = Mm 34577/len = 464/STRA = for | | | | | | |
| 7 | 98110_at | Cluster Incl AI853375: UI-M-BH0-air-e-09-0-UI s1 Mus musculus cDNA, 3 end/clone = UI-M-BH0-air-e-09-0-UI/ | 423.7 | P | 427 | P | 342.5 | P |
| | | clone_end = 3/gb = AI853375/gi = 5497281/ug = Mm 34810/len = 388/STRA = for | | | | | | |
| 7 | 98593_at | Cluster Incl AJ006215: Mus musculus mRNA for CMP-N-acetylneuraminic acid synthetase/cds = (0,1298)/gb = AJ006215/ | 335 | P | 233.9 | P | 245.2 | P |
| | | gi = 3413319/ug = Mm.3820/len = 1687/STRA = for | | | | | | |
| 7 | 99082_at | Cluster Incl L07063 Mus musculus FKBP65 binding protein mRNA, complete cds/cds = (93,1838)/gb = L07063/gi = 894161/ | 603.3 | P | 344.3 | P | 444.6 | P |
| | | ug = Mm 3894/len = 2597/STRA = for | | | | | | |
| 7 | 99127_at | Cluster Incl X61506 Mouse E46 mRNA for E46 protein/cds = (198,1478)/gb = X61506/gi = 50794/ug = Mm 4098/len = 1668/ | 1437.1 | P | 981.3 | P | 1044.9 | P |
| | | STRA = for | | | | | | |
| 7 | 99133_at | Cluster Incl X14309 Antigen identified by monoclonal antibodies 4F2/cds = (92,1672)/gb = X14309/gi = 49749/ug = Mm 4114/ | 1276.2 | P | 1157.5 | P | 1092.3 | P |
| | | len = 1828/STRA = for | | | | | | |
| 7 | 99563_at | Cluster Incl AW124681 UI-M-BH2.1-apw-g-06-0-UI s1 Mus musculus cDNA, 3 end/clone = UI-M-BH2 1-apw-g-06-0-UI/ | 575.9 | P | 478.9 | P | 527.2 | P |
| | | clone_end = 3/gb = AW124681/gi = 6100211/ug = Mm 42193/len = 421/STRA = for | | | | | | |
| 7 | 99602_at | Cluster Incl AF064088 Mus musculus transcription factor GIF mRNA, complete cds/cds = (109,1548)/gb = AF064088/ | 968.6 | P | 536.8 | P | 552.4 | P |
| | | gi = 3139151/ug = Mm 4292/len = 3036/STRA = for | | | | | | |
| 7 | 100094_at | Cluster Incl U88539 Mus musculus chromatin structural protein homolog Supt5hp (Supt5h) mRNA, complete cds/ | 499 | P | 559.5 | P | 580.7 | P |
| | | cds = (61,3309)/gb = U88539/gi = 2754751/ug = Mm 460/len = 3538/STRA = for | | | | | | |
| 7 | 100115_at | Cluster Incl AW124128 UI-M-BH2.1-apr-d-07-0-UI s1 Mus musculus cDNA, 3 end/clone = UI-M-BH2 1-apr-d-07-0-UI/ | 364.7 | P | 357.7 | P | 347.8 | P |
| | | clone_end = 3/gb = AW124128/gi = 6099658/ug = Mm 46624/len = 357/STRA = for | | | | | | |
| 7 | 100126_at | Cluster Incl AA967263 vz70e04 r1 Mus musculus cDNA, 5 end/clone = IMAGE-1331838/clone_end = 5/gb = AA967263/ | 572.1 | P | 514.3 | P | 667.4 | P |
| | | gi = 3141156/ug = Mm 4748/len = 432/STRA = for | | | | | | |
| 7 | 100554_at | Cluster Incl AF053367 Mus musculus carboxyl terminal LIM domain protein (Ldb3) mRNA, complete cds/cds = (60,1040)/ | 609.8 | P | 336.3 | P | 396.3 | P |
| | | gb = AF053367/gi = 2996195/ug = Mm 5567/len = 1256/STRA = for | | | | | | |
| 7 | 100555_at | Cluster Incl AI846152.UI-M-AN1-afj-h-05-0-UI.s2 Mus musculus cDNA, 3 end/clone = UI-M-AN1-afj-h-05-0-UI/ | 931.9 | P | 789.8 | P | 653.3 | P |
| | | clone_end = 3/gb = AI846152/gi = 5490058/ug = Mm 56/len = 408/STRA = for | | | | | | |
| 7 | 100557_g_at | Cluster Incl AW121930-UI-M-BH2.3-aoj-d-12-0-UI s1 Mus musculus cDNA, 3 end/clone = UI-M-BH2 3-aoj-d-12-0-UI/ | 1368.5 | P | 1154.3 | P | 1173.9 | P |
| | | clone_end = 3/gb = AW121930/gi = 6097374/ug = Mm 5612/len = 467/STRA = for | | | | | | |
| 7 | 100579_s_at | Cluster Incl U91848 Mus musculus clathrin light chain 2 mRNA, complete cds/cds = (108,815)/gb = U91848/gi = 1916987/ | 1125.6 | P | 856 | P | 1053.6 | P |
| | | ug = Mm 6072/len = 1089/STRA = for | | | | | | |
| 7 | 100613_at | Cluster Incl AF030635: Mus musculus FK-506 binding protein homolog (SAM11) mRNA, complete cds/cds = (28,1095)/ | 1098.2 | P | 664.5 | P | 888 | P |
| | | gb = AF030635/gi = 2623223/ug = Mm 6565/len = 1514/STRA = for | | | | | | |
| 7 | 100622_at | Cluster Incl AF093857: untitled/cds = (49,723)/gb = AF093857/gi = 4139185/ug = Mm 6587/len = 1436/STRA = for | 941.4 | P | 692.4 | P | 856.9 | P |
| 7 | 101078_at | Cluster Incl Y16258 Basigin/cds = (0,773)/gb = Y18258/gi = 2808471/ug = Mm 726/len = 1244/STRA = for | 2452.1 | P | 2118.7 | P | 2309.2 | P |
| 7 | 101086_f_at | Cluster Incl X63866 Mus musculus mRNA for cellular nucleic acid binding protein/cds = (104,616)/gb = X63866/gi = 50470/ | 1982.6 | P | 1575.6 | P | 1578.5 | P |
| | | ug = Mm 7335/len = 1492/STRA = for | | | | | | |
| 7 | 101088_f_at | Cluster Incl U20326 Mus musculus cellular nucleic acid binding protein mRNA, complete cds/cds = (119,655)/gb = U20326/ | 1971.1 | P | 1821.2 | P | 1700 | P |
| | | gi = 854674/ug = Mm 7335/len = 1519/STRA = for | | | | | | |
| 7 | 101095_at | Cluster Inc L23769 Microfibril-associated glycoprotein/cds = (80,631)/gb = L23769/gi = 440831/ug = Mm 7386/len = 1020/ | 1885.9 | P | 498.6 | P | 629.4 | P |
| | | STRA = for | | | | | | |
| 7 | 101107_at | Cluster Incl U81829 Calumenin/cds = (35,982)/gb = U81829/gi = 2323276/ug = Mm 7515/len = 3147/STRA = for | 1571.7 | P | 1034.9 | P | 1277.3 | P |
| 7 | 101110_at | Cluster Incl AF064749 Mus musculus type VI collagen alpha 3 subunit mRNA, complele cds/cds = (61,8034)/gb = AF064749/ | 5137.3 | P | 2190.4 | P | 2989.7 | P |
| | | gi = 3236369/ug = Mm 7562/len = 8775/STRA = for | | | | | | |
| 7 | 101499_at | Cluster Incl U94479 Mus musculus Integrin binding protein kinase mRNA, complete cds/cds = (113,1471)/gb = U94479/ | 1181.8 | P | 670 | P | 971.7 | P |
| | | gi = 2739449/ug = Mm 8131/len = 1750/STRA = for | | | | | | |
| 7 | 101514_at | Cluster Incl AF041433 Mus musculus bet3 (Bet3) mRNA, complete cds/cds = (82,624)/gb = AF041433/gi = 2791805/ | 515.4 | P | 358.6 | P | 532.6 | P |
| | | ug = Mm 8392/len = 753/STRA = for | | | | | | |
| 7 | 101991_at | Cluster Incl D16215 Mouse mRNA for flavin-containing monooxygenase, complete cds/cds = (206,1804)/gb = D16215/ | 273.2 | P | 187.7 | P | 240.8 | P |
| | | gi = 286077/ug = Mm 976/len = 2126/STRA = rev | | | | | | |
| 7 | 102044_at | Cluster Incl AF100777 Mus musculus connective tissue growth factor related protein WISP-1 (Wisp1) mRNA, complete cds/ | 273.8 | P | 211.3 | P | 190.7 | P |
| | | cds = (78,1181)/gb = AF100777/gi = 4028576/ug = Mm 10222/len = 1766/STRA = for | | | | | | |
| 7 | 102322_at | Cluster Incl AF061017 UDP-glucose dehydrogenase/cds = (110,1591)/gb = AF061017/gi = 3127128/ug = Mm 10709/ | 1295.4 | P | 1489.9 | P | 1391.3 | P |
| | | len = 2328/STRA = for | | | | | | |
| 7 | 102395_at | Cluster Incl Z38110 M musculus PMP22 mRNA for peripheral myelin protein/cds = (0,482)/gb = Z38110/gi = 558353/ | 1422.6 | P | 779.1 | P | 803.1 | P |
| | | ug = Mm 1237/len = 483/STRA = for | | | | | | |
| 7 | 102752_at | Cluster Incl AF072697 Mus musculus SHYC (Shyc) mRNA, complete cds/cds = (33,3794)/gb = AF072697/gi = 3293550/ | 1015 | P | 890 | P | 902.6 | P |
| | | ug = Mm.12912/len = 4112/STRA = for | | | | | | |
| 7 | 102772_at | Cluster Incl J02995 Abelson murine leukemia oncogene/cds = (3565,4056)/gb = J02995/gi = 191566/ug = Mm 1318/ | 254.4 | P | 184.2 | P | 241.1 | P |
| | | len = 5058/STRA = for | | | | | | |
| 7 | 102811_at | Cluster Incl X96639 Exostoses (multiple) 1/cds = (594,2831)/gb = X96639/gi = 1235558/ug = Mm 1424/len = 2965/STRA = for | 642.3 | P | 643.8 | P | 564.5 | P |
| 7 | 102983_at | Cluster Incl U58992.MAD homolog 1 (Drosophila)/cds = (70,1467)/gb = U58992/gi = 1518644/ug = Mm 15185/len = 1639/ | 411.1 | P | 382.5 | P | 354.7 | P |
| | | STRA = for | | | | | | |
| 7 | 102984_g_at | Cluster Incl U58992: MAD homolog 1 (Drosophila)/cds = (70,1467)/gb = U58992/gi = 1518644/ug = Mm 15185/len = 1639/ | 1026.7 | P | 916.9 | P | 867.5 | P |
| | | STRA = for | | | | | | |
| 7 | 103054_at | Cluster Incl U37500: Mus musculus RNA polymerase II largest subunit (RPB1) mRNA, complete cds/cds = (69,5969)/ | 640.3 | P | 593.2 | P | 665.1 | P |
| | | gb = U37500/gi = 2145090/ug = Mm.16533/len = 8270/STRA = rev | | | | | | |
| 7 | 103100_at | Cluster Incl AI788543 uk46c09 x1 Mus musculus cDNA, 3 end/clone = IMAGE-1972048/clone_end = 3/gb = AI788543/ | 648.9 | P | 544.7 | P | 527.1 | P |
| | | gi-5336259/ug = Mm 17569/len = 656/STRA = for | | | | | | |
| 7 | 103353_f_at | Cluster Incl D50834 Cytochrome P450. subfamily IV B, polypeptide 1/cds = (18,1553)/gb = D50834/gi = 854665/ | 530.4 | P | 449.4 | P | 501.7 | P |
| | | ug = Mm 1840/len = 1871/STRA = for | | | | | | |
| 7 | 103379_at | Cluster Incl AW046579 UI-M-BH1-alb-e-03-0-UI s1 Mus musculus cDNA, 3 end/clone = UI-M-BH1-alb-e-03-0-UI/ | 131.5 | P | 88.1 | P | 126.9 | P |
| | | clone_end = 3/gb = AW046579/gi = 5907108/ug = Mm 18616/len = 301/STRA = rev | | | | | | |
| 7 | 103608_at | Cluster Incl AI842638.UI-M-AQ1-aed-f-04-0-UI s1 Mus musculus cDNA, 3 end/clone = UI-M-AQ1-aed-f-04-0-UI/ | 321.3 | P | 257.1 | P | 291.2 | P |
| | | clone_end = 3/gb = AI842636/gi = 5476849/ug = Mm 20175/len = 311/STRA = for | | | | | | |
| 7 | 104100_at | Cluster Incl AI845915: UI-M-AK1-aex-d-02-0-UI.s1 Mus musculus cDNA, 3 end/clone = UI-M-AK1-aex-d-02-0-UI/ | 2697.1 | P | 1687.8 | P | 2435.1 | P |
| | | clone_end = 3/gb = AI845915/gi = 5489821/ug = Mm.21864/len = 208/STRA = for | | | | | | |
| 7 | 104343_f_at | Cluster Incl AI845798-UI-M-AQ1-aeb-h-07-0-UI s1 Mus musculus cDNA, 3 end/clone = UI-M-AQ1-aeb-h-07-0-UI/ | 405.1 | P | 398.8 | P | 290.2 | P |
| | | clone_end = 3/gb = AI845798/gi = 5489704/ug = Mm 22526/len = 184/STRA = for | | | | | | |
| 7 | 104587_at | Cluster Incl U69176: Laminin, alpha 4/cds = (386,5833)/gb = U69176/gi = 1568624/ug = Mm 2399/len = 5993/STRA = for | 683.4 | P | 351.9 | P | 562.1 | P |
| 7 | 104712_at | Cluster Incl L00039 Myelocytomalosis oncogene/cds = (15,1334)/gb = L00039/gi = 192621/ug = Mm 2444/len = 1707/ | 275.2 | P | 111.3 | P | 122 | P |
| | | STRA = for | | | | | | |
| 7 | 104756_at | Cluster Incl AA739415: vv53c01 r1 Mus musculus cDNA, 5 end/clone = IMAGE-1226112/clone_end = 5/gb = AA739415/ | 440.6 | P | 352 | P | 377.9 | P |
| | | gi = 2775601/ug = Mm.24788/len = 451/STRA = for | | | | | | |
| 7 | 94394_at | Cluster Incl M21019: Harvey rat sarcoma oncogene, subgroup R/cds = (11,667)/gb = M21019/gi = 200676/ug = Mm 257/ | 306.3 | P | 226.7 | P | 202 | P |
| | | len = 949/STRA = rev | | | | | | |
| 7 | 94415_at | Cluster Incl AA710439 vt42e01 r1 Mus musculus cDNA, 5 end/clone = IMAGE-1165752/clone_end = 5/gb = AA710439/ | 508.3 | P | 347.6 | P | 440.8 | P |
| | | gi = 2720357/ug = Mm 26147/len = 510/STRA = for | | | | | | |
| 7 | 94942_at | Cluster Incl AI854108: UI-M-BH0-ajw-c-12-0-UI s1 Mus musculus cDNA, 3 end/clone = UI-M-BH0-ajw-c-12-0-UI/ | 404 | P | 422.1 | P | 249.1 | P |
| | | clone_end = 3/gb = AI854108/gi = 5498014/ug = Mm 26944/len = 581/STRA = rev | | | | | | |
| 7 | 95609_at | Cluster Incl AA869927: vq19d11.r1 Mus musculus cDNA, 5 end/clone = IMAGE-1094709/clone_end = 5/gb = AA869927/ | 385.6 | P | 338 | P | 447.6 | P |
| | | gi = 2965372/ug = Mm 27987/len = 497/STRA = for | | | | | | |
| 7 | 96178_at | Cluster Incl AI850636 UI-M-BG1-aik-e-04-0-UI.s1 Mus musculus cDNA, 3 end/clone = UI-M-BG1-aik-e-04-0-UI/ | 426.4 | P | 387.2 | P | 325.4 | P |
| | | clone_end = 3/gb = AI850636/gi = 5494542/ug = Mm 28426/len = 391/STRA = for | | | | | | |
| 7 | 96733_at | Cluster Incl AW124597 UI-M-BH2 1-apt-h-01-0-UI s1 Mus musculus cDNA, 3 end/clone = UI-M-BH2 1-apt-h-01-0-UI/ | 546.4 | P | 508.2 | P | 581 | P |
| | | clone_end = 3/gb = AW124597/gi = 6100132/ug = Mm 28893/len = 499/STRA = for | | | | | | |
| 7 | 96767_at | Cluster Incl AF098633 Mus musculus GLUT4 vesicle protein mRNA, partial cds/cds = (0,791)/gb = AF098633/gi = 4200441/ | 304 | P | 120.1 | P | 115.8 | P |
| | | ug = Mm 29010/len = 1192/STRA = rev | | | | | | |
| 7 | 97359_at | Cluster Incl AI851160 UI-M-BH0-akg-f-01-0-UI.s1 Mus musculus cDNA, 3 end/clone = UI-M-BH0-akg-f-01-0-UI/ | 444.7 | P | 308.9 | P | 304.7 | P |
| | | clone_end = 3/gb = AI851160/gi = 5495031/ug = Mm 29649/len = 447/STRA = for | | | | | | |
| 7 | 98474_r_at | Cluster Incl U83903: Tumor necrosis factor induced protein 6/cds = (63,890)/gb = U83903/gi = 2062474/ug = Mm 3509/ | 323.7 | P | 106.8 | P | 213.1 | P |
| | | len = 1605/STRA = for | | | | | | |
| 7 | 98475_at | Cluster Incl U69262: Mus musculus matrilin-2 precursor mRNA, complete cds/cds = (250,3120)/gb = U69262/gi = 2072791/ | 253.4 | P | 208.9 | P | 178.3 | P |
| | | ug = Mm 3511/len = 3571/STRA = for | | | | | | |
| 7 | 98508_s_at | Cluster Incl D84376: Mus musculus mRNA for Phosphatidic acid phosphatase, complete cds/cds = (286,1137)/gb = D84376/ | 377. 9 | P | 194.7 | P | 399.2 | P |
| | | gi = 1487872/ug = Mm 35836/len = 1212/STRA = for | | | | | | |
| 7 | 99031_at | Cluster Incl AI854005 UI-M-BH0-arv-h-09-0-UI.s1 Mus musculus cDNA, 3 end/clone = UI-M-BH0-arv-h-09-0-UI/ | 335.7 | P | 369.3 | P | 350.8 | P |
| | | clone_end = 3/gb = AI854005/gi = 5497911/ug = Mm.38930/len = 538/STRA = for | | | | | | |
| 7 | 99038_at | Cluster Incl L24554 Mus musculus adenylosuccinate synthetase mRNA, complete cds/cds = (82,1452)/gb = L24554/ | 494.4 | P | 427.2 | P | 434.3 | P |
| | | gi = 404058/ug = Mm 39061/len = 1559/STRA = for | | | | | | |
| 7 | 99039_g_at | Cluster Incl L24554 Mus musculus adenylosuccinate synthetase mRNA, complete cds/cds = (82,1452)/gb = L24554/ | 412.8 | P | 378.3 | P | 369.5 | P |
| | | gi = 404056/ug = Mm 39061/len = 1559/STRA = for | | | | | | |
| 7 | 99535_at | Cluster Incl AW047630 UI-M-BH1-akm-g-06-0-UI s1 Mus musculus cDNA, 3 end/clone = UI-M-BH1-akm-g-06-0-UI/ | 688.5 | P | 571 | P | 488.8 | P |
| | | clone_end = 3/gb = AW047630/gi = 5908159/ug = Mm 42187/len = 385/STRA = for | | | | | | |
| 7 | 100442_at | Cluster Incl AA656541 vs22a03 r1 Mus musculus cDNA, 5 end/clone = IMAGE-1138924/clone_end = 5/gb = AA656541/ | 313.4 | P | 231.3 | P | 266.2 | P |
| | | gi = 2592695/ug = Mm 4810/len = 305/STRA = for | | | | | | |
| 7 | 100466_f_at | Cluster Incl AA655823: vs48b07 r1 Mus musculus cDNA, 5 end/clone = IMAGE-1149493/clone_end = 5/gb = AA655823/ | 2039.5 | P | 1099.3 | P | 1182.2 | P |
| | | gi = 2591977/ug = Mm.4923/len = 356/STRA = for | | | | | | |
| 7 | 100974_at | Cluster Incl AI844250 UI-M-AL1-ahk-g-10-0-UI s1 Mus musculus cDNA, 3 end/clone = UI-M-AL1-ahk-g-10-0-UI/ | 492.3 | P | 282.1 | P | 443 | P |
| | | clone_end = 3/gb = AI844250/gi = 5488156/ug = Mm.6667/len = 237/STRA = rev | | | | | | |
| 7 | 101921_at | Cluster Incl D86563: RAB4A, member RAS oncogene family/cds = (129,770)/gb = D86583/gi = 2706877/ug = Mm 9221/ | 191.5 | P | 224.3 | P | 213.2 | P |
| | | len = 1385/STRA = for | | | | | | |
| 7 | 103310_at | Cluster Incl AA220427 mv70b05 r1 Mus musculus cDNA, 5 end/clone = IMAGE-660369/clone_end = 5/gb = AA220427/ | 127.6 | P | 150.1 | P | 162 | P |
| | | gi = 1838880/ug = Mm 21184/len = 261/STRA = for | | | | | | |
| 7 | 103549_at | Cluster Incl AW061260: UI-M-BH1-amj-f-05-0-UI s2 Mus musculus cDNA, 3 end/clone = UI-M-BH1-amj-f-05-0-UI/ | 785.7 | P | 560.3 | P | 629.9 | P |
| | | clone_end = 3/gb = AW061260/gi = 6009011/ug = Mm 23742/len = 185/STRA = for | | | | | | |
| 7 | 103555_at | Cluster Incl AI843520 UI-M-AO1-aeh-c-10-0-UI s1 Mus musculus cDNA, 3 end/clone = UI-M-AO1-aeh-c-10-0-UI/ | 2937.7 | P | 2703 | P | 2748.5 | P |
| | | clone_end = 3/gb = AI843520/gi = 5477733/ug = Mm 23842/len = 387/STRA = for | | | | | | |
| 7 | 104235_at | Cluster Incl AW122882 UI-M-BH2.1-apa-c-06-0-UI s1 Mus musculus cDNA, 3 end/clone = UI-M-BH2 1-apa-c-06-0-UI/ | 1133.8 | P | 972 | P | 892.8 | P |
| | | clone_end = 3/gb = AW122882/gi = 6098412/ug = Mm 28117/len = 316/STRA = for | | | | | | |
| 7 | 104410_at | Cluster Incl AW124785: UI-M-BH2.1-aqa-h-06-0-UI s1 Mus musculus cDNA, 3 end/clone = UI-M-BH2 1-aqa-h-06-0-UI/ | 1474.7 | P | 866.5 | P | 943.3 | P |
| | | clone_end = 3/gb = AW124785/gi = 6100315/ug = Mm 2883/len = 417/STRA = for | | | | | | |
| 7 | 104464_a_at | Cluster Incl AI642389: vs90e02 x1 Mus musculus cDNA, 3 end/clone = IMAGE-1153562/clone_end = 3/gb = AI642389/ | 567.2 | P | 249.3 | P | 270.7 | P |
| | | gi = 4720864/ug = Mm 29644/len = 309/STRA = for | | | | | | |
| 7 | 104494_at | Cluster Incl AI642098: ub75g10.x1 Mus musculus cDNA, 3 end/clone = IMAGE-1383618/clone_end = 3/gb = AI642098/ | 507.7 | P | 625.1 | P | 515 | P |
| | | gi = 4720573/ug = Mm 3038/len = 448/STRA = for | | | | | | |
| 7 | 92925_at | Cluster Incl M61007 CCAAT/enhancer binding protein (C/EBP), beta/cds = (16,906)/gb = M61007/gi = 191775/ug = Mm 4883/ | 2552.2 | P | 1814.6 | P | 1770.2 | P |
| | | len = 1416/STRA = rev | | | | | | |
| 7 | 93440_at | Cluster Incl AA692530 vt59h11.r1 Mus musculus cDNA, 5 end/clone = IMAGE-1167429/clone_end = 5/gb = AA692530/ | 354.1 | P | 313.2 | P | 252 | P |
| | | gi = 2693468/ug = Mm 6636/len = 414/STRA = for | | | | | | |
| 7 | 93482_at | Cluster Incl AI117835 uc41b01 r1 Mus musculus cDNA, 5 end/clone = IMAGE-1400521/clone_end = 5/gb = AI117835/ | 594.5 | P | 643.8 | P | 533.4 | P |
| | | gi = 3518159/ug = Mm 7128/len = 397/STRA = rev | | | | | | |
| 7 | 93924_f_at | Cluster Incl M13443 Mouse alpha-tubulin isotype M-alpha-7 mRNA, complete cds/cds = (39,1391)/gb = M13443/gi = 202218/ | 1086.2 | P | 653.9 | P | 737.4 | P |
| | | ug = Mm 89866/len = 1424/STRA = for | | | | | | |
| 7 | 97740_at | Cluster Incl AI642662 vw01d07.x1 Mus musculus cDNA, 3 end/clone = IMAGE-1230637/clone_end = 3/gb = AI642662/ | 234.4 | P | 153 | P | 165.9 | P |
| | | gi = 4721137/ug = Mm 3994/len = 307/STRA = for | | | | | | |
| 7 | 100708_at | Cluster Incl X13605: Murine mRNA for replacement variant histone H3.3/cds = (73,483)/gb = X13605/gi = 51197/ | 4998.8 | P | 4868.9 | P | 3137.8 | P |
| | | ug = Mm.18516/len = 1619/STRA = for | | | | | | |
| 7 | 93842_at | Cluster Incl AI196645: ui53d07 y1 Mus musculus cDNA, 5 end/clone = IMAGE-1886125/clone_end = 5/gb = AI196645/ | 297.8 | P | 32.2 | M | 264.3 | P |
| | | gi = 3749251/ug = Mm 21622/len = 1009/STRA = for | | | | | | |
| 7 | 93974_at | Cluster Incl AW212475 uo89c05.x1 Mus musculus cDNA, 3 end/clone = IMAGE-2649704/clone_end = 3/gb = AW212475/ | 700.1 | P | 328.5 | P | 405.5 | P |
| | | gi = 6518562/ug = Mm 21679/len = 230/STRA = for | | | | | | |
| 7 | 93996_at | Cluster Incl X01026: Mouse mRNA fragment for repetitive element B2/cds = (0,119)/gb = X01026/gi = 53957/ug = Mm 21758/ | 279.9 | A | 227.4 | P | 254 | P |
| | | len = 354/STRA = for | | | | | | |
| 7 | 95469_at | Cluster Incl AA734444 vr90g04.r1 Mus musculus cDNA, 5 end/clone = IMAGE-1136022/clone_end = 5/gb = AA734444/ | 202.8 | P | 164 | P | 173.9 | P |
| | | gi = 2756111/ug = Mm 24786/len = 252/STRA = for | | | | | | |
| 7 | 96637_at | Cluster Incl U33005 Mus musculus tbc1 mRNA, complete cds/cds = (402,3827)/gb = U33005/gi = 988220/ug = Mm 27271/ | 433.1 | P | 222.6 | P | 303.6 | P |
| | | len = 4039/STRA = for | | | | | | |
| 7 | 96703_at | Cluster Incl AB029448: Mus musculus mRNA for Dixin-1, complete cds/cds = (212,2539)/gb = AB029448/gi = 6520236/ | 1126.1 | P | 494.7 | M | 578.4 | P |
| | | ug = Mm.27578/len = 2764/STRA = for | | | | | | |
| 7 | 96713_at | Cluster Incl AF052453 3-phosphoadenosine 5-phosphosulfate synthase 2/cds = (130,1995)/gb = AF052453/gi = 3342265/ | 187.2 | P | 106.7 | P | 177 | P |
| | | ug = Mm 27646/len = 1996/STRA = for | | | | | | |
| 7 | 98942_r_at | Cluster Incl AW125284: UI-M-BH2 1-apy-a-11-0-UI s1 Mus musculus cDNA, 3 end/clone = UI-M-BH2 1-apy-a-11-0-UI/ | 237.9 | P | 132.4 | P | 73.5 | P |
| | | clone_end = 3/gb = AW125284/gi = 6100814/ug = Mm.28697/len = 336/STRA = for | | | | | | |
| 7 | 96283_at | Cluster Incl AI849180 UI-M-AJ1-agy-f-10-0-UI s1 Mus musculus cDNA, 3 end/clone = UI-M-AJ1-agy-f-10-0-UI/ | 761.2 | P | 395.5 | P | 864.7 | P |
| | | clone_end = 3/gb = AI849180/gi = 5493086/ug = Mm.29870/len = 397/STRA = for | | | | | | |
| 7 | 96930_at | Cluster Incl AI844128 UI-M-AL1-ahj-d-06-0-UI.s1 Mus musculus cDNA, 3 end/clone = UI-M-AL1-ahj-d-06-0-UI/ | 232.6 | P | 156 | A | 249.5 | P |
| | | clone_end = 3/gb = AI844128/gi = 5488034/ug = Mm.30169/len = 328/STRA = for | | | | | | |
| 7 | 97444_at | Cluster Incl AI844520 UI-M-AJ1-ahf-a-08-0-UI s2 Mus musculus cDNA, 3 end/clone = UI-M-AJ1-ahf-a-08-0-UI/ | 275.6 | P | 175.3 | A | 163.7 | P |
| | | clone_end = 3/gb = AI844520/gi = 5488426/ug = Mm.30241/len = 491/STRA = for | | | | | | |
| 7 | 98626_at | Cluster Incl AW047503 UI-M-BH1-ama-b-05-0-UI s1 Mus musculus cDNA, 3 end/clone = UI-M-BH1-ama-b-05-0-UI/ | 274.4 | P | 145.9 | P | 282.1 | P |
| | | clone_end = 3/gb = AW047503/gi = 5908032/ug = Mm.38746/len = 264/STRA = for | | | | | | |
| 7 | 100592_at | Cluster Incl AI929971 uI60a06 y1 Mus musculus cDNA, 5 end/clone = IMAGE-2123410/clone_end = 5/gb = AI929971/ | 1174.5 | P | 1039.2 | P | 855.4 | P |
| | | gi = 5666012/ug = Mm 63/len = 510/STRA = rev | | | | | | |
| 7 | 102378_at | Cluster Incl U02487: Kras oncogene-associated gene/cds = (27,677)/gb = U02487/gi = 437199/ug = Mm.1209/len = 4420/ | 313.8 | P | 115.2 | P | 49.9 | A |
| | | STRA = for | | | | | | |
| 7 | 102387_at | Cluster Incl AA799042 vn40d08 r1 Mus musculus cDNA, 5 end/clone = IMAGE-1023663/clone_end = 5/gb = AA799042/ | 509.7 | P | 462.1 | P | 592.9 | P |
| | | gi = 2861997/ug = Mm.12183/len = 350/STRA = for | | | | | | |
| 7 | 103032_at | Cluster Incl AF038008: Mus musculus tyrosylprotein sulfotransferase-1 mRNA, complete cds/cds = (210,1322)/ | 242 | P | 172.5 | P | 148.2 | A |
| | | gb = AF038008/gi = 3046915/ug = Mm.16084/len = 1867/STRA = for | | | | | | |
| 7 | 104389_at | Cluster Incl AW049360 UI-M-BH1-ane-a-10-0-UI s1 Mus musculus cDNA, 3 end/clone = UI-M-BH1-ane-a-10-0-UI/ | 204.6 | P | 185.3 | M | 324.7 | P |
| | | clone_end = 3/gb = AW049360/gi = 5909889/ug = Mm.22712/len = 358/STRA = for | | | | | | |
| 7 | 94384_at | Cluster Incl X67644 M musculus gly96 mRNA/cds = UNKNOWN/gb = X67644/gi = 287803/ug = Mm 25613/len = 1938/ | 1268 | P | 447.4 | P | 946.2 | P |
| | | STRA = for | | | | | | |
| 7 | 94948_at | Cluster Incl AF097511: Mus musculus zyxin related protein-1 (Zrp1) mRNA, complete cds/cds = (66,1508)/gb = AF097511/ | 493.3 | P | 257.4 | P | 374.1 | P |
| | | gi = 3851623/ug = Mm.27063/len = 1664/STRA = for | | | | | | |
| 7 | 95037_at | Cluster Incl AW046639 UI-M-BH1-ald-d-10-0-UI s1 Mus musculus cDNA, 3 end/clone = UI-M-BH1-ald-d-10-0-UI/ | 354.8 | A | 189.2 | P | 405.4 | P |
| | | clone_end = 3/gb = AW046639/gi = 5907168/ug = Mm.27557/len = 338/STRA = for | | | | | | |
| 7 | 96752_at | Cluster Incl M90551 Intercellular adhesion molecule/cds = (75,1688)/gb = M90551/gi = 459870/ug = Mm 28973/len = 2540/ | 361.5 | P | 170.6 | P | 270.4 | P |
| | | STRA = for | | | | | | |
| 7 | 97404_at | Cluster Incl AW048244 UI-M-BH1-aly-b-04-0-UI.s1 Mus musculus cDNA, 3 end/clone = UI-M-BH1-aly-b-04-0-UI/ | 234.1 | P | 213.9 | P | 242.8 | P |
| | | clone_end = 3/gb = AW048244/gi = 5908773/ug = Mm 29901/len = 328/STRA = for | | | | | | |
| 7 | 98988_at | Cluster Incl AA614971 Vo32e09 r1 Mus musculus cDNA, 5 end/clone = IMAGE-1051624/clone_end = 5/gb = AA614971/ | 1158.4 | P | 171 | P | 118.6 | P |
| | | gi = 2502199/ug = Mm 3732/len = 137/STRA = for | | | | | | |
| 7 | 100977_at | Cluster Incl AA691492 vu15h09 r1 Mus musculus cDNA, 5 end/clone = IMAGE-1180769/clone_end = 5/gb = AA691492/ | 272.8 | P | 286.2 | P | 216.8 | P |
| | | gi = 2692430/ug = Mm 6691/len = 449/STRA = for | | | | | | |
| 7 | 101834_at | Cluster Incl Z14249 Protein kinase, mitogen activated kinase 3/cds = UNKNOWN/gb = Z14249/gi = 50858/ug = Mm 8385/ | 495.5 | P | 498.6 | P | 617.5 | M |
| | | len = 1718/STRA = for | | | | | | |
| 7 | 103311_at | Cluster Incl AW121299 UI-M-BH2.3-aog-f-02-0-UI.s1 Mus musculus cDNA, 3 end/clone = UI-M-BH2.3-aog-f-02-0-UI/ | 509.3 | P | 273.8 | A | 748.6 | P |
| | | clone_end = 3/gb = AW121299/gi = 6096632/ug = Mm 21199/len = 236/STRA = for | | | | | | |
| 7 | 104019_at | Cluster Incl AI851068 UI-M-BH0-ajv-h-07-UI s1 Mus musculus cDNA, 3 end/clone = UI-M-BH0-ajv-h-07-0-UI/ | 145 | P | 56.6 | P | 83.4 | P |
| | | clone end = 3/gb = AI851068/gi = 5494974/ug = Mm 26936/len = 441/STRA = for | | | | | | |
| 7 | 100277_at | Cluster Incl X69619 lnhibin beta-A/cds = (172,1446)/gb = X69619/gi = 50145/ug = Mm 8042/len = 1529/STRA = for | 393.8 | P | 309.7 | P | 248.3 | P |
| 7 | 93062_at | Cluster Incl AW045881 UI-M-BH1-aky-h-01-0-UI.s1 Mus musculus cDNA, 3 end/clone = UI-M-BH1-aky-h-01-0-UI/ | 685.6 | P | 448.2 | P | 351 | A |
| | | clone end = 3/gb = AW045881/gi = 5906410/ug = Mm.1555/len = 561/STRA = for | | | | | | |
| 7 | 93550_at | Cluster Incl D88792: Mouse mRNA for double LIM protein-1, complete cds/cds = (6,587)/gb = D88792/gi = 1695701/ | 412 | P | 83.1 | M | 122.2 | A |
| | | ug = Mm 2020/len = 822/STRA = for | | | | | | |
| 7 | 96035_at | Cluster Incl L47335 Mus musculus branched chain alpha ketoacid decarboxylase E1a subunit mRNA, complete cds/ | 201.8 | P | 208.6 | P | 140.3 | P |
| | | cds = (17,1345)/gb = L47335/gi = 992564/ug = Mm 25848/len = 1681/STRA = for | | | | | | |
| 7 | 95669_g_at | Cluster Incl AI840972 UI-M-AK0-adk-e-04-0-UI.s1 Mus musculus cDNA, 3 end/clone = UI-M-AK0-adk-e-04-0-UI/ | 514.3 | P | 235.1 | P | 353.7 | P |
| | | clone_end = 3/gb = AI840972/gi = 5475185/ug = Mm.29580/len = 456/STRA = for | | | | | | |
| 7 | 96885_at | Cluster Incl AW122271.UI-M-BH2 2-aov-h-12-0-UI s1 Mus musculus cDNA, 3 end/clone = UI-M-BH2 2-aov-h-12-0-UI/ | 770.7 | P | 376.4 | A | 492.8 | P |
| | | clone_end = 3/gb = AW122271/gi = 6097746/ug = Mm 30083/len = 240/STRA = for | | | | | | |
| 7 | 99167_at | Cluster Incl AW122550 UI-M-BH2.2-aox-d-08-0-UI.s1 Mus musculus cDNA, 3 end/clone = UI-M-BH2 2-aox-d-08-0-UI/ | 218.8 | P | 145.1 | A | 149.2 | P |
| | | clone_end = 3/gb = AW122550/gi_6098074/ug = Mm 41687/len = 462/STRA = for | | | | | | |
| 7 | 103037_at | Cluster Incl U8366 Mus musculus cardiotrophin-1 mRNA complete cds/cds = (19,630)/gb = 18366)/gi = 710331 | 56.8 | P | 20.12 | P | 163.3 | P |
| | | ug = Mm 1616/len = 1345/stra = STRA = for | | | | | | |
| 7 | 103044_g_at | Cluster Incl Z35294: M. musculus MTCP-1 gene/cds = (540,746)/gb = Z35294/gi = 603859/ug = Mm.16366/len = 954/ | 636.6 | P | 367.4 | A | 318 | P |
| | | STRA = for | | | | | | |
| 7 | 103579_at | Cluster Incl X53247 M musculus EN-7 mRNA/cds = (36,614)/gb = X53247/gi = 50836/ug = Mm 1972/len = 1255/STRA = for | 900.5 | M | 651.5 | P | 763.9 | A |
| 7 | 104306_at | Cluster Incl AI847886 UI-M-AM1-afu-g-07-0-UI.s1 Mus musculus cDNA, 3 end/clone = UI-M-AM1-afu-g-07-0-UI/ | 374.3 | P | 264.5 | A | 386.1 | P |
| | | clone_end = 3/gb = AI847886/gi = 5491792/ug = Mm 22369/len = 392/STRA = for | | | | | | |
| 7 | 94332_at | Cluster Incl AI882555 ub97f03 r1 Mus musculus cDNA, 5 end/clone = IMAGE-1396445/clone_end = 5/gb = AI882555/ | 133.2 | P | 87.3 | P | 69.2 | P |
| | | gi = 5567644/ug = Mm 2490/len = 329/STRA = for | | | | | | |
| 7 | 94377_at | Cluster Incl AI854793: UI-M-BH0-aka-f-02-0-UI s1 Mus musculus cDNA, 3 end/clone = UI-M-BH0-aka-f-02-0-UI/ | 34.4 | P | 7 | A | 51.5 | P |
| | | clone_end = 3/gb = AI854793/gi = 5498699/ug = Mm 25547/len = 359/STRA = for | | | | | | |
| 7 | 97353_at | Cluster Incl AI837497: UI-M-AL0-abr-g-08-0-UI s1 Mus musculus cDNA, 3 end/clone = UI-M-AL0-abr-g-08-0-UI/ | 113 | P | 35.2 | P | 180.3 | P |
| | | clone_end = 3/gb = AI837497/gi = 5471710/ug = Mm 29629/len = 224/STRA = for | | | | | | |
| 7 | 98492_at | Cluster Incl AA920419 vz26a09 r1 Mus musculus cDNA, 5 end/clone = IMAGE-1327576/clone_end = 5/gb = AA920419/ | 938.6 | P | 740.4 | A | 695.7 | P |
| | | gi = 3067198/ug = Mm 35600/len = 371/STRA = for | | | | | | |
| 7 | 99015_at | Cluster Incl U33626 Promyelocytic leukemia/cds = (86,2512)/gb = U33626/gi = 995828/ug = Mm 38497/len = 3760/STRA = for | 384.6 | P | 211.9 | P | 240 | P |
| 7 | 99444_at | Cluster Incl AJ250490.Mus musculus mRNA for receptor activity modifying protein 2 (Ramp2 gene)/cds = (30,599)/ | 318.5 | P | 223.6 | P | 232.7 | P |
| | | gb = AJ250490/gi = 6119626/ug = Mm 40358/len = 808/STRA = for | | | | | | |
| 7 | 102736_at | Cluster Incl M19681 Small inducible cytokine A2/cds = (82,528)/gb = M19681/gi = 193486/ug = Mm.145/len = 798/STRA = for | 290.4 | P | 7.3 | P | 62.6 | P |
| 7 | 103690_at | Cluster Incl AW125574 UI-M-BH2.2-aqj-c-05-0-UI.s1 Mus musculus cDNA, 3 end/clone = UI-M-BH2.2-aqj-c-05-0-UI/ | 152 | P | 203.4 | P | 94.8 | P |
| | | clone_end = 3/gb = AW125574/gi = 6101104/ug = Mm 23955/len = 566/STRA = rev | | | | | | |
| 7 | 93376_at | Cluster Incl AA673486 vp49f04 r1 Mus musculus cDNA, 3 end/clone = IMAGE-1080031/clone_end = 3/gb = AA673486/ | 201.9 | P | 125.2 | P | 100.4 | A |
| | | gi = 2646768/ug = Mm 6156/len = 477/STRA = rev | | | | | | |
| 7 | 102114_f_at | Cluster Incl AI326963 mj76a06.x1 Mus musculus cDNA, 3 end/clone = IMAGE-482002/clone_end = 3/gb = AI326963/ | 276.7 | P | 302.9 | P | 287.6 | P |
| | | gi = 4061392/ug = Mm 42609/len = 479/STRA = rev | | | | | | |
| 7 | 93301_at | Cluster Incl AW124624 UI-M-BH2 1-apw-b-05-0-UI s1 Mus musculus cDNA, 3 end/clone = UI-M-BH2 1-apw-b-05-0-UI/ | 171 | P | 116.3 | A | 94.6 | P |
| | | clone_end = 3/gb = AW124624/gi = 6100154/ug = Mm 18230/len = 416/STRA = for | | | | | | |
| 7 | 93563_s_at | Cluster Incl AB017202: Mus musculus mRNA for entactin-2, complete cds/cds = (43,4254)/gb = AB017202/gi = 3513367/ | 124.6 | P | 16.7 | M | 28.5 | A |
| | | ug = Mm 20348/len = 5094/STRA = for | | | | | | |
| 7 | 96618_at | Cluster Incl AW045637.UI-M-BH1-alg-f-02-0-UI s1 Mus musculus cDNA, 3 end/clone = UI-M-BH1-alg-f-02-0-UI/ | 139.4 | P | 95.6 | A | 148.5 | P |
| | | clone_end = 3/gb = AW045637/gi = 5906166/ug = Mm 27131/len = 371/STRA = for | | | | | | |
| 7 | 96640_at | Cluster Incl AI644158 vw55c12 x1 Mus musculus cDNA, 3 end/clone = IMAGE-1247734/clone_end = 3/gb = AI644158/ | 297.7 | A | 245.4 | P | 286.1 | P |
| | | gi = 4722633/ug = Mm 27274/len = 458/STRA = for | | | | | | |
| 7 | 96843_at | Cluster Incl AF068116 Mus musculus eIF4E-like protein 4E-LP mRNA, complete cds/cds = (0,737)/gb = AF068116/ | 288.8 | A | 184.2 | P | 324.1 | A |
| | | gi = 3201957/ug = Mm 3002/len = 804/STRA = for | | | | | | |
| 7 | 99603_g_at | Cluster Incl AF064088 Mus musculus transcription factor GIF mRNA, complete cds/cds = (109,1548)/gb = AF064088/ | 565.5 | P | 303 | P | 269.8 | P |
| | | gi = 3139151/ug = Mm 4292/len = 3036/STRA = for | | | | | | |
| 7 | 97360_at | Cluster Incl AI841689 UI-M-AO0-acg-h-11-0-UI s1 Mus musculus cDNA, 3 end/clone = UI-M-AO0-acg-h-11-0-UI/ | 117 | P | 48.2 | A | 82.7 | M |
| | | clone_end = 3/gb = AI841689/gi = 5475902/ug = Mm 29658/len = 337/STRA = for | | | | | | |
| 7 | 99930_s_at | Cluster Incl U57325 Mus musculus PS-2short mRNA, complete cds/cds = (124,579)/gb = U57325/gi = 2315275/ | 51.5 | A | 23.1 | A | 80.3 | P |
| | | ug = Mm 4338/len = 1146/STRA = for | | | | | | |
| 7 | 103308_at | Cluster Incl AI450597: mq87e05 x1 Mus musculus cDNA, 3 end/clone = IMAGE-585728/clone_end = 3/gb = AI450597/ | 1690.8 | P | 658.3 | P | 1038.5 | A |
| | | gi = 4298115/ug = Mm 21144/len = 350/STRA = for | | | | | | |
| 7 | 93068_r_at | Cluster Incl U62674 Mus musculus histone H2a 2-615 (H2a-615), and histone H3.2-615 (H3-615) genes, complete cds/ | 286.1 | A | 327 | A | 355.9 | P |
| | | cds = (43,435)/gb = U62674/gi = 1458142/ug = Mm 15686/len = 515/STRA = for | | | | | | |
| 7 | 93328_at | Cluster Incl X57437: Histidine decarboxylase cluster/cds = (74,2062)/gb = X57437/gi = 52903/ug = Mm 18603/len = 2371/ | 195.3 | P | 85.9 | P | 80.7 | P |
| | | STRA = for | | | | | | |
| 7 | 94840_at | Cluster Incl U05837 Hexosaminidase A/cds = (150,1736)/gb = U05837/gi = 2182207/ug = Mm 2284/len = 1960/STRA = for | 218.3 | P | 127.8 | A | 93.9 | M |
| 7 | 103433_at | Cluster Incl AI846077 UI-M-AN1-afj-a-02-0-UI s2 Mus musculus cDNA, 3 end/clone = UI-M-AN1-afj-a-02-0-UI/ | 299.3 | P | 85.4 | M | 251.7 | P |
| | | clone_end = 3/gb = AI846077/gi = 5490023/ug = Mm 19065/len = 296/STRA = for | | | | | | |
| 7 | 103563_at | Cluster Incl AW125713 UI-M-BH2 2-aql-c-12-0-UI s1 Mus musculus cDNA, 3 end/clone = UI-M-BH2 2-aql-c-12-0-UI/ | 42.2 | A | −27.4 | A | 110.1 | P |
| | | clone_end = 3/gb = AW125713/gi = 6101283/ug = Mm 19126/len = 445/STRA = for | | | | | | |
| 7 | 104516_at | Cluster Incl U82758 Mus musculus lung-specific membrane protein mRNA, complete cds/cds = (21,677)/gb = U82758/ | 371.3 | P | 96.8 | A | 229.2 | P |
| | | gi = 3342229/ug = Mm 22768/len = 1319/STRA = for | | | | | | |
| 7 | 102926_at | Cluster Incl AB008833 Mus musculus mRNA for GFRalpha-3, complete cds/cds = (45,1238)/gb = AB008833/gi = 2627159/ | 472.9 | P | 380.2 | M | 456.8 | A |
| | | ug = Mm.16520/len = 1867/STRA = for | | | | | | |
| 7 | 92731_at | Cluster Incl X83601: Pentaxin related gene/cds = (99,1244)/gb = X83601/gi = 1181243/ug = Mm 4663/len = 1841/STRA = for | 53.7 | P | 84.7 | P | 61.6 | M |
| 7 | 94761_at | Cluster Incl X70058: Small Inducible cytokine A7/cds = (70,342)/gb = X70058/gi = 437872/ug = Mm 16091/len = 774/ | 292.6 | P | 71.4 | A | 108.5 | P |
| | | STRA = for | | | | | | |
| 7 | 102100_f_at | Cluster Incl AI845835: UI-M-AO1-aeg-d-01-0-UI s1 Mus musculus cDNA, 3 end/clone = UI-M-AO1-aeg-d-01-0-UI/ | 60.2 | P | 32.1 | A | 40.1 | P |
| | | clone_end = 3/gb = AI845835/gi = 5489741/ug = Mm 42439/len = 269/STRA = rev | | | | | | |
| 7 | 100521_at | Cluster Incl D32137 Mouse mRNA for oviducl-specific glycoprotein (MOGP), complete cds/cds = (14,2179)/gb = D32137/ | 158.1 | P | 93.9 | P | 102.9 | P |
| | | gi = 806523/ug = Mm 5110/len = 2525/STRA = rev | | | | | | |
| 7 | 96131_at | Cluster Incl AI840116: UI-M-AL0-abt-h-05-0-UI s1 Mus musculus cDNA, 3 end/clone = UI-M-AL0-abt-h-05-0-UI/ | 365.5 | P | 302.6 | A | 272.5 | M |
| | | clone_end = 3/gb = AI840116/gi = 5474329/ug = Mm.28140/len = 348/STRA = for | | | | | | |
| 7 | 104508_at | Cluster Incl AA756506 vv53h12 r1 Mus musculus cDNA, 5 end/clone = IMAGE-1226183/clone_end = 5/gb = AA756506/ | 292.7 | A | 167.5 | A | 203.7 | P |
| | | gi = 2803704/ug = Mm 30805/len = 473/STRA = for | | | | | | |
| 7 | 104647_at | Cluster Incl M88242: Mouse glucocortoid-regulated inflammatory prostaglandin G/H synthase (griPGHS) mRNA, complete | 199.4 | P | 7.1 | A | 87.7 | P |
| | | cds/cds = (100,1914)/gb = M88242/gi = 193637/ug = Mm 3137/len = 4152/STRA = for | | | | | | |
| 7 | 93153_at | Cluster Incl AW124433: UI-M-BH2.1-apo-b-10-0-UI.s1 Mus musculus cDNA, 3 end/clone = UI-M-BH2.1-apo-b-10-0-UI/ | 402.8 | P | 314.7 | P | 178.5 | A |
| | | clone_end = 3/gb = AW124433/gi = 6100003/ug = Mm.5258/len = 526/STRA = rev | | | | | | |
| 7 | 95956_at | Cluster Incl C76923: C76923 Mus musculus cDNA, 3 end/clone = J0023A02/clone_end = 3/gb = C76923/gi = 2979405/ | 118.2 | P | 110.5 | A | 88.6 | P |
| | | ug = Mm 24933/len = 429/STRA = rev | | | | | | |
| 7 | 97103_at | Cluster Incl AF031380 Mus musculus KOI-4 gene, partial cds/cds = (0,102)/gb = AF031380/gi = 2623675/ug = Mm 32715/ | 36.9 | P | −59.9 | P | −97.7 | A |
| | | len = 103/STRA = rev | | | | | | |
| 7 | 92549_at | Cluster Incl U97170 Mus musculus protein kinase inhibitor gamma (PKIg) mRNA, complete cds/cds = (217,447)/ | 540.8 | P | 345.1 | A | 445 | P |
| | | gb = U97170/gi = 3004635/ug = Mm 10091/len = 1064/STRA = for | | | | | | |
| 7 | 93985_at | Cluster Incl AW120868: UI-M-BH2.3-aoa-g-07-0-UI s1 Mus musculus cDNA, 3 end/clone = UI-M-BH2.3-aoa-g-07-0-UI/ | 18 | A | −108.6 | P | 14.3 | A |
| | | clone_end = 3/gb = AW120868/gi = 6096201/ug = Mm 21712/len = 284/STRA = for | | | | | | |
| 7 | 96605_at | Cluster Incl AI787183: u86a11 y1 Mus musculus cDNA, 5 end/clone = IMAGE-1889276/clone_end = 5/gb = AI787183/ | 218.4 | A | 65.9 | A | 128.8 | P |
| | | gi = 5334899/ug = Mm 27061/len = 245/STRA = for | | | | | | |
| 7 | 97811_at | Cluster Incl AI844507: UI-M-AJ1-ahe-g-11-0-UI s1 Mus musculus cDNA, 3 end/clone = UI-M-AJ1-ahe-g-11-0-UI/ | 83.2 | M | −103 | A | 72.1 | A |
| | | clone_end = 3/gb = AI844507/gi = 5488413/ug = Mm 28163/len = 299/STRA = for | | | | | | |
| 7 | 94517_r_at | Cluster Incl M55181 Preproenkephalin 2/cds = UNKNOWN/gb = M55181/gi = 201032/ug = Mm 2899/len = 1408/STRA = for | 604.8 | P | 153.2 | A | 364.8 | A |
| 7 | 102326_at | Cluster Incl AB002664 Mus musculus mRNA for p67phox, complete cds/cds = (96,1673)/gb = AB002664/gi = 3061283/ | 127.8 | A | 42.7 | M | 53.6 | A |
| | | ug = Mm.10729/len = 1813/STRA = for | | | | | | |
| 7 | 96203_at | Cluster Incl AI847646.UI-M-AP1-agg-g-07-0-UI s1 Mus musculus cDNA, 3 end/clone = UI-M-AP1-agg-g-07-0-UI/ | 84.2 | P | −33.7 | A | 37.1 | A |
| | | clone_end = 3/gb = AI847646/gi = 5491552/ug = Mm 28623/len = 226/STRA = for | | | | | | |
| 7 | 99486_at | Cluster Incl X55038 Centromere autoantigen B/cds = (0,1799)/gb = X55038/gi = 50381/ug = Mm 41454/len = 1800/STRA = for | 394.9 | P | 163.8 | A | 281.7 | A |
| 7 | 92534_at | Cluster Incl U10551 GTP binding protein (gene overexpressed in skeletal muscle)/cds = (108,995)/gb = U10551/gi = 511464/ | 166.8 | P | 22 | A | 44 | A |
| | | ug = Mm 4362/len = 1981/STRA = for | | | | | | |
| 7 | 92688_at | Cluster Incl X57199 Acid phosphatase 2, lysosomal/cds = (0,1265)/gb = X57199/gi = 52870/ug = Mm 45570/len = 1266/ | 226 | A | 175.6 | M | 199.9 | A |
| | | STRA = for | | | | | | |
| 7 | 93656_g_at | Cluster Incl X95316 M musculus USF1 (exons 2 to 10)/cds = (214,1146)/gb = X95316/gi = 1765921/ug = Mm 8/len = 1902/ | 329.7 | P | 283.9 | A | 282.2 | P |
| | | STRA = for | | | | | | |
| 7 | 94778_at | Cluster Incl U96401: Mus musculus aldehyde dehydrogenase Ahd-2-like mRNA, complete cds/cds = (44,1549)/gb = U96401/ | 275.2 | P | 166.7 | A | 70.3 | A |
| | | gi = 2289239/ug = Mm.16773/len = 2060/STRA = for | | | | | | |
| 7 | 95380_at | Cluster Incl L19057 Non MHC restricted killing associated/cds = (126,1322)/gb = L19057/gi = 309076/ug = Mm 2299/ | 70.8 | P | 61.9 | A | 49.2 | A |
| | | len = 1930/STRA = for | | | | | | |
| 7 | 99835_at | Cluster Incl AF017128 Fos-like antigen 1/cds = (253,1074)/gb = AF017128/gi = 2407190/ug = Mm 6215/len = 1705/STRA = for | 611.1 | P | 148.1 | A | 57.6 | P |
| 8 | 92574_at | Cluster Incl AI851046 UI-M-BH0-ajv-f-05-0-UI s1 Mus musculus cDNA, 3 end/clone = UI-M-BH0-ajv-f-05-0-UI/ | 869.4 | P | 691.2 | P | 906.8 | P |
| | | clone_end = 3/gb = AI851046/gi = 5494952/ug = Mm 10406/len = 475/STRA = for | | | | | | |
| 8 | 92615_at | Cluster Incl AI853615 UI-M-BH0-ajp-f-09-0-UI s1 Mus musculus cDNA, 3 end/clone = UI-M-BH0-ajp-f-09-0-UI/ | 1037.2 | P | 712.2 | P | 1102.8 | P |
| | | clone_end = 3/gb = AI853615/gi = 5497521/ug = Mm 1103/len = 542/STRA = for | | | | | | |
| 8 | 92625_at | Cluster Incl X68193: M. musculus mRNA for nucleoside diphoshate kinase B/cds = (19,477)/gb = X68193/gi = 53353/ | 3419 | P | 2939.2 | P | 3786.9 | P |
| | | ug = Mm.1125/len = 612/STRA = for | | | | | | |
| 8 | 93026_at | Cluster Incl AW124337.UI-M-BH2 1-apq-f-08-0-UI.s1 Mus musculus cDNA, 3 end/clone = UI-M-BH2.1-apq-f-08-0-UI/ | 418.6 | P | 975.9 | P | 1024.2 | P |
| | | clone_end = 3/gb = AW124337/gi = 6099867/ug = Mm 14796/len = 243/STRA = rev | | | | | | |
| 8 | 93071_at | Cluster Incl X99644: M musculus mRNA for TIF1 beta protein/cds = (168,2672)/gb = X99644/gi = 1480113/ug = Mm 15701/ | 1771 | P | 1808.4 | P | 2048.2 | P |
| | | len = 2858/STRA = for | | | | | | |
| 8 | 93270_at | Cluster Incl AI839918 UI-M-AH0-acw-f-01-0-UI s1 Mus musculus cDNA, 3 end/clone = UI-M-AH0-acw-f-01-0-UI/ | 827.5 | P | 754 | P | 837.7 | P |
| | | clone_end = 3/gb = AI839918/gi = 5474131/ug = Mm 17403/len = 429/STRA = for | | | | | | |
| 8 | 93295_at | Cluster Incl Z31555 M musculus (129/Sv) Ccte mRNA for CCT (chaperonin containing TCP-1) epsilon subunit/ | 1957.2 | P | 1767.1 | P | 2196.6 | P |
| | | cds = (99,1724)/gb = Z31555/gi = 468549/ug = Mm 1813/len = 1769/STRA = for | | | | | | |
| 8 | 93305_f_at | Cluster Incl AF053724: Mus musculus endobrevin mRNA, complete cds/cds = (154,459)/gb = AF053724/gi = 2982367/ | 828 | P | 556.9 | P | 930.7 | P |
| | | ug = Mm 1838/len = 597/STRA = for | | | | | | |
| 8 | 93340_f_at | Cluster Incl AF043120: Mus musculus beta prime coatomer protein mRNA, partial cds/cds = (0,1352)/gb = AF043120/ | 268.3 | P | 278.2 | P | 354.5 | P |
| | | gi = 2809536/ug = Mm.18729/len = 1615/STRA = for | | | | | | |
| 8 | 93358_at | Cluster Incl AI836451 UI-M-AI0-aak-e-05-0-UI.s1 Mus musculus cDNA, 3 end/clone = UI-M-AI0-aak-e-05-0-UI/ | 860.9 | P | 974.5 | P | 914.3 | P |
| | | clone_end = 3/gb = AI836451/gi = 5470664/ug = Mm.18924/len = 368/STRA = for | | | | | | |
| 8 | 93533_at | Cluster Incl AI853651 UI-M-BH0-ajq-b-03-0-UI s1 Mus musculus cDNA, 3 end/clone = UI-M-BH0-ajq-b-03-0-UI/ | 341.5 | P | 374.8 | P | 415.1 | P |
| | | clone_end = 3/gb = AI853651/gi = 5497557/ug = Mm 1985/len = 445/STRA = for | | | | | | |
| 8 | 93557_at | Cluster Incl U43285 Selenophosphate synthetase 2/cds = (137,1495)/gb = U43285/gi = 1815623/ug = Mm.20294/len = 2202/ | 110 | P | 170.8 | P | 158.8 | P |
| | | STRA = for | | | | | | |
| 8 | 93581_at | Cluster Incl AI845121 UI-M-BG0-ahs-b-12-0-UI.s1 Mus musculus cDNA, 3 end/clone = UI-M-BG0-ahs-b-12-0-UI/ | 1784.9 | P | 1337.3 | P | 1906.8 | P |
| | | clone_end = 3/gb = AI845121/gi = 5489027/ug = Mm 2060/len = 361/STRA = for | | | | | | |
| 8 | 93593_f_at | Cluster Incl U87948 Epithelial membrane protein 3/cds = (201,692)/gb = U87948/gi = 2358253/ug = Mm 20829/len = 695/ | 1357.2 | P | 976.7 | P | 1148.9 | P |
| | | STRA = for | | | | | | |
| 8 | 93596_i_at | Cluster Incl AA624586 vn82e11.r1 Mus musculus cDNA, 5 end/clone = IMAGE-1038476/clone_end = 5/gb = AA624588/ | 1242.3 | P | 742 | P | 1288.2 | P |
| | | gi = 2528462/ug = Mm 20841/len = 178/STRA = for | | | | | | |
| 8 | 93734_i_at | Cluster Incl D49686: Mus musculus mRNA for Tat binding protein-1, complete cds/cds = (176,1504)/gb = D49686/ | 542.2 | P | 482.2 | P | 571 | P |
| | | gi = 3510276/ug = Mm.20946/len = 1569/STRA = for | | | | | | |
| 8 | 93735_f_at | Cluster Incl D49686: Mus musculus mRNA for Tat binding protein-1, complete cds/cds = (176,1504)/gb = D49686/ | 1103.1 | P | 943.9 | P | 1198.1 | P |
| | | gi = 3510276/ug = Mm.20946/len = 1569/STRA = for | | | | | | |
| 8 | 93760_at | Cluster Incl AI838759 UI-M-AO0-acf-f-02-0-UI s1 Mus musculus cDNA, 3 end/clone = UI-M-AO0-acf-f-02-0-UI/ | 933.7 | P | 896.7 | P | 935.6 | P |
| | | clone_end = 3/gb = AI838759/gi = 5472972/ug = Mm.21149/len = 460/STRA = for | | | | | | |
| 8 | 93774_at | Cluster Incl AW049040 UI-M-BH1-amp-h-08-0-UI s1 Mus musculus cDNA, 3 end/clone = UI-M-BH1-amp-h-08-0-UI/ | 648 | P | 657.4 | P | 749 | P |
| | | clone_end = 3/gb = AW049040/gi = 5909569/ug = Mm.21207/len = 260/STRA = for | | | | | | |
| 8 | 93789_a_at | Cluster Incl AF038848: Mus musculus transcriptional repressor SIN3B (Sin3B) mRNA, short form, complete cds/ | 1020.9 | P | 867.2 | P | 1107.3 | P |
| | | cds = (20,901)/gb = AF038848/gi = 2921546/ug = Mm.2137/len = 1056/STRA = for | | | | | | |
| 8 | 93823_at | Cluster Incl AI844864: UI-M-AH1-agx-d-05-0-UI.s1 Mus musculus cDNA, 3 end/clone = UI-M-AH1-agx-d-05-0-UI/ | 147 | P | 135.8 | P | 125.2 | P |
| | | clone_end = 3/gb = AI844864/gi = 5488735/ug = Mm 21534/len = 527/STRA = for | | | | | | |
| 8 | 93968_at | Cluster Incl AA870917: vq28a06.r1 Mus musculus cDNA, 5 end/clone = IMAGE-1095538/clone_end = 5/gb = AA870917/ | 608.2 | P | 752.9 | P | 840.6 | P |
| | | gi = 2968382/ug = Mm 21650/len = 454/STRA = for | | | | | | |
| 8 | 93972_at | Cluster Incl AI8374493 UI-M-AL0-abr-f-12-0-UI.s1 Mus musculus cDNA, 3 end/clone = UI-M-AL0-abr-f-12-0-UI/clone_end = 3 | 1458.4 | P | 1180.8 | P | 1625.5 | P |
| | | gb = AI83749/gi = 5471706/ug = Mm.21669/len = 341/STRA = for | | | | | | |
| 8 | 93973_at | Cluster Incl AI854624 UI-M-BH0-ake-e-06-0-UI.a1 Mus musculus cDNA, 3 end/clone = UI-M-BH0-ake-e-06-0-UI/ | 939.4 | P | 901.2 | P | 1159.4 | P |
| | | clone_end = 3/gb = AI854624/gi = 5498530/ug = Mm 21671/len = 474/STRA = for | | | | | | |
| 8 | 93988_at | Cluster Incl AI836676 UI-M-AP0-abh-g-06-0-UI a1 Mus musculus cDNA, 3 end/clone = UI-M-AP0-abh-g-06-0-UI/ | 1195.5 | P | 918.3 | P | 1432.4 | P |
| | | clone_end = 3/gb = AI836676/gi = 5470889/ug = Mm 21728/len = 471/STRA = for | | | | | | |
| 8 | 94001_at | Cluster Incl U65735 Mus musculus eiav G homolog (MelG) mRNA, complete cds/cds = (0,980)/gb = U65735/gi = 1655900/ | 536.8 | P | 820.6 | P | 860 | P |
| | | ug = Mm 21766/len = 981/STRA = for | | | | | | |
| 8 | 94004_at | Cluster Incl Z19543: M musculus h2-calponin cDNA/cds = (102,1019)/gb = Z51143/ug = Mm 21776/len = 1821/ | 440.1 | P | 523.8 | P | 392.6 | P |
| | | STRA = for | | | | | | |
| 8 | 94025_at | Cluster Incl AW045339: UI-M-BH1-ako-a-09-0-UI s1 Mus musculus cDNA, 3 end/clone = UI-M-BH1-ako-a-09-0-UI/ | 698.1 | P | 844.6 | P | 1207.6 | P |
| | | clone_end = 3/gb = AW045339/gi = 5905868/ug = Mm 21874/len = 430/STRA = for | | | | | | |
| 8 | 94061_at | Cluster Incl M13018 Cysteine rich Intestinal protein/cds = (61,294)/gb = M13018/gi = 192725/ug = Mm 22049/len = 404/ | 692.5 | P | 79 | P | 298.9 | P |
| | | STRA = for | | | | | | |
| 8 | 94062_at | Cluster Incl AI847609: UI-M-AP1-agg-c-11-0-UI s1 Mus musculus cDNA, 3 end/clone = UI-M-AP1-agg-c-11-0-UI/ | 2290.5 | P | 1503.1 | P | 2185.7 | P |
| | | clone_end = 3/gb = AI847609/gi = 5491515/ug = Mm 2206/len = 356/STRA = for | | | | | | |
| 8 | 94078_at | Cluster Incl AW046239: UI-M-BH1-ala-b-12-0-UI s1 Mus musculus cDNA, 3 end/clone = UI-M-BH1-ala-b-12-0-UI/ | 1996.3 | P | 1803.8 | P | 2219.3 | P |
| | | clone_end = 3/gb = AW046239/gi = 5906768/ug = Mm 22138/len = 363/STRA = for | | | | | | |
| 8 | 94219_at | Cluster Incl AI853269: UI-M-BH0-ajl-c-11-0-UI s1 Mus musculus cDNA, 3 end/clone = UI-M-BH0-ajl-c-11-0-UI/clone_end = 3/ | 1195.6 | P | 1285.2 | P | 1264.7 | P |
| | | gb = AI853269/gi = 5479175/ug = Mm 22233/len = 375/STRA = for | | | | | | |
| 8 | 94263_f_at | Cluster Incl D85570 Proteasome (prosome, macropain) subunit, beta type 7/cds = (20,853)/gb = D85570/gi = 2547069/ | 2063.5 | P | 1659.8 | P | 1751 | P |
| | | ug = Mm 2246/len = 991/STRA = for | | | | | | |
| 8 | 94821_at | Cluster Incl AW123880 UI-M-BH2.1-app-e-07-0-UI.s1 Mus musculus cDNA, 3 end/clone = UI-M-BH2.1-app-e-07-0-UI/ | 809.1 | P | 1241.3 | P | 1221.4 | P |
| | | clone_end = 3/gb = W123880/gi = 6099410/ug = Mm 22718/len = 448/STRA = for | | | | | | |
| 8 | 94841_at | Cluster Incl AW148997 UI-M-BH1-amp-c-10-0-UI s1 Mus musculus cDNA, 3 end/clone = UI-M-BH1-amp-c-10-0-UI/ | 914 | P | 906.7 | P | 1122.8 | P |
| | | clone_end = 3/gb = AW048997/gi = 5909526/ug = Mm 2287/len = 486/STRA = for | | | | | | |
| 8 | 94875_at | Cluster Incl AI838915 UI-M-AO0-ace-a-11-0-UI s1 Mus musculus cDNA, 3 end/clone = UI-M-AO0-ace-a-11-0-UI/ | 346 | P | 335.5 | P | 458 | P |
| | | clone_end = 3/gb = AI838915/gi = 5473128/ug = Mm 23825/len = 398/STRA = for | | | | | | |
| 8 | 94876_f_at | Cluster Incl AI849207: UI-M-AJ1-agz-a-02-0-UI s1 Mus musculus cDNA, 3 end/clone = UI-M-AJ1-agz-a-02-0-UI/ | 533.6 | P | 758.2 | P | 839.3 | P |
| | | clone_end = 3/gb = AI849207/gi = 5493113/ug = Mm 23836/len = 449/STRA = for | | | | | | |
| 8 | 94897_at | Cluster Incl D87896 Glutathione peroxidase 4/cds = (144,737)/gb = D87896/gi = 2522258/ug = Mm 2400/len = 958/STRA = for | 569.6 | P | 342.5 | P | 677 | P |
| 8 | 94907_f_at | Cluster Incl AW045632: UI-M-BH1-alg-e-06-0-UI s1 Mus musculus cDNA, 3 end/clone = UI-M-BH1-alg-e-06-0-UI/ | 966.7 | P | 899.2 | P | 909.9 | P |
| | | clone _end = 3/gb = AW045632/gi = 5906161/ug = Mm 24091/len = 248/STRA = for | | | | | | |
| 8 | 94909_at | Cluster Incl AW046919: UI-M-BH1-akl-g-03-0-UI s1 Mus musculus cDNA, 3 end/clone = UI-M-BH1-akl-g-03-0-UI/ | 351.9 | P | 287.2 | P | 329 | P |
| | | clone _end = 3/gb = AW046919/gi = 5907448/ug = Mm 24104/len = 440/STRA = for | | | | | | |
| 8 | 94912_at | Cluster Incl AI835592: UI-M-AI0-app-a-05-0-UI.s1 Mus musculus cDNA, 3 end/clone = UI-M-AI0-aap-a-05-0-UI/ | 531.8 | P | 524.7 | P | 636.8 | P |
| | | clone_end = 3/gb = AI835592/gi = 5469805/ug = Mm 24108/len = 229/STRA = for | | | | | | |
| 8 | 94913_at | Cluster Incl U65313 Mus musculus ras-GTPase-activating protein SH3-domain binding protein (G3BP) mRNA, complete | 375 | P | 491.3 | P | 500.3 | P |
| | | cds/cds = (164,1612)/gb = U65313/gi = 2961438/ug = Mm.2411/len = 1800/STRA = for | | | | | | |
| 8 | 95409_at | Cluster Incl AW047671: UI-M-BH1-alo-c-06-0-UI s1 Mus musculus cDNA, 3 end/clone = UI-M-BH1-alo-c-06-0-UI/ | 473.2 | P | 490.1 | P | 564.3 | P |
| | | clone_end = 3/gb = AW047671/gi = 5908200/ug = Mm 24228/len = 339/STRA = for | | | | | | |
| 8 | 95424_at | Cluster Incl AW120986: UI-M-BH2 3-aod-f-01-0-UI s1 Mus musculus cDNA, 3 end/clone = UI-M-BH2.3-aod-f-01-0-UI/ | 3163.1 | P | 2696.3 | P | 3591.2 | P |
| | | clone_end = 3/gb = AW120986/gi = 609631/ug = Mm 24433/len = 498/STRA = for | | | | | | |
| 8 | 95441_at | Cluster Incl AB201122: Mus musculus Tim23 mRNA for translocase of inner mitochondrial membrane, complete cds/ | 2214.9 | P | 2267.5 | P | 2494.3 | P |
| | | cds = (123,752)/gb = AB021122/gi = 4996327/ug = Mm 24565/len = 1142/STRA = for | | | | | | |
| 8 | 95462_at | Cluster Incl AW060951 UI-M-BH1-anv-f-08-0-UI s1 Mus musculus cDNA, 3 end/clone = UI-M-BH1-anv-f-08-0-UI/ | 1087.1 | P | 940.1 | P | 1154.2 | P |
| | | clone_end = 3/gb = AW060951/gi = 6008702/ug = Mm 24745/len = 228/STRA = for | | | | | | |
| 8 | 95517_i_at | Cluster Incl AI846338: UI-M-AN1-afa-c-01-0-UI.s1 Mus musculus cDNA, 3 end/clone = UI-M-AN1-afa-c-10-0-UI/ | 587.9 | P | 643.3 | P | 766.3 | P |
| | | clone_end = 3/gb = AI846338/gi = 5490244/ug = Mm 25308/len = 377/STRA = for | | | | | | |
| 8 | 96008_at | Cluster Incl U81052: Mus musculus Defender against Apoptotic Death (Dad1) gene/cds = (25,366)/gb = U81052/gi = 1905978/ | 1327.3 | P | 1543.4 | P | 1429.2 | P |
| | | ug = Mm 2547/len = 406/STRA = for | | | | | | |
| 8 | 96611_at | Cluster Incl AI843074 UI-M-AK1-aes-s-11-0-UI s1 Mus musculus cDNA, 3 end/clone = UI-M-AK1-aes-a-11-0-UI/ | 844.1 | P | 596.8 | P | 828.8 | P |
| | | clone_end = 3/gb = AI843074/gi = 5477287/ug = Mm 27087/len = 253/STRA = for | | | | | | |
| 8 | 96666_at | Cluster Incl U57692 N-terminal Asn amidase/cds = (33,965)/gb = U57692/gi = 1373366/ug = Mm 27378/len = 1101/STRA = for | −21.8 | P | −88.7 | P | 153.2 | P |
| 8 | 96686_i_at | Cluster Incl AI853864: UI-M-BH0-ajr-f-08-0-UI s1 Mus musculus cDNA, 3 end/clone = UI-M-BH0-ajr-f-08-0-UI/clone_end = 3/ | 1433.8 | P | 1267.6 | P | 1516.5 | P |
| | | gb = AI853864/gi = 5497770 ug = Mm.27477/len = 461/STRA = for | | | | | | |
| 8 | 96687_f_at | Cluster Incl AI853864 UI-M-BH0-ajr-f-08-0-UI s1 Mus musculus cDNA, 3 end/clone = UI-M-BH0-ajr-f-08-0-UI/clone_end = 3/ | 702.6 | P | 580 | P | 894.5 | P |
| | | gb = AI853864/gi = 5497770 ug = Mm 27477/len = 461/STRA = for | | | | | | |
| 8 | 97203_at | Cluster Incl X61399: Mouse F52 mRNA for a novel protein/cds = UNKNOWN/gb = X61399/gi = 50943/ug = Mm 2769/ | 1359.9 | P | 1190.2 | P | 1191.8 | P |
| | | len = 1584/STRA = for | | | | | | |
| 8 | 97204_s_at | Cluster Incl AI850983: UI-M-BH0-akd-h-08-0-UI s1 Mus musculus cDNA, 3 end/clone = UI-M-BH0-akd-h-08-0-UI/ | 222 | P | 199.9 | P | 225 | P |
| | | clone_end = 3/gb = AI850983/gi = 5494889/ug = Mm 27692/len = 265/STRA = for | | | | | | |
| 8 | 97207_f_at | Cluster Incl AI875934: uj64h06 x1 Mus musculus cDNA, 3 end/clone = IMAGE-1924763/clone_end = 3/gb = AI675934/ | 2328.3 | P | 1823.8 | P | 2002.6 | P |
| | | gi = 5549983/ug = Mm 27700/len = 330/STRA = for | | | | | | |
| 8 | 97248_at | Cluster Incl X61431: M. musculus mRNA for diazepam-binding inhibitor/cds = (62,325)/gb = X61431/gi = 50712/ug = Mm.2785/ | 1771.6 | P | 1614.4 | P | 2085.9 | P |
| | | len = 390/STRA = for | | | | | | |
| 8 | 97911_at | Cluster Incl AI835950-UI-M-AI0-aao-f-11-0-UI.s1 Mus musculus cDNA. 3 end/clone = UI-M-AI0-aao-f-11-0-UI/clone_end = 3/ | 561.3 | P | 466.8 | P | 553.4 | P |
| | | gb = AI835950/gi = 5470163/ug = Mm 28482/len = 380/STRA = for | | | | | | |
| 8 | 98875_at | Cluster Incl AW121160: UI-M-BH2 3-aoh-g-08-0-UI s1 Mus musculus cDNA, 3 end/clone = UI-M-BH2 3-aoh-g-08-0-UI/ | 96.1 | P | 142.2 | P | 89.1 | P |
| | | clone_end = 3/gb = AW121160/gi = 6096493/ug = Mm 28492/len = 139/STRA = for | | | | | | |
| 8 | 94494 _at | Cluster Incl AI840856 UI-M-AH0-adb-g-11-0-UI s1 Mus musculus cDNA, 3 end/clone = UI-M-AH0-adb-g-11-0-UI/ | 307.4 | P | 396.2 | P | 294.4 | P |
| | | clone_end = 3/gb = AI840856/gi = 5475069/ug = Mm 28922/len = 274/STRA = for | | | | | | |
| 8 | 94506 _at | Cluster Incl AI853113 UI-M-BH0-ajf-d-10-0-UI s1 Mus musculus cDNA, 3 end/clone = UI-M-BH0-ajf-d-10-0-UI/ | 321.8 | P | 252.8 | P | 310.1 | P |
| | | clone_end = 3/gb = AI853113/gi = 5497019/ug = Mm.28961/len = 205/STRA = for | | | | | | |
| 8 | 94522 _at | Cluster Incl AF098508 Mus musculus dynaclin light chain (P24) mRNA, complete cds/cds = (12,572)/gb = AF098508/ | 428.3 | P | 474.3 | P | 582.3 | P |
| | | gi = 4323147/ug = Mm.29025/len = 819/STRA = for | | | | | | |
| 8 | 94550 _at | Cluster Incl AW121324 UI-M-BH2.2-aom-a-06-0-UI s1 Mus musculus cDNA, 3 end/clone = UI-M-BH2.2-aom-a-06-0-UI/ | 193.8 | P | 353.8 | P | 561.6 | P |
| | | clone_end = 3/gb = AW121324/gi = 6096657/ug = Mm 29079/len = 541/STRA = for | | | | | | |
| 8 | 95042 _at | Cluster Incl AI839770 UI-M-AN0-acq-f-01-0-UI s1 Mus musculus cDNA, 3 end/clone = UI-M-AN0-acq-f-01-0-UI/ | 495.9 | P | 490.4 | P | 571.2 | P |
| | | clone_end = 3/gb = AI839770/gi = 5474023/ug = Mm 29118/len = 148/STRA = for | | | | | | |
| 8 | 95066 _at | Cluster Incl U67611 Mouse transaidolase gene mRNA, complete cds/cds = (5,1018)/gb = U67611/gi = 2589165/ | 331.7 | P | 195.2 | P | 303.4 | P |
| | | ug = Mm 29132/len = 1155/STRA = for | | | | | | |
| 8 | 95091_at | Cluster Incl AI839895.UI-M-AH0-acw-d-02-0-UI s1 Mus musculus cDNA, 3 end/clone = UI-M-AH0-acw-d-02-0-UI/ | 606.2 | P | 455.3 | P | 532.8 | P |
| | | clone_end = 3/gb = AI839895/gi = 5474108/ug = Mm 29296/len = 446/STRA = for | | | | | | |
| 8 | 95097 _at | Cluster Incl AI851227: UI-M-BH0-ajx-h-07-0-UI s1 Mus musculus cDNA, 3 end/clone = UI-M-BH0-ajx-h-07-0-UI/ | 565.3 | P | 537.9 | P | 665.7 | P |
| | | clone_end = 3/gb = AI851227/gi = 5495133/ug = Mm 29317/len = 354/STRA = for | | | | | | |
| 8 | 95131_f_at | Cluster Incl AI852592: UI-M-BH0-aiu-b-11-0-UI s1 Mus musculus cDNA, 3 end/clone = UI-M-BH0-aiu-b-11-0-UI/ | 1979.1 | P | 1420 | P | 2127.6 | P |
| | | clone_end = 3/gb = AI852592/gi = 5496498/ug = Mm 29415/len = 392/STRA = rev | | | | | | |
| 8 | 95137 _at | Cluster Incl AI852985: UI-M-BH0-ajn-a-04-0-UI s1 Mus musculus cDNA, 3 end/clone = UI-M-BH0-ajn-a-04-0-UI/ | 560.5 | P | 576.3 | P | 645.5 | P |
| | | clone_end = 3/gb = AI852985/gi = 5496891/ug = Mm 29431/len = 428/STRA = for | | | | | | |
| 8 | 95147 _at | Clusler Incl AI843795: UI-M-AN1-afc-e-05-0-UI.s1 Mus musculus cDNA, 3 end/clone = UI-M-AN1-afc-e-05-0-UI/ | 1862 | P | 1251.3 | P | 2105 | P |
| | | clone_end = 3/gb = AI843795/gi = 5478008/ug = Mm 29456/len = 462/STRA = for | | | | | | |
| 8 | 95149_at | Cluster Incl AW121088: UI-M-BH2 3-aof-h-04-0-UI s1 Mus musculus cDNA, 3 end/clone = UI-M-BH2.3-aof-h-04-0-UI/ | 561.2 | P | 596.3 | P | 569.9 | P |
| | | clone_end = 3/gb = AW121088/gi = 6096421/ug = Mm 29473/len = 505/STRA = for | | | | | | |
| 8 | 95659_at | Cluster Incl AF041476 Mus musculus BAF53a (Baf53a) mRNA, complete cds/cds = (130,1419)/gb = AF041476/gi = 4001804/ | 334.7 | P | 430.4 | P | 366.2 | P |
| | | ug = Mm 29546/len = 1770/STRA = for | | | | | | |
| 8 | 95690_at | Cluster Incl AW047363 UI-M-BH1-ame-c-09-0-UI s1 Mus musculus cDNA, 3 end/clone = UI-M-BH1-ame-c-09-0-UI/ | 235 | P | 317.2 | P | 399 | P |
| | | clone_end = 3/gb = AW047363/gi = 5907892/ug = Mm 29651/len = 398/STRA = for | | | | | | |
| 8 | 95696_at | Cluster Incl AI840882: UI-M-AM0-adm-b-04-0-UI.s1 Mus musculus cDNA, 3 end/clone = UI-M-AM0-adm-b-04-0-UI/ | 1679.5 | P | 1374.2 | P | 1658.3 | P |
| | | clone_end = 3/gb = AI840882/gi = 5475095/ug = Mm 29675/len = 400/STRA = for | | | | | | |
| 8 | 95700_f_at | Cluster Incl AI848094 UI-M-AP1-agk-d-05-0-UI s1 Mus musculus cDNA, 3 end/clone = UI-M-AP1-agk-d-05-0-UI/ | 254.8 | P | 263.8 | P | 327.6 | P |
| | | clone_end = 3/gb = AI848094/gi = 5492000/ug = Mm 29685/len = 399/STRA = for | | | | | | |
| 8 | 95703_at | Cluster Incl AB024303 Mus musculus mRNA similar to human Sua1, complete cds/cds = (34,1086)/gb = AB024303/ | 496.4 | P | 541.5 | P | 647.9 | P |
| | | gi = 5689241/ug = Mm 29698/len = 1870/STRA = for | | | | | | |
| 8 | 95707_at | Cluster Incl AA615853 Vo94104.r1 Mus musculus cDNA, 5 end/clone = IMAGE-1066783/clone_end = 5/gb = AA615853/ | 699.6 | P | 379.2 | P | 579.6 | P |
| | | gi = 2503081/ug = Mm 29700/len = 534/STRA = for | | | | | | |
| 8 | 95714_at | Cluster Incl AI226264: uf06a12y1 Mus musculus cDNA, 5 end/clone = IMAGE-1510558/clone_end = 5/gb = AI226264/ | 601.4 | P | 691 | P | 685.7 | P |
| | | gi = 3809317/ug = Mm 29712/len = 465/STRA = for | | | | | | |
| 8 | 95716_at | Cluster Incl AW125041: UI-M-BH2 1-apx-b-01-0-UI s1 Mus musculus cDNA, 3 end/clone = UI-M-BH2 1-apx-b-01-0-UI/ | 2576.6 | P | 2611.5 | P | 2723.5 | P |
| | | clone_end = 3/gb = AW125041/gi = 6100571/ug = Mm 29717/len = 227/STRA = for | | | | | | |
| 8 | 95718_f_at | Cluster Incl AW060158 UI-M-BH1-amu-b-10-0-UI s1 Mus musculus cDNA, 3 end/clone = UI-M-BH1-amu-b-10-0-UI/ | 2714.7 | P | 2403.1 | P | 2792.9 | P |
| | | clone_end = 3/gb = AW060158/gi = 6007924/ug = Mm 29722/len = 360/STRA = for | | | | | | |
| 8 | 95736_at | Cluster Incl AI847546: UI-M-AM1-afv-f-01-0-UI s1 Mus musculus cDNA, 3 end/clone = UI-M-AM1-afv-f-01-0-UI/ | 562.6 | P | 529.7 | P | 550.4 | P |
| | | clone_end = 3/gb = AI847546/gi = 5491452/ug = Mm 29749/len = 452/STRA = for | | | | | | |
| 8 | 95742_at | Cluster Incl AW045451: UI-M-BH1-akr-g-10-0-UI s1 Mus musculus cDNA, 3 end/clone = UI-M-BH1-akr-g-10-0-UI/ | 469.9 | P | 501.9 | P | 524 | P |
| | | clone_end = 3/gb = AW045451/gi = 5906020/ug = Mm 29760/len = 340/STRA = for | | | | | | |
| 8 | 95760_at | Cluster Incl AI848730: UI-M-AJ1-ahf-d-04-0-UI.s1 Mus musculus cDNA, 3 end/clone = UI-M-AJ1-ahf-d-04-0-UI/ | 351.4 | P | 224.5 | P | 299.7 | P |
| | | clone_end = 3/gb = AI848730/gi = 5492636/ug = Mm 29801/len = 477/STRA = for | | | | | | |
| 8 | 96249_at | Cluster Incl AW122105: UI-M-BH2 2-aoo-h-12-0-UI.s1 Mus musculus cDNA, 3 end/clone = UI-M-BH2.2-aoo-h-12-0-UI/ | 659.4 | P | 540.2 | P | 680.7 | P |
| | | clone_end = 3/gb = AW122105/gi = 6097518/ug = Mm.29812/len = 357/STRA = rev | | | | | | |
| 8 | 96258_at | Cluster Incl AI843448. UI-M-AQ1-aec-e-01-0-UI s1 Mus musculus cDNA, 3 end/clone = UI-M-AQ1-aec-e-01-0-UI/ | 1357.2 | P | 1242.4 | P | 1468.3 | P |
| | | clone_end = 3/gb = AI843448/gi = 5477661/ug = Mm 29823/len = 382/STRA = for | | | | | | |
| 8 | 96276_r_at | Cluster Incl AI843327: UI-M-AQ1-adz-a-05-0-UI s1 Mus musculus cDNA, 3 end/clone = UI-M-AQ1-adz-a-05-0-UI/ | 245.9 | P | 308.3 | P | 309.5 | P |
| | | clone_end = 3/gb = AI843327/gi = 5477540/ug = Mm 2986/len = 339/STRA = for | | | | | | |
| 8 | 96291_f_at | Cluster Incl AI835847 UI-M-AI0-aat-b-07-0-UI s1 Mus musculus cDNA, 3 end/clone = UI-M-AI0-aat-b-07-0-UI/clone_end = 3/ | 1009.4 | P | 1046.2 | P | 1141.1 | P |
| | | gb = AI835847/gi = 5470060/ug = Mm 29897/len = 333/STRA = for | | | | | | |
| 8 | 96292_r_at | Cluster Incl AI835847 UI-M-AI0-aat-b-07-0-UI s1 Mus musculus cDNA, 3 end/clone = UI-M-AI0-aat-b-07-0-UI/clone_end = 3/ | 1650.9 | P | 1339 | P | 1456.1 | P |
| | | gb = AI835847/gi = 5470060/ug = Mm 29897/len = 333/STRA = for | | | | | | |
| 8 | 96305_at | Cluster Incl AI845854.UI-M-AO1-aeg-e-10-0-UI s1 Mus musculus cDNA, 3 end/clone = UI-M-AO1-aeg-e-10-0-UI/ | 273.4 | P | 282.9 | P | 396.9 | P |
| | | clone_end = 3/gb = AI845854/gi = 5489760/ug = Mm 29916/len = 441/STRA = for | | | | | | |
| 8 | 96321_at | Cluster Incl AI846396: UI-M-AN1-afa-h-08-0-UI s1 Mus musculus cDNA, 3 end/clone = UI-M-AN1-afa-h-08-0-UI/ | 1271.9 | P | 1241.2 | P | 1476.3 | P |
| | | clone_end = 3/gb = AI846396/gi = 5490302/ug = Mm 29939/len = 380/STRA = for | | | | | | |
| 8 | 96329_at | Cluster Incl AI837793.UI-M-AK0-adl-e-09-0-UI s1 Mus musculus cDNA, 3 end/clone = UI-M-AK0-adl-e-09-0-UI/ | 373.5 | P | 336.2 | P | 480.6 | P |
| | | clone_end = 3/gb = AI837793/gi = 5472006/ug = Mm 29965/len = 480/STRA = for | | | | | | |
| 8 | 96341_at | Cluster Incl AI849280 UI-M-AJ1-agz-h-05-0-UI.s1 Mus musculus cDNA, 3 end/clone = UI-M-AJ1-agz-h-05-0-UI/ | 363.2 | P | 199 | P | 256.9 | P |
| | | clone_end = 3/gb = AI849280/gi = 5493186/ug = Mm 29989/len = 520/STRA = for | | | | | | |
| 8 | 96342_at | Cluster Incl AI846320: UI-M-AN1 -afa-a-04-0-U1.s1 Mus musculus cDNA, 3 end/clone = UI-M-AN1-afa-a-04-0-UI/ | 346.6 | P | 321.5 | P | 378.6 | P |
| | | clone_end = 3/gb = AI846320/gi = 5490226/ug = Mm.29990/len = 281/STRA = for | | | | | | |
| 8 | 96360_at | Cluster Incl AW125498: UI-M-BH2.2-aqm-d-11-0-UI.s1 Mus musculus cDNA, 3 end/clone = UI-M-BH2.2-aqm-d-11-0-UI/ | 1943.2 | P | 2134.5 | P | 1912.8 | P |
| | | clone_end = 3/gb = AW125498/gi = 6101028/ug = Mm 30016/len = 389/STRA = for | | | | | | |
| 8 | 96855_at | Cluster Incl AI839946: UI-M-AHO-acs-a-04-0-UI.s1 Mus musculus cDNA, 3 end/clone = UI-M-AH0-acs-a-04-0-UI/ | 627.4 | P | 794.4 | P | 755.1 | P |
| | | clone_end = 3/gb = AI839946/gi = 5474159/ug = Mm 30043/len = 255/STRA = for | | | | | | |
| 8 | 96883_at | Cluster Incl U70733 Mus musculus translation initiation factor eIF3-p44 mRNA, complete cds/cds = (17,979)/gb = U70733/ | 1210.6 | P | 806.7 | P | 1318.9 | P |
| | | gi = 4097872/ug = Mm 30080/len = 1025/STRA = for | | | | | | |
| 8 | 96952_at | Cluster Incl AW121552 UI-M-BH2 2-aop-f-02-0-UI s1 Mus musculus cDNA, 3 end/clone = UI-M-BH2 2-aop-f-02-0-UI/ | 1831.5 | P | 1773.3 | P | 2341 | P |
| | | clone_end = 3/gb = AW121552/gi = 6096885/ug = Mm 30210/len = 521/STRA = for | | | | | | |
| 8 | 97443_at | Cluster Incl AI850850 UI-M-BH0-ajt-c-05-0-UI s1 Mus musculus cDNA, 3 end/clone = UI-M-BH0-ajt-c-05-0-UI/clone_end = 3/ | 1298.5 | P | 1276.7 | P | 1285.1 | P |
| | | gb = AI850850/gi = 5494756/ug = Mm 30240/len = 321/STRA = for | | | | | | |
| 8 | 97528_at | Cluster Incl AF022811 Mus musculus cornichon mRNA, complete cds/cds = (85,519)/gb = AF022811/gi = 3114215/ | 602.9 | P | 660.9 | P | 682.3 | P |
| | | ug = Mm 3261/len = 1388/STRA = for | | | | | | |
| 8 | 98104_at | Cluster Incl AI842889 UI-M-AN1-afb-g-01-0-UI s1 Mus musculus cDNA, 3 end/clone = UI-M-AN1-alb-g-01-0-UI/ | 720.7 | P | 700.2 | P | 870.4 | P |
| | | clone_end = 3/gb = AI842889/gi = 5477102/ug = Mm 3479/len = 304/STRA = for | | | | | | |
| 8 | 98545_at | Cluster Incl X78683 B-cell receptor-associated protein 37/cds = (107,1003)/gb = X78683/gi = 541733/ug = Mm 36241/ | 1453.4 | P | 1494.4 | P | 1822.6 | P |
| | | len = 1287/STRA = for | | | | | | |
| 8 | 98575_at | Cluster Incl X13135 Fatty acid synthase/cds = (0,2516)/gb = X13135/gi = 50947/ug = Mm 3760/len = 3295/STRA = for | 487.8 | P | 957.7 | P | 890.3 | P |
| 8 | 98618_at | Cluster Incl AA624336 vn01g09.r1 Mus musculus cDNA, 5 end/clone = IMAGE-1006528/clone_end = 5/gb = AA624336/ | 415.7 | P | 355 | P | 447.9 | P |
| | | gi = 2528212/ug = Mm 3856/len = 474/STRA = for | | | | | | |
| 8 | 98631_g_at | Cluster Incl AW106745 um33b04 y1 Mus musculus cDNA, 5 end/clone = IMAGE-2236303/clone_end = 5/gb = AW106745/ | 241.2 | P | 286.4 | P | 314.3 | P |
| | | gi = 6077545/ug = Mm 38792/len = 600/STRA = for | | | | | | |
| 8 | 99104_at | Cluster Incl U49915.Mus musculus adipocyte specific protein adipoQ (adipoQ) mRNA, complete cds/cds = (16,759)/ | 805.1 | P | 1625.2 | P | 2002.7 | P |
| | | gb = U49915/gi = 1399497/ug = Mm 3969/len = 1152/STRA = for | | | | | | |
| 8 | 99108_s_at | Cluster Incl U15012 Growth hormone receptor/cds = (0,430)/gb = U15012/gi = 666863/ug = Mm 3986/len = 513/STRA = for | 111.5 | P | 155.4 | P | 278.3 | P |
| 8 | 99140_at | Cluster Incl AW 124920 UI-M-BH2 1-apu-f-09-0-UI s1 Mus musculus cDNA, 3 end/clone = UI-M-BH2 1-apu-f-09-0-UI/ | 227.2 | P | 286.3 | P | 334.2 | P |
| | | clone_end = 3/gb = AW124920/gi = 6100450/ug = Mm 41390/len = 426/STRA = for | | | | | | |
| 8 | 99150_at | Cluster Incl AI844357.UI-M-AL1-ahp-a-10-0-UI.s1 Mus musculus cDNA, 3 end/clone = UI-M-AL1-ahp-a-10-0-UI/ | 483.1 | P | 417.5 | P | 508 | P |
| | | clone_end = 3/gb = AI844357/gi = 5488263/ug = Mm 41541/len = 413/STRA = for | | | | | | |
| 8 | 99564_at | Cluster Incl D87908 Mus musculus mRNA for nuclear protein np95, complete cds/cds = (148,2496)/gb = D87908/ | 449.9 | P | 279.5 | P | 335.2 | P |
| | | gi = 4220589/ug = Mm 42196/len = 3488/STRA = for | | | | | | |
| 8 | 99581_at | Cluster Incl U60001 Mus musculus protein kinase C inhibitor (mPKCI) mRNA, complete cds/cds = (22,402)/gb = U60001/ | 3142.3 | P | 2421.1 | P | 3262.2 | P |
| | | gi = 1519045/ug = Mm 425/len = 544/STRA = for | | | | | | |
| 8 | 99597_at | Cluster Incl AI841629 UI-M-AO0-acg-c-01-0-UI s1 Mus musculus cDNA, 3 end/clone = UI-M-AO0-acg-c-01-0-UI/ | 500.3 | P | 291 | P | 626 | P |
| | | clone_end = 3/gb = AI841629/gi = 5475842/ug = Mm 4284/len = 436/STRA = for | | | | | | |
| 8 | 100079_at | Cluster Incl AI845556 UI-M-AO1-aek-h-05-0-UI.s1 Mus musculus cDNA, 3 end/clone = UI-M-AO1-aek-h-05-0-UI/ | 1310.1 | P | 837.2 | P | 1474.3 | P |
| | | clone_end = 3/gb = AI845556/gi = 5489462/ug = Mm 4537/len = 428/STRA = for | | | | | | |
| 8 | 100081_at | Cluster Incl U27830 Mus musculus mSTI1 mRNA, complete cds/cds = (55,1686)/gb = U27830/gi = 881484/ug = Mm 4540/ | 400.7 | P | 448.7 | P | 513.6 | P |
| | | len = 2080/STRA = for | | | | | | |
| 8 | 100101_at | Cluster Incl L15447.Mouse small nuclear RNA (Rnu1a-1) mRNA, complete cds/cds = (99,962)/gb = L15447/gi = 349003/ | 267 | P | 541.5 | P | 628.2 | P |
| | | ug = Mm 4633/len = 1176/STRA = for | | | | | | |
| 8 | 100527_at | Cluster Incl AW124744 UI-M-BH2.1-aqa-d-10-0-UI s1 Mus musculus cDNA, 3 end/clone = UI-M-BH2 1-aqa-d-10-0-UI/ | 432.3 | P | 308.2 | P | 396.6 | P |
| | | clone_end = 3/gb = AW124744/gi = 6100274/ug = Mm.52/len = 485/STRA = for | | | | | | |
| 8 | 100575_at | Cluster Incl AI841645 UI-M-AO0-acg-d-06-0-UI s1 Mus musculus cDNA, 3 end/clone = UI-M-AO0-acg-d-06-0-UI/ | 270.3 | P | 127.3 | P | 280 | P |
| | | clone_end = 3/gb = AI841645/gi = 5475858/ug = Mm 5934/len = 256/STRA = for | | | | | | |
| 8 | 101016_at | Cluster Incl D87898 Mus musculus mRNA for ARF1, complete cds/cds = (78,623)/gb = D87898/gi = 1565206/ug = Mm.6836/ | 4141.3 | P | 4066.5 | P | 4530.5 | P |
| | | len = 1032/STRA = for | | | | | | |
| 8 | 101045_at | Cluster Incl U96116: Mus musculus amyloid beta-peptide binding protein (ERAB) mRNA, complete cds/cds = (104,898)/ | 702.5 | P | 611.9 | P | 706.8 | P |
| | | gb = U96116/gi = 2078283/ug = Mm.6994/len = 1029/STRA = for | | | | | | |
| 8 | 101085_at | Cluster Incl AI854257 UI-M-BH0-ajs-b-02-0-UI s1 Mus musculus cDNA, 3 end/clone = UI-M-BH0-ajs-b-02-0-UI/ | 756.8 | P | 627.5 | P | 843.2 | P |
| | | clone_end = 3/gb = AI854257/gi = 5498163/ug = Mm.7328/len = 511/STRA = for | | | | | | |
| 8 | 101112_g_at | Cluster Incl AF014371: Mus musculus Rho family GTPase (ArhA) mRNA, complete cds/cds = (134,715)/gb = AF014371/ | 864.7 | P | 960.9 | P | 961.4 | P |
| | | gi = 3237319/ug = Mm.757/len = 815/STRA = for | | | | | | |
| 8 | 101510_at | Cluster Incl AB007136 Protease (prosome, macropain) 28 subunit, alpha/cds = (81,830)/gb = AB007136/gi = 3219281/ | 442 | P | 411.2 | P | 752.9 | P |
| | | ug = Mm 830/len = 977/STRA = for | | | | | | |
| 8 | 101525_at | Cluster Incl AI848871 UI-M-AJ1-ahb-f-06-0-UI.s1 Mus musculus cDNA 3 end/clone = UI-M-AJ1-ahb-f-06-0-UI/ | 865.1 | P | 852.5 | P | 825.6 | P |
| | | clone_end = 3/gb = AI848871/gi = 5492777/ug = Mm.8688/len = 353/STRA = for | | | | | | |
| 8 | 101558_s_at | Cluster Incl AB003304 Proteasome beta type subunit 5/cds = (7,801)/gb = AB003304/gi = 2897805/ug = Mm 8911/len = 874/ | 914.3 | P | 912.5 | P | 1260.8 | P |
| | | STRA = for | | | | | | |
| 8 | 101954_at | Cluster Incl U70494 Mus musculus histone H2A Z (H2A Z) mRNA, complete cds/cds = (95,481)/gb = U70494/gi = 1575712/ | 2843.9 | P | 2566.9 | P | 2684.9 | P |
| | | ug = Mm 916/len = 989/STRA = for | | | | | | |
| 8 | 101961_at | Cluster Incl U67327.Mus musculus WD40-repeat type I transmembrane protein A72.5 mRNA, complete cds/ | 1013.6 | P | 1067.1 | P | 1188 | P |
| | | cds = (291,1031)/gb = U67327/gi = 1762983/ug = Mm 927/len = 1336/STRA = for | | | | | | |
| 8 | 101992_at | Cluster Incl U13393: Mus musculus delta proteasome subunit mRNA, complete cds/cds = (69,677)/gb = U13393/gi = 984937/ | 765.3 | P | 517.3 | P | 1010.4 | P |
| | | ug = Mm 98/len = 721/STRA = for | | | | | | |
| 8 | 102047_at | Cluster Incl AF043326 Mus musculus N-myristoyltransferase 1 mRNA complete cds/cds = (22,1512)/gb = AF043326/ | 497.8 | P | 584.4 | P | 597.6 | P |
| | | gi = 3005066/ug = Mm 10265/len = 1548/STRA = for | | | | | | |
| 8 | 102821_s_at | Cluster Incl L32752 Mouse (clone M2) GTPase (Ran) mRNA, complete cds/cds = (37,687)/gb = L32752/gi = 727168/ | 2026.5 | P | 2118.6 | P | 2575.7 | P |
| | | ug = Mm 14426/len = 970/STRA = for | | | | | | |
| 8 | 103350_at | Cluster Incl M64641: Moloney leukemia virus 34/cds = (78,1043)/gb = M64641/gi = 199770/ug = Mm.18347/len = 1547/ | 776.9 | P | 844.1 | P | 927.8 | P |
| | | STRA = for | | | | | | |
| 8 | 103371_at | Cluster Incl AF100956: Mus musculus major histocompatibility locus class II region; Fas-binding protein Daxx (DAXX) gene, | 689.5 | P | 658.9 | P | 830.1 | P |
| | | partial cds; Bing1 (BING1), tapasin (tapasin), RalGDS-like factor (RLF), KE2 (KE2), BING4 (BING4), beta1, 3-galactosyl | | | | | | |
| | | transferase (be/STRA = rev | | | | | | |
| 8 | 103630_at | Cluster Incl AI844089 UI-M-AL1-ahi-h-12-0-UI s1 Mus musculus cDNA, 3 end/clone = UI-M-AL1-ahi-h-12-0-UI/ | 270.7 | P | 390.5 | P | 330.2 | P |
| | | clone_end = 3/gb = AI844089/gi = 5487995/ug = Mm.20290/len = 241/STRA = for | | | | | | |
| 8 | 103821_at | Cluster Incl AJ223087: Mus musculus mRNA for Cdc6-related protein/cds = (103,1872)/gb = AJ223087/gi = 4007644/ | 101.7 | P | 107.9 | P | 92.6 | P |
| | | ug = Mm 20912/len = 2589/STRA = for | | | | | | |
| 8 | 103910_at | Cluster Incl AJ249987: Mus musculus TAFII30 gene for mTAFII30 protein, exons 1-5/cds = (25,681)/gb = AJ249987/ | 597.3 | P | 446.1 | P | 626 | P |
| | | gi = 6119622/ug = Mm.21419/len = 759/STRA = for | | | | | | |
| 8 | 104057_at | Cluster Incl AW012807 uo03a03 y1 Mus musculus cDNA, 5 end/clone = IMAGE-2582284/clone_end = 5/gb = AW012807/ | 175 | P | 181.2 | P | 281.8 | P |
| | | gi = 5861585/ug = Mm 21535/len = 544/STRA = for | | | | | | |
| 8 | 104279_at | Cluster Incl AW125116 UI-M-BH2.1-apx-h-12-0-UI s1 Mus musculus cDNA, 3 end/clone = UI-M-BH2.1-apx-h-12-0-UI/ | 1200.1 | P | 1166.2 | P | 1642.3 | P |
| | | clone_end = 3/gb = AW125116/gi = 6100646/ug = Mm 22230/len = 442/STRA = for | | | | | | |
| 8 | 104371_at | Cluster Incl AF078752 Mus musculus diacylglycerol acyltransferase (Dgat) mRNA, complete cds/cds = (95,1591)/ | 209.1 | P | 317.3 | P | 371 | P |
| | | gb = AF078752/gi = 3859933/ug = Mm 22633/len = 1650/STRA = for | | | | | | |
| 8 | 104560_at | Cluster Incl AW125634: UI-M-BH2.2-aqj-h-11-0-UI s1 Mus musculus cDNA, 3 end/clone = UI-M-BH2.2-aqj-h-11-0-UI/ | 179.3 | P | 152.7 | P | 320.1 | P |
| | | clone_end = 3/gb = AW125634/gi = 6101164/ug = Mm 23827/len = 447/STRA = for | | | | | | |
| 8 | 104573_at | Cluster Incl AA921069 vy83h09.r1 Mus musculus cDNA, 5 end/clone = IMAGE-1312865/clone_end = 5/gb = AA921069/ | 392.5 | P | 266.1 | P | 402.8 | P |
| | | gi = 3067848/ug = Mm 23925/len = 263/STRA = for | | | | | | |
| 8 | 104633_at | Cluster Incl AW123921. UI-M-BH2.3-aqc-a-07-0-UI s1 Mus musculus cDNA, 3 end/clone = UI-M-BH2 3-aqc-a-07-0-UI/ | 315.8 | P | 345.7 | P | 290.4 | P |
| | | clone_end = 3/gb = AW123921/gi = 6099451/ug = Mm 24342/len = 236/STRA = for | | | | | | |
| 8 | 94327_at | Cluster Incl AW230209 up28c10 y1 Mus musculus cDNA, 5 end/clone = IMAGE-2655666/clone_end = 5/gb = AW230209/ | 389 | P | 335.9 | P | 458.2 | P |
| | | gi = 6559505/ug = Mm 24875/len = 259/STRA = for | | | | | | |
| 8 | 94340_at | Cluster Incl AW124224 UI-M-BH2.1-aph-d-08-0-UI s1 Mus musculus cDNA, 3 end/clone = UI-M-BH2.1-aph-d-08-0-UI/ | 147.6 | P | 263.6 | P | 366.2 | P |
| | | clone_end = 3/gb = AW124224/gi = 6099754/ug = Mm 25042/len = 383/STRA = for | | | | | | |
| 8 | 95631_at | Cluster Incl AF088911: Mus musculus protein phosphatase X (Ppx) mRNA, complete cds/cds = (139,1062)/gb = AF088911/ | 1368.3 | P | 1205.1 | P | 1323.8 | P |
| | | gi = 4028534/ug = Mm 28082/len = 1276/STRA = rev | | | | | | |
| 8 | 96122_at | Cluster Incl AW049373 UI-M-BH1-ane-b-11-0-UI s1 Mus musculus cDNA, 3 end/clone = UI-M-BH1-ane-b-11-0-UI/ | 131.7 | P | 204.3 | P | 288.9 | P |
| | | clone_end = 3/gb = AW049373/gi = 5909902/ug = Mm.28108/len = 580/STRA = for | | | | | | |
| 8 | 96237_at | Cluster Incl AI118905 uc15f04 r1 Mus musculus cDNA, 5 end/clone = IMAGE-1398079/clone_end = 5/gb = AI118905/ | −60.8 | P | 273.6 | P | 155.6 | P |
| | | gi = 3519229/ug = Mm 28800/len = 311/STRA = for | | | | | | |
| 8 | 96834_at | Cluster Incl AI843586 UI-M-AO1-aeh-f-08-0-UI s1 Mus musculus cDNA, 3 end/clone = UI-M-AO1-aeh-f-08-0-UI/ | 961.4 | P | 928.3 | P | 977.8 | P |
| | | clone_end = 3/gb = AI843586/gi = 5477764/ug = Mm 29426/len = 398/STRA = for | | | | | | |
| 8 | 97342_at | Cluster Incl AW060867 UI-M-BH1-amx-f-08-0-UI s1 Mus musculus cDNA, 3 end/clone = UI-M-BH1-amx-f-08-0-UI/ | 953.1 | P | 756.6 | P | 810.5 | P |
| | | clone_end = 3/gb = AW060867/gi = 6008618/ug = Mm 29599/len = 418/STRA = for | | | | | | |
| 8 | 98446_s_at | Cluster Incl U06834 Mus musculus Balb/c eph-related receptor protein tyrosine kinase mRNA, complete cds/ | 489.7 | P | 477.9 | P | 596.6 | P |
| | | cds = (102,3065)/gb = U06834/gi = 459172/ug = Mm 34533/len = 3733/STRA = rev | | | | | | |
| 8 | 98495_at | Cluster Incl AI846906.UI-M-AK1-aey-b-11-0-UI.s1 Mus musculus cDNA, 3 end/clone = UI-M-AK1-aey-b-11-0-UI/ | 297.2 | P | 279.5 | P | 342.8 | P |
| | | clone_end = 3/gb = AI846908/gi = 5490812/ug = Mm 3561/len = 336/STRA = for | | | | | | |
| 8 | 100007_at | Cluster Incl AI837573 UI-M-AL0-abs-g-06-0-UI.s1 Mus musculus cDNA, 3 end/clone = UI-M-AL0-abs-g-06-0-UI/ | 847.9 | P | 846.3 | P | 847.7 | P |
| | | clone_end = 3/gb-AI837573/gi = 5471786/ug = Mm 4532/len = 293/STRA = for | | | | | | |
| 8 | 100959_at | Cluster Incl X99921.M musculus mRNA for S100 calcium-binding protein A13/cds = (60,356)/gb = X99921/gi = 1694840/ | 1277.8 | P | 1063.7 | P | 1205.6 | P |
| | | ug = Mm.6523/len = 440/STRA = for | | | | | | |
| 8 | 100964_at | Cluster Incl AF035208 Mus musculus putative v-SNARE Vti1b mRNA, complete cds/cds = (11,709)/gb = AF035208/ | 796.1 | P | 721.9 | P | 838.6 | P |
| | | gi = 3213228/ug = Mm 6577/len = 909/STRA = for | | | | | | |
| 8 | 100970_at | Cluster Incl X65687: Thymoma viral proto-oncogene/cds = (283,1725)/gb = X65687/gi = 287806/ug = Mm 6845/len = 2626/ | 652.3 | P | 647.3 | P | 1030.4 | P |
| | | STRA = for | | | | | | |
| 8 | 100990_g_at | Cluster Incl AJ001373 Mus musculus bodenin gene/cds = (173,775)/gb = AJ001373/gi = 2647955/ug = Mm 6818/len = 954/ | 294.3 | P | 281.8 | P | 368.6 | P |
| | | STRA = for | | | | | | |
| 8 | 101449_at | Cluster Incl AW120841 UI-M-BH2.3-aoa-e-02-0-UI s1 Mus musculus cDNA, 3 end/clone = UI-M-BH2.3-aoa-e-02-0-UI/ | 168.9 | P | 121.8 | P | 151.3 | P |
| | | clone_end = 3/gb = AW120841/gi = 6096174/ug = Mm 794/len = 283/STRA = for | | | | | | |
| 8 | 101461_f_at | Cluster Incl U06944 Praja1/cds = (328,1602)/gb = U06944/gi = 2828789/ug = Mm 8211/len = 1978/STRA = for | 1456.6 | P | 1160 | P | 1433.9 | P |
| 8 | 101905_at | Cluster Incl AF037454: lichy/cds = (149,2713)/gb = AF037454/gi = 2827197/ug = Mm 8975/len = 5122/STRA = for | 1101.9 | P | 1097 | P | 1755.6 | P |
| 8 | 101931_at | Cluster Incl AF042731: Mus musculus phosphatidylserine synthase-1 mRNA, complete cds/cds = (93,1514)/gb = AF042731/ | 716.9 | P | 772 | P | 759.1 | P |
| | | gi = 2811227/ug = Mm 9440/len = 2455/STRA = for | | | | | | |
| 8 | 102235_at | Cluster Incl X13945 Lung carcinoma myc related oncogene 1/cds = (0,1106)/gb = X13945/gi = 53287/ug = Mm.1055/ | 359.9 | P | 303.6 | P | 468.3 | P |
| | | len = 3076/STRA = for | | | | | | |
| 8 | 94105_at | Cluster Incl L78075: Cell division cycle 42/cds = (0,575)/gb = L78075/gi = 1321598/ug = Mm 1022/len = 576/STRA = for | 1249 | P | 1315.4 | P | 1190.2 | P |
| 8 | 100345_f_at | Cluster Incl W65964: me10a08 r1 Mus musculus cDNA, 5 and/clone = IMAGE-387062/clone_end = 5/gb = W65964/ | 1584.1 | P | 1413.2 | P | 1821.5 | P |
| | | gi = 1373784/ug = Mm.88247/len = 404/STRA = rev | | | | | | |
| 8 | 97060_at | Cluster Incl AW215489 up04f11 x1 Mus musculus cDNA, 3 end/clone = IMAGE-2651085/clone_end = 3/gb = AW215489/ | 366.4 | P | 335 | P | 303.9 | P |
| | | gi = 6526165/ug = Mm.89290/len = 522/STRA = rev | | | | | | |
| 8 | 100733_at | Cluster Incl X70303 Proteasome (prosome, macropain) subunit, alpha type 2/cds = (77,781)/gb = X70303/gi = 897783/ | 1383.4 | P | 1670.6 | P | 2039.5 | P |
| | | ug = Mm 3428/len = 904/STRA = for | | | | | | |
| 8 | 93252_at | Cluster Incl X81816 M musculus BAP31 mRNA/cds = (2,739)/gb = X81816/gi = 1487985/ug = Mm 17/len = 1132/STRA = for | 562.5 | P | 657.3 | P | 657.4 | P |
| 8 | 93741_at | Cluster Incl AA693066: vr61b07.s1 Mus musculus cDNA, 5 end/clone = IMAGE-1125109/clone_end = 5/gb = AA693066/ | 206.7 | M | 185.9 | P | 229.9 | P |
| | | gi = 2694004/ug = Mm.21062/len = 379/STRA = for | | | | | | |
| 8 | 94237_at | Cluster Incl AI846708.UI-M-AN1-afi-g-08-0-UI s1 Mus musculus cDNA, 3 end/clone = UI-M-AN1-afi-g-08-o-UI | 336.2 | P | 352 | P | 462.5 | P |
| | | clone_end = 3/gb = AI846708/gi = 5490614/ug = Mm 22333/len = 342/STRA = for | | | | | | |
| 8 | 98921_at | Cluster Incl AW125612: UI-M-BH2.2-aqj-f-12-0-UI s1 Mus musculus cDNA, 3 end/clone = UI-M-BH2.2-aqj-f-12-0-UI/ | 289.6 | P | 343.5 | A | 471 | P |
| | | clone_end = 3/gb = AW125812/gi = 6101142/ug = Mm 28626/len = 449/STRA = for | | | | | | |
| 8 | 98936_at | Cluster Incl AI837395: UI-M-AP0-abm-d-04-0-UI s1 Mus musculus cDNA, 3 end/clone = UI-M-AP0-abm-d-04-0-UI/ | 132 | A | −173.8 | P | 423.9 | P |
| | | clone_end = 3/gb = AI837395/gi = 5471608/ug = Mm 28688/len = 328/STRA = for | | | | | | |
| 8 | 95052_at | Cluster Incl AI839150 UI-M-AO0-ach-e-07-0-UI s1 Mus musculus cDNA, 3 end/clone = UI-M-AO0-ach-e-07-0-UI/ | 247.8 | P | 225.8 | P | 307.6 | P |
| | | clone_end = 3/gb = AI839150/gi = 5473363/ug = Mm 29140/len = 462/STRA = for | | | | | | |
| 8 | 99671_at | Cluster Incl X04673 Adipsin/cds = (19,798)/gb = X04673/gi = 49883/ug = Mm4407/len = 867/STRA = for | −125.4 | A | 1746.1 | P | 1949.5 | P |
| 8 | 102016_at | Cluster Incl M61737: M. musculus adipocyte-specific mRNA, partial cds/cds = UNKNOWN/gb = M61737/gi = 201107/ | 89.6 | M | 171.4 | P | 122.8 | P |
| | | ug = Mm 10026/len = 1725/STRA = for | | | | | | |
| 8 | 102400_at | Cluster Incl AW125118 UI-M-BH2 1-apz-a-03-0-UI s1 Mus musculus cDNA, 3 end/clone = UI-M-BH2.1-apz-a-03-0-UI/ | 152.9 | P | 213.2 | P | 267.3 | P |
| | | clone_end = 3/gb = AW125118/gi = 6100648/ug = Mm 12441/len = 270/STRA = for | | | | | | |
| 8 | 103398_at | Cluster Incl AW123232: UI-M-BH2 1-apd-g-08-0-UI s1 Mus musculus cDNA, 3 end/clone = UI-M-BH2 1-apd-g-08-0-UI/ | 163 | A | 137.7 | P | 256 | P |
| | | clone_end = 3/gb = AW123232/gi = 6098727/ug = Mm 18714/len = 469/STRA = rev | | | | | | |
| 8 | 103646_at | Cluster Incl X85983 Carnitine acetyltransferase/cds = (125,2008)/gb = X85983/gi = 758631/ug = Mm 20396/len = 2596/ | 162.6 | P | 100.5 | P | 155.6 | P |
| | | STRA = for | | | | | | |
| 8 | 104381_at | Cluster Incl AF085745 Mus musculus nuclear orphan receptor LXR-alpha mRNA, complete cds/cds = (84,1421)/ | 38.7 | A | 30.7 | P | 62.1 | P |
| | | gb = AF085745/gi = 4322470/ug = Mm 22690/len = 1665/STRA = rev | | | | | | |
| 8 | 100900_at | Cluster Incl AW209053: un99c08 x1 Mus musculus cDNA, 3 end/clone = IMAGE-2582030/clone_end = 3/gb = AW209053/ | 238.4 | P | 295.3 | P | 447 | P |
| | | gi = 6514993/ug = Mm 57203/len = 446/STRA = for | | | | | | |
| 8 | 101393_at | Cluster Incl AJ001633: Annexin III/cds = (200,1171)/gb = AJ001633/gi = 2437839/ug = Mm 7214/len = 1602/STRA = for | 29.9 | A | 72 | P | 70.9 | P |
| 8 | 104444_at | Cluster Incl AA689927 v163c12 r1 Mus musculus cDNA, 5 end/clone = IMAGE-1167766/clone_end = 5/gb = AA689927/ | 173.5 | P | 186.8 | P | 259.1 | P |
| | | gi = 2690863/ug = Mm 29321/len = 163/STRA = for | | | | | | |
| 8 | 96598_at | Cluster Incl AA122714 mr02b11 r1 Mus musculus cDNA, 5 end/clone = IMAGE-5296253/clone_end = 5/gb = AA122714/ | 6.1 | P | 46.7 | P | 29 | P |
| | | gi = 1681720/ug = Mm 309/len = 506/STRA = for | | | | | | |
| 8 | 93080_at | Cluster Incl AF069954 G protein gamma 3 linked gene/cds = (305,1537)/gb = AF069954/gi = 3885499/ug = Mm 15985/ | 499.8 | A | 526.1 | P | 508.8 | P |
| | | len = 1654/STRA = for | | | | | | |
| 8 | 93342_at | Cluster Incl AI852665: UI-M-BH0-aji-b-02-0-UI s1 Mus musculus cDNA, 3 end/clone = UI-M-BH0-aji-b-02-0-UI/clone_end = 3/ | 97.9 | A | 122.3 | P | 122.1 | P |
| | | gb = AI852665/gi = 5496571/ug = Mm 18739/len = 503/STRA = for | | | | | | |
| 8 | 94080_at | Cluster Incl AI835718: UI-M-AI0-aan-c-10-0-UI s1 Mus musculus cDNA, 3 end/clone = UI-M-AI0-aan-c-10-0-UI/ | 94 | A | 65 | P | 141.5 | P |
| | | clone_end = 3/gb = AI835718/gi = 5469931/ug = Mm 22149/len = 348/STRA = for | | | | | | |
| 8 | 94313_at | Cluster Incl X96767: M musculus mRNA for U1 anRNP-specific protein C/cds = (31,510)/gb = X96767/gi = 1360016/ | 395.7 | A | 349.6 | P | 407.5 | P |
| | | ug = Mm 2265/len = 738/STRA = for | | | | | | |
| 8 | 95427_at | Cluster Incl AI843650: UI-M-AO1-aen-h-06-0-UI s1 Mus musculus cDNA, 3 end/clone = UI-M-AO1-aen-h-06-0-UI/ | 149.8 | P | 279.1 | P | 250.4 | P |
| | | clone_end = 3/gb = AI843650/gi = 5477863/ug = Mm 24455/len = 449/STRA = for | | | | | | |
| 8 | 98538_at | Cluster Incl X81632 M musculus mRNA for e1 protein/cds = UNKNOWN/gb = X81632/gi = 563507/ug = Mm 35854/len = 3769/ | 228.3 | A | 111 | P | 353.7 | P |
| | | STRA = for | | | | | | |
| 8 | 101526_at | Cluster Incl X14759 Homeo box, msh-like 1/cds = (141,1112)/gb = X14759/gi = 51431/ug = Mm 870/len = 1810/STRA = for | 362.4 | A | 503.2 | P | 697.6 | P |
| 8 | 103853_at | Cluster Incl AI845664 UI-M-AQ1-ady-c-07-0-UI s1 Mus musculus cDNA, 3 end/clone = UI-M-AQ1-ady-c-07-0-UI/ | 59.2 | P | 75.7 | P | 94.8 | P |
| | | clone_end = 3/gb = AI845664/gi = 5489570/ug = Mm 21070/len = 184/STRA = for | | | | | | |
| 8 | 104120_at | Cluster Incl AW049205 UI-M-BH1-anb-a-02-0-UI s1 Mus musculus cDNA, 3 end/clone = UI-M-BH1-anb-a-02-0-UI/ | 700 | P | 582.9 | M | 794.1 | P |
| | | clone_end = 3/gb = AW049205/gi = 5909734/ug = Mm 21978/len = 412/STRA = for | | | | | | |
| 8 | 104287_at | Cluster Incl AI853807 UI-M-BH0-ajg-h-06-0-UI s1 Mus musculus cDNA, 3 end/clone = UI-M-BH0-ajg-h-06-0-UI/ | 86.2 | P | 359 | P | 423.2 | P |
| | | clone_end = 3/gb = AI853807/gi = 5497713/ug = Mm.22271/len = 445/STRA = for | | | | | | |
| 8 | 97934_at | Cluster Incl U73820 Mus musculus polypeptide GalNac transferase-T1 (ppGaNTase-T1) mRNA, complete cds/ | 75.6 | P | 59.8 | A | 157.8 | P |
| | | cds = (0,1679)/gb = U73820/gi = 2149048/ug = Mm.30249/len = 1680/STRA = for | | | | | | |
| 8 | 103506_f_at | Cluster Incl AW228162 up14b05 x1 Mus musculus cDNA, 3 end/clone = IMAGE-2654289/clone_end = 3/gb = AW228162/ | 84.7 | P | 100.8 | M | 61.9 | A |
| | | gi = 6557458/ug = Mm 2247/len = 414/STRA = for | | | | | | |
| 8 | 92805_s_at | Cluster Incl Y12577 ADP-ribosylation-like 4/cds = (243,845)/gb = Y12577/gi = 3288541/ug = Mm 12723/len = 1168/STRA = for | −117.6 | A | −181.4 | A | 132.6 | P |
| 8 | 95453_f_at | Cluster Incl AF087687 Mus musculus calcium-binding protein S100A1 mRNA, complete cds/cds = (110,394)/gb = AF087687/ | 152.9 | A | 186.9 | M | 223.8 | P |
| | | gi = 3746891/ug = Mm.24662/len = 506/STRA = for | | | | | | |
| 8 | 95480_at | Cluster Incl AI847163 UI-M-AO1-aep-d-12-0-UI s1 Mus musculus cDNA, 3 end/clone = UI-M-AO1-aep-d-12-0-UI/ | −24.3 | P | −107.3 | A | −38.1 | P |
| | | clone_end = 3/gb = AI847163/gi = 5491069/ug = Mm 24855/len = 148/STRA = for | | | | | | |
| 8 | 96110_at | Cluster Incl U31966: Carbonyl reductase/cds = (128,961)/gb = U31966/gi = 1049107/ug = Mm.26940/len = 1053/STRA = for | 267 | P | 322 | A | 322.7 | P |
| 8 | 96604_at | Cluster Incl AW125827 UI-M-BH2 2-aqk-b-10-0-UI s1 Mus musculus cDNA, 3 end/clone = UI-M-BH2 2-aqk-b-10-0-UI/ | 143.3 | P | 114.2 | A | 101.4 | P |
| | | clone_end = 3/gb = AW125827/gi = 6101357/ug = Mm 27039/len = 461/STRA = for | | | | | | |
| 8 | 98920_g_at | Cluster Incl AA867773: vx16d06.r1 Mus musculus cDNA, 5 end/clone = IMAGE-1284619/clone_end = 5/gb = AA867773/ | 161.5 | A | 182.8 | P | 243 | P |
| | | gi = 2963218/ug = Mm.28626/len = 385/STRA = for | | | | | | |
| 8 | 98588_at | Cluster Incl Z11774 Fumarylacetoacetate hydrolase/cds = (31,1290)/gb = Z11774/gi = 50972/ug = Mm 3798/len = 1398/ | 172.7 | A | 268.8 | A | 264.6 | P |
| | | STRA = rev | | | | | | |
| 8 | 102232_at | Cluster Incl AI850740 UI-M-BG1-ail-g-10-0-UI s1 Mus musculus cDNA, 3 end/clone = UI-M-BG1-ail-g-10-0-UI/ | 67.6 | P | 124.7 | A | 131.6 | A |
| | | clone_end = 3/gb = AI850740/gi = 5494646/ug = Mm 1040/len = 404/STRA = for | | | | | | |
| 8 | 103735_at | Cluster Incl M89800 Wingless-related MMTV integration site 6/cds = (0,1094)/gb = M89800/gi = 202407/ug = Mm 2438/ | 20.1 | A | 79.3 | P | 59.3 | P |
| | | len = 1435/STRA = for | | | | | | |
| 8 | 104695_at | Cluster Incl AI639864 vx72d06 x1 Mus musculus cDNA, 3 end/clone = IMAGE-1280747/clone_end = 3/gb = AI639864/ | 133.7 | A | 231.9 | P | 207.5 | A |
| | | gi = 4702973/ug = Mm.33622/len = 347/STRA = for | | | | | | |
| 8 | 94514_s_at | Cluster Incl AI853439 UI-M-BH0-ajy-d-04-0-UI s1 Mus musculus cDNA, 3 end/clone = UI-M-BH0-ajy-d-04-0-UI/ | −10.5 | P | 57.2 | P | −4.4 | A |
| | | clone_end = 3/gb = AI853439/gi = 5497345/ug = Mm 28981/len = 465/STRA = for | | | | | | |
| 8 | 99146_at | Cluster Incl AW124355 UI-M-BH2 1-apq-h-02-0-UI s1 Mus musculus cDNA, 3 end/clone = UI-M-BH2 1-apq-h-02-0-UI/ | 16.3 | A | 25.6 | A | 104.8 | A |
| | | clone_end = 3/gb = AW124355/gi = 6099885/ug = Mm 41462/len = 237/STRA = for | | | | | | |
| 8 | 99600_at | Cluster Incl AI838337: UI-M-AO0-aby-h-03-0-UI.s1 Mus musculus cDNA, 3 end/clone = UI-M-AO0-aby-h-03-0-UI/ | 183.3 | P | 181.9 | A | 345.2 | P |
| | | clone_end = 3/gb = AI838337/gi = 5472550/ug = Mm.42855/len = 414/STRA = for | | | | | | |
| 8 | 104132_at | Cluster Incl AW047276: UI-M-BH1-amh-c-02-0-UI.s1 Mus musculus cDNA, 3 end/clone = UI-M-BH1-amh-c-02-0-UI/ | 18.6 | A | 76 | A | 88.1 | P |
| | | clone_end = 3/gb = AW047276/gi = 5907805/ug = Mm 22072/len = 602/STRA = for | | | | | | |
| 8 | 104318_at | Cluster Incl Z97018: Mus musculus mRNA for PEX5 protein/cds = (0,1919)/gb = Z97018/gi = 2222731/ug = Mm.22418/ | 42.5 | A | 53.3 | A | 150.5 | P |
| | | len = 1986/STRA = for | | | | | | |
| 8 | 94956_at | Cluster Incl AF021031 DiGeorge syndrome chromosome region 6/cds = (0,584)/gb = AF021031/gi = 2582382/ug = Mm.27155/ | 43.1 | P | −11.7 | A | 111.2 | A |
| | | len = 996/STRA = for | | | | | | |
| 8 | 99988_at | Cluster Incl AW122115 UI-M-BH2 2-aor-b-02-0-UI s1 Mus musculus cDNA, 3 end/clone = UI-M-BH2 2-aor-b-02-0-UI/ | −27.8 | P | −15.7 | A | 156.4 | P |
| | | clone_end = 3/gb = AW122115/gi = 6097532/ug = Mm.4471/len = 403/STRA = for | | | | | | |
| 8 | 101728_at | Cluster Incl S46665 C5a anaphylatoxin receptor = peptidergic G-protein-coupled receptor [mice, BALB/C, Genomic, 1114 nt | 706.3 | A | 529.3 | A | 881.3 | P |
| | | 2 segments]/cds = (0,1055)/gb = S46665/gi = 1680365/ug = Mm.57044/len = 1056/STRA = for | | | | | | |
| 8 | 94020_at | Cluster Incl Y17808 Mus musculus mRNA for A6 related protein/cds = (17,1066)/gb = Y17808/gi = 4468631/ug = Mm 21852/ | 139.1 | P | 136.9 | P | 109 | A |
| | | len = 1435/STRA = for | | | | | | |
| 8 | 96020_at | Cluster Incl M22531: Mouse complement C1q B chain mRNA, complete cds/cds = (156,917)/gb = M22531/gi = 192266/ | −100.8 | A | −76.2 | A | −68.8 | A |
| | | ug = Mm.2570/len = 1059/STRA = for | | | | | | |
| 8 | 96071_at | Cluster Incl AI835709 UI-M-AI0-aan-b-12-0-UI.s1 Mus musculus cDNA, 3 end/clone = UI-M-AI0-aan-b-12-0-UI/ | 384.4 | P | 741.3 | P | 846.1 | P |
| | | clone_end = 3/gb = AI835709/gi = 5469922/ug = Mm 265/len = 395/STRA = for | | | | | | |
| 8 | 97252_at | Cluster Incl AW120926 UI-M-BH2.3-aob-f-08-0-UI s1 Mus musculus cDNA, 3 end/clone = UI-M-BH2 3-aob-f-08-0-UI/ | −75.8 | M | −72.8 | M | 5.2 | A |
| | | clone_end = 3/gb = AW120926/gi = 6096259/ug = Mm 27871/len = 171/STRA = for | | | | | | |
| 8 | 95738_at | Cluster Incl AW124889 UI-M-BH2.1-apu-c-11-0-UI s1 Mus musculus cDNA, 3 end/clone = UI-M-BH2 1-apu-c-11-0-UI/ | 107.2 | A | 46.6 | A | 94.3 | P |
| | | clone_end = 3/gb = AW124889/gi = 6100419/ug = Mm 29751/len = 386/STRA = for | | | | | | |
| 8 | 98606_s_at | Cluster Incl X69656 M musculus (clone S5) WRS mRNA for tryptophan-tRNA ligase/cds = (0,1427)/gb = X69656/gi = 55435/ | 143.4 | A | 140.5 | A | 118.4 | M |
| | | ug = Mm 38433/len = 1428/STRA = for | | | | | | |
| 8 | 101027_s_at | Cluster Incl AF069051 Mus musculus pituitary tumor transfroming gene protein (PTTG) mRNA, complete cds/ | 145 | M | 219.1 | A | 363.6 | M |
| | | cds = (303,893)/gb = AF069051/gi = 3702843/ug = Mm 6856/len = 945/STRA = for | | | | | | |
| 8 | 103643_at | Cluster Incl AI844784 UI-M-AL1-ahq-g-12-0-UI s1 Mus musculus cDNA, 3 end/clone = UI-M-AL1-ahq-g-12-0-UI/ | 90.7 | A | 184.4 | A | 129.4 | P |
| | | clone_end = 3/gb = AI844784/gi = 5488690/ug = Mm 20387/len = 264/STRA = rev | | | | | | |
| 8 | 104354_at | Cluster Incl X06368 Colony stimulating factor 1 receptor/cds = (75,3005)/gb = X06368/gi = 50980/ug = Mm.22574/len = 3665/ | 45 | A | 170.3 | A | 172 | A |
| | | STRA = for | | | | | | |
| 8 | 104719_at | Cluster Incl AI182203 uc68a04 r1 Mus musculus cDNA, 5 end/clone = IMAGE-1430766/clone_end = 5/gb = AI182203/ | −51.2 | A | 8 | A | 170.6 | P |
| | | gi = 3732841/ug = Mm 24510/len = 517/STRA = for | | | | | | |
| 8 | 92182_at | Cluster Incl AI836877 UI-M-AP0-abg-c-08-0-UI s2 Mus musculus cDNA, 3 end/clone = UI-M-AP0-abg-c-08-0-UI/ | 69.6 | P | 74.8 | A | 136.9 | P |
| | | clone_end = 3/gb = AI836877/gi = 5471090/ug = Mm 33851/len = 395/STRA = for | | | | | | |
| 8 | 93856_at | Cluster Incl M55512 Wilms tumor homolog/cds = UNKNOWN/gb = M55512/gi = 202413/ug = Mm 87561/len = 3089/ | 130 | P | 100.2 | A | 225.8 | A |
| | | STRA = for | | | | | | |
| 8 | 94512_f_at | Cluster Incl AI843210 UI-M-AK1-aet-f-05-0-UI s1 Mus musculus cDNA, 3 end/clone = UI-M-AK1-aet-f-05-0-UI/ | 141.6 | A | 116.9 | A | 181.4 | A |
| | | clone_end = 3/gb = AI843210/gi = 5477423/ug = Mm 28981/len = 418/STRA = for | | | | | | |
| 8 | 94515_at | Cluster Incl AW208628 uo62a08 x1 Mus musculus cDNA, 3 end/clone = IMAGE-2647094/clone_end = 3/gb = AW208628/ | −38.8 | A | −20.4 | A | 29.3 | A |
| | | gi = 6514568/ug = Mm.28986/len = 601/STRA = for | | | | | | |
| 8 | 94561_at | Clustar Incl AI836140 UI-M-AQ0-aai-b-09-0-UI s2 Mus musculus cDNA, 3 end/clone = UI-M-AQ0-aai-b-09-0-UI/ | 342.5 | A | 418.1 | P | 642.8 | A |
| | | clone_end = 3/gb = AI836140/gi = 5470353/ug = Mm 29107/len = 498/STRA = for | | | | | | |
| 8 | 97531_at | Cluster Incl X95280 Mus musculus mRNA for GOS2-like protein/cds = (132,443)/gb = X95280/gi = 1213012/ug = Mm 3283/ | −120.5 | A | −45.4 | A | 95.1 | A |
| | | len = 752/STRA = for | | | | | | |
| 8 | 101062_at | Cluster Incl AF054670: Heme oxygenase (decycling) 2/cds = (98,1045)/gb = AF054670/gi = 2984775/ug = Mm 710/len = 1192/ | 6.3 | M | −53.2 | A | 40.4 | P |
| | | STRA = for | | | | | | |
| 8 | 102366_at | Cluster Incl AA718169 yu54h05 r1 Mus musculus cDNA, 5 end/clone = IMAGE-1195257/clone_end = 5/gb = AA718169/ | −30.9 | A | 273.7 | A | 239.1 | A |
| | | gi = 2730443/ug = Mm 1181/len = 290/STRA = for | | | | | | |
| 8 | 101842_g_at | Cluster Incl AW049716 UI-M-BH1-ani-e-11-0-UI s1 Mus musculus cDNA, 3 end/clone = UI-M-BH1-ani-e-11-0-UI/ | 56.3 | A | 158.9 | P | 134.7 | P |
| | | clone_end = 3/gb = AW049716/gi = 5910245/ug = Mm 8534/len = 524/STRA = for | | | | | | |
| 8 | 93613_at | Cluster Incl AB008192 Mus musculus mRNA for mSmad3, complete cds/cds = (26,1303)/gb = AB008192/gi = 4630836/ | 37.2 | P | 14.1 | P | 39.5 | A |
| | | ug = Mm 7320/len = 1428/STRA = for | | | | | | |
| 9 | 92611_at | Cluster Incl U18773 Mus musculus GPI-anchored protein p137 mRNA, partial cds/cds = (0,550)/gb = U18773/gi = 862634/ | 707.8 | P | 932.3 | P | 947.6 | P |
| | | ug = Mm.1098/len = 2707/STRA = for | | | | | | |
| 9 | 92623_at | Cluster Incl AI847171: UI-M-AO1-aep-e-08-0-UI s1 Mus musculus cDNA, 3 end/clone = UI-M-AO1-aep-e-08-0-UI/ | 1612 | P | 1361.3 | P | 1442.5 | P |
| | | clone_end = 3/gb = AI847171/gi = 5491077/ug = Mm 11204/len = 208/STRA = for | | | | | | |
| 9 | 92639_at | Cluster Incl U80932 Senne/threonine kinase 6/cds = (48,1235)/gb = U80932/gi = 1763646/ug = Mm.11738/len = 1643/ | 139.4 | P | 66.4 | P | 177.6 | P |
| | | STRA = for | | | | | | |
| 9 | 92644_s_at | Cluster Incl M12848 Mouse myb proto-oncogene mRNA encoding 71 kd myb protein, complete cds/cds = (161,2071)/ | 69.1 | P | 70.4 | P | 87.3 | P |
| | | gb = M12848/gi = 199934/ug = Mm 1202/len = 3310/STRA = for | | | | | | |
| 9 | 92647_at | Cluster Incl U35141: Mus musculus retinoblastoma-binding protein (mRbAp48) mRNA, complete cds/cds = (107,1492)/ | 824.5 | P | 789.9 | P | 708.8 | P |
| | | gb = U35141/gi = 1016274/ug = Mm.12145/len = 1961/STRA = for | | | | | | |
| 9 | 92790_at | Cluster Incl D55720 Karyopherin (importin) alpha 2/cds = (177,1766)/gb = D55720/gi = 893392/ug = Mm 12508/len = 2048/ | 322.7 | P | 362.2 | P | 327.9 | P |
| | | STRA = for | | | | | | |
| 9 | 92809_r_at | Cluster Incl X17069 Mouse mRNA for protein with homology to transition protein 2 (TP2)/cds = (123,1358)/gb = X17069/ | 1078 | P | 863.1 | P | 866.8 | P |
| | | gi = 54875/ug = Mm 12758/len = 2026/STRA = for | | | | | | |
| 9 | 92826_at | Cluster Incl Y17852 Mus musculus mRNA for ganglioside-induced differentiation associated protein 3/cds = (0,385)/ | 296.3 | P | 350.1 | P | 247.7 | P |
| | | gb = Y17852/gi = 3378457/ug = Mm 12962/len = 651/STRA = for | | | | | | |
| 9 | 92829_at | Cluster Incl U09659 Mus musculus chaperonin 10 mRNA, complete cds/cds = (78,386)/gb = U09659/gi = 495205/ | 1236.1 | P | 1279.1 | P | 1351.4 | P |
| | | ug = Mm.12970/len = 462/STRA = for | | | | | | |
| 9 | 93013_at | Cluster Incl AF077861 Inhibitor of DNA binding 2/cds = (0,404)/gb = AF077861/gi = 3420740/ug = Mm.1466/len = 405/ | 583.8 | P | 443.8 | P | 451.8 | P |
| | | STRA = for | | | | | | |
| 9 | 93041_at | Cluster Incl D26089: Mini chromosome maintenance deficient 4 homolog (S. cerevisiae)/cds = (126,2714)/gb = D26089/ | 228.9 | P | 281.4 | P | 254.4 | P |
| | | gi = 940405/ug = Mm.1500/len = 3221/STRA = for | | | | | | |
| 9 | 93051_at | Cluster Incl Z37107: M. musculus mRNA for epoxide hydrolase/cds = (17,1681)/gb = Z37107/gi = 563509/ug = Mm.15295/ | 116.8 | P | 120.8 | P | 102.7 | P |
| | | len = 1990/STRA = for | | | | | | |
| 9 | 93069_at | Cluster Incl U62483 Mus musculus ubiquitin conjugating enzyme (ubc4) mRNA, complete cds/cds = (386,829)/gb = U62483/ | 204.5 | P | 145.6 | P | 102.2 | P |
| | | gi = 1480741/ug = Mm 1570/len = 2410/STRA = for | | | | | | |
| 9 | 93095_at | Cluster Incl U00431: Mus musculus HMG-1 mRNA, complete cds/cds = (145,792)/gb = U00431/gi = 437101/ug = Mm 16421/ | 379 | P | 325.1 | P | 205.9 | P |
| | | len = 1308/STRA = for | | | | | | |
| 9 | 93117_at | Cluster Incl AF073993: Mus musculus heterogenous nuclear ribonucleoprotein A2/B1 (hnRNP A2/B1) mRNA, complete cds/ | 1684.7 | P | 2362.3 | P | 1242.1 | P |
| | | cds = (96,1121)/gb = AF073993/gi = 3329497/ug = Mm 16767/len = 1677/STRA = for | | | | | | |
| 9 | 93250_r_at | Cluster Incl X67668 High mobility group protein 2/cds = (135,752)/gb = X67668/gi = 51338/ug = Mm 1693/len = 847/ | 815.8 | P | 841.2 | P | 511.6 | P |
| | | STRA = for | | | | | | |
| 9 | 93512_f_at | Cluster Incl AW121801 UI-M-BH2 3-anx-b-02-0-UI s2 Mus musculus cDNA, 3 end/clone = UI-M-BH2 3-anx-b-02-0-UI/ | 373 | P | 451.3 | P | 374.3 | P |
| | | clone_end = 3/gb = AW121801/gi = 6097161/ug = Mm 19352/len = 406/STRA = for | | | | | | |
| 9 | 93519_s_at | Cluster Incl AI847056 UI-M-AP1-agn-a-10-0-UI s1 Mus musculus cDNA, 3 end/clone = UI-M-AP1-agn-a-10-0-UI/ | 992.8 | P | 937.9 | P | 895.4 | P |
| | | clone_end = 3/gb = AI847056/gi = 5490962/ug = Mm 196/len = 185/STRA = for | | | | | | |
| 9 | 93541_at | Cluster Incl Z68618 Transgelin/cds = (12,617)/gb = Z68618/gi = 1160197/ug = Mm 2006/len = 1016/STRA = for | 431.8 | P | 524.8 | P | 445 | P |
| 9 | 93562_at | Cluster Incl AI854366 UI-M-BG1-aic-e-10-0-UI s1 Mus musculus cDNA, 3 end/clone = UI-M-BG1-aic-e-10-0-UI/ | 1223 | P | 908.2 | P | 929.7 | P |
| | | clone_end = 3/gb = AI854366/gi = 5498272/ug = Mm 2033/len = 410/STRA = for | | | | | | |
| 9 | 93772_i_at | Cluster Incl AI835041 UI-M-AI1-afn-d-02-0-UI s1 Mus musculus cDNA, 3 end/clone = UI-M-AI1-afn-d-02-0-UI/clone_end = 3/ | 436.9 | P | 692.9 | P | 422.4 | P |
| | | gb = AI835041/gi = 5469254/ug = Mm 21203/len = 409/STRA = for | | | | | | |
| 9 | 93786_i_at | Cluster Incl AI837302 UI-M-AK0-ade-h-11-0-UI s1 Mus musculus cDNA, 3 end/clone = UI-M-AK0-ade-h-11-0-UI | 388.2 | P | 559.8 | P | 379.2 | P |
| | | clone_end = 3/gb = AI837302/gi = 5471515/ug = Mm 21356/len = 392/STRA = for | | | | | | |
| 9 | 93787_f_at | Cluster Incl AI837302: UI-M-AK0-ade-h-11-0-UI s1 Mus musculus cDNA, 3 end/clone = UI-M-AK0-ade-h-11-0-UI/ | 1564.8 | P | 1514.1 | P | 1266.2 | P |
| | | clone_end = 3/gb = AI837302/gi = 5471515/ug-Mm 21356/len = 392/STRA = for | | | | | | |
| 9 | 93805_at | Cluster Incl AW121164 UI-M-BH2.3-aoh-g-12-0-UI s1 Mus musculus cDNA, 3 end/clone = UI-M-BH2 3-aoh-g-12-0-UI/ | 439.6 | P | 307.7 | P | 437.8 | P |
| | | clone_end = 3/gb = AW121164/gi = 6096497/ug = Mm 21444/len = 423/STRA = for | | | | | | |
| 9 | 93820_at | Cluster Incl AF037371 Mus musculus cytochrome c oxidase subunit VIIa-L precursor (Cox7al) mRNA, nuclear gene | 1530.4 | P | 1286.8 | P | 1352.7 | P |
| | | encoding mitochondrial protein, complete cds/cds = (70,321)/gb = AF037371/gi = 2708817/ug = Mm.2151/len = 461/ | | | | | | |
| | | STRA = for | | | | | | |
| 9 | 93997_at | Cluster Incl AI853475 UI-M-BH0-ajy-h-08-0-UI s1 Mus musculus cDNA, 3 end/clone = UI-M-BH0-ajy-h-08-0-UI/ | 481.7 | P | 300 | P | 396.2 | P |
| | | clone_end = 3/gb = AI853475/gi = 5497381/ug-Mm 21761/len = 472/STRA = for | | | | | | |
| 9 | 93999_at | Cluster Incl AW060597 UI-M-BH1-anq-d-09-0-UI s1 Mus musculus cDNA, 3 end/clone = UI-M-BH1-anq-d-09-0-UI/ | 1116.2 | P | 1157.5 | P | 1253.2 | P |
| | | clone_end = 3/gb = AW060597/gi = 6008348/ug = Mm 21764/len = 224/STRA = for | | | | | | |
| 9 | 94030_at | Cluster Incl AI853431: UI-M-BH0-ajy-c-06-0-UI s1 Mus musculus cDNA, 3 end/clone = UI-M-BH0-ajy-c-06-0-UI/ | 156.3 | P | 224.1 | P | 170.2 | P |
| | | clone_end = 3/gb = AI853431/gi = 5497337/ug = Mm 21901/len = 250/STRA = for | | | | | | |
| 9 | 94073_at | Cluster Incl AI836182 UI-M-AQ0-aaj-h-11-0-UI s2 Mus musculus cDNA, 3 end/clone = UI-M-AQ0-aaj-h-11-0-UI/ | 412 | P | 421.9 | P | 510.5 | P |
| | | clone_end = 3/gb = AI836182/gi = 5470395/ug = Mm.22117/len = 542/STRA = for | | | | | | |
| 9 | 94228_at | Cluster Incl AW123788 UI-M-BH2.1-apm-d-07-0-UI s1 Mus musculus cDNA, 3 end/clone = UI-M-BH2 1-apm-d-07-0-UI/ | 167.9 | P | 356.5 | P | 183.3 | P |
| | | clone_end = 3/gb = AW123788/gi = 6099318/ug-Mm 22269/len = 174/STRA = rev | | | | | | |
| 9 | 94267_i_at | Cluster Incl AW124386 UI-M-BH2 1-ape-b-11-0-UI s1 Mus musculus cDNA, 3 end/clone = UI-M-BH2 1-ape-d-11-0-UI/ | 1041.4 | P | 790.2 | P | 932.8 | P |
| | | clone_end = 3/gb = AW124386/gi = 6099916/ug = Mm.22470/len = 121/STRA = for | | | | | | |
| 9 | 94268_f_at | Cluster Incl AW124386 UI-M-BH2 1-ape-b-11-0-UI s1 Mus musculus cDNA, 3 end/clone = UI-M-BH2 1-ape-b-11-0-UI/ | 1948.3 | P | 1532.3 | P | 1643.9 | P |
| | | clone_end = 3/gb = AW124386/gi = 6099916/ug = Mm 22470/len = 121/STRA = for | | | | | | |
| 9 | 94294_at | Cluster Incl X66032 M musculus mRNA for cyclin B2/cds = (101,1297)/gb = X66032/gi = 50612/ug = Mm 22592/len = 1528/ | 1020.2 | P | 761.7 | P | 589.8 | P |
| | | STRA = for | | | | | | |
| 9 | 94806_at | Cluster Incl AW125336 UI-M-BH2 1-apy-g-01-0-UI s1 Mus musculus cDNA, 3 end/clone = UI-M-BH2.1-apy-g-01-0-UI/ | 449 | P | 418.4 | P | 551.8 | P |
| | | clone_end = 3/gb = AW125336/gi = 6100866/ug = Mm 22676/len = 253/STRA = for | | | | | | |
| 9 | 94885_at | Cluster Incl AI840062 UI-M-AL0-abt-b-05-0-UI s1 Mus musculus cDNA, 3 end/clone = UI-M-AL0-abt-b-05-0-UI/ | 126.8 | P | 125.2 | P | 116.4 | P |
| | | clone_end = 3/gb = AI840062/gi = 5474275/ug = Mm 23889/len = 499/STRA = for | | | | | | |
| 9 | 95423_at | Cluster Incl Y00884 Mouse mRNA (3 4J) from concanavalin A-stimulated LB3 T-cells/cds = (22,1770)/gb = Y00884/ | 362.2 | P | 448.8 | P | 276.7 | P |
| | | gi = 49727/ug = Mm 2442/len = 1957/STRA = for | | | | | | |
| 9 | 95451_at | Cluster Incl AA855382: vw72a04.r1 Mus musculus cDNA, 5 end/clone = IMAGE-1260462/clone_end = 5/gb = AA855382/ | 286.7 | P | 318 | P | 338.3 | P |
| | | gi = 2942920/ug = Mm 24641/len = 314/STRA = for | | | | | | |
| 9 | 95479_at | Cluster Incl X95591 M. musculus mRNA for C1D protein/cds = (77,502)/gb = X95591/gi = 1185124/ug = Mm 2485/len = 1050/ | 99.1 | P | 189.6 | P | 125.3 | P |
| | | STRA = for | | | | | | |
| 9 | 96054_f_at | Cluster Incl Y17345 Mus musculus mRNA for protein-tyrosine-phosphatase IF1/cds = (0,476)/gb = Y17345/gi = 3294228/ | 368.6 | P | 351.4 | P | 419.1 | P |
| | | ug = Mm 26166/len = 477/STRA = for | | | | | | |
| 9 | 96658_at | Cluster Incl AI841906: UI-M-AO0-acd-e-10-0-UI s1 Mus musculus cDNA, 3 end/clone = UI-M-AO0-acd-e-10-0-UI/ | 467.8 | P | 336.3 | P | 341 | P |
| | | clone_end = 3/gb = AI841906/gi = 5476119/ug = Mm 27344/len = 417/STRA = for | | | | | | |
| 9 | 96676_at | Cluster Incl AW121875 UI-M-BH2.3-aoi-d-05-0-UI s1 Mus musculus cDNA, 3 end/clone = UI-M-BH2 3-aoi-d-05-0-UI/ | 401.8 | P | 284.9 | P | 481.1 | P |
| | | clone_end = 3/gb = AW121875/gi = 6097249/ug = Mm.27406/len = 438/STRA = for | | | | | | |
| 9 | 96693_at | Cluster Incl AI849453 UI-M-AJ1-ahc-h-03-0-UI s1 Mus musculus cDNA, 3 end/clone = UI-M-AJ1-ahc-h-03-0-UI/ | 534.4 | P | 584.4 | P | 477.9 | P |
| | | clone_end = 3/gb = AI849453/gi = 5493359/ug = Mm.27526/len = 291/STRA = for | | | | | | |
| 9 | 96707_at | Cluster Incl D10630: Zinc finger protein 38/cds = (105,1772)/gb = D10630/ug = Mm 2760/len = 2103/STRA = for | 237.9 | P | 260.4 | P | 226 | P |
| 9 | 96711_at | Cluster Incl AI849191 UI-M-AJ1-agy-g-09-0-UI s1 Mus musculus cDNA, 3 end/clone = UI-M-AJ1-agy-g-09-0-UI/ | 268.5 | P | 270.7 | P | 213 | P |
| | | clone_end = 3/gb = AI849191/gi = 5493097/ug = Mm.27635/len = 469/STRA = for | | | | | | |
| 9 | 97200_f_at | Cluster Incl X65704 M musculus mRNA for small nuclear ribonucleoprotein E/cds = (0,245)/gb = X65704/gi = 312004/ | 2442.7 | P | 2237.8 | P | 2209.2 | P |
| | | ug = Mm.27669/len = 387/STRA = for | | | | | | |
| 9 | 97201_s_at | Cluster Incl AA823381: vp39a01.r1 Mus musculus cDNA, 5 end/clone = IMAGE-1079016/clone_end = 5/gb = AA823381/ | 734.6 | P | 849.6 | P | 797.8 | P |
| | | gi = 2893249/ug = Mm 27677/len = 339/STRA = for | | | | | | |
| 9 | 97254_at | Cluster Incl AA690061: vr7Bd03.s1 Mus musculus cDNA, 5 end/clone = IMAGE-1134821/clone_end = 5/gb = AA690061/ | 361.5 | P | 471.2 | P | 351.3 | P |
| | | gi = 2690997/ug = Mm.27875/len = 331/STRA = for | | | | | | |
| 9 | 97296_at | Cluster Incl AW124918: UI-M-BH2.1-apu-f-07-0-UI.s1 Mus musculus cDNA, 3 end/clone = UI-M-BH2.1-apu-f-07-0-UI/ | 206.6 | P | 163.7 | P | 170.7 | P |
| | | clone _end = 3/gb = AW124918/gi = 6100448/ug = Mm.28039/len = 187/STRA = for | | | | | | |
| 9 | 97301_at | Cluster Incl AW124187: UI-M-BH2.1-aph-a-06-0-UI.s1 Mus musculus cDNA, 3 end/clone = UI-M-BH2.1-aph-a-06-0-UI/ | 1107.5 | P | 1406.1 | P | 1105.9 | P |
| | | clone_end = 3/gb = AW124187/gb = 6099717/ug = Mm.28045/len = 382/STRA = for | | | | | | |
| 9 | 97307_f_at | Cluster Incl AI842808: UI-M-AP1-agi-f-09-0-UI.s1 Mus musculus cDNA, 3 end/clone = UI-M-AP1-agi-f-09-0-UI/clone_end = 3/ | 2907 | P | 2772.5 | P | 2865.8 | P |
| | | gb = AI842808/gi = 5477021/ug = Mm.28058/len = 337/STRA = for | | | | | | |
| 9 | 97315_at | Cluster Incl AW121730: UI-M-BH2.3-aoe-b-02-0-UI.s1 Mus musculus cDNA, 3 end/clone = UI-M-BH2.3-aoe-b-02-0-UI/ | 357.6 | P | 400.5 | P | 316.4 | P |
| | | clone _end = 3/gb = AW121730/gi = 6097063/ug = Mm.28097/len = 372/STRA = for | | | | | | |
| 9 | 97317_at | Cluster Incl AW122933: UI-M-BH2.1-apa-h-09-0-UI.s1 Mus musculus cDNA, 3 end/clone = UI-M-BH2.1-apa-h-09-0-UI/ | 497 | P | 374.9 | P | 554.4 | P |
| | | clone_end = 3/gb = AW122933/gi = 6098463/ug = Mm.28107/len = 399/STRA = for | | | | | | |
| 9 | 97846_at | Cluster Incl AI848968: UI-M-AJ1-ahd-c-01-0-UI.s1 Mus musculus cDNA, 3 end/clone = UI-M-AJ1-ahd-c-01-0-UI/ | 395.4 | P | 423.7 | P | 405.2 | P |
| | | clone_end = 3/gb = AI848968/gb = 5492874/ug = Mm.28270/len = 391/STRA = for | | | | | | |
| 9 | 97900_at | Cluster Incl AI845714: UI-M-AQ1-ady-h-04-0-UI.s1 Mus musculus cDNA, 3 end/clone = UI-M-AQ1-ady-h-04-0-UI/ | 488.7 | P | 412.5 | P | 385.4 | P |
| | | clone_end = 3/gb = AI845714/gi = 5489620/ug = Mm.28438/len = 384/STRA = for | | | | | | |
| 9 | 98914_at | Cluster Incl AI853634: UI-M-BH0-ajp-h-08-0-UI.s1 Mus musculus cDNA, 3 end/clone = UI-M-BH0-ajp-h-08-0-UI/ | 460.2 | P | 512.7 | P | 369.4 | P |
| | | clone_end = 3/gb = AI853634/gi = 5497540/ug = Mm.28606/len = 374/STRA = for | | | | | | |
| 9 | 98915_at | Cluster Incl AI849082: UI-M-AH1-agw-e-11-0-UI.s1 Mus musculus cDNA, 3 end/clone = UI-M-AH1-agw-e-11-0-UI/ | 427 | P | 414 | P | 368.7 | P |
| | | clone_end = 3/gb = AI849082/gi = 5492988/ug = Mm.28614/len = 494/STRA = for | | | | | | |
| 9 | 94489_at | Cluster Incl U84411: Mus musculus protein tyrosine phosphatase (PRL-1) mRNA, complete cds/cds = (713,1234)/ | 1512.7 | P | 2075.7 | P | 1708.9 | P |
| | | gb = U84411/gi = 1814023/ug = Mm.28909/len = 2849/STRA = for | | | | | | |
| 9 | 94508_at | Cluster Incl AI853772: UI-M-BH0-ajg-e-02-0-UI.s1 Mus musculus cDNA, 3 end/clone = UI-M-BH0-ajg-e-02-0-UI/ | 186.6 | P | 169.5 | P | 128.2 | P |
| | | clone_end = 3/gb = AI853772/gi = 5497678/ug = Mm.28963/len = 394/STRA = for | | | | | | |
| 9 | 95045_at | Cluster Incl AI844469: UI-M-AJ1-ahe-d-03-0-UI.s1 Mus musculus cDNA, 3 end/clone = UI-M-AJ1-ahe-d-03-0-UI/ | 190.8 | P | 350.8 | P | 267.4 | P |
| | | clone_end = 3/gb = AI844489/gi = 5488375/ug = Mm.29122/len = 406/STRA = for | | | | | | |
| 9 | 95063_at | Cluster Incl AI606257: vo03b06 x1 Mus musculus cDNA, 3 end/clone = IMAGE-1040339/clone_end = 3/gb = AI606257/ | 198.4 | P | 232.8 | P | 208.4 | P |
| | | gi = 4615424/ug = Mm.29164/len = 366/STRA = for | | | | | | |
| 9 | 95069_at | Cluster Incl AA940430: vz48h05 r1 Mus musculus cDNA, 5 end/clone = IMAGE-1329753/clone_end = 5/gb = AA940430/ | 582 | P | 360.4 | P | 431.9 | P |
| | | gi = 3099974/ug = Mm.29187/len = 468/STRA = for | | | | | | |
| 9 | 95105_at | Cluster Incl AI847697: UI-M-AP1-agm-d-08-0-UI.s1 Mus musculus cDNA, 3 end/clone = UI-M-AP1-agm-d-08-0-UI/ | 1061.5 | P | 1101.1 | P | 863.4 | P |
| | | clone_end = 3/gb = AI847697/gi = 5491603/ug = Mm.29353/len = 441/STRA = for | | | | | | |
| 9 | 95109_at | Cluster Incl AW121447: UI-M-BH2.2-aon-d-07-0-UI.s1 Mus musculus cDNA, 3 end/clone = UI-M-BH2.2-aon-d-07-0-UI/ | 230.4 | P | 246 | P | 324.4 | P |
| | | clone_end = 3/gb = AW121447/gi = 6096780/ug = Mm.29363/len = 278/STRA = for | | | | | | |
| 9 | 95119_at | Cluster Incl AA866888: vx90e09 r1 Mus musculus cDNA, 5 end/clone = IMAGE-1282504/clone_end = 5/gb = AA866888/ | 101.7 | P | 125.7 | P | 142.7 | P |
| | | gi = 2962333/ug = Mm 29382/len = 420/STRA = for | | | | | | |
| 9 | 95673_s_at | Cluster Incl AW124113: UI-M-BH2.1-apr-b-09-0-UI.s1 Mus musculus cDNA, 3 end/clone = UI-M-BH2.1-apr-b-09-0-UI/ | 1653 | P | 1657.2 | P | 1619.5 | P |
| | | clone_end = 3/gb = AW124113/gi = 6099643/ug = Mm.29588/len = 265/STRA = for | | | | | | |
| 9 | 95699_f_at | Cluster Incl AI848094: UI-M-AP1-agk-d-05-0-UI.s1 Mus musculus cDNA, 3 end/clone = UI-M-AP1-agk-d-05-0-UI/ | 894.3 | P | 1084.3 | P | 858.1 | P |
| | | clone_end = 3/gb = AI848094/gi = 5492000/ug = Mm.29685/len = 399/STRA = for | | | | | | |
| 9 | 96865_at | Cluster Incl M60474: Mynstoylated alanine rich protein kinase C substrate/cds = (380,1309)/gb = M60474/gi = 199026/ | 1031.1 | P | 954.7 | P | 754.8 | P |
| | | ug = Mm.30059/len = 2597/STRA = for | | | | | | |
| 9 | 96891_at | Cluster Incl AI842771: UI-M-AP1-agi-c-05-0-UI.s1 Mus musculus cDNA, 3 end/clone = UI-M-AP1-agi-c-05-0-UI/ | 2083 | P | 2001 | P | 1807.7 | P |
| | | clone_end = 3/gb = AI842771/gi = 5476984/ug = Mm.30093/len = 354/STRA = for | | | | | | |
| 9 | 96899_at | Cluster Incl AW123802: UI-M-BH2.1-apm-e-09-0-UI.s1 Mus musculus cDNA, 3 end/clone = UI-M-BH2.1-apm-e-09-0-UI/ | 1132.9 | P | 980.3 | P | 1203.1 | P |
| | | clone_end = 3/gb = AW123802/gi = 6099332/ug = Mm.30113/len = 371/STRA = for | | | | | | |
| 9 | 96947_at | Cluster Incl AW046273: UI-M-BH1-ala-f-05-0-UI.s1 Mus musculus cDNA, 3 end/clone = UI-M-BH1-ala-f-05-0-UI/ | 955.8 | P | 697.9 | P | 976.5 | P |
| | | clone_end = 3/gb = AW046273/gi = 5906802/ug = Mm.30200/len = 374/STRA = for | | | | | | |
| 9 | 96951_at | Cluster Incl AI839795: UI-M-AP0-abf-a-02-0-UI.s2 Mus musculus cDNA, 3 end/clone = UI-M-AP0-abf-a-02-0-UI/ | 641.9 | P | 757.3 | P | 629.7 | P |
| | | clone_end = 3/gb = AI839795/gi = 5474048/ug = Mm.30206/len = 475/STRA = for | | | | | | |
| 9 | 97445_at | Cluster Incl AW122022: UI-M-BH2.2-aoo-a-09-0-UI.s1 Mus musculus cDNA, 3 end/clone = UI-M-BH2.2-aoo-a-09-0-UI/ | 245 | P | 312.3 | P | 263.8 | P |
| | | clone_end = 3/gb = AW122022/gi = 6097428/ug = Mm.30242/len = 507/STRA = for | | | | | | |
| 9 | 97468_at | Cluster Incl AB025409: Mus musculus mRNA for aid1334p, complete cds/cds = (78,317)/gb = AB025409/gi = 5931566/ | 582.5 | P | 424.7 | P | 509.2 | P |
| | | ug = Mm.3049/len = 757/STRA = for | | | | | | |
| 9 | 97474_f_at | Cluster Incl D90225: Pleiotrophin/cds = (214,720)/gb = D90225/gi = 220553/ug = Mm.3063/len = 1514/STRA = for | 137.5 | P | 121 | P | 54.3 | P |
| 9 | 97478_at | Cluster Incl AW046965: UI-M-BH1-alp-c-09-0-UI.s1 Mus musculus cDNA, 3 end/clone = UI-M-BH1-alp-c-09-0-UI/ | 218.5 | P | 277.8 | P | 250.2 | P |
| | | clone_end = 3/gb = Aw046965/gi = 5907494/ug = Mm.30718/len = 368/STRA = for | | | | | | |
| 9 | 97527_at | Cluster Incl AA681998: vr45a06 s1 Mus musculus cDNA, 5 end/clone = IMAGE-1123570/clone_end = 5/gb = AA681998/ | 431.4 | P | 273.6 | P | 273.9 | P |
| | | gi = 2664138/ug = Mm.326/len = 463/STRA = for | | | | | | |
| 9 | 97543_at | Cluster Incl D49382: Mus musculus Nedd5 mRNA for septin, complete cds/cds = (53,1138)/gb = D49382/gi = 4519167/ | 623.9 | P | 711.6 | P | 604.9 | P |
| | | ug = Mm.336/len = 3170/STRA = for | | | | | | |
| 9 | 97556_at | Cluster Incl AI843178: UI-M-AK1-aet-c-07-0-UI.s1 Mus musculus cDNA, 3 end/clone = UI-M-AK1-aet-c-07-0-UI/ | 508.3 | P | 435.4 | P | 310.9 | P |
| | | clone_end = 3/gb = AI843178/gi = 5477391/ug = Mm.33875/len = 452/STRA = for | | | | | | |
| 9 | 98038_at | Cluster Incl AF022465: Mus musculus high mobility group protein homolog HMG4 (Hmg4) mRNA, complete cds/ | 1284.2 | P | 1011.1 | P | 813.9 | P |
| | | cds = (27,829)/gb = AF022465/gi = 2738988/ug = Mm.340/len = 1502/STRA = for | | | | | | |
| 9 | 98072_f_at | Cluster Incl X77731: M. musculus mRNA for Deoxycytidine kinase/cds = (12,794)/gb = X77731/gi = 456676/ug = Mm.3446 | 62.9 | P | 59.7 | P | 49 | P |
| | | len = 2812/STRA = for | | | | | | |
| 9 | 98075_at | Cluster Incl AW121683: UI-M-BH2.3-aoc-e-07-0-UI.s1 Mus musculus cDNA, 3 end/clone = UI-M-BH2.3-aoc-e-07-0-UI/ | 236.2 | P | 252 | P | 254.7 | P |
| | | clone_end = 3/gb = AW121683/gi = 6097916/ug = Mm.34526/len = 543/STRA = for | | | | | | |
| 9 | 98128_at | Cluster Incl U77128: Mus musculus mitochondrial ATP synthase coupling factor 6 mRNA, nuclear gene encoding | 992.2 | P | 981.1 | P | 865.1 | P |
| | | mitochondrial protein, complete cds/cds = (7,333)/gb = U77128/gi = 1679742/ug = Mm.353/len = 425/STRA = for | | | | | | |
| 9 | 98550_at | Cluster Incl AI854006: UI-M-BH0-alv-h-10-0-UI.s1 Mus musculus cDNA, 3 end/clone = UI-M-BH0-alv-h-10-0-UI/ | 1026.7 | P | 1515 | P | 1333.2 | P |
| | | clone_end = 3/gb = AI854006/gi = 5497912/ug = Mm.36677/len = 407/STRA = for | | | | | | |
| 9 | 98573_r_at | Cluster Incl X58045: RAN binding protein 1/cds = (137,748)/gb = X56045/gi = 443997/ug = Mm 3752/len = 1044/STRA = for | 833.7 | P | 1014.1 | P | 806.4 | P |
| 9 | 98610_at | Cluster Incl AW047926 UI-M- BH1-aln-c-12-0-UI s1 Mus musculus cDNA, 3 end/clone = UI-M-BH1-aln-c-12-0-UI/ | 169.2 | P | 158.6 | P | 158.8 | P |
| | | clone_end = 3/gb = AW047926/gi = 5908455/ug = Mm 38460/len = 359/STRA = for | | | | | | |
| 9 | 98613_at | Cluster Incl AI849876 UI-M-AL1-aho-h-03-0-UI.s1 Mus musculus cDNA, 3 end/clone = UI-M-AL1-aho-h-03-0-UI/ | 187 | P | 205.2 | P | 108.2 | P |
| | | clone_end = 3/gb = AI849876/gi = 5493775/ug = Mm 38521/len = 416/STRA = for | | | | | | |
| 9 | 98615_at | Cluster Incl AW049570 UI-M-BH1-anf-h-02-0-UI s1 Mus musculus cDNA, 3 end/clone = UI-M-BH1-anf-h-02-0-UI/ | 598.4 | P | 665.1 | P | 420.3 | P |
| | | clone_end = 3/gb = AW049570/gi = 5910099/ug = Mm 38524/len = 232/STRA = for | | | | | | |
| 9 | 99594_at | Cluster Incl AI844598 UI-M-AJ1-ahh-c-08-0-UI s2 Mus musculus cDNA, 3 end/clone = UI-M-AJ1-ahh-c-08-0-UI/ | 1072.1 | P | 1069.1 | P | 1021.6 | P |
| | | clone_end = 3/gb = AI844598/gi = 5488509/ug = Mm 42826/len = 329/STRA = for | | | | | | |
| 9 | 99632_at | Cluster Incl U83902 Mus musculus mitotic checkpoint component Mad2 mRNA, complete cds/cds = (48,665)/gb = U83902/ | 241.6 | P | 164.6 | P | 143.5 | P |
| | | gi = 4099130/ug = Mm 43444/len = 707/STRA = for | | | | | | |
| 9 | 99645_at | Cluster Incl AW048484 UI-M-BH1-alj-d-10-0-UI.s1 Mus musculus cDNA, 3 end/clone = UI-M-BH1-adj-d-10-o-UI/ | 607.8 | P | 537.5 | P | 544.7 | P |
| | | clone_end = 3/gb = AW048484/gi = 5909018/ug = Mm 43640/len = 458/STRA = for | | | | | | |
| 9 | 100113_s_at | Cluster Incl D50367: Mus musculus mRNA for KAP3B, complete cds/cds = (193,2511)/gb = D50367/gi = 1526420/ | 200.8 | P | 125.8 | P | 155.1 | P |
| | | ug = Mm 4651/len = 3908/STRA = for | | | | | | |
| 9 | 100116_at | Cluster Incl AI22538 uc62107 r1 Mus musculus cDNA, 5 end/clone = IMAGE-1430245/clone_end = 5/gb = AI122538/ | 736.5 | P | 561.7 | P | 483.7 | P |
| | | gi = 3522662/ug = Mm.46673/len = 505/STRA = for | | | | | | |
| 9 | 100522_s_at | Cluster Incl U92454 Mus musculus WW domain binding protein 5 mRNA, partial cds/cds = (0,185)/gb = U92454/ | 2044.8 | P | 1615.2 | P | 1514.5 | P |
| | | gi = 1914850/ug = Mm 512/len = 647/STRA = for | | | | | | |
| 9 | 100523_r_at | Cluster Incl U92454 Mus musculus WW domain binding protein 5 mRNA, partial cds/cds = (0,185)/gb = U92454/ | 405.9 | P | 421.9 | P | 376.6 | P |
| | | gi = 1914850/ug = Mm.512/len = 647/STRA = for | | | | | | |
| 9 | 100588_at | Cluster Incl U60329: Protease (prosome, macropain) 28 subunit, beta/cds = (1,720)/gb = U60329/gi = 1698571/ug = Mm 626/ | 688.5 | P | 608.7 | P | 649.6 | P |
| | | len = 723/STRA = for | | | | | | |
| 9 | 101001_at | Cluster Incl AI647612: uk41h01 x1 Mus musculus cDNA, 3 end/clone = IMAGE-1971601/clone_end = 3/gb = AI647612/ | 752.7 | P | 776.7 | P | 809.9 | P |
| | | gi = 4726290/ug = Mm.6766/len = 524/STRA = for | | | | | | |
| 9 | 101002_at | Cluster Incl AF032128 Mus musculus antizyms inhibitor mRNA, complete cds/cds = (16,1362)/gb = AF032128/gi = 2645700/ | 286.5 | P | 383.7 | P | 243.5 | P |
| | | ug = Mm.6775/len = 1599/STRA = for | | | | | | |
| 9 | 101004_f_at | Cluster Incl X91658: Splicing factor, arginine/serine-rich 3 (SRp20)/cds = (116,490)/gb = X91658/gi = 2125862/ug = Mm 6787/ | 793.8 | P | 871.4 | P | 734 | P |
| | | len = 1895/STRA = for | | | | | | |
| 9 | 101056_at | Cluster Incl X60672 M musculus mRNA for radixin/cds = (165,19166)/gb = X60672/gi = 1033049/ug = Mm.7051/len = 4238/ | 786.7 | P | 916 | P | 934 | P |
| | | STRA = for | | | | | | |
| 9 | 101060_at | Cluster Incl M73329: Mouse phospholipase C-alpha (PCL-alpha) mRNA, complete cds/cds = (79,1593)/gb = M73329/ | 966.8 | P | 992 | P | 922.8 | P |
| | | gi = 200396/ug = Mm.709/len = 1900/STRA = for | | | | | | |
| 9 | 101064_at | Cluster Incl AF044334: Mus musculus plelotropic regulator 1 (PLRG1) mRNA, complete cds/cds = (0,1541)/gb = AF044334/ | 510 | P | 499.1 | P | 539.7 | P |
| | | gi = 2832297/ug = Mm.7138/len = 1542/STRA = for | | | | | | |
| 9 | 101065_at | Cluster Incl X57800: Proliferating cell nuclear antigen/cds = (144,929)/gb = X57800/gi = 53601/ug = Mm.7141/len = 1261/ | 1077.5 | P | 944.1 | P | 1020.7 | P |
| | | STRA = for | | | | | | |
| 9 | 101067_at | Cluster Incl AW124711: UI-M-BH2 1-aqa-a-12-0-UI.s1 Mus musculus cDNA, 3 end/clone = UI-M-BH2.1-aqa-a-12-0-UI/ | 674.8 | P | 456.9 | P | 563.9 | P |
| | | clone_end = 3/gb = AW124711/gi = 6100241/ug = Mm 719/len = 467/STRA = for | | | | | | |
| 9 | 101091_at | Cluster Incl AF033353 Ubiquitin-like 1/cds = (118,423)/gb = AF033353/gi = 2645736/ug = Mm.7353/len = 1187/STRA = for | 597.1 | P | 835 | P | 772 | P |
| 9 | 101506_at | Cluster Incl AW227345 up 11a09 y1 Mus musculus cDNA, 5 end/clone = IMAGE-2651704/clone_end = 5/gb = AW227345/ | 310.4 | P | 310.5 | P | 283.9 | P |
| | | gi = 6556710/ug = Mm 821/len = 447/STRA = for | | | | | | |
| 9 | 101509_at | Cluster Incl U96760 Mus musculus von Hippel-Lindau binding protein homolog (vbp-1) mRNA, complete cds/ | 427.5 | P | 602.4 | P | 469.7 | P |
| | | cds = (101,583)/gb = U96760/gi = 2738245/ug = Mm 8294/len = 1531/STRA = for | | | | | | |
| 9 | 101580_at | Cluster Incl AI851220 UI-M-BH0-ejx-g-09-0-UI s1 Mus musculus cDNA, 3 end/clone = UI-M-BH0-ajx-g-09-0-UI/ | 1563.2 | P | 1949.3 | P | 1492.5 | P |
| | | clone_end = 3/gb = AI851220/gi = 5495126/ug = Mm.90007/len = 344/STRA = for | | | | | | |
| 9 | 101958_f_at | Cluster Incl X72310: Transcription factor Dp 1/cds = (54,1286)/gb = X72310/gi = 395280/ug = Mm 925/len = 1700/STRA = for | 401.9 | P | 437.9 | P | 469.8 | P |
| 9 | 101959_r_at | Cluster Incl X72310 Transcription factor Dp 1/cds = (54,1286)/gb = X72310/gi = 395280/ug = Mm 925/len = 1700/STRA = for | 596.6 | P | 659.7 | P | 708.1 | P |
| 9 | 102001_at | Cluster Incl M14223 Mouse ribonucleotide reductase M2 subunit mRNA, complete cds/cds = (62,1234)/gb = M14223/ | 357.4 | P | 433.5 | P | 304 | P |
| 9 | 102409_at | Cluster Incl AW046963 UI-M-BH1-alp-c-c7-0-UI s1 Mus musculus cDNA, 3 end/clone = UI-M-BH1-alp-c-07-0-UI/ | 353.4 | P | 471.8 | P | 290.5 | P |
| | | clone_end = 3/gb = AW046963/gi = 5907492/ug = Mm 12539/len = 307/STRA = for | | | | | | |
| 9 | 102776_at | Cluster Incl AI55095 ud59b08 r1 Mus musculus cDNA, 5 end/clone = IMAGE-1450163/clone_end = 5/gb = AI55095/ | 118.7 | P | −2.5 | P | −84.3 | P |
| | | gi = 3683564/ug = Mm 13437/len = 120/STRA = for | | | | | | |
| 9 | 103036_at | Cluster incl M38700 Thyroid autoantigen 70 kDa/cds = (0,1778)/gb = M38700/gi = 198636/ug = Mm.16147/len = 1944/ | 76.4 | P | 78.4 | P | 66.2 | P |
| | | STRA = for | | | | | | |
| 9 | 103342_at | Cluster Incl U78103: Embryonic ectoderm development/cds = (453,1778)/gb = U76103/gi = 1710987/ug = Mm 18203/ | 177.6 | P | 216.2 | P | 182.2 | P |
| | | len = 2040/STRA = for | | | | | | |
| 9 | 103402_at | Cluster Incl AI848522: UI-M-AP1-agf-e-06-0-UI s2 Mus musculus cDNA, 3 end/clone = UI-M-AP1-agf-e-06-0-UI/ | 283.7 | P | 179.7 | P | 117.1 | P |
| | | clone_end = 3/gb = AI848522/gi = 5492428/ug = Mm.18761/len = 425/STRA = for | | | | | | |
| 9 | 103406_at | Cluster Incl AI853967 UI-M-BH0-aiv-e-02-0-UI s1 Mus musculus cDNA, 3 end/clone = UI-M-BH0-aiv-e-02-0-UI/ | 163.1 | P | 242 | P | 153.2 | P |
| | | clone_end = 3/gb = AI853967/gi = 5497873/ug = Mm 18798/len = 434/STRA = for | | | | | | |
| 9 | 103418_at | Cluster Incl AW122092: UI-M-BH2.2-aoo-g-10-0-UI s1 Mus musculus cDNA, 3 end/clone = UI-M-BH2 2-aoo-g-10-0-UI/ | 210.9 | P | 142.6 | P | 126.8 | P |
| | | clone_end = 3/gb = AW122092/gi = 6097499/ug = Mm.18876/len = 451/STRA = for | | | | | | |
| 9 | 103852_at | Cluster Incl AI848207 UI-M-AH1-agq-g-08-0-UI s1 Mus musculus cDNA, 3 end/clone = UI-M-AH1-agq-g-08-0-UI/ | 204 | P | 178.5 | P | 175.8 | P |
| | | clone_end = 3/gb = AI848207/gi = 5492118/ug = Mm 21064/len = 455/STRA = for | | | | | | |
| 9 | 103873_i_at | Cluster Incl AA684456: vm64c06.s1 Mus musculus cDNA, 5 end/clone = IMAGE-1003018/clone_end = 5/gb = AA684456/ | 194.6 | P | 154.8 | P | 111.1 | P |
| | | gi = 2671042/ug = Mm.21173/len = 217/STRA = for | | | | | | |
| 9 | 103876_at | Cluster Incl AA760339: vv75c06.r1 Mus musculus cDNA, 5 end/clone = IMAGE-1228234/clone_end = 5/gb = AA760339/ | 538.9 | P | 459.7 | P | 580.8 | P |
| | | gi = 2808133/ug = Mm.21181/len = 552/STRA = for | | | | | | |
| 9 | 103881_at | Cluster Incl AA691185: vt34b11.r1 Mus musculus cDNA, 5 end/clone = IMAGE-1184957/clone_end = 5/gb = AA691185/ | 286.1 | P | 192.6 | P | 221.6 | P |
| | | gi = 2692121/ug = Mm.212/len = 184/STRA = for | | | | | | |
| 9 | 103886_at | Cluster Incl AI850356: UI-M-BG1-aig-a-12-0-UI s1 Mus musculus cDNA, 3 end/clone = UI-M-BG1-aig-a-12-0-UI/ | 366.9 | P | 347.2 | P | 343.2 | P |
| | | clone_end = 3/gb = AI850356/gi = 5494262/ug = Mm.21251/len = 443/STRA = for | | | | | | |
| 9 | 104038_at | Cluster Incl AI843511 UI-M-AO1-aeh-b-11-0-UI a1 Mus musculus cDNA, 3 end/clone = UI-M-AO1-aeh-b-11-0-UI/ | 324.3 | P | 234.9 | P | 301.2 | P |
| | | clone_end = 3/gb = AI843511/gi = 5477724/ug = Mm.21451/len = 459/STRA = for | | | | | | |
| 9 | 104052_at | Cluster Incl AI840921: UI-M-AMO-adm-f-12-0-UI s1 Mus musculus cDNA, 3 end/clone = UI-M-AM0-adm-f-12-0-UI/ | 231.6 | P | 151.2 | P | 166.3 | P |
| | | clone_end = 3/gb = AI840921/gi = 5475134/ug = Mm.21520/len = 204/STRA = for | | | | | | |
| 9 | 104077_at | Cluster Incl AW121992: UI-M-BH2.3-aoj-f-11-0-UI s1 Mus musculus cDNA, 3 end/clone = UI-M-BH2.3-aoj-f-11-0-UI/ | 213.5 | P | 243.5 | P | 244.3 | P |
| | | clone_end = 3/gb = AW121992/gi = 6097398/ug = Mm 21705/len = 477/STRA = for | | | | | | |
| 9 | 104090_at | Cluster Incl AA657164: vr26f01 r1 Mus musculus cDNA, 5 end/clone = IMAGE-1121785/clone_end = 5/gb = AA657164/ | 197.1 | P | 147.5 | P | 174.5 | P |
| | | gi = 2593318/ug = Mm.21803/len = 376/STRA = for | | | | | | |
| 9 | 104126_at | Cluster Incl AI854864: UI-M-BHO-akb-e-01-0-UI a1 Mus musculus cDNA, 3 end/clone = UI-M-BHO-akb-e-01-0-UI/ | 260.2 | P | 253.7 | P | 213.9 | P |
| | | clone_end = 3/gb = AI854864/gi = 5498770/ug = Mm.22031/len = 197/STRA = for | | | | | | |
| 9 | 104134_at | Cluster Incl Y17851: Mus musculus mRNA for ganglioside-induced differentiation associated protein 2/cds = (0,223)/ | 110.6 | P | 157.4 | P | 112 | P |
| | | gb = Y17851/gi = 3378455/ug = Mm.22080/len = 197/STRA = for | | | | | | |
| 9 | 104305_at | Cluster Incl AW120679: UI-M-BH2.3-anz-c-02-0-UI s1 Mus musculus cDNA, 3 end/clone = UI-M-BH2.3-anz-c-02-0-UI/ | 273.5 | P | 287.7 | P | 201.3 | P |
| | | clone_end = 3/gb = AW120679/gi = 6095977/ug = Mm.22363/len = 454/STRA = for | | | | | | |
| 9 | 104378_at | Cluster Incl L4851: Paraoxonase 2/cds = (24,1088)/gb = L48514/gi = 1333637/ug = Mm.22581/len = 1179/STRA = for | 62.2 | P | 166.1 | P | 72.5 | P |
| 9 | 104527_at | Cluster Incl D13803: Rad51 homolog (S cerevisiae)/cds = (188,1207)/gb = D13803/gi = 303702/ug = Mm.231/len = 1408/ | 263.7 | P | 178 | P | 191.3 | P |
| | | STRA = for | | | | | | |
| 9 | 104544_at | Cluster Incl AA675604: vr73b04.s1 Mus musculus cDNA, 5 end/clone = IMAGE-1134319/clone_end = 5/gb = AA675604/ | 179.3 | P | 210.5 | P | 129.8 | P |
| | | gi = 2652841/ug = Mm.23690/len = 469/STRA = for | | | | | | |
| 9 | 104565_at | Cluster Incl AI847561: UI-M-AM1-afv-g-05-0-UI s1 Mus musculus cDNA, 3 end/clone = UI-M-AM1-afv-g-05-0-UI/ | 203.9 | P | 306.4 | P | 234.9 | P |
| | | clone_end = 3/gb = AI847561/gi = 5491467/ug = Mm.23861/len = 370/STRA = for | | | | | | |
| 9 | 104586_at | Cluster Incl AI842878: UI-M-AN1-afb-e-0-UI.s1 Mus musculus cDNA, 3 end/clone = UI-M-AN1-afb-e-12-0-UI/ | 112.7 | P | 209.6 | P | 135.5 | P |
| | | clone_end = 3/gb = AI842878/gi = 5477091/ug = Mm.23985/len = 162/STRA = for | | | | | | |
| 9 | 104634_at | Cluster Incl AA874589: vx78a06 r1 Mus musculus cDNA, 5 end/clone = IMAGE-1281298/clone_end = 5/gb = AA874589/ | 473.4 | P | 518.2 | P | 533.7 | P |
| | | gi = 2979278/ug = Mm.24345/len = 559/STRA = for | | | | | | |
| 9 | 104735_at | Cluster Incl AI842065: UI-M-AN1-afg-a-10-0-UI.s1 Mus musculus cDNA, 3 end/clone = UI-M-AN1-afg-a-10-0-UI/ | 243.8 | P | 138.9 | P | 210.6 | P |
| | | clone_end = 3/gb = AI842065/gi = 5476278/ug = Mm.24647/len = 444/STRA = for | | | | | | |
| 9 | 104740_at | Cluster Incl AW125318: UI-M-BH2.1-apy-a-04-0-UI.s1 Mus musculus cDNA, 3 end/clone = UI-M-BH2.1-apy-a-04-0-UI/ | 136.9 | P | 140.2 | P | 92.9 | P |
| | | clone_end = 3/gb = AW125318/gi = 6100848/ug = Mm.24671/len = 405/STRA = for | | | | | | |
| 9 | 104766_at | Cluster Incl AI851198: UI-M-BH0-ajx-e-09-0-UI.s1 Mus musculus cDNA, 3 end/clone = UI-M-BH0-ajx-e-09-0-UI/ | 798.3 | P | 671.7 | P | 576.8 | P |
| | | clone_end = 3/gb = AI851198/gi = 5495104/ug = Mm.24873/len = 193/STRA = rev | | | | | | |
| 9 | 94346_at | Cluster Incl AI853721: UI-M-BH0-ajq-e-02-0-UI.s1 Mus musculus cDNA, 3 end/clone = UI-M-BH0-ajq-e-02-0-UI/ | 141.8 | P | 118.6 | P | 97.1 | P |
| | | clone_end = 3/gb = AI853721/gi = 5497592/ug = Mm.25154/len = 203/STRA = for | | | | | | |
| 9 | 94966_at | Cluster Incl Z11911: Glucose-6-phosphate dehydrogenase X-linked/cds = (52,1599)/gb = Z11911/gi = 51113/ug = Mm.27210/ | 523.6 | P | 678.1 | P | 613.6 | P |
| | | len = 2322/STRA = for | | | | | | |
| 9 | 94967_at | Cluster Incl AI851365: UI-M-BH0-akh-e-08-0-UI.s1 Mus musculus cDNA, 3 end/clone = UI-M-BH0-akh-e-08-0-UI/ | 304.6 | P | 342.5 | P | 374.6 | P |
| | | clone_end = 3/gb = AI851365/gi = 5495271/ug = Mm.27216/len = 481/STRA = for | | | | | | |
| 9 | 94982_f_at | Cluster Incl AI852470: UI-M-BH0-alp-f-08-0-UI.s1 Mus musculus cDNA, 3 end/clone = UI-M-BH0-alp-f-08-0-UI/ | 172.7 | P | 104.7 | P | 145 | P |
| | | clone_end = 3/gb = AI852470/gi = 5496376/ug = Mm.27278/len = 422/STRA = for | | | | | | |
| 9 | 94989_at | Cluster Incl AI848984: UI-M-AJ1-ahd-d-06-0UI.s1 Mus musculus cDNA, 3 end/clone = UI-M-AJ1-ahd-d-06-0-UI/ | 609.8 | P | 927.3 | P | 701 | P |
| | | clone_end = 3/gb = AI848984/gi = 5492890/ug = Mm.27311/len = 476/STRA = for | | | | | | |
| 9 | 94998_at | Cluster Incl AI838452: UI-M-AJ0-aba-e-08-0-UI.s1 Mus musculus cDNA, 3 end/clone = UI-M-AJ0-aba-e-08-0-UI/ | 117.6 | P | 175.6 | P | 127.4 | P |
| | | clone_end = 3/gb = AI838452/gi = 5472665/ug = MM.27348/len = 466/STRA = for | | | | | | |
| 9 | 95003_at | Cluster Incl AA80275: vv99ho2.r1 Mus musculus cDNA, 5 end/clone = IMAGE-1230579/clone_end = 5/gb = AA880275/ | 375.9 | P | 446.2 | P | 273.5 | P |
| | | gi = 2989258/ug = Mm.27375/len = 382/STRA = for | | | | | | |
| 9 | 95018_r_at | Cluster Incl AA607053: vm95e01.r1 Mus musculus cDNA, 5 end/clone = IMAGE-1006008/clone_end = 5/gb = AA607053/ | 308.6 | P | 332.2 | P | 317.2 | P |
| | | gi = 2455946/ug = Mm.27454/len = 178/STRA = for | | | | | | |
| 9 | 95614_at | Cluster Incl AI852086: UI-M-BH0-aja-g-03-0-UI.s1 Mus musculus cDNA, 3 end/clone = UI-M-BH0-aja-g-03-0-UI/ | 306.8 | P | 413.1 | P | 385 | P |
| | | clone_end = 3/gb = AI852086/gi = 5495992/ug = Mm.28003/len = 439/STRA = for | | | | | | |
| 9 | 96158_at | Cluster Incl AW123477: UI-M-BH2.1-apj-g-12-0-UI.s1 Mus musculus cDNA, 3 end/clone = UI-M-BH2.1-apj-g-12-0-UI/ | 305.6 | P | 300.3 | P | 326 | P |
| | | clone_end = 3/gb = AW123477/gi = 6099007/ug = Mm.28332/len = 423/STRA = for | | | | | | |
| 9 | 96168_at | Cluster Incl AI591702: vq88c03.y1 Mus musculus cDNA, 5 end/clone = IMAGE-1109380/clone_end = 5/gb = AI591702/ | 382.9 | P | 181.7 | P | 54.9 | P |
| | | gi = 4800750/ug = Mm.28386/len = 266/STRA = for | | | | | | |
| 9 | 96743_at | Cluster Incl AA958560: ua14109.r1 Mus musculus cDNA, 5 end/clone = IMAGE-1346729/clone_end = 5/gb = AA958560/ | 368.6 | P | 456.1 | P | 365 | P |
| | | gi = 3124790/ug = Mm.28938/len = 292/STRA = for | | | | | | |
| 9 | 97327_at | Cluster Incl L26320: Flap structure specific endonuclease 1/cds = (103,1239)/gb = L26320/gi = 499648/ug = Mm.2952/ | 303.7 | P | 326.9 | P | 275.1 | P |
| | | len = 2033/STRA = for | | | | | | |
| 9 | 97329_at | Cluster Incl AW124061: UI-M-BH2.1-apn-f-03-0-UI.s1 Mus musculus cDNA, 3 end/clone = UI-M-BH2.1-apn-f-03-0-UI/ | 173.1 | P | 184.5 | P | 181.4 | P |
| | | clone_end = 3/gb = AW124061/gi = 6099591/ug = Mm.29529/len = 437/STRA = for | | | | | | |
| 9 | 97411_at | Cluster Incl L11316: Ect2 oncogene/cds = (530,2746)/gb = L11316/gi = 293331/ug = Mm 2995/len = 3920/STRA = for | 188.1 | P | 181.4 | P | 164.4 | P |
| 9 | 97412_at | Cluster Incl AI843157: UI-M-AK1-aet-a-08-0-UI.s1 Mus musculus cDNA, 3 end/clone = UI-M-AK1-aet-a-08-0-UI/ | 374.5 | P | 164.4 | P | 292.2 | P |
| | | clone_end = 3/gb = AI843157/gi = 5477370/ug = Mm 29952/len = 443/STRA = for | | | | | | |
| 9 | 97425_at | Cluster Incl AI840191: UI-M-AO0-acc-g-09-0-UI.s1 Mus musculus cDNA, 3 end/clone = UI-M-AO0-acc-g-09-0-UI/ | 258.1 | P | 424.6 | P | 278.2 | P |
| | | clone_end = 3/gb = AI840191/gi = 5474404/ug = Mm.30015/len = 423/STRA = for | | | | | | |
| 9 | 97916_at | Cluster Incl AI116222: uc18c11.r1 Mus musculus cDNA, 5 end/clone = IMAGE-1398356/clone_end = 5/gb = AI116222/ | 422.2 | P | 394.6 | P | 322.8 | P |
| | | gi = 3516546/ug = Mm30116/len = 321/STRA = rev | | | | | | |
| 9 | 97922_at | Cluster Incl AW045597: UI-M-BH1-alg-a-01-0-UIs1 Mus musculus cDNA, 3 end/clone = UI-M-BH1-alg-a-01-0-UI/ | 562 | P | 683.1 | P | 568.3 | P |
| | | clone_end = 3/gb = AW045597/gi = 5906121/ug = Mm30166/len = 306/STRA = for | | | | | | |
| 9 | 98451_at | Cluster Incl AI843164: UI-M-AK1-aet-b-03-0-UIs1 Mus musculus cDNA, 3 end/clone = UI-M-AK1-aet-b-03-0-UI/ | 147.5 | P | 134.1 | P | 112 | P |
| | | clone_end = 3/gb = AI843164/gi = 5477377/ug = Mm34635/len = 375/STRA = rev | | | | | | |
| 9 | 98463_at | Cluster Incl AW124186: UI-M-BH2.1-aph-a-05-0-UIs1 Mus musculus cDNA, 3 end/clone = UI-M-BH2.1-aph-a-05-0-UI/ | 576.2 | P | 656.4 | P | 481.7 | P |
| | | clone_end = 3/gb = AW124186/gi = 6099716/ug = Mm34803/len = 237/STRA = for | | | | | | |
| 9 | 98996_at | Cluster Incl L29480 Mus musculus serine/threonine kinase (sak-b) mRNA, complete cds/cds = (205,1599)/gb = L29480/ | 220.5 | P | 263.9 | P | 189.6 | P |
| | | gi = 487871/ug = Mm3794/len = 1600/STRA = for | | | | | | |
| 9 | 99440_at | Cluster Incl Y07686 M musculus mRNA for NfiB3-protein, splice vanant (exon 9, 10 and 11 missing)/cds = (531,1793)/ | 1009.5 | P | 1095 | P | 921.6 | P |
| | | gb = Y07686/gi = 1524158/ug = Mm4025/len = 2514/STRA = rev | | | | | | |
| 9 | 99457_at | Cluster Incl X82786 Antigen identified by monoclonal antibody Ki 67/cds = (345,9161)/gb = X82786/gi = 1177527/ | 574.7 | P | 591.9 | P | 230.7 | P |
| | | ug = Mm4078/len = 9471/STRA = for | | | | | | |
| 9 | 99962_at | Cluster Incl D12644: Kinesin heavy chain member 2/cds = (504,2654)/gb = D12644/gi = 220467/ug = Mm4415/len = 3532/ | 66.8 | P | 84.9 | P | 51.7 | P |
| | | STRA = for | | | | | | |
| 9 | 100033_at | Cluster Incl X81143 MutS homolog 2 (E. coli)/cds = (39,2846)/gb = X81143/gi = 534944/ug = Mm4619/len = 3079/STRA = for | 504.6 | P | 351.9 | P | 485.7 | P |
| 9 | 100460_at | Cluster Incl L49502: TPR-containing, SH2-binding phosphoprotein/cds = (134,3655)/gb = L49502/gi = 1236238/ug = Mm489/ | 127 | P | 141.7 | P | 123.7 | P |
| | | len = 4201/STRA = rev | | | | | | |
| 9 | 100892_at | Cluster Incl AI850186 UI-M-BG1-aid-h-04-0-UIs1 Mus musculus cDNA, 3 end/clone = UI-M-BG1-aid-h-04-0-UI/ | 148.5 | P | 136.6 | P | 128.5 | P |
| | | clone_end = 3/gb = AI850186/gi = 5494092/ug = Mm5545/len = 271/STRA = for | | | | | | |
| 9 | 100979_at | Cluster Incl AW209763 ul46g10 y1 Mus musculus cDNA, 5 end/clone = IMAGE-2101410/clone_end = 5/gb = AW209763/ | 103.3 | P | 135.9 | P | 152.7 | P |
| | | gi = 6515703/ug = Mm6703/len = 440/STRA = for | | | | | | |
| 9 | 101367_at | Cluster Incl U60312: Dynactin 1/cds = (19,3864)/gb = U60312/gi = 2104494/ug = Mm6919/len = 4024/STRA = for | 388 | P | 473.4 | P | 438 | P |
| 9 | 101439_at | Cluster Incl AW122716 UI-M-BH22-aot-d-03-0-UIs1 Mus musculus cDNA, 3 end/clone = UI-M-BH22-aot-d-03-0-UI/ | 168.2 | P | 213.1 | P | 165.5 | P |
| | | clone_end = 3/gb = AW122716/gi = 6098246/ug = Mm7760/len = 409/STRA = for | | | | | | |
| 9 | 102883_at | Cluster Incl AW107702 um33b01 x1 Mus musculus cDNA, 3 end/clone = IMAGE-2236297/clone_end = 3/gb = AW107702/ | 143 | P | 85.7 | P | 114.9 | P |
| | | gi = 6078502/ug = Mm1506/len = 548/STRA = for | | | | | | |
| 9 | 104002_at | Cluster Incl AI153693 vz91d04 r1 Mus musculus cDNA, 5 end/clone = IMAGE-1344583/clone_end = 5/gb = AI153693/ | 1.6 | P | 47.3 | P | 43.5 | P |
| | | gi = 3682162/ug = Mm26595/len = 400/STRA = for | | | | | | |
| 9 | 104030_at | Cluster Incl AI848841 UI-M-AJ1-ahb-c-08-0-UIs1 Mus musculus cDNA, 3 end/clone = UI-M-AJ1-ahb-c-08-0-UI/ | 1012.8 | P | 928.7 | P | 486.2 | P |
| | | clone_end = 3/gb = AI848841/gi = 5492747/ug = Mm27042/len = 249/STRA = for | | | | | | |
| 9 | 104644_at | Cluster Incl D12646 Kinesin heavy chain member 4/cds = (731,4426)/gb = D12646/gi = 563772/ug = Mm3130/len = 4706/ | 206.9 | P | 128.1 | P | 118.9 | P |
| | | STRA = for | | | | | | |
| 9 | 92526_f_at | Cluster Incl AW122114 UI-M-BH22-aor-a-11-0-UIs1 Mus musculus cDNA, 3 end/clone = UI-M-BH22-aor-a-11-0-UI/ | 65.4 | P | 79.1 | P | 81.5 | P |
| | | clone_end = 3/gb = AW122114/gi = 6097531/ug = Mm4285/len = 271/STRA = for | | | | | | |
| 9 | 93236_s_at | Cluster Incl M13352 Thymidylate synthase/cds = (0,923)/gb = M13352/gi = 202049/ug = Mm5879/len = 924/STRA = for | 267.7 | P | 212.4 | P | 197.3 | P |
| 9 | 93859_at | Cluster Incl AI875598: uk50c11 x1 Mus musculus cDNA, 3 end/clone = IMAGE-1972436/clone_end = 3/gb = AI875598/ | 187.8 | P | 206.2 | P | 142.7 | P |
| | | gi = 5549647/ug = Mm87729/len = 513/STRA = rev | | | | | | |
| 9 | 95914_at | Cluster Incl AA177382: mt29b12.r1 Mus musculus cDNA, 5 end/clone = IMAGE-622463/clone_end = 5/gb = AA177382/ | 109 | P | 138.6 | P | 117.3 | P |
| | | gi = 1758617/ug = Mm24464/len = 542/STRA = for | | | | | | |
| 9 | 96594_at | Cluster Incl AA615831 vo90h05 r1 Mus musculus cDNA, 5 end/clone = IMAGE-1066425/clone_end = 5/gb = AA615831/ | 365.6 | P | 460.5 | P | 393.3 | P |
| | | gi = 2503059/ug = Mm30652/len = 257/STRA = for | | | | | | |
| 9 | 97164_at | Cluster Incl C77566 C77566 Mus musculus cDNA, 3 end/clone = J0033G09/clone_end = 3/gb = C77566/gi = 2517896/ | 314.7 | P | 356.8 | P | 322.6 | P |
| | | ug = Mm35516/len = 616/STRA = rev | | | | | | |
| 9 | 99366_at | Cluster Incl AI553536: vw39e06.x1 Mus musculus cDNA, 3 end/clone = IMAGE-1246210/clone_end = 3/gb = AI553536/ | 198.6 | P | 185.1 | P | 144.4 | P |
| | | gi = 4485899/ug = Mm5675/len = 408/STRA = rev | | | | | | |
| 9 | 102144_f_at | Cluster Incl AW048189 UI-M-BH1-amc-d-12-0-UIs1 Mus musculus cDNA, 3 end/clone = UI-M-BH1-amc-d-12-0-UI/ | 166.3 | P | 199 | P | 181.8 | P |
| | | clone_end = 3/gb = AW048189/gi = 5908718/ug = Mm44453/len = 141/STRA = rev | | | | | | |
| 9 | 93992_at | Cluster Incl AW123342: UI-M-BH2.1-apf-d-04-0-UIs1 Mus musculus cDNA, 3 end/clone = UI-M-BH2.1-apf-d-04-0-UI/ | 134.8 | P | 54.9 | A | 163.4 | P |
| | | clone_end = 3/gb = AW123342/gi = 6098872/ug = Mm21749/len = 405/STRA = for | | | | | | |
| 9 | 95447_at | Cluster Incl AA683761 vr04e08 r1 Mus musculus cDNA, 5 end/clone = IMAGE-1110854/clone_end = 5/gb = AA683761/ | 259.1 | P | 263.8 | A | 275 | P |
| | | gi = 2670347/ug = Mm24601/len = 335/STRA = for | | | | | | |
| 9 | 96002_at | Cluster Incl AW123936: UI-M-BH2.3-aqc-c-01-0-UIs1 Mus musculus cDNA, 3 end/clone = UI-M-BH2.3-aqc-c-01-0-UI/ | 252.7 | P | 233.4 | P | 200.6 | P |
| | | clone_end = 3/gb = AW123936/gi = 6099466/ug = Mm25331/len = 368/STRA = for | | | | | | |
| 9 | 96654_at | Cluster Incl AI852916: UI-M-BH0-aiz-b-03-0-UIs1 Mus musculus cDNA, 3 end/clone = UI-M-BH0-aiz-b-03-0-UI/ | 73.6 | P | 36.5 | P | 41.3 | A |
| | | clone_end = 3/gb = AI852916/gi = 5496822/ug = Mm27339/len = 392/STRA = for | | | | | | |
| 9 | 97261_at | Cluster Incl AF055664: Heat shock protein, DNAJ-like 2/cds = (91,1284)/gb = AF055664/gi = 3025863/ug = Mm27897/ | 393.9 | P | 297.5 | P | 344.3 | P |
| | | len = 2242/STRA = for | | | | | | |
| 9 | 94447_at | Cluster Incl AA726837 vu94h01 r1 Mus musculus cDNA, 5 end/clone = IMAGE-1209841/clone_end = 5/gb = AA726837/ | 73.3 | P | 54.7 | P | 58 | A |
| | | gi = 2744544/ug = Mm26780/len = 654/STRA = for | | | | | | |
| 9 | 95075_at | Cluster Incl AW229141 up29a12 y1 Mus musculus cDNA, 5 end/clone = IMAGE-2655742/clone_end = 5/gb = AW229141/ | 191.3 | P | 217.8 | P | 187.2 | P |
| | | gi = 6558373/ug = Mm29200/len = 287/STRA = for | | | | | | |
| 9 | 95712_at | Cluster Incl AW045261 UI-M-BH1-akn-a-07-0-UI s1 Mus musculus cDNA, 3 end/clone = UI-M-BH1-akn-a-07-0-UI/ | 185.4 | P | 131 | P | 107 | P |
| | | clone_end = 3/gb = AW045261/gi = 5905790/ug = Mm29709/len = 301/STRA = for | | | | | | |
| 9 | 99149_at | Cluster Incl AI851230 UI-M-BH0-ajx-h-10-0-UIs1 Mus musculus cDNA, 3 end/clone = UI-M-BH0-ajx-h-10-0-UI/ | 128.3 | P | 173.2 | P | 98.1 | A |
| | | clone_end = 3/gb = AI851230/gi = 5495136/ug = Mm41535/len = 416/STRA = for | | | | | | |
| 9 | 99616_s_at | Cluster Incl AI842156 UI-M-AN1-afj-e-11-0-UIs1 Mus musculus cDNA, 3 end/clone = UI-M-AN1-afj-e-11-0-UI/ | 237.5 | P | 199 | P | 196.5 | P |
| | | clone_end = 3/gb = AI842156/gi = 5476369/ug = Mm43017/len = 316/STRA = for | | | | | | |
| 9 | 100093_at | Cluster Incl X69025 PCTAIRE-motif protein kinase 1/cds = UNKNOWN/gb = X69025/gi = 53610/ug = Mm4597/len = 3078/ | 775.1 | A | 730.5 | P | 769.7 | P |
| | | STRA = for | | | | | | |
| 9 | 100128_at | Cluster Incl M38724: Cell division cycle control protein 2a/cds = (24,917)/gb = M38724/gi = 192533/ug = Mm.4761/len = 963/ | 110.1 | P | 91.4 | P | 139.4 | P |
| | | STRA = for | | | | | | |
| 9 | 101481_at | Cluster Incl AI852555: UI-M-BH0-aiw-g-04-0-UI.s1 Mus musculus cDNA, 3 end/clone = UI-M-BH0-aiw-g-04-0-UI/ | 89.7 | P | 115.4 | P | 100.1 | P |
| | | clone_end = 3/gb = AI852555/gi = 5496461/ug = Mm.7780/len = 351/STRA = for | | | | | | |
| 9 | 103582_r_at | Cluster Incl AI845633 UI-M-AQ1-adx-h-07-0-UI s1 Mus musculus cDNA, 3 end/clone = UI-M-AQ1-adx-h-07-0-UI/ | 139.8 | P | 122.2 | P | 118.4 | P |
| | | clone_end = 3/gb = AI845633/gi = 5489539/ug = Mm.19799/len = 430/STRA = for | | | | | | |
| 9 | 104373_at | Cluster Incl AW046876 UI-M-BH1-aid-c-02-0-UI s1 Mus musculus cDNA, 3 end/clone = UI-M-BH1-aki-c-02-0-UI/ | 51.2 | A | 60.1 | P | 44 | P |
| | | clone_end = 3/gb = AW046876/gi = 5907405/ug = Mm 22645/len = 525/STRA = for | | | | | | |
| 9 | 104547_at | Cluster Incl J00388 mouse dihydrofolate reductase gene/cds = (0,563)/gb = J00388/gi = 193340/ug = Mm 23695/len = 564/ | 52.6 | A | 72.2 | P | 35.5 | P |
| | | STRA = for | | | | | | |
| 9 | 94396_at | Cluster Incl AA647507: vq78d10 s1 Mus musculus cDNA, 5 end/clone = IMAGE-1108435/clone_end = 5/gb = AA647507/ | 295.3 | P | 346.8 | P | 313.2 | P |
| | | gi = 2573936/ug = Mm25709/len = 345/STRA = for | | | | | | |
| 9 | 95523_at | Cluster Incl AI839718 UI-M-AN0-acp-h-02-0-UI s1 Mus musculus cDNA, 3 end/clone = UI-M-AN0-acp-h-02-0-UI/ | 118.6 | P | 170.4 | P | 171.9 | A |
| | | clone_end = 3/gb = AI839718/gi = 5473931/ug = Mm.27579/len = 244/STRA = for | | | | | | |
| 9 | 97926_s_at | Cluster Incl U10374 Peroxisome proliferator activator receptor gamma/cds = (0,1427)/gb = U10374/gi = 507776/ | 77.9 | P | 90.7 | A | 150 | P |
| | | ug = Mm 3020/len = 1428/STRA = for | | | | | | |
| 9 | 99917_at | Cluster Incl U52951 Mus musculus putative transcriptional regulator mEnx-1 mRNA, complete cds/cds = (134,2374)/ | 286.6 | P | 222.9 | A | 223 | P |
| | | gb = U52951/gi = 1279908/ug = Mm4303/len = 2653/STRA = for | | | | | | |
| 9 | 102907_at | Cluster Incl AW125043: UI-M-BH2 1-apx-b-03-0-UI s1 Mus musculus cDNA, 3 end/clone = UI-M-BH2 1-apx-b-03-0-UI/ | 42.2 | P | 65.3 | M | 101.4 | P |
| | | clone_end = 3/gb = AW125043/gi = 6100573/ug = Mm158/len = 465/STRA = for | | | | | | |
| 9 | 102934_s_at | Cluster Incl L16926 Cell division cycle control protein 25C/cds = (481,1878)/gb = L16926/gi = 451133/ug = Mm16857/ | 158.8 | P | 64.2 | P | 71.4 | A |
| | | len = 2124/STRA = for | | | | | | |
| 9 | 103696_r_at | Cluster Incl AW047329 UI-M-BH1-amh-h-04-0-UI s1 Mus musculus cDNA, 3 end/clone = UI-M-BH1-amh-h-04-0-UI/ | 31.4 | P | 46 | P | 19.2 | A |
| | | clone_end = 3/gb = AW047329/gi = 5907858/ug = Mm.23994/len = 438/STRA = for | | | | | | |
| 9 | 103726_at | Cluster Incl AI842264 UI-M-AI1-afp-g-06-0-UI s1 Mus musculus cDNA, 3 end/clone = UI-M-AI1-afp-g-06-0-UI/clone_end = 3/ | 266 | P | 264.1 | P | 278 | P |
| | | gb = AI842264/gi = 5476477/ug = Mm24286/len = 384/STRA = for | | | | | | |
| 9 | 104194_at | Cluster Incl AF082567 Mus musculus hephaestin (heph) mRNA, complete cds/cds = (619,4092)/gb = AF082567/gi = 4322373/ | 63.5 | P | 17.1 | A | 30.4 | P |
| | | ug = Mm27494/len = 4804/STRA = for | | | | | | |
| 9 | 92191_at | Cluster Incl AI255450 ui85f10 x1 Mus musculus cDNA, 3 end/clone = IMAGE-1889227/clone_end = 3/gb = AI255450/ | 87.9 | P | 74.2 | A | 76.1 | P |
| | | gi = 3862975/ug = Mm 34009/len = 509/STRA = for | | | | | | |
| 9 | 95379_at | Cluster Incl AA245183 mw28g07 r1 Mus musculus cDNA, 5 end/clone = IMAGE-672060/clone_end = 5/gb = AA245183/ | 90.3 | P | 113 | P | 69.1 | P |
| | | gi = 1875900/ug = Mm 22988/len = 290/STRA = rev | | | | | | |
| 9 | 99365_at | Cluster Incl AA734487 vt94c10 r1 Mus musculus cDNA, 5 end/clone = IMAGE-1178802/clone_end = 5/gb = AA734487/ | 202.1 | P | 368 | P | 158.6 | A |
| | | gi = 2756154/ug = Mm 5662/len = 513/STRA = rev | | | | | | |
| 9 | 95498_at | Cluster Incl AI836029 UI-M-AQ0-aad-e-12-0-UI.s2 Mus musculus cDNA, 3 end/clone = UI-M-AQ0-aad-e-12-0-UI/ | 113.2 | P | 88.9 | A | 67.5 | A |
| | | clone_end = 3/gb = AI836029/gi = 5470207/ug = Mm 25143/len = 513/STRA = for | | | | | | |
| 9 | 96670_at | Cluster Incl AI841295 UI-M-AM0-adu-g-05-0-UI s1 Mus musculus cDNA, 3 end/clone = UI-M-AM0-adu-g-05-0-UI/ | 151 | P | 107.8 | P | 34.1 | A |
| | | clone_end = 3/gb = AI841295/gi = 5475508/ug = Mm 27395/len = 441/STRA = for | | | | | | |
| 9 | 99620_at | Cluster Incl AW060546 UI-M-BH1-ann-g-04-0-UI s1 Mus musculus cDNA, 3 end/clone = UI-M-BH1-ann-g-04-0-UI/ | 68.9 | A | 103.4 | P | 76.4 | P |
| | | clone_end = 3/gb = AW060546/gi = 6008297/ug = Mm 43213/len = 353/STRA = for | | | | | | |
| 9 | 100612_at | Cluster Incl K02927 Ribonucleotide reductase M1/cds = (81,2459)/gb = K02927/gi = 200764/ug = Mm 656/len = 2902/ | 387.3 | P | 202.2 | A | 354.2 | P |
| | | STRA = for | | | | | | |
| 9 | 103385_at | Cluster Incl U64033 Mus musculus Tera (Tera) mRNA, complete cds/cds = (81,914)/gb = U64033/gi = 1575504/ | 260.1 | P | 139.6 | P | 95.1 | P |
| | | ug = Mm.18637/len = 2720/STRA = for | | | | | | |
| 9 | 95032_at | Cluster Incl AA856349: vw99g06 r1 Mus musculus cDNA, 5 end/clone = IMAGE-1263130/clone_end = 5/gb = AA856349/ | 293.3 | P | 91.2 | A | 135.3 | P |
| | | gi = 2944651/ug = Mm 27529/len = 291/STRA = for | | | | | | |
| 9 | 95620_at | Cluster Incl AW120882 UI-M-BH2 3-aoa-h-10-0-UI.s1 Mus musculus cDNA, 3 end/clone = UI-M-BH2 3-aoa-h-10-0-UI/ | 180.9 | P | −32 | P | −15.2 | A |
| | | clone_end = 3/gb = AW120882/gi = 6096215/ug = Mm 2804/len = 327/STRA = for | | | | | | |
| 9 | 97345_at | Cluster Incl AI849333 UI-M-AJ1-aha-e-03-0-UI.s1 Mus musculus cDNA, 3 end/clone = UI-M-AJ1-ahn-e-03-0-UI/ | 270.5 | P | 277.9 | A | 116.7 | A |
| | | clone_end = 3/gb = AI849333/gi = 5493239/ug = Mm.29613/len = 548/STRA = for | | | | | | |
| 9 | 97421_at | Cluster Incl U42385: Fibroblast growth factor Inducible 16/cds = (45,1187)/gb = U42385/gi = 1353708/ug = Mm 2999/ | 114.3 | A | 202.1 | P | 99.8 | P |
| | | len = 2808/STRA = rev | | | | | | |
| 9 | 100413_at | Cluster Incl AB033168 Mus musculus mRNA for nuclear protein ZAP, complete cds/cds = (101,4261)/gb = AB033168/ | 273.9 | P | 211.9 | A | 207 | P |
| | | gi = 6016841/ug = Mm 46674/len = 4698/STRA = for | | | | | | |
| 9 | 101440_at | Cluster Incl AW047074 UI-M-BH1-alr-e-08-0-UI.s1 Mus musculus cDNA, 3 end/clone = UI-M-BH1-alr-e-08-0-UI/ | 164.9 | P | 150.6 | P | 106.3 | P |
| | | clone_end = 3/gb = AW047074/gi = 5907603/ug = Mm 778/len = 330/STRA = for | | | | | | |
| 9 | 101835_at | Cluster Incl U10484 Mus musculus lymphoid-restricted membrane protein(Jaw1) mRNA, complete cds/cds = (151,1770)/ | 12.8 | P | −156.7 | P | −151.2 | P |
| | | gb = U10484/gi = 505683/ug = Mm 843/len = 2023/STRA = for | | | | | | |
| 9 | 102646_at | Cluster Incl AW122284: UI-M-BH2 2-aos-b-02-0-UI s1 Mus musculus cDNA, 3 end/clone = UI-M-BH2-2-aos-b-02-0-UI/ | 126.4 | P | 63.6 | P | 63.7 | A |
| | | clone_end = 3/gb = AW122284/gi = 6097765/ug = Mm 13148/len = 336/STRA = for | | | | | | |
| 9 | 103939_at | Cluster Incl AW121399 UI-M-BH2 2-aom-h-02-0-UI s1 Mus musculus cDNA, 3 end/clone = UI-M-BH2.2-aom-h-02-0-UI/ | 74.6 | P | 32.5 | A | 23.3 | A |
| | | clone_end = 3/gb = AW121399/gi = 6096732/ug = Mm 25250/len = 550/STRA = for | | | | | | |
| 9 | 104247_at | Cluster Incl AI846291: UI-M-AK1-aez-f-04-0-UI.s1 Mus musculus cDNA, 3 end/clone = UI-M-AK1-aez-f-04-0-UI/ | 198.1 | P | 170 | P | 163.1 | P |
| | | clone_end = 3/gb = AI846291/gi = 5490197/ug = Mm.28335/len = 287/STRA = for | | | | | | |
| 9 | 104431_at | Cluster Incl D11091 Protein Kinase C, theta/cds = (88,2211)/gb = D11091/gi = 220573/ug = Mm.2921/len = 3313/STRA = for | 173.7 | P | 139.8 | A | 178.1 | P |
| 9 | 92468_at | Cluster Incl AF028722 Mus musculus fetal globin inducing factor mRNA, complete cds/cds = (135,851)/gb = AF028722/ | 105.5 | P | 82.1 | P | 154 | P |
| | | gi = 4103856/ug = Mm.42088/len = 1801/STRA = for | | | | | | |
| 9 | 98335_at | Cluster Incl X72711 Replication factor C, 140 KDa/cds = (79,3474)/gb = X72711/gi = 457741/ug = Mm 4280/len = 4679/ | 182.6 | P | 185.6 | A | 160.6 | P |
| | | STRA = for | | | | | | |
| 9 | 92788_f_at | Cluster Incl Y12474 M musculus mRNA for centrin gene/cds = (0,503)/gb = Y12474/gi = 2246423/ug = Mm.12481/len = 504/ | 460.8 | A | 552.4 | P | 398.7 | P |
| | | STRA = for | | | | | | |
| 9 | 98146_at | Cluster Incl AW120775: UI-M-BH2.3-any-f-10-0-UI.s1 Mus musculus cDNA, 3 end/clone = UI-M-BH2.3-any-f-10-0-UI/ | 211 | P | 178.1 | A | 239.6 | A |
| | | clone_end = 3/gb = AW120775/gi = 6096108/ug = Mm.35698/len = 463/STRA = for | | | | | | |
| 9 | 103354_at | Cluster Incl Z46966: M. musculus mRNA for imogen 44/cds = (45,1199)/gb = Z46966/gi = 1272668/ug = Mm.1841/len = 1267/ | 250.3 | P | 227.7 | A | 240 | A |
| | | STRA = for | | | | | | |
| 9 | 95586_at | Cluster Incl AF089751 Mus musculus ATP receptor P2X4 subunit mRNA, complete cds/cds = (31,1197)/gb = AF089751/ | 153.4 | P | 148.7 | P | 67.1 | M |
| | | gi = 4019303/ug = Mm 27861/len = 1944/STRA = for | | | | | | |
| 9 | 98409_at | Cluster Incl L00923 Mouse myosin I mRNA, complete cds/cds = (196,3435)/gb = L00923/gi = 200008/ug = Mm 3390/ | 179.6 | A | 164.1 | P | 112.6 | A |
| | | len = 4194/STRA = for | | | | | | |
| 9 | 98995_at | Cluster Incl X86925 M musculus mRNA for E2F-5 protein/cds = (15,1022)/gb = X86925/gi = 806571/ug = Mm.379/len = 1340/ | 16.9 | P | 29.4 | A | −7.4 | P |
| | | STRA = for | | | | | | |
| 9 | 99937_at | Cluster Incl Z16406 Mesenchyme homeobox 2/cds = (163,1074)/gb = Z16406/gi = 57949/ug = Mm 4346/len = 1440/ | 70.7 | P | 35.9 | M | 29.7 | A |
| | | STRA = for | | | | | | |
| 9 | 100014_at | Cluster Incl AI845038 UI-M-BG0-ahy-b-01-0-UI.s1 Mus musculus cDNA, 3 end/clone = UI-M-BG0-ahy-b-01-0-UI/ | 76.4 | P | 51.3 | P | 30.8 | A |
| | | clone_end = 3/gb = AI845038/gi = 5488944/ug = Mm 4557/len = 380/STRA = for | | | | | | |
| 9 | 101444_at | Cluster Incl U83176 Gene trap ROSA 26 antisense/cds = (90,1607)/gb = U83176/gi = 1778860/ug = Mm 781/len = 1974/ | 242.4 | P | 189.8 | A | 145.8 | A |
| | | STRA = for | | | | | | |
| 9 | 93436_i_at | Cluster Incl AI850509 UI-M-BG1-aih-h-10-0-UI s1 Mus musculus cDNA, 3 end/clone = UI-M-BG1-aih-h-10-0-UI/ | 57.5 | P | 18.7 | M | 0.5 | A |
| | | clone_end = 3/gb = AI850509/gi = 5494415/ug = Mm 65832/len = 453/STRA = for | | | | | | |
| 9 | 93470_at | Cluster Incl AF012129 Mus musculus putative DNA methyltransferase (Dnmt2) mRNA, complete cds/cds = (19,1266)/ | 74.6 | P | 53.6 | P | 39.6 | A |
| | | gb = AF012129/gi = 2636723/ug = Mm 6979/len = 1691/STRA = for | | | | | | |
| 9 | 100138_f_at | Cluster Incl X52102 M musculus p16K gene for 16 kDa protein/cds = (77,1798)/gb = X52102/gi = 53546/ug = Mm 4877/ | 179.8 | P | 96 | A | 89.9 | A |
| | | len = 2751/STRA = for | | | | | | |
| 9 | 100148_at | Cluster Incl U51037 Mus musculus 11-zinc-finger transcription factor (CTCF) mRNA, complete cds/cds = (306,2516)/ | 166.7 | P | 29.6 | A | 97.2 | A |
| | | gb = U51037/gi = 1256409/ug = Mm 493/len = 3840/STRA = for | | | | | | |
| 9 | 103081_at | Cluster Incl AA863928 vx15h09 r1 Mus musculus cDNA, 5 end/clone = IMAGE-1264577/clone_end = 5/gb = AA863928/ | 162.4 | M | 192.8 | M | 341.9 | P |
| | | gi = 2956407/ug = Mm 1717/len = 273/STRA = for | | | | | | |
| 9 | 95561_at | Cluster Incl AW120867 UI-M-BH2 3-aoa-g-06-0-UI s1 Mus musculus cDNA, 3 end/clone = UI-M-BH2 3-aoa-g-06-0-UI/ | 96 | P | 82.3 | P | 65.8 | A |
| | | clone_end = 3/gb = AW120867/gi = 6096200/ug = Mm 27777/len = 312/STRA = for | | | | | | |
| 9 | 98515_at | Cluster Incl AI844392 UI-M-AL1-ahp-d-12-0-UI s1 Mus musculus cDNA, 3 end/clone = UI-M-AL1-ahp-d-12-0-UI/ | 149.9 | A | 73.5 | P | 168.4 | P |
| | | clone_end = 3/gb = AI844392/gi = 5488298/ug = Mm 36088/len = 445/STRA = for | | | | | | |
| 9 | 98979_at | Cluster Incl U35249 Mus musculus CDK-activating kinase assembly factor p36/MAT1, complete cds/cds = (152,1081)/ | 51.8 | P | 29.1 | A | −1.6 | A |
| | | gb = U35249/gi = 1015998/ug = Mm 3688/len = 1409/STRA = for | | | | | | |
| 9 | 100955_at | Cluster Incl AA989957: ua55a07 r1 Mus musculus cDNA, 5 end/clone = IMAGE-1361364/clone_end = 5/gb = AA989957/ | 62.8 | P | 55.8 | A | 6.3 | A |
| | | gi = 3175321/ug = Mm 6476/len = 456/STRA = for | | | | | | |
| 9 | 102194_at | Cluster Incl AW122332 UI-M-BH2 2-aoa-f-10-0-UI s1 Mus musculus cDNA, 3 end/clone = UI-M-BH2 2-aoa-f-10-0-UI/ | 185.3 | M | 74.9 | M | 161.2 | P |
| | | clone_end = 3/gb = AW122332/gi = 6097817/ug = Mm 98631/len = 303/STRA = rev | | | | | | |
| 9 | 102293_at | Cluster Incl L03547 Early lymphoid specific transcription factor/cds = (38,1333)/gb = L03547/gi = 198286/ug = Mm 12396/ | 68.9 | P | 74.1 | A | 75.2 | P |
| | | len = 1550/STRA = rev | | | | | | |
| 9 | 104451_at | Cluster Incl AI852578 UI-M-BH0-aiu-a-08-0-UI s1 Mus musculus cDNA, 3 end/clone = UI-M-BH0-aiu-a-08-0-UI/ | 255.3 | M | 271.9 | P | 215.9 | A |
| | | clone_end = 3/gb = AI852578/gi = 5496484/ug = Mm.29433/len = 417/STRA = for | | | | | | |
| 9 | 93217_at | Cluster Incl AW049642 UI-M-BH1-and-g-02-0-UI s1 Mus musculus cDNA, 3 end/clone = UI-M-BH1-and-g-02-0-UI/ | 149 | M | 215.4 | M | 220.9 | P |
| | | clone_end = 3/gb = AW049642/gi = 5910171/ug = Mm 5710/len = 444/STRA = for | | | | | | |
| 9 | 93560_at | Cluster Incl AI845882 UI-M-AO1-aeg-h-05-0-UI s1 Mus musculus cDNA, 3 end/clone = UI-M-AO1-aeg-h-05-0-UI/ | 234.6 | P | 156.5 | A | 113.3 | A |
| | | clone_end = 3/gb = AI845882/gi = 5489788/ug = Mm.20309/len = 423/STRA = for | | | | | | |
| 9 | 94000_at | Cluster Incl X07698: Mouse mRNA for CD8 molecule Ly-3 chain/cds = (49,690)/gb = X07698/gi = 52954/ug = Mm 21764/ | 258.2 | A | 273.9 | P | 278.8 | A |
| | | len = 1111/STRA = for | | | | | | |
| 9 | 96656_at | Cluster Incl AI644179: vw55g04.x1 Mus musculus cDNA, 3 end/clone = IMAGE-1247766/clone_end = 3/gb = AI644179/ | 86.5 | A | 29.7 | M | 46.4 | A |
| | | gi = 4722654/ug = Mm 27339/len = 269/STRA = for | | | | | | |
| 9 | 99129_at | Cluster Incl AW124529 UI-M-BH2.1-apo-g-08-0-UI s1 Mus musculus cDNA, 3 end/clone = UI-M-BH2 1-apo-g-08-0-UI/ | 31.3 | A | 43.6 | A | 41.5 | A |
| | | clone_end = 3/gb = AW124529/gi = 6100059/ug = Mm 41043/len = 503/STRA = for | | | | | | |
| 9 | 101108_at | Cluster Incl AF034610 Mus musculus nuclear autoantigenic sperm protein mRNA, complete cds/cds = (91,2412)/ | −11.6 | A | 31.9 | A | 33.8 | A |
| | | gb = AF034610/gi = 2645972/ug = Mm 7516/len = 2543/STRA = for | | | | | | |
| 9 | 104296_at | Cluster Incl AI847802: UI-M-AK1-aev-g-02-0-UI s1 Mus musculus cDNA, 3 end/clone = UI-M-AK1-aev-g-02-0-UI/ | 107.7 | P | 68.7 | A | 79.6 | A |
| | | clone_end = 3/gb = AI847802/gi = 5491708/ug = Mm 22334/len = 520/STRA = for | | | | | | |
| 9 | 104535_at | Cluster Incl AF090430 Mus musculus ATP-dependent metalloprotease FlsH1 mRNA, complete cds/cds = (172,2319)/ | 68.5 | A | 62.8 | P | 43.6 | P |
| | | gb = AF090430/gi = 3600099/ug = Mm 23335/len = 2654/STRA = for | | | | | | |
| 9 | 96216_at | Cluster Incl AI848826 UI-M-AJ1-ahb-b-04-0-UI s1 Mus musculus cDNA, 3 end/clone = UI-M-AJ1-ahb-b-04-0-UI/ | 124.2 | A | 94.5 | A | 132.1 | P |
| | | clone_end = 3/gb = AI848826/gi = 5492732/ug = Mm 28677/len = 493/STRA = for | | | | | | |
| 9 | 102625_f_at | Cluster Incl AF080070: Mus musculus zinc finger protein 54 (Zfp54) mRNA, complete cds/cds = (121,2115)/gb = AF080070/ | 11.1 | A | 37.4 | A | 21 | A |
| | | gi = 3406675/ug = Mm 12916/len = 2267/STRA = rev | | | | | | |
| 9 | 93529_at | Cluster Incl AW125219 UI-M-BH2 1-aps-c-07-0-UI.s1 Mus musculus cDNA, 3 end/clone = UI-M-BH2 1-aps-c-07-0-UI/ | 143.1 | A | 113.8 | A | 147.8 | A |
| | | clone_end = 3/gb = AW125219/gi = 6100749/ug = Mm 19803/len = 345/STRA = for | | | | | | |
| 9 | 93758_at | Cluster Incl AA823653: vr69c07.s1 Mus musculus cDNA, 5 end/clone = IMAGE-1125900/clone_end = 5/gb = AA823653/ | 274.3 | A | 126.4 | A | 106.3 | A |
| | | gi = 2893521/ug = Mm.21140/len = 554/STRA = for | | | | | | |
| 9 | 94509_at | Cluster Incl AI851207 UI-M-BH0-ajx-f-06-0-UI s1 Mus musculus cDNA, 3 end/clone = UI-M-BH0-ajx-f-06-0-UI/clone_end = 3/ | 87.3 | A | 106.7 | A | 67.7 | A |
| | | gb = AI851207/gi = 5495113/ug = Mm 28968/len = 332/STRA = for | | | | | | |
| 9 | 96902_at | Cluster Incl AW121847 UI-M-BH2 3-aoi-a-12-0-UI.s1 Mus musculus cDNA, 3 end/clone = UI-M-BH2 3-aoi-a-12-0-UI/ | 200 | A | 205.8 | A | 200.1 | A |
| | | clone_end = 3/gb = AW121847/gi = 6097221/ug = Mm 3012/len = 241/STRA = for | | | | | | |
| 9 | 96924_at | Cluster Incl AI849719 UI-M-AL1-ahn-b-05-0-UI.s1 Mus musculus cDNA, 3 end/clone = UI-M-AL1-ahn-b-05-0-UI/ | 304.1 | A | 225.9 | A | 227.3 | A |
| | | clone_end = 3/gb = AI849719/gi = 5493625/ug = Mm 30160/len = 413/STRA = for | | | | | | |
| 9 | 99185_at | Cluster Incl AW047026 UI-M-BH1-alr-a-02-0-UI s1 Mus musculus cDNA, 3 end/clone = UI-M-BH1-alr-a-02-0-UI/ | 118.1 | A | 106.6 | P | 98.6 | A |
| | | clone_end = 3/gb = AW047026/gi = 5907555/ug = Mm 41874/len = 257/STRA = for | | | | | | |
| 9 | 100577_at | Cluster Incl M58558: Small nuclear ribonucleoprotein D1/cds = (46,405)/gb = M58558/gi = 200993/ug = Mm.603/len = 506/ | 506.9 | P | 494.4 | A | 436 | A |
| | | STRA = for | | | | | | |
| 9 | 104097_at | Cluster Incl AF002823 Budding inhibited by benzimidazoles 1 (S. cerevislae) homolog/cds = (60,3236)/gb = AF002823/ | −3.5 | M | −13.1 | A | −15.1 | A |
| | | gi = 2150135/ug = Mm 2185/len = 3370/STRA = for | | | | | | |
| 9 | 95544_at | Clustor Incl AI843150 UI-M-AK1-aes-h-12-0-UI s1 Mus musculus cDNA, 3 end/clone = UI-M-AK1-aes-h-12-0-UI/ | 33.9 | M | 98.3 | A | 112.2 | P |
| | | clone_end = 3/gb = AI843150/gi = 5477363/ug = Mm 27687/len = 229/STRA = for | | | | | | |
| 9 | 99541_at | Cluster Incl AJ223293 Mus musculus mRNA for kinesin-related mitotic motor protein/cds = (0,3044)/gb = AJ223293/ | 120.6 | P | 38.6 | A | 46.6 | A |
| | | gi = 4160555/ug = Mm.42203/len = 4412/STRA = for | | | | | | |
| 9 | 102303_f_at | Cluster Incl L28167: Zinc finger protein 61/cds = (927,2594)/gb = L28167/gi = 758660/ug = Mm.12832/len = 2606/STRA = for | 249.5 | A | 209.2 | A | 161.3 | A |
| 9 | 103685_at | Cluster Incl AI839232 UI-M-AKO-adh-d-11-0-UI.s1 Mus musculus cDNA, 3 end/clone = UI-M-AKO-adh-d-11-0-UI/ | 33.7 | A | 60.4 | A | 13.9 | A |
| | | clone_end = 3/gb = AI839232/gi = 5473445/ug = Mm 23897/len = 407/STRA = for | | | | | | |
| 9 | 93228_at | Cluster Incl U25691: Helicase, lymphoid specific/cds = (39,1850)/gb = U25691/gi = 805295/ug = Mm.57223/len = 1851/ | 9.2 | A | 54.8 | A | 8.6 | A |
| | | STRA = for | | | | | | |
| 9 | 100306_at | Cluster Incl AI510297 vx94c04 y1 Mus musculus cDNA, 5 end/clone = IMAGE-1293606/clone_end = 5/gb = AI510297/ | 74.6 | A | 62.9 | A | 121.4 | P |
| | | gi = 4409202/ug = Mm 86721/len = 434/STRA = for | | | | | | |
| 10 | 93055_at | Cluster Incl AW049376 UI-M-BH1-ane-c-05-0-UI s1 Mus musculus cDNA, 3 end/clone = UI-M-BH1-ane-c-05-0-UI/ | 155.4 | P | 139.4 | P | 116.6 | P |
| | | clone_end = 3/gb = AW049376/gi = 5909905/ug = Mm 15351/len = 492/STRA = for | | | | | | |
| 10 | 93258_at | Cluster Incl M28666 Hydroxymethylbilane synthase/cds = (0,1202)/gb = M28666/gi = 200231/ug = Mm 1710/len = 1203/ | 176.4 | P | 206.1 | P | 143.5 | P |
| | | STRA = for | | | | | | |
| 10 | 93496_at | Cluster Incl AI852098 UI-M-BH0-aja-h-11-0-UI s1 Mus musculus cDNA, 3 end/clone = UI-M-BH0-aja-h-11-0-UI/ | 711.4 | P | 796.6 | P | 604.1 | P |
| | | clone_end = 3/gb = AI852098/gi = 5496004/ug= Mm 19130/len= 504/STRA = for | | | | | | |
| 10 | 93521_at | Cluster Incl AW049194 UI-M-BH1-amq-g-12-0-UI.s1 Mus musculus cDNA, 3 end/clone = UI-M-BH1-amq-g-12-0-UI/ | 719.5 | P | 847.3 | P | 622.7 | P |
| | | clone_end = 3/gb = AW049194/gi = 5909723/ug = Mm 1963/len = 272/STRA = for | | | | | | |
| 10 | 93782_at | Cluster Incl AI844517: UI-M-AJ1-ahf-a-02-0-UI s2 Mus musculus cDNA, 3 end/clone = UI-M-AJ1-ahf-a-02-0-UI/ | 1087 | P | 1073.6 | P | 1019.2 | P |
| | | clone_end = 3/gb = AI844517/gi = 5488423/ug = Mm 21281/len = 504/STRA = for | | | | | | |
| 10 | 93831_at | Cluster Incl AI316087: uk62h02 y1 Mus musculus cDNA, 5 end/clone = IMAGE-1973619/clone_end = 5/gb = AI316087/ | 279.1 | P | 257.1 | P | 248.9 | P |
| | | gi = 4031354/ug = Mm 21559/len = 512/STRA = for | | | | | | |
| 10 | 94007_at | Cluster Incl AW122469 UI-M-BH2 2-aou-d-04-0-UI s1 Mus musculus cDNA, 3 end/clone = UI-M-BH2 2-aou-d-04-0-UI/ | 2743.9 | P | 2791.9 | P | 2439.6 | P |
| | | clone_end = 3/gb = AW122469/gi = 6097989/ug = Mm 21802/len = 224/STRA = for | | | | | | |
| 10 | 94225_at | Cluster Incl AI844679 UI-M-AL1-ahr-f-03-0-UI s1 Mus musculus cDNA, 3 end/clone = UI-M-AL1-ahr-f-03-0-UI/clone_end = 3/ | 418.9 | P | 414 | P | 277.5 | P |
| | | gb = AI844679/gi = 5488585/ug = Mm.22264/len = 475/STRA = for | | | | | | |
| 10 | 94257_at | Cluster Incl AI836117 UI-M-AQ0-aae-c-02-0-UI s2 Mus musculus cDNA, 3 end/clone = UI-M-AQ0-aae-c-02-0-UI/ | 167.5 | P | 367.6 | P | 221.5 | P |
| | | clone_end = 3/gb = AI836117/gi = 5470325/ug = Mm 2241/len = 289/STRA = for | | | | | | |
| 10 | 94286_at | Cluster Incl AW050340: UI-M-BH1-ang-c-08-0-UI s1 Mus musculus cDNA, 3 end/clone = UI-M-BH1-ang-c-08-0-UI/ | 439.1 | P | 382 | P | 345 | P |
| | | clone_end = 3/gb = AW050340/gi = 5910869/ug = Mm 22565/len = 492/STRA = for | | | | | | |
| 10 | 94853_at | Cluster Incl U29055 Guanine nuleotide binding protein, beta 1/cds = (39,1061)/gb = 32 U29055/gi = 868166/ug = Mm 2344/ | 740.9 | P | 716.8 | P | 539 | P |
| | | len = 1410/STRA = for | | | | | | |
| 10 | 94854_g_at | Cluster Incl U29055 Guanine nucleotide binding protein, beta 1/cds = (39,1061)/gb = U29055/gi = 868166/ug = Mm 2344/ | 675.6 | P | 798.5 | P | 649.1 | P |
| | | len = 1410/STRA = for | | | | | | |
| 10 | 94860_at | Cluster Incl AW121821 UI-M-BH2 3-anx-d-12-0-UI s2 Mus musculus cDNA, 3 end/clone = UI-M-BH2 3-anx-d-12-0-UI/ | 752.3 | P | 816.4 | P | 656.5 | P |
| | | clone_end = 3/gb = AW121821/gi = 6097189/ug = Mm 2368/len = 370/STRA = for | | | | | | |
| 10 | 94871_r_at | Cluster Incl AW124226: UI-M-BH2 1-aph-d-10-0-UI.s1 Mus musculus cDNA, 3 end/clone = UI-M-BH2 1-aph-d-10-0-UI/ | 636 | P | 682.2 | P | 543.4 | P |
| | | clone_end = 3/gb = AW124226/gi = 6099756/ug = Mm 23755/len = 382/STRA = for | | | | | | |
| 10 | 94895_at | Cluster Incl AW122948: UI-M-BH2. 1-aoz-c-05-0-UI s2 Mus musculus cDNA, 3 end/clone = UI-M-BH2.1-aoz-c-05-0-UI/ | 373.5 | P | 448 | P | 387.8 | P |
| | | clone_end = 3/gb = AW122948/gi = 6098478/ug = Mm 23998/len = 451/STRA = for | | | | | | |
| 10 | 94896_at | Cluster Incl D90151 Mouse mRNA for CArG-binding factor-A, complete cds/cds = (193,1050)/gb = D90151/gi = 220362/ | 956.1 | P | 839.2 | P | 760.3 | P |
| | | ug = Mm.24/len = 1595/STRA = for | | | | | | |
| 10 | 94910_at | Cluster Incl AW120739: UI-M-BH2.3-any-c-04-0-UI.s1 Mus musculus cDNA, 3 end/clone = UI-M-BH2.3-any-c-04-0-UI/ | 175.1 | P | 141.7 | P | 107.9 | P |
| | | clone_end = 3/gb = AW120739/gi = 6096072/ug = Mm 24105/len = 445/STRA = for | | | | | | |
| 10 | 95419_at | Cluster Incl M29260 Mouse histone 1-0 gene, 5 end promoter region/cds = (266,268)/gp = M29260/gi = 193849/ | 2821.6 | P | 2314.8 | P | 1231.6 | P |
| | | ug = Mm 24350/len = 269/STRA = for | | | | | | |
| 10 | 95460_at | Clustor Incl U70736 Mus musculus 38 kDa Mov34 homolog mRNA, complete cds/cds = (123,1127)/gb = U70736/ | 452.3 | P | 554.6 | P | 503.7 | P |
| | | gi = 2360946/ug = Mm 2472/len = 1190/STRA = for | | | | | | |
| 10 | 96065_at | Cluster Incl D88769 Mus musculus mRNA fo latexin, complete cds/cds = (99,767)/gp = U88769/gi = 1669620/ug = Mm 2632 | 244.1 | P | 181.6 | P | 129.3 | P |
| | | len = 996/STRA = for | | | | | | |
| 10 | 96606_at | Cluster Incl AB025217: Mus musculus mRNA for Sid470p, complete cds/cds = (55, 642)/gb = AB025217/gi = 5931554/ | 242.4 | P | 328.8 | P | 181.6 | P |
| | | ug = Mm 27065/len = 782/STRA = for | | | | | | |
| 10 | 96626_at | Cluster Incl AI843401 UI-M-AQ1-adz-h-01-0-UI.s1 Mus musculus cDNA, 3 end/clone = UI-M-AQ1-adz-h-01-0-UI/ | 537.1 | P | 601.7 | P | 513.2 | P |
| | | clone_end = 3/gb = AI843401/gi = 5477614/ug = Mm 27182/len = 336/STRA = for | | | | | | |
| 10 | 96700_r_at | Cluster Incl AW060594 UI-M-BH1-anq-d-05-0-UI.s1 Mus musculus cDNA, 3 end/clone = UI-M-BH1-anq-d-05-0-UI/ | 67.5 | P | 102.1 | P | 63.1 | P |
| | | clone_end = 3/gb = AW060594/gi = 6008345/ug = Mm 27560/len = 245/STRA = for | | | | | | |
| 10 | 97272_at | Cluster Incl M99167 Mouse rearranged RNA binding protein (FLI-2) mRNA, complete cds/cds = (59,1021)/gb = M99167/ | 680.1 | P | 701 | P | 417.3 | P |
| | | gi = 193323/ug = Mm 27927/len = 1443/STRA = for | | | | | | |
| 10 | 97312_at | Cluster Incl AB014464 Mus musculus mRNA for MGC-24v, complete cds/cds = (0,593)/gb = AB014464/gi = 3892655/ | 1437.2 | P | 2149.8 | P | 1306.2 | P |
| | | ug = Mm 28083/len = 594/STRA = for | | | | | | |
| 10 | 97818_at | Cluster Incl AI838691 UI-M-AO0-aca-g-07-0-UI.s1 Mus musculus cDNA, 3 end/clone = UI-M-AO0-aca-g-07-0-UI/ | 733.9 | P | 710.1 | P | 638.1 | P |
| | | clone_end = 3/gb = AI838691/gi = 5472904/ug = Mm 28196/len = 387/STRA = for | | | | | | |
| 10 | 97843_at | Cluster Incl AI834866: UI-M-AN1-afe-a-11-0-UI s1 Mus musculus cDNA, 3 end/clone = UI-M-AN1-afe-a-11-0-UI/ | 709 | P | 517.1 | P | 469 | P |
| | | clone_end = 3/gb = AI834866/gi = 5469079/ug = Mm 28261/len = 488/STRA = for | | | | | | |
| 10 | 98917_at | Clustor Incl AA823202: vw41h10r1 Mus musculus cDNA, 5 end/clone = IMAGE-1246435/clone_end = 5/gb = AA823202/ | 508.1 | P | 558.7 | P | 394.7 | P |
| | | gi = 2893070/ug = Mm 2862/len = 363/STRA = for | | | | | | |
| 10 | 98929_at | Cluster Incl AI846452: UI-M-AQ1-aee-f-09-0-UI.s1 Mus musculus cDNA, 3 end/clone = UI-M-AQ1-aee-f-09-0-UI/ | 392.3 | P | 353 | P | 287.8 | P |
| | | clone_end = 3/gb = AI846452/gi = 5490358/ug = Mm.28664/len = 283/STRA = for | | | | | | |
| 10 | 94562_at | Cluster Incl AI843968: UI-M-AP1-agh-e-10-0-UI s1 Mus musculus cDNA, 3 end/clone = UI-M-AP1-agh-e-10-0-UI/ | 586.9 | P | 510.7 | P | 403 | P |
| | | clone_end = 3/gb = AI843968/gi = 5478181/ug = Mm 29114/len = 334/STRA = for | | | | | | |
| 10 | 95153_at | Cluster Incl AI845988: UI-M-AP1-agl-d-01-0-UI s1 Mus musculus cDNA, 3 end/clone = UI-M-AP1-agl-d-01-0-UI/ | 206.2 | P | 313.6 | P | 170.7 | P |
| | | clone_end = 3/gb = AI845988/gi = 5489894/ug = Mm 29476/len = 435/STRA = for | | | | | | |
| 10 | 95650_at | Cluster Incl U95114 Mus musculus fertilization antigen-1 mRNA, complete cds/cds = (82,576)/gb = U95114/gi = 2098784/ | 1082.6 | P | 923.4 | P | 893.6 | P |
| | | ug = Mm.29504/len = 642/STRA = for | | | | | | |
| 10 | 95665_at | Cluster Incl AI852087 UI-M-BH0-aja-g-04-0-UI s1 Mus musculus cDNA, 3 end/clone = UI-M-BH0-aja-g-04-0-UI/ | 328.1 | P | 261.3 | P | 370.7 | P |
| | | clone_end = 3/gb = AI852087/gi = 5495993/ug = Mm 29571/len = 260/STRA = for | | | | | | |
| 10 | 95754_at | Cluster Incl AI838216 UI-M-AL0-abx-c-12-0-UI s1 Mus musculus cDNA, 3 end/clone = UI-M-AL0-abx-c-12-0-UI/ | 715.6 | P | 736.2 | P | 295 | P |
| | | clone_end = 3/gb = AI838216/gi = 5472429/ug = Mm 29791/len = 474/STRA = for | | | | | | |
| 10 | 96243_f_at | Cluster Incl AW120804 UI-M-BH2 3-aoa-a-08-0-UI s1 Mus musculus cDNA, 3 end/clone = UI-M-BH2 3-aoa-a-08-0-UI/ | 248 | P | 217.6 | P | 206.7 | P |
| | | clone_end = 3/gb = AW120804/gi = 6096137/ug = Mm 29803/len = 289/STRA = for | | | | | | |
| 10 | 96257_at | Cluster Incl AW124376 UI-M-BH2 1-ape-a-11-0-UI s1 Mus musculus cDNA, 3 end/clone = UI-M-BH2 1-ape-a-11-0-UI/ | 564.1 | P | 674 | P | 532 | P |
| | | clone_end = 3/gb = AW124376/gi = 6099906/ug = Mm 29822/len = 213/STRA = for | | | | | | |
| 10 | 97488_at | Cluster Incl AI595893: uk19g08 x1 Mus musculus cDNA, 3 end/clone = IMAGE-1969502/clone_end = 3/gb = AI595893/ | 663.3 | P | 652 | P | 538.6 | P |
| | | gi = 4604941/ug = Mm 3094/len = 448/STRA = for | | | | | | |
| 10 | 98586_at | Cluster Incl AW107230: um12f03 x1 Mus musculus cDNA, 3 end/clone = IMAGE-2182877/clone_end = 3/gb = AW107280/ | 236.7 | P | 339.3 | P | 185.2 | P |
| | | gi = 6078030/ug = Mm.3797/len = 574/STRA = for | | | | | | |
| 10 | 99162_at | Cluster Incl AW122398: UI-M-BH2 2-aow-d-11-0-UI s1 Mus musculus cDNA, 3 end/clone = UI-M-BH2 2-aow-d-11-0-UI/ | 201 | P | 277.3 | P | 170.1 | P |
| | | clone_end = 3/gb = AW122398/gi = 6097899/ug = Mm 41670/len = 506/STRA = for | | | | | | |
| 10 | 99575_at | Cluster Incl AA517835 vh82b01 r1 Mus musculus cDNA, 3 end/clone = IMAGE-893449/clone_end = 3/gb = AA517835/ | 1333.3 | P | 1590.3 | P | 1167.9 | P |
| | | gi = 2257359/ug = Mm 4230/len = 440/STRA = for | | | | | | |
| 10 | 100073_at | Cluster Incl AW045965 UI-M-BH1-aku-h-09-0-UI s1 Mus musculus cDNA, 3 end/clone = UI-M-BH1-aku-h-09-0-UI/ | 202.6 | P | 160.8 | P | 145.9 | P |
| | | clone_end = 3/gb = AW045965/gi = 5906494/ug = Mm 45225/len = 554/STRA = for | | | | | | |
| 10 | 101096_s_at | Cluster Incl AF023482 Mus musculus HS1-associating protein (mHAX-1s) mRNA, complete cds/cds = (108,950)/ | 331.7 | P | 351.5 | P | 191.3 | P |
| | | gb = AF023482/gi = 2522493/ug = Mm 7401/len = 1121/STRA = for | | | | | | |
| 10 | 101101_at | Cluster Incl Z67746 M musculus mRNA for phosphatase 2A catalytic subnit, isotype beta/cds = (0,929)/gb = Z67746/ | 591.3 | P | 635.8 | P | 578.5 | P |
| | | gi = 1246796/ug = Mm 7418/len = 930/STRA = for | | | | | | |
| 10 | 101960_at | Cluster Incl AI842208 UI-M-AI1-afm-f-10-0-UI s1 Mus musculus cDNA, 3 end/clone = UI-M-AI1-afm-f-10-0-UI/clone_end = 3/ | 468.8 | P | 358.2 | P | 384.9 | P |
| | | gb = AI842208/gi = 5476421/ug = Mm 9257/len = 382/STRA = for | | | | | | |
| 10 | 102019_at | Cluster Incl AA666635 vm50b08 r1 Mus musculus cDNA, 5 end/clone = IMAGE-1001655/clone_end = 5/gb = AA666835/ | 530.7 | P | 388.5 | P | 429.7 | P |
| | | gi = 2625336/ug = Mm 10076/len = 203/STRA = for | | | | | | |
| 10 | 102039_at | Cluster Incl AI850881 UI-M-BH0-ajt-f-06-UI s1 Mus musculus cDNA, 3 end/clone = UI-M-BH0-ajt-1-06-0-UI/clone_end = 3/ | 292.7 | P | 365.1 | P | 208.8 | P |
| | | gb = AI850881/gi = 5494787/ug = Mm 10182/len = 471/STRA = for | | | | | | |
| 10 | 103047_at | Cluster Incl AF031128 Mus musculus peroxisome membrane protein PEX2 mRNA, complete cds/cds = (501,1418)/ | 329.3 | P | 300.2 | P | 277 | P |
| | | gb = AF031128/gi = 2623263/ug = Mm 16453/len = 1749/STRA = for | | | | | | |
| 10 | 103428_at | Cluster Incl AW049684 UI-M-BH1-ani-b-12-0-UI s1 Mus musculus cDNA, 3 end/clone = UI-M-BH1-ani-b-12-0-UI/ | 314.4 | P | 231 | P | 238.9 | P |
| | | clone_end = 3/gb = AW049684/gi = 5910213/ug = Mm.19000/len = 346/STRA = for | | | | | | |
| 10 | 103848_at | Cluster Incl AW121188 UI-M-BH2.3-aok-b-04-0-UI.s1 Mus musculus cDNA, 3 end/clone = UI-M-BH2.3-aok-b-04-0-UI/ | 352.6 | P | 453.1 | P | 244.7 | P |
| | | clone_end = 3/gb = AW121188/gi = 6096521/ug = Mm.21048/len = 477/STRA = for | | | | | | |
| 10 | 103861_s_at | Cluster Incl AA388099: vc88g07.r1 Mus musculus cDNA, 5 end/clone = IMAGE-790140/clone_end = 5/gb = AA388099/ | 252 | P | 346.7 | P | 202.6 | P |
| | | gi = 2041044/ug = Mm.21103/len = 564/STRA = for | | | | | | |
| 10 | 104297_at | Cluster Incl AW124742 UI-M-BH2 1-aqa-d-07-0-UI s1 Mus musculus cDNA, 3 end/clone = UI-M-BH2 1-aqa-d-07-0-UI/ | 346.6 | P | 327.7 | P | 275.9 | P |
| | | clone_end = 3/gb = AW124742/gi = 6100272/ug = Mm.22336/len = 466/STRA = for | | | | | | |
| 10 | 94360_at | Cluster Incl AA672650 vp57b04 r1 Mus musculus cDNA, 3 end/clone = IMAGE-1080751/clone_end = 3/gb = AA672650/ | 240.4 | P | 308.7 | P | 187.8 | P |
| | | gi = 2644867/ug = Mm.25263/len = 408/STRA = for | | | | | | |
| 10 | 94362_at | Cluster Incl AI843682 UI-M-AK1-aer-c-04-0-UI.s1 Mus musculus cDNA, 3 end/clone = UI-M-AK1-aer-c-04-0-UI/ | 1386.9 | P | 1458.4 | P | 1044.1 | P |
| | | clone_end = 3/gb = AI843682/gi = 5477895/ug = Mm 25265/len = 160/STRA = for | | | | | | |
| 10 | 94925_at | Cluster Incl AA754865: vu57f07 r1 Mus musculus cDNA, 5 end/clone = IMAGE-1194949/clone_end = 5/gb = AA754865/ | 210.1 | P | 219.2 | P | 197.8 | P |
| | | gi = 2802063/ug = Mm.26644/len = 324/STRA = for | | | | | | |
| 10 | 95525_at | Cluster Incl AI850793 UI-M-BG1-all-e-06-0-UI s1 Mus musculus cDNA, 3 end/clone = UI-M-BG1-all-e-06-0-UI/ | 349.9 | P | 327.9 | P | 303.9 | P |
| | | clone_end = 3/gb = AI850793/gi = 5494699/ug = Mm.27592/len = 517/STRA = for | | | | | | |
| 10 | 95539_at | Cluster Incl AA798971 vv95e03.r1 Mus musculus cDNA, 5 end/clone = IMAGE-1230172/clone_end = 5/gb = AA798971/ | 190.4 | P | 211.2 | P | 170 | P |
| | | gi = 2861926/ug = Mm.27668/len = 449/STRA = for | | | | | | |
| 10 | 96146_at | Cluster Incl D83745: Mus musculus mRNA for ANA, complete cds/cds = (173,931)/gb = D83745/gi = 3256228/ug = Mm.2823/ | 548.5 | P | 481.8 | P | 444.9 | P |
| | | len = 1380/STRA = for | | | | | | |
| 10 | 96744_at | Cluster Incl AW061051 UI-M-BH1-akk-f-05-0-UI s2 Mus musculus cDNA, 3 end/clone = UI-M-BH1-akk-f-05-0-UI/ | 502.9 | P | 293.1 | P | 284.3 | P |
| | | clone_end = 3/gb = AW061051/gi = 6008802/ug = Mm 28950/len = 517/STRA = for | | | | | | |
| 10 | 96754_s_at | Cluster Incl AI844751 UI-M-AL1-ahq-e-01-0-UI.s1 Mus musculus cDNA, 3 end/clone = UI-M-AL1-ahq-e-01-0-UI/ | 1035 | P | 1171.5 | P | 857.8 | P |
| | | clone_end = 3/gb = AI844751/gi = 5488657/ug = Mm 28982/len = 437/STRA = for | | | | | | |
| 10 | 96804_at | Cluster Incl AI854811 UI-M-BH0-aka-g-11-0-UI s1 Mus musculus cDNA, 3 end/clone = UI-M-BH0-aka-g-11-0-UI/ | 307.4 | P | 334.8 | P | 251.4 | P |
| | | clone_end = 3/gb = AI854811/gi = 5498717/ug = Mm 29203/len = 366/STRA = rev | | | | | | |
| 10 | 97380_at | Cluster Incl AI197431 uf13e07.r1 Mus musculus cDNA, 3 end/clone = IMAGE-1851204/clone_end = 5/gb = AI197431/ | 218.8 | P | 367.2 | P | 365.2 | P |
| | | gi = 3750037/ug = Mm 29748/len = 331/STRA = for | | | | | | |
| 10 | 97390_at | Cluster Incl AW125294 UI-M-BH2.1-apy-b-10-0-UI s1 Mus musculus cDNA, 3 end/clone = UI-M-BH2.1-apy-b-10-0-UI/ | 314.6 | P | 247.6 | P | 218.3 | P |
| | | clone_end = 3/gb = AW125294/gi = 6100824/ug = Mm 29800/len = 345/STRA = for | | | | | | |
| 10 | 97979_at | Cluster Incl AI849126: UI-M-AJ1-agy-a-10-0-UI s1 Mus musculus cDNA, 3 end/clone = UI-M-AJ1-agy-a-10-0-UI/ | 248 | P | 264.1 | P | 208.9 | P |
| | | clone_end = 3/gb = AI849126/gi = 5493032/ug = Mm 3121/len = 489/STRA = for | | | | | | |
| 10 | 99499_at | Cluster Incl AB023619 Mus musculus mRNA, for methyltransferase related protein, complete cds/cds = (47,853)/ | 311.3 | P | 315.3 | P | 281.3 | P |
| | | gb = AB023619/gi = 4512324/ug = Mm 41667/len = 887/STRA = for | | | | | | |
| 10 | 99529_f_at | Cluster Incl AB025011: Mus musculus mRNA for Trif-d, complete cds/cds = (303,590)/gb = AB025011/gi = 4586406/ | 110.1 | P | 143.5 | P | 129.5 | P |
| | | ug = Mm.42154/len = 1078/STRA = for | | | | | | |
| 10 | 100425_at | Cluster Incl U25685: Mus musculus protein tyrosine kinase (syk) mRNA complete cds/cds = (0,1889)/gb = U25685/ | 561 | P | 560.1 | P | 467.3 | P |
| | | gi = 818860/ug = Mm 4708/len = 1890/STRA = for | | | | | | |
| 10 | 101944_at | Cluster Incl U89352: Mus musculus lysophospholipase I mRNA, complete cds/cds = (0,692)/gb = U89352/gi = 1864158/ | 791.9 | P | 859.2 | P | 389.7 | P |
| | | ug = Mm 95696/len = 693/STRA = for | | | | | | |
| 10 | 101946_at | Cluster Incl AA840463 vx93d09.r1 Mus musculus cDNA, 5 end/clone = IMAGE-1282769/clone_end = 5/gb = AA840463/ | 253.1 | P | 290.9 | P | 183.4 | P |
| | | gi = 2916122/ug = Mm 95696/len = 430/STRA = for | | | | | | |
| 10 | 102271_at | Cluster Incl AW125462 UI-M-BH2 2-aqm-a-11-0-UI s1 Mus musculus cDNA, 3 end/close = UI-M-BH2 2-aqm-a-11-0-UI/ | 604.5 | P | 691.4 | P | 321.9 | P |
| | | clone_end = 3/gb = AW125462/gi = 6100992/ug = Mm.1144/len = 439/STRA = for | | | | | | |
| 10 | 103242_at | Cluster Incl AW123834 UI-M-BH2.1-apm-h-06-0-UI s1 Mus musculus cDNA, 3 end/clone = UI-M-BH2 1-apm-h-06-0-UI/ | 145.2 | P | 479.7 | P | 127.2 | P |
| | | clone_end = 3/gb = AW123834/gi = 6099364/ug = Mm.20417/len = 403/STRA = for | | | | | | |
| 10 | 103754_at | Cluster Incl AI847826 UI-M-AM1-afu-a-09-0-UI s1 Mus musculus cDNA, 3 end/clone = UI-M-AM1-afu-a-09-0-UI/ | 386.8 | P | 402.8 | P | 269.3 | P |
| | | clone_end = 3/gb = AI847826/gi = 5491732/ug = Mm 24529/len = 301/STRA = for | | | | | | |
| 10 | 104447_r_at | Cluster Incl AI595812: uj69f12.x1 Mus musculus cDNA, 3 end/clone = IMAGE-1925231/clone_end = 3/gb = AI595812/ | 72.7 | P | 42.5 | P | 12.8 | P |
| | | gi = 4604860/ug = Mm 29409/len = 596/STRA = for | | | | | | |
| 10 | 92389_at | Cluster Incl AB025411: Mus musculus mRNA for Ten-m2, complete cds/cds = (112,8406)/gb = AB025411/gi = 4760777/ | 676.9 | P | 806 | P | 493.2 | P |
| | | ug = Mm 40599/len = 8797/STRA = rev | | | | | | |
| 10 | 93215_at | Cluster Incl AF061346 Tumor necrosis factor induced protein 1/cds = (0,950)/gb = AF061346/gi = 3114712/ug = Mm.57088/ | 214.7 | P | 213.9 | P | 218.3 | P |
| | | len = 951/STRA = for | | | | | | |
| 10 | 93430_at | Cluster Incl AF000236 Chemokine orphan receptor 1/cds = (88,1176)/gb = AF000236/gi = 2439995/ug = Mm 6522/len = 1861/ | 117.8 | P | 74.6 | P | 41.8 | P |
| | | STRA = for | | | | | | |
| 10 | 93918_at | Cluster Incl AA673500 vp49g08 r1 Mus musculus cDNA, 3 end/clone = IMAGE-1080062/clone_end = 3/gb = AA673500/ | 204.8 | P | 255.3 | P | 184.8 | P |
| | | gi = 2646782/ug = Mm 89836/len = 357/STRA = for | | | | | | |
| 10 | 94189_at | Cluster Incl AB011665 Mus musculus mRNA for BAZF, complete cds/cds = (24,1448)/gb = AB01165/gi = 3287500/ | 917.2 | P | 466.5 | P | 330.2 | P |
| | | ug = Mm 12930/len = 3345/STRA = for | | | | | | |
| 10 | 95885_at | Cluster Incl AA177621 mt32e09 r1 Mus musculus cDNA, 5 end/clone = IMAGE-622792/clone_end = 5/gb = AA177621/ | 233.2 | P | 157.8 | P | 100.8 | P |
| | | gi = 1758855/ug = Mm 23986/len = 517/STRA = for | | | | | | |
| 10 | 97104_g_at | Cluster Incl AF031380 Mus musculus KOI-4 gene, partial cds/cds = (0,102)/gb = AF031380/gi = 2623675/ug = Mm 32715/ | 344.8 | P | 310 | P | 223.2 | P |
| | | len = 103/STRA = rev | | | | | | |
| 10 | 97770_s_at | Cluster Incl AA733372 vt74c06 r1 Mus musculus cDNA, 5 end/clone = IMAGE-1176874/clone_end = 5/gb = AA733372/ | 123.8 | P | 152.5 | P | 75.5 | P |
| | | gi = 2755039/ug = Mm 4176/len = 316/STRA = for | | | | | | |
| 10 | 98356_at | Cluster Incl AW125421 UI-M-BH2 3-aqh-f-02-0-UI s1 Mus musculus cDNA, 3 end/clone = UI-M-BH2 3-aqh-f-02-0-UI/ | 184 | P | 206.9 | P | 151 | P |
| | | clone_end = 3/gb = AW125421/gi = 6100951/ug = Mm.43948/len = 570/STRA = rev | | | | | | |
| 10 | 93567_at | Cluster Incl AW122536 UI-M-BH2 2-aox-c-06-0-UI s1 Mus musculus cDNA, 3 end/clone = UI-M-BH2 2-aox-c-06-0-UI/ | 190.2 | P | 139.7 | A | 139.3 | P |
| | | clone_end = 3/gb = AW122536/gi = 6098060/ug = Mm.20399/len = 523/STRA = for | | | | | | |
| 10 | 97847_at | Cluster Incl AJ237847: Mus musculus mRNA for heterogeneous nuclear ribonucleoprotein G, splice variant 1/ | 339.2 | P | 305.8 | P | 272.4 | P |
| | | cds = (153,1328)/gb = AJ237847/gi = 5579010/ug = Mm 28275/len = 2074/STRA = for | | | | | | |
| 10 | 102319_at | Cluster Incl AF062484 Mus musculus SDP8 mRNA, complete cds/cds = (64,561)/gb = AF062484/gi = 3126980/ | 42.2 | P | 49.3 | P | 8.7 | A |
| | | ug = Mm.10704/len = 617/STRA = for | | | | | | |
| 10 | 95573_at | Cluster Incl AW122821: UI-M-BH2 1-aoz-c-02-0-UI s1 Mus musculus cDNA, 3 end/clone = UI-M-BH2 1-aoz-c-02-0-UI/ | 541.2 | P | 410.3 | P | 194.2 | P |
| | | clone_end = 3/gb = AW122821/gi = 6098351/ug = Mm 27809/len = 329/STRA = rev | | | | | | |
| 10 | 98006_at | Cluster Incl D13546 Mus musculus mRNA for DNA polymerase alpha associated subunit, complete cds/cds = (235,2037)/ | 165.6 | P | 223 | P | 171.6 | P |
| | | gb = D13546/gi = 303658/ug = Mm 320/len = 2352/STRA = for | | | | | | |
| 10 | 99032_at | Cluster Incl AF009246 Mus musculus ras-related protein (DEXRAS1) mRNA, complete cds/cds = (141,983)/gb = AF009246/ | 150.6 | P | 151.2 | P | 142.9 | P |
| | | gi = 2253712/ug = Mm 3903/len = 1623/STRA = for | | | | | | |
| 10 | 99078_at | Cluster Incl AI839522: UI-M-ANO-acn-d-09-UI s1 Mus musculus cDNA, 3 end/clone = UI-M-ANO-acn-d-09-0-UI/ | 555.2 | P | 303.8 | P | 283.7 | P |
| | | clone_end = 3/gb = AI839522/gi = 5473735/ug = Mm 4022/len = 481/STRA = for | | | | | | |
| 10 | 103681_at | Cluster Incl AW049274: UI-M-BH1-anb-d-07-0-UI s1 Mus musculus cDNA, 3 end/clone = UI-M-BH1-anb-d-07-0-UI/ | 256.3 | P | 295.5 | P | 422.9 | P |
| | | clone_end = 3/gb = AW049274/gi = 5909768/ug = Mm 23885/len = 471/STRA = for | | | | | | |
| 10 | 104396_at | Cluster Incl AI019327 ub 19g08 r1 Mus musculus cDNA, 5 end/clone = IMAGE-1378238/clone_end = 5/gb = AI019327/ | 81 | P | 247.9 | P | 84.7 | A |
| | | gi = 3233683/ug = Mm 28617/len = 265/STRA = for | | | | | | |
| 10 | 93680_at | Cluster Incl D89728 Mus musculus mRNA for LOK, complete cds/cds = (99,2999)/gb = D89728/gi = 2723474/ug = Mm 8235/ | 222.2 | P | 206.3 | P | 89.9 | P |
| | | len = 5002/STRA = for | | | | | | |
| 10 | 94709_at | Cluster Incl D88611: Glial cells missing homolog (Drosophila), related sequence 2/cds = (241,1755)/gb = D88611/ | 267 | P | 264.8 | P | 162.9 | P |
| | | gi = 1769815/ug = Mm 1399/len = 3118/STRA = for | | | | | | |
| 10 | 99338_at | Cluster Incl AA674798: vm74h03 s1 Mus musculus cDNA, 5 end/clone = IMAGE-1004021/clone_end = 5/gb = AA674798/ | 197.3 | P | 245.5 | P | 156.4 | P |
| | | gi = 2652035/ug = Mm.5300/len = 466/STRA = rev | | | | | | |
| 10 | 93248_at | Cluster Incl AI050598 ub33h10 r1 Mus musculus cDNA, 5 end/clone = IMAGE-1379587/clone_end = 5/gb = AI050598/ | 227.1 | P | 198.8 | P | 159.8 | M |
| | | gi = 3299715/ug = Mm.16925/len = 487/STRA = for | | | | | | |
| 10 | 96678_at | Cluster Incl AW260761 um87b01 y1 Mus musculus cDNA, 5 end/clone = IMAGE-2331721/clone_end = 5/gb = AW260761/ | 64.3 | A | 98.3 | P | −131 | P |
| | | gi = 6633742/ug = Mm.27427/len = 336/STRA = for | | | | | | |
| 10 | 96148_at | Cluster Incl AA028728 mi16d09.r1 Mus musculus cDNA, 5 end/clone = IMAGE-463697/clone_end = 5/gb = AA028728/ | 130.5 | M | 205.5 | P | 99.6 | P |
| | | gi = 1494856/ug = Mm.3572/len = 1061/STRA = for | | | | | | |
| 10 | 100570_at | Cluster Incl U27462 Mus musculus BS4 peptide mRNA, complete cds/cds = (247,2280)/gb = U27462/gi = 863013/ | 354.2 | P | 337.2 | P | 147.5 | P |
| | | ug = Mm 5856/len = 3312/STRA = for | | | | | | |
| 10 | 103863_at | Cluster Incl AW049769 UI-M-BH1-anm-c-05-0-UI.s1 Mus musculus cDNA, 3 end/clone = UI-M-BH1-anm-c-05-0-UI/ | 811 | P | 850.9 | A | 443.8 | P |
| | | clone_end = 3/gb = AW049769/gi = 5910298/ug = Mm 21104/len = 543/STRA = rev | | | | | | |
| 10 | 98992_at | Cluster Incl AB030483: Mus musculus mRNA for B9, complete cds/cds = (37,651)/gb = AB030483/gi = 5668700/ | 212.9 | P | 178.3 | P | 130.1 | A |
| | | ug = Mm 37756/len = 817/STRA = for | | | | | | |
| 10 | 99028_at | Cluster Incl AI788534 uk46b08.x1 Mus musculus cDNA, 3 end/clone = IMAGE-1972023/clone_end = 3/gb = AI788534/ | 121.8 | P | 130.5 | P | 113.5 | P |
| | | gi = 5336250/ug = Mm.38831/len = 682/STRA = for | | | | | | |
| 10 | 99041_at | Cluster Incl Y09973: M. musculus mRNA for TAF168/cds = (0,1760)/gb = Y09973/gi = 1842205/ug = Mm.39082/len = 1761/ | 333.1 | A | 404.8 | P | 98 | P |
| | | STRA = for | | | | | | |
| 10 | 100929_at | Cluster Incl AW046351: UI-M-BH1-ali-a-02-0-UI.s1 Mus musculus cDNA, 3 end/clone = UI-M-BH1-ali-a-02-0-UI/ | 93.4 | P | 99.4 | P | 66.5 | P |
| | | clone_end = 3/gb AW046351/gi = 5906880/ug = Mm 6128/len = 503/STRA = for | | | | | | |
| 10 | 102724_at | Cluster Incl AI608324: vf77b06 y1 Mus musculus cDNA, 5 end/clone = IMAGE-849779/clone_end = 5/gb = AI608324/ | 128.8 | P | 159.1 | P | 85.5 | A |
| | | gi = 4617491/ug = Mm 1436/len = 474/STRA = for | | | | | | |
| 10 | 103476_at | Cluster Incl AA674794: vm74g09.s1 Mus musculus cDNA, 5 end/clone = IMAGE-1004032/clone_end = 5/gb = AA674794/ | 355.8 | P | 329.2 | P | 117.5 | A |
| | | gi = 2652031/ug = Mm 21926/len = 175/STRA = for | | | | | | |
| 10 | 98306_g_at | Cluster Incl Y11245 M musculus mRNA for winged-helix transcription factor/cds = (79,2352)/gb = Y11245/gi = 2065248/ | 609.5 | P | 421.3 | P | 382.7 | P |
| | | ug = Mm 42148/len = 2673/STRA = for | | | | | | |
| 10 | 100395_at | Cluster Incl X99104 M musculus mRNA for gli2 gene/cds = UNKNOWN/gb = X99104/gi = 1707589/ug = Mm.89929/len = 5849/ | 191.8 | P | 211.5 | P | 186.2 | P |
| | | STRA = for | | | | | | |
| 10 | 92827_at | Cluster Incl AF064553 Mus musculus NSD1 protein mRNA, complete cds/cds = (1099,8865)/gb = AF064553/gi = 3329464/ | 64 | A | 36.1 | A | 35.9 | P |
| | | ug = Mm 12964/len = 10019/STRA = for | | | | | | |
| 10 | 97539_at | Cluster Incl AI854416 UI-M-BH0-alt-b-02-0-UI s1 Mus musculus cDNA, 3 end/clone = UI-M-BH0-alt-b-02-0-UI/clone_end = 3/ | 25.3 | A | 47.2 | A | 72.5 | P |
| | | gb = AI854416/gi = 5498322/ug = Mm 33202/len = 504/STRA = for | | | | | | |
| 10 | 100122_at | Cluster Incl L34290 Mus musculus guanine nucleotide regulatory protein mRNA, complete cds/cds = (30,1091)/gb = L34290/ | 191.7 | P | 228.1 | P | 152.8 | A |
| | | gi = 1237255/ug = Mm 4702/len = 1803/STRA = for | | | | | | |
| 10 | 101003_at | Cluster Incl X53824 Splicing factor, arginine/serine-rich 3 (SRp20)/cds = (62,556)/gb = X53824/gi = 55439/ug = Mm 6787/ | 186.1 | P | 199.3 | P | 129.1 | P |
| | | len = 1239/STRA = for | | | | | | |
| 10 | 102809_s_at | Cluster Incl M12056 Mouse rearranged Ick gene encoding lymphocte-specific protein tyrosine kinase/cds = (163,1692)/ | 83.4 | M | 73.4 | P | 67.6 | P |
| | | gb = M12056/gi = 198763/ug = Mm 142/len = 2100/STRA = for | | | | | | |
| 10 | 103872_r_at | Cluster Incl AA667323: vv29b11.r1 Mus musculus cDNA, 5 end/clone = IMAGE-1223805/clone_end = 5/gb = AA667323/ | 19.2 | A | 84.5 | P | −11.1 | P |
| | | gi = 2626024/ug = Mm 21167/len = 210/STRA = for | | | | | | |
| 10 | 104153_at | Cluster Incl AW047743 UI-M-BH1-alk-b-07-0-UI s1 Mus musculus cDNA, 3 end/clone = UI-M-BH1-alk-b-07-0-UI/ | 243.6 | P | 251.5 | A | 184.1 | A |
| | | clone_end = 3/gb = AW047743/gi = 5908272/ug = Mm 22199/len = 339/STRA = for | | | | | | |
| 10 | 103516_at | Cluster Incl AF031572 Mus musculus seven-pass transmembrane receptor precursor (Celsr1) mRNA, complete cds/ | 307.1 | A | 303.9 | A | 260.2 | P |
| | | cds = (7,9111)/gb = AF031572/gi = 3800735/ug = Mm 22680/len = 10919/STRA = for | | | | | | |
| 10 | 104176_at | Cluster Incl AI850941 UI-M-BH0-akd-d-10-0-UI s1 Mus musculus cDNA, 3 end/clone = UI-M-BH0-akd-d10-0-UI/ | 129.1 | A | 146.1 | A | 167.4 | P |
| | | clone_end = 3/gb = AI850941/gi = 5494847/ug = Mm 27261/len = 377/STRA = for | | | | | | |
| 10 | 92914_at | Cluster Incl M18400: Mouse homeo box (Hox2 3) region/cds = (0,253)/gb = M18400/gi = 193939/ug = Mm 4822/len = 254/ | 361.5 | A | 479.9 | M | 195.7 | P |
| | | STRA = for | | | | | | |
| 10 | 93212_at | Cluster Incl Z97207 Mus musculus partial mRNA, for B-IND1 protein (B-ind1 gene)/cds = (0,570)/gb = Z97207/gi = 5777593/ | 54.1 | P | 105 | P | 64.2 | P |
| | | ug = Mm.57023/len = 571/STRA = for | | | | | | |
| 10 | 93481_at | Cluster Incl AI846720 UI-M-AN1-afi-h-09-0-UI s1 Mus musculus cDNA, 3 end/clone = UI-M-AN1-afi-b-09-0-UI/ | 138 | A | 506.8 | P | 298.7 | M |
| | | clone_end = 3/gb = AI846720/gi = 5490626/ug Mm 7124/len = 161/STRA = for | | | | | | |
| 10 | 98818_at | Cluster Incl X04435: Glucocorticold receptor 1/cds = (18,2369)/gb = X04435/gi = 51057/ug = Mm 4918/len = 2575/STRA = for | 49.3 | P | 70.8 | P | 54.7 | P |
| 10 | 93056_g_at | Cluster Incl AW049376 UI-M-BH1-ane-c-05-0-UI s1 Mus musculus cDNA, 3 end/clone = UI-M-BH1-ane-c-05-0-UI/ | 19.2 | P | 74.1 | A | −25.5 | A |
| | | clone_end = 3/gb = AW049376/gi = 5909905/ug = Mm 15351/len = 492/STRA = for | | | | | | |
| 10 | 97822_at | Cluster Incl AW122689 UI-M-BH2 2-aot-a-10-0-UI s1 Mus musculus cDNA, 3 end/clone = UI-M-BH2 2-aot-a-10-0-UI/ | 538.8 | A | 656.9 | P | 533.4 | P |
| | | clone_end = 3/gb = AW122689/gi = 6098319/ug = Mm 28202/len = 467/STRA = rev | | | | | | |
| 10 | 98886_at | Cluster Incl AI835412 UI-M-AQ0-aaj-d-12-0-UI s1 Mus musculus cDNA, 3 end/clone = UI-M-AQ0-aaj-d-12-0-UI/ | 575 | P | 342.1 | A | 331.4 | P |
| | | clone_end = 3/gb = AI835412/gi = 5469625/ug = Mm 28536/len = 355/STRA = for | | | | | | |
| 10 | 95122_g_at | Cluster Incl AW060791 UI-M-BH1-anu-f-12-0-UI s1 Mus musculus cDNA, 3 end/clone = UI-M-BH1-anu-f-12-0-UI/ | 91.3 | P | 53 | A | 27.8 | A |
| | | clone_end = 3/gb = AW060791/gi = 6008542/ug = Mm 29390/len = 519/STRA = for | | | | | | |
| 10 | 99163_at | Cluster Incl AI844232 UI-M-AL1-ahk-f-04-0-UI.s1 Mus musculus cDNA, 3 end/clone = UI-M-AL1-ahk-f-04-0-UI/ | 309.9 | P | 302.9 | P | 30.2 | M |
| | | clone_end = 3/gb = AI844232/gi = 5488138/ug = Mm 41671/len = 242/STRA = for | | | | | | |
| 10 | 94401_s_at | Cluster Incl AA986548 ue14g03 x1 Mus musculus cDNA, 3 end/clone = IMAGE-1480372/clone_end = 3/gb AA986548/ | 40.1 | P | 24.8 | P | 21.4 | A |
| | | gi = 3168202/ug = Mm 25793/len = 543/STRA = for | | | | | | |
| 10 | 98003_at | Cluster Incl AF038149 Mus musculus paired immonoglobutin-like receptor B (Pirb) gene, complete cds/cds = (0,2525)/ | 346.1 | A | 400.6 | P | 160.4 | A |
| | | gb = AF038149/gi = 3135465/ug = Mm.3189/len = 2526/STRA = for | | | | | | |
| 10 | 101945_g_at | Cluster Incl U89352: Mus musculus lysophospholipase I mRNA, complete cds/cds = (0,692)/gb = U89352/gi = 1864158/ | 75.1 | A | 69.3 | P | 14.4 | A |
| | | ug = Mm.95696/len = 693/STRA = for | | | | | | |
| 10 | 102277_at | Cluster Incl M36514.Mouse zinc finger protein (mkr3) mRNA, end/cds = (0,1286)/gb = M36514/gi = 191507/ug = Mm 1176/ | 37.9 | M | 103.3 | A | 22.5 | A |
| | | len = 2470/STRA = for | | | | | | |
| 10 | 103272_at | Cluster Incl AF091998: Mus musculus calpain Lp82 mRNA, complete cds/cds = (37,2166)/gb = AF091998/gi = 3661584/ | 502.5 | P | 463.6 | A | 245 | P |
| | | ug = Mm 20863/len = 2334/STRA = for | | | | | | |
| 10 | 93410_at | Cluster Incl AW120840 UI-M-BH2 3-aoa-e-01-0-UI s1 Mus musculus cDNA, 3 end/clone = UI-M-BH2 3-aoa-e-01-0-UI/ | 335.9 | P | 225.4 | P | 182.6 | M |
| | | clone_end = 3/gb = AW120840/gi = 6096173/ug = Mm.6378/len = 371/STRA = for | | | | | | |
| 10 | 100371_at | Cluster Incl U65316: Mus musculus hnRNP A1 (Hnrpa1) gene, alternative splicing region, exon 6 to partial exon 8, and | 133.6 | A | 172.1 | P | 93.6 | A |
| | | partial cds/cds = (0,381)/gb = U65316/gi = 1507693/ug Mm.88834/len = 382/STRA = for | | | | | | |
| 10 | 92559_at | Cluster Incl U12884: Mus musculus C57BL/6 vascuar cell adhesion molecule-1 truncated form T-VCAM-1 (VCAM-1) | 25 | A | 22.7 | A | 14 | M |
| | | mRNA, complete cds/cds = (43,1080)/gb = U12884/gi = 531884/ug Mm.1021/len = 1332/STRA = for | | | | | | |
| 10 | 92857_at | Cluster Incl AI853960 UI-M-BH0-aiv-d-02-0-UI s1 Mus musculus cDNA, 3 end/clone = UI-M-BH0-aiv-d-02-0-UI/ | 226.2 | P | 124.6 | A | −81 | A |
| | | clone_end = 3/gb = AI853960/gi = 5497866/ug = Mm.13917/len = 388/STRA = for | | | | | | |
| 10 | 93091_s_at | Cluster Incl M63503: Mouse keratinocyte growth factor receptor mRNA, complete cds/cds = (675,2798)/gb = M63503/ | 96.5 | P | 91.5 | P | −60.3 | P |
| | | gi = 198593/ug Mm 16340/len 3037/STRA = for | | | | | | |
| 10 | 97860_at | Cluster Incl AJ002198.Nucleosome assembly protein 1 like 4/cds = (66,1193)/gb = AJ002198/gi = 3319976/ug Mm 2831/ | 523.8 | A | 467.7 | M | 490.8 | M |
| | | len = 2301/STRA = for | | | | | | |
| 10 | 98016_at | Cluster Incl AA981266 vx80f03 r1 Mus musculus cDNA, 5 end/clone = IMAGE-1279613/clone_end = 5/gb = AA981268/ | 166.4 | A | 187.9 | A | 79.7 | A |
| | | gi = 3159804/ug = Mm 3235/len = 424/STRA = for | | | | | | |
| 10 | 102696_s_at | Cluster Incl AI747899: ul03g02.y1 Mus musculus cDNA, 5 end/clone = IMAGE-2065010/clone_end = 5/gb = AI747899/ | 60.2 | M | 98.1 | P | 78 | A |
| | | gi = 5126163/ug = Mm.14147/len = 564/STRA = rev | | | | | | |
| 10 | 102699_at | Cluster Incl J03368: Myxovirus (influenza virus) resistance 2/cds = UNKNOWN/gb = J03368/gi = 199921/ug = Mm.14157/ | 315.4 | M | 316.1 | P | 217.8 | A |
| | | len = 2426/STRA = for | | | | | | |
| 10 | 103928_at | Cluster Incl AI788018 uf16c04.y1 Mus musculus cDNA, 5 end/clone = IMAGE-2087718/clone_end = 5/gb = AI788018/ | 111.7 | P | 159.8 | P | 97.1 | A |
| | | gi = 5335734/ug = Mm 25096/len = 488/STRA = for | | | | | | |
| 10 | 104170_at | Cluster Incl AF003115 Mus musculus JIP-1 (JIP-1) mRNA, complete cds/cds = (137,2119)/gb = AF03115/gi = 2316073/ | 886.6 | P | 602.2 | A | 268.4 | P |
| | | ug = Mm.2720/len = 2790/STRA = for | | | | | | |
| 10 | 93400_at | Cluster Incl U88990 Apoptosis Inhibitor 3/cds = (671,2161)/gb = U88990/gi = 2138318/ug = Mm 6299/len = 2691/STRA = for | 117 | P | 120.1 | P | 111.7 | A |
| 10 | 93911_at | Cluster Incl AI315090: uj23g10 x1 Mus musculus cDNA, 3 end/clone = IMAGE-1920834/clone_end = 3/gb = AI315090/ | 264.7 | A | 169 | A | 2.6 | A |
| | | gi = 4030357/ug = Mm.89567/len = 467/STRA = for | | | | | | |
| 10 | 101231_at | Cluster Incl C78334 C78334 Mus musculus cDNA, 3 end/clone = J0047A07/clone_end = 3/gb = C78334/gi = 2518664/ | 160.3 | P | 159 | M | 112.3 | A |
| | | ug = Mm.5703/len = 571/STRA = rev | | | | | | |
| 10 | 102093_f_at | Cluster Incl AI461979: ub69a03 x1 Mus musculus cDNA, 3 end/clone = IMAGE-1382965/clone_end = 3/gb = AI461979/ | 32.9 | A | 78.4 | A | 49.9 | A |
| | | gi = 4316009/ug = Mm 42326/len = 272/STRA = rev | | | | | | |
| 10 | 102163_at | Cluster Incl X60388 M musculus SmN pseudogene/cds = (0,725)/gb = X60388/gi = 54118/ug = Mm 46814/len = 726/ | 454.2 | P | 437.7 | A | 341 | A |
| | | STRA = for | | | | | | |
| 10 | 92841_l_at | Cluster Incl X51429 Chromogranin B/cds = (52,2085)/gb = X51429/gi = 50408/ug = Mm 1339/len = 2340/STRA = for | 110.9 | A | 124.5 | P | 110 | A |
| 10 | 96046_at | Cluster Incl X96207 M musculus mRNA for histone deacetylase/cds = (17,1465) gb = X98207/gi = 1771285/ug = Mm 2602/ | 224.2 | M | 222.3 | M | 164.9 | M |
| | | len = 1977/STRA = for | | | | | | |
| 10 | 102758_at | Cluster Incl AJ006486 Mus musculus mRNA for RNA binding protein/cds = (117,3470)/gb = AJ006486/gi = 4883436/ | 75.3 | A | 115 | P | 51 | A |
| | | ug = Mm 12931/len = 3593/STRA = for | | | | | | |
| 10 | 103393_at | Cluster Incl AA738776 W67d07 r1 Mus musculus cDNA, 5 end/clone = IMAGE-1227469/clone_end = 5/gb = AA738776/ | 92.9 | M | 88.3 | A | 26.9 | A |
| | | gi = 2776028/ug = Mm 18659/len = 385/STRA = for | | | | | | |
| 10 | 102631_at | Cluster Incl AB008674 Mus musculus mRNA for mBlm, complete cds/cds = (342,4592)/gb = AB008674/gi = 3370995/ | 68.2 | A | 78 | A | 30.1 | A |
| | | ug = Mm 12932/len = 4763/STRA = for | | | | | | |
| 10 | 103200_at | Cluster Incl AA711773 vu58g05 r1 Mus musculus cDNA, 5 end/clone = IMAGE-1195640/clone_end = 5/gb = AA711773/ | 79.3 | P | 73.3 | A | 46.9 | A |
| | | gi = 2721691/ug = Mm 1902/len = 473/STRA = for | | | | | | |
| 10 | 95244_at | Cluster Incl D10632 Zinc finger protein 40/cds = (222,2408)/gb = D10632/gi = 286104/ug = Mm 21025/len = 2754/STRA = for | 35.4 | P | 54.5 | P | 42.3 | A |
| 10 | 101677_at | Cluster Incl AI450336 mu27g11 x1 Mus musculus cDNA, 3 end/clone = IMAGE-640676/clone_end = 3/gb = AI450336/ | 99.7 | A | 247.7 | P | 95.8 | A |
| | | gi = 4296290/ug = Mm 32307/len = 451/STRA = rev | | | | | | |
| 11 | 92553_at | Cluster Incl AB025408 Mus musculus mRNA for sid478p, complete cds/cds = (74,922)/gb = AB025408/gi = 5931564/ | 1152.6 | P | 451.5 | P | 1103 | P |
| | | ug = Mm.1017/len = 1056/STRA = for | | | | | | |
| 11 | 92589_at | Cluster Incl AI846545 UI-M-AN1-aff-g-12-0-UI s1 Mus musculus cDNA, 3 end/clone = UI-M-AN1-aff-g-12-0-UI/ | 1018.3 | P | 856 | P | 879 | P |
| | | clone_end = 3/gb = AI846545/gi = 5490451/ug = Mm 10623/len = 354/STRA = for | | | | | | |
| 11 | 92595_r_at | Cluster Incl M61215 Mus musculus ferrochelatase mRNA, complete cds/cds = (92,1354)/gb = M61215/gi = 19362/ | 156.6 | P | 146.4 | P | 122.1 | P |
| | | ug = Mm.1070/len = 2897/STRA = for | | | | | | |
| 11 | 92608_at | Cluster Incl D88793 Cysteine rich protein/cds = (130,711)/gb = D88793/gi = 1695703/ug = Mm 10919/len = 938/STRA = for | 1482.2 | P | 819.1 | P | 782.6 | P |
| 11 | 92632_at | Cluster Incl AI842328: UI-M-AM1-afz-b-11-0-UI s1 Mus musculus cDNA, 3 end/clone = UI-M-AM1-afz-b-11-0-UI/ | 1332.4 | P | 730.3 | P | 848 | P |
| | | clone_end = 3/gb = AI842328/gi = 5476506/ug = Mm 1147/len = 389/STRA = for | | | | | | |
| 11 | 92636_f_at | Cluster Incl U11027 Mus musculus C57BL/6J sec61 protein complex gamma subunit mRNA, complete cds/cds = (3,209)/ | 2223.9 | P | 1211.2 | P | 1608.8 | P |
| | | gb = U11027/gi = 506791/ug = Mm.1164/len = 367/STRA = for | | | | | | |
| 11 | 92646_at | Cluster Incl U84903 Ribosomal protein L23/cds = (61,501)/gb = U84903/gi = 2565133/ug = Mm 12144/len = 582/STRA = for | 1291.1 | P | 755 | P | 963 | P |
| 11 | 92798_at | Cluster Incl AA870675 vq24f11.r1 Mus musculus cDNA, 5 end/clone = IMAGE-1095213/clone_end = 5/gb = AA870675/ | 3024.7 | P | 2116.7 | P | 2581.6 | P |
| | | gi = 2966120/ug = Mm.12677/len = 405/STRA = for | | | | | | |
| 11 | 92799_g_at | Cluster Incl AA870675 vq24f11.r1 Mus musculus cDNA, 5 end/clone = IMAGE-1095213/clone_end = 5/gb = AA870675/ | 3151.5 | P | 2165.9 | P | 2655.3 | P |
| | | gi = 2966120/ug = Mm 12677/len = 405/STRA = for | | | | | | |
| 11 | 92810_at | Cluster Incl AI842259 UI-M-AI1-afp-f-09-0-UI s1 Mus musculus cDNA, 3 end/clone = UI-M-AI1 -afp-f-09-0-UI/clone_end = 3/ | 504.1 | P | 316 | P | 346.4 | P |
| | | gb = AI842259/gi = 5476472/ug = Mm.12775/len = 389/STRA = for | | | | | | |
| 11 | 92845_at | Cluster Incl AI843232 UI-M-AK1-aet-h-03-0-UI s1 Mus musculus cDNA, 3 end/clone = UI-M-AK1-aet-h-03-0-UI/ | 590.8 | P | 264.1 | P | 225.8 | P |
| | | clone_end = 3/gb = AI843232/gi 5477445/ug = Mm 13445/len = 396/STRA = for | | | | | | |
| 11 | 93057_at | Cluster Incl AW122989 UI-M-BH2.1-apb-b-08-0-UI s1 Mus musculus cDNA, 3 end/clone = UI-M-BH2 1-apb-b-08-0-UI/ | 3101.5 | P | 2871 | P | 3034.9 | P |
| | | clone_end = 3/gb = AW122989/gi = 6098519/ug = Mm 1538/len = 479/STRA = for | | | | | | |
| 11 | 93094_at | Cluster Incl U88588 Cerebellar degeneration-related 2/cds = (153,1520)/gb = U88588/gi = 1857920/ug = Mm 1640/len = 2432/ | 234 | P | 138.8 | P | 108.2 | P |
| | | STRA = for | | | | | | |
| 11 | 93102_f_at | Cluster Incl U20365: Actin, gamma 2, smooth muscle, enteric/cds = (36,1217)/gb = U20365/gi = 950001/ug = Mm 16562/ | 1485.7 | P | 914.3 | P | 1638 | P |
| | | len = 1232/STRA = for | | | | | | |
| 11 | 93119_at | Cluster Incl X53157 Mouse mRNA for mitochondrial cytochrome c oxidase subunit Vb/cds = (12,398)/gb = X53157/gi = 50530/ | 2889.3 | P | 1982.8 | P | 2616.4 | P |
| | | ug = Mm 16769/len = 475/STRA = for | | | | | | |
| 11 | 93126_at | Cluster Incl X04591 Mouse mRNA 3 region for brain creatine kinase B/cds = (0,86) gb = X04591/gi = 50413/ug = Mm 16831/ | 1082.1 | P | 532.1 | P | 510 | P |
| | | len = 285/STRA = for | | | | | | |
| 11 | 93276_at | Cluster Incl U90123.Hematological and neurological and neurological expressed sequence 1/cds = (95,559) | 1414.7 | P | 831.1 | P | 780 | P |
| | | gb = U90123/gi = 1864164/ | | | | | | |
| | | ug = Mm 1775/len = 1384/STRA = for | | | | | | |
| 11 | 93356_at | Cluster Incl D26091 Mus musculus mRNA for mCDC47, complete cds/cds = (106,2265)/gb = D26091/gi = 1136746/ | 550.3 | P | 490.3 | P | 390.2 | P |
| | | ug = Mm.18923/len = 2396/STRA = for | | | | | | |
| 11 | 93495_at | Cluster Incl U96746 Mus musculus antioxidant enzyme AOE372 mRNA, complete cds/cds = (90,914) gb = U96746/ | 844.6 | P | 232.2 | P | 414.8 | P |
| | | gi = 2104954/ug = Mm 19127/len = 977/STRA = for | | | | | | |
| 11 | 93539_at | Cluster Incl AI118854: uc14105.r1 Mus musculus cDNA, 5 end/clone = IMAGE-1397985/clone_end = 5/gb = AI118854/ | 486.9 | P | 301.4 | P | 315.7 | P |
| | | gi = 3519178/ug = Mm.2000/len = 281/STRA = for | | | | | | |
| 11 | 93575_at | Cluster Incl AF051102: Mus musculus gamma glutamyl hydrolase precursor, mRNA, complete cds/cds = (90,1043)/ | 621.8 | P | 369.4 | P | 305.8 | P |
| | | gb = AF051102/gi = 3831561/ug = Mm 20461/len = 1295/STRA = for | | | | | | |
| 11 | 93812_at | Cluster Incl U53455 Mus musculus chloride ion current inducer protein (GLCI) mRNA, partial cds/cds = (0,710)/gb = U53455/ | 855.2 | P | 322.3 | P | 323.3 | P |
| | | gi = 1399647/ug = Mm.21482/len = 711/STRA = for | | | | | | |
| 11 | 93830_at | Cluster Incl AI851199 UI-M-BH0-ajx-e-10-0-UI s1 Mus musculus cDNA, 3 end/clone = UI-M-BH0-ajx-e-10-0-UI/ | 2841.5 | P | 2455.5 | P | 2187.8 | P |
| | | clone_end = 3/gb = AI851199/gi = 5495105/ug = Mm.21559/len = 301/STRA = for | | | | | | |
| 11 | 93839_at | Cluster Incl AI854888 UI-M-BH0-akb-g-05-0-UI.s1 Mus musculus cDNA, 3 end/clone = UI-M-BH0-akb-g-05-0-UI/ | 860.7 | P | 879.7 | P | 655 | P |
| | | clone_end = 3/gb = AI854888/gi = 5498794/ug = Mm 21606/len = 468/STRA = for | | | | | | |
| 11 | 93984_at | Cluster Incl AF002718 Mus musculus ATPase inhibitor (IF1) mRNA, complete cds/cds = (30,350)/gb = AF002718/ | 2932.1 | P | 2226.7 | P | 2363.2 | P |
| | | gi = 2367440/ug = Mm 2171/len = 509/STRA = for | | | | | | |
| 11 | 94032_at | Cluster Incl AI845103 UI-M-BG0-ahs-a-01-0-UI s1 Mus musculus cDNA, 3 end/clone = UI-M-BG0-ahs-a-01-0-UI/ | 1280.7 | P | 627.8 | P | 768.5 | P |
| | | clone_end = 3/gb = AI845103/gi = 5489009/ug = Mm 21905/len = 309/STRA = for | | | | | | |
| 11 | 94038_at | Cluster Incl AA675468: vr72c04 s1 Mus musculus cDNA, 5 end/clone = IMAGE-1134246/clone_end = 5/gb = AA675468/ | 1358.8 | P | 881.8 | P | 1151.4 | P |
| | | gi = 2652705/ug = Mm.21932/len = 540/STRA = for | | | | | | |
| 11 | 94047_at | Cluster Incl AW122935 UI-M-BH2.1-apa-h-12-0-UI s1 Mus musculus cDNA, 3 end/clone = UI-M-BH2.1-apa-h-12-0-UI/ | 726 | P | 657.6 | P | 709.9 | P |
| | | clone_end = 3/gb = AW122935/gi = 6098465/ug = Mm 21976/len = 365/STRA = for | | | | | | |
| 11 | 94206_at | Cluster Incl AC002397 Mouse chromosome 6 Bac-284H12 (Research Genetics mouse BAC library) complete sequence/ | 1100.6 | P | 850.9 | P | 1168.6 | P |
| | | cds = (108,488)/gb = AC002397/gi = 3287367/ug = Mm 22195/len = 568/STRA = for | | | | | | |
| 11 | 94210_at | Cluster Incl AW121333 UI-M-BH2.2-aom-b-04-0-UI.s1 Mus musculus cDNA, 3 end/clone = UI-M-BH2.2-aom-b-04-0-UI/ | 1101 | P | 958.6 | P | 1096.7 | P |
| | | clone_end = 3/gb = AW121333/gi = 6096666/ug = Mm 22201/len = 494/STRA = for | | | | | | |
| 11 | 94229_at | Cluster Incl AW124489: UI-M-BH2.1-apo-a-01-0-UI s1 Mus musculus cDNA, 3 end/clone = UI-M-BH2.1-apo-a-01-0-UI/ | 671.3 | P | 540.6 | P | 573.8 | P |
| | | clone_end = 3/gb = AW124489/gi = 6099984/ug = Mm 22281/len = 513/STRA = for | | | | | | |
| 11 | 94252_at | Cluster Incl AJ006587 Mus musculus mRNA for translation initiation factor eIF2 gamma X/cds = (8,1426)/gb = AJ006587/ | 2774.2 | P | 1246.9 | P | 2115.5 | P |
| | | gi = 3790183/ug = Mm 22387/len = 1787/STRA = for | | | | | | |
| 11 | 94266_at | Cluster Incl AW121737: UI-M-BH2-3-aoe-b-10-0-UI s1 Mus musculus cDNA, 3 end/clone = UI-M-BH2-3-aoe-b-10-0-UI/ | 2024.6 | P | 1012.9 | P | 1191 | P |
| | | clone_end = 3/gb = AW121737/gi = 6097070/ug = Mm 22469/len = 365/STRA = for | | | | | | |
| 11 | 94811_s_at | Cluster Incl AJ002366 Mus musculus mRNA for transcription factor TFIIH, 62 kD subunit/cds = (44,1687)/gb = AJ002266/ | 357 | P | 47.6 | P | 52.3 | P |
| | | gi = 2582796/ug = Mm 22700/len = 2471/STRA = for | | | | | | |
| 11 | 94817_at | Cluster Incl X60676 M musculus HSP47 mRNA/cds = (256,1509)/gb = X60676/gi = 51449/ug = Mm 22708/len = 2273/ | 4178.1 | P | 1526.4 | P | 1907.1 | P |
| | | STRA = for | | | | | | |
| 11 | 94820_r_at | Cluster Incl AF0058886 Mus musculus cyclin I mRNA, complete cds/cds = (286,1419)/gb = AF005886/gi = 4101686/ | 1073.9 | P | 796.5 | P | 738.4 | P |
| | | ug = Mm 22711/len = 1548/STRA = for | | | | | | |
| 11 | 94915_at | Cluster Incl X58990 Mouse mCyp-S1 mRNA for cyclophilin CyP-S1/cds = (166,792)/gb = X58990/gi = 53034/ug = Mm 2412/ | 2124.5 | P | 1522.1 | P | 1800.3 | P |
| | | len = 998/STRA = for | | | | | | |
| 11 | 95430_f_at | Cluster Incl AI854154 UI-M-BH0-ajw-h-03-0-UI.s1 Mus musculus cDNA, 3 end/clone = UI-M-BH0-ajw-h-03-0-UI/ | 837.5 | P | 496.7 | P | 483.7 | P |
| | | clone_end = 3/gb = AI854154/gi = 5498060/ug = Mm 24460/len = 561/STRA = for | | | | | | |
| 11 | 95493_at | Cluster Incl X66405 Procollagen, type VI, alpha 1/cds = (131,3208)/gb = X66405/gi = 50478/ug = Mm 2509/len = 3991/ | 3749.6 | P | 2758.3 | P | 3109.2 | P |
| | | STRA = for | | | | | | |
| 11 | 96057_at | Cluster Incl AI647493 uk42h02.x1 Mus musculus cDNA, 3 end/clone = IMAGE-1971699/clone_end = 3/gb = AI647493/ | 629.3 | P | 423 | P | 478 | P |
| | | gi = 4726171/ug = Mm 2621/len = 761/STRA = for | | | | | | |
| 11 | 96058_s_at | Cluster Incl U07235 Mus musculus aldehyde dehydrogenase (ALDH2) mRNA, nuclear gene encoding mitochondrial | 414.3 | P | 93.6 | P | 248 | P |
| | | protein, complete cds/cds = (105,1664)/gb = U07235/gi = 466253/ug = Mm 2621/len = 1954/STRA = for | | | | | | |
| 11 | 96059_at | Cluster Incl AA823336 vp35g10 r1 Mus musculus cDNA, 5 end/clone = IMAGE-1078722/clone_end = 5/gb = AA823336/ | 389.2 | P | 231.1 | P | 239.5 | P |
| | | gi = 2893204/ug = Mm 26217/len = 366/STRA = for | | | | | | |
| 11 | 96086_at | Cluster Incl AW122530 UI-M-BH2.2-aox-b-12-0-UI s1 Mus musculus cDNA, 3 end/clone = UI-M-BH2.2-aox-b-12-0-UI/ | 482.2 | P | 385 | P | 334.6 | P |
| | | clone_end = 3/gb = AW122530/gi = 6098054/ug = Mm 26680/len = 206/STRA = for | | | | | | |
| 11 | 96115_at | Cluster Incl U28168: Adenomatosis polyposis coli/cds = (35,592)/gb = U28168/gi = 1142715/ug = Mm 2698/len = 719/ | 2251.7 | P | 2092.3 | P | 2382 | P |
| | | STRA = for | | | | | | |
| 11 | 96617_at | Cluster Incl AI844737 UI-M-AL1-ahq-c-11-0-UI s1 Mus musculus cDNA, 3 end/clone = UI-M-AL1-ahq-c-11-0-UI/ | 597.8 | P | 416.5 | P | 475.9 | P |
| | | clone_end = 3/gb = AI844737/gi = 5488643/ug = Mm 27128/len = 178/STRA = for | | | | | | |
| 11 | 96652_at | Cluster Incl AI849911: UI-M-BG0-ahz-d-03-0-UI s1 Mus musculus cDNA, 3 end/clone = UI-M-BG0-ahz-d-03-0-UI/ | 610.7 | P | 389.4 | P | 561 | P |
| | | clone_end = 3/gb = AI849911/gi = 5493817/ug = Mm 27332/len = 385/STRA = for | | | | | | |
| 11 | 96661_at | Cluster Incl AA734806 vp38b03 r1 Mus musculus cDNA, 5 end/clone = IMAGE-1078925/clone_end = 5/gb = AA734806/ | 1178 | P | 736.7 | P | 789.7 | P |
| | | gi = 2756473/ug = Mm.27357/len = 446/STRA = for | | | | | | |
| 11 | 96667_at | Cluster Incl AI851542 UI-M-BH0-aki-d-01-0-UI.s1 Mus musculus cDNA, 3 end/clone = UI-M-BH0-aki-d-01-0-UI/ | 427.1 | P | 238.5 | P | 193.5 | P |
| | | clone_end = 3/gb = AI851542/gi = 5495448/ug = Mm.27389/len = 477/STRA = for | | | | | | |
| 11 | 96668_at | Cluster Incl AI845867 UI-M-AO1-aeg-g-01-0-UI s1 Mus musculus cDNA, 3 end/clone = UI-M-AO1-aeg-g-01-0-UI/ | 1088.8 | P | 916.7 | P | 835.2 | P |
| | | clone_end = 3/gb = AI845867/gi = 5489773/ug = Mm 27393/len = 345/STRA = for | | | | | | |
| 11 | 98710_at | Cluster Incl AI854262 UI-M-BH0-aja-b-11-0-UI s1 Mus musculus cDNA, 3 end/clone = UI-M-BH0-aja-b-11-0-UI/ | 1969.2 | P | 767.8 | P | 940.4 | P |
| | | clone_end = 3/gb = AI854262/gi = 5498168/ug = Mm 27624/len = 547/STRA = for | | | | | | |
| 11 | 97268_i_at | Cluster Incl AI848699 UI-M-AH1-agv-g-05-0-UI.s1 Mus musculus cDNA, 3 end/clone = UI-M-AH1-agv-g-05-0-UI/ | 1083.5 | P | 984.7 | P | 951.8 | P |
| | | clone_end = 3/gb = AI848699/gi = 5492600/ug = Mm 27904/len = 531/STRA = for | | | | | | |
| 11 | 97269_f_at | Cluster Incl AI848699.UI-M-AH1-agv-g-05-0-UI.s1 Mus musculus cDNA, 3 end/clone = UI-M-AH1-agv-g-05-0-UI/ | 1041.8 | P | 919.6 | P | 1220.4 | P |
| | | clone_end = 3/gb = AI848699/gi = 5492600/ug = Mm.27904/len = 531/STRA = for | | | | | | |
| 11 | 97797_at | Cluster Incl AW123142.UI-M-BH2.1-apd-b-05-0-UI s1 Mus musculus cDNA, 3 end/clone = UI-M-BH2.1-apd-b-05-0-UI/ | 854.9 | P | 714.7 | P | 600.4 | P |
| | | clone_end = 3/gb = AW123142/gi = 6098672/ug = Mm 28111/len = 572/STRA = for | | | | | | |
| 11 | 97820_at | Cluster Incl AB027012 Mus musculus galK mRNA for galactokinase, complete cds/cds = (0,1175)/gb = AB027012/ | 414.4 | P | 147.9 | P | 239.1 | P |
| | | gi = 5931607/ug = Mm.2820/len = 1293/STRA = for | | | | | | |
| 11 | 97828_at | Cluster Incl AF033115 Mus musculus proapoptotic protein (Siva) gene, complete cds/cds = (0,527)/gb = AF0033115/ | 661 | P | 425.3 | P | 404.6 | P |
| | | gi = 2921301/ug = Mm 28216/len = 528/STRA = for | | | | | | |
| 11 | 97863_at | Cluster Incl AW125274: UI-M-BH2.1-aps-h-08-0-UI.s1 Mus musculus cDNA, 3 end/clone = UI-M-BH2.1-aps-h-08-0-UI/ | 410.9 | P | 421.6 | P | 383.4 | P |
| | | clone_end = 3/gb = AW125274/gi = 6100804/ug = Mm.28326/len = 148/STRA = for | | | | | | |
| 11 | 97866_at | Cluster Incl AI842858: UI-M-AN1-1-afb-c-09-0-UI.s1 Mus musculus cDNA, 3 end/clone = UI-M-AN1-afb-c-09-0-UI/ | 440.3 | P | 373.9 | P | 303.2 | P |
| | | clone_end = 3/gb = AI842858/gi = 5477071/ug = Mm 28327/len = 254/STRA = for | | | | | | |
| 11 | 97909_at | Cluster Incl AI838080 UI-M-AL0-abv-e-12-0-UI.s1 Mus musculus cDNA, 3 end/clone = UI-M-AL0-abv-e-12-0-UI/ | 2852.8 | P | 1505.3 | P | 1130.2 | P |
| | | clone_end = 3/gb = AI838080/gi = 5472293/ug = Mm.28479/len = 226/STRA = for | | | | | | |
| 11 | 98953_at | Cluster Incl AW048032 UI-M-BH1-alv-b-08-0-UI s1 Mus musculus cDNA, 3 end/clone = UI-M-BH1-alv-b-08-0-UI/ | 619.1 | P | 475.2 | P | 467.5 | P |
| | | clone_end = 3/gb = AW048032/gi = 5908526/ug = Mm.28753/len = 587/STRA = for | | | | | | |
| 11 | 94499_at | Cluster Incl AI835427 UI-M-AQ0-aaj-f-10-0-UI s1 Mus musculus cDNA, 3 end/clone = UI-M-AQ0-aaj-f-10-0-UI/ | 702.8 | P | 493.1 | P | 256.4 | P |
| | | clone_end = 3/gb = AI835427/gi = 5469640/ug = Mm 28938/len = 236/STRA = for | | | | | | |
| 11 | 94552_at | Cluster Incl AI839477 UI-M-AL0-abu-e-11-0-UI s2 Mus musculus cDNA, 3 end/clone = UI-M-AL0-abu-e-11-0-UI/ | 3287.9 | P | 3078.4 | P | 2984.3 | P |
| | | clone_end = 3/gb = AI839477/gi = 54773690/ug = Mm 29089/len = 474/STRA = for | | | | | | |
| 11 | 94558_g_at | Cluster Incl AI850893 UI-M-BH0-ajt-g-07-0-UI s1 Mus musculus cDNA, 3 end/clone = UI-M-BH0-ajt-g-07-0-UI/clone_end = 3/ | 928.6 | P | 394.9 | P | 383.8 | P |
| | | gb = AI850893/gi = 5494799/ug = Mm.29105/len = 375/STRA = for | | | | | | |
| 11 | 95053_s_at | Cluster Incl AA674669 vq02d04.r1 Mus musculus cDNA, 5 end/clone = IMAGE-1093063/clone_end = 5/gb = AA674669/ | 1778 | P | 1392.1 | P | 1620.4 | P |
| | | gi = 2651906/ug = Mm 29141/len = 414/STRA = for | | | | | | |
| 11 | 95076_at | Cluster Incl AI848476 UI-M-AP1-agf-a-07-0-UI s2 Mus musculus cDNA, 3 end/clone = UI-M-AP1-agf-a-07-0-UI/ | 1822 | P | 1485.2 | P | 1571.7 | P |
| | | clone_end = 3/gb = AI848476/gi = 5492382/ug = Mm.29202/len = 304/STRA = for | | | | | | |
| 11 | 95090_at | Cluster Incl AA688600 vu18f09 r1 Mus musculus cDNA, 5 end/clone = IMAGE-1181033/clone_end = 5/gb = AA688600/ | 1265.1 | P | 1136.9 | P | 1082.7 | P |
| | | gi = 2678029/ug = Mm 29269/len = 362/STRA = rev | | | | | | |
| 11 | 95100_at | Cluster Incl AI847314 UI-M-AN1-afh-e-04-0-UI s1 Mus musculus cDNA, 3 end/clone = UI-M-AN1-afh-e-04-0-UI/ | 2340 | P | 1913 | P | 1840.3 | P |
| | | clone_end = 3/gb = AI847314/gi = 5491220/ug = Mm 29341/len = 379/STRA = for | | | | | | |
| 11 | 95120_at | Cluster Incl AI837621. UI-M-AK0-adg-d-09-0-UI.s1 Mus musculus cDNA, 3 end/clone = UI-M-AK0-adg-d-09-0-UI/ | 346.9 | P | 150.7 | P | 226.3 | P |
| | | clone_end = 3/gb = AI837621/gi = 5471834/ug = Mm 29386/len = 473/STRA = for | | | | | | |
| 11 | 95652_at | Cluster Incl AF110520 Mus musculus major histocompatibility complex region NG27, NG28, RPS28, NADH | 3144.7 | P | 1736.5 | P | 2670.3 | P |
| | | oxidoreductase, NG29, KIFC1, Fas-binding protein, BING1, tapasin, RalGDS-like, KE2, BING4, beta 1,3-galactosyl | | | | | | |
| | | transferase, and RPS18 genes, complete cds, S/STRA = for | | | | | | |
| 11 | 95662_at | Cluster Incl AW211667 uo83c04 y1 Mus musculus cDNA, 5 end/clone = IMAGE-2649126/clone_end = 5/gb = AW211667/ | 292.5 | P | 153 | P | 218.2 | P |
| | | gi = 6517563/ug = Mm 29562/len = 454/STRA = for | | | | | | |
| 11 | 95674_r_at | Cluster Incl AI851985 UI-M-BH0-aiq-d-08-0-UI s1 Mus musculus cDNA, 3 end/clone = UI-M-BH0-aiq-d-08-0-UI/ | 2133.9 | P | 1274.1 | P | 1575.6 | P |
| | | clone_end = 3/gb = AI851985/gi = 5495891/ug = Mm 29586/len = 404/STRA = for | | | | | | |
| 11 | 95689_at | Cluster Incl AI840995 UI-M-AK0-adk-g-06-0-UI s1 Mus musculus cDNA, 3 end/clone = UI-M-AK0-adk-g-06-0-UI/ | 1225.6 | P | 723.4 | P | 863.5 | P |
| | | clone_end = 3/gb = AI840995/gi = 5475208/ug = Mm 29650/len = 438/STRA = for | | | | | | |
| 11 | 95715_at | Cluster Incl AW125739 UI-M-BH2 2-aqi-g-05-0-UI s1 Mus musculus cDNA, 3 end/clone = UI-M-BH2-agi-g-05-0-UI/ | 2643.7 | P | 1751.2 | P | 2314 | P |
| | | clone_end = 3/gb = AW125739/gi = 6101234/ug = Mm 29714/len = 321/STRA = for | | | | | | |
| 11 | 96272_at | Cluster Incl AI848471 UI-M-AH1-ags-h-12-0-UI s1 Mus musculus cDNA, 3 end/clone = UI-M-AH1-ags-h-12-0-UI/ | 799.2 | P | 785.4 | P | 727.2 | P |
| | | clone_end = 3/gb = AI848471/gi = 5492377/ug = Mm 29855/len = 427/STRA = for | | | | | | |
| 11 | 96284_at | Cluster Incl AW121446 UI-M-BH2.2-aon-d-06-0-UI s1 Mus musculus cDNA, 3 end/clone = UI-M-BH2 2-aon-d-06-0-UI/ | 922.4 | P | 777 | P | 848.4 | P |
| | | clone_end = 3/gb = AW121446/gi = 6096779/ug = Mm 29873/len = 220/STRA = for | | | | | | |
| 11 | 96285_at | Cluster Incl AJ001616 Mus musculus mRNA for myeloid associated differentiation protein/cds = (187,1077)/gb = AJ001616/ | 2558.8 | P | 1659.8 | P | 1300.7 | P |
| | | gi = 2463264/ug = Mm 29874/len = 2822/STRA = for | | | | | | |
| 11 | 96320_at | Cluster Incl AW121862 UI-M-BH2.3-aoi-c-03-0-UI.s1 Mus musculus cDNA, 3 end/clone = UI-M-BH2-aoi-c-03-0-UI/ | 687.8 | P | 553.4 | P | 566.3 | P |
| | | clone_end = 3/gb = AW121862/gi = 6097236/ug = Mm 29937/len = 495/STRA = for | | | | | | |
| 11 | 96345_at | Cluster Incl AI837012 UI-M-AJ0-aaz-h-08-0-UI s1 Mus musculus cDNA, 3 end/clone = UI-M-AJ0-aaz-h-08-0-UI/ | 683.1 | P | 405.9 | P | 563 | P |
| | | clone_end = 3/gb = AI837012/gi = 5471225/ug = Mm 29995/len = 460/STRA = for | | | | | | |
| 11 | 96346_at | Cluster Incl AI854020 UI-M-BH0-ajm-b-01-0-UI s1 Mus musculus cDNA, 3 end/clone = UI-M-BH0-ajm-b-01-0-UI/ | 748.9 | P | 403 | P | 679.2 | P |
| | | clone_end = 3/gb = AI854020/gi = 5497926/ug = Mm 29996/len = 262/STRA = for | | | | | | |
| 11 | 96869_at | Cluster Incl AW124839 UI-M-BH2.1-apk-f-02-0-UI s1 Mus musculus cDNA, 3 end/clone = UI-M-BH2 1-apk-f-02-0-UI/ | 3281.1 | P | 2631.4 | P | 2853.3 | P |
| | | clone_end = 3/gb = AW124839/gi = 6100369/ug = Mm 30064/len = 465/STRA = for | | | | | | |
| 11 | 96887_at | Cluster Incl Y08702 M musculus mRNA for neuronal protein 15.6/cds = (51,452)/gb = Y08702/gi = 1771305/Mm.30084/ | 1865.1 | P | 1162.3 | P | 1507.9 | P |
| | | len = 554/STRA = for | | | | | | |
| 11 | 96888_at | Cluster Incl AI839814 UI-M-AN0-acq-b-07-0-UI s1 Mus musculus cDNA, 3 end/clone = UI-M-AN0-acq-b-07-0-UI/ | 3464.4 | P | 2878.3 | P | 3467 | P |
| | | clone_end = 3/gb = AI839814/gi = 5473992/ug = Mm 30085/len = 342/STRA = for | | | | | | |
| 11 | 96890_at | Cluster Incl AW060971 UI-M-BH1-anv-h-09-0-UI s1 Mus musculus cDNA, 3 end/clone = UI-M-BH1-anv-h-09-0-UI/ | 362.4 | P | 242.9 | P | 212.3 | P |
| | | clone_end = 3/gb = AW060971/gi = 6008722/ug = Mm 30092/len = 539/STRA = for | | | | | | |
| 11 | 96911_at | Cluster Incl U34960 Mouse G protein beta 2 subunit mRNA, complete cds/cds = (0,1022)/gb = U34960/gi = 984550/ | 2467.6 | P | 1785 | P | 1603.8 | P |
| | | ug = Mm 30141/len = 1023/STRA = for | | | | | | |
| 11 | 96915_f_at | Cluster Incl AA879764 vv98d04 r1 Mus musculus cDNA, 5 end/clone = IMAGE-1230439/clone_end = 5/gb = AA879764/ | 1622.3 | P | 1078.8 | P | 1216.7 | P |
| | | gi = 2988747/ug = Mm 30150/len = 357/STRA = for | | | | | | |
| 11 | 96948_at | Cluster Incl AI845337 UI-M-BG0-ahx-b-12-0-UI.s1 Mus musculus cDNA, 3 end/clone = UI-M-BG0-ahx-b-12-0-UI/ | 817 | P | 607.5 | P | 609.4 | P |
| | | clone_end = 3/gb = AI845337/gi = 5489243/ug = Mm 30204/len = 210/STRA = for | | | | | | |
| 11 | 97446_at | Cluster Incl AW125010 UI-M-BH2.1-apv-g-03-0-UI s1 Mus musculus cDNA, 3 end/clone = UI-M-BH2 1-apv-g-03-0-UI/ | 3107.7 | P | 250.7 | P | 230.9 | P |
| | | clone_end = 3/gb = AW125010/gi = 6100540/ug = Mm 30243/len = 428/STRA = for | | | | | | |
| 11 | 97452_at | Cluster Incl AI853078 UI-M-BH0-ajf-a-06-0-UI.s1 Mus musculus cDNA, 3 end/clone = UI-M-BH0-ajf-a-06-0-UI/ | 886.5 | P | 645.8 | P | 818.8 | P |
| | | clone_end = 3/gb = AI853078/gi = 5496984/ug = Mm 30255/len = 429/STRA = rev | | | | | | |
| 11 | 97464_at | Cluster Incl AI844416 UI-M-AL1-ahp-g-01-0-UI s1 Mus musculus cDNA, 3 end/clone = UI-M-AL1-ahp-g-01-0-UI/ | 1351.6 | P | 842.5 | P | 765.3 | P |
| | | clone_end = 3/gb = AI844416/gi = 5488322/ug = Mm 3035/len = 432/STRA = for | | | | | | |
| 11 | 98120_at | Cluster Incl AI844807 UI-M-AH1-agx-b-03-0-UI s1 Mus musculus cDNA, 3 end/clone = UI-M-AH1-agx-b-03-0-UI/ | 688.6 | P | 492.7 | P | 493 | P |
| | | clone_end = 3/gb = AI844807/gi = 5488713/ug = Mm 34951/len = 517/STRA = for | | | | | | |
| 11 | 98147_at | Cluster Incl AC002397: Mouse chromosome 6 BAC-284H12 (Research Genetics mouse BAC library) complete sequence/ | 1028.3 | P | 829 | P | 639.8 | P |
| | | cds = (52,2628)/gb = AC002397/gi = 3287367/ug = Mm 3571/len = 3176/STRA = rev | | | | | | |
| 11 | 98149_s_at | Cluster Incl AW046496: UI-M-BH1-akz-d-01-0-UI.s1 Mus musculus cDNA, 3 end/clone = UI-M-BH1-akz-d-01-0-UI/ | 388.7 | P | 336 | P | 342.2 | P |
| | | clone _end = 3/gb = AW046496/gi = 5907025/ug = Mm.3572/len = 413/STRA = for | | | | | | |
| 11 | 98516_at | Cluster Incl AI853136: UI-M-BH0-ajf-f-11-0-UI s1 Mus musculus cDNA, 3 end/clone = UI-M-BH0-ajf-f-11-0-UI/clone_end = 3/ | 1030.3 | P | 833 | P | 856.7 | P |
| | | gb = AI853136/gi = 5497042/ug = Mm.35790/len = 291/STRA = for | | | | | | |
| 11 | 98535_at | Cluster Incl AF076156 Calechol-O-methyltransferase/cds = (163,960)/gb = AF076156/gi = 3493252/ug = Mm 35853/ | 814.6 | P | 440 | P | 659.7 | P |
| | | len = 1255/STRA = for | | | | | | |
| 11 | 98544_at | Cluster Incl U53514 Mus musculus guanylate kinase (gmk) mRNA, complete cds/cds = (24,620)/gb = U53514/gi = 1408589/ | 588.7 | P | 351.7 | P | 331.7 | P |
| | | ug = Mm 3624/len = 663/STRA = for | | | | | | |
| 11 | 98627_at | Cluster Incl X81580: Insulin-like growth factor binding protein 2/cds = (71,988)/gb = X81580 gi = 550378/ug = Mm 38748/ | 606.8 | P | 274.3 | P | 161.8 | P |
| | | len = 1310/STRA = for | | | | | | |
| 11 | 99115_at | Cluster Incl AW046350: UI-M-BH1-ali-a-01-0-UI s1 Mus musculus cDNA, 3 end/clone = UI-M-BH1-ali-a-01-0-UI/ | 2580.8 | P | 1791.7 | P | 2725 | P |
| | | clone_end = 3/gb = AW046350/gi = 5906879/ug = Mm 400/len = 253/STRA = for | | | | | | |
| 11 | 99164_at | Cluster Incl AI844560 UI-M-AJ1-ahg-c-05-0-UI s2 Mus musculus cDNA, 3 end/clone = UI-M-AJ1-ahg-c-05-0-UI/ | 436.8 | P | 379.6 | P | 296 | P |
| | | clone_end = 3/gb = AI844560/gi = 5488466/ug = Mm 41680/len = 357/STRA = for | | | | | | |
| 11 | 99175_at | Cluster Incl AI843396 UI-M-AQ1-adz-g-07-0-UI s1 Mus musculus cDNA, 3 end/clone = UI -M-AQ1-adz-g-07-0-UI/ | 805.1 | P | 353.6 | P | 484.2 | P |
| | | clone_end = 3/gb = AI843396/gi = 5477609/ug = Mm 41780/len = 289/STRA = for | | | | | | |
| 11 | 99584-at | Cluster Incl D14883 CD82 anligen/cds = (102,902)/gb = D14883/gi = 984036/ug = Mm 4261/len = 1657/STRA = for | 531.3 | P | 509.2 | P | 403.8 | P |
| 11 | 99599_s_at | Cluster Incl AW210320: ul53e09 y1 Mus musculus cDNA, 5 end/clone = IMAGE-2102056/clone_end = 5/gb = AW210320/ | 2169.9 | P | 1241.5 | P | 1710.5 | P |
| | | gi = 6516260/ug = Mm 42855/len = 477/STRA = for | | | | | | |
| 11 | 99629_at | Cluster Incl AW060710: UI-M-BH1-anl-g-04-0-UI s1 Mus musculus cDNA, 3 end/clone = UI -M-BH1-anl-g-04-0-UI/ | 1164.8 | P | 963.2 | P | 882.4 | P |
| | | clone_end = 3/gb = AW060710/gi = 6008461/ug = Mm 4337/len = 535/STRA = for | | | | | | |
| 11 | 99651_at | Cluster Incl AI849549 UI-M-AH1-agr-a-10-0-UI s1 Mus musculus cDNA, 3 end/clone = UI-M-AH1-agr-a-10-0-UI/ | 486.3 | P | 472.8 | P | 353.6 | P |
| | | clone end = 3/gb = AI849549/gi = 5493455/ug = Mm 43737/len = 279/STRA = for | | | | | | |
| 11 | 99665_at | Cluster Incl U05252 Mus musculus nuclear matrix attachment DNA-binding protein SATB1 mRNA, complete cds/ | 694.8 | P | 368.5 | P | 606.8 | P |
| | | cds = (198,2492)/gb = U05252/gi = 453370/ug = Mm 4381/len = 2922/STRA = for | | | | | | |
| 11 | 100039_at | Cluster Incl AW125880 UI-M-BH2 2-aqk-g-03-0-UI s1 Mus musculus cDNA, 3 end/clone = UI-M-BH2 2-aqk-g-03-0-UI/ | 591.4 | P | 161.2 | P | 304.8 | P |
| | | clone_end = 3/gb = AW125880/gi = 6101410/ug = Mm 44220/len = 490/STRA = for | | | | | | |
| 11 | 100153_at | Cluster Incl X15052 Neural cell adhesion molecule/cds = (0,1246)/gb = X15052/gi = 53349/ug = Mm 4974/len = 4552/ | 744.5 | P | 489.9 | P | 406.2 | P |
| | | STRA = for | | | | | | |
| 11 | 100538_at | Cluster Incl M35725 Mouse Cu-Zn superoxide dismutase mRNA, complete cds/cds(4,468)/gb = M35725/gi = 192929/ | 1671.9 | P | 784.3 | P | 1542.2 | P |
| | | ug = Mm 5274/len = 469/STRA = for | | | | | | |
| 11 | 100540_at | Cluster Incl M63848: Mouse leukotnene A-4 hydrolase mRNA, complete cds/cds = (80,1915)/gb = M63848/gi = 198883/ | 904.5 | P | 720.7 | P | 816.6 | P |
| | | ug-Mm 533/len = 2039/STRA = for | | | | | | |
| 11 | 100576_at | Cluster Incl U57746: Mus musculus platelet-activating, factor acetylhydrolase isoform lb gamma subunit (PAF-AH gamma) | 666.5 | P | 354.6 | P | 439.2 | P |
| | | mRNA, complete cds/cds = (129,627)/gb = U57746/gi = 1373360/ug = Mm 597/len = 872/STRA = for | | | | | | |
| 11 | 100629_at | Cluster Incl U24428 Mus musculus mu-class glutathione s-transferase (mGSTM5) mRNA, complete cds/cds = (96,770)/ | 533.5 | P | 307.8 | P | 408 | P |
| | | gb = U24428/gi = 1002385/ug = Mm.667/len = 907/STRA = for | | | | | | |
| 11 | 101000_at | Cluster Incl D78643 Mouse seizure-related mRNA, partial cds/cds = (0,422)/gb = D78643/gi = 1304167/ug = Mm 675/ | 664.6 | P | 492.3 | P | 358.4 | P |
| | | len = 1492/STRA = for | | | | | | |
| 11 | 101007_at | Cluster Incl AI845732 UI-M-AQ1-aeb-b-04-0-UI s1 Mus musculus cDNA, 3 end/clone = UI-M-AQ1-aeb-b-04-0-UI/ | 1191 | P | 960.3 | P | 1120.2 | P |
| | | clone_end = 3/gb = AI845732/gi = 5489638/ug = Mm 6797/len = 184/STRA = for | | | | | | |
| 11 | 101013_at | Cluster Incl U52623 Mus musculus omithine decarboxylase anlizyme gene, complete cds/cds = (0,683)/gb = U52823/ | 4214.6 | P | 3327.3 | P | 3883.9 | P |
| | | gi = 1924975/ug = Mm 683/len = 684/STRA = for | | | | | | |
| 11 | 101042_f_at | Cluster Incl U51014 Peptidase 4/cds = (54,1535)/gb = U51014/gi = 1236705/ug = Mm 69751/len = 1876/STRA = for | 473.6 | P | 319.8 | P | 324.7 | P |
| 11 | 101053_at | Cluster Incl AW046847: UI-M-BH1-alz-h-03-0-UI s1 Mus musculus cDNA, 3 end/clone = UI-M-BH1-alz-h-03-0-UI/ | 589.3 | P | 575.6 | P | 603.9 | P |
| | | clone_end = 3/gb = AW046847/gi = 5907376/ug = Mm 7039/len = 242/STRA = for | | | | | | |
| 11 | 101059_at | Cluster Incl D76440 Necdin/cds = (86,1063)/gb = D76440/gi = 1040690/ug = Mm 7089/len = 1608/STRA = for | 822 | P | 278.2 | P | 405.5 | P |
| 11 | 101072_at | Cluster Incl AW124250 UI-M-BH2.1-aph-f-11-0-UI s1 Mus musculus cDNA, 3 end/clone = UI-M-BH2.1-aph-f-11-0-UI/ | 2474.9 | P | 2053.5 | P | 2202.2 | P |
| | | clone_end = 3/gb = AW124250/gi = 6099780/ug = Mm 7215/len = 267/STRA = for | | | | | | |
| 11 | 101080_at | Cluster Incl AB009993 Mus musculus mRNA for collagen a1(V), complete cds/cds = (131,5647)/gb = AB009993/ | 542.5 | P | 158.8 | P | 190.8 | P |
| | | gi = 3219171/ug = Mm 7281/len = 6156/STRA = for | | | | | | |
| 11 | 101495_at | Cluster Incl X59047 M musculus MD3 mRNA/cds = UNKNOWN/gb = X59047/gi = 53037/ug = Mm 806/len = 1234/STRA = for | 1397.1 | P | 995.5 | P | 1148 | P |
| 11 | 101589_at | Cluster Incl X12944 Mouse mRNA for HMG-17 chromosomal protein/cds = (39,311)/gb = X12944/gi = 51336/ug = Mm 911/ | 2288.5 | P | 1806.4 | P | 914.8 | P |
| | | len = 1113/STRA = for | | | | | | |
| 11 | 101976_at | Cluster Incl AF071314: Mus musculus COP9 complex subunit 4 (COPS4) mRNA, complete cds/cds = (37,1257)/ | 424.2 | P | 365.2 | P | 393.7 | P |
| | | gb = AF071314/gi = 3309169/ug = Mm 957/len = 1620/STRA = for | | | | | | |
| 11 | 102003_at | Cluster Incl AI842159 UI-M-AN1-afj-f-10-0-UI.s1 Mus musculus cDNA, 3 end/clone = UI-M-AN1-afj-f-10-0-UI/clone_end = 3/ | 466.6 | P | 400.2 | P | 326.1 | P |
| | | gb = AI842159/gi = 5476372/ug = Mm 99132/len = 277/STRA = for | | | | | | |
| 11 | 103332_at | Cluster Incl AI197507 ud56b04 r1 Mus musculus cDNA, 5 end/clone = IMAGE-1449871/clone_end = 5/gb = AI197507/ | 119.1 | P | 101.6 | P | 58.5 | P |
| | | gi = 3750113/ug = Mm 18062/len = 436/STRA = for | | | | | | |
| 11 | 103591_at | Cluster Incl D49439 Mouse mRNA for TFIID subunit p80, complete cds/cds = (10,2046)/gb = D49439/gi = 1372970/ | 284 | P | 199.8 | P | 202 | P |
| | | ug = Mm 1994/len = 2192/STRA = for | | | | | | |
| 11 | 103642_at | Cluster Incl AB001927: Mus musculus mRNA for ras-GTPase-activating protein SH3-domain binding protein, complete cds/ | 1602.5 | P | 1536.7 | P | 1259.6 | P |
| | | cds = (68,1465)/gb = AB001927/gi = 1902906/Ug = Mm.2038/len = 2686/STRA = for | | | | | | |
| 11 | 103656_at | Cluster Incl Y16518 Mus musculus mRNA for G protein-coupled receptor, P40GPRT/cds = (257,1456)/gb = Y16518/ | 822.9 | P | 432.7 | P | 614.4 | P |
| | | gi = 3492792/ug = Mm 20522/len = 1581/STRA = for | | | | | | |
| 11 | 103817_at | Cluster Incl AJ006469 Mus musculus mRNA for cartilage-associated protein (CASP)/cds = (71,1273)/gb = AJ006469/ | 1598 | P | 1004.2 | P | 1206.4 | P |
| | | gi = 3687319/us = Mm.20904/len = 1685/STRA = for | | | | | | |
| 11 | 103837_at | Cluster Incl AF092997: Mus musculus caspase-14 mRNA, complete cds/cds = (60,833)/gb = AF092997/gi = 3719426/ | 2715.5 | P | 1493 | P | 1333.3 | P |
| | | ug = Mm 20940/len = 2175/STRA = for | | | | | | |
| 11 | 103858_at | Cluster Incl AW124023: UI-M-BH2.1-apn-b-12-0-UI.s1 Mus musculus cDNA, 3 end/clone = UI-M-BH2.1-apn-b-12-0-UI/ | 178.7 | P | 149.2 | P | 132.9 | P |
| | | clone_end = 3/gb = AW124023/gi = 6099553/ug = Mm.21096/len = 436/STRA = for | | | | | | |
| 11 | 104118_at | Cluster Incl AW123781 UI-M-BH2 1-apm-c-11-0-UI s1 Mus musculus cDNA, 3 end/clone = UI-M-BH2.1-apm-c-11-0-UI/ | 473.6 | P | 247.5 | P | 288.4 | P |
| | | clone_end = 3/gb = AW123781/gi = 6099311/ug = Mm.21972/len = 396/STRA = for | | | | | | |
| 11 | 104143_at | Cluster Incl AI843212 UI-M-AK1-aet-f-07-0-UI s1 Mus musculus cDNA, 3 end/clone = UI-M-AK1-aet-f-07-0-UI/ | 758.7 | P | 573.6 | P | 464 | P |
| | | clone_end = 3/gb = AI843212/gi = 5477425/ug = Mm 22144/len = 368/STRA = for | | | | | | |
| 11 | 104150_at | Cluster Incl AW121957 UI-M-BH2 3-aoi-h-06-0-UI s1 Mus musculus cDNA, 3 end/clone = UI-M-BH2 3-aoi-h-06-0-UI/ | 511.8 | P | 417 | P | 398.8 | P |
| | | clone_end = 3/gb = AW121957/gi = 6097308/ug = Mm 22189/len = 274/STRA = for | | | | | | |
| 11 | 104376_at | Cluster Incl AF006602 Mus musculus histone deacetylase mHDA1 mRNA, complete cds/cds = (0,2975)/gb = AF006602/ | 991.5 | P | 665 | P | 669.6 | P |
| | | gi = 4165859/ug = Mm 22665/len = 3081/STRA = for | | | | | | |
| 11 | 104390_at | Cluster Incl AI836916: UI-M-AJ0-aav-c-12-0-UI.s1 Mus musculus cDNA, 3 end/clone = UI-M-AJ0-aav-c-12-0-UI/ | 626.7 | P | 449.6 | P | 517.9 | P |
| | | clone/end = 3/gb = AI836916/gi = 5471129/ug = Mm 22717/len = 489/STRA = for | | | | | | |
| 11 | 104623_at | Cluster Incl X73360 M. musculus mESG mRNA/cds = (258,2573)/gb = X73360/gi = 313235/ug = Mm 24255/len = 2840/ | 997.9 | P | 388.4 | P | 474.2 | P |
| | | STRA = for | | | | | | |
| 11 | 104624_at | Cluster Incl AI851539 UI-M-BH0-aki-c-09-0-UI.s1 Mus musculus cDNA, 3 end/clone = UI-M-BH0-aki-c-09-0-UI/ | 1063.8 | P | 910.7 | P | 746.1 | P |
| | | clone_end = 3/gb = AI851539/gi = 5495445/ug = Mm.24260/len = 530/STRA = for | | | | | | |
| 11 | 104715_at | Cluster Incl AI152779 ud91a09 r1 Mus musculus cDNA, 5 end/clone = IMAGE-1478200/clone_end = 5/gb = AI152779/ | 1314.6 | P | 1170.8 | P | 1018 | P |
| | | gi = 3681248/ug = Mm.24496/len = 504/STRA = for | | | | | | |
| 11 | 94330_at | Cluster Incl AA710564 vI43b11 r1 Mus musculus cDNA, 5 end/clone = IMAGE-1165821/clone_end = 5/gb = AA710564/ | 1072.4 | P | 419.7 | P | 335.9 | P |
| | | gi = 2720482/ug = Mm.24887/len = 519/STRA = for | | | | | | |
| 11 | 94384_at | Cluster Incl AI847926 UI-M-AI1-afl-c-08-0-UI.s1 Mus musculus cDNA, 3 end/clone = UI-M-AI1-afl-c-08-0-UI/clone_end = 3/ | 1340.7 | P | 1023.4 | P | 1098.5 | P |
| | | gb = AI847926/gi = 5491827/ug = Mm 25279/len = 446/STRA = for | | | | | | |
| 11 | 94410_f_at | Cluster Incl AA611413: vo51e11 r1 Mus musculus cDNA, 5 end/clone = IMAGE-1053452/clone_end = 5/gb = AA611413/ | 929.7 | P | 805.9 | P | 690.5 | P |
| | | gi = 2461492/ug = Mm 25941/len = 402/STRA = rev | | | | | | |
| 11 | 94932_at | Cluster Incl M29464 Platelet derrved growth factor, alpha/cds = (62,652)/gb = M29464/gi = 200272/ug = Mm 2675/len = 906/ | 2251.8 | P | 1586.1 | P | 1598.5 | P |
| | | STRA = for | | | | | | |
| 11 | 95629_at | Cluster Incl AI843679: UI-M-AK1-aer-c-01-0-UI s1 Mus musculus cDNA, 3 end/clone = UI-M-AK1-aer-c-01-0-UI/ | 593.6 | P | 432 | P | 418.6 | P |
| | | clone_end = 3/gb = AI843679/gi = 5477892/ug = Mm 28073/len = 184/STRA = for | | | | | | |
| 11 | 95636_at | Cluster Incl AW123628 UI-M-BH2 1-api-e-09-0-UI s1 Mus musculus cDNA, 3 end/clone = UI-M-BH2 1-api-e-09-0-UI/ | 1795 | P | 698.7 | P | 932.6 | P |
| | | clone_end = 3/gb = AW123628/gi = 6099158/ug = Mm 28094/len = 416/STRA = for | | | | | | |
| 11 | 96195_at | Cluster Incl U57686 Mus musculus p130Cas-related protein Sin mRNA, complete cds/cds = (57,1739)/gb = U57686/ | 644.8 | P | 444.4 | P | 422.8 | P |
| | | gi = 1373343/ug = Mm 28543/len = 2211/STRA = for | | | | | | |
| 11 | 96236_at | Cluster Incl AW122965 UI-M-BH2 1-aoz-g-11-0-UI s2 Mus musculus cDNA, 3 end/clone = UI-M-BH2 1-aoz-g-11-0-UI/ | 614.9 | P | 399.5 | P | 415.5 | P |
| | | clone_end = 3/gb = AW122965/gi = 6098495/ug = Mm 28798/len = 620/STRA = for | | | | | | |
| 11 | 96791 _at | Cluster Incl AW047875 UI-M-BH1-als-g-03-0-UI s1 Mus musculus cDNA, 3 end/clone = UI-M-BH1-als-g-03-0-UI/ | 913.7 | P | 750.7 | P | 795.6 | P |
| | | clone end = 3/gb = AW047875/gi = 5908404/ug = Mm 29116/len = 324/STRA = for | | | | | | |
| 11 | 97330_at | Cluster Incl AI840499 UI-M-AN0-aci-g-08-0-UI.s1 Mus musculus cDNA, 3 end/clone = UI-M-AN0-aci-g-08-0-UI/ | 1892.6 | P | 1707 | P | 1533.1 | P |
| | | clone_end = 3/gb = AI840499/gi = 5474712/ug = Mm 29543/len = 426/STRA = for | | | | | | |
| 11 | 97386_at | Cluster Incl AI853294 UI-M-BH0-aji-f-03-0-UI s1 Mus musculus cDNA, 3 end/clone = UI-M-BH0-aji-f-03-0-UI/clone_end = 3/ | 893.9 | P | 594.9 | P | 796.7 | P |
| | | gb = AI853294/gi = 5497200/ug = Mm 29789/len = 413/STRA = for | | | | | | |
| 11 | 97407_at | Cluster Incl AI848025 UI-M-AH1-agp-e-07-0-UI s1 Mus musculus cDNA, 3 end/clone = UI-M-AH1-agp-e-07-0-UI/ | 381.6 | P | 193.5 | P | 218.2 | P |
| | | clone_end = 3/gb = AI848025/gi = 5491931/ug = Mm 29926/len = 435/STRA = for | | | | | | |
| 11 | 97415_at | Cluster Incl M89777 Mus musculus GTP-binding protein (Rab3D) mRNA, complete cds/cds = (43,702)/gb = M89777/ | 787.5 | P | 397.3 | P | 460.3 | P |
| | | gi = 200631/ug = Mm.29968/len = 703/STRA = for | | | | | | |
| 11 | 97925_at | Cluster Incl AB026241.Mus musculus mRNA for casein kinase, complete cds/cds = (88,1338)/gb = AB028241/gi = 6526519/ | 427.1 | P | 235.5 | P | 338.1 | P |
| | | ug = Mm 30199/len = 1392/STRA = for | | | | | | |
| 11 | 97953 g_at | Cluster Incl U39818 Tuberous sclerosis 2/cds = (90,5534)/gb = U39818/gi = 1236401/ug = Mm 30435/1en = 5637/STRA = for | 155.4 | P | 32.1 | P | 74 | P |
| 11 | 98440_at | Cluster Incl AA596710 vm60d08 r1 Mus musculus cDNA, 5 end/clone = IMAGE-1002639/clone_end = 5/gb = AA596710/ | 1034.8 | P | 387.4 | P | 336.5 | P |
| | | gi = 2412145/ug = Mm 34497/len = 396/STRA = for | | | | | | |
| 11 | 98447_at | Clusler Incl M62362.CCAAT/enhancer binding protein (C/EBP), alpha/cds = (0,1187)/gb = M62362/gi = 192381/ | 3573 | P | 3097.1 | P | 3726.2 | P |
| | | ug = Mm 34537/len = 1188/STRA = rev | | | | | | |
| 11 | 98473_at | Cluster Incl AF032466: Mus musculus arginase II mRNA, complete cds/cds = (34,1098)/gb = AF032466/gi = 2642321/ | 243.1 | P | 198.3 | P | 119.7 | P |
| | | ug = Mm 3506/len = 1363/STRA = for | | | | | | |
| 11 | 99926_at | Cluster Incl AB001489: Phosphatidylinositol glycan, class R/cds = (0,2315)/gb = AB001489/gi = 2804245/ug = Mm 4317/ | 609.1 | P | 478.7 | P | 526.1 | P |
| | | len = 3733/STRA = for | | | | | | |
| 11 | 100410_at | Cluster Incl AW122781 UI-M-BH2 1-aoy-d-01-0-UI.s1 Mus musculus cDNA, 3 end/clone = UI-M-BH2 1-aoy-d-01-0-UI/ | 353.1 | P | 264.8 | P | 269.7 | P |
| | | clone_end = 3/gb = AW122781/gi = 6098311/ug = Mm 46641/len = 162/STRA = for | | | | | | |
| 11 | 100890_at | Cluster Incl AI173038 uh16b06.r1 Mus musculus cDNA, 5 end/clone = IMAGE-1745555/clone_end = 5/gb = AI173038/ | 673 | P | 589.4 | P | 578 | P |
| | | gi = 3720618/ug = Mm 5458/len = 431/STRA = for | | | | | | |
| 11 | 100927_at | Cluster Incl U28960 Phospholipid transfer protein/cds = (0,1475)/gb = U28960/gi = 902887/ug = Mm.6105/len = 1590/ | 431.3 | P | 186.1 | P | 180.8 | P |
| | | STRA = for | | | | | | |
| 11 | 100948_at | Cluster Incl AW049351: UI-M-BH1-amr-h-10-0-UI s1 Mus musculus cDNA, 3 end/clone = UI-M-BH1-amr-h-10-0-UI/ | 1168.6 | P | 845.8 | P | 881 | P |
| | | clone end = 3/gb = AW049351/gi = 5909880/ug = Mm 6403/len = 318/STRA = for | | | | | | |
| 11 | 101430_at | Cluster Incl AW124153 UI-M-BH2.1-apr-10-0-UI s1 Mus musculus cDNA, 3 end/clone = UI-M-BH2 1-apr-f-10-0-UI/ | 1105.3 | P | 622.8 | P | 706.4 | P |
| | | clone_end = 3/gb = AW124153/gi = 6099683/ug = Mm 7556/len = 219/STRA = for | | | | | | |
| 11 | 101441_i_at | Cluster Incl AF031127 Mus musculus inositol trisphosphate receptor type 2 (Itpr2) gene, partial cds/cds = (0,110)/ | 795 | P | 503 | P | 615.2 | P |
| | | gb = AF031127/gi = 2911341/ug = Mm 7800/len = 111/STRA = rev | | | | | | |
| 11 | 101462_r_at | Cluster Incl U06944 Praja1/cds = (328,1602)/gb = U06944/gi = 2828789/ug = Mm 8211/len = 1978/STRA = for | 204.9 | P | 204.2 | P | 153.5 | P |
| 11 | 101930_at | Cluster Incl Y07688: M musculus mRNA for NfiX1-protein/cds = (316,1641)/gb = Y07688/gi = 1524166/ug = Mm 9394/ | 2328.8 | P | 1619.8 | P | 1938.1 | P |
| | | len = 1729/STRA = for | | | | | | |
| 11 | 103485_at | Cluster Incl AA795415: vv06f02.r1 Mus musculus cDNA, 5 end/clone = IMAGE-1210875/clone_end = 5/gb = AA795415/ | 190.1 | P | 22.9 | P | 51.2 | P |
| | | gi = 2858370/ug = Mm.22139/len = 298/STRA = for | | | | | | |
| 11 | 103921_i_at | Cluster Incl AI839690 UI-M-AN0-acp-e-04-0-UI.s1 Mus musculus cDNA, 3 end/clone = UI-M-AN0-acp-e-04-0-UI/ | 555.3 | P | 470 | P | 353.8 | P |
| | | clone_end = 3/gb = AI839690/gi = 5473903/ug = Mm 25034/len = 250/STRA = rev | | | | | | |
| 11 | 103924_at | Cluster Incl AW048884 UI-M-BH1-amf-f-03-0-UI.s1 Mus musculus cDNA, 3 end/clone = UI-M-BH1-amf-f-03-0-UI/ | 786.7 | P | 439.9 | P | 376.2 | P |
| | | clone_end = 3/gb = AW048884/gi = 5909413/ug = Mm.25078/len = 251/STRA = rev | | | | | | |
| 11 | 104016_at | Cluster Incl M91236: Gap junction membrane channel protein beta 5/cds = (120,935)/gb = M91236/gi = 192642/ | 740.1 | P | 316.7 | P | 313.9 | P |
| | | ug = Mm 26859/len = 1272/STRA = for | | | | | | |
| 11 | 104025_at | Cluster Incl AW047185 UI-M-BH1-amb-a-12-0-UI s1 Mus musculus cDNA, 3 end/clone = UI-M-BH1-amb-a-12-0-UI/ | 778.7 | P | 432.4 | P | 493.7 | P |
| | | clone_end = 3/gb = AW047185/gi = 5907714/ug = Mm.26995/len = 416/STRA = for | | | | | | |
| 11 | 104207_at | Cluster Incl AI430272 mf46f12 y1 Mus musculus cDNA, 5 end/clone = IMAGE-408143/clone_end = 5/gb = AI430272/ | 287.7 | P | 230.1 | P | 168.2 | P |
| | | gi = 4276108/ug = Mm 27654/len = 426/STRA = for | | | | | | |
| 11 | 104232_at | Cluster Incl X63099 Gap junction membrane channel protein beta 3/cds = (119,931)/gb = X63099/gi = 50598/ug = Mm 28078/ | 719.5 | P | 782.3 | P | 642.6 | P |
| | | len = 1636/STRA = for | | | | | | |
| 11 | 104255_at | Cluster Incl AA795285 vv03h09 r1 Mus musculus cDNA, 5 end/clone = IMAGE-1210625/clone_end = 5/gb = AA795285/ | 246.9 | P | 232.6 | P | 211.4 | P |
| | | gi = 2858240/ug = Mm 28387/len = 297/STRA = rev | | | | | | |
| 11 | 104400_at | Cluster Incl AF076956 Mus musculus prenylaied SNARE protein Ykt6 (Ykt6) mRNA, complete cds/cds = (15,611)/ | 168 | P | 139.3 | P | 111.4 | P |
| | | gb = AF076956/gi = 3328384/ug = Mm 28680/len = 643/STRA = for | | | | | | |
| 11 | 104434_at | Cluster Incl AW048552 UI-M-BH1-alx-d-07-0-UI s1 Mus musculus cDNA, 3 end/clone = UI-M-BH1-alx-d-07-0-UI/ | 710.9 | P | 449.7 | P | 589.6 | P |
| | | clone_end = 3/gb = AW048552/gi = 5909081/ug = Mm 29241/len = 473/STRA = for | | | | | | |
| 11 | 92310_at | Cluster Incl M96163 Mus musculus (clone 2) serum inducible kinase (SNK) mRNA, mRNA sequence/cds = UNKNOWN/ | 1151.1 | P | 980.3 | P | 934.4 | P |
| | | gb = M96163/gi = 200999/ug = Mm 380/len = 2772/STRA = for | | | | | | |
| 11 | 92517_at | Cluster Incl AB026583 Mus musculus mRNA for Scr3, complete cds/cds = (34,1104)/gb = AB026583/gi = 4730922/ | 115.6 | P | 84.4 | P | 15.9 | P |
| | | ug = Mm 42247/len = 1354/STRA = for | | | | | | |
| 11 | 92726_at | Cluster Incl AJ010605 SRY-box containing gene 6/cds = (0,931)/gb = AJ010605/gi = 3702611/ug = Mm 4656/len = 3371/ | 59.2 | P | −84.5 | P | −20.5 | P |
| | | STRA = for | | | | | | |
| 11 | 93437_f _at | Cluster Incl AI850509 UI-M-BG1-aih-h-10-0-UI s1 Mus musculus cDNA, 3 end/clone = UI-M-BG1-aih-h-10-0-UI/ | 150 | P | 78.9 | P | 102.7 | P |
| | | clone_end = 3/gb = AI850509/gi = 5494415/ug = Mm 65832/len = 453/STRA = for | | | | | | |
| 11 | 93467_at | Cluster Incl AI852734. UI-M-BH0-aji-h-02-0-UI s1 Mus musculus cDNA, 3 end/clone = UI-M-BH0-aji-h-02-0-UI/clone_end = 3/ | 386.9 | P | 227.4 | P | 261.8 | P |
| | | gb = AI852734/gi = 5496640/ug = Mm 6926/len = 348/STRA = for | | | | | | |
| 11 | 93697_at | Cluster Incl U63387 Chromobox homolog 4 (Drosophila Pc class)/cds = (132,1787)/gb = U63387/gi = 2781391/ug = Mm 8556/ | 502.2 | P | 373.7 | P | 254.6 | P |
| | | len = 2542/STRA = for | | | | | | |
| 11 | 94789_r_at | Cluster Incl X04663: Mouse mRNA for beta-tubulin (isotype Mbeta 5)/cds = (78,1412)/gb = X04663/gi = 55047/ug = Mm. 1703/ | 2878.7 | P | 1916.3 | P | 2213.4 | P |
| | | len = 1591/STRA = for | | | | | | |
| 11 | 95952_at | Cluster Incl AW124781: UI-M-BH2.1-aqa-h-01-0-UI s1 Mus musculus cDNA, 3 end/clone = UI-M-BH2 1-aqa-h-01-0-UI/ | 374.9 | P | 313.7 | P | 250 | P |
| | | clone_end = 3/gb = AW124781/gi = 6100311/ug = Mm 24921/len = 422/STRA = rev | | | | | | |
| 11 | 99872_s_at | Cluster Incl L39879: Ferritin light chain 1/cds = (205,756)/gb = L39879/gi = 666913/ug = Mm 7500/len = 920/STRA = for | 4564 | P | 3078.9 | P | 3666.2 | P |
| 11 | 100331_g_at | Cluster Incl AF032714 Mus musculus type II peroxiredoxin protein 2 (PrxII-2) pseudogene, complete sequence/ | 4478 | P | 2551.1 | P | 3343 | P |
| | | cds = (0,596)/gb = AF032714/gi = 3603231/ug = Mm 87831/len = 597/STRA = for | | | | | | |
| 11 | 100342_l_at | Cluster Incl M28729: Tubulin alpha 1/cds = (0,596)/gb = M28729/gi = 202222/ug = Mm 88110/len = 786/STRA = for | 5977.3 | P | 3786.6 | P | 3551.7 | P |
| 11 | 95232_at | Cluster Incl AB009392 Mus musculus mRNA for protein L, partial cds/cds = (0,600)/gb = AB009392/gi = 2723394/ | 1859.1 | P | 1347.5 | P | 1932.5 | P |
| | | ug = Mm 9043/len = 958/STRA = for | | | | | | |
| 11 | 95795_at | Cluster Incl U96810 Mus musculus Supt4h2 gene, complete cds/cds = (0,353)/gb = U96810/gi = 3779195/ug = Mm 87825/ | 697.9 | P | 465.3 | P | 593.9 | P |
| | | len = 354/STRA = for | | | | | | |
| 11 | 96426_at | Cluster Incl U38967 Prothymosin beta 4/cds = (0,134)/gb = U38967/gi = 1072099/ug = Mm 22566/len = 542/STRA = for | 2792.3 | P | 1785.8 | P | 2197.9 | P |
| 11 | 101123_at | Cluster Incl U76253: Integral membrane protein 2 B/cds = (145,945)/gb = U76253/gi = 3746126/ug = Mm 4266/len = 1790/ | 3236.3 | P | 1756.3 | P | 1891 | P |
| | | STRA = for | | | | | | |
| 11 | 101607_at | Cluster Incl C80068 C80068 Mus musculus cDNA, 3 end/clone = J0076B01/clone_end = 3/gb = C80068/gi = 2520398/ | 322.5 | P | 204.7 | P | 199 | P |
| | | ug = Mm 5905/len = 287/STRA = rev | | | | | | |
| 11 | 102134_f_at | Cluster Incl AI461702 vw57d12.x1 Mus musculus cDNA, 3 end/clone = Image-1247927/clone_end = 3/gb = AI461702/ | 840.9 | P | 550.8 | P | 594.2 | P |
| | | gi = 4315732/ug = Mm.43556/len = 470/STRA = rev | | | | | | |
| 11 | 92610_at | Cluster Incl M21332 Mouse MHC class III RD gene (H2-d and H2-Sk haplotypes), complete cds/cds = (0,1127)/gb = M21332/ | 375.8 | P | 59.9 | A | 159.4 | P |
| | | gi = 199607/ug = Mm.10964/len = 1128/STRA = for | | | | | | |
| 11 | 93300_at | Cluster Incl X57413 Transforming growth factor, beta 2/cds = (1217,2461)/gb = X57413/gi = 54772/ug = Mm 18213/len = 4267/ | 104.7 | P | −3.2 | P | 16.1 | P |
| | | STRA = rev | | | | | | |
| 11 | 93598_at | Cluster Incl AI840437 UI-M-AN0-aci-a-04-0-UI s1 Mus musculus cDNA, 3 end/clone = UI-M-AN0-aci-04-0-UI/ | 110.9 | P | 94.4 | P | 94.5 | P |
| | | clone_end = 3/gb = AI840437/gi = 5474650/ug = Mm.20847/len = 287/STRA = for | | | | | | |
| 11 | 93967_at | Cluster Incl AI850979 UI-M-BH0-akd-h-04-0-UI.s1 Mus musculus cDNA, 3 end/clone = UI-M-BH0-akd-h-04-0-UI/ | 756.6 | P | 604.1 | P | 558.7 | P |
| | | clone_end = 3/gb = AI850979/gi = 5494885/ug = Mm.21645/len = 216/STRA = for | | | | | | |
| 11 | 94277_at | Cluster Incl U66257. Mus musculus thrombospodin 3 and metaxin genes, exon 1, partial cds, and intergenic region/ | 301.9 | P | 206.9 | M | 212.2 | P |
| | | cds = (0,81)/gb = U66257/gi = 1575552/ug = Mm 22508/len = 82/STRA = for | | | | | | |
| 11 | 94807_at | Cluster Incl AI848354 UI-M-AH1-ago-h-07-0-UI s1 Mus musculus cDNA, 3 end/clone = UI-M-AH1-ago-h-07-0-UI/ | 670.7 | P | 708.9 | P | 737 | P |
| | | clone_end = 3/gb AI848354/gi = 5492300/ug = Mm 22679/len = 221/STRA = for | | | | | | |
| 11 | 95512_at | Cluster Incl M60320 Protein-L-isoaspartate (D-aspartate) O-Methyltransferase 1/cds = (119,802)/gb = M60320/gi = 200254/ | 215.3 | P | 41.2 | P | 79.4 | P |
| | | ug = Mm 25293/len = 1580/STRA = for | | | | | | |
| 11 | 97807_at | Cluster Incl AI838630 UI-M-AO0-aca-a-04-0-UI s1 Mus musculus cDNA, 3 end/clone = UI-M-AO0-aca-a-04-0-UI/ | 157 | P | 82.9 | P | 123.6 | P |
| | | clone_end = 3/gb = AI838630/gi = 5472843/ug = Mm.2815/len = 430/STRA = for | | | | | | |
| 11 | 98938_at | Cluster Incl AI852184 UI-M-BH0-ajc-a-05-0-UI s1 Mus musculus cDNA, 3 end/clone = UI-M-BHo-ajc-a-05-0-UI/ | 367.3 | P | 224.4 | P | 252.4 | P |
| | | clone_end = 3/gb = AI852184/gi = 5496090/ug = Mm 28690/len = 258/STRA = for | | | | | | |
| 11 | 96873_at | Cluster Incl AI843287: UI-M-AO1-aei-e-06-0-UI s1 Mus musculus cDNA, 3 end/clone = UI-M-AO1-aei-e-06-0-UI/ | 569.1 | P | 416.2 | P | 549.7 | P |
| | | clone_end = 3/gb = AI843287/gi = 5477500/ug = Mm 30069/len = 425/STRA = for | | | | | | |
| 11 | 96939_at | Cluster Incl AI842649: UI-M-AQ1-aed-g-07-0-UI.s1 Mus musculus cDNA, 3 end/clone = UI-M-AQ1-aed-g-07-0-UI/ | 491.2 | P | 189.4 | M | 220.9 | P |
| | | clone_end = 3/gb = AI842649/gi = 5476862/ug = Mm.30188/len = 312/STRA = for | | | | | | |
| 11 | 102327_at | Cluster Incl AF078705: Amine oxidase, copper containing 3/cds = (0,2297)/gb = AF078705/gi = 3603354/ug = Mm 10734/ | 400.9 | P | 224.1 | P | 265.7 | P |
| | | len = 2298/STRA = for | | | | | | |
| 11 | 102783_at | Cluster Incl AI131744 ue24h11.x1 Mus musculus cDNA, 3 end/clone = IMAGE-1481349/clone_end = 3/gb-AI131744/ | 966.5 | P | 763.8 | P | 589.6 | P |
| | | gi = 3601760/ug = Mm 13773/len = 472/STRA = for | | | | | | |
| 11 | 103001_at | Cluster Incl U43836 Mus musculus VEGF-related factor mvrf186 precursor mRNA, complete cds/cds = (192,815)/ | 362 | P | 270 | P | 338.3 | P |
| | | gb = U43836/gi = 1703480/ug = Mm 15607/len = 1236/STRA = for | | | | | | |
| 11 | 104742_at | Cluster Incl AA919832: vy18b09 r1 Mus musculus cDNA, 5 end/clone = IMAGE-1295801/clone_end = 5/gb = AA919832/ | 449.2 | P | 229.1 | M | 238.5 | P |
| | | gi = 3066611/ug = Mm 24679/len = 474/STRA = for | | | | | | |
| 11 | 97928_at | Cluster Incl AW045278 UI-M-BH1-akn-c-07-0-UI s1 Mus musculus cDNA, 3 end/clone = UI-M-BH1-akn-c-07-0-UI/ | 354.8 | P | 142.7 | P | 158.2 | P |
| | | clone_end = 3/gb = AW045278/gi = 5905807/ug = Mm 30232/len = 250/STRA = for | | | | | | |
| 11 | 98004_at | Cluster Incl M63554: Mus musculus inhibitor protein of cAMP-dependent protein kinase, complete cds/cds = (29,259)/ | 85.8 | P | 40.8 | P | 51.7 | P |
| | | gb = M63554/gi = 200384/ug = Mm.3193/len = 2278/STRA = for | | | | | | |
| 11 | 103755_at | Cluster Incl D89677 Mus musculus mRNA for Kryn, complete cds/cds = (1123,3087)/gb = D89677/gi = 1944454/ | 130.8 | P | 230.3 | P | 166.4 | P |
| | | ug = Mm 2454/len = 4936/STRA = for | | | | | | |
| 11 | 104169_at | Cluster Incl D32167 Zinc finger protein of the cerebellum 1/cds = (530,1873)/gb = D32167/gi = 565654/ug = Mm 2719/ | 481.4 | P | 84.3 | P | 169.4 | P |
| | | len = 2947/STRA = for | | | | | | |
| 11 | 92180_at | Cluster Incl AI851599 UI-M-BH0-ain-a-02-0-UI s1 Mus musculus cDNA, 3 end/clone = UI-M-BH0-ain-a-02-0-UI/ | 404.8 | P | 89.8 | P | 88.6 | A |
| | | clone_end = 3/gb = AI851599/gi = 5495505/ug = Mm 33796/len = 352/STRA = for | | | | | | |
| 11 | 92322_at | Cluster Incl X94353 Cathelin-like protein/cds = (14,535)/gb = X94353/gi = 1177533/ug = Mm 3834/len = 593/STRA = for | 817.5 | P | 558.2 | P | 604.2 | P |
| 11 | 94801_at | Cluster Incl AI607332 ub76a01 x1 Mus musculus cDNA, 3 end/clone = IMAGE-1383624/clone_end = 3/gb = AI607332/ | 143 | P | 85.8 | P | 43.9 | P |
| | | gi = 4616499/ug = Mm 1823/len = 431/STRA = for | | | | | | |
| 11 | AFFX- | X57349 M musculus mRNA for transferrin receptor (_5, _M, _3 represent transcript regions 5 prime, | 262.9 | P | 103.7 | P | 132.7 | P |
| | TransRecMur/ | | | | | | | |
| | X57349_3_at | Middle, and 3 prime respectively) | | | | | | |
| 11 | 93009_at | Cluster Incl J04696 Mouse glutathione S-transferase class mu (GST5-5) mRNA, complete cds/cds = (4,660)/gb = J04696/ | 532.6 | P | 220.9 | A | 275.5 | P |
| | | gi = 193549/ug = Mm 14601/len = 1014/STRA = for | | | | | | |
| 11 | 94217_f_at | Cluster Incl AC002397 Mouse chromosome 6 BAC-284H12 (Research Genetics mouse BAC library) complete sequence/ | 294.6 | P | 255.8 | A | 236.6 | P |
| | | cds = (198,998)/gb = AC002397/gi = 3287367/ug = Mm 22228/len = 1155/STRA = for | | | | | | |
| 11 | 95501_at | Cluster Incl AW122970 UI-M-BH2 1-aoz-h-06-0-UI s2 Mus musculus cDNA, 3 end/clone = UI-M-BH2.1-aoz-h-06-0-UI/ | 254.3 | P | 204.9 | P | 120.9 | M |
| | | clone_end = 3/gb = AW122970/gi = 6098500/ug = Mm 25157/len = 435/STRA = for | | | | | | |
| 11 | 99085_at | Cluster Incl AI021421: ub12a08 r1 Mus musculus cDNA, 5 end/clone = IMAGE-1366742/clone_end = 5/gb = AI021421/ | 272.9 | A | 160.1 | P | 262 | P |
| | | gi = 3235757/ug = Mm 38976/len = 470/STRA = for | | | | | | |
| 11 | 99111_at | Cluster Incl U09874 Mus musculus SKD3 mRNA, complete cds/cds = (45,2078)/gb = U09874/gi = 563128/ug = Mm 3990/ | 248.7 | P | 195.7 | A | 201.6 | P |
| | | len = 2138/STRA = rev | | | | | | |
| 11 | 100109_s_at | Cluster Incl X03492 Keratin complex 1, acidic, gene 13/cds = (0,938)/gb = X03492/gi = 52782/ug = Mm 4646/len = 1204/ | 256.6 | P | 189.2 | P | 146.7 | P |
| | | STRA = for | | | | | | |
| 11 | 102022_at | Cluster Incl AW124555 UI-M-BH2 1-apt-b-05-0-UI s1 Mus musculus cDNA, 3 end/clone = UI-M-BH2 1-apt-b-05-0-UI/ | 179.2 | P | 137.6 | M | 122.1 | M |
| | | clone_end = 3/gb = AW124555/gi = 6100085/ug = Mm.10093/len = 547/STRA = for | | | | | | |
| 11 | 104135_at | Cluster Incl AW045474 UI-M-BH1-akr-c-02-0-UI s1 Mus musculus cDNA, 3 end/clone = UI-M-BH1-akr-c-02-0-UI/ | 96.3 | P | −45.8 | A | −57.4 | A |
| | | clone_end = 3/gb = AW045474/gi = 5905968/ug = Mm 22085/len = 318/STRA = for | | | | | | |
| 11 | 94983_at | Cluster Incl AI851614 UI-M-BH0-ain-b-07-0-UI s1 Mus musculus cDNA, 3 end/clone = UI-M-BH0-ain-b-07-0-UI/ | 206.8 | P | 188.9 | A | 218.2 | P |
| | | clone_end = 3/gb = AI851614/gi = 5495520/ug = Mm 27282/len = 425/STRA = for | | | | | | |
| 11 | 97995_at | Cluster Incl X61385 Transcription factor 7, T-cell specific/cds = (189,1100)/gb = X61385/gi = 54760/ug = Mm.31630/len = 1790/ | 148.8 | P | 90.1 | M | 80.9 | P |
| | | STRA = for | | | | | | |
| 11 | 102952_g_at | Cluster Incl AJ224738 Mus musculus mRNA for death adaptor molecule (Raidd/Cradd), clone 6-1/cds = (118,717)/ | 150.2 | P | 92.7 | A | 89.3 | P |
| | | gb = AJ224738/gi = 3929835/ug = Mm 17493/len = 1649/STRA = for | | | | | | |
| 11 | 93431_at | Cluster Incl Z38015 Dystrophia myotonica kinase, B15/cds = (143,2038)/gb = Z38015/gi = 563525/ug = Mm 6529/len = 2755/ | 476.4 | A | 240.4 | P | 352.3 | P |
| | | STRA = for | | | | | | |
| 11 | 93587_at | Cluster Incl L13129 Annexin VII/cds = (42,1433)/gb = L13129/gi = 293293/ug = Mm.20794/len = 2636/STRA = for | 141.7 | M | 105.2 | P | 143 | P |
| 11 | 95502_at | Cluster Incl AI840267: UI-M-AH0-act-e-08-0-UI s1 Mus musculus cDNA, 3 end/clone = UI-M-AH0-act-e-08-0-UI/ | 210.3 | A | 214.2 | P | 238.3 | A |
| | | clone_end = 3/gb = AI840267/gi = 5474480/ug = Mm.25161/len = 358/STRA = for | | | | | | |
| 11 | 97886_at | Cluster Incl AI530375 ui92c09 y1 Mus musculus cDNA, 5 end/clone = IMAGE-1889872/clone_end = 5/gb = AI530375/ | 209.2 | A | 90.5 | P | 146.9 | M |
| | | gi = 4444510/ug = Mm 28393/len = 474/STRA = for | | | | | | |
| 11 | 102848_f_at | Cluster Incl AI840577 UI-M-AM0-adp-h-10-0-U1.s1 Mus musculus cDNA, 3 end/clone = UI-M-AM0-adp-h-10-0-UI/ | 295.1 | P | 25.3 | A | 66.2 | P |
| | | clone_end = 3/gb = AI840577/gi = 5474790/ug = Mm 14808/len = 432/STRA = for | | | | | | |
| 11 | 94403_at | Cluster Incl AW121330 UI-M-BH2 2-aom-b-01-0-UI s1 Mus musculus cDNA, 3 end/clone = UI-M-BH2 2-aom-b-01-0-UI/ | 285.9 | A | 107.9 | A | 158.2 | P |
| | | clone_end = 3/gb = AW121330/gi = 6096663/ug = Mm 25799/len = 555/STRA = for | | | | | | |
| 11 | 96240_at | Cluster Incl AI851309 UI-M-BH0-akf-h-03-0-UI s1 Mus musculus cDNA, 3 end/clone = UI-M-BH0-akf-h-03-0-UI/ | 85 | P | 23.9 | A | 3.7 | A |
| | | clone_end = 3/gb = AI851309/gi = 5495215/ug = Mm 28819/len = 207/STRA = for | | | | | | |
| 11 | 99490_at | Cluster Incl AW050348 UI-M-BH1-ang-d-06-0-UI s1 Mus musculus cDNA, 3 end/clone = UI-M-BH1-ang-d-06-0-UI/ | 142.8 | P | 77.4 | A | 66.3 | A |
| | | clone_end = 3/gb = AW050348/gi = 5910877/ug = Mm 41531/len = 493/STRA = for | | | | | | |
| 11 | 102824_at | Cluster Incl AF031035: Mus musculus stanniocalcin (Stc2) mRNA, complete cds/cds = (199,1089)/gb = AF031035/ | 156.7 | M | 8 | A | 165.9 | P |
| | | gi = 4104012/ug = Mm 12904/len = 1793/STRA = for | | | | | | |
| 11 | 101350_g_at | Cluster Incl U73170: Polo-like kinase, pseudogene 1/cds = (0,1788)/gb = U73170/gi = 2580507/ug = Mm 57017/len = 1789/ | 394.5 | P | 385.2 | P | 306.5 | P |
| | | STRA = for | | | | | | |
| 11 | 101182_at | Cluster Incl X95255 GLI-Kruppel family member GLI3/cds = (171,4981)/gb = X95255/gi = 1419013/ug = Mm 5098/len = 5113/ | 76.5 | P | 49.5 | P | 24.9 | A |
| | | STRA = for | | | | | | |
| 11 | 94518_at | Cluster Incl AW121749 UI-M-BH2 3-aoe-c-12-0-UI s1 Mus musculus cDNA, 3 end/clone = UI-M-BH2 3-aoe-c-12-0-UI/ | 416.2 | A | 221.2 | A | 365.9 | P |
| | | clone_end = 3/gb = AW121749/gi = 6097084/ug = Mm.28999/len = 313/STRA = for | | | | | | |
| 11 | 98590_at | Cluster Incl D89571 Mus musculus mRNA for ryudocan core protein, complete cds/cds = (24,620)/gb = D89571/gi = 2373476/ | 333.4 | A | 40.9 | A | 275.8 | P |
| | | ug = Mm 3815/len = 2456/STRA = for | | | | | | |
| 11 | 100539_at | Cluster Incl AI841279: UI-M-AM0-adu-e-08-0-UI.s1 Mus musculus cDNA, 3 end/clone = UI-M-AM0-adu-e-08-0-UI/ | 208.8 | A | 150.2 | P | 157 | A |
| | | clone_end = 3/gb = AI841279/gi = 5475492/ug = Mm.5293/len = 439/STRA = for | | | | | | |
| 11 | 103671_at | Cluster Incl AF061972 Mus musculus Tat-Interacting protein TIP30 mRNA, complete cds/cds = (191,919)/gb = AF061972/ | 104.6 | P | 17.6 | M | 21.3 | P |
| | | gi = 3820524/ug = Mm 20801/len = 1178/STRA = for | | | | | | |
| 11 | 95536_at | Cluster Incl AB025015: Mus musculus mRNA for elongin A, complete cds/cds = (8,2329)/gb = AB025015/gi = 6009623/ | 301 | A | 164.5 | P | 294 | P |
| | | ug = Mm 27663/len = 2537/STRA = for | | | | | | |
| 11 | 99933_at | Cluster Incl D26107: Mouse mRNA for poliovirus receptor homolog protein soluble form, complete cds/cds = (0,1592)/ | 189.2 | P | 147.2 | A | 20.1 | A |
| | | gb = D26107/gi = 475017/ug = Mm.4341/len = 2443/STRA = for | | | | | | |
| 11 | 102268_at | Cluster Incl AW123015 UI-M-BH2 1-apb-e-05-0-UI s1 Mus musculus cDNA, 3 end/clone = UI-M-BH2.1-apb-e-05-0-UI/ | 447.6 | P | 410.3 | P | 331 | M |
| | | clone_end = 3/gb = AW123015/gi = 6098545/ug = Mm 1116/len = 449/STRA = for | | | | | | |
| 11 | 102643_at | Cluster Incl M93148 Homeo box A2/cds = (308,1426)/gb = M93148/gi = 193910/ug = Mm 131/len = 1460/STRA = for | 171 | P | 135.8 | A | 134.9 | A |
| 11 | 102943_at | Cluster Incl AI099344 ue37d12 y1 Mus musculus cDNA, 5 end/clone = IMAGE-1482551/clone_end = 5/gb = AI099344/ | 195.7 | P | 54.7 | A | 49.1 | P |
| | | gi = 3448869/ug = Mm 17231/len = 364/STRA = for | | | | | | |
| 11 | 103933_at | Cluster Incl U61183: Yolk sac gene 2/cds = (12,1637)/gb = U61183/gi = 1628564/ug = Mm 2517/len = 2106/STRA = for | −18.7 | P | −94.4 | A | −27.7 | A |
| 11 | 102137_f_at | Cluster Incl AI845856 UI-M-AO1-aeg-e-12-0-UI s1 Mus musculus cDNA, 3 end/clone = UI-M-AO1-aeg-e-12-0-UI/ | 894 | P | 734.9 | A | 705.6 | A |
| | | clone_end = 3/gb = AI845856/gi = 5489762/ug = Mm 43942/len = 309/STRA = rev | | | | | | |
| 11 | 95061_at | Cluster Incl AI838996 UI-M-AN0-acm-b-03-0-UI s1 Mus musculus cDNA, 3 end/clone = UI-M-AN0-acm-b-03-0-UI/ | 52.9 | P | 38.8 | A | 0.7 | A |
| | | clone_end = 3/gb = AI638996/gi = 5473209/ug = Mm.29162/len = 417/STRA = for | | | | | | |
| 11 | 94372_at | Cluster Incl D49956 Mouse mRNA lor 8-oxo-dGTPase, complete cds/cds = (98,568)/gb = D49956/gi = 1065597/ | 357.7 | P | 107 | A | 246.6 | P |
| | | ug = Mm.2539/len = 939/STRA = for | | | | | | |
| 11 | 96833_at | Cluster Incl AW048468 UI-M-BH1-alj-b-03-0-UI s1 Mus musculus cDNA, 3 end/clone = UI-M-BH1-alj-b-03-0-UI/ | 341.8 | A | 275.4 | P | 188 | A |
| | | clone_end = 3/gb = AW048468/gi = 5908997/ug = Mm.29420/len = 333/STRA = for | | | | | | |
| 11 | 101382_at | Cluster Incl AF020198 Mus musculus transcription factor PBX2 (PBX2) mRNA, complete cds/cds = (85,1377)/ | 459.9 | P | 159.9 | A | 173.6 | A |
| | | gb = AF020198/gi = 2432012/ug = Mm 7103/len = 2805/STRA = for | | | | | | |
| 11 | 101891_at | Cluster Incl Y09517. M musculus mRNA for 17-beta-hydroxysteroid dehydrogenase type II/cds = (94,1239)/gb = Y09517/ | 193.2 | P | 140.2 | A | 142.2 | A |
| | | gi = 2198500/ug = Mm 8877/len = 1342/STRA = for | | | | | | |
| 11 | 92441_at | Cluster Incl Y10007 Fibroblast activation protein/cds = (170,2455)/gb = Y10007/gi = 1743329/ug = Mm 41816/len = 2715/ | 113.7 | P | 30.9 | A | 39.6 | A |
| | | STRA = for | | | | | | |
| 11 | 93952_r_at | Cluster Incl AA409629 EST01370 Mus musculus cDNA, 3 end/clone = c0009A09/clone_end = 3/gb = AA409629/ | 36.2 | P | −11.8 | A | −5.5 | A |
| | | gi = 2066285/ug = Mm.94218/len = 305/STRA = rev | | | | | | |
| 11 | 102094_f_at | Cluster Incl AI841270 UI-M-AM0-adu-d-10-0-UI s1 Mus musculus cDNA, 3 end/clone = UI-M-AM0-adu-d-10-0-UI/ | 543.2 | A | 402.1 | P | 313.2 | A |
| | | clone_end = 3/gb = AI841270/gi = 5475483/ug = Mm 42353/len = 210/STRA = rev | | | | | | |
| 11 | 101980_at | Cluster Incl J03750 Mouse single stranded DNA binding protein p9 mRNA, complete cds/cds = UNKNOWN/gb = J03750/ | 150.8 | A | 132.7 | A | 60.2 | M |
| | | gi = 192235/ug = Mm.966/len = 2777/STRA = for | | | | | | |
| 11 | 103607_at | Cluster Incl AI882309 ub98g02 r1 Mus musculus cDNA, 5 end/clone = IMAGE-1396562/clone_end = 5/gb = AI882309/ | 258 | P | 124 | A | 116.9 | P |
| | | gi = 5567398/ug = Mm.20164/len = 487/STRA = for | | | | | | |
| 11 | 94406_at | Cluster Incl AJ242864: Mus musculus mRNA 2 for phtf protein/cds = (561,2846)/gb = AJ242864/gi = 6523390/ug = Mm 25877/ | 264.5 | P | 161.3 | A | 126.7 | A |
| | | len = 3330/STRA = for | | | | | | |
| 11 | 96206_at | Cluster Incl AI854506 UI-M-BH0-ajj-b-09-0-UI s1 Mus musculus cDNA, 3 end/clone = UI-M-BH0-ajj-b-09-0-UI/clone_end = 3/ | 581 | A | 300.9 | A | 405.2 | A |
| | | gb = AI854506/gi = 5498412/ug = Mm 28639/len = 413/STRA = for | | | | | | |
| 11 | 97990_at | Cluster Incl D85923 Myosin heavy chain 11, smooth muscle/cds = (104,6022)/gb = D85923/gi = 1945077/ug = Mm 3153/ | 163.6 | A | 231.2 | P | 83.6 | P |
| | | len = 6175/STRA = rev | | | | | | |
| 11 | 103708_at | Cluster Incl AI132207: ue28g02.x1 Mus musculus cDNA, 3 end/clone = IMAGE-1481714/clone_end = 3/gb = AI132207/ | 138.9 | A | −2.5 | A | 3.1 | A |
| | | gi = 3602223/ug = Mm 24090/len = 450/STRA = for | | | | | | |
| 11 | 104395_at | Cluster Incl AW121826 UI-M-BH2.3-anx-e-08-0-UI s2 Mus musculus cDNA, 3 end/clone = UI-M-BH2 3-anx-e-08-0-UI/ | 516.7 | A | 278.1 | A | 273.8 | A |
| | | clone_end = 3/gb = AW121826/gi = 6097196/ug = Mm 28604/len = 413/STRA = for | | | | | | |
| 11 | 104681_at | Cluster Incl U96963 Diaphanous homolog 1 (Drosophila)/cds = (93,3880)/gb = U96963/gi = 2114472/ug = Mm.3282/len = 4378/ | 317.3 | P | 216.4 | M | 189.2 | P |
| | | STRA = for | | | | | | |
| 11 | 94140_at | Cluster Incl M59446 Scavenger receptor/cds = (0,20)/gb = M59446/gi = 192739/ug = Mm 1227/len = 413/STRA = for | 360.9 | A | 216.2 | A | 171.9 | A |
| 11 | 97184_at | Cluster Incl AI528219 uI94h04.y1 Mus musculus cDNA, 5 end/clone = IMAGE-1890103/clone_end = 5/gb = AI528219/ | 208.6 | P | 148.5 | P | 132.1 | A |
| | | gi = 4442354/ug = Mm 36077/len = 505/STRA = for | | | | | | |
| 11 | 101196_at | Cluster Incl D50060. Proprotein convertase subtilisin/kexin type 6/cds = (0,2798)/gb = D50060/gi = 769700/ug = Mm 5182/ | 173 | P | 148.4 | A | 55.3 | A |
| | | len = 3063/STRA = for | | | | | | |
| 11 | 102120_f_at | Cluster Incl C80148: C80148 Mus musculus cDNA, 3 end/clone = J0077C02/clone_end = 3/gb = C80148/gi = 2520478/ | 1141.6 | A | 717.7 | M | 660.2 | A |
| | | ug = Mm 42674/len = 583/STRA = rev | | | | | | |
| 12 | 92544_f_at | Cluster Incl AF055983. Mus musculus proteasome alpha7/C8 subunit mRNA, complete cds/cds = (42,809)/gb = AF055983/ | 1484.3 | P | 2145.7 | P | 2102.9 | P |
| | | gi = 3037090/ug = Mm 1007/len = 938/STRA = for | | | | | | |
| 12 | 92562_at | Cluster Incl U70475: Nuclear, factor, erythroid derived 2, like 2/cds = (139,1932)/gb = U70475/gi = 1778293/ug = Mm 1025/ | 151.3 | P | 382.9 | P | 352.5 | P |
| | | len = 2380/STRA = for | | | | | | |
| 12 | 92564_at | Cluster Incl AI891475 uI59a06 x1 Mus musculus cDNA, 3 end/clone = IMAGE-2123314/clone_end = 3/gb = AI891475/ | 98.6 | P | 196.4 | P | 171.3 | P |
| | | gi = 5597377/ug = Mm 10272/len = 493/STRA = for | | | | | | |
| 12 | 92565_at | Cluster Incl AI847109 UI-M-AP1-agn-g-04-0-UI s1 Mus musculus cDNA, 3 end/clone = UI-M-AP1-agn-g-04-0-UI/ | 256.8 | P | 433.7 | P | 341.3 | P |
| | | clone_end = 3/gb = AI847109/gi = 5491015/ug = Mm 10278/len = 324/STRA = for | | | | | | |
| 12 | 92569_f_at | Cluster Incl AF053232 Mus musculus SIK similar protein mRNA, complete cds/cds = (270,1691)/gb = AF053232/ | 163.5 | P | 497.6 | P | 292.6 | P |
| | | gi = 2996193/ug = Mm 10303/len = 1879/STRA = for | | | | | | |
| 12 | 92596_at | Cluster Incl U97327. Mus musculus calcyclin binding protein (CACYBP) mRNA, partial cds/cds = (0,744)/gb = U97327/ | 67.9 | P | 456.1 | P | 274 | P |
| | | gi = 3138917/ug = Mm. 10702/len = 1404/STRA = for | | | | | | |
| 12 | 92797_at | Cluster Incl AI840051: UI-M-AL0-abl-a-01-0-UI s1 Mus musculus cDNA, 3 end/clone = UI-M-AL0-abI-a-01-0-UI/ | 352.4 | P | 646 | P | 652.3 | P |
| | | clone_end = 3/gb = AI840051/gi = 5474264/ug = Mm 12665/len = 450/STRA = for | | | | | | |
| 12 | 92831_at | Cluster Incl AI846308: UI-M-AK1-aez-g-10-0-UI s1 Mus musculus cDNA, 3 end/clone = UI-M-AK1-aez-g-10-0-UI/ | 324.1 | P | 526 | P | 522.8 | P |
| | | clone_end = 3/gb = AI846308/gi = 5490214/ug = Mm 12983/len = 263/STRA = for | | | | | | |
| 12 | 93045_at | Cluster Incl L28836: Peroxisomal membrane protein, 70 kDa/cds = (0,1979)/gb = L28836/gi = 454834/ug = Mm.1519/ | 375.1 | P | 526.3 | P | 445.9 | P |
| | | len = 1980/STRA = for | | | | | | |
| 12 | 93058_at | Cluster Incl AF026481 Mus musculus elF-1A (elF-1A) mRNA, complete cds/cds = (1172,1606)/gb = AF026481/gi = 3746339/ | 342.6 | P | 478.9 | P | 314.3 | P |
| | | ug = Mm 1540/len = 2663/STRA = for | | | | | | |
| 12 | 93111_at | Cluster Incl D45836 Mouse mRNA for nuclear pore-targeting complex component of 97 kDa, complete cds/cds = (198,2828)/ | 442 | P | 698.7 | P | 722.2 | P |
| | | gb = D45836/gi = 871889/ug = Mm 16710/len = 3897/STRA = for | | | | | | |
| 12 | 93120_f_at | Cluster Incl V00746 Mus musculus H-2K gene for MHC class I antigen H-2K (allele b), exons 1 to 3 and joined CDS/ | 1309.9 | P | 2097.4 | P | 1661 | P |
| | | cds = (0,1109)/gb = V00746/gi = 51154/ug = Mm 16771/len = 1110/STRA = rev | | | | | | |
| 12 | 93277_at | Cluster Incl X53584 Mouse mRNA for HSP60 protein (clones 3T3-7, −9, and −M1)/cds = (0,1667)/gb = X53584/gi = 51451/ | 1432.5 | P | 2626.2 | P | 1933.6 | P |
| | | ug = Mm 1777/len = 2119/STRA = for | | | | | | |
| 12 | 93310_at | Cluster Incl AI836423 UI-M-AI0-aak-b-03-0-UI s1 Mus musculus cDNA, 3 end/clone = UI-M-AI0-aak-b-03-0-UI/ | 1203.5 | P | 2039 | P | 1640.1 | P |
| | | clone_end = 3/gb = AI836423/gi = 5470636/ug = Mm 18472/len = 565/STRA = for | | | | | | |
| 12 | 93547_at | Cluster Incl L03279 Core binding factor beta/cds = UNKNOWN/gb = L03279/gi = 200177/ug = Mm 2018/len = 2685/STRA = for | 1886.3 | P | 2188.5 | P | 2069.3 | P |
| 12 | 93600_at | Cluster Incl AJ011565 Mus musculus mRNA for leptin receptor gene-related protein/cds = (0,395)/gb = AJ011565 | 560 | P | 747.1 | P | 664.9 | P |
| | | gi = 3688429/ug = Mm 20879/len = 635/STRA = for | | | | | | |
| 12 | 93727_at | Cluster Incl AB003502 Mus musculus mRNA for Guanine Nucleotide Regulatory Protein, partial cds/cds = (0,1652)/ | 302.4 | P | 560.1 | P | 455.2 | P |
| | | gb = AB003502/gi = 3461879/ug = Mm 20925/len = 2079/STRA = for | | | | | | |
| 12 | 93729_at | Cluster Incl Y15197: Mus musculus mRNA for E-MAP-115 protein/cds = (0,2192)/gb = Y15197/gi = 3417385/ug = Mm 20928/ | 99.1 | P | 141.3 | P | 108 | P |
| | | len = 2893/STRA = for | | | | | | |
| 12 | 93747_at | Cluster Incl AW122599 UI-M-BH2.2-aox-h-11-0-UI s1 Mus musculus cDNA, 3 end/clone = UI-M-BH2 2-aox-h-11-0-UI/ | 636.9 | P | 882.5 | P | 1006.4 | P |
| | | clone_end = 3/gb = AW122599/gi = 6098125/ug = Mm 21092/len = 456/STRA = for | | | | | | |
| 12 | 93826_at | Cluster Incl AI956230: ul73a07 y1 Mus musculus cDNA, 5 end/clone = IMAGE-2136180/clone_end = 5/gb = AI956230/ | 539.1 | P | 868 | P | 940.4 | P |
| | | gi = 5748967/ug = Mm 21544/len = 513/STRA = for | | | | | | |
| 12 | 93829_at | Cluster Incl AW107884: um18c08 x 1 Mus musculus cDNA, 3 end/clone = IMAGE-2192654/clone_end = 3/gb = AW107884/ | 998.5 | P | 2210.5 | P | 1322.7 | P |
| | | gi = 6078684/ug = Mm 21556/len = 579/STRA = for | | | | | | |
| 12 | 93832_at | Cluster Incl AI854541: UI-M-BH0-ajj-e-12-0-UI s1 Mus musculus cDNA, 3 end/clone = UI-M-BH0-ajj-o-12-0-UI/clone_end = 3/ | 255 | P | 989.6 | P | 499.9 | P |
| | | gb = AI854541/gi = 5498447/ug = Mm 21569/len = 395/STRA = for | | | | | | |
| 12 | 93970_at | Cluster Incl AI843085: UI-M-AK1-aes-b-10-0-UI s1 Mus musculus cDNA, 3 end/clone = UI-M-AK1-aes-b-10-0-UI/ | 186.9 | P | 425 | P | 327.2 | P |
| | | clone_end = 3/gb = AI843085/gi = 5477298/ug = Mm 21659/len = 279/STRA = for | | | | | | |
| 12 | 93976_at | Cluster Incl AI836686: UI-M-AP0-abh-h-07-0-UI s1 Mus musculus cDNA, 3 end/clone = UI-M-AP0-abh-h-07-0-UI/ | 372.9 | P | 785.1 | P | 587.8 | P |
| | | clone_end = 3/gb = AI836686/gi = 5470899/ug = Mm 21684/len = 371/STRA = for | | | | | | |
| 12 | 94008_at | Cluster Incl AW045358: UI-M-BH1-ako-c-05-0-UI s1 Mus musculus cDNA, 3 end/clone = UI-M-BH1-oko-c-05-0-UI/ | 216.9 | P | 313.3 | P | 220.9 | P |
| | | clone_end = 3/gb = AW045358/gi = 5905887/ug = Mm 21811/len = 296/STRA = for | | | | | | |
| 12 | 94014_at | Cluster Incl AA760073: vv69b08 r1 Mus musculus cDNA, 5 end/clone = IMAGE-1227639/clone_end = 5/gb = AA760073/ | 474.1 | P | 781.3 | P | 568.6 | P |
| | | gi = 2807867/ug = Mm 2184/len = 404/STRA = for | | | | | | |
| 12 | 94019_at | Cluster Incl AI852534 UI-M-BH0-alw-e-03-0-UI s1 Mus musculus cDNA, 3 end/clone = UI-M-BH0-aiw-e-03-0-UI/ | 426.6 | P | 723.7 | P | 618.5 | P |
| | | clone_end = 3/gb = AI852534/gi = 5496440/ug = Mm 21848/len = 265/STRA = for | | | | | | |
| 12 | 94242_at | Cluster Incl AA881309: vx11d11.r1 Mus musculus cDNA, 5 end/clone = IMAGE-1264149/clone_end = 5/gb = AA881309/ | 934.3 | P | 1219.7 | P | 856.8 | P |
| | | gi = 2990619/ug = Mm 22354/len = 461/STRA = for | | | | | | |
| 12 | 94253_at | Cluster Incl AW061243 UI-M-BH1-amj-c-11-0-UI s2 Mus musculus cDNA, 3 end/clone = UI-M-BH1-amj-c-11-0-UI/ | 308 | P | 521.7 | P | 486.5 | P |
| | | clone_end = 3/gb = AW061243/gi = 6008994/ug = Mm 22399/len = 430/STRA = for | | | | | | |
| 12 | 94281_at | Cluster Incl AW125138 UI-M-BH2 1-apz-c-01-0-UI s1 Mus musculus cDNA, 3 end/clone = UI-M-BH2 1-apz-c-01-0-UI/ | 328 | P | 453 | P | 343.4 | P |
| | | clone_end = 3/gb = AW125138/gi = 6100668/ug = Mm 22533/len = 352/STRA = for | | | | | | |
| 12 | 94322_at | Cluster Incl D42048 Mouse mRNA for squalene epoxidase, complete cds/cds = (619,2337)/gb = D42048/gi = 1217592/ | 534.5 | P | 1046.8 | P | 801.3 | P |
| | | ug = Mm.22663/len = 2611/STRA = for | | | | | | |
| 12 | 94325_at | Cluster Incl AW124932 UI-M-BH2 1-apu-g-09-0-UI s1 Mus musculus cDNA, 3 end/clone = UI-M-BH2.1-apu-g-09-0-UI/ | 515.2 | P | 1592 | P | 705.6 | P |
| | | clone_end = 3/gb = AW124932/gi = 6100462/ug = Mm 22675/len = 143/STRA = for | | | | | | |
| 12 | 94814_at | Cluster Incl AI957190 ul76g02.x1 Mus musculus cDNA, 3 end/clone = IMAGE-2136530/clone_end = 3/gb = AI957190/ | 976.5 | P | 1741.4 | P | 962.4 | P |
| | | gi = 5749899/ug = Mm 22704/len = 499/STRA = for | | | | | | |
| 12 | 94832_at | Cluster Incl U58105 Mus musculus Btk locus, alpha-D-galactosidase A (Ags), ribosomal protein (L44L), and Bruton a | 114.1 | P | 312.9 | P | 156.3 | P |
| | | tyrosine kinase (Btk) genes, complete cds/cds = (3,1352)/gb = U58105/gi = 1666699/ug = Mm 22757/len = 2150/STRA = for | | | | | | |
| 12 | 94865_at | Cluster Incl AW050059 UI-M-BH1-amm-e-04-0-UI s1 Mus musculus cDNA, 3 end/clone = UI-M-BH1-amm-e-04-0-UI/ | 149.9 | P | 236.7 | P | 184.6 | P |
| | | clone_end = 3/gb = AW050059/gi = 5910588/ug = Mm 23744/len = 335/STRA = for | | | | | | |
| 12 | 94903_at | Cluster Incl AI850801 UI-M-BG1-aii-f-04-0-UI s1 Mus musculus cDNA, 3 end/clone = UI-M-BG1-aii-f-04-0-UI/clone_end = 3/ | 673.8 | P | 1528.1 | P | 1247.1 | P |
| | | gb = AI850801/gi = 5494707/ug = Mm 24086/len = 547/STRA = for | | | | | | |
| 12 | 95408_at | Cluster Incl AW124487: UI-M-BH2 1-ape-h-10-0-UI.s1 Mus musculus cDNA, 3 end/clone = UI-M-BH2.1-ape-h-10-0-UI/ | 826.8 | P | 1133.9 | P | 762 | P |
| | | clone_end = 3/gb = AW124487/gi = 6099982/ug = Mm 24220/len = 214/STRA = for | | | | | | |
| 12 | 95431_at | Cluster Incl AA623426: vn33a01.r1 Mus musculus cDNA, 5 end/clone = IMAGE-1022952/clone_end = 5/gb = AA623426/ | 35.7 | P | 78.1 | P | 82.8 | P |
| | | gi = 2527302/ug = Mm 24463/len = 303/STRA = for | | | | | | |
| 12 | 95448_at | Cluster Incl AI839371 UI-M-AN0-acj-g-11-0-UI s1 Mus musculus cDNA, 3 end/clone = UI-M-AN0-acj-g-11-0-UI/ | 612.8 | P | 792.9 | P | 872.6 | P |
| | | clone_end = 3/gb = AI839371/gi = 5473584/ug = Mm 2462/len = 393/STRA = for | | | | | | |
| 12 | 95467_at | Cluster Incl AW120746 UI-M-BH2.3-any-c-11-0-UI.s1 Mus musculus cDNA, 3 end/clone = UI-M-BH2 3-any-c-11-0-UI/ | 179.8 | P | 299.5 | P | 151.8 | P |
| | | clone_end = 3/gb = AW120746/gi = 6096079/ug = Mm 24763/len = 416/STRA = for | | | | | | |
| 12 | 95508_at | Cluster Incl X61453: M musculus mRNA for H19 clone/cds = (0,1946)/gb = X61453/gi = 51135/ug = Mm 25203/len = 2092/ | 1104.9 | P | 1709.5 | P | 1295.7 | P |
| | | STRA = for | | | | | | |
| 12 | 96010_at | Cluster Incl AW047023: UI-M-BH1-alp-h-11-0-UI s1 Mus musculus cDNA, 3 end/clone = UI-M-BH1-alp-h-11-0-UI/ | 442.6 | P | 703.1 | P | 677.2 | P |
| | | clone_end = 3/gb = AW047023/gi = 5907552/ug = Mm 25548/len = 399/STRA = for | | | | | | |
| 12 | 96011_at | Cluster Incl AB009275 Mus musculus gene for matrin3, complete cds/cds = (0,920)/gb = AB009275/gi = 6497040/ | 97.6 | P | 306.7 | P | 146.9 | P |
| | | ug = Mm.25575/len = 921/STRA = for | | | | | | |
| 12 | 96012_f_at | Cluster Incl AI835367: UI-M-AQ0-aah-h-02-0-UI.s1 Mus musculus cDNA, 3 end/clone = UI-M-AQ0-aah-h-02-0-UI/ | 1881.3 | P | 2439.4 | P | 2117.8 | P |
| | | clone_end = 3/gb = AI835367/gi = 5469580/ug = Mm.25575/len = 407/STRA = for | | | | | | |
| 12 | 96013_l_at | Cluster Incl AI835367: UI-M-AQ0-aah-h-02-0-UI s1 Mus musculus cDNA, 3 end/clone = UI-M-AQ0-aah-h-02-0-UI/ | 227.7 | P | 345.3 | P | 225.4 | P |
| | | clone_end = 3/gb = AI835367/gi = 5469580/ug = Mm.25575/len = 407/STRA = for | | | | | | |
| 12 | 96019_at | Cluster Incl AI843476: UI-M-AQ1-aec-g-07-0-UI s1 Mus musculus cDNA,3 end/clone = UI-M-AQ1-aoc-g-07-0-UI/ | 1228.9 | P | 1546.5 | P | 1248.5 | P |
| | | clone_end = 3/gb = AI843476/gi = 5477889/ug = Mm.256999/len = 339/STRA = for | | | | | | |
| 12 | 96061_at | Cluster Incl AW060186: UI-M-BH1-amu-e-06-0-UI s1 Mus musculus cDNA, 3 end/clone = UI-M-BH1-amu-e-06-0-UI/ | 258.3 | P | 303.9 | P | 311.8 | P |
| | | clone_end = 3/gb = AW060186/gi = 6007952/ug = Mm 25699/len = 339/STRA = for | | | | | | |
| 12 | 96098_at | Cluster Incl AI848416: UI-M-AH1-ags-b-11-0-UI s1 Mus musculus cDNA,3 end/clone = UI-M-AH1-ags-b-11-0-UI/ | 182 | P | 290.5 | P | 216.2 | P |
| | | clone_end = 3/gb = AI848416/gi = 5492322/ug = Mm 26775/len = 499/STRA = for | | | | | | |
| 12 | 96628_at | Cluster Incl X54327: M musculus mRNA for glutamyl-tRNA synthetase/cds = UNKNOWN/gb = X54327/gi = 5804944/ | 278.9 | P | 392.3 | P | 255. 4 | P |
| | | ug = Mm 27190/len = 3528/STRA = for | | | | | | |
| 12 | 96647_at | Cluster Incl AA645440: vq66d12.s1 Mus musculus cDNA, 5 end/clone = IMAGE-1107287/clone_end = 5/gb = AA645440/ | 333.5 | P | 537.8 | P | 397.3 | P |
| | | gi = 2571869/ug = Mm 27293/len = 456/STRA = for | | | | | | |
| 12 | 96695_at | Cluster Incl AF089812 Mus musculus ubiquitin-conjugating enzyme HR6A mRNA, complete cds/cds = (128,586)/ | 141.9 | P | 201.3 | P | 131.2 | P |
| | | gb = AF089812/gi = 3769548/ug = Mm 27541/len = 1647/STRA = for | | | | | | |
| 12 | 97205_at | Cluster Incl AI848056 UI-M-AH1-agp-h-08-0-UI.s1 Mus musculus cDNA, 3 end/clone = UI-M-AH1-agp-h-08-0-UI/ | 576.8 | P | 1058.4 | P | 788.1 | P |
| | | clone_end = 3/gb = AI848056/gi = 5491962/ug = Mm 27695/len = 369/STRA = for | | | | | | |
| 12 | 97237_at | Cluster Incl AI837771 UI-M-AK0-adl-d-01-0-UI s1 Mus musculus cDNA, 3 end/clone = UI-M-AK0-adl-d-01-0-UI/ | 272.2 | P | 398 | P | 357.1 | P |
| | | clone_end = 3/gb = AI837771/gi = 5471989/ug = Mm 27831/len = 480/STRA = for | | | | | | |
| 12 | 97274_at | Cluster Incl Y13071 Mus musculus mRNA for 26S proteasome non-ATPase subunit/cds = (236,1165)/gb = Y13071/ | 765 | P | 1024.8 | P | 849.9 | P |
| | | gi = 2505939/ug = Mm 27933/len = 1479/STRA = for | | | | | | |
| 12 | 97292_at | Cluster Incl AI845225 UI-M-BG0-aht-f-07-0-UI.s1 Mus musculus cDNA, 3 end/clone = UI-M-BG0-aht-f-07-0-UI/ | 411 | P | 946.4 | P | 546.4 | P |
| | | clone_end = 3/gb = AI845225/gi = 5489131/ug = Mm 28026/len = 258/STRA = for | | | | | | |
| 12 | 97305_at | Cluster Incl AW123267 UI-M-BH2 1-apg-e-11-0-UI s1 Mus musculus cDNA, 3 end/clone = UI-M-BH2.1-apg-e-11-0-UI/ | 357.7 | P | 398.9 | P | 412.7 | P |
| | | clone_end = 3/gb = AW123267/gi = 6098797/ug = Mm 28053/len = 147/STRA = for | | | | | | |
| 12 | 98912_at | Cluster Incl AW047811 UI-M-BH1-als-a-04-0-UI s1 Mus musculus cDNA, 3 end/clone = UI-M-BH1-als-a-04-0-UI/ | 134.7 | P | 396.9 | P | 264.2 | P |
| | | clone_end = 3/gb = AW047811/gi = 5908340/ug = Mm 28600/len = 426/STRA = for | | | | | | |
| 12 | 98948_at | Cluster Incl AI785289 uj39f05 x1 Mus musculus cDNA, 3 end/clone = IMAGE-1922337/clone_end = 3/gb = AI785289/ | 17.9 | P | 200.2 | P | 150.5 | P |
| | | gi = 5333005/ug = Mm 28726/len = 523/STRA = for | | | | | | |
| 12 | 98956_at | Cluster/Incl AI844979 UI-M-BG0-ahu-c-07-0-UI s1 Mus musculus cDNA, 3 end/clone = UI-M-BG0-ahu-c-07-0-UI/ | 1000.4 | P | 1464.5 | P | 1418.6 | P |
| | | clone_end = 3/gb = AI844979/gi = 5488885/ug = Mm 28765/len = 245/STRA = for | | | | | | |
| 12 | 94462_at | Cluster Incl AW120719 UI-M-BH2 3-any-a-06-0-UI s1 Mus musculus cDNA, 3 end/clone = UI-M-BH2 3-any-a-06-0-UI/ | 319.2 | P | 473.6 | P | 458.1 | P |
| | | clone_end = 3/gb = AW120719/gi = 6096052/ug = Mm 28839/len = 482/STRA = for | | | | | | |
| 12 | 94471 _r_at | Cluster Incl AW045974 UI-M-BH1-alc-a-08-0-UI s1 Mus musculus cDNA, 3 end/clone = UI-M-BH1-alc-a-08-0-UI/ | 113.4 | P | 136.8 | P | 47.6 | P |
| | | clone_end = 3/gb = AW045974/gi = 5906503/ug = Mm 28851/len = 338/STRA = for | | | | | | |
| 12 | 94488_at | Cluster Incl AI847041 UI-M-AP1-agi-h-03-0-UI s1 Mus musculus cDNA, 3 end/clone = UI-M-AP1-agj-h-03-0-UI/ | 249.2 | P | 288.8 | P | 255.9 | P |
| | | clone_end = 3/gb = AI847041/gi = 5490947/ug = Mm 28899/len = 318/STRA = for | | | | | | |
| 12 | 95050_at | Cluster Incl AW124547 UI-M-BH2 1-apt-a-04-0-UI.s1 Mus musculus cDNA, 3 end/clone = UI-M-BH2 1-apl-a-04-0-UI/ | 335.6 | P | 483.2 | P | 458 | P |
| | | clone_end = 3/gb = AW124547/gi = 6100077/ug = Mm 29138/len = 468/STRA = rev | | | | | | |
| 12 | 95059_{— at} | Cluster Incl AW047399 UI-M-BH1-ame-g-05-0-UI s1 Mus musculus cDNA, 3 end/clone = UI-M-BH1-ame-g-05-0-UI/ | 531.4 | P | 738.7 | P | 1018 | P |
| | | clone_end = 3/gb = AW047399/gi = 5907928/ug = Mm 29159/len = 417/STRA = for | | | | | | |
| 12 | 95062_at | Cluster Incl AB026997 Mus musculus CAST mRNA for calpastatin, complete cds/cds = (249,2615)/gb = AB026997/ | 242.5 | P | 554.5 | P | 298.5 | P |
| | | gi = 6006274/ug = Mm.29163/len = 2622/STRA = for | | | | | | |
| 12 | 95158_at | Cluster IncI AF100956 Mus musculus major histocompatibility locus class II region, Fas-binding protein Daxx (DAXX) gene, | 488.1 | P | 833.3 | P | 803.6 | P |
| | | partial cds: Bing1 (BING1), lapasin (tapasin), RalGDS-like factor (RLF), KE2 (KE2), BING4 (BING4), beta1, 3-galactosyl | | | | | | |
| | | transferase (be/STRA = for | | | | | | |
| 12 | 95694_at | Cluster Incl X70956: M musculus T0P gene for topoisomerase I, exons 19-21/cds = (0,347)/gb = X70956/gi = 57932/ | 428.5 | P | 831.1 | P | 796.5 | P |
| | | ug = Mm 29663/len = 348/STRA = for | | | | | | |
| 12 | 95746_at | Cluster Incl AW123765 UI-M-BH2 1-apm-b-07-0-UI.s1 Mus musculus cDNA, 3 end/clone = UI-M-BH2.1-apm-b-07-0-UI/ | 899.4 | P | 1457.4 | P | 1220.1 | P |
| | | clone_end = 3/gb = AW123765/gi = 6099295/ug = Mm 29771/len = 402/STRA = for | | | | | | |
| 12 | 96269_at | Cluster Incl AA716963 vu69f10.r1 Mus musculus cDNA, 5 end/clone = IMAGE-1196883/clone_end = 5/gb = AA716963/ | 240 | P | 505.8 | P | 292.7 | P |
| | | gi = 2729237/ug = Mm 29847/len = 273/STRA = for | | | | | | |
| 12 | 96277_at | Cluster Incl AI850118 UI-M-BG1-aid-a-10-0-UI.s1 Mus musculus cDNA, 3 end/clone = UI-M-BG1-aid-a-10-0-UI/ | 302.9 | P | 475.5 | P | 366.9 | P |
| | | clone_end = 3/gb = AI850118/gi = 5494024/ug = Mm 29864/len = 311/STRA = for | | | | | | |
| 12 | 96289_at | Cluster Incl AW061287: UI-M-BH1-anw-b-03-0-UI s1 Mus musculus cDNA, 3 end/clone = UI-M-BH1-anw-b-03-0-UI/ | 424 | P | 648.9 | P | 582.3 | P |
| | | clone_end = 3/gb = AW061287/gi = 6009038/ug = Mm 29895/len = 336/STRA = for | | | | | | |
| 12 | 96331_at | Cluster Incl AI842754: UI-M-AP1-agi-a-10-0-UI.s1 Mus musculus cDNA, 3 end/clone = UI-M-AP1-agi-a-10-0-UI/ | 102.8 | P | 306.5 | P | 175.6 | P |
| | | clone_end = 3/gb = AI842754/gi = 5476967/ug = Mm 29971/len = 325/STRA = for | | | | | | |
| 12 | 96354_at | Cluster Incl AI854802: UI-M-BH0-aka-f-12-0-UI s1 Mus musculus cDNA, 3 end/clone = UI-M-BH0-aka-f-12-0-UI/ | 481.3 | P | 801.1 | P | 660.6 | P |
| | | clone_end = 3/gb = AI854802/gi = 5498708/ug = Mm 30007/len = 496/STRA = for | | | | | | |
| 12 | 96858_at | Cluster Incl AF100927 Mus musculus apoptosis-inducing factor AIF (Aif) mRNA, nuclear gene encoding mitochondrial | 305.3 | P | 507 | P | 442.5 | P |
| | | protein, complele cds/cds = (51,1889)/gb = AF100927/gi = 4323584/ug = Mm 30050/len = 1932/STRA = for | | | | | | |
| 12 | 96874_g_at | Cluster Incl AI843287: UI-M-AO1-aei-e-06-0-UI.s1 Mus musculus cDNA, 3 end/clone = UI-M-AO1-ael-e-06-0-UI/ | 1035.6 | P | 1964.3 | P | 1549.7 | P |
| | | clone_end = 3/gb = AI843287/gi = 5477500/ug = Mm 30069/len = 425/STRA = for | | | | | | |
| 12 | 96878_at | Cluster Incl AW048566: UI-M-BH1-alx-e-09-0-UI s1 Mus musculus cDNA, 3 end/clone = UI-M-BH1-aix-e-09-0-UI/ | 105.8 | P | 133.9 | P | 95.3 | P |
| | | clone_end = 3/gb = AW048566/gi = 5909095/ug = Mm 30073/len = 430/STRA = for | | | | | | |
| 12 | 96882_at | Cluster Incl AI842936: UI-M-AO1-aem-d-02-0-UI.s1 Mus musculus cDNA, 3 end/clone = UI-M-AO1-aem-d-02-0-UI/ | 1243.6 | P | 1865.1 | P | 1586.8 | P |
| | | clone_end = 3/gb = AI842936/gi = 5477149/ug = Mm.30078/len = 238/STRA = for | | | | | | |
| 12 | 96892_at | Cluster Incl AI836804 UI-M-AJ0-abc-h-09-0-UI s1 Mus musculus cDNA, 3 end/clone = UI-M-AJ0-abc-h-09-0-UI | 331.5 | P | 434.3 | P | 444.3 | P |
| | | clone_end = 3/gb = AI836804/gi = 5471017/ug = Mm 30097/len = 512/STRA = for | | | | | | |
| 12 | 96896_at | Cluster Incl AW214159: uo40f07.x1 Mus musculus cDNA, 3 end/clone = IMAGE-2645029/clone_end = 3/gb = AW214159/ | 943.9 | P | 2120.7 | P | 1626.3 | P |
| | | gi = 6520601/ug = Mm.30108/len = 217/STRA = for | | | | | | |
| 12 | 97471_at | Cluster Incl AI848442 UI-M-AH1-ags-e-10-0-UI s1 Mus musculus cDNA, 3 end/clone = UI-M-AH1-ags-e-10-0-UI/ | 322.9 | P | 383.3 | P | 382.7 | P |
| | | clone_end = 3/gb = AI848442/gi = 5492348/ug = Mm 30547/len = 212/STRA = for | | | | | | |
| 12 | 97505_at | Cluster Incl AI845999 UI-M-AP1-agi-e-02-0-UI s1 Mus musculus cDNA, 3 end/clone = UI-M-AP1-agi-e-02-0-UI/ | 647.5 | P | 891.3 | P | 710.7 | P |
| | | clone_end = 3/gb = AI845999/gi = 5489905/ug = Mm.31435/len = 386/STRA = for | | | | | | |
| 12 | 97511_at | Cluster Incl AI846600 UI-M-AQ1-aef-e-03-0-UI s1 Mus musculus cDNA, 3 end/clone = UI-M-AQ1-aef-e-03-0-UI/ | 325.2 | P | 613.9 | P | 523.2 | P |
| | | clone_end = 3/gb = AI846600/gi = 5490506/ug = Mm 31616/len = 436/STRA = for | | | | | | |
| 12 | 97514_at | Cluster Incl AW046438: UI-M-BH1-akp-d-11-0-UI s1 Mus musculus cDNA, 3 end/clone = UI-M-BH1-akp-d-11-0-UI/ | 171.8 | P | 232.1 | P | 191.9 | P |
| | | clone_end = 3/gb = AW046438/gi = 5906967/ug = Mm 31946/len = 438/STRA = rev | | | | | | |
| 12 | 97540_f_at | Cluster Incl M69069 Histocompatibility 2, D region locus 1/cds = (8,1042)/gb = M69069/gi = 199795/ug = Mm.33263/ | 527.1 | P | 1248.7 | P | 950.1 | P |
| | | len = 1379/STRA = rev | | | | | | |
| 12 | 98132_at | Cluster Incl X01756 Cytochrome c, somatic/cds = (0,317)/gb = X01756/gi = 50618/ug = Mm 35389/len = 318/STRA = for | 495.3 | P | 600.2 | P | 454.4 | P |
| 12 | 98136_at | Cluster Incl AF031486 Mus musculus apermidine aminopropyltransferase (Mapmsy) mRNA, complete cds/cds = (76,1176)/ | 149.8 | P | 261 | P | 319.6 | P |
| | | gb = AF031486/gi = 2625113/ug = Mm 3553/len = 1663/STRA = for | | | | | | |
| 12 | 98142_at | Cluster Incl AA930290 vs58d02 r1 Mus musculus cDNA, 5 end/clone = IMAGE-1150467/clone_end = 5/gb = AA930290/ | 187.4 | P | 265.1 | P | 246.7 | P |
| | | gi = 3079883/ug = Mm.35618/len = 285/STRA = for | | | | | | |
| 12 | 98572_at | Cluster Incl AW122551 UI-M-BH2.2-aox-d-09-0-UI s1 Mus musculus cDNA, 3 end/clone = UI-M-BH2 2-aox-d-09-0-UI/ | 128.1 | P | 299.4 | P | 241.1 | P |
| | | clone_end = 3/gb = AW122551/gi = 6098075/ug = Mm 37516/len = 480/STRA = for | | | | | | |
| 12 | 98594_at | Cluster Incl AW125453: UI-M-BH2.2-aqm-a-02-0-UI s1 Mus musculus cDNA, 3 end/clone = UI-M-BH2 2-aqm-a-02-0-UI/ | 103.5 | P | 309.2 | P | 144.3 | P |
| | | clone_end = 3/gb = AW125453/gi = 6100983/ug = Mm.38211/len = 290/STRA = for | | | | | | |
| 12 | 99107_at | Cluster Incl M31680: Growth hormone receptor/cds = (98,1072)/gb = M31680/gi = 193506/ug = Mm 3986/len = 1150/ | 123.7 | P | 190 | P | 210.8 | P |
| | | STRA = for | | | | | | |
| 12 | 99138_at | Cluster Incl AA756292 vv40e08 r1 Mus musculus cDNA, 5 end/clone = IMAGE-1224902/clone_end = 5/gb = AA756292/ | 271 | P | 307.2 | P | 256.4 | P |
| | | gi = 2803490/ug = Mm.41374/len = 369/STRA = rev | | | | | | |
| 12 | 99143_at | Cluster Incl AA614914: vo31go1 r1 Mus musculus cDNA, 5 end/clone = IMAGE-1051536/clone_end = 5/gb = AA614914/ | 278.7 | P | 403.5 | P | 304.8 | P |
| | | gi = 2502142/ug = Mm.4142/len = 375/STRA = for | | | | | | |
| 12 | 99180_at | Cluster Incl AI851237 UI-M-BH0-akf-a-05-0-UI s1 Mus musculus cDNA, 3 end/clone = UI-M-BH0-akf-a-05-0-UI/ | 538.2 | P | 879.8 | P | 551.7 | P |
| | | clone_end = 3/gb = AI851237/gi = 5495143/ug = Mm.41800/len = 421/STRA = for | | | | | | |
| 12 | 99196_at | Cluster Incl AI848532 UI-M-AP1-agf-f-07-0-UI a2 Mus musculus cDNA, 3 end/clone = UI-M-AP1-agf-07-0-UI/ | 639.3 | P | 936.9 | P | 777.6 | P |
| | | clone_end = 3/gb = AI848532/gi = 5492438/ug = Mm 41939/len = 378/STRA = for | | | | | | |
| 12 | 99579_at | Cluster Incl U59761: ATPase, Na+/K+beta 3 polypeptide/cds = (148,984)/gb = U59761/gi = 1762431/ug = Mm 424/len = 1842/ | 1497.1 | P | 2730 | P | 2310.5 | P |
| | | STRA = for | | | | | | |
| 12 | 100088_at | Cluster Incl M27073 Protein phosphatase 1, catalytic sutunit, beta isoform/cds = (95,1078)/gb = M27073/gi = 471977/ | 960.3 | P | 1494.9 | P | 964.7 | P |
| | | ug = Mm 4572/len = 2243/STRA = for | | | | | | |
| 12 | 100144_at | Cluster Incl X07699 Nucleolin/cds = (0,2123)/gb = X07699/gi = 53453/ug = Mm 4907/len = 2124/STRA = for | 1782.1 | P | 4241.3 | P | 3358.4 | P |
| 12 | 100560_at | Cluster Incl U95116 Platelet-activating factor acetylhydrolase, isoform 1b, beta 1 subunit/cds = (859,2091)/gb = U95116/ | 230.7 | P | 523.3 | P | 203.5 | P |
| | | gi = 2072302/ug = Mm.56337/len = 5832/STRA = for | | | | | | |
| 12 | 100568_at | Cluster Incl U90446: Mus musculus RNAse L Inhibitor (Mu-RLI) mRNA, complete cds/cds = (114,1913)/gb = U90446/ | 516.4 | P | 800.4 | P | 687.8 | P |
| | | gi = 3273416/ug = Mm.5831/len.= 3509/STRA = for | | | | | | |
| 12 | 101019_at | Cluster Incl U74683 Cathepsin C/cds = (67,1455)/gb = U74683/gi = 2145015/ug = Mm 684/len = 1767/STRA = for | 738.6 | P | 1148.3 | P | 870.7 | P |
| 12 | 101023_f_at | Cluster Incl AI843605 UI-M-AO1-aeh-h-06-0-UI s1 Mus musculus cDNA, 3 end/clone = UI-M-AO1-aeh-h-06-0-UI/ | 836.7 | P | 1398.3 | P | 997.6 | P |
| | | clone_end = 3/gb = AI843605/gi = 5477783/ug = Mm.6844/len = 379/STRA = for | | | | | | |
| 12 | 101035_at | Cluster Incl U35846 Mus musculus mRNA, complete cds/cds = (85,1599)/gb = U35846/gi = 2623754/ug = Mm 692/len = 3715/ | 248.4 | P | 373.4 | P | 393.5 | P |
| | | STRA = for | | | | | | |
| 12 | 101057_at | Cluster Incl AW121180 UI-M-BH2 3-aok-a-08-0-UI s1 Mus musculus cDNA, 3 end/clone = UI-M-BH2 3-aok-a-08-0-UI/ | 324.5 | P | 347.3 | P | 362.3 | P |
| | | clone_end = 3/gb = AW121180/gi = 6096513/ug = Mm 7066/len = 437/STRA = for | | | | | | |
| 12 | 101094_at | Cluster Incl AI836820 UI-M-AJ0-abd-c-01-0-UI s1 Mus musculus cDNA, 3 end/clone = UI-M-AJ0-abd-c-01-0-UI/ | 380.1 | P | 657.2 | P | 572.4 | P |
| | | clone_end = 3/gb = AI836820/gi = 5471033/ug = Mm.7381/1en = 357/STRA = for | | | | | | |
| 12 | 101097_at | Cluster Incl AW121176 UI-M-BH2 3-aok-a-03-0-UI.s1 Mus musculus cDNA, 3 end/clone = UI-M-BH2 3-aok-a-03-0-UI/ | 251.2 | P | 285.6 | P | 308.8 | P |
| | | clone_end = 3/gb = AW121176/gi = 6096509/ug = Mm.74084/len = 435/STRA = for | | | | | | |
| 12 | 101476_at | Cluster Incl U93050 Mus musculus poly(A) binding protein II (mPABII) gene, complete cds/cds = (0,908)/gb = U93050/ | 971.5 | P | 1161.6 | P | 817 | P |
| | | gi = 2351845/ug = Mm 7723/len = 909/STRA = for | | | | | | |
| 12 | 101489_at | Cluster Incl D12780 Mouse mRNA for S-adenosylmethionine decarboxylase, complete cds/cds = (326,1330)/gb = D12780/ | 270.8 | P | 581.7 | P | 560.7 | P |
| | | gi = 220330/ug = Mm 7880/len = 1497/STRA = for | | | | | | |
| 12 | 101518_at | Cluster Incl U38981 Mus musculus uterine mRNA, complete cds/cds = (205,603)/gb = U38981/gi = 1066284/ug = Mm 8486/ | 103.4 | P | 221.6 | P | 189.4 | P |
| | | len = 651/STRA = for | | | | | | |
| 12 | 101523_at | Cluster Incl AW046194: UI-M-BH1-akw-f-03-0-UI.s1 Mus musculus cDNA, 3 end/clone-UI-M-BH1-akw-f-03-0-UI/ | 2255.6 | P | 4273.1 | P | 2392.8 | P |
| | | clone_end = 3/gb = AW046194/gi = 5906723/ug = Mm 86187/len = 380/STRA = for | | | | | | |
| 12 | 101527_at | Cluster Incl M18209 Mouse transcription factor S-II, clone PSII-2/cds = (121,921)/gb = M18209/gi = 201936/ug = Mm 87235/ | 655.6 | P | 845.8 | P | 894.7 | P |
| | | len = 2466/STRA = for | | | | | | |
| 12 | 101528_at | Cluster Incl D00925 Mus musculus mRNA for transcription factor S-II-related proteins, complete cds/cds = UNKNOWN/ | 252.6 | P | 407.9 | P | 365.2 | P |
| | | gb = D00925/gi = 220590/ug = Mm.87235/len = 2257/STRA = for | | | | | | |
| 12 | 101529_g_at | Cluster Incl D00925 Mus musculus mRNA for transcription factor S-II-related proteins, complete cds/cds = UNKNOWN/ | 191.2 | P | 348.2 | P | 325.8 | P |
| | | gb = D00925/gi = 220590/ug = Mm.87235/len = 2257/STRA = for | | | | | | |
| 12 | 101548_at | Cluster Incl AI846345: UI-M-AN1-afa-c-08-0-UI s1 Mus musculus cDNA, 3 end/clone = UI-M-AN1-afa-c-08-0-UI/ | 109.3 | P | 166.4 | P | 123 | P |
| | | clone_end = 3/gb = AI846345/gi = 5490251/ug = Mm 88572/len = 394/STRA = rev | | | | | | |
| 12 | 101955_at | Cluster Incl AJ002387 Mus musculus mRNA for BiP/cds = (11,1978)/gb = AJ002387/gi = 2598561/ug = Mm 918/len = 2282/ | 589.3 | P | 1504.8 | P | 1086.7 | P |
| | | STRA = for | | | | | | |
| 12 | 102317_at | Cluster Incl AF061516 Mus musculus vesicle associated membrane protein 4 (Vamp4) mRNA, complete cds/cds = (77,502)/ | 265.8 | P | 377.2 | P | 310.4 | P |
| | | gb = AF061516/gi = 3108178/ug = Mm 10699/len = 819/STRA = for | | | | | | |
| 12 | 102786_at | Cluster Incl AI849432: UI-M-AJ1-ahc-f-04-0-UI.s1 Mus musculus cDNA, 3 end/clone = UI-M-AJ1-ahc-f-04-0-UI/ | 217.2 | P | 481.9 | P | 313.1 | P |
| | | clone_end = 3/gb = AI849432/gi = 5493338/ug = Mm.13796/len = 291/STRA = for | | | | | | |
| 12 | 103021_r_at | Cluster Incl AI317205: ui35g09 y1 Mus musculus cDNA 5 end/clone = IMAGE-1853344/clone_end = 5/gb = AI317205/ | 203.7 | P | 532 | P | 295.2 | P |
| | | gi = 4032472/ug = Mm 15918/len = 235/STRA = for | | | | | | |
| 12 | 103056_at | Cluster Incl AI849721 UI-M-AL1-ahn-b-07-0-UI s1 Mus musculus cDNA, 3 end/clone = UI-M-AL1-ahn-b-07-0-UI/ | 139.4 | P | 287.4 | P | 237.2 | P |
| | | clone_end = 3/gb = AI849721/gi = 5493627/ug = Mm 1654/len = 352/STRA = for | | | | | | |
| 12 | 103064_at | Cluster Incl AF016583: Mus musculus checkpoint kinase Chk1 (Chk1) mRNA, complete cds/cds = (22,1452)/gb = AF016583/ | 202.6 | P | 262.2 | P | 172 | P |
| | | gi = 2367670/ug = Mm 16753/len = 1962/STRA = for | | | | | | |
| 12 | 103074_f_at | Cluster Incl AI842969 UI-M-AO1-aem-g-05-0-UI s1 Mus musculus cDNA, 3 end/clone = UI-M-AO1-aem-g-05-0-UI/ | 382.3 | P | 574 | P | 364.1 | P |
| | | clone_end = 3/gb = AI842969/gi = 5477182/ug = Mm 16976/len = 340/STRA = for | | | | | | |
| 12 | 103086_at | Cluster Incl Y00208: Homeo box A5/cds = (74,886)/gb = Y00208/gi = 51371/ug = Mm 173/len = 1678/STRA = for | 159.3 | P | 243.4 | P | 199.1 | P |
| 12 | 103370_at | Cluster Incl AI848387 UI-M-AH1-ago-e-07-0-UI.s1 Mus musculus cDNA, 3 end/clone = UI-M-AH1-ago-e-07-0-UI/ | 386.9 | P | 1176.3 | P | 584 | P |
| | | clone_end = 3/gb = AI848367/gi = 5492268/ug = Mm 18555/len = 533/STRA = for | | | | | | |
| 12 | 103400_at | Cluster Incl D50523 Mouse mRNA for TI-227/cds = UNKNOWN/gb = D50523/gi = 1166394/ug = Mm 18728/len = 3577/ | 323.2 | P | 661.2 | P | 561.2 | P |
| | | STRA = for | | | | | | |
| 12 | 103654_at | Cluster Incl AB018374 Mus musculus GARP45 mRNA, complete cds/cds = (69,1289)/gb = AB018374/gi = 5263197/ | 256.7 | P | 585.7 | P | 284.4 | P |
| | | ug = Mm 20450/len = 1865/STRA = for | | | | | | |
| 12 | 103657_i_at | Cluster Incl AI836552 UI-M-AP0-abe-e-08-0-UI.s1 Mus musculus cDNA, 3 end/clone = UI-M-AP0-abe-e-08-0-UI/ | 115.7 | P | 209.7 | P | 164.2 | P |
| | | clone_end = 3/gb = AI836552/gi = 5470765/ug = Mm.20559/len = 417/STRA = for | | | | | | |
| 12 | 103874_r_at | Cluster Incl AA684456 vm64c06 s1 Mus musculus cDNA, 5 end/clone = IMAGE-1003018/clone_end = 5/gb = AA684456/ | 67.5 | P | 97.7 | P | 32.2 | P |
| | | gi = 2671042/ug = Mm.21173/len = 217/STRA = for | | | | | | |
| 12 | 103892_r_at | Cluster Incl AI197161 ue51h10 r1 Mus musculus cDNA, 5 end/clone = IMAGE-1494691/clone_end = 5/gb = AI197161/ | 189.6 | P | 273.5 | P | 210.7 | P |
| | | gi = 3749767/ug = Mm 21288/len = 475/STRA = for | | | | | | |
| 12 | 104046_at | Cluster Incl AI854141 UI-M-BH0-ajw-f-12-0-UI s1 Mus musculus cDNA, 3 end/clone = UI-M-BH0-ajw-f-12-0-UI/ | 482.6 | P | 694.2 | P | 529.7 | P |
| | | clone_end = 3/gb = AI854141/gi = 5498047/ug = Mm.21489/len = 577/STRA = for | | | | | | |
| 12 | 104080_at | Cluster Incl AW212479 uo89c10 x1 Mus musculus cDNA, 3 end/clone = IMAGE-2649714/clone_end = 3/gb = AW212479/ | 667.1 | P | 876.6 | P | 829.9 | P |
| | | gi = 6518566/ug = Mm 21753/len = 163/STRA = for | | | | | | |
| 12 | 104115_at | Cluster Incl AA867340: vx97a11 r1 Mus musculus cDNA, 5 end/clone = IMAGE-1293884/clone_end = 5/gb = AA867340/ | 158.2 | P | 589.5 | P | 632.7 | P |
| | | gi = 2962785/ug = Mm.21963/len = 308/STRA = for | | | | | | |
| 12 | 104303_i_at | Cluster Incl AW124151 UI-M-BH2 1-apr-f-08-0-UI s1 Mus musculus cDNA, 3 end/clone = UI-M-BH2.1-apr-f-08-0-UI/ | 53.9 | P | 141.1 | P | 122.1 | P |
| | | clone_end = 3/gb = AW124151/gi = 6099681/ug = Mm.22358/len = 298/STRA = for | | | | | | |
| 12 | 104304_r_at | Cluster Incl AW124151 UI-M-BH2 1-apr-f-08-0-UI s1 Mus musculus cDNA, 3 end/clone = UI-M-BH2.1-apr-f-08-0-UI/ | 163.5 | P | 192.9 | P | 150.7 | P |
| | | clone_end = 3/gb = AW124151/gi = 6099681/ug = Mm.22358/len = 298/STRA = for | | | | | | |
| 12 | 104531_at | Cluster Incl X60304 Protein kinase C, delta/cds = (13,2037)/gb = X60304/gi = 53436/ug = Mm 2314/len = 2538/STRA = rev | 823.9 | P | 1105.3 | P | 1300.5 | P |
| 12 | 104567_at | Cluster Incl AI848526 UI-M-AP1-agf-e-11-0-UI s2 Mus musculus cDNA, 3 end/clone = UI-M-AP1-agf-e-11-0-UI/ | 62.7 | P | 113.6 | P | 86.4 | P |
| | | clone_end = 3/gb = AI848526/gi = 5492432/ug = Mm 23880/len = 423/STRA = for | | | | | | |
| 12 | 104578_f_at | Cluster Incl AI195392 ue72102 r1 Mus musculus cDNA, 5 end/clone = IMAGE-1496667/clone_end = 5/gb = AI195392/ | 683.7 | P | 1546.4 | P | 1050.5 | P |
| | | gi = 3747998/ug = Mm 23961/len = 262/STRA = for | | | | | | |
| 12 | 104595_at | Cluster Incl AI846890 UI-M-AK1-aey-a-05-0-UI s1 Mus musculus cDNA, 3 end/clone = UI-M-AK1-aey-a-05-0-UI/ | 187.2 | P | 311 | P | 233.8 | P |
| | | clone_end = 3/gb = AI846890/gi = 5490796/ug = Mm.24025/len = 346/STRA = rev | | | | | | |
| 12 | 104738_at | Cluster Incl D63784 Mouse mRNA for MIDA1, complete cds/cds = (112,1977)/gb = D63784/gi = 1060924/ug = Mm 2467/ | 227.3 | P | 477.1 | P | 326.9 | P |
| | | len = 2037/STRA = for | | | | | | |
| 12 | 104745_at | Cluster Incl AA763874 vv48a12.r1 Mus musculus cDNA, 5 end/clone = IMAGE-1225630/clone_end = 5/gb = AA763874/ | 63.4 | P | 268.2 | P | 199.5 | P |
| | | gi = 2811396/ug = Mm.24719/len = 443/STRA = for | | | | | | |
| 12 | 104749_at | Cluster Incl AW123623 UI-M-BH2.1-api-e-02-0-UI s1 Mus musculus cDNA, 3 end/clone = UI-M-BH2.1-api-e-02-0-UI/ | 149.1 | P | 201.7 | P | 171.7 | P |
| | | clone_end = 3/gb = AW123623/gi = 6099153/ug = Mm 24765/len = 224/STRA = for | | | | | | |
| 12 | 94343_at | Cluster Incl AI604013 vr15c03 y1 Mus musculus cDNA, 5 end/clone = IMAGE-1120708/clone_end = 5/gb = AI604013/ | 134.9 | P | 263.5 | P | 183.8 | P |
| | | gi = 4613175/ug = Mm.25122/len = 164/STRA = for | | | | | | |
| 12 | 94361_at | Cluster Incl AI836083 UI-M-AJ0-abb-f-06-0-UI s1 Mus musculus cDNA, 3 end/clone = UI-M-AJ0-abb-f-06-0-UI/ | 426.5 | P | 691.1 | P | 579.3 | P |
| | | clone_end = 3/gb = AI836083/gi = 5470296/ug = Mm.25264/len = 529/STRA = for | | | | | | |
| 12 | 94985_at | Cluster Incl AI846392 UI-M-AN1-afa-h-02-0-UI s1 Mus musculus cDNA, 3 end/clone = UI-M-AN1-afa-h-02-0-UI/ | 216.7 | P | 464.5 | P | 343.8 | P |
| | | clone_end = 3/gb = AI846392/gi = 5490298/ug = Mm.27303/len = 399/STRA = for | | | | | | |
| 12 | 95522_i_at | Cluster Incl AB024005.Mus musculus mRNA for KRAB-containing zinc-finger protein KRAZ2, complete cds/ | 188.8 | P | 365.2 | P | 277.4 | P |
| | | cds = (307,2127)/gb = AB024005/gi = 4514560/ug = Mm.27575/len = 2247/STRA = for | | | | | | |
| 12 | 95530_at | Cluster Incl AW060250 UI-M-BH1-amv-e-11-0-UI s1 Mus musculus cDNA, 3 end/clone = UI-M-BH1-amv-e-11-0-UI/ | 137.3 | P | 230.6 | P | 163.4 | P |
| | | clone_end = 3/gb = AW060250/gi = 6008041/ug = Mm.27629/len = 581/STRA = for | | | | | | |
| 12 | 95594_at | Cluster Incl AI847486 UI-M-AI1-afs-g-07-0-UI.s1 Mus musculus cDNA, 3 end/clone = UI-M-AI1-afs-g-07-0-UI/clone_end = 3/ | 347.8 | P | 436.1 | P | 373.6 | P |
| | | gb = AI847486/gi = 5491392/ug = Mm 27914/len = 349/STRA = for | | | | | | |
| 12 | 95611_at | Cluster Incl AA726364: vu90e02 r1 Mus musculus cDNA, 5 end/clone = IMAGE-1209434/clone_end = 5/gb = AA726364/ | 880.7 | P | 1819.8 | P | 1883.1 | P |
| | | gi = 2744071/ug = Mm.27994/len = 374/STRA = for | | | | | | |
| 12 | 95613_at | Cluster Incl AW122573 UI-M-BH2 2-aox-f-07-0-UI s1 Mus musculus cDNA, 3 end/clone = UI-M-BH2 2-aox-f-07-0-UI/ | 331.7 | P | 570.8 | P | 512.3 | P |
| | | clone_end = 3/gb = AW122573/gi = 6098098/ug = Mm.28001/len = 448/STRA = rev | | | | | | |
| 12 | 96135_at | Cluster Incl AA833425: ub58d03 r1 Mus musculus cDNA, 5 end/clone = IMAGE-1381925/clone_end = 5/gb = AA833425/ | 687.8 | P | 1341.8 | P | 792.9 | P |
| | | gi = 2907153/ug = Mm.28149/len = 490/STRA = for | | | | | | |
| 12 | 96152_at | Cluster Incl AI645561: vt98h08 x1 Mus musculus cDNA, 3 end/clone = IMAGE-1179231/clone_end = 3/gb = AI645561/ | 207.7 | P | 509.4 | P | 372.9 | P |
| | | gi = 4724036/ug = Mm 28256/len = 332/STRA = for | | | | | | |
| 12 | 96755_at | Cluster Incl AA638002: vr30d10 r1 Mus musculus cDNA, 5 end/clone = IMAGE-1122163/clone_end = 5/gb = AA638002/ | 631.1 | P | 878.4 | P | 733.6 | P |
| | | gi = 2561590/ug = Mm 28982/len = 136/STRA = for | | | | | | |
| 12 | 96773_at | Cluster Incl AW125408: UI-M-BH2 3-aqh-d-12-0-UI s1 Mus musculus cDNA, 3 end/clone = UI-M-BH2 3-aqh-d-12-0-UI/ | 234.9 | P | 318 | P | 286.8 | P |
| | | clone_end = 3/gb = AW125408/gi = 6100938/ug = Mm 29036/len = 480/STRA = for | | | | | | |
| 12 | 96836_r_at | Cluster Incl AI447619: mr29c12.x1 Mus musculus cDNA, 3 end/clone = IMAGE-598870/clone_end = 3/gb = AI447619/ | 26.3 | P | 46.9 | P | 26.4 | P |
| | | gi = 4305257/ug = Mm.29434/len = 217/STRA = for | | | | | | |
| 12 | 97394_at | Cluster Incl AA794509: vr46h05 s1 Mus musculus cDNA, 5 end/clone = IMAGE-1123737/clone_end = 5/gb = AA794509/ | 126.5 | P | 279 | P | 212.4 | P |
| | | gi = 2857464/ug= Mm 29825/len = 571/STRA = for | | | | | | |
| 12 | 97947_at | Cluster Incl AI836959: UI-M-AJ0-aaz-b-08-0-UI s1 Mus musculus cDNA, 3 end/clone = UI-M-AJ0-aaz-b-08-0-UI/ | 266.2 | P | 632.6 | P | 379.2 | P |
| | | clone_end = 3/gb = AI836959/gi = 5471172/ug = Mm 3037/len = 449/STRA = for | | | | | | |
| 12 | 97991_at | Cluster Incl X02452: Mouse Ki-ras cellular oncogene exon 1 from Y1 adrenal tumor cells (and joined CDS)/cds = (11,577)/ | 128.7 | P | 302.3 | P | 223.1 | P |
| | | gb = X02452/gi = 52798/ug= Mm 31530/len = 1722/STRA = for | | | | | | |
| 12 | 98013_at | Cluster Incl AA666464: vm48a05.r1 Mus musculus cDNA, 5 end/clone = IMAGE-1001456/clone_end = 5/gb = AA666464/ | 126.7 | P | 236.2 | P | 203.6 | P |
| | | gi = 2625165/ug = Mm 3224/len = 164/STRA = for | | | | | | |
| 12 | 98438_f_at | Cluster Incl X16202 Mouse Q4 class 1 MHC gene (exon 5)/cds = (0,91)/gb = X16202/gi = 53862/ug = Mm 34421/len = 116/ | 723.3 | P | 1078.5 | P | 863.6 | P |
| | | STRA = for | | | | | | |
| 12 | 98441_at | Cluster Incl L23971 Fragile X mental retardation syndrome 1 homolog/cds = (123,1967)/gb = L23971/gi = 398044/ | 104.1 | P | 305.5 | P | 216.5 | P |
| | | ug = Mm.3451/len = 4240/STRA = rev | | | | | | |
| 12 | 98491_at | Cluster Incl AI286904: ui78d09 y1 Mus musculus cDNA, 5 end/clone = IMAGE-1888529/clone_end = 5/gb = AI286904/ | 171.9 | P | 276 | P | 216.8 | P |
| | | gi = 3926657/ug_Mm 35596/len = 352/STRA = for | | | | | | |
| 12 | 98509_at | Cluster Incl AI834979 UI-M-AM1-afw-e-03-0-UI s1 Mus musculus cDNA, 3 end/clone = UI-M-AM1-afw-e-03-0-UI/ | 345.5 | P | 458.7 | P | 306.6 | P |
| | | clone_end = 3/gb = AI834979/gi = 5469192/ug = Mm.3584/len = 451/STRA = for | | | | | | |
| 12 | 99462_at | Cluster Incl D38046 Mouse mRNA for typeII DNA topoisomerase beta isoform, complete cds/cds = (46,4884)/gb = D38046/ | 179.4 | P | 448.7 | P | 309.1 | P |
| | | gi = 533330/ug = Mm.4093/len = 5207/STRA = for | | | | | | |
| 12 | 99467_at | Cluster Incl AI846922.UI-M-AK1-aey-d-05-0-UI s1 Mus musculus cDNA, 3 end/clone = UI-M-AK1-aey-d-05-0-UI/ | 156.5 | P | 577.5 | P | 287.4 | P |
| | | clone_end = 3/gb = AI846922/gi = 5490828/ug = Mm 41224/len = 381/STRA = for | | | | | | |
| 12 | 99935_at | Cluster Incl D14340 Tight junction protein 1/cds = 403,5640)/gb = D14340/gi = 303709/ug = Mm 4342/len = 7046/STRA = for | 348.6 | P | 547.5 | P | 339.5 | P |
| 12 | 99949_at | Cluster Incl AW060324 UI-M-BH1-anh-a-06-0-UI s1 Mus musculus cDNA, 3 end/clone = UI-M-BH1-anh-a-06-0-UI/ | 34.9 | P | 172.6 | P | 166.6 | P |
| | | clone_end = 3/gb = AW060324/gi = 6008075/ug = Mm 43681/len = 475/STRA = for | | | | | | |
| 12 | 99959_at | Cluster Incl AW061337 UI-M-BH1-anw-f-11-0-UI.s1 Mus musculus cDNA, 3 end/clone = UI-M-BH1-anw-f-11-0-UI/ | 75.4 | P | 156 | P | 115.3 | P |
| | | clone_end = 3/gb = AW061337/gi = 6009088/ug = Mm 4410/len = 475/STRA = for | | | | | | |
| 12 | 99985_at | Cluster Incl AB027565 Mus musculus TXNRD1 mRNA for thioredoxin reductase 1, complete cds/cds = (158,1657)/ | 415.5 | P | 705.9 | P | 556.9 | P |
| | | gb = AB027565/gi = 6467192/ug= Mm 44552/len = 3328/STRA = for | | | | | | |
| 12 | 99990_at | Cluster Incl U28789 Mus musculus p53-associated cellular protein PACT mRNA, partial cds/cds = (0,4764)/gb = U28789/ | 235.4 | P | 904.6 | P | 649.5 | P |
| | | gi = 1546778/ug = Mm 4480/len = 5191/STRA = for | | | | | | |
| 12 | 100400_at | Cluster Incl AW124329.UI-M-BH2 1-apq-e-12-0-UI s1 Mus musculus cDNA, 3 end/clone = UI-M-BH2 1-apq-e-12-0-UI/ | 107.5 | P | 182.3 | P | 98.2 | P |
| | | clone_end = 3/gb = AW124329/gi = 6099859/ug = Mm 464/len = 236/STRA = for | | | | | | |
| 12 | 100464_at | Cluster Incl AI840585: UI-M-AM0-adq-a-06-0-UI s1 Mus musculus cDNA, 3 end/clone = UI-M-AM0-adq-a-06-0-UI/ | 156.6 | P | 249.6 | P | 275.1 | P |
| | | clone_end = 3/gb = AI840585/gi = 5474798/ug = Mm 491/len = 447/STRA = for | | | | | | |
| 12 | 100469_at | Cluster Incl D78642 Nuclear transcription factor-Y alpha/cds = UNKNOWN/gb = D78642/gi = 1304151/ug = Mm 4929/ | 121.2 | P | 181.6 | P | 174.4 | P |
| | | len = 2121/STRA = for | | | | | | |
| 12 | 100954_at | Cluster Incl AF057287 Mus musculus RAB/Rip protein mRNA, partial cds/cds = (0,983)/gb = AF057287/gi = 3063650/ | 151.7 | P | 311.4 | P | 208 | P |
| | | ug = Mm 6461/len = 1598/STRA = for | | | | | | |
| 12 | 100980_at | Cluster Incl U58512 Mus musculus Rho-associated, coiled-coil forming protein kinase p160 ROCK-1 mRNA, complete cds/ | 124.9 | P | 311.4 | P | 196.9 | P |
| | | cds = (0,4064)/gb = U58512/gi = 1514695/ug = Mm 6710/len = 4065/STRA = for | | | | | | |
| 12 | 101459_at | Cluster Incl L10410 Chromodomain helicase DNA binding protein 1/cds = (170,5305)/gb = L10410/gi = 455014/ug = Mm 8137/ | 289.6 | P | 355.1 | P | 381.6 | P |
| | | len = 5349/STRA = for | | | | | | |
| 12 | 101890_f_at | Cluster Incl U53208 Zuotin related factor 1/cds = (68,1612)/gb = U53208/gi = 1256831/ug = Mm 88678/len = 1995/STRA = for | 226.7 | P | 418.5 | P | 306.8 | P |
| 12 | 101894_s_at | Cluster Incl X71978 Fused toes/cds = (213,1091)/gb = X71978/gi = 311631/ug = Mm.88777/len = 1793/STRA = for | 170.3 | P | 285.9 | P | 217.7 | P |
| 12 | 102645_at | Cluster Incl AI155285.ud84b11 r1 Mus musculus cDNA, 5 end/clone = IMAGE-1477533/clone_end = 5/gb = AI155285/ | 53.4 | P | 171.1 | P | 105.3 | P |
| | | gi = 3683754/ug = Mm 13146/len = 325/STRA = for | | | | | | |
| 12 | 102647 _g_at | Cluster Incl AW122284 UI-M-BH2 2-aos-b-02-0-UI s1 Mus musculus cDNA, 3 end/clone = UI-M-BH2 2-aos-b-02-0-UI/ | 480 | P | 987.6 | P | 729.7 | P |
| | | clone_end = 3/gb = AW122284/gi = 8097765/ug = Mm 13148/len = 336/STRA = for | | | | | | |
| 12 | 103300_at | Cluster Incl AW124239 UI-M-BH2 1-aph-e-12-0-UI.s1 Mus musculus cDNA, 3 end/clone = UI-M-BH2.1-aph-e-12-0-UI/ | 303.8 | P | 525.8 | P | 444.7 | P |
| | | clone_end = 3/gb = AW124239/gi = 6099769/ug = Mm 21081/len = 279/STRA = for | | | | | | |
| 12 | 103309_at | Cluster Incl AI853977 UI-M-BH0-aiv-f-01-0-UI s1 Mus musculus cDNA, 3 end/clone = UI-M-BH0-aiv-f-01-0-UI/ | 302 | P | 355 | P | 293.7 | P |
| | | clone_end = 3/gb = AI853977/gi = 5497883/ug = Mm 21158/len = 265/STRA = for | | | | | | |
| 12 | 103459_at | Cluster Incl AW124544 UI-M-BH2 1-apo-h-11-0-UI s1 Mus musculus cDNA, 3 end/clone = UI-M-BH2.1-apo-h-11-0-UI/ | 312.7 | P | 739.3 | P | 468.2 | P |
| | | clone end = 3/gb = AW124544/gi = 6100074/ug = Mm.21688/len = 256/STRA = for | | | | | | |
| 12 | 103500_at | Cluster Incl AI553463: vw56g01 x1 Mus musculus cDNA, 3 end/clone = IMAGE-1247856/clone_end = 3/gb = AI553463/ | 150.9 | P | 340.4 | P | 232.8 | P |
| | | gi = 4485826/ug = Mm 22344/len = 457/STRA = rev | | | | | | |
| 12 | 103525_at | Cluster Incl AA981725: ua28h11 r1 Mus musculus cDNA, 5 end/clone = IMAGE-1348101/clone_end = 5/gb = AA981725/ | 151.9 | P | 258.8 | P | 302.7 | P |
| | | gi = 3160494/ug = Mm 2290/len = 433/STRA = for | | | | | | |
| 12 | 104163_at | Cluster Incl AA711002: vt52g08.r1 Mus musculus cDNA, 5 end/clone = IMAGE-1166750/clone_end = 5/gb = AA711002/ | 332.2 | P | 428 | P | 388.4 | P |
| | | gi = 2720920/ug = Mm 27130/len = 377/STRA = for | | | | | | |
| 12 | 104179_at | Cluster Incl AI788669 uk47h11 x1 Mus musculus cDNA, 3 end/clone = IMAGE-1972197/clone_end = 3/gb = AI788669/ | 111.7 | P | 267.5 | P | 199.3 | P |
| | | gi = 5336385/ug = Mm 27308/len = 503/STRA = for | | | | | | |
| 12 | 104212_at | Cluster Incl AA607767 vo08g02 r1 Mus musculus cDNA, 5 end/clone = IMAGE-1040882/clone_end = 5/gb = AA607767/ | 177.6 | P | 240.2 | P | 206.4 | P |
| | | gi = 2455202/ug = Mm 27763/len = 509/STRA = for | | | | | | |
| 12 | 104399_at | Cluster Incl AI850965 UI-M-BH0-akd-f-12-0-UI s1 Mus musculus cDNA, 3 end/clone = UI-M-BH0-akd-f-12-0-UI/ | 199.1 | P | 318.8 | P | 274.3 | P |
| | | clone_end = 3/gb = AI850965/gi = 5494871/ug = Mm 2866/len = 323/STRA = for | | | | | | |
| 12 | 104404_at | Cluster Incl AA693221 vr58c04 s1 Mus musculus cDNA, 5 end/clone = IMAGE-1124838/clone_end = 5/gb = AA693221/ | 135.4 | P | 248.9 | P | 204.3 | P |
| | | gi = 2694159/ug = Mm 28701/len = 453/STRA = for | | | | | | |
| 12 | 92243_at | Cluster Incl AA117417: mp90d10.r1 Mus musculus cDNA, 5 end/clone = IMAGE-576499/clone_end = 5/gb = AA117417/ | 386.5 | P | 506 | P | 536 | P |
| | | gi = 1672496/ug = Mm.34910/len = 494/STRA = for | | | | | | |
| 12 | 92282_at | Cluster lncl AI646301: vs77f11.x1 Mus musculus cDNA, 3 end/clone = IMAGE-1152333/clone_end = 3/gb = AI646301/ | 198.4 | P | 240.4 | P | 221.9 | P |
| | | gi = 4724776/ug = Mm 3702/len = 305/STRA = for | | | | | | |
| 12 | 92360_at | Cluster Incl U43900.Signal transducing adaptor molecule (SH3 domain and ITAM motif) 1/cds = (42,1688)/gb = U43900/ | 286.4 | P | 513.2 | P | 567.5 | P |
| | | gi = 1556460/ug = Mm 3965/len = 2747/STRA = for | | | | | | |
| 12 | 92480_f_at | Cluster Incl AB024004.Mus musculus mRNA for KRAB-containing zinc-finger protein KRAZ1, complete cds/ | 59 | P | 224 | P | 127.2 | P |
| | | cds = (116,2074)/gb = AB024004/gi = 4514558/ug = Mm.42140/len = 2191/STRA = for | | | | | | |
| 12 | 92487_at | Cluster Incl AB023419 Mus musculus mRNA for mSox7, complete cds/cds = (50,1192)/gb = AB023419/gi = 5103026/ | 390 | P | 592.1 | P | 649.8 | P |
| | | ug = Mm 42162/len = 3266/STRA = for | | | | | | |
| 12 | 93426_at | Cluster Incl AA199023 mU13a03 r1 Mus musculus cDNA, 5 end/clone = IMAGE-639244/clone_end = 5/gb = AA199023/ | 254.7 | P | 580 | P | 326.8 | P |
| | | gi = 1794518/ug = Mm 6488/len = 203/STRA = for | | | | | | |
| 12 | 93455_s_at | Cluster Incl X56848.M musculus mRNA for bone morphogenetic protein 4 (BMP-4)/cds = (356,1582)/gb = X56848/gi = 50180/ | 308.9 | P | 602.7 | P | 292.5 | P |
| | | ug = Mm.6813/len = 1785/STRA = for | | | | | | |
| 12 | 93471_at | Cluster Incl AI594427-vo35a08 y1 Mus musculus cDNA, 5 end/clone = IMAGE-1051882/clone_end = 5/gb = AI594427/ | 167.8 | P | 418.6 | P | 305.2 | P |
| | | gI = 4603475/ug = Mm 7033/len = 447/STRA = for | | | | | | |
| 12 | 93701_at | Cluster Incl AA982124.vx56f01.r1 Mus musculus cDNA, 5 end/clone = IMAGE-1279225/clone_end = 5/gb = AA982124/ | 167.5 | P | 330.6 | P | 241.6 | P |
| | | gi = 3160243/ug = Mm.86261/len = 474/STRA = for | | | | | | |
| 12 | 93868_at | Cluster Incl AL021127 Mus musculus cosmid CMX137 containing part of Nsdhl/cds = (0,578)/gb = AL021127/gi = 2769543/ | 115.2 | P | 217.4 | P | 143.8 | P |
| | | ug = Mm 87851/len = 579/STRA = for | | | | | | |
| 12 | 94110_f_at | Cluster Incl D10627.Mus musculus mRNA, for zinc finger protein, partial cds/cds = (0,1040)/gb = D10627/gi = 220636/ | 143.1 | P | 441.2 | P | 183 | P |
| | | ug = Mm 10509/len = 1041/STRA = for | | | | | | |
| 12 | 95282_at | Cluster Incl J04633 Mouse heat shock protein 86 mRNA, complete cds, and 28S ribosomal RNA, partial sequence/ | 944 | P | 2042 | P | 1183.6 | P |
| | | cds = (260,2461)/gb = J04633/gi = 194030/ug = Mm 1843/len = 2975/STRA = for | | | | | | |
| 12 | 100760_at | Cluster lncl X97986 M musculus mRNA for desmocollin type 1/cds = (151,2649)/gb = X97986/gi = 1707586/ug = Mm 909/ | 350.2 | P | 846.4 | P | 614.9 | P |
| | | len = 2987/STRA = for | | | | | | |
| 12 | 101634_at | Cluster Incl M33212 Mouse nucleolar protein N038 mRNA, complete cds/cds = (78,956)/gb = M33212/gi = 200010/ | 2683.1 | P | 3239.3 | P | 2407.4 | P |
| | | ug = Mm 6343/len = 1260/STRA = for | | | | | | |
| 12 | 92592_at | Cluster Incl M25558 Glycerolphosphate dehydrogenase 1, cytoplasmic adult/cds = (0,1049)/gb = M25558/gi = 193601/ | 403.1 | A | 655 | P | 646.7 | P |
| | | ug = Mm 10669/len = 1050/STRA = for | | | | | | |
| 12 | 92871_at | Cluster Incl AW121840: UI-M-BH2 3-aoi-a-03-0-UI s1 Mus musculus cDNA, 3 end/clone = UI-M-BH2 3-aoi-a-03-0-UI/ | 204.6 | A | 351.3 | P | 254.4 | P |
| | | clone_end = 3/gb = AW121840/gi = 6097214/ug = Mm 14352/len = 466/STRA = for | | | | | | |
| 12 | 95416_at | Cluster Incl AI642184.uc46h07.x1 Mus musculus cDNA, 3 end/clone = IMAGE-1401085/clone_end = 3/gb = AI642184/ | 173.7 | M | 282 | P | 283.8 | P |
| | | gi = 4720659/ug = Mm.24280/len = 642/STRA = for | | | | | | |
| 12 | 97273_at | Cluster Incl AI845953: UI-M-AK1-aex-h-04-0-UI s1 Mus musculus cDNA, 3 end/clone = UI-M-AK1-aex-h-04-0-UI/ | 208.2 | P | 613.1 | P | 534.1 | P |
| | | clone_end = 3/gb = AI845953/gi = 5489859/ug = Mm 27932/len = 357/STRA = for | | | | | | |
| 12 | 96861_at | Cluster Incl AI852376.UI-M-BH0-ajo-e-04-0-UI.s1 Mus musculus cDNA, 3 end/clone = UI-M-BH0-ajo-e-04-0-UI/ | 160.9 | P | 204.2 | P | 191 | P |
| | | clone_end = 3/gb = AI852376/gi = 5496282/ug = Mm 30052/len = 348/STRA = for | | | | | | |
| 12 | 97506_at | Cluster Incl AI043016 uc87a04.x1 Mus musculus cDNA, 3 end/clone = IMAGE-1432590/clone_end = 3/gb = AI043016/ | 150.5 | P | 292.8 | P | 197 | P |
| | | gi = 3286276/ug = Mm 31512/len = 532/STRA = for | | | | | | |
| 12 | 98135_r_at | Cluster Incl AI851008.UI-M-BH0-ajv-c-01-0-UI.s1 Mus musculus cDNA, 3 end/clone = UI-M-BH0-ajv-c-01-0-UI/ | 36.7 | P | 58.4 | P | 23.5 | A |
| | | clone_end = 3/gb = AI851008/gi = 5494914/ug = Mm.35492/len = 438/STRA = for | | | | | | |
| 12 | 99662_at | Cluster Incl AI194767 ui56d10.x1 Mus musculus cDNA, 3 end/clone = IMAGE-1886419/clone_end = 3/gb = AI194767/ | 50.5 | P | 91.7 | P | 170.6 | P |
| | | gi = 3747373/ug = Mm 4379/len = 525/STRA = for | | | | | | |
| 12 | 101020_at | Cluster Incl AI842667 UI-M-AO1-aeo-a-04-0-UI.s1 Mus musculus cDNA, 3 end/clone = UI-M-AO1-aeo-a-04-0-UI/ | 105.4 | P | 195 | P | 137.5 | P |
| | | clone_end = 3/gb = AI842667/gi = 5476880/ug = Mm.684/len = 284/STRA = for | | | | | | |
| 12 | 101079_at | Cluster Incl AF093140 Mus musculus tip associating protein (Tap) mRNA, complete cds/cds = (105,1961)/gb = AF093140/ | 109 | P | 208.8 | P | 126.6 | P |
| | | gi = 3719434/ug = Mm 7271/len = 2034/STRA = for | | | | | | |
| 12 | 103063_at | Cluster Incl Z67747 M. musculus mRNA for ZT3 zinc finger factor/cds = (0,1570)/gb = Z67747/gi = 1061193/ug = Mm 16650/ | 42.7 | P | 169.7 | P | 85.5 | P |
| | | len = 2547/STRA = for | | | | | | |
| 12 | 103598_at | Cluster Incl U91922: D-E-A-D (aspartate-glutamate-alanine-aspartate) box polypeptide 9/cds = (107,4249)/gb = U91922/ | 107.5 | P | 287.4 | P | 206.2 | P |
| | | gi = 2961455/ug = Mm 20000/len = 4498/STRA = for | | | | | | |
| 12 | 103665_at | Cluster Incl AW122523 UI-M-BH2.2-aox-b-05-0-UI s1 Mus musculus cDNA, 3 end/clone = UI-M-BH2 2-aox-b-05-0-UI/ | 154.1 | M | 332.4 | P | 238.1 | P |
| | | clone_end = 3/gb = AW122523/gi = 6098047/ug = Mm 20697/len = 423/STRA = for | | | | | | |
| 12 | 104298_at | Cluster Incl AI842544 UI-M-AQ1-aea-e-05-0-UI.s1 Mus musculus cDNA, 3 end/clone = UI-M-AQ1-aea-e-05-0-UI/ | 117.8 | P | 124.3 | P | 125.4 | P |
| | | clone end = 3/gb = AI842544/gi = 5476757/ug = Mm 22337/len = 457/STRA = for | | | | | | |
| 12 | 104536_at | Cluster Incl U60530 MAD homolog 2, (Drosophila)/cds = (59,1462)/gb = U60530/gi = 1407781/ug = Mm 2334/len = 1639/ | 152.2 | P | 288.1 | P | 301.1 | P |
| | | STRA = for | | | | | | |
| 12 | 95521_s_at | Cluster Incl AB024005 Mus musculus mRNA for KRAB-containing zinc-finger protein KRAZ2, complete cds/ | 98.9 | P | 196.7 | P | 125.5 | P |
| | | cds = (307,2127)/gb = AB024005/gi = 4514560/ug = Mm 27575/len = 2247/STRA = for | | | | | | |
| 12 | 100459_at | Cluster Incl U66887: RAD50 homolog (S cerevisiae)/cds = (242,4180)/gb = U66887/gi = 1575574/ug = Mm 4888/len = 5088/ | 77 | P | 214.9 | P | 162.6 | P |
| | | STRA = for | | | | | | |
| 12 | 102872_f_at | Cluster Incl X74855 Zinc finger protein 51/cds = (350,2482)/gb = X74855/gi = 488832/ug = Mm 14812/len = 2640/STRA = for | 28.7 | A | 156.7 | P | 52 | P |
| 12 | 102887_at | Cluster Incl U94331 Mus musculus osteoprotegerin (OPG) mRNA, complete cds/cds = (90,1295)/gb = U94331/gi = 2072182/ | 145.2 | P | 418.3 | P | 414.2 | M |
| | | ug = Mm 15383/len = 1325/STRA = for | | | | | | |
| 12 | 103745_at | Cluster Incl AW227027.um65d05 y1 Mus musculus cDNA, 5 end/clone = IMAGE-2286249/clone_end = 5/gb = AW227027/ | 85.4 | A | 192.2 | P | 133.8 | P |
| | | gi = 6556323/ug = Mm.24475/len = 457/STRA = for | | | | | | |
| 12 | 104419_at | Cluster Incl AI132380 ue24a03.x1 Mus musculus cDNA, 3 end/clone = IMAGE-1481260/clone_end = 3/gb = AI132380/ | −6.7 | A | 123.5 | P | 43.2 | P |
| | | gi = 3602396/ug = Mm.28928/len = 441/STRA = for | | | | | | |
| 12 | 104501_at | Cluster Incl AI849048 UI-M-AH1-agw-b-07-0-UI.s1 Mus musculus cDNA, 3 end/clone = UI-M-AH1-agw-b-07-0-UI/ | 17.5 | M | 112.7 | P | 106.1 | P |
| | | clone_end = 3/gb = AI849048/gi = 5492954/ug = Mm 3055/len = 421/STRA = rev | | | | | | |
| 12 | 92408_at | Cluster Incl AI891514: ul59e08.x1 Mus musculus cDNA, 3 end/clone = IMAGE-2123366/clone_end = 3/gb = AI891514/ | 53.1 | P | 216 | P | 110.3 | P |
| | | gi = 5597416/ug = Mm.41046/len = 479/STRA = for | | | | | | |
| 12 | 92649_at | Cluster Incl Y18505: Mus musculus mRNA for XAP-5 protein/cds = (147,1166)/gb = Y18505/gi = 5327064/ug = Mm.4370/ | 163.1 | P | 264.5 | P | 203.5 | P |
| | | len = 1348/STRA = for | | | | | | |
| 12 | 95289_f_at | Cluster Incl AA189811: mt97g08 r1 Mus musculus cDNA, 5 end/clone = IMAGE-637886/clone_end = 5/gb = AA189811/ | 29.9 | A | 53.7 | P | 33.2 | P |
| | | gi = 1776416/ug = Mm.1882/len = 365/STRA = for | | | | | | |
| 12 | 98761_i_at | Cluster Incl L20450 Mus musculus DNA-binding protein mRNA, complete cds/cds = (93,1733)/gb = L20450/gi = 437443/ | −8.4 | A | 318.5 | P | 91.4 | P |
| | | ug = Mm 4596/len = 2768/STRA = for | | | | | | |
| 12 | 101294_g_at | Cluster Incl Z84471 M musculus G6pd-2 gene/cds = (0,1541)/gb = Z84471/gi = 1806125/ug = Mm 56918/len = 1542/ | 158.9 | P | 333.4 | P | 136.7 | P |
| | | STRA = for | | | | | | |
| 12 | 93568_i_at | Cluster Incl AI853444 UI-M-BH0-ajy-d-09-0-UI s1 Mus musculus cDNA, 3 end/clone = UI-M-BH0-ajy-d-09-0-UI/ | 67.3 | P | 98.6 | P | 11.1 | A |
| | | clone_end = 3/gb = AI853444/gi = 5497350/ug = Mm 204/len = 363/STRA = for | | | | | | |
| 12 | 94009_at | Cluster Incl AI848888 UI-M-AJ1-ahb-g-12-0-UI s1 Mus musculus cDNA, 3 end/clone = UI-M-AJ1-ahb-g-12-0-UI/ | −170.3 | P | 11.2 | P | 76.2 | P |
| | | clone_end = 3/gb = AI848888/gi = 5492794/ug = Mm.21811/len = 365/STRA = for | | | | | | |
| 12 | 94917_at | Cluster Incl AI844932 UI-M-BG0-ahv-f-12-0-UI s1 Mus musculus cDNA, 3 end/clone = UI-M-BG0-ahv-f-12-0-UI/ | 74.8 | M | 124.4 | P | 75 | P |
| | | clone_end = 3/gb = AI844932/gi = 5488838/ug = Mm 24156/len = 407/STRA = for | | | | | | |
| 12 | 95483_at | Cluster Incl AW123318 UI-M-BH2.1-apf-b-03-0-UI.s1 Mus musculus cDNA, 3 end/clone = UI-M-BH2 1-apf-b-03-0-UI/ | 218.1 | P | 427.9 | P | 422.1 | P |
| | | clone_end = 3/gb = AW123318/gi = 6098848/ug = Mm 24874/len = 371/STRA = for | | | | | | |
| 12 | 96052_at | Cluster Incl AI852457 UI-M-BH0-aip-e-04-0-UI s1 Mus musculus cDNA, 3 end/clone = UI-M-BH0-aip-e-04-0-UI/ | 219 | P | 291.8 | P | 245.8 | P |
| | | clone_end = 3/gb = AI852457/gi = 5496363/ug = Mm 26166/len = 426/STRA = for | | | | | | |
| 12 | 97243_at | Cluster Incl U74079 Mus musculus protein co-factor mRNA, complete cds/cds = (137,1204)/gb = U74079/gi = 1644403/ | 560.7 | A | 1019 | P | 669.6 | P |
| | | ug = Mm 27842/len = 1901/STRA = for | | | | | | |
| 12 | 98959_at | Cluster Incl AW120623 UI-M-BH2.3-anx-h-09-0-UI s1 Mus musculus cDNA, 3 end/clone = UI-M-BH2 3-anx-h-09-0-UI/ | 190.6 | P | 239.3 | P | 292.4 | P |
| | | clone_end = 3/gb = AW120623/gi = 6095956/ug = Mm 28773/len = 308/STRA = for | | | | | | |
| 12 | 94526_at | Cluster Incl AI848453 UI-M-AH1-ags-f-11-0-UI s1 Mus musculus cDNA, 3 end/clone = UI-M-AH1-ags-f-11-0-UI/ | 119.4 | P | 302.4 | A | 296.3 | P |
| | | clone_end = 3/gb = AI848453/gi = 5492359/ug = Mm.29032/len = 495/STRA = for | | | | | | |
| 12 | 98539_at | Cluster Incl AF071312 Mus musculus COP9 complex subunit 2 (COPS2) mRNA, complete cds/cds = (12,1334)/ | 31.4 | A | 118.6 | P | 53.6 | P |
| | | gb = AF071312/gi = 3309165/ug = Mm 3596/len = 1929/STRA = for | | | | | | |
| 12 | 103321_at | Cluster Incl AI536457 vu57h06 x1 Mus musculus cDNA, 3 end/clone = IMAGE-1195547/clone_end = 3/gb = AI536457/ | 40.9 | A | 199.2 | P | 84.8 | P |
| | | gi = 4450592/ug = Mm 17873/len = 435/STRA = for | | | | | | |
| 12 | 104285_at | Cluster Incl M62766 3-hydroxy-3-methylglutaryl-coenzyme A reductase/cds = (0,674)/gb = M62766/gi = 193881/ | 47 | A | 246.1 | P | 157.8 | P |
| | | ug = Mm 2226/len = 1045/STRA = for | | | | | | |
| 12 | 98966_at | Cluster Incl L42996 Mus musculus nuclear-encoded mitochondrial acyltransferase mRNA, complete cds/cds = (15,1463)/ | −13.8 | A | 134.4 | P | 24.2 | P |
| | | gb = L42996/gi = 1100214/ug = Mm 3636/len = 2664/STRA = for | | | | | | |
| 12 | 100512_at | Cluster Incl AI838853: UI-M-AN0-ack-b-11-0-UI s1 Mus musculus cDNA, 3 end/clone = UI-M-AN0-ack-b-11-0-UI/ | 58.7 | A | 295.6 | P | 210.2 | P |
| | | clone_end = 3/gb = AI838853/gi = 5473066/ug = Mm 52067/len = 397/STRA = for | | | | | | |
| 12 | 102197_at | Cluster Incl AJ222586 Mus musculus mRNA for NEFA protein, complete cds/cds = (287,1549)/gb = AJ222586/gi = 2612796/ | 24.1 | A | 76.9 | P | 50.2 | P |
| | | ug = Mm.9901/len = 1655/STRA = for | | | | | | |
| 12 | 103260_at | Cluster Incl AI838553: UI-M-AL0-abp-d-10-0-UI s1 Mus musculus cDNA, 3 end/clone = UI-M-AL0-abp-d-10-0-UI/ | 24 | A | 138.3 | P | 88.9 | P |
| | | clone_end = 3/gb = AI838553/gi = 5472731/ug = Mm 208/len = 491/STRA = rev | | | | | | |
| 12 | 95318_at | Cluster Incl AF045565 Zinc finger protein 105/cds = (231,1805)/gb = AF045565/gi = 3483132/ug = Mm 20430/len = 2002/ | 187.8 | M | 266.1 | P | 221.3 | P |
| | | STRA = for | | | | | | |
| 12 | 96560_at | Cluster Incl AA648027 vn68h05 r1 Mus musculus cDNA, 5 end/clone = IMAGE-1026393/clone_end = 5/gb = AA648027/ | 490.3 | A | 521.4 | P | 457.2 | P |
| | | gi = 2574456/ug = Mm 28931/len = 419/STRA = for | | | | | | |
| 12 | 99828_at | Cluster Incl AA673252 vp49C07 r1 Mus musculus cDNA, 3 end/clone = IMAGE-1080012/clone_end = 3/gb = AA673252/ | 135.1 | P | 166.9 | P | 158.9 | P |
| | | gi = 2646534/ug = Mm 6148/len = 370/STRA = rev | | | | | | |
| 12 | 93502_r_at | Cluster Incl AF058955 Mus musculus ATP-specific succinyl-CoA synthetase beta subunit (Scs) mRNA, partial cds/ | 116.4 | A | 222.2 | P | 156.1 | P |
| | | cds = (0,1280)/gb = AF058955/gi = 3766200/ug = Mm 19154/len = 1991/STRA = for | | | | | | |
| 12 | 95660_at | Cluster Incl AI851815: UI-M-BH0-ais-c-07-0-UI s1 Mus musculus cDNA, 3 end/clone = UI-M-BH0-ais-c-07-0-UI/ | −27.2 | A | 227.3 | P | 132.9 | M |
| | | clone_end = 3/gb = AI851815/gi = 5495721/ug = Mm 29553/len = 341/STRA = for | | | | | | |
| 12 | 98073_at | Cluster Incl U66249 Mus musculus Cut alternatively spliced product (CASP) mRNA, partial cds/cds = (0,957)/gb = U66249/ | 30.9 | A | 285.7 | P | 206.6 | P |
| | | gi = 1518859/ug = Mm.34465/len = 1757/STRA = for | | | | | | |
| 12 | 101524_at | Cluster Incl AI851595 UI-M-BH0-aki-h-08-0-UI.s1 Mus musculus cDNA, 3 end/clone = UI-M-BH0-aki-h-08-0-UI/ | 3.9 | P | 81.1 | P | 11.7 | A |
| | | clone_end = 3/gb = AI851595/gi = 5495501/ug = Mm 86187/len = 521/STRA = for | | | | | | |
| 12 | 102982_at | Cluster Incl AI854581 UI-M-BH0-ake-a-06-0-UI.s1 Mus musculus cDNA, 3 end/clone = UI-M-BH0-ake-a-06-0-UI/ | 83.2 | A | 324.5 | P | 205.2 | P |
| | | clone_end = 3/gb = AI854581/gi = 5498487/ug = Mm 1511/len = 562/STRA = for | | | | | | |
| 12 | 96148_at | Cluster Incl AI844238 UI-M-AL1-ahk-f-10-0-UI s1 Mus musculus cDNA, 3 end/clone = UI-M-AL1-ahk-f-10-0-UI/ | 117.8 | A | 176.2 | P | 127.4 | P |
| | | clone_end = 3/gb = AI844238/gi = 5488144/ug = Mm 28232/len = 395/STRA = rev | | | | | | |
| 12 | 96165_at | Cluster Incl AW125109 UI-M-BH2.1-apx-h-05-0-UI s1 Mus musculus cDNA, 3 end/clone = UI-M-BH2 1-apx-h-05-0-UI/ | 117.3 | A | 285 | P | 309.3 | P |
| | | clone_end = 3/gb = AW125109/gi = 6100639/ug = Mm.28370/len = 376/STRA = rev | | | | | | |
| 12 | 99923_at | Cluster Incl AA615429: vo78b10.r1 Mus musculus cDNA, 5 end/clone = IMAGE-1065211/clone_end = 5/gb = AA615429/ | 46.9 | P | 91.4 | P | 46 | P |
| | | gi = 2502657/ug = Mm.43157/len = 375/STRA = for | | | | | | |
| 12 | 104418_at | Cluster Incl AA796868: vp19h01.r1 Mus musculus cDNA, 5 end/clone = IMAGE-1069105/clone_end = 5/gb = AA796868/ | −32 | A | 90 | P | 36 | P |
| | | gi = 2859823/ug = Mm 28920/len = 343/STRA = for | | | | | | |
| 12 | 93175_at | Cluster Incl AI049206 ub39g05 r1 Mus musculus cDNA, 5 end/clone = IMAGE-1380152/clone_end = 5/gb = AI049206/ | 210.8 | P | 267.4 | P | 200 | A |
| | | gi = 3297493/ug = Mm 5357/len = 501/STRA = for | | | | | | |
| 12 | 92806_at | Cluster Incl AI843868 UI-M-AK1-aeu-d-04-0-UI.s1 Mus musculus cDNA, 3 end/clone = UI-M-AK1-aeu-d-04-0-UI/ | 260.5 | A | 392.4 | P | 347.4 | A |
| | | clone_end = 3/gb = AI843868/gi = 5478081/ug = Mm 12746/len = 408/STRA = for | | | | | | |
| 12 | 93569_f_at | Cluster Incl AI853444 UI-M-BH0-ajy-d-09-0-UI s1 Mus musculus cDNA, 3 end/clone = UI-M-BH0-ajy-d-09-0-UI/ | 38.4 | P | 61 | P | 21.4 | A |
| | | clone_end = 3/gb = AI853444/gi = 5497350/ug = Mm.204/len = 363/STRA = for | | | | | | |
| 12 | 94239_at | Cluster Incl Y08701 M. musculus mRNA for Pinin protein/cds = (18,2195)/gb = Y08701/gi = 2570048/ug = Mm 22347/ | 30 | A | 153.4 | P | 18.2 | A |
| | | len = 2813/STRA = for | | | | | | |
| 12 | 98944_at | Cluster Incl AI848343: UI-M-AH1-ago-c-12-0-U1.s1 Mus musculus cDNA, 3 end/clone = UI-M-AH1-ago-c-12-0-UI/ | 38.1 | A | 276.8 | P | 176.7 | P |
| | | clone_end = 3/gb = AI848343/gi = 5492249/ug = Mm.28704/len = 491/STRA = for | | | | | | |
| 12 | 103346_at | Cluster Incl AF033564: Mus musculus cdc2/CDC28-like kinase 2 (Clk2) mRNA, complete cds/cds = (24,1523)/ | 238.4 | P | 299.6 | M | 255.2 | P |
| | | gb = AF033564/gi = 2645851/ug = Mm 18268/len = 1538/STRA = rev | | | | | | |
| 12 | 104088_at | Cluster Incl AW060311 UI-M-BH1-amv-d-09-0-UI s1 Mus musculus cDNA, 3 end/clone = UI-M-BH1-amv-d-09-0-UI/ | 151.9 | A | 333.9 | P | 231.9 | A |
| | | clone_end = 3/gb = AW060311/gi = 6008027/ug = Mm.21795/len = 443/STRA = for | | | | | | |
| 12 | 104112_at | Cluster Incl AI854033 UI-M-BH0-ajm-c-10-0-UI.s1 Mus musculus cDNA, 3 end/clone = UI-M-BH0-ajm-c-10-0-UI/ | 115.1 | A | 223.7 | M | 176.9 | P |
| | | clone_end = 3/gb = AI854033/gi = 5497939/ug = Mm.21936/len = 468/STRA = for | | | | | | |
| 12 | 100988_at | Cluster Incl AA796690 vp15a12 rt Mus musculus cDNA, 5 end/clone = IMAGE-1068670/clone_end = 5/gb = AA796690/ | 62 | P | 192.9 | P | 226.1 | A |
| | | gi = 2859645/ug = Mm 6807/len = 403/STRA = for | | | | | | |
| 12 | 102678_at | Cluster Incl L27990 Mus musculus Ro protein mRNA, complete cds/cds = (140,1552)/gb = L27990/gi = 825145 ug = Mm 1384/ | 178.5 | A | 260.3 | P | 222.1 | P |
| | | len = 1840/STRA = for | | | | | | |
| 12 | 104216_at | Cluster Incl AF068921 Mus musculus Ras-binding protein SUR-8 mRNA, complete cds/cds = (0,1748)/gb = AF068921/ | 73.3 | A | 183.3 | A | 95.9 | P |
| | | gi = 3252980/ug = Mm 27860/len = 1749/STRA = for | | | | | | |
| 12 | 93112_at | Cluster Incl D86725 Mouse mRNA for mMCM2, complete cds/cds = (50,2764)/gb = D86725/gi = 2381484/ug = Mm 16711/ | 148.7 | A | 180 | A | 149.3 | P |
| | | len = 3290/STRA = for | | | | | | |
| 12 | 94829_at | Cluster Incl AW121838 UI-M-BH2 3-anx-h-10-0-UI s2 Mus musculus cDNA, 3 end/clone = UI-M-BH2 3-anx-h-10-0-UI/ | 219.7 | P | 274.7 | A | 283.4 | P |
| | | clone_end = 3/gb = AW121838/gi = 6097212/ug = Mm 22746/len = 319/STRA = for | | | | | | |
| 12 | 97470_at | Cluster Incl D86424 Mus musculus mRNA for high-sulfur keratin protein, partial cds/cds = (0,291)/gb = D86424/gi = 1944400/ | −257 | A | 754.9 | P | 780.7 | A |
| | | ug = Mm 30539/len = 409/STRA = for | | | | | | |
| 12 | 98973_at | Cluster Incl AA982595: uh10e10 r1 Mus musculus cDNA, 5 end/clone = IMAGE-1617546/clone_end = 5/gb = AA982595/ | 79.5 | A | 207.4 | P | 168.2 | A |
| | | gi = 3161254/ug = Mm 36583/len = 407/STRA = for | | | | | | |
| 12 | 100947_at | Cluster Incl AI847906: UI-M-AI1-afl-a-11-0-UI s1 Mus musculus cDNA, 3 end/clone = UI-M-AI1-afl-a-11-0-UI/clone_end = 3/ | 121.9 | M | 150.7 | A | 76.1 | P |
| | | gb = AI847906/gi = 5491812/ug = Mm 640/len = 510/STRA = for | | | | | | |
| 12 | 103957_at | Cluster Incl X57349 M musculus mRNA for transfemn receptor/cds = (0,2291)/gb = X57349/gi = 54914/ug = Mm 25560/ | 65.8 | P | 111.4 | P | 64 | A |
| | | len = 2292/STRA = for | | | | | | |
| 12 | 104427_at | Cluster Incl AA930526: vo45f08 r1 Mus musculus cDNA, 5 end/clone = IMAGE-1052871/clone_end = 5/gb = AA930526/ | 98.6 | A | 377.7 | A | 363.4 | P |
| | | gi = 3080016/ug = Mm 29172/len = 410/STRA = for | | | | | | |
| 12 | 104468_at | Cluster lncl AW045530: UI-M-BH1-aks-c-05-0-UI s1 Mus musculus cDNA, 3 end/clone = UI-M-BH1-aks-c-05-0-UI/ | 74.3 | A | 141.2 | P | 118.3 | M |
| | | clone_end = 3/gb = AW045530/gi = 5906059/ug = Mm 29755/len = 391/STRA = for | | | | | | |
| 12 | 104694_at | Cluster Incl AI152353: ud79d01.r1 Mus musculus cDNA, 5 end/clone = IMAGE-1477057/clone_end = 5/gb = AI152353/ | 59.2 | A | 194.8 | P | 137.5 | A |
| | | gi = 3680822/ug = Mm 3347/len = 386/STRA = for | | | | | | |
| 12 | 102102 _at | Cluster Incl C80410 C80410 Mus musculus cDNA, 3 end/clone = J0081D08/clone_end = 3/gb = C80410/gi = 2520740/ | 54.9 | A | 103.8 | P | 82.9 | A |
| | | ug = Mm 42447/len = 560/STRA = rev | | | | | | |
| 12 | 95655_at | Cluster Incl AA717740 vt98b12 r1 Mus musculus cDNA, 5 end/clone = IMAGE-1179187/clone_end = 5/gb = AA717740/ | 5.1 | A | 38.6 | P | 26.8 | A |
| | | gi = 2730014/ug = Mm 29540/len = 283/STRA = for | | | | | | |
| 12 | 99570_s_at | Cluster Incl AF029982 Mus musculus sarco(endo)plasmic reticulum calcium ATPase (SERCA2) gene, promoter region, | −137.1 | M | −0.9 | A | −37.5 | A |
| | | exons 1-3, and partial cds/cds = (523,741)/gb = AF029982/gi = 4103931/ug = Mm 42255/len = 742/STRA = for | | | | | | |
| 12 | 100565_at | Cluster Incl AW123396 UI-M-BH2.1-apf-h-12-0-UI s1 Mus musculus cDNA, 3 end/clone = UI-M-BH2.1-apf-h-12-0-UI/ | 37.5 | A | 126.5 | A | 97 | A |
| | | clone_end = 3/gb = AW123396/gi = 6098926/ug = Mm 5736/len = 420/STRA = for | | | | | | |
| 12 | 101515_at | Cluster Incl AF006688: Mus musculus peroxisomal acyl-CoA oxidase (muspaox) mRNA, complete cds/cds = (80,2065)/ | 73.6 | A | 184.4 | P | 136.7 | M |
| | | gb = AF006688/gi = 2253379/ug = Mm 8403/len = 3778/STRA = for | | | | | | |
| 12 | 102004_at | Cluster Incl AI530403 ui92f02.y1 Mus musculus cDNA, 5 end/clone = IMAGE-1889883/clone_end = 5/gb = AI530403/ | −198.7 | A | −116.5 | P | 33.4 | A |
| | | gi = 4444538/ug = Mm 99132/len = 906/STRA = for | | | | | | |
| 12 | 103033_at | Cluster Incl X06454 Mouse mRNA for sex-limited protein Slp(w7) alpha-gamma chain/cds = (0,1784)/gb = X06454/gi = 54105/ | 96 | A | 210.2 | A | 126.5 | M |
| | | ug = Mm 16106/len = 1893/STRA = for | | | | | | |
| 12 | 103427_at | Cluster Incl AW123223 UI-M-BH2.1-apg-e-07-0-UI s1 Mus musculus cDNA, 3 end/clone = UI-M-BH2 1-apg-e-07-0-UI/ | 173.1 | P | 216.2 | A | 205.8 | P |
| | | clone_end = 3/gb = AW123223/gi = 6098793/ug = Mm.18968/len = 410/STRA = for | | | | | | |
| 12 | 103658_r_at | Cluster Incl AI836552 UI-M-AP0-abe-e-08-0-UI.s1 Mus musculus cDNA, 3 end/clone = UI-M-AP0-abe-e-08-0-UI/ | 53.4 | A | 102.6 | A | 58.9 | A |
| | | clone_end = 3/gb = AI836552/gi = 5470765/ug = Mm.20559/len = 417/STRA = for | | | | | | |
| 12 | 100925_at | Cluster Incl AI131982 uc36a02 r1 Mus musculus cDNA, 5 end/clone = IMAGE-1400042/clone_end = 5/gb = AI131982/ | 39.1 | A | 89.3 | P | 23.6 | A |
| | | gi = 3601998/ug = Mm.608/len = 398/STRA = for | | | | | | |
| 12 | 103963_f_at | Cluster Incl AA914345: vy94a08 r1 Mus musculus cDNA, 5 end/clone = IMAGE-1313846/clone_end = 5/gb = AA914345/ | 22 | A | 86 | A | 56.2 | P |
| | | gi = 3053737/ug = Mm.25624/len = 452/STRA = for | | | | | | |
| 12 | 92338_f_at | Cluster Incl AI957030 ul74a01 x1 Mus musculus cDNA, 3 end/clone = IMAGE-2136264/clone_end = 3/gb = AI957030/ | −22.4 | A | 48.5 | P | 6.8 | A |
| | | gi = 5749739/ug = Mm 39024/len = 550/STRA = for | | | | | | |
| 12 | 100389_at | Cluster Incl AF021231 Mus musculus acetylcholinesterase-associated collagen (COIQ) gene/cds = (0,710)/gb = AF021231/ | 364.8 | P | 569.2 | P | 491.2 | A |
| | | gi = 2460054/ug = Mm 89475/len = 711/STRA = for | | | | | | |
| 13 | 92578_at | Cluster Incl U10118 Mus musculus endothelial-monocyte activating polypeptide II mRNA, complete cds/cds = (63,995)/ | 924 | P | 730.3 | P | 828.1 | P |
| | | gb = U10118/gi = 498911/ug = Mm.10507/len = 1068/STRA = for | | | | | | |
| 13 | 92633_at | Cluster Incl AJ242663 Mus musculus mRNA for cathepsin Z precursor (ctsZ gene)/cds = (104,1024)/gb = AJ242663/ | 390.1 | P | 199.5 | P | 210.1 | P |
| | | gi = 5019547/ug = Mm 115/len = 1291/STRA = for | | | | | | |
| 13 | 92794_f_at | Cluster Incl M35970 Mouse tumor metastatic process-associated protein (NM23) gene mRNA, 3 end/cds = (0,500)/ | 165.1 | P | 130.8 | P | 139.5 | P |
| | | gb = M35970/gi = 200066/ug = Mm 1260/len = 664/STRA = for | | | | | | |
| 13 | 92800_i_at | Cluster Incl AI836694: UI-M-AJ0-aax-a-08-0-UI s1 Mus musculus cDNA, 3 end/clone = UI-M-AJ0-aax-a-08-0-UI/ | 3511.6 | P | 2499.1 | P | 2683.7 | P |
| | | clone_end = 3/gb = AI836694/gi = 5470907/ug = Mm 12677/len = 344/STRA = for | | | | | | |
| 13 | 92804_at | Cluster Incl AW122864 UI-M-BH2 1-apa-a-02-0-UI s1 Mus musculus cDNA, 3 end/clone = UI-M-BH2 1-apa-a-02-0-UI/ | 397.7 | P | 326.4 | P | 417.6 | P |
| | | clone_end = 3/gb = AW122864/gi = 6098394/ug = Mm 12706/len = 346/STRA = for | | | | | | |
| 13 | 92807_at | Cluster Incl X77585: Thioredoxin/cds = (74,391)/gb = X77585/gi = 453971/ug = Mm 1275/len = 538/STRA = for | 3263 | P | 3383.6 | P | 3557.9 | P |
| 13 | 92834_at | Cluster Incl X51528: Tissue specific transplantation antigen P198-7/cds = (18,629)/gb = X51528/gi = 55049/ug = Mm.13020/ | 1082.6 | P | 699.5 | P | 1044.6 | P |
| | | len = 654/STRA = for | | | | | | |
| 13 | 92848_at | Cluster Incl X64837: M. musculus Oat mRNA for omithine aminotransferase/cds = (114,1433)/gb = X64837/gi = 53458/ | 742.8 | P | 583.1 | P | 670.1 | P |
| | | ug = Mm.13694/len = 2125/STRA = for | | | | | | |
| 13 | 92884_at | Cluster Incl AW045468: UI-M-BH1-akr-b-07-0-UI s1 Mus musculus cDNA, 3 end/clone = UI-M-BH1-akr-b-07-0-UI/ | 2587.6 | P | 2630.3 | P | 2778.5 | P |
| | | clone_end = 3/gb = AW045468/gi = 5905962/ug = Mm.14534/len = 318/STRA = for | | | | | | |
| 13 | 93008_at | Cluster Incl AW120557 UI-M-BH1-ana-f-03-0-UI s2 Mus musculus cDNA, 3 end/clone = UI-M-BH1-ana-f-03-0-UI/ | 788.3 | P | 483.8 | P | 690.7 | P |
| | | clone_end = 3/gb = AW120557/gi = 6095890/ug = Mm 14585/len = 530/STRA = for | | | | | | |
| 13 | 93019_at | Cluster Incl Z35401 M musculus C3H gene for histone H2A.X/cds = (0,431)/gb = Z35401/gi = 515878/ug = Mm 14767/ | 1828.4 | P | 930.1 | P | 972.4 | P |
| | | len = 432/STRA = for | | | | | | |
| 13 | 93254_at | Cluster Incl AI839142 UI-M-AO0-ach-d-11-0-UI s1 Mus musculus cDNA, 3 end/clone = UI-M-AO0-ach-d-11-0-UI/ | 1558.4 | P | 1278.3 | P | 1296.1 | P |
| | | clone_end = 3/gb = AI839142/gi = 5473355/ug = Mm 1700/len = 523/STRA = for | | | | | | |
| 13 | 93257_at | Cluster Incl AW048287 UI-M-BH1-aly-f-10-0-UI s1 Mus musculus cDNA, 3 end/clone = UI-M-BH1-aly-f-10-0-UI/ | 564.2 | P | 524.9 | P | 522.7 | P |
| | | clone_end = 3/gb = AW048287/gi = 5908816/ug = Mm 1706/len = 402/STRA = for | | | | | | |
| 13 | 93281_at | Cluster Incl AF049125: Mus musculus taipoxin-associated calcium binding protein 49 mRNA, complete cds/cds = (193,1158)/ | 342.1 | P | 304.7 | P | 305.5 | P |
| | | gb = AF049125/gi = 2935465/ug = Mm 1782/len = 1790/STRA = for | | | | | | |
| 13 | 93298_at | Cluster Incl U34883 3-phosphoadenosine 5-phosphosulfate synthase 1/cds = (33,1907)/gb = U34883/gi = 1109675/ | 563.9 | P | 602.1 | P | 576.4 | P |
| | | ug = Mm 18161/len = 2479/STRA = for | | | | | | |
| 13 | 93326_at | Cluster Incl D26483: Mouse mRNA for PE31/TALLA, complete cds/cds = (5,739)/gb = D26483/gi = 685220/ug = Mm 18590/ | 499.3 | P | 451.9 | P | 428.4 | P |
| | | len = 1720/STRA = for | | | | | | |
| 13 | 93531_at | Cluster Incl AI853855: UI-M-BH0-ajr-e-09-0-UI s1 Mus musculus cDNA, 3 end/clone = UI-M-BH0-ajr-e-09-0-UI/ | 605.9 | P | 790.9 | P | 734.1 | P |
| | | clone_end = 3/gb = AI853855/gi = 5497761/ug = Mm 19834/len = 540/STRA = for | | | | | | |
| 13 | 93536_at | Cluster Incl L22472: Bcl2-associated X protein/cds = (0,578)/gb = L22472/gi = 388191/ug = Mm 19904/len = 579/STRA = for | 340.3 | P | 302.3 | P | 321 | P |
| 13 | 93548_at | Cluster Incl AW122942: UI-M-BH2.1-aoz-a-11-0-UI s2 Mus musculus cDNA, 3 end/clone = UI-M-BH2 1-aoz-a-11-0-UI/ | 813.2 | P | 681.9 | P | 895.5 | P |
| | | clone_end = 3/gb = AW122942/gi = 6098472/ug = Mm 20188/len = 540/STRA = for | | | | | | |
| 13 | 93551_at | Cluster Incl AW121424: UI-M-BH2.2-aon-b-07-0-UI s1 Mus musculus cDNA, 3 end/clone = UI-M-BH2 2-aon-b-07-0-UI/ | 1587.9 | P | 1602.1 | P | 1519.9 | P |
| | | clone_end = 3/gb = AW121424/gi = 6096757/ug = Mm 20207/len = 418/STRA = for | | | | | | |
| 13 | 93559_at | Cluster Incl D90374: Mouse mRNA for APEX nuclease, complete cds/cds = (66,1019)/gb = D90374/gi = 220336/ug = Mm 203/ | 751 | P | 632.7 | P | 789.8 | P |
| | | len = 1185/STRA = for | | | | | | |
| 13 | 93564_at | Cluster Incl AW122542: UI-M-BH2 2-aox-c-12-0-UI s1 Mus musculus cDNA, 3 end/clone = UI-M-BH2 2-aox-c-12-0-UI/ | 695.5 | P | 628.9 | P | 693.7 | P |
| | | clone_end = 3/gb = AW122542/gi = 6098066/ug = Mm 20353/len = 471/STRA = for | | | | | | |
| 13 | 93595_at | Cluster Incl AF111172: Mus musculus lysosomal pepstatin insensitive protease precursor (Cln2) mRNA, complete cds/ | 825.7 | P | 696.1 | P | 870.3 | P |
| | | cds = (28,1716)/gb = AF111172/gi = 4106912/ug = Mm 20837/len = 2270/STRA = for | | | | | | |
| 13 | 93599_at | Cluster Incl AI841260: UI-M-AM0-adu-d-05-0-UI s1 Mus musculus cDNA, 3 end/clone = UI-M-AM0-adu-d-05-0-UI/ | 1148 | P | 816.1 | P | 768.8 | P |
| | | clone_end = 3/gb = AI841260/gi = 5475478/ug = Mm 20877/len = 510/STRA = for | | | | | | |
| 13 | 93815_at | Cluster Incl AI839425: UI-M-AN0-aco-e-06-0-UI s1 Mus musculus cDNA, 3 end/clone = UI-M-AN0-aco-e-06-0-UI/ | 1228.1 | P | 1297.8 | P | 1112.9 | P |
| | | clone_end = 3/gb = AI839425/gi = 5473638/uq = Mm 21501/len = 402/STRA = for | | | | | | |
| 13 | 93833_s_at | Cluster Incl X05862: Mouse H2B and H2A histone genes (291A)/cds = (0,380)/gb = X05862/gi = 51302/ug = Mm 21579/ | 462.1 | P | 353.9 | P | 390.8 | P |
| | | len = 381/STRA = for | | | | | | |
| 13 | 93982_at | Cluster Incl AW122476: UI-M-BH2.2-aou-e-01-0-UI s1 Mus musculus cDNA, 3 end/clone = UI-M-BH2 2-aou-e-01-0-UI/ | 581.1 | P | 489.6 | P | 635.5 | P |
| | | clone_end = 3/gb = AW122476/gi = 6097997/ug = Mm 21707/len = 182/STRA = for | | | | | | |
| 13 | 94005_at | Cluster Incl AI157548: ue55h05 r1 Mus musculus cDNA, 5 end/clone = IMAGE-1495065/clone_end = 5/gb = AI157548/ | 268.7 | P | 228 | P | 292.1 | P |
| | | gi = 3686017/ug = Mm 21780/len = 625/STRA = for | | | | | | |
| 13 | 94017_s_at | Cluster Incl X98511 M musculus PR264 gene/cds = (0,365)/gb = X98511/gi = 1405746/ug = Mm 21841/len = 366/STRA = for | 4042.6 | P | 3451.3 | P | 3690 | P |
| 13 | 94040_at | Cluster Incl D73368: House mouse; musculus domesticus adult thymus mRNA for Mer protein, complete cds/cds = (41,355)/ | 2377 | P | 1820.7 | P | 2183.3 | P |
| | | gb = D73368/gi = 1620873/ug = Mm 21952/len = 754/STRA = for | | | | | | |
| 13 | 94076_i_at | Cluster Incl D31717 Mouse MARib mRNA for ribophorin, complete cds/cds = (41,1864)/gb = D31717/gi = 1468960/ | 808.2 | P | 607.9 | P | 606.5 | P |
| | | ug = Mm 22130/len = 2192/STRA = for | | | | | | |
| 13 | 94077_f_at | Cluster Incl D31717 Mouse MARib mRNA for ribophorin, complete cds/cds = (41,1864)/gb = D31717/gi = 1468960/ | 1491.7 | P | 1071.2 | P | 1259.6 | P |
| | | ug = Mm 22130/len = 2192/STRA = for | | | | | | |
| 13 | 94209_g_at | Cluster Incl AW045202 UI-M-BH1-alh-08-0-UI.s1 Mus musculus cDNA, 3 end/clone = UI-M-BH1-alh-c-08-0-UI/ | 496.8 | P | 455.4 | P | 411.7 | P |
| | | clone_end = 3/gb = AW045202/gi = 5905731/ug = Mm 2220/len = 430/STRA = for | | | | | | |
| 13 | 94259_at | Cluster Incl AB024935 Mus musculus Sid3177 mRNA, complete cds/cds = (111,593)/gb = AB024935/gi = 5931546/ | 811.2 | P | 797.2 | P | 702 | P |
| | | ug = Mm 22421/len = 1510/STRA = for | | | | | | |
| 13 | 94273_at | Cluster Incl AI849067: UI-M-AH1-agw-d-04-0-UI s1 Mus musculus cDNA, 3 end/clone = UI-M-AH1-agw-d-04-0-UI/ | 605.6 | P | 536.5 | P | 534 | P |
| | | clone_end = 3/gb = AI849067/gi = 5492973/ug = Mm 22485/len = 507/STRA = for | | | | | | |
| 13 | 94292_at | Cluster Incl AF096285 Mus musculus serine-threonine kinase receptor-associated protein mRNA, complete cds/ | 606.4 | P | 648.2 | P | 656.2 | P |
| | | cds = (85,1140)/gb = AF096285/gi = 4063382/ug = Mm.22584/len = 1630/STRA = for | | | | | | |
| 13 | 94323_at | Cluster Incl AW048132 UI-M-BH1-alq-f-07-0-UI s1 Mus musculus cDNA, 3 end/clone = UI-M-BH1-alq-f-07-0-UI/ | 1595.9 | P | 1493.6 | P | 1478.7 | P |
| | | clone_end = 3/gb = AW048132/gi = 5908661/ug = Mm 22667/len = 271/STRA = for | | | | | | |
| 13 | 95404_at | Cluster Incl AW123453 UI-M-BH2 1-apj-e-11-0-UI.s1 Mus musculus cDNA, 3 end/clone = UI-M-BH2 1-apj-e-11-0-UI/ | 381.9 | P | 275.7 | P | 288.7 | P |
| | | clone_end = 3/gb = AW123453/gi = 6098983/ug = Mm 24199/len = 274/STRA = for | | | | | | |
| 13 | 95445_at | Cluster Incl AW124194 UI-M-BH2 1-aph-b-0I-0-UI s1 Mus musculus cDNA, 3 end/clone = UI-M-BH2.1-aph-b-01-0-UI/ | 300.7 | P | 362.5 | P | 401.3 | P |
| | | clone_end = 3/gb = AW124194/gi = 6099724/ug = Mm 24593/len = 341/STRA = for | | | | | | |
| 13 | 95473_s_at | Cluster Incl AW123338 UI-M-BH2 1-apf-c-12-0-UI s1 Mus musculus cDNA, 3 end/clone = UI-M-BH2 1-apl-c-12-0-UI/ | 884.9 | P | 830.1 | P | 849.8 | P |
| | | clone_end = 3/gb = AW123338/gi = 6098868/ug = Mm.24808/len = 425/STRA = for | | | | | | |
| 13 | 95491_at | Cluster Incl AB015652 Mus musculus gene for DJ-1, complete cds/cds = (0,521)/gb = AB015652/gi = 3256342/ug = Mm 2507/ | 1154.9 | P | 712.8 | P | 844.5 | P |
| | | len = 522/STRA = for | | | | | | |
| 13 | 96016_at | Cluster Incl AW045665 UI-M-BH1-alg-h-11-0-UI.s1 Mus musculus cDNA, 3 end/clone = UI-M-BH1-alg-h-11-0-UI/ | 727.5 | P | 310.6 | P | 413.3 | P |
| | | clone_end = 3/gb = AW045665/gi = 5906194/ug = Mm.25590/len = 395/STRA = for | | | | | | |
| 13 | 96024_at | Cluster Incl L32838: Mus musculus (clone C7/B9) S-adenosyl homocysteine hydrolase (ahcy) mRNA, complete cds/ | 774.3 | P | 602.3 | P | 791.4 | P |
| | | cds = (5,1303)/gb = L32836/gi = 904131/ug = Mm.2573/len = 2057/STRA = for | | | | | | |
| 13 | 96025_g_at | Cluster Incl L32836: Mus musculus (clone C7/B9) S-adenosyl homocysteine hydrolase (ahcy) mRNA, complete cds/ | 1104.3 | P | 715.7 | P | 1151.2 | P |
| | | cds = (5,1303)/gb = L32836/gi = 904131/ug = Mm.2573/len = 2057/STRA = for | | | | | | |
| 13 | 96632_at | Cluster Incl AB025049: Mus musculus mRNA for Sid393p, complete cds/cds = (96,962)/gb = AB025049/gi = 5931552/ | 2522 | P | 2028.8 | P | 2072.5 | P |
| | | ug = Mm.27218/len = 1621/STRA = for | | | | | | |
| 13 | 96635_at | Cluster Incl AW125392 UI-M-BH2 3-aqh-c-08-0-UI s1 Mus musculus cDNA, 3 end/clone = UI-M-BH2 3-aqh-c-08-0-UI/ | 199.1 | P | 204.2 | P | 154.6 | P |
| | | clone_end = 3/gb = AW125392/gi = 6100922/ug = Mm 27235/len = 463/STRA = for | | | | | | |
| 13 | 96696_at | Cluster Incl AI837110 UI-M-AK0-adc-e-02-0-UI s1 Mus musculus cDNA, 3 end/clone = UI-M-AK0-adc-e-02-0-UI/ | 1564.1 | P | 1122.4 | P | 1385.8 | P |
| | | clone_end = 3/gb = AI837110/gi = 5471323/ug = Mm.27545/len = 124/STRA = for | | | | | | |
| 13 | 97247_at | Cluster Incl AW124778 UI-M-BH2.1-aqa-g-10-0-UI s1 Mus musculus cDNA, 3 end/clone = UI-M-BH2 1-aqa-g-10-0-UI/ | 730.4 | P | 654.5 | P | 638.7 | P |
| | | clone_end = 3/gb = AW124778/gi = 6100308/ug = Mm 27847/len = 462/STRA = for | | | | | | |
| 13 | 97250_at | Cluster Incl AW049326: UI-M-BH1-amr-f-01-0-UI s1 Mus musculus cDNA, 3 end/clone = UI-M-BH1-amr-f-01-0-UI/ | 1074.9 | P | 1135.7 | P | 1040.4 | P |
| | | clone_end = 3/gb = AW049326/gi = 5909855/ug = Mm 27858/len = 234/STRA = for | | | | | | |
| 13 | 97309_at | Cluster Incl AW214164 uo41a02.x1 Mus musculus cDNA, 3 end/clone = IMAGE-2645066/clone_end = 3/gb = AW214164/ | 397.4 | P | 432.9 | P | 556 | P |
| | | gi = 6520606/ug = Mm 28066/len = 253/STRA = for | | | | | | |
| 13 | 97311_at | Cluster Incl AIB36509: UI-M-AI0-aal-f-06-0-UI.s1 Mus musculus cDNA, 3 end/clone = UI-M-AI0-aal-f-06-0-UI/clone_end = 3/ | 1502.7 | P | 1327.3 | P | 1533.4 | P |
| | | gb = AI836509/gi = 5470722/ug = Mm 28077/len = 320/STRA = for | | | | | | |
| 13 | 97809_at | Cluster Incl AF109906 Mus musculus MHC class III region RD gene, partial cds, Bf, C2, G9A, NG22, G9, HSP70, HSP70, | 920.2 | P | 846.8 | P | 986.7 | P |
| | | HSC70t, and smRNP genes, complete cds, G7A gene, partial cds, and unknown genes/cds = (0,3002)/gb = AF109906/ | | | | | | |
| | | gi = 3986763/ug = Mm 28155/len = 300/STRA = for | | | | | | |
| 13 | 97824_at | Cluster Incl AW121031 UI-M-BH2 3-aof-b-04-0-UI s1 Mus musculus cDNA, 3 end/clone = UI-M-BH2 3-aof-b-04-0-UI/ | 1503.6 | P | 1103.5 | P | 1814.4 | P |
| | | clone_end = 3/gb = AW121031/gi = 6096364/ug = Mm 28203/len = 396/STRA = for | | | | | | |
| 13 | 97836_at | Cluster Incl AI843444 UI-M-AQ1-aec-d-07-0-UI.s1 Mus musculus cDNA, 3 end/clone = UI-M-AQ1-aec-d-07-0-UI/ | 936.7 | P | 809.7 | P | 765.1 | P |
| | | clone_end = 3/gb = AI843444/gi = 5477657/ug = Mm 28235/len = 315/STRA = for | | | | | | |
| 13 | 97907_at | Cluster Incl AW049564 UI-M-BH1-anf-g-07-0-UI s1 Mus musculus cDNA, 3 end/clone = UI-M-BH1-anf-g-07-0-UI/ | 507.8 | P | 342.8 | P | 480.2 | P |
| | | clone_end = 3/gb = AW049564/gi = 5910093/ug = Mm 28470/len = 426/STRA = for | | | | | | |
| 13 | 98904_at | Cluster Incl AW061339 UI-M-BH1-anw-g-01-0-UI s1 Mus musculus cDNA, 3 end/clone = UI-M-BH1-anw-g-01-0-UI/ | 410.7 | P | 325.2 | P | 354.7 | P |
| | | clone_end = 3/gb = AW061339/gi = 6009090/ug = Mm 28579/len = 376/STRA = for | | | | | | |
| 13 | 98928_at | Cluster Incl AW122820 UI-M-BH2 1-aoz-b-12-0-UI s1 Mus musculus cDNA, 3 end/clone = UI-M-BH2 1-aoz-b-12-0-UI/ | 448.9 | P | 359 | P | 363.8 | P |
| | | clone_end = 3/gb = AW122820/gi = 6098350/ug = Mm 28652/len = 302/STRA = for | | | | | | |
| 13 | 98930_at | Cluster Incl AI844701: UI-M-AL1-ahr-h-03-0-UI s1 Mus musculus cDNA, 3 end/clone = UI-M-AL1-ahr-h-03-0-UI/ | 490.4 | P | 392.6 | P | 793.8 | P |
| | | clone_end = 3/gb = AI844701/gi = 5488607/ug = Mm 28668/len = 487/STRA = for | | | | | | |
| 13 | 94455_at | Cluster Incl AA795062 vs09d03 r1 Mus musculus cDNA, 5 end/clone = IMAGE-1137701/clone_end = 5/gb = AA795062/ | 707.3 | P | 449.8 | P | 547.5 | P |
| | | gi = 2858017/ug = Mm 28803/len = 536/STRA = for | | | | | | |
| 13 | 94503_at | Cluster Incl AI842492 UI-M-AM1-afy-h-05-0-UI s1 Mus musculus cDNA, 3 end/clone = UI-M-AM1-afy-h-05-0-UI/ | 674.5 | P | 598.8 | P | 548.4 | P |
| | | clone_end = 3/gb = AI842492/gi = 5476705/ug = Mm 28952/len = 259/STRA = for | | | | | | |
| 13 | 94510_at | Cluster Incl AI853476 UI-M-BH0-ajy-h-09-0-UI s1 Mus musculus cDNA, 3 end/clone = UI-M-BH0-ajy-h-09-0-UI/ | 450.4 | P | 537.7 | P | 506.4 | P |
| | | clone_end = 3/gb = AI853476/gi = 5497382/ug = Mm 28975/len = 537/STRA = for | | | | | | |
| 13 | 94524_at | Cluster Incl AW123469 UI-M-BH2 1-apj-g-04-0-UI.s1 Mus musculus cDNA, 3 end/clone = UI-M-BH2.1-apj-g-04-0-UI/ | 333.7 | P | 233.7 | P | 309.5 | P |
| | | clone_end = 3/gb = AW123469/gi = 6098999/ug = Mm 29028/len = 452/STRA = for | | | | | | |
| 13 | 94532_at | Cluster Incl U13841.ATPase, H+ transporting lysosomal (vacuolar proton pump) 32 kDa/cds = (0,686)/gb = U13841/ | 892.3 | P | 822.5 | P | 849.6 | P |
| | | gi = 1184662/ug = Mm.29045/len = 687/STRA = for | | | | | | |
| 13 | 95049_at | Cluster Incl AI837853 UI-M-AP0-abk-d-04-0-UI s1 Mus musculus cDNA, 3 end/clone = UI-M-AP0-abk-d-04-0-UI/ | 2784 | P | 2036.4 | P | 2668.5 | P |
| | | clone_end = 3/gb = AI837853/gi = 5472066/ug = Mm 29135/len = 429/STRA = for | | | | | | |
| 13 | 95067_at | Cluster Incl AI837821 UI-M-AK0-adl-h-06-0-UI s1 Mus musculus cDNA, 3 and/clone = UI-M-AK0-adl-h-06-0-UI/ | 303.1 | P | 105 | P | 250.7 | P |
| | | clone_end = 3/gb = AI837821/gi = 5472034/ug = Mm 29183/len = 468/STRA = for | | | | | | |
| 13 | 95734_at | Cluster Incl AI852230 UI-M-BH0-ajc-f-07-0-UI s1 Mus musculus cDNA, 3 end/clone = UI-M-BH0-ajc-f-07-0-UI/clone_end = 3/ | 651.7 | P | 519.9 | P | 604.7 | P |
| | | gb = AI852230/gi = 5496136/ug = Mm 29746/len = 392/STRA = for | | | | | | |
| 13 | 95743_at | Cluster Incl AI846078 UI-M-AN1-afj-a-03-0-UI s2 Mus musculus cDNA, 3 end/clone = UI-M-AN1-afj-a-03-0-UI/ | 1911.4 | P | 1267.7 | P | 1282.2 | P |
| | | clone_end = 3/gb = AI846078/gi = 5490024/ug = Mm.29769/len = 335/STRA = for | | | | | | |
| 13 | 96242_at | Cluster Incl AW125751 UI-M-BH2 2-aqi-h-05-0-UI s1 Mus musculus cDNA, 3 end/clone = UI-M-BH2.2-aqi-h-05-0-UI/ | 396.7 | P | 284.8 | P | 321.1 | P |
| | | clone_end = 3/gb = AW125751/gi = 6101246/ug = Mm 29802/len = 240/STRA = for | | | | | | |
| 13 | 96261 _at | Cluster Incl AW122694 UI-M-BH2.2-aot-b-04-0-UI s1 Mus musculus cDNA, 3 end/clone = UI-M-BH2 2-aot-b-04-0-UI/ | 1163.6 | P | 982.4 | P | 964.5 | P |
| | | clone_end = 3/gb = AW122694/gi = 6098224/ug = Mm 29828/len = 456/STRA = for | | | | | | |
| 13 | 96280_at | Cluster Incl AI851603 UI-M-BH0-aln-a-07-0-UI.s1 Mus musculus cDNA, 3 end/clone = UI-M-BH0-aln-a-07-0-UI/ | 1131.1 | P | 896.5 | P | 894 | P |
| | | clone_end = 3/gb = AI851603/gi = 5495509/ug = Mm.29867/len = 415/STRA = for | | | | | | |
| 13 | 96295_at | Cluster Incl AW122030 UI-M-BH2 2-aoo-b-05-0-UI.s1 Mus musculus cDNA, 3 end/clone = UI-M-BH2.2-aoo-b-05-0-UI/ | 1578.5 | P | 1436.3 | P | 2191.9 | P |
| | | clone_end = 3/gb = AW122030/gi = 6097436/ug = Mm 29902/len = 358/STRA = for | | | | | | |
| 13 | 96318_at | Cluster Incl AW045739-UI-M-BH1-akq-g-08-0-UI s1 Mus musculus cDNA, 3 end/clone = UI-M-BH1-akq-g-08-0-UI/ | 483 | P | 375.5 | P | 472.5 | P |
| | | clone_end = 3/gb = AW045739/gi = 5906268/ug = Mm 29925/len = 263/STRA = for | | | | | | |
| 13 | 96352_at | Cluster Incl AI848845 UI-M-AJ1-ahb-d-02-0-UI.s1 Mus musculus cDNA, 3 end/clone = UI-M-AJ1-ahb-d-02-0-UI/ | 941.2 | P | 647.2 | P | 659.1 | P |
| | | clone_end = 3/gb = AI848845/gi = 5492751/ug = Mm 30003/len = 402/STRA = for | | | | | | |
| 13 | 96847_at | Cluster Incl AI845393 UI-M-AO1-aej-a-03-0-UI s1 Mus musculus cDNA, 3 end/clone = UI-M-AO1-aoj-a-03-0-UI/ | 972.9 | P | 706.2 | P | 630.2 | P |
| | | clone_end = 3/gb = AI845393/gi = 5489299/ug = Mm 30028/len = 278/STRA = for | | | | | | |
| 13 | 96864_at | Cluster Incl AI848770: UI-M-AJ1-ahg-a-08-0-UI s1 Mus musculus cDNA, 3 end/clone = UI-M-AJ1-ahg-a-08-0-UI/ | 358.7 | P | 353.8 | P | 308 | P |
| | | clone_end = 3/gb = AI848770/gi = 5492676/ug = Mm 30057/len = 383/STRA = for | | | | | | |
| 13 | 96959_at | Cluster Incl AW210080: ul50f12.y1 Mus musculus cDNA, 5 end/clone = IMAGE-2101775/clone_end = 5/gb = AW210080/ | 1391.9 | P | 1551.7 | P | 1434.6 | P |
| | | gi = 6516020/ug = Mm 30233/len = 505/STRA = for | | | | | | |
| 13 | 97458_at | Cluster Incl AI845935: UI-M-AK1-aex-f-04-0-UI s1 Mus musculus cDNA, 3 end/clone = UI-M-AK1-aex-f-04-0-UI/ | 1189 | P | 1088.6 | P | 985 | P |
| | | clone_end = 3/gb = AI845935/gi = 5489841/ug = Mm 30267/len = 380/STRA = for | | | | | | |
| 13 | 97472_at | Cluster Incl AJ006341 Mus musculus mRNA for peroxisomal integral membrane protein PMP34/cds = (47,970)/ | 675.9 | P | 652.8 | P | 715.6 | P |
| | | gb = AJ006341/gi = 3183980/ug = Mm 306/1en = 1700/STRA = for | | | | | | |
| 13 | 97477_at | Cluster Incl AW124594: UI-M-BH2.1-apt-g-02-0-UI.s1 Mus musculus cDNA, 3 end/clone = UI-M-BH2.1-apt-g-02-0-UI/ | 357.3 | P | 407.4 | P | 394.2 | P |
| | | clone_end = 3/gb = AW124594/gi = 6100124/ug = Mm.30718/len = 407/STRA = for | | | | | | |
| 13 | 97479_at | Cluster Incl AJ130961: Mus musculus mRNA for ubiquitin-conjugating enzyme/cds = (20,484)/gb = AJ130961/gi = 3928492/ | 1064.7 | P | 990.6 | P | 1174.3 | P |
| | | ug = Mm 3074/len = 2597/STRA = for | | | | | | |
| 13 | 97535_at | Cluster Incl D87661: Mus musculus mRNA for 14-3-3 eta, complete cds/cds = (184,924)/gb = D87661/gi = 1526540/ | 743.3 | P | 511.3 | P | 636.5 | P |
| | | ug = Mm 3308/len = 1098/STRA = for | | | | | | |
| 13 | 98037_at | Cluster Incl AW122782 UI-M-BH2 1-aoy-d-05-0-UI s1 Mus musculus cDNA, 3 end/clone = UI-M-BH2 1-aoy-d-05-0-UI/ | 490.2 | P | 399.3 | P | 496.8 | P |
| | | clone_end = 3/gb = AW122782/gi = 6098312/ug = Mm 33907/len = 153/STRA = for | | | | | | |
| 13 | 98107_at | Cluster Incl AW123801 UI-M-BH2 1-apm-e-08-0-UI s1 Mus musculus cDNA, 3 end/clone = UI-M-BH2 1-apm-e-08-0-UI/ | 1321.6 | P | 1195.3 | P | 1282 | P |
| | | clone_end = 3/gb = AW123801/gi = 6099331/ug = Mm.34796/len = 367/STRA = for | | | | | | |
| 13 | 98129_at | Cluster Incl AI852553 UI-M-BH0-aiw-g-02-0-UI s1 Mus musculus cDNA, 3 end/clone = UI-M-BH0-aiw-g-02-0-UI/ | 3970.2 | P | 2865.7 | P | 3347.9 | P |
| | | clone_end = 3/gb = AI852553/gi = 5496459/ug = Mm 3532/len = 276/STRA = for | | | | | | |
| 13 | 98533_at | Cluster Incl AI854779 UI-M-BH0-aka-d-08-0-UI s1 Mus musculus cDNA, 3 end/clone = UI-M-BH0-aka-d-08-0-UI/ | 1422 | P | 972.8 | P | 923.3 | P |
| | | clone_end = 3/gb = AI854779/gi = 5498685/ug = Mm 35849/len = 428/STRA = for | | | | | | |
| 13 | 98574_at | Cluster Incl AI835685 UI-M-AJ0-aau-g-09-0-UI s1 Mus musculus cDNA, 3 end/clone = UI-M-AJ0-aau-g-09-0-UI/ | 619 | P | 668.7 | P | 612.8 | P |
| | | clone_end = 3/gb = AI835685/gi = 5469898/ug = Mm 3757/len = 227/STRA = for | | | | | | |
| 13 | 98609_at | Cluster Incl AJ250723 Mus musculus mRNA for septin-like protein Sint1 (Sint1 gene)/cds = (177,1181)/gb = AJ250723/ | 1696.4 | P | 1483.2 | P | 1661.9 | P |
| | | gi = 6165418/ug = Mm 38450/len = 2942/STRA = for | | | | | | |
| 13 | 99106_at | Cluster Incl AF071315 Mus musculus COP9 complex subunit 6 (COPS6) mRNA, complete cds/cds = (48,1022)/ | 1278.2 | P | 906.5 | P | 1176.4 | P |
| | | gb = AF071315/gi = 3309171/ug = Mm 3981/len = 1160/STRA = for | | | | | | |
| 13 | 99113_at | Cluster Incl AF071313 Mus musculus COP9 complex subunit 3 (COPS3) mRNA, complete cds/cds = (43,1314)/ | 499.6 | P | 477.5 | P | 510.1 | P |
| | | gb = AF071313/gi = 3309167/ug = Mm 40/len = 1583/STRA = for | | | | | | |
| 13 | 99593_at | Cluster Incl AI852355: UI-M-BH0-ajo-c-05-0-UI s1 Mus musculus cDNA, 3 end/clone = UI-M-BH0-ajo-c-05-0-UI/ | 1569.8 | P | 871.9 | P | 1240.7 | P |
| | | clone_end = 3/gb = AI852355/gi = 5496261/ug = Mm 42805/len = 413/STRA = for | | | | | | |
| 13 | 99596_f_at | Cluster Incl M13963 Guanine nucleotide binding protein, alpha inhibiting 2/cds = (97,1164)/gb = M13963/gi = 193513/ | 5988.7 | P | 4804.1 | P | 5446.6 | P |
| | | ug = Mm 4284/len = 1316/STRA = for | | | | | | |
| 13 | 99598_g_at | Cluster Incl AI841629 UI-M-AO0-acg-c-01-0-UI s1 Mus musculus cDNA, 3 end/clone = UI-M-AO0-acg-c-01-0-UI/ | 2534.3 | P | 1987.6 | P | 2237.4 | P |
| | | clone_end = 3/gb = AI841629/gi = 5475842/ug = Mm 4284/len = 436/STRA = for | | | | | | |
| 13 | 99618_at | Cluster Incl AI853523: UI-M-BH0-ajd-e-03-0-UI s1 Mus musculus cDNA, 3 end/clone = UI-M-BH0-ajd-e-03-0-UI/ | 1140.5 | P | 678.7 | P | 1145.7 | P |
| | | clone_end = 3/gb = AI853523/gi = 5497429/ug = Mm 43162/len = 425/STRA = for | | | | | | |
| 13 | 99619_at | Cluster Incl AIB47483 UI-M-AI1-afs-g-03-0-UI s1 Mus musculus cDNA, 3 end/clone = UI-M-AI1-afs-g-03-0-UI/clone_end = 3/ | 144.5 | P | 66.2 | P | 144.3 | P |
| | | gb = AI847483/gi = 5491389/ug = Mm 43212/len = 471/STRA = for | | | | | | |
| 13 | 99637_at | Cluster Incl AF011450 Procollagen, type XV/cds = (158,4259)/gb = AF011450/gi = 2558824/ug = Mm 4352/len = 5176/ | 771.8 | P | 650.5 | P | 693.2 | P |
| | | STRA = for | | | | | | |
| 13 | 99655_at | Cluster Incl AB025405 Mus musculus mRNA for sid2895p, complete cds/cds = (98,637)/gb = AB025405/gi = 5931558/ | 929.5 | P | 560.2 | P | 768.6 | P |
| | | ug = Mm 43760/len = 1113/STRA = for | | | | | | |
| 13 | 100040_at | Cluster Incl AI843081: UI-M-AK1-aes-b-06-0-UI s1 Mus musculus cDNA, 3 end/clone = UI-M-AK1-aes-b-06-0-UI/ | 231.3 | P | 104.7 | P | 136.8 | P |
| | | clone_end = 3/gb = AI843081/gi = 5477294/ug = Mm 44225/len = 232/STRA = for | | | | | | |
| 13 | 100050_at | Cluster Incl M31885 Inhibitor of DNA binding 1/cds = (0,532)/gb = M31885/gi = 194090/ug = Mm 444/len = 927/STRA = for | 1364.4 | P | 923.6 | P | 1070.7 | P |
| 13 | 100062_at | Cluster Incl X62154.Mini chromosome maintenance deficient (S cerevisiae)/cds = (0,2387)/gb = X62154/gi = 53550/ | 285.9 | P | 328.9 | P | 304.5 | P |
| | | ug = Mm 4502/len = 2813/STRA = rev | | | | | | |
| 13 | 100065_r_at | Cluster Incl M63801 Mus musculus connexin 43 (alpha-1 gap junction) mRNA, complete cds/cds = (220,1368)/gb = M63801/ | 2649.2 | P | 2505.5 | P | 1991.8 | P |
| | | gi = 191773/ug = Mm 4504/len = 1408/STRA = for | | | | | | |
| 13 | 100578_at | Cluster Incl M33934 Mouse IMP dehydrogenase mRNA, complete cds/cds = (66,1610)/gb = M33934/gi = 198393/ | 1226.9 | P | 1116.4 | P | 1089.3 | P |
| | | ug = Mm 6065/len = 1707/STRA = for | | | | | | |
| 13 | 100628_at | Cluster Incl AI840263 UI-M-AH0-act-e-04-0-UI.s1 Mus musculus cDNA, 3 end/clone = UI-M-AH0-act-e-04-0-UI/ | 987.8 | P | 562.1 | P | 825.4 | P |
| | | clone_end = 3/gb = AI840263/gi = 5474476/ug = Mm 666/len = 248/STRA = for | | | | | | |
| 13 | 101017_at | Cluster Incl AA791962: vs65h09 r1 Mus musculus cDNA, 5 end/clone = IMAGE-1151201/clone_end = 5/gb = AA791962/ | 578.3 | P | 471.8 | P | 493.5 | P |
| | | gi = 2854917/ug = Mm 6839/len = 336/STRA = rev | | | | | | |
| 13 | 101061_at | Cluster Incl AI845293 UI-M-BG0-ahw-f-04-0-UI.s1 Mus musculus cDNA, 3 end/clone = UI-M-BG0-ahw-f-04-0-UI/ | 1417.9 | P | 1110 | P | 1371 | P |
| | | clone_end = 3/gb = AI845293/gi = 5489199/ug = Mm 7091/len = 254/STRA = for | | | | | | |
| 13 | 101081_at | Cluster Incl AJ010483 Mus musculus mRNA for CtBP1 protein/cds = (99,1421)/gb = AJ010483/gi = 3452506/ug = Mm 7286/ | 1320.2 | P | 1288.5 | P | 1367.1 | P |
| | | len = 2240/STRA = for | | | | | | |
| 13 | 101104_at | Cluster Incl AB001990: Mus musculus mRNA for Dcra, complete cds/cds = (50,943)/gb = AB001990/gi = 2588992/ | 572.4 | P | 341.7 | P | 598.7 | P |
| | | ug = Mm 7472/len = 2157/STRA = for | | | | | | |
| 13 | 101105_at | Cluster Incl AB025349: Mus musculus L2BP1/BAF mRNA for LAP2 binding protein 1, complete cds/cds = (57,326)/ | 1604.2 | P | 1064.6 | P | 1172 | P |
| | | gb = AB025349/gi = 5708070/ug = Mm 7508/len = 603/STRA = for | | | | | | |
| 13 | 101519_at | Cluster Incl M29264 Mouse signal recognition particle subunit (SRP14) mRNA, complete cds/cds = (7,339)/gb = M29264/ | 1100.6 | P | 914 | P | 1018.1 | P |
| | | gi = 201062/ug = Mm.850/len = 706/STRA = for | | | | | | |
| 13 | 101521_at | Cluster Incl AB013819 Mus musculus mRNA for TIAP, complete cds/cds = (108,530)/gb = AB013819/gi = 3135206/ | 561.6 | P | 235 | P | 256.1 | P |
| | | ug = Mm.8552/len = 955/STRA = for | | | | | | |
| 13 | 101964_at | Cluster Incl U05809: Transketolase/cds = (91,1962)/gb = U05809/gi = 452485/ug = Mm.9307/len = 2062/STRA = for | 1041 | P | 712.4 | P | 1033.8 | P |
| 13 | 103364_f_at | Cluster Incl AW045256 UI-M-BH1-akn-a-02-0-UI.s1 Mus musculus cDNA, 3 end/clone = UI-M-BH1-akn-a-02-0-UI/ | 311.3 | P | 170.1 | P | 305 | P |
| | | clone_end = 3/gb = AW045256/gi = 5905785/ug = Mm.18520/len = 324/STRA = for | | | | | | |
| 13 | 103381_at | Cluster Incl AI838735 UI-M-AO0-acf-c-11-0-UI s1 Mus musculus cDNA, 3 end/clone = UI-M-AO0-acf-c-11-0-UI/ | 762.7 | P | 758.9 | P | 822.8 | P |
| | | clone_end = 3/gb = AI838735/gi = 5472948/ug = Mm.18634/len = 413/STRA = for | | | | | | |
| 13 | 103606_r_at | Cluster Incl AW121438 UI-M-BH2 2-aon-c-10-0-UI.s1 Mus musculus cDNA, 3 end/clone = UI-M-BH2 2-aon-c-10-0-UI/ | 370.2 | P | 268.1 | P | 350.2 | P |
| | | clone_end = 3/gb = AW121438/gi = 6096771/ug = Mm.20156/len = 269/STRA = for | | | | | | |
| 13 | 103620_a_at | Cluster Incl U77714: Survival motor neuron/cds = (0,866)/gb = U77714/gi = 1946332/ug = Mm 2025/len = 1250/STRA = for | 470.7 | P | 430.9 | P | 433.9 | P |
| 13 | 103835_f_at | Cluster Incl AF085192: Mus musculus neural visinin-like protein 3 (NVP-3) mRNA, complete cds/cds = (260,841)/ | 916.8 | P | 787 | P | 779.7 | P |
| | | gb = AF085192/gi = 3600082/ug = Mm.20937/len = 1565/STRA = for | | | | | | |
| 13 | 104108_at | Cluster Incl AJ245569: Mus musculus partial mRNA for hypothetical protein (ORF37 DNA)/cds = (218,4009)/gb = AJ245569/ | 240.5 | P | 260.7 | P | 257.8 | P |
| | | gi = 5869933/ug = Mm 21904/len = 4907/STRA = for | | | | | | |
| 13 | 104277_at | Cluster Incl AI834815: UI-M-AM0-ads-a-05-0-UI.s1 Mus musculus cDNA, 3 end/clone = UI-M-AM0-ads-a-05-0-UI/ | 287.8 | P | 109.6 | P | 233.1 | P |
| | | clone_end = 3/gb = AI834815/gi = 5468993/ug = Mm 22218/len = 168/STRA = for | | | | | | |
| 13 | 104333_at | Cluster Incl U69488 Mus musculus viral envelope like protein (G7e) gene, complete cds/cds = (154,1944)/gb = U69488/ | 468.2 | P | 443.2 | P | 487.2 | P |
| | | gi = 2988386/ug = Mm 22506/len = 2910/STRA = for | | | | | | |
| 13 | 96232_at | Cluster Incl AI854281: UI-M-BH0-ajs-d-09-0-UI s1 Mus musculus cDNA, 3 end/clone = UI-M-BH0-ajs-d-09-0-UI/ | 414.6 | P | 414.4 | P | 384.3 | P |
| | | clone_end = 3/gb = AI854281/gi = 5498187/ug = Mm.28776/len = 440/STRA = for | | | | | | |
| 13 | 97374_at | Cluster Incl AI840458 UI-M-AN0-aci-c-06-0-UI s1 Mus musculus cDNA, 3 end/clone = UI-M-AN0-aci-c-06-0-UI/ | 625.4 | P | 329.7 | P | 439.8 | P |
| | | clone_end = 3/gb = AI840458/gi = 5474671/ug = Mm 29721/len = 430/STRA = for | | | | | | |
| 13 | 97401_at | Cluster Incl AW124244 UI-M-BH2 1-aph-f-05-0-UI s1 Mus musculus cDNA, 3 end/clone = UI-M-BH2 1-aph-f-05-0-UI/ | 513.2 | P | 432.8 | P | 542.2 | P |
| | | clone_end = 3/gb = AW124244/gi = 6099774/ug = Mm.29878/len = 381/STRA = for | | | | | | |
| 13 | 97436_at | Cluster Incl AA869653: vq20g06 r1 Mus musculus cDNA, 5 end/clone = IMAGE-1094842/clone_end = 5/gb = AA869653/ | 352.7 | P | 167.7 | P | 99.6 | P |
| | | gi = 2965098/ug = Mm 30110/len = 240/STRA = for | | | | | | |
| 13 | 98459_at | Cluster Incl AA913994: vy91d09.r1 Mus musculus cDNA, 5 end/clone = IMAGE-1313585/clone_end = 5/gb = AA913994/ | 337.9 | P | 262.9 | P | 300.4 | P |
| | | gi = 3053386/ug = Mm 34759/len = 511/STRA = for | | | | | | |
| 13 | 98993_at | Cluster Incl U59418 Mus musculus protein phosphatase 2A B alpha3 regulatory subunit mRNA, partial cds/cds = (406,1671)/ | 875.6 | P | 757.9 | P | 868.1 | P |
| | | gb = U59418/gi = 2429084/ug = Mm 3785/len = 1796/STRA = for | | | | | | |
| 13 | 99546_at | Cluster Incl M77831: Mouse binding protein 13 (Fkbp13) gene exons 1-5, complete cds/cds = (155,577)/gb = M77831/ | 671.5 | P | 726.6 | P | 722.7 | P |
| | | gi = 433782/ug = Mm.4234/len = 740/STRA = for | | | | | | |
| 13 | 100885_at | Cluster Incl AF013166: Mus musculus serine/threonine kinase (nek2) mRNA, complete cds/cds = (57,1388)/gb = AF013166/ | 554.7 | P | 468.1 | P | 463.3 | P |
| | | gi = 2331225/ug = Mm 5379/len = 2869/STRA = for | | | | | | |
| 13 | 101385_at | Cluster Incl AW047312 UI-M-BH1-amh-f-09-0-UI.s1 Mus musculus cDNA, 3 end/clone = UI-M-BH1-amh-f-09-0-UI/ | 1055.1 | P | 901.1 | P | 1216 | P |
| | | clone_end = 3/gb = AW047312/gi = 5907841/ug = Mm.7142/len = 493/STRA = for | | | | | | |
| 13 | 102879_s_at | Cluster Incl M31314 Fc receptor, IgG, high affinity I/cds = (1,1215)/gb = M31314/gi = 200752/ug = Mm.150/len = 1279/ | 666.7 | P | 649.7 | P | 669.5 | P |
| | | STRA = for | | | | | | |
| 13 | 103203_f_at | Cluster Incl AW259499 up28d02.x1 Mus musculus cDNA, 3 end/clone = IMAGE-2655651/clone_end = 3/gb = AW259499/ | 218.7 | P | 176.2 | P | 232 | P |
| | | gi = 6632417/ug = Mm.1911/len = 454/STRA = for | | | | | | |
| 13 | 103442_at | Cluster Incl AI843399 UI-M-AQ1-adz-g-11-0-UI s1 Mus musculus cDNA, 3 end/clone = UI-M-AQ1-adz-g-11-0-UI/ | 333 | P | 218.9 | P | 241.1 | P |
| | | clone_end = 3/gb = AI843399/gi = 5477612/ug = Mm 21475/len = 471/STRA = for | | | | | | |
| 13 | 104491_at | Cluster Incl AI509330 mt94e09.y1 Mus musculus cDNA, 5 end/clone = IMAGE-637576/clone_end = 5/gb = AI509330/ | 715.6 | P | 724.1 | P | 683.5 | P |
| | | gi = 4408235/ug = Mm.3028/len = 360/STRA = for | | | | | | |
| 13 | 92304_at | Cluster Incl D31863 Phosphatidylinositol glycan, class A/cds = (62,1519)/gb = D31863/gi = 1402591/ug = Mm 3781/len = 3534/ | 116.9 | P | 51.7 | P | 199.7 | P |
| | | STRA = for | | | | | | |
| 13 | 92428_at | Cluster Incl AF039405: Mus musculus arsenite-translocating ATPase (ArsA) mRNA, complete cds/cds = (0,1052)/ | 533.1 | P | 428.1 | P | 561 | P |
| | | gb = AF039405/gi = 2745699/ug = Mm 41475/len = 1053/STRA = for | | | | | | |
| 13 | 93670_at | Cluster Incl AW048233 UI-M-BH1-amc-h-10-0-UI s1 Mus musculus cDNA, 3 end/clone = UI-M-BH1-amc-h-10-0-UI/ | 562.8 | P | 572.4 | P | 606.6 | P |
| | | clone_end = 3/gb = AW048233/gi = 5908762/ug = Mm 8068/len = 429/STRA = for | | | | | | |
| 13 | 94788_f_at | Cluster Incl X04663 Mouse mRNA for beta-tubulin (lsotype Mbeta 5)/cds = (78,1412)/gb = X04663/gi = 55047/ug = Mm 1703/ | 5172.3 | P | 3394.1 | P | 3993.2 | P |
| | | len = 1591/STRA = for | | | | | | |
| 13 | 97682_r_at | Cluster Incl J03953 Mouse, glutathione transferase GT9 3 mRNA, 3 end/cds = (0,630)/gb = J03953/gi = 193689/ | 1413.8 | P | 1553.3 | P | 1537 | P |
| | | ug = Mm 37199/len = 1090/STRA = for | | | | | | |
| 13 | 97758_at | Cluster Incl AB023564 Mus musculus gene for type I peroxiredoxin/cds = (11,610)/gb = AB023564/gi = 6467220/ug = Mm 412/ | 2837.5 | P | 3046.4 | P | 3100 | P |
| | | len = 864/STRA = for | | | | | | |
| 13 | 95791_s_at | Cluster Incl U14648 Splicing factor, arginine/serine-rich 10/cds = (0,853)/gb = U14648/gi = 555923/ug = Mm 87815/len = 1772/ | 611.5 | P | 443.5 | P | 530.3 | P |
| | | STRA = for | | | | | | |
| 13 | 97055_s_at | Cluster Incl AI118194: ue34e03 x1 Mus musculus cDNA, 3 end/clone = IMAGE-1482268/clone_end = 3/gb = AI118194/ | 3951.6 | P | 3954.9 | P | 4135.6 | P |
| | | gi = 3518518/ug = Mm 89213/len = 567/STRA = rev | | | | | | |
| 13 | 97061_g_at | Cluster Incl AW215489: up04f11.x1 Mus musculus cDNA, 3 end/clone = IMAGE-2651085/clone_end = 3/gb = AW215489/ | 3725.8 | P | 3673 | P | 3295.6 | P |
| | | gi = 6526165/ug = Mm 89290/len = 522/STRA = rev | | | | | | |
| 13 | 99678_f_at | Cluster Incl AU018994: AU018994 Mus musculus cDNA, 3 end/clone = J0511B11/clone_end = 3/gb = AU018994/ | 753.2 | P | 647.9 | P | 728.1 | P |
| | | gi = 3374497/ug = Mm 95645/len = 265/STRA = rev | | | | | | |
| 13 | 102476_f_at | Cluster Incl AW121303: UI-M-BH2 3-aog-g-01-0-UI s1 Mus musculus cDNA, 3 end/clone = UI-M-BH2.3-aog-g-01-0-UI/ | 799.2 | P | 697.1 | P | 699.4 | P |
| | | clone_end = 3/gb = AW121303/gi = 6096636/ug = Mm48675/len = 217/STRA = rev | | | | | | |
| 13 | 93016_at | Cluster Incl U56243 Mus musculus testis 14-3-3 protein theta-subtype mRNA, complete cds/cds = (20,757)/gb = U56243/ | 475.7 | P | 447.8 | P | 393.3 | P |
| | | gi = 1777958/ug = Mm 14722/len = 761/STRA = for | | | | | | |
| 13 | 93993_at | Cluster Incl AI851062: UI-M-BH0-ajv-h-01-0-UI s1 Mus musculus cDNA, 3 end/clone = UI-M-BH0-ajv-h-01-0-UI/ | 423.6 | P | 336.5 | P | 451.2 | P |
| | | clone_end = 3/gb = AI851062/gi = 5494968/ug = Mm 21752/len = 400/STRA = for | | | | | | |
| 13 | 94071_at | Cluster Incl AI847904: UI-M-AI1-afl-a-09-0-UI s1 Mus musculus cDNA, 3 end/clone = UI-M-AI1-afl-a-09-0-UI/clone_end = 3/ | 163.8 | P | 211.9 | P | 215.3 | P |
| | | gb = AI847904/gi = 5491810/ug = Mm.22114/len = 350/STRA = for | | | | | | |
| 13 | 94882_i_at | Cluster Incl AI850000: UI-M-BG0-aib-e-07-0-UI s1 Mus musculus cDNA, 3 end/clone = UI-M-BG0-aib-e-07-0-UI/ | 952.5 | A | 734.7 | P | 839 | P |
| | | clone_end = 3/gb = AI850000/gi = 5493906/ug = Mm 23693/len = 409/STRA = for | | | | | | |
| 13 | 96081_at | Cluster Incl X60980.Mus musculus TK gene encoding thymidine kinase, exon 1 and 2, partial/cds = (0,97)/gb = X60980/ | 556.1 | P | 191 | P | 142.1 | P |
| | | gi = 54809/ug = Mm.2661/len = 98/STRA = for | | | | | | |
| 13 | 96698_at | Cluster Incl AI835520: UI-M-AI0-aam-d-11-0-UI s1 Mus musculus cDNA, 3 end/clone = UI-M-AI0-aam-d-11-0-UI/ | 376.5 | P | 304.9 | P | 379 | P |
| | | clone_end = 3/gb = AI835520/gi = 5469733/ug = Mm.27558/len = 421/STRA = for | | | | | | |
| 13 | 97295_at | Cluster Incl AW122331: UI-M-BH2 2-aos-f-09-0-UI.s1 Mus musculus cDNA, 3 end/clone = UI-M-BH2.2-aos-f-09-0-UI/ | 364.8 | P | 230.1 | P | 302.4 | P |
| | | clone_end = 3/gb = AW122331/gi = 6097816/ug = Mm 28038/len = 409/STRA = for | | | | | | |
| 13 | 97884_at | Cluster Incl AI844175 UI-M-AL1-ahj-h-09-0-UI s1 Mus musculus cDNA, 3 end/clone = UI-M-AL1-ahj-h-09-0-UI/ | 362.1 | P | 144.1 | P | 236.7 | P |
| | | clone_end = 3/gb = AI844175/gi = 5488081/ug = Mm 28378/len = 319/STRA = for | | | | | | |
| 13 | 98887_at | Cluster Incl AW125607: UI-M-BH2.2-aqj-f-06-0-UI.s1 Mus musculus cDNA, 3 end/clone = UI-M-BH2.2-aqj-f-06-0-UI/ | 535.5 | P | 556.7 | A | 451.1 | P |
| | | clone_end = 3/gb = AW125607/gi = 6101137/ug = Mm.28538/len = 461/STRA = for | | | | | | |
| 13 | 94449_at | Cluster Incl AI854522: UI-M-BH0-ajj-d-05-0-UI.s1 Mus musculus cDNA, 3 end/clone = UI-M-BH0-ajj-d-05-0-UI/clone_end = 3/ | 315.2 | P | 532.9 | P | 351.9 | P |
| | | gb = AI854522/gi = 5498428/ug = Mm 28785/len = 358/STRA = for | | | | | | |
| 13 | 94453_at | Cluster Incl AI852571 UI-M-BH0-aiw-h-10-0-UI s1 Mus musculus cDNA, 3 end/clone = UI-M-BH0-aiw-h-10-0-UI/ | 275.5 | P | 198.7 | P | 177.5 | P |
| | | clone_end = 3/gb = AI852571/gi = 5496477/ug = Mm.28794/len = 293/STRA = for | | | | | | |
| 13 | 96262_at | Cluster Incl AI836812 UI-M-AJ0-abd-b-04-0-UI.s1 Mus musculus cDNA, 3 end/clone = UI-M-AJ0-abd-b-04-0-UI/ | 666.9 | P | 742.9 | A | 617.9 | P |
| | | clone_end = 3/gb = AI836812/gi = 5471025/ug = Mm 29829/len = 289/STRA = for | | | | | | |
| 13 | 96319_at | Cluster Incl AW061324 UI-M-BH1-anw-e-09-0-UI s1 Mus musculus cDNA, 3 end/clone = UI-M-BH1-anw-e-09-0-UI/ | 988.9 | P | 699.6 | P | 736.1 | P |
| | | clone_end = 3/gb = AW061324/gi = 6009075/ug = Mm 29931/len = 401/STRA = for | | | | | | |
| 13 | 97516_at | Cluster Incl U92793 Mus musculus alpha glucosidase II alpha subunit mRNA, complete cds/cds = (11,2911)/gb = U92793/ | 225.6 | P | 208.2 | P | 266.5 | P |
| | | gi = 2104688/ug = Mm 3196/len = 3859/STRA = for | | | | | | |
| 13 | 98076_at | Cluster Incl AI835644 UI-M-AJ0-aau-c-02-0-UI s1 Mus musculus cDNA, 3 end/clone = UI-M-AJ0-aau-c-02-0-UI/ | 660.6 | P | 473.3 | P | 704.2 | P |
| | | clone_end = 3/gb = AI835644/gi = 5469857/ug = Mm 34536/len = 372/STRA = for | | | | | | |
| 13 | 98617_at | Cluster Incl AF071186 Mus musculus WW domain binding protein 11 mRNA, complete cds/cds = (7,1176)/gb = AF071186/ | 497 | P | 566.7 | P | 699 | P |
| | | gi = 3550081/ug = Mm 3855/len = 1864/STRA = for | | | | | | |
| 13 | 99123_s_at | Cluster Incl AW061280 UI-M-BH1-anw-a-07-0-UI s1 Mus musculus cDNA, 3 end/clone = UI-M-BH1-anw-a-07-0-UI/ | 69 | P | 19.8 | P | −52.8 | P |
| | | clone_end = 3/gb = AW061280/gi = 6009031/ug = Mm 4050/len = 155/STRA = for | | | | | | |
| 13 | 100156_at | Cluster Incl D26090 Mouse mRNA for mCDC46 protein, complete cds/cds = (84,2285)/gb = D26090/gi = 940403/ | 525.1 | P | 150.8 | P | 394.8 | P |
| | | ug = Mm 5048/len = 2649/STRA = for | | | | | | |
| 13 | 100559_at | Cluster Incl AI853344.UI-M-BH0-air-b-10-0-UI.s1 Mus musculus cDNA, 3 end/clone = UI-M-BH0-air-b-10-0-UI/ | 199.3 | P | 247.9 | A | 202 | P |
| | | clone_end = 3/gb = AI853344/gi = 5497250/ug = Mm 5624/len = 336/STRA = for | | | | | | |
| 13 | 100564_at | Cluster Incl AF068199 D-dopachrome tautomerase/cds = (0,356)/gb = AF068199/gi = 3452318/ug = Mm 5731/len = 357/ | 268.8 | P | 92.2 | P | 176.3 | P |
| | | STRA = for | | | | | | |
| 13 | 102354_at | Cluster Incl AI049398 ub33c08 r1 Mus musculus cDNA, 5 end/clone = IMAGE-1379534/clone_end = 5/gb = AI049398/ | 371.6 | P | 204.4 | P | 316.4 | P |
| | | gi = 3297685/ug = Mm 11434/len = 420/STRA = for | | | | | | |
| 13 | 102383_at | Cluster Incl AW048977.UI-M-BH1-amo-h-10-0-UI s1 Mus musculus cDNA, 3 end/clone = UI-M-BH1-amo-h-10-0-UI/ | 246.8 | P | 62.9 | P | 153.3 | P |
| | | clone_end = 3/gb = AW048977/gi = 5909506/ug = Mm 12180/len = 470/STRA = for | | | | | | |
| 13 | 103799_at | Cluster Incl AF073881 Mus musculus myotubulann homologous protein 3 mRNA, partial cds/cds = (0,365)/gb = AF073881/ | 88.9 | P | 52.1 | P | 57.6 | P |
| | | gi = 3916225/ug = Mm 20844/len = 940/STRA = for | | | | | | |
| 13 | 103889_at | Cluster Incl AI834833.UI-M-AM0-ads-g-05-0-UI s1 Mus musculus cDNA, 3 end/clone = UI-M-AM0-ads-g-05-0-UI/ | 405.1 | P | 407.6 | P | 320.2 | A |
| | | clone_end = 3/gb = AI834833/gi = 5469046/ug = Mm 21286/len = 407/STRA = for | | | | | | |
| 13 | 95527_at | Cluster Incl AJ132771 Mus musculus mRNA for chromatin assembly factor-1 p150 subunit/cds = (0,2735)/gb = AJ132771/ | 11.7 | P | −43.7 | P | 35.6 | P |
| | | gi = 5852157/ug = Mm 27609/len = 2802/STRA = for | | | | | | |
| 13 | 95541_at | Cluster Incl AW125506: UI-M-BH2.2-aqm-e-08-0-UI.s1 Mus musculus cDNA, 3 end/clone = UI-M-BH2.2-aqm-e-08-0-UI/ | 90 | P | 71.8 | P | 48.4 | P |
| | | clone_end = 3/gb = AW125506/gi = 6101036/ug = Mm 27673/len = 434/STRA = rev | | | | | | |
| 13 | 95634_at | Cluster Incl AI848107 UI-M-AP1-agk-e-09-0-UI s1 Mus musculus cDNA, 3 end/clone = UI-M-AP1-agk-e-09-0-UI/ | 136.1 | P | 106.5 | A | 104.7 | P |
| | | clone_end = 3/gb = AI848107/gi = 5492013/ug = Mm 28094/len = 475/STRA = for | | | | | | |
| 13 | 96753_at | Cluster Incl Y11905 M. musculus BCL7C mRNA/cds = (57,710)/gb = Y11905/gi = 1946223/ug = Mm 2898/len = 781/STRA = for | 173.2 | A | 128 | P | 280.7 | P |
| 13 | 96763_at | Cluster Incl AI839995: UI-M-AH0-acs-f-11-0-UI s1 Mus musculus cDNA, 3 end/clone = UI-M-AH0-acs-f-11-0-UI/ | 150.7 | P | 118 | P | 106 | P |
| | | clone_end = 3/gb = AI839995/gi = 5474208/ug = Mm 29007/len = 357/STRA = for | | | | | | |
| 13 | 98437_at | Cluster Incl U63720 Mus musculus CPP32 apoptotic protease mRNA, partial cds/cds = (0,662)/gb = U63720/gi = 4097524/ | 897.7 | P | 536.8 | P | 493.3 | P |
| | | ug = Mm 34405/len = 1609/STRA = for | | | | | | |
| 13 | 100513_at | Cluster Incl AF075461 Mus musculus ADP-nbosylation factor-directed GTPase activating protein isoform a (Shag1) mRNA, | −63.4 | P | −370.9 | A | 222.5 | P |
| | | complete cds/cds = (49,3492)/gb = AF075461/gi = 4063613/ug = Mm 5237/len = 4370/STRA = rev | | | | | | |
| 13 | 100989_at | Cluster Incl AJ001373 Mus musculus bodenin gene/cds = (173,775)/gb = AJ001373/gi = 2647955/ug = Mm 6818/len = 954/ | 142.3 | P | 123.5 | P | 128.3 | P |
| | | STRA = for | | | | | | |
| 13 | 101854_r_at | Cluster Incl AI844178 UI-M-AL1-ahk-a-02-0-UI s1 Mus musculus cDNA, 3 end/clone = UI-M-AL1-ahl-a-02-0-UI/ | 1213.9 | P | 1018.5 | P | 1117.9 | P |
| | | clone_end = 3/gb = AI844178/gi = 5488084/ug = Mm 866/len = 298/STRA = for | | | | | | |
| 13 | 103711_at | Cluster Incl AI115399: uh85b06.r1 Mus musculus cDNA, 5 end/clone = IMAGE-1764467/clone_end = 5/gb = AI115399/ | 144.1 | P | 90.3 | P | 101.6 | P |
| | | gi = 3515723/ug = Mm 24100/len = 450/STRA = for | | | | | | |
| 13 | 92758_at | Cluster Incl U09268 Mus musculus domesticus 129 tyrosine-threonine dual specificity phosphatase PAC-1 gene, complete | 645 | P | 633.2 | P | 649.8 | P |
| | | cds/cds = (0,539)/gb = U09268/gi = 505624/ug = Mm 4729/len = 540/STRA = for | | | | | | |
| 13 | 93698_at | Cluster Incl AB006329 Mus musculus mSox13 mRNA, complete cds/cds = (48,1835)/gb = AB006329/gi = 3077735/ | 1440.2 | P | 982.5 | P | 1370.7 | P |
| | | ug = Mm 8575/len = 3183/STRA = rev | | | | | | |
| 13 | 95374_f_at | Cluster Incl AI413993 ma76g11.x1 Mus musculus cDNA, 3 end/clone = IMAGE-316676/clone_end = 3/gb = AI413993/ | 1132.5 | P | 814.4 | P | 803.8 | P |
| | | gi = 4257497/ug = Mm 22881/len = 308/STRA = for | | | | | | |
| 13 | AFFX- | L09192 Mus musculus pyruvate carboxylase mRNA, complete cds (_5, _MA, MB, _3 | 315.6 | P | 330.5 | A | 454.5 | P |
| | PyruCarbMur/ | represent transcript regions 5 prime, | | | | | | |
| | L09192_3_at | MiddleA, MiddleB, and 3 prime respectively) | | | | | | |
| 13 | 92879_at | Cluster Incl U42383 Fibroblast growth factor inducible 13/cds = (9,1637)/gb = U42383/gi = 3318654/ug = Mm 14501/ | 513.3 | P | 348.2 | M | 517.6 | P |
| | | len = 2069/STRA = for | | | | | | |
| 13 | 93803_at | Cluster Incl AB007139 Mus musculus Psme3 gene for PA28 gamma subunit, complete cds/cds = (119,883)/gb = AB007139/ | 215.5 | P | 245.4 | P | 226.7 | A |
| | | gi = 3219287/ug = Mm 2141/len = 2555/STRA = for | | | | | | |
| 13 | 94015_at | Cluster Incl AW060447 UI-M-BH1-anj-f-02-0-UI s1 Mus musculus cDNA, 3 end/clone = UI-M-BH1-anj-f-02-0-UI/ | 299.9 | P | 185.3 | P | 287 | P |
| | | clone_end = 3/gb = AW060447/gi = 6006198/ug = Mm 2184/len = 460/STRA = for | | | | | | |
| 13 | 97271_at | Cluster Incl AI845593 UI-M-AQ1-adx-d-04-0-UI.s1 Mus musculus cDNA, 3 end/clone = UI-M-AQ1-adx-d-04-0-UI/ | 283 | P | 240.8 | A | 211 | P |
| | | clone_end = 3/gb = AI845593/gi = 5489499/ug = Mm.27922/len = 390/STRA = for | | | | | | |
| 13 | 98069_s_at | Cluster Incl AI854467 UI-M-BH0-ait-g-02-0-UI s1 Mus musculus cDNA, 3 end/clone = UI-M-BH0-ait-g-02-0-UI/clone_end = 3/ | 161 | A | 168.2 | P | 201.5 | P |
| | | gb = AI854467/gi = 5498373/ug = Mm 34456/len = 390/STRA = for | | | | | | |
| 13 | 101485_at | Cluster Incl AW124859 UI-M-BH2.1-apu-a-02-0-UI.s1 Mus musculus cDNA, 3 end/clone = UI-M-BH2.1-apu-a-02-0-UI/ | 389.5 | P | 319.7 | P | 283.3 | P |
| | | clone_end = 3/gb = AW124859/gi = 6100389/ug = Mm 7861/len = 372/STRA = for | | | | | | |
| 13 | 101967_at | Cluster Incl D50646: Mouse mRNA for SDF2, complete cds/cds = (28,663)/gb = D50646/gi = 1741869/ug = Mm.935/len = 1012/ | 574.4 | A | 509.6 | P | 618.9 | P |
| | | STRA = for | | | | | | |
| 13 | 103884_at | Cluster Incl AI850335: UI-M-BG1-alf-g-11-0-UI.s1 Mus musculus cDNA, 3 end/clone = UI-M-BG1-aif-g-11-0-UI/ | −144.7 | P | −381.8 | A | −205.1 | P |
| | | clone_end = 3/gb = AI850335/gi = 5494241/ug = Mm 21241/len = 475/STRA = for | | | | | | |
| 13 | 104583_at | Cluster Incl AI227492 uf10c07 y1 Mus musculus cDNA, 5 end/clone = IMAGE-1510956/clone_end = 5/gb = AI227492/ | 148.2 | P | 133.8 | A | 180.4 | P |
| | | gi = 3810545/ug = Mm 23975/len = 655/STRA = for | | | | | | |
| 13 | 95612_at | Cluster Incl AA667128 vq89a03.r1 Mus musculus cDNA, 5 end/clone = IMAGE-1109452/clone_end = 5/gb = AA667128/ | 594.7 | P | 470.3 | A | 479.6 | P |
| | | gi = 2625829/ug = Mm.27997/len = 108/STRA = for | | | | | | |
| 13 | 96125_at | Cluster Incl AF110520: Mus musculus major histocompatibllity complex region NG27, NG28, RPS28, NADH | 572.8 | P | 381.4 | M | 642.7 | P |
| | | oxidoreductase, NG29, KIFC1, Fas-binding protein, BING1, tapasin, RalGDS-like, KE2, BING4, beta 1,3-galactosyl | | | | | | |
| | | transferase, and RPS18 genes, complete cds, S/STRA = for | | | | | | |
| 13 | 96234_at | Clusler Incl AI849311, UI-M-AJ1-aha-c-04-0-UI.s1 Mus musculus cDNA, 3 end/clone = UI-M-AJ1-aha-c-04-0-UI/ | 125.8 | P | 102.9 | P | 135 | A |
| | | clone_end = 3/gb = AI849311/gi = 5493217/ug = Mm 28790/len = 471/STRA = for | | | | | | |
| 13 | 100004_at | Cluster Incl AW120890: UI-M-BH2.3-aob-a-12-0-UI s1 Mus musculus cDNA, 3 end/clone = UI-M-BH2 3-aob-a-12-0-UI/ | 491.2 | P | 345.7 | P | 411.5 | A |
| | | clone_end = 3/gb = AW120890/gi = 6096223/ug = Mm 45194/len = 364/STRA = for | | | | | | |
| 13 | 104259_at | Cluster Incl X99641 M musculus mRNA for HP1 alpha protein/cds = (63,638)/gb = X99641/gi = 1480107/ug = Mm 28408/ | 348.7 | P | 270 | A | 271.4 | P |
| | | len = 865/STRA = for | | | | | | |
| 13 | 92332_at | Cluster Incl M80540 Distal-less homeobox 2/cds = (300,1298)/gb = M80540/gi = 201920/ug = Mm 3896/len = 2408/STRA = for | 66.1 | P | −43.7 | P | −2.1 | A |
| 13 | 98355_at | Cluster Incl AA544993-vk42e09 r1 Mus musculus cDNA, 5 end/clone = IMAGE-949288/clone_end = 5/gb = AA544993/ | 534.4 | P | 9.1 | P | 326.9 | P |
| | | gi = 2306067/ug = Mm 4393/len = 297/STRA = for | | | | | | |
| 13 | 99807_r_at | Cluster Incl X97720 Friend virus susceptibility 1/cds = (0,1322)/gb = X97720/gi = 1515301/ug = Mm 57220/len = 1323 | 147.6 | P | 199.1 | P | 207.4 | P |
| | | STRA = for | | | | | | |
| 13 | 100684_at | Cluster Incl U92794-Proteln kinase C substrate 80K-H/cds = (114,1679)/gb = U92794/gi = 2104690/ug = Mm 14244/len = 2030/ | 469.1 | A | 397.9 | P | 476.6 | P |
| | | STRA = for | | | | | | |
| 13 | 101508_at | Cluster Incl U17961: Mus musculus p62 mRNA, complete cds/cds = (111,1442)/gb = U17961/gi = 602250/ug = Mm 8256/ | 230.3 | P | 213.5 | P | 198.4 | P |
| | | len = 1765/STRA = for | | | | | | |
| 13 | 103625_at | Cluster Incl AA797556 vw25a08 r1 Mus musculus cDNA, 5 end/clone = IMAGE-1244822/clone_end = 5/gb = AA797556/ | 295.4 | P | 284.9 | A | 313.8 | A |
| | | gi = 2860511/ug = Mm 20279/len = 427/STRA = for | | | | | | |
| 13 | 104301_at | Cluster Incl AI852414 UI-M-BH0-aip-e-01-0-UI s1 Mus musculus cDNA, 3 end/clone = UI-M-BH0-aip-e-01-0-UI/ | 175.4 | A | 248 | M | 352.6 | P |
| | | clone_end = 3/gb = AI852414/gi = 5496360/ug = Mm.22351/len = 425/STRA = for | | | | | | |
| 13 | 94356_at | Cluster Incl AI593074 vv10f08.x1 Mus musculus cDNA, 3 end/clone = IMAGE-1211267/clone_end = 3/gb = AI593074/ | 77 | A | 19.2 | P | 65.1 | P |
| | | gi = 4602122/ug = Mm 25231/len = 604/STRA = rev | | | | | | |
| 13 | 96189_at | Cluster Incl AW046184 UI-M-BH1-akw-d-10-0-UI s1 Mus musculus cDNA, 3 end/clone = UI-M-BH1-akw-d-10-0-UI/ | 40.2 | P | 10.6 | A | 28 | P |
| | | clone_end = 3/gb = AW046184/gi = 5906713/ug = Mm 28502/len = 416/STRA = for | | | | | | |
| 13 | 99023_at | Cluster Incl U57747 Mus musculus platelet-activating factor acetylhydrolase isoform Ib beta subunit mRNA, complete cds/ | 244.8 | P | 247.1 | P | 291.6 | A |
| | | cds = (106,795)/gb = U57747/gi = 1373362/ug = Mm 3877/len = 944/STRA = for | | | | | | |
| 13 | 100461_at | Cluster Incl D85818 Mouse mRNA for RNA polymerase II subuunit RPB14, complete cds/cds = (16,369)/gb = D85818/ | 420 | A | 336.8 | P | 505.8 | P |
| | | gi = 2081615/ug = Mm 4896/len = 531/STRA = for | | | | | | |
| 13 | 100908_at | Cluster Incl M36033 Protein tyrosine phosphatase, receptor type, A/cds = (25,2514)/gb = M36033/gi = 198876/ug = Mm 587/ | 176 | P | 111.5 | P | 135 | A |
| | | len = 3070/STRA = for | | | | | | |
| 13 | 101856_at | Cluster Incl AI836771.UI-M-AJ0-abc-d-02-0-UI.s1 Mus musculus cDNA, 3 end/clone = UI-M-AJ0-abc-d-02-0-UI/ | 262.4 | P | 157.3 | A | 345.5 | P |
| | | clone_end = 3/gb = AI836771/gi = 5470964/ug = Mm 86933/len = 200/STRA = for | | | | | | |
| 13 | 95765_at | Cluster Incl U48971 Mus musculus class III alcohol dehydrogenase pseudogene/cds = (0,1124)/gb = U48971/gi = 1399469/ | 417.5 | P | 299.9 | A | 450.4 | A |
| | | ug = Mm 87751/len = 1125/STRA = for | | | | | | |
| 13 | 97249_at | Cluster Incl AW124204 UI-M-BH2.1-aph-b-11-0-UI s1 Mus musculus cDNA, 3 end/clone = UI-M-BH2 1-aph-b-11-0-UI/ | 343.4 | A | 202 | A | 302.2 | P |
| | | clone_end = 3/gb = AW124204/gi = 6099734/ug = Mm 27853/len = 382/STRA = for | | | | | | |
| 13 | 95759_at | Cluster Incl AW123746: UI-M-BH2 1-apl-h-09-0-UI s1 Mus musculus cDNA, 3 end/clone = UI-M-BH2 1-apl-h-09-0-UI/ | 38 | M | 22.6 | P | 76 | P |
| | | clone_end = 3/gb = AW123746/gi = 6099276/ug = Mm 298/len = 462/STRA-for | | | | | | |
| 13 | 99139_at | Cluster Incl AW123783 UI-M-BH2 1-apm-d-01-0-UI s1 Mus musculus cDNA, 3 end/clone = UI-M-BH2.1-apm-d-01-0-UI/ | 340.2 | M | 338.6 | A | 359.3 | A |
| | | clone_end = 3/gb = AW123783/gi = 6099313/ug = Mm 41374/len = 404/STRA = for | | | | | | |
| 13 | 101054_at | Cluster Incl X00496 Mouse la-associated invariant chain (II) mRNA fragment/cds = (0,509)/gb = X00496/gi = 53102/ | 210.9 | P | 33.2 | M | 114.7 | M |
| | | ug = Mm.7043/len = 994/STRA = for | | | | | | |
| 13 | 101074_at | Cluster Incl D89063: Mus musculus mRNA for oligosaccharyltransferase, complete cds/cds = (143,1468)/gb = D89063/ | 223.2 | P | 131.1 | A | 135.8 | M |
| | | gi = 2687875/ug = Mm 7236/len = 1750/STRA = for | | | | | | |
| 13 | 103871_at | Cluster Incl AW123729 UI-M-BH2 1-apl-f-11-0-UI s1 Mus musculus cDNA, 3 end/clone = UI-M-BH2 1-apl-f-11-0-UI/ | 7.3 | P | −95.5 | M | −31.7 | P |
| | | clone_end = 3/gb = AW123729/gi = 6099259/ug = Mm 21163/len = 462/STRA = for | | | | | | |
| 13 | 94407_at | Cluster Incl AI835481 UI-M-AQ0-aag-e-02-0-UI s1 Mus muaculus cDNA, 3 end/clone = UI-M-AQ0-aag-e-02-0-UI/ | 206.1 | A | 257.6 | A | 270.8 | P |
| | | clone_end = 3/gb = AI835481/gi = 5469694/ug = Mm 259/len = 220/STRA = for | | | | | | |
| 13 | 96121_at | Cluster Incl AW047866 UI-M-BH1-als-f-05-0-UI s1 Mus musculus cDNA, 3 end/clone = UI-M-BH1-als-f-05-0-UI/ | 340.3 | P | 209.6 | M | 356.4 | P |
| | | clone_end = 3/gb = AW047866/gi = 5908395/ug = Mm 28103/len = 411/STRA = rev | | | | | | |
| 13 | 94644_at | Cluster Incl M99492 Mouse butyrylcholinesterase mRNA, complete cds/cds = (17,1828)/gb = M99492/gi = 191579/ | 38.1 | P | 13 | A | 84 | A |
| | | ug = Mm.8073/len = 1891/STRA = for | | | | | | |
| 13 | 93303_at | Cluster Incl U64445 Ubiquitin fusion degradation 1 —like/cds = (106,1029)/gb = u64445/gi = 1654347/ug = Mm.1830/ | 133.2 | A | 72.5 | A | 157 | A |
| | | len = 1479/STRA = for | | | | | | |
| 13 | 97238_at | Cluster Incl AW209238: uo07g04.x1 Mus musculus cDNA, 3 end/clone = IMAGE-2598870/clone_end = 3/gb = AW209238/ | 65.5 | P | 12.1 | A | 20.1 | A |
| | | gi = 6515178/ug = Mm 27836/len = 568/STRA = for | | | | | | |
| 13 | 97251_at | Cluster Incl AI842124: UI-M-AN1-afg-h-03-0-UI s1 Mus musculus cDNA, 3 end/clone = UI-M-AN1-afg-h-03-0-UI/ | 97.3 | P | 68.7 | A | 81.3 | M |
| | | clone_end = 3/gb = AI842124/gi = 5476337/ug = Mm 27863/len = 301/STRA = for | | | | | | |
| 13 | 100526_f_at | Cluster Incl X84227 Cyritestin 1/cds = (72,2540)/gb = X64227/gi = 1666660/ug = Mm 5168/len = 2722/STRA = for | 188.6 | P | 75.8 | A | 114.3 | M |
| 13 | 94382_at | Cluster Incl AI851901.UI-M-BH0-aix-d-03-0-UI s1 Mus musculus cDNA, 3 end/clone = UI-M-BH0-aix-d-03-0-UI/ | 124.5 | P | 0.1 | A | 111.7 | P |
| | | clone_end = 3/gb = AI851901/gi = 5495807/ug = Mm 25608/len = 428/STRA = for | | | | | | |
| 13 | 99459_f_at | Cluster Incl X70764: ELKL motif kinase/cds = (93,2417)/gb = X70764/gi = 57919/ug = Mm.4082/len = 2763/STRA = for | 75.6 | P | 63.8 | A | 60.1 | A |
| 13 | 100026_at | Cluster Incl U42443: Mus musculus MECA39 mRNA, complete cds/cds = (0,1160)/gb = U42443/gi = 1480454/ug = Mm.4606/ | 341.1 | P | 136.2 | A | 325.5 | P |
| | | len = 1161/STRA = for | | | | | | |
| 13 | 93612_at | Cluster Incl D86332 Matrix metalloproteinase 15/cds = (28,2001)/gb = D86332/gi = 2687872/ug = Mm 7283/len = 3339/ | 62.4 | P | 89.2 | A | 45.2 | M |
| | | STRA = for | | | | | | |
| 13 | 94713_at | Cluster Incl U81453 Myosin VIIa/cds = (10,6657)/gb = U81453/gi = 1778381/ug = Mm 1403/len = 7135/STRA = rev | 342.2 | P | 369.1 | A | 156.8 | A |
| 13 | 95514_at | Cluster Incl AI846302 UI-M-AK1-aez-g-04-0-UI s1 Mus musculus cDNA, 3 end/clone = UI-M-AK1-aez-g-04-0-UI/ | 126.4 | A | −27.8 | M | 64.8 | M |
| | | clone_end = 3/gb = AI846302/gi = 5490208/ug = Mm 253/len = 324/STRA = rev | | | | | | |
| 13 | 104078_g_at | Cluster Incl AW121992 UI-M-BH2 3-aoj-f-11-0-UI.s1 Mus musculus cDNA, 3 end/clone = UI-M-BH2.3-aoj-f-11-0-UI/ | 31 | P | −28.8 | A | 33.4 | A |
| | | clone_end = 3/gb = AW121992/gi = 6097398/ug = Mm 21705/len = 477/STRA = for | | | | | | |
| 13 | 94933_at | Cluster Incl AI853739 UI-M-BH0-ajg-a-12-0-UI.s1 Mus musculus cDNA, 3 end/clone = UI-M-BH0-ajg-a-12-0-UI/ | 294.9 | P | 188.6 | A | 355 | P |
| | | clone_end = 3/gb = AI853739/gi = 5497645/ug = Mm.26783/len = 346/STRA = for | | | | | | |
| 13 | 98493_at | Cluster Incl AA867026 vx22e02 r1 Mus musculus cDNA, 5 end/clone = IMAGE-1265210/clone_end = 5/gb = AA867026/ | 0.6 | A | −48.9 | A | 86 | P |
| | | gi = 2962471/ug = Mm 35606/len = 269/STRA = for | | | | | | |
| 13 | 98994_at | Cluster Incl AF081499 Mus musculus type IIb Na/phosphate-cotransporter (Npt2b) mRNA, complete cds/cds = (44,2137)/ | 72.8 | P | 14.2 | A | 31.5 | A |
| | | gb = AF081499/gi = 3929576/ug = Mm 3786/len = 4039/STRA = for | | | | | | |
| 13 | 99548_at | Cluster Incl AF033034 Mus musculus aldehyde dehydrogenase 3 (aldh3) gene, partial cds/cds = (45,155)/gb = AF033034/ | −282 | M | −273.5 | A | −285 | A |
| | | gi = 2645710/ug = Mm 4257/len = 156/STRA = for | | | | | | |
| 13 | 99961_s_at | Cluster Incl L37092 Mus musculus cyclin-dependent kinase homologue (p130PITSL) mRNA, complete cds/cds = (661,3012)/ | 45.3 | P | −84 | A | 0.9 | A |
| | | gb = L37092/gi = 561745/ug = Mm 4414/len = 3161/STRA = for | | | | | | |
| 13 | 96976_f_at | Cluster Incl X16217 Mouse T11 class I MHC gene (exon 5)/cds = (0,88)/gb = X16217/gi = 54221/ug = Mm 88813/len = 113/ | 355.7 | P | 242.5 | M | 74.5 | A |
| | | STRA = for | | | | | | |
| 14 | 92571_at | Cluster Incl D85904 Mouse mRNA for apg-2, complete cds/cds = (15,2540)/gb = D85904/gi = 1389636/ug = Mm 1032/ | 297.5 | P | 596.5 | P | 417.8 | P |
| | | len = 2582/STRA = for | | | | | | |
| 14 | 92580_at | Cluster Incl U39473 Mus musculus histidyl-tRNA synthetase mRNA, complete cds/cds = (85,1614)/gb = U39473/ | 254.3 | P | 323.4 | P | 300 | P |
| | | gi = 1209682/ug = Mm 10528/len = 1629/STRA = for | | | | | | |
| 14 | 92598_at | Cluster Incl AI843029.UI-M-AK1-aeq-e-08-0-UI s1 Mus musculus cDNA, 3 end/clone = UI-M-AK1-aeq-e-08-0-UI/ | 1044.4 | P | 1112 | P | 1201.1 | P |
| | | clone_end = 3/gb = AI843029/gi = 5477242/ug = Mm 10727/len = 298/STRA = for | | | | | | |
| 14 | 93059_at | Cluster Incl AI854841 UI-M-BH0-akb-b-10-0-UI s1 Mus musculus cDNA, 3 end/clone = UI-M-BH0-akd-b-10-0-UI/ | 379.3 | P | 391.5 | P | 304.1 | P |
| | | clone_end = 3/gb = AI854841/gi = 5498747/ug = Mm 155/len = 396/STRA = for | | | | | | |
| 14 | 93256_at | Cluster Incl AI853728 UI-M-BH0-ajq-e-09-0-UI s1 Mus musculus cDNA, 3 end/clone = UI-M.BH0-ajq-e-09-0-UI/ | 797 | P | 1142.4 | P | 977.9 | P |
| | | clone_end = 3/gb = AI853728/gi = 5497599/ug = Mm 17035/len = 482/STRA = for | | | | | | |
| 14 | 93312_at | Cluster Incl AW124623 UI-M-BH2 1-apw-b-04-0-UI s1 Mus musculus cDNA, 3 end/clone = UI-M-BH2.1-apw-b-4-0-UI/ | 542.2 | P | 639.9 | P | 712.4 | P |
| | | clone_end = 3/gb = AW124623/gi = 6100153/ug = Mm 18485/len = 422/STRA = for | | | | | | |
| 14 | 93319_at | Cluster Incl U20238 Mus musculus GTPase-activating protein GAPIII mRNA, complete cds/cds = (125,2629)/gb-U20238/ | 468 | P | 490.7 | P | 523.7 | P |
| | | gi = 972943/ug = Mm.18517/len = 4107/STRA = for | | | | | | |
| 14 | 93580_at | Cluster Incl AI837010 UI-M-AJ0-aaz-h-06-0-UI s1 Mus musculus cDNA, 3 end/clone = UI-M-AJ0-aaz-h-06-0-UI/ | 714.5 | P | 1056.4 | P | 1035.6 | P |
| | | clone_end = 3/gb = AI837010/gi = 5471223/ug = Mm 20543/len = 472/STRA = for | | | | | | |
| 14 | 94018_at | Cluster Incl AW120725 UI-M-BH2 3-any-a-12-0-UI s1 Mus musculus cDNA, 3 end/clone = UI-M-BH2 3-any-a-12-0-UI/ | 1592.4 | P | 1538.7 | P | 1461.4 | P |
| | | clone_end = 3/gb = AW120725/gi = 6096058/ug = Mm 21846/len = 227/STRA = for | | | | | | |
| 14 | 94245_at | Cluster Incl AW125865 UI-M-BH2 2-aqk-e-12-0-UI s1 Mus musculus cDNA, 3 end/clone = UI-M-BH2 2-aqk-e-12-0-UI/ | 215.1 | P | 214.2 | P | 198.5 | P |
| | | clone_end = 3/gb = AW125865/gi = 6101395/ug = Mm 22362/len = 178/STRA = for | | | | | | |
| 14 | 94834_at | Cluster Incl U06119 Cathepsin H/cds = (47,1048)/gb = U06119/gi = 454100/ug = Mm 2277/len = 1381/STRA = for | 509.8 | P | 474.8 | P | 898.3 | P |
| 14 | 94893_at | Cluster Incl AI840411 UI-M-AH0-acv-h-03-0-UI.s1 Mus musculus cDNA, 3 end/clone = UI-M-AH0-acv-h-03-0-UI/ | 110 | P | 144.6 | P | 174.3 | P |
| | | clone_end = 3/gb = AI840411/gi = 5474624/ug = Mm.23962/len = 435/STRA = for | | | | | | |
| 14 | 95425_at | Cluster Incl U21489 Acetyl coenzyme A dehydrogenase, long-chain/cds = (0,1292)/gb = U21489/gi = 726094/ug = Mm.2445/ | 230.9 | P | 505.7 | P | 489.6 | P |
| | | len = 1434/STRA = for | | | | | | |
| 14 | 96038_at | Cluster Incl AI840339 UI-M-AJ0-abd-g-01-0-UI S2 Mus musculus cDNA, 3 end/clone = UI-M-AJ0-abd-g-01-0-UI/ | 489.9 | P | 636.7 | P | 644.2 | P |
| | | clone_end = 3/gb = AI840339/gi = 5474552/ug = Mm 2587/len = 497/STRA = for | | | | | | |
| 14 | 96079_at | Cluster Incl AI853881 UI-M-BH0-aju-a-01-0-UI s1 Mus musculus cDNA, 3 end/clone = UI-M-BH0-aju-a-01-0-UI/ | 320.4 | P | 397.9 | P | 316.8 | P |
| | | clone_end = 3/gb = AI853881/gi = 5497787/ug = Mm 26598/len = 313/STRA = for | | | | | | |
| 14 | 96088_at | Cluster Incl AB033921 Mus musculus mRNA for Ndr1 related protein Ndr2, complete cds/cds = (35,1150)/gb = AB033921/ | 380.5 | P | 485.6 | P | 490.8 | P |
| | | gi = 6141565/ug = Mm 26722/len = 2010/STRA = for | | | | | | |
| 14 | 96680_at | Cluster Incl AI835630 UI-M-AI0-aap-h-02-0-UI a1 Mus musculus cDNA, 3 end/clone = UI-M-AI0-aap-h-02-0-UI/ | 141.8 | P | 219.7 | P | 202.2 | P |
| | | clone_end = 3/gb = AI835630/gi = 5469843/ug = Mm 27432/len = 327/STRA = for | | | | | | |
| 14 | 97217_at | Cluster Incl AI842095 UI-M-AN1-afg-e-02-0-UI.s1 Mus musculus cDNA, 3 end/clone = UI-M-AN1-afg-e-02-0-UI/ | 1283 | P | 1379.2 | P | 1360.3 | P |
| | | clone_end = 3/gb = AI842095/gi = 5476308/ug = Mm 27733/len = 418/STRA = for | | | | | | |
| 14 | 97241_at | Cluster Incl AI787713 ul21fo4.y1 Mus musculus cDNA, 5 end/clone = IMAGE-2088223/clone_end = 5/gb = AI787713/ | 484.8 | P | 456.7 | P | 508.8 | P |
| | | gi = 5335420/ug = Mm 27839/len = 528/STRA = for | | | | | | |
| 14 | 97802_at | Cluster Incl AI850195: UI-M-BG1-aie-a-03-0-UI.s1 Mus musculus cDNA, 3 end/clone = UI-M-BG1-aie-a-03-0-UI/ | 231.9 | P | 346.6 | P | 283.7 | P |
| | | clone_end = 3/gb = AI850195/gi = 5494101/ug = Mm28128/len = 531/STRA = for | | | | | | |
| 14 | 97868_at | Cluster Incl AW060270.UI-M-BH1-amv-g-11-0-UI s1 Mus musculus cDNA, 3 end/clone = UI-M-BH1-amv-g-11-0-UI/ | 645.8 | P | 801.2 | P | 636.5 | P |
| | | clone_end = 3/gb = AW060270/gi = 6008061/ug = Mm 28330/len = 515/STRA = for | | | | | | |
| 14 | 98880_at | Cluster Incl AW124955-UI-M-BH2 1-apv-a-10-0-UI s1 Mus musculus cDNA, 3 end/clone = UI-M-BH2 1-apv-a-10-0-UI/ | 541.9 | P | 623.5 | P | 594.6 | P |
| | | clone_end = 3/gb = AW124955/gi = 6100485/ug = Mm28524/len = 316/STRA = for | | | | | | |
| 14 | 98931_at | Cluster Incl AW123589: UI-M-BH2 1-api-b-04-0-UI s1 Mus musculus cDNA, 3 end/clone = UI-M-BH2 1-api-b-04-0-UI/ | 267.1 | P | 327.3 | P | 298.5 | P |
| | | clone_end = 3/gb = AW123589/gi = 6099119/ug = Mm 26670/len = 421/STRA = for | | | | | | |
| 14 | 98945_at | Cluster Incl AW125190.UI-M-BH2 1-apz-h-07-0-UI s1 Mus musculus cDNA, 3 end/clone = UI-M-BH2 1-apz-h-07-0-UI/ | 343.2 | P | 358.3 | P | 434.6 | P |
| | | clone_end = 3/gb = AW125190/gi = 6100720/ug = Mm 28714/len = 396/STRA = for | | | | | | |
| 14 | 98950_at | Cluster Incl AB017616 Mus musculus YGR163W mRNA homologue, complete cds/cds = (24,1220)/gb = AB017616/ | 708.2 | P | 728.9 | P | 916.4 | P |
| | | gi = 4519622/ug = Mm 28746/len = 2507/STRA = for | | | | | | |
| 14 | 94469_at | Cluster Incl AW120950: UI-M-BH2.3-aod-b-02-0-UI.s1 Mus musculus cDNA, 3 end/clone = UI-M-BH2.3-aod-b-02-0-UI/ | 790.8 | P | 792.2 | P | 717.6 | P |
| | | clone_end = 3/gb = AW120950/gi = 6096283/ug = Mm.28848/len = 336/STRA = for | | | | | | |
| 14 | 94470_i_at | Cluster Incl AW045974: UI-M-BH1-alc-a-08-0-UI s1 Mus musculus cDNA, 3 end/clone = UI-M-BH1-alc-a-08-0-UI/ | 236.3 | P | 233.5 | P | 250.2 | P |
| | | clone_end = 3/gb = AW045974/gi = 5906503/ug = Mm 28851/len = 338/STRA = for | | | | | | |
| 14 | 94478_at | Cluster Incl AI841377 UI-M-AM0-adv-g-04-0-UI s1 Mus musculus cDNA, 3 end/clone = UI-M-AM0-adv-g-04-0-UI/ | 521.3 | P | 1068.1 | P | 765.5 | P |
| | | clone_end = 3/gb = AI841377/gi = 5475590/ug = Mm.28872/len = 527/STRA = for | | | | | | |
| 14 | 95103_at | Cluster Incl AI839824 UI-M-AN0-acq-c-09-0-UI s1 Mus musculus cDNA, 3 end/clone = UI-M-AN0-acq-c-09-0-UI/ | 600.4 | P | 581 | P | 659.8 | P |
| | | clone_end = 3/gb = AI839824/gi = 5474002/ug = Mm 29348/len = 481/STRA = for | | | | | | |
| 14 | 95695_at | Cluster Incl AB017112 Mus musculus mCAC gene, complete cds/cds = (0,905)/gb = AB017112/gi = 4239973/ug = Mm.29666/ | 364.4 | P | 376.8 | P | 451.4 | P |
| | | len = 906/STRA = for | | | | | | |
| 14 | 95722_at | Cluster Incl AB013137 Mus musculus mRNA for glutaredoxin, complete cds/cds = (49,372)/gb = AB013137/gi = 6451922/ | 1640.6 | P | 1722.7 | P | 1756.2 | P |
| | | ug = Mm 29728/len = 1287/STRA = for | | | | | | |
| 14 | 96840_at | Cluster Incl AI851243 UI-M-BH0-akf-b-01-0-UI s1 Mus musculus cDNA, 3 end/clone = UI-M-BH0-akf-b-01-0-UI/ | 777.5 | P | 997.3 | P | 1059.2 | P |
| | | clone_end = 3/gb = AI851243/gi = 5495149/ug = Mm.30017/len = 430/STRA = for | | | | | | |
| 14 | 96870_at | Cluster Incl AI836740 UI-M-AJ0-aax-h-06-0-UI s1 Mus musculus cDNA, 3 end/clone = UI-M-AJ0-aax-h-06-0-UI/ | 2057.7 | P | 2583.2 | P | 2900.8 | P |
| | | clone_end = 3/gb = AI836740/gi = 5470958/ug = Mm.30065/len = 420/STRA = for | | | | | | |
| 14 | 96900_at | Cluster Incl AW125480 UI-M-BH2 2-aqm-c-05-0-UI s1 Mus musculus cDNA, 3 end/clone = UI-M-BH2 2-aqm-c-05-0-UI/ | 511.6 | P | 658.8 | P | 875 | P |
| | | clone_end = 3/gb = AW125480/gi = 6101010/ug = Mm 30114/len = 476/STRA = for | | | | | | |
| 14 | 97485_at | Cluster Incl AI850953 UI-M-BH0-akd-e-11-0-UI s1 Mus musculus cDNA, 3 end/clone = UI-M-BH0-akd-e-11-0-UI/ | 956.7 | P | 1056.4 | P | 1250.2 | P |
| | | clone_end = 3/gb = AI850953/gi = 5494859/ug = Mm 30849/len = 328/STRA = for | | | | | | |
| 14 | 97549_at | Cluster Incl L29468 Mouse cofilin Isoform mRNA, complete cds/cds = (131,631)/gb = L29468/gi = 498016/ug = Mm 3373/ | 332.9 | P | 365.8 | P | 406 | P |
| | | len = 2974/STRA = for | | | | | | |
| 14 | 99141_at | Cluster Incl U09816 Mus musculus GM2 activator protein (Gm2a) mRNA, complete cds/cds = (56,637)/gb = U09616/ | 491.6 | P | 384.3 | P | 1165.3 | P |
| | | gi = 540017/ug = Mm 4140/len = 2003/STRA = for | | | | | | |
| 14 | 99160_s_at | Cluster Incl AW227647: up12b06.y1 Mus musculus cDNA, 5 end/clone = IMAGE-2654099/clone_end = 5/gb = AW227647/ | 622.1 | P | 834.6 | P | 731.1 | P |
| | | gi = 6556943/ug = Mm 41665/len = 410/STRA = for | | | | | | |
| 14 | 99642_i_at | Cluster Incl X61232: Mouse mRNA for carboxypeptidase H/cds = (39,1472)/gb = X61232/gi = 50312/ug = Mm 43603/ | 804.4 | P | 959.2 | P | 958.5 | P |
| | | len = 1992/STRA = for | | | | | | |
| 14 | 99643_f_at | Cluster Incl X61232: Mouse mRNA for carboxypeptidase H/cds = (39,1472)/gb = X61232/gi = 50312/ug = Mm 43603/ | 804.9 | P | 958 | P | 912.1 | P |
| | | len = 1992/STRA = for | | | | | | |
| 14 | 100042_at | Cluster Incl AI837921 UI-M-AL0-abq-d-08-0-UI s1 Mus musculus cDNA, 3 end/clone = UI-M-AL0-abq-d-08-0-UI/ | 248.1 | P | 287.3 | P | 239.2 | P |
| | | clone_end = 3/gb = AI837921/gi = 5472134/ug = Mm 44240/len = 244/STRA = for | | | | | | |
| 14 | 100120_at | Cluster Incl L17324 Nidogen/cds = (0,751)/gb = L17324/gi = 439608/ug = Mm 4691/len = 752/STRA = for | 330.9 | P | 461.1 | P | 427.8 | P |
| 14 | 100136_at | Cluster Incl M32017.Lysosomal membrane glycoprotein 2/cds = (0,692)/gb = M32017/gi = 198799/ug = Mm 486/len = 1067/ | 336 | P | 766.8 | P | 833.9 | P |
| | | STRA = for | | | | | | |
| 14 | 100151_at | Cluster Incl L29441: Mus musculus mRNA, complete cds/cds = (69,1250)/gb = L29441/gi = 459889/ug = Mm 4962/len = 2029/ | 1103.7 | P | 1335.7 | P | 1187.9 | P |
| | | STRA = for | | | | | | |
| 14 | 100528_at | Cluster Incl AI852278 UI-M-BH0-aje-d-03-0-UI s1 Mus musculus cDNA, 3 end/clone = UI-M-BH0-aje-d-03-0-UI/ | 361.8 | P | 479.4 | P | 439.8 | P |
| | | clone_end = 3/gb = AI852278/gi = 5496184/ug = Mm 5203/len = 185/STRA = for | | | | | | |
| 14 | 101008_at | Cluster Incl AB023485 Mus musculus mRNA for transcription factor CA150b, complete cds/cds = (4,3108)/gb = AB023485/ | 122.1 | P | 176.9 | P | 141.7 | P |
| | | gi = 6329165/ug = Mm 67998/len = 3526/STRA = for | | | | | | |
| 14 | 101039_at | Cluster Incl X04647.Procollagen, type IV, alpha 2/cds = (0,2216)/gb = X04647/gi = 1022322/ug = Mm 6954/len = 3189/ | 1017.2 | P | 1360.3 | P | 1578.8 | P |
| | | STRA = rev | | | | | | |
| 14 | 102049_at | Cluster Incl AJ001418 Mus musculus mRNA for pyruvate dehydrogenase kinase-like protein/cds = (116,1354)/ | 193 | P | 366.3 | P | 440.8 | P |
| | | gb = AJ001418/gi = 3005989/ug = Mm.10283/len = 3449/STRA = for | | | | | | |
| 14 | 102340_at | Cluster Incl AA833077 uc94g09.r1 Mus musculus cDNA, 5 end/clone = IMAGE-1433344/clone_end = 5/gb = AA833077/ | 143 | P | 186.2 | P | 145.6 | P |
| | | gi = 2906805/ug = Mm.11116/len = 379/STRA = for | | | | | | |
| 14 | 102346_at | Cluster Incl AI747755 ul21h12.x1 Mus musculus cDNA, 3 end/clone = IMAGE-2088263/clone_end = 3/gb = AI747755/ | 89.6 | P | 153.2 | P | 150.5 | P |
| | | gi = 5126019/ug = Mm 11218/len = 431/STRA = for | | | | | | |
| 14 | 102370_at | Cluster Incl AA822174: vp36a09.r1 Mus musculus cDNA, 5 end/clone = IMAGE-1078744/clone_end = 5/gb = AA822174/ | 161 | P | 112.7 | P | 220.6 | P |
| | | gi = 2892042/ug = Mm.1187/len = 329/STRA = for | | | | | | |
| 14 | 102827_at | Cluster Incl AW124633 UI-M-BH2 1-apw-c-02-0-UI s1 Mus musculus cDNA, 3 end/clone = UI-M-BH2.1-apw-c-02-0-UI/ | 342.9 | P | 616 | P | 537.8 | P |
| | | clone_end = 3/gb = AW124633/gi = 6100163/ug = Mm.14478/len = 421/STRA = for | | | | | | |
| 14 | 103015_at | Cluster Incl U41465: B-cell leukemia/lymphoma 6/cds = (100,2223)/gb = U41465/gi = 1209719/ug = Mm 15811/len = 2373/ | 350.3 | P | 558.9 | P | 599.2 | P |
| | | STRA = for | | | | | | |
| 14 | 103338_at | Cluster Incl AW120504 UI-M-BH1-ami-f-11-0-UI s2 Mus musculus cDNA, 3 end/clone = UI-M-BH1-ami-f-11-0-UI/ | 112.3 | P | 144.4 | P | 117.9 | P |
| | | clone_end = 3/gb = AW120504/gi = 6095837/ug = Mm.18113/len = 336/STRA = for | | | | | | |
| 14 | 103359_at | Cluster Incl AI854144 UI-M-BH0-ajw-g-04-0-UI s1 Mus musculus cDNA, 3 end/clone = UI-M-BH0-ajw-g-04-0-UI/ | 338.6 | P | 269.9 | P | 360.1 | P |
| | | clone_end = 3/gb = AI854144/gi = 5498050/ug = Mm 18460/len = 515/STRA = for | | | | | | |
| 14 | 103414_at | Cluster Incl AF049850 Mus musculus major histocompatibility locus class III region- complemenl C4 (C4) and cytochrome | 261.7 | P | 500.5 | P | 466.1 | P |
| | | P450 hydroxylase A (CYP21OH-A) genes, complete cds, sip pseudogene, complete sequence, NG6, SKI, and complement | | | | | | |
| | | factor B (Bf) genes, comp/STRA = for | | | | | | |
| 14 | 103796_at | Cluster Incl AF064071: Mus musculus apoptotic protease activating factor 1 mRNA, complete cds/cds = (585,4301)/ | 174.3 | P | 191.5 | P | 223.2 | P |
| | | gb = AF064071/gi = 3694812/ug = Mm.20836/len = 5152/STRA = for | | | | | | |
| 14 | 103893_at | Cluster Incl AW048781 UI-M-BH1-amg-b-03-0-UI s1 Mus musculus cDNA, 3 end/clone = UI-M-BH1-amg-b-03-0-UI/ | 449.6 | P | 513.1 | P | 459.7 | P |
| | | clone_end = 3/gb = AW048781/gi = 5909310/ug = Mm.21295/len = 404/STRA = for | | | | | | |
| 14 | 103901_at | Cluster Incl AW213777 uo42e09 x1 Mus musculus cDNA, 3 end/clone = IMAGE-2645224/clone_end = 3/gb = AW213777/ | 149.6 | P | 150.4 | P | 150.8 | P |
| | | gi = 6520028/ug = Mm.21331/len = 313/STRA = for | | | | | | |
| 14 | 104288_at | Cluster Incl AI853265 UI-M-BH0-aji-c-07-0-UI s1 Mus musculus cDNA, 3 end/clone = UI-M-BH0-aji-c-07-0-UI/clone_end = 3/ | 342.6 | P | 486.6 | P | 465.3 | P |
| | | gb = AI853265/gi = 5497171/ug = Mm 22276/len = 433/STRA = for | | | | | | |
| 14 | 104744_at | Cluster Incl AW046775 UI-M-BH1-alt-h-01-0-UI s1 Mus musculus cDNA, 3 end/clone = UI-M-BH1-alt-h-01-0-UI/ | 221.5 | P | 258.7 | P | 211.7 | P |
| | | clone_end = 3/gb = AW046775/gi = 5907304/ug = Mm 24707/len = 174/STRA = for | | | | | | |
| 14 | 104758_at | Cluster Incl AA895984: vx52h06.r1 Mus musculus cDNA, 5 end/clone = IMAGE-1278875/clone_end = 5/gb = AA895984/ | 104.3 | P | 180.1 | P | 209.3 | P |
| | | gi = 3032377/ug = Mm.24794/len = 339/STRA = for | | | | | | |
| 14 | 94354_at | Cluster Incl AI845514: UI-M-AO1-aek-d-04-0-UI.s1 Mus musculus cDNA, 3 end/clone = UI-M-AO1-aek-d-04-0-UI/ | 305.4 | P | 593.1 | P | 637.1 | P |
| | | clone_end = 3/gb = AI845514/gi = 5489420/ug = Mm 25221/len = 409/STRA = for | | | | | | |
| 14 | 94359_at | Cluster Incl AI849556 UI-M-AH1-agr-b-05-0-UI s1 Mus musculus cDNA, 3 end/clone = UI-M-AH1-agr-b-05-0-UI/ | 898.9 | P | 1107.3 | P | 1005.3 | P |
| | | clone_end = 3/gb = AI849556/gi = 5493462/ug = Mm 25248/len = 396/STRA = rev | | | | | | |
| 14 | 94374_at | Cluster Incl AI850378 UI-M-BG1-aig-d-01-0-UI s1 Mus musculus cDNA, 3 end/clone = UI-M-BG1-aig-d-01-0-UI/ | 280.2 | P | 332.5 | P | 312.4 | P |
| | | clone_end = 3/gb = AI850378/gi = 5494284/ug = Mm.25445/len = 449/STRA = for | | | | | | |
| 14 | 94420_f_at | Cluster Incl AB000777: Cryptochrome 1 (photolyase-like)/cds = (19,1839)/gb = AB000777/gi = 1816438/ug = Mm 26237/ | 155.3 | P | 274.3 | P | 268.1 | P |
| | | len = 2450/STRA = for | | | | | | |
| 14 | 95033_at | Cluster Incl AW049513 UI-M-BH1-anf-b-11-0-UI s1 Mus musculus cDNA, 3 end/clone = UI-M-BH1-anf-b-11-0-UI/ | 341.4 | P | 493.5 | P | 432.8 | P |
| | | clone_end = 3/gb = AW049513/gi = 5910042/ug = Mm 27535/len = 508/STRA = for | | | | | | |
| 14 | 95564_at | Cluster Incl AI117859 uc41d02 r1 Mus musculus cDNA, 5 end/clone = IMAGE-1400547/clone_end = 5/gb = AI117859/ | 101.4 | P | 140.4 | P | 141.4 | P |
| | | gi = 3518183/ug = Mm.27785/len = 328/STRA = for | | | | | | |
| 14 | 95601_at | Cluster Incl AW125420 UI-M-BH2 3-aqh-f-01-0-UI s1 Mus musculus cDNA, 3 end/clone = UI-M-BH2 3-aqh-f-01-0-UI/ | 591.2 | P | 778.3 | P | 667 | P |
| | | clone_end = 3/gb = AW125420/gi = 6100950/ug = Mm 27937/len = 524/STRA = for | | | | | | |
| 14 | 96221_at | Cluster Incl AI606300 vm55g05 y1 Mus musculus cDNA, 5 end/clone = IMAGE-1002200/clone_end = 5/gb = AI606300/ | 125.6 | P | 158.5 | P | 197 | P |
| | | gi = 4615467/ug = Mm 28710/len = 406/STRA = for | | | | | | |
| 14 | 96774_at | Cluster Incl AW047139 UI-M-BH1-alu-c-08-0-UI s1 Mus musculus cDNA, 3 end/clone = UI-M-BH1-alu-c-08-0-UI/ | 686.6 | P | 659 | P | 671.7 | P |
| | | clone_end = 3/gb = AW047139/gi = 5907668/ug = Mm 29048/len = 363/STRA = for | | | | | | |
| 14 | 97406_at | Cluster Incl AW122258 UI-M-BH2 2-aov-g-09-0-UI.s1 Mus musculus cDNA, 3 end/clone = UI-M-BH2.2-aov-g-09-0-UI/ | 347.3 | P | 326.3 | P | 414.7 | P |
| | | clone_end = 3/gb = AW122258/gi = 6097727/ug = Mm 29914/len = 285/STRA = rev | | | | | | |
| 14 | 97985_f_at | Cluster Incl AA709672 vt35h09 r1 Mus musculus cDNA, 5 end/clone = IMAGE-1165121/clone_end = 5/gb = AA709672/ | 761.8 | P | 591 | P | 1050.9 | P |
| | | gi = 2719590/ug = Mm.31297/len = 364/STRA = for | | | | | | |
| 14 | 98478_at | Cluster Incl U95826 Mus musculus cyclin G2 mRNA, complete cds/cds = (123,1157)/gb = U95826/gi = 2149913/ | 322.9 | P | 386.2 | P | 466.2 | P |
| | | ug = Mm 3527/len = 1280/STRA = for | | | | | | |
| 14 | 98505_i_at | Cluster Incl D16333 Mouse mRNA for coproporphyrinogen oxidase, complete cds/cds = (401,1465)/gb = D16333/gi = 436576/ | 338.7 | P | 419.7 | P | 504.1 | P |
| | | ug = Mm 35820/len = 3071/STRA = for | | | | | | |
| 14 | 98506_r_at | Cluster Incl D16333 Mouse mRNA for coproporphyrinogen oxidase, complete cds/cds = (401,1465)/gb = D16333/gi = 436576/ | 205 | P | 274.4 | P | 294 | P |
| | | ug = Mm 35820/len = 3071/STRA = for | | | | | | |
| 14 | 99064_at | Cluster Incl AF026469: Ubiquitous nuclear protein/cds = (50,2938)/gb = AF026469/gi = 2655203/ug = Mm 3974/len = 3329/ | 754.4 | P | 862.6 | P | 780.3 | P |
| | | STRA = for | | | | | | |
| 14 | 99500_at | Cluster Incl U13174: Solute carrier family 12, member 2/cds = (129,3746)/gb = U13174/gi = 3885309/ug = Mm 4168/len = 6504/ | 347.8 | P | 714.1 | P | 511.1 | P |
| | | STRA = rev | | | | | | |
| 14 | 99532_at | Cluster Incl D78382: Mouse DNA for tob family, complete cds/cds = (0,1091)/gb = D78382/gi = 1469158/ug = Mm 4218/ | 1040.2 | P | 1309 | P | 1046.2 | P |
| | | len = 1092/STRA = for | | | | | | |
| 14 | 99960_at | Cluster Incl U18310 SAPK/Erk/kinase 1/cds = (28,1221)/gb = U18310/gi = 1754530/ug = Mm 4412/len = 2292/STRA = for | 239.6 | P | 372.7 | P | 278.4 | P |
| 14 | 100034_at | Cluster Incl U54705: Mus musculus tumor supressor maspin mRNA, complete cds/cds = (189,1316)/gb = U54705/ | 1030.4 | P | 1266.4 | P | 1182.9 | P |
| | | gi = 1490512/ug = Mm 4622/len = 1432/STRA = for | | | | | | |
| 14 | 100475_at | Cluster Incl D63902: Zinc finger protein 147/cds = (9,1913)/gb = D63902/gi = 1088466/ug = Mm.4973/len = 5475/STRA = rev | 246.7 | P | 300.2 | P | 337.4 | P |
| 14 | 100482_at | Cluster Incl AI836408 UI-M-APO-abi-g-08-0-UI s1 Mus musculus cDNA, 3 end/clone = UI-M-AP0-abi-g-08-0-UI/ | 623.3 | P | 1047 | P | 984.5 | P |
| | | clone_end = 3/gb = AI836408/gi = 5470621/ug = Mm 5018/len = 277/STRA = rev | | | | | | |
| 14 | 100978_at | Cluster Incl U62105 FSHD region gene 1/cds = (116,892)/gb = U62105/gi = 1762524/ug = Mm 67/len = 1022/STRA = for | 219.2 | P | 280.1 | P | 311.1 | P |
| 14 | 102870_at | Cluster Incl AW125272: UI-M-BH2 1-aps-h-06-0-UI s1 Mus musculus cDNA, 3 end/clone = UI-M-BH2 1-aps-h-06-0-UI/ | 403 | P | 379.4 | P | 385.4 | P |
| | | clone_end = 3/gb = AW125272/gi = 6100802/ug = Mm 14787/len = 349/STRA = for | | | | | | |
| 14 | 102912_at | Cluster Incl AI265115 uk03d02 y1 Mus musculus cDNA, 5 end/clone = IMAGE-1958307/clone_end = 5/gb = AI265115/ | 414.2 | P | 485.6 | P | 454.2 | P |
| | | gi = 3883273/ug = Mm 1610/len = 473/STRA = for | | | | | | |
| 14 | 103263_at | Cluster Incl AF079755 Mus musculus enhancer of polycomb (Epc1) mRNA, complete cds/cds = (220,2511)/gb = AF079765/ | 462.9 | P | 560.7 | P | 488.2 | P |
| | | gi = 3757891/ug = Mm 20828/len = 3895/STRA = for | | | | | | |
| 14 | 103456_at | Cluster Incl AI841800 UI-M-AN0-acl-g-06-0-UI s1 Mus musculus cDNA, 3 end/clone = UI-M-AN0-acl-g-06-0-UI/ | 386.4 | P | 736.1 | P | 618.9 | P |
| | | clone_end = 3/gb = AI841800/gi = 5476013/ug = Mm 21657/len = 371/STRA = for | | | | | | |
| 14 | 103457_at | Cluster Incl AF083464 Mus musculus DNA polymerase zeta catalytic subunit mRNA, complete cds/cds = (48,9416)/ | 271.3 | P | 284.7 | P | 391.7 | P |
| | | gb = AF083464/gi = 4079830/ug = Mm.2167/len = 10404/STRA = for | | | | | | |
| 14 | 103697_at | Cluster Incl AW061234 UI-M-BH1-amj-b-11-0-UI s2 Mus musculus cDNA, 3 end/clone = UI-M-BH1-emj-b-11-0-UI/ | 473.9 | P | 407 | P | 418.8 | P |
| | | clone_end = 3/gb = AW061234/gi = 6008985/ug = Mm 24003/len = 462/STRA = for | | | | | | |
| 14 | 103773_at | Cluster Incl AW047874 UI-M-BH1-ala-g-02-0-UI s1 Mus musculus cDNA, 3 end/clone = UI-M-BH1-als-g-02-0-UI/ | 196.3 | P | 362.7 | P | 300.5 | P |
| | | clone_end = 3/gb = AW047874/gi = 5908403/ug = Mm.24787/len = 433/STRA = for | | | | | | |
| 14 | 104033_at | Cluster Incl AI841996 UI-M-AH0-ada-b-03-0-UI s1 Mus musculus cDNA, 3 end/clone = UI-M-AH0-ada-b-03-0-UI/ | 219.5 | P | 376.4 | P | 316.7 | P |
| | | clone_end = 3/gb = AI841996/gi = 5476209/ug = Mm 27051/len = 401/STRA = for | | | | | | |
| 14 | 104189_at | Cluster Incl AI122079 uc46e01.r1 Mus musculus cDNA, 5 end/clone = IMAGE-1401048/clone_end = 5/gb = AI122079/ | 154.2 | P | 323.6 | P | 287.5 | P |
| | | gi = 3522403/ug = Mm 27463/len = 362/STRA = for | | | | | | |
| 14 | 104403_at | Cluster Incl AI854118 UI-M-BH0-ajw-d-12-0-UI s1 Mus musculus cDNA, 3 end/clone = UI-M-BH0-ajw-d-12-0-UI/ | 250.4 | P | 392.8 | P | 330.9 | P |
| | | clone_end = 3/gb = AI854118/gi = 5498024/ug = Mm 28691/len = 504/STRA = rev | | | | | | |
| 14 | 104513_at | Cluster Incl AA688938: vs04f09.r1 Mus musculus cDNA, 5 end/clone = IMAGE-1137257/clone_end = 5/gb = AA688938/ | 265.3 | P | 453.2 | P | 434.4 | P |
| | | gi = 2678367/ug = Mm 3090/len = 197/STRA = for | | | | | | |
| 14 | 92217_s_at | Cluster Incl U05265 Glycoprotein 49 B/cds = (43,1050)/gb = U05265/gi = 475446/ug = Mm 34408/len = 1420/STRA = for | 333.8 | P | 383.5 | P | 297.1 | P |
| 14 | 92384_at | Cluster Incl X74351: Xeroderma pigmentosum, complementation group A/cds = (91,909)/gb = X74351/gi = 440584/ | 125.8 | P | 212.5 | P | 143.8 | P |
| | | ug = Mm.4049/len = 938/STRA = for | | | | | | |
| 14 | 92791_at | Cluster Incl AA518588 vh94g10 r1 Mus musculus cDNA, 5 end/clone = IMAGE-901986/clone_end = 5/gb = AA518586/ | 324.9 | P | 306.9 | P | 429.4 | P |
| | | gi = 2258612/ug = Mm. 45985/len = 255/STRA = rev | | | | | | |
| 14 | 93626_at | Cluster Incl AF103875: Mus musculus placenta-specific ATP-binding cassette transporter (Abcp) mRNA, partial cds/ | 193.7 | P | 224.3 | P | 273.4 | P |
| | | cds = (0,443)/gb = AF103875/gi = 4185797/ug = Mm.7458/len = 502/STRA = for | | | | | | |
| 14 | 95387_f_at | Cluster Incl AA266467: mz67g09 r1 Mus musculus cDNA, 5 end/clone = IMAGE-718528/clone_end = 5/gb = AA266467/ | 328.2 | P | 366.4 | P | 428.3 | P |
| | | gi = 1902637/ug = Mm 23470/len = 372/STRA = rev | | | | | | |
| 14 | 95897_at | Cluster Incl AW061228 UI-M-BH1-amj-a-12-0-UI s2 Mus musculus cDNA, 3 end/clone = UI-M-BH1-amj-a-12-0-UI/ | 176.5 | P | 307.2 | P | 181.1 | P |
| | | clone_end = 3/gb = AW061228/gi = 6008979/ug = Mm.24179/len = 475/STRA = rev | | | | | | |
| 14 | 98855_r_at | Cluster Incl AA014745 mh18b05 r1 Mus musculus cDNA, 5 end/clone = IMAGE-442833/clone_end = 5/gb = AA014745/ | 183.5 | P | 236.9 | P | 249.6 | P |
| | | gi = 1475626/ug = Mm 5096/len = 189/STRA = for | | | | | | |
| 14 | 95785_s_at | Cluster Incl Y13361 RAB7, member RAS oncogene family, pseudogene 1/cds = (0,623)/gb = Y13361/gi = 2168154/ | 317 | P | 389.1 | P | 339.1 | P |
| | | ug = Mm 87807/len = 624/STRA = for | | | | | | |
| 14 | 101221_at | Cluster Incl C76746 C76746 Mus musculus cDNA, 3 end/clone = J0018D06/clone_end = 3/gb = C76746/gi = 2517076/ | 90.5 | P | 192.7 | P | 243.4 | P |
| | | ug = Mm.5529/len = 574/STRA = rev | | | | | | |
| 14 | 93836_at | Cluster Incl AF041054 Mus musculus E1B 19K/Bcl-2-binding protein homolog (Nip3) mRNA, nuclear gene encoding | 240.7 | P | 299.5 | P | 432.1 | P |
| | | mitochondrial protein, complete cds/cds = (83,646)/gb = AF041054/gi = 2773344/ug = Mm 2159/len = 1311/STRA = for | | | | | | |
| 14 | 96106_at | Cluster Incl AW049122 UI-M-BH1-amt-h-11-0-UI s1 Mus musculus cDNA, 3 end/clone = UI-M-BH1-amt-h-11-0-UI/ | 146.7 | P | 85.4 | A | 243.6 | P |
| | | clone_end = 3/gb = AW049122/gi = 5909651/ug = Mm 26903/len = 513/STRA = for | | | | | | |
| 14 | 96679_at | Cluster Incl AW120711: UI-M-BH2 3-anz-e-10-0-UI.s1 Mus musculus cDNA, 3 end/clone = UI-M-BH2.3-anz-e-10-0-UI/ | 30.1 | P | 69.6 | P | 51.9 | P |
| | | clone_end = 3/gb = AW120711/gi = 6096009/ug = Mm.27432/len = 497/STRA = for | | | | | | |
| 14 | 96884_at | Cluster Incl AI847631 UI-M-AP1-agg-e-12-0-UI s1 Mus musculus cDNA, 3 end/clone = UI-M-AP1-agg-e-12-0-UI/ | 376.7 | P | 445.8 | P | 534.5 | P |
| | | clone_end = 3/gb = AI847631/gi = 5491537/ug = Mm.30081/len = 334/STRA = for | | | | | | |
| 14 | 97475_at | Cluster Incl D38517.Mouse mRNA for Dhm1 protein, complete cds/cds = (0,2843)/gb = D38517/gi = 1060920/ug = Mm 3065/ | 103.7 | P | 85.6 | P | 96.3 | P |
| | | len = 2844/STRA = for | | | | | | |
| 14 | 103673_at | Cluster Incl M57891: Complement component 2 (wthin H-2S)/cds = (52,2334)/gb = M57891/gi = 192436/ug = Mm 2081/ | 183.5 | M | 188.8 | P | 221.2 | P |
| | | len = 2587/STRA = for | | | | | | |
| 14 | 104569_at | Cluster Incl AW122024 UI-M-BH2.2-aoo-a-11-0-UI s1 Mus musculus cDNA, 3 end/clone = UI-M-BH2 2-aoo-a-11-0-UI/ | 159.1 | A | 235 | P | 236.1 | P |
| | | clone_end = 3/gb = AW122024/gi = 6097430/ug = Mm 2389/len = 498/STRA = for | | | | | | |
| 14 | 104600_at | Cluster Incl AI841692 UI-M-AL0-abo-a-02-0-UI s1 Mus musculus cDNA, 3 end/clone = UI-M-AL0-abo-a-02-0-UI/ | 69.8 | P | 95.8 | P | 94.2 | P |
| | | clone_end = 3/gb = AI841692/gi = 5475905/ug = Mm 24084/len = 415/STRA = for | | | | | | |
| 14 | 95031_at | Cluster Incl AW060212: UI-M-BH1-amu-f-06-0-UI.s1 Mus musculus cDNA, 3 end/clone = UI-M-BH1-amu-f-06-0-UI/ | 64.4 | P | 88.5 | P | 60.7 | M |
| | | clone_end = 3/gb = AW060212/gi = 6007963/ug = Mm 27520/len = 378/STRA = for | | | | | | |
| 14 | 97972_at | Cluster Incl AB012161 Mus musculus gene for KF-1, complete cds/cds = (0,2051)/gb = AB012161/gi = 4521316/ug = Mm 310/ | 133.1 | P | 158.1 | P | 145.8 | P |
| | | len = 2052/STRA = for | | | | | | |
| 14 | 99018_at | Cluster Incl AA874446 vx09g08 r1 Mus musculus cDNA, 5 end/clone = IMAGE-1263998/clone_end = 5/gb = AA874446/ | 56.6 | P | 91.1 | P | 75.9 | P |
| | | gi = 2979135/ug = Mm 385/len = 511/STRA = for | | | | | | |
| 14 | 99512_at | Cluster Incl AI852647: UI-M-BH0-aiu-h-07-0-UI s1 Mus musculus cDNA, 3 end/clone = UI-M-BH0-aiu-h-07-0-UI/ | 555.2 | P | 653 | P | 825.1 | P |
| | | clone_end = 3/gb = AI852647/gi = 5496553/ug = Mm 41858/len = 379/STRA = for | | | | | | |
| 14 | 103678_at | Cluster Incl AI841139 UI-M-AH0-acz-g-08-0-UI s1 Mus musculus cDNA, 3 end/clone = UI-M-AH0-acz-g-08-0-UI/ | 255 | P | 310.9 | P | 478.7 | P |
| | | clone_end = 3/gb = AI841139/gi = 5475352/ug = Mm 23879/len = 450/STRA = for | | | | | | |
| 14 | 104671_at | Cluster Incl D88994 AMP deaminase 3/cds = (5,2083)/gb = D88994/gi = 3493348/ug = Mm 3238/len = 2127/STRA = for | 87.6 | P | 188.6 | P | 166.6 | P |
| 14 | 93148_at | Cluster Incl AI851013 UI-M-BH0-ajv-c-07-0-UI s1 Mus musculus cDNA, 3 end/clone = UI-M-BH0-ajv-c-07-0-UI/ | 151.5 | P | 213.6 | P | 172.8 | P |
| | | clone_end = 3/gb = AI851013/gi = 5494919/ug = Mm 5231/len = 485/STRA = for | | | | | | |
| 14 | 98802_at | Cluster Incl D30782 Mouse mRNA for epiregulin, complete cds/cds = (138,626)/gb = D30782/gi = 1255631/ug = Mm 4791/ | 112 | P | 115.3 | P | 261.1 | P |
| | | len = 4141/STRA = for | | | | | | |
| 14 | 94066_at | Cluster Incl AW123517 UI-M-BH2 1-aqb-c-09-0-UI s1 Mus musculus cDNA, 3 end/clone = UI-M-BH2 1-aqb-c-09-0-UI/ | 360.2 | P | 303.1 | P | 374.1 | P |
| | | clone_end = 3/gb = AW123517/gi = 6099047/ug = Mm 22086/len = 244/STRA = for | | | | | | |
| 14 | 95093_at | Cluster Incl AI035334 ue18f10x1 Mus musculus cDNA, 3 end/clone = IMAGE-1480747/clone_end = 3/gb = AI035334/ | 165.7 | A | 266.3 | P | 212.5 | A |
| | | gi = 3259044/ug = Mm.29303/len = 182/STRA = for | | | | | | |
| 14 | 103011_at | Cluster Incl L36831: Transcriptional regulator, SIN3 yeast homolog A/cds = (78,3926)/gb = L36831/gi = 556218/ | 361.8 | P | 376.4 | A | 377.3 | P |
| | | ug = Mm 15755/len = 4721/STRA = for | | | | | | |
| 14 | 101370_at | Cluster Incl U20619.Mus musculus serine-rich RNA polymerase suppressor protein 1 (SRP1) mRNA, complete sequence/ | 69.3 | P | 325.4 | P | 240.9 | P |
| | | cds = UNKNOWN/gb = U20619/gi = 3046878/ug = Mm 6952/len = 3882/STRA = for | | | | | | |
| 14 | 102916_s_at | Cluster Incl AB010266 Mus musculus Creb-rp, Tnx and Cyp21 genes for cAMP response element binding protein-related | 294.9 | P | 182.6 | A | 915.8 | P |
| | | protein, lenascin-X and steroid 21-hydroxylase, partial cds and complete sequences/cds = (0,12344)/gb = AB010266/ | | | | | | |
| | | gi = 2804287/ug = Mm.16163/len = /STRA = for | | | | | | |
| 14 | 102922_at | Cluster Incl AI851387.UI-M-BH0-akh-g-12-0-UI s1 Mus musculus cDNA, 3 end/clone = UI-M-BH0-akh-g-12-0-UI/ | 94.8 | P | 105.9 | P | 58.6 | A |
| | | clone_end = 3/gb = AI851387/gi = 5495293/ug = Mm 1630/len = 436/STRA = rev | | | | | | |
| 14 | 102966_at | Cluster Incl AA934166 vt48h11.r1 Mus musculus cDNA, 5 end/clone = IMAGE-1166373/clone_end = 5/gb = AA934166/ | 225.4 | P | 205.4 | P | 221.2 | P |
| | | gi = 3091323/ug = Mm 1819/len = 321/STRA = for | | | | | | |
| 14 | 103254_at | Cluster Incl AW049897: UI-M-BH1-anr-h-08-0-UI s1 Mus musculus cDNA, 3 end/clone = UI-M-BH1-anr-h-08-0-UI/ | 201.3 | P | 140.4 | A | 175.3 | P |
| | | clone_end = 3/gb = AW049897/gi = 5910426/ug = Mm 20488/len = 275/STRA = for | | | | | | |
| 14 | 104661_at | Cluster Incl AF031164: AF031164 Mus musculus cDNA/gb = AF031164/gi = 4887815/ug = Mm.31787/len = 800/STRA = for | 120.6 | P | 170.7 | P | 122.6 | P |
| 14 | 93846_at | Cluster Incl U82324 Mus musculus A6 protein tyrosine kinase homolog mRNA, complete cds/cds = (56,1108)/gb = U82324/ | 86.6 | A | 153.3 | P | 108 | P |
| | | gi = 1769576/ug = Mm 7958/len = 1200/STRA = for | | | | | | |
| 14 | 97172_s_at | Cluster Incl D86037: Sulfonylurea receptor 2/cds = (161,4801)/gb = D86037/gi = 4996851/ug = Mm 35070/len = 6210/ | 55.4 | P | 49.3 | P | 69.4 | P |
| | | STRA = for | | | | | | |
| 14 | 96692_at | Cluster Incl AA759408 vw64c05.r1 Mus musculus cDNA, 5 end/clone = IMAGE-1248584/clone_end = 5/gb = AA759408/ | 164.7 | A | 337.9 | P | 196.2 | P |
| | | gi = 2807202/ug = Mm.27523/len = 424/STRA = for | | | | | | |
| 14 | 101538_i_at | Cluster Incl AW226939 um64a07.y1 Mus musculus cDNA, 5 end/clone = IMAGE-2286132/clone_end = 5/gb = AW226939/ | 166.1 | P | 286.2 | P | 303.8 | P |
| | | gi = 6556235/ug = Mm 88078/len = 486/STRA = rev | | | | | | |
| 14 | 102836_at | Cluster Incl U96724: Putative phosphatase/cds = (111,1517)/gb = U96724/gi = 2121245/ug = Mm 1458/len = 2684/STRA = for | 225.5 | P | 219.4 | P | 357.7 | P |
| 14 | 94980_at | Cluster Incl AI006319: ua70b05.r1 Mus musculus cDNA, 5 end/clone = IMAGE-1362801/clone_end = 5/gb = AI006319/ | 77.6 | A | 107.4 | A | 136.6 | P |
| | | gi = 3215928/ug = Mm.27275/len = 381/STRA = for | | | | | | |
| 14 | 103716_at | Cluster Incl AI851218: UI-M-BH0-ajx-g-06-0-UI s1 Mus musculus cDNA, 3 end/clone = UI-M-BH0-ajx-g-06-0-UI/ | 201.6 | A | 283.9 | P | 209.1 | P |
| | | clone_end = 3/gb = AI851218/gi = 5495124/ug = Mm.24160/len = 306/STRA = rev | | | | | | |
| 14 | 103967_at | Cluster Incl AI551105 vx37f03 x1 Mus musculus cDNA, 3 end/clone = IMAGE-1277405/clone_end = 3/gb = AI551105/ | 56.4 | P | 84.7 | P | 50.9 | A |
| | | gi = 4483468/ug = Mm.25678/len = 390/STRA = for | | | | | | |
| 14 | 95292_at | Cluster Incl AA189389 mt85f02 r1 Mus musculus cDNA, 5 end/clone = IMAGE-636699/clone_end = 5/gb = AA189389/ | 65.7 | A | 71.3 | A | 57.5 | P |
| | | gi = 1776990/ug = Mm 1919/len = 451/STRA = for | | | | | | |
| 14 | 97253_at | Cluster Incl AI854343 UI-M-BG1-aic-c-07-0-UI s1 Mus musculus cDNA, 3 end/clone = UI-M-BG1-aic-c-07-0-UI/ | 115.4 | P | 168.2 | A | 137.5 | P |
| | | clone_end = 3/gb = AI854343/gi = 5498249/ug = Mm 27872/len = 305/STRA = for | | | | | | |
| 14 | 102032_at | Cluster Incl AW060819: UI-M-BH1-amx-b-03-0-UI.s1 Mus musculus cDNA, 3 end/clone = UI-M-BH1-amx-b-03-0-UI/ | 184.3 | P | 285 | A | 291.6 | P |
| | | clone_end = 3/gb = AW060819/gi = 6008570/ug = Mm 10153/len = 507/STRA = for | | | | | | |
| 14 | 102780_at | Cluster Incl Z31362: M musculus (Balb/C) Tx01 mRNA/cds = (0,159)/gb = Z31362/gi = 467582/ug = Mm 1364/len = 228/ | 137.6 | P | 197.7 | P | 268.4 | P |
| | | STRA = for | | | | | | |
| 14 | 102852_at | Cluster Incl M31131: Cadhenn 2/cds = (332,3052)/gb = M31131/gi = 192327/ug = Mm 14897/len = 4321/STRA = for | 43.2 | P | 51.6 | P | 40.6 | P |
| 14 | 104065_at | Cluster Incl AW212878 uo68g01 x1 Mus musculus cDNA, 3 end/clone = IMAGE-2647728/clone_end = 3/gb = AW212878/ | 40.9 | P | 39.5 | A | 50.8 | A |
| | | gi = 6519026/ug = Mm 21596/len = 280/STRA = for | | | | | | |
| 14 | 104067_at | Cluster Incl AI648999 uk33h10.x1 Mus musculus cDNA, 3 end/clone = IMAGE-1970851/clone_end = 3/gb = AI648999/ | 116.9 | P | 86.6 | P | 94.3 | A |
| | | gi = 4729833/ug = Mm.21608/len = 579/STRA = for | | | | | | |
| 14 | 104144_at | Cluster Incl AW124369 UI-M-BH2 1-ape-a-04-0-UI.s1 Mus musculus cDNA, 3 end/clone = UI-M-BH2 1-ape-a-04-0-UI/ | 238.1 | A | 263.8 | P | 169.8 | P |
| | | clone_end = 3/gb = AW124369/gi = 6099899/ug = Mm 22147/len = 214/STRA = for | | | | | | |
| 14 | 97984_i_at | Cluster Incl AA709672: vt35h09 r1 Mus musculus cDNA, 5 end/clone = IMAGE-1165121/clone_end = 5/gb = AA709672/ | 170.4 | P | 137.5 | M | 166.1 | P |
| | | gi = 2719590/ug = Mm.31297/len = 364/STRA = for | | | | | | |
| 14 | 104215_at | Cluster Incl AA592069.vo14b08.r1 Mus musculus cDNA, 5 end/clone = IMAGE-1049847/clone_end = 5/gb = AA592069/ | 53.9 | P | 49.8 | P | 83.8 | P |
| | | gi = 2405732/ug = Mm.27843/len = 329/STRA = for | | | | | | |
| 14 | 98297_at | Cluster Incl AF110818 Mus musculus orphan G protein-coupled receptor FEX mRNA, complete cds/cds = (0,2723)/ | 150.2 | P | 217.7 | P | 247.2 | P |
| | | gb = AF110818/gi = 4262545/ug = Mm.42103/len = 3115/STRA = for | | | | | | |
| 14 | 93054_at | Cluster Incl AI846368 UI-M-AN1-afa-e-09-0-UI s1 Mus musculus cDNA, 3 end/clone = UI-M-AN1-afa-e-09-0-UI/ | −1.2 | M | 132.9 | A | 131.1 | A |
| | | clone_end = 3/gb = AI846368/gi = 5490274/ug = Mm 15351/len = 385/STRA = for | | | | | | |
| 14 | 94324_f_at | Cluster Incl U49878.3-hydroxy-3-methylglutaryl-coenzyme A lyase/cds = (34,1011)/gb = U49878/gi = 1292951/ | 588.7 | A | 661.3 | P | 481.8 | A |
| | | ug = Mm 22668/len = 1440/STRA = for | | | | | | |
| 14 | 96912_s_at | Cluster Incl X15591 Cytotoxic T lymphocyte-associated protein 2 alpha/cds = (6,416)/gb = X15591/gi = 50588/ug = Mm.30144/ | 489 | P | 336.1 | A | 612.1 | P |
| | | len = 664/STRA = for | | | | | | |
| 14 | 99166_at | Cluster Incl AI845963 UI-M-AP1-agl-a-09-0-UI s1 Mus musculus cDNA, 3 end/clone = UI-M-AP1-agl-a-09-0-UI/ | 314.4 | A | 271.6 | A | 390.2 | A |
| | | clone_end = 3/gb = AI845963/gi = 5489869/ug = Mm 41683/len = 223/STRA = for | | | | | | |
| 14 | 96811_at | Cluster Incl AW049806 UI-M-BH1-anm-g-08-0-UI s1 Mus musculus cDNA, 3 end/clone = UI-M-BH1-anm-g-08-0-UI/ | 4.3 | A | 250.8 | P | 178.3 | P |
| | | clone_end = 3/gb = AW04986/gi = 5910335/ug = Mm 29274/len = 457/STRA = for | | | | | | |
| 14 | 99510_at | Cluster Incl X59274 M musculus mRNA brain protein kinase C beta-1/cds = UNKNOWN/gb = X59274/gi = 50185/ | 106.3 | A | 226.9 | A | 181.4 | P |
| | | ug = Mm 4182/len = 2493/STRA = for | | | | | | |
| 14 | 101848_g_at | Cluster Incl AF040242 untitled/cds = (0,240)/gb = AF040242/gi = 3309551/ug = Mm 8551/len = 241/STRA = for | 165.4 | A | 165.5 | A | 145.5 | P |
| 14 | 100321_f_at | Cluster Incl AA407120 EST02104 Mus musculus cDNA, 5 end/clone = C0017F03/clone_end = 5/gb = AA407120/ | 17.8 | A | 39.6 | P | 28.2 | M |
| | | gi = 2067019/ug = Mm 87656/len = 359/STRA = for | | | | | | |
| 14 | 101804_at | Cluster Incl AF032715 Mus musculus type II peroxiredoxin protein 3 (PrxII-3) pseudogene, complete sequence/ | 177.2 | M | 296.8 | A | 316.6 | P |
| | | cds = (0,452)/gb = AF032715/gi = 3603232/ug = Mm.57146/len = 453/STRA = for | | | | | | |
| 14 | 94491_at | Cluster Incl AW123194: UI-M-BH2 1-apg-c-01-0-UI s1 Mus musculus cDNA, 3 end/clone = UI-M-BH2.1-apg-c-01-0-UI/ | 84.1 | P | 112.6 | A | 121.3 | A |
| | | clone_end = 3/gb = AW123194/gi = 6098764/ug = Mm 28912/len = 509/STRA = for | | | | | | |
| 14 | 95094_g_at | Cluster Incl AI035334 ue18f10 x1 Mus musculus cDNA, 3 end/clone = IMAGE-1480747/clone_end = 3/gb = AI035334/ | 36 | A | 101.7 | P | 93.1 | P |
| | | gi = 3259044/ug = Mm.29303/len = 182/STRA = for | | | | | | |
| 14 | 104368_at | Cluster Incl U51204: Mus musculus APC-binding protein EB2 mRNA, partial cds/cds = (0,677)/gb = U51204/gi = 1256435/ | 69.7 | A | 73.1 | A | 205.8 | P |
| | | ug = Mm 22628/len = 1540/STRA = for | | | | | | |
| 14 | 96770_at | Cluster Incl AA607761 vo08f08 r1 Mus musculus cDNA, 5 end/clone = IMAGE-1040871/clone_end = 5/gb = AA607761/ | −70.9 | A | −90.6 | P | 29 | P |
| | | gi = 2455196/ug = Mm 29017/len = 491/STRA = for | | | | | | |
| 14 | 100507_at | Cluster Incl Y09257 M musculus mRNA for NOV protein/cds = (32,1096)/gb = Y09257/gi = 1666472/ug = Mm 5167/len = 2364/ | −380.5 | A | 191.4 | A | 51.3 | A |
| | | STRA = for | | | | | | |
| 14 | 102965_at | Cluster Incl AW121646 UI-M-BH2 3-aoc-b-03-0-UI s1 Mus musculus cDNA, 3 end/clone = UI-M-BH2 3-aoc-b-03-0-UI/ | 304.6 | A | 379.9 | A | 337.1 | P |
| | | clone_end = 3/gb = AW121646/gi = 6096979/ug = Mm.1806/len = 512/STRA = for | | | | | | |
| 14 | 93609_at | Cluster Incl AA797709 vw31c03 r1 Mus musculus cDNA, 5 end/clone = IMAGE-1245412/clone_end = 5/gb = AA797709/ | 62.8 | P | 89.9 | A | 23.5 | P |
| | | gi = 2860664/ug = Mm 7265/len = 464/STRA = for | | | | | | |
| 14 | 96528_at | Cluster Incl D18912 MUSGS01097 Mus musculus cDNA, 3 end/clone = mc0837/clone_end = 3/gb = D18912/gi = 1089548/ | 26.5 | A | 50.9 | P | 50.5 | A |
| | | ug = Mm 27078/len = 254/STRA = rev | | | | | | |
| 14 | 97710_f_at | Cluster Incl AI425990: mg38e07 x1 Mus musculus cDNA, 3 end/clone = IMAGE-426084/clone_end = 3/gb = AI425990/ | 37.9 | A | 30.5 | A | 52.2 | P |
| | | gi = 4271921/ug = Mm 38264/len = 407/STRA = rev | | | | | | |
| 15 | AFFX-BioDn- | E Coli BioD 3 prime | 2877.6 | P | 4819 | P | 4310.2 | P |
| | 3_at | | | | | | | |
| 15 | AFFX-CreX- | Cre 5 prime | 4063.7 | P | 7014.9 | P | 6238.6 | P |
| | 5_at | | | | | | | |
| 15 | AFFX-CreX- | Cre 3 prime | 2585.3 | P | 4814.9 | P | 4414.9 | P |
| | 3_at | | | | | | | |
| 15 | AFFX-b- | M12481 Mouse cytoplasmic beta-actin mRNA (_5, _M, _3 represent transcript regions 5 prime, | 3594.4 | P | 3862.3 | P | 4355.7 | P |
| | ActinMur/ | Middle, and 3 prime respectively) | | | | | | |
| | M12481_5_at | | | | | | | |
| 15 | AFFX-b- | M12481 Mouse cytoplasmic beta-actin mRNA (_5, _M, _3 represent transcript regions 5 prime, | 4787.8 | P | 4615.7 | P | 5572 | P |
| | ActinMur/ | Middle, and 3 prime respectively) | | | | | | |
| | M12481_M_{—} | | | | | | | |
| | at | | | | | | | |
| 15 | AFFX-b- | M12481 Mouse cytoplasmic beta-actin mRNA (_5, _M, _3 represent transcript regions 5 prime, | 3295.4 | P | 3165.6 | P | 4288.1 | P |
| | ActinMur/ | Middle, and 3 prime respectively) | | | | | | |
| | M12481_3_{—} | | | | | | | |
| | at | | | | | | | |
| 15 | AFFX- | M32599 Mouse glyceraldehyde-3-phosphate dehydrogenase mRNA, complete cds (_5, _M, _3 represent | 4693.8 | P | 5637.5 | P | 5744.9 | P |
| | GapdhMur/ | transcript regions 5 prime, Middle, and 3 prime respectively) | | | | | | |
| | M32599_5_at | | | | | | | |
| 15 | AFFX- | M32599 Mouse glyceraldehyde-3-phosphate dehydrogenase mRNA, complete cds (_5, _M, _3 represent | 5025.9 | P | 5397.7 | P | 5325 | P |
| | GapdhMur/ | transcript regions 5 prime, Middle, and 3 prime respectively) | | | | | | |
| | M32599_M_{—} | | | | | | | |
| | at | | | | | | | |
| 15 | AFFX- | M32599 Mouse glyceraldehyde-3-phosphate dehydrogenase mRNA, complete cds (_5, _M, _3 represent | 3352.7 | P | 3980.8 | P | 4332.6 | P |
| | GapdhMur/ | transcript regions 5 prime, Middle, and 3 prime respectively) | | | | | | |
| | M32599_3_at | | | | | | | |
| 15 | AFFX- | X63136 M musculus DNA for intragenic sequence Including B2 element | 2616.8 | P | 2693.9 | P | 2506.6 | P |
| | MUR_b2_at | | | | | | | |
| 15 | AFFX- | U01310 Mus musculus C57/Black6 BC1 scRNA | 3979.6 | P | 3692.7 | P | 4501.5 | P |
| | MURINE_{—} | | | | | | | |
| | b1_{—at} | | | | | | | |
| 15 | AFFX- | K00131 mouse b2 repeat sequence from clone mm61 | 2090.5 | P | 3591.4 | P | 3607.5 | P |
| | MURINE_{—} | | | | | | | |
| | B2_{—at} | | | | | | | |
| 15 | 92541_at | Cluster Incl X12973 Murine MLC1F/MLC3F gene for myosin alkali light chain (exon 1) (last skeletal muscle isoform)/ | 4061.1 | P | 4421.3 | P | 4670.6 | P |
| | | cds = (125,238)/gb = X12973/gi = 53140/ug = Mm.1000/len = 250/STRA = for | | | | | | |
| 15 | 92543_at | Cluster Incl AW123542 UI-M-BH2.1-aqb-f-01-0-UI.s1 Mus musculus cDNA, 3 end/clone = UI-M-BH2.1-aqb-f-01-0-UI/ | 347 | P | 249.8 | P | 339.3 | P |
| | | clone_end = 3/gb = AW123542/gi = 6099072/ug = Mm 100400/len = 373/STRA = for | | | | | | |
| 15 | 92577_f_at | Cluster Incl c UI-M-BH1-alu-a-05-0-UI s1 Mus musculus cDNA, 3 end/clone = UI-M-BH1-alu-a-05-0-UI/clone_end = 3/ | 5850 | P | 6267.2 | P | 6266.7 | P |
| | | gb = AW047116/gi = 5907645/ug = Mm 10474/len = 309/STRA = for | | | | | | |
| 15 | 92624_r_at | Cluster Incl U09189: Mus musculus Ioricrin gene, complete cds/cds = (23,1468)/gb = U09189/gi = 520479/ug = Mm 1121/ | 3631.8 | P | 3262.2 | P | 4024.6 | P |
| | | len = 1704/STRA = for | | | | | | |
| 15 | 92628_at | Cluster Incl X75895 M musculus mRNA for ribosomal protein L36/cds = (20,337)/gb = X75895/gi = 440801/ug = Mm 11376/ | 5908.8 | P | 5592 | P | 6333.9 | P |
| | | len = 387/STRA = for | | | | | | |
| 15 | 92629_f_at | Cluster Incl D63707 Mouse mRNA for hepatoma derived growth factor (HDGF), complete cds/cds = (61,774)/gb = D63707/ | 3339.3 | P | 3806.8 | P | 3583.9 | P |
| | | gi = 945418/ug = Mm 1141/len = 1563/STRA = for | | | | | | |
| 15 | 92631_f_at | Cluster Incl M19380 Calmodulin 3/cds = (109,558)/gb = M19380/gi = 469419/ug = Mm 1147/len = 722/STRA = for | 754.9 | P | 653.3 | P | 992.7 | P |
| 15 | 92783_at | Cluster Incl X02891 Growth hormone/cds = (61,711)/gb = X02891/gi = 51067/ug = Mm.1240/len = 777/STRA = for | 939.2 | P | 1013.3 | P | 859.7 | P |
| 15 | 92880_at | Cluster Incl M38337 Milk fat globule-EGF factor 8 protein/cds = (45,1436)/gb = M38337/gi = 199142/ug = Mm 1451/len = 2077/ | 1361.7 | P | 1423.4 | P | 2140.8 | P |
| | | STRA = for | | | | | | |
| 15 | 92881_at | Cluster Incl U77943 Mus musculus myosin light chain 2 mRNA, complete cds/cds = (0,509)/gb = U77943/gi = 1675395/ | 2193 | P | 2438.3 | P | 2600.3 | P |
| | | ug = Mm 14526/len = 580/STRA = for | | | | | | |
| 15 | 93030_at | Cluster Incl AI844457: UI-M-AJ1-ahe-c-02-0-UI s1 Mus musculus cDNA, 3 end/clone = UI-M-AJ1-ahe-c-02-0-UI/ | 3763.9 | P | 4180.9 | P | 4105.8 | P |
| | | clone_end = 3/gb = AI844457/gi = 5488363/ug = Mm 14833/len = 347/STRA = for | | | | | | |
| 15 | 93038_f_at | Cluster Incl M69260: Lipocortin 1/cds = (14,1054)/gb = M69260/gi = 198844/ug = Mm.14860/len = 1340/STRA = for | 3682.3 | P | 4142.2 | P | 4075.2 | P |
| 15 | 93076_at | Cluster Incl AW124171: UI-M-BH2 1-apr-h-07-0-UI s1 Mus musculus cDNA, 3 end/clone = UI-M-BH2 1-apr-h-07-0-UI/ | 2777.5 | P | 2680.7 | P | 2421.3 | P |
| | | clone_end = 3/gb = AW124171/gi = 6099701/ug = Mm 1581/len = 173/STRA = for | | | | | | |
| 15 | 93077_s_at | Cluster Incl D86232 Lymphocyte antigen 6 complex, locus C/cds = (144,452)/gb = D86232/gi = 1695689/ug = Mm 1583/ | 1727.5 | P | 2131.1 | P | 2642.5 | P |
| | | len = 2683/STRA = for | | | | | | |
| 15 | 93101_s_at | Cluster Incl U96635 Neural precursor cell expressed, developmentally down-regulated gene 4/cds = (611,2944)/gb = U96635/ | 1745.2 | P | 2070.7 | P | 1883.4 | P |
| | | gi = 2088622/ug = Mm 16553/len = 5581/STRA = for | | | | | | |
| 15 | 93116_at | Cluster Incl J02626 Protein kinase, cAMP dependent, catalytic, beta/cds = (192,1247)/gb = J02826/gi = 200386/ | 831.7 | P | 1116.4 | P | 1278 | P |
| | | ug = Mm 16766/len = 1272/STRA = for | | | | | | |
| 15 | 93271_s_at | Cluster Incl AJ245401: Mus musculus Nesp gene for neuroendocrine secretory peptide 55/cds = (0,761)/gb = AJ245401/ | 3430 | P | 4470 | P | 4863.7 | P |
| | | gi = 6010255/ug = Mm 17446/len = 762/STRA = for | | | | | | |
| 15 | 93324_at | Cluster Incl M58566 Mouse TIS11 primary response gene, complete cds/cds = (67,1083)/gb = M58566/gi = 202064/ | 2950.2 | P | 2466 | P | 2527.2 | P |
| | | ug = Mm 18571/len = 2659/STRA = for | | | | | | |
| 15 | 93534_at | Cluster Incl X53929 Decorin/cds = (231,1295)/gb = X53929/gi = 53668/ug = Mm 1987/len = 1372/STRA = for | 4755.6 | P | 5440 | P | 5250.9 | P |
| 15 | 93535_at | Cluster Incl AF041472: Mus musculus ataxin-2 (SCA2) mRNA, complete cds/cds = (26,3883)/gb = AF041472/gi = 3005019/ | 393.6 | P | 512.3 | P | 571.5 | P |
| | | ug = Mm 1989/len = 4225/STRA = for | | | | | | |
| 15 | 93543_f_at | Cluster Incl J03952 Mouse, glutathione transferase GT8 7 mRNA, complete cds/cds = (12,668)/gb = J03952/gi = 193687/ | 1291 | P | 1061.3 | P | 1238.6 | P |
| | | ug = Mm 2011/len = 1018/STRA = for | | | | | | |
| 15 | 93604_f_at | Cluster Incl AF061260 Mus musculus immunosuperfamily protein BI2 mRNA, complete cds/cds = (308,1195)/ | 1687.9 | P | 1802.7 | P | 2129.4 | P |
| | | gb = AF061260/gi = 3779241/ug = Mm 20921/len = 1861/STRA = for | | | | | | |
| 15 | 93730_at | Cluster Incl AF085809 Mus musculus synapsin lb (Synl) mRNA, complete cds/cds = (135,2147)/gb = AF085809/gi = 3599472/ | 3432.2 | P | 3379.1 | P | 3716 | P |
| | | ug = Mm 20936/len = 3694/STRA = for | | | | | | |
| 15 | 93740_at | Cluster Incl U33196 Mus musculus Y-box binding protein mYB-1a mRNA, complete cds/cds = (115,1083)/gb = U33196/ | 3849 | P | 3691.7 | P | 3723.3 | P |
| | | gi = 988280/ug = Mm 21054/len = 1194/STRA = for | | | | | | |
| 15 | 93744_at | Cluster Incl AI119347 ue98e05.y1 Mus musculus cDNA, 5 end/clone = IMAGE-1499168/clone_end = 5/gb = AI119347/ | 2475.7 | P | 2906.7 | P | 3172.7 | P |
| | | gi = 3519671/ug = Mm 21075/len = 476/STRA = for | | | | | | |
| 15 | 93748_at | Cluster Incl AW123901.UI-M-BH2 1-app-g-06-0-UI.s1 Mus musculus cDNA, 3 end/clone = UI-M-BH2 1-app-g-06-0-UI/ | 1438.1 | P | 1344.2 | P | 1421 | P |
| | | clone_end = 3/gb = AW123901/gi = 6099431/ug = Mm 21094/len = 226/STRA = for | | | | | | |
| 15 | 93762_at | Cluster Incl AI841493 UI-M-AH0-acy-d-06-0-UI s1 Mus musculus cDNA, 3 end/clone = UI-M-AH0-acy-d-06-0-UI/ | 1275.1 | P | 1448.1 | P | 1453.3 | P |
| | | clone_end = 3/gb = AI841493/gi = 5475706/ug = Mm 21158/len = 367/STRA = for | | | | | | |
| 15 | 93991_at | Cluster Incl X07295 Malate dehydrogenase, mitochondrial/cds = (0,1016)/gb = X07295/gi = 50679/ug = Mm.21743/len = 1017/ | 3877.4 | P | 4122 | P | 4308.4 | P |
| | | STRA = for | | | | | | |
| 15 | 94068_at | Cluster Incl AW048899 UI-M-BH1-amf-h-03-0-UI s1 Mus musculus cDNA, 3 end/clone = UI-M-BH1-amf-h-03-0-UI/ | 2925.6 | P | 3204.2 | P | 3335.7 | P |
| | | clone_end = 3/gb = AW048899/gi = 5909428/ug = Mm 22103/len = 481/STRA = for | | | | | | |
| 15 | 94247_at | Cluster Incl AA600542: vm75a03 r1 Mus musculus cDNA, 5 end/clone = IMAGE-1004044/clone_end = 5/gb = AA600542/ | 4742.3 | P | 4995.5 | P | 5045.6 | P |
| | | gi = 2434167/ug = Mm 22365/len = 140/STRA = for | | | | | | |
| 15 | 94305_at | Cluster Incl U03419: Mus musculus alpha-1 type I procollagen mRNA, partial cds/cds = (0,977)/gb = U03419/gi = 424103/ | 3340.6 | P | 4335.2 | P | 4615.2 | P |
| | | ug = Mm.22621/len = 1205/STRA = for | | | | | | |
| 15 | 94321_at | Cluster Incl V00830 Keratin complex 1, acidic, gene 10/cds = (27,1739)/gb = V00830/gi = 52786/ug = Mm.22662/len = 1944/ | 3673.2 | P | 4001.2 | P | 4137.9 | P |
| | | STRA = for | | | | | | |
| 15 | 94823_at | Cluster Incl AI835315 UI-M-AQ0-aah-a-06-0-UI s1 Mus musculus cDNA, 3 end/clone = UI-M-AQ0-aah-a-06-0-UI/ | 5102.8 | P | 5447.8 | P | 5708.7 | P |
| | | clone_end = 3/gb = AI835315/gi = 5469528/ug = Mm 22723/len = 275/STRA = for | | | | | | |
| 15 | 94839_at | Cluster Incl M96823: Mouse nucleobindin mRNA, complete cds/cds = (18,1385)/gb = M96823/gi = 200113/ug = Mm.2283/ | 634.2 | P | 651.4 | P | 629.3 | P |
| | | len = 2116/STRA = for | | | | | | |
| 15 | 94845_at | Cluster Incl AW125736: UI-M-BH2.2-aqi-g-02-0-U1.s1 Mus musculus cDNA, 3 end/clone = UI-M-BH2.2-aqi-g-02-0-Ul/ | 1182.7 | P | 1361.3 | P | 1387.7 | P |
| | | clone_end = 3/gb = AW125736/gi = 6101231/ug = Mm.23014/len = 426/STRA = for | | | | | | |
| 15 | 95435_at | Cluster Incl AI844009 UI-M-AL1-ahi-a-05-0-UI s1 Mus musculus cDNA, 3 end/clone = UI-M-AL1-ahi-a-05-0-UI/ | 340.6 | P | 335.7 | P | 286.8 | P |
| | | clone_end = 3/gb = AI844009/gi = 5487910/ug = Mm 24499/len = 408/STRA = for | | | | | | |
| 15 | 95440_at | Cluster Incl AI846093 UI-M-AK1-aew-c-02-0-UI.s1 Mus musculus cDNA, 3 end/clone = UI-M-AK1-aew-c-02-0-UI/ | 222.3 | P | 226 | P | 184.3 | P |
| | | clone_end = 3/gb = AI846093/gi = 5489964/ug = Mm 24564/len = 284/STRA = for | | | | | | |
| 15 | 95458_s_at | Cluster Incl AW121960 UI-M-BH2 3-aoi-h-09-0-UI s1 Mus musculus cDNA, 3 end/clone = UI-M-BH2 3-aoi-h-09-0-UI/ | 1860.7 | P | 1706.4 | P | 1754.7 | P |
| | | clone_end = 3/gb = AW121960/gi = 6097311/ug = Mm 24703/len = 370/STRA = for | | | | | | |
| 15 | 95490_at | Cluster Incl AW120891: UI-M-BH2 3-aob-b-02-0-UI s1 Mus musculus cDNA, 3 end/clone = UI-M-BH2.3-aob-b-02-0-UI/ | 1361.5 | P | 1583.6 | P | 1631.5 | P |
| | | clone_end = 3/gb = AW120891/gi = 6096224/ug = Mm 25045/len = 178/STRA = for | | | | | | |
| 15 | 96018_r_at | Cluster Incl AI853561 UI-M-BH0-ajd-h-09-0-UI s1 Mus musculus cDNA, 3 end/clone = UI-M-BH0-ajd-h-09-0-UI/ | 366 | P | 427.4 | P | 330.1 | P |
| | | clone_end = 3/gb = AI853561/gi = 5497467/ug = Mm 25634/len = 480/STRA = for | | | | | | |
| 15 | 96066_s_at | Cluster Incl X97047: Pyruvate kinase 3/cds = (6,1601)/gb = X97047/gi = 1405932/ug = Mm.2635/len = 2134/STRA = for | 3582 | P | 3789.4 | P | 4223 | P |
| 15 | 96072_at | Cluster Incl M17516: Mouse lactate dehydrogenase A-4 (LDH-A) mRNA, complete cds/cds = (0,395)/gb = M17516/ | 3681.3 | P | 4398.9 | P | 4769.8 | P |
| | | gi = 198776/ug = Mm.26504/len = 881/STRA = for | | | | | | |
| 15 | 96699_at | Cluster Incl X53476 High mobility group protein 14/cds = (33,323)/gb = X53476/gi = 51334/ug = Mm.2756/len = 1070/ | 5614.7 | P | 5854.9 | P | 6056.3 | P |
| | | STRA = for | | | | | | |
| 15 | 97229_at | Cluster Incl AW061042: UI-M-BH1-akk-b-09-0-Ul s2 Mus musculus cDNA, 3 end/clone = UI-M-BH1-akk-b-09-0-UI/ | 1476.5 | P | 1833.1 | P | 1834 | P |
| | | clone_end = 3/gb = AW061042/gi = 6008793/ug = Mm 27796/len = 502/STRA = for | | | | | | |
| 15 | 97808_at | Cluster Incl AI844532 UI-M-AJ1-ahf-d-05-0-UI.s2 Mus musculus cDNA, 3 end/clone = UI-M-AJ1-ahf-d-05-0-UI/ | 1411.4 | P | 1130.4 | P | 1202.7 | P |
| | | clone_end = 3/gb = AI844532/gi = 5488438/ug = Mm 28150/len = 521/STRA = for | | | | | | |
| 15 | 97825_at | Cluster Incl AI854029 UI-M-BH0-ajm-c-04-0-UI s1 Mus musculus cDNA, 3 end/clone = UI-M-BH0-ajm-c-04-0-UI/ | 5684.7 | P | 5617.7 | P | 5824.2 | P |
| | | clone_end = 3/gb = AI854029/gi = 5497935/ug = Mm 28209/len = 528/STRA = for | | | | | | |
| 15 | 98922_at | Cluster Incl L34260 Integral membrane protein 1/cds = (111,2228)/gb = L34260/gi = 508542/ug = Mm 2863/len = 3094/ | 116.3 | P | 149 | P | 129.7 | P |
| | | STRA = for | | | | | | |
| 15 | 98927_at | Cluster Incl AI851048: UI-M-BH0-ajv-f-08-0-UI s1 Mus musculus cDNA, 3 end/clone = UI-M-BH0-ajv-f-08-0-UI/ | 1035.3 | P | 1315.4 | P | 1179.7 | P |
| | | clone_end = 3/gb = AI851048/gi = 5494954/ug = Mm 28650/len = 129/STRA = for | | | | | | |
| 15 | 94464_at | Cluster Incl AF029347 Mus musculus chloride channel protein 3 (CLCN3) mRNA, complete cds/cds = (0,2456)/ | 208.7 | P | 281.3 | P | 275.5 | P |
| | | gb = AF029347/gi = 2599549/ug = Mm.28842/len = 2457/STRA = for | | | | | | |
| 15 | 94481_at | Cluster Incl AI851321 UI-M-BH0-akh-a-06-0-UI s1 Mus musculus cDNA, 3 end/clone = UI-M-BH0-akh-a-06-0-UI/ | 455.9 | P | 618.7 | P | 545.6 | P |
| | | clone_end = 3/gb = AI851321/gi = 5495227/ug = Mm 28877/len = 420/STRA = for | | | | | | |
| 15 | 94536_s_at | Cluster Incl AI843417: UI-M-AQ1-aec-a-10-0-UI s1 Mus musculus cDNA, 3 end/clone = UI-M-AQ1-aec-a-10-0-UI/ | 3247.8 | P | 3275.4 | P | 3399.4 | P |
| | | clone_end = 3/gb = AI843417/gi = 5477630/ug = Mm 29057/len = 345/STRA = for | | | | | | |
| 15 | 95046_s_at | Cluster Incl M98036 Mus musculus guanine nucleotide exchange factor delta subunit (JGR1A) mRNA, complete cds/ | 473.4 | P | 613.2 | P | 504.3 | P |
| | | cds = (69,1643)/gb = M98036/gi = 529429/ug = Mm 29128/len = 1677/STRA = for | | | | | | |
| 15 | 95064_at | Cluster Incl AI849271 UI-M-AJ1-agz-g-06-0-UI s1 Mus musculus cDNA, 3 end/clone = UI-M-AJ1-agz-g-06-0-UI/ | 4137.6 | P | 3823.9 | P | 4629.4 | P |
| | | clone_end = 3/gb = AI849271/gi = 5493177/ug = Mm 29165/len = 405/STRA = rev | | | | | | |
| 15 | 95072_at | Cluster Incl AW121892 UI-M-BH2.3-aoi-e-12-0-UI s1 Mus musculus cDNA, 3 end/clone = UI-M-BH2 3-aoi-e-12-0-UI/ | 2904.4 | P | 3055.9 | P | 3396.9 | P |
| | | clone_end = 3/gb = AW121892/gi = 6097266/ug = Mm 29196/len = 438/STRA = for | | | | | | |
| 15 | 95124_i_at | Cluster Incl AW122337: UI-M-BH2.2-aos-g-04-0-UI s1 Mus musculus cDNA, 3 end/clone = UI-M-BH2 2-aos-g-04-0-UI/ | 1406.8 | P | 1424.8 | P | 1325.6 | P |
| | | clone_end = 3/gb = AW122337/gi = 6097822/ug = Mm 29405/len = 403/STRA = for | | | | | | |
| 15 | 95125_f_at | Cluster Incl AW122337: UI-M-BH2 2-aos-g-04-0-UI s1 Mus musculus cDNA, 3 end/clone = UI-M-BH2 2-aos-g-04-0-UI/ | 2080.5 | P | 2213.5 | P | 2039 | P |
| | | clone_end = 3/gb = AW122337/gi = 6097822/ug = Mm 29405/len = 403/STRA = for | | | | | | |
| 15 | 95132_r_at | Cluster Incl AI852592 UI-M-BH0-aiu-b-11-0-UI s1 Mus musculus cDNA, 3 end/clone = UI-M-BH0-aiu-b-11-0-UI/ | 1638.8 | P | 1347.2 | P | 1830.7 | P |
| | | clone_end = 3/gb = AI852592/gi = 5496498/ug = Mm 29415/len = 392/STRA = rev | | | | | | |
| 15 | 95661_at | Cluster Incl L08115: CD9 antigen/cds = (54,734)/gb = L08115/gi = 388911/ug = Mm 2956/len = 1130/STRA = for | 2965.6 | P | 3319.5 | P | 3099.3 | P |
| 15 | 95698_at | Cluster Incl AI851810: UI-M-BH0-ais-c-02-0-UI s1 Mus musculus cDNA, 3 end/clone = UI-M-BH0-ais-c-02-0-UI/ | 1929.8 | P | 1567.7 | P | 1980 | P |
| | | clone_end = 3/gb = AI851810/gi = 5495716/ug = Mm 29683/len = 276/STRA = for | | | | | | |
| 15 | 95705_s_at | Cluster Incl J04181: Mouse A-X actin mRNA complete cds/cds = (62,1189)/gb = J04181/gi = 191660/ug = Mm 297/len = 1857/ | 3547.8 | P | 4189.1 | P | 3758.8 | P |
| | | STRA = for | | | | | | |
| 15 | 95706_at | Cluster Incl X16834 Mouse mRNA for Mac-2 antigen/cds = (43,837)/gb = X16834/gi = 52986/ug = Mm 2970/len = 955/ | 2741.8 | P | 2455 | P | 2874.9 | P |
| | | STRA = for | | | | | | |
| 15 | 95713_at | Cluster Incl D67067 Mouse mRNA for thymic epithelial cell surface antigen, complete cds/cds = (12,1559)/gb = D67067/ | 1662.4 | P | 1666.9 | P | 1951.4 | P |
| | | gi = 2059325/ug = Mm 29710/len = 2446/STRA = for | | | | | | |
| 15 | 95723_r_at | Cluster Incl AI841464: UI-M-AH0-acy-a-04-0-UI.s1 Mus musculus cDNA, 3 end/clone = UI-M-AH0-acy-a-04-0-UI/ | 2882.6 | P | 2974.6 | P | 3093.9 | P |
| | | clone_end = 3/gb = AI841464/gi = 5475677/ug = Mm.29731/len = 413/STRA = for | | | | | | |
| 15 | 95740_at | Cluster Incl AA727291: vu95a10 r1 Mus musculus cDNA, 5 end/clone = IMAGE-1209882/clone_end = 5/gb = AA727291/ | 4011.5 | P | 4416 | P | 4953 | P |
| | | gi = 2744998/ug = Mm.29753/len = 279/STRA = for | | | | | | |
| 15 | 95755_at | Cluster Incl D14485: Mouse mRNA for dbpA murine homologue, complete cds/cds = (183,1109)/gb = D14485/gi = 1160330/ | 3011 | P | 3112 | P | 3121.2 | P |
| | | ug = Mm 29794/len = 1647/STRA = for | | | | | | |
| 15 | 96290_f_at | Cluster Incl U93863: Mus musculus ribosomal protein L21 mRNA, complete cds/cds = (36,518)/gb = U93863/gi = 1938403/ | 5848.5 | P | 5858.7 | P | 6379.8 | P |
| | | ug = Mm 29896/len = 612/STRA = for | | | | | | |
| 15 | 96298_f_at | Cluster Incl AF020185: Mus musculus protein inhibitor of nitric oxide synthase (PIN) mRNA, complete cds/cds = (99,388)/ | 5536.8 | P | 6411.7 | P | 6386.8 | P |
| | | gb = AF020185/gi = 4103058/ug = Mm.29908/len = 531/STRA = for | | | | | | |
| 15 | 96300_f_at | Cluster Incl AI8542318 UI-M-BH0-ajz-g-10-0-UI s1 Mus musculus cDNA, 3 end/clone = UI-M-BH0-ajz-g-10-0-UI/ | 4313.8 | P | 4322.2 | P | 4391.3 | P |
| | | clone_end = 3/gb = AI854238/gi = 5498144/ug = Mm.29911/len = 349/STRA = for | | | | | | |
| 15 | 96307_s_at | Cluster Incl AW121947 UI-M-BH2 3-aoj-f-05-0-UI s1 Mus musculus cDNA, 3 end/clone = UI-M-BH2 3-aoj-f-05-0-UI/ | 3111 | P | 3644.9 | P | 3730.2 | P |
| | | clone_end = 3/gb = AW121947/gi = 6097393/ug = Mm.29919/len = 347/STRA = for | | | | | | |
| 15 | 96327_at | Cluster Incl AI852535: UI-M-BH0-aiw-e-05-0-UI.s1 Mus musculus cDNA, 3 end/clone = UI-M-BH0-aiw-e-05-0-UI/ | 779.5 | P | 642 | P | 719.1 | P |
| | | clone_end = 3/gb = AI852535/gi = 5496441/ug = Mm 29962/len = 357/STRA = for | | | | | | |
| 15 | 96358_at | Cluster Incl AI837403: UI-M-AP0-abm-e-02-0-UI.s1 Mus musculus cDNA, 3 end/clone = UI-M-AP0-abm-e-02-0-UI/ | 2955.2 | P | 3448 | P | 3337.9 | P |
| | | clone_end = 3/gb = AI837403/gi = 5471616/ug = Mm.30011/len = 404/STRA = for | | | | | | |
| 15 | 96859_at | Cluster Incl AB026621: Mus musculus mRNA for RIE2. complete cds/cds = (481,2895)/gb = AB026621/gi = 5931597/ | 2072.7 | P | 1827 | P | 2339.1 | P |
| | | ug = Mm 30051/len = 3110/STRA = for | | | | | | |
| 15 | 96872_at | Cluster Incl AI835846: UI-M-AI0-aat-b-06-0-UI s1 Mus musculus cDNA, 3 end/clone = UI-M-AI0-aat-b-06-0-UI/clone_end = 3/ | 1245.5 | P | 1597.2 | P | 1409.1 | P |
| | | gb = AI835846/gi = 5470059/ug = Mm.30068/len = 182/STRA = for | | | | | | |
| 15 | 96942_at | Cluster Incl AI844011: UI-M-AL1-ahi-b-01-0-UI s1 Mus musculus cDNA, 3 end/clone = UI-M-AL1-ahi-b-01-0-UI/ | 2857.2 | P | 2822.9 | P | 3034 | P |
| | | clone_end = 3/gb = AI844011/gi = 5487917/ug = Mm 30194/len = 338/STRA = for | | | | | | |
| 15 | 96949_at | Cluster Incl AI853542 UI-M-BH0-ajd-g-01-0-UI s1 Mus musculus cDNA, 3 end/clone = UI-M-BH0-ajd-g-01-0-UI/ | 1041.2 | P | 814.1 | P | 969.9 | P |
| | | clone_end = 3/gb = AI853542/gi = 5497448/ug = Mm 30205/len = 110/STRA = for | | | | | | |
| 15 | 97460_at | Cluster Incl AW122823: UI-M-BH2 1-aoz-c-09-0-UI.s1 Mus musculus cDNA, 3 end/clone = UI-M-BH2 1-aoz-c-09-0-UI/ | 1783.5 | P | 1756.5 | P | 1915.1 | P |
| | | clone_end = 3/gb = AW122823/gi = 6098353/ug = Mm 30272/len = 219/STRA = for | | | | | | |
| 15 | 97484_at | Cluster Incl AI852704: UI-M-BH0-aji-e-07-0-UI s1 Mus musculus cDNA, 3 end/clone = UI-M-BH0-aji-e-07-0-UI/clone_end = 3/ | 775.9 | P | 1002.7 | P | 1177.8 | P |
| | | gb = AI852704/gi = 5496610/ug = Mm.30826/len = 427/STRA = for | | | | | | |
| 15 | 97544_at | Cluster Incl D83037 Mouse mRNA for 14-3-3 zeta, complete cds/cds = (87,824)/gb = D83037/gi = 1181605/ug = Mm 3360/ | 2055.4 | P | 2305.4 | P | 2482.5 | P |
| | | len = 1935/STRA = for | | | | | | |
| 15 | 97553_at | Cluster Incl AI841884 UI-M-AO0-acd-c-10-0-UI.s1 Mus musculus cDNA, 3 end/clone = UI-M-AO0-acd-c-10-0-UI/ | 476 | P | 483 | P | 449.1 | P |
| | | clone_end = 3/gb = AI841884/gi = 5476097/ug = Mm 33808/len = 370/STRA = for | | | | | | |
| 15 | 98059_s_at | Cluster Incl D49733: Lamin A/cds = (0,1388)/gb = D49733/gi = 1838920/ug = Mm.3438/len = 1480/STRA = for | 5393.9 | P | 5321.9 | P | 5580.8 | P |
| 15 | 98078_at | Cluster Incl AI157911 ue57h08 r1 Mus musculus cDNA, 5 end/clone = IMAGE-1495263/clone_end = 5/gb = AI157911/ | 286.9 | P | 389.5 | P | 377 | P |
| | | gi = 3686380/ug = Mm 34556/len = 439/STRA = for | | | | | | |
| 15 | 98094_f_at | Cluster Incl AI843627 UI-M-AO1-aen-f-03-0-UI.s1 Mus musculus cDNA, 3 end/clone = UI-M-AO1-aen-f-03-0-UI/ | 587.4 | P | 642.5 | P | 597.2 | P |
| | | clone_end = 3/gb = AI843627/gi = 5477840/ug = Mm.34641/len = 425/STRA = for | | | | | | |
| 15 | 98119_at | Cluster Incl K02928 Ribosomal protein L30/cds = (70,417)/gb = K02928/gi = 435126/ug = Mm 3487/len = 468/STRA = for | 5073 | P | 5611.4 | P | 5520.5 | P |
| 15 | 98152_at | Cluster Incl AW122407 UI-M-BH2 2-aow-e-09-0-UI.s1 Mus musculus cDNA, 3 end/clone = UI-M-BH2.2-aow-e-09-0-UI/ | 1294.7 | P | 1198.5 | P | 1490.6 | P |
| | | clone_end = 3/gb = AW122407/gi = 6097909/ug = Mm 35738/len = 471/STRA = for | | | | | | |
| 15 | 98154_at | Cluster Incl AW050133 UI-M-BH1-ano-d-11-0-UI s1 Mus musculus cDNA, 3 end/clone = UI-M-BH1-ano-d-11-0-UI/ | 465.1 | P | 437.4 | P | 458.1 | P |
| | | clone_end = 3/gb = AW050133/gi = 5910662/ug = Mm 35779/len = 521/STRA = for | | | | | | |
| 15 | 98557_f_at | Cluster Incl U65636 Mus musculus beta proteasome subunit (Lmp3) mRNA, complete cds/cds = (5,799)/gb = U65636/ | 2351.4 | P | 2137.3 | P | 2502.6 | P |
| | | gi = 1762778/ug = Mm 368/len = 892/STRA = for | | | | | | |
| 15 | 98564_f_at | Cluster Incl U67770 Mus musculus ribosomal protein S26 (RPS26) mRNA, complete cds/cds(5,352)/gb = U67770/ | 3160.5 | P | 3704.4 | P | 4042.3 | P |
| | | gi = 1527175/ug = Mm 372/len = 426/STRA = for | | | | | | |
| 15 | 98571_s_at | Cluster Incl U48363 Nascent polypeptide-associated complex alpha polypeptide/cds = (51,698)/gb = U48363/gi = 1686688/ | 4341.1 | P | 4336.8 | P | 4414.1 | P |
| | | ug = Mm 3746/len = 829/STRA = for | | | | | | |
| 15 | 99093_at | Cluster Incl AI852864: UI-M-BH0-ajk-e-04-0-UI s1 Mus musculus cDNA, 3 end/clone = UI-M-BH0-ajk-e-04-0-UI/ | 5628.2 | P | 6095.3 | P | 6751 | P |
| | | clone_end = 3/gb = AI852864/gi = 5496770/ug = Mm 39130/len = 308/STRA = for | | | | | | |
| 15 | 99096_at | Cluster Incl U46690 Mus musculus ATP-dependent RNA helicase mRNA, partial cds/cds = (0,1441)/gb = U46690/ | 229.9 | P | 340.4 | P | 324.7 | P |
| | | gi = 1262844/ug = Mm 3935/len = 1674/STRA = for | | | | | | |
| 15 | 99120_f_at | Cluster Incl R75450 MDB0625 Mus musculus cDNA, 3 end/clone_end = 3/gb = R75450/gi = 849694/ug = Mm.4024/ | 1822.3 | P | 2636.6 | P | 2480.5 | P |
| | | len = 329/STRA = for | | | | | | |
| 15 | 99126_at | Cluster Incl L04961 Inactive X specific transcripts/cds = UNKNOWN/gb = L04961/gi = 202420/ug = Mm 4095/len = 14739/ | 4391.8 | P | 172 | P | 3991.3 | P |
| | | STRA = for | | | | | | |
| 15 | 99135_at | Cluster Incl U43076 Mus musculus cdc37 homolog mRNA, complele cds/cds = (87,1226)/gb = U43078/gi = 1375488/ | 882.1 | P | 839.6 | P | 1175.6 | P |
| | | ug = Mm 4117/len = 1607/STRA = for | | | | | | |
| 15 | 99586_at | Cluster Incl U10098 Cystalin 3/cds = (0,422)/gb = U10098/gi = 497414/ug = Mm 4263/len = 423/STRA = for | 4860.7 | P | 3351.3 | P | 4547 | P |
| 15 | 99590_at | Cluster Incl D25213 Mouse rpS17 mRNA for ribosomal protein S17, complete cds/cds = (11,418)/gb = D25213/gi = 893394/ | 3721.4 | P | 3941 | P | 4249.9 | P |
| | | ug = Mm 42767/len = 453/STRA = for | | | | | | |
| 15 | 99592_f_at | Cluster Incl AB030505 Mus musculus mRNA for UBE-1c1, UBE-1c2, UBE-1c3, complete cds/cds = UNKNOWN/ | 1110.9 | P | 1124.5 | P | 1021.1 | P |
| | | gb = AB030505/gi = 5668736/ug = Mm.42795/len = 4190/STRA = for | | | | | | |
| 15 | 99640_at | Cluster Incl AW045481: UI-M-BH1-ahr-c-09-0-UI s1 Mus musculus cDNA, 3 end/clone = UI-M-BH1-ai.r-c-09-0-UI/ | 652.2 | P | 809.8 | P | 762 | P |
| | | clone_end = 3/gb = AW045481/gi = 5905975/ug = Mm 43580/len = 398/STRA = for | | | | | | |
| 15 | 99660_f_at | Cluster Incl X52940: Cytochrome C oxidase subunit VIIc/cds = (86,277)/gb = X52940/gi = 50524/ug = Mm 43786/len = 403/ | 4434.3 | P | 5200 | P | 5262.8 | P |
| | | STRA = for | | | | | | |
| 15 | 99661_r_at | Cluster Incl X52940: Cytochrome C oxidase subunit VIIc/cds = (86,277)/gb = X52940/gi = 50524/ug = Mm.43786/len = 403/ | 1814.6 | P | 1891.2 | P | 2459.8 | P |
| | | STRA = for | | | | | | |
| 15 | 99669_at | Cluster Incl X15986: Mouse 3 mRNA for beta-galactoside specific lectin (14 kDa)/cds = (43,450)/gb = X15986/gi = 50162/ | 5510.3 | P | 3817.5 | P | 5245.4 | P |
| | | ug = Mm 43831/len = 495/STRA = for | | | | | | |
| 15 | 100043_f_at | Cluster Incl AI173973: ui43c05 x1 Mus musculus cDNA, 3 end/clone = IMAGE-1885160/clone_end = 3/gb = AI173973/ | 3441.9 | P | 2789.7 | P | 3566.9 | P |
| | | gi = 3720115/ug = Mm 44242/len = 459/STRA = for | | | | | | |
| 15 | 100064_f_at | Cluster Incl M63801 Mus musculus connexin 43 (alpha-1 gap junction) mRNA, complete cds/cds = (220,1388)/gb = M63801/ | 4721.7 | P | 4910.8 | P | 5045.8 | P |
| | | gi = 191773/ug = Mm 4504/len = 1408/STRA = for | | | | | | |
| 15 | 100569_at | Cluster Incl M14044 Mouse calpactin I heavy chain (p36) mRNA, complete cds/cds = (7,1026)/gb = M14044/gi = 192353/ | 3853.1 | P | 4129.5 | P | 4615.5 | P |
| | | ug = Mm 584/len = 1307/STRA = for | | | | | | |
| 15 | 100618_f_at | Cluster Incl AA062013 mj83h01 r1 Mus musculus cDNA, 5 end/clone = IMAGE-482737/clone_end = 5/gb = AA062013/ | 1546.6 | P | 1578.8 | P | 1683.8 | P |
| | | gi = 1555829/ug = Mm.658/len = 529/STRA = for | | | | | | |
| 15 | 101093_at | Cluster Incl M15832: Procollagen, type IV, alpha 1/cds = (0,689)/gb = M15832/gi = 192282/ug = Mm 738/len = 690/STRA = for | 3890.5 | P | 4452.5 | P | 4873.3 | P |
| 15 | 101113_at | Cluster Incl AI846668 UI-M-AN1-afi-c-10-0-UI s1 Mus musculus cDNA, 3 end/clone = UI-M-AN1-afi-c-10-0-UI/clone_end = 3/ | 1463.9 | P | 1301.2 | P | 1769 | P |
| | | gb = AI846668/gi = 5490574/ug = Mm.757/len = 262/STRA = for | | | | | | |
| 15 | 101542_f_at | Cluster Incl L25126 D-E-A-D (aspartate-glutamahe-alanine-aspartate) box polypeptide 3/cds = (23,2011)/gb = L25126/ | 2591 | P | 2372.3 | P | 2769.4 | P |
| | | gi = 407995/ug = Mm 88188/len = 2187/STRA = for | | | | | | |
| 15 | 101543_f_at | Cluster Incl M13441: Mouse alpha-tubulin isotype M-alpha-6 mRNA, complete cds/cds = (43,1392)/gb = M13441/gi = 202216/ | 2742.2 | P | 3557.2 | P | 4038 | P |
| | | ug = Mm.88212/len = 1495/STRA = for | | | | | | |
| 15 | 101573_f_at | Cluster Incl AB020237: Mus musculus gene for ribosomal protein L27A, complete cds/cds = (0,446)/gb = AB020237/ | 4957.3 | P | 5040.6 | P | 5286.4 | P |
| | | gi = 4760603/ug = Mm 89853/len = 447/STRA = for | | | | | | |
| 15 | 101577_at | Cluster Incl Z54209 M. musculus rpS6 gene/cds = (41,790)/gb = Z54209/gi = 1177548/ug = Mm.89902/len = 831/STRA = for | 4358.6 | P | 4684.9 | P | 5096.1 | P |
| 15 | 101578_f_at | Cluster Incl M12481 Actin, beta, cytoplasmic/cds = (0,1049)/gb = M12481/gi = 191581/ug = Mm.89919/len = 1109/STRA = for | 4312.2 | P | 4331.8 | P | 4212.3 | P |
| 15 | 102000_f_at | Cluster Incl AI842835 UI-M-AN1-afb-a-07-0-UI s1 Mus musculus cDNA, 3 end/clone = UI-M-AN1-afb-a-07-0-UI/ | 1010.8 | P | 1087.2 | P | 1207.3 | P |
| | | clone_end = 3/gb = AI842835/gi = 5477048/ug = Mm 988/len = 287/STRA = for | | | | | | |
| 15 | 102028_at | Cluster Incl AF053959 Mus musculus putative ras effector Nore1 mRNA, complete cds/cds = (30,1271)/gb = AF053959/ | 430.3 | P | 549.3 | P | 566.7 | P |
| | | gi = 2997697/ug = Mm.10133/len = 3018/STRA = for | | | | | | |
| 15 | 102364_at | Cluster Incl J04509 Jun proto-oncogene related gene d1/cds = (121,1146)/gb = J04509/gi = 198486/ug = Mm.1175/len = 1147/ | 2806.7 | P | 3583 | P | 3607.2 | P |
| | | STRA = for | | | | | | |
| 15 | 102414_i_at | Cluster Incl U28423 Mus musculus protein kinase inhibitor p58 mRNA, complete cds/cds = (100,1614)/gb = U28423/ | 246 | P | 457 | P | 479.2 | P |
| | | gi = 1353271/ug = Mm.12616/len = 1615/STRA = for | | | | | | |
| 15 | 102856_at | Cluster Incl AF047389 SRY-box containing gene 10/cds = (53,931)/gb = AF047389/gi = 3264587/ug = Mm 1493/len = 1463/ | 584.2 | P | 597.7 | P | 509.3 | P |
| | | STRA = for | | | | | | |
| 15 | 102986_at | Cluster Incl M18779 Myogenic differentiation 1/cds = (159,1115)/gb = M18779/gi = 199994/ug = Mm 1526/len = 1785/ | 310.3 | P | 344.6 | P | 400.9 | P |
| | | STRA = for | | | | | | |
| 15 | 103079_at | Cluster Incl AA982208: vw61e08 s1 Mus musculus cDNA, 3 end/clone = IMAGE-1248326/clone_end = 3/gb = AA982208/ | 909.6 | P | 924.5 | P | 926.1 | P |
| | | gi = 3160867/ug = Mm 17166/len = 415/STRA = for | | | | | | |
| 15 | 103082_at | Cluster Incl AI847507: UI-M-AM1-afv-a-08-0-UI s1 Mus musculus cDNA, 3 end/clone = UI-M-AM1-afv-a-08-0-UI/ | 683.4 | P | 517.4 | P | 660.8 | P |
| | | clone_end = 3/gb = AI847507/gi = 5491413/ug = Mm 17173/len = 313/STRA = for | | | | | | |
| 15 | 103388_at | Cluster Incl AW047050: UI-M-BH1-alr-c-07-0-UI.s1 Mus musculus cDNA, 3 end/clone = UI-M-BH1-alr-c-07-0-UI/ | 1418 | P | 1262.3 | P | 1397.1 | P |
| | | clone_end = 3/gb = AW047050/gi = 5907579/ug = Mm 1865/len = 318/STRA = rev | | | | | | |
| 15 | 103611_at | Cluster Incl AB012693 Mus musculus mRNA for CD47, complete cds/cds = (72,1046)/gb = AB012693/gi = 3036964/ | 1252.1 | P | 1186.6 | P | 1333.7 | P |
| | | ug = Mm 20183/len = 1250/STRA = rev | | | | | | |
| 15 | 103846_at | Cluster Incl AF038582 Mus musculus galectin-7 mRNA, complete cds/cds = (20,430)/gb = AF038562/gi = 2708653/ | 3469.1 | P | 3672.1 | P | 3714.5 | P |
| | | ug = Mm 20973/len = 588/STRA = for | | | | | | |
| 15 | 103862_r_at | Cluster Incl AA388099: vc88g07 r1 Mus musculus cDNA, 5 end/clone = IMAGE-790140/clone_end = 5/gb = AA388099/ | 1604.7 | P | 1351.1 | P | 1162.9 | P |
| | | gi = 2041044/ug = Mm 21103/len = 564/STRA = for | | | | | | |
| 15 | 103878_at | Cluster Incl AF103809 Mus musculus Ap-3 complex beta3A subunit (Ap3b1) mRNA, complete cds/cds = (63,3380)/ | 885.1 | P | 852.2 | P | 829.9 | P |
| | | gb = AF103809/gi = 3885987/ug = Mm 21185/len = 3938/STRA = for | | | | | | |
| 15 | 103894_at | Cluster Incl AW060871: UI-M-BH1-amx-f-12-0-UI s1 Mus musculus cDNA, 3 end/clone = UI-M-BH1-amx-f-12-0-UI/ | 2672.7 | P | 2378 | P | 2659.1 | P |
| | | clone_end = 3/gb = AW060871/gi = 6008622/ug = Mm 21297/len = 463/STRA = for | | | | | | |
| 15 | 104106_at | Cluster Incl AI837830: UI-M-AP0-abk-a-07-0-UI s1 Mus musculus cDNA, 3 end/clone = UI-M-AP0-abk-a-07-0-UI/ | 246.7 | P | 258.3 | P | 341.7 | P |
| | | clone_end = 3/gb = AI837830/gi = 5472043/ug = Mm 21897/len = 259/STRA = for | | | | | | |
| 15 | 104311_at | Cluster Incl AW125241: UI-M-BH2 1-aps-e-11-0-UI s1 Mus musculus cDNA, 3 end/clone = UI-M-BH2 1-aps-e-11-0-UI/ | 155.3 | P | 187.6 | P | 376.8 | P |
| | | clone_end = 3/gb = AW125241/gi = 6100771/ug = Mm 22397/len = 320/STRA = for | | | | | | |
| 15 | 104313_at | Cluster Incl AI842432 UI-M-AM1-afy-b-10-0-UI s1 Mus musculus cDNA, 3 end/clone = UI-M-AM1-afy-b-10-0-UI/ | 1603.5 | P | 1309.8 | P | 1581.2 | P |
| | | clone_end = 3/gb = AI842432/gi = 5476645/ug = Mm 22403/len = 330/STRA = for | | | | | | |
| 15 | 104341_at | Cluster Incl AW121406 UI-M-BH2 2-aom-h-09-0-UI s1 Mus musculus cDNA, 3 end/clone = UI-M-BH2 2-aom-h-09-0-UI/ | 1328.3 | P | 1426.7 | P | 1308.9 | P |
| | | clone_end = 3/gb = AW121406/gi = 6096739/ug = Mm 22525/len = 498/STRA = for | | | | | | |
| 15 | 104362_at | Cluster Incl AI853532 UI-M-BHO-ajd-f-02-0-UI s1 Mus musculus cDNA, 3 end/clone = UI-M-BH0-ajd-f-02-0-UI/ | 421.7 | P | 339.1 | P | 432.6 | P |
| | | clone_end = 3/gb = AI853532/gi = 5497438/ug = Mm.22599/len = 353/STRA = for | | | | | | |
| 15 | 104391_s_at | Cluster Incl AI850563 UI-M-BG1-aij-e-12-0-UI s1 Mus musculus cDNA, 3 end/clone = UI-M-BG1-aij-e-12-0-UI/ | 443.7 | P | 561.2 | P | 543.9 | P |
| | | clone_end = 3/gb = AI850563/gi = 5494469/ug = Mm.22727/len = 271/STRA = for | | | | | | |
| 15 | 104524_at | Cluster Incl AI842825: UI-M-AP1-agi-h-07-0-UI s1 Mus musculus cDNA, 3 end/clone = UI-M-AP1-agi-h-07-0-UI/ | 3877.2 | P | 4165.5 | P | 4490.1 | P |
| | | clone_end = 3/gb = AI842825/gi = 5477038/ug = Mm 22986/len = 341/STRA = for | | | | | | |
| 15 | 104559_at | Cluster Incl AI848162 UI-M-AH1-agq-c-02-0-UI s1 Mus musculus cDNA, 3 end/clone = UI-M-AH1-asg-c-02-0-UI/ | 1137 | P | 1214.1 | P | 1421.3 | P |
| | | clone_end = 3/gb = AI848162/gi = 5492068/ug = Mm 23826/len = 492/STRA = for | | | | | | |
| 15 | 104576_at | Cluster Incl AW212397: uo86e12.x1 Mus musculus cDNA, 3 end/clone = IMAGE-2649454/clone_end = 3/gb = AW212397/ | 1187.7 | P | 1171.6 | P | 1193.4 | P |
| | | gi = 6518452/ug = Mm 23941/len = 165/STRA = for | | | | | | |
| 15 | 104710_at | Cluster Incl Y13231: Bcl2 homologous antagonist/killer/cds = (254,880)/gb = Y13231/gi = 2113878/ug = Mm 2443/len = 2005/ | 780.5 | P | 609.6 | P | 815.2 | P |
| | | STRA = for | | | | | | |
| 15 | 94341_at | Cluster Incl D31967 Jumonji/cds = (210,3914)/gb = D31967/gi = 780143/ug = Mm.25059/len = 4071/STRA = for | 1453.4 | P | 1719.6 | P | 1627.9 | P |
| 15 | 94929_at | Cluster Incl M97590: Mouse tyrosine phosphatase (PTP-1), (EC2 7 1.112) mRNA/cds = UNKNOWN/gb = M97590/ | 996.9 | P | 822.2 | P | 1016 | P |
| | | gi = 200581/ug = Mm 2668/len = 1664/STRA = for | | | | | | |
| 15 | 94972_at | Cluster Incl AB026569 Mus musculus mRNA for MSSP, complete cds/cds = (261,1373)/gb = AB026569/gi = 4730905/ | 586.3 | P | 825 | P | 681.3 | P |
| | | ug = Mm 27230/len = 1650/STRA = for | | | | | | |
| 15 | 95627_at | Cluster Incl AW046038 UI-M-BH1-akv-b-03-0-U1.s1 Mus musculus cDNA, 3 end/clone = UI-M-BH1-akv-b-03-0-UI/ | 1124 | P | 715 | P | 776.3 | P |
| | | clone_end = 3/gb = AW046038/gi = 5906567/ug = Mm 28060/len = 433/STRA = for | | | | | | |
| 15 | 96197_f_at | Cluster Incl AI851708 UI-M-BHO-aio-d-08-0-UI s1 Mus musculus cDNA, 3 end/clone = UI-M-BH0-aio-d-08-0-UI/ | 397.8 | P | 389.1 | P | 364.8 | P |
| | | clone_end = 3/gb = AI851708/gi = 5495609/ug = Mm.28553/len = 337/STRA = for | | | | | | |
| 15 | 96220_at | Cluster Incl AW123157 UI-M-BH2 1-apd-c-10-0-UI.s1 Mus musculus cDNA, 3 end/clone = UI-M-BH2 1-apd-c-10-0-UI/ | 420.7 | P | 343.9 | P | 324.1 | P |
| | | clone_end = 3/gb = AW123157/gi = 6098687/ug = Mm 28709/len = 542/STRA = for | | | | | | |
| 15 | 97375_at | Cluster Incl U70209 Mus musculus polycystic kidney disease 1 protein (Pkd1) mRNA, complete cds/cds = (315,13196)/ | 192 | P | 173.6 | P | 178 | P |
| | | gb = U70209/gi = 2138182/ug = Mm 2973/len = 14141/STRA = for | | | | | | |
| 15 | 97426_at | Cluster Incl X98471: Epithelial membrane protein 1/cds = (0,482)/gb = X98471/gi = 1580765/ug = Mm.30024/len = 483/ | 3549.6 | P | 3302.7 | P | 3306 | P |
| | | STRA = for | | | | | | |
| 15 | 97937_at | Cluster Incl AA611766: vo85108.r1 Mus musculus cDNA, 5 end/clone = IMAGE-1065927/clone_end = 5/gb = AA611766/ | 1161.5 | P | 1236.1 | P | 1831.9 | P |
| | | gi = 2461801/ug = Mm.30262/len = 161/STRA = rev | | | | | | |
| 15 | 98432_at | Cluster Incl X99384: M musculus mRNA for paladin gene/cds = (46,2625)/gb = X99384/gi = 1545958/ug = Mm.3437/len = 4055/ | 205 | P | 147.5 | P | 177.6 | P |
| | | STRA = for | | | | | | |
| 15 | 98436_s_at | Cluster Incl U54803 Caspase 3, apoptosis related cysteine protease/cds = (15,848)/gb = U54803/gi = 1518967/ | 265.7 | P | 256.9 | P | 262.7 | P |
| | | ug = Mm 34405/len = 1418/STRA = rev | | | | | | |
| 15 | 98477_s_at | Cluster Incl L40632 Ankyrin 3, epithelial/cds = (56,5941)/gb = L40632/gi = 710548/ug = Mm 3526/len = 7084/STRA = for | 164.1 | P | 145.6 | P | 185.8 | P |
| 15 | 98984_I _at | Cluster Incl D50430 Mouse mRNA for glycerol-3-phosphate dehydrogenase, complete cds/cds = (96,2279)/gb = D50430/ | 285.1 | P | 377.6 | P | 320.3 | P |
| | | gi = 1339937/ug = Mm 3711/len = 2410/STRA = for | | | | | | |
| 15 | 99054_at | Cluster Incl D16141: Lethal giant larvae homolog/cds = (21,3125)/gb = D16141/gi = 414350/ug = Mm 3940/len = 4279/ | 1129.4 | P | 1023.7 | P | 1185.2 | P |
| | | STRA = rev | | | | | | |
| 15 | 99458_I_at | Cluster Incl X70764 ELKL motif kinase/cds = (93,2417)/gb = X70764/gi = 57919/ug = Mm 4082/len = 2763/STRA = for | 2225.2 | P | 2540.6 | P | 2229.8 | P |
| 15 | 99934_at | Cluster Incl M80206 Poliovirus sensitivity/cds = (0,1403)/gb = M80206/gi = 199785/ug = Mm 4341/len = 1404/STRA = for | 612.9 | P | 641.5 | P | 653.7 | P |
| 15 | 100011_at | Cluster Incl AI851658: UI-M-BH0-ain-f-03-0-UI s1 Mus musculus cDNA, 3 end/clone = UI-M-BH0-ain-f-03-0-UI/ | 2213.2 | P | 1827.3 | P | 1717 | P |
| | | clone_end = 3/gb = AI851658/gi = 5495564/ug = Mm 4549/len = 448/STRA = for | | | | | | |
| 15 | 100027_s_at | Cluster Incl AI881987 uI56h05.x1 Mus musculus cDNA, 3 end/clone = IMAGE-2123097/clone_end = 3/gb = AI881987/ | 718.3 | P | 652 | P | 684.4 | P |
| | | gi = 5567139/ug = Mm 4609/len = 512/STRA = for | | | | | | |
| 15 | 100029_at | Cluster Incl AW208667: uo62e02 x1 Mus musculus cDNA, 3 end/clone = IMAGE-2647130/clone_end = 3/gb = AW208667/ | 2179.2 | P | 2137 | P | 1974.2 | P |
| | | gi = 6514607/ug = Mm 4609/len = 474/STRA = for | | | | | | |
| 15 | 100032_at | Cluster Incl X60136 Trans-acting transcription factor 1/cds = (0,307)/gb = X60136/gi = 54158/ug = Mm 4618/len = 454/ | 870.3 | P | 721.8 | P | 734.6 | P |
| | | STRA = for | | | | | | |
| 15 | 100401_at | Cluster Incl AA673981: vo87c03 r1 Mus musculus cDNA, 5 end/clone = IMAGE-1066084/clone_end = 5/gb = AA673981/ | 611 | P | 612.4 | P | 583 | P |
| | | gi = 2647263/ug = Mm 46401/len = 358/STRA = for | | | | | | |
| 15 | 100420_at | Cluster Incl J03458 Filaggnn/cds = (0,1010)/gb = J03458/gi = 193307/ug = Mm 4685/len = 1488/STRA = for | 4702 | P | 5169.2 | P | 4617.5 | P |
| 15 | 100514_at | Cluster Incl M63660 Mouse G-alpha-13 protein mRNA, complete cds/cds = (128,1261)/gb = M63660/gi = 193378/ | 915.7 | P | 858.6 | P | 889.7 | P |
| | | ug = Mm 5238/len = 2422/STRA = rev | | | | | | |
| 15 | 101406_at | Cluster Incl AF026072 Mus musculus hydroxysteroid sulfotransferase (SULT2B) mRNA, complete cds/cds = (69,1085)/ | 650.3 | P | 618.5 | P | 794 | P |
| | | gb = AF026072/gi = 2570897/ug = Mm 7316/len = 1127/STRA = for | | | | | | |
| 15 | 101448_at | Cluster Incl D50050: HGF-regulated tyrosine kinase substrate/cds = (42,2369)/gb = D50050/gi = 1089780/ug = Mm.7919/ | 2130.7 | P | 2142.4 | P | 1884.6 | P |
| | | len = 2458/STRA = for | | | | | | |
| 15 | 101922_at | Cluster Incl AW123408 UI-M-BH2 1-apj-b-01-0-UI.s1 Mus musculus cDNA, 3 end/clone = UI-M-BH2.1-apj-b-01-0-UI/ | 671.6 | P | 668.2 | P | 830.5 | P |
| | | clone_end = 3/gb = AW123408/gi = 6098938/ug = Mm 92288/len = 410/STRA = for | | | | | | |
| 15 | 102222_at | Cluster Incl AJ002730 Ubiquitously transcribed tetratricopeptide repeat gene, X Chromosome/cds = (0,4003)/gb = AJ002730/ | 681.1 | P | 332.4 | P | 834.3 | P |
| | | gi = 3021456/ug = Mm 10218/len = 4215/STRA = rev | | | | | | |
| 15 | 102273_at | Cluster Incl M31226 Mouse immune suppressor factor TJ6 mRNA, complete cds/cds = (18,2585)/gb = M31226/gi = 198460/ | 336.1 | P | 323.9 | P | 378.8 | P |
| | | ug = Mm 1158/len = 2936/STRA = for | | | | | | |
| 15 | 102691_at | Cluster Incl AI848518 UI-M-AP1-agf-e-02-0-UI s2 Mus musculus cDNA, 3 end/clone = UI-M-AP1-agf-e-02-0-UI/ | 721.3 | P | 891 | P | 1007.2 | P |
| | | clone _end _3/gb = AI848518/gi = 5492424/ug = Mm.14099/len = 361/STRA = for | | | | | | |
| 15 | 102727_at | Cluster Incl X55573 Brain derived neurotrophic factor/cds = (77,826)/gb = X55573/gi = 287898/ug = Mm 1442/len = 927/ | 337.2 | P | 294.2 | P | 316 | P |
| | | STRA = for | | | | | | |
| 15 | 103269_f_at | Cluster Incl AJ005350 Mus musculus of ZT2 gene encoding zinc finger protein 125/cds = (0,541)/gb = AJ005350/ | 216.1 | P | 306.8 | P | 244.5 | P |
| | | gi = 3646358/ug = Mm.20857/len = 2440/STRA = for | | | | | | |
| 15 | 103288_at | Cluster Incl AF053062 Mus musculus receptor interacting protein 140 mRNA, complete cds/cds = (371,3856)/ | 664.5 | P | 458 | P | 474.9 | P |
| | | gb = AF053062/gi = 3820493/ug = Mm 20895/len = 4307/STRA = for | | | | | | |
| 15 | 103484_at | Cluster Incl AA619442 vo84c10.r1 Mus musculus cDNA, 5 end/clone = IMAGE-1065810/clone_end = 5/gb = AA619442/ | 50.8 | P | 43.2 | P | 46.7 | P |
| | | gi = 2523318/ug = Mm.22133/len = 464/STRA = for | | | | | | |
| 15 | 103709_at | Cluster Incl AA763466 vw54f05 r1 Mus musculus cDNA, 5 end/clone = IMAGE-1247649/clone_end = 5/gb = AA763466/ | 3031 | P | 3269.3 | P | 3362.5 | P |
| | | gi = 2813213/ug = Mm 24093/len = 379/STRA = for | | | | | | |
| 15 | 103771_at | Cluster Incl AI837993 UI-M-AP0-abl-d-01-0-UI s1 Mus musculus cDNA, 3 end/clone = UI-M-AP0-abl-d-01-0-UI/ | 1365.1 | P | 1502.8 | P | 1630.2 | P |
| | | clone_end = 3/gb = AI837993/gi = 54722067/ug = Mm 24777/len = 336/STRA = rev | | | | | | |
| 15 | 103774_at | Cluster Incl AW108350: um29f06 x1 Mus musculus cDNA, 3 end/clone = IMAGE-2235971/clone_end = 3/gb = AW108350/ | 303.1 | P | 482 | P | 475.2 | P |
| | | gi = 6079150/ug = Mm 24790/len = 579/STRA = for | | | | | | |
| 15 | 103978_at | Cluster Incl AA666669: vm50f06.r1 Mus musculus cDNA, 5 end/clone = IMAGE-1001699/clone_end = 5/gb = AA666669/ | 789.6 | P | 826.2 | P | 857.8 | P |
| | | gi = 2625370/ug = Mm 25787/len = 412/STRA = for | | | | | | |
| 15 | 103994_at | Cluster Incl AI152867: ud93h11.r1 Mus musculus cDNA, 5 end/clone = IMAGE-1478469/clone_end = 5/gb = AI152867/ | 1339.2 | P | 866.3 | P | 1394.9 | P |
| | | gi = 3681336/ug = Mm 26266/len = 471/STRA = for | | | | | | |
| 15 | 104158_at | Cluster Incl AW046671: UI-M-BH1-alm-a-09-0-UI s1 Mus musculus cDNA, 3 end/clone = UI-M-BH1-alm-a-09-0-UI/ | 217.2 | P | 186.3 | P | 294.6 | P |
| | | clone_end = 3/gb = AW046671/gi = 5907200/ug = Mm.27094/len = 240/STRA = for | | | | | | |
| 15 | 104225_at | Cluster Incl AI645050 vs46d05 y1 Mus musculus cDNA, 5 end/clone = IMAGE-1149321/clone_end = 5/gb = AI645050/ | 411.4 | P | 492.3 | P | 504 | P |
| | | gi = 4723525/ug = Mm 27988/len = 373/STRA = rev | | | | | | |
| 15 | 104407_at | Cluster Incl L25274 Mus musculus transmembrane glycoprotein (DM-GRASP) mRNA, 3 end/cds = (0,1075)/gb = L25274/ | 324.6 | P | 474.9 | P | 326.4 | P |
| | | gi = 452102/ug = Mm 2877/len = 1288/STRA = for | | | | | | |
| 15 | 104433_at | Cluster Incl AI849682 UI-M-AL1-ahl-f-01-0-UI s1 Mus musculus cDNA, 3 end/clone = UI-M-AL1-ahl-f-01-0-UI/clone_end = 3/ | 383.5 | P | 506.5 | P | 480.5 | P |
| | | gb = AI849682/gi = 5493588/ug = Mm 29228/len = 245/STRA = rev | | | | | | |
| 15 | 104461_at | Cluster Incl AW121773 UI-M-BH2 3-aoe-f-11-0-UI s1 Mus musculus cDNA, 3 end/clone = UI-M-BH2.3-aoe-f-11-0-UI/ | 180.6 | P | 279.1 | P | 254.2 | P |
| | | clone_end = 3/gb = AW121773/gi = 6097121/ug = Mm 29589/len = 166/STRA = for | | | | | | |
| 15 | 104663_at | Cluster Incl D86177 Mouse mRNA for phosphatidylinositol 4-phosphate 5-kinase type l-beta, complete cds/cds = (269,1909)/ | 511.4 | P | 453.9 | P | 683.1 | P |
| | | gb = D86177/gi = 1622709/ug = Mm 3191/len = 2403/STRA = for | | | | | | |
| 15 | 104682_at | Cluster Incl AI848983: UI-M-AJ1-ahd-d-05-0-UI s1 Mus musculus cDNA, 3 end/clone = UI-M-AJ1-ahd-d-05-0-UI/ | 848.1 | P | 922.2 | P | 1115.1 | P |
| | | clone_end = 3/gb = AI848983/gi = 5492889/ug = Mm.32884/len = 499/STRA = for | | | | | | |
| 15 | 92195_at | Cluster Incl AB012273 Mus musculus mRNA for GPE1-BP (C/EBP gamma), complete cds/cds = (224,676)/gb = AB012273/ | 278.3 | P | 347.5 | P | 347.4 | P |
| | | gi = 2988367/ug = Mm 3402/len = 1185/STRA = for | | | | | | |
| 15 | 92444_f_at | Cluster Incl X16493: Zinc finger protein 1/cds = (320,1594)/gb = X16493/gi = 55482/ug = Mm.4184/len = 1939/STRA = rev | 266.7 | P | 274.1 | P | 275 | P |
| 15 | 92452_at | Cluster Incl U03279: Phosphatidylinositol 3-kinase, catalytic, alpha polypeptide/cds = (0,3206)/gb = U03279/gi = 414994/ | 196.3 | P | 228.7 | P | 267.1 | P |
| | | ug = Mm 41943/len = 3207/STRA = rev | | | | | | |
| 15 | 92703_at | Cluster Incl AI325791 mn19e09.y1 Mus musculus cDNA, 5 end/clone = IMAGE-538408/clone_end = 5/gb = AI325791/ | 287.1 | P | 311.6 | P | 375.7 | P |
| | | gi = 4060220/ug = Mm 45992/len = 434/STRA = for | | | | | | |
| 15 | 92891_f_at | Cluster Incl X55318 Homeo box C9/cds = (348,1130)/gb = X55318/gi = 57878/ug = Mm 4765/len = 1434/STRA = for | 116.1 | P | 182.5 | P | 193.9 | P |
| 15 | 92903_at | Cluster Incl X78197 Transcription factor AP-2 beta/cds = (213,1559)/gb = X78197/gi = 496638/ug = Mm 4795/len = 1731/ | 515.2 | P | 328.1 | P | 425.4 | P |
| | | STRA = for | | | | | | |
| 15 | 92918_at | Cluster Incl U66079 Mus musculus coagulation factor VII gene, complete cds/cds = (10,1350)/gb = U66079/gi = 1513221/ | 178.3 | P | 183.6 | P | 158.3 | P |
| | | ug = Mm.4827/len = 1843/STRA = for | | | | | | |
| 15 | 93378_at | Cluster Incl X07439 Mouse mRNA for Hox-3 1 homeo gene/cds = (241,969)/gb = X07439/gi = 51406/ug = Mm 6167/ | 710.4 | P | 838.6 | P | 696.3 | P |
| | | len = 1470/STRA = for | | | | | | |
| 15 | 93478_at | Cluster Incl AA612483: vo72g09.r1 Mus musculus cDNA, 5 end/clone = IMAGE-1064704/clone_end = 5/gb = AA612483/ | 237.3 | P | 277.2 | P | 268.9 | P |
| | | gi = 2461161/ug = Mm.7109/len = 384/STRA = rev | | | | | | |
| 15 | 93938_at | Cluster Incl U17132: Zinc transporter 1/cds = (147,1658)/gb = U17132/gi = 577840/ug = Mm.9024/len = 1720/STRA = for | 427 | P | 582.9 | P | 445.8 | P |
| 15 | 94127_at | Cluster Incl Y15910 Mus musculus mRNA for dia protein/cds = (0,1049)/gb = Y15910/gi = 3171941/ug = Mm.10763/len = 1330/ | 121.6 | P | 188.5 | P | 171.9 | P |
| | | STRA = for | | | | | | |
| 15 | 94127_at | Cluster Incl Y15910 Mus musculus mRNA for dia protein/cds = (0,1049)/gb = Y15910/gi = 3171941/ug = Mm.10763/len = 1330/ | 121.6 | P | 188.5 | P | 171.9 | P |
| | | STRA = for | | | | | | |
| 15 | 94689_at | Cluster Incl C79248 C79248 Mus musculus cDNA, 3 end/clone = J0063C04/clone_end = 3/gb = C79248/gi = 2519578/ | 1316.1 | P | 889.2 | P | 754.1 | P |
| | | ug = Mm 13291/len = 476/STRA = rev | | | | | | |
| 15 | 94767_at | Cluster Incl AB028894 Mus musculus Rps11, U35 genes for ribosomal protein S11 and U35 snoRNA, complete cds and | 4045.2 | P | 4806.5 | P | 5030.4 | P |
| | | sequence/cds = (22,498)/gb = AB028894/gi = 6552367/ug = Mm 164/len = 551/STRA = for | | | | | | |
| 15 | 95305_at | Cluster Incl AA756275 vv40d02 r1 Mus musculus cDNA, 5 end/clone = IMAGE-1224867/clone_end = 5/gb = AA756275/ | 343.1 | P | 321.4 | P | 369.4 | P |
| | | gi = 2803473/ug = Mm 20012/len = 526/STRA = for | | | | | | |
| 15 | 95359_at | Cluster Incl M18186 Mouse 84 kD heat shock protein mRNA, complete cds/cds = (94,2268)/gb = M18186/gi = 194024/ | 1979.7 | P | 2755.4 | P | 3064.5 | P |
| | | ug = Mm 2180/len = 2509/STRA = for | | | | | | |
| 15 | 95385_at | Cluster Incl AI643119 mm55a03 y1 Mus musculus cDNA, 5 end/clone = IMAGE-532300/clone_end = 5/gb = AI643119/ | 458.6 | P | 473.6 | P | 421.9 | P |
| | | gi = 4721594/ug = Mm 23210/len = 285/STRA = rev | | | | | | |
| 15 | 95393_at | Cluster Incl AI503362: vn86g08.x1 Mus musculus cDNA, 3 end/clone = IMAGE-1038878/clone_end = 3/gb = AI503362/ | 280.7 | P | 415.8 | P | 511.8 | P |
| | | gi = 4401213/ug = Mm 23817/len = 425/STRA = for | | | | | | |
| 15 | 95944_at | Cluster Incl AV299153 AV299153 Mus musculus cDNA, 3 end/clone = 5730466C10/clone_end = 3/gb = AV299153/ | 475.5 | P | 471.4 | P | 447.2 | P |
| | | gi = 6331610/ug = Mm 24891/len = 273/STRA = rev | | | | | | |
| 15 | 96522_at | Cluster Incl M19381 Calmodulin/cds = (186,635)/gb = M19381/gi = 469421/ug = Mm.2648/len = 798/STRA = for | 3867.4 | P | 4164.8 | P | 4027.5 | P |
| 15 | 96532_at | Cluster Incl AA212897: mw62a09 r1 Mus musculus cDNA, 5 end/clone = lMAGE-677176/clone_end = 5/gb = AA212897/ | 218.1 | P | 182.3 | P | 173.1 | P |
| | | gi = 1811645/ug = Mm 27297/len = 429/STRA = for | | | | | | |
| 15 | 96542_at | Cluster Incl M62606 Surfeit gene 4/cds = (105,914)/gb = M62606/gi = 201091/ug = Mm 2795/len = 2781/STRA = for | 1921.6 | P | 1916.9 | P | 1950.3 | P |
| 15 | 96573_at | Cluster Incl M21495 Mouse cytoskeletal gamma-actin mRNA, complete cds/cds = (39,1186)/gb = M21495/gi = 191656/ | 6613.1 | P | 6943.4 | P | 7221.2 | P |
| | | ug = Mm 29913/1en = 1868/STRA = for | | | | | | |
| 15 | 96575_at | Cluster Incl U67771: Mus musculus ribosomal protein L8 (RPL8) mRNA, complete cds/cds = (21,794)/gb = U67771/ | 3709.5 | P | 4224.5 | P | 4509.2 | P |
| | | gi = 1527177/ug = Mm 30066/len = 842/STRA = for | | | | | | |
| 15 | 97695_s_at | Cluster Incl M29015.Mouse ribosomal protein L7 (rpL7) gene, complete cds/cds = (0,812)/gb = M29015/gi = 200784/ | 7061.4 | P | 7297.9 | P | 7865.1 | P |
| | | ug = Mm 37835/len = 813/STRA = for | | | | | | |
| 15 | 97696_r_at | Cluster Incl M29015 Mouse ribosomal protein L7 (rpL7) gene, complete cds/cds = (0,812)/gb = M29015/gi = 200784/ | 3265 | P | 3584.5 | P | 4003.5 | P |
| | | ug = Mm 37835/len = 813/STRA = for | | | | | | |
| 15 | 97750_at | Cluster Incl X06406 Mouse mRNA for tranalational controlled 40 kDa polyyeptide p40/cds = (77,964)/gb = X06406/gi = 52852/ | 2394 | P | 2432.5 | P | 2618.1 | P |
| | | ug = Mm 4071/len = 1031/STRA = for | | | | | | |
| 15 | 97751_f_at | Cluster Incl AI835771 UI-M-AI0-aaq-a-05-0-UI s1 Mus musculus cDNA, 3 end/clone = UI-M-AI0-aaq-a-05-0-UI/ | 2058 | P | 2086 | P | 2175.5 | P |
| | | clone_end = 3/gb = AI835771/gi = 5469984/ug = Mm 40749/len = 324/STRA = rev | | | | | | |
| 15 | 98313_at | Cluster InCL AB025413 Mus musculus mRNA for Ten-m4, complete cds/cds = (187,8502)/gb = AB025413/gi = 4760781/ | 215.5 | P | 245.8 | P | 183.3 | P |
| | | ug = Mm 42204/len = 8585/STRA = for | | | | | | |
| 15 | 98319_at | Cluster Incl AJ000328 Mus musculus mRNA tor type 2 desmoglein/cds = (0,245)/gb = AJ000328/gi = 2764500/ | 144.5 | P | 206.6 | P | 223.8 | P |
| | | ug = Mm 42229/len = 745/STRA = for | | | | | | |
| 15 | 98331_at | Cluster Incl X52046: Procollagen, type III, alpha 1/cds = (117,4511)/gb = X52046/gi = 575321/ug = Mm 4271/len = 4766/ | 5008.6 | P | 5273.9 | P | 5421.8 | P |
| | | STRA = for | | | | | | |
| 15 | 98333_at | Cluster Incl AF100956 Mus musculus major histocompatibility locus class II region, Fas-binding protein Daxx (DAXX) gene, | 3794.4 | P | 4387.4 | P | 3992.1 | P |
| | | partial cds, Bing1 (BING1), tapasin (tapasin), RalGDS-like factor (RLF), KE2 (KE2), BING4 (BING4), beta1, 3-galactosyl | | | | | | |
| | | transferase (be/STRA = for | | | | | | |
| 15 | 98342_at | Cluster Incl M93980 Mouse 24.6 kda protein mRNA, complete cds/cds = (41,685)/gb = M93980/gi = 410741/ug = Mm 43239/ | 4700.9 | P | 5201.5 | P | 5366.8 | P |
| | | len = 757/STRA = for | | | | | | |
| 15 | 98346_at | Cluster Incl AI593759 v171b05 x1 Mus musculus cDNA, 3 end/clone = IMAGE-1176561/clone_end = 3/gb = AI593759/ | 242.4 | P | 222.8 | P | 207.8 | P |
| | | gi = 4602807/ug = Mm 43514/len = 296/STRA = for | | | | | | |
| 15 | 98369_f_at | Cluster Incl AW048053: UI-M-BH1-alv-g-06-0-U1.s1 Mus musculus cDNA, 3 end/clone = UI-M-BH1-alv-g-06-0-UI/ | 3359.1 | P | 3365.1 | P | 3445.4 | P |
| | | clone_end = 3/gb = AW048053/gi = 5908582/ug = Mm 44493/len = 532/STRA = rev | | | | | | |
| 15 | 98759_f_at | Cluster Incl M28727 Tubulin alpha 2/cds = (0,1058)/gb = M28727/gi = 202224/ug = Mm 4591/len = 1198/STRA = for | 3112.3 | P | 3785 | P | 4092.8 | P |
| 15 | 98767_at | Cluster Incl AI842603: UI-M-AQ1-aed-b-11-0-UI s1 Mus musculus cDNA, 3 end/clone = UI-M-AQ1-aed-b-11-0-UI/ | 207.4 | P | 364.2 | P | 369 | P |
| | | clone_end = 3/gb = AI842603/gi = 5476818/ug = Mm 463/len = 349/STRA = rev | | | | | | |
| 15 | 98817_at | Cluster Incl Z29532 M musculus mRNA for follistatin/cds = (0,1031)/gb = Z29532/gi = 488368/ug = Mm 4913/len = 1123/ | 725.7 | P | 401 | P | 584 | P |
| | | STRA = for | | | | | | |
| 15 | 98862_at | Cluster Incl U61969 Mus musculus Wn110a mRNA, complete cds/cds = (662,1915)/gb = U61969/gi = 1546012/ug = Mm 5130/ | 195.4 | P | 284.8 | P | 353.4 | P |
| | | len = 2487/STRA = rev | | | | | | |
| 15 | 99327_at | Cluster Incl D30785 Mouse mRNA for neuropsin, complete cds/cds = (486,1268)/gb = D30785/gi = 1648847/ug = Mm 5193/ | 827.8 | P | 870.1 | P | 908 | P |
| | | len = 1322/STRA = for | | | | | | |
| 15 | 99335_at | Cluster Incl J05277: Hexokinase 1/cds = (4,2760)/gb = J05277/gi = 193841/ug = Mm 5290/len = 4198/STRA = for | 3731 | P | 3893.9 | P | 4111.3 | P |
| 15 | 99336_at | Cluster Incl U78085: Mus musculus ribosomal protein S5 mRNA, complete cds/cds = (65,679)/gb = U78085/gi = 1685070/ | 5083.6 | P | 5287.2 | P | 5474.9 | P |
| | | ug = Mm.5291/len = 763/STRA = for | | | | | | |
| 15 | 99340_at | Cluster Incl D29802 Guanine nucleotide binding protein, beta-2, related sequence 1/cds = (98,1051)/gb = D29802/ | 5024.1 | P | 5488.5 | P | 5773.7 | P |
| | | gi = 475011/ug = Mm 5305/len = 1092/STRA = for | | | | | | |
| 15 | 100297_at | Cluster Incl AA693125 vr57c08 s1 Mus musculus cDNA, 5 end/clone = IMAGE-1124750/clone_end = 5/gb = AA693125/ | 874.7 | P | 1068.5 | P | 826.9 | P |
| | | gi = 2694063/ug = Mm 86411/len = 265/STRA = for | | | | | | |
| 15 | 100307_at | Cluster Incl AA002843 mg38c08 r1 Mus musculus cDNA, 5 end/cone = IMAGE-426062/clone_end = 5/gb = AA002343/ | 252.4 | P | 216.3 | P | 206.8 | P |
| | | gi = 1446281/ug = Mm 86787/len = 235/STRA = for | | | | | | |
| 15 | 100343_f_at | Cluster Incl M28729 Tubulin alpha1/cds = (0,596)/gb = M28729/gi = 202222/ug = Mm 88110/len = 786/STRA = for | 3778.3 | P | 3595.1 | P | 3886.4 | P |
| 15 | 100379_f_at | Cluster Incl AI837905 UI-M-AL0-abq-c-01-0-UI s1 Mus musculus cDNA, 3 end/clone = UI-M-AL0-abq-c-01-0-UI/ | 4319.5 | P | 4208.4 | P | 4702 | P |
| | | clone_end = 3/gb = AI837905/gi = 5472118/ug = Mm.89082/len = 223/STRA = rev | | | | | | |
| 15 | 100380_at | Cluster Incl X91866 M musculus mRNA for H3 3A variant histone/cds = UNKNOWN/gb = X91866/gi = 1022324/ | 3948.6 | P | 4012.3 | P | 3561.7 | P |
| | | ug = Mm 89136/len = 1304/STRA = for | | | | | | |
| 15 | 100758_at | Cluster Incl AF110520 Mus musculus major histocompatibility complex region NG27, NG28, RPS28, NADH | 4155.3 | P | 4146.3 | P | 4573.8 | P |
| | | oxidoreductase, NG29, KIFC1, Fas-binding protein, BING1, tapasin, RalGDS-like, KE2, BING4, beta 1,3-galactosyl | | | | | | |
| | | transferase, and RPS18 genes, complete cds, S/STRA = for | | | | | | |
| 15 | 100780_at | Cluster Incl M73436 Ribosomal protein S4, X-linked/cds = (6,797)/gb = M73436/gi = 200863/ug = Mm.66/len = 885/STRA = for | 5853.4 | P | 6202.2 | P | 6475.9 | P |
| 15 | 101255_at | Cluster Incl X51703 Mouse mRNA for ubiquitin/cds = (104,1021)/gb = X51703/gi = 55117/ug = Mm.235/len = 1172/STRA = for | 3003 | P | 4739.1 | P | 4724.1 | P |
| 15 | 101732_at | Cluster Incl M12039 Mouse DNA homologous to the Drosophila per locus/cds = (0,2018)/gb = M12039/gi = 200288/ | 613.3 | P | 627.3 | P | 546.4 | P |
| | | ug = Mm 57054/len = 2019/STRA = for | | | | | | |
| 15 | 101781_f_at | Cluster Incl V00754 Mouse gene coding for embryonic H3 histone/cds = (0,407)/gb = V00754/gi = 51297/ug = Mm 57118/ | 1196 | P | 1269.7 | P | 1305.2 | P |
| | | len = 408/STRA = for | | | | | | |
| 15 | 101787_f_at | Cluster Incl X16672: Mouse DNA for type IIB intracistemal A-particle element encoding integrase and gag/cds = (0,323)/ | 3671.3 | P | 3761.6 | P | 3680.7 | P |
| | | gb = X16672/gi = 51499/ug = Mm 57126/len = 324/STRA = for | | | | | | |
| 15 | 102599_at | Cluster Incl X06407 Mouse mRNA for 21 kd polypeptide under translational control/cds = (100,618)/gb = X06407/gi = 52848/ | 3497.8 | P | 3607.7 | P | 4221.8 | P |
| | | ug = Mm 254/len = 816/STRA = for | | | | | | |
| 15 | 94662_at | Cluster Incl AA409766 EST01475 Mus musculus cDNA, 3 end/clone = C0010D03/clone_end = 3/gb = AA409766/ | 515.4 | P | 447.4 | P | 397.8 | P |
| | | gi = 2066390/ug = Mm 24471/len = 316/STRA = rev | | | | | | |
| 15 | 95184_f_at | Cluster Incl AA419684 vc51g02 r1 Mus musculus cDNA, 3 end/clone = IMAGE-778130/clone_end = 3/gb = AA419684/ | 353.5 | P | 477.2 | P | 482.5 | P |
| | | gi = 2080962/ug = Mm 24548/len = 243/STRA = rev | | | | | | |
| 15 | 96962_at | Cluster Incl X81987 M musculus mRNA for TAX responsive element binding protein 107/cds = (27,890)/gb = X81987/ | 4193.2 | P | 4475.4 | P | 4687.6 | P |
| | | gi = 695637/ug = Mm 588/len = 950/STRA = for | | | | | | |
| 15 | 97017_f_at | Cluster Incl AW214439 uo48c01 X1 Mus musculus cDNA, 3 end/clone = IMAGE-2645760/clone_end = 3/gb = AW214439/ | 1738.6 | P | 1866.3 | P | 2026.4 | P |
| | | gi = 6520962/ug = Mm 89021/len = 650/STRA = rev | | | | | | |
| 15 | 97077_f_at | Cluster Incl AW213991 uo46d02 x1 Mus musculus cDNA, 3 end/clone = IMAGE-2645571/clone_end = 3/gb = AW213991/ | 1745.5 | P | 1533.3 | P | 1714 | P |
| | | gi = 6520344/ug = Mm B9381/len = 468/STRA = rev | | | | | | |
| 15 | 97559_at | Cluster Inc M76131 Mouse elongation factor2 (ef-2) mRNA, 3 end/cds = (0,806)/gb = M76131/gi = 192988/ug = Mm 70787/ | 3822.5 | P | 4187 | P | 4543.3 | P |
| | | len = 1179/STRA = for | | | | | | |
| 15 | 97594_r_at | Cluster Incl C79694.C79694 Mus musculus cDNA, 3 end/clone = J0070F03/clone_end = 3/gb = C79694/gi = 2520024/ | 1292.6 | P | 1668.7 | P | 1408.5 | P |
| | | ug = Mm 89577/len = 439/STRA = rev | | | | | | |
| 15 | 97647_at | Cluster Incl M11408 Ribosomal protein S16/cds = (52,489)/gb = M11408/gi = 435544/ug = Mm 702/len = 547/STRA = for | 4801.2 | P | 5374.8 | P | 5277.6 | P |
| 15 | 98168_at | Cluster Incl M14689 Mouse surfeit locus surfeit 3 protein gene/cds = (0,800)/gb = M14689/gi = 201081/ug = Mm.725/len = 801/ | 3782 | P | 3730 | P | 3859.8 | P |
| | | STRA = for | | | | | | |
| 15 | 98169_s_at | Cluster Incl AU020229 AU020229 Mus musculus cDNA, 3 end/clone = J0529B09/clone_end = 3/gb = AU020229/ | 305.9 | P | 342.7 | P | 248.5 | P |
| | | gi = 3375813/ug = Mm 26206/len = 442/STRA = rev | | | | | | |
| 15 | 98254_f_at | Cluster Incl AW209004 un98106.x1 Mus musculus cDNA, 3 end/clone = IMAGE-2581955/clone_end = 3/gb = AW209004/ | 4380.4 | P | 4365.9 | P | 4695.9 | P |
| | | gi = 6514944/ug = Mm 91451/len = 693/STRA = rev | | | | | | |
| 15 | 100685_at | Cluster Incl M10937: Keratin complex 2, basic, gene 1/cds = (67,1953)/gb = M10937/gi = 4159805/ug = Mm.14245/len = 2341/ | 1615.6 | P | 2330.4 | P | 2555 | P |
| | | STRA = for | | | | | | |
| 15 | 100694_at | Cluster Incl U29402 Mus musculus acidic ribosomal phosphoprotein P1 mRNA, complete cds/cds = (110,454)/gb = U29402/ | 2954.4 | P | 3042.4 | P | 3211.1 | P |
| | | gi = 902557/ug = Mm 14843/len = 495/STRA = for | | | | | | |
| 15 | 100710_at | Cluster Incl Z14044 Valosin containing protein/cds = (161,2581)/gb = Z14044/gi = 55216/ug = Mm 18921/len = 3143/ | 3023.8 | P | 3046.5 | P | 3132.3 | P |
| | | STRA = for | | | | | | |
| 15 | 100711_at | Cluster Incl U12403 Mus musculus Csa-19 mRNA, complete cds/cds = (15,668)/gb = U12403/gi = 531168/ug = Mm 2424/ | 4240.5 | P | 4894.7 | P | 5294.1 | P |
| | | len = 700/STRA = for | | | | | | |
| 15 | 100718_at | Cluster Incl X56135: Mouse mRNA for prothymosin alpha/cds = (170,505)/gb = X56135/gi = 54796/ug = Mm.19187/len = 1191/ | 7380 | P | 7457.1 | P | 7042.4 | P |
| | | STRA = for | | | | | | |
| 15 | 100720_at | Cluster Incl X65553 M. musculus mRNA for poly(A) binding protein/cds = (253,2163)/gb = X65553/gi = 53753/ug = Mm 2642/ | 4344.3 | P | 4468.7 | P | 4746.5 | P |
| | | len = 2244/STRA = for | | | | | | |
| 15 | 100727_at | Cluster Incl X74856 M. musculus L28 mRNA for ribosomal protein L28/cds = (70,483)/gb = X74856/gl = 488834/ug = Mm 3111/ | 3934.7 | P | 4392.2 | P | 4483.3 | P |
| | | len = 555/STRA = for | | | | | | |
| 15 | 100729_at | Cluster Incl X80699: M. musculus L26 mRNA/cds = (47,484)/gb = X80699/gi = 565623/ug = Mm 3229/len = 545/STRA = for | 4273.1 | P | 4249.9 | P | 4607 | P |
| 15 | 100732_at | Cluster Incl X73829.M. musculus mRNA for ribosomal protein S8/cds = (6,632)/gb = X73829/gi = 313297/ug = Mm 3381/ | 5740.1 | P | 5786.3 | P | 6033.1 | P |
| | | len = 671/STRA = for | | | | | | |
| 15 | 100734_at | Cluster Incl Y00225 Murine mRNA for J1 protein, yeast ribosomal protein L3 homologue/cds = (9,1220)/gb = Y00225/ | 5371.3 | P | 5666.2 | P | 5728 | P |
| | | gi = 52740/ug = Mm.3486/len = 1276/STRA = for | | | | | | |
| 15 | 100753_at | Cluster Incl L01062 Mouse ATP synthase alpha subunit, complete cds/cds = (38,1699)/gb = L01062/gi = 192089/ | 4046 | P | 4101.1 | P | 4468 | P |
| | | ug = Mm 4069/len = 2182/STRA = for | | | | | | |
| 15 | 101128_at | Cluster Incl L06234: Mus musculus dihydropyridine sensitive skeletal muscle calcium channel mRNA, complete cds/ | 284.3 | P | 309.9 | P | 278.3 | P |
| | | cds = (116,4171)/gb = L06234/gi = 192323/ug = Mm.4418/len = 6010/STRA = for | | | | | | |
| 15 | 101129_at | Cluster Incl X83590: M. musculus mRNA for ribosomal protein L5, 3 end/cds = (0,278)/gb = X83590/gi = 619503/ug = Mm.4419/ | 2858 | P | 2905.7 | P | 2838.6 | P |
| | | len = 349/STRA = for | | | | | | |
| 15 | 101130_at | Cluster Incl X58251 Procollagen, type I, alpha 2/cds = (79,4200)/gb = X58251/gi = 50488/ug = Mm 4482/len = 4270/STRA = for | 4119.6 | P | 4042 | P | 4744 | P |
| 15 | 101137_at | Cluster Incl X76772: M musculus mRNA for ribosomal protein S3/cds = (5,736)/gb = X76772/gi = 439521/ug = Mm 4696/ | 3725.5 | P | 3616.3 | P | 4289.6 | P |
| | | len = 818/STRA = for | | | | | | |
| 15 | 101165_at | Cluster Incl W91762 MTA D08 085 A Mus musculus cDNA, 3 end/clone = MTA D08 085/clone_end = 3/gb = W91762/ | 1543.5 | P | 1453 | P | 1357 | P |
| | | gi = 1407931/ug = Mm 5000/len = 231/STRA = rev | | | | | | |
| 15 | 101179_at | Cluster Incl D50494.D-E-A-D (aspartate-glutamate-alanine-aspartate) box polypeptide 6/cds = (258,1709)/gb = D50494/ | 2942.5 | P | 3022.5 | P | 3010.6 | P |
| | | gi = 940407/ug = Mm 5082/len = 2710/STRA = for | | | | | | |
| 15 | 101207_at | Cluster Incl X52803 Mouse mRNA for cyclophilin (EC 5.2.1.8)/cds = (41,535)/gb = X52803/gi = 50620/ug = Mm 5246/len = 736/ | 6103.2 | P | 6690.7 | P | 7176.6 | P |
| | | STRA = for | | | | | | |
| 15 | 101212_at | Cluster Incl AF043285 Mus musculus ribosomal protein S7 (rpS7) gene, complete cds/cds = (79,663)/gb = AF043285/ | 4195.2 | P | 4531.3 | P | 4877.8 | P |
| | | gi = 2811283/ug = Mm 5281/len = 682/STRA = for | | | | | | |
| 15 | 101214_f_at | Cluster Incl M32599 Glyceraldehyde-3-phosphate dehydrogenase/cds = (46,1047)/gb = M32599/gi = 193423/ug = Mm 5289/ | 4157 | P | 3534.5 | P | 4263.4 | P |
| | | len = 1228/STRA = for | | | | | | |
| 15 | 101664_at | Cluster Incl Z83368 M musculus RPS3a gene/cds = (25,819)/gb = Z83368/gi = 1841931/ug = Mm 6957/len = 864/STRA = for | 6746.3 | P | 6707.6 | P | 7075 | P |
| 15 | 101680_at | Cluster Incl X05021 Munne mRNA with homology to yeast L29 ribosomal protein gene/cds = (22,468)/gb = X05021/ | 5014.1 | P | 4281.3 | P | 5194.2 | P |
| | | gi = 50320/ug = Mm 7277/len = 507/STRA = for | | | | | | |
| 15 | 102098_at | Cluster Incl AI415065 mb90h11.x1 Mus musculus cDNA, 3 end/clone = IMAGE-336741/clone_end = 3/gb = AI415065/ | 4013 | P | 4177.3 | P | 4250.8 | P |
| | | gi = 4258569/ug = Mm 42402/len = 278/STRA = rev | | | | | | |
| 15 | 102109_at | Cluster Incl U28917 Mus musculus 60S ribosomal protein (A52) mRNA, complete cds/cds = (20,661)/gb = U28917/ | 3705.6 | P | 4329.5 | P | 4192.7 | P |
| | | gi = 899444/ug = Mm 42578/len = 704/STRA = for | | | | | | |
| 15 | 102126_at | Cluster Incl X15962 Mouse mRNA for ribosomal protein S12/cds = (56,454)/gb = X15962/gi = 54005/ug = Mm 43452/len = 470/ | 3902.9 | P | 3647.3 | P | 4021.8 | P |
| | | STRA = for | | | | | | |
| 15 | 92545_f_at | Cluster Incl AB006361.Mus musculus mRNA for prostaglandin D synthetase, complete cds/cds = (0,629)/gb = AB006361/ | 369.2 | P | 474.7 | P | 285.8 | P |
| | | gi = 2317285/ug = Mm.1008/len = 789/STRA = for | | | | | | |
| 15 | 94241_at | Cluster Incl AI837229 UI-M-AK0-ade-e-06-0-UI.s1 Mus musculus cDNA, 3 end/clone = UI-M-AK0-ade-e-06-0-UI/ | 211.8 | P | 217 | P | 229 | P |
| | | clone_end = 3/gb = AI837229/gi = 5471482/ug = Mm.2235/len = 492/STRA = for | | | | | | |
| 15 | 97318_at | Cluster Incl AI846197 UI-M-AM1-agb-e-02-0-UI s1 Mus musculus cDNA, 3 end/clone = UI-M-AM1-agb-e-02-0-UI/ | 164.5 | P | 73.6 | P | 152.4 | P |
| | | clone_end = 3/gb = AI846197/gi = 5490103/ug = Mm 28109/len = 310/STRA = for | | | | | | |
| 15 | 96286_at | Cluster Incl AW060996 UI-M-BH1-amn-d-01-0-UI.s1 Mus musculus cDNA, 3 end/clone = UI-M-BH1-amn-d-01-0-UI/ | 629.7 | P | 583.4 | P | 691.9 | P |
| | | clone_end = 3/gb = AW060996/gi = 6008747/ug = Mm 29877/len = 542/STRA = for | | | | | | |
| 15 | 97483_at | Cluster Incl M62952: Mus musculus ribosomal protein L19, complete cds/cds = (0,590)/gb = M62952/gi = 198642/ | 2842.5 | P | 3681.1 | P | 3807.4 | P |
| | | ug = Mm 30806/len = 673/STRA = for | | | | | | |
| 15 | 99654_s_at | Cluster Incl AA726579 vu93a07 r1 Mus musculus cDNA, 5 end/clone = IMAGE-1209684/clone_end = 5/gb = AA726579/ | 3562.5 | P | 3580.7 | P | 3952.2 | P |
| | | gi = 2744286/ug = Mm 43751/len = 427/STRA = for | | | | | | |
| 15 | 102425_at | Cluster Incl U61362 Mus musculus groucho-related gene 1 protein (Grg1) mRNA, complete cds/cds = (54,2366)/ | 118.3 | P | 136.5 | P | 168.9 | P |
| | | gb = U61362/gi = 1420928/ug = Mm 12823/len = 2381/STRA = for | | | | | | |
| 15 | 94429_at | Cluster Incl L26479 Eukaryotic translation elongation factor 1 alpha 2/cds = (133,1524)/gb = L26479/gi = 1220409/ | 978.1 | P | 1065.9 | P | 1021.6 | P |
| | | ug = Mm 2645/len = 1782/STRA = for | | | | | | |
| 15 | 98028_at | Cluster Incl M63849 Twist gene homolog, (Drosophila)/cds = (166,786)/gb = M63649/gi = 202243/ug = Mm 3280/len = 1485/ | 319.8 | P | 29.9 | P | 351.6 | P |
| | | STRA = for | | | | | | |
| 15 | 101363_at | Cluster Incl AI644801 vb18e06 y1 Mus musculus cDNA, 5 end/clone = IMAGE-749314/clone_end = 5/gb = AI644801/ | 910 | P | 856.5 | P | 727.2 | P |
| | | gi = 4723276/ug = Mm 68974/len = 281/STRA = for | | | | | | |
| 15 | 103680_at | Cluster Incl AA683850: vr05102 r1 Mus musculus cDNA, 5 end/clone = IMAGE-1110939/clone_end = 5/gb = AA683850/ | 199 | P | 222.4 | P | 234 | P |
| | | gi = 2670436/ug = Mm 23884/len = 535/STRA = for | | | | | | |
| 15 | 103926_at | Cluster Incl AV380793: AV380793 Mus musculus cDNA, 3 end/clone = 9230102C24/clone_end = 3/gb = AV380793/ | 294.4 | P | 345.4 | P | 345 | P |
| | | gi = 6429870/ug = Mm.25082/len = 255/STRA = rev | | | | | | |
| 15 | 92388_at | Cluster Incl AW045528 UI-M-BH1-aks-c-03-0-UI s1 Mus musculus cDNA, 3 end/clone = UI-M-BH1-aks-c-03-0-UI/ | 684.7 | A | 730.1 | P | 824.3 | P |
| | | clone_end = 3/gb = AW045528/gi = 5906057/ug = Mm 4055/len = 389/STRA = for | | | | | | |
| 15 | 92527_at | Cluster Incl Z50190 Adenylate cyclase 9/cds = (42,4103)/gb = Z50190/gi = 1729772/ug = Mm 4294/len = 4457/STRA = for | 264.4 | A | 316.6 | P | 290 | P |
| 15 | 92927_at | Cluster Incl L10426 Mus musculus ets-related protein 81 (ER81) mRNA, complete cds/cds = (200,1633)/gb = L10426/ | 90.5 | P | 99.7 | P | 84.2 | P |
| | | gi = 193195/ug = Mm 4866/len = 2499/STRA = rev | | | | | | |
| 15 | 93459_s_at | Cluster Incl AW122897: UI-M-BH2.1-apa-d-11-0-UI s1 Mus musculus cDNA, 3 end/clone = UI-M-BH2 1-apa-d-11-0-UI/ | 998.6 | P | 928.4 | P | 924.1 | P |
| | | clone_end = 3/gb = AW122897/gi = 6098427/ug = Mm 68712/len = 409/STRA = for | | | | | | |
| 15 | 93964_s_at | Cluster Incl AF038995 Mus musculus putative RNA helicase RCK mRNA, partial cds/cds = (0,1359)/gb = AF038995/ | 883.6 | P | 630.8 | P | 931 | P |
| | | gi = 2745893/ug = Mm 97095/len = 1360/STRA = for | | | | | | |
| 15 | 94766_at | Cluster Incl M17878 Mouse eEF-Tu gene encoding elongation factor Tu, 5 end/cds = (80,404)/gb = M17878/gi = 192991/ | 3354.2 | P | 3466.7 | P | 3666.4 | P |
| | | ug = Mm.16317/len = 405/STRA = for | | | | | | |
| 15 | 95883_at | Cluster Incl AW048355: UI-M-BH1-akk-f-02-0-UI s1 Mus musculus cDNA, 3 end/clone = UI-M-BH1-akk-f-02-0-UI/ | 205.9 | P | 200.9 | P | 217.1 | P |
| | | clone_end = 3/gb = AW048355/gi = 5908884/ug = Mm 23966/len = 483/STRA = rev | | | | | | |
| 15 | 95964_at | Cluster Incl C77404 C77404 Mus musculus cDNA, 3 end/clone = J0030E06/clone_end = 3/gb = C77404/gi = 2517734/ | 183.4 | P | 163.7 | P | 272.6 | P |
| | | ug = Mm 24954/len = 508/STRA = rev | | | | | | |
| 15 | 96493_at | Cluster Incl AA682091 vu13g02 s1 Mus musculus cDNA, 3 end/clone = IMAGE-1180562/clone_end = 3/gb = AA682091/ | 791.9 | P | 844.7 | P | 704.8 | P |
| | | gi = 2663123/ug = Mm 25192/len = 423/STRA = rev | | | | | | |
| 15 | 99888_at | Cluster Incl U83190 Zonadhesin/cds = (0,1541)/gb = U83190/gi = 1932795/ug = Mm 7984/len = 1619/STRA = for | 479.8 | P | 437.3 | P | 461.5 | P |
| 15 | 100775_at | Cluster Incl C81070: C81070 Mus musculus cDNA, 3 end/clone = J0093A10/clone_end = 3/gb = C81070/gi = 2521400/ | 437.1 | P | 567 | P | 545 6 | P |
| | | ug = Mm.98626/len = 579/STRA = rev | | | | | | |
| 15 | 99778_at | Cluster Incl AU020239: AU020239 Mus musculus cDNA, 3 end/clone = J0529F06/clone_end = 3/gb = AU020239/ | 2094.3 | P | 2508.7 | P | 2298 | P |
| | | gi = 3375823/ug = Mm 97090/len = 434/STRA = rev | | | | | | |
| 15 | 101213_at | Cluster Incl X15267 Mouse mRNA for acidic ribosomal phosophoprotein PO/cds = (73,1026)/gb = X15267/gi = 50025/ | 2886.4 | P | 3208.6 | P | 3824.5 | P |
| | | ug = Mm 5286/len = 1094/STRA = for | | | | | | |
| 15 | AFFX-18SRNAMur/X00686_5_at | X00686 Mouse gene for 18S rRNA (_5, _M, _3 represent transcript regions 5 prime, Middle, | 574.8 | P | 697.9 | P | 492 | P |
| | | and 3 prime respectively) | | | | | | |
| 15 | 93520_at | Cluster Incl AF062655 Mus musculus plenty-of-prolines-101 mRNA, complete cds/cds = (23,2716)/gb = AF062655/ | 282.9 | P | 318.5 | M | 276.3 | P |
| | | gi = 3153820/ug = Mm 1963/len = 2957/STRA = for | | | | | | |
| 15 | 97895_f_at | Cluster Incl AW125218: UI-M-BH2 1-aps-c-06-0-UI.s1 Mus musculus cDNA, 3 end/clone = UI-M-BH2.1-aps-c-06-0-UI/ | 1329.8 | P | 1233 | A | 1418.9 | P |
| | | clone_end = 3/gb = AW125218/gi = 6100748/ug = Mm 28421/len = 319/STRA = for | | | | | | |
| 15 | 94557_at | Cluster Incl AI850893 UI-M-BH0-ajt-g-07-0-UI.s1 Mus musculus cDNA, 3 end/clone = UI-M-BH0-ajt-g-07-0-UI/clone_end = 3/ | 429.4 | P | 395.4 | A | 351.3 | P |
| | | gb = AI850893/gi = 5494799/ug = Mm 29105/len = 375/STRA = for | | | | | | |
| 15 | 96311_at | Cluster Incl M11533 Mouse myelin basic protein gene encoding 14 and 18.5 kd proteins/cds = (0,386)/gb = M11533/ | 201.4 | P | 102.3 | A | 144.9 | P |
| | | gi = 199045/ug = Mm 2992/len = 387/STRA = for | | | | | | |
| 15 | 98623_g_at | Cluster Incl X71922 Insulin-like growth factor 2/cds = (0,236)/gb = X71922/gi = 393424/ug = Mm 3862/len = 3301/STRA = for | 2133 | A | 2418.5 | P | 2544.3 | P |
| 15 | 99648_at | Cluster Incl X62772 Apolipoprotein A-II/cds = (47,355)/gb = X62772/gi = 50016/ug = Mm.43677/len = 473/STRA = for | 72.4 | P | 72.7 | P | 229.5 | P |
| 15 | 102854_s_at | Cluster Incl U03434: AT Pase, Cu++ transporting, alpha polypeptide/cds = (82,4557)/gb = U03434/gi = 451486/ug = Mm 14926/ | 127.1 | P | 167.1 | P | 222.6 | P |
| | | len = 4889/STRA = for | | | | | | |
| 15 | 103318_at | Cluster Incl M74516 GA repeat binding protein, beta 1/cds = (132,1280)/gb = M74516/gi = 193384/ug = Mm 1764/len = 2598/ | 288.5 | A | 303.9 | P | 277.7 | P |
| | | STRA = for | | | | | | |
| 15 | 103391_at | Cluster Incl AI988033: um10h12 x1 Mus musculus cDNA, 3 end/clone = IMAGE-2182727/clone_end = 3/gb = AI988033/ | 148.8 | P | 167.6 | P | 89.8 | A |
| | | gi = 5819827/ug = Mm 18654/len = 224/STRA = for | | | | | | |
| 15 | 94326_r_at | Cluster Incl AW060921 UI-M-BH1-anv-c-12-0-UI s1 Mus musculus cDNA, 3 end/clone = UI-M-BH1-anv-c-12-0-UI/ | 616.5 | P | 598.9 | A | 537.2 | P |
| | | clone_end = 3/gb = AW060921/gi = 6008672/ug = Mm 24875/len = 438/STRA = for | | | | | | |
| 15 | 97996_at | Cluster Incl U58134 Mus musculus poly(A) polymerase VI mRNA, complete cds/cds = (149,1030)/gb = U58134/gi = 1377869/ | 73.7 | P | 37.7 | M | 80.1 | P |
| | | ug = Mm 3170/len = 1046/STRA = for | | | | | | |
| 15 | 100409_at | Cluster Incl X06340.Cadherin 3/cds = (45,2513)/gb = X06340/gi = 50267/ug = Mm 4658/len = 3187/STRA = for | 438.4 | P | 593.3 | P | 336.5 | P |
| 15 | 100879_at | Cluster Incl AF093775 Mus musculus alpha-actinin 3 (Actn3) mRNA, complete cds/cds = (30,2732)/gb = AF093775/ | 533.8 | P | 596.7 | P | 736.4 | P |
| | | gi = 3694989/ug = Mm 5316/len = 2855/STRA = for | | | | | | |
| 15 | 101410_at | Cluster Incl AB000713 Mus musculus mCPE-R mRNA for CPE-receptor, complete cds/cds = (170,802)/gb = AB000713/ | 394 | P | 462.6 | A | 324.4 | P |
| | | gi = 2570126/ug = Mm 7339/len = 1783/STRA = for | | | | | | |
| 15 | 92272_at | Cluster Incl AI060709: ub41h12 r1 Mus musculus cDNA, 5 end/clone = IMAGE-1380359/clone_end = 5/gb = AI060709/ | 109.3 | P | 132.5 | P | 145.1 | P |
| | | gi = 3336132/ug = Mm 3609/len = 421/STRA = for | | | | | | |
| 15 | 92653_at | Cluster Incl AI482432.vg38e09 x1 Mus musculus cDNA, 3 end/clone = IMAGE-863656/clone_end = 3/gb = AI482432/ | 57.5 | P | 73.2 | P | 112.5 | P |
| | | gi = 4375658/ug = Mm 44063/len = 336/STRA = for | | | | | | |
| 15 | 92756_r_at | Cluster Incl X73580 M musculus mRNA for secretin/cds = (8,409)/gb = X73580/gi = 313710/ug = Mm.4723/len = 490/ | 1763.3 | P | 1651.2 | A | 1798.9 | P |
| | | STRA = for | | | | | | |
| 15 | 93413_at | Cluster Incl AF003000 Telomeric repeat binding factor 2/cds = (65,1552)/gb = AF003000/gi = 2529441/ug = Mm 6402/ | 132.2 | P | 152.4 | P | 122.6 | A |
| | | len = 2119/STRA = for | | | | | | |
| 15 | 93912_at | Cluster Incl AW047616 UI-M-BH1-akm-e-07-0-UI s1 Mus musculus cDNA, 3 end/clone = UI-M-BH1-akm-e-07-0-UI/ | 161.3 | P | 194 | P | 178.9 | P |
| | | clone_end = 3/gb = AW047616/gi = 5908145/ug = Mm.89571/len = 422/STRA = for | | | | | | |
| 15 | 94780_at | Cluster Incl AI987985 um10a01 x1 Mus musculus cDNA, 3 end/clone = IMAGE-2182632/clone_end = 3/gb = AI987985/ | 95 | A | 237.5 | P | 308.8 | P |
| | | gi = 5819779/ug = Mm 16802/len = 492/STRA = rev | | | | | | |
| 15 | 99866_at | Cluster Incl AI642417 vt02g03 x1 Mus musculus cDNA, 3 end/clone = IMAGE-1161940/clone_end = 3/gb = AI642417/ | 203.9 | P | 157.8 | P | 236.3 | P |
| | | gi = 4720892/ug = Mm.7202/len = 320/STRA = for | | | | | | |
| 15 | 101755_f_at | Cluster Incl AJ005568: Small proline-rich protein 2J/cds = (0,329)/gb = AJ005568/gi = 3093374/ug = Mm.57084/len = 330/ | 1088 | P | 1489.5 | P | 2144.6 | P |
| | | STRA = for | | | | | | |
| 15 | 101763_at | Cluster Incl AF065145 Mus musculus melatonin-related receptor (Gpr50) mRNA, complete cds/cds = (25,1800)/ | 1064.3 | P | 1031.1 | P | 1123.6 | P |
| | | gb = AF065145/gi = 3211764/ug = Mm 57095/len = 1801/STRA = for | | | | | | |
| 15 | 96416_f_at | Cluster Incl Z30939 Histone gene complex 1/cds = (0,176)/gb = Z30939/gi = 496636/ug = Mm 88398/len = 287/STRA = for | 1118.6 | P | 1117.4 | P | 1449.7 | P |
| 15 | 101631_at | Cluster Incl AF009414: Mus musculus SOX11 (Sox11) mRNA, complete cds/cds = (288,1475)/gb = AF009414/gi = 2581939/ | 368.7 | P | 230.4 | P | 305.4 | P |
| | | ug = Mm 6238/len = 2896/STRA = for | | | | | | |
| 15 | 95555_at | Cluster Incl AF030169: Mus musculus UNC-119 (Unc-119) mRNA, complete cds/cds = (69,791)/gb = AF030169/gi = 4103939/ | 204.7 | A | 124.9 | P | 157.5 | P |
| | | ug = Mm 27744/len = 1196/STRA = for | | | | | | |
| 15 | 96779_f_at | Cluster Incl AI846315 UI-M-AK1-aez-h-08-0-UI.s1 Mus musculus cDNA, 3 end/clone = UI-M-AK1-aez-h-08-0-UI/ | 212.3 | P | 231.1 | A | 241.4 | P |
| | | clone_end = 3/gb = AI846315/gi = 5490221/ug = Mm 29063/len = 345/STRA = for | | | | | | |
| 15 | 97924_at | Cluster Incl AJ132236 Mus musculus mRNA for UDP-N-acetylglucosamine 2-epimerase/N-acytmannosamine kinase/ | 285.6 | A | 414.9 | P | 462.2 | P |
| | | cds = (0,2168)/gb = AJ132236/gi = 4456672/ug = Mm 30193/len = 2169/STRA = for | | | | | | |
| 15 | 100404_at | Cluster Incl L31959: Transgene insert site 737, Insertional mutation, polycystic kidney disease/cds = (71,2545)/gb = L31959/ | 871.4 | P | 501.1 | A | 845.1 | P |
| | | gi = 499646/ug = Mm.4653/len = 3024/STRA = for | | | | | | |
| 15 | 100923_at | Cluster Incl AJ249706 Mus musculus partial mRNA for myosin X (myo 10 gene)/cds = (0,6092)/gb = AJ249706/gi = 5921504/ | 376.1 | P | 390.8 | A | 419.5 | P |
| | | ug = Mm 60590/len = 6093/STRA = for | | | | | | |
| 15 | 101855_at | Cluster Incl Y14754 Microtubule-associated protein 6/cds = (0,2720)/gb = Y14754/gi = 2769586/ug = Mm 86654/len = 2721/ | 47.9 | P | 34.9 | A | 44.3 | A |
| | | STRA = rev | | | | | | |
| 15 | 103701_at | Cluster Incl AA420331.vc51c06 r1 Mus musculus cDNA, 3 end/clone = IMAGE-778090/clone_end = 3/gb = AA420331/ | 165.8 | A | 139.2 | M | 313.7 | P |
| | | gi = 2080888/ug = Mm.24035/len 332/STRA = for | | | | | | |
| 15 | 103779_at | Cluster Incl AI852829: UI-M-BH0-ajk-b-01-0-UI s1 Mus musculus cDNA, 3 end/clone = UI-M-BH0-ajk-b-01-0-UI/ | 507.5 | P | 372.8 | P | 471.6 | A |
| | | clone_end = 3/gb = AI852829/gi = 5496735/ug = Mm 24862/len = 366/STRA = for | | | | | | |
| 15 | 104439_at | Cluster Incl AI840198: UI-M-AO0-acc-h-05-o-Ul.s1 Mus musculus cDNA, 3 end/clone = UI-M-AO0-acc-h-05-0-UI/ | 184.6 | P | 206.2 | M | 308.3 | P |
| | | clone_end = 3/gb = AI840198/gi = 5474411/ug = Mm.29271/len = 283/STRA = rev | | | | | | |
| 15 | 104442_at | Cluster Incl AI605538: vo36b03.x1 Mus musculus cDNA 3 end/clone = IMAGE-1051949/clone_end = 3/gb = AI605538/ | 262.7 | P | 312.8 | P | 246.5 | P |
| | | gi = 4614705/ug = Mm 29302/len = 286/STRA = rev | | | | | | |
| 15 | 92399_at | Cluster Incl D26532 Core binding factor alpha 2/cds = (1573,2736)/gb = D26532/gi = 493683/ug = Mm 4081/len = 4295/ | 115.3 | P | 139.1 | P | 205.3 | P |
| | | STRA = for | | | | | | |
| 15 | 92475_g_at | Cluster Incl AF083497 untitled/cds = (31,3255)/gb = AF083497/gi = 3986469/ug = Mm 42128/len = 3924/STRA = for | 51.2 | P | 63.6 | A | 114.2 | P |
| 15 | 93464_at | Cluster Incl AI581567 vw82e12.x1 Mus musculus cDNA, 3 end/clone = lMAGE-1261486/clone_end = 3/gb = AI561567/ | 23 | A | 327.3 | P | 228.1 | P |
| | | gi = 4512912/ug = Mm 6910/len = 256/STRA = for | | | | | | |
| 15 | 93466_at | Cluster Incl AF022962 Secretory protein SEC8 (S cerevisiae)/cds = (29,2944)/gb = AF022962/gi = 2547135/ug = Mm 6925/ | 130.2 | P | 127.3 | P | 139.1 | P |
| | | len = 3155/STRA = for | | | | | | |
| 15 | 95938_at | Cluster Incl AA612212 vo04a01.r1 Mus musculus cDNA, 5 end/clone = IMAGE-1040424/clone_end = 5/gb = AA612212/ | 102.6 | P | 166.9 | P | 77.9 | P |
| | | gi = 2460890/ug = Mm.24840/len = 357/STRA = for | | | | | | |
| 15 | 96513_at | Cluster Incl AA794350 vr46a11 s1 Mus musculus cDNA, 5 end/clone = IMAGE-1123676/clone_end = 5/gb = AA794350/ | 194.5 | P | 189.4 | P | 211.5 | P |
| | | gi = 2857305/ug = Mm.25583/len = 109/STRA = rev | | | | | | |
| 15 | 97744_at | Cluster Incl AI847619 UI-M-AP1-agg-d-12-0-UI s1 Mus musculus cDNA, 3 end/clone = UI-M-AP1-agg-d-12-0-UI/ | 418.3 | P | 591.2 | P | 407.6 | P |
| | | clone_end = 3/gb = AI847619/gi = 5491525/ug = Mm 40174/len = 364/STRA = rev | | | | | | |
| 15 | 102152_f_at | Cluster Incl M16724 Mouse Ig germline H-chain gene V-region (subfamily T15), exons 1 and 2/cds = (0,187)/gb = M16724/ | 435.1 | P | 441 | P | 389.9 | A |
| | | gi = 196262/ug = Mm 46800/len = 188/STRA = for | | | | | | |
| 15 | AFFX-18SRNAMur/X00686_3_at | X00686 Mouse gene for 18S rRNA (_5, _M, _3 represent transcript regions 5 prime, Middle, | 330.9 | P | 594 | P | 312.4 | P |
| | | and 3 prime respectively) | | | | | | |
| 15 | 93087_r_at | Cluster lncl AA267185 mz92g02 r1 Mus musculus cDNA, 5 end/clone = IMAGE-720914/clone_end = 5/gb = AA267185/ | 345.9 | A | 488.4 | P | 378.2 | M |
| | | gi = 1903945/ug = Mm 16253/len = 229/STRA = for | | | | | | |
| 15 | 95138_at | Cluster Incl AI836568 UI-M-AP0-abf-a-06-0-UI s1 Mus musculus cDNA, 3 end/clone = UI-M-AP0-abf-a-06-0-UI/ | 124.6 | A | 196.6 | P | 238.4 | P |
| | | clone_end = 3/gb = AI836568/gi = 5470781/ug = Mm 29432/len = 285/STRA = for | | | | | | |
| 15 | 104109_at | Cluster Incl AI853773 UI-M-BH0-ajg-e-03-0-UI s1 Mus musculus cDNA, 3 end/clone = UI-M-BH0-ajg-e-03-0-UI/ | 141.7 | P | 136.4 | A | 131.8 | P |
| | | clone_end = 3/gb = AI853773/gi = 5497679/ug = Mm 21912/len = 453/STRA = for | | | | | | |
| 15 | 104590_at | Cluster Incl L13171 Myocyte enhancer factor 2C/cds = UNKNOWN/gb = L13171/gi = 293728/ug = Mm 24001/len = 1656/ | 336.5 | P | 381.9 | P | 468.1 | P |
| | | STRA = for | | | | | | |
| 15 | 97355_at | Cluster Incl AW050020 UI-M-BH1-amm-a-08-0-UI s1 Mus musculus cDNA, 3 end/clone = UI-M-BH1-amm-a-08-0-UI/ | 97.3 | P | 63.1 | P | 130.6 | A |
| | | clone_end = 3/gb = AW050020/gi = 5910549/ug = Mm 29632/len = 458/STRA = for | | | | | | |
| 15 | 97975_at | Cluster Incl D85612 Nuclear factor of activated T-cells, cytoplasmic 3/cds = (7,3234)/gb = D85612/gi = 1906311/ | 431.8 | P | 373.2 | P | 431.2 | P |
| | | ug = Mm 3107/len = 3638/STRA = for | | | | | | |
| 15 | 103460_at | Cluster Incl AI849939: UI-M-BG0-ahz-g-03-0-UI s1 Mus musculus cDNA, 3 end/clone = UI-M-BG0-ahz-g-03-0-UI/ | 69.1 | P | 348.8 | A | 474.4 | P |
| | | clone_end = 3/gb = AI849939/gi = 5493845/ug = Mm 21697/len = 440/STRA = for | | | | | | |
| 15 | 103925_at | Cluster Incl AW120605 UI-M-BH2.3-anx-f-01-0-UI.s1 Mus musculus cDNA, 3 end/clone = UI-M-BH2 3-anx-f-01-0-UI/ | 290.1 | A | 371.1 | P | 370.3 | P |
| | | clone_end = 3/gb = AW120605/gi = 6095938/ug = Mm 25080/len = 151/STRA = for | | | | | | |
| 15 | 94104_at | Cluster Incl AF051911 Telomerase reverse transcriptase/cds = (29,3397)/gb = AF051911/gi = 3005591/ug= Mm 10109/ | 247.2 | P | 233 | M | 250.1 | P |
| | | len = 3426/STRA = for | | | | | | |
| 15 | 98303_at | Cluster Incl X79131: M. musculus mRNA for IB3/5-polypeptide/cds = (833,2869)/gb = X79131/gi = 485857/ug = Mm 42143/ | 364.4 | P | 359.8 | P | 379.5 | P |
| | | len = 3008/STRA = for | | | | | | |
| 15 | 99439_at | Cluster Incl X67735.MAS1 oncogene/cds = (0,974)/gb = X67735/gi = 53011/ug = Mm 57182/len = 975/STRA = for | 355.6 | M | 347.4 | M | 365.8 | P |
| 15 | 95869_at | Cluster Incl C78041 C78041 Mus musculus cDNA, 3 end/clone = J0041H05/clone_end = 3/gb = C78041/gi = 2518371/ | 66.1 | P | 65.9 | P | 67.5 | P |
| | | ug = Mm.24980/len = 538/STRA = rev | | | | | | |
| 15 | 95879_at | Cluster Incl C79052 C79052 Mus musculus cDNA, 3 end/clone = J0059F07/clone_end = 3/gb = C79052/gi = 2519382/ | 44.2 | P | 29.2 | P | 60.3 | P |
| | | ug = Mm 25008/len = 608/STRA = rev | | | | | | |
| 15 | 100686_at | Cluster Incl M20632 Mouse LLRep3 protein mRNA from a repetitive element, complete cds/cds = (1022,1687)/gb = M20632/ | 2330.3 | A | 2978.1 | P | 3084.3 | P |
| | | gi = 200811/ug = Mm 1129/len = 1728/STRA = for | | | | | | |
| 15 | AFFX-18SRNAMur/X00686_M_at | X00686 Mouse gene for 18S rRNA (_5, _M, _3 represent transcript regions 5 prime, Middle, | 408.5 | P | 398.9 | P | 413.5 | P |
| | | and 3 prime respectively) | | | | | | |
| 15 | 99980_at | Cluster Incl M35986 Mouse homeobox-6 1 protein (Hox-6.1) mRNA, 3 end/cds = (0,317)/gb = M35986/gi = 193981/ | 202.3 | A | 199.9 | P | 300.8 | A |
| | | ug = Mm 4444/len = 318/STRA = for | | | | | | |
| 15 | 102695_at | Cluster Incl AF021335 Mus musculus T cell receptor gamma locus, TCR gamma 2 and gamma 4 gene clusters/ | 366.7 | A | 318.7 | M | 311.2 | P |
| | | cds = (0,571)/gb = AF021335/gi = 2444469/ug = Mm 14145/len = 572/STRA = for | | | | | | |
| 15 | 102865_at | Cluster Incl U58993 MAD homolog 5 (Drosophila)/cds = (266,1663)/gb = U58993/gi = 1518646/ug = Mm.1470/len = 2185/ | 135.3 | M | 166 | P | 163.5 | P |
| | | STRA= for | | | | | | |
| 15 | 102871_at | Cluster Incl L77867 Mus musculus MEP mRNA, complete cds/cds = (202,3246)/gb = L77867/gi = 1911182/ug = Mm.1480/ | 95.2 | A | 38.9 | P | 182.5 | A |
| | | len = 3464/STRA = for | | | | | | |
| 15 | 103229_at | Cluster Incl U64828 Mus musculus steroid receptor coactivator-1 (mSRC-1) mRNA, complete cds/cds = (625,4842)/ | 146.2 | A | 189.6 | A | 133.1 | P |
| | | gb = U64828/gi = 1490875/ug = Mm 2028/1en = 5169/STRA = for | | | | | | |
| 15 | 104010_at | Cluster Incl U62906: Mus musculus zinc finger protein 94 (Zfp94) mRNA, complete cds/cds = (180,1742)/gb = U62906/ | 122.6 | A | 155.9 | P | 195.2 | P |
| | | gi = 4097496/ug = Mm.26782/len = 1997/STRA = for | | | | | | |
| 15 | 92364_at | Cluster Incl AB028499 Mus musculus mRNA for Flamingo 1, complete cds/cds = (0,8762)/gb = AB028499/gi = 5832710/ | 518.7 | A | 733.6 | P | 931.7 | P |
| | | ug = Mm 39728/len = 8950/STRA = for | | | | | | |
| 15 | 93900_at | Cluster Incl AW050268 UI-M-BH1-ans-d-03-0-UI.s1 Mus musculus cDNA, 3 end/clone = UI-M-BH1-ans-d-03-0-UI/ | 541.5 | A | 642.5 | P | 835.4 | P |
| | | clone_end = 3/gb = AW050268/gi = 5910797/ug = Mm 89316/len = 221/STRA = for | | | | | | |
| 15 | 95284_at | Cluster Incl AI662509 mt33c08 x1 Mus musculus cDNA, 3 end/clone = IMAGE-822862/clone_end = 3/gb = AI662509/ | 66.7 | A | 100.5 | A | 120.4 | P |
| | | gi = 4766092/ug = Mm.1850/len = 231/STRA = for | | | | | | |
| 15 | 95995_at | Cluster Incl C80108 C80108 Mus musculus cDNA, 3 end/clone = J0076F01/clone_end = 3/gb = C80108/gi = 2520438/ | 190.8 | P | 194.6 | A | 214.5 | P |
| | | ug = Mm 25061/1en = 349/STRA = rev | | | | | | |
| 15 | 100282_at | Cluster Incl Z23107 5-hydroxytryptamine (serotonin) receptor 7/cds = (108,1454)/gb = Z23107/gi = 396586/ug = Mm 8053/ | 225.9 | P | 506.5 | P | 434.4 | A |
| | | len = 1540/STRA = for | | | | | | |
| 15 | 95872_at | Cluster Incl C78535: C78535 Mus musculus cDNA, 3 end/clone = J0050H08/clone_end = 3/gb = C78535/gi = 2518865/ | 48 | M | 86.4 | A | 112.3 | P |
| | | ug= Mm.24993/len = 576/STRA = rev | | | | | | |
| 15 | 101159_at | Cluster Incl Z23066: Microphthalmia-associated transcription factor/cds = (129,1388)/gb = Z23066/gi = 396739/ug = Mm 4978/ | 118 | P | 224.6 | P | 178.6 | P |
| | | len = 1922/STRA = for | | | | | | |
| 15 | 101618_r_at | Cluster Incl U59758 Mus musculus p53-variant (p53) mRNA, partial cds/cds = (0,161)/gb= U59758/gi = 1399879/ | 528.1 | P | 437.3 | P | 548.4 | P |
| | | ug = Mm.6186/len = 846/STRA = for | | | | | | |
| 15 | 103348_at | Cluster Incl AI837688 UI-M-AK0-adj-c-07-0-UI.s1 Mus musculus cDNA, 3 end/clone = UI-M-AK0-adj-c-07-0-UI/ | 158.9 | A | 431.4 | P | 307.8 | A |
| | | clone_end = 3/gb = AI837688/gi = 5471901/ug = Mm 18311/len = 392/STRA = for | | | | | | |
| 15 | 98507_at | Cluster Incl AI834950 UI-M-AM1-afw-b-05-0-UI.s1 Mus musculus cDNA, 3 end/clone = UI-M-AM1-afw-b-05-0-UI/ | 179.8 | P | 204.7 | P | 203.8 | A |
| | | clone_end = 3/gb = AI834950/gi = 5469163/ug = Mm 35822/len = 315/STRA = rev | | | | | | |
| 15 | 100021_at | Cluster Incl M17640 Mus musculus acetylcholine receptor alpha-subunit mRNA, complete cds/cds = (104,1477)/ | 81.6 | P | 88.5 | A | 130.5 | P |
| | | gb = M17640/gi = 2073542/ug = Mm 4583/len = 1860/STRA = for | | | | | | |
| 15 | 101381_at | Cluster Incl D86066 Mus musculus mRNA for mouse rabaptin-5, complete cds/cds = (50,2638)/gb = D86066/gi = 2329848/ | 53.6 | P | 45 | M | 76.1 | A |
| | | ug = Mm 7087/len = 3095/STRA = for | | | | | | |
| 15 | 104195_at | Cluster Incl AA939440: vz49d01.r1 Mus musculus cDNA, 5 end/clone = IMAGE-1329793/clone_end = 5/gb = AA939440/ | 290.3 | P | 218.2 | A | 308 | P |
| | | gi = 3100217/ug = Mm 27503/len = 379/STRA = for | | | | | | |
| 15 | 104415_at | Cluster Incl AA833293 ud05d05 r1 Mus musculus cDNA, 5 end/clone = IMAGE-1434249/clone_end = 5/gb= AA833293/ | 151.8 | P | 148 | P | 121.3 | A |
| | | gi = 2907021/ug = Mm 28907/len = 548/STRA = for | | | | | | |
| 15 | 93155_at | Cluster Incl AI847069 UI-M-AP1-agn-c-04-0-UI s1 Mus musculus cDNA, 3 end/clone = UI-M-AP1-agn-c-04-0-UI/ | 39.2 | P | 41.8 | M | 51.9 | M |
| | | clone_end = 3/gb = AI847069/gi = 5490975/ug = Mm.5263/len = 263/STRA = for | | | | | | |
| 15 | 97711_at | Cluster Incl AI606967 vw37a01 x1 Mus musculus cDNA, 3 end/clone = IMAGE-1245960/clone_end = 3/gb = AI606967/ | 116.1 | P | 92.2 | A | 86 | P |
| | | gi = 4616134/ug = Mm 38364/len = 387/STRA = rev | | | | | | |
| 15 | 99908_at | Cluster Incl AB010557 Mus musculus mRNA for Pax4, complete cds/cds = (206,1255)/gb = AB010557/gi = 2809092/ | 431.8 | A | 470.1 | A | 447.1 | P |
| | | ug = Mm 8026/len = 1295/STRA = for | | | | | | |
| 15 | 100749_at | Cluster Incl AA414976: vc49e02 r1 Mus musculus cDNA, 3 end/clone = IMAGE-777914/clone_end = 3/gb = AA414976/ | 184.9 | A | 240.3 | P | 157.6 | A |
| | | gi = 2075103/ug = Mm 3802/len = 332/STRA = rev | | | | | | |
| 16 | 92567_at | Cluster Incl L02918 Mouse procollagen type V alpha 2 (Col5a-2) mRNA, complete cds/cds = (0,4493)/gb = L02918/ | 2726.4 | P | 1413.7 | P | 1650.3 | P |
| | | gi = 309180/ug = Mm 10299/len = 4494/STRA = for | | | | | | |
| 16 | 92587_at | Cluster Incl L29123 Mus musculus adrenodoxin mRNA, complete cds/cds = (88,654)/gb = L29123/gi = 457297/ug = Mm 1061/ | 663 | P | 571.2 | P | 558.6 | P |
| | | len = 883/STRA = for | | | | | | |
| 16 | 92795_at | Cluster Incl M72414 Microtubule-associated protein 4/cds = (490,3867)/gb = M72414/gi = 456554/ug = Mm 12625/len = 6082/ | 496.5 | P | 351.9 | P | 461 | P |
| | | STRA = for | | | | | | |
| 16 | 92816_r_at | Cluster Incl X03039.Eukaryotic translation Initiation factor 4A1/cds = UNKNOWN/gb = X03039/gi = 50812/ug = Mm 12858/ | 2055.3 | P | 1797.1 | P | 1692 | P |
| | | len = 1407/STRA = for | | | | | | |
| 16 | 92833_at | Cluster Incl L07645 Histidine ammonia tyase/cds = UNKNOWN/gb= L07645/gi = 193751/ug = Mm 13000/len = 2281/ | 2213.8 | P | 1565.6 | P | 1724.3 | P |
| | | STRA = for | | | | | | |
| 16 | 93029_at | Cluster Incl U68564 Isocitrale dehydrogenase 3 (NAD+), gamma/cds = (42,1223)/gb = U68564/gi = 1546828/ug = Mm 14825/ | 834.8 | P | 715.8 | P | 712.5 | P |
| | | len = 1335/STRA = for | | | | | | |
| 16 | 93316_at | Cluster Incl AB017026 Mus musculus mRNA for oxysterol-binding protein, complete cds/cds = (827,2059)/gb = AB017026/ | 535 | P | 406.3 | P | 409.9 | P |
| | | gi = 3551522/ug = Mm 18502/len = 2931/STRA = for | | | | | | |
| 16 | 93333_at | Cluster Incl U05333 Mus musculus co-chaperonin cofactor A mRNA, complete cds/cds = (7,333)/gb = U05333/gi = 1117784/ | 496 | P | 392.7 | P | 507.6 | P |
| | | ug = Mm 1863/len = 508/STRA = for | | | | | | |
| 16 | 93571_at | Cluster Incl M74773 Beta-spectrin 2, non-erythrocytic/cds = (366,7457)/gb = M74773/gi = 598330/ug = Mm 20405/len = 7524/ | 1607.7 | P | 1731.5 | P | 1501 | P |
| | | STRA = for | | | | | | |
| 16 | 93574_at | Cluster Incl AF036164 Mus musculus pigment epithelium-derived factor (PEDF) mRNA, complete cds/cds = (33,1286)/ | 397.5 | P | 244.8 | P | 464.3 | P |
| | | gb = AF036164/gi = 3808220/ug = Mm 2044/len = 1648/STRA = for | | | | | | |
| 16 | 93731_at | Cluster Incl AF090334 Mus musculus FK506 binding protein (Fkbp63) mRNA, complete cds/cds = (74,1786)/gb = AF090334/ | 1470.7 | P | 975.1 | P | 1004.5 | P |
| | | gi = 3860028/ug = Mm 20943/len = 2945/STRA = for | | | | | | |
| 16 | 93750_at | Cluster Incl J04953 Gelsolin/cds = (18,2213)/gb = J04953/gi = 193463/ug = Mm 21109/len = 2438/STRA = for | 2505.6 | P | 1839.6 | P | 2086.2 | P |
| 16 | 93754_at | Cluster Incl AF030343 Mus musculus peroxisomal/mitochondrial dienoyl-CoA isomerase ECH1p (Ech1) mRNA, complete | 747.8 | P | 486.5 | P | 675.5 | P |
| | | cds/cds = (23,1006)/gb = AF030343/gi = 2606085/ug = Mm 2112/len = 1138/STRA = for | | | | | | |
| 16 | 94031_at | Cluster Incl X95403 M musculus mRNA for GTP-binding protein/cds = (207,845)/gb = X95403/gi = 1177546/ug = Mm.21905/ | 1094.8 | P | 1246.2 | P | 969.2 | P |
| | | len = 1008/STRA = for | | | | | | |
| 16 | 94059_at | Cluster Incl AI852524 UI-M-BH0-aiw-d-04-0-UI.s1 Mus musculus cDNA, 3 end/clone = UI-M-BH0-aiw-d-04-0-UI/ | 996.3 | P | 401.5 | P | 473.3 | P |
| | | clone_end = 3/gb = AI852524/gi = 5496430/ug = Mm 22029/len = 382/STRA = for | | | | | | |
| 16 | 94233_at | Cluster Incl AW048642 UI-M-BH1-amd-d-06-0-UI s1 Mus musculus cDNA, 3 end/clone = UI-M-BH1-amd-d-06-0-UI/ | 241.1 | P | 308.4 | P | 199 | P |
| | | clone_end = 3/gb = AW048642/gi = 5909171/ug = Mm.22294/len = 480/STRA = for | | | | | | |
| 16 | 94269_at | Cluster Incl L40934 Mus musculus rab6/rab5-associated protein (rab6) mRNA, partial cds/cds = (0,307)/gb = L40934/ | 2127.6 | P | 1741.5 | P | 2515.4 | P |
| | | gi = 722666/ug = Mm 22473/len = 308/STRA = for | | | | | | |
| 16 | 94284_at | Cluster Incl AW122731 UI-M-BH2 2-aot-e-08-0-UI s1 Mus musculus cDNA, 3 end/clone = UI-M-BH2 2-aot-e-08-0-UI/ | 769.7 | P | 809.6 | P | 839.6 | P |
| | | clone_end = 3/gb = AW122731/gi = 6098261/ug = Mm 22560/len = 204/STRA = for | | | | | | |
| 16 | 94289_r_at | Cluster Incl AI851574 UI-M-BH0-aki-f-10-0-UI s1 Mus musculus cDNA, 3 end/clone = UI-M-BH0-aki-f-10-0-UI/clone_end = 3/ | 912.2 | P | 436 | P | 771.5 | P |
| | | gb = AI851574/gi = 5495480/ug = Mm 22575/len = 459/STRA = for | | | | | | |
| 16 | 94870_f_at | Cluster Incl AW124226 UI-M-BH2 1-aph-d-10-0-UI.s1 Mus musculus cDNA, 3 end/clone = UI-M-BH2.1-aph-d-10-0-UI/ | 269.7 | P | 124.3 | P | 161.9 | P |
| | | clone_end = 3/gb = AW124226/gi = 6099756/ug = Mm 23755/len = 382/STRA = for | | | | | | |
| 16 | 94872_at | Cluster Incl Y08135 M musculus mRNA for ASM-like phosphodiesterase 3a/cds = (92,1429)/gb = Y08135/gi = 1552349/ | 317.2 | P | 261.6 | P | 281.8 | P |
| | | ug = Mm 2379/len = 1758/STRA = for | | | | | | |
| 16 | 94878_at | Cluster Incl AW125224 UI-M-BH2 1-aps-d-02-0-UI s1 Mus musculus cDNA, 3 end/clone = UI-M-BH2 1-aps-d-02-0-UI/ | 550.1 | P | 620.9 | P | 600 | P |
| | | clone_end = 3/gb = AW125224/gi = 6100754/ug = Mm 23839/len = 356/STRA = for | | | | | | |
| 16 | 94889_at | Cluster Incl AW121235 UI-M-BH2 3-aok-f-05-0-UI s1 Mus musculus cDNA, 3 end/clone = UI-M-BH2 3-aok-f-05-0-UI/ | 1850 | P | 1715.8 | P | 1993 | P |
| | | clone_end = 3/gb = AW121235/gi = 6096568/ug = Mm 23943/len = 293/STRA = for | | | | | | |
| 16 | 94892_r_at | Cluster Incl M27938: Male enhanced antigen 1/cds = (80,604)/gb = M27938/gi = 199115/ug = Mm.2395/len = 841/STRA = for | 1093.9 | P | 1005.1 | P | 1004.1 | P |
| 16 | 94899_at | Cluster Incl U73200. Mus musculus p116Rip mRNA, complete cds/cds = (191,3265)/gb = U73200/gi = 1657836/ug = Mm 2402/ | 1832 | P | 1898.9 | P | 1903.1 | P |
| | | len = 4010/STRA = for | | | | | | |
| 16 | 95411_at | Cluster Incl AB025407 Mus musculus mRNA for sid2057p, complete cds/cds = (83,628)/gb = AB025407/gi = 5931562/ | 1375.2 | P | 1219.8 | P | 1114.2 | P |
| | | ug = Mm 24231/len = 1400/STRA = for | | | | | | |
| 16 | 96049_at | Cluster Incl X53928: M musculus mRNA for PGI (biglycan)/cds = (234,1343)/gb = X53928/gi = 53666/ug = Mm 2608/ | 3229.2 | P | 2622.4 | P | 2744.3 | P |
| | | len = 1964/STRA = for | | | | | | |
| 16 | 96629_at | Cluster Incl X04097: Mouse kidney lestosterone-regulated RP2 mRNA/cds = (41,1114)/gb = X04097/gi = 54003/ | 398.5 | P | 289.7 | P | 370.4 | P |
| | | ug = Mm.27194/len = 2005/STRA = for | | | | | | |
| 16 | 96634_at | Cluster Incl AI850090: UI-M-BG0-aia-g-01-0-UI.s1 Mus musculus cDNA, 3 end/clone = UI-M-BG0-aia-g-01-0-UI/ | 343 | P | 203.1 | P | 247.6 | P |
| | | clone_end = 3/gb = AI850090/gi = 5493996/ug = Mm 27227/len = 481/STRA = for | | | | | | |
| 16 | 96641_at | Cluster Incl AW060674 UI-M-BH1-anl-d-04-0-UI s1 Mus musculus cDNA, 3 end/clone = UI-M-BH1-anl-d-04-0-UI/ | 530.2 | P | 398.1 | P | 407.3 | P |
| | | clone_end = 3/gb = AW060674/gi = 6008425/ug = Mm 27285/len = 486/STRA = for | | | | | | |
| 16 | 96708_at | Cluster Incl AW120643 UI-M-BH2 3-anz-f-05-0-UI.s1 Mus musculus cDNA, 3 end/clone = UI-M-BH2 3-anz-f-05-0-UI/ | 1316.4 | P | 677.6 | P | 1152.5 | P |
| | | clone_end = 3/gb = AW120643/gi = 6096016/ug = Mm 27606/len = 505/STRA = for | | | | | | |
| 16 | 97277_at | Cluster Incl AI844179 UI-M-AL1-ahk-a-04-0-UI s1 Mus musculus cDNA, 3 end/clone = UI-M-AL1-ahk-a-04-0-UI/ | 341.4 | P | 160.7 | P | 147 | P |
| | | clone_end = 3/gb = AI844179/gi = 5488085/ug = Mm 27951/len = 281/STRA = for | | | | | | |
| 16 | 97841_at | Cluster Incl AI838663 UI-M-AO0-aca-d-07-0-UI.s1 Mus musculus cDNA, 3 end/clone = UI-M-AO0-aca-d-07-0-UI/ | 1049.4 | P | 955.1 | P | 1137.9 | P |
| | | clone_end = 3/gb = AI838663/gi = 5472876/ug = Mm 28258/len = 368/STRA = for | | | | | | |
| 16 | 97901_at | Cluster Incl X60831 Mouse ubf gene for transcription factor UBF/cds = UNKNOWN/gb = X60831/gi = 55115/ug = Mm 2845/ | 486.5 | P | 502.3 | P | 446.7 | P |
| | | len = 3031/STRA = for | | | | | | |
| 16 | 94454_at | Cluster Incl AF085348 Mus musculus proline-nch protein (Bprp) mRNA, complete cds/cds = (15,521)/gb = AF085348/ | 931.6 | P | 1066.9 | P | 1008 | P |
| | | gi = 3549610/ug = Mm 28797/len = 1779/STRA = for | | | | | | |
| 16 | 94531_at | Cluster Incl AW124582: UI-M-BH2 1-apt-e-11-0-UI s1 Mus musculus cDNA, 3 end/clone = UI-M-BH2 1-apt-e-11-0-UI/ | 460.3 | P | 300.9 | P | 430 | P |
| | | clone_end = 3/gb = AW124582/gi = 6100112/ug = Mm 29042/len = 360/STRA = for | | | | | | |
| 16 | 94549_at | Cluster Incl AI315650: uj31d03 x1 Mus musculus cDNA, 3 end/clone = IMAGE-1921541/clone_end = 3/gb = AI315650/ | 63.2 | P | 51.7 | P | 65.1 | P |
| | | gi = 4030917/ug = Mm 29078/len = 514/STRA = for | | | | | | |
| 16 | 95054_at | Cluster Incl AI849620: UI-M-AH1-agr-h-02-0-UI s1 Mus musculus cDNA, 3 end/clone = UI-M-AH1-agr-h-02-0-UI/ | 409.6 | P | 401.4 | P | 423.8 | P |
| | | clone_end = 3/gb = AI849620/gi = 5493526/ug = Mm 29144/len = 340/STRA = for | | | | | | |
| 16 | 95074_at | Cluster Incl AW125309: UI-M-BH2.1-apy-d-05-0-UI s1 Mus musculus cDNA, 3 end/clone = UI-M-BH2 1-apy-d-05-0-UI/ | 308.8 | P | 304.8 | P | 268.8 | P |
| | | clone_end = 3/gb = AW125309/gi = 6100839/ug = Mm 29198/len = 276/STRA = for | | | | | | |
| 16 | 95136_at | Cluster Incl AW049185: UI-M-BH1-amq-g-01-0-UI.s1 Mus musculus cDNA, 3 end/clone = UI-M-BH1-amq-g-01-0-UI/ | 488.5 | P | 336.3 | P | 472.5 | P |
| | | clone_end = 3/gb = AW049185/gi = 5909714/ug = Mm 29430/len = 483/STRA = for | | | | | | |
| 16 | 95701_at | Cluster Incl AW124069: UI-M-BH2.1-apn-f-11-0-UI.s1 Mus musculus cDNA, 3 end/clone = UI-M-BH2 1-apn-f-11-0-UI/ | 199.3 | P | 214.8 | P | 185.5 | P |
| | | clone_end = 3/gb = AW124069/gi = 6099599/ug = Mm 29690/len = 390/STRA = for | | | | | | |
| 16 | 95732_at | Cluster Incl AW047746: UI-M-BH1-alk-b-11-0-UI s1 Mus musculus cDNA, 3 end/clone = UI-M-BH1-alk-b-11-0-UI/ | 671.6 | P | 498.9 | P | 577.9 | P |
| | | clone_end = 3/gb = AW047746/gi = 5908275/ug = Mm 29743/len = 406/STRA = for | | | | | | |
| 16 | 95737_at | Cluster Incl AW120814: UI-M-BH2.3-aoa-b-07-0-UI s1 Mus musculus cDNA, 3 end/clone = UI-M-BH2 3-aoa-b-07-0-UI/ | 827.9 | P | 755.1 | P | 706.1 | P |
| | | clone_end = 3/gb = AW120814/gi = 6096147/ug = Mm 29750/len = 314/STRA = for | | | | | | |
| 16 | 96338_at | Cluster Incl AW125059: UI-M-BH2.1-apx-c-07-0-UI s1 Mus musculus cDNA, 3 end/clone = UI-M-BH2 1-apx-c-07-0-UI/ | 1065.1 | P | 883.7 | P | 1169.3 | P |
| | | clone_end = 3/gb = AW125059/gi = 6100589/ug = Mm 29978/len = 448/STRA = for | | | | | | |
| 16 | 96845_at | Cluster Incl AI849577 UI-M-AH1-agr-d-03-0-UI.s1 Mus musculus cDNA, 3 end/clone = UI-M-AH1-agr-d-03-0-UI/ | 243.5 | P | 145.5 | P | 197.7 | P |
| | | clone_end = 3/gb = AI849577/gi = 5493483/ug = Mm 30023/len = 242/STRA = for | | | | | | |
| 16 | 96894_at | Cluster Incl AW122685 UI-M-BH2.2-aot-a-06-0-UI s1 Mus musculus cDNA, 3 end/clone = UI-M-BH2 2-aot-a-06-0-UI/ | 421.9 | P | 386.3 | P | 467 | P |
| | | clone_end = 3/gb = AW122685/gi = 6098215/ug = Mm 30103/len = 445/STRA = for | | | | | | |
| 16 | 96934_at | Cluster Incl AW049074 UI-M-BH1-amt-d-01-0-UI s1 Mus musculus cDNA, 3 end/clone = UI-M-BH1-aml-d-01-0-UI/ | 1037.5 | P | 849.7 | P | 826.6 | P |
| | | clone_end = 3/gb = AW049074/gi = 5909603/ug = Mm 30179/len = 285/STRA = for | | | | | | |
| 16 | 97463_g_at | Cluster Incl AI847584 UI-M-AP1-agg-a-06-0-UI s1 Mus musculus cDNA, 3 end/clone = UI-M-AP1-agg-a-06-0-UI/ | 2659 | P | 2426.9 | P | 2238.6 | P |
| | | clone_end = 3/gb = AI847584/gi = 5491490/ug = Mm 3035/len = 340/STRA = for | | | | | | |
| 16 | 98047_at | Cluster Incl AI851583 UI-M-BH0-aki-g-07-0-UI s1 Mus musculus cDNA, 3 end/clone = UI-M-BH0-aki-g-07-0-UI/ | 388.7 | P | 273.7 | P | 373.2 | P |
| | | clone_end = 3/gb = AI851583/gi = 5495489/ug = Mm.34261/len = 460/STRA = for | | | | | | |
| 16 | 98070_at | Cluster Incl AI844794 UI-M-AH1-agx-a-02-0-UI s1 Mus musculus cDNA, 3 end/clone = UI-M-AH1-agx-a-02-0-UI/ | 731.8 | P | 885.5 | P | 848.5 | P |
| | | clone_end = 3/gb = AI844794/gi = 5488700/ug = Mm 34456/len = 394/STRA = for | | | | | | |
| 16 | 98522_at | Cluster Incl AI839158 UI-M-AO0-ach-f-03-0-UI s1 Mus musculus cDNA, 3 end/clone = UI-M-AO0-ach-f-03-0-UI/ | 1461.9 | P | 1092 | P | 1293.5 | P |
| | | clone_end = 3/gb = AI839158/gi = 5473371/ug = Mm 35812/len = 419/STRA = for | | | | | | |
| 16 | 98528_at | Cluster Incl AI854901 UI-M-BH0-akb-h-06-0-UI s1 Mus musculus cDNA, 3 end/clone = UI-M-BH0-akb-h-06-0-UI/ | 1543.3 | P | 1050.3 | P | 1283.9 | P |
| | | clone_end = 3/gb = AI854901/gi = 5498807/ug = Mm 35837/len = 398/STRA = for | | | | | | |
| 16 | 98625_s_at | Cluster Incl M84147 Mouse alcohol dehydrogenase-B2 (Adh-2) mRNA, putative cds/cds = (39,1163)/gb = M84147/ | 692.7 | P | 638.2 | P | 655.4 | P |
| | | gi = 191721/ug = Mm.3874/len = 1360/STRA = for | | | | | | |
| 16 | 98633_at | Cluster Incl AI854863 UI-M-BH0-akb-d-12-0-UI s1 Mus musculus cDNA, 3 end/clone = UI-M-BH0-akb-d-12-0-UI/ | 787.5 | P | 629.5 | P | 621 | P |
| | | clone_end = 3/gb = AI854863/gi = 5498769/ug = Mm 38801/len = 419/STRA = for | | | | | | |
| 16 | 98635_at | Cluster Incl AI854629 UI-M-BH0-ake-e-11-0-UI s1 Mus musculus cDNA, 3 end/clone = UI-M-BH0-ake-e-11-0-UI/ | 584.6 | P | 525.4 | P | 481.1 | P |
| | | clone_end = 3/gb = AI854629/gi = 5498535/ug = Mm 38802/len = 492/STRA = for | | | | | | |
| 16 | 99176_at | Cluster Incl AI843393: UI-M-AQ1-adz-g-04-0-UI s1 Mus musculus cDNA, 3 end/clone = UI-M-AQ1-adz-g-04-0-UI/ | 341.4 | P | 342.5 | P | 336.7 | P |
| | | clone_end = 3/gb = AI843393/gi = 5477606/ug = Mm 41785/len = 394/STRA = for | | | | | | |
| 16 | 99191_at | Cluster Incl AI844939 UI-M-BG0-ahv-g-09-0-UI s1 Mus musculus cDNA, 3 end/clone = UI-M-BG0-ahv-g-09-0-UI/ | 1615.8 | P | 1106.2 | P | 1030 | P |
| | | clone_end = 3/gb = AI844939/gi = 5488845/ug = Mm 41906/len = 413/STRA = for | | | | | | |
| 16 | 99617_at | Cluster Incl AI846299: UI-M-AK1-aez-f-12-0-UI s1 Mus musculus cDNA, 3 end/clone = UI-M-AK1-aez-f-12-0-UI/ | 1132.8 | P | 1063.1 | P | 1064.6 | P |
| | | clone_end = 3/gb = AI846299/gi = 5490205/ug = Mm 43040/len = 335/STRA = for | | | | | | |
| 16 | 100125_at | Cluster Incl U43918 Mus musculus proliferation-associated protein 1 mRNA, complete cds/cds = (266,1450)/gb = U43918/ | 516.7 | P | 359.4 | P | 445.4 | P |
| | | gi = 1167966/ug = Mm 4742/len = 1714/STRA = for | | | | | | |
| 16 | 100594_at | Cluster Incl AB008895: Mus musculus mRNA for mGpI1p, complete cds/cds = (140,1885)/gb = AB008895/gi = 2641975/ | 229.1 | P | 163.2 | P | 156 | P |
| | | ug = Mm. 6354/len = 3062/STRA = for | | | | | | |
| 16 | 100633_at | Cluster Incl AW124268: UI-M-BH2 1-aph-h-08-0-UI s1 Mus musculus cDNA, 3 end/clone = UI-M-BH2 1-aph-h-08-0-UI/ | 763.8 | P | 802.9 | P | 973.5 | P |
| | | clone_end = 3/gb = AW124268/gi = 6099798/ug = Mm 6696/len = 385/STRA = for | | | | | | |
| 16 | 101090_at | Cluster Incl L29454: Fibnllin 1/cds = (2379,10994)/gb = L29454/gi = 575509/ug = Mm.735/len = 11953/STRA = for | 1040.6 | P | 799.3 | P | 994.9 | P |
| 16 | 101585_at | Cluster Incl AF042491 Mus musculus putative membrane associated progesterone receptor component mRNA, complete | 763.2 | P | 676.2 | P | 666.4 | P |
| | | cds/cds = (13,600)/gb = AF042491/gi = 2801792/ug = Mm 9052/len = 1786/STRA = for | | | | | | |
| 16 | 101989_at | Cluster Incl AW125380 UI-M-BH2 3-aqh-b-06-0-UI.s1 Mus musculus cDNA, 3 end/clone = UI-M-BH2 3-aqh-b-06-0-UI/ | 2612.3 | P | 1833.7 | P | 2674.8 | P |
| | | clone_end = 3/gb = AW125380/gi = 6100910/ug = Mm 972/len = 269/STRA = for | | | | | | |
| 16 | 102002_at | Cluster Incl AI836406 UI-M-AP0-abi-g-05-0-UI s1 Mus musculus cDNA, 3 end/clone = UI-M-AP0-abi-g-05-0-UI/ | 423. 7 | P | 313.7 | P | 423.4 | P |
| | | clone_end = 3/gb = AI836406/gi = 5470619/ug = Mm 99026/len = 435/STRA = for | | | | | | |
| 16 | 102061_at | Cluster Incl AF100171: Mus musculus myelodysplasia/myeloid leukemia factor 1 (Mlf1) mRNA, complete cds/cds = (60,863)/ | 184.2 | P | 133 | P | 201.4 | P |
| | | gb = AF100171/gi = 4106525/ug = Mm.10414/len = 879/STRA = for | | | | | | |
| 16 | 102384_at | Cluster Incl AI842968 UI-M-AO1-aem-g-04-0-UI.s1 Mus musculus cDNA, 3 end/clone = UI-M-AO1-aem-g-04-0-UI/ | 154.5 | P | 143.9 | P | 107.8 | P |
| | | clone_end = 3/gb = AI842968/gi = 5477181/ug = Mm.12184/len = 467/STRA = for | | | | | | |
| 16 | 102794_at | Cluster Incl Z80112 Mus musculus lcr-1 gene/cds = (0,1073)/gb = Z80112/gi = 2632100/ug = Mm 1401/len = 1180/STRA = for | 50.5 | P | 92.9 | P | 66.1 | P |
| 16 | 102859_at | Cluster Incl AW121304: UI-M-BH2.3-aog-g-02-0-UI s1 Mus musculus cDNA, 3 end/clone = UI-M-BH2 3-aog-g-02-0-UI/ | 376.4 | P | 265.9 | P | 259.9 | P |
| | | clone_end = 3/gb = AW121304/gi = 6096637/ug = Mm 14992/len = 261/STRA = for | | | | | | |
| 16 | 103556_at | Cluster Incl AI840158 UI-M-AO0-acc-d-08-0-Ul.s1 Mus musculus cDNA, 3 end/clone = UI-M-AO0-aoc-d-08-0-UI/ | 1139.4 | P | 788.4 | P | 799.8 | P |
| | | clone_end = 3/gb = AI840158/gi = 5474371/ug = Mm 19081/len = 406/STRA = for | | | | | | |
| 16 | 103570_at | Cluster Incl AI315647 uj31c11 x1 Mus musculus cDNA, 3 end/clone = IMAGE-1921556/clone_end = 3/gb = AI315647/ | 490.6 | P | 193 | P | 157.6 | P |
| | | gi = 4030914/ug = Mm 19310/len = 467/STRA = for | | | | | | |
| 16 | 103592_at | Cluster Incl AB019374 Mus musculus mRNA for MEK5, complete cds/cds = (596,1942)/gb = AB019374/gi = 5360522/ | 276.8 | P | 142.5 | P | 246.2 | P |
| | | ug = Mm 19947/len = 2259/STRA = for | | | | | | |
| 16 | 103650_at | Cluster Incl AI846887 UI-M-AK1-aey-a-01-0-UI s1 Mus musculus cDNA, 3 end/clone = UI-M-AK1-aey-a-01-0-UI/ | 300.4 | P | 176.3 | P | 348.6 | P |
| | | clone_end = 3/gb = AI846887/gi = 5490793/ug = Mm 20414/len = 320/STRA = for | | | | | | |
| 16 | 103812_at | Cluster Incl AF047838: Mus musculus calcium-sensitive chlonde conductance protein-1 (mCLCA1) mRNA, complete cds/ | 297.7 | P | 166.8 | P | 171.7 | P |
| | | cds = (17,2725)/gb = AF047838/gi = 3925280/ug = Mm 20897/len = 3022/STRA = rev | | | | | | |
| 16 | 103829_at | Cluster Incl AF061529 Mus musculus rjs (rjs) mRNA, complete cds/cds = (119,14629)/gb = AF061529/gi = 3414808/ | 103.1 | P | 0.3 | P | 26.9 | P |
| | | ug = Mm 20929/len = 15264/STRA = for | | | | | | |
| 16 | 104345_at | Cluster Incl AF014461 Mus musculus EXO70 protein (Exo70) mRNA, complete cds/cds = (0,2093)/gb = AF014461/ | 163.7 | P | 120.4 | P | 318.9 | P |
| | | gi = 2352997/ug = Mm 22530/len = 2094/STRA = for | | | | | | |
| 16 | 104365_at | Cluster Incl AW046162 UI-M-BH1-akw-b-04-0-UI s1 Mus musculus cDNA, 3 end/clone = UI-M-BH1-akw-b-04-0-UI/ | 418.9 | P | 378.8 | P | 450 | P |
| | | clone_end = 3/gb = AW046162/gi = 5906691/ug = Mm 22615/len = 456/STRA = for | | | | | | |
| 16 | 104572_at | Cluster Incl AI852047: UI-M-BH0-aja-b-01-0-UI s1 Mus musculus cDNA, 3 end/clone = UI-M-BH0-aja-b-01-0-UI/ | 486.9 | P | 456.6 | P | 375.7 | P |
| | | clone_end = 3/gb = AI852047/gi = 5495953/ug = Mm 23910/len = 523/STRA = for | | | | | | |
| 16 | 104725_at | Cluster Incl AW060401 UI-M-BH1-anj-a-06-0-UI s1 Mus musculus cDNA, 3 end/clone = UI-M-BH1-anj-a-06-0-UI/ | 534.8 | P | 372.5 | P | 436.2 | P |
| | | clone_end = 3/gb = AW060401/gi = 6008152/ug = Mm 24574/len = 467/STRA = for | | | | | | |
| 16 | 104746_at | Cluster IncI AF040252 Mus musculus FK506-binding protein (FKBP23) mRNA, complete cds/cds = (56,712)/gb = AF040252/ | 314.5 | P | 195.8 | P | 233.6 | P |
| | | gi = 2827254/ug = Mm 24720/len = 866/STRA = for | | | | | | |
| 16 | 94331_at | Cluster Incl L47650 Signal transducer and activator of transcription 6/cds = (279,2792)/gb = L47650/gi = 1008876/ | 397.5 | P | 311.8 | P | 328.8 | P |
| | | ug = Mm 2489/len = 3213/STRA = for | | | | | | |
| 16 | 94348_f_at | Cluster Incl AW124044 UI-M-BH2.1-apn-d-10-0-UI s1 Mus musculus cDNA, 3 end/clone = UI-M-BH2 1-apn-d-10-0-UI/ | 654.5 | P | 397.4 | P | 390.8 | P |
| | | clone_end = 3/gb = AW124044/gi = 6099574/ug = Mm.25169/len = 408/STRA = for | | | | | | |
| 16 | 94392_f_at | Cluster Incl U22516: Angiogenin/cds = (0,437)/gb = U22516/gi = 726325/ug = Mm 2566/len = 438/STRA = for | 109.8 | P | 148.3 | P | 151.4 | P |
| 16 | 94422_at | Cluster Incl AI842938 UI-M-AO1-aem-d-04-0-UI.s1 Mus musculus cDNA, 3 end/clone = UI-M-AO1-aem-d-04-0-UI/ | 156.6 | P | 131.9 | P | 134.3 | P |
| | | clone_end = 3/gb = AI842938/gi = 5477151/ug = Mm.26242/len = 303/STRA = for | | | | | | |
| 16 | 94930_at | Cluster Incl L07803: Thrombospondin 2/cds = (218,3736)/gb = L07803/gi = 340421/ug = Mm 26688/len = 4108/STRA = for | 430 | P | 255.4 | P | 564.9 | P |
| 16 | 94976_at | Cluster Incl AI845877 Ul-M-AO1-aeg-g-12-0-UI s1 Mus musculus cDNA, 3 end/clone = UI-M-AO1-aog-g-12-0-UI/ | 441.1 | P | 355.6 | P | 649.1 | P |
| | | clone_end = 3/gb = AI845877/gi = 5489783/ug = Mm.27259/len = 409/STRA = rev | | | | | | |
| 16 | 94995_at | Cluster Incl AI854331 UI-M-BG1-aic-b-07-0-UI s1 Mus musculus cDNA, 3 end/clone = UI-M-BG1-aic-b-07-0-UI/ | 1006.3 | P | 889.9 | P | 688.5 | P |
| | | clone_end = 3/gb = AI854331/gi = 5498237/ug = Mm.27337/len = 404/STRA = rev | | | | | | |
| 16 | 95014_at | Cluster Incl AA794247: vu65b08 r1 Mus musculus cDNA, 5 end/clone = IMAGE-1196247/clone_end = 5/gb = AA794247/ | 729.2 | P | 516.3 | P | 450 | P |
| | | gi = 2857202/ug = Mm.27445/len = 496/STRA = for | | | | | | |
| 16 | 95529_at | Cluster Incl U58884 Mus musculus SH3-containing protein SH3P7 mRNA, complete cds similar to Human Drebrin/ | 751 | P | 573.1 | P | 542.8 | P |
| | | cds = (30,1331)/gb = U58884/gi = 1407654/ug = Mm 27622/len = 2291/STRA = rev | | | | | | |
| 16 | 95565_at | Cluster Incl AI852873 UI-M-BH0-ajk-f-03-0-Ul.s1 Mus musculus cDNA, 3 end/clone = UI-M-BH0-ajk-f-03-0-UI/clone_end = 3/ | 585.7 | P | 564.3 | P | 503.5 | P |
| | | gb = AI852873/gi = 5496779/ug = Mm 27787/len = 327/STRA = for | | | | | | |
| 16 | 95590_at | Cluster Incl AA615951: vo95c11.r1 Mus musculus cDNA, 5 end/clone = IMAGE-1066868/clone_end = 5/gb = AA615951/ | 202.5 | P | 194.5 | P | 202.7 | P |
| | | gi = 2503187/ug = Mm 27890/len = 621/STRA = for | | | | | | |
| 16 | 95597_at | Cluster Incl M34141 Prostaglandin-endoperoxide synthase 1/cds = (36,1844)/gb = M34141/gi = 200302/ug = Mm.2792/ | 202.2 | P | 175.1 | P | 240 | P |
| | | len = 2757/STRA = for | | | | | | |
| 16 | 95617_at | Cluster Incl U36799 Retinoblastoma-like 2/cds = (19,3396)/gb = U36799/gi = 1045519/ug = Mm 28027/len = 4806/STRA = for | 103.5 | P | 78.7 | P | 106.9 | P |
| 16 | 95622_at | Cluster Incl AW123907 UI-M-BH2 1-app-h-01-0-UI.s1 Mus musculus cDNA, 3 end/clone = UI-M-BH2.1-app-h-01-0-UI/ | 408.6 | P | 273.3 | P | 350.9 | P |
| | | clone_end = 3/gb = AW123907/gi = 6099437/ug = Mm 28047/len = 326/STRA = for | | | | | | |
| 16 | 95630_at | Cluster Incl AW122852 UI-M-BH2 1-aoz-g-07-0-UI s1 Mus musculus cDNA, 3 end/clone = UI-M-BH2 1-aoz-g-07-0-UI/ | 694.1 | P | 396.3 | P | 462.9 | P |
| | | clone_end = 3/gb = AW122852/gi = 6098382/ug = Mm 28080/len = 351/STRA = rev | | | | | | |
| 16 | 96127_at | Cluster Incl AW048730: UI-M-BH1-amy-e-02-0-UI s1 Mus musculus cDNA, 3 end/clone = UI-M-BH1-amy-e-02-0-UI/ | 868.8 | P | 735.6 | P | 781.5 | P |
| | | clone_end = 3/gb = AW048730/gi = 5909259/ug = Mm 28119/len = 536/STRA = for | | | | | | |
| 16 | 96765_at | Cluster Incl AW120874: UI-M-BH2.3-aoa-h-02-0-UI.s1 Mus musculus cDNA, 3 end/clone = UI-M-BH2.3-aoa-h-02-0-UI/ | 1673 | P | 1220.6 | P | 1320.5 | P |
| | | clone_end = 3/gb = AW120874/gi = 6096207/ug = Mm.29009/len = 298/STRA = for | | | | | | |
| 16 | 96802_at | Cluster Incl AW124656: UI-M-BH2 1-apw-e-04-0-U1.s1 Mus musculus cDNA, 3 end/clone = UI-M-BH2.1-apw-e-04-0-UI/ | 117.2 | P | 35.1 | P | 103.9 | P |
| | | clone_end = 3/gb = AW124656/gi = 6100186/ug = Mm.29193/len = 368/STRA = for | | | | | | |
| 16 | 97977_at | Cluster Incl AA645293 vs81a07.r1 Mus musculus cDNA, 5 end/clone = IMAGE-1152660/clone_end = 5/gb = AA645293/ | 544.5 | P | 439.2 | P | 495.5 | P |
| | | gi = 2571722/ug = Mm 3116/len = 276/STRA = for | | | | | | |
| 16 | 98468_r_at | Cluster Incl AI316859: ul35b03 y1 Mus musculus cDNA, 5 end/clone = IMAGE-1853261/clone_end = 5/gb = AI316659/ | 263.4 | P | 283.8 | P | 189.5 | P |
| | | gi = 4032126/ug = Mm 34879/len = 500/STRA = for | | | | | | |
| 16 | 98511_at | Cluster Incl L17076 HnRNP-associated with lethal yellow/cds = UNKNOWN/gb = L17076/gi = 348995/ug = Mm 3591/ | 474.6 | P | 560.2 | P | 534.9 | P |
| | | len = 1517/STRA = for | | | | | | |
| 16 | 99010_at | Cluster Incl AB024538 Mus musculus Islr(immunoglobulin superfamily containing leucine-nch repeat) mRNA, complete cds/ | 908.3 | P | 162.5 | P | 278.9 | P |
| | | cds = (89,1375)/gb = AB024538/gi = 6172374/ug = Mm 38426/len = 2038/STRA = for | | | | | | |
| 16 | 99029_at | Cluster Incl AF077950: Mus musculus protein inhibitor of activated STAT protein PIAS1 mRNA, complete cds/cds = (0,1955)/ | 341.9 | P | 261.8 | P | 223.2 | P |
| | | gb = AF077950/gi = 3643104/ug = Mm 38869/len = 1956/STRA = for | | | | | | |
| 16 | 99067_at | Cluster Incl X59846 M. musculus GAS 6 mRNA associated with growth-arrest/cds = (167,2188)/gb = X59846/gi = 407060/ | 363.2 | P | 212.4 | P | 325.8 | P |
| | | ug = Mm 3982/len = 2573/STRA = rev | | | | | | |
| 16 | 99476_at | Cluster Incl AJ131395: Mus musculus mRNA for collagen type XIV, partial/cds = (0,380)/gb = AJ131395/gi = 5042293/ | 591.5 | P | 462.8 | P | 576.9 | P |
| | | ug = Mm.41332/len = 1171/STRA = for | | | | | | |
| 16 | 99518_at | Cluster Incl AW121179 UI-M-BH2 3-aok-a-06-0-UI s1 Mus musculus cDNA, 3 end/clone = UI-M-BH2 3-aok-a-06-0-UI/ | 583.5 | P | 702.6 | P | 800 | P |
| | | clone_end = 3/gb = AW121179/gi = 6096512/ug = Mm 41931/len = 479/STRA = for | | | | | | |
| 16 | 99940_at | Cluster Incl AW212667 uo10c01 x1 Mus musculus cDNA, 3 end/clone = IMAGE-2599104/clone_end = 3/gb = AW212667/ | 192.2 | P | 145.3 | P | 144 | P |
| | | gi = 6518780/ug = Mm 435/len = 464/STRA = for | | | | | | |
| 16 | 99947_at | Cluster Incl U55861 Mus musculus RNA binding protein TIAR mRNA, complete cds/cds = (133,1311)/gb = U55861/ | 393.7 | P | 315.8 | P | 404.2 | P |
| | | gi = 1592562/ug = Mm 4363/len = 1543/STRA = for | | | | | | |
| 16 | 100443_at | Cluster Incl AF031467 Branched chain aminotransferase 2, mitochondrial/cds = (135,1196)/gb = AF031467/gi = 3298578/ | 264.7 | P | 151.9 | P | 284.4 | P |
| | | ug = Mm 4811/len = 1200/STRA = for | | | | | | |
| 16 | 100477_at | Cluster Incl M32486 Mouse 19.5 mRNA, complete cds/cds = (54,875)/gb = M32486/gi = 191495/ug = Mm 4992/len = 1183/ | 1828.2 | P | 1654.7 | P | 1496.9 | P |
| | | STRA = for | | | | | | |
| 16 | 101416_f_at | Cluster Incl L76155 Mus musculus Bat-4 gene, complete cds/cds = (40,1158)/gb = L76155/gi = 1196643/ug = Mm 7405/ | 755.2 | P | 593.5 | P | 587.2 | P |
| | | len = 1400/STRA = for | | | | | | |
| 16 | 104408_s_at | Cluster Incl L35032 SRY-box containing gene 18/cds = (88,1221)/gb = L35032/gi = 1663531/ug = Mm 2878/len = 1585/ | 400.8 | P | 320.9 | P | 313.4 | P |
| | | STRA = for | | | | | | |
| 16 | 104445_at | Cluster Incl AW046694 UI-M-BH1-alm-d-07-0-UI s1 Mus musculus cDNA, 3 end/clone = UI-M-BH1-alm-d-07-0-UI/ | 879.3 | P | 875 | P | 743 | P |
| | | clone_end = 3/gb = AW046694/gi = 5907223/ug = Mm 2935/len = 253/STRA = for | | | | | | |
| 16 | 92207_at | Cluster Incl U08210 Elastin/cds = (0,2582)/gb = U08210/gi = 473273/ug = Mm 34170/len = 2651/STRA = for | 754.4 | P | 615.4 | P | 575.3 | P |
| 16 | 92224_at | Cluster Incl X79199 Tetranectin (plasminogen-binding protein)/cds = (73,681)/gb = X79199/gi = 671501/ug = Mm 34588/ | 417.9 | P | 337.7 | P | 618.7 | P |
| | | len = 988/STRA = rev | | | | | | |
| 16 | 92375_at | Cluster Incl AW211207 ut29a03 y1 Mus musculus cDNA, 5 end/clone = IMAGE-2099692/clone_end = 5/gb = AW211207/ | 245 | P | 184.4 | P | 209.3 | P |
| | | gi = 6517147/ug = Mm 4002/len = 531/STRA = for | | | | | | |
| 16 | 93443_at | Cluster Incl AW212271 uo84g08 x1 Mus musculus cDNA, 3 end/clone = IMAGE-2649278/clone_end = 3/gb = AW212271/ | 352.6 | P | 297.4 | P | 293.1 | P |
| | | gi = 6515306/ug = Mm 6671/len = 420/STRA = for | | | | | | |
| 16 | 93472_at | Cluster Incl AA796989: vt01a04 r1 Mus musculus cDNA, 5 end/clone = IMAGE-1154478/clone_end = 5/gb = AA796989/ | 2023.2 | P | 1605.5 | P | 1341.3 | P |
| | | gi = 2859944/ug = Mm.7049/len = 428/STRA = for | | | | | | |
| 16 | 93485_at | Cluster Incl AI844911 UI-M-BG0-ahv-d-10-0-UI s1 Mus musculus cDNA, 3 end/clone = UI-M-BG0-ahv-d-10-0-UI/ | 177.9 | P | 139.8 | P | 193.4 | P |
| | | clone_end = 3/gb = AI844911/gi = 5488617/ug = Mm 7172/len = 399/STRA = for | | | | | | |
| 16 | 93615_at | Cluster Incl AF020200 Mus musculus transcription factor PBX3b (PBX3b) mRNA, complete cds/cds = (118,1173)/ | 158.2 | P | 132.6 | P | 189.2 | P |
| | | gb = AF020200/gi = 2432016/ug = Mm.7331/len = 2467/STRA = for | | | | | | |
| 16 | 93874_s_at | Cluster Incl U69491: lnterleukin 11 receptor, alpha chain 2/cds = (301,1599)/gb = U69491/gi = 1916003/ug = Mm 88405/ | 1013.9 | P | 472.6 | P | 610.2 | P |
| | | len = 1975/STRA = for | | | | | | |
| 16 | 99812_at | Cluster Incl X92523 Calpain 3/cds = (0,2465)/gb = X92523/gi = 1246778/ug = Mm 57235/len = 2466/STRA = for | 238.2 | P | 240.3 | P | 170.8 | P |
| 16 | 94642_at | Cluster Incl U07951 Mus musculus BALB/c GDP-dissociation inhibitor (GDI-2) mRNA, complete cds/cds = (289,1626)/ | 154.8 | P | 163.5 | P | 162.6 | P |
| | | gb = U07951/gi = 1486479/ug = Mm 8070/len = 1676/STRA = for | | | | | | |
| 16 | 93835_at | Cluster Incl AI843222: UI-M-AK1-aet-g-05-0-UI s1 Mus musculus cDNA, 3 end/clone = UI-M-AK1-ael-g-05-0-UI/ | 191.8 | P | 102.8 | A | 128.1 | P |
| | | clone_end = 3/gb = AI843222/gi = 5477435/ug = Mm.21580/len = 373/STRA = for | | | | | | |
| 16 | 94079_at | Cluster Incl X61452 M musculus mRNA for H5 clone/cds = (35,1471)/gb = X61452/gi = 51202/ug = Mm 2214/len = 1649/ | 174.6 | P | 154 | P | 136.4 | P |
| | | STRA = for | | | | | | |
| 16 | 94275_at | Cluster Incl U34691 Uroporphynnogen decarboxylase/cds = (281,1384)/gb = U34691/gi = 1498642/ug = Mm 22494/len = 1454/ | 175.4 | A | 211.4 | P | 215.3 | P |
| | | STRA = for | | | | | | |
| 16 | 94304_at | Cluster Incl X13460: Annexin VI, p68/cds = (33,2054)/gb = X13460/gi = 53580/ug = Mm 22619/len = 2402/STRA = for | 736.5 | P | 769.3 | A | 693.8 | P |
| 16 | 97835_at | Cluster Incl AI842828 UI-M-AP1-agi-h-10-0-UI s1 Mus musculus cDNA, 3 end/clone = UI-M-Ap1-agi-h-10-0-UI/ | 553.6 | P | 398.4 | A | 801.4 | P |
| | | clone_end = 3/gb = AI842828/gi = 5477041/ug = Mm28231/len = 175/STRA = for | | | | | | |
| 16 | 98923_at | Cluster Incl AI852608 UI-M-BH0-alu-d-07-0-UI s1 Mus musculus cDNA, 3 end/clone = UI-M-BH0-alu-d-07-0-UI/ | 78.2 | P | 34.7 | A | 169.6 | P |
| | | clone_end = 3/gb = AI852608/gi = 5496514/ug = Mm 28630/len = 273/STRA = for | | | | | | |
| 16 | 94459_at | Cluster Incl AA681764 v161b04 r1 Mus musculus cDNA, 5 end/clone = IMAGE-1167535/clone_end = 5/gb = AA681764/ | 132.2 | P | 50.4 | A | 158.5 | P |
| | | gi = 2663904/ug = Mm.28818/len = 441/STRA = for | | | | | | |
| 16 | 96296_at | Cluster Incl AI843685 UI-M-AK1-aer-c-07-0-UI s1 Mus musculus cDNA, 3 end/clone = UI-M-AK1-aer-c-07-0-UI/ | 366.4 | A | 311.6 | P | 365.1 | P |
| | | clone_end = 3/gb = AI843685/gi = 5477898/ug = Mm.29904/len = 305/STRA = for | | | | | | |
| 16 | 99630_at | Cluster Incl AW060257 UI-M-BH1-amv-f-08-0-UI.s1 Mus musculus cDNA, 3 end/clone = UI-M-BH1-amv-f-08-0-UI/ | 155.7 | P | 91.5 | P | 101.1 | A |
| | | clone_end = 3/gb = AW060257/gi = 6008048/ug = Mm.43397/1en = 434/STRA = for | | | | | | |
| 16 | 101036_at | Cluster Incl AW047047 UI-M-BH1-alr-c-02-0-UI s1 Mus musculus cDNA, 3 end/clone = UI-M-BH1-alr-c-02-0-UI/ | 560.4 | P | 352.9 | A | 489.5 | P |
| | | clone_end = 3/gb = AW047047/gi = 5907576/ug = Mm.6932/len = 334/STRA = for | | | | | | |
| 16 | 103030_at | Cluster Incl L31397: Dynamin/cds = (73,2658)/gb = L31397/gi = 487856/ug = Mm 16080/len = 3924/STRA = rev | 393.9 | P | 201.1 | P | 223.3 | P |
| 16 | 104610_at | Cluster Incl AW050015: UI-M-BH1-amm-a-01-0-UI.s1 Mus musculus cDNA, 3 end/clone = UI-M-BH1-amm-a-01-0-UI/ | 136.3 | P | 169 | P | 145.5 | P |
| | | clone_end = 3/gb = AW050015/gi = 5910544/ug = Mm.2415/len = 503/STRA = rev | | | | | | |
| 16 | 104616_g_at | Cluster Incl M96265: Mus musculus galactose-1-phosphate uridyl transferase (GALT) mRNA, complete cds/cds = (131,1312)/ | 116.7 | P | 42.3 | A | 66.6 | P |
| | | gb = M96265/gi = 193421/ug = Mm 2420/len = 1373/STRA = for | | | | | | |
| 16 | 94428_at | Cluster Incl AI842668 UI-M-AO1-aeo-a-05-0-UI s1 Mus musculus cDNA, 3 end/clone = UI-M-AO1-aeo-a-05-0-UI/ | 231.6 | P | 139.5 | P | 274.2 | P |
| | | clone_end = 3/gb = AI842668/gi = 5476881/ug = Mm 2644/len = 378/STRA = for | | | | | | |
| 16 | 95537_at | Cluster Incl AB019577 Mus musculus mRNA for UNC-51-like kinase (ULK) 2, complete cds/cds = (41,3154)/gb = AB019577/ | 93.3 | P | 69.6 | A | 30.2 | P |
| | | gi = 4760560/ug = Mm 27664/len = 5275/STRA = for | | | | | | |
| 16 | 96728_at | Cluster Incl AI844151 UI-M-AL1-ahj-f-07-0-UI.s1 Mus musculus cDNA, 3 end/clone = UI-M-AL1-ahj-f-07-0-UI/clone_end = 3/ | 241.4 | P | 228.9 | P | 114.2 | P |
| | | gb = AI844151/gi = 5488057/ug = Mm 28850/len = 317/STRA = rev | | | | | | |
| 16 | 99929_at | Cluster Incl U72141: Exostoses (multiple) 2/cds = (191,2347)/gb = U72141/gi = 1619953/ug = Mm 4338/len = 2864/STRA = for | 275.5 | P | 305.8 | P | 416.8 | P |
| 16 | 99992_at | Cluster Incl AI286698 ub84b09 r1 Mus musculus cDNA, 5 end/clone = IMAGE-1395161/clone_end = 5/gb = AI286698/ | 221.8 | P | 192.6 | P | 225.2 | P |
| | | gi = 3926451/ug = Mm.4481/len = 200/STRA = for | | | | | | |
| 16 | 100468_g_at | Cluster Incl X57667 M musculus LYL gene (clone L6)/cds = (465,1301)/gb = X57687/gi = 52965/ug = Mm 4925/len = 1686/ | 72.9 | P | 66.5 | P | 89.6 | P |
| | | STRA = for | | | | | | |
| 16 | 101446_at | Cluster Incl AF004428, Turnor protein D52-like 1/cds = (191,805)/gb = AF004428/gi = 2895080/ug = Mm.7821/len = 1267/ | 121.1 | P | 90.1 | P | 96.4 | P |
| | | STRA = for | | | | | | |
| 16 | 102720_at | Cluster Incl X71426 Endothelial-specific receptor tyrosine kinase/cds = (86 3454)/gb = X71426/gi = 296612/ug = Mm 14313/ | 121.9 | P | 126.7 | P | 126.2 | P |
| | | len = 3490/STRA = for | | | | | | |
| 16 | 103536_at | Cluster Incl AB017270 Mus musculus TMEFF2 mRNA, complete cds/cds = (388,1512)/gb = AB017270/gi = 6518464/ | 112.4 | P | 0.1 | P | 109.1 | P |
| | | ug = Mm 23304/len = 1740/STRA = for | | | | | | |
| 16 | 103922_f_at | Cluster Incl AI839690 UI-M-AN0-acp-e-04-0-UI s1 Mus musculus cDNA, 3 end/clone = UI-M-AN0-acp-e-04-0-UI/ | 158.7 | P | 117.6 | A | 123.7 | P |
| | | clone_end = 3/gb = AI839690/gi = 5473903/ug = Mm 25034/len = 250/STRA = rev | | | | | | |
| 16 | 104680_at | Cluster Incl AJ250489 Mus musculus mRNA for receptor activity modifying protein 1 (Ramp1 gene)/cds = (59,505)/ | 380.1 | P | 183.6 | A | 286.2 | P |
| | | gb = AJ250489/gi = 6119624/ug = Mm 3272/len = 844/STRA = for | | | | | | |
| 16 | 92366_at | Cluster Incl U12147.Laminin, alpha 2/cds = (54,9374)/gb = U12147/gi = 699109/ug = Mm 3987/len = 9511/STRA = for | 451.4 | P | 315.2 | A | 304.6 | P |
| 16 | 92397_at | Cluster Incl AW123016 UI-M-BH2 1-apb-e-06-0-UI s1 Mus musculus cDNA, 3 end/clone = UI-M-BH2.1-apb-e-06-0-UI/ | 445.1 | P | 454.5 | P | 478.5 | P |
| | | clone_end = 3/gb = AW123016/gi = 6098546/ug = Mm 4077/len = 473/STRA = rev | | | | | | |
| 16 | 92739_at | Cluster Incl L28819 Involucnn/cds = (0,1403)/gb = L28819/gi = 454418/ug = Mm.46792/len = 1404/STRA = for | 228.5 | P | 168 | A | 231 | P |
| 16 | 92894_s_at | Cluster Incl D90174 Mouse mRNA for NFI-B protein, clone NFI-B3/cds = (237,1742)/gb = D90174/gi = 220520/ug = Mm 4771/ | 128.2 | P | 213.3 | P | 148.5 | P |
| | | len = 1859/STRA = for | | | | | | |
| 16 | 93421_at | Cluster Incl AF033655 PFTAIRE protein kinase 1/cds = (85,1494)/gb = AF033655/gi = 2645809/ug = Mm 6456/len = 4529/ | 114.3 | P | 96.9 | P | 133.2 | A |
| | | STRA = for | | | | | | |
| 16 | 97155_at | Cluster Incl U84005 Myostatin/cds = (103,1233)/gb = U84005/gi = 2065554/ug = Mm.3514/len = 2676/STRA = for | 355.8 | P | 266.3 | A | 271.8 | P |
| 16 | 97874_at | Cluster Incl AI853863 UI-M-BH0-ajr-f-07-0-UI.s1 Mus musculus cDNA, 3 end/clone = UI-M-BH0-ajr-f-07-0-UI/clone_end = 3/ | 277 | P | 255.8 | A | 216.1 | P |
| | | gb = AI853863/gi = 5497769/ug = Mm 28349/len = 440/STRA = for | | | | | | |
| 16 | 95693_at | Cluster Incl U51167 Isocitrate dehydrogenase 2/cds = (23,1594)/gb = U51167/gi = 1236983/ug = Mm 2966/len = 1863/ | 838.7 | P | 331.6 | A | 590.3 | P |
| | | STRA = for | | | | | | |
| 16 | 98144_f_at | Cluster Incl AI844321 UI-M-AL1-ahm-f-07-0-UI s1 Mus musculus cDNA, 3 end/clone = UI-M-AL1-ahm-f-07-0-UI/ | 509.1 | P | 290.5 | A | 368.8 | P |
| | | clone_end = 3/gb = AI844321/gi = 5488227/ug = Mm 35650/len = 381/STRA = for | | | | | | |
| 16 | 102029_at | Cluster Incl AF017111 Interleukin 16/cds = (168,2042)/gb = AF017111/gi = 3127042/ug = Mm 10137/len = 2876/STRA = rev | 138.4 | P | 61.3 | P | 92.2 | A |
| 16 | 103637_at | Cluster Incl AJ223966 N-acetyl galactosaminidase, alpha/cds = (0,1247)/gb = AJ223966/gi = 3327015/ug = Mm 20325/ | 317.2 | P | 153.5 | A | 318.5 | P |
| | | len = 1248/STRA = for | | | | | | |
| 16 | 94417_at | Cluster Incl AI852949 UI-M-BH0-aiz-e-01-0-UI s1 Mus musculus cDNA, 3 end/clone = UI-M-BH0-aiz-e-01-0-UI/ | 117 | P | 64.9 | P | 89.8 | P |
| | | clone_end = 3/gb = AI852949/gi = 5496855/ug = Mm 26168/len = 338/STRA = for | | | | | | |
| 16 | 96176_at | Cluster Incl AI847916 UI-M-AI1-afl-c-02-0-UI s1 Mus musculus cDNA, 3 end/clone = UI-M-AI1-afl-c-02-0-UI/clone_end = 3/ | 327.5 | P | 356 | A | 350.4 | P |
| | | gb = AI847916/gi = 5491822/ug = Mm 28409/len = 447/STRA = for | | | | | | |
| 16 | 98449_at | Cluster Incl AB014721 Mus musculus mRNA for mSART-1(806), complete cds/cds = (34,2454)/gb = AB014721/gi = 4126468/ | 51 | P | 60 | P | 35.4 | P |
| | | ug = Mm 34562/len = 2513/STRA = rev | | | | | | |
| 16 | 100943_at | Cluster Incl U75215 Mus musculus neutral amino acid transporter mASCT1 mRNA, complete cds/cds = = (111,1709)/ | 202.3 | P | 212.3 | A | 239.2 | A |
| | | gb = U75215/gi = 2459560/ug = Mm 6379/len = 3787/STRA = for | | | | | | |
| 16 | 102227_g_at | Cluster Incl AF017639 Mus musculus carboxypeptidase X2 mRNA, complete cds/cds = (129,2423)/gb = AF017639/ | 118.1 | P | 86.4 | P | 137.2 | P |
| | | gi = 2921091/ug = Mm 10233/len = 2490/STRA = for | | | | | | |
| 16 | 103502_at | Cluster Incl AA866768 ud07g06 r1 Mus musculus cDNA, 5 end/clone = IMAGE-1434490/clone_end = 5/gb = AA866768/ | 96.2 | P | 101.8 | P | 118.1 | P |
| | | gi = 2962213/ug = Mm 22367/len = 376/STRA = for | | | | | | |
| 16 | 92450_at | Cluster Incl AF047339: Solute camer family 12, member 4/cds = (0,3257)/gb = AF047339/gi = 3452286/ug = Mm 4190/ | 332.1 | P | 323.5 | P | 353.6 | P |
| | | len = 3775/STRA = for | | | | | | |
| 16 | 97784_at | Cluster Incl Y14695 Mus musculus mRNA for epidermis-type lipoxygenase-3/cds = (26,2161)/gb = Y14695/gi = 5304927/ | 524.3 | P | 374.3 | P | 215.9 | P |
| | | ug = Mm 41989/len = 2536/STRA = for | | | | | | |
| 16 | 100743_at | Cluster Incl AA407663 EST01744 Mus musculus cDNA, 3 end/clone = C0013E09/clone_end = 3/gb = AA407663/ | 173.8 | P | 93.7 | P | 144 | P |
| | | gi = 2066659/ug = Mm 3640/len = 265/STRA = rev | | | | | | |
| 16 | 94504_at | Cluster Incl AI838655 UI-M-AO0-aca-c-08-0-UI.s1 Mus musculus cDNA, 3 end/clone = UI-M-AO0-aca-c-08-0-UI/ | 376.4 | P | 106.5 | A | 325.7 | P |
| | | clone_end = 3/gb = AI838655/gi = 5472868/ug = Mm 28955/len = 343/STRA = rev | | | | | | |
| 16 | 95642_at | Cluster Incl AI842813 UI-M-AP1-agi-g-02-0-UI.s1 Mus musculus cDNA, 3 end/clone = UI-M-AP1-agi-g-02-0-UI/ | 214.2 | P | 231.2 | P | 657.7 | A |
| | | clone_end = 3/gb = AI842813/gi = 5477026/ug = Mm 29492/len = 403/STRA = for | | | | | | |
| 16 | 99156_at | Cluster Incl AI853370 UI-M-BH0-air-e-04-0-UI s1 Mus musculus cDNA, 3 end/clone = UI-M-BH0-air-e-04-0-UI/ | 172.4 | P | −18.6 | A | 75.4 | M |
| | | clone_end = 3/gb = AI853370/gi = 5497276/ug = Mm.41617/len = 415/STRA = for | | | | | | |
| 16 | 101571_g_at | Cluster Incl X76066 M musculus gene for insulin like growth factor binding protein 4/cds = (0,764)/gb = X76066/gi = 416032/ | 779.7 | P | 539.7 | A | 713.8 | P |
| | | ug = Mm 89842/len = 765/STRA = for | | | | | | |
| 16 | 102343_at | Cluster Incl AF110520: Mus musculus major histocompatibility complex region NG27, NG28, RPS28, NADH | 352.7 | M | 233.2 | M | 510.5 | P |
| | | oxidoreductase, NG29, KIFC1, Fas-binding protein, BING1, tapasin, RaIGDS-like, KE2, BING4, beta 1,3-galactosyl transferase, and RPS18 genes, complete cds; S/STRA = for | | | | | | |
| 16 | 103017_at | Cluster Incl AI060729 ub42h10 r1 Mus musculus cDNA, 5 end/clone = IMAGE-1380451/clone_end = 5/gb = AI060729/ | 131.4 | M | 50.8 | P | 107.9 | P |
| | | gi = 3336152/ug = Mm 1565/len = 457/STRA = for | | | | | | |
| 16 | 97964_at | Cluster Incl AW122851: UI-M-BH2 1-aoz-g-06-0-UI s1 Mus musculus cDNA, 3 end/clone = UI-M-BH2 1-aoz-g-06-0-UI/ | 131 | P | 47.7 | A | 129 | P |
| | | clone_end = 3/gb = AW122851/gi = 6098381/ug = Mm 30729/len = 462/STRA = for | | | | | | |
| 16 | 100408_at | Cluster Incl AA839465-vw50d06 r1 Mus musculus cDNA, 5 end/clone = IMAGE-1247243/clone_end = 5/gb = AA839465/ | 119.1 | P | 95.7 | P | 59.6 | A |
| | | gi = 2915560/ug = Mm 46563/len = 437/STRA = for | | | | | | |
| 16 | 100991_at | Cluster Incl AI852849 UI-M-BH0-ajk-d-01-0-UI s1 Mus musculus cDNA, 3 end/clone = UI-M-BH0-ajk-d-01-0-UI/ | 301.9 | P | 294.3 | P | 375.7 | P |
| | | clone_end = 3/gb = AI852849/gi = 5496755/ug = Mm 6818/len = 348/STRA = for | | | | | | |
| 16 | 101901_at | Cluster Incl AB024713 Mus musculus mRNA for Supl15h, complete cds/cds = (6,749)/gb = AB024713/gi = 5103141/ | 246 | A | 273.8 | A | 257 | A |
| | | ug = Mm 89579/len = 1249/STRA = for | | | | | | |
| 16 | 102969_at | Cluster Incl AI847369 UI-M-AN1-afh-g-04-0-UI s1 Mus musculus cDNA, 3 end/clone = UI-M-AN1-afh-g-04-0-UI/ | 162.9 | P | 130.3 | A | 112.3 | P |
| | | clone_end = 3/gb = AI847369/gi = 5491240/ug = Mm 183/len = 436/STRA = for | | | | | | |
| 16 | 103273_s_at | Cluster Incl AF037312 untitled/cds = (0,2051)/gb = AF037312/gi = 3851489/ug = Mm 20868/len = 2147/STRA = for | 154 | P | 151.8 | P | 123.3 | A |
| 16 | 96577_i_at | Cluster Incl AI853019 UI-M-BH0-ajn-d-04-0-UI s1 Mus musculus cDNA, 3 end/clone = UI-M-BH0-ajn-d-04-0-UI/ | 138.9 | | 130.2 | P | 167.2 | P |
| | | clone_end = 3/gb = AI853019/gi = 5496925/ug = Mm 3021/len = 250/STRA = for | | | | | | |
| 16 | 98859_at | Cluster Incl M99054 Acid phosphatase 5, tartrate resistant/cds = (132,1115)/gb = M99054/gi = 553868/ug = Mm.5119/ | 210.6 | A | 175.7 | A | 272.1 | P |
| | | len = 1386/STRA = for | | | | | | |
| 16 | 101703_at | Cluster Incl V00716 Mouse pseudogene for alpha-globin without intervening sequences (the normal alpha-globin gene has | 2346.7 | P | 992.2 | P | 2192.4 | P |
| | | two introns)/cds = (32,436)/gb = V00716/gi = 49902/ug = Mm 41983/len = 542/STRA = for | | | | | | |
| 16 | 92558_at | Cluster Incl M84487 Vascular cell adhesion molecule 1/cds = (57,2276)/gb = M84487/gi = 202345/ug = Mm 1021/len = 3014/ | 96.3 | P | 43.5 | P | 73.7 | A |
| | | STRA = for | | | | | | |
| 16 | 94861_at | Cluster Incl AA624073 vq71f12 s1 Mus musculus cDNA, 5 end/clone = IMAGE-1107791/clone_end = 5/gb = AA624073/ | 151 | P | 112.9 | M | 125.7 | P |
| | | gi = 2527949/ug = Mm.23682/len = 342/STRA = for | | | | | | |
| 16 | 98005_at | Cluster Incl AW125442 UI-M-BH2 3-aqh-g-12-0-UI s1 Mus musculus cDNA, 3 end/clone = UI-M-BH2 3-aqh-g-12-0-UI/ | 159.9 | P | 74.9 | A | 122.9 | P |
| | | clone_end = 3/gb = AW125442/gi = 6100972/ug = Mm 3193/len = 487/STRA = for | | | | | | |
| 16 | 101844_at | Cluster Incl U94700: Peroxisomal sarcosine oxidase/cds = (4,1176)/gb = U94700/gi = 2801410/ug = Mm 8543/len = 1686/ | 338.1 | P | 214.6 | P | 284.5 | A |
| | | STRA = for | | | | | | |
| 16 | 102684_at | Cluster Incl U82375 Mus musculus MSG2alpha, beta, gamma, delta and epsilon salivary protein (Vcs2) genes, complete | 15.9 | A | 27.8 | P | 23.4 | P |
| | | cds/cds = (154,585)/gb = U82375/gi = 2289098/ug = Mm 1397/len = 743/STRA = for | | | | | | |
| 16 | 92971_at | Cluster Incl AW125849 UI-M-BH2.2-aqk-d-08-0-UI.s1 Mus musculus cDNA, 3 end/clone = UI-M-BH2 2-aqk-d-08-0-UI/ | 152.7 | P | 90.4 | A | 127.9 | A |
| | | clone_end = 3/gb = AW125849/gi = 6101379/ug = Mm.5002/len = 468/STRA = rev | | | | | | |
| 16 | 93463_at | Cluster Incl AW122517 UI-M-BH2.2-aox-a-10-0-UI s1 Mus musculus cDNA, 3 end/clone = UI-M-BH2 2-aox-a-10-0-UI/ | −62.1 | P | −182.7 | P | −93.6 | A |
| | | clone_end = 3/gb = AW122517/gi = 6098041/ug = Mm 69024/len = 439/STRA = rev | | | | | | |
| 16 | 100634_at | Cluster Incl AF056194 Mus musculus short-chain dehydrogenase CRAD2 mRNA, complete cds/cds = (97,1047)/ | −70.8 | M | −220.5 | M | 22.4 | P |
| | | gb = AF056194/gi = 3294554/ug = Mm 6696/len = 1612/STRA = for | | | | | | |
| 16 | 103085_at | Cluster Incl AB013095 Mus musculus mRNA for heme-binding protein, complete cds/cds = (57,629)/gb = AB013095/ | 251.6 | P | 166.1 | A | 94.3 | A |
| | | gi = 3724327/ug = Mm 17249/len = 1033/STRA = for | | | | | | |
| 16 | 103804_at | Cluster Incl AB006960 Mus musculus mRECK mRNA, complete cds/cds = (6,2921)/gb = AB006960/gi = 3810870/ | 41.3 | P | 43.3 | A | 51.4 | A |
| | | ug = Mm 20862/len = 4094/STRA = for | | | | | | |
| 16 | 95568_at | Cluster Incl AI853412.UI-M-BH0-ajy-a-05-0-UI s1 Mus musculus cDNA, 3 end/clone = UI-M-BH0-ajy-a-05-0-UI/ | 19.3 | A | 23.9 | P | 25.8 | A |
| | | clone_end = 3/gb = AI853412/gi = 5497318/ug = Mm 27799/len = 587/STRA = for | | | | | | |
| 16 | 96810_at | Cluster Incl AI154017 ud58a11 r1 Mus musculus cDNA, 5 end/clone = IMAGE-1450076/clone_end = 5/gb = AI154017/ | 286.6 | P | 223.7 | A | 242.9 | A |
| | | gi = 3682486/ug = Mm 29266/len = 156/STRA = for | | | | | | |
| 16 | 104402_at | Cluster Incl AA030469 mi23c07 r1 Mus musculus cDNA, 5 end/clone = IMAGE-464364/clone_end = 5/gb = AA030469/ | 87.2 | A | 55.7 | A | 12.7 | A |
| | | gi = 1497632/ug = Mm 2869/len = 342/STRA = for | | | | | | |
| 16 | 93949_at | Cluster lncl M63658 M musculus G protein beta-subunit mRNA, complete cds/cds = (40,1062)/gb = M63658/gi = 193435/ | 48.2 | P | 9.6 | A | 38.3 | A |
| | | ug = Mm 9336/len = 1135/STRA = for | | | | | | |
| 16 | 98829_at | Cluster Incl X80417 Potassium inwardly rectifying channel, subfamily J, member 12/cds = (1,1284)/gb = X80417/gi = 550388/ | 106.4 | P | 22.6 | A | 7.3 | A |
| | | ug = Mm 4970/len = 1618/STRA = for | | | | | | |
| 16 | 102562_at | Cluster Incl Z80833 M musculus spermidine synthase pseudogene 2/cds = (0,852)/gb = Z80833/gi = 1592790/ug = Mm 57224/ | 498.3 | P | 357.8 | A | 434.7 | A |
| | | len = 853/STRA = for | | | | | | |
| 16 | 95513_at | Cluster Incl AW124310 UI-M-BH2.1-apq-d-03-0-UI s1 Mus musculus cDNA, 3 end/clone = UI-M-BH2 1 -apq-d-03-0-UI/ | 175.5 | A | 149.9 | M | 150.8 | P |
| | | clone_end = 3/gb = AW124310/gi = 6099840/ug = Mm.25298/len = 234/STRA = for | | | | | | |
| 16 | 95641_at | Cluster Incl AI853899 UI-M-BH0-aju-c-09-0-UI.s1 Mus musculus cDNA, 3 end/clone = UI-M-BH0-aju-c-09-0-UI/ | 313.2 | A | 88.8 | A | 195 | A |
| | | clone_end = 3/gb = AI853899/gi = 5497805/ug = Mm.29491/len = 380/STRA = for | | | | | | |
| 16 | 103043_at | Cluster Incl Z35294 M musculus MTCP-1 gene/cds = (540,746)/gb = Z35294/gi = 603859/ug = Mm 16366/len = 954/ | 132.3 | M | 24.7 | A | 27.8 | A |
| | | STRA = for | | | | | | |
| 16 | 103831_at | Cluster Incl U72194 Mus musculus muskelin mRNA, complete cds/cds = (12,2219)/gb = U72194/gi = 3493461/ | 116.1 | A | 52.6 | A | 127.2 | A |
| | | ug = Mm 20930/len = 3975/STRA = for | | | | | | |
| 16 | 103869_at | Cluster Incl U16175 Mus musculus thrombospondin 3 (Thbs3) gene, partial cds and mucin 1 (Muc1) gene, complete cds/ | 163.1 | P | 94.2 | A | 108.7 | A |
| | | cds = (0,371)/gb = U16175/gi = 608488/ug = Mm 2114/len = 615/STRA = for | | | | | | |
| 16 | 104042_at | Cluster Incl AI842275 UI-M-AM1-afz-d-12-0-UI s1 Mus musculus cDNA, 3 end/clone = UI-M-AM1-afz-d-12-0-UI/ | 237.5 | P | 82.6 | A | 238.9 | M |
| | | clone end = 3/gb = AI842275/gi = 5476528/ug = Mm 21470/len = 412/STRA = for | | | | | | |
| 16 | 104752_at | Cluster Incl AB028863 Mus musculus mRNA for MMRP19, complete cds/cds = (5,730)/gb = AB028863/gi = 5103282/ | 509.7 | A | 352.4 | A | 446.9 | A |
| | | ug = Mm.24772/len = 840/STRA = for | | | | | | |
| 16 | 94940_at | Cluster Incl AW123316 UI-M-BH2 1-apf-b-01-0-UI s1 Mus musculus cDNA, 3 end/clone = UI-M-BH2 1-apf-b-01-0-UI/ | 161.6 | A | 53.3 | A | 98.6 | A |
| | | clone_end = 3/gb = AW123316/gi = 6098846/ug = Mm.26925/len = 414/STRA = for | | | | | | |
| 16 | 100968_at | Cluster Incl AW122609.UI-M-BH2 2-aol-a-11-0-UI s1 Mus musculus cDNA, 3 end/clone = UI-M-BH2 2-aol-a-11-0-UI/ | 3.4 | A | −83.5 | A | 78.2 | A |
| | | clone_end = 3/gb = AW122609/gi = 6098136/ug = Mm.6626/len = 257/STRA = rev | | | | | | |
| 16 | 101939_at | Cluster Incl AI849344: UI-M-AJ1-aha-f-04-0-UI.s1 Mus musculus cDNA, 3 end/clone = UI-M-AJ1-aha-f-04-0-UI/ | 875.8 | P | 411.1 | A | 527.8 | P |
| | | clone_end = 3/gb = AI849344/gi = 5493250/ug = Mm.95187/len = 570/STRA = for | | | | | | |
| 16 | 101940_at | Cluster Incl AJ222800: Mus musculus mRNA for neutral sphingomyelinase/cds = (309,1568)/gb = AJ222800/gi = 3021479/ | 164.5 | A | 204.8 | A | 227.4 | A |
| | | ug = Mm 953/len = 1627/STRA = for | | | | | | |
| 16 | 103256_at | Cluster Incl AI509811: vx28b11 y1 Mus musculus cDNA, 5 end/clone = IMAGE-1265757/clone_end = 5/gb = AI509811/ | 240.7 | A | 156.8 | P | 114.7 | P |
| | | gi = 4408716/ug = Mm 2072/len = 251/STRA = for | | | | | | |
| 16 | 92314_at | Cluster Incl U25652: Procollagen, type XII, alpha 1/cds = (83,9286)/gb = U25652/gi = 953236/ug = Mm 3819/len = 9704/ | 78.8 | A | 108.8 | P | 83.2 | A |
| | | STRA = rev | | | | | | |
| 16 | 92482_at | Cluster Incl AF006466: Mus musculus lymphocyte specific formin related protein (Fr1) mRNA, complete cds/cds = (0,3194)/ | 152.2 | A | 194.5 | A | 187.5 | P |
| | | gb = AF006466/gi = 4101719/ug = Mm 42152/len = 3195/STRA = for | | | | | | |
| 16 | 96509_at | Cluster Incl J05019 Fc receptor, IgE, high affinity I, beta polypeptide/cds = (22,729)/gb = J05019/gi = 193238/ug = Mm 2544/ | 151.4 | A | 149 | A | 132.8 | A |
| | | len = 1010/STRA = for | | | | | | |
| 17 | AFFX-BioB-M_at | E Coli BioB middle | 402.9 | P | 487.3 | P | 619.9 | P |
| 17 | AFFX-BioC-5_at | E Coli BioC 5 prime | 911.7 | P | 1372.7 | P | 1190 | P |
| 17 | AFFX-BioDn-5_at | E Coli BioD 5 prime | 1165.7 | P | 1934.1 | P | 1562.3 | P |
| 17 | 92554_at | Cluster Incl AF059735 Mus musculus C-terminal binding protein 2 CtBP2 mRNA, complete cds/cds = (0,1337)/ | 373.9 | P | 502.4 | P | 494.6 | P |
| | | gb = AF059735/gi = 3513570/ug = Mm.10172/len = 1338/STRA = rev | | | | | | |
| 17 | 92593_at | Cluster Incl D13664 Mus musculus osf-2 mRNA for osteoblast specific factor 2, complete cds/cds = (18,2453)/gb = D13664/ | 2215.8 | P | 4038.7 | P | 3538.4 | P |
| | | gi = 393321/ug = Mm 10681/len = 3187/STRA = for | | | | | | |
| 17 | 92771_at | Cluster Incl AB013357 Mus musculus mRNA for 49 kDa zinc finger protein, complete cds/cds = (110,1492)/gb = AB013357/ | 1131.4 | P | 1631.2 | P | 1522.2 | P |
| | | gi = 4153885/ug = Mm 12236/len = 2082/STRA = for | | | | | | |
| 17 | 93067_f_at | Cluster Incl U62674 Mus musculus histone H2a 2-615 (H2a-615), and histone H3 2-615 (H3-615) genes, complete cds/ | 899.5 | P | 985.7 | P | 759.2 | P |
| | | cds = (43,435)/gb = U62674/gi = 1458142/ug = Mm.15686/len = 515/STRA = for | | | | | | |
| 17 | 93070_at | Cluster Incl AI847564 UI-M-AM1-afv-g-08-0-UI s1 Mus musculus cDNA, 3 end/clone = UI-M-AM1-afv-g-08-0-UI/ | 450.7 | P | 480.7 | P | 467.5 | P |
| | | clone_end = 3/gb = AI847564/gi = 5491470/ug = Mm 15701/len = 348/STRA = for | | | | | | |
| 17 | 93097_at | Cluster Incl U51805 Mus musculus liver arginase mRNA, complete cds/cds = (141,1112)/gb = U51805/gi = 1293092/ | 156.4 | P | 220.8 | P | 246.1 | P |
| | | ug = Mm 16423/len = 1221/STRA = for | | | | | | |
| 17 | 93266_at | Cluster Incl U04541 Mus musculus alpha-tropomyosin slow mRNA, complete cds/cds = (53,910)/gb = U04541/gi = 439845/ | 886.5 | P | 1287.3 | P | 1080.8 | P |
| | | ug = Mm 17306/len = 1091/STRA = for | | | | | | |
| 17 | 93325_at | Cluster Incl AI845735 UI-M-AQ1-aeb-b-07-0-UI s1 Mus musculus cDNA, 3 end/clone = UI-M-AQ1-aeb-b-07-0-UI/ | 283 | P | 233.9 | P | 276.4 | P |
| | | clone_end = 3/gb = AI845735/gi = 5489641/ug = Mm 18579/len = 362/STRA = for | | | | | | |
| 17 | 93588_at | Cluster Incl Z54179 M musculus mRNA (evolutionary conserved transcript)/cds = (221,802)/gb = Z54179/gi = 1655636/ | 114.9 | P | 183.2 | P | 108.4 | P |
| | | ug = Mm 2080/len = 1217/STRA = for | | | | | | |
| 17 | 93752_at | Cluster lncl AI848393: UI-M-AH1-ago-g-10-0-UI s1 Mus musculus cDNA, 3 end/clone = UI-M-AH1-ago-g-10-0-UI/ | 506.7 | P | 619.8 | P | 478.3 | P |
| | | clone_end = 3/gb = AI848393/gi = 5492294/ug = Mm 21118/len = 468/STRA = for | | | | | | |
| 17 | 93768_f_at | Cluster Incl AW124052: UI-M-BH2 1-apn-e-06-0-UI s1 Mus musculus cDNA, 3 end/clone = UI-M-BH2 1-apn-e-06-0-UI/ | 771.2 | P | 830.1 | P | 767.5 | P |
| | | clone_end = 3/gb = AW124052/gi = 6099582/ug = Mm 21183/len = 246/STRA = for | | | | | | |
| 17 | 93801 _at | Cluster Incl AA763275 vv89d02 r1 Mus musculus cDNA, 5 end/clone = IMAGE-1229571/clone_end = 5/gb = AA763275/ | 1528.8 | P | 4339.5 | P | 3450 | P |
| | | gi = 2813022/ug = Mm 21408/len = 357/STRA = for | | | | | | |
| 17 | 94044_at | Cluster Incl AA692708 vr54d07 s1 Mus musculus cDNA, 5 end/clone = IMAGE-1124461/clone_end = 5/gb = AA692708/ | 247.1 | P | 486.5 | P | 387.5 | P |
| | | gi = 2693646/ug = Mm 21964/len = 170/STRA = for | | | | | | |
| 17 | 94232_at | Cluster Incl AI849928: UI-M-BG0-ahz-e-10-0-UI s1 Mus musculus cDNA, 3 end/clone = UI-M-BG0-ahz-e-10-0-UI/ | 969.5 | P | 1459.4 | P | 1107.7 | P |
| | | clone_end = 3/gb = AI849928/gi = 5493834/ug = Mm 22288/len = 482/STRA = for | | | | | | |
| 17 | 94295_at | Cluster Incl AF017085 Mus musculus BAP-135 homolog (Diws1t) mRNA, complete cds/cds = (87,3023)/gb = AF017085/ | 1077 | P | 1061.2 | P | 1080.2 | P |
| | | gi = 2653820/ug = Mm 22593/len = 4091/STRA = for | | | | | | |
| 17 | 94296_s_at | Cluster Incl AF043220 Mus musculus TFII-I protein short form mRNA, alternatively spliced, complete cds/cds = (4,2880)/ | 200 | P | 254 | P | 162.1 | P |
| | | gb = AF043220/gi = 2895589/ug = Mm 22593/len = 2936/STRA = for | | | | | | |
| 17 | 96041_at | Cluster Incl AB016424 Mus musculus rbm3 mRNA, complete cds/cds = (85,546)/gb = AB016424/gi = 3810891/ug = Mm 2591/ | 4796.8 | P | 6132 | P | 5960.8 | P |
| | | len = 1158/STRA = for | | | | | | |
| 17 | 96104_at | Cluster Incl AI047107 uh58b11 r1 Mus musculus cDNA, 5 end/clone = IMAGE-1749597/clone_end = 5/gb = AI047107/ | 215.1 | P | 459.6 | P | 386.7 | P |
| | | gi = 3295394/ug = Mm 26879/len = 403/STRA = for | | | | | | |
| 17 | 96649_at | Cluster Incl AW046552 UI-M-BH1-alb-b-06-0-UI.s1 Mus musculus cDNA, 3 end/clone = UI-M-BH1-alb-b-06-0-UI/ | 291.2 | P | 352.5 | P | 334.9 | P |
| | | clone_end = 3/gb = AW046552/gi = 5907081/ug = Mm 27328/len = 517/STRA = for | | | | | | |
| 17 | 97857_at | Cluster Incl AW047762 UI-M-BH1-alk-d-04-0-UI s1 Mus musculus cDNA, 3 end/clone = UI-M-BH1-alk-d-04-0-UI/ | 239.7 | P | 304.2 | P | 272.3 | P |
| | | clone_end = 3/gb = AW047762/gi = 5908291/ug = Mm 28300/len = 387/STRA = for | | | | | | |
| 17 | 97888_at | Cluster Incl AI849676 UI-M-AL1-ahl-e-06-0-UI.s1 Mus musculus cDNA, 3 end/clone = UI-M-AL1-ahl-e-06-0-UI/ | 189.7 | P | 434.9 | P | 293.8 | P |
| | | clone_end = 3/gb = AI849676/gi = 5493582/ug = Mm.28397/len = 367/STRA = for | | | | | | |
| 17 | 97893_at | Cluster Incl AB017697 Mus musculus TLP21 mRNA for 21-kDa TBP-like protein, complete cds/cds = (272,832)/ | 106.2 | P | 115.4 | P | 90.4 | P |
| | | gi = AB017697/gi = 4218069/ug = Mm 28415/len = 1273/STRA = for | | | | | | |
| 17 | 97908_at | Cluster Incl AW120676 UI-M-BH2 3-anz-b-10-0-UI.s1 Mus musculus cDNA, 3 end/clone = UI-M-BH2.3-anz-b-10-0-UI/ | 1045.4 | P | 1456.9 | P | 1182.9 | P |
| | | clone_end = 3/gb = AW120676/gi = 6095974/ug = Mm.26474/len = 452/STRA = for | | | | | | |
| 17 | 98924_at | Cluster Incl Y08027 M musculus ART3 gene/cds = (0,1112)/gb = Y08027/gi = 2677615/ug = Mm 28637/len = 1113/STRA = for | 100.4 | P | 155 | P | 167.5 | P |
| 17 | 94537_at | Cluster Incl AI838859 UI-M-AN0-ack-c-06-0-UI s1 Mus musculus cDNA, 3 end/clone = UI-M-AN0-ack-c-06-0-UI/ | 1801.9 | P | 2159.6 | P | 1182.7 | P |
| | | clone_end = 3/gb = AI838859/gi = 5473072/ug = Mm 29057/len = 349/STRA = for | | | | | | |
| 17 | 94542_at | Cluster Incl AA929348: vz41b07.r1 Mus musculus cDNA, 5 end/clone = IMAGE-1329013/clone_end = 5/gb = AA929348/ | 209.2 | P | 266.8 | P | 183.2 | P |
| | | gi = 3078657/ug = Mm 29087/len = 166/STRA = for | | | | | | |
| 17 | 95056_r_at | Cluster Incl AW122747 UI-M-BH2.2-aot-g-01-0-UI s1 Mus musculus cDNA, 3 end/clone = UI-M-BH2 2-aot-g-01-0-UI/ | 109.4 | P | 228.7 | P | 158 | P |
| | | clone_end = 3/gb = AW122747/gi = 6098277/ug = Mm 29150/len = 468/STRA = for | | | | | | |
| 17 | 95078_at | Cluster Incl AI841400 UI-M-AH0-acx-a-06-0-UI.s1 Mus musculus cDNA, 3 end/clone = UI-M-AH0-acx-a-06-0-UI/ | 123.3 | P | 217.1 | P | 206.2 | P |
| | | clone_end = 3/gb = AI841400/gi = 5475613/ug = Mm 29213/len = 213/STRA = for | | | | | | |
| 17 | 95092_at | Cluster Incl J05479: Mouse calcineurin catalytic subunit mRNA, complete cds/cds = (75,1640)/gb = J05479/gi = 192347/ | 495.2 | P | 711.7 | P | 633.6 | P |
| | | ug = Mm.293/len = 2194/STRA = for | | | | | | |
| 17 | 95142_s_at | Cluster lncl U10407 Capping protein beta 1/cds = (18,836)/gb = U10407/gi = 500748/ug = Mm 2945/len = 1014/STRA = for | 757.7 | P | 1063.6 | P | 815.2 | P |
| 17 | 95646_at | Cluster Incl U01170 Camiline palmitoyltransferase 2/cds = (0,1976)/gb = U01170/gi = 425266/ug = Mm 29499/len = 1977/ | 688.4 | P | 798.4 | P | 740 | P |
| | | STRA = for | | | | | | |
| 17 | 95730_at | Cluster Incl AI840884 UI-M-AM0-adm-b-06-0-UI s1 Mus musculus cDNA, 3 end/clone = UI-M-AM0-adm-b-06-0-UI/ | 791.6 | P | 853.2 | P | 863.7 | P |
| | | clone_end = 3/gb = AI840884/gi = 5475097/ug = Mm 29741/len = 317/STRA = for | | | | | | |
| 17 | 96299_at | Cluster Incl AIB47298: UI-M-AN1-afh-c-08-0-UI s1 Mus musculus cDNA, 3 end/clone = UI-M-AN1-afh-c-08-0-UI/ | 1475.9 | P | 2261.6 | P | 1997.3 | P |
| | | clone_end = 3/gb = AI847298/gi = 5491204/ug = Mm 29909/len = 328/STRA = for | | | | | | |
| 17 | 96322_at | Cluster Incl AI836001 UI-M-AJ0-abb-b-07-0-UI s1 Mus musculus cDNA, 3 end/clone = UI-M-AJ0-abb-b-07-0-UI/ | 731.5 | P | 764.1 | P | 642.8 | P |
| | | clone_end = 3/gb = AI836001/gi = 5470254/ug = Mm 29945/len = 481/STRA = for | | | | | | |
| 17 | 97518_at | Cluster Incl D29016 Mouse mRNA for squalene synthase/cds = (20,1270)/gb = D29016/gi = 468456/ug = Mm 3204/len = 1634/ | 296.1 | P | 775.5 | P | 480.6 | P |
| | | STRA/ = for | | | | | | |
| 17 | 98039_at | Cluster Incl AI853819 UI-M-BH0-ajr-a-08-0-UI s1 Mus musculus cDNA, 3 end/clone = UI-M-BH0-ajr-a-08-0-UI/ | 521 | P | 613.6 | P | 579.8 | P |
| | | clone_end = 3/gb = AI853819/gi = 5497725/ug = Mm 34002/len = 369/STRA = for | | | | | | |
| 17 | 98138_at | Cluster Incl AI854776 UI-M-BH0-aka-d-05-0-UI s1 Mus musculus cDNA, 3 end/clone = UI-M-BH0-aka-d-05-0-UI/ | 288.6 | P | 332.5 | P | 357.4 | P |
| | | clone_end = 3/gb = AI854776/gi = 5498682/ug = Mm.35538/len = 333/STRA = for | | | | | | |
| 17 | 98604_at | Cluster Incl U79774 Mus musculus NNP-1 var (NNP-1) mRNA, complete cds/cds = (43,1527)/gb = U79774/gi = 2258277/ | 424.4 | P | 489.4 | P | 432.6 | P |
| | | ug = Mm 38344/len = 1946/STRA = for | | | | | | |
| 17 | 99102_at | Cluster Incl U67874.Ubiquitin specific protease 9, X chromosome/cds = (355,8034)/gb = U67874/gi = 1527198/ug = Mm 396/ | 54.9 | P | 105.4 | P | 52.3 | P |
| | | len = 10323/STRA = for | | | | | | |
| 17 | 99121_at | Cluster Incl X90875 M musculus mRNA for FXR1 protein/cds = (13,1632)/gb = X90875/gi = 1122421/ug = Mm 4038/ | 192.9 | P | 180.1 | P | 139.7 | P |
| | | len = 2060/STRA = for | | | | | | |
| 17 | 99148_at | Cluster Incl AI852862 UI-M-BH0-ajk-e-02-0-UI s1 Mus musculus cDNA, 3 end/clone = UI-M-BH0-ajk-e-02-0-UI/ | 500.1 | P | 928.2 | P | 1293.6 | P |
| | | clone_end = 3/gb = AI852862/gi = 5496768/ug = Mm.41502/len = 266/STRA = for | | | | | | |
| 17 | 96104_at | Cluster Incl AI047107 uh58b11 r1 Mus musculus cDNA, 5 end/clone = IMAGE-1749597/clone_end = 5/gb = AI047107/ | 215.1 | P | 459.6 | P | 386.7 | P |
| 17 | 99184_at | Cluster Incl AW120896 UI-M-BH2.3-aob-b-10-0-UI s1 Mus musculus cDNA, 3 end/clone = UI-M-BH2 3-aob-b-10-0-UI/ | 473.2 | P | 556.8 | P | 732.6 | P |
| | | clone_end = 3/gb = AW120896/gi = 6096229/ug = Mm 41853/len = 302/STRA = for | | | | | | |
| 17 | 99580_s_at | Cluster Incl U16818 Mus musculus UDP glucuronosyltransferase (UGT1-06) mRNA, complete cds/cds = (128,1723)/ | 1008 | P | 1177.4 | P | 1380.6 | P |
| | | gb = U16818/gi = 801898/ug = Mm 42472/len = 2300/STRA = for | | | | | | |
| 17 | 99582_at | Cluster Incl M76124 Mus musculus EGP314 precursor mRNA, complete cds/cds = (107,1051)/gb = M76124/gi = 193007/ | 91.1 | P | 154.7 | P | 103.8 | P |
| | | ug = Mm 4259/len = 1710/STRA = for | | | | | | |
| 17 | 99666_at | Cluster Incl AW125431: UI-M-BH2.3-aqh-g-01-0-UI s1 Mus musculus cDNA, 3 end/clone = UI-M-BH2 3-aqh-g-01-0-UI/ | 2204.5 | P | 2406.9 | P | 2215.4 | P |
| | | clone_end = 3/gb = AW125431/gi = 6100961/ug = Mm 43822/len = 406/STRA = for | | | | | | |
| 17 | 100587_f_at | Cluster Incl AI843959 UI-M-AP1-agh-e-01-0-UI s1 Mus musculus cDNA, 3 end/clone = UI-M-AP1-agh-e-01-0-UI/ | 216.9 | P | 249.6 | P | 231.8 | P |
| | | clone_end = 3/gb = AI843959/gi = 5478172/ug = Mm 622/len = 379/STRA = for | | | | | | |
| 17 | 101557_at | Cluster Incl AF043070 Mus musculus branched chain alpha ketoacid dehydrogenase kinase (bckd-kinase) mRNA, nuclear | 1085.6 | P | 1388.6 | P | 1119.7 | P |
| | | gene encoding mitochondrial protein, complete cds/cds = (106,1344)/gb = AF043070/gi = 2809219/ug = Mm.8903/len = 1654/ | | | | | | |
| | | STRA = for | | | | | | |
| 17 | 101564_at | Clusler Incl U21855 Mus musculus mCAF1 protein mRNA, complete cds/cds = (39,896)/gb = U21855/gi = 726135/ | 127 | P | 126.3 | P | 113.1 | P |
| | | ug = Mm 89351/len = 1264/STRA = for | | | | | | |
| 17 | 101962_at | Cluser Incl AI854884: UI-M-BH0-akb-f-11-0-UI s1 Mus musculus cDNA, 3 end/clone = UI-M-BH0-akb-f-11-0-UI/ | 540.9 | P | 1467.6 | P | 867.2 | P |
| | | clone_end = 3/gb = AI854884/gi = 5498790/ug = Mm 92820/len = 407/STRA = for | | | | | | |
| 17 | 101990_at | Cluster Incl X51905: Lactate dehydrogenase 2, B chain/cds = (46,1050)/gb = X51905/gi = 52879/ug = Mm 9745/len = 1238/ | 300 | P | 272.4 | P | 377.5 | P |
| | | STRA = for | | | | | | |
| 17 | 102018_at | Cluster Incl AI854879 UI-M-BH0-akb-f-05-0-UI.s1 Mus musculus cDNA, 3 end/clone = UI-M-BH0-akb-f-05-0-UI/ | 304.2 | P | 377.1 | P | 342.3 | P |
| | | clone_end = 3/gb = AI854879/gi = 5498785/ug = Mm 10038/len = 127/STRA = for | | | | | | |
| 17 | 102054_at | Cluster Incl AB011370.Mus musculus Ankhzn mRNA, complete cds/cds = (21,3575)/gb = AB011370/gi = 2914016/ | 223.1 | P | 356.7 | P | 326.6 | P |
| | | ug = Mm 10313/len = 7143/STRA = for | | | | | | |
| 17 | 102060_at | Cluster Incl AF051357 Mus musculus golgin-245 (olp-1) mRNA, complete cds/cds = (87,3743)/gb = AF051357/gi = 2952521/ | 337.5 | P | 577.3 | P | 367.5 | P |
| | | ug = Mm 10409/len = 4393/STRA = rev | | | | | | |
| 17 | 102812_I_at | Cluster Incl AW210346 uI53g12 y1 Mus musculus cDNA, 5 end/clone = IMAGE-2102086/clone_end = 5/gb = AW210346/ | 355.4 | P | 427.5 | P | 361.1 | P |
| | | gi = 6516286/ug = Mm.14253/len = 431/STRA = for | | | | | | |
| 17 | 103352_at | Cluster Incl X65603 Dolichyl-phosphate alpha-N-acetylglucosaminephosphotransferase 2/cds = (59,1291)/gb = X65603/ | 135.9 | P | 387.6 | P | 250.4 | P |
| | | gi = 51077/ug = Mm 18353/len = 1764/STRA = for | | | | | | |
| 17 | 103421_at | Cluster Incl D50464.Mouse SDR2 mRNA, complete cds/cds = (128,1906)/gb = D50464/gi = 1747305/ug = Mm.18910/ | 101.9 | P | 266.9 | P | 162.9 | P |
| | | len = 2394/STRA = for | | | | | | |
| 17 | 103424_at | Cluster Incl AI844839 UI-M-BG0-ahv-a-06-0-UI s1 Mus musculus cDNA, 3 end/clone = UI-M-BG0-ahv-a-06-0-UI/ | 304.1 | P | 441.1 | P | 478 | P |
| | | clone_end = 3/gb = AI844839/gi = 5488785/ug = Mm 18957/len = 423/STRA = for | | | | | | |
| 17 | 103429_i_at | Cluster Incl AW125330 UI-M-BH2 1-apy-e-12-0-UI s1 Mus musculus cDNA, 3 end/clone = UI-M-BH2 1-apy-e-12-0-UI/ | 77.8 | P | 143.3 | P | 60.6 | P |
| | | clone_end = 3/gb = AW125330/gi = 6100855/ug = Mm 19006/len = 208/STRA = for | | | | | | |
| 17 | 103567_at | Cluster Incl AW123810 UI-M-BH2 1-apm-f-05-0-UI s1 Mus musculus cDNA, 3 end/clone = UI-M-BH2.1-apm-f-05-0-UI/ | 462.4 | P | 701.2 | P | 597 | P |
| | | clone_end = 3/gb = AW123810/gi = 6099340/ug = Mm 1921/len = 404/STRA = for | | | | | | |
| 17 | 103585_at | Cluster Incl AI851408 UI-M-BH0-akl-b-03-0-UI s1 Mus musculus cDNA, 3 end/clone = UI-M-BH0-alq-b-03-0-UI/ | 154 | P | 205.2 | P | 152.2 | P |
| | | clone_end = 3/gb = AI851408/gi = 5495314/ug = Mm 19804/len = 342/STRA = for | | | | | | |
| 17 | 103648_at | Cluster Incl Y08830 M musculus trop2 gene/cds = (0,953)/gb = Y08830/gi = 2764995/ug = Mm.20410/len = 954/STRA = for | 1519.9 | P | 1699.9 | P | 1222.1 | P |
| 17 | 103672_at | Cluster Incl AW122572 UI-M-BH2 2-aox-f-06-0-UI s1 Mus musculus cDNA, 3 end/clone = UI-M-BH2 2-aox-f-06-0-UI/ | 287.3 | P | 414.7 | P | 481.8 | P |
| | | clone_end = 3/gb = AW122572/gi = 6098097/ug = Mm20805/len = 505/STRA = for | | | | | | |
| 17 | 103845_at | Cluster Incl AI839005 UI-M-AN0-acm-c-06-0-UI s1 Mus musculus cDNA, 3 end/clone = UI-M-AN0-acm-c-06-0-UI/ | 258.2 | P | 480.7 | P | 438.1 | P |
| | | clone_end = 3/gb = AI839005/gi = 5473218/ug = Mm 2097/len = 447/STRA = rev | | | | | | |
| 17 | 103912_at | Cluster Incl AW046563: UI-M-BH1-alb-c-08-0-UI.s1 Mus musculus cDNA, 3 end/clone = UI-M-BH1-alb-c-08-0-UI/ | 426.3 | P | 472.4 | P | 496.7 | P |
| | | clone_end = 3/gb = AW046563/gi = 5907092/ug = Mm.21428/len = 520/STRA = for | | | | | | |
| 17 | 104050_at | Cluster Incl AI846060: UI-M-AK1-aew-g-02-0-UI s1 Mus musculus cDNA, 3 end/clone = UI-M-AK1-aew-g-02-0-UI/ | 695.3 | P | 973.8 | P | 787.4 | P |
| | | clone_end = 3/gb = AI846060/gi = 5490006/ug = Mm 21515/len = 229/STRA = rev | | | | | | |
| 17 | 104125_at | Cluster Incl AA763673 vp09g09 r1 Mus musculus cDNA, 5 end/clone = IMAGE-1068160/clone_end = 5/gb = AA763673/ | 483 | P | 599.9 | P | 812 | P |
| | | gi = 2813755/ug = Mm 22030/len = 568/STRA = for | | | | | | |
| 17 | 104337_f_at | Cluster Incl AW208938 uo65g07 x1 Mus musculus cDNA, 3 end/clone = IMAGE-2647452/clone_end = 3/gb = AW208938/ | 268.9 | P | 336.8 | P | 278.7 | P |
| | | gi = 6514878/ug = Mm 2252/len = 715/STRA = for | | | | | | |
| 17 | 104340_at | Cluster Incl AF072240 Mus musculus methyl-CpG binding protein MBD1(Mbd1) mRNA, complete cds/cds = (175,2085)/ | 114.4 | P | 135.8 | P | 113.3 | P |
| | | gb = AF072240/gi = 3800788/ug = Mm 22522/len = 2820/STRA = for | | | | | | |
| 17 | 104522_at | Cluster Incl AI047617: Uh63h03.r1 Mus musculus cDNA, 5 end/clone = IMAGE-1750133/clone_end = 5/gb = AI047617/ | 181.8 | P | 254.6 | P | 302.5 | P |
| | | gi = 3295904/ug = Mm 22930/len = 344/STRA = for | | | | | | |
| 17 | 104557_at | Cluster Incl AI847097 UI-M-AP1-agn-f-01-0-UI s1 Mus musculus cDNA, 3 end/clone = UI-M-AP1-agn-f-01-0-UI/ | 607.2 | P | 723.3 | P | 490.9 | P |
| | | clone_end = 3/gb = AI847097/gi = 5491003/ug = Mm 23807/len = 203/STRA = rev | | | | | | |
| 17 | 104577_at | Cluster Incl AA920621 vz07106 r1 Mus musculus cDNA, 5 end/clone = IMAGE-1315043/clone_end = 5/gb = AA920621/ | 70.5 | P | 142.4 | P | 87.1 | P |
| | | gi = 3067400/ug = Mm 23949/len = 294/STRA = for | | | | | | |
| 17 | 104582_g_at | Cluster Incl AI845438 UI-M-AO1-aej-d-10-0-UI s1 Mus musculus cDNA, 3 end/clone = UI-M-AO1-aej-d-10-0-UI/ | 121.8 | P | 148.5 | P | 153.5 | P |
| | | clone_end = 3/gb = AI845438/gi = 5489339/ug = Mm 23975/len = 369/STRA = for | | | | | | |
| 17 | 104589_at | Cluster Incl AF091096 Mus musculus NNX3 mRNA, complete cds/cds = (76,1449)/gb = AF091096/gi = 4165096/ | 765 | P | 648.5 | P | 657.8 | P |
| | | ug = Mm 23997/len = 2044/STRA = for | | | | | | |
| 17 | 104604_at | Cluster Incl U62908 Mus musculus zinc linger protein 96 (Zfp96) mRNA, complete cds/cds = (432,1937)/gb = U62908/ | 79.3 | P | 92.2 | P | 95.6 | P |
| | | gi = 4097500/ug = Mm.24124/len = 2307/STRA = for | | | | | | |
| 17 | 104611_at | Cluster Incl AI854008 UI-M-BH0-arv-h-12-0-UI s1 Mus musculus cDNA, 3 end/clone = UI-M-BH0-arv-h-12-0-UI/ | 289.1 | P | 402.6 | P | 342.3 | P |
| | | clone_end = 3/gb = AI854008/gi = 5497914/ug = Mm 24158/len = 494/STRA = for | | | | | | |
| 17 | 94352_at | Cluster Incl AI850675 UI-M-BG1-ail-a-01-0-UI s1 Mus musculus cDNA, 3 end/clone = UI-M-BG1-ail-a-01-0-UI/ | 278.8 | P | 554.5 | P | 282.3 | P |
| | | clone_end = 3/gb = AI850675/gi = 5494581/ug = Mm 25200/len = 215/STRA = for | | | | | | |
| 17 | 94426_at | Cluster Incl AI851052 UI-M-BH0-ajv-g-02-0-UI s1 Mus musculus cDNA, 3 end/clone = UI-M-BH0-ajv-g-02-0-UI/ | 626.7 | P | 734.1 | P | 619.1 | P |
| | | clone_end = 3/gb = AI851052/gi = 5494958/ug = Mm.26392/len = 404/STRA = for | | | | | | |
| 17 | 95528_at | Cluster Incl AA791234 vv92e10 r1 Mus musculus cDNA, 5 end/clone = IMAGE-1229898/clone_end = 5/gb = AA791234/ | 541.6 | P | 958.2 | P | 622.5 | P |
| | | gi = 2851354/ug = Mm 27613/len = 347/STRA = for | | | | | | |
| 17 | 95571_at | Cluster Incl AF004100: Zinc transporter 4/cds = (0,600)/gb = AF004100/gi = 2580599/ug = Mm 27801/len = 620/STRA = rev | −141.4 | P | 184.1 | P | 166.2 | P |
| 17 | 95577_at | Custer Incl AW049625 UI-M-BH1-and-e-07-0-UI s1 Mus musculus cDNA, 3 end/clone = UI-M-BH1-and-e-07-0-UI/ | 271.9 | P | 527.3 | P | 597 | P |
| | | clone_end = 3/gb = AW049625/gi = 5910154/ug = Mm 27829/len = 352/STRA = rev | | | | | | |
| 17 | 95616_at | Cluster Incl AA674714 vm74b12 s1 Mus musculus cDNA, 5 end/clone = IMAGE-1003967/clone_end = 5/gb = AA674714/ | 111 | P | 117.1 | P | 91.5 | P |
| | | gi = 2651951/ug = Mm 28020/len = 156/STRA = for | | | | | | |
| 17 | 95637_at | Cluster Incl AI838592.UI-M-AO0-abz-e-02-0-UI s1 Mus musculus cDNA, 3 end/clone = UI-M-AO0-abz-e-02-0-UI/ | 379.6 | P | 484.2 | P | 358.4 | P |
| | | clone_end = 3/gb = AI838592/gi = 5472805/ug = Mm 28095/len = 321/STRA = for | | | | | | |
| 17 | 96144_at | Cluster Incl AJ001972 Mus musculus Id4 dominant negative helix-loop-helix gene/cds = (0,485)/gb = AJ001972/gi = 2546999/ | 367.5 | P | 545.7 | P | 219.7 | P |
| | | ug = Mm 28223/len = 486/STRA = for | | | | | | |
| 17 | 96151_at | Cluster Incl AA839813 vw52c02 r1 Mus musculus cDNA, 5 end/clone = IMAGE-1247426/clone_end = 5/gb = AA839813/ | 276 | P | 329.7 | P | 289.1 | P |
| | | gi = 2916407/ug = Mm 28252/len = 527/STRA = for | | | | | | |
| 17 | 96166_at | Cluster Incl AI842524 UI-M-AQ1-aea-c-06-0-UI s1 Mus musculus cDNA, 3 end/clone = UI-M-AQ1-aea-c-06-0-UI/ | 571.3 | P | 676 | P | 499.7 | P |
| | | clone_end = 3/gb = AI842524/gi = 5476737/ug = Mm 28371/len = 440/STRA = for | | | | | | |
| 17 | 96217_at | Cluster Incl AW060552: UI-M-BH1-ann-g-10-0-UI.s1 Mus musculus cDNA, 3 end/clone = UI-M-BH1-ann-g-10-0-UI/ | 176.2 | P | 325.8 | P | 185.9 | P |
| | | clone_end = 3/gb = AW060552/gi = 6008303/ug = Mm 28684/len = 334/STRA = for | | | | | | |
| 17 | 96724_r_at | Cluster Incl AW122911 UI-M-BH2 1-apa-f-09-0-UI.s1 Mus musculus cDNA, 3 end/clone = UI-M-BH2 1-apa-f-09-0-UI/ | 423.3 | P | 485.5 | P | 409.4 | P |
| | | clone_end = 3/gb = AW122911/gi = 6098441/ug = Mm 28829/len = 366/STRA = for | | | | | | |
| 17 | 96734_at | Cluster Incl AA674143 vp96d10 r1 Mus musculus cDNA, 5 end/clone = IMAGE-1092595/clone_end = 5/gb = AA674143/ | 461 | P | 574.9 | P | 441.7 | P |
| | | gi-2651380/ug = Mm.28894/len = 474/STRA = for | | | | | | |
| 17 | 96736_at | Cluster Incl AI850370 UI-M-BG1-aig-c-02-0-UI s1 Mus musculus cDNA, 3 end/clone = UI-M-BG1-aig-c-02-0-UI/ | 852.9 | P | 1248.7 | P | 812.5 | P |
| | | clone_end = 3/gb = AI850370/gi = 5494276/ug = Mm 28900/len = 432/STRA = rev | | | | | | |
| 17 | 96750_at | Cluster Incl AW122179 UI-M-BH2 2-aov-a-01-0-UI s1 Mus musculus cDNA, 3 end/clone = UI-M-BH2 2-aov-a-01-0-UI/ | 308.4 | P | 403.3 | P | 280.2 | P |
| | | clone_end = 3/gb = AW122179/gi = 6097626/ug = Mm 28965/len = 496/STRA = for | | | | | | |
| 17 | 96781_at | Cluster Incl AI838293.UI-M-AO0-aby-c-04-0-UI s1 Mus musculus cDNA, 3 end/clone = UI-M-AO0-aby-c-04-0-UI/ | 367.1 | P | 459.2 | P | 431.5 | P |
| | | clone_end = 3/gb = AI838293/gi = 5472506/ug = Mm 29072/len = 310/STRA = for | | | | | | |
| 17 | 98020_at | Cluster Incl D10715.Mouse mRNA for novel GTP-binding protein, complete cds/cds = (76,1179)/gb = D10715/gi = 220506/ | 944.6 | P | 828.4 | P | 871.8 | P |
| | | ug = Mm.3250/len = 1524/STRA = for | | | | | | |
| 17 | 98032_at | Cluster Incl M36146 Zinc finger protein 35/cds = (164,1906)/gb = M36146/gi = 202451/ug = Mm.3297/len = 2293/STRA = for | 188 | P | 202.5 | P | 176.4 | P |
| 17 | 98397_at | Cluster Incl AI852694 UI-M-BH0-aji-d-08-0-UI.s1 Mus musculus cDNA, 3 end/clone = UI-M-BH0-aji-d-08-0-UI/clone_end = 3/ | 217.2 | P | 339.2 | P | 194.1 | P |
| | | gb = AI852694/gi = 5496600/ug = Mm 33285/len = 495/STRA = for | | | | | | |
| 17 | 98464_at | Cluster Incl AW124196 UI-M-BH2 1-aph-b-03-0-UI s1 Mus musculus cDNA, 3 end/clone = UI-M-BH2 1-aph-b-03-0-UI/ | 185 | P | 255.4 | P | 261.2 | P |
| | | clone_end = 3/gb = AW124196/gi = 6099726/ug = Mm 34812/len 382/STRA = rev | | | | | | |
| 17 | 98486_at | Cluster Incl AF105222 Mus musculus slathmin-like-protein RB3 mRNA, complete cds/cds = (88,657)/gb = AF105222/ | 205 | P | 660.7 | P | 495.8 | P |
| | | gi = 4028597/ug = Mm 35474/len = 1197/STRA = rev | | | | | | |
| 17 | 98982_at | Cluster Incl AW046443 UI-M-BH1-akp-e-04-0-UI s1 Mus musculus cDNA, 3 end/clone = UI-M-BH1-akp-e-04-0-UI/ | 451.3 | P | 856.4 | P | 387.1 | P |
| | | clone_end = 3/gb = AW046443/gi = 5906972/ug = Mm.36912/len = 439/STRA = for | | | | | | |
| 17 | 99485_at | Cluster Incl AB009376 Mus musculus mRNA for ICAD-S, complete cds/cds = (0,797)/gb = AB009376/gi = 2754587/ | 223.9 | P | 331 | P | 284.6 | P |
| | | ug = Mm 41433/len = 798/STRA = for | | | | | | |
| 17 | 99536_at | Cluster Incl AB016080 Mus musculus mRNA for calcium binding protein Ktp 2, complete cds/cds = (61,624)/gb = AB016080/ | 145.4 | P | 205.4 | P | 165 | P |
| | | gi = 4115809/ug = Mm.42192/len = 736/STRA = for | | | | | | |
| 17 | 99549_at | Cluster Incl D31951: Mouse mRNA for osteoglycin, complete cds/cds = (39,935)/gb = D31951/gi = 971273/ug = Mm.4258/ | 146.9 | P | 127.8 | P | 95.7 | P |
| | | len = 1268/STRA = for | | | | | | |
| 17 | 99975_at | Cluster Incl AA879937: vw02b10.r1 Mus musculus cDNA, 5 end/clone = IMAGE-1230715/clone_end = 5/gb = AA879937/ | 296 | P | 452.8 | P | 484.3 | P |
| | | gi = 2988920/ug = Mm 4428/len = 331/STRA = for | | | | | | |
| 17 | 100009_r_at | Cluster Incl X94127 SRY-box containing gene 2/cds = (0,959)/gb = X94127/gi = 1209429/ug = Mm 4541/len = 960/STRA = for | 68.2 | P | 89.4 | P | 71.7 | P |
| 17 | 100496_at | Cluster Incl U79523 Mus musculus peptidylglycine alpha-amidating monooxygenase (PAM) mRNA, complete cds/ | 467.9 | P | 579.7 | P | 360.9 | P |
| | | cds = (335,3274)/gb = U79523/gi = 1711198/ug = Mm.5121/len = 4032/STRA = for | | | | | | |
| 17 | 101877_at | Cluster Incl AI854432 UI-M-BH0-ail-c-06-0-UI s1 Mus musculus cDNA, 3 end/clone = UI-M-BH0-ail-c-06-0-UI/clone_end = 3/ | 370.3 | P | 589.4 | P | 463.1 | P |
| | | gb = AI854432/gi = 5498338/ug = Mm 88182/len = 413/STRA = rev | | | | | | |
| 17 | 101912_at | Cluster Incl AI019679 ua92e01.r1 Mus musculus cDNA, 5 end/clone = IMAGE-1364952/clone_end = 5/gb = AI019679/ | 1218.6 | P | 1902.3 | P | 1634.5 | P |
| | | gi = 3234015/ug = Mm 902/len = 363/STRA = for | | | | | | |
| 17 | 102215_at | Cluster Incl Y10495 Mus musculus mRNA for CDV-1R protein/cds = (886,2130)/gb = Y10495/gi = 2995446/ug = Mm 10125/ | 194.6 | P | 230.3 | P | 160.7 | P |
| | | len = 2649/STRA = for | | | | | | |
| 17 | 102894_g_at | Cluster Incl X68363 POU domain, class 2, transcription factor 1/cds = (52,2292)/gb = X68363/gi = 53471/ug = Mm 15568/ | 163.9 | P | 289.4 | P | 261.2 | P |
| | | len = 2574/STRA = for | | | | | | |
| 17 | 102980_at | Cluster Incl AI849119 UI-M-AJ1-agy-a-03-0-UI s1 Mus musculus cDNA, 3 end/clone = UI-M-AJ1-agy-a-03-0-UI/ | 262.8 | P | 551.7 | P | 309.9 | P |
| | | clone_end = 3/gb = Al849119/gi = 5493025/ug = Mm.1896/len = 507/STRA = rev | | | | | | |
| 17 | 103285_at | Cluster Incl AF072249.Mus musculus methyl-CpG binding protein MBD4 (Mbd4) mRNA, complete cds/cds = (148,1812)/ | 92 | P | 122.2 | P | 80.7 | P |
| | | gb = AF072249/gi = 3800806/ug = Mm 20890/len = 1955/STRA = for | | | | | | |
| 17 | 103713_at | Cluster Incl AW124533 UI-M-BH2 1-apo-g-12-0-UI s1 Mus musculus cDNA, 3 end/clone = UI-M-BH2 1-apo-g-12-0-UI/ | 377.6 | P | 907.4 | P | 589 | P |
| | | clone_end = 3/gb = AW124533/gi = 6100063/ug = Mm 24116/len = 379/STRA = for | | | | | | |
| 17 | 103780_at | Cluster Incl AW049510 UI-M-BH1-anf-b-05-0-UI s1 Mus musculus cDNA, 3 end/clone = UI-M-BH1-anf-b-05-0-UI/ | 159.2 | P | 171.3 | P | 180.6 | P |
| | | clone_end = 3/gb = AW049510/gi = 5910039/ug = Mm 24863/len = 458/STRA = for | | | | | | |
| 17 | 103781_at | Cluster Incl U76832 Mus musculus plasma membrane protein syntaxin-4 mRNA, complete cds/cds = (0,896)/gb = U76832/ | 456.1 | P | 616.4 | P | 453.2 | P |
| | | gi = 1673624/ug = Mm 24867/len = 897/STRA = for | | | | | | |
| 17 | 103916_at | Cluster Incl AI850713 UI-M-BG1-ail-d-10-0-UI s1 Mus musculus cDNA, 3 end/clone = UI-M-BG1-ail-d-10-0-UI/ | 220.4 | P | 368.7 | P | 301.1 | P |
| | | clone_end = 3/gb = AI850713/gi = 5494619/ug = Mm 24984/len = 321/STRA = rev | | | | | | |
| 17 | 103981_at | Cluster Incl AI842281 UI-M-AM1-afz-e-07-0-UI s1 Mus musculus cDNA, 3 end/clone = UI-M-AM1-afz-e-07-0-UI/ | 169.4 | P | 315 | P | 132.5 | P |
| | | clone_end = 3/gb = AI842281/gi = 5476534/ug = Mm 25837/len = 457/STRA = for | | | | | | |
| 17 | 104652_at | Cluster Incl AI849601 UI-M-AH1-agr-f-05-0-UI s1 Mus musculus cDNA, 3 end/clone = UI-M-AH1-agr-f-05-0-UI/ | 151.1 | P | 167.3 | P | 128.6 | P |
| | | clone_end = 3/gb = AI849601/gi = 5493507/ug = Mm 31570/len = 357/STRA = rev | | | | | | |
| 17 | 92241_at | Cluster Incl AI849075.UI-M-AH1-agw-e-03-0-UI s1 Mus musculus cDNA, 3 end/clone = UI-M-AH1-agw-e-03-0-UI/ | 592.8 | P | 668.1 | P | 571.5 | P |
| | | clone_end = 3/gb = AI849075/gi = 5492981/ug = Mm 34844/len = 355/STRA = rev | | | | | | |
| 17 | 92350_at | Cluster Incl AA165759 ms60e10 r1 Mus musculus cDNA, 5 end/clone = IMAGE-615978/clone_end = 5/gb = AA165759/ | 502.7 | P | 667.6 | P | 692.6 | P |
| | | gi = 1743974/ug = Mm 3926/len = 346/STRA = rev | | | | | | |
| 17 | 92495_at | Cluster Incl U71269 Mus musculus potential transcriptional repressor No14hp (Not4h) mRNA, complete cds/ | 211 | P | 333.8 | P | 185.6 | P |
| | | cds = (314,2041)/gb = U71269/gi = 4097901/ug = Mm 42183/len = 2319/STRA = rev | | | | | | |
| 17 | 92698_at | Cluster Incl U21301 Mus musculus c-mer tyrosine kinase receptor mRNA, complete cds/cds = (49,3033)/gb = U21301/ | 124.7 | P | 180.4 | P | 147.6 | P |
| | | gi = 885969/ug = Mm 4582/len = 3564/STRA = for | | | | | | |
| 17 | 92904_at | Cluster Incl U08185 B lymphocyte induced maturation protein/cds = (220,2790)/gb = U08185/gi = 474283/ug = Mm 4800/ | 214.2 | P | 278.2 | P | 198.6 | P |
| | | len = 5281/STRA = rev | | | | | | |
| 17 | 92986_g_at | Cluster Incl D83203 Mus musculus mRNA, one isoform of PTP-RL9/cds = UNKN0WN/gb = D83203/gi = 4092839/ | 341.3 | P | 535.2 | P | 448.4 | P |
| | | ug = Mm 5046/len = 4208/STRA = for | | | | | | |
| 17 | 93169_at | Cluster Incl AW212708 uo10g07.x1 Mus musculus cDNA, 3 end/clone = IMAGE-2599164/clone_end = 3/gb = AW212708/ | 277 | P | 462.2 | P | 300.8 | P |
| | | gi = 6518827/ug = Mm 5343/len = 465/STRA = for | | | | | | |
| 17 | 93456_r_at | Cluster Incl L47480.Mus musculus BMP-4 gene, complele cds/cds = (92,1354)/gb = L47480/gi = 994733/ug = Mm 6813/ | 254.1 | P | 530.4 | P | 230.6 | P |
| | | len = 1654/STRA = for | | | | | | |
| 17 | 93623_at | Cluster Incl U56724 Mus musculus dystrophin gene, exon 23 and partial cds/cds = (0,212)/gb = U56724/gi = 1336044/ | 103.8 | P | 175.7 | P | 112.7 | P |
| | | ug = Mm 742/len = 213/STRA = for | | | | | | |
| 17 | 93962_at | Cluster Incl AI848598 UI-M-AH1-agv-b-04-0-UI s1 Mus musculus cDNA, 3 end/clone = UI-M-AH1-agv-b-04-0-UI/ | 134.9 | P | 160.9 | P | 170 | P |
| | | clone_end = 3/gb = AI848598/gi = 5492544/ug = Mm 967/len = 470/STRA = for | | | | | | |
| 17 | 95287_at | Cluster Incl AA189677 mt70h07 rl Mus musculus cDNA, 5 end/clone = IMAGE-635293/clone_end = 5/gb = AA189677/ | 294.1 | P | 424.1 | P | 301.9 | P |
| | | gi = 1776319/ug = Mm 1873/len = 305/STRA = for | | | | | | |
| 17 | 95343_at | Cluster Incl AB013848 Mus musculus mRNA for peptidylarginine deiminase type I, complete cds/cds = (127,2115)/ | 144.7 | P | 218.3 | P | 228.3 | P |
| | | gb = AB013848/gi = 3845556/ug = Mm 20854/len = 3755/STRA = for | | | | | | |
| 17 | 95356_at | Cluster Incl D00466 Apolipoprotein E/cds = (23,958)/gb = D00466/gi = 220334/ug = Mm.21417/len = 1068/STRA = for | 1894.8 | P | 4136.8 | P | 3737.1 | P |
| 17 | 95368_at | Cluster Incl D86949.Mouse mRNA for plexin 2, complete cds/cds = (579,6233)/gb = D86949/gi = 1655431/ug = Mm 2251/ | 339.8 | P | 369.5 | P | 254.7 | P |
| | | len = 6730/STRA = for | | | | | | |
| 17 | 96564_at | Cluster Incl X54401 Mouse U14 snRNA (intron 5 of hsc70 gene)/cds = (0,155)/gb = X54401/gi = 55071/ug = Mm 2944/ | 3599 | P | 3510.3 | P | 3500.3 | P |
| | | len = 156/STRA = for | | | | | | |
| 17 | 102124_f_at | Cluster Incl AI836879 UI-M-AP0-abg-d-08-0-UI.s2 Mus musculus cDNA, 3 end/clone = UI-M-AP0-abg-d-08-0-UI/ | 431.4 | P | 658.8 | P | 515.6 | P |
| | | clone_end = 3/gb = AI836879/gi = 5471092/ug = Mm 43075/len = 252/STRA = rev | | | | | | |
| 17 | 102141_f_at | Cluster Incl AI462340 ub72a07 x1 Mus musculus cDNA, 3 end/clone = IMAGE-1383252/clone_end = 3/gb = AI462340/ | 344.2 | P | 357.8 | P | 263 | P |
| | | gi = 4316370/ug = Mm 44054/len = 339/STRA = rev | | | | | | |
| 17 | 102171_r_at | Cluster Incl AI132241 ue32f10 x1 Mus musculus cDNA, 3 end/clone = IMAGE-1482091/clone_end = 3/gb = AI132241/ | 800.3 | P | 1010.1 | P | 1000.7 | P |
| | | gi = 3602257/ug = Mm 46845/len = 735/STRA = rev | | | | | | |
| 17 | AFFX- | E Coll BioB 5 prime | 419.1 | P | 569.6 | P | 529.2 | P |
| | BioB-5_at | | | | | | | |
| 17 | AFFX-b- | M12481 Mouse cytoplasmic beta-actin mRNA (_5, _M, _3 represent transcript regions 5 prime, | 76.7 | P | 150.9 | P | 79.1 | A |
| | ActinMur/ | Middle, and 3 prime respectively) | | | | | | |
| | M12481_{—} | | | | | | | |
| | M_st | | | | | | | |
| 17 | 93040_at | Cluster Incl AF091390 Mus musculus phospholemman precursor, gene, complete cds/cds = (0,278)/gb = AF091390/ | 757.4 | P | 851.3 | A | 607.7 | P |
| | | gi = 4206710/ug = Mm.1491/len = 279/STRA = for | | | | | | |
| 17 | 94214_at | Cluster Incl X14961: Fatty acid binding protein heart 1/cds = (21,422)/gb = X14961/gi = 51266/ug = Mm.22220/len = 658/ | 172.6 | P | 233.1 | P | 156.5 | P |
| | | STRA = for | | | | | | |
| 17 | 96904_at | Cluster Incl AI848784: UI-M-AJ1-ahg-c-10-0-Ul.s1 Mus musculus cDNA, 3 end/clone = UI-M-AJ1-ahg-c-10-0-UI/ | 202.1 | P | 330.1 | P | 375 | A |
| | | clone_end = 3/gb = AI848784/gi = 5492690/ug = Mm 30123/len = 490/STRA = for | | | | | | |
| 17 | 98061_at | Cluster Incl AI841192: UI-M-AM0-ad1-e-02-0-UI s1 Mus musculus cDNA, 3 end/clone = UI-M-AM0-adl-e-02-0-UI/ | 320.5 | P | 356.7 | A | 360.8 | P |
| | | clone_end = 3/gb = AI841192/gi = 5475405/ug = Mm 34386/len = 444/STRA = for | | | | | | |
| 17 | 99658_f_at | Cluster Incl AW047907: UI-M-BH1-aln-b-05-0-UI s1 Mus musculus cDNA, 3 end/clone = UI-M-BH1-aln-b-05-0-UI/ | 598.9 | P | 670.7 | P | 497.1 | P |
| | | clone_end = 3/gb = AW047907/gi = 5908436/ug = Mm 43784/len = 398/STRA = for | | | | | | |
| 17 | 101546_at | Cluster Incl AF109906 Mus musculus MHC class III region RD gene, partial cds, Bf, C2, G9A, NG22, G9, HSP70, HSP70, | 183.5 | P | 292.8 | P | 239.7 | P |
| | | HSC70t, and smRNP genes, complete cds, G7A gene, partial cds, and unknown genes/cds = (0,1229)/gb = AF109906/ | | | | | | |
| | | gi = 3986763/ug = Mm 8856/len = 1230/STRA = for | | | | | | |
| 17 | 103913_at | Cluster Incl AI851315 UI-M-BH0-akf-h-11-0-UI s1 Mus musculus cDNA, 3 end/clone = UI-M-BH0-akf-h-11-0-UI/ | 229.1 | P | 281.8 | P | 214.1 | P |
| | | clone_end = 3/gb = AI851315/gi = 5495221/ug = Mm.21435/len = 336/STRA = for | | | | | | |
| 17 | 104349_at | Cluster Incl AA870301: vq14c07 r1 Mus musculus cDNA, 5 end/clone = IMAGE-1094220/clone_end = 5/gb = AA870301/ | 190.7 | P | 136.6 | P | 129.8 | P |
| | | gi = 2965746/ug = Mm.22548/len = 429/STRA = for | | | | | | |
| 17 | 104356_at | Cluster Incl AI465543 mu92f12.x1 Mus musculus cDNA, 3 end/clone = IMAGE-653039/clone_end = 3/gb = AI465543/ | 81.3 | A | 121.9 | P | 105.5 | P |
| | | gi = 4319573/ug = Mm.22581/len = 184/STRA = for | | | | | | |
| 17 | 94365_at | Cluster Incl AA710175 vt49a11 r1 Mus musculus cDNA, 5 end/clone = IMAGE-1166396/clone_end = 5/gb = AA710175/ | 526.4 | P | 441.1 | P | 437.5 | P |
| | | gi = 2720093/ug = Mm.25285/len = 481/STRA = for | | | | | | |
| 17 | 95633_r_at | Cluster Incl AW122653 UI-M-BH2.2-aol-e-11-0-UI s1 Mus musculus cDNA, 3 end/clone = UI-M-BH2 2-aol-e-11-0-UI/ | 462.3 | P | 378.7 | A | 462.3 | P |
| | | clone_end = 3/gb = AW122653/gi = 6098183/ug = Mm 28088/len = 363/STRA = for | | | | | | |
| 17 | 96732_at | Cluster Incl AI851081 UI-M-BH0-akg-a-12-0-UI s1 Mus musculus cDNA, 3 end/clone = UI-M-BH0-akg-a-12-0-UI/ | 249.4 | P | 524.6 | P | 428.6 | P |
| | | clone_end = 3/gb = AI851081/gi = 5494987/ug = Mm 28891/len = 480/STRA = for | | | | | | |
| 17 | 99455_s_at | Cluster Incl D17574 Mouse mRNA for proacrosin-binding protein (sp32), complete cds (alternative splicing product)/ | 279.1 | P | 383.1 | P | 265.5 | P |
| | | cds = (87,1037)/gb = D17574/gi = 498163/ug = Mm 4073/len = 1253/STRA = for | | | | | | |
| 17 | 99505_at | Cluster Incl AI844664 UI-M-AL1-ahr-d-11-0-UI.s1 Mus musculus cDNA, 3 end/clone = UI-M-AL1-ahr-d-11-0-UI/ | 289.2 | P | 309.6 | A | 271.8 | P |
| | | clone_end = 3/gb = AI844664/gi = 5488570/ug = Mm 41750/len = 434/STRA = for | | | | | | |
| 17 | 99527_at | Cluster Incl AB013852 Mus musculus mRNA for Nrf3, complete cds/cds = (237,2219)/gb = AB013852/gi = 4521228/ | 56.5 | P | 150.7 | P | 47.3 | P |
| | | ug = Mm 42138/len = 2544/STRA = for | | | | | | |
| 17 | 100982_at | Cluster Incl AI854539 UI-M-BH0-ajj-e-10-0-UI s1 Mus musculus cDNA, 3 end/clone = UI-M-BHO-ajj-e-10-0-UI/clone_end = 3/ | 103.6 | A | 141.6 | P | 59.4 | P |
| | | gb = AI854539/gi= 5498445/ug = Mm 6720/len = 461/STRA = for | | | | | | |
| 17 | 101397_at | Cluster Incl AA870756 vq26b08 r1 Mus musculus cDNA, 5 end/clone = IMAGE-1095351/clone_end = 5/gb = AA870756/ | 404.6 | P | 460.7 | P | 422.7 | P |
| | | gi = 2966201/ug = Mm 7241/len = 281/STRA = for | | | | | | |
| 17 | 101919_at | Cluster Incl M32309.Zinc finger protein X-linked/cds = (198,2717)/gb = M32309/gi = 202455/ug = Mm 919/len = 6878/ | 147.1 | P | 259 | P | 175.4 | P |
| | | STRA = for | | | | | | |
| 17 | 102193_at | Cluster Incl AA791891 vs54hO4.r1 Mus musculus cDNA, 5 end/clone = IMAGE-1150135/clone_end = 5/gb = AA791891/ | 134.8 | P | 249.4 | P | 151.9 | P |
| | | gi = 2854846/ug = Mm 9821/len = 418/STRA = for | | | | | | |
| 17 | 102196_at | Cluster Incl U37413 Guanine nucleotide binding protein, alpha 11/cds = (57,1136)/gb = U37413/gi = 1051237/ug = Mm 989/ | 110.4 | A | 230.8 | P | 278.1 | P |
| | | len = 1342/STRA = rev | | | | | | |
| 17 | 103315_at | Cluster Incl AW120526 UI-M-BH1-ana-b-09-0-UI s2 Mus musculus cDNA, 3 end/clone = UI-M-BH1-ana-b-09-0-UI/ | 169 | P | 258.7 | P | 138 | P |
| | | clone_end = 3/gb = AW120526/gi = 6095859/ug = Mm 21289/len = 426/STRA = for | | | | | | |
| 17 | 104007_at | Cluster Incl AA986782 uc74c02 x1 Mus musculus cDNA, 3 end/clone = IMAGE-1431362/clone_end = 3/gb = AA986782/ | 227.8 | A | 343.6 | P | 352.2 | P |
| | | gi = 3167784/ug = Mm 26737/len = 394/STRA = rev | | | | | | |
| 17 | 104398_at | Cluster Incl AI846222 UI-M-AM1-agb-g-06-0-UI s1 Mus musculus cDNA, 3 end/clone = UI-M-AM1-agb-g-06-0-UI/ | 107.5 | P | 121.7 | P | 91 | P |
| | | clone_end = 3/gb = AI846222/gi = 5490128/ug = Mm 28655/len = 232/STRA = for | | | | | | |
| 17 | 92330_r_at | Cluster Incl AA517665 vh81f01 r1 Mus musculus cDNA, 3 end/clone = IMAGE-893401/clone_end = 3/gb = AA517665/ | 164.9 | A | 223.6 | P | 291.4 | P |
| | | gi = 2257189/ug = Mm 38891/len = 484/STRA = rev | | | | | | |
| 17 | 92711_at | Cluster Incl M57625 Mast cell protease 6/cds = UNKNOWN/gb = M57625/gi = 200506/ug = Mm 46216/len = 3757/STRA = for | 164.9 | M | 263.7 | P | 208.2 | P |
| 17 | 93130_at | Cluster Incl AW122860 UI-M-BH2.1-aoz-h-09-0-UI s1 Mus musculus cDNA, 3 end/clone = UI-M-BH2 1-aoz-h-09-0-UI/ | 237.8 | A | 281.9 | P | 191.2 | P |
| | | clone_end = 3/gb = AW122860/gi = 6098390/ug = Mm 5117/len = 186/STRA = for | | | | | | |
| 17 | 97706_at | Cluster Incl AA474655 vd53b01.s1 Mus musculus cDNA, 5 end/clone = IMAGE-804265/clone_end = 5/gb = AA474655/ | 91.8 | M | 166.3 | P | 117.8 | P |
| | | gi = 2202810/ug = Mm 38051/len = 305/STRA = for | | | | | | |
| 17 | 99856_r_at | Cluster Incl AI852919 UI-M-BH0-aiz-b-06-0-UI.s1 Mus musculus cDNA, 3 end/clone = UI-M-BH0-aiz-b-06-0-UI/ | 201.4 | A | 276.7 | P | 186.3 | P |
| | | clone_end = 3/gb = AI852919/gi = 5496825/ug = Mm 6680/len = 396/STRA = rev | | | | | | |
| 17 | 101759_at | Cluster Incl D00851: Mus musculus Tcp-1 pseudogene for I-complex polypeptide 1/cds = (0,1352)/gb = D00851/gi = 220608/ | 57.2 | P | 102.3 | P | 60 | P |
| | | ug = Mm 57089/len = 1353/STRA = for | | | | | | |
| 17 | 102583_at | Cluster Incl AJ132195 Mus musculus olfr89 gene, partial/cds = (0,908)/gb = AJ132195/gi = 4186074/ug = Mm 57248/len = 909/ | 196.6 | P | 271.4 | P | 184 | M |
| | | STRA = for | | | | | | |
| 17 | 100751_at | Cluster Incl AF011379 A disintegrin and metalloprotease domain (ADAM) 10/cds = (25,2274)/gb = AF011379/gi = 2282607/ | 184.5 | A | 316.3 | P | 302.5 | P |
| | | ug = Mm 3911/len = 2481/STRA = for | | | | | | |
| 17 | 93311_at | Cluster Incl AF033565: Mus musculus cdc2/CDC28-like protein kinase 3 (Clk3) mRNA, complete cds/cds = (63,1535)/ | −15.6 | A | 220.1 | P | 120.9 | P |
| | | gb = AF033565/gi = 2645653/ug = Mm 18476/len = 1787/STRA = for | | | | | | |
| 17 | 97870_s_at | Cluster Incl AA798624 vy03d11.r1 Mus musculus cDNA, 5 end/clone = IMAGE-1294389/clone_end = 5/gb = AA798624/ | 175.4 | M | 281.9 | P | 115.3 | P |
| | | gi = 2861579/ug = Mm 28339/len = 280/STRA = rev | | | | | | |
| 17 | 98109_at | Cluster Incl AI839526 UI-M-ANO-acn-e-06-0-UI s1 Mus musculus cDNA, 3 end/clone = UI-M-AN0-acn-e-06-0-UI/ | 137.5 | P | 198.3 | P | 156.5 | P |
| | | clone_end = 3/gb = AI839528/gi = 5473741/ug = Mm 34801/len = 355/STRA = for | | | | | | |
| 17 | 99144_s_at | Cluster Incl D50031: Mouse mRNA for TGN38A, complete cds/cds = (26,1087)/gb = D50031/gi = 949828/ug = Mm 4142/ | 131.1 | P | 282.5 | P | 240.8 | P |
| | | len = 1673/STRA = for | | | | | | |
| 17 | 103028_at | Cluster Incl D14042 Mus musculus tlk mRNA for tyrosine kinase, complete cds/cds = (45,1904)/gb = D14042/gi = 220613/ | 180.2 | P | 279.3 | P | 241 | P |
| | | ug = Mm 16009/len = 4224/STRA = for | | | | | | |
| 17 | 103057_at | Cluster Incl AF024570: DNA polymerase delta 1, calalytic domain/cds = (0,3317)/gb = AF024570/gi = 2827902/ | 237.1 | P | 339.8 | P | 342.5 | P |
| | | ug = Mm.16549/len = 3318/STRA = rev | | | | | | |
| 17 | 103914_at | Cluster Incl AI847114: UI-M-AP1-agn-h-04-0-UI s1 Mus musculus cDNA, 3 end/clone = UI-M-AP1-agn-h-04-0-UI/ | 648.9 | P | 676 | P | 477 | P |
| | | clone_end = 3/gb = AI847114/gi = 5491025/ug = Mm.21439/len = 416/STRA = for | | | | | | |
| 17 | 95604_at | Cluster Incl AI843294: UI-M-AO1-ael-f-03-0-UI s1 Mus musculus cDNA, 3 end/clone = UI-M-AO1-aei-f-03-0-UI/ | 424.5 | A | 555.6 | P | 542.9 | P |
| | | clone_end = 3/gb = A1843294/gi = 5477507/ug = Mm 27954/len = 376/STRA = for | | | | | | |
| 17 | 102698_at | Cluster Incl AF045160 Mus musculus hypoxia-inducible factor 1 alpha related factor mRNA, complete cds/cds = (183,2807)/ | 171.6 | P | 242.9 | P | 204 | P |
| | | gb = AF045160/gi = 2895755/ug = Mm.1415/len = 3415/STRA = for | | | | | | |
| 17 | 103438_at | Cluster Incl AF096875 Mus musculus type 2 deiodinase mRNA, complete cds/cds = (154,954)/gb = AF096875/gi = 4206102/ | 83.9 | A | 184.1 | P | 109.4 | P |
| | | ug = Mm 21389/len = 5841/STRA = for | | | | | | |
| 17 | 104480_at | Cluster Incl AI152659 ud93d03 r1 Mus musculus cDNA, 5 end/clone = IMAGE-1478405/clone_end = 5/gb = AI152659/ | 262.1 | P | 361.1 | P | 214.1 | A |
| | | gi = 3681128/ug = Mm.30027/len = 334/STRA = for | | | | | | |
| 17 | 92287_at | Cluster Incl AF033187 Mus musculus SOCS-5 mRNA, complete cds/cds = (0,1610)/gb = AF033187/gi = 2766490/ | 287.8 | P | 442.3 | P | 292.3 | P |
| | | ug = Mm.37204/len = 1611/STRA = for | | | | | | |
| 17 | 92710_at | Cluster Incl U73199 Mus musculus Rho-guanine nucleotide exchange factor mRNA, complete cds/cds = (104,5185)/ | 57.1 | A | 131.1 | P | 70.4 | P |
| | | gb = U73199/gi = 1657834/ug = Mm 4620/len = 5405/STRA = for | | | | | | |
| 17 | 93427_at | Cluster Incl AW122310 UI-M-BH2 2-aos-d-10-0-UI s1 Mus musculus cDNA, 3 end/clone = UI-M-BH2.2-aos-d-10-0-UI/ | 217.5 | P | 244.6 | P | 227.7 | P |
| | | clone_end = 3/gb = AW122310/gi = 6097795/ug = Mm.6493/len = 208/STRA = rev | | | | | | |
| 17 | 94695_at | Cluster Incl C81411 C81411 Mus musculus cDNA, 3 end/clone = J0098H06/clone_end = 3/gb = C81411/gi = 2521741/ | 45.5 | P | 216.9 | P | 99.4 | P |
| | | ug = Mm 13352/len = 586/STRA = rev | | | | | | |
| 17 | 102584_at | Cluster Incl Y18260 Mus musculus mRNA for protein kinase Dyrk1B/cds = (248,2017)/gb = Y18280/gi = 5738713/ | 219.1 | P | 270.5 | P | 284.2 | P |
| | | ug = Mm 57249/len = 2018/STRA = for | | | | | | |
| 17 | 95782_at | Cluster Incl U19033 Melanoma antigen, related sequence 2/cds = (0,992)/gb = U19033/gi = 1165173/ug = Mm 87790/ | 154.5 | P | 218.8 | P | 125.3 | P |
| | | len = 993/STRA = for | | | | | | |
| 17 | 100713_at | Cluster Incl AB020542: Mus musculus mRNA for Zinc finger protein sf 1-6, complete cds/cds = (106,1869)/gb = AB020542/ | 57 | M | 145.9 | P | 71 | P |
| | | gi = 3953592/ug = Mm.26510/len = 2363/STRA = for | | | | | | |
| 17 | 92824_at | Cluster Incl AF051942 Mus musculus type 6 nucleoside diphosphate kinase NM23-M6 (Nm23-M6) mRNA, complete cds/ | 258.5 | A | 371.9 | A | 355 | P |
| | | cds = (121,690)/gb = AF051942/gi = 3228531/ug = Mm 12951/len = 1025/STRA = for | | | | | | |
| 17 | 97490_at | Cluster Incl AJ011145 Mus musculus mRNA for protein encoded by BCL7B gene/cds = (78,623)/gb = AJ011145/ | 62.4 | A | 148.7 | P | 122.2 | P |
| | | gi = 3647321/ug = Mm 31/len = 1546/STRA = for | | | | | | |
| 17 | 103801_at | Cluster Incl AF094761 Mus musculus Rfxank (Rfxank) mRNA, complete cds/cds = (201,1010)/gb = AF094761/gi = 3820617/ | 155.3 | P | 383.6 | A | 242 | P |
| | | ug = Mm 20848/len = 1403/STRA = for | | | | | | |
| 17 | 97433_at | Cluster Incl AJ006590 Mus musculus mRNA for GANP protein/cds = (383,6298)/gb = AJ006590/gi = 4995702/ug = Mm 30098/ | 103.2 | M | 178.9 | A | 243.3 | A |
| | | len = 6429/STRA = for | | | | | | |
| 17 | 103752_r_at | Cluster Incl AW227545 up10e09 x1 Mus musculus cDNA, 3 end/clone = IMAGE-2651656/clone_end = 3/gb = AW227545/ | 86.4 | A | 247.1 | P | 177 | P |
| | | gi = 6556841/ug = Mm 24516/len = 445/STRA = for | | | | | | |
| 17 | 104246_at | Cluster Incl AI852340: UI-M-BH0-ajo-b-02-0-UI s1 Mus musculus cDNA, 3 end/clone = UI-M-BH0-ajo-b-02-0-UI/ | 394.2 | P | 381.4 | A | 383 | P |
| | | clone_end = 3/gb = AI852340/gi = 5496246/ug = Mm 2833/len = 501/STRA = for | | | | | | |
| 17 | 104507_g_at | Cluster Incl AF009328 Mus musculus orphan nuclear hormone receptor isoform mCAR2 mRNA, complete cds/ | 204 | P | 259.1 | M | 247.2 | P |
| | | cds = (244,1104)/gb = AF009328/gi = 2267577/ug = Mm 3077/len = 1332/STRA = for | | | | | | |
| 17 | 96531_at | Cluster Incl AA153773 mr77h06 r1 Mus musculus cDNA, 5 end/clone = IMAGE-603515/clone_end = 5/gb = AA153773/ | 264.9 | M | 312.9 | P | 232.3 | P |
| | | gi = 1724192/ug = Mm 27295/len = 455/STRA = for | | | | | | |
| 17 | 96570_at | Cluster Incl AV381276: AV381276 Mus musculus cDNA, 3 end/clone = 9230111G20/clone_end = 3/gb = AV381276/ | 5.9 | A | 49 | P | 16.8 | A |
| | | gi = 6430353/ug = Mm 29740/len = 165/STRA = for | | | | | | |
| 17 | 96600_at | Cluster Incl C76597 C76597 Mus musculus cDNA, 3 end/clone = J0014E03/clone_end = 3/gb = C76597/gi = 2516927/ | 23.5 | A | 50.5 | P | 43.6 | M |
| | | ug = Mm 31117/len = 601/STRA = rev | | | | | | |
| 17 | 99364_at | Cluster Incl AI661370: va11c04 x1 Mus musculus cDNA, 3 end/clone = IMAGE-722598/clone_end = 3/gb = AI661370/ | 25.7 | P | 42.9 | P | 20.7 | A |
| | | gi = 4764953/ug = Mm 5633/len = 378/STRA = rev | | | | | | |
| 17 | 100730_at | Cluster Incl D37797 Lymphocyte antigen 78/cds = (112,2097)/gb = D37797/gi = 761711/ug = Mm 3300/len = 2620/STRA = for | 107.9 | P | 168.6 | P | 98.3 | M |
| 17 | 92555_at | Cluster Incl AF053454 Mus musculus tetraspan TM4SF (Tspan-6) mRNA, complete cds/cds = (82,819)/gb = AF053454/ | 48.4 | A | 73.6 | A | 57.2 | A |
| | | gi = 2995862/ug = Mm 10180/len = 1058/STRA = for | | | | | | |
| 17 | 98052_at | Cluster Incl AF003999 Mus musculus Golgi SNARE GS15 mRNA, complete cds/cds = (0,335)/gb = AF003999/gi = 2316079/ | 129.7 | M | 303.9 | A | 91.9 | P |
| | | ug = Mm.34315/len = 336/STRA = for | | | | | | |
| 17 | 101501_r_at | Cluster Incl D87973 Imprinted and ancient/cds = (85,1041)/gb = D87973/gi = 4038075/ug = Mm 8154/len = 3442/STRA = for | 56.6 | P | 89.9 | A | 50.3 | A |
| 17 | 94363_at | Cluster Incl AI840815 UI-M-AH0-adb-c-03-0-UI s1 Mus musculus cDNA, 3 end/clone = UI-M-AH0-adb-c-03-0-UI/ | 62.1 | A | 140.1 | A | 182.8 | P |
| | | clone_end = 3/gb = AI840815/gi = 5475028/ug = Mm.25269/len = 363/STRA = for | | | | | | |
| 17 | 94990_at | Cluster Incl AI854725 UI-M-BHO-akc-g-05-0-UI.s1 Mus musculus cDNA, 3 end/clone = UI-M-BH0-akc-g-05-0-UI/ | −8.2 | P | 119 | A | 172.7 | P |
| | | clone_end = 3/gb = AI854725/gi = 5498631/ug = Mm 27312/len = 512/STRA = for | | | | | | |
| 17 | 96759_r_at | Cluster Incl AW061265: UI-M-BH1-amj-f-10-0-UI s2 Mus musculus cDNA, 3 end/clone = UI-M-BH1-amj-f-10-0-UI/ | 371.6 | P | 505.7 | A | 359.5 | A |
| | | clone_end = 3/gb = AW061265/gi = 6009016/ug = Mm 28994/len = 445/STRA = for | | | | | | |
| 17 | 98965_at | Cluster Incl AC002397: Mouse chromosome 6 BAC-284H12 (Research Genetics mouse BAC library) complete sequence/ | 21.7 | A | 321.6 | P | 328.8 | P |
| | | cds = (1,1323)/gb = AC002397/gi = 3287367/ug = Mm 3634/len = 1741/STRA = rev | | | | | | |
| 17 | 99537_at | Cluster Incl AF100694 Mus musculus Pontin52 mRNA, complete cds/cds = (144,15l4)/gb = AF100694/gi = 4106527/ | 22.9 | A | 247 | A | 260.9 | P |
| | | ug = Mm 42195/len = 1709/STRA = for | | | | | | |
| 17 | 101873_at | Cluster Incl X95603: M musculus insl3 gene/cds = (3,368)/gb = X95603/gi = 1771291/ug = Mm 87963/len = 583/STRA = for | 69.1 | P | 88 | A | 89.1 | A |
| 17 | 103946_at | Cluster Incl U87814 Mus musculus PEST phosphatase Interacting protein mRNA, complete cds/cds = (273,1520)/ | 36.3 | A | 98.4 | A | 113 | P |
| | | gb = U87814/gi = 1857711/ug = Mm 2534/len = 1692/STRA = for | | | | | | |
| 17 | 92246_at | Cluster Incl D85925 Mus musculus mRNA for anagen-specific protein mKAP13, complete cds/cds = (45,548)/gb = D85925/ | 167.5 | M | 191.1 | P | 109 | P |
| | | gi = 1944386/ug = Mm 3502/len = 844/STRA = for | | | | | | |
| 17 | 93676_at | Cluster Incl U93583: RAD51 associated protein 1/cds = (43,1056)/gb = U93583/gi = 2697100/ug = Mm.8156/len = 1165/ | 361.6 | P | 377.5 | M | 305.6 | M |
| | | STRA = for | | | | | | |
| 17 | 93933_at | Cluster Incl U89924: Mus musculus protein phosphatase 1 binding protein PTG mRNA, complete cds/cds = (60,944)/ | 275.6 | A | 341.2 | P | 126.9 | A |
| | | gb = U89924/gi = 1888565/ug = Mm 89946/len = 1169/STRA = for | | | | | | |
| 17 | 95369_at | Cluster Incl AF021345 Mus musculus plasma selenoprotein P (SELP) gene, complete cds/cds = (56,1198)/gb = AF021345/ | 61.6 | A | 108.5 | P | 64.9 | P |
| | | gi = 4103141/ug = Mm 22699/len = 2823/STRA = for | | | | | | |
| 17 | 95955_at | Cluster Incl AI606577 vb90e10 y1 Mus musculus cDNA, 5 end/clone = IMAGE-764298/clone_end = 5/gb = AI606577/ | 53.1 | A | 24.2 | M | 51.4 | M |
| | | gi = 4615744/ug = Mm.24932/len = 543/STRA = for | | | | | | |
| 17 | 98367_at | Cluster Incl X73572 M musculus Hoxd-3 gene, complete CDS/cds = (218,865)/gb = X73572/gi = 313248/ug = Mm 4432/ | 16.5 | A | 83.1 | P | 40.4 | A |
| | | len = 2128/STRA = for | | | | | | |
| 17 | 101157_at | Cluster Incl V00790 Part of messenger RNA for mouse delta-immunoglobulin (codes for part of exon 8 —one of two | 139.8 | A | 220.7 | P | 331.4 | P |
| | | alternate C-termini)/cds = (0,82)/gb = V00790/gi = 51778/ug = Mm.4939/len = 206/STRA = for | | | | | | |
| 17 | 95670_at | Cluster Incl AI639868: UI-M-AH0-acw-a-05-0-UI s1 Mus musculus cDNA, 3 end/clone = UI-M-AH0-acw-a-05-0-UI/ | 121.7 | P | 158.9 | A | 131.2 | A |
| | | clone_end = 3/gb = AI839868/gi = 5474081/ug = Mm 29580/len = 406/STRA = for | | | | | | |
| 17 | 101076_r_at | Cluster Incl AB016592 Mus musculus mRNA for GOB-4, complete cds/cds = (51,578)/gb = AB016592/gi = 3395165/ | 57.2 | M | 67.5 | A | 63.6 | P |
| | | ug = Mm.7244/len = 758/STRA = for | | | | | | |
| 17 | 102352_at | Cluster Incl AW121380.UI-M-BH2 2-aom-f-06-0-UI s1 Mus musculus cDNA, 3 end/clone = UI-M-BH2 2-aom-f-06-0-UI/ | 72.3 | P | 118 | M | 51.5 | A |
| | | clone_end = 3/gb = AW121380/gi = 6096713/ug = Mm.11360/len = 526/STRA = rev | | | | | | |
| 17 | 101908_s_at | Cluster Incl AF101164 Mus musculus biliary glycoprotein 2 long isoform (Bgp2) mRNA, complete cds/cds = υ,1120)/ | 60.7 | A | 166.2 | P | 15.9 | M |
| | | gb = AF101164/gi = 4092740/ug = Mm 89819/len = 1321/STRA = for | | | | | | |
| 17 | 102217_at | Cluster Incl AI639925 vr74g08 x1 Mus musculus cDNA, 3 end/clone = IMAGE-1134494/clone_end = 3/gb = AI639925/ | 87.4 | P | 116.8 | A | 123.6 | A |
| | | gi = 4703034/ug = Mm 10188/len = 484/STRA = for | | | | | | |
| 17 | 102240_at | Cluster Incl AF049330: Mus musculus PPAR gamma coactivator (PGC-1) mRNA, complete cds/cds = (91,2484)/ | 40.7 | A | 81.9 | P | 80 | P |
| | | gb = AF049330/gi = 3046930/ug = Mm 10707/len = 3029/STRA = for | | | | | | |
| 17 | 103759_at | Cluster Incl D31788: BP-3 alloantigen/cds = (13,948)/gb = D31788/gi = 559368/ug = Mm.24612/len = 1255/STRA = for | 45.5 | A | 56.7 | A | 33.4 | A |
| 17 | 92507_at | Cluster Incl Y12229 Utrophin/cds = (0,10289)/gb = Y12229/gi = 1934962/ug = Mm.42222/len = 11096/STRA = for | 21.6 | P | 94.9 | M | 26.8 | A |
| 17 | 93923_at | Cluster Incl AW259620 up31b08 x1 Mus musculus cDNA, 3 end/clone = IMAGE-2655927/clone_end = 3/gb = AW259620/ | −70.3 | P | 443.3 | P | 574.2 | A |
| | | gi = 6632601/ug = Mm.89860/1en = 176/STRA = for | | | | | | |
| 17 | 94124_at | Cluster Incl AB006074 Mus musculus mTOP3 mRNA for topoisomerase III, complete cds/cds = (265,3276)/gb = AB006074/ | 575.1 | A | 807.7 | A | 693.6 | A |
| | | gi = 3061307/ug = Mm 10708/len = 3741/STRA = for | | | | | | |
| 17 | 95800_s_at | Cluster Incl X53250 Zinc finger protein, autosomal/cds = (0,2228)/gb = X53250/gi = 55468/ug = Mm 87837/len = 2229/ | 28.2 | P | 56.9 | P | 33.5 | M |
| | | STRA = for | | | | | | |
| 17 | 96414_at | Cluster Incl D50656 Mus musculus mRNA for non-selective cation channel, complete cds/cds = (288,1560)/gb = D50656/ | −16.2 | P | 83.7 | P | 32.2 | A |
| | | gi = 2924621/ug = Mm.10098/len = 2785/STRA = for | | | | | | |
| 17 | 102026_s_at | Cluster Incl AB011000 Mus musculus mRNA for choline/ethanolamine kinase, complete cds/cds = (299,1483)/ | 166.3 | A | 299.6 | A | 158.9 | A |
| | | gb = AB011000/gi = 2897728/ug = Mm.10124/len = 1708/STRA = for | | | | | | |
| 17 | 102042_at | Cluster Incl AI842308: UI-M-AM1-afz-a-02-0-Ul.s1 Mus musculus cDNA, 3 end/clone = UI-M-AM1-afz-a-02-0-UI/ | 127.5 | A | 192.9 | A | 123.4 | A |
| | | clone_end = 3/gb = AI842308/gi = 5476486/ug = Mm 10200/len = 475/STRA = for | | | | | | |
| 17 | 103663_at | Cluster Incl AI837330 UI-M-AK0-adf-d-04-0-UI s1 Mus musculus cDNA, 3 end/clone = UI-M-AK0-adt-d-04-0-UI/ | 115.1 | A | 89.8 | A | 147.3 | A |
| | | clone_end = 3/gb = AI837330/gi = 5471543/ug = Mm 2069/len = 332/STRA = for | | | | | | |
| 17 | 95551_at | Cluster Incl AI849232 UI-M-AJ1-agz-c-07-0-UI.s1 Mus musculus cDNA, 3 end/clone = UI-M-AJ1-agz-c-07-0-UI/ | 100 | A | 212.2 | A | 159.8 | P |
| | | clone_end = 3/gb = AI849232/gi = 5493138/ug = Mm.27738/len = 419/STRA = for | | | | | | |
| 17 | 104017_at | Cluster Incl AB033887 Mus musculus mACS4 vanant2 mRNA for Acyl-CoA synthetase 4 vanant2, complete cds/ | 3.5 | M | 44.9 | A | 6 | A |
| | | cds = (317,2452)/gb = AB033887/gi = 6172344/ug = Mm 26894/len = 2495/STRA = for | | | | | | |
| 17 | 92344_at | Cluster Incl AF010600 Mus musculus TNF-response element binding protein mRNA, complete cds/cds = (177,2702)/ | −16.2 | A | 20.8 | A | 19.1 | A |
| | | gb = AF010600/gi = 2275596/ug = Mm.3909/len = 3064/STRA = for | | | | | | |
| 17 | 92993_r_at | Cluster Incl AI324972: mm78d11 x1 Mus musculus cDNA, 3 end/clone = IMAGE-534549/clone_end = 3/gb = AI324972/ | 32.7 | P | 31.3 | M | 15.2 | A |
| | | gi = 4059401/ug = Mm 5073/len = 431/STRA = rev | | | | | | |
| 17 | 96539_at | Cluster Incl AW212071 uo81h11.x1 Mus musculus cDNA, 3 end/clone = IMAGE-2648997/clone_end = 3/gb = AW212071/ | −54.6 | A | −17.2 | M | 24.6 | A |
| | | gi = 6518072/ug = Mm 27699/len = 402/STRA = for | | | | | | |
| 17 | 97100_at | Cluster Incl AI854248 UI-M-BH0-ajz-h-11-0-UI.s1 Mus musculus cDNA, 3 end/clone = UI-M-BH0-ajz-h-11-0-UI/ | 79.3 | P | 93.5 | P | −69 | A |
| | | clone_end = 3/gb = AI854248/gi = 5498154/ug = Mm.32074/len = 516/STRA = rev | | | | | | |
| 17 | 100739_at | Cluster Incl U96702.Mus musculus serine proteinase inhibitor mBM17 mRNA, partial cds/cds = (0,440)/gb = U96702/ | 36.8 | A | 52.4 | A | −6.2 | A |
| | | gi = 2104736/ug = Mm 3518/len = 594/STRA = for | | | | | | |
| 18 | 92634_at | Cluster Incl U12620: Dipeptidylpeptidase 4/cds = (117,2399)/gb = U12620/gi = 1065904/ug = Mm.1151/len = 4943/STRA = for | 304.4 | P | 305 | P | 221.2 | P |
| 18 | 92643_at | Cluster Incl X74671 Neurofibromatosis 2/cds = (576,2363)/gb = X74671/gi = 456656/ug = Mm.1190/len = 2604/STRA = for | 390.6 | P | 511 | P | 351.3 | P |
| 18 | 92882_at | Cluster Incl Y00094 Mouse mRNA for ras-related YPT1 protein/cds = (129,746)/gb = Y00094/gi = 55456/ug = Mm 14530/ | 667.2 | P | 864.2 | P | 460.4 | P |
| | | len = 1428/STRA = for | | | | | | |
| 18 | 93090_at | Cluster Incl M23362: Fibroblast growth factor receptor 2/cds = (0,1037)/gb = M23362/gi = 533219/ug = Mm.16340/len = 1038/ | 1395.5 | P | 1421.6 | P | 1086.4 | P |
| | | STRA = for | | | | | | |
| 18 | 93341_r_at | Cluster Incl AF043120 Mus musculus beta prime coatomer protein mRNA, partial cds/cds = (0,1352)/gb = AF043120/ | 1093.8 | P | 1258.9 | P | 1013.3 | P |
| | | gi = 2809536/ug = Mm.18729/1en = 1615/STRA = for | | | | | | |
| 18 | 93509_at | Cluster Incl U57690: Ubiqultin-conjugating enzyme E2B (RAD6 homology)/cds = (43,501)/gb = U57690/gi = 1373352/ | 282.2 | P | 226.5 | P | 164.2 | P |
| | | ug = Mm 1920/1en = 923/STRA = for | | | | | | |
| 18 | 93841_at | Cluster Incl AW060249 UI-M-BH1-amv-e-10-0-UI s1 Mus musculus cDNA, 3 end/clone = UI-M-BH1-amv-e-10-0-UI/ | 450.7 | P | 557.9 | P | 472.5 | P |
| | | clone_end = 3/gb = AW060249/gi = 6008040/ug = Mm 21617/len = 553/STRA = for | | | | | | |
| 18 | 94223_at | Cluster Incl AJ010045: Mus musculus mRNA for Rho guanine nucleotide-exchange factor, splice variant NET1A/ | 1595.2 | P | 1702.6 | P | 1376.1 | P |
| | | cds = (159,1784)/gb = AJ010045/gi = 4138206/ug = Mm 22261/len = 3290/STRA = for | | | | | | |
| 18 | 94249_at | Cluster Incl AW226968: um64d03 y1 Mus musculus cDNA, 5 end/clone = IMAGE-2286149/clone_end = 5/gb = AW226968/ | 465.8 | P | 563.8 | P | 461.9 | P |
| | | gi = 6556264/ug = Mm 22374/len = 425/STRA = for | | | | | | |
| 18 | 94882_at | Cluster Incl AA684506: vr02a10.r1 Mus musculus cDNA, 5 end/clone = IMAGE-1110618/clone_end = 5/gb = AA684506/ | 346.2 | P | 498.4 | P | 206.2 | P |
| | | gi = 2670223/ug = Mm.23877/len = 487/STRA = rev | | | | | | |
| 18 | 95415_f_at | Cluster Incl AI132585 ue97d10 x1 Mus musculus cDNA, 3 end/clone = IMAGE-1499059/clone_end = 3/gb = AI132585/ | 545.9 | P | 590.4 | P | 476.1 | P |
| | | gi = 3602592/ug = Mm.24276/len = 597/STRA = for | | | | | | |
| 18 | 96610_at | Cluster Incl AW046442 UI-M-BH1-akp-e-03-0-UI s1 Mus musculus cDNA, 3 end/clone = UI-M-BH1-akp-e-03-0-UI/ | 598.9 | P | 709.7 | P | 594.3 | P |
| | | clone_end = 3/gb = AW046442/gi = 5906971/ug = Mm.27082/len = 451/STRA = for | | | | | | |
| 18 | 96625_at | Cluster Incl AW123132 UI-M-BH2 1-apd-a-04-0-UI s1 Mus musculus cDNA, 3 end/clone = UI-M-BH2 1-apd-a-04-0-UI/ | 373.2 | P | 369.7 | P | 313.2 | P |
| | | clone_end = 3/gb = AW123132/gi = 6098882/ug = Mm 2718/len = 488/STRA = for | | | | | | |
| 18 | 96669_at | Cluster Incl AW122973 UI-M-BH2.1-apb-a-02-0-UI s1 Mus musculus cDNA, 3 end/clone = UI-M-BH2 1-apb-a-02-0-UI/ | 336.2 | P | 662.4 | P | 415.2 | P |
| | | clone_end = 3/gb = AW122973/gi = 6098503/ug = Mm 27394/len = 467/STRA = for | | | | | | |
| 18 | 96302_at | Cluster Incl AA711516 vu26g06 r1 Mus musculus cDNA, 5 end/clone = IMAGE-1181818/clone_end = 5/gb = AA711516/ | 688.6 | P | 614 | P | 481.9 | P |
| | | gi = 2721434/ug = Mm 29915/len = 260/STRA = rev | | | | | | |
| 18 | 96879_at | Cluster Incl AI852338 UI-M-BH0-ajo-a-12-0-UI s1 Mus musculus cDNA, 3 end/clone = UI-M-BH0-ajo-a-12-0-UI/ | 1463 | P | 1401.2 | P | 1089.7 | P |
| | | clone_end = 3/gb = AI852338/gi = 5496244/ug = Mm.30074/len = 475/STRA = for | | | | | | |
| 18 | 97442_at | Cluster Incl AW124340 UI-M-BH2 1-apq-f-11-0-UI s1 Mus musculus cDNA, 3 end/clone = UI-M-BH2 1-apq-f-11-0-UI/ | 401.1 | P | 858 | P | 544.5 | P |
| | | clone_end = 3/gb = AW124340/gi = 6099870/ug = Mm 30239/len = 224/STRA = for | | | | | | |
| 18 | 98055_at | Cluster Incl AW121500 UI-M-BH2.2-aop-a-10-0-UI s1 Mus musculus cDNA, 3 end/clone = UI-M-BH2 2-aop-a-10-0-UI/ | 267.8 | P | 348.1 | P | 263.6 | P |
| | | clone_end = 3/gb = AW121500/gi = 6096833/ug = Mm 34330/len = 384/STRA = for | | | | | | |
| 18 | 98150_at | Cluster Incl L26528 RAB11B, member RAS oncogene family/cds = (35,691)/gb = L26528/gi = 433063/ug = Mm 35727/ | 540.9 | P | 654.3 | P | 497.6 | P |
| | | len = 1763/STRA = for | | | | | | |
| 18 | 98151_s_at | Cluster Incl Z17804 Catenin src/cds = (342,3077)/gb = Z17804/gi = 53544/ug = Mm 35738/len = 4636/STRA = rev | 647.7 | P | 820.3 | P | 521.3 | P |
| 18 | 99119_at | Cluster Incl D00472 Cofilin 1, non-muscle/cds = (94,594)/gb = D00472/gi = 220383/ug = Mm 4024/len = 1134/STRA = for | 3807.2 | P | 4032.1 | P | 3743.7 | P |
| 18 | 99639_at | Cluster Incl D84096 Mouse mRNA for ubiquitin carboxyl-terminal hydrolase (UBP), complete cds/cds = (67,2448)/ | 213.4 | P | 414.9 | P | 277.6 | P |
| | | gb = D84096/gi = 1902990/ug = Mm 4355/len = 3236/STRA = for | | | | | | |
| 18 | 99650_at | Cluster Incl X90945 M. musculus mRNA for casein kinase I-alpha/cds = (0,85)/gb = X90945/gi = 975690/ug = Mm.43737/ | 1937.9 | P | 2431.7 | P | 1805 | P |
| | | len = 342/STRA = for | | | | | | |
| 18 | 100069_at | Cluster Incl M77497 Cytochrome P450, 2f2/cds = (61,1536)/gb = M77497/gi = 192927/ug = Mm 4515/len = 1810/STRA = for | 497.8 | P | 544.8 | P | 500.4 | P |
| 18 | 100553_at | Cluster Incl L46855 Ret finger protein/cds = (148,1716)/gb = L46855/gi = 1161121/ug = Mm 55/len = 1950/STRA = for | 689.3 | P | 676.2 | P | 605.4 | P |
| 18 | 100616_at | Cluster Incl AF012710.Mus musculus centromere protein A (Cenp-a) gene, promoter region and partial cds/cds = (148,210)/ | 399.3 | P | 628.6 | P | 348.7 | P |
| | | gb = AF012710/gi = 2465204/ug = Mm.6579/len = 211/STRA = for | | | | | | |
| 18 | 101050_at | Cluster Incl AF031381 Mus musculus KOI-4 gene, partial cds/cds = (0,130)/gb = AF031381/gi = 2623677/ug = Mm.7013/ | 904.6 | P | 1050.8 | P | 846.4 | P |
| | | len = 341/STRA = for | | | | | | |
| 18 | 101073_at | Cluster Incl X67469 Low density lipoprotein receptor related protein/cds = (441,14078)/gb = X67469/gi = 49941/ug = Mm 7221/ | 1101.3 | P | 1404.5 | P | 1301 | P |
| | | len = 14849/STRA = for | | | | | | |
| 18 | 101536_at | Cluster Incl U35312 Mus musculus nuclear receptor co-repressor mRNA, complete cds/cds = (116,7477)/gb = U35312/ | 240.2 | P | 460 | P | 297.2 | P |
| | | gi = 1022717/ug = Mm 88061/len = 7780/STRA = for | | | | | | |
| 18 | 101555_at | Cluster Incl X57277 RAS-related C3 botulinum substrate 1/cds = (140,718)/gb = X57277/gi = 53885/ug = Mm 889/len = 886/ | 3559.6 | P | 3946.4 | P | 3636.5 | P |
| | | STRA = for | | | | | | |
| 18 | 102333_at | Cluster Incl AF057286 Mus musculus intersectin-EH binding protein Ibp2 mRNA, partial cds/cds = (0,1530)/gb = AF057286/ | 749.7 | P | 881.5 | P | 617.6 | P |
| | | gi = 3063648/ug = Mm 10754/len = 2322/STRA = for | | | | | | |
| 18 | 102767_at | Cluster Incl AW121700 UI-M-BH2 3-aoc-g-01-0-UI s1 Mus musculus cDNA, 3 end/clone = UI-M-BH2,3-aoc-g-01-0-UI/ | 995.6 | P | 1277.8 | P | 1069.7 | P |
| | | clone_end = 3/gb = AW121700/gi = 6097033/ug = Mm 13080/len = 387/STRA = for | | | | | | |
| 18 | 103020_s_at | Cluster Incl AI317205 ui35g09 y1 Mus musculus cDNA, 5 end/clone = IMAGE-1853344/clone_end = 5/gb = AI317205/ | 387.4 | P | 927 | P | 623.9 | P |
| | | gi = 4032472/ug = Mm 15918/len = 235/STRA = for | | | | | | |
| 18 | 103029_at | Cluster Incl D86344 Mouse mRNA for Topoisomerase-lnhibitor suppressed, complete cds/cds = (86,1495)/gb = D86344/ | 768.1 | P | 1328.6 | P | 573.8 | P |
| | | gi = 1711237/ug = Mm 1605/len = 2196/STRA = for | | | | | | |
| 18 | 103073_i_at | Cluster Incl AI842969.UI-M-AO1-aem-g-05-0-UI s1 Mus musculus cDNA, 3 end/clone = UI-M-AO1-aem-g-05-0-UI/ | 876.8 | P | 1307.2 | P | 905.6 | P |
| | | clone_end = 3/gb = AI842969/gi = 5477182/ug = Mm 16976/len = 340/STRA = for | | | | | | |
| 18 | 103099_f_at | Cluster Incl AI663971 ui16h11 r1 Mus musculus cDNA, 5 end/clone = IMAGE-1851525/clone_end = 5/gb = AI663971/ | 1519.4 | P | 1692.6 | P | 1300.1 | P |
| | | gi = 4767554/ug = Mm 17537/len = 400/STRA = for | | | | | | |
| 18 | 103435_at | Cluster Incl AW045910 UI-M-BH1-aku-c-03-0-UI s1 Mus musculus cDNA, 3 end/clone = UI-M-BH1-aku-c-03-0-UI/ | 1221.4 | P | 1399.9 | P | 1012.5 | P |
| | | clone_end = 3/gb = AW045910/gi = 5906439/ug = Mm 19073/len = 454/STRA = for | | | | | | |
| 18 | 103597_at | Cluster Incl X63349 M musculus tyrp2 mRNA for tyrosinase-related protein-2/cds = (404,1957)/gb = X63349/gi = 55065/ | 1254.4 | P | 1360.7 | P | 1005.2 | P |
| | | ug = Mm 19987/len = 2182/STRA = for | | | | | | |
| 18 | 103810_at | Cluster Incl AB010152 Mus musculus mRNA for p73H, complete cds/cds = (118,1878)/gb = AB010152/gi = 3445481/ | 863.8 | P | 1108.5 | P | 812.2 | P |
| | | ug = Mm 20894/len = 4669/STRA = for | | | | | | |
| 18 | 103816_at | Cluster Incl U89915: Mus musculus junctional adhesion molecule (Jam) mRNA, complete cds/cds = (70,972)/gb = U89915/ | 926.8 | P | 1102.9 | P | 744.7 | P |
| | | gi = 3462454/ug = Mm.20903/len = 2029/STRA = for | | | | | | |
| 18 | 103855_at | Cluster Incl AW123286 UI-M-BH2 1-apg-g-06-0-UI S1 Mus musculus cDNA, 3 end/clone = UI-M-BH2 1-apg-g-06-0-UI/ | 507.3 | P | 871.3 | P | 706.8 | P |
| | | clone_end = 3/gb = AW123286/gi = 6098816/ug = Mm 21072/len = 368/STRA = for | | | | | | |
| 18 | 104625_at | Cluster Incl AA874130 vw87g02 r1 Mus musculus cDNA, 5 end/clone = IMAGE-1261970/clone_end = 5/gb = AA874130/ | 658.6 | P | 770.3 | P | 719.4 | P |
| | | gi = 2978819/ug = Mm 24265/len = 204/STRA = for | | | | | | |
| 18 | 94375_at | Cluster Incl Y11666 M musculus gene encoding hexokinase II, exon 1 (and joined CDS)/cds = (479,3232)/gb = Y11666/ | 258.2 | P | 537.2 | P | 319.4 | P |
| | | gi = 1907082/ug = Mm.2549/len = 5496/STRA = for | | | | | | |
| 18 | 96185_at | Cluster Incl AB004305 Adaptor-related protein complex AP-3, delta subunit/cds = (220,3819)/gb = AB004305/gi = 2828340 | 973.2 | P | 988.3 | P | 881.4 | P |
| | | ug = Mm 28463/len = 4730/STRA = rev | | | | | | |
| 18 | 96818_at | Cluster Incl AA762522 vv84d02 r1 Mus musculus cDNA, 5 end/clone = IMAGE-1229091/clone_end = 5/gb = AA762522/ | 396.2 | P | 425.8 | P | 354.4 | P |
| | | gi = 2812269/ug = Mm.29343/len = 523/STRA = for | | | | | | |
| 18 | 97823_at | Cluster Incl AW046532 UI-M-BH1-akz-g-10-0-UI s1 Mus musculus cDNA, 3 end/clone = UI-M-BH1-akz-g-10-0-UI/ | 464.9 | P | 673.9 | P | 501.9 | P |
| | | clone/end = 3/gb = AW046532/gi = 5907061/ug = Mm 30167/len = 509/STRA = for | | | | | | |
| 18 | 97933_at | Cluster Incl AW045317: UI-M-BH1-akn-g-06-0-UI.s1 Mus musculus cDNA, 3 end/clone = UI-M-BH1-akn-g-06-0-UI/ | 654.4 | P | 854.6 | P | 795.2 | P |
| | | clone_end = 3/gb = AW045317/gi = 5905846/ug = Mm.30248/len = 324/STRA = for | | | | | | |
| 18 | 97951_s_at | Cluster Incl U37775 Tuberous sclerosis 2/cds = (34,5259)/gb = U37775/gi = 1181664/ug = Mm 30435/len = 5351/STRA = for | 398.3 | P | 459.1 | P | 359.7 | P |
| 18 | 97989_at | Cluster Incl M81483 Mus musculus calmodulin dependent phosphatase catalytic subunit (Cam-Prp) mRNA, 3 end/ | 288.3 | P | 393 | P | 342.5 | P |
| | | cds = (0,1547)/gb = M81483/gi = 192537/ug = Mm.3150/len = 1800/STRA = rev | | | | | | |
| 18 | 98033_at | Cluster Incl AA710132: vt45a05 r1 Mus musculus cDNA, 5 end/clone = IMAGE-1166000/clone_end = 5/gb = AA710132/ | 1203 | P | 1400.5 | P | 1111.4 | P |
| | | gi = 2720050/ug = Mm 3311/len = 339/STRA = for | | | | | | |
| 18 | 98424_at | Cluster Incl D83966 Protein tyrosine phosphatase, non-receptor type 13/cds = (41,7393)/gb = D83966/gi = 1232103/ | 555.7 | P | 683.2 | P | 559.7 | P |
| | | ug = Mm 3414/len = 7912/STRA = rev | | | | | | |
| 18 | 98975_at | Cluster Incl AI019999 ua95g09 r1 Mus musculus cDNA, 5 end/clone = IMAGE-1365280/clone_end = 5/gb = AI019999/ | 299.5 | P | 472.2 | P | 283.2 | P |
| | | gi = 3234335/ug = Mm 36725/len = 486/STRA = for | | | | | | |
| 18 | 99025_at | Cluster Incl L25125 Mouse RNA helicase and RNA-dependent ATPase from the DEAD box family mRNA, complete cds/ | 402.7 | P | 764.9 | P | 509.9 | P |
| | | cds = (97,1533)/gb = L25125/gi = 407991/ug = Mm 3880/len = 2552/STRA = rev | | | | | | |
| 18 | 99920_at | Cluster Incl U65586: Telomeric repeat binding factor 1/cds = (12,1277)/gb = U65586/gi = 1655898/ug = Mm 4306/len = 1628/ | 57.1 | P | 129.8 | P | 74.9 | P |
| | | STRA = rev | | | | | | |
| 18 | 100887_at | Cluster Incl AI838562 UI-M-AO0-abz-a-04-0-UI s1 Mus musculus cDNA, 3 end/clone = UI-M-AO0-abz-a-04-0-UI/ | 257.7 | P | 374.1 | P | 211.3 | P |
| | | clone_end = 3/gb = AI838562/gi = 5472775/ug = Mm 5383/len = 252/STRA = for | | | | | | |
| 18 | 100889_at | Cluster Incl AI838576 UI-M-AO0-abz-c-02-0-UI s1 Mus musculus cDNA, 3 end/clone = UI-M-AO0-abz-c-02-0-UI/ | 798.9 | P | 792.7 | P | 584.2 | P |
| | | clone_end = 3/gb = AI838576/gi = 5472789/ug = Mm 54120/len = 161/STRA = rev | | | | | | |
| 18 | 100915_at | Cluster Incl AW125698 UI-M-BH2 2-aqi-f-10-0-UI s1 Mus musculus cDNA, 3 end/clone = UI-M-BH2 2-aqi-f-10-0-UI/ | 2509.2 | P | 3437.6 | P | 2532.2 | P |
| | | clone_end = 3/gb = AW125698/gi = 6101228/ug = Mm.5938/len = 128/STRA = for | | | | | | |
| 18 | 101389_at | Cluster Incl AB008553 Mus musculus mRNA for mLGP85/LIMP II, complete cds/cds = (205,1641)/gb = AB008553/ | 361 | P | 372 | P | 282.5 | P |
| | | gi = 2618485/ug = Mm.7176/len = 1853/STRA = for | | | | | | |
| 18 | 101851_at | Cluster Incl AF029215 Antigen Identified by monoclonal antibody MRC OX-2/cds = (0,836)/gb = AF029215/gi = 2739125/ | 326.6 | P | 453.4 | P | 326.5 | P |
| | | ug = Mm 8649/len = 837/STRA = for | | | | | | |
| 18 | 102211_r_at | Cluster Incl U66835: Mus musculus unknown protein mRNA, partial cds/cds = (0,98)/gb = U66835/gi = 2970116/ | 289.2 | P | 352.2 | P | 167.2 | P |
| | | ug = Mm 10102/len = 323/STRA = for | | | | | | |
| 18 | 102670_at | Cluster Incl AI848706 UI-M-AH1-agv-h-09-0-UI s1 Mus musculus cDNA, 3 end/clone = UI-M-AH1-agv-b-09-0-UI/ | 335.6 | P | 416.4 | P | 275.7 | P |
| | | clone_end = 3/gb = AI848706/gi = 5492612/ug = Mm.13758/len = 542/STRA = for | | | | | | |
| 18 | 102930_at | Cluster Incl U14133: Silver/cds = (0,1880)/gb = U14133/gi = 887940/ug = Mm 16756/len = 1881/STRA = for | 924.6 | P | 1397.9 | P | 800.9 | P |
| 18 | 103443_at | Cluster Incl AA711704 vu58c09 r1 Mus musculus cDNA, 5 end/clone = IMAGE-1195600/clone_end = 5/gb = AA711704/ | 1736.1 | P | 2072.1 | P | 1486.8 | P |
| | | gi = 2721622/ug = Mm.21476/len = 249/STRA = for | | | | | | |
| 18 | 103783_at | Cluster Incl AI648965 uk33d12.x1 Mus musculus cDNA, 3 end/clone = lMAGE-1970807/clone_end = 3/gb = AI648965/ | 242.3 | P | 504.7 | P | 289.2 | P |
| | | gi = 4729799/ug = Mm.24893/len = 509/STRA = rev | | | | | | |
| 18 | 104244_at | Cluster Incl AI606891 vw20f11 x1 Mus musculus cDNA, 3 end/clone = IMAGE-1244397/clone_end = 3/gb = AI606891/ | 483.7 | P | 537.6 | P | 337.1 | P |
| | | gi = 4616058/ug = Mm 28210/len = 322/STRA = for | | | | | | |
| 18 | 104248_at | Cluster Incl AW227650 up12b09 y1 Mus musculus cDNA, 5 end/clone = IMAGE-2654105/clone_end = 5/gb = AW227650/ | 785.5 | P | 1068.4 | P | 632.1 | P |
| | | gi = 6556946/ug = Mm 28345/len = 440/STRA = for | | | | | | |
| 18 | 104437_at | Cluster Incl Z30174: Zinc finger protein 30/cds = (143,1789)/gb = Z30174/gi = 456268/ug = Mm 2927/len = 2604/STRA = for | 580.7 | P | 601.6 | P | 320.5 | P |
| 18 | 92262_at | Cluster Incl AF012923 Mus musculus p53-inducible zinc finger protein (Wig-1) mRNA, complete cds/cds = (173,1045)/ | 200.3 | P | 551.5 | P | 388.1 | P |
| | | gb = AF012923/gi = 2804680/ug = Mm 35705/len = 7661/STRA = for | | | | | | |
| 18 | 92394_f_at | Cluster Incl AB010266 Mus musculus Creb-rp, Tnx and Cyp21 genes for cAMP response element binding protein-related | 1115.4 | P | 1112.7 | P | 896.2 | P |
| | | protein, tenascin-X and steroid 21-hydroxylase, partial cds and complete sequences/cds = (0,142)/gb = AB010266/ | | | | | | |
| | | gi = 2804287/ug = Mm 4068/len = 515/STRA = for | | | | | | |
| 18 | 92440_at | Cluster Incl U73029 Mus musculus interferon regulatory factor 6 (mirf6) mRNA, complete cds/cds = (141,1544)/gb = U73029/ | 806.3 | P | 1314.3 | P | 794.8 | P |
| | | gi = 1685127/ug = Mm 4179/len = 4046/STRA = rev | | | | | | |
| 18 | 92915_s_at | Cluster Incl X13961 Mouse rearranged homeotic gene Hox-2 4 (IAP Integration)/cds = (0,731)/gb = X13961/gi = 51393/ | 669.7 | P | 693.8 | P | 557.3 | P |
| | | ug = Mm 4822/len = 732/STRA = for | | | | | | |
| 18 | 92922_at | Cluster Incl U17291: Mus musculus transcription factor AP-2 (AP-2) gene, alternative/cds = (36,602)/gb = U17291/ | 1049.3 | P | 1113.2 | P | 1038.3 | P |
| | | gi = 755199/ug = Mm 4845/len = 603/STRA = for | | | | | | |
| 18 | 93454_at | Cluster Incl AF081789 Mus musculus cell surface antigen AA4 (AA4) mRNA, complete cds/cds = (120,2054)/gb = AF081789/ | 381.2 | P | 533.8 | P | 505.4 | P |
| | | gi = 3695112/ug = Mm 681/len = 3070/STRA = for | | | | | | |
| 18 | 93875_at | Cluster Incl M12571: Heat shock protein, 70 kDa 3/cds = (0,1262)/gb = M12571/gi = 194014/ug = Mm 88432/len = 1506/ | 627.1 | P | 797.7 | P | 719.3 | P |
| | | STRA = rev | | | | | | |
| 18 | 96506_at | Cluster Incl D83002: Anaplastic lymphoma kinase/cds = (450,5315)/gb = D83002/gi = 1864006/ug = Mm.2536/len = 5767/ | 258.1 | P | 399.5 | P | 360.9 | P |
| | | STRA = for | | | | | | |
| 18 | 96530_at | Cluster Incl AI503926 vm33f03 x1 Mus musculus cDNA, 3 end/clone = IMAGE-991997/clone_end = 3/gb = AI503926/ | 123.7 | P | 303.3 | P | 158.2 | P |
| | | gi = 4401777/ug = Mm 27091/len = 342/STRA = for | | | | | | |
| 18 | 97722_at | Cluster Incl AA879709 vv97e03 r1 Mus musculus cDNA, 5 end/clone = IMAGE-1230364/clone_end = 5/gb = AA879709/ | 435.9 | P | 665.7 | P | 510.3 | P |
| | | gi = 2988692/ug = Mm.39052/len = 404/STRA = for | | | | | | |
| 18 | 97738_r_at | Cluster Incl AW121931 UI-M-BH2 3-aoj-e-01-0-UI s1 Mus musculus cDNA, 3 end/clone = UI-M-BH2.3-aoj-e-01-0-UI/ | 436 | P | 463.3 | P | 392.3 | P |
| | | clone_end = 3/gb = AW121931/gi = 6097377/ug = Mm 3957/len = 367/STRA = rev | | | | | | |
| 18 | 98308_at | Cluster Incl AJ002522.Mus musculus mRNA for myosin heavy chain 2X, partial/cds = (0,94)/gb = AJ002522/gi = 4582679/ | 198 | P | 2168.5 | P | 1011.6 | P |
| | | ug = Mm 42156/len = 199/STRA = rev | | | | | | |
| 18 | 100357_g_at | Cluster Incl AW121051 UI-M-BH2 3-aof-d-02-0-UI s1 Mus musculus cDNA, 3 end/clone = UI-M-BH2 3-aof-d-02-0-UI/ | 437.5 | P | 536.1 | P | 336.1 | P |
| | | clone_end = 3/gb = AW121051/gi = 6096384/ug = Mm.88761/len = 365/STRA = for | | | | | | |
| 18 | 101291_at | Cluster Incl Z83956 M musculus spermidine synthase pseudogene/cds = (0,894)/gb = Z83956/gi = 1781038/ug = Mm 56916/ | 293.1 | P | 337.1 | P | 268.2 | P |
| | | len = 895/STRA = for | | | | | | |
| 18 | 101795_f_at | Cluster Incl U12566 Defensin related cryptdin, related sequence 12/cds = (0,278)/gb = U12566/gi = 531852/ug = Mm 57135/ | 1077.7 | P | 1026 | P | 936.7 | P |
| | | len = 279/STRA = for | | | | | | |
| 18 | 96375_at | Cluster Incl C80249: C80249 Mus musculus cDNA, 3 end/clone = J0078F09/clone_end = 3/gb = C80249/gi = 2520579/ | 435.6 | P | 684.2 | P | 339.7 | P |
| | | ug = Mm.25065/len = 609/STRA = rev | | | | | | |
| 18 | 102105_f_at | Cluster Incl AI840733: UI-M-AM0-adn-b-04-0-UI s1 Mus musculus cDNA, 3 end/clone = UI-M-AM0-adn-b-04-0-UI/ | 425.9 | P | 410.2 | P | 362.2 | P |
| | | clone_end = 3/gb = AI840733/gi = 5474946/ug = Mm 42506/len = 275/STRA = rev | | | | | | |
| 18 | 96087_at | Cluster Incl L07037 Mannoside acetylglucosaminyl transferase 1/cds = (363,1706)/gb = L07037/gi = 202145/ug = Mm 2672/ | 753.4 | P | 1016.2 | P | 810.4 | P |
| | | len = 2695/STRA = for | | | | | | |
| 18 | 95679_at | Cluster Incl AI835912.UI-M-AI0-aao-a-07-0-UI s1 Mus musculus cDNA, 3 end/clone = UI-M-AI0-aao-a-07-0-UI/ | 395 | P | 684.9 | P | 626.4 | P |
| | | clone_end = 3/gb = AI835912/gi = 5470120/ug = Mm 29609/len = 455/STRA = for | | | | | | |
| 18 | 97546_at | Cluster Incl AF072127 Mus musculus claudin-1 mRNA, complete cds/cds = (155,790)/gb = AF072127/gi = 3335181/ | 222.4 | P | 324.4 | P | 141.5 | P |
| | | ug = Mm 33669/len = 952/STRA = for | | | | | | |
| 18 | 102750_at | Cluster Incl AF070975 Mus musculus X11 gamma protein mRNA, complete cds/cds = (218,1933)/gb = AF070975/ | 431.6 | P | 582.8 | P | 427.6 | P |
| | | gi = 3264793/ug = Mm 12909/len = 2039/STRA = for | | | | | | |
| 18 | 103629_g_at | Cluster Incl D16503.Lymphoid enhancer binding factor 1/cds = (1804,2658)/gb = D16503/gi = 391762/ug = Mm 2029/ | 271.9 | P | 268 | P | 216.3 | P |
| | | len = 2740/STRA = rev | | | | | | |
| 18 | 103900_at | Cluster Incl AW124150 UI-M-BH2 1-apr-f-07-0-UI s1 Mus musculus cDNA, 3 end/clone = UI-M-BH2 1-apr-f-07-0-UI/ | 281.2 | P | 476.1 | P | 287.7 | P |
| | | clone_end = 3/gb = AW124150/gi = 6099680/ug = Mm 2133/len = 254/STRA = for | | | | | | |
| 18 | 95533_at | Cluster Incl AW048037 UI-M-BH1-alv-c-01-0-UI.s1 Mus musculus cDNA, 3 end/clone = UI-M-BH1-alv-c-01-0-UI/ | 302.1 | P | 499.4 | P | 339.8 | P |
| | | clone_end = 3/gb = AW048037/gi = 5908531/ug = Mm.27653/len = 396/STRA = for | | | | | | |
| 18 | 97955_at | Cluster Incl X59513 Mouse 5 end of TRP1 gene for tyrosinase-related protein-1/cds = (185,187)/gb = X59513/gi = 54913/ | 113.6 | P | 214.9 | P | 106 | P |
| | | ug = Mm 30438/len = 297/STRA = for | | | | | | |
| 18 | 98490_at | Cluster Incl AA822412: vw37d10r1 Mus musculus cDNA, 5 end/clone = IMAGE-1246003/clone_end = 5/gb = AA822412/ | 143.4 | P | 181.7 | P | 140.2 | P |
| | | gi = 2892280/ug = Mm 35596/len = 460/STRA = for | | | | | | |
| 18 | 99049_at | Cluster Incl D28492: Caspase 2/cds = (6,1364)/gb = D28492/gi = 3090429/ug = Mm 3921/len = 3395/STRA = for | 774.4 | P | 819.3 | A | 540.1 | P |
| 18 | 102230_at | Cluster Incl AF011567: Hyaluronidase 1/cds = (53,1441)/gb = AF011567/gi = 2961469/ug = Mm 10305/len = 2125/STRA = for | 177 | P | 254.8 | P | 164.1 | P |
| 18 | 95957_at | Cluster Incl C77070 C77070 Mus musculus cDNA, 3 end/clone = J0025F02/clone_end = 3/gb = C77070/gi = 2517400/ | 280.2 | A | 349.5 | P | 255.9 | P |
| | | ug = Mm 24934/len = 592/STRA = rev | | | | | | |
| 18 | 99114_r_at | Cluster Incl AW045905 UI-M-BH1-aku-b-09-0-UI s1 Mus musculus cDNA, 3 end/clone = UI-M-BH1-aku-b-09-0-UI/ | 282.1 | P | 307 | P | 202.8 | P |
| | | clone_end = 3/gb = AW045905/gi = 5906434/ug = Mm 400/len = 464/STRA = for | | | | | | |
| 18 | 102787_at | Cluster Incl AI841654 UI-M-AO0-acg-e-05-0-UI s1 Mus musculus cDNA, 3 end/clone = UI-M-AO0-acg-e-05-0-UI/ | 165.6 | A | 696 | P | 367.9 | P |
| | | clone_end = 3/gb = AI841654/gi = 5475867/ug = Mm 13799/len = 284/STRA = for | | | | | | |
| 18 | 103668_at | Cluster Incl U40375 Suppressor of Ty 6 (S cerevisiae) homolog/cds = (184,5364)/gb = U40375/gi = 1401056/ug = Mm 20755/ | 390.3 | P | 432.5 | P | 403.4 | P |
| | | len = 5923/STRA = for | | | | | | |
| 18 | 104534_at | Cluster Incl AA623874 vq73h02 s1 Mus musculus cDNA, 5 end/clone = IMAGE-1107987/clone_end = 5/gb = AA623874/ | 84 | P | 102.3 | A | 70.8 | P |
| | | gi = 2527750/ug = Mm 2325/len = 318/STRA = for | | | | | | |
| 18 | 99956_at | Cluster Incl Y00864 Mouse c-kit mRNA/cds = (28,2955)/gb = Y00864/gi = 50423/ug = Mm 4394/len = 5098/STRA = for | 193 | P | 237 | A | 167.1 | P |
| 18 | 103281_at | Cluster Incl AF077003: Mus musculus SH3 domain-containing adapter protein mRNA, complete cds/cds = (6,1931)/ | 116.1 | P | 234.7 | A | 133.4 | A |
| | | gb = AF077003/gi = 3550239/ug = Mm 20882/len = 2431/STRA = rev | | | | | | |
| 18 | 92196_f_at | Cluster Incl X83733 M musculus SAP62-AMH gene/cds = (0,1457)/gb = X83733/gi = 633250/ug = Mm 34039/len = 1458/ | 1102.8 | P | 1260.3 | P | 469.3 | A |
| | | STRA = for | | | | | | |
| 18 | 93475_at | Cluster Incl AI845581 UI-M-AQ1-adx-c-02-0-UI s1 Mus musculus cDNA, 3 end/clone = UI-M-AQ1-adx-c-02-0-UI/ | 166.4 | P | 192.5 | P | 173.8 | P |
| | | clone_end = 3/gb = AI845581/gi = 5489487/ug = Mm 7086/len = 136/STRA = rev | | | | | | |
| 18 | 99370_at | Cluster Incl AF095447 Mus musculus natural killer cell receptor NKG2A (Nkg2a) mRNA, complete cds/cds = (0,734)/ | 193.3 | P | 231.2 | P | 213.8 | P |
| | | gb = AF095447/gi = 4115906/ug = Mm 56899/len = 735/STRA = for | | | | | | |
| 18 | 100385_at | Cluster Incl X75832 M musculus Glra1 gene (exon1)/cds = (0,1349)/gb = X75832/gi = 435518/ug = Mm.89320/len = 1350/ | 439.8 | M | 501.6 | P | 494.8 | P |
| | | STRA = rev | | | | | | |
| 18 | 94622_at | Cluster Incl U17252: Mus musculus metabolropic glutamate receptor 8 (mGluR8) mRNA, complete cds/cds = (70,2796)/ | 218.5 | P | 256.6 | M | 161.4 | P |
| | | gb = U17252/gi = 854728/ug = Mm 8001/len = 2830/STRA = for | | | | | | |
| 18 | 99777_s_at | Cluster Incl C76650 C76650 Mus musculus cDNA, 3 end/clone = J0015G08/clone_end = 3/gb = C76650/gi = 2516980/ | 75.8 | A | 105.8 | P | 74.4 | P |
| | | ug = Mm 97081/len = 611/STRA = rev | | | | | | |
| 18 | 95155_at | Cluster Incl AI853875 UI-M-BH0-ajr-g-11-0-UI.s1 Mus musculus cDNA, 3 end/clone = UI-M-BH0-ajr-g-11-0-UI/ | 305.8 | P | 561.9 | A | 301.8 | A |
| | | clone_end = 3/gb = AI853875/gi = 5497781/ug = Mm 29479/len = 416/STRA = for | | | | | | |
| 18 | 101522_at | Cluster Incl L15429 Mus musculus L6 antigen mRNA, complete cds/cds = (91,699)/gb = L15429/gi = 468909/ug = Mm 856/ | 74.9 | P | 129 | P | 103.2 | P |
| | | len = 1077/STRA = for | | | | | | |
| 18 | 97397_at | Cluster Incl AI848344 UI-M-AH1-ago-d-01-0-UI s1 Mus musculus cDNA, 3 end/clone = UI-M-AH1-ago-d-01-0-UI/ | 147.2 | P | 199.6 | P | 141.3 | A |
| | | clone_end = 3/gb = AI848344/gi = 5492250/ug = Mm 29849/len = 404/STRA = for | | | | | | |
| 18 | 100491_at | Cluster Incl AF045692 Solute carrier family 16 (monocarboxylic acid transporters), member 2/cds = (174,1871)/ | 85.9 | P | 114.1 | P | 110.6 | P |
| | | gb = AF045692/gi = 2944355/ug = Mm 5045/len = 4247/STRA = for | | | | | | |
| 18 | 102228_at | Cluster Incl AF036907: Mus musculus linker for activation of T cells (LAT) mRNA, complete cds/cds = (291,1019)/ | 872.8 | P | 1335 | P | 655.8 | A |
| | | gb = AF036907/gi = 2828027/ug = Mm 10280/len = 1260/STRA = for | | | | | | |
| 18 | 103303_at | Cluster Incl AA798246: vy05e06.r1 Mus musculus cDNA, 5 end/clone = IMAGE-1294594/clone_end = 5/gb = AA798246/ | 103.2 | A | 125.6 | P | 73.6 | A |
| | | gi = 2861201/ug = Mm 21099/len = 494/STRA = for | | | | | | |
| 18 | 92303_at | Cluster Incl D31898: Protein tyrosine phosphatase, receptor type, R/cds = (355,2325)/gb = D31898/gi = 695408/ug = Mm.3771/ | 730.9 | A | 969.1 | P | 777.1 | P |
| | | len = 3430/STRA = for | | | | | | |
| 18 | 94197_at | Cluster Incl D89866: Mus musculus mRNA for ceramide glucosyltransferase, complete cds/cds = (185,1369)/gb = D89866/ | 140.1 | P | 238.1 | P | 82.4 | P |
| | | gi = 3219161/ug = Mm.12971/len = 1523/STRA = for | | | | | | |
| 18 | 94733_at | Cluster Incl J03398 P glycoprotein 2/cds = (193,4023)/gb = J03398/gi = 199109/ug = Mm 14172/len = 4084/STRA = for | 343.4 | M | 576.8 | M | 342.5 | P |
| 18 | 98846_f_at | Cluster Incl M83344 Carcinoembryonic antigen 2/cds = (192,1619)/gb = M83344/gi = 200316/ug = Mm 5056/len = 2043/ | 641.8 | P | 1018.4 | P | 410.7 | M |
| | | STRA = for | | | | | | |
| 18 | 101229_at | Cluster Incl C78228: C78228 Mus musculus cDNA, 3 end/clone = J0044F11/clone_end = 3/gb = C78228/gi = 2518558/ | 116.9 | P | 367.1 | P | 84 | A |
| | | ug = Mm 5692/len = 553/STRA = rev | | | | | | |
| 18 | 93516_at | Cluster Incl M25825: T-complex lestis expressed 1/cds = (12,353)/gb = M25825/gi = 201912/ug = Mm.1948/len = 680/ | 100.9 | P | 133.2 | P | 62.3 | A |
| | | STRA = for | | | | | | |
| 18 | 94211_at | Cluster Incl AA959180 ua07g06 r1 Mus musculus cDNA, 5 end/clone = IMAGE-1346074/clone_end = 5/gb = AA959180/ | 434.5 | P | 402.7 | A | 237.9 | A |
| | | gi = 3124373/ug = Mm 22214/len = 347/STRA = for | | | | | | |
| 18 | 97239_at | Cluster Incl AI845106: UI-M-BG0-ahs-a-04-0-UI s1 Mus musculus cDNA, 3 end/clone = UI-M-BG0-ahs-a-04-0-UI/ | 87 | P | 87.6 | P | 44.4 | A |
| | | clone_end = 3/gb = AI845106/gi = 5489012/ug = Mm 27839/len = 141/STRA = for | | | | | | |
| 18 | 103055_r_at | Cluster Incl M12130 RNA polymerase II 1/cds = (0,5798)/gb = M12130/gi = 200793/ug = Mm 16533/len = 5799/STRA = rev | 1219.9 | P | 1110.8 | P | 969.6 | P |
| 18 | 104079_at | Cluster Incl AW124678 UI-M-BH2 1-apw-g-03-0-UI s1 Mus musculus cDNA, 3 end/clone = UI-M-BH2 1-apw-g-03-0-UI/ | 112.2 | P | 271.8 | A | 97.5 | A |
| | | clone_end = 3/gb = AW124678/gi = 6100208/ug = Mm.21735/len = 406/STRA = for | | | | | | |
| 18 | 95011_at | Cluster Incl AI853090 UI-M-BH0-ajf-b-06-0-UI s1 Mus musculus cDNA, 3 end/clone = UI-M-BH0-ajf-b-06-0-UI/ | 161.1 | M | 182.8 | A | 153.7 | P |
| | | clone_end = 3/gb = AI853090/gi = 5496996/ug = Mm 27434/len = 444/STRA = for | | | | | | |
| 18 | 95606_at | Cluster Incl AF093821 Mus musculus RRM RNA binding protein GRY-RBP mRNA, complete cds/cds = (6,1883)/ | 55.6 | A | 69.2 | P | 45.1 | P |
| | | gb = AF093821/gi = 3694985/ug = Mm 27972/len = 2373/STRA = for | | | | | | |
| 18 | 97368_at | Cluster Incl U95145 Mus musculus S-AKAP84 mRNA, complete cds/cds = (197,1840)/gb = U95145/gi = 2072304/ | 1471.4 | P | 1313.2 | P | 1241 | P |
| | | ug = Mm 2969/len = 2965/STRA = for | | | | | | |
| 18 | 97372_at | Cluster Incl AA880432: vw89f04.r1 Mus musculus cDNA, 5 end/clone = IMAGE-1262143/clone_end = 5/gb = AA880432/ | 347.7 | M | 435.4 | M | 369.9 | P |
| | | gi = 2989415/ug = Mm 29701/len = 200/STRA = for | | | | | | |
| 18 | 99523_at | Cluster Incl AA684502 vr02a04 r1 Mus musculus cDNA, 5 end/clone = IMAGE-1110606/clone_end = 5/gb = AA684502/ | 705.8 | M | 1032.1 | P | 614.6 | P |
| | | gi = 2670219/ug = Mm 4206/len = 479/STRA = for | | | | | | |
| 18 | 99931_at | Cluster Incl U37501 Laminin, alpha 5/cds = (0,10907)/gb = U37501/gi = 2599231/ug = Mm 4339/len = 11009/STRA = rev | 137.7 | P | 234.2 | A | 157.9 | A |
| 18 | 100508_at | Cluster Incl AF015769 Mus musculus manic fringe (manic-fringe) mRNA, complete cds/cds = (0,965)/gb = AF015769/ | 314 | P | 440.3 | P | 323. 5 | P |
| | | gi = 2454571/ug = Mm 517/len = 966/STRA = for | | | | | | |
| 18 | 101932_at | Cluster Incl D83484 Mouse mRNA for protein tyrosine phosphatase epsilon, complete cds/cds = (660,2759)/gb = D83484/ | 242.3 | P | 418.2 | P | 129.5 | A |
| | | gi = 1199932/ug = Mm 945/len = 2827/STRA = for | | | | | | |
| 18 | 92754_at | Cluster Incl D49920 Mouse mRNA for ferredoxin-NADP reductase, complete cds/cds = (82,1566)/gb = D49920/gi = 1088468/ | 318 | M | 310.3 | P | 316.8 | P |
| | | ug = Mm 4719/len = 1866/STRA = rev | | | | | | |
| 18 | 92936_at | Cluster Incl X14943 Contactin 1/cds = (249,3311)/gb = X14943/gi = 50937/ug = Mm 4911/len = 3843/STRA = for | 73 | M | 297.9 | P | 52.5 | A |
| 18 | 98312_at | Cluster Incl AB016768 Mus musculus mRNA for thrombospondin type 1 domain, complete cds/cds = (482,1279)/ | 106.3 | P | 260.1 | P | 121.4 | A |
| | | gb = AB016768/gi = 4519540/ug = Mm 42202/len = 1834/STRA = for | | | | | | |
| 18 | 98385_at | Cluster Incl D31842: Mouse mRNA for putative protein tyrosine phosphatase (PTP36), complete cds/cds = (133,3702)/ | 181.7 | P | 393.7 | P | 240.7 | M |
| | | gb = D31842/gi = 507330/ug = Mm 4498/len = 3957/STRA = rev | | | | | | |
| 18 | 101148_at | Cluster Incl D28577 Protein kinase C, lamda/cds = (36,1796)/gb = D28577/gi = 3452685/ug = Mm 4801/len = 1966/STRA = for | 152.4 | M | 243.6 | A | 210 | P |
| 18 | 97894_at | Cluster Incl AF109905 Mus musculus major histocompatibility locus class III regions Hsc70t gene, partial cds, smRNP, | 359.2 | P | 419.7 | P | 412.4 | A |
| | | G7A, NG23, Muts homolog, CLCP, NG24, NG25, and NG26 genes, complete cds, and unknown genes/cds = (0,3791)/ | | | | | | |
| | | gb = AF109905/gi = 3986751/ug = Mm 2/STRA = for | | | | | | |
| 18 | 94463_at | Cluster Incl X78874: Chloride channel 3/cds = (375,2657)/gb = X78874/gi = 854275/ug = Mm.28842/len = 2758/STRA = for | 82.1 | A | 529.9 | A | 95.2 | P |
| 18 | 102769_f_at | Cluster Incl AB016248 Mus musculus mRNA for sterol-C5-desaturase, complete cds/cds = (170,1069)/gb = AB016248/ | 47 | A | 83.1 | A | 38.4 | P |
| | | gi = 3721883/ug = Mm 13081/len = 1721/STRA = for | | | | | | |
| 18 | 103635_at | Cluster Incl AI648925 uk32h09 x1 Mus musculus cDNA, 3 end/clone = IMAGE-1970753/clone_end = 3/gb = AI648925/ | 41.8 | A | 155.6 | A | 50.6 | P |
| | | gi = 4729759/ug = Mm 20320/len = 594/STRA = for | | | | | | |
| 18 | 104326_at | Cluster Incl AI838951 UI-M-AO0-ace-e-06-0-UI s1 Mus musculus cDNA, 3 end/clone = UI-M-AO0-ace-e-06-0-UI/ | 326.5 | P | 519.7 | A | 255.6 | A |
| | | clone_end = 3/gb = AI838951/gi = 5473164/ug = Mm 22456/len = 451/STRA = for | | | | | | |
| 18 | 96817_at | Cluster Incl AI850550 UI-M-BG1-aij-d-11-0-UI s1 Mus musculus cDNA, 3 end/clone = UI-M-BG1-aij-d-11-0-UI/ | 280.3 | P | 284.4 | P | 207.8 | A |
| | | clone_end = 3/gb = AI850550/gi = 5494456/ug = Mm 29333/len = 282/STRA = for | | | | | | |
| 18 | 100906_at | Cluster Incl M68903 Integrin beta 7/cds = (39,2456)/gb = M68903/gi = 349598/ug = Mm 58/len = 2636/STRA = rev | 399.3 | A | 462.6 | P | 374.6 | M |
| 18 | 103756_at | Cluster Incl AA832886 uc91h02 r1 Mus musculus cDNA, 5 end/clone = IMAGE-1433043/clone_end = 5/gb = AA832886/ | 237.2 | A | 209.6 | P | 183.9 | A |
| | | gi = 2906614/ug = Mm.24569/len = 395/STRA = rev | | | | | | |
| 18 | 93391_at | Cluster Incl AF032995: Crystallin, gamma S/cds = (51,587)/gb = AF032995/gi = 2642336/ug = Mm 6253/len = 702/STRA = for | 60.6 | M | 104.9 | M | 75.3 | P |
| 18 | 93902_at | Cluster Incl AJ250669: Mus musculus gab1 gene for GRB2-associated binding protein 1/cds = (23,2110)/gb = AJ250669/ | 168.9 | P | 221.8 | A | 143.5 | A |
| | | gi = 6165414/ug = Mm.89347/len = 2620/STRA = for | | | | | | |
| 18 | 94111_r_at | Cluster Incl D10627: Mus musculus mRNA for zinc finger protein, partial cds/cds = (0,1040)/gb = D10627/gi = 220636/ | 73.7 | A | 152 | P | 65.3 | A |
| | | ug = Mm 10509/len = 1041/STRA = for | | | | | | |
| 18 | 96502_at | Cluster Incl U75646 Phosphate regulating neutral endopeptidases on the X Chromosome/cds = (111,2360)/gb = U75646/ | 123.9 | P | 140.5 | P | 108 | A |
| | | gi = 1843530/ug = Mm.2529/len = 2636/STRA = for | | | | | | |
| 18 | 97107_at | Cluster Incl AA162144 mr19b05 r1 Mus musculus cDNA, 5 end/clone = IMAGE-597873/clone_end = 5/gb = AA162144/ | 33.8 | P | 90.5 | A | 49.2 | P |
| | | gi = 1737885/ug = Mm.3261/len = 300/STRA = rev | | | | | | |
| 18 | 99894_at | Cluster Incl AF006201: Mus musculus prostaglandin F2 alpha receptor regulatory protein mRNA, partial cds/cds = (0,198)/ | 267.8 | A | 393.1 | P | 200.7 | A |
| | | gb = AF006201/gi = 2653775/ug = Mm 7999/len = 271/STRA = for | | | | | | |
| 18 | 100701_r_at | Cluster Incl AB000490 Mouse mRNA for ELP3, complete cds/cds = (245,1636)/gb = AB000490/gi = 1805352/ug = Mm 1416/ | 189.4 | P | 167.2 | P | 164.7 | P |
| | | len = 2981/STRA = for | | | | | | |
| 18 | 101705_at | Cluster Incl X12809: Mouse period repetitive mRNA (lambda Sp34)/cds = (30,212)/gb = X12809/gi = 53648/ug = Mm 41987/ | 318 | P | 285.2 | P | 279.4 | A |
| | | len = 522/STRA = for | | | | | | |
| 18 | 93778_at | Cluster Incl AA717815 vt98f07 r1 Mus musculus cDNA, 5 end/clone = IMAGE-1179205/clone_end = 5/gb = AA717815/ | 665.5 | A | 746.1 | P | 624.4 | P |
| | | gi = 2730089/ug = Mm 21239/len = 424/STRA = for | | | | | | |
| 18 | 104608_at | Cluster Incl AI854293: UI-M-BH0-ajs-e-11-0-UI s1 Mus musculus cDNA, 3 end/clone = UI-M-BH0-ajs-e-11-0-UI/ | 78.7 | P | 94.6 | M | 85.6 | A |
| | | clone_end = 3/gb = AI854293/gi = 5498199/ug = Mm 24143/len = 511/STRA = for | | | | | | |
| 18 | 94349_at | Cluster Incl AA794923: vr48g02.s1 Mus musculus cDNA, 5 end/clone = IMAGE-1123922/clone_end = 5/gb = AA794923/ | 71.9 | A | 204.4 | P | 127.1 | P |
| | | gi = 2857878/ug = Mm 25189/len = 537/STRA = for | | | | | | |
| 18 | 97980_at | Cluster Incl L38423: Lymphotoxin B receptor/cds = (185,1432)/gb = L38423/gi = 600222/ug = Mm.3122/len = 1614/STRA = for | 293.5 | A | 425.7 | A | 312.6 | P |
| 18 | 101857_at | Cluster Incl AB006036 Serine/arginine-rich protein specific kinase 2/cds = (0,2045)/gb = AB006036/gi = 2723281/ | 81 | A | 109.9 | P | 66.9 | A |
| | | ug = Mm B709/len = 2300/STRA = for | | | | | | |
| 18 | 103473_at | Cluster Incl AA623401 vn32c09.r1 Mus musculus cDNA, 5 end/clone = IMAGE-1022896/clone_end = 5/gb = AA623401/ | 313.3 | A | 535.8 | P | 330.9 | A |
| | | gi = 2527277/ug = Mm 21876/len = 345/STRA = for | | | | | | |
| 18 | 104413_at | Cluster Incl AI834976 UI-M-AM1-afw-d-12-0-UI s1 Mus musculus cDNA, 3 end/clone = UI-M-AM1-afw-d-12-0-UI/ | 109.3 | P | 107.8 | A | 72 | A |
| | | clone_end = 3/gb = AI834976/gi = 5469189/ug = Mm.28885/len = 434/STRA = for | | | | | | |
| 18 | 93899_at | Cluster Incl AJ000654 Mouse p70/p85 S6 kinase gene, 5 flanking region and the first exon/cds = (63,203)/gb = AJ000654/ | 90.9 | A | 175.2 | P | 137.8 | A |
| | | gi = 2330619/ug = Mm 89315/len = 204/STRA = for | | | | | | |
| 18 | 100326_f_at | Cluster Incl U12889 Killer cell lectin-like receptor, subfamily A, member B/cds = (196,996)/gb = U12889/gi = 602407/ | 61.7 | P | 162.2 | P | 45.8 | P |
| | | ug = Mm.87782/len = 1229/STRA = for | | | | | | |
| 18 | 96961_at | Cluster Incl AI503821: vm20c01.x1 Mus musculus cDNA, 3 end/clone = IMAGE-990720/clone_end = 3/gb = AI503821/ | 139 | A | 206 | P | 143.9 | P |
| | | gi = 4401672/ug = Mm 69094/len = 470/STRA = rev | | | | | | |
| 18 | 102131_f_at | Cluster Incl AU014874 AU014874 Mus musculus cDNA, 3 end/clone = J0703H02/clone_end = 3/gb = AU014874/ | 497.8 | M | 508.2 | M | 443.3 | P |
| | | gi = 3369665/ug = Mm 43527/len = 294/STRA = rev | | | | | | |
| 19 | 92616_at | Cluster Incl D10576 Ubiquitin-activating enzyme E1, Chr X/cds = (91,3267)/gb = D10576/gi = 220628/ug = Mm 1104/ | 1567.6 | P | 1403.1 | P | 1496.7 | P |
| | | len = 3458/STRA = for | | | | | | |
| 19 | 92793_at | Cluster Incl X57796 Tumor necrosis factor receptor superfamily, member 1a/cds = (272,1636)/gb = X57796/gi = 54848/ | 635.5 | P | 841.3 | P | 734.3 | P |
| | | ug = Mm 1258/len = 2179/STRA = for | | | | | | |
| 19 | 93047_at | Cluster Incl AI849144 UI-M-AJ1-agy-c-07-0-UI.s1 Mus musculus cDNA, 3 end/clone = UI-M-AJ1-agy-c-07-0-UI/ | 991.4 | P | 941.5 | P | 874.3 | P |
| | | clone_end = 3/gb = AI849144/gi = 5493050/ug = Mm.1521/len = 439/STRA = for | | | | | | |
| 19 | 93083_at | Cluster Incl D63423: Mouse mRNA for annexin V, complete cds/cds = (81,1040)/gb = D63423/gi = 939830/ug = Mm 1620/ | 1649.8 | P | 1252.8 | P | 1331 | P |
| | | len = 1415/STRA = for | | | | | | |
| 19 | 93255_at | Cluster lncl X80937 Ral-interacting protein 1/cds = (202,2148)/gb = X80937/gi = 984748/ug = Mm 17009/len = 3661/ | 1461.3 | P | 1846 | P | 1713.1 | P |
| | | STRA = rev | | | | | | |
| 19 | 93268_at | Cluster Incl AI852001 UI-M-BH0-aiq-f-01-0-UI s1 Mus musculus cDNA, 3 end/clone = UI-M-BH0-aiq-f-01-0-UI/ | 858.1 | P | 843.6 | P | 903.3 | P |
| | | clone_end = 3/gb = AI852001/gi = 5495907/ug = Mm 17362/len = 431/STRA = for | | | | | | |
| 19 | 93269_at | Cluster Incl AI848952 UI-M-AJ1-ahd-a-06-0-UI s1 Mus musculus cDNA, 3 end/clone = UI-M-AJ1-ahd-a-06-0-UI/ | 1034.9 | P | 1287.1 | P | 1851.1 | P |
| | | clone_end = 3/gb = AI848952/gi = 5492858/ug = Mm 17362/len = 482/STRA = for | | | | | | |
| 19 | 93315_at | Cluster Incl AI852636 UI-M-BH0-aiu-g-03-0-Ul.s1 Mus musculus cDNA, 3 end/clone = UI-M-BH0-aiu-g-03-0-UI/ | 801.9 | P | 903.8 | P | 937.3 | P |
| | | clone_end = 3/gb = AI852636/gi = 5496542/ug = Mm 18494/len = 365/STRA = for | | | | | | |
| 19 | 93323_at | Cluster Incl AB031292 Mus musculus Plp2 mRNA for proteolipid protein 2, complete cds/cds = (58,516)/gb = AB031292/ | 3961.1 | P | 4061.2 | P | 4110.8 | P |
| | | gi = 5771450/ug = Mm 18585/len = 603/STRA = for | | | | | | |
| 19 | 93336_at | Cluster Incl AW121539: UI-M-BH2 2-aop-e-01-0-UI s1 Mus musculus cDNA, 3 end/clone = UI-M-BH2 2-aop-e-01-0-UI/ | 1488.3 | P | 1449.3 | P | 1730.4 | P |
| | | clone_end = 3/gb = AW121539/gi = 6096872/ug = Mm 18688/len = 514/STRA = for | | | | | | |
| 19 | 93753_at | Cluster Incl AI852632: UI-M-BH0-aiu-f-10-0-UI.s1 Mus musculus cDNA, 3 end/clone = UI-M-BH0-aiu-f-10-0-UI/ | 728.3 | P | 806.8 | P | 740.7 | P |
| | | clone_end = 3/gb = AI852632/gi = 5496538/ug = Mm 21119/len = 346/STRA = for | | | | | | |
| 19 | 93809_at | Cluster Incl U41736 Ancient ubiquitous protein/cds = (60,1292)/gb = U41736/gi = 1517821/ug = Mm.2146/len = 1499/ | 938.4 | P | 811.4 | P | 962.5 | P |
| | | STRA = for | | | | | | |
| 19 | 93843_at | Cluster Incl AI845580 UI-M-AQ1-adx-c-01-0-UI s1 Mus musculus cDNA, 3 end/clone = UI-M-AQ1-adx-c-01-0-UI/ | 1022.5 | P | 934.8 | P | 981.7 | P |
| | | clone_end = 3/gb = AI845580/gi = 5489486/ug = Mm.21623/len = 245/STRA = for | | | | | | |
| 19 | 94297_at | Cluster Incl U16959 FK506 binding protein 5, 51 kDa/cds = (219,1589)/gb = U16959/gi = 915279/ug = Mm 22595/len = 2285/ | 818.1 | P | 534.8 | P | 721.2 | P |
| | | STRA = for | | | | | | |
| 19 | 94805_f_at | Cluster Incl M33988 Mouse histone H2A 1 gene, complete cds/cds = (45,437)/gb = M33988/gi = 193730/ug = Mm 22676/ | 3584.3 | P | 4048.9 | P | 4488.6 | P |
| | | len = 506/STRA = for | | | | | | |
| 19 | 94813_at | Cluster Incl X65128 Growth arrest specific 1/cds = (424,1578)/gb = X65128/gi = 51045/ug = Mm 22701/len = 2961/STRA = for | 2336 | P | 1833.9 | P | 2065.1 | P |
| 19 | 94850_at | Cluster Incl AJ238894 Mus musculus mRNA for mitochondrial acyl-CoA thioesterase, clone 2/cds = (97,1416)/ | 586.9 | P | 609.4 | P | 745.9 | P |
| | | gb = AJ238894/gi = 5102775/ug = Mm 23368/len = 1557/STRA = for | | | | | | |
| 19 | 94868_at | Cluster Incl AW049812 UI-M-BH1-anm-h-04-0-UI s1 Mus musculus cDNA, 3 end/clone = UI-M-BH1-anm-h-04-0-UI/ | 1178.9 | P | 1204.2 | P | 1504.6 | P |
| | | clone_end = 3/gb = AW049812/gi = 5910341/ug = Mm 23750/len = 571/STRA = for | | | | | | |
| 19 | 94881_at | Cluster Incl AW048937 UI-M-BH1-amo-d-08-0-UI s1 Mus musculus cDNA, 3 end/clone = UI-M-BH1-amo-d-08-0-UI/ | 1483.5 | P | 1421.9 | P | 1468.4 | P |
| | | clone_end = 3/gb = AW048937/gi = 5909466/ug = Mm 2387/len = 355/STRA = for | | | | | | |
| 19 | 95446_at | Cluster Incl AW120502 UI-M-BH1-ami-f-05-0-UI s2 Mus musculus cDNA, 3 end/clone = UI-M-BH1-ami-f-05-0-UI/ | 1246.1 | P | 1081.5 | P | 1148.4 | P |
| | | clone_end = 3/gb = AW120502/gi = 6095835/ug = Mm 24600/len = 199/STRA = for | | | | | | |
| 19 | 95520_at | Cluster Incl AI843845 UI-M-AK1-aeu-a-10-0-UI s1 Mus musculus cDNA, 3 end/clone = UI-M-AK1-aeu-a-10-0-UI/ | 281.8 | P | 231.6 | P | 383.1 | P |
| | | clone_end = 3/gb = AI843845/gi = 5478058/ug = Mm 25320/len = 404/STRA = for | | | | | | |
| 19 | 96056_at | Cluster Incl X80638 M. musculus rhoC mRNA/cds = (40,621)/gb = X80638/gi = 1279555/ug = Mm.262/len = 1036/STRA = for | 858.3 | P | 767.6 | P | 804.5 | P |
| 19 | 96060_at | Cluster Incl U25844 Serine protease inhibitor 3/cds = (67,1203)/gb = U25844/gi = 818902/ug = Mm 2623/len = 1317/ | 1089.9 | P | 1068.5 | P | 1579 | P |
| | | STRA = for | | | | | | |
| 19 | 96102_i_at | Cluster Incl X92411 RAD23b homolog (S cerevisiae)/cds = (0,1250)/gb = X92411/gi = 1044898/ug = Mm 26879/len = 1251/ | 592.7 | P | 631.5 | P | 619.8 | P |
| | | STRA = for | | | | | | |
| 19 | 96113_at | Cluster Incl AI846519 UI-M-AN1-aff-e-06-0-UI s1 Mus musculus cDNA, 3 end/clone = UI-M-AN1-aff-e-06-0-UI/ | 931.9 | P | 737.1 | P | 1099.8 | P |
| | | clone_end = 3/gb = AI846519/gi = 5490425/ug = Mm 26956/len = 234/STRA = for | | | | | | |
| 19 | 96117_r_at | Cluster Incl AI843732: UI-M-AK1-aer-g-09-0-UI s1 Mus musculus cDNA, 3 end/clone = UI-M-AK1-aer-g-09-0-UI/ | 602.3 | P | 551.7 | P | 781.5 | P |
| | | clone_end = 3/gb = AI843732/gi = 5477945/ug = Mm 26991/len = 369/STRA = for | | | | | | |
| 19 | 96709_at | Cluster Incl AI839839: UI-M-AP0-abf-a-09-0-UI s2 Mus musculus cDNA, 3 end/clone = UI-M-AP0-abf-a-09-0-UI/ | 886 | P | 699.7 | P | 953 | P |
| | | clone_end = 3/gb = AI839839/gi = 5474052/ug = Mm 27608/len = 281/STRA = for | | | | | | |
| 19 | 97885_at | Clusler Incl AB031386 Mus musculus mRNA for Clast1, complete cds/cds = (196,987)/gb = AB031386/gi = 5804793/ | 1312.4 | P | 1302.7 | P | 1567.4 | P |
| | | ug = Mm 28385/len = 1207/STRA = for | | | | | | |
| 19 | 98884_r_at | Cluster Incl AI837311: UI-M-AK0-adf-b-03-0-UI.s1 Mus musculus cDNA, 3 end/clone = UI-M-AK0-adf-b-03-0-UI/ | 961.7 | P | 1001.8 | P | 865.1 | P |
| | | clone_end = 3/gb = AI837311/gi = 5471524/ug = Mm.28527/len = 420/STRA = for | | | | | | |
| 19 | 94465_g_at | Cluster Incl AF029347: Mus musculus chloride channel protein 3 (CLCN3) mRNA, complete cds/cds = (0,2456)/ | 188.5 | P | 196.6 | P | 232.8 | P |
| | | gb = AF029347/gi = 2599549/ug = Mm.28842/len = 2457/STRA = for | | | | | | |
| 19 | 94473_at | Cluster Incl AW047951: UI-M-BH1-aln-f-04-0-UI s1 Mus musculus cDNA, 3 end/clone = UI-M-BH1-aln-f-04-0-UI/ | 639.8 | P | 701.5 | P | 809.7 | P |
| | | clone_end = 3/gb = AW047951/gi = 5908480/ug = Mm 28853/len = 408/STRA = for | | | | | | |
| 19 | 94484_at | Cluster Incl AW227778: up14b01.y1 Mus musculus cDNA, 5 end/clone = IMAGE-2654281/clone_end = 5/gb = AW227778/ | 637.6 | P | 695.5 | P | 648.4 | P |
| | | gi = 6557074/ug = Mm 28882/len = 460/STRA = for | | | | | | |
| 19 | 94485_at | Cluster Incl AI840013 UI-M-AH0-acu-e-04-0-UI s1 Mus musculus cDNA, 3 end/clone = UI-M-AH0-acu-e-04-0-UI/ | 829 | P | 1025.5 | P | 1064.5 | P |
| | | clone_end = 3/gb = AI840013/gi = 5474226/ug = Mm 28883/len = 356/STRA = for | | | | | | |
| 19 | 94530_at | Cluster Incl AI840376 UI-M-AH0-acv-d-02-0-UI s1 Mus musculus cDNA, 3 end/clone = UI-M-AH0-acv-d-02-0-UI/ | 238.5 | P | 289.1 | P | 211 | P |
| | | clone_end = 3/gb = AI840376/gi = 5474589/ug = Mm 29041/len = 448/STRA = for | | | | | | |
| 19 | 95082_at | Cluster Incl AI842277 UI-M-AM1-afz-e-02-0-UI.s1 Mus musculus cDNA, 3 end/clone = UI-M-AM1-afz-e-02-0-UI/ | 689.1 | P | 837.5 | P | 1257.6 | P |
| | | clone_end = 3/gb = AI842277/gi = 5476530/ug = Mm.29254/len = 475/STRA = for | | | | | | |
| 19 | 95129_at | Cluster Incl AW121057 UI-M-BH2 3-aof-d-08-0-UI s1 Mus musculus cDNA, 3 end/clone = UI-M-BH2 3-aof-d-08-0-UI/ | 865.7 | P | 1198.5 | P | 1046.6 | P |
| | | clone_end = 3/gb = AW121057/gi = 6096390/ug = Mm 29412/len = 220/STRA = rev | | | | | | |
| 19 | 95133_at | Cluster Incl U38940 Mus musculus asparagine synthetase mRNA, complete cds/cds = (98,1783)/gb = U38940/gi = 1147604/ | 1261 | P | 1640.4 | P | 1359.8 | P |
| | | ug = Mm.2942/len = 1928/STRA = for | | | | | | |
| 19 | 95721_at | Cluster Incl AW120722 UI-M-BH2.3-any-a-09-0-UI s1 Mus musculus cDNA, 3 end/clone = UI-M-BH2 3-any-a-09-0-UI/ | 1225.6 | P | 1150.6 | P | 1604.1 | P |
| | | clone_end = 3/gb = AW120722/gi = 6096055/ug = Mm 29725/len = 406/STRA = for | | | | | | |
| 19 | 96278_at | Cluster Incl AI846553 UI-M-AN1-aff-h-09-0-UI s1 Mus musculus cDNA, 3 end/clone = UI-M-AN1-aff-h-09-0-UI/ | 297.7 | P | 383.2 | P | 307 | P |
| | | clone_end = 3/gb = AI846553/gi = 5490459/ug = Mm 29865/len = 300/STRA = for | | | | | | |
| 19 | 96359_at | Cluster Incl AI836554 UI-M-AP0-abe-e-12-0-UI s1 Mus musculus cDNA, 3 end/clone = UI-M-AP0-abe-e-12-0-UI/ | 1402 | P | 1580.5 | P | 1406.5 | P |
| | | clone_end = 3/gb = AI836554/gi = 5470767/ug = Mm 30012/len = 514/STRA = for | | | | | | |
| 19 | 96919_at | Cluster Incl M64298 ATPase-like vacuolar proton channel/cds = (147,614)/gb = M64298/gi = 199901/ug = Mm.30155/ | 3143.5 | P | 3307.6 | P | 3426 | P |
| | | len = 1153/STRA = for | | | | | | |
| 19 | 96935_at | Cluster Incl AW011791 ul91f11 x1 Mus musculus cDNA, 3 end/clone = IMAGE-2159469/clone_end = 3/gb = AW011791/ | 833.9 | P | 881.8 | P | 728.3 | P |
| | | gi = 5860569/ug = Mm 30181/len = 290/STRA = for | | | | | | |
| 19 | 96953_at | Cluster Incl AW120786 UI-M-BH2 3-any-g-10-0-UI s1 Mus musculus cDNA, 3 end/clone = UI-M-BH2 3-any-g-10-0-UI/ | 939.1 | P | 1048.3 | P | 890.8 | P |
| | | clone_end = 3/gb = AW120786/gi = 6096119/ug = Mm 30211/len = 401/STRA = for | | | | | | |
| 19 | 97487_at | Cluster Incl X70296 Serine protease Inhibitor 4/cds = (148,1341)/gb = X70296/gi = 551064/ug = Mm 3093/len = 2024/ | 194.6 | P | 247.9 | P | 267.6 | P |
| | | STRA = for | | | | | | |
| 19 | 97498_at | Cluster Incl U41739: Mus musculus RBP associated molecule RAM14-1 mRNA, complete cds/cds = (82,924)/gb = U41739/ | 3645.3 | P | 3700.7 | P | 4198.9 | P |
| | | gi = 2894671/ug = Mm.3126/len = 2270/STRA = for | | | | | | |
| 19 | 97500_g_at | Cluster Incl AI839950 UI-M-AH0-acs-a-10-0-UI s1 Mus musculus cDNA, 3 end/clone = UI-M-AH0-acs-a-10-0-UI/ | 1870.3 | P | 2441.5 | P | 2637.9 | P |
| | | clone_end = 3/gb = AI839950/gi = 5474163/ug = Mm 3126/len = 361/STRA = for | | | | | | |
| 19 | 97551_at | Cluster Incl AI846182 UI-M-AM1-agb-c-09-0-UI.s1 Mus musculus cDNA, 3 end/clone = UI-M-AM1-agb-c-09-0-UI/ | 476.8 | P | 561.9 | P | 711.6 | P |
| | | clone_end = 3/gb = AI846182/gi = 5490088/ug = Mm 33797/len = 312/STRA = for | | | | | | |
| 19 | 98051_at | Cluster Incl AI852808 UI-M-BH0-aly-g-09-0-UI.s1 Mus musculus cDNA, 3 end/clone = UI-M-BH0-aly-g-09-0-UI/ | 702.8 | P | 601.7 | P | 733.2 | P |
| | | clone_end = 3/gb = AI852808/gi = 5496714/ug = Mm 34315/len = 359/STRA = for | | | | | | |
| 19 | 98057_at | Cluster Incl AW121162 UI-M-BH2 3-aoh-g-10-0-UI s1 Mus musculus cDNA, 3 end/clone = UI-M-BH2 3-aoh-g-10-0-UI/ | 138.2 | P | 98.3 | P | 172.1 | P |
| | | clone_end = 3/gb = AW121162/gi = 6096495/ug = Mm 34356/len = 429/STRA = for | | | | | | |
| 19 | 98081_at | Cluster Incl AI853173: UI-M-BH0-ajh-b-07-0-UI s1 Mus musculus cDNA, 3 end/clone = UI-M-BH0-ajh-b-07-0-UI/ | 393.8 | P | 389.7 | P | 389.2 | P |
| | | clone_end = 3/gb = AI853173/gi = 5497079/ug = Mm 34570/len = 366/STRA = for | | | | | | |
| 19 | 98560_at | Cluster Incl AI849152: UI-M-AJ1-agy-d-04-0-UI s1 Mus musculus cDNA, 3 end/clone = UI-M-AJ1-agy-d-04-0-UI/ | 611.1 | P | 733.2 | P | 679.8 | P |
| | | clone_end = 3/gb = AI849152/gi = 5493058/ug = Mm 36879/len = 435/STRA = for | | | | | | |
| 19 | 98580_at | Cluster Incl D28117 Mouse mRNA for magnesium dependent protein phosphatase (protein phosphatase 2C) alpha, | 671.2 | P | 655.5 | P | 656.2 | P |
| | | complete cds/cds = (117,1265)/gb = D28117/gi = 532678/ug = Mm 3766/len = 1387/STRA = for | | | | | | |
| 19 | 99099_at | Cluster Incl U08378 Mus musculus acute phase response factor (APRF) mRNA, complete cds/cds = (146,2455)/ | 1162.7 | P | 1294.7 | P | 964.3 | P |
| | | gb = U08378/gi = 473889/ug = Mm 3948/len = 2924/STRA = for | | | | | | |
| 19 | 99606_at | Cluster Incl AB005132: LIM motif-containing protein kinase 2/cds = (355,2208)/gb = AB005132/gi = 2804552/ug = Mm 42927/ | 575.1 | P | 726.6 | P | 694.6 | P |
| | | len = 3688/STRA = for | | | | | | |
| 19 | 99622_at | Cluster Incl U20344 Gut enriched Kruppel-like factor/cds = (310,1761)/gb = U20344/gi = 912487/ug = Mm 4325/len = 2756/ | 3055.4 | P | 3433.1 | P | 3217.7 | P |
| | | STRA = for | | | | | | |
| 19 | 99635_at | Cluster Incl AI845183 UI-M-BG0-aht-a-12-0-UI s1 Mus musculus cDNA, 3 end/clone = UI-M-BG0-aht-a-12-0-UI/ | 925.9 | P | 914.6 | P | 992.8 | P |
| | | clone_end = 3/gb = AI845183/gi = 5489089/ug = Mm 43482/len = 412/STRA = for | | | | | | |
| 19 | 99636_at | Cluster Incl AI848033: UI-M-AH1-agp-f-05-0-UI s1 Mus musculus cDNA, 3 end/clone = UI-M-AH1-agp-f-05-0-UI/ | 467.8 | P | 435.7 | P | 508.6 | P |
| | | clone_end = 3/gb = AI848033/gi = 5491939/ug = Mm 43487/len = 257/STRA = for | | | | | | |
| 19 | 99638_at | Cluster Incl D17546 Procoliagen, type XV/cds = (0,3422)/gb = D17546/gi = 2160436/ug = Mm 4352/len = 4437/STRA = for | 675.1 | P | 759.4 | P | 714.7 | P |
| 19 | 100095_at | Cluster Incl U37799 Scavenger receptor class B1/cds = (50,1579)/gb = U37799/gi = 1167551/ug = Mm 4603/len = 1785/ | 695.8 | P | 773.1 | P | 823.8 | P |
| | | STRA = for | | | | | | |
| 19 | 100134_at | Cluster Incl X77952 Endoglin/cds = (363,2324)/gb = X77952/gi = 458473/ug = Mm 4851/len = 2902/STRA = for | 689.2 | P | 554.7 | P | 949 | P |
| 19 | 100530_at | Cluster Incl L07924 Mus musculus guanine nucleotide dissociation stimulator for a ras-related GTPase mRNA, complete | 1050.7 | P | 1004.4 | P | 1160.4 | P |
| | | cds/cds = (212,2770)/gb = L07924/gi = 193572/ug = Mm 5236/len = 3573/STRA = for | | | | | | |
| 19 | 100573_f_at | Cluster Incl M14220 Mouse neuroleukin mRNA, complete cds/cds = (52,1728)/gb = M14220/gi = 200064/ug = Mm 589/ | 683.6 | P | 603.1 | P | 720.4 | P |
| | | len = 1985/STRA = for | | | | | | |
| 19 | 100593_at | Cluster Incl L47600 Mus musculus cardiac troponin T isoform A3b mRNA, complete cds/cds = (56,922)/gb = L47600/ | 164.4 | P | 203.5 | P | 209.6 | P |
| | | gi = 1161079/ug = Mm 632/len = 1111/STRA = for | | | | | | |
| 19 | 100599_at | Cluster Incl M94087: Activating transcription factor 4/cds = (270,1310)/gb = M94087/gi = 293723/ug = Mm 641/len = 1391/ | 1283.3 | P | 1405.6 | P | 1392.1 | P |
| | | STRA = for | | | | | | |
| 19 | 100636_at | Cluster Incl U28656: Eukaryotic translation Initiation factor 4E binding protein 1/cds = (50,403)/gb = U28656/gi = 881557/ | 532.9 | P | 565.9 | P | 612.4 | P |
| | | ug = Mm.6700/len = 793/STRA = for | | | | | | |
| 19 | 101011_at | Cluster Incl L25913: Chaperonin subunit 4 (delta)/cds = (0,1619)/gb = L25913/gi = 460316/ug = Mm.6821/len = 1847/ | 1755.2 | P | 1780.3 | P | 1885.1 | P |
| | | STRA = for | | | | | | |
| 19 | 101089_at | Cluster Incl AF002283 Mus musculus alpha-actinin-2 associated LIM protein mRNA, alternatively spliced product, complete | 868.5 | P | 735.3 | P | 917.7 | P |
| | | cds/cds = (119,1069)/gb = AF002283/gi = 3138925/ug = Mm 7342/len = 1485/STRA = for | | | | | | |
| 19 | 101591_at | Cluster Incl AI852589 UI-M-BH0-alu-b-07-0-UI s1 Mus musculus cDNA, 3 end/clone = UI-M-BH0-alu-b-07-0-UI/ | 449.8 | P | 471.5 | P | 458.1 | P |
| | | clone_end = 3/gb = AI852589/gi = 5496495/ug = Mm.91334/len = 235/STRA = for | | | | | | |
| 19 | 101975_at | Cluster Incl Z12171 DIk1-like homolog (Drosophila)/cds = (0,1157)/gb = Z12171/gi = 50716/ug = Mm 95633/len = 1158/ | 4661.6 | P | 4232.7 | P | 5260.4 | P |
| | | STRA = for | | | | | | |
| 19 | 102072_g_at | Cluster Incl AI851565 UI-M-BH0-aki-f-01-0-UI.s1 Mus musculus cDNA, 3 end/clone = UI-M-BH0-aki-f-01-0-UI/clone_end = 3/ | 586.3 | P | 558.4 | P | 554.4 | P |
| | | gb = AI851565/gi = 5495471/ug = Mm 10641/len = 341/STRA = for | | | | | | |
| 19 | 102332_at | Cluster Incl AI850194 UI-M-BG1-aie-a-01-0-UI s1 Mus musculus cDNA, 3 end/clone = UI-M-BG1-aie-a-01-0-UI/ | 260.5 | P | 273 | P | 314.9 | P |
| | | clone_end = 3/gb = AI850194/gi = 5494100/ug = Mm 10750/len = 294/STRA = rev | | | | | | |
| 19 | 102389_s_at | Cluster Incl AI841303: UI-M-AM0-adu-h-03-0-UI s1 Mus musculus cDNA, 3 end/clone = UI-M-AM0-adu-h-03-0-UI/ | 321.1 | P | 280.9 | P | 272.5 | P |
| | | clone_end = 3/gb = AI841303/gi = 5475516/ug = Mm 1222/len = 472/STRA = for | | | | | | |
| 19 | 102807_at | Cluster Incl AW048054 UI-M-BH1-alv-g-07-0-UI s1 Mus musculus cDNA, 3 end/clone = UI-M-BH1-alv-g-07-0-UI/ | 348.7 | P | 445.6 | P | 353.4 | P |
| | | clone_end = 3/gb = AW048054/gi = 5908583/ug = Mm 14155/len = 457/STRA = for | | | | | | |
| 19 | 102815_at | Cluster Incl U65986: Annexin XI/cds = (134,1645)/gb = U65986/gi = 1815638/ug = Mm 1427/len = 2370/STRA = for | 943.1 | P | 1269 | P | 1413.9 | P |
| 19 | 102835_at | Cluster Incl X14972 Mouse mRNA for alpha-adaptin (C)/cds = (150,2966)/gb = X14972/gi = 49879/ug = Mm 14555/len = 3242/ | 387.6 | P | 429.5 | P | 463.9 | P |
| | | STRA = for | | | | | | |
| 19 | 103002_at | Cluster Incl M27923 Glycoprotein galactosyltransferase beta 1, 4/cds = (216,1376)/gb = M27923/gi = 609527/ug = Mm.15622/ | 1711.9 | P | 1655.5 | P | 1559.8 | P |
| | | len = 1540/STRA = for | | | | | | |
| 19 | 103559_at | Cluster Incl M12303 Protein kinase, cAMP dependent, catalytic, alpha/cds = (186,1241)/gb = M12303/gi = 200366/ | 991.5 | P | 700.5 | P | 958 | P |
| | | ug = Mm 19111/len = 2231/STRA = for | | | | | | |
| 19 | 103584_at | Cluster Incl AW124334 UI-M-BH2.1-apq-f-05-0-UI s1 Mus musculus cDNA, 3 end/clone = UI-M-BH2 1-apq-f-05-0-UI/ | 1022.6 | P | 1032.8 | P | 935.5 | P |
| | | clone_end = 3/gb = AW124334/gi = 6099864/ug = Mm 19802/len = 227/STRA = rev | | | | | | |
| 19 | 104041_at | Cluster Incl AW122255 UI-M-BH2 2-aov-g-06-0-UI s1 Mus musculus cDNA, 3 end/clone = UI-M-BH2.2-aov-g-06-0-UI/ | 927.2 | P | 1084.4 | P | 1072.8 | P |
| | | clone_end = 3/gb = AW122255/gi = 6097722/ug = Mm 21458/len = 454/STRA = for | | | | | | |
| 19 | 104058_at | Cluster Incl AW047528 UI-M-BH1-ama-d-09-0-UI s1 Mus musculus cDNA, 3 end/clone = UI-M-BH1-ama-d-09-0-UI/ | 877.1 | P | 954.7 | P | 880.8 | P |
| | | clone_end = 3/gb = AW047528/gi = 5908057/ug = Mm 21537/len = 418/STRA = for | | | | | | |
| 19 | 104083_at | Cluster Incl AI853217 UI-M-BH0-ajh-f-11-0-UI s1 Mus musculus cDNA, 3 end/clone = UI-M-BH0-ajh-f-11-0-UI/ | 654.5 | P | 488.4 | P | 573.1 | P |
| | | clone_end = 3/gb = AI853217/gi = 5497123/ug = Mm 21767/len = 230/STRA = for | | | | | | |
| 19 | 104110_at | Cluster Incl AW060515: UI-M-BH1-ann-d-07-0-UI s1 Mus musculus cDNA, 3 end/clone = UI-M-BH1-ann-d-07-0-UI/ | 1035.5 | P | 1217.4 | P | 1196.9 | P |
| | | clone_end = 3/gb = AW060515/gi = 6008266/ug = Mm 21919/len = 330/STRA = for | | | | | | |
| 19 | 104121_at | Cluster Incl M90365 Junction plakoglobin/cds = (0,1865)/gb = M90365/gi = 1389681/ug = Mm 21990/len = 1878/STRA = rev | 2040.5 | P | 2777.8 | P | 2537.9 | P |
| 19 | 104139_at | Cluster Incl U16162 Mus musculus prolyl 4-hydroxylase alpha(l)-subunit mRNA, partial cds/cds = (0,1582)/gb = U16162/ | 187.5 | P | 221.9 | P | 187.9 | P |
| | | gi = 836897/ug = Mm 2212/len = 2129/STRA = for | | | | | | |
| 19 | 104145_at | Cluster Incl U81030 Treacher Collins Franceschetti syndrome 1, homolog/cds = (48,3956)/gb = U81030/gi = 2145148/ | 593.5 | P | 618.8 | P | 538.4 | P |
| | | ug = Mm 2215/len = 4428/STRA = for | | | | | | |
| 19 | 104554_at | Cluster Incl L25674: Mus musculus ear-2 transcription factor mRNA, complete cds/cds = (113,1282)/gb = L25674/gi = 409757/ | 439 | P | 550.4 | P | 569.9 | P |
| | | ug = Mm 2376/len = 1643/STRA = for | | | | | | |
| 19 | 104714_at | Cluster Incl AW125299 UI-M-BH2 1-apy-c-05-0-UI s1 Mus musculus cDNA, 3 end/clone = UI-M-BH2 1-apy-c-05-0-UI/ | 485.7 | P | 485 | P | 475.2 | P |
| | | clone_end = 3/gb = AW125299/gi = 6100829/ug = Mm 24477/len = 374/STRA = for | | | | | | |
| 19 | 94345_at | Cluster Incl AI843709: UI-M-AK1-aer-e-08-0-UI s1 Mus musculus cDNA, 3 end/clone = UI-M-AK1-aer-e-08-0-UI/ | 352.2 | P | 504.6 | P | 437.6 | P |
| | | clone_end = 3/gb = AI843709/gi = 5477922/ug = Mm 25142/len = 419/STRA = rev | | | | | | |
| 19 | 94357_at | Cluster Incl AA591002 vn61e07.r1 Mus musculus cDNA, 5 end/clone = IMAGE-1025700/clone_end = 5/gb = AA591002/ | 95.8 | P | 140.1 | P | 131.4 | P |
| | | gi = 2404315/ug = Mm.25237/len = 504/STRA = for | | | | | | |
| 19 | 95016_at | Cluster Incl D50086 Mouse mRNA for neuropilin, complete cds/cds = (347,3118)/gb = D50086/gi = 1783300/ug = Mm.27448/ | 1062 | P | 989.7 | P | 1352 | P |
| | | len = 3652/STRA for | | | | | | |
| 19 | 95023_at | Cluster Incl AW050353 UI-M-BH1-ang-d-12-0-UI.s1 Mus musculus cDNA, 3 end/clone = UI-M-BH1-ang-d-12-0-UI/ | 1872.5 | P | 1301.7 | P | 1841.9 | P |
| | | clone_end = 3/gb = AW050353/gi = 5910882/ug = Mm 27490/len = 514/STRA = for | | | | | | |
| 19 | 95602_at | Cluster Incl AI848135 UI-M-AP1-agk-h-05-0-UI s1 Mus musculus cDNA, 3 end/clone = UI-M-AP1-agk-h-05-0-UI/ | 1448.6 | P | 1568.5 | P | 1665.8 | P |
| | | clone_end = 3/gb = AI848135/gi = 5492041/ug = Mm 27942/len = 365/STRA = for | | | | | | |
| 19 | 96157_at | Cluster Incl AI839059 UI-M-AK0-adi-c-06-0-UI s1 Mus musculus cDNA, 3 end/clone = UI-M-AK0-adi-c-06-0-UI/ | 1265.1 | P | 1292.8 | P | 1548.4 | P |
| | | clone_end = 3/gb = AI839059/gi = 5473272/ug = Mm 28324/len = 514/STRA = for | | | | | | |
| 19 | 96748_i_at | Cluster Incl AA619554 vo88e08 r1 Mus musculus cDNA, 5 end/clone = IMAGE-1066214/clone_end = 5/gb = AA619554/ | 149.1 | P | 220.1 | P | 194.6 | P |
| | | gi = 2523430/ug = Mm 28958/len = 468/STRA = for | | | | | | |
| 19 | 96829_at | Cluster Incl AW047083 UI-M-BH1-alr-f-05-0-UI s1 Mus musculus cDNA, 3 end/clone = UI-M-BH1-alr-f-05-0-UI/ | 953.9 | P | 931.2 | P | 1029.9 | P |
| | | clone_end = 3/gb = AW047083/gi = 5907612/ug = Mm 29398/len = 311/STRA = rev | | | | | | |
| 19 | 97424_at | Cluster Incl AW049647 UI-M-BH1-and-g-07-0-UI s1 Mus musculus cDNA, 3 end/clone = UI-M-BH1-and-g-07-0-UI/ | 917 | P | 878.9 | P | 868.6 | P |
| | | clone_end = 3/gb = AW049647/gi = 5910176/ug = Mm 30009/len = 487/STRA = for | | | | | | |
| 19 | 98031_at | Cluster Incl AF027707 Mus musculus apoptosis activator Mtd (Mtd) mRNA, complete cds/cds = (188,829)/gb = AF027707/ | 724.8 | P | 769.3 | P | 827.2 | P |
| | | gi = 2689659/ug = Mm 3295/len = 1430/STRA = for | | | | | | |
| 19 | 99027_at | Cluster Incl L35049 Bcl2-like/cds = (242,943)/gb = L35049/gi = 561740/ug = Mm 3882/len = 979/STRA = for | 569.6 | P | 650.2 | P | 790.4 | P |
| 19 | 99034_at | Cluster Incl Y15001 Mus musculus mRNA for Iroquois homeobox protein 3/cds = (0,1523)/gb = Y15001/gi = 2765437/ | 727.8 | P | 595.7 | P | 892.3 | P |
| | | ug = Mm 39039/len = 1937/STRA = for | | | | | | |
| 19 | 100492_at | Cluster Incl AW122807: UI-M-BH2.1-aoy-h-10-0-UI s1 Mus musculus cDNA, 3 end/clone = UI-M-BH2 1-aoy-h-10-0-UI/ | 555.2 | P | 490 | P | 585 | P |
| | | clone_end = 3/gb = AW122807/gi = 6098337/ug = Mm 5063/len = 142/STRA = for | | | | | | |
| 19 | 100515_at | Cluster Incl X54056 Proprotein convertase subtillsin/kexin type 3/cds = (94,2475)/gb = X54056/gi = 50996/ug = Mm 5241/ | 1484 | P | 1398.8 | P | 1396 | P |
| | | len/3978/STRA = rev | | | | | | |
| 19 | 100986_at | Cluster Incl AF055889: Mus musculus LIM protein FHL2 (Fhl2) mRNA, complete cds/cds = (11,850)/gb = AF055889/ | 719.9 | P | 645 | P | 764.9 | P |
| | | gi = 3025852/ug = = Mm.6799/len = 1300/STRA = for | | | | | | |
| 19 | 101457_at | Cluster Incl L16956: Janus Kinase 2/cds = (93,3482)/gb = L16956/gi = 309463/ug = Mm 809/len = 3629/STRA = for | 157.9 | P | 153.1 | P | 130.5 | P |
| 19 | 101836_at | Cluster Incl D45859 Protein phosphatase 1B, magnesium dependent, beta isoform/cds = (0,461)/gb = D45B59/gi = 961469/ | 95.2 | P | 100.9 | P | 87.7 | P |
| | | ug = Mm 849/len = 546/STRA = for | | | | | | |
| 19 | 101876_s_at | Cluster Incl M35247 Histocompatibility 2, T region locus 17/cds = (34,168)/gb = M35247/gi = 199685/ug = Mm 88092/ | 324.6 | P | 297 | P | 576.3 | P |
| | | len = 1497/STRA = for | | | | | | |
| 19 | 101881_g_at | Cluster Incl L22545 Procollagen, type XVIII, alpha 1/cds = (0,3866)/gb = L22545/gi = 348968/ug = Mm 88304/len = 4031/ | 524.2 | P | 402.3 | P | 619 | P |
| | | STRA = rev | | | | | | |
| 19 | 102201_s_at | Cluster Incl L42177 Presenilin 1/cds = (187,1590)/gb = L42177/gi = 904129/ug = Mm 998/len = 1964/STRA = for | 1077.8 | P | 964.7 | P | 1085.5 | P |
| 19 | 102204_at | Cluster Incl L36435 Kreisler (maf-related) leucine zipper protein/cds = (169,1140)/gb = L35435/gi = 625043/ug = Mm 100216/ | 919.5 | P | 756 | P | 848.9 | P |
| | | len = 1458/STRA = for | | | | | | |
| 19 | 103527_at | Cluster Incl AW124360 UI-M-BH2 1-apq-h-07-0-UI.s1 Mus musculus cDNA, 3 end/clone = UI-M-BH2.1-apq-h-07-0-UI/ | 107.6 | P | 100.6 | P | 176.8 | P |
| | | clone_end = 3/gb = AW124360/gi = 6099890/ug = Mm 23011/len = 232/STRA = rev | | | | | | |
| 19 | 103542_at | Cluster Incl AW215059 up06a04 y1 Mus musculus cDNA, 5 end/clone = IMAGE-2651214/clone_end = 5/gb = AW215059/ | 1034.1 | P | 1094 | P | 920.7 | P |
| | | gi = 6525808/ug = Mm 23806/len = 245/STRA = rev | | | | | | |
| 19 | 104217_at | Cluster Incl AW045753 UI-M-BH1-akt-a-10-0-UI s1 Mus musculus cDNA, 3 end/clone = UI-M-BH1-akt-a-10-0-UI/ | 242 | P | 315.5 | P | 458.1 | P |
| | | clone_end = 3/gb = AW045753/gi = 5906282/ug = Mm.27893/len = 407/STRA = rev | | | | | | |
| 19 | 104221_at | Cluster Incl AB017189 Mus musculus mRNA for 4F2/CD98 light chain, complete cds/cds = (26,1564)/gb = AB017189/ | 1043.5 | P | 963.3 | P | 887.8 | P |
| | | gi = 4519255/ug = Mm 27943/len = 3441/STRA = for | | | | | | |
| 19 | 104241_at | Cluster Incl AI854379: UI-M-BG1-aic-f-11-0-UI s1 Mus musculus cDNA, 3 end/clone = UI-M-BG1-aic-f-11-0-UI/ | 1443.7 | P | 1302.5 | P | 1474.1 | P |
| | | clone_end = 3/gb = AI854379/gi = 5498285/ug = Mm.28184/len = 374/STRA = rev | | | | | | |
| 19 | 104514_at | Cluster Incl AF057285: Mus musculus intersectin-EH binding protein Ibp1 mRNA, partial cds/cds = (0,639)/gb = AF057285/ | 2334.4 | P | 2070.6 | P | 2263.8 | P |
| | | gi = 3063646/ug = Mm 3091/len = 1048/STRA = for | | | | | | |
| 19 | 92245_at | Cluster Incl AI642262 vn75a09 y1 Mus musculus cDNA, 5 end/clone = IMAGE-1037752/clone_end = 5/gb = AI642262/ | 247.3 | P | 299.3 | P | 429.5 | P |
| | | gi = 4720737/ug = Mm 35019/len = 255/STRA = rev | | | | | | |
| 19 | 93195_at | Cluster Incl AI957146 ul76b03 x1 Mus musculus cDNA, 3 end/clone = IMAGE-2136461/clone_end = 3/gb = AI957146/ | 342.1 | P | 359.6 | P | 458.7 | P |
| | | gi = 5749855/ug = Mm 5601/len = 465/STRA = for | | | | | | |
| 19 | 93624_at | Cluster Incl AI225296 ue86b01 y1 Mus musculus cDNA, 5 end/clone = IMAGE-1497961/clone_end = 5/gb = AI225296/ | 189.3 | P | 272.9 | P | 166.3 | P |
| | | gi = 3808357/ug = Mm 7446/len = 418/STRA = for | | | | | | |
| 19 | 93673_at | Cluster Incl U78109 Neurturin/cds = (348,935)/gb = U78109/gi = 1731678/ug = Mm 8074/len = 1023/STRA = for | 837.2 | P | 789 | P | 881.1 | P |
| 19 | 93712_at | Cluster Incl AF095640 Mus musculus cyclin T1 mRNA, complete cds/cds = (0,2174)/gb = AF095640/gi = 4106516/ | 407 | P | 467.8 | P | 366.8 | P |
| | | ug = Mm 86538/len = 2175/STRA = for | | | | | | |
| 19 | 94192_at | Cluster Incl Y17860 Mus musculus mRNA for ganglioside-Induced differentiation associated protein 10/cds = (2531,2917)/ | 326.9 | P | 306.7 | P | 386 | P |
| | | gb = Y17860/gi = 3378471/ug = Mm 12950/len = 5011/STRA = for | | | | | | |
| 19 | 94794_at | Cluster Incl X52561: Mouse gene for ferritin H subunit/cds = (166,714)/gb = X52561/gi = 50953/ug = Mm 1776/len = 860/ | 4730.7 | P | 4603 | P | 5370.7 | P |
| | | STRA = for | | | | | | |
| 19 | 96596_at | Cluster Incl U52073 Mus musculus TDD5 mRNA, complete cds/cds = (135,1280)/gb = U52073/gi = 1903378/ug = Mm 30837/ | 717.4 | P | 697.1 | P | 594.5 | P |
| | | len = 2916/STRA = for | | | | | | |
| 19 | 97786_at | Cluster Incl AJ011118 Mus musculus mRNA for a skeletal muscle and cardiac protein/cds = (10,996)/gb = AJ011118/ | 390.7 | P | 434.6 | P | 571.4 | P |
| | | gi = 5420271/ug = Mm 41992/len = 1100/STRA = for | | | | | | |
| 19 | 98868_at | Cluster Incl 31532 B-cell leukemia/lymphoma 2/cds = (1197,1796)/gb = L31532/gi = 468337/ug = Mm 5155/len = 1897/ | 168.9 | P | 123.8 | P | 162.1 | P |
| | | STRA = for | | | | | | |
| 19 | 99849_at | Cluster Incl C85523 C85523 Mus musculus cDNA, 3 end/clone = J0209F01/clone_end = 3/gb = C85523/gi = 2917480/ | 4570.3 | P | 5269 | P | 5958.3 | P |
| | | ug = Mm 63525/len = 566/STRA = rev | | | | | | |
| 19 | 100332_s_at | Cluster Incl AF093853 Mus musculus 1-Cys peroxiredoxin protein 2 gene, complete cds/cds = (0,674)/gb = AF093853/ | 419.5 | P | 453 | P | 429.9 | P |
| | | gi = 3769943/ug = Mm 87841/len = 675/STRA = for | | | | | | |
| 19 | 95796_g_at | Cluster Incl U96810 Mus musculus Sup14h2 gene, complete cds/cds = (0,353)/gb = U96810/gi = 3779195/ug = Mm 87825/ | 762.3 | P | 626.2 | P | 862.6 | P |
| | | len = 354/STRA = for | | | | | | |
| 19 | 96391_at | Cluster Incl C80836 C80836 Mus musculus cDNA, 3 end/clone = J0089A10/clone_end = 3/gb = C80836/gi = 2521166/ | 326.8 | P | 236 | P | 413.3 | P |
| | | ug = Mm.25089/len = 401/STRA = rev | | | | | | |
| 19 | 97601_f_at | Cluster Incl AW259538: up30a03 x1 Mus musculus cDNA, 3 end/clone = IMAGE-2655820/clone_end = 3/gb = AW259538/ | 3110.5 | P | 3013 | P | 3118.6 | P |
| | | gi = 6632519/ug = Mm 89688/len = 374/STRA = rev | | | | | | |
| 19 | 93492_at | Cluster Incl AB013469 Mus musculus CLM2 gene for cytohesin 2, complete and partial cds, alternative splicing/ | 1032.7 | M | 840.2 | P | 914.4 | P |
| | | cds = (0,1154)/gb = AB013469/gi = 3660542/ug = Mm 19097/len = 1155/STRA = for | | | | | | |
| 19 | 94299_at | Cluster Incl AI839731: UI-M-AP0-abe-a-03-0-UI s2 Mus musculus cDNA, 3 end/clone = UI-M-AP0-abe-a-03-0-UI/ | 255.3 | P | 162.4 | M | 321.3 | P |
| | | clone_end = 3/gb = AI839731/gi = 5473939/ug = Mm 22597/len = 359/STRA = for | | | | | | |
| 19 | 97915_at | Cluster Incl AW124144: UI-M-BH2 1-apr-e-12-0-UI s1 Mus musculus cDNA, 3 end/clone = UI-M-BH2 1-apr-e-12-0-UI/ | 482.4 | P | 544.1 | P | 544.3 | P |
| | | clone_end = 3/gb = AW124144/gi = 6099674/ug = Mm 28491/len = 442/STRA = for | | | | | | |
| 19 | 99677_at | Cluster Incl AI849559: UI-M-AH1-agr-b-09-0-UI s1 Mus musculus cDNA, 3 end/clone = UI-M-AH1-agr-b-09-0-UI/ | 393.7 | P | 366.9 | A | 398 | P |
| | | clone_end = 3/gb = AI849559/gi = 5493465/ug = Mm 44218/len = 371/STRA = for | | | | | | |
| 19 | 104562_at | Cluster lncl AW049632: UI-M-BH1-and-f-02-0-UI.s1 Mus musculus cDNA, 3 end/clone = UI-M-BH1-and-f-02-0-UI/ | 313.6 | P | 344.4 | P | 340.2 | P |
| | | clone_end = 3/gb = AW049632/gi = 5910181/ug = Mm 23838/len = 385/STRA = for | | | | | | |
| 19 | 96205_at | Cluster Incl AW048272: UI-M-BH1-aly-e-02-0-UI s1 Mus musculus cDNA, 3 end/clone = UI-M-BH1-aly-e-02-0-UI/ | 354.8 | P | 297.8 | P | 190.7 | P |
| | | clone_end = 3/gb = AW048272/gi = 5908801/ug = Mm.28638/len = 465/STRA = for | | | | | | |
| 19 | 97319_at | Cluster Incl AF084466 Mus muaculus Ras-like GTP-binding protein Red mRNA, complete cds/cds = (8,932)/gb = AF084466/ | 289.1 | P | 344.1 | P | 255.1 | P |
| | | gi = 3462897/ug = Mm 29467/len = 934/STRA = for | | | | | | |
| 19 | 97363_at | Cluster Incl M95408 Focal adhesion kinase/cds = (109,3267)/gb = M95408/gi = 193223/ug = Mm 29674/len = 4211/STRA = for | 54.4 | A | 47.8 | P | 96.9 | P |
| 19 | 97398_at | Cluster Incl AI849831: UI-M-AL1-aho-h-05-0-UI s1 Mus musculus cDNA, 3 end/clone = UI-M-AL1-aho-h-05-0-UI/ | 118.8 | P | 131.1 | P | 220.8 | P |
| 19 | 97413_at | Cluster Incl AI121305: uc30b06.r1 Mus musculus cDNA, 5 end/clone = IMAGE-1399475/clone_end = 5/gb = AI121305/ | 743.4 | P | 863.2 | P | 732.7 | P |
| | | gi = 3521629/ug = Mm.29959/len = 358/STRA = for | | | | | | |
| 19 | 98472_at | Cluster Incl Y00629: Histocompatibility 2, T region locus 23/cds = (32,1105)/gb = Y00629/gi = 51013/ug = Mm.35016/ | 358.5 | P | 316.6 | P | 797.6 | P |
| | | len = 1282/STRA = for | | | | | | |
| 19 | 102627_at | Cluster Incl AF061569 Mus musculus coding region determinant binding protein mRNA, complete cds/cds = (130,1863)/ | 272.4 | P | 206.6 | P | 206.1 | P |
| | | gb = AF061569/gi = 3273748/ug = Mm 12919/len = 2223/STRA = for | | | | | | |
| 19 | 104201_at | Cluster Incl AA718649 vt86g08 r1 Mus musculus cDNA, 5 end/clone = IMAGE-1178078/clone_end = 5/gb = AA718649/ | 834.6 | P | 624.6 | P | 842.6 | P |
| | | gi = 2730923/ug = Mm 27525/len = 479/STRA = for | | | | | | |
| 19 | 92189_at | Cluster Incl AI846017: UI-M-AP1-agl-f-08-0-UI s1 Mus musculus cDNA, 3 end/clone = UI-M-AP1-agl-f-08-0-UI/clone_end = 3/ | 184.6 | A | 224.6 | P | 235.6 | P |
| 19 | 99397_at | Cluster Incl U97572 Excision repair 2/cds = (0,2282)/gb = U97572/gi = 2114483/ug = Mm 56990/len = 2283/STRA = for | 412.4 | P | 391.2 | P | 479.3 | P |
| 19 | 99915_at | Cluster Incl L41352 Amphiregulin/cds = (0,746)/gb = L41352/gi = 845598/ug = Mm.8039/len = 747/STRA = for | 125.8 | P | 95.8 | P | 127 | P |
| 19 | 101676_at | Cluster Incl U13705 Glutathione peroxidase 3/cds = (16,696)/gb = U13705/gi = 536927/ug = Mm 7156/len = 1415/STRA = for | 557.2 | A | 665.5 | P | 791.5 | P |
| 19 | 95095_at | Cluster Incl U90435 Mus musculus flotillin mRNA, complete cds/cds = (159,1445)/gb = U90435/gi = 2149603/ug = Mm.2931/ | 18.2 | P | −61.4 | P | 229.6 | P |
| | | len = 1702/STRA = for | | | | | | |
| 19 | 101014_at | Cluster Incl Y09864 M musculus mRNA for type I interferon receptor, IFNaR2b/cds = (220,963)/gb = Y09864/gi = 2467245/ | 65 | P | 95.1 | P | 139.8 | P |
| | | ug = Mm 6834/len = 2586/STRA = for | | | | | | |
| 19 | 103053_at | Cluster Incl X15784 Myogenin/cds = (48,788)/gb = X15784/gi = 53303/ug = Mm 16528/len = 1474/STRA = for | 327.1 | P | 263.1 | P | 397.4 | P |
| 19 | 103335_at | Cluster Incl U55060 Mus musculus beta-galactoside binding lectin mRNA, complete cds/cds = (34,1002)/gb = U55060/ | 809.3 | P | 740.2 | P | 948.7 | P |
| | | gi = 1916601/ug = Mm 18087/len = 1418/STRA = for | | | | | | |
| 19 | 103557_at | Cluster Incl AI850046 UI-M-BG0-aia-b-11-0-U1 s1 Mus musculus cDNA, 3 end/clone = UI-M-BG0-aia-b-11-0-UI/ | 419.7 | P | 480 | P | 417.4 | P |
| | | clone_end = 3/gb = AI850046/gi = 5493952/ug = Mm 19091/len = 484/STRA = for | | | | | | |
| 19 | 104152_at | Cluster Incl AA960466 ub58f11 s1 Mus musculus cDNA, 3 end/clone = IMAGE-1381965/clone_end = 3/gb = AA960466/ | 1248.1 | P | 1083.5 | P | 1541.1 | P |
| | | gi = 3126366/ug = Mm 22193/len = 202/STRA = for | | | | | | |
| 19 | 103737_at | Cluster Incl AB016780 Mus musculus mRNA for Glutamine-fructose-6-phosphate amidotransferase 2, complete cds/ | 107.7 | M | 266.7 | P | 450.4 | P |
| | | cds = (48,2096)/gb = AB016780/gi = 4239878/ug = Mm 24402/len = 2958/STRA = for | | | | | | |
| 19 | 104645_at | Cluster Incl AI853712.UI-M-BH0-ajq-d-05-0-UI s1 Mus musculus cDNA, 3 end/clone = UI-M-BH0-ajq-d-05-0-UI/ | 363.8 | P | 297.3 | A | 351.8 | P |
| | | clone_end = 3/gb = AI853712/gi = 5497583/ug = Mm 31321/len = 496/STRA = rev | | | | | | |
| 19 | 93865_s_at | Cluster Incl M35244: Histocompatibility 2, T region locus 10/cds = UNKNOWN/gb = M35244/gi = 199063/ug = Mm.87776/ | 392.5 | P | 273.5 | A | 581 | P |
| | | len = 1714/STRA = for | | | | | | |
| 19 | 95910_f_at | Cluster Incl AI844780 UI-M-AL1-ahq-g-08-0-UI s1 Mus musculus cDNA, 3 end/clone = UI-M-AL1-ahq-g-08-0-UI/ | 306.7 | P | 259.7 | P | 283.7 | P |
| | | clone_end = 3/gb = AI844780/gi = 5488686/ug = Mm 24440/len = 200/STRA = for | | | | | | |
| 19 | 93283_at | Cluster Incl AA869401 vq07f04 r1 Mus musculus cDNA, 5 end/clone = IMAGE-1093567/clone_end = 5/gb = AA869401/ | 600.1 | P | 326.5 | P | 656.8 | P |
| | | gi = 2964846/ug = Mm 1786/len = 793/STRA = for | | | | | | |
| 19 | 96646_at | Cluster Incl AI846253 UI-M-AK1-aez-b-10-0-UI s1 Mus musculus cDNA, 3 end/clone = UI-M-AK1-aez-b-10-0-UI/ | 224.5 | P | 170.9 | P | 234.7 | P |
| | | clone_end = 3/gb = AI846253/gi = 5490159/ug = Mm 27292/len = 311/STRA = for | | | | | | |
| 19 | 99100_at | Cluster Incl AI837104 UI-M-AK0-adc-d-07-0-UI s1 Mus musculus cDNA, 3 end/clone = UI-M-AK0-adc-d-07-0-UI/ | 543.8 | P | 690.1 | P | 670.5 | P |
| | | clone_end = 3/gb = AI837104/gi = 5471317/ug = Mm 3948/len = 413/STRA = for | | | | | | |
| 19 | 101956_at | Cluster Incl AI834849 UI-M-AN0-acm-d-08-0-UI s2 Mus musculus cDNA, 3 end/clone = UI-M-AN0-acm-d-08-0-UI/ | 855.5 | P | 774.7 | P | 910.4 | P |
| | | clone_end= 3/gb = AI834849/gi = 5469062/ug = Mm 9210/len = 176/STRA = for | | | | | | |
| 19 | 102849_at | Cluster Incl D88159 Potassium inwardly rectifying channel, subfamily J, member 8/cds = (203,1477)/gb = D88159/ | 67.3 | A | 156.9 | P | 116.5 | P |
| | | gi = 1881405/ug = Mm 1482/len = 1712/STRA = for | | | | | | |
| 19 | 101934_at | Cluster Incl AI851119 UI-M-BH0-ajx-a-08-0-UI s1 Mus musculus cDNA, 3 end/clone = UI-M-BH0-ajx-a-08-0-UI/ | 117.4 | P | 78.4 | P | 80.2 | P |
| | | clone_end = 3/gb = AI851119/gi = 5495065/ug = Mm.946/len = 354/STRA = for | | | | | | |
| 19 | 94147_at | Cluster Incl M33960: Mouse plasminogen activator inhibitor (PAI-1) mRNA, complete cds/cds = (131,1339)/gb = M33960/ | 153.5 | P | 195.1 | P | 257.6 | P |
| | | gi = 200219/ug = Mm.1263/len = 2999/STRA = for | | | | | | |
| 19 | 96418_r_at | Cluster Incl M18399 Mouse homeo box (Hox2.4) region/cds = (0,389)/gb = M18399/gi = 193940/ug = Mm 88400/len = 390/ | 560.5 | M | 580.3 | P | 652 | M |
| | | STRA = for | | | | | | |
| 19 | 93352_at | Cluster Incl M55154.Mouse transglutaminase (TGase) mRNA, complete cds/cds = (29,2086)/gb = M55154/gi = 201940/ | 213.1 | A | 193 | A | 187.5 | P |
| | | ug = Mm 18843/len = 3483/STRA = for | | | | | | |
| 19 | 100552_at | Cluster Incl M28233: Mouse interferon-gamma receptor mRNA, complete cds/cds = (101,1534)/gb = M28233/gi = 194131/ | 165.8 | P | 181.5 | P | 249.2 | P |
| | | ug = Mm 549/len = 2093/STRA = for | | | | | | |
| 19 | 102845_at | Cluster Incl A1836034 UI-M-AQ0-aad-f-06-0-UI s2 Mus musculus cDNA, 3 end/clone = Ul-M-AQ0-aad-f-06-0-UI/ | 487 | P | 368.1 | A | 448 | P |
| | | clone_end = 3/gb = AI838034/gi = 5470212/ug = Mm 14741/len = 389/STRA = rev | | | | | | |
| 19 | 102988_at | Cluster Incl U92477: Mus musculus AblSH3 binding protein mRNA, partial cds/cds = (0,1440)/gb = U92477/gi = 1928963/ | 689.7 | A | 779.9 | M | 997.5 | P |
| | | ug = Mm 1531/len = 2188/STRA = for | | | | | | |
| 19 | 104548_at | Cluster Incl Y15443 Mus musculus mRNA for 50C15 protein/cds = (20,454)/gb = Y15443/gi = 2624230/ug = Mm 23697/ | 275.3 | A | 260.9 | P | 240.6 | A |
| | | len = 721/STRA = for | | | | | | |
| 19 | 94423_at | Cluster Incl AW049551 UI-M-BH1-anf-f-04-0-UI s1 Mus musculus cDNA, 3 end/clone UI-M-BH1-anf-f-04-0-UI/ | 153.4 | P | 83.8 | P | 85.2 | P |
| | | clone_end = 3/gb = AW049551/gi = Mm 26282/len = 447/STRA = for | | | | | | |
| 19 | 94997_at | Cluster Incl AF060883 Mus musculus endomucin mRNA, complete cds/cds = (97,843)/gb = AF060883/gi = 4159992/ | 240.5 | P | 215.7 | M | 234 | P |
| | | ug = Mm.27343/len = 1374/STRA = for | | | | | | |
| 19 | 96167_at | Cluster Incl AI643420: vv24h01.x1 Mus musculus cDNA, 3 end/clone = IMAGE-1223377/clone_end = 3/gb = AI643420/ | 569.8 | P | 572 | A | 585 | P |
| | | gi = 4721895/ug = Mm 28373/len = 195/STRA = for | | | | | | |
| 19 | 104268_at | Cluster Incl X51975: Interleukin 6 receptor, alpha/cds = (0,1382)/gb = X51975/gi = 49725/ug = Mm 2856/len = 2499/STRA = for | 186.5 | P | 437.6 | P | 681.7 | P |
| 19 | 99916_at | Cluster Incl D90242: Protein kinase C, eta/cds = (59,2110)/gb = D90242/gi = 220526/ug = Mm 8040/len = 2176/STRA = for | 49 | P | 106.8 | A | 88.9 | P |
| 19 | 94288_at | Cluster Incl J03482 Mouse histone H1 gene, complete cds/cds = (42,680)/gb = J03482/gi = 193847/ug = Mm 22570/len = 725/ | 996.1 | P | 667.6 | A | 569.4 | A |
| | | STRA = for | | | | | | |
| 19 | 102430_at | Cluster Incl X51397: Myeloid differentiation primary response gene 88/cds = (109,840)/gb = X51397/gi = 53293/ | 178.7 | A | 235.3 | A | 239.9 | P |
| | | ug = Mm.12847/len = 1805/STRA = for | | | | | | |
| 19 | 103367_at | Cluster Incl U18975 Ganglioside expression 2/cds = (3,1604)/gb = U18975/gi = 945034/ug = Mm 1853/len = 2109/STRA = for | 53.1 | M | 35.7 | P | 124.2 | P |
| 19 | 93108_at | Cluster Incl J05185 Prolyl 4-hydroxylase, beta polypeptide/cds = (129,1658)/gb = J05185/gi = 200280/ug = Mm.16660/ | 456.6 | P | 303.7 | P | 424 | M |
| | | len = 2537/STRA = for | | | | | | |
| 19 | 94902_at | Cluster Incl U38261 Mus musculus extracellular superoxide dismutase (SOD3) mRNA, complete cds/cds = (269,1024)/ | 418.1 | A | 485.1 | P | 532.5 | P |
| | | gb = U38261/gi = 1915962/ug = Mm 2407/len = 1834/STRA = for | | | | | | |
| 19 | 97830_at | Cluster Incl L06039 Platelet/endothelial cell adhesion molecule/cds = (98,2281)/gb = L06039/gi = 453288/ug = Mm.2822/ | 318.6 | P | 237 | A | 290.4 | P |
| | | len = 2534/STRA = for | | | | | | |
| 19 | 102046_at | Cluster Incl AF041847 Mus musculus cardiac ankynn repeal protein MCARP mRNA, complete cds/cds = (14,973)/ | 59.7 | P | 68.9 | P | 34.3 | M |
| | | gb = AF041847/gi = 2905615/ug = Mm 10279/len = 1026/STRA = for | | | | | | |
| 19 | 103068_at | Cluster Incl U68535: Mus musculus aldo-keto reductase mRNA, complete cds/cds = (27,932)/gb = U68535/gi = 1698717/ | 88.6 | A | 77.6 | P | 95.1 | P |
| | | ug = Mm 16790/len = 1675/STRA = for | | | | | | |
| 19 | 102195_at | Cluster Incl U88984: Mus musculus NIK mRNA, complete cds/cds = (0,3701)/gb = U88984/gi = 1872545/ug = Mm 987/ | 136.7 | A | 212 | P | 219.2 | P |
| | | len = 3915/STRA = rev | | | | | | |
| 19 | 103213_at | Cluster Incl AJ245402: Mus musculus partial cdh15 gene for cadherin/cds = (0,239)/gb = AJ245402/gi = 6010257/ | 121.4 | P | 100.5 | A | 122.1 | P |
| | | ug = Mm 1976/len = 240/STRA = for | | | | | | |
| 19 | 92738_at | Cluster Incl D49921: Mouse mRNA for glial cell line-derived neurotrophic factor(GDNF), complete cds/cds = (0,635)/ | 179.4 | A | 216.7 | A | 221.2 | A |
| | | gb = D49921/gi = 758584/ug = Mm 4679/len = 641/STRA = for | | | | | | |
| 19 | 97166_at | Cluster Incl W49316: mc89e12 r1 Mus musculus cDNA, 5 end/clone = IMAGE-355726/clone_end = 5/gb = W49316/ | 15.4 | P | 51.6 | A | 47.8 | P |
| | | gi = 1337600/ug = Mm.35552/len = 475/STRA = rev | | | | | | |
| 19 | 94643_at | Cluster Incl U35836: Tumor-associated antigen 1/cds = (10,1257)/gb = U35836/gi = 1017751/ug = Mm 8071/len = 1338/ | 90.2 | P | 69.2 | A | 100.3 | A |
| | | STRA = for | | | | | | |
| 20 | 92540_f_at | Cluster Incl Z67748 M musculus spermidine synthase gene/cds = (75,983)/gb = Z67748/gi = 1061191/ug = Mm.10/len = 1261/ | 196.2 | P | 437 | P | 363.5 | P |
| | | STRA = for | | | | | | |
| 20 | 92770_at | Cluster Incl X66449 Calcyclin/cds = (159,428)/gb = X66449/gi = 50271/ug = Mm 1221/len = 551/STRA = for | 2238.8 | P | 5325.6 | P | 6393.2 | P |
| 20 | 92817_at | Cluster Incl AW120646 UI-M-BH2 3-anz-f-08-0-UI s1 Mus musculus cDNA, 3 end/clone = UI-M-BH2.3-anz-f-08-0-UI/ | 753.4 | P | 652.9 | P | 907.3 | P |
| | | clone_end = 3/gb = AW120646/gi = 6096019/ug = Mm 12864/len = 434/STRA = for | | | | | | |
| 20 | 92861_i_at | Cluster Incl M13805 Mouse type I epidermal keratin mRNA, clone pkSCC-50, 3 end/cds = (0,617)/gb = M13805/gi = 198632/ | 2877.3 | P | 4444.7 | P | 3848.1 | P |
| | | ug = Mm 14046/len = 801/STRA = for | | | | | | |
| 20 | 92862_f_at | Cluster Incl M13805 Mouse type I epidermal keratin mRNA, clone pkSCC-50, 3 end/cds-(0,617)/gb = M13805/gi = 198632/ | 2163.7 | P | 5693 | P | 5813.7 | P |
| | | ug = Mm.14046/len = 801/STRA = for | | | | | | |
| 20 | 92877_at | Cluster Incl L19932 Transforming growth factor, beta induced, 68 kDa/cds = (45,2096)/gb = L19932/gi = 451131/ | 409.9 | P | 914.8 | P | 778.5 | P |
| | | ug = Mm 14455/len = 2674/STRA = for | | | | | | |
| 20 | 93018_at | Cluster Incl AI790103 ue64f04 r1 Mus musculus cDNA, 5 end/clone = IMAGE-1495903/clone_end = 5/gb = AI790103/ | 455.4 | P | 1114.3 | P | 891.7 | P |
| | | gi = 5337819/ug = Mm 14747/len = 346/STRA = for | | | | | | |
| 20 | 93042_at | Cluster Incl D21207 Benzodiazepine receptor, peripheral/cds = (65,574)/gb = D21207/gi = 484053/ug = Mm 1508/len = 856/ | 1719 | P | 1958 | P | 2277.7 | P |
| | | STRA = for | | | | | | |
| 20 | 93078_at | Cluster Incl X04653 Mouse mRNA for Ly-6 alloantigen (Ly-6E 1)/cds = (88,492)/gb = X04653/gi = 52958/ug = Mm 15889/ | 1292.2 | P | 2749.5 | P | 3605 | P |
| | | len = 876/STRA = for | | | | | | |
| 20 | 93264_at | Cluster Incl AI843895 UI-M-AK1-aeu-f-09-0-UI s1 Mus musculus cDNA, 3 end/clone = UI-M-AK1-aeu-f-09-0-UI/ | 597.1 | P | 820.1 | P | 963.8 | P |
| | | clone_end = 3/gb = AI843895/gi = 5478108/ug = Mm 17253/len = 420/STRA = for | | | | | | |
| 20 | 93288_at | Cluster Incl AI835883 UI-M-AI0-aat-f-08-0-UI.s1 Mus musculus cDNA, 3 end/clone = UI-M-AI0-aat-f-08-0-UI/clone_end = 3/ | 2961.5 | P | 3495.1 | P | 3815.6 | P |
| | | gb = AI835883/gi = 5470096/ug = Mm 17912/len = 426/STRA = for | | | | | | |
| 20 | 93362_at | Cluster Incl U27106 Adapter-related protein complex AP-2, mu1/cds = (141,1448)/gb = U27106/gi = 2065471/ug = Mm.18946/ | 1906.8 | P | 2106.5 | P | 1994.4 | P |
| | | len = 2066/STRA = rev | | | | | | |
| 20 | 93573_at | Cluster Incl V00835 Metallothionein 1/cds = (0,185)/gb = V00835/gi = 53247/ug = Mm 2041/len = 186/STRA = for | 1915 | P | 3661.8 | P | 3054.6 | P |
| 20 | 93586_at | Cluster Incl AJ002307: Mus musculus mRNA for synaptogyrin 2/cds = (4,678)/gb = AJ002307/gi = 2959869/ug = Mm.2079/ | 130.5 | P | 320.5 | P | 402.9 | P |
| | | len = 1512/STRA = for | | | | | | |
| 20 | 93605_r_at | Cluster Incl AF061260 Mus musculus immunosuperfamily protein B12 mRNA, complete cds/cds = (308,1195)/ | 145.6 | P | 249.9 | P | 233.3 | P |
| | | gb = AF081260/gi = 3779241/ug = Mm 20921/len = 1861/STRA = for | | | | | | |
| 20 | 93797_g_at | Cluster Incl AW123952 UI-M-BH2 3-aqc-d-05-0-UI s1 Mus musculus cDNA, 3 end/clone = UI-M-BH2 3-aqc-d-05-0-UI/ | 210.4 | P | 625.7 | P | 611 | P |
| | | clone_end = 3/gb = AW123952/gi = 6099482/ug = Mm 2140/len = 370/STRA = for | | | | | | |
| 20 | 94011_at | Cluster Incl AI845182 UI-M-BG0-aht-a-11-0-UI s1 Mus musculus cDNA, 3 end/clone = UI-M-BG0-aht-a-11-0-UI/ | 768.5 | P | 1714.7 | P | 1897 | P |
| | | clone_end = 3/gb = AI845182/gi = 5489088/ug = Mm 21819/len = 325/STRA = for | | | | | | |
| 20 | 94043_at | Cluster Incl AB031290 Mus musculus mRNA for C7-1 protein, complete cds/cds = (0,1391)/gb = AB031290/gi = 5771446/ | 525.4 | P | 556.1 | P | 602.9 | P |
| | | ug = Mm 21961/len = 1524/STRA = for | | | | | | |
| 20 | 94054_at | Cluster Incl AI841808: UI-M-AN0-acl-h-05-0-UI s1 Mus musculus cDNA, 3 end/clone = UI-M-AN0-acl-h-05-0-UI/ | 750.9 | P | 981.6 | P | 996.9 | P |
| | | clone_end = 3/gb = AI841808/gi = 5476021/ug = Mm 22024/len = 173/STRA = for | | | | | | |
| 20 | 94302_at | Cluster Incl AF013099 Mus musculus multiubiquitin-chain-binding protein (Mcb1) mRNA, complete cds/cds = (12,1142)/ | 851.5 | P | 883 | P | 1241 | P |
| | | gb = AF013099/gi = 2293572/ug = Mm 2261/len = 1252/STRA = for | | | | | | |
| 20 | 94826_at | Cluster Incl Y11460 Mus musculus mRNA for b4 Integnn interactor, partial/cds = (0,254)/gb = Y11460/gi = 2910994/ | 1022.8 | P | 1095.8 | P | 1293.5 | P |
| | | ug = Mm 22731/len = 438/STRA = for | | | | | | |
| 20 | 94831_at | Cluster Incl M65270 Mouse cathepsin B gene/cds = (25,1044)/gb = M65270/gi = 309151/ug = Mm.22753/len = 1848/ | 1860.8 | P | 2792.7 | P | 2994.4 | P |
| | | STRA = for | | | | | | |
| 20 | 94835_f_at | Cluster Incl M28739 Tubulin, beta 2/cds = (0,302)/gb = M28739/gi = 202226/ug = Mm 22774/len = 488/STRA = for | 2015.9 | P | 2744.7 | P | 2026.5 | P |
| 20 | 94837_at | Cluster Incl U67328 Mus musculus NIPI-like protein (NIPIL(A3)) mRNA, complete cds/cds = (466,1437)/gb = U67328/ | 2530.1 | P | 3258.8 | P | 3339.7 | P |
| | | gi = 1762985/ug = Mm 22776/len = 1720/STRA = for | | | | | | |
| 20 | 95406_at | Cluster Incl AW125347 UI-M-BH2 1-apy-h-03-0-UI s1 Mus musculus cDNA, 3 end/clone = UI-M-BH2 1-apy-h-03-0-UI/ | 844.5 | P | 1329.5 | P | 1492.4 | P |
| | | clone_end = 3/gb = AW125347/gi = 6100877/ug = Mm 24219/len = 331/STRA = for | | | | | | |
| 20 | 95417_at | Cluster Incl AI117848: uc41c02.r1 Mus musculus cDNA, 5 end/clone = IMAGE-1400546/clone_end = 5/gb = AI117848/ | 250 | P | 349.9 | P | 297.1 | P |
| | | gi = 3518172/ug = Mm 24293/len = 431/STRA = for | | | | | | |
| 20 | 95433_at | Cluster Incl AW049776 UI-M-BH1-anm-c-12-0-UI s1 Mus musculus cDNA, 3 end/clone = UI-M-BH1-anm-c-12-0-UI/ | 320.9 | P | 409.8 | P | 385.4 | P |
| | | clone_end = 3/gb = AW049776/gi = 5910305/ug = Mm.24495/len = 403/STRA = for | | | | | | |
| 20 | 95511_at | Cluster Incl X69902 Integrin alpha 6/cds = (181,3402)/gb = X69902/gi = 408127/ug = Mm 25232/len = 3756/STRA = for | 231.3 | P | 441.2 | P | 345.3 | P |
| 20 | 96033_at | Cluster Incl Z22532 Syndecan 1/cds = (240,1175)/gb = Z22532/gi = 1946227/ug = Mm 2580/len = 3054/STRA = for | 270.2 | P | 578.3 | P | 532.3 | P |
| 20 | 96042_at | Cluster Incl L35528: Mus musculus manganese superoxide dismutase (MnSOD) gene/cds = (70,738)/gb = L35528/ | 212.3 | P | 420 | P | 376.4 | P |
| | | gi = 2182263/ug = Mm 2597/len = 906/STRA = for | | | | | | |
| 20 | 96075_at | Cluster Incl AW060876 UI-M-BH1-amx-g-07-0-UI s1 Mus musculus cDNA, 3 end/clone = UI-M-BH1-amx-g-07-0-UI/ | 1101.4 | P | 1308 | P | 1343 | P |
| | | clone_end = 3/gb = AW060876/gi = 6008627/ug = Mm 2654/len = 472/STRA = for | | | | | | |
| 20 | 96092_at | Cluster Incl M96827 C57BL/6J ob/ob haptoglobin mRNA, complete cds/cds = (48,1091)/gb = M96827/gi = 193753/ | 555.3 | P | 4345.5 | P | 4203.6 | P |
| | | ug = Mm 26730/len = 1267/STRA = for | | | | | | |
| 20 | 96603_at | Cluster Incl AW123556 UI-M-BH2 1-aqb-g-04-0-UI s1 Mus musculus cDNA, 3 end/clone = UI-M-BH2 1-aqb-g-04-0-UI/ | 275.7 | P | 348.8 | P | 441 | P |
| | | clone_end = 3/gb = AW123556/gi = 6099086/ug = Mm 27035/len = 370/STRA = for | | | | | | |
| 20 | 96623_at | Cluster Incl AI853172 UI-M-BH0-ajh-b-06-0-UI s1 Mus musculus cDNA, 3 end/clone = UI-M-BH0-ajh-b-06-0-UI/ | 277.7 | P | 546.2 | P | 571.5 | P |
| | | clone_end = 3/gb = AI853172/gi = 5497078/ug = Mm 27173/len = 435/STRA = for | | | | | | |
| 20 | 96650_at | Cluster Incl AI837724: UI-M-AK0-adj-f-11-0-UI s1 Mus musculus cDNA, 3 end/clone = UI-M-AK0-adj-f-11-0-UI/clone_end = 3/ | 186.8 | P | 348.9 | P | 361.4 | P |
| | | gb = AI837724/gi = 5471937/ug = Mm 2733/len = 370/STRA = for | | | | | | |
| 20 | 96657_at | Cluster Incl L10244 Spermidine/spermine N1-acetyl transferase/cds = (89,604)/gb = L10244/gi = 309503/ug = Mm 2734/ | 459.9 | P | 1554.3 | P | 1848.5 | P |
| | | len = 961/STRA = for | | | | | | |
| 20 | 96704_at | Cluster Incl AF058798 Mus musculus 14-3-3 protein sigma mRNA, complete cds/cds = (163,909)/gb = AF058798/ | 3862.2 | P | 5049.1 | P | 4904.9 | P |
| | | gi = 3065926/ug = Mm 2758/len = 1430/STRA = for | | | | | | |
| 20 | 96718_at | Cluster Incl AB012727 Mus musculus C184L-22 mRNA, complete cds/cds = UNKNOWN/gb = AB012727/gi = 6469043/ | 767 | P | 535.7 | P | 696.2 | P |
| | | ug = Mm 27657/len = 2532/STRA = for | | | | | | |
| 20 | 97199_at | Cluster Incl AI854767 UI-M-BH0-aka-c-06-0-UI s1 Mus musculus cDNA, 3 end/clone = UI-M-BH0-aka-c-06-0-UI/ | 422 | P | 627.2 | P | 724.4 | P |
| | | clone_end = 3/gb = AI854767/gi = 5498673/ug = Mm 27660/len = 203/STRA = for | | | | | | |
| 20 | 97803_at | Cluster Incl U38196 Membrane protein, palmitoylated (55 kDa)/cds = (180,1580)/gb = U38196/gi = 1616986/ug = Mm 2814/ | 555.8 | P | 688 | P | 818.4 | P |
| | | len = 1976/STRA = for | | | | | | |
| 20 | 97875_at | Cluster Incl AW123694: UI-M-BH2.1-apl-c-08-0-UI s1 Mus musculus cDNA, 3 end/clone = UI-M-BH2 1-apl-c-08-0-UI/ | 395.5 | P | 599 | P | 575.3 | P |
| | | clone_end = 3/gb = AW123694/gi = 6099224/ug = Mm 28353/len = 479/STRA = for | | | | | | |
| 20 | 97882_at | Cluster Incl AB032902: Mus musculus Sec61 mRNA, complete cds/cds = ( 120,1550)/gb = AB032902/gi = 6012185/ | 949.5 | P | 1244.5 | P | 1425.2 | P |
| | | ug = Mm 28375/len = 1804/STRA = rev | | | | | | |
| 20 | 97890_at | Cluster Incl AW046181: UI-M-BH1-akw-d-06-0-UI s1 Mus musculus cDNA, 3 end/clone = UI-M-BH1-akw-d-06-0-UI/ | 841.8 | P | 1522.4 | P | 1763.5 | P |
| | | clone_end = 3/gb = AW046181/gi = 5906710/ug = Mm 28405/len = 376/STRA = for | | | | | | |
| 20 | 97914_at | Cluster Incl D17666 Mouse gene for mitochondrial stress-70 protein (PBP74/CSA)/cds = (73,2112)/gb = D17666/gi = 903308/ | 811.5 | P | 1194.8 | P | 1237.3 | P |
| | | ug = Mm 2849/len = 2998/STRA = for | | | | | | |
| 20 | 98911_at | Cluster Incl AI837528: UI-M-AL0-abs-b-09-0-UI.s1 Mus musculus cDNA, 3 end/clone = UI-M-AL0-abs-b-09-0-UI/ | 155.5 | P | 271.7 | P | 254.1 | P |
| | | clone_end = 3/gb = AI837528/gi = 5471741/ug = Mm 28598/len = 340/STRA = for | | | | | | |
| 20 | 94445_at | Cluster Incl AW125273: UI-M-BH2.1-aps-h-07-0-UI s1 Mus musculus cDNA, 3 end/clone = UI-M-BH2 1-aps-h-07-0-UI/ | 948.1 | P | 1782.1 | P | 1525 | P |
| | | clone_end = 3/gb = AW125273/gi = 6100803/ug = Mm 28777/len = 414/STRA = for | | | | | | |
| 20 | 94450_at | Cluster Incl AI854202 UI-M-BH0-ajz-d-08-0-UI.s1 Mus musculus cDNA, 3 end/clone = UI-M-BH0-ajz-d-08-0-UI/ | 559.4 | P | 684.2 | P | 642.8 | P |
| | | clone_end = 3/gb = AI854202/gi = 5498108/ug = Mm 28786/len = 543/STRA = for | | | | | | |
| 20 | 94486_at | Cluster Incl AW125178: UI-M-BH2.1-apz-g-02-0-UI s1 Mus musculus cDNA, 3 end/clone = UI-M-BH2 1-apz-g-02-0-UI/ | 1071.3 | P | 1531.1 | P | 1608.2 | P |
| | | clone_end = 3/gb = AW125178/gi = 6100708/ug = Mm 28892/len = 249/STRA = for | | | | | | |
| 20 | 94492_at | Cluster Incl AB025406 Mus musculus mRNA for sid23p, complete cds/cds = (34,531)/gb = AB025406/gi = 5931560/ | 3003.4 | P | 4629.8 | P | 4886.8 | P |
| | | ug = Mm 28919/len = 1639/STRA = for | | | | | | |
| 20 | 95079_at | Cluster Incl M57683 Platelet derived growth factor receptor, alpha polypeptide/cds = (78,3347)/gb = M57683/gi = 199783/ | 215.7 | P | 808.8 | P | 1037.7 | P |
| | | ug = Mm 2924/len = 3564/STRA = for | | | | | | |
| 20 | 95102_at | Cluster Incl AW123754 UI-M-BH2.1-apm-a-08-0-UI s1 Mus musculus cDNA, 3 end/clone = UI-M-BH2.1-apm-a-08-0-UI/ | 915 | P | 1216.4 | P | 1668.5 | P |
| | | clone_end = 3/gb = AW123754/gi = 6099284/ug = Mm 29345/len = 395/STRA = for | | | | | | |
| 20 | 95135_at | Cluster Incl AI844396 UI-M-AL1-ahp-e-05-0-UI s1 Mus musculus cDNA, 3 end/clone = UI-M-AL1-ahp-e-05-0-UI/ | 483.9 | P | 760.9 | P | 557.3 | P |
| | | clone_end = 3/gb = AI844396/gi = 5488302/ug = Mm 29429/len = 432/STRA = for | | | | | | |
| 20 | 95657_f_at | Cluster Incl AW125164 UI-M-BH2.1-apz-e-09-0-UI s1 Mus musculus cDNA, 3 end/clone = UI-M-BH2 1-apz-e-09-0-UI/ | 444.3 | P | 622.5 | P | 591.2 | P |
| | | clone_end = 3/gb = AW125164/gi = 6100694/ug = Mm 29541/len = 210/STRA = for | | | | | | |
| 20 | 95688_at | Cluster Incl Y08460: Degenerative spermatocyte homolog (Drosophila)/cds = (41,1012)/gb = Y08460/gi = 2225940/ | 1206 | P | 1329.4 | P | 1741.3 | P |
| | | ug = Mm 29648/len = 1778/STRA = for | | | | | | |
| 20 | 96245_at | Cluster Incl AW124530: UI-M-BH2 1-apo-g-09-0-UI s1 Mus musculus cDNA, 3 end/clone = UI-M-BH2 1-apo-g-09-0-UI/ | 219.2 | P | 542.8 | P | 414.5 | P |
| | | clone_end = 3/gb = AW124530/gi = 6100060/ug = Mm 29810/len = 487/STRA = for | | | | | | |
| 20 | 96252_at | Cluster Incl AJ005073 Mus musculus mRNA for Alix (ALG-2-Interacting protein X), complete CDS/cds = (0,2609)/ | 760.1 | P | 942.2 | P | 857.8 | P |
| | | gb = AJ005073/gi = 3550455/ug = Mm 29816/len = 2610/STRA = for | | | | | | |
| 20 | 96340_at | Cluster Incl AW124185 UI-M-BH2 1-aph-a-04-0-UI s1 Mus musculus cDNA, 3 end/clone = UI-M-BH2 1-aph-a-04-0-UI/ | 294.4 | P | 492.7 | P | 459.3 | P |
| | | clone_end = 3/gb = AW124185/gi = 6099715/ug = Mm 29987/len = 399/STRA = for | | | | | | |
| 20 | 97451_at | Cluster Incl AI837599 UI-M-AK0-adg-a-10-0-UI s1 Mus musculus cDNA, 3 end/clone = UI-M-AK0-adg-a-10-0-UI/ | 341.3 | P | 234.3 | P | 748.6 | P |
| | | clone_end = 3/gb = AI837599/gi = 5471812/ug = Mm 30251/len = 449/STRA = for | | | | | | |
| 20 | 97529_at | Cluster Incl AJ002390 Annexin 8/cds = (53,1036)/gb = AJ002390/gi = 2612794/ug = Mm 3267/len = 1888/STRA = for | 613.7 | P | 3504.3 | P | 2826.2 | P |
| 20 | 98114_at | Cluster Incl AF003348 Niemann Pick type C1/cds = (123,3959)/gb = AF003348/gi = 2251241/ug = Mm 3484/len = 5029/ | 366.2 | P | 489.2 | P | 464 | P |
| | | STRA = for | | | | | | |
| 20 | 98130_at | Cluster Incl U85089 Mus musculus thioredoxin mRNA, nuclear gene encoding mitochondrial protein, complete cds/ | 761.8 | P | 967.3 | P | 915.4 | P |
| | | cds = (51,551)/gb = U85089/gi = 1814410/ug = Mm 3533/len = 1182/STRA = for | | | | | | |
| 20 | 98153_at | Cluster Incl L20509 Chaperonin subunit 3 (gamma)/cds = (83,1717)/gb = L20509/gi = 347838/ug = Mm 3576/len = 1937/ | 711.7 | P | 871.8 | P | 885.8 | P |
| | | STRA = for | | | | | | |
| 20 | 98600_at | Cluster Incl U41341: Mus musculus endothelial monocyte-activating polypeptide I mRNA, complete cds/cds = (0,296)/ | 3283.7 | P | 4355.1 | P | 4013.8 | P |
| | | gb = U41341/gi = 1150723/ug = Mm.383/len = 331/STRA = for | | | | | | |
| 20 | 98608_at | Cluster Incl AI845886 UI-M-AO1-aeg-b-09-0-UI.s1 Mus musculus cDNA, 3 end/clone = UI-M-AO1-aeg-h-09-0-UI/ | 1011.1 | P | 1252.7 | P | 1278.9 | P |
| | | clone_end = 3/gb = AI845886/gi = 5489792/ug = Mm.3845/len = 447/STRA = rev | | | | | | |
| 20 | 98628_f_at | Cluster Incl AF003695: Hypoxia Inducible factor 1, alpha subunit/cds = (257,2767)/gb = AF003695/gi = 2197094/ | 1149.4 | P | 2129.6 | P | 1992.9 | P |
| | | ug = Mm 3879/len = 3973/STRA = for | | | | | | |
| 20 | 98629_f_at | Cluster Incl Y09085 Hypoxia inducible factor 1, alpha subunit/cds = (153,2585)/gb = Y09085/gi = 2113880/ug = Mm 3879/ | 1217.8 | P | 2021.8 | P | 1820.8 | P |
| | | len = 3774/STRA = for | | | | | | |
| 20 | 99087_at | Cluster Incl AW060179: UI-M-BH1-amu-d-09-0-UI s1 Mus musculus cDNA, 3 end/clone = UI-M-BH1-amu-d-09-0-UI/ | 664.9 | P | 919.7 | P | 795.5 | P |
| | | clone_end = 3/gb = AW060179/gi = 6007945/ug = Mm 38994/len = 244/STRA = rev | | | | | | |
| 20 | 99101_at | Cluster Incl AB012580 Mus musculus mRNA for elF3 p66, complete cds/cds = (96,1739)/gb = AB012580/gi = 2992163/ | 764.8 | P | 934.4 | P | 1259.9 | P |
| | | ug = Mm 3955/len = 1849/STRA = for | | | | | | |
| 20 | 99182_at | Cluster Incl AI846712: UI-M-AN1-afi-g-12-0-UI s1 Mus musculus cDNA, 3 end/clone = UI-M-AN1-afi-g-12-0-UI/ | 423.1 | P | 510.6 | P | 482.2 | P |
| | | clone_end = 3/gb = AI846712/gi = 5490618/ug = Mm 41801/len = 381/STRA = rev | | | | | | |
| 20 | 100051_at | Cluster Incl U17297 Erythrocyte protein band 7 2/cds = (90,944)/gb = U17297/gi = 972906/ug = Mm.4441/len = 2821/ | 1019.8 | P | 1554.3 | P | 1773.4 | P |
| | | STRA = for | | | | | | |
| 20 | 100123_f_at | Cluster Incl X15202: Mouse mRNA for fibronectin receptor beta-chain (VLA5-homolog)/cds = (0,2398)/gb = X15202/ | 3410.6 | P | 3741.4 | P | 3969.6 | P |
| | | gi = 50986/ug = Mm 4712/len = 3491/STRA = for | | | | | | |
| 20 | 100124_r_at | Cluster Incl X15202: Mouse mRNA for fibronectin receptor beta-chain (VLA5-homolog)/cds = (0,2398)/gb = X15202/ | 1151.1 | P | 1316.4 | P | 1289.9 | P |
| | | gi = 50986/ug = Mm 4712/len = 3491/STRA = for | | | | | | |
| 20 | 100127_at | Cluster Incl M35523 Cellular retinoic acid binding protein II/cds = (115,531)/gb = M35523/gi = 192705/ug = Mm 4757/len = 868/ | 131.9 | P | 332.1 | P | 257.6 | P |
| | | STRA = for | | | | | | |
| 20 | 100142_at | Cluster Incl X95480: DiGeorge syndrome gene c/cds = (133,1779)/gb = X95480/gi = 1550786/ug = Mm 4891/len = 2987/ | 199.5 | P | 293.1 | P | 374.3 | P |
| | | STRA = for | | | | | | |
| 20 | 100154_at | Cluster Incl AIB36367: UI-M-AP0-abi-b-11-0-UI s1 Mus musculus cDNA, 3 end/clone = UI-M-AP0-abi-b-11-0-UI/ | 227.1 | P | 522.8 | P | 715.7 | P |
| | | clone_end = 3/gb = AI836367/gi = 5470580/ug = Mm 4975/len = 332/STRA = for | | | | | | |
| 20 | 100535_at | Cluster Incl U76112 Mus musculus translation repressor NAT1 mRNA, complete cds/cds = (306,3026)/gb = U76112/ | 325.1 | P | 585.9 | P | 497.2 | P |
| | | gi = 1903415/ug = Mm 525/len = 3789/STRA = for | | | | | | |
| 20 | 100581_at | Cluster Incl U59807 Cystatin B/cds = (0,296)/gb = U59807/gi = 1439569/ug = Mm 6095/len = 297/STRA = for | 2782.8 | P | 3714.9 | P | 3998.4 | P |
| 20 | 100597_at | Cluster Incl AW049730: UI-M-BH1-ani-g-03-0-UI s1 Mus musculus cDNA, 3 end/clone = UI-M-BH1-ani-g-03-0-UI/ | 308.7 | P | 406.3 | P | 472.5 | P |
| | | clone_end = 3/gb = AW049730/gi = 5910259/ug = Mm 6375/len = 348/STRA = for | | | | | | |
| 20 | 100606_at | Cluster Incl M18070 Mouse (with short incubation period) prion protein (PRNP) gene, complete cds/cds = (10,774)/ | 2333.4 | P | 3525.7 | P | 2999.4 | P |
| | | gb = M18070/gi = 200528/ug = Mm 648/len = 775/STRA = for | | | | | | |
| 20 | 101025_f_at | Cluster Incl AJ005559 Small proline-rich protein 2A/cds = (0,251)/gb = AJ005559/gi = 3093357/ug = Mm 6853/len = 252/ | 1379.1 | P | 2219.9 | P | 3363.1 | P |
| | | STRA = for | | | | | | |
| 20 | 101482_at | Cluster Incl D85137 Mouse mRNA for PP1gamma, complete cds/cds = (107,1078)/gb = D85137/gi = 1332408/ug = Mm 7793/ | 1131.4 | P | 1259.5 | P | 1235.6 | P |
| | | len = 2200/STRA = for | | | | | | |
| 20 | 101487_f_at | Cluster Incl U47737 Mus musculus thymic shared antigen-1 (TSA-1) gene, complete cds/cds = (106,498)/gb = U47737/ | 1805.4 | P | 2217.6 | P | 3552.7 | P |
| | | gi = 1199649/ug = Mm.788/len = 1117/STRA = for | | | | | | |
| 20 | 101963_at | Cluster Incl X06086 Cathepsin L/cds = (68,1072)/gb = X06086/gi = 53046/ug = Mm 930/len = 1374/STRA = for | 883.6 | P | 1821.6 | P | 1727.2 | P |
| 20 | 101993_at | Cluster Incl X56304 Tenascin C/cds = (126,6185)/gb = X56304/gi = 54768/ug = Mm 980/len = 6831/STRA = for | 503.3 | P | 1805.2 | P | 2165.2 | P |
| 20 | 102052_at | Cluster Incl AA871791 vq41a10 r1 Mus musculus cDNA, 5 end/clone = IMAGE-1096794/clone_end = 5/gb = AA871791/ | 309.5 | P | 423.9 | P | 493.4 | P |
| | | gi = 2967236/ug = Mm.10297/len = 292/STRA = for | | | | | | |
| 20 | 102315_at | Cluster Incl AW124570 UI-M-BH2 1-apt-d-08-0-UI s1 Mus musculus cDNA, 3 end/clone = UI-M-BH2 1-apt-d-08-0-UI/ | 143.4 | P | 303 | P | 346.6 | P |
| | | clone_end = 3/gb = AW124570/gi = 6100100/ug = Mm 10665/len = 450/STRA = for | | | | | | |
| 20 | 102348_at | Cluster Incl AI551087 vx36f11.x1 Mus musculus cDNA, 3 end/clone = IMAGE-1277325/clone_end = 3/gb = AI551087/ | 917.9 | P | 1090.3 | P | 1129.4 | P |
| | | gi = 4483450/ug = Mm.11223/len = 446/STRA = rev | | | | | | |
| 20 | 103035_at | Cluster Incl U60020: Transporter 1, ABC (ATP binding cassette)/cds = (0,2174)/gb = U60020/gi = 1399904/ug = Mm 16122/ | 228.6 | P | 438.4 | P | 443.9 | P |
| | | len = 2175/STRA = for | | | | | | |
| 20 | 103059_at | Cluster Incl X93038 M musculus mRNA for MATB protein/cds = (87,353)/gb = X93038/gi = 1085067/ug = Mm 1662/len = 522/ | 2027.2 | P | 2840.4 | P | 2915.9 | P |
| | | STRA = rev | | | | | | |
| 20 | 103416_at | Cluster Incl AI844810 UI-M-AH1-agx-b-06-0-UI s1 Mus musculus cDNA, 3 end/clone = UI-M-AH1-agx-b-06-0-UI/ | 1266 | P | 1810.9 | P | 2110.5 | P |
| | | clone_end = 3/gb = AI844810/gi = 5488716/ug = Mm 18856/len = 520/STRA = for | | | | | | |
| 20 | 103589_at | Cluster Incl AF053235 Mus musculus keratin 16 mRNA, complete cds/cds = (12,1421)/gb = AF053235/gi = 3925354/ | 902.9 | P | 4075.7 | P | 4877.7 | P |
| | | ug = Mm.19889/len = 1580/STRA = for | | | | | | |
| 20 | 103877_at | Cluster Incl AW060485 UI-M-BH1-ann-a-09-0-UI s1 Mus musculus cDNA, 3 end/clone = UI-M-BH1-ann-a-09-0-UI/ | 235.7 | P | 301 | P | 398.1 | P |
| | | clone_end = 3/gb = AW060485/gi = 6008236/ug = Mm 21184/len = 263/STRA = for | | | | | | |
| 20 | 104074_at | Cluster Incl AW212109 uo82e01.x1 Mus musculus cDNA, 3 end/clone = IMAGE-2649048/clone_end = 3/gb = AW212109/ | 366.5 | P | 309.1 | P | 356.6 | P |
| | | gi = 6518110/ug = Mm 21687/len = 298/STRA = for | | | | | | |
| 20 | 104105_at | Cluster Incl AI854665 UI-U-BH0-akc-a-09-0-UI s1 Mus musculus cDNA, 3 end/clone = UI-M-BH0-akc-a-09-0-UI/ | 259.8 | P | 498.4 | P | 455.2 | P |
| | | clone_end = 3/gb = AI854665/gi = 5498571/ug = Mm 21892/len = 486/STRA = for | | | | | | |
| 20 | 104283_at | Cluster Incl AI037493 ub62a05 r1 Mus musculus cDNA, 5 end/clone = IMAGE-1382288/clone_end = 5/gb = AI037493 | 52.4 | P | 138.2 | P | 256.3 | P |
| | | gi = 3261184/ug = Mm 22252/len = 416/STRA = for | | | | | | |
| 20 | 104300_at | Cluster Incl AI117936 uc41g02 r1 Mus musculus cDNA, 5 end/clone = IMAGE-1400594/clone_end = 5/gb = AI117936/ | 1041.3 | P | 912 | P | 1193.6 | P |
| | | gi = 3518260/ug = Mm 22343/len = 182/STRA = for | | | | | | |
| 20 | 104369_at | Cluster Incl AB012033 Mus musculus gene for keratin 6 alpha/cds = (0,1661)/gb = AB012033/gi = 3882140/ug = Mm 22629/ | 672.3 | P | 3264 | P | 3372.3 | P |
| | | len = 2259/STRA = for | | | | | | |
| 20 | 104370_s_at | Cluster Incl K02108 Keratin complex 2, gene 6A/cds = (49,1710)/gb = K02108/gi = 198634/ug = Mm 22629/len = 2005/ | 383.9 | P | 4479.3 | P | 4111.7 | P |
| | | STRA = for | | | | | | |
| 20 | 104386_f_at | Clusler Incl AI843901: UI-M-AK1-aeu-g-04-0-UI s1 Mus musculus cDNA, 3 end/clone = UI-M-AK1-aeu-g-04-0-UI/ | 774.5 | P | 1267.1 | P | 1392 | P |
| | | clone_end = 3/gb = AI843901/gi = 5478114/ug = Mm 227/len = 300/STRA = for | | | | | | |
| 20 | 104579_r_at | Cluster Incl AI195392: ue72102.r1 Mus musculus cDNA, 5 end/clone = IMAGE-149666/clone_end = 5/gb = AI195392/ | 315.7 | P | 730.5 | P | 469.1 | P |
| | | gi = 3747998/ug = Mm.23961/len = 262/STRA = for | | | | | | |
| 20 | 104605_at | Cluster Incl AW047554 UI-M-BH1-ama-g-03-0-UI s1 Mus musculus cDNA, 3 end/clone = UI-M-BH1-ama-g-03-0-UI/ | 619.9 | P | 911.5 | P | 1132 | P |
| | | clone_end = 3/gb = AW047554/gi = 5908083/ug = Mm 24126/len = 312/STRA = for | | | | | | |
| 20 | 104628_at | Cluster Incl X61172.Mouse mRNA for alpha-mannosidase II/cds = (87,3539)/gb = X61172/gi = 49943/ug = Mm 2433/ | 538.7 | P | 614.2 | P | 657.6 | P |
| | | len = 6080/STRA = for | | | | | | |
| 20 | 104716_at | Cluster Incl X60367: Retinol binding protein 1, cellular/cds = UNKNOWN/gb = X60367/gi = 50547/ug = Mm 2450/len = 2633/ | 248.9 | P | 484.1 | P | 923.6 | P |
| | | STRA = for | | | | | | |
| 20 | 94367_at | Cluster Incl AI850362 UI-M-BG1-aig-b-06-0-UI s1 Mus musculus cDNA, 3 end/clone = UI-M-BG1-aig-b-06-0-UI/ | 572.6 | P | 861.9 | P | 695.5 | P |
| | | clone_end = 3/gb = AI850362/gi = 5494268/ug = Mm 25309/len = 494/STRA = for | | | | | | |
| 20 | 94391_at | Cluster Incl AW123650 UI-M-BH2 1-apl-g-08-0-U1.s1 Mus musculus cDNA, 3 end/clone = UI-M-BH2 1-apl-g-08-0-UI/ | 160.3 | P | 303.4 | P | 261 | P |
| | | clone_end = 3/gb = AW123650/gi = 6099180/ug = Mm 25652/len = 417/STRA = for | | | | | | |
| 20 | 94433_at | Cluster Incl AW060684 UI-M-BH1-anl-e-02-0-UI s1 Mus musculus cDNA, 3 end/clone = UI-M-BH1-anl-e-02-0-UI/ | 1254.1 | P | 1883.4 | P | 2602.4 | P |
| | | clone_end = 3/gb = AW060684/gi = 6008435/ug = Mm.26492/len = 383/STRA = for | | | | | | |
| 20 | 94928_at | Cluster Incl X87128 M musculus p75 TNF receptor DNA/cds = (67,133)/gb = X87128/gi = 809043/ug = Mm 2666/len = 134/ | 105.8 | P | 184.8 | P | 179.8 | P |
| | | STRA = for | | | | | | |
| 20 | 94991_at | Cluster Incl AW046661.UI-M-BH1-ald-g-02-0-UI s1 Mus musculus cDNA, 3 end/clone = U-M-BH1-ald-g-02-0-UI/ | 155.7 | P | 194.8 | P | 286.3 | P |
| | | clone_end = 3/gb = AW046661/gi = 5907190/ug = Mm 27313/len = 430/STRA = for | | | | | | |
| 20 | 95542_at | Cluster Incl AI835858.UI-M-AI0-aat-c-09-0-UI s1 Mus musculus cDNA, 3 end/clone = UI-M-AI0-aal-c-09-0-UI/clone_end = 3/ | 1913.3 | P | 2350.5 | P | 1834.8 | P |
| | | gb = AI835858/gi = 5470071/ug = Mm 27685/len = 469/STRA = rev | | | | | | |
| 20 | 96187_at | Cluster Incl AI837021 UI-M-AP0-abj-a-11-0-UI s2 Mus musculus cDNA, 3 end/clone = UI-M-AP0-abj-a-11-0-UI/ | 475 | P | 771.8 | P | 754.6 | P |
| | | clone_end = 3/gb = AI837021/gi = 5471234/ug = Mm 28488/len = 182/STRA = for | | | | | | |
| 20 | 97333_at | Cluster Incl AW121185 UI-M-BH2 3-aok-b-01-0-UI s1 Mus musculus cDNA, 3 end/clone = UI-M-BH2 3-aok-b-01-0-UI/ | 974.1 | P | 1295.3 | P | 1470 | P |
| | | clone_end = 3/gb = AW121185/gi = 6096518/ug = Mm 29547/len = 415/STRA = for | | | | | | |
| 20 | 97383_at | Cluster Incl AI848623 UI-M-AH1-agv-d-05-0-UI s1 Mus musculus cDNA, 3 end/clone = UI-M-AH1-agv-d-05-0-UI/ | 615 | P | 1326 | P | 2024.3 | P |
| | | clone_end = 3/gb = AI848623/gi = 5492569/ug = Mm 29762/len = 466/STRA = rev | | | | | | |
| 20 | 97419_at | Cluster Incl AW060956 UI-M-BH1-anv-g-02-0-UI s1 Mus musculus cDNA, 3 end/clone = UI-M-BH1-anv-g-02-0-UI/ | 155.3 | P | 257.3 | P | 286.7 | P |
| | | clone_end = 3/gb = AW060956/gi = 6008707/ug = Mm 29985/len = 493/STRA = for | | | | | | |
| 20 | 97930= f = at | Cluster Incl AF033620 CD151 antigen/cds = (82,843)/gb AF033620/gi = 3298592/ug = Mm 30246/len = 1618/STRA = for | 828.3 | P | 1163.8 | P | 1368.8 | P |
| 20 | 97932_f_at | Cluster Incl D89290 CD151 antigen/cds = (327,1088)/gb = D89290/gi = 2447006/ug = Mm 30246/len = 1819/STRA = for | 1266.5 | P | 1513 | P | 1665.9 | P |
| 20 | 97950_at | Cluster Incl X75129 Xanthine dehydrogenase/cds = (25,4032)/gb = X75129/gi = 473040/ug = Mm 3043/len = 4554/STRA = for | 194.9 | P | 361.9 | P | 444.7 | P |
| 20 | 98001_at | Cluster Incl U58203 Mus musculus Lsc (lsc) oncogene mRNA, complete cds/cds = (91,2850)/gb = U58203/gi = 1389755/ | 99.3 | P | 184.2 | P | 184.6 | P |
| | | ug = Mm 3181/len = 3200/STRA = for | | | | | | |
| 20 | 98402_at | Cluster Incl AI843799 UI-M-AN1-afc-e-09-0-UI s1 Mus musculus cDNA, 3 end/clone = UI-M-AN1-afc-e-09-0-UI/ | 526.9 | P | 1283 | P | 964.5 | P |
| | | clone_end = 3/gb = AI843799/gi = 5478012/ug = Mm 3350/len = 402/STRA = for | | | | | | |
| 20 | 98418_at | Cluster Incl U10115-Dishevelled, dsh homolog (Drosophila)/cds = (262,2349)/gb = U10115/gi = 497689/ug = Mm 3400/ | 414 | P | 618.6 | P | 642.1 | P |
| | | len = 3286/STRA = for | | | | | | |
| 20 | 98423_at | Cluster Incl M81445 Gap junction membrane channel protein beta 2/cds = (0,680)/gb = M81445/gi = 192844/ug = Mm 34118/ | 2589 | P | 3887.4 | P | 3707.6 | P |
| | | len = 681/STRA = for | | | | | | |
| 20 | 99057_at | Cluster Incl M12379 Mouse Thy-1 2 glycoprotein gene, complete cds/cds = (0,488)/gb = M12379/gi = 202040/ug = Mm 3951/ | 1297.8 | P | 1344.6 | P | 1773.8 | P |
| | | len = 489/STRA = for | | | | | | |
| 20 | 100428_at | Cluster Incl U43327: Laminin, gamma 2/cds = (39,3617)/gb = U43327/gi = 1151216/ug = Mm 4717/len = 5159/STRA = for | 1359.7 | P | 2120.2 | P | 2521.2 | P |
| 20 | 100445_f_at | Cluster Incl X91825 M musculus mRNA for SPRR1b protein/cds = (45,506)/gb = X91825/gi = 1213026/ug = Mm 4826/ | 641.7 | P | 6371.1 | P | 6591.1 | P |
| | | len = 688/STRA = for | | | | | | |
| 20 | 100472_at | Cluster Incl D10727: Mus musculus mRNA for NDPP-1 protein, complete cds/cds = (857,2026)/gb = D10727/gi = 220499/ | 405.6 | P | 643.1 | P | 616.9 | P |
| | | ug = Mm 4941/len = 4308/STRA = rev | | | | | | |
| 20 | 100499_at | Cluster Incl D29797 Mouse mRNA for syntaxin 3A, complete cds/cds = (103,972)/gb = D29797/gi = 924267/ug = Mm 5122/ | 236.9 | P | 252.9 | P | 241.8 | P |
| | | len = 1073/STRA = for | | | | | | |
| 20 | 100946_at | Cluster Incl AF109906 Mus musculus MHC class III region RD gene, partial cds, Bf, C2, G9A, NG22, G9, HSP70, HSP70, | 1087.5 | P | 1794.4 | P | 2276.7 | P |
| | | HSC701, and smRNP genes, complete cds, G7A gene, partial cds, and unknown genes/cds = (0,1928)/gb = AF109906/ | | | | | | |
| | | gi = 3986763/ug = Mm 6388/len = 1929/STRA = for | | | | | | |
| 20 | 102203_at | Cluster Incl M95545 Placental protein 11 related/cds = (9,1373)/gb = M95545/gi = 201907/ug = Mm.1001/len = 2407/ | 711.7 | P | 1090.8 | P | 946.5 | P |
| | | STRA = rev | | | | | | |
| 20 | 102248_f_at | Cluster Incl Y17138.Calcium/calmodulin-dependent serine protein kinase/cds = (492,3266)/gb = Y17138/gi = 3087817/ | 525 | P | 516.1 | P | 557.5 | P |
| | | ug = Mm.10735/len = 4393/STRA = for | | | | | | |
| 20 | 102255_at | Cluster Incl AB015978 Oncostatin receptor/cds = (779,3691)/gb = AB015978/gi = 3721859/ug = Mm 10760/len = 4026/ | 116 | P | 383.1 | P | 407.2 | P |
| | | STRA = for | | | | | | |
| 20 | 103217_at | Cluster Incl Y14041: Mus musculus mRNA for CASH alpha protein/cds = (74,1528)/gb = Y14041/gi = 2653419/ug = Mm.1993/ | 506.5 | P | 474 | P | 625.8 | P |
| | | len = 2770/STRA = for | | | | | | |
| 20 | 103305_at | Cluster Incl L04678 Integrin beta 4/cds = UNKNOWN/gb = L04678/gi = 192151/ug = Mm.21117/len = 5907/STRA = for | 197.3 | P | 693.4 | P | 631 | P |
| 20 | 103306_at | Cluster Incl AW259940: um80f09 y1 Mus musculus cDNA, 5 end/clone = IMAGE-2317673/clone_end = 5/gb = AW259940/ | 76.4 | P | 183.5 | P | 119.3 | P |
| | | gi = 6632921/ug = Mm 21134/len = 429/STRA = for | | | | | | |
| 20 | 103448_at | Cluster Incl M83218 Mus musculus intracellular calcium-binding protein (MRP8) mRNA, complete cds/cds = (17,286)/ | 175.7 | P | 2829.2 | P | 3757.6 | P |
| | | gb = M83218/gi = 199809/ug = Mm 21567/len = 343/STRA = for | | | | | | |
| 20 | 103723_at | Cluster Incl AA608387 vo42b10 r1 Mus musculus cDNA, 5 end/clone = IMAGE-1052539/clone_end = 5/gb = AA608387/ | 75.9 | P | 346 | P | 279 | P |
| | | gi = 2455274/ug = Mm 24208/len = 390/STRA = for | | | | | | |
| 20 | 103930_at | Cluster lncl AI413179 mc22e12.x1 Mus musculus cDNA, 3 end/clone = IMAGE-349294/clone_end = 3/gb = AI413179/ | 264.4 | P | 453.4 | P | 366.7 | P |
| | | gi = 4256683/ug = Mm 25117/len = 446/STRA = for | | | | | | |
| 20 | 104260_at | Cluster Incl AW123061: UI-M-BH2.1-apc-a-11-0-UI.s1 Mus musculus cDNA, 3 end/clone = UI-M-BH2.1-apc-a-11-0-UI/ | 103.8 | P | 289.9 | P | 248.8 | P |
| | | clone_end = 3/gb = AW123061/gi = 6098591/ug = Mm.28411/len = 288/STRA = for | | | | | | |
| 20 | 104406_at | Cluster Incl AI060798 ub44a06 r1 Mus musculus cDNA, 5 end/clone = IMAGE-1380582/clone_end = 5/gb = AI060798/ | 322.6 | P | 554.9 | P | 685.5 | P |
| | | gi = 3336221/ug = Mm 28768/len = 445/STRA = for | | | | | | |
| 20 | 104469_at | Cluster Incl M73748 Mouse OTS-8 mRNA, complete cds/cds = (166,780)/gb = M73748/gi = 200164/ug = Mm 2976/len = 1661/ | 190.1 | P | 618.7 | P | 508.9 | P |
| | | STRA = for | | | | | | |
| 20 | 92978_s_at | Cluster Incl X16490 Plasminogen activator inhibitor, type II/cds = (0, 1247)/gb = X16490/gi = 53589/ug = Mm.5019/len = 1905/ | 438 | P | 1100.7 | P | 983 | P |
| | | STRA = for | | | | | | |
| 20 | 93371_at | Cluster Incl U88064: Mus musculus basonuclin mRNA, complete cds/cds = (43,2928)/gb = U88064/gi = 2408218/ | 253.9 | P | 423.9 | P | 359 | P |
| | | ug = Mm.6110/len = 4441/STRA = for | | | | | | |
| 20 | 93424_at | Cluster Incl AI836915 UI-M-AJ0-aav-c-11-0-UI.s1 Mus musculus cDNA, 3 end/clone = UI-M-AJ0-aav-c-11-0-UI/ | 314.9 | P | 614.2 | P | 591.1 | P |
| | | clone_end = 3/gb = AI836915/gi = 5471128/ug = Mm 6478/len = 422/STRA = rev | | | | | | |
| 20 | 94121_at | Cluster Incl AJ005566 Small proline-rich protein 2H/cds(31,357)/gb = AJ005566/gi = 3093370/ug = Mm.10693/len = 728/ | 742.6 | P | 2972.7 | P | 3396.4 | P |
| | | STRA = rev | | | | | | |
| 20 | 94796_at | Cluster Incl AA710643 vt51d05 r1 Mus musculus cDNA, 5 end/clone = IMAGE-1166601/clone_end = 5/gb = AA710643/ | 1355.4 | P | 1781.2 | P | 1803 | P |
| | | gi = 2720561/ug = Mm.1800/len = 254/STRA = for | | | | | | |
| 20 | 100772_g_at | Cluster Incl AF068182 Mus musculus B cell linker protein BLNK mRNA, complete cds/cds = (132,1505)/gb = AF068182/ | 155.2 | P | 277.6 | P | 215.5 | P |
| | | gi = 3406752/ug = Mm.9749/len = 1719/STRA = for | | | | | | |
| 20 | 101717_at | Cluster Incl U26176 Somatostatin receptor 4/cds = (0,1154)/gb = U26176/gi = 833840/ug = Mm 57026/len = 1155/STRA = for | 287.4 | P | 603.4 | P | 652.1 | P |
| 20 | 99701_f_at | Cluster Incl AJ005560 Small proline-rich protein 2B/cds = (0, 296)/gb = AJ005560/gi = 3093359/ug = Mm 23796/len = 297/ | 1047.2 | P | 2046.9 | P | 3440.4 | P |
| | | STRA = for | | | | | | |
| 20 | 101621_at | Cluster Incl AF008595.Mus musculus lacrimal gland protein mRNA, complete cds/cds = (17,298)/gb = AF008595/ | 362.3 | P | 635.1 | P | 647.4 | P |
| | | gi = 2338287/ug = Mm 6205/len = 420/STRA = for | | | | | | |
| 20 | 102108_f_at | Cluster Incl AI505453 vr15c02.x1 Mus musculus cDNA, 3 end/clone = IMAGE-1120706/clone_end = 3/gb = AI505453/ | 457.4 | P | 842.5 | P | 878.4 | P |
| | | gi = 4403304/ug = Mm 42551/len = 260/STRA = rev | | | | | | |
| 20 | 92618_at | Cluster Incl AW125253 UI-M-BH2.1-aps-f-11-0-UI s1 Mus musculus cDNA, 3 end/clone = UI-M-BH2 1-aps-f-11-0-UI/ | 183.6 | P | 419.4 | P | 587.1 | P |
| | | clone_end = 3/gb = AW125253/gi = 6100783/ug = Mm.1106/len = 245/STRA = for | | | | | | |
| 20 | 93798_at | Cluster Incl AI839988 UI-M-AH0-acs-e-11-0-UI s1 Mus musculus cDNA, 3 end/clone = UI-M-AH0-acs-e-11-0-UI/ | 690.7 | A | 904.3 | P | 1063.8 | P |
| | | clone_end = 3/gb = AI839988/gi = 5474201/ug = Mm.2140/len = 352/STRA = for | | | | | | |
| 20 | 96957_at | Cluster Incl AB006463 Mus musculus mRNA for KIP, complete cds/cds = (26,601)/gb = AB006463/gi = 4185596/ | 126.8 | P | 494.6 | P | 477.8 | P |
| | | ug = Mm.30217/len = 687/STRA = for | | | | | | |
| 20 | 99081_at | Cluster Incl AF010254.Complement component 1 inhibitor/cds = (66, 1580)/gb = AF010254/gi = 3236325/ug = Mm 38888/ | 321.8 | A | 564.8 | P | 744.9 | P |
| | | len = 1775/STRA = for | | | | | | |
| 20 | 99159_at | Cluster Incl AI842675 UI-M-AO1-aeo-b-01-0-UI s1 Mus musculus cDNA, 3 end/clone = UI-M-AO1-aeo-b-01-0-UI/ | 166.9 | P | 469.9 | P | 450.1 | P |
| | | clone_end = 3/gb = AI842675/gi = 5476888/ug = Mm.41656/len = 280/STRA = for | | | | | | |
| 20 | 100610_at | Cluster Incl AF058298 Mus musculus calpain small subunit mRNA, complete cds/cds = (34,843)/gb = AF058298/ | 1006.8 | A | 1075.5 | P | 1306.9 | P |
| | | gi = 3511115/ug = Mm 6534/len = 1401/STRA = for | | | | | | |
| 20 | 101024_i_at | Cluster Incl AJ005559 Small proline-rich protein 2A/cds = (0,251)/gb = AJ005559/gi = 3093357/ug = Mm.6853/len = 252/ | 1062.6 | P | 1733.5 | P | 4302 | P |
| | | STRA = for | | | | | | |
| 20 | 95035_at | Cluster Incl AI467390. vd15c12 x1 Mus musculus cDNA, 3 end/clone = IMAGE-792598/clone_end = 3/gb = AI467390/ | 334.2 | P | 478.9 | P | 518.6 | P |
| | | gi = 4320727/ug = Mm.27546/len = 326/STRA = for | | | | | | |
| 20 | 96207_at | Cluster Incl AW046449 UI-M-BH1-akp-e-11-0-UI s1 Mus musculus cDNA, 3 end/clone = UI-M-BH1-akp-e-11-0-UI/ | 445.3 | P | 969.9 | P | 793.6 | P |
| | | clone_end = 3/gb = AW046449/gi = 5906978/ug = Mm 28645/len = 373/STRA = for | | | | | | |
| 20 | 96212_at | Cluster Incl AI853918 UI-M-BH0-aju-f-03-0-UI.s1 Mus musculus cDNA, 3 end/clone = UI-M-BH0-aju-f-03-0-UI/ | 241.7 | P | 313.7 | P | 317.9 | P |
| | | clone_end = 3/gb = AI853918/gi = 5497824/ug = Mm.28662/len = 352/STRA = for | | | | | | |
| 20 | 96738_at | Cluster Incl U41765: Mus musculus metalloprotease/disintegrin/cysteine rich protein precursor (MDC9) cDNA, complete cds/ | 175.4 | A | 355.5 | P | 213 | P |
| | | cds = (13,2550)/gb = U41765/gi = 1235675/ug = Mm.28908/len = 3850/STRA = for | | | | | | |
| 20 | 98018_at | Cluster Incl L39017 Protein C receptor, endothelial/cds = (55, 783)/gb = L39017/gi = 728480/ug = Mm 3243/len = 1358/ | 74.8 | P | 232 | P | 247.8 | P |
| | | STRA = for | | | | | | |
| 20 | 98427_s_at | Cluster Incl M57999.Nuclear factor of kappa light chain gene enhancer in B-cells 1, p105/cds = (291,3206)/gb = M57999/ | 185.1 | P | 306.5 | P | 318.2 | P |
| | | gi = 201931/ug = Mm.3420/len = 3892/STRA = for | | | | | | |
| 20 | 101902_at | Cluster Incl X17459 Recombining binding protein suppressor of hairless (Drosophila)/cds = (0,1580)/gb = X17459/gi = 52756/ | 152.1 | P | 169.3 | P | 163.7 | P |
| | | ug = Mm 89726/len = 3986/STRA = for | | | | | | |
| 20 | 103286_at | Cluster Incl AB012611 Mus musculus Hbp mRNA for Hrs binding Protein, complete cds/cds = (64,1635)/gb = AB012611/ | 204.5 | P | 199.5 | P | 260.7 | P |
| | | gi = 3721584/ug = Mm 20891/len = 1780/STRA = for | | | | | | |
| 20 | 103497_at | Cluster Incl AA592351 Vo27b01 r1 Mus musculus cDNA, 5 end/ctone-IMAGE-1051081/clone_end = 5/gb = AA592351/ | 70.5 | P | 91 | P | 102.3 | P |
| | | gi = 2406014/ug = Mm.22318/len = 131/STRA = for | | | | | | |
| 20 | 103991_at | Cluster Incl M61704: Mouse embryonic alkaline phosphatase gene, complete cds/cds = (0, 1589)/gb = M61704/gi = 192976/ | 148.4 | P | 281.3 | P | 143.9 | P |
| | | ug = Mm.26096/len = 1590/STRA = for | | | | | | |
| 20 | 103995_at | Cluster Incl AF065441: Mus musculus FGF binding protein 1 mRNA complete cds/cds = (0,755)/gb = AF065441/ | 248.1 | P | 381.9 | A | 333.3 | P |
| | | gi = 3153884/ug = Mm.2643/len = 756/STRA = for | | | | | | |
| 20 | 104184_at | Cluster Incl D16497: Natnuretic peptide precursor type B/cds = (78, 443)/gb = D16497/gi = 493659/ug = Mm 2740/len = 668/ | 58.7 | P | 167 | P | 111.2 | P |
| | | STRA = for | | | | | | |
| 20 | 92369_at | Cluster Incl M92420 Transforming growth factor alpha/cds = (5, 484)/gb = M92420/gi = 202155/ug = Mm 3992/len = 497/ | 88 | P | 176.8 | P | 178.9 | P |
| | | STRA = for | | | | | | |
| 20 | 92474_at | Cluster Incl AF083497.untilled/cds = (31, 3255)/gb = AF083497/gi = 3986489/ug = Mm.42128/len = 3924/STRA = for | 62.4 | P | 96.3 | P | 146.4 | P |
| 20 | 95984_at | Cluster Incl AI586160 vu95c12.x1 Mus musculus cDNA, 3 end/clone = IMAGE-1209910/clone_end = 3/gb = AI536160/ | 46.1 | P | 215.9 | P | 143.9 | P |
| | | gi = Mm.25035/len = 483/STRA = rev | | | | | | |
| 20 | 96997_at | Custer Incl Y19035-Mus musculus mRNA for amilonde-sensitive Na + channel (BLINaC gene)/cds = (28, 1515)/gb = / | 114.3 | P | 149 | P | 129.8 | P |
| | | gi = 5824009/ug = Mm 88839/len = 1656/STRA = for | | | | | | |
| 20 | 100723_f_at | Cluster Incl AJ005563: Small proline-rich protein 2E/cds = (0,230)/gb = AJ005563/gi = 3093364/ug = Mm.19358/len = 231/ | 1319.2 | P | 2385.6 | P | 1949.5 | P |
| | | STRA = for | | | | | | |
| 20 | 93272_at | Cluster Incl AF062076 Mus musculus palmitylated serine/threonine kinase (Psk) mRNA, complete cds/cds = (0,917)/ | 165.2 | P | 158.7 | A | 316.2 | P |
| | | gb = AF062076/gi = 4106341/ug = Mm.17461/len = 918/STRA = for | | | | | | |
| 20 | 94224_s_at | Cluster Incl M74123 Mus musculus (strain C57BI/6) mRNA sequence/cds = UNKNOWN/gb = M74123/gi = 198867/ | −9.6 | A | 327.2 | P | 469.1 | P |
| | | ug = Mm 22262/len = 1623/STRA = for | | | | | | |
| 20 | 94264_at | Cluster Incl AW122170 UI-M-BH2 2-aor-g-12-0-UI s1 Mus musculus cDNA, 3 end/clone = UI-M-BH2 2-aor-g-12-0-UI/ | 605.4 | P | 690.7 | P | 730.5 | P |
| | | clone_end = 3/gb = AW122170/gi = 6097613/ug = Mm 22467/len = 188/STRA = for | | | | | | |
| 20 | 94918_at | Cluster Incl AI839392 UI-M-AN0-aco-b-02-0-UI.s1 Mus musculus cDNA, 3 end/clone = UI-M-AN0-aco-b-02-0-UI/ | 267.3 | A | 386.8 | P | 312.1 | P |
| | | clone_end = 3/gb = AI839392/gi = 5473605/ug = Mm 24174/len = 181/STRA = for | | | | | | |
| 20 | 96925_at | Cluster Incl AW122429 UI-M-BH2.2-aow-g-07-0-UI.s1 Mus musculus cDNA, 3 end/clone = UI-M-BH2 2-aow-g-07-0-UI/ | 4.9 | P | 76 | P | 74.6 | P |
| | | clone_end = 3/gb = AW122429/gi = 6097940/ug = Mm.30161/len = 477/STRA = for | | | | | | |
| 20 | 101551_s_at | Cluster Incl X78989 M. musculus mRNA for testin/cds = (120,1226)/gb = X78989/gi = 475207/ug = Mm.88645/len = 1377/ | 160 | P | 114.7 | A | 207.7 | P |
| | | STRA = for | | | | | | |
| 20 | 94380_at | Cluster Incl AI852581 UI-M-BH0-aiu-a-11-0-UI s1 Mus musculus cDNA, 3 end/clone = UI-M-BH0-aiu-a-11-0-UI/ | 378.8 | A | 641.6 | P | 720.5 | P |
| | | clone_end = 3/gb = AI852581/gi = 5496487/ug = Mm 25576/len = 399/STRA = for | | | | | | |
| 20 | 95591_at | Cluster Incl AI843335 UI-M-AQ1-adz-b-02-0-UI s1 Mus musculus cDNA, 3 end/clone = UI-M-AQ1-adz-b-02-0-UI/ | 273.4 | P | 498.6 | P | 515.3 | P |
| | | clone_end = 3/gb = AI843335/gi = 5477548/ug = Mm.27895/len = 197/STRA = for | | | | | | |
| 20 | 95599_at | Cluster Incl D28941 Mouse mRNA for alpha-2,3-sialyltransferase, complete cds/cds = (128,1129)/gb = D28941/gi = 469215/ | 93.5 | P | 206.4 | P | 300.8 | P |
| | | ug = Mm 2793/len = 1919/STRA = for | | | | | | |
| 20 | 96208_at | Cluster Incl AW123514.UI-M-BH2 1-aqb-c-04-0-UI s1 Mus musculus cDNA, 3 end/clone = UI-M-BH2 1-aqb-c-04-0-UI/ | 159.6 | P | 191.7 | A | 229.3 | P |
| | | clone_end = 3/gb = AW123514/gi = 6099044/ug = Mm 28646/len = 374/STRA = for | | | | | | |
| 20 | 96824_at | Cluster Incl AB025354: Mus musculus genes for Supl15h and Sox15.partial cds/cds = (0,168)/gb = AB025354/gi = 5103143/ | 291.3 | P | 738.3 | P | 603.2 | P |
| | | ug = Mm 29387/len = 169/STRA = for | | | | | | |
| 20 | 97354_at | Cluster Incl AI836082: UI-M-AJ0-abb-f-05-0-UI s1 Mus musculus cDNA, 3 end/clone = UI-M-AJ0-abb-f-05-0-UI/ | 197.8 | P | 329.4 | P | 376.9 | P |
| | | clone_end = 3/gb = AI836082/gi = 5470295/ug = Mm 29630/len = 432/STRA = rev | | | | | | |
| 20 | 99489_at | Cluster Incl U23921 Mus musculus osmotic stress protein 94 (Osp94) mRNA, complete cds/cds = (182,2698)/gb = U23921/ | 85.9 | P | 318.5 | P | 253.6 | P |
| | | gi = 1098540/ug = Mm 4150/len = 2898/STRA = for | | | | | | |
| 20 | 99952_at | Cluster Incl X93035 Glycoprotein 39/cds = (65,1210)/gb = X93035/gi = 1085065/ug = Mm 4376/len = 1616/STRA = rev | 58.3 | P | 1244.3 | P | 703.1 | P |
| 20 | 100882_at | Cluster Incl AF003525 Defensin beta 1/cds = (42,251)/gb = AF003525/gi = 2197074/ug = Mm.5341/len = 398/STRA = for | 170.8 | P | 590.5 | P | 454.1 | P |
| 20 | 103222_at | Cluster Incl L21671 Mouse Eps8 mRNA sequence, complete cds/cds = (245,2710)/gb = L21671/gi = 309216/ug = Mm 2012/ | 49.9 | P | 69.1 | A | 84.5 | P |
| | | len = 3550/STRA = rev | | | | | | |
| 20 | 104173_at | Cluster Incl AA797989 ub60d04.r1 Mus musculus cDNA, 5 end/clone = IMAGE-1382119/clone_end = 5/gb = AA797989/ | 495.3 | M | 386.6 | P | 483.4 | P |
| | | gi = 2860944/ug = Mm 27253/len = 413/STRA = for | | | | | | |
| 20 | 104211_at | Cluster Incl D13867 Mouse mRNA for VLA-3 alpha subunit/cds = (0, 3161)/gb = D13867/gi = 220634/ug = Mm 2775/len = 3162/ | 203.4 | P | 525.6 | P | 477.4 | P |
| | | STRA = for | | | | | | |
| 20 | 92979_at | Cluster Incl X63190 Polyomavirus enhancer activator 3/cds = (125, 1792)/gb = X63190/gi = 53627/ug = Mm 5025/len = 2410/ | 222.6 | P | 326.7 | P | 298.3 | P |
| | | STRA = for | | | | | | |
| 20 | 93672_at | Cluster Incl U09928 Protein kinase, interferon inducible double stranded RNA dependent/cds = (16,1563)/gb = U09928/ | 83.1 | P | 179.2 | P | 173 | P |
| | | gi = 3273413/ug = Mm 807/len = 2191/STRA = for | | | | | | |
| 20 | 93914_at | Cluster Incl M20658 Interleukin 1 receptor, type 1/cds = (224,1954)/gb = M20658/gi = 198300/ug = Mm 896/len = 4740/ | 248.3 | P | 348.5 | P | 357 | P |
| | | STRA = for | | | | | | |
| 20 | 94753_at | Clusler Incl M80632 Guanine nucleotide binding protein, alpha 15/cds = (42,1166)/gb = M80632/gi = 193570/ug = Mm 1546/ | −35.1 | P | 240.1 | P | 216.5 | A |
| | | len = 1353/STRA = for | | | | | | |
| 20 | 100353_g_at | Cluster Incl AA919208 vy50h03 r1 Mus musculus cDNA, 5 end/clone = IMAGE-1298933/clone_end = 5/gb = AA919208/ | 148.3 | P | 345.4 | P | 374.8 | P |
| | | gi = 3065987/ug = Mm 88724/len = 501/STRA = for | | | | | | |
| 20 | 95794_f_at | Cluster Incl AJ005567 Small proline-rich protein 21/cds = (0,230)/gb = AJ005567/gi = 3093372/ug = Mm 87821/len = 231/ | 570.8 | P | 1459.9 | P | 1505 | P |
| | | STRA = for | | | | | | |
| 20 | 93602_at | Cluster Incl AF074714 Mus musculus milogen-and stress-activated protein kinase-2 (mMSK2) mRNA, complete cds/ | 342.8 | A | 1200.4 | P | 1053.5 | P |
| | | cds = (52,2373)/gb = AF074714/gi = 3786405/ug = Mm 20914/len = 3120/STRA = for | | | | | | |
| 20 | 96259_at | Cluster Incl U35646 Puromycin-sensitive aminopeptidase/cds = (105,2867)/gb = U35646/gi = 1184160/ug = Mm.29824/ | 442.5 | A | 623.5 | P | 589.7 | P |
| | | len = 3051/STRA = for | | | | | | |
| 20 | 100621_at | Cluster Incl AI848825 UI-M-AJ1-ahb-b-03-0-UI s1 Mus musculus cDNA, 3 end/clone = UI-M-AJ1-ahb-b-03-0-UI/ | 340.3 | P | 699.2 | P | 1421.5 | P |
| | | clone_end = 3/gb = AI848825/gi = 5492731/ug = Mm.6588/len = 342/STRA = for | | | | | | |
| 20 | 104136_at | Cluster Incl AI840413 UI-M-AH0-acv-h-08-0-UI.s1 Mus musculus cDNA, 3 end/clone = UI-M-AH0-acv-h-08-0-UI/ | 199.7 | P | 256 | P | 267.8 | A |
| | | clone_end = 3/gb = AI840413/gi = 5474626/ug = Mm.22094/len = 266/STRA = rev | | | | | | |
| 20 | 104374_at | Cluster Incl M64086 Mouse spi2 proteinase inhibitor (spi2/eb4) mRNA, complete cds/cds = (60,1316)/gb = M64086/ | −14.7 | M | 1864.8 | P | 1644.9 | P |
| | | gi = 201039/ug = Mm.22650/len = 2035/STRA = rev | | | | | | |
| 20 | 104737_at | Cluster Incl AA689670 vs03b05.r1 Mus musculus cDNA, 5 end/clone = IMAGE-1137105/clone_end = 5/gb = AA669670/ | 364.9 | P | 977.1 | P | 1388.2 | P |
| | | gi = 2690606/ug = Mm.24660/len = 238/STRA = for | | | | | | |
| 20 | 95034_f_at | Cluster Incl AW212243 uo84d09 x1 Mus musculus cDNA, 3 end/clone = IMAGE-2649233/clone_end = 3/gb = AW212243/ | 205.2 | P | 284.1 | P | 253.8 | P |
| | | gi = 6518272/ug = Mm 27546/len = 250/STRA = for | | | | | | |
| 20 | 95618_at | Clusler Incl AI843884 UI-M-AK1-aeu-e-10-0-UI s1 Mus musculus cDNA, 3 end/clone = UI-M-AK1-aeu-e-10-0-UI/ | 185 | A | 214.7 | P | 210.2 | P |
| | | clone_end = 3/gb = AI843884/gi = 5478097/ug = Mm.28032/len = 392/STRA = rev | | | | | | |
| 20 | 103238_at | Cluster Incl M89797 Wingless-related MMTV integration site 4/cds = (45,1100)/gb = M89797/gi = 202401/ug = Mm 20355/ | 268 | P | 337.5 | A | 497.8 | P |
| | | len = 1101/STRA = for | | | | | | |
| 20 | 103445_at | Cluster Incl M18401 Mus musculus putative homeobox 2.2 protein gene, partial cds/cds = (0,293)/gb = M18401/gi = 193938/ | 271.1 | A | 375.4 | P | 305.7 | P |
| | | ug = Mm.215/len = 294/STRA = for | | | | | | |
| 20 | 104198_at | Cluster Incl AW211760 uo85c06 y1 Mus musculus cDNA, 5 end/clone = IMAGE-2649322/clone_end = 5/gb = AW211760/ | 371.3 | P | 540.7 | P | 474.8 | P |
| | | gi = 6517732/ug = Mm 27519/len = 462/STRA = for | | | | | | |
| 20 | 104210_at | Cluster Incl AI839082 UI-M-AK0-adl-f-03-0-UI s1 Mus musculus cDNA, 3 end/clone = UI-M-AK0-adl-f-03-0-UI/clone_end = 3/ | 0.2 | P | 229.3 | P | 227.1 | P |
| | | gb = AI839082/gi = 5473295/ug = Mm.2775/len = 212/STRA = for | | | | | | |
| 20 | 92276_at | Cluster Incl AB021861: Mus musculus mRNA for apoptosis signal-regulating kinase 2, complete cds/cds = (269,4138)/ | 179.5 | A | 361.9 | P | 374.3 | P |
| | | gb = AB021861/gi = 4996560/ug = Mm.36640/len = 4325/STRA = for | | | | | | |
| 20 | 92280_at | Cluster Incl AA867778 vx16e04 r1 Mus musculus cDNA, 5 end/clone = IMAGE-1264638/clone_end = 5/gb = AA867778/ | 204.1 | A | 222.3 | P | 276.4 | P |
| | | gi = 2963223/ug = Mm 3678/len = 512/STRA = for | | | | | | |
| 20 | 92759_at | Cluster Incl U43298 Laminin, beta 3/cds = (156,3662)/gb = U43298/gi = 1151214/ug = Mm 4732/len = 4049/STRA = for | 235.1 | P | 511.5 | P | 595.7 | P |
| 20 | 94791_s_at | Cluster Incl M90470 Leukocyte tyrosine kinase/cds = (137,2803)/gb = M90470/gi = 198885/ug = Mm.1740/len = 3077/ | 601.8 | A | 1014.9 | P | 1007.1 | P |
| | | STRA = for | | | | | | |
| 20 | 96566_at | Cluster Incl L10385.Mus musculus transglutaminase E (TGASE3) mRNA, complete cds/cds = (54,2135)/gb = L10385/ | 298.4 | P | 379.4 | A | 308 | P |
| | | gi = 309520/ug = Mm 2961/len = 2297/STRA = for | | | | | | |
| 20 | 98372_at | Cluster Incl AW050387 UI-M-BH1-ang-h-10-0-UI s1 Mus musculus cDNA, 3 end/clone = UI-M-BH1-ang-h-10-0-UI/ | 113.2 | A | 149.9 | P | 138.9 | P |
| | | clone_end = 3/gb = AW050387/gi = 5910916/ug = Mm.4458/len = 380/STRA = for | | | | | | |
| 20 | 95793_at | Cluster Incl AJ005562 Small proline-rich protein 2D/cds = (43,300)/gb = AJ005562/gi = 3093362/ug = Mm 87820/len = 668/ | 332.8 | P | 2875.6 | P | 2199 | P |
| | | STRA = for | | | | | | |
| 20 | 92870_at | Cluster Incl AF063095.Mus musculus SEL1L (Sel1l) mRNA, complete cds/cds = (75,2297)/gb = AF063095/gi = 4159994/ | 151 | A | 244.3 | P | 184.9 | P |
| | | ug = Mm 14352/len = 3663/STRA = for | | | | | | |
| 20 | 93105_s_at | Cluster Incl M26056 Mouse T-cell receptor germline beta-chain gene constant region (CT)/cds = (0,520)/gb = M26056/ | 313.4 | P | 363.1 | P | 401.7 | P |
| | | gi = 201239/ug = Mm 16654/len = 521/STRA = for | | | | | | |
| 20 | 93795_at | Cluster Incl AW124626 UI-M-BH2 1-apw-b-07-0-UI.s1 Mus musculus cDNA, 3 end/clone = UI-M-BH2 1-apw-b-07-0-UI/ | 171.3 | M | 304 | M | 297.5 | P |
| | | clone_end = 3/gb = AW124626/gi = 6100156/ug = Mm 21399/len = 416/STRA = for | | | | | | |
| 20 | 94260_at | Cluster Incl AI850352 UI-M-BG1-aig-a-08-0-UI s1 Mus musculus cDNA, 3 end/clone = UI-M-BG1-aig-a-08-0-UI/ | 297 | M | 820.5 | P | 565.3 | P |
| | | clone_end = 3/gb = AI850352/gi = 5494258/ug = Mm 22428/len = 420/STRA = for | | | | | | |
| 20 | 94316_at | Cluster Incl AB012042 Mus musculus gene for keratin 6 beta/cds = (287,1975)/gb = AB012042/gi = 3845567/ug = Mm 22657/ | 303.5 | M | 3374.4 | P | 2382.2 | A |
| | | len = 2299/STRA = for | | | | | | |
| 20 | 104601_at | Cluster Incl X14432 Thrombomodulin/cds = (158,1891)/gb = X14432/gi = 54781/ug = Mm 24096/len = 3636/STRA = for | 78 | A | 227.3 | P | 215.4 | P |
| 20 | 97420_at | Cluster Incl AW230891 uo69b04.y1 Mus musculus cDNA, 5 end/clone = IMAGE-2647759/clone_end = 5/gb = AW230891/ | 0.8 | A | 571 | P | 512.3 | P |
| | | gi = 6560187/ug = Mm 29986/len = 523/STRA = rev | | | | | | |
| 20 | 100884_at | Cluster Incl U04204 Fibroblast growth factor regulated protein/cds = (67,1017)/gb = U04204/gi = 463376/ug = Mm 5378/ | 29.9 | M | 466.8 | P | 484.3 | P |
| | | len = 1304/STRA = for | | | | | | |
| 20 | 101464_at | Cluster Incl V00755 Interferon beta, fibroblast/cds = (0,118)/gb = V00755/gi = 51554/ug = Mm 8245/len = 222/STRA = for | 1243.7 | A | 1699.9 | P | 1984.7 | P |
| 20 | 102889_r_at | Cluster Incl X86569 LIM-domain containing, protein kinase/cds = (322,2265)/gb = X86569/gi = 1051159/ug = Mm.15409/ | 51.2 | A | 348.4 | P | 307 | P |
| | | len = 3439/STRA = for | | | | | | |
| 20 | 93000_g_at | Cluster Incl D50416 Mouse mRNA for AREC3, complete cds/cds = (61,2388)/gb = D50416/gi = 1255625/ug = Mm.5085/ | 93 | A | 127.3 | P | 173.8 | P |
| | | len = 5566/STRA = for | | | | | | |
| 20 | 93204_r_at | Cluster Incl AW107463 um17a12 x1 Mus musculus cDNA, 3 end/clone = IMAGE-2192542/clone_end = 3/gb = AW107463/ | 6.8 | A | 64.3 | P | 42.6 | P |
| | | gi = 6078263/ug = Mm.5697/len = 484/STRA = rev | | | | | | |
| 20 | 97790_s_at | Cluster Incl X84014 Laminin, alpha 3/cds = (0,7710)/gb = X84014/gi = 1922890/ug = Mm.42012/len = 8077/STRA = for | −86.9 | P | 210.5 | P | 279.8 | P |
| 20 | 100333_at | Cluster Incl M13521 Murine serum amyloid A-1 (SAA-1) gene/cds = (0,368)/gb = M13521/gi = 200905/ug = Mm 87850/ | 685.6 | A | 897.4 | P | 947.6 | P |
| | | len = 369/STRA = for | | | | | | |
| 20 | 92858_at | Cluster Incl AF002719 Mus musculus secretory leukoprotease inhibitor gene, complete cds/cds = (20,415)/gb = AF002719/ | −11.9 | A | 1089.4 | P | 1087.9 | P |
| | | gi = 4100898/ug = Mm 1395/len = 660/STRA = for | | | | | | |
| 20 | 93603_at | Cluster Incl AF034092 Mus musculus nuclear localization signal containing protein (Nlvcf) mRNA, complete cds/ | 145.8 | A | 168.5 | A | 202.9 | P |
| | | cds = (114,734)/gb = AF034092/gi = 3834385/ug = Mm.20918/len = 874/STRA = for | | | | | | |
| 20 | 96093_at | Cluster Incl AI842705 UI-M-AO1-aeo-d-09-0-UI s1 Mus musculus cDNA, 3 end/clone = UI-M-AO1-aeo-d-09-0-UI/ | 385.4 | A | 402.3 | A | 402.5 | A |
| | | clone_end = 3/gb = AI842705/gi = 5476918/ug = Mm.26740/len = 300/STRA = for | | | | | | |
| 20 | 98892_at | Cluster Incl AI846934 UI-M-AK1-aey-e-06-0-UI s1 Mus musculus cDNA, 3 end/clone = UI-M-AK1-aey-e-06-0-UI/ | 49.6 | A | 80.8 | M | 132 | P |
| | | clone_end = 3/gb = AI846934/gi = 5490840/ug = Mm.28548/len = 407/STRA = for | | | | | | |
| 20 | 104730_at | Cluster Incl AW046889: UI-M-BH1-akl-d-04-0-UI s1 Mus musculus cDNA, 3 end/clone = UI-M-BH1-akl-d-04-0-UI/ | 6.7 | A | 119.8 | A | 238.7 | A |
| | | clone_end = 3/gb = AW046889/gi = 5907418/ug = Mm 24627/len = 369/STRA = rev | | | | | | |
| 20 | 94383_at | Cluster Incl D50586 Tissue factor pathway inhibitor 2/cds = (35,727)/gb = D50586/gi = 2467368/ug = Mm 25612/len = 1522/ | 8 | A | 96.8 | P | 125.7 | P |
| | | STRA = for | | | | | | |
| 20 | 94924_at | Cluster Incl AW122679 UI-M-BH2 2-aol-h-11-0-UI s1 Mus musculus cDNA, 3 end/clone = UI-M-BH2.2-aol-h-11-0-UI/ | 627.5 | A | 866.9 | M | 860.6 | P |
| | | clone_end = 3/gb = AW122679/gi = 6098209/ug = Mm 2664/len = 291/STRA = for | | | | | | |
| 20 | 96764_at | Cluster Incl AJ007971 Mus musculus mRNA for IIGP protein/cds = (104,1345)/gb = AJ007971/gi = 4158173/ug = Mm 29008/ | 6.4 | A | 258.3 | P | 725.3 | P |
| | | len = 2280/STRA = for | | | | | | |
| 20 | 100976_at | Cluster Incl AF013490 Mus musculus protein-tyrosine phosphatase mRNA, complete cds/cds = (96,1877)/gb = AF013490/ | 159.4 | P | 536.1 | M | 447.2 | P |
| | | gi = 2331298/ug = Mm.6678/len = 3531/STRA = for | | | | | | |
| 20 | 101384_at | Cluster Incl AI852933 UI-M-BH0-aiz-c-09-0-UI s1 Mus musculus cDNA, 3 end/clone = UI-M-BH0-aiz-c-09-0-UI/ | 266.3 | P | 420.8 | P | 827.7 | P |
| | | clone_end = 3/gb = AI852933/gi = 5496839/ug = Mm.7114/len = 395/STRA = for | | | | | | |
| 20 | 102689_at | Cluster Incl AF110520 Mus musculus major histocompatibility complex region NG27, NG28, RPS28, NADH | 301.4 | A | 486.5 | P | 414.5 | P |
| | | oxidoreductase, NG29, KIFC1, Fas-binding protein, BING1, tapasin, RalGDS-like, KE2, BING4, beta 1,3-galactosyl | | | | | | |
| | | transferase, and RPS18 genes, complete cds, S/STRA = for | | | | | | |
| 20 | 102712_at | Cluster Incl X03505 Serum amyloid A 3/cds = (0,368)/gb = X03505/gi = 54028/ug = Mm 14277/len = 369/STRA = for | −35.5 | M | 1048.6 | P | 2802 | P |
| 20 | 104035_at | Cluster Incl AW123795 UI-M-BH2.1-apm-e-02-0-UI s1 Mus musculus cDNA, 3 end/clone = UI-M-BH2 1-apm-e-02-0-UI/ | 132.9 | P | 279.7 | P | 161.7 | P |
| | | clone_end = 3/gb = AW123795/gi = 6099325/ug = Mm 27057/len = 404/STRA = rev | | | | | | |
| 20 | 93145_at | Cluster Incl AI840824 UI-M-AH0-adb-d-03-0-UI s1 Mus musculus cDNA, 3 end/clone = UI-M-AH0-adb-d-03-0-UI/ | 70.7 | A | 96.5 | P | 108.6 | P |
| | | clone_end = 3/gb = AI840824/gi = 5475037/ug = Mm 5209/len = 447/STRA = for | | | | | | |
| 20 | 93878_at | Cluster Incl AF010135 Mus musculus maf10 (mAF10) mRNA, complete cds/cds = (178,3384)/gb = AF010135/gi = 2809380/ | 204.3 | A | 243.7 | P | 238.4 | A |
| | | ug = Mm 8866/len = 3543/STRA = for | | | | | | |
| 20 | 100308_at | Cluster Incl X66976.M. musculus col8a1 gene, exon 1 (and joined CDS)/cds = (0,2231)/gb = X66976/gi = 50493/ | 63.8 | M | 168.1 | A | 223.1 | P |
| | | ug = Mm 86813/len = 2232/STRA = for | | | | | | |
| 20 | 97598_at | Cluster Incl AW209139: uo02d07.x1 Mus musculus cDNA, 3 end/clone = IMAGE-2582221/clone_end = 3/gb = AW209139/ | 22.6 | A | 61.2 | P | 93.5 | A |
| | | gi = 6515079/ug = Mm.89650/len = 425/STRA = rev | | | | | | |
| 20 | 102423_at | Cluster Incl AI649125 uk31e12 x1 Mus musculus cDNA, 3 end/clone = IMAGE-1970638/clone_end = 3/gb = AI649125/ | 16.7 | A | 74.9 | P | 50.2 | P |
| | | gi = 4729959/ug = Mm.12790/len = 593/STRA = for | | | | | | |
| 20 | 102763_at | Cluster Incl AF064748 Mus musculus S3-12 mRNA, complete cds/cds = (138,4349)/gb = AF064748/gi = 3236367/ | −43.3 | A | 126.6 | A | 106.9 | A |
| | | ug = Mm 12966/len = 5774/STRA = for | | | | | | |
| 20 | 103080_at | Cluster Incl U15635 Mus musculus IFN-gamma induced (Mg11) mRNA, complete cds/cds = (57,1841)/gb = U15635/ | 78.2 | A | 267.2 | P | 221.5 | P |
| | | gi = 558902/ug = Mm.17167/len = 1942/STRA = for | | | | | | |
| 20 | 103634_at | Cluster Incl U51992.lnterferon dependent positive acting transcription factor 3 gamma/cds = (64,1263)/gb = U51992/ | 77.9 | A | 315.3 | P | 492.6 | P |
| | | gi = 1263309/ug = Mm 2032/len = 1959/STRA = for | | | | | | |
| 20 | 104293_at | Cluster Incl AI882440 uc01f05 r1 Mus musculus cDNA, 5 end/clone = IMAGE-1396737/clone_end = 5/gb = AI882440/ | −192.1 | A | 95.1 | A | 86.1 | P |
| | | gi = 5567529/ug = Mm 22306/len = 271/STRA = for | | | | | | |
| 20 | 96123_at | Cluster Incl X99347 M musculus mRNA for LPS-binding protein/cds = (127,1572)/gb = X99347/gi = 1430866/ug = Mm 28110/ | 13.1 | A | 423.4 | P | 268.4 | P |
| | | len = 1925/STRA = for | | | | | | |
| 20 | 96766_s_at | Cluster Incl AB000828 Mouse mRNA for receptor tyrosine kinase, complete cds/cds = (13,2655)/gb = AB000828/ | 92.9 | A | 117.7 | A | 345.3 | P |
| | | gi = 1871412/ug = Mm 2901/len = 2845/STRA = for | | | | | | |
| 20 | 97971_at | Cluster Incl AI852148 UI-M-BH0-ajb-d-12-0-UI s1 Mus musculus cDNA, 3 end/clone = UI-M-BH0-ajb-d-12-0-UI/ | 193.2 | A | 232 | P | 235.1 | P |
| | | clone_end = 3/gb = AI852148/gi = 5496049/ug = Mm 30991/len = 375/STRA = for | | | | | | |
| 20 | 98501_at | Cluster Incl Y07519 Lymphocyte antigen 84/cds = (194,1207)/gb = Y07519/gi = 55517/ug = Mm 35692/len = 2646/STRA = for | −12 | A | 47.8 | P | 213.2 | P |
| 20 | 100484_at | Cluster Incl X66473 Matrix metalloproteinase 1/cds = (10,1428)/gb = X66473/gi = 53603/ug = Mm 5022/len = 2660/STRA = for | 36.7 | A | 118.3 | P | 228.4 | P |
| 20 | 100983_at | Cluster Incl AB022053 Mus musculus gene for prolyl oligopeptidase/cds = (377,2509)/gb = AB022053/gi = 5689034/ | 183.1 | M | 273.3 | P | 269.4 | A |
| | | ug = Mm 6735/len = 2981/STRA = for | | | | | | |
| 20 | 102637_at | Cluster Incl AF039601 Mus musculus betaglycan mRNA, complete cds/cds = (231,2783)/gb = AF039601/gi = 3387819/ | 58.5 | A | 124.1 | P | 118.1 | P |
| | | ug = Mm 12960/len = 3977/STRA = rev | | | | | | |
| 20 | 103446_at | Cluster Incl AA959954 vw53a09 s1 Mus musculus cDNA, 3 end/clone = IMAGE-1247512/clone_end = 3/gb = AA959954/ | −157.5 | A | 27 | A | 163.2 | P |
| | | gi = 3125854/ug = Mm 21551/len = 524/STRA = for | | | | | | |
| 20 | 92234_at | Cluster Incl X66223 M musculus mRNA for retinoid X receptor-alpha (mRXR-alpha)/cds = (177,1580)/gb = X66223/ | 320.6 | A | 510 | P | 500.8 | P |
| | | gi = 54021/ug = Mm 3470/len = 2088/STRA = for | | | | | | |
| 20 | 92353_at | Cluster Incl Y18723 Mus musculus mRNA for secreted senne protease (BSSP)/cds = (237,977)/gb = Y18723/gi = 4220545/ | 45.1 | A | 1360.8 | P | 2076 | P |
| | | ug = Mm 3944/len = 1122/STRA = rev | | | | | | |
| 20 | 93866_s_at | Cluster Incl D00613 Matrix gamma-carboxyglutamate (gia) protein/cds = (18,332)/gb = D00613/gi = 220478/ug = Mm 87793/ | 90.5 | P | 131.2 | A | 295.3 | P |
| | | len = 540/STRA = for | | | | | | |
| 20 | 94120_s_at | Cluster Incl AJ005564 Small proline-nch protein 2F/cds = (23,253)/gb = AJ005564/gi = 3093366/ug = Mm 10692/len = 570/ | 123.5 | A | 2799.2 | P | 1214.3 | P |
| | | STRA = for | | | | | | |
| 20 | 98772_at | Cluster Incl U27267.Small inducible cytokine B subfamily, member 5/cds = (65,463)/gb = U27267/gi = 950158/ug = Mm 4660/ | 39.9 | A | 172.7 | P | 274.6 | P |
| | | len = 1519/STRA = for | | | | | | |
| 20 | 98833_at | Cluster Incl X66402 Matrix metalloproteinase 3/cds = (24,1457)/gb = X66402/gi = 296167/ug = Mm.4993/len = 1740/ | −8.1 | A | 415 | P | 603.6 | P |
| | | STRA = for | | | | | | |

TABLE 2
Genes clustering closely to actin

| Affymetrix | Accession | |
|---|---|---|
| Accession | number | Descriptions |
| 92573_at | AB021743 | Mus musculus mRNA for PR65 |
| | | (type 2A serine/threonine |
| | | protein phosphatase A subunit), complete cds |
| 92621_at | X97982 | M. musculus mRNA for poly(C)-binding |
| | | protein, splice variant E |
| 93346_at | M15668 | Mus musculus X chromosome-linked |
| | | phosphoglycerate kinase (pgk-1) mRNA |
| 95682_at | AB026432 | Mus musculus mRNA for XPE UV-damaged |
| | | DNA binding factor, complete cds |
| 96876_at | U34259 | Mus musculus Golgi 4-transmembrane |
| | | spanning transporter MTP mRNA, complete |
| | | cds |
| 99668_at | U86405 | Mus musculus brain amphiphysin 2 mRNA, |
| | | complete cds |
| 101028_i_at | M15501 | Actin, alpha, cardiac/cds = (0,1127)/ |
| | | gb = M15501 |
| 101030_at | X99963 | M. musculus rhoB gene/cds = (0,590)/ |
| | | gb = X99963 |
| 101554_at | U57524 | Mus musculus I kappa B alpha gene, |
| | | exons 2-6, partial cds |
| 104598_at | X61940 | Mouse mRNA for a growth factor-inducible |
| | | immediate early gene (3CH134) |

TABLE 3
Genes clustering closely to MRP-8

| Affymetrix | Accession | |
|---|---|---|
| Accession | number | Descriptions |
| 96092_at | M96827 | C57BL/6J ob/ob haptoglobin mRNA, |
| | | complete cds |
| 97529_at | AJ002390 | Annexin 8 |
| 103589_at | AF053235 | Mus musculus keratin 16 mRNA, complete |
| | | cds |
| 104370_s_at | K02108 | Keratin complex 2, gene 6A |
| 103448_at | M83218 | Mus musculus intracellular calcium-binding |
| | | protein (MRP8) mRNA |
| 94121_at | AJ005566 | Small proline-rich protein 2H |
| 99701_f_at | AJ005560 | Small proline-rich protein |
| 101024_i_at | AJ005559 | Small proline-rich protein 2A |

TABLE 4
Genes clustering closely to MMP-3

| Affymetrix | Accession | |
|---|---|---|
| Accession | number | Descriptions |
| 100127_at | M35523 | Cellular retinoic acid binding protein II |
| 94391_at | AW123650 | UI-M-BH2.1-api-g-08-0-UI.s1 Mus musculus |
| | | cDNA, 3 end |
| 97950_at | X75129 | Xanthine dehydrogenase |
| 102255_at | AB015978 | Oncostatin receptor |
| 103723_at | AA608387 | vo42b10.r1 Mus musculus cDNA, 5 end |
| 99159_at | AI842675 | UI-M-AO1-aeo-b-01-0-UI.s1 Mus musculus |
| | | cDNA, 3 end |
| 98018_at | L39017 | Protein C receptor, endothelial |
| 94224_s_at | M74123 | Mus musculus (strain C57B1/6) mRNA |
| | | sequence |
| 100882_at | AF003525 | Defensin beta 1 |
| 100884_at | U04204 | Fibroblast growth factor regulated protein |
| 101384_at | AI852933 | UI-M-BH0-aiz-c-09-0-UI.s1 Mus musculus |
| | | cDNA, 3 end |
| 103080_at | U15635 | Mus musculus IFN-gamma induced |
| | | (Mg11) mRNA |
| 103634_at | U51992 | Interferon dependent positive acting |
| | | transcription factor 3 gamma |
| 96123_at | X99347 | M. musculus mRNA for LPS-binding protein |
| 98772_at | U27267 | Small inducible cytokine B subfamily, |
| | | member 5 |
| 98833_at | X66402 | Matrix metalloproteinase 3 |

TABLE 5
Genes clustering closely to Cathepsin-S

| Affymetrix | Accession | |
|---|---|---|
| Accession | number | Descriptions |
| 93118_at | AI844131 | UI-M-AL1-ahj-d-09-0-UI.s1 Mus musculus cDNA, 3 end |
| 93274_at | M38381 | CDC-like kinase |
| 93806_at | AI848671 | UI-M-AH1-agv-e-02-0-UI.s1 Mus musculus cDNA, 3 end |
| 94081_at | AW124390 | UI-M-BH2.1-ape-c-03-0-UI.s1 Mus musculus cDNA, 3 end |
| 98946_at | AF033186 | Mus musculus WSB-1 mRNA, complete cds/cds = (0,1265)/ |
| 98087_at | AW048562 | UI-M-BH1-alx-e-05-0-UI.s1 Mus musculus cDNA, 3 end |
| 99577_at | M57647 | Mouse mast cell growth factor (MGF) mRNA, complete cds |
| 102781_at | U37351 | Mus musculus Paneth cell enhanced expression PCEE |
| | | mRNA, complete cds |
| 103667_at | AA866655 | ud06h05.r1 Mus musculus cDNA, 5 end |
| 104056_at | AI573367 | ud42d02.y1 Mus musculus cDNA, 5 end |
| 94975_at | AI845607 | UI-M-AQ1-adx-e-10-0-UI.s1 Mus musculus cDNA, 3 end/ |
| 102302_at | L16992 | Mus musculus branched-chain alpha-ketoacid |
| | | dehydrogenase E1 beta-subunit mRNA sequence |
| 103753_at | AI159572 | vz77c07.r1 Mus musculus cDNA, 5 end |
| 104640_f_at | AI464596 | mz66c12.y1 Mus musculus cDNA, 5 end |
| 92502_at | X95504 | M.musculus mRNA for zinc finger protein |
| 92513_at | AJ002636 | Mus musculus mRNA for nuclear protein SA2/ |
| 92542_at | AI849035 | UI-M-AH1-agw-a-06-0-UI.s1 Mus musculus cDNA, 3 end |
| 96616_at | AI891629 | ul57e11.x1 Mus musculus cDNA, 3 end |
| 104070_at | AW047728 | UI-M-BH1-alk-a-01-0-UI.s1 Mus musculus cDNA, 3 end |
| 104193_at | AW047583 | UI-M-BH1-akm-b-04-0-UI.s1 Mus musculus cDNA, 3 end |
| 93775_at | AI841894 | UI-M-AO0-acd-d-09-0-UI.s1 Mus musculus cDNA, 3 end/ |
| 97869_at | AI844043 | UI-M-AL1-ahi-d-11-0-UI.s1 Mus musculus cDNA, 3 end |
| 97541_f_at | X00246 | Mouse mRNA with a Set 1 repetitive element for a class I |
| | | major histocompatibility complex(MHC) antigen |
| 95028_r_at | C77009 | C77009 Mus musculus cDNA, 3 end |
| 97421_at | U42385 | Fibroblast growth factor inducible 16 |
| 101923_at | U34277 | Mus musculus PAF acetylhydrolase mRNA, complete cds |
| 103202_at | AW047476 | UI-M-BH1-all-g-04-0-UI.s1 Mus musculus cDNA, 3 end |
| 92226_at | AA866971 | vx91g10.r1 Mus musculus cDNA, 5 end |
| 98893_at | AW048644 | UI-M-BH1-amd-d-08-0-UI.s1 Mus musculus cDNA, 3 end |
| 103016_s_at | X68273 | CD68 antigen |
| 94934_at | U04828 | Angiotensin II receptor, type 2 |
| 100880_at | AA816121 | vp44a0l.r1 Mus musculus cDNA, 5 end |
| 94818_at | AW047223 | UI-M-BH1-amb-e-09-0-UI.s1 Mus musculus cDNA, 3 end |
| 98045_s_at | U18869 | Mus musculus mitogen-responsive 96 kDa phosphoprotein |
| | | p96 mRNA, alternatively spliced p67 mRNA, and |
| | | alternatively spliced p93 mRNA, complete cds |
| 98543_at | AJ223208 | Mus musculus mRNA for cathepsin S, partial |
| 98562_at | X58861 | Complement component 1, q subcomponent, alpha |
| | | polypeptide |
| 100611_at | M21050 | Mouse lysozyme M gene |
| 98465_f_at | M31419 | Interferon activated gene 204 |
| 100397_at | AF024637 | TYRO protein tyrosine kinase binding protein |
| 103226_at | Z11974 | Mannose receptor, C type 1 |
| 95806_f_at | M68899 | Mouse mast cell chymase 2 mRNA, complete cds |
| 102104_f_at | AI504305 | v104g11x1 Mus musculus cDNA, 3 end |
| 99507_at | M21247 | Uncoupling protein, mitochondrial |
| 92718_at | AI158810 | ud38d09.r1 Mus musculus cDNA, 5 end |
| 94774_at | M31418 | Interferon activated gene 202 |
| 101635_f_at | M16360 | Mouse major urinary protein V (MUP V) mRNA, complete |
| | | cds |
| 101682_f_at | M16358 | Major urinary protein 4 |

TABLE 6
Genes clustering closely to TGF-beta

| Affymetrix | Accession | |
|---|---|---|
| Accession | number | Descriptions |
| 92793_at | X57796 | Tumor necrosis factor receptor superfamily, |
| | | member 1a |
| 92877_at | L19932 | Transforming growth factor, beta induced, |
| | | 68 kDa/ |
| 99604_at | AI848713 | UI-M-AJ1-ahf-a-10-0-UI.s1 Mus musculus |
| | | cDNA, 3 end |
| 101040_at | D38117 | Mus musculus mRNA for m-calpain large |
| | | subunit, complete cds |
| 101982_at | X98475 | M. musculus VASP gene |
| 96187_at | AI837021 | UI-M-AP0-abj-a-11-0-UI.s2 Mus musculus |
| | | cDNA, 3 end |

Sequence Information

SEQ ID Nos 1 to 12 are DNA sequences corresponding to expressed sequence tags (ESTs)(mRNA) from Mus musculus found in clusters 2 and 7. The genes that encode these ESTs encode proteins important in the early wound healing response

SEQ ID Nos 13 to 29 are DNA sequences corresponding to expressed sequence tags (mRNA) from Mus musculus from clusters 5 and 6, the genes that encode these ESTs encode proteins that are involved in the inflammatory response and are not required for wound healing.

SEQ ID NOS 30 to 39 are DNA sequences corresponding to expressed sequence tags (mRNA) from Mus musculus found in cluster 20, the genes that encode these ESTs encode proteins important in the late, advanced phase of wound healing, that is genes involved in cessation of the healing response, maturation and remodelling of the repaired tissue.

In the list below, the sequence identification number is given, followed by the Accession number and then the Affymetrix reference number.

SEQ ID NO. 1

>AI846118 98910_at
tttttttttttttttttgaagttagaaaagtcttttaagtttttaaattaaaattgcaccctggtgcgatgactctaatttgggaactcaatccaa
aggcagactctcctgagtgctggtcctctggcctggacacacagagactgggagcctgtcggacagttagaacagggaagggg
cagaagcacccacatcagagggcagccaagtcttgacatctacctggtgacaagg

SEQ ID NO. 2

>AA655823 100466_f_at
ttgaaccgcgggcggcctgggctccttctgccatggcccccgcgctcaggtccttgctgtcgccacggactttactgctgctgctg
ctgagcctggcgctgctgggcgccgccgagcccgccaccgggagcgctgtccccgctcagagccgcccgtgcgtggattgcc
acgagtttgaattcatgcagcgcgcccttcaggacctacggaaaaccgcctacagcctggacgcacggacggagaccctcctgc
tgcaggctgagcgccgggctctgtgtgcctgctggccagctggacgctgaagatctttggccagtgatattccttgtcaataaatgc
ctgctagcatgccgnga

SEQ ID NO. 3

>AI846077 103433_at
tttttttttttttttggttttagcagtatgtttttatttagggaacacagtatttgcaatagttctgacagccgtaaggccctccattttactagc
tgtttgctttgcaataaaatagccagaaattctgatatcaacattaataggatatttcttcatgcaggttgtaggagtatatac
attgtataacactgaagaaattaatacatatgtacattatttacatagtaactatttattgtaaatagaaatgttttctaagataaaaagaaa
tcacaacatggtttctggtctgt

SEQ ID NO. 4

>AI642048 104149_at
gtctgtaaaaatctgtttaataaatatacatcttagaagtaccaaaataattaccaacaaaatacaacatatacaacatttacaagaag
gcgacacagaccttagttgggggcgacttttaagcacatgccactgaacacctggctcttacatgggaggacacactgggctcact
tactaggtctatggtggttcaatcaaaagcacaataaataaaacgtggtcctttcattaggttctggaaaatcacctccccccccccc
aaaaaaaatcccacaaacatgaaccttaagagacattttctttgaatttcagtgatctgtttccccggatttcacaaagacaacagccg
aatcaccccagtaaaatgcctgggtctaggcgctgtgtggtgtggtgctaagtataccctttctcattttttttctttttct

SEQ ID NO. 5

>AW124785 104410_at
tttttttttttttttttcaggtctcattttcgtttatttgaaattcggtgctcgtgtaagttttttctcttcccctcaaattttatttcagtaaaagga
gacttgggcgaggtggataccccacagccggattcttccccccctgccccccagggtggctaatgctatctggggaagtcgtcat
agggaagagaactatgggtgggctcctgcctgaggcctccaatctcagcccagtggacatatcacaggcagcttaaaaaaaaaa
ccctaaaaaaaacaccccaaaacacacatttaaataggtattcaagacagctttaaaaaatgcacccactcacacccccctccctttt
ctttttggaaaaaaaaataggaaaaaaaaaaaaaccaaaccgaattctcgcttggcctcta

SEQ ID NO. 6

>AW047643 104477_at
tttttttttttttttttaaattcaagtgcctccatcttgtttacctatcactttaaaaagtactttaagtataagcttcaagagttttgagttctag
agttttgcctggctgttgagatttggtgcaaattctcatgtgtgtttatataggaaggtgagctgaccatccactgcagaggatggagg
taattaacatgatctgtgctctttacaaaagagaatccagtgacctggcctgtgtgtgtgtgtgcgcgcgcgtgcacacacgctcgct
agcgctcacattatgcatgaaaacttgattttcctgaagcatccatttccaaattggatagggatgaaaaagtaaaatttccattggatt
caattagaagc

SEQ ID NO. 7

>AA764261 104533_at
tttttttaagacaaaaggtttttattcaaaaaatgccaagtataaaaaaaaaaaataagatctcttttattcccctgtacagtatactttact
cagataaaaccagcaggctgcacgctgctcaaagcacagccccagagaggatccggaagcacaccacaccggaactgtcttat
gtacagtcagcagaccctagacccactccagcaagcagccaagggggcccctggagaagggggctacagcagctgtcccacc
ggagccccaacaccccagcaagcgtcacagccctcccctaaaaggtccccaagtcaggagga

SEQ ID NO. 8

>AI845237 94255_g_at
tttttttttttttttctgacacagtagtaatccttccagactccttacacaatattacaactcccccagtacataaatgttcctataccatgca
cacagcaacatggggtccactgatgtcgcaggcgacttttctaatggtggaacatagcacctcaagttctgccatctacacagtgaa
gggacgtgatggtcggggctccagagtgacagcaaactgcctcttgggtgacacgcttgtggcgaagtgcctcc

SEQ ID NO. 9

>AI845579 94555_at
tttttttttttttttttcatccacaaaaaaaaaccaattttattaatccaagaaatgaaaaccatgtccctatggcttctagccccctcaaaa
ggatgaggctgggttcatgaacctggggtagaatgtcccctatccctcaccctcagcttattagactggaaaaagagtttgtgcaaa
taggacggtcaccagaggtaccaagtatgggggtgtcaggccagggcagcagatgaaggaggtgagggatctgt

SEQ ID NO. 10

>AB023957 96132_at
ctgacaaggagatctgtggagtttttctccaagtgaaccaatcccctgtgtcctggctcacactgtggttagggtgggcacatccact
ctgccatctttaacacacagaaaccaaacatcagtgctccagtagcttactaatggaaagaggttggggtgagactcacccacaga
tgagccactctgctaggagccagctgtccctgtgctccttggtggggtctaggaggagtcaccctgttactagaattaccagcttgat
tcggcttcctctctgcatttccctggttctgccccacctgcgaggtccagagccatctcattcagggttcgccgcttcacagtcatccg
aagggaggaggggatggccgccacccattcctttgtattattgcaactggtgctataaacaccaagtgctattgctcacagagacc
catgaaggtaggtggaggagagacaaaaaacctagctggaactgaaagcagacatggagaccaggacccaggagaagagtc
agtcgtgtatttaagagttcactttgtgaagcagtagctttcagtgcggtgttaaattataacgaaggcatggagtaccagggtgcttt
atatttagattatattttaagtcatggtgcatacttgagggggtttattaaaaatgaaagtactaacttaatgtctactaagtttaataaaca
aagaatttggaagtaaaaaaaaaaagct

SEQ ID NO. 11

>AW125478 96920_at
tttttttttttttttatgaagaagtattttaatagctgaaactcaaagtcactgtactccgggttccaggattcctaagaggcatttttggcca
tctcagaagccagggtcacccacctgtggtctcggggcatcaaattcctctgagtgccgactcggaggaaaagtgtaaacacgcc
caagaccatggctgcgaggaccttcctcttgcctacgcatccgtcaactgcattagtcagacaacttaggctaagggaagggaca
aatgttgactgtgtaagcaggaacagcccaagcttgtaaggaaaagcacgctgcatacatagcatacagaaggcaggacttctgc
ctgtgcacctgcaacattacatttgttttgcccgcaaactatcaagaaggattctgtcgggaggggtgttccctgaacaagcaaaatg
ttggacaaaggtcccgggtgtcctgcgtcac

SEQ ID NO. 12

>AW049031 98083_at
tttttttttttttttttgctgataagaattctttttatgttattcgaataaaaaatacattcatacagaaatataacaatctcgcaaaaaacaatt
tcaaataaaatcttgtaaaacaaaattttacaaaaatcttacaaagattctttagataacagggtgcttcaaaaaaaaagaaataaaga
aatttcactaatagaaatttttttttttaatttcaagcaaaagttcctgcttgattgaggctcagttgtcacctgaccagaatggactgctta
gtattaaagttacagcatcgacacggacggcacccagccccagccagtccagcaacgtcgctgtgtttcataagtgagacgcgcc
agcacaagtttcctctctcttctgtttaccttcttacttaatggaattgctatggataagcacacagcagggccaaaaaaggagttttcc
aaaatccagcaaatcaagtg

SEQ ID NO. 13

>AA816121 100880_at
cataaaacagctgaatacatacaaaactgtggttattgatgagctgttgcttttgttgcctttcttgctgtttattaactgacaaagaccat
gctaacaatcatctttatgaatgggtgctagctgatgggcagatcatttgtatgggctagataaaaatgttaataatgaagccatgtgc
tcaggtttgaattgaaaacaattaaaaaaaatcacttgctattttgttttcctttgaagaatataaagttatttcagttagatcatcagtaaa
gttttatctgggctaagaaaatttcccttgagttcttgtgct

SEQ ID NO. 14

>AI255271 102096_f_at
ttttttttttttttttttttttttttttttttttttttttttttaagggcaggggtaatctttttttgacccggaagaggaaaaaattcatgaaaaattgc
cgtgggtttttcaaaaaaaaatttaaagaactctttgtttcttgatgaggtatcctaggtaccatggggatagactggaattggccttctc
agggagacaggatggaatgcaaaccccaaaatttaaactggggatgctgtttggataggaagggatgatgggggacccctggg
gagaagcctccactcaacactggaggctcaggccattcttcattctcggccctggaggcaacgatgggcattggataggccaatg
atattttctttaaggattccatgctcctcacatagttgggcaaacctttccttgatgtctgacctcaaatctggttctcggccatagagcc
ccatcagcttgaaggttcccccatcctttccgtaatgagatgagccataagaaagttatcatagtctgcctaagtataggaaatggatt
gaatcatcatcccgcacagatattcaccagcttttttgtttgcagcaccctagataat

SEQ ID NO. 15

>AI504305 102104_f_at
tttttgtctttcccatgcttttattggttgatagagaacaaaagaatatcaaacaaatgtgtttacatatcacaggggacttgatctgtgca
taaaaacaaaataacaacaaaagcccttacatatagaacatacatncaaaaaagatttnttttngaaccaacagcatgaanacttctt
gagattcattgcatgccactagtttctatgcactagtgaataaaactgatatgataaaagacccacatcatagcagagattctgaatac
aaagactgctaatggttaactgttgctgttaattgcagggggcaagaaaatgtccctttgctccactacaatggcctgaaacattgnt
nggntttctttctttctttctttttttattacgtattttcttcaattacatttccaatgcgtatcccccaaagcccatacctccccccccactct
cctacccacccattcccattttttggcccctgcgttcccctgactggggcatataaaagttgcctgaccaatggccttctcttccagtg
atggccgactaggtcatcttttgatacatatgccgctag

SEQ ID NO. 16

>AA866655 103667_at
ggtaactacagcttagaaacccaatatcagttgtacttaacatctttaagagcatgatcataaaggctattttgacagttcattttaacat
ttagagttgataagggtttatatctcatctgtccatattgtttgcaaaggaacaaggttttatgtaggtgagagaaagcatttgtacga
agttagatttgaacatagataagataggttattccagttgagaatattgttcgaagaatgtctgtgaatgcagacatgggtcctgcttag
tgccatgggtagactgagtcttctgtaagtcatcataacatagtaattttggtcctgaattttacattaaattatttgaatatatacaga
cctaaattttaaaatctgtacatatataattttgatgtattaagatggataatattgctgatttaaatttaattatgcacatacttaaaaggac
cgaaatgtctggtaaagtaaattgtaaataaggatatgtagcctttaaccttttaaataattagattttttaatgtgggtctcctcag

SEQ ID NO 17

>AI159572 103753_at
gcttaacgtgtttgatacctaacttcatgatcacgatgacctgtccttaccaagagcaaagcaagacatagctgtggctttgcttctgt
aaccagctcatggggtgtgaatgtcatgaagttgtctcgctgatatatgtttaatgtcatgttatatgagatgatcatgtgatgtgtacag
actatgttgagaagtgccagtggtagtaactgtaaagtctgactcacaccattaatccctctaaagtacacagccttctgcctctgtgt
ggagttgtcagtacagctcgtcaaagaaaactgcctaatataaaaatcatatatatggtgataattttccctcttttgtagtctgcacaa
gatccataaaagattgtatttttattactatttaacgagtgattaaatgtagtctgcacagtgagcaagagttcacaattctttttatactgc
tggattttgttgtgcatcatttaa

SEQ ID NO. 18

>AI573367 104056_at
ggaacttcaatagaatatcgtcccttgtctgaggaaagaccacctcaaccacatgcctatctagagattgaataaaactgtgaggag
acactgtgtggatgccttaagatggctggcgctggctgcagggaacaatctatttcagctcatcttcataaagcccaagatgctccc
gtgcataaccttgttgaagaaatgagaaatgcggtgctggctactttgatgtgtctggggaggctggcgtcattggttaccatcatta
agtcactgtccagtgcccaagccttccttcactaccactcatatgtctacatccattcattcatctatgcaagcttgtgtttttgtggtttgt
attttacacctggacatcaggtttaacattggtgttttaaataatttttaaaccaaagtattttatgagtaagtctatgtgctctgtttttgt

SEQ ID NO. 19

>AI153421 96215_f_at
cttcggcacaggcgcacggtnaggagtttgcctctgcctcccgagtgctgggatgaaaggtgtgcaccaccacgcccgactcca
atttttttctttattattttttttgtgggtgtgaattgaaagtctaatacgtgtttctctgtttatggacccggatcccacgccagcgtccact
ggtcactgccgctgcccacagcgacgtcgcccacgagagcacacacgtagacccagacgccgtggtcaagaatctctgccatc
cccacgggacggacggacggacggactccacaaggtgcgcgtgtcaccgaggccgccagaatggagccactctcacgattct
caacagggcctgaccgcgtacagaaatgtccccctcaataaa

SEQ ID NO. 20

>AW047728 104070_at
tttttttttttttttttcacattctacttctgctttattgcacaatatctataagaaaatattaattttaaattagaaaacataaaaatgctttcaat
atagaagagaccatctgattttaaagacaatactctttaaggatgtctacccaggaaataacaaactttcacaggataccaaactgga
cagacgcttcagcgaaatcggtacaaagctgttttaaaaccagtctgagacacttaatgcagcctctattatcactcttcattaatagtc
tagataccactcaaagtgttccttacatttcccaatgttgaggcatcttcagtgtgatcttcagtgaacactatgaacatacagcatgcc
ttccatgtcagaagcagcactcggga

SEQ ID NO. 21

>AW047583 104193_at
tttttttttttttttttcaacacaaagctttcggcatattttattgtgaaatattggcttagaaaaaagtacaaaatgccaacagtcagatgta
tacaggtacagtattttgttaatctatacatgtgaccagcttaacaggctcatcggaaaatgcatatttagaccaatatactctaaaaat
gactgtaatattcacaggctattaatacatgataaactggagtctatatgaccctcactgggatatgctaactgcagcaaacaggcta
aaagcacaatacagcacaccatagtacctctggacaggtccacgtttaaagagtatcaattcattagatcaatacactacagatctg
ggactgagactcaatcactaatttatacaattgcaaaaaaaatttaggttataaaataaagttatgg

SEQ ID NO. 22

>AI464596 104640_f_at
ggcatcgttggtttcttggctttgtgcatgttaaacctggtatttctaatgatacagtttttcatatgtacataaatttttgtatgtaggtgtgt
cggggagggcagaacaacttgcaagtttgatctcaatgtgggggtcttaaactacctggtcttaaagctttctcttcttcctagg
gtgacacgagtgaaccctaccctaaaatggtttaaagataacatgaagggtccttaaaataaattgaagaaatCctCaaatCttCtgg
tgacctggatggacagcaaaggaatcaaatccctcaaataatttatttgacttaaattaaaaataaataaa

SEQ ID NO. 23

>AI848671 93806_at
tttttttttttttttttcacttaatcagttttattgaaattcatcagatgaaaagaatcagtgttccatagaaaaggattaatgttgtactttgtaa
ttttaaacaccaacaactgcttaacaaaaattcatgtgagttttacattttgttgttctttttacttaaggagtgaagtctgatttcaccaaa
actccctgtactcagtctttcccagtaaaagtaatctttgtttcaaaattccctattcacatatacttagggtcccaatgaagtgtccacat
ttagcagtacaattaacaatttacagtgtttttgaaagtggaactctttctaaagaacaaaatttctaaagggcaaatcagctcttaaatg
tgaaatttacaa

SEQ ID NO. 24

>AW124390 94081_at
tttttttttttttttttgacttttaaaggatttttatactatattaaaaaaaacacaaaataaaaaagggatccatcaacatatattttagaagtc
catccaagagtttcagtgtccaccagccatggacacaacagccatgcctgtgtcattgttcagtctgtagctcatgtaaggatcaag
gaggtgactttaagttacaatcaaacttgctctg

SEQ ID NO. 25

>AI845607 94975_at
tttttttttttttttgtgataatacaattttctttagtaaaaataatttacagcatcaataacatatacacaatttcataactgaactttacctcca
atatatttctatacaatacttaacattattgaacttaaaactgttatgctattttgttggctttaaataacaatgatttgtagttacttaagaga
tatataaggttttacagtcacatattatgcataaaattgataaaacaaacaaatcagcacagtcaccttgaaagggaaaataacccac
tggaaaaaagccaaagttcatttccttccccgttttttactccccaaatcaaatacagtatcattgtccagtaccttagcatcttctctc

SEQ ID NO. 26

>C77009 95028_r_at
gggaggaaanggtttatttggcttacactttcatactgcttactgttcatcaccaaggaagtngggactggaactcaagcaggtcaa
gaagcagaactgatgcagaggccacagaaggatgtttctttattagcttgcttcacctggcttgctcagactgctgtcttagagaacc
caagactaccagtgcagagagatggtcccacccacaaggggcctttccccttgatcactaattgagaaaatgtcttacatctggatc
tcatggaggcatttcccaactgaagctcctttctctgtgataacttcagcttgtgtcaagttgacacaaaattagccagtataattgaca
ccttgtcaatttgaggcgnnnncacatcactagtaatgctcaactcttagtttcttattcatgcccaagatatacacaactttgaaagcc
ccacagactttacatattaaaagttcaatgcttttaaaatatccaatatncgtg

SEQ ID NO. 27

>AI891629 96616_at
ttcacatagcatcaagtgactacagatacaacactagaattttgatgtgtattatttgaacacgtttatatatgagaaaaatttgaaattta
agttacaaagtgagtattttttagcacatttagtagtgactatttatataaactatcattttgtttgggtgaccaaatgaaatttgaacataa
ttaaagaacatataactttcaaacaacattaatacagttacagacctgttacactgagaattatgtacacgtattaaagaggactgagg
tttcttaaacccattcttttgaaaggtttttaatccttcttaatgtaattcagtgtaaaacttcaaaaagtctactcggtgtcacagcaggat
gaggtggaataatccctgctgatcactgagcaaactgtgtttgtcatctgattctcctgacaaccactatacttga

SEQ ID NO. 28

>AA684508 97838_at
gttcatctcaaagccctttacactttggagccaggcctgttcaattttacaaataacacgttttgcttgtagtcagggtgctgtgacaac
atgaagagttcgagctacctcccaggatgagtgcaggttttcctggtgacaaatctcaggcattcaaaggttctgttagcagtgccat
acttgtgggtttacagagatggtgtgtctggctgagaacaaaggagccgtaccagggaagttgaagcctatcttcattttcaagtgc
aggctaaaataaggtctgaggaaaagaatgagcagcgtgtgaaggataggaacagaaatcatggaagatctgtttgtccaggcca
gtgtttcaccataagagatcactttaaaataaatgagatgaaataatt

SEQ ID NO. 29

>AW048644 98893_at
tttttttttttttttttcactttgcaaactgttgtttttatttggattaaaaagctttgatgaacaaacatatgatacatacatggtatagaacca
gttaccacttaaaacctcagatgatatagtacagaaattagaataaaccaaattttaatcaggtctggaaacaaacgttcaagctgaa
agctgtaacatccatttggagattttacacacctgtcccttacccactcagttcttccttagaaggggaaggcagacttagtaaaacca
atgttttttaagtcagctgactgacattctattttaaacctataggtccccaacccaatataaacaaaattcttcacaggaacttacatttt
gaaaacaattaggccattatatttcctaagccacattactacagttcttaaaagtcatgagaaag

SEQ ID NO. 30

>AI844810 103416_at
tttttttttttttttttattcatgcttgcctagggatggggaatagatcattcaataaaaacatacagtaaaaacagggggtggggagggg
gaaggcttatcatgtacaatgtttaaactacaatagtgatgtaccttaattacttccatgcacacaagtctaacattacaagtttttaaaa
aataaacaccattaagacttctaggagcattttataataaattcctaattttttctttgtagatagatcaagcacctccaaaatacagattc
ctatacacagtgagcactttacttaacgtacatggacagcctcaggacgagctgacgtctcggatcagctcggcaggcaacaaac
catagtgccaaatggaaagaagggcagttgcaaataaacttaaaactaagttaacttttataattaaatacagaaaatatactgatttg
ctaaaaataaataagatgtgatgtatttaacacttcactataaagaatgaacaccatgacatcctcgtgccg

SEQ ID NO. 31

>AA608387 103723_at
ttcgtcgacatgcaatagcagggggaaaggggacaggtaacaggagtacctttgttttaacagacaaaagtaatctccaggcctta
gggactcataaagatatgagtacattgcttattggaaacttgtaggttcacatattattttgactaaggtatatggatttccccccactta
cataagtaaacccaacataaggaggaccattccttttctttcattcagatgacctttttctgaatctgggcactgaagggatgcataaa
ataatgttaatgttttcagtaatgtcttcaagtagaagactttctgttgaactacttataagaaagtaaaataaatattgtctttttgtatgtc
aaaaaaaaaaaaaaaaaaaaaaaaaaaagg

SEQ ID NO. 32

>AA689670 104737_at
aattcgtcgacatggcacagcttcctccctaggcgtgagactccggctccttcactatgagacttctagccctttccggtctgctctgc
atgctgctcctctgtttctgcattttctcctcagaagggagaagacatcctgccaagtccttgaaactcaggcgctgctgtcacctatc
tcctagatccaagctgacaacctggaaaggaaaccacacaaggccctgcagactctgcag

SEQ ID NO. 33

>AW060684 94433_at
tttttttttttttttttgattaatcatgtatttattttagttcagttaaaacaaacatacattgtttcattgaaaccggtgtagcatttactctctgc
caacaggccacagaggcatgttggcctctaacagtacagctgggacatttacactatatacatacaaggagtcctctttcattgagat
cacacttgacatgttaccaaatagccatgtggtagctactgcattttgaaaatttagacctcatttaaagctttagattatctgaacttaa
gttgatgtccaaccagtttattgcaaatacaatttaaacaagttattttcaattttagtattatacacaatgtcaatttcttaagattgaattct
ctgtacaagta

SEQ ID NO. 34

>AW125178 94486_at
tttttttttttttttttgaccacacagacatggagttaccaattgtagaaatcgaaaaaattggcatcaactctcgaagtcagagtgaactc
ttgcctgcgggttggcttgactacgcccaggcactgagctgcctcaaccagctagggagctatgatgatgctgagtcctgttttcat
gatgtcaccagatgtatgtagtaggtaatttgtctcacaaaggcctttgtaccaatggctgtgaagcttg

SEQ ID NO. 35

>AI835858 95542_at
tttttttttttttttttaattacgcaaaaatttatttgaccaaaatgtagaaaaagtgatactattacgtatgatacaatagcaagaacataaa
gaacagagtagattttatcaattgcagtttgctcaaagttcatctccctggtagtgtgatgcttaataaataggagtgaggggcag
ggttcagataagctagaagcagggtgactttcagtcaggactgtaaacttgagttggccccgcactgctggggaatgtggaatgttt
cagtgtgagatgttaactgagaaaggaaaactacaaagccaaaaaatgtgcagcctacagagcactccacatccagttcaatcag
gaggtcttggtcttttattagtgtcattcccgaaggaattcaagtcttggcaaagtcaagcttccatttgttgttccctgaacaatggaga
tgaagttaagccaatttctttttcta

SEQ ID NO. 36

>AW121246 96281_at
tttttttttcctactagactaattaaaaaaaaaagtcaacattgatatcgattacttgcatgaagacacaggaaaacaggcgacataca
ctccttaaagacacacaagataagaagatgaaacttcaggtacataagaagtctgtacaaaaagctagatttgatactctcaaaaag
tgaagggtcctacaacagcatagagaaataatttaatgccttccagaacagaactccagctctgtggaggttttcctattctatgggg
gcagatctcatgccaacccacagagcaggcgcttccacctcctatccgtttatgcggtagttttcatggatttccggccggcctcgtg
ccg

SEQ ID NO. 37

>AW214159 96896_at
atgttgaaaaatcttatttattttcttaattaaaaagaagtatatttgtgttaaatttagaatatttagctttccattgatcttcccaatacttgtc
agatagatactaatagtgctcagaactgtgaaagttaatatccaataccatattttctaagaagaccctcaaggcattgggga
ttttaatttaaaaataaaagaattttaattagcattg

SEQ ID NO. 38

>AW123953 97904_at
tttttttttttttttttgaaaaagaaattttttgcctttattagaatggcattaggccttaaatatgccaattttggtaatcacattattgttttaat
aagaaacgactctacagaattgcaatactggtccaacagtcttgtccttcttttaaagcaagaaacagaatgtaagtaaccagaaag
cagggcaggcatcggctaacccaggagactagcttcttagatccaagcatttgcagagagaaccgttgggctggggaggggtgg
agcagctcgagataactggaacccagagtgtgcgccaagtcccatgaggctgcttgttgaactcatcttttccttggtcacattgtttc
cctc

SEQ ID NO. 39

>AI842675 99159_at
tttttttttttttttttcaagagaaaataggatttatttatcacatttgagatgaacacacaggactgtcccagaaatcttttacatatatattat
tgatagattggatttattcagcatacagaaaaagcaaagcctgccataagacacttccctcttgagaacagcaagatgtcaggcagt
gtctgtaagttttcaccacgggaattcacattgagcaaaaagaccgggctcacaggtccgttcccaggtcctgctgcttctatttcaa
aggctttca

[image]

[image]

[image]

[image]

[image]

[image]

[image]

[image]

1. A gene comprising a sequence according to any one of SEQ ID Nos. 1 to 39.

2. A gene comprising a sequence according to SEQ ID No. 32.

3. A gene comprising a sequence according to SEQ ID No. 19.

4. An isolated and purified protein encoded by a gene according to any one of claims 1 to 3.

5. A human homologue of a gene according to any one of claims 1 to 3 or of a protein according to claim 4.

6. A biomarker derived from a gene according to any one of claims 1 to 3 or claim 5.

7. A method for identification of a compound useful in the treatment of wounds or of conditions characterised by an excessive or impaired wound healing response, which comprises contacting a compound with a protein encoded by a gene comprising a sequence identified by one of the accession numbers of cluster 2, 7, 11, 16, or 20 or a mammalian homologue thereof and detecting specific binding of the chemical compound to the protein.

8. A method for identification of a compound useful for the treatment or reduction of inflammation which comprises contacting a compound with a protein encoded by a gene comprising a sequence identified by one of the accession numbers of cluster 5 or 6 or a mammalian homologue thereof and detecting specific binding of the chemical compound to the protein.

9. An antagonist or inhibitor of a protein encoded by a gene comprising a sequence identified by one of the accession numbers of cluster 2, 7, 11, 16, or 20 or a mammalian homologue thereof, for use in the treatment of a disease or disorder characterised by excessive wound healing response, such as scarring, fibrosis, restenosis post angioplasty, psoriasis, post-traumatic/surgical adhesions of the peritoneal cavity, joints and ligaments, benign prostatic hyperplasia, glaucoma, or peripheral nerve injury.

10. The use of an antagonist or inhibitor of a protein encoded by a gene comprising a sequence identified by one of the accession numbers of cluster 2, 7, 11, 16, or 20 or a mammalian homologue thereof, in the manufacture of a medicament for the treatment of a disease or disorder characterised by excessive wound healing response, such as scarring, fibrosis, restenosis post angioplasty, psoriasis, post-traumatic/surgical adhesions of the peritoneal cavity, joints and ligaments, benign prostatic hyperplasia, glaucoma, or peripheral nerve injury.

11. A method for treatment of a disease or disorder characterised by excessive wound healing response, such as scarring, fibrosis, restenosis post angioplasty, psoriasis, post-traumatic/surgical adhesions of the peritoneal cavity, joints and ligaments, benign prostatic hyperplasia, glaucoma, or peripheral nerve injury, comprising administering to a mammal in need of such treatment a therapeutically effective amount of an antagonist or inhibitor of a protein encoded by a gene comprising a sequence identified by one of the accession numbers of cluster 2, 7, 11, 16, or 20 or a mammalian homologue thereof.

12. An agonist or activator of a protein encoded by a gene comprising a sequence identified by one of the accession numbers of cluster 2, 7, 11, 16, or 20 or a mammalian homologue thereof, for use in the treatment of a disease or disorder characterised by an impaired healing response, such as chronic dermal ulcers, oral mucocystis, emphysema, ulcerative diseases of the gastro-intestinal (GI) tract, or cystitis.

13. The use of an agonist or activator of a protein encoded by a gene comprising a sequence identified by one of the accession numbers of cluster 2, 7, 11, 16, or 20 or a mammalian homologue thereof, in the manufacture of a medicament for the treatment of a disease or disorder characterised by an impaired healing response, such as chronic dermal ulcers, oral mucocystis, emphysema, ulcerative diseases of the gastro-intestinal (GI) tract, or cystitis.

14. A method for treatment of a disease or disorder characterised by an impaired healing response, such as chronic dermal ulcers, oral mucocystis, emphysema, ulcerative diseases of the gastrointestinal (GI) tract, or cystitis, comprising administering to a mammal in need of such treatment a therapeutically effective amount of an agonist or activator of a protein encoded by a gene comprising a sequence identified by one of the accession numbers of cluster 2, 7, 11, 16, or 20 or a mammalian homologue thereof.

15. An antagonist or inhibitor of a protein encoded by a gene comprising a sequence identified by one of the accession numbers of cluster 5 or 6 or a mammalian homologue thereof for the treatment or prevention of inflammation.

16. The use of an antagonist or inhibitor of a protein encoded by a gene comprising a sequence identified by one of the accession numbers of cluster 5 or 6 or a mammalian homologue thereof, in the manufacture of a medicament for the treatment or prevention of inflammation.

17. A method for treatment or prevention of inflammation comprising administering to a mammal in need of such treatment a therapeutically effective amount of an antagonist or inhibitor of a protein encoded by a gene comprising a sequence identified by one of the accession numbers of cluster 5 or 6 or a mammalian homologue thereof for the treatment or prevention of inflammation.

18. A method for investigating wound repair in a mammal comprising: taking a wound tissue sample at one or more time points, isolating mRNA from the sample (or samples), determining for the, or each time point, the level of mRNA expression from one or more of the genes including the sequence of one or more of the accession numbers grouped in clusters 2, 7, 11, 16 and 20 or a homologue thereof, comparing the level of mRNA expression of the, or each, gene from the wound tissue sample at a given time point with the level of mRNA expression of that gene in a control sample of mRNA taken from non wounded tissue and determining whether the level of mRNA expression of the, or each, gene differs in the wound tissue sample relative to a control sample taken from unwounded skin.

19. A method for identification of a mammal with an impaired wound healing response comprising: taking a tissue sample from a test subject, isolating mRNA from the sample, determining the level of mRNA expression from one or more of the genes including the sequence of one or more of the accession numbers grouped in clusters 2, 7, 11, 16 and 20 and comparing the mRNA expression level detected in the test subject with the mRNA expression level of the or each gene a control subject with a normal wound healing response; wherein a difference in mRNA expression levels of one or more of said genes indicates that the test subject has an impaired wound healing response.
